SEQUENCE LISTING (9 sequences)

SEQ ID NO: 1 - protein, 5 aa, Homo sapiens
GGGGS

SEQ ID NO: 2 - protein, 10 aa, Homo sapiens
GGGGSGGGGS

SEQ ID NO: 3 - protein, 15 aa, Homo sapiens
GGGGSGGGGSGGGGS

SEQ ID NO: 4 - protein, 20 aa, Homo sapiens
GGGGSGGGGSGGGGSGGGGS

SEQ ID NO: 5 - protein, 25 aa, Homo sapiens
GGGGSGGGGSGGGGSGGGGSGGGGS

SEQ ID NO: 6 - protein, 330 aa, Homo sapiens [sequence omitted]

SEQ ID NO: 7 - protein, 107 aa, Homo sapiens [sequence omitted]

SEQ ID NO: 8 - protein, 6 aa, Homo sapiens
HHHHHH

SEQ ID NO: 9 - protein, 5 aa, Homo sapiens
LPXTG

(19) United States
(12) Patent Application Publication
(10) Pub. No.: US 2026/0034238 A1
(43) Pub. Date: Feb. 5, 2026

(54) LINKERS, DRUG LINKERS AND CONJUGATES THEREOF AND METHODS OF USING THE SAME

(71) Applicant: GENMAB A/S, Valby (DK)

(72) Inventors: Haidong LIU, Suzhou, Jiangsu (CN); Guobao WANG, Suzhou, Jiangsu (CN); Xiaolong JIANG, Suzhou, Jiangsu (CN); Xiao SHANG, Woodinville, WA (US)

(21) Appl. No.: 19/146,799

(22) PCT Filed: Jan. 11, 2024
(86) PCT No.: PCT/CN2024/071901

§ 371 Date: Jul. 9, 2025

(30) Foreign Application Priority Data
Jan. 11, 2023 (WO) PCT/CN2023/071778

(51) Int. Cl. A61K 47/68; A61P 35/00

(52) U.S. Cl. CPC A61K 47/6889; A61K 47/68031; A61K 47/68037; A61P 35/00

(57) ABSTRACT

It provides linker compounds, or stereoisomers or salts thereof, including (a) a linker unit having from 1 to 4 attachment sites for a drug unit and having one of the following structures (i) or (ii): (b) at least one polar group containing a polymer unit, optionally a sugar unit, optionally a carboxyl unit, and combinations thereof; and (c) optionally a stretcher group having an attachment site for a targeting group, wherein a—is an attachment site to an enzyme-cleavable group; (3—is an attachment site to the at least one polar group; 6—is H, an attachment site to at least one of the drug unit, or an attachment site to a linking group attached to the at least one of the drug unit. It also provides drug-linker compounds and conjugates formed from linker compounds, as well as related pharmaceutical compositions and methods.

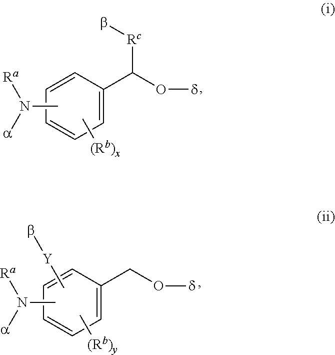

### CROSS REFERENCE TO RELATED APPLICATIONS

This application claims priority to International Application No. PCT/CN2023/071778 filed on Jan. 11, 2023, the entire contents of which are hereby incorporated by reference.

### BACKGROUND

A great deal of interest has surrounded the use of monoclonal antibodies (mAbs) for the targeted delivery of cytotoxic agents to cells associated with disease, such as cancer cells and other cells, in the form of antibody drug conjugates (or ADCs). The design of antibody drug conjugates, by attaching a cytotoxic agent, immune modulatory agent or other agent (collectively a “drug”) to an antibody, typically via a linker, involves consideration of a variety of factors. These factors include the identity and location of the chemical group for attachment of the drug, the mechanism of drug release, the structural element(s) (if any) providing release of the drug, and structural modification of the released free drug, if any. If the drug is released in the extracellular environment, the released form of the drug must able to reach its target. If the drug is to be released after antibody internalization, the structural elements and mechanism of drug release must be consonant with the intracellular trafficking of the conjugate.

Another important factor in the design of antibody drug conjugates is the amount of drug that can be delivered per targeting group (i.e., the number of drugs attached to each targeting group (e.g., an antibody), referred to as the drug load or drug loading). Historically, assumptions were that higher drugs loads were superior to lower drug loads (e.g., 8-loads vs 4-loads). The rationale was that higher loaded conjugates would deliver more drug (e.g., cytotoxic agent) to the target cells. This rationale was supported by the observations that conjugates with higher drug loadings were more active against cell lines in vitro. Certain later studies revealed, however, that this assumption was not confirmed in animal models. Conjugates having drug loads of 4 or 8 of certain auristatins were observed to have similar activities in mouse models. See, e.g., Hamblett et al., Clinical Cancer Res. 10:7063-70 (2004). Hamblett et al. further reported that the higher loaded ADCs were cleared more quickly from circulation in animal models. This faster clearance suggested a PK liability for higher loaded species as compared to lower loaded species. See Hamblett et al. In addition, higher loaded conjugates had lower maximum tolerated doses (MTDs) in mice, and as a result had narrower reported therapeutic indices. Id. In contrast, ADCs with a drug loading of 2 at engineered sites in a monoclonal antibody were reported to have the same or better PK and therapeutic indices as compared to certain 4-loaded ADCs. For example, see Junutula et al., Clinical Cancer Res. 16:4769 (2010). Thus, recent trends are to develop ADCs with low drug loadings.

There is a need, therefore, for antibody drug conjugate formats (and more generally for formats for other conjugates), that allow for higher drug loading, but that maintain other characteristics of lower loaded conjugates, such as favorable PK properties. Surprisingly, the present invention addresses those needs.

### SUMMARY

Provided herein are Linkers having hydrophilic characteristics that maintain the intrinsic properties of antibodies conjugated with the Linkers and drugs. In particular, the Linkers aid in maintaining the hydrophilic properties of the antibodies when conjugated at higher drug loading and/or to hydrophobic drugs and other agents. Also provided are Drug-Linkers and conjugates comprising the Linkers, as well as methods of using such conjugates for the treatment of cancer and other diseases.

In some embodiments, provided are Linker compounds, or a stereoisomers or salts thereof, comprising:
(a) a Linker unit having from 1 to 4 attachment sites for a Drug unit and having one of the following structures (i) or (ii):

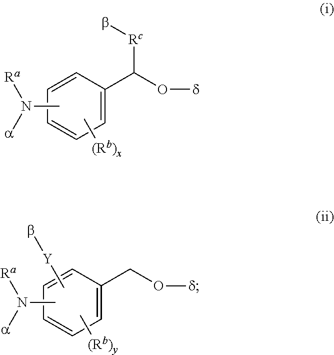

(b) at least one Polar group comprising a Polymer unit, optionally a Sugar unit, optionally a Carboxyl unit, and combinations thereof; and
(c) optionally a Stretcher group having an attachment site for a Targeting group;
   wherein:
α— is an attachment site to an enzyme-cleavable group;
β— is an attachment site to the at least one Polar group;
δ— is H, an attachment site to at least one of the Drug units, or an attachment site to a linking group attached to the at least one of the Drug units;
the Polymer unit comprises a polyamide, a polyether, or a combination thereof, wherein the polyether comprises a hydroxyl group, a polyhydroxyl group, a sugar group, a carboxyl group, or combinations thereof;
each R^{α} independently is H or C₁-C₆ alkyl;
each R^{b} independently is halo, C₁₋₆ alkyl, an attachment site to at least one of the Drug units, or an attachment site to at least one of the Polar groups;
x is 0, 1, 2, 3 or 4;
y is 0, 1, 2 or 3;
R^{c} is a bond, —C(O)—, —S(O)—, —SO₂—, C₁₋₆ alkylene, C₁₋₆ alkynylene, triazolyl or combinations thereof; and
Y is a bond, —O—, —S—, —N(R^{a})—, —C(O)—, —S(O)—, —SO₂—C₁-C₆ alkylene, C₁-C₆ alkenylene, C₁-C₆ alkynylene, triazolyl, a group containing triazolyl, or combinations thereof.

In some embodiments, provided are Drug-Linker compounds, comprising a Linker compound described herein with at least one Drug unit attached via the attachment site.

In some embodiments, provided are Conjugates comprising a Targeting group attached to a Drug-Linker compound described herein.

In some embodiments, provided are pharmaceutical compositions comprising a Conjugate described herein and a pharmaceutically acceptable carrier.

In some embodiments, provided are methods of treating a subject in need thereof, comprising administering to the subject a conjugate described herein or a pharmaceutical composition described herein, wherein the subject has cancer or an autoimmune disease and the conjugate binds to a target antigen associated with the cancer or autoimmune disease.

These and other aspects of the present invention may be more fully understood by reference to the following detailed description, non-limiting examples of specific embodiments and the appended drawings.

### BRIEF DESCRIPTION OF THE DRAWINGS

FIG. 1 is a graph comparing tumor volume versus treatment time for in vivo tests of antitumor agents against OVCAR-3;

FIG. 2 is a graph comparing tumor volume versus treatment time for in vivo tests of antitumor agents against PA-1;

FIG. 3 is a graph comparing tumor volume versus treatment time for in vivo tests of antitumor agents against NCI-H292;

FIG. 4 is a graph comparing tumor volume versus treatment time for in vivo tests of antitumor agents against MDA-MB-468;

FIG. 5 is a graph comparing mean fluorescence intensity versus concentration for in vitro mAbs and ADCs binding to OVCAR-3 cells;

FIG. 6 is a graph comparing cell viability versus concentration for ADCs on OVCAR-3 cells;

FIG. 7 is a graph comparing tumor volume versus treatment time for in vivo tests of antitumor agents against NCI-H441;

FIG. 8 is a graph comparing tumor volume versus treatment time for in vivo tests of antitumor agents against HCC4006;

FIG. 9 is a graph comparing mean fluorescence intensity versus concentration for in vitro mAbs and ADCs binding to CFPAC-1 cells;

FIG. 10 is a graph comparing mean fluorescence intensity versus concentration for in vitro mAbs and ADCs binding to PC-3 cells;

FIG. 11 is a graph comparing viability versus concentration for ADCs on CFPAC-1;

FIG. 12 is a graph comparing viability versus concentration for ADCs on PC-3;

FIG. 13 is a graph comparing tumor volume versus treatment time for in vivo tests of antitumor agents against CFPAC-1; and

FIG. 14 is a graph comparing tumor volume versus treatment time for in vivo tests of antitumor agents against PC-3.

### DEFINITIONS

For convenience, certain terms in the specification, examples and claims are defined here. Unless stated otherwise, or implicit from context, the following terms and phrases have the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

As used herein and unless otherwise indicated, the terms “a” and “an” are taken to mean “one,” “at least one” or “one or more”. Unless otherwise required by context, singular terms used herein shall include pluralities and plural terms shall include the singular.

Unless the context requires otherwise, throughout the description and the claims, the words “comprise,” “comprising,” and the like are to be construed in an inclusive sense as opposed to an exclusive or exhaustive sense; that is to say, in the sense of “including, but not limited to”.

The terms “decreased,” “reduce,” “reduced,” “reduction,” “decrease,” and “inhibit” are all used herein generally to mean a decrease by a statistically significant amount relative to a reference.

The terms “increased,” “increase,” or “enhance,” or “activate” are all used herein to generally mean an increase by a statically significant amount relative to a reference.

As used herein, the terms “protein” and “polypeptide” are used interchangeably herein to designate a series of amino acid residues each connected to each other by peptide bonds between the alpha-amino and carboxyl groups of adjacent residues. The terms “protein” and “polypeptide” also refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to an encoded gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, an “epitope” refers to the amino acids conventionally bound by an immunoglobulin VHNL pair, such as the antibodies, antigen binding portions thereof and other binding agents described herein. Other binding agents comprise non-antibody scaffolds. An epitope can be formed on a polypeptide from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, about 9, or about 8-10 amino acids in a unique spatial conformation. An epitope defines the minimum binding site for an antibody, antigen binding portions thereof and other binding agent, and thus represents the target of specificity of an antibody, antigen binding portion thereof or other immunoglobulin-based binding agent. In the case of a single domain antibody, an epitope represents the unit of structure bound by a variable domain in isolation.

As used herein, “specifically binds” refers to the ability of a binding agent (e.g., an antibody or antigen binding portion thereof) described herein to bind to a target with a KD of 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. “Specifically binds” as stated herein also refers to the ability of a molecule (e.g., an antibody or antigen binding portion thereof or non-antibody scaffold) described herein to bind to a target with a KD of 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. Specific binding can be influenced by, for example, the affinity and avidity of the antibody, antigen binding portion or other binding agent and the concentration of target polypeptide. A person of ordinary skill in the art can determine appropriate conditions under which antibodies, antigen binding portions and other binding agents described herein selectively bind to a target molecule using any suitable methods, such as titration of an antibody or a binding agent in a suitable cell binding assay. A binding agent specifically bound to a target molecule is not displaced by a non-similar competitor. In certain embodiments, an antibody or antigen-binding portion thereof or other binding agent is said to specifically bind to a target molecule when it preferentially recognizes its target molecule in a complex mixture of proteins and/or macromolecules. Specific binding can be influenced by, for example, the affinity and avidity of the antibody, antigen binding portion or non-antibody scaffold and the concentration of target polypeptide. A person of ordinary skill in the art can determine appropriate conditions under which antibodies, antigen binding portions and non-antibody scaffolds described herein selectively bind to a target molecule using any suitable methods, such as titration of an antibody or a non-antibody scaffold in a suitable cell binding assay. A molecule specifically bound to a target molecule is not displaced by a non-similar competitor. In certain embodiments, an antibody or antigen-binding portion thereof or non-antibody scaffold is said to specifically bind to a target molecule when it preferentially recognizes its target molecule in a complex mixture of proteins and/or macromolecules.

Unless otherwise indicated, the term “alkyl” by itself or as part of another term refers to a substituted or unsubstituted straight chain or branched, saturated hydrocarbon having the indicated number of carbon atoms (e.g., “—C₁-C₅ alkyl,” “—C₁-C₅ alkyl,” or “—C₁-C₁₀” alkyl refer to an alkyl group having from 1 to 5, 1 to 8, or 1 to 10 carbon atoms, respectively). Examples include methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), and 3,3-dimethyl-2-butyl (—CH(CH₃)C(CH₃)₃.

Unless otherwise indicated, “alkenyl” by itself or as part of another term refers to a C₂-C₈ substituted or unsubstituted straight chain or branched, hydrocarbon with at least one site of unsaturation (i.e., a carbon-carbon, sp² double bond). Examples include, but are not limited to: ethylene or vinyl (—CH═CH₂), allyl (—CH₂CH═CH₂), cyclopentenyl (—C₅H₇), and 5-hexenyl (—CH₂CH₂CH₂CH₂CH═CH₂).

Unless otherwise indicated, “alkynyl” by itself or as part of another term refers to a refers to C₂-C₈, substituted or unsubstituted straight chain or branched, hydrocarbon with at least one site of unsaturation (i.e., a carbon-carbon, sp triple bond. Examples include, but are not limited to: acetylenic and propargyl.

Unless other indicated, “alkylene” refers to a saturated, branched or straight chain or hydrocarbon radical of 1-8 carbon atoms, and having two monovalent radical centers derived by the removal of two hydrogen atoms from the same or two different carbon atoms of a parent alkane. Typical alkylene radicals include, but are not limited to: methylene (—CH₂—), 1,2-ethyl (—CH₂CH₂—), 1,3-propyl (—CH₂CH₂CH₂—), 1,4-butyl (—CH₂CH₂CH₂CH₂—), and the like.

Unless otherwise indicated, “alkenylene” refers to an unsaturated, branched or straight chain hydrocarbon radical of 2-8 carbon atoms, and having two monovalent radical centers derived by the removal of two hydrogen atoms from the same or two different carbon atoms of a parent alkene. Typical alkenylene radicals include, but are not limited to: 1,2-ethylene (—CH═CH—).

Unless otherwise indicated, “alkynylene” refers to an unsaturated, branched or straight chain or cyclic hydrocarbon radical of 2-8 carbon atoms, and having two monovalent radical centers derived by the removal of two hydrogen atoms from the same or two different carbon atoms of a parent alkyne. Typical alkynylene radicals include, but are not limited to: acetylene, propargyl, and 4-pentynyl.

Unless otherwise indicated, the term “heteroalkyl,” by itself or in combination with another term, refers to a substituted or unsubstituted stable straight or branched chain hydrocarbon, or combinations thereof, saturated and from one to ten, preferably one to three, heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group (i.e., as part of the main chain) or at the position at which the alkyl group is attached to the remainder of the molecule. The heteroatom Si may be placed at any position of the heteroalkyl group, including the position at which the alkyl group is attached to the remainder of the molecule. Examples of heteroalkyl include the following: —CH₂CH₂OCH₃, —CH₂CH₂NHCH₃, —CH₂CH₂N(CH₃)CH₃, —CH₂SCH₂CH₃, CH₂CH₂S(O)CH₃, —CH₂CH₂S(O)₂CH₃, and —Si(CH₃)₃. Up to two heteroatoms may be consecutive, such as, for example, —CH₂NHOCH₃ and CH₂OSi(CH₃)₃. In some embodiments, a C₁ to C₄ heteroalkyl has 1 to 4 carbon atoms and 1 or 2 heteroatoms and a C₁ to C₃ heteroalkyl has 1 to 3 carbon atoms and 1 or 2 heteroatoms.

Unless otherwise indicated, the terms “heteroalkenyl” and “heteroalkynyl” by themselves or in combination with another term, refers to a substituted or unsubstituted stable straight or branched chain alkenyl or alkynyl having from one to ten, preferably one to three, heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of a heteroalkenyl or heteroalkynyl group (i.e., as part of the main chain) or at the position at which the alkyl group is attached to the remainder of the molecule. The heteroatom Si may be placed at any position of a heteroalkenyl or heteroalkynyl group, including the position at which the alkyl group is attached to the remainder of the molecule.

Unless otherwise indicated, the term “heteroalkylene” by itself or as part of another substituent refers to a substituted or unsubstituted divalent group derived from a heteroalkyl (as discussed above), as exemplified by —CH₂CH₂SCH₂CH₂— and —CH₂SCH₂CH₂NHCH₂—. In some embodiments, a C₁ to C₄ heteroalkylene has 1 to 4 carbon atoms and 1 or 2 heteroatoms and a C₁ to C₃ heteroalkylene has 1 to 3 carbon atoms and 1 or 2 heteroatoms. For heteroalkylene groups, heteroatoms can also occupy either or both of the chain termini. Still further, for alkylene and heteroalkylene linking groups, no orientation of the linking group is implied.

Unless otherwise indicated, the terms “heteroalkenylene” and “heteroalkynylene” by themselves or as part of another substituent refers to a substituted or unsubstituted divalent group derived from an heteroalkenyl or heteroalkynyl (as discussed above). In some embodiments, a C₂ to C₄ heteroalkenylene or heteroalkynylene has 1 to 4 carbon atoms. For heteroalkenylene and heteroalkynylene groups, heteroatoms can also occupy either or both of the chain termini. Still further, for alkylene and heteroalkenylene and heteroalkynylene linking groups, no orientation of the linking group is implied.

Unless otherwise indicated, a “C₃-C₈ carbocycle,” by itself or as part of another term, refers to a substituted or unsubstituted 3-, 4-, 5-, 6-, 7- or 8-membered monovalent, substituted or unsubstituted, saturated or unsaturated non-aromatic monocyclic or bicyclic carbocyclic ring derived by the removal of one hydrogen atom from a ring atom of a parent ring system. Representative —C₃-C₈carbocycles include, but are not limited to, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, and cyclooctadienyl.

Unless otherwise indicated, a “C₃-C₈ carbocyclo,” by itself or as part of another term, refers to a substituted or unsubstituted C₃-C₈ carbocycle group defined above wherein another of the carbocycle groups' hydrogen atoms is replaced with a bond (i.e., it is divalent).

Unless otherwise indicated, a “C₃-C₁₀ carbocycle,” by itself or as part of another term, refers to a substituted or unsubstituted 3-, 4-, 5-, 6-, 7-, 8-, 9- or 10-membered monovalent, substituted or unsubstituted, saturated or unsaturated non-aromatic monocyclic, bicyclic or tricyclic carbocyclic ring derived by the removal of one hydrogen atom from a ring atom of a parent ring system. Representative —C₃-C₁₀ carbocycles include, but are not limited to, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, and cyclooctadienyl. —C₃-C₁₀ carbocycles can further include fused cyclooctyne carbocycles, such as the fused cyclooctyne compounds disclosed in International Publication Number WO2011/136645 (the disclosure of which is incorporated by reference herein), including BCN (bicyclo[6.1.0]nonyne) and DBCO (Dibenzocyclooctyne).

Unless otherwise indicated, a “C₃-C₈ heterocycle,” by itself or as part of another term, refers to a substituted or unsubstituted monovalent substituted or unsubstituted aromatic or non-aromatic monocyclic or bicyclic ring system having from 3 to 8 carbon atoms (also referred to as ring members) and one to four heteroatom ring members independently selected from N, O, P or S, and derived by removal of one hydrogen atom from a ring atom of a parent ring system. One or more N, C or S atoms in the heterocycle can be oxidized. The ring that includes the heteroatom can be aromatic or nonaromatic. Unless otherwise noted, the heterocycle is attached to its pendant group at any heteroatom or carbon atom that results in a stable structure. Representative examples of a C₃-C₈ heterocycle include, but are not limited to, pyrrolidinyl, azetidinyl, piperidinyl, morpholinyl, tetrahydrofuranyl, tetrahydropyranyl, benzofuranyl, benzothiophene, indolyl, benzopyrazolyl, pyrrolyl, thiophenyl (thiophene), furanyl, thiazolyl, imidazolyl, pyrazolyl, pyrimidinyl, pyridinyl, pyrazinyl, pyridazinyl, isothiazolyl, and isoxazolyl. Unless otherwise indicate, the term “heterocarbocycle” is synonymous with the terms “heterocycle” or “heterocyclo” as described herein.

Unless otherwise indicated, “C₃-C₈ heterocyclo,” by itself or as part of another term, refers to a substituted or unsubstituted C₃-C₈ heterocycle group defined above wherein one of the heterocycle group's hydrogen atoms is replaced with a bond (i.e., it is divalent).

Unless otherwise indicated, “aryl” by itself or as part of another term, means a substituted or unsubstituted monovalent carbocyclic aromatic hydrocarbon radical of 6-20 carbon (preferably 6-14 carbon) atoms derived by the removal of one hydrogen atom from a single carbon atom of a parent aromatic ring system. Some aryl groups are represented in the exemplary structures as “Ar”. Typical aryl groups include, but are not limited to, radicals derived from benzene, substituted benzene, naphthalene, anthracene, biphenyl, and the like. An exemplary aryl group is a phenyl group.

Unless otherwise indicated, an “arylene” by itself or as part of another term, is an unsubstituted or substituted aryl group as defined above wherein one of the aryl group's hydrogen atoms is replaced with a bond (i.e., it is divalent) and can be in the ortho, meta, or para orientations.

Unless otherwise indicated, “heteroaryl” and “heterocycle” refer to a ring system in which one or more ring atoms is a heteroatom, e.g., nitrogen, oxygen, and sulfur. A heterocycle radical comprises 1 to 20 carbon atoms and 1 to 3 heteroatoms selected from N, O, P, and S. A heterocycle may be a monocycle having 3 to 7 ring members (2 to 6 carbon atoms and 1 to 3 heteroatoms selected from N, O, P, and S) or a bicycle having 7 to 10 ring members (4 to 9 carbon atoms and 1 to 3 heteroatoms selected from N, O, P, and S), for example: a bicyclo[4,5], [5,5], [5,6], or [6,6] system.

Unless otherwise indicated, an “heteroarylene” by itself or as part of another term, is an unsubstituted or substituted heteroaryl group as defined above wherein one of the heteroaryl group's hydrogen atoms is replaced with a bond (i.e., it is divalent).

Unless otherwise indicated, “carboxyl” refers to COOH or COO⁻M⁺, where M⁺ is a cation.

Unless otherwise indicated, “oxo” refers to (C═O).

Unless otherwise indicated, “substituted alkyl” and “substituted aryl” mean alkyl and aryl, respectively, in which one or more hydrogen atoms are each independently replaced with a substituent. Typical substituents include, but are not limited to, —X, —R¹⁰, —O—, —OR¹⁰, —SR¹⁰, —S⁻,—NR¹⁰₂, —NR¹⁰₃, =NR¹⁰, —CX₃, —CN, —OCN, —SCN, —N═C═O, —NCS, —NO, —NO₂, =N₂, —N₃, —NR¹⁰C(═O)R¹⁰, —C(═O)R¹⁰, —C(═O)NR¹⁰₂, —SO₃⁻, —SO₃H, —S(═O)₂R¹⁰, —OS(═O)₂OR¹⁰, —S(═O)₂NR¹⁰, —S(═O)R¹⁰, —OP(═O)(OR¹⁰)₂, —P(═O)(OR¹⁰)₂, —PO⁻₃, —PO₃H₂, -ASO₂H₂, —C(═O)R¹⁰, —C(═O)X, —C(═S)R¹⁰, —CO₂R¹⁰, —CO₂⁻, —C(═S)OR¹⁰, C(═O)SR¹⁰, C(═S)SR¹⁰, C(═O)NR¹⁰₂, C(═S)NR¹⁰₂, or C(═NR¹⁰)NR¹⁰₂, where each X is independently a halogen: —F, —Cl, —Br, or —I; and each R¹⁰ is independently —H, —C₁-C₂₀ alkyl, —C₆-C₂₀ aryl, —C₃-C₁₄ heterocycle, a protecting group or a prodrug moiety. Typical substitutents also include (═O). Alkylene, carbocycle, carbocyclo, arylene, heteroalkyl, heteroalkylene, heterocycle, and heterocyclo groups as described above may also be similarly substituted.

Unless otherwise indicated, “polyhydroxyl group” refers to an alkyl, alkylene, carbocycle or carbocyclo group including two or more, or three or more, substitutions of hydroxyl groups for hydrogen on carbon atoms of the carbon chain. In some embodiments, a polyhydroxyl group comprises at least three hydroxyl groups. In some embodiments, a polyhydroxyl group comprises carbon atoms containing only one hydroxyl group per carbon atom. A polyhydroxyl group may contain one or more carbon atoms that are not substituted with hydroxyl. A polyhydroxyl group may have each carbon atom substituted with a hydroxyl group. Examples of polyhydroxyl group includes linear (acyclic) or cyclic forms of monosaccharides such as C6 or C5 sugars, such as glucose, ribose, galactose, mannose, arabinose, 2-deoxyglucose, glyceraldehyde, erythrose, threose, xylose, lyxose, allose, altrose, gulose, idose, talose, aldose, and ketose, sugar acids such as gluconic acid, aldonic acid, uronic acid or ulosonic acid, and an amino sugars, such as glucosamine, N-acetyl glucosamine, galactosamine, and N-acetyl galactosamine. In some embodiments, polyhydroxyl group includes linear or cyclic forms of disaccharides and polysaccharides.

Unless otherwise indicated by context, “optionally substituted” refers to an alkyl, alkenyl, alkynyl, alkylaryl, arylalkyl heterocycle, aryl, heteroaryl, alkylheteroaryl, heteroarylalkyl, or other substituent, moiety or group as defined or disclosed herein wherein hydrogen atom(s) of that substituent, moiety or group has been optionally replaced with different moiety(ies) or group(s), or wherein an alicyclic carbon chain that comprise one of those substituents, moiety or group is interrupted by replacing carbon atom(s) of that chain with different moiety(ies) or group(s). In some aspects an alkene function group replaces two contiguous sp3 carbon atoms of an alkyl substituent, provided that the radical carbon of the alkyl moiety is not replaced, so that the optionally substituted alkyl is an unsaturated alkyl substituent.

Optional substituent replacing hydrogen(s) in any of the foregoing substituents, moieties or groups is independently selected from the group consisting of aryl, heteroaryl, hydroxyl, alkoxy, aryloxy, cyano, halogen, nitro, fluoroalkoxy, and amino, including mono-, di- and tri-substituted amino groups, and the protected derivatives thereof, or is selected from the group consisting of —X, —OR′, —SR′, —NH₂, —N(R′)(R″), —N(R″)₃, ═NR, —CX₃, —CN, —NO₂, —NR′C(═O)H, —NR′C(═O)R, —NR′C(═O)R″, —C(═O)R′, —C(═O)NH₂, —C(═O)N(R′)R″, —S(═O)₂R″, —S(═O)₂NH₂, —S(═O)₂N(R′)R″, —S(═O)₂NH₂, —S(═O)₂N(R′)R″, —S(═O)₂OR′, —S(═O)R″, —OP(═O)(OR′)(OR″), —OP(OH)₃, —P(═O)(OR′)(OR″), —PO₃H₂, —C(═O)R′, —C(═S)R′, —CO₂R′, —C(═S)OR″, —C(═O)SR′, —C(═S)SR′, —C(═S)NH₂, —C(═S)N(R′)(R″)₂, —C(═NR′)NH₂, —C(═NR′)N(R′)R″, and salts thereof, wherein each X is independently selected from the group consisting of a halogen: —F, —Cl, —Br, and —I; and wherein each R″ is independently selected from the group consisting of C₁-C₂₀ alkyl, C₂-C₂₀ alkenyl, C₂-C₂₀ alkynyl, C₆-C₂₄ aryl, C₃-C₂₄ heterocyclyl (including C₅-C₂₄ heteroaryl), a protecting group, and a prodrug moiety or two of R″ together with the heteroatom to which they are attached defines a heterocyclyl; and R′ is hydrogen or R″, wherein R″ is selected from the group consisting of C₁-C₂₀ alkyl, C₆-C₂₄ aryl, C₃-C₂₄ heterocyclyl (including C₅-C₂₄ heteroaryl), and a protecting group.

Typically, optional substituents are selected from the group consisting of —X, —OH, —OR″, —SH, —SR′, —NH₂, —NH(R″), —NR′(R″)₂, —N(R″)₃, ═NH, =NR″, —CX₃, —CN, —NO₂, —NR′C(═O)H, NR′C(═O)R″, —CO₂H, —C(═O)H, —C(═O)R″, —C(═O)NH₂, —C(═O)NR′R″— —S(═O)₂R″, —S(═O)₂NH₂, —S(═O)₂N(R′)R″, —S(═O)₂NH₂, —S(═O)₂N(R′)(R″), —S(═O)₂OR′, —S(═O)R″, —C(═S)R″, —C(═S)NH₂, —C(═S)N(R′)R″, —C(═NR′)N(R″)₂, and salts thereof, wherein each X is independently selected from the group consisting of —F and —Cl, R″ is typically selected from the group consisting of C₁-C₆ alkyl, C₆-C₁₀ aryl, C₃-C₁₀ heterocyclyl (including C₅-C₁₀ heteroaryl), and a protecting group; and R′ independently is hydrogen, C₁-C₆ alkyl, C₆-C₁₀ aryl, C₃-C₁₀ heterocyclyl (including C₅-C₁₀ heteroaryl), and a protecting group, independently selected from R″. More typically, substituents are selected from the group consisting of —X, —R″, —OH, —OR″, —NH₂, —NH(R″), —N(R″)₂, —N(R″)₃, —CX₃, —NO₂, —NHC(═O)H, —NHC(═O)R″, —C(═O)NH₂, —C(═O)NHR″, —C(═O)N(R″)₂, —CO₂H, —CO₂R″, —C(═O)H, —C(═O)R″, —C(═O)NH₂, —C(═O)NH(R″), —C(═O)N(R″)₂, —C(═NR′)NH₂, —C(═NR′)NH(R″), —C(═NR′)N(R″)₂, a protecting group and salts thereof, wherein each X is —F, R″ is independently selected from the group consisting of C₁-C₆ alkyl, C₆-C₁₀ aryl, C₅-C₁₀ heteroaryl and a protecting group; and R′ is selected from the group consisting of hydrogen, C₁—C alkyl and a protecting group, independently selected from R″.

The compounds of the invention, or their pharmaceutically acceptable salts may contain one or more asymmetric centers and may thus give rise to enantiomers, diastereomers, and other stereoisomeric forms that are defined, in terms of absolute stereochemistry, as (R) or (S) or, as (D) or (L) for amino acids. The present invention is meant to include all such possible isomers, as well as their racemic and optically pure forms. Optically active (+) and ( ), (R) and (S), or (D) and (L) isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centres of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers,” which refers to two stereoisomers whose molecules are nonsuperimposeable mirror images of one another. The present invention also includes “diastereomers,” which refers to two or more stereoisomers of a compound that have different configurations at one or more of the equivalent stereocenters and are not mirror images of each other.

Although structures shown throughout the specification are depicted with specific stereocenters, the specification should be read to include variations in those stereocenters. For example, the structure of exatecan may be shown in the (S,S) configuration, but the (R,S) diastereomer of exatecan is also envisioned as being found in a separate embodiment of a conjugate as described herein.

Unless otherwise indicated, the term “Drug unit” or drug refers to cytotoxic agents (such as chemotherapeutic agents or drugs), immunomodulatory agents, nucleic acids (including siRNAs), growth inhibitory agents, toxins (e.g., protein toxins, enzymatically active toxins of bacterial, fungal, plant, or animal origin, or fragments thereof), radioactive isotopes, PROTACs and other compounds that are active against target cells when delivered to those cells.

Unless otherwise indicated, the term “Polymer unit” refers to a polymeric moiety composed of repeating subunits. Examples of polymer units include polyamides and polyethers. In some embodiments, the Polymer unit is selected from an optionally substituted polyamide, a substituted polyether, or combinations thereof. In further embodiments, the Polymer unit is selected from
(i) an optionally substituted polyamide comprising the formula

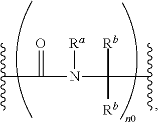

or a stereoisomer thereof, wherein each R^{a} is independently H or C₁₋₆ alkyl and each R^{b} is independently H or C₁₋₆ alkyl, and n⁰ is independently 2-26;
(ii) a substituted polyether comprising the formula

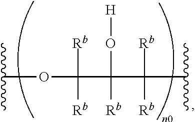

or a stereoisomer thereof, wherein each R^{b} is independently H or C₁₋₆ alkyl, and n⁰ is independently 2-26; or
(iii) combinations thereof.

Unless otherwise indicated, the term “Sugar unit” or “sugar group” refers to a carbohydrate group. Examples of sugar units include glycosides.

Unless otherwise indicated, the term “Carboxyl unit” or “carboxyl group” refers to a group including a carbonyl group [—C(O)—], a carboxyl group [—CO₂H], and/or a carboxylate group [—CO₂M, M refers to a cationic counterion].

Unless otherwise indicated, the term “Stretcher group” refers to a linking moiety that connects the Targeting group to the enzyme-cleavable group.

Unless otherwise indicated, the term “polyamide” refers to polymeric groups composed of repeating subunits containing amide bonds.

Unless otherwise indicated, the term “polyether” refers to polymeric groups composed to repeating subunits containing ether bonds.

Unless otherwise indicated, the term “enzyme-cleavable group” refers to a group that is cleavable by the action of a metabolic process or reaction inside a cell or in the extracellular milieu, whereby the covalent attachment between a Drug unit (e.g., a cytotoxic agent) and the Linker unit or portion thereof is broken, resulting in the free Drug unit, or a metabolite of the Linker unit-Drug, which is dissociated from the remainder of the Linker unit.

Unless otherwise indicated, the term “Targeting group” refers to a macromolecule, such a protein, polypeptide or peptide, that specifically binds to a target molecule. Examples of targeting groups include antibodies.

The phrase “pharmaceutically acceptable salt,” as used herein, refers to pharmaceutically acceptable organic or inorganic salts of a compound (e.g., a Linker, Drug Linker, or a conjugate). The compound typically contains at least one amino group, and accordingly acid addition salts can be formed with this amino group. Exemplary salts include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, linleate, gentisinate, fumarate, gluconate, glucuronate, saccharate, formate, benzoate, glutamate, methanesulfonate, ethanesulfonate, benzenesulfonate, toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

As used herein, the term “consisting essentially of” refers to those elements required for a given embodiment. The term permits the presence of elements that do not materially affect the basic and novel or functional characteristic(s) of that embodiment.

As used herein, the term “consisting of” refers to compositions, methods, and respective components thereof as described herein, which are exclusive of any element not recited in that description of the embodiment.

Other than in the examples, or where otherwise indicated, all numbers expressing quantities of ingredients or reaction conditions used herein should be understood as modified in all instances by the term “about.” The term “about” when used in connection with percentages can mean+/−1%.

The terms “statistically significant” or “significantly” refer to statistical significance and generally mean a two standard deviation (2SD) difference, above or below a reference value.

Other terms are defined herein within the description of the various aspects of the invention.

### DETAILED DESCRIPTION

Provided herein are Linker compounds comprising a Linker unit, at least one Polar group, and optionally at least one of an enzyme-cleavable group and a Stretcher group. Such Linker compounds have hydrophilic characteristics that maintain the intrinsic properties of antibodies conjugated with the Linkers and drugs. In particular, the Linkers aid in maintaining the hydrophilic properties of the antibodies when conjugated at higher drug loading and/or to hydrophobic drugs and other agents.

In some embodiments, provided are a Linker compound, or a stereoisomer or salt thereof, comprising:
(a) a Linker unit having from 1 to 4 attachment sites for a Drug unit and having one of the following structures (i) or (ii):

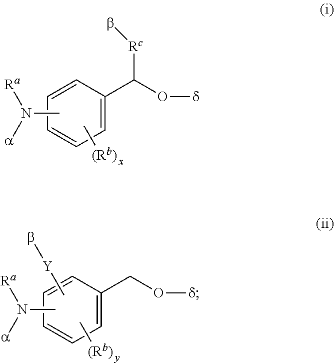

(b) at least one Polar group comprising a Polymer unit, optionally a Sugar unit, optionally a Carboxyl unit, and combinations thereof; and
(c) optionally a Stretcher group having an attachment site for a Targeting group; wherein:
α— is an attachment site to an enzyme-cleavable group;
β— is an attachment site to the at least one Polar group;
δ— is H, an attachment site to at least one of the Drug units, or an attachment site to a linking group attached to the at least one of the Drug units;
the Polymer unit comprises a polyamide, a polyether, or a combination thereof, wherein the polyether comprises a hydroxyl group, a polyhydroxyl group, a sugar group, a carboxyl group, or combinations thereof;
each R^{a} independently is H or C₁-C₆ alkyl;
each R^{b} independently is halo, C₁₋₆ alkyl, an attachment site to at least one of the Drug units, or an attachment site to at least one of the Polar groups;
x is 0, 1, 2, 3 or 4;
y is 0, 1, 2 or 3;
R^{c} is a bond, —C(O)—, —S(O)—, —SO₂—, C₁₋₆ alkylene, C₁₋₆ alkynylene, triazolyl or combinations thereof; and
   Y is a bond, —O—, —S—, —N(R^{a})—, —C(O)—, —S(O)—, —SO₂—C₁-C₆ alkylene, C₁-C₆ alkenylene, C₁-C₆ alkynylene, a group containing triazolyl, or combinations thereof.

In some embodiments, the Linker unit has one of the following structures (i-a), or (ii-a), or (iii-a).

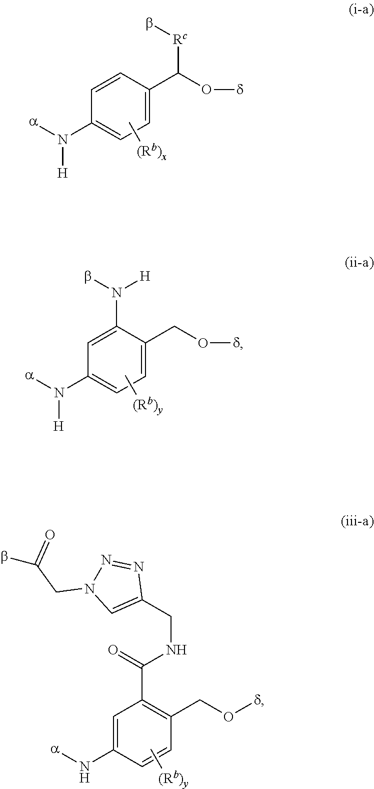

or a stereoisomer or salt thereof.

In some embodiments, wherein the Linker unit has one of the following structures (i-b), (i-c), (i-d), (i-e) or (i-f):

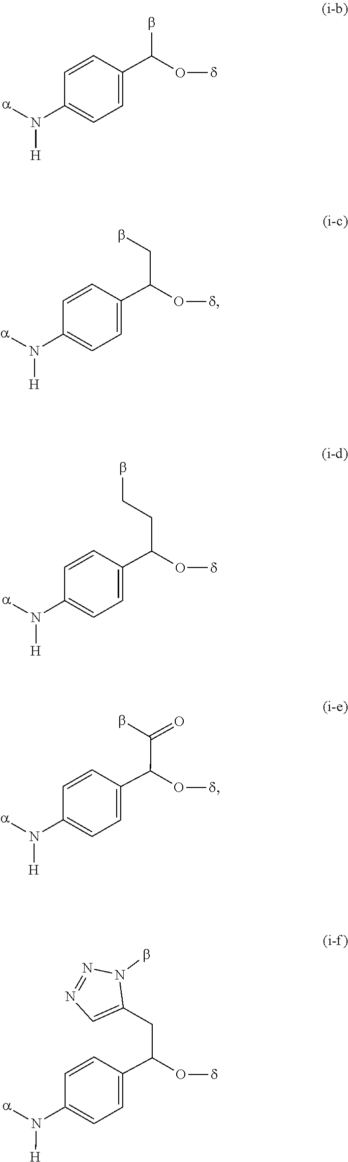

or a stereoisomer or salt thereof.

In some embodiments, the Linker unit has the following structure (ii-b) or (iii-b):

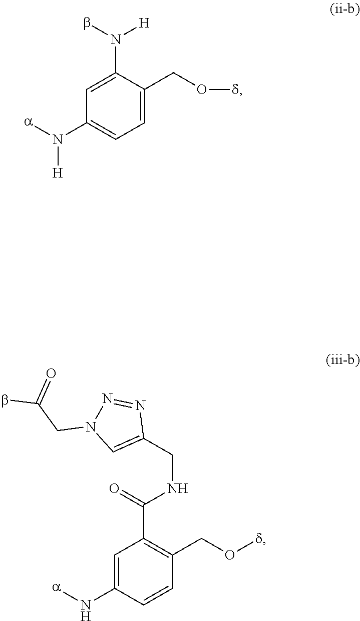

or a stereoisomer or salt thereof.

### Polar Groups

In some embodiments, provided are Linker compounds as described above, or stereoisomers or salts thereof, wherein the at least one Polar group comprises at least one Sugar unit having the following formula:

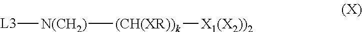

or a stereoisomer or salt thereof, wherein: each X is independently selected from NH and O; each R is independently selected from hydrogen, acetyl, a monosaccharide, a disaccharide, and a polysaccharide; each X₁ is independently selected from CH₂ and C(O); each X₂ is independently selected from H, OH and OR; k is 1 to 10; and L3 is a point of attachment to a remainder of the Polar group.

In some embodiments, the at least one Sugar unit described above has one of the following structures (XII) or (XIII):

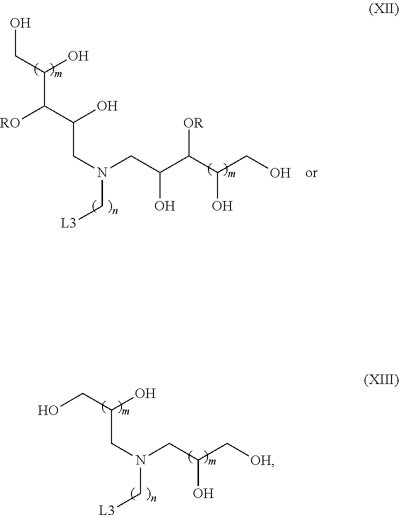

or a stereoisomer or salt thereof, wherein: each R is independently selected from hydrogen, a monosaccharide, a disaccharide and a polysaccharide; m is 1 to 8; and n is 0 to 4.

In some embodiments, provided are Linker compounds as described above, or stereoisomers or salts thereof, comprising a Polar group having a formula selected from:

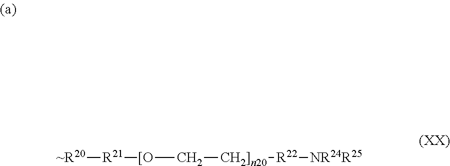

or a stereoisomer a salt thereof, wherein: R²⁰ is an attachment group to site R^{b}, or to the enzyme-cleavable group; R²¹ and R²² are each, independently, a bond or C₁-C₃ alkylene; R²⁴ and R²⁵ are each independently selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; and —C(O)—R²⁸, where R²⁸ is a Sugar unit of formula (XII) or (XIII); or —NR²⁴R²⁵ together from a C₃-C₈ heterocycle, provided that R²⁴ and R²⁵ are not both H; and n20 is 2 to 26; or

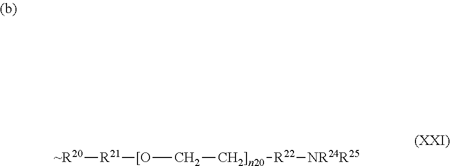

or a stereoisomer or salt thereof, wherein: R²⁰ is an attachment group to site R^{b}, or to the enzyme-cleavable group; R²¹ and R²² are each, independently, a bond or C₁-C₃ alkylene; one of R²⁴ and R²⁵ is selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; and —C(O)—R²⁸, where R²⁸ is a Sugar unit of formula (XII) or (XIII); and the other of R²⁴ and R²⁵ is a polyethylene glycol, optionally having 1 to 24 ethylene glycol subunits; and n20 is 2 to 26; or

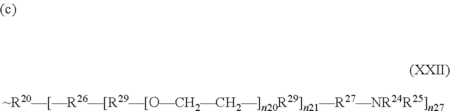

or a stereoisomer or salt thereof, wherein: R²⁰ is an attachment group to site R^{b}, or to the enzyme-cleavable group; R²⁶ and R²⁷ are each optional and are, independently, selected from a bond, C₁-C₁₂ alkylene, —NH—C₁-C₁₂ alkylene, —C₁-C₁₂ alkylene-NH—, —C₁-C₁₂ alkylene-N(CH₃)—, —C(O)—C₁-C₁₂ alkylene, —C₁-C₁₂ alkylene-C(O)—, —NH—C₁-C₁₂ alkylene-C(O)— and —C(O)—C₁-C₁₂ alkylene-NH—; one of R²⁴ and R²⁵ is selected from a H; polyhydroxyl group; substituted polyhydroxyl group; —C(O)— polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; and —C(O)—R²⁸, where R²⁸ is a Sugar unit of formula (XII) or (XIII); and the other of R²⁴ and R²⁵ is selected from H; polyhydroxyl group; substituted polyhydroxyl group; —C(O)-polyhydroxyl group; substituted —C(O)— polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; and —C(O)—R²⁸, where R²⁸ is a Sugar unit of formula (XII) or (XIII); and polyethylene glycol, optionally having 1 to 24 ethylene glycol subunits; or —NR²⁴R²⁵ together from a C₃-C₈ heterocycle, provided that R²⁴ and R²⁵ are not both H; each R²⁹ is optional and independently selected from —C(O)—, —NH—, —C(O)—C₁-C₆ alkylene-, —NH—C₁-C₆ alkylene-, —C₁-C₆ alkylene-NH—, —C₁-C₆ alkylene-C(O)—, —NH(CO)—C₁-C₆alkylene-, —N(CH₃)—(CO)—C₁-C₆alkylene-, —NH(CO)NH—, and triazole; n20 is 2 to 26; n21 is 1 to 4; and n27 is 1 to 4, or

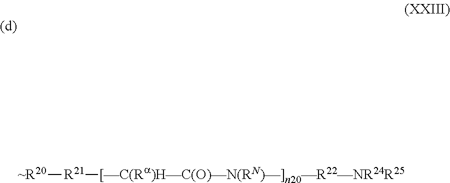

or a stereoisomer or salt thereof, wherein: R²⁰ is an attachment group to site R^{b}, or to the enzyme-cleavable group; R²¹ is a bond, C₁-C₃ alkylene, —C₁-C₃alkylene-[O—CH₂—CH₂-]ₙ₂₀, —[CH₂—CH₂—O]ₙ₂₀—C₁-C₃alkylene- or —C₁-C₃alkylene-[O—CH₂—CH₂-]ₙ₂₀-C(O)—; R²² is C₁-C₃ alkylene, —C₁-C₃alkylene-[O—CH₂—CH₂-]ₙ₂₀, —[CH₂—CH₂—O]ₙ₂₀—C₁-C₃alkylene- or —C₁-C₃alkylene-[O—CH₂—CH₂-]ₙ₂₀-C(O)—; each R^{α} is independently H or —R²²—NR²⁴R²⁵; each R^{N} is independently H, C₁-C₆ alkyl or —R²²—NR²⁴R²⁵; R²⁴ and R²⁵ are each independently selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; and —C(O)—R²⁸, where R²⁸ is a Sugar unit of formula (XII) or (XIII); or —NR²⁴R²⁵ together from a C₃-C₈ heterocycle, provided that R²⁴ and R²⁵ are not both H; and each n20 is independently 2 to 26, or

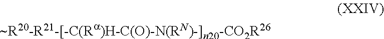

or a stereoisomer or salt thereof, wherein: R²⁰ is an attachment group to site R^{b}, or to the enzyme-cleavable group; R²¹ and R²² are each, independently, a bond, C₁-C₃ alkylene, or —C₁-C₃alkylene[O—CH₂—CH₂-]ₙ₂₀; each R^{α} is independently H or —R²²—NR²⁴R²⁵; each R^{N} is independently H, C₁-C₆ alkyl or —R²²—NR²⁴R²⁵; R²⁴ and R²⁵ are each independently selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; and —C(O)—R²⁸, where R²⁸ is a Sugar unit of formula (XII) or (XIII); or —NR²⁴R²⁵ together from a C₃-C₈ heterocycle, provided that R²⁴ and R²⁵ are not both H; R²⁶ is H or C₁-C₄ alkyl; and each n20 is independently 2 to 26, with the proviso that at least one R^{a} or R^{N} is —R²²—NR²⁴R²⁵; or

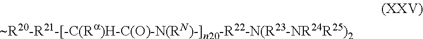

or a stereoisomer or salt thereof, wherein: R²⁰ is an attachment group to site R^{b}, or to the enzyme-cleavable group; R²¹ and R²² are each, independently, a bond, C₁-C₃ alkylene, or —C₁-C₃alkylene-[O—CH₂—CH₂-]ₙ₂₀; each R^{α} is independently H or —R²²—NR²⁴R²⁵; each R^{N} is independently H or C₁-C₆ alkyl; each R²³ is independently C₁-C₆ alkylene; R²⁴ and R²⁵ are each independently selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; and —C(O)—R²⁸, where R²⁸ is a Sugar unit of formula (XII) or (XIII); or —NR²⁴R²⁵ together from a C₃-C₈ heterocycle, provided that R²⁴ and R²⁵ are not both H; and each n20 is independently 2 to 26.

In some embodiments, provided are Linker compounds as described above, comprising a Polar group having a formula selected from:

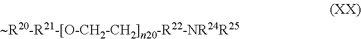

or a stereoisomer a salt thereof, wherein: R²⁰ is an attachment group to site R^{b}, or to the enzyme-cleavable group; R²¹ and R²² are each, independently, a bond or C₁-C₃ alkylene; R²⁴ and R²⁵ are each independently selected from a H; polyhydroxyl group; substituted polyhydroxyl group; —C(O)-polyhydroxyl group; substituted —C(O)-polyhydroxyl group; substituted —C₁-C₈ alkyl; substituted —C(O)—C₁-C₈ alkyl; a chelator; and —C(O)—R²⁸, where R²⁸ is a Sugar unit of formula (XII) or (XIII), provided that R²⁴ and R²⁵ are not both H; and n20 is 2 to 26; or

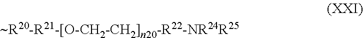

or a stereoisomer or salt thereof, wherein: R²⁰ is an attachment group to site R^{b}, or to the enzyme-cleavable group; R²¹ and R²² are each, independently, a bond or C₁-C₃ alkylene; one of R²⁴ and R²⁵ is selected from a H; polyhydroxyl group; substituted polyhydroxyl group; —C(O)-polyhydroxyl group; substituted —C(O)-polyhydroxyl group; substituted —C₁-C₈ alkyl; substituted —C(O)—C₁-C₈ alkyl; a chelator; and —C(O)—R²⁸, where R²⁸ is a Sugar unit of formula (XII) or (XIII); and the other of R²⁴ and R²⁵ is a polyethylene glycol, optionally having 1 to 24 ethylene glycol subunits; and n20 is 2 to 26;

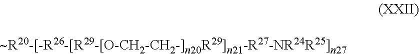

or a stereoisomer or salt thereof, wherein: R²⁰ is an attachment group to site R^{b}, or to the enzyme-cleavable group; R²⁶ and R²⁷ are each optional and are, independently, selected from a bond, C₁-C₁₂ alkylene, —NH—C₁-C₁₂ alkylene, —C₁-C₁₂ alkylene-NH—, —C₁-C₁₂ alkylene-N(CH₃)—, —C(O)—C₁-C₁₂ alkylene, —C₁-C₁₂ alkylene-C(O)—, —NH—C₁-C₁₂ alkylene-C(O)— and —C(O)—C₁-C₁₂ alkylene-NH—; one of R²⁴ and R²⁵ is selected from a H; polyhydroxyl group; substituted polyhydroxyl group; —C(O)— polyhydroxyl group; substituted —C(O)-polyhydroxyl group; substituted —C₁-C₈ alkyl; substituted —C(O)—C₁-C₈ alkyl; a chelator; and —C(O)—R²⁸, where R²⁸ is a Sugar unit of formula (XII) or (XIII); and the other of R²⁴ and R²⁵ is selected from H; polyhydroxyl group; substituted polyhydroxyl group; —C(O)-polyhydroxyl group; substituted —C(O)-polyhydroxyl group; substituted —C₁-C₈ alkyl; substituted —C(O)—C₁-C₈ alkyl; a chelator; and —C(O)—R²⁸, where R²⁸ is a Sugar unit of formula (XII) or (XIII); and polyethylene glycol, optionally having 1 to 24 ethylene glycol subunits, provided that R²⁴ and R²⁵ are not both H; each R²⁹ is optional and independently selected from —C(O)—, —NH—, —C(O)—C₁-C₆ alkylene-, —NH—C₁-C₆ alkylene-, —C₁-C₆ alkylene-NH—, —C₁-C₆ alkylene-C(O)—, —NH(CO)—C₁-C₆alkylene-, —N(CH₃)—(CO)—C₁-C₆alkylene-, —NH(CO)NH—, and triazole; n20 is 2 to 26; n21 is 1 to 4; and n27 is 1 to 4, or

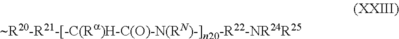

or a stereoisomer or salt thereof, wherein: R²⁰ is an attachment group to site R^{b}, or to the enzyme-cleavable group; R²¹ is a bond, C₁-C₃ alkylene, —C₁-C₃alkylene-[O—CH₂—CH₂-]ₙ₂₀, —[CH₂—CH₂—O]ₙ₂₀—C₁-C₃alkylene- or —C₁-C₃alkylene-[O—CH₂—CH₂-]ₙ₂₀-C(O)—; R²² is C₁-C₃ alkylene, —C₁-C₃alkylene-[O—CH₂—CH₂-]ₙ₂₀, —[CH₂—CH₂—O]ₙ₂₀—C₁-C₃alkylene- or —C₁-C₃alkylene-[O—CH₂—CH₂-]ₙ₂₀-C(O)—; each R^{α} is independently H or —R²²—NR²⁴R²⁵; each R^{N} is independently H, C₁-C₆ alkyl or —R²²—NR²⁴R²⁵; R²⁴ and R²⁵ are each independently selected from a H; polyhydroxyl group; substituted polyhydroxyl group; —C(O)-polyhydroxyl group; substituted —C(O)-polyhydroxyl group; substituted —C₁-C₈ alkyl; substituted —C(O)—C₁-C₈ alkyl; a chelator; and —C(O)—R²⁸, where R²⁸ is a Sugar unit of formula (XII) or (XIII), provided that R²⁴ and R²⁵ are not both H; and each n20 is independently 2 to 26, or

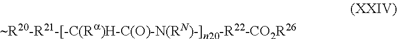

or a stereoisomer or salt thereof, wherein: R²⁰ is an attachment group to site R^{b}, or to the enzyme-cleavable group; R²¹ and R²² are each, independently, a bond, C₁-C₃ alkylene, or —C₁-C₃alkylene[O—CH₂—CH₂-]ₙ₂₀; each R^{α} is independently H or —R²²—NR²⁴R²⁵; each R^{N} is independently H, C₁-C₆ alkyl or —R²²—NR²⁴R²⁵; R²⁴ and R²⁵ are each independently selected from a H; polyhydroxyl group; substituted polyhydroxyl group; —C(O)-polyhydroxyl group; substituted —C(O)-polyhydroxyl group; substituted —C₁-C₈ alkyl; substituted —C(O)—C₁-C₈ alkyl; a chelator; and —C(O)—R²⁸, where R²⁸ is a Sugar unit of formula (XII) or (XIII), provided that R²⁴ and R²⁵ are not both H; R²⁶ is H or C₁-C₄ alkyl; and each n20 is independently 2 to 26, with the proviso that at least one R⁰ or R^{N} is —R²²—NR²⁴R²⁵; or

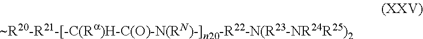

or a stereoisomer or salt thereof, wherein: R²⁰ is an attachment group to site R^{b}, or to the enzyme-cleavable group; R²¹ and R²² are each, independently, a bond, C₁-C₃ alkylene, or —C₁-C₃alkylene-[O—CH₂—CH₂-]ₙ₂₀; each R^{α} is independently H or —R²²—NR²⁴R²⁵; each R^{N} is independently H or C₁-C₆ alkyl; each R²³ is independently C₁-C₆ alkylene; R²⁴ and R²⁵ are each independently selected from a H; polyhydroxyl group; substituted polyhydroxyl group; —C(O)-polyhydroxyl group; substituted —C(O)-polyhydroxyl group; substituted —C₁-C₈ alkyl; substituted —C(O)—C₁-C₈ alkyl; a chelator; and —C(O)—R²⁸, where R²⁸ is a Sugar unit of formula (XII) or (XIII), provided that R²⁴ and R²⁵ are not both H; and each n20 is independently 2 to 26.

In some embodiments, the Linker compound comprises a Polar group wherein both R²⁴ and R²⁵ are not H. For example, in some embodiments R²⁴ and R²⁵ are each independently selected from H and a polyhydroxyl group, provided that R²⁴ and R²⁵ are not both H.

In some embodiments, wherein the polyhydroxyl group as described above is a linear monosaccharide, optionally selected from a C6 or C5 sugar, a sugar acid and an amino sugar. In more specific embodiments, the polyhydroxyl group is a the C6 or C5 sugar is selected from glucose, ribose, galactose, mannose, arabinose, 2-deoxyglucose, glyceraldehyde, erythrose, threose, xylose, lyxose, allose, altrose, gulose, idose, talose, aldose, and ketose; or the polyhydroxyl group is a sugar acid is selected from gluconic acid, aldonic acid, uronic acid and ulosonic acid; or the polyhydroxyl group is an amino sugar is selected from glucosamine, N-acetyl glucosamine, galactosamine, and N-acetyl galactosamine.

In some embodiments, provided are Linker compounds comprising a Polar group selected from the following, or a stereoisomer or salt thereof:

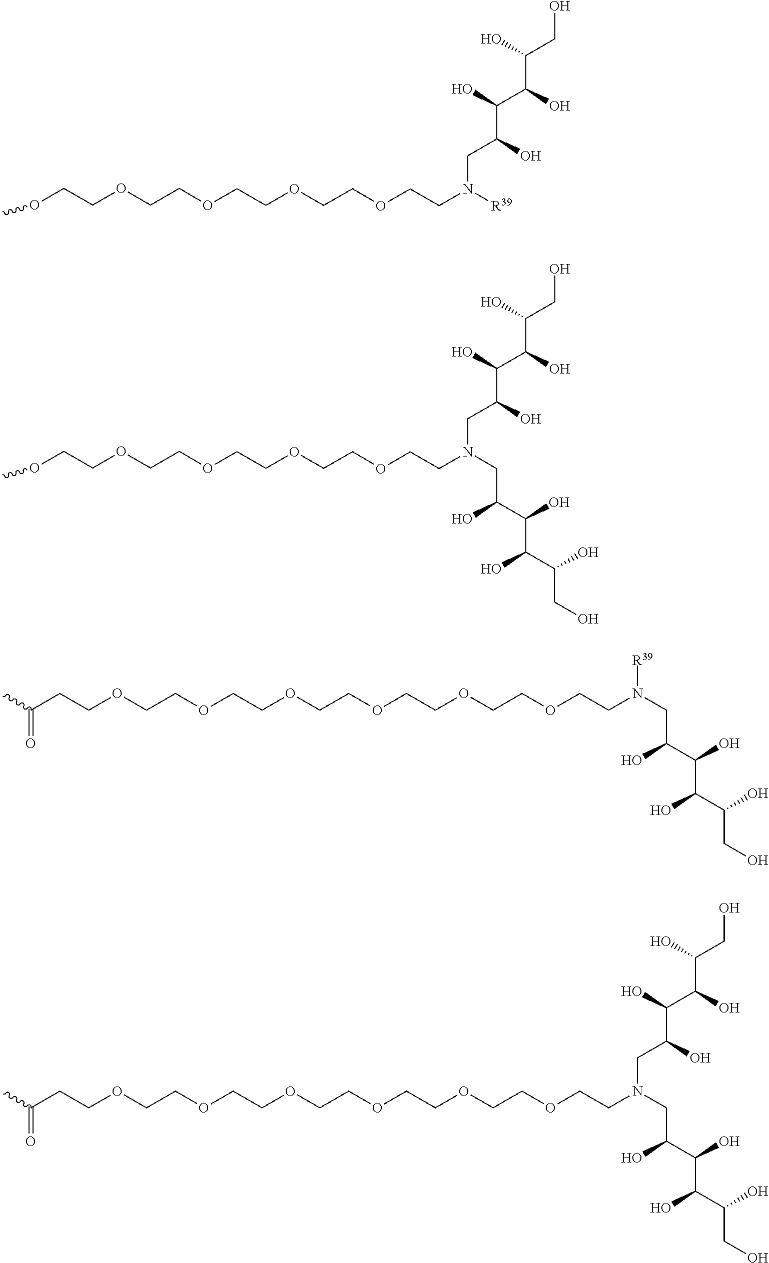

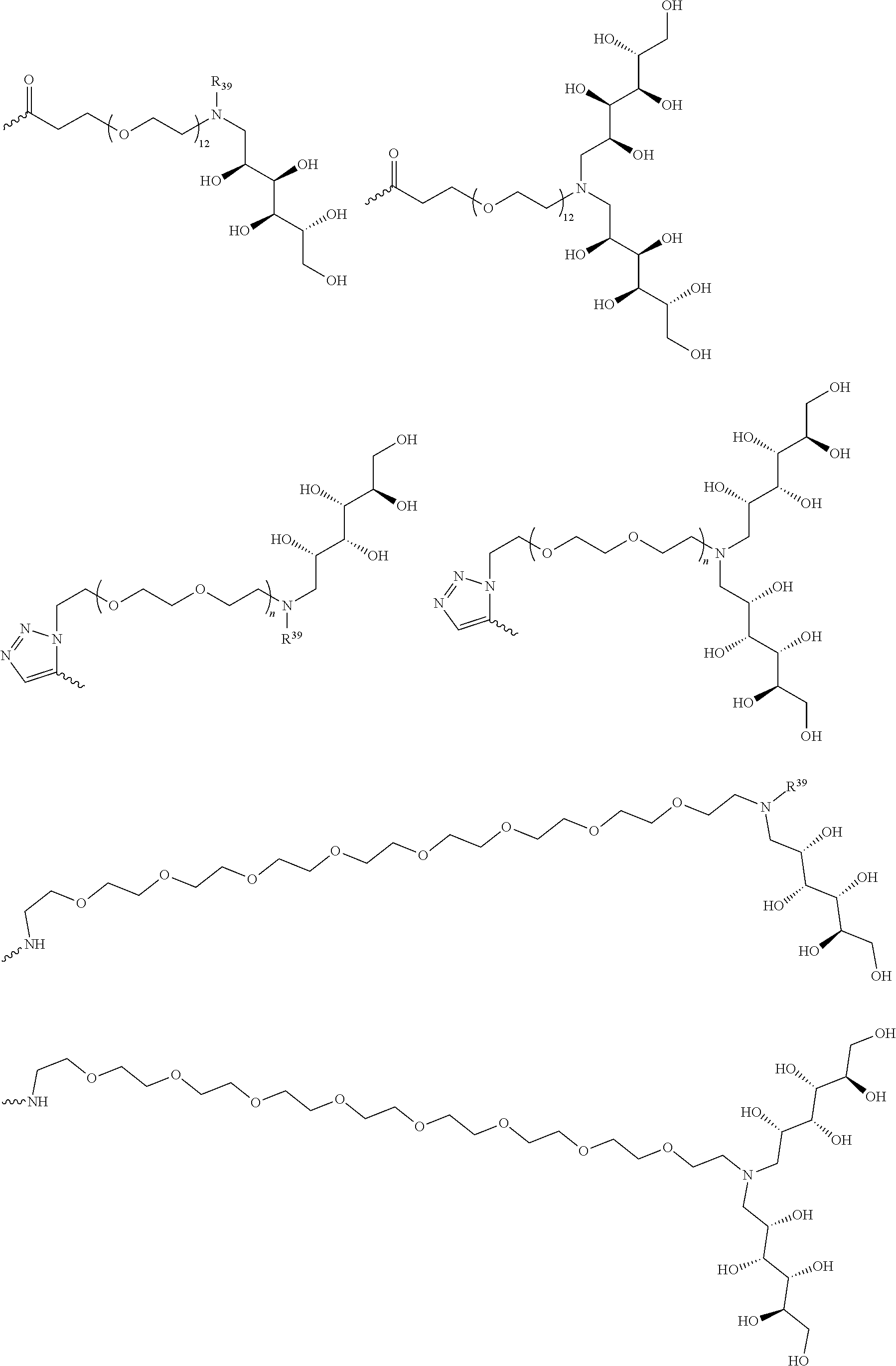

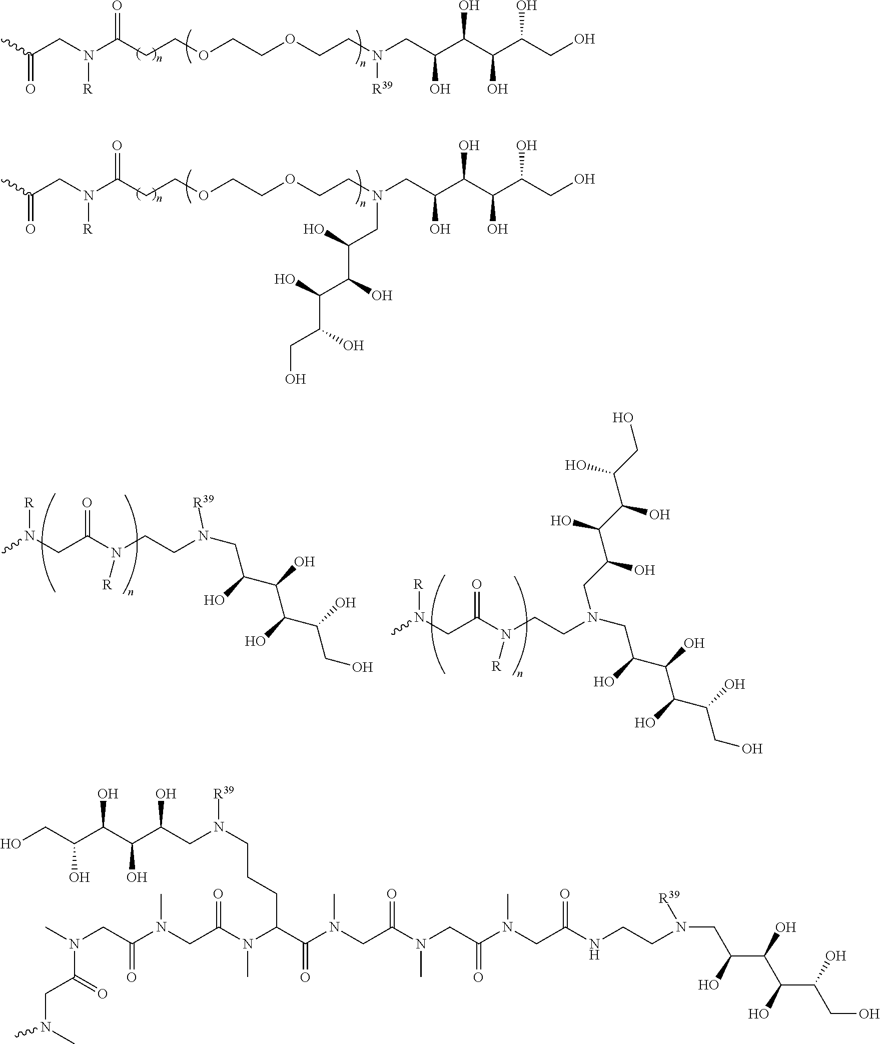

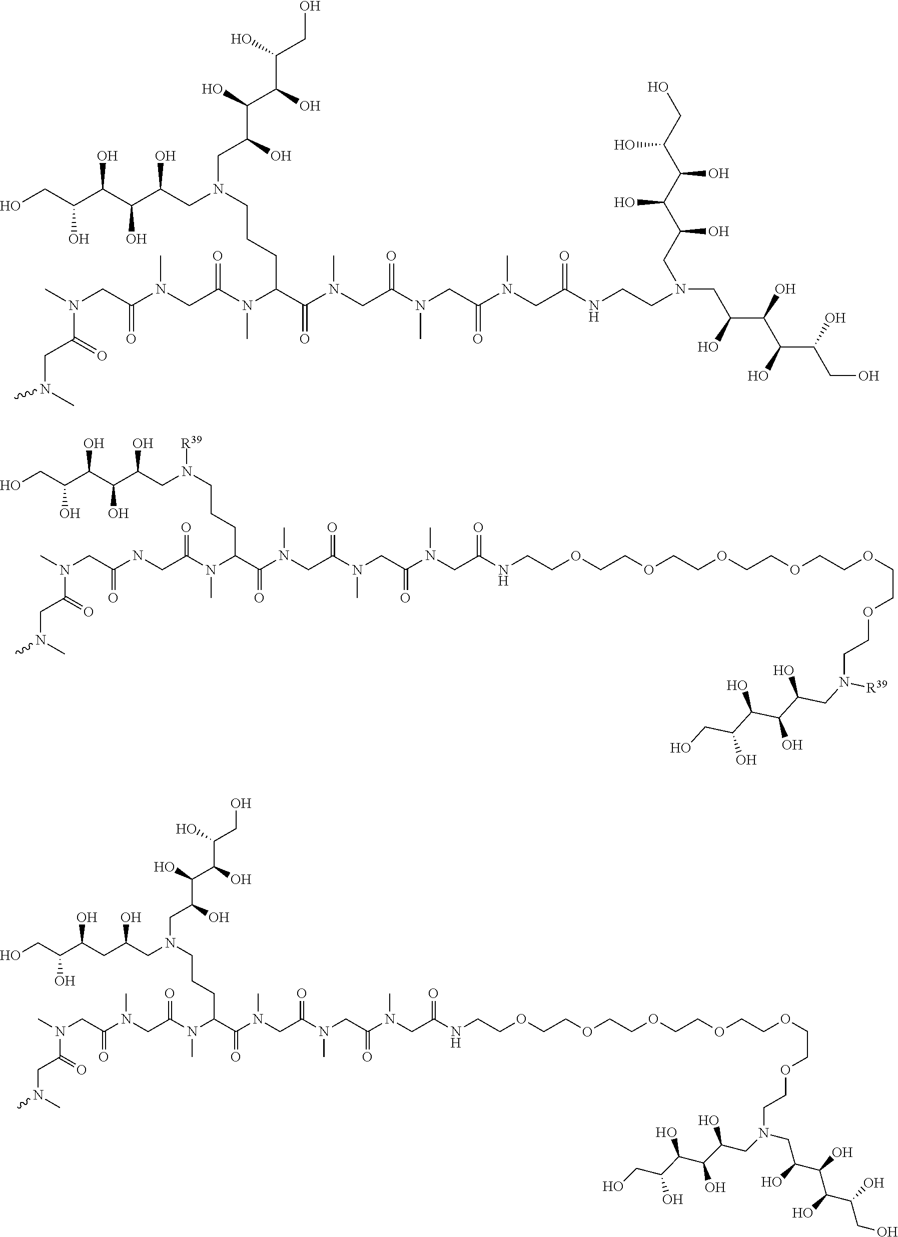

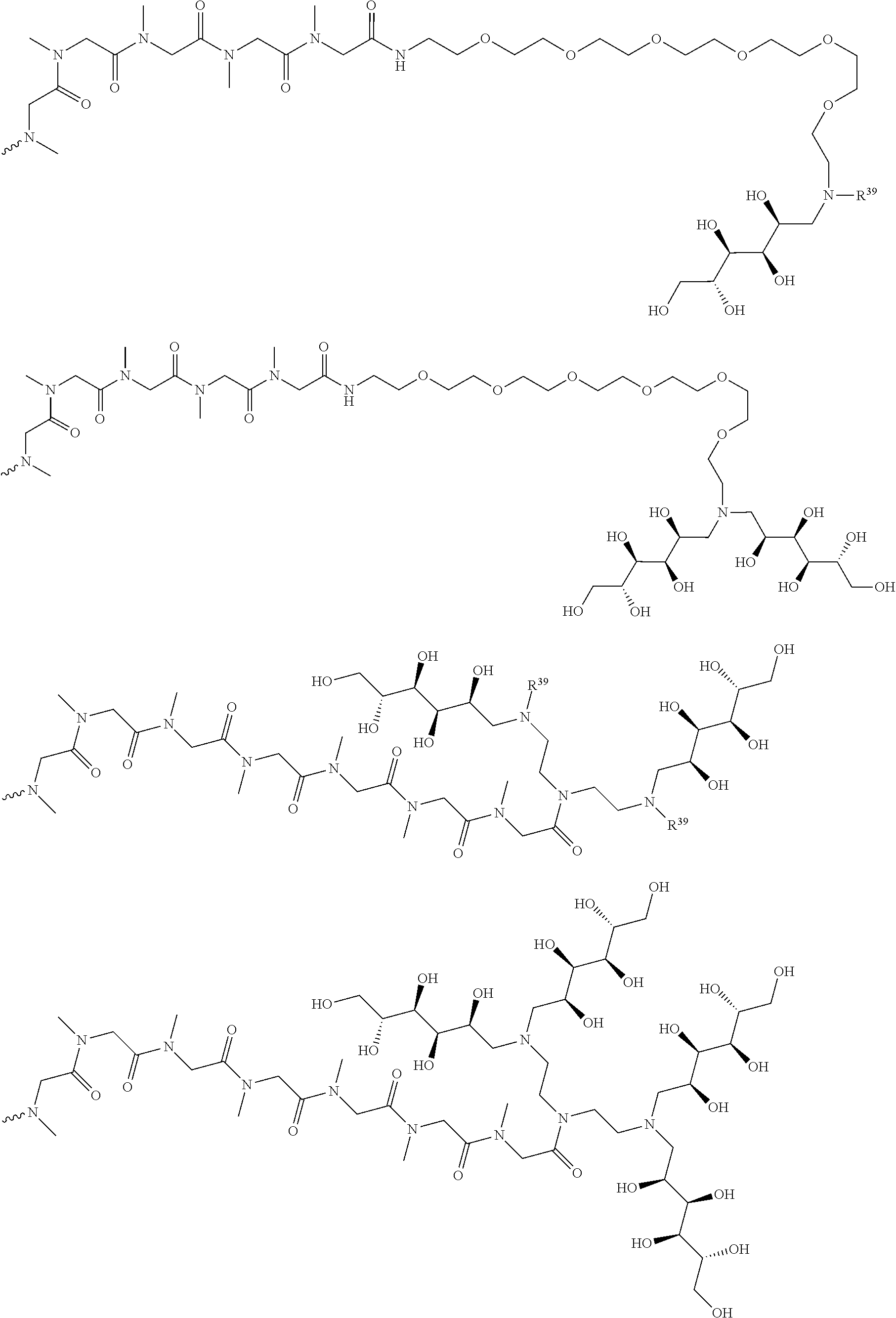

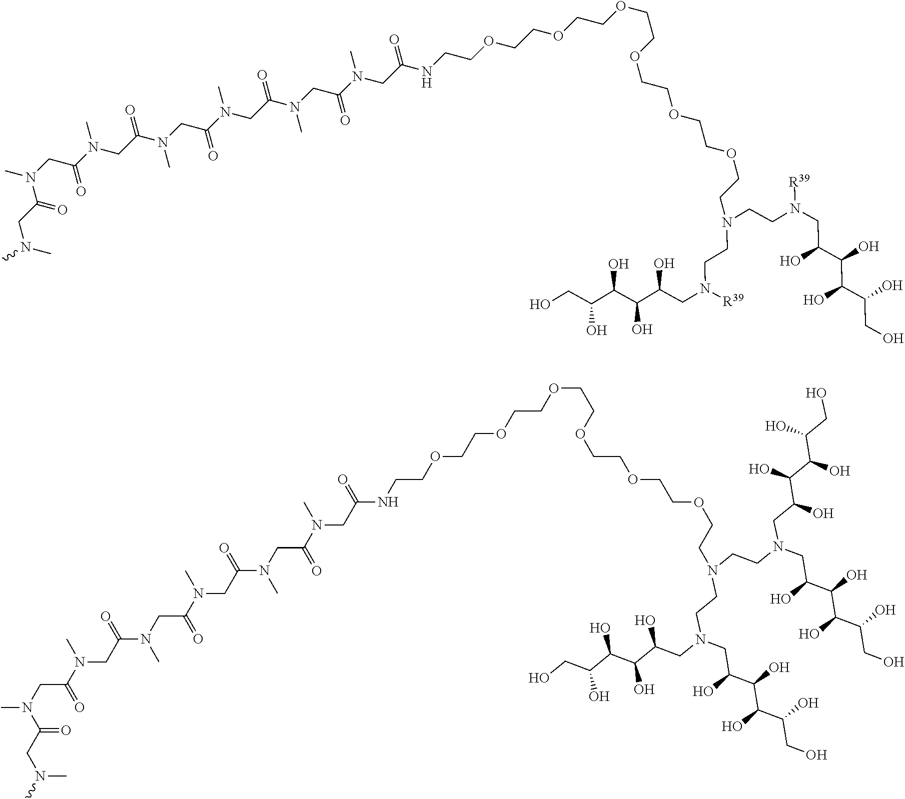

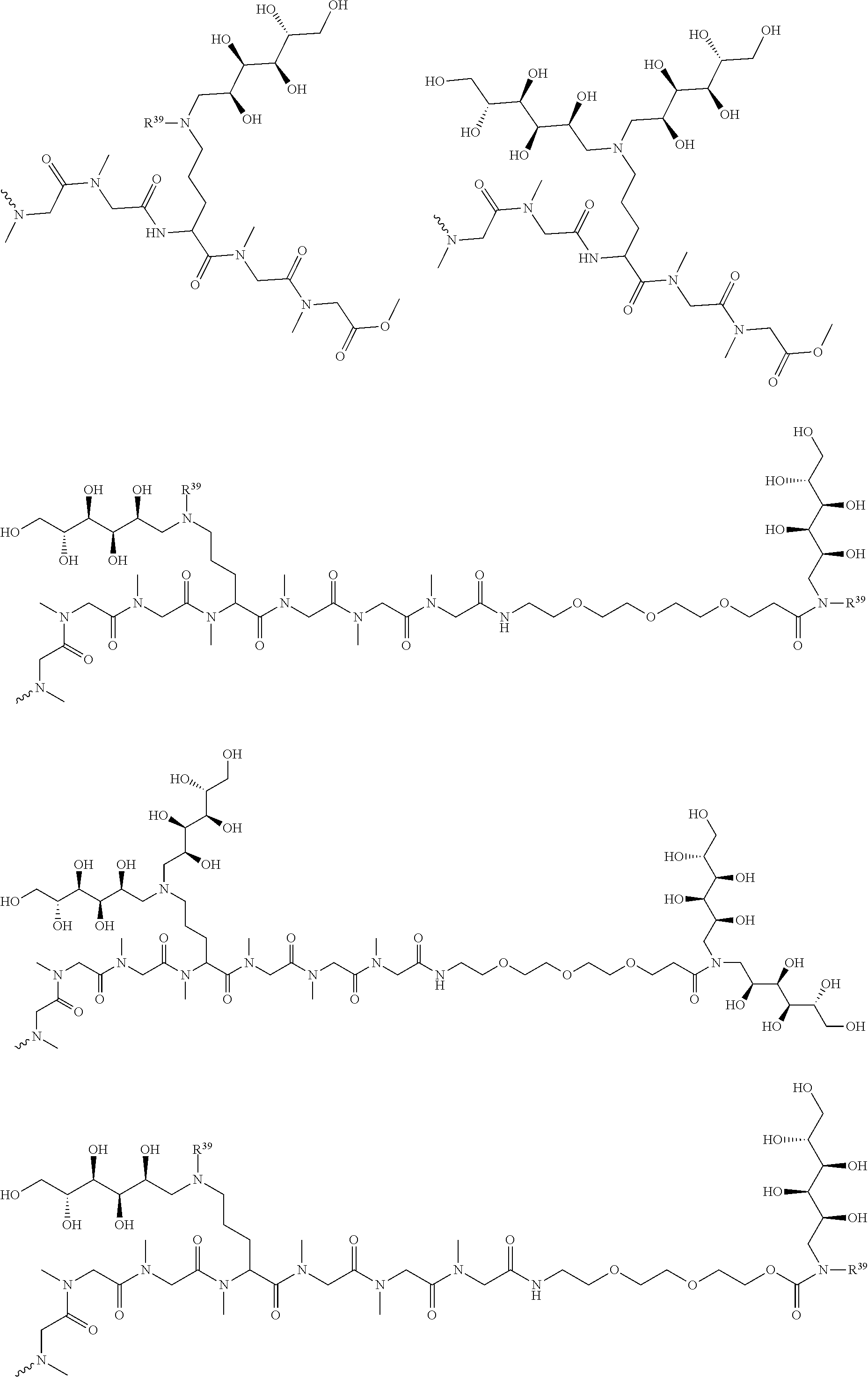

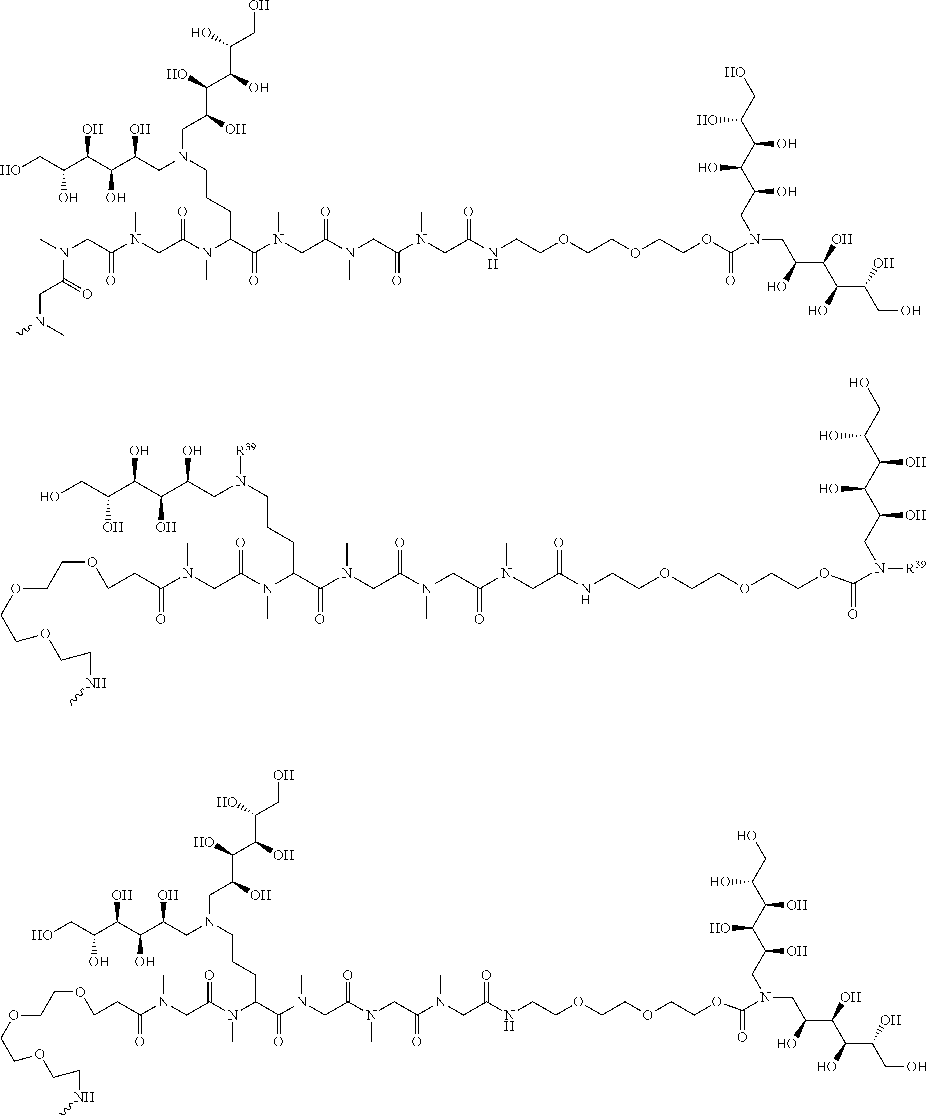

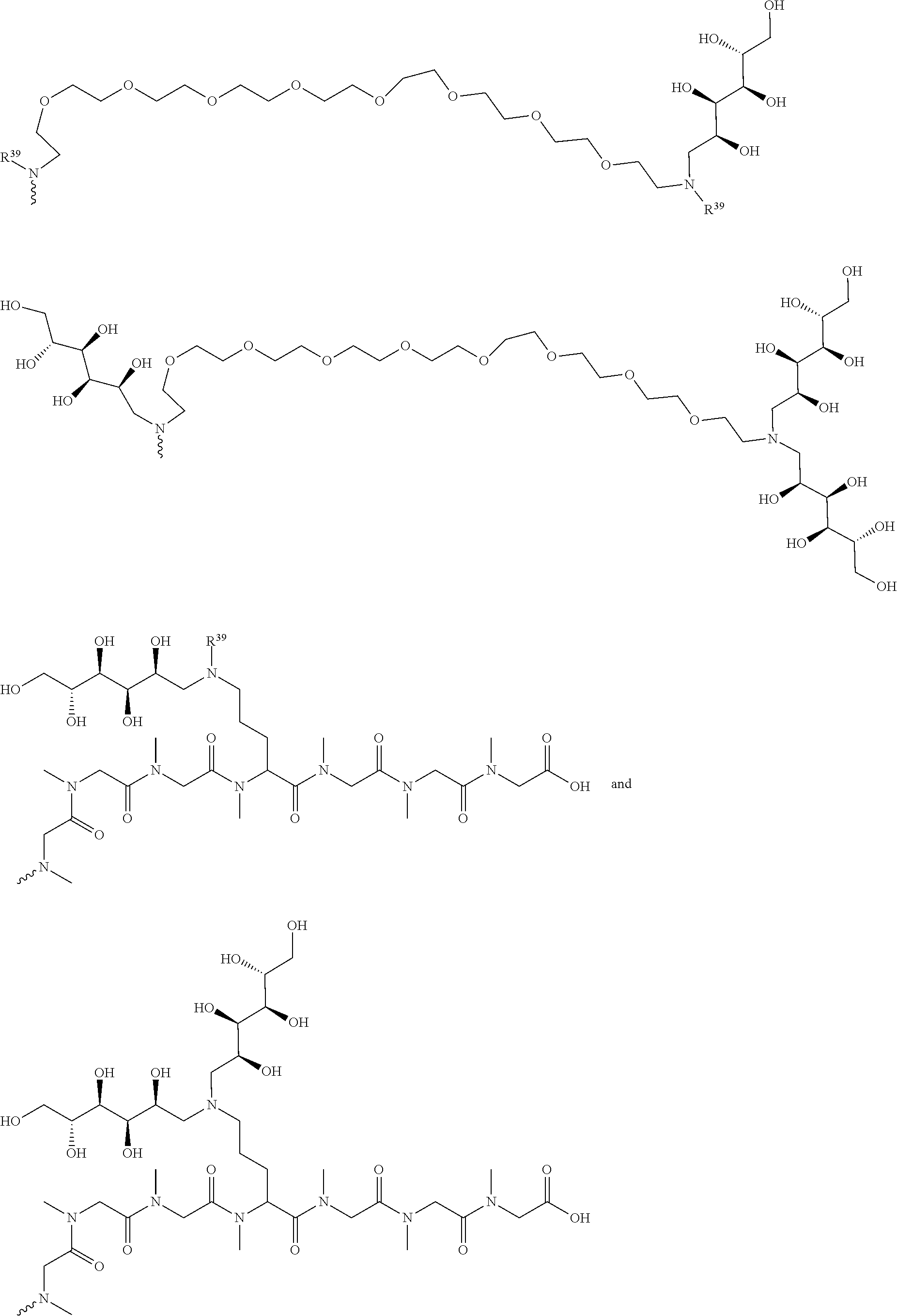

wherein each R is independently H or alkyl; each R³⁹ is independently selected from H, a linear monosaccharide and polyethylene glycol, optionally having from 1 to 24 ethylene glycol subunits; each n independently is 1-12; and the wavy line is an attachment site to R^{b}, or to the enzyme-cleavable group.

In some embodiments, one of R²⁴ and R²⁵ as described above is a linear monosaccharide and the other is a cyclic monosaccharide.

In some embodiments, provided are Linker compounds comprising a Polar group selected from the following, or a stereoisomer or salt thereof:

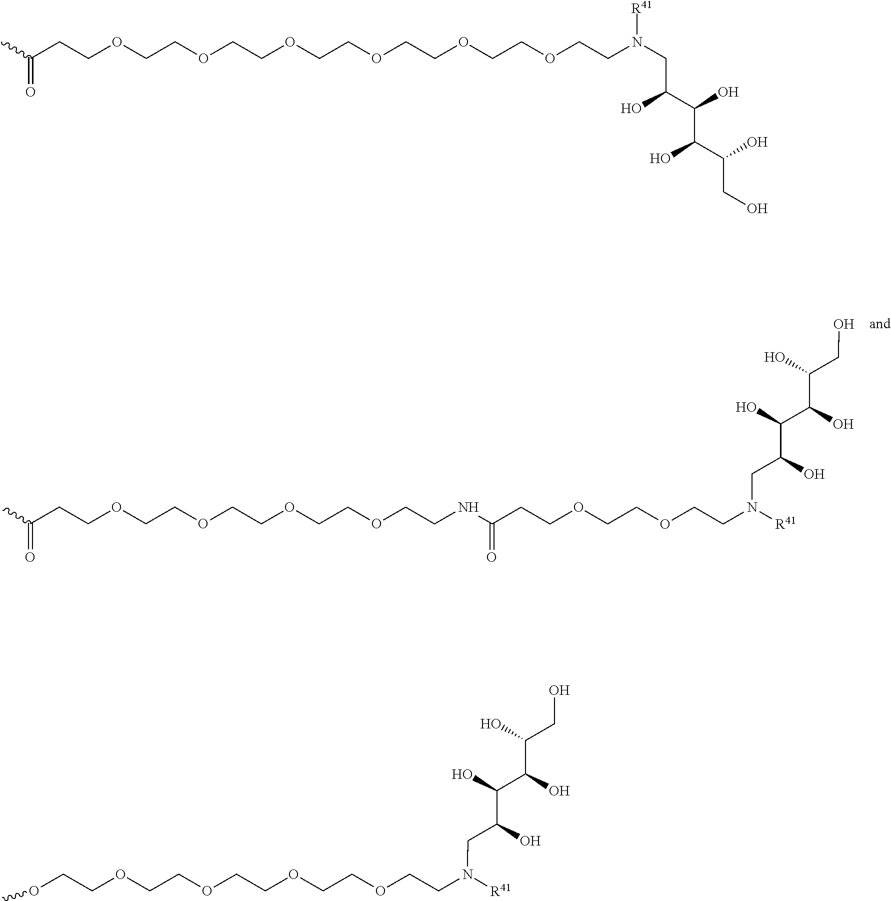

wherein R⁴¹ is a cyclic monosaccharide; and the wavy line is an attachment site to R^{b}, or to the enzyme-cleavable group.

In some embodiments, R²⁴ and R²⁵ as described above are independently a polyhydroxyl selected from a cyclic monosaccharide, disaccharide and polysaccharide.

In some embodiments, provided are Linker compounds comprising a Polar group selected from the following, or a stereoisomer or salt thereof:

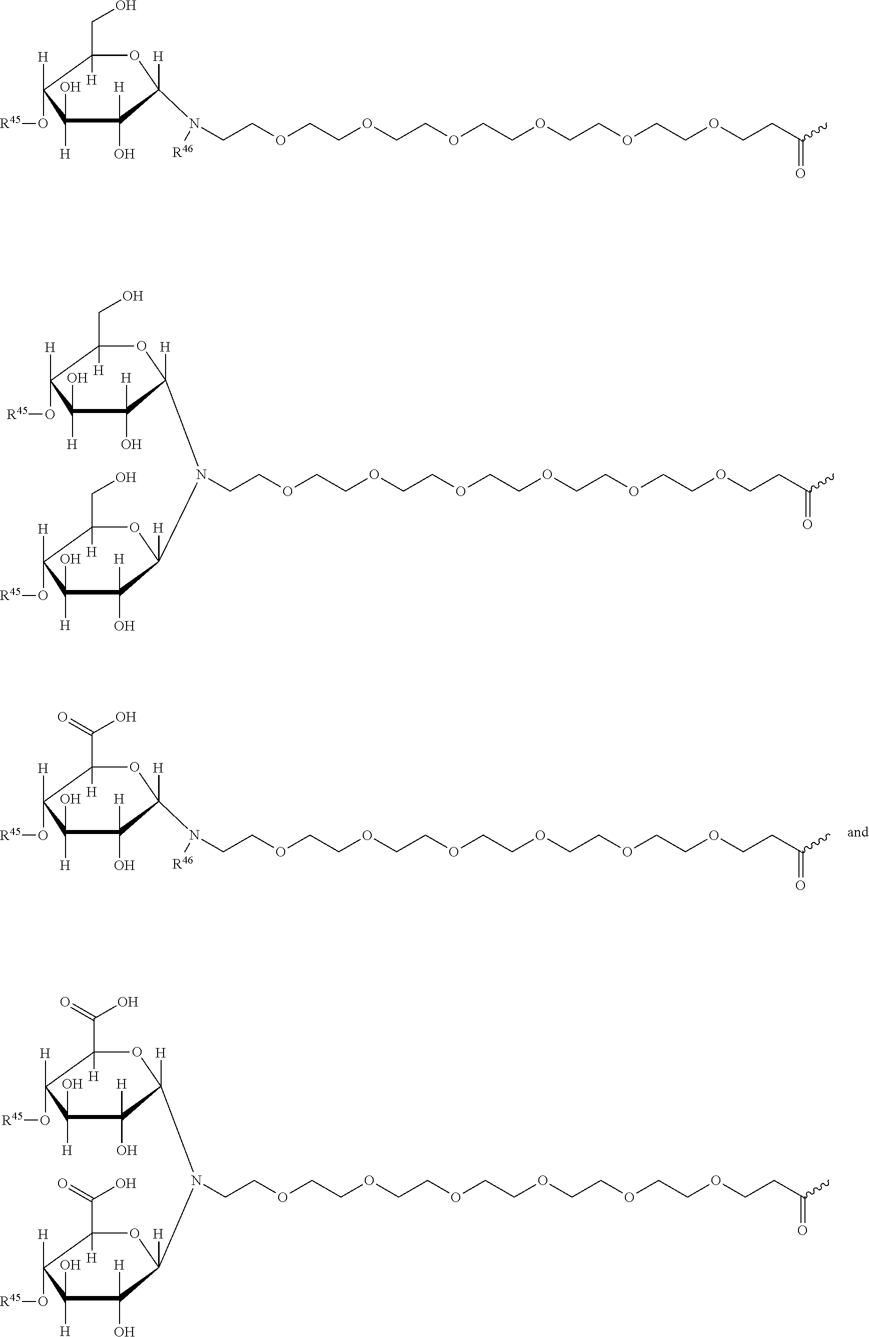

wherein each R⁴⁵ is selected from H and a monosaccharide, a disaccharide, or a polysaccharide; and R⁴⁶ is selected from a cyclic monosaccharide, disaccharide, or polysaccharide; and the wavy line is an attachment site to R^{b}, or to the enzyme-cleavable group.

In some embodiments, R²⁴ and R²⁵ as described above are independently selected from a linear monosaccharide and a substituted linear monosaccharide, wherein the substituted linear monosaccharide is substituted with a monosaccharide, a disaccharide or a polysaccharide.

In some embodiments, provided herein are Linker compounds comprising a Polar group selected from the following, or a stereoisomer or salt thereof:

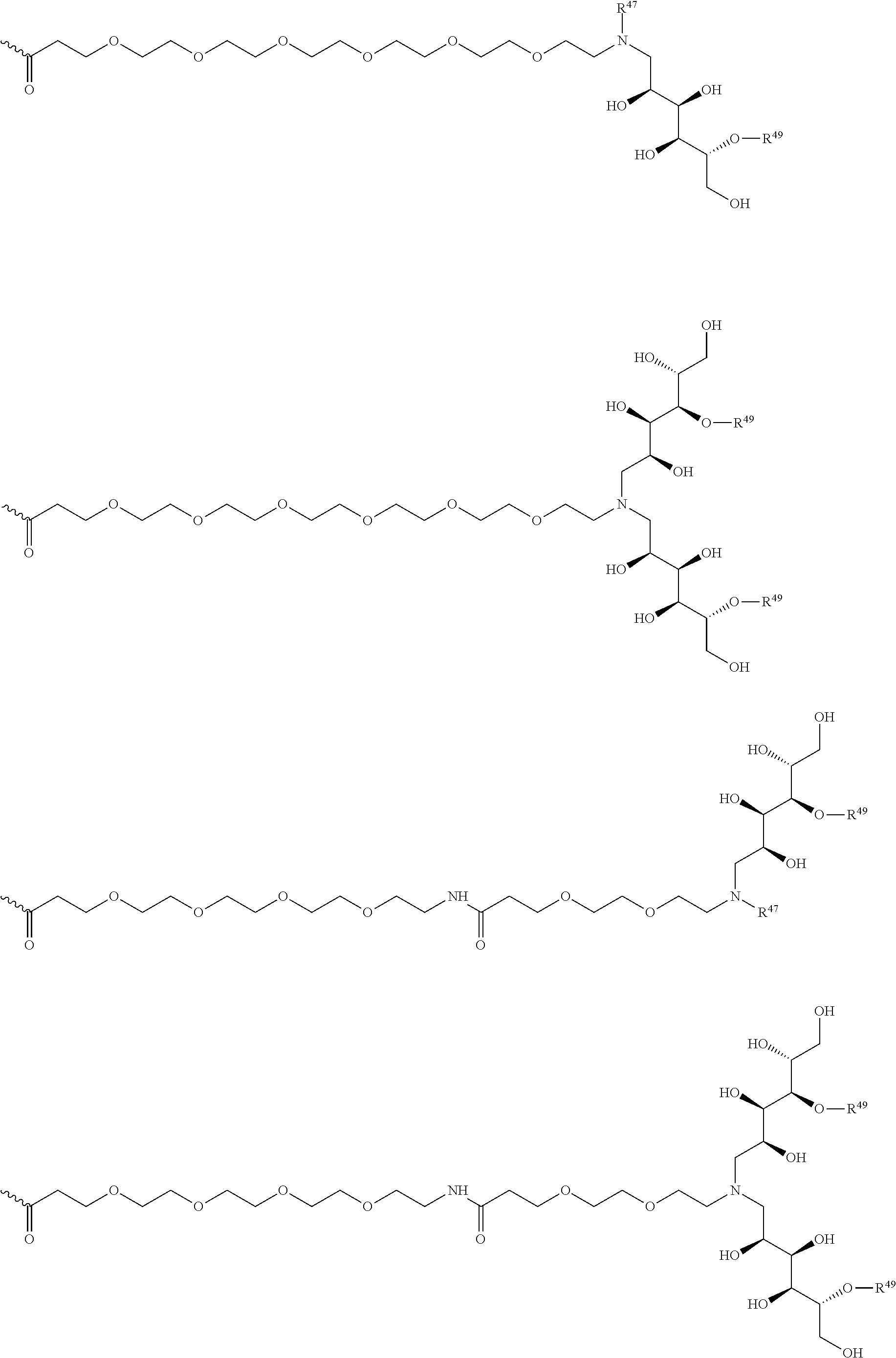

wherein R⁴⁷ is a linear monosaccharide; and each R⁴⁹ is selected from a monosaccharide, a disaccharide and a polysaccharide; and the wavy line is an attachment site to R^{b}, or to the enzyme-cleavable group.

In some embodiments, R²⁴ and R²⁵ as described above are independently selected from a linear monosaccharide and a substituted monosaccharide, wherein the substituted linear monosaccharide is substituted with one or more substituents selected from alkyl, O-alkyl, aryl, O-aryl, carboxyl, ester, or amide, and optionally further substituted with a monosaccharide, disaccharide or a polysaccharide.

In some embodiments, R²⁴ and R²⁵ as described above are independently selected from a linear monosaccharide and a substituted monosaccharide, wherein the substituted linear monosaccharide is substituted with one or more substituents selected from carboxyl, ester, and amide, and optionally further substituted with a monosaccharide, disaccharide, or a polysaccharide.

In some embodiments, provided are Linker compounds comprising a Polar group selected from the following, or a stereoisomer or salt thereof:

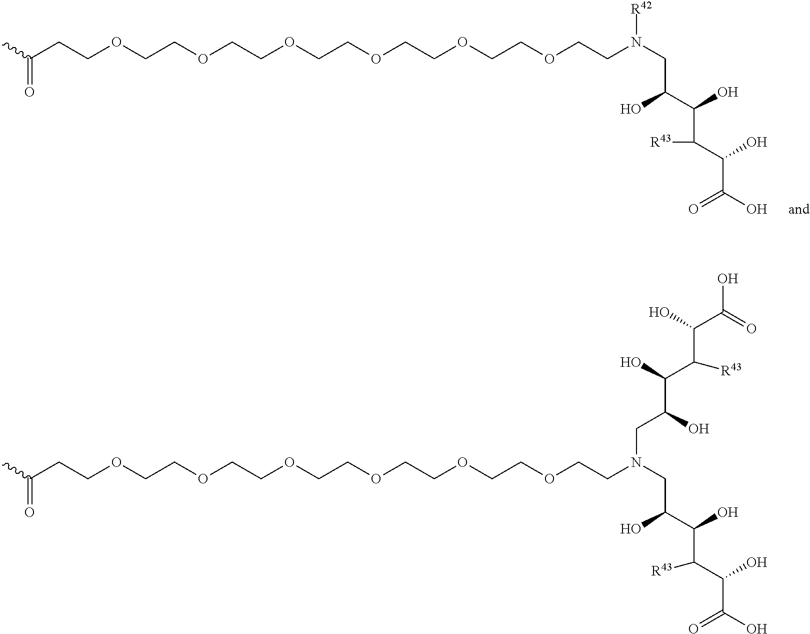

wherein each R⁴² is independently selected from a linear monosaccharide and a substituted linear monosaccharide; each R⁴³ is independently selected from alkyl, O-alkyl, aryl, O-aryl, carboxyl, ester, and amide; and the wavy line is an attachment site to R^{b}, or to the enzyme-cleavable group.

In some embodiments, provided are Linker compounds comprising a Polar group selected from the following, or a stereoisomer or salt thereof:

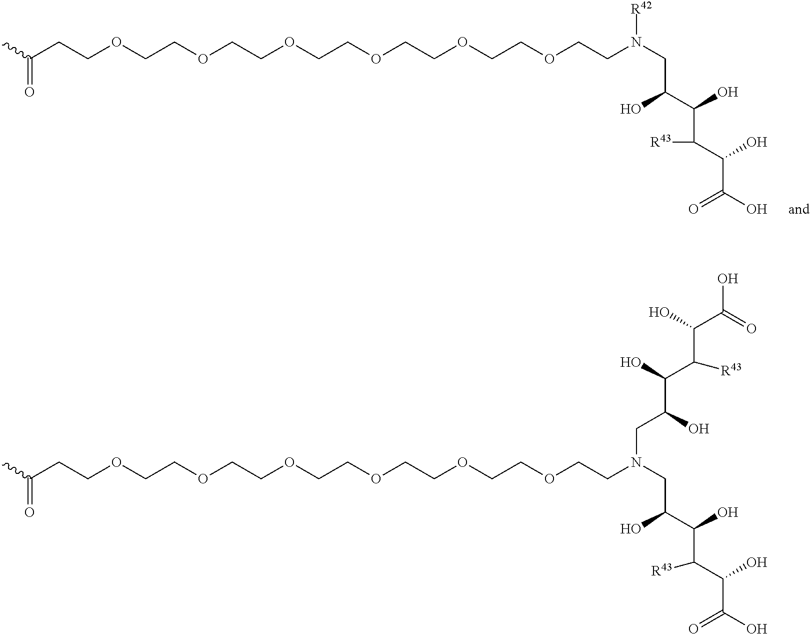

wherein each R⁴² is independently selected from a linear monosaccharide and a substituted linear monosaccharide; each R⁴³ is independently selected from hydroxyl, carboxyl, ester, and amide; and the wavy line is an attachment site to R^{b}, or to the enzyme-cleavable group.

In some embodiments, one of R²⁴ and R²⁵ as described above is a —C(O)-polyhydroxyl group or substituted —C(O)-polyhydroxyl group, and the other of R²⁴ and R²⁵ is a H, —C(O)-polyhydroxyl group, substituted —C(O)-polyhydroxyl group, polyhydroxyl group or substituted polyhydroxyl group; wherein the substituted —C(O)-polyhydroxyl group and polyhydroxyl group are substituted with a monosaccharide, a disaccharide, a polysaccharide, alkyl, —O-alkyl, aryl, carboxyl, ester, or amide.

In some embodiments, one of R²⁴ and R²⁵ as described above is a —C(O)-polyhydroxyl group or substituted —C(O)-polyhydroxyl group, and the other of R²⁴ and R²⁵ is a H, —C(O)-polyhydroxyl group, substituted —C(O)-polyhydroxyl group, polyhydroxyl group or substituted polyhydroxyl group; wherein the substituted —C(O)-polyhydroxyl group and polyhydroxyl group are substituted with a monosaccharide, a disaccharide, a polysaccharide, carboxyl, ester, or amide.

In some embodiments, provided are Linker compounds comprising a Polar group selected from the following, or a stereoisomer or salt thereof:

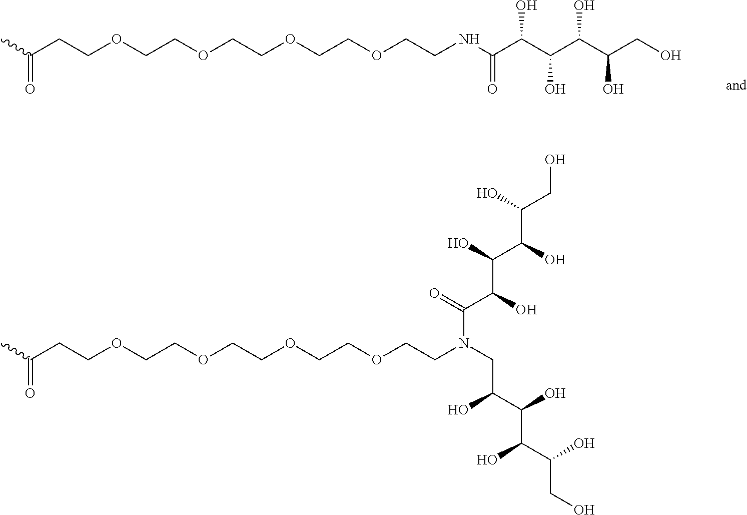

wherein the wavy line is an attachment site to R^{b}, or to the enzyme-cleavable group.

In some embodiments, R²⁴ and R²⁵ as described above are independently selected from a H, substituted —C₁-C₈ alkyl, substituted —C₁-C₄ alkyl or substituted —C₁-C₃ alkyl; provided that both R²⁴ and R²⁵ are not H; wherein substituted —C₁-C₈ alkyl, —C₁-C₄ alkyl, and —C₁-C₃ alkyl are substituted with hydroxyl and/or carboxyl.

In some embodiments, R²⁴ and R²⁵ as described above are independently H or substituted —C₁-C₈ alkyl; provided that both R²⁴ and R²⁵ are not H; wherein substituted —C₁-C₈ alkyl is substituted with hydroxyl and/or carboxyl.

In some embodiments, provided are Linker compounds comprising a Polar group selected from the following, or a stereoisomer or salt thereof:

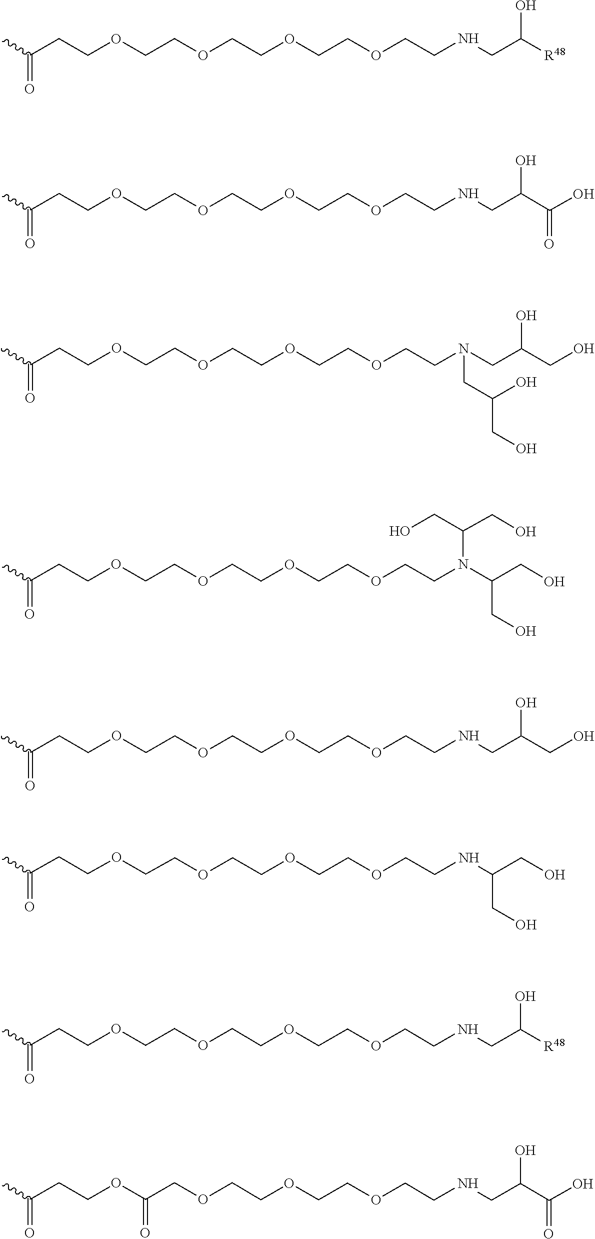

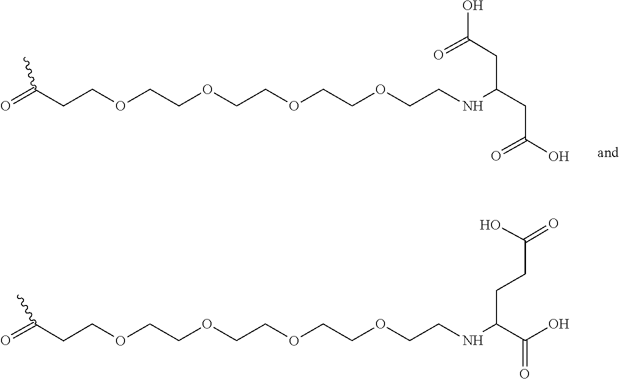

wherein R⁴⁸ is selected from H, OH, CH₂OH, COOH or —C₁-C₆ alkyl substituted with hydroxyl or carboxyl; and the wavy line is an attachment site to R^{b}, or to the enzyme-cleavable group.

In some embodiments, one of R²⁴ and R²⁵ as described above is selected from H, substituted —C(O)—C₁-C₈ alkyl, substituted —C(O)—C₁-C₄ alkyl, and substituted —C(O)—C₁-C₃ alkyl and the other of R²⁴ and R²⁵ is selected from substituted —C(O)—C₁-C₈ alkyl, substituted —C(O)—C₁-C₄ alkyl, substituted —C(O)—C₁-C₃ alkyl, substituted —C₁-C₈ alkyl, substituted —C₁-C₄ alkyl, and substituted —C₁-C₃ alkyl, wherein substituted —C(O)—C₁-C₈ alkyl, substituted —C(O)—C₁-C₄ alkyl, substituted —C(O)—C₁-C₃ alkyl, substituted —C₁-C₈ alkyl, —C₁-C₄ alkyl and —C₁-C₃ alkyl are substituted with hydroxyl and/or carboxyl.

In some embodiments, wherein one of R²⁴ and R²⁵ is H or substituted —C(O)—C₁-C₈ alkyl, and the other of R²⁴ and R²⁵ is substituted —C(O)—C₁-C₈ alkyl, or substituted —C₁-C₈ alkyl, wherein substituted —C(O)—C₁-C₈ alkyl and substituted —C₁-C₈ alkyl, are substituted with hydroxyl and/or carboxyl.

In some embodiments, provided are Linker compounds comprising a Polar group selected from the following, or a stereoisomer or salt thereof:

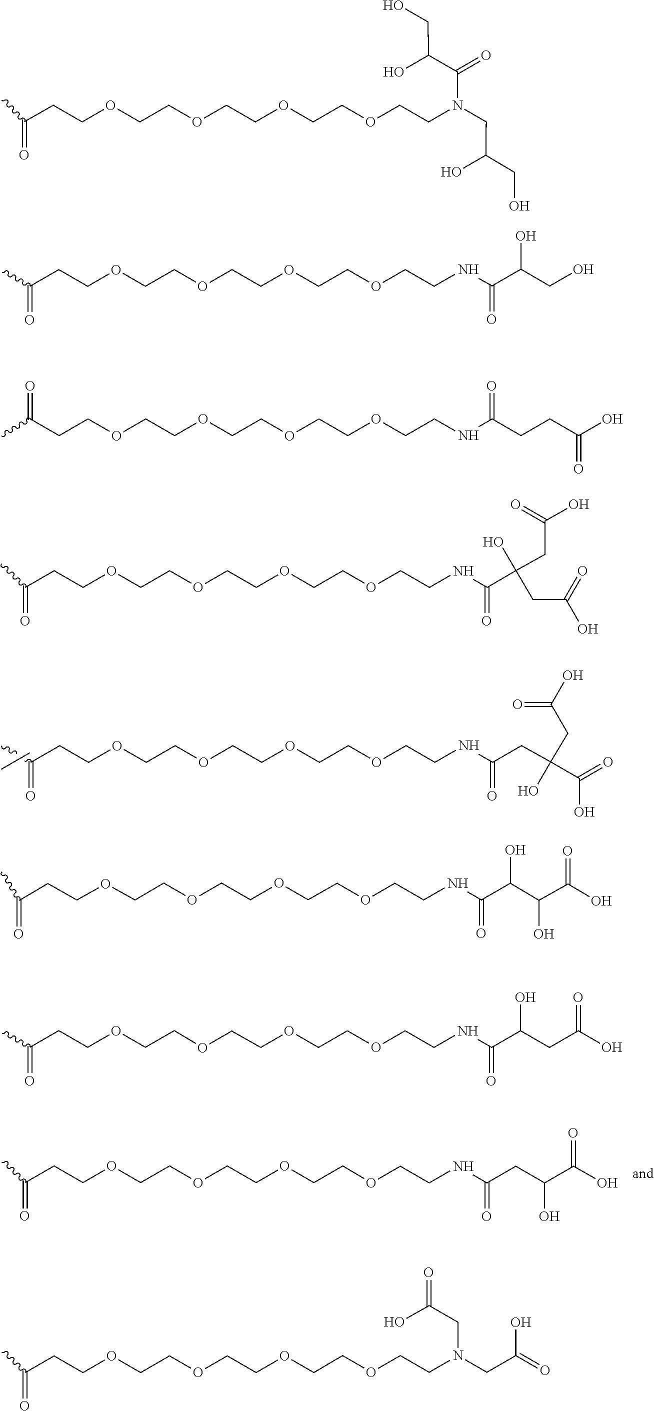

wherein the wavy line is an attachment site to R^{b}, or to the enzyme-cleavable group.

In some embodiments, R²⁴ and R²⁵ as described above are selected from H and optionally substituted aryl; provided that both R²⁴ and R²⁵ are not H.

In some embodiments, provided as Linker compounds comprising a Polar group selected from the following, or a stereoisomer or salt thereof:

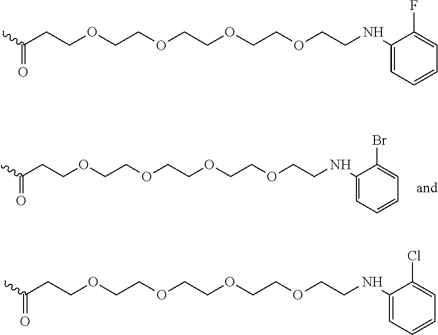

wherein the wavy line is an attachment site to R^{b}, or to the enzyme-cleavable group.

In some embodiments, R²⁴ and R²⁵ as described above together form an optionally substituted C₃-C₈ heterocycle or heteroaryl.

In some embodiments, provided are Linker compounds comprising a Polar group having the following structure:

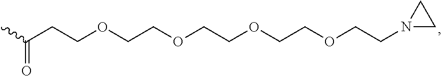

or a stereoisomer or salt thereof, wherein the wavy line is an attachment site to R^{b}, or to the enzyme-cleavable group.

In some embodiments, R²⁴ and R²⁵ as described above are independently selected from H and a chelator, wherein the chelator is optionally attached to the nitrogen of —NR²⁴R²⁵ by an alkylene, arylene, carbocyclo, heteroarylene or heterocarbocylo; provided that both R²⁴ and R²⁵ are not H. For example, in some embodiments the chelator is selected from ethylenediaminetetraacetic acid (EDTA), diethylenetriaminepentaacetic acid (DTPA), triethylenetetraminehexaacetic acid (TTHA), benzyl-DTPA, 1,4,7,10-tetraazacyclododeca′e-″N′″″,N′″-tetraacetic acid (DOTA), benzyl-DOTA, 1,4,7-triazacyclonona′e-″N′,N″-triacetic acid (NOTA), benzyl-NOTA, 1,4,8,11-tetraazacyclotetradecane-1,4,8,11-tetraacetic acid (TETA) a′d N,N′-dialkyl substituted piperazine.

In some embodiments, provided are Linker compounds comprising a Polar group selected from the following, or a stereoisomer or salt thereof:

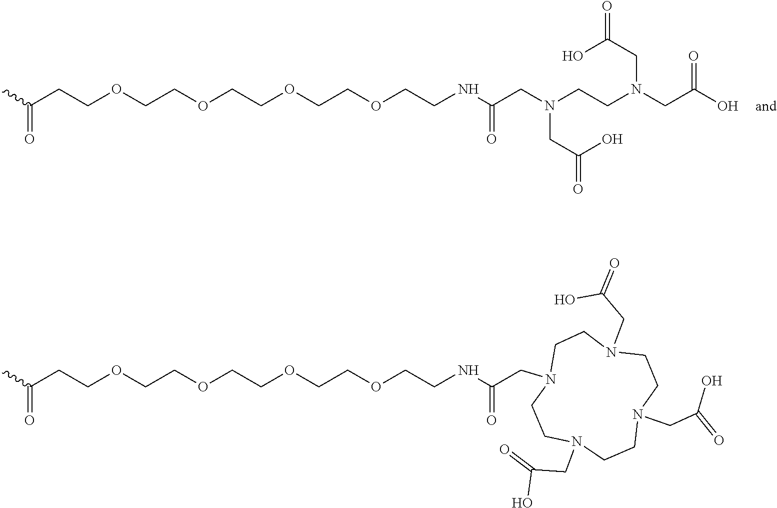

wherein the wavy line is an attachment site to R^{b}, or to the enzyme-cleavable group.

In some embodiments, each monosaccharide as described above is independently selected from: a C5 or C6 sugar selected from glucose, ribose, galactose, mannose, arabinose, 2-deoxyglucose, glyceraldehyde, erythrose, threose, xylose, lyxose, allose, altrose, gulose, idose talose, aldose, ketose, glucosamine, N-acetyl glucosamine, galactosamine, and N-acetyl galactosamine; a sugar acid selected from gluconic acid, aldonic acid, uronic acid and ulosonic acid; or an amino sugar is selected from glucosamine, N-acetyl glucosamine, galactosamine, and N-acetyl galactosamine.

In some embodiments, each monosaccharide as described above is independently selected from: a C5 or C6 sugar selected from glucose, ribose, galactose, mannose, arabinose, 2-deoxyglucose, glyceraldehyde, erythrose, threose, xylose, lyxose, allose, altrose, gulose, idose talose, aldose, and ketose, a sugar acid selected from gluconic acid, aldonic acid, uronic acid and ulosonic acid; or an amino sugar selected from glucosamine, N-acetyl glucosamine, galactosamine, and N-acetyl galactosamine.

In some embodiments, the attachment site as described above is formed from a functional group of a precursor compound of the Polar group, said functional group selected from halo, aldehyde, carboxyl, amino, alkynyl, azido, hydroxyl, carbonyl, carbamate, thiol, urea, thiocarbamate, thiourea, sulfonamide, acyl sulfonamide, alkyl sulfonate, triazole, azadibenzocyclooctyne, hydrazine, carbonylalkylheteroaryl, and protected forms thereof.

In some embodiments, provided are Linker compounds comprising a Polar group having a formula selected from the following:

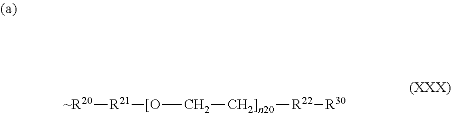

or a stereoisomer or salt thereof, wherein: R²⁰ is an attachment group to site R^{b}, or to the enzyme-cleavable group; R²¹ and R²² are each independently, a bond or C₁-C₃ alkylene groups; R³⁰ is selected from an optionally substituted C₃-C₁₀ carbocycle; thiourea; optionally substituted thiourea; urea; optionally substituted urea; sulfamide; alkyl sulfamide; acyl sulfamide, optionally substituted alkyl sulfamide; optionally substituted acyl sulfamide; sulfonamide; optionally substituted sulfonamide; guanidine, including alkyl and aryl guanidine; phosphoramide; or optionally substituted phosphoramide; or R³⁰ is selected from azido, alkynyl, substituted alkynyl, —NH—C(O)-alkynyl, —NH—C(O)-alkynyl-R⁶⁵; cyclooctyne; —NH-cyclooctyne, —NH—C(O)-cyclooctyne, or —NH-(cyclooctyne)₂; wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and n20 is 2 to 26;

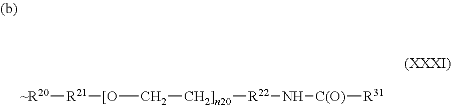

or a stereoisomer or salt thereof, wherein: R²⁰ is an attachment group to site R^{b}, or to the enzyme-cleavable group; R²¹ and R²² are each, independently, a bond or C₁-C₃ alkylene groups; R³¹ is a branched polyethylene glycol chain, each branch having 1 to 26 ethylene glycol subunits and each branch having an R³⁵ at its terminus; R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and n20 is 2 to 26;

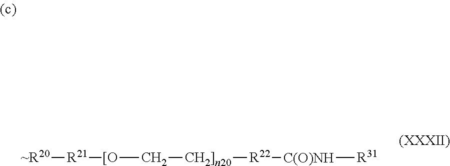

or a stereoisomer or salt thereof, wherein: R²⁰ is an attachment group to site R^{b}, or to the enzyme-cleavable group; R²¹ and R²² are each, independently, a bond or C₁-C₃ alkylene groups; R³¹ is a branched polyethylene glycol chain, each branch, independently, having 1 to 26 ethylene glycol subunits and each branch having an R³⁵ at its terminus; R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle and optionally substituted heteroaryl; and n20 is 2 to 26;

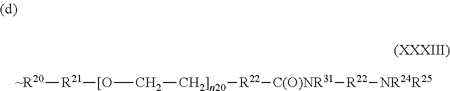

or a stereoisomer or salt thereof, wherein: R²⁰ is an attachment group to site R^{b}, or to the enzyme-cleavable group; R³¹ is H or R²²—NR²⁴R²⁵; R²¹ and R²² are each, independently, a bond or C₁-C₃ alkylene groups; R²⁴ and R²⁵ are each independently selected from a H; polyhydroxyl group; substituted polyhydroxyl group; —C(O)-polyhydroxyl group; substituted —C(O)-polyhydroxyl group, provided that R²⁴ and R²⁵ are not both H; and n20 is 2 to 26;

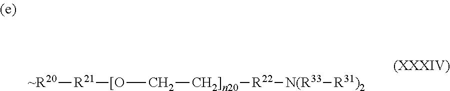

or a stereoisomer or salt thereof, wherein: R²⁰ is an attachment group to site R^{b}, or to the enzyme-cleavable group; R²¹ and R²² are each, independently, a bond or C₁-C₃ alkylene groups; R³¹ is a branched polyethylene glycol chain, each branch having 1 to 26 ethylene glycol subunits and each branch having an R³⁵ at its terminus; R³³ is C₁-C₃ alkylene, C₁-C₃ alkylene-C(O), —C(O)—C₁-C₃ alkylene, or —C(O)—C₁-C₃ alkylene-C(O); R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and n20 is 2 to 26;

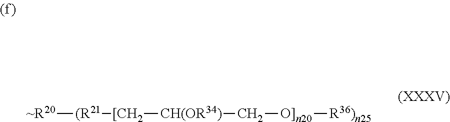

or a stereoisomer or salt thereof, wherein: R²⁰ is an attachment group to site R^{b}, or to the enzyme-cleavable group; each R²¹ is independently a bond, —O— or C₁-C₃ alkylene group; each R³⁴ is independently H, —[CH₂—CH(OH)—CH₂—O]ₙ₂₀—R³⁶, —C(O)—NR²⁴R²⁵ or —C(O)N(R^{N})—C₁-C₆alkylene-NR²⁴R²⁵; R^{N} is H or C₁-C₄alkyl; R²⁴ and R²⁵ are each independently selected from a H; polyhydroxyl group; or substituted polyhydroxyl group, provided that both R²⁴ and R²⁵ are not H; each R³⁶ is independently H, C₁-C₆alkylene-C(OH)H—NR⁴⁴R⁴⁵, C₁-C₆alkylene-C(OH)H—C₁-C₆alkylene-NR⁴⁴R⁴⁵, —C(O)—NR²⁴R²⁵, —C(O)N(R^{N})—C₁-C₆alkylene-NR²⁴R²⁵, C₁-C₆alkylene-C(O)NR²⁴R²⁵ or C₁-C₆alkylene-CO₂R³⁷; each R³⁷ is independently H or C₁-C₆ alkyl; R⁴⁴ and R⁴⁵ are each independently selected from a H; polyhydroxyl group; substituted polyhydroxyl group; —C(O)-polyhydroxyl group; and substituted —C(O)-polyhydroxyl group, provided that both R⁴⁴ and R⁴⁵ are not H; each n20 is independently 1 to 26; and n25 is 2 or 2;

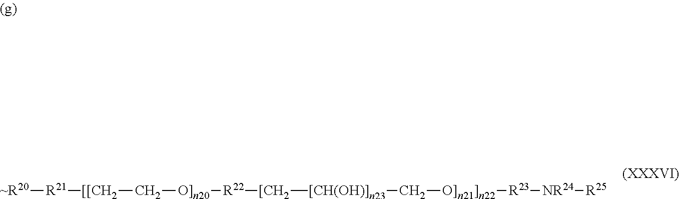

or a stereoisomer or salt thereof, wherein: R²⁰ is an attachment group to site R^{b}, or to the enzyme-cleavable group; R²¹, R²² and R²³ are each independently a bond or C₁-C₃ alkylene group; R²⁴ and R²⁵ are each independently selected from a H; polyhydroxyl group; substituted polyhydroxyl group; —C(O)-polyhydroxyl group; and substituted —C(O)-polyhydroxyl group, provided that R²⁴ and R²⁵ are not both H; each n20 is independently 0 to 26, and each n21 is independently 0 to 26, with the proviso that at least one of n20 or n21 is 2 to 26; n22 is 1 to 5; each n23 is independently 1 or 2;

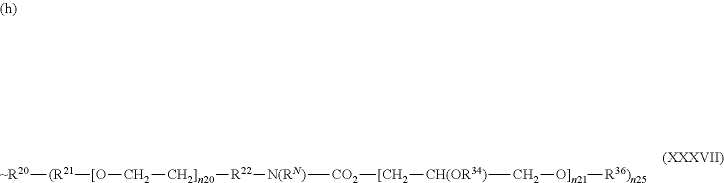

(i) or a stereoisomer or salt thereof, wherein: R²⁰ is an attachment group to site R^{b}, or to the enzyme-cleavable group; R²¹ and R²² are each independently a bond or C₁-C₃ alkylene groups; R^{N} is H or C₁-C₄alkyl; R²⁴ and R²⁵ are each independently selected from a H; polyhydroxyl group; and substituted polyhydroxyl group, provided that both R⁴⁴ and R⁴⁵ and not H; each R³⁴ is independently H, —[CH₂—CH(OH)—CH₂—O]ₙ₂₀—R³⁶ or —C(O)N(R^{N})—C₁-C₆alkylene-NR²⁴R²⁵; each R³⁶ is independently H, C₁-C₆alkylene-C(OH)H—NR⁴⁴R⁴⁵, C₁-C₆ alkylene-C(OH)H—C₁-C₆alkylene-NR⁴⁴R⁴⁵, —C(O)N(R^{N})—C₁-C₆alkylene-NR²⁴R²⁵, C₁-C₆alkylene-C(O)NR²⁴R²⁵ or C₁-C₆alkylene-CO₂R³⁷; each R³⁷ is independently H or C₁-C₆ alkyl; R⁴⁴ and R⁴⁵ are each independently selected from a H; polyhydroxyl group; substituted polyhydroxyl group; —C(O)-polyhydroxyl group; and substituted —C(O)-polyhydroxyl group, provided that both R⁴⁴ and R⁴⁵ are not H; n20 is 2 to 26; n21 is 1 to 26; and n25 is 1 or

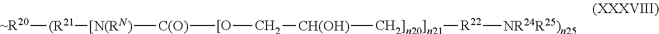

or a stereoisomer or salt thereof, wherein: R²⁰ is an attachment group to site R^{b}, or to the enzyme-cleavable group; R²¹ and R²² are each independently a bond or C₁-C₃ alkylene groups; R^{N} is H or C₁-C₄alkyl; R²⁴ and R²⁵ are each independently selected from a H; polyhydroxyl group; and substituted polyhydroxyl group, provided that R²⁴ and R²⁵ are not both H; n20 is 2 to 26; n21 is 1 to 4; and n25 is 1, 2 or 3;

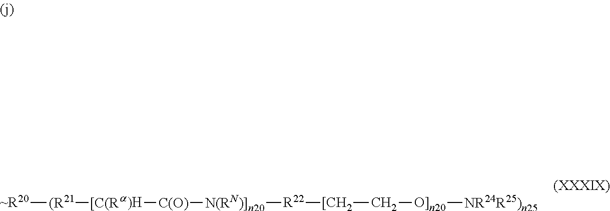

or a stereoisomer or salt thereof, wherein: R²⁰ is an attachment group to site R^{b}, or to the enzyme-cleavable group; R²¹ and R²² are each, independently, a bond, C₁-C₃ alkylene, —C₁-C₃alkylene-[O—CH₂—CH₂-]ₙ₂₀, —[CH₂—CH₂—O]ₙ₂₀—C₁-C₃alkylene- or —C₁-C₃alkylene-[O—CH₂—CH₂-]ₙ₂₀-C(O)—; each R^{α} is independently H or —R²²—NR²⁴R²⁵; each R^{N} is independently H, C₁-C₆ alkyl or —R²²—NR²⁴R²⁵; R²⁴ and R²⁵ are each independently selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; and —C(O)—R²⁸, where R²⁸ is a Sugar unit of formula (XII) or (XIII); or —NR²⁴R²⁵ together from a C₃-C₈ heterocycle, provided that R²⁴ and R²⁵ are not both H; each n20 is independently 0 in 26, with the proviso that at least one n20 is 2 to 26; and n25 is 1 nr 2; and

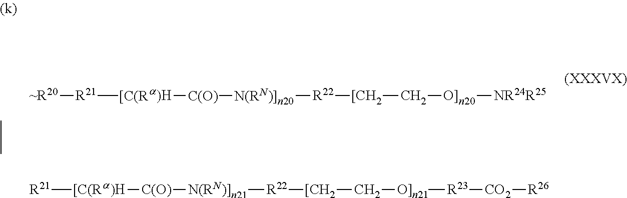

or a stereoisomer or salt thereof, wherein: R²⁰ is an attachment group to site R^{b}, or to the enzyme-cleavable group; R²¹, R²² and R²³ are each, independently, a bond, C₁-C₃ alkylene, —C₁-C₃alkylene-[O—CH₂—CH₂-]ₙ₂₀, —[CH₂—CH₂—O]ₙ₂₀—C₁-C₃alkylene- or —C₁-C₃alkylene-[O—CH₂—CH₂-]ₙ₂₀-C(O)—; each R^{α} is independently H or —R²²—NR²⁴R²⁵; each R^{N} is independently H, C₁-C₆ alkyl or —R²²—NR²⁴R²⁵; R²⁴ and R²⁵ are each independently selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; and —C(O)—R²⁸, where R²⁸ is a Sugar unit of formula (XII) or (XIII); or —NR²⁴R²⁵ together from a C₃-C₈ heterocycle, provided that R²⁴ and R²⁵ are not both H; R²⁶ is H or C₁-C₆ alkyl; each n20 is independently 0 to 26, with the proviso that at least one n20 is 2 to 26; and each n21 is independently 0 to 26, with the proviso that at least one n21 is 2 to 26.

In some embodiments, provided are Linker compounds comprising a Polar group having a formula selected from the following:

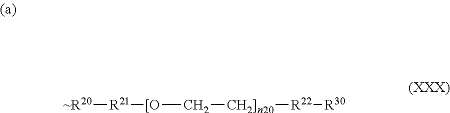

or a stereoisomer or salt thereof, wherein: R²⁰ is an attachment group to site R^{b}, or to the enzyme-cleavable group; R²¹ and R²² are each independently, a bond or C₁-C₃ alkylene groups; R³⁰ is selected from an optionally substituted C₃-C₁₀ carbocycle; thiourea; optionally substituted thiourea; urea; optionally substituted urea; sulfamide; alkyl sulfamide; acyl sulfamide, optionally substituted alkyl sulfamide; optionally substituted acyl sulfamide; sulfonamide; optionally substituted sulfonamide; guanidine, including alkyl and aryl guanidine; phosphoramide; or optionally substituted phosphoramide; or R³⁰ is selected from azido, alkynyl, substituted alkynyl, —NH—C(O)-alkynyl, —NH—C(O)-alkynyl-R⁶⁵; cyclooctyne; —NH-cyclooctyne, —NH—C(O)-cyclooctyne, or —NH-(cyclooctyne)₂; wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and n20 is 2 to 26;

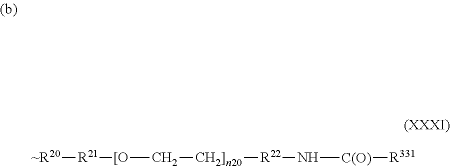

or a stereoisomer or salt thereof, wherein: R²⁰ is an attachment group to site R^{b}, or to the enzyme-cleavable group; R²¹ and R²² are each, independently, a bond or C₁-C₃ alkylene groups; R³¹ is a branched polyethylene glycol chain, each branch having 1 to 26 ethylene glycol subunits and each branch having an R³⁵ at its terminus; R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and n20 is 2 to 26;

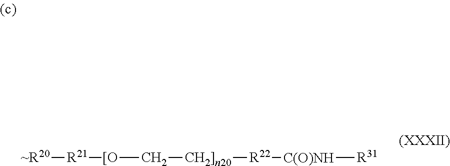

or a stereoisomer or salt thereof, wherein: R²⁰ is an attachment group to site R^{b}, or to the enzyme-cleavable group; R²¹ and R²² are each, independently, a bond or C₁-C₃ alkylene groups; R³¹ is a branched polyethylene glycol chain, each branch, independently, having 1 to 26 ethylene glycol subunits and each branch having an R³⁵ at its terminus; R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle and optionally substituted heteroaryl; and n20 is 2 to 26;

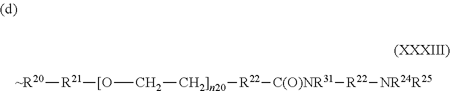

or a stereoisomer or salt thereof, wherein: R²⁰ is an attachment group to site R^{b}, or to the enzyme-cleavable group; R³¹ is H or R²²—NR²⁴R²⁵; R²¹ and R²² are each, independently, a bond or C₁-C₃ alkylene groups; R²⁴ and R²⁵ are each independently selected from a H; polyhydroxyl group; substituted polyhydroxyl group; —C(O)-polyhydroxyl group; substituted —C(O)-polyhydroxyl group, provided that R²⁴ and R²⁵ are not both H; and n20 is 2 to 26;

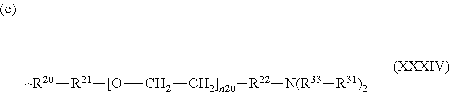

or a stereoisomer or salt thereof, wherein: R²⁰ is an attachment group to site R^{b}, or to the enzyme-cleavable group; R²¹ and R²² are each, independently, a bond or C₁-C₃ alkylene groups; R³¹ is a branched polyethylene glycol chain, each branch having 1 to 26 ethylene glycol subunits and each branch having an R³⁵ at its terminus; R³³ is C₁-C₃ alkylene, C₁-C₃ alkylene-C(O), —C(O)—C₁-C₃ alkylene, or —C(O)—C₁-C₃ alkylene-C(O); R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and n20 is 2 to 26;

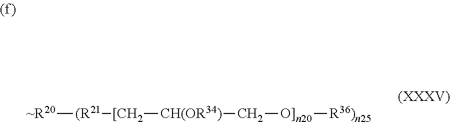

or a stereoisomer or salt thereof, wherein: R²⁰ is an attachment group to site R^{b}, or to the enzyme-cleavable group; each R²¹ is independently a bond, —O— or C₁-C₃ alkylene group; each R³⁴ is independently H, —[CH₂—CH(OH)—CH₂—O]ₙ₂₀—R³⁶, —C(O)—NR²⁴R²⁵ or —C(O)N(R^{N})—C₁-C₆alkylene-NR²⁴R²⁵; R^{N} is H or C₁-C₄alkyl; R²⁴ and R²⁵ are each independently selected from a H; polyhydroxyl group; or substituted polyhydroxyl group, provided that both R²⁴ and R²⁵ are not H; each R³⁶ is independently H, C₁-C₆alkylene-C(OH)H—NR⁴⁴R⁴⁵, C₁-C₆alkylene-C(OH)H—C₁-C₆alkylene-NR⁴⁴R⁴⁵, —C(O)—NR²⁴R²⁵, —C(O)N(R^{N})—C₁-C₆alkylene-NR²⁴R²⁵, C₁-C₆alkylene-C(O)NR²⁴R²⁵ or C₁-C₆alkylene-CO₂R³⁷; each R³⁷ is independently H or C₁-C₆ alkyl; R⁴⁴ and R⁴⁵ are each independently selected from a H; polyhydroxyl group; substituted polyhydroxyl group; —C(O)-polyhydroxyl group; and substituted —C(O)-polyhydroxyl group, provided that both R⁴⁴ and R⁴⁵ are not H; each n20 is independently 1 to 26; and n25 is 2 or 2;

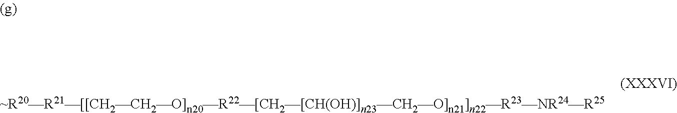

or a stereoisomer or salt thereof, wherein: R²⁰ is an attachment group to site R^{b}, or to the enzyme-cleavable group; R²¹, R²² and R²³ are each independently a bond or C₁-C₃ alkylene group; R²⁴ and R²⁵ are each independently selected from a H; polyhydroxyl group; substituted polyhydroxyl group; —C(O)-polyhydroxyl group; and substituted —C(O)-polyhydroxyl group, provided that R²⁴ and R²⁵ are not both H; each n20 is independently 0 to 26, and each n21 is independently 0 to 26, with the proviso that at least one of n20 or n21 is 2 to 26; n22 is 1 to 5; each n23 is independently-1 or 2;

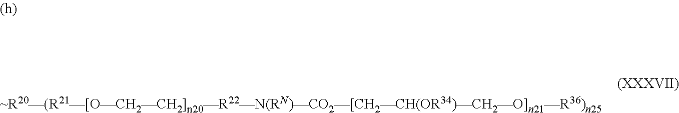

(i) or a stereoisomer or salt thereof, wherein: R²⁰ is an attachment group to site R^{b}, or to the enzyme-cleavable group; R²¹ and R²² are each independently a bond or C₁-C₃ alkylene groups; R^{N} is H or C₁-C₄alkyl; R²⁴ and R²⁵ are each independently selected from a H; polyhydroxyl group; and substituted polyhydroxyl group, provided that both R⁴⁴ and R⁴⁵ and not H; each R³⁴ is independently H, —[CH₂—CH(OH)—CH₂—O]ₙ₂₀—R³⁶ or —C(O)N(R^{N})—C₁-C₆alkylene-NR²⁴R²⁵; each R³⁶ is independently H, C₁-C₆alkylene-C(OH)H—NR⁴⁴R⁴⁵, C₁-C₆ alkylene-C(OH)H—C₁-C₆alkylene-NR⁴⁴R⁴⁵, —C(O)N(R^{N})—C₁-C₆alkylene-NR²⁴R²⁵, C₁-C₆alkylene-C(O)NR²⁴R²⁵ or C₁-C₆alkylene-CO₂R³⁷; each R³⁷ is independently H or C₁-C₆ alkyl; R⁴⁴ and R⁴⁵ are each independently selected from a H; polyhydroxyl group; substituted polyhydroxyl group; —C(O)-polyhydroxyl group; and substituted —C(O)-polyhydroxyl group, provided that both R⁴⁴ and R⁴⁵ are not H; n20 is 2 to 26; n21 is 1 to 26; and n25 is 1 or

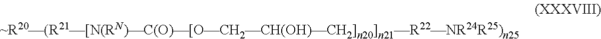

or a stereoisomer or salt thereof, wherein: R²⁰ is an attachment group to site R^{b}, or to the enzyme-cleavable group; R²¹ and R²² are each independently a bond or C₁-C₃ alkylene groups; R^{N} is H or C₁-C₄alkyl; R²⁴ and R²⁵ are each independently selected from a H; polyhydroxyl group; and substituted polyhydroxyl group, provided that R²⁴ and R²⁵ are not both H; n20 is 2 to 26; n21 is 1 to 4; and n25 is 1, 2 or 3;

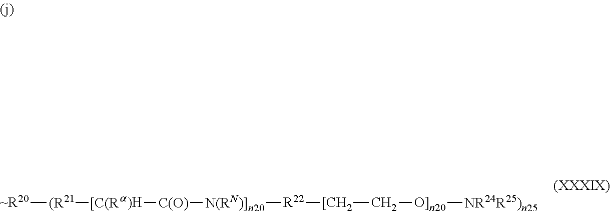

or a stereoisomer or salt thereof, wherein: R²⁰ is an attachment group to site R^{b}, or to the enzyme-cleavable group; R²¹ and R²² are each, independently, a bond, C₁-C₃ alkylene, —C₁-C₃alkylene-[O—CH₂—CH₂-]ₙ₂₀, —[CH₂—CH₂—O]ₙ₂₀—C₁-C₃alkylene- or —C₁-C₃alkylene-[O—CH₂—CH₂-]ₙ₂₀-C(O)—; each R^{α} is independently H or —R²²—NR²⁴R²⁵; each R^{N} is independently H, C₁-C₆ alkyl or —R²²—NR²⁴R²⁵; R²⁴ and R²⁵ are each independently selected from a H; polyhydroxyl group; substituted polyhydroxyl group; —C(O)-polyhydroxyl group; substituted —C(O)-polyhydroxyl group; substituted —C(O)—C₁-C₈ alkyl; a chelator; and —C(O)—R²⁸, where R²⁸ is a Sugar unit of formula (XII) or (XIII), provided that R²⁴ and R²⁵ are not both H; each n20 is independently 0 to 26, with the proviso that at least one n20 is 2 to 26; and n25 is 1 or 2; and

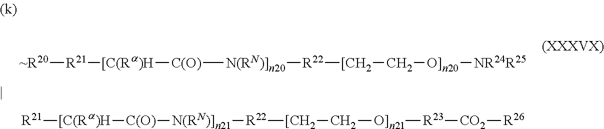

or a stereoisomer or salt thereof, wherein: R²⁰ is an attachment group to site R^{b}, or to the enzyme-cleavable group; R²¹, R²² and R²³ are each, independently, a bond, C₁-C₃ alkylene, —C₁-C₃alkylene-[O—CH₂—CH₂-]ₙ₂₀, —[CH₂—CH₂—O]ₙ₂₀—C₁-C₃alkylene- or —C₁-C₃alkylene-[O—CH₂—CH₂-]ₙ₂₀-C(O)—; each R^{α} is independently H or —R²²—NR²⁴R²⁵; each R^{N} is independently H, C₁-C₆ alkyl or —R²²—NR²⁴R²⁵; R²⁴ and R²⁵ are each independently selected from a H; polyhydroxyl group; substituted polyhydroxyl group; —C(O)-polyhydroxyl group; substituted —C(O)-polyhydroxyl group; substituted —C(O)—C₁-C₈ alkyl; a chelator; and —C(O)—R²⁸, where R²⁸ is a Sugar unit of formula (XII) or (XIII), provided that R²⁴ and R²⁵ are not both H; R²⁶ is H or C₁-C₆ alkyl; each n20 is independently 0 to 26, with the proviso that at least one n20 is 2 to 26; and each n21 is independently 0 to 26, with the proviso that at least one n21 is 2 to 26.

In some embodiments, provided are Linker compounds comprising a Polar group having a formula selected from the following, or a stereoisomer or salt thereof:

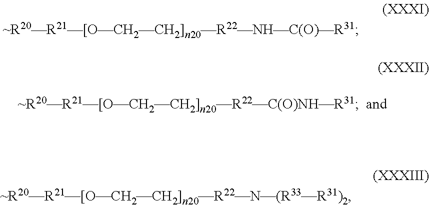

wherein: R²⁰ is an attachment group to site R^{b}, or to the enzyme-cleavable group; R²¹ and R²² are each, independently, a bond or C₁-C₃ alkylene groups; R³¹ is a branched polyethylene glycol chain, each branch having 1 to 26 ethylene glycol subunits and each branch having an R³⁵ at its terminus; R³³ is C₁-C₃ alkylene, —C₁-C₃ alkylene-C(O), —C(O)—C₁-C₃ alkylene or —C(O)—C₁-C₃ alkylene-C(O); R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; the wavy (˜) line indicates an attachment site to R²⁰; and n20 is 2 to 26.

In some embodiments, provided are Linker compounds comprising a Polar group formed from a precursor group selected from the following:

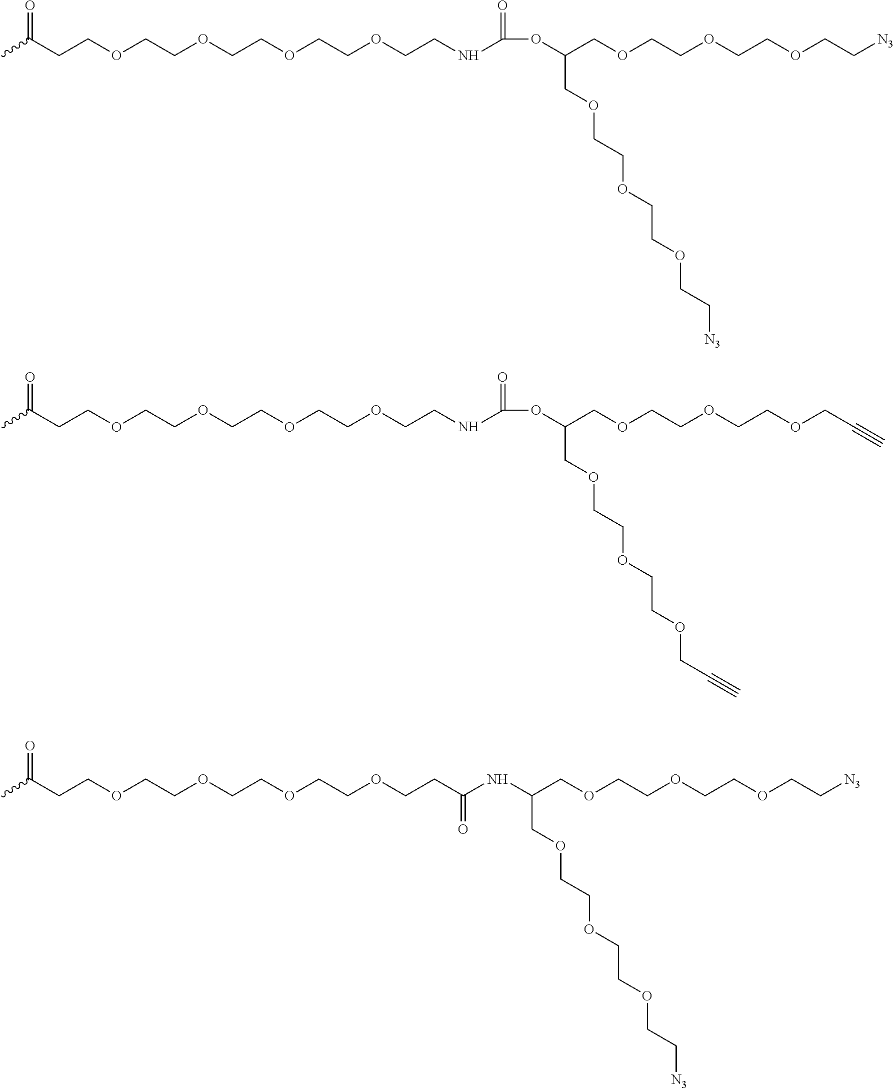

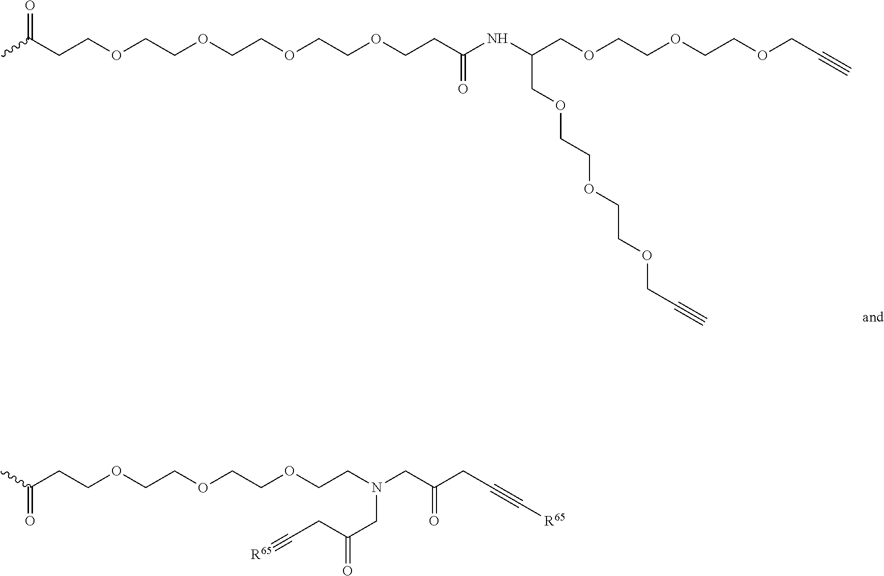

wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and the wavy line is an attachment site to R^{b}, or to the enzyme-cleavable group.

In some embodiments, the attachment site to R^{b}, or to the enzyme-cleavable group, as described above, is formed from a functional group of a precursor compound of the Polar group, said functional group selected from halo, aldehyde, carboxyl, amino, alkynyl, azido, hydroxyl, carbonyl, carbamate, thiol, urea, thiocarbamate, thiourea, sulfonamide, acyl sulfonamide, alkyl sulfonate, triazole, azadibenzocyclooctyne, hydrazine, carbonylalkylheteroaryl, and protected forms thereof.

In some embodiments, provided are Linker compounds comprising a Polar group having a formula:

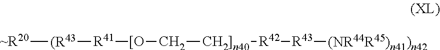

or a stereoisomer or salt thereof, wherein: R²⁰ is an attachment group to site R^{b}, or to the enzyme-cleavable group; R⁴¹ and R⁴² are each, independently, bond or C₁-C₆ alkylene; each R⁴³ is, independently, selected from a bond, C₁-C₁₂ alkylene, —OC₁-C₁₂ alkylene, —C(═O)—, —NR^{a}—C₁-C₁₂ alkylene, —C₁-C₁₂ alkylene-NR^{a}—, —C(O)—C₁-C₁₂ alkylene, —C₁-C₁₂ alkylene-C(O)—, —NR^{a}—C₁-C₁₂ alkylene-C(O)—, —C(O)—C₁-C₁₂ alkylene-NR^{a}—, —NR^{a}—C(O)—NR^{a}—, —NR^{a}—C(O)—, —NR^{a}—C(O)—C₁-C₁₂ alkylene, —C(O)—NR^{a}—C₁-C₁₂ alkylene, -heteroarylene, heteroaryl-C₁-C₁₂ alkylene, heteroaryl-C₁-C₁₂ alkylene-C(O)—, or —C(O)NR⁴⁶R⁴⁷, wherein each alkylene is optionally substituted with hydroxyl, SO₃H and/or oxo, R^{a} is H, C₁-C₆ alkyl, a polyhydroxyl group, or a substituted polyhydroxyl group, and one of R⁴⁶ and R⁴⁷ is H or C₁-C₁₂ alkylene and the other is C₁-C₁₂ alkylene, wherein one of the C₁-C₂ alkylenes is bound to NR⁴⁴R⁴⁵ at the nitrogen atom; R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, provided that R⁴⁴ and R⁴⁵ are not both H; n40 is 2 to 26; n41 is 1 to 6; and n42 is 1 to 6.

In some embodiments, provided are Linker compounds comprising a Polar group having a formula:

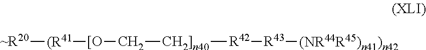

or a stereoisomer or salt thereof, wherein: R²⁰ is an attachment group to site R^{b}, or to the enzyme-cleavable group; R⁴¹ and R⁴² are each, independently, bond or C₁-C₆ alkylene; R⁴³ is selected from a bond, C₁-C₁₂ alkylene, —OC₁-C₁₂ alkylene, —C(═O)—, —NR^{a}—C₁-C₁₂ alkylene, —C₁-C₁₂ alkylene-NR^{a}—, —C(O)—C₁-C₁₂ alkylene, —C₁-C₁₂ alkylene-C(O)—, —NR^{a}—C₁-C₁₂ alkylene-C(O)—, —C(O)—C₁-C₁₂ alkylene-NR^{a}—, —NR^{a}—C(O)—NR^{a}—, —NR^{a}—C(O)—, —NR^{a}—C(O)—C₁-C₁₂ alkylene, C(O)—NR^{a}—C₁-C₁₂ alkylene, -heteroarylene, heteroaryl-C₁-C₁₂ alkylene, heteroaryl-C₁-C₁₂ alkylene-C(O)—, and —C(O)NR⁴⁶R⁴⁷, wherein each alkylene is optionally substituted with hydroxyl, SO₃H and/or oxo, R^{a} is H, C₁-C₆ alkyl, a polyhydroxyl group, or a substituted polyhydroxyl group and one of R⁴⁶ and R⁴⁷ is H or C₁-C₁₂ alkylene and the other is C₁-C₁₂ alkylene, wherein one of the C₁-C₂ alkylenes is bound to NR⁴⁴R⁴⁵ at the nitrogen atom; R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, provided that R⁴⁴ and R⁴⁵ are not both H; n40 is 1 to 26; n41 is 1 to 6; and n42 is 1 to 6.

In some embodiments, provided are Linker compounds comprising a Polar group having a formula:

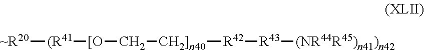

or a stereoisomer or salt thereof, wherein: R²⁰ is an attachment group to site R^{b}, or to the enzyme-cleavable group; R⁴¹ and R⁴² are each, independently, a bond or C₁-C₃ alkylene; R⁴³ is selected from a bond, C₁-C₆ alkylene, —OC₁-C₁₂ alkylene, —C(═O)—, —NR^{a}—C₁-C₁₂ alkylene, —C₁-C₆ alkylene-NR^{a}—, —C(O)—C₁-C₆ alkylene, —C₁-C₆ alkylene-C(O)—, —NR^{a}—C₁-C₆ alkylene-C(O)—, —C(O)—C₁-C₆ alkylene-NR^{a}—, —NR^{a}—C(O)—NR^{a}—, —NR^{a}—C(O)—, —NR^{a}—C(O)—C₁-C₆ alkylene, —C(O)—NR^{a}—C₁-C₁₂ alkylene, -heteroarylene, heteroaryl-C₁-C₆ alkylene, heteroaryl-C₁-C₆ alkylene-C(O)—, and —C(O)NR⁴⁶R⁴⁷, wherein each alkylene is optionally substituted with hydroxyl, SO₃H, and/or oxo, R^{a} is H, C₁-C₆ alkyl, a polyhydroxyl group, or a substituted polyhydroxyl group and one of R⁴⁶ and R⁴⁷ is H or C₁-C₆ alkylene and the other is C₁-C₁₂ alkylene, wherein one of the C₁-C₂ alkylenes is bound to NR⁴⁴R⁴⁵ at the nitrogen atom;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, provided that R⁴⁴ and R⁴⁵ are not both H; n40 is 1 to 16; n41 is 1 to 4; and n42 is 1 to 4.

In some embodiments, R²⁰ as described above is formed from a functional group of a precursor compound of the Polar group, said functional group selected from halo, aldehyde, carboxyl, amino, alkynyl, azido, hydroxyl, carbonyl, carbamate, thiol, urea, thiocarbamate, thiourea, sulfonamide, acyl sulfonamide, alkyl sulfonate, triazole, azadibenzocyclooctyne, hydrazine, carbonylalkylheteroaryl, or protected forms thereof.

In some embodiments, R²⁰ as described above comprises one of the following structures:

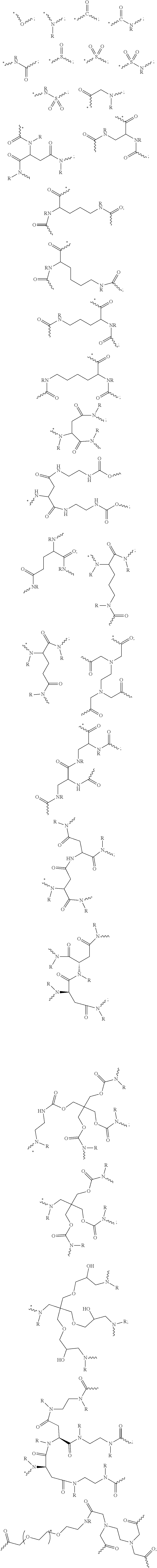

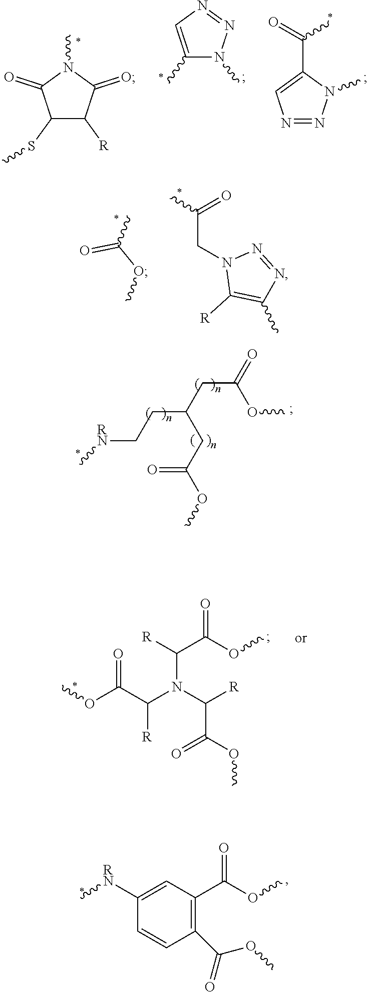

or a stereoisomer thereof, wherein R is H, C₁-C₆ alkyl or polyhydroxyl group, n is 0 to 12, the ( [symbol] *) indicates an attachment site to R^{b}, or to the enzyme-cleavable group, and the ( [symbol] ) indicated an attachment site to a remainder portion of the Polar group.

In some embodiments, R²⁰ as described above has one of the following structures:

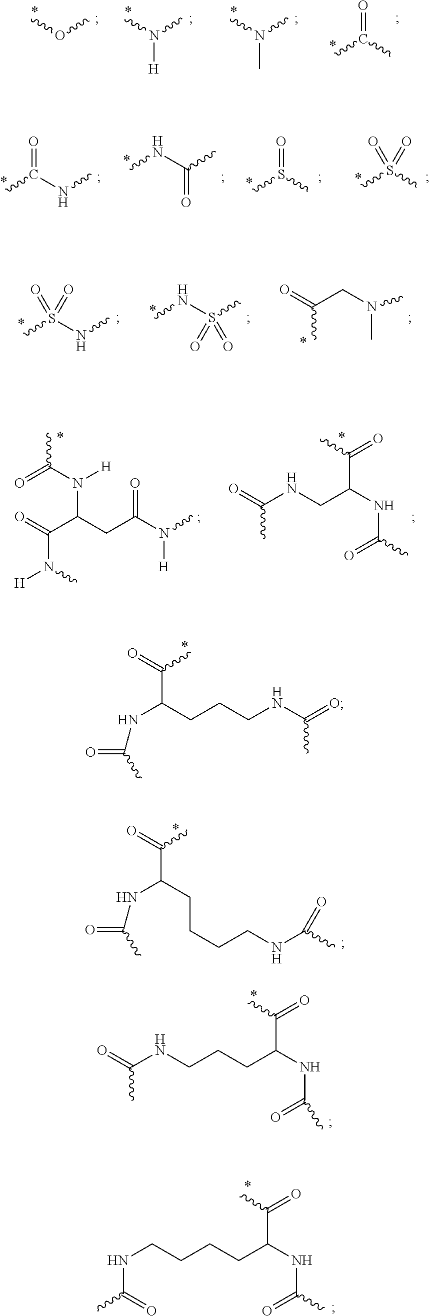

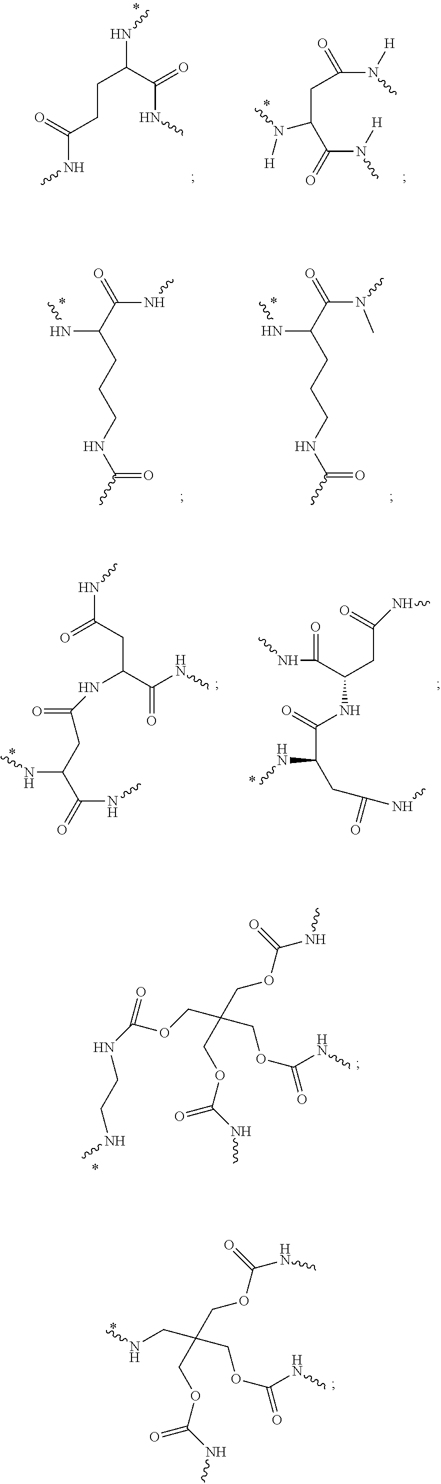

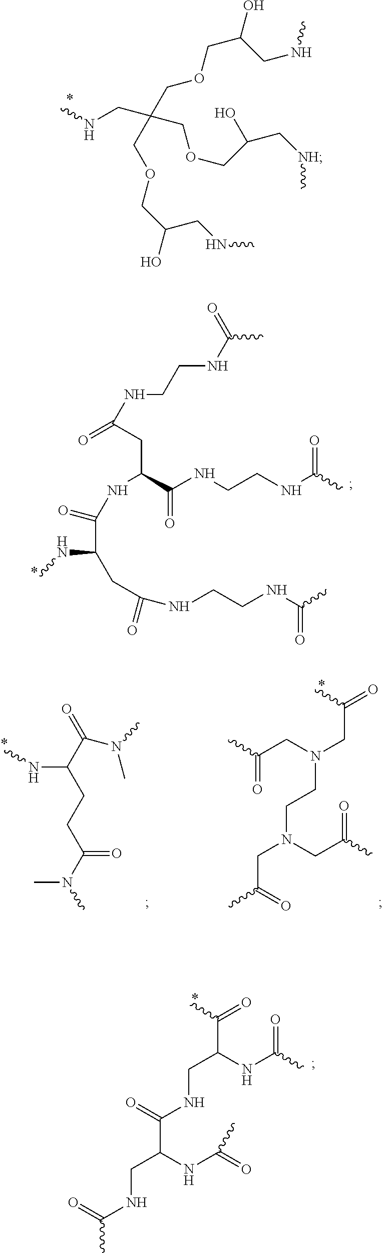

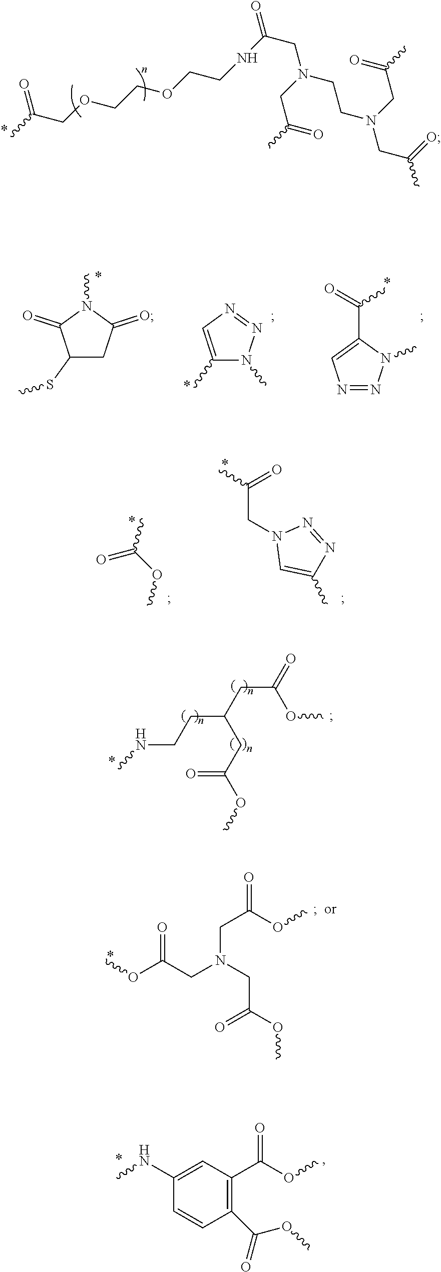

or a stereoisomer thereof, wherein n=0 to 12, the ( [symbol] *) indicates an attachment site to R^{b}, or to the enzyme-cleavable group, and the ( [symbol] ) indicates an attachment site to a remainder portion of the Polar group.

In some embodiments, R⁴³—(NR⁴⁴R⁴⁵)ₙ₄₁ as described above has one of the following structures:

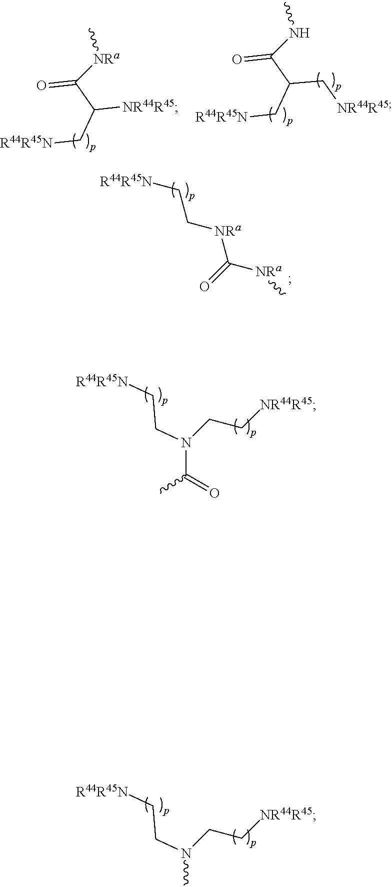

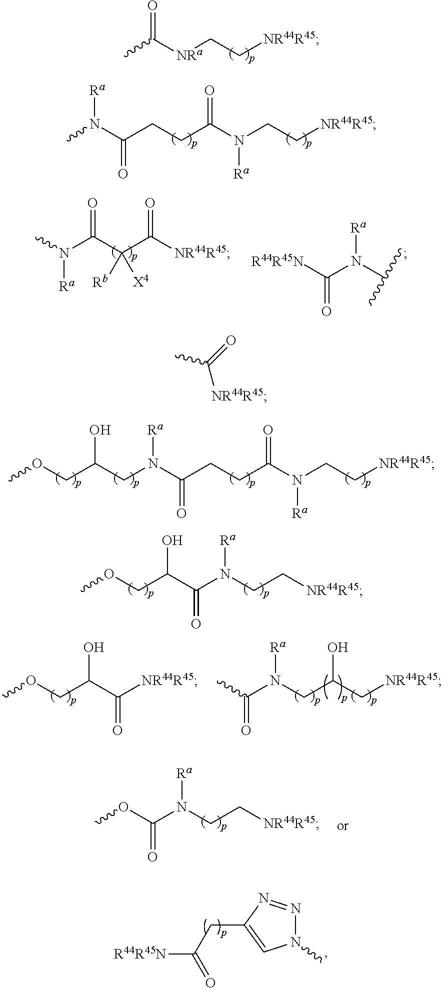

or a stereoisomer thereof, wherein R^{a} is H, C₁-C₆ alkyl, a polyhydroxyl group, or a substituted polyhydroxyl group; p is an integer from 1 to 6, and the ( [symbol] ) indicates the attachment site of R⁴³ to the remainder of the Polar group.

In some embodiments, R⁴³—(NR⁴⁴R⁴⁵)ₙ₄₁ as described above has one of the following structures:

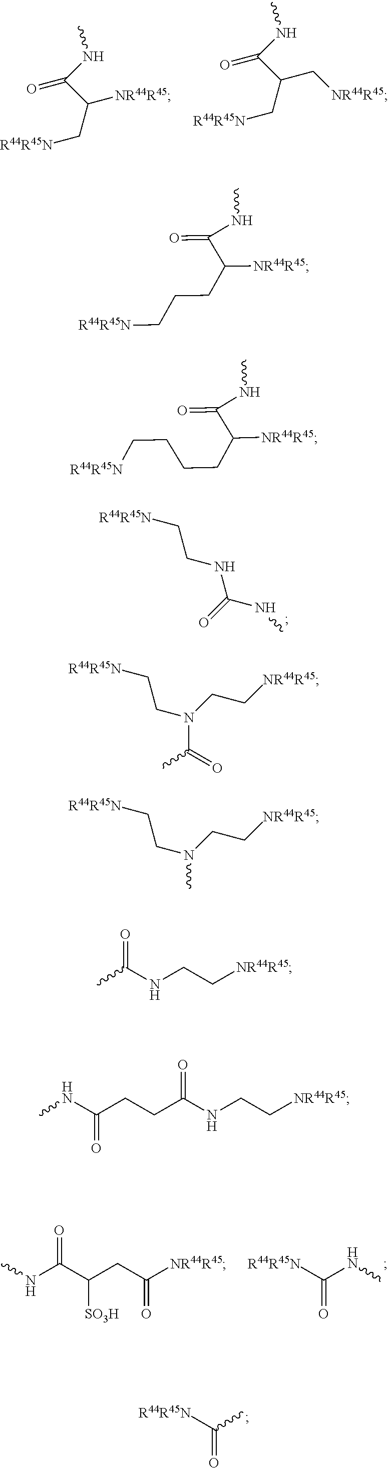

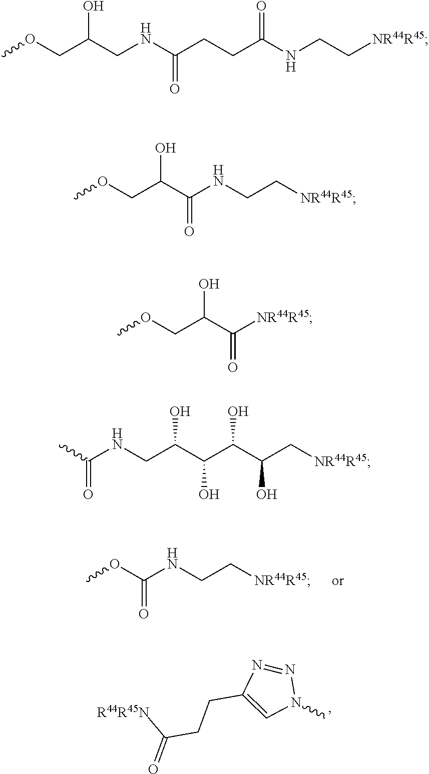

or a stereoisomer thereof, wherein the ( [symbol] ) indicates the attachment site of R⁴³ to the remainder of the Polar group.

In some embodiments, —NR⁴⁴R⁴⁵ as described above has one of the following structures:

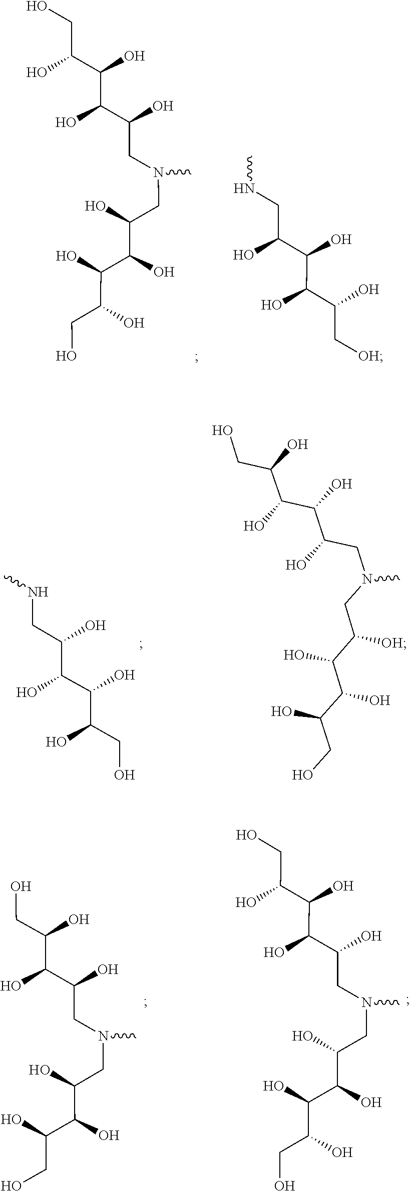

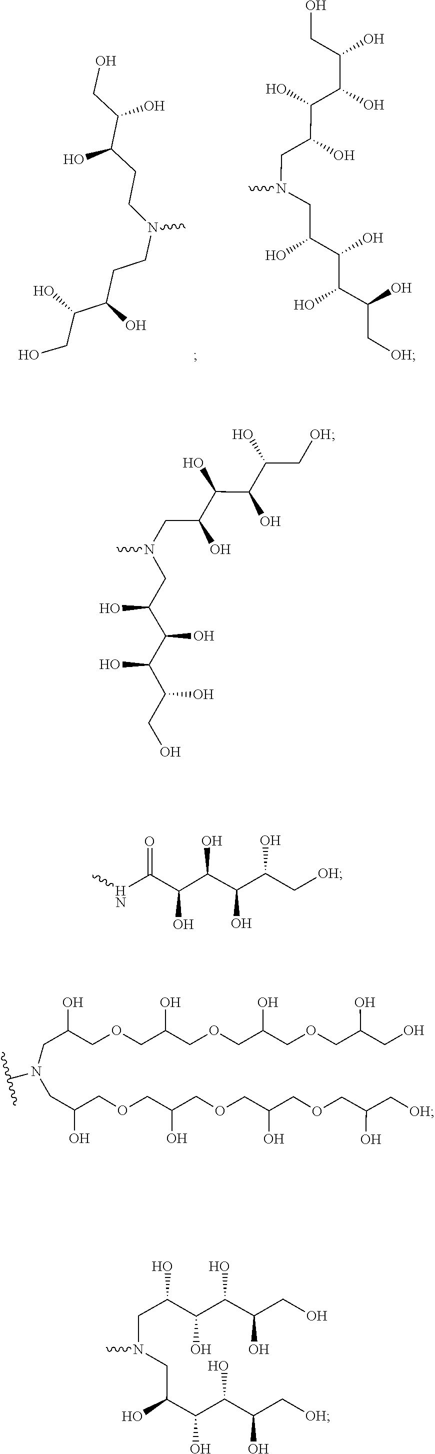

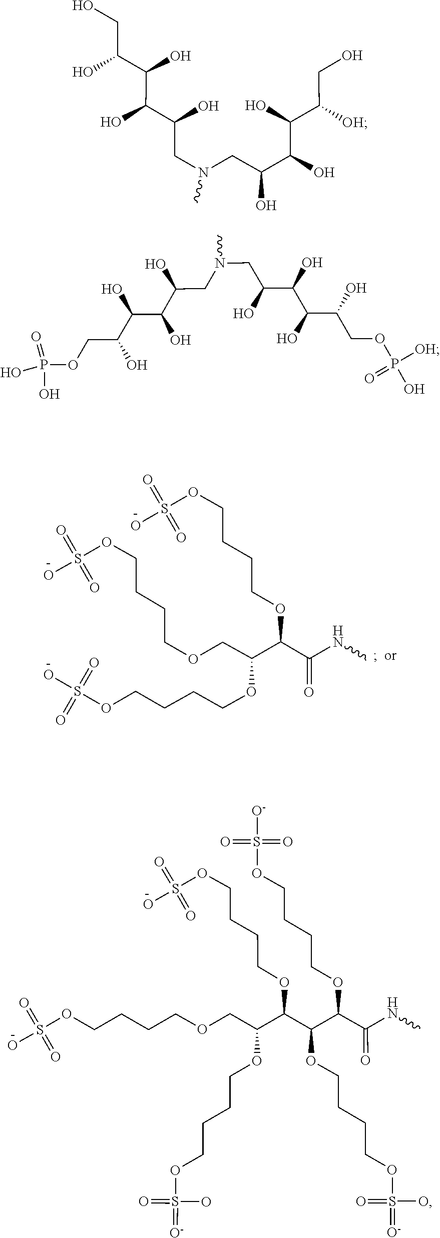

or a stereoisomer thereof, wherein the ( [symbol] ) indicates the attachment site of —NR⁴⁴R⁴⁵ to the remainder of the Polar group.

In some embodiments, provided are Linker compounds comprising a Polar group having one of the following structures prior to attachment to the Linker Unit:

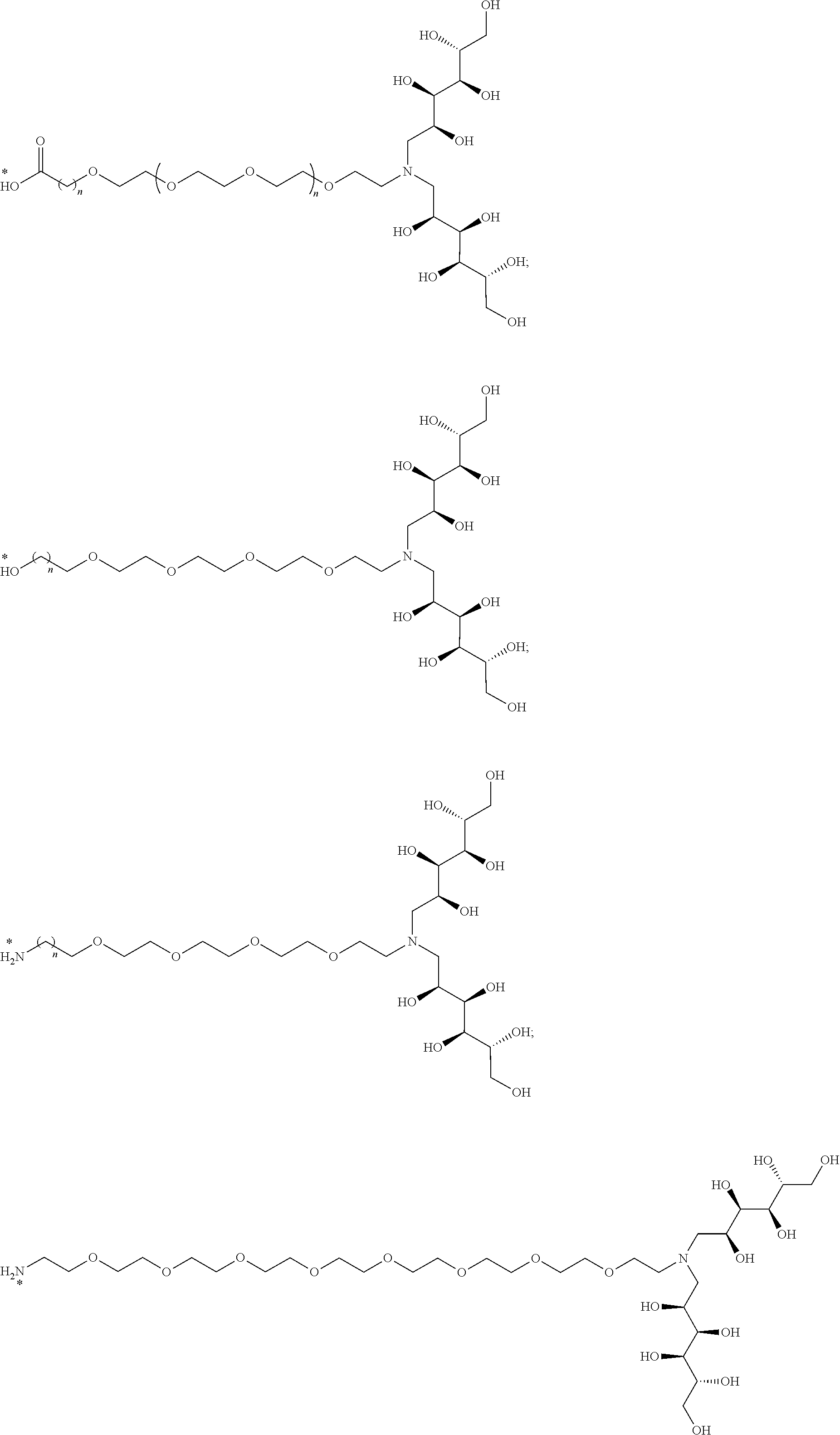

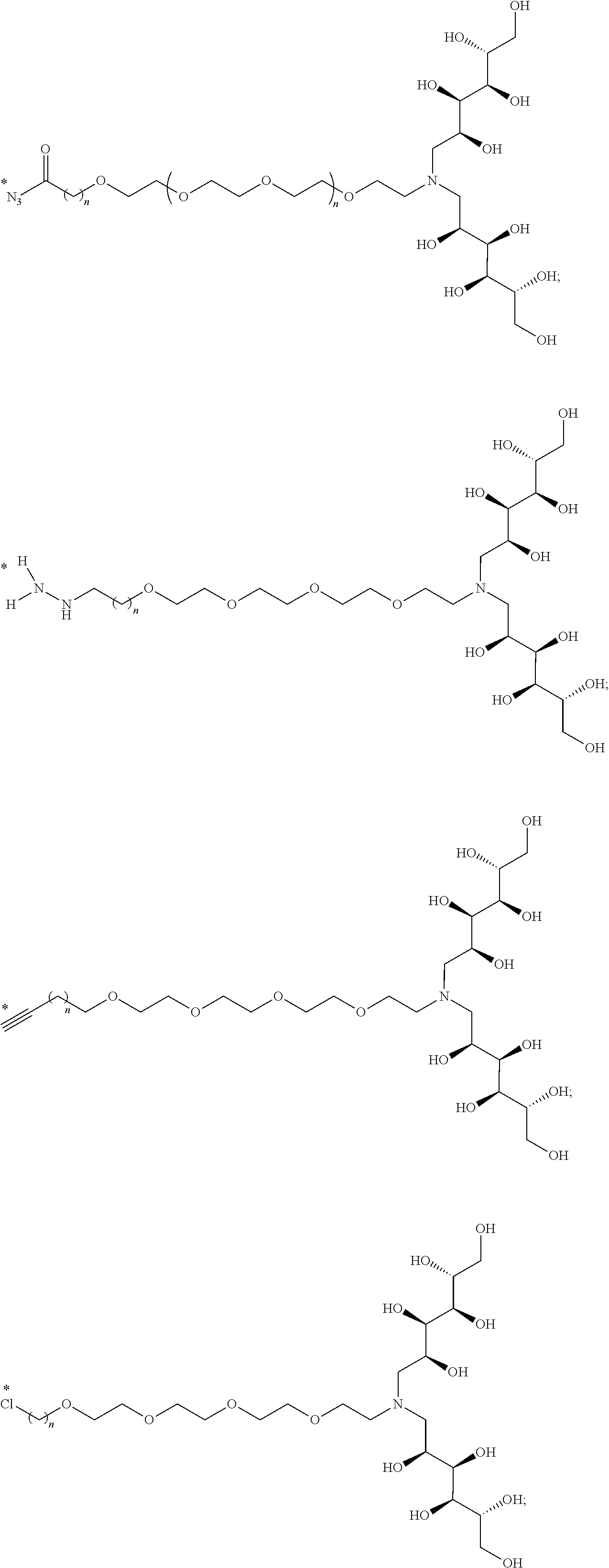

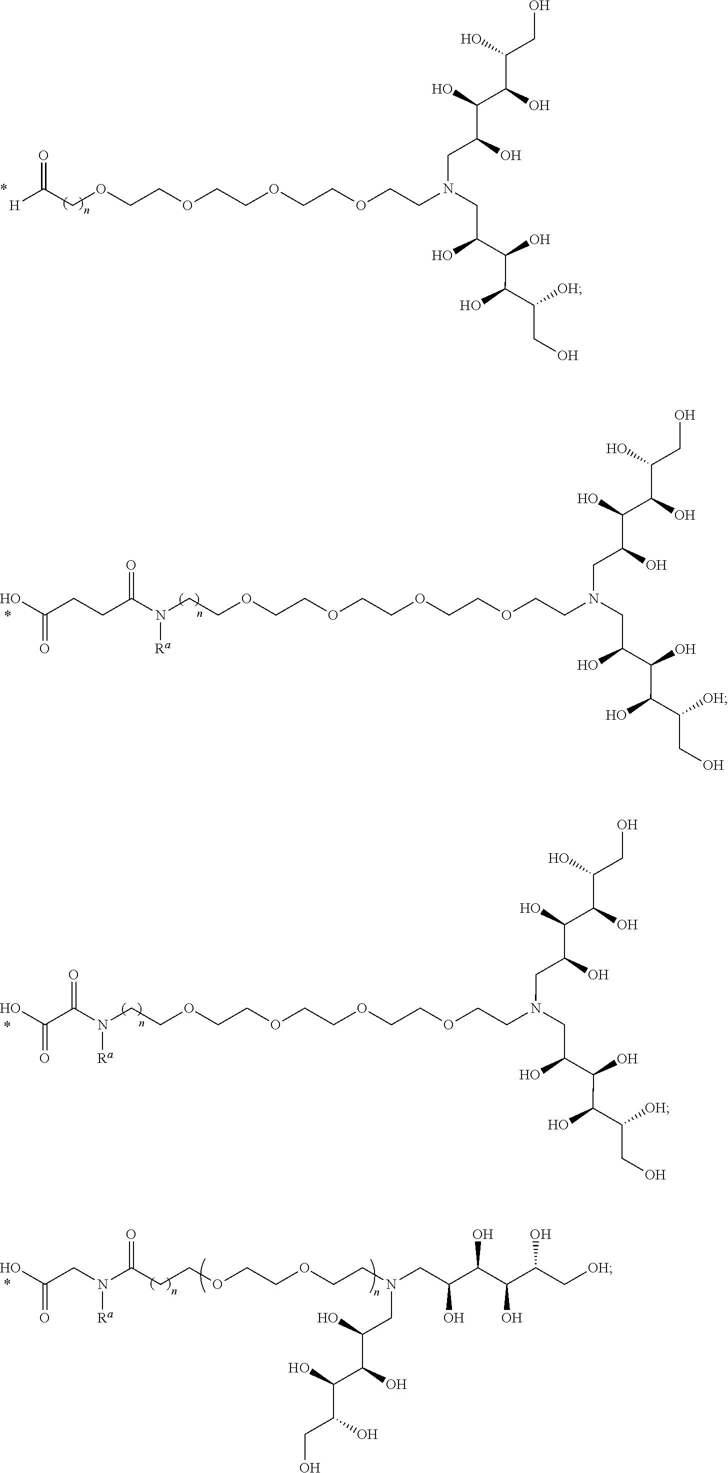

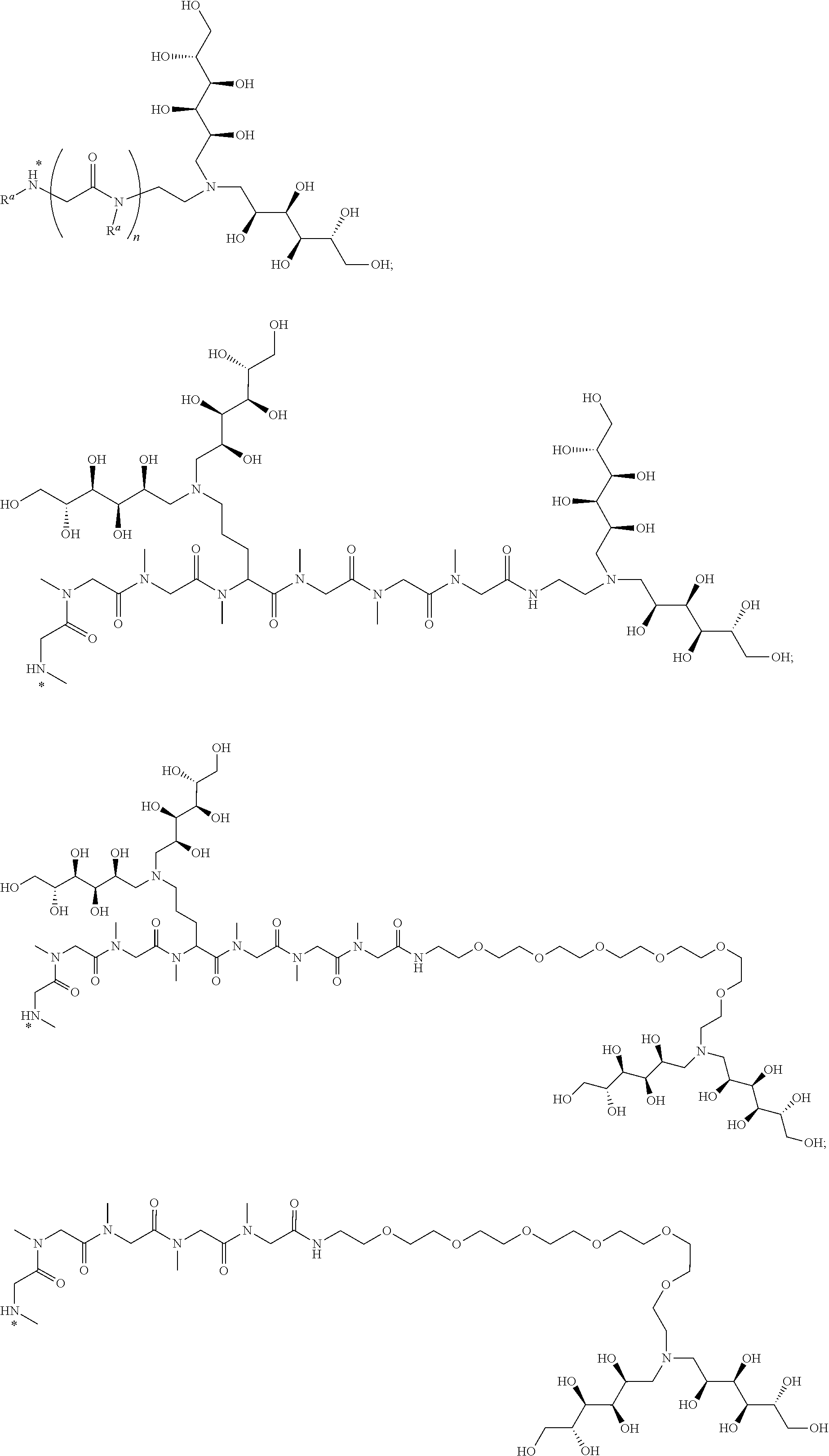

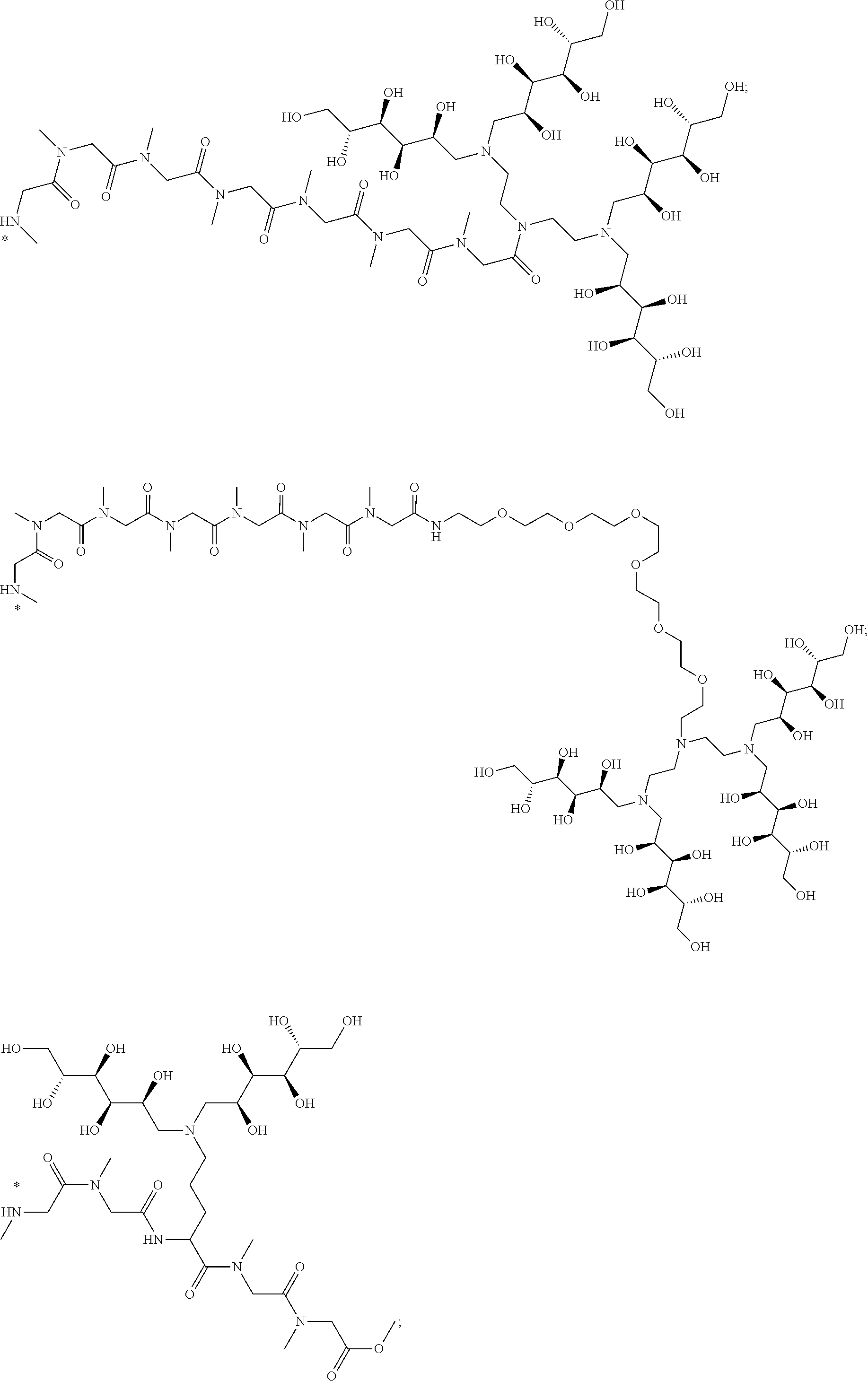

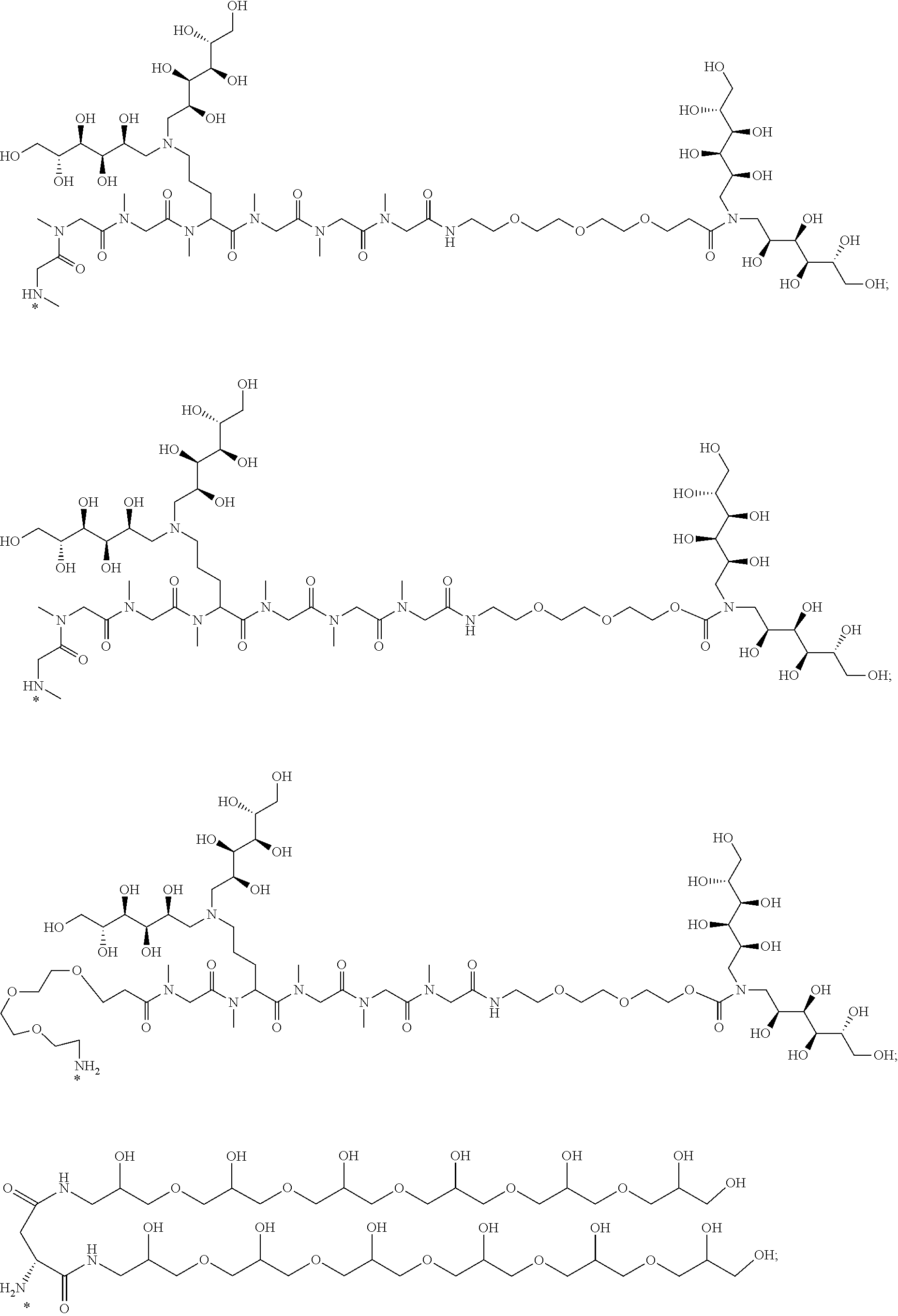

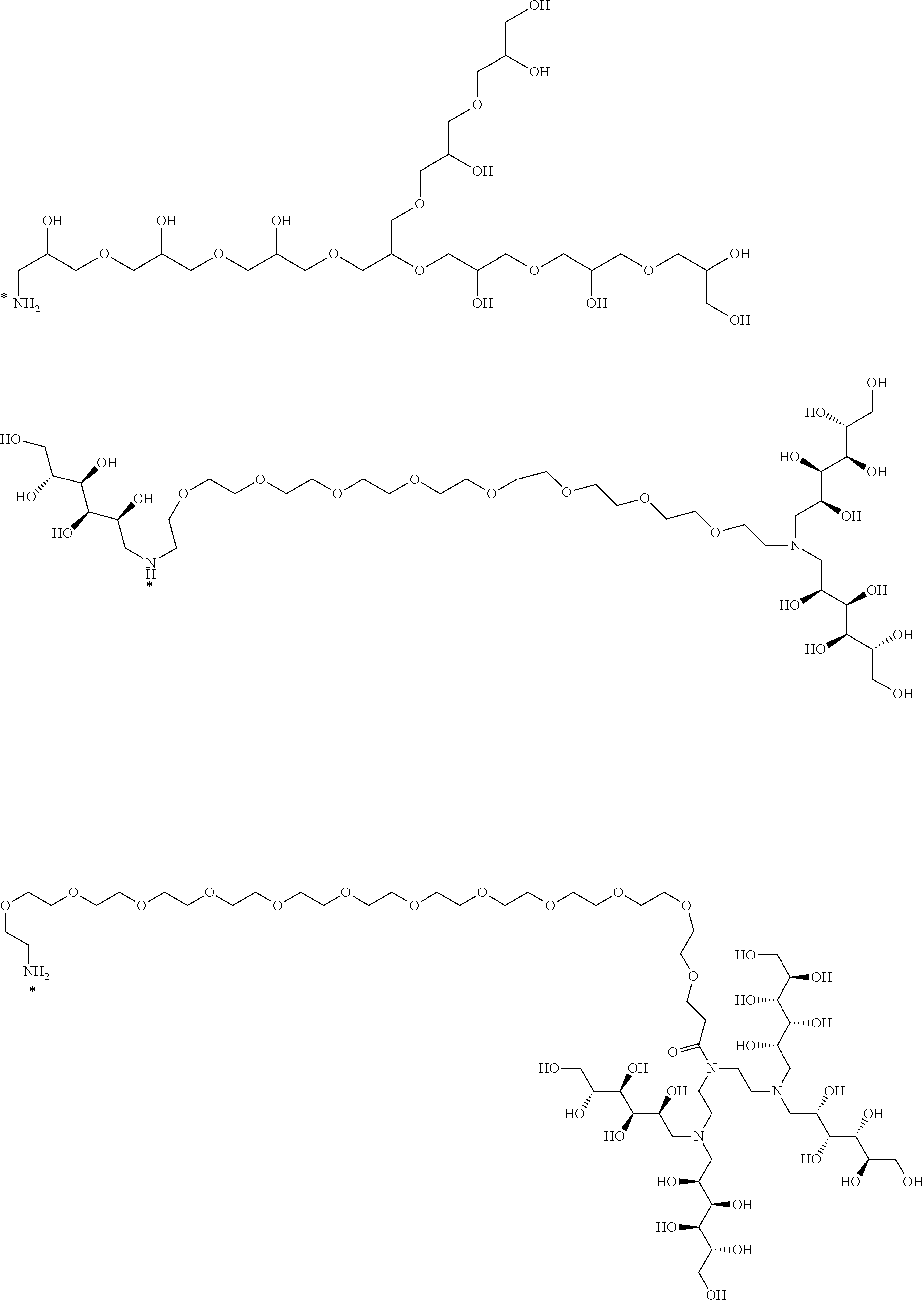

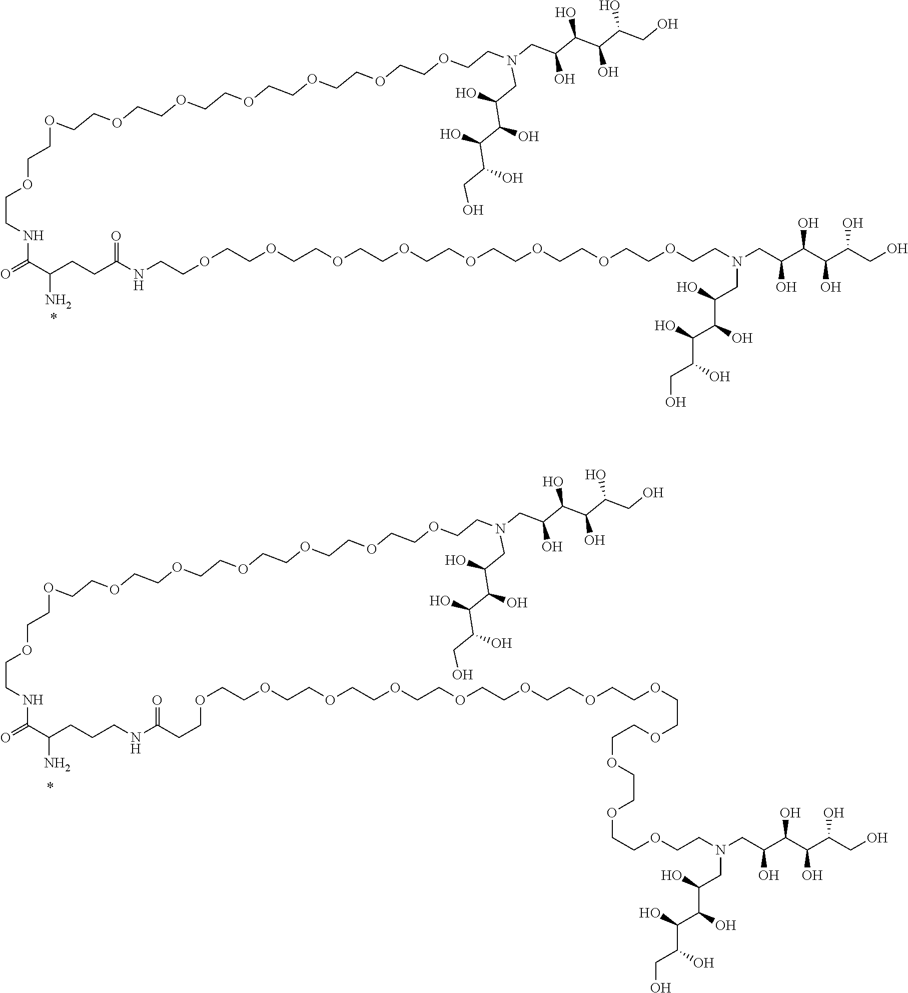

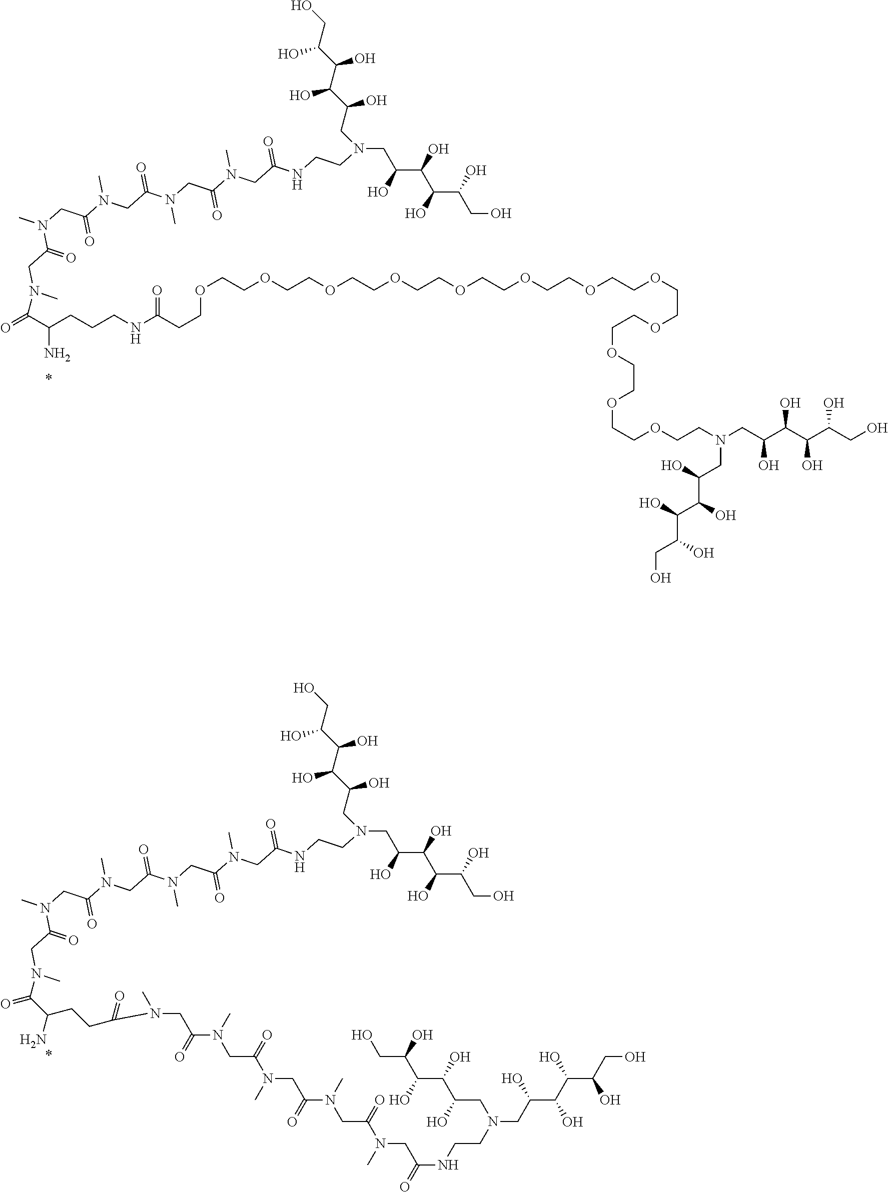

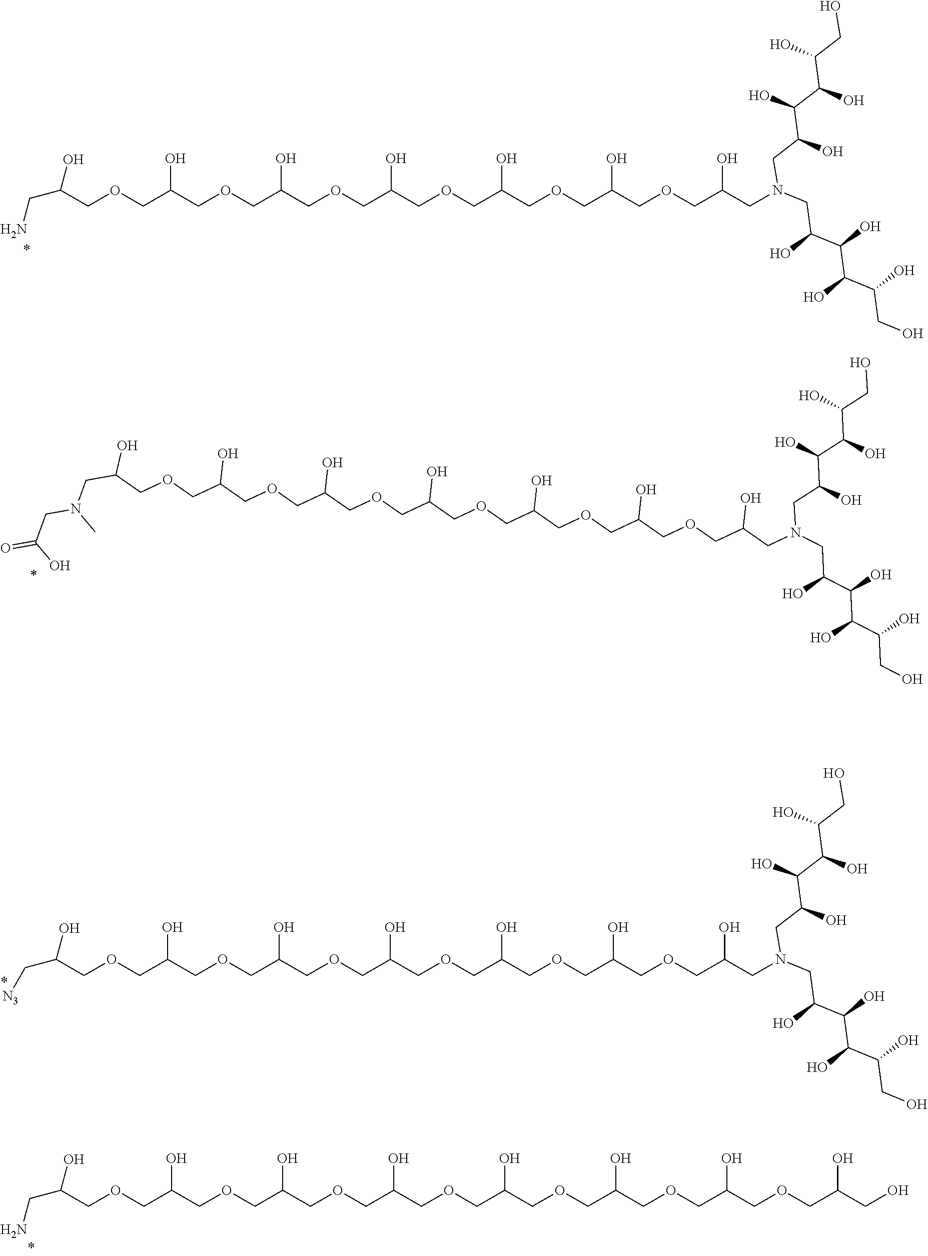

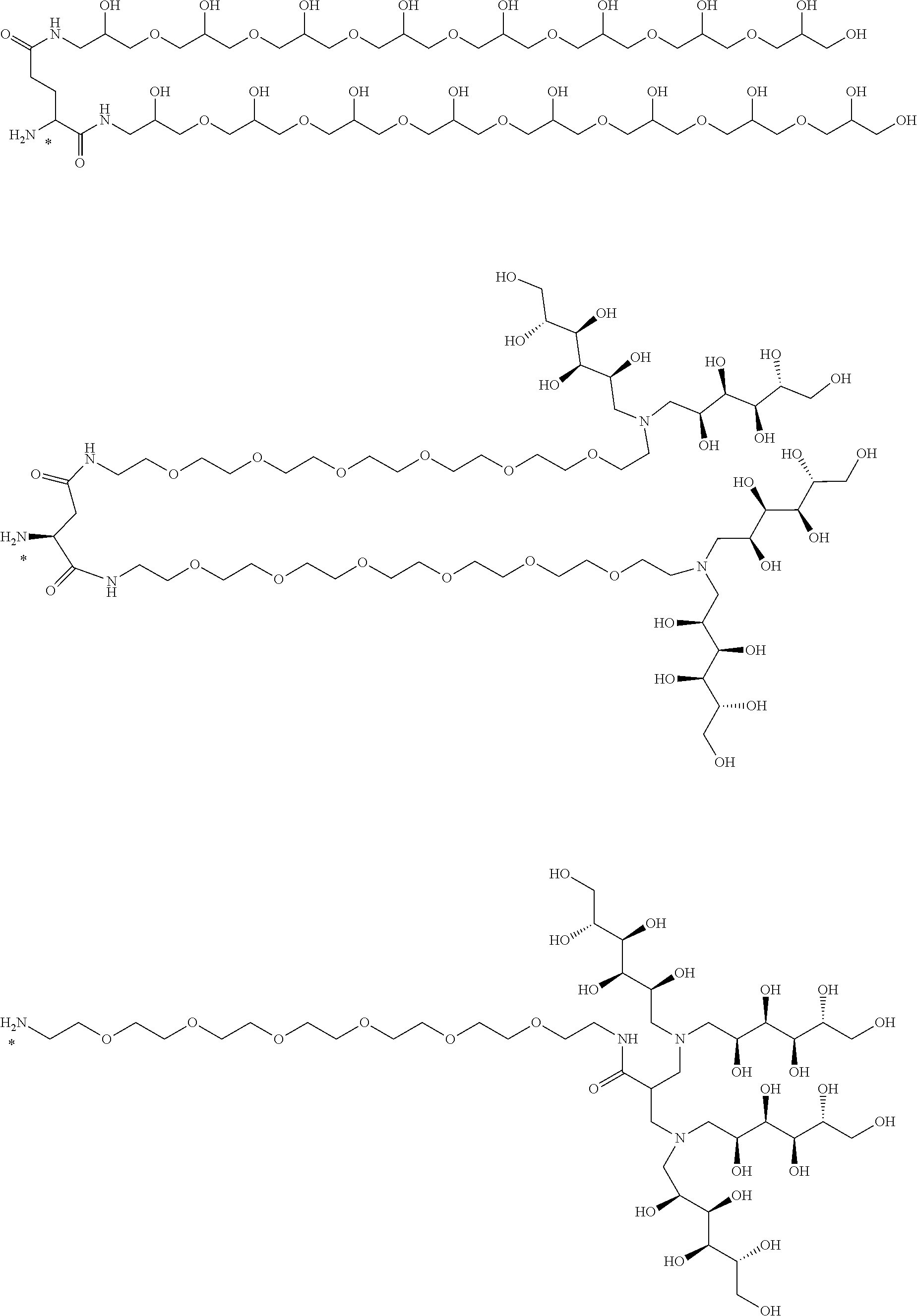

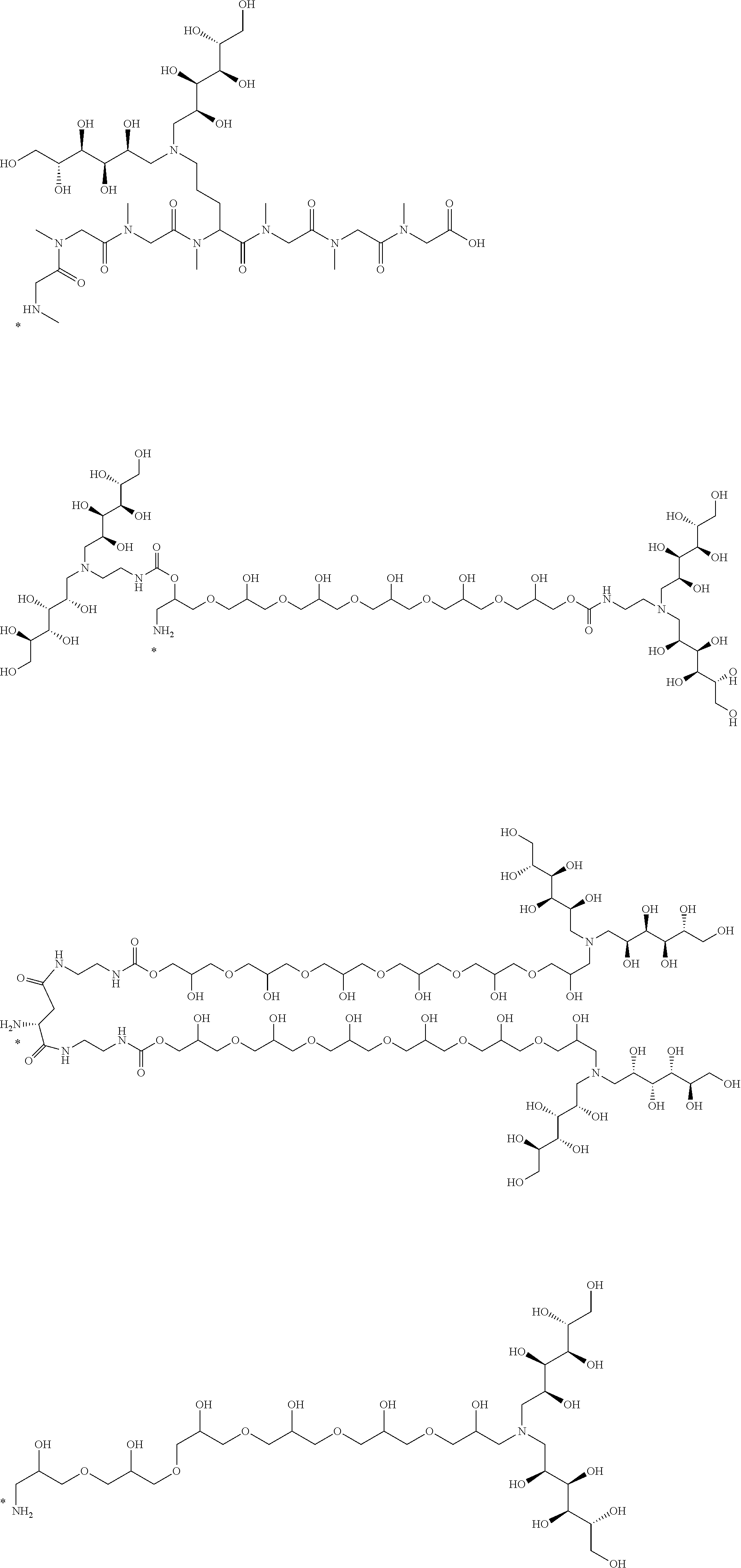

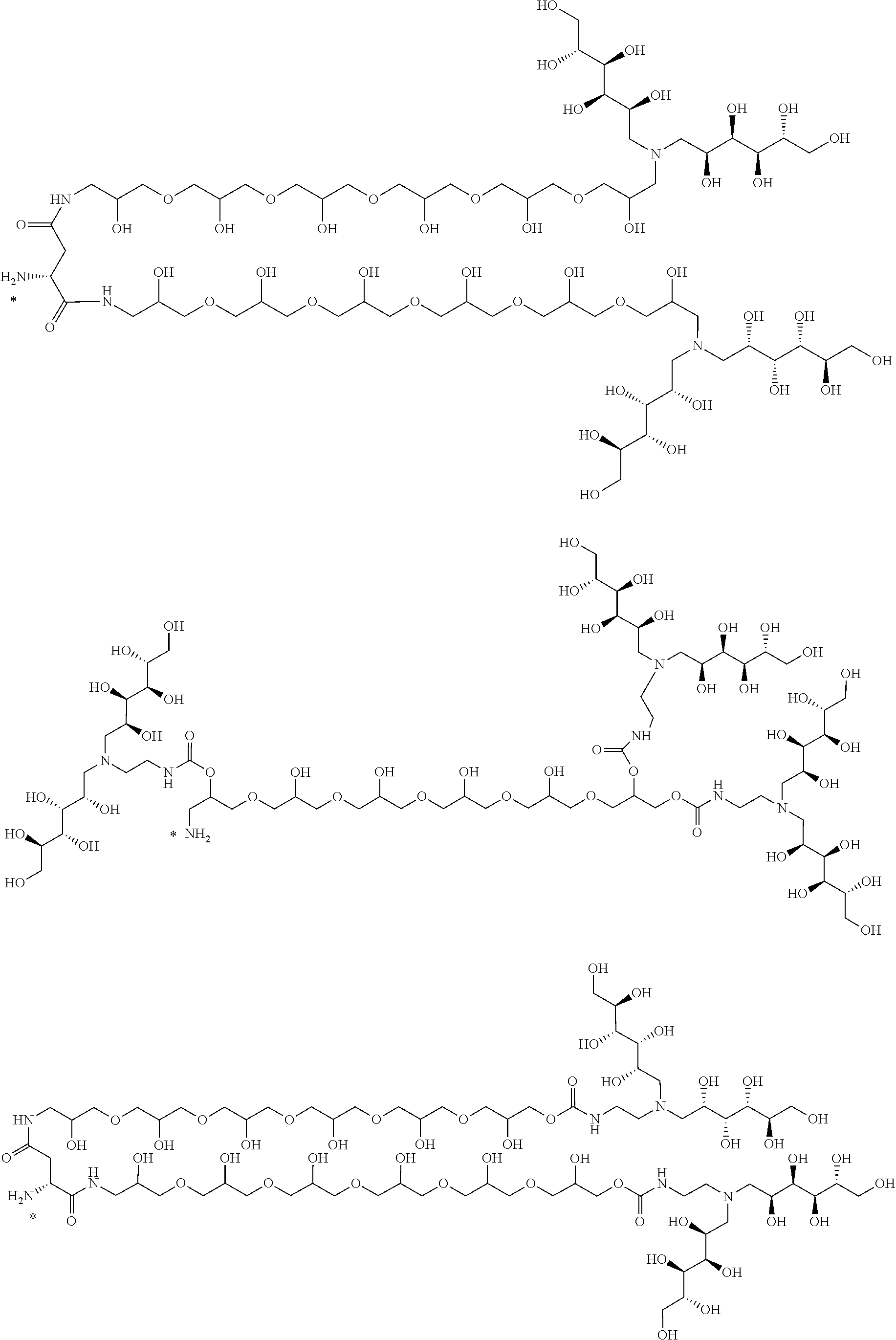

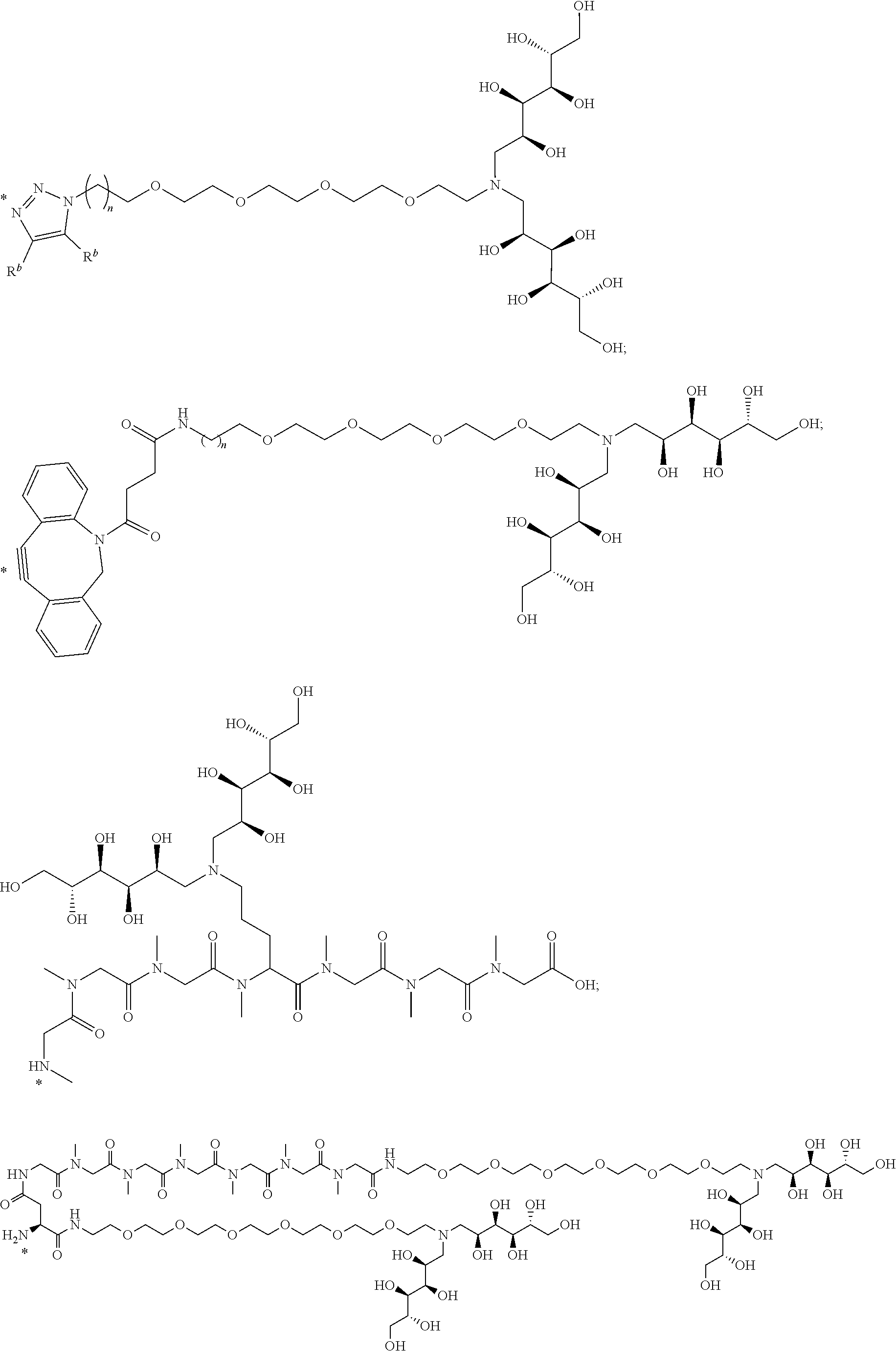

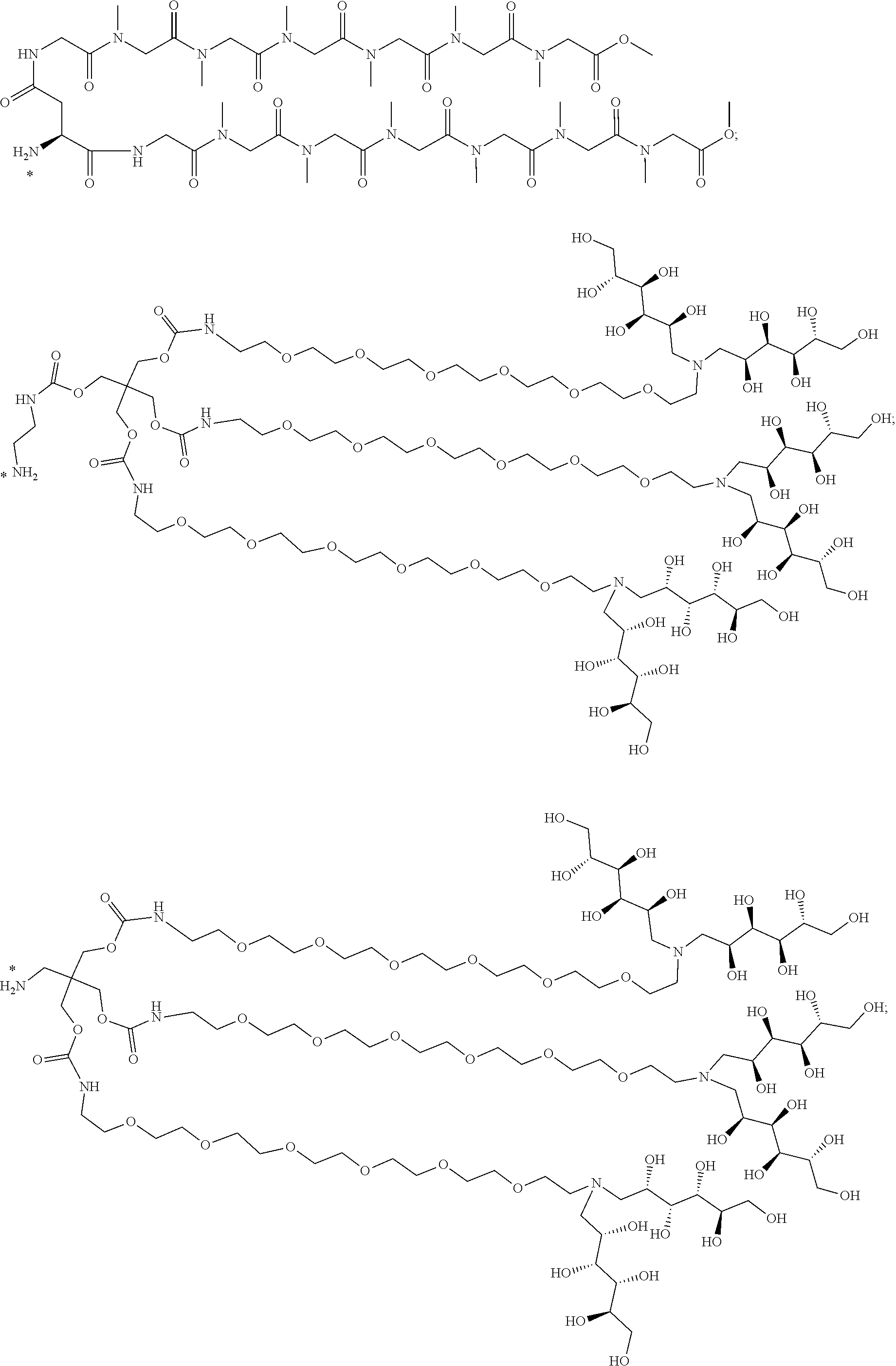

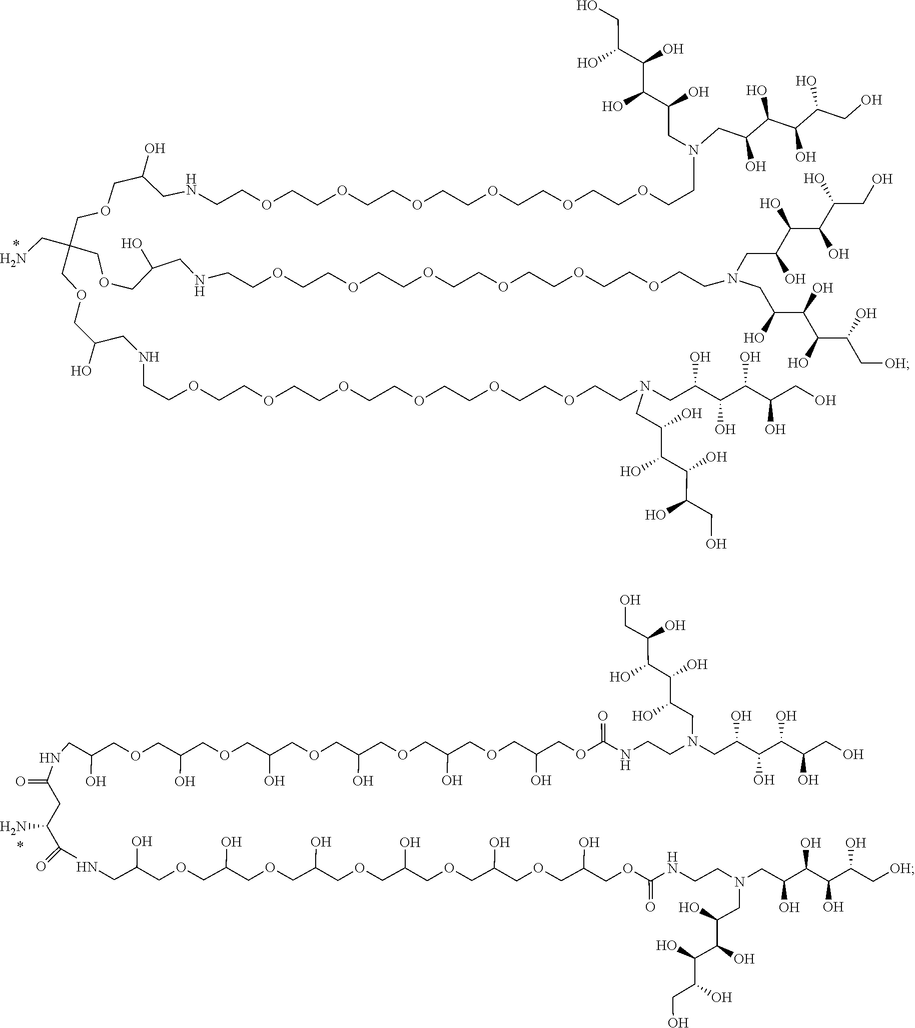

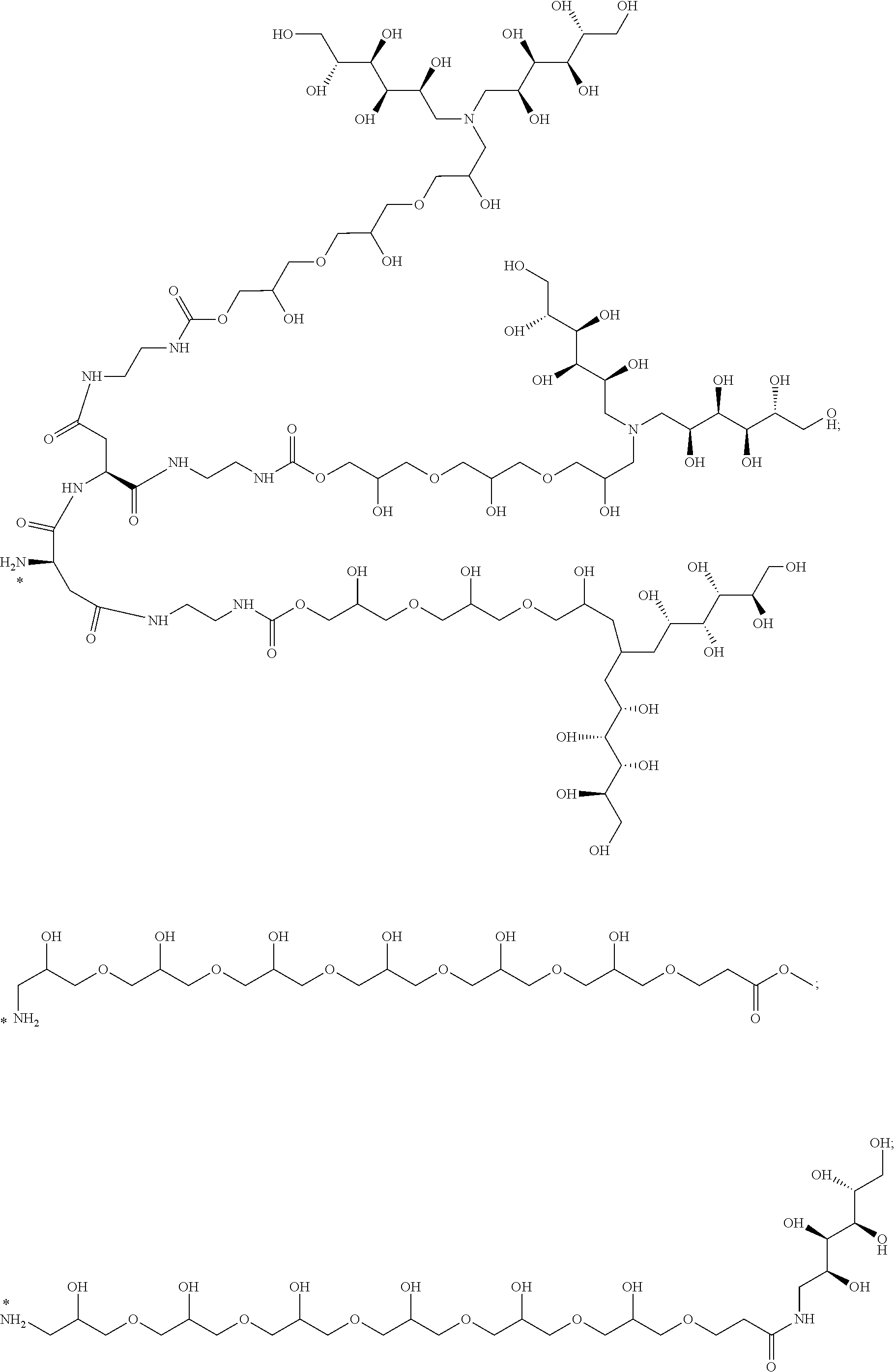

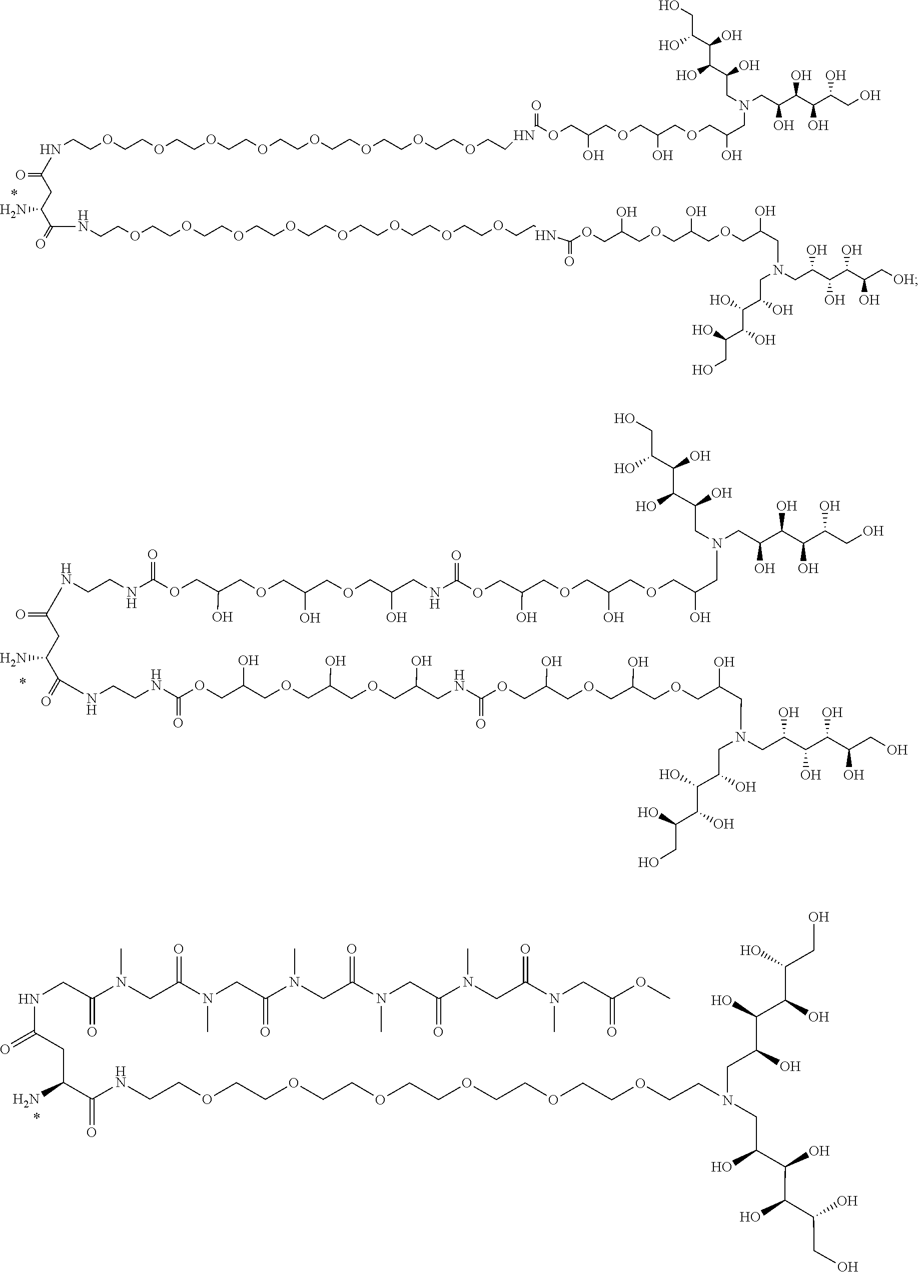

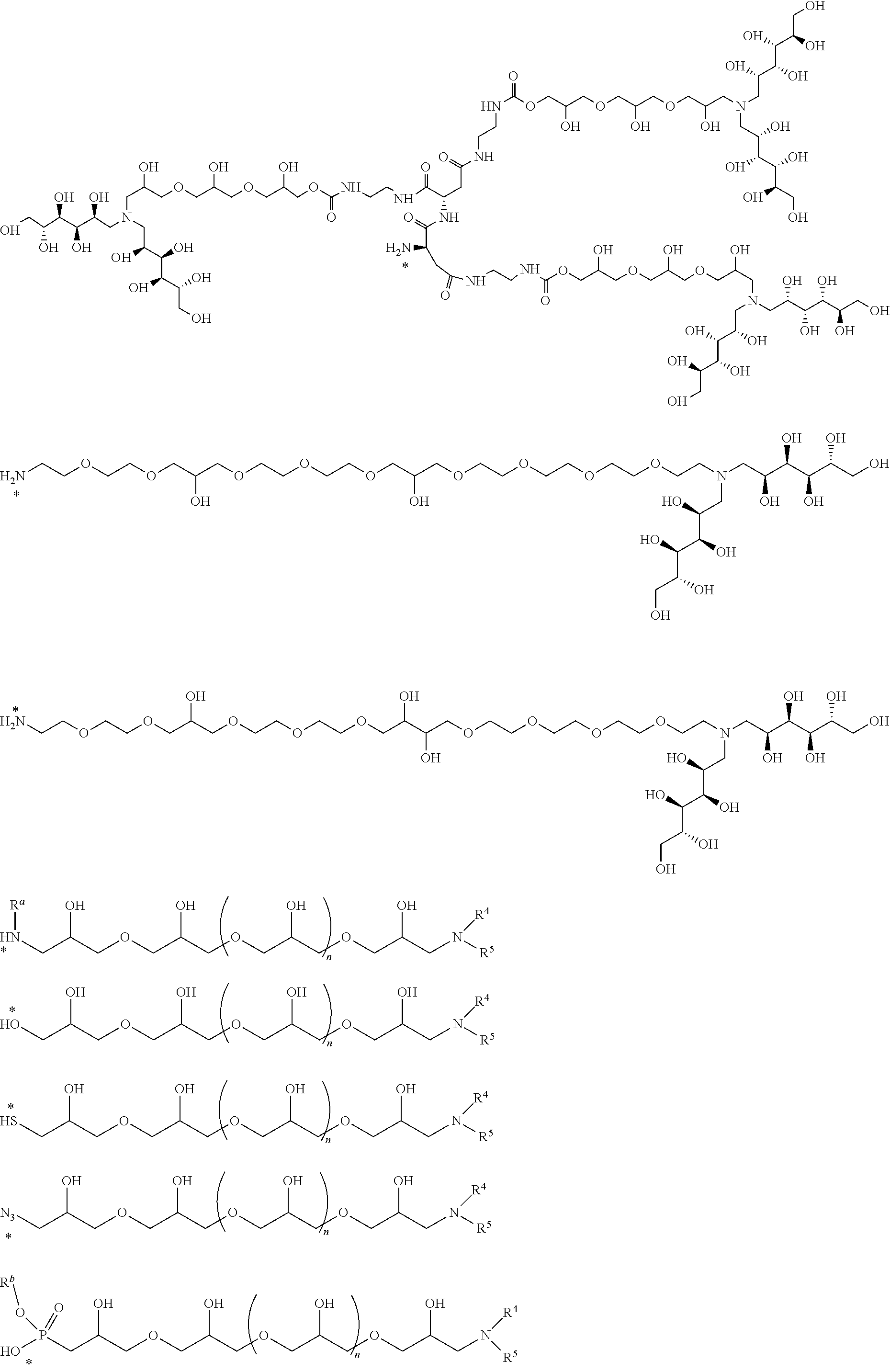

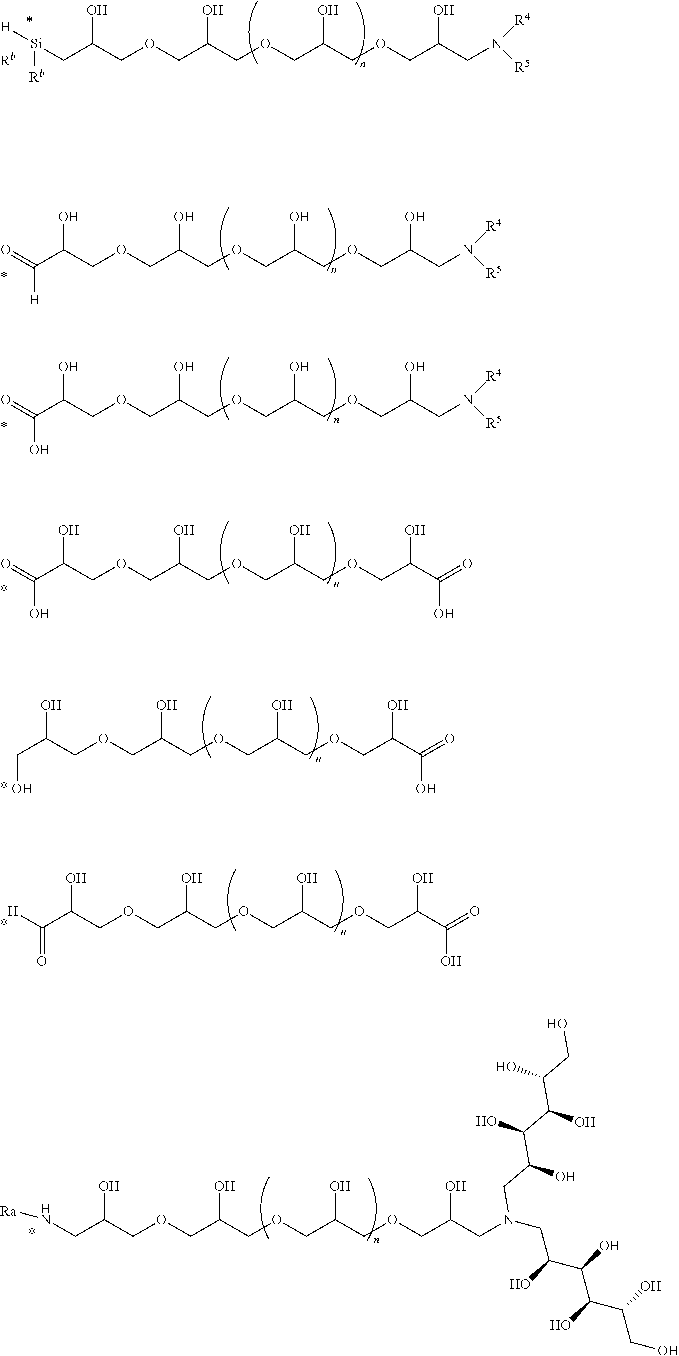

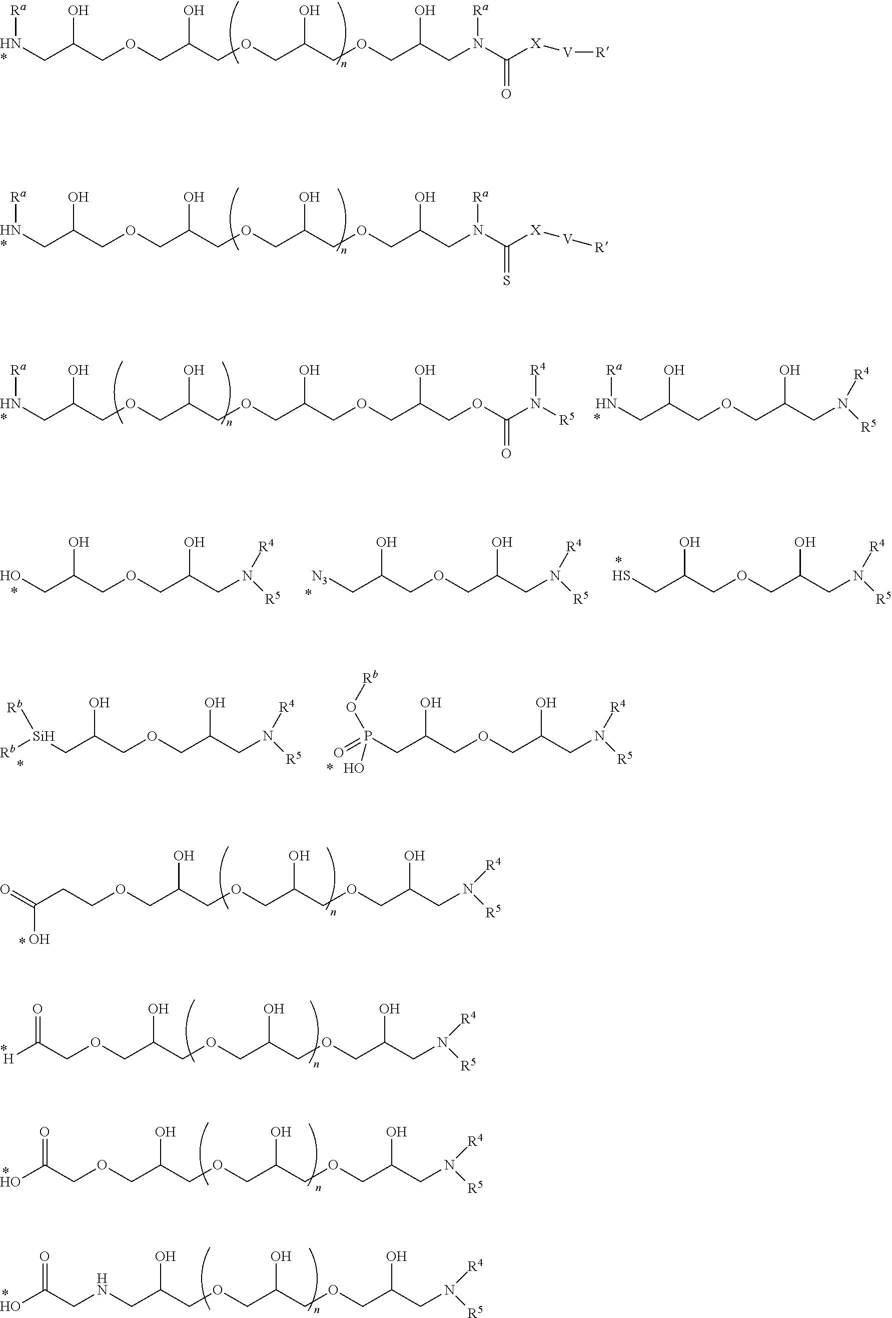

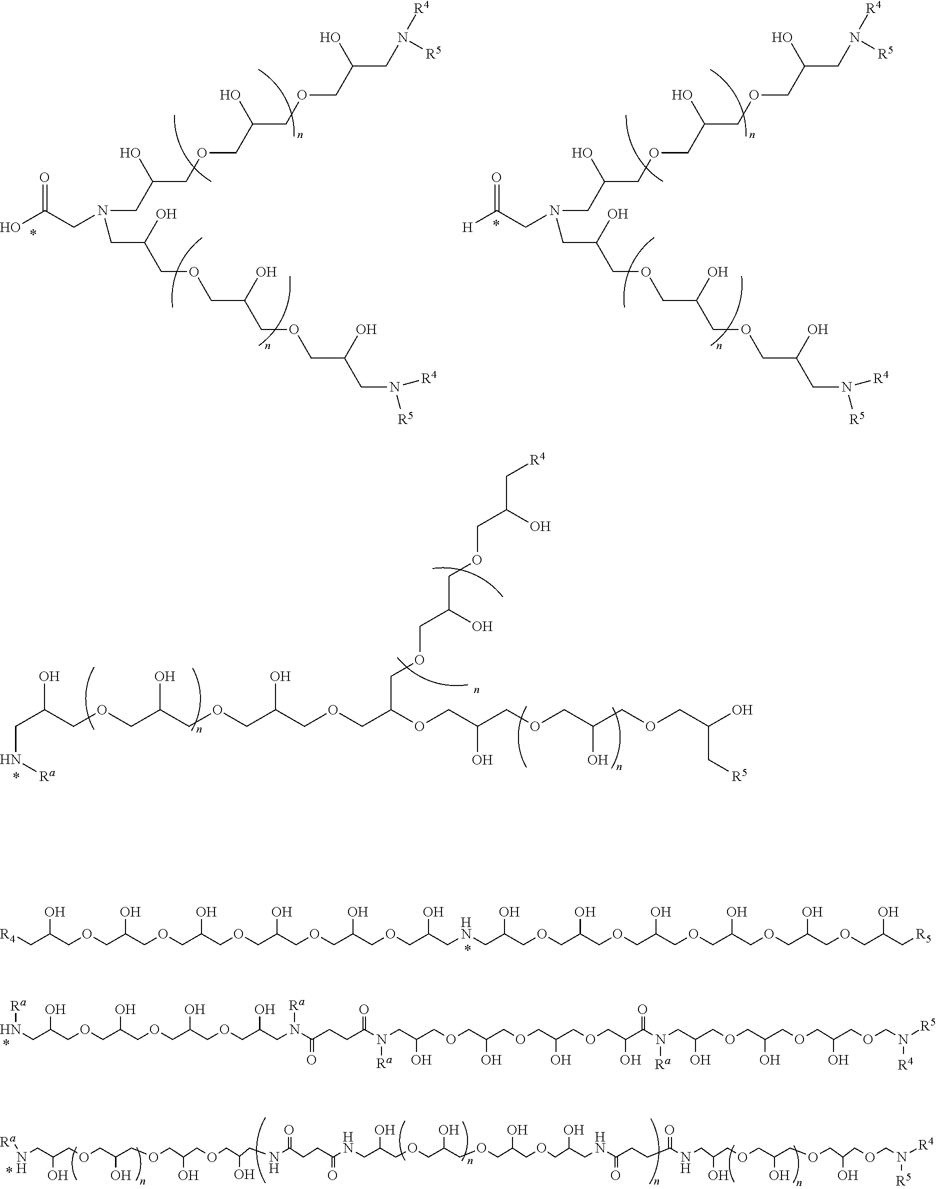

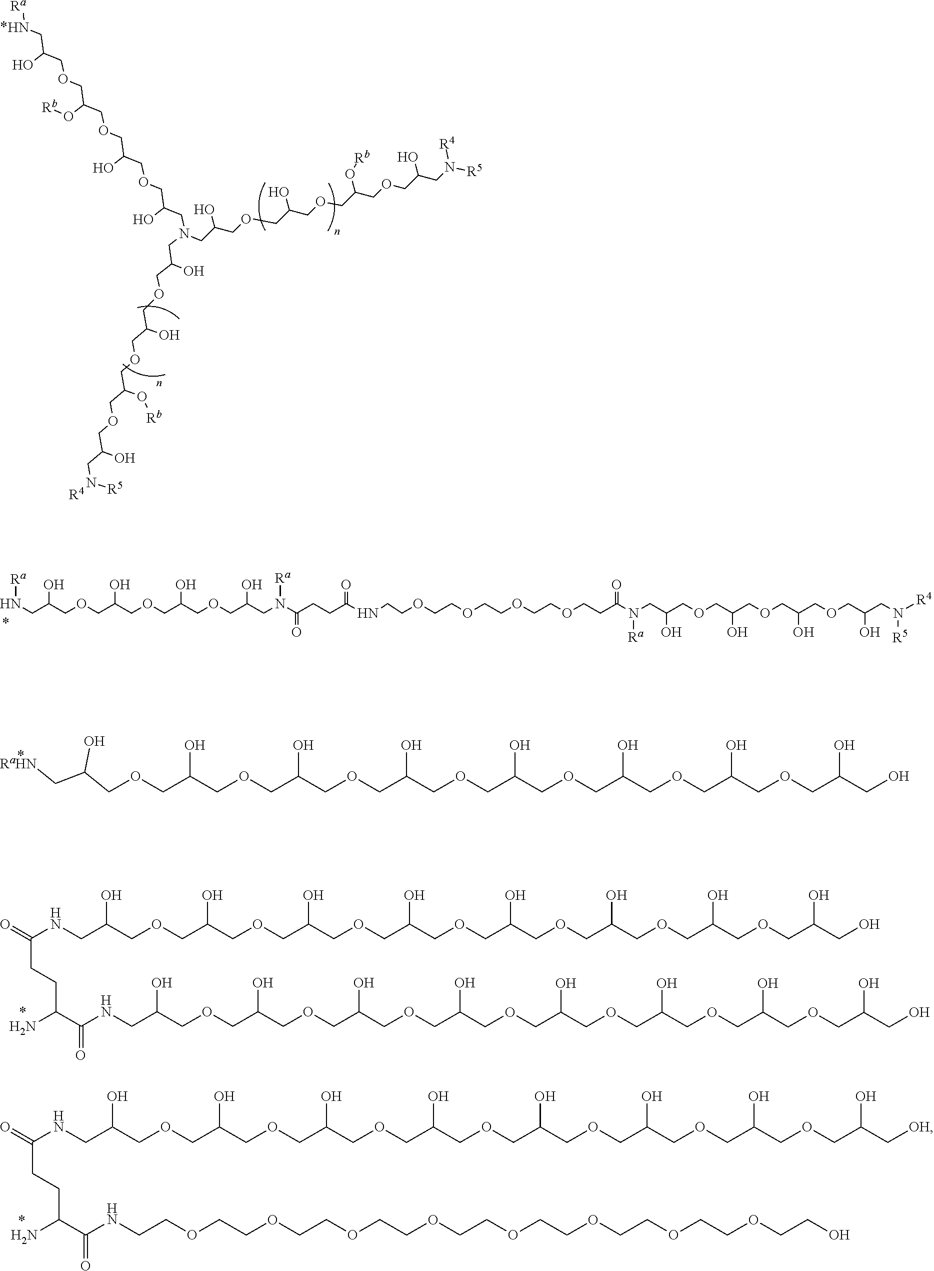

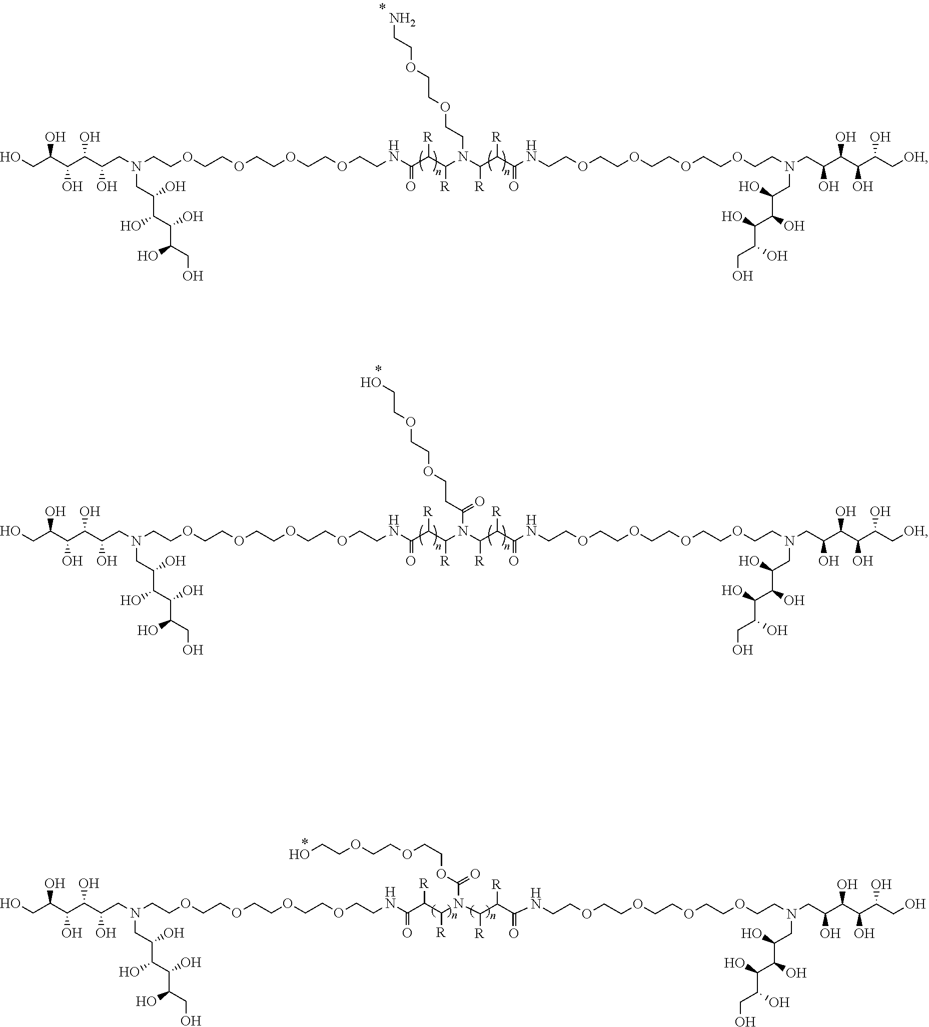

wherein: (*) indicates the attachment site to site R^{b}, or to the enzyme-cleavable group; each R is independently H or C₁-C₆ alkyl; R′ is H, C₁-C₆ alkyl, —N(R²⁴)(R²⁵) or —CO₂H; each n is independently 1 to 12; X is O, NR or —CH₂—; V is bond or C₁-C₆ alkyl; one of R²⁴ and R²⁵ is selected from a H; polyhydroxyl group; substituted polyhydroxyl group; —C(O)-polyhydroxyl group; substituted —C(O)-polyhydroxyl group; substituted —C(O)—C₁-C₈ alkyl; a chelator; and —C(O)—R²⁸, where R²¹ is a Sugar unit of formula (XII) or (XIII); and the other of R²⁴ and R²⁵ is selected from H; polyhydroxyl group; substituted polyhydroxyl group; —C(O)-polyhydroxyl group; substituted —C(O)-polyhydroxyl group; substituted —C(O)—C₁-C₈ alkyl; a chelator; and —C(O)—R²⁸, where R²⁸ is a Sugar unit of formula (XII) or (XIII); and polyethylene glycol, optionally having 1 to 24 ethylene glycol subunits, provided that R²⁴ and R²⁵ are not both H. In some embodiments of the previous embodiment, wherein: each R is independently H or C₁-C₆ alkyl; R′ is H, C₁-C₆ alkyl, —N(R²⁴)(R²⁵) or —CO₂H; each n is independently 1 to 12; X is O, NR or —CH₂—; V is bond or C₁-C₆ alkyl; one of R²⁴ and R²⁵ is selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; —C(O)—R²⁸, where R²⁸ is a Sugar unit of formula (XII) or (XIII); and the other of R²⁴ and R²⁵ is selected from H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; —C(O)—R²⁸, where R²⁸ is a Sugar unit of formula (XII) or (XIII); and polyethylene glycol, optionally having 1 to 24 ethylene glycol subunits; or —NR²⁴R²⁵ together from a C₃-C₈ heterocycle, provided that R²⁴ and R²⁵ are not both H.

In some embodiments, provided are Linker compounds comprising a Polar group having a formula selected

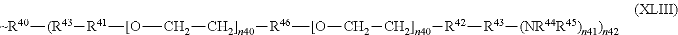

or a stereoisomer or salt thereof, wherein: R⁴⁰ is an attachment group to site R^{b}, or to the enzyme-cleavable group; R⁴¹ and R⁴² are each, independently, a bond or C₁-C₆ alkylene; each R⁴³ is, independently, selected from a bond, C₁-C₁₂ alkylene, —OC₁-C₁₂ alkylene, —C(═O)—, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)—, —C(O)—C₁-C₁₂ alkylene-NH—, —NH—C(O)—NH—, —NH—C(O)—, —NH—C(O)—C₁-C₁₂ alkylene, —C(O)—NH—C₁-C₁₂ alkylene, C₁-C₁₂alkylene-NH—C(O)—, -heteroarylene, heteroaryl-C₁-C₁₂ alkylene, heteroaryl-C₁-C₁₂ alkylene-C(O)—, or —C(O)NR⁴⁶R⁴⁷, wherein one of R⁴⁶ and R⁴⁷ is H or C₁-C₁₂ alkylene and the other is C₁-C₁₂ alkylene; R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, provided that R⁴⁴ and R⁴⁵ are not both H; each R⁴⁶ is independently selected from —NR⁵⁰—, —NR⁵⁰—C₁-C₆alkylene-NR⁵⁰—, —NR⁵⁰—C(O)—NR⁵⁰—S(O)₂—NR⁵⁰— or —NR⁵⁰—C(O)—C₁₋₆alkylene-; each R⁵⁰ is independently selected from H, C₁-C₆ alkyl, or polyhydroxyl group; each n40 is independently 2 to 26; n41 is 1 to 6; and n42 is 1 to 6;

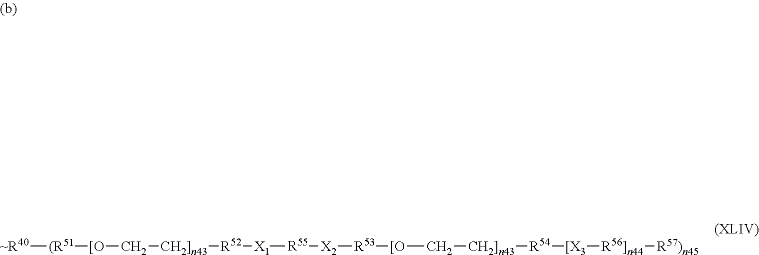

or a stereoisomer or salt thereof, wherein: R⁴⁰ is an attachment group to site R^{b}, or to the enzyme-cleavable group; R⁵¹, R⁵², R⁵³ and R⁵⁴ are each, independently, a bond, or C₁-C₆ alkylene; X₁, X₂ and X₃ are each independently —NR^{N}—C(O)— or —C(O)—NR^{N}—; each R^{N} independently represent H, C₁-C₆ alkyl, or polyhydroxyl group; R⁵⁵ and R⁵⁶ each independently represent a bivalent polyhydroxyl group; R⁵⁷ is H, OH or C₁-C₆ alkyl; each n43 is independently 0 to 26, with the proviso that at least one n43 is 1 to 26; n44 is 0 to 10; and n45 is 1 or 2; and

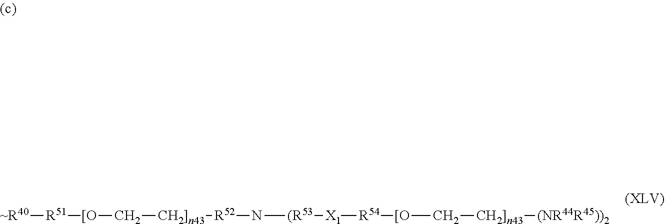

or a stereoisomer or salt thereof, wherein: R⁴⁰ is an attachment group to site R^{b}, or to the enzyme-cleavable group; R⁵¹, R⁵³ and R⁵⁴ are each, independently, a bond or optionally-substituted C₁-C₆ alkylene; R⁵² is a bond, C₁-C₆ alkylene, —C(O)— or —O—C(O)—; each X₁ is independently —NR^{N}—C(O)— or —C(O)—NR^{N}—; each R^{N} independently represent H, C₁-C₆ alkyl, or polyhydroxyl group; R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, provided that R⁴⁴ and R⁴⁵ are not both H; and each n43 is independently 2 to 26.

In some embodiments, provided are Linker compounds comprising a Polar group having one of the following structures prior to attachment to the enzyme-cleavable group and/or to the Linker Unit:

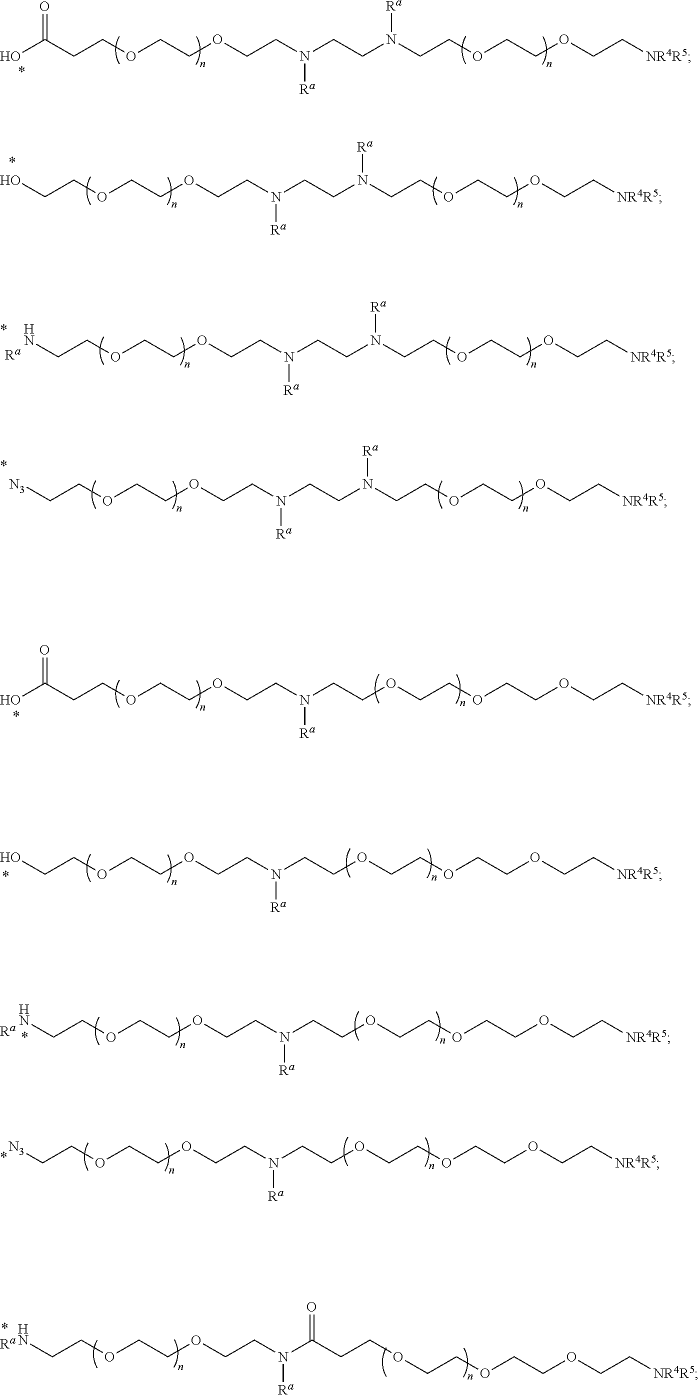

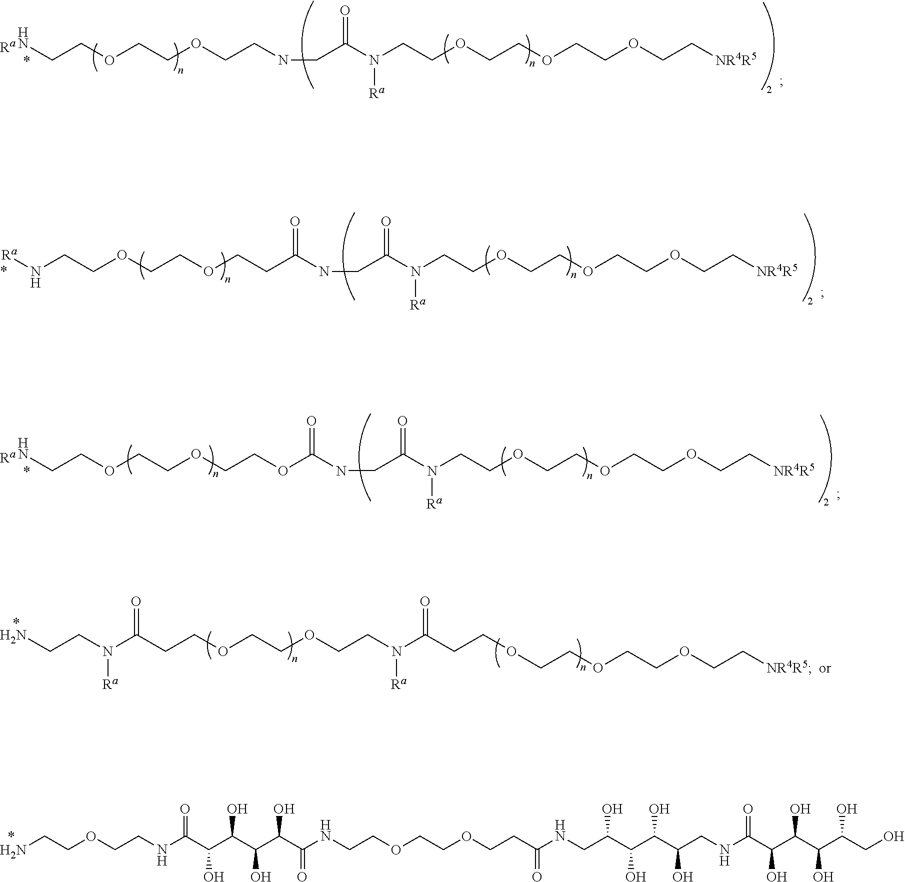

wherein: (*) indicates the attachment site to R^{b}, or to the enzyme-cleavable group; each R is independently H, alkyl or polyhydroxyl group; R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, provided that R⁴⁴ and R⁴⁵ are not both H; and each n is independently 1 to 12.

In some embodiments, provided are Linker compounds comprising a Polar group having a formula selected from:

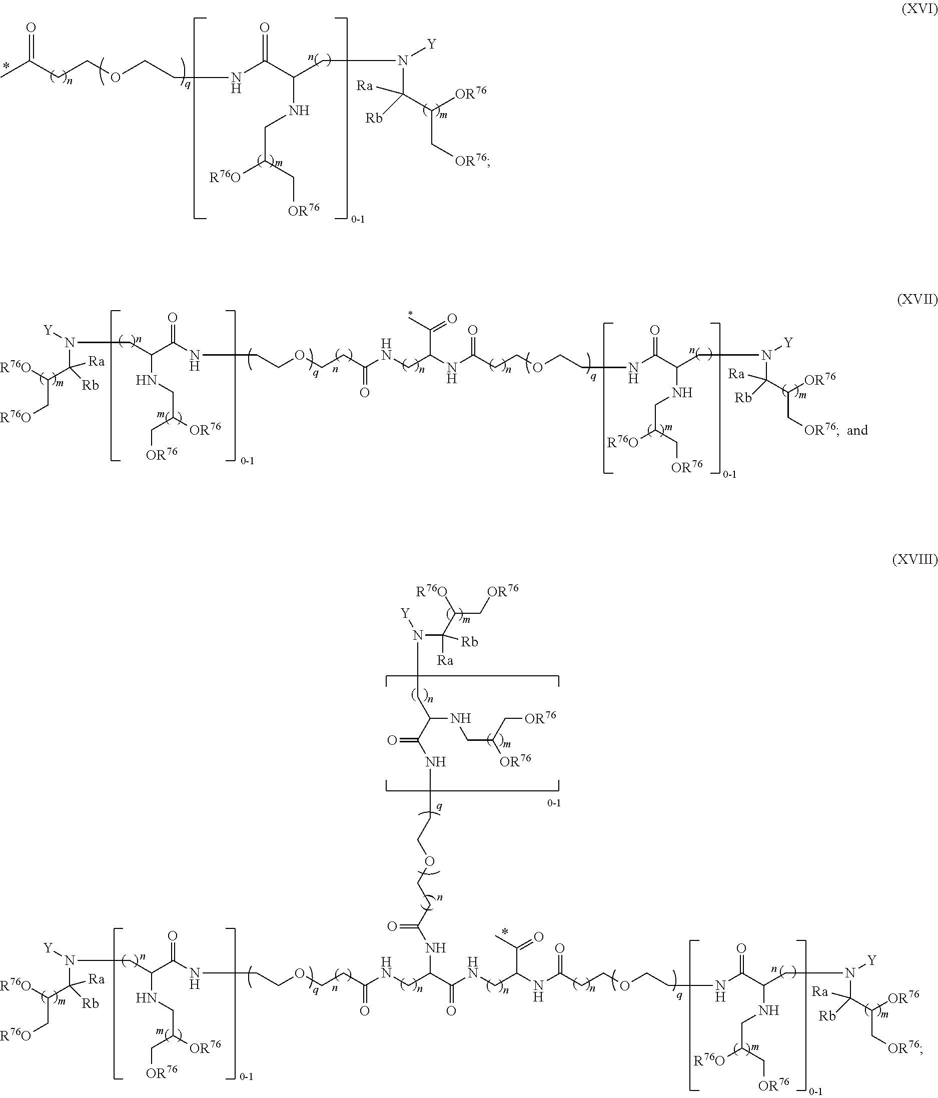

or a stereoisomer or salt thereof, wherein: each Y is independently R⁷⁶ or

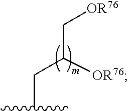

each R⁷⁶ is independently H, acetyl, —P(═O)(OH)—, or —(CH₂)ᵥ—O—S(═O)₂(OH); each R^{α} and R_{b} is independently H or Rₐ and R_{b} are taken together with the carbon to which they are attached to form an oxo group; each q is independently 2-26; each m is independently 1 to 4; each n is independently 1 to 4; each v is independently 1 to 6; and each * is an attachment site to R^{b}, or to the enzyme-cleavable group. In some embodiments, Y is R⁷⁶. In other embodiments, Y is

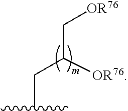

In some embodiments, provided are Linker compounds comprising a Polar group having a formula selected from:

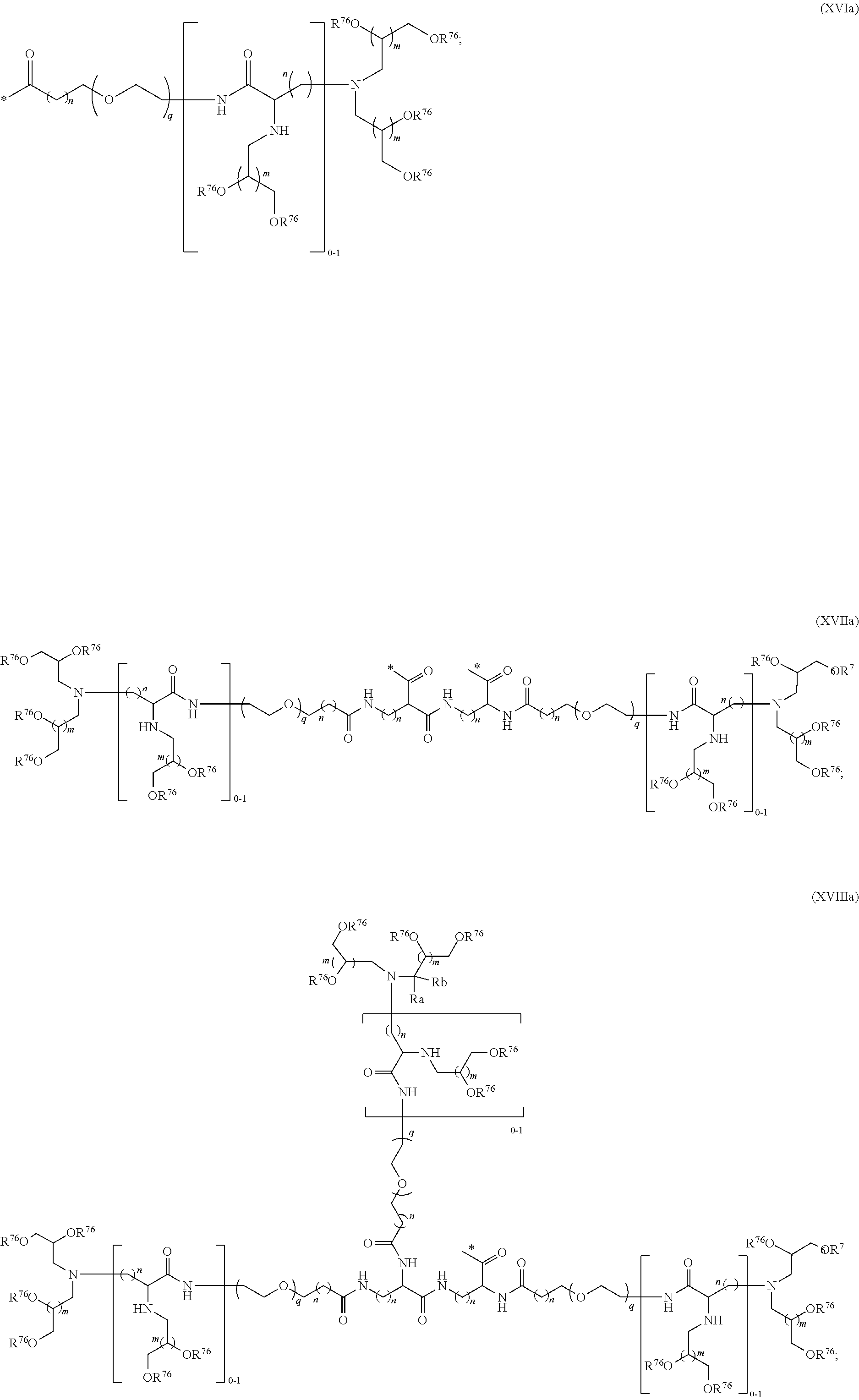

or a stereoisomer or salt thereof, wherein: each R⁷⁶ is independently H, acetyl, —P(═O)(OH)—, or —(CH₂)ᵥS(═O)₂(OH); each q is independently 2-26; each m is independently 1 to 4; each n is independently 1 to 4; each v is independently 1 to 6; and each * is an attachment site to R^{b}, or to the enzyme-cleavable group.

In some embodiments, provided are Linker compounds comprising a Polar group having a formula selected from:

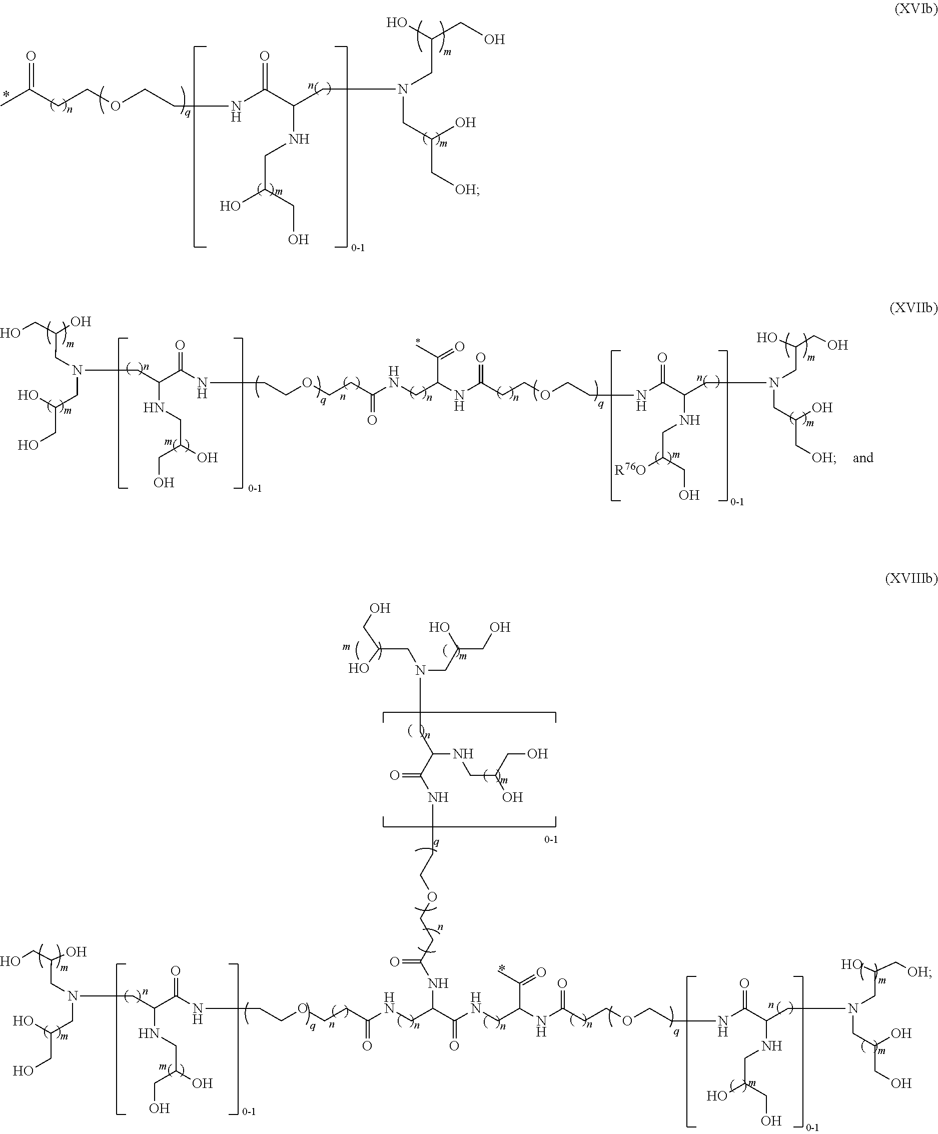

or a stereoisomer or salt thereof, wherein: each q is independently 2-26; each m is independently 1 to 4; each n is independently 1 to 4; and each * is an attachment site to R^{b}, or to the enzyme-cleavable group.

In some embodiments, each Rₐ and R_{b} as described above are independently H. In other embodiments, Rₐ and R_{b} as described above are taken together with the carbon to which they are attached to form an oxo group.

In some embodiments, q as described above is 10-20. For example, in some embodiments q as described above is 12.

In some embodiments, provided are Linker compounds comprising a Polar group selected from the following, or a stereoisomer or salt thereof:

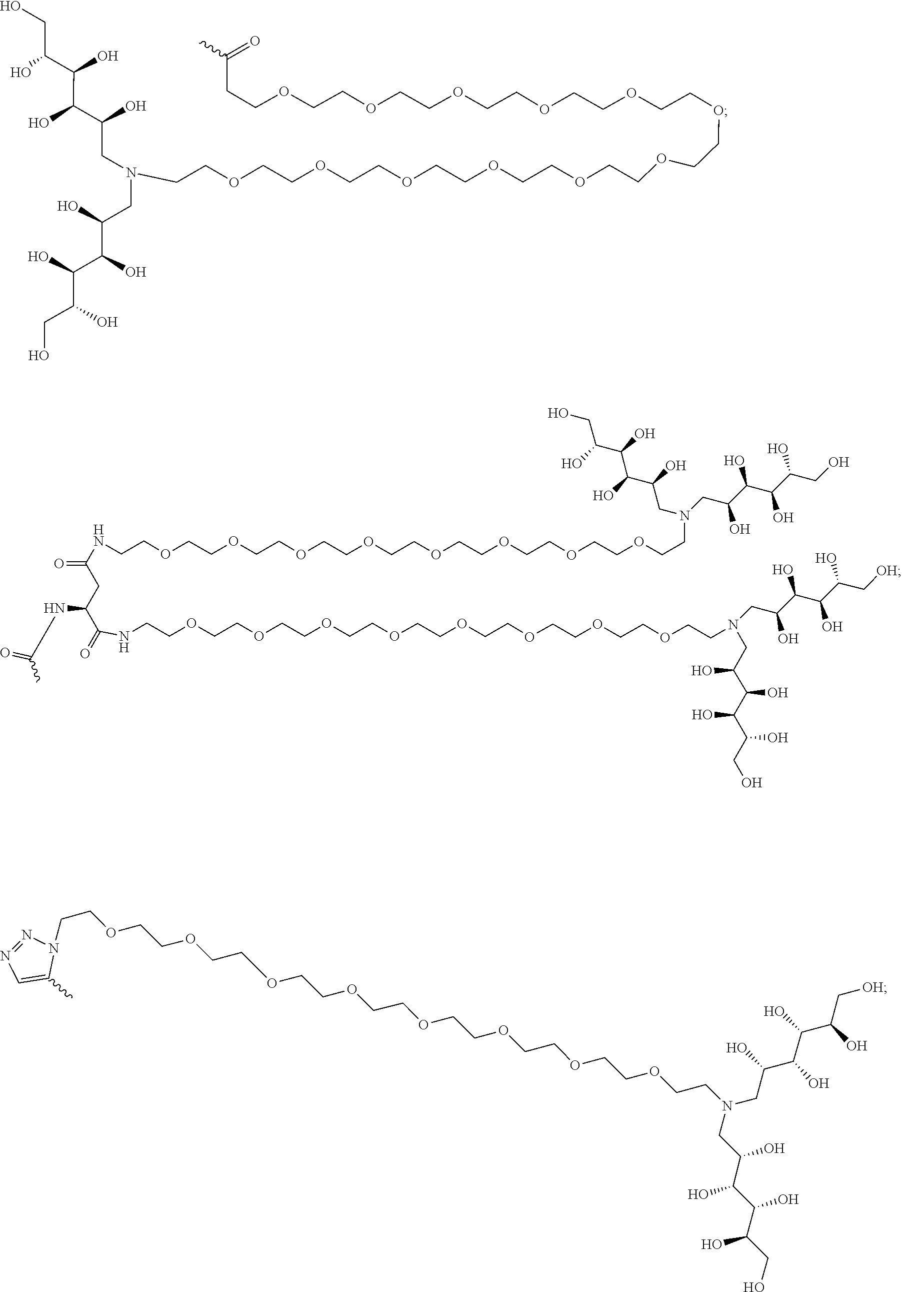

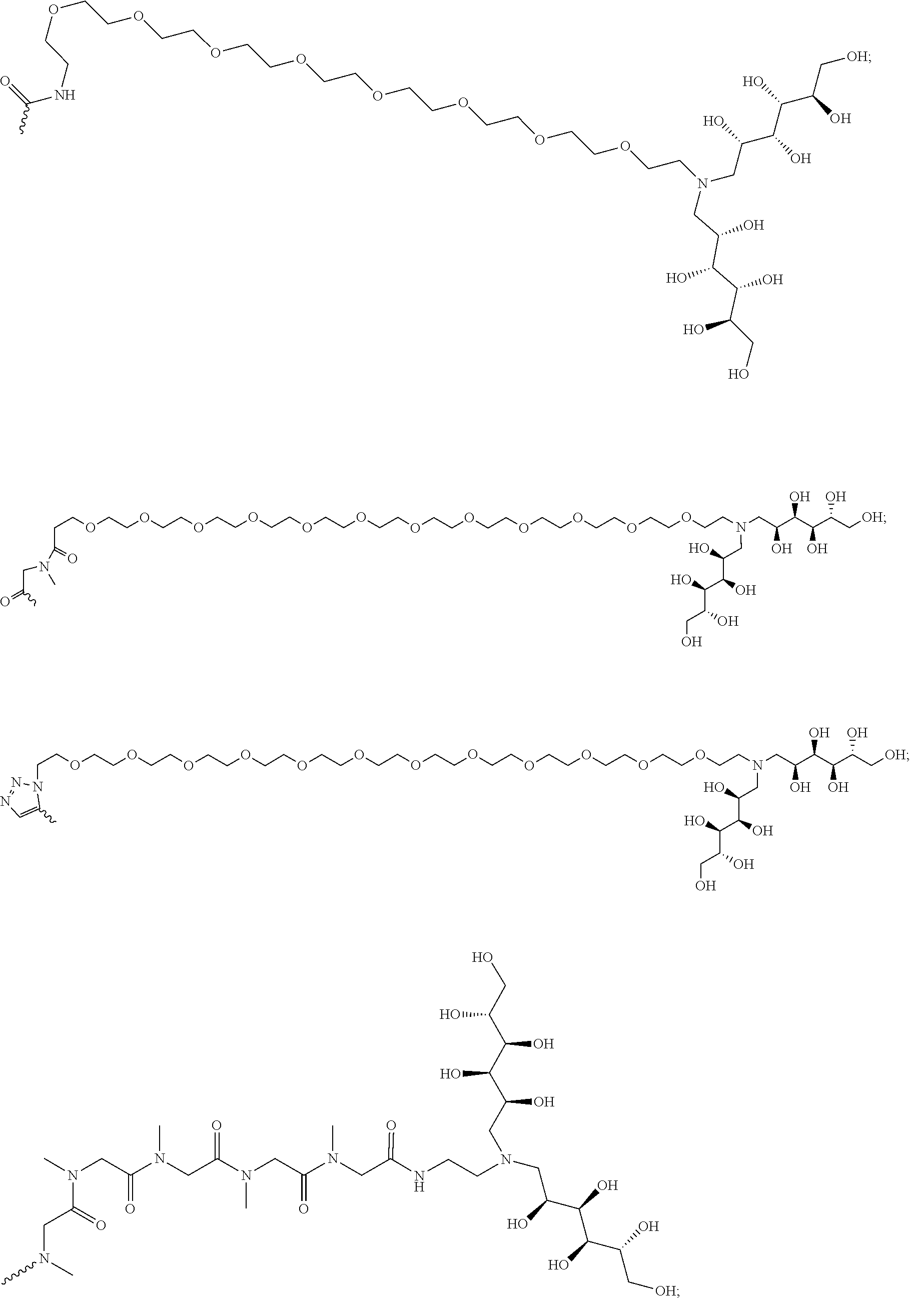

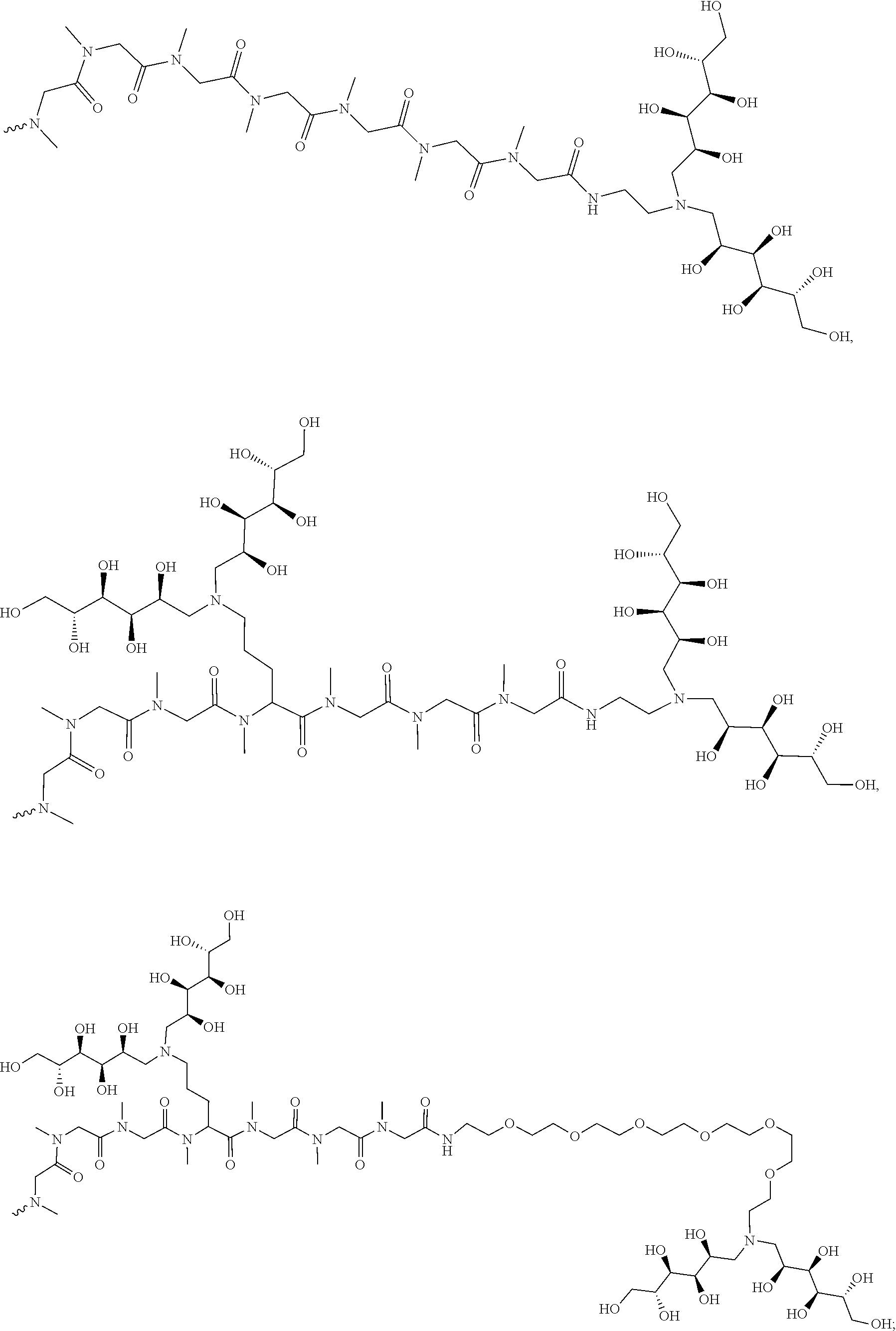

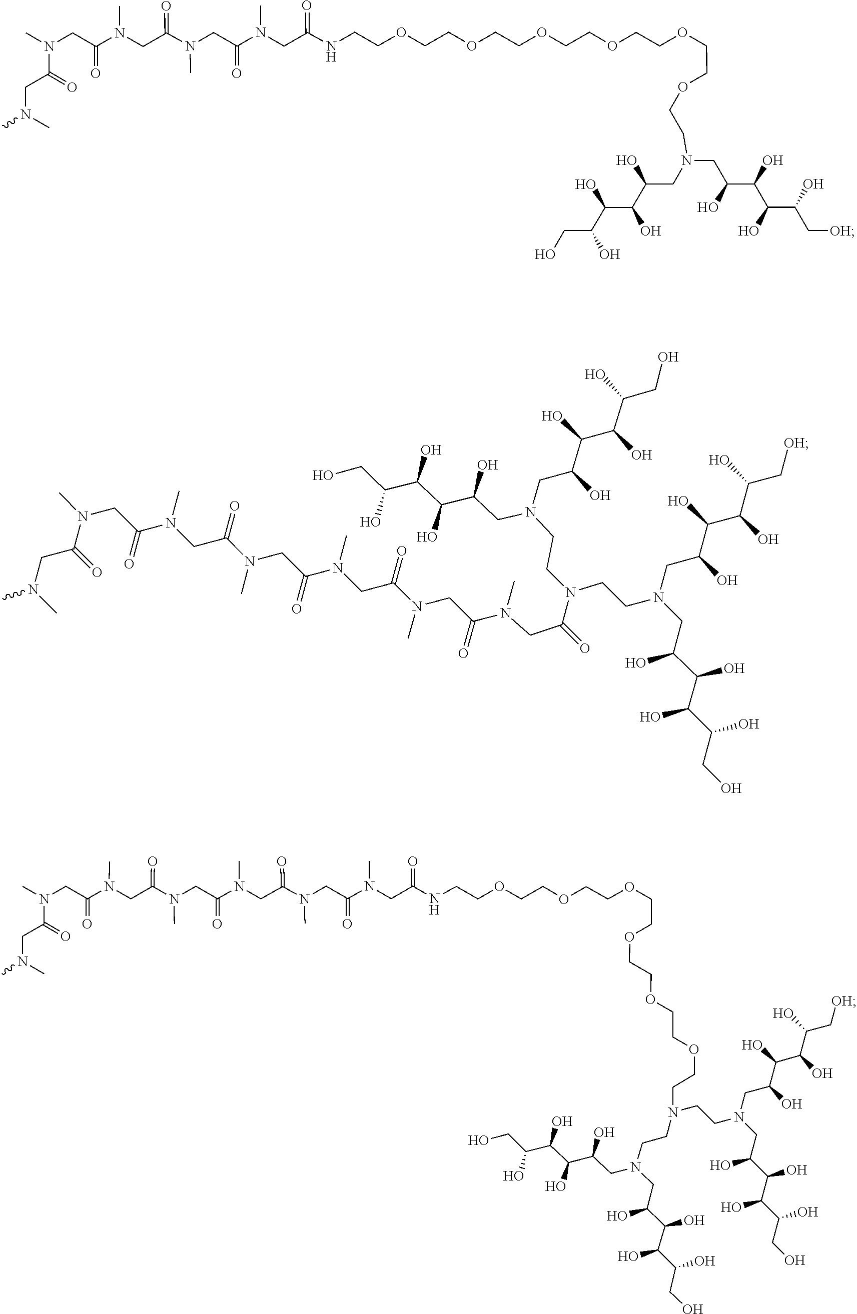

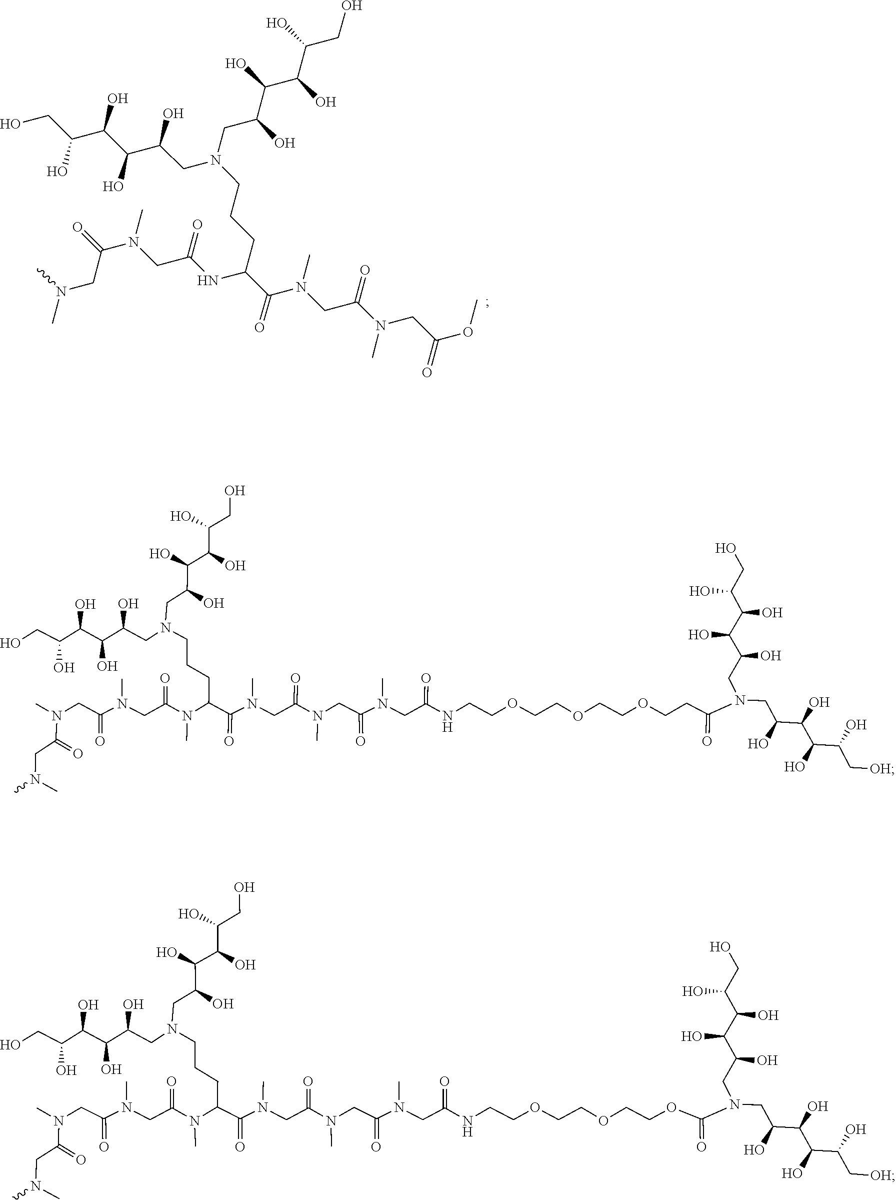

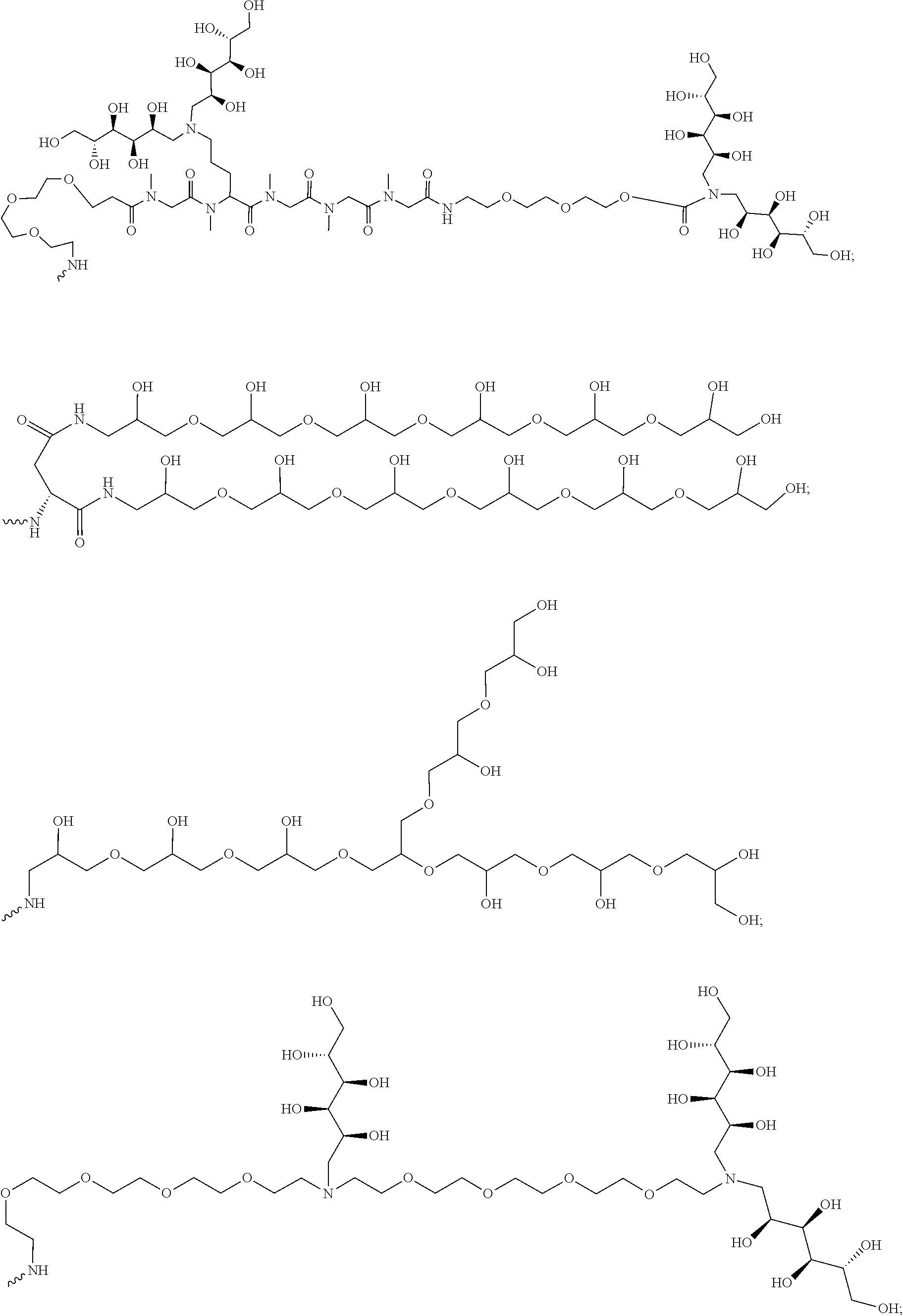

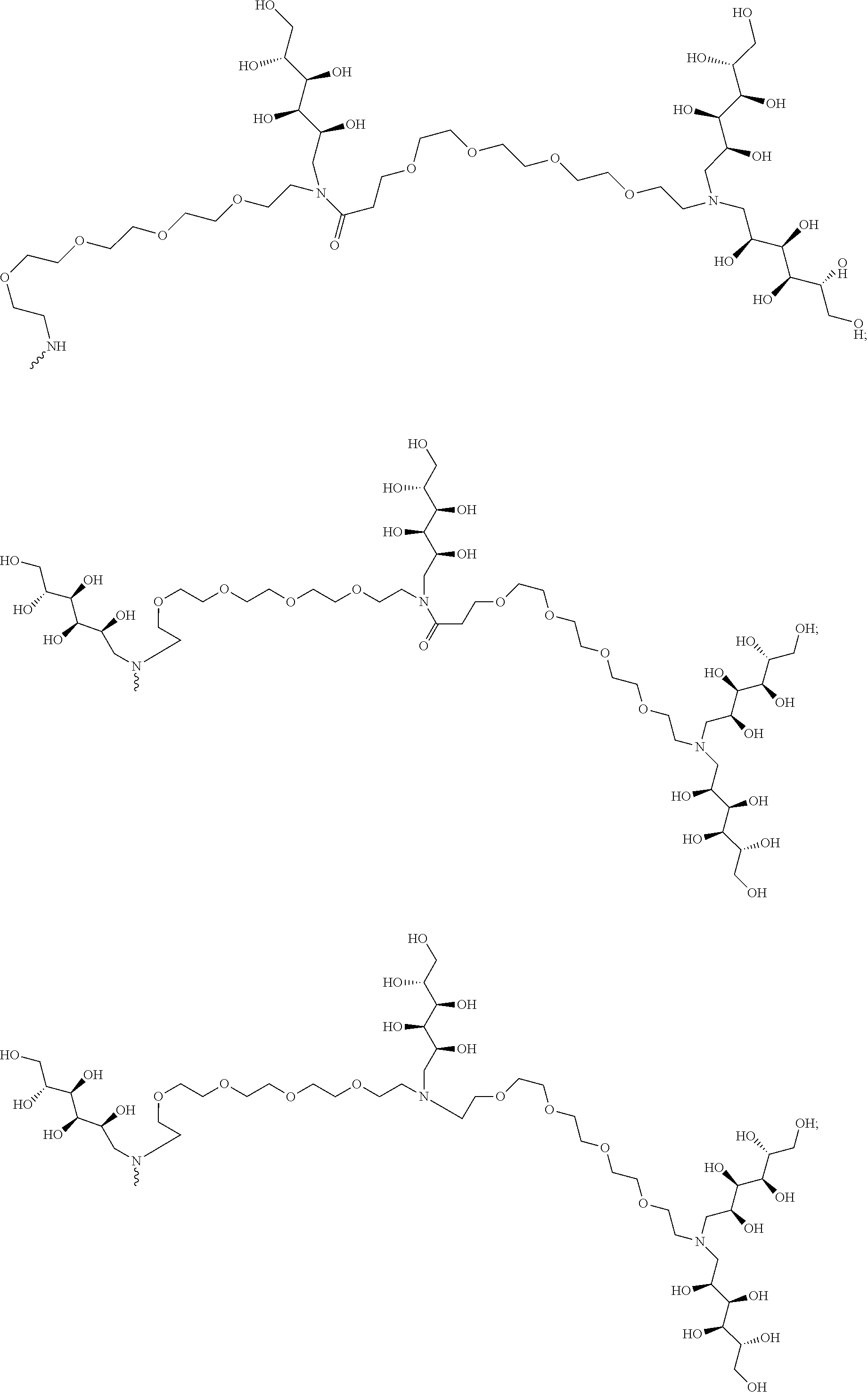

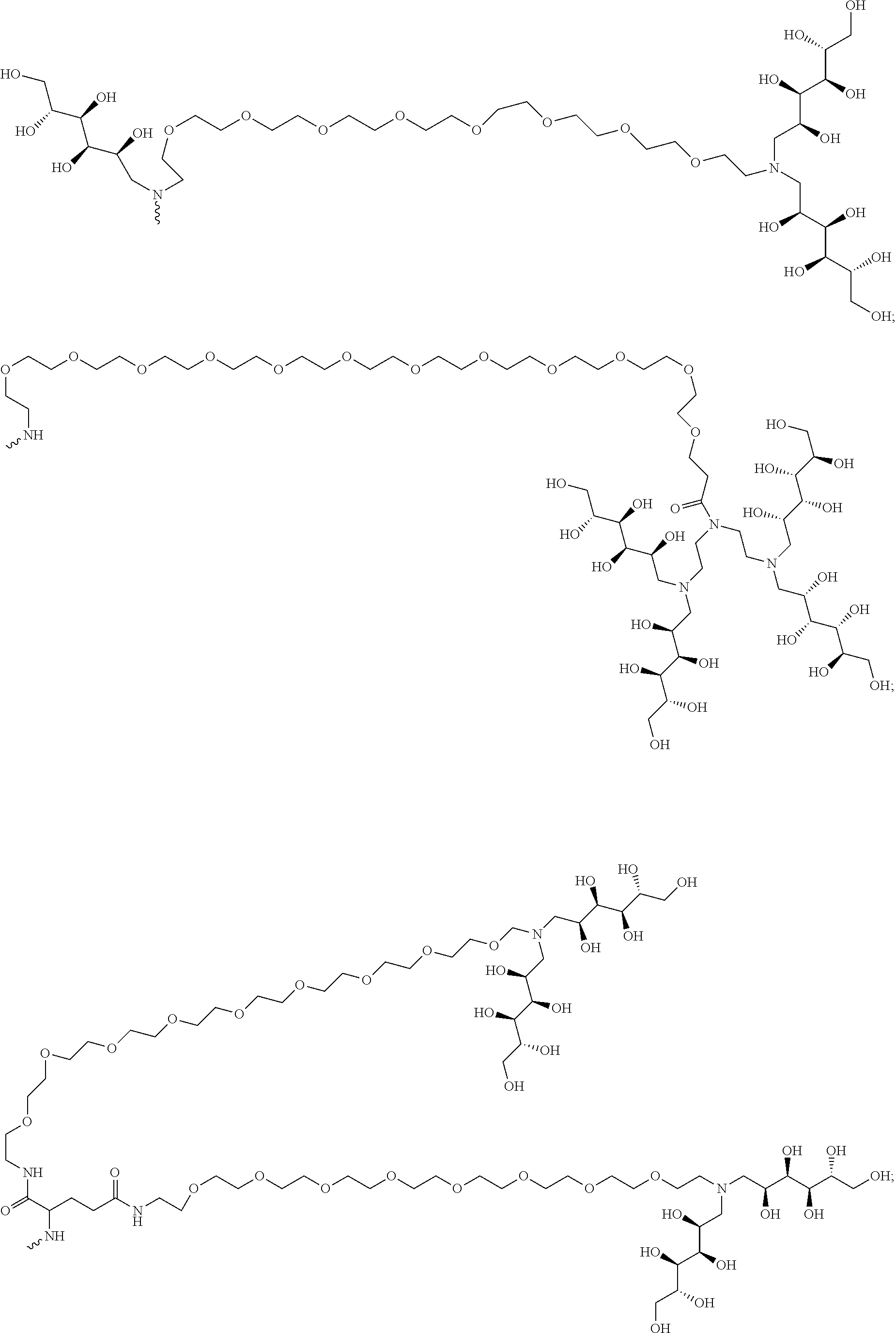

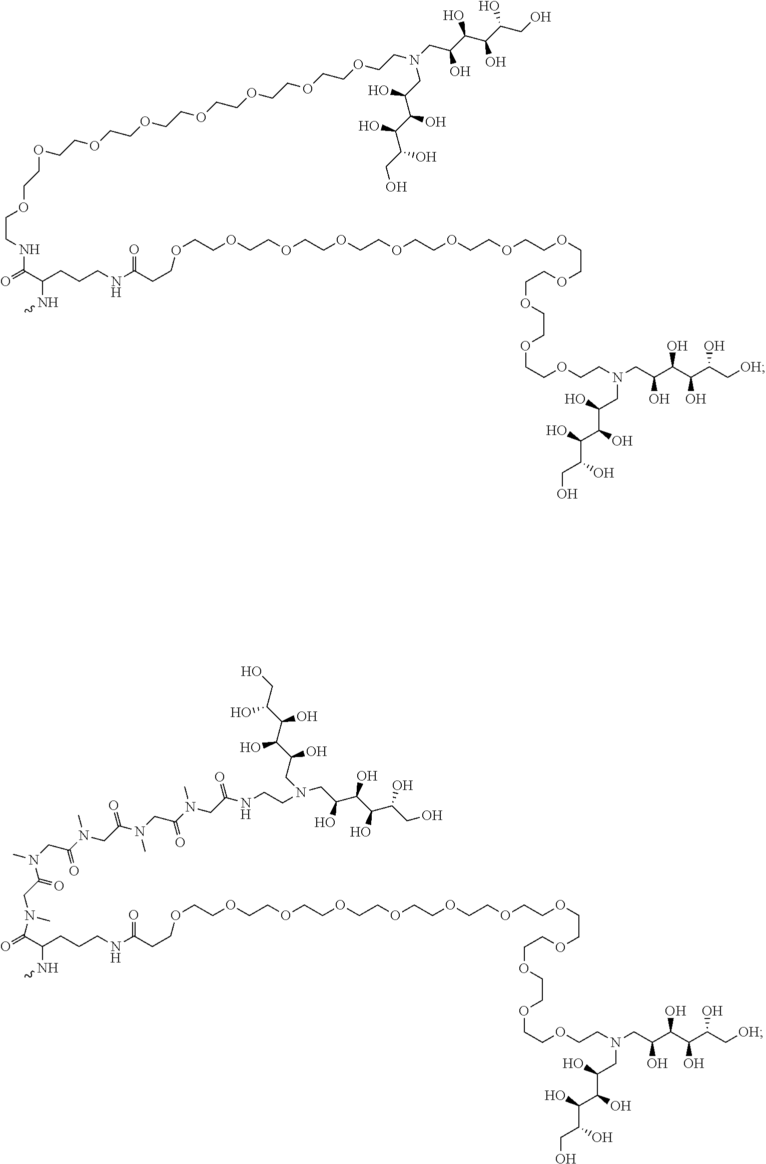

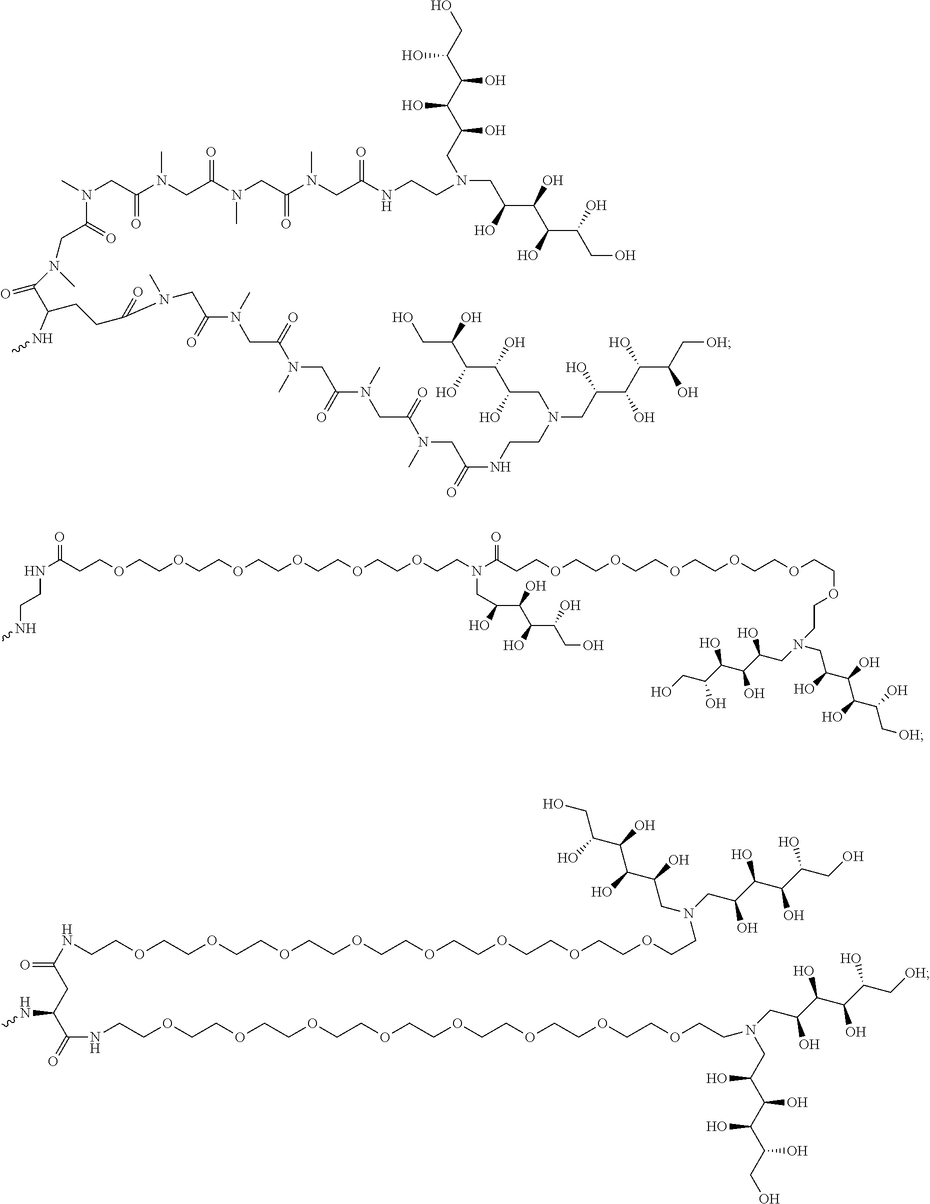

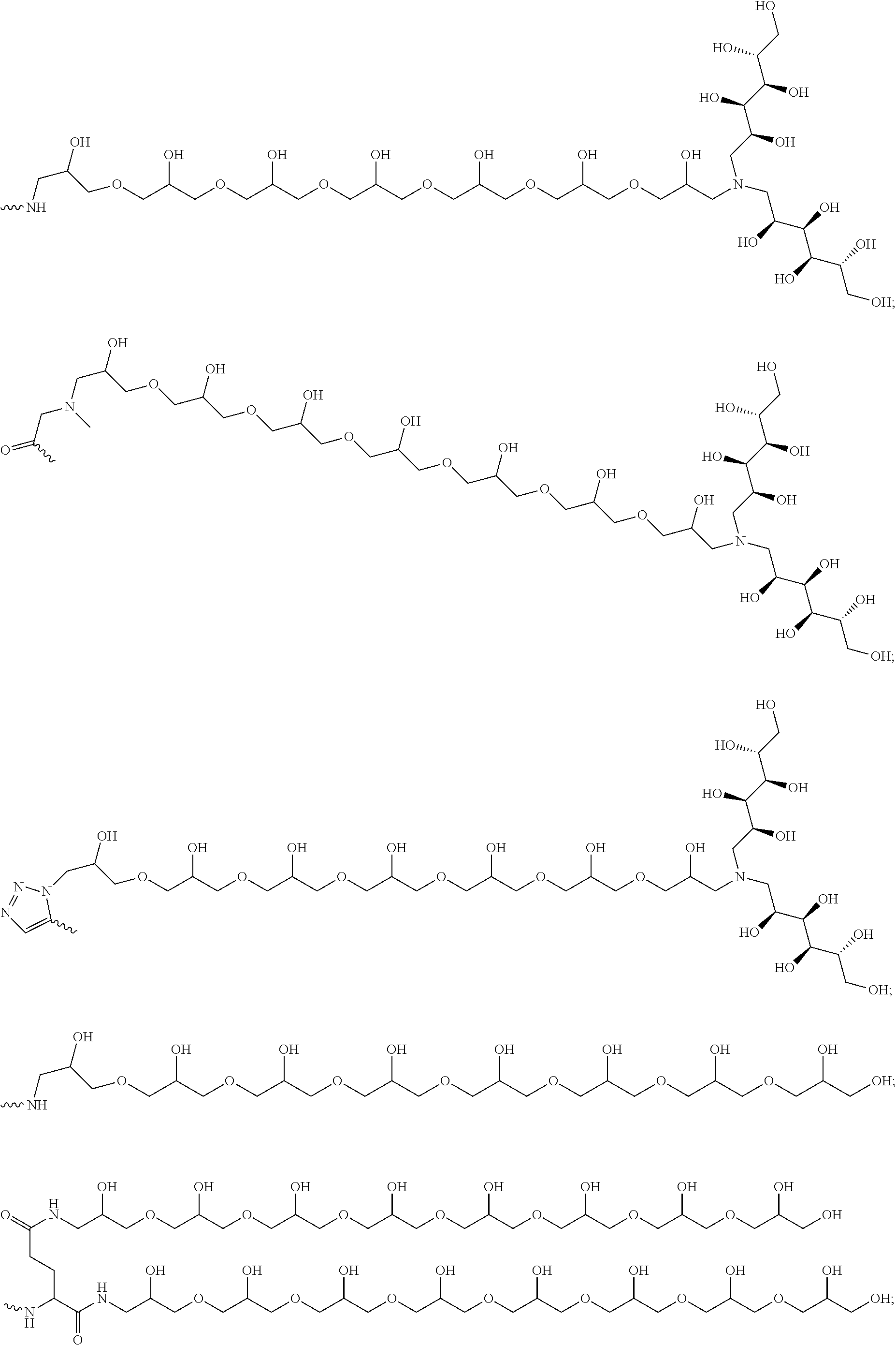

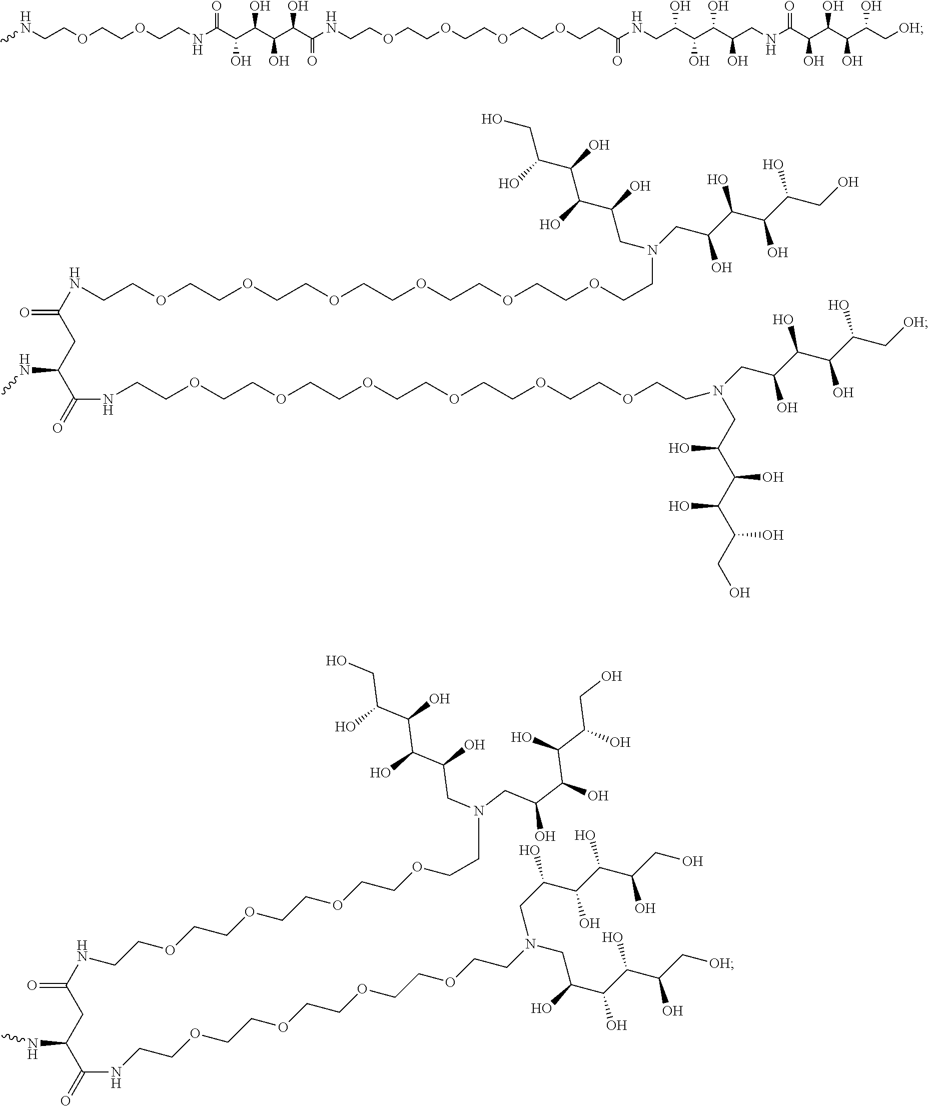

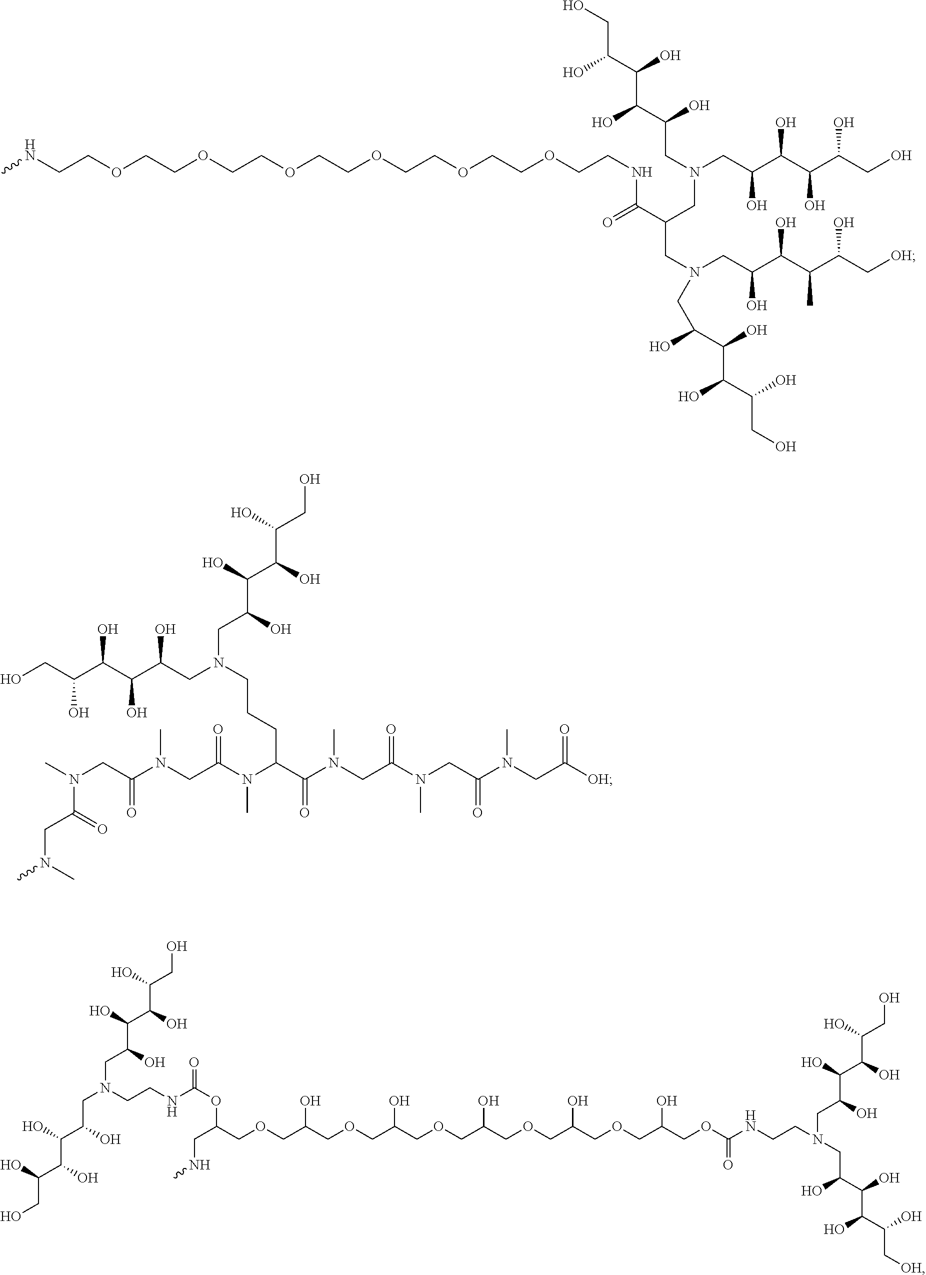

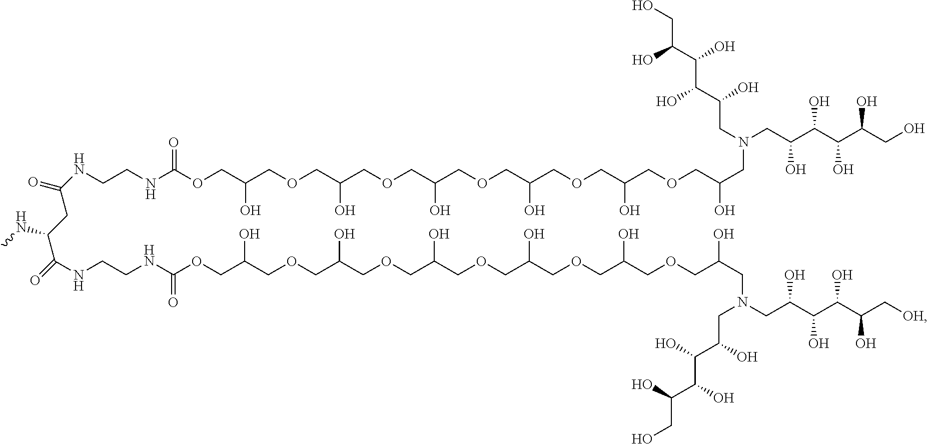

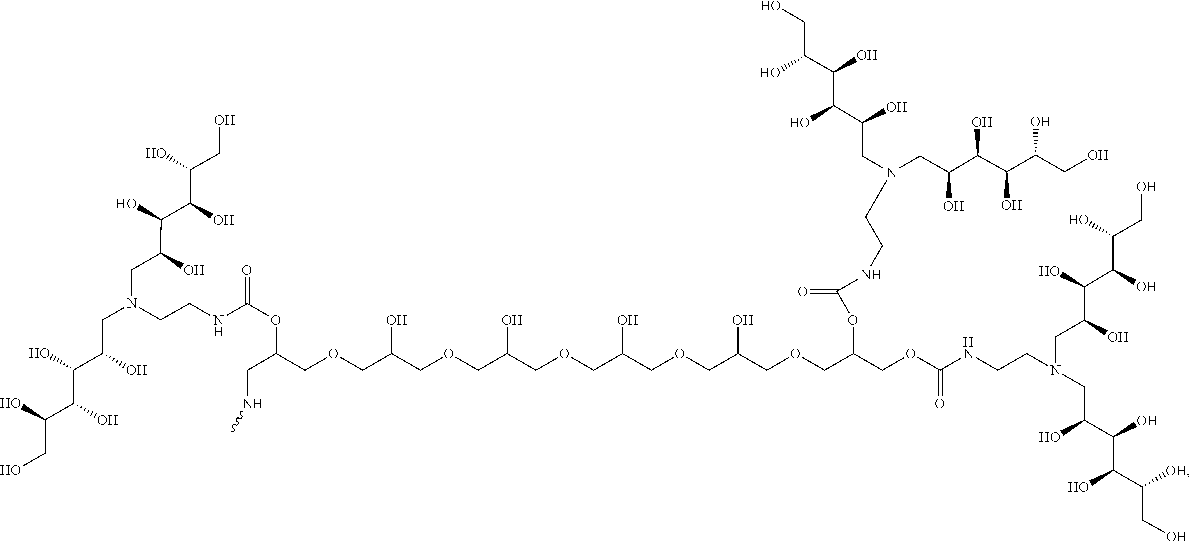

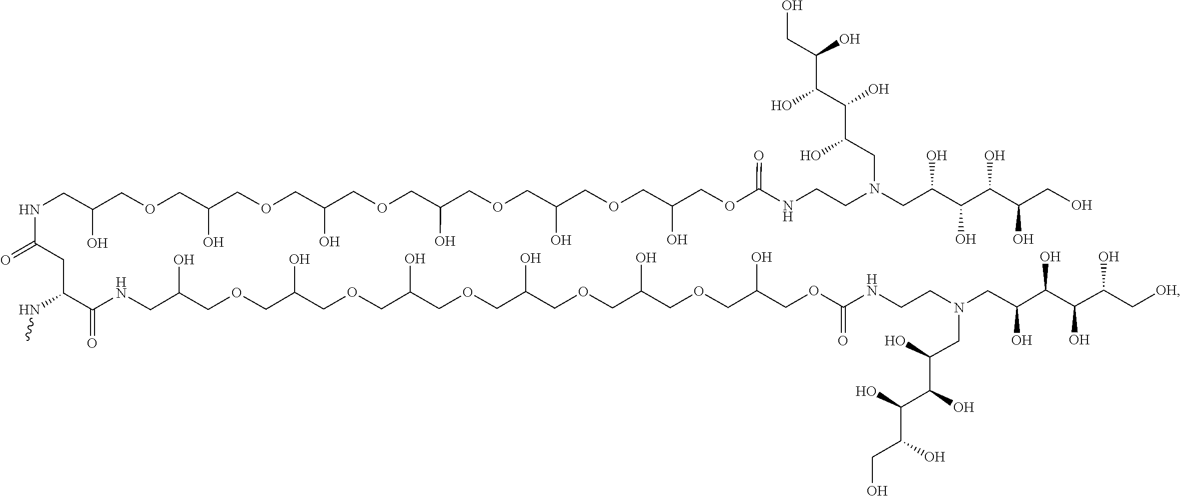

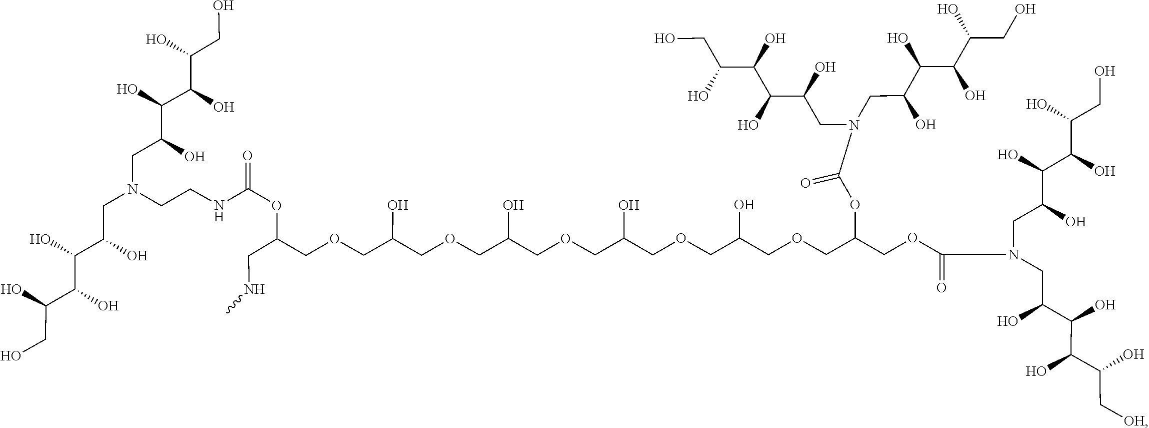

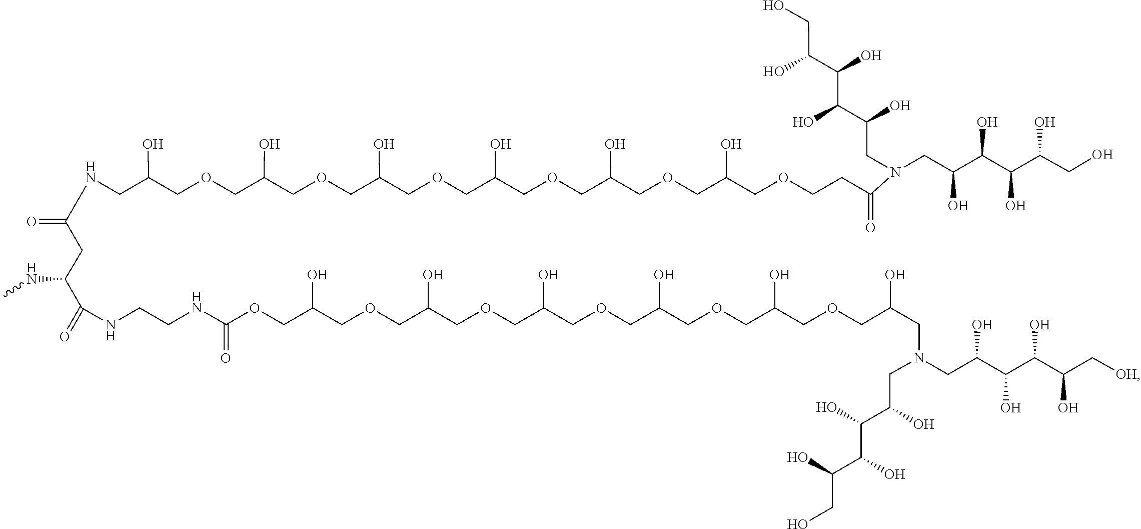

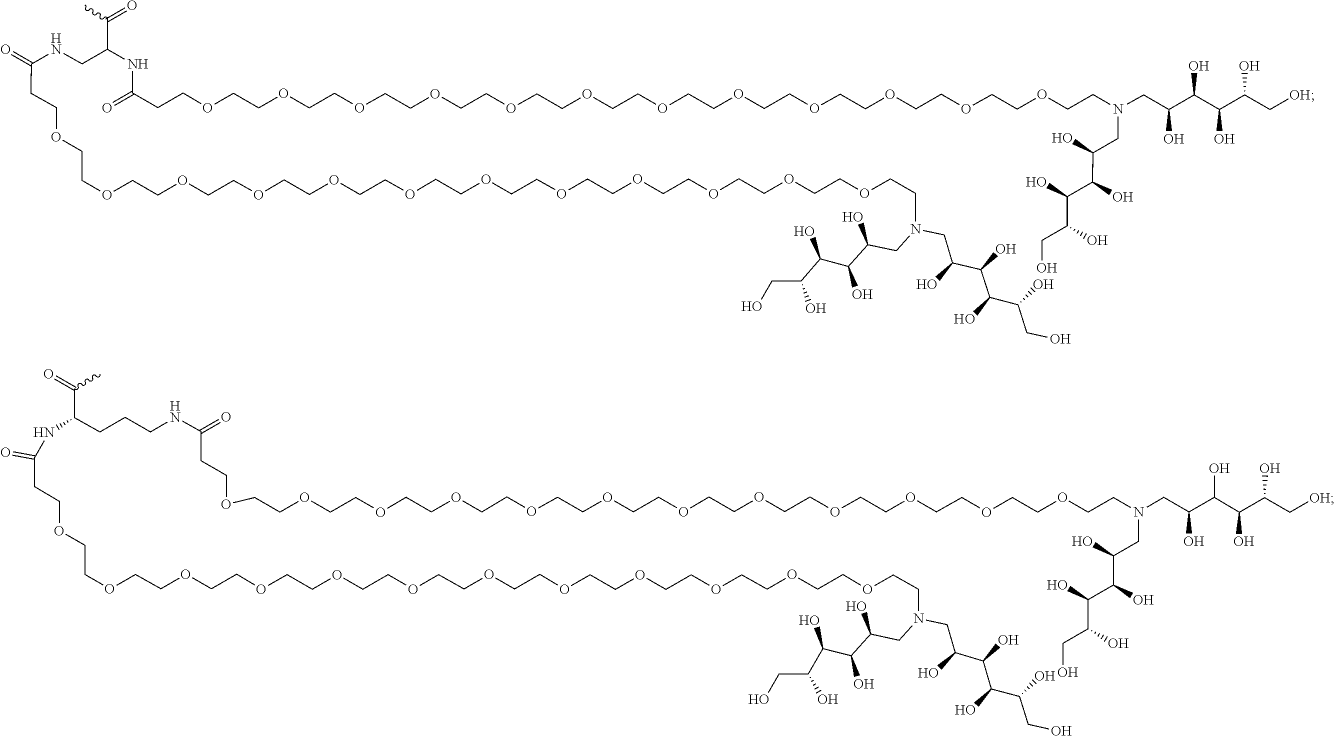

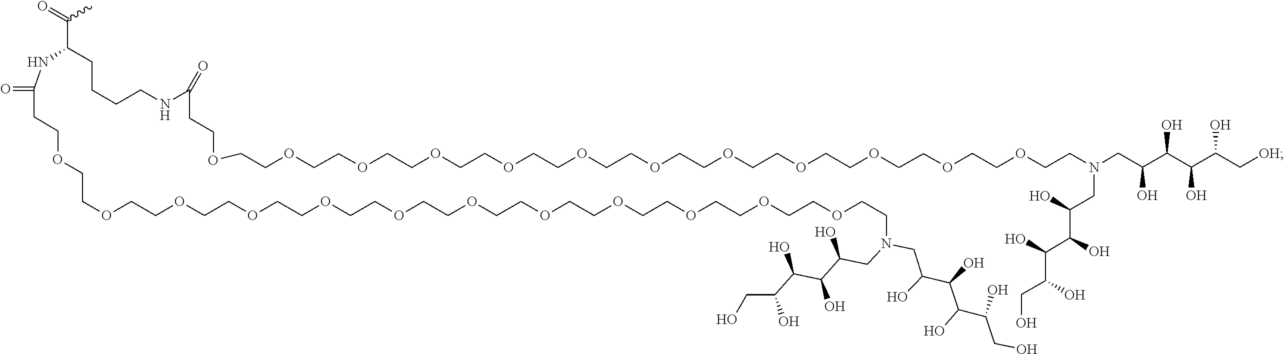

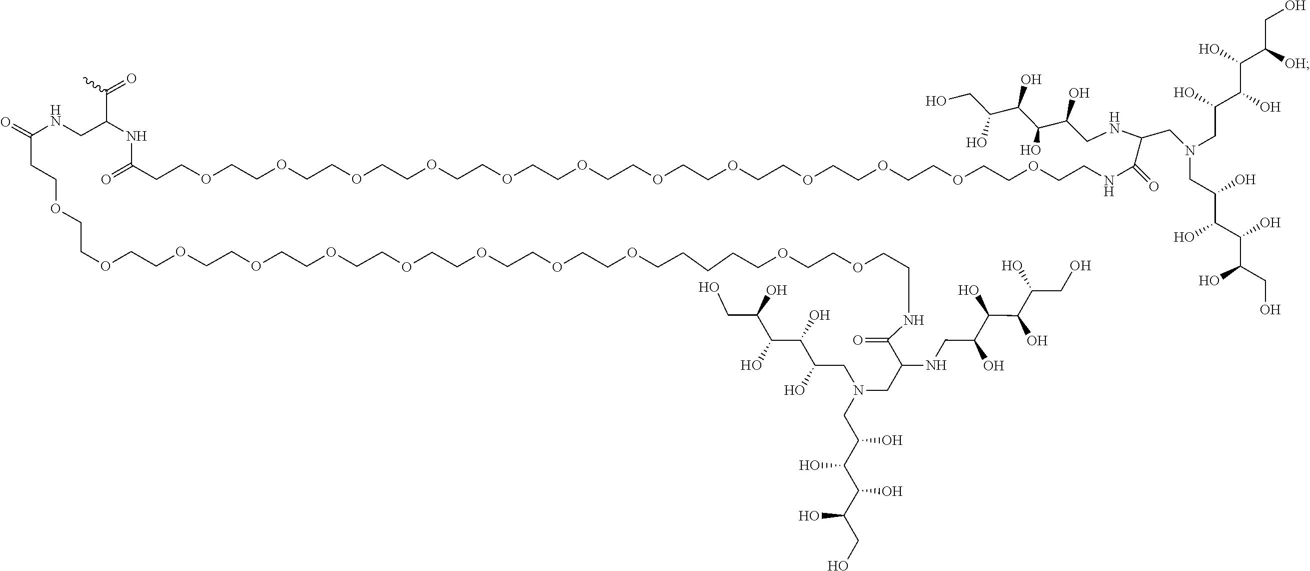

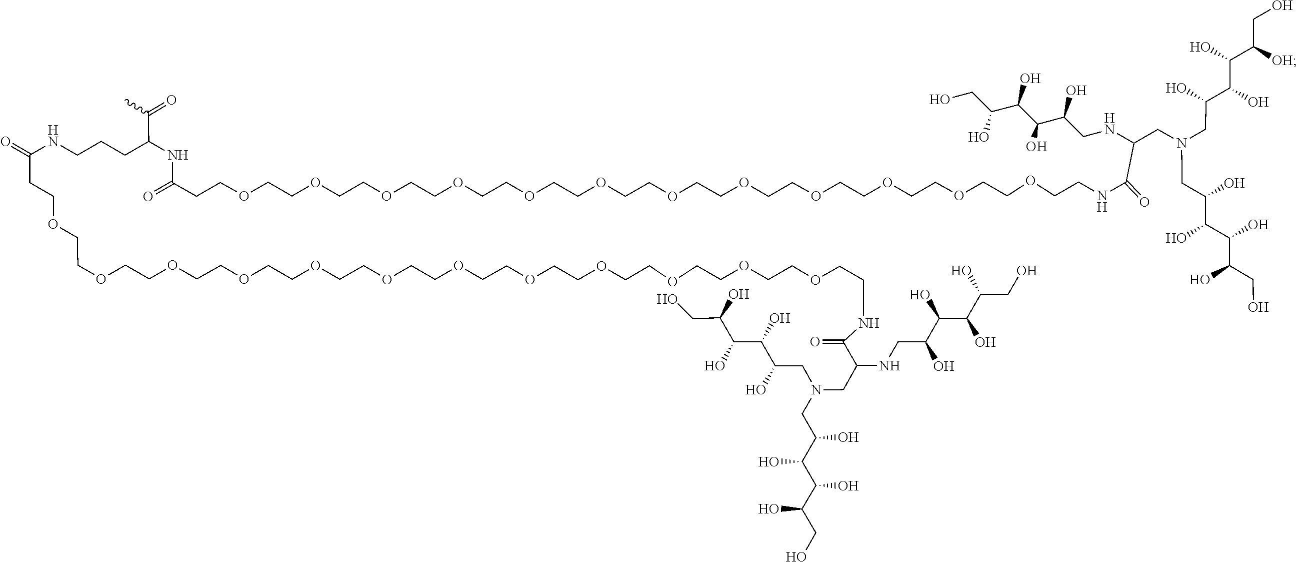

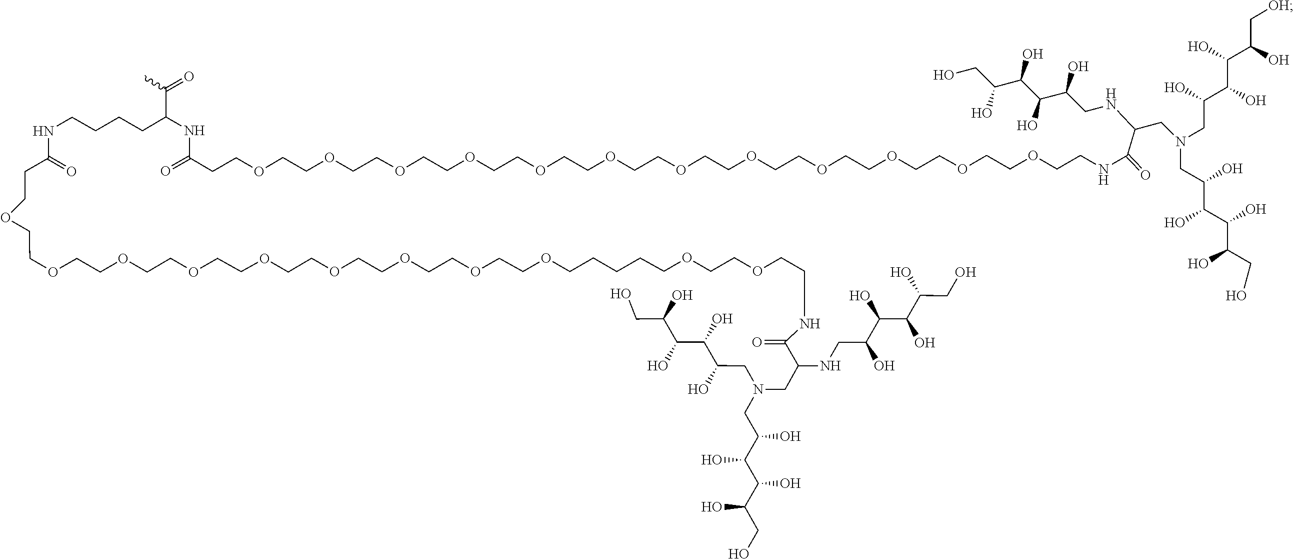

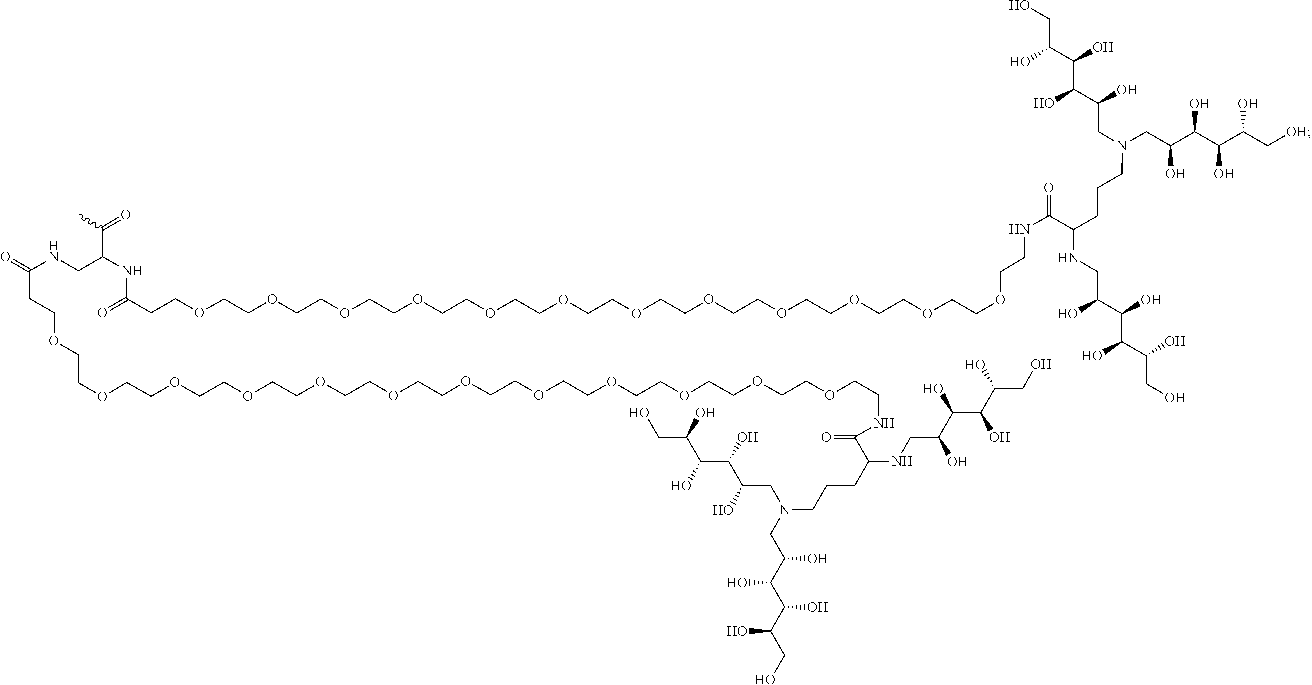

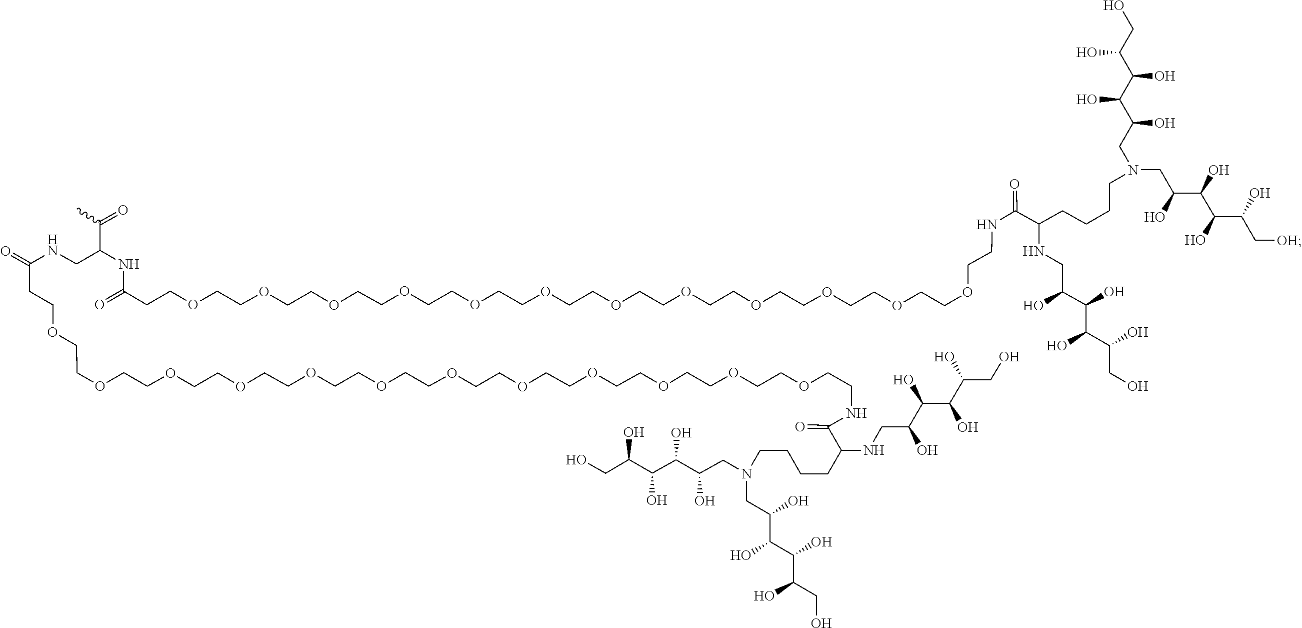

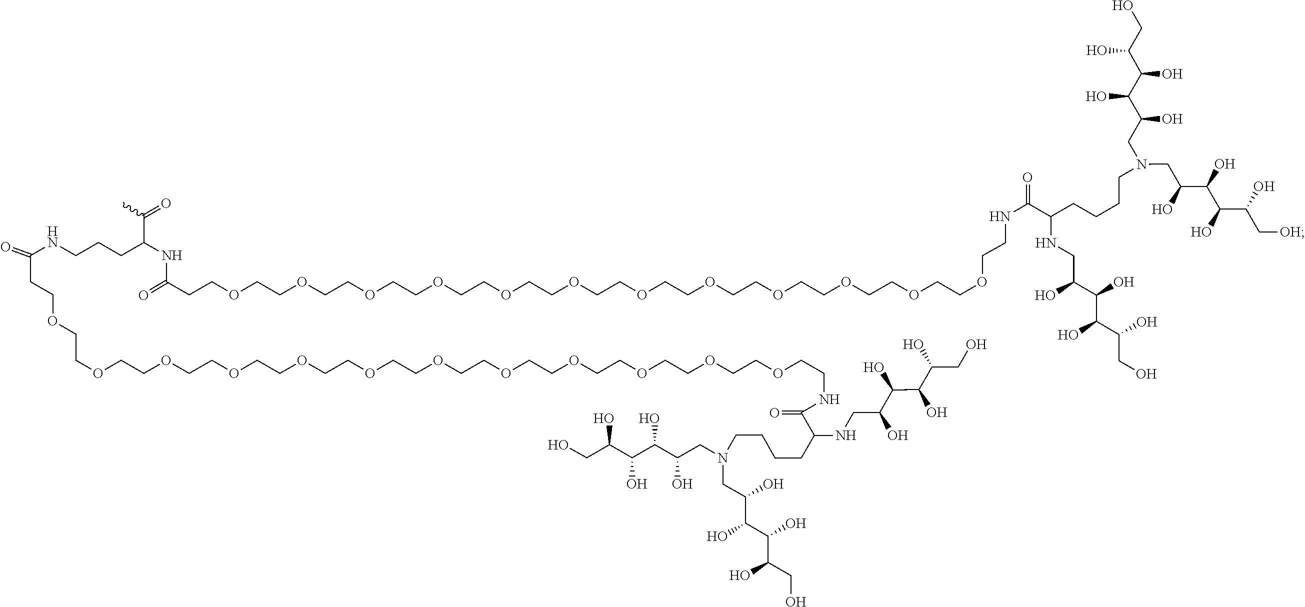

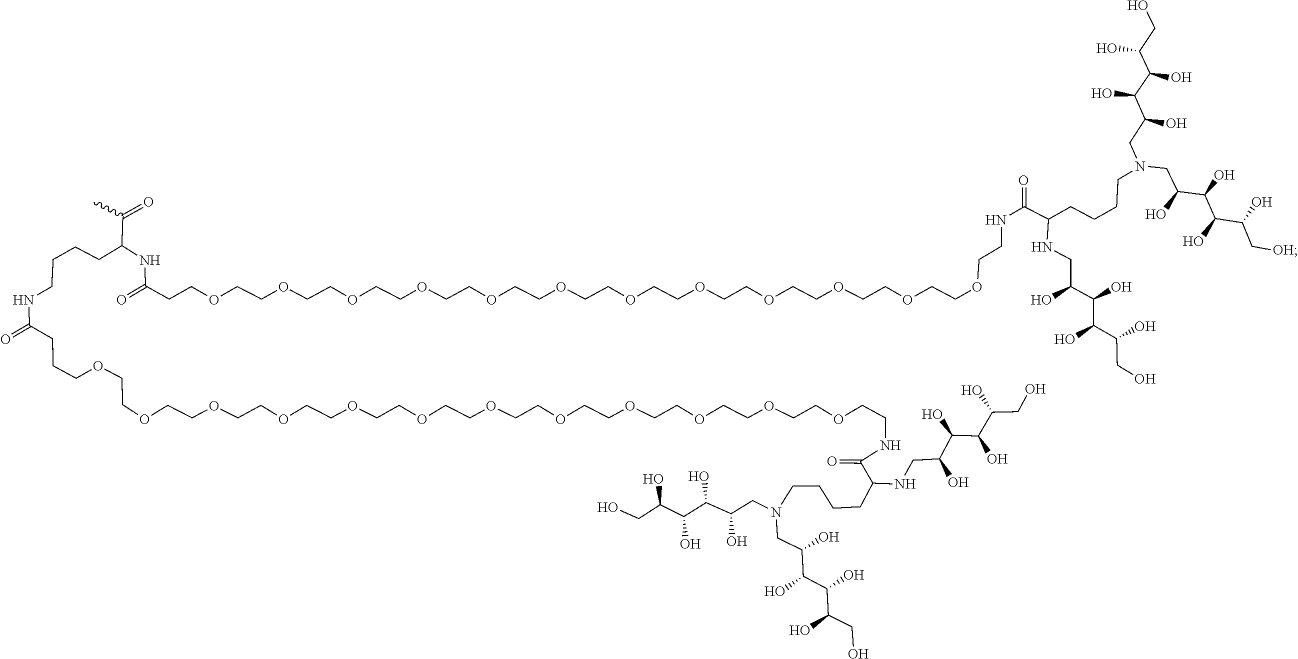

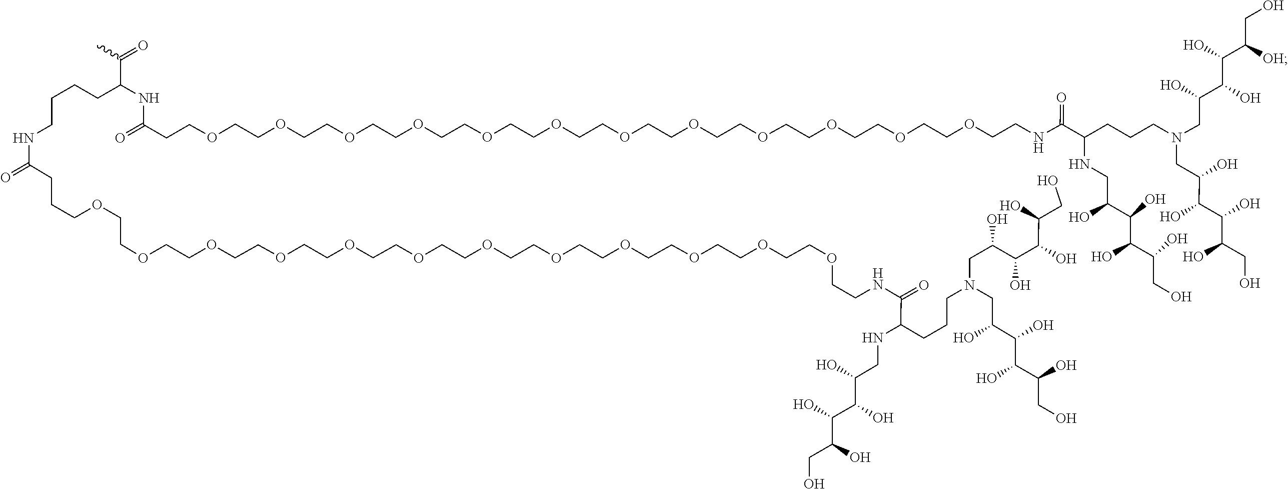

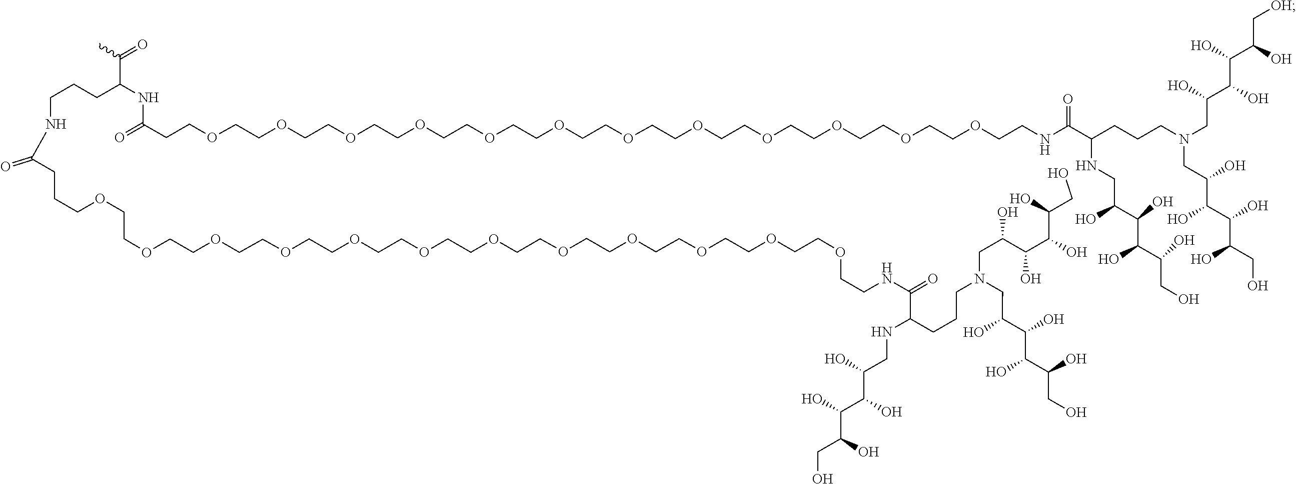

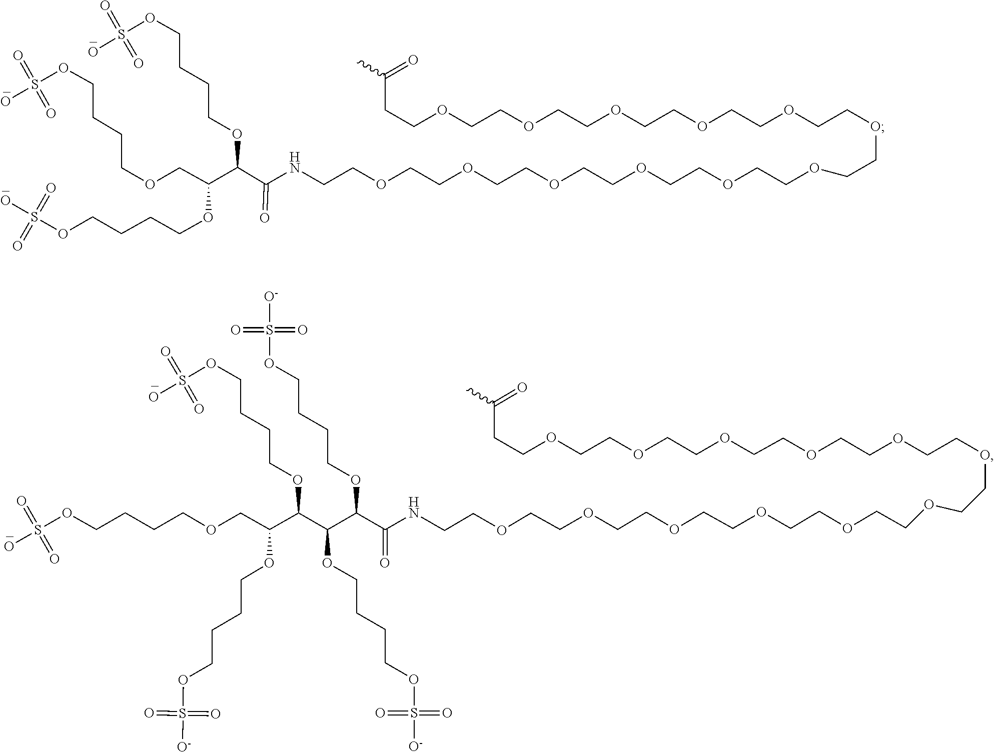

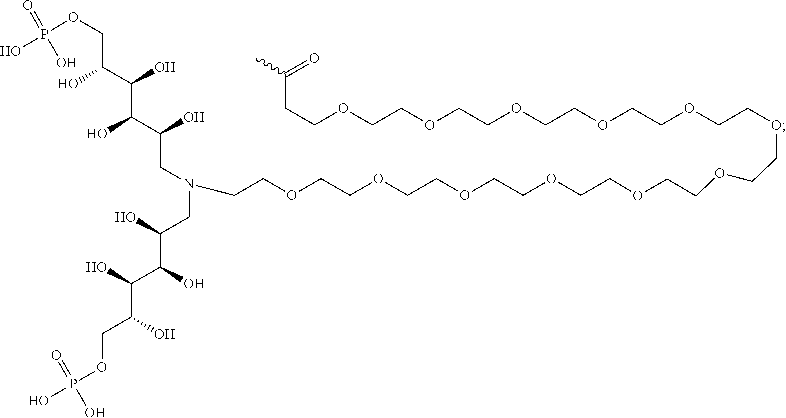

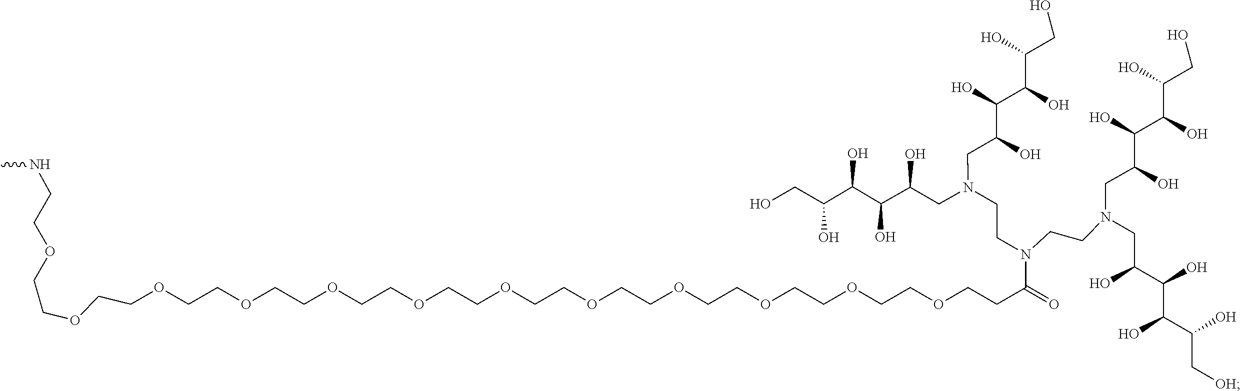

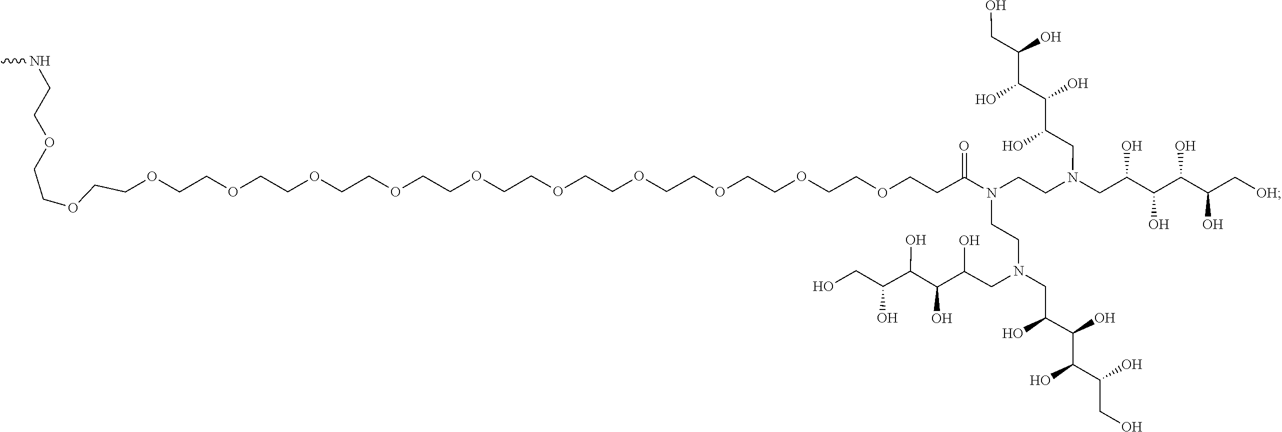

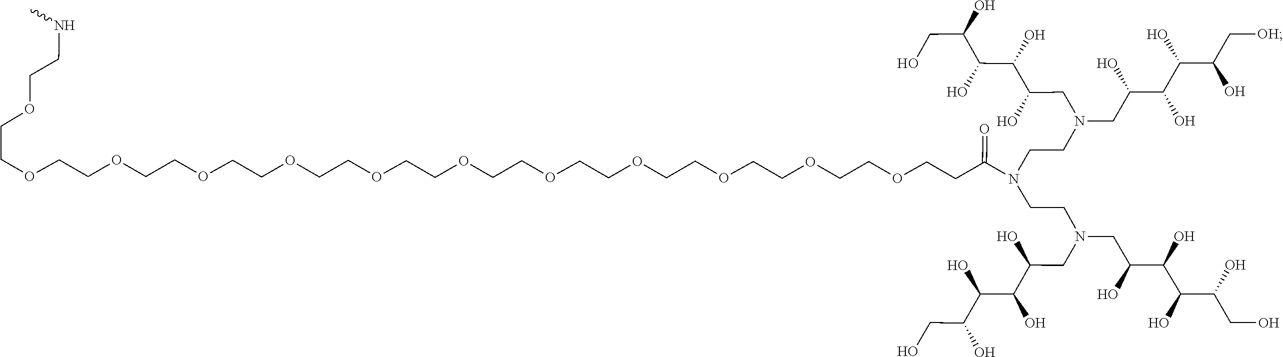

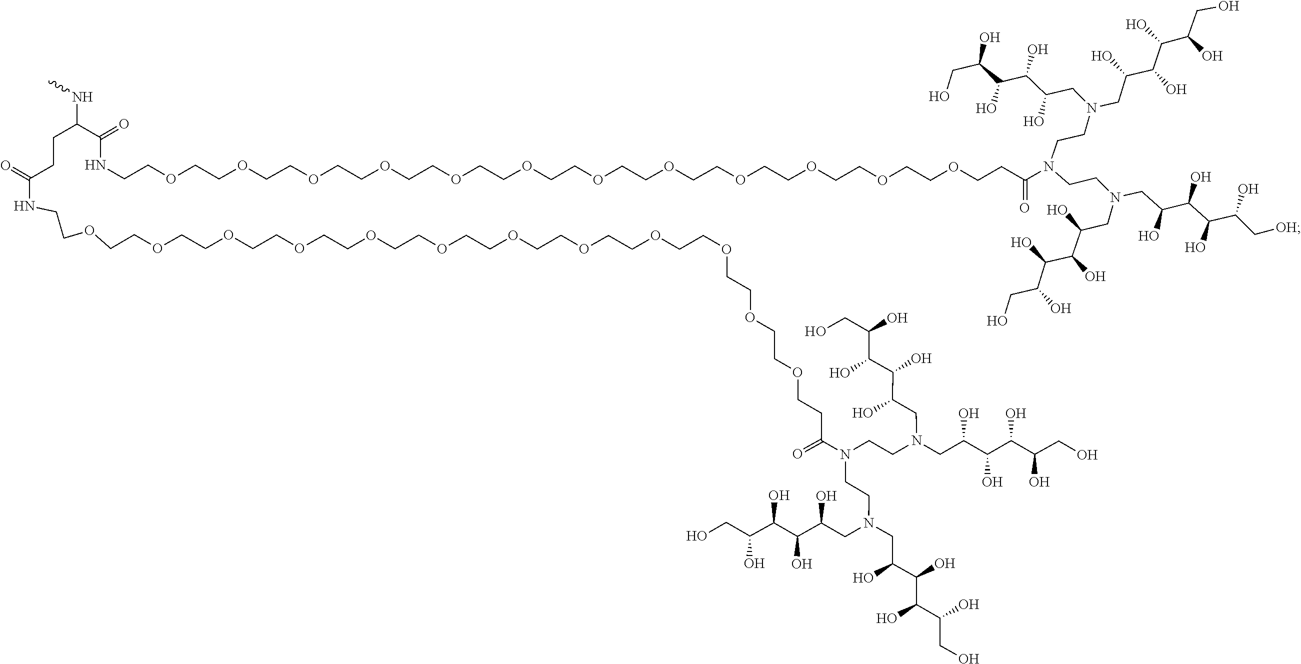

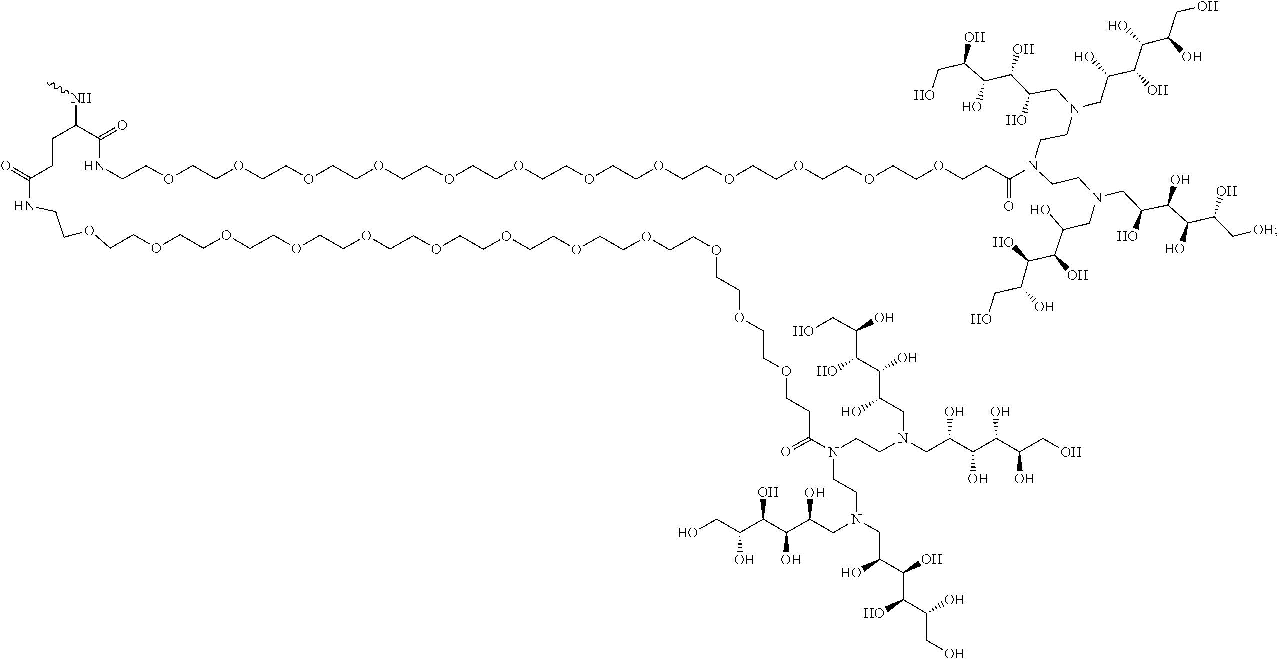

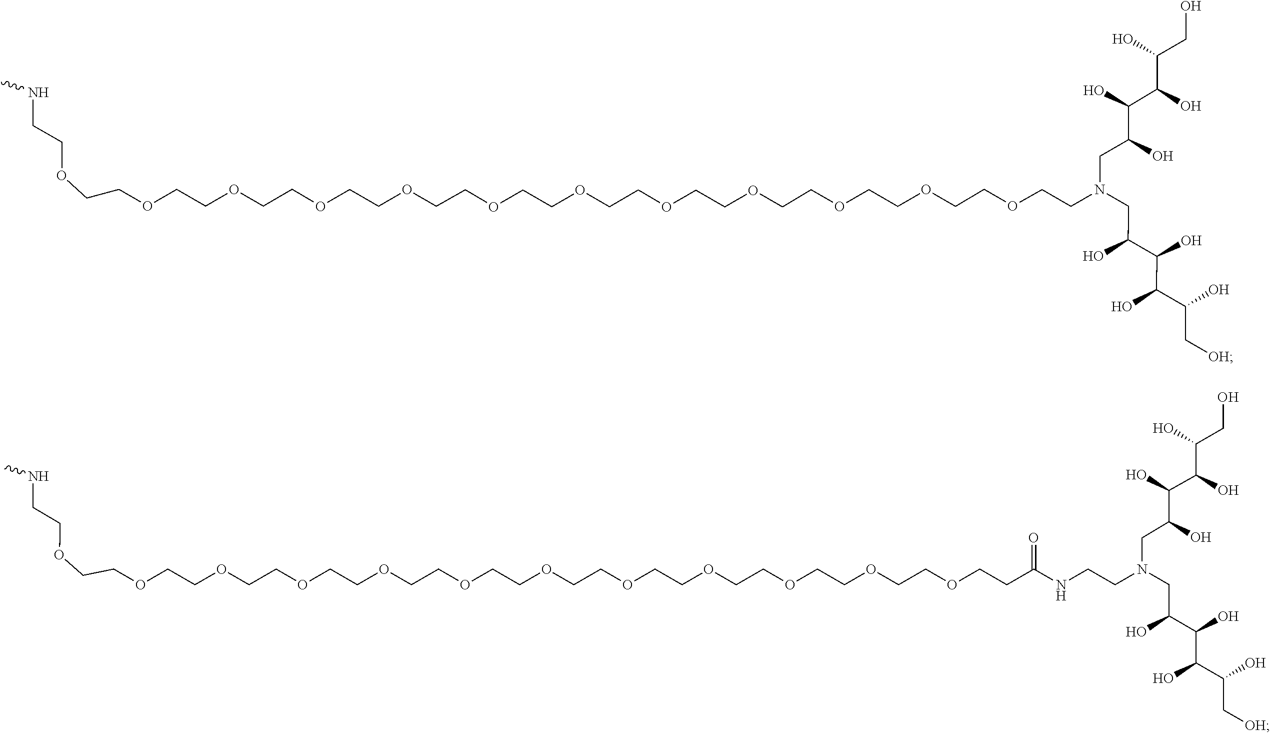

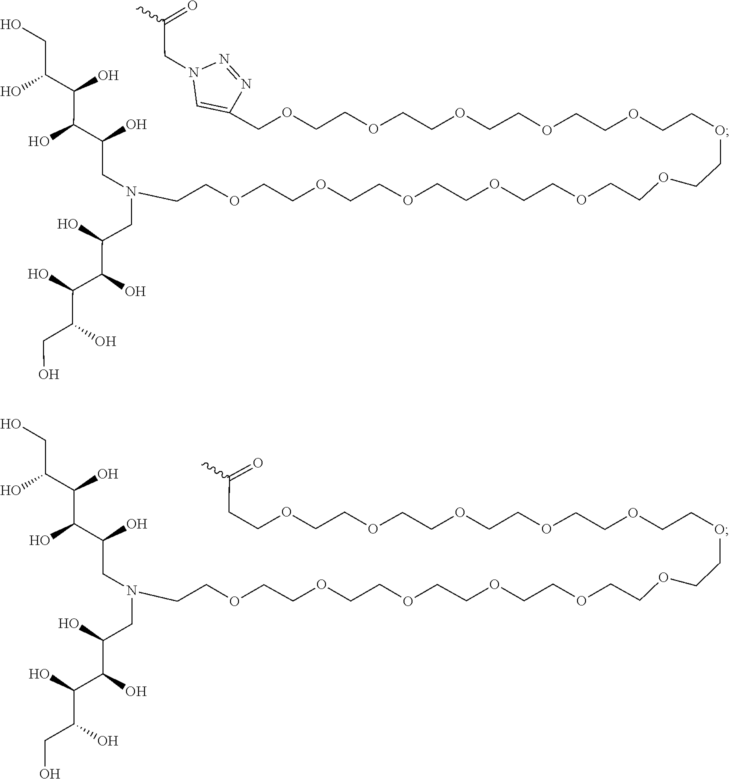

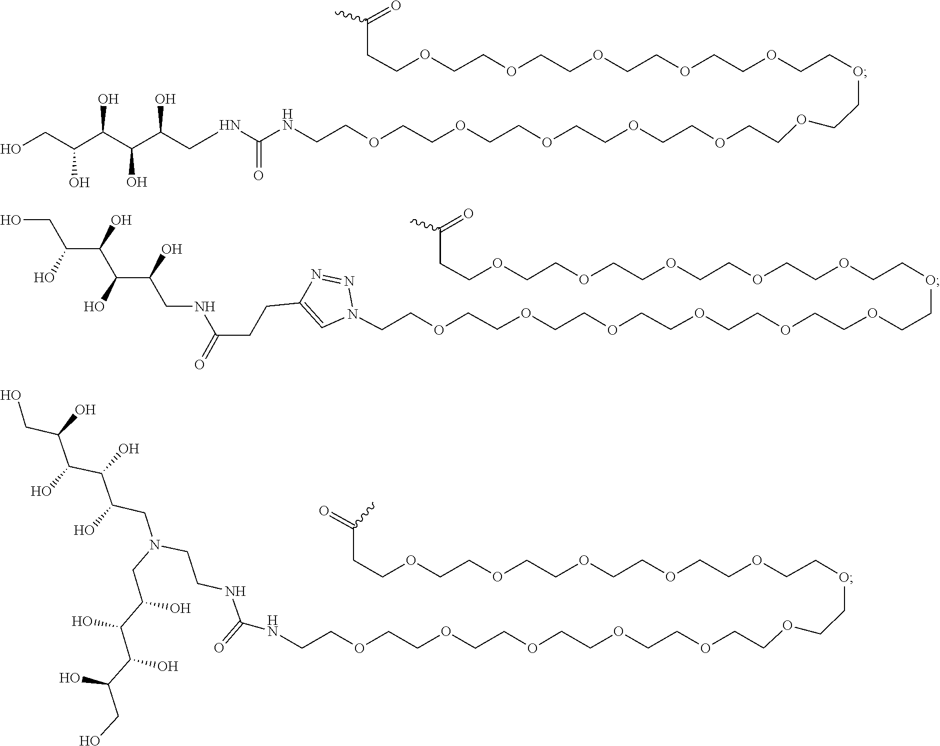

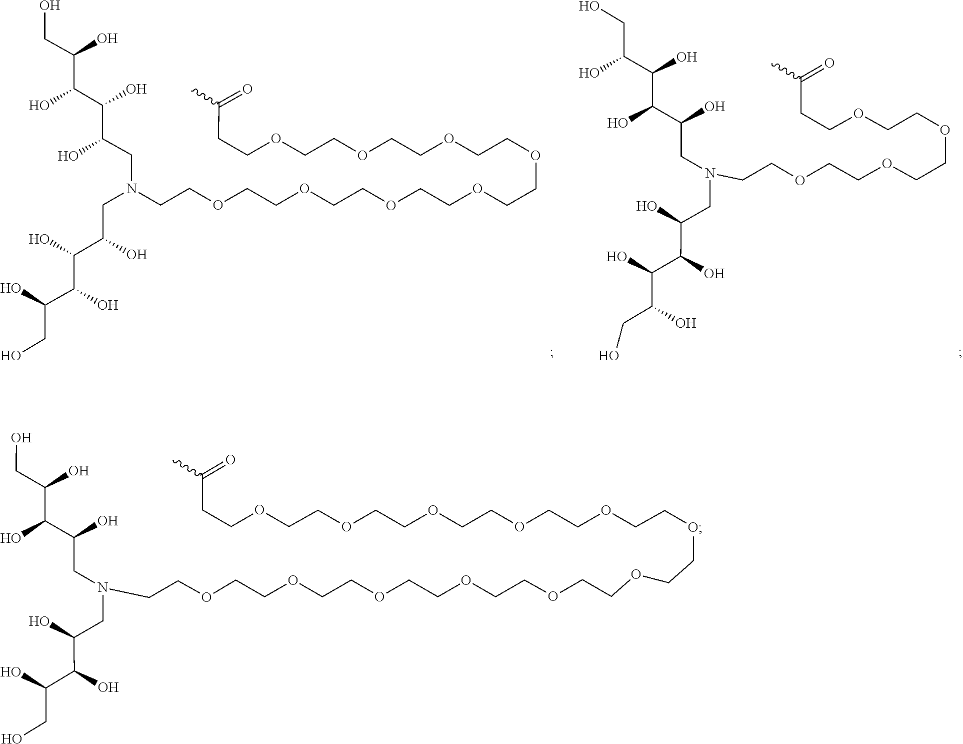

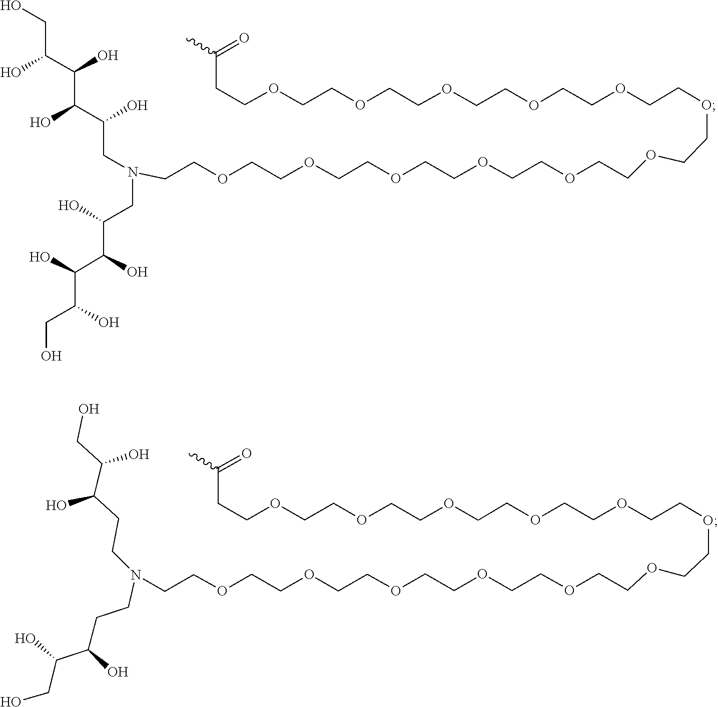

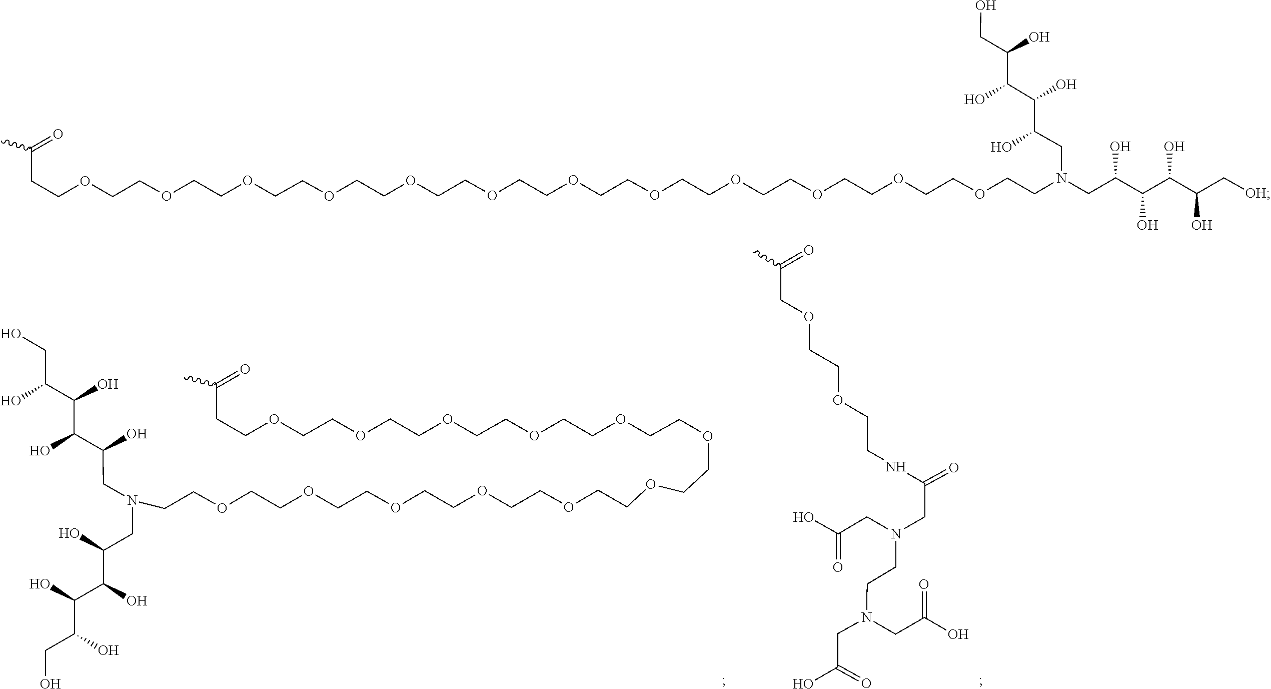

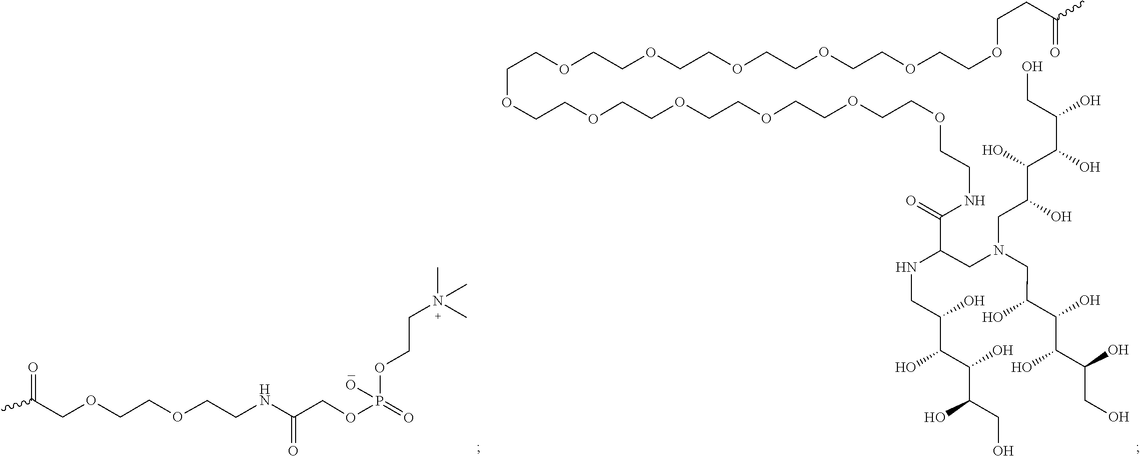

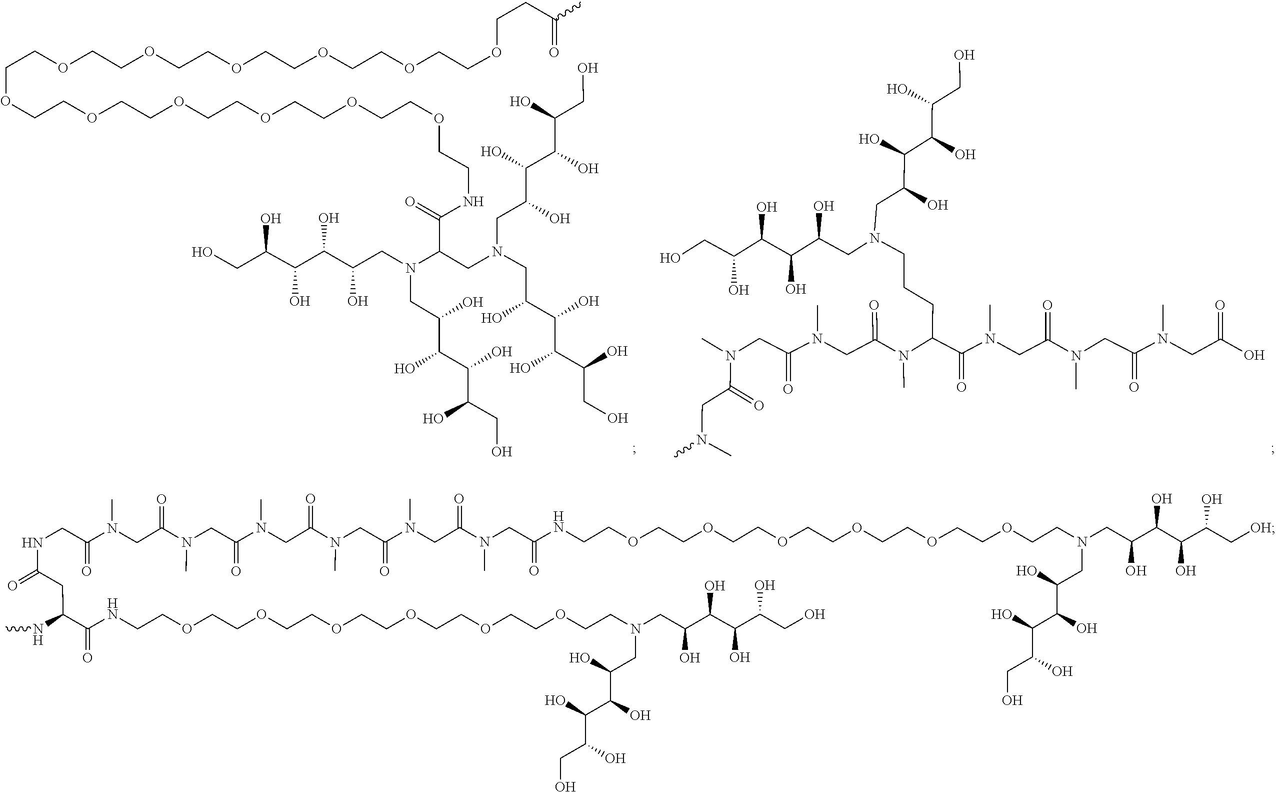

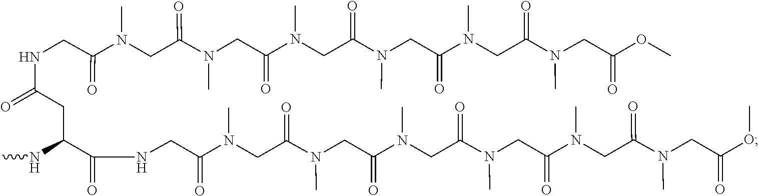

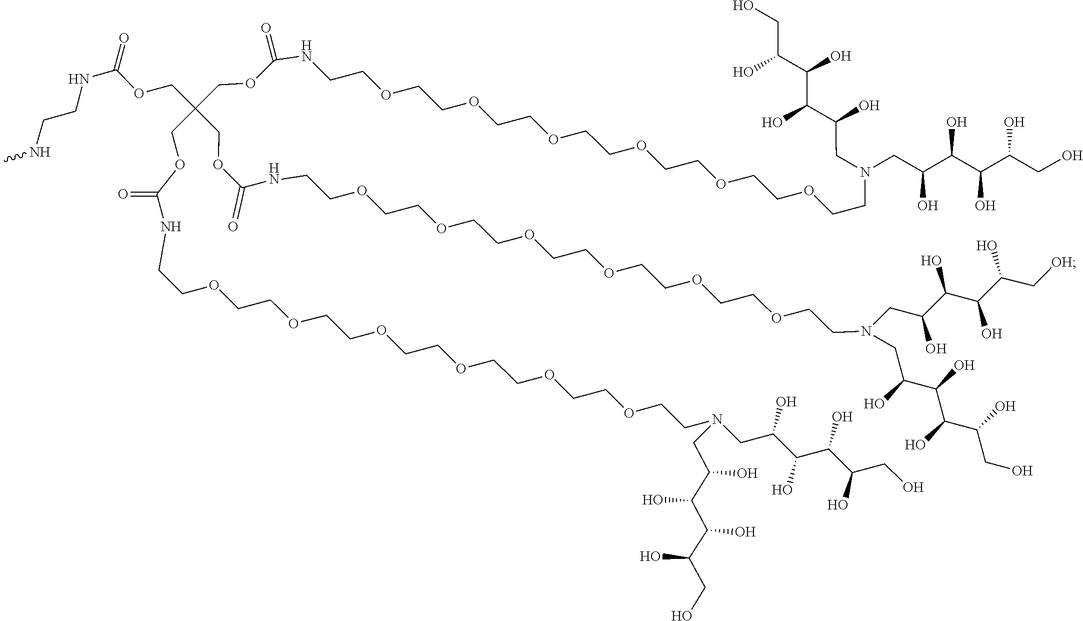

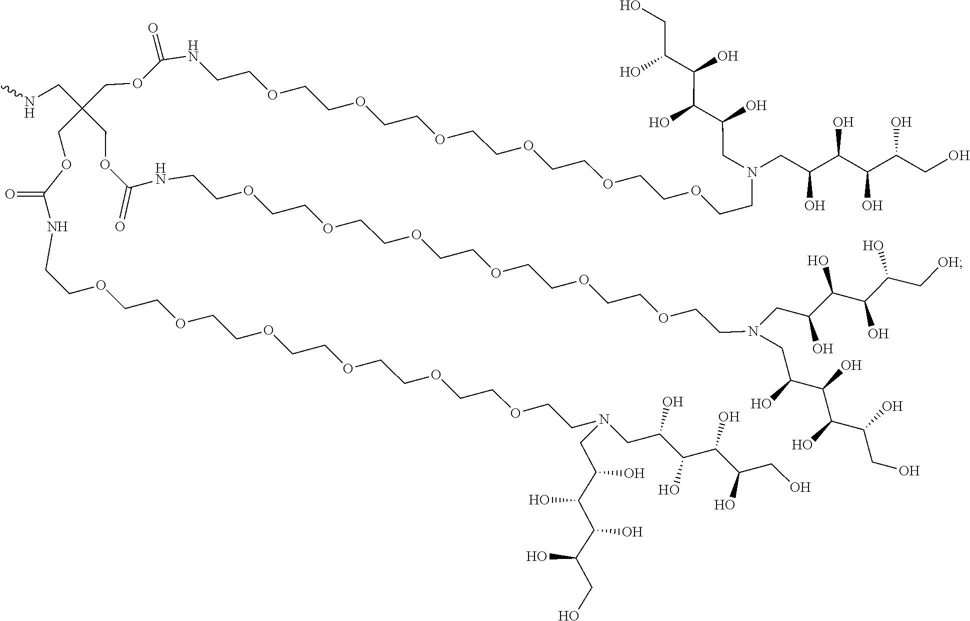

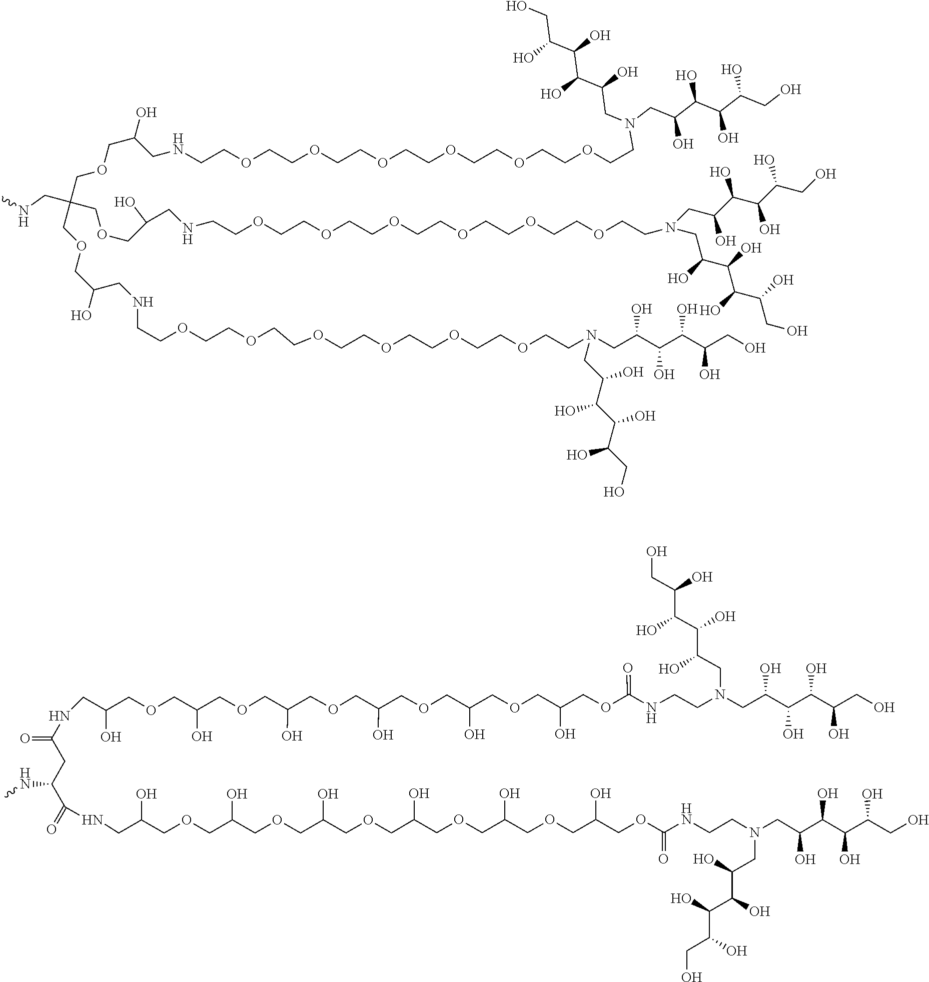

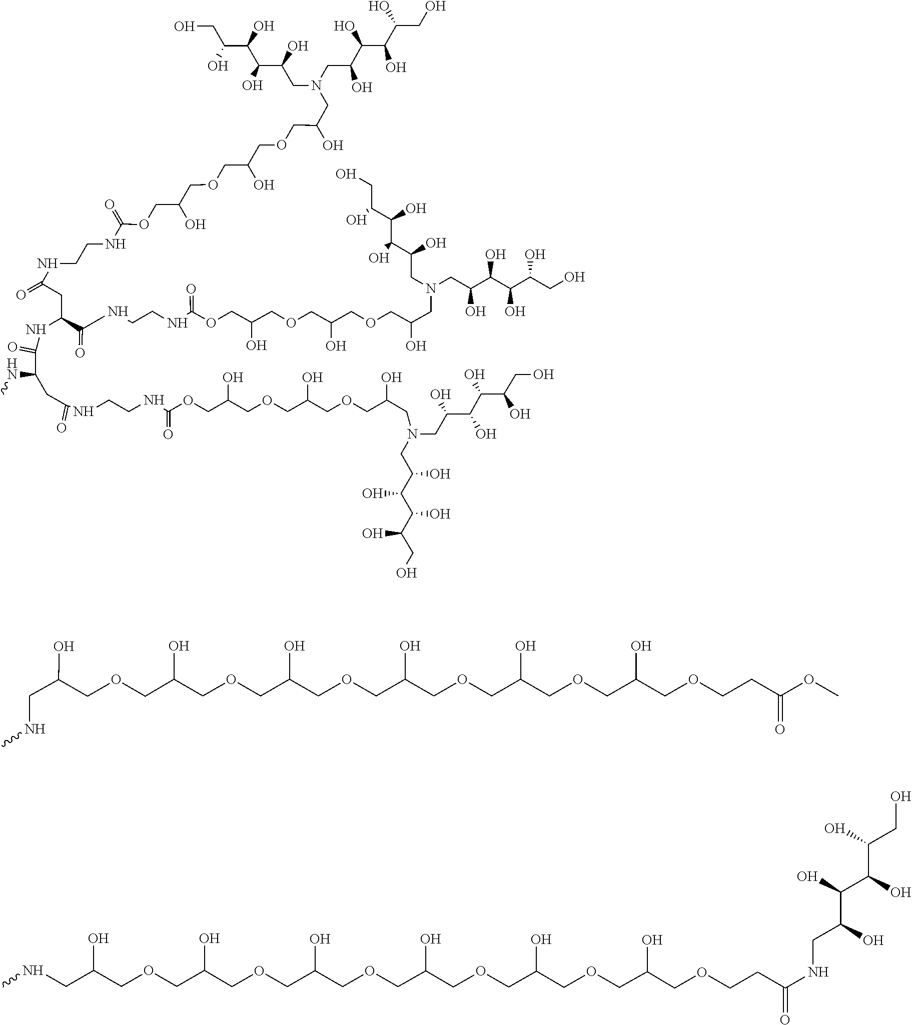

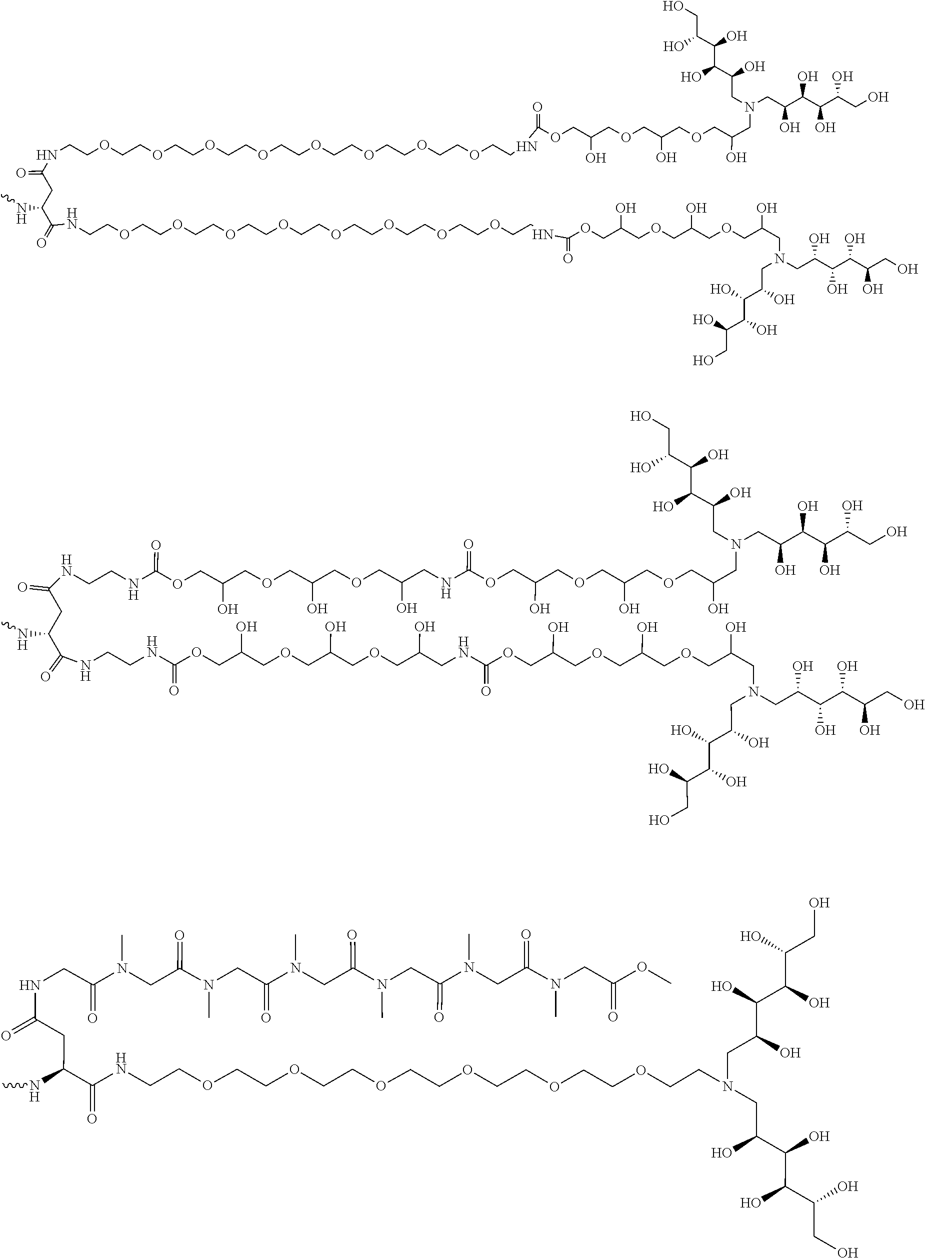

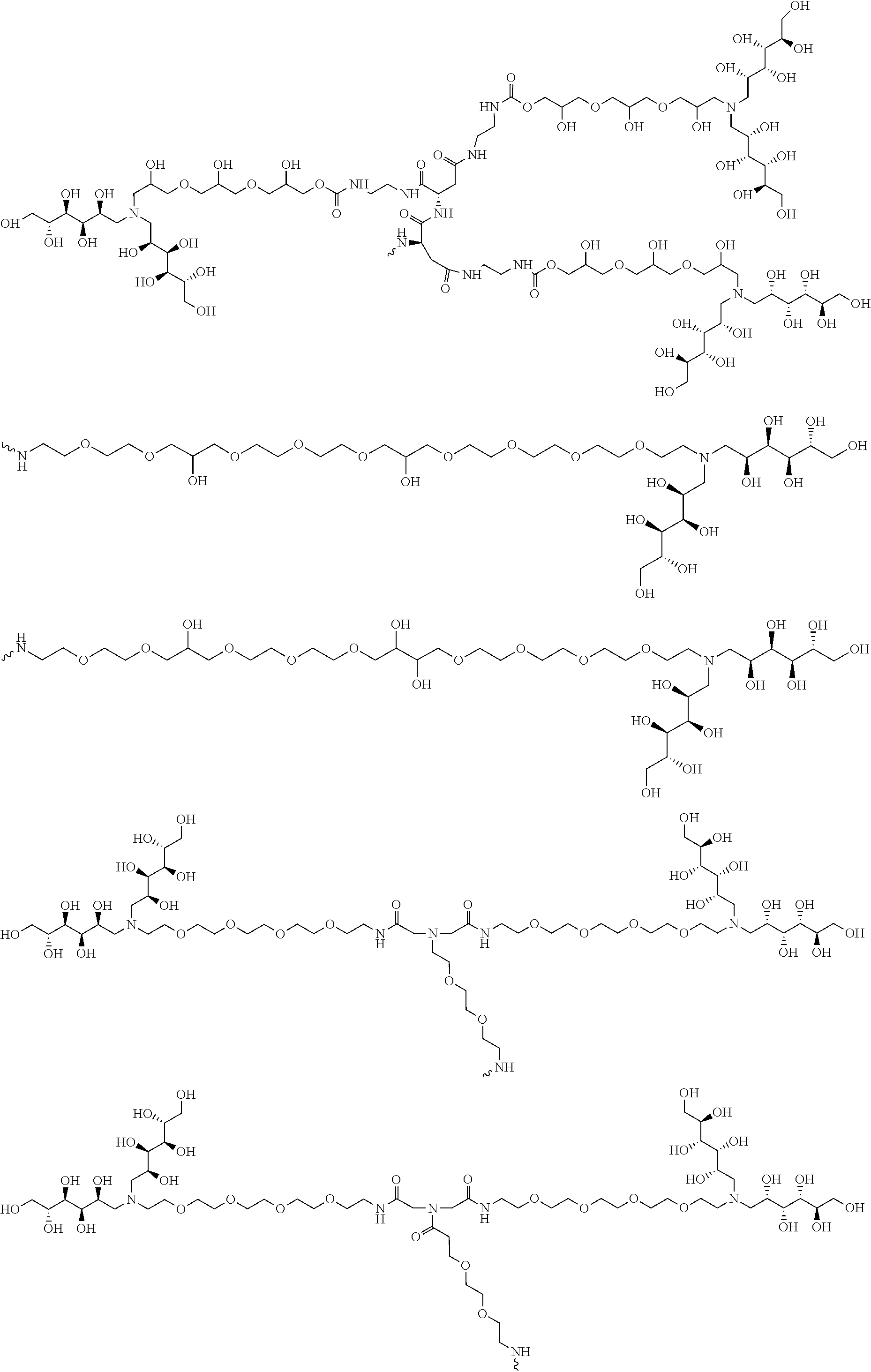

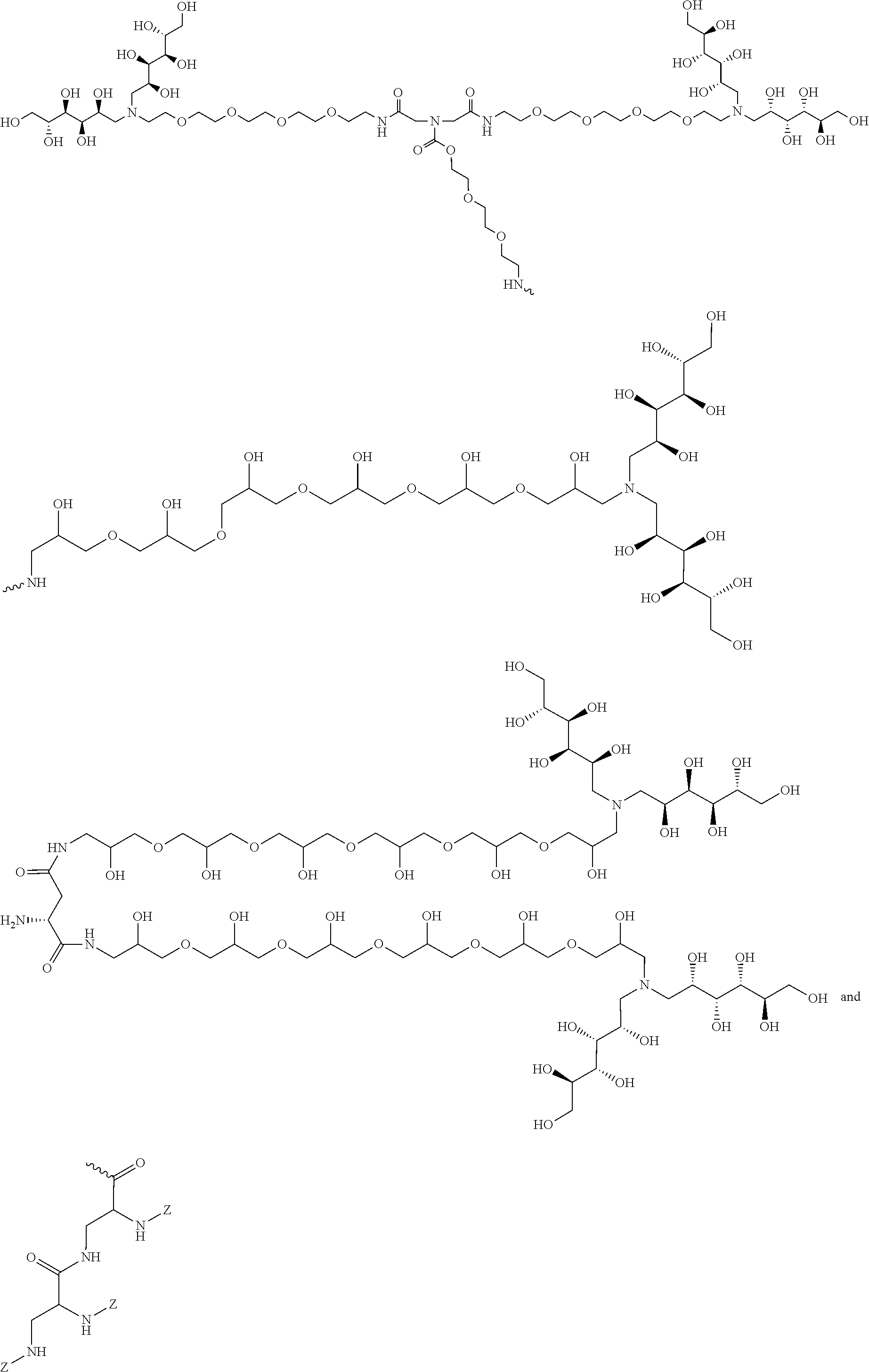

wherein each Z is attached at * and is individually selected from:

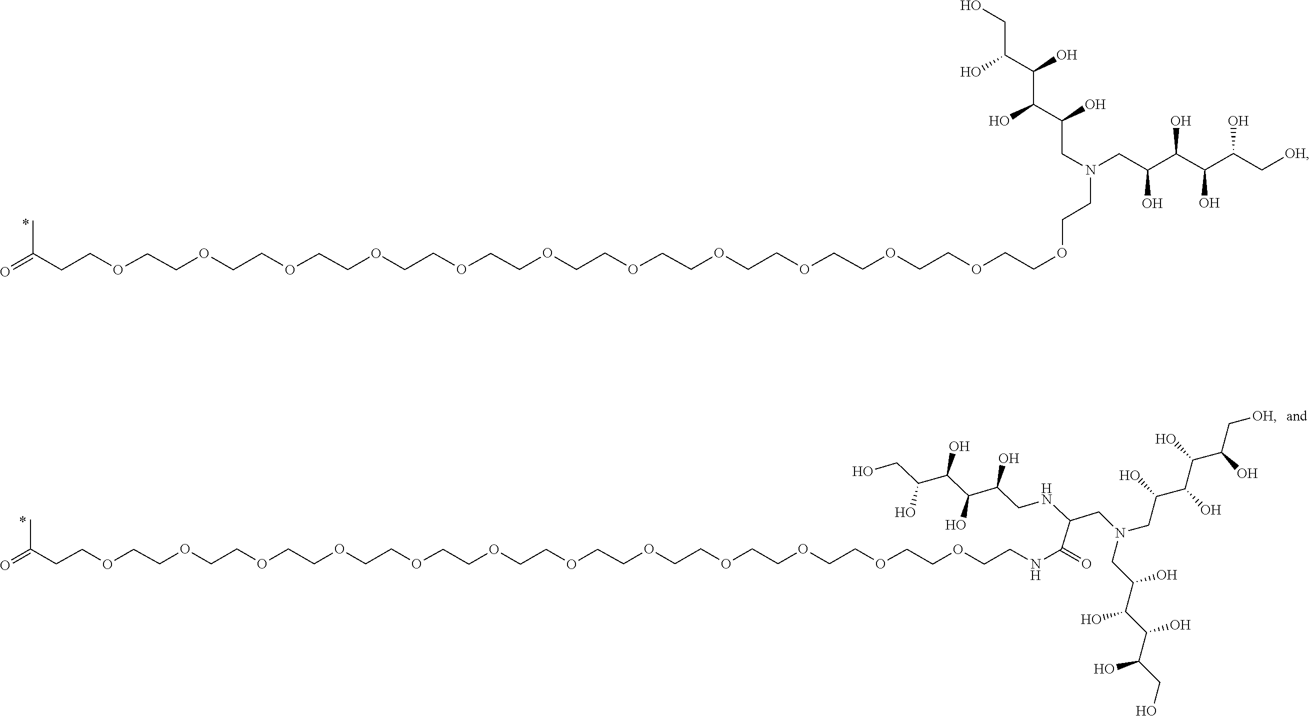

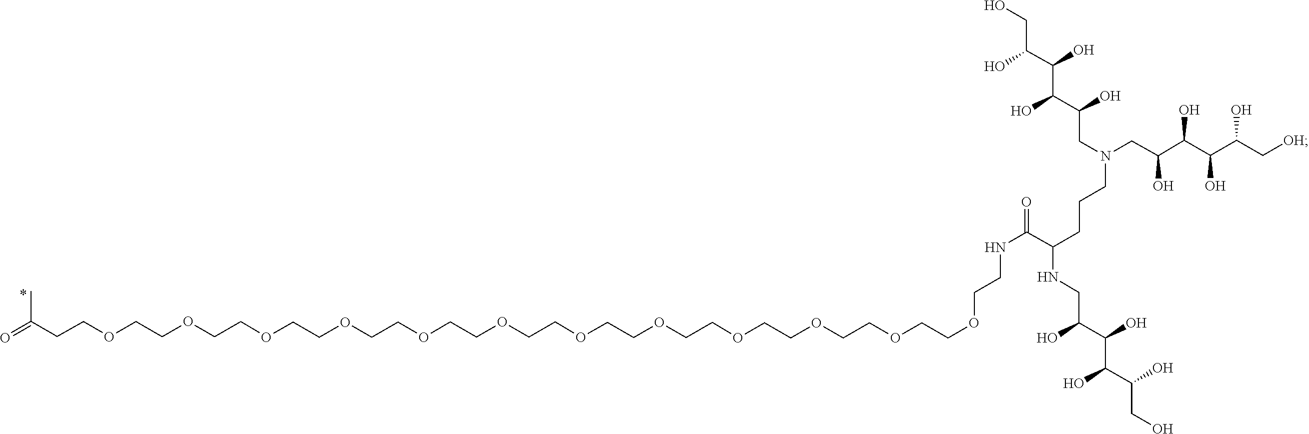

wherein each [symbol] is an attachment site to R^{b}, or to the enzyme-cleavable group.

In some embodiments, provided are Linker compounds comprising a Polar group comprising at least one Carboxyl unit having the following formula:

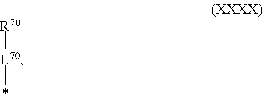

or a stereoisomer or salt thereof, wherein:
(a) L⁷⁰ is selected from C₁-C₈ alkylene, C₁-C₈ alkylene-C(O)—, —C(O)—C₁-C₈ alkylene-, and —C(O)—C₁-C₈ alkylene-C(O)—, and * is an attachment site to R^{b}, to the enzyme-cleavable group, or to a remainder of the Polar group; R⁷⁰ is ˜NR⁷¹(R⁷²-R⁷³), wherein R⁷¹ is selected from H, C₁-C₁₂ alkyl, substituted C₁-C₁₂ alkyl, or polyethylene glycol (optionally having 1 to 12 ethylene glycol subunits), R⁷² is a bond or is selected from optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl, and R⁷³ is a carboxyl or polycarboxyl, wherein polycarboxyl comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide; or
(b) L⁷⁰ is selected from C₁-C₈ alkylene, C₁-C₈ alkylene-C(O)—, —C(O)—C₁-C₈ alkylene-, and —C(O)—C₁-C₈ alkylene-C(O)—, and * is an attachment site to R^{b}, to the enzyme-cleavable group, or to a remainder of the Polar group; R⁷⁰ is —NR⁷¹(R⁷⁵-(R⁷³)₂), wherein R⁷¹ is selected from H, C₁-C₁₂ alkyl, substituted C₁-C₁₂ alkyl, or polyethylene glycol (optionally having 1 to 12 ethylene glycol subunits), R⁷⁵ is a branched optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl and each R⁷³ is independently carboxyl or polycarboxyl, wherein the polycarboxyl comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amidelr
(c) L⁷⁰ is selected from C₁-C₈ alkylene, C₁-C₈ alkylene-C(O)—, —C(O)—C₁-C₈ alkylene-, and —C(O)—C₁-C₈ alkylene-C(O)—, and * is an attachment site to R^{b}, to the enzyme-cleavable group, or to a remainder of the Polar group; R⁷⁰ is —N(R⁷⁴-R⁷³)(R⁷²-R⁷³), wherein R⁷² and R⁷⁴ are each independently selected from optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl, and each R⁷³ is independently carboxyl or polycarboxyl, wherein the polycarboxyl comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide.

In some embodiments, provided are Linker compounds comprising a Polar group including the Polymer unit and a Sugar unit. In other embodiments, provided are Linker compounds comprising a Polar group including at least two Polymer units. In other embodiments, provided are Linker compounds comprising a Polar group including the Polymer unit and a Carboxyl unit. In other embodiments, provided are Linker compounds comprising at least two Polar groups. In other embodiments, provided are Linker compounds comprising a Polar group including the Polymer unit, the Sugar unit and the Carboxyl unit. In still other embodiments, provided are Linker compounds comprising a Polar group including at least two Polymer units, at least one Sugar unit and at least one Carboxyl unit.

In some embodiments, provided are Linker compounds comprising at least one of the Polar group attached to the enzyme-cleavable group. For example, in some embodiments provided are Linker compounds comprising at least one Polar group and an the enzyme-cleavable group comprising at least two amino acids.

### Linker Unit

In some embodiments, the Linker unit has one of the following structures (i) or (ii):

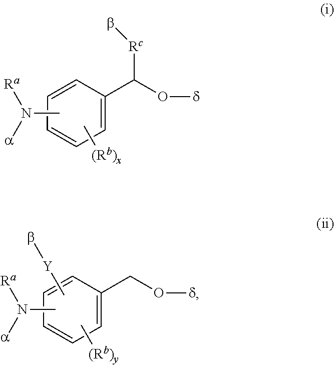

wherein:
α— is an attachment site to an enzyme-cleavable group;
β— is an attachment site to the at least one Polar group;
δ— is H, an attachment site to at least one of the Drug units, or an attachment site to a linking group attached to the at least one of the Drug units;
each R^{α} independently is H or C₁-C₆ alkyl;
each R^{b} independently is halo, C₁₋₆ alkyl, an attachment site to at least one of the Drug units, or an attachment site to at least one of the Polar group;
x is 0, 1, 2, 3 or 4;
y is 0, 1, 2 or 3;
R^{c} is a bond, —C(O)—, —S(O)—, —SO₂—, C₁₋₆ alkylene, C₁₋₆ alkynylene, triazolyl, or combinations thereof; and
Y is a bond, —O—, —S—, —N(R^{a})—, —C(O)—, —S(O)—, —SO₂—C₁-C₆ alkylene, C₁-C₆ alkenylene, C₁-C₆ alkynylene, a group containing triazolyl, or combinations thereof.

In some embodiments, the Linker unit has from 1 to 4 attachment sites for a Drug unit. In some embodiments, the Linker unit has from 1 to 3 or 1 to 2 attachment sites for a Drug unit (D).

In some embodiments, a Linker unit includes at least one Polar group, such as a Polymer unit. In some embodiments, the Polar group includes at least one Polymer unit and optionally a Sugar unit and/or Carboxyl unit or combinations thereof. In some embodiments, the Polymer unit comprises a polyamide, a polyether, or a combination thereof, wherein the polyether comprises a hydroxyl group, a polyhydroxyl group, a sugar group, a carboxyl group, or combinations thereof.

In some embodiments, the Linker unit is a cleavable linker subunit. As used herein, the term “cleavable” refers to a metabolic process or reaction inside a cell or in the extracellular milieu, whereby the covalent attachment between a Drug unit (e.g., a cytotoxic agent) and the Linker unit or portion thereof is broken, resulting in the free Drug unit, or a metabolite of the Linker unit-Drug, which is dissociated from the remainder of the Linker unit.

In some embodiments, the Linker unit includes a protease cleavable linker subunit. In some embodiments, the Linker unit is a protease cleavable linker that is cleavable under intracellular conditions, such that cleavage of or within the Linker unit releases the Drug unit from Linker unit or the remainder of Linker unit in the intracellular environment. For example, in some embodiments, the Linker unit is cleavable by a cleaving agent that is present in the intracellular environment (e.g., within a lysosome or endosome or caveolae). As used herein, the terms “cleavable under intracellular conditions,” “intracellularly cleaved,” and “intracellular cleavage” refer to a metabolic process or reaction inside a cell, whereby the covalent attachment between a Drug unit (e.g., a cytotoxic agent) and the Linker unit or portion thereof is broken, resulting in the free Drug unit, or ar metabolite of the Linker unit-Drug unit dissociated from the remainder of the Linker unit inside the cell. One advantage of using intracellular proteolytic release of the Drug unit is that the activity of the Drug unit is typically attenuated when conjugated and the serum stabilities of the conjugates are typically high.

In some embodiments, a linkage between the Linker unit and the Drug unit can be cleaved by enzymatically cleavage within the Linker unit by one or more enzymes, including a tumor-associated protease, to liberate the Drug unit. The Linker unit can be, for example, a peptidyl linker that is cleaved by an intracellular peptidase or protease enzyme, including, but not limited to, a lysosomal or endosomal protease. Intracellular protease or cleaving agents can include cathepsins B, C and D and plasmin, all of which are known to hydrolyze dipeptide drug derivatives resulting in the release of active drug inside target cells (see, e.g., Dubowchik and Walker, 1999, Pharm. Therapeutics 83:67-123). Peptidyl linkers can be cleavable by enzymes that are present in target antigen-expressing cells. For example, a Linker unit that is cleavable by the thiol-dependent protease cathepsin-B, which is highly expressed in cancerous tissue, can be used (e.g., having a Phe-Leu, Val-Ala, Val-Cit or Gly-Phe-Leu-Gly peptide).

Typically, a Linker unit has at least one amino acid or at least two amino acids that form a recognition site for a protease or other cleaving agent. In certain embodiments, the Linker unit has a dipeptide, tripeptide, tetrapeptide or pentapeptide. In certain embodiments, a Linker unit can comprise only natural amino acids. In some embodiments, For example, a Linker unit can have a Phe-Leu, Val-Ala, or Val-Cit peptide that is attached to a PABA group of structure (i) or (ii). Other such cleavable Linker units are described, for example, in U.S. Pat. No. 6,214,345, WO2004/010957, US20150297748, US2008/0166363, US20120328564 and US20200347075, each of which is incorporated by reference herein. In some embodiments, a Linker unit that is cleavable by an intracellular protease comprises a Val-Cit peptide or a Phe-Lys peptide or Gly-Gly-Phe-Gly linker (see, e.g., US Published Application No. 2015/0297748).

In some embodiments, a Linker unit contains one or more the following: glycine and/or L-amino acids, such as arginine, glutamine, phenylalanine, tyrosine, tryptophan, lysine, alanine, histidine, serine, proline, glutamic acid, aspartic acid, threonine, cysteine, methionine, leucine, asparagine, isoleucine, and valine, that form a recognition and cleavage site for a protease or other cleaving enzyme.

In some embodiments, an amino acid of a Linker unit has the formula denoted below in the square brackets:

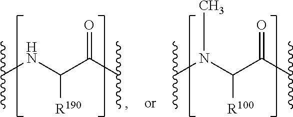

wherein R₁₉₀ is hydrogen, methyl, isopropyl, isobutyl, sec-butyl, benzyl, p-hydroxybenzyl, —CH₂OH, —CH(OH)CH₃, —CH₂CH₂SCH₃, —CH₂CONH₂, —CH₂COOH —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-, phenyl, cyclohexyl,

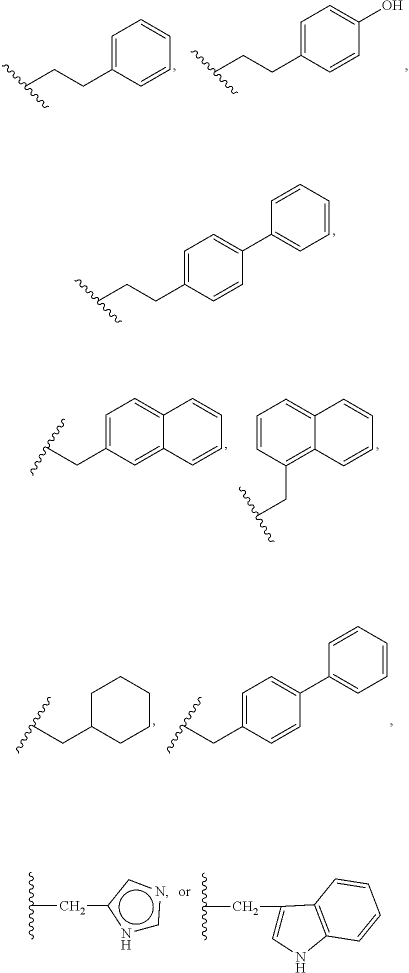

In some embodiments, a Linker unit includes one or more of the following L-(natural) amino acids: alanine, arginine, aspartic acid, asparagine, histidine, glycine, glutamic acid, glutamine, phenylalanine, lysine, leucine, serine, tyrosine, threonine, isoleucine, tryptophan and valine. In some embodiments, a Linker unit does not contain cysteine. In some embodiments, a peptidyl linker does not contain proline.

In some embodiments, a Linker unit includes one or more of the following amino acids: alanine, arginine, aspartic acid, asparagine, histidine, glycine, glutamic acid, glutamine, phenylalanine, lysine, leucine, serine, tyrosine, threonine, isoleucine, proline, tryptophan, valine, ornithine, penicillamine, β-alanine, aminoalkanoic acid, aminoalkynoic acid, amino alkanedioic acid, aminobenzoic acid, amino-heterocyclo-alkanoic acid, heterocyclo-carboxylic acid, citrulline, statine, diaminoalkanoic acid, and derivatives thereof.

In some embodiments, a Linker unit includes protease cleavable linker comprising a thiol-reactive maleimidocaproyl spacer or Stretcher, an amino acid or peptide and a self-immolative group. In some embodiments, a Linker unit includes protease cleavable linker comprising a thiol-reactive maleimidocaproyl spacer, a valine-citrulline dipeptide, and a p-amino-benzyloxycarbonyl self immolative group.

In some embodiments, a Linker unit includes a self-stabilizing moiety comprising a maleimide group as described in WO2013/173337.

In some embodiments, the Linker unit includes a glucuronide-cleavable moiety, see, e.g., US 2014/0031535.

In some embodiments, provided are Linker compounds having one of the following structures:

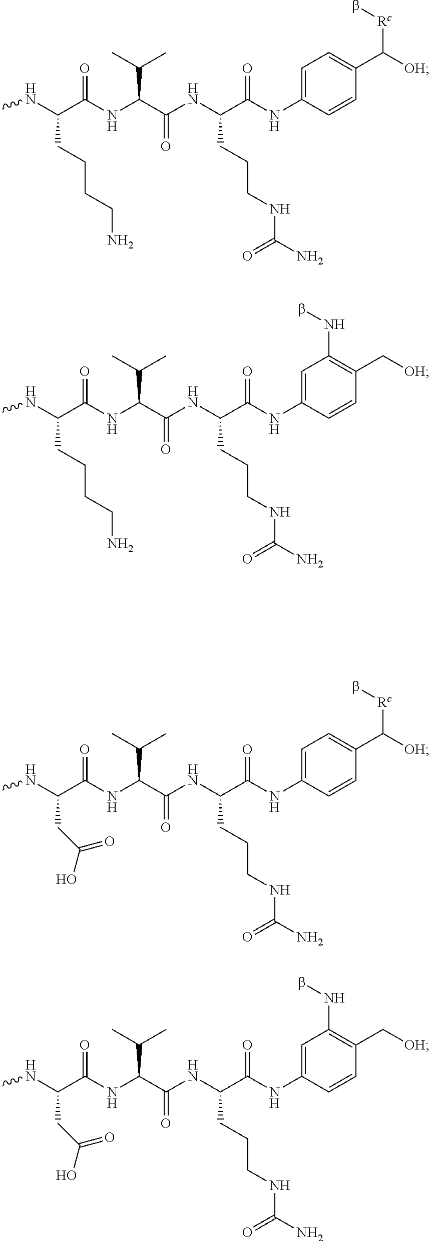

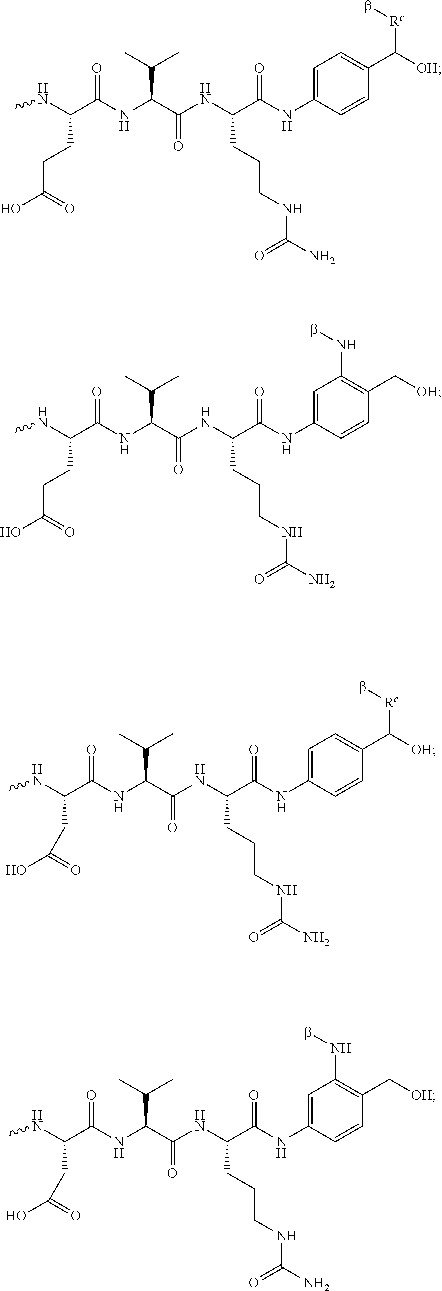

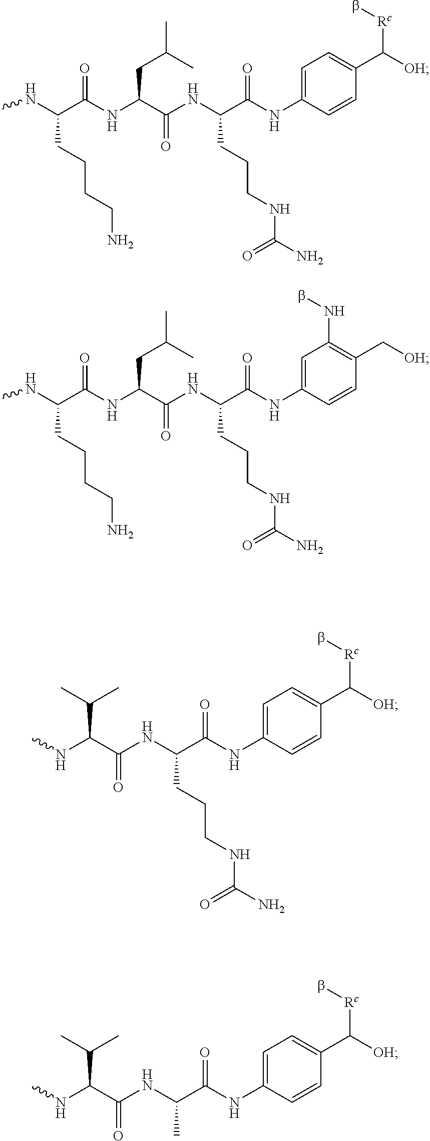

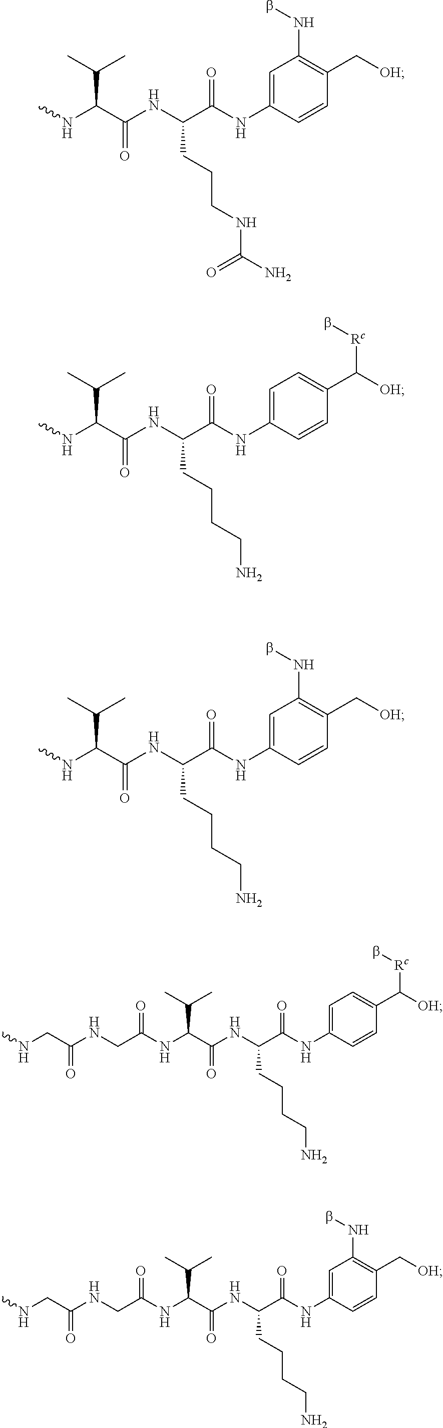

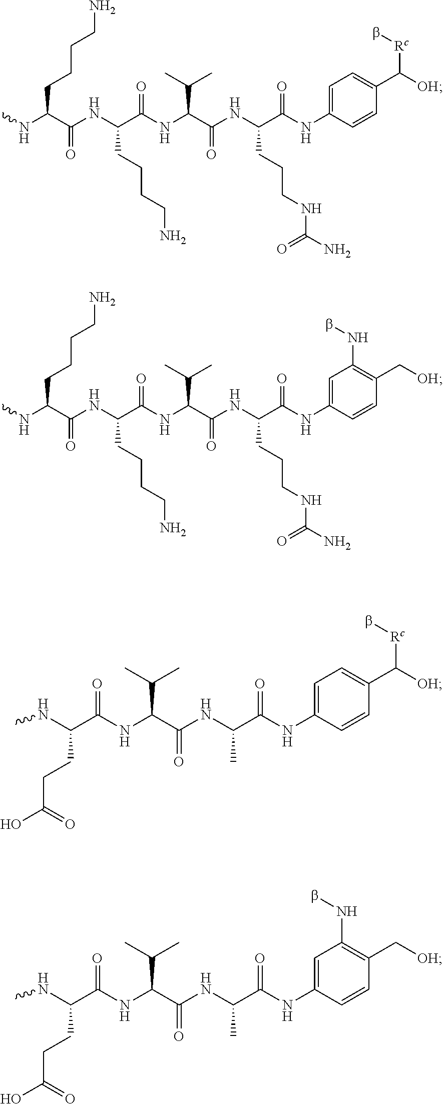

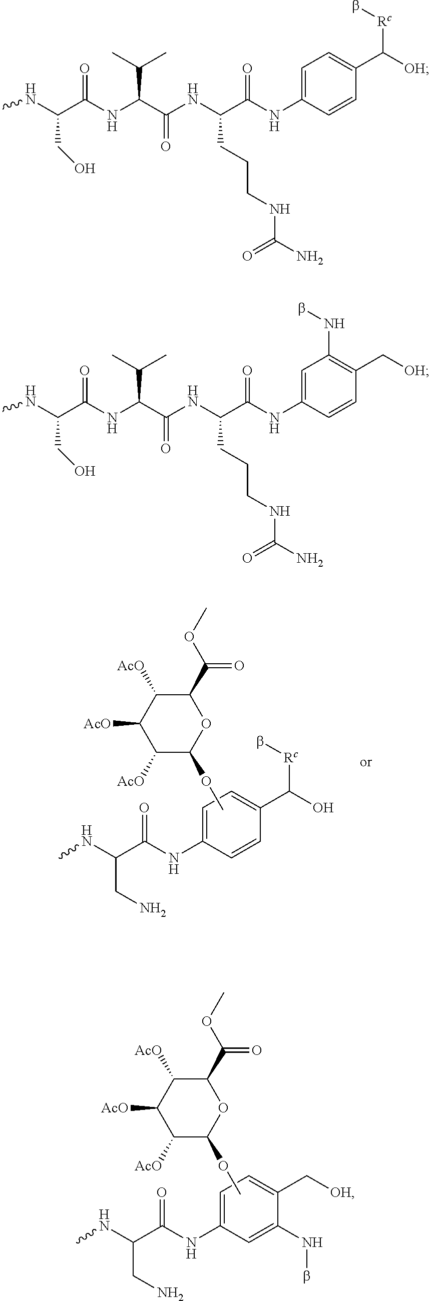

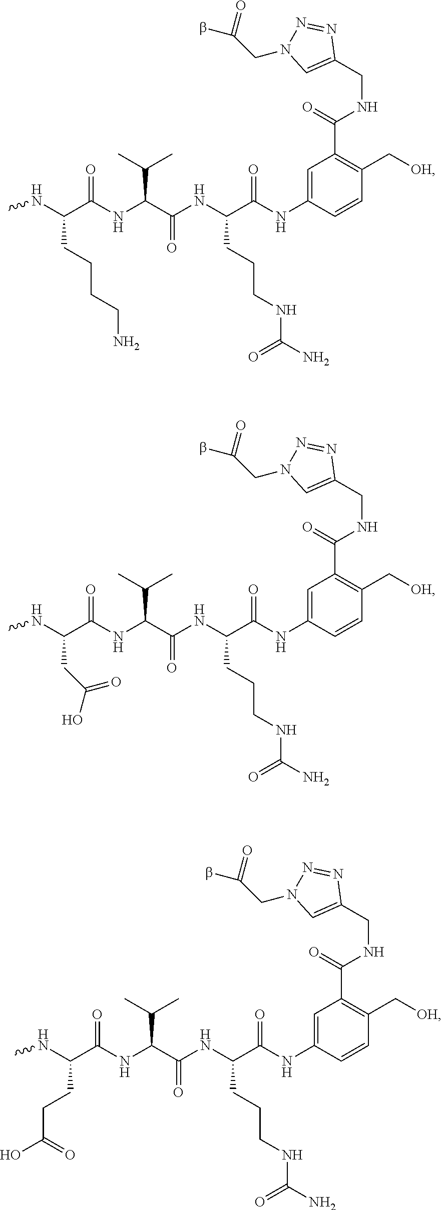

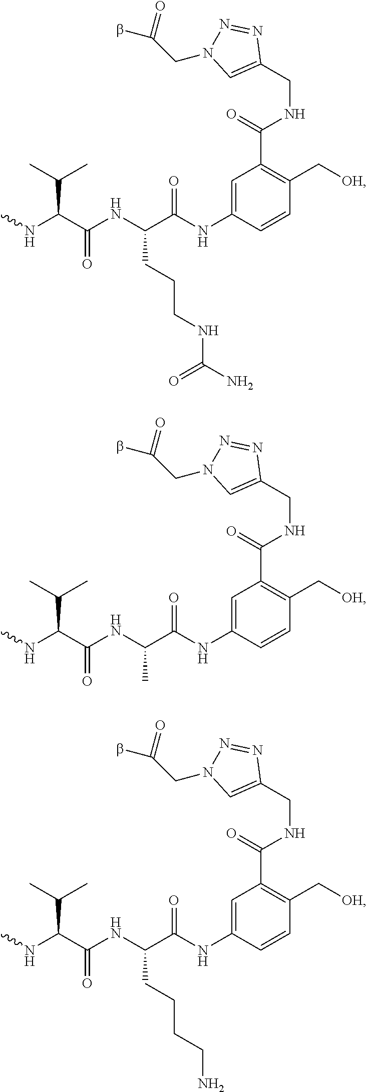

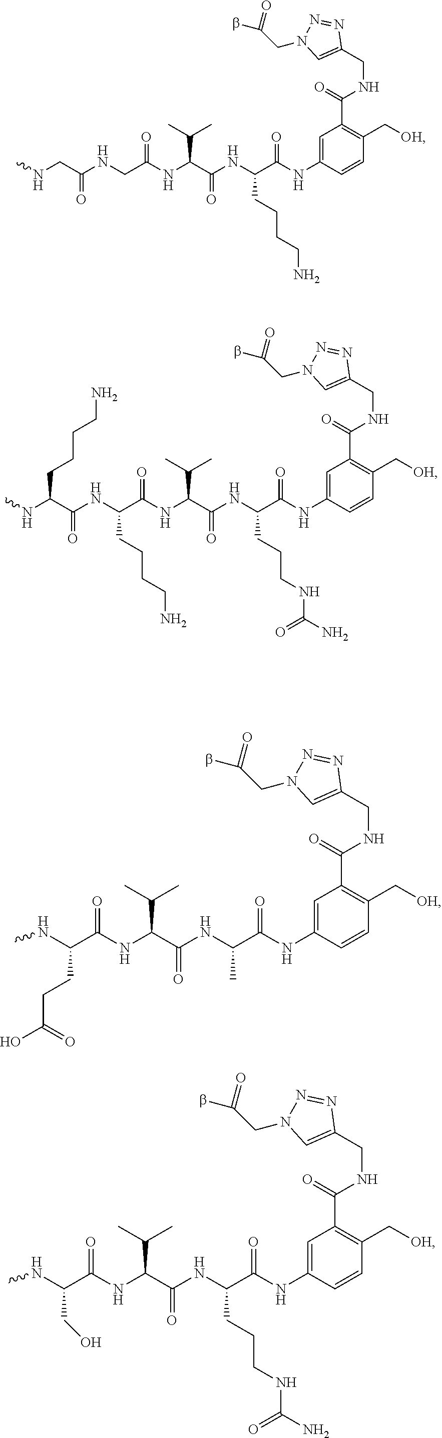

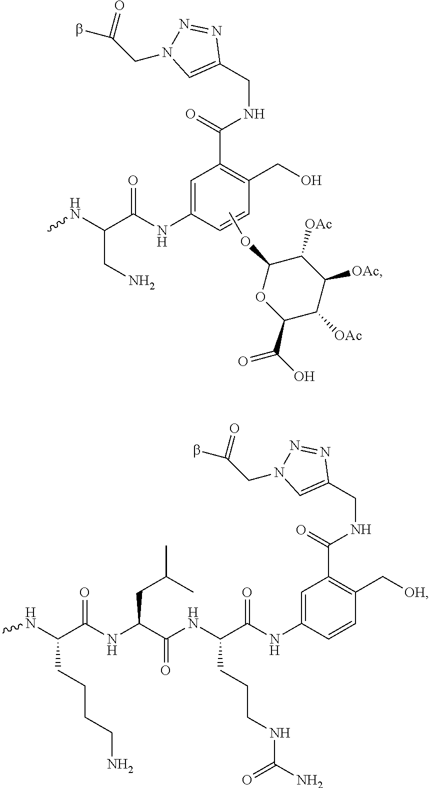

wherein: R^{c} is a bond or C₁₋₆ alkylene; the wavy line on the amino group indicates an attachment site for a Stretcher group; or, prior to attachment to the Stretcher group, indicates H; β— is the attachment site to the at least one Polar group; and the benzylic H on the benzylic OH is optionally replaced with a bond to at least one of the Drug units or to the linking group attached to at least one of the Drug units.

In some embodiments, at least one of the Drug units is attached directly to the benzylic O at δ. In some embodiments, at least one of the Drug units is attached indirectly, via a linking group. A linking group can be any suitable group for connection of the at least one Drug unit to the benzylic —O— that allows for release of an active Drug unit, or release of an active derivative of the linking group-Drug unit. In some embodiments, a linking group is —NH—CH₂—CH₂—CH₂—C(O)—, the Drug unit is exatecan and the released Drug unit is DXd. (See., e.g., Published US Application No. 2019/000898). In other embodiments, the linking group is —C(O)—NH—CH₂—CH₂—CH₂—C(O)—.

In some embodiments, provided are Linker compounds wherein the Linker unit is a cleavable linker unit. For example, in some embodiments the enzyme-cleavable group comprises a peptide that is cleavable by an intracellular protease. In some embodiments, the intracellular protease is Cathepsin B. In more specific embodiments, the cleavable peptide comprises a valine-citrulline peptide, a valine-alanine peptide, a valine-lysine peptide, a phenylalanine-lysine peptide, or a glycine-glycine-phenylalanine-glycine peptide.

In some embodiments, provided are Linker compounds comprising one of the following structures:

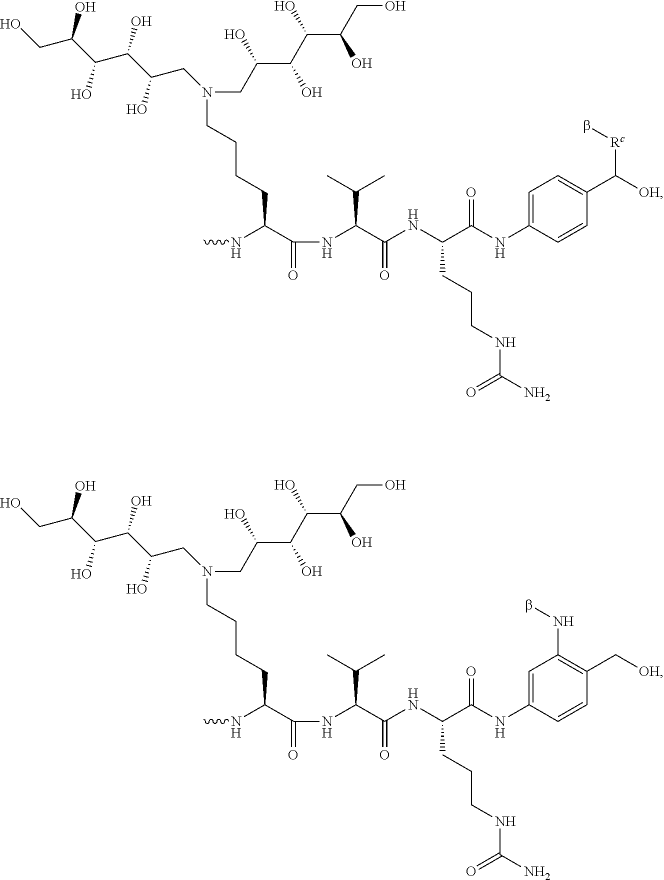

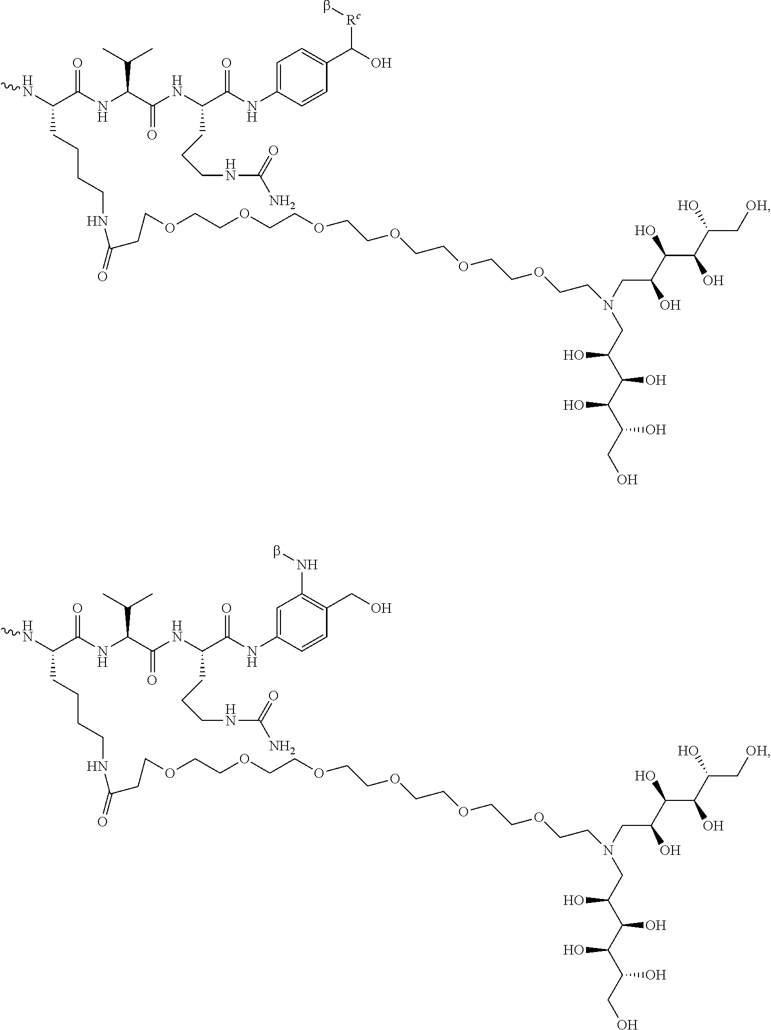

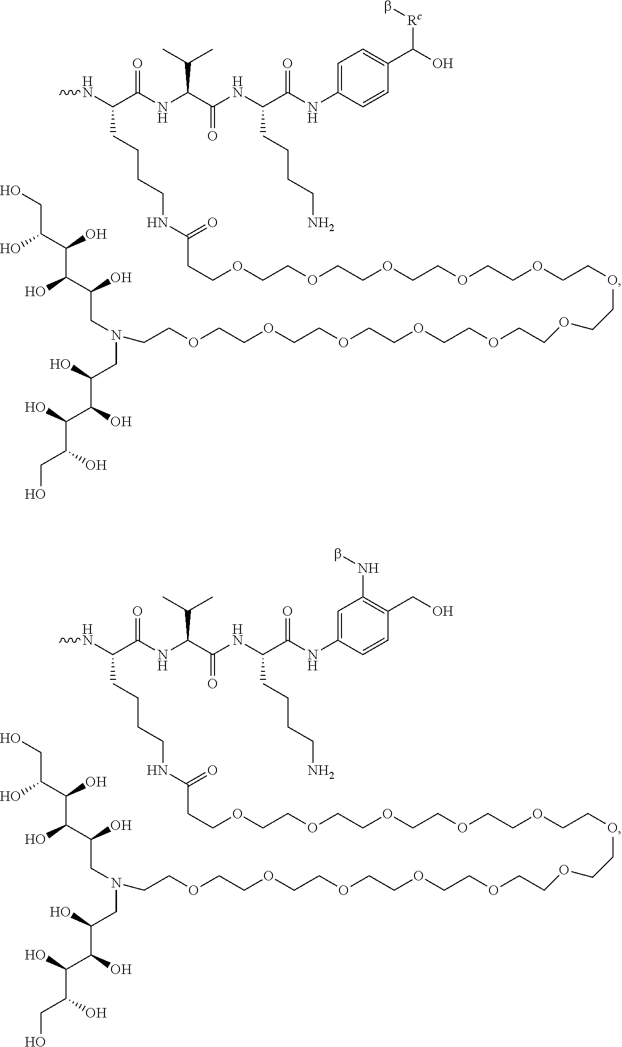

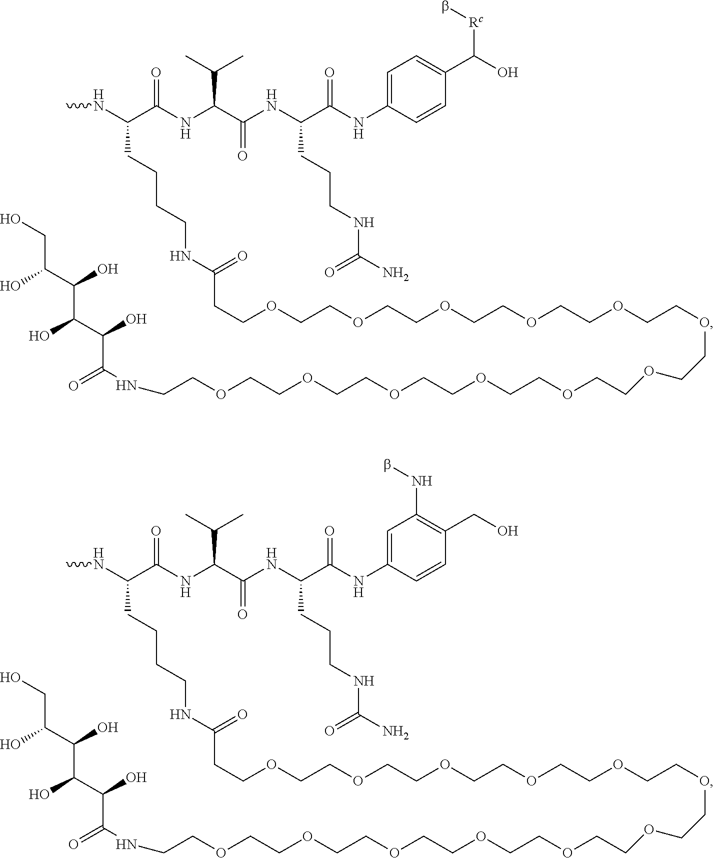

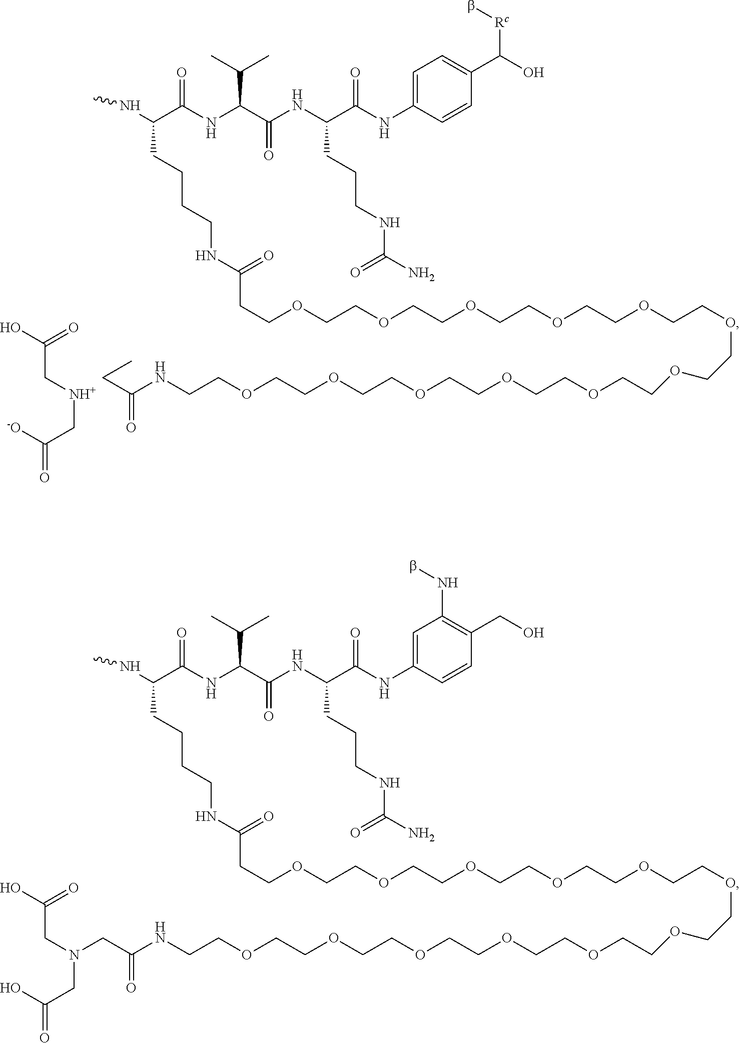

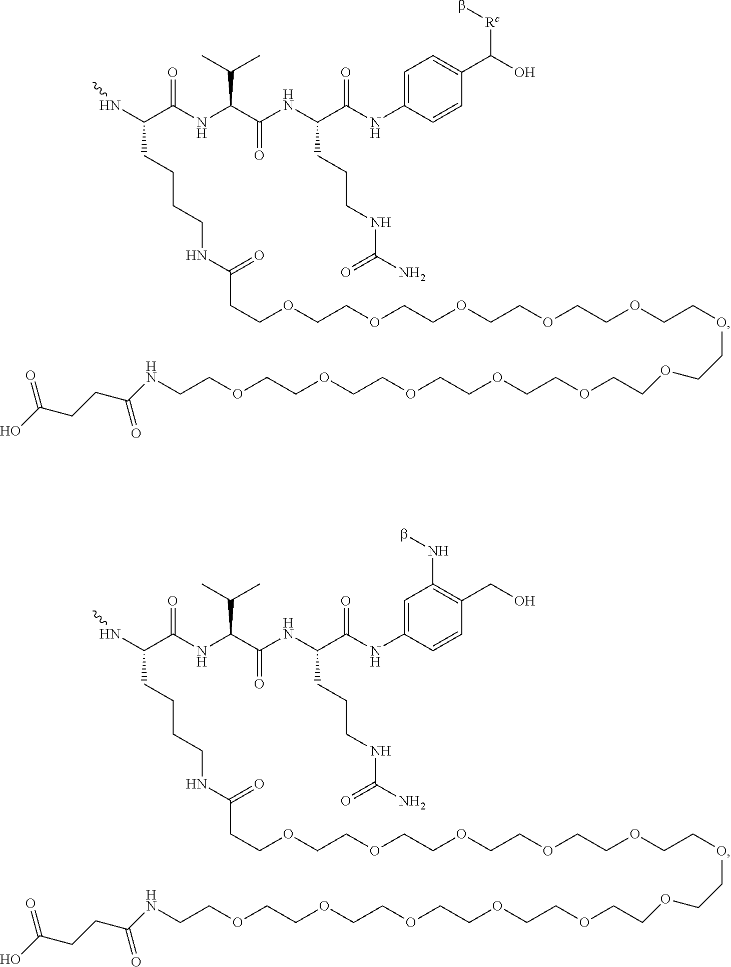

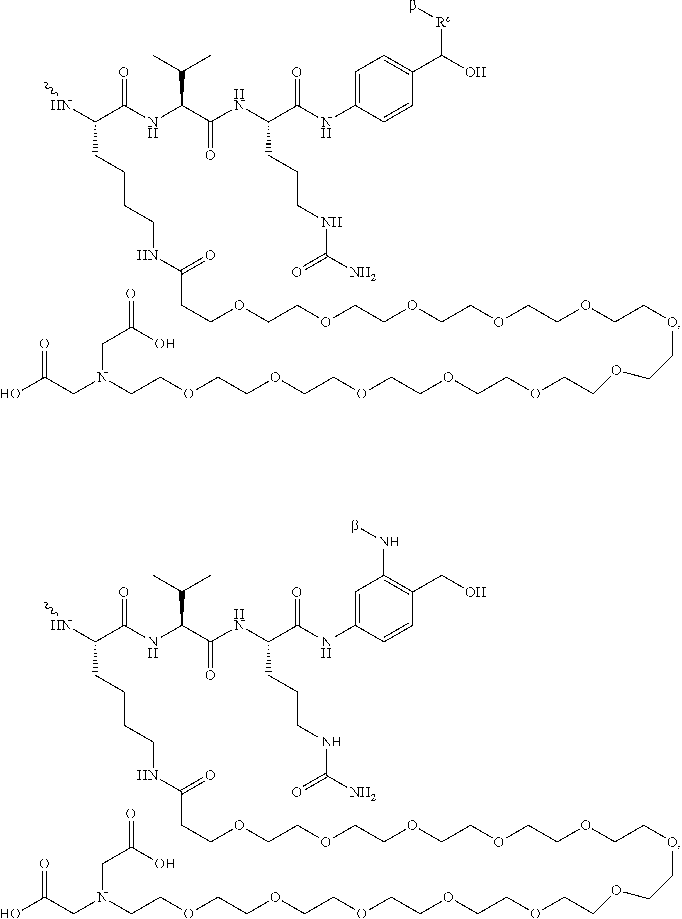

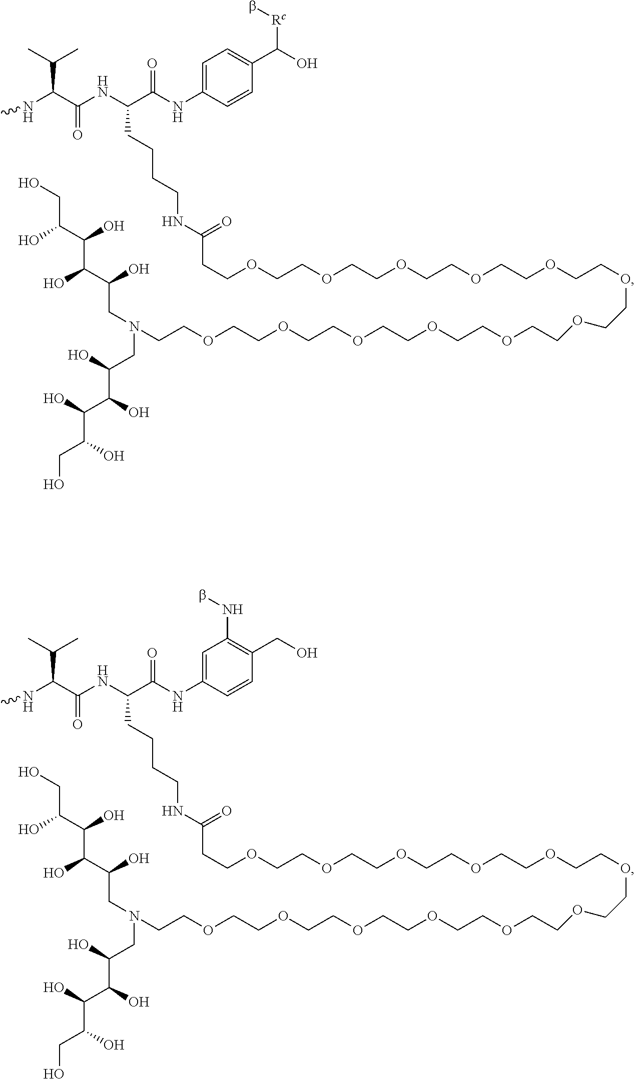

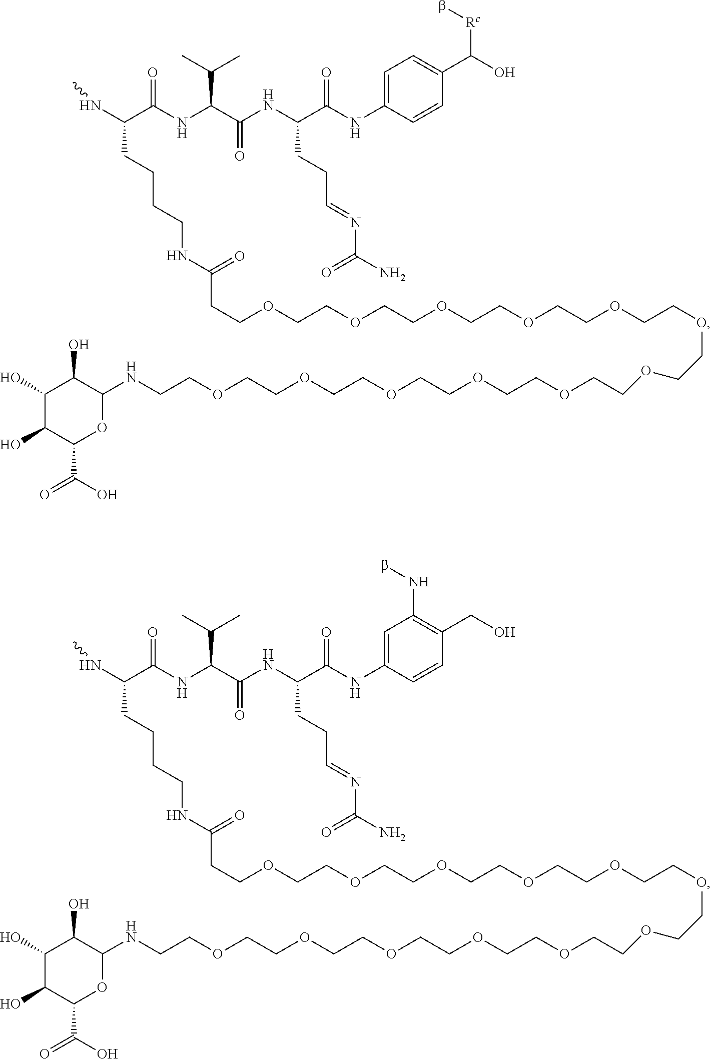

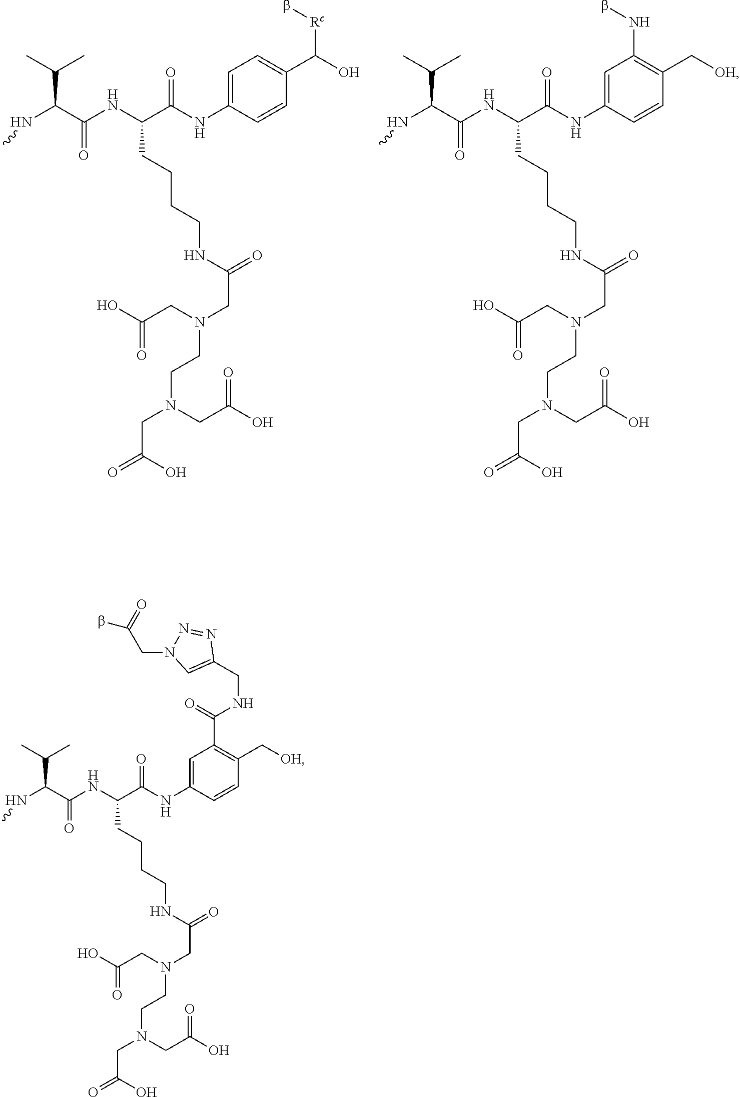

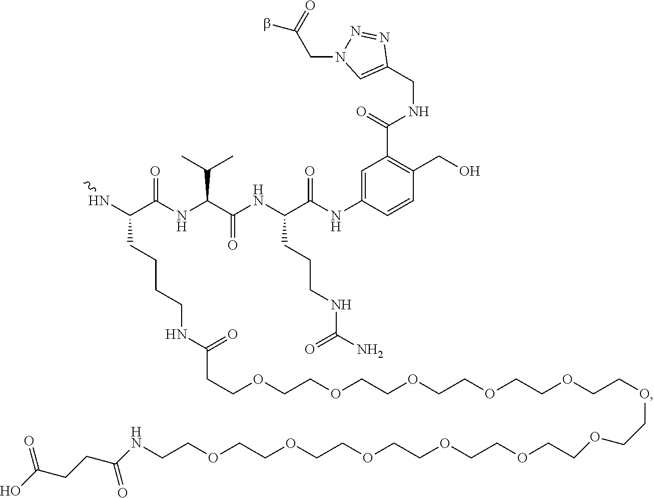

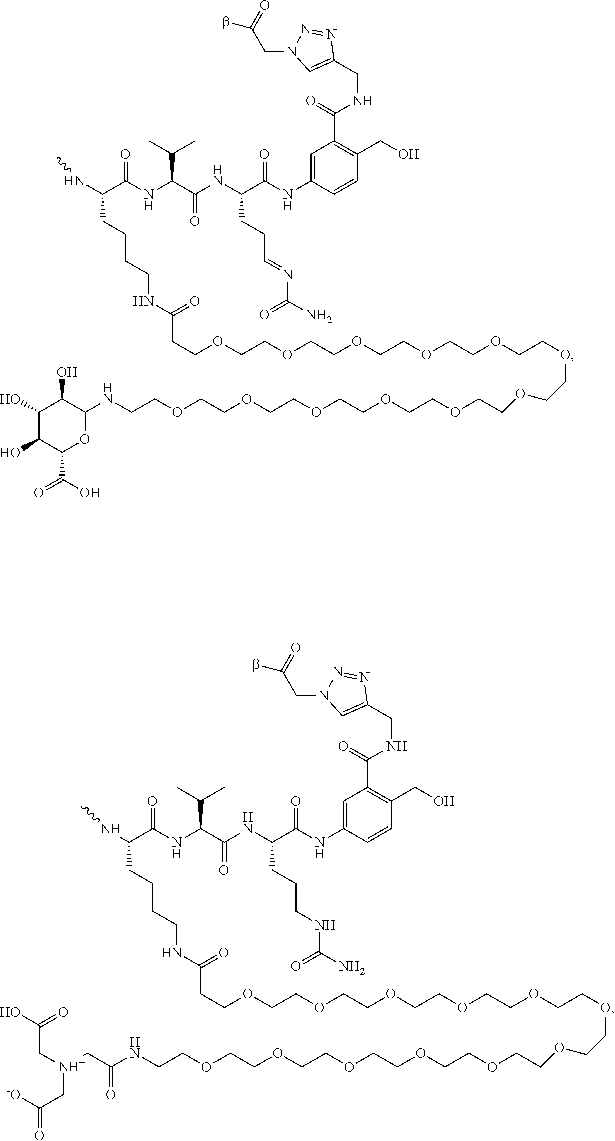

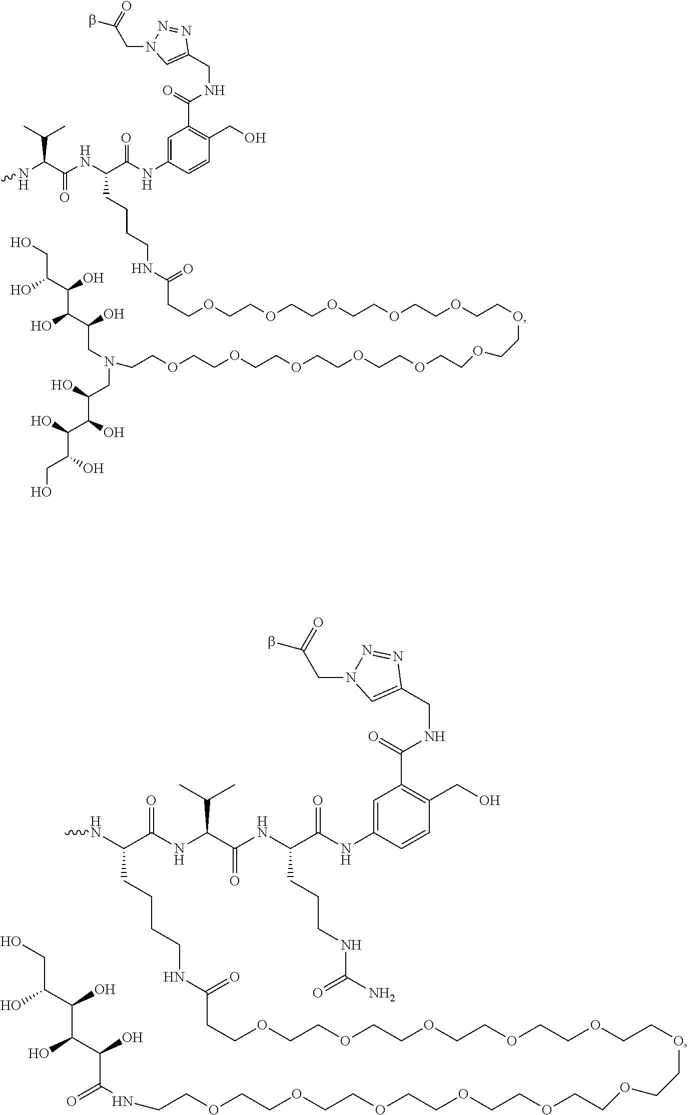

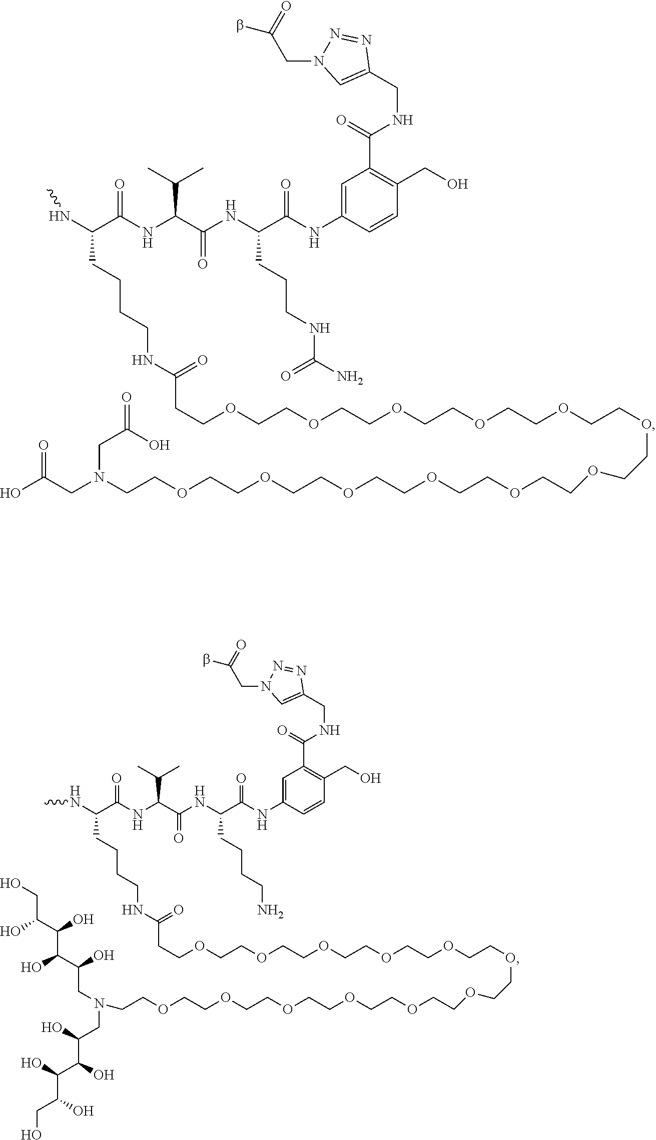

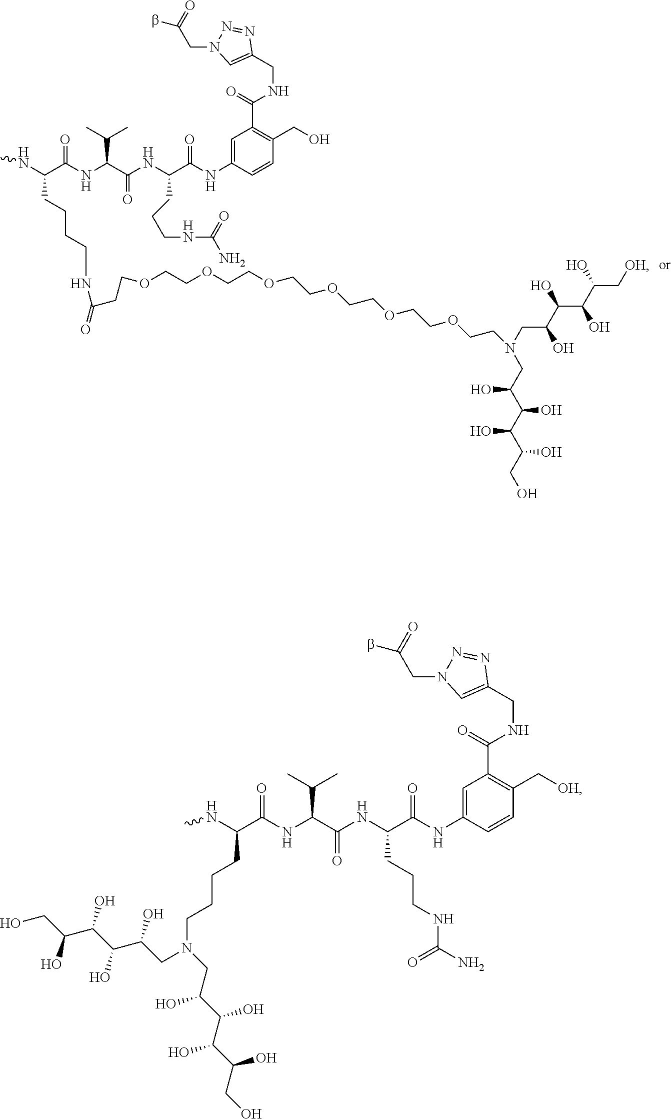

wherein: R^{c} is a bond or C₁₋₆ alkylene; the wavy line on the amino group indicates an attachment site for the Stretcher group; or, prior to attachment to the Stretcher group, indicates H; β— is an attachment site to the at least one Polar group; and the H on the benzylic OH is optionally replaced with a bond to at least one of the Drug units or to the attachment site to at least one of the Drug units.

In some embodiments, provided are Linker compounds having one of the following structures:

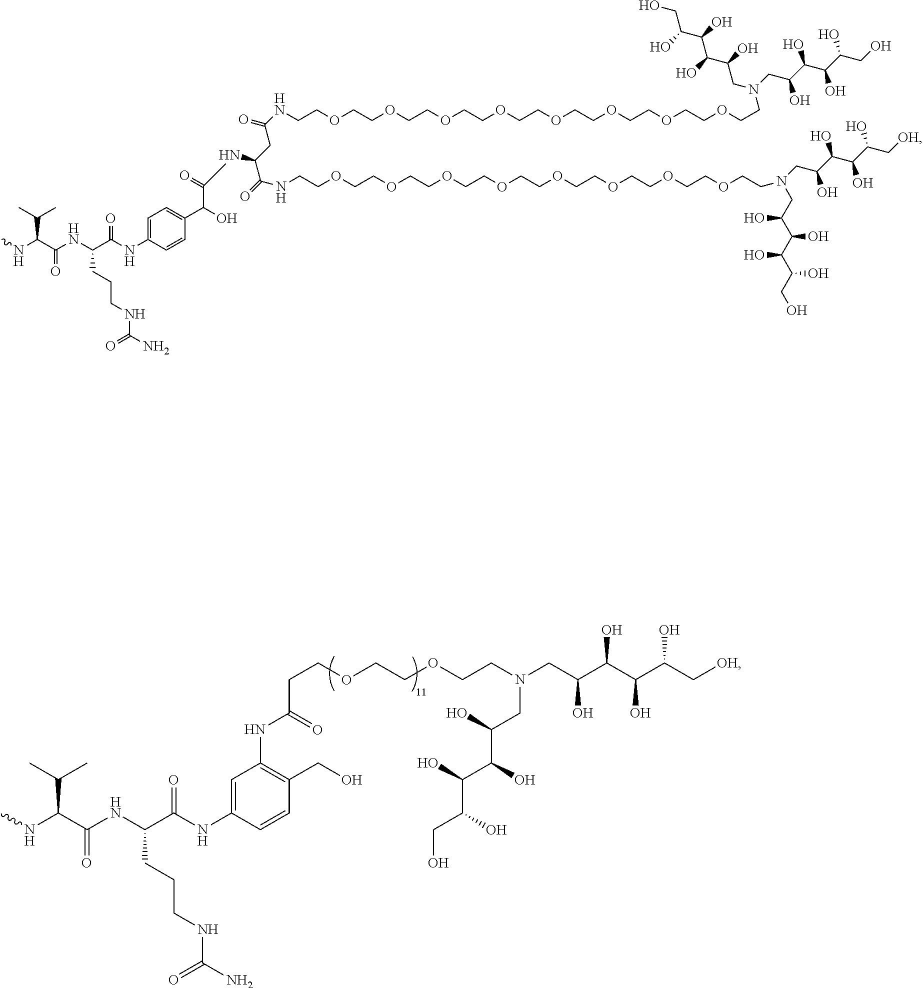

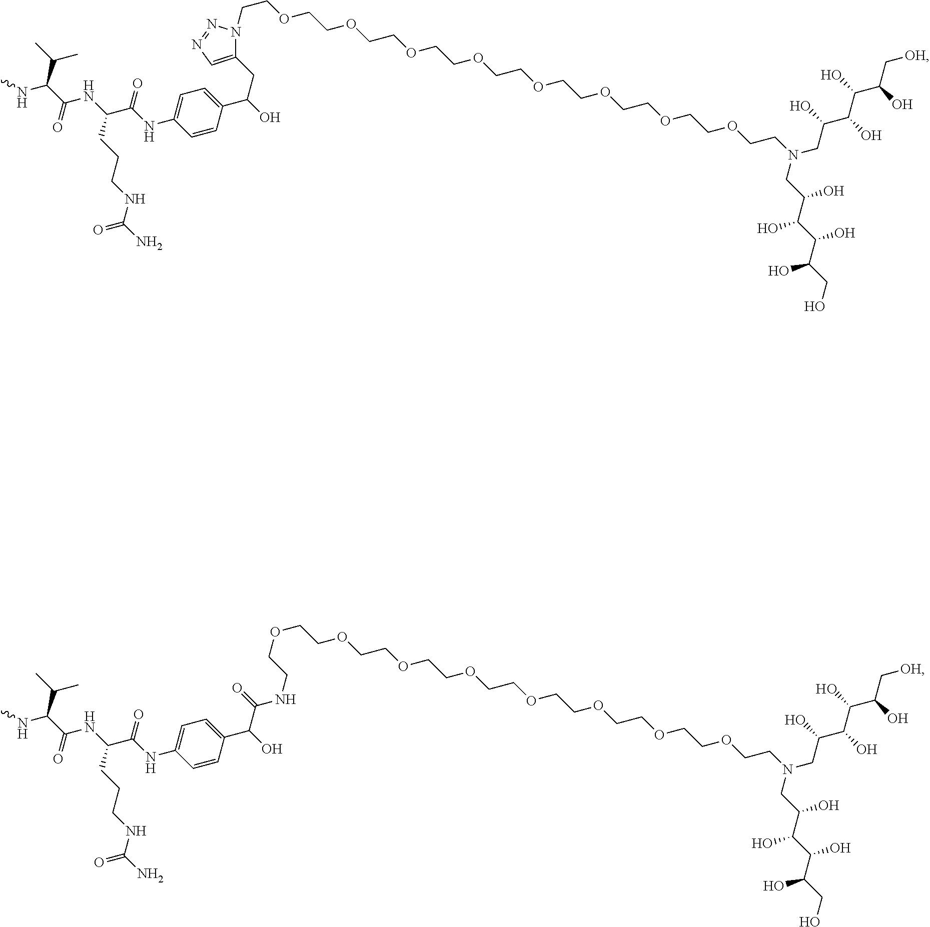

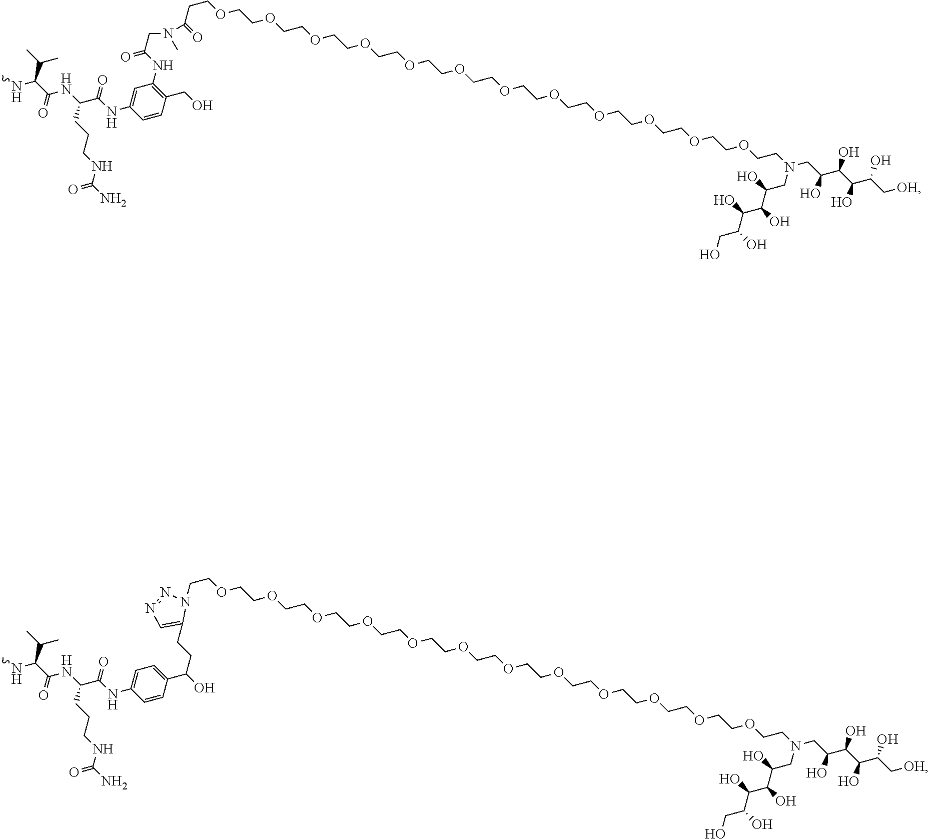

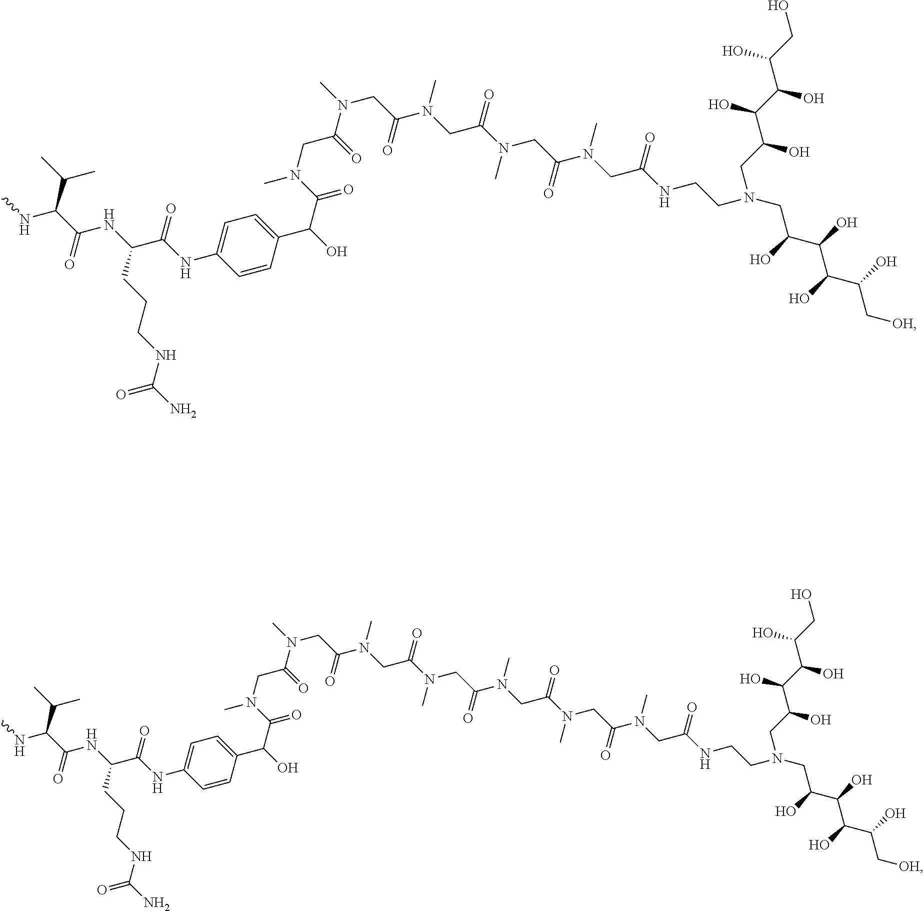

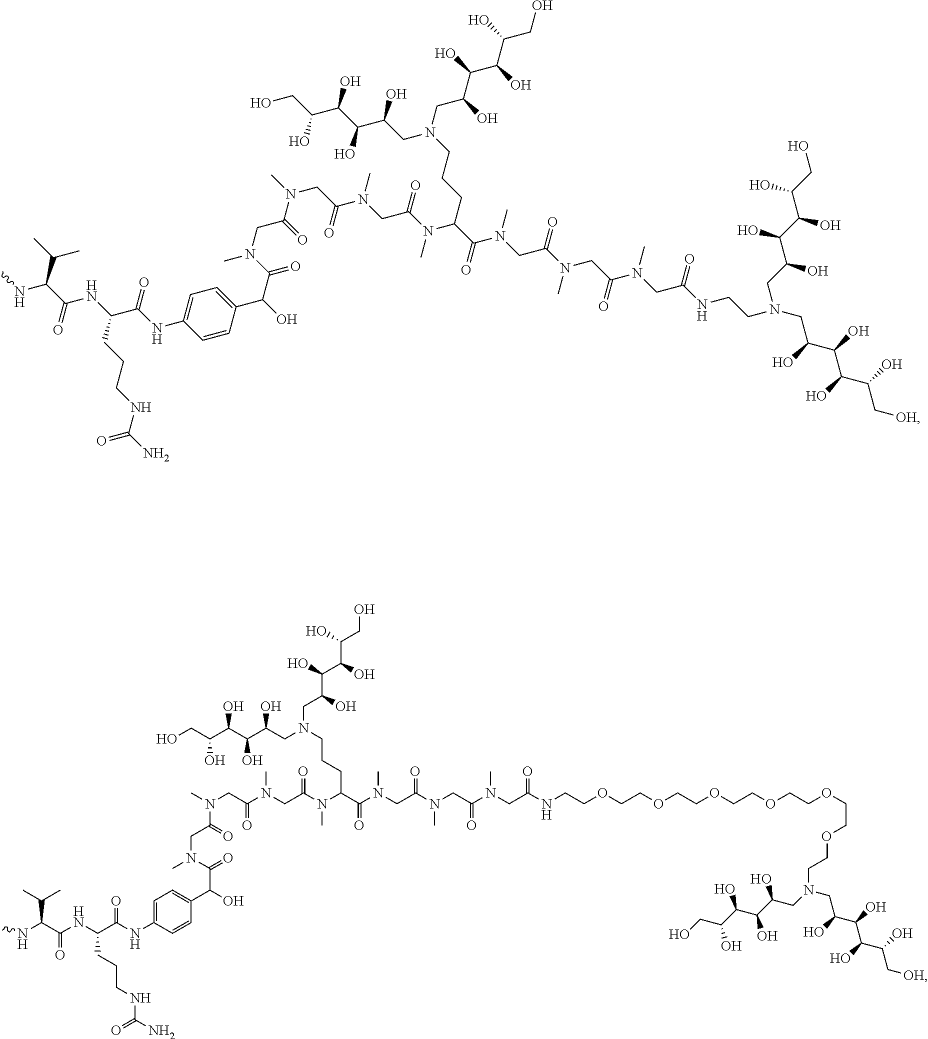

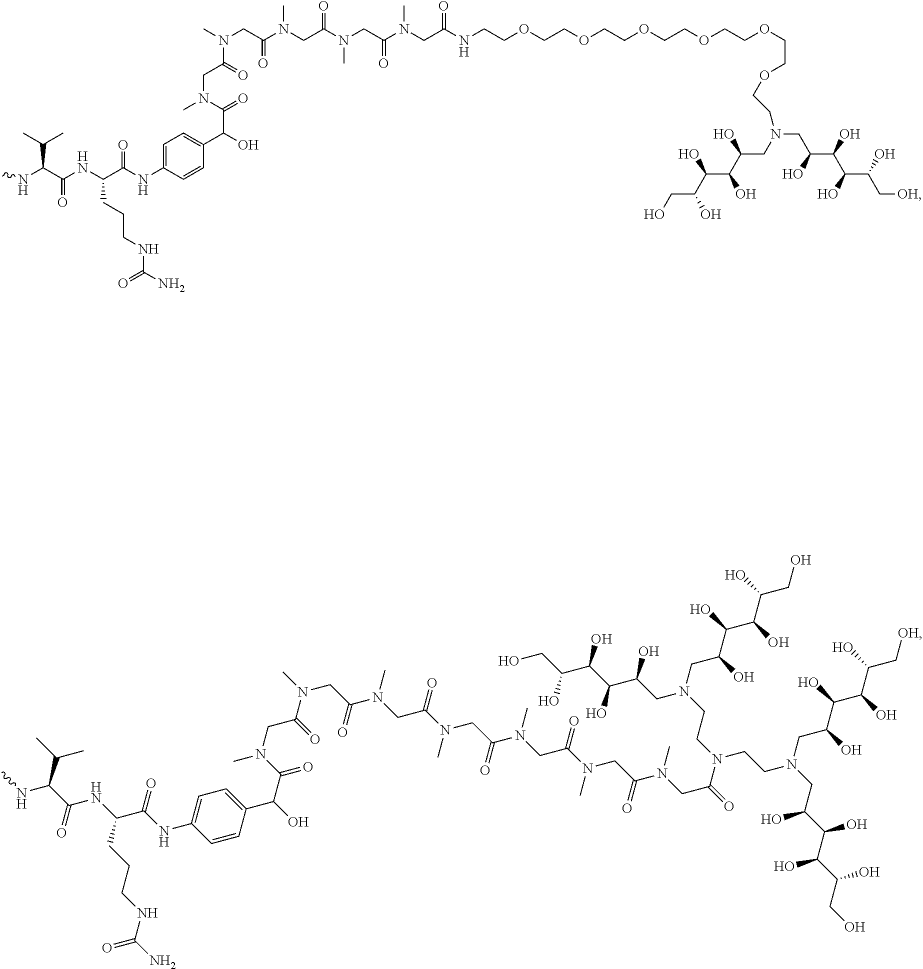

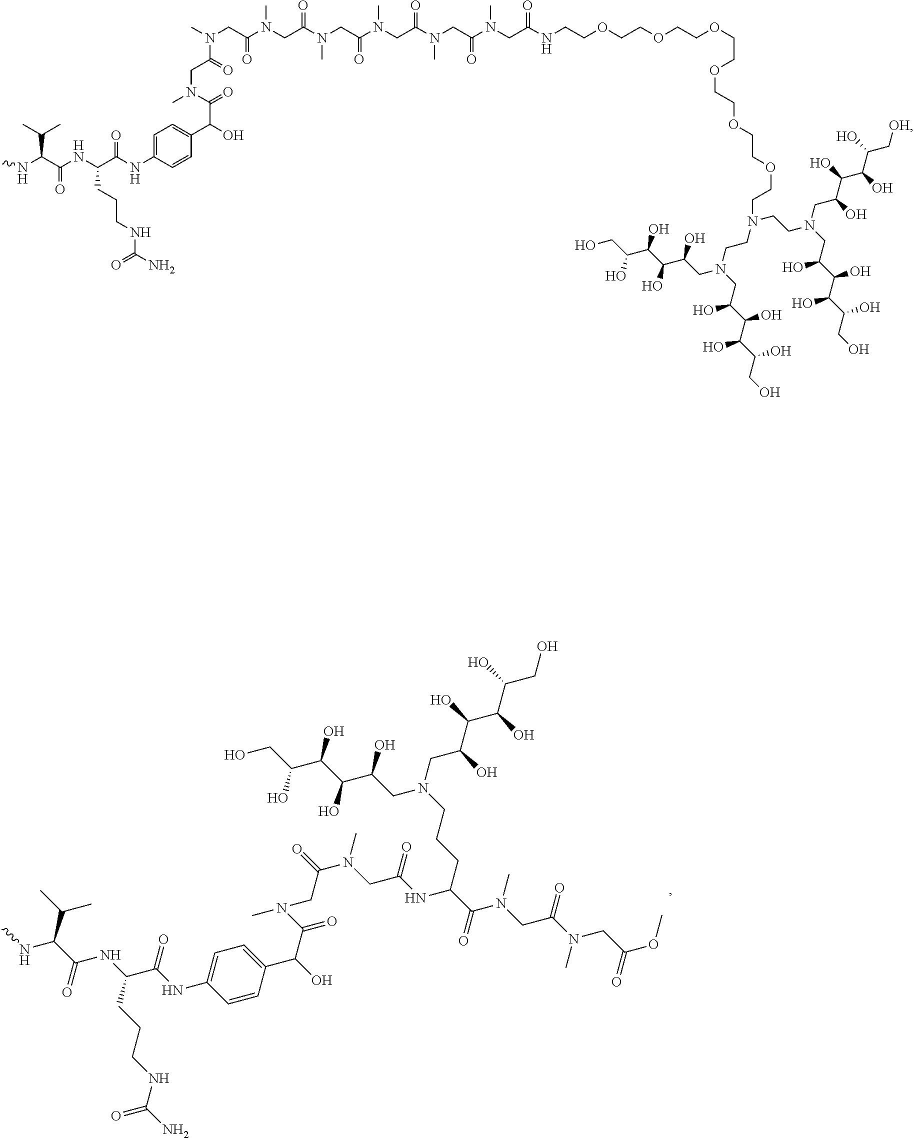

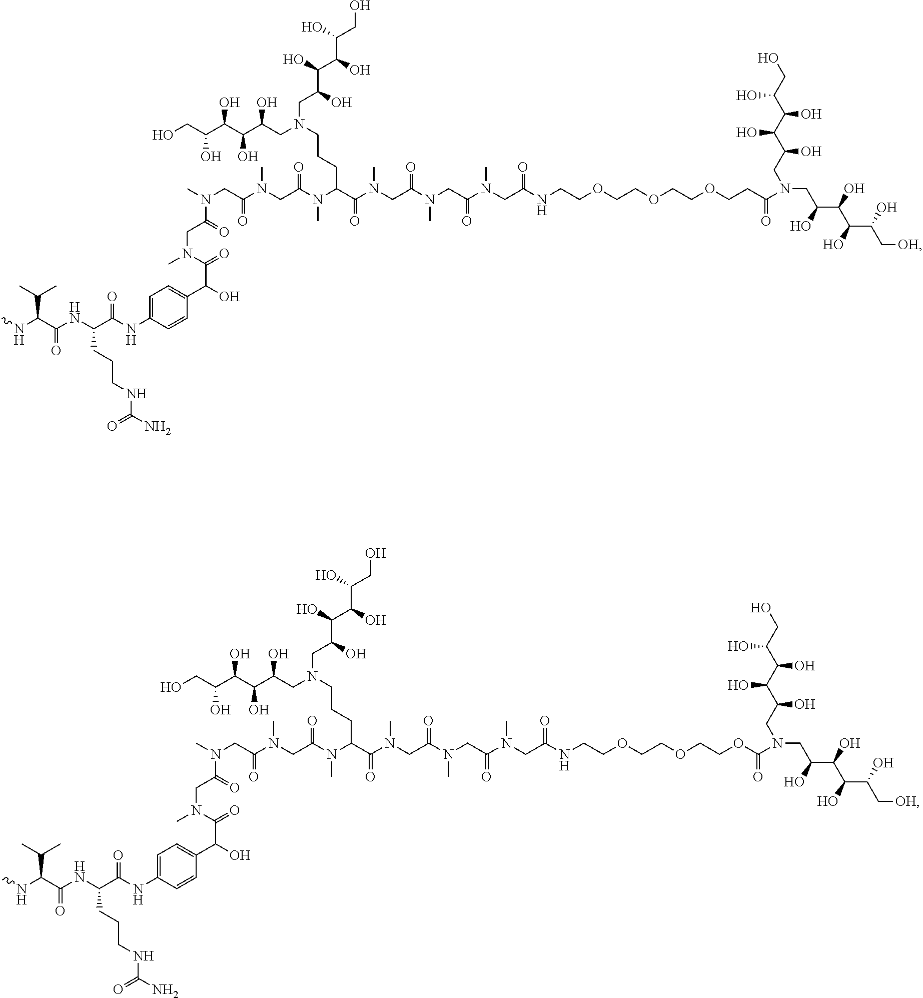

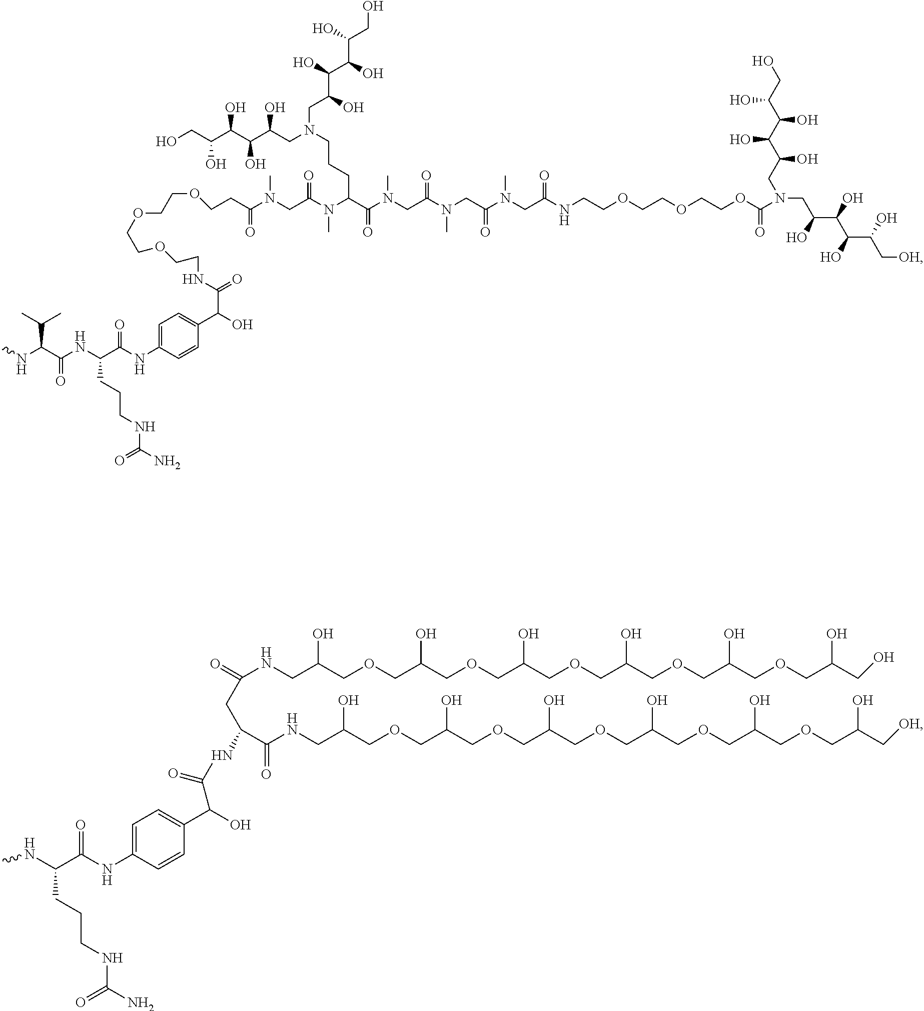

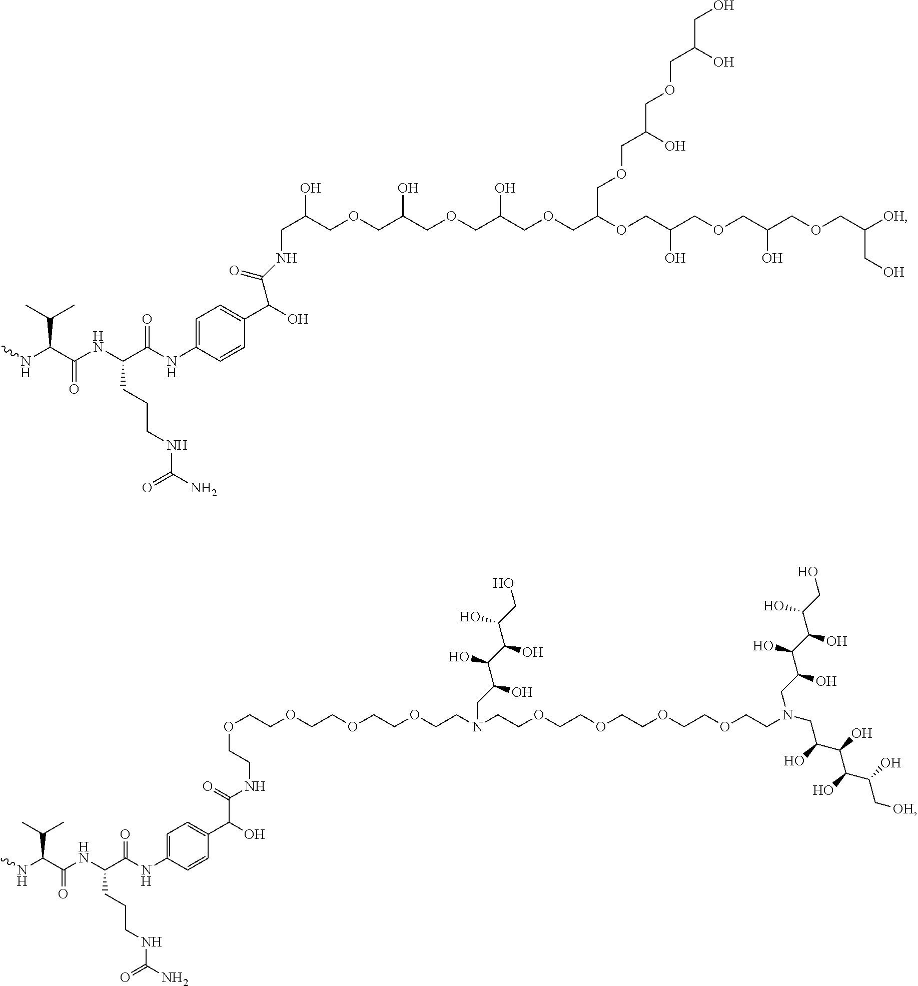

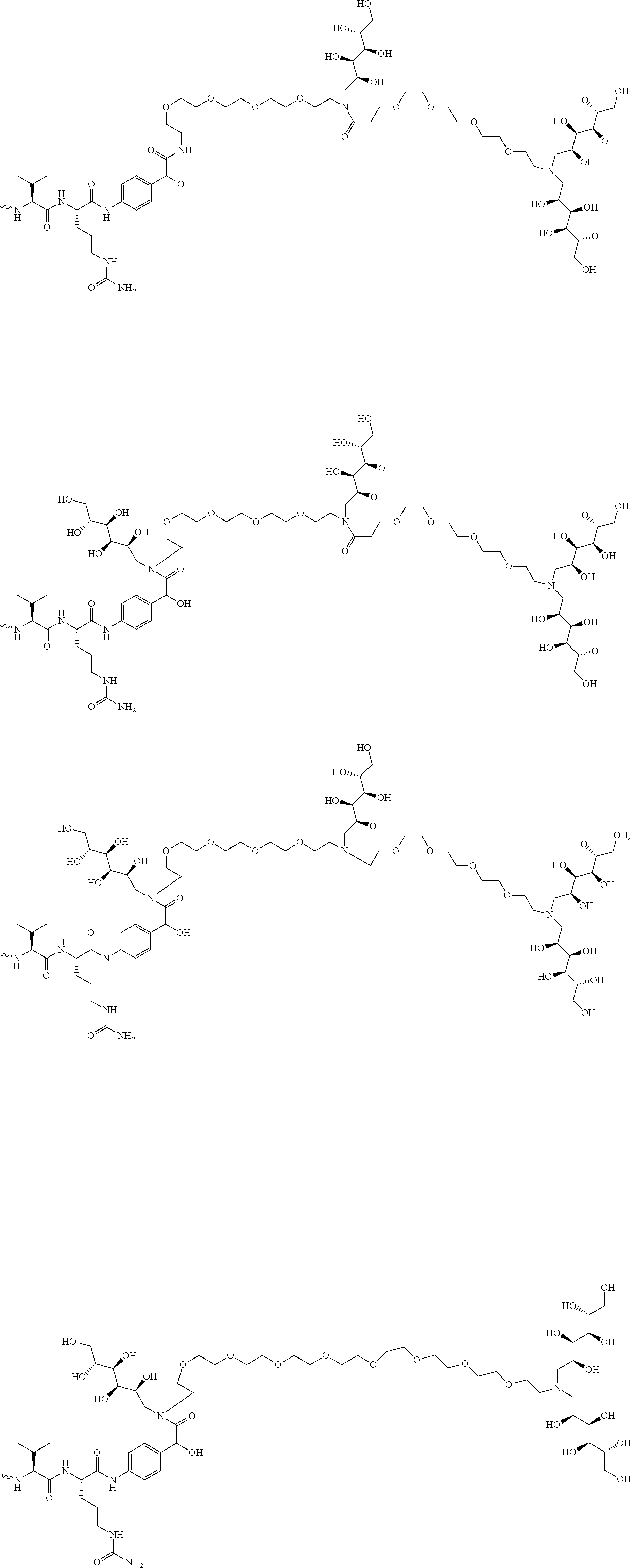

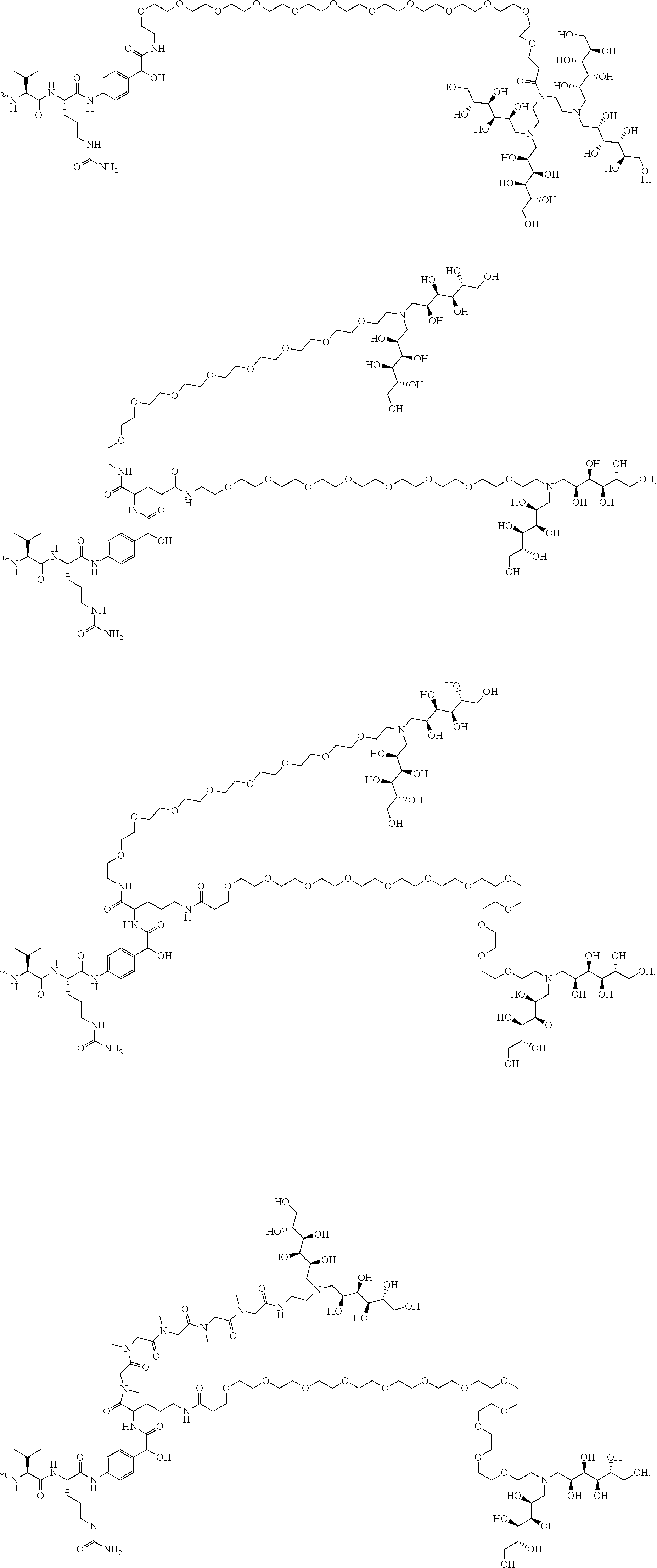

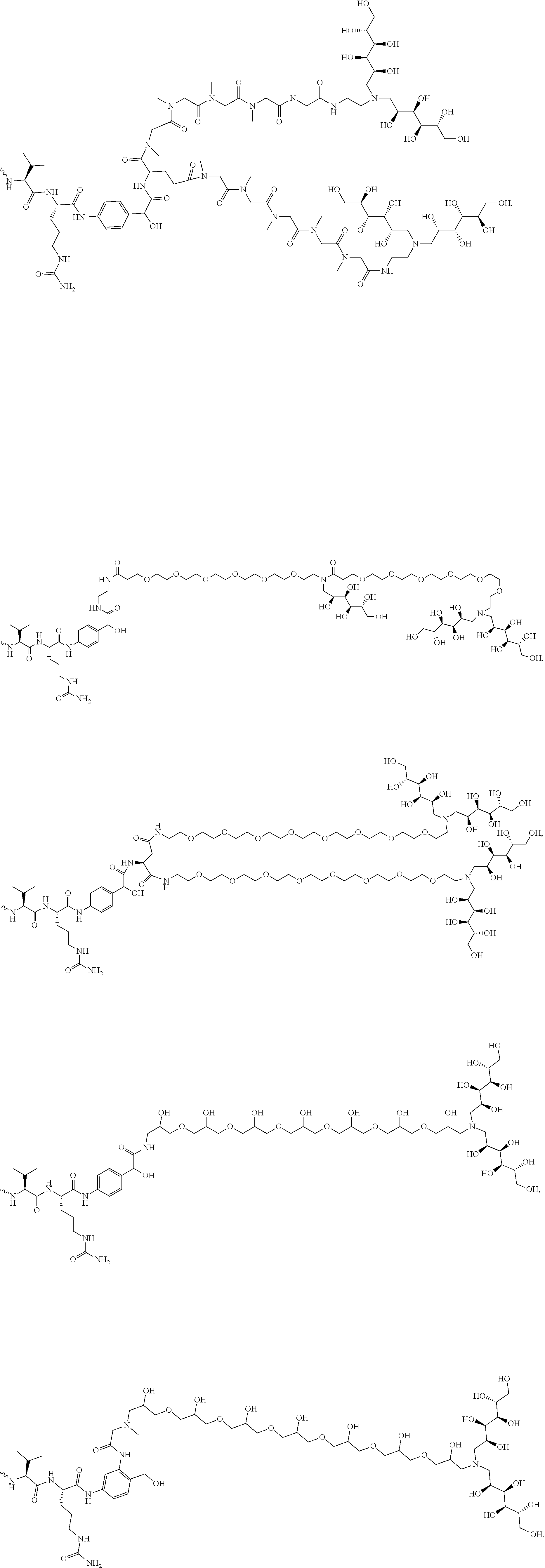

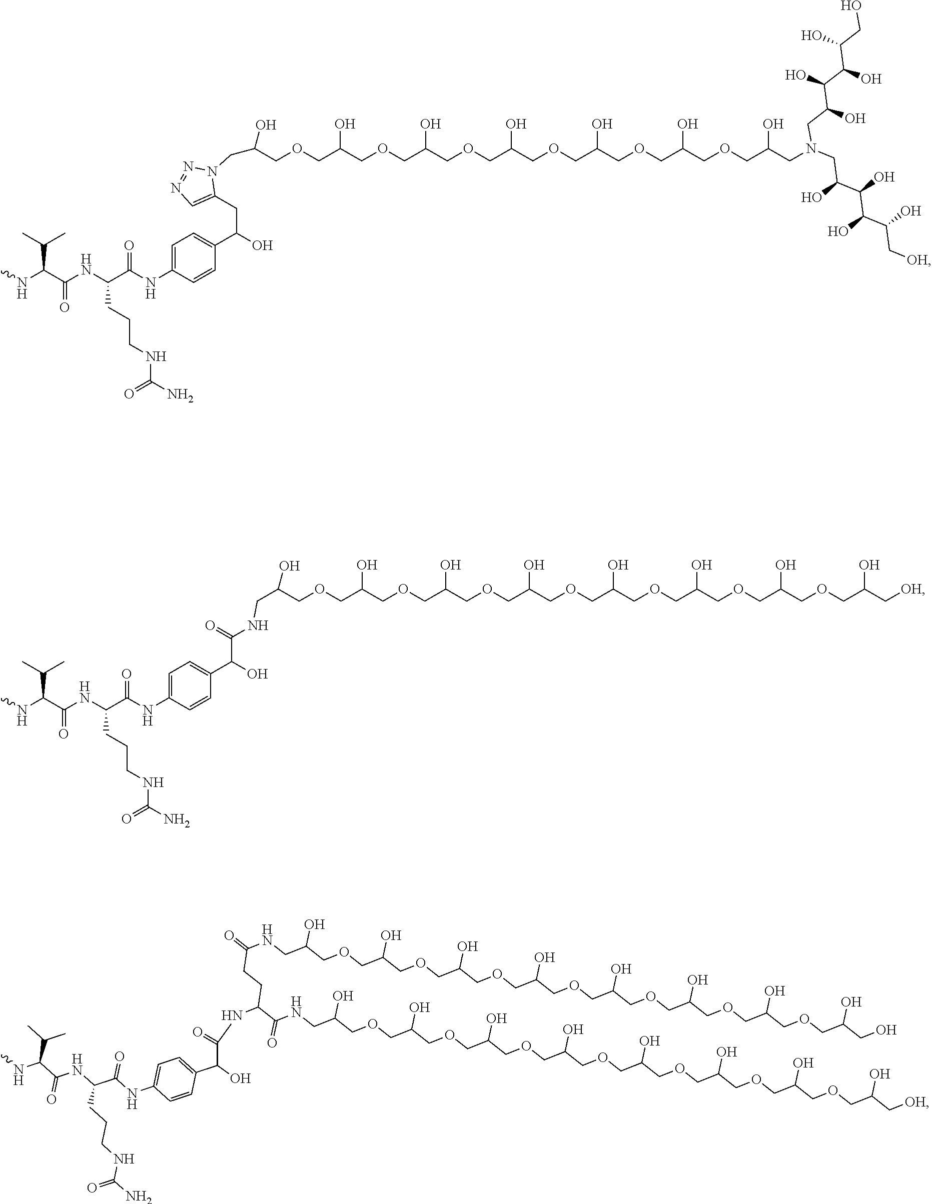

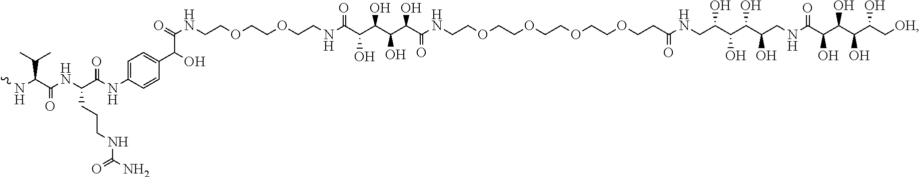

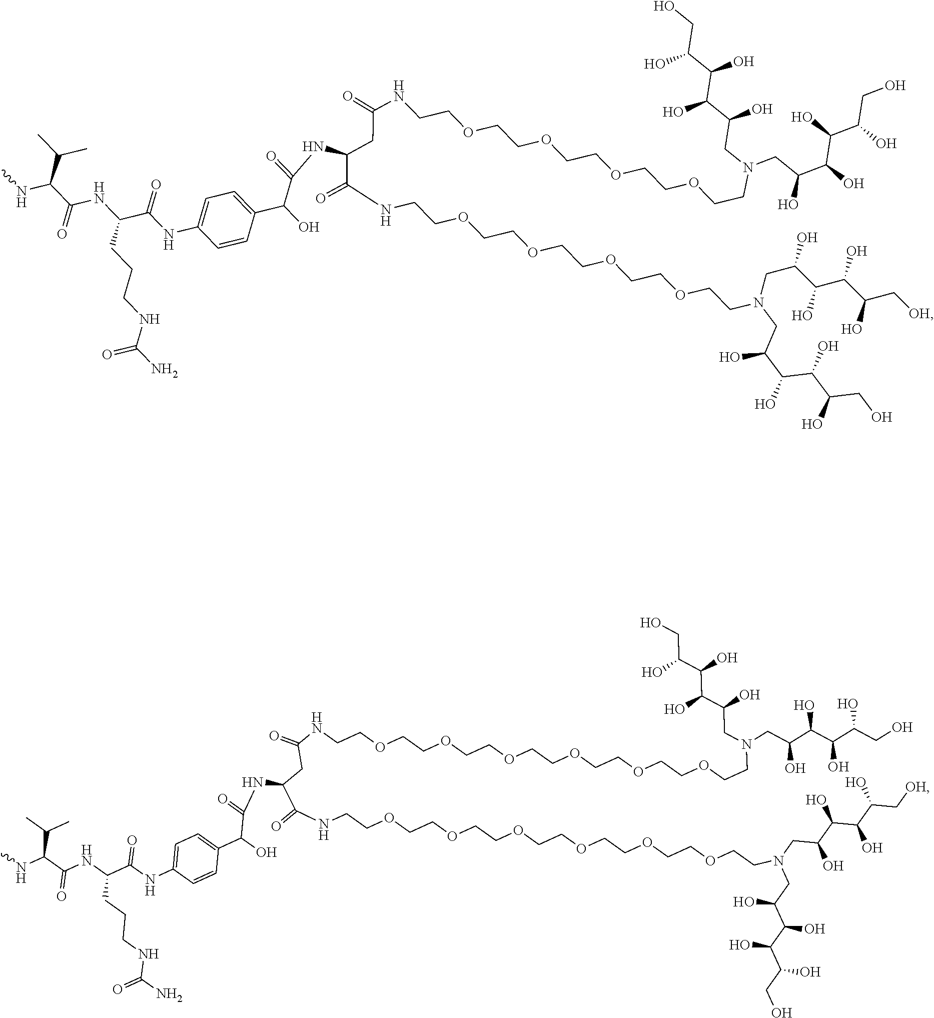

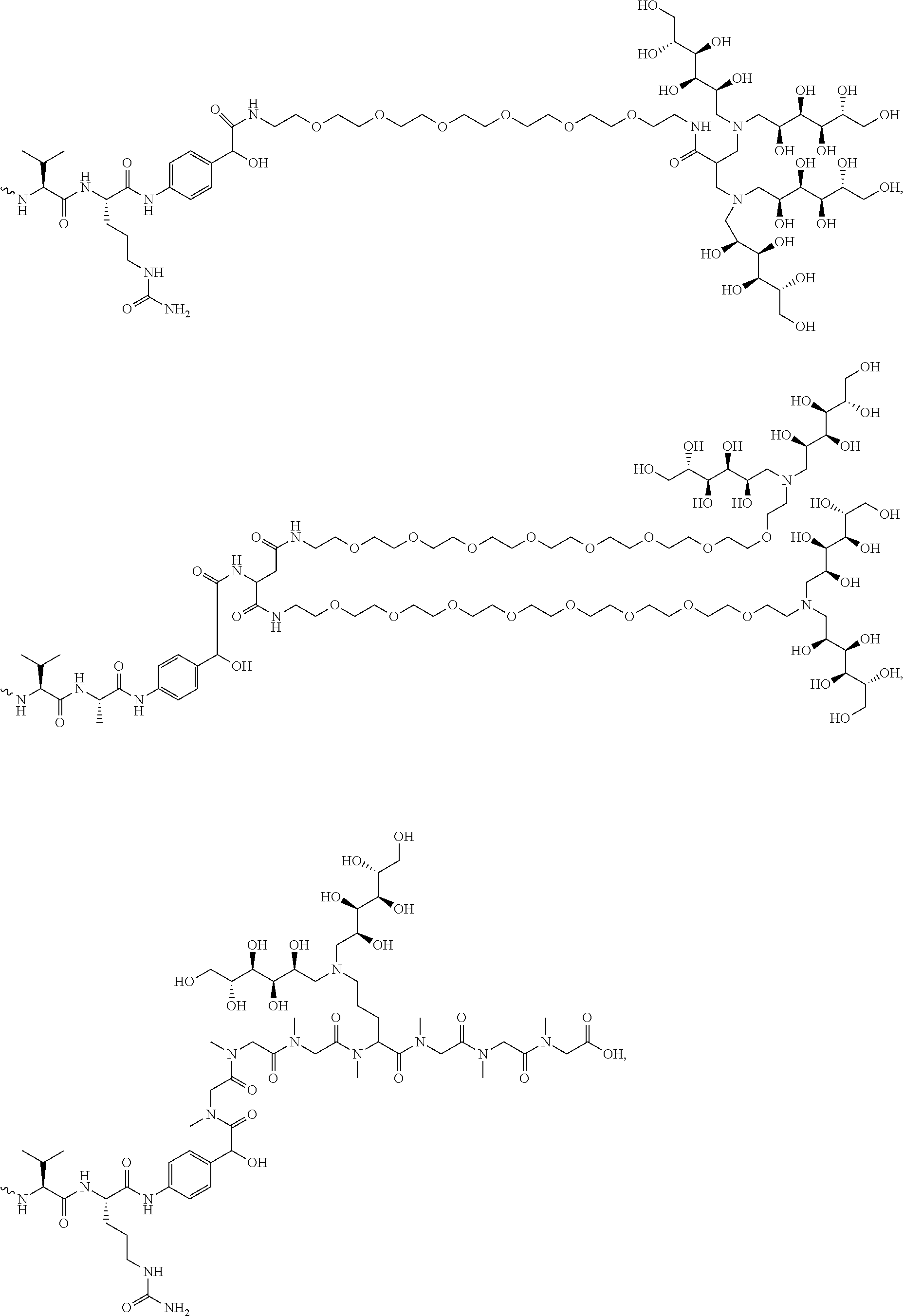

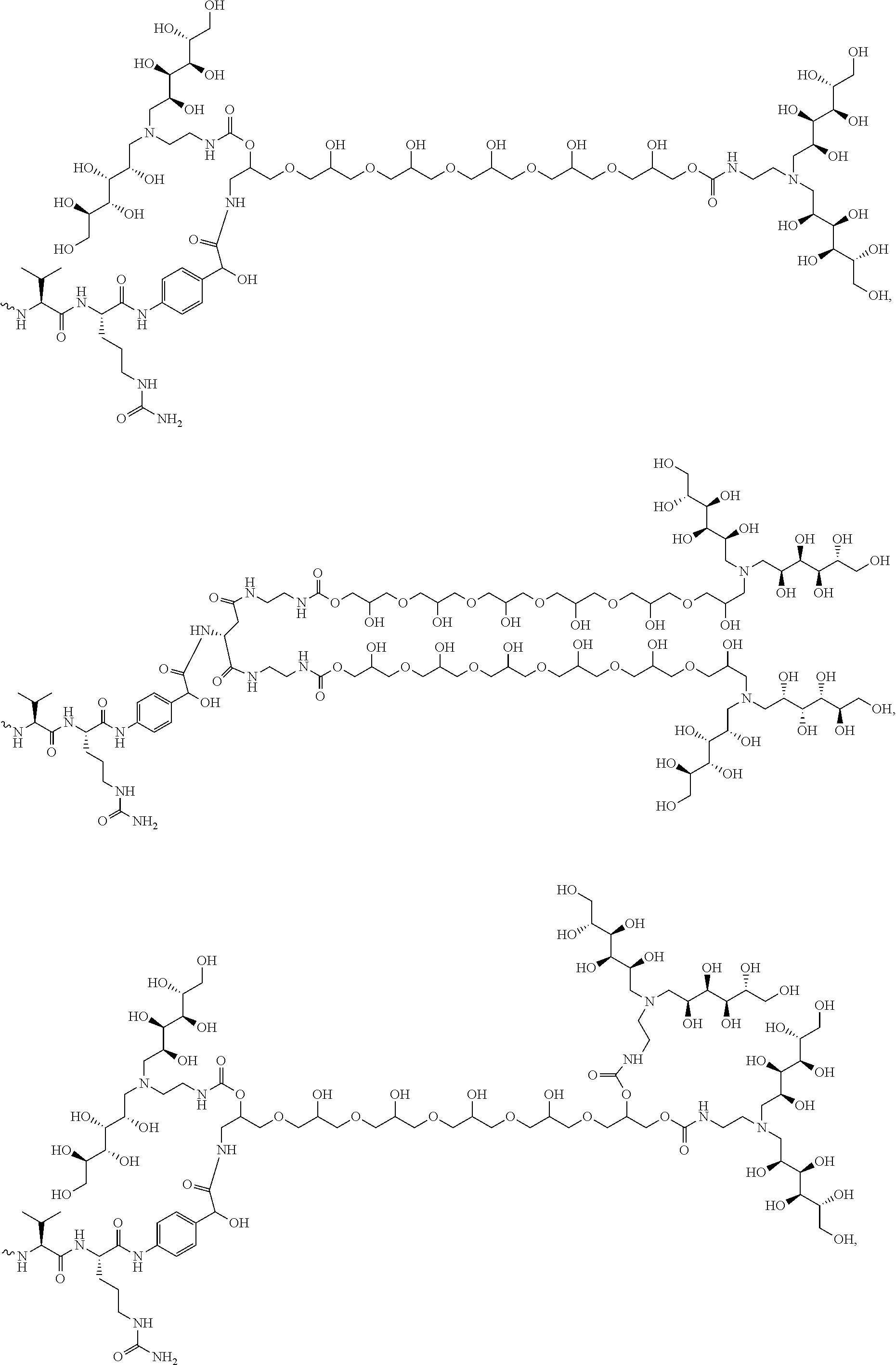

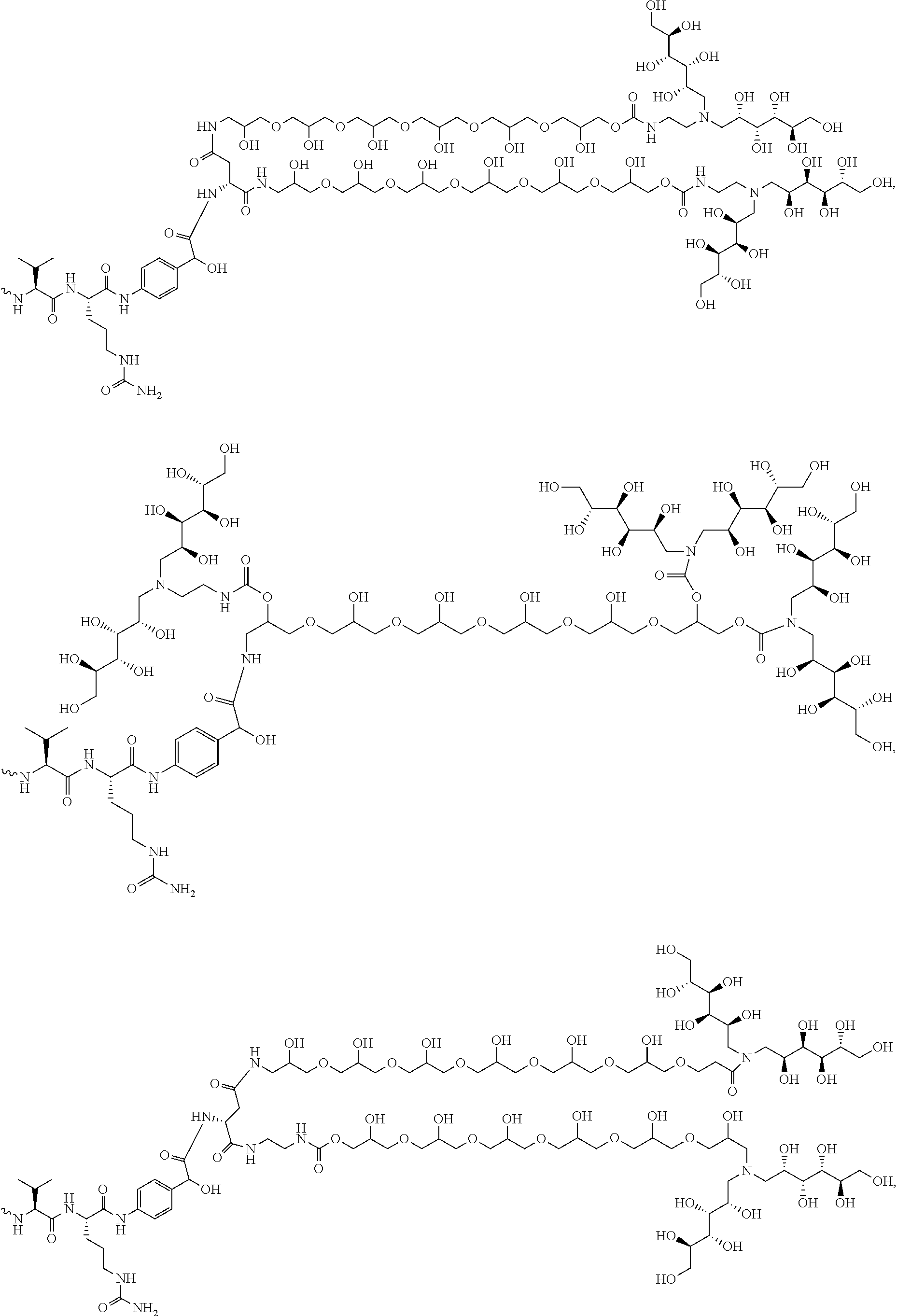

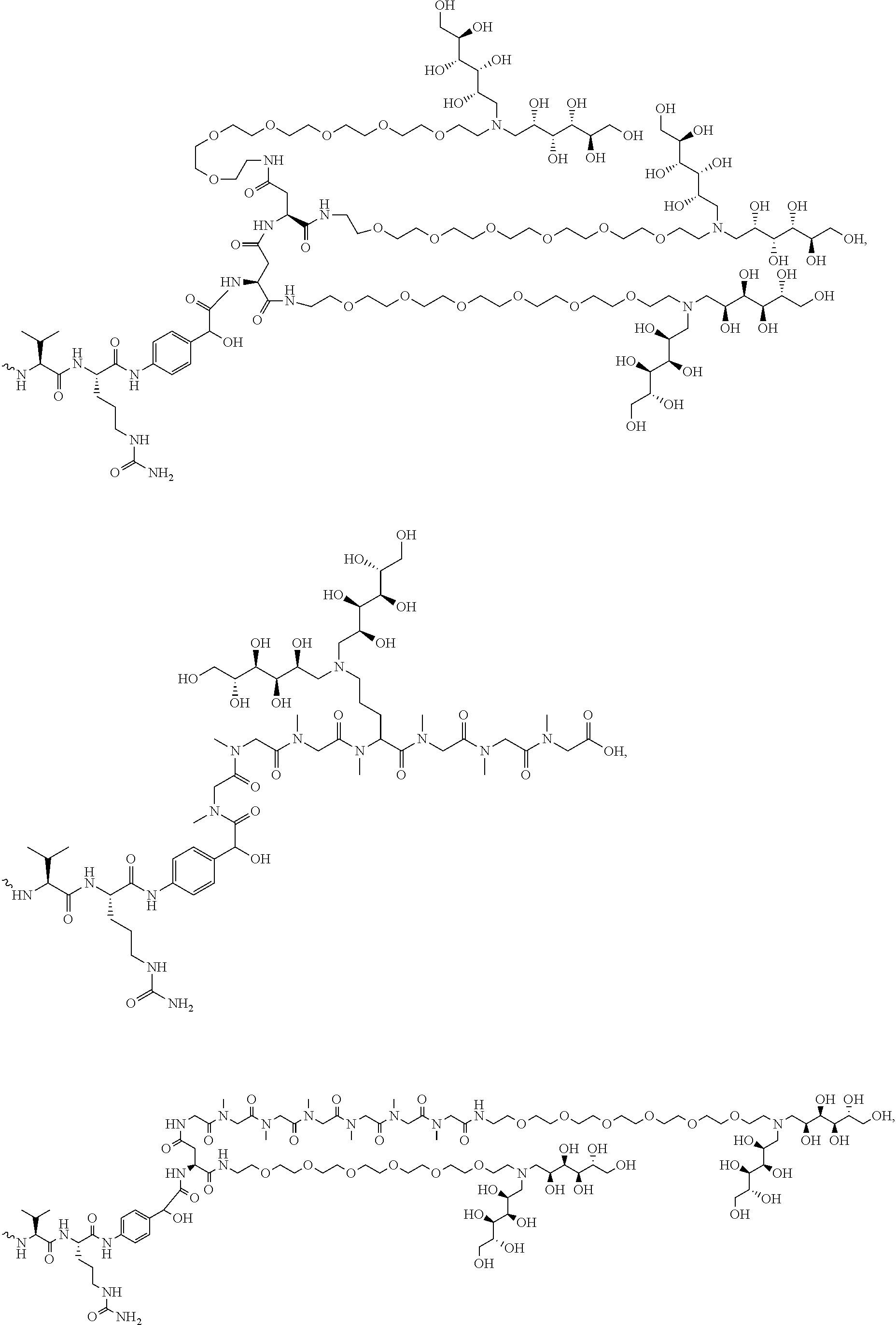

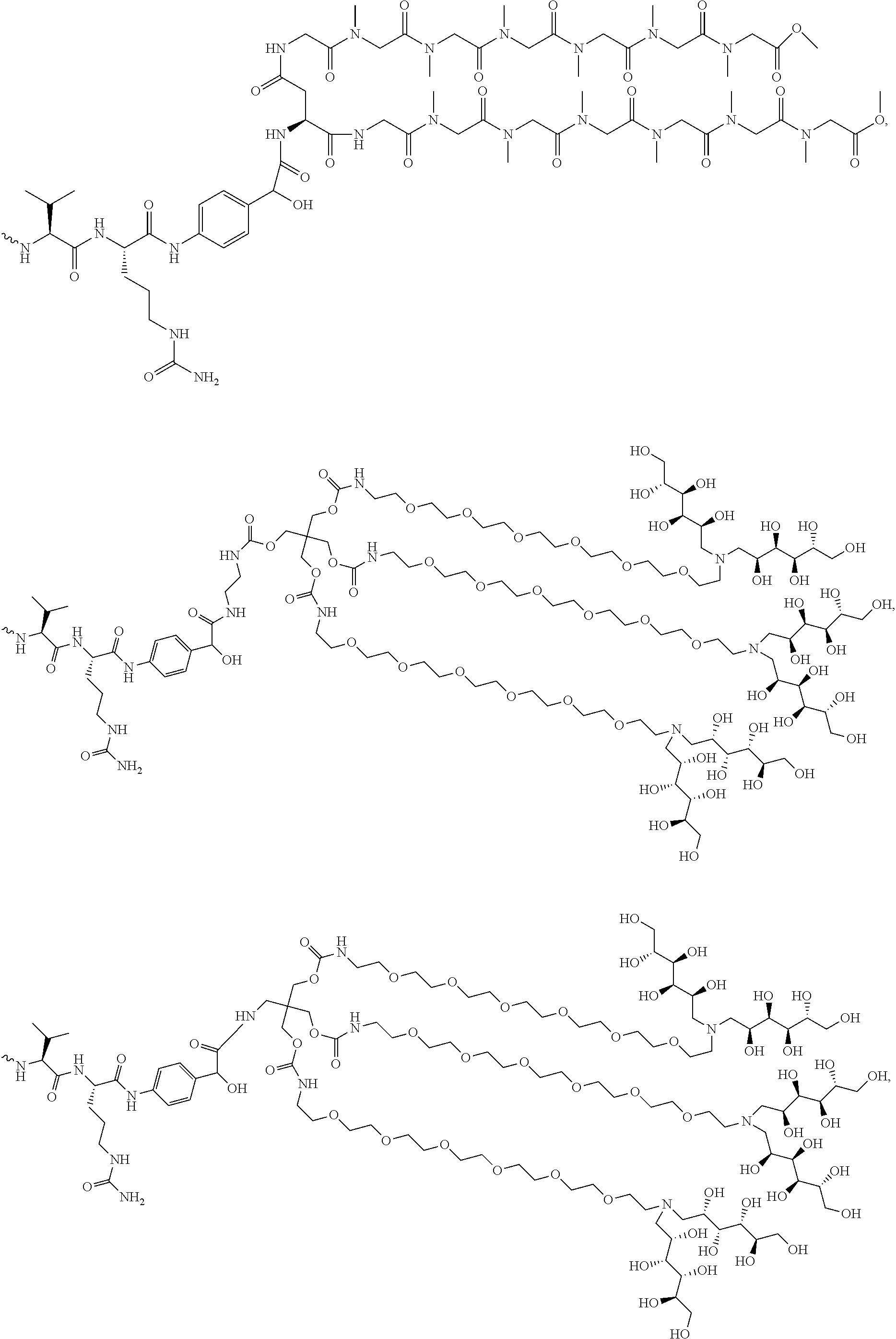

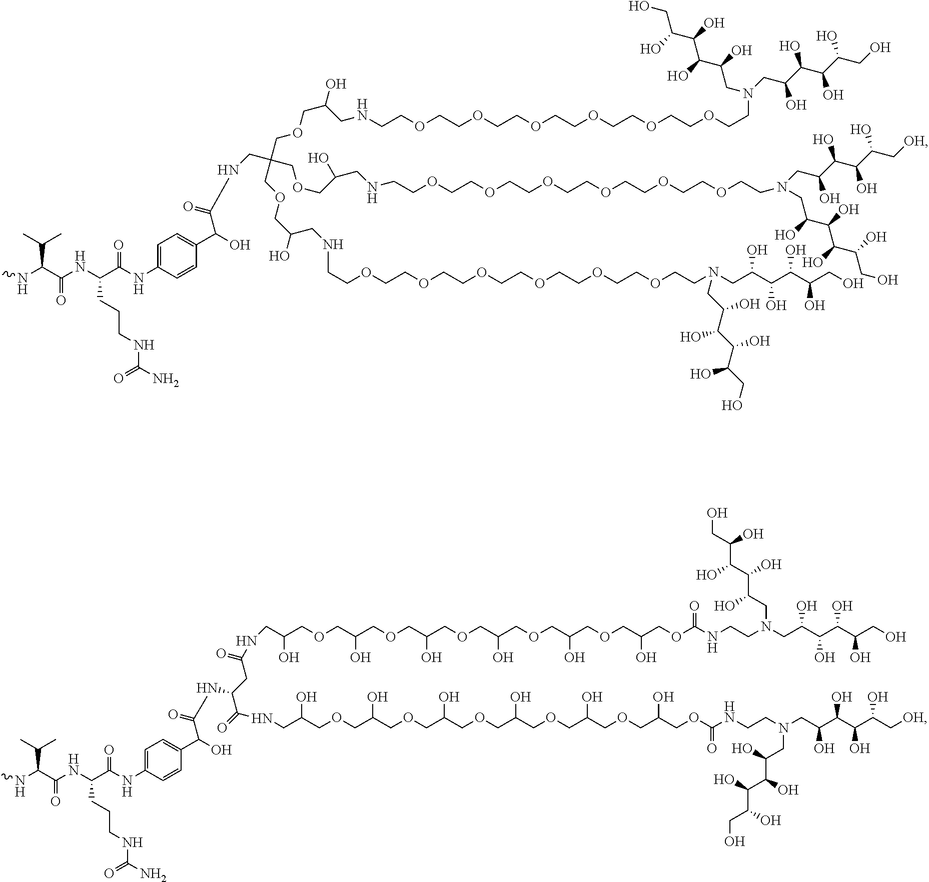

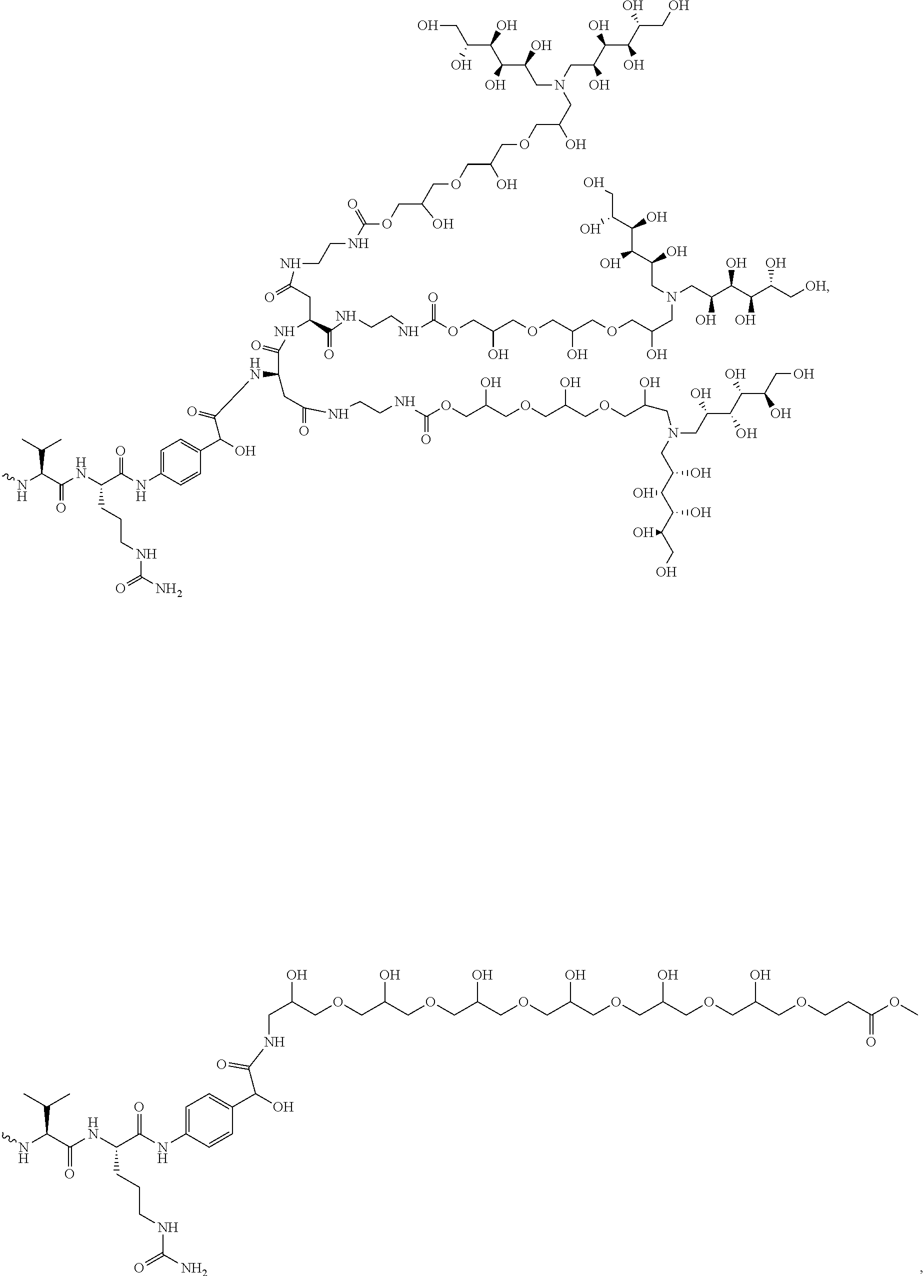

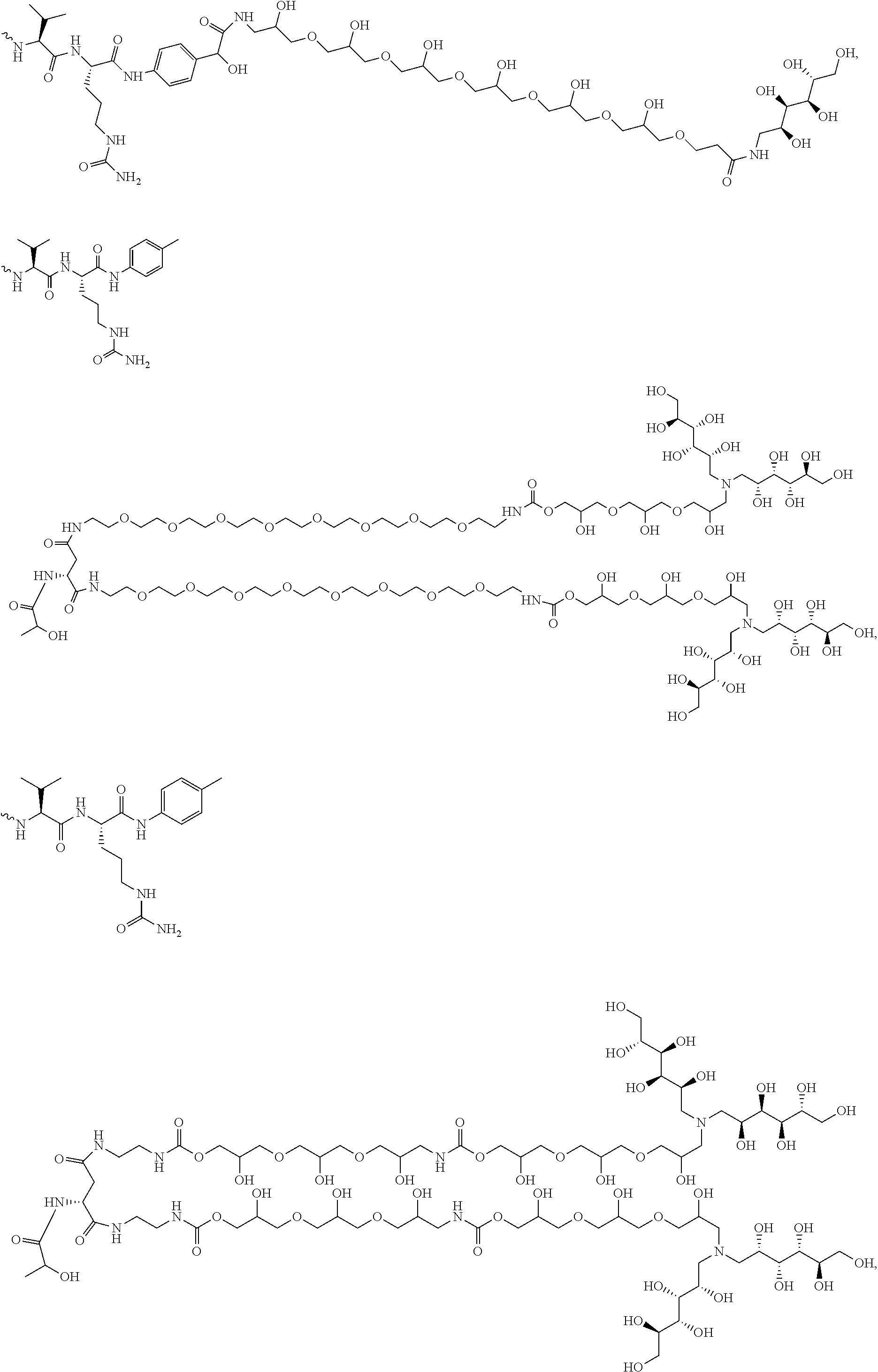

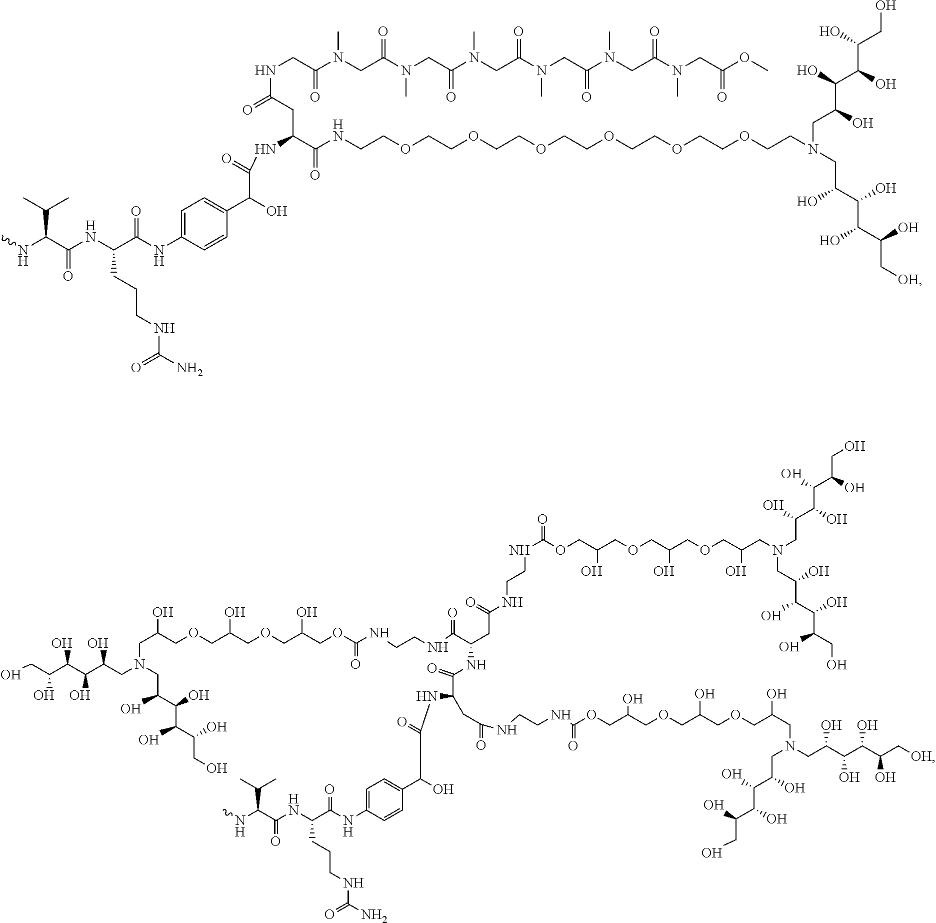

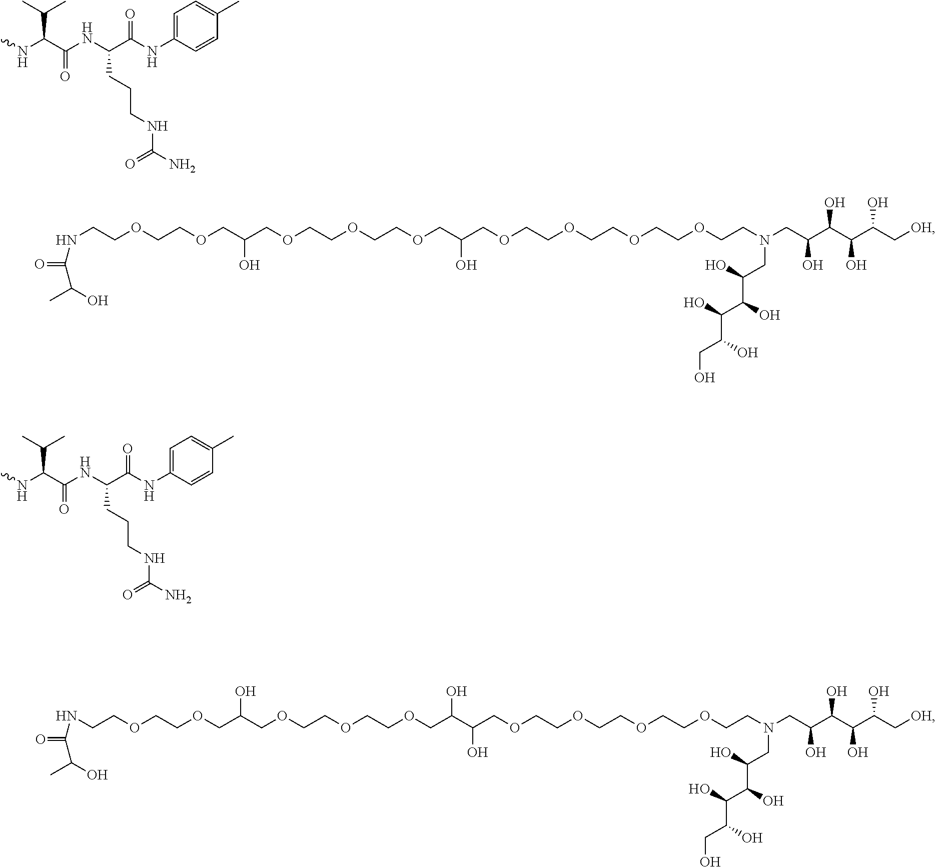

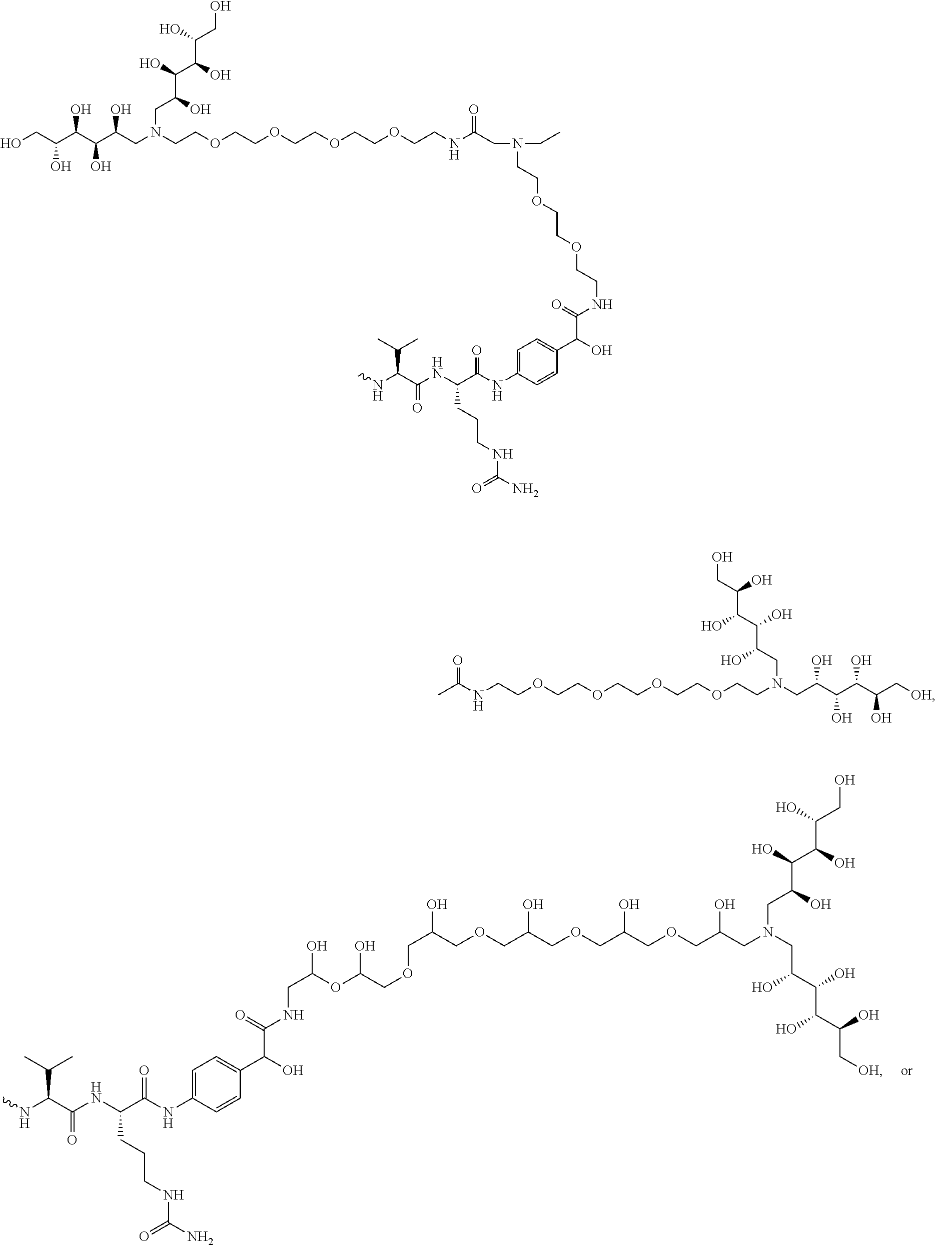

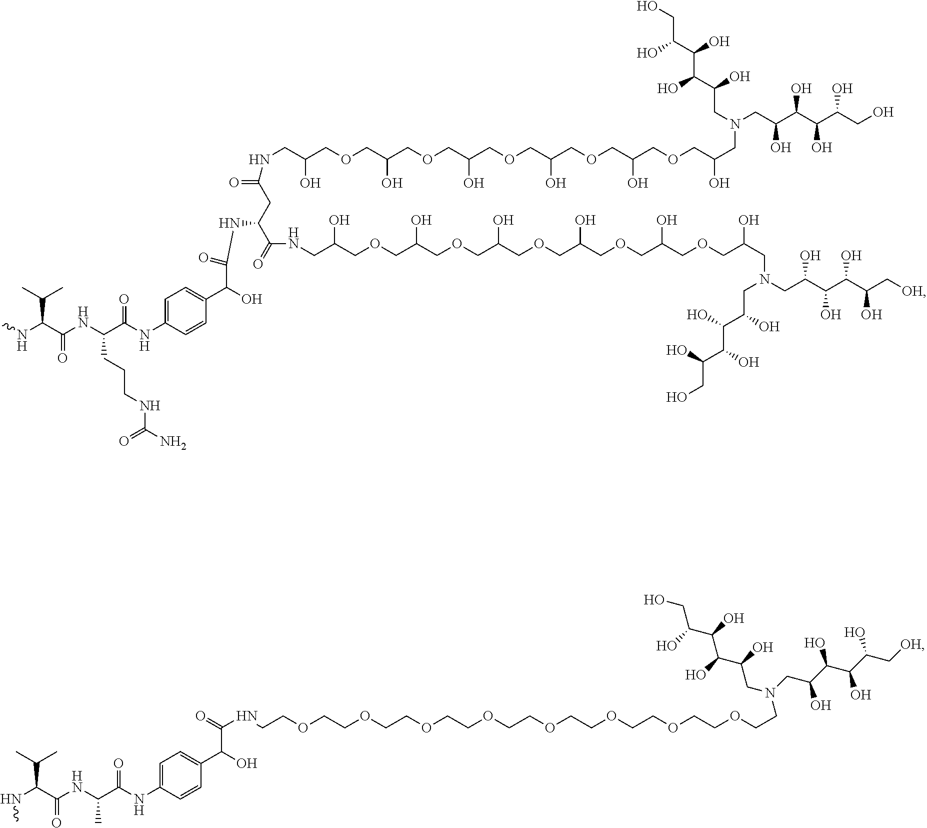

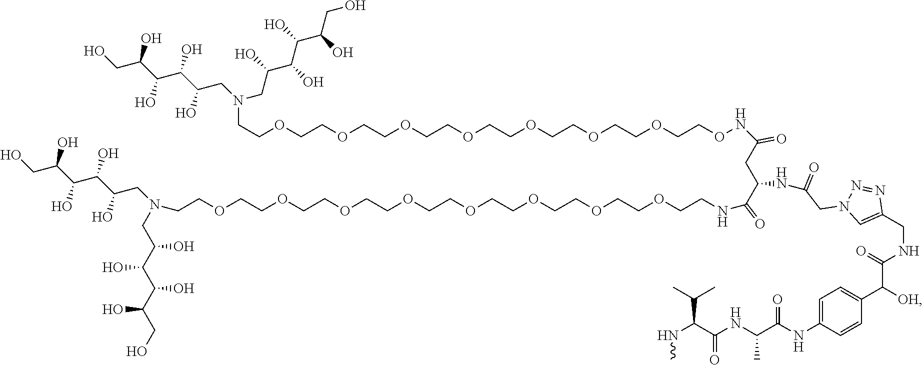

wherein the wavy line on the amino group indicates an attachment site to the Stretcher group; or, prior to attachment to the Stretcher group, indicates H, and the H on the benzylic OH is optionally replaced with a bond to at least one of the Drug units or a linking group attached to the at least one of the Drug units.

In some embodiments, provided are Linker compounds as described above wherein the Linker unit is attached to a side chain of a subunit of the enzyme-cleavable group.

In some embodiments, provided are Linker compounds a described above wherein the enzyme-cleavable group is joined to the Stretcher group by a non-peptidic linking group. For example, in some embodiments the non-peptidic linking group is selected from optionally-substituted C₁-C₁₀ alkylene, optionally-substituted C₂-C₁₀ alkenylene, optionally-substituted C₂-C₁₀ alkynylene, or optionally-substituted polyethylene glycol.

In some embodiments, provided are Linker compounds as described above comprising the Stretcher group attached to the enzyme-cleavable group.

### Stretcher Groups

In some embodiments, provided are Linker compounds comprising a Stretcher group is selected from the following:

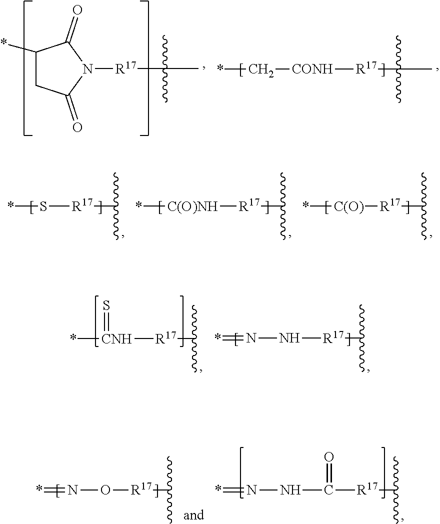

wherein R¹⁷ is —C₁-C₁₀ alkylene-, —C₁-C₁₀ heteroalkylene-, —C₃-C₈ carbocyclo-, —O—(C₁-C₈ alkylene)-, —(CH₂—O—CH₂)_{b}—C₁-C₈ alkylene- (where b is 1 to 26), —C₁-C₈ alkylene-(CH₂—O—CH₂)_{b}— (where b is 1 to 26), —C₁-C₈ alkylene-(CH₂—O—CH₂)_{b}—C₁-C₈ alkylene- (where b is 1 to 26), -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, —C₁-C₁₀alkylene-C(O)NH—C₁-C₈alkylene-[O—CH₂—CH₂]ₙ—C(O)— (where n is 1 to 26), C₁-C₁₀ heteroalkylene-C(═O)—, —C₁-C₈ alkylene-(CH₂—O—CH₂)_{b}—C(═O)— (where b is 1 to 26), —(CH₂—O—CH₂)_{b}—C₁-C₈ alkylene-C(═O)— (where b is 1 to 26), —C₁-C₈ alkylene-(CH₂—O—CH₂)_{b}—C₁-C₈ alkylene-C(═O)— (where b is 1 to 26), —C₃-C₈ carbocyclo-C(═O)—, —O—(C₁-C₈ alkyl)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, —C₁-C₁₀ heteroalkylene-NH—, —C₁-C₈ alkylene-(CH₂—O—CH₂)_{b}—NH— (where b is 1 to 26), —(CH₂—O—CH₂)_{b}—C₁-C₈ alkylene-NH— (where b is 1 to 26), —C₁-C₈ alkylene-(CH₂—O—CH₂)_{b}—C₁-C₈ alkylene-NH— (where b is 1 to 26), —C₁-C₈ alkylene-(C(═O))—NH—(CH₂—O—CH₂)_{b}—C(═O)— (where b is 1 to 26), —C₁-C₈ alkylene-(C(═O))—NH—(CH₂—O—CH₂)_{b}—C₁-C₈ alkylene-C(═O)— (where b is 1 to 26), —C₁-C₈ alkylene-NH—(C(═O))—(CH₂—O—CH₂)_{b}—NH— (where b is 1 to 26), —C₁-C₈ alkylene-NH—(C(═O))—(CH₂—O—CH₂)_{b}—C₁-C₈ alkylene-NH— (where b is 1 to 26), —C₃-C₈ carbocyclo-NH—, —O—(C₁-C₈ alkyl)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkyl)-S—, -arylene-S—, —C₁-C₁(alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—; or wherein the Stretcher group comprises maleimido(C₁-C₁₀alkylene-C(O)—, maleimido(CH₂OCH₂)p₂(C₁-C₁₀ alkyene)C(O)—, maleimido(C₁-C₁₀ alkyene) (CH₂OCH₂)ₚ₂C(O)—, or a ring open form thereof, wherein p2 is from 1 to 26; and wherein * is an attachment site to the Targeting group, and the wavy line is an attachment site to the enzyme-cleavable group.

In some embodiments, provided are Linker compounds comprising a Stretcher group is selected from the following:

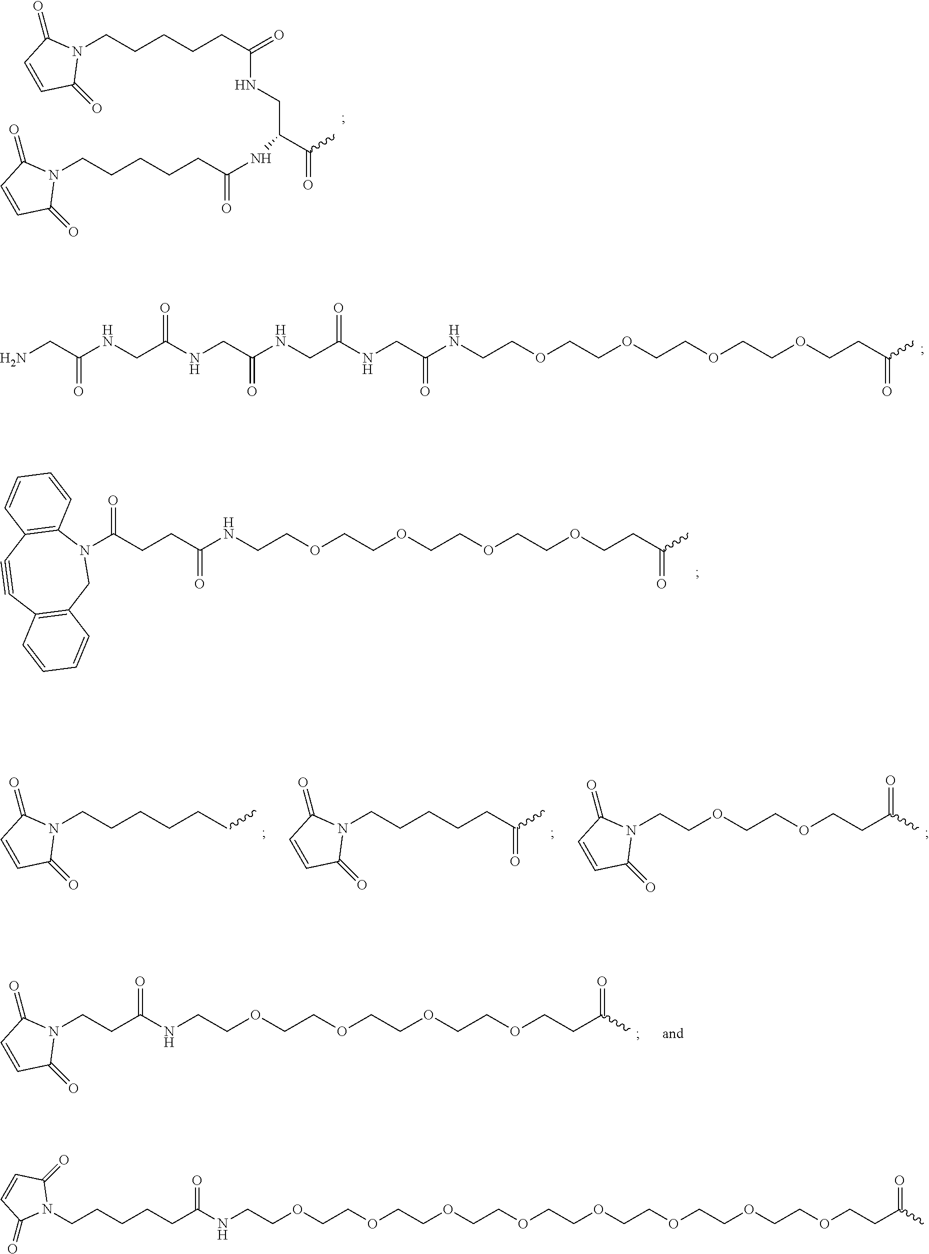

wherein the wavy line [symbol] indicates an attachment site of the Stretcher group to the enzyme-cleavable group, and the attachment site to the Targeting group is on the maleimide, primary amine or the alkyne functional group.

### Linker Compounds

In some embodiments, provided are Linker compounds selected from one of the following structures:

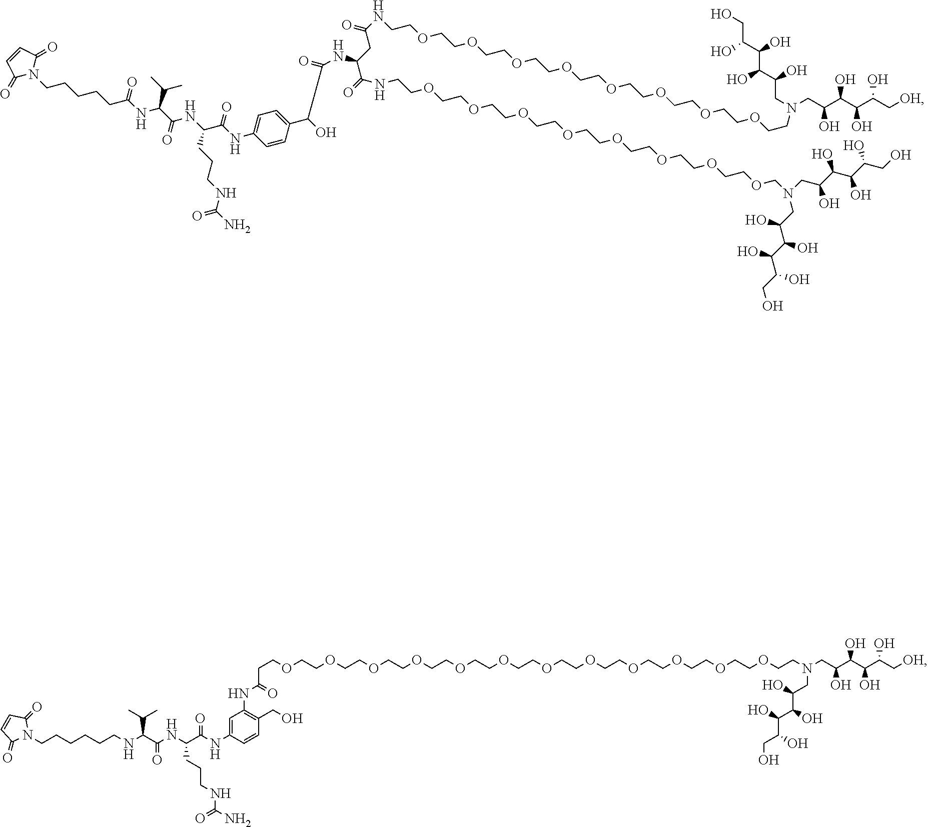

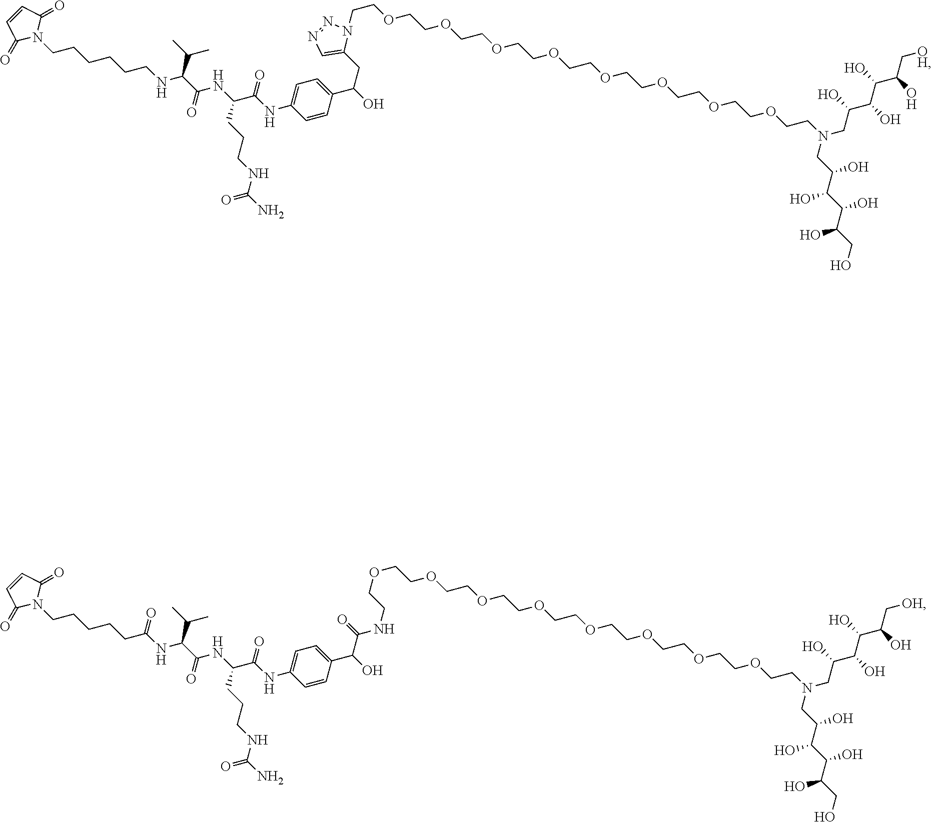 [SMILES: CC(C)[C@H](NCCCCCCN1C(=O)C=CC1=O)C(=O)N[C@@H](CCCNC(N)=O)C(=O)Nc1ccc(C(O)Cc2cnnn2CCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1 ; CC(C)[C@H](NC(=O)CCCCCN1C(=O)C=CC1=O)C(=O)N[C@@H](CCCNC(N)=O)C(=O)Nc1ccc(C(O)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

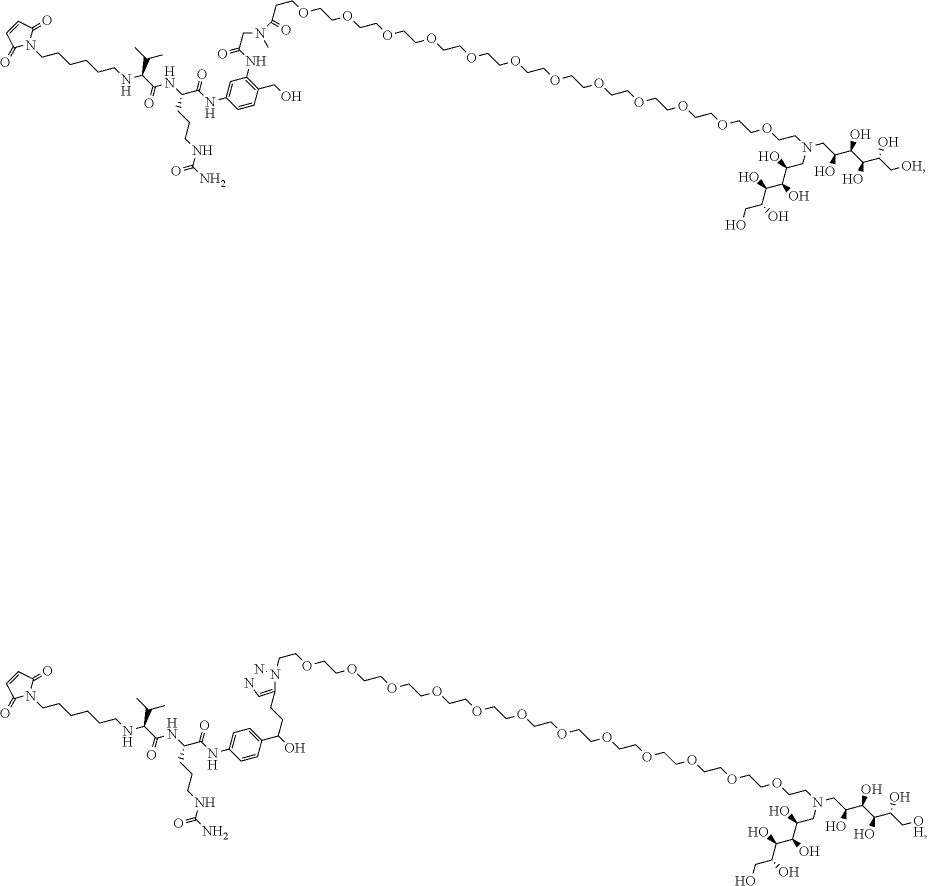 [SMILES: CC(C)[C@H](NCCCCCCN1C(=O)C=CC1=O)C(=O)N[C@@H](CCCNC(N)=O)C(=O)Nc1ccc(CO)c(NC(=O)CN(C)C(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1 ; CC(C)[C@H](NCCCCCCN1C(=O)C=CC1=O)C(=O)N[C@@H](CCCNC(N)=O)C(=O)Nc1ccc(C(O)CCc2cnnn2CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

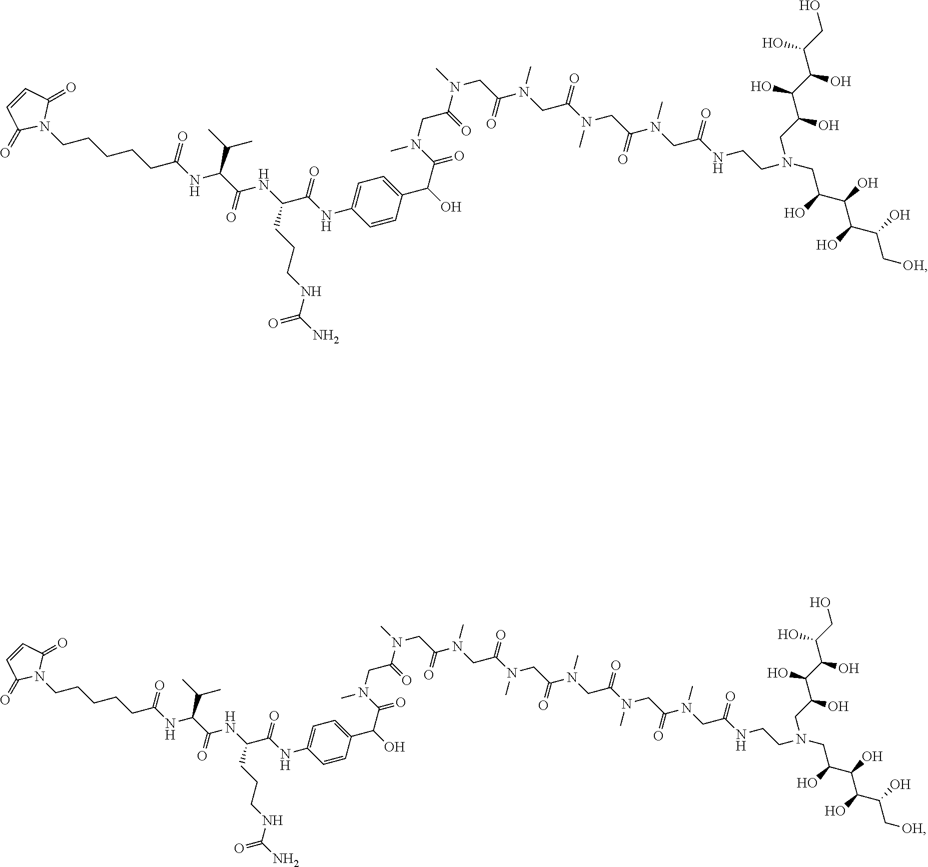 [SMILES: CC(C)[C@H](NC(=O)CCCCCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)cc1 ; CC(C)[C@H](NC(=O)CCCCCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)CC(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)cc1]

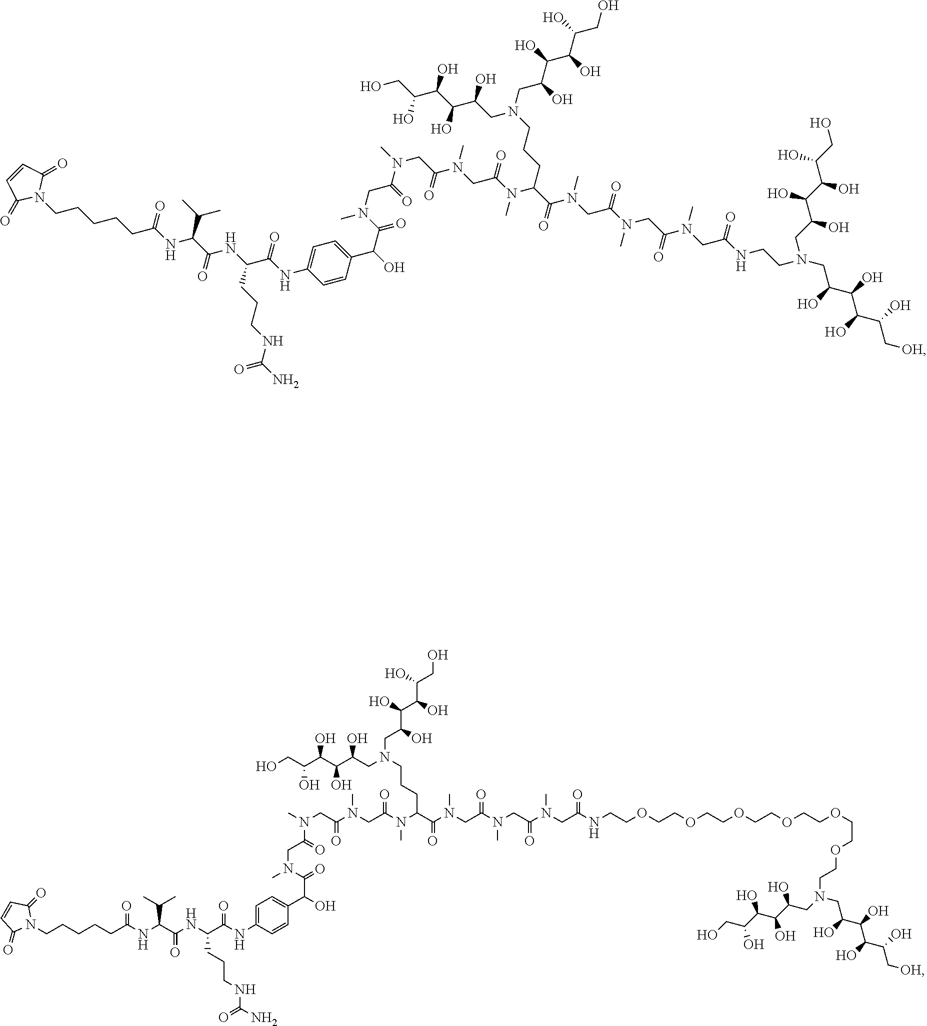

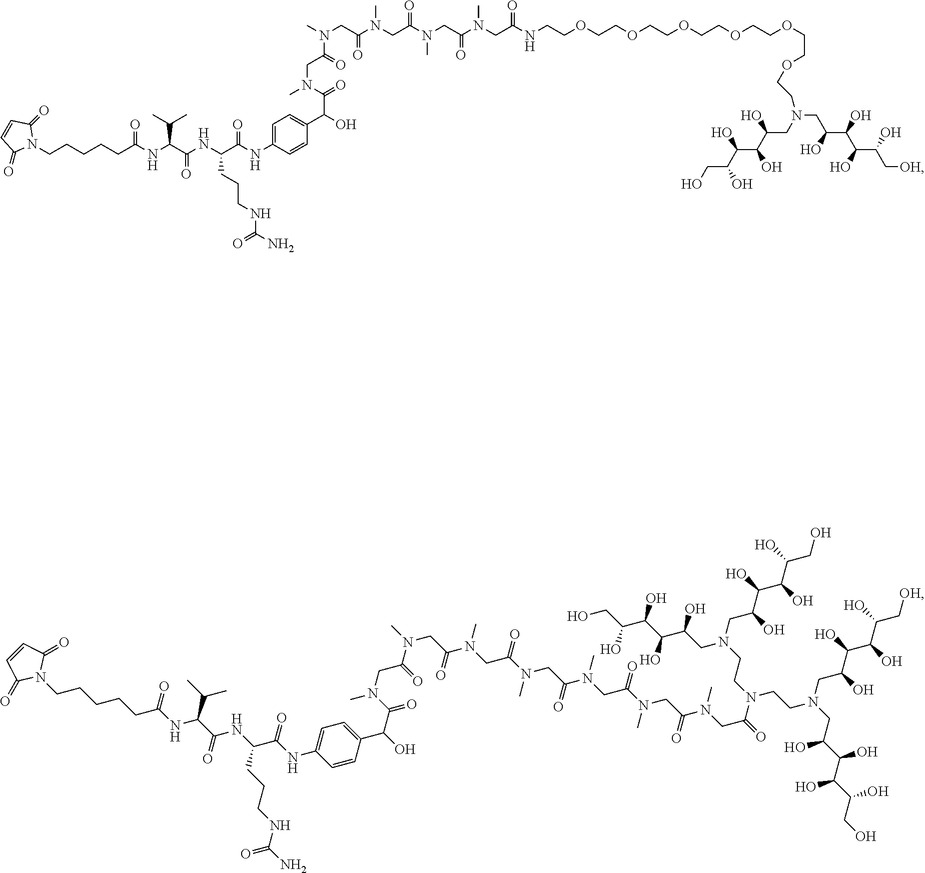

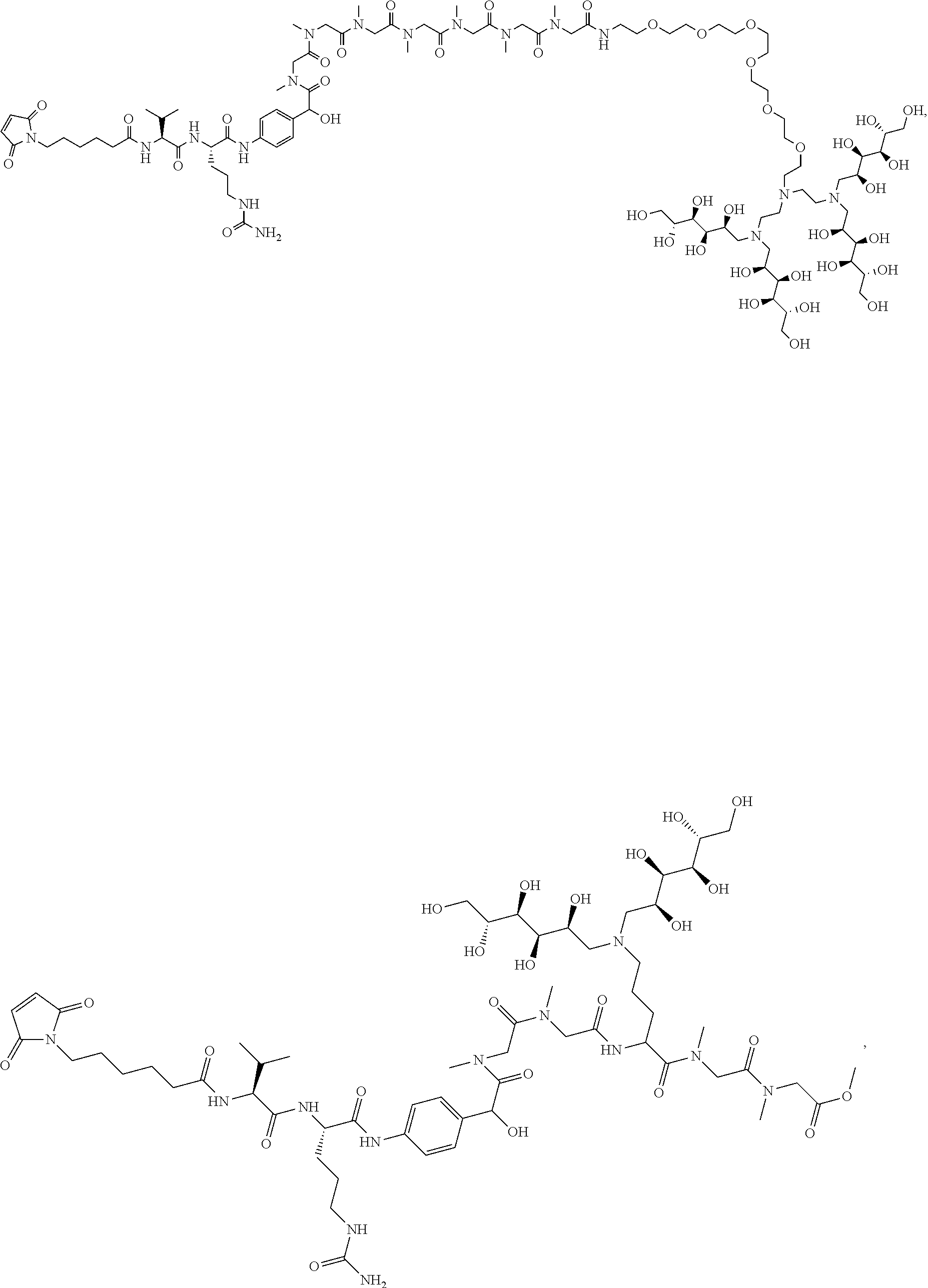

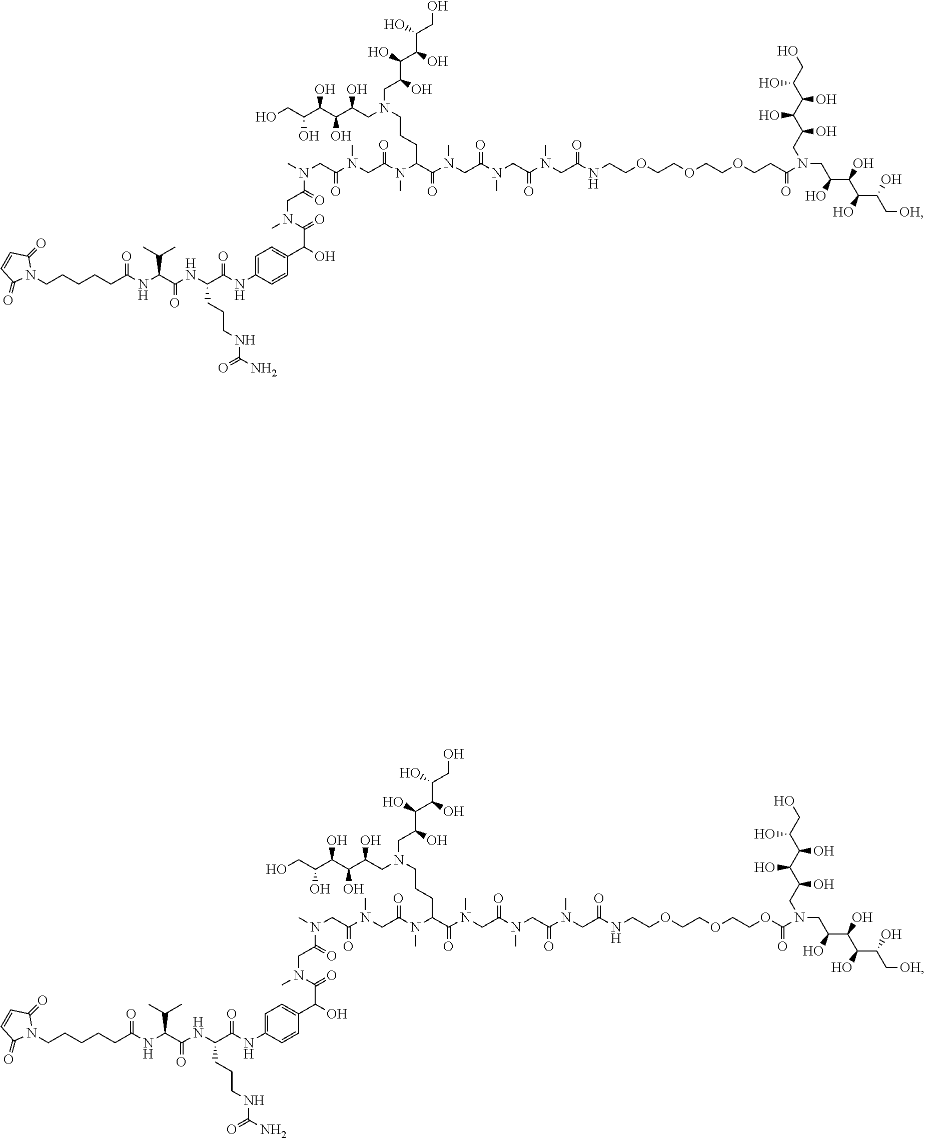

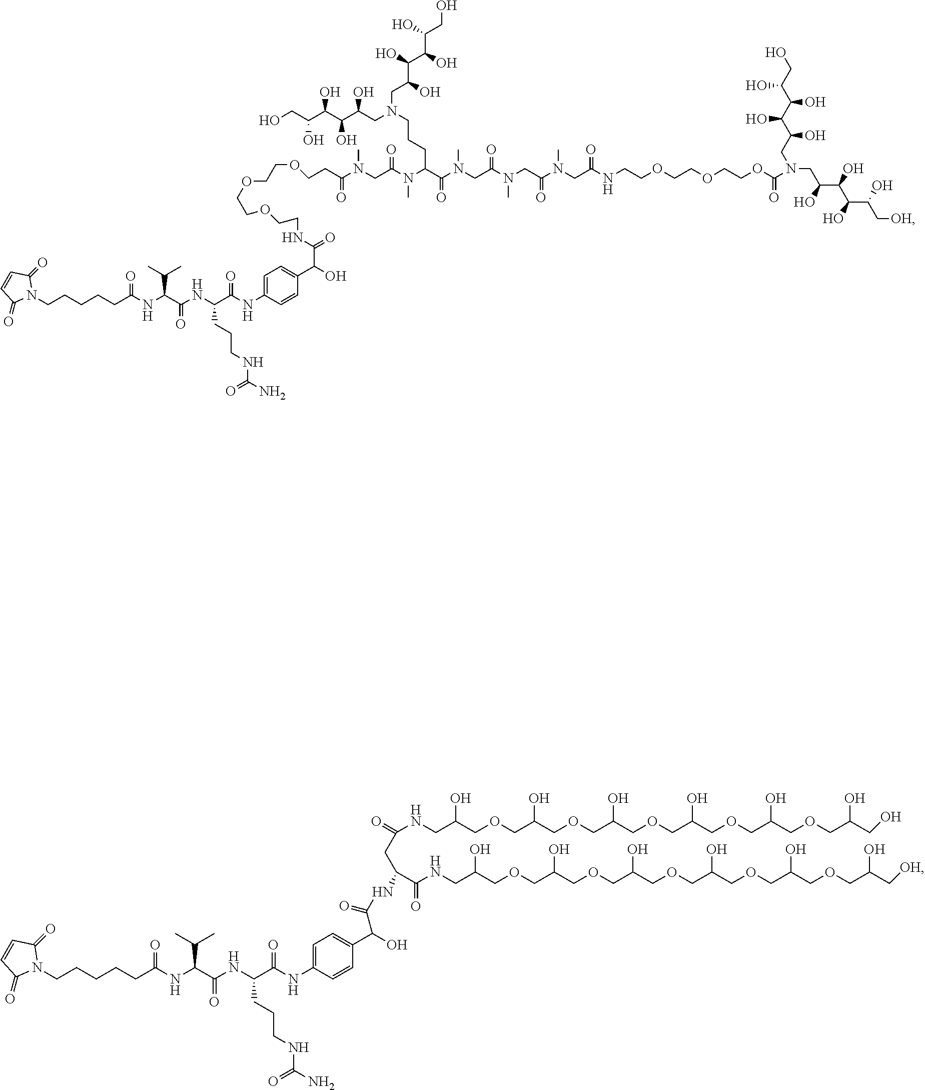

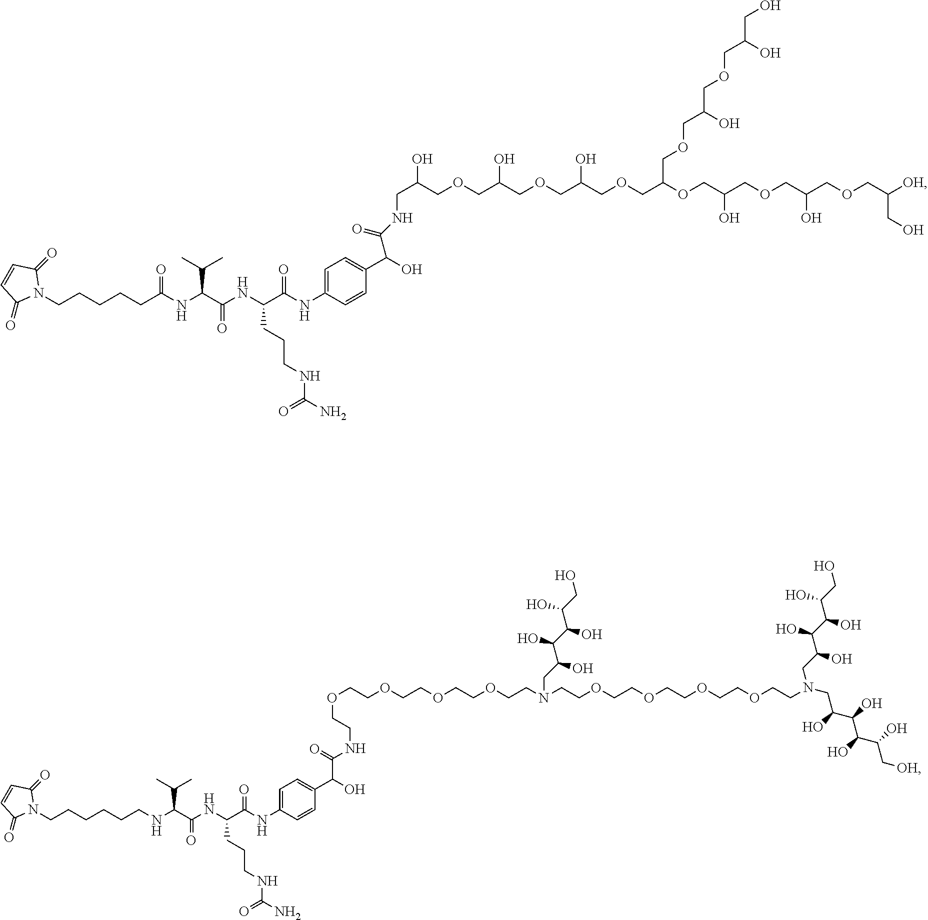 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCC(O)COCC(O)COCC(O)COCC(COCC(O)COCC(O)CO)OCC(O)COCC(O)COCC(O)CO)cc1 ; CC(C)[C@H](NCCCCCCN1C(=O)C=CC1=O)C(=O)N[C@@H](CCCNC(N)=O)C(=O)Nc1ccc(C(O)C(=O)NCCOCCOCCOCCOCCN(CCOCCOCCOCCOCCN(C[C@H](O)[C@@H](O)[C@H](O)[C@H](O)CO)C[C@H](O)[C@@H](O)[C@H](O)[C@H](O)CO)C[C@H](O)[C@@H](O)[C@H](O)[C@H](O)CO)cc1]

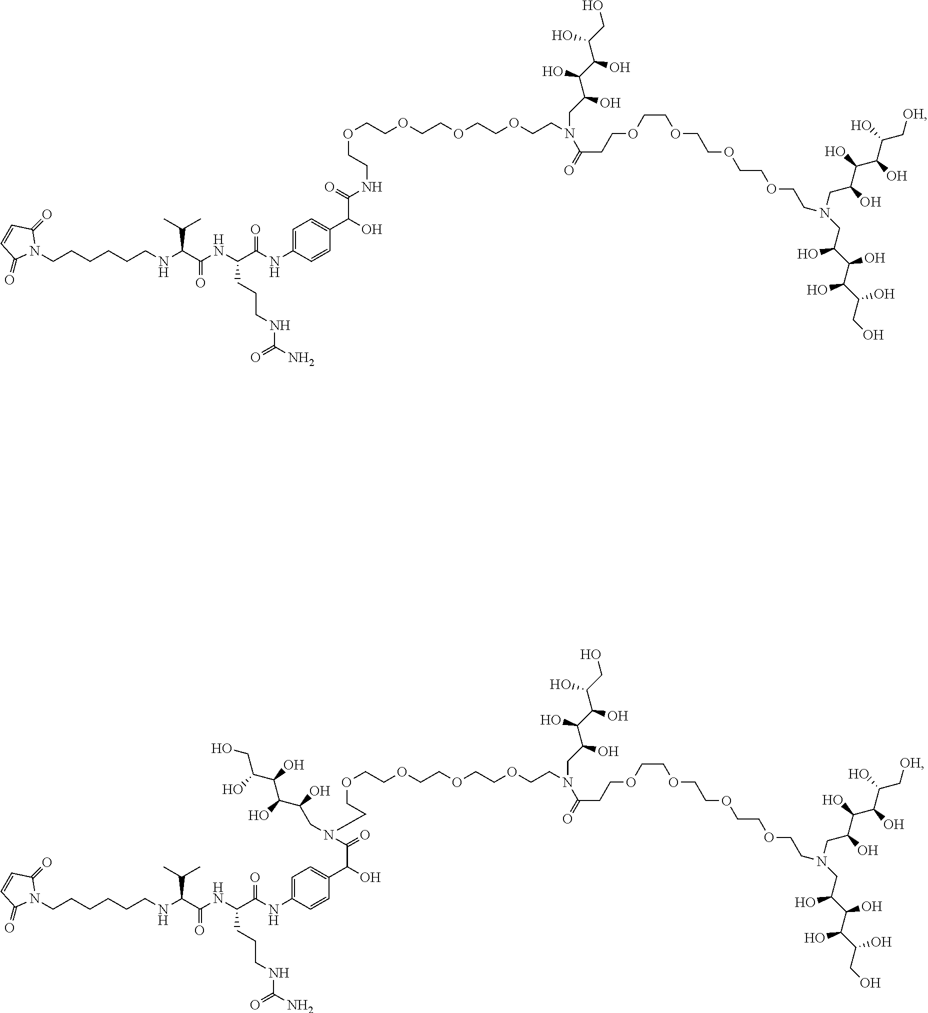

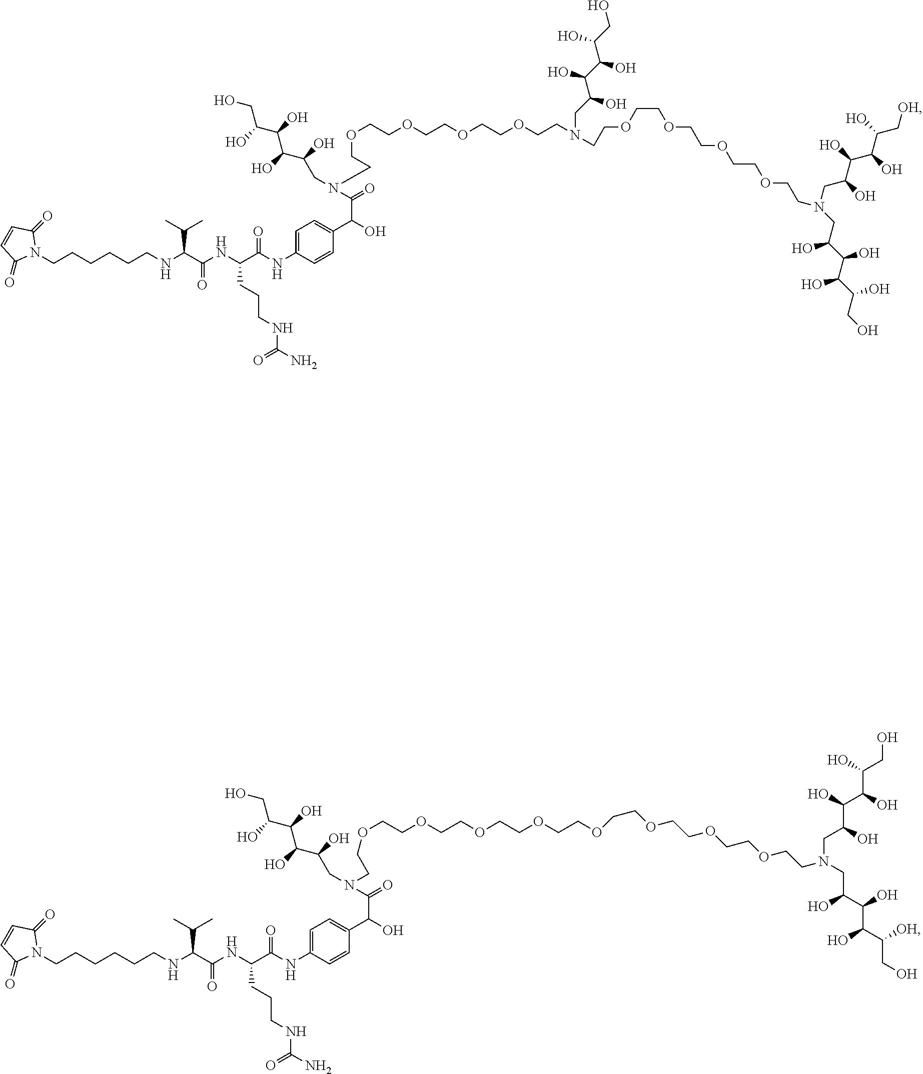 [SMILES: CC(C)[C@H](NCCCCCCN1C(=O)C=CC1=O)C(=O)N[C@@H](CCCNC(N)=O)C(=O)Nc1ccc(C(O)C(=O)N(CCOCCOCCOCCOCCN(CCOCCOCCOCCOCCN(C[C@H](O)[C@@H](O)[C@H](O)[C@H](O)CO)C[C@H](O)[C@@H](O)[C@H](O)[C@H](O)CO)C[C@H](O)[C@@H](O)[C@H](O)[C@H](O)CO)C[C@H](O)[C@@H](O)[C@H](O)[C@H](O)CO)cc1 ; CC(C)[C@H](NCCCCCCN1C(=O)C=CC1=O)C(=O)N[C@@H](CCCNC(N)=O)C(=O)Nc1ccc(C(O)C(=O)N(CCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C[C@H](O)[C@@H](O)[C@H](O)[C@H](O)CO)cc1]

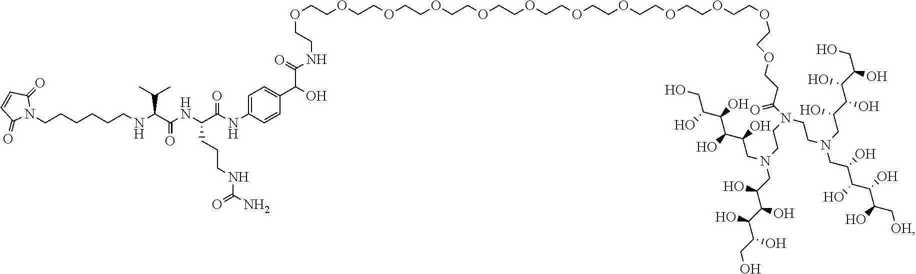 [SMILES: CC(C)[C@H](NCCCCCCN1C(=O)C=CC1=O)C(=O)N[C@@H](CCCNC(N)=O)C(=O)Nc1ccc(C(O)C(=O)NCCOCCOCCOCCOCCOCCOCCOCCOCCOCCOCCOCCOCCC(=O)N(CCN(C[C@H](O)[C@@H](O)[C@H](O)[C@H](O)CO)C[C@H](O)[C@@H](O)[C@H](O)[C@H](O)CO)CCN(C[C@H](O)[C@@H](O)[C@H](O)[C@H](O)CO)C[C@H](O)[C@@H](O)[C@H](O)[C@H](O)CO)cc1]

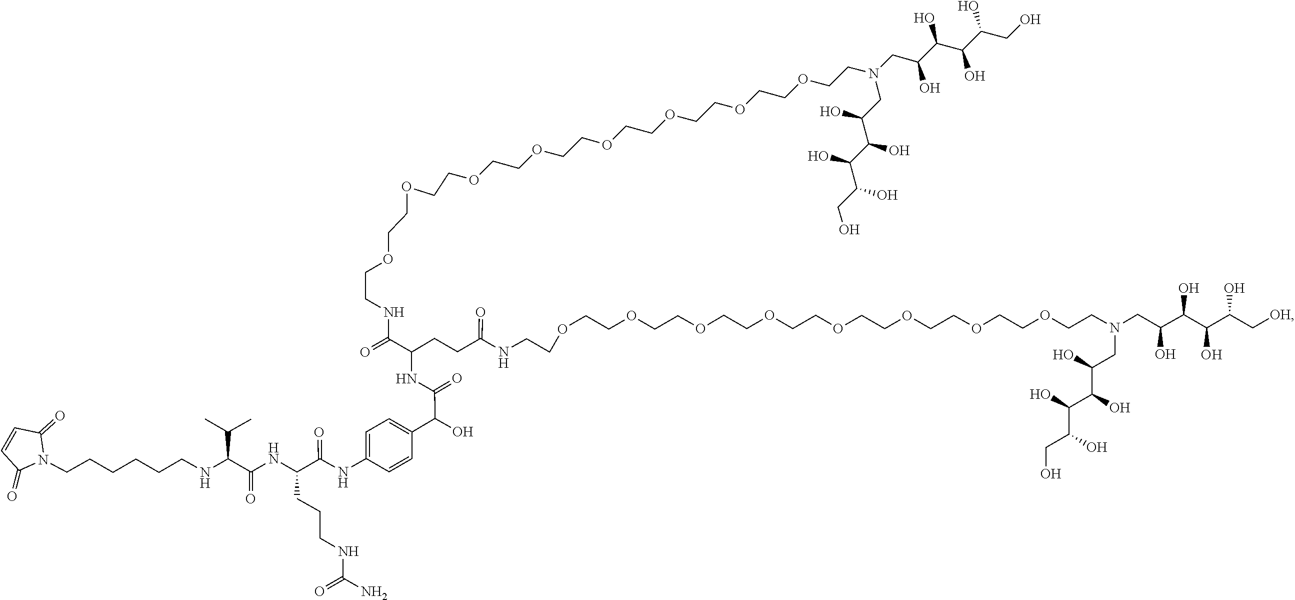 [SMILES: CC(C)[C@H](NCCCCCCN1C(=O)C=CC1=O)C(=O)N[C@@H](CCCNC(N)=O)C(=O)Nc1ccc(C(O)C(=O)NC(CCC(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

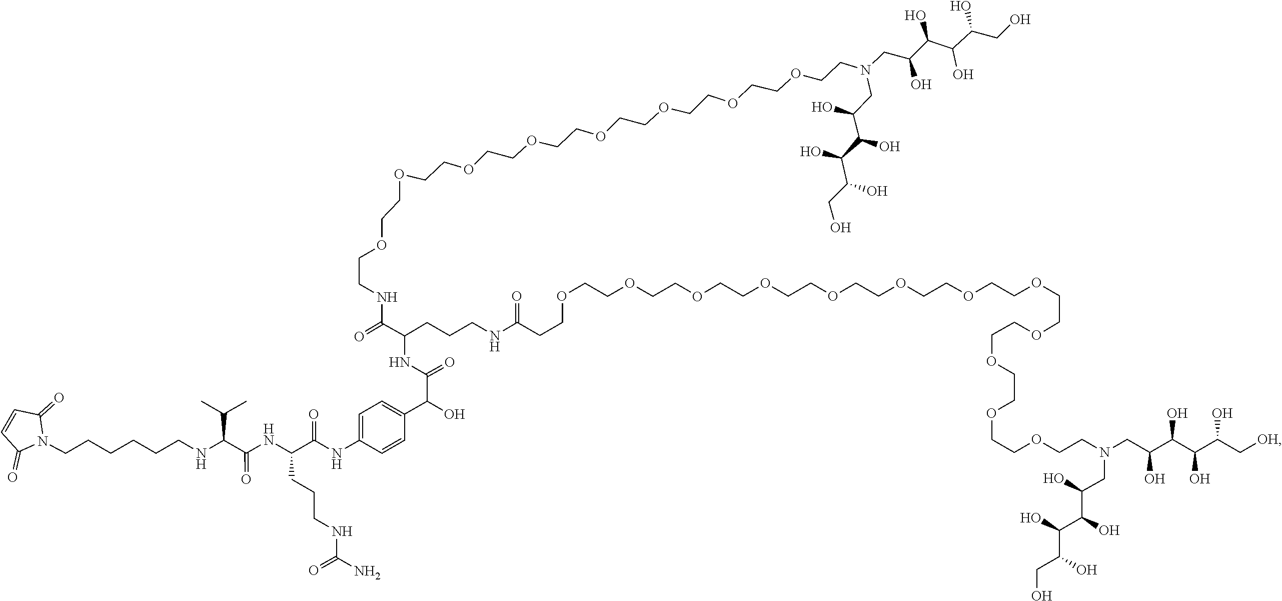 [SMILES: CC(C)[C@H](NCCCCCCN1C(=O)C=CC1=O)C(=O)N[C@@H](CCCNC(N)=O)C(=O)Nc1ccc(C(O)C(=O)NC(CCCNC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)C(O)[C@H](O)CO)cc1]

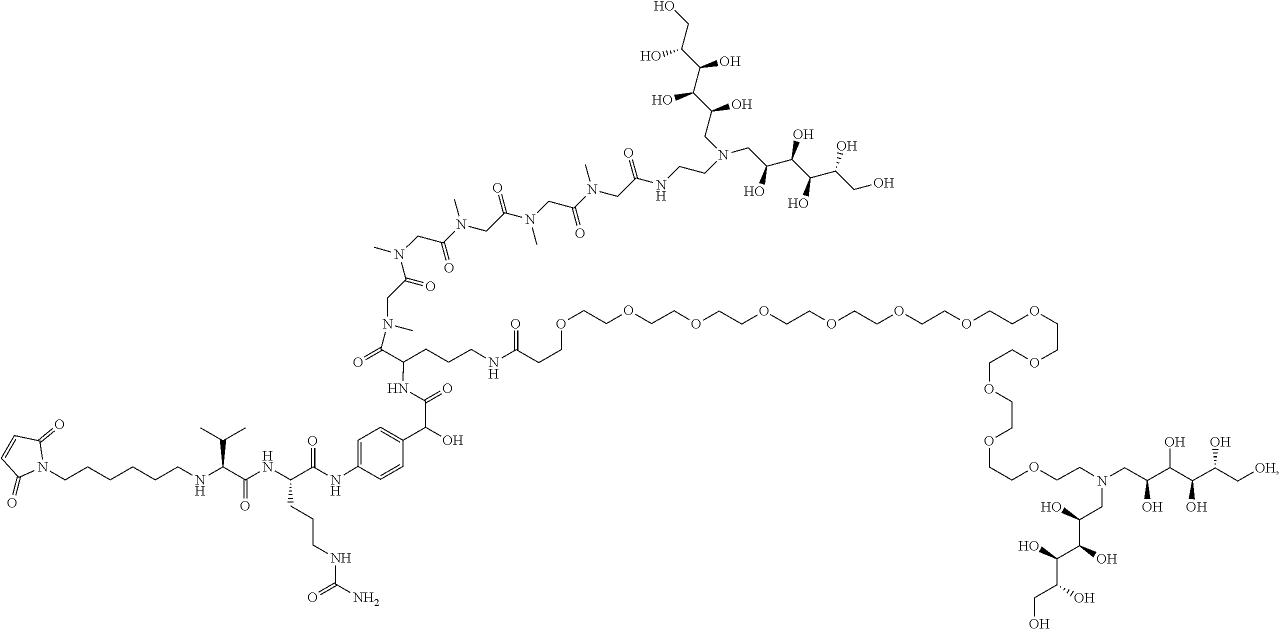 [SMILES: CC(C)[C@H](NCCCCCCN1C(=O)C=CC1=O)C(=O)N[C@@H](CCCNC(N)=O)C(=O)Nc1ccc(C(O)C(=O)NC(CCCNC(=O)CCOCCOCCOCCOCCOCCOCCOCCOCCOCCOCCOCCOCCN(C[C@H](O)C(O)[C@H](O)[C@H](O)CO)C[C@H](O)[C@@H](O)[C@H](O)[C@H](O)CO)C(=O)N(C)CC(=O)N(C)CC(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)cc1]

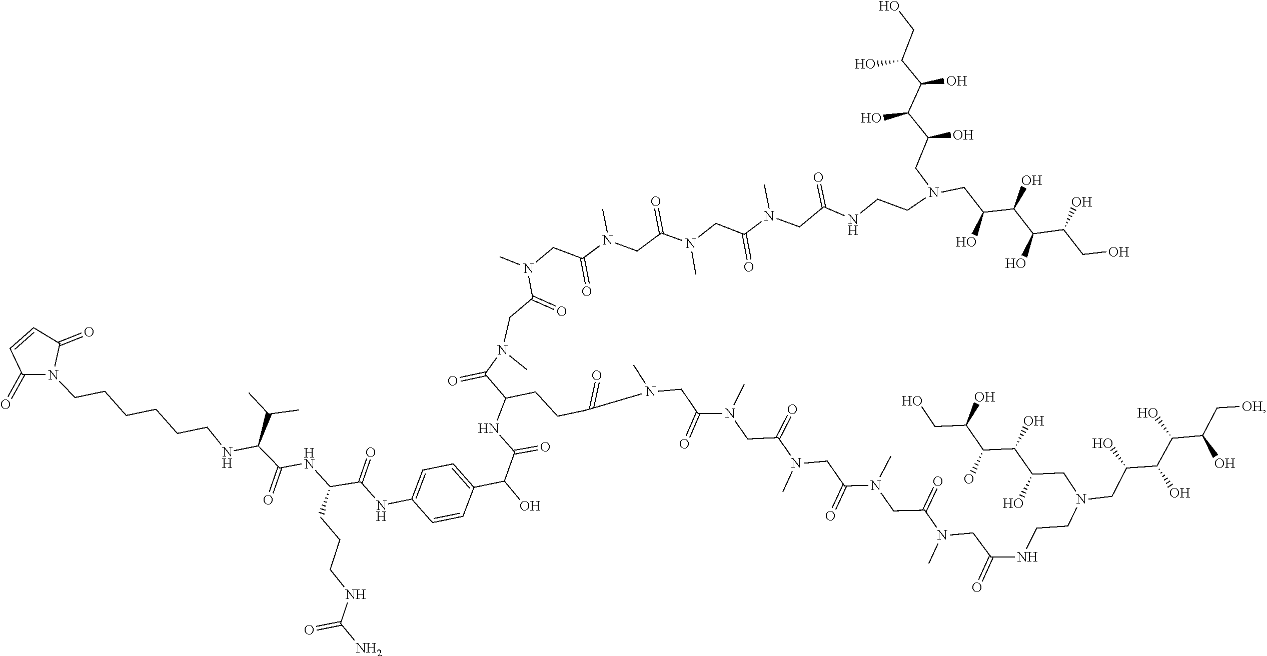

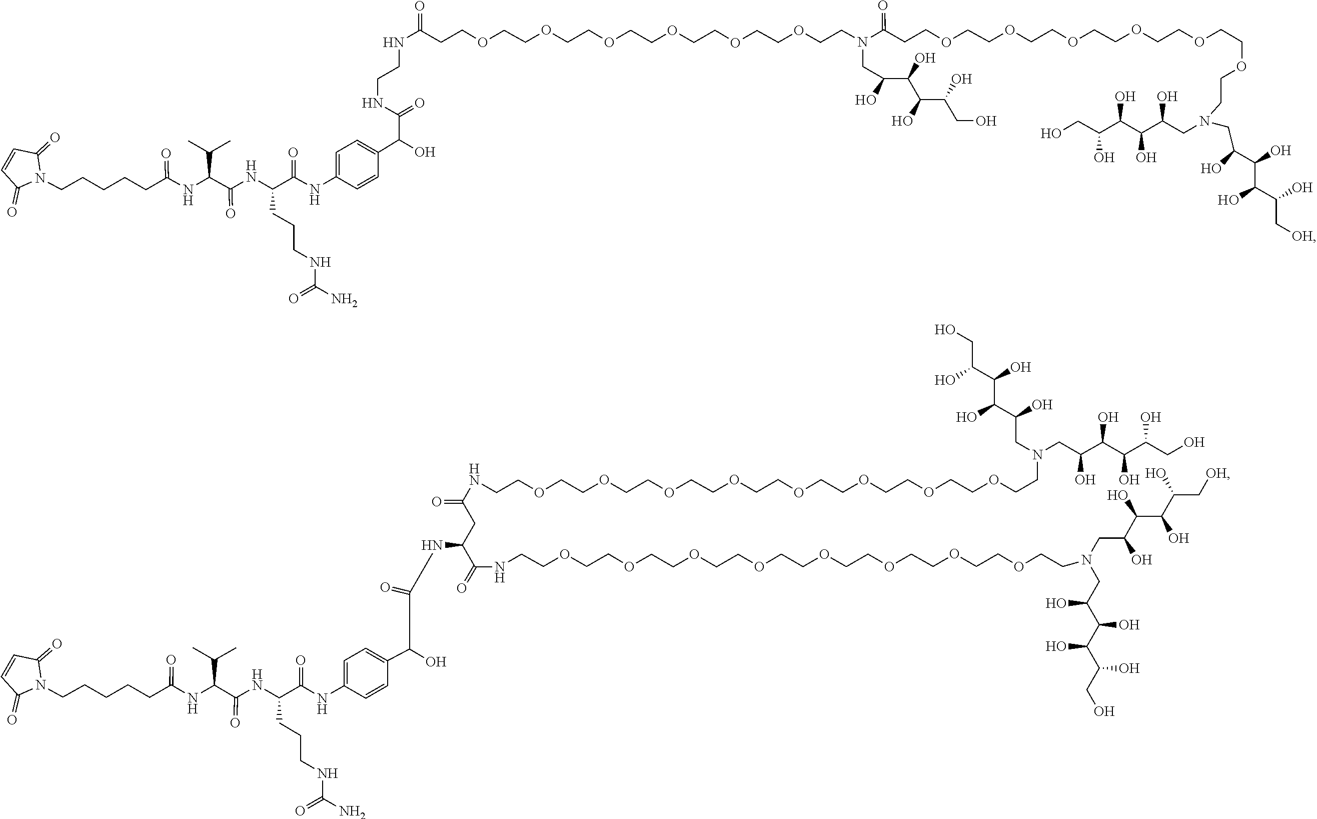

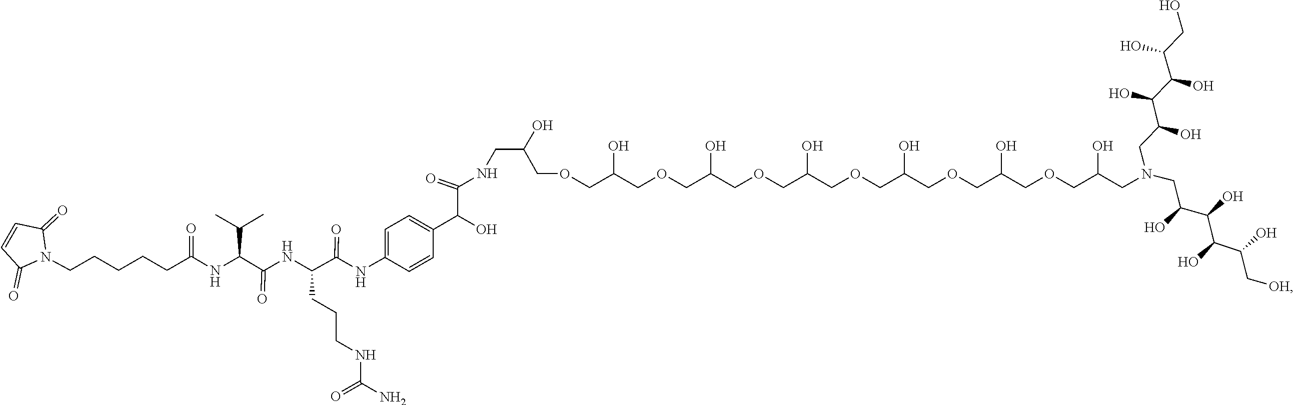 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCC(O)COCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)cc1]

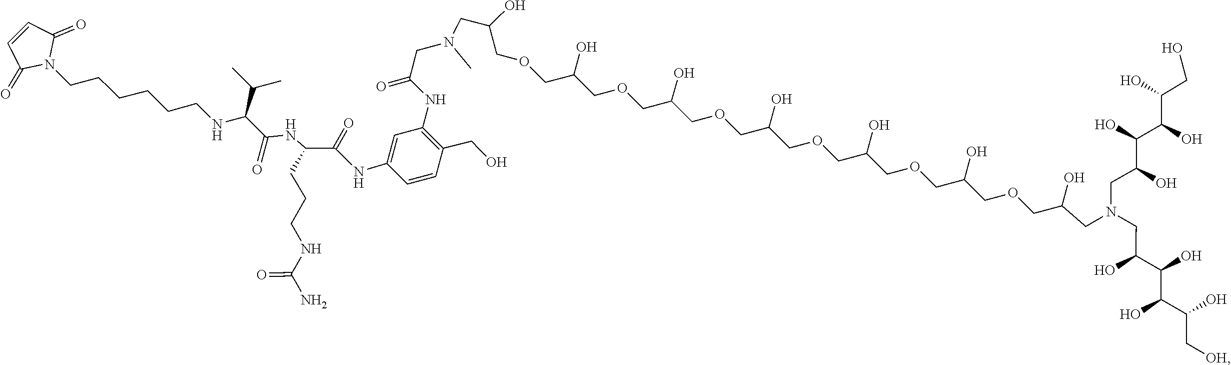 [SMILES: CC(C)[C@H](NCCCCCCN1C(=O)C=CC1=O)C(=O)N[C@@H](CCCNC(N)=O)C(=O)Nc1ccc(CO)c(NC(=O)CN(C)CC(O)COCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)c1]

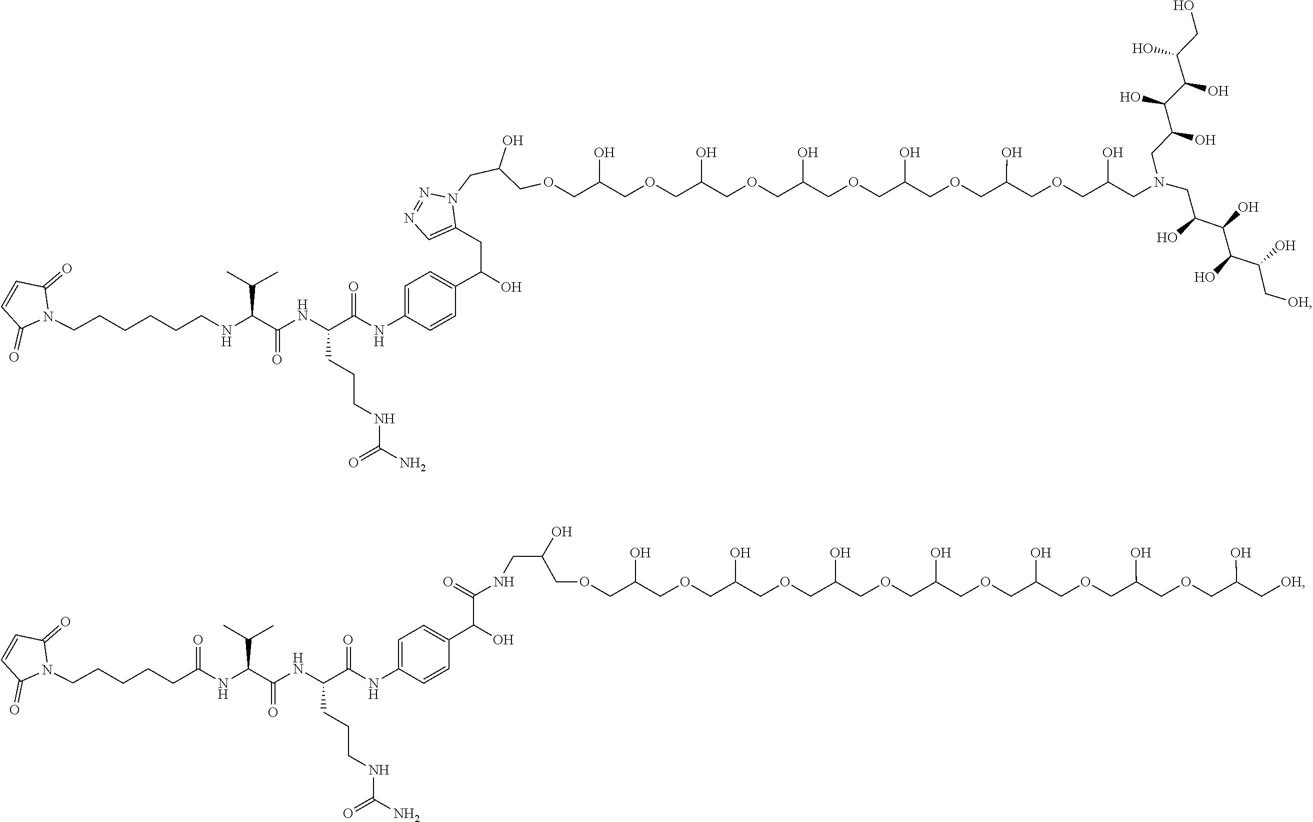 [SMILES: CC(C)[C@H](NCCCCCCN1C(=O)C=CC1=O)C(=O)N[C@@H](CCCNC(N)=O)C(=O)Nc1ccc(C(O)Cc2cnnn2CC(O)COCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)cc1 ; CC(C)[C@H](NC(=O)CCCCCN1C(=O)C=CC1=O)C(=O)N[C@@H](CCCNC(N)=O)C(=O)Nc1ccc(C(O)C(=O)NCC(O)COCC(O)COCC(O)COCC(O)COCC(O)COCC(O)COCC(O)COCC(O)CO)cc1]

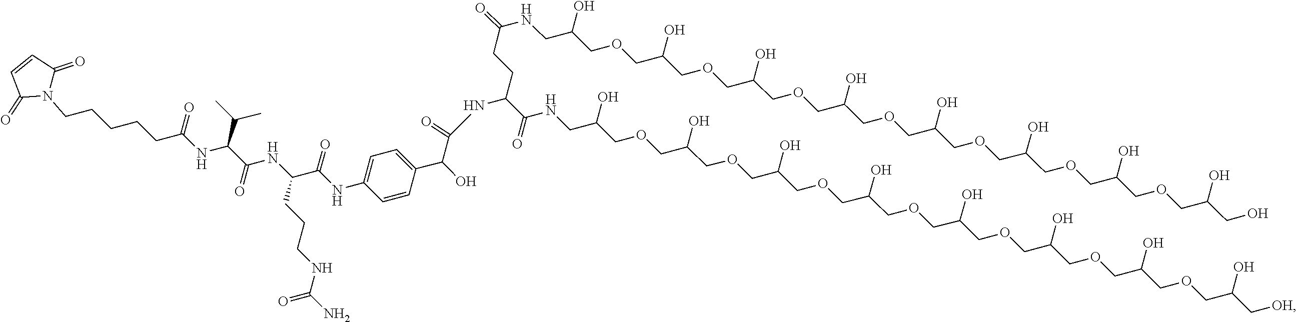 [SMILES: CC(C)[C@H](NC(=O)CCCCCN1C(=O)C=CC1=O)C(=O)N[C@@H](CCCNC(N)=O)C(=O)Nc1ccc(C(O)C(=O)NC(CCC(=O)NCC(O)COCC(O)COCC(O)COCC(O)COCC(O)COCC(O)COCC(O)COCC(O)CO)C(=O)NCC(O)COCC(O)COCC(O)COCC(O)COCC(O)COCC(O)COCC(O)COCC(O)CO)cc1]

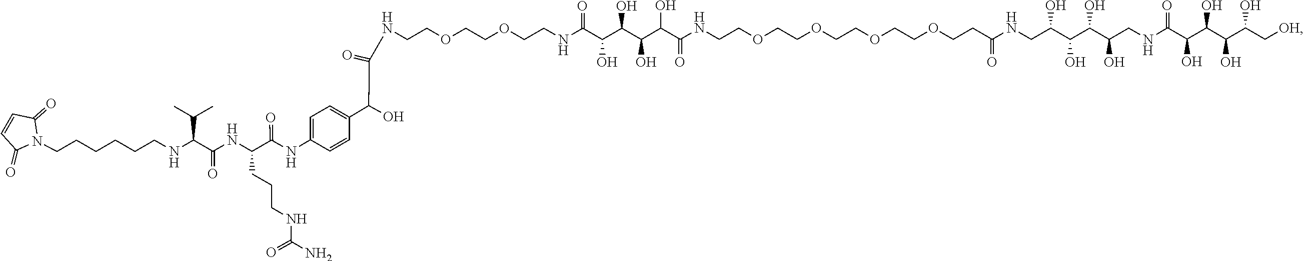 [SMILES: CC(C)[C@H](NCCCCCCN1C(=O)C=CC1=O)C(=O)N[C@@H](CCCNC(N)=O)C(=O)Nc1ccc(C(O)C(=O)NCCOCCOCCNC(=O)[C@@H](O)[C@@H](O)[C@H](O)C(O)C(=O)NCCOCCOCCOCCOCCC(=O)NC[C@H](O)[C@@H](O)[C@H](O)[C@H](O)CNC(=O)[C@H](O)[C@@H](O)[C@H](O)[C@H](O)CO)cc1]

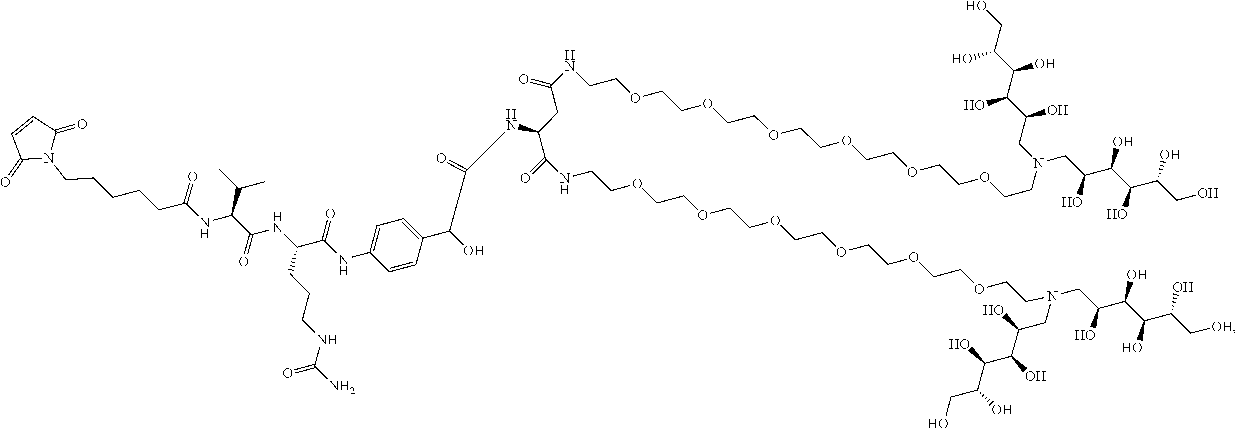 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@@H](CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

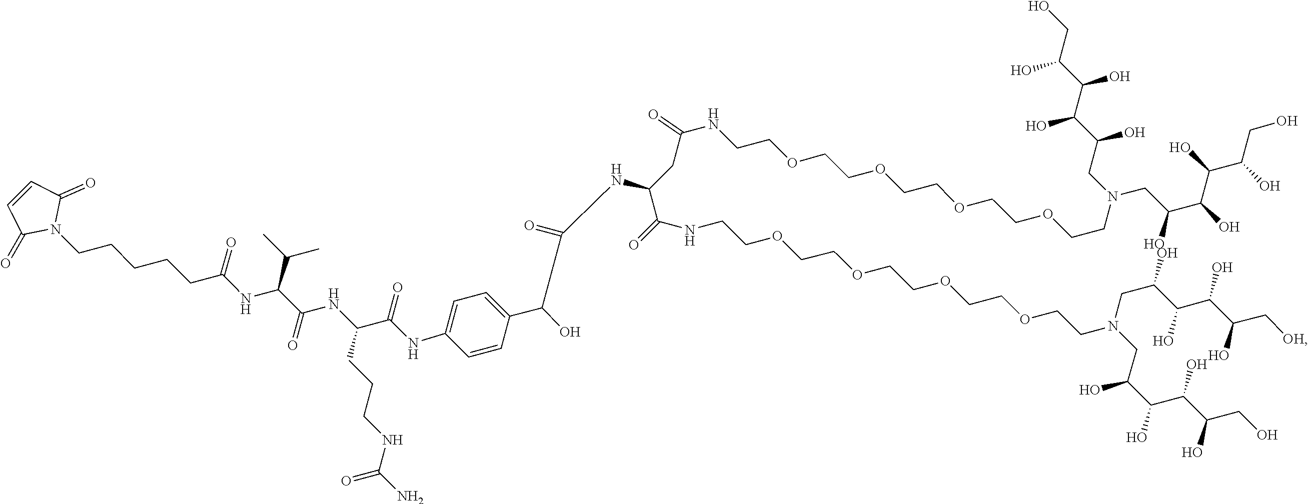 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@@H](CC(=O)NCCOCCOCCOCCOCCN(C[C@H](O)[C@H](O)[C@@H](O)[C@@H](O)CO)C[C@H](O)[C@@H](O)[C@H](O)[C@H](O)CO)C(=O)NCCOCCOCCOCCOCCN(C[C@H](O)[C@@H](O)[C@H](O)[C@H](O)CO)C[C@H](O)[C@@H](O)[C@H](O)[C@H](O)CO)cc1]

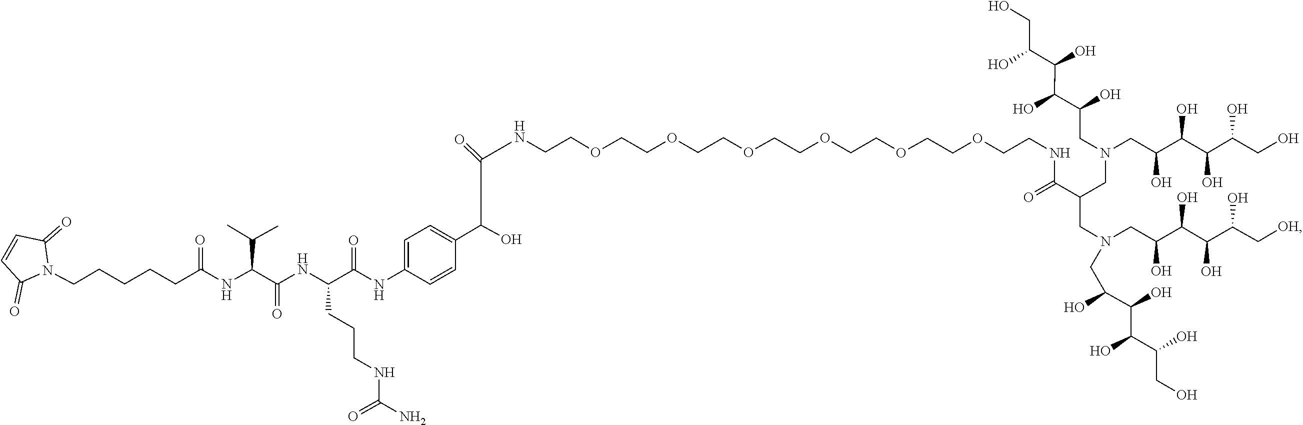 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCCOCCOCCOCCOCCOCCOCCNC(=O)C(CN(C[C@H](O)[C@@H](O)[C@H](O)[C@H](O)CO)C[C@H](O)[C@@H](O)[C@H](O)[C@H](O)CO)CN(C[C@H](O)[C@@H](O)[C@H](O)[C@H](O)CO)C[C@H](O)[C@@H](O)[C@H](O)[C@H](O)CO)cc1]

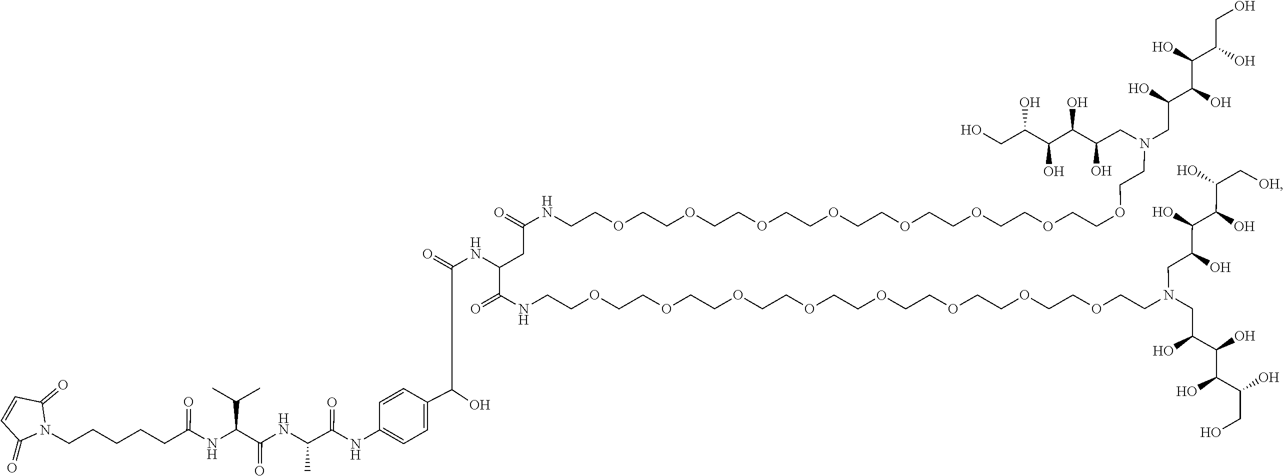 [SMILES: CC(C)[C@H](NC(=O)CCCCCN1C(=O)C=CC1=O)C(=O)N[C@@H](C)C(=O)Nc1ccc(C(O)C(=O)NC(CC(=O)NCCOCCOCCOCCOCCOCCOCCOCCOCCN(C[C@@H](O)[C@H](O)[C@@H](O)[C@@H](O)CO)C[C@@H](O)[C@H](O)[C@@H](O)[C@@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

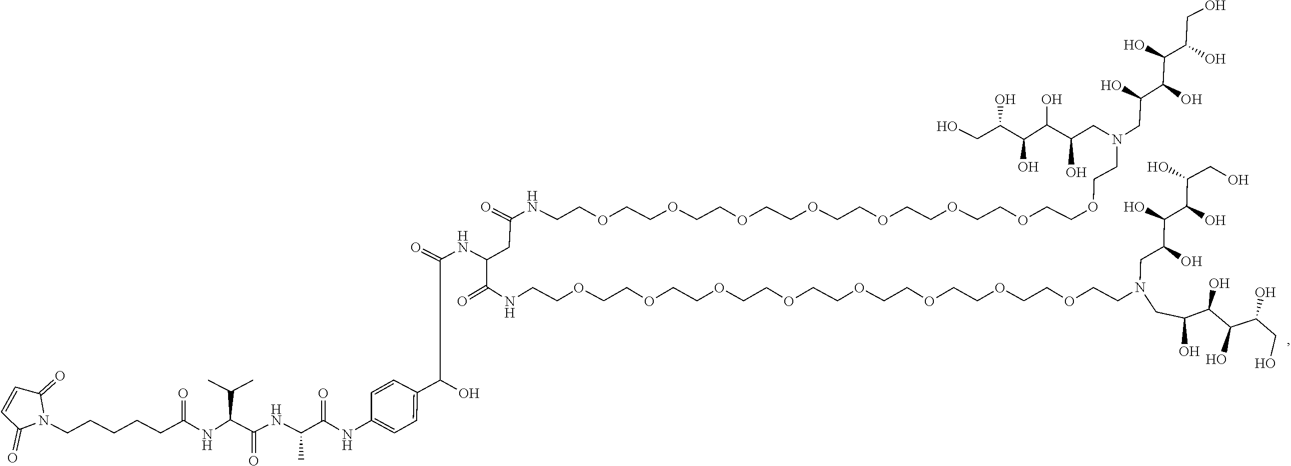 [SMILES: CC(C)[C@H](NC(=O)CCCCCN1C(=O)C=CC1=O)C(=O)N[C@@H](C)C(=O)Nc1ccc(C(O)C(=O)NC(CC(=O)NCCOCCOCCOCCOCCOCCOCCOCCOCCN(C[C@@H](O)C(O)[C@@H](O)[C@@H](O)CO)C[C@@H](O)[C@H](O)[C@@H](O)[C@@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

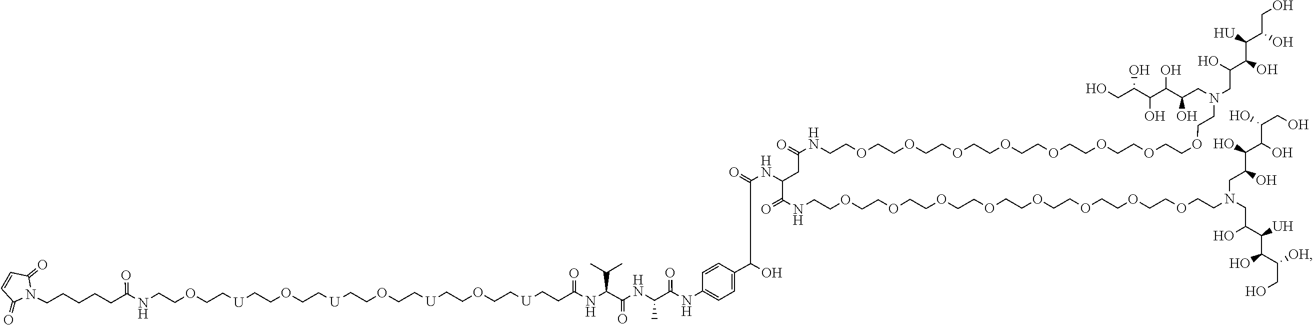

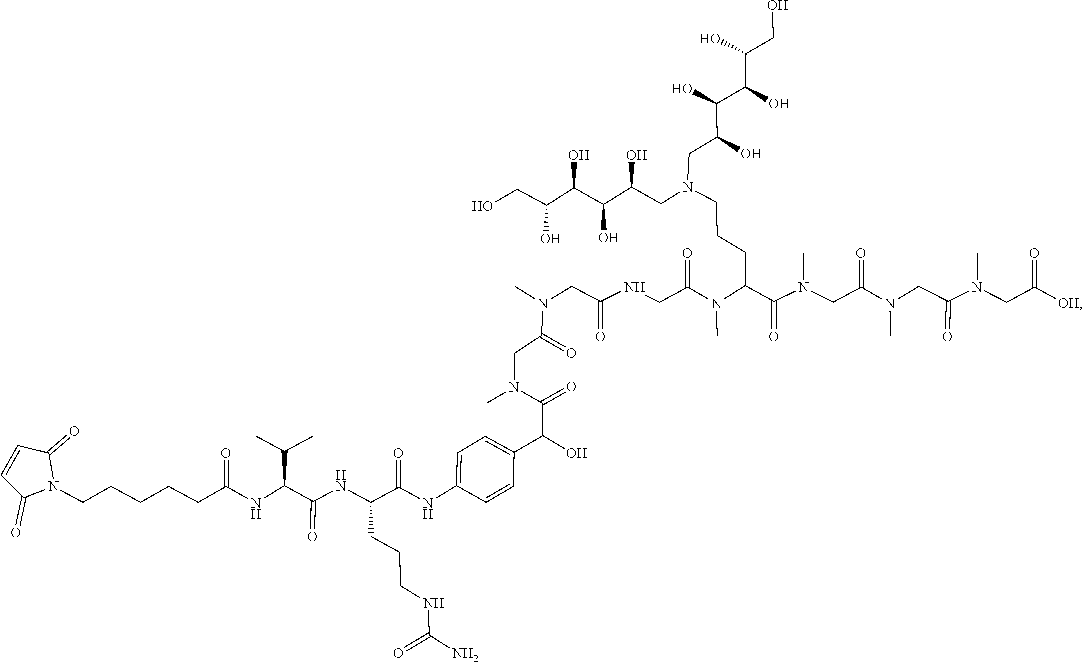 [SMILES: CC(C)[C@H](NC(=O)CCCCCN1C(=O)C=CC1=O)C(=O)N[C@@H](CCCNC(N)=O)C(=O)Nc1ccc(C(O)C(=O)N(C)CC(=O)N(C)CC(=O)NCC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)O)cc1]

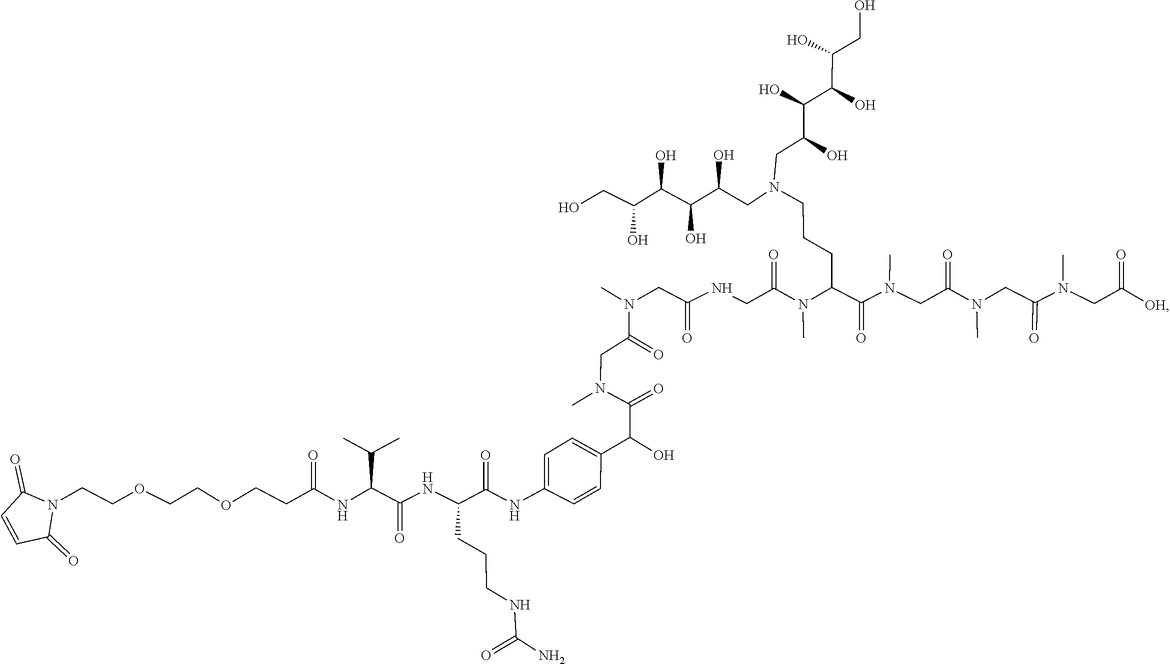 [SMILES: CC(C)[C@H](NC(=O)CCOCCOCCN1C(=O)C=CC1=O)C(=O)N[C@@H](CCCNC(N)=O)C(=O)Nc1ccc(C(O)C(=O)N(C)CC(=O)N(C)CC(=O)NCC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)O)cc1]

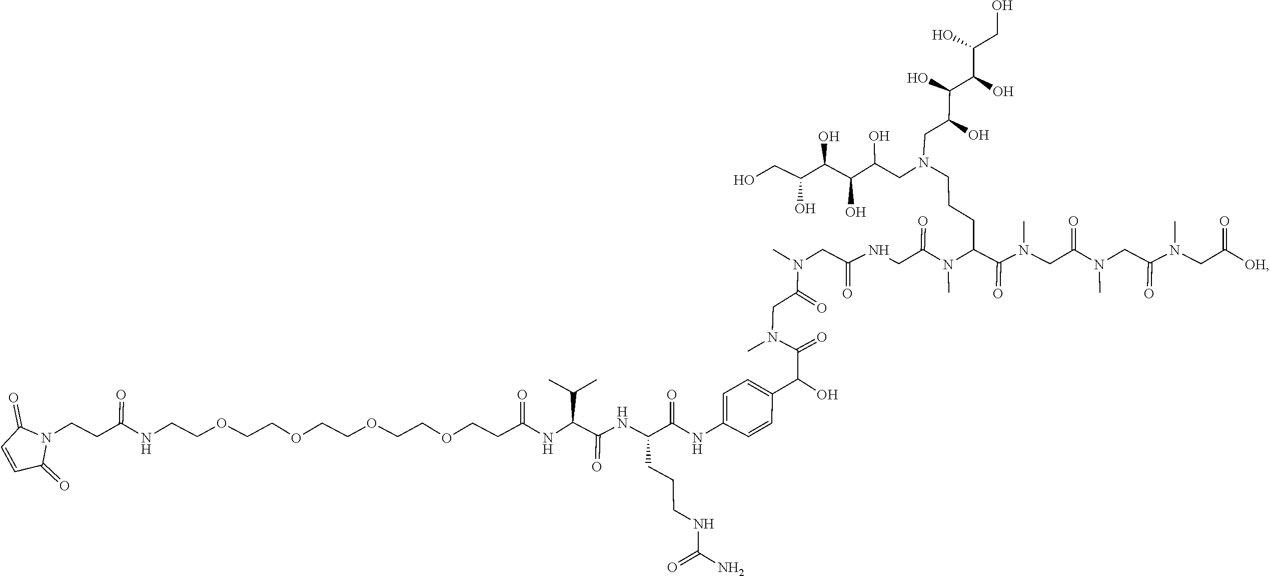 [SMILES: CC(C)[C@H](NC(=O)CCOCCOCCOCCOCCNC(=O)CCN1C(=O)C=CC1=O)C(=O)N[C@@H](CCCNC(N)=O)C(=O)Nc1ccc(C(O)C(=O)N(C)CC(=O)N(C)CC(=O)NCC(=O)N(C)C(CCCN(CC(O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)O)cc1]

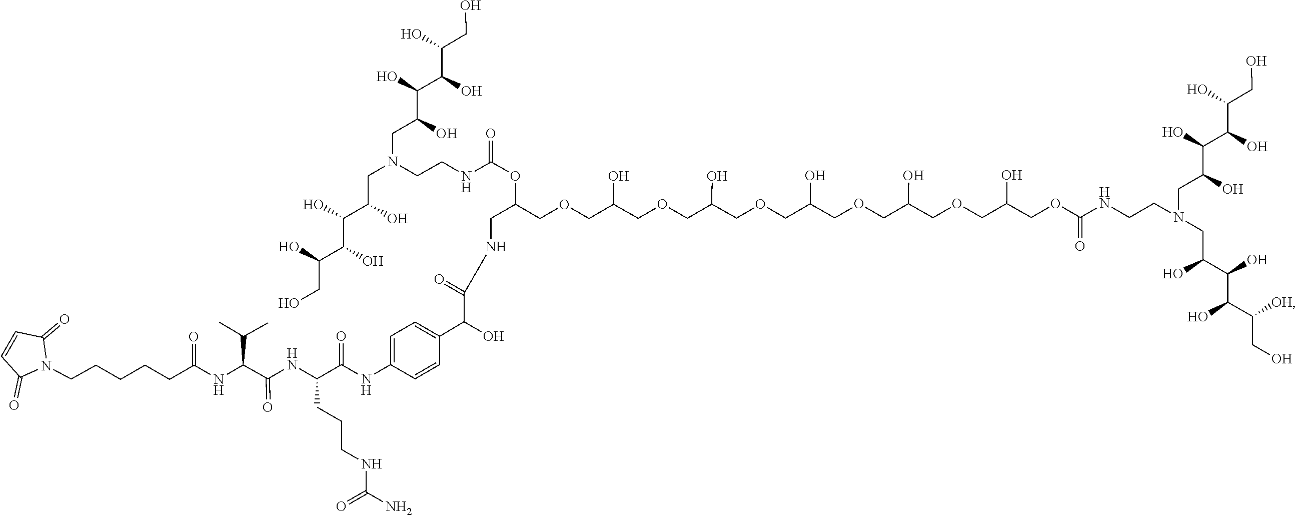 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCC(COCC(O)COCC(O)COCC(O)COCC(O)COCC(O)COC(=O)NCCN(C[C@H](O)[C@@H](O)[C@H](O)[C@H](O)CO)C[C@H](O)[C@@H](O)[C@H](O)[C@H](O)CO)OC(=O)NCCN(C[C@H](O)[C@@H](O)[C@H](O)[C@H](O)CO)C[C@H](O)[C@@H](O)[C@H](O)[C@H](O)CO)cc1]

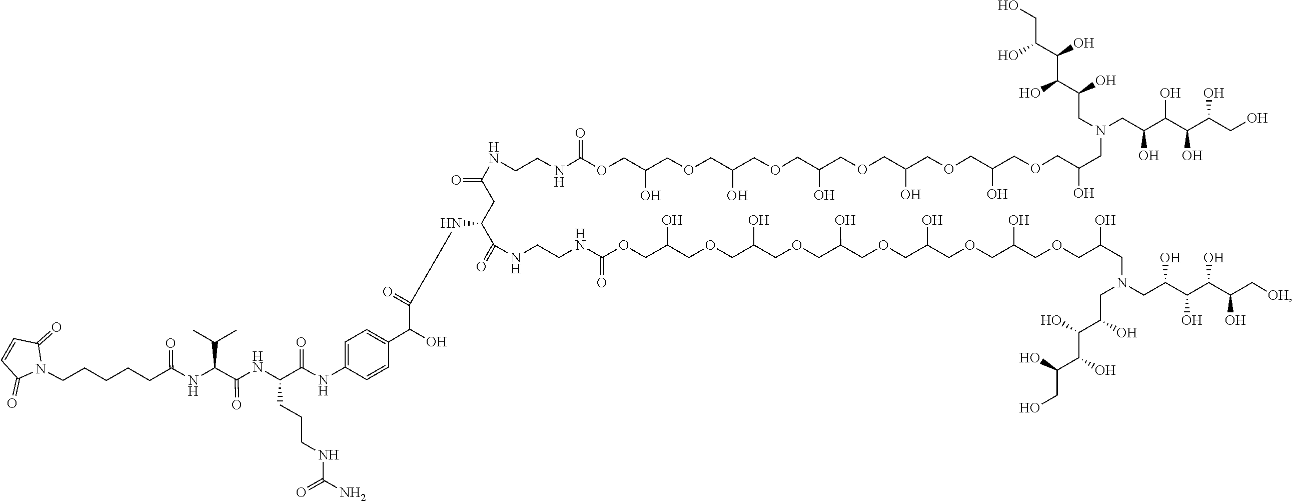 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCCNC(=O)OCC(O)COCC(O)COCC(O)COCC(O)COCC(O)COCC(O)CN(C[C@H](O)C(O)[C@H](O)[C@H](O)CO)C[C@H](O)[C@@H](O)[C@H](O)[C@H](O)CO)C(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)cc1]

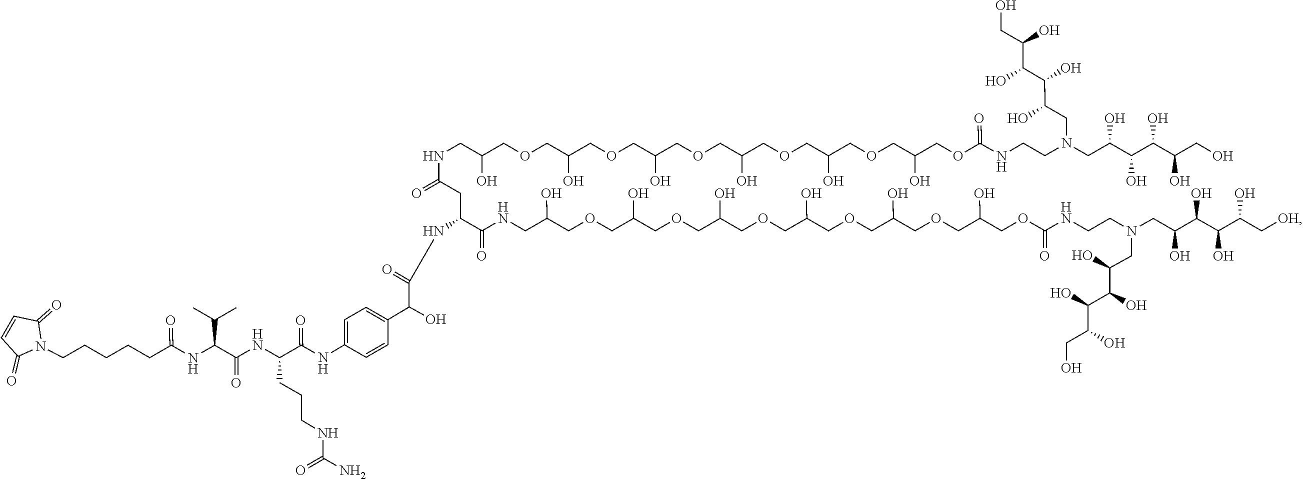 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCC(O)COCC(O)COCC(O)COCC(O)COCC(O)COCC(O)COC(=O)NCCN(C[C@H](O)[C@@H](O)[C@H](O)[C@H](O)CO)C[C@H](O)[C@@H](O)[C@H](O)[C@H](O)CO)C(=O)NCC(O)COCC(O)COCC(O)COCC(O)COCC(O)COCC(O)COC(=O)NCCN(C[C@H](O)[C@@H](O)[C@H](O)[C@H](O)CO)C[C@H](O)[C@@H](O)[C@H](O)[C@H](O)CO)cc1]

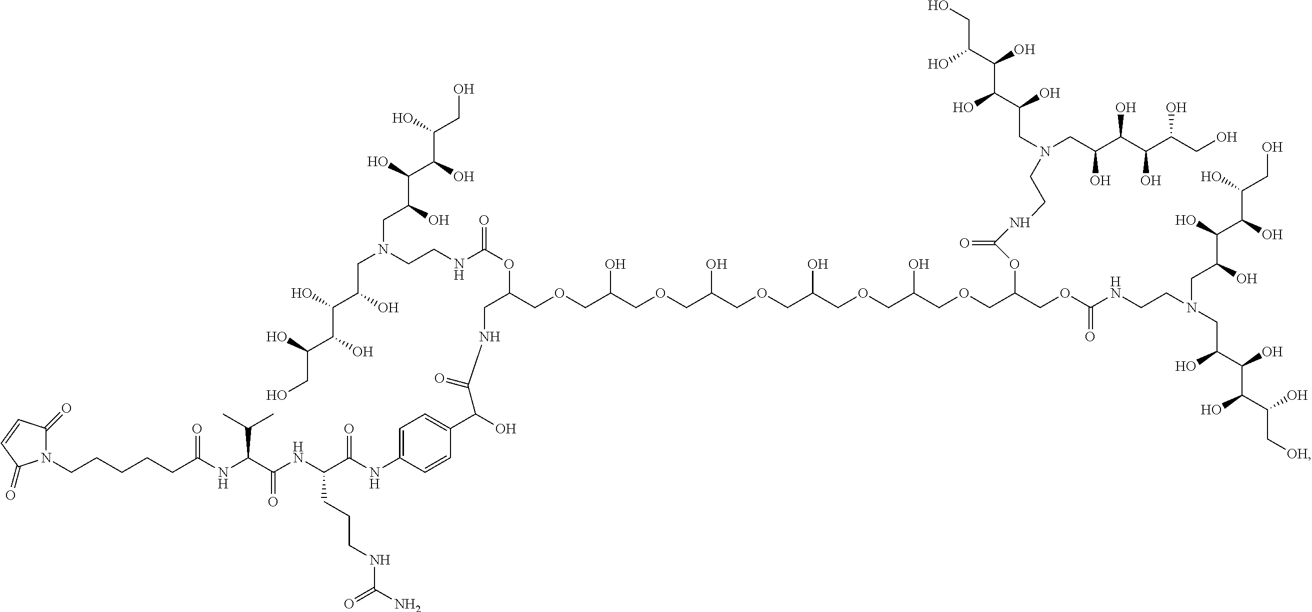 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCC(COCC(O)COCC(O)COCC(O)COCC(O)COCC(COC(=O)NCCN(C[C@H](O)[C@@H](O)[C@H](O)[C@H](O)CO)C[C@H](O)[C@@H](O)[C@H](O)[C@H](O)CO)OC(=O)NCCN(C[C@H](O)[C@@H](O)[C@H](O)[C@H](O)CO)C[C@H](O)[C@@H](O)[C@H](O)[C@H](O)CO)OC(=O)NCCN(C[C@H](O)[C@@H](O)[C@H](O)[C@H](O)CO)C[C@H](O)[C@@H](O)[C@H](O)[C@H](O)CO)cc1]

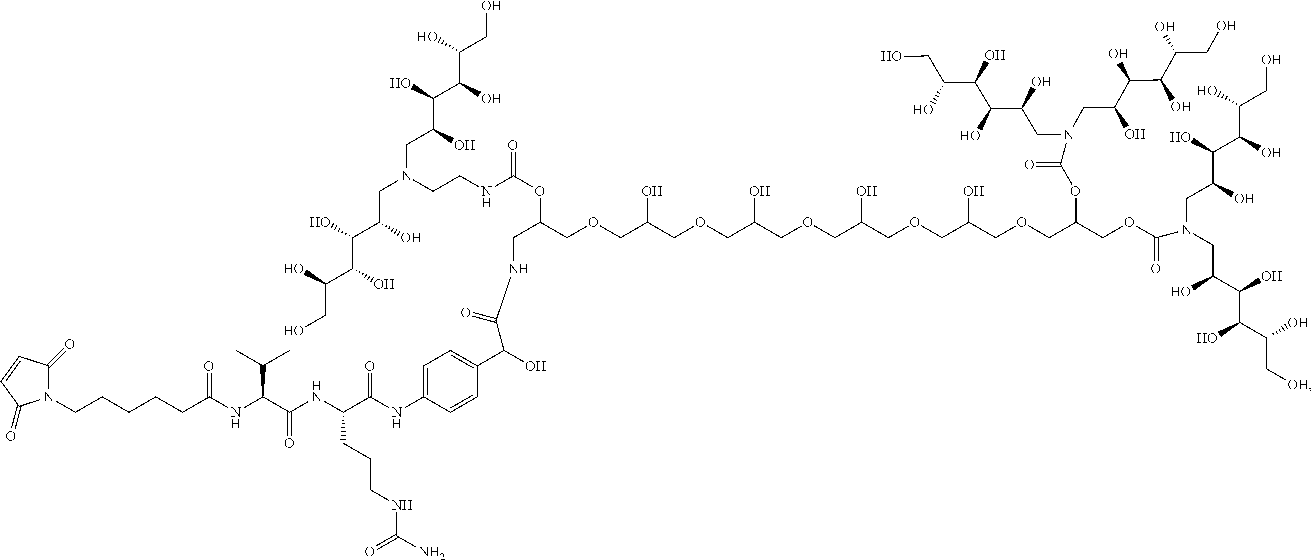 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCC(COCC(O)COCC(O)COCC(O)COCC(O)COCC(COC(=O)N(C[C@H](O)[C@@H](O)[C@H](O)[C@H](O)CO)C[C@H](O)[C@@H](O)[C@H](O)[C@H](O)CO)OC(=O)N(C[C@H](O)[C@@H](O)[C@H](O)[C@H](O)CO)C[C@H](O)[C@@H](O)[C@H](O)[C@H](O)CO)OC(=O)NCCN(C[C@H](O)[C@@H](O)[C@H](O)[C@H](O)CO)C[C@H](O)[C@@H](O)[C@H](O)[C@H](O)CO)cc1]

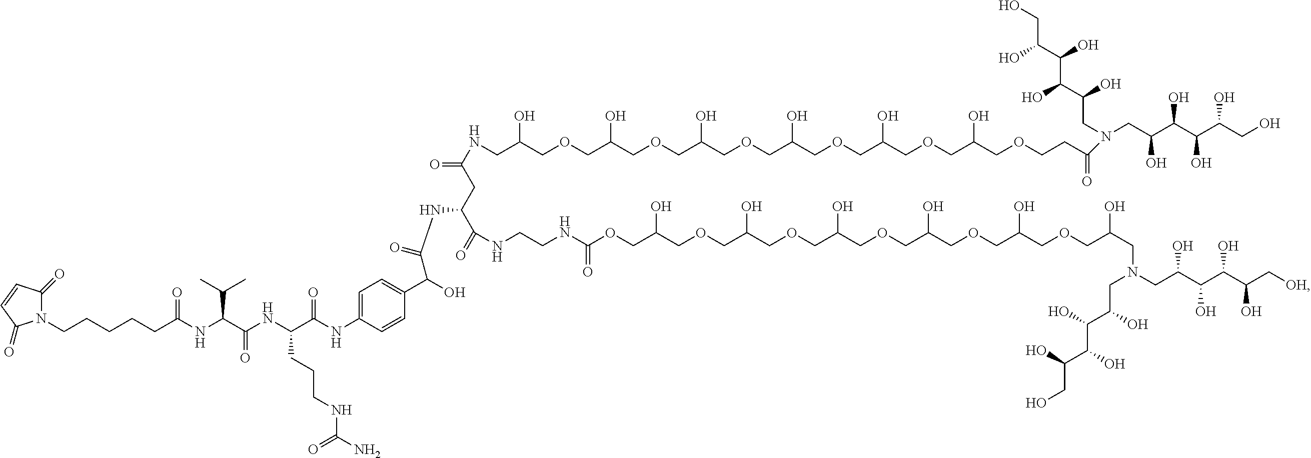 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCC(O)COCC(O)COCC(O)COCC(O)COCC(O)COCC(O)COCCC(=O)N(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)cc1]

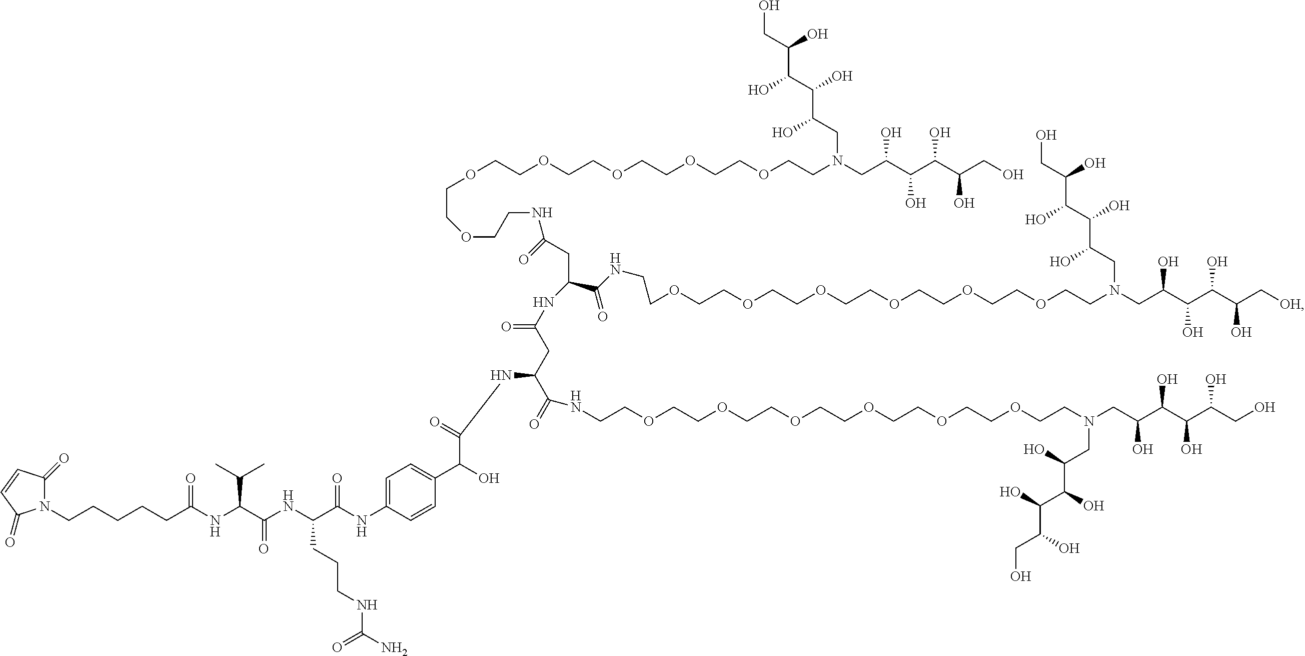 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@@H](CC(=O)N[C@@H](CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCN(C[C@@H](O)[C@@H](O)[C@H](O)[C@H](O)CO)C[C@H](O)[C@@H](O)[C@H](O)[C@H](O)CO)C(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

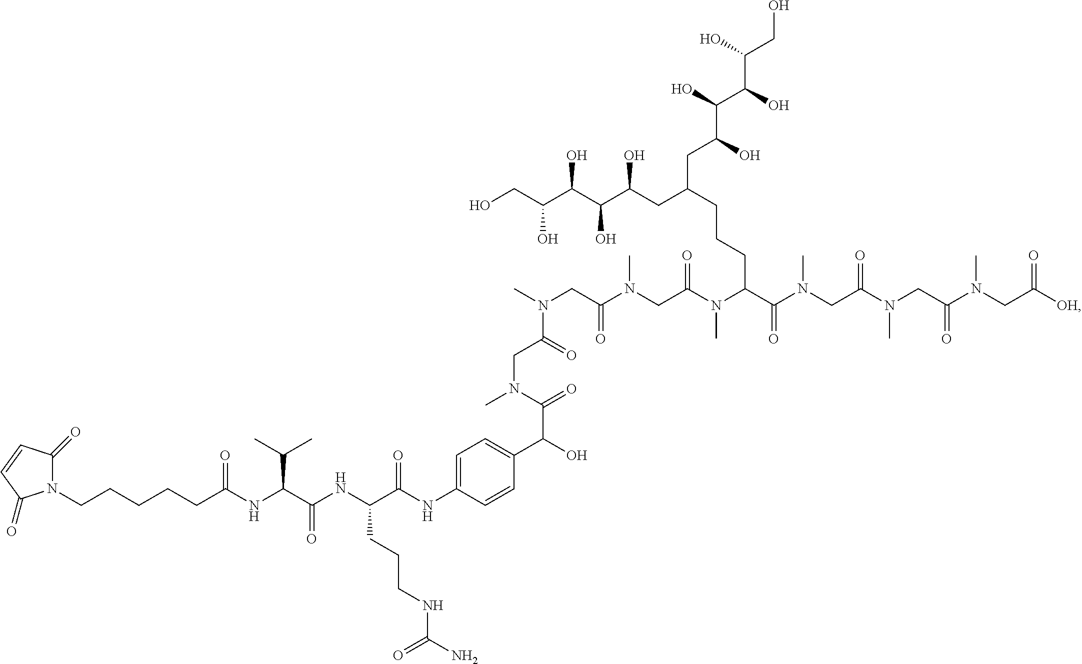 [SMILES: CC(C)[C@H](NC(=O)CCCCCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)C(CCCC(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)O)cc1]

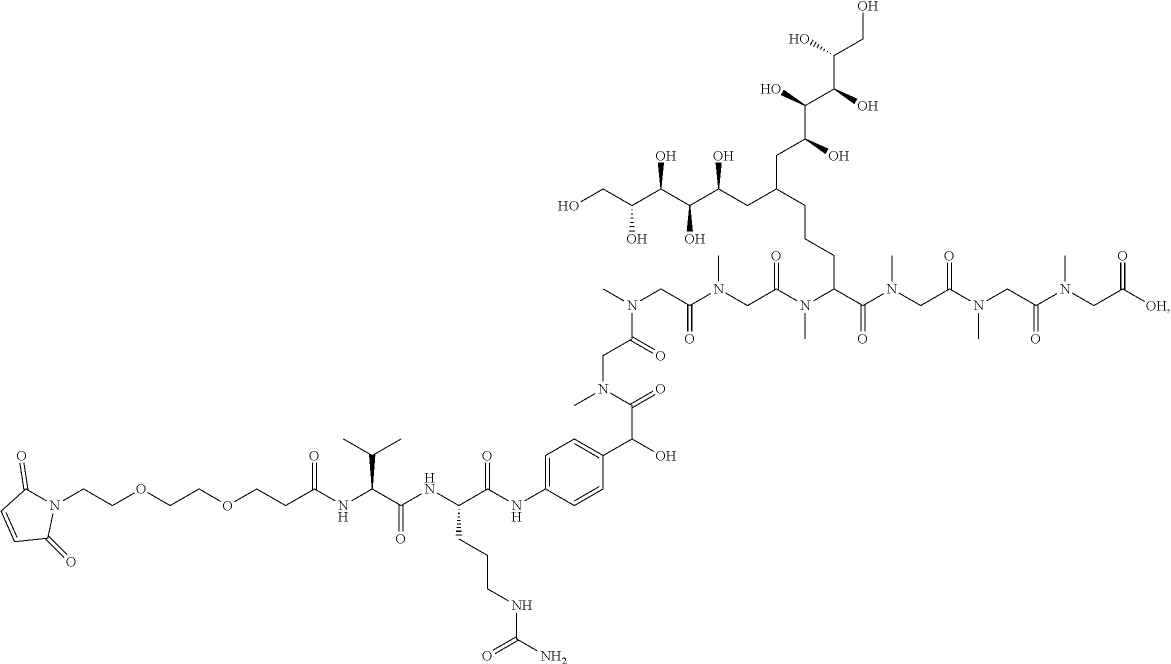 [SMILES: CC(C)[C@H](NC(=O)CCOCCOCCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)C(CCCC(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)O)cc1]

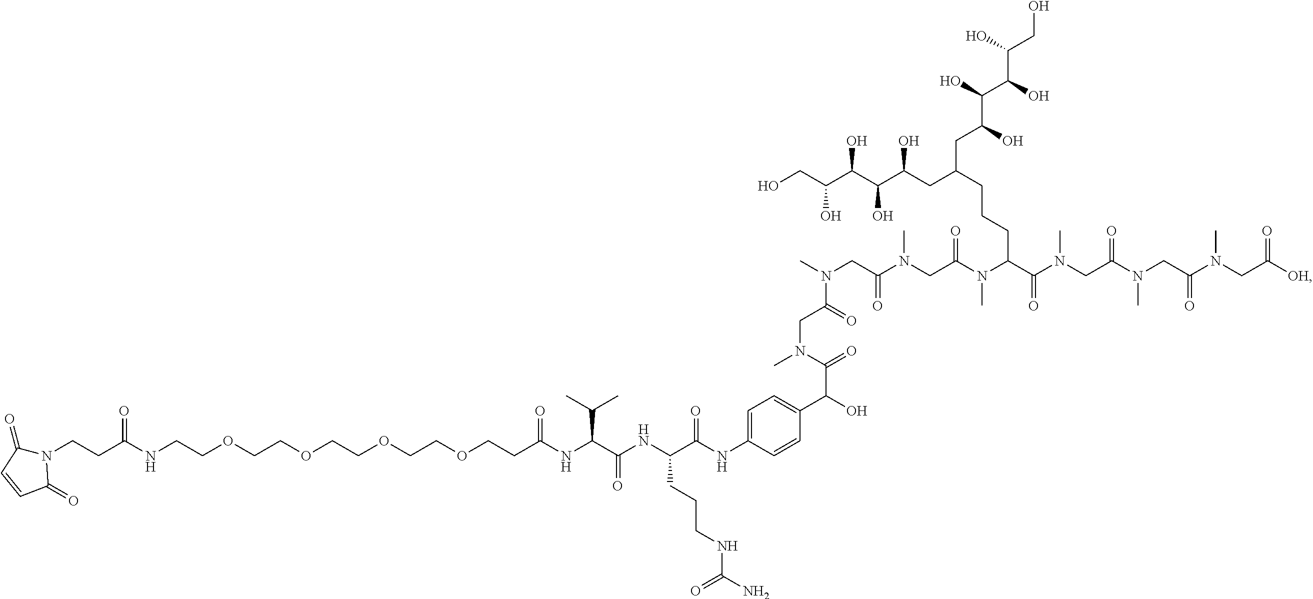 [SMILES: CC(C)[C@H](NC(=O)CCOCCOCCOCCOCCNC(=O)CCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)C(CCCC(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)O)cc1]

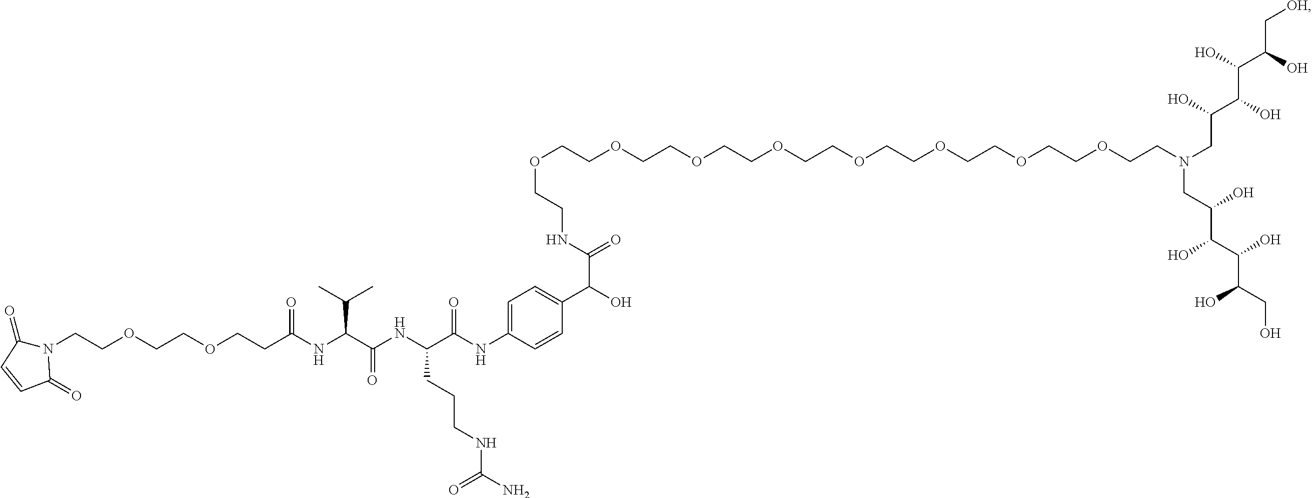 [SMILES: CC(C)[C@H](NC(=O)CCOCCOCCN1C(=O)C=CC1=O)C(=O)N[C@@H](CCCNC(N)=O)C(=O)Nc1ccc(C(O)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

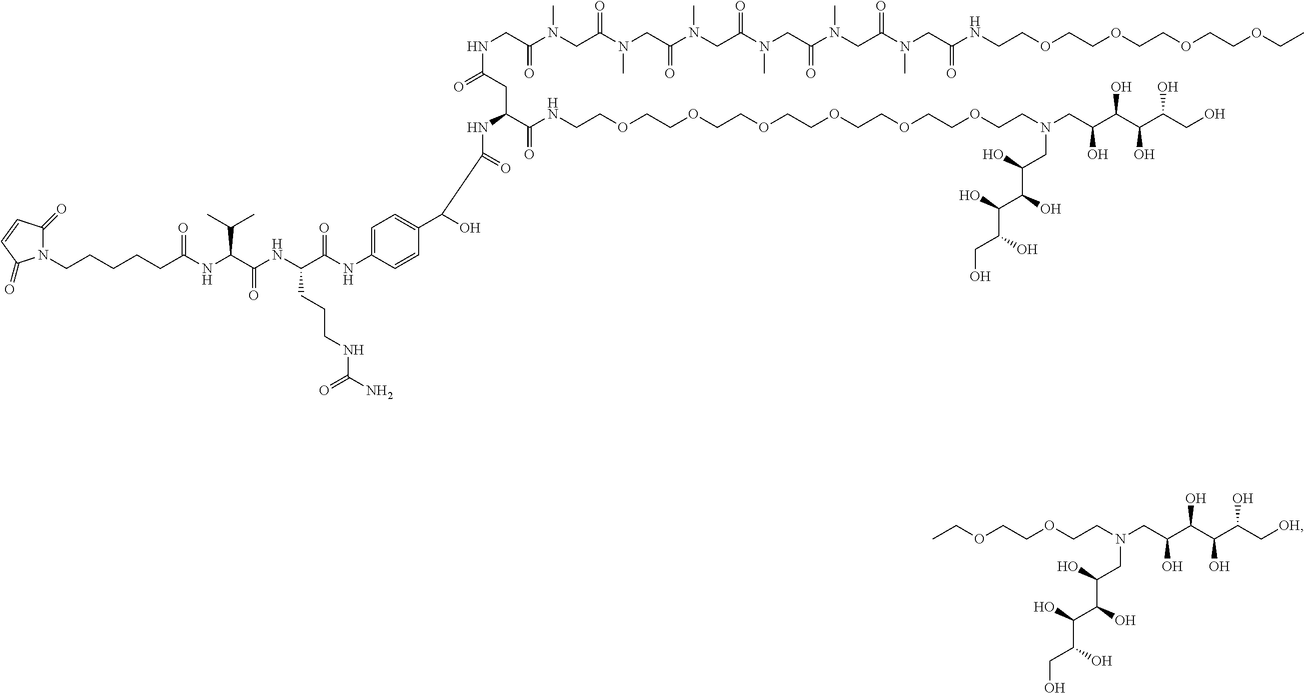

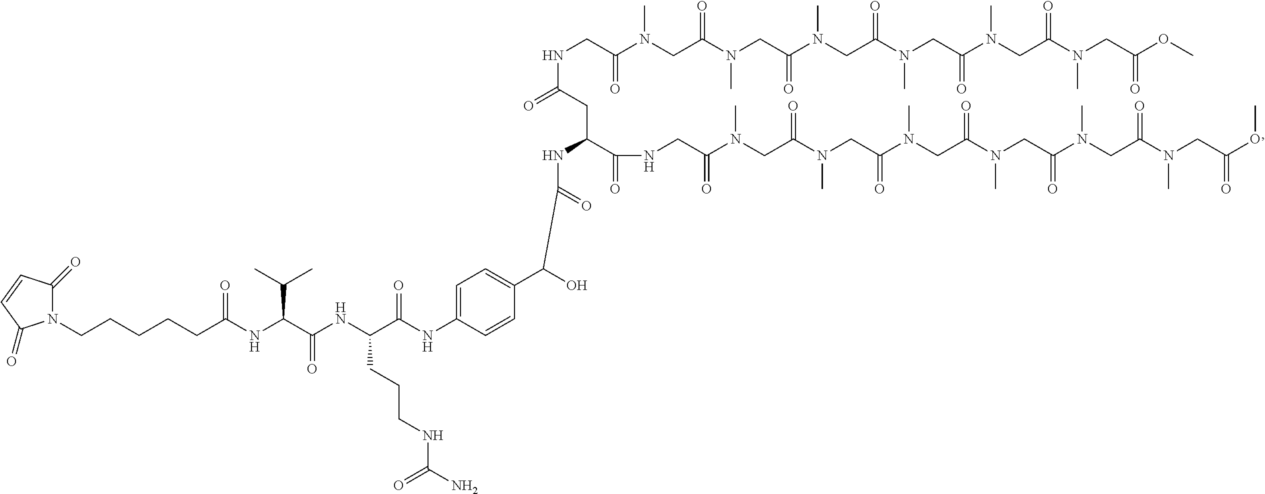 [SMILES: COC(=O)CN(C)C(=O)CN(C)C(=O)CN(C)C(=O)CN(C)C(=O)CN(C)C(=O)CN(C)C(=O)CNC(=O)C[C@H](NC(=O)C(O)c1ccc(NC(=O)[C@H](CCCNC(N)=O)NC(=O)[C@@H](NC(=O)CCCCCN2C(=O)C=CC2=O)C(C)C)cc1)C(=O)NCC(=O)N(C)CC(=O)N(C)CC(=O)N(C)CC(=O)N(C)CC(=O)N(C)CC(=O)N(C)CC(=O)OC]

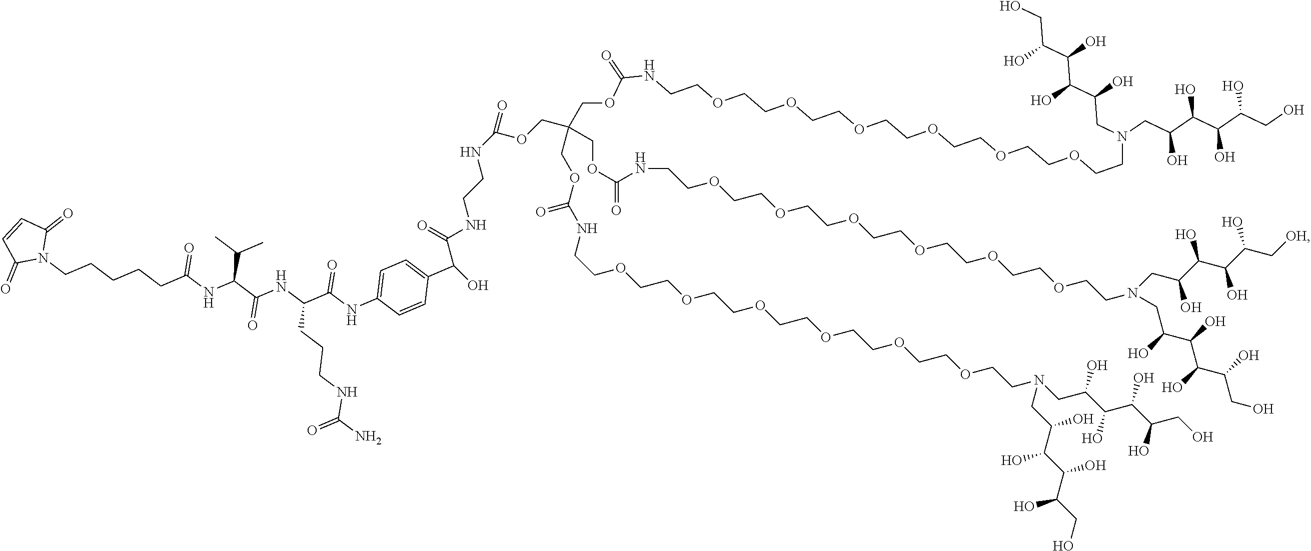 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCCNC(=O)OCC(COC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)(COC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)COC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

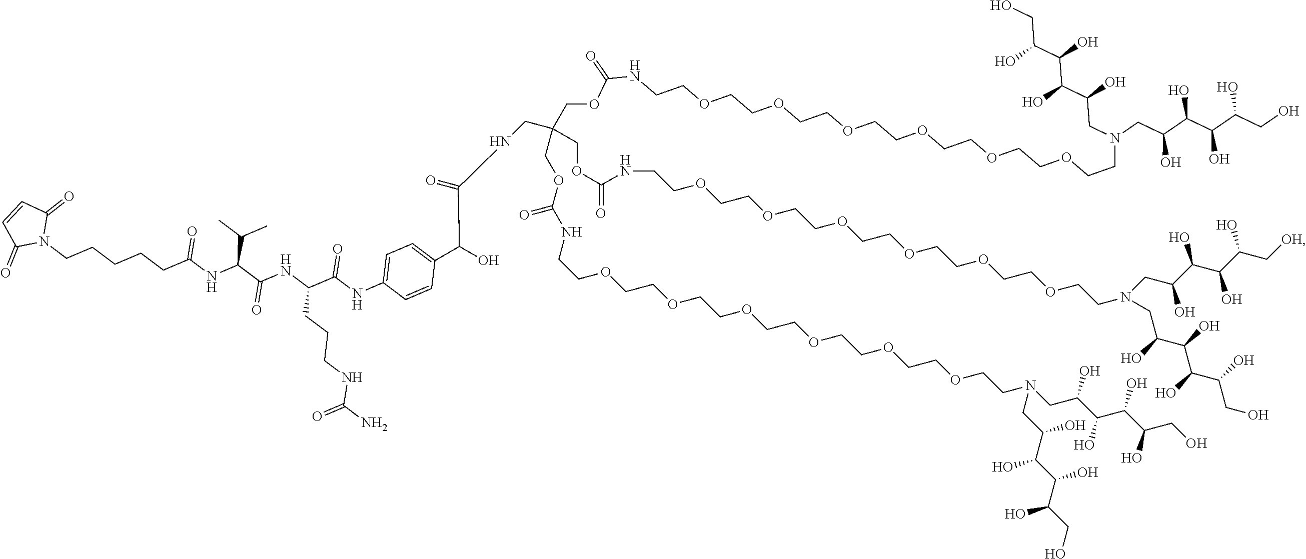 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCC(COC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)(COC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)COC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

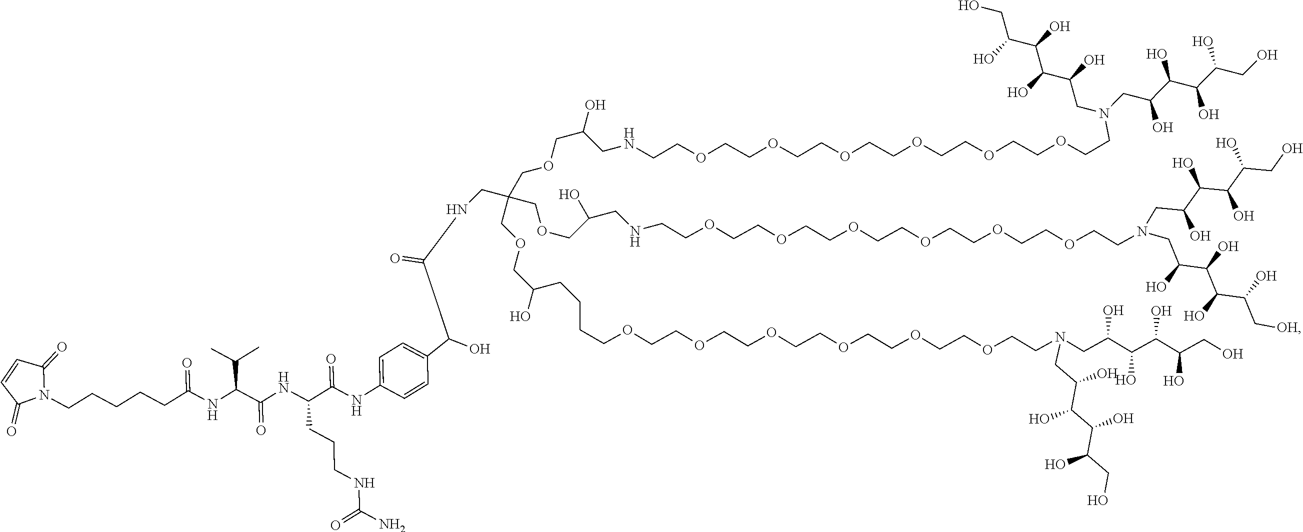 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCC(COCC(O)CCCCOCCOCCOCCOCCOCCOCCN(C[C@H](O)[C@@H](O)[C@H](O)[C@H](O)CO)C[C@H](O)[C@@H](O)[C@H](O)[C@H](O)CO)(COCC(O)CNCCOCCOCCOCCOCCOCCOCCN(C[C@H](O)[C@@H](O)[C@H](O)[C@H](O)CO)C[C@H](O)[C@@H](O)[C@H](O)[C@H](O)CO)COCC(O)CNCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

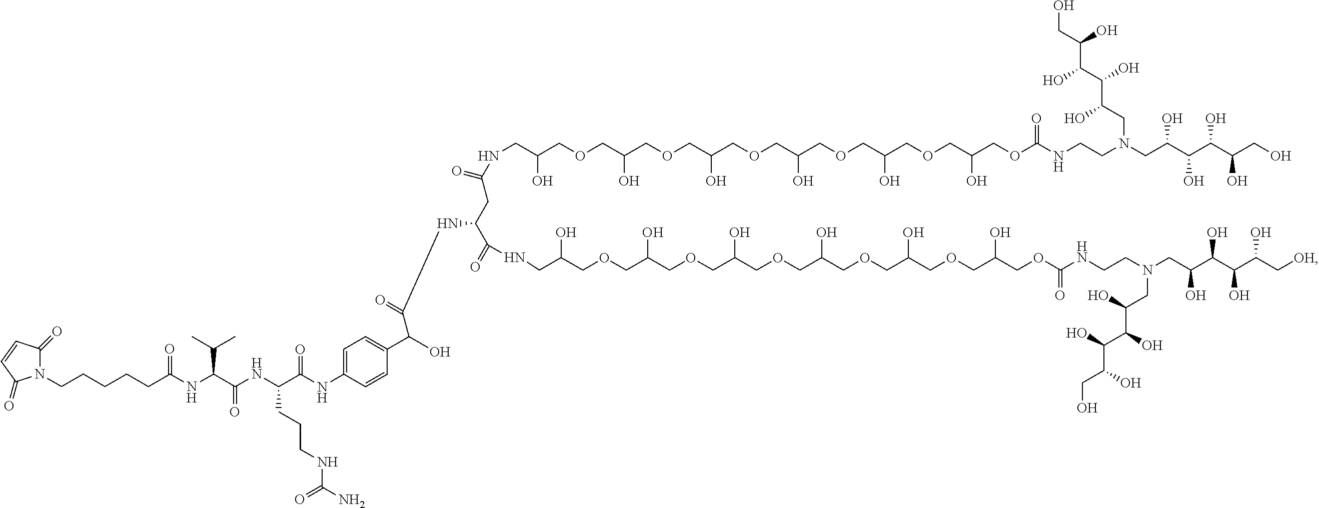 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCC(O)COCC(O)COCC(O)COCC(O)COCC(O)COCC(O)COC(=O)NCCN(C[C@H](O)[C@@H](O)[C@H](O)[C@H](O)CO)C[C@H](O)[C@@H](O)[C@H](O)[C@H](O)CO)C(=O)NCC(O)COCC(O)COCC(O)COCC(O)COCC(O)COCC(O)COC(=O)NCCN(C[C@H](O)[C@@H](O)[C@H](O)[C@H](O)CO)C[C@H](O)[C@@H](O)[C@H](O)[C@H](O)CO)cc1]

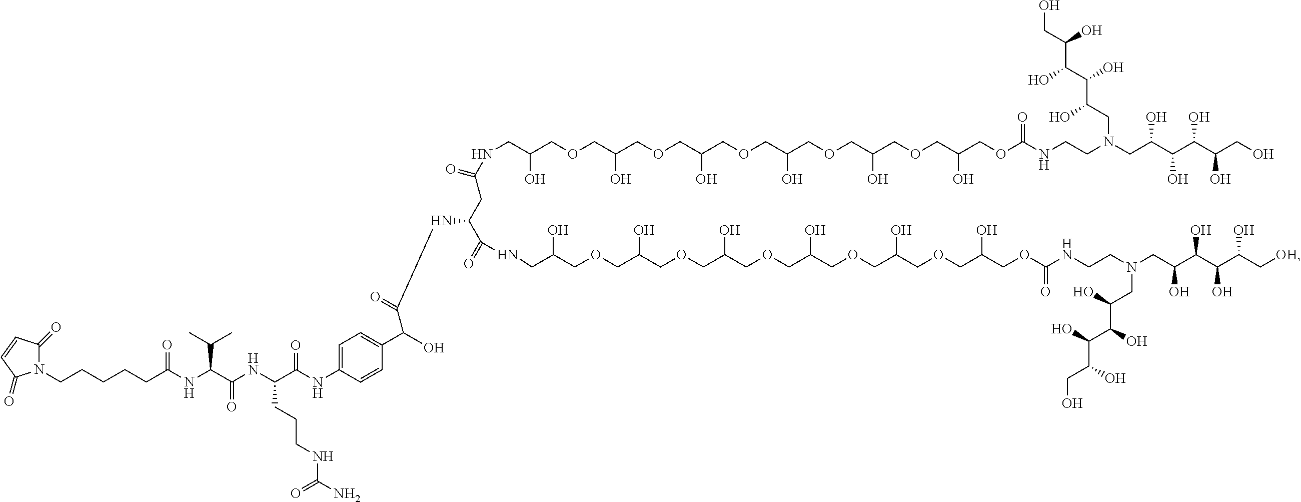 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCC(O)COCC(O)COCC(O)COCC(O)COCC(O)COCC(O)COC(=O)NCCN(C[C@H](O)[C@@H](O)[C@H](O)[C@H](O)CO)C[C@H](O)[C@@H](O)[C@H](O)[C@H](O)CO)C(=O)NCC(O)COCC(O)COCC(O)COCC(O)COCC(O)COCC(O)COC(=O)NCCN(C[C@H](O)[C@@H](O)[C@H](O)[C@H](O)CO)C[C@H](O)[C@@H](O)[C@H](O)[C@H](O)CO)cc1]

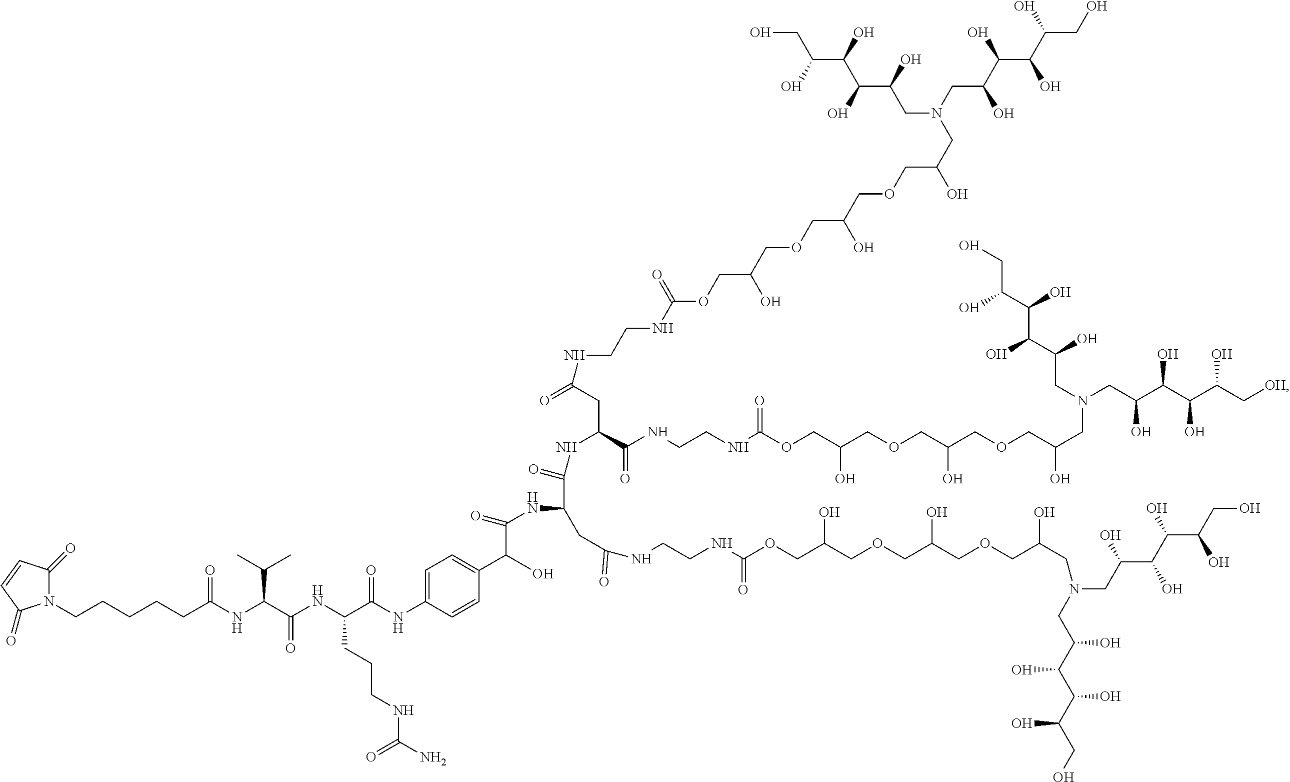 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)N[C@@H](CC(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)cc1]

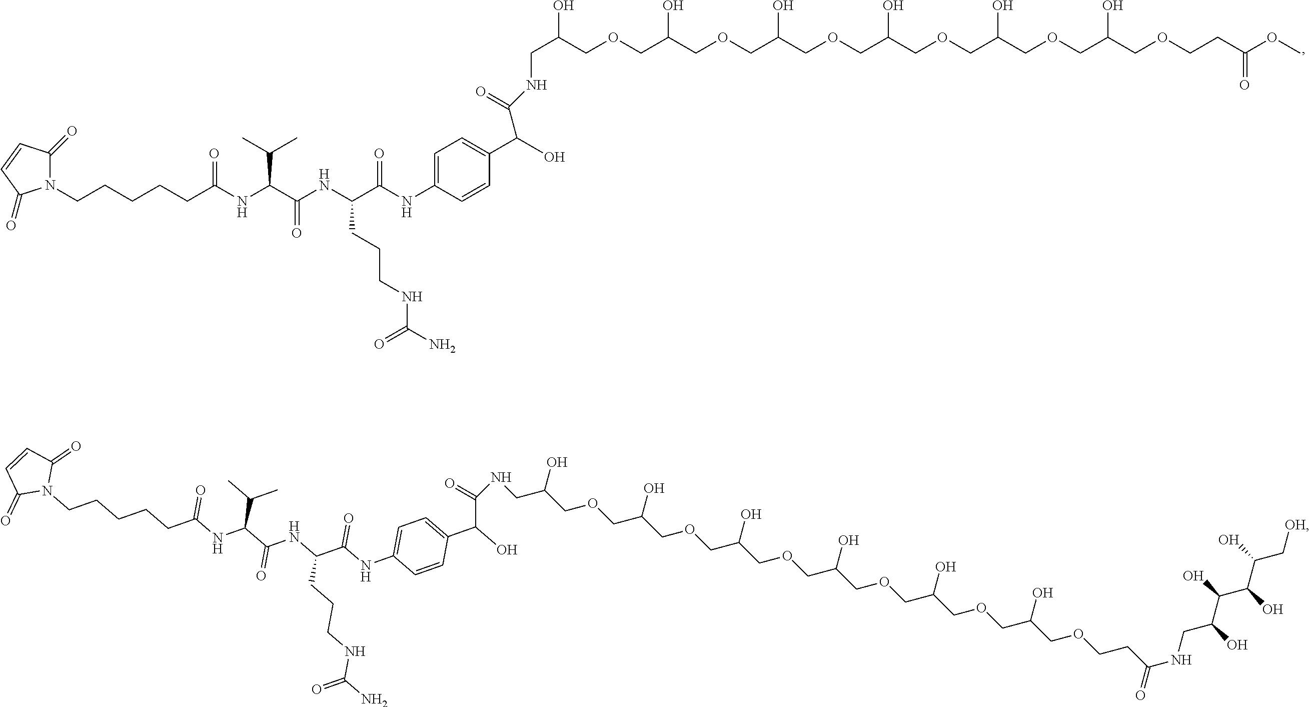 [SMILES: COC(=O)CCOCC(O)COCC(O)COCC(O)COCC(O)COCC(O)COCC(O)CNC(=O)C(O)c1ccc(NC(=O)[C@H](CCCNC(N)=O)NC(=O)[C@@H](NC(=O)CCCCCN2C(=O)C=CC2=O)C(C)C)cc1 ; CC(C)[C@H](NC(=O)CCCCCN1C(=O)C=CC1=O)C(=O)N[C@@H](CCCNC(N)=O)C(=O)Nc1ccc(C(O)C(=O)NCC(O)COCC(O)COCC(O)COCC(O)COCC(O)COCC(O)COCCC(=O)NC[C@H](O)[C@@H](O)[C@H](O)[C@H](O)CO)cc1]

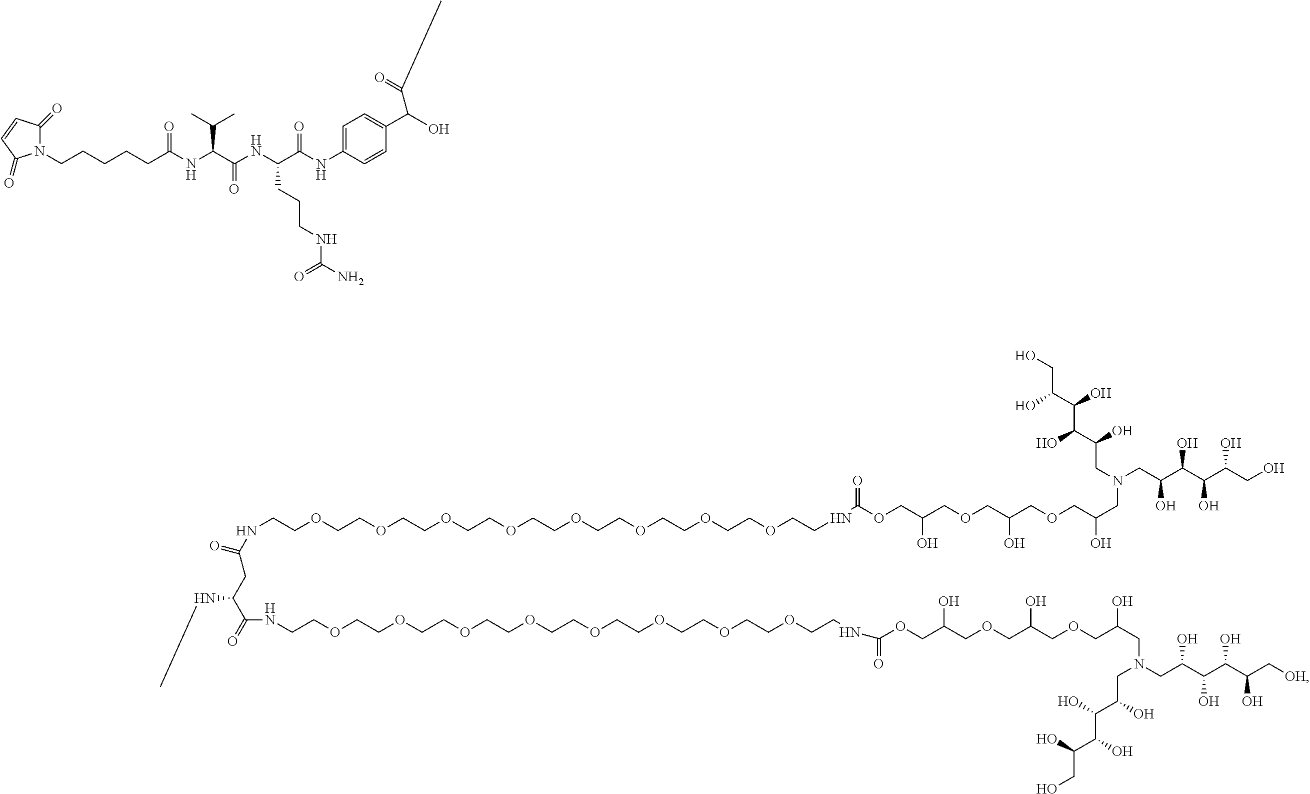 [SMILES: CC(=O)C(O)c1ccc(NC(=O)[C@H](CCCNC(N)=O)NC(=O)[C@@H](NC(=O)CCCCCN2C(=O)C=CC2=O)C(C)C)cc1 ; CN[C@H](CC(=O)NCCOCCOCCOCCOCCOCCOCCOCCOCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO]

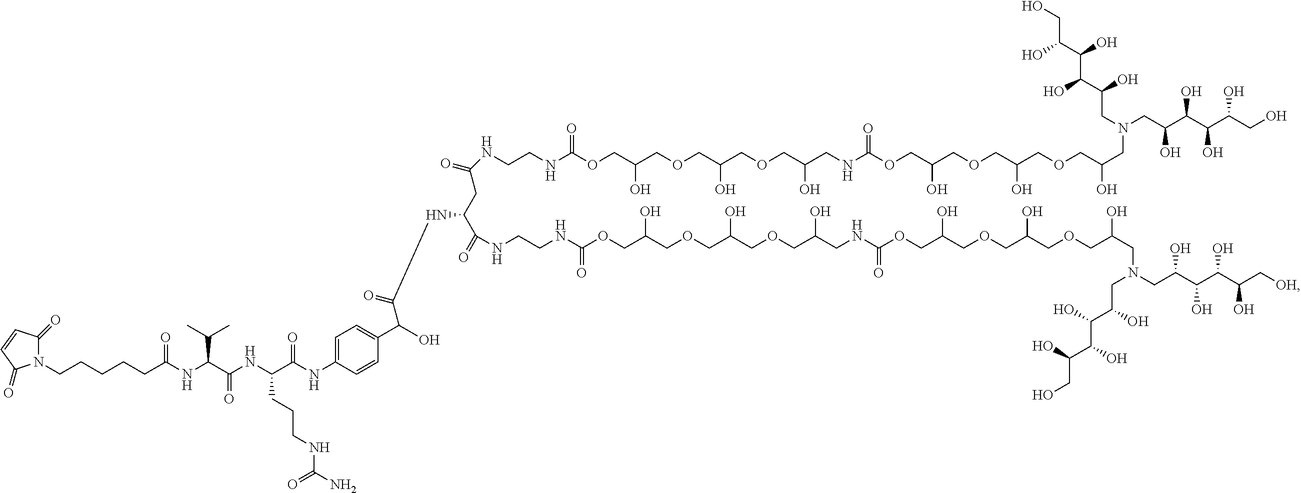 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCCNC(=O)OCC(O)COCC(O)COCC(O)CNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)CNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)cc1]

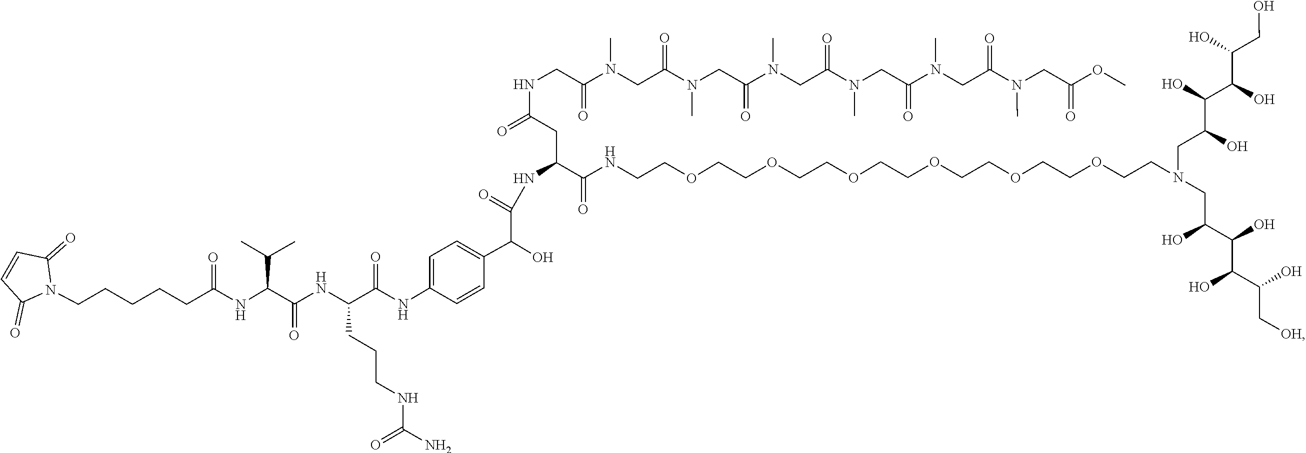 [SMILES: COC(=O)CN(C)C(=O)CN(C)C(=O)CN(C)C(=O)CN(C)C(=O)CN(C)C(=O)CN(C)C(=O)CNC(=O)C[C@H](NC(=O)C(O)c1ccc(NC(=O)[C@H](CCCNC(N)=O)NC(=O)[C@@H](NC(=O)CCCCCN2C(=O)C=CC2=O)C(C)C)cc1)C(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO]

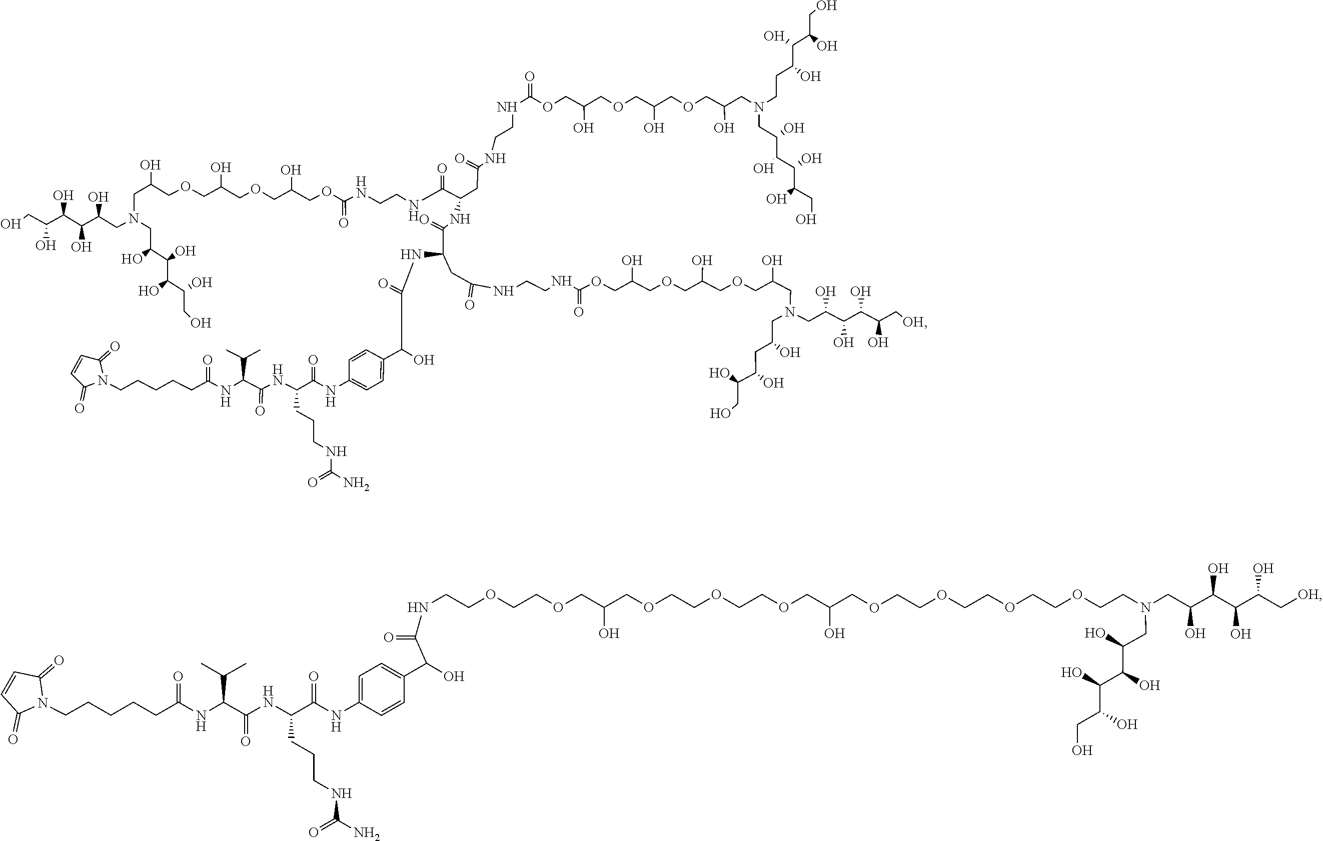

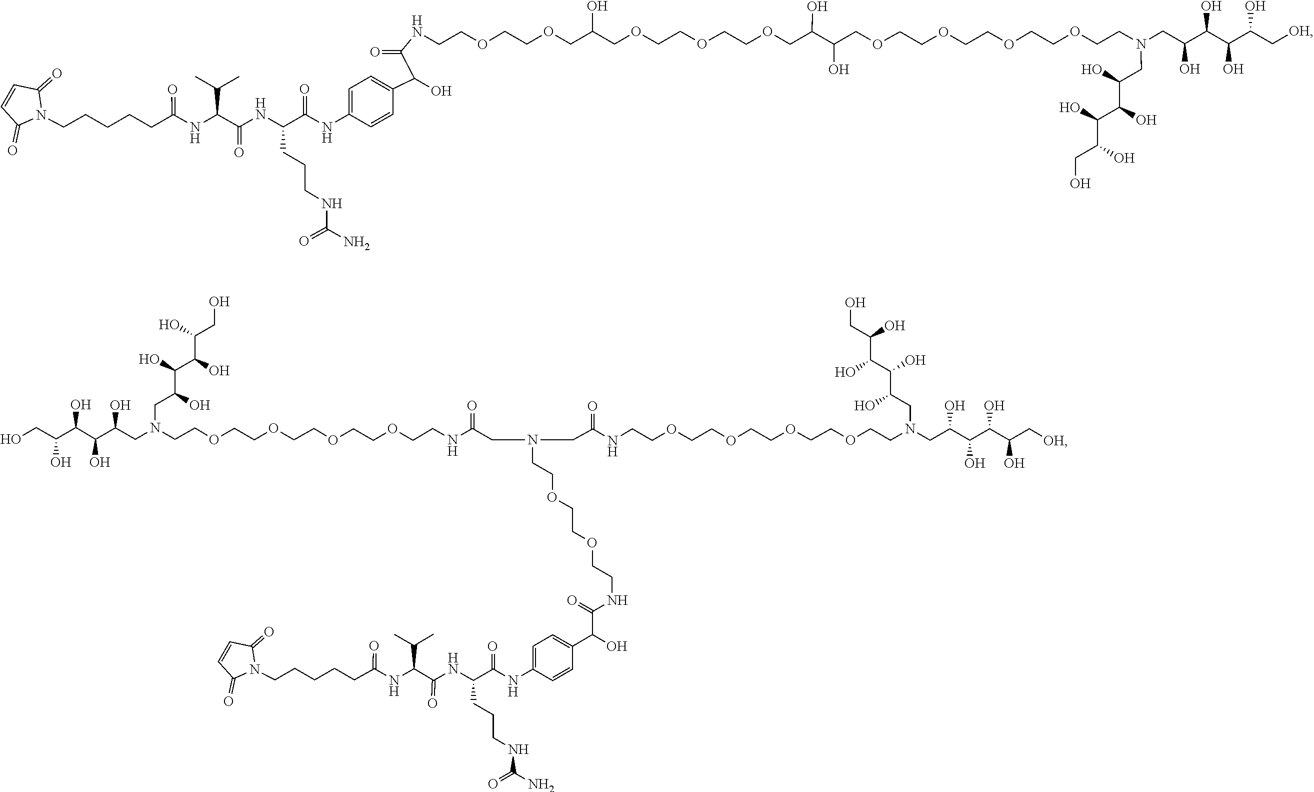

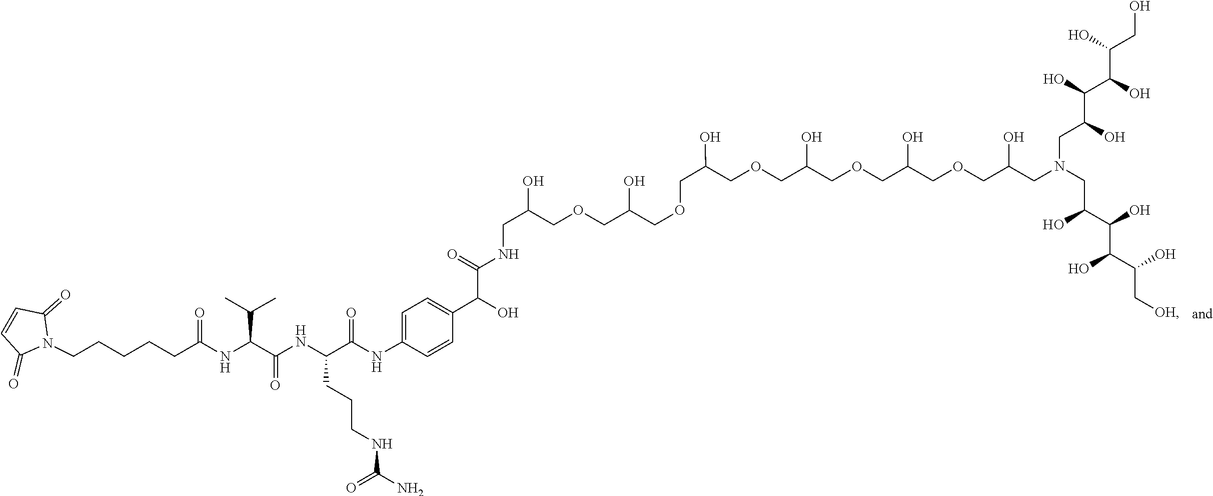

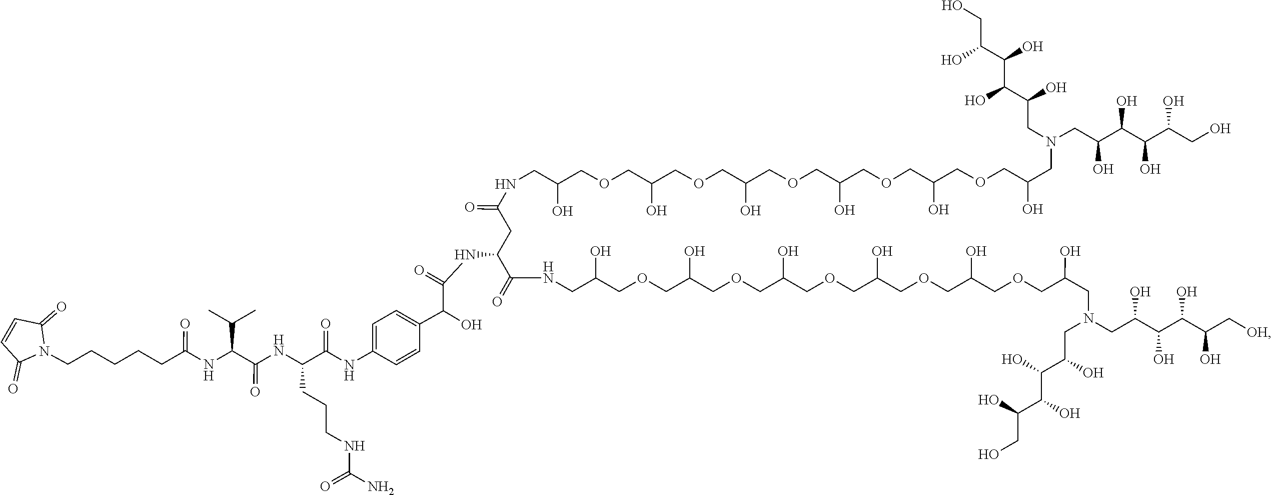 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)cc1]

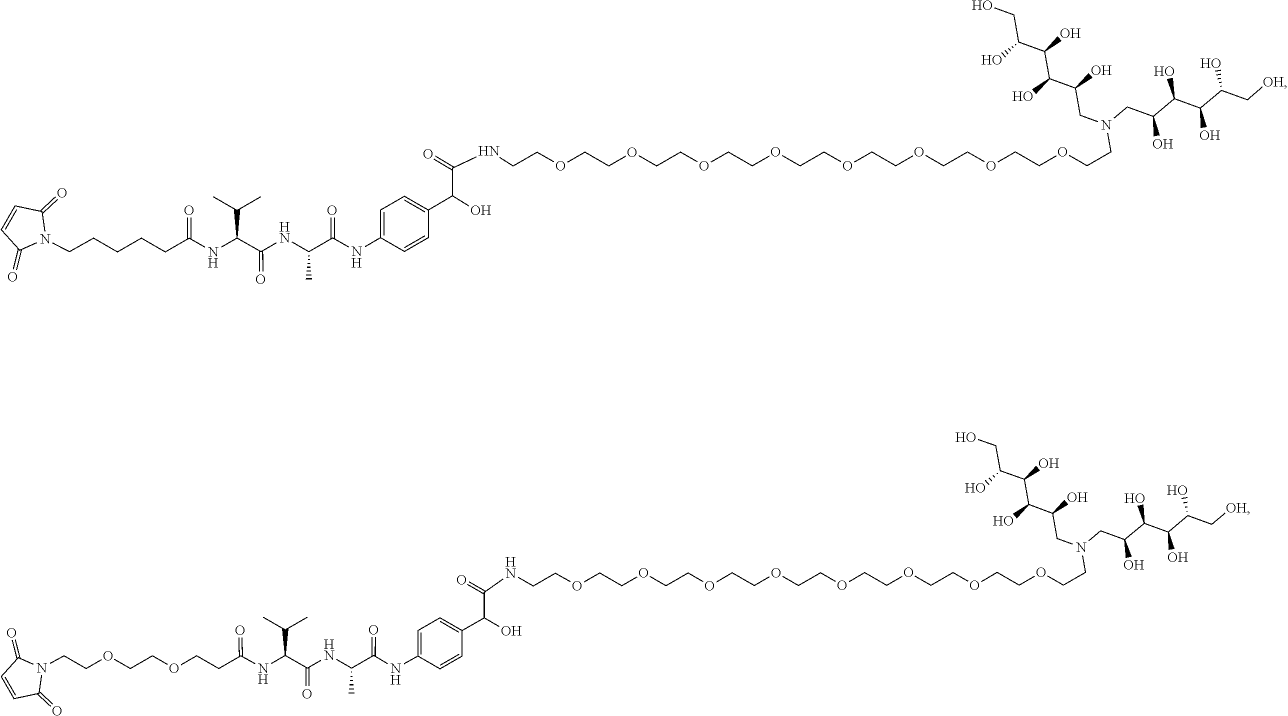 [SMILES: CC(C)[C@H](NC(=O)CCCCCN1C(=O)C=CC1=O)C(=O)N[C@@H](C)C(=O)Nc1ccc(C(O)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1 ; CC(C)[C@H](NC(=O)CCOCCOCCN1C(=O)C=CC1=O)C(=O)N[C@@H](C)C(=O)Nc1ccc(C(O)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

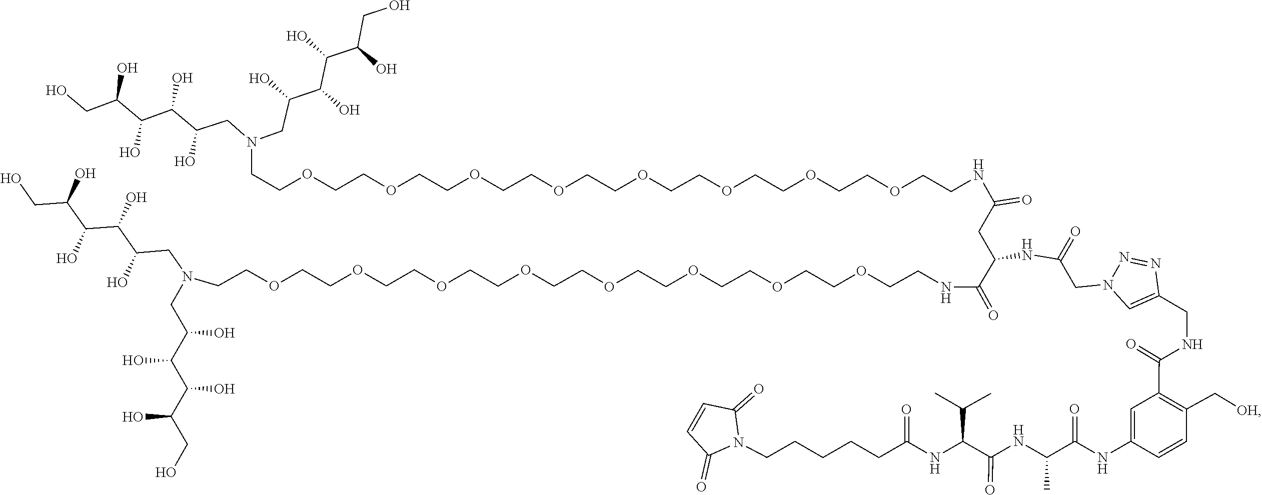 [SMILES: CC(C)[C@H](NC(=O)CCCCCN1C(=O)C=CC1=O)C(=O)N[C@@H](C)C(=O)Nc1ccc(CO)c(C(=O)NCc2cn(CC(=O)N[C@@H](CC(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)nn2)c1]

wherein the H on the benzylic OH is optionally replaced with a bond to the at least one Drug unit or to the linking group attached to the at least one Drug unit.

In some embodiments, provided is a Drug-Linker compound, comprising a Linker compound as described herein attached to the at least one Drug unit, or attached to the linking group attached to the at least one Drug unit, at the attachment site.

### Drug Units

In some embodiments, the Linkers are attached to at least one Drug unit. As used herein, the term “Drug unit” or drug refers to cytotoxic agents (such as chemotherapeutic agents or drugs), immunomodulatory agents, nucleic acids (including siRNAs), growth inhibitory agents, toxins (e.g., protein toxins, enzymatically active toxins of bacterial, fungal, plant, or animal origin, or fragments thereof), radioactive isotopes, PROTACs and other compounds that are active against target cells when delivered to those cells.

In some embodiments, provided is a Drug-Linker wherein the Drug unit is selected from a cytotoxic agent, an immune modulatory agent, a nucleic acid, a growth inhibitory agent, a PROTAC, a toxin, a radioactive isotope and a chelating ligand.

### Cytotoxic Agents

In some embodiments, a Drug unit is a cytotoxic agent. A “cytotoxic agent” refers to an agent that has a cytotoxic effect on a cell. A “cytotoxic effect” refers to the depletion, elimination and/or the killing of a target cell(s). Cytotoxic agents include, for example, tubulin disrupting agents, topoisomerase inhibitors, DNA minor groove binders, and DNA alkylating agents.

In some embodiments, the cytotoxic agent is selected from the group consisting of an auristatin, a maytansinoid, a camptothecin, a duocarmycin, and a calicheamicin. In some embodiments, the cytotoxic agent is an auristatin. Oth other embodiments, the cytotoxic agent is MMAE or MMAF. In some embodiments, the cytotoxic agent is a camptothecin. In some embodiments, the cytotoxic agent is exatecan or SN-38 or DxD. In certain embodiments, the cytotoxic agent is RS-exatecan or SS-exatecan. In other embodiments, the cytotoxic agent is a calicheamicin. In some embodiments, the cytotoxic agent is a maytansinoid. In certain embodiments, the maytansinoid is maytansine, maytansinol or ansamatocin-2.

Tubulin disrupting agents include, for example, auristatins, dolastatins, tubulysins, colchicines, vinca alkaloids, taxanes, cryptophycins, maytansinoids, hemiasterlins, as well as other tubulin disrupting agents. Auristatins are derivatives of the natural product dolastatin 10. Exemplary auristatins include MMAE (N-methylvaline-valine-dolaisoleuine-dolaproine-norephedrine), MMAF (N-methylvaline-valine-dolaisoleuine-dolaproine-phenylalanine) and AFP (see WO2004/010957 and WO2007/008603). Other auristatin like compounds are disclosed in, for example, Published US Application Nos. US2021/0008099, US2017/0121282, US2013/0309192 and US2013/0157960. Dolastatins include, for example, dolastatin 10 and dolastatin 15 (see, e.g., Pettit et al., J. Am. Chem. Soc., 1987, 109, 6883-6885; Pettit et al., Anti-Cancer Drug Des., 1998, 13, 243-277; and Published US Application US2001/0018422). Additional dolastatin derivatives contemplated for use herein are disclosed in U.S. Pat. No. 9,345,785, incorporated herein by reference.

Tubulysins include, but are not limited to, tubulysin D, tubulysin M, tubuphenylalanine and tubutyrosine. WO2017/096311 and WO/2016-040684 describe tubulysin analogs including tubulysin M.

Colchicines include, but are not limited to, colchicine and CA-4.

Vinca alkaloids include, but are not limited to, vinblastine (VBL), vinorelbine (VRL), vincristine (VCR) and vindesine (VOS).

Taxanes include, but are not limited to, paclitaxel and docetaxel.

Cryptophycins include but are not limited to cryptophycin-1 and cryptophycin-52.

Maytansinoids include, but are not limited to, maytansine, maytansinol, maytansine analogs in DM1, DM3 and DM4, and ansamatocin-2. Exemplary maytansinoid drug moieties include those having a modified aromatic ring, such as: C-19-dechloro (U.S. Pat. No. 4,256,746) (prepared by lithium aluminum hydride reduction of ansamitocin P2); C-20-hydroxy (or C-20-demethyl)+/−C-19-dechloro (U.S. Pat. Nos. 4,361,650 and 4,307,016) (prepared by demethylation using Streptomyces or Actinomyces or dechlorination using LAH); and C-20- demethoxy, C-20-acyloxy (—OCOR), +/−dechloro (U.S. Pat. No. 4,294,757) (prepared by acylation using acyl chlorides), and those having modifications at other positions.

Maytansinoid drug moieties also include those having modifications such as: C-9-SH (U.S. Pat. No. 4,424,219) (prepared by the reaction of maytansinol with H₂S or P₂S₅); C-14-alkoxymethyl(demethoxy/CH₂OR) (see, U.S. Pat. No. 4,331,598); C-14- hydroxymethyl or acyloxymethyl (CH₂OH or CH₂OAc) (see, U.S. Pat. No. 4,450,254) (prepared from Nocardia); C-15-hydroxy/acyloxy (see, U.S. Pat. No. 4,364,866) (prepared by the conversion of maytansinol by Streptomyces); C-15-methoxy (see, U.S. Pat. Nos. 4,313,946 and 4,315,929) (isolated from Trewia nudiflora); C-18-N-demethyl (see, U.S. Pat. Nos. 4,362,663 and 4,322,348) (prepared by the demethylation of maytansinol by Streptomyces); and 4,5-deoxy (see, U.S. Pat. No. 4,371,533) (prepared by the titanium trichloride/LAH reduction of maytansinol).

Hemiasterlins include but are not limited to, hemiasterlin and HTI-286.

Other tubulin disrupting agents include taccalonolide A, taccalonolide B, taccalonolide AF, taccalonolide AJ, taccalonolide AI-epoxide, discodermolide, epothilone A, epothilone B, and laulimalide.

In some embodiments, a cytotoxic agent can be a topoisomerase inhibitor, such as a camptothecin. Exemplary camptothecins include, for example, camptothecin, irinotecan (also referred to as CPT-11), belotecan, (7-(2-(N-isopropylamino)ethyl)camptothecin), topotecan, 10-hydroxy-CPT, SN-38, exatecan and the exatecan analog DXd (see US20150297748). In some embodiments, the cytotoxic agent is exatecan or SN-38 or DxD. In some embodiments, the cytotoxic agent is RS exatecan or SS exatecan. Other camptothecins are disclosed in WO1996/021666, WO00/08033, US2016/0229862 and WO2020/156189.

In some embodiments, a cytotoxic agent is a duocarmcycin, including the synthetic analogues, KW-2189 and CBI-TMI.

### Immune Modulatory Agents

In some embodiments, a Drug unit is an immune modulatory agent. An immune modulatory agent can be, for example, a TLR7 and/or TLR8 agonist, a STING agonist, a RIG-1 agonist or other immune modulatory agent. In some embodiments, the immune modulatory agent is selected from a TRL7 agonist, a TLR8 agonist, a STING agonist, or a RIG-1 agonist.

In some embodiments, the immune modulatory agent is an TLR7 agonist. In certain embodiments, the TLR7 agonist is an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, a benzonaphthyridine, a guanosine analog, an adenosine analog, a thymidine homopolymer, ssRNA, CpG-A, PolyG10, or PolyG3. In some embodiments, the immune modulatory agent is a TLR8 agonist. In certain embodiments, the TLR8 agonist is selected from an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine or a ssRNA. In some embodiments, the immune modulatory agent is a STING agonist. In other embodiments, the immune modulatory agent is a RIG-1 agonist. In certain embodiments, the RIG-1 agonist is selected from KIN1148, SB-9200, KIN700, KIN600, KIN500, KIN100, KIN101, KIN400 and KIN2000.

In some embodiments, a Drug unit is an immune modulatory agent, such as a TLR7 and/or TLR8 agonist. In some embodiments, a TLR7 agonist is selected from an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, a benzonaphthyridine, a guanosine analog, an adenosine analog, a thymidine homopolymer, ssRNA, CpG-A, PolyG10, and PolyG3. In some embodiments, the TLR7 agonist is selected from an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide or a benzonaphthyridine. In some embodiments, a TLR7 agonist is a non-naturally occurring compound. Examples of TLR7 modulators include GS-9620, GSK-2245035, imiquimod, resiquimod, DSR-6434, DSP-3025, IMO-4200, MCT-465, MEDI-9197, 3M-051, SB-9922, 3M-052, Limtop, TMX-30X, TMX-202, RG- 7863, RG-7795, and the compounds disclosed in US20160168164, US 20150299194, US20110098248, US20100143301, and US20090047249.

In some embodiments, a TLR8 agonist is selected from a benzazepine, an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine or a ssRNA. In some embodiments, a TLR8 agonist is selected from a benzazepine, an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, and a tetrahydropyrido pyrimidine. In some embodiments, a TLR8 agonist is a non-naturally occurring compound. Examples of TLR8 agonists include motolimod, resiquimod, 3M-051, 3M-052, MCT-465, IMO-4200, VTX-763, VTX-1463.

In some embodiments, a TLR8 agonist can be any of the compounds described WO2018/170179, WO2020/056198 and WO2020056194.

Other TLR7 and TLR8 agonists are disclosed in, for example, WO2016142250, WO2017046112, WO2007024612, WO2011022508, WO2011022509, WO2012045090, WO2012097173, WO2012097177, WO2017079283, US20160008374, US20160194350, US20160289229, U.S. Pat. No. 6,043,238, US20180086755, WO2017216054, WO2017190669, WO2017202704, WO2017202703, WO20170071944, US20140045849, US20140073642, WO2014056953, WO2014076221, WO2014128189, US20140350031, WO2014023813, US20080234251, US20080306050, US20100029585, US20110092485, US20110118235, US20120082658, US20120219615, US20140066432, US20140088085, US20140275167, and US20130251673, WO2018198091, and US20170131421.

In some embodiments, an immune modulatory agent is a STING agonist. Examples of STING agonists include, for example, those disclosed in WO2020059895, WO2015077354, WO2020227159, WO2020075790, WO2018200812, and WO2020074004.

In some embodiments, an immune modulatory agent is a RIG-1 agonist. Examples of RIG-1 agonists include KIN1148, SB-9200, KIN700, KIN600, KIN500, KIN100, KIN101, KIN400 and KIN2000.

### Toxins

In some embodiments, a Drug unit is an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

### Radioisotopes

In some embodiments, a Drug unit is a radioactive atom. A variety of radioactive isotopes are available for the production of radioconjugates. Examples include yittrium-88, yittrium-90, technetium-99, copper-67, rhenium-188, rhenium-186, galium-66, galium-67, indium-111, indium-114, indium-115, lutetium-177, strontium-89, sararium-153, and lead-212.

### PROTACs

In some embodiments, a Drug unit is a proteolysis targeted chimera (PROTAC). PROTACs are described in, for example, Published US Application Nos. 20210015942, 20210015929, 20200392131, 20200216507, US20200199247 and US20190175612; the disclosures of which are incorporated by reference herein.

### Ligands

In some embodiments, a Drug unit includes ligands that can be bound by a Carboxyl unit, such as platinum (Pt), ruthenium (Ru), rhodium (Rh), gold (Au), silver (Ag), copper (Cu), molybdenum (Mo), titanium (Ti), or iridum (Ir); a radioisotope such as yittrium-88, yittrium-90, technetium-99, copper-67, rhenium-188, rhenium-186, galium-66, galium-67, indium-111, indium-114, indium-115, lutetium-177, strontium-89, sararium-153, and lead-212.

### Drug-Linker Compounds

In some embodiments, provided are Drug-Linker compounds comprising a Linker compound as described above with at least one Drug unit attached via the attachment site.

In some embodiments, provided are Drug-Linker compounds comprising a Drug unit is selected from a cytotoxic agent, an immune modulatory agent, a nucleic acid, a growth inhibitory agent, a PROTAC, a toxin, a radioactive isotope and a chelating ligand. In more specific embodiments, the Drug unit is a cytotoxic agent. In more specific embodiments, the cytotoxic agent is selected from the group consisting of an auristatin, a maytansinoid, a camptothecin, a duocarmycin, and a calicheamicin. In some embodiments, the cytotoxic agent is an auristatin. In some embodiments, the cytotoxic agent is MMAE or MMAF. In some embodiments, the cytotoxic agent is SS-exatecan. In some embodiments, the cytotoxic agent is RS-exatecan. In some embodiments, the cytotoxic agent is a camptothecin. In some embodiments, the cytotoxic agent is exatecan or SN-38. In some embodiments, the cytotoxic agent is exatecan. In some embodiments, the cytotoxic agent is DxD. In some embodiments, the cytotoxic agent is a calicheamicin. In some embodiments, the cytotoxic agent is a maytansinoid. In more specific embodiments, the maytansinoid is maytansine, maytansinol or ansamatocin-2.

In some embodiments, provided are Drug-Linker compounds comprising a Linker compound as described above with a Drug unit attached via the attachment site, wherein the Drug unit is an immune modulatory agent. For example, in some embodiments the immune modulatory agent is selected from a TRL7 agonist, a TLR8 agonist, a STING agonist, or a RIG-1 agonist. In more specific embodiments, the immune modulatory agent is an TLR7 agonist. In more specific embodiments, the TLR7 agonist is an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, a benzonaphthyridine, a guanosine analog, an adenosine analog, a thymidine homopolymer, ssRNA, CpG-A, PolyG10, or PolyG3. In certain embodiments, the immune modulatory agent is a TLR8 agonist. In more specific embodiments, the TLR8 agonist is selected from an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine or a ssRNA. In some embodiments, the immune modulatory agent is a STING agonist. In other embodiments, the immune modulatory agent is a RIG-1 agonist. For example, in some embodiments, the RIG-1 agonist is selected from KIN1148, SB-9200, KIN700, KIN600, KIN500, KIN100, KIN101, KIN400 and KIN2000.

In some embodiments, provided are Drug-Linker compounds comprising a Linker compound as described above with a Drug unit attached via the attachment site, wherein the Drug unit is a chelating ligand. For example, in some embodiments the chelating ligand is selected from platinum (Pt), ruthenium (Ru), rhodium (Rh), gold (Au), silver (Ag), copper (Cu), molybdenum (Mo), titanium (Ti), or iridium (Ir); a radioisotope such as yittrium-88, yittrium-90, technetium-99, copper-67, rhenium-188, rhenium-186, galium-66, galium-67, indium-111, indium-114, indium-115, lutetium-177, strontium-89, sararium-153, and lead-212.

In some embodiments, provided is a Drug-Linker compound, represented by the structure of Formula (A)

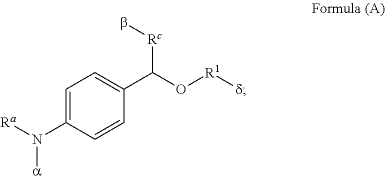

or a salt thereof, wherein:
(i) β is R²⁰(—R²¹—[O—CH₂—CH₂]ₙ₂₀—R²²—NR²⁴R²⁵)_{z}; wherein
   R²⁰ is NH,

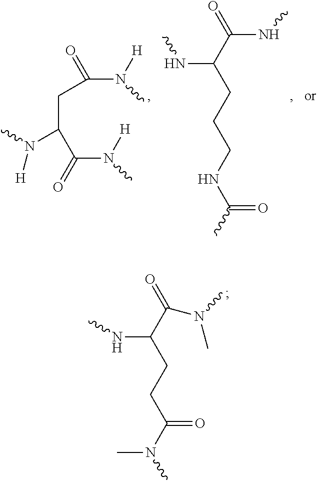

R²¹ and R²² are each, independently, a bond or C₁-C₃ alkylene;
R²⁴ and R²⁵ are each independently selected from H; polyhydroxyl group; and —C(O)— polyhydroxyl group; provided that R²⁴ and R²⁵ are not both H; z is 1, or 2; and
n20 is 2 to 26;
(ii) R^{a} is H or C₁-C₆ alkyl;
(iii) R^{c} is a bond, —C(O)—, —S(O)—, —SO₂—, C₁₋₆ alkylene, C₁₋₆ alkynylene, or C₁₋₆ alkynylene-triazolyl;
(iv) R¹ is a bond, —C(O)—, or C₁₋₆ alkylene; and
(v) δ is selected from a Drug;
(vi) α is represented by

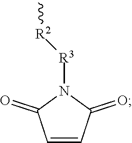

wherein
R² is a peptide having 2-5 amino acids;
R³ is —C₁-C₁₀ alkylene-C(═O)—, —C₁-C₁₀ alkylene-, —C₁-C₁₀ alkylene-C(O)NH—C₁-C₈alkylene-[O—CH₂—CH₂]ₙ—C(O)— (where n is 1 to 26), or —C₁-C₈ alkylene-(CH₂—O—CH₂)_{b}—C(═O)— (where b is 1 to 26).

In some embodiments, for a Drug-Linker, R²⁰ is

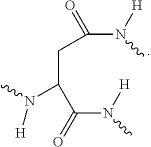

In some embodiments, for a Drug-Linker, z is 1.

In some embodiments, for a Drug-Linker, z is 2.

In some embodiments, for a Drug-Linker, R²⁴ and R²⁵ are each independently selected from a polyhydroxyl group.

In some embodiments, for a Drug-Linker, R²¹ is C₁-C₃ alkylene. In some cases, R²¹ is C₂ alkylene.

In some embodiments, for a Drug-Linker, R²² is a bond.

In some embodiments, for a Drug-Linker, β is

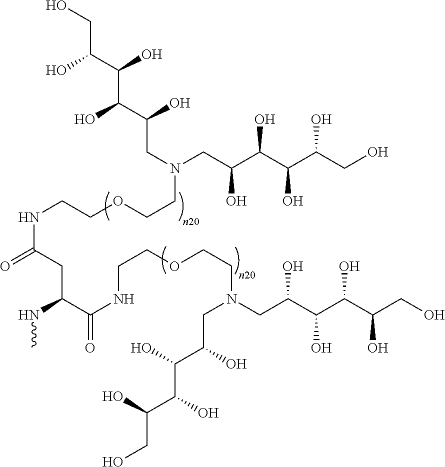

and n20 is 4 to 12;

In some embodiments, for a Drug-Linker, wherein s is

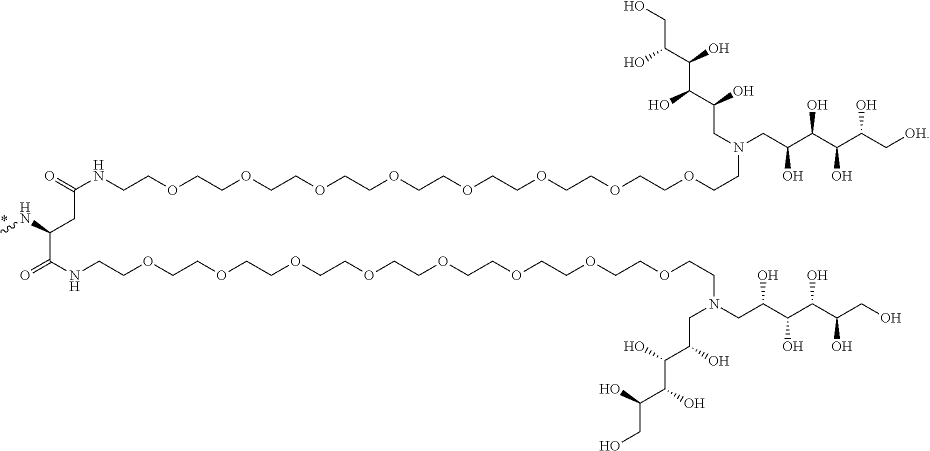

In some embodiments, for a Drug-Linker, R² is selected from:

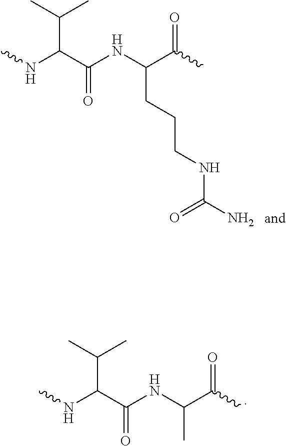

In some embodiments, for a Drug-Linker, R² is selected from:

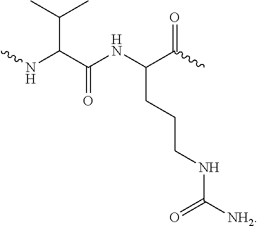

In some embodiments, for a Drug-Linker R³ is selected from:

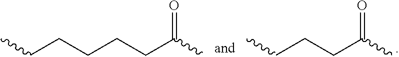

In some embodiments, for a Drug-Linker, R³ is selected from:

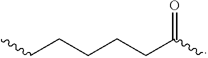

In some embodiments, for a Drug-Linker, R^{c} is a —C(O)— or C₁₋₆ alkynylene-triazolyl;

In some embodiments, for a Drug-Linker, R^{c} is —C(O)—.

In some embodiments, for a Drug-Linker, R¹ is —C(O)—.

In some embodiments, for a Drug-Linker, the Drug is selected from a cytotoxic agent. In some embodiments, the Drug is MMAE or MMAF. In some embodiments, the Drug is MMAE. In some embodiments, the Drug is exatecan.

In some embodiments, for a Drug-Linker, the Drug is selected from an immune modulatory agent.

In some embodiments, for a Drug-Linker, δ is selected from

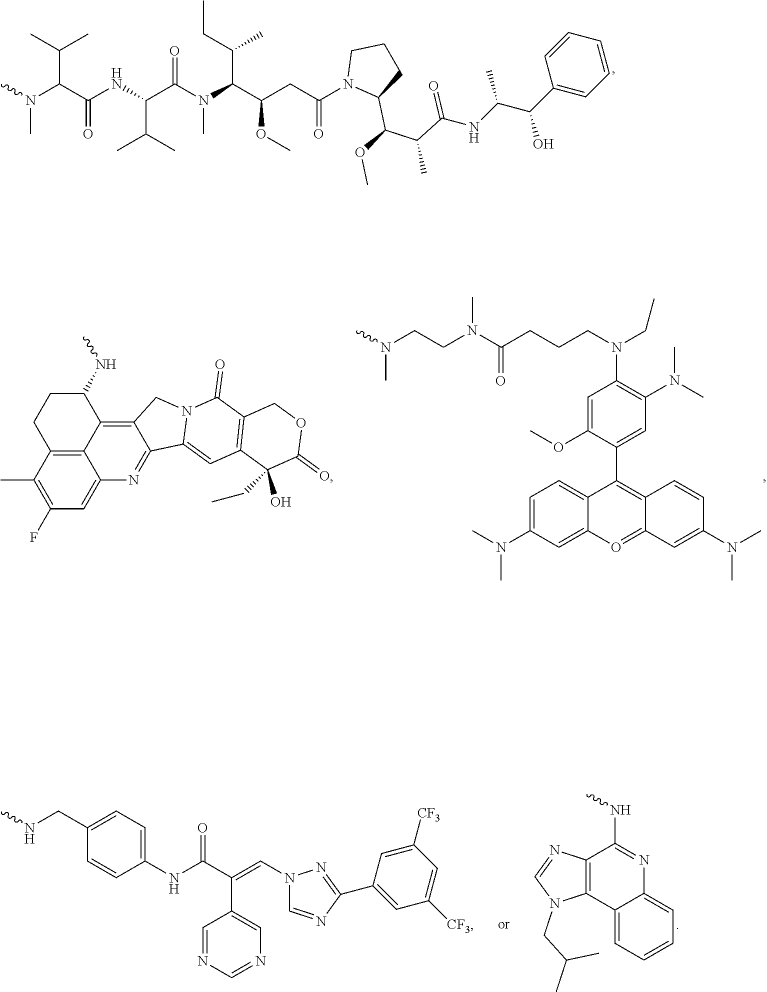

In some embodiments, δ is

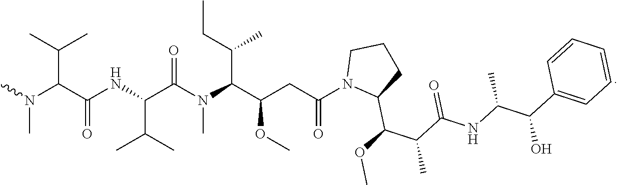

In some embodiments, δ is

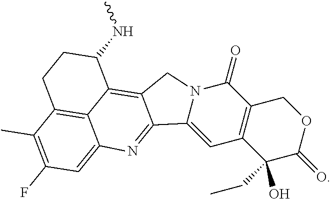

In some embodiments, the wavy bond indicates a point of attachment to the Drug-Linker.

In some embodiments, provided are Drug-Linker compounds selected from following

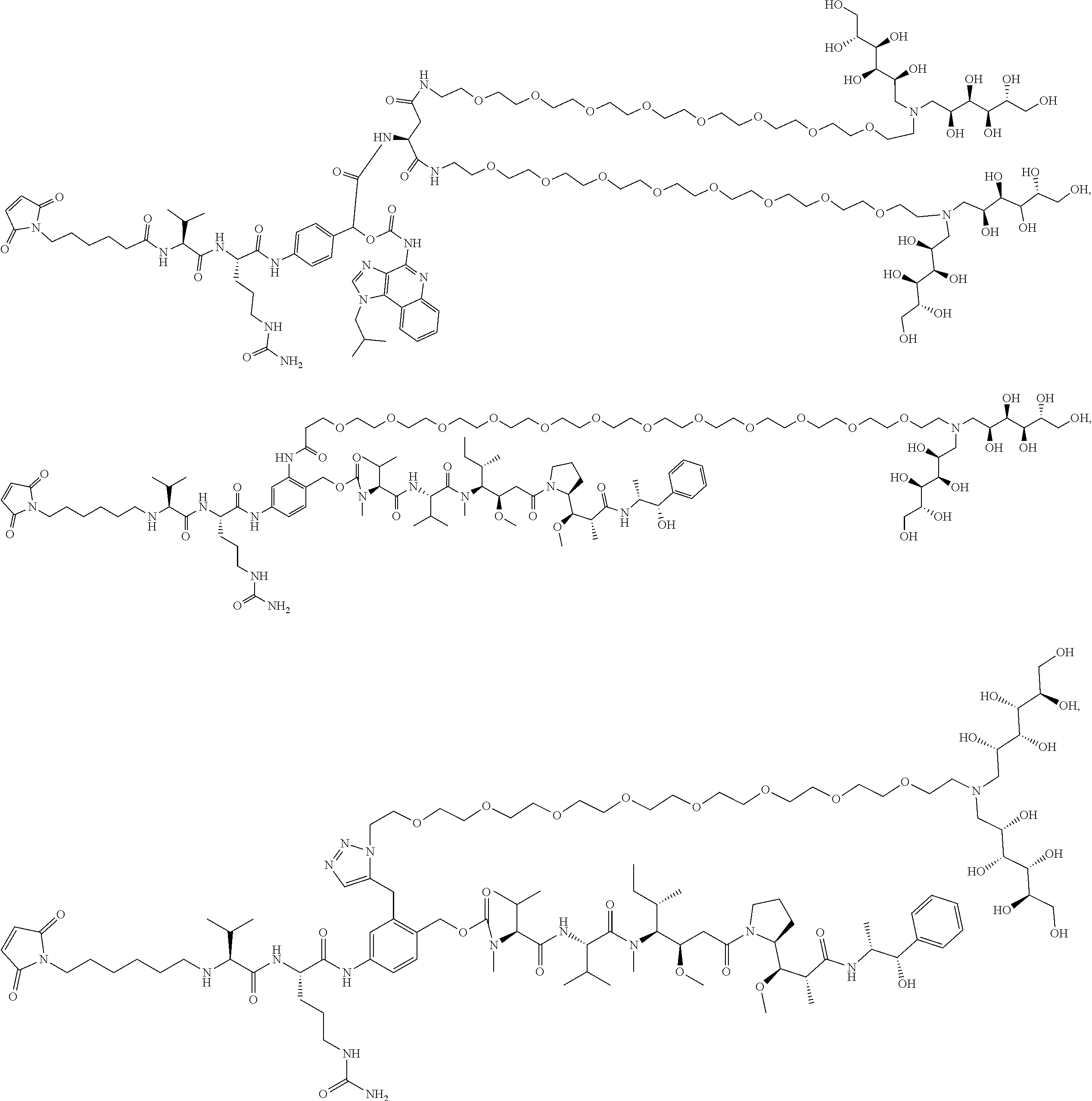

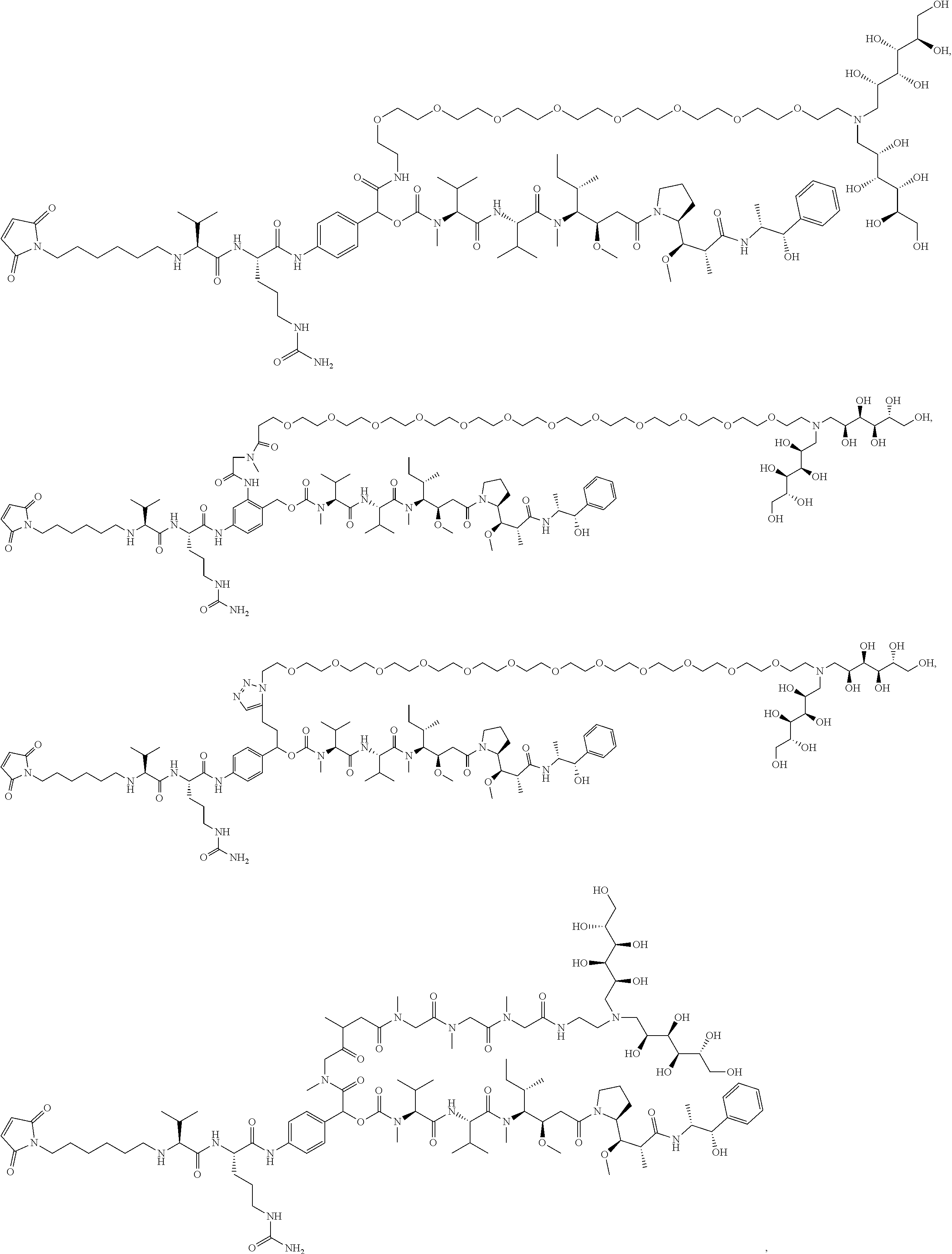

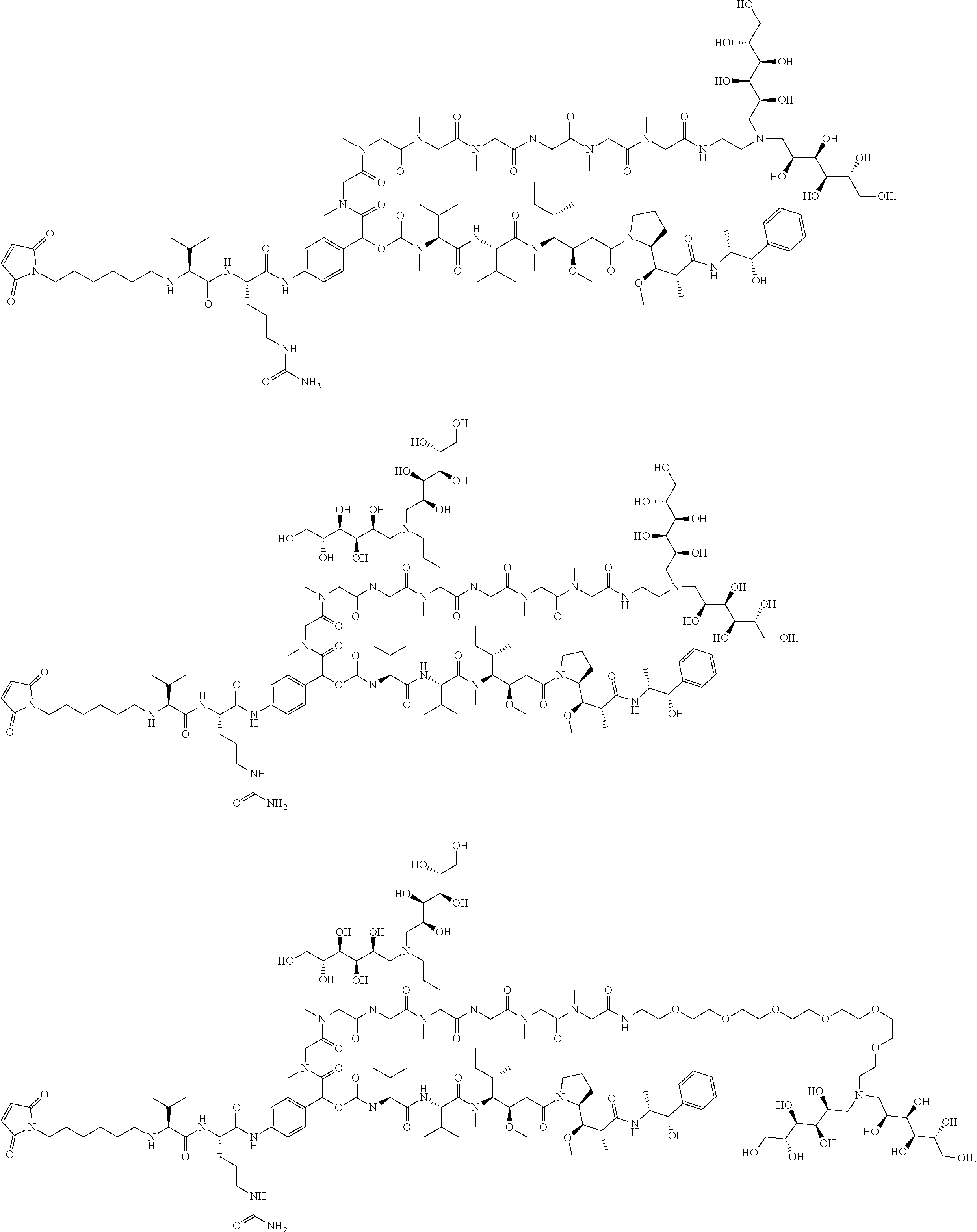

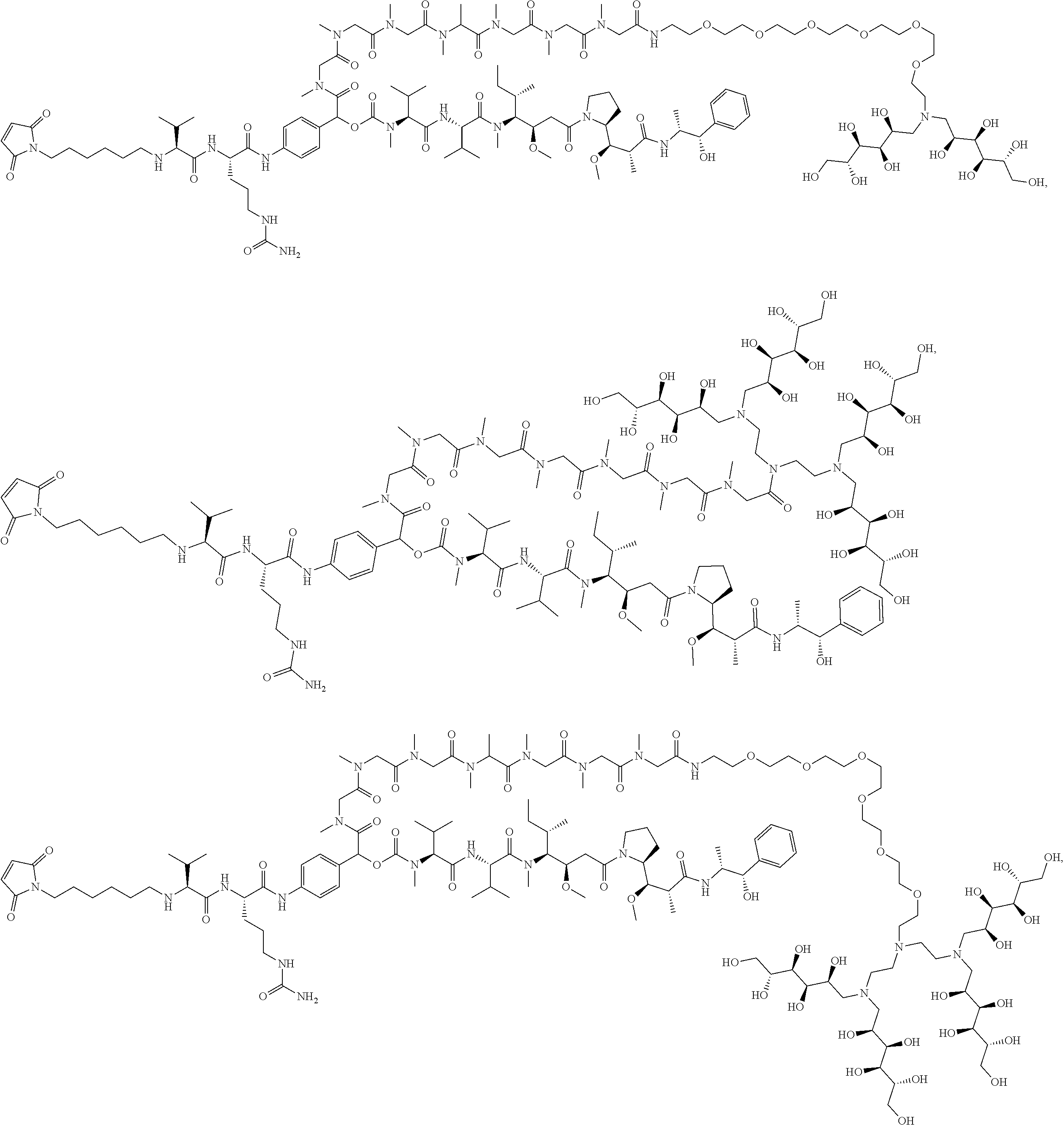

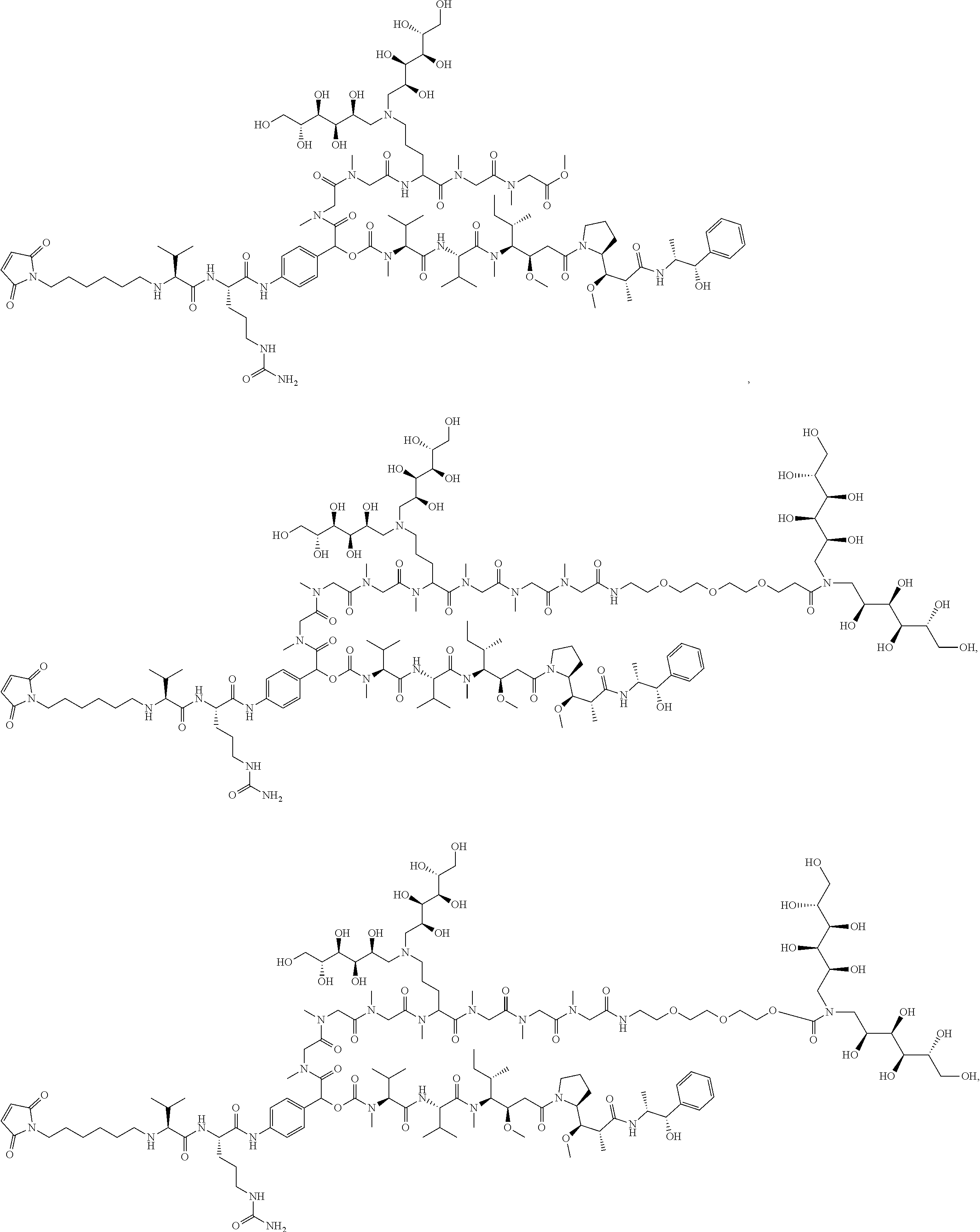

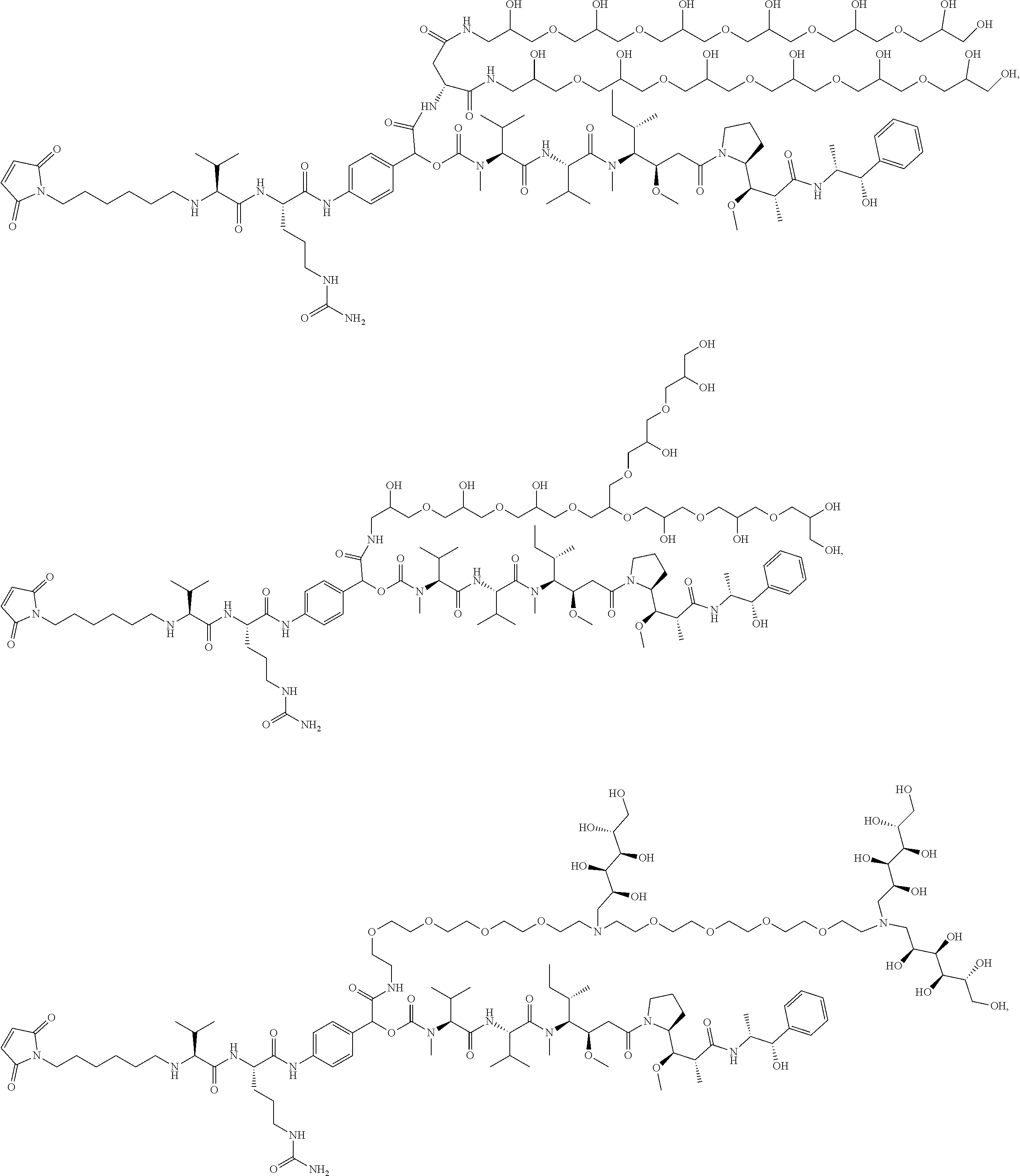

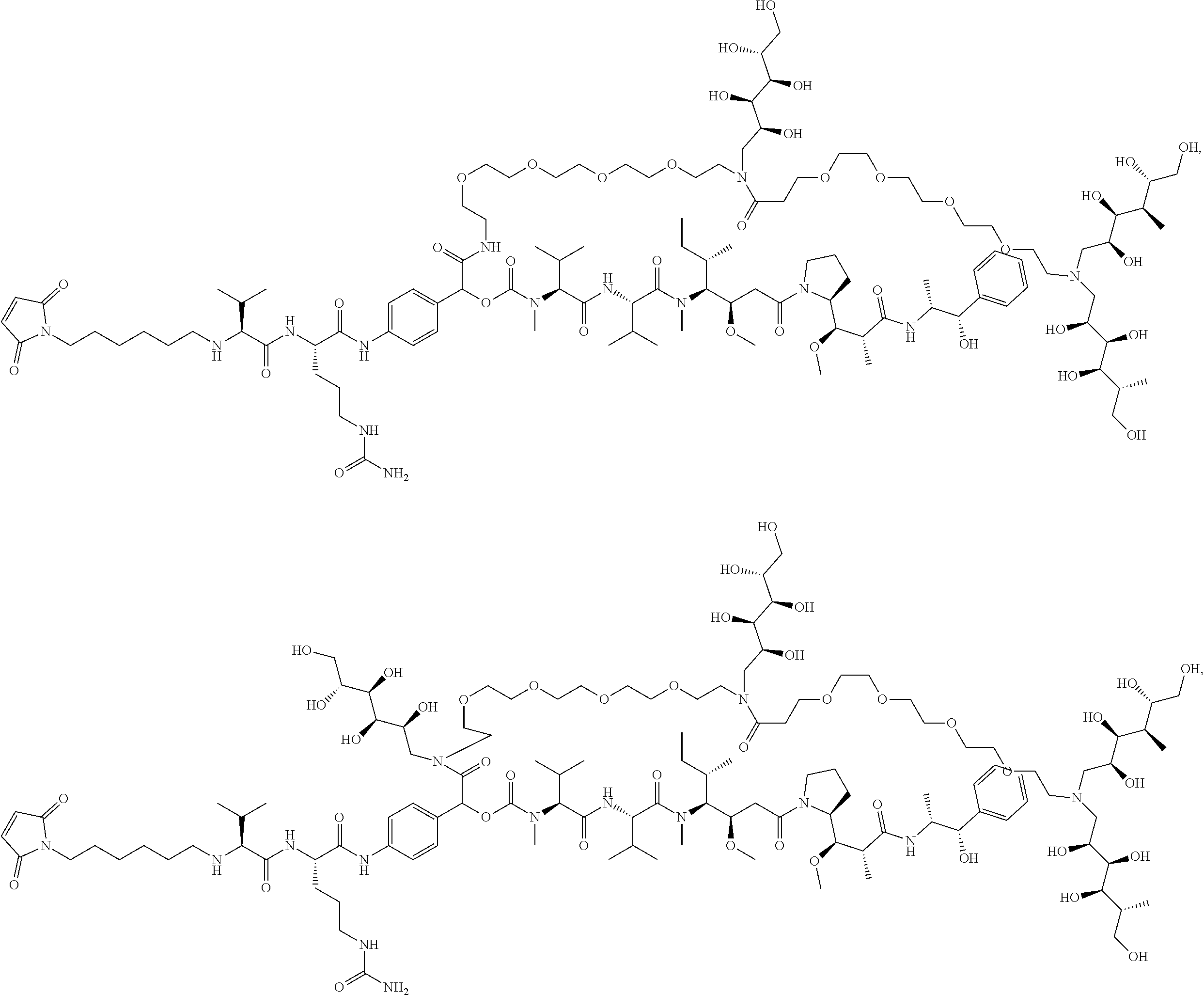

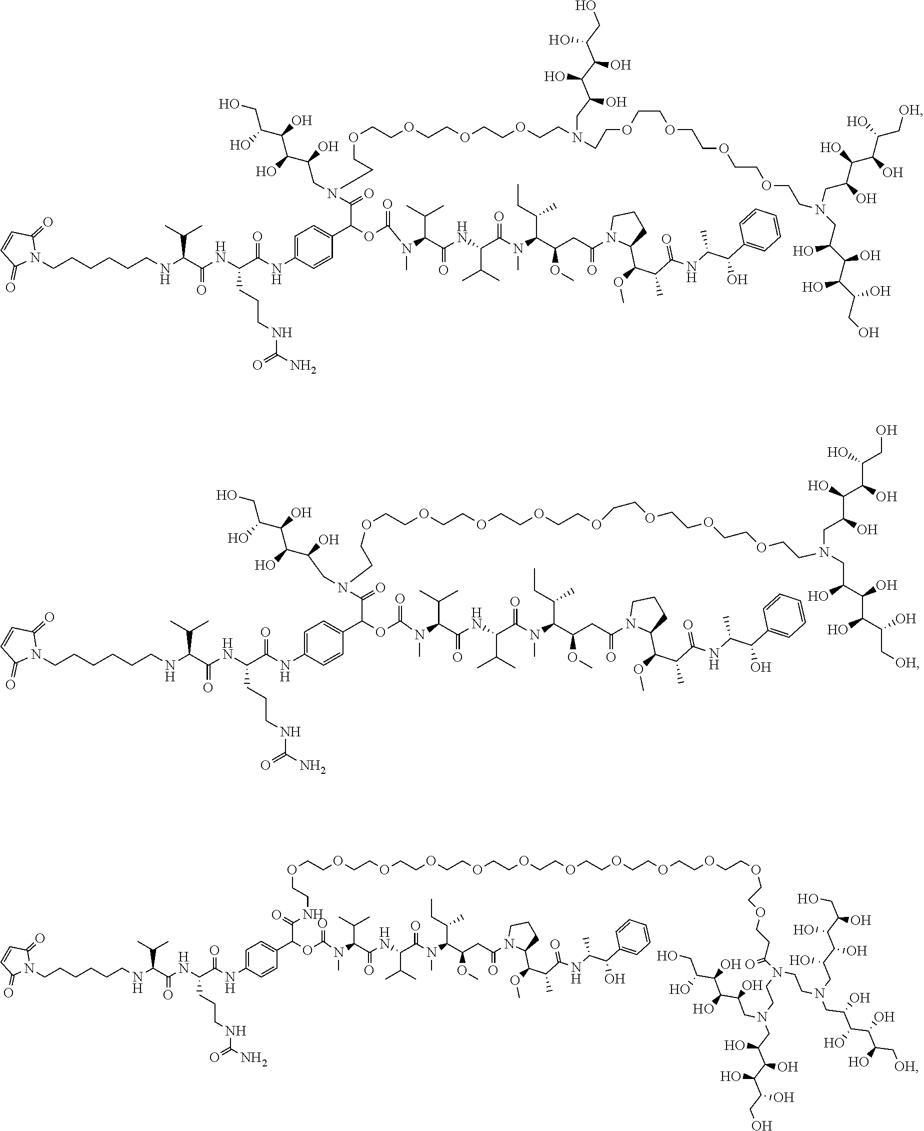

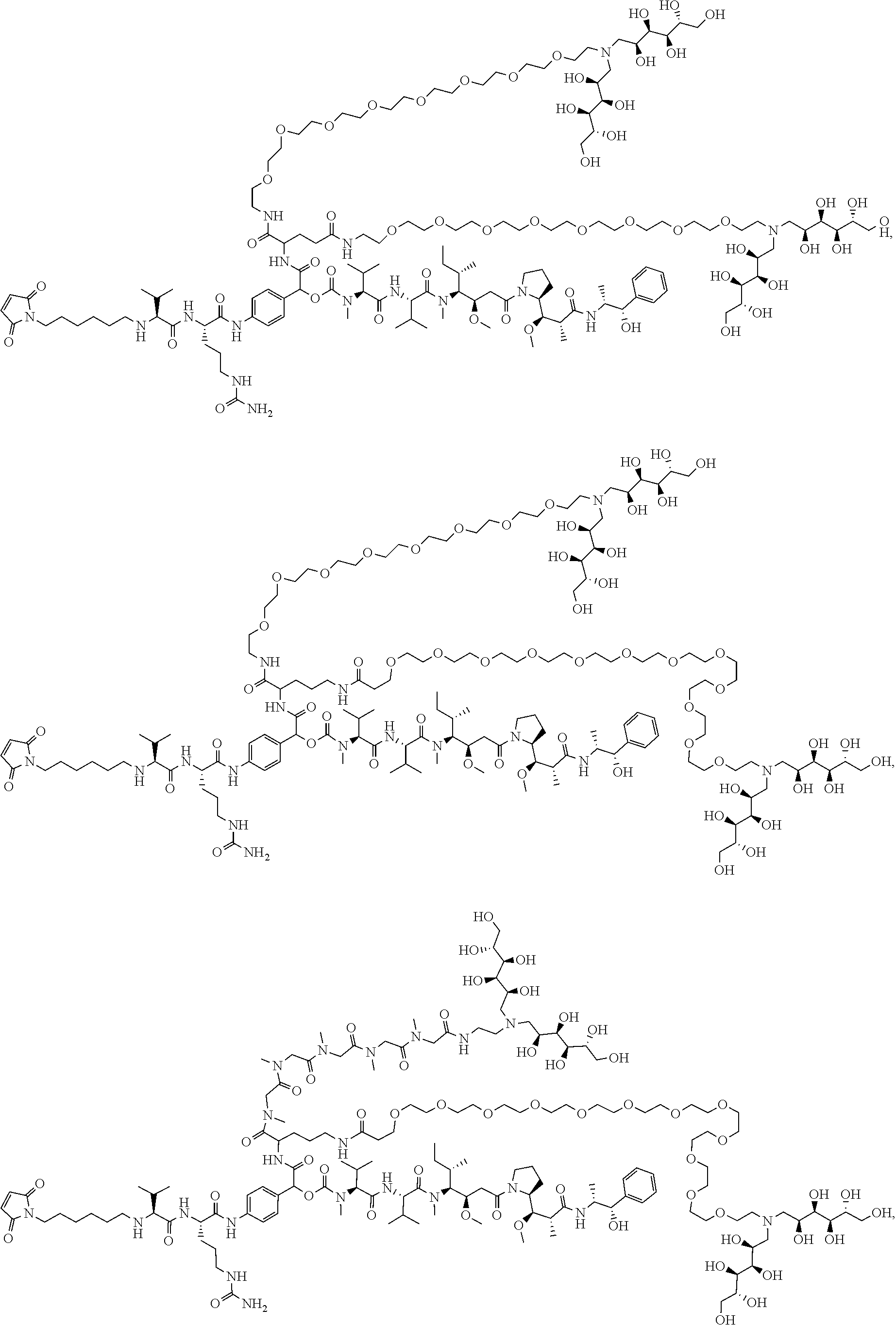

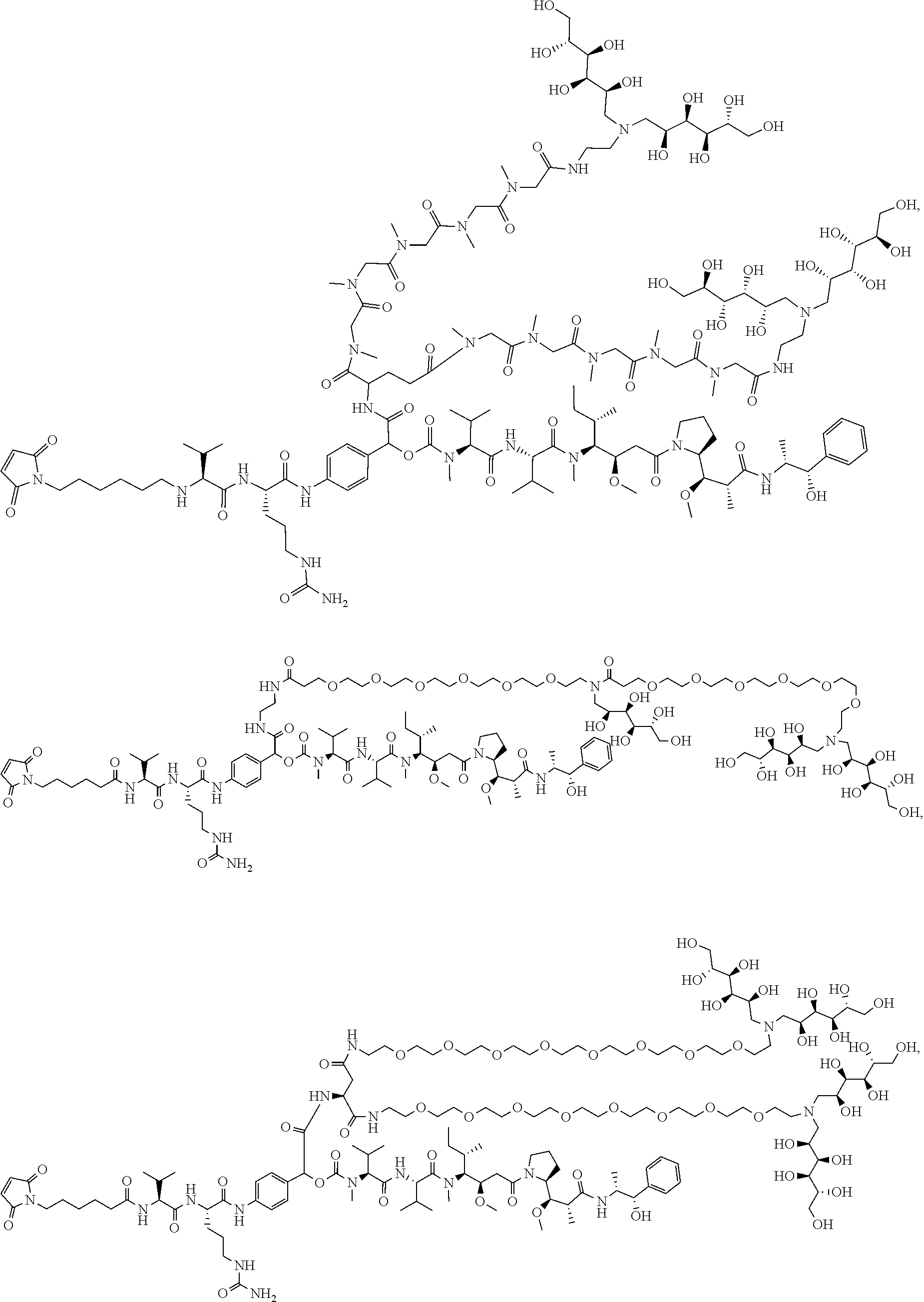

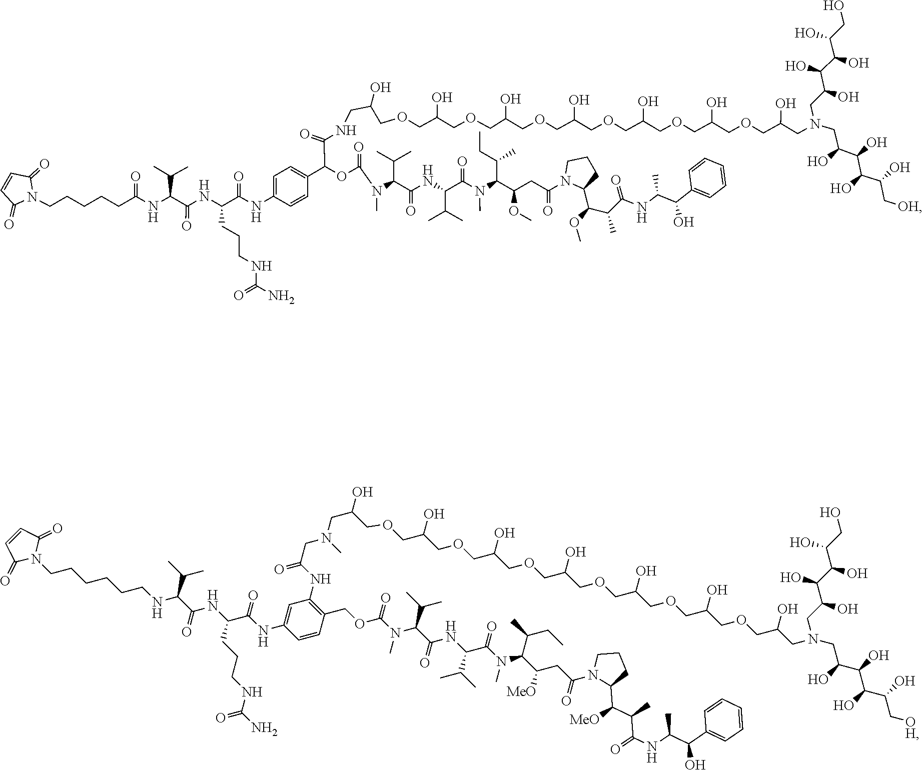

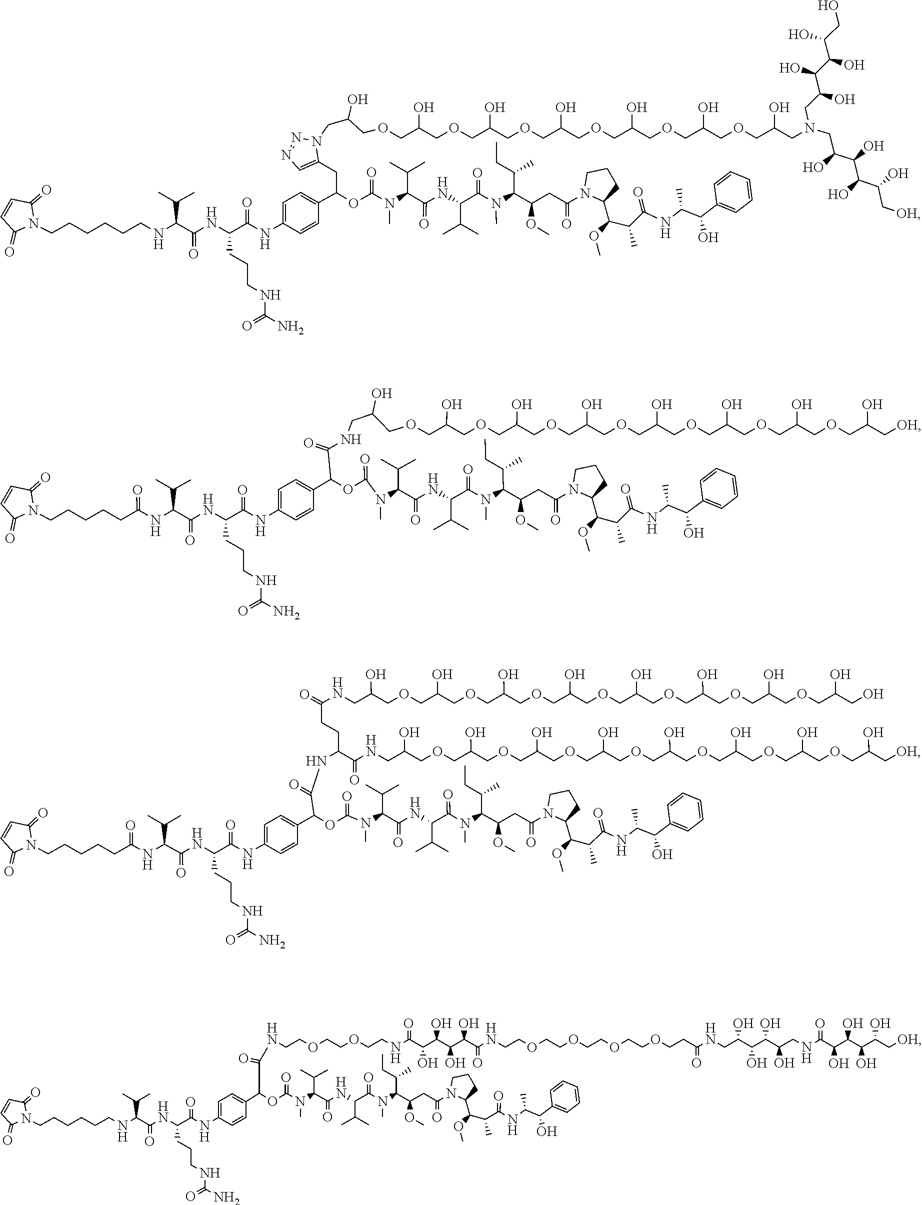

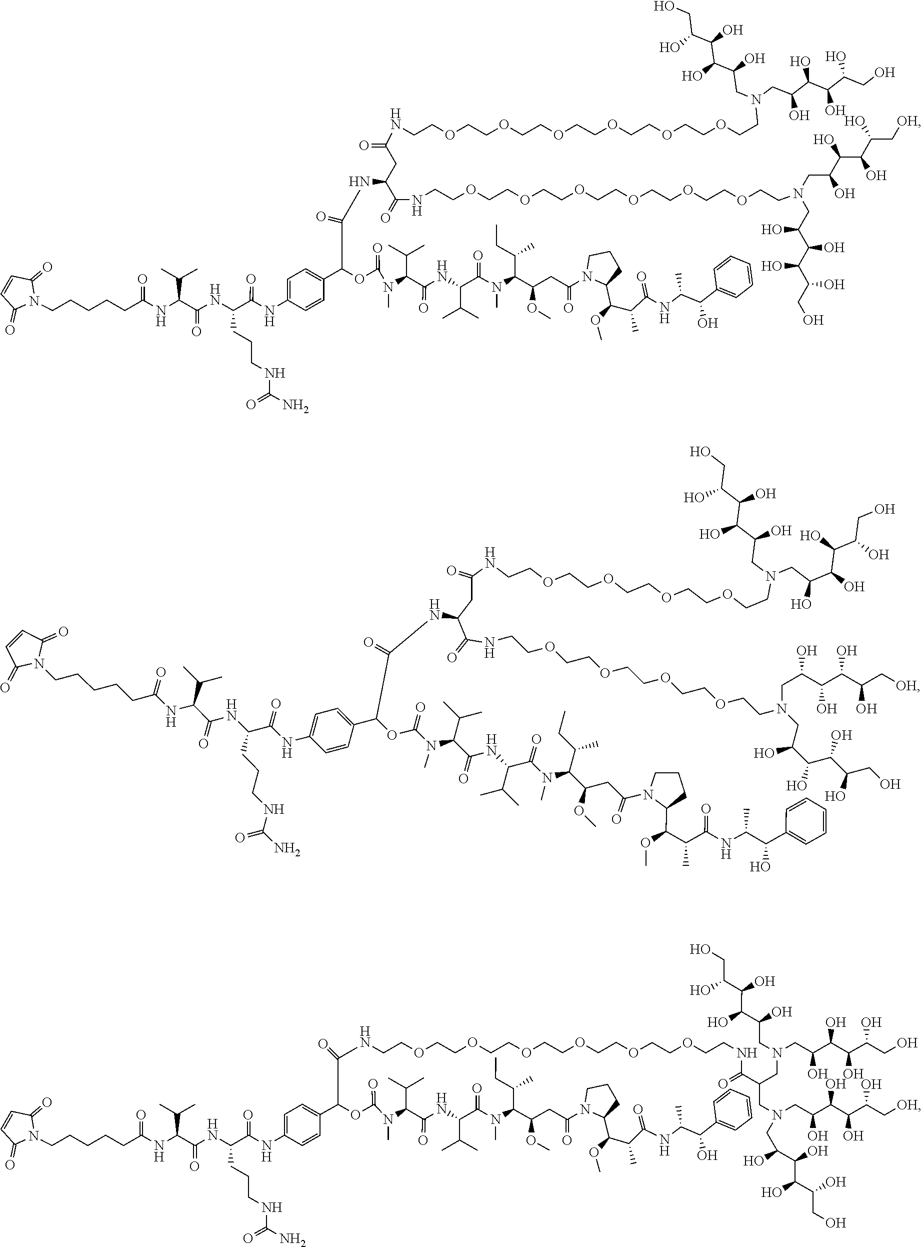

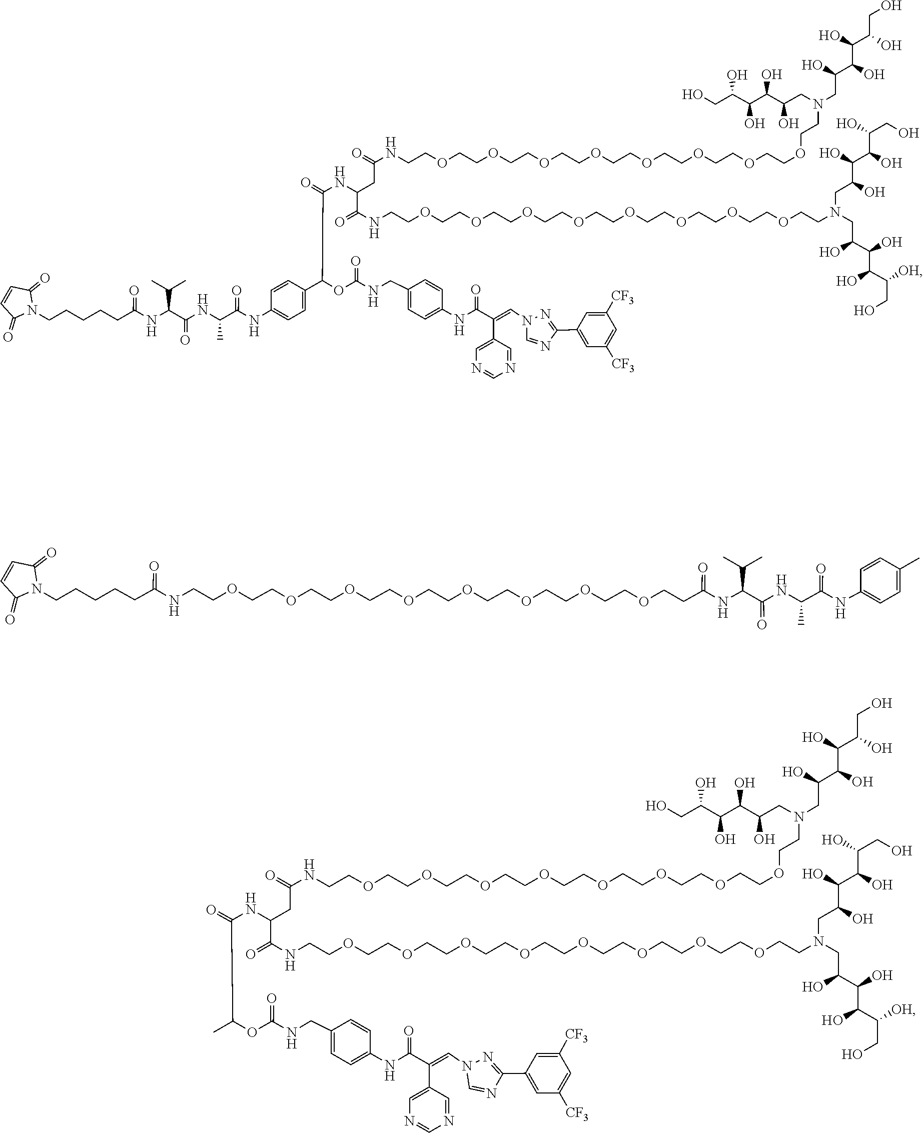

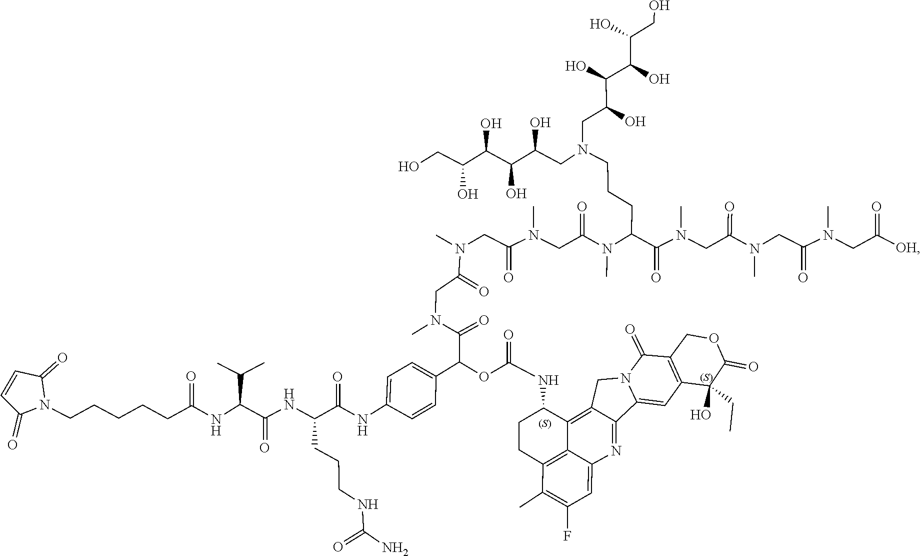

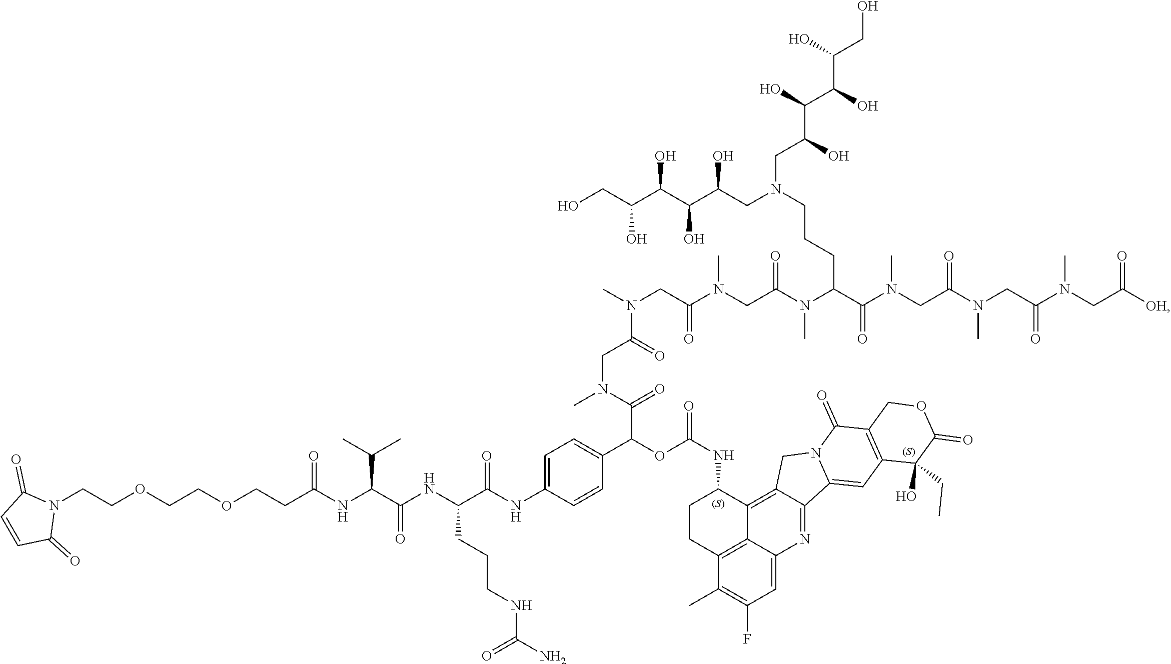

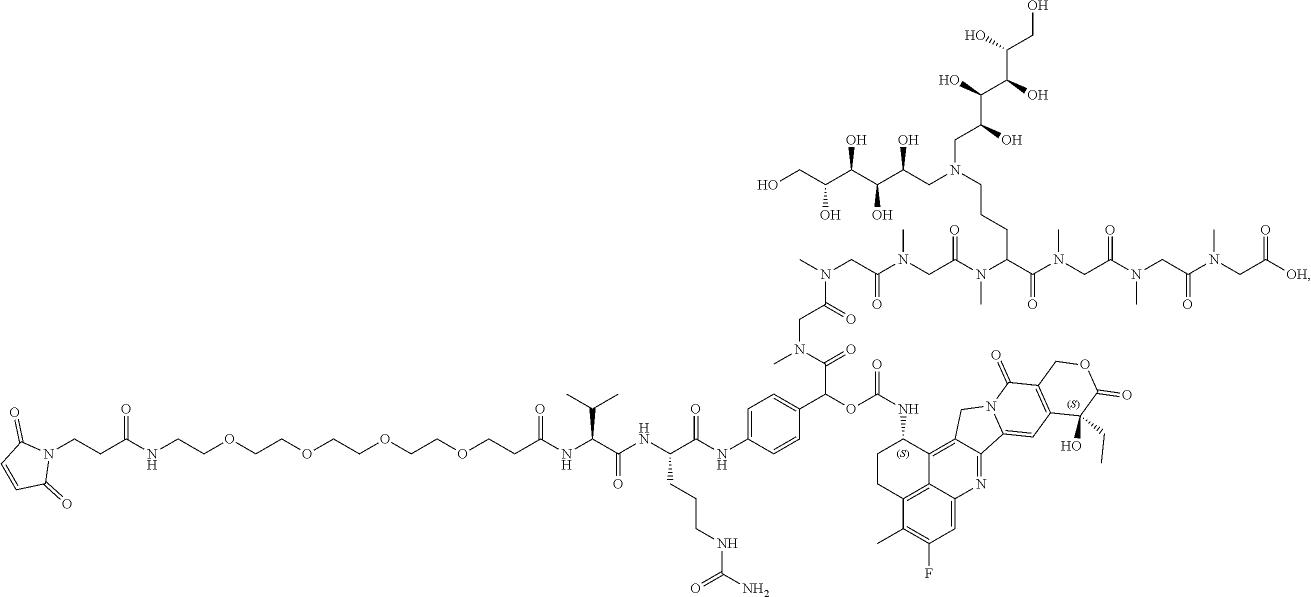

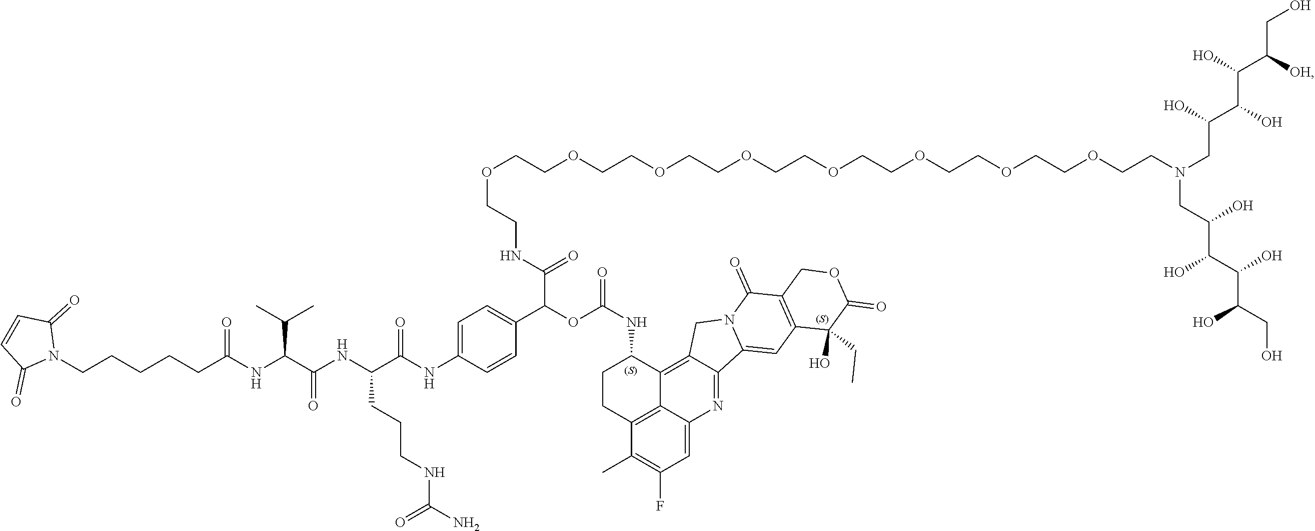

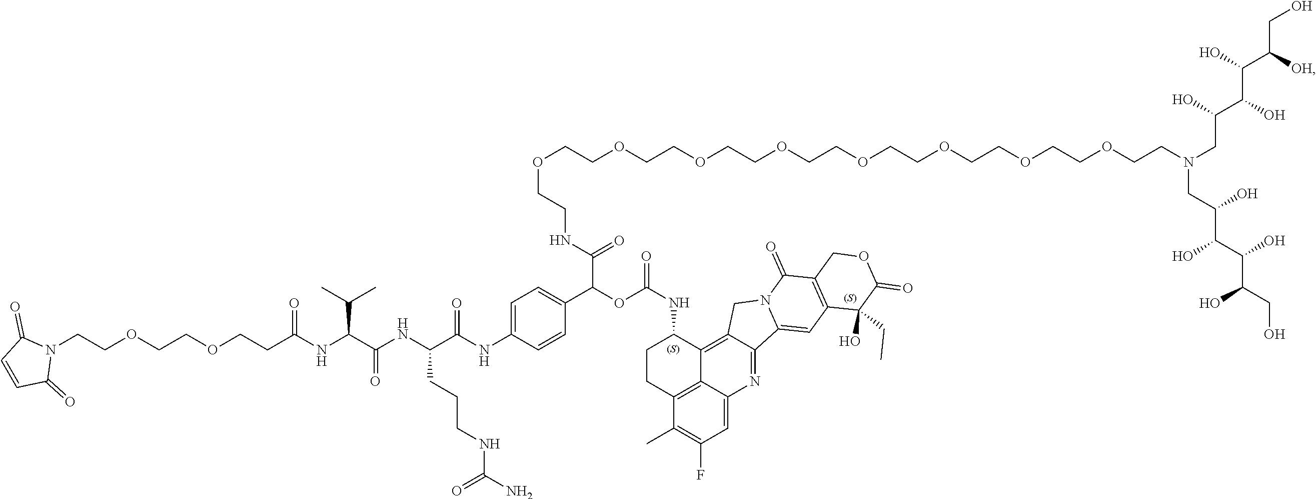

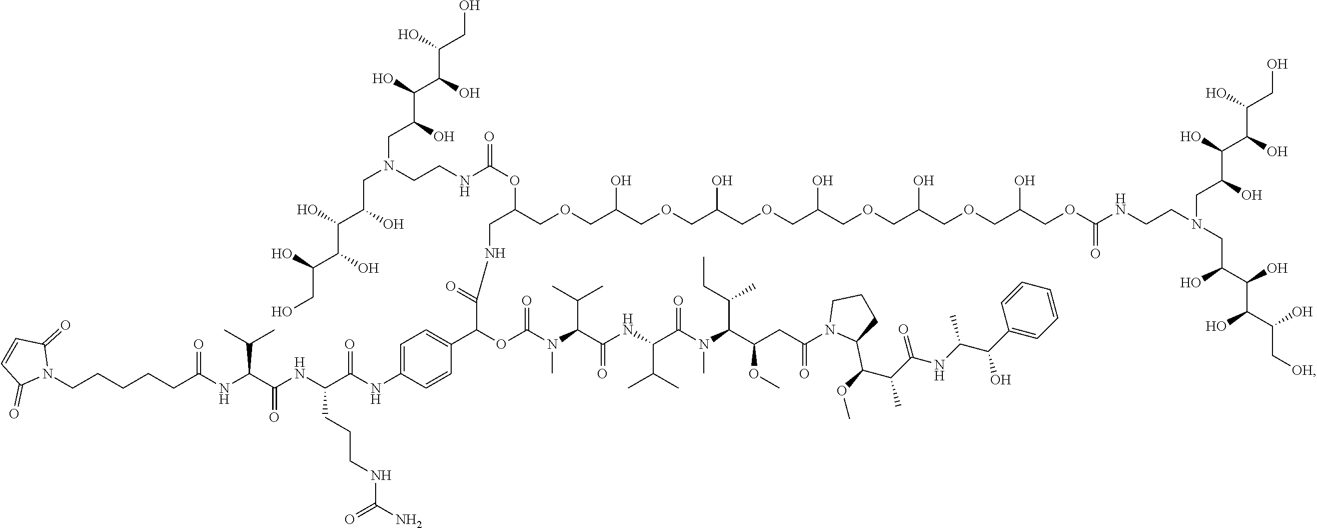 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCC(COCC(O)COCC(O)COCC(O)COCC(O)COCC(O)COC(=O)NCCN(C[C@H](O)[C@@H](O)[C@H](O)[C@H](O)CO)C[C@H](O)[C@@H](O)[C@H](O)[C@H](O)CO)OC(=O)NCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

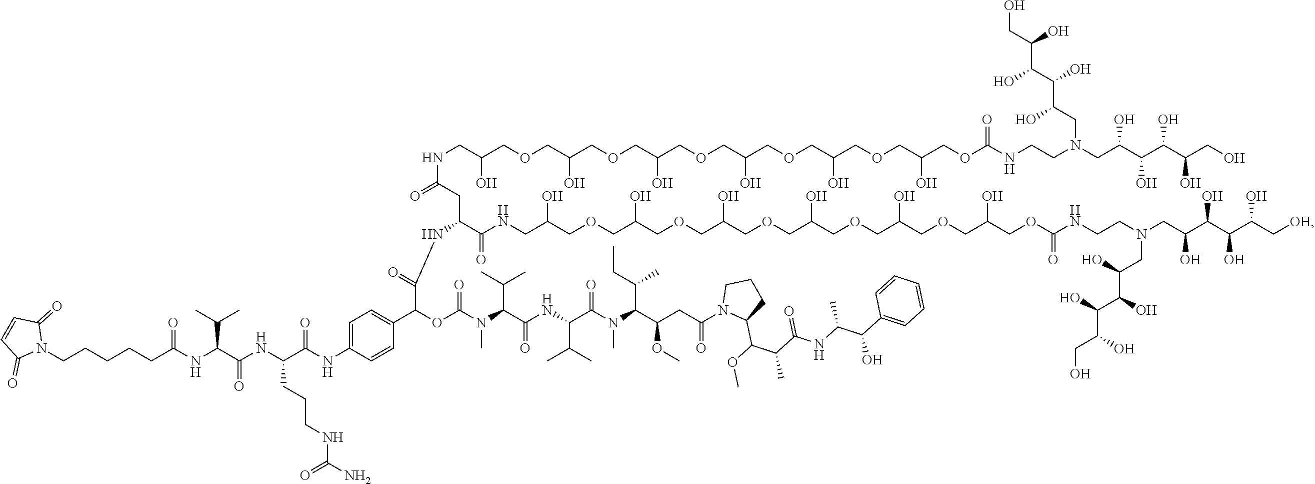 [SMILES: CC[C@H](C)[C@@H]([C@@H](CC(=O)N1CCC[C@H]1C(OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@H](CC(=O)NCC(O)COCC(O)COCC(O)COCC(O)COCC(O)COCC(O)COC(=O)NCCN(C[C@H](O)[C@@H](O)[C@H](O)[C@H](O)CO)C[C@H](O)[C@@H](O)[C@H](O)[C@H](O)CO)C(=O)NCC(O)COCC(O)COCC(O)COCC(O)COCC(O)COCC(O)COC(=O)NCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

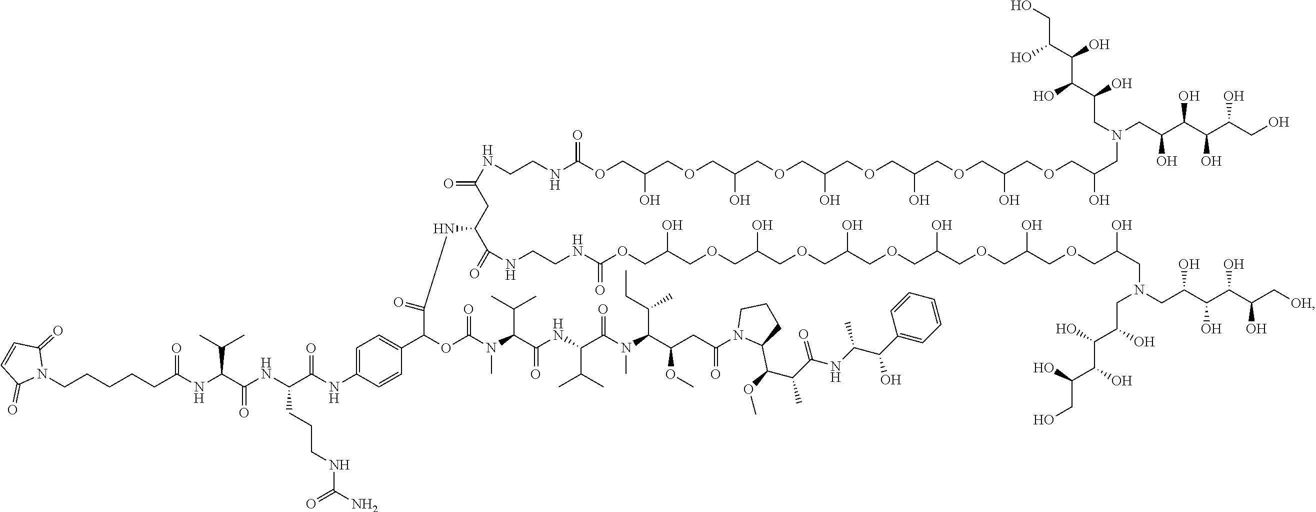 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@H](CC(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

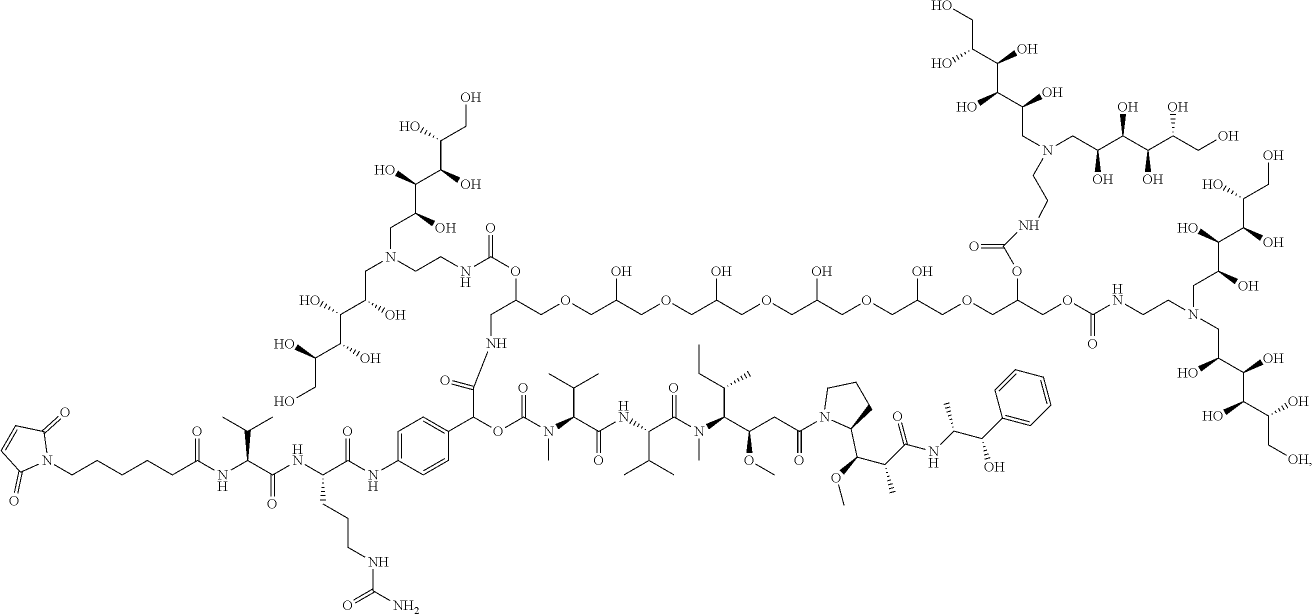

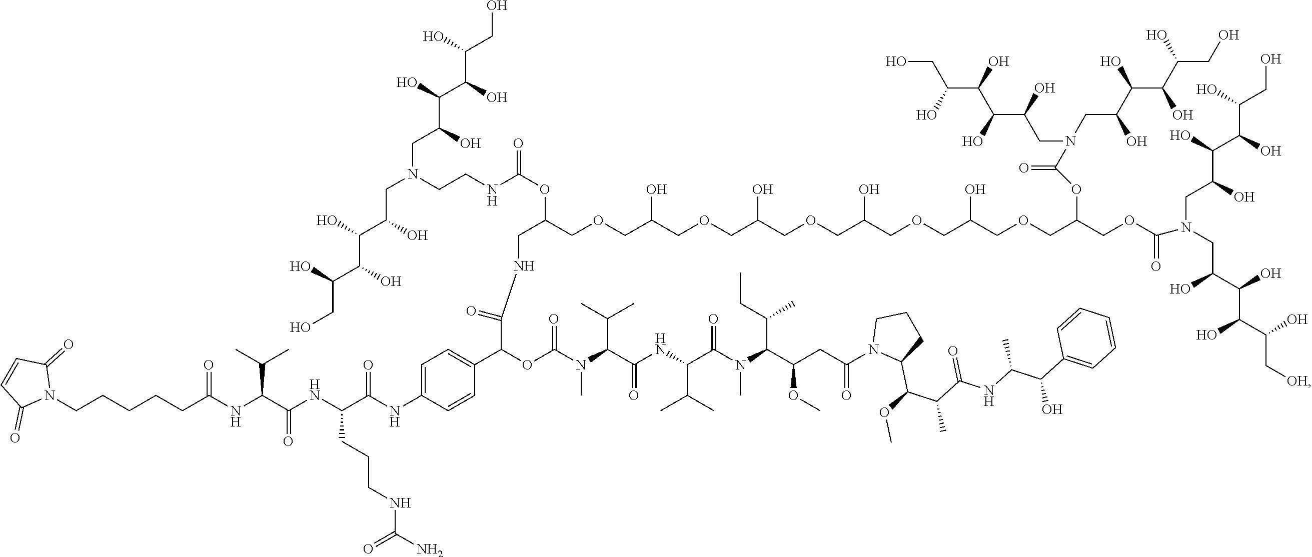

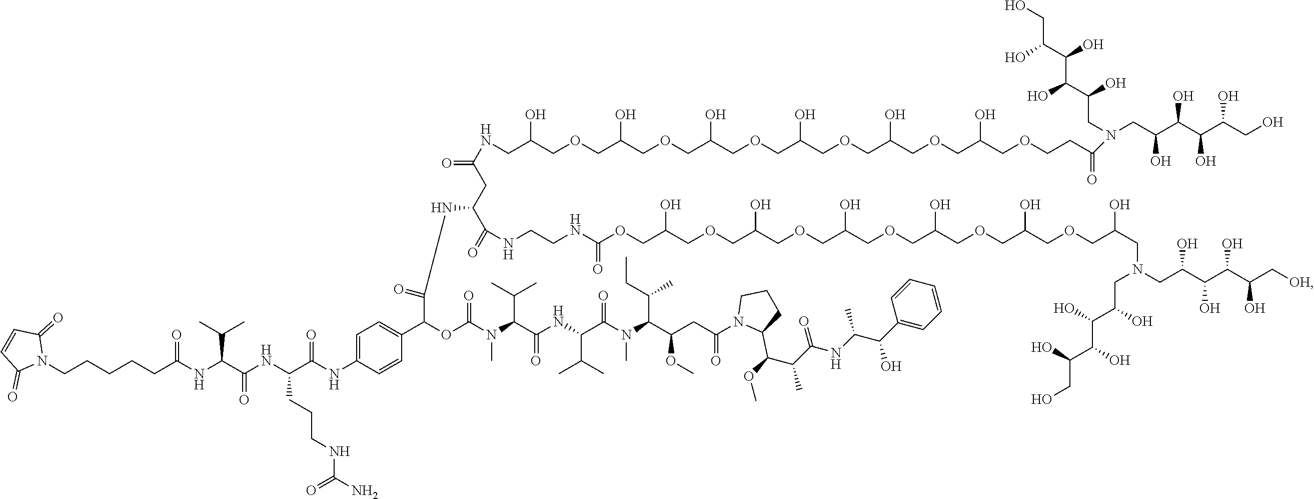 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@H](CC(=O)NCC(O)COCC(O)COCC(O)COCC(O)COCC(O)COCC(O)COCCC(=O)N(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

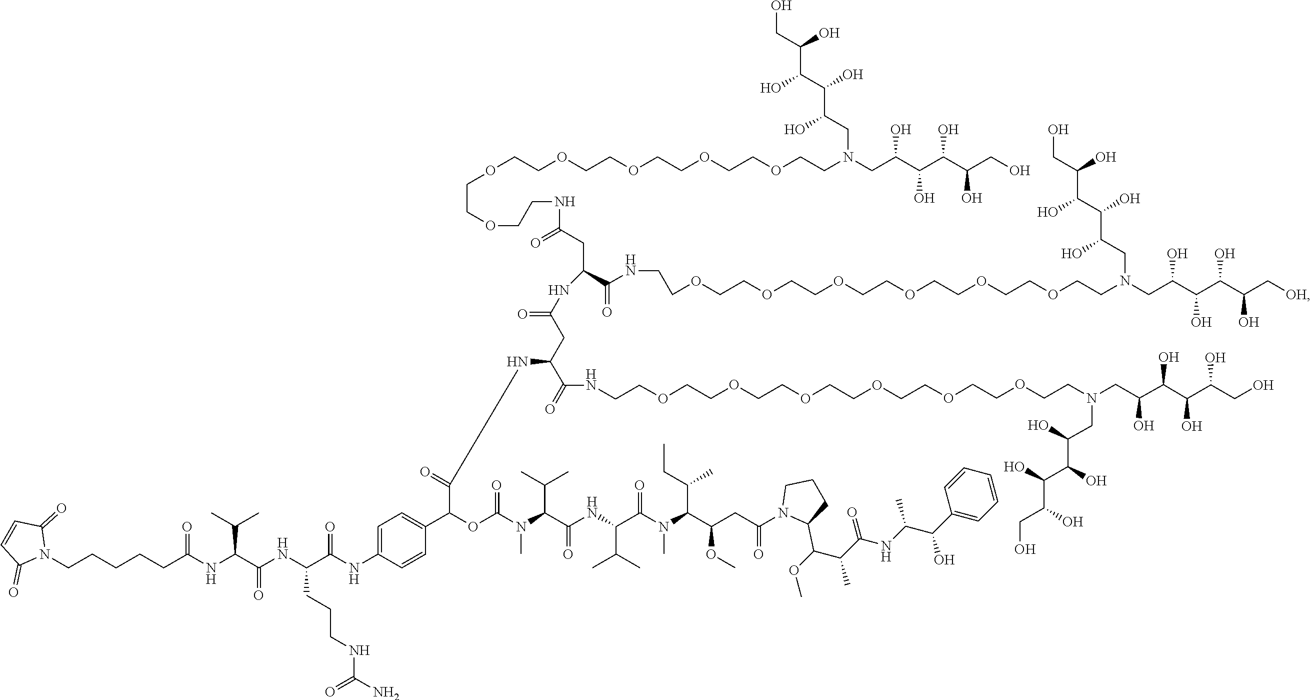

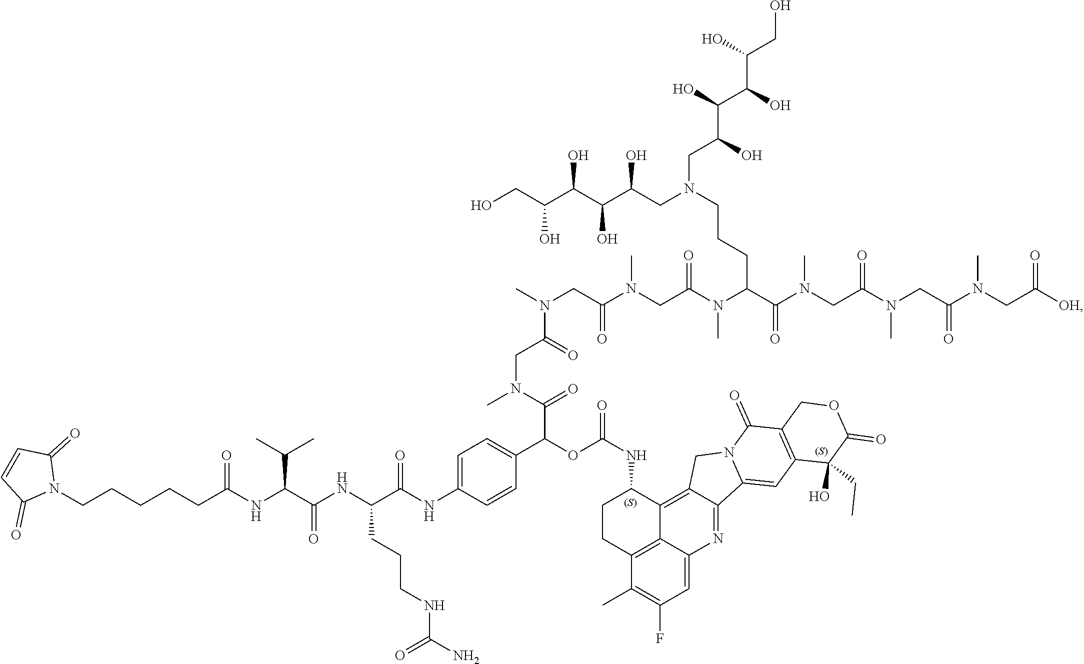

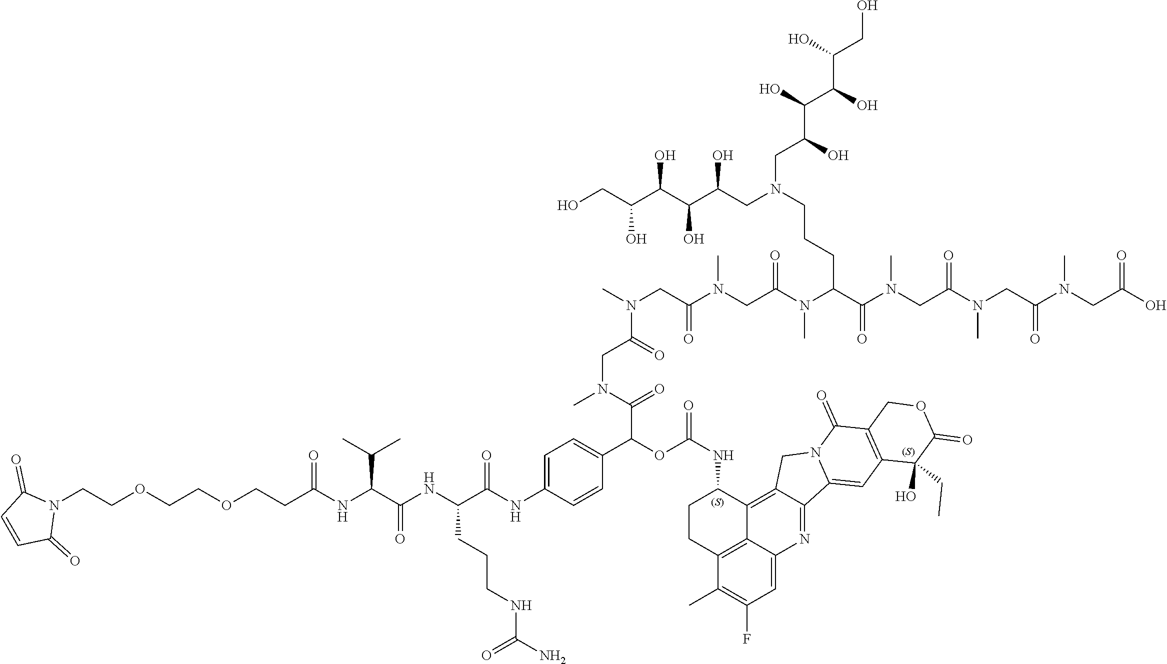

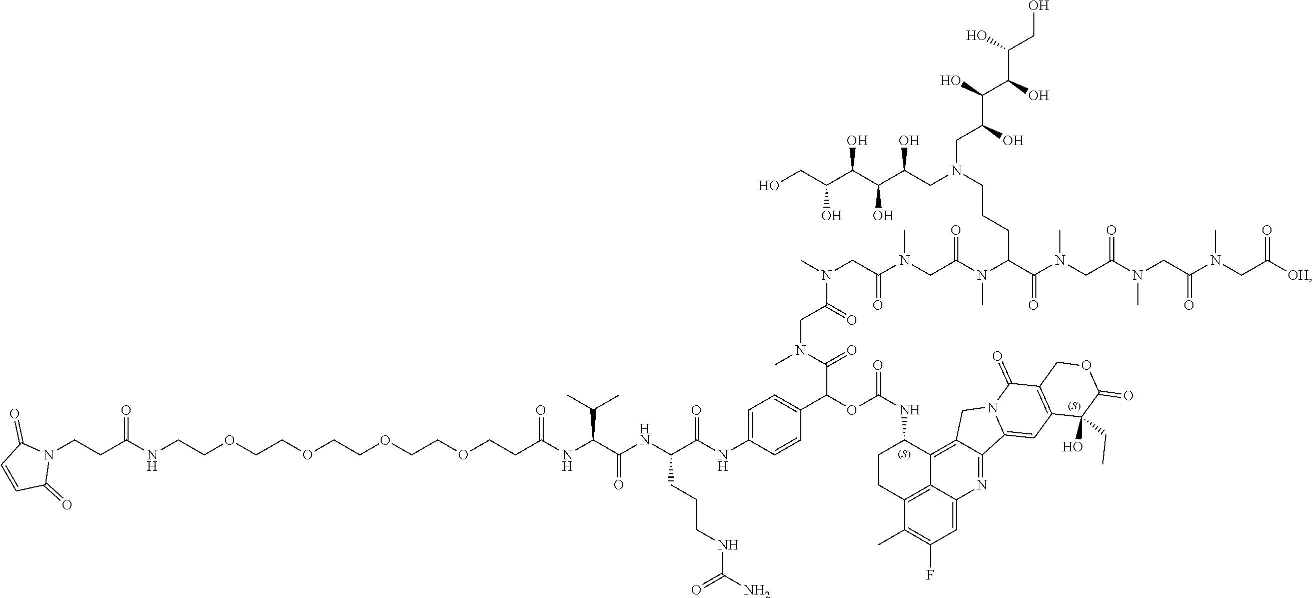

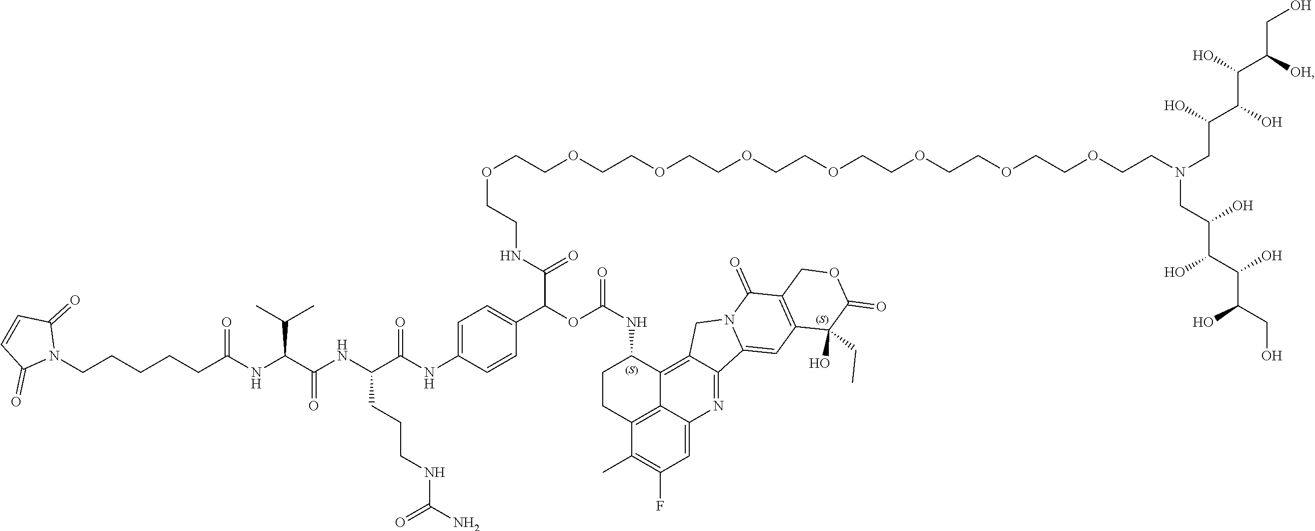

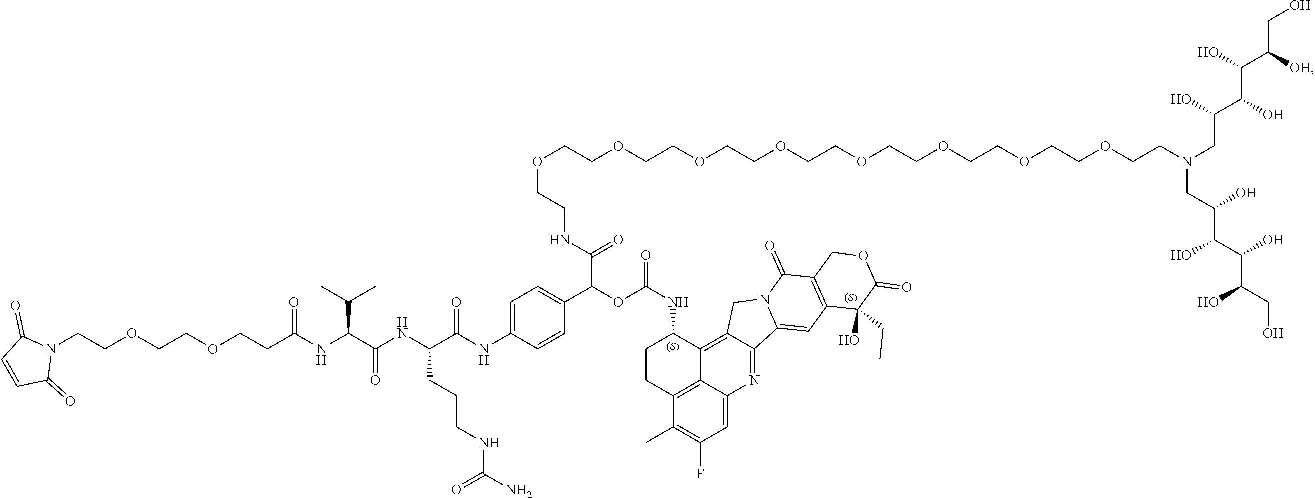

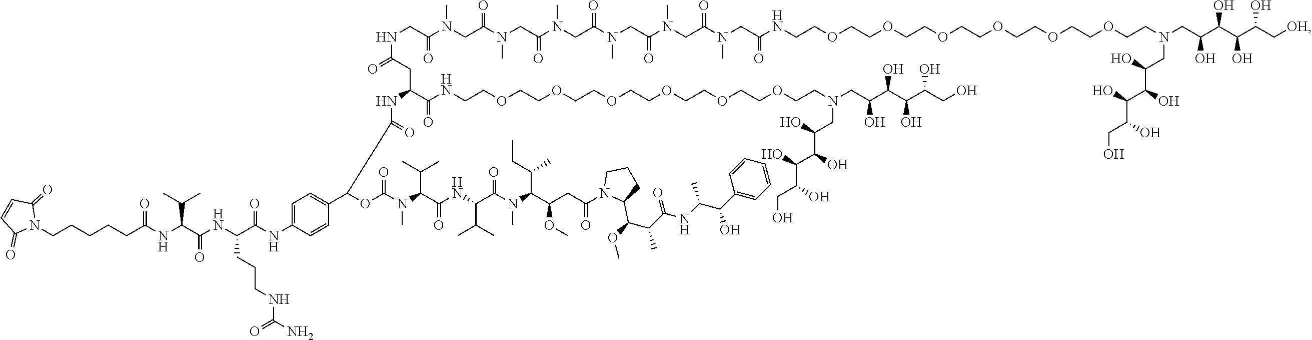

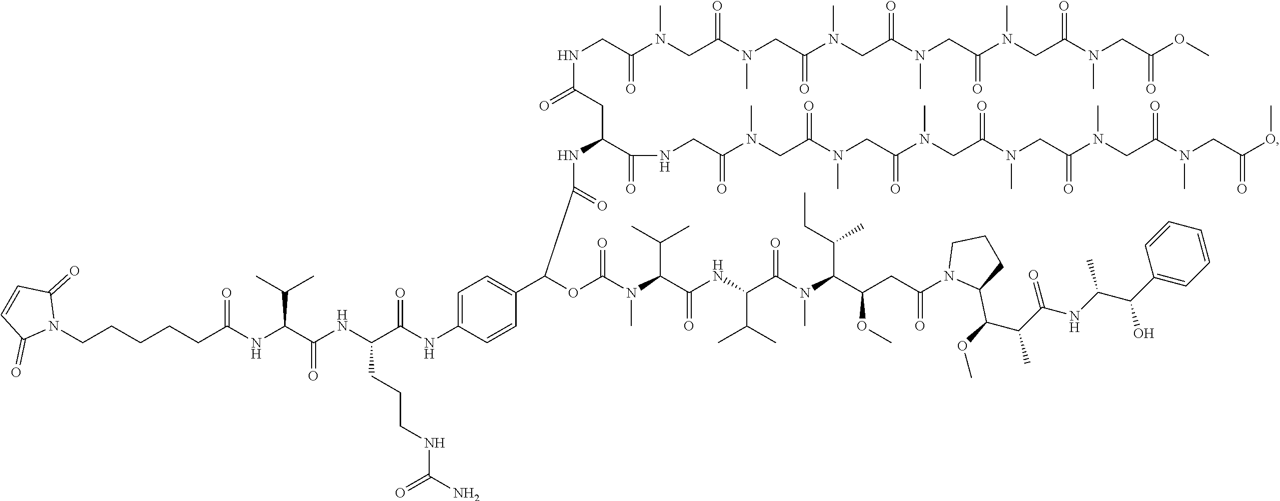 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@@H](CC(=O)NCC(=O)N(C)CC(=O)N(C)CC(=O)N(C)CC(=O)N(C)CC(=O)N(C)CC(=O)N(C)CC(=O)OC)C(=O)NCC(=O)N(C)CC(=O)N(C)CC(=O)N(C)CC(=O)N(C)CC(=O)N(C)CC(=O)N(C)CC(=O)OC)c1ccc(NC(=O)[C@H](CCCNC(N)=O)NC(=O)[C@@H](NC(=O)CCCCCN2C(=O)C=CC2=O)C(C)C)cc1)C(C)C]

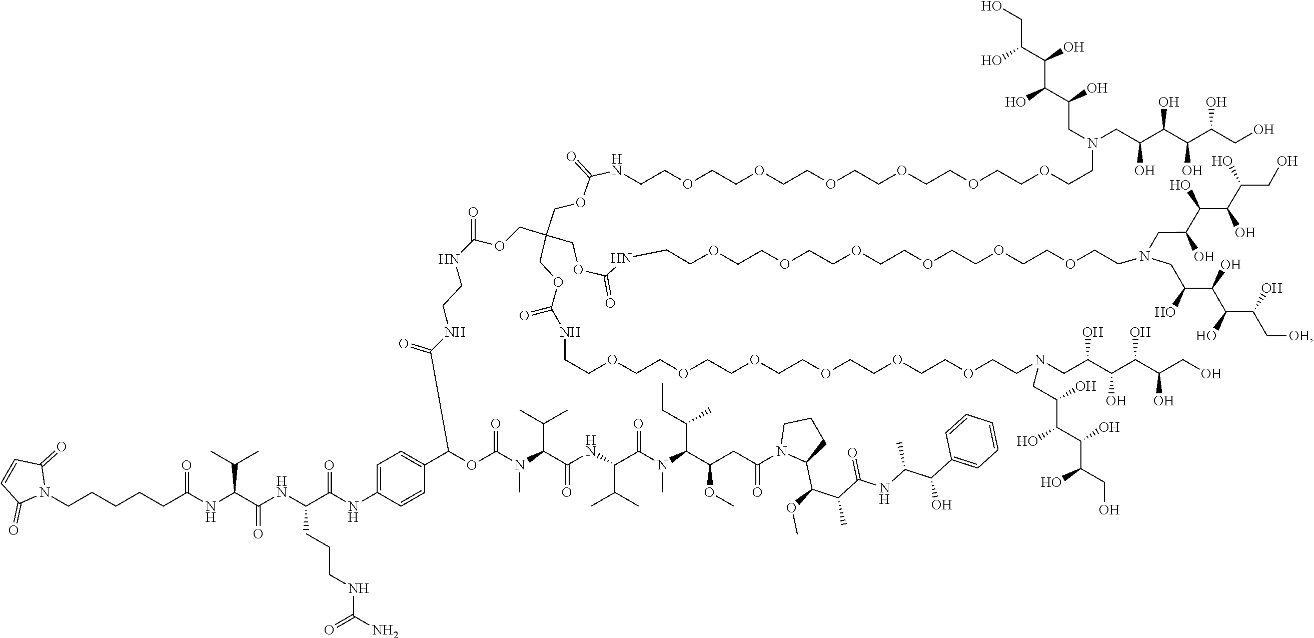

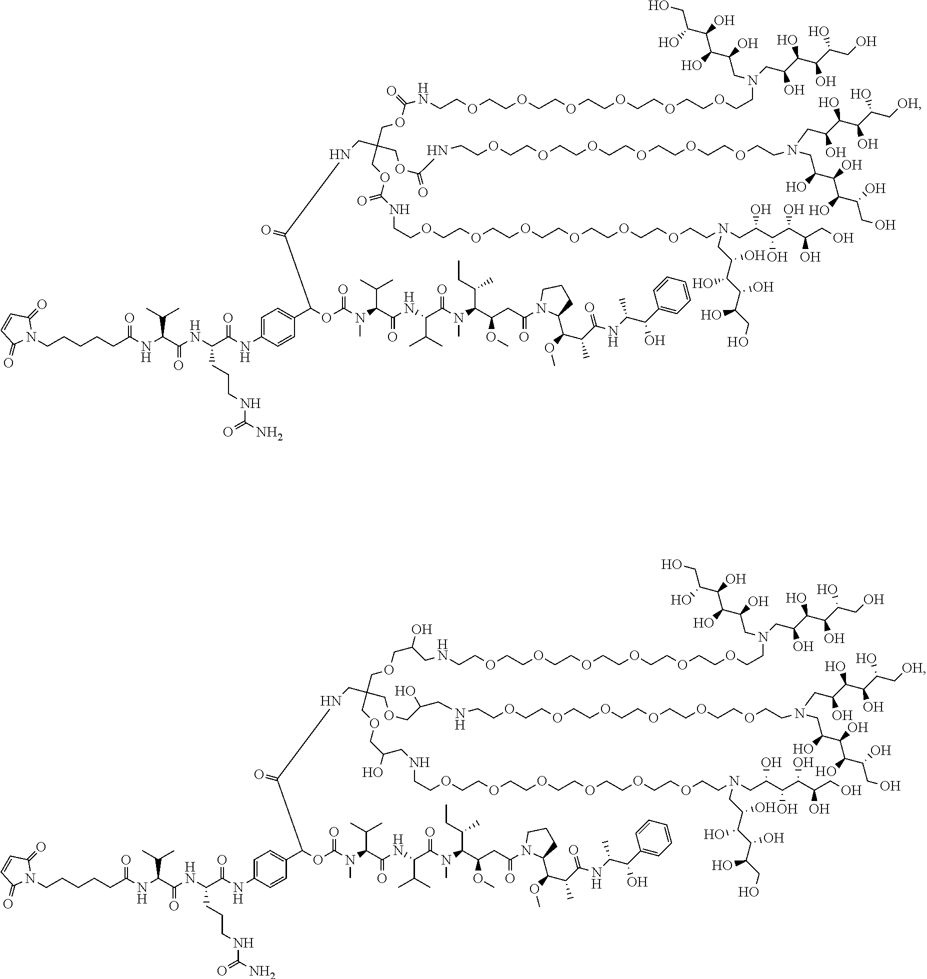

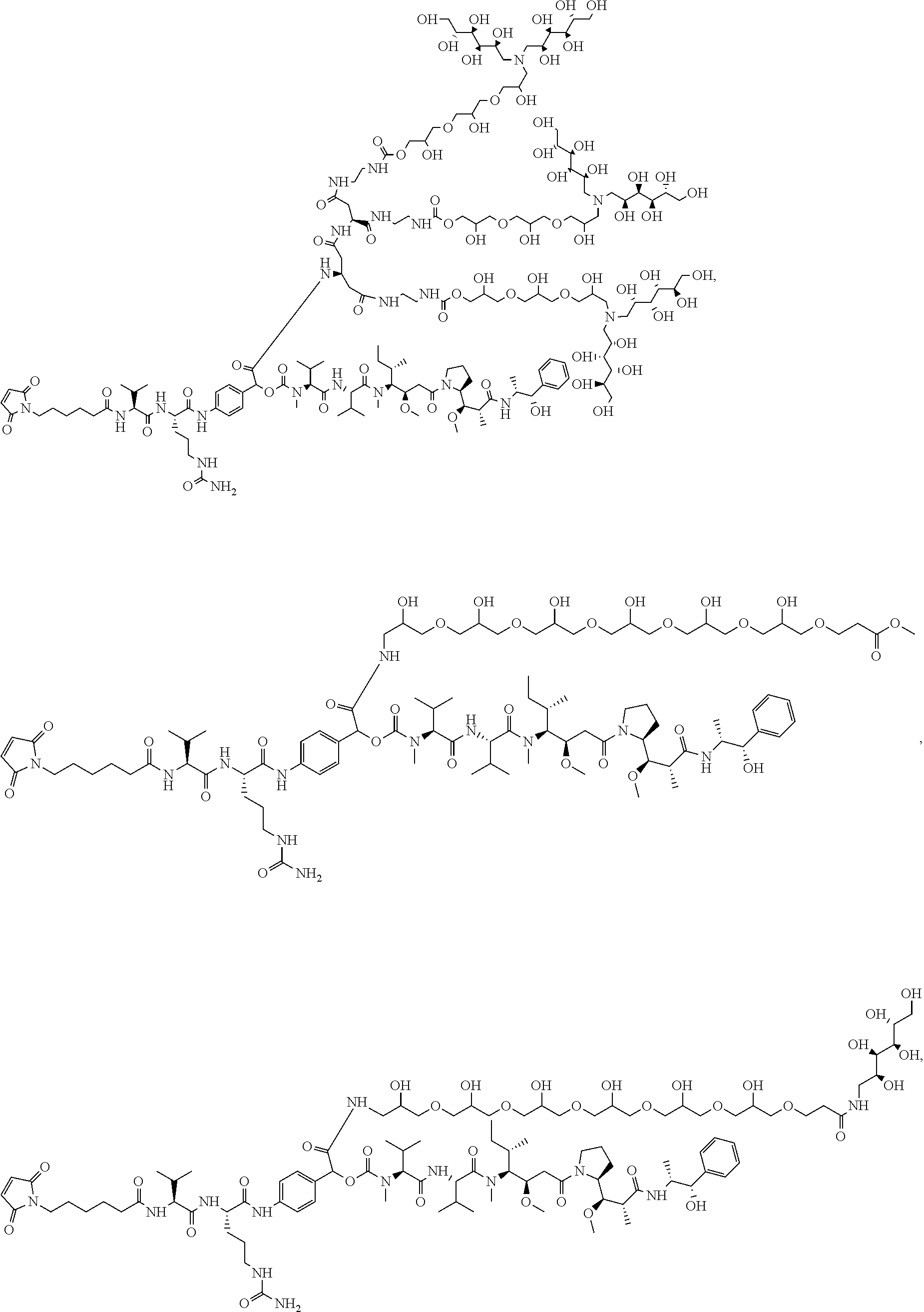

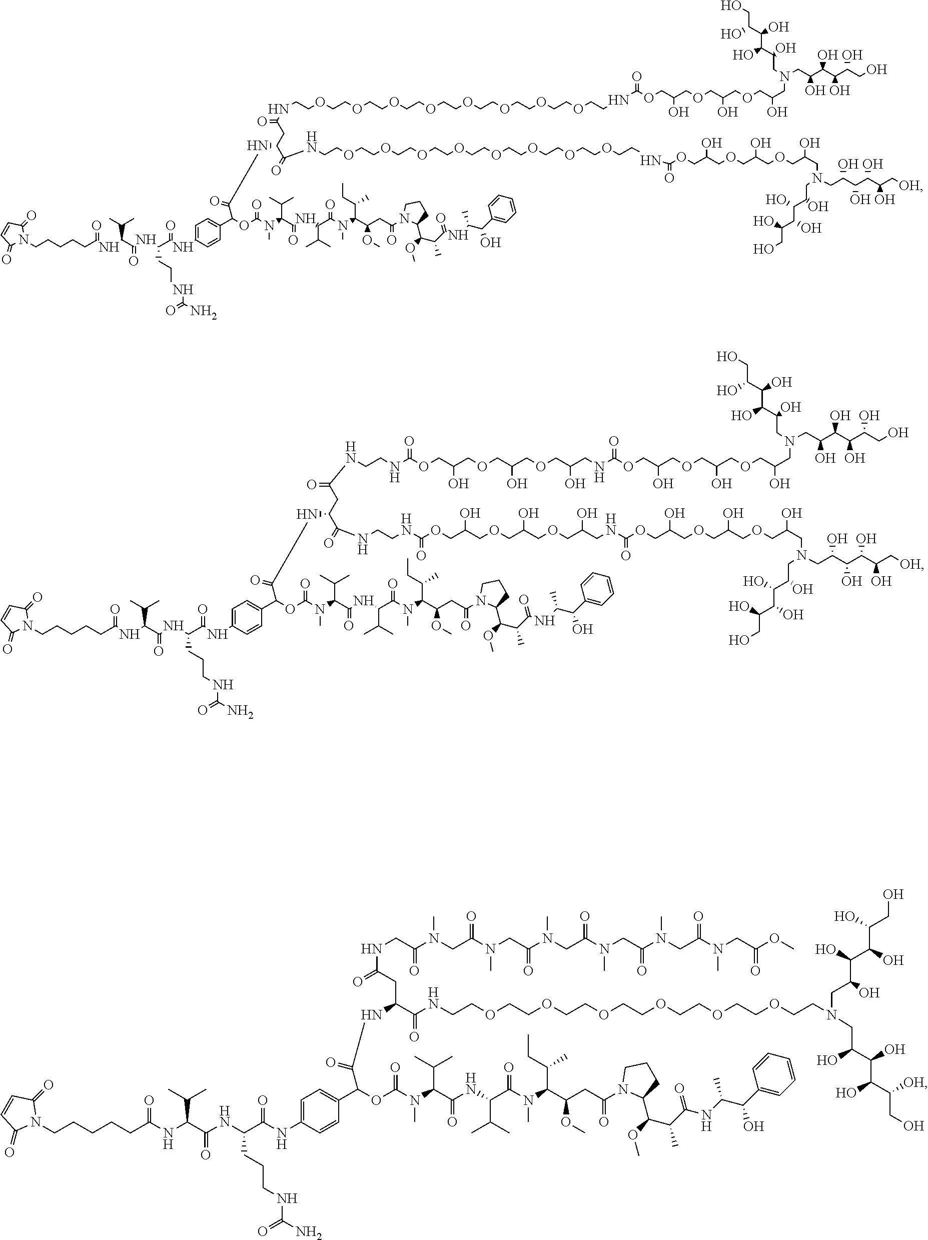

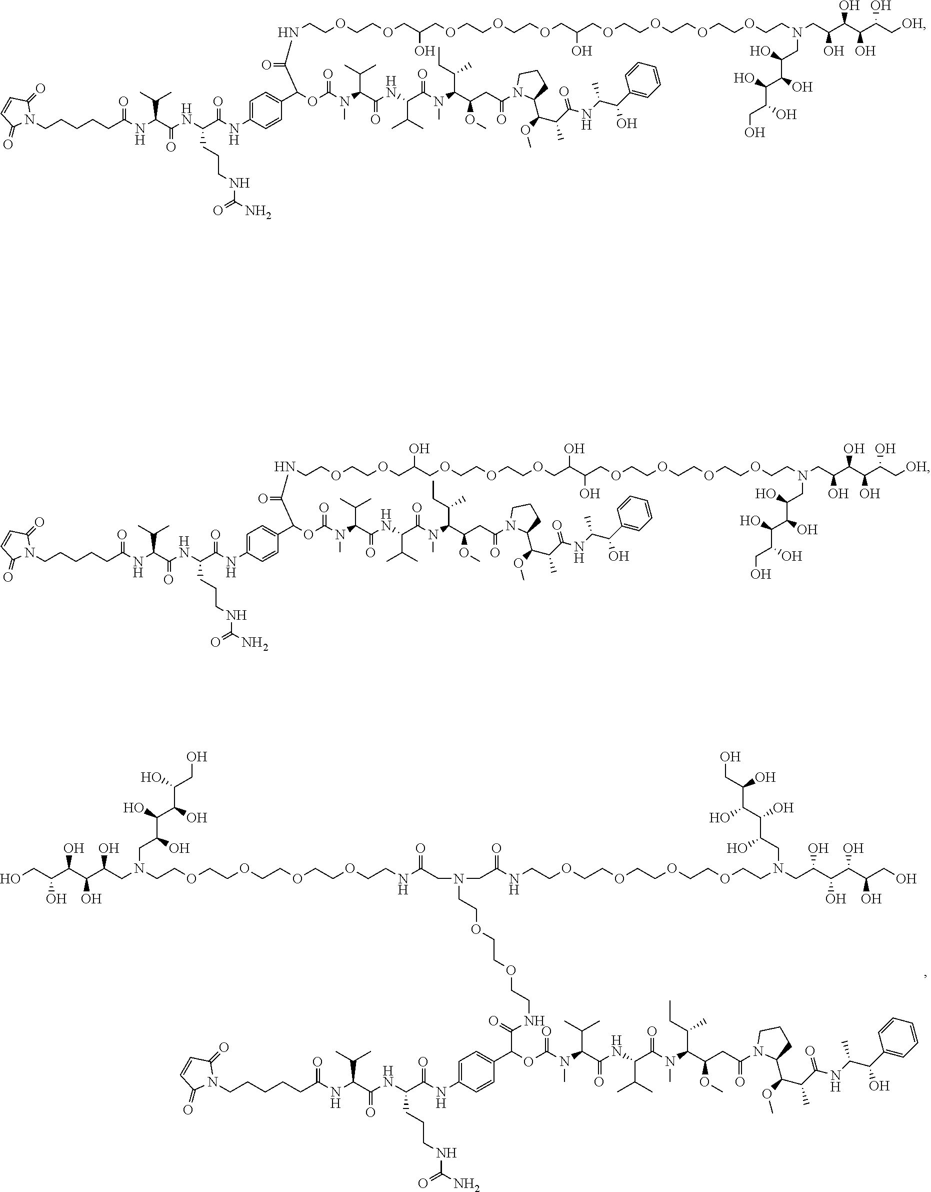

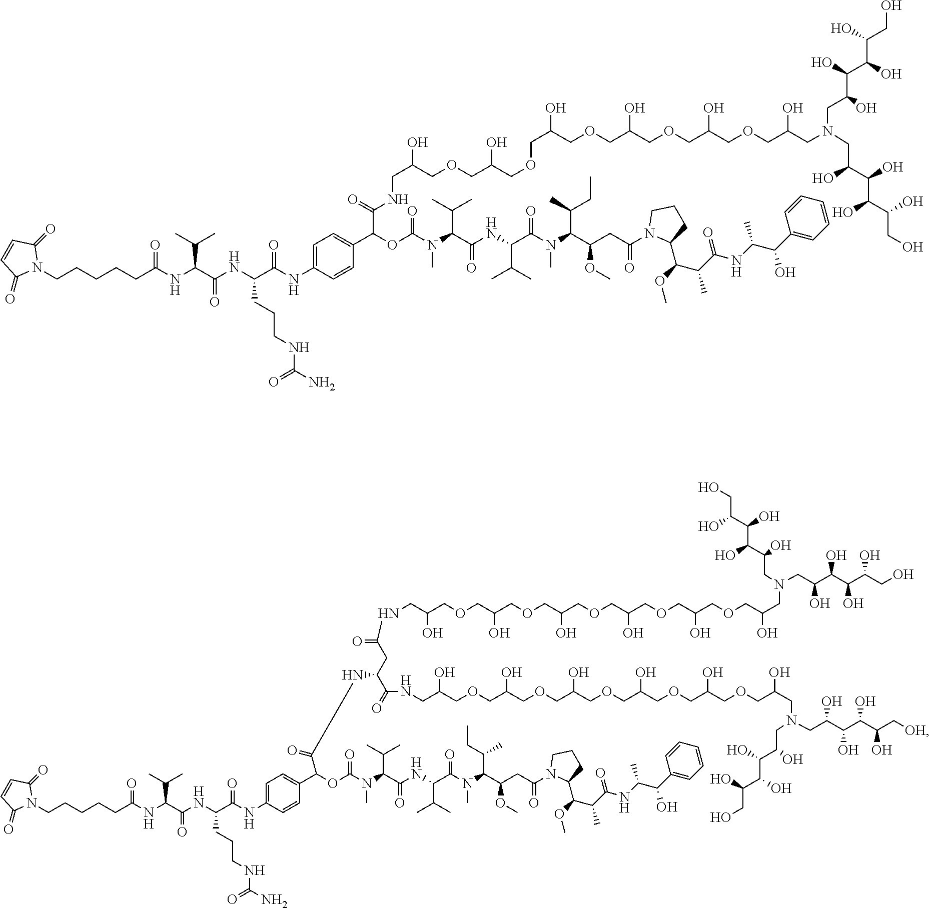

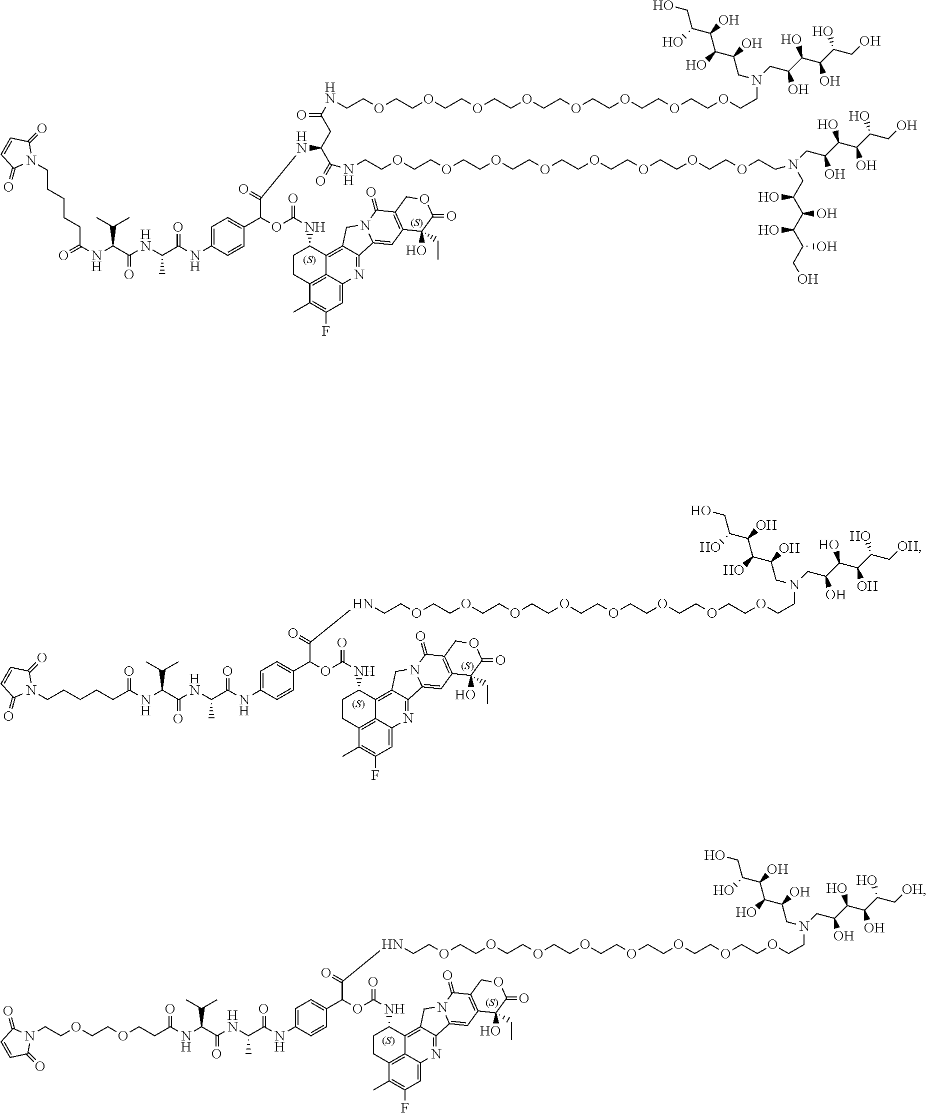

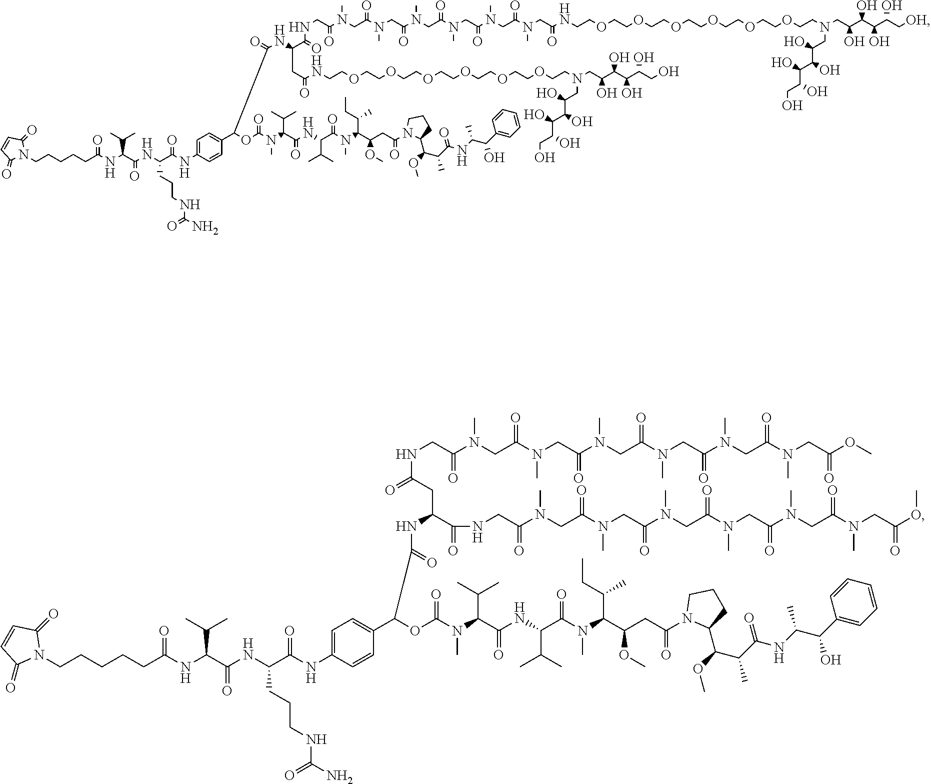

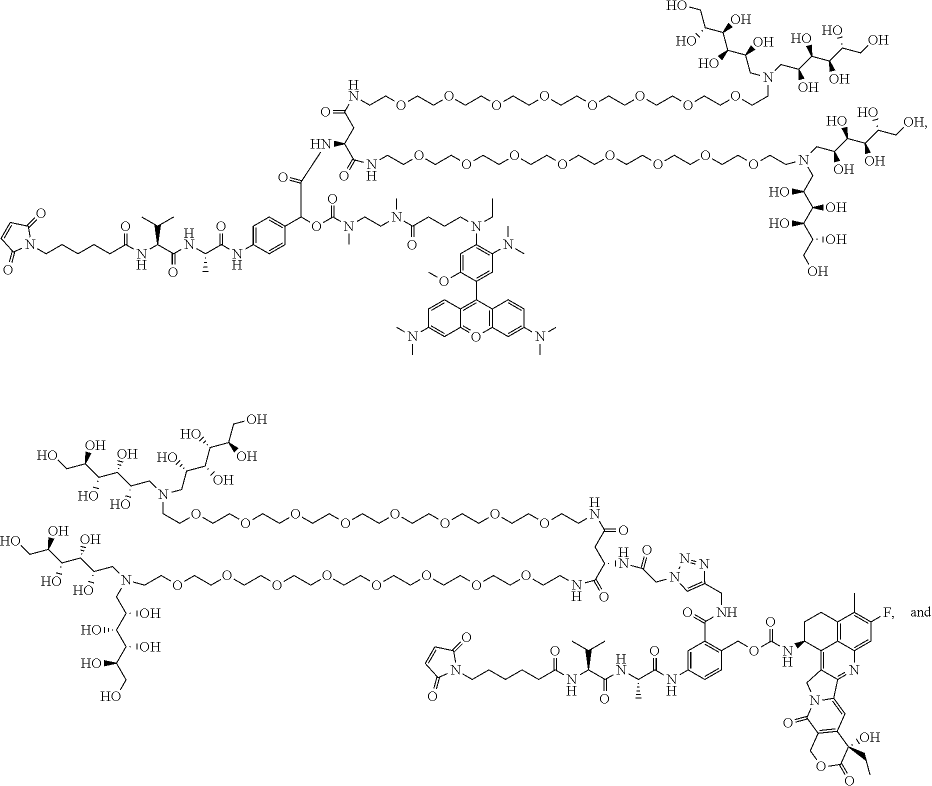

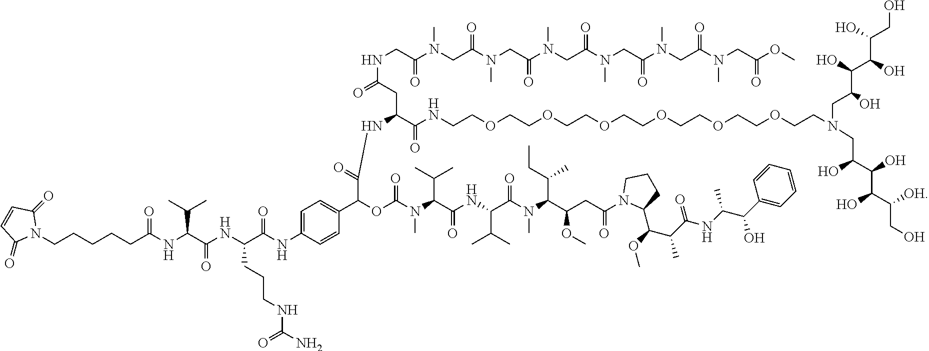 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@@H](CC(=O)NCC(=O)N(C)CC(=O)N(C)CC(=O)N(C)CC(=O)N(C)CC(=O)N(C)CC(=O)N(C)CC(=O)OC)C(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

In some embodiments, the Drug-Linker may be a stereoisomer of one of the above structures.

### Targeting Groups

In some embodiments, the Linkers are attached to Targeting groups (also described herein as “Targeting units”) to form Targeting unit-Linkers. In some embodiments, the Linkers are attached to Targeting groups via a Stretcher unit and to a Drug unit(s) via the attachment site δ to form a conjugate. In some embodiments, a Targeting group is a protein, polypeptide or peptide. The Targeting groups can be antibodies, antigen binding portions thereof or non-antibody targeting groups. Non-antibody targeting groups may also be referred to as non-antibody scaffolds.

In some embodiments, a Targeting group specifically binds to a target molecule. As used herein, “specifically binds” refers to the ability of a Targeting group (e.g., an antibody or portion thereof) described herein to bind to a target with a KD 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. Specific binding can be influenced by, for example, the affinity and avidity of the Targeting group and the concentration of target polypeptide. The person of ordinary skill in the art can determine appropriate conditions under which the antibodies, antibody binding portions and non-antibody scaffolds described herein selectively bind to a target using any suitable methods, such as titration of a binding agent in a suitable cell binding assay. A Targeting group specifically bound to its target is not displaced by a non-similar competitor. In certain embodiments, a Targeting unit is said to specifically bind to its target when it preferentially recognizes its target in a complex mixture of proteins and/or macromolecules.

As used herein, the term “antibody” refers to an immunoglobulin molecule and immunologically active portions of immunoglobulin molecules, i.e., molecules that contain an antigen binding site(s) that specifically bind(s) to a target antigen. The term generally refers to antibodies comprised of two immunoglobulin heavy chain variable regions and two immunoglobulin light chain variable regions including full length antibodies (having heavy and light chain constant regions).

Each heavy chain is typically composed of a variable region (abbreviated as a VH region) and a constant region. The heavy chain constant region may include three domains CH1, CH2 and CH3 and optionally a fourth domain, CH4. Each light chain is composed of a variable region (abbreviated as a VL region) and a constant region. The light chain constant region is a CL domain. The VH and VL regions may be further divided into hypervariable regions referred to as complementarity-determining regions (CDRs) and interspersed with conserved regions referred to as framework regions (FR). Each VH and VL region thus includes three CDRs and four FRs that are arranged from the N terminus to the C terminus in the following order: FR1, CDR1, FR2, CDR2, FR3, CDR3, and FR4. This structure is well known to those skilled in the art.

As used herein, an “antigen-binding portion” of an antibody refers to the portions of an antibody having VH and/or VL sequences of an antibody or the CDRs of an antibody and that specifically binds to the target antigen. Examples of antigen binding portions include a Fab, a Fab′, a F(ab′)₂, a Fv, a scFv, a disulfide linked Fv, a single domain antibody (also referred to as a VHH, VNAR, sdAb, or nanobody) or a diabody (see, e.g., Huston et al., Proc. Natl. Acad. Sci. U.S.A., 85, 5879-5883 (1988) and Bird et al., Science 242, 423-426 (1988), which are incorporated herein by reference). As used herein, the terms Fab, F(ab′)₂ and Fv refer to the following: (i) a Fab is a monovalent fragment composed of the VL, VH, CL and CH1 domains; (ii) a F(ab′)₂ is a bivalent fragment comprising two Fab fragments linked to one another in the hinge region via a disulfide bridge; and (iii) a Fv composed of the VL and VH domains. Although the two domains of the Fv fragment, namely VL and VH, are encoded by separate coding regions, they may further be linked to one another using a synthetic linker, e.g., a poly-G4S amino acid sequence (′(G₄S)ₙ, disclosed as SEQ ID NO: 1, wherein n=1 to 5), making it possible to prepare them as a single protein chain in which the VL and VH regions combine in order to form monovalent molecules (known as single chain Fv or scFv). The term “antigen-binding portion” of an antibody is also intended to include such single chain antibodies. Other forms of single chain antibodies such as “diabodies” are likewise included here. Diabodies are bivalent, bispecific antibodies in which VH and VL regions are expressed on a single polypeptide chain, but using a linker connecting the VH and VL regions that is too short for the two regions to be able to combine on the same chain, thereby forcing the VH and VL regions to pair with complementary regions of a different chain (VL and VH, respectively), and to form two antigen-binding sites (see, for example, Holliger, R, et al. (1993) Proc. Natl. Acad. Sci. USA 90:64446448; Poljak, R. J, et al. (1994) Structure 2:1121-1123).

A single-domain antibody is an antigen binding portion of an antibody containing a single monomeric variable antibody region. Single domains antibodies can be derived from the variable region of the antibody heavy chain from camelids (e.g., nanobodies or VHH portions). Furthermore, the term single-domain antibody includes an autonomous human heavy chain variable domain (aVH) or VNAR portions derived from sharks (see, e.g., Hasler et al., Mol. Immunol. 75:28-37, 2016).

Techniques for producing single domain antibodies (e.g., DABs or VHH) are known in the art, as disclosed for example in Cossins et al. (2006, Prot Express Purif 51:253-259) and Li et al. (Immunol. Lett. 188:89-95, 2017). Single domain antibodies may be obtained, for example, from camels, alpacas or llamas by standard immunization techniques. (See, e.g., Muyldermans et al., TIBS 26:230-235, 2001; Yau et al., J Immunol Methods 281:161-75, 2003; and Maass et al., J Immunol Methods 324:13-25, 2007.) A VHH may have potent antigen-binding capacity and can interact with novel epitopes that are inaccessible to conventional VH-VL pairs (see, e.g., Muyldermans et al., 2001). Alpaca serum IgG contains about 50% camelid heavy chain only IgG antibodies (HCAbs) (see, e.g., Maass et al., 2007). Alpacas may be immunized with antigens and VHHs can be isolated that bind to and neutralize a target antigen (see, e.g., Maass et al., 2007). PCR primers that amplify alpaca VHH coding sequences have been identified and may be used to construct alpaca VHH phage display libraries, which can be used for antibody fragment isolation by standard biopanning techniques well known in the art (see, e.g., Maass et al., 2007).

In some embodiments, the Targeting group is an antibody or antigen binding portion thereof is a bispecific or multispecific binding agent. Bispecific and multi-specific antibodies include the following: an scFv1-ScFv2, an ScFv1₂-Fc-scFv2₂, an IgG-scFv, a DVD-Ig, a triomab/quadroma, a two-in-one IgG, a scFv2-Fc, a TandAb, and an scFv-HSA-scFv. In some embodiments, an IgG-scFv is an IgG(H)-scFv, scFv-(H)IgG, IgG(L)-scFv, svFc-(L)IgG, 2scFV-IgG or IgG-2scFv. See, e.g., Brinkmann and Kontermann, MAbs 9(2):182-212 (2017); Wang et al., Antibodies, 2019, 8, 43; Dong et al., 2011, MAbs 3:273-88; Natsume et al., J. Biochem. 140(3):359-368, 2006; Cheal et al., Mol. Cancer Ther. 13(7):1803-1812, 2014; and Bates and Power, Antibodies, 2019, 8, 28.

In some embodiments, the Targeting group specifically binds to a cancer associated antigen such as CD19, CD20, CD30, CD33, CD38, CA125, HER2, MUC-1, prostate-specific membrane antigen (PSMA), CD44 surface adhesion molecule, mesothelin (MLSN), carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-A1, IL-13R-a2, GD2, 1p19q, ABL1, AKT1, ALK, APC, AR, ATM, BRAF, BRCA1, BRCA2, cKIT, cMET, CSF1R, CTNNB1, FGFR1, FGFR2, FLT3, GNA11, GNAQ, GNAS, HRAS, IDH1, IDH2, JAK2, KDR (VEGFR2), KRAS, MGMT, MGMT-Me, MLH1, MPL, NOTCHI, NRAS, PDGFRA, Pgp, PIK3CA, PR, PTEN, RET, RRM1, SMO, SPARC, TLE3, TOP2A, TOPO1, TP53, TS, TUBB3, VHL, CDH1, ERBB4, FBXW7, HNF1A, JAK3, NPM1, PTPN11, RB1, SMAD4, SMARCB1, STK1, MLH1, MSH2, MSH6, PMS2, ROS1, ERCC1, 5T4 (TPBG), B7-H3, CCR7, CD105, CD22, CD46, CD47, CD56, CD70, CD71, CD79b, CDH6, CLDN6, CLDN18.2, CLEC12A, DLL3, DR5, ERBB3 (HER3), EPCAM, FOLR1, IGF1R, IL2RA (CD25), IL3RA, ITGB6, LIV-1, LRRC15, mesothelin (MSLN), NaPi2b (SLC34A2), nectin-4, PTK7, ROR1, SEZ6, SLC44A4, SLITRK6, Tissue Factor (TF), TROP2 or B7-H4. According to the invention, the terms “cancer associated antigen,” “tumor antigen,” “tumor expressed antigen,” “cancer antigen,” “cancer associated antigen,” and “cancer expressed antigen” are equivalents and are used interchangeably herein.

In some embodiments, a Targeting group specifically binds to a target such as CD19, CD20, CD30, CD33, CD70, LIV-1 or EGFRv3.

In some embodiments, the Targeting group is an antibody (or fragment thereof) that binds to a target having a sequence as disclosed in Leuschner et al., US 2022/0048951 and/or Lerchen et al., US 2022/0016258. Non-limiting examples of monoclonal antibodies include rituximab (Rituxan®), trastuzumab (Herceptin®), pertuzumab (Perjeta®)), bevacizumab (Avastin®), ranibizumab (Lucentis®), cetuximab (Erbitux®), alemtuzumab (Campath®), panitumumab (Vectibix®), ibritumomab (Zevalin®), tositumomab (Bexxar®), ipilimumab, zalutumumab, dalotuzumab, figitumumab, ramucirumab, galiximab, farletuzumab, ocrelizumab, ofatumumab (Arzerra®), the CD20 antibodies 2F2 (HuMax-CD20), 7D8, IgM2C6, IgG1 2C6, 11B8, B1, 2H7, LT20, 1FS or AT80 (see Teeling et al., J. Immunol. 177:362-371 (2006)), daclizumab (Zenapax®), and anti-LHRH receptor antibodies such as clones A9E4, F1G4, AT2G7, GNRH03, GNRHR2, etc. which can be used in combination with, inter alia, a conjugate in accordance with the invention.

In some embodiments, a Targeting group is a non-antibody scaffold. Such non-antibody scaffolds include, for example, Affibodies, Affilins, Anticalins, Atrimers, Avimers, Bicyclic peptides, Cys-knots, DARPins, FN3 scaffolds (e.g., Adnectins, Centyrins, Pronectins, and Tn3), Fynomers, Kunitz domains and OBodies. (See, e.g., Vazquez-Lombardi et al., Drug Discovery Today 20(10):1271 (2015) and the references cited therein.) Such Non-antibody protein scaffolds include, for example, Affibodies, Affilins, Anticalins, Atrimers, Avimers, Bicyclic peptides, Cys-knots, DARPins, FN3 scaffolds (e.g., Adnectins, Centyrins, Pronectins, and Tn3), Fynomers, Kunitz domains and OBodies. (See, e.g., Vazquez-Lombardi et al., Drug Discovery Today 20(10):1271 (2015) and the references cited therein.) Non-antibody scaffolds can be considered to fall into two structural categories, domain-sized constructs (in the range of 6 to 20 kDa), and constrained peptides (in the 2-4 kDa range). Domain-sized non-antibody scaffolds include, but are not limited to, affibodies, affilins, anticalins, atrimers, DARPins, FN3 scaffolds (such as adnectins and centyrins), fynomers, Kunitz domains, pronectins and OBodies. Peptide-sized non-antibody scaffolds include, for example, avimers, bicyclic peptides and cysteine knots. Non-antibody protein scaffolds can be considered to fall into two structural categories, domain-sized constructs (in the range of 6 to 20 kDa), and constrained peptides (in the 2-4 kDa range). Domain-sized non-antibody scaffolds include, but are not limited to, affibodies, affilins, anticalins, atrimers, DARPins, FN3 scaffolds (such as adnectins and centyrins), fynomers, Kunitz domains, pronectins and OBodies. Peptide-sized non-antibody scaffolds include, for example, avimers, bicyclic peptides and cysteine knots. These non-antibody scaffolds and the underlying proteins or peptides on which they are based or from which they have been derived are reviewed by, e.g., Simeon and Chen, Protein Cell 9(1): 3-14 (2018); Vazquez-Lombardi et al., Drug Discovery Today 20: 1271-1283 (2015), and by Binz et al., Nature Biotechnol. 23: 1257-1268 (2005), the contents of each of which are herein incorporated by reference in their entireties.

Advantages of using non-antibody scaffolds include increased affinity, target neutralization, and stability. Various non-antibody scaffolds also can overcome some of the limitations of antibody scaffolds, e.g., in terms of tissue penetration, smaller size, and thermostability. Some non-antibody scaffolds can also permit easier construction, not being hindered, for example, by potential light chain association concerns when bispecific constructs are desired. Methods of constructing constructs on a non-antibody scaffold are known to those of ordinary skill in the art.

Accordingly, in some embodiments, a Targeting group can comprise a non-antibody scaffold. Accordingly, in some embodiments, a Targeting group can comprise a non-antibody scaffold protein. One of skill in the art would appreciate that a Targeting group can include, in some embodiments, e.g., an adnectin scaffold or a portion derived from human tenth fibronectin type III domain (10Fn3); an anticalin scaffold derived from human lipocalin (e.g., such as those described in, e.g., WO2015/104406); an avimer scaffold or a protein fragment derived from the A-domain of low density-related protein (LRP) and/or very low density lipoprotein receptor (VLDLR); a fynomer scaffold or portion of the SH3 domain of FYN tyrosine kinase; a kunitz domain scaffold or portion of Kunitz-type protease inhibitors, such as a human trypsin inhibitor, aprotinin (bovine pancreatic trypsin inhibitor), Alzheimer's amyloid precursor protein, and tissue factor pathway inhibitor; a knottin scaffold (cysteine knot miniproteins), such as one based on a trypsin inhibitor from E. elaterium; an affibody scaffold or all or part of the Z domain of S. aureus protein A; a p3-Hairpin mimetic scaffold; a Designed ankyrin repeat protein (DARPin) scaffold or artificial protein scaffolds based on ankyrin repeat (AR) proteins; or any scaffold derived or based on human transferrin, human CTLA-4, human crystallin, and human ubiquitin. For example, the binding site of human transferrin for human transferrin receptor can be diversified to create a diverse library of transferrin variants, some of which have acquired affinity for different antigens. See, e.g., Ali et al. (1999) J. Biol. Chem. 274:24066-24073. The portion of human transferrin not involved with binding the receptor remains unchanged and serves as a scaffold, like framework regions of antibodies, to present the variant binding sites. The libraries are then screened, as an antibody library is, and in accordance with the methods described herein, against a target antigen of interest to identify those variants having optimal selectivity and affinity for the target antigen. See, e.g., Hey et al. (2005) TRENDS Biotechnol. 23(10):514-522.

### Constant Regions

In some embodiments, a Targeting group, such as an antibody or antigen-binding portion thereof or other Targeting group, has an antibody constant region(s). In some embodiments, the constant region is a fully human constant region(s). In some embodiments, the constant region is a humanized constant region(s). In some embodiments, the constant region is a non-human constant region(s). An immunoglobulin constant region refers to a heavy or light chain constant region. Human heavy chain and light chain constant region amino acid sequences are known in the art. A constant region can be of any suitable type, which can be selected from the classes of immunoglobulins, IgA, IgD, IgE, IgG, and IgM. Several immunoglobulin classes can be further divided into isotypes, e.g., IgG1, IgG2, IgG3, IgG4, or IgAI, and IgA2. The heavy-chain constant regions (Fc) that correspond to the different classes of immunoglobulins can be α, δ, ε, γ, and μ, respectively. The light chains can be one of either kappa (or κ) and lambda (or λ).

In some embodiments, a constant region can have an IgG isotype. In some embodiments, a constant region can have an IgG1 isotype. In some embodiments, a constant region can have an IgG2 isotype. In some embodiments, a constant region can have an IgG3 isotype. In some embodiments, a constant region can have an IgG4 isotype. In some embodiments, a constant region can have a hybrid isotype comprising constant regions from two or more isotypes. In some embodiments, an immunoglobulin constant region can be an IgG1 or IgG4 constant region. In some embodiments, a constant region is of the IgG1 isotype and has the amino acid sequence set forth in SEQ ID NO:2. In some embodiments, a constant region is of the kappa isotype and has the amino acid sequence set forth in SEQ ID NO:3.

Furthermore, a Targeting group comprising an antibody or an antigen-binding portion thereof or non-antibody scaffold may be part of a larger molecule formed by covalent or noncovalent association of the antibody or antigen binding portion with one or more other proteins or peptides. Relevant to such Targeting groups are the use, for example, of the streptavidin core region in order to prepare a tetrameric scFv molecule (Kipriyanov, S. M., et al. (1995), Human Antibodies and Hybridomas 6:93-101) and the use of a cysteine residue, a marker peptide and a C-terminal polyhistidinyl peptide, e.g. hexahistidinyl tag (‘hexahistidinyl tag’ disclosed as SEQ ID NO: 4) in order to produce bivalent and biotinylated scFv molecules (Kipriyanov, S. M., et al. (1994) Mol. Immunol. 31:10471058).

### Fc Domain Modifications to Alter Effector Function

In some embodiments, an Fc region or Fc domain of a Targeting group, such as an antibody or antigen binding portion thereof or non-antibody scaffold, has substantially no binding to at least one Fc receptor selected from FcyRI (CD64), FcyRIIA (CD32a), FcyRIIB (CD32b), FcyRIIIA (CD16a), and FcyRIIIB (CD16b). In some embodiments, an Fc region or domain exhibits substantially no binding to any of the Fc receptors selected from FcyRI (CD64), FcyRIIA (CD32a), FcyRIIB (CD32b), FcyRIIIA (CD16a), and FcyRIIIB (CD16b). As used herein, “substantially no binding” refers to weak to no binding to a selected Fcgamma receptor or receptors. In some embodiments, “substantially no binding” refers to a reduction in binding affinity (i.e., increase in Kd) to a Fc gamma receptor of at least 1000-fold. In some embodiments, an Fc domain or region is an Fc null. As used herein, an “Fc null” refers to an Fc region or Fc domain that exhibits weak to no binding to any of the Fcgamma receptors. In some embodiments, an Fc null domain or region exhibits a reduction in binding affinity (i.e., increase in Kd) to Fc gamma receptors of at least 1000-fold.

In some embodiments, an Fc domain has reduced or substantially no effector function activity. As used herein, “effector function activity” refers to antibody dependent cellular cytotoxicity (ADCC), antibody dependent cellular phagocytosis (ADCP) and/or complement dependent cytotoxicity (CDC). In some embodiments, an Fc domain exhibits reduced ADCC, ADCP or CDC activity, as compared to a wildtype Fc domain. In some embodiments, an Fc domain exhibits a reduction in ADCC, ADCP and CDC, as compared to a wildtype Fc domain. In some embodiments, an Fc domain exhibits substantially no effector function (i.e., the ability to stimulate or effect ADCC, ADCP or CDC). As used herein, “substantially no effector function” refers to a reduction in effector function activity of at least 1000-fold, as compared to a wildtype or reference Fc domain.

In some embodiments, an Fc domain has reduced or no ADCC activity. As used herein reduced or no ADCC activity refers to a decrease in ADCC activity of an Fc domain by a factor of at least 10, at least 20, at least 30, at least 50, at least 100 or at least 500.

In some embodiments, an Fc domain has reduced or no CDC activity. As used herein reduced or no CDC activity refers to a decrease in CDC activity of an Fc domain by a factor of at least 10, at least 20, at least 30, at least 50, at least 100 or at least 500.

In vitro and/or in vivo cytotoxicity assays can be conducted to confirm the reduction/depletion of ADCC and/or CDC activity. For example, Fc receptor (FcR) binding assays can be conducted to ensure that the antibody lacks Fcgamma receptor binding (hence likely lacking ADCC activity). The primary cells for mediating ADCC, NK cells, express FcgammaRIII only, whereas monocytes express FcgammaRI, FcgammaRII and FcgammaRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest are described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assay methods may be employed (see, for example, ACTI™ non-radioactive cytotoxicity assay for flow cytometry (CellTechnology, Inc. Mountain View, Calif.; and CytoTox 96™ non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al., Proc. Nat'l Acad. Sci. USA 95:652-656 (1998).

C1q binding assays may also be carried out to confirm that an antibody or Fc domain or region is unable to bind C1q and hence lacks CDC activity or has reduced CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)).

In some embodiments, an Fc domain has reduced or no ADCP activity. As used herein reduced or no ADCP activity refers to a decrease in ADCP activity of an Fc domain by a factor of at least 10, at least 20, at least 30, at least 50, at least 100 or at least 500.

ADCP binding assays may also be carried out to confirm that an antibody or Fc domain or region lacks ADCP activity or has reduced ADCP activity. See, e.g., US20190079077 and US20190048078 and the references disclosed therein.

A Targeting group, such as an antibody or antigen binding portion thereof or non-antibody scaffold, with reduced effector function activity includes those with substitution of one or more of Fc region residues, such as, for example, 238, 265, 269, 270, 297, 327 and 329, according to the EU number of Kabat (see, e.g., U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine, according to the EU numbering of Kabat (see U.S. Pat. No. 7,332,581). Certain antibody variants with diminished binding to FcRs are also known. (See, e.g., U.S. Pat. No. 6,737,056; WO 2004/056312, and Shields et al., J. Biol. Chem. 9(2): 6591-6604 (2001).) A Targeting group, such as an antibody or antigen binding portion thereof or non-antibody scaffold, with diminished binding to FcRs can be prepared containing such amino acid modifications.

In some embodiments, a Targeting group, such as an antibody or antigen binding portion thereof or non-antibody scaffold, comprises an Fc domain or region with one or more amino acid substitutions which diminish FcgammaR binding, e.g., substitutions at positions 234 and 235 of the Fc region (EU numbering of residues). In some embodiments, the substitutions are L234A and L235A (LALA), according to the EU numbering of Kabat. In some embodiments, the Fc domain comprises D265A and/or P329G in an Fc region derived from a human IgG1 Fc region, according to the EU numbering of Kabat. In some embodiments, the substitutions are L234A, L235A and P329G (LALA-PG), according to the EU numbering of Kabat, in an Fc region derived from a human IgG1 Fc region. (See, e.g., WO 2012/130831). In some embodiments, the substitutions are L234A, L235A and D265A (LALA-DA) in an Fc region derived from a human IgG1 Fc region, according to the EU numbering of Kabat.

In some embodiments, alterations are made in the Fc region that result in altered (i.e., either diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

### Methods of Making Antibodies and Antigen Binding Portions and Other Targeting Groups

In various embodiments, Targeting groups such as antibodies and antigen binding portions thereof, can be produced in human, murine or other animal-derived cells lines. Recombinant DNA expression can be used to produce antibodies and antigen binding portions thereof. This allows the production of antibodies as well as a spectrum of antigen binding portions (including fusion proteins) in a host species of choice. The production of antibodies and antigen binding portions thereof in bacteria, yeast, transgenic animals and chicken eggs are also alternatives for cell-based production systems. The main advantages of transgenic animals are potential high yields from renewable sources.

Nucleic acid molecules encoding the amino acid sequence(s) of Targeting group, such as an antibody or antigen binding portion thereof can be prepared by a variety of methods known in the art. These methods include, but are not limited to, preparation of synthetic nucleotide sequences encoding of an antibody or antigen binding portion. In addition, oligonucleotide-mediated (or site-directed) mutagenesis, PCR-mediated mutagenesis, and cassette mutagenesis can be used to prepare nucleotide sequences encoding an antibody or antigen binding portion. A nucleic acid sequence encoding at least an antibody or antigen binding portion thereof, or a polypeptide thereof, as described herein, can be recombined with vector DNA in accordance with conventional techniques, such as, for example, blunt-ended or staggered-ended termini for ligation, restriction enzyme digestion to provide appropriate termini, filling in of cohesive ends as appropriate, alkaline phosphatase treatment to avoid undesirable joining, and ligation with appropriate ligases or other techniques known in the art. Techniques for such manipulations are disclosed, e.g., by Maniatis et al., Molecular Cloning, Lab. Manual (Cold Spring Harbor Lab. Press, NY, 1982 and 1989), and Ausubel et al., Current Protocols in Molecular Biology (John Wiley & Sons), 1987-1993, and can be used to construct nucleic acid sequences and vectors that encode an antibody or antigen binding portion thereof or a VH or VL polypeptide thereof.

As used herein, the terms “nucleic acid” or “nucleic acid sequence” or “polynucleotide sequence” or “nucleotide” refers to a polymeric molecule incorporating units of ribonucleic acid, deoxyribonucleic acid or an analog thereof. The nucleic acid can be either single-stranded or double-stranded. A single-stranded nucleic acid can be one strand nucleic acid of a denatured double-stranded DNA. In some embodiments, the nucleic acid can be a cDNA, e.g., a nucleic acid lacking introns.

A nucleic acid molecule, such as DNA, is said to be “capable of expressing” a polypeptide if it contains nucleotide sequences that contain transcriptional and translational regulatory information and such sequences are “operably linked” to nucleotide sequences that encode the polypeptide. An operable linkage is a linkage in which the regulatory DNA sequences and the DNA sequence sought to be expressed (e.g., an antibody or antigen binding portion thereof) are connected in such a way as to permit gene expression of a polypeptide(s) or antigen binding portions in recoverable amounts. The precise nature of the regulatory regions needed for gene expression may vary from organism to organism, as is well known in the analogous art. See, e.g., Sambrook et al., 1989; Ausubel et al., 1987-1993.

Accordingly, the expression of a Targeting group, such as an antibody or antigen-binding portion thereof, can occur in either prokaryotic or eukaryotic cells. Suitable hosts include bacterial or eukaryotic hosts, including yeast, insects, fungi, bird and mammalian cells either in vivo or in situ, or host cells of mammalian, insect, bird or yeast origin. The mammalian cell or tissue can be of human, primate, hamster, rabbit, rodent, cow, pig, sheep, horse, goat, dog or cat origin, but other mammalian cells may be used. Further, by use of, for example, the yeast ubiquitin hydrolase system, in vivo synthesis of ubiquitin-transmembrane polypeptide fusion proteins can be accomplished. The fusion proteins so produced can be processed in vivo or purified and processed in vitro, allowing synthesis of an antibody or antigen binding portion thereof as described herein with a specified amino terminus sequence. Moreover, problems associated with retention of initiation codon-derived methionine residues in direct yeast (or bacterial) expression maybe avoided. (See, e.g., Sabin et al., 7 Bio/Technol. 705 (1989); Miller et al., 7 Bio/Technol. 698 (1989).) Any of a series of yeast gene expression systems incorporating promoter and termination elements from the actively expressed genes coding for glycolytic enzymes produced in large quantities when yeast is grown in medium rich in glucose can be utilized to obtain recombinant antibodies or antigen-binding portions thereof. Known glycolytic genes can also provide very efficient transcriptional control signals. For example, the promoter and terminator signals of the phosphoglycerate kinase gene can be utilized.

Production of antibodies or antigen-binding portions in insects can be achieved, for example, by infecting an insect host with a baculovirus engineered to express a polypeptide by methods known to those of ordinary skill in the art. See Ausubel et al., 1987-1993.

In some embodiments, the introduced nucleic acid sequence(s) (encoding an antibody or antigen binding portion thereof or a polypeptide thereof) is incorporated into a plasmid or viral vector capable of autonomous replication in a recipient host cell. Any of a wide variety of vectors can be employed for this purpose and are known and available to those of ordinary skill in the art. See, e.g., Ausubel et al., 1987-1993. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

Exemplary prokaryotic vectors known in the art include plasmids such as those capable of replication in E. coli. Other gene expression elements useful for the expression of DNA encoding antibodies or antigen-binding portions thereof include, but are not limited to (a) viral transcription promoters and their enhancer elements, such as the SV40 early promoter. (Okayama et al., 3 Mol. Cell. Biol. 280 (1983)), Rous sarcoma virus LTR (Gorman et al., 79 PNAS 6777 (1982)), and Moloney murine leukemia virus LTR (Grosschedl et al., 41 Cell 885 (1985)); (b) splice regions and polyadenylation sites such as those derived from the SV40 late region (Okayarea et al., 1983), and (c) polyadenylation sites such as in SV40 (Okayama et al., 1983). Immunoglobulin-encoding DNA genes can be expressed as described by Liu et al., infra, and Weidle et al., 51 Gene 21 (1987), using as expression elements the SV40 early promoter and its enhancer, the mouse immunoglobulin H chain promoter enhancers, SV40 late region mRNA splicing, rabbit S-globin intervening sequence, immunoglobulin and rabbit S-globin polyadenylation sites, and SV40 polyadenylation elements.

For immunoglobulin encoding nucleotide sequences, the transcriptional promoter can be, for example, human cytomegalovirus, the promoter enhancers can be cytomegalovirus and mouse/human immunoglobulin.

In some embodiments, for expression of DNA coding regions in rodent cells, the transcriptional promoter can be a viral LTR sequence, the transcriptional promoter enhancers can be either or both the mouse immunoglobulin heavy chain enhancer and the viral LTR enhancer, and the polyadenylation and transcription termination regions. In other embodiments, DNA sequences encoding other proteins are combined with the above-recited expression elements to achieve expression of the proteins in mammalian cells.

Each coding region or gene fusion is assembled in, or inserted into, an expression vector. Recipient cells capable of expressing the variable region(s) or antigen binding portions thereof are then transfected singly with nucleotides encoding an antibody or an antibody polypeptide or antigen-binding portion thereof, or are co-transfected with a polynucleotide(s) encoding VH and VL chain coding regions. The transfected recipient cells are cultured under conditions that permit expression of the incorporated coding regions and the expressed antibody chains or intact antibodies or antigen binding portions are recovered from the culture.

In some embodiments, the nucleic acids containing the coding regions encoding an antibody or antigen-binding portion thereof are assembled in separate expression vectors that are then used to co-transfect a recipient host cell. Each vector can contain one or more selectable genes. For example, in some embodiments, two selectable genes are used, a first selectable gene designed for selection in a bacterial system and a second selectable gene designed for selection in a eukaryotic system, wherein each vector has a set of coding regions. This strategy results in vectors which first direct the production, and permit amplification, of the nucleotide sequences in a bacterial system. The DNA vectors so produced and amplified in a bacterial host are subsequently used to co-transfect a eukaryotic cell, and allow selection of a co-transfected cell carrying the desired transfected nucleic acids (e.g., containing antibody heavy and light chains). Non-limiting examples of selectable genes for use in a bacterial system are the gene that confers resistance to ampicillin and the gene that confers resistance to chloramphenicol. Selectable genes for use in eukaryotic transfectants include the xanthine guanine phosphoribosyl transferase gene (designated gpt) and the phosphotransferase gene from Tn5 (designated neo). Alternatively, the fused nucleotide sequences encoding VH and VL chains can be assembled on the same expression vector.

For transfection of the expression vectors and production of antibodies or antigen binding portions thereof, the recipient cell line can be a Chinese Hamster ovary cell line (e.g., DG44) or a myeloma cell. Myeloma cells can synthesize, assemble and secrete immunoglobulins encoded by transfected immunoglobulin genes and possess the mechanism for glycosylation of the immunoglobulin. For example, in some embodiments, the recipient cell is the recombinant Ig-producing myeloma cell SP2/0. SP2/0 cells only produce immunoglobulins encoded by the transfected genes. Myeloma cells can be grown in culture or in the peritoneal cavity of a mouse, where secreted immunoglobulin can be obtained from ascites fluid.

An expression vector encoding an antibody or antigen-binding portion thereof can be introduced into an appropriate host cell by any of a variety of suitable means, including such biochemical means as transformation, transfection, protoplast fusion, calcium phosphate-precipitation, and application with polycations such as diethylaminoethyl (DEAE) dextran, and such mechanical means as electroporation, direct microinjection and microprojectile bombardment, as known to one of ordinary skill in the art. (See, e.g., Johnston et al., 240 Science 1538 (1988)).

Yeast provides certain advantages over bacteria for the production of immunoglobulin heavy and light chains. Yeasts carry out post-translational peptide modifications including glycosylation. A number of recombinant DNA strategies exist that utilize strong promoter sequences and high copy number plasmids which can be used for production of the desired proteins in yeast. Yeast recognizes leader sequences of cloned mammalian gene products and secretes polypeptides bearing leader sequences (i.e., pre-polypeptides). See, e.g., Hitzman et al., 11th Intl. Conf. Yeast, Genetics & Molec. Biol. (Montpelier, France, 1982).

Yeast gene expression systems can be routinely evaluated for the levels of production, secretion and the stability of antibodies, and assembled antibodies and antigen binding portions thereof. Various yeast gene expression systems incorporating promoter and termination elements from the actively expressed genes coding for glycolytic enzymes produced in large quantities when yeasts are grown in media rich in glucose can be utilized. Known glycolytic genes can also provide very efficient transcription control signals. For example, the promoter and terminator signals of the phosphoglycerate kinase (PGK) gene can be utilized. Another example is the translational elongation factor 1alpha promoter, such as that from Chinese hamster cells. A number of approaches can be taken for evaluating optimal expression plasmids for the expression of immunoglobulins in yeast. See II DNA Cloning 45, (Glover, ed., IRL Press, 1985) and e.g., U.S. Publication No. US 2006/0270045 A1.

Bacterial strains can also be utilized as hosts for the production of the antibody molecules or antigen binding portions thereof as described herein. E. coli K12 strains such as E. coli W3110, Bacillus species, enterobacteria such as Salmonella typhimurium or Serratia marcescens, and various Pseudomonas species can be used. Plasmid vectors containing replicon and control sequences that are derived from species compatible with a host cell are used in connection with these bacterial hosts. The vector carries a replication site, as well as specific genes which are capable of providing phenotypic selection in transformed cells. A number of approaches can be taken for evaluating the expression plasmids for the production of antibodies and antigen binding portions thereof in bacteria (see Glover, 1985; Ausubel, 1987, 1993; Sambrook, 1989; Colligan, 1992-1996).

Host mammalian cells can be grown in vitro or in vivo. Mammalian cells provide post-translational modifications to immunoglobulin molecules including leader peptide removal, folding and assembly of VH and VL chains, glycosylation of the antibody molecules, and secretion of functional antibody and/or antigen binding portions thereof.

Mammalian cells which can be useful as hosts for the production of antibody proteins, in addition to the cells of lymphoid origin described above, include cells of fibroblast origin, such as Vero or CHO-K1 cells. Exemplary eukaryotic cells that can be used to express immunoglobulin polypeptides include, but are not limited to, COS cells, including COS 7 cells; 293 cells, including 293-6E cells; CHO cells, including CHO—S and DG44 cells; PERC6™ cells (Crucell); and NSO cells. In some embodiments, a particular eukaryotic host cell is selected based on its ability to make desired post-translational modifications to the heavy chains and/or light chains. For example, in some embodiments, CHO cells produce polypeptides that have a higher level of sialylation than the same polypeptide produced in 293 cells.

In some embodiments, one or more antibodies or antigen-binding portions thereof can be produced in vivo in an animal that has been engineered or transfected with one or more nucleic acid molecules encoding the polypeptides, according to any suitable method.

In some embodiments, an antibody or antigen-binding portion thereof is produced in a cell-free system. Non-limiting exemplary cell-free systems are described, e.g., in Sitaraman et al., Methods Mol. Biol. 498: 229-44 (2009); Spirin, Trends Biotechnol. 22: 538-45 (2004); and Endo et al., Biotechnol. Adv. 21: 695-713 (2003).

Many vector systems are available for the expression of the VH and VL chains in mammalian cells (see Glover, 1985). Various approaches can be followed to obtain intact antibodies. As discussed above, it is possible to co-express VH and VL chains and optionally the associated constant regions in the same cells to achieve intracellular association and linkage of VH and VL chains into complete tetrameric H₂L₂ antibodies or antigen-binding portions thereof. The co-expression can occur by using either the same or different plasmids in the same host. Nucleic acids encoding the VH and VL chains or antigen binding portions thereof can be placed into the same plasmid, which is then transfected into cells, thereby selecting directly for cells that express both chains. Alternatively, cells can be transfected first with a plasmid encoding one chain, for example the VL chain, followed by transfection of the resulting cell line with a VH chain plasmid containing a second selectable marker. Cell lines producing antibodies or antigen-binding portions thereof via either route could be transfected with plasmids encoding additional copies of peptides, VH, VL, or VH plus VL chains in conjunction with additional selectable markers to generate cell lines with enhanced properties, such as higher production of assembled antibodies or antigen binding portions thereof or enhanced stability of the transfected cell lines.

Additionally, plants have emerged as a convenient, safe and economical alternative expression system for recombinant antibody production, which are based on large scale culture of microbes or animal cells. Antibodies or antigen binding portions thereof can be expressed in plant cell culture, or plants grown conventionally. The expression in plants may be systemic, limited to sub-cellular plastids, or limited to seeds (endosperms). See, e.g., U.S. Patent Pub. No. 2003/0167531; U.S. Pat. Nos. 6,080,560; 6,512,162; and WO 0129242. Several plant-derived antibodies have reached advanced stages of development, including clinical trials (see, e.g., Biolex, N.C.).

For intact antibodies, the variable regions (VH and VL regions) of antibodies are typically linked to at least a portion of an immunoglobulin constant region (Fc) or domain, typically that of a human immunoglobulin. Human constant region DNA sequences can be isolated in accordance with well-known procedures from a variety of human cells, such as immortalized B-cells (WO 87/02671). An antibody can contain both light chain and heavy chain constant regions. The heavy chain constant region can include CH1, hinge, CH2, CH3, and, optionally, CH4 regions. In some embodiments, the CH2 domain can be deleted or omitted.

Techniques described for the production of single chain antibodies (see, e.g. U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989); which are incorporated by reference herein in their entireties) can be adapted to produce single chain antibodies that specifically bind to the target antigen. Single chain antibodies are formed by linking the heavy and light chain variable regions of the Fv region via an amino acid bridge, resulting in a single chain polypeptide. Techniques for the assembly of functional Fv portions in E. coli can also be used (see, e.g. Skerra et al., Science 242:1038-1041 (1988); which is incorporated by reference herein in its entirety).

In some embodiments, an antigen binding portion comprises one or more scFvs. An scFv can be, for example, a fusion protein of the variable regions of the heavy (VH) and light chain (VL) variable regions of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are, e.g. described in Houston, J. S., Methods in Enzymol. 203 (1991) 46-96. Methods for making scFv molecules and designing suitable peptide linkers are described in, for example, U.S. Pat. Nos. 4,704,692; 4,946,778; Raag and Whitlow, FASEB 9:73-80 (1995) and Bird and Walker, TIBTECH, 9: 132-137 (1991). scFv-Fcs have been described by Sokolowska-Wedzina et al., Mol. Cancer Res. 15(8):1040-1050, 2017.

In some embodiments, an antigen binding portion is a single-domain antibody is an antibody portion consisting of a single monomeric variable antibody domain. Single domains antibodies can be derived from the variable domain of the antibody heavy chain from camelids (e.g., nanobodies or VHH portions). Furthermore, a single-domain antibody can be an autonomous human heavy chain variable domain (aVH) or VNAR portions derived from sharks (see, e.g., Hasler et al., Mol. Immunol. 75:28-37, 2016).

Techniques for producing single domain antibodies (DABs or VHH) are known in the art, as disclosed for example in Cossins et al. (2006, Prot Express Purif 51:253-259) and Li et al. (Immunol. Lett. 188:89-95, 2017). Single domain antibodies may be obtained, for example, from camels, alpacas or llamas by standard immunization techniques. (See, e.g., Muyldermans et al., TIBS 26:230-235, 2001; Yau et al., J Immunol Methods 281:161-75, 2003; and Maass et al., J Immunol Methods 324:13-25, 2007.) A VHH may have potent antigen-binding capacity and can interact with epitopes that are inaccessible to conventional VH-VL pairs (see, e.g., Muyldermans et al., 2001). Alpaca serum IgG contains about 50% camelid heavy chain only IgG antibodies (HCAbs) (see, e.g., Maass et al., 2007). Alpacas may be immunized with antigens and VHHs can be isolated that bind to and neutralize the target antigen (see, e.g., Maass et al., 2007). PCR primers that amplify alpaca VHH coding sequences have been identified and can be used to construct alpaca VHH phage display libraries, which can be used for antibody fragment isolation by standard biopanning techniques well known in the art (see, e.g., Maass et al., 2007).

Techniques for making multispecific antibodies include, but are not limited to, recombinant co-expression of two immunoglobulin heavy chain-light chain pairs having different specificities (see, e.g., Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168; Carter (2001), J Immunol Methods 248, 7-15). Multi-specific antibodies may also be made by engineering electrostatic steering effects for making antibody Fc-heterodimeric molecules (see, e.g., WO 2009/089004A1); cross-linking of two or more antibodies or antigen binding portions thereof (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody portions (see, e.g., Hollinger et al., Proc. Natl. Acad. Sci. USA, 90:6444-6448 (1993)); and using single-chain Fv (scFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” also can be Targeting groups (see, e.g. US 2006/0025576A1).

In some embodiments, the Targeting groups comprise different antigen-binding sites, fused to one or the other of the two subunits of the Fc domain; thus, the two subunits of the Fc domain may be comprised in two non-identical polypeptide chains. Recombinant co-expression of these polypeptides and subsequent dimerization leads to several possible combinations of the two polypeptides. To improve the yield and purity of the bispecific molecules in recombinant production, it will thus be advantageous to introduce in the Fc domain of the Targeting group a modification promoting the association of the desired polypeptides.

Generally, this method involves replacement of one or more amino acid residues at the interface of the two Fc domains by charged amino acid residues so that homodimer formation becomes electrostatically unfavorable but heterodimerization electrostatically favorable.

In some embodiments, a Targeting group is a “bispecific T cell engager” or BiTE (see, e.g., WO2004/106381, WO2005/061547, WO2007/042261, and WO2008/119567). This approach utilizes two antibody variable domains arranged on a single polypeptide. For example, a single polypeptide chain can include two single chain Fv (scFv) portions, each having a variable heavy chain (VH) and a variable light chain (VL) domain separated by a polypeptide linker of a length sufficient to allow intramolecular association between the two domains. This single polypeptide further includes a polypeptide spacer sequence between the two scFvs. Each scFv recognizes a different epitope, and these epitopes may be specific for different proteins, such that both proteins are bound by the BiTE.

As it is a single polypeptide, the bispecific T cell engager may be expressed using any prokaryotic or eukaryotic cell expression system known in the art, e.g., a CHO cell line. However, specific purification techniques (see, e.g., EP1691833) may be necessary to separate monomeric bispecific T cell engagers from other multimeric species, which may have biological activities other than the intended activity of the monomer. In one exemplary purification scheme, a solution containing secreted polypeptides is first subjected to a metal affinity chromatography, and polypeptides are eluted with a gradient of imidazole concentrations. This eluate is further purified using anion exchange chromatography, and polypeptides are eluted using with a gradient of sodium chloride concentrations. Finally, this eluate is subjected to size exclusion chromatography to separate monomers from multimeric species. In some embodiments, a Targeting group is a bispecific antibody is composed of a single polypeptide chain comprising two single chain FV portions (scFV) fused to each other by a peptide linker.

In some embodiments, a Targeting group is multispecific, such as an IgG-scFV. IgG-scFv formats include IgG(H)-scFv, scFv-(H)IgG, IgG(L)-scFv, svFc-(L)IgG, 2scFV-IgG and IgG-2scFv. These and other bispecific antibody formats and methods of making them have been described in for example, Brinkmann and Kontermann, MAbs 9(2):182-212 (2017); Wang et al., Antibodies, 2019, 8, 43; Dong et al., 2011, MAbs 3:273-88; Natsume et al., J. Biochem. 140(3):359-368, 2006; Cheal et al., Mol. Cancer Ther. 13(7):1803-1812, 2014; and Bates and Power, Antibodies, 2019, 8, 28.

Igg-like dual-variable domain antibodies (DVD-Ig) have been described by Wu et al., 2007, Nat Biotechnol 25:1290-97; Hasler et al., Mol. Immunol. 75:28-37, 2016 and in WO 08/024188 and WO 07/024715. Triomabs have been described by Chelius et al., MAbs 2(3):309-319, 2010. 2-in-1-IgGs have been described by Kontermann et al., Drug Discovery Today 20(7):838-847, 2015. Tanden antibody or TandAb have been described by Kontermann et al., id. ScFv-HSA-scFv antibodies have also been described by Kontermann et al. (id.).

Intact (e.g., whole) antibodies, their dimers, individual light and heavy chains, or antigen binding portions thereof can be recovered and purified by known techniques, e.g., immunoadsorption or immunoaffinity chromatography, chromatographic methods such as HPLC (high performance liquid chromatography), ammonium sulfate precipitation, gel electrophoresis, or any combination of these. See generally, Scopes, Protein Purification (Springer-Verlag, N.Y., 1982). Substantially pure antibodies or antigen binding portions thereof of at least about 90% to 95% homogeneity are advantageous, as are those with 98% to 99% or more homogeneity, particularly for pharmaceutical uses. Once purified, partially or to homogeneity as desired, an intact antibody or antigen binding portions thereof can then be used therapeutically or in developing and performing assay procedures, immunofluorescent staining, and the like. See generally, Vols. I & II Immunol. Meth. (Lefkovits & Pernis, eds., Acad. Press, NY, 1979 and 1981).

### Conjugates

In some embodiments, provided are Conjugates comprising a Targeting group as described herein attached to a Drug-Linker as described herein. In some embodiments, provided are Conjugates comprising a Targeting group as described herein attached to a Drug-Linker as described herein (e.g., Formula (A). For example, in some embodiments the Targeting group is selected from an antibody or an antigen-binding portion thereof. In more specific embodiments, the Targeting group is a monoclonal antibody, a Fab, a Fab′, an F(ab′), an Fv, a disulfide linked Fc, a scFv, a single domain antibody, a diabody, a bi-specific antibody, or a multi-specific antibody. In more specific embodiments, the Targeting group is a diabody, a DART, an anticalin, an affibody, an avimer, a DARPin, or an adnectin. In some embodiments, the Targeting group is mono-specific. In some embodiments, the Targeting group is bivalent. In some embodiments, the Targeting group is bispecific.

In some embodiments, provided are conjugates comprising a Targeting group attached to a Drug-Linker as described above, wherein the average drug loading (p^{load}) of the conjugate is from about 1 to about 8, about 2, about 4, about 6, about 8, about 10, about 12, about 14, about 16, about 3 to about 5, about 6 to about 8, or about 8 to about 16. In some cases, the p^{load} is about 1. In some cases, the p^{load} is about 2. In some cases, the p^{load} is about 4. In some cases, the p^{load} is about 8.

In some embodiments, provided a Conjugates selected from the following:

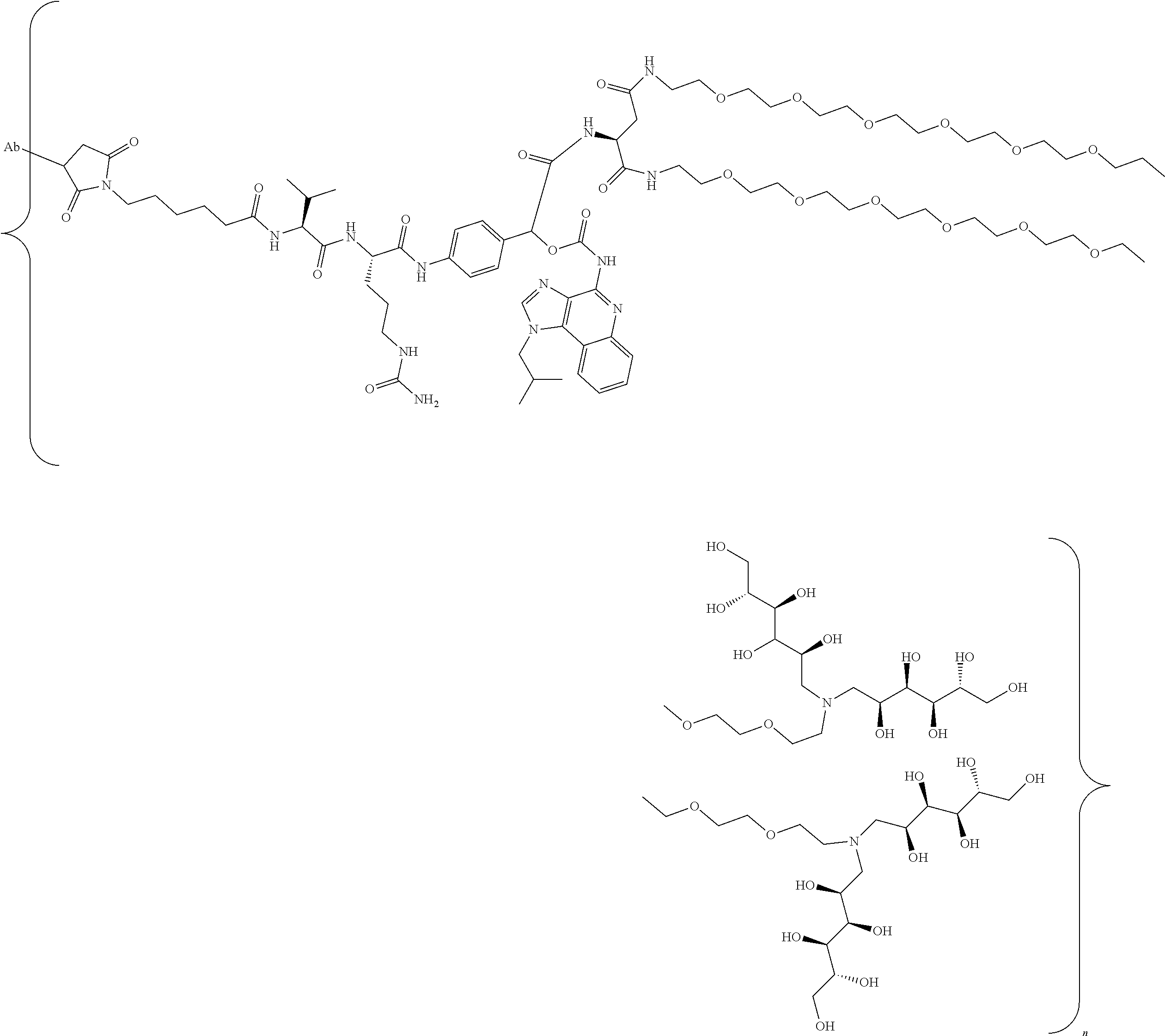

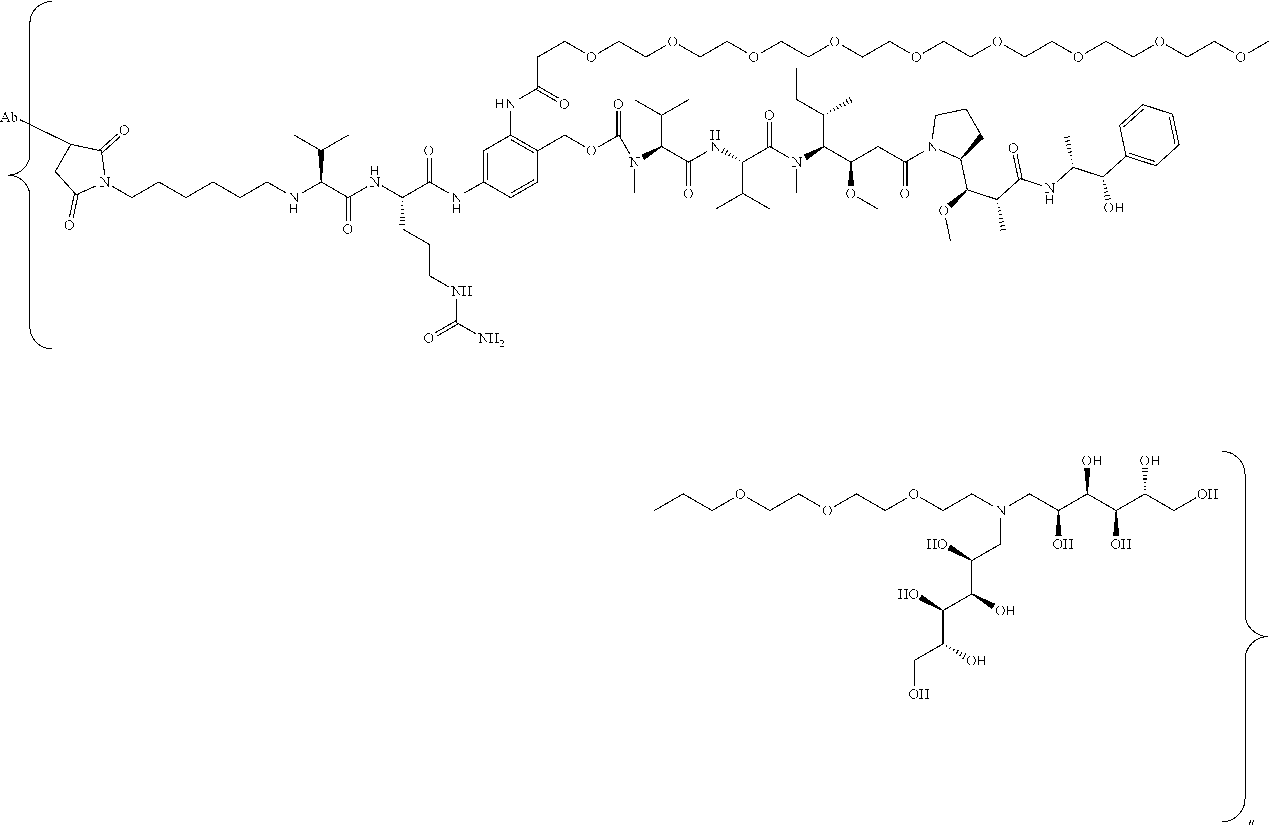

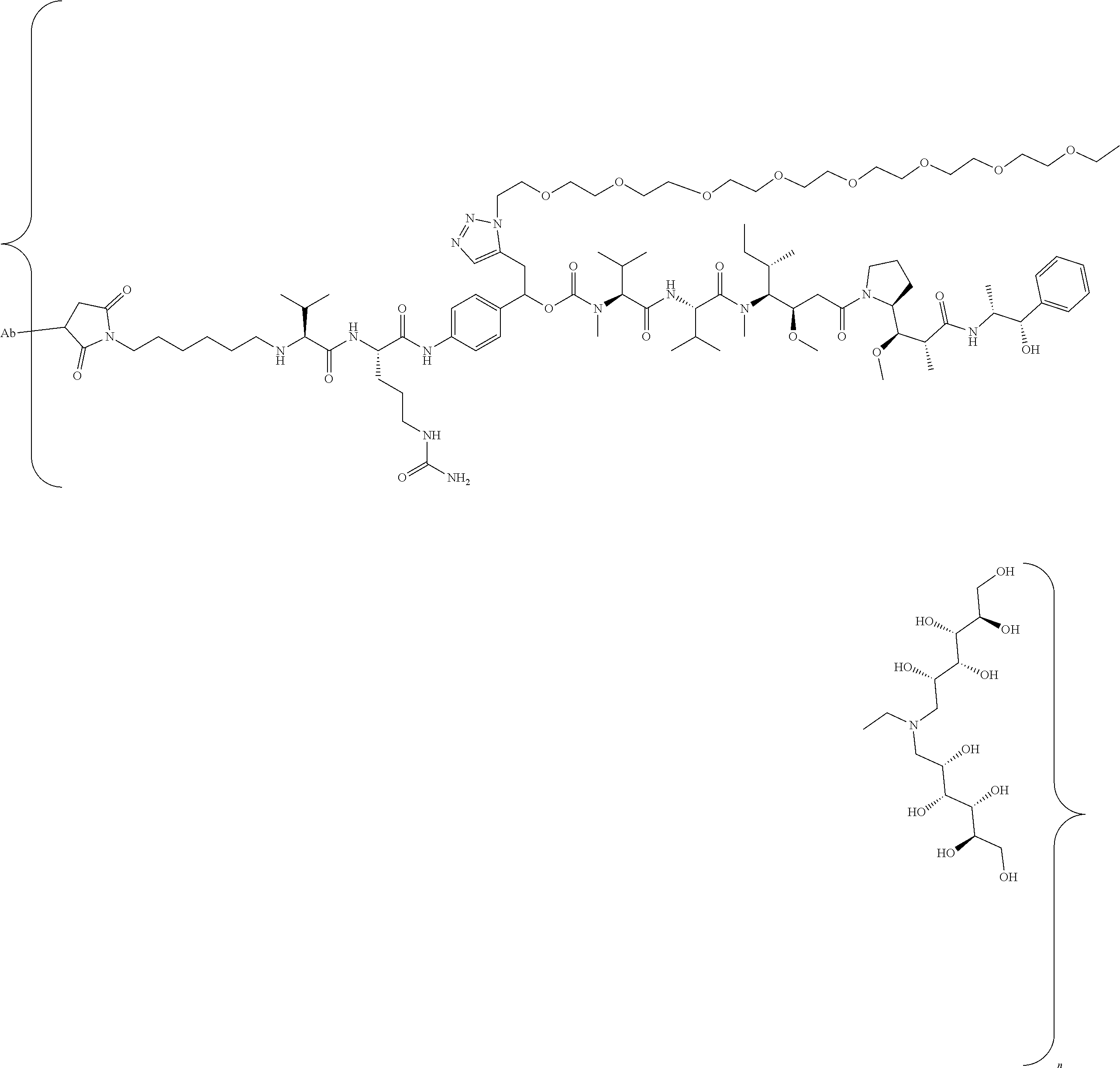

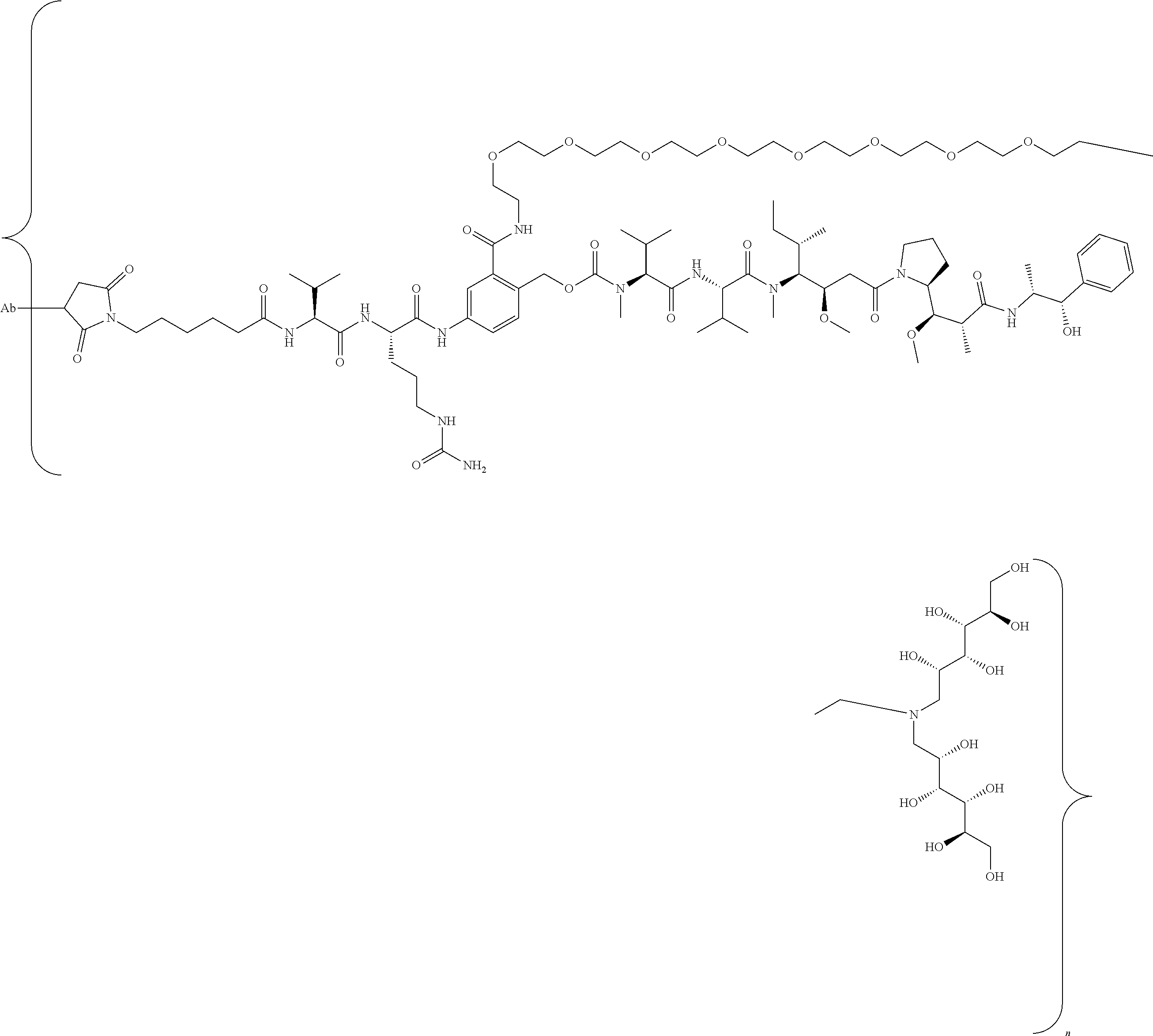

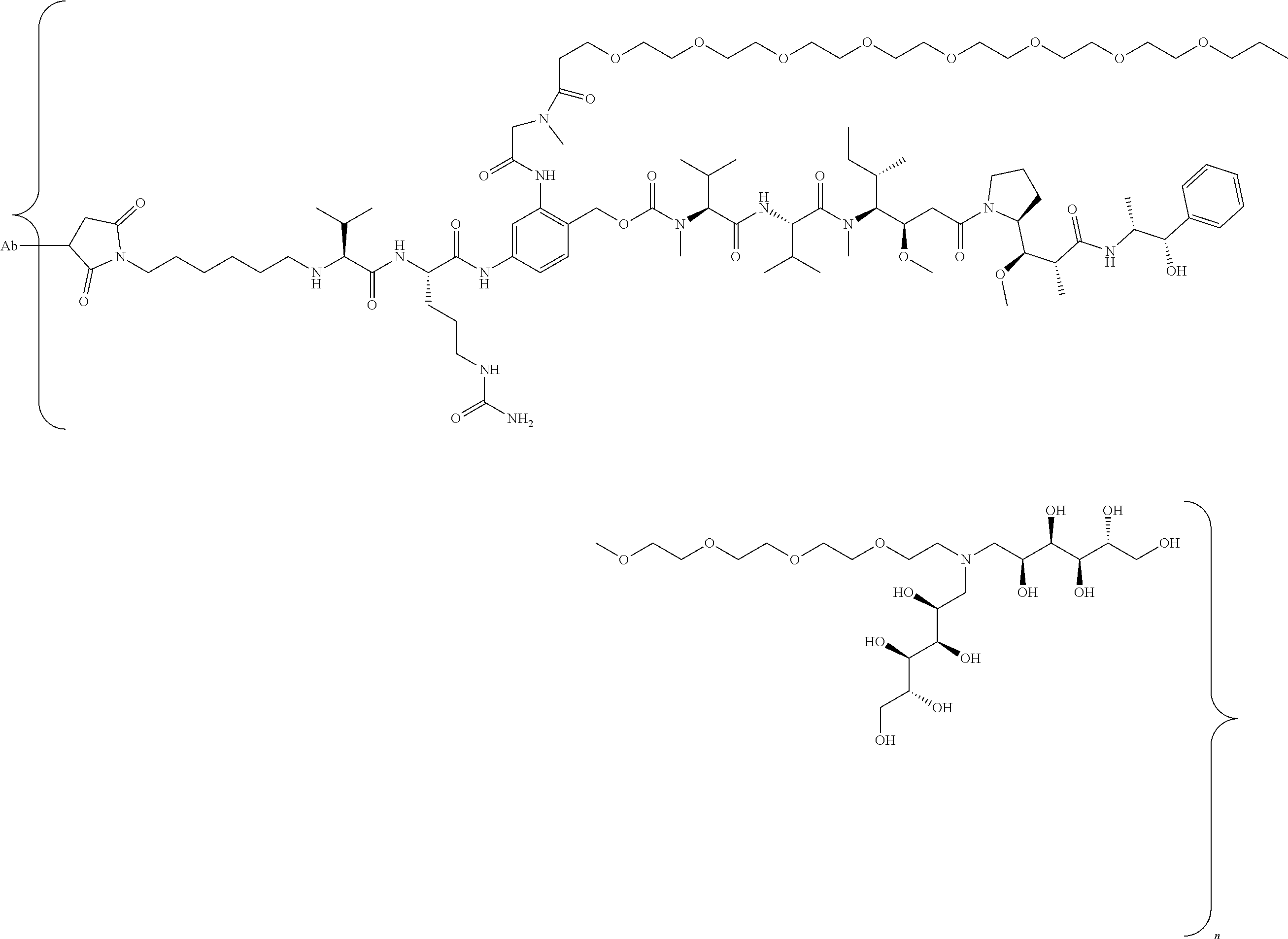

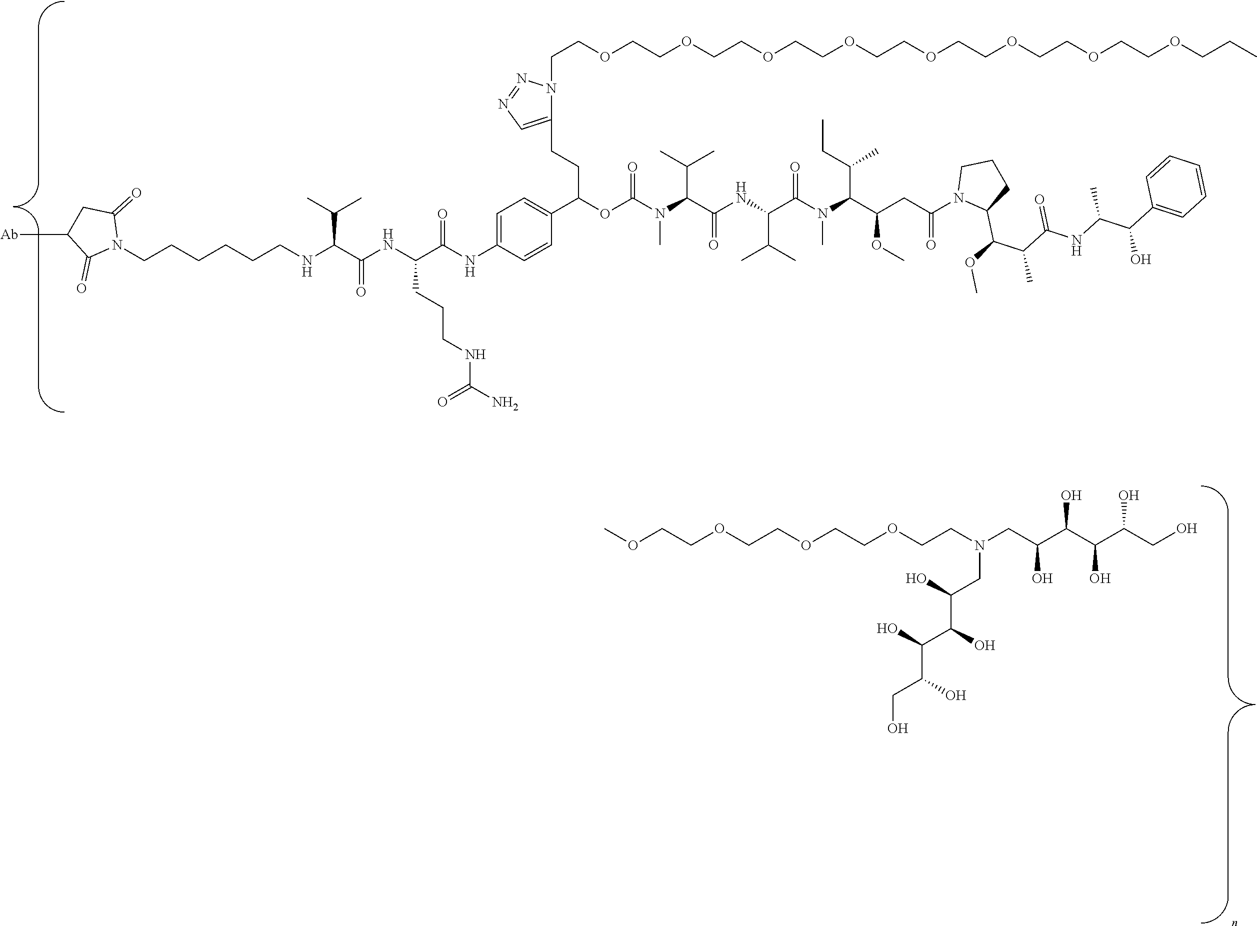

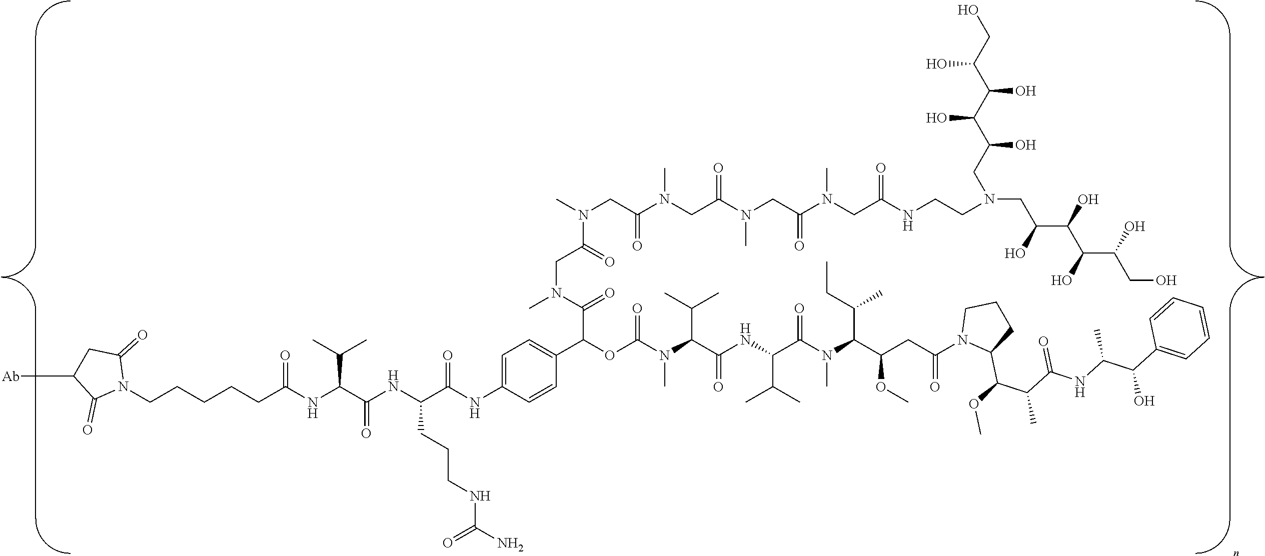

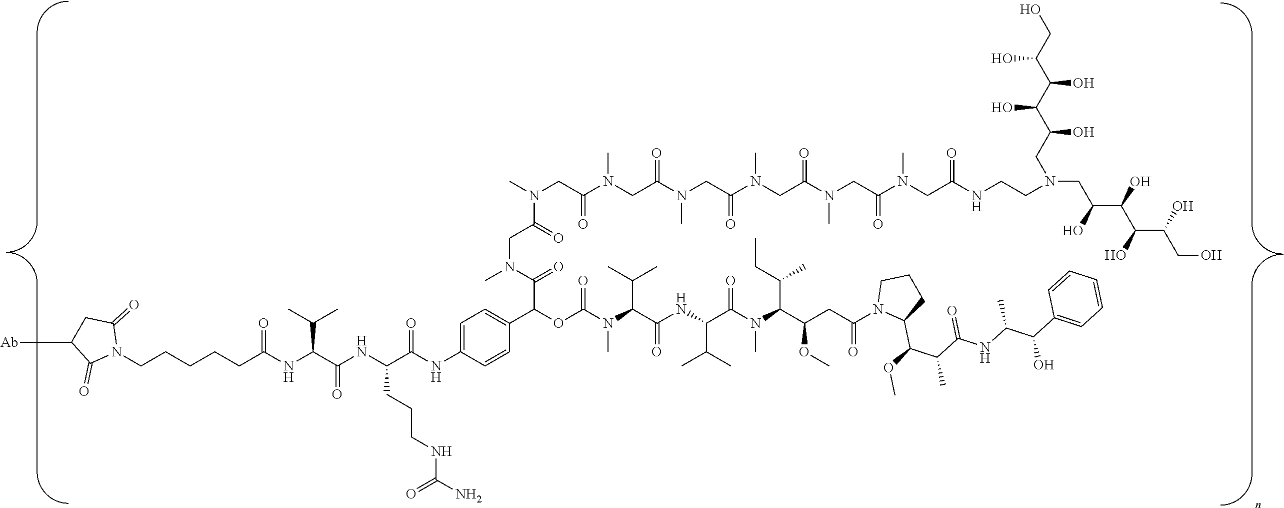

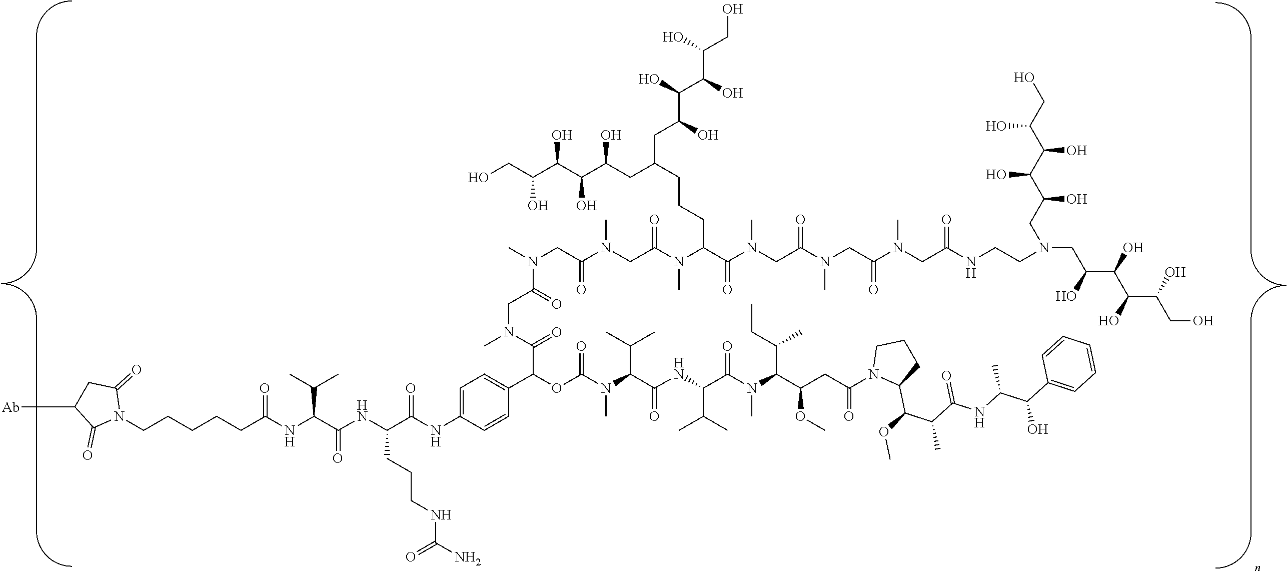

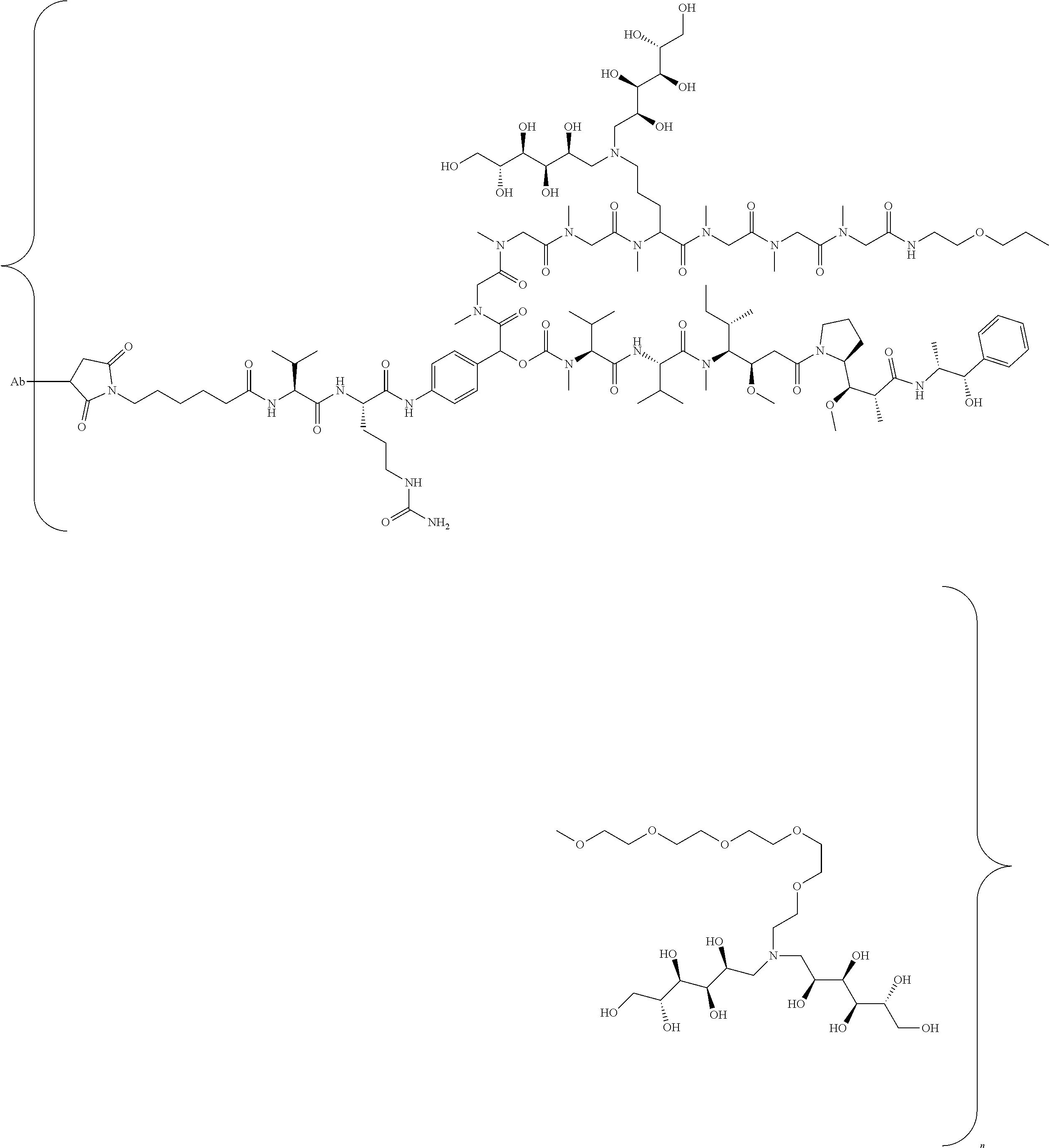

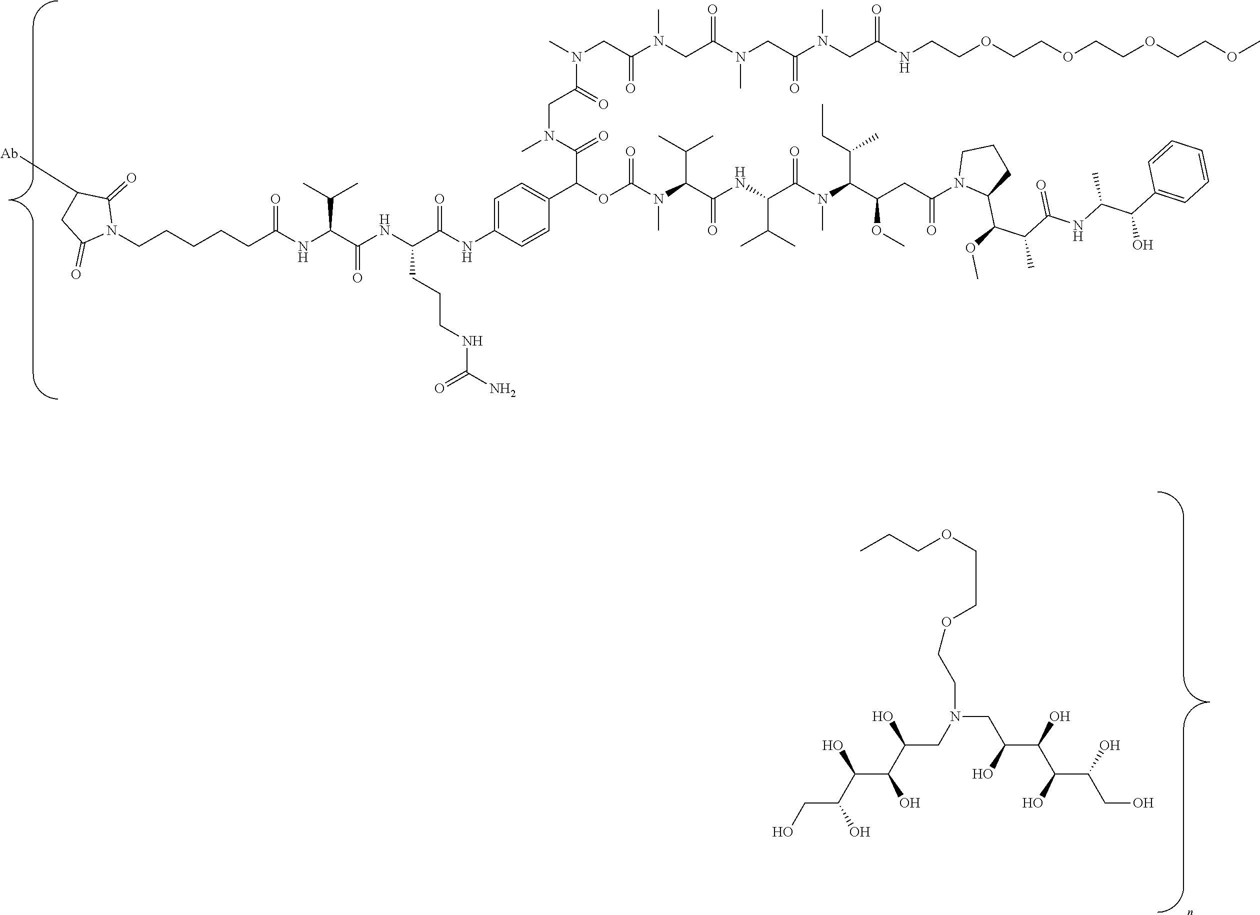

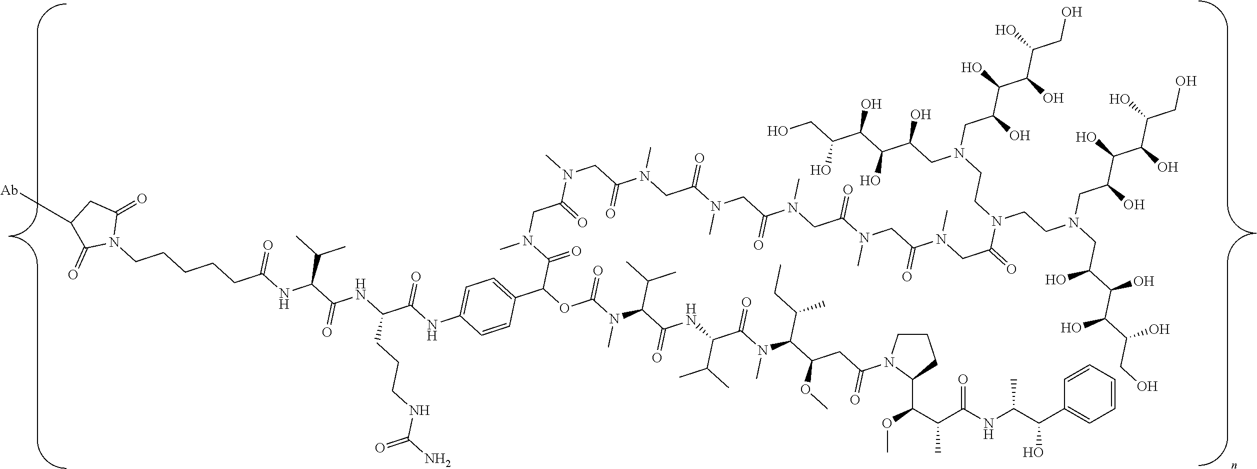

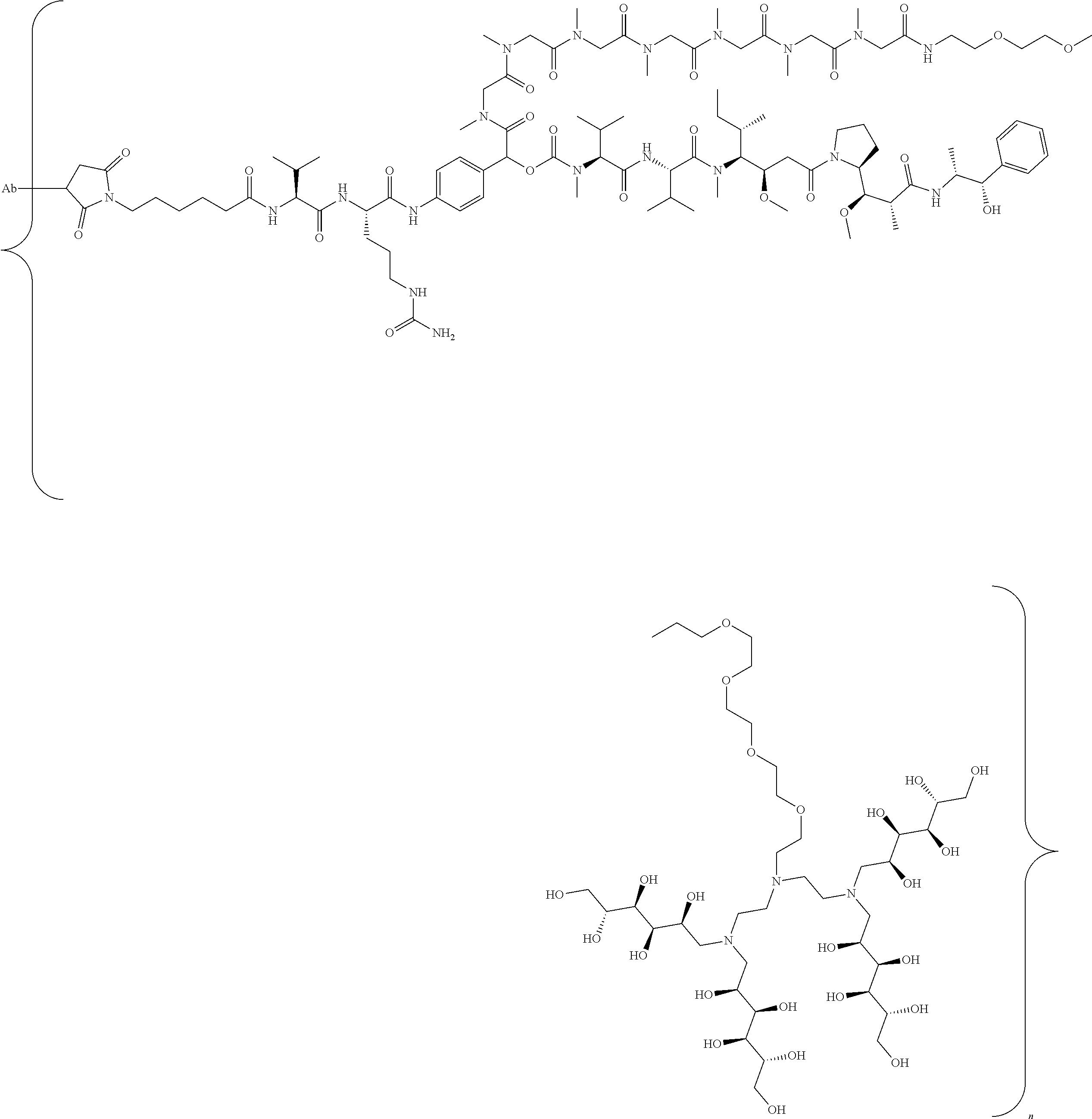

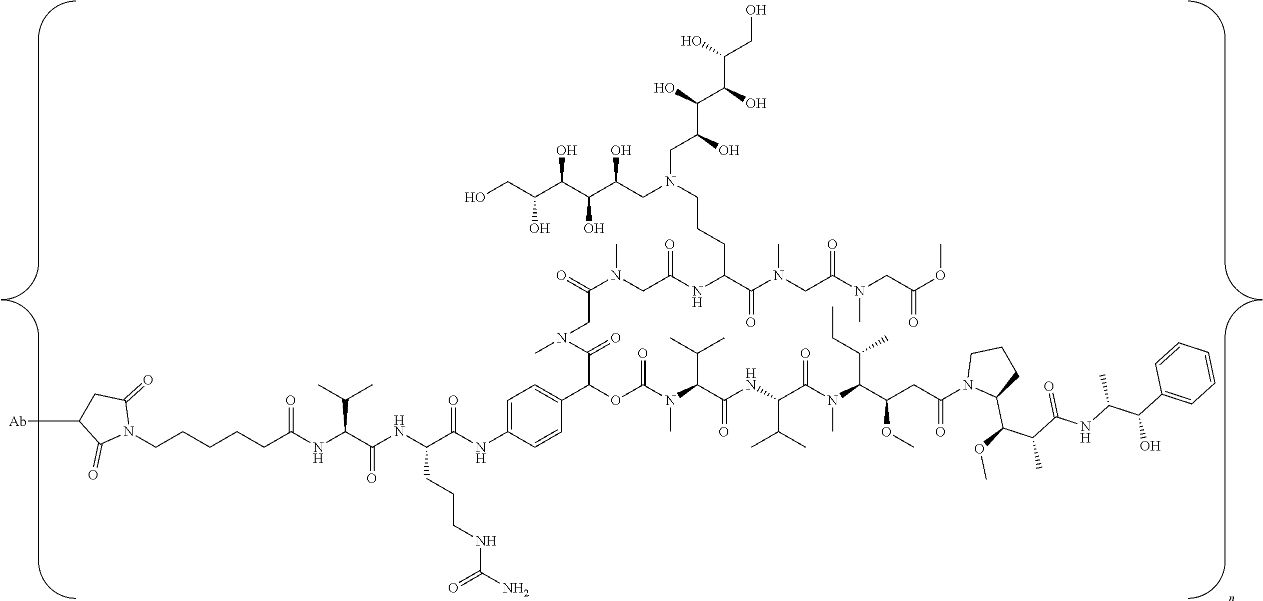

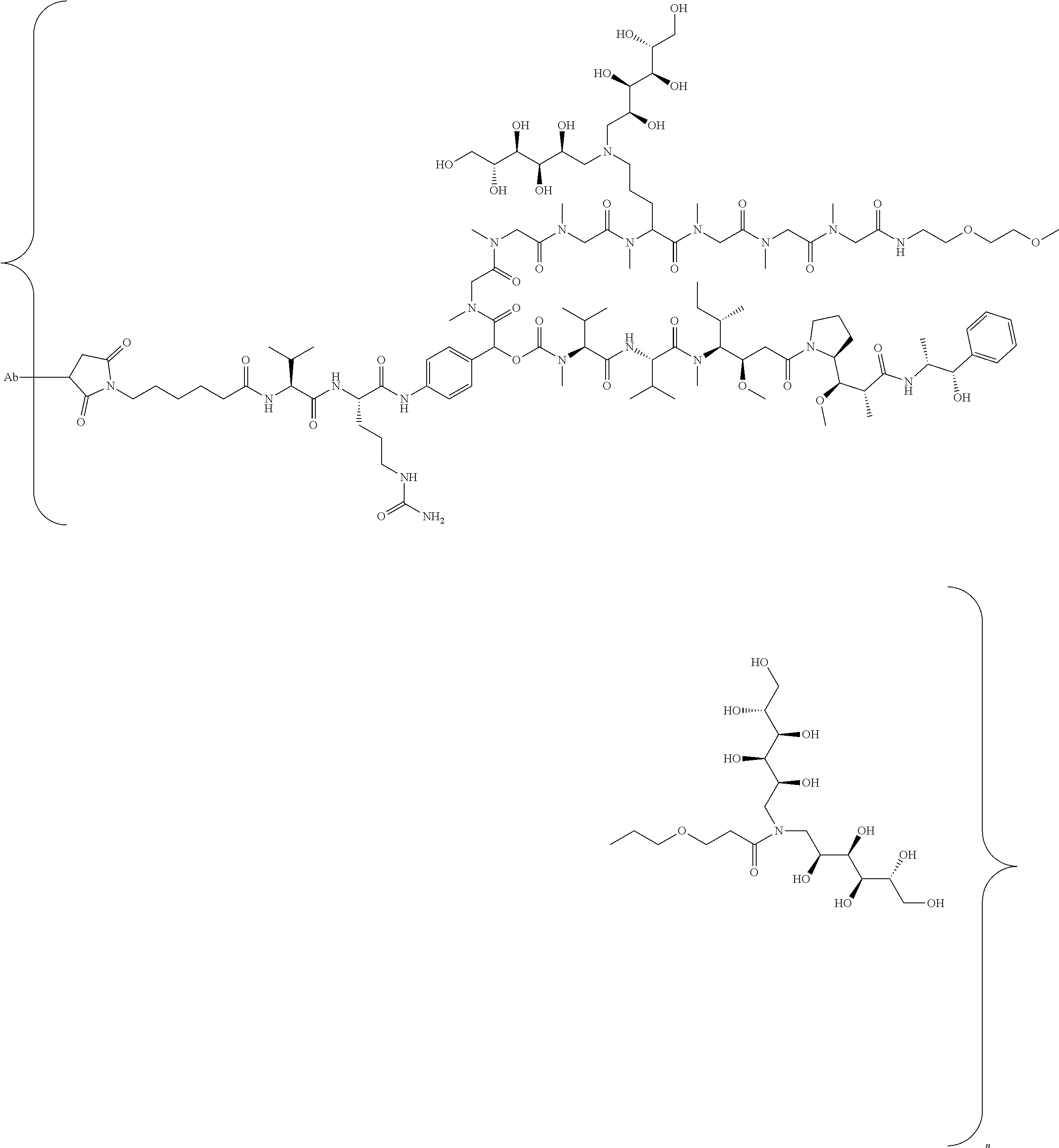

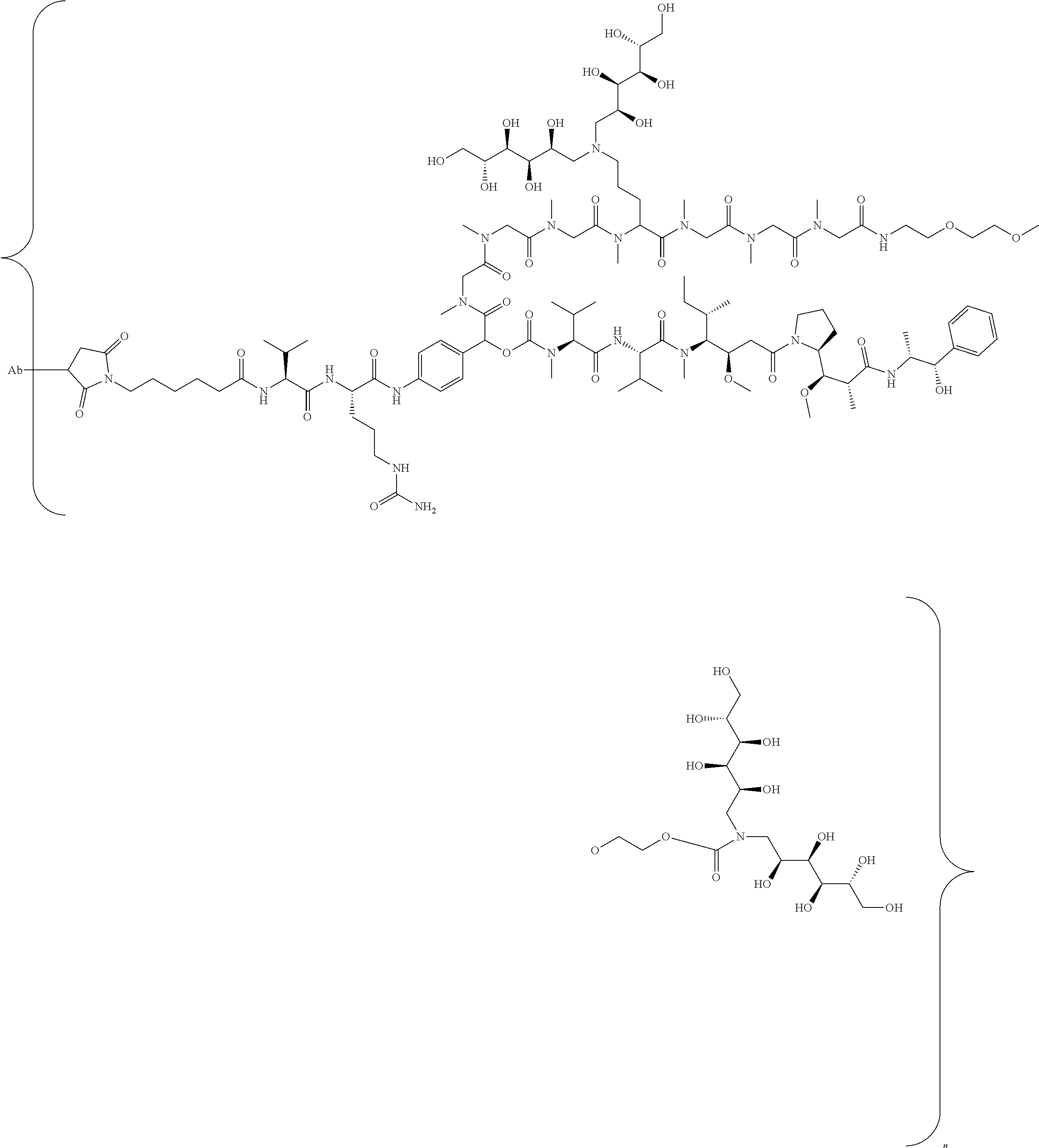

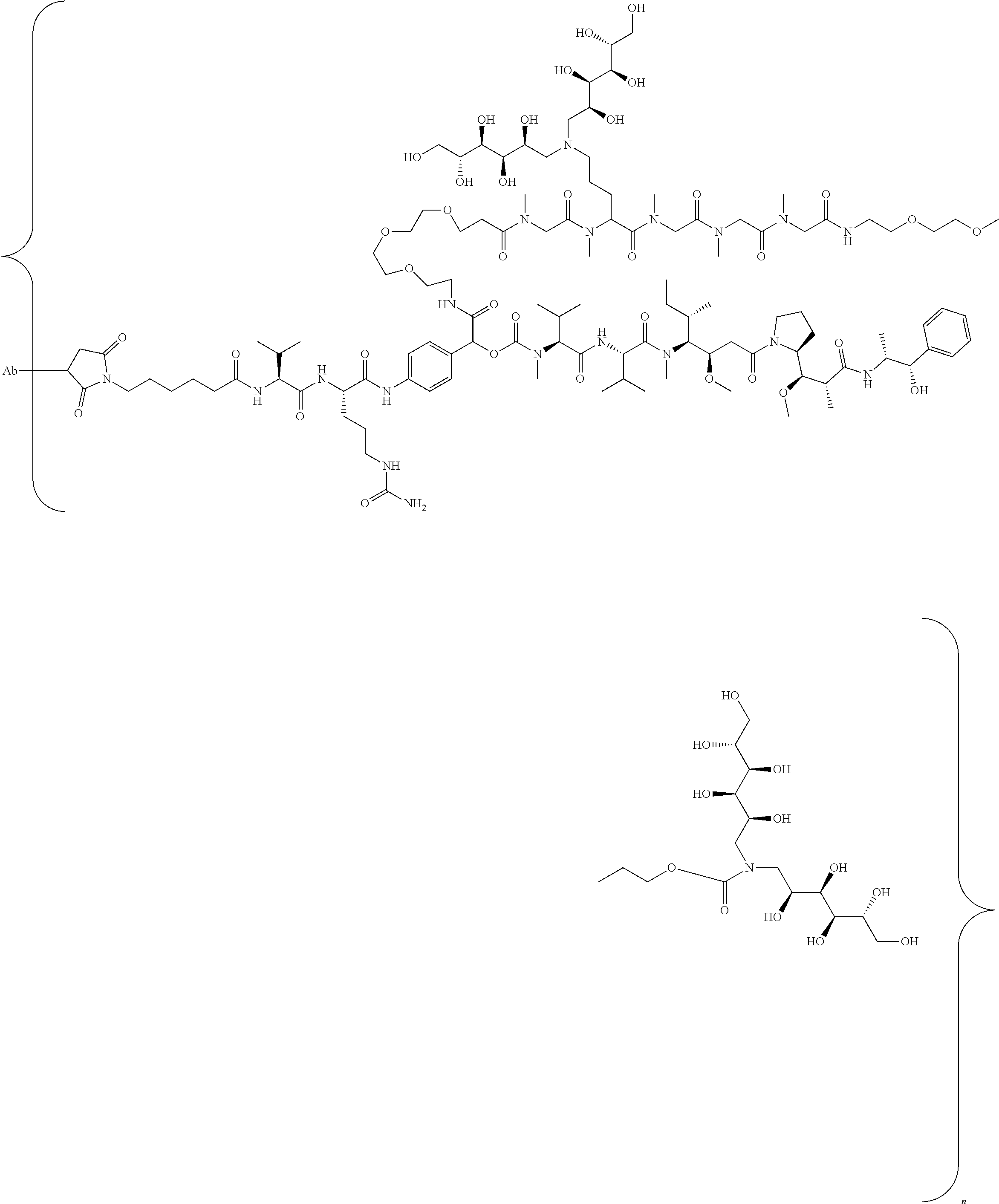

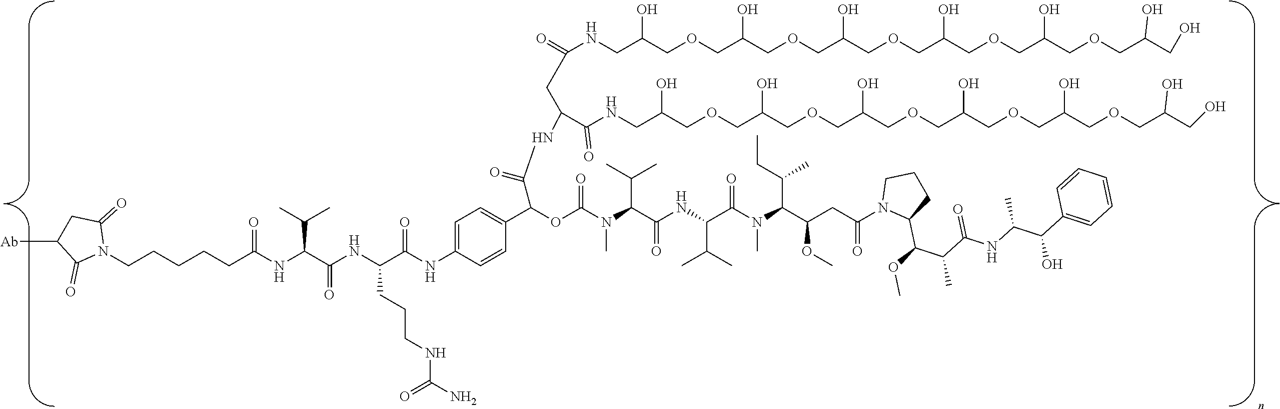

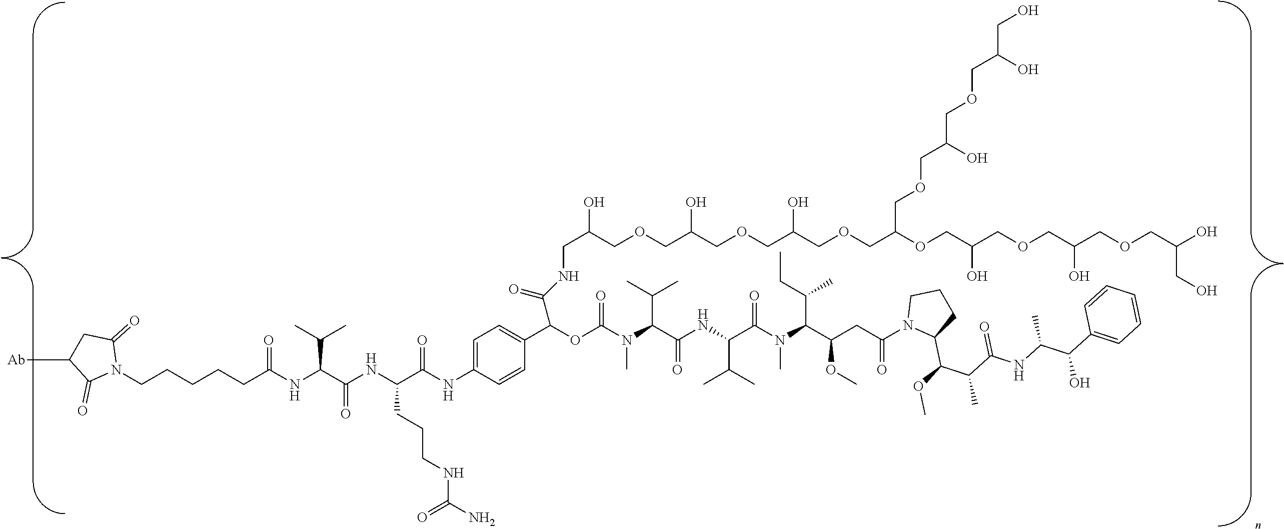

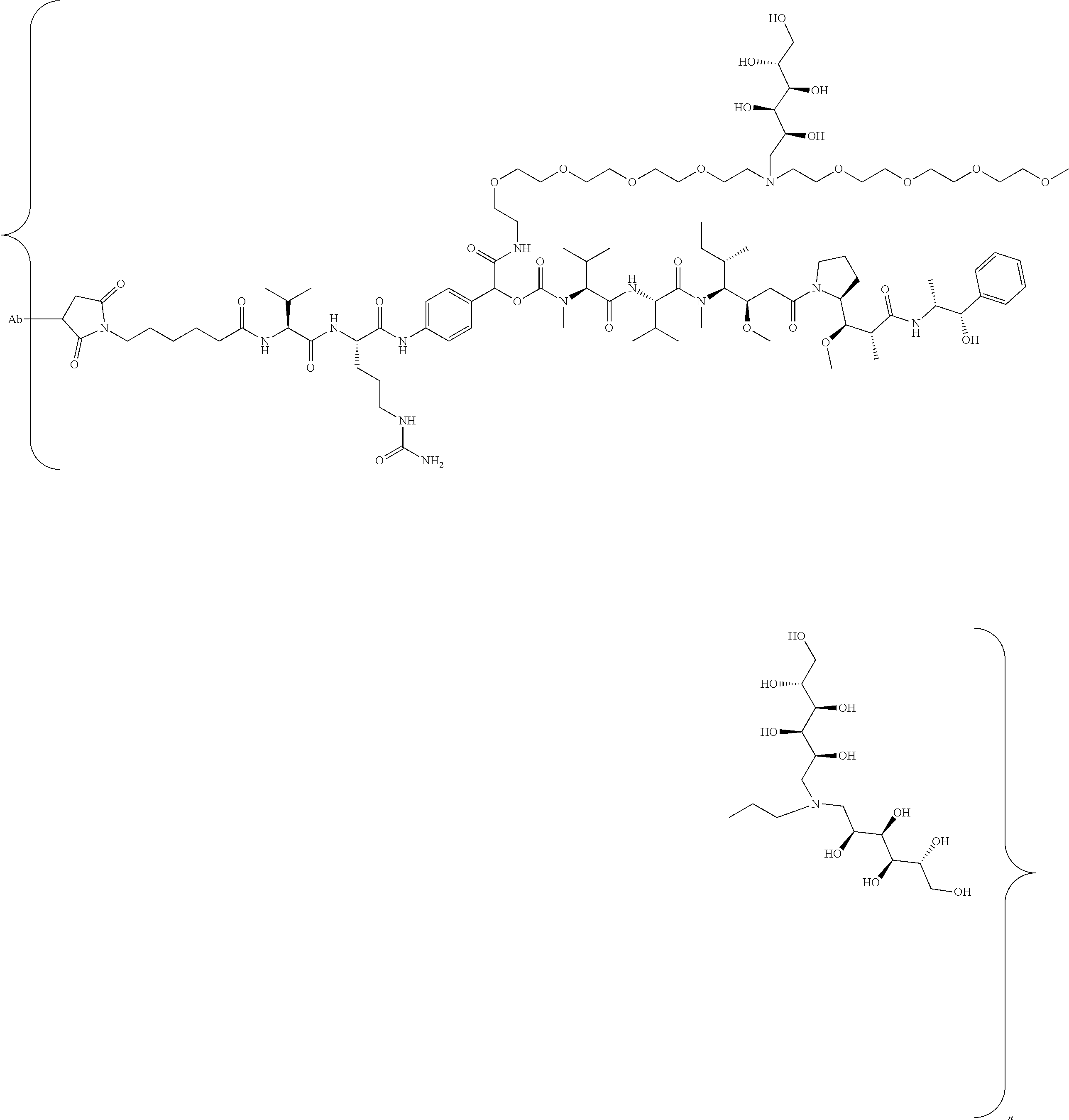

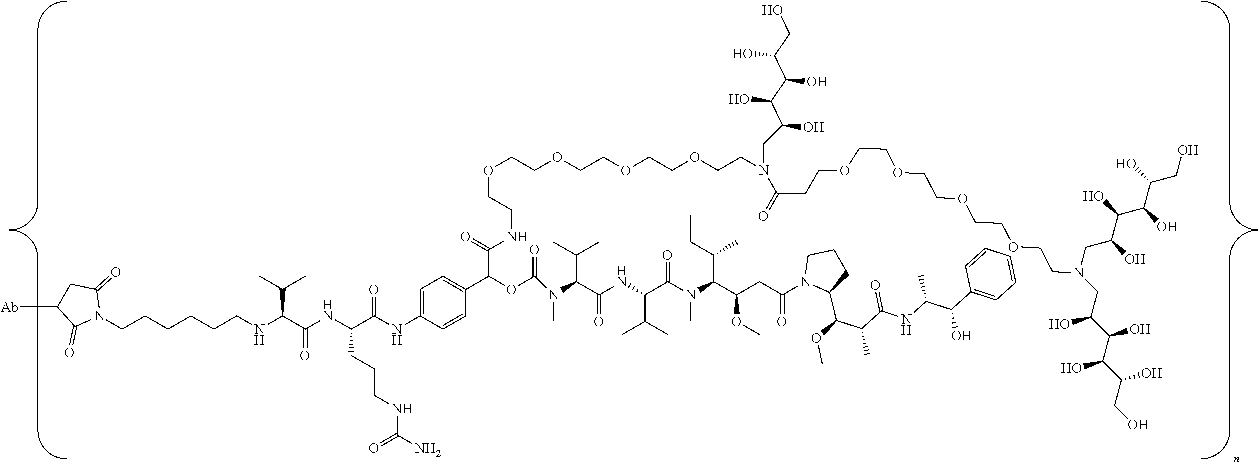

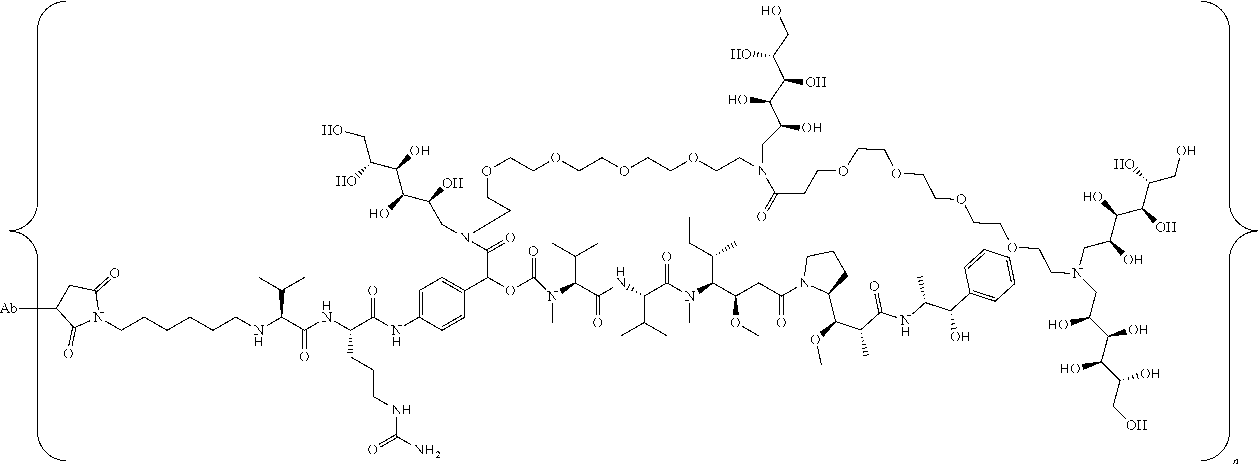

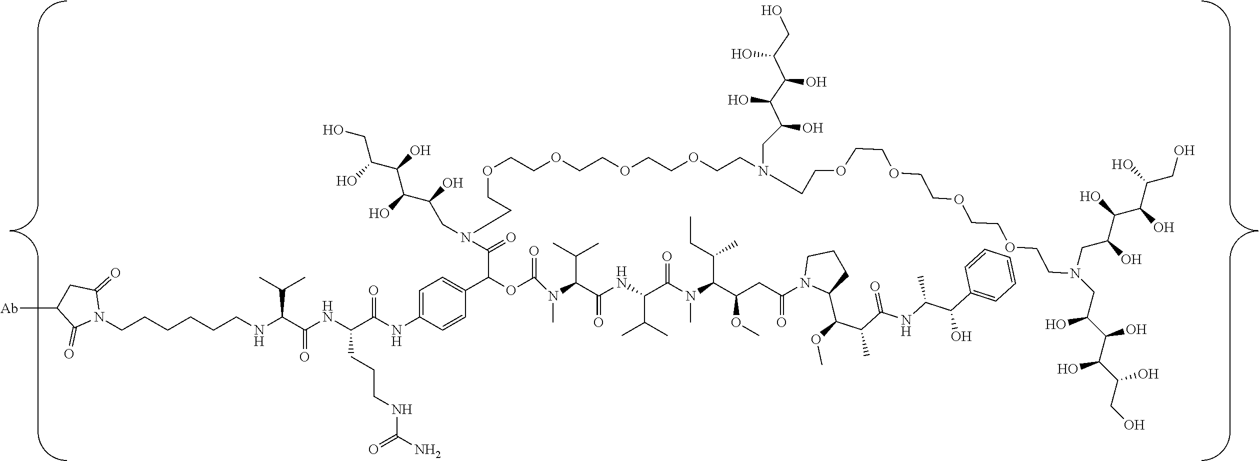

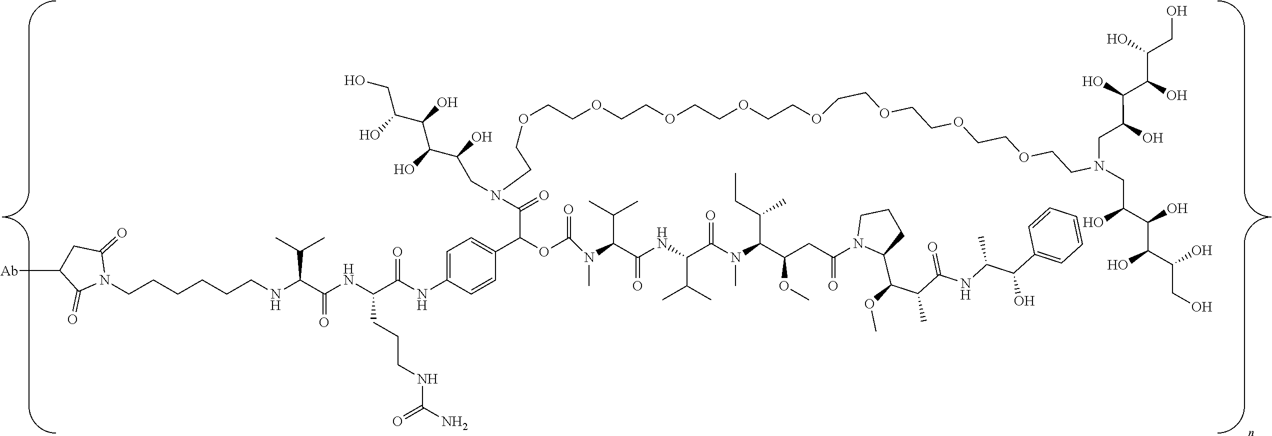

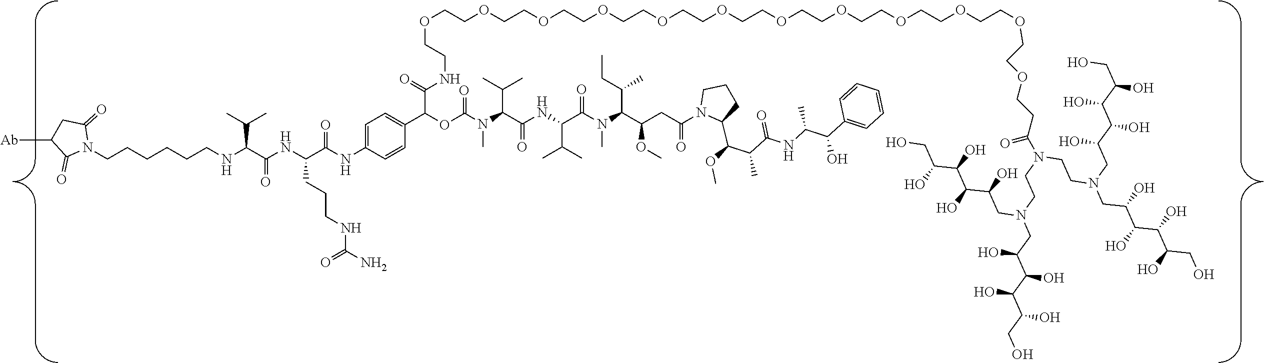

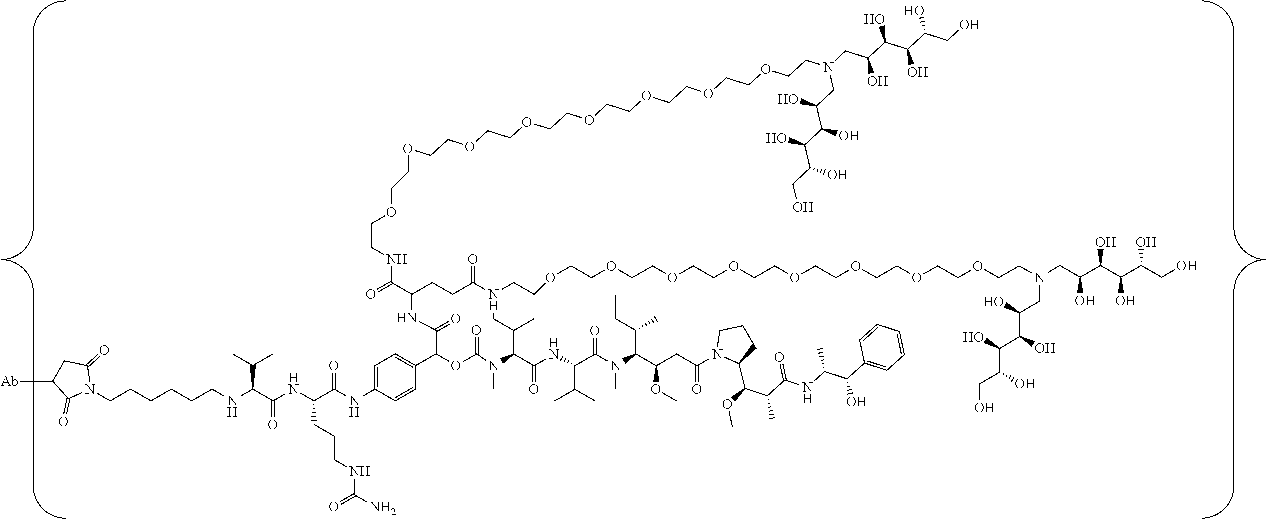

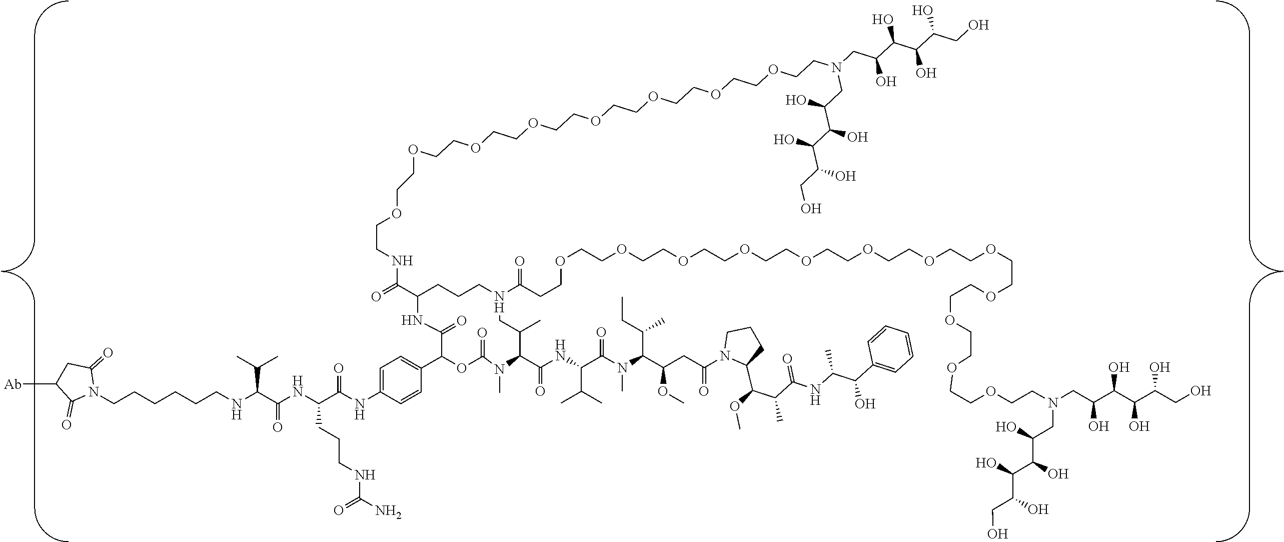

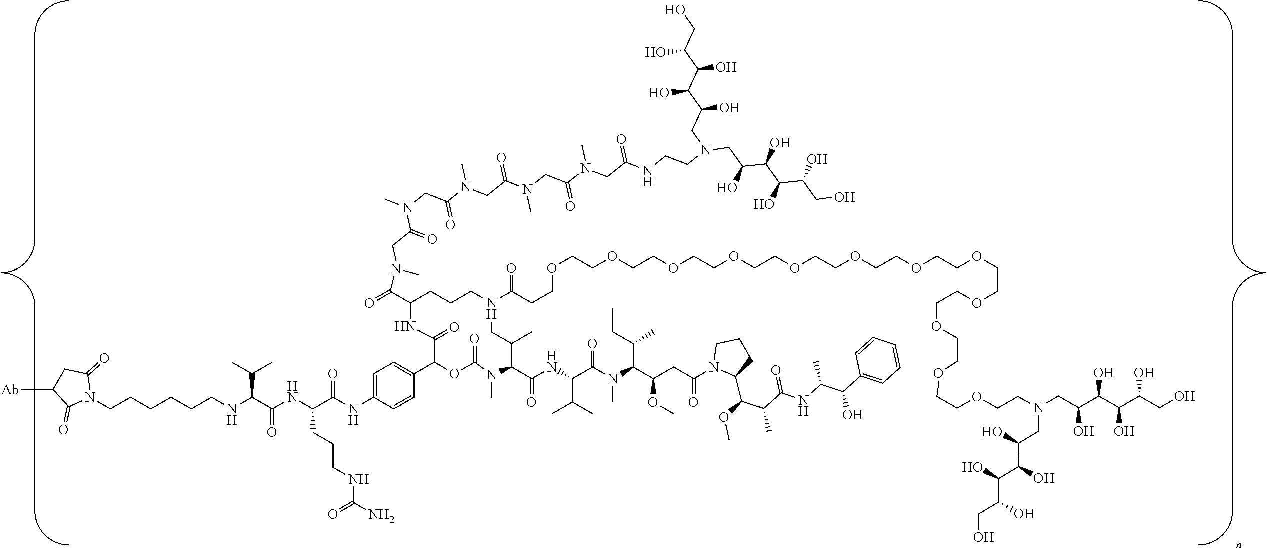

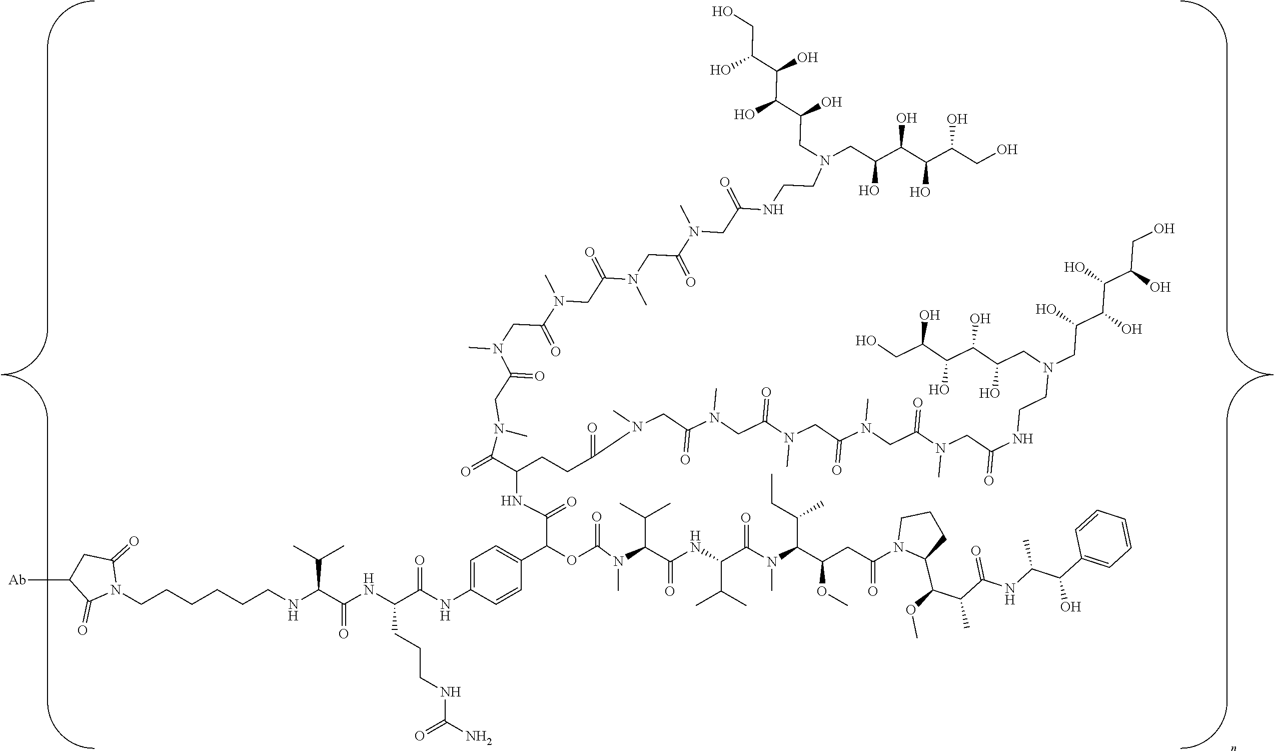

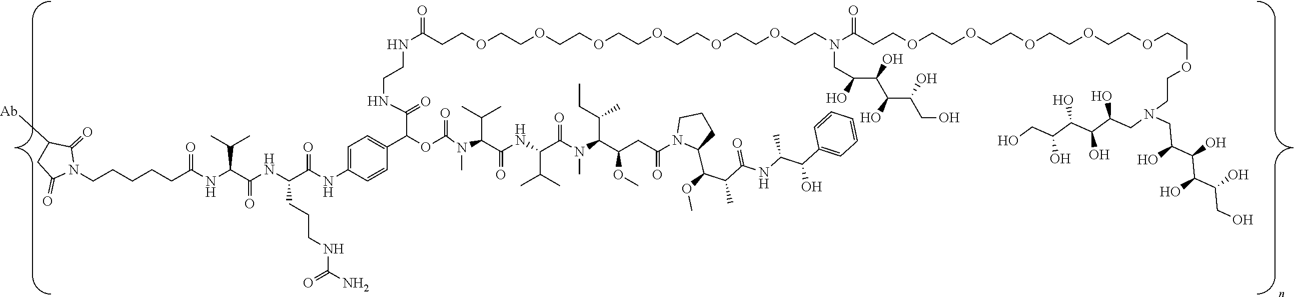

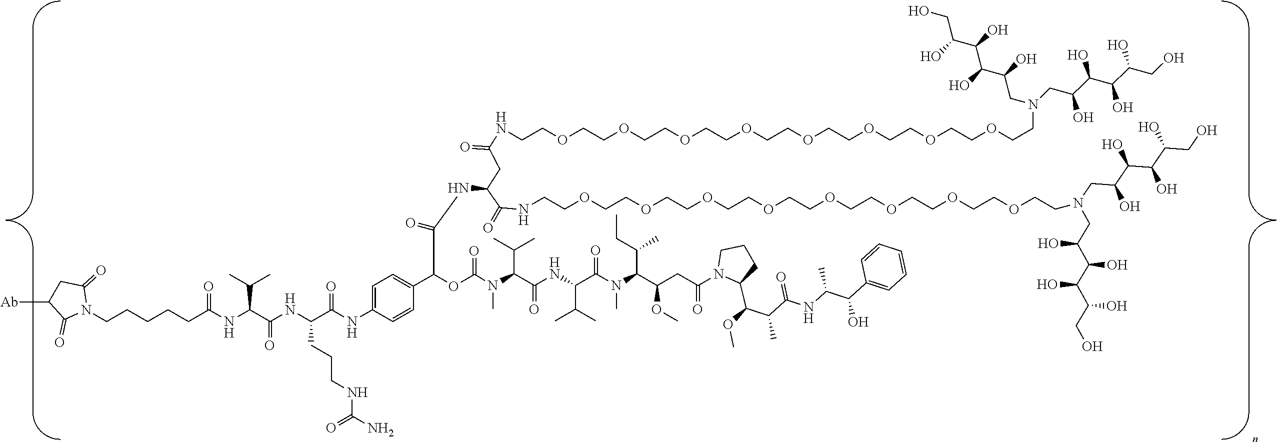

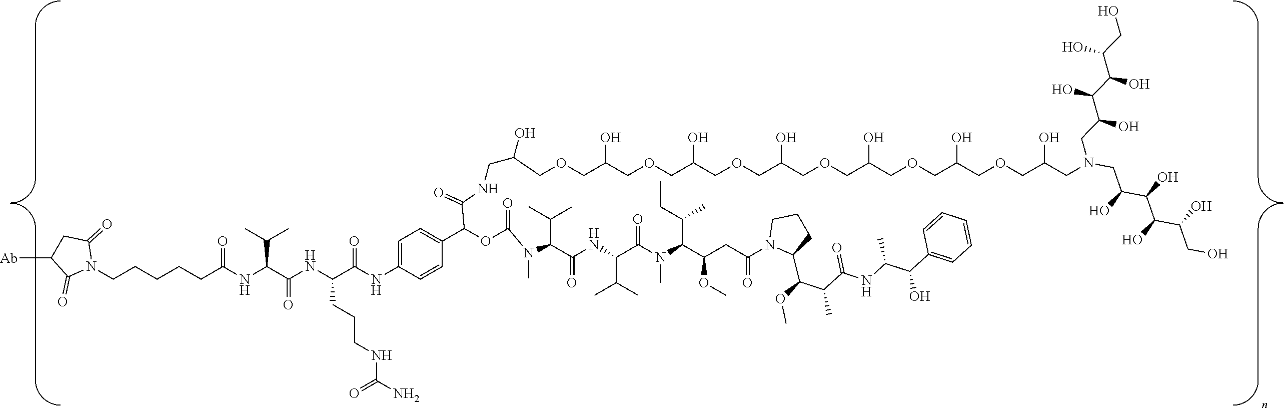

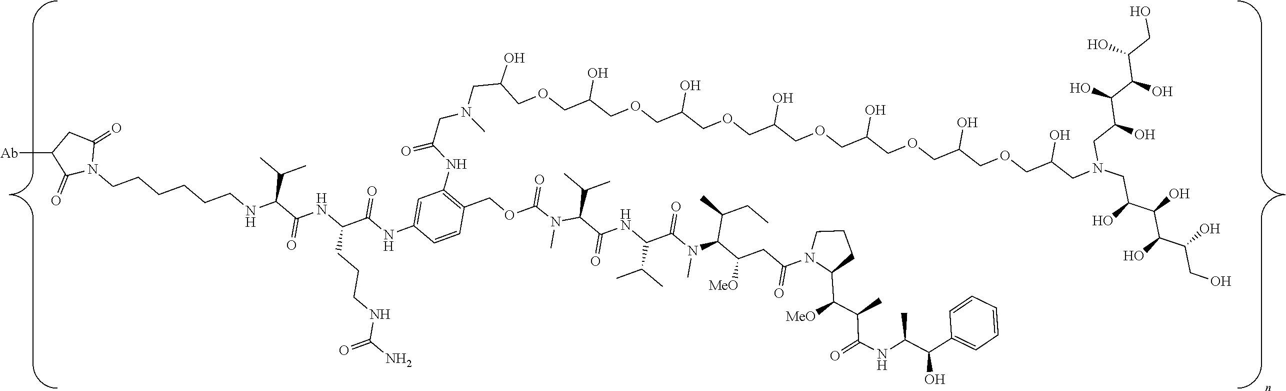

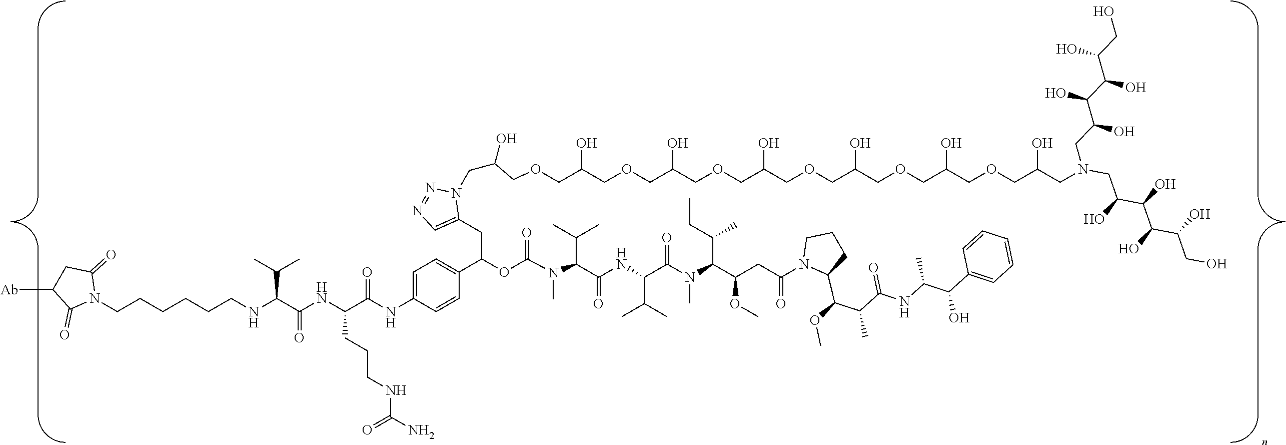

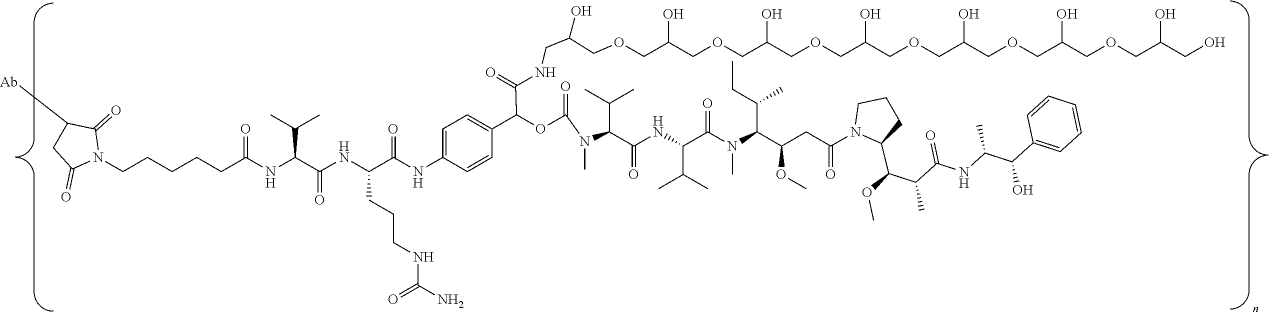

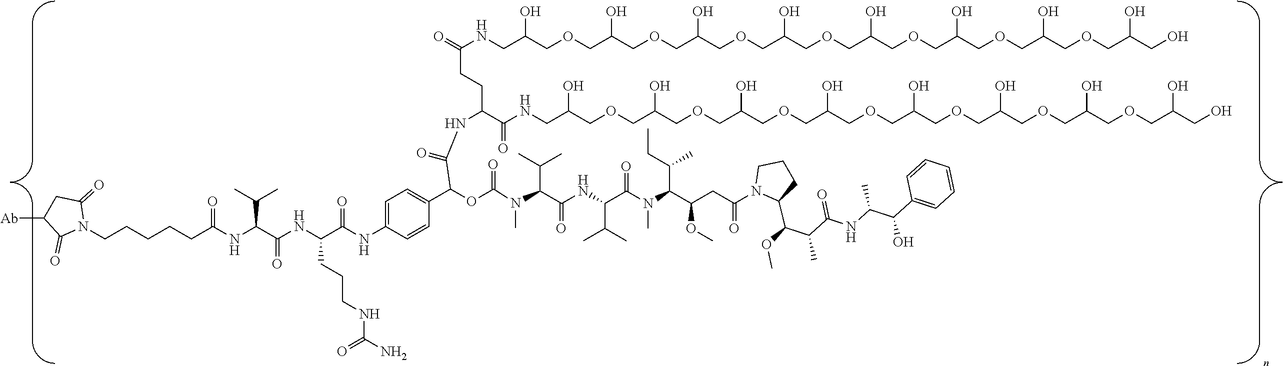

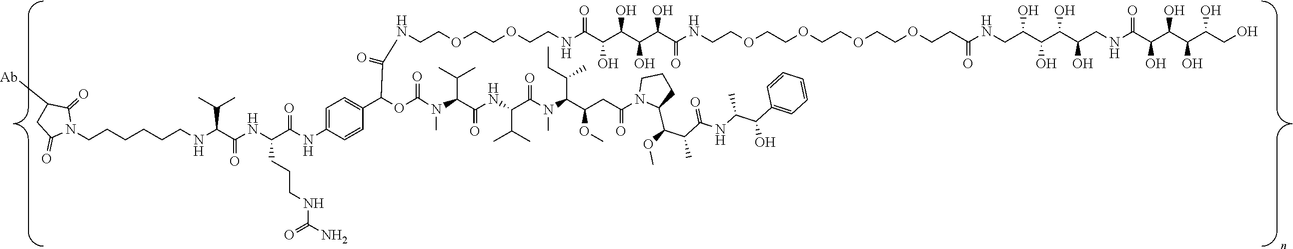

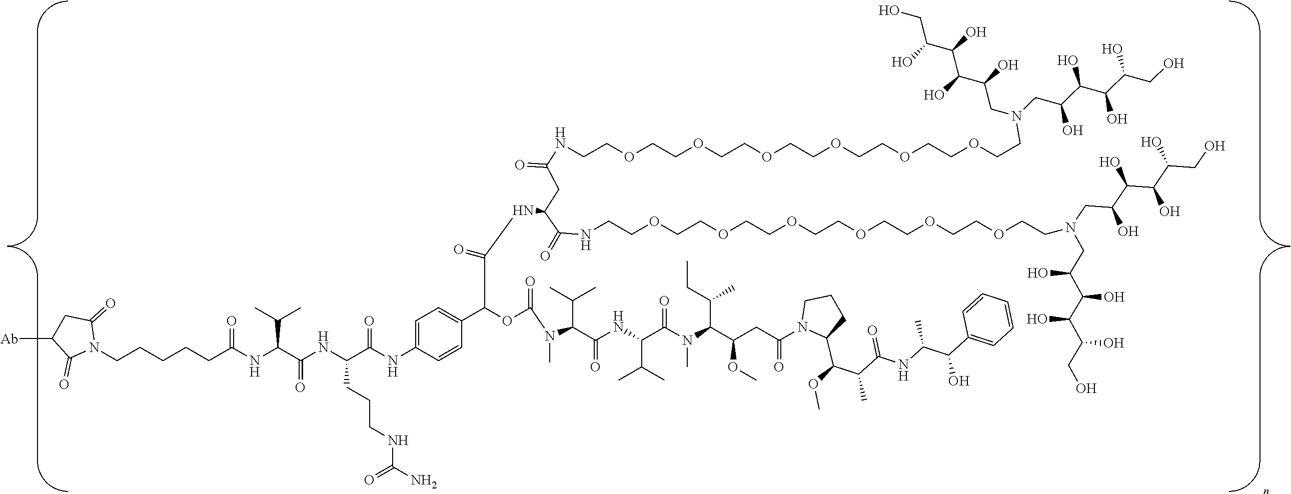

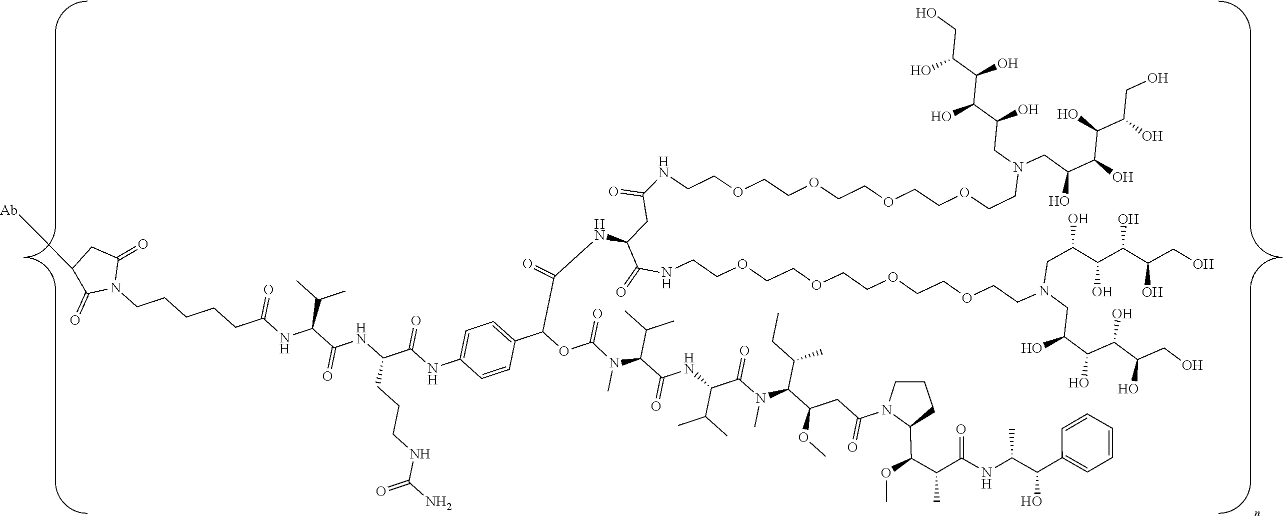

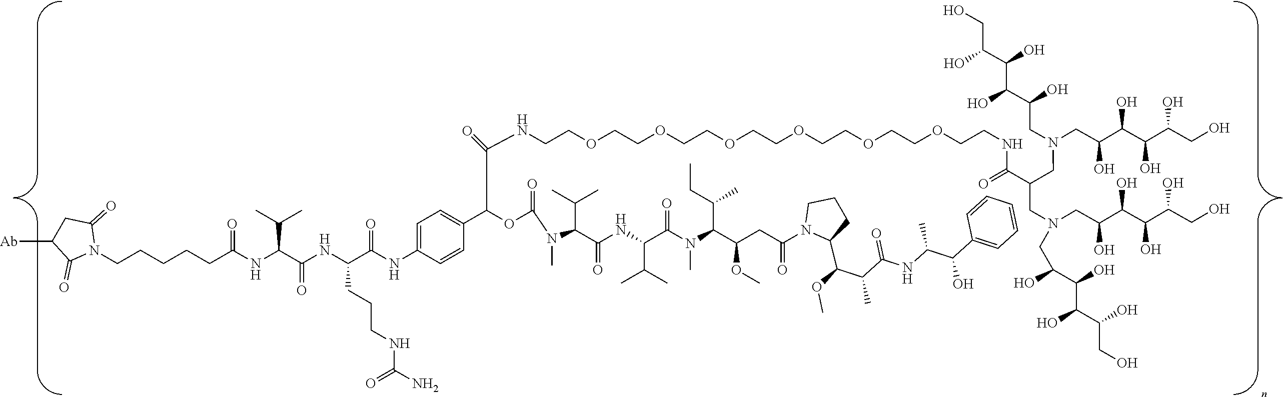

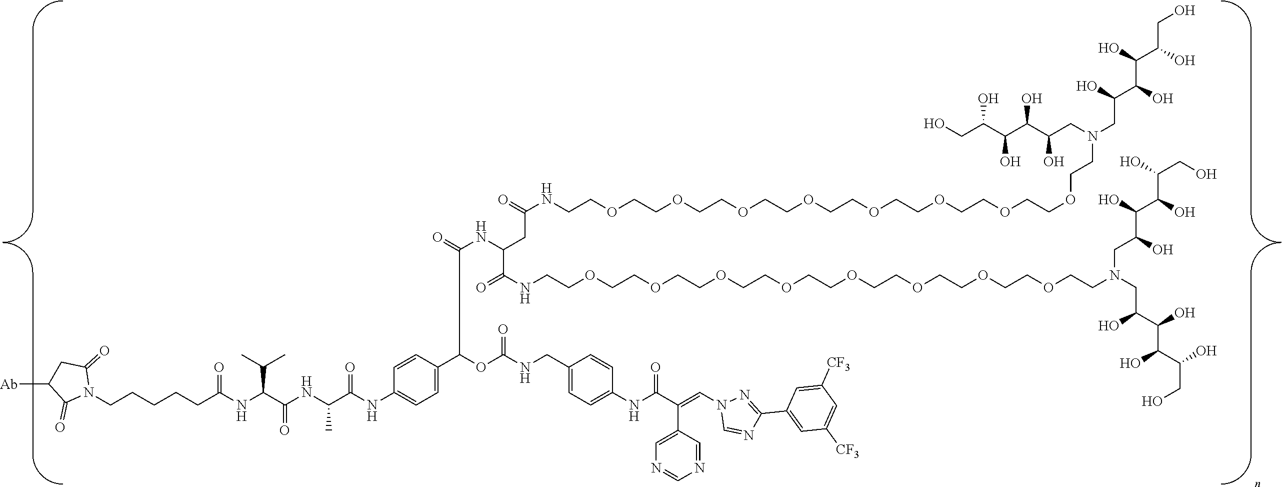

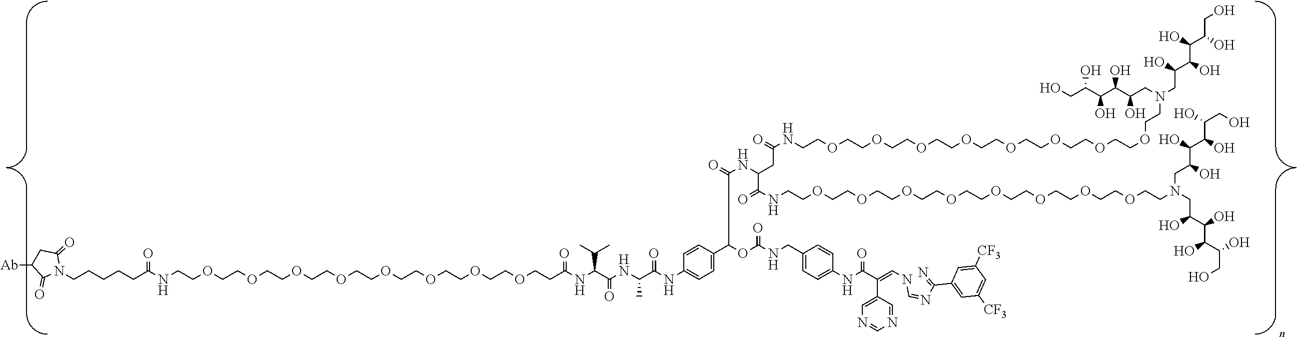

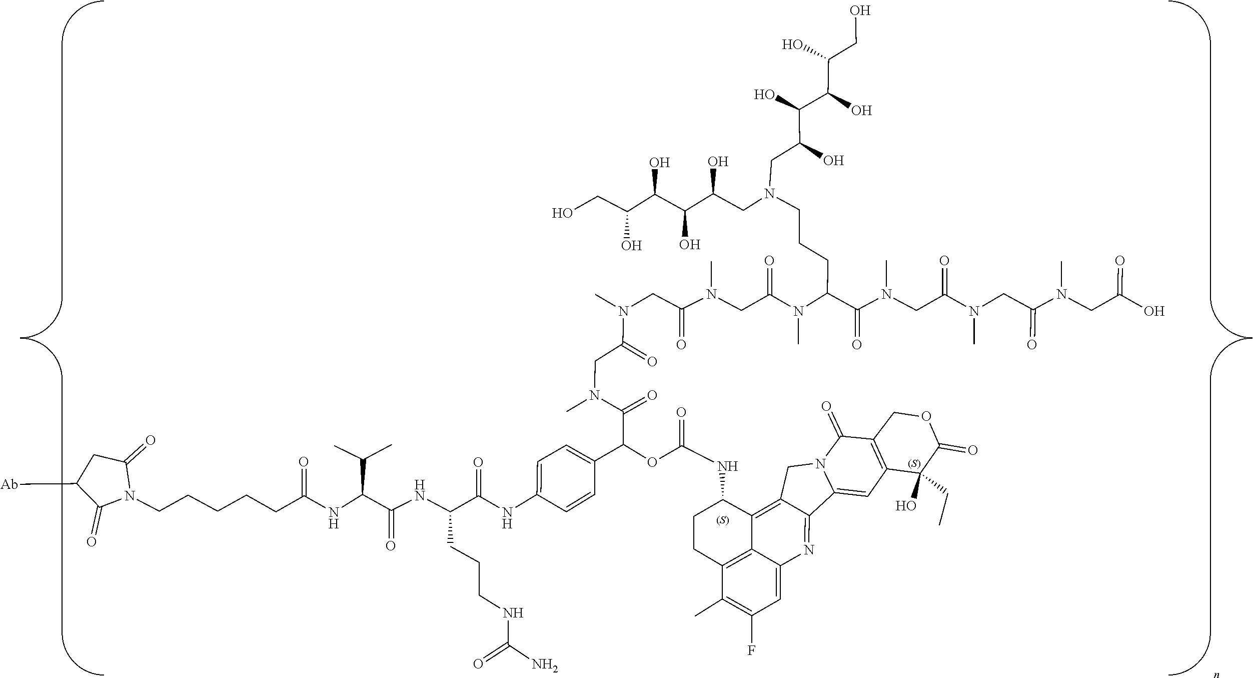

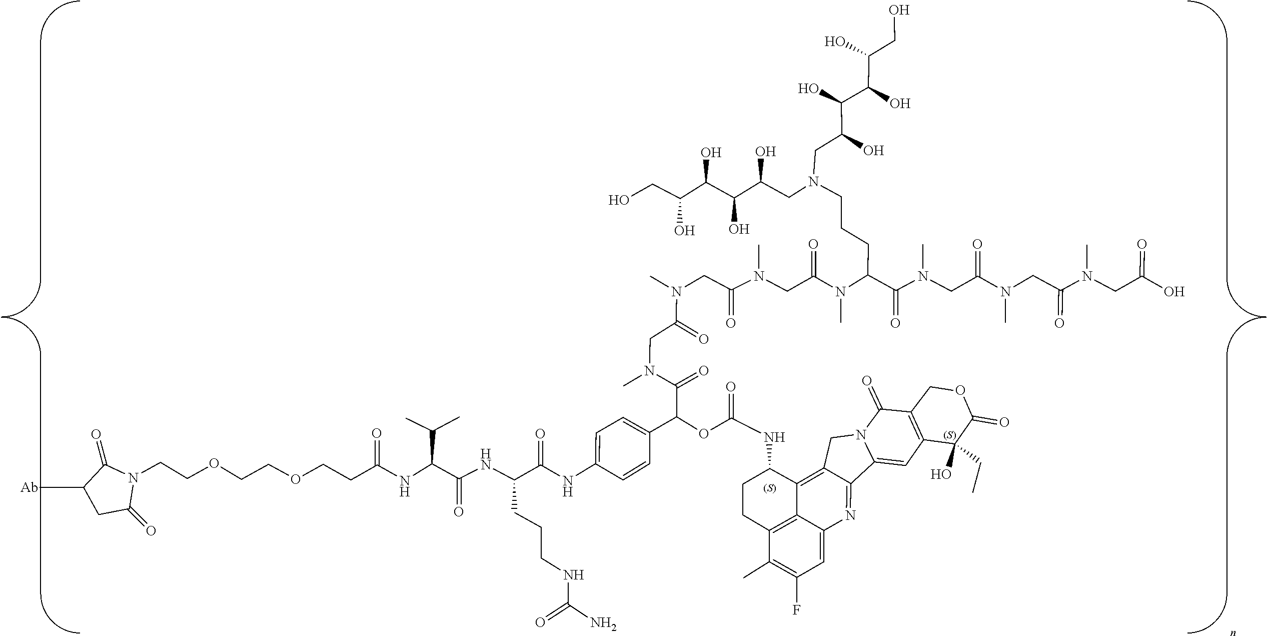

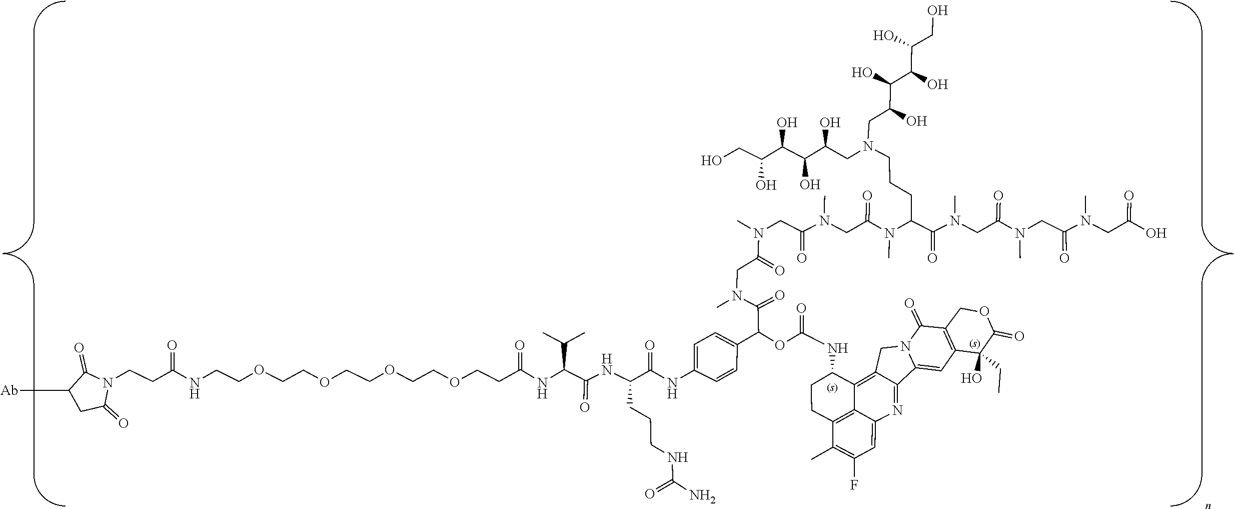

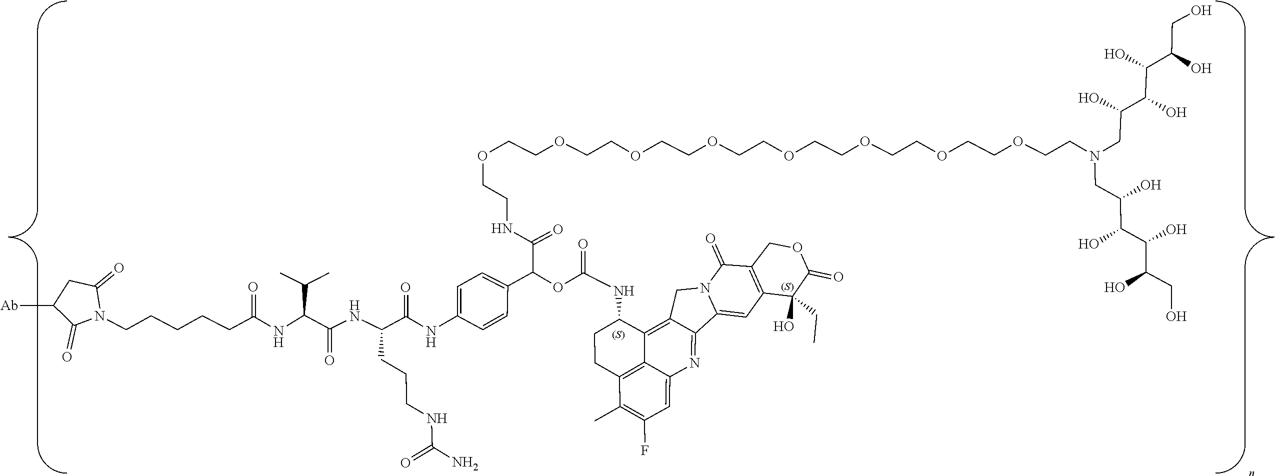

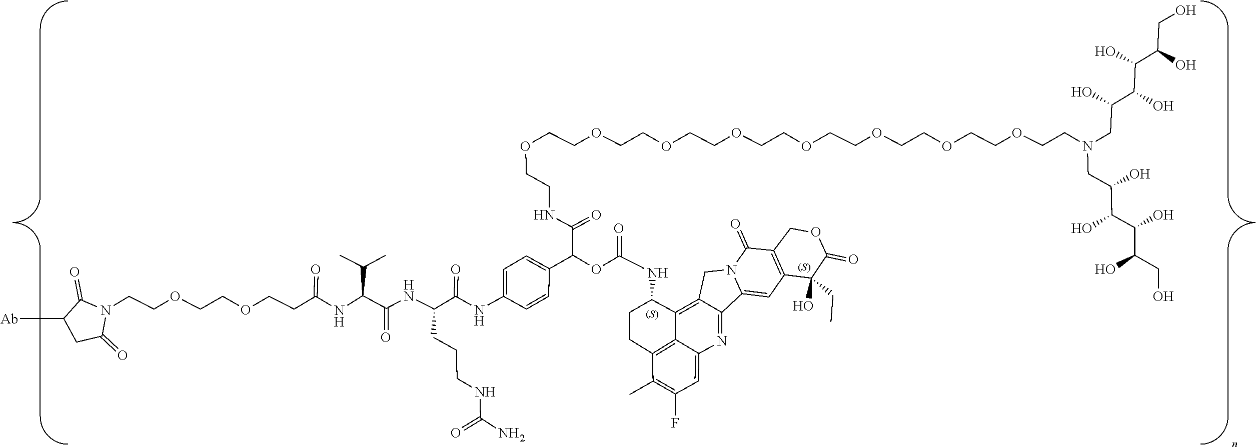

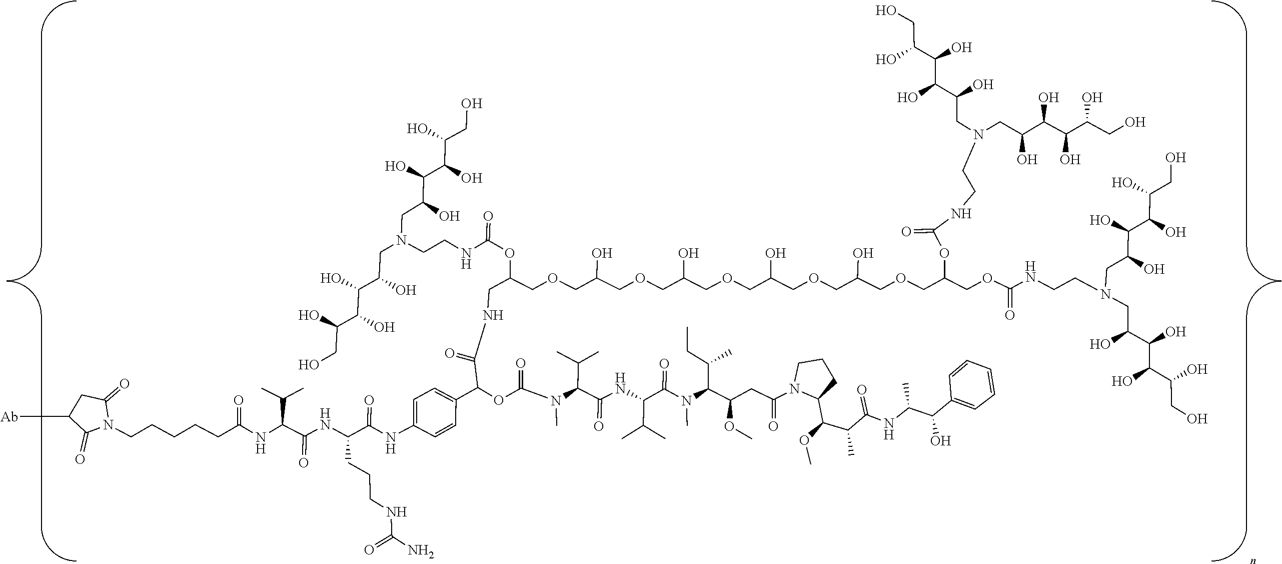

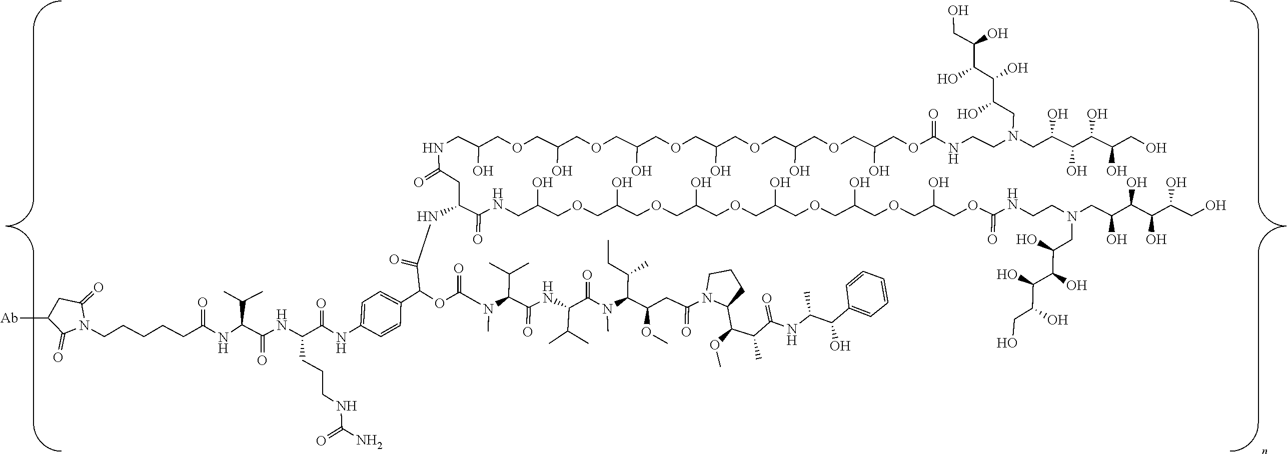

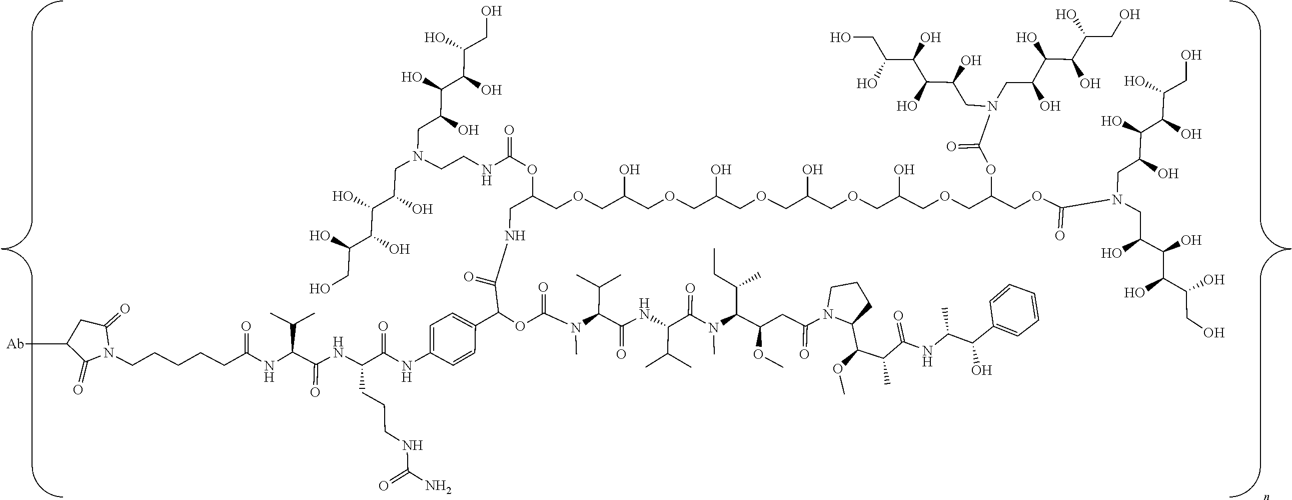

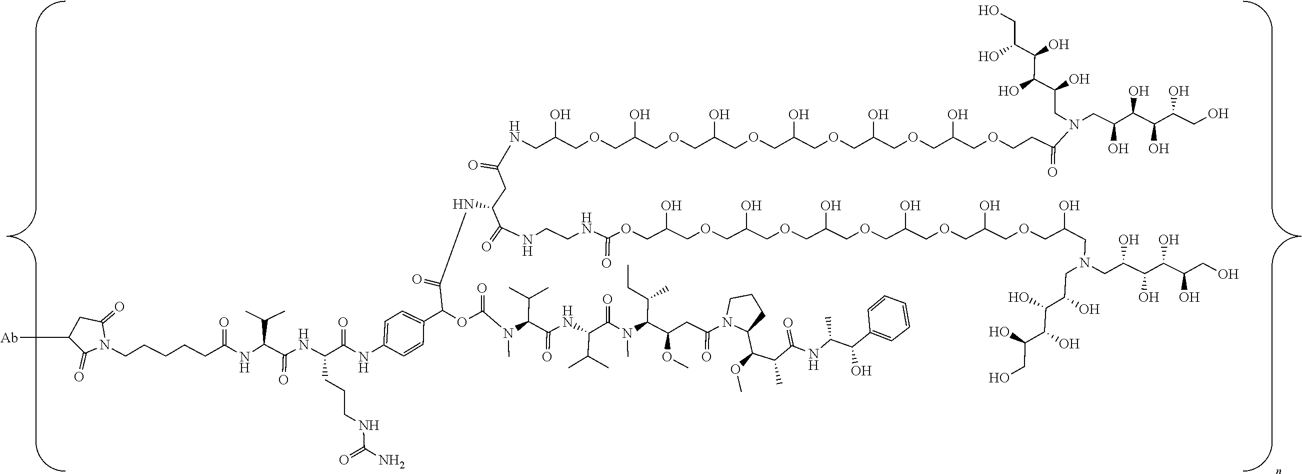

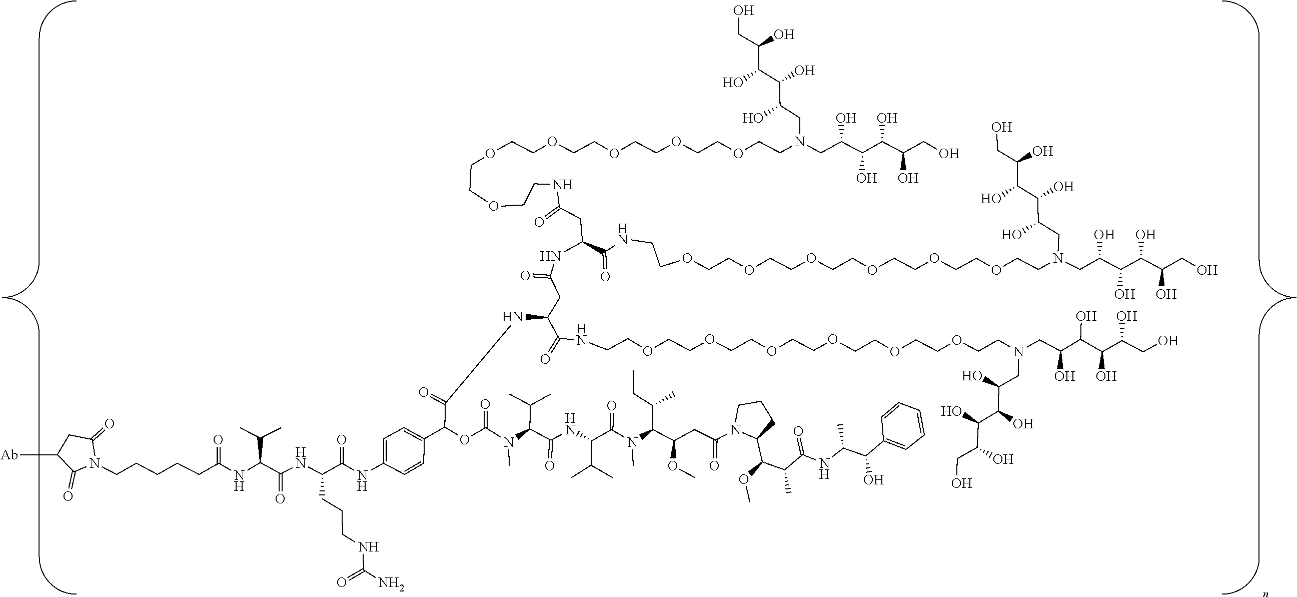

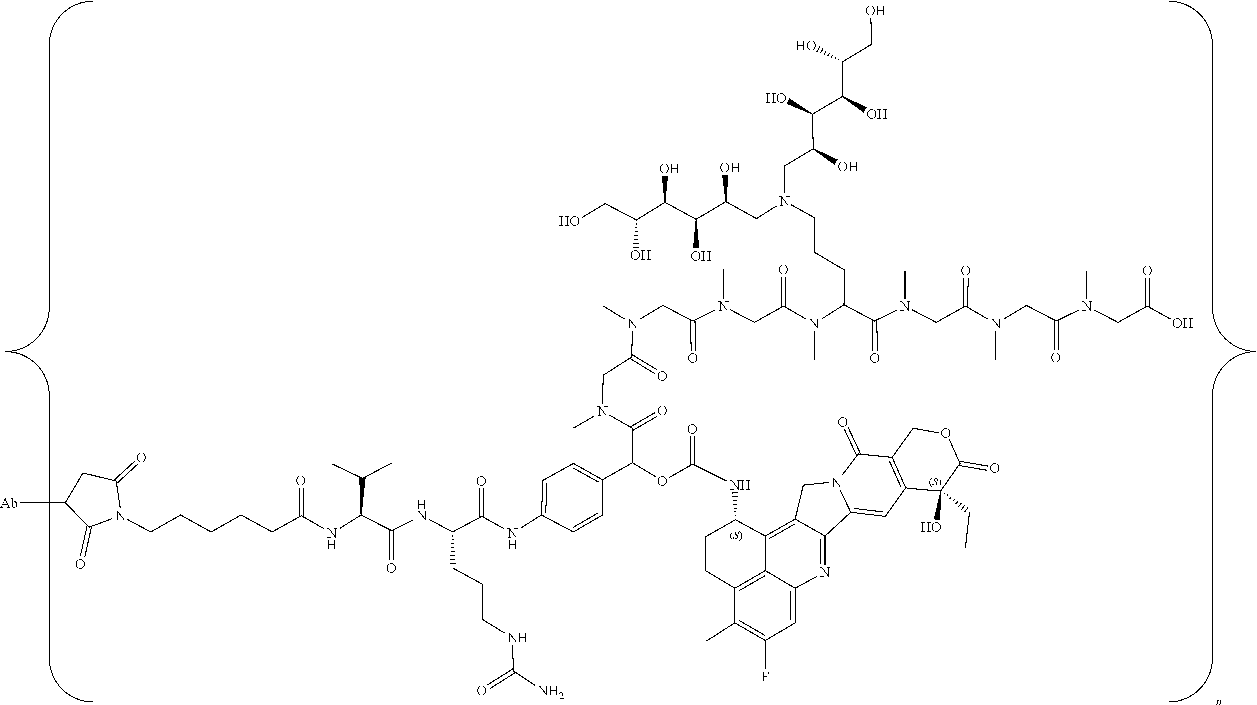

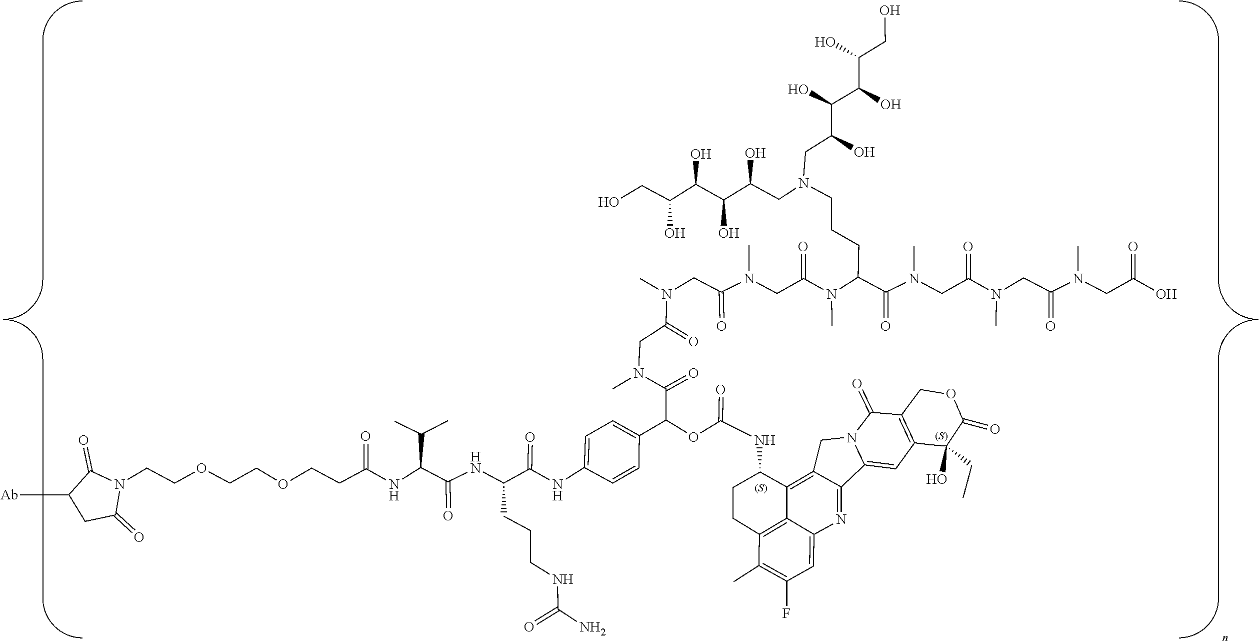

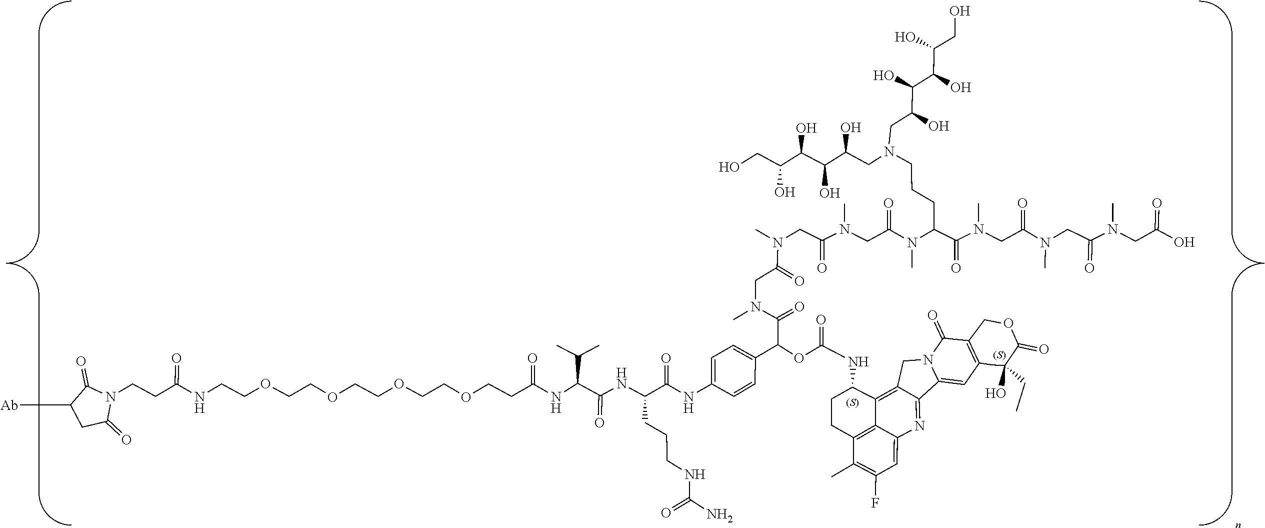

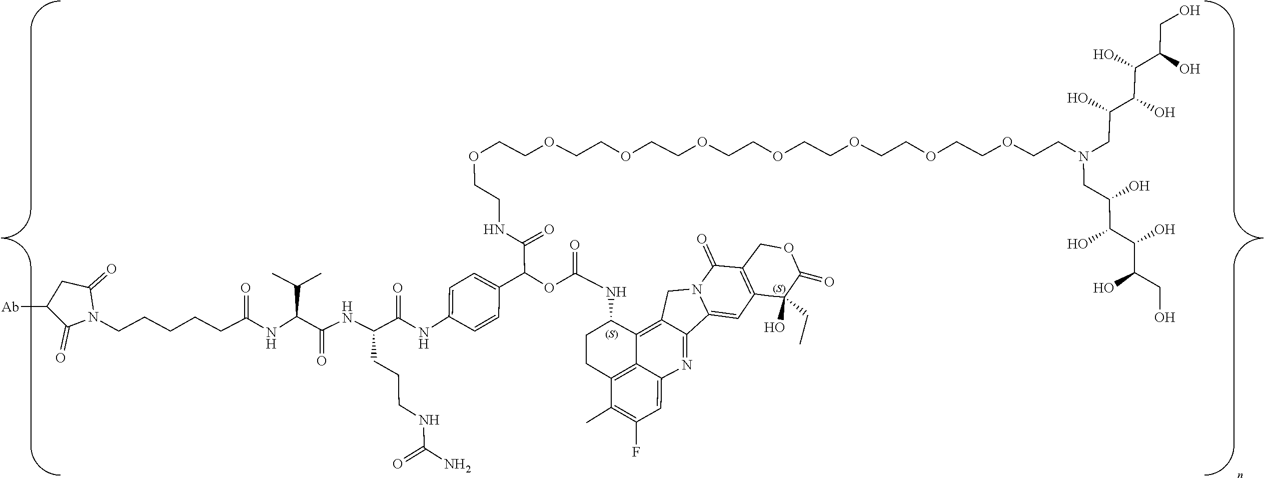

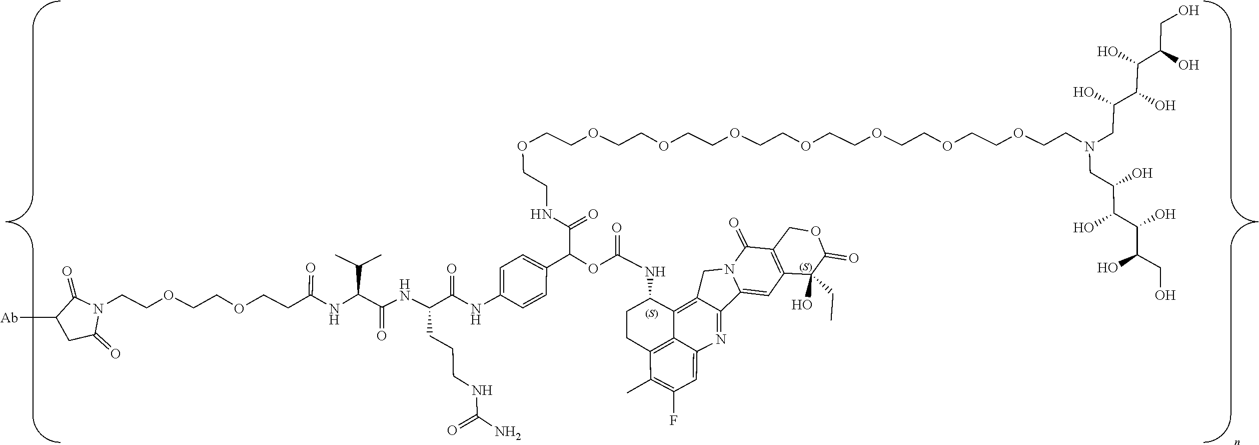

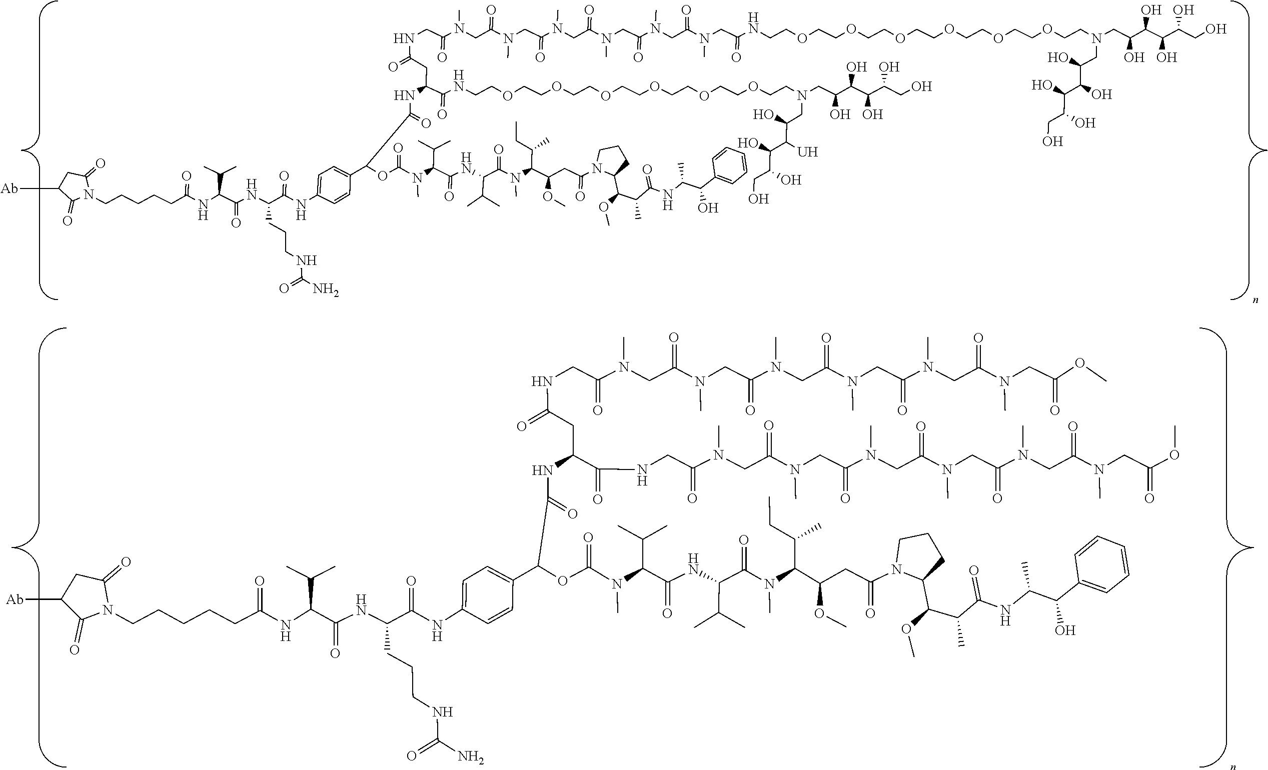

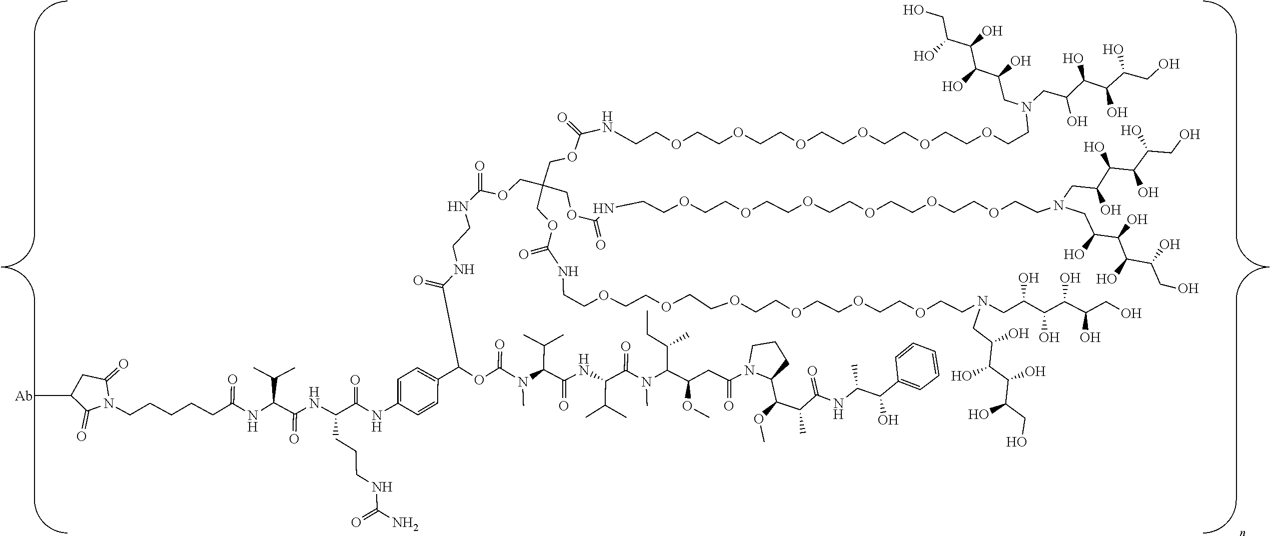

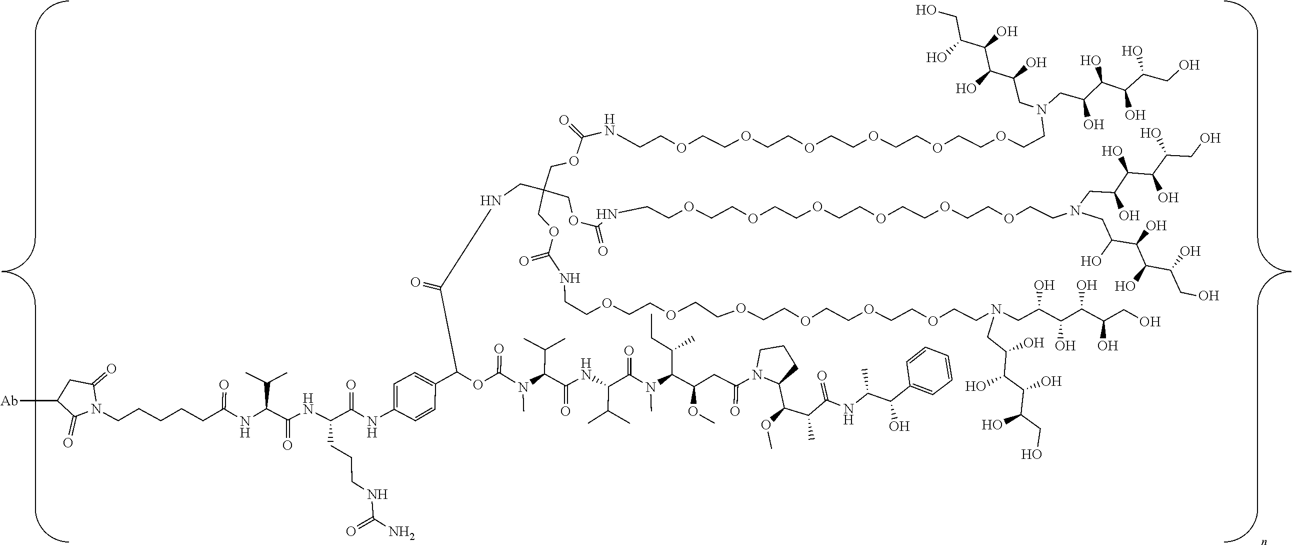

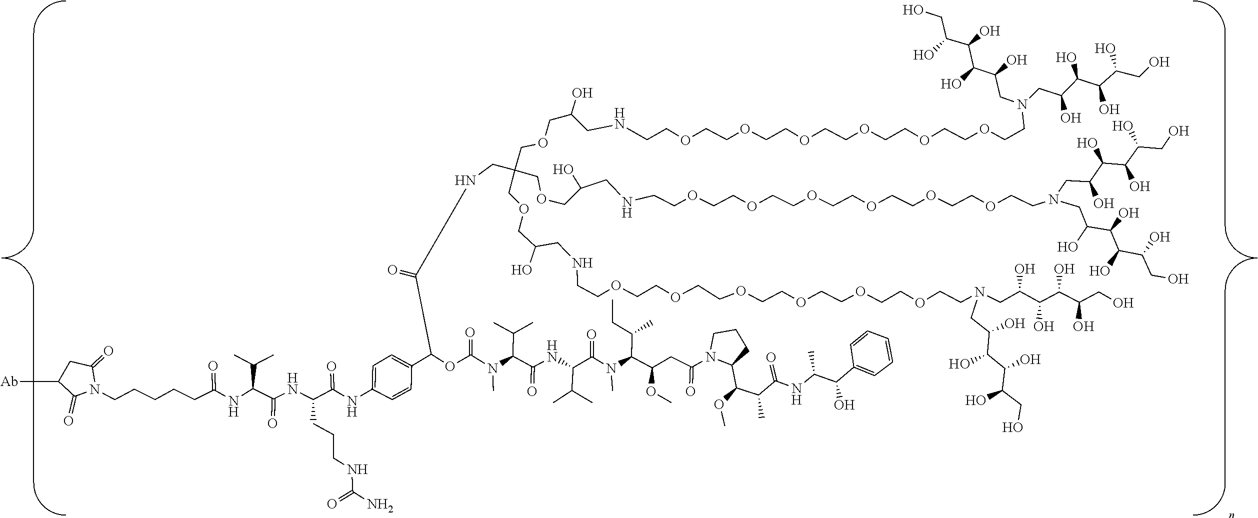

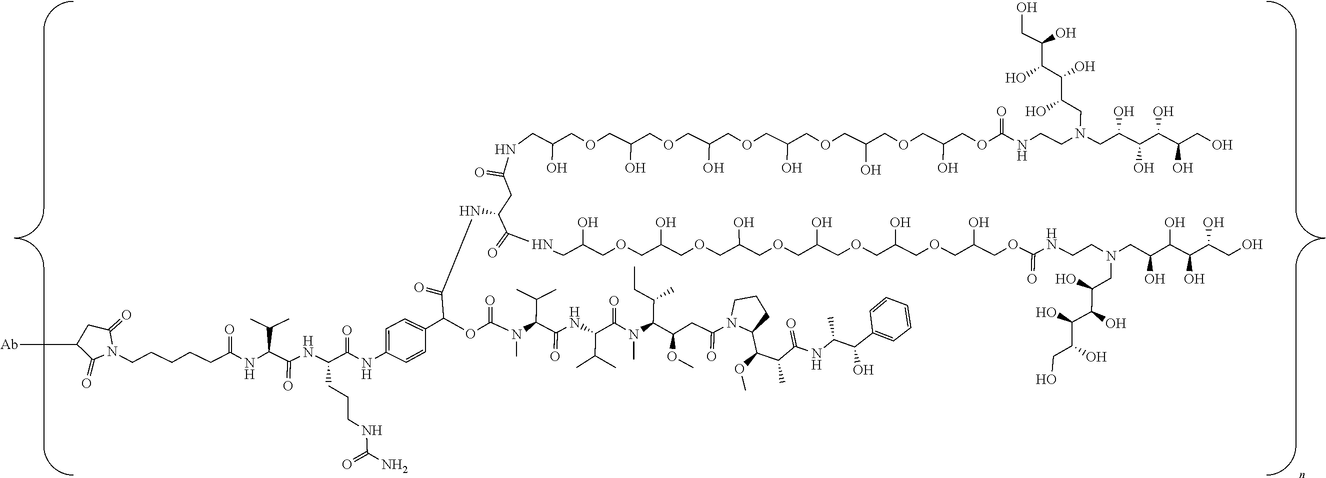

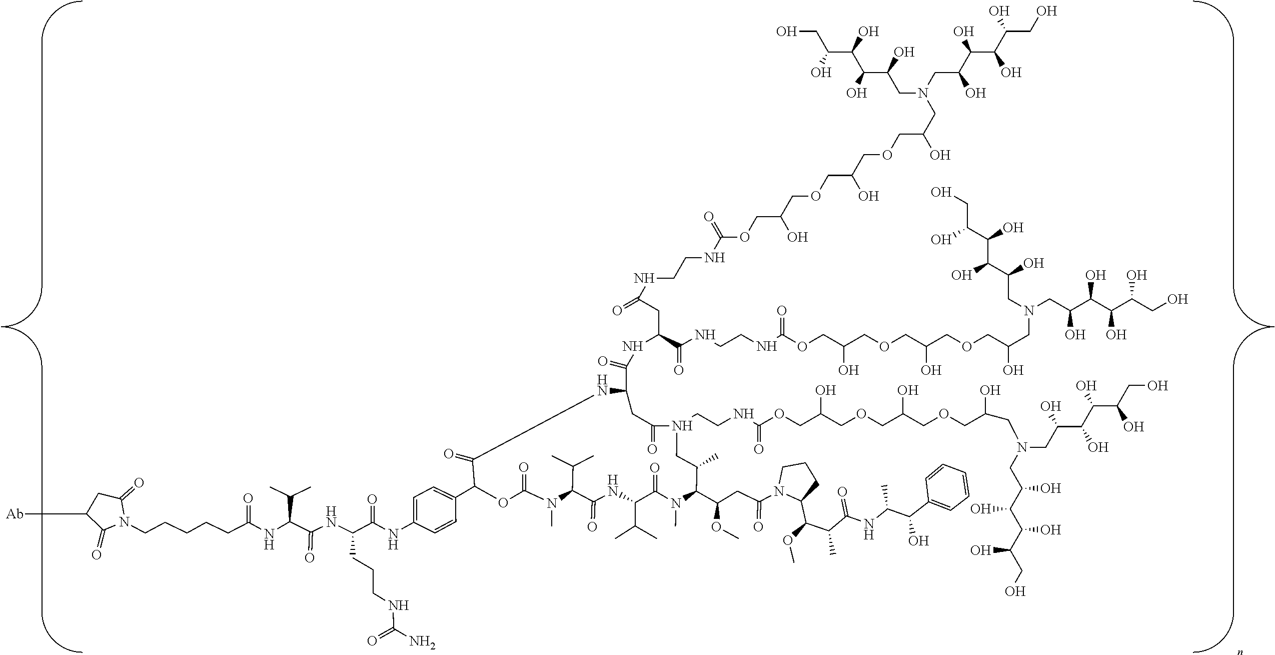

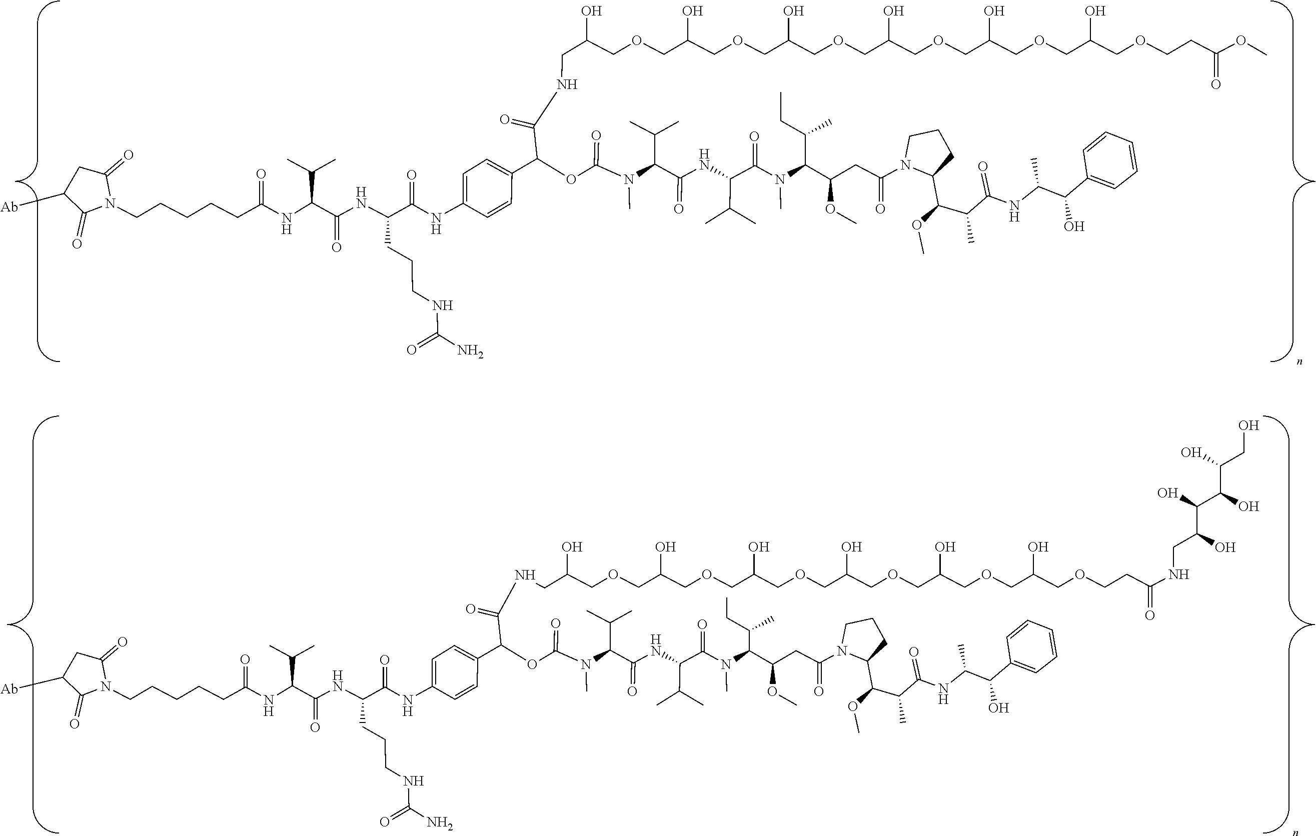

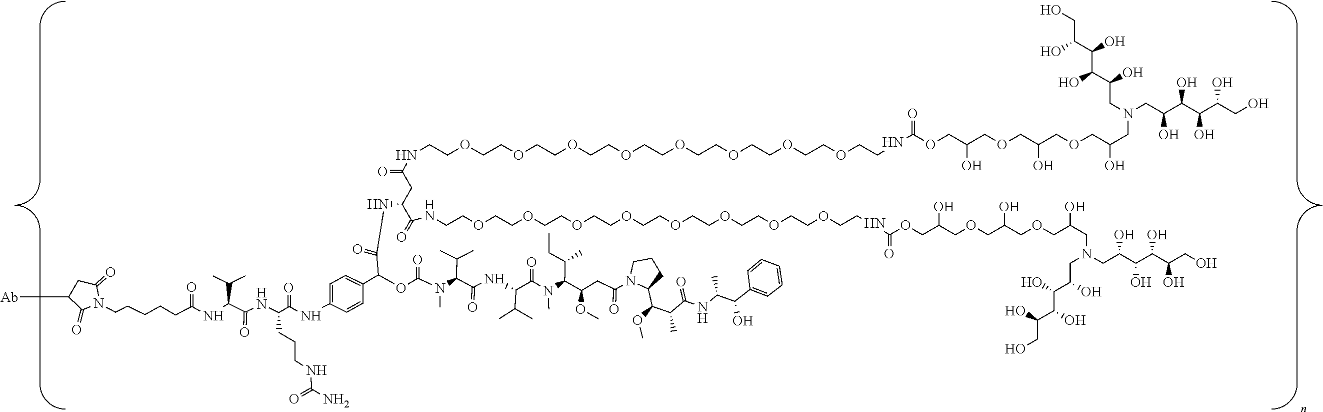

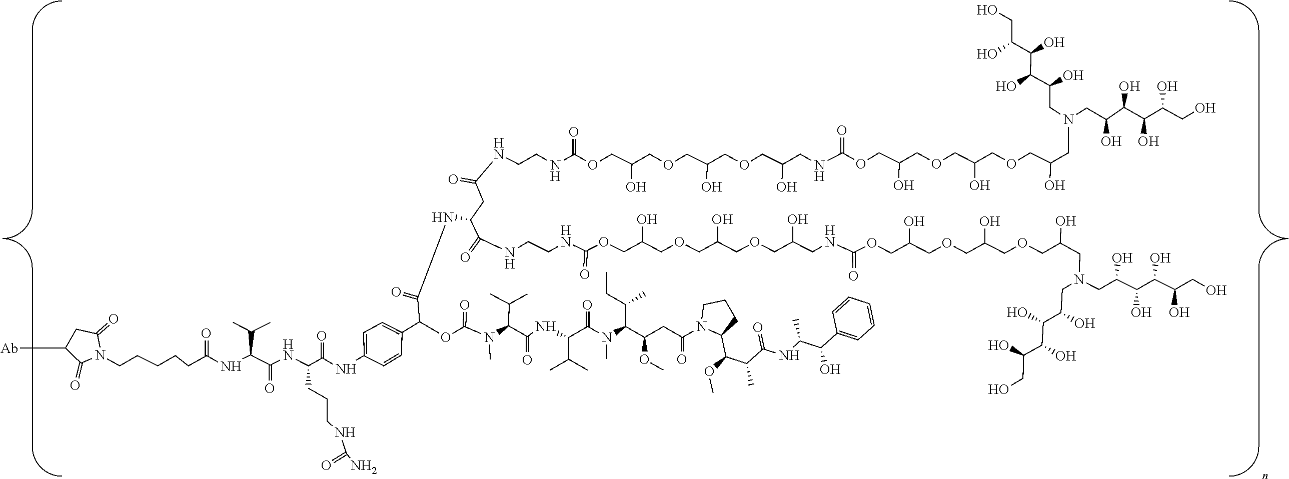

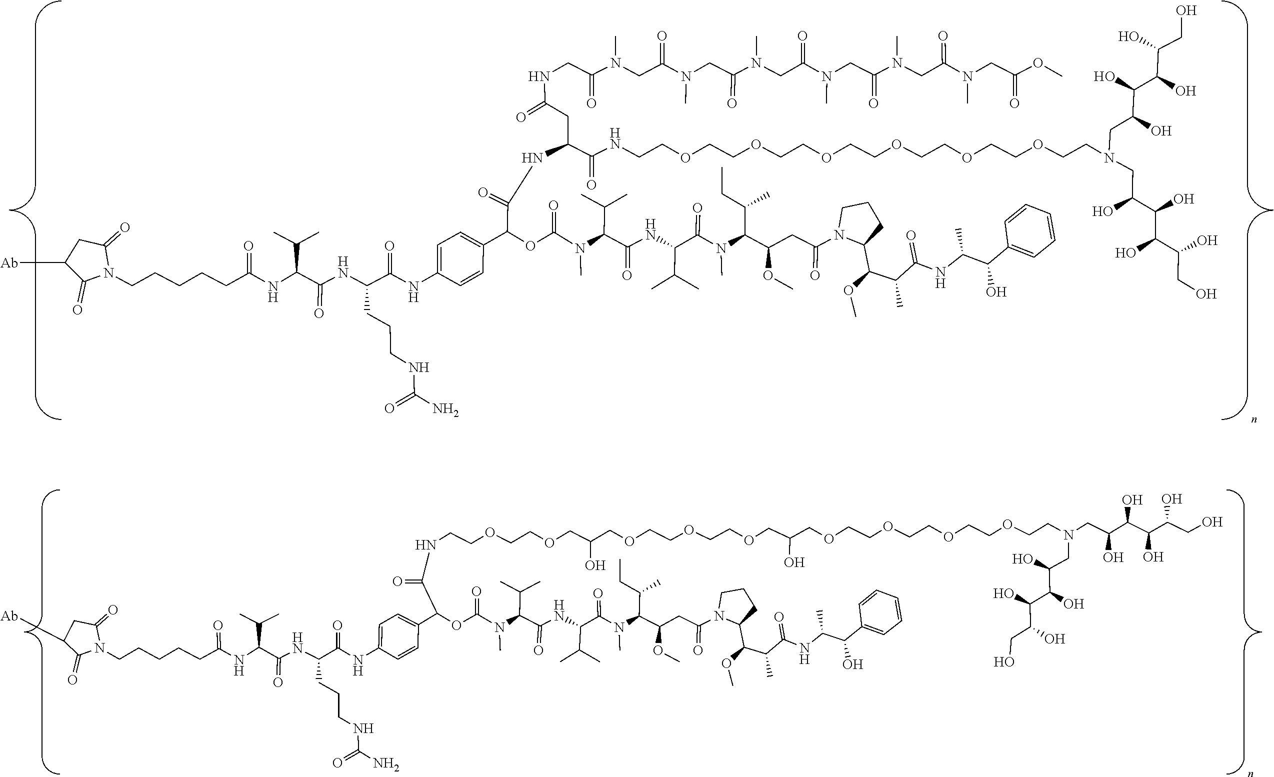

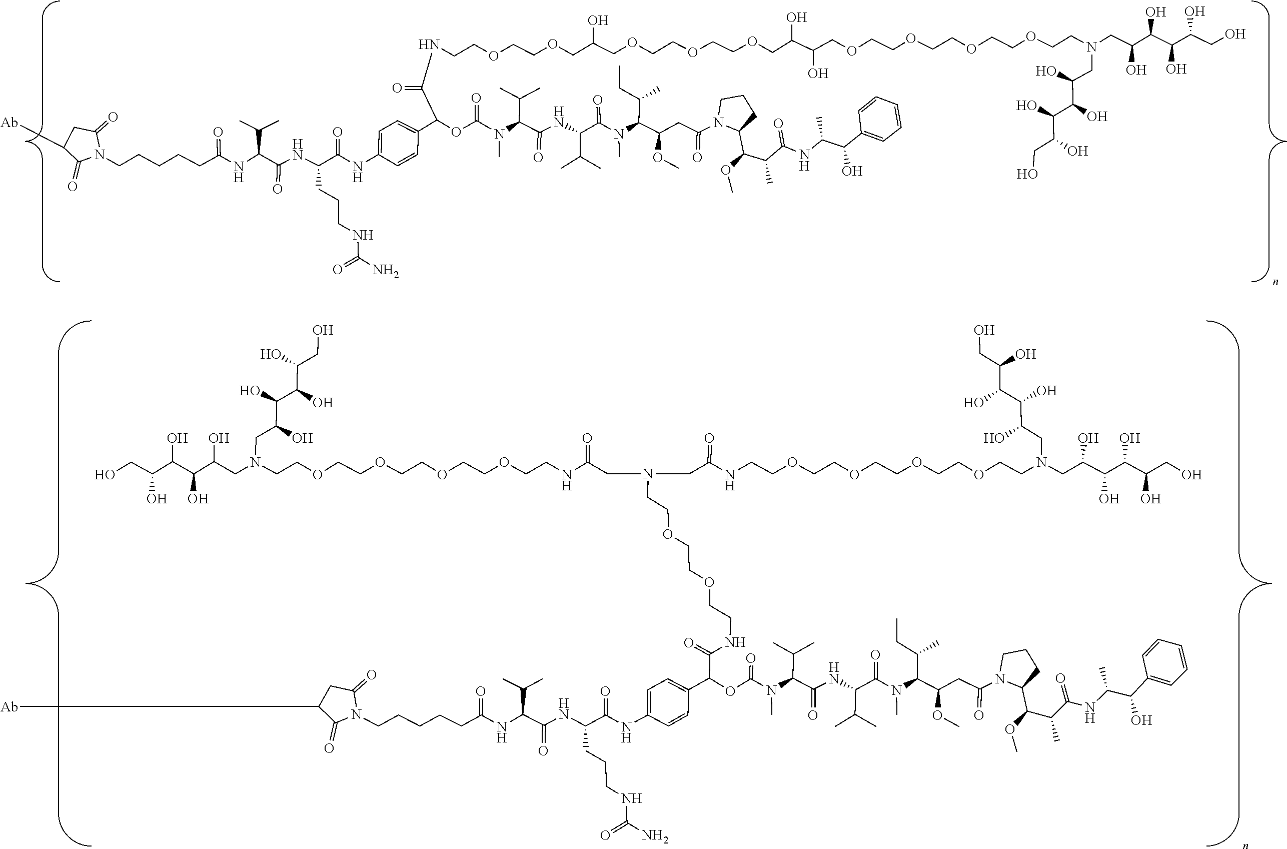

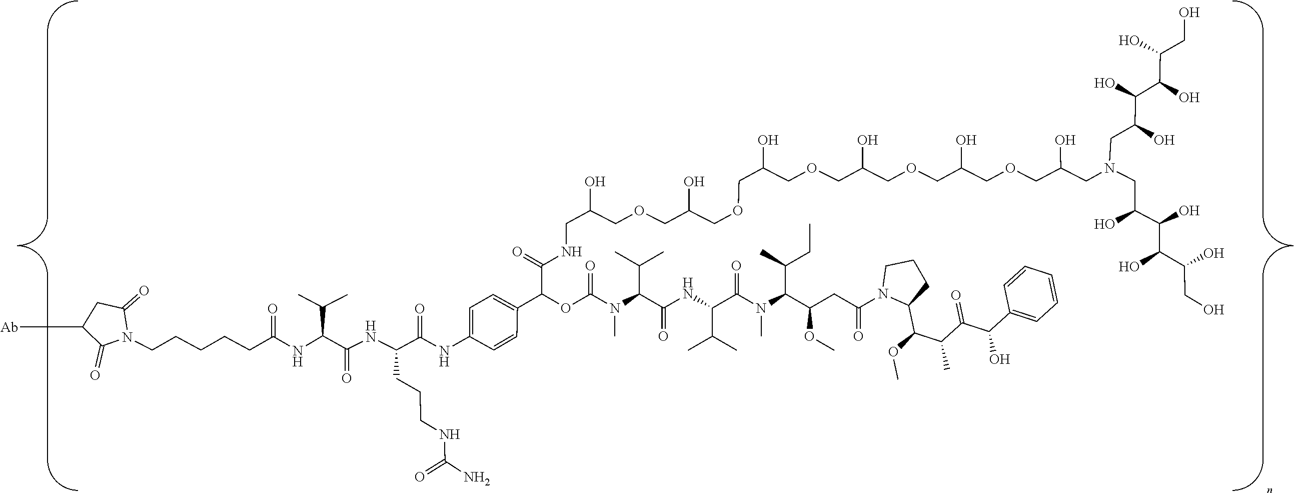

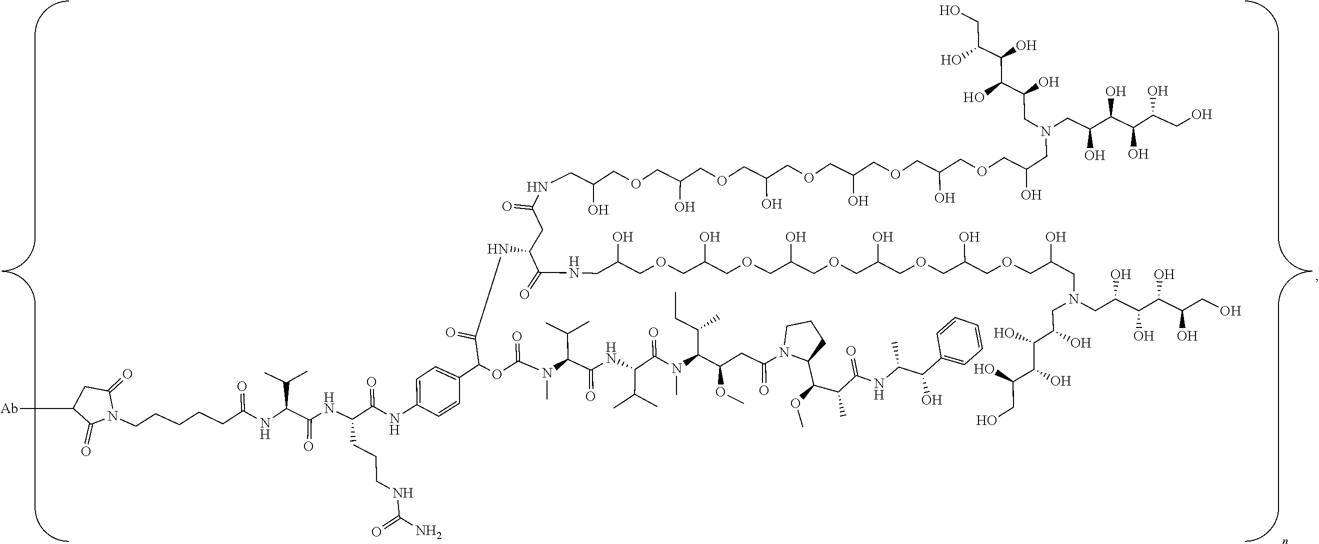

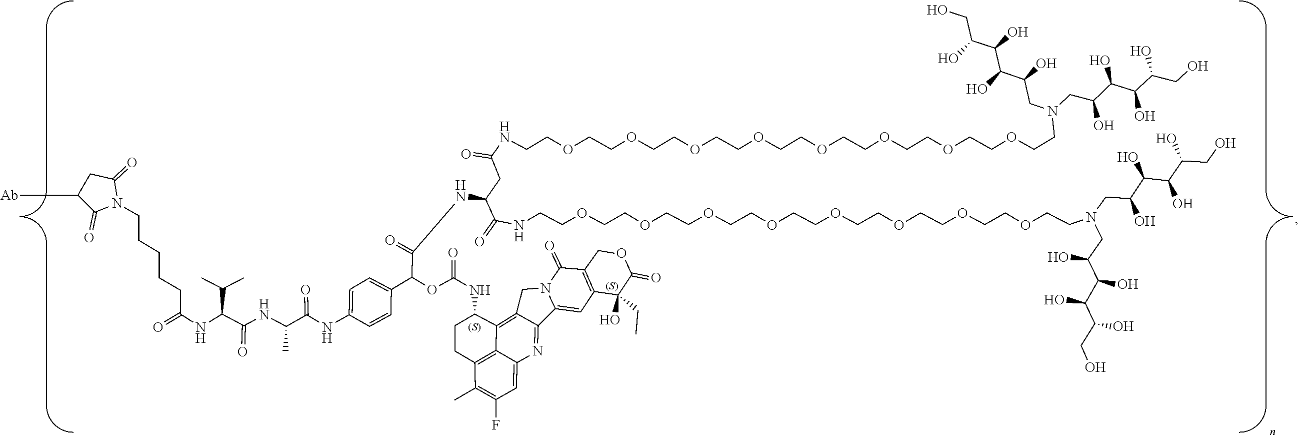

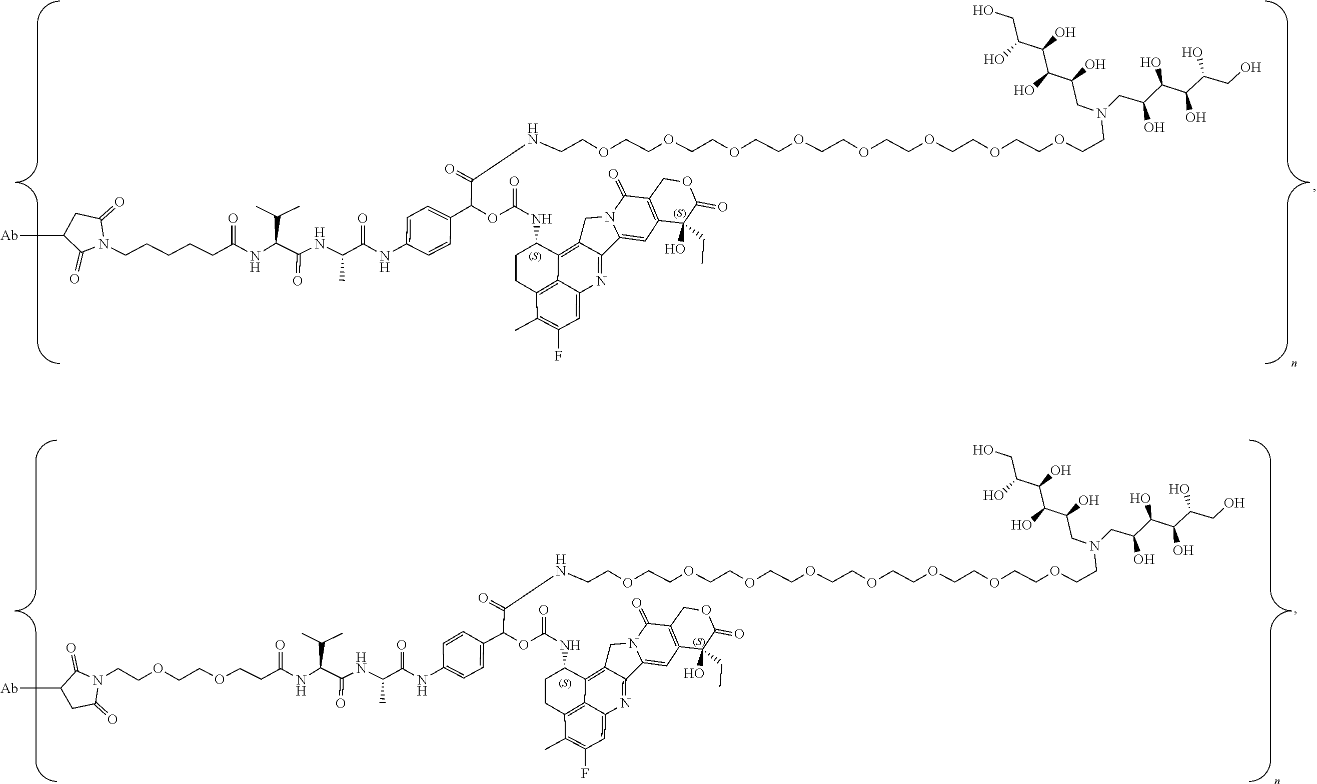

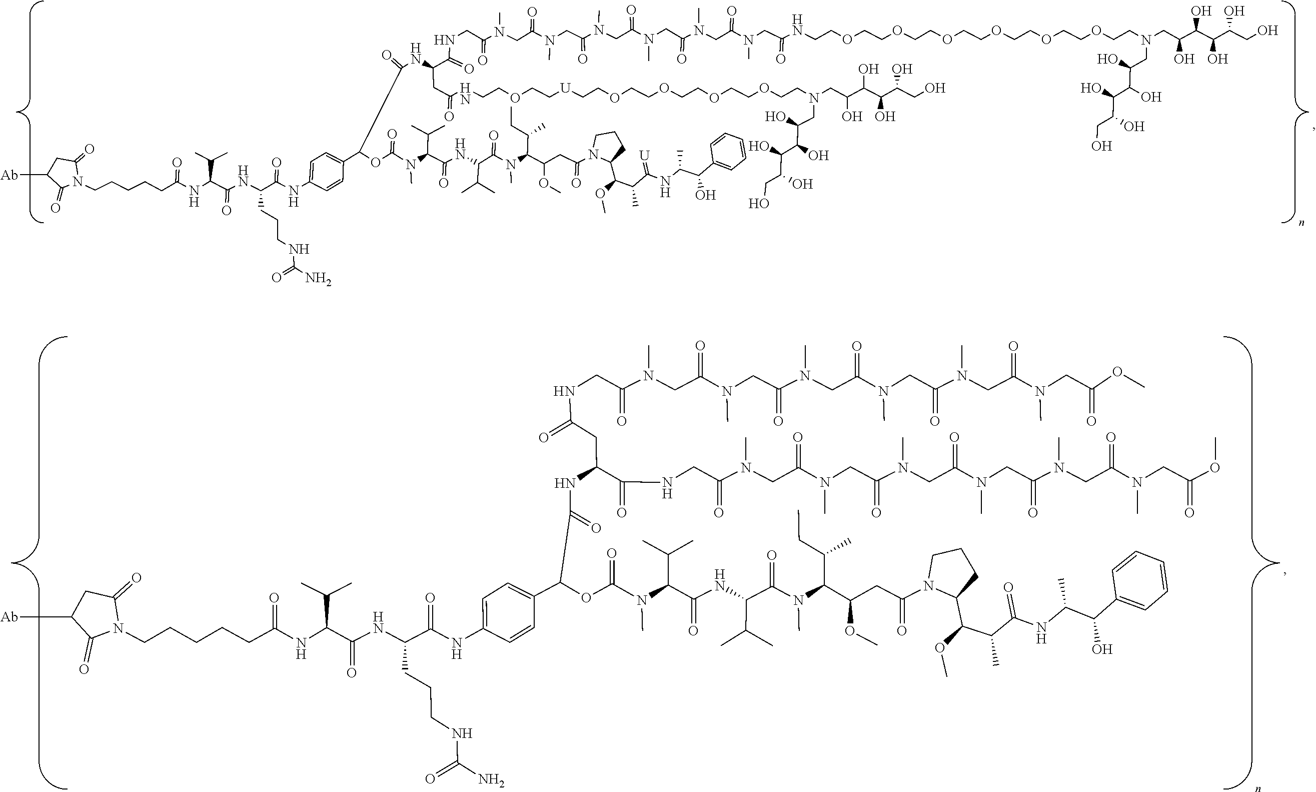

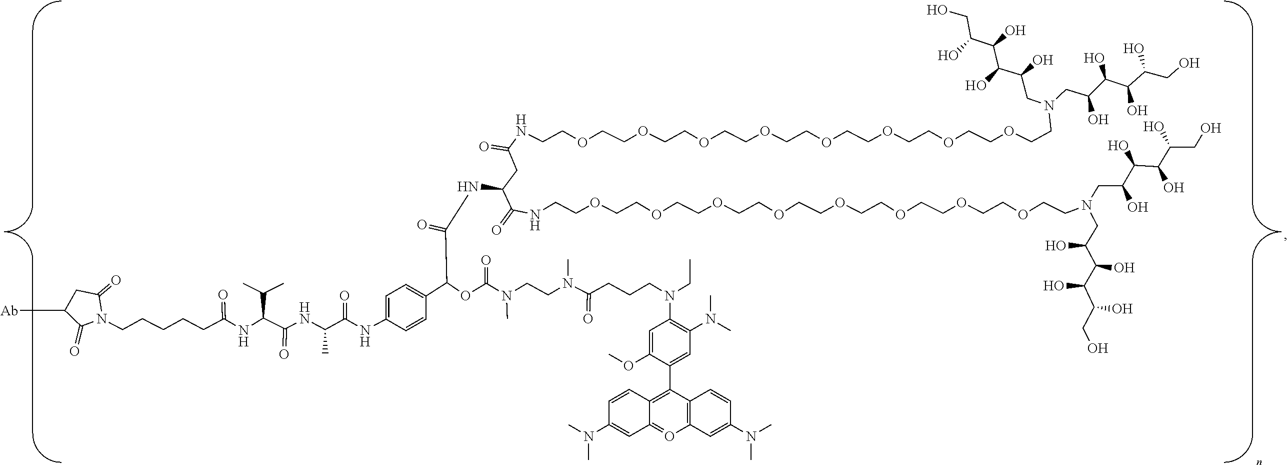

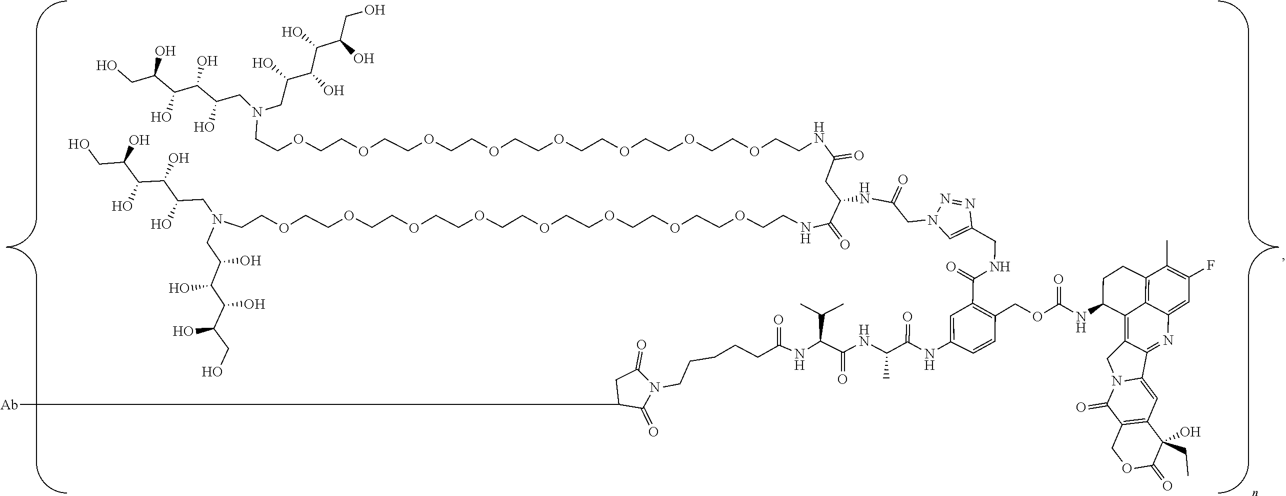

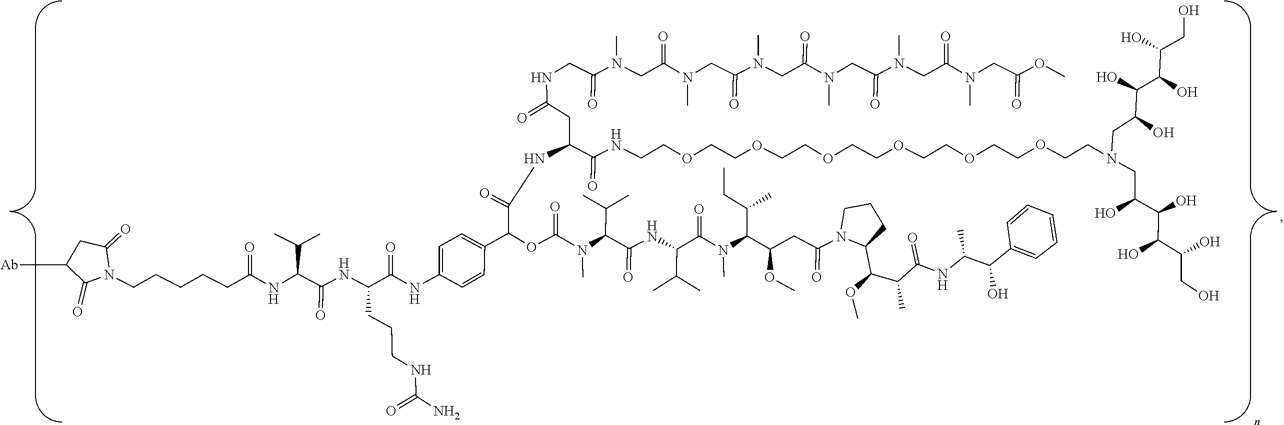

wherein Ab is a Targeting group and n is p^{load}.

In some embodiments, the conjugate is a stereoisomer of one of the above structures.

In some embodiments, provided are conjugates described above wherein the Targeting group specifically binds to a target molecule. In more specific embodiments, the target molecule is CD19, CD20, CD30, CD33, CD70, LIV-1 or EGFRv3. In more specific embodiments, the target molecule is CD19, CD20, CD30, CD33, CD70, LIV-1 EGFRv3, or HER2. For example, in some embodiments the Targeting group is selected from: a scFv1-ScFv2, a ScFv12-Fc-scFv22, an IgG-scFv, a DVD-Ig, a triomab/quadroma, a two-in-one IgG, a scFv2-Fc, a TandAb, and an scFv-HSA-scFv.

In other embodiments, provided are conjugates described above wherein the target molecule is a cancer associated antigen. For example, in some embodiments the target molecule is CD19, CD20, CD30, CD33, CD38, CA125, HER2, MUC-1, prostate-specific membrane antigen (PSMA), CD44 surface adhesion molecule, mesothelin (MLSN), carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-A1, IL-13R-a2, GD2, 1p19q, ABL1, AKT1, ALK, APC, AR, ATM, BRAF, BRCA1, BRCA2, cKIT, cMET, CSF1R, CTNNB1, FGFR1, FGFR2, FLT3, GNA11, GNAQ, GNAS, HRAS, IDH1, IDH2, JAK2, KDR (VEGFR2), KRAS, MGMT, MGMT-Me, MLH1, MPL, NOTCHI, NRAS, PDGFRA, Pgp, PIK3CA, PR, PTEN, RET, RRM1, SMO, SPARC, TLE3, TOP2A, TOPO1, TP53, TS, TUBB3, VHL, CDH1, ERBB4, FBXW7, HNF1A, JAK3, NPM1, PTPN11, RB1, SMAD4, SMARCBI, STK1, MLH1, MSH2, MSH6, PMS2, ROS1, ERCC1, 5T4 (TPBG), B7-H3, CCR7, CD105, CD22, CD46, CD47, CD56, CD70, CD71, CD79b, CDH6, CLDN6, CLDN18.2, CLEC12A, DLL3, DR5, ERBB3 (HER3), EPCAM, FOLR1, IGF1R, IL2RA (CD25), IL3RA, ITGB6, LIV-1, LRRC15, mesothelin (MSLN), NaPi2b (SLC34A2), nectin-4, PTK7, ROR1, SEZ6, SLC44A4, SLITRK6, Tissue Factor (TF), TROP2 or B7-H4.

In other embodiments, provided are conjugates described above wherein the Targeting group is an antibody, or fragment thereof, comprising rituximab (Rituxan®), trastuzumab (Herceptin®), pertuzumab (Perjeta®)), bevacizumab (Avastin®), ranibizumab (Lucentis®), cetuximab (Erbitux®), alemtuzumab (Campath®), panitumumab (Vectibix®), ibritumomab tiuxetan (Zevalin®), tositumomab (Bexxar®), ipilimumab, zalutumumab, dalotuzumab, figitumumab, ramucirumab, galiximab, farletuzumab, ocrelizumab, ofatumumab (Arzerra®), tositumumab, ibritumomab, the CD20 antibodies 2F2 (HuMax-CD20), 7D8, IgM2C6, IgG1 2C6, 11B8, B1, 2H7, LT20, 1FS or AT80, daclizumab (Zenapax®), or anti-LHRH receptor antibodies including clone A9E4, F1G4, AT2G7, GNRH03, or GNRHR2.

### Drug Loading

Conjugates can contain one or more Drug unit per Targeting group. The number of Drug units per Targeting group is referred to as drug loading. The drug loading of a Conjugate is represented by p^{load}, the average number of Drug units (drug molecules (e.g., cytotoxic agents)) per Targeting groups (e.g., an antibody or antigen binding portion or non-antibody scaffold or non-antibody protein) in a conjugate. For example, if p^{load} is about 4, the average drug loading taking into account all of the Targeting groups (e.g., antibodies or antigen binding portion or non-antibody scaffold or non-antibody proteins) present in the composition is about 4. In some embodiments, p^{load} ranges from about 3 to about 5, from about 3.6 to about 4.4, or from about 3.8 to about 4.2. In some embodiments, p^{load} can be about 3, about 4, or about 5. In some embodiments, p^{load} ranges from about 6 to about 8, more preferably from about 7.5 to about 8.4. In some embodiments, p^{load} can be about 6, about 7, or about 8. In some embodiments, p^{load} ranges from about 8 to about 16.

The average number of Drug units per Targeting group (e.g., antibody or antigen binding portion or non-antibody scaffold) in a preparation may be characterized by conventional means such as UV, mass spectroscopy, Capillary Electrophoresis (CE), and HPLC. The quantitative distribution of conjugates in terms of p^{load} may also be determined. In some instances, separation, purification, and characterization of homogeneous conjugates where p^{load} is a certain value from conjugates with other drug loadings may be achieved by means such as reverse phase HPLC or Hydrophobic Interaction Chromatography (HIC) HPLC.

### Attachment of Drug-Linkers to Antibodies, Antigen Binding Portions and Other Binding Agents (Including Non-Antibody Scaffolds)

Techniques for attaching Drug unit(s) to Targeting groups (such as antibodies or antigen binding portions thereof or non-antibody scaffolds) via linkers are well-known in the art. See, e.g., Alley et al., Current Opinion in Chemical Biology 2010 14:1-9; Senter, Cancer J., 2008, 14(3):154-169. In some embodiments, a Linker is first attached to a Drug unit (e.g., a cytotoxic agent(s), immune modulatory agent or other agent) and then the Drug-Linker(s) is attached to the Targeting group (e.g., an antibody or antigen binding portion thereof or non-antibody protein scaffold). In some embodiments, a Linker(s) is first attached to a Targeting group (e.g., an antibody or antigen binding portion thereof or non-antibody protein scaffold), and then a Drug unit is attached to a Linker. In the following discussion, the term Drug-Linker is used to exemplify attachment of Linkers or Drug-Linkers to Targeting groups; the skilled artisan will appreciate that the selected attachment method can be determined according to Linker and the Drug unit. In some embodiments, a Drug unit is attached to a Targeting group via a Linker in a manner that reduces the activity of the Drug unit until it is released from the conjugate (e.g., by hydrolysis, by proteolytic degradation or by a cleaving agent.).

Generally, a conjugate may be prepared by several routes employing organic chemistry reactions, conditions, and reagents known to those skilled in the art, including: (1) reaction of a nucleophilic group of a Targeting group (e.g., an antibody or antigen binding portion thereof or non-antibody protein scaffold) with a bivalent Linker to form a Targeting group-Linker intermediate via a covalent bond, followed by reaction with a Drug unit; and (2) reaction of a nucleophilic group of a Drug unit with a bivalent Linker, to form Drug-Linker, via a covalent bond, followed by reaction with a nucleophilic group of a Targeting group. Exemplary methods for preparing conjugates via the latter route are described in U.S. Pat. No. 7,498,298, which is expressly incorporated herein by reference.

Nucleophilic groups on Targeting groups such as antibodies, antigen binding portions and other binding agents (including non-antibody scaffolds) include, but are not limited to: (i)N-terminal amine groups, (ii) side chain amine groups, e.g. lysine, (iii) side chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on Linkers including: (i) active esters such as NHS esters, HOBt esters, haloformates, and acid halides; (ii) alkyl and benzyl halides such as haloacetamides; and (iii) aldehydes, ketones, carboxyl, and maleimide groups. Certain Targeting groups, such as antibodies (and antigen binding portions and other binding agents (including non-antibody scaffolds)) have reducible interchain disulfides, i.e., cysteine bridges. Antibodies (and antigen binding portions and other binding agents (including non-antibody scaffolds)) may be made reactive for conjugation with Linkers by treatment with a reducing agent such as DTT (dithiothreitol) or tricarbonylethylphosphine (TCEP), such that the antibody is fully or partially reduced. Each cysteine bridge will thus form, theoretically, two reactive thiol nucleophiles. Additional nucleophilic groups can be introduced into Targeting groups such as antibodies (and antigen binding portions and other binding agents (including non-antibody scaffolds)) through modification of lysine residues, e.g., by reacting lysine residues with 2-iminothiolane (Traut's reagent), resulting in conversion of an amine into a thiol. Reactive thiol groups may also be introduced into a Targeting group (such as an antibody and antigen binding portions and other binding agents (including non-antibody scaffolds)) by introducing one, two, three, four, or more cysteine residues (e.g., by preparing antibodies, antigen binding portions and other binding agents (including non-antibody scaffolds) comprising one or more non-native cysteine amino acid residues).

Conjugates may also be produced by reaction between an electrophilic group on a Targeting group, such as an aldehyde or ketone carbonyl group, with a nucleophilic group on a Linker reagent. Useful nucleophilic groups on a linker reagent include, but are not limited to, hydrazide, oxime, amino, hydrazine, thiosemicarbazone, hydrazine carboxyl, and arylhydrazide. In an embodiment, an antibody (or antigen binding portion thereof or other binding agent (including non-antibody scaffolds)) is modified to introduce electrophilic moieties that are capable of reacting with nucleophilic substituents on a Linker. In another embodiment, the sugars of glycosylated antibodies may be oxidized, e.g. with periodate oxidizing reagents, to form aldehyde or ketone groups which may react with the amine group of a Linker. The resulting imine Schiff base groups may form a stable linkage, or may be reduced, e.g., by borohydride reagents to form stable amine linkages. In one embodiment, reaction of the carbohydrate portion of a glycosylated antibody with either galactose oxidase or sodium meta-periodate may yield carbonyl (aldehyde and ketone) groups in the antibody (or antigen binding portion thereof or other binding agent (including non-antibody scaffolds)) that can react with appropriate groups on the Linker (see, e.g., Hermanson, Bioconjugate Techniques). In another embodiment, Targeting groups such as antibodies containing N-terminal serine or threonine residues can react with sodium meta-periodate, resulting in production of an aldehyde in place of the first amino acid (Geoghegan & Stroh, (1992) Bioconjugate Chem. 3:138-146; U.S. Pat. No. 5,362,852). Such an aldehyde can be reacted with a Linker.

Exemplary nucleophilic groups on a Drug unit, such as a cytotoxic agent, include, but are not limited to: amine, thiol, hydroxyl, hydrazide, oxime, hydrazine, thiosemicarbazone, hydrazine carboxyl, and arylhydrazide groups capable of reacting to form covalent bonds with electrophilic groups on a Linker(s) including: (i) active esters such as NHS esters, HOBt esters, haloformates, and acid halides; (ii) alkyl and benzyl halides such as haloacetamides; (iii) aldehydes, ketones, carboxyl, and maleimide groups.

In some embodiments, a Drug-Linker is attached to an interchain cysteine residue(s) of an antibody (or antigen binding portion thereof or other binding agent (including non-antibody scaffolds)). See, e.g., WO2004/010957 and WO2005/081711. In such embodiments, the Linker typically comprises a maleimide group for attachment to the cysteine residues of an interchain disulfide. In some embodiments, a Linker or Drug-Linker is attached to a cysteine residue(s) of an antibody or antigen binding portion thereof as described in U.S. Pat. Nos. 7,585,491 or 8,080,250. The drug loading of the resulting conjugate typically ranges from 1 to 8 or 1 to 16.

In some embodiments, a Linker or Drug-Linker is attached to a lysine or cysteine residue(s) of an antibody (or antigen binding portion thereof or other binding agent) as described in WO2005/037992 or WO2010/141566. The drug loading of the resulting conjugate typically ranges from 1 to 8.

In some embodiments, engineered cysteine residues, poly-histidine sequences, glycoengineering tags, or transglutaminase recognition sequences can be used for site-specific attachment of linkers or drug-linkers to antibodies or antigen binding portions thereof or other binding agents (including non-antibody scaffolds).

In some embodiments, a Drug-Linker(s) is attached to an engineered cysteine residue at an Fc residue other than an interchain disulfide. In some embodiments, a Drug-Linker(s) is attached to an engineered cysteine introduced into an IgG (typically an IgG1) at position 118, 221, 224, 227, 228, 230, 231, 223, 233, 234, 235, 236, 237, 238, 239, 240, 241, 243, 244, 245, 247, 249, 250, 258, 262, 263, 264, 265, 266, 267, 268, 269, 270, 271, 272, 273, 275, 276, 278, 280, 281, 283, 285, 286, 291, 292, 293, 294, 295, 296, 297, 298, 299, 300, 302, 305, 313, 318, 323, 324, 325, 327, 328, 329, 330, 331, 332, 333, 335, 336, 396, and/or 428, of the heavy chain and/or to a light chain at position 106, 108, 142 (light chain), 149 (light chain), and/or position V205, according to the EU numbering of Kabat. An exemplary substitution for site specific conjugation using an engineered cysteine is S239C (see, e.g., US 20100158909; numbering of the Fc region is according to the EU index).

In some embodiments, a Linker or Drug-Linker(s) is attached to one or more introduced cysteine residues of an antibody (or antigen binding portion thereof or other binding agent (including non-antibody scaffolds)) as described in WO2006/034488, WO2011/156328 and/or WO2016040856.

In some embodiments, an exemplary substitution for site specific conjugation using bacterial transglutaminase is N297S or N297Q of the Fc region. In some embodiments, a Linker or Drug-Linker(s) is attached to the glycan or modified glycan of an antibody or antigen binding portion or a glycoengineered antibody (or other binding agent (including non-antibody scaffolds)). See, e.g., WO2017/147542, WO2020/123425, WO2020/245229, WO2014/072482; WO2014//065661, WO2015/057066 and WO2016/022027; the disclosure of which are incorporated by reference herein.

In some embodiments, a Linker or Drug-Linker is attached to an antibody, antigen binding portion or other binding agent (including non-antibody scaffolds) via Sortase A linker. A Sortase A linker can be created by a Sortase A enzyme fusing an LPXTG recognition motif (SEQ ID NO: 5) to an N-terminal GGG motif to regenerate a native amide bond.

In some embodiments, a Linker or Drug-Linker is attached to an antibody, antigen binding portion or other binding agent (including non-antibody scaffolds) using SMARTag Technology, in which a bioorthogonal aldehyde handle is introduced through the oxidation of a cysteine residue, embedded in a specific peptide sequence (CxPxR), to an aldehyde-bearing formylglycine (fGly). This enzymatic modification is carried out by the formylglycine-generating enzyme (FGE). See, e.g., Liu et al., Methods Mol. Biol. 2033:131-147 (2019).

In some embodiments, a Linker or Drug-Linker is attached to an antibody, antigen binding portion or other binding agent (including non-antibody scaffolds) using cysteine conjugation with quaternized vinyl- and alkynyl-pyridine reagents. See, e.g., Matos et al., Angew Chem. Int. Ed. Engl. 58:6640-6644 (2019).

In other embodiments, a Linker or Drug-Linker is attached to an antibody, antigen binding portion or other binding agent (including non-antibody scaffolds) using bis-maleimide, C-lock, or K-lock methodologies.

### Pharmaceutical Compositions

Other aspects of the conjugates relate to compositions comprising active ingredients, including any of the conjugates described herein. In some embodiments, the composition is a pharmaceutical composition. As used herein, the term “pharmaceutical composition” refers to an active agent in combination with a pharmaceutically acceptable carrier accepted for use in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The preparation of a pharmacological composition that contains active ingredients dissolved or dispersed therein is well understood in the art and need not be limited based on any particular formulation. Typically, such compositions are prepared as injectable either as liquid solutions or suspensions; however, solid forms suitable for rehydration, or suspensions, in liquid prior to use can also be prepared. A preparation can also be emulsified or presented as a liposome composition. A conjugate can be mixed with excipients that are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, a pharmaceutical composition can contain minor amounts of auxiliary substances such as wetting or emulsifying agents, pH buffering agents and the like which enhance or maintain the effectiveness of the active ingredient (e.g., a conjugate).

The pharmaceutical compositions as described herein can include pharmaceutically acceptable salts of the components therein. Pharmaceutically acceptable salts include the acid addition salts (formed with the free amino groups of a polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary liquid carriers are sterile aqueous solutions that contain the active ingredients (e.g., a conjugate) and water, and may contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent that will be effective in the treatment of a particular disorder or condition will depend on the nature of the disorder or condition, and can be determined by standard clinical techniques.

In some embodiments, a pharmaceutical composition comprising a conjugate can be a lyophilisate.

In some embodiments, a syringe comprising a therapeutically effective amount of a conjugate is provided.

### Treatment Methods

In some embodiments, provided are methods of treating a subject, comprising administering to the subject a conjugate described herein or a pharmaceutical composition described herein. For example, in some embodiments the subject has cancer or an autoimmune disease and the conjugate binds to the target antigen associated with the cancer or autoimmune disease.

In some embodiments, provided are methods of treating cancer comprising administering a conjugate. In some embodiments, the subject is in need of treatment for a cancer and/or a malignancy. In some embodiments, the method is for treating a subject having a cancer or malignancy.

The methods described herein include administering a therapeutically effective amount of a conjugate to a subject having a cancer or malignancy. As used herein, the phrases “therapeutically effective amount,” “effective amount,” or “effective dose” refer to an amount of a conjugate that provides a therapeutic benefit in the treatment of, management of or prevention of relapse of a cancer or malignancy, e.g., an amount that provides a statistically significant decrease in at least one symptom, sign, or marker of a tumor or malignancy. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

The terms “cancer” and “malignancy” refer to an uncontrolled growth of cells which interferes with the normal functioning of the bodily organs and systems. A cancer or malignancy may be primary or metastatic, i.e. that is it has become invasive, seeding tumor growth in tissues remote from the original tumor site. A “tumor” refers to an uncontrolled growth of cells which interferes with the normal functioning of the bodily organs and systems. A subject that has a cancer is a subject having objectively measurable cancer cells present in the subject's body. Included in this definition are benign tumors and malignant cancers, as well as potentially dormant tumors and micro-metastases. Cancers that migrate from their original location and seed other vital organs can eventually lead to the death of the subject through the functional deterioration of the affected organs. Hematologic malignancies (hematopoietic cancers), such as leukemias and lymphomas, are able to, for example, out-compete the normal hematopoietic compartments in a subject, thereby leading to hematopoietic failure (in the form of anemia, thrombocytopenia and neutropenia) ultimately causing death.

Examples of cancers include, but are not limited to, carcinomas, lymphomas, blastomas, sarcomas, and leukemias. More particular examples of such cancers include, but are not limited to, basal cell cancer, biliary tract cancer, bladder cancer, bone cancer, brain and CNS cancer, breast cancer (e.g., triple negative breast cancer), cancer of the peritoneum, cervical cancer; cholangiocarcinoma, choriocarcinoma, chondrosarcoma, colon and rectum cancer (colorectal cancer), connective tissue cancer, cancer of the digestive system, endometrial cancer, esophageal cancer, eye cancer, cancer of the head and neck, gastric cancer (including gastrointestinal cancer and stomach cancer), glioblastoma (GBM), hepatic cancer, hepatoma, intra-epithelial neoplasm, kidney or renal cancer (e.g., clear cell cancer), larynx cancer, leukemia, liver cancer, lung cancer (e.g., small-cell lung cancer, non-small cell lung cancer, adenocarcinoma of the lung, and squamous cancer of the lung), lymphoma including Hodgkin's and non-Hodgkin's lymphoma, melanoma, mesothelioma, myeloma, neuroblastoma, oral cavity cancer (e.g., lip, tongue, mouth, and pharynx), ovarian cancer, pancreatic cancer, prostate cancer, retinoblastoma, rhabdomyosarcoma, cancer of the respiratory system, salivary gland cancer, sarcoma, skin cancer, squamous cell cancer, testicular cancer, thyroid cancer, uterine or endometrial cancer, uterine serious cancer, cancer of the urinary system, vulval cancer; as well as other carcinomas and sarcomas, as well as B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, and Waldenstrom's Macroglobulinemia), chronic lymphocytic leukemia (CLL), acute lymphoblastic leukemia (ALL), Hairy cell leukemia, chronic myeloblastic leukemia, and post-transplant lymphoproliferative disorder (PTLD), as well as abnormal vascular proliferation associated with phakomatoses, edema (such as that associated with brain tumors), and Meigs' syndrome.

It is contemplated that the methods herein reduce tumor size or tumor burden in the subject, and/or reduce metastasis in the subject. In various embodiments, tumor size in the subject is decreased by about 25-50%, about 40-70% or about 50-90% or more. In various embodiments, the methods reduce the tumor size by 10%, 20%, 30% or more. In various embodiments, the methods reduce tumor size by 10%, 15%, 20%, 25%, 30%, 35%, 40%, 45%, 50%, 55%, 60%, 65%, 70%, 75%, 80%, 85%, 90%, 95% or 100%.

As used herein, a “subject” refers to a human or animal. Usually, the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments, the subject is a mammal, e.g., a primate, e.g., a human. The terms, “patient,” “individual,” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used, for example, as subjects that represent animal models of, for example, various cancers. In addition, the methods described herein can be used to treat domesticated animals and/or pets. A subject can be male or female. In certain embodiments, the subject is a human.

In some embodiments, a subject can be one who has been previously diagnosed with or identified as suffering from a cancer and in need of treatment, but need not have already undergone treatment for the cancer. In some embodiments, a subject can also be one who has not been previously diagnosed as having a cancer in need of treatment. In some embodiments, a subject can be one who exhibits one or more risk factors for a condition or one or more complications related to a cancer or a subject who does not exhibit risk factors. A “subject in need” of treatment for a cancer particular can be a subject having that condition or diagnosed as having that condition. In other embodiments, a subject “at risk of developing” a condition refers to a subject diagnosed as being at risk for developing the condition or at risk for having the condition again.

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, reduction in cancer cells in the subject, alleviation of one or more symptom(s), diminishment of extent of the deficit, stabilized (i.e., not worsening) state of a cancer or malignancy, delay or slowing of tumor growth and/or metastasis, and an increased lifespan as compared to that expected in the absence of treatment. As used herein, the term “administering,” refers to providing a conjugate as described herein to a subject by a method or route which results in binding of the conjugate to cancer cells or malignant cells. Similarly, a pharmaceutical composition comprising a conjugate as described herein can be administered by any appropriate route which results in an effective treatment in the subject.

The dosage ranges for a conjugate depend upon the potency, and encompass amounts large enough to produce the desired effect e.g., slowing of tumor growth or a reduction in tumor size. The dosage should not be so large as to cause unacceptable adverse side effects. Generally, the dosage will vary with the age, condition, and sex of the subject and can be determined by one of skill in the art. The dosage can also be adjusted by the individual physician in the event of any complication. In some embodiments, the dosage ranges from 0.1 mg/kg body weight to 10 mg/kg body weight. In some embodiments, the dosage ranges from 0.5 mg/kg body weight to 15 mg/kg body weight. In some embodiments, the dose range is from 0.5 mg/kg body weight to 5 mg/kg body weight. Alternatively, the dose range can be titrated to maintain serum levels between 1 μg/mL and 1000 μg/mL. For systemic administration, subjects can be administered a therapeutic amount, such as, e.g. 0.1 mg/kg, 0.5 mg/kg, 1.0 mg/kg, 2.0 mg/kg, 2.5 mg/kg, 5 mg/kg, 10 mg/kg, 12 mg/kg or more.

Administration of the doses recited above can be repeated. In a preferred embodiment, the doses recited above are administered weekly, biweekly, every three weeks or monthly for several weeks or months. The duration of treatment depends upon the subject's clinical progress and responsiveness to treatment.

In some embodiments, a dose can be from about 0.1 mg/kg to about 100 mg/kg. In some embodiments, a dose can be from about 0.1 mg/kg to about 25 mg/kg. In some embodiments, a dose can be from about 0.1 mg/kg to about 20 mg/kg. In some embodiments, a dose can be from about 0.1 mg/kg to about 15 mg/kg. In some embodiments, a dose can be from about 0.1 mg/kg to about 12 mg/kg. In some embodiments, a dose can be from about 1 mg/kg to about 100 mg/kg. In some embodiments, a dose can be from about 1 mg/kg to about 25 mg/kg. In some embodiments, a dose can be from about 1 mg/kg to about 20 mg/kg. In some embodiments, a dose can be from about 1 mg/kg to about 15 mg/kg. In some embodiments, a dose can be from about 1 mg/kg to about 12 mg/kg. In some embodiments, a dose can be from about 1 mg/kg to about 10 mg/kg.

In some embodiments, a dose can be administered intravenously. In some embodiments, an intravenous administration can be an infusion occurring over a period of from about 10 minutes to about 4 hours. In some embodiments, an intravenous administration can be an infusion occurring over a period of from about 30 minutes to about 90 minutes.

In some embodiments, a dose can be administered weekly. In some embodiments, a dose can be administered bi-weekly. In some embodiments, a dose can be administered about every 2 weeks. In some embodiments, a dose can be administered about every 3 weeks. In some embodiments, a dose can be administered every four weeks.

In some embodiments, a total of from about 2 to about 10 doses are administered to a subject. In some embodiments, a total of 4 doses are administered. In some embodiments, a total of 5 doses are administered. In some embodiments, a total of 6 doses are administered. In some embodiments, a total of 7 doses are administered. In some embodiments, a total of 8 doses are administered. In some embodiments, a total of 9 doses are administered. In some embodiments, a total of 10 doses are administered. In some embodiments, a total of more than 10 doses are administered.

Pharmaceutical compositions containing a conjugate can be administered in a unit dose. The term “unit dose” when used in reference to a pharmaceutical composition refers to physically discrete units suitable as unitary dosage for the subject, each unit containing a predetermined quantity of active material (e.g., conjugate), calculated to produce the desired therapeutic effect in association with the required physiologically acceptable diluent, i.e., carrier, or vehicle.

In some embodiments, the conjugates as described herein can be used in a method(s) comprising administering a conjugate to a subject in need thereof, such as a subject having an autoimmune disease.

In some embodiments, provided are methods of treating an autoimmune disease comprising administering a conjugate as described herein. In some embodiments, the subject is in need of treatment for an autoimmune disease. The methods described herein include administering a therapeutically effective amount of a conjugate to a subject having an autoimmune disease. As used herein, the phrase “therapeutically effective amount,” “effective amount,” or “effective dose” refers to an amount of a conjugate as described herein that provides a therapeutic benefit in the treatment of, management of or prevention of relapse of an autoimmune disease, e.g., an amount that provides a statistically significant decrease in at least one symptom, sign, or marker of an autoimmune disease. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

The term “autoimmune disease” refers to an immunological disorder characterized by inappropriate activation of immune cells (e.g., lymphocytes or dendritic cells), that interferes with the normal functioning of the bodily organs and systems. Examples of autoimmune disease include, but are not limited to, rheumatoid arthritis, psoriatic arthritis, autoimmune demyelinative diseases (e.g., multiple sclerosis, allergic encephalomyelitis), endocrine ophthalmopathy, uveoretinitis, systemic lupus erythematosus, myasthenia gravis, Grave's disease,

glomerulonephritis, autoimmune hepatological disorder, inflammatory bowel disease (e.g., Crohn's disease), anaphylaxis, allergic reaction, Sjogren's syndrome, type I diabetes mellitus, primary biliary cirrhosis, Wegener's granulomatosis, fibromyalgia, polymyositis, dermatomyositis, multiple endocrine failure, Schmidt's syndrome, autoimmune uveitis, Addison's disease, adrenalitis, thyroiditis, Hashimoto's thyroiditis, autoimmune thyroid disease, pernicious anemia, gastric atrophy, chronic hepatitis, lupoid hepatitis, atherosclerosis, subacute cutaneous lupus erythematosus, hypoparathyroidism, Dressler's syndrome, autoimmune thrombocytopenia, idiopathic thrombocytopenic purpura, hemolytic anemia, pemphigus vulgaris, pemphigus, dermatitis herpetiformis, alopecia areata, pemphigoid, scleroderma, progressive systemic sclerosis, CREST syndrome (calcinosis, Raynaud's phenomenon, esophageal dysmotility, sclerodactyl), and telangiectasia), male and female autoimmune infertility, ankylosing spondolytis, ulcerative colitis, mixed connective tissue disease, polyarteritis nodosa, systemic necrotizing vasculitis, atopic dermatitis, atopic rhinitis, Goodpasture's syndrome, Chagas' disease, sarcoidosis, rheumatic fever, asthma, recurrent abortion, anti-phospholipid syndrome, farmer's lung, erythema multiforme, post cardiotomy syndrome, Cushing's syndrome, autoimmune chronic active hepatitis, bird-fancier's lung, toxic epidermal necrolysis, Alport's syndrome, alveolitis, allergic alveolitis, fibrosing alveolitis, interstitial lung disease, erythema nodosum, pyoderma gangrenosum, transfusion reaction, Takayasu's arteritis, polymyalgia rheumatica, temporal arteritis, schistosomiasis, giant cell arteritis, ascariasis, aspergillosis, Samter's syndrome, eczema, lymphomatoid granulomatosis, Behcet's disease, Caplan's syndrome, Kawasaki's disease, dengue, encephalomyelitis, endocarditis, endomyocardial fibrosis, endophthalmitis, erythema elevatum et diutinum, psoriasis, erythroblastosis fetalis, eosinophilic faciitis, Shulman's syndrome, Felty's syndrome, filariasis, cyclitis, chronic cyclitis, heterochronic cyclitis, Fuch's cyclitis, IgA nephropathy, Henoch-Schonlein purpura, graft versus host disease, transplantation rejection, cardiomyopathy, Eaton-Lambert syndrome, relapsing polychondritis, cryoglobulinemia, Waldenstrom's macroglobulemia, Evan's syndrome, and autoimmune gonadal failure.

In some embodiments, the methods described herein encompass treatment of disorders of B lymphocytes (e.g., systemic lupus erythematosus, Goodpasture's syndrome, rheumatoid arthritis, and type I diabetes), Th1-lymphocytes (e.g., rheumatoid arthritis, multiple sclerosis, psoriasis, Sjorgren's syndrome, Hashimoto's thyroiditis, Grave's disease, primary biliary cirrhosis, Wegener's granulomatosis, tuberculosis, or graft versus host disease), or Th2-lymphocytes (e.g., atopic dermatitis, systemic lupus erythematosus, atopic asthma, rhinoconjunctivitis, allergic rhinitis, Omenn's syndrome, systemic sclerosis, or chronic graft versus host disease). Generally, disorders involving dendritic cells involve disorders of Th1-lymphocytes or Th2-lymphocytes.

As used herein, a “subject” refers to a human or animal. Usually, the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments, the subject is a mammal, e.g., a primate, e.g., a human. The terms, “patient,” “individual,” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used, for example, as subjects that represent animal models of, for example, various autoimmune diseases. In addition, the methods described herein can be used to treat domesticated animals and/or pets. A subject can be male or female. In certain embodiments, the subject is a human.

In some embodiments, a subject can be one who has been previously diagnosed with or identified as suffering from an autoimmune disease and in need of treatment, but need not have already undergone treatment for the autoimmune disease. In some embodiments, a subject can also be one who has not been previously diagnosed as having an autoimmune disease in need of treatment. In some embodiments, a subject can be one who exhibits one or more risk factors for a condition or one or more complications related to an autoimmune disease or a subject who does not exhibit risk factors. A “subject in need” of treatment for an autoimmune disease particular can be a subject having that condition or diagnosed as having that condition. In other embodiments, a subject “at risk of developing” a condition refers to a subject diagnosed as being at risk for developing the condition or at risk for having the condition again (e.g., an autoimmune disease).

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, reduction in autoimmune cells in the subject, alleviation of one or more symptom(s), diminishment of extent of the deficit, stabilized (i.e., not worsening) state of an autoimmune disease, delay or slowing of progression of an autoimmune disease, and an increased lifespan as compared to that expected in the absence of treatment. As used herein, the term “administering,” refers to providing a conjugate as described herein to a subject by a method or route which results in binding of the conjugate to target autoimmune cells. Similarly, a pharmaceutical composition comprising a conjugate as described herein can be administered by any appropriate route which results in an effective treatment in the subject.

The dosage ranges for a conjugate depend upon the potency, and encompass amounts large enough to produce the desired effect e.g., slowing of progression of an autoimmune disease or a reduction of symptoms. The dosage should not be so large as to cause unacceptable adverse side effects. Generally, the dosage will vary with the age, condition, and sex of the subject and can be determined by one of skill in the art. The dosage can also be adjusted by the individual physician in the event of any complication. In some embodiments, the dosage ranges from 0.1 mg/kg body weight to 10 mg/kg body weight. In some embodiments, the dosage ranges from 0.5 mg/kg body weight to 15 mg/kg body weight. In some embodiments, the dose range is from 0.5 mg/kg body weight to 5 mg/kg body weight. Alternatively, the dose range can be titrated to maintain serum levels between 1 μg/mL and 1000 μg/mL. For systemic administration, subjects can be administered a therapeutic amount, such as, e.g. 0.1 mg/kg, 0.5 mg/kg, 1.0 mg/kg, 2.0 mg/kg, 2.5 mg/kg, 5 mg/kg, 10 mg/kg, 12 mg/kg or more.

Administration of the doses recited above can be repeated. In a preferred embodiment, the doses recited above are administered weekly, biweekly, every three weeks or monthly for several weeks or months. The duration of treatment depends upon the subject's clinical progress and responsiveness to treatment.

In some embodiments, a dose can be from about 0.1 mg/kg to about 100 mg/kg. In some embodiments, a dose can be from about 0.1 mg/kg to about 25 mg/kg. In some embodiments, a dose can be from about 0.1 mg/kg to about 20 mg/kg. In some embodiments, a dose can be from about 0.1 mg/kg to about 15 mg/kg. In some embodiments, a dose can be from about 0.1 mg/kg to about 12 mg/kg. In some embodiments, a dose can be from about 1 mg/kg to about 100 mg/kg. In some embodiments, a dose can be from about 1 mg/kg to about 25 mg/kg. In some embodiments, a dose can be from about 1 mg/kg to about 20 mg/kg. In some embodiments, a dose can be from about 1 mg/kg to about 15 mg/kg. In some embodiments, a dose can be from about 1 mg/kg to about 12 mg/kg. In some embodiments, a dose can be from about 1 mg/kg to about 10 mg/kg.

In some embodiments, a dose can be administered intravenously. In some embodiments, an intravenous administration can be an infusion occurring over a period of from about 10 minutes to about 4 hours. In some embodiments, an intravenous administration can be an infusion occurring over a period of from about 30 minutes to about 90 minutes.

In some embodiments, a dose can be administered weekly. In some embodiments, a dose can be administered bi-weekly. In some embodiments, a dose can be administered about every 2 weeks. In some embodiments, a dose can be administered about every 3 weeks. In some embodiments, a dose can be administered every four weeks.

In some embodiments, a total of from about 2 to about 10 doses are administered to a subject. In some embodiments, a total of 4 doses are administered. In some embodiments, a total of 5 doses are administered. In some embodiments, a total of 6 doses are administered. In some embodiments, a total of 7 doses are administered. In some embodiments, a total of 8 doses are administered. In some embodiments, a total of 9 doses are administered. In some embodiments, a total of 10 doses are administered. In some embodiments, a total of more than 10 doses are administered.

Pharmaceutical compositions containing a conjugate thereof can be administered in a unit dose. The term “unit dose” when used in reference to a pharmaceutical composition refers to physically discrete units suitable as unitary dosage for the subject, each unit containing a predetermined quantity of active material (e.g., a conjugate), calculated to produce the desired therapeutic effect in association with the required physiologically acceptable diluent, i.e., carrier, or vehicle.

In some embodiments, a conjugate, or a pharmaceutical composition of any of these, is administered with an immunosuppressive therapy. In some embodiments, provided is a method of improving treatment outcome in a subject receiving immunosuppressive therapy. The method generally includes administering an effective amount of an immunosuppressive therapy to the subject having an autoimmune disorder; and administering a therapeutically effective amount of a conjugate or a pharmaceutical composition thereof to the subject, wherein the conjugate specifically binds to target autoimmune cells; wherein the treatment outcome of the subject is improved, as compared to administration of the immunotherapy alone. In some embodiments, the conjugate thereof as described herein. In some embodiments, an improved treatment outcome is a decrease in disease progression, an alleviation of one or more symptoms, or the like.

The present invention is further illustrated by the following embodiments which should not be construed as limiting.

Embodiment 1. A Linker compound, or a stereoisomer or salt thereof, comprising:
(a) a Linker unit having from 1 to 4 attachment sites for a Drug unit and having one of the following structures (i) or (ii):

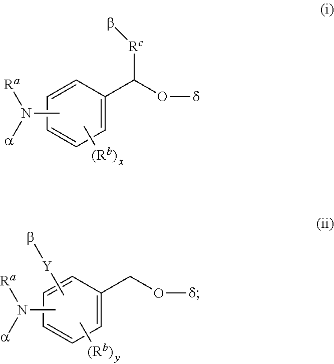

(b) at least one Polar group comprising a Polymer unit, optionally a Sugar unit, optionally a Carboxyl unit, and combinations thereof; and
(c) optionally a Stretcher group having an attachment site for a Targeting group;
   wherein:
α—is an attachment site to an enzyme-cleavable group;
β—is an attachment site to the at least one Polar group;
δ—is H, an attachment site to at least one of the Drug units, or an attachment site to a linking group attached to the at least one of the Drug units;
the Polymer unit comprises a polyamide, a polyether, or a combination thereof, wherein the polyether comprises a hydroxyl group, a polyhydroxyl group, a sugar group, a carboxyl group, or combinations thereof;
each R^{α} independently is H or C₁-C₆ alkyl;
each R^{b} independently is halo, C₁₋₆ alkyl, an attachment site to at least one of the Drug units, or an attachment site to at least one of the Polar groups;
x is 0, 1, 2, 3 or 4;
y is 0, 1, 2 or 3;
R^{c} is a bond, —C(O)—, —S(O)—, —SO₂—, C₁₋₆ alkylene, C₁₋₆ alkynylene, triazolyl or combinations thereof; and
Y is a bond, —O—, —S—, —N(R^{a})—, —C(O)—, —S(O)—, —SO₂—C₁-C₆ alkylene, C₁-C₆ alkenylene, C₁-C₆ alkynylene, a group containing triazolyl, or combinations thereof.

Embodiment 2. The Linker compound of Embodiment 1, wherein the Linker unit has one of the following structures (i-a), (ii-a), or (iii-a):

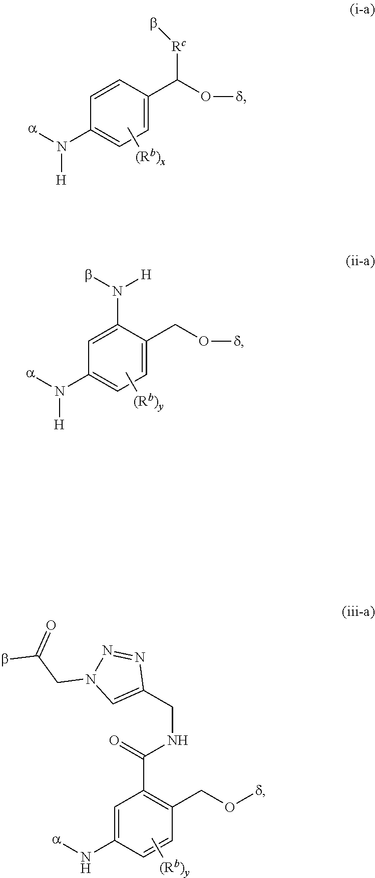

or a stereoisomer or salt thereof.

Embodiment 3. The Linker compound of Embodiment 1 or 2, wherein the Linker unit has one of the following structures (i-b), (i-c), (i-d), (i-e) or (i-f):

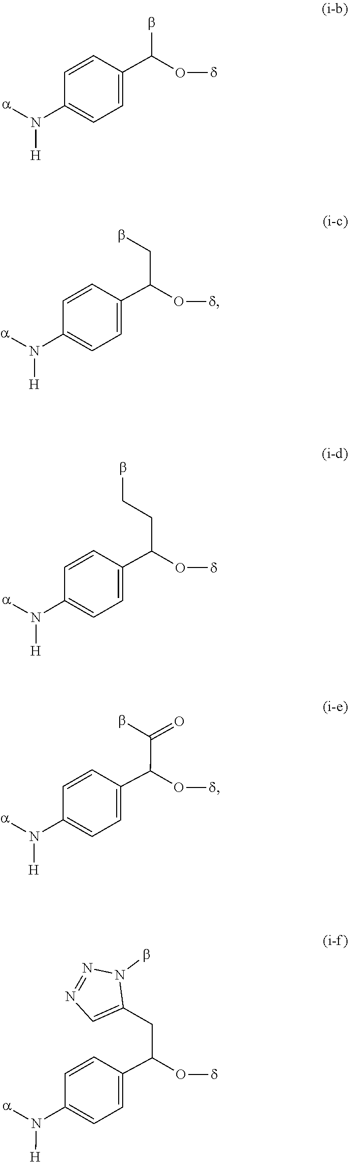

or a stereoisomer or salt thereof.

Embodiment 4. The Linker compound of Embodiment 1, wherein the Linker unit has the following structure (ii-b) or (iii-b):

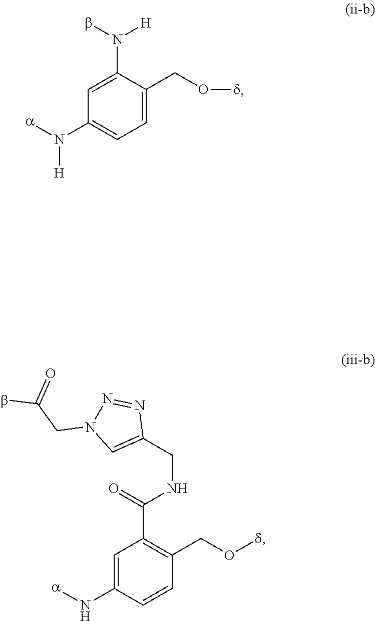

or a stereoisomer or salt thereof.

Embodiment 5. The Linker compound of any one of Embodiments 1-4, wherein the Polar group comprises at least one Sugar unit having the following formula:

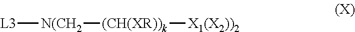

or a stereoisomer or salt thereof, wherein:
each X is independently selected from NH and O;
each R is independently selected from hydrogen, acetyl, a monosaccharide, a disaccharide, and a polysaccharide;
each X₁ is independently selected from CH₂ and C(O);
each X₂ is independently selected from H, OH and OR;
k is 1 to 10; and
L3 is a point of attachment to a remainder of the Polar group.

Embodiment 6. The Linker compound of Embodiment 5, wherein the at least one Sugar unit has one of the following structures (XII) or (XIII):

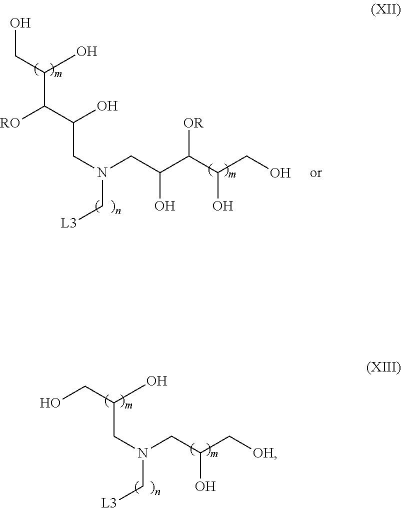

or a stereoisomer or salt thereof, wherein:
each R is independently selected from hydrogen, a monosaccharide, a disaccharide and a polysaccharide;
m is 1 to 8; and
n is 0 to 4.

Embodiment 7. The Linker compound of any one of Embodiments 1-6, comprising a Polar group having a formula selected from:

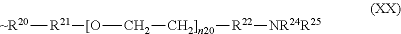

or a stereoisomer a salt thereof, wherein:
R²⁰ is an attachment group to site R^{b}, or to the enzyme-cleavable group;
R²¹ and R²² are each, independently, a bond or C₁-C₃ alkylene;
R²⁴ and R²⁵ are each independently selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; and —C(O)—R²⁸, where R²⁸ is a Sugar unit of formula (XII) or (XIII); or —NR²⁴R²⁵ together from a C₃-C₈ heterocycle; and
n20 is 2 to 26; or

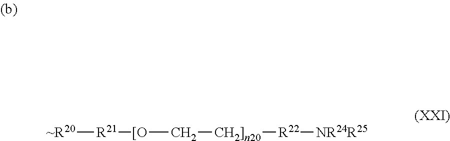

or a stereoisomer or salt thereof, wherein:
R²⁰ is an attachment group to site R^{b}, or to the enzyme-cleavable group;
R²¹ and R²² are each, independently, a bond or C₁-C₃ alkylene;
one of R²⁴ and R²⁵ is selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; and —C(O)—R²⁸, where R²⁸ is a Sugar unit of formula (XII) or (XIII); and the other of R²⁴ and R²⁵ is a polyethylene glycol, optionally having 1 to 24 ethylene glycol subunits; and
n20 is 2 to 26; or

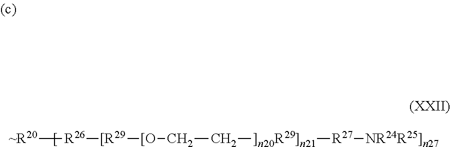

or a stereoisomer or salt thereof, wherein:
R²⁰ is an attachment group to site R^{b}, or to the enzyme-cleavable group;
R²⁶ and R²⁷ are each optional and are, independently, selected from a bond, C₁-C₁₂ alkylene, —NH—C₁-C₁₂ alkylene, —C₁-C₁₂ alkylene-NH—, —C₁-C₁₂ alkylene-N(CH₃)—, —C(O)—C₁-C₁₂ alkylene, —C₁-C₁₂ alkylene-C(O)—, —NH—C₁-C₁₂ alkylene-C(O)— and —C(O)—C₁-C₁₂ alkylene-NH—;
one of R²⁴ and R²⁵ is selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; —C(O)—R²⁸, where R²⁸ is a Sugar unit of formula (XII) or (XIII); and the other of R²⁴ and R²⁵ is selected from H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; and —C(O)—R²⁸, where R²⁸ is a Sugar unit of formula (XII) or (XIII); and polyethylene glycol, optionally having 1 to 24 ethylene glycol subunits; or —NR²⁴R²⁵ together from a C₃-C₈ heterocycle;
each R²⁹ is optional and independently selected from —C(O)—, —NH—, —C(O)—C₁-C₆ alkylene-, —NH—C₁-C₆ alkylene-, —C₁-C₆ alkylene-NH—, —C₁-C₆ alkylene-C(O)—, —NH(CO)—C₁-C₆alkylene-, —N(CH₃)—(CO)—C₁-C₆alkylene-, —NH(CO)NH—, and triazole;
n20 is 2 to 26;
n21 is 1 to 4; and
n27 is 1 to 4, or

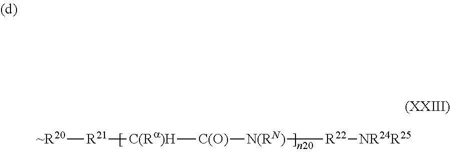

or a stereoisomer or salt thereof, wherein:
R²⁰ is an attachment group to site R^{b}, or to the enzyme-cleavable group;
R²¹ is a bond, C₁-C₃ alkylene,

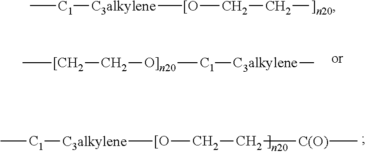

R²² is C₁-C₃ alkylene,

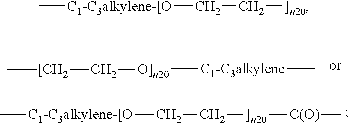

each R^{α} is independently H or —R²²—NR²⁴R²⁵;
each R^{N} is independently H, C₁-C₆ alkyl or —R²²—NR²⁴R²⁵
R²⁴ and R²⁵ are each independently selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; and —C(O)—R²⁸, where R²⁸ is a Sugar unit of formula (XII) or (XIII); or —NR²⁴R²⁵ together from a C₃-C₈ heterocycle; and
each n20 is independently 2 to 26, or

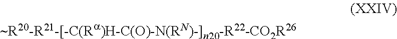

or a stereoisomer or salt thereof, wherein:
R²⁰ is an attachment group to site R^{b}, or to the enzyme-cleavable group;
R²¹ and R²² are each, independently, a bond, C₁-C₃ alkylene, or
—C₁-C₃alkylene[O—CH₂—CH₂-]ₙ₂₀;
each R^{α} is independently H or —R²²—NR²⁴R²⁵;
each R^{N} is independently H, C₁-C₆ alkyl or —R²²—NR²⁴R²⁵;
R²⁴ and R²⁵ are each independently selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; and —C(O)—R²⁸, where R²⁸ is a Sugar unit of formula (XII) or (XIII); or —NR²⁴R²⁵ together from a C₃-C₈ heterocycle;
R²⁶ is H or C₁-C₄ alkyl; and
each n20 is independently 2 to 26,
with the proviso that at least one R^{a} or R^{N} is —R²²—NR²⁴R²⁵; or

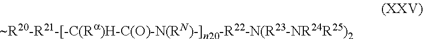

or a stereoisomer or salt thereof, wherein:
R²⁰ is an attachment group to site R^{b}, or to the enzyme-cleavable group;
R²¹ and R²² are each, independently, a bond, C₁-C₃ alkylene, or
—C₁-C₃alkylene-[O—CH₂—CH₂-]ₙ₂₀;
each R^{α} is independently H or —R²²—NR²⁴R²⁵;
each R^{N} is independently H or C₁-C₆ alkyl;
each R²³ is independently C₁-C₆ alkylene;
R²⁴ and R²⁵ are each independently selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; and —C(O)—R²⁸, where R²⁸ is a Sugar unit of formula (XII) or (XIII); or —NR²⁴R²⁵ together from a C₃-C₈ heterocycle; and
each n20 is independently 2 to 26.

Embodiment 8. The Linker compound of claim Embodiment 7, wherein both R²⁴ and R²⁵ are not H.

Embodiment 9. The Linker compound of Embodiment 7 or 8, wherein R²⁴ and R²⁵ are each independently selected from H and a polyhydroxyl group, provided that R²⁴ and R²⁵ are not both H.

Embodiment 10. The Linker compound of any one of Embodiments 7 to 9, wherein the polyhydroxyl group is a linear monosaccharide, optionally selected from a C6 or C5 sugar, a sugar acid and an amino sugar.

Embodiment 11. The Linker compound of Embodiment 10, wherein:
the C6 or C5 sugar is selected from glucose, ribose, galactose, mannose, arabinose, 2-deoxyglucose, glyceraldehyde, erythrose, threose, xylose, lyxose, allose, altrose, gulose, idose, talose, aldose, and ketose;
the sugar acid is selected from gluconic acid, aldonic acid, uronic acid and ulosonic acid; or
the amino sugar is selected from glucosamine, N-acetyl glucosamine, galactosamine, and N-acetyl galactosamine.

Embodiment 12. The Linker compound of any one of Embodiments 1 to 7, comprising a Polar group selected from the following, or a stereoisomer or salt thereof:

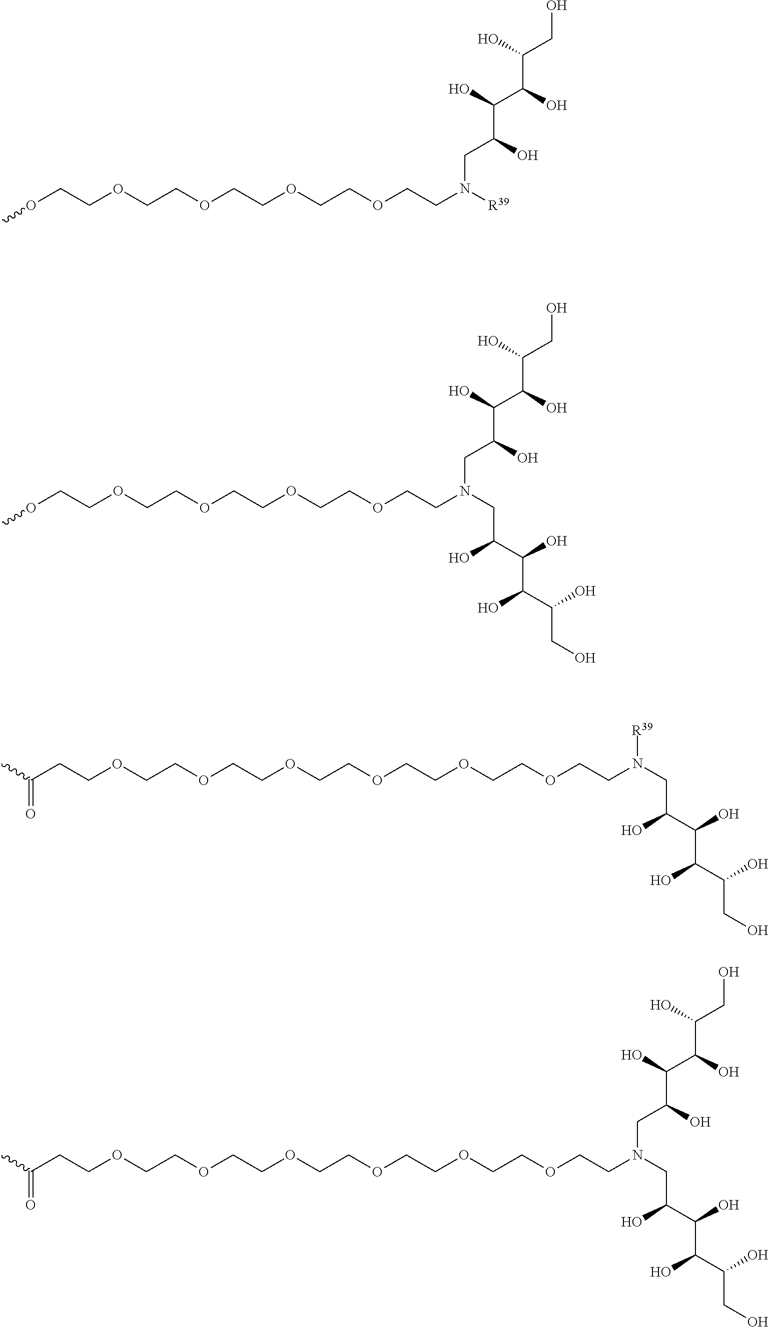

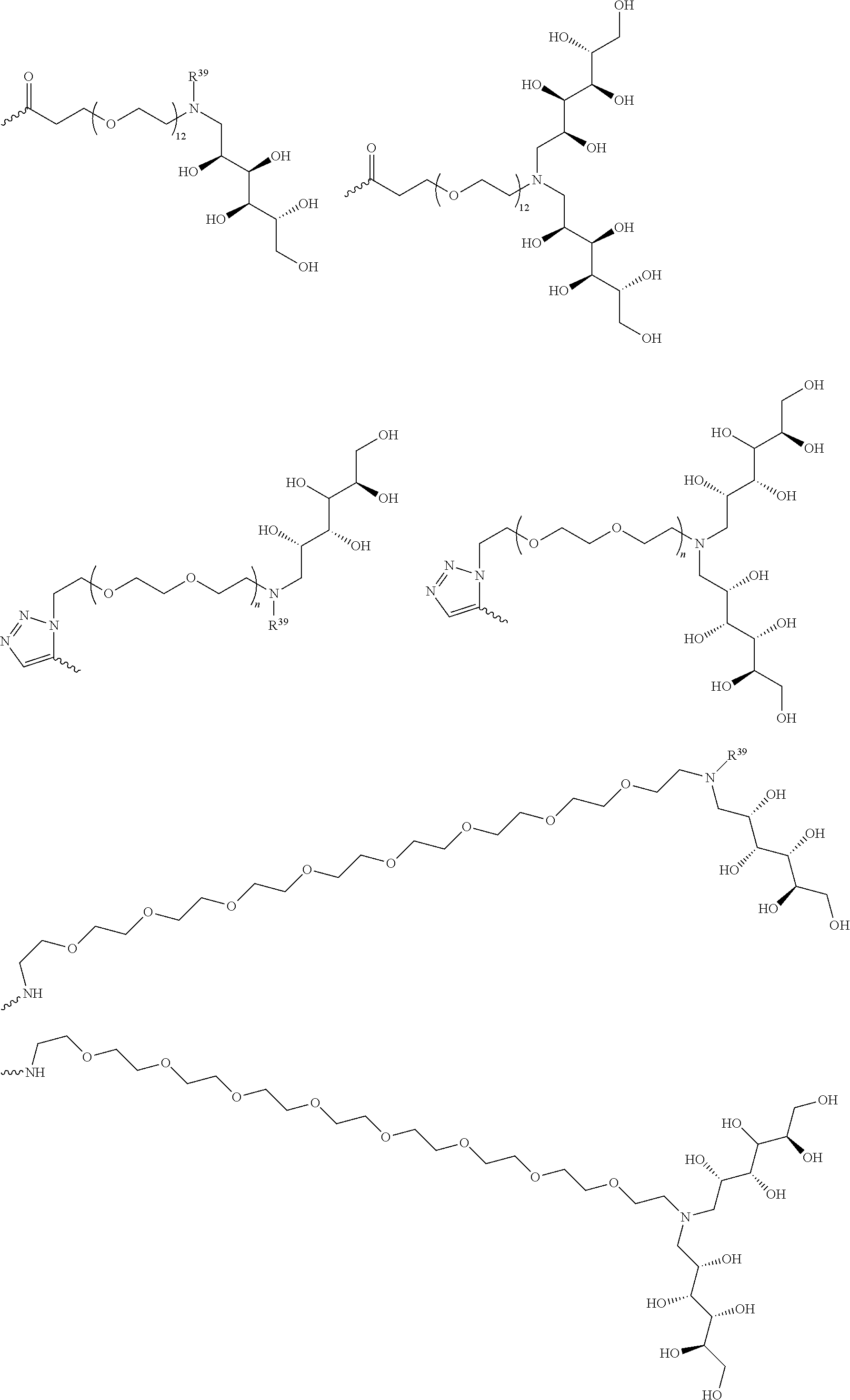

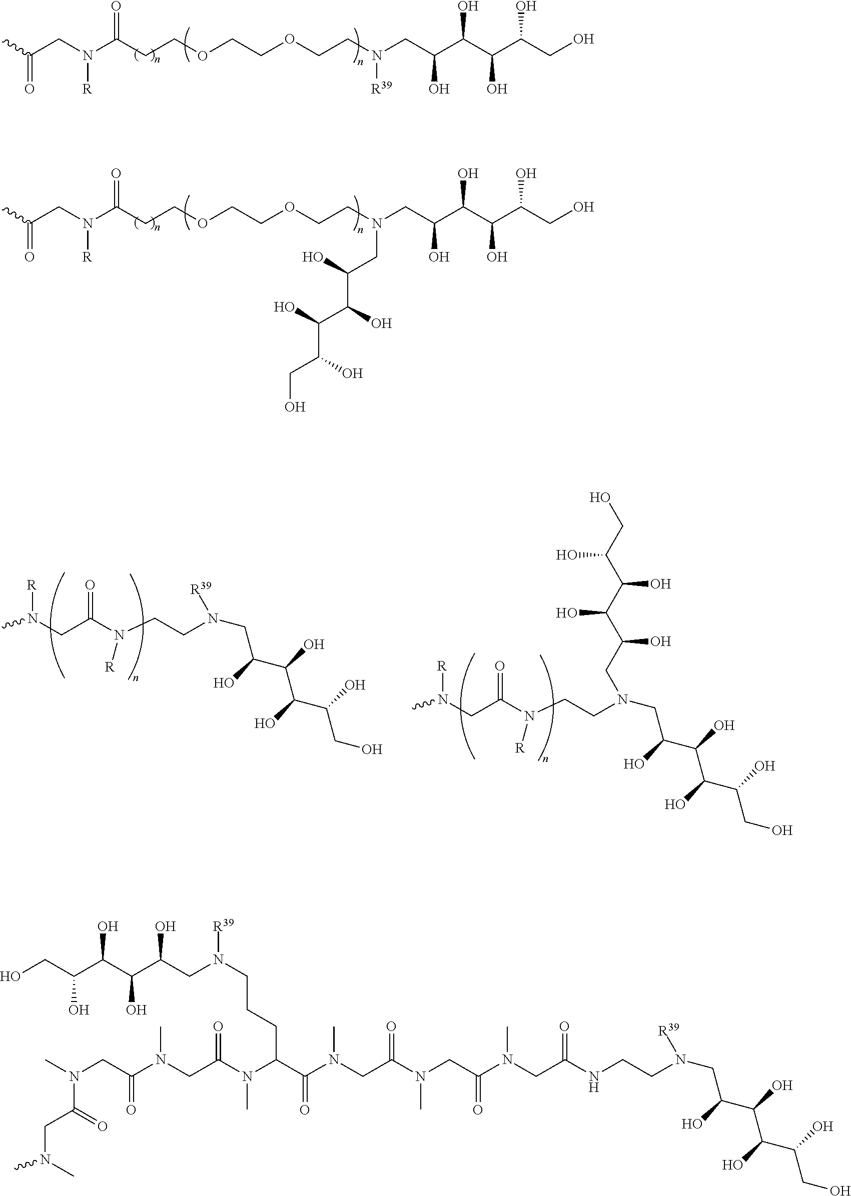

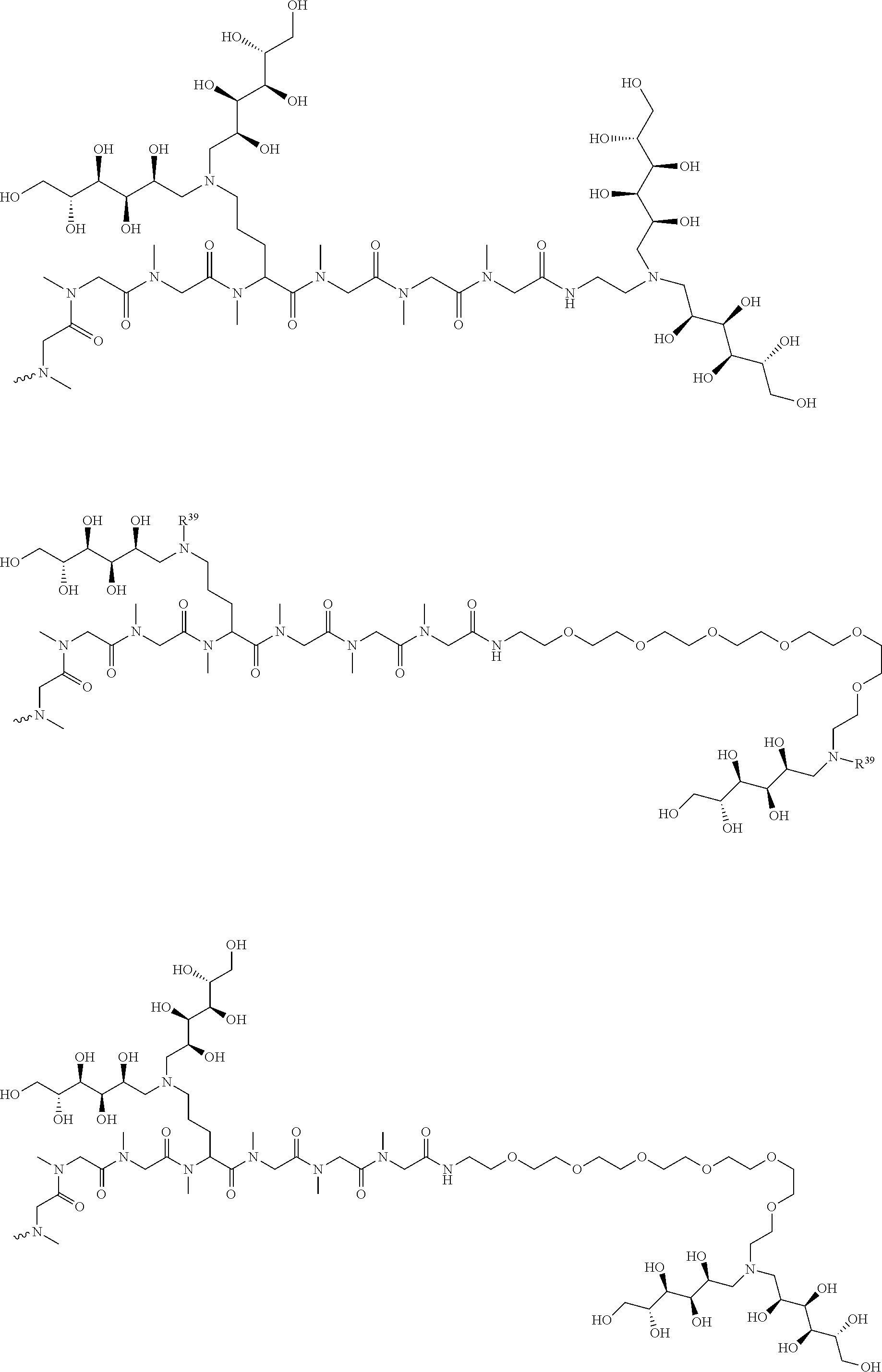

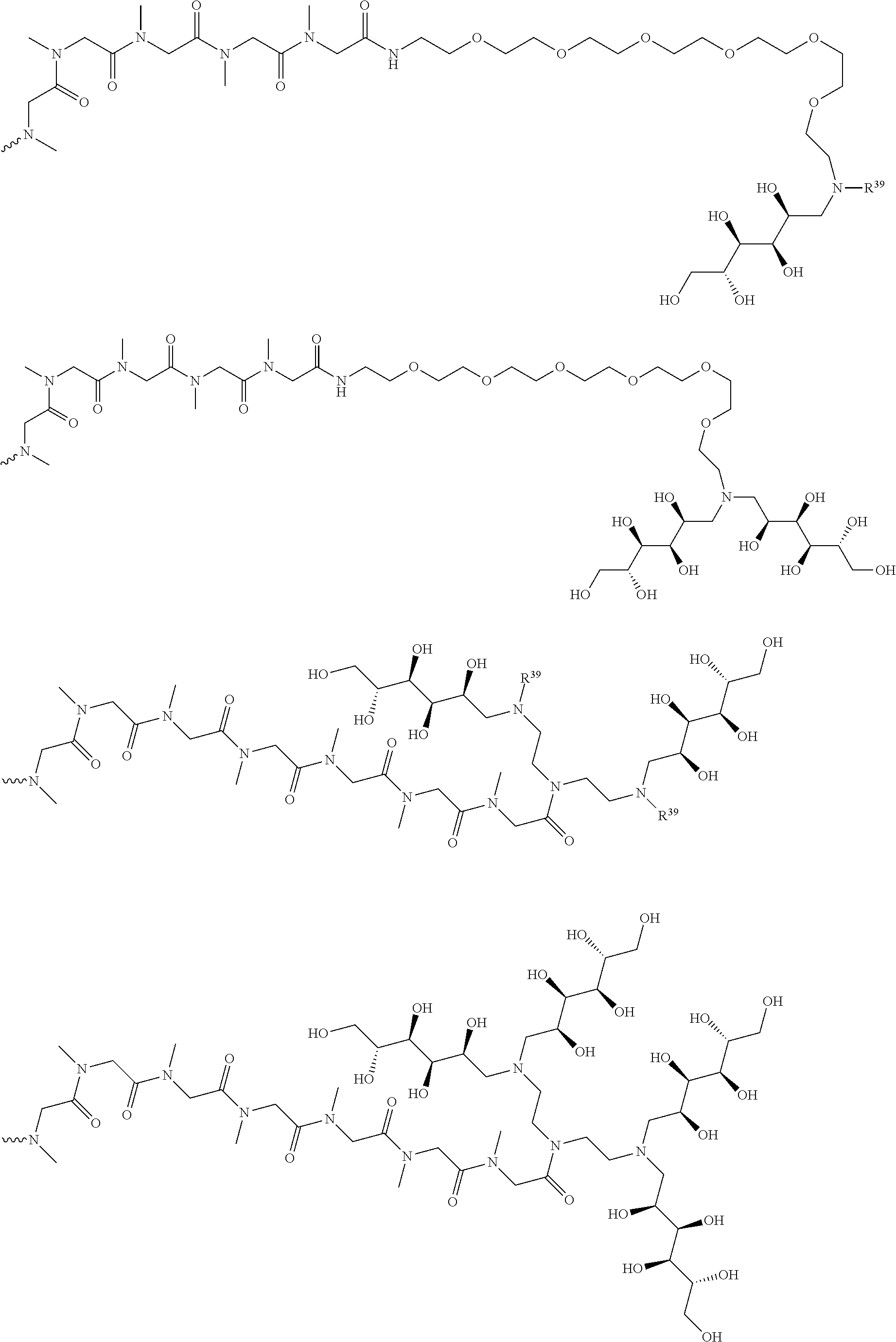

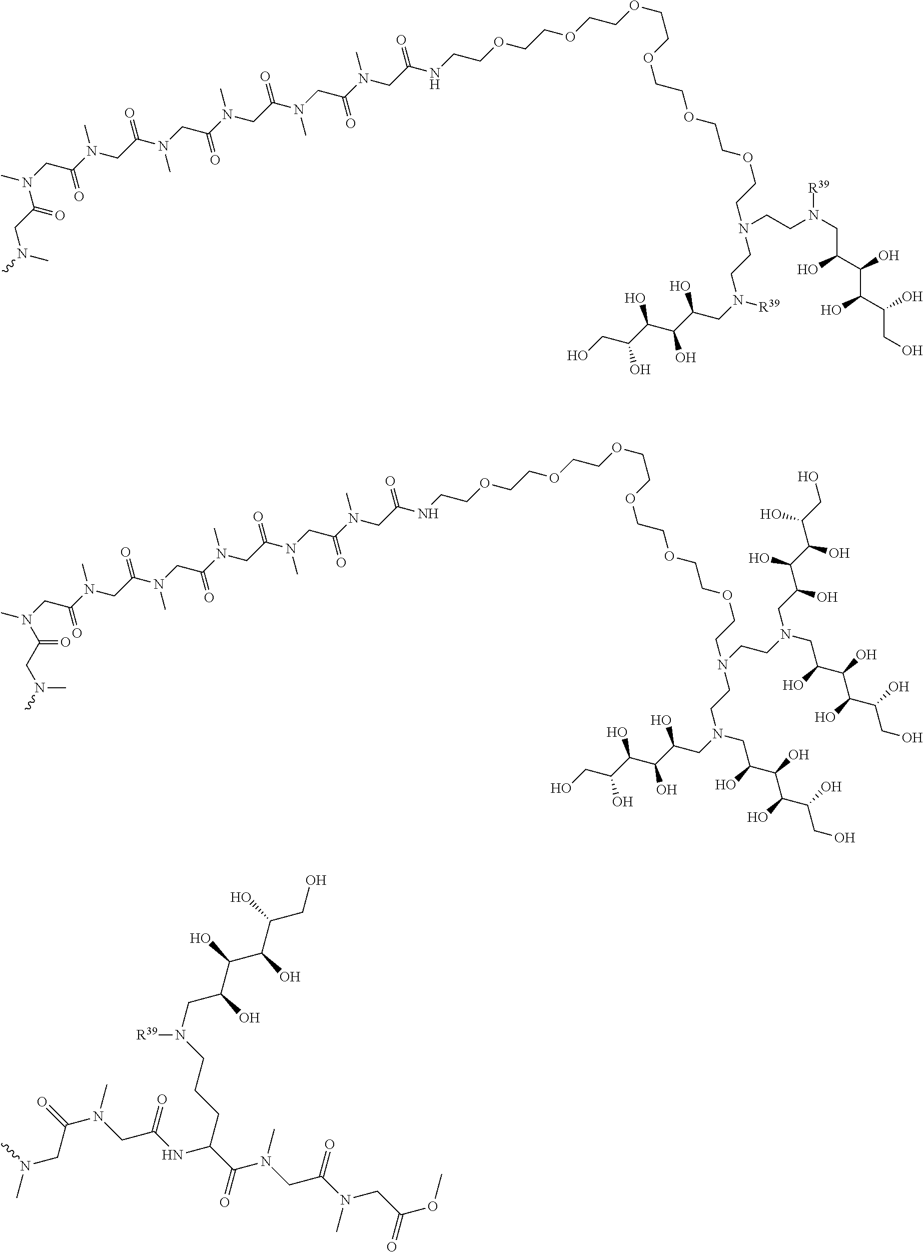

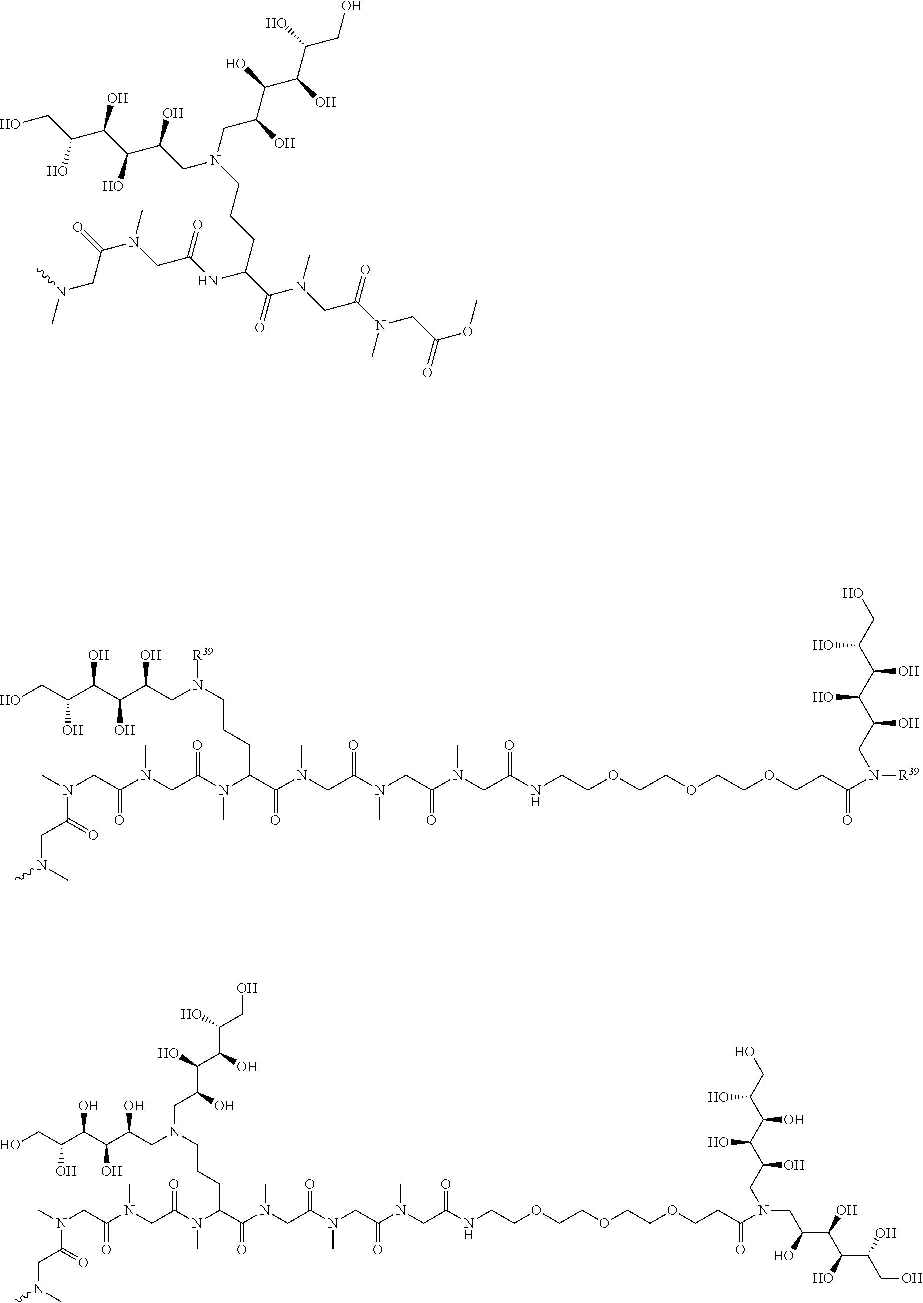

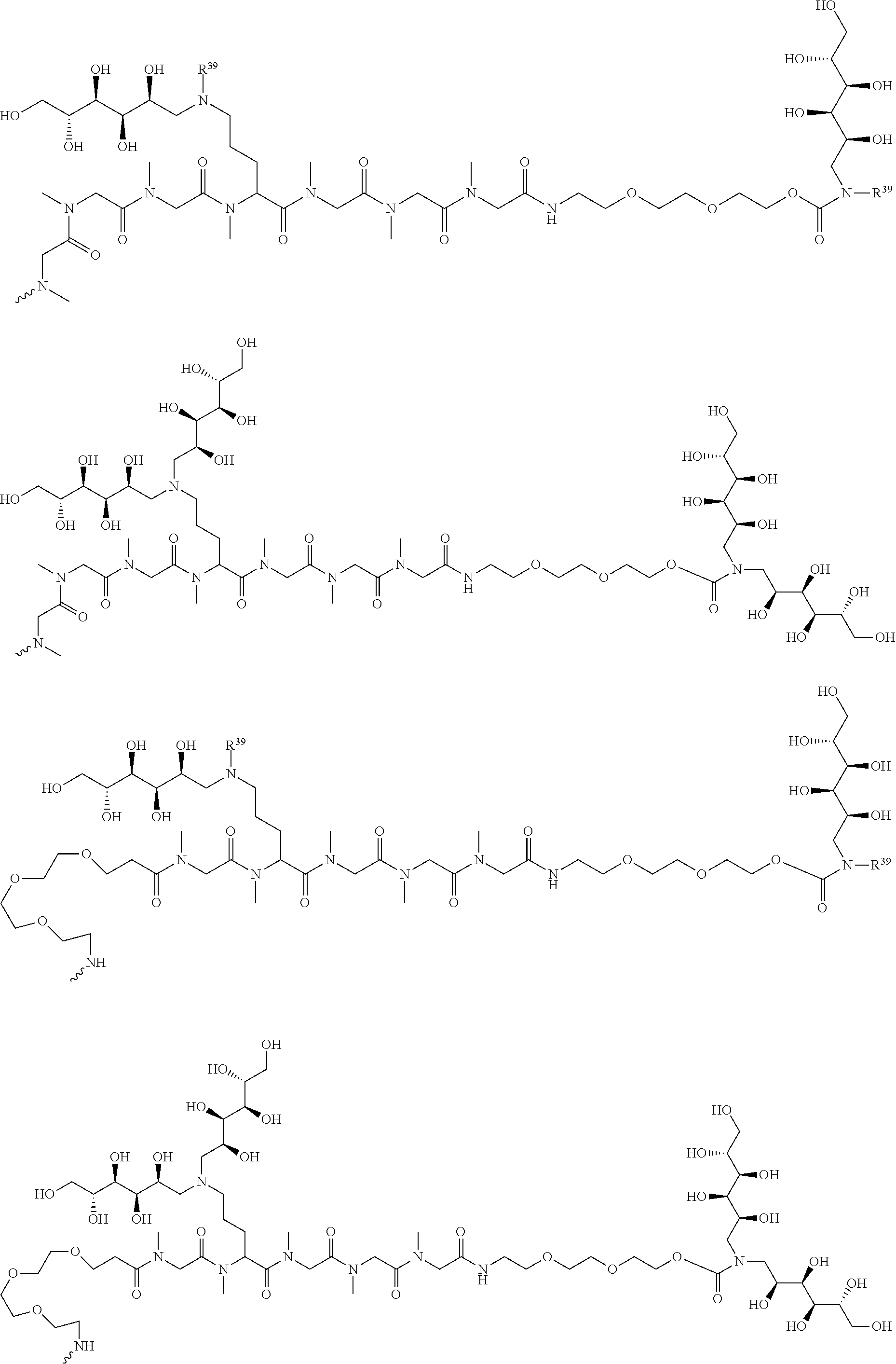

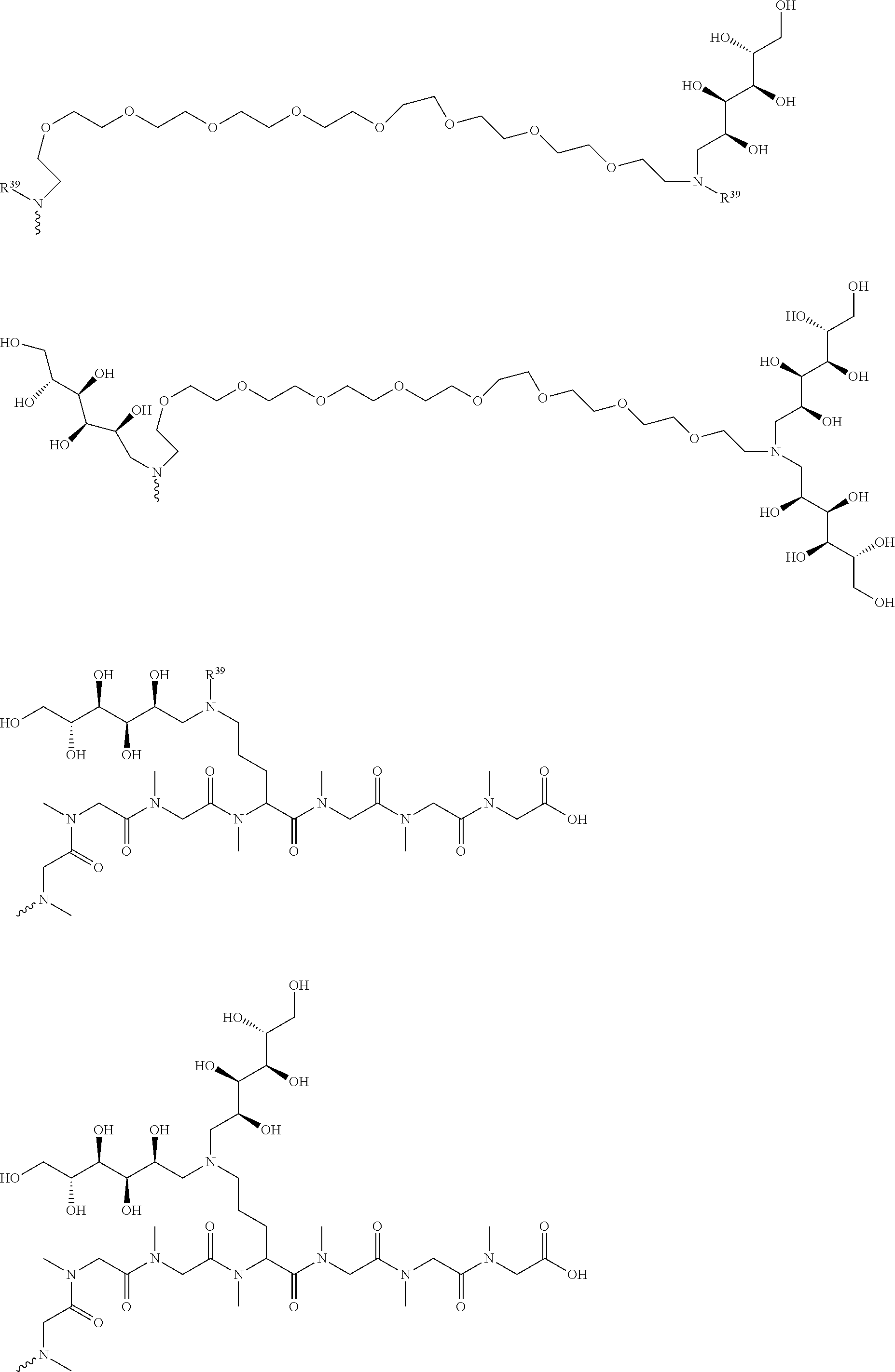

wherein each R is independently H or alkyl; each R³⁹ is independently selected from H, a linear monosaccharide and polyethylene glycol, optionally having from 1 to 24 ethylene glycol subunits; each n independently is 1-12; and the wavy line is an attachment site to R^{b}, or to the enzyme-cleavable group.

Embodiment 13. The Linker compound of Embodiment 7 or 8, wherein one of R²⁴ and R²⁵ is a linear monosaccharide and the other is a cyclic monosaccharide.

Embodiment 14. The Linker compound of any one of Embodiments 1-7, comprising a Polar group selected from the following, or a stereoisomer or salt thereof:

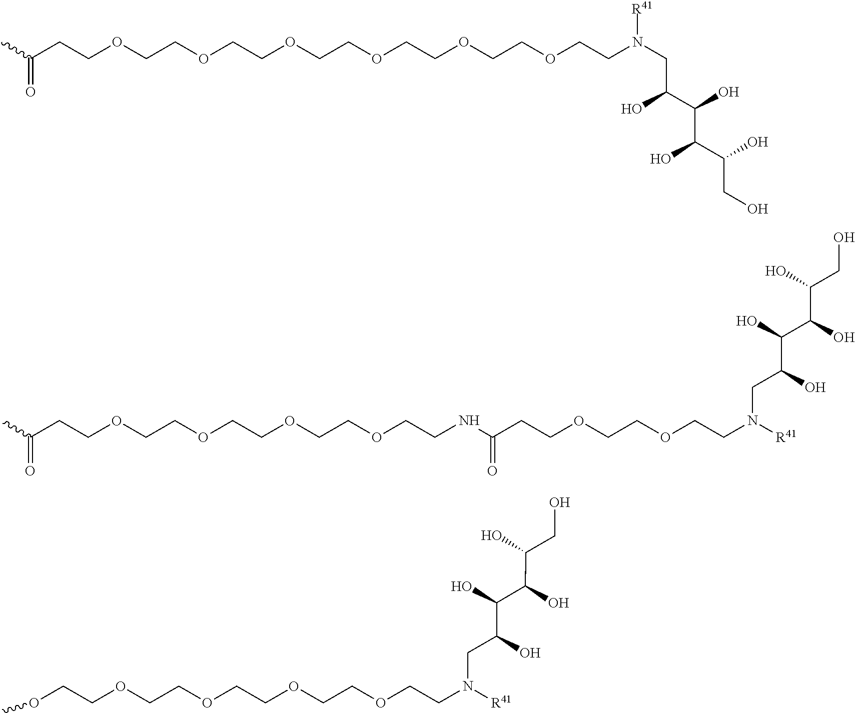

wherein R⁴¹ is a cyclic monosaccharide; and the wavy line is an attachment site to R^{b}, or to the enzyme-cleavable group.

Embodiment 15. The Linker compound of Embodiment 7 or 8, wherein R²⁴ and R²⁵ are independently a polyhydroxyl selected from a cyclic monosaccharide, disaccharide and polysaccharide.

Embodiment 16. The Linker compound of any one of Embodiments 1-7, comprising a Polar group selected from the following, or a stereoisomer or salt thereof:

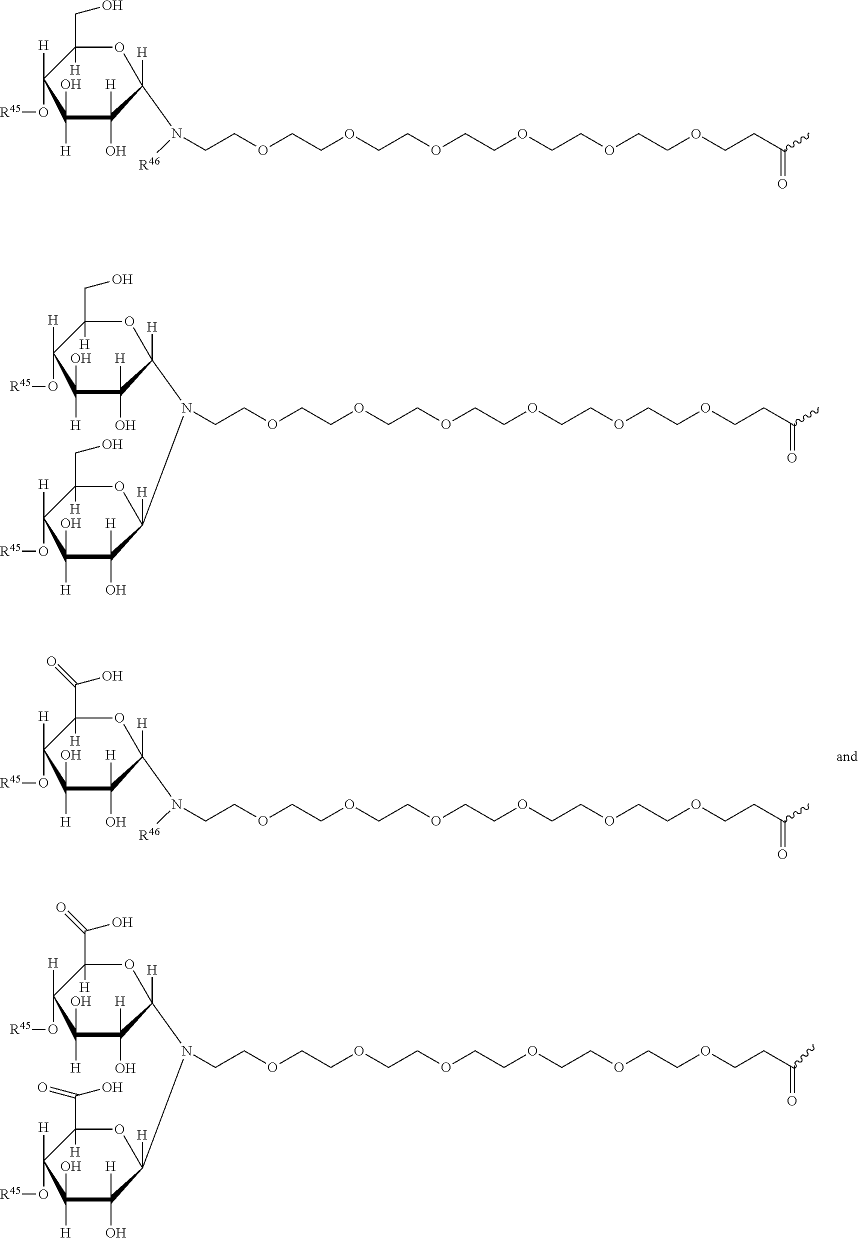

wherein each R⁴⁵ is selected from H and a monosaccharide, a disaccharide, or a polysaccharide; and R⁴⁶ is selected from a cyclic monosaccharide, disaccharide, or polysaccharide; and the wavy line is an attachment site to R^{b}, or to the enzyme-cleavable group.

Embodiment 17. The Linker compound of Embodiment 7 or 8, wherein R²⁴ and R²⁵ are independently selected from a linear monosaccharide and a substituted linear monosaccharide, wherein the substituted linear monosaccharide is substituted with a monosaccharide, a disaccharide or a polysaccharide.

Embodiment 18. The Linker compound of any one of Embodiments 1-7, comprising a Polar group selected from the following, or a stereoisomer or salt thereof:

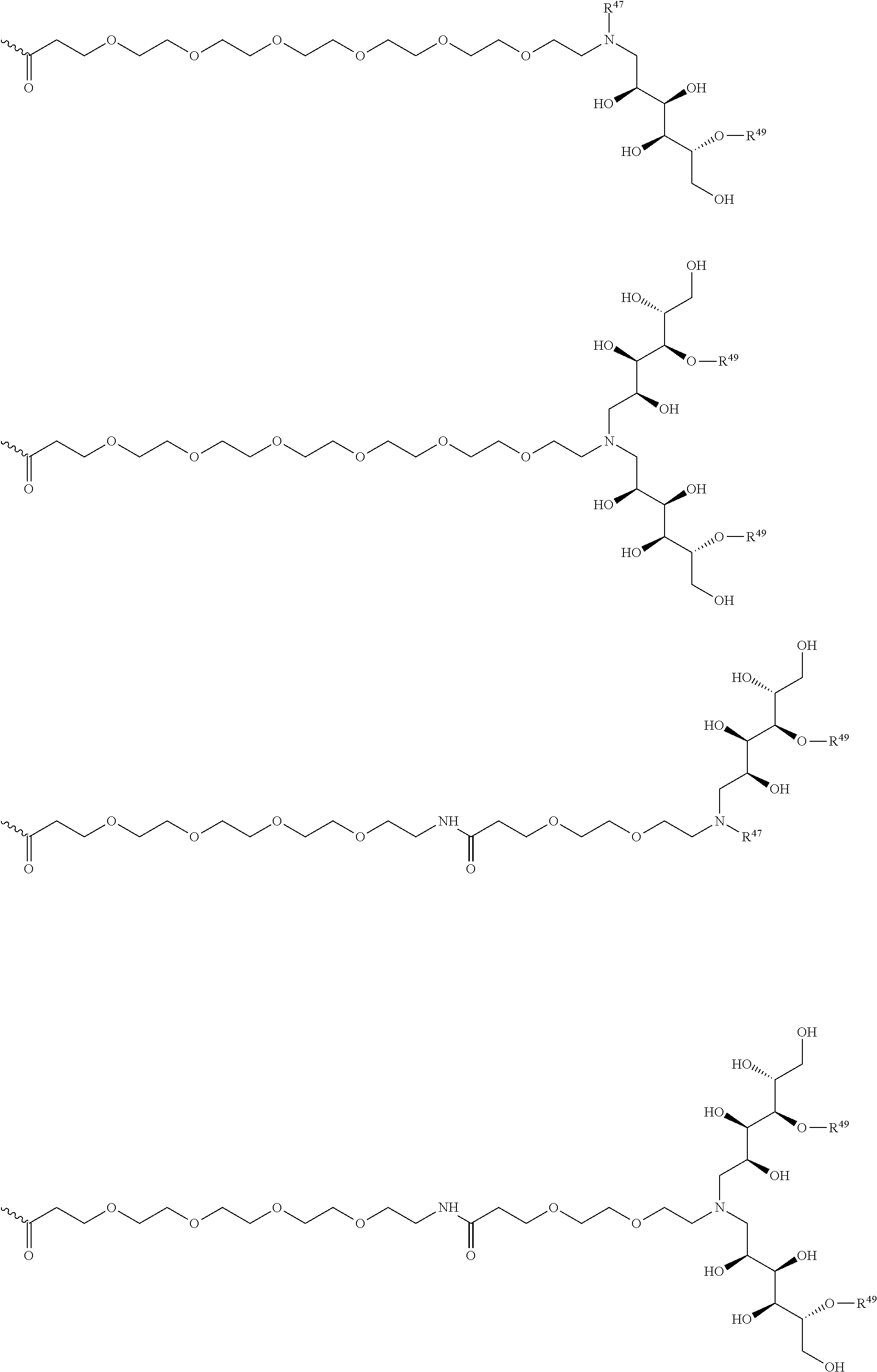

wherein R⁴⁷ is a linear monosaccharide; and each R⁴⁹ is selected from a monosaccharide, a disaccharide and a polysaccharide; and the wavy line is an attachment site to R^{b}, or to the enzyme-cleavable group.

Embodiment 19. The Linker compound of Embodiment 7 or 8, wherein R²⁴ and R²⁵ are independently selected from a linear monosaccharide and a substituted monosaccharide, wherein the substituted linear monosaccharide is substituted with one or more substituents selected from alkyl, O-alkyl, aryl, O-aryl, carboxyl, ester, or amide, and optionally further substituted with a monosaccharide, disaccharide or a polysaccharide.

Embodiment 20. The Linker compound of any one of Embodiments 1-7, comprising a Polar group selected from the following, or a stereoisomer or salt thereof:

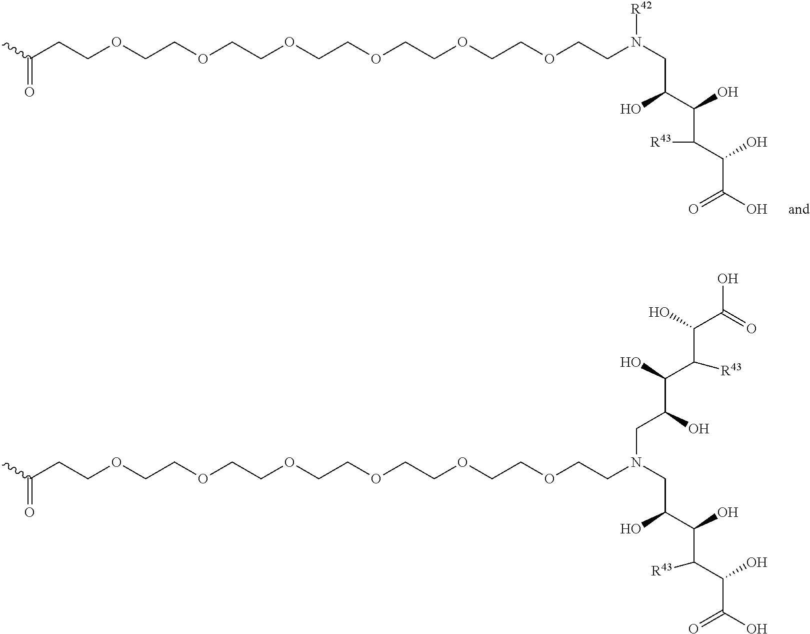

wherein each R⁴² is independently selected from a linear monosaccharide and a substituted linear monosaccharide; each R⁴³ is independently selected from alkyl, O-alkyl, aryl, O-aryl, carboxyl, ester, and amide; and the wavy line is an attachment site to R^{b}, or to the enzyme-cleavable group.

Embodiment 21. The Linker compound of any one of Embodiments 7 to 8, wherein one of R²⁴ and R²⁵ is a —C(O)-polyhydroxyl group or substituted —C(O)-polyhydroxyl group, and the other of R²⁴ and R²⁵ is a H, —C(O)-polyhydroxyl group, substituted —C(O)-polyhydroxyl group, polyhydroxyl group or substituted polyhydroxyl group; wherein the substituted —C(O)-polyhydroxyl group and polyhydroxyl group are substituted with a monosaccharide, a disaccharide, a polysaccharide, alkyl, —O-alkyl, aryl, carboxyl, ester, or amide.

Embodiment 22. The Linker compound of any one of Embodiments 1-21, comprising a Polar group selected from the following, or a stereoisomer or salt thereof:

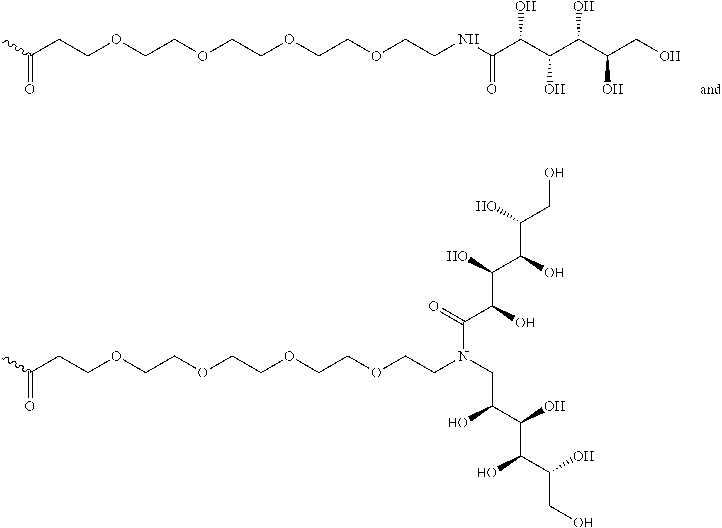

wherein the wavy line is an attachment site to R^{b}, or to the enzyme-cleavable group.

Embodiment 23. The Linker compound of Embodiment 7 or 8, wherein R²⁴ and R²⁵ are independently selected from a H, substituted —C₁-C₈ alkyl, substituted —C₁-C₄ alkyl or substituted —C₁-C₃ alkyl; provided that both R²⁴ and R²⁵ are not H; wherein substituted —C₁-C₈ alkyl, —C₁-C₄ alkyl, and —C₁-C₃ alkyl are substituted with hydroxyl and/or carboxyl.

Embodiment 24. The Linker compound of Embodiments 1-7, comprising a Polar group selected from the following, or a stereoisomer or salt thereof:

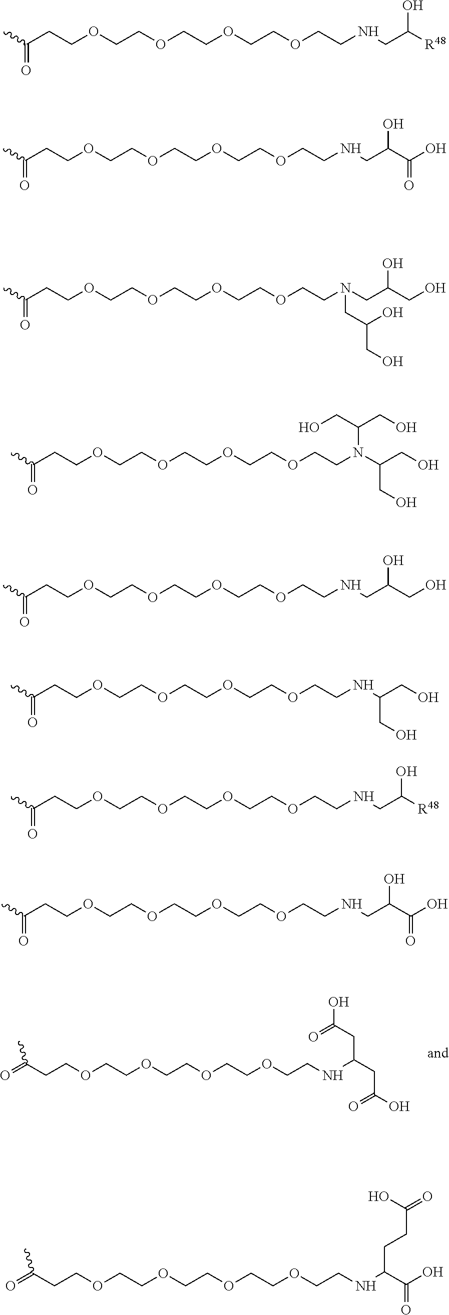

wherein R⁴⁸ is selected from H, OH, CH₂OH, COOH or —C₁-C₆ alkyl substituted with hydroxyl or carboxyl; and the wavy line is an attachment site to R^{b}, or to the enzyme-cleavable group.

Embodiment 25. The Linker compound of Embodiment 7 or 8, wherein one of R²⁴ and R²⁵ is selected from H, substituted —C(O)—C₁-C₈ alkyl, substituted —C(O)—C₁-C₄ alkyl, and substituted —C(O)—C₁-C₃ alkyl and the other of R²⁴ and R²⁵ is selected from substituted —C(O)—C₁-C₈ alkyl, substituted —C(O)—C₁-C₄ alkyl, substituted —C(O)—C₁-C₃ alkyl, substituted —C₁-C₈ alkyl, substituted —C₁-C₄ alkyl, and substituted —C₁-C₃ alkyl, wherein substituted —C(O)—C₁-C₈ alkyl, substituted —C(O)—C₁-C₄ alkyl, substituted —C(O)—C₁-C₃ alkyl, substituted —C₁-C₈ alkyl, —C₁-C₄ alkyl and —C₁-C₃ alkyl are substituted with hydroxyl and/or carboxyl.

Embodiment 26. The Linker compound of any one of Embodiments 1-7, comprising a Polar group selected from the following, or a stereoisomer or salt thereof:

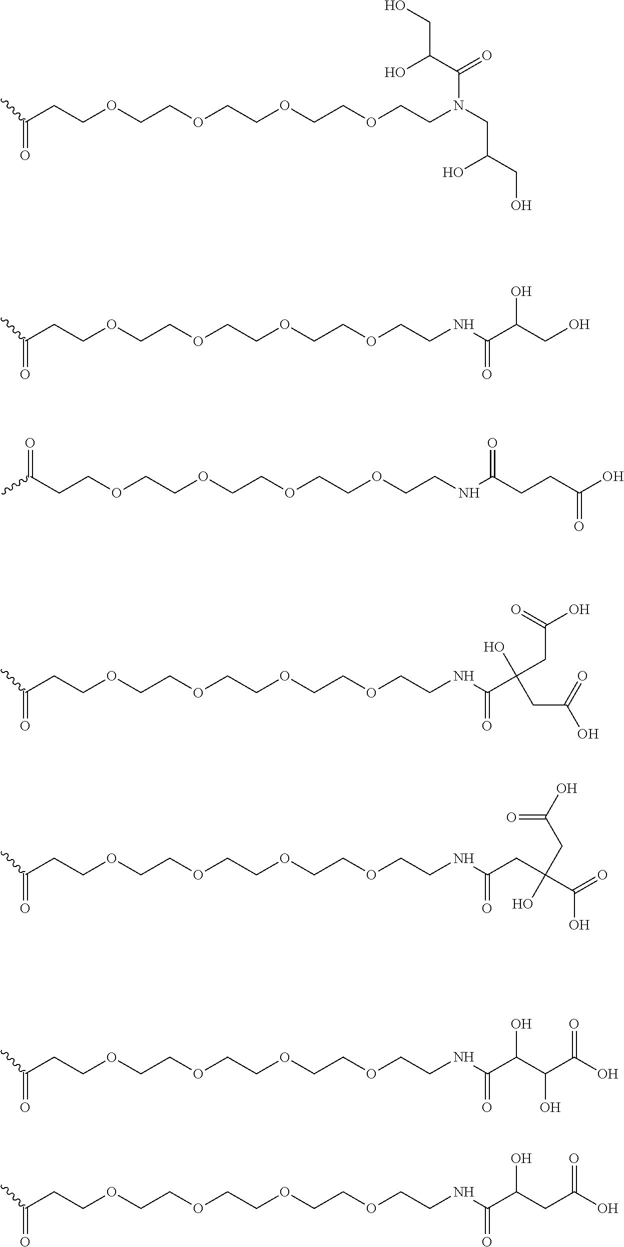

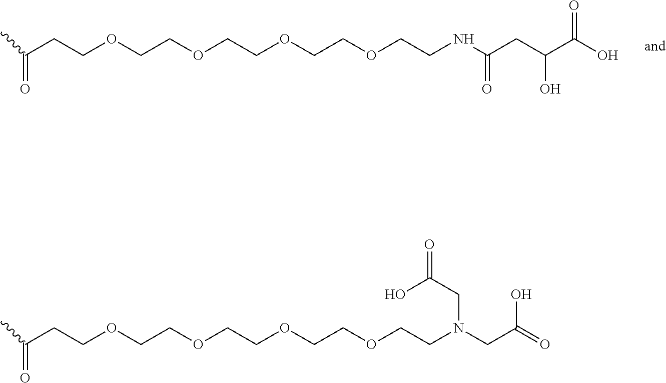

wherein the wavy line is an attachment site to R^{b}, or to the enzyme-cleavable group.

Embodiment 27. The Linker compound of any one of Embodiments 6 to 8, wherein R²⁴ and R²⁵ are selected from H and optionally substituted aryl; provided that both R²⁴ and R²⁵ are not H.

Embodiment 28. The Linker compound of any one of Embodiments 1-7, comprising a Polar group selected from the following, or a stereoisomer or salt thereof:

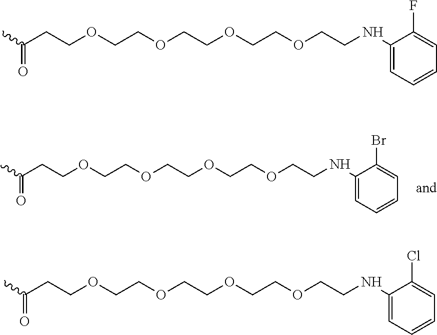

wherein the wavy line is an attachment site to R^{b}, or to the enzyme-cleavable group.

Embodiment 29. The Linker compound of Embodiment 7 or 8, wherein R²⁴ and R²⁵ together form an optionally substituted C₃-Cₑ heterocycle or heteroaryl.

Embodiment 30. The Linker compound of any one of Embodiments 1-7, comprising a Polar group having the following structure:

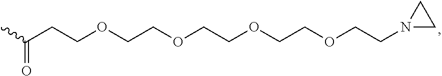

or a stereoisomer or salt thereof, wherein the wavy line is an attachment site to R^{b}, or to the enzyme-cleavable group.

Embodiment 31. The Linker compound of Embodiment 7 or 8, wherein R²⁴ and R²⁵ are independently selected from H and a chelator, wherein the chelator is optionally attached to the nitrogen of —NR²⁴R²⁵ by an alkylene, arylene, carbocyclo, heteroarylene or heterocarbocylo; provided that both R²⁴ and R²⁵ are not H.

Embodiment 32. The Linker compound of Embodiment 31, wherein the chelator is selected from ethylenediaminetetraacetic acid (EDTA), diethylenetriaminepentaacetic acid (DTPA), triethylenetetraminehexaacetic acid (TTHA), benzyl-DTPA, 1,4,7,10-tetraazacyclododecane-N,N′,N″,N′″-tetraacetic acid (DOTA), benzyl-DOTA, 1,4,7-triazacyclononane-N,N′,N″-triacetic acid (NOTA), benzyl-NOTA, 1,4,8,11-tetraazacyclotetradecane-1,4,8,11-tetraacetic acid (TETA) and N,N′-dialkyl substituted piperazine.

Embodiment 33. The Linker compound of any one of Embodiments 1-7, comprising a Polar group selected from the following, or a stereoisomer or salt thereof:

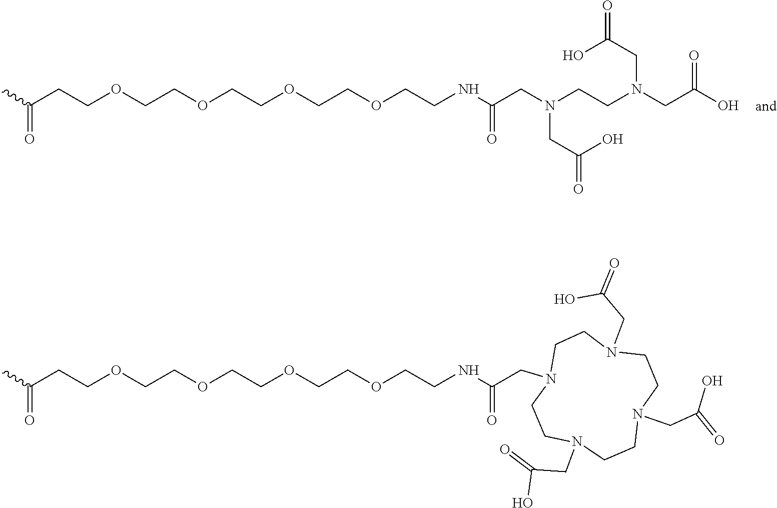

wherein the wavy line is an attachment site to R^{b}, or to the enzyme-cleavable group.

Embodiment 34. The Linker compound of any one of Embodiments 6 to 21, wherein each monosaccharide is independently selected from:
a C5 or C6 sugar selected from glucose, ribose, galactose, mannose, arabinose, 2-deoxyglucose, glyceraldehyde, erythrose, threose, xylose, lyxose, allose, altrose, gulose, idose talose, aldose, ketose, glucosamine, N-acetyl glucosamine, galactosamine, and N-acetyl galactosamine;
a sugar acid selected from gluconic acid, aldonic acid, uronic acid and ulosonic acid; or
an amino sugar is selected from glucosamine, N-acetyl glucosamine, galactosamine, and N-acetyl galactosamine.

Embodiment 35. The Linker compound of any one of Embodiments 1 to 34, wherein the attachment site is formed from a functional group of a precursor compound of the Polar group, said functional group selected from halo, aldehyde, carboxyl, amino, alkynyl, azido, hydroxyl, carbonyl, carbamate, thiol, urea, thiocarbamate, thiourea, sulfonamide, acyl sulfonamide, alkyl sulfonate, triazole, azadibenzocyclooctyne, hydrazine, carbonylalkylheteroaryl, and protected forms thereof.

Embodiment 36. The Linker compound of any one of Embodiments 1 to 35, comprising a Polar group having a formula selected from the following:

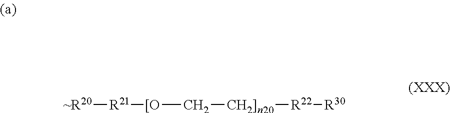

or a stereoisomer or salt thereof, wherein:
R²⁰ is an attachment group to site R^{b}, or to the enzyme-cleavable group;
R²¹ and R²² are each independently, a bond or C₁-C₃ alkylene groups;
R³⁰ is selected from an optionally substituted C₃-C₁₀ carbocycle; thiourea; optionally substituted thiourea; urea; optionally substituted urea; sulfamide; alkyl sulfamide; acyl sulfamide, optionally substituted alkyl sulfamide; optionally substituted acyl sulfamide; sulfonamide; optionally substituted sulfonamide; guanidine, including alkyl and aryl guanidine; phosphoramide; or optionally substituted phosphoramide; or R³⁰ is selected from azido, alkynyl, substituted alkynyl, —NH—C(O)-alkynyl, —NH—C(O)— alkynyl-R⁶⁵; cyclooctyne; —NH-cyclooctyne, —NH—C(O)-cyclooctyne, or —NH-(cyclooctyne)₂; wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and
n20 is 2 to 26;

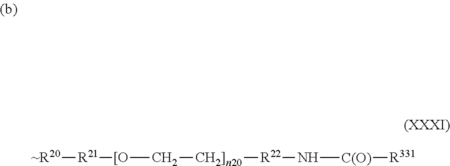

or a stereoisomer or salt thereof, wherein:
R²⁰ is an attachment group to site R^{b}, or to the enzyme-cleavable group;
R²¹ and R²² are each, independently, a bond or C₁-C₃ alkylene groups;
R³¹ is a branched polyethylene glycol chain, each branch having 1 to 26 ethylene glycol subunits and each branch having an R³⁵ at its terminus;
R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and
n20 is 2 to 26;

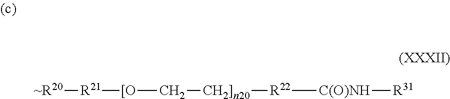

or a stereoisomer or salt thereof, wherein:
R²⁰ is an attachment group to site R^{b}, or to the enzyme-cleavable group;
R²¹ and R²² are each, independently, a bond or C₁-C₃ alkylene groups;
R³¹ is a branched polyethylene glycol chain, each branch, independently, having 1 to 26 ethylene glycol subunits and each branch having an R³⁵ at its terminus; R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle and optionally substituted heteroaryl; and
n20 is 2 to 26;

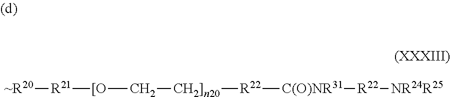

or a stereoisomer or salt thereof, wherein:
R²⁰ is an attachment group to site R^{b}, or to the enzyme-cleavable group;
R³¹ is H or R²²—NR²⁴R²⁵;
R²¹ and R²² are each, independently, a bond or C₁-C₃ alkylene groups;
R²⁴ and R²⁵ are each independently selected from a H; polyhydroxyl group; substituted polyhydroxyl group; —C(O)-polyhydroxyl group; substituted —C(O)-polyhydroxyl group, provided that R²⁴ and R²⁵ are not both H; and
n20 is 2 to 26;

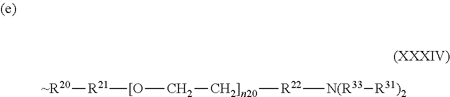

or a stereoisomer or salt thereof, wherein:
R²⁰ is an attachment group to site R^{b}, or to the enzyme-cleavable group;
R²¹ and R²² are each, independently, a bond or C₁-C₃ alkylene groups;
R³¹ is a branched polyethylene glycol chain, each branch having 1 to 26 ethylene glycol subunits and each branch having an R³⁵ at its terminus;
R³³ is C₁-C₃ alkylene, C₁-C₃ alkylene-C(O), —C(O)—C₁-C₃ alkylene, or —C(O)—C₁-C₃ alkylene-C(O);
R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and
n20 is 2 to 26;

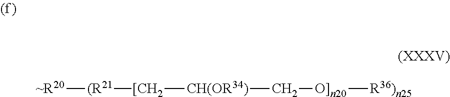

or a stereoisomer or salt thereof, wherein:
R²⁰ is an attachment group to site R^{b}, or to the enzyme-cleavable group;
each R²¹ is independently a bond, —O— or C₁-C₃ alkylene group;
each R³⁴ is independently H, —[CH₂—CH(OH)—CH₂—O]ₙ₂₀—R³⁶, —C(O)—NR²⁴R²⁵ or —C(O)N(R^{N})—C₁-C₆alkylene-NR²⁴R²⁵
R^{N} is H or C₁-C₄alkyl;
R²⁴ and R²⁵ are each independently selected from a H; polyhydroxyl group; or substituted polyhydroxyl group, provided that both R²⁴ and R²⁵ are not H;
each R³⁶ is independently H, C₁-C₆alkylene-C(OH)H—NR⁴⁴R⁴⁵, C₁-C₆alkylene-C(OH)H—C₁-C₆alkylene-NR⁴⁴R⁴⁵, —C(O)—NR²⁴R²⁵, —C(O)N(R^{N})—C₁-C₆alkylene-NR²⁴R²⁵, C₁-C₆alkylene-C(O)NR²⁴R²⁵ or C₁-C₆alkylene-CO₂R³⁷;
each R³⁷ is independently H or C₁-C₆ alkyl;
R⁴⁴ and R⁴⁵ are each independently selected from a H; polyhydroxyl group; substituted polyhydroxyl group; —C(O)-polyhydroxyl group; substituted —C(O)-polyhydroxyl group; provided that both R⁴⁴ and R⁴⁵ are not H;
each n20 is independently 1 to 26; and
n25 is 1 or 2;

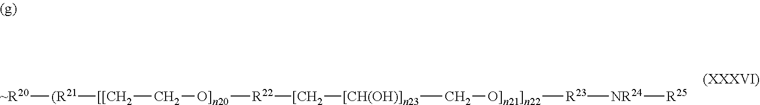

or a stereoisomer or salt thereof, wherein:
R²⁰ is an attachment group to site R^{b}, or to the enzyme-cleavable group;
R²¹, R²² and R²³ are each independently a bond or C₁-C₃ alkylene group;
R²⁴ and R²⁵ are each independently selected from a H; polyhydroxyl group; substituted polyhydroxyl group; —C(O)-polyhydroxyl group; substituted —C(O)-polyhydroxyl group, provided that R²⁴ and R²⁵ are not both H;
each n20 is independently 0 to 26, and each n21 is independently 0 to 26, with the proviso that at least one of n20 or n21 is 2 to 26;
n22 is 1 to 5;
each n23 is independently 1 or 2;

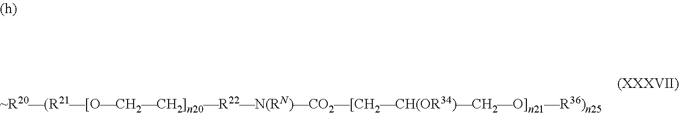

or a stereoisomer or salt thereof, wherein:
R²⁰ is an attachment group to site R^{b}, or to the enzyme-cleavable group;
R²¹ and R²² are each independently a bond or C₁-C₃ alkylene groups;
R^{N} is H or C₁-C₄alkyl;
R²⁴ and R²⁵ are each independently selected from a H; polyhydroxyl group; or substituted polyhydroxyl group, provided that both R²⁴ and R²⁵ are not H;
each R³⁴ is independently H, —[CH₂—CH(OH)—CH₂—O]ₙ₂₀—R³⁶ or —C(O)N(R^{N})—C₁-C₆alkylene-NR²⁴R²⁵;
each R³⁶ is independently H, C₁-C₆alkylene-C(OH)H—NR⁴⁴R⁴⁵, C₁-C₆alkylene-C(OH)H—C₁-C₆alkylene-NR⁴⁴R⁴⁵, —C(O)N(R^{N})_C₁-C₆alkylene-NR²⁴R²⁵, C₁-C₆alkylene-C(O)NR²⁴R²⁵ or C₁-C₆alkylene-CO₂R³⁷;
each R³⁷ is independently H or C₁-C₆ alkyl;
R⁴⁴ and R⁴⁵ are each independently selected from a H; polyhydroxyl group; substituted polyhydroxyl group; —C(O)-polyhydroxyl group; substituted —C(O)-polyhydroxyl group; provided that both R⁴⁴ and R⁴⁵ are not H;
n20 is 2 to 26;
n21 is 1 to 26; and
n25 is 1 or 2;

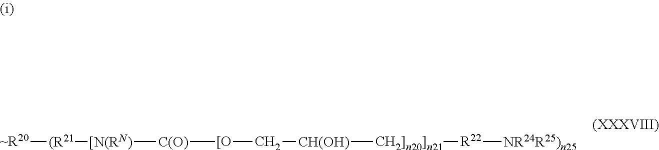

or a stereoisomer or salt thereof, wherein:
R²⁰ is an attachment group to site R^{b}, or to the enzyme-cleavable group;
R²¹ and R²² are each independently bond or C₁-C₃ alkylene groups;
R^{N} is H or C₁-C₄alkyl;
R²⁴ and R²⁵ are each independently selected from a H; polyhydroxyl group; or substituted polyhydroxyl group, provided that R²⁴ and R²⁵ are not both H;
n20 is 2 to 26;
n21 is 1 to 4; and
n25 is 1, 2 or 3;

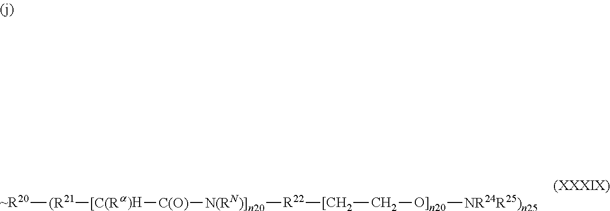

or a stereoisomer or salt thereof, wherein:
R²⁰ is an attachment group to site R^{b}, or to the enzyme-cleavable group;
R²¹ and R²² are each, independently, a bond, C₁-C₃ alkylene, —C₁-C₃alkylene-[O—CH₂—CH₂-]ₙ₂₀, —[CH₂—CH₂—O]ₙ₂₀—C₁-C₃alkylene- or —C₁-C₃alkylene-[O—CH₂—CH₂-]ₙ₂₀-C(O)—;
each R^{α} is independently H or —R²²NR²⁴R²⁵;
each R^{N} is independently H, C₁-C₆ alkyl or —R²²—NR²⁴R²⁵;
R²⁴ and R²⁵ are each independently selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; —C(O)—R²⁸, wherein R²⁸ is a Sugar unit of formula (XII) or (XIII); or —NR²⁴R²⁵ together from a C₃-C₈ heterocycle), provided that R²⁴ and R²⁵ are not both H;
each n20 is independently 0 to 26, with the proviso that at least one n20 is 2 to 26; and
n25 is 1 or 2; or

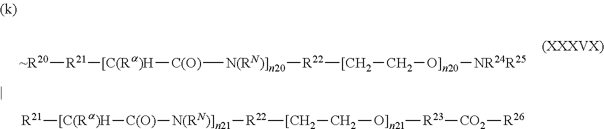

or a stereoisomer or salt thereof, wherein:
R²⁰ is an attachment group to site R^{b}, or to the enzyme-cleavable group;
R²¹, R²² and R²³ are each, independently, a bond, C₁-C₃ alkylene, —C₁-C₃alkylene-[O—CH₂—CH₂-]ₙ₂₀, —[CH₂—CH₂—O]ₙ₂₀—C₁-C₃alkylene- or —C₁-C₃alkylene-[O—CH₂—CH₂-]ₙ₂₀-C(O)—;
each R^{α} is independently H or —R²²—NR²⁴R²⁵;
each R^{N} is independently H, C₁-C₆ alkyl or —R²²—NR²⁴R²⁵;
R²⁴ and R²⁵ are each independently selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; —C(O)—R²⁸, where R²⁸ is a Sugar unit of formula (XII) or (XIII); or —NR²⁴R²⁵ together from a C₃-C₈ heterocycle), provided that R²⁴ and R²⁵ are not both H;
R²⁶ is H or C₁-C₆ alkyl;
each n20 is independently 0 to 26, with the proviso that at least one n20 is 2 to 26; and
each n21 is independently 0 to 26, with the proviso that at least one n21 is 2 to 26.

Embodiment 37. The Linker compound of any one of Embodiments 1 to 36, comprising a Polar group having a formula selected from the following, or a stereoisomer or salt thereof:

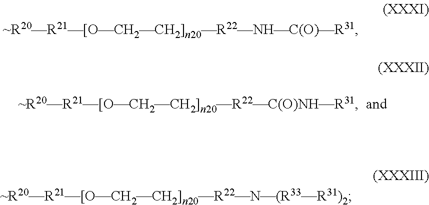

wherein:
R²⁰ is an attachment group to site R^{b}, or to the enzyme-cleavable group;
R²¹ and R²² are each, independently, bond or C₁-C₃ alkylene groups;
R³¹ is a branched polyethylene glycol chain, each branch having 1 to 26 ethylene glycol subunits and each branch having an R³⁵ at its terminus;
R³³ is C₁-C₃ alkylene, —C₁-C₃ alkylene-C(O), —C(O)—C₁-C₃ alkylene or —C(O)—C₁-C₃ alkylene-C(O);
R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; the wavy (˜) line indicates an attachment site to R²⁰; and n20 is 2 to 26.

Embodiment 38. The Linker compound of any one of Embodiments 1-37, comprising a Polar group formed from a precursor group selected from the following:

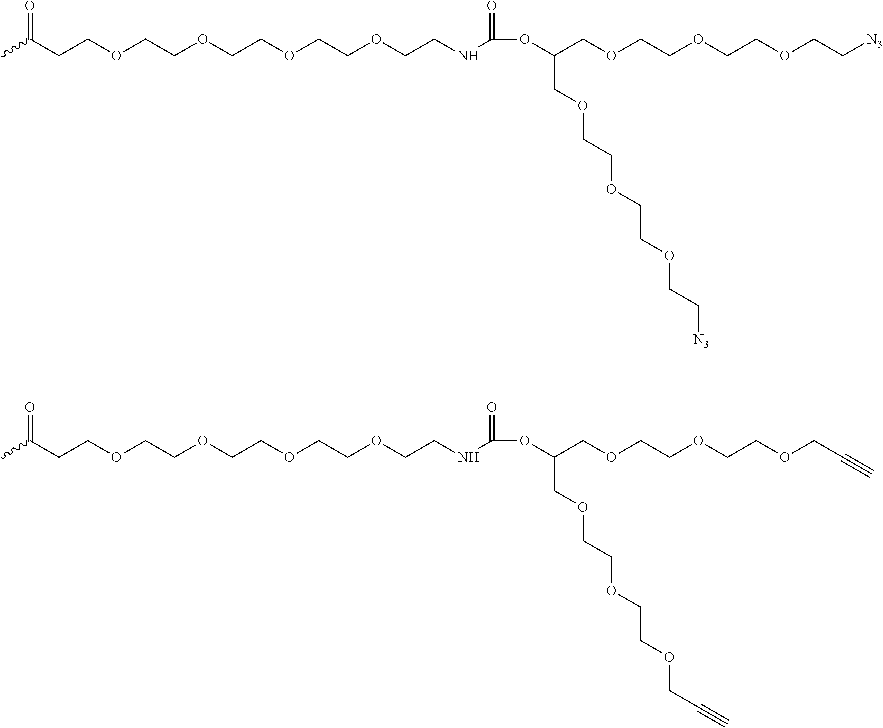

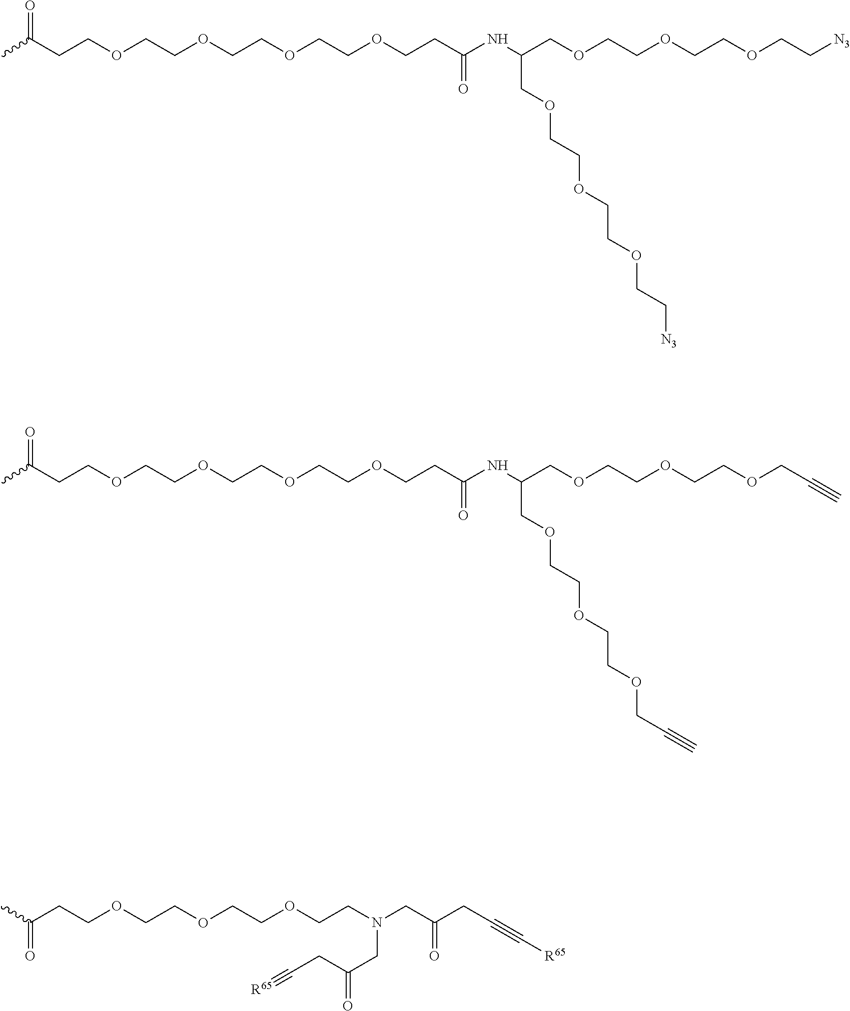

wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and the wavy line is an attachment site to R^{b}, or to the enzyme-cleavable group.

Embodiment 39. The Linker compound of any one of Embodiments 36 to 38, wherein the attachment site to R^{b}, or to the enzyme-cleavable group is formed from a functional group of a precursor compound of the Polar group, said functional group selected from halo, aldehyde, carboxyl, amino, alkynyl, azido, hydroxyl, carbonyl, carbamate, thiol, urea, thiocarbamate, thiourea, sulfonamide, acyl sulfonamide, alkyl sulfonate, triazole, azadibenzocyclooctyne, hydrazine, carbonylalkylheteroaryl, and protected forms thereof.

Embodiment 40. The Linker compound of any one of Embodiments 1-39, comprising a Polar group having a formula:

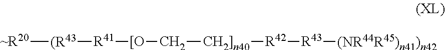

or a stereoisomer or salt thereof, wherein:
R²⁰ is an attachment group to site R^{b}, or to the enzyme-cleavable group;
R⁴¹ and R⁴² are each, independently, bond or C₁-C₆ alkylene;
each R⁴³ is, independently, a bond or is selected from C₁-C₁₂ alkylene, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)—, —C(O)—C₁-C₁₂ alkylene-NH—, —NH—C(O)—NH—, —NH—C(O)—, —NH—C(O)—C₁-C₁₂ alkylene, —C(O)—NH—C₁-C₁₂ alkylene, -heteroarylene, heteroaryl-C₁-C₁₂ alkylene, heteroaryl-C₁-C₁₂ alkylene-C(O)—, or —C(O)NR⁴⁶R⁴⁷, wherein one of R⁴⁶ and R⁴⁷ is H or C₁-C₁₂ alkylene and the other is C₁-C₁₂ alkylene;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate; n40 is 2 to 26;
n41 is 1 to 6; and
n42 is 1 to 6.

Embodiment 41. The Linker compound of any one of Embodiments 1-40, comprising a Polar group having a formula:

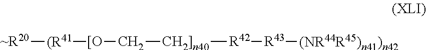

or a stereoisomer or salt thereof, wherein:
R²⁰ is an attachment group to site R^{b}, or to the enzyme-cleavable group;
R⁴¹ and R⁴² are each, independently, bond or C₁-C₆ alkylene;
R⁴³ is a bond or is selected from C₁-C₁₂ alkylene, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)—, —C(O)—C₁-C₁₂ alkylene-NH—, —NH—C(O)—NH—, —NH—C(O)—, —NH—C(O)—C₁-C₁₂ alkylene, C(O)—NH—C₁-C₁₂ alkylene, -heteroarylene, heteroaryl-C₁-C₁₂ alkylene, heteroaryl-C₁-C₁₂ alkylene-C(O)—, or —C(O)NR⁴⁶R⁴⁷, wherein one of R⁴⁶ and R⁴⁷ is H or C₁-C₁₂ alkylene and the other is C₁-C₁₂ alkylene;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate; n40 is 1 to 26;
n41 is 1 to 6; and
n42 is 1 to 6.

Embodiment 42. The Linker compound of any one of Embodiments 1-41, comprising a Polar group having a formula:

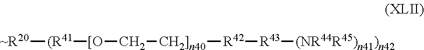

or a stereoisomer or salt thereof, wherein:
R²⁰ is an attachment group to site R^{b}, or to the enzyme-cleavable group;
R⁴¹ and R⁴² are each, independently, bond or C₁-C₃ alkylene;
R⁴³ is a bond or is selected from C₁-C₆ alkylene, —NH—C₁-C₁₂ alkylene, —C₁-C₆ alkylene-NH—, —C(O)—C₁-C₆ alkylene, —C₁-C₆ alkylene-C(O)—, —NH—C₁-C₆ alkylene-C(O)—, —C(O)—C₁-C₆ alkylene-NH—, —NH—C(O)—NH—, —NH—C(O)—, —NH—C(O)—C₁-C₆ alkylene, —C(O)—NH—C₁-C₁₂ alkylene, -heteroarylene, heteroaryl-C₁-C₆ alkylene, heteroaryl-C₁-C₆ alkylene-C(O)—, or —C(O)NR⁴⁶R⁴⁷, wherein one of R⁴⁶ and R⁴⁷ is H or C₁-C₆ alkylene and the other is C₁-C₁₂ alkylene;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate; n40 is 1 to 16;
n41 is 1 to 4; and
n42 is 1 to 4.

Embodiment 43. The Linker compound of any one of Embodiments 7, 36, 37 and 40-42, wherein R²⁰ is formed from a functional group of a precursor compound of the Polar group, said functional group selected from halo, aldehyde, carboxyl, amino, alkynyl, azido, hydroxyl, carbonyl, carbamate, thiol, urea, thiocarbamate, thiourea, sulfonamide, acyl sulfonamide, alkyl sulfonate, triazole, azadibenzocyclooctyne, hydrazine, carbonylalkylheteroaryl, or protected forms thereof.

Embodiment 44. The Linker compound of any one of Embodiments 7, 36, 37 and 40-42, wherein R²⁰ comprises one of the following structures:

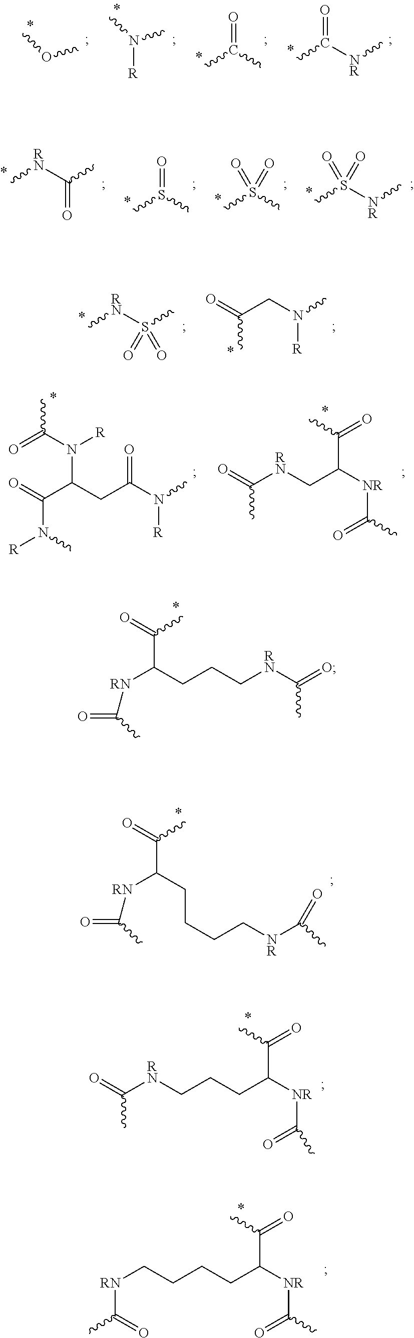

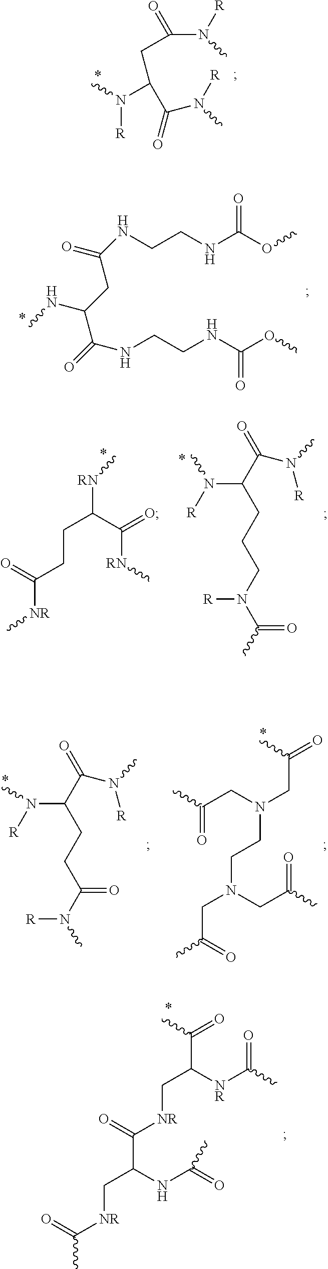

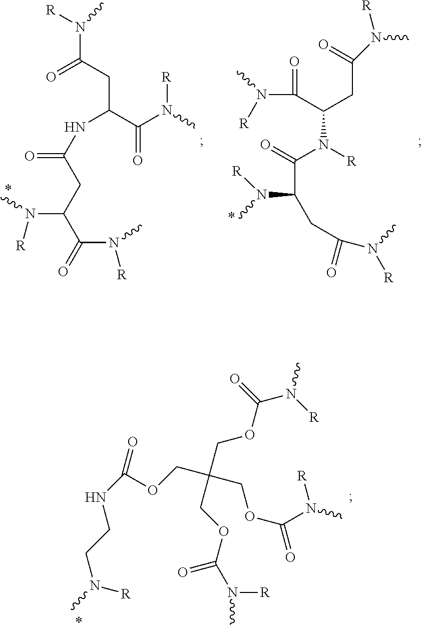

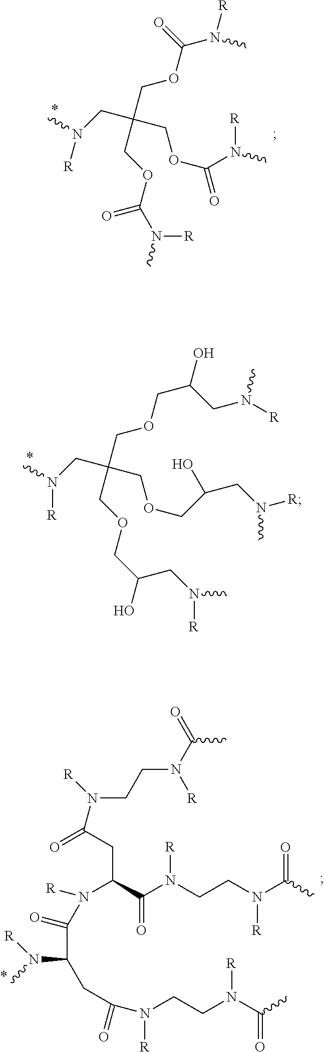

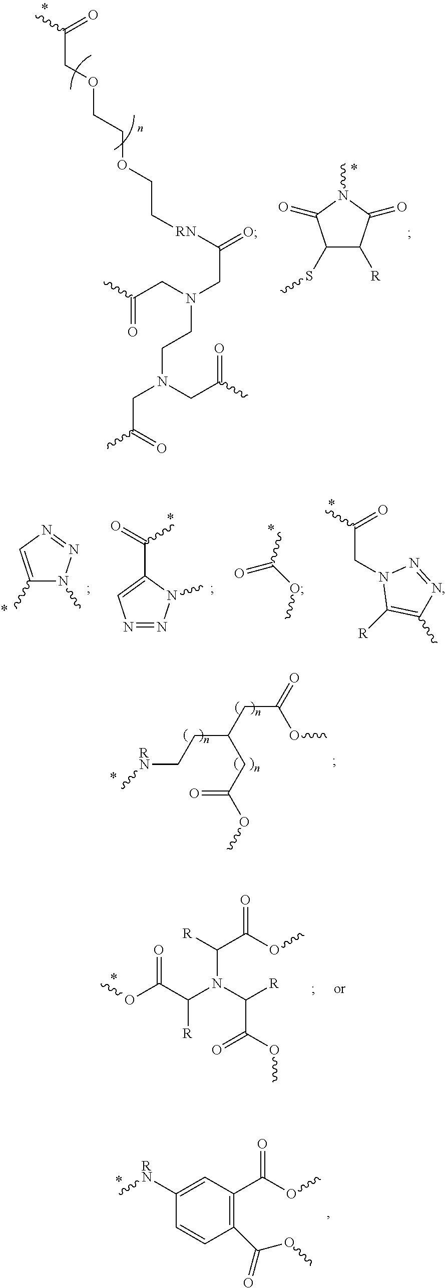

or a stereoisomer thereof, wherein R is H, C₁-C₆ alkyl or polyhydroxyl group, n is 0 to 12, the ( [symbol] *) indicates an attachment site to R^{b}, or to the enzyme-cleavable group, and the ( [symbol] ) indicates an attachment site to a remainder portion of the Polar group.

Embodiment 45. The compound of any one of Embodiments 7, 36, 37 and 40-42, wherein R²⁰ has one of the following structures:

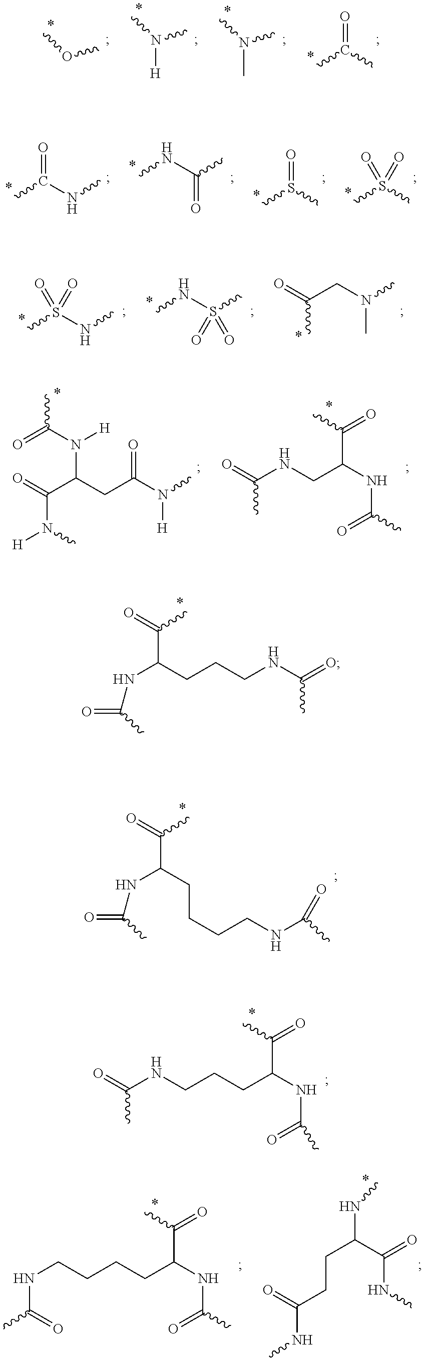

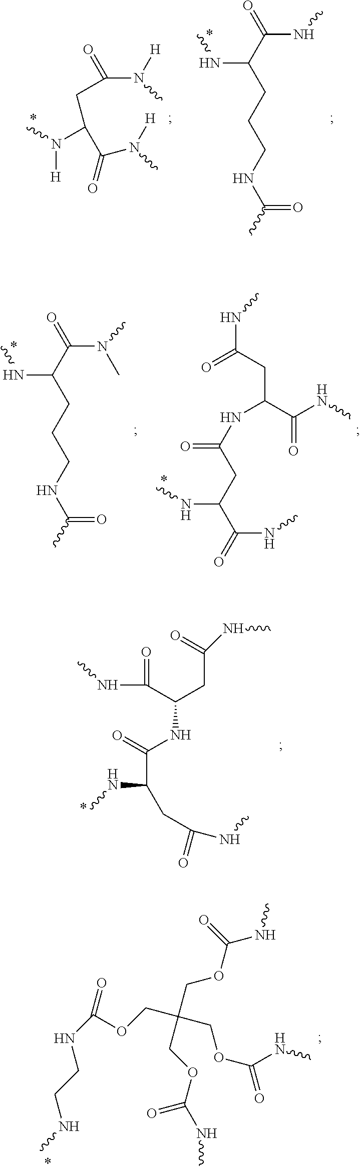

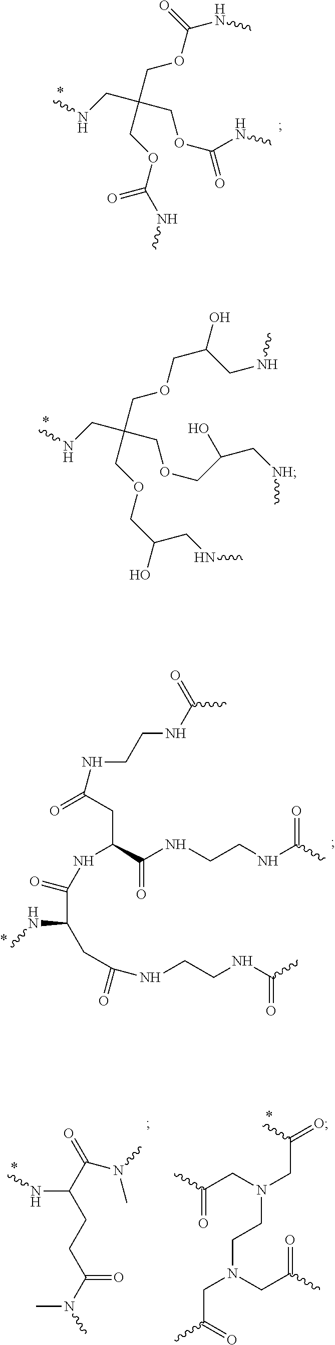

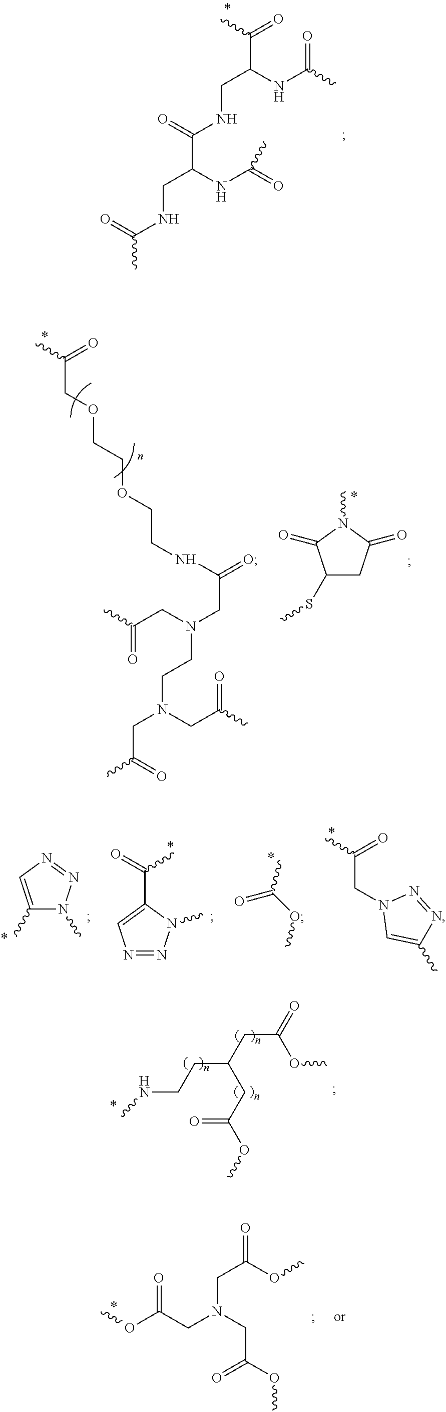

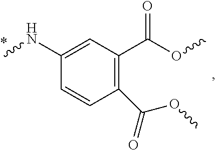

or a stereoisomer thereof, wherein n=0 to 12, the ( [symbol] *) indicates an attachment site to R^{b}, or to the enzyme-cleavable group, and the ( [symbol] ) indicates an attachment site to a remainder portion of the Polar group.

Embodiment 46. The Linker compound of any one of Embodiments 40-45, wherein R⁴³—(NR⁴⁴R⁴⁵)n₄₁ has one of the following structures:

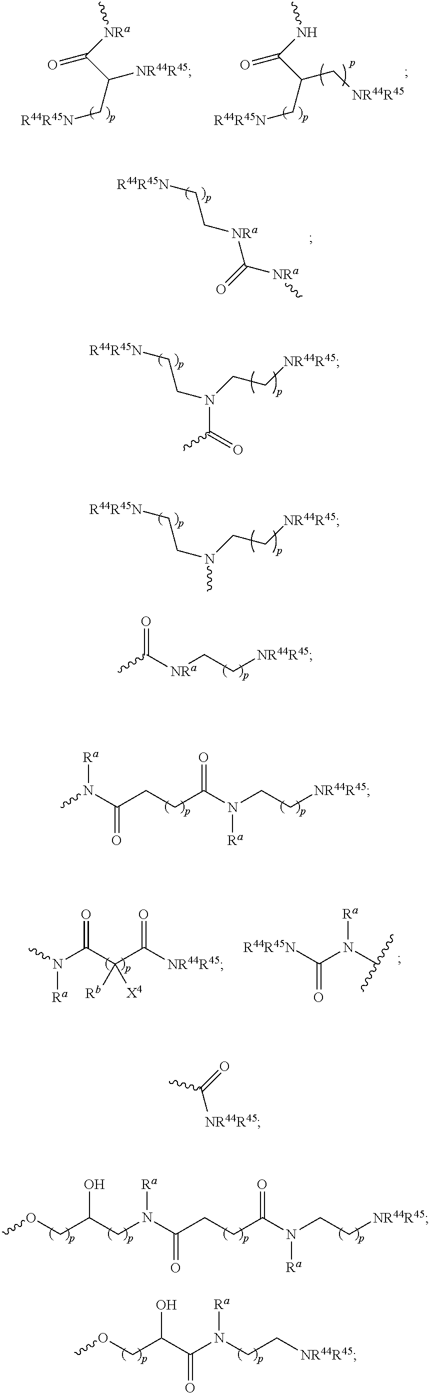

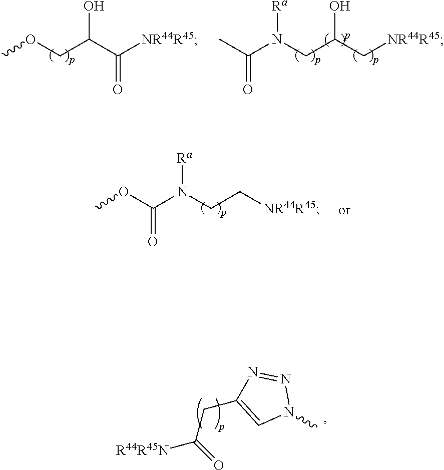

or a stereoisomer thereof, wherein R=H, C₁-C₆ alkyl, a polyhydroxyl group, or a substituted polyhydroxyl group; and the ( [symbol] ) indicates the attachment site of R⁴³ to the remainder of the Polar group.

Embodiment 47. The Linker compound of any one of Embodiments 40-45, wherein R⁴³—(NR⁴⁴R⁴⁵)n₄₁ has one of the following structures:

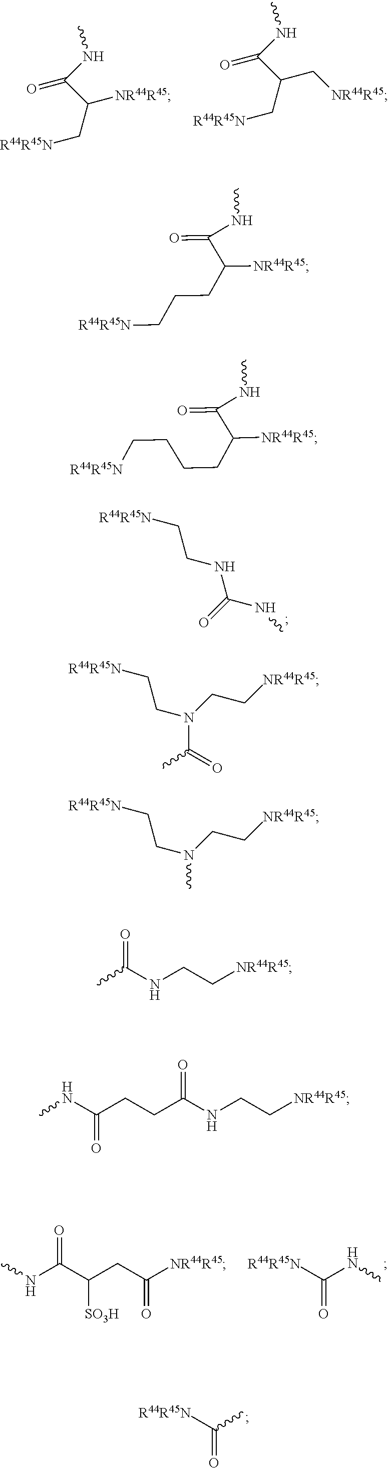

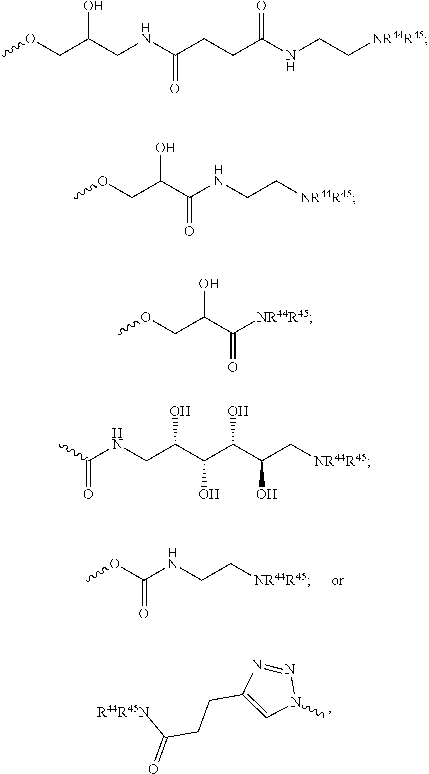

or a stereoisomer thereof, wherein the ( [symbol] ) indicates the attachment site of R⁴³ to the remainder of the Polar group.

Embodiment 48. The Linker compound of any one of Embodiments 40-47, wherein —NR⁴⁴R⁴⁵ has one of the following structures:

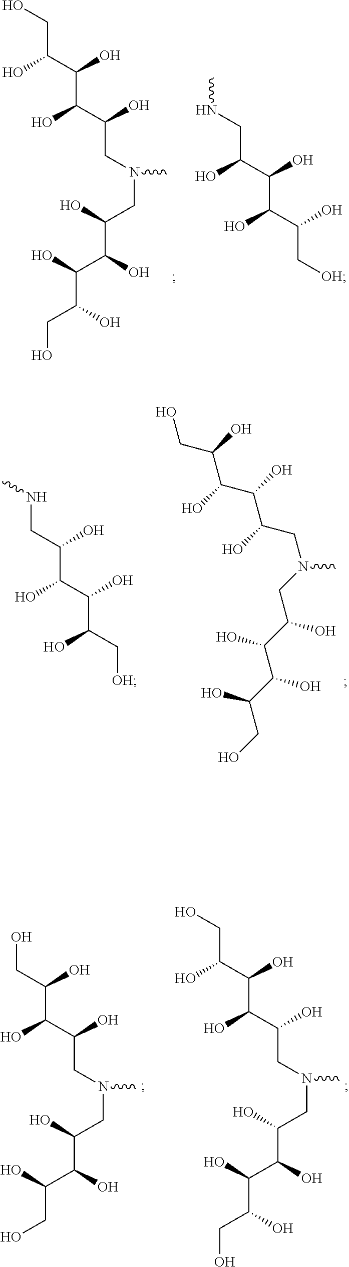

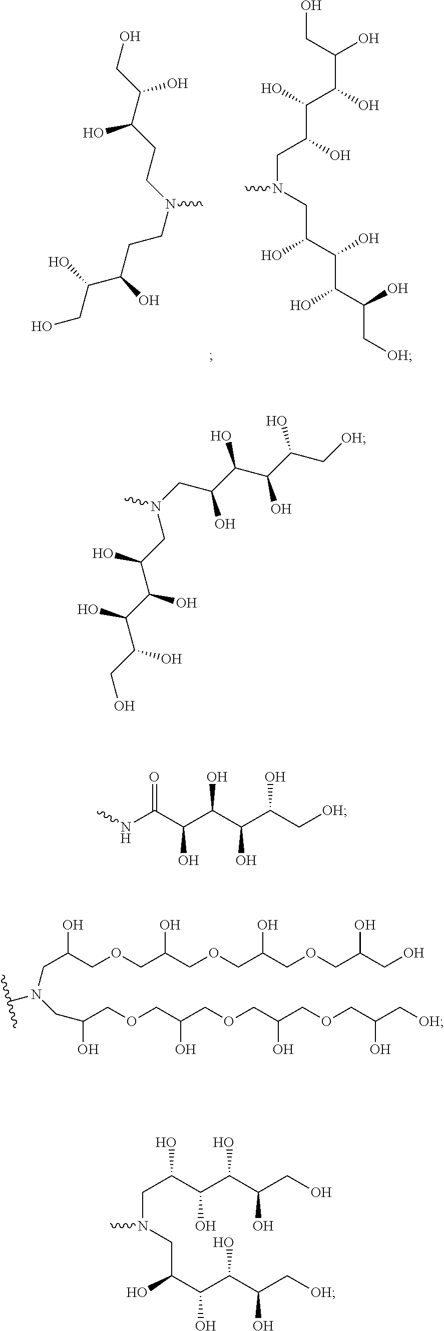

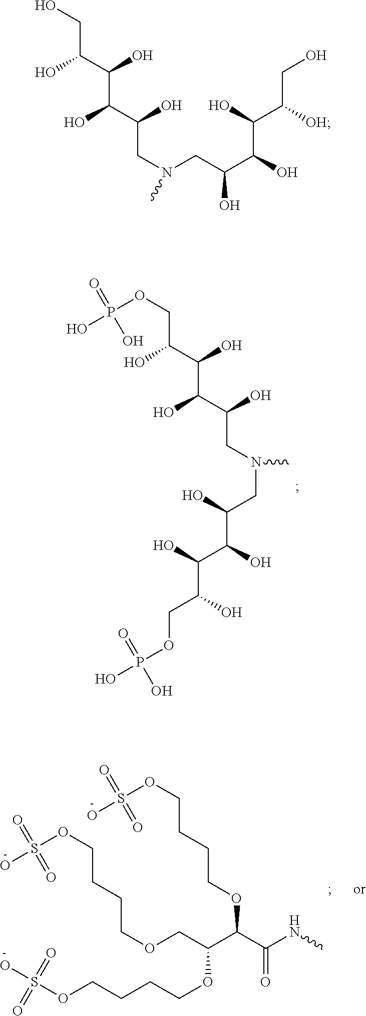

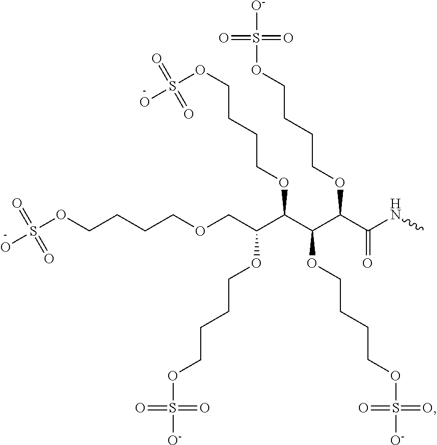

or a stereoisomer thereof, wherein the ( [symbol] ) indicates the attachment site of —NR⁴⁴R⁴⁵ to the remainder of the Polar group.

Embodiment 49. The Linker compound of any one of Embodiments 1-48, comprising a Polar group having one of the following structures prior to attachment to the Linker Unit:

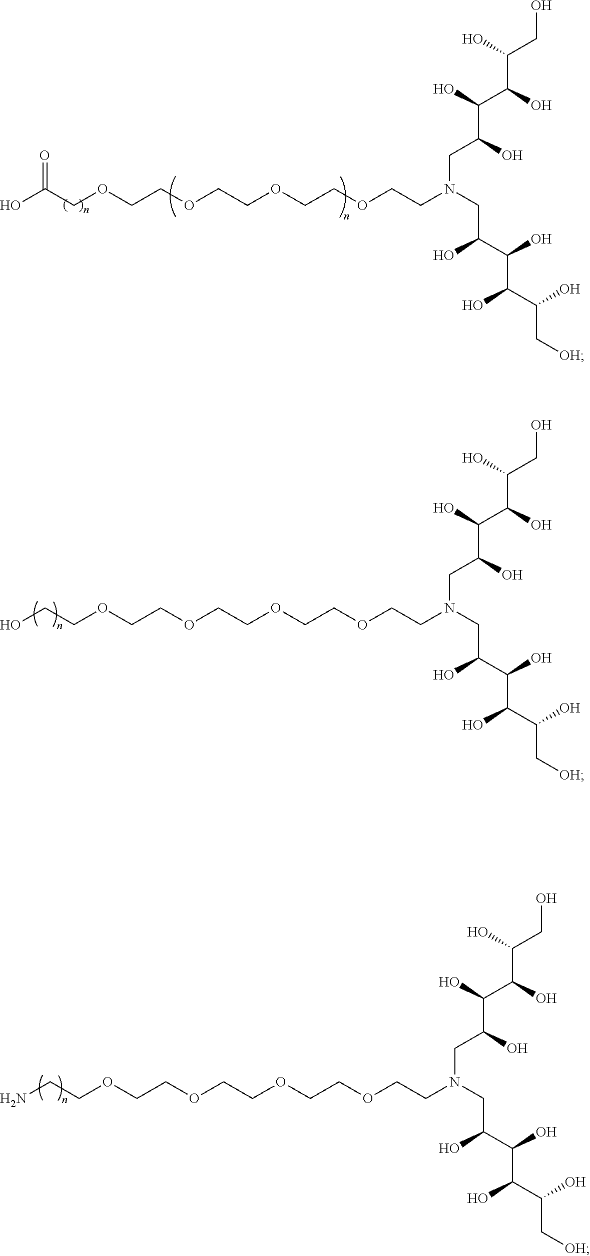

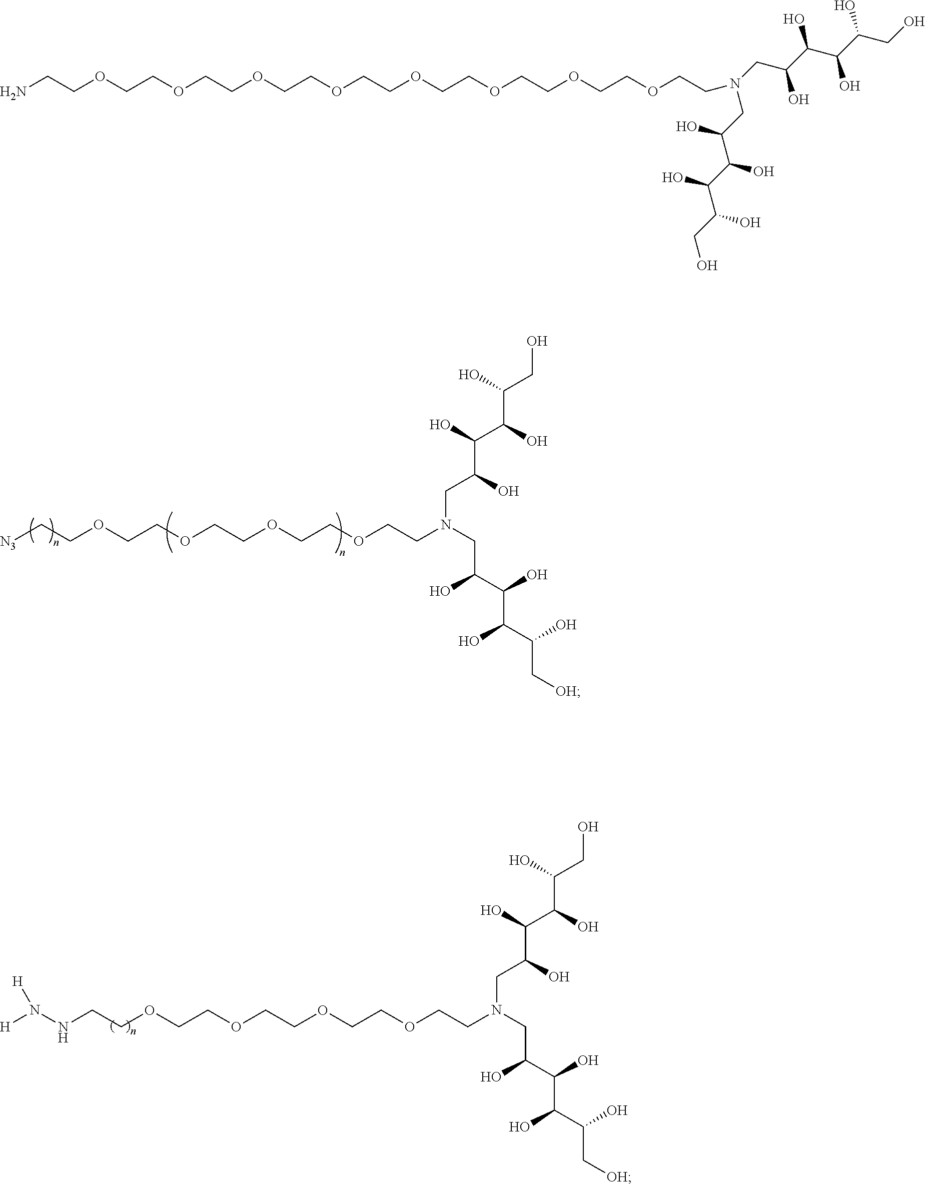

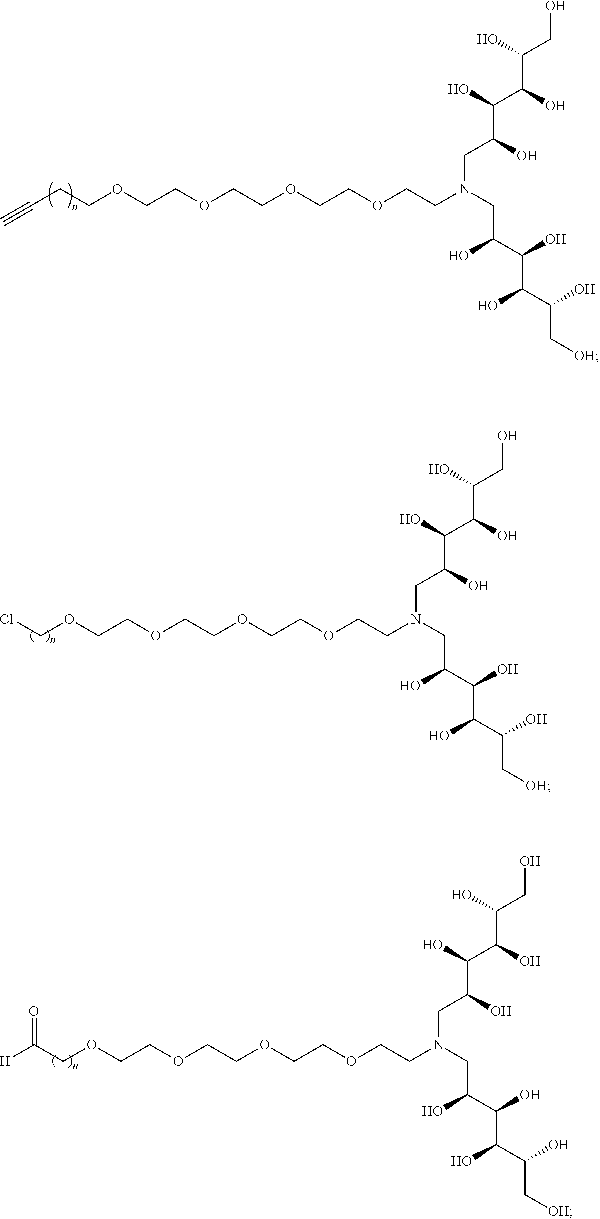

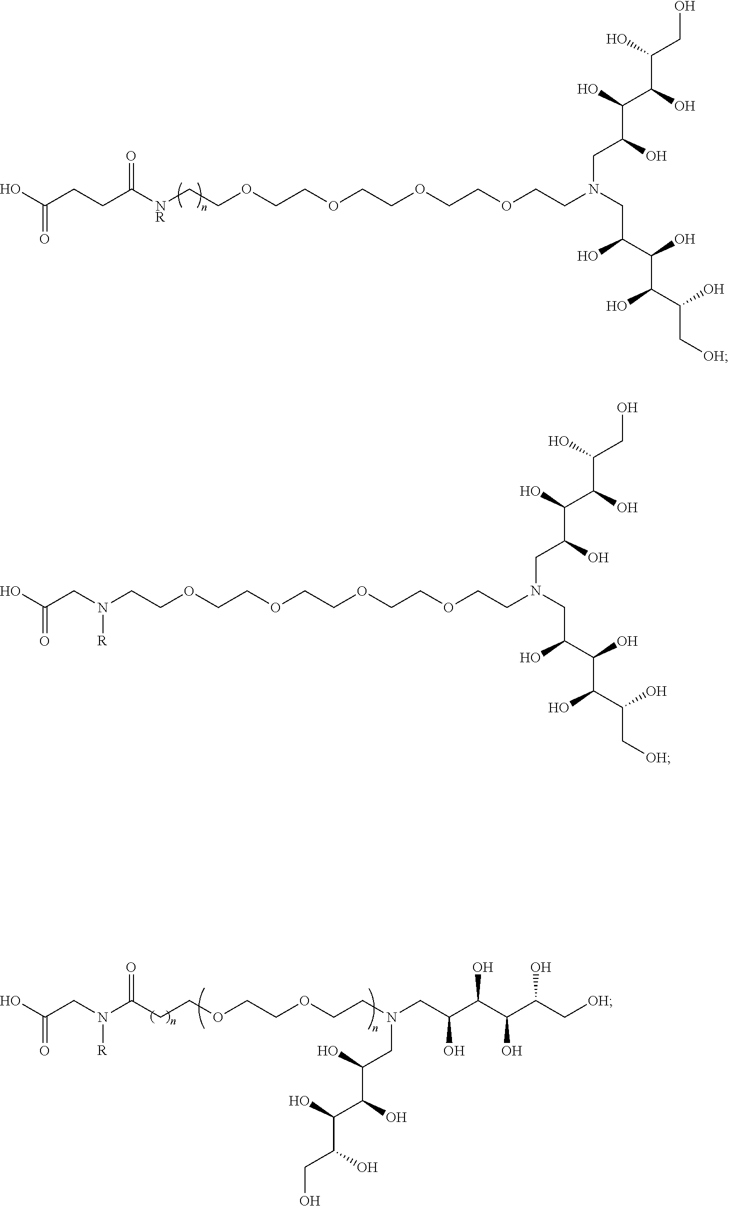

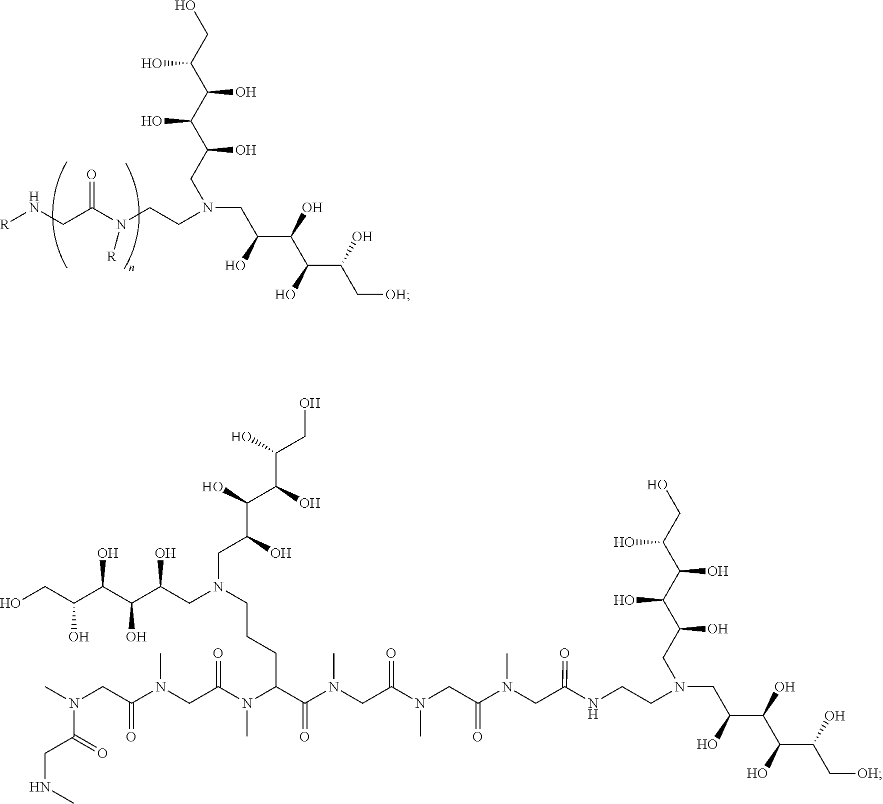

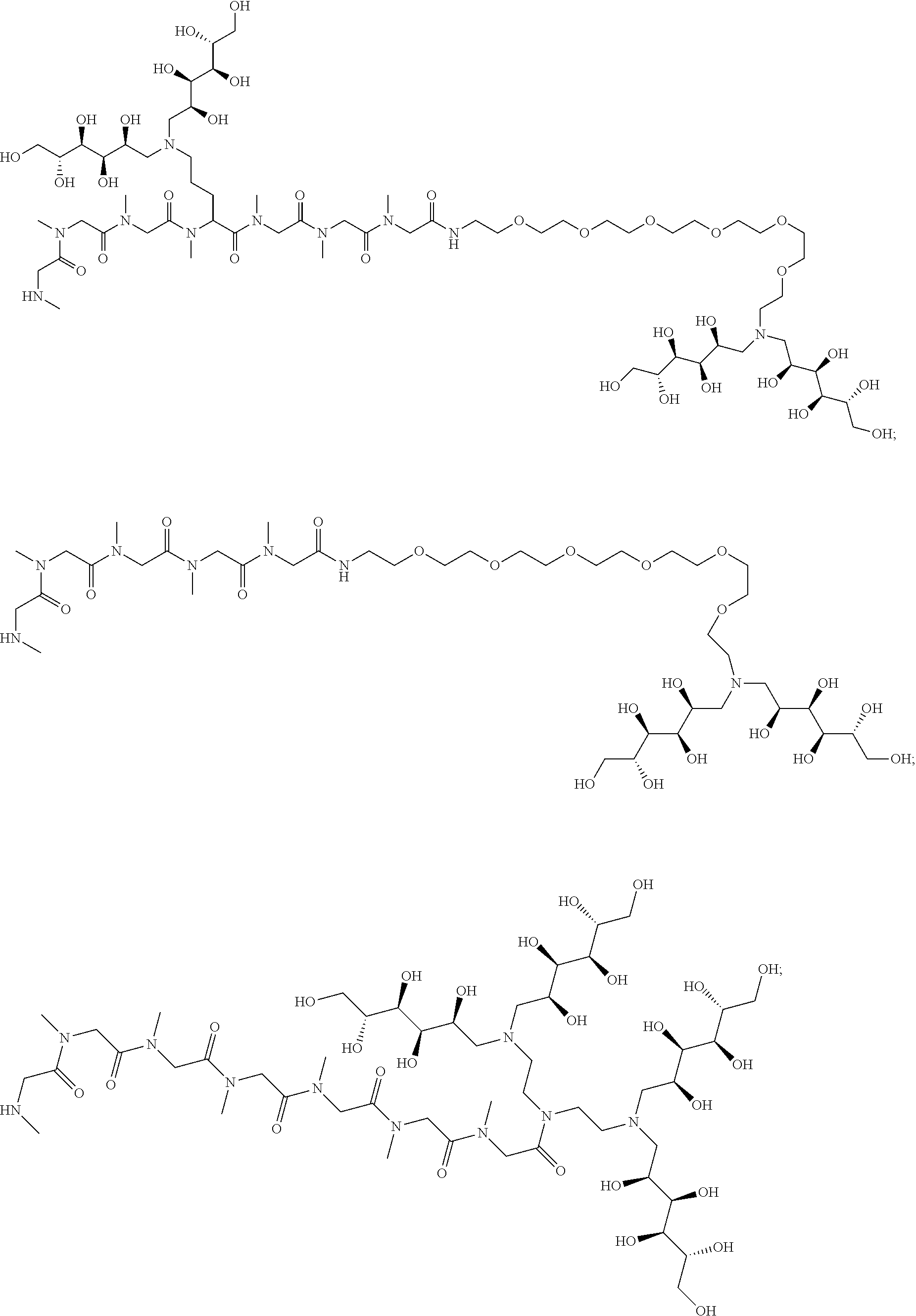

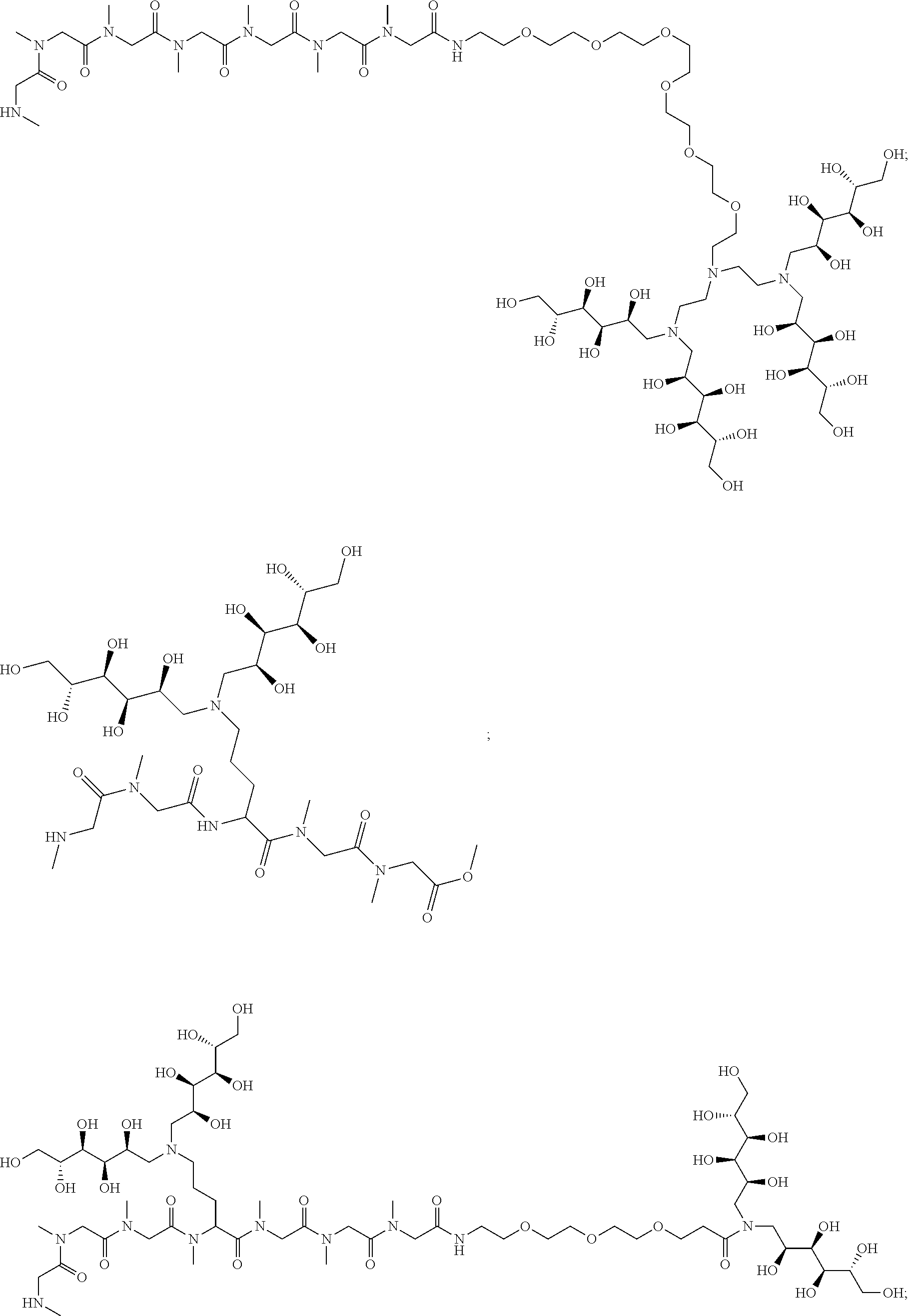

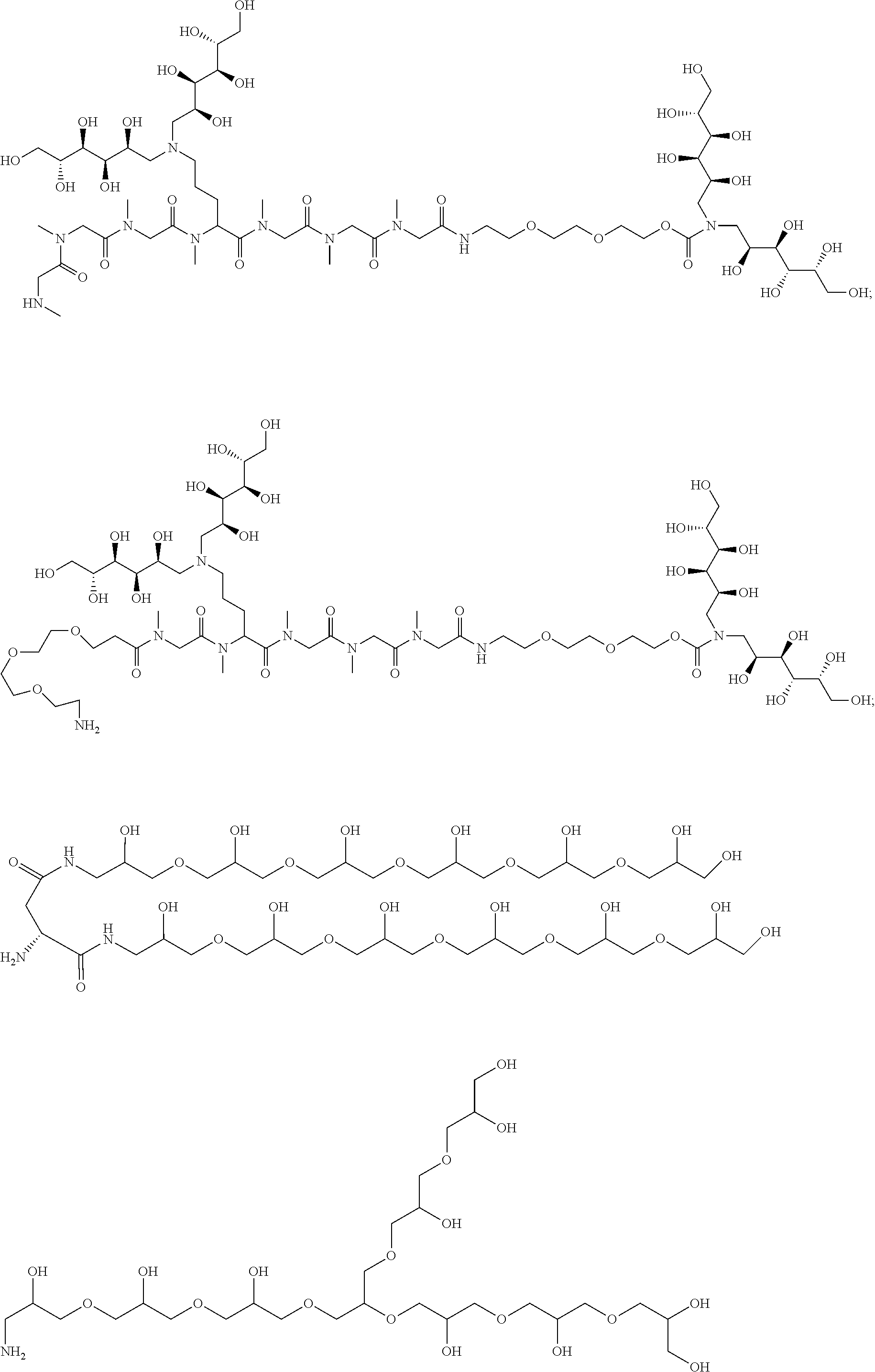

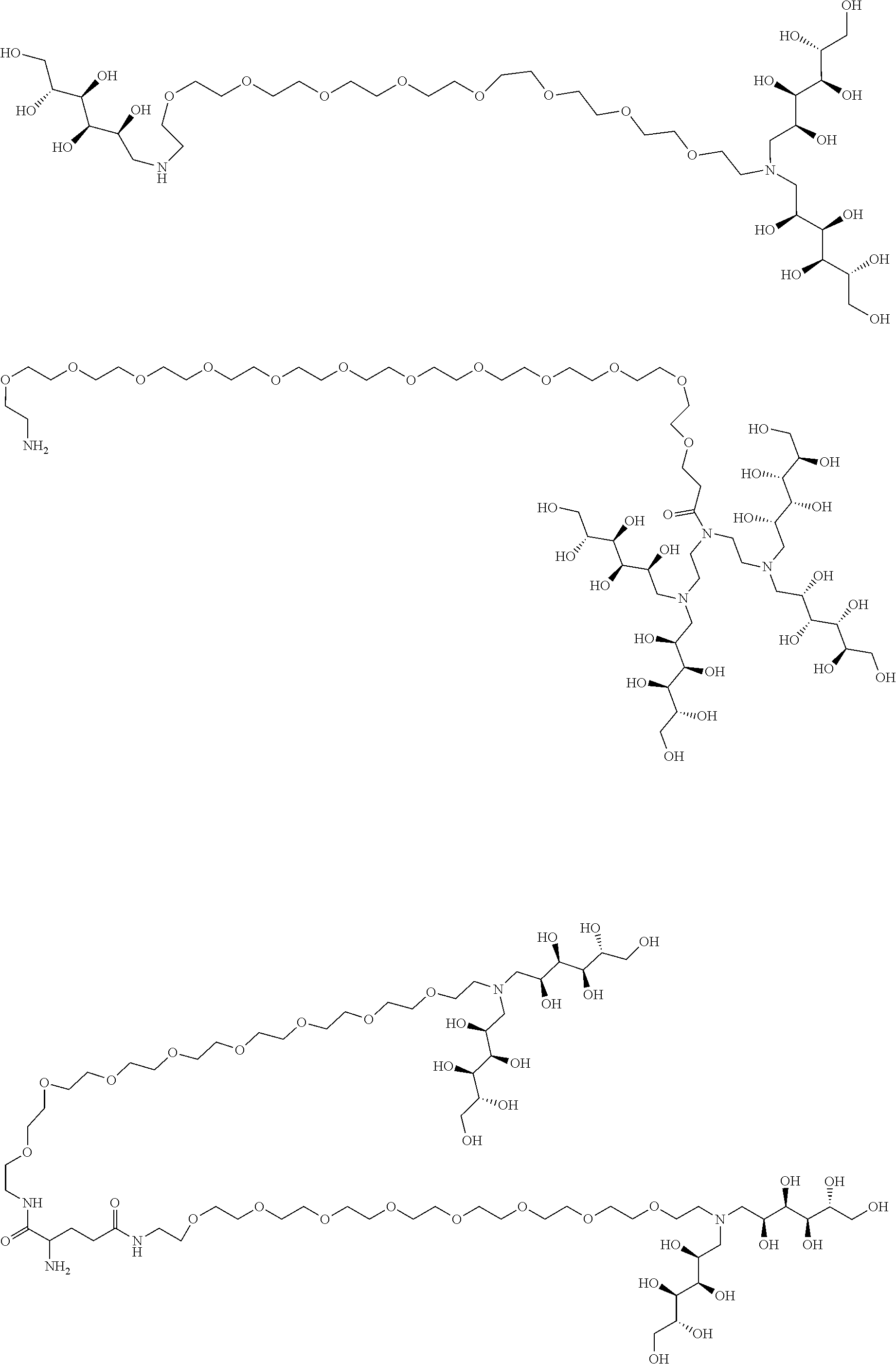

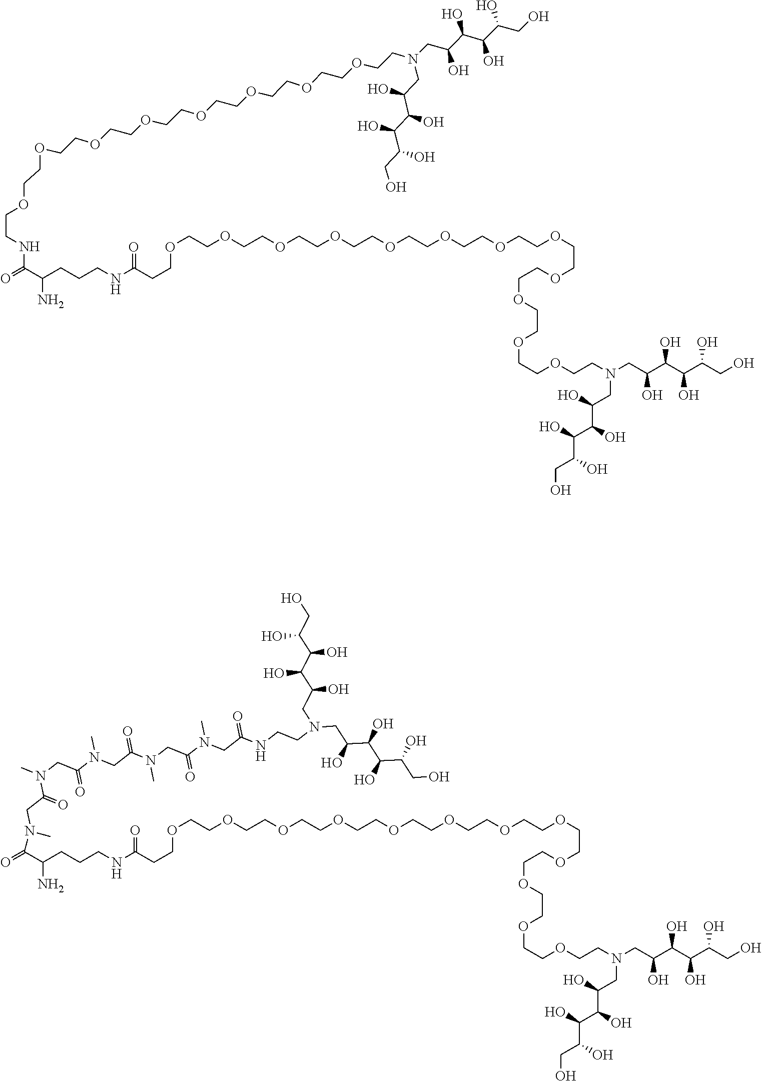

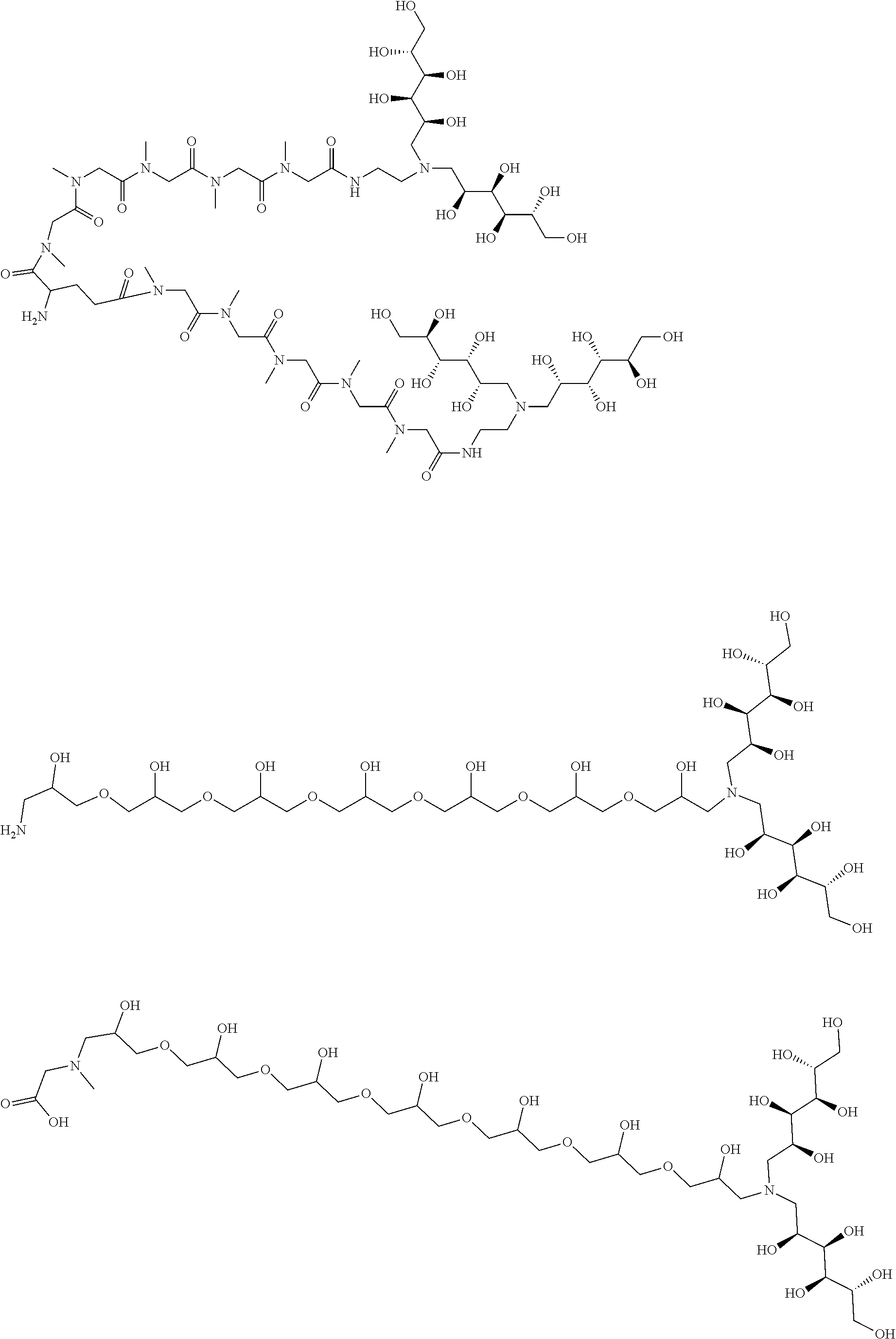

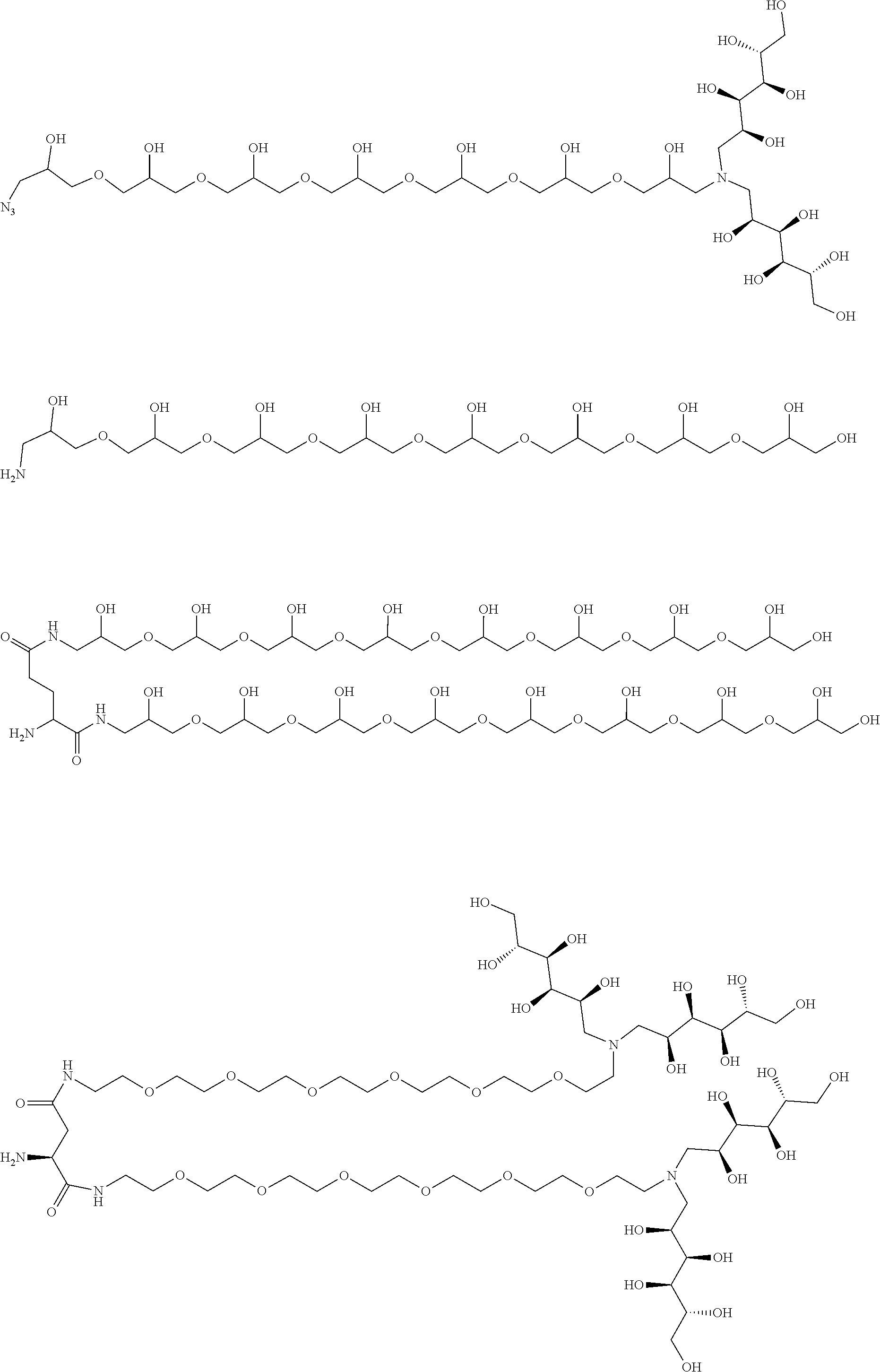

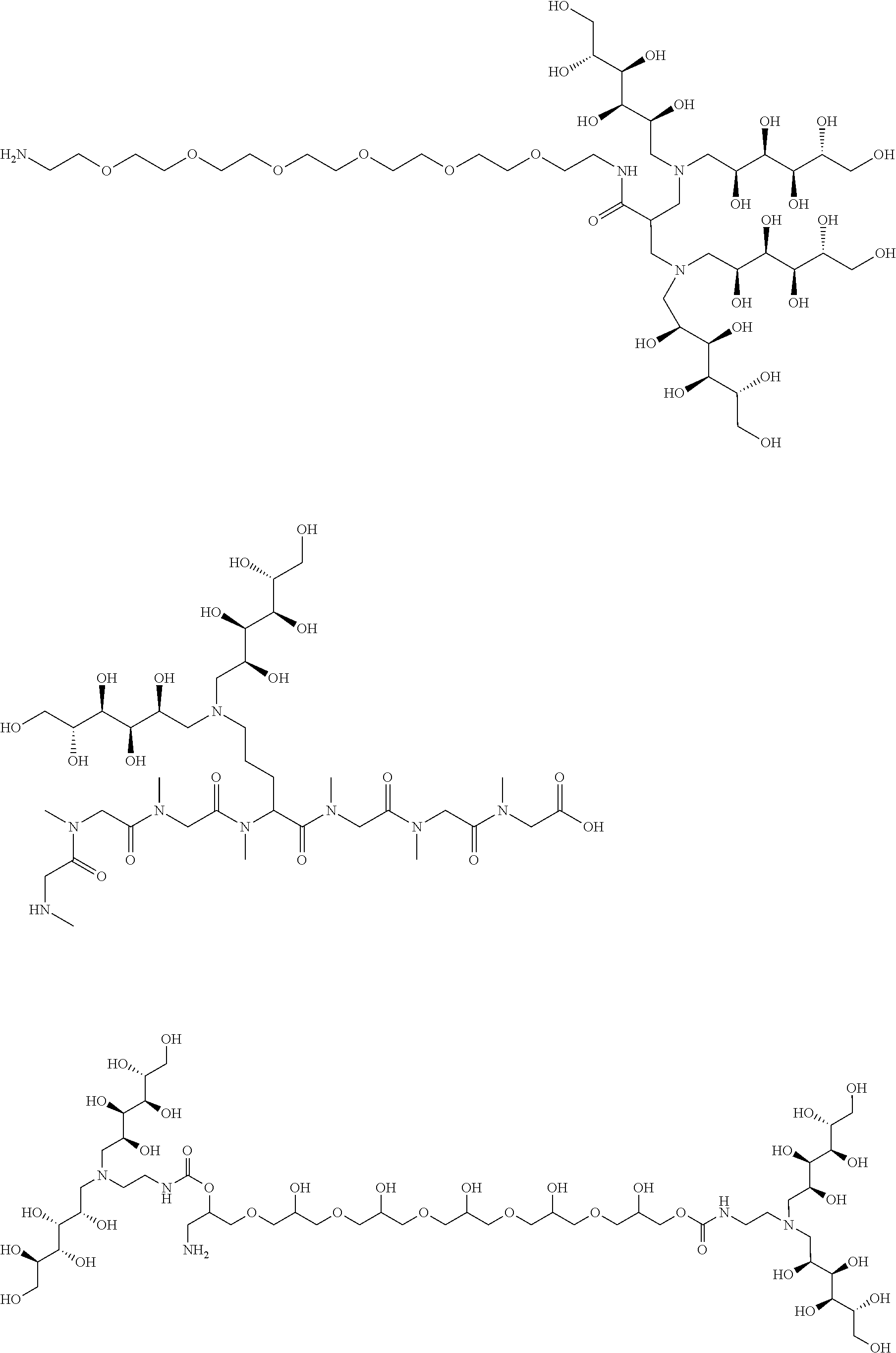

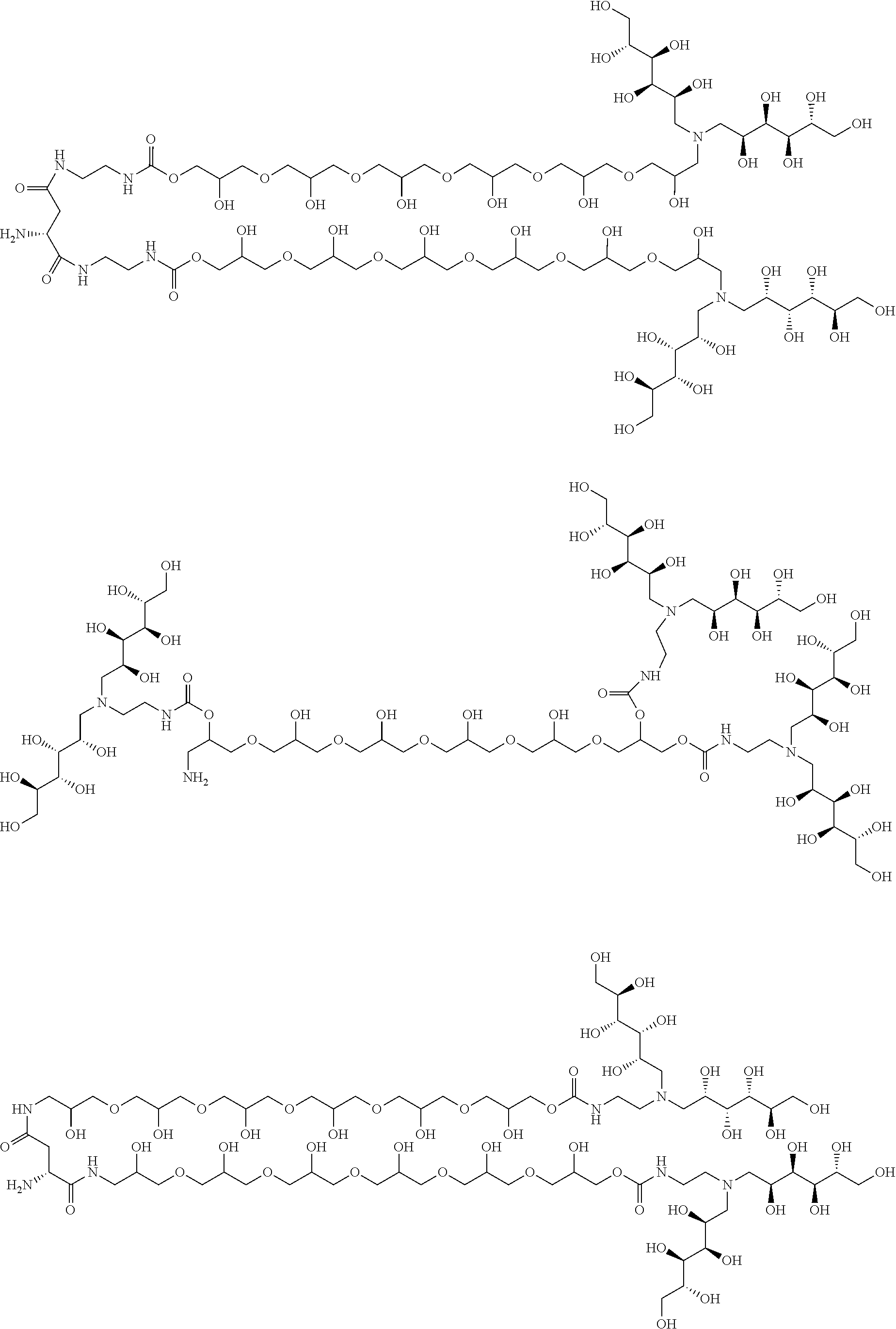

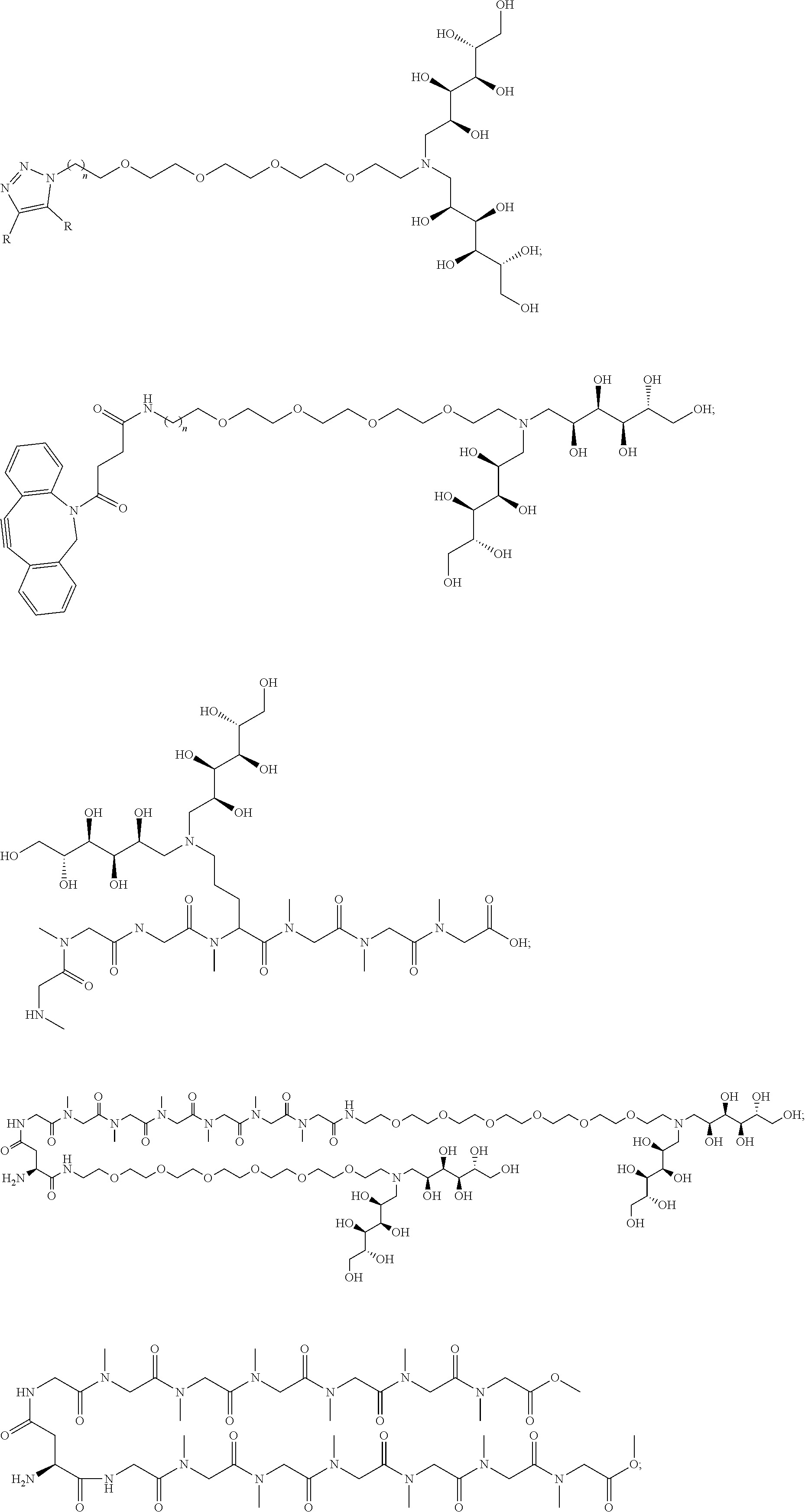

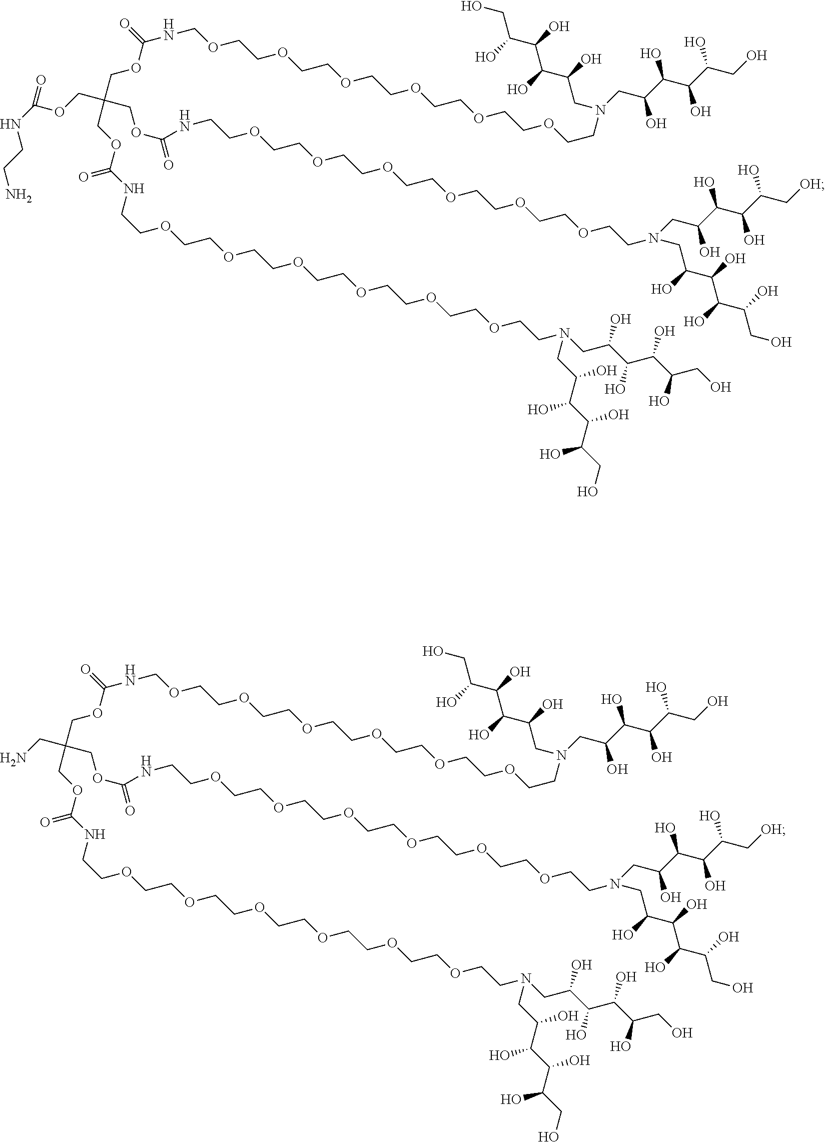

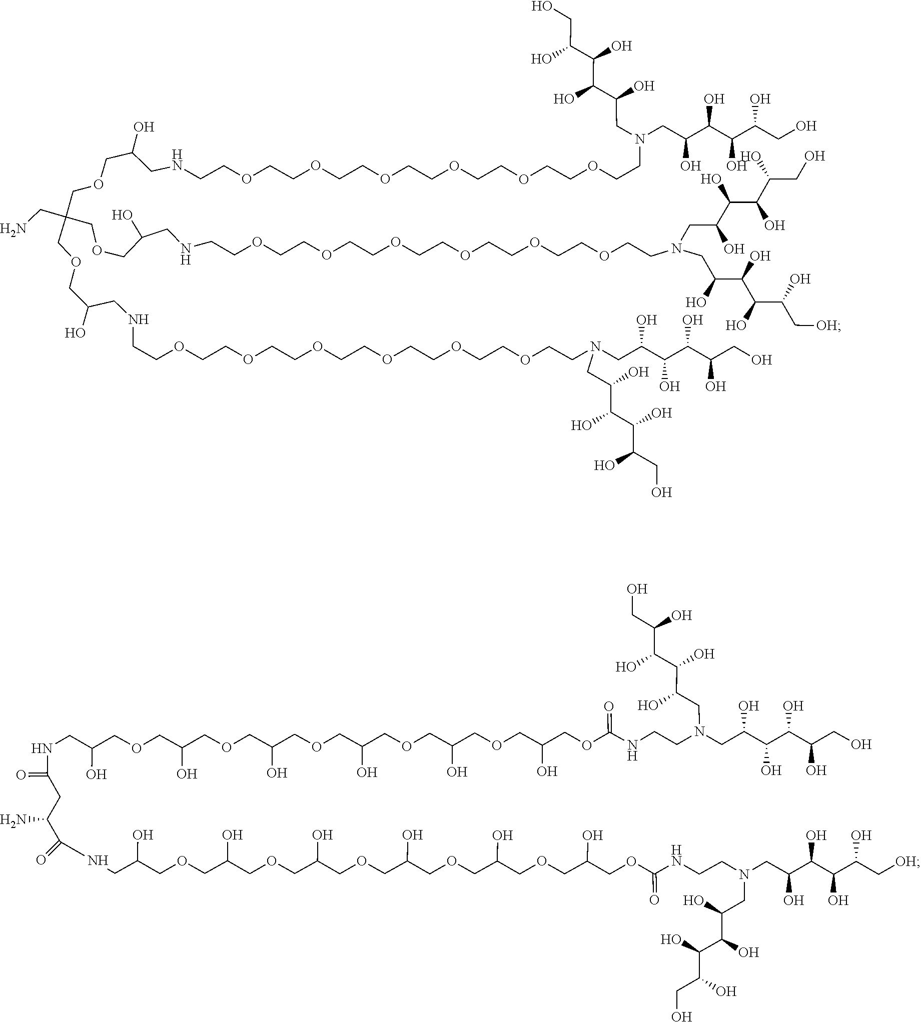

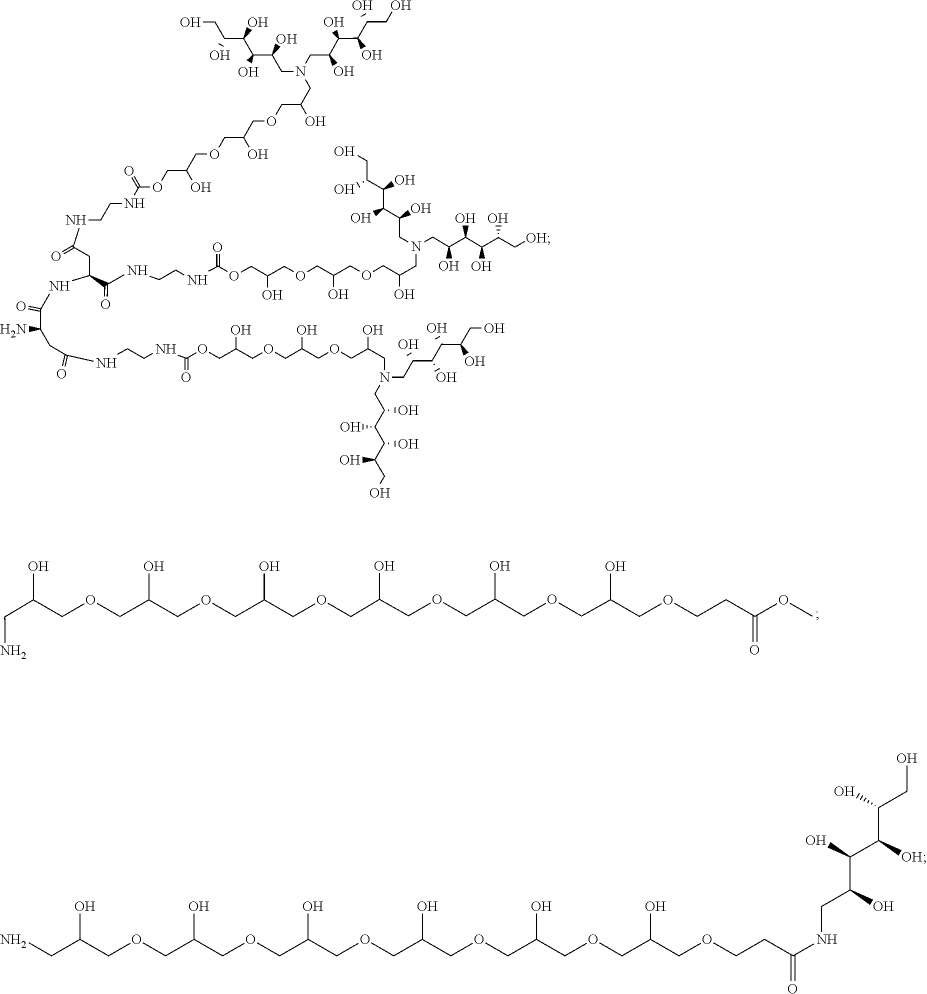

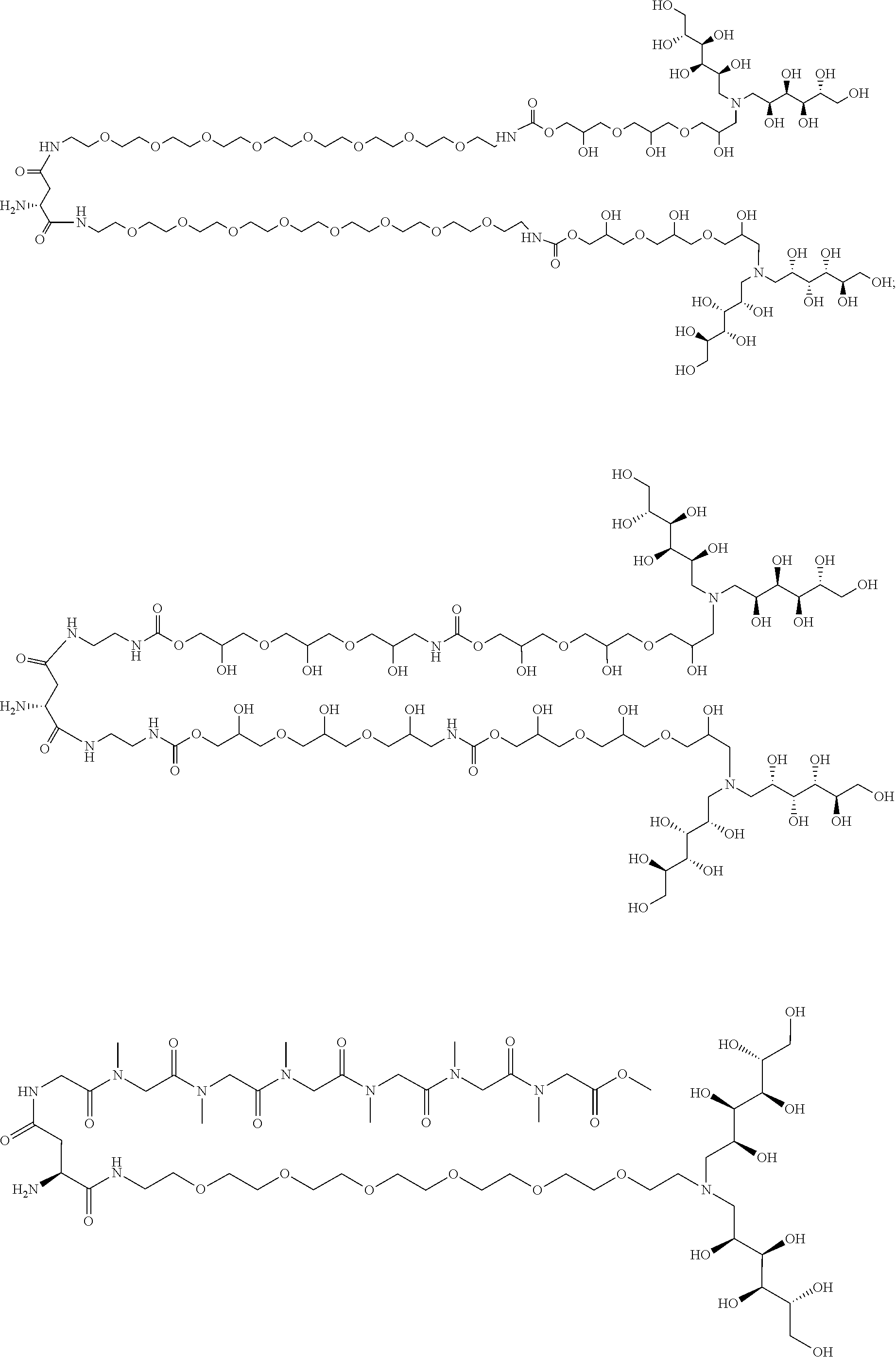

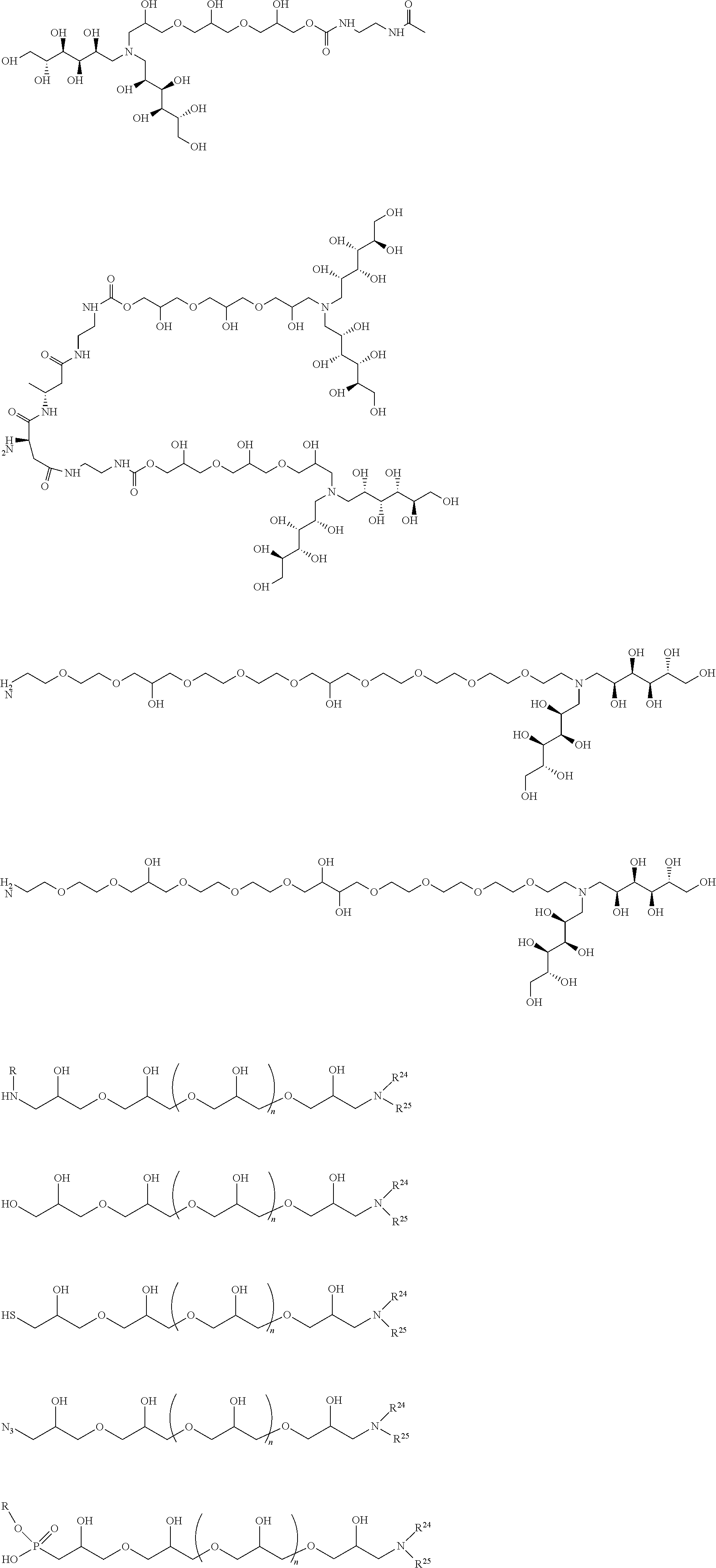

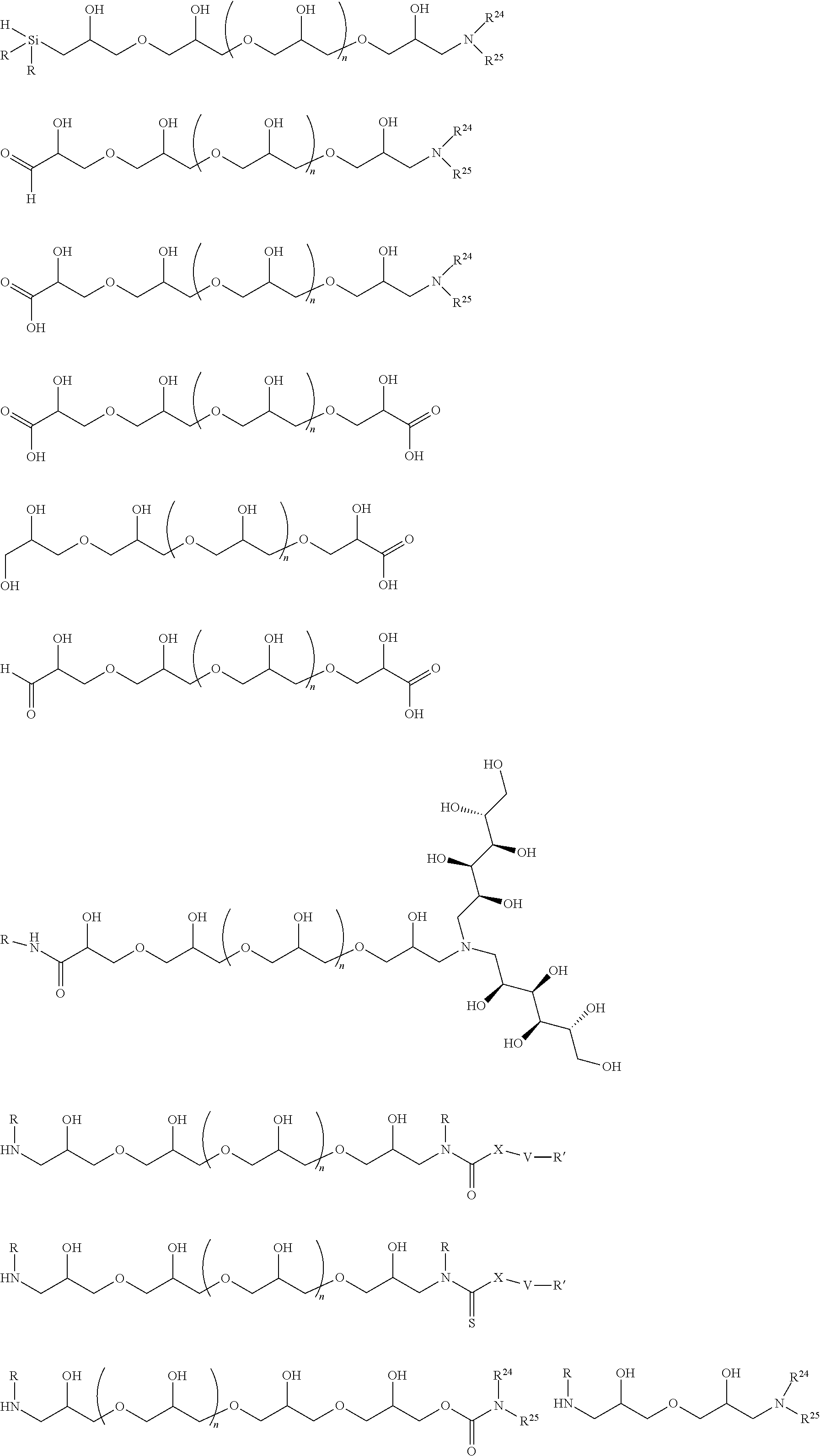

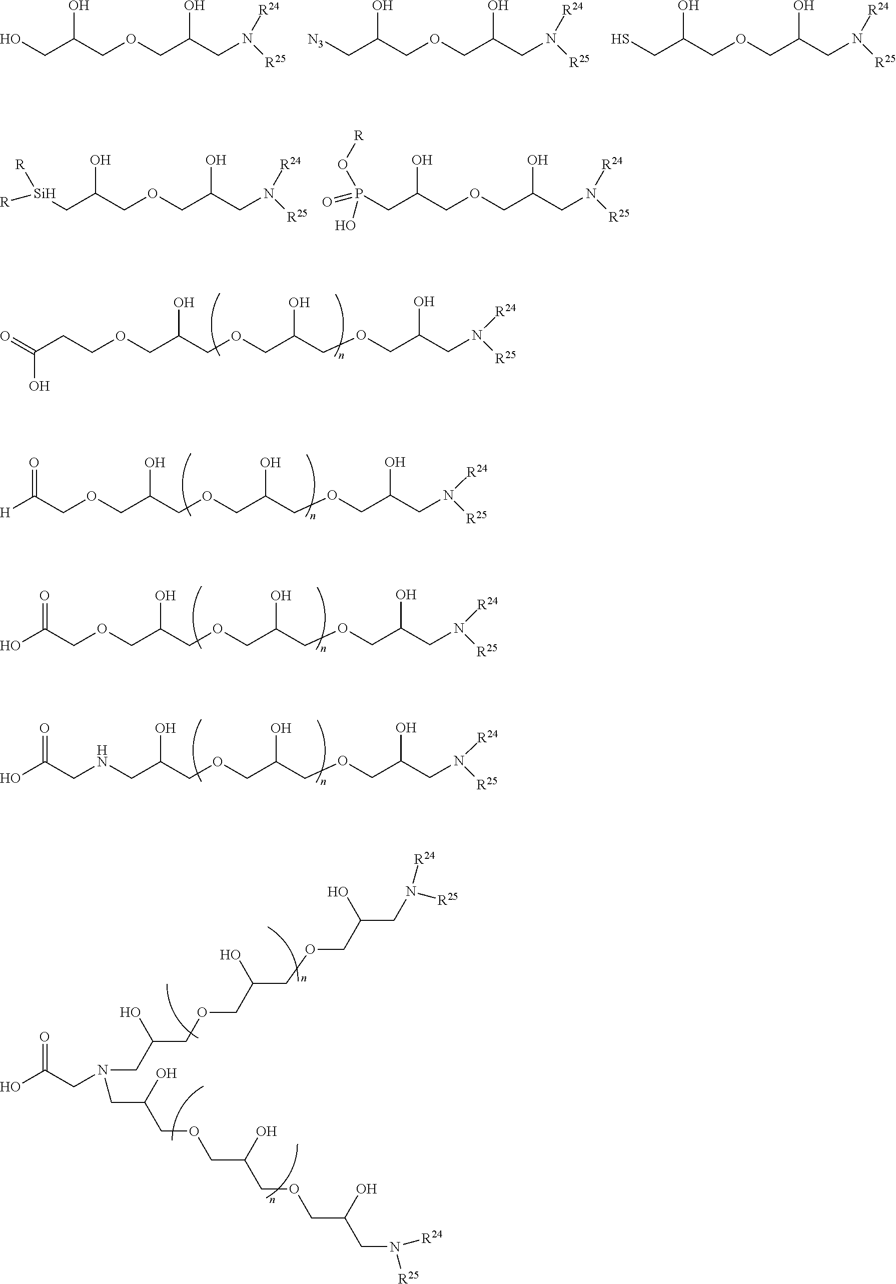

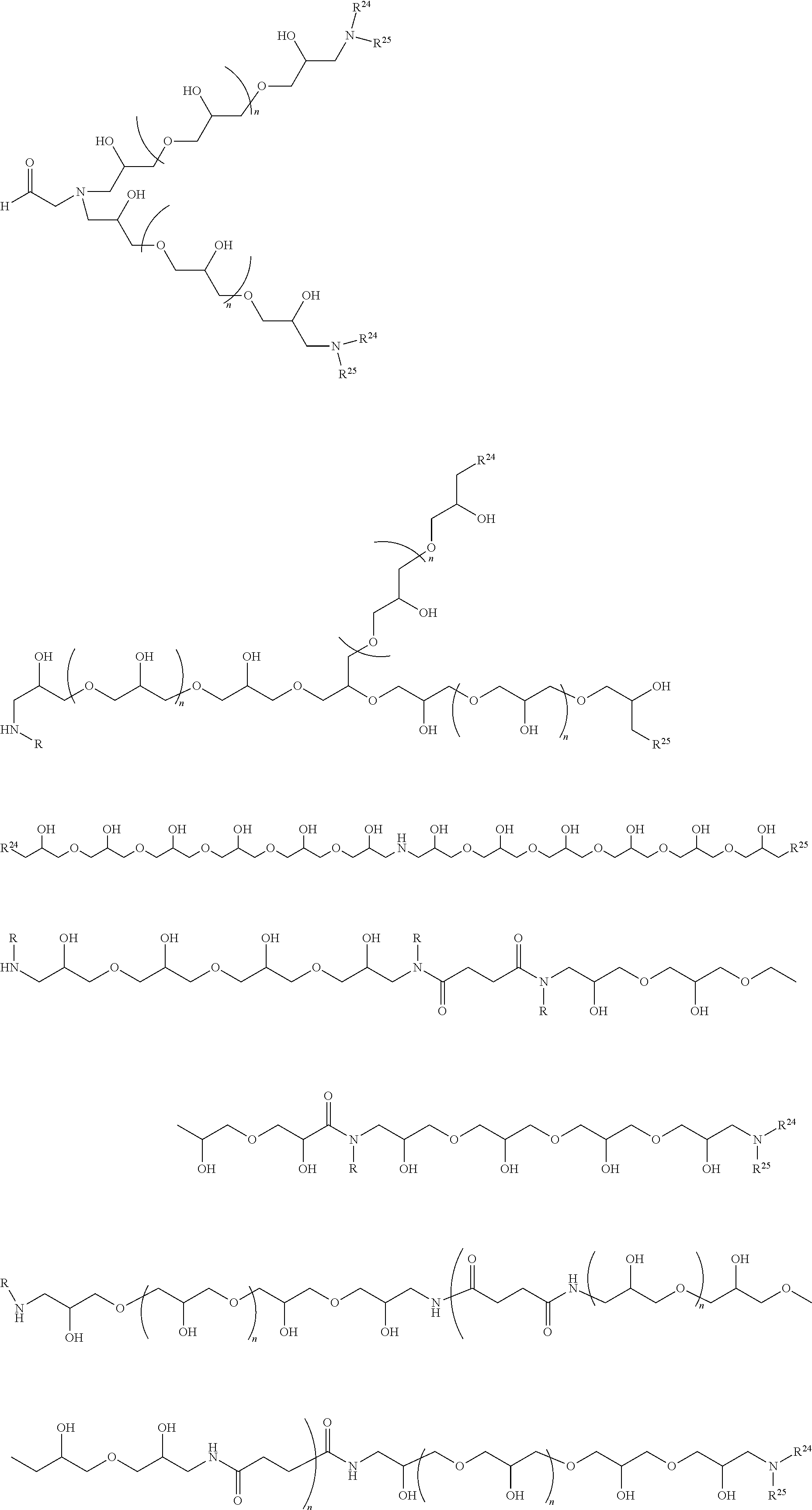

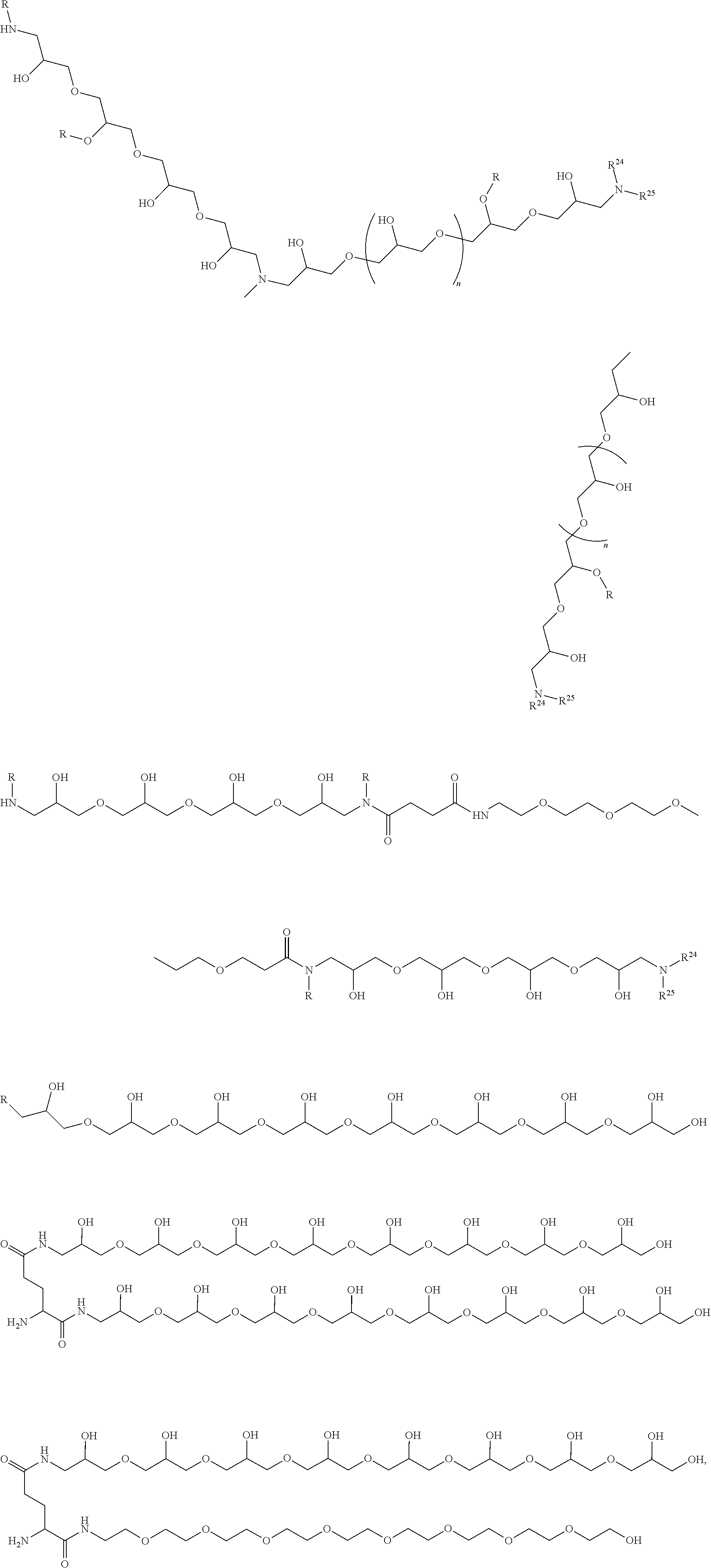

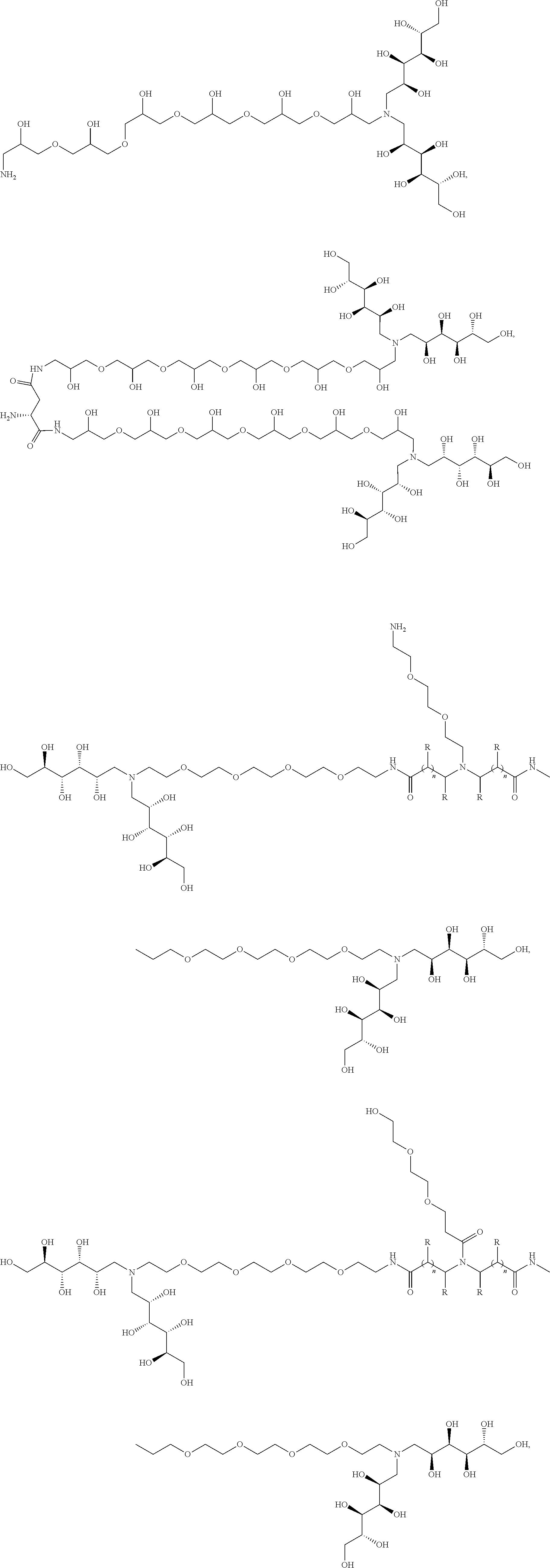

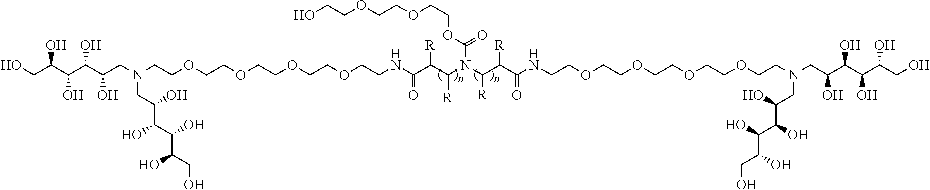

wherein:
each R is independently H or C₁-C₆ alkyl;
R′ is H, C₁-C₆ alkyl, —N(R²⁴)(R²⁵) or —CO₂H;
each n is independently 1 to 12;
X is O, NR or —CH₂—;
V is bond or C₁-C₆ alkyl;
one of R²⁴ and R²⁵ is selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; —C(O)—R²⁸, where R²⁸ is a Sugar unit of formula (XII) or (XIII); and the other of R²⁴ and R²⁵ is selected from H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-Cia carbocycle; optionally substituted heteroaryl; optionally substituted carbocycle; substituted —C₁-C₈ alkyl; substituted —C(O)—C₁-C₈ alkyl; a chelator; —C(O)—R²⁸, where R²⁸ is a Sugar unit of formula (XII) or (XIII); and polyethylene glycol, optionally having 1 to 24 ethylene glycol subunits; or —NR²⁴R²⁵ together from a C₃-C₈ heterocycle.

Embodiment 50. The Linker compound of any one of Embodiments 1-49, comprising a Polar group having a formula selected from:

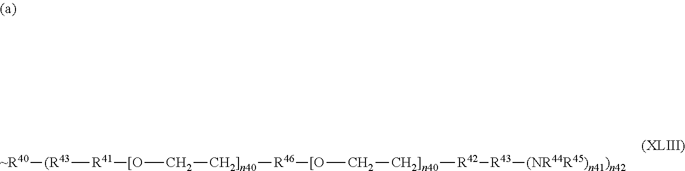

or a stereoisomer or salt thereof, wherein:
R⁴⁰ is an attachment group to site R^{b}, or to the enzyme-cleavable group;
R⁴¹ and R⁴² are each, independently, bond or C₁-C₆ alkylene;
each R⁴³ is, independently, selected from a bond, C₁-C₁₂ alkylene, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)—, —C(O)—C₁-C₁₂ alkylene-NH—, —NH—C(O)—NH—, —NH—C(O)—, —NH—C(O)—C₁-C₁₂ alkylene, —C(O)—NH—C₁-C₁₂ alkylene, C₁-C₁₂alkylene-NH—C(O)—, -heteroarylene, heteroaryl-C₁-C₁₂ alkylene, heteroaryl-C₁-C₁₂ alkylene-C(O)—, or —C(O)NR⁴⁶R⁴⁷, wherein one of R⁴⁶ and R⁴⁷ is H or C₁-C₁₂ alkylene and the other is C₁-C₁₂ alkylene;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate;
each R⁴⁶ is independently selected from —NR⁵⁰—, —NR⁵⁰—C₁-C₆alkylene-NR⁵⁰—, —NR⁵⁰—C(O)—NR⁵⁰—S(O)₂—NR⁵⁰— or —NR⁵⁰—C(O)—C₁₋₆alkylene-; each R⁵⁰ is independently selected from H, C₁-C₆ alkyl, or polyhydroxyl group;
n40 is 2 to 26;
n41 is 1 to 6; and
n42 is 1 to 6;

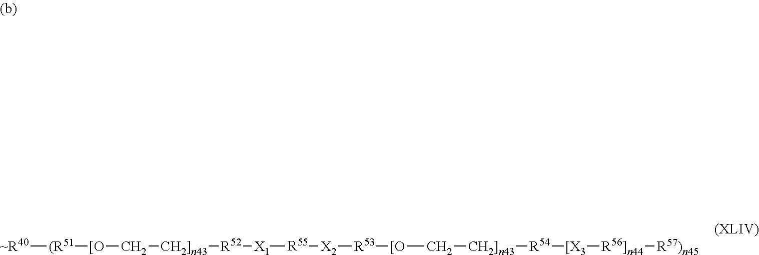

or a stereoisomer or salt thereof, wherein:
R⁴⁰ is an attachment group to site R^{b}, or to the enzyme-cleavable group;
R⁵¹, R⁵², R⁵³ and R⁵⁴ are each, independently, bond or C₁-C₆ alkylene;
X₁, X₂ and X₃ are each independently —NR^{N}—C(O)— or —C(O)—NR^{N}—;
each R^{N} independently represent H, C₁-C₆ alkyl, or polyhydroxyl group;
R⁵⁵ and R⁵⁶ each independently represent a bivalent polyhydroxyl group;
R⁵⁷ is H, OH or C₁-C₆ alkyl;
each n43 is independently 0 to 26, with the proviso that at least one n43 is 1 to 26;
n44 is 0 to 10; and
n45 is 1 or 2; or

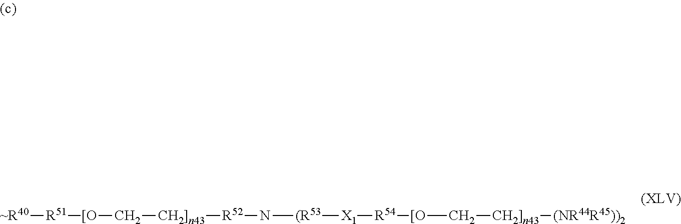

or a stereoisomer or salt thereof, wherein:
R⁴⁰ is an attachment group to site R^{b}, or to the enzyme-cleavable group;
R⁵¹, R⁵³ and R⁵⁴ are each, independently, bond or optionally-substituted C₁-C₆ alkylene;
R⁵² is a bond, C₁-C₆ alkylene, —C(O)— or —O—C(O)—;
each X₁ is independently —NR^{N}—C(O)— or —C(O)—NR^{N}—;
each R^{N} independently represent H, C₁-C₆ alkyl, or polyhydroxyl group;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate; and
each n43 is independently 2 to 26.

Embodiment 51. The Linker compound of any one of Embodiments 1-50, comprising a Polar group having one of the following structures prior to attachment to the enzyme-cleavable group and/or to the Linker Unit:

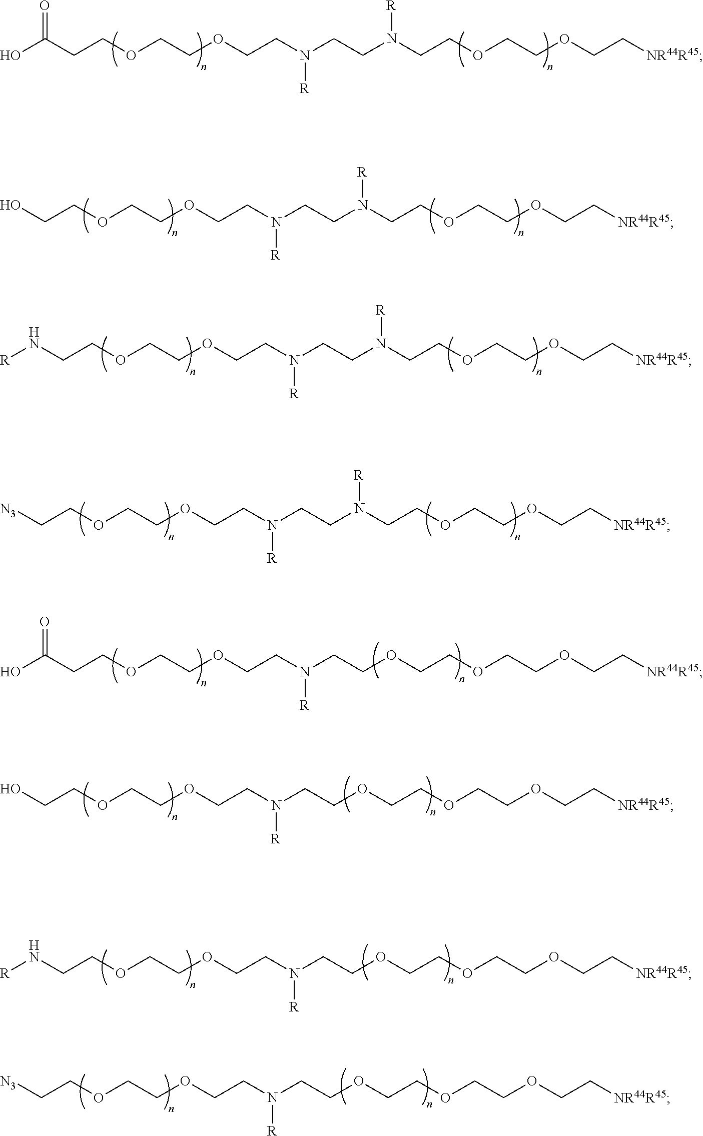

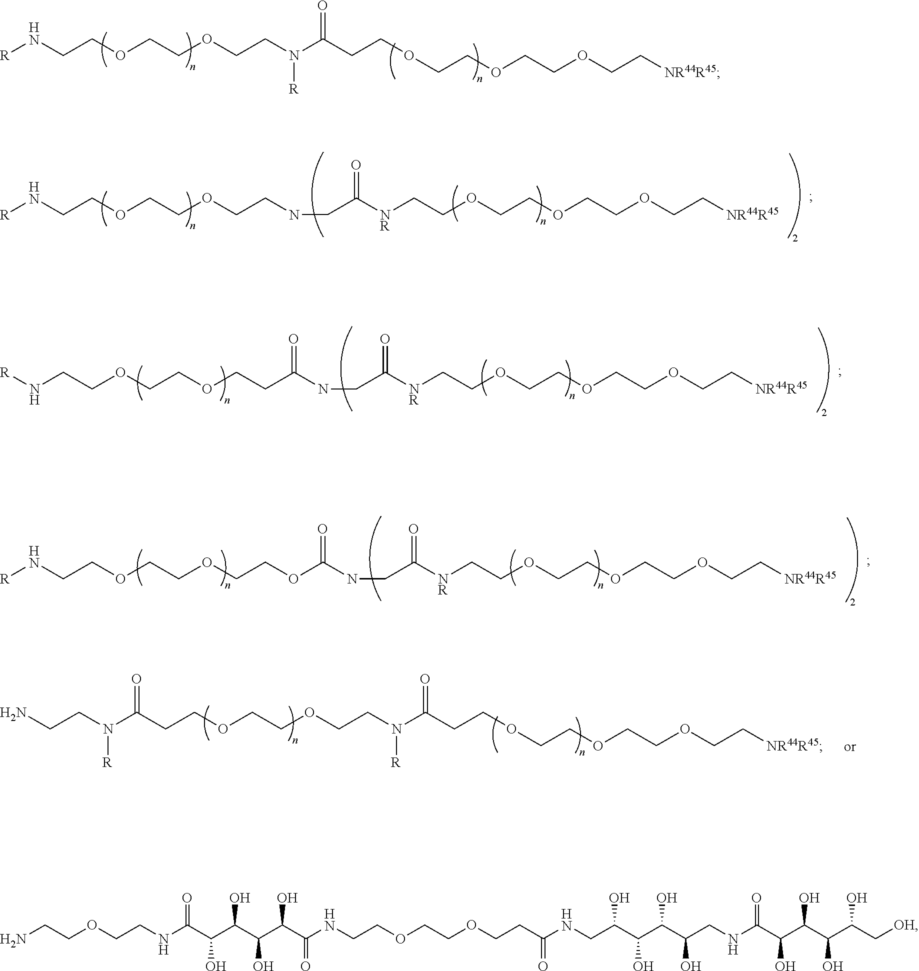

wherein:
each R is independently H, alkyl or polyhydroxyl group;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate; and
each n is independently 1 to 12.

Embodiment 52. The Linker compound of any one of Embodiments 1-51, comprising a Polar group having a formula selected from:

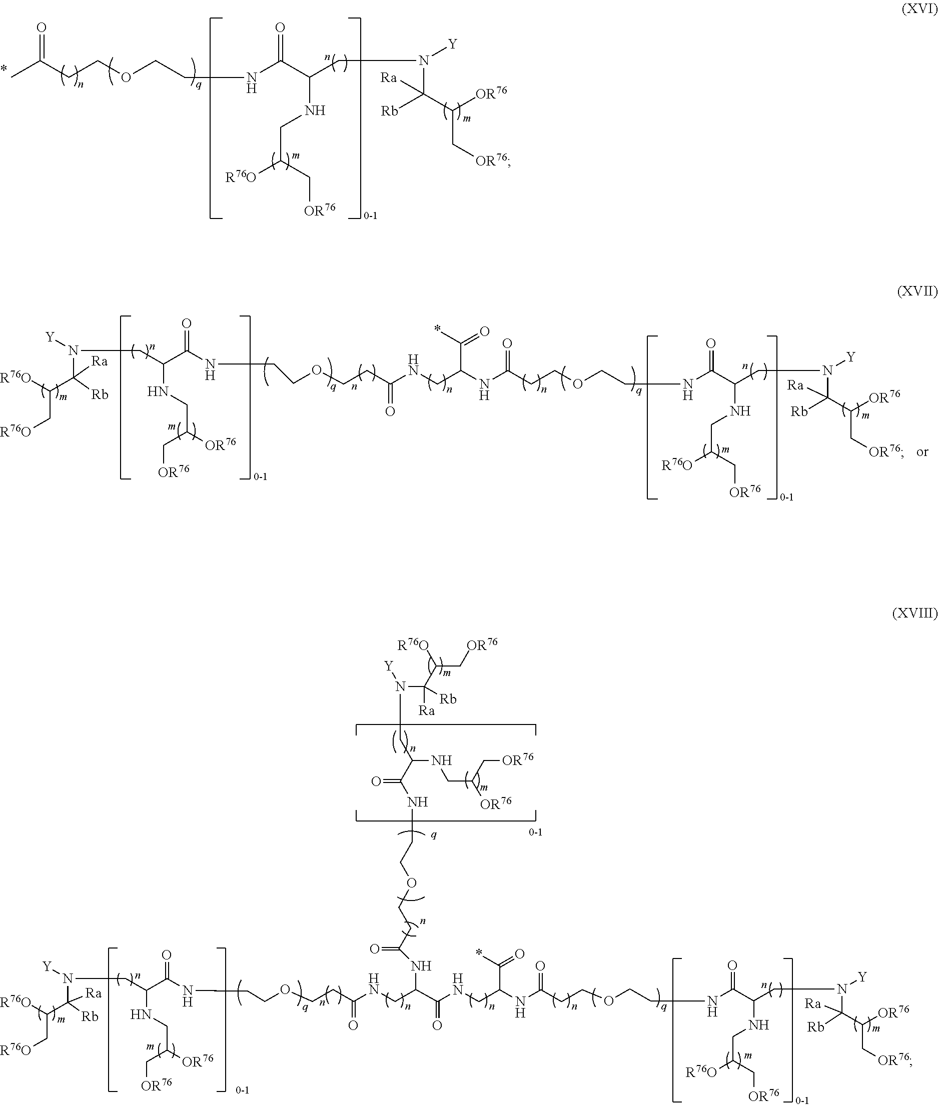

or a stereoisomer or salt thereof, wherein:
   each Y is independently R⁷⁶ or

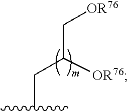

each R⁷⁶ is independently H, acetyl, —P(═O)(OH)₂, or —(CH₂)v-O—S(═O)₂(OH);
each Rₐ and R_{b} is independently H or Rₐ and R_{b} are taken together with the carbon to which they are attached to form an oxo group;
each q is independently 2-26;
each m is independently 1 to 4;
each n is independently 1 to 4;
each v is independently 1 to 6; and
each * is an attachment site to R^{b}, or to the enzyme-cleavable group.

Embodiment 53. The Linker compound of any one of Embodiments 1-51, comprising a Polar group having a formula selected from:

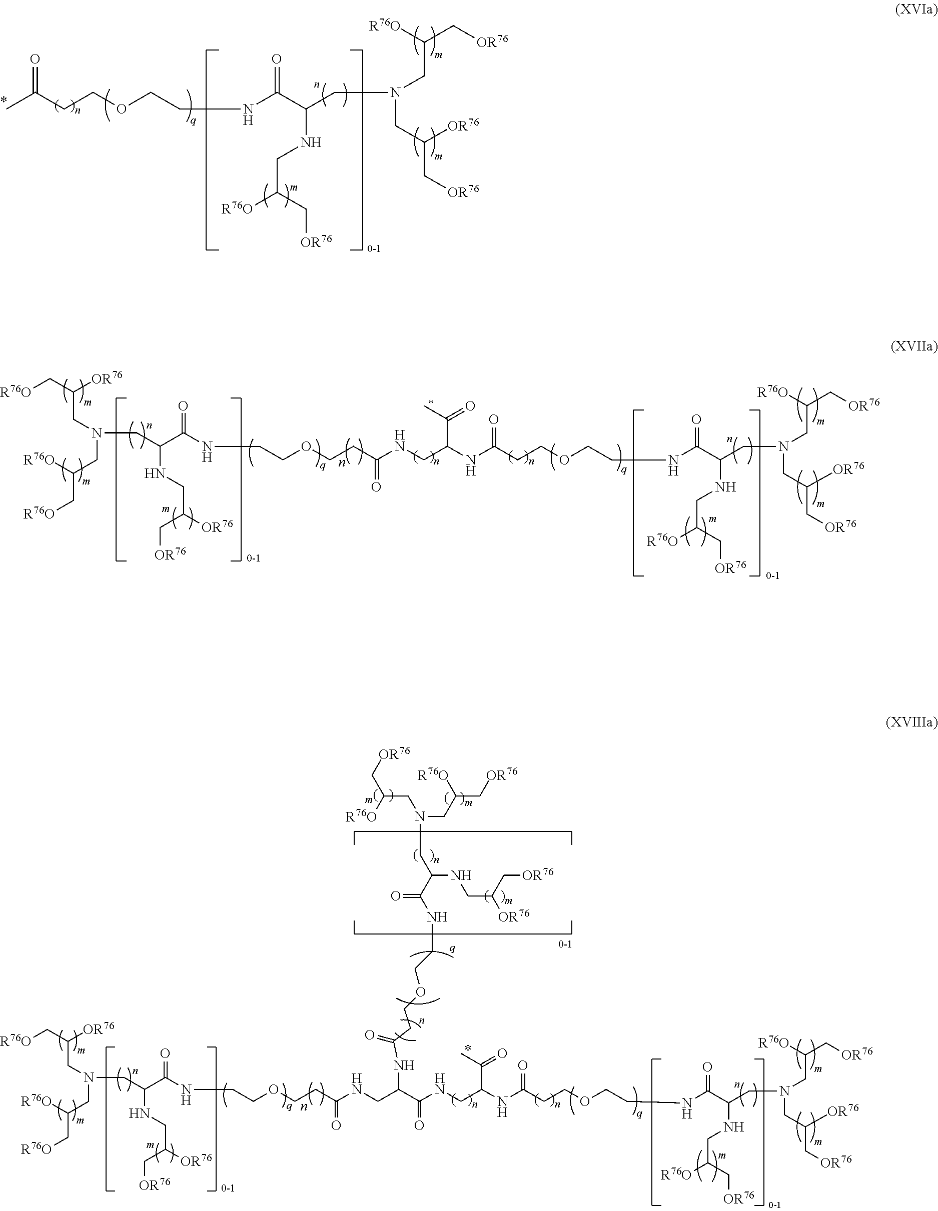

or a stereoisomer or salt thereof, wherein:
each R⁷⁶ is independently H, acetyl, —P(═O)(OH)₂, or —(CH₂)ᵥS(═O)₂(OH);
each q is independently 2-26;
each m is independently 1 to 4;
each n is independently 1 to 4;
each v is independently 1 to 6; and
each * is an attachment site to R^{b}, or to the enzyme-cleavable group.

Embodiment 54. The Linker compound of any one of Embodiments 1-51, comprising a Polar group having a formula selected from:

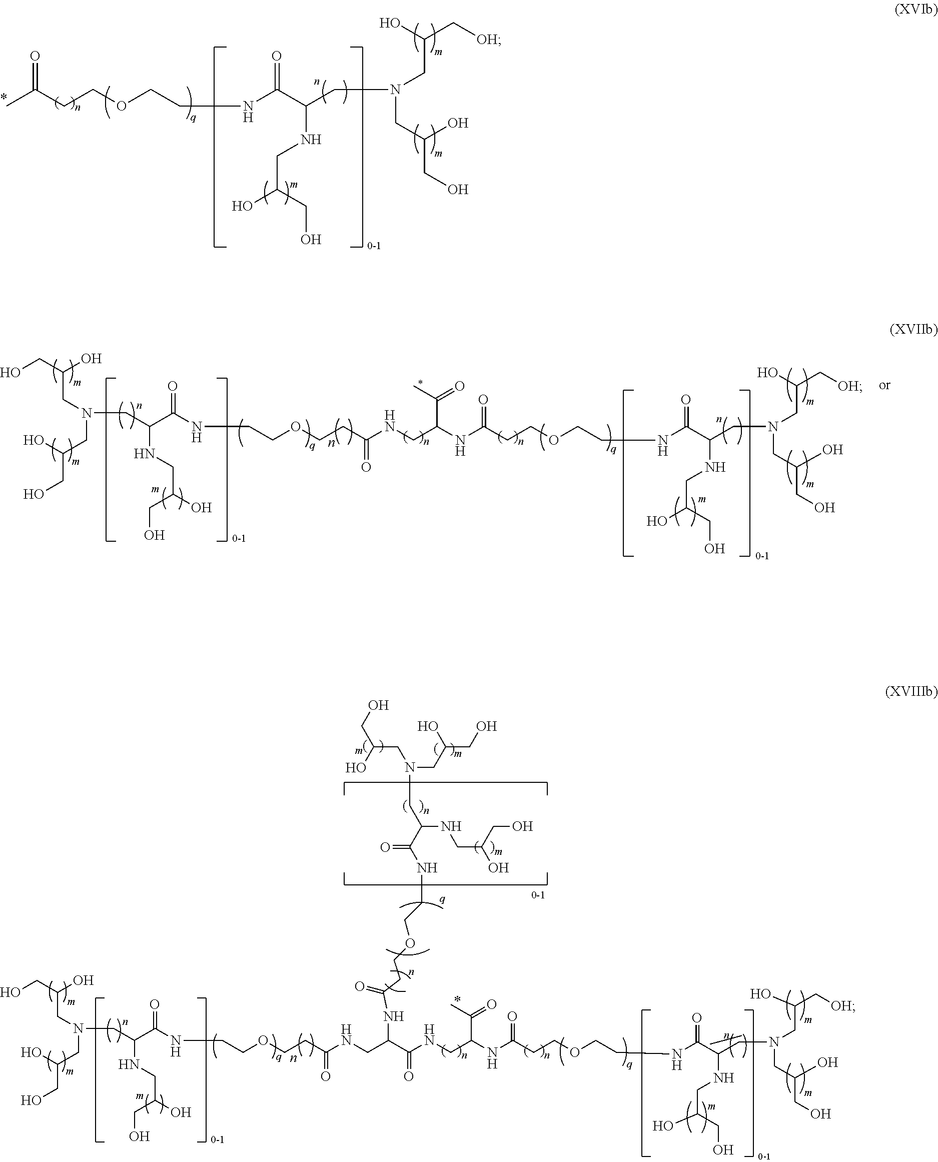

or a stereoisomer or salt thereof, wherein:
each q is independently 2-26;
each m is independently 1 to 4;
each n is independently 1 to 4; and
each * is an attachment site to R^{b}, or to the enzyme-cleavable group.

Embodiment 55. The Linker compound of Embodiment 52, wherein Y is R⁷⁶.

Embodiment 56. The Linker compound of Embodiment 52, wherein Y is

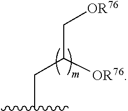

Embodiment 57. The Linker compound of Embodiment 52, wherein each Rₐ and R_{b} is independently H.

Embodiment 58. The Linker compound of Embodiment 52, wherein Rₐ and R_{b} are taken together with the carbon to which they are attached to form an oxo group.

Embodiment 59. The Linker compound of one of Embodiments 50-52, wherein q is 10-20.

Embodiment 60. The Linker compound of any one of Embodiments 52-54, wherein q is 12.

Embodiment 61. The Linker compound of any one of Embodiments 1-60, comprising a Polar group selected from the following, or a stereoisomer or salt thereof:

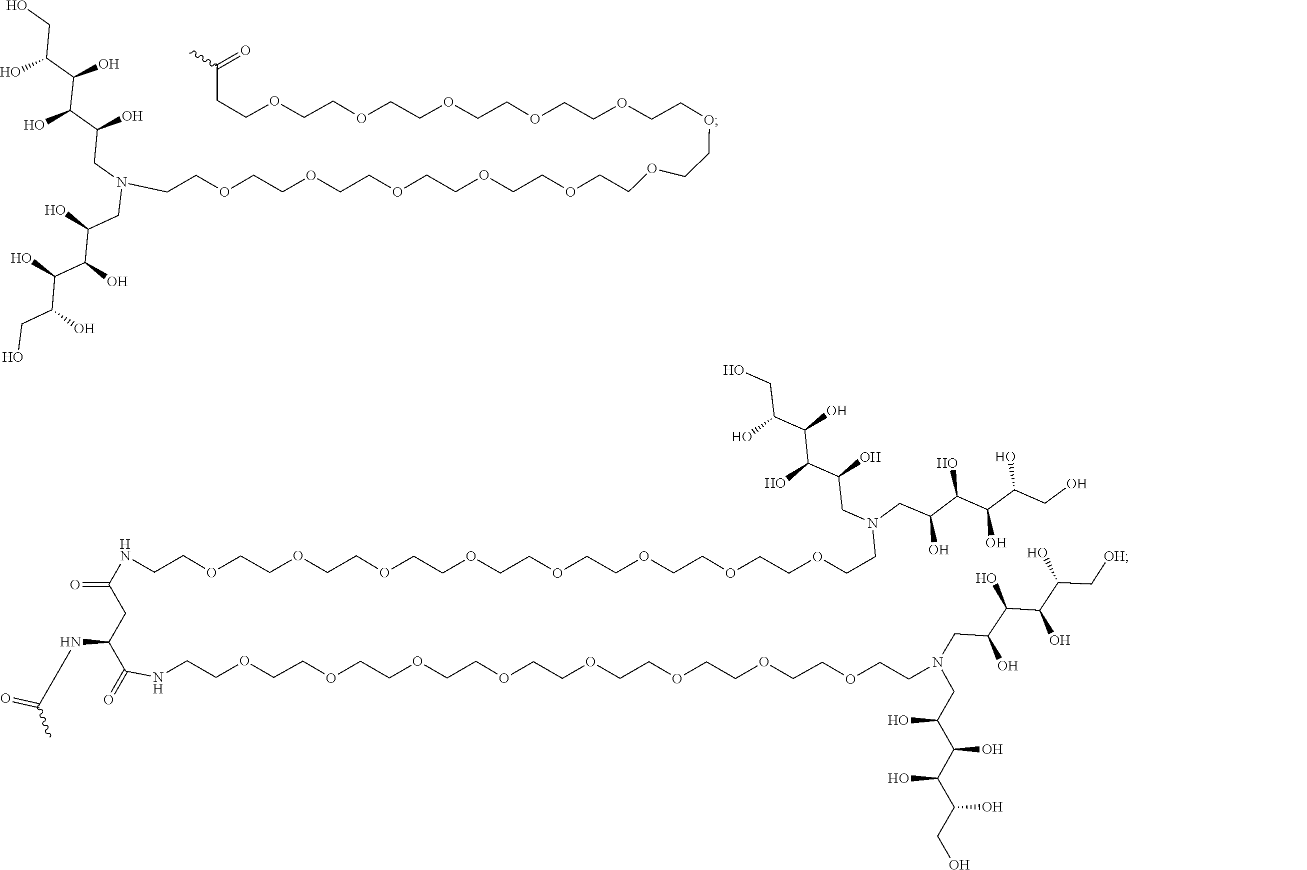

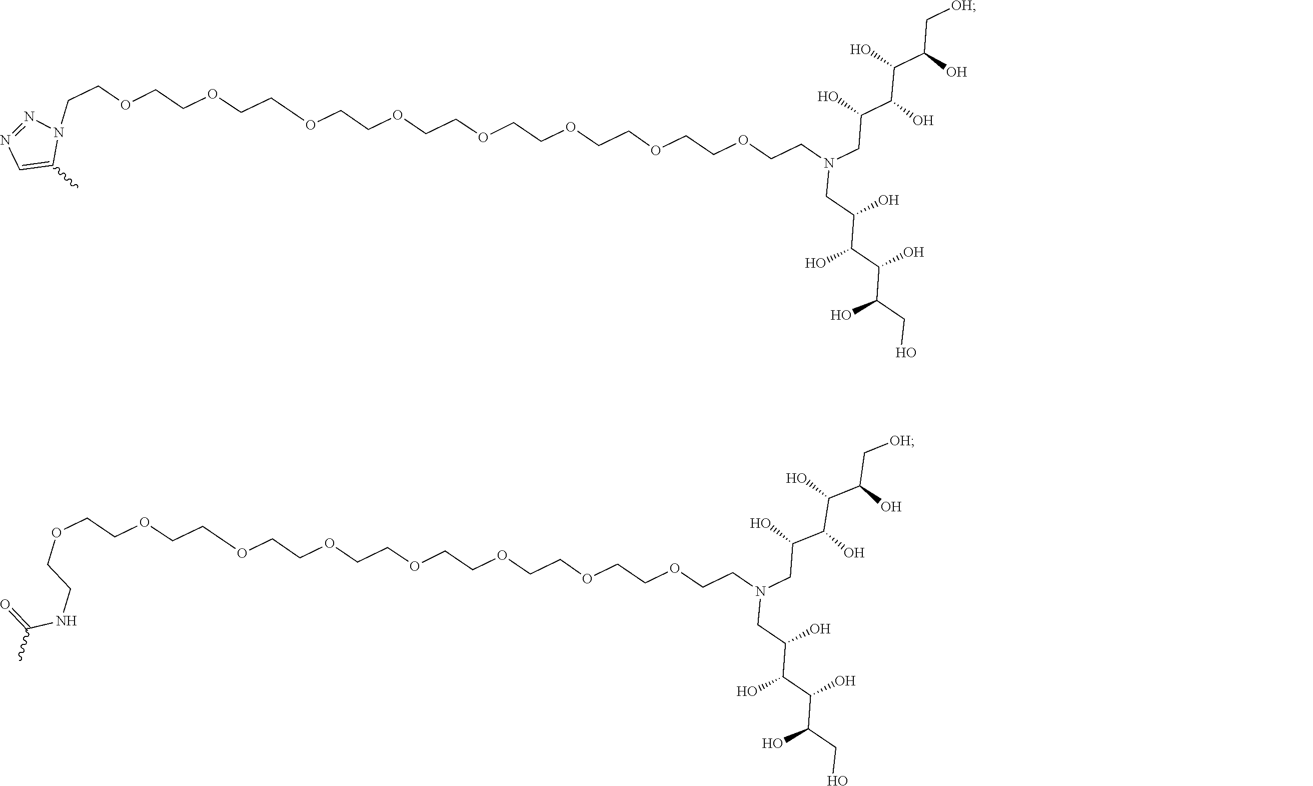

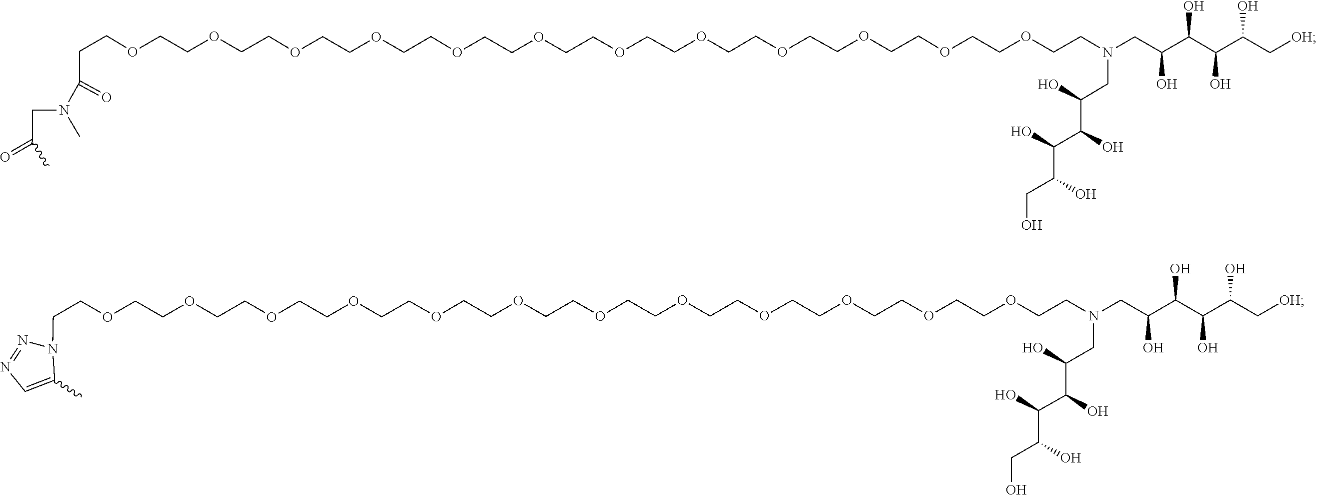

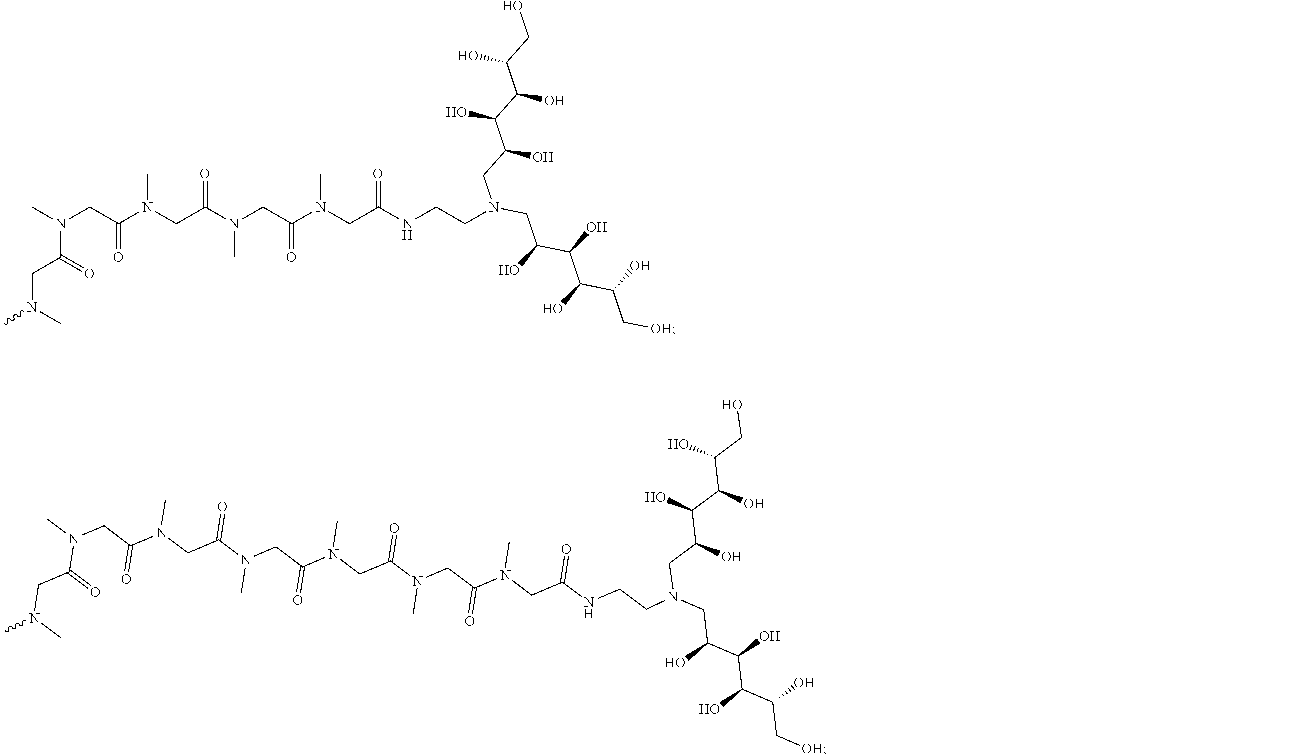

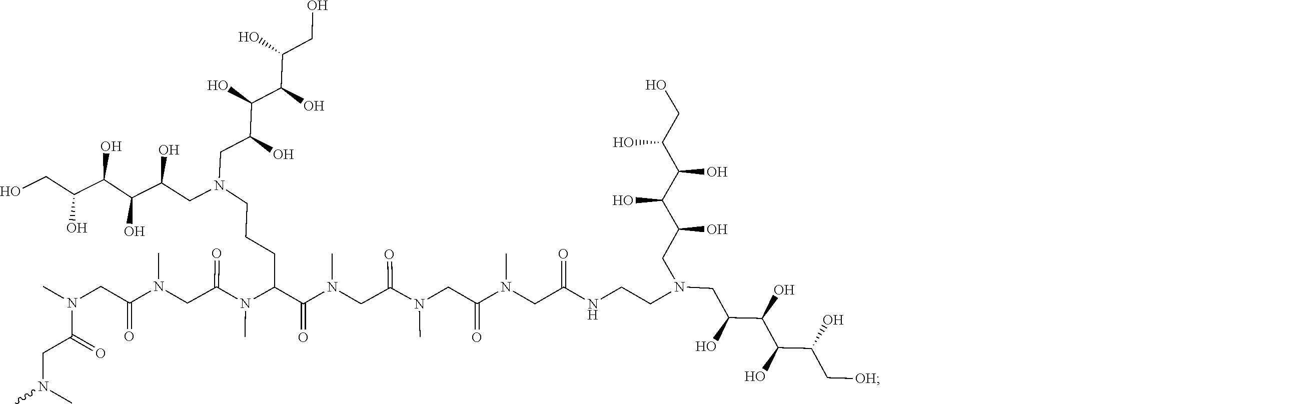

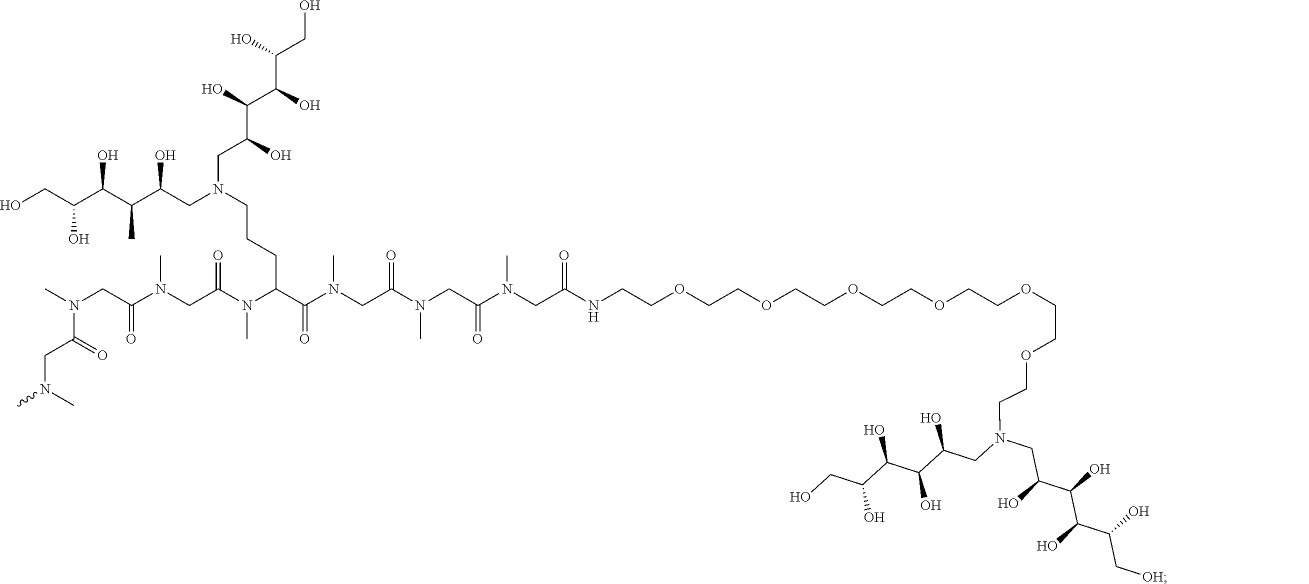

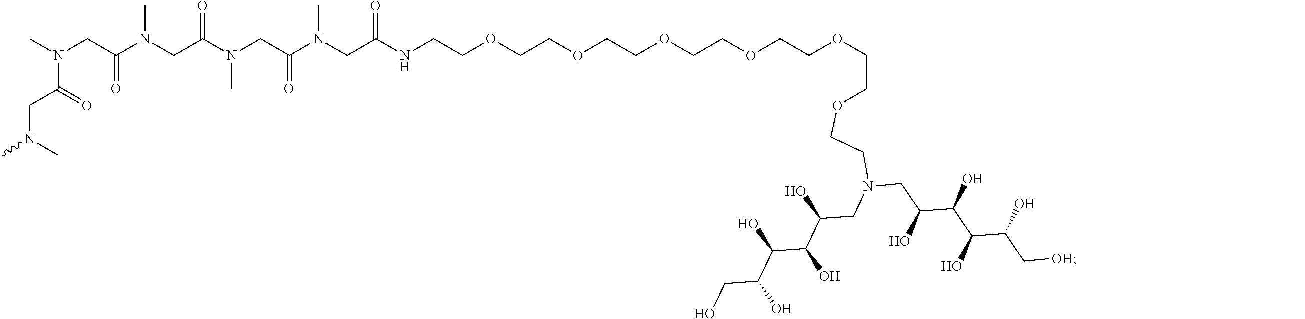

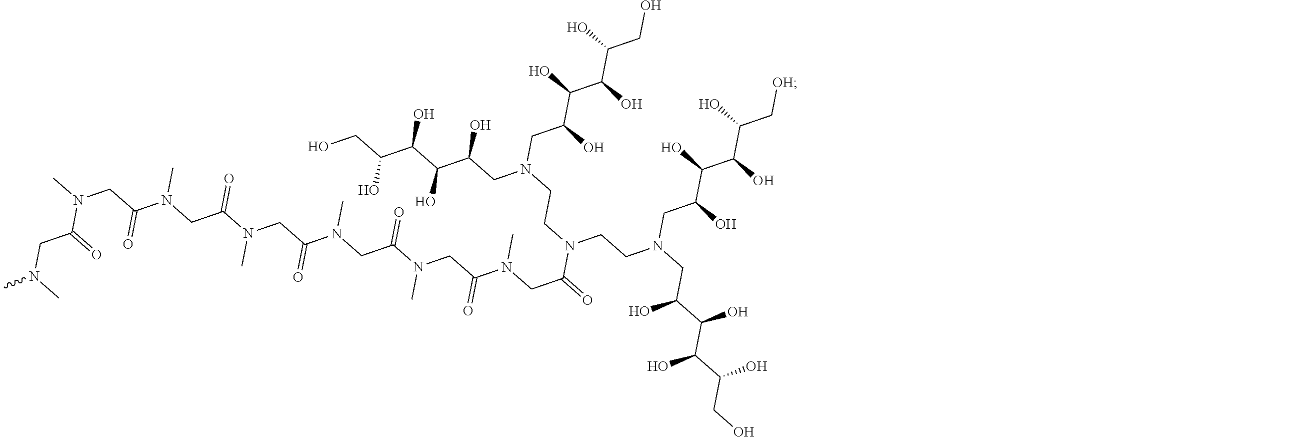

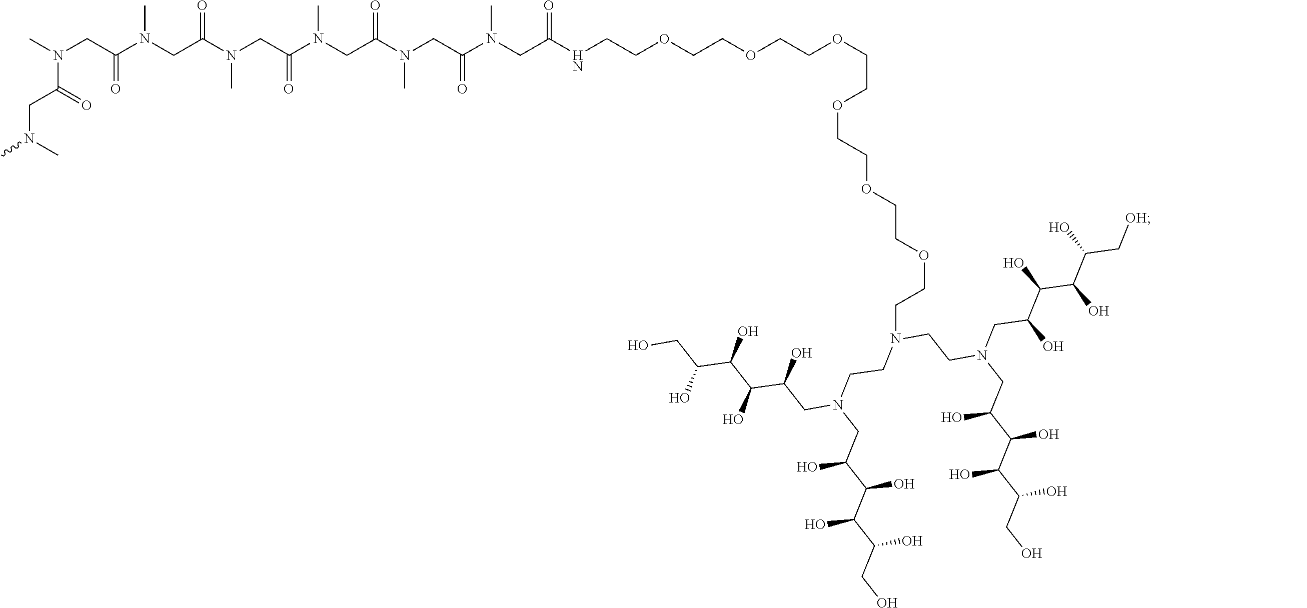

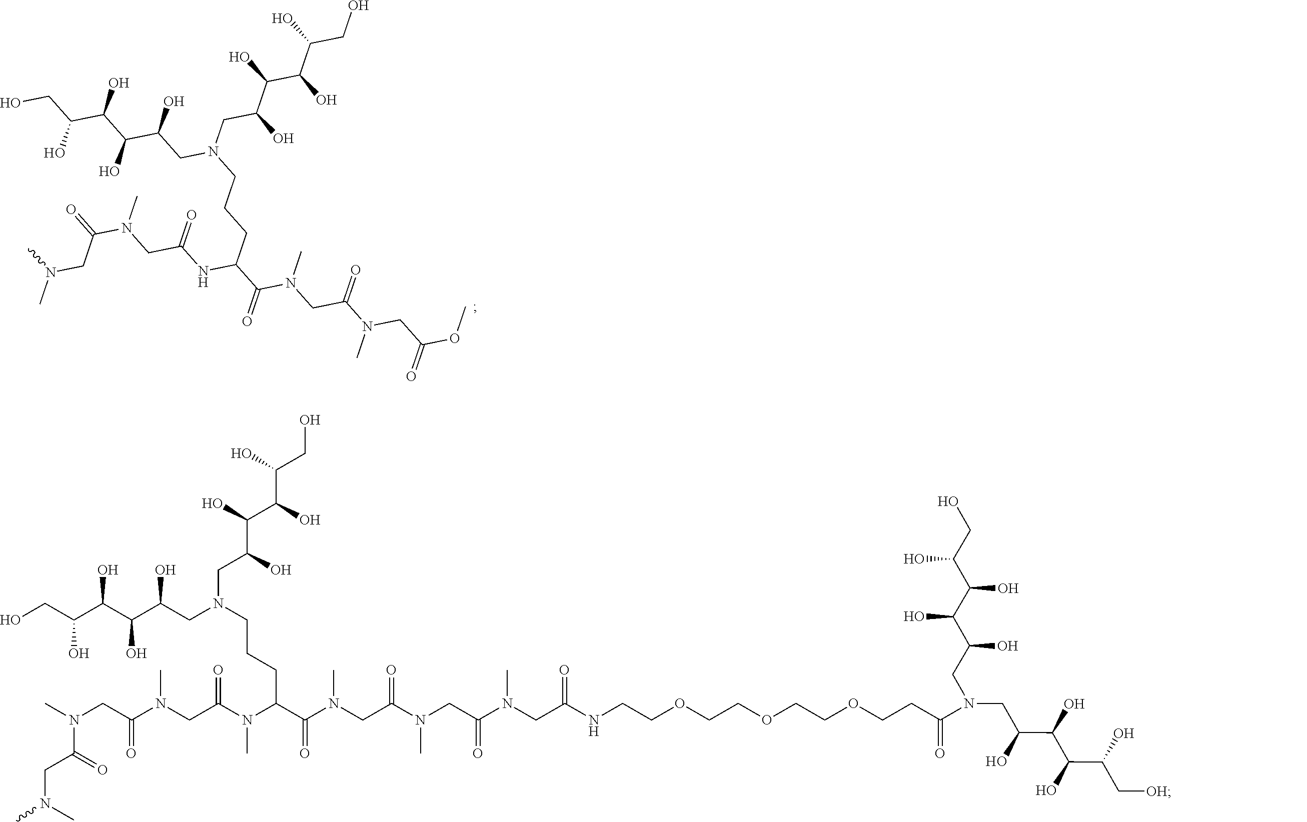

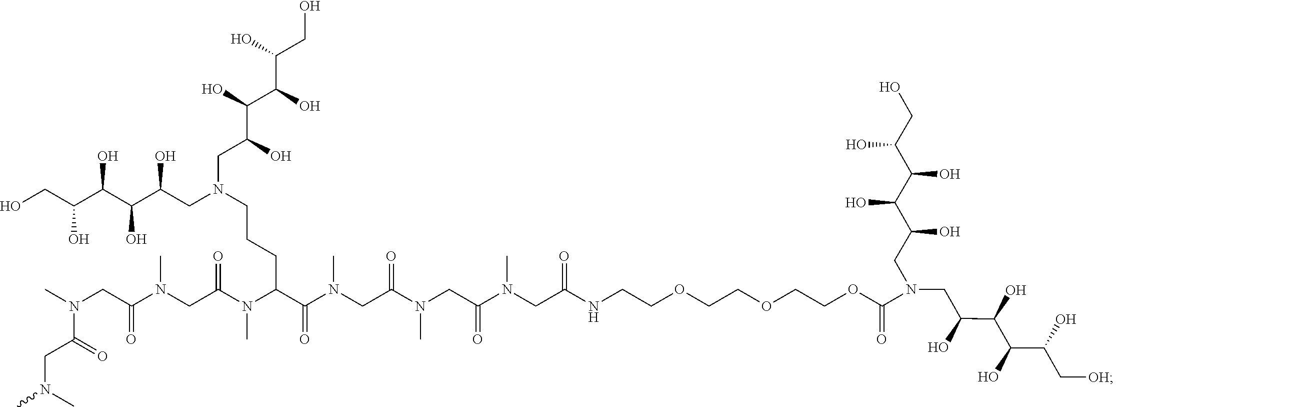

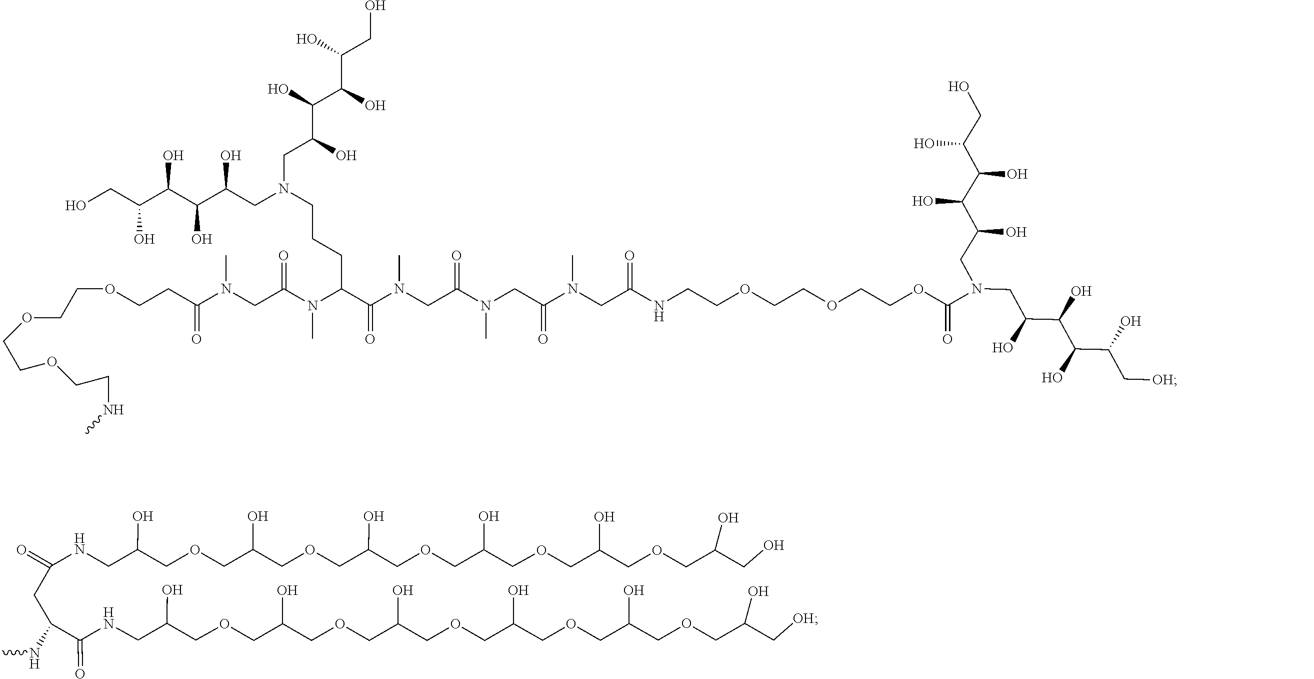

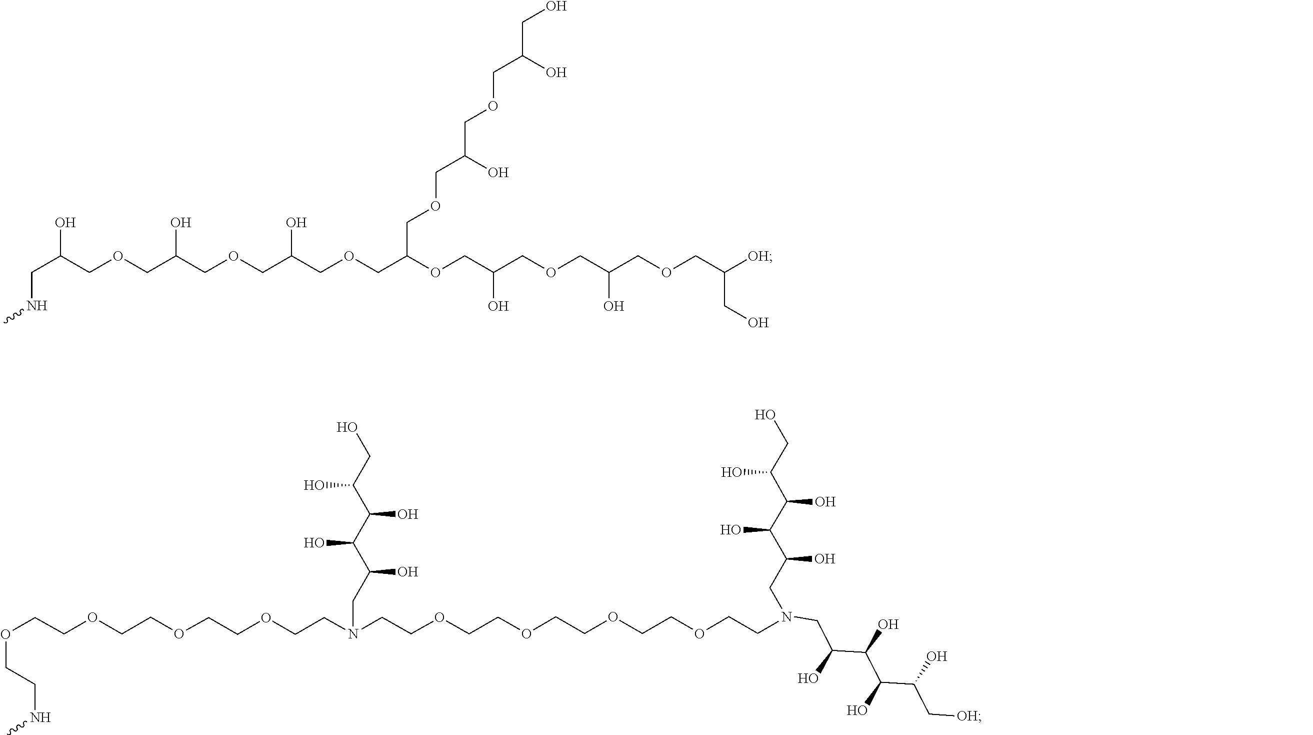

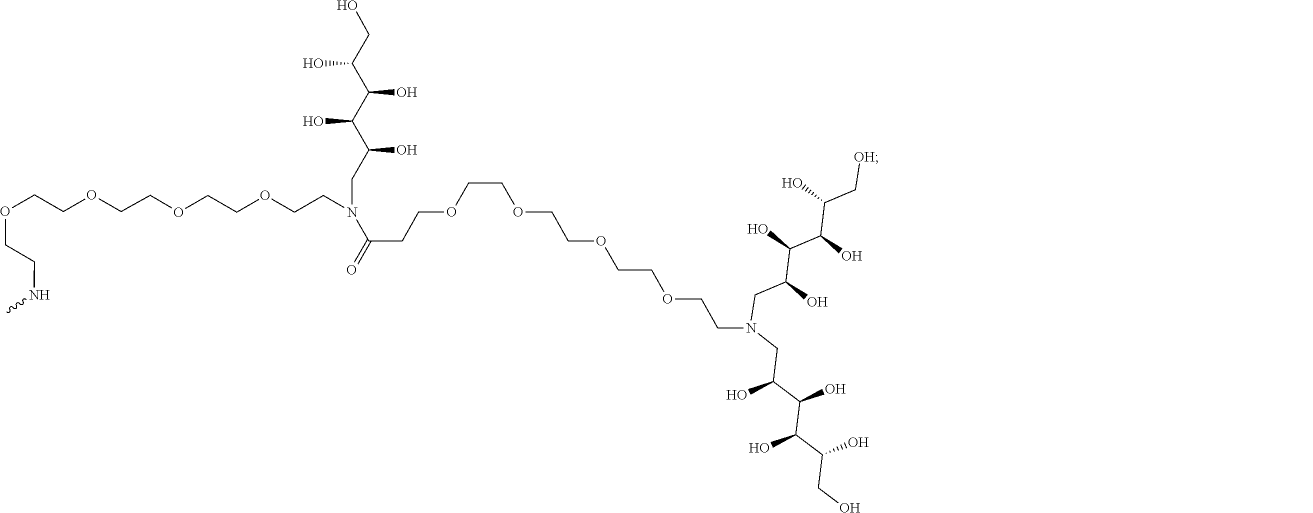

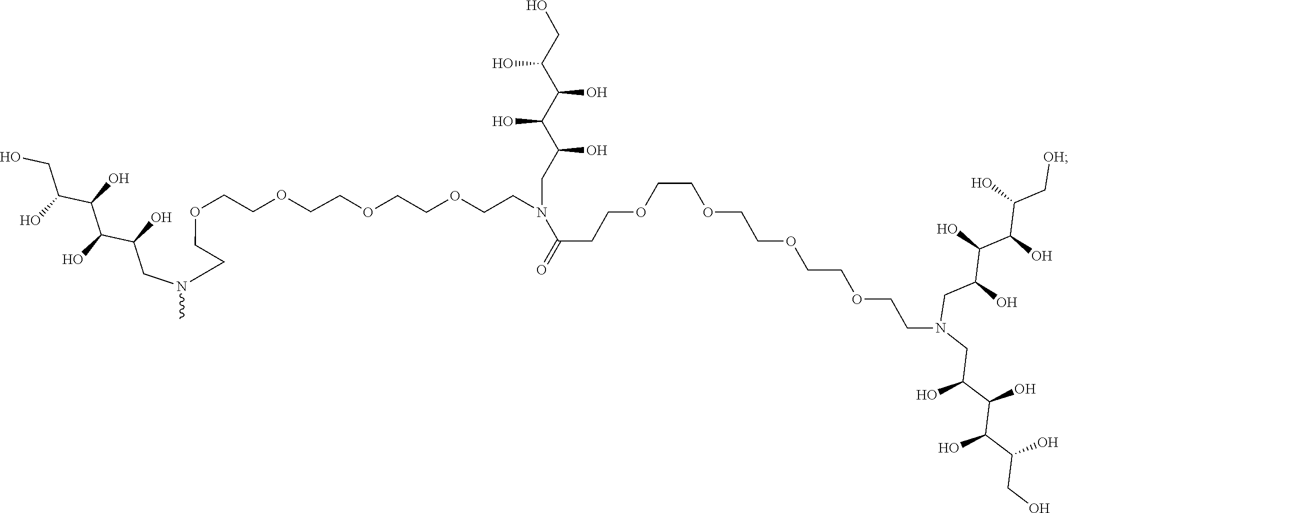

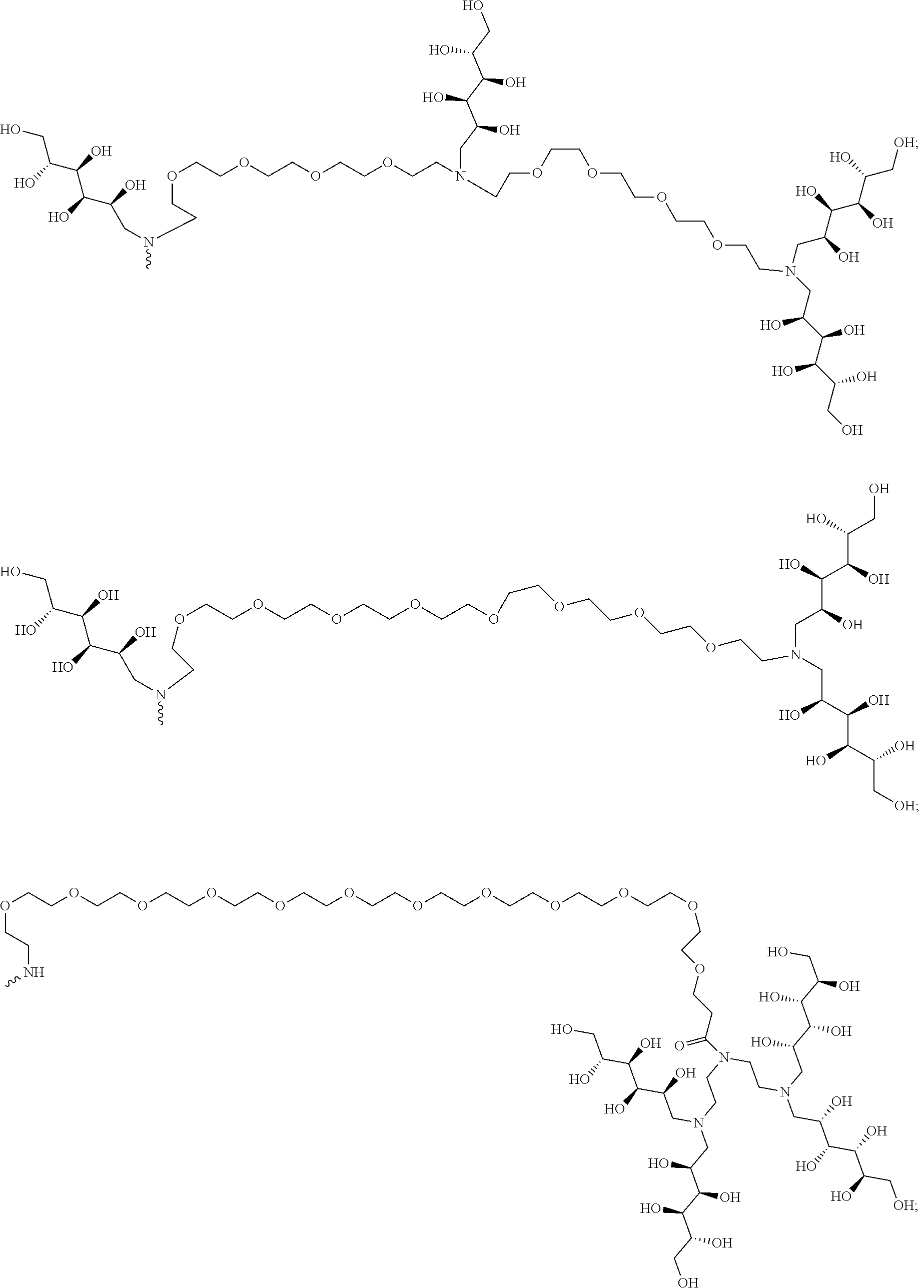

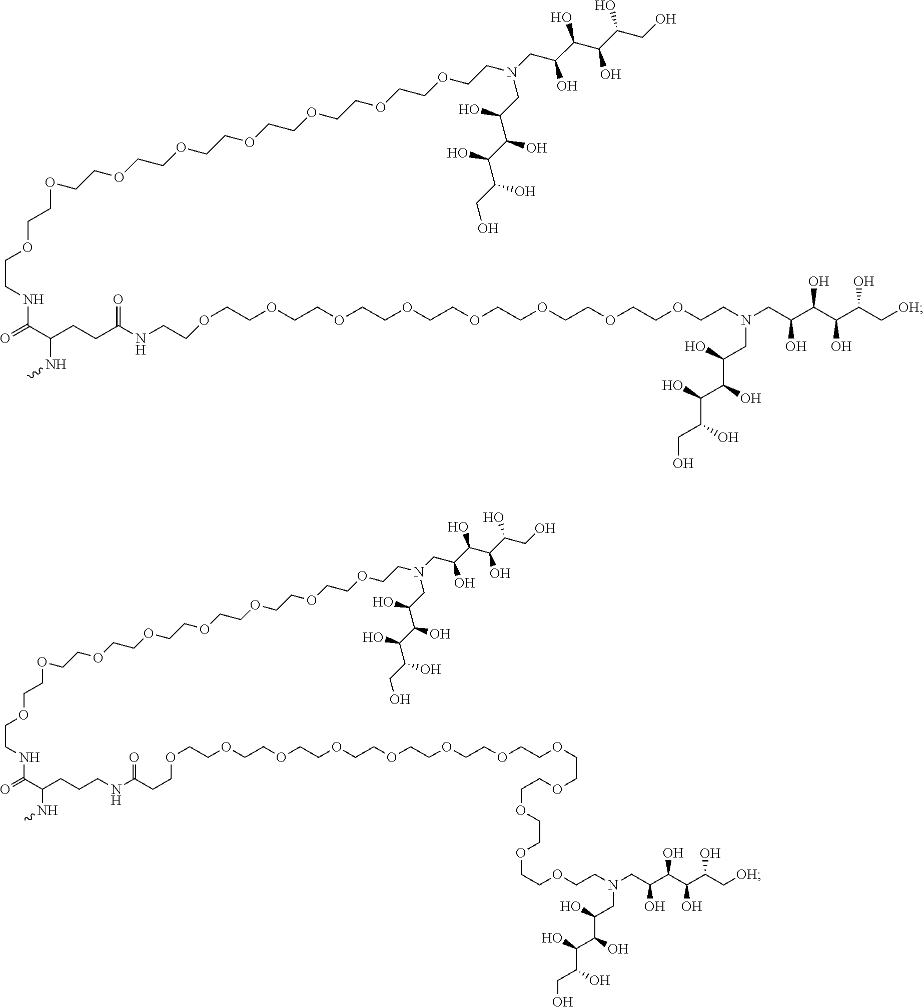

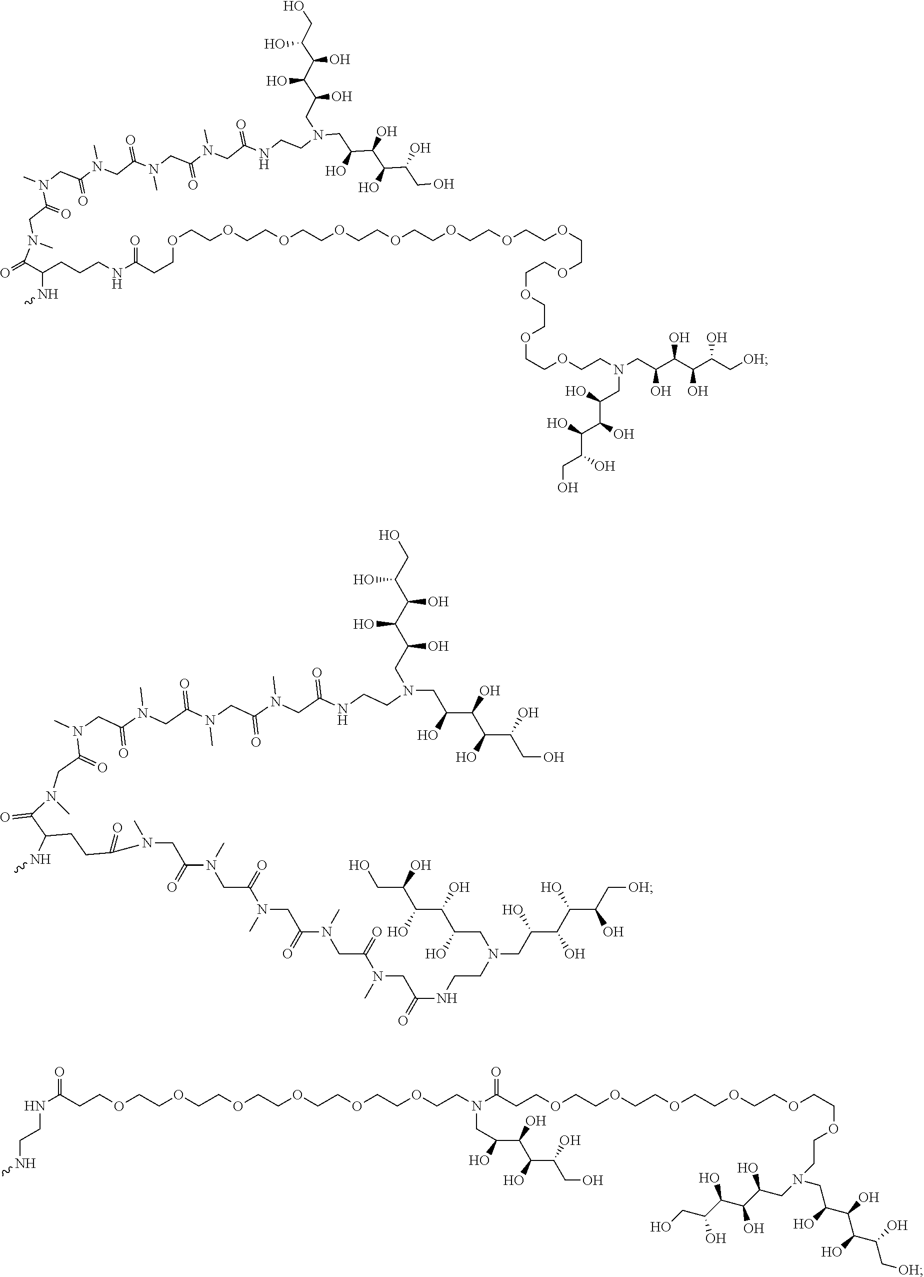

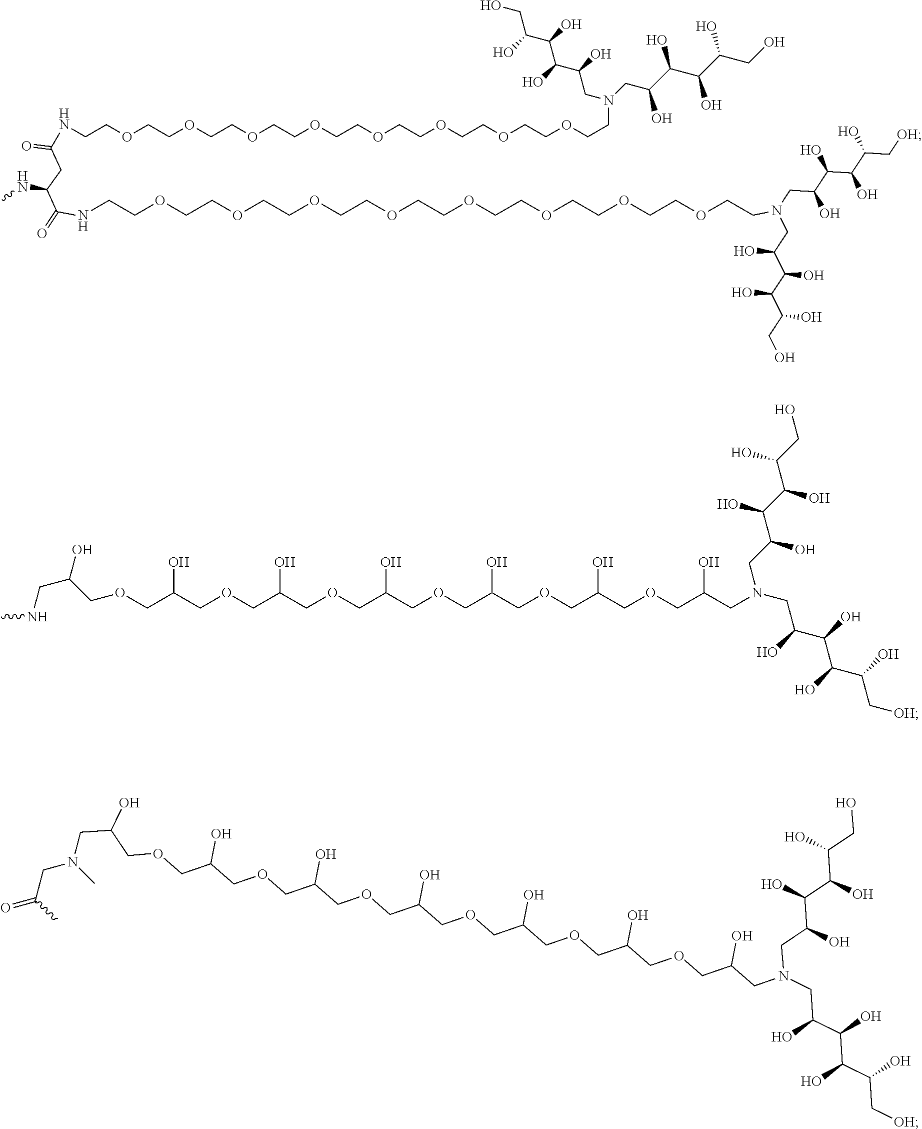

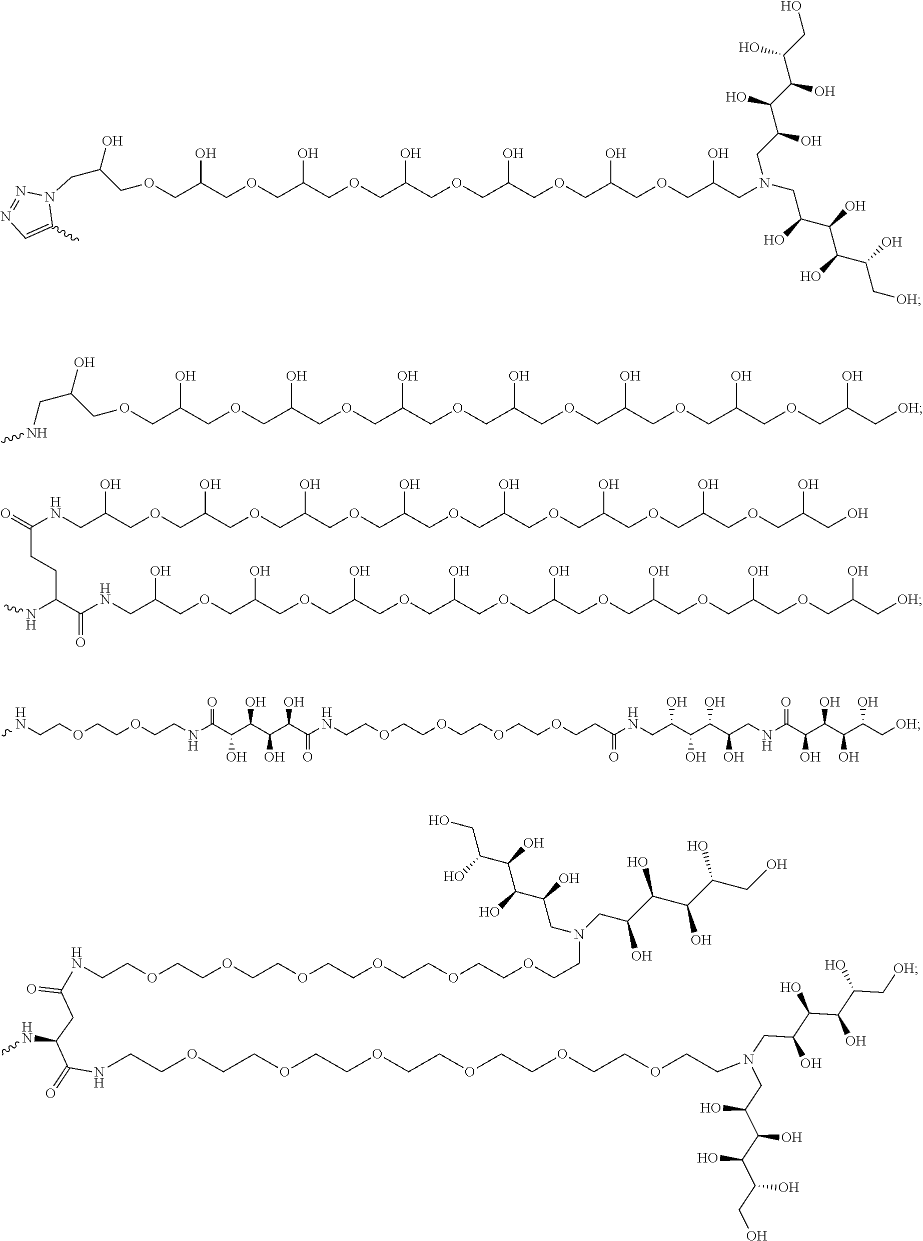

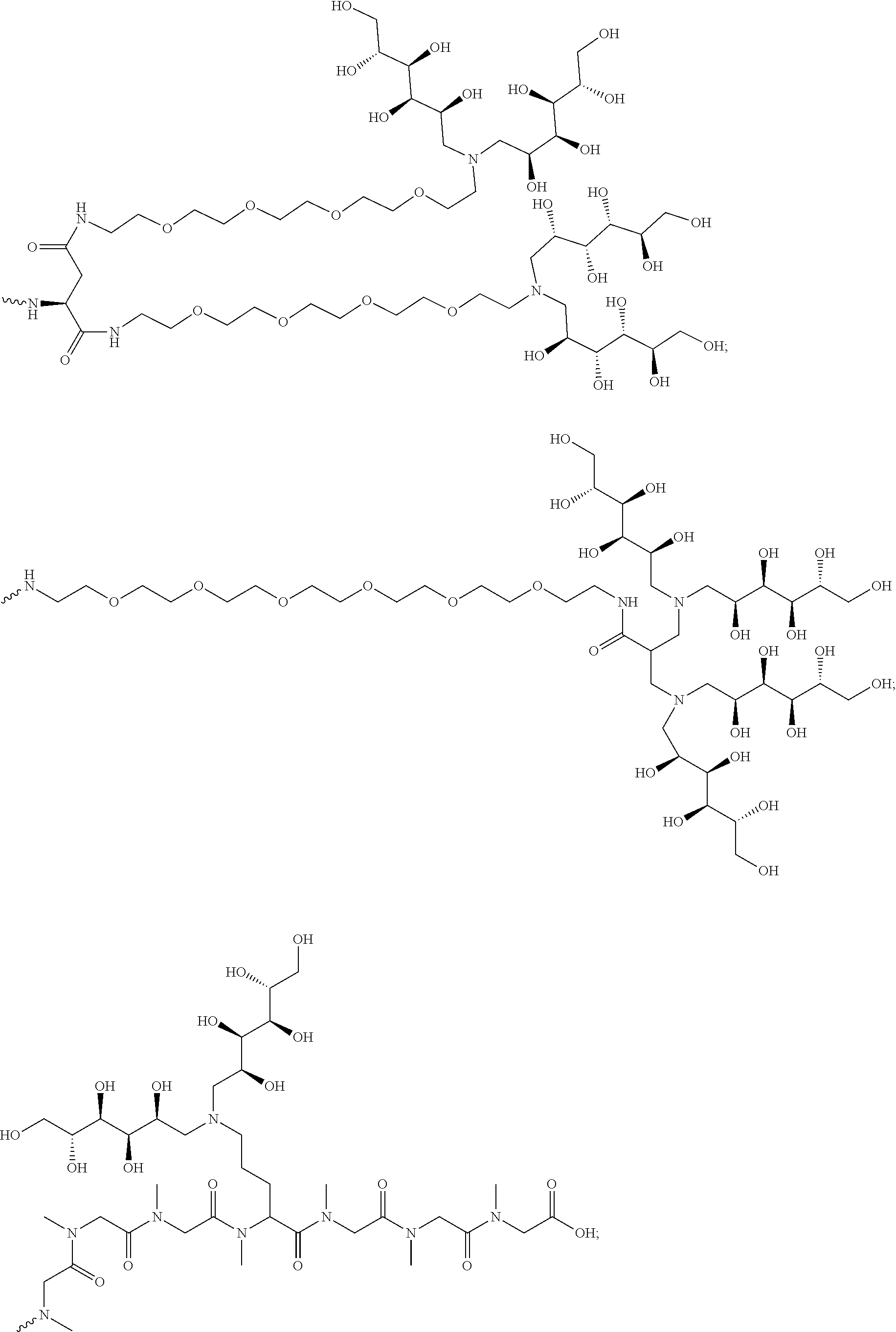

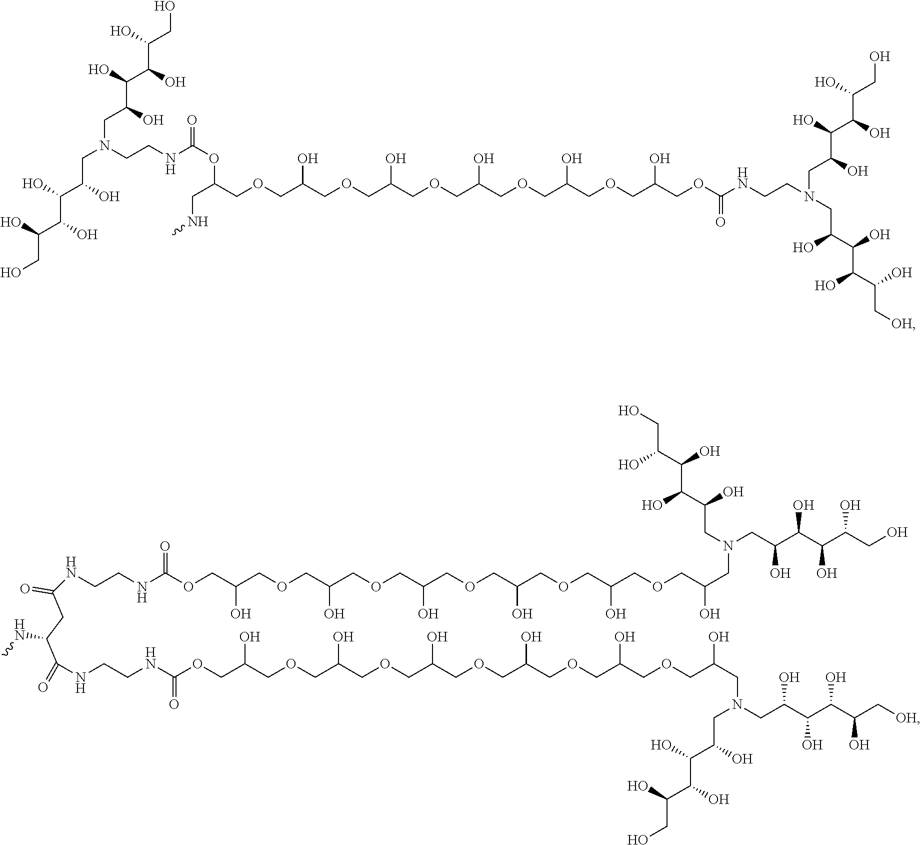

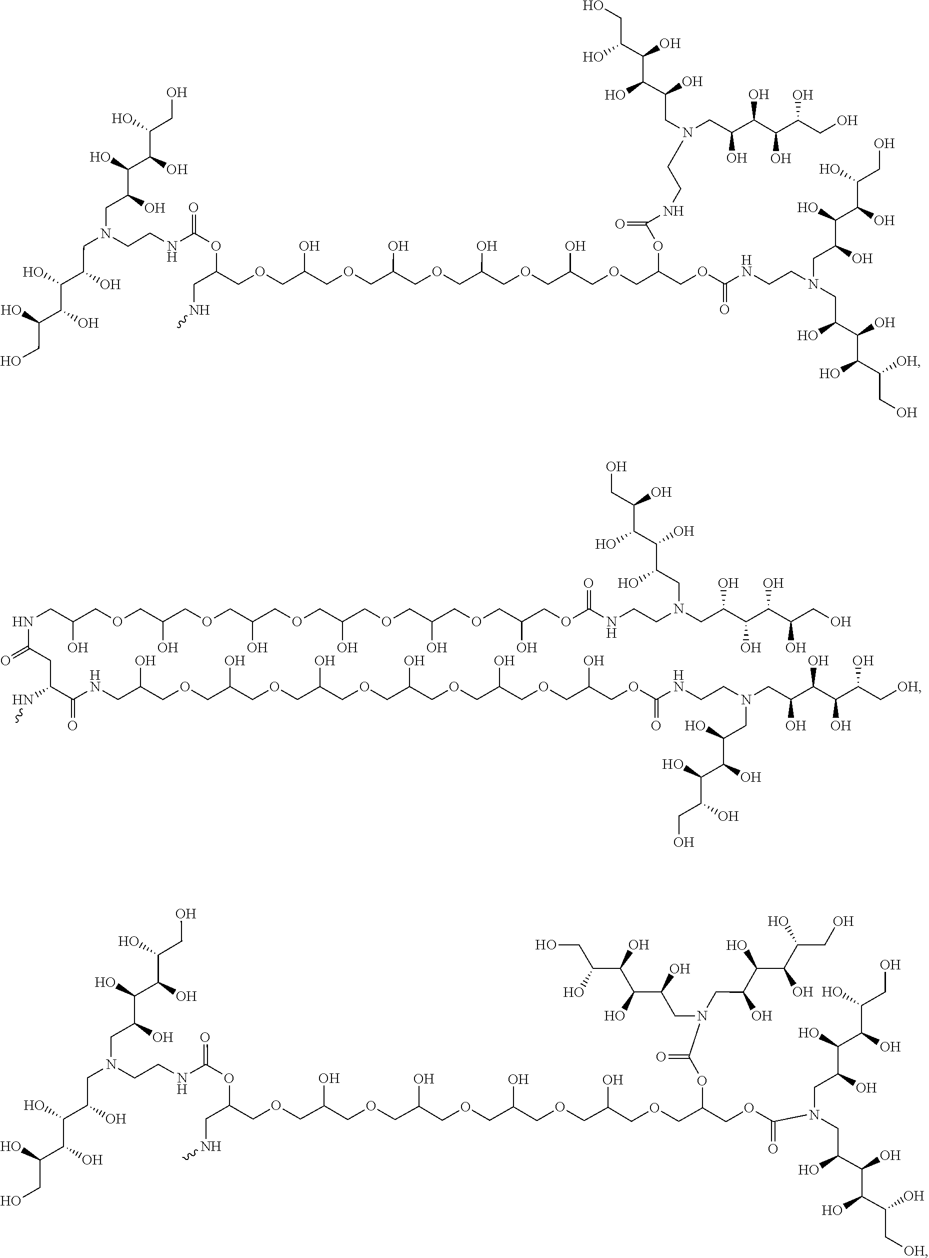

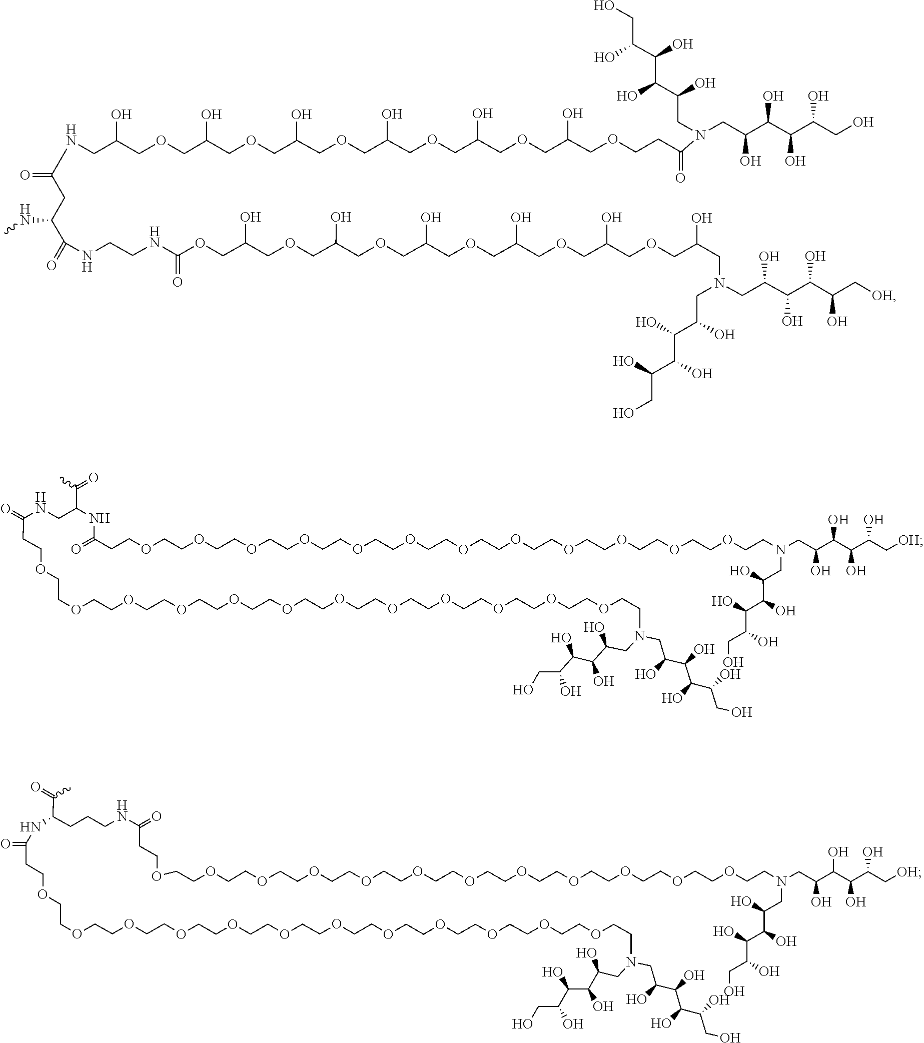

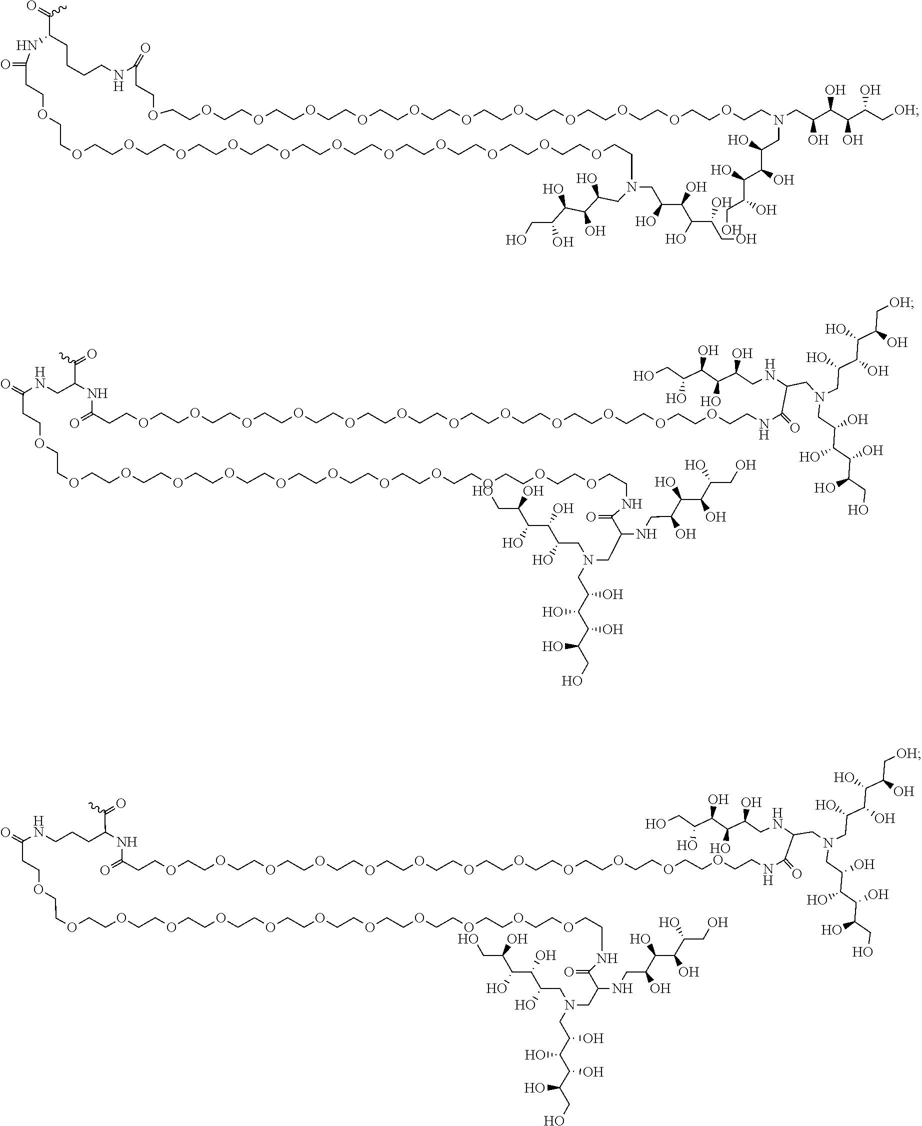

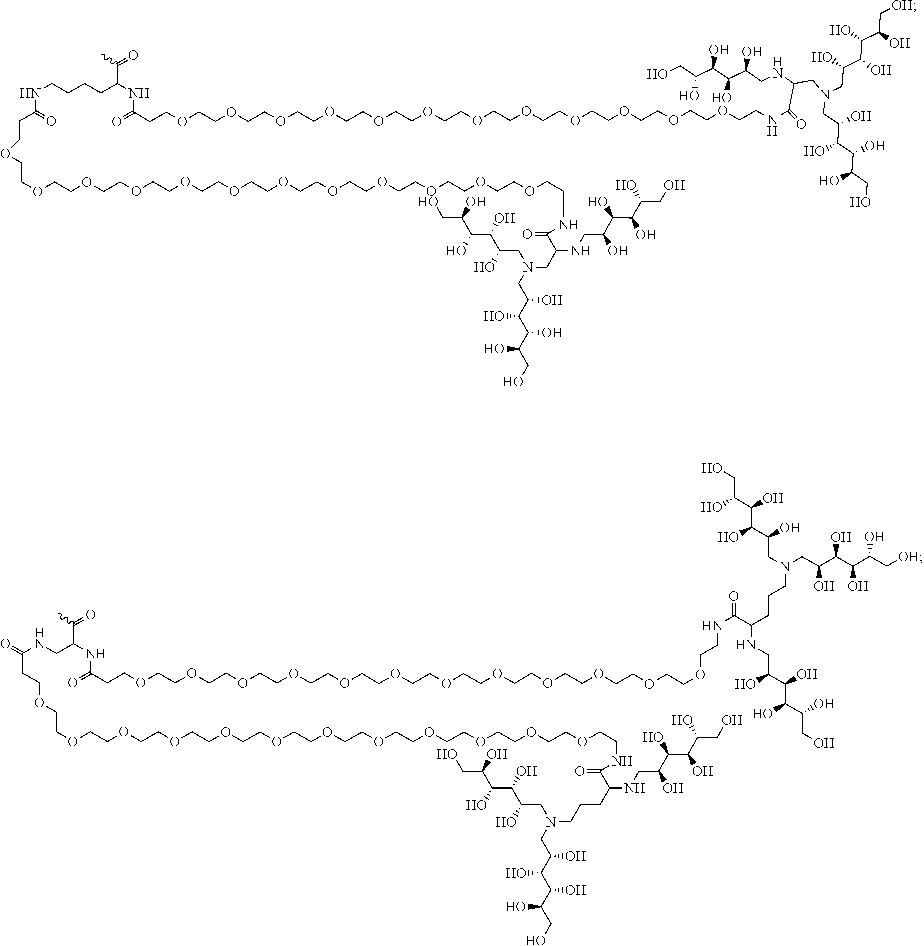

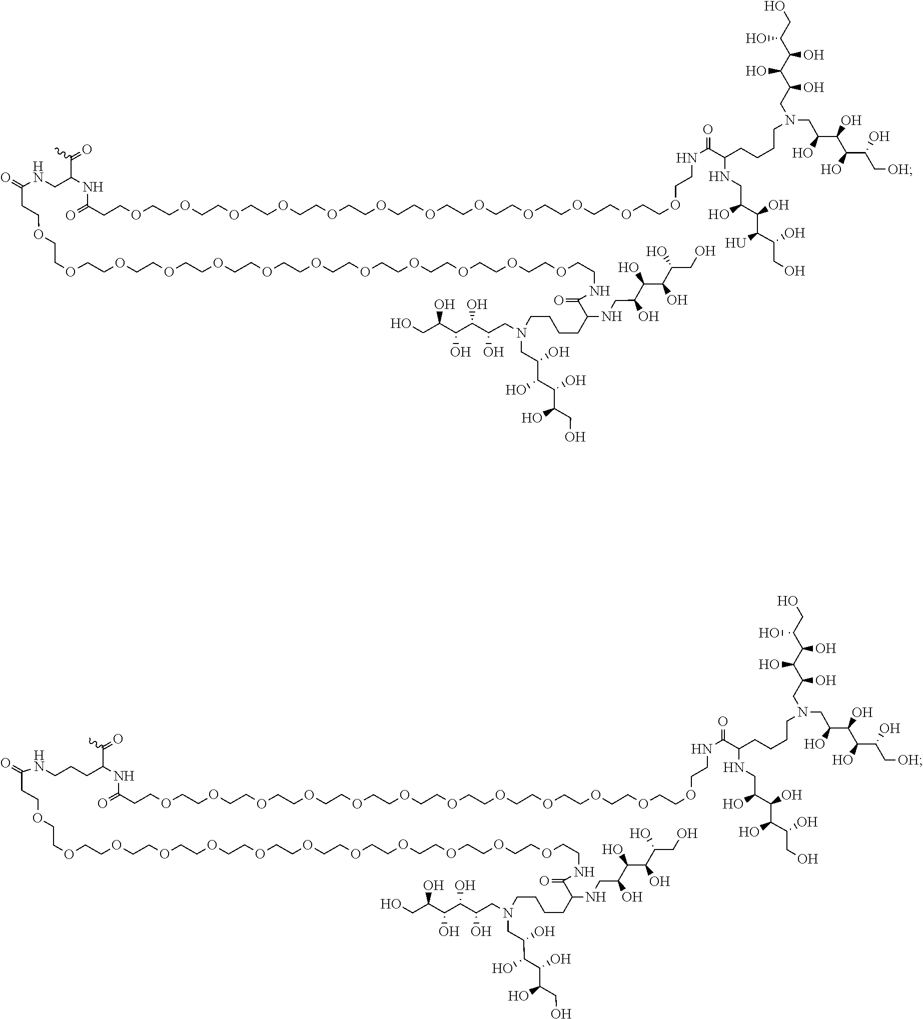

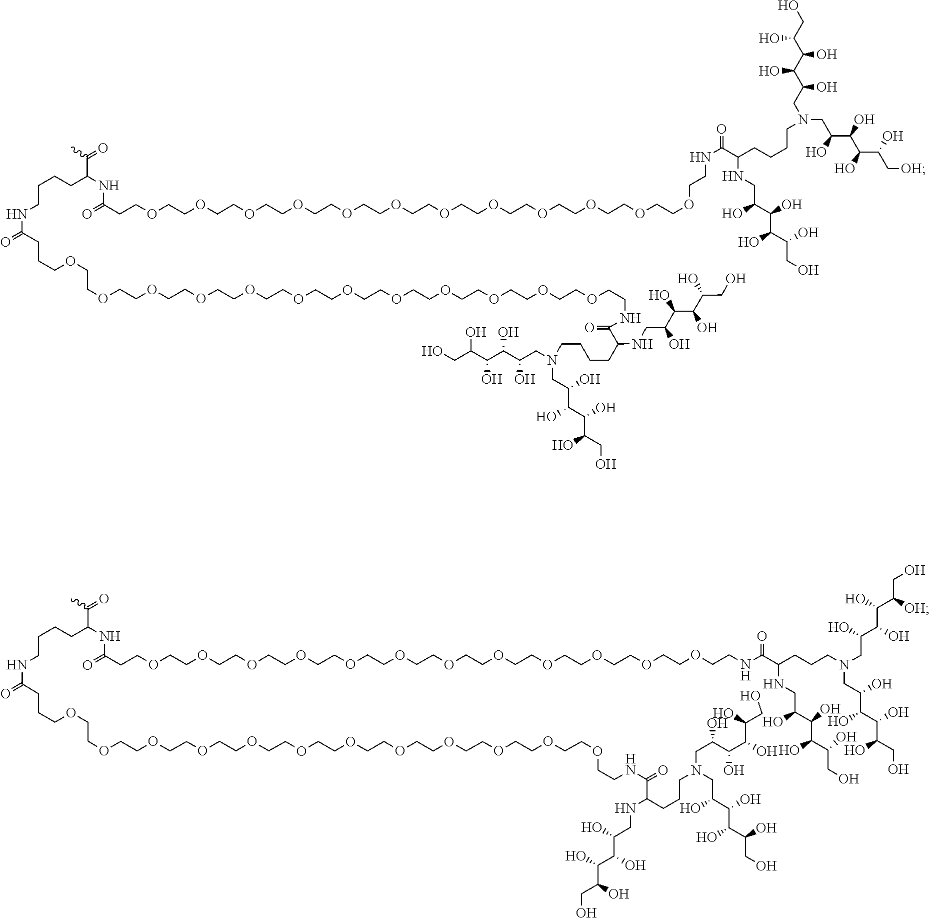

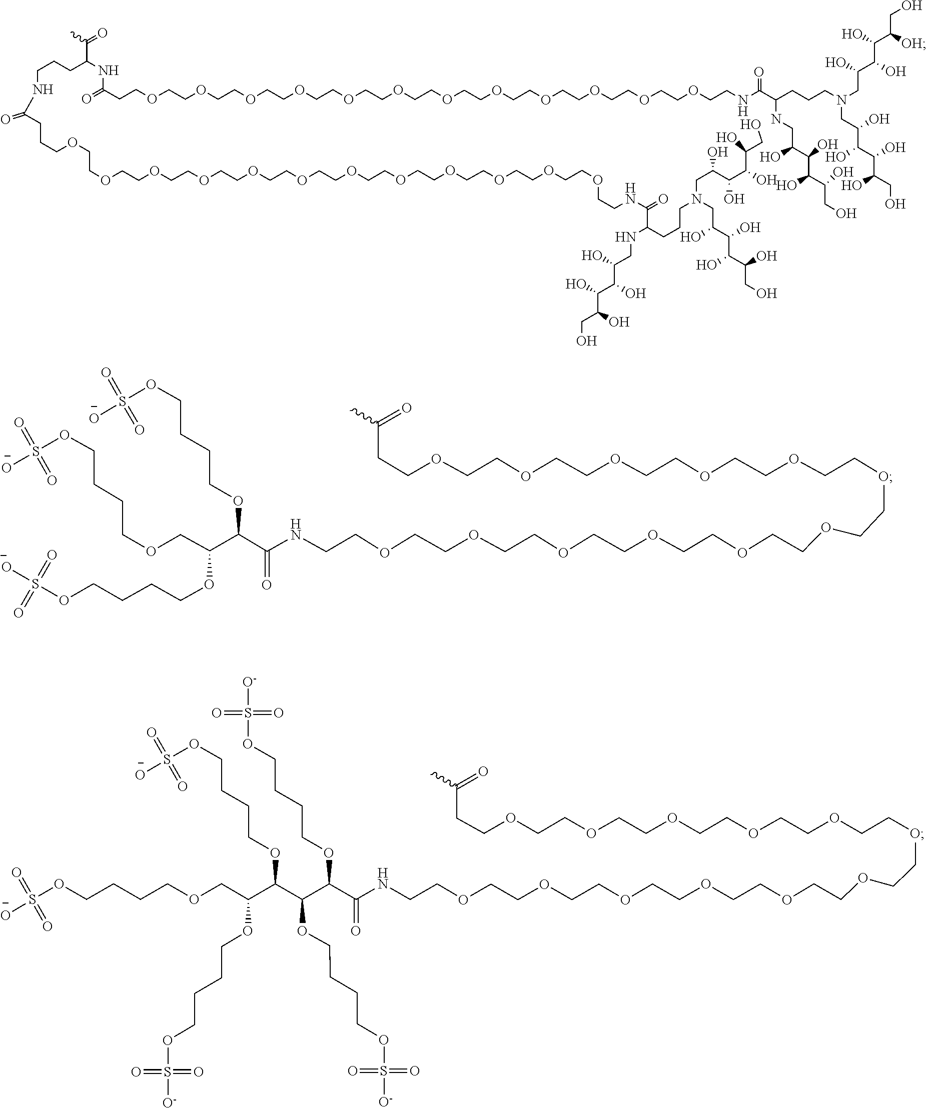

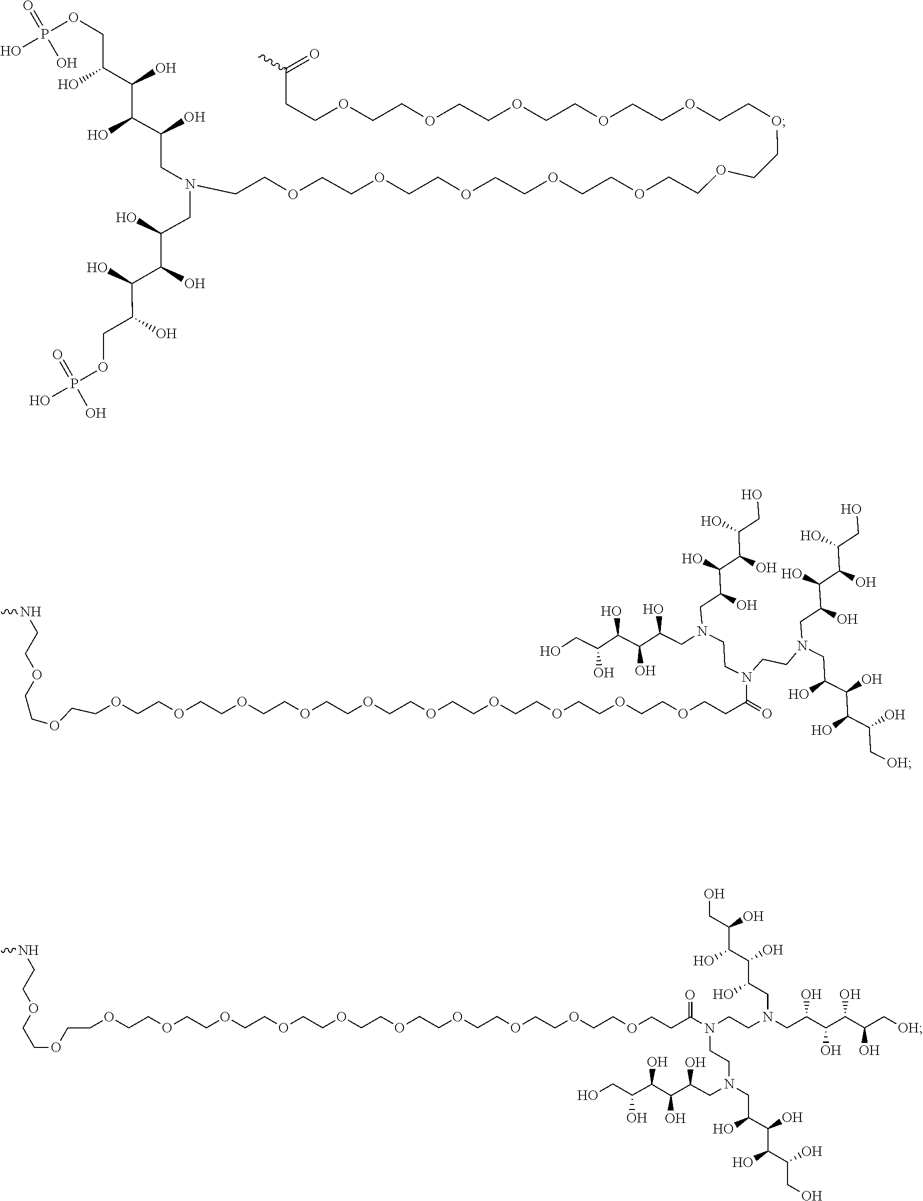

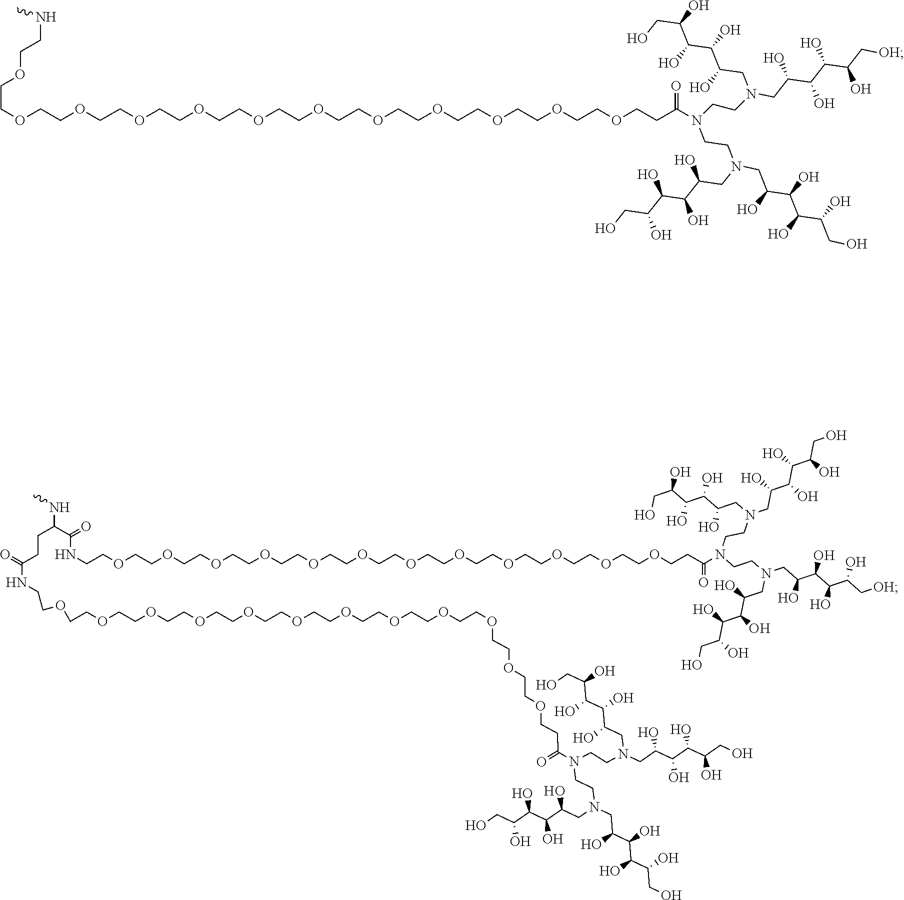

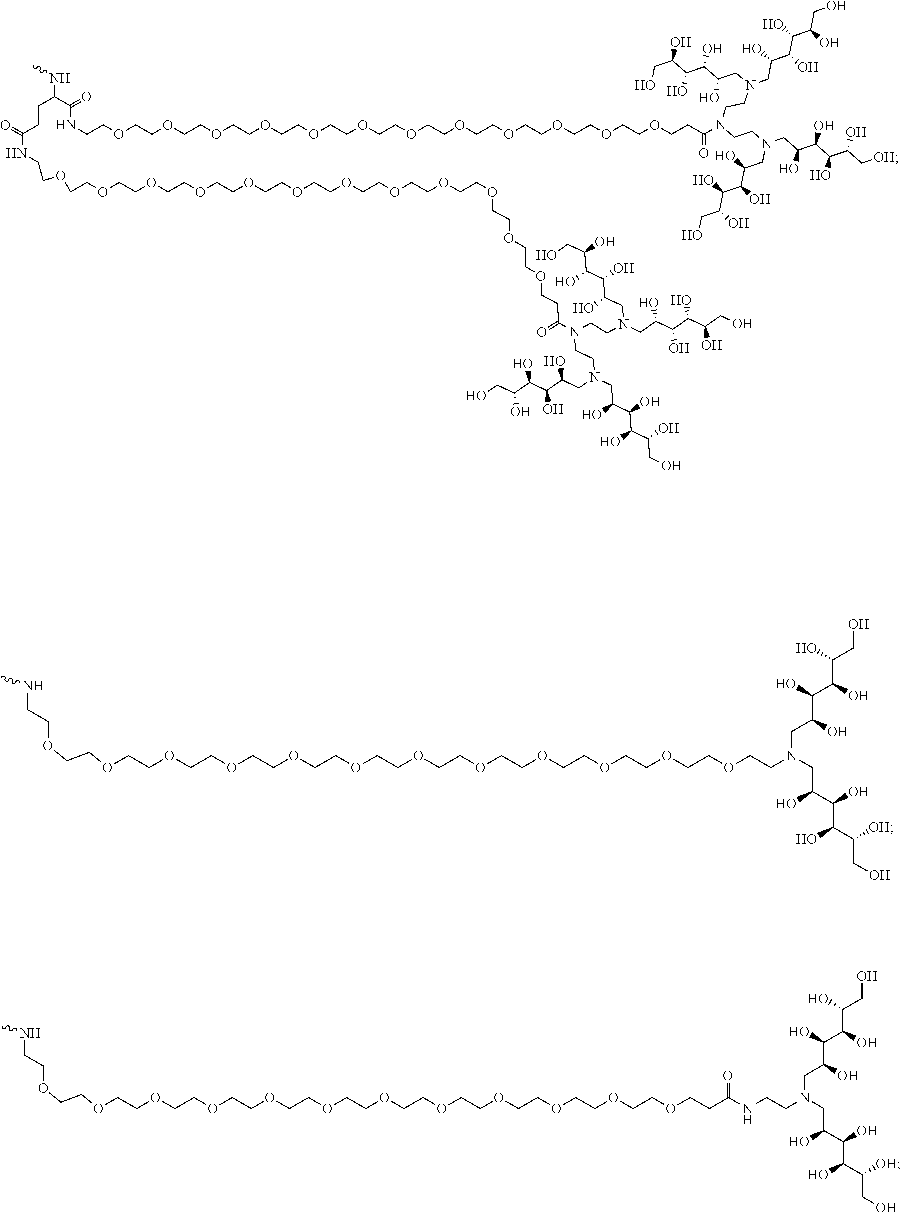

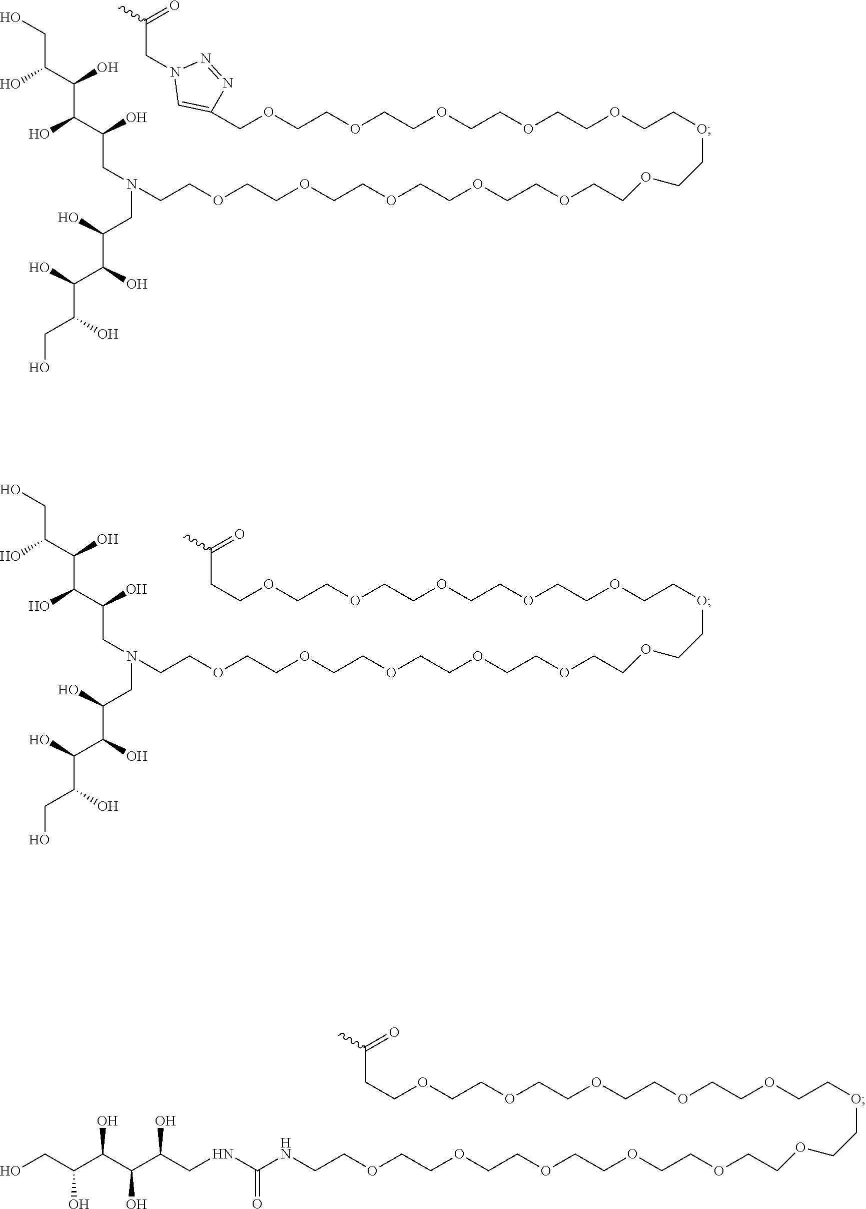

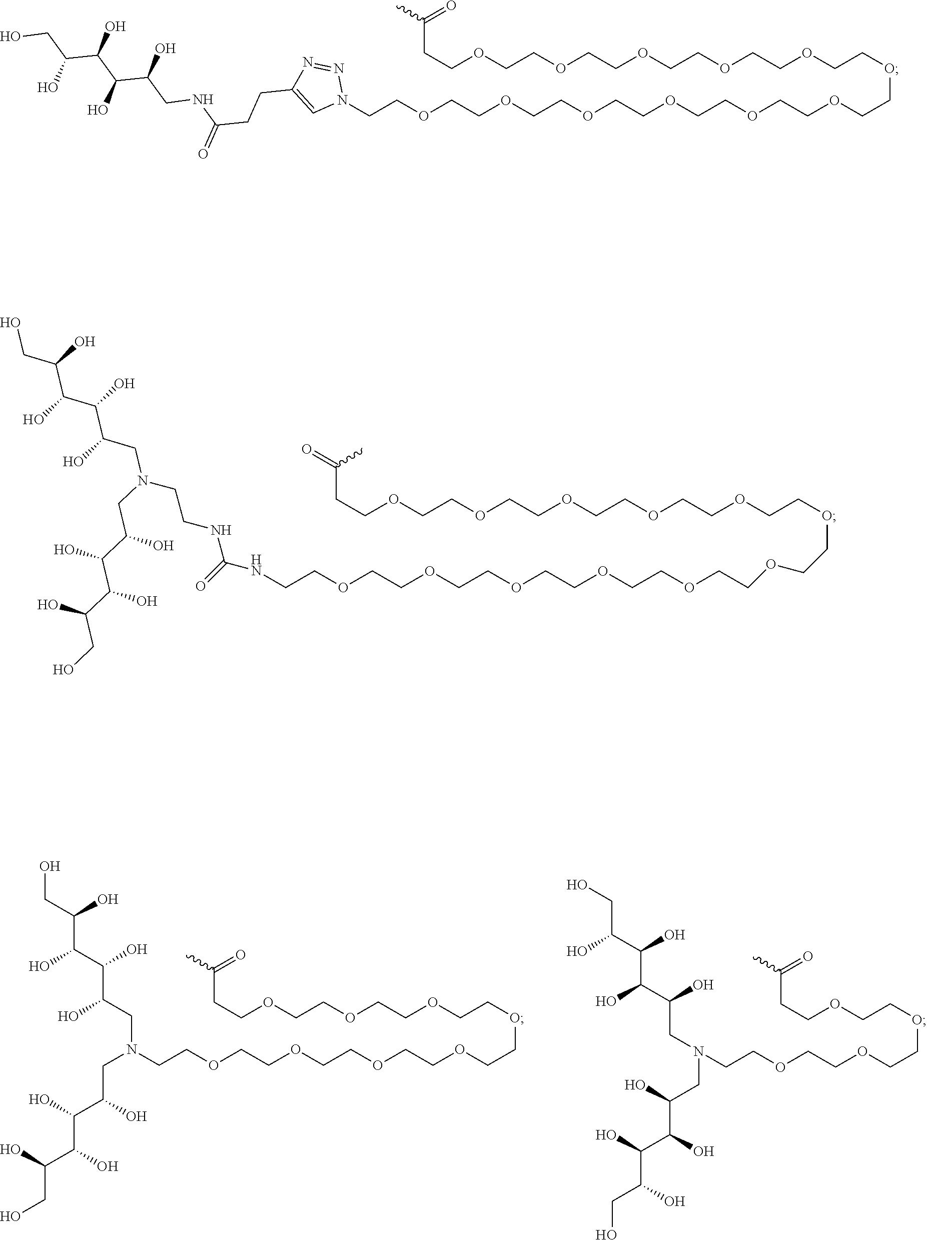

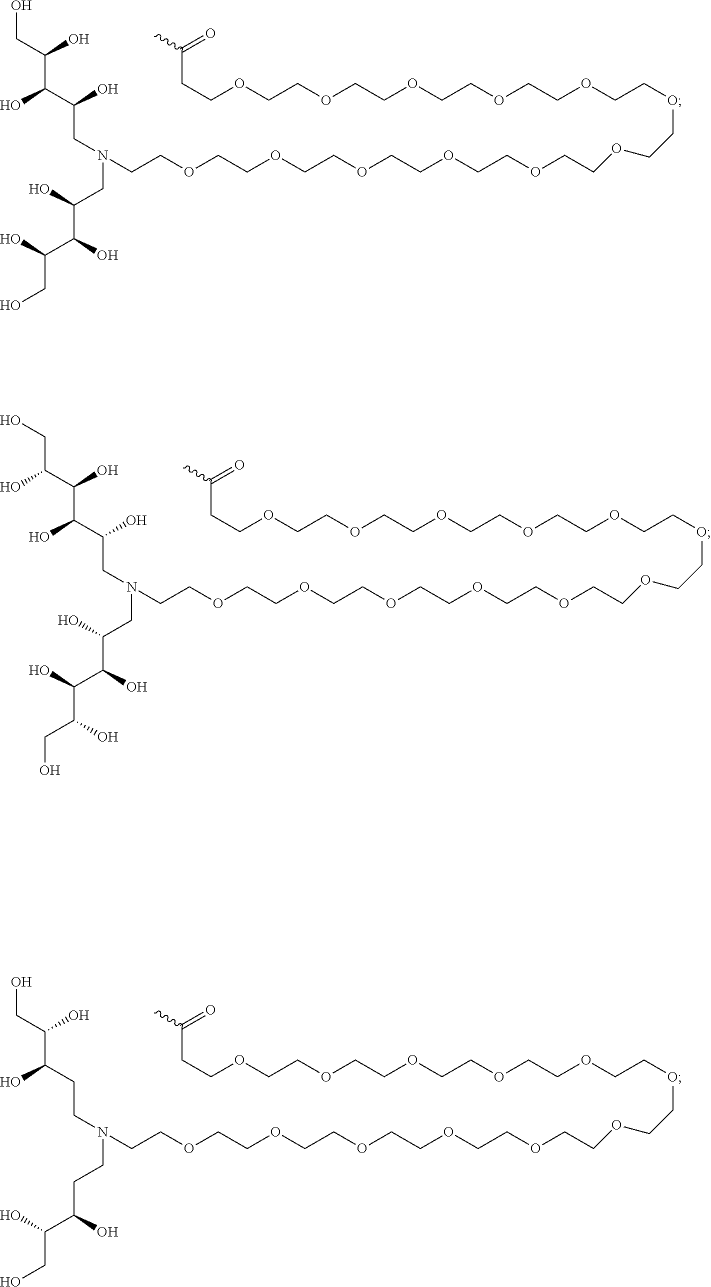

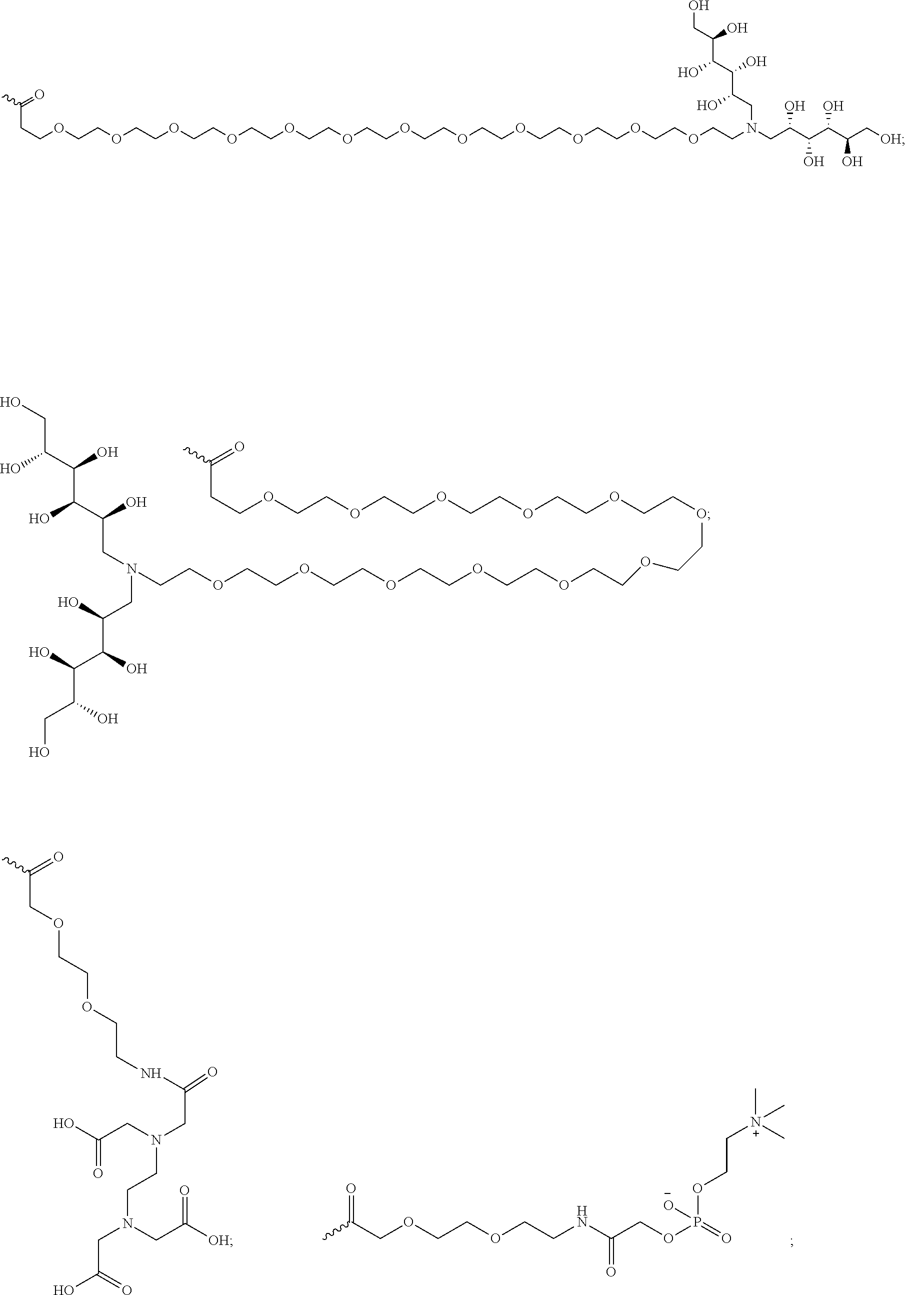

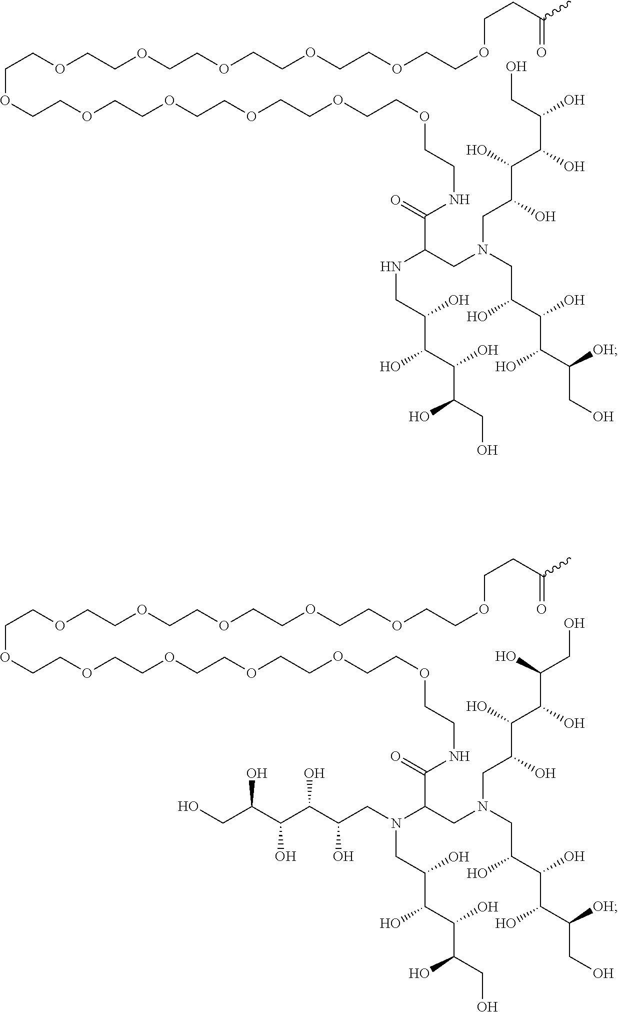

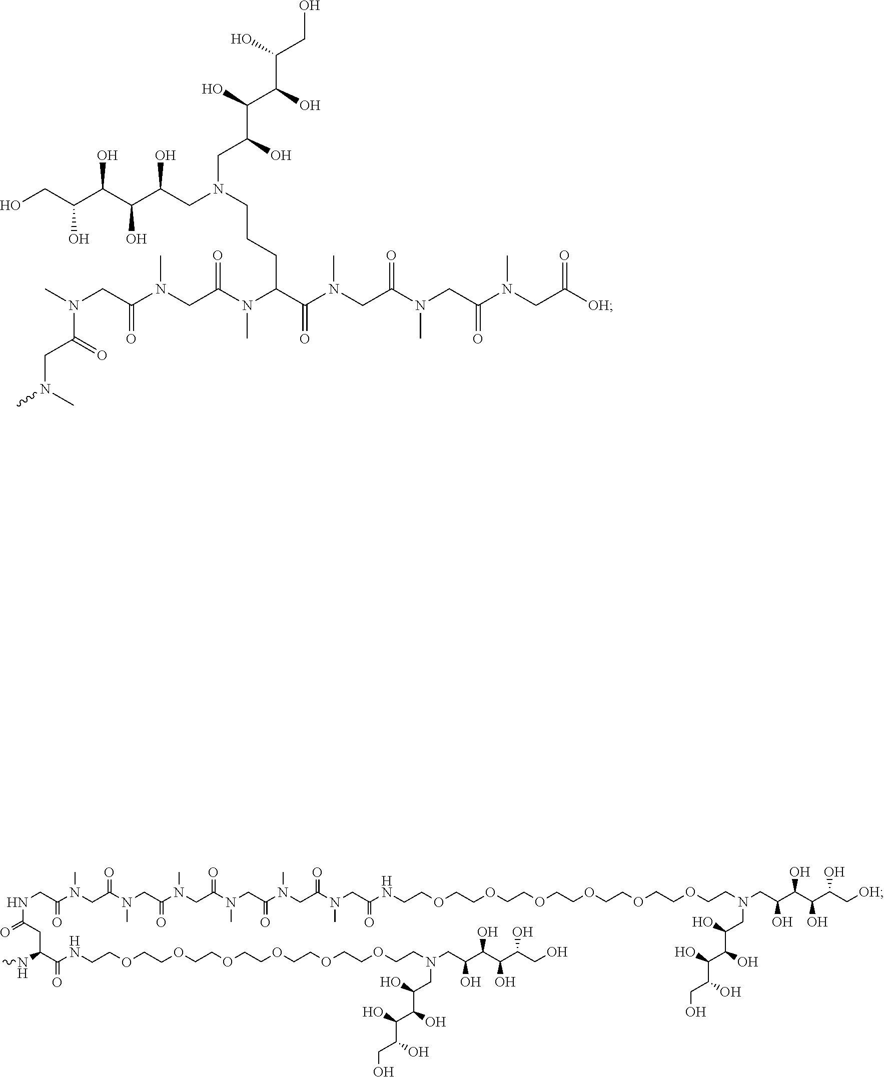

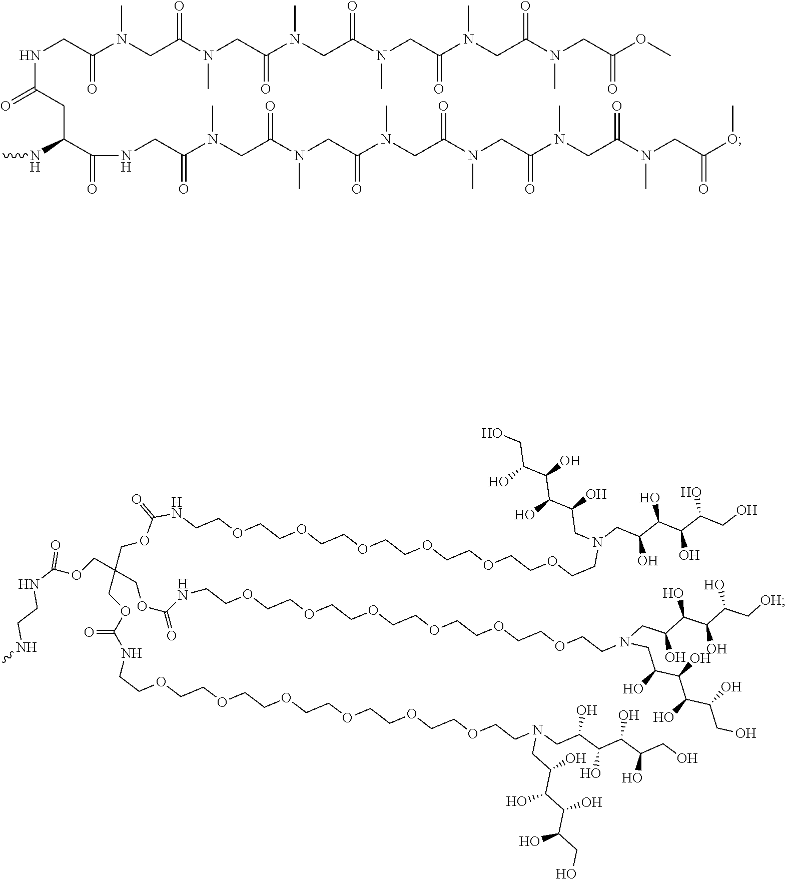

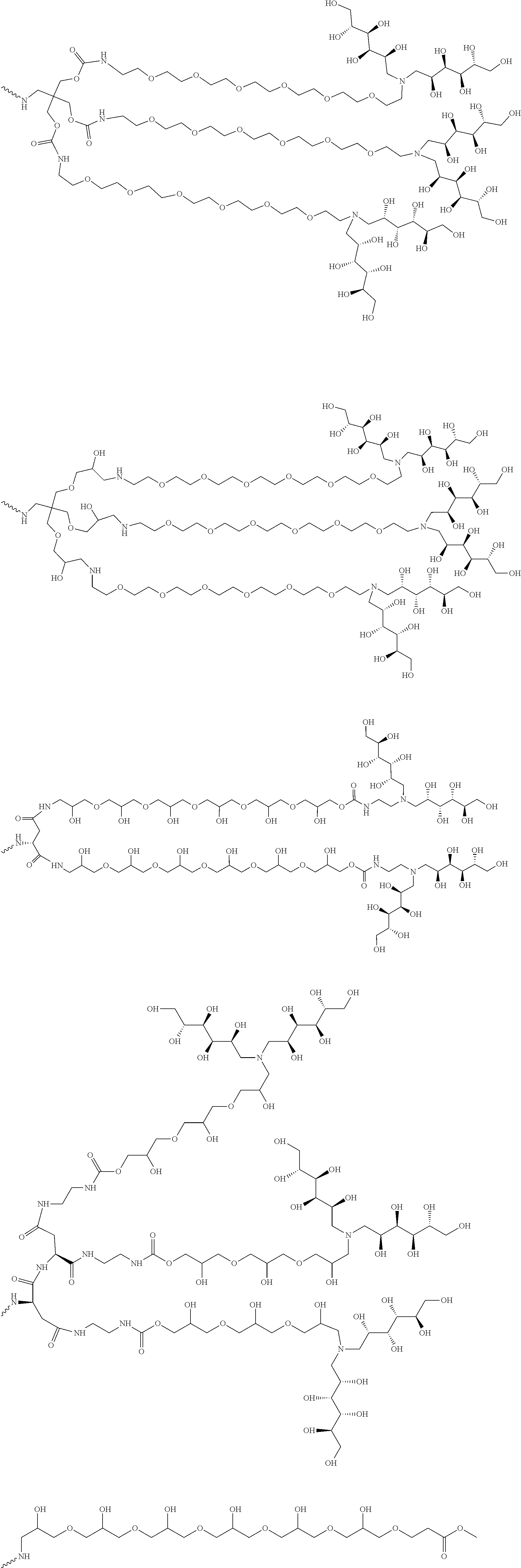

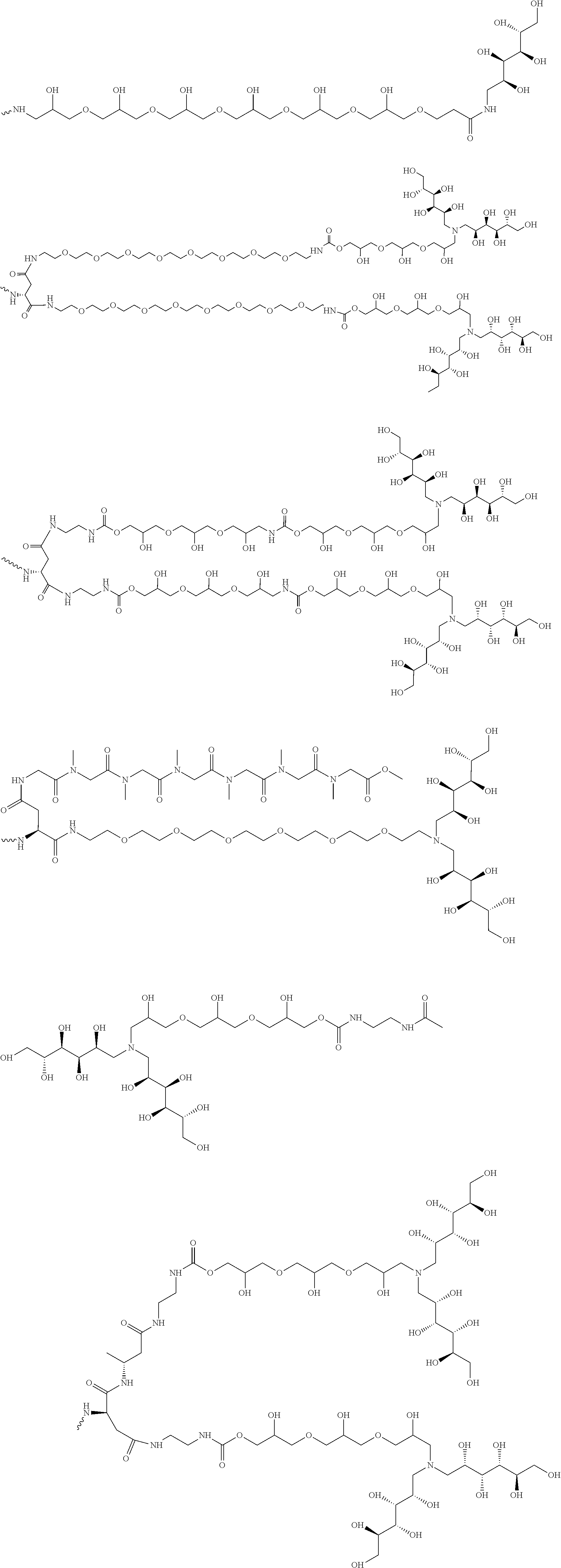

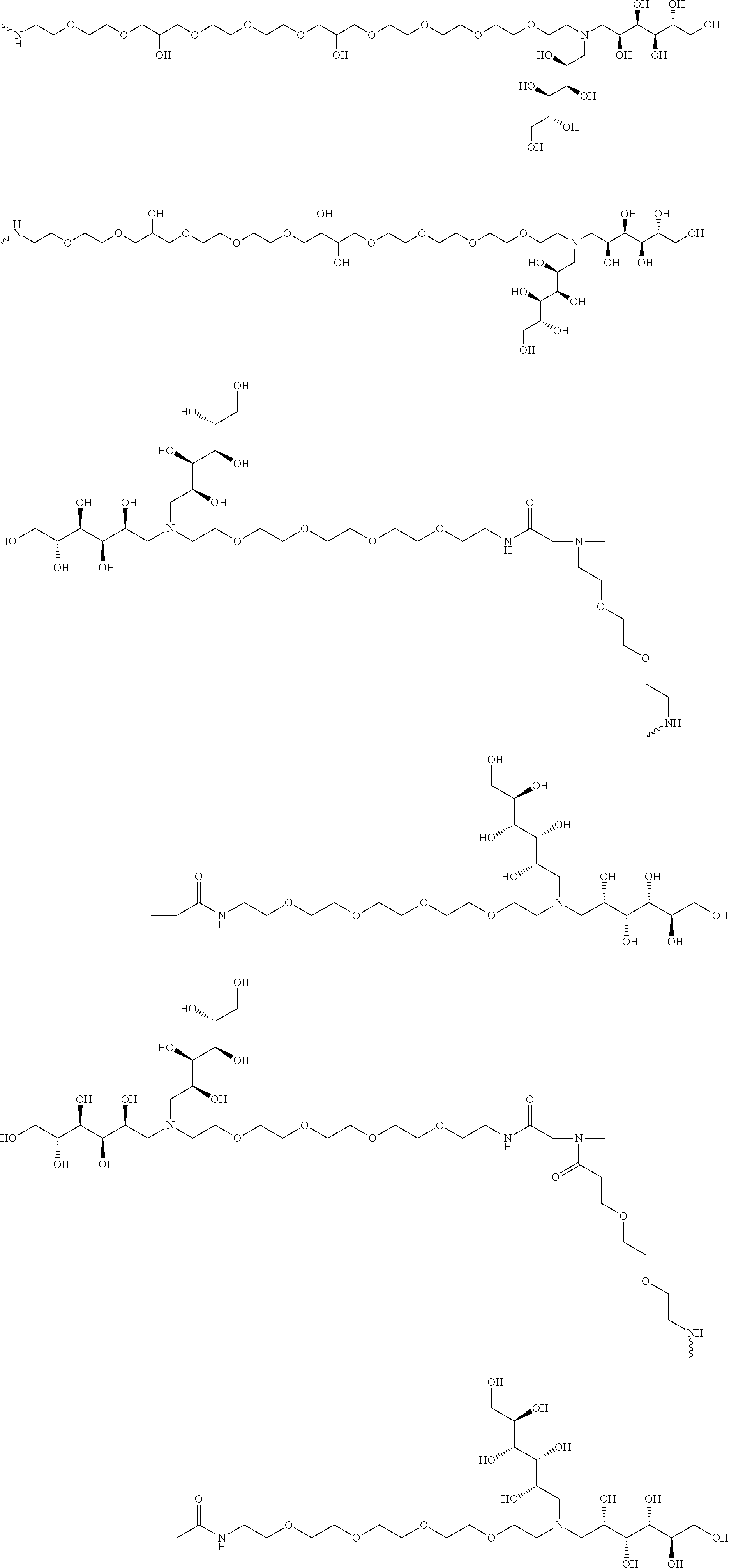

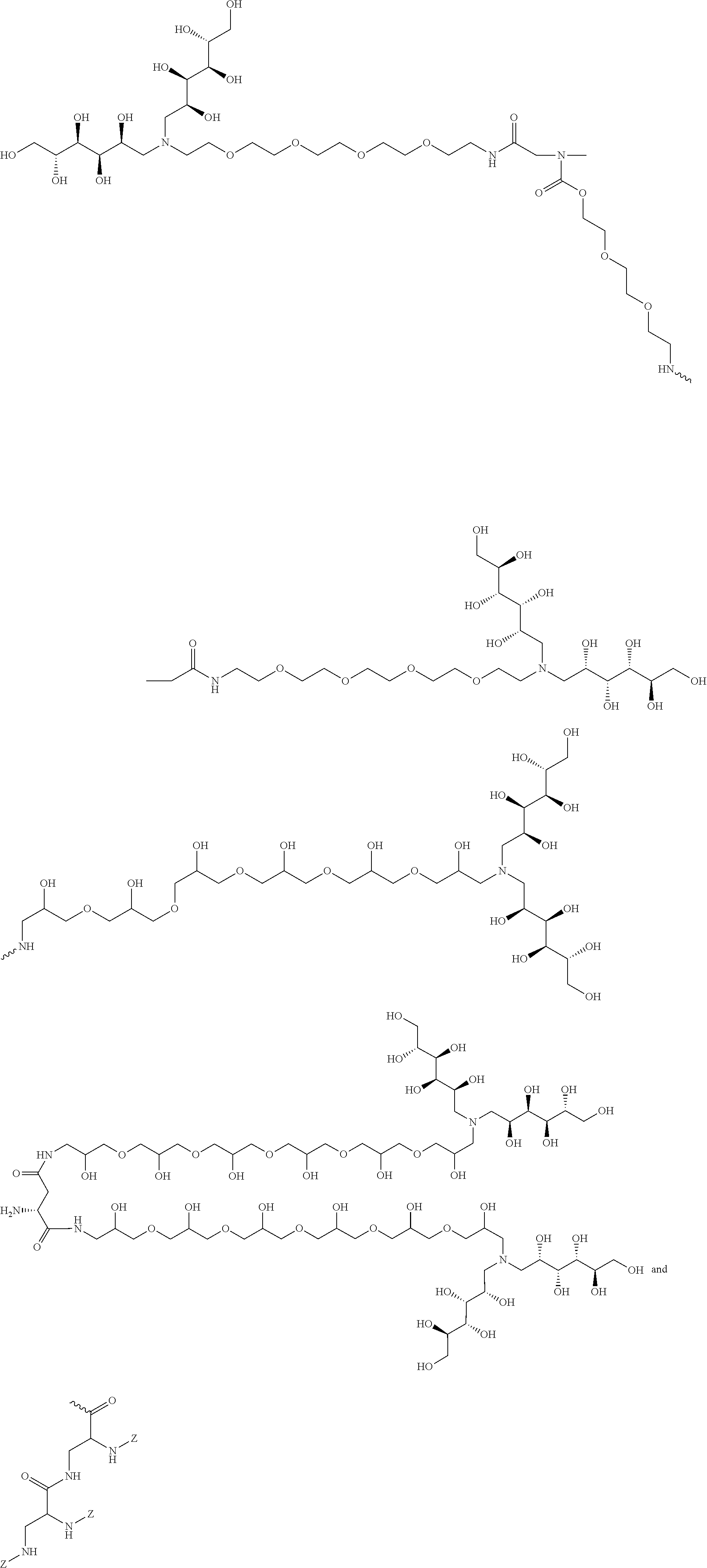

wherein each Z is attached at * and is individually selected from:

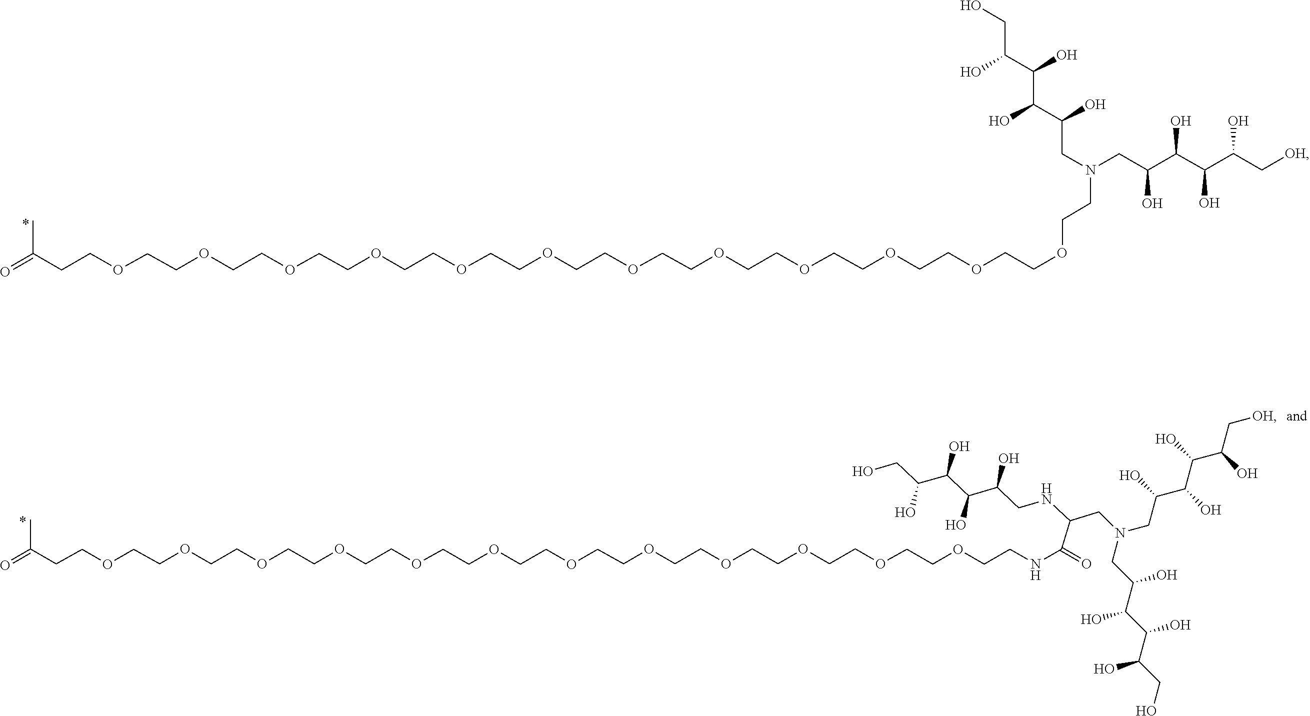

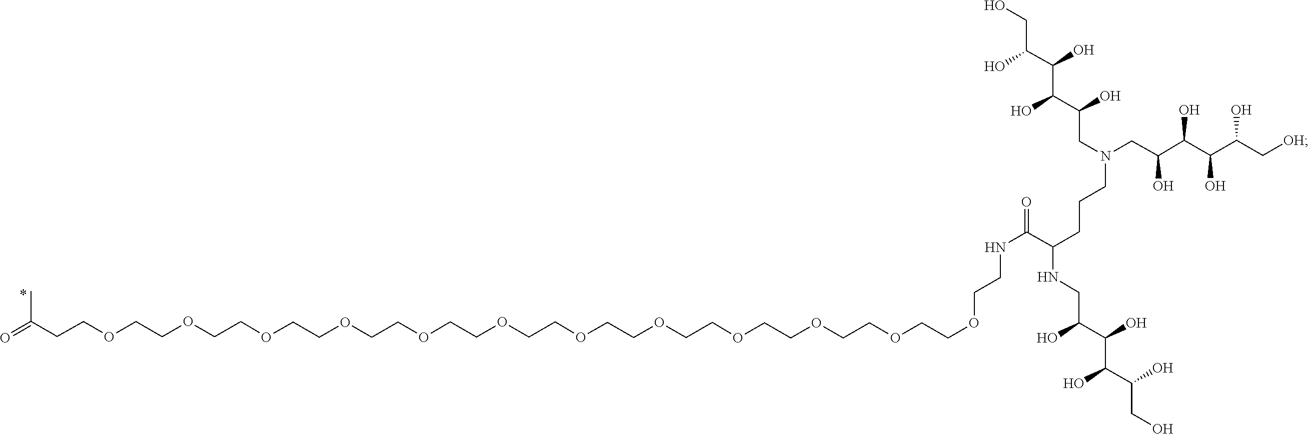

wherein each [symbol] is an attachment site to R^{b}, or to the enzyme-cleavable group.

Embodiment 62. The Linker compound of any one of Embodiments 1 to 61, comprising at least one Polar group comprising at least one Carboxyl unit having the following formula:

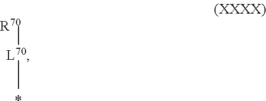

or a stereoisomer or salt thereof, wherein:

(a)
L⁷⁰ is selected from C₁-C₈ alkylene, C₁-C₈ alkylene-C(O)—, —C(O)—C₁-C₈ alkylene-, and —C(O)—C₁-C₈ alkylene-C(O)—, and * is an attachment site to R^{b}, to the enzyme-cleavable group, or to a remainder of the Polar group;
R⁷⁰ is —NR⁷¹(R⁷²-R⁷³), wherein R⁷¹ is selected from H, C₁-C₁₂ alkyl, substituted C₁-C₁₂ alkyl, or polyethylene glycol (optionally having 1 to 12 ethylene glycol subunits), R⁷² is a bond or is selected from optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl, and R⁷³ is a carboxyl or polycarboxyl, wherein polycarboxyl comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide; or

(b)
L⁷⁰ is selected from C₁-C₈ alkylene, C₁-C₈ alkylene-C(O)—, —C(O)—C₁-C₈ alkylene-, and —C(O)—C₁-C₈ alkylene-C(O)—, and * is an attachment site to R^{b}, to the enzyme-cleavable group, or to a remainder of the Polar group;
R⁷⁰ is —NR⁷¹(R⁷⁵—(R⁷³)₂), wherein R⁷¹ is selected from H, C₁-C₁₂ alkyl, substituted C₁-C₁₂ alkyl, or polyethylene glycol (optionally having 1 to 12 ethylene glycol subunits), R⁷⁵ is a branched optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl and each R⁷³ is independently carboxyl or polycarboxyl, wherein polycarboxyl comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide; or

(c)
L⁷⁰ is selected from C₁-C₈ alkylene, C₁-C₈ alkylene-C(O)—, —C(O)—C₁-C₈ alkylene-, and —C(O)—C₁-C₈ alkylene-C(O)—, and * is an attachment site to R^{b}, to the enzyme-cleavable group, or to a remainder of the Polar group;
R⁷⁰ is —N(R⁷⁴-R⁷³)(R⁷²-R⁷³), wherein R⁷² and R⁷⁴ are each independently selected from optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl, and each R⁷³ is independently carboxyl or polycarboxyl, wherein the polycarboxyl comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide.

Embodiment 63. The Linker compound of any one of Embodiments 1 to 62, comprising a Polar group including the Polymer unit and a Sugar unit.

Embodiment 64. The Linker compound of any one of Embodiments 1 to 62, comprising a Polar group including at least two Polymer units.

Embodiment 65. The Linker compound of any one of Embodiments 1 to 62, comprising a Polar group including the Polymer unit and a Carboxyl unit.

Embodiment 66. The Linker compound of any one of Embodiments 1 to 62, comprising at least two Polar groups.

Embodiment 67. The Linker compound of any one of Embodiments 1 to 62, comprising a Polar group including the Polymer unit, the Sugar unit and the Carboxyl unit.

Embodiment 68. The Linker compound of any one of Embodiments 1 to 62, comprising a Polar group including at least two Polymer units, at least one Sugar unit and at least one Carboxyl unit.

Embodiment 69. The Linker compound of any one of Embodiments 1 to 62, wherein the enzyme-cleavable group comprises at least two amino acids.

Embodiment 70. The Linker compound of any one of Embodiments 1 to 69, comprising at least one of the Polar group attached to the enzyme-cleavable group.

Embodiment 71. The Linker compound of any one of Embodiments 1-70, having one of the following structures:

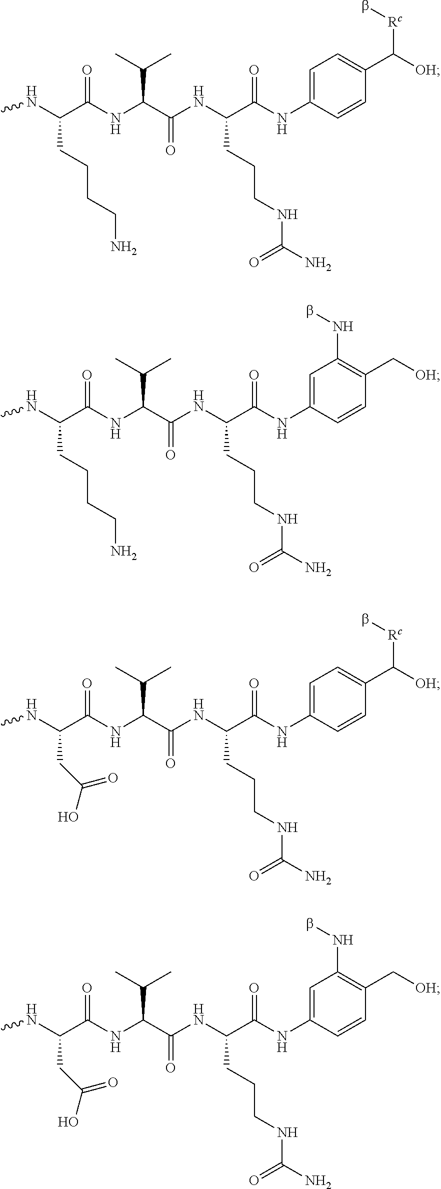

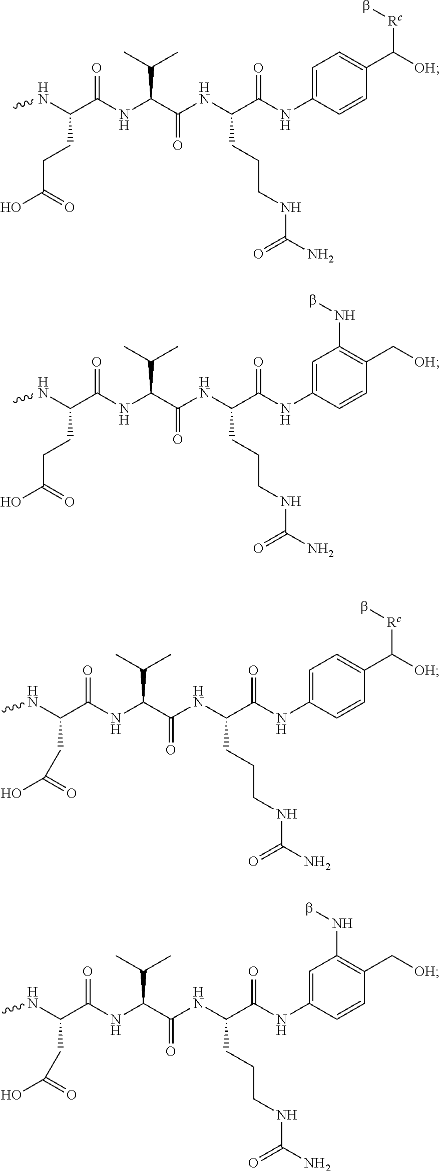

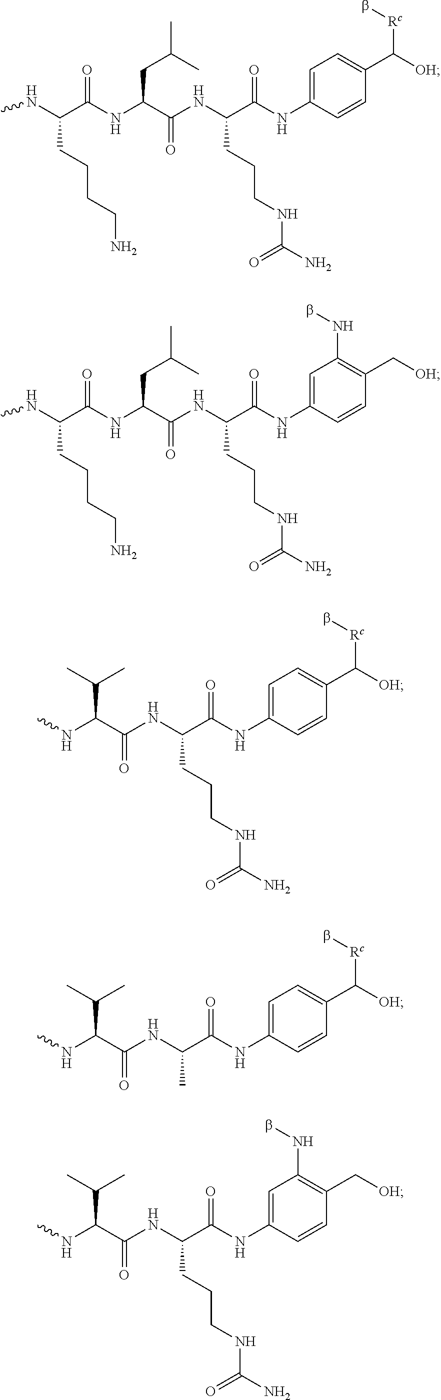

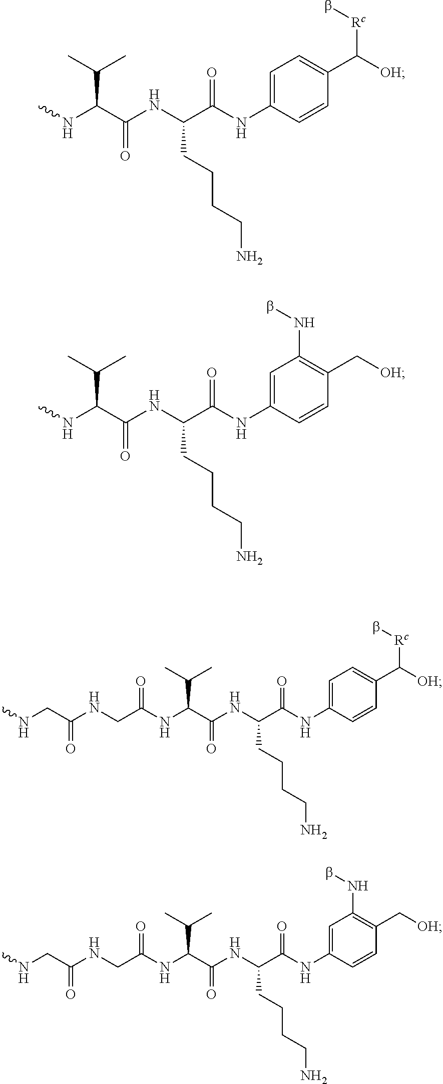

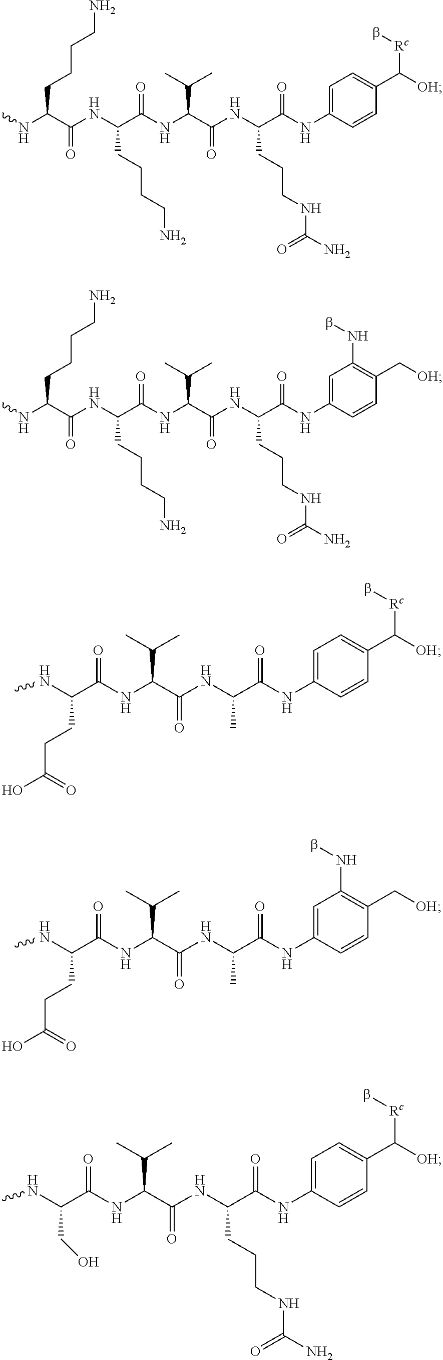

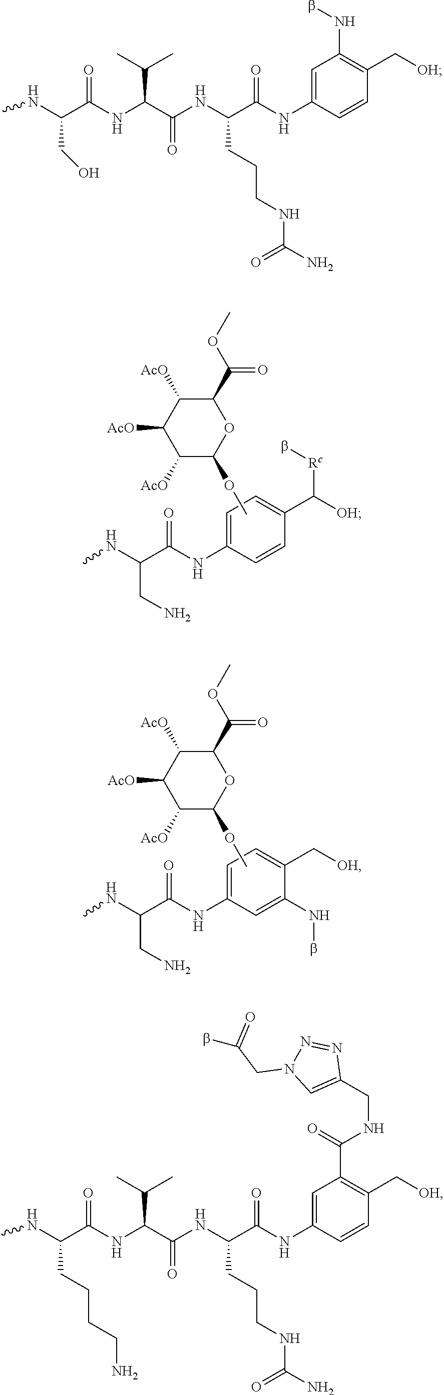

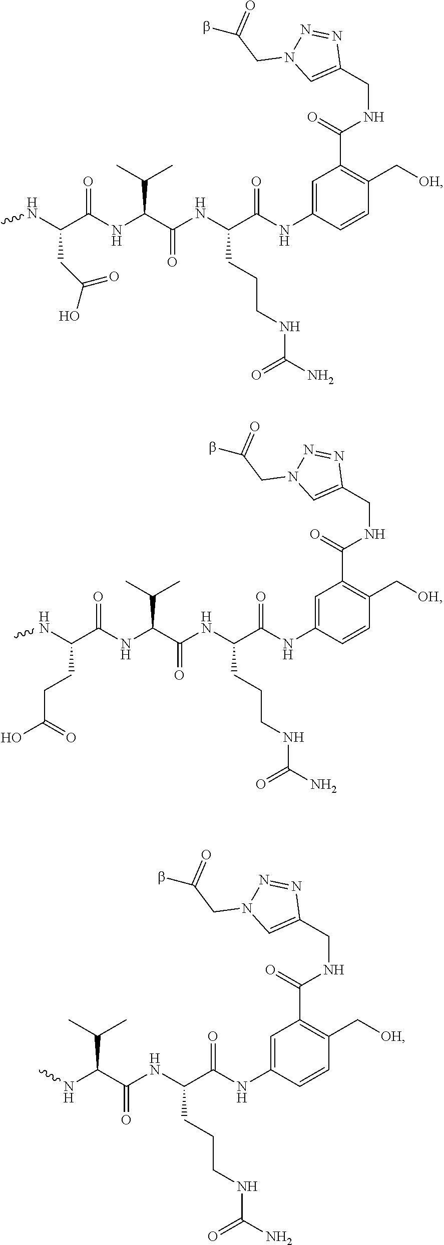

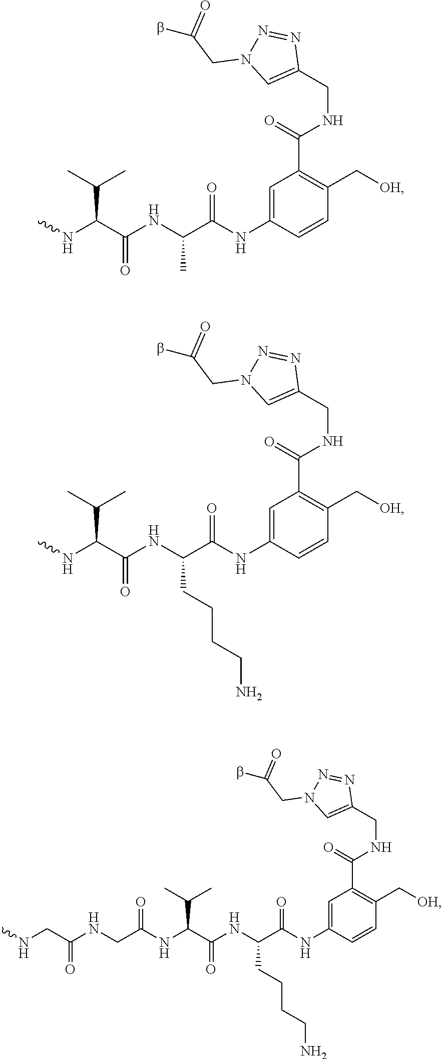

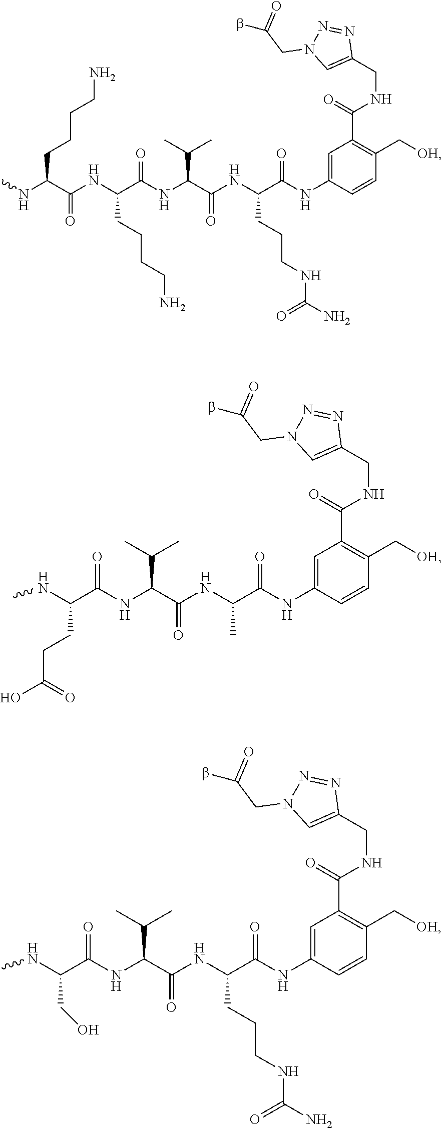

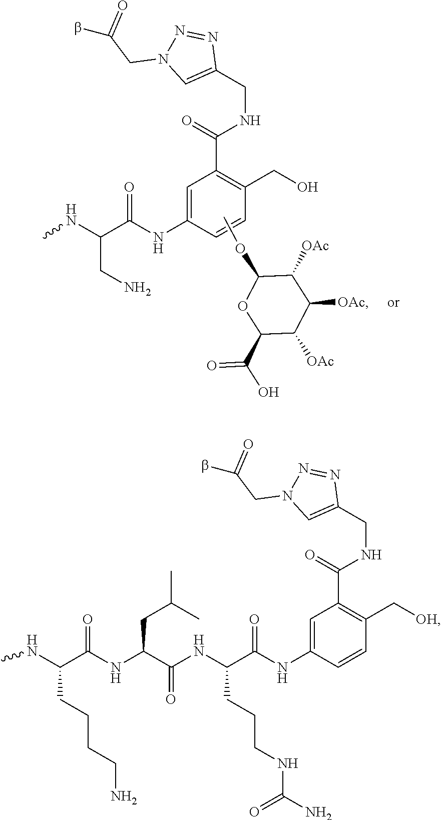

wherein
R^{c} is a bond or C₁₋₆ alkylene;
the wavy line on the amino group indicates an attachment site for a Stretcher group or, prior to attachment to the Stretcher group, indicates H;
β—is the attachment site to the at least one Polar group; and
the benzylic H on the benzylic OH is optionally replaced with a bond to at least one of the Drug unit or to the linking group attached to at least one of the Drug units.

Embodiment 72. The Linker compound of any one of Embodiments 1-71, wherein the enzyme-cleavable group comprises a peptide that is cleavable by an intracellular protease.

Embodiment 73. The Linker compound of any one of Embodiments 1-71, wherein the enzyme-cleavable group comprises a glucuronide-cleavable moiety.

Embodiment 74. The Linker compound of Embodiment 73, wherein the enzyme-cleavable group comprises a cleavable peptide including a valine-citrulline peptide, a valine-alanine peptide, a valine-lysine peptide, a phenylalanine-lysine peptide, or a glycine-glycine-phenylalanine-glycine peptide.

Embodiment 75. The Linker compound of any one of Embodiments 1 to 74, wherein the Linker unit comprises a para-aminobenzyl alcohol self immolative group (PABA).

Embodiment 76. The Linker compound of any one of Embodiments 1-75, comprising one of the following structures:

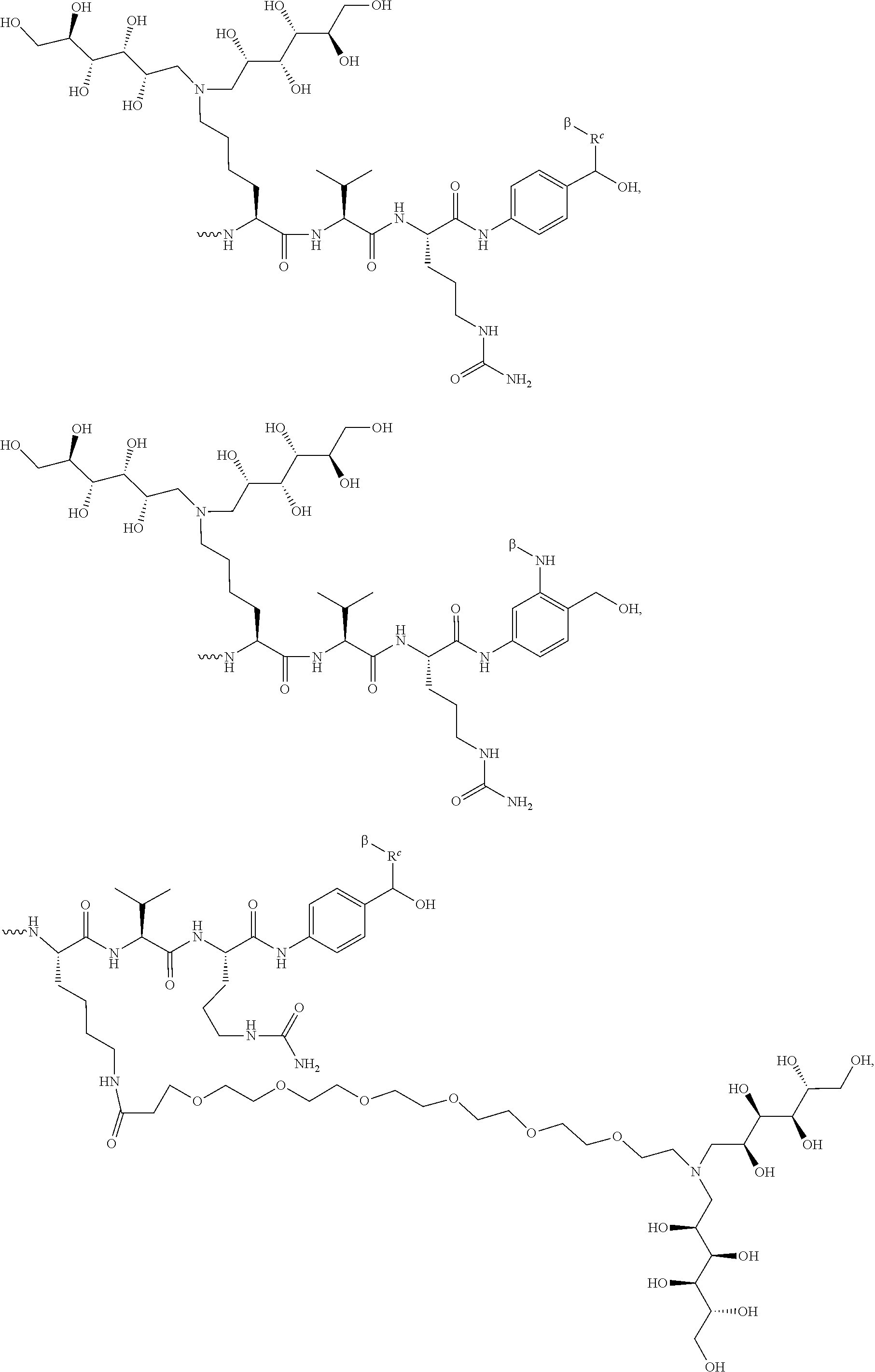

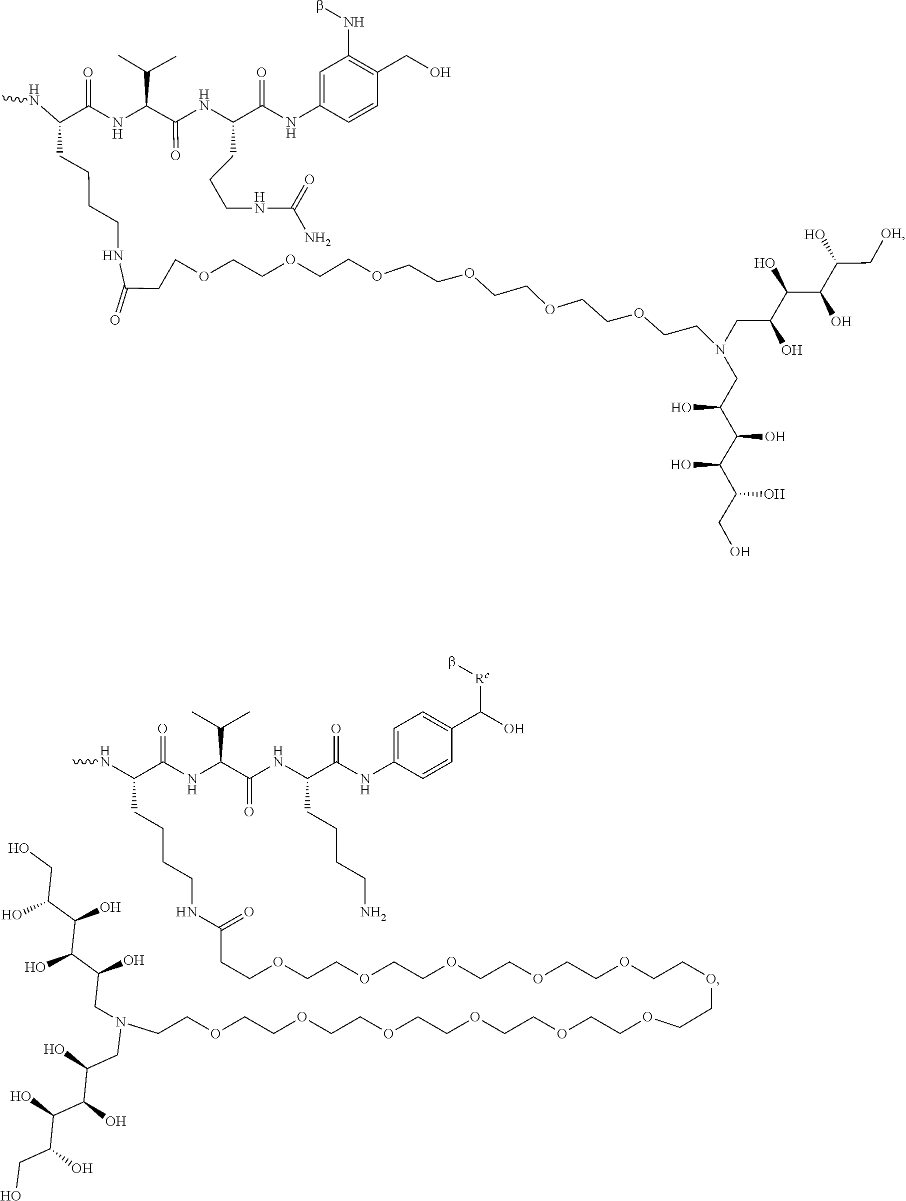

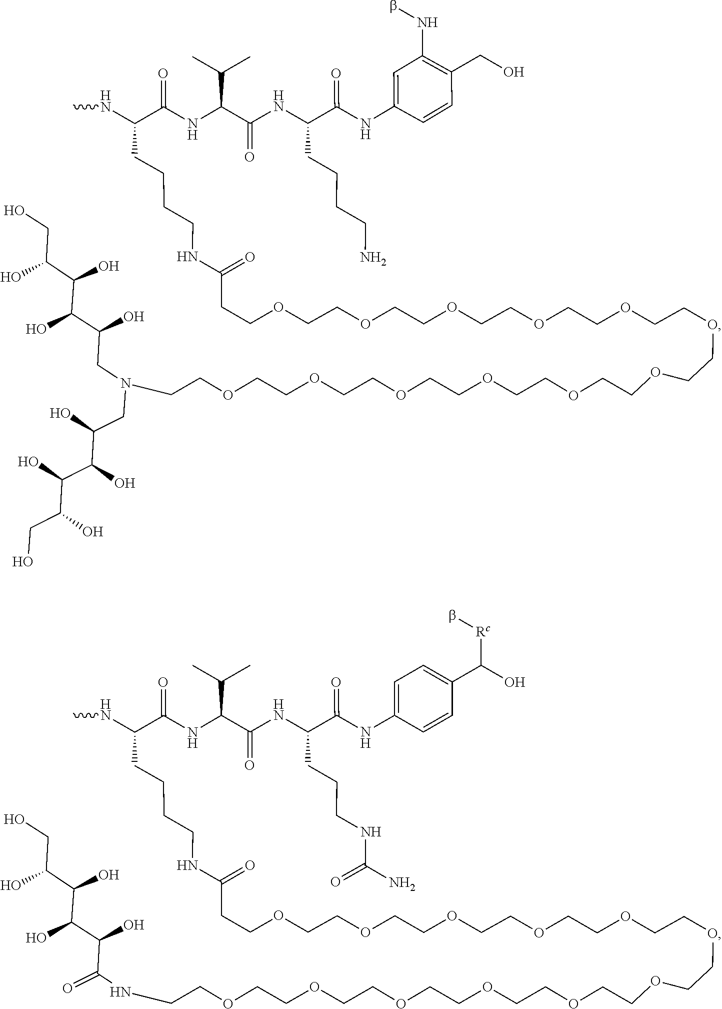

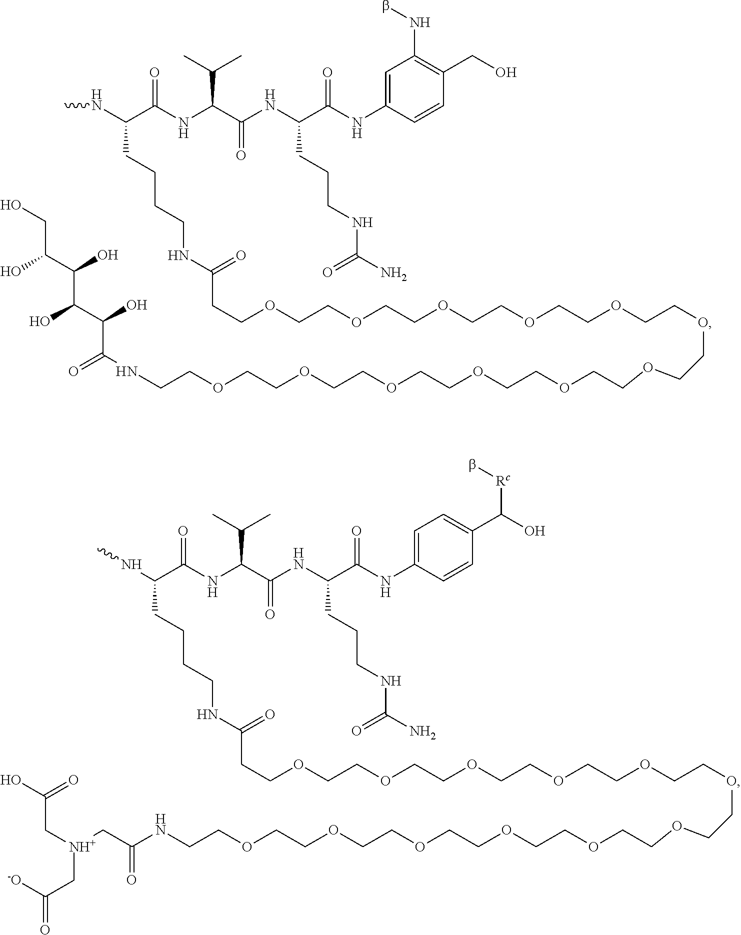

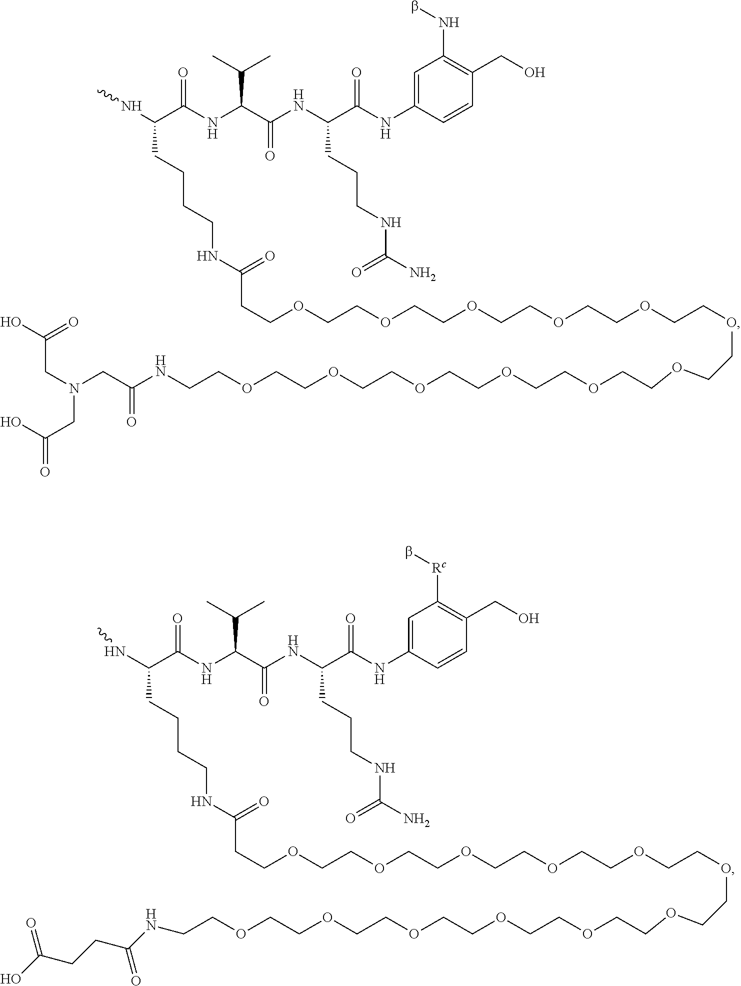

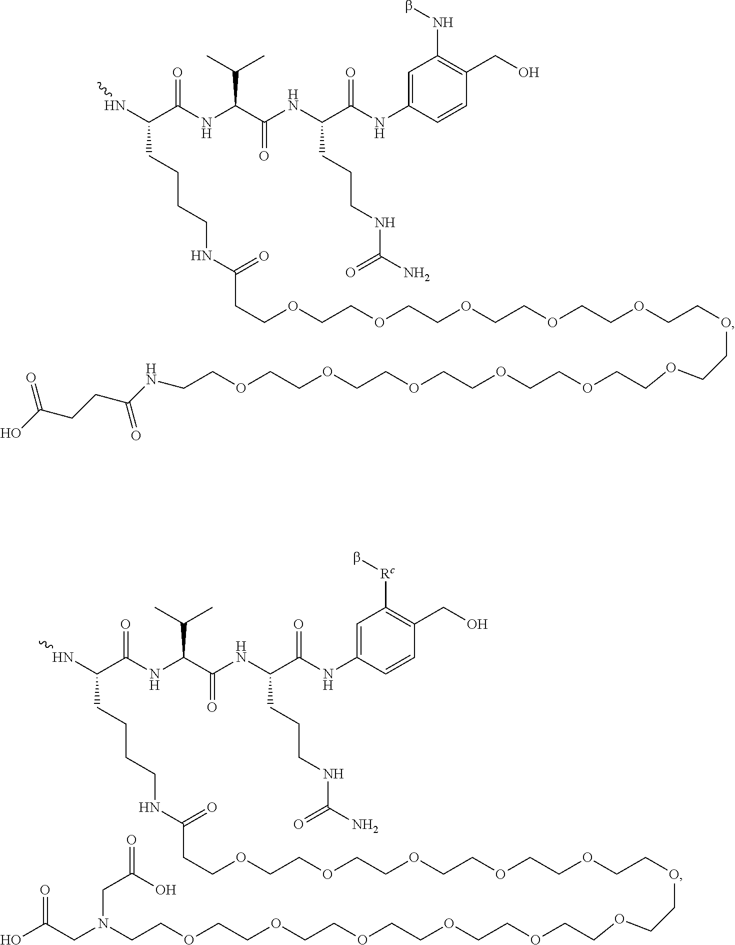

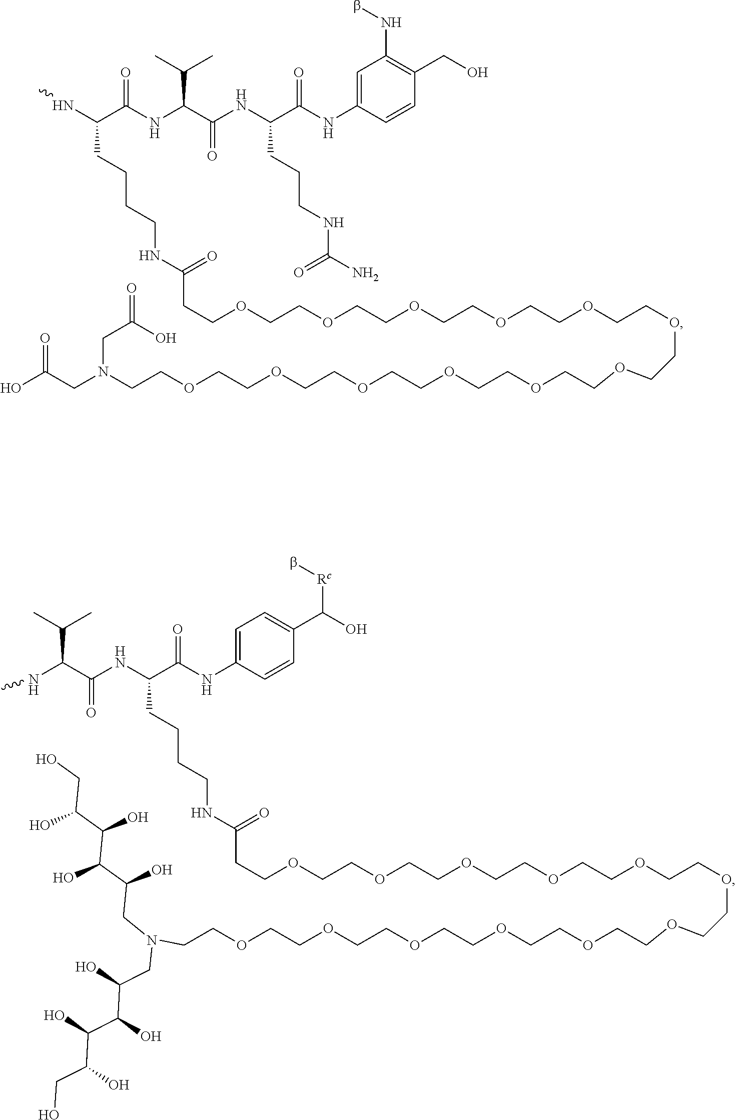

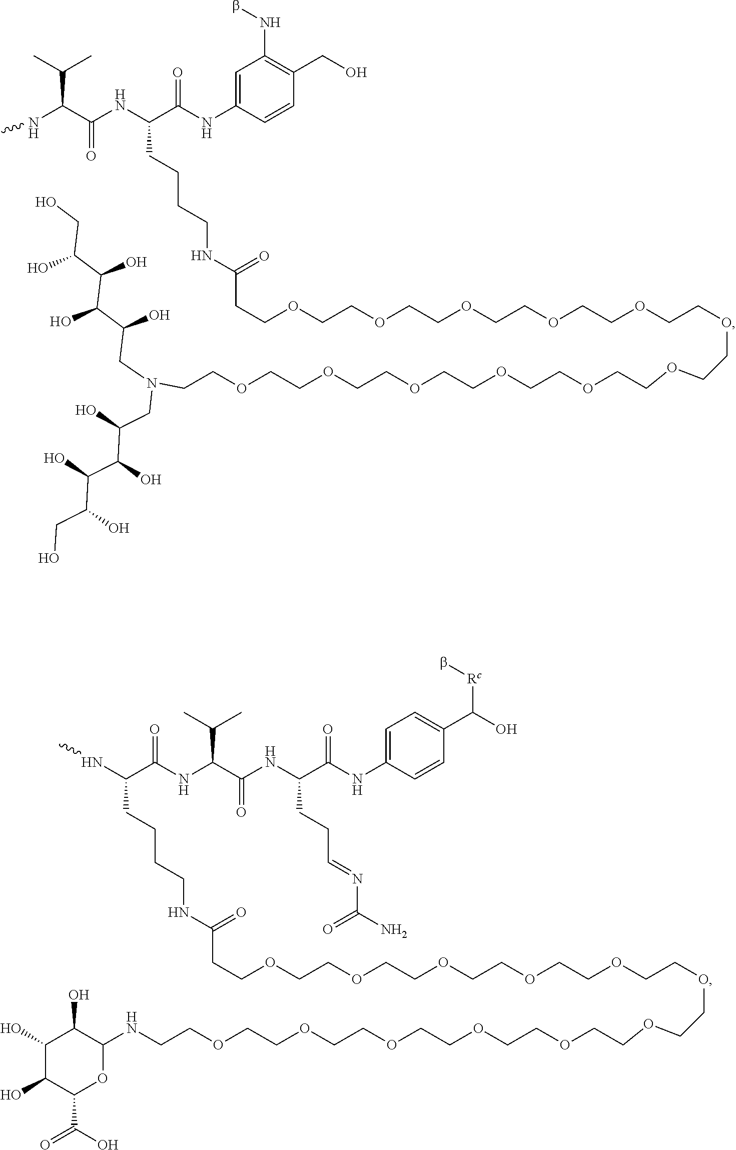

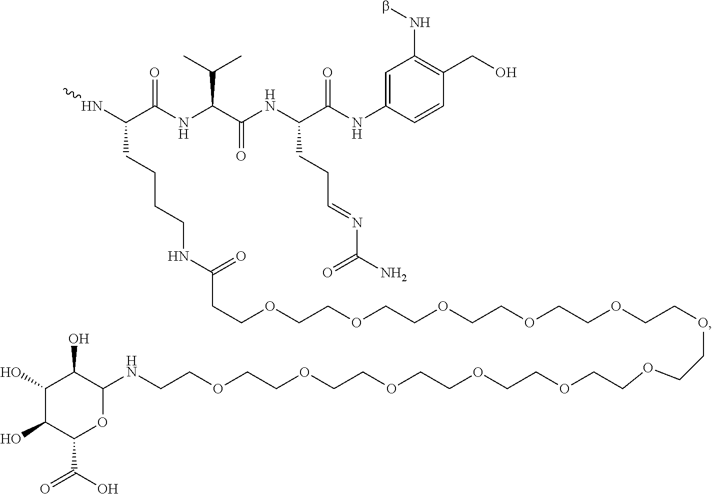

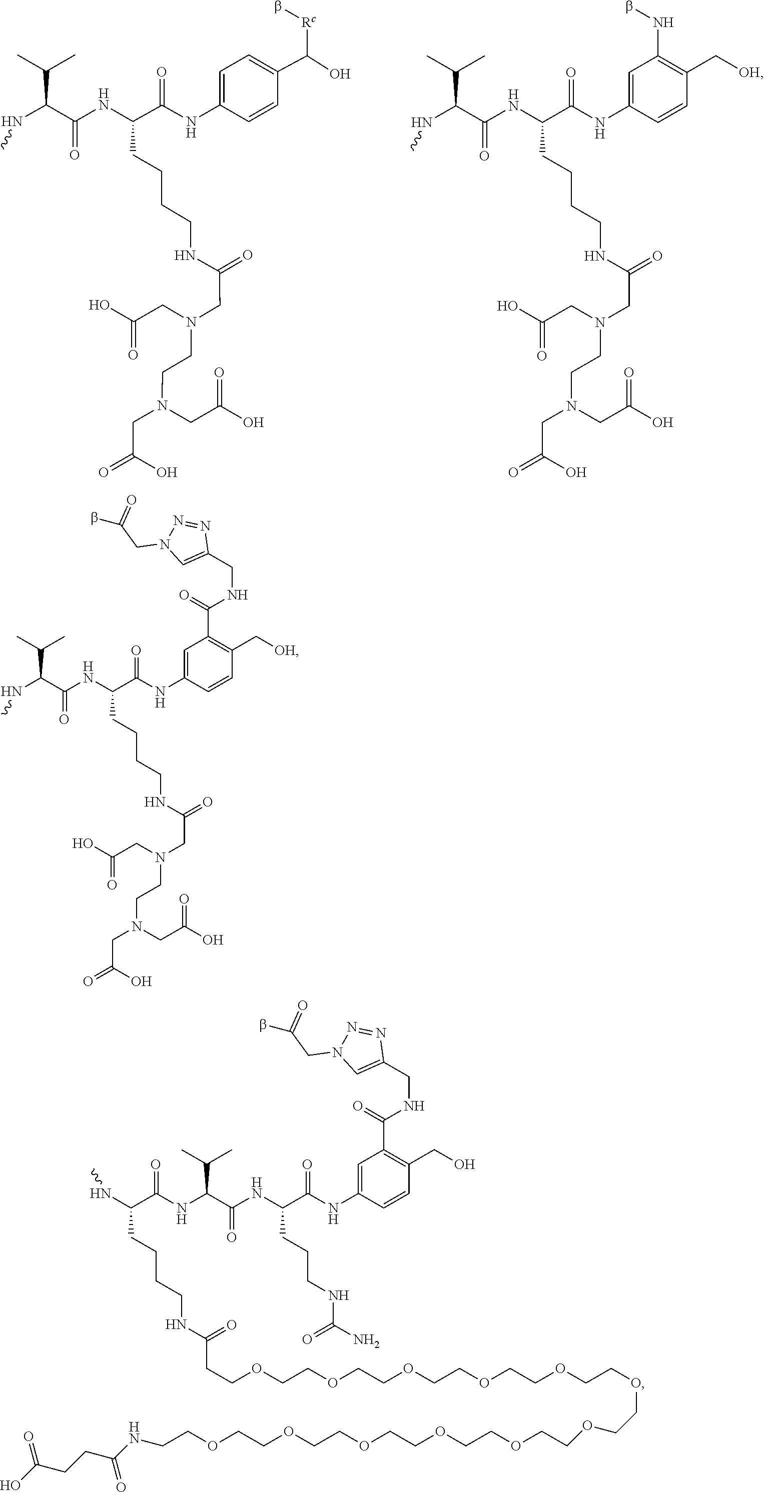

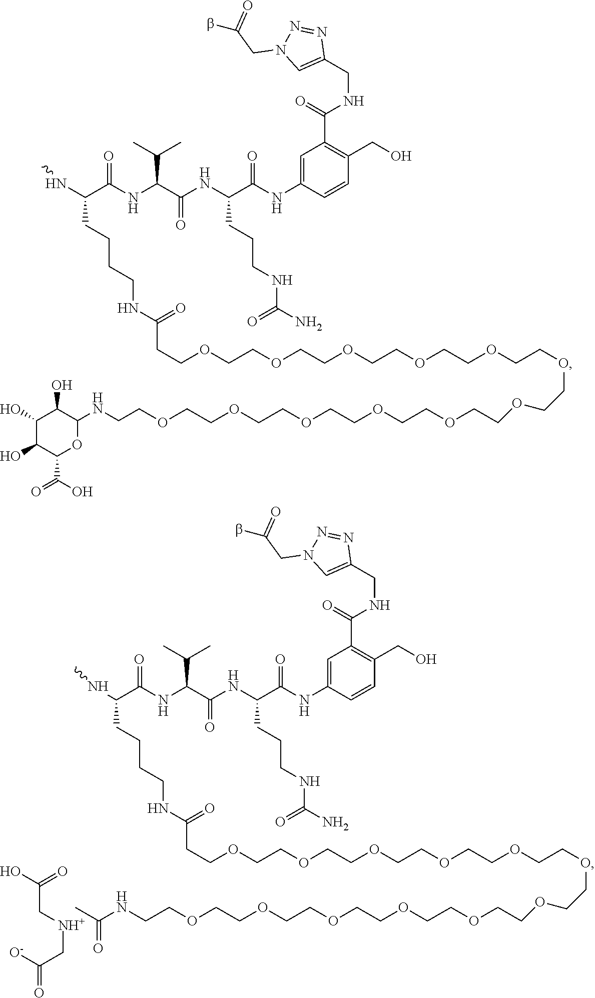

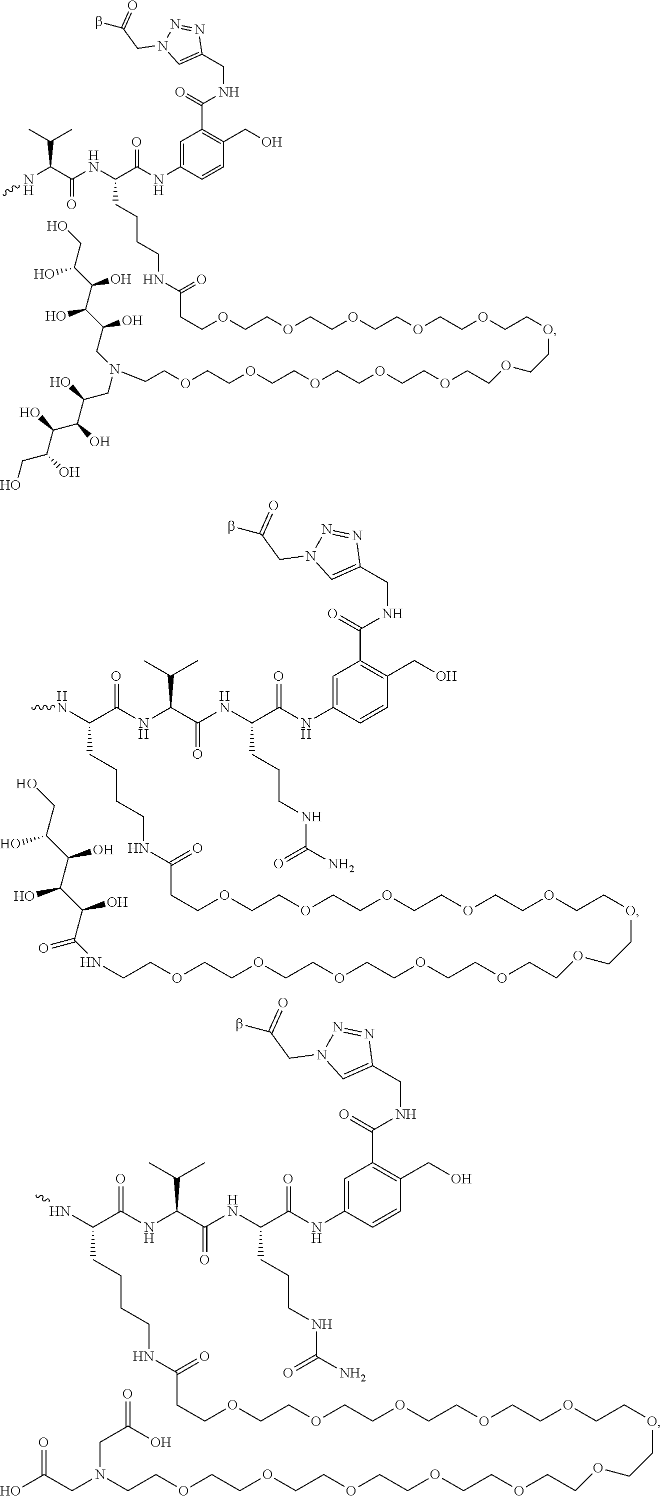

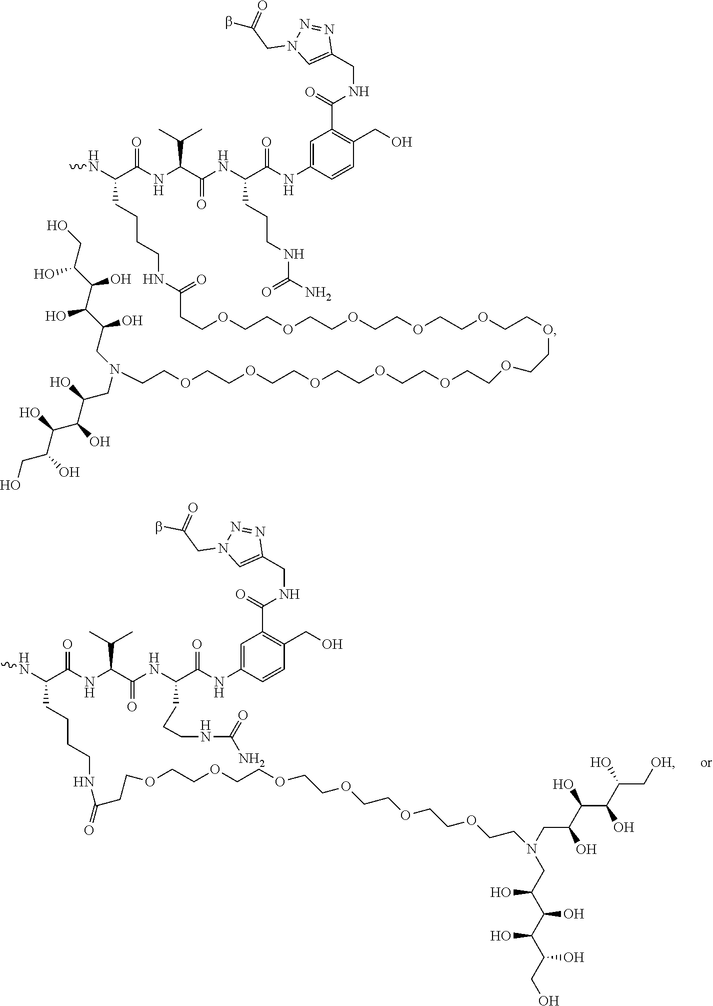

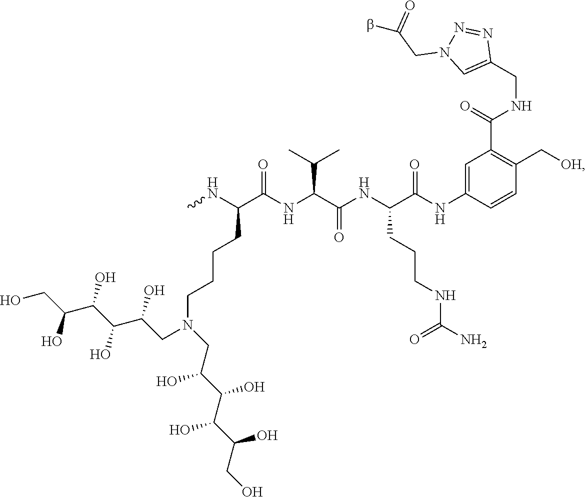

wherein
R^{c} is a bond or C₁₋₆ alkylene;
the wavy line on the amino group indicates an attachment site for the Stretcher group or, prior to attachment to the Stretcher group, indicates H;
β—is an attachment site to a POLY unit; and
the H on the benzylic OH is optionally replaced with a bond to at least one of the Drug units or to the attachment site to at least one of the Drug units.

Embodiment 77. The Linker compound of any one of Embodiments 1-5, having one of the following structures:

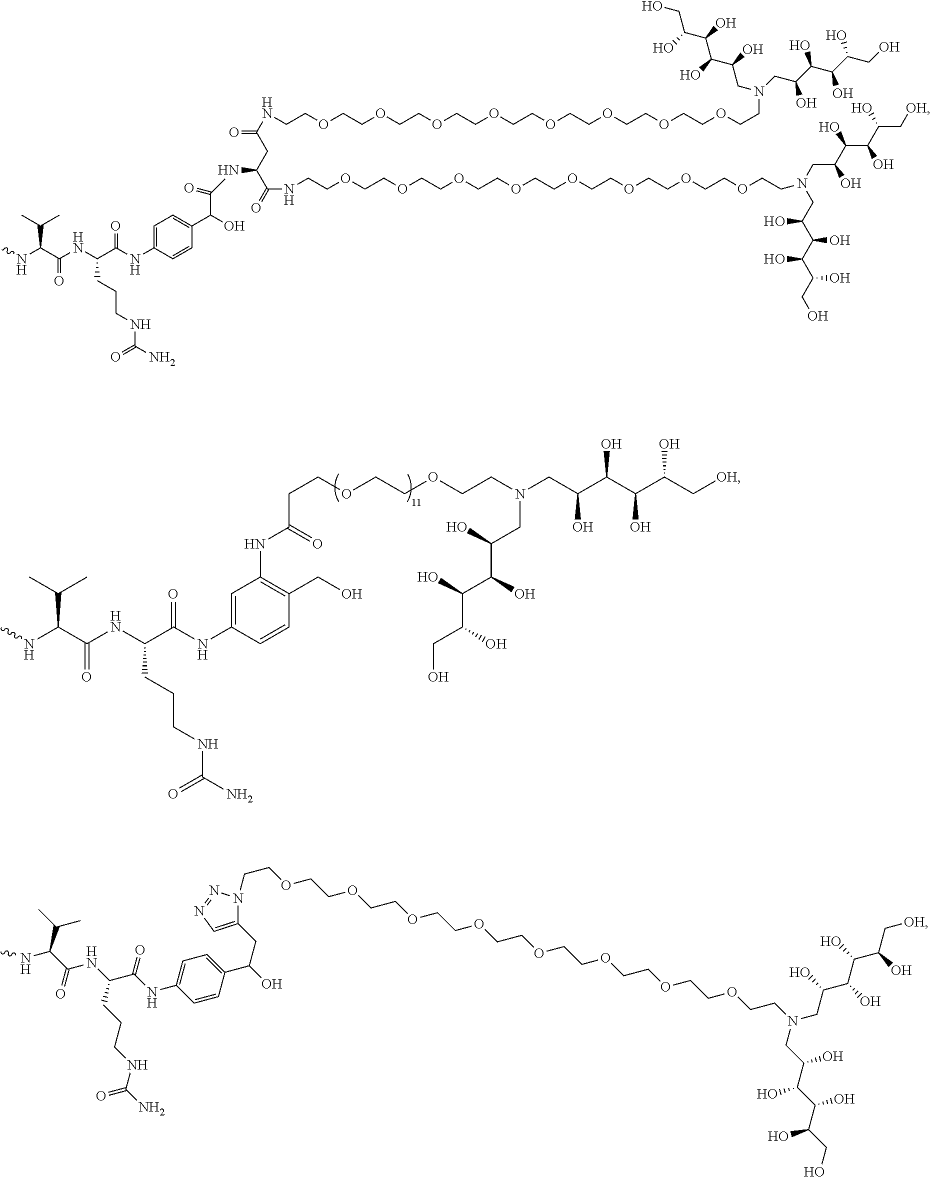

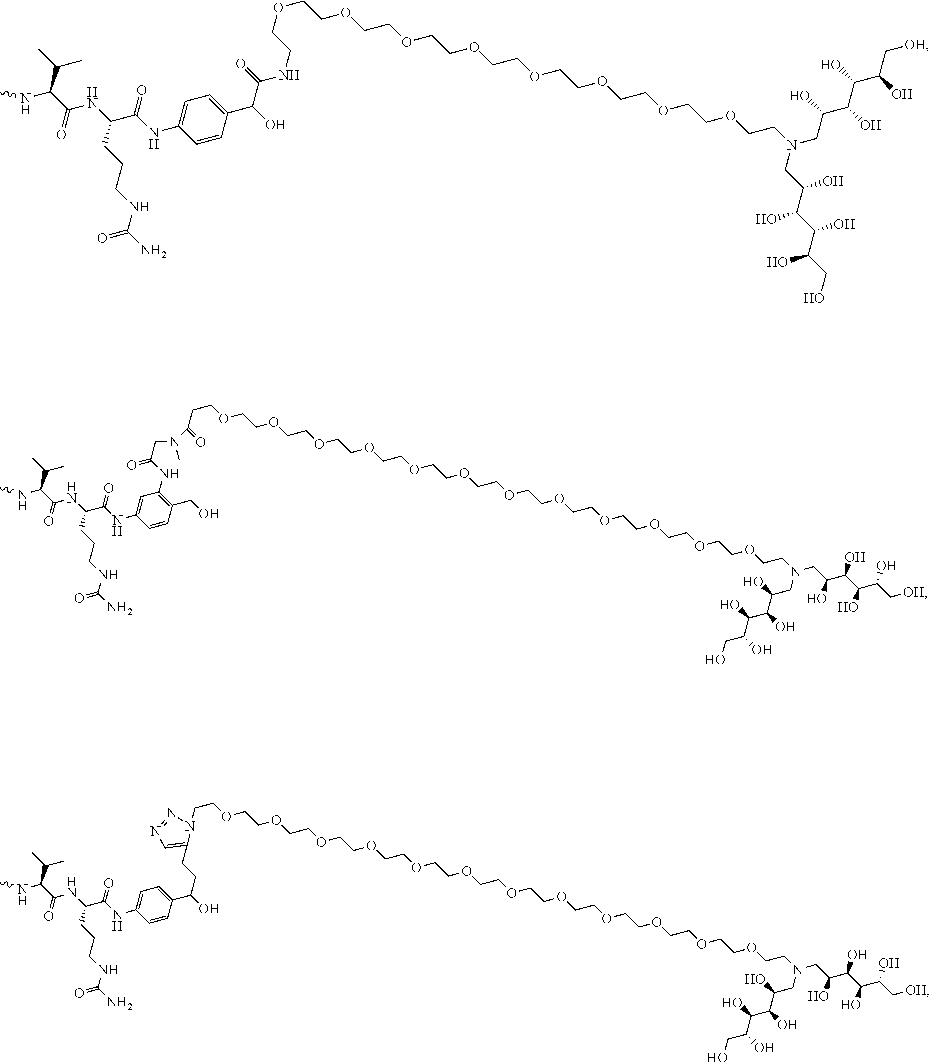

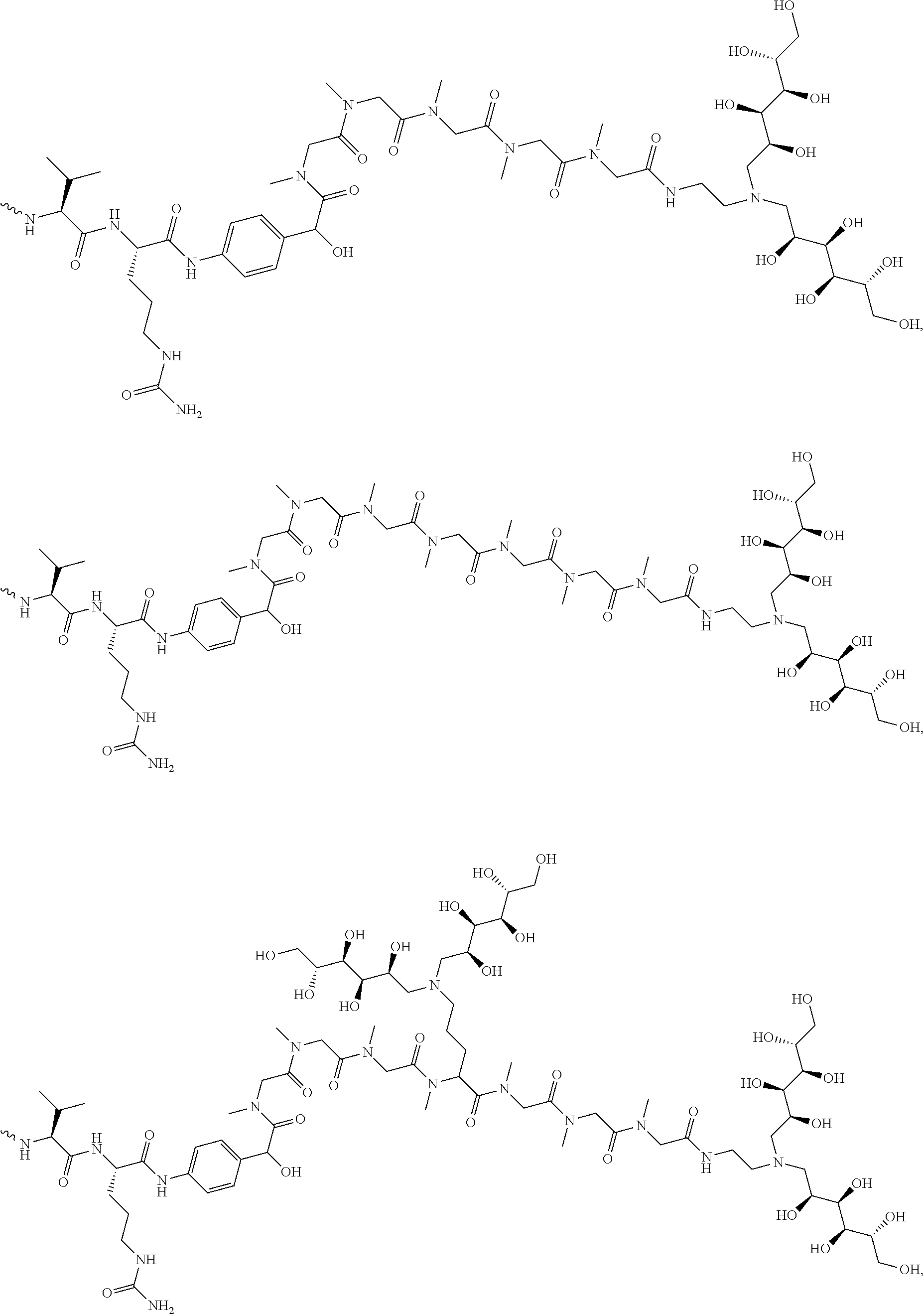

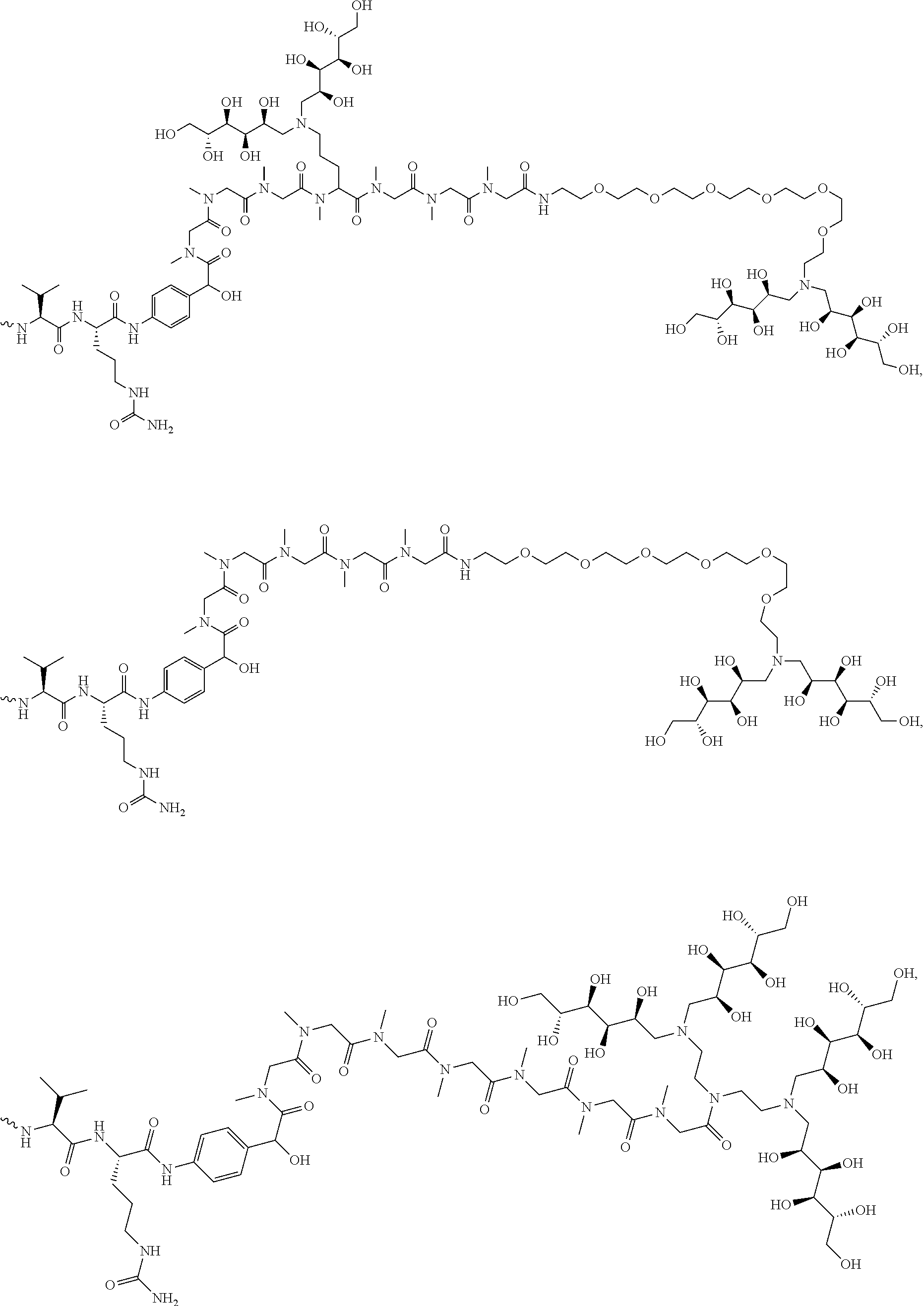

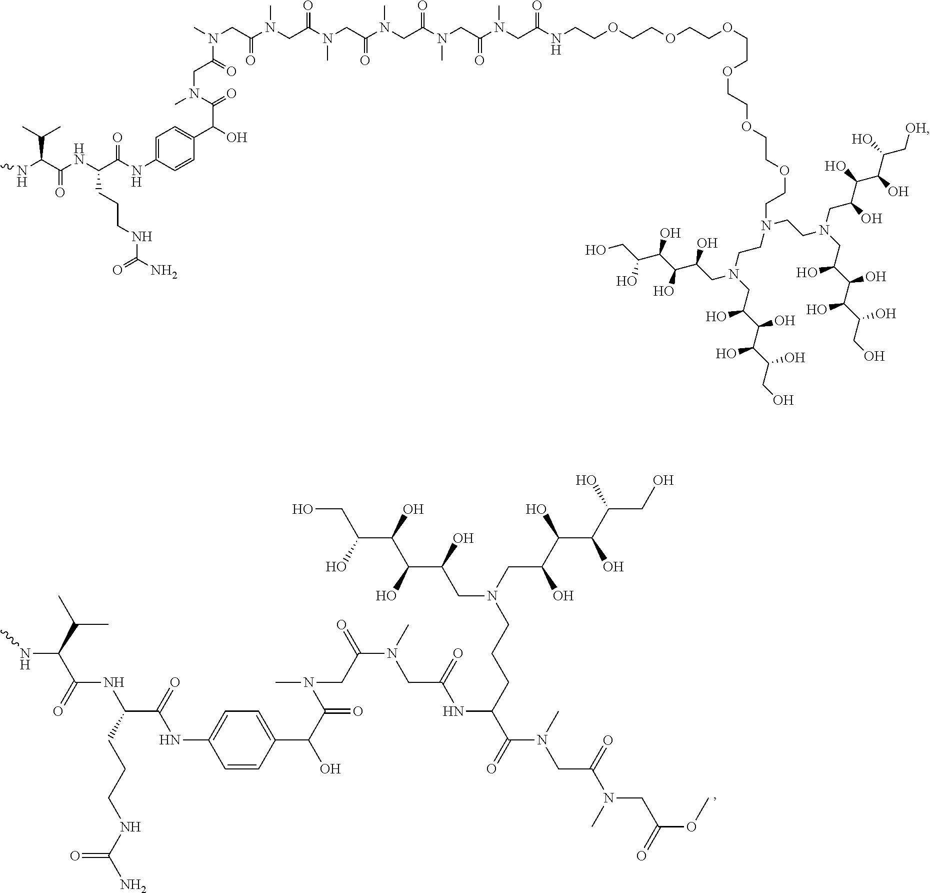

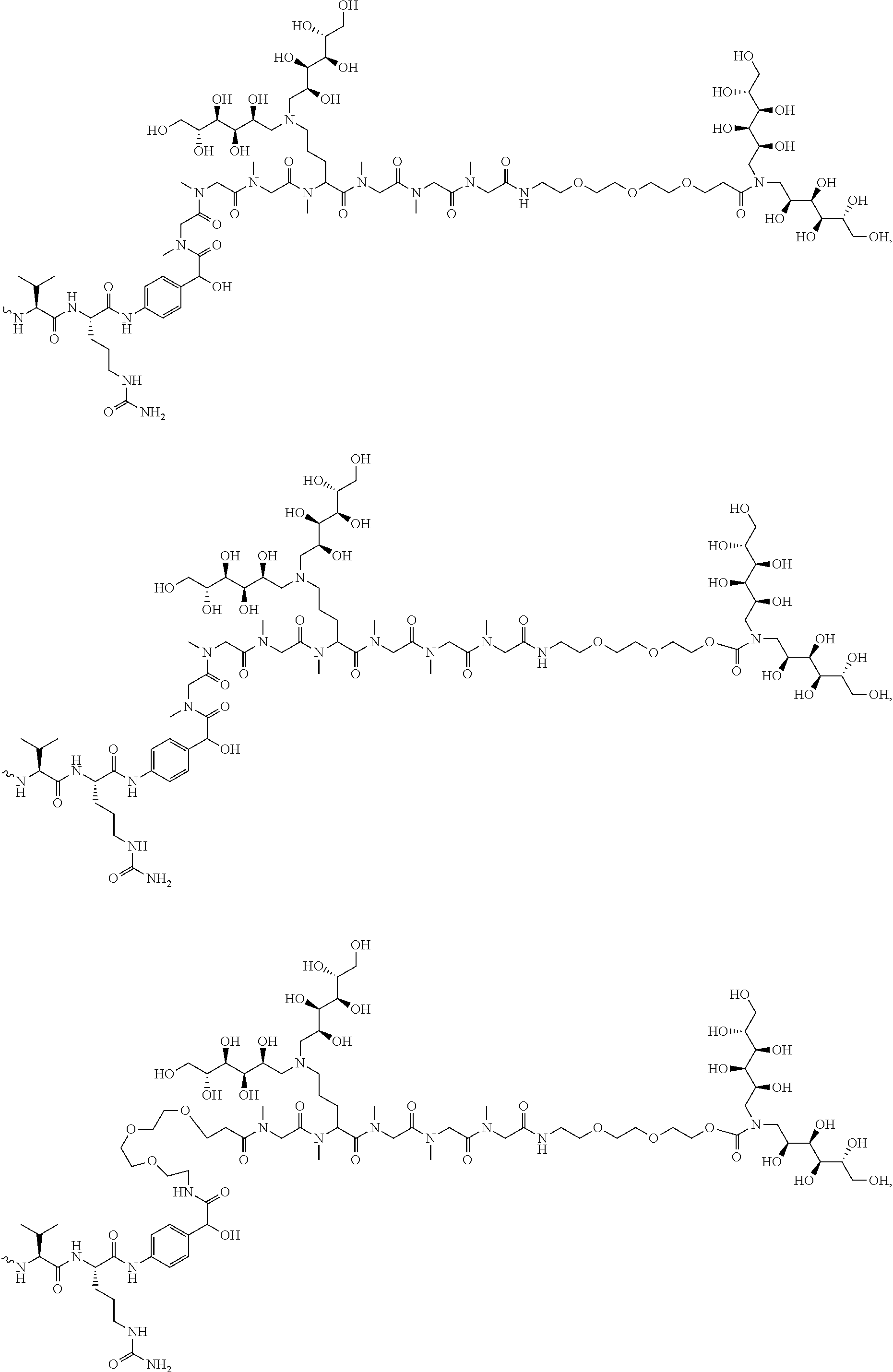

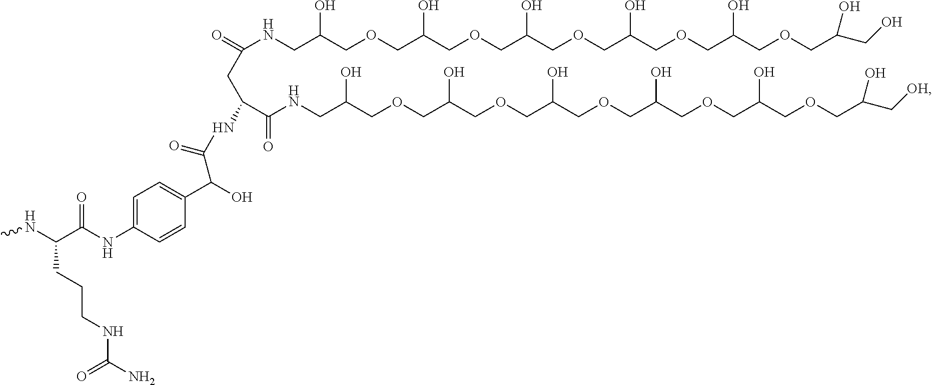

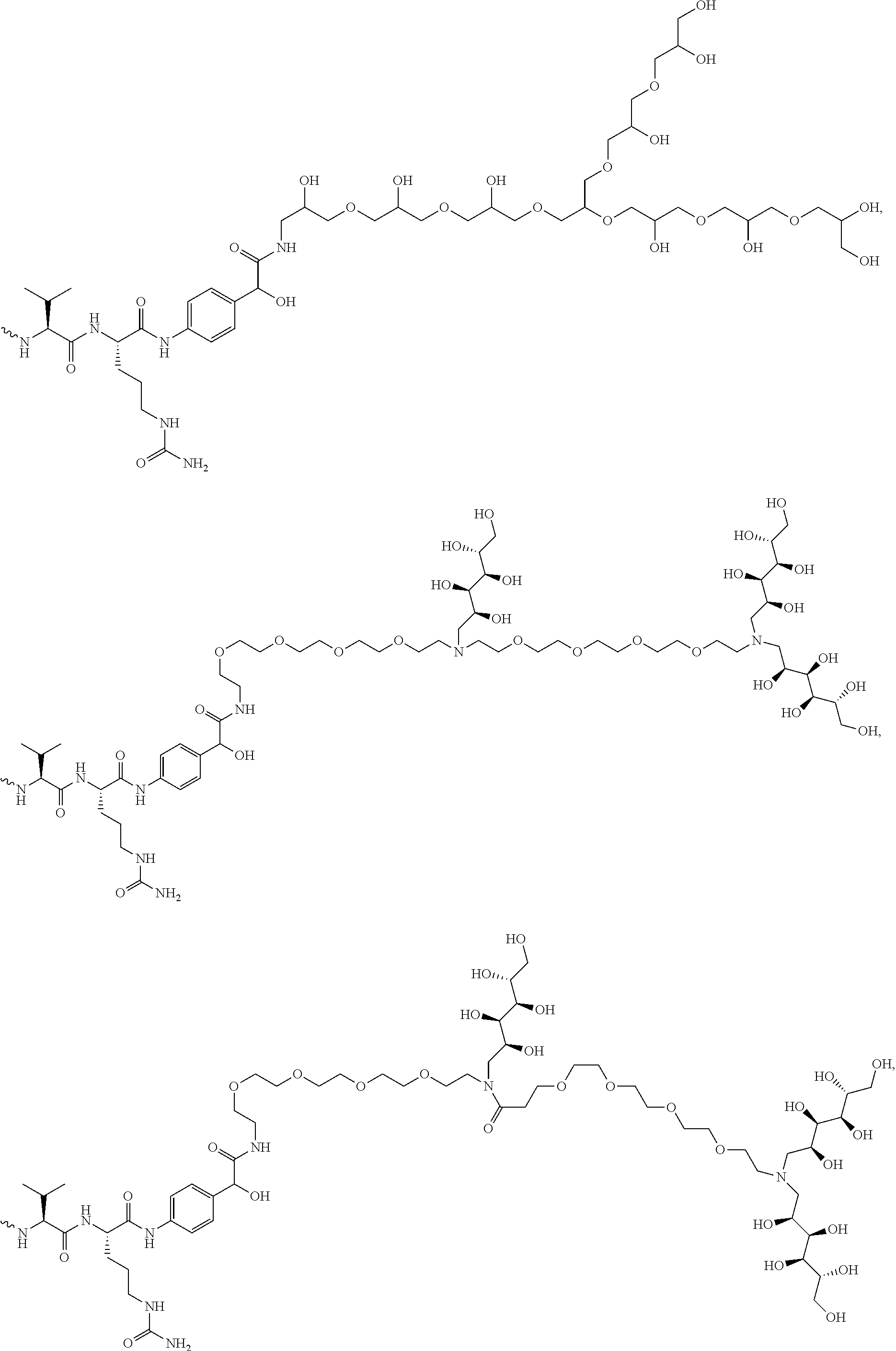

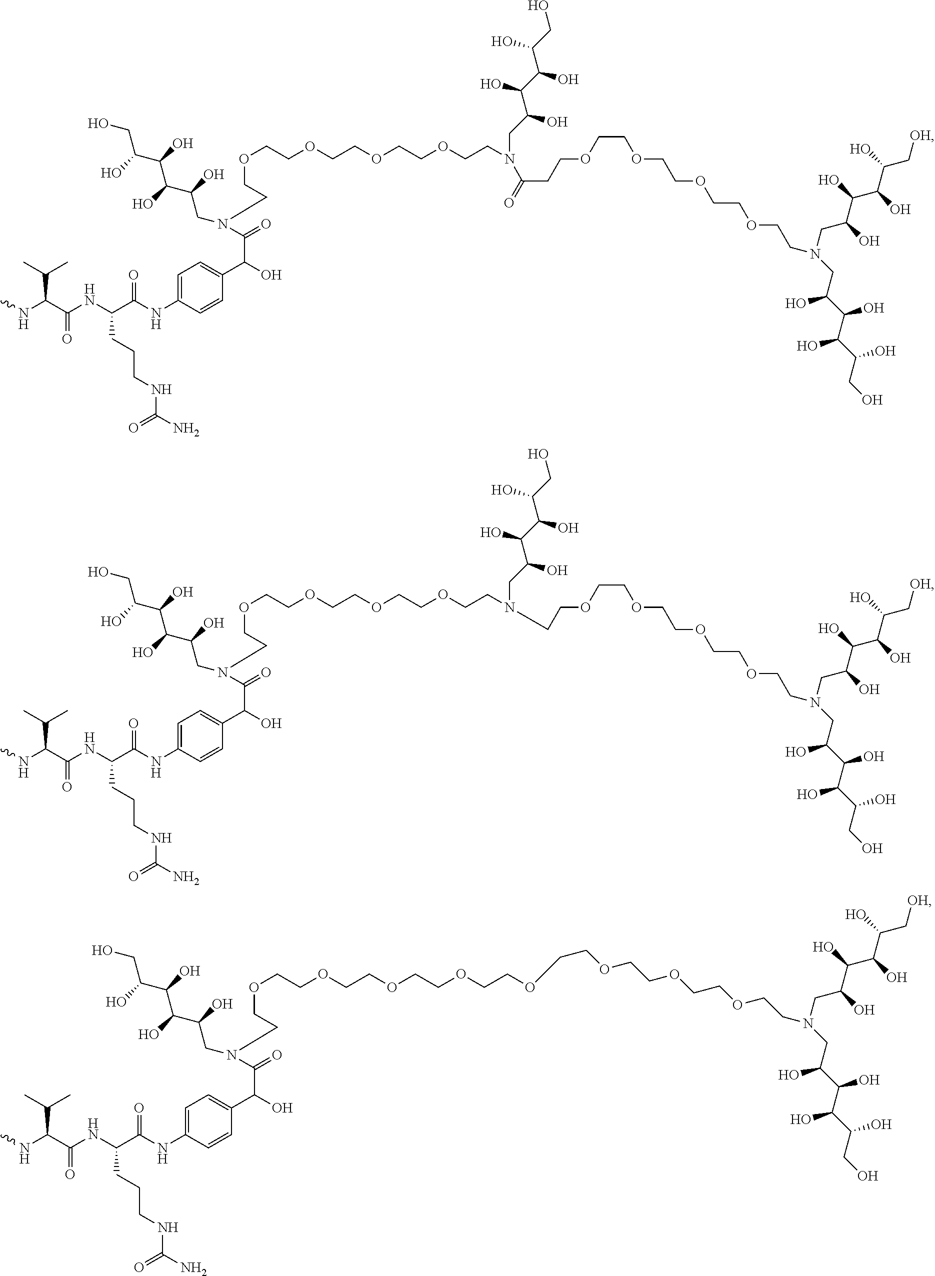

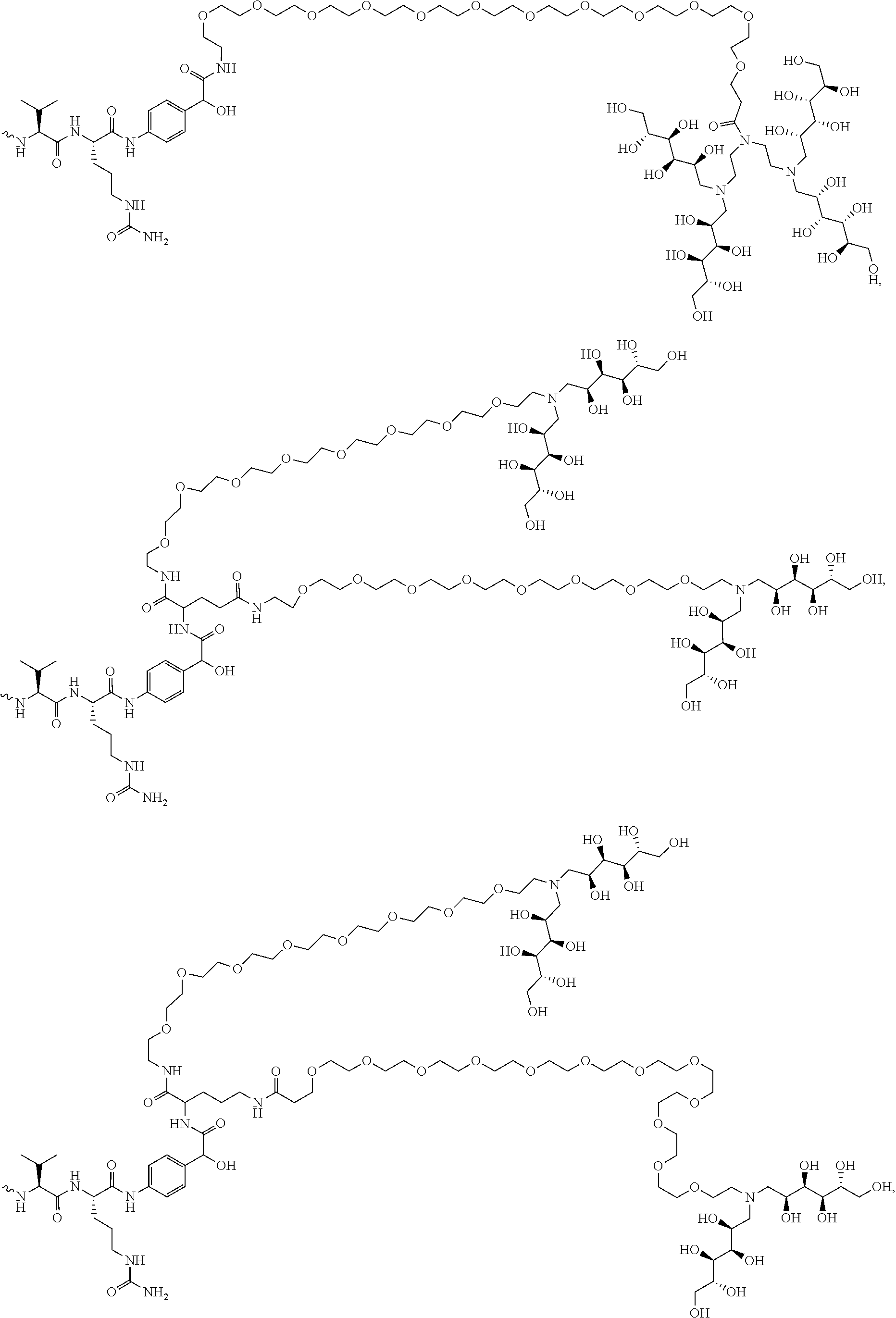

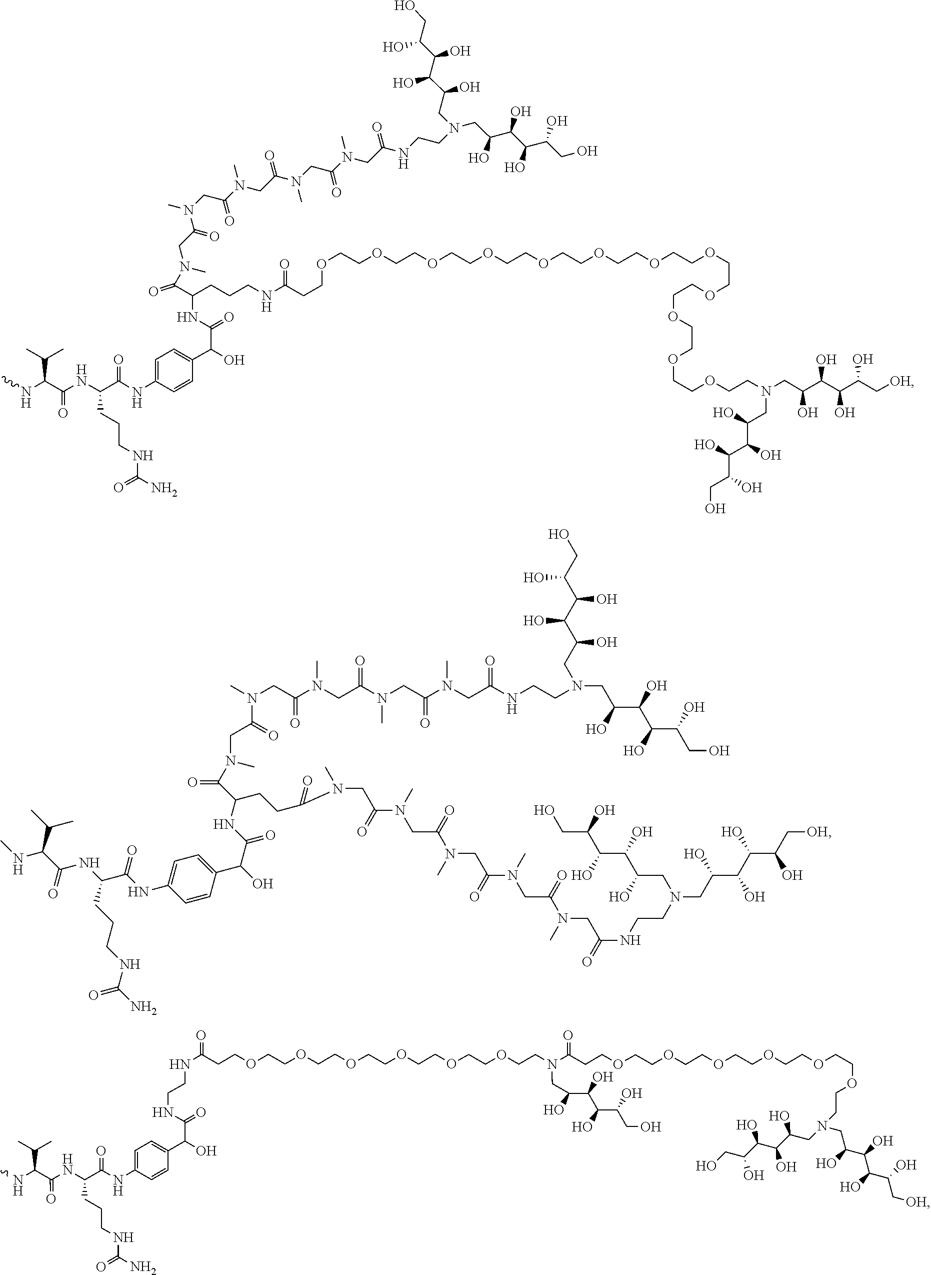

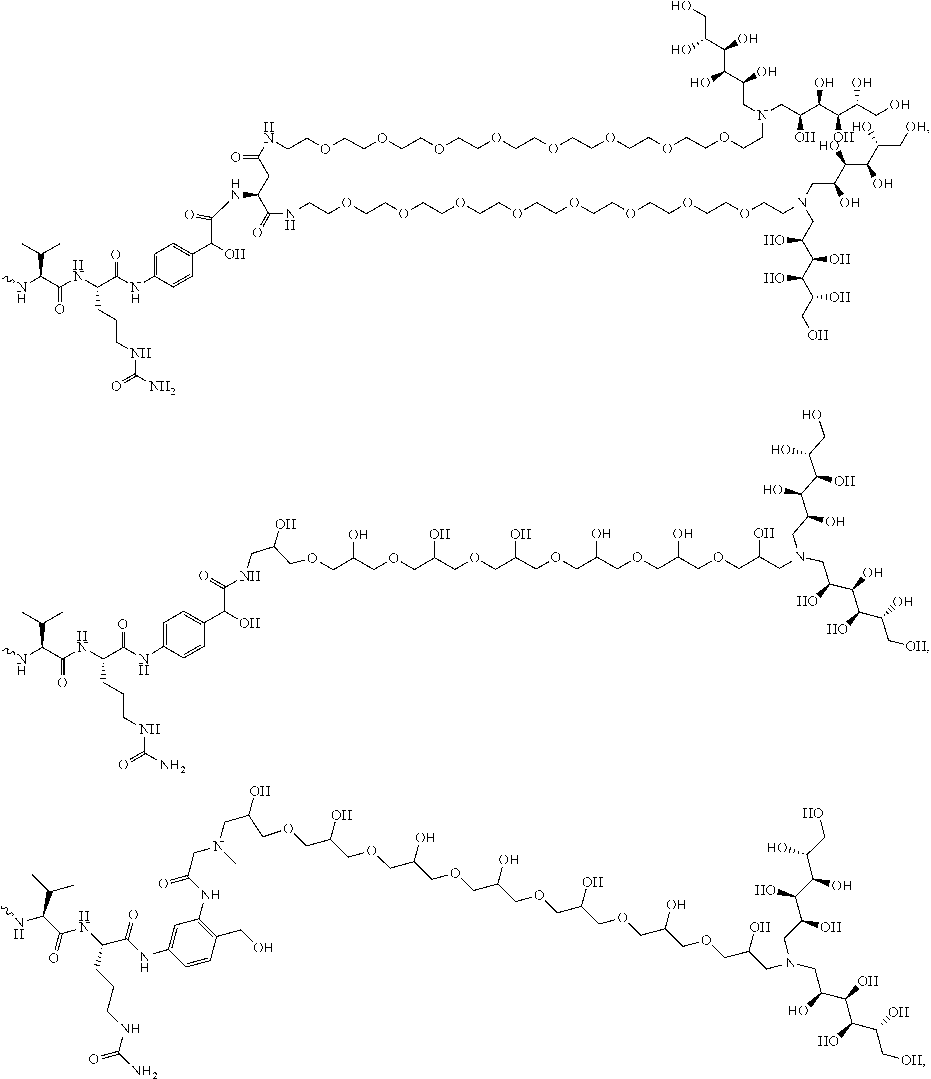

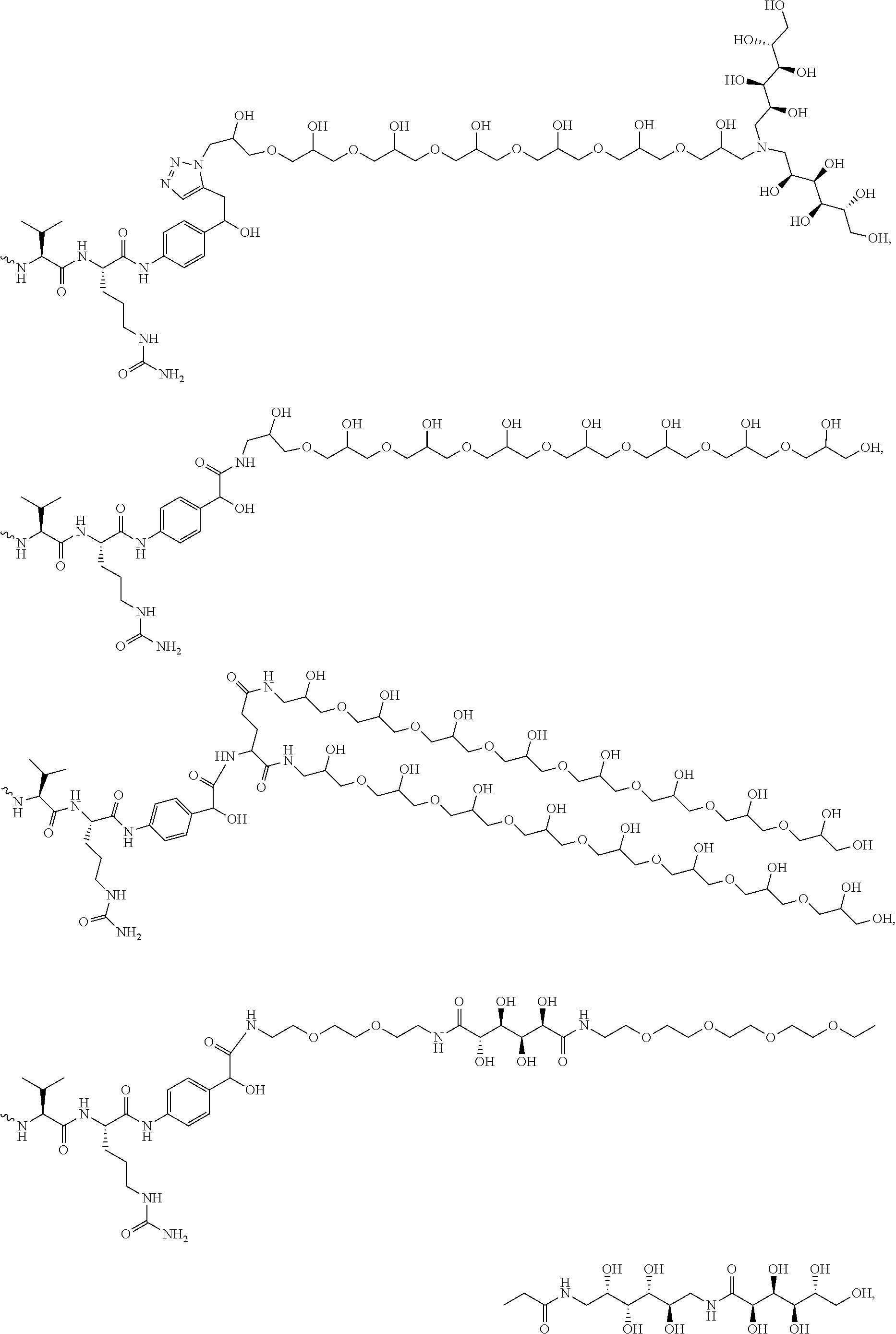

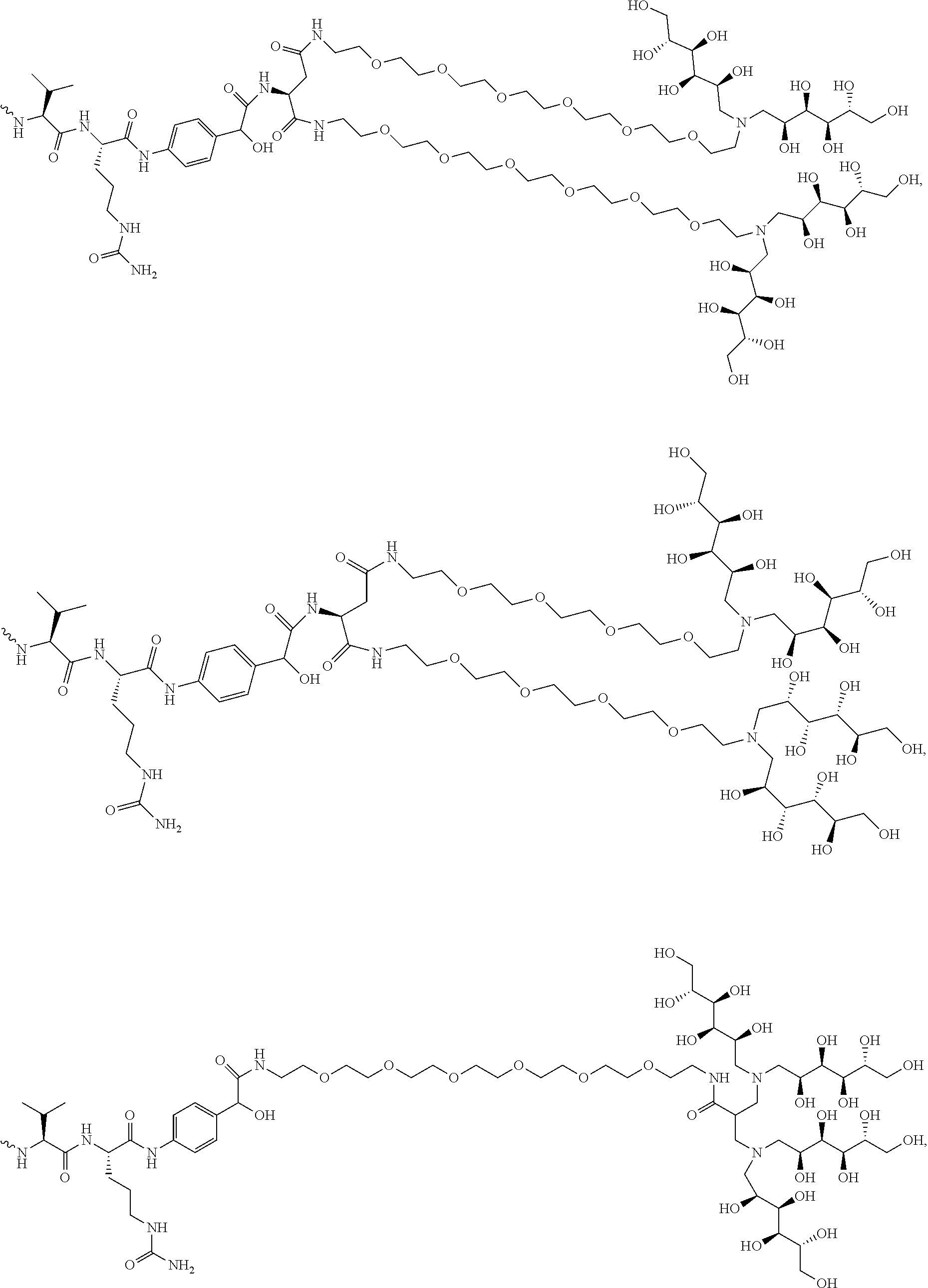

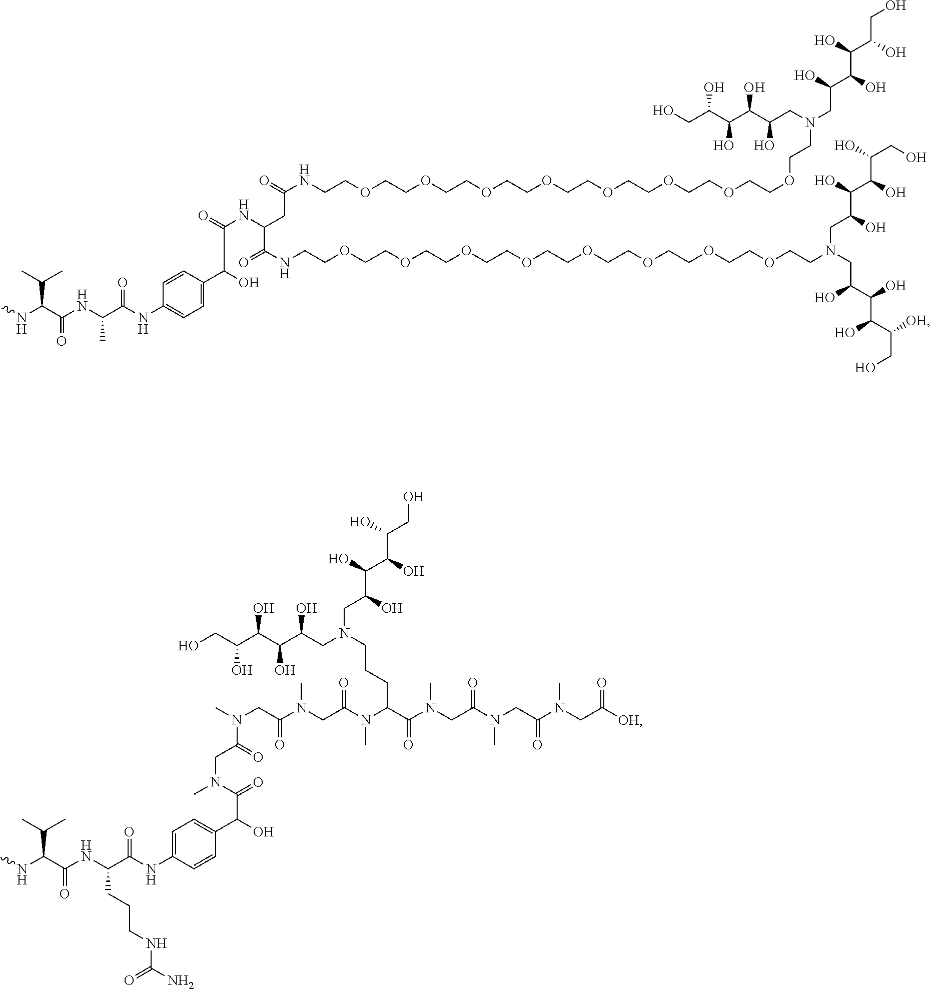

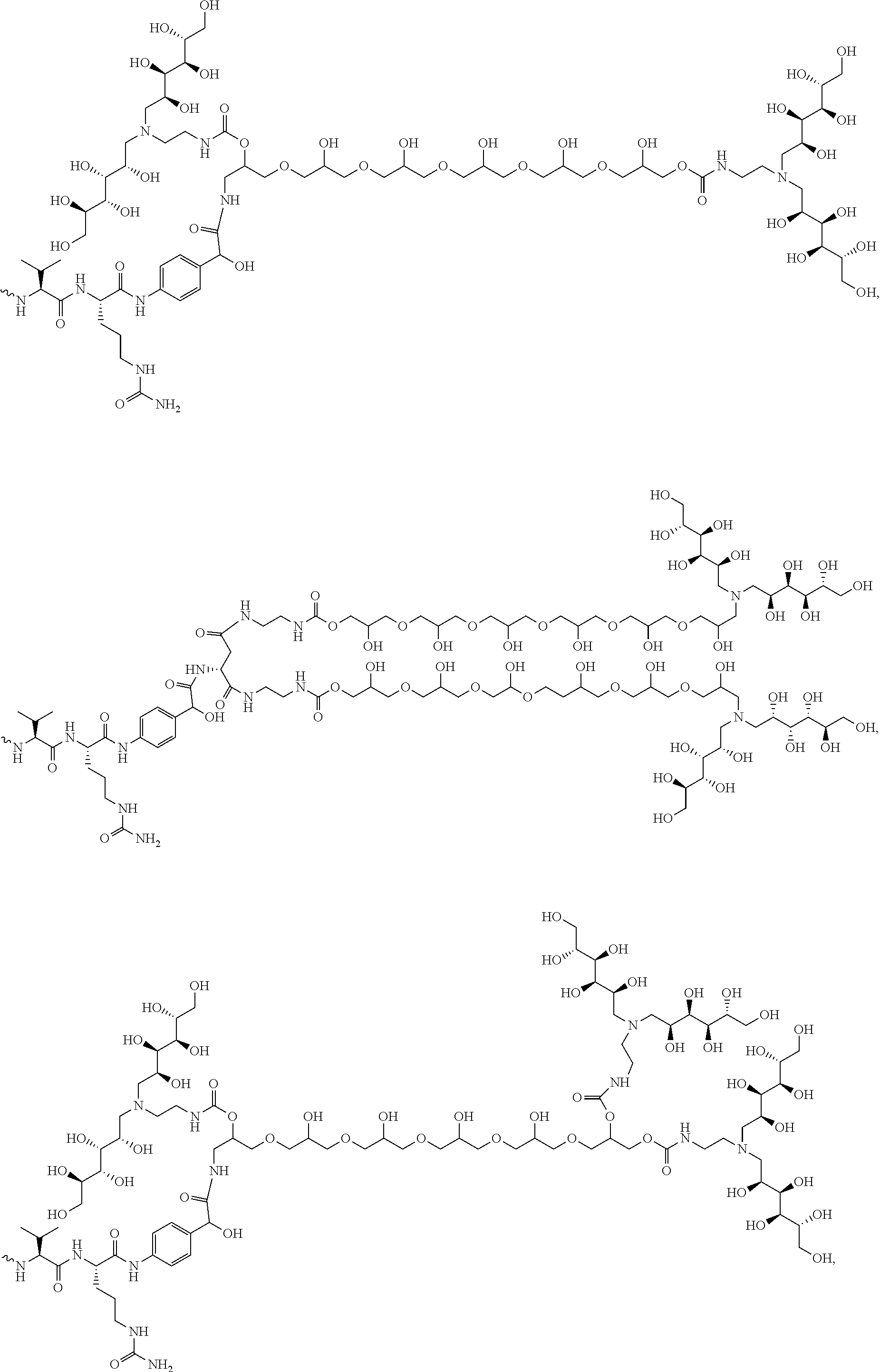

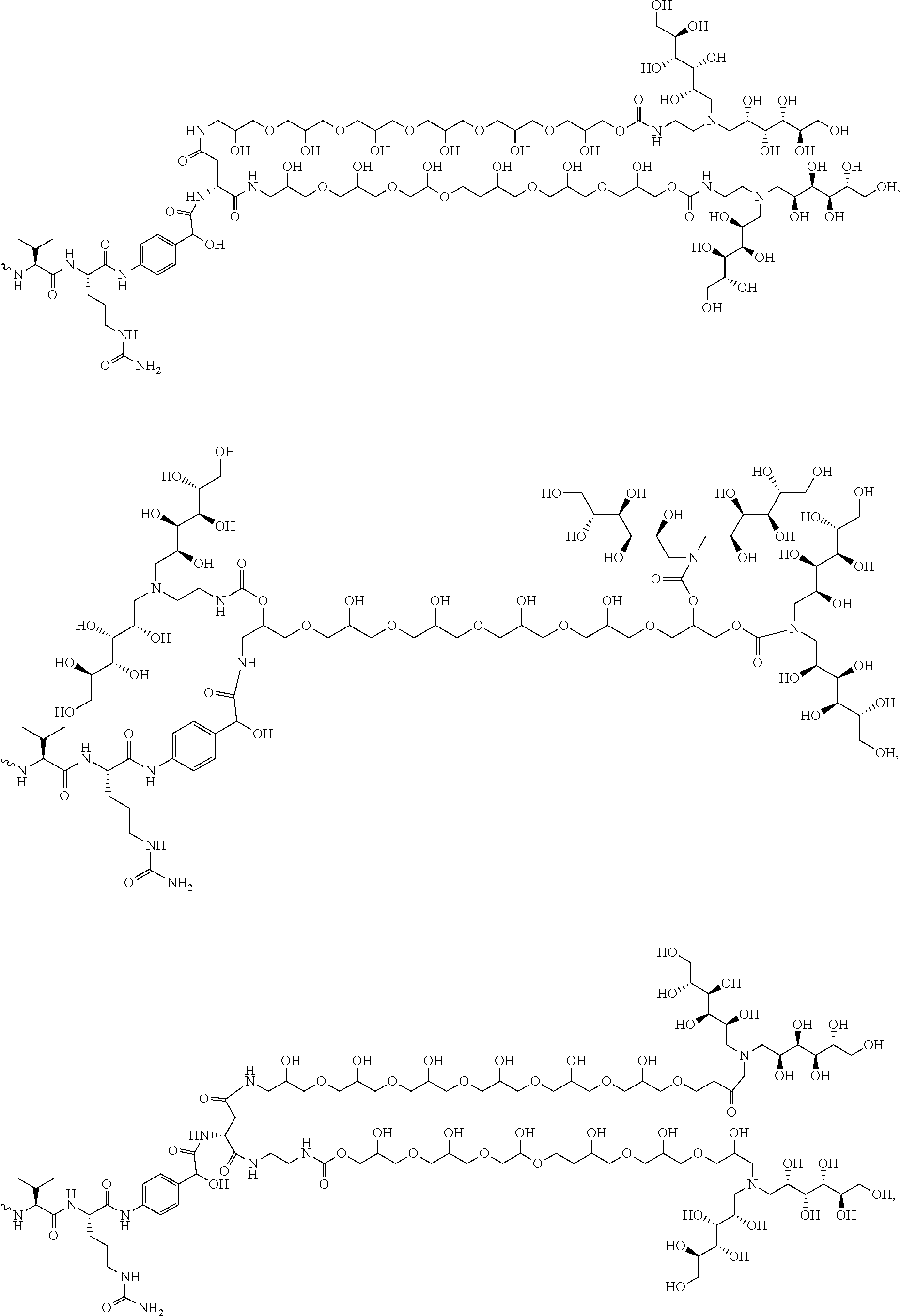

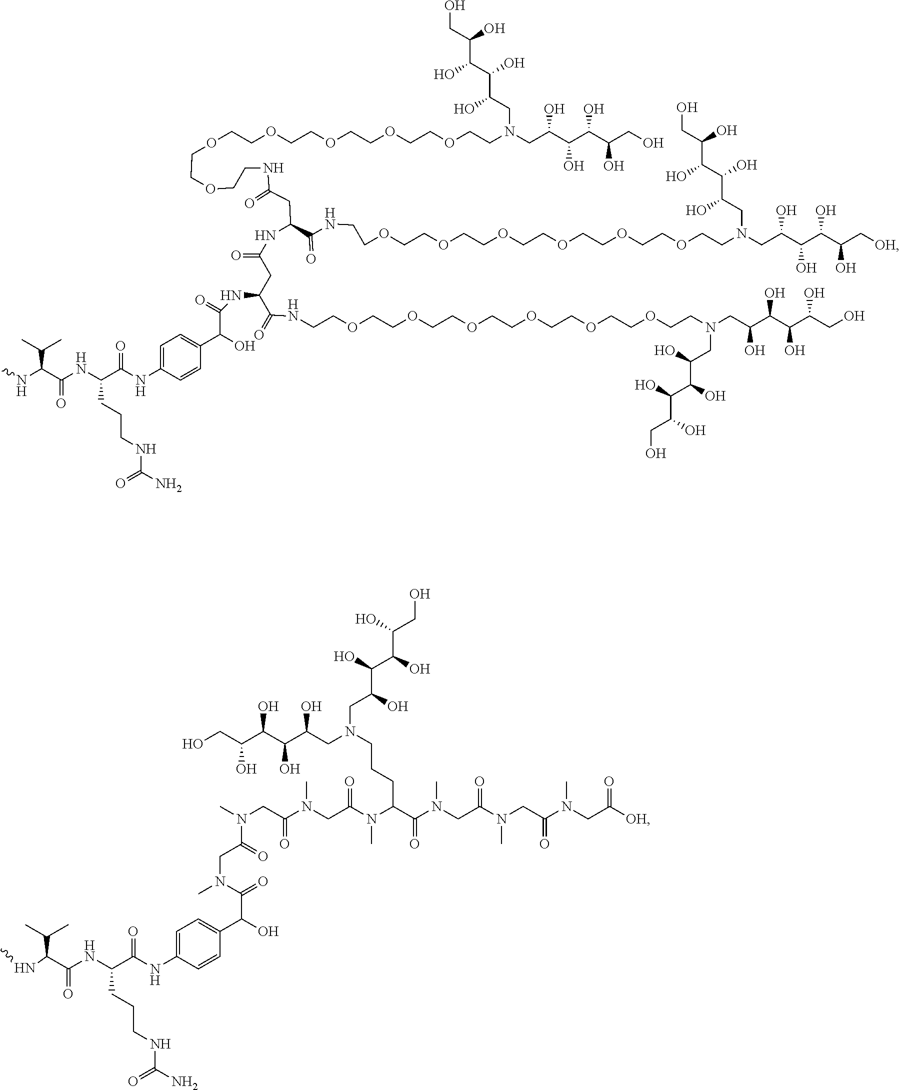

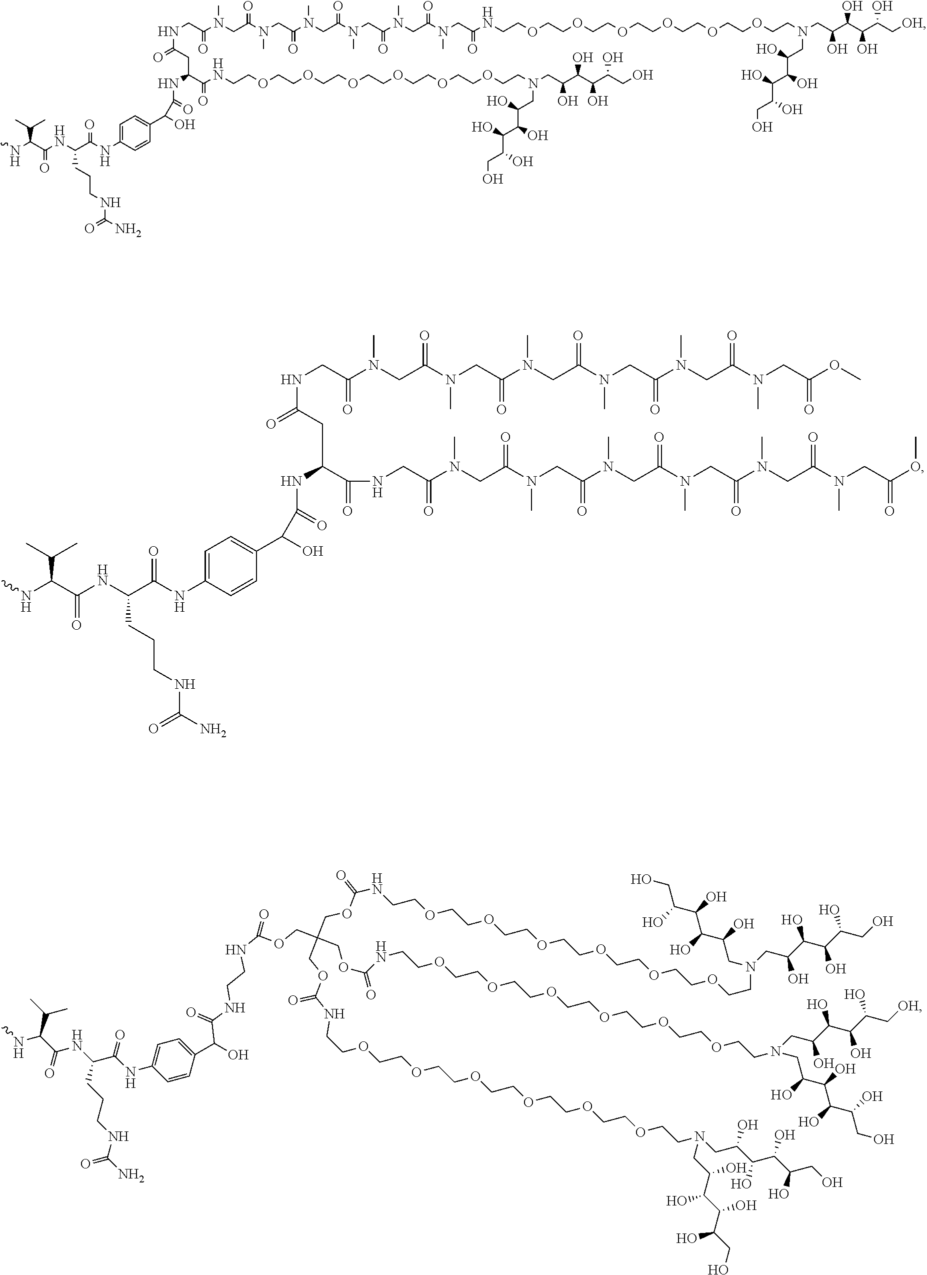

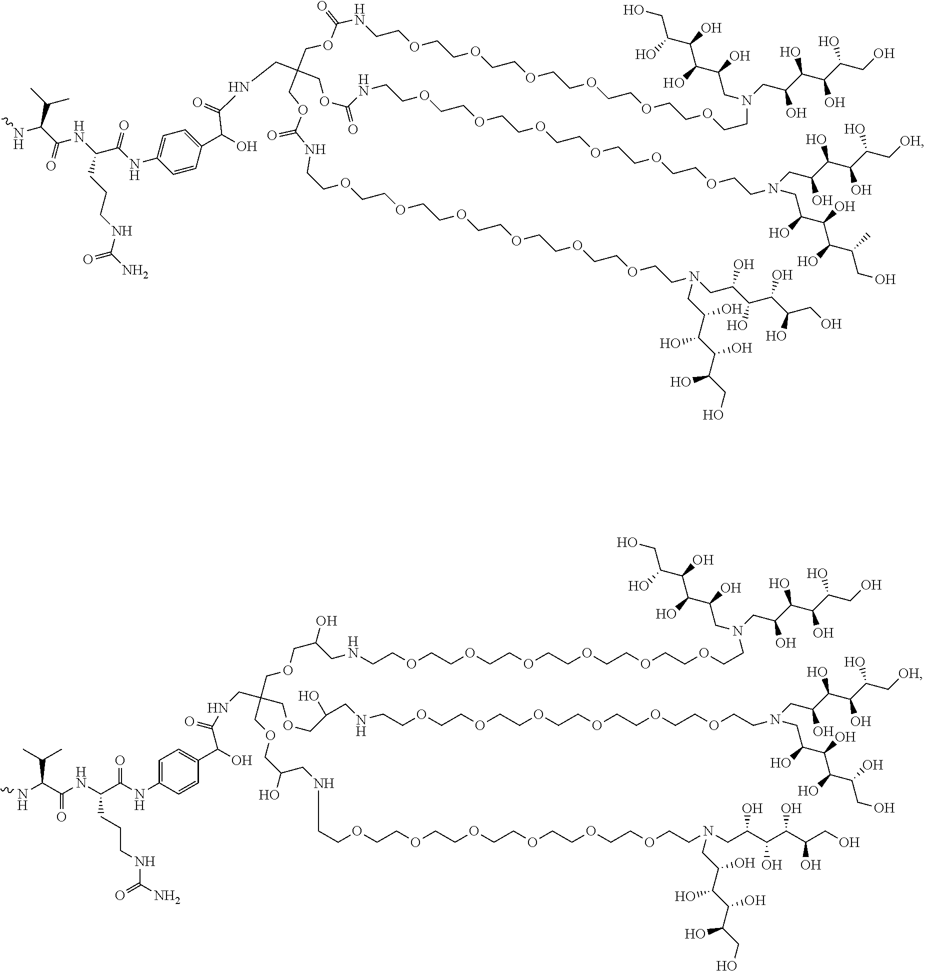

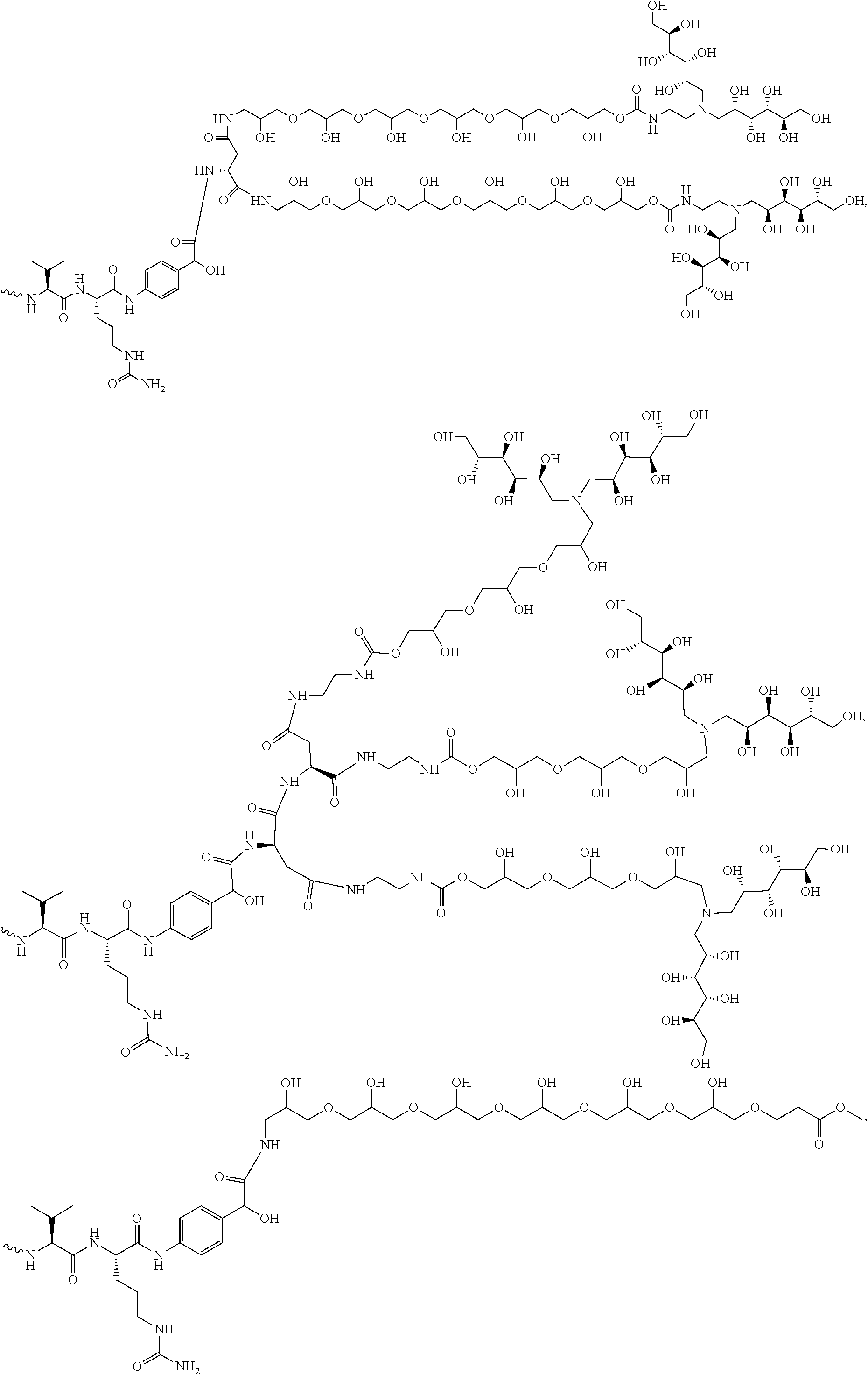

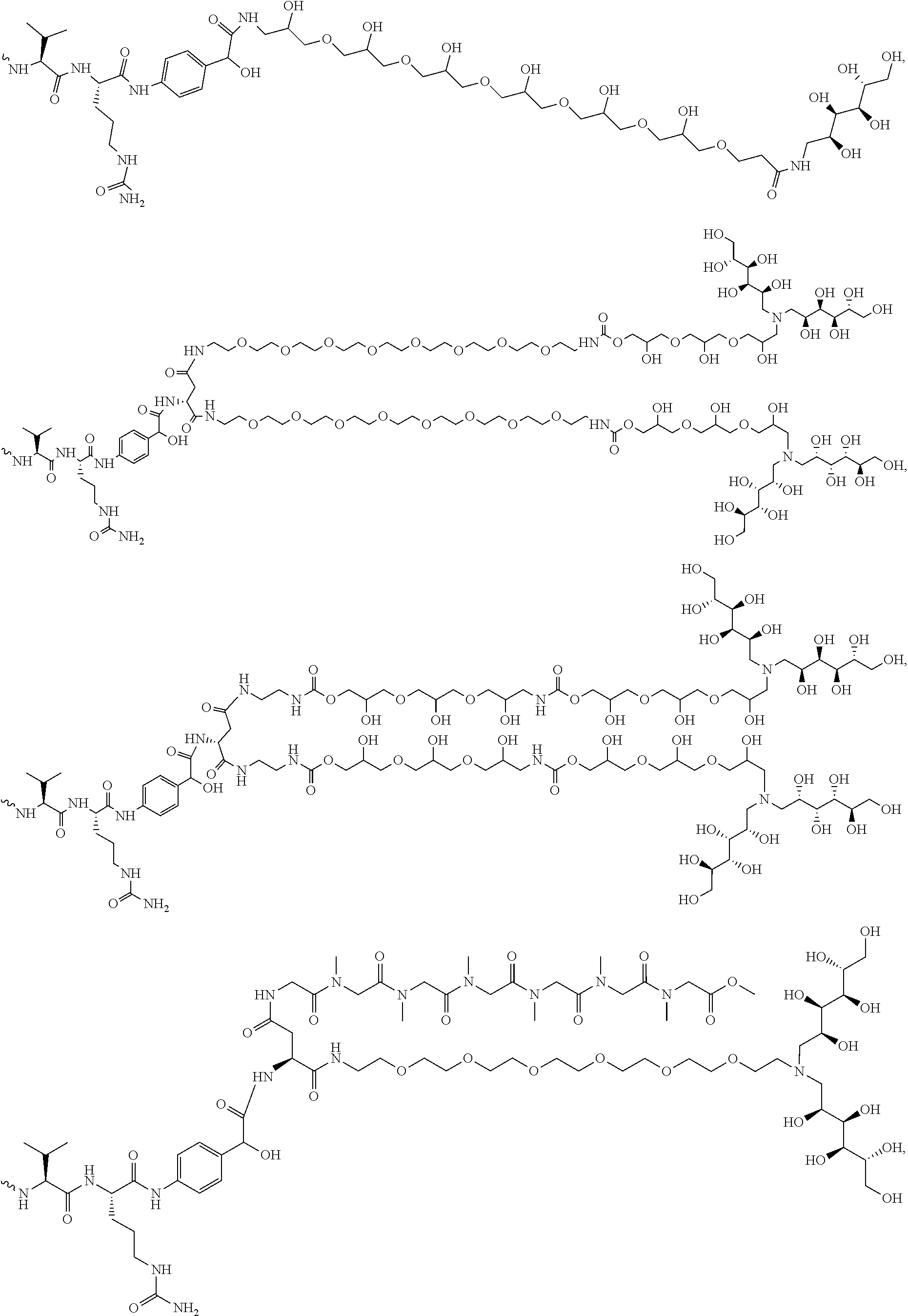

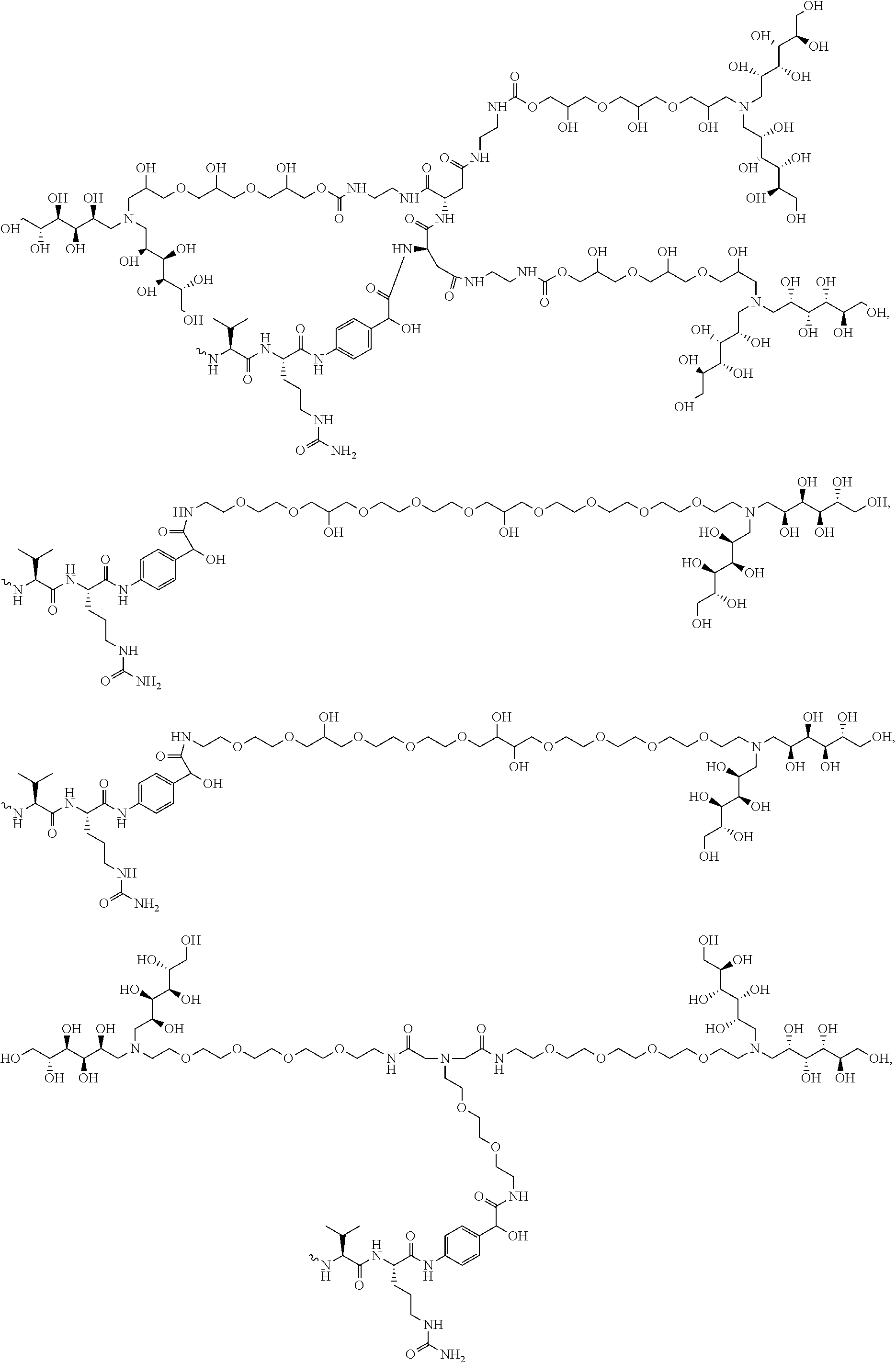

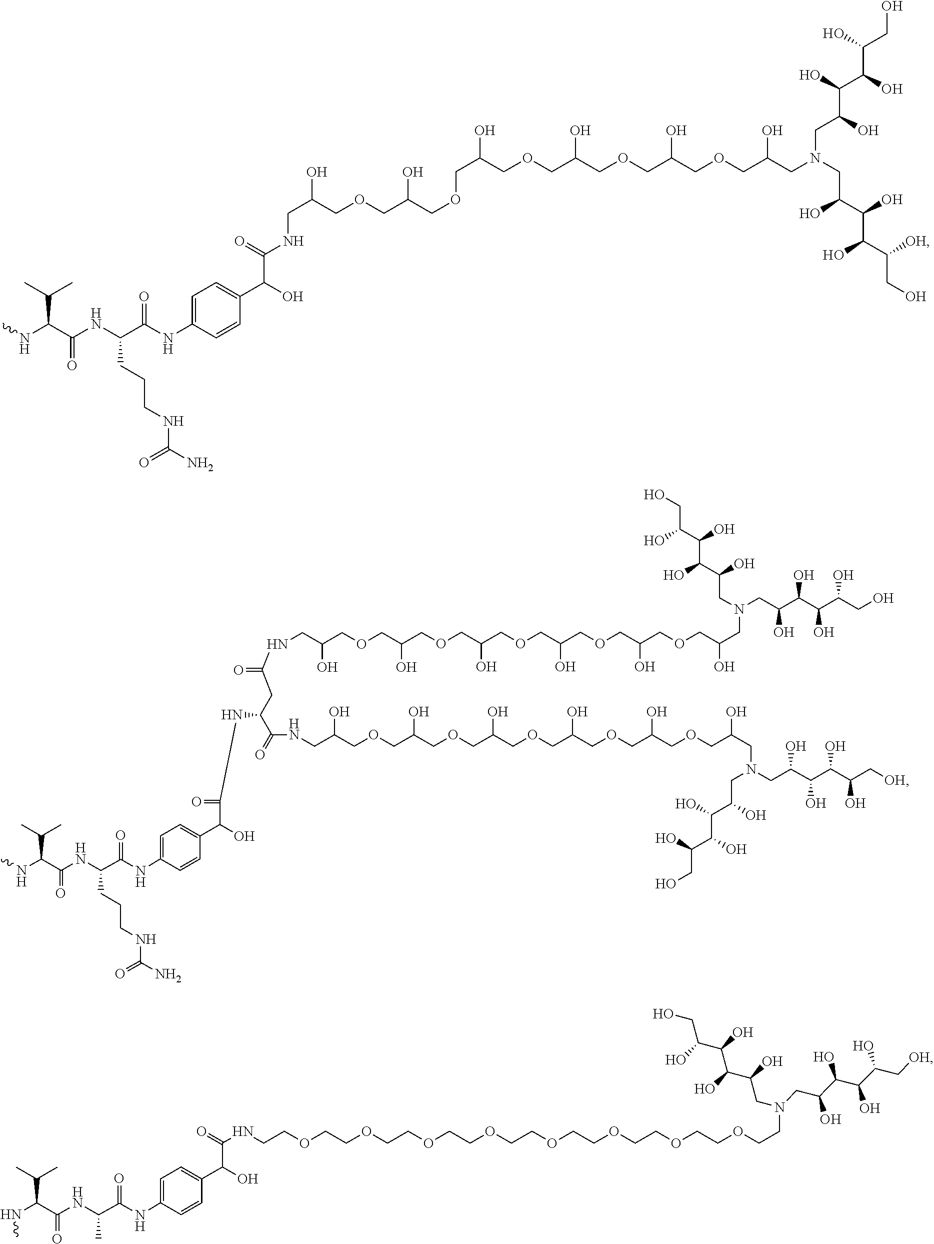

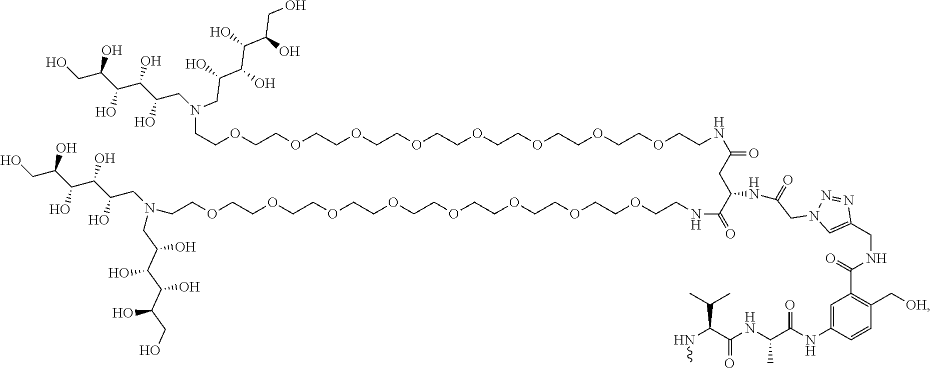

wherein the wavy line on the amino group indicates an attachment site to the Stretcher group; or, prior to attachment to the Stretcher group, indicates H, and the H on the benzylic OH is optionally replaced with a bond to at least one of the Drug units.

Embodiment 78. The Linker compound of any one of Embodiments 72 to 75, wherein the Linker unit is attached to a side chain of a subunit of the enzyme-cleavable group.

Embodiment 79. The Linker compound of any one of Embodiments 1 to 75, wherein the enzyme-cleavable group is joined to the Stretch Unit by a non-peptidic linking group.

Embodiment 80. The Linker compound of Embodiment 79, wherein the non-peptidic linking group is selected from optionally-substituted C₁-C₁₀ alkylene, optionally-substituted C₂-C₁₀ alkenylene, optionally-substituted C₂-C₁₀ alkynylene, or optionally-substituted polyethylene glycol.

Embodiment 81. The Linker compound of any one of Embodiments 1-80, comprising the Stretcher group attached to the enzyme-cleavable group.

Embodiment 82. The Linker compound of Embodiment 81, wherein the Stretcher group is selected from the following:

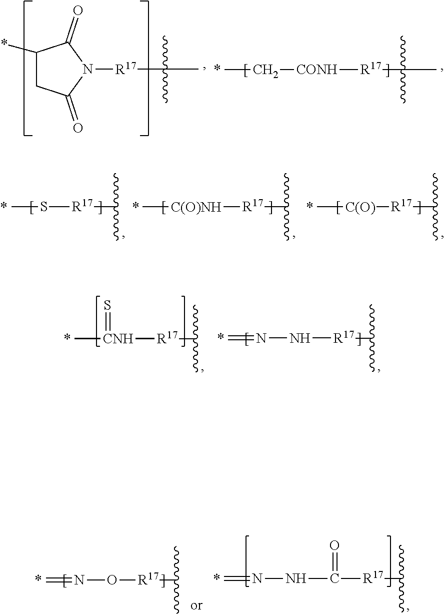

wherein R¹⁷ is —C₁-C₁₀ alkylene-, —C₁-C₁₀ heteroalkylene-, —C₃-C₈ carbocyclo-, —O—(C₁-C₈ alkylene)-, —(CH₂—O—CH₂)_{b}—C₁-C₈ alkylene- (where b is 1 to 26), —C₁-C₈ alkylene-(CH₂—O—CH₂)_{b}— (where b is 1 to 26), —C₁-C₈ alkylene-(CH₂—O—CH₂)_{b}—C₁-C₈ alkylene- (where b is 1 to 26), -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁(alkylene-(C₃-C₈ heterocyclo)-, —(C₃-C₈ heterocyclo)-C₁-C₁₀ alkylene-, —C₁-C₁₀ alkylene-C(═O)—, —C₁-C₁₀alkylene-C(O)NH—C₁-C₈alkylene-[O—CH₂—CH₂]ₙ—C(O)— (where n is 1 to 26), C₁-C₁₀ heteroalkylene-C(═O)—, —C₁-C₁₀ alkylene-(CH₂—O—CH₂)_{b}—C(═O)— (where b is 1 to 26), —(CH₂—O—CH₂)_{b}—C₁-C₈ alkylene-C(═O)— (where b is 1 to 26), —C₁-C₈ alkylene-(CH₂—O—CH₂)_{b}—C₁-C₈ alkylene-C(═O)— (where b is 1 to 26), —C₃-C₈ carbocyclo-C(═O)—, —O—(C₁-C₈ alkyl)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, —C₁-C₁₀ heteroalkylene-NH—, —C₁-C₈ alkylene-(CH₂—O—CH₂)_{b}—NH— (where b is 1 to 26), —(CH₂—O—CH₂)_{b}—C₁-C₈ alkylene-NH— (where b is 1 to 26), —C₁-C₈ alkylene-(CH₂—O—CH₂)_{b}—C₁-C₈ alkylene-NH— (where b is 1 to 26), —C₁-C₈ alkylene-(C(═O))—NH—(CH₂—O—CH₂)_{b}—C(═O)— (where b is 1 to 26), —C₁-C₈ alkylene-(C(═O))—NH—(CH₂—O—CH₂)_{b}—C₁-C₈ alkylene-C(═O)— (where b is 1 to 26), —C₁-C₈ alkylene-NH—(C(═O))—(CH₂—O—CH₂)_{b}—NH— (where b is 1 to 26), —C₁-C₈ alkylene-NH—(C(═O))—(CH₂—O—CH₂)_{b}—C₁-C₈ alkylene-NH— (where b is 1 to 26), —C₃-C₈ carbocyclo-NH—, —O—(C₁-C₈ alkyl)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkyl)-S—, -arylene-S—, —C₁-C₁(alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—; or
wherein the Stretcher group comprises maleimido(C₁-C₁₀ alkylene-C(O)—, maleimido(CH₂OCH₂)p₂(C₁-C₁₀ alkyene)C(O)—, maleimido(C₁-C₁₀ alkyene) (CH₂OCH₂)ₚ₂C(O)—, or a ring open form thereof, wherein p2 is from 1 to 26;
and wherein * is an attachment site to the Targeting group, and the wavy line is an attachment site to the enzyme-cleavable group.

Embodiment 83. The Linker compound of Embodiment 81, wherein the Stretcher group is selected from the following:

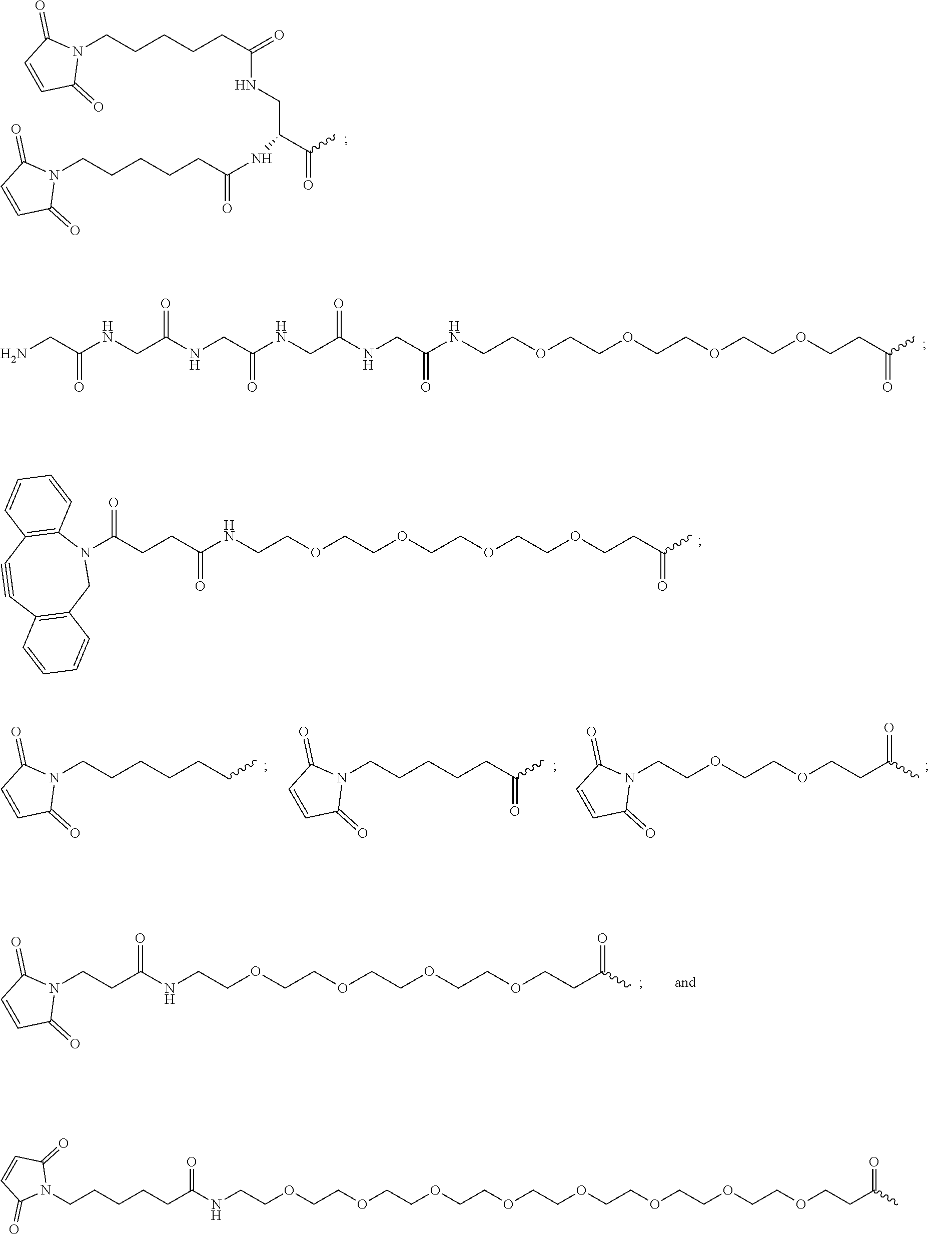

wherein the wavy line [symbol] indicates an attachment site of the Stretcher group to the enzyme-cleavable group, and the attachment site to the Targeting group is on the maleimide, primary amine or alkyne functional group.

Embodiment 84. The Linker compound of Embodiment 1, having one of the following structures:

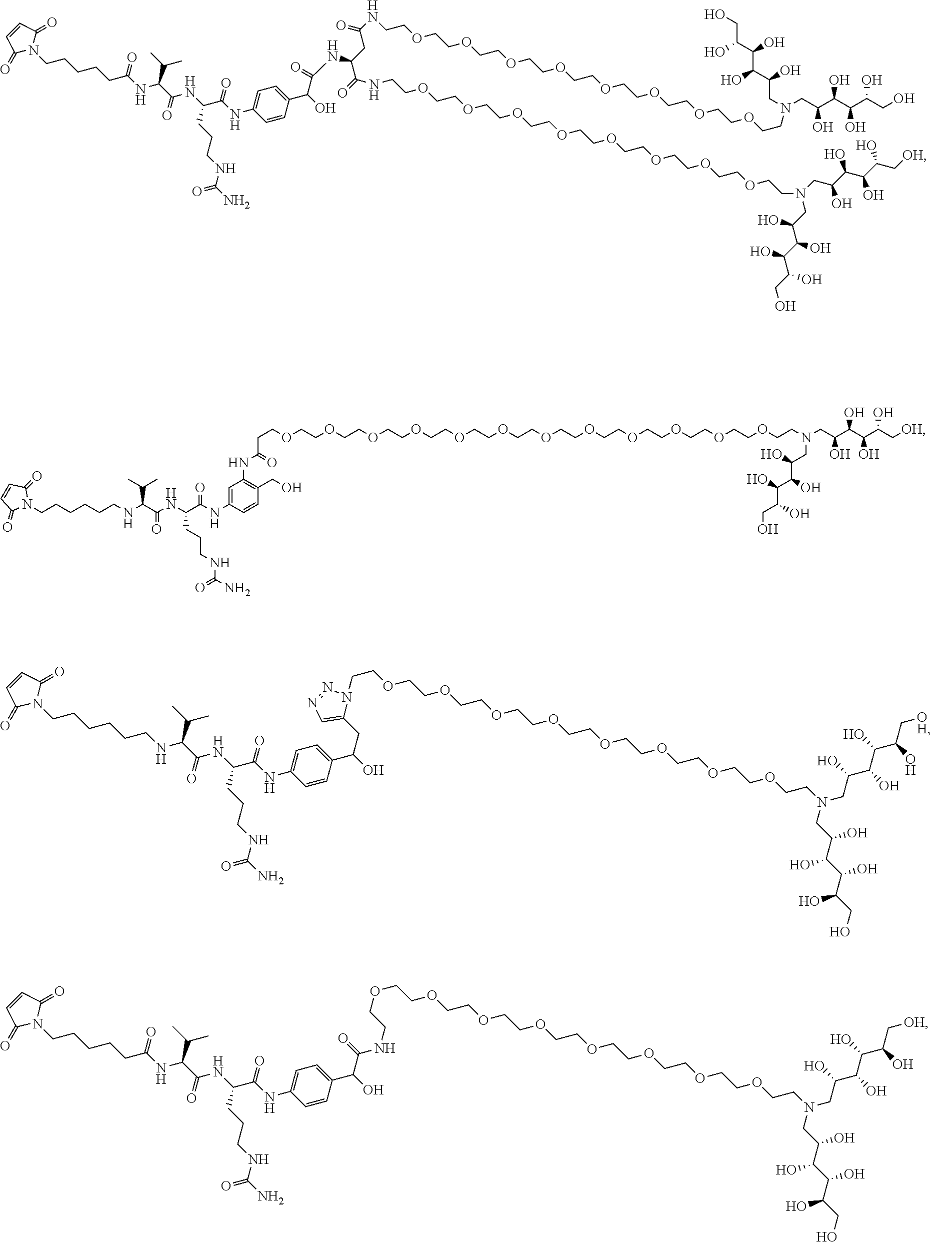

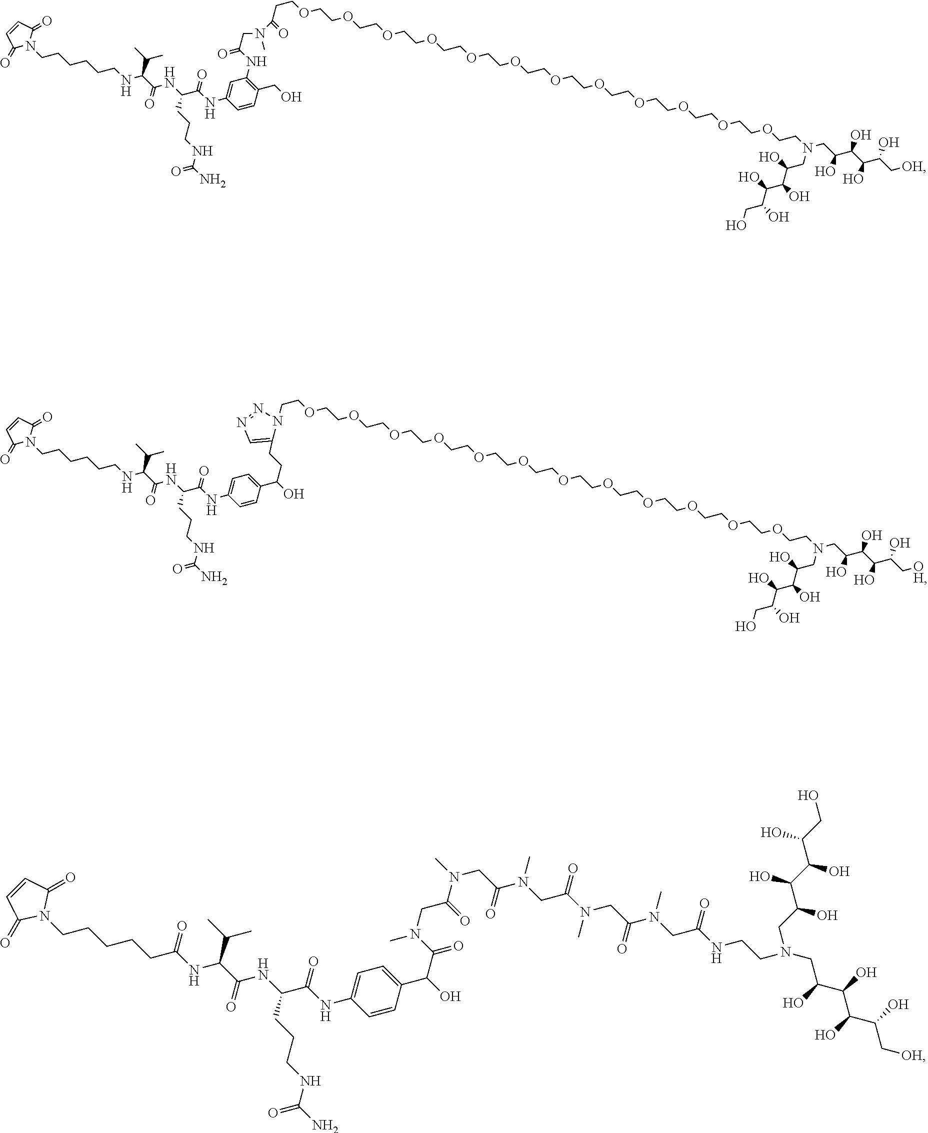

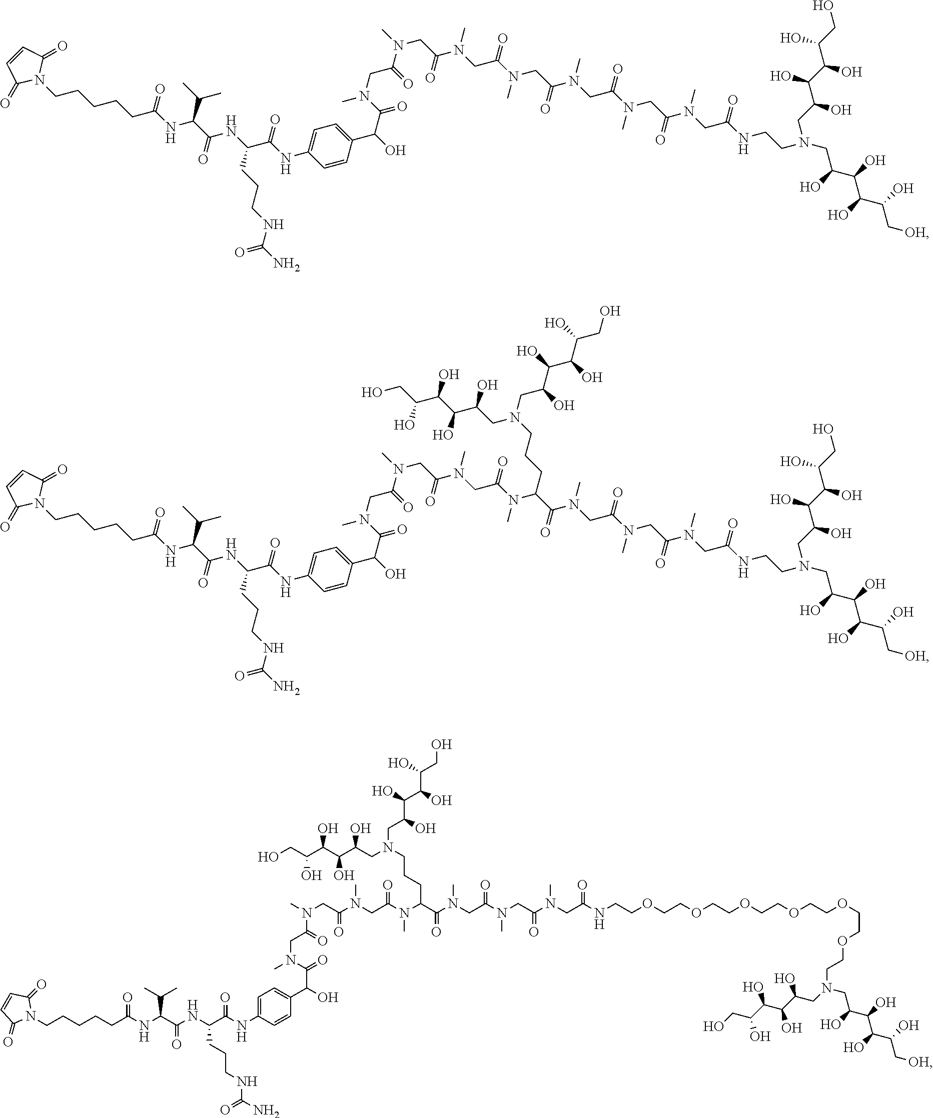

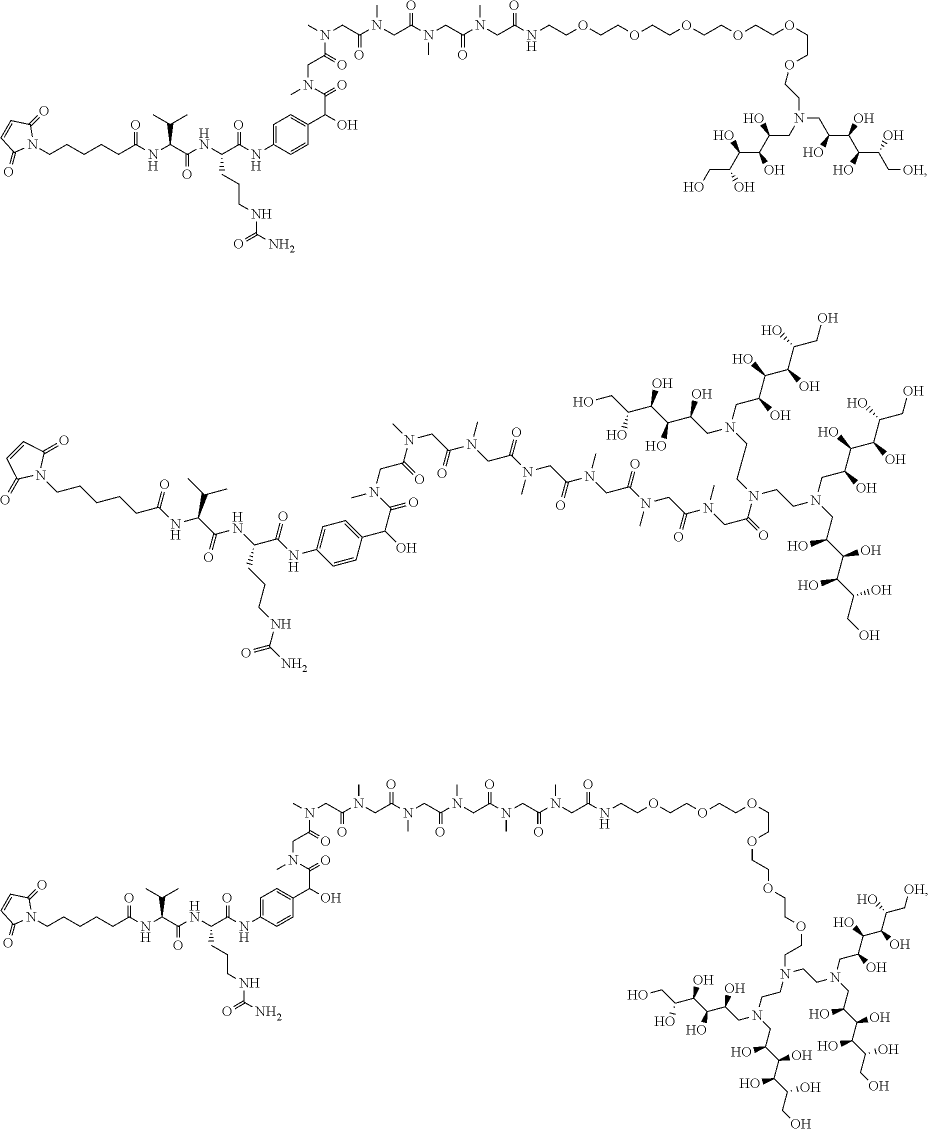

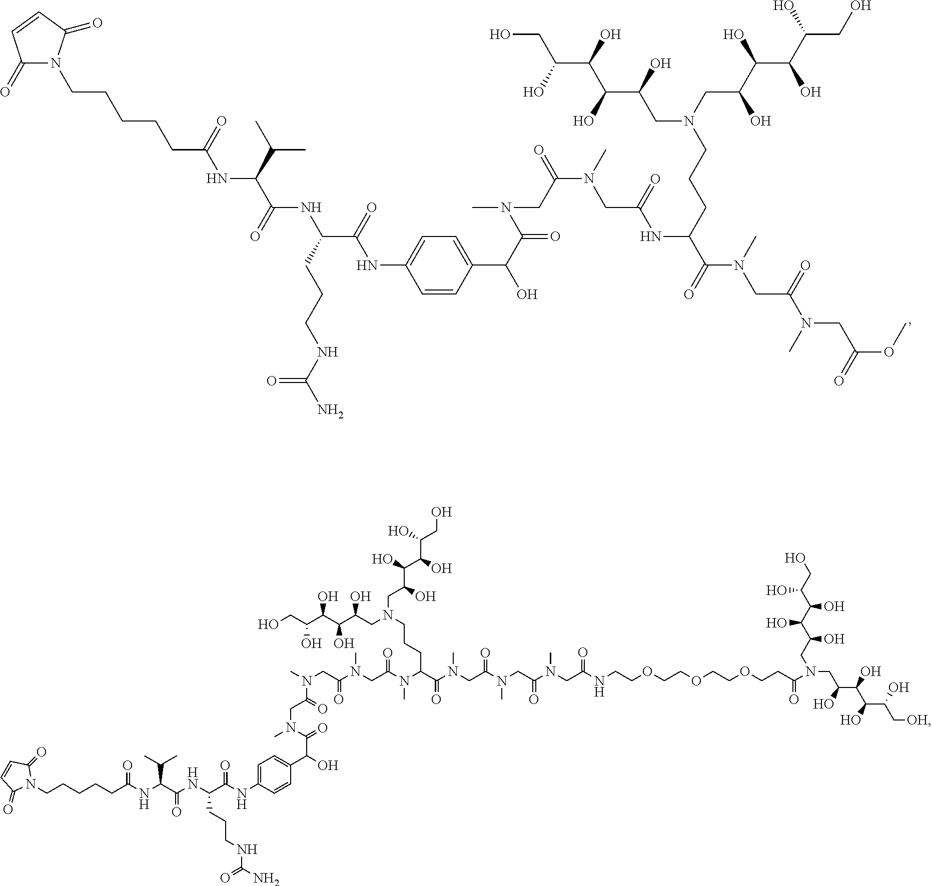

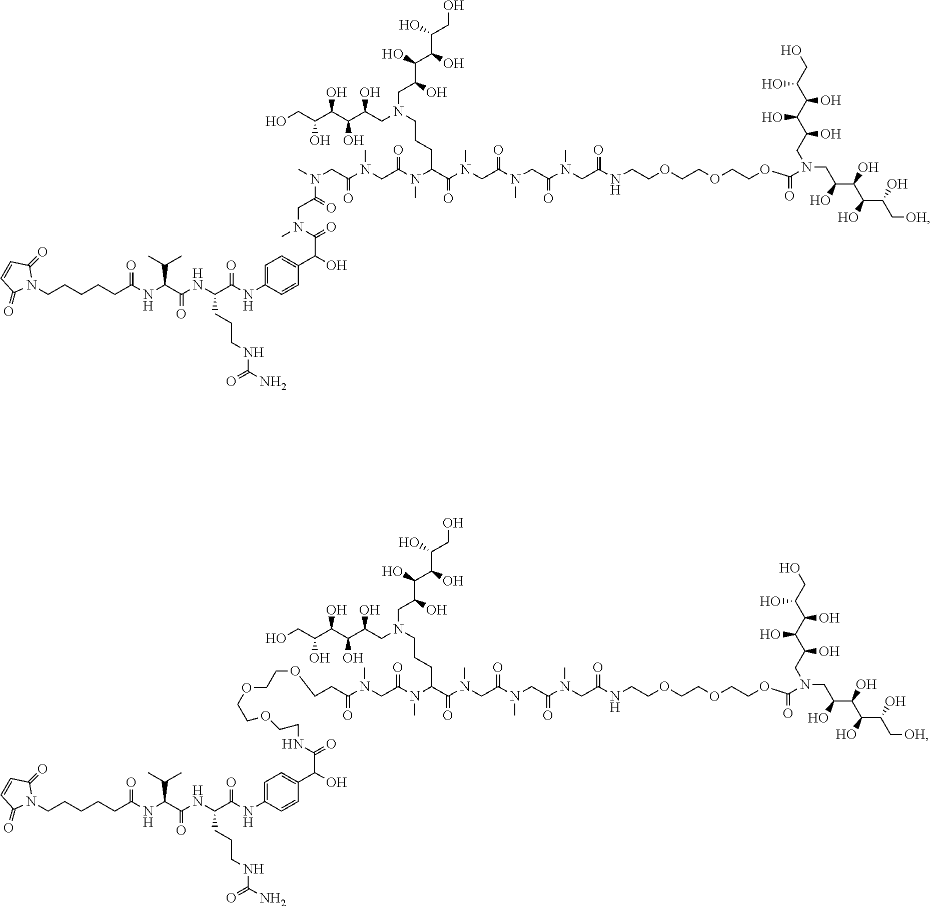

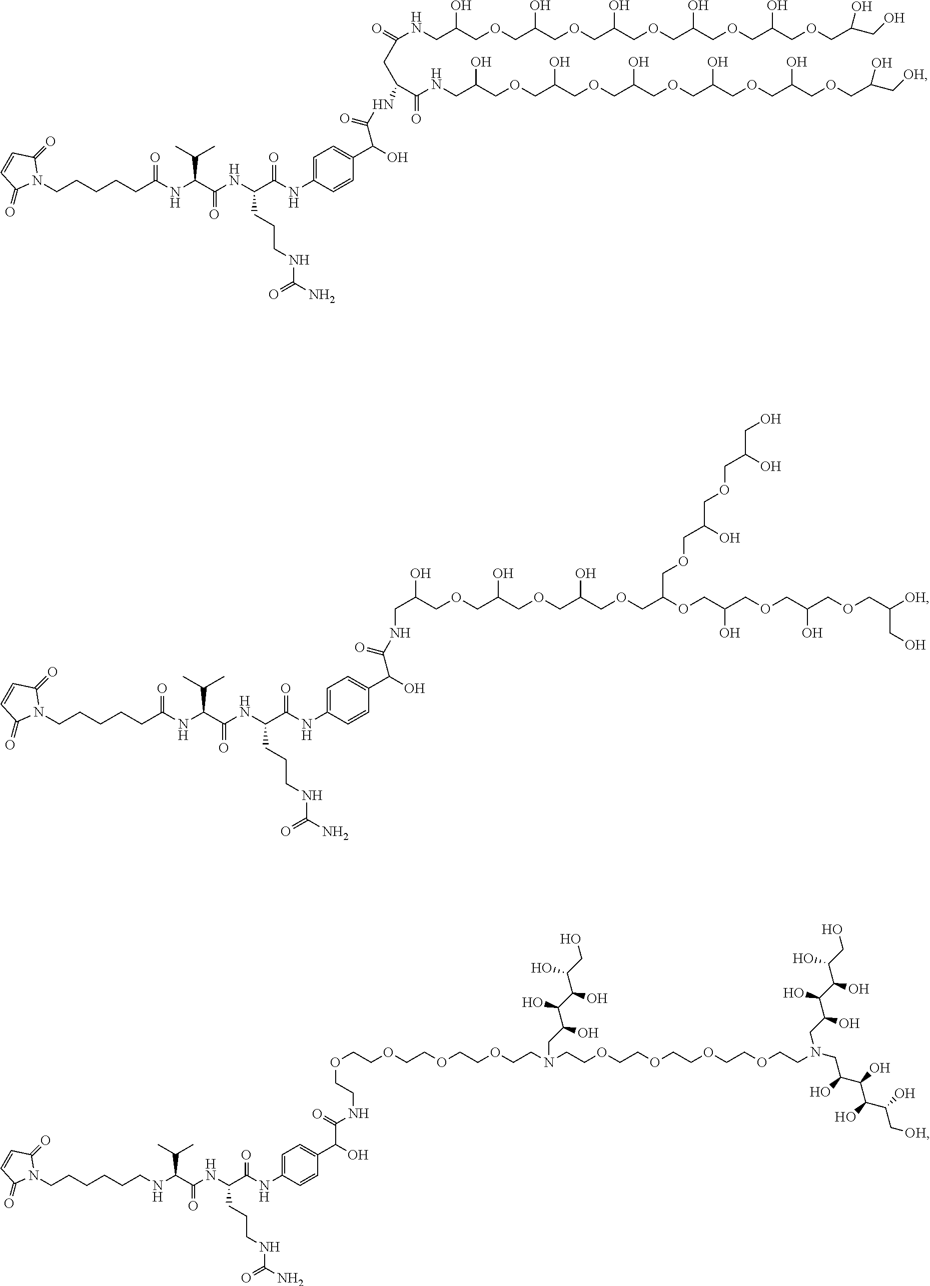

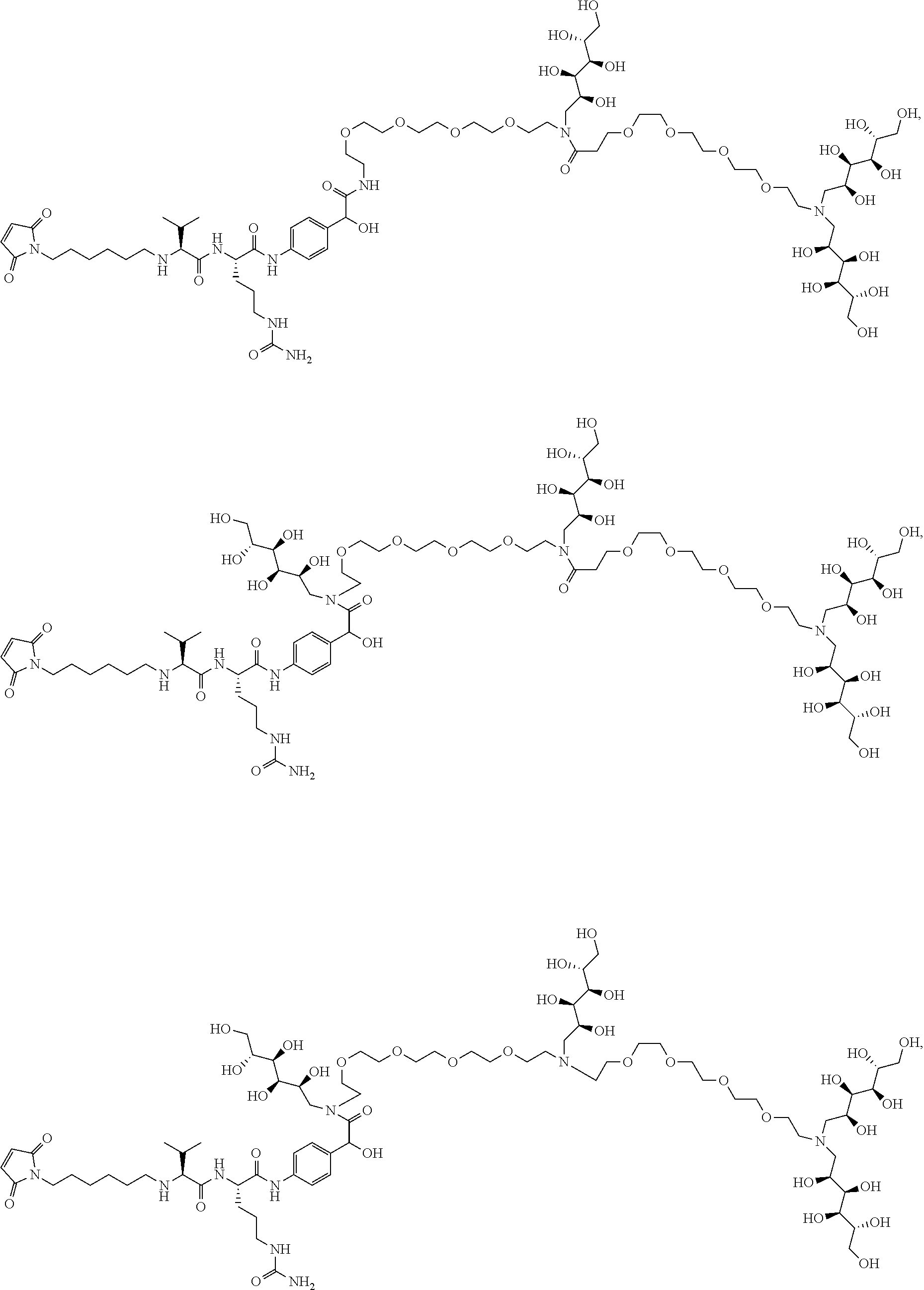

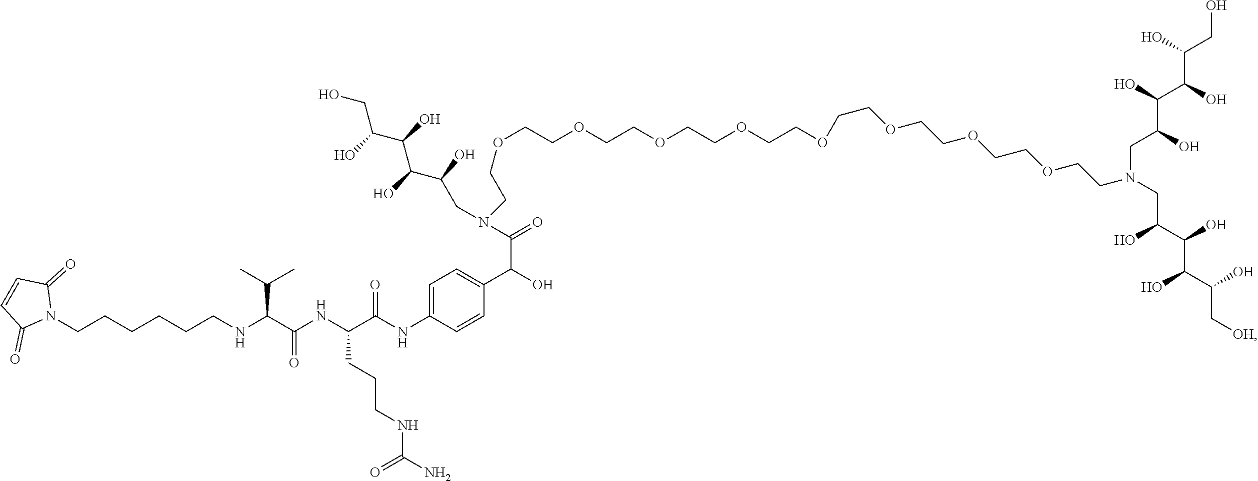 [SMILES: CC(C)[C@H](NCCCCCCN1C(=O)C=CC1=O)C(=O)N[C@@H](CCCNC(N)=O)C(=O)Nc1ccc(C(O)C(=O)N(CCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C[C@H](O)[C@@H](O)[C@H](O)[C@H](O)CO)cc1]

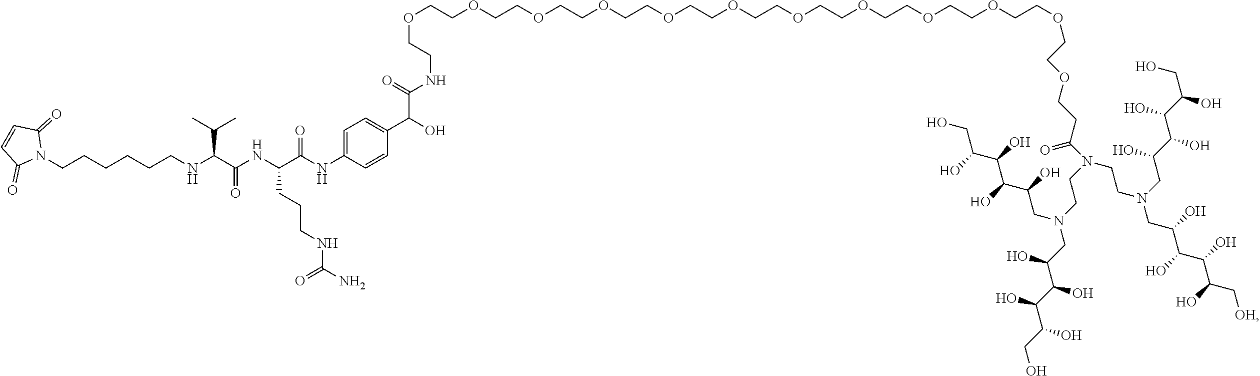 [SMILES: CC(C)[C@H](NCCCCCCN1C(=O)C=CC1=O)C(=O)N[C@@H](CCCNC(N)=O)C(=O)Nc1ccc(C(O)C(=O)NCCOCCOCCOCCOCCOCCOCCOCCOCCOCCOCCOCCOCCC(=O)N(CCN(C[C@H](O)[C@@H](O)[C@H](O)[C@H](O)CO)C[C@H](O)[C@@H](O)[C@H](O)[C@H](O)CO)CCN(C[C@H](O)[C@@H](O)[C@H](O)[C@H](O)CO)C[C@H](O)[C@@H](O)[C@H](O)[C@H](O)CO)cc1]

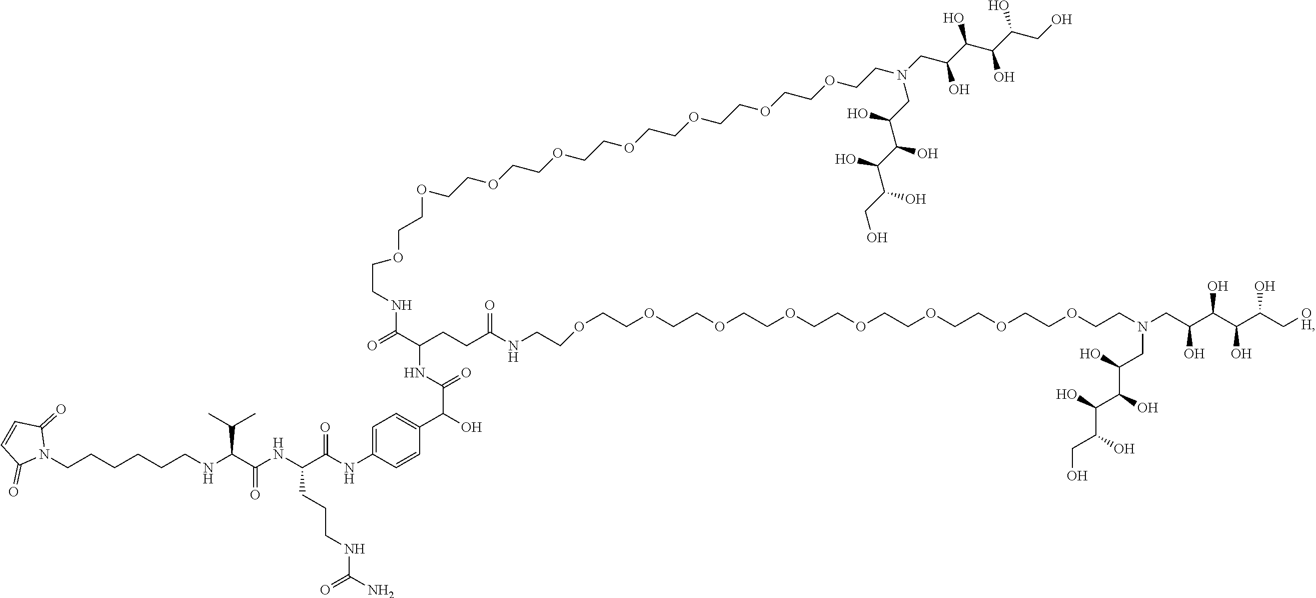 [SMILES: CC(C)[C@H](NCCCCCCN1C(=O)C=CC1=O)C(=O)N[C@@H](CCCNC(N)=O)C(=O)Nc1ccc(C(O)C(=O)NC(CCC(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

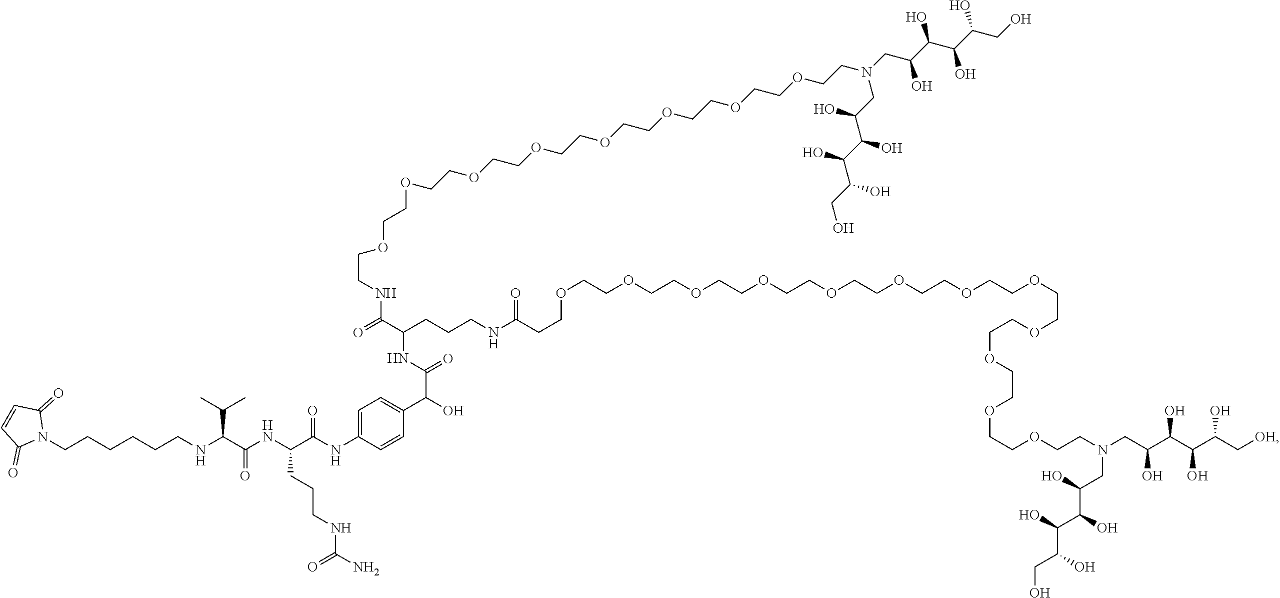 [SMILES: CC(C)[C@H](NCCCCCCN1C(=O)C=CC1=O)C(=O)N[C@@H](CCCNC(N)=O)C(=O)Nc1ccc(C(O)C(=O)NC(CCCNC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

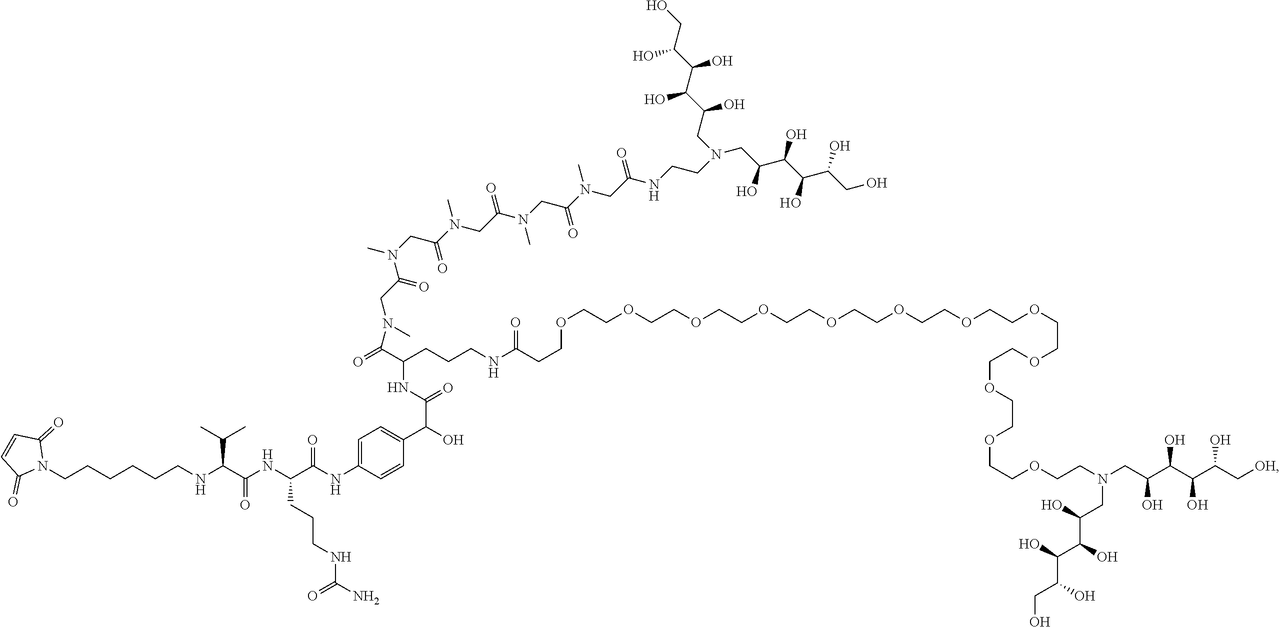 [SMILES: CC(C)[C@H](NCCCCCCN1C(=O)C=CC1=O)C(=O)N[C@@H](CCCNC(N)=O)C(=O)Nc1ccc(C(O)C(=O)NC(CCCNC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)cc1]

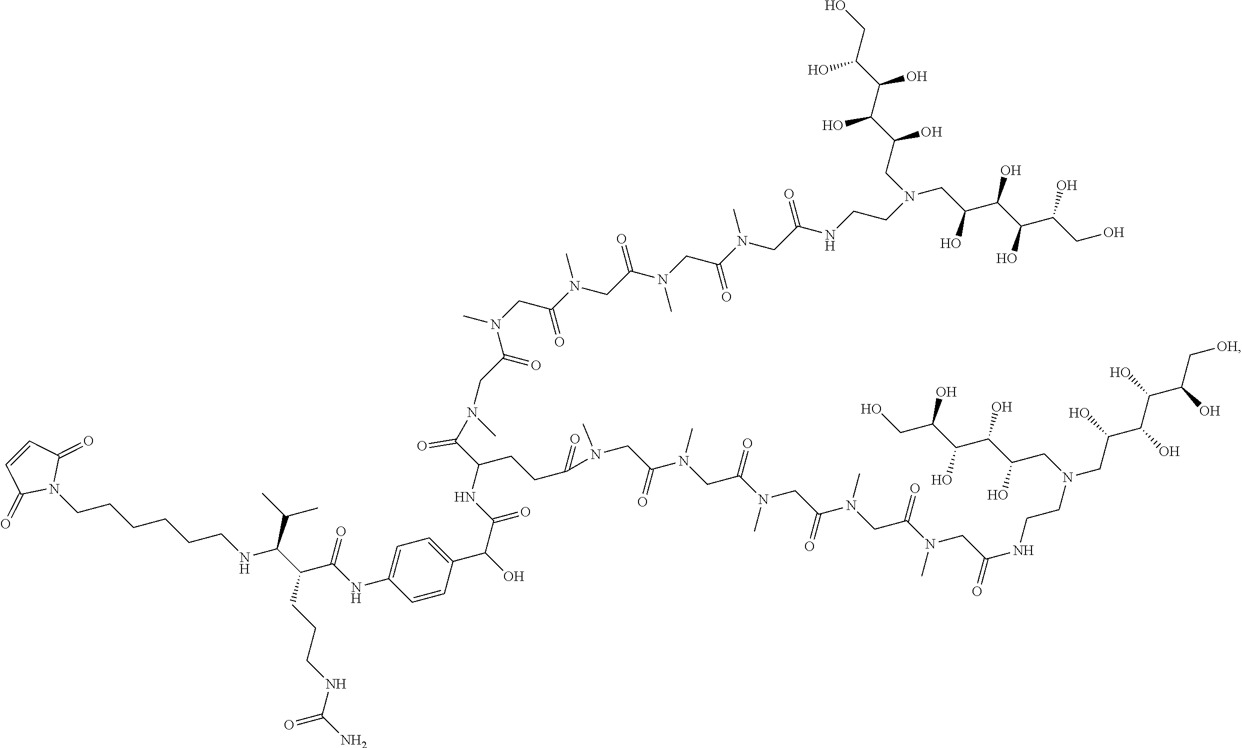 [SMILES: CC(C)[C@H](NCCCCCCN1C(=O)C=CC1=O)[C@@H](CCCNC(N)=O)C(=O)Nc1ccc(C(O)C(=O)NC(CCC(=O)N(C)CC(=O)N(C)CC(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)cc1]

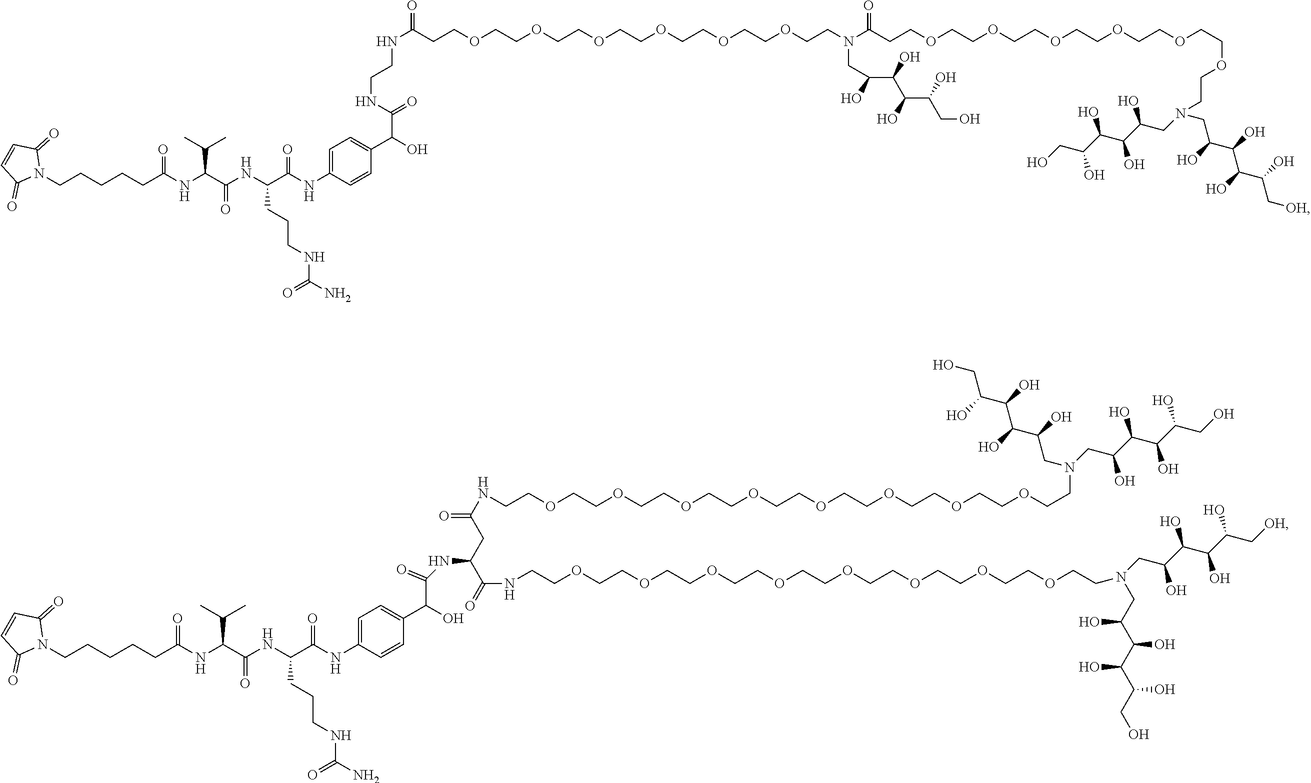

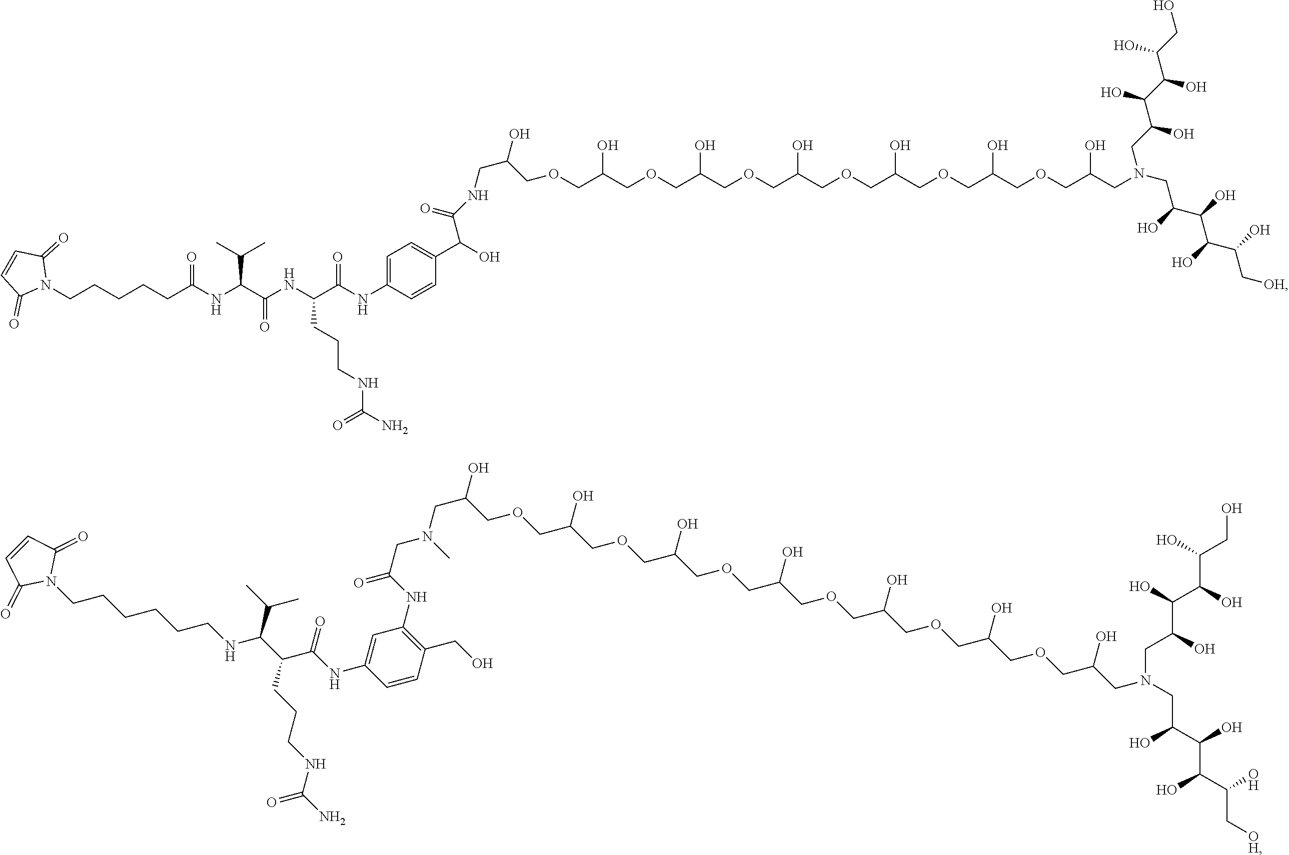 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCC(O)COCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)cc1 ; CC(C)[C@H](NCCCCCCN1C(=O)C=CC1=O)[C@@H](CCCNC(N)=O)C(=O)Nc1ccc(CO)c(NC(=O)CN(C)CC(O)COCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)c1]

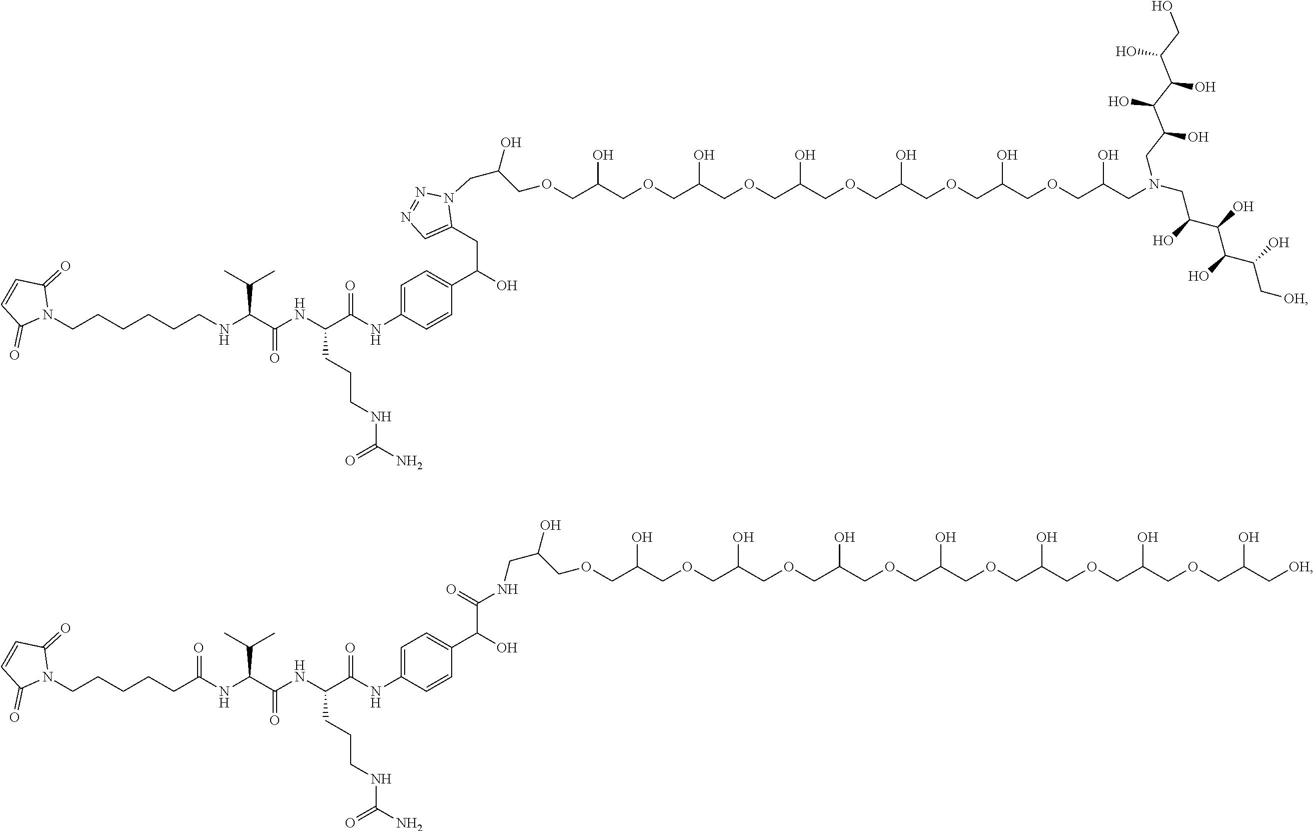 [SMILES: CC(C)[C@H](NCCCCCCN1C(=O)C=CC1=O)C(=O)N[C@@H](CCCNC(N)=O)C(=O)Nc1ccc(C(O)Cc2cnnn2CC(O)COCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)cc1 ; CC(C)[C@H](NC(=O)CCCCCN1C(=O)C=CC1=O)C(=O)N[C@@H](CCCNC(N)=O)C(=O)Nc1ccc(C(O)C(=O)NCC(O)COCC(O)COCC(O)COCC(O)COCC(O)COCC(O)COCC(O)COCC(O)CO)cc1]

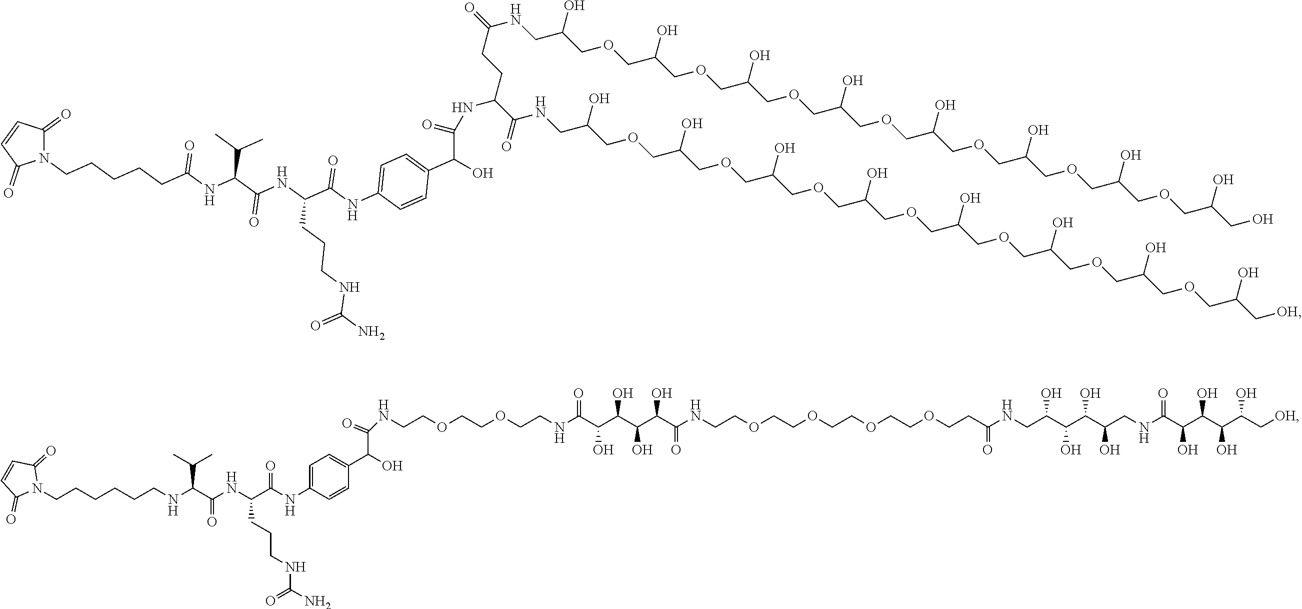 [SMILES: CC(C)[C@H](NC(=O)CCCCCN1C(=O)C=CC1=O)C(=O)N[C@@H](CCCNC(N)=O)C(=O)Nc1ccc(C(O)C(=O)NC(CCC(=O)NCC(O)COCC(O)COCC(O)COCC(O)COCC(O)COCC(O)COCC(O)COCC(O)CO)C(=O)NCC(O)COCC(O)COCC(O)COCC(O)COCC(O)COCC(O)COCC(O)COCC(O)CO)cc1 ; CC(C)[C@H](NCCCCCCN1C(=O)C=CC1=O)C(=O)N[C@@H](CCCNC(N)=O)C(=O)Nc1ccc(C(O)C(=O)NCCOCCOCCNC(=O)[C@@H](O)[C@@H](O)[C@H](O)[C@@H](O)C(=O)NCCOCCOCCOCCOCCC(=O)NC[C@H](O)[C@@H](O)[C@H](O)[C@H](O)CNC(=O)[C@H](O)[C@@H](O)[C@H](O)[C@H](O)CO)cc1]

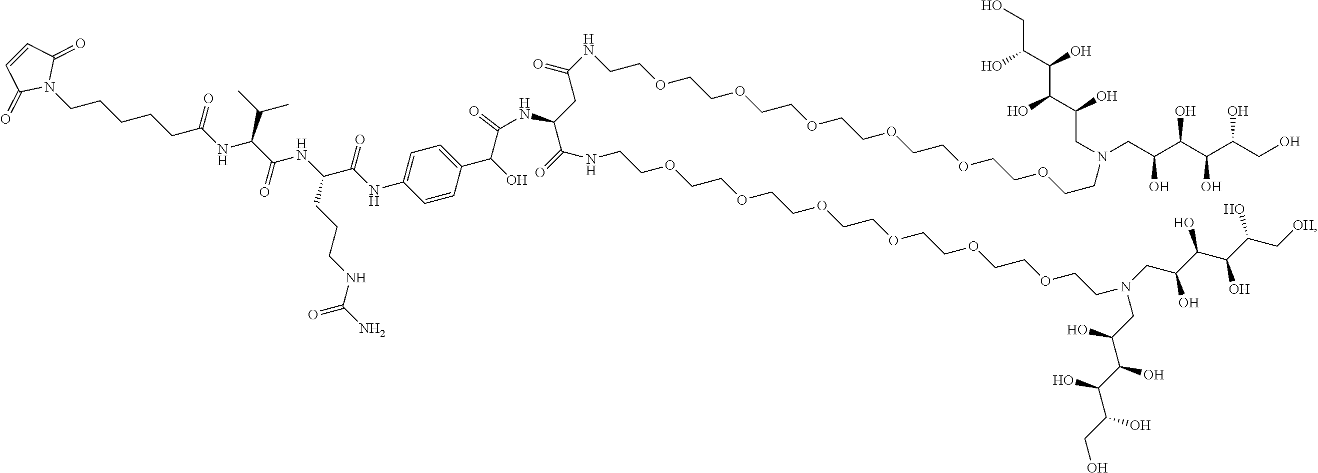 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@@H](CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

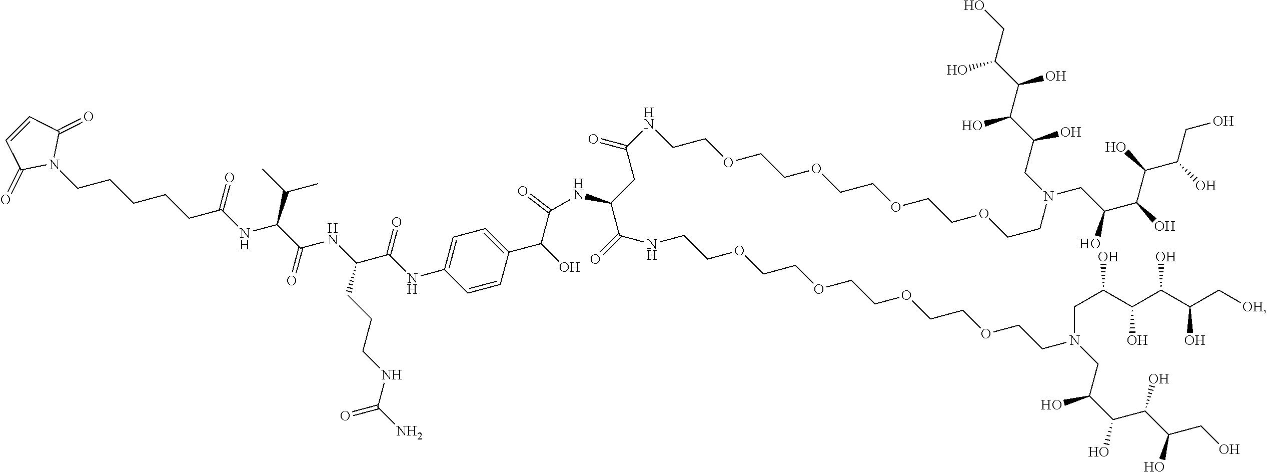 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@@H](CC(=O)NCCOCCOCCOCCOCCN(C[C@H](O)[C@H](O)[C@@H](O)[C@@H](O)CO)C[C@H](O)[C@@H](O)[C@H](O)[C@H](O)CO)C(=O)NCCOCCOCCOCCOCCN(C[C@H](O)[C@@H](O)[C@H](O)[C@H](O)CO)C[C@H](O)[C@@H](O)[C@H](O)[C@H](O)CO)cc1]

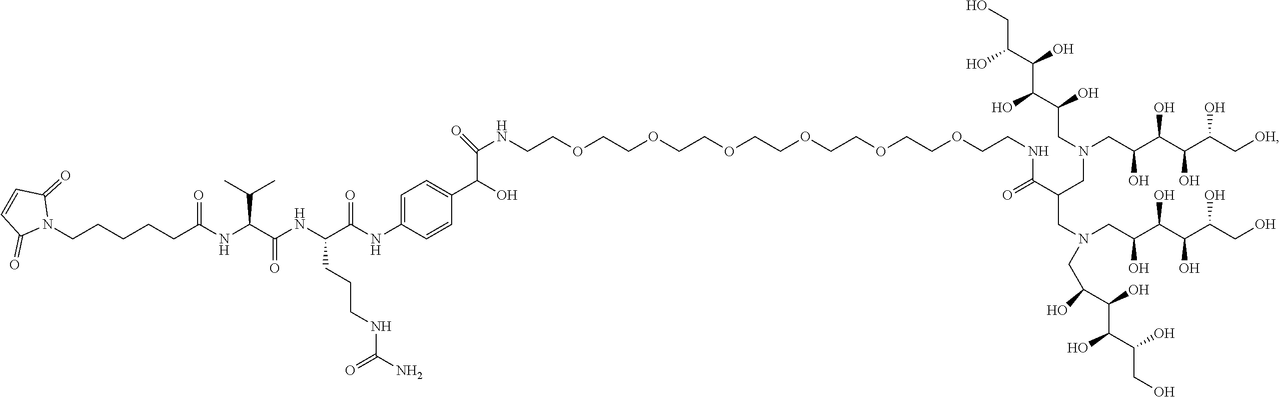 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCCOCCOCCOCCOCCOCCOCCNC(=O)C(CN(C[C@H](O)[C@@H](O)[C@H](O)[C@H](O)CO)C[C@H](O)[C@@H](O)[C@H](O)[C@H](O)CO)CN(C[C@H](O)[C@@H](O)[C@H](O)[C@H](O)CO)C[C@H](O)[C@@H](O)[C@H](O)[C@H](O)CO)cc1]

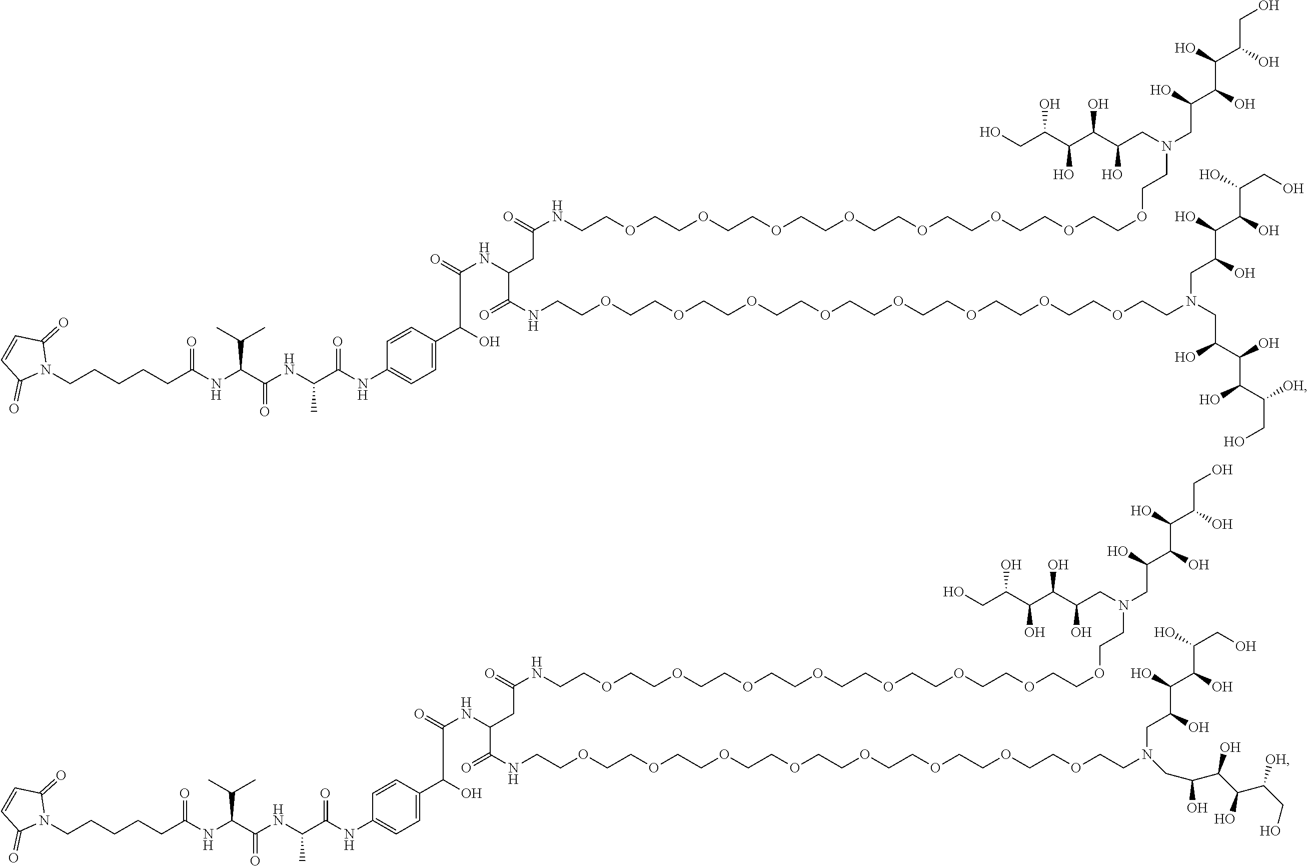

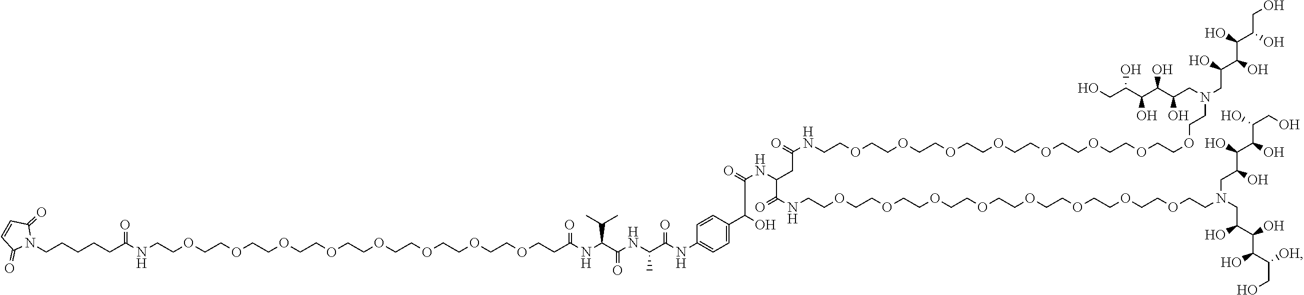 [SMILES: CC(C)[C@H](NC(=O)CCOCCOCCOCCOCCOCCOCCOCCOCCNC(=O)CCCCCN1C(=O)C=CC1=O)C(=O)N[C@@H](C)C(=O)Nc1ccc(C(O)C(=O)NC(CC(=O)NCCOCCOCCOCCOCCOCCOCCOCCOCCN(C[C@@H](O)[C@H](O)[C@@H](O)[C@@H](O)CO)C[C@@H](O)[C@H](O)[C@@H](O)[C@@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

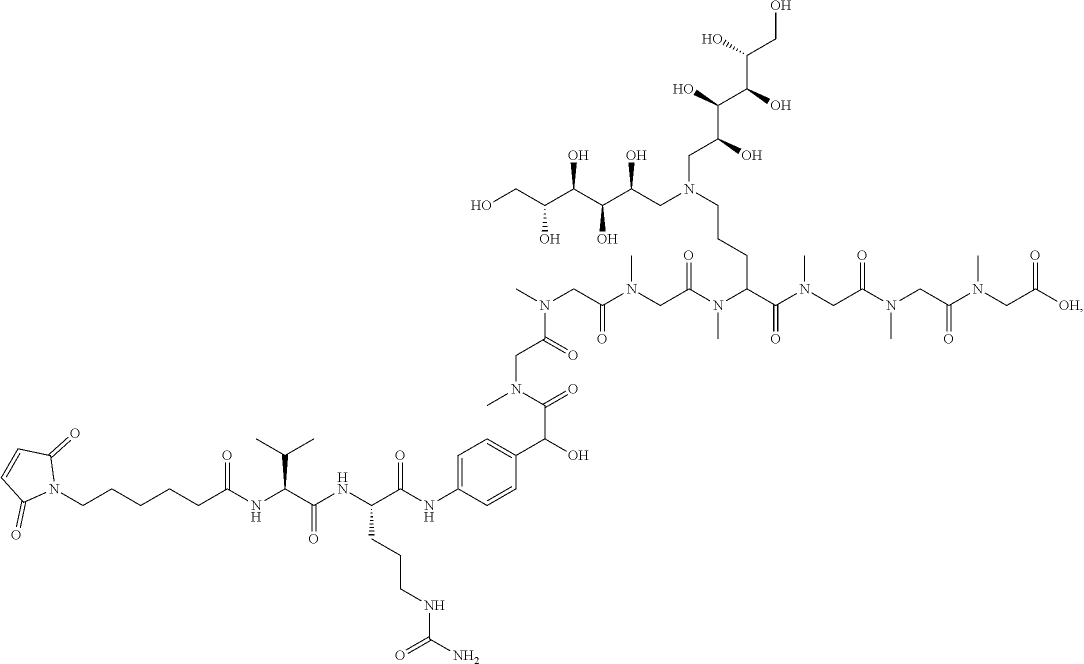 [SMILES: CC(C)[C@H](NC(=O)CCCCCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)O)cc1]

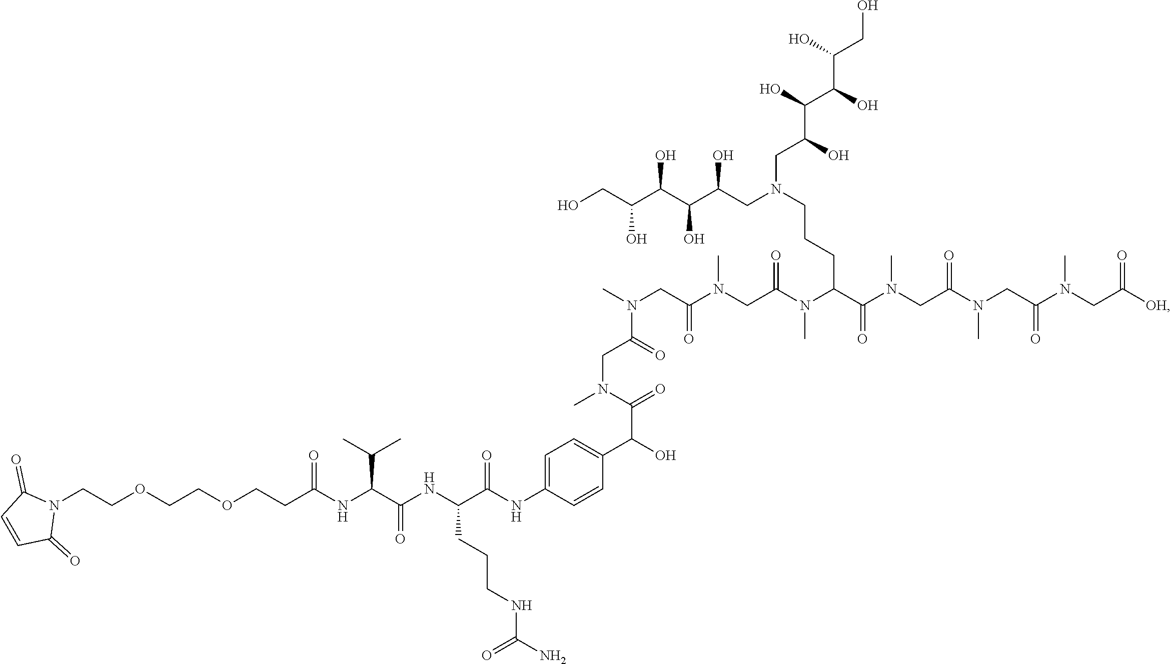 [SMILES: CC(C)[C@H](NC(=O)CCOCCOCCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)O)cc1]

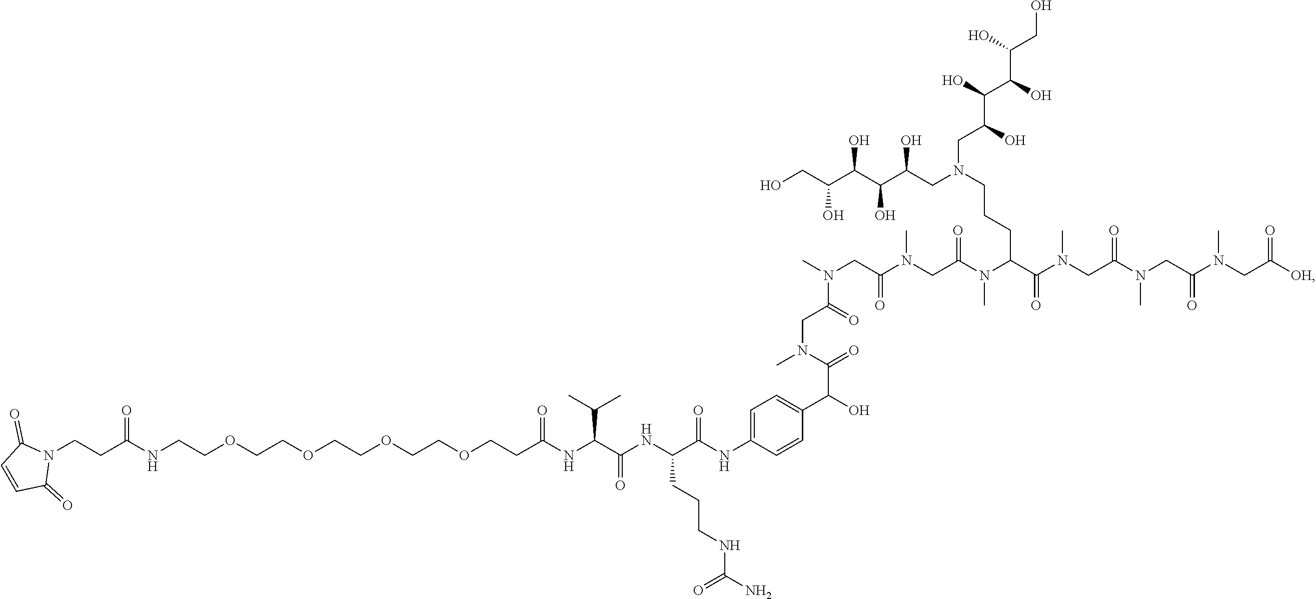 [SMILES: CC(C)[C@H](NC(=O)CCOCCOCCOCCOCCNC(=O)CCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)O)cc1]

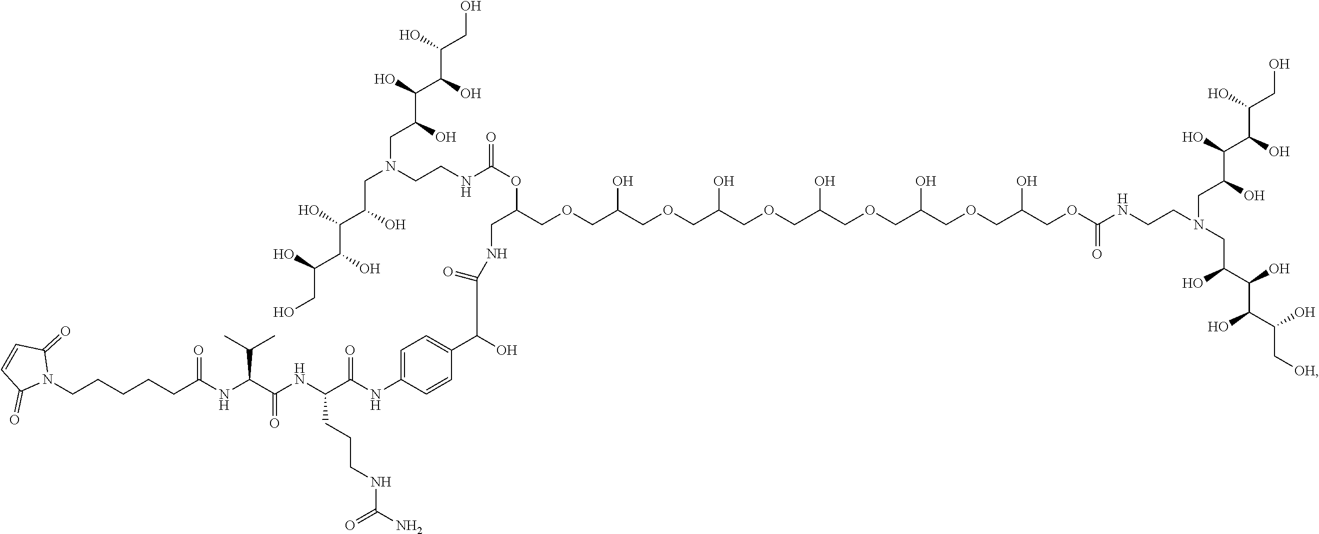 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCC(COCC(O)COCC(O)COCC(O)COCC(O)COCC(O)COC(=O)NCCN(C[C@H](O)[C@@H](O)[C@H](O)[C@H](O)CO)C[C@H](O)[C@@H](O)[C@H](O)[C@H](O)CO)OC(=O)NCCN(C[C@H](O)[C@@H](O)[C@H](O)[C@H](O)CO)C[C@H](O)[C@@H](O)[C@H](O)[C@H](O)CO)cc1]

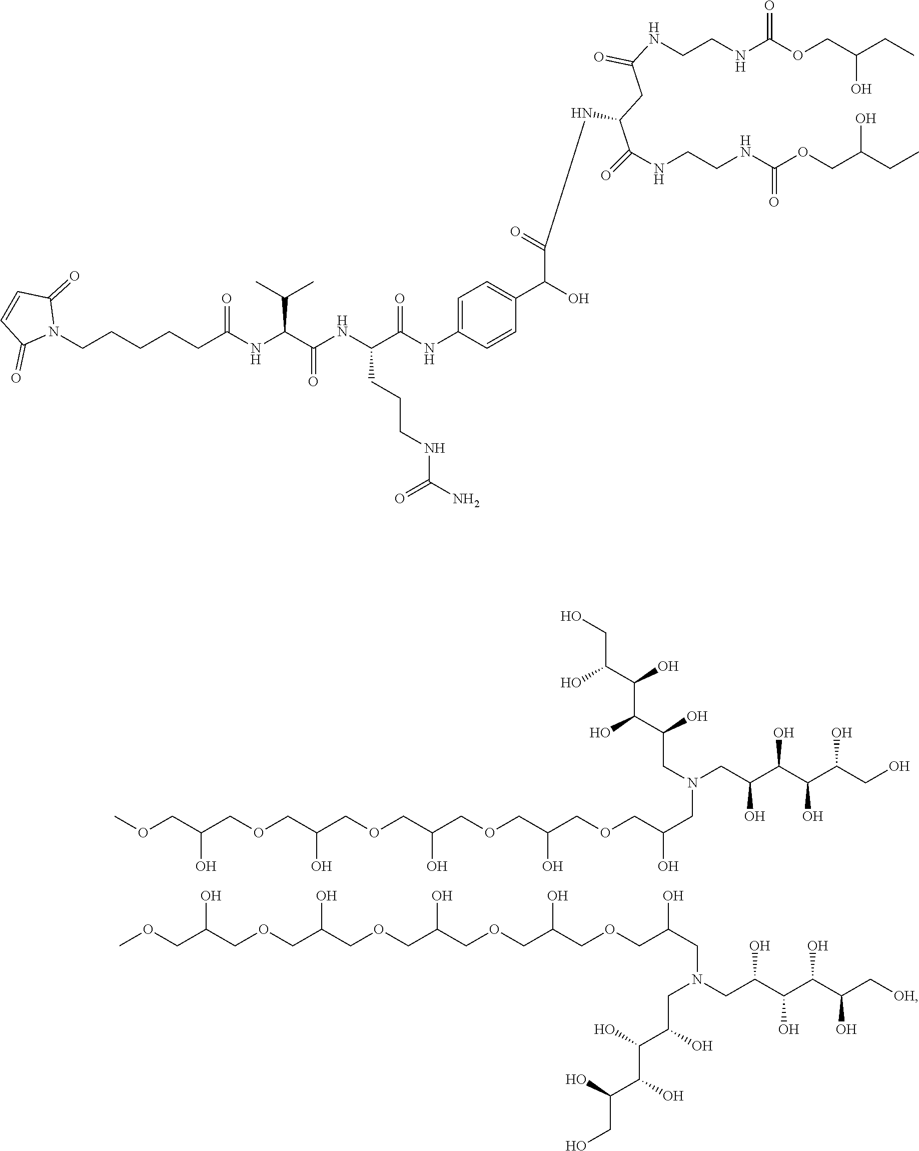

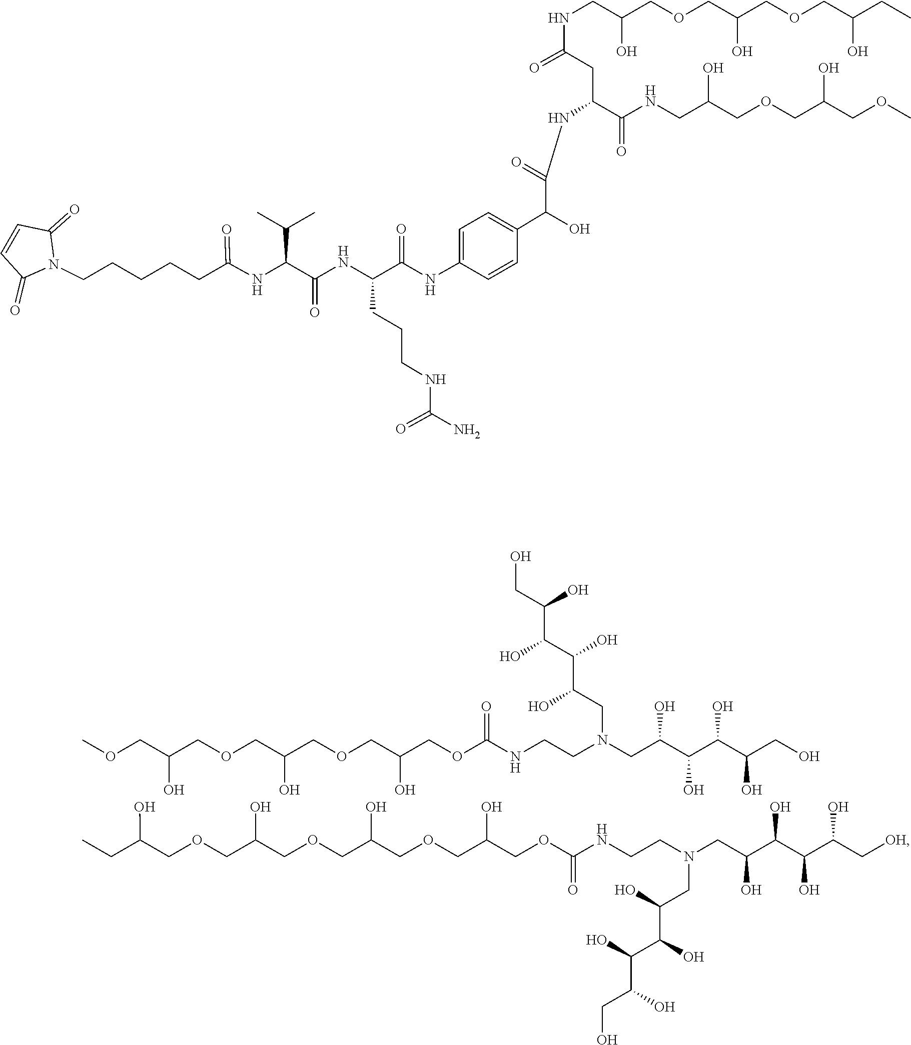

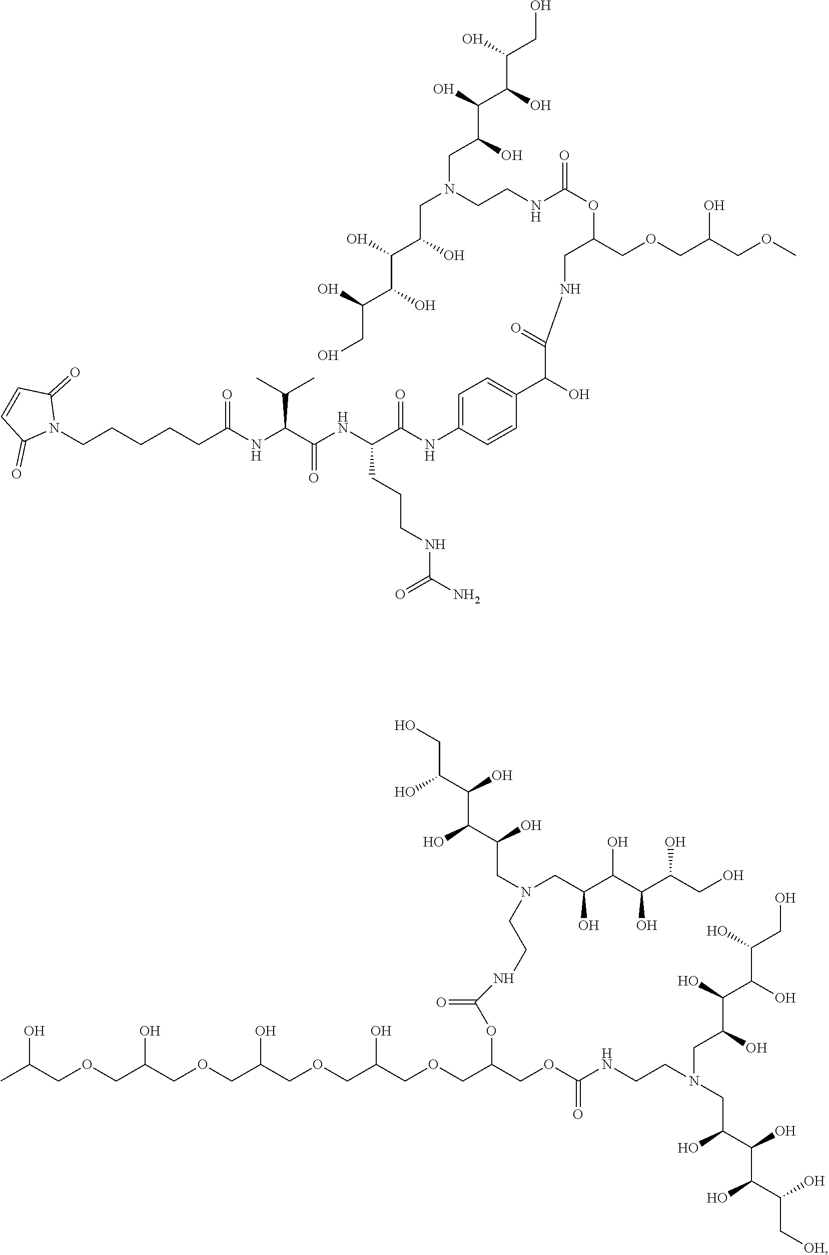

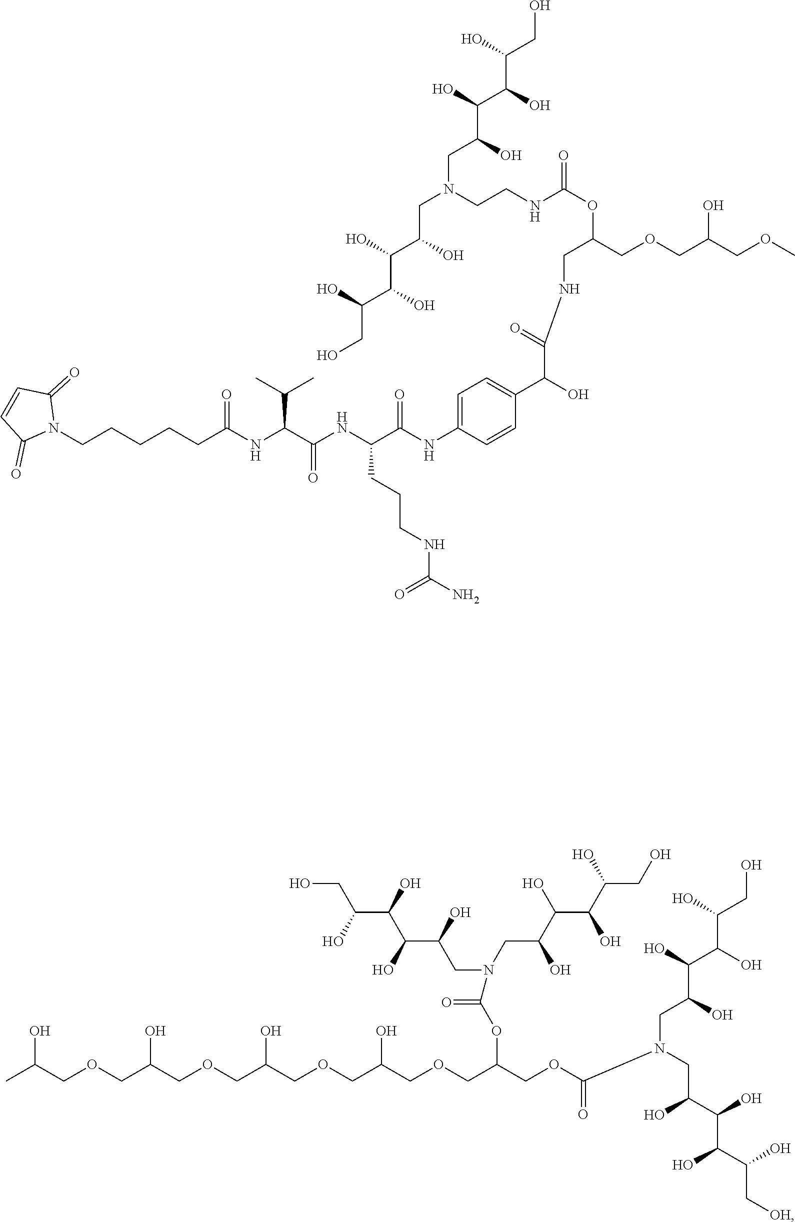

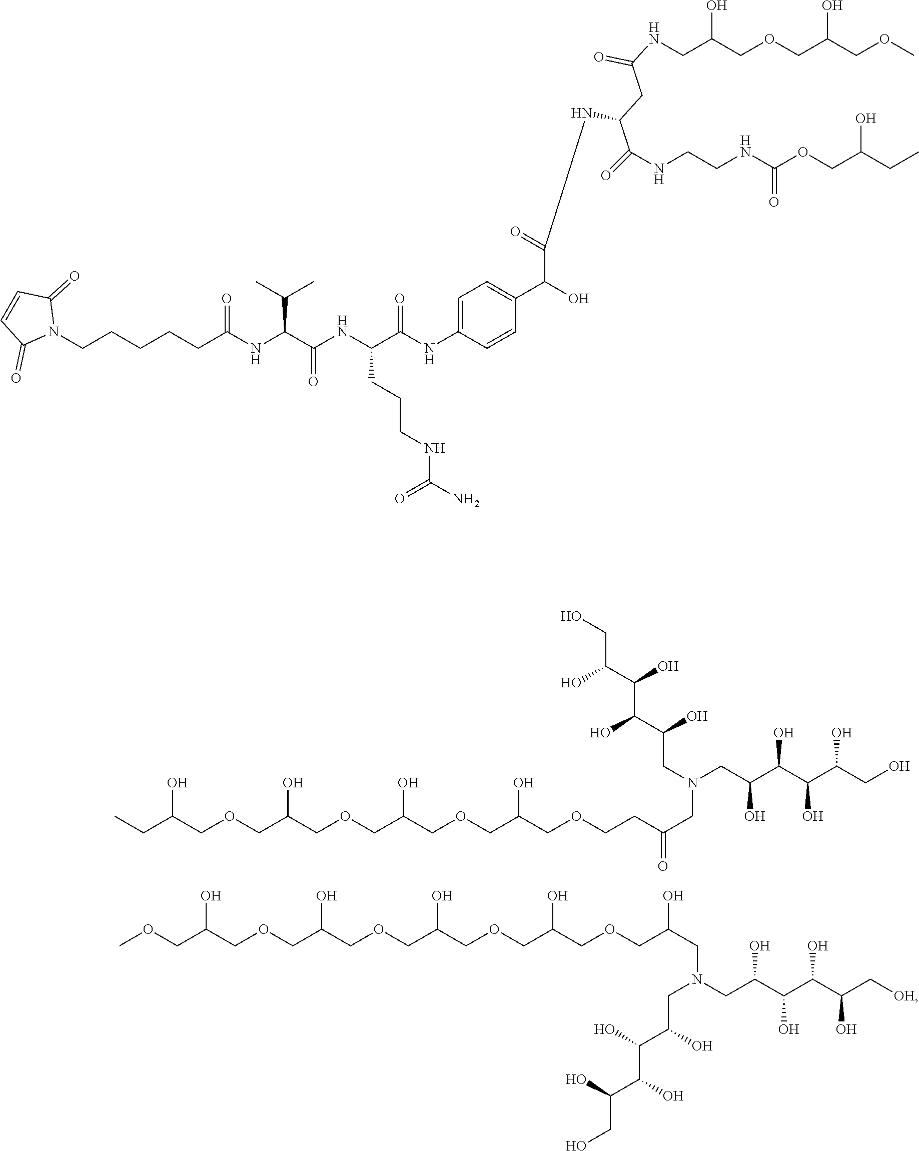

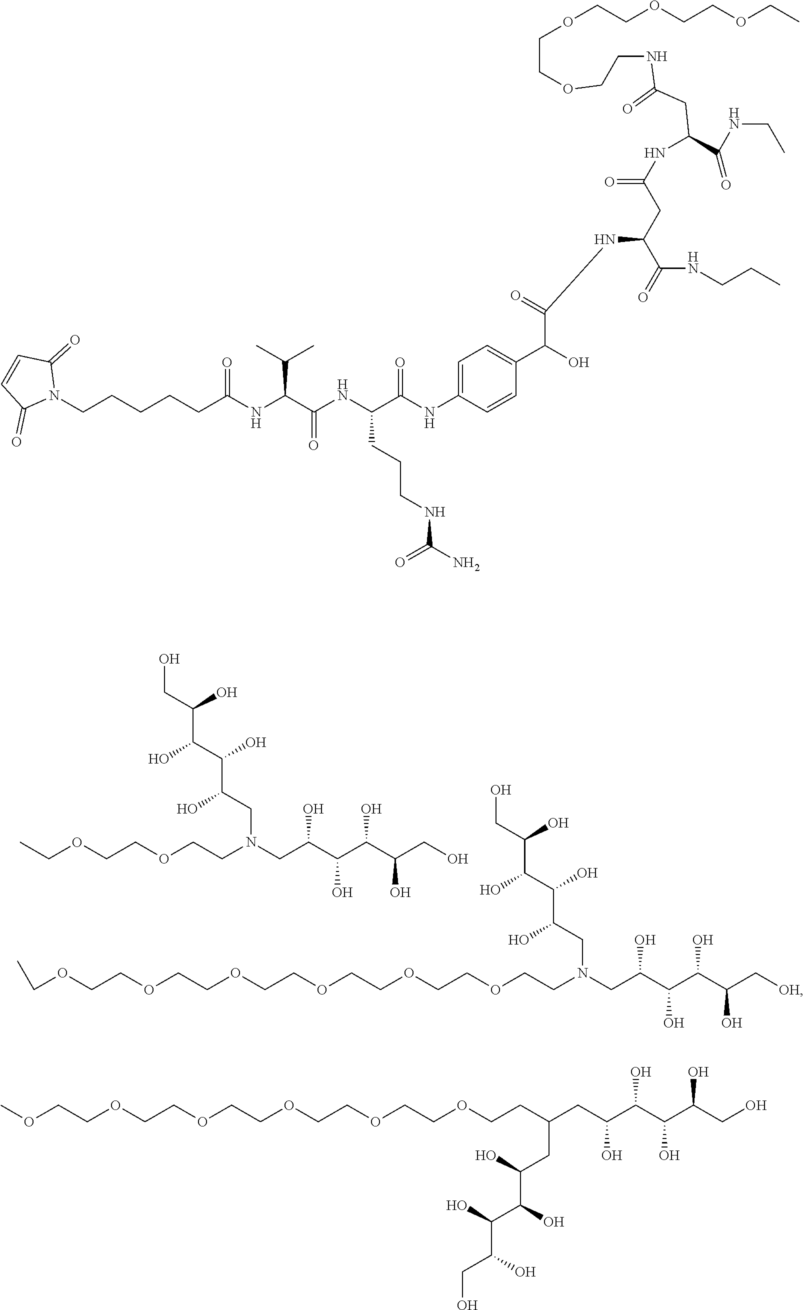

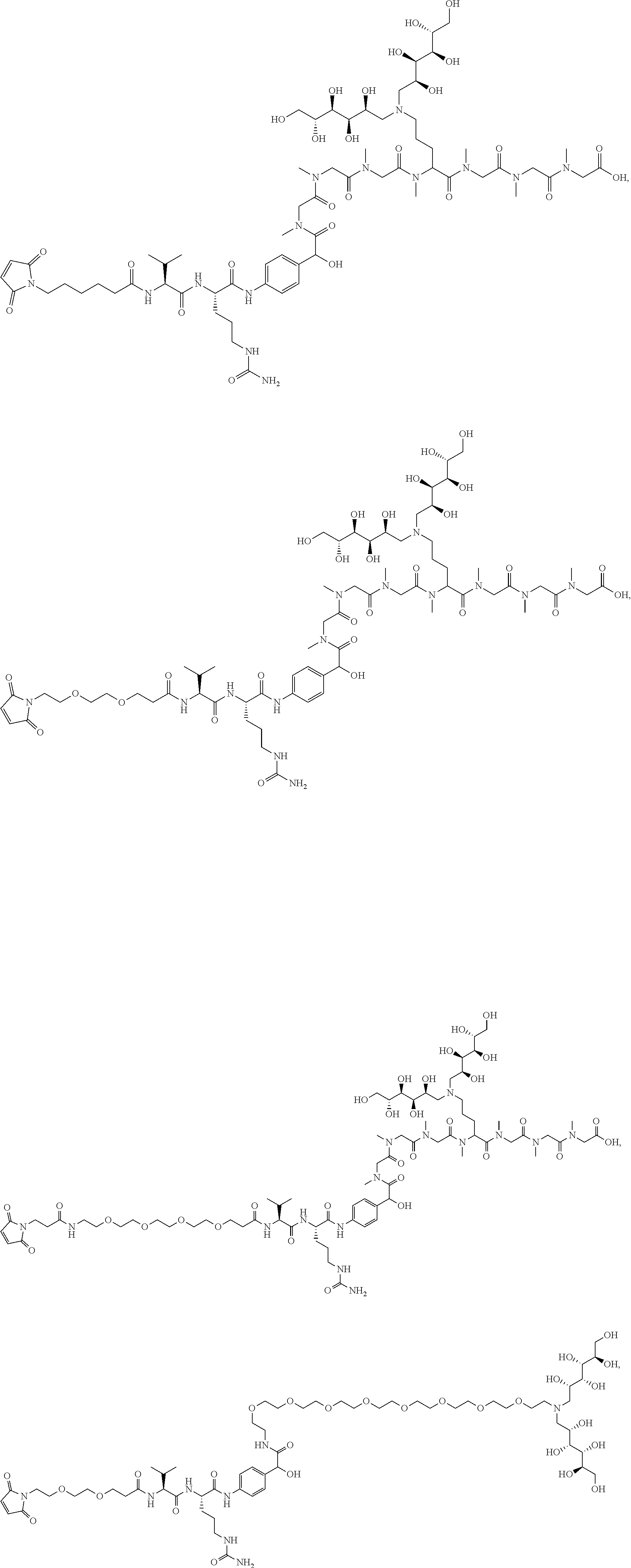

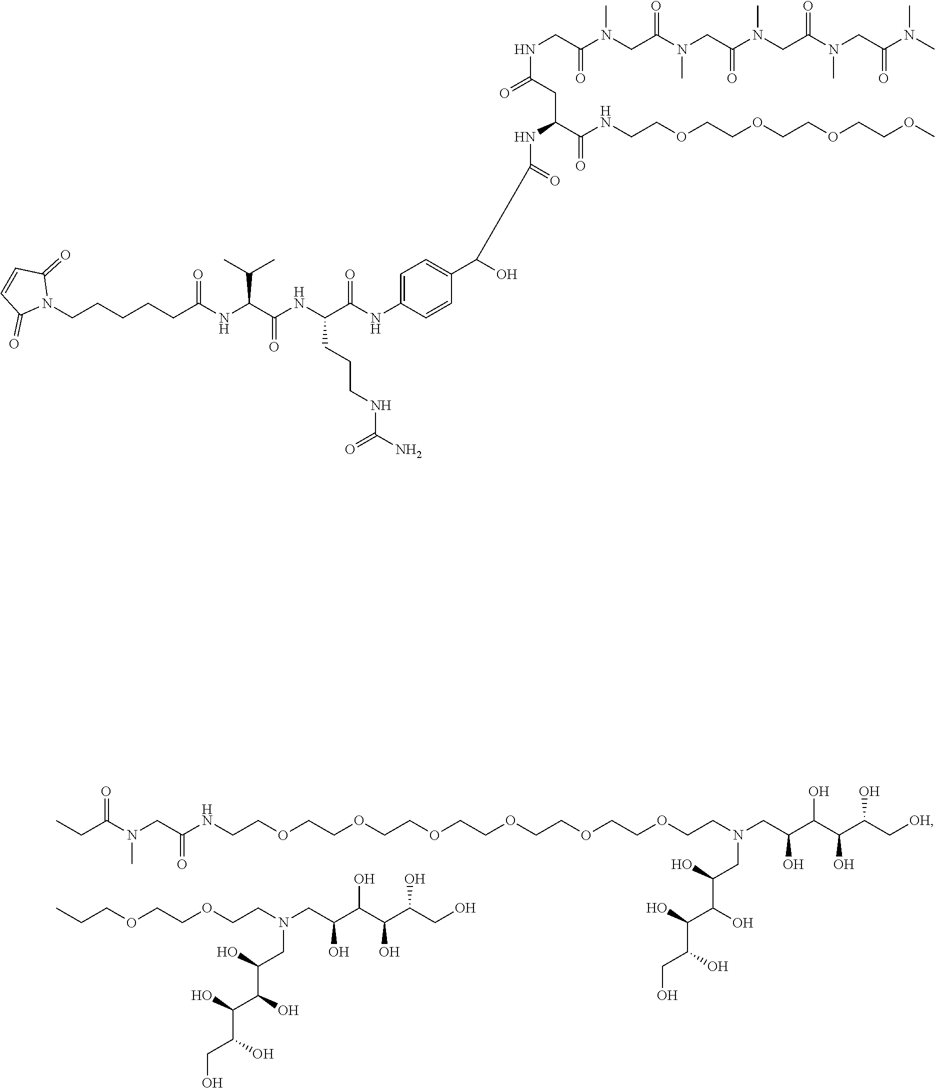

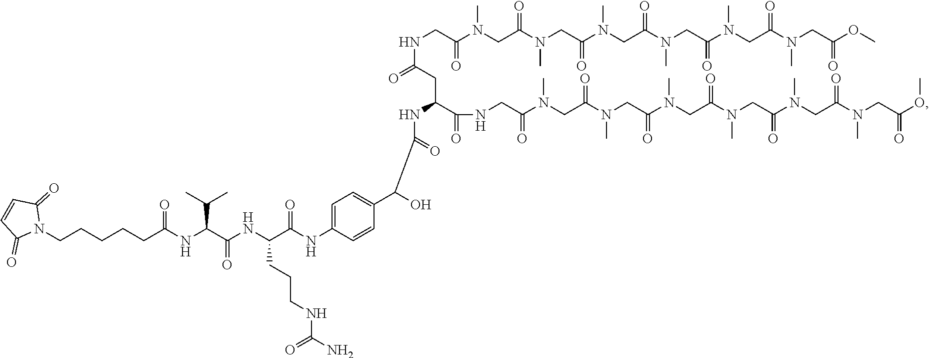 [SMILES: COC(=O)CN(C)C(=O)CN(C)C(=O)CN(C)C(=O)CN(C)C(=O)CN(C)C(=O)CN(C)C(=O)CNC(=O)C[C@H](NC(=O)C(O)c1ccc(NC(=O)[C@H](CCCNC(N)=O)NC(=O)[C@@H](NC(=O)CCCCCN2C(=O)C=CC2=O)C(C)C)cc1)C(=O)NCC(=O)N(C)CC(=O)N(C)CC(=O)N(C)CC(=O)N(C)CC(=O)N(C)CC(=O)N(C)CC(=O)OC]

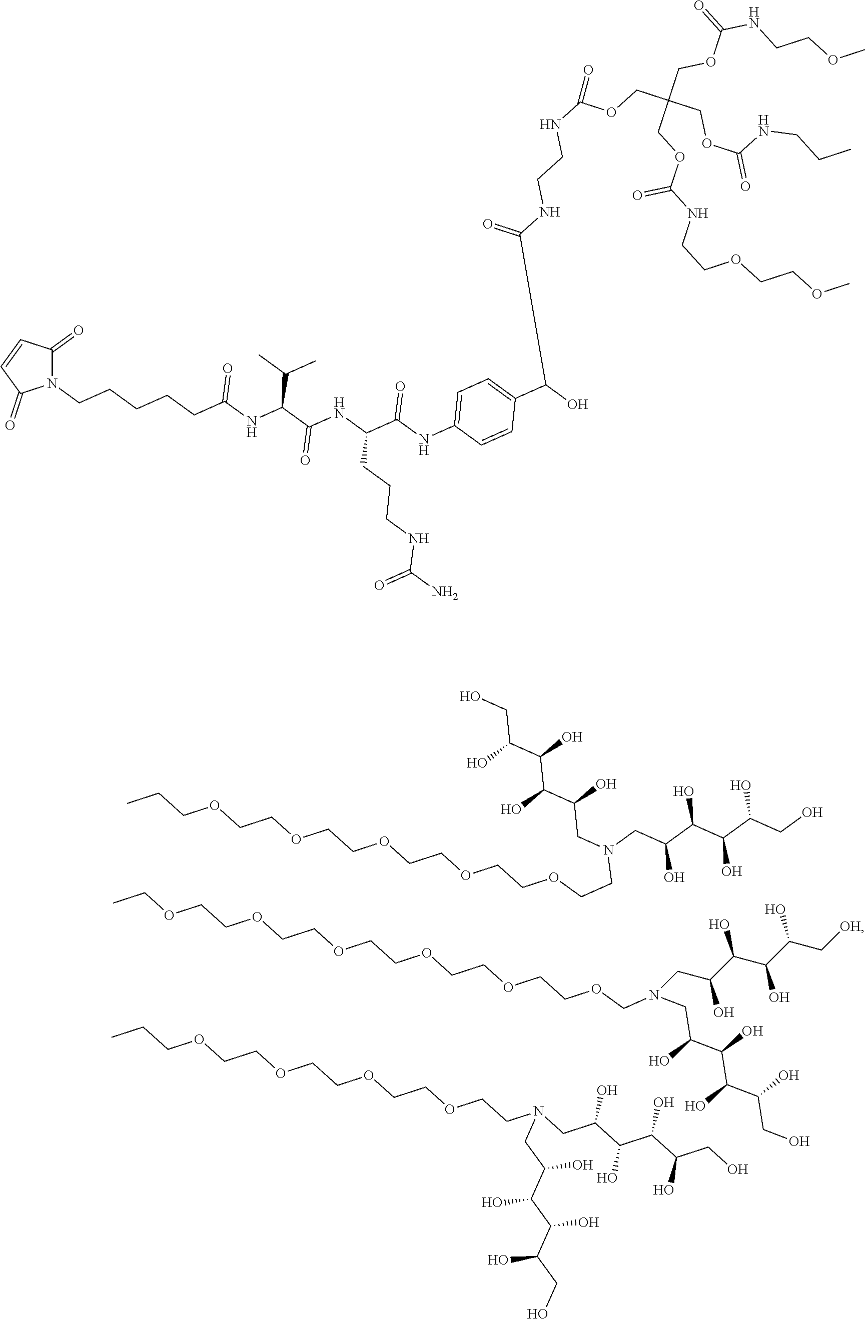

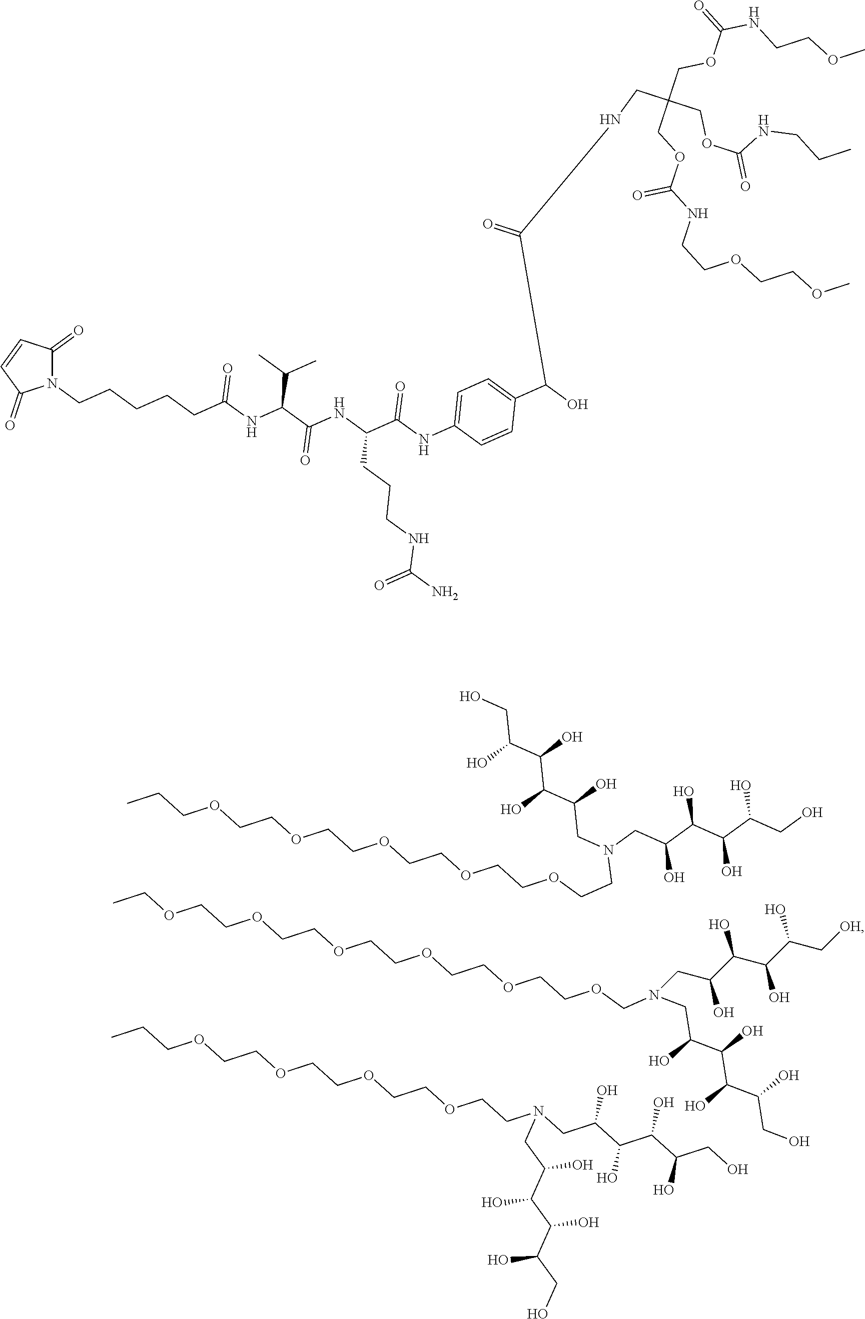

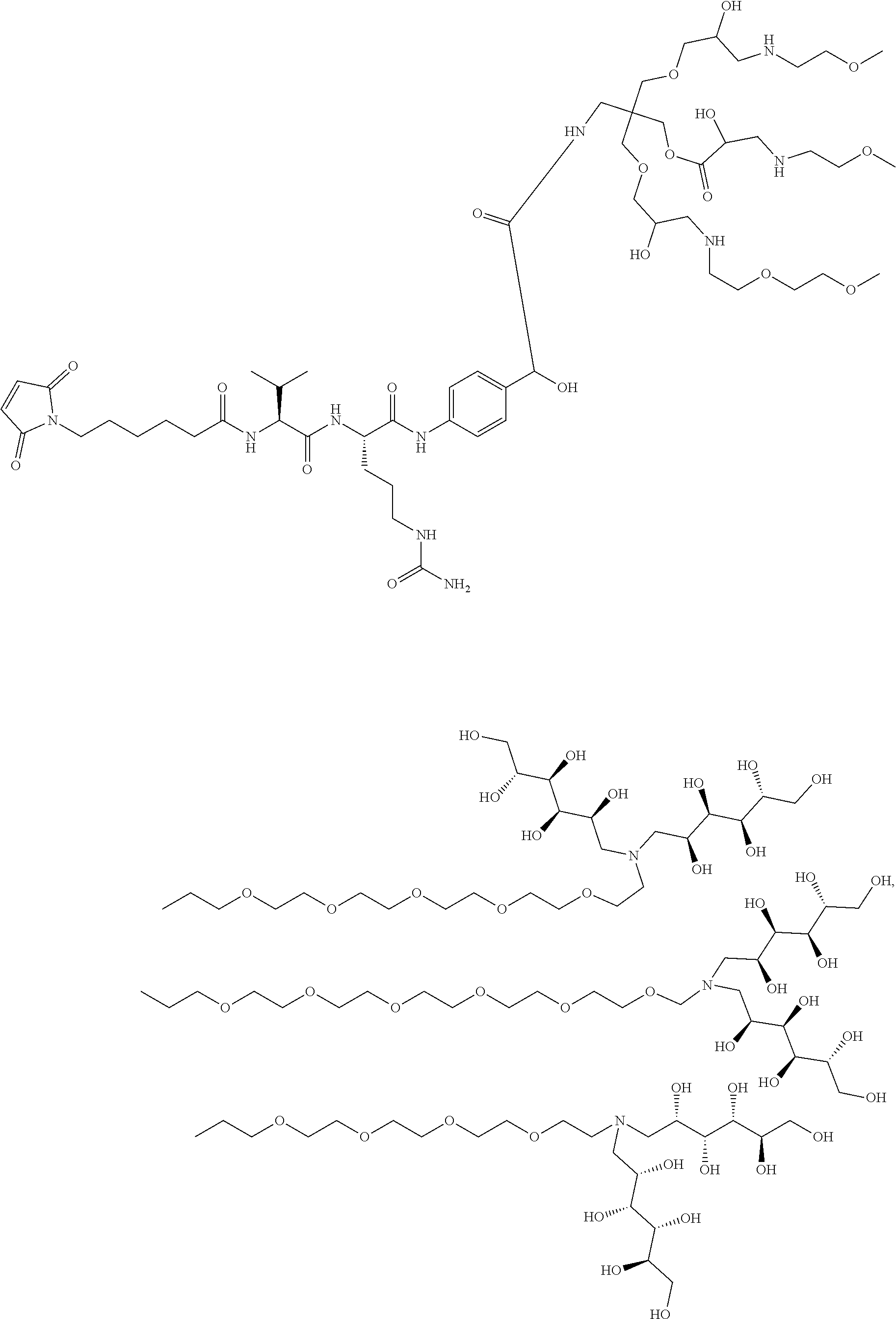

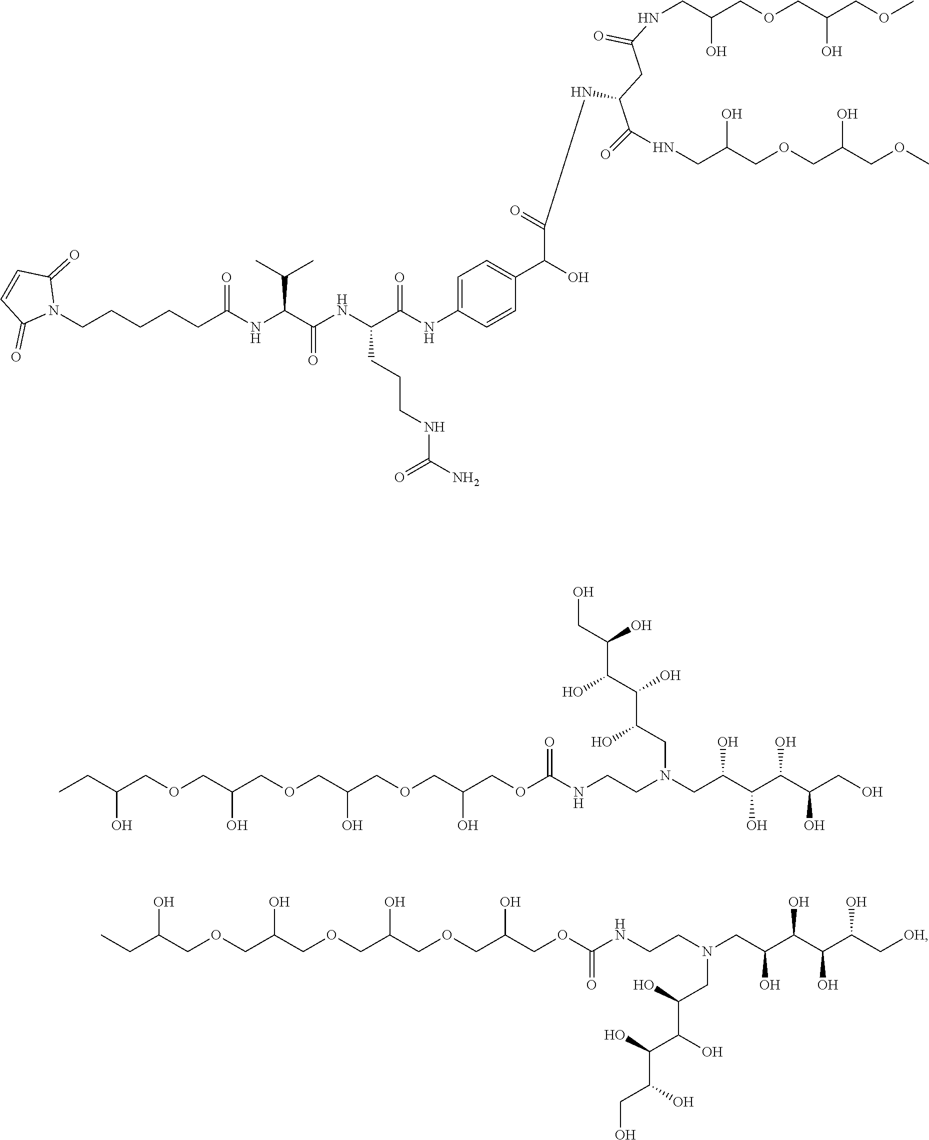

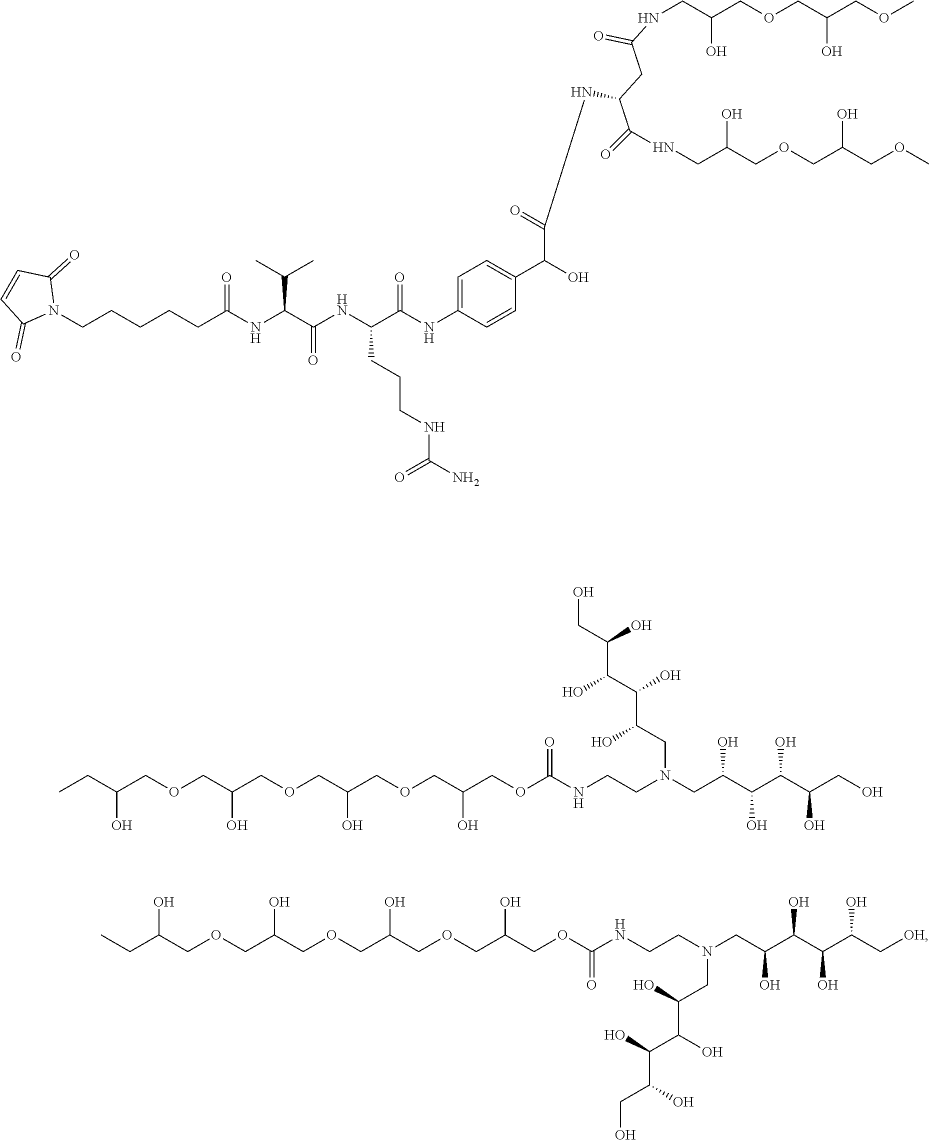

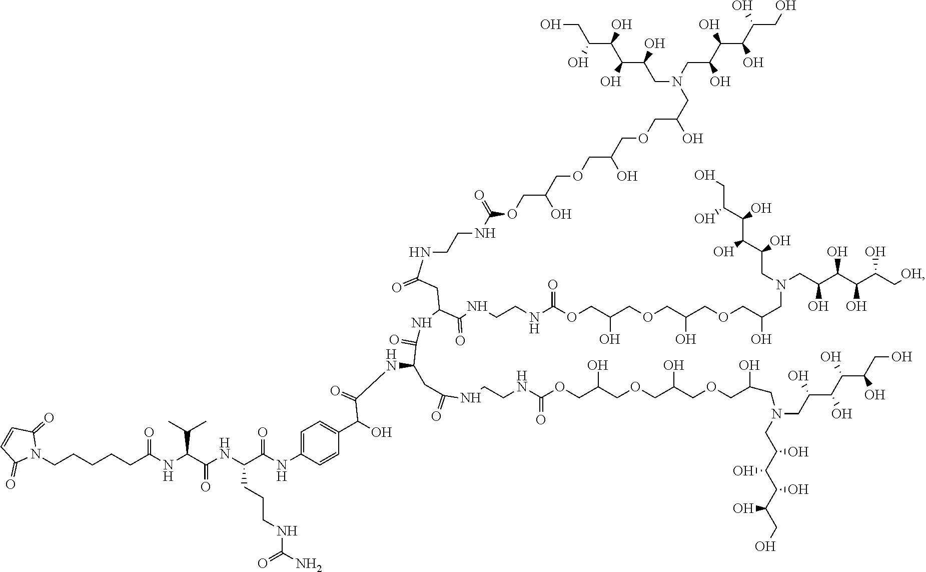

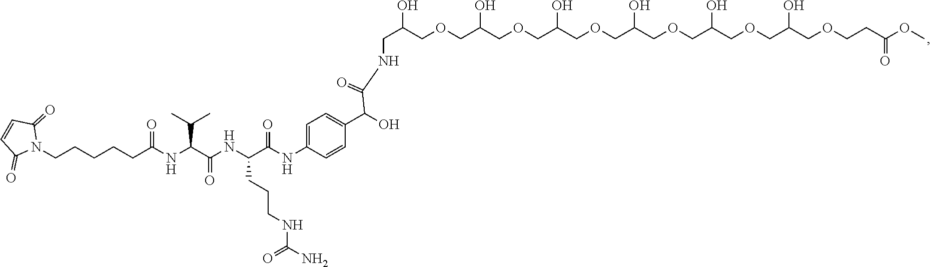 [SMILES: COC(=O)CCOCC(O)COCC(O)COCC(O)COCC(O)COCC(O)COCC(O)CNC(=O)C(O)c1ccc(NC(=O)[C@H](CCCNC(N)=O)NC(=O)[C@@H](NC(=O)CCCCCN2C(=O)C=CC2=O)C(C)C)cc1]

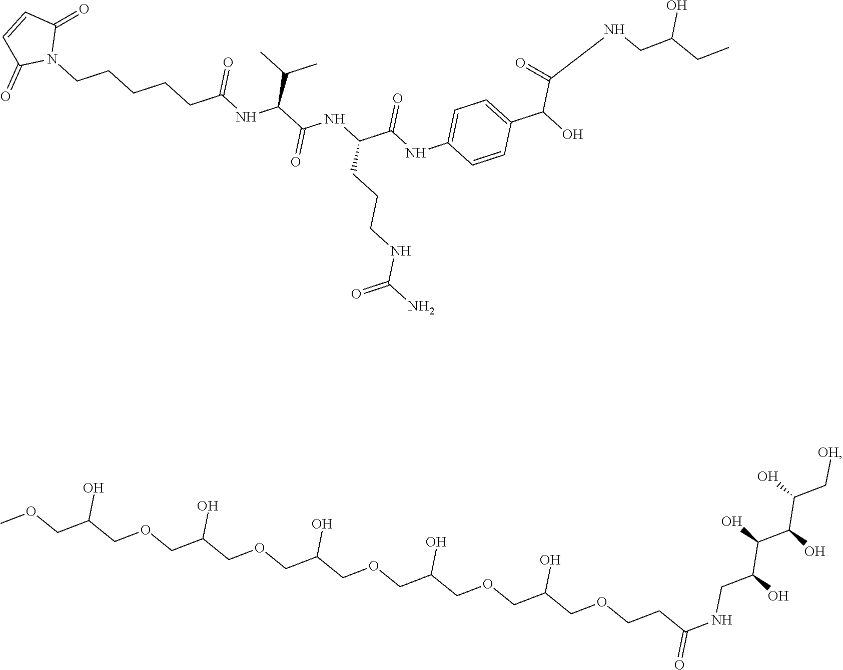

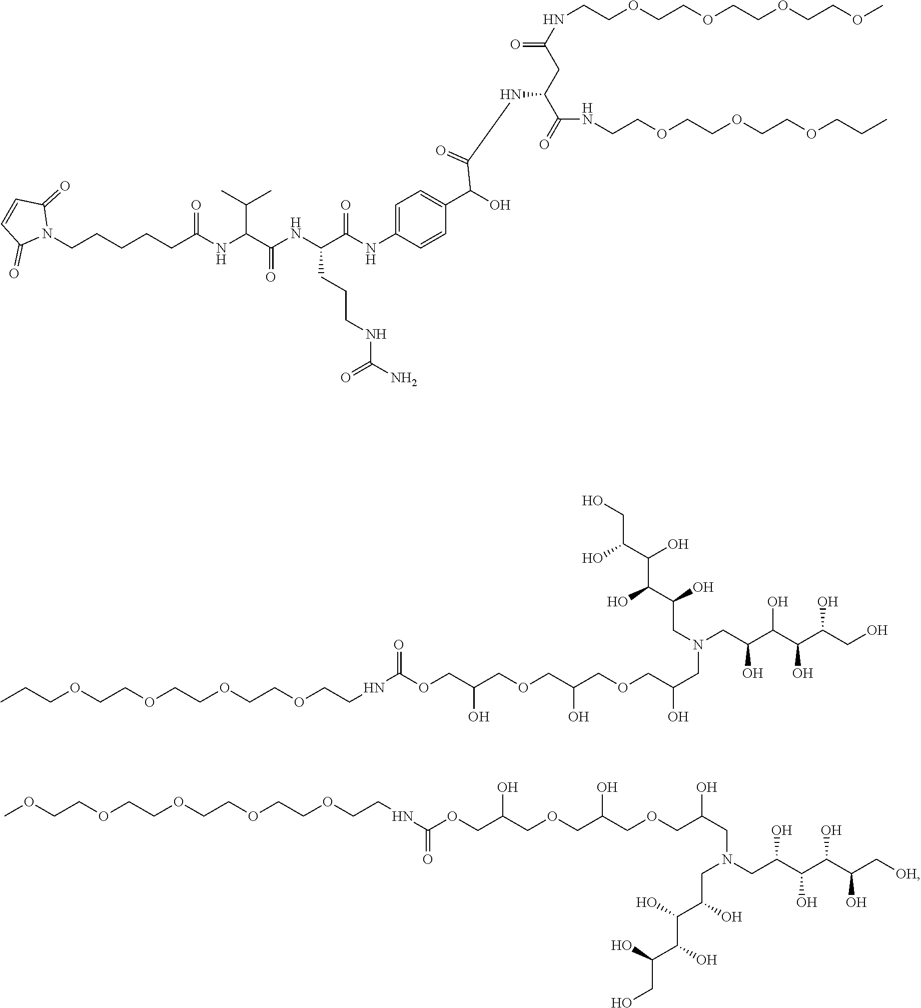

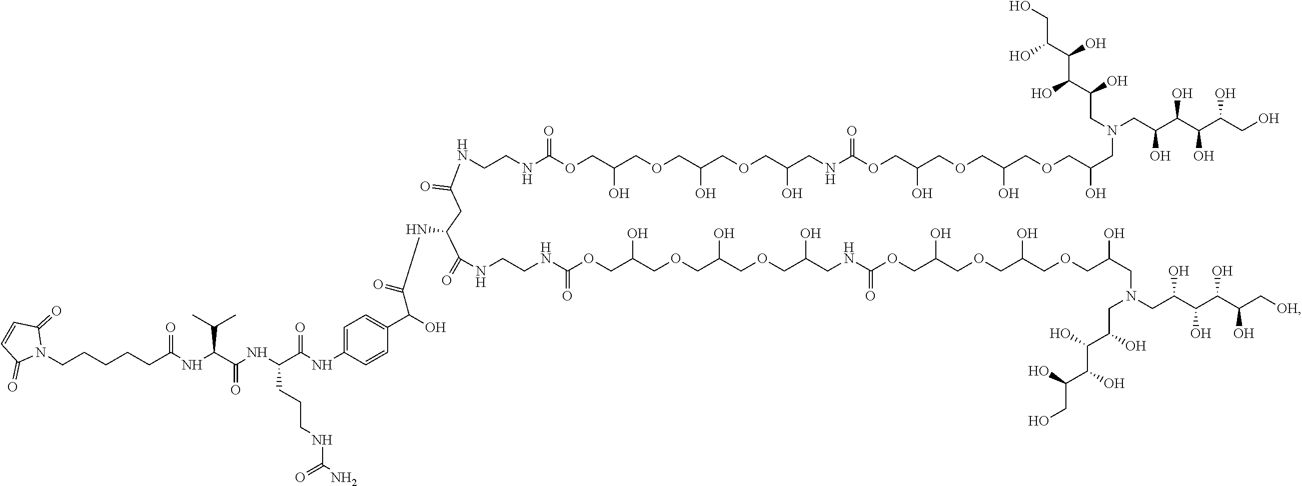 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCCNC(=O)OCC(O)COCC(O)COCC(O)CNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)CNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)cc1]

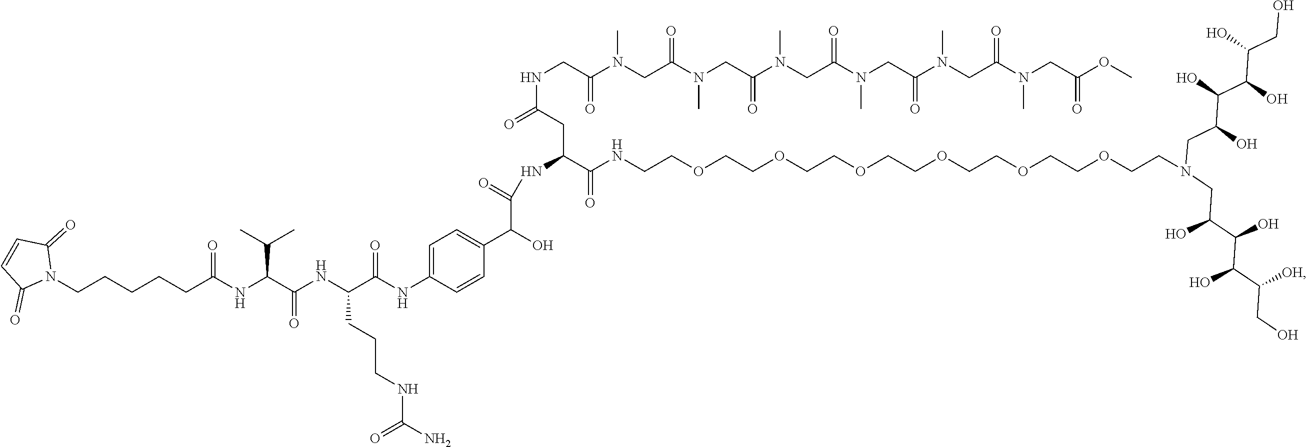 [SMILES: COC(=O)CN(C)C(=O)CN(C)C(=O)CN(C)C(=O)CN(C)C(=O)CN(C)C(=O)CN(C)C(=O)CNC(=O)C[C@H](NC(=O)C(O)c1ccc(NC(=O)[C@H](CCCNC(N)=O)NC(=O)[C@@H](NC(=O)CCCCCN2C(=O)C=CC2=O)C(C)C)cc1)C(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO]

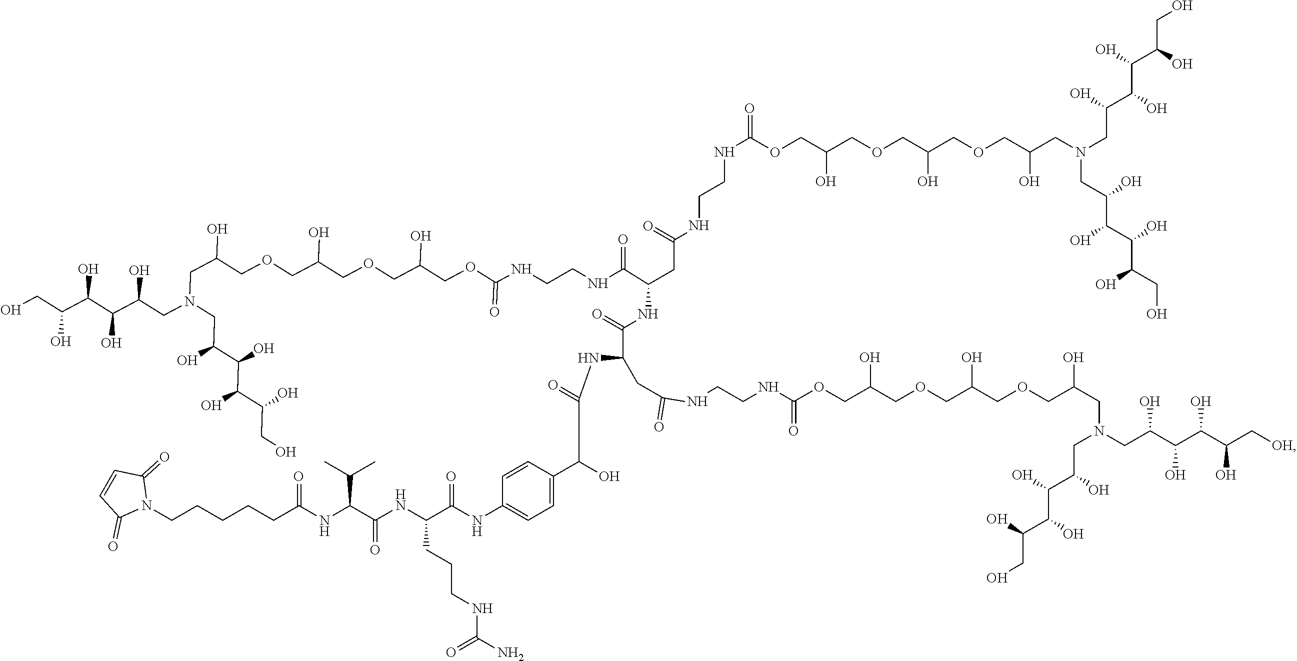 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)N[C@@H](CC(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)cc1]

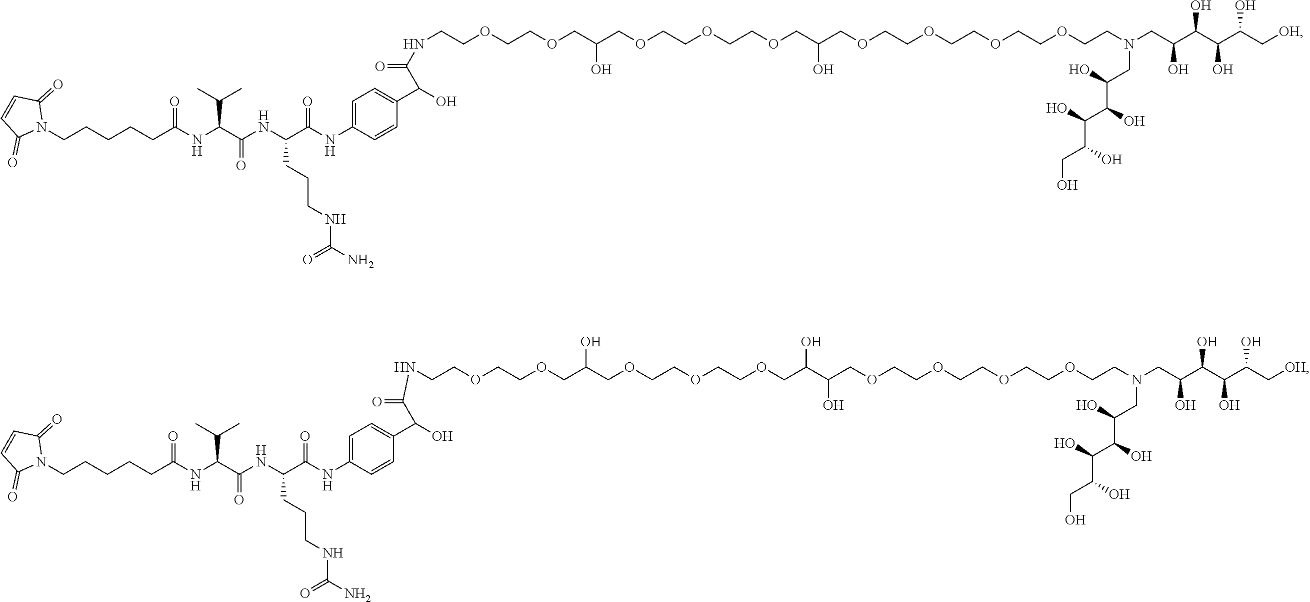 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCCOCCOCC(O)COCCOCCOCC(O)COCCOCCOCCOCCN(C[C@H](O)[C@@H](O)[C@H](O)[C@H](O)CO)C[C@H](O)[C@@H](O)[C@H](O)[C@H](O)CO)cc1 ; CC(C)[C@H](NC(=O)CCCCCN1C(=O)C=CC1=O)C(=O)N[C@@H](CCCNC(N)=O)C(=O)Nc1ccc(C(O)C(=O)NCCOCCOCC(O)COCCOCCOCC(O)C(O)COCCOCCOCCOCCN(C[C@H](O)[C@@H](O)[C@H](O)[C@H](O)CO)C[C@H](O)[C@@H](O)[C@H](O)[C@H](O)CO)cc1]

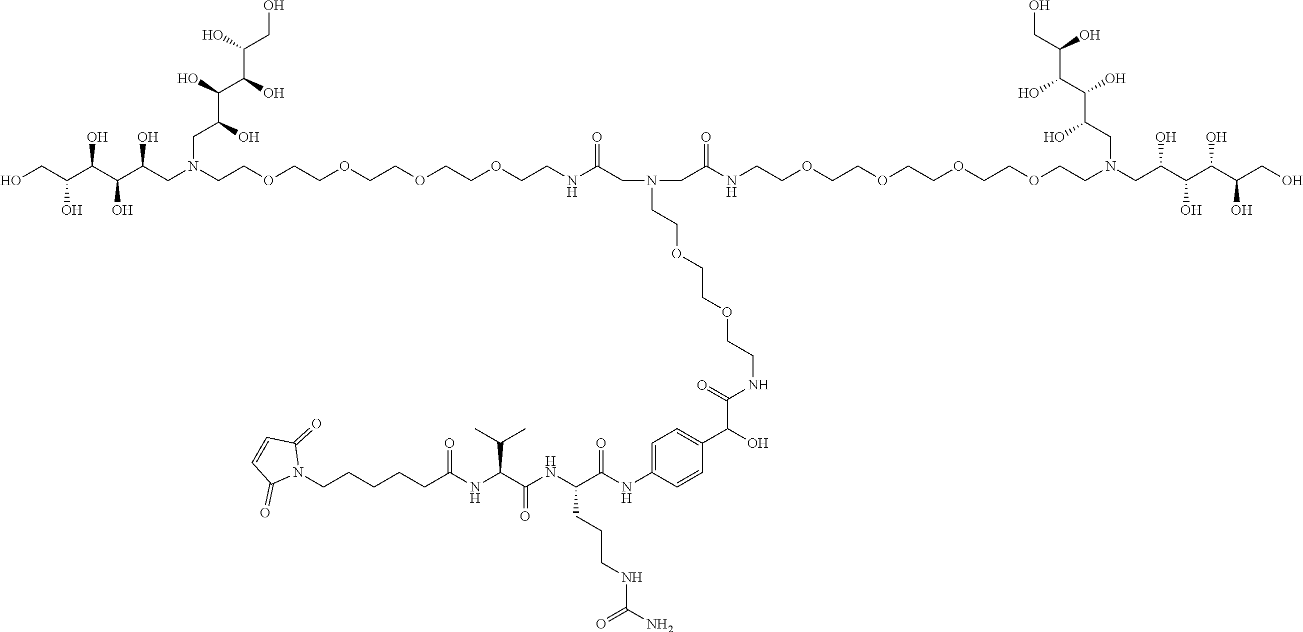 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCCOCCOCCN(CC(=O)NCCOCCOCCOCCOCCN(C[C@H](O)[C@@H](O)[C@H](O)[C@H](O)CO)C[C@H](O)[C@@H](O)[C@H](O)[C@H](O)CO)CC(=O)NCCOCCOCCOCCOCCN(C[C@H](O)[C@@H](O)[C@H](O)[C@H](O)CO)C[C@H](O)[C@@H](O)[C@H](O)[C@H](O)CO)cc1]

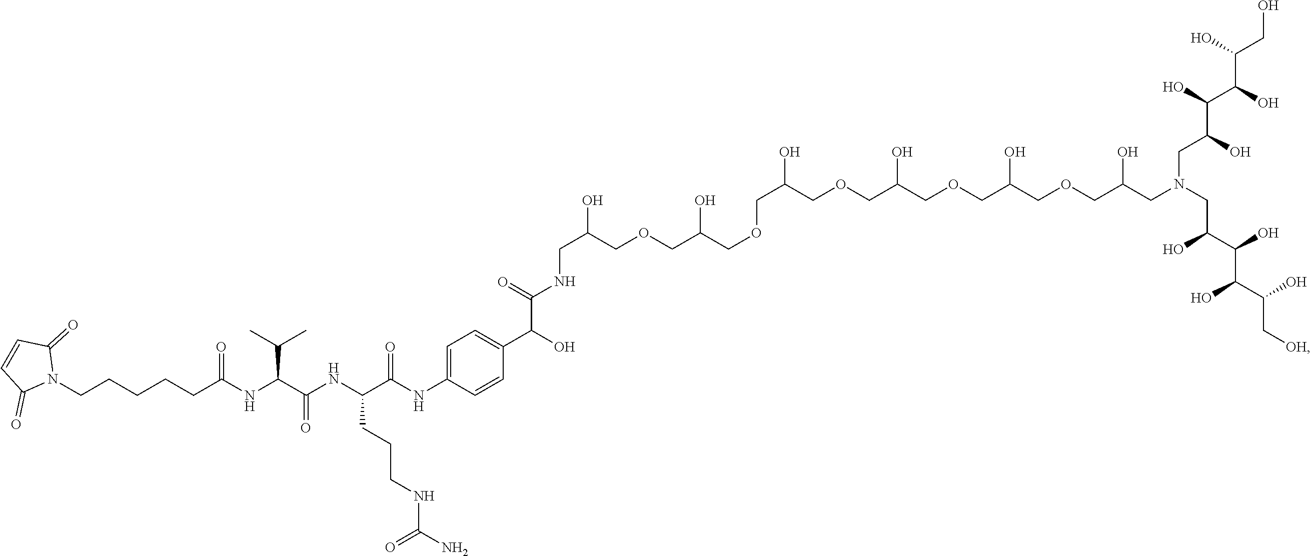 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)cc1]

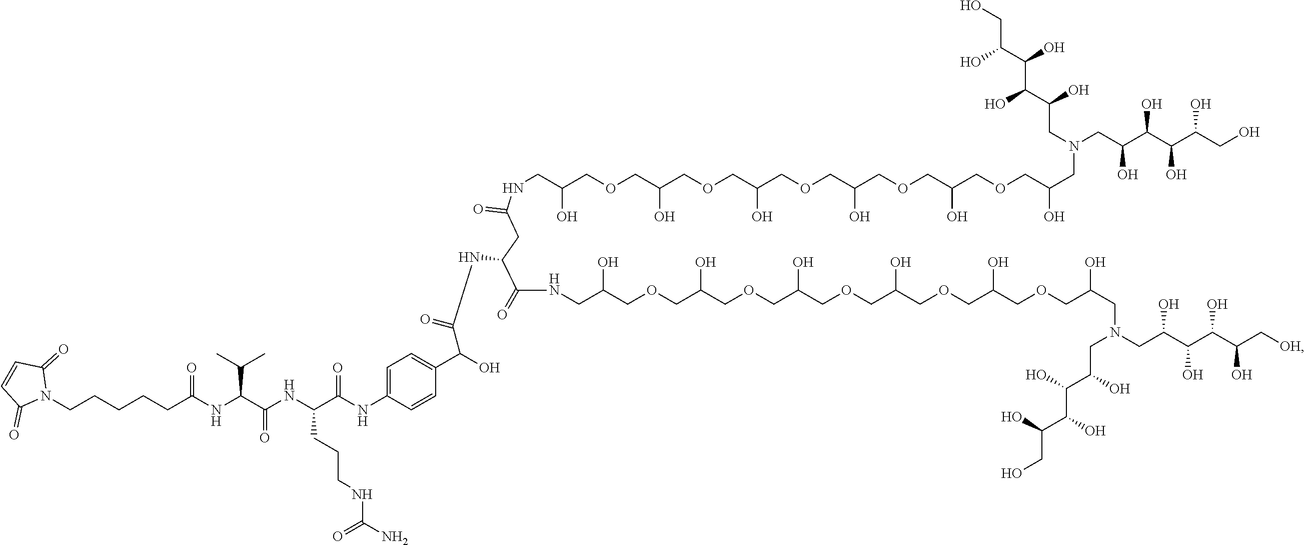 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)cc1]

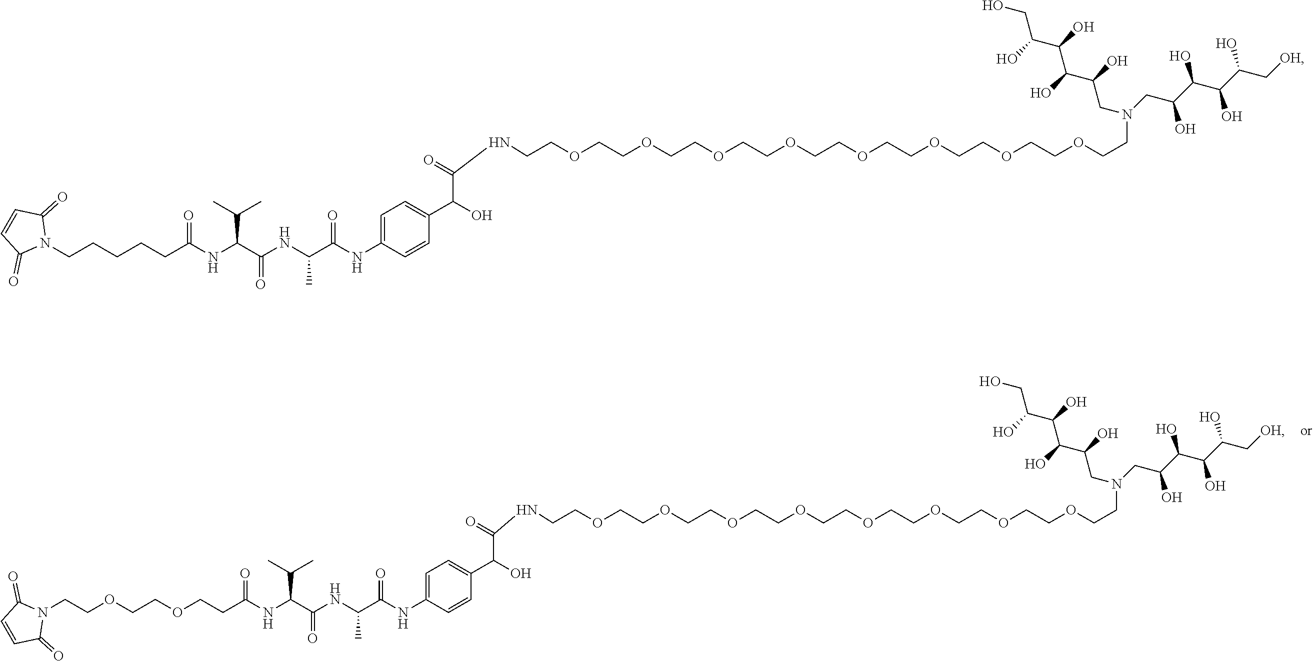 [SMILES: CC(C)[C@H](NC(=O)CCCCCN1C(=O)C=CC1=O)C(=O)N[C@@H](C)C(=O)Nc1ccc(C(O)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1 ; CC(C)[C@H](NC(=O)CCOCCOCCN1C(=O)C=CC1=O)C(=O)N[C@@H](C)C(=O)Nc1ccc(C(O)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

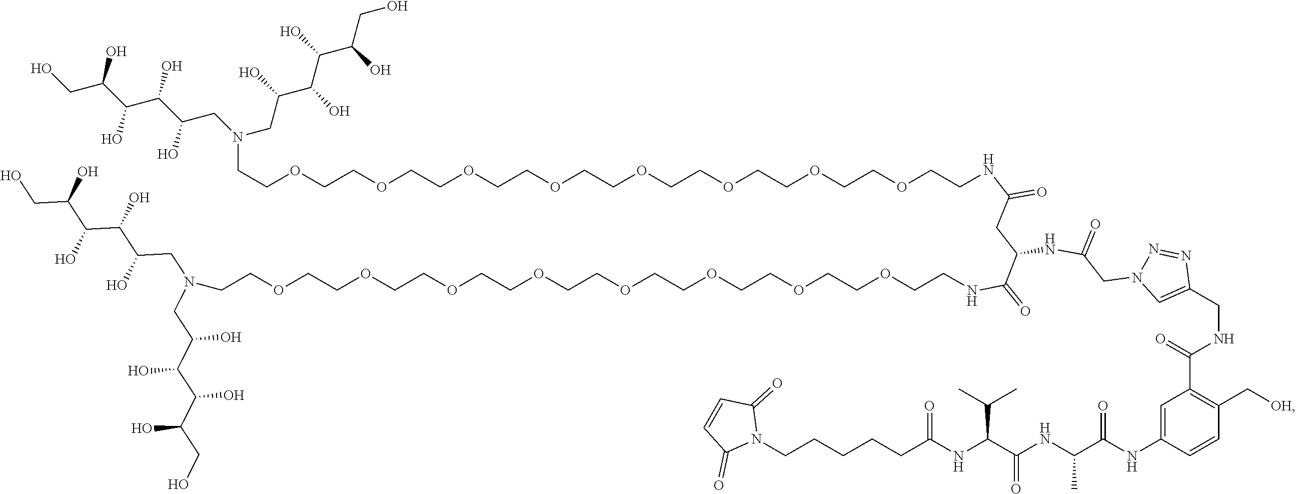 [SMILES: CC(C)[C@H](NC(=O)CCCCCN1C(=O)C=CC1=O)C(=O)N[C@@H](C)C(=O)Nc1ccc(CO)c(C(=O)NCc2cn(CC(=O)N[C@@H](CC(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)nn2)c1]

wherein the H on the benzylic OH is optionally replaced with a bond to the at least one Drug unit or to the linking group attached to the at least one Drug unit.

Embodiment 85. A Drug-Linker compound, comprising a Linker compound of any one of Embodiments 1-84 attached to the at least one Drug unit, or attached to the linking group attached to the at least one Drug unit, at the attachment site.

Embodiment 86. The Drug-Linker of Embodiment 85, wherein the Drug unit is selected from a cytotoxic agent, an immune modulatory agent, a nucleic acid, a growth inhibitory agent, a PROTAC, a toxin, a radioactive isotope and a chelating ligand.

Embodiment 87. The Drug-Linker of Embodiment 86, wherein the Drug unit is a cytotoxic agent.

Embodiment 88. The Drug-Linker of Embodiment 87, wherein the cytotoxic agent is selected from the group consisting of an auristatin, a maytansinoid, a camptothecin, a duocarmycin, and a calicheamicin.

Embodiment 89. The Drug-Linker of Embodiment 88, wherein the cytotoxic agent is an auristatin.

Embodiment 90. The Drug-Linker of Embodiment 89, wherein the cytotoxic agent is MMAE or MMAF.

Embodiment 91. The Drug-Linker of Embodiment 88, wherein the cytotoxic agent is a camptothecin.

Embodiment 92. The Drug-Linker of Embodiment 91, wherein the cytotoxic agent is exatecan or SN-38 or DxD.

Embodiment 93. The Drug-Linker of Embodiment 92, wherein the cytotoxic agent is exatecan.

Embodiment 94. The Drug-Linker of Embodiment 88, wherein the cytotoxic agent is a calicheamicin.

Embodiment 95. The Drug-Linker of Embodiment 88, wherein the cytotoxic agent is a maytansinoid.

Embodiment 96. The Drug-Linker of Embodiment 95, wherein the maytansinoid is maytansine, maytansinol or ansamatocin-2.

Embodiment 97. The Drug-Linker of Embodiment 86, wherein the Drug unit is an immune modulatory agent.

Embodiment 98. The Drug-Linker of Embodiment 97, wherein the immune modulatory agent is selected from a TRL7 agonist, a TLR8 agonist, a STING agonist, or a RIG-1 agonist.

Embodiment 99. The Drug-Linker of Embodiment 98, wherein the immune modulatory agent is an TLR7 agonist.

Embodiment 100. The Drug-Linker of Embodiment 99, wherein the TLR7 agonist is an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, a benzonaphthyridine, a guanosine analog, an adenosine analog, a thymidine homopolymer, ssRNA, CpG-A, PolyG10, or PolyG3.

Embodiment 101. The Drug-Linker of Embodiment 98, wherein the immune modulatory agent is a TLR8 agonist.

Embodiment 102. The Drug-Linker of Embodiment 101, wherein the TLR8 agonist is selected from an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine or a ssRNA.

Embodiment 103. The Drug-Linker of Embodiment 97, wherein the immune modulatory agent is a STING agonist.

Embodiment 104. The Drug-Linker of Embodiment 98, wherein the immune modulatory agent is a RIG-1 agonist.

Embodiment 105. The Drug-Linker of Embodiment 104, wherein the RIG-1 agonist is selected from KIN1148, SB-9200, KIN700, KIN600, KIN500, KIN100, KIN101, KIN400 and KIN2000.

Embodiment 106. The Drug-Linker of Embodiment 86, wherein the Drug unit is a chelating ligand.

Embodiment 107. The Drug-Linker of Embodiment 106, wherein the chelating ligand is selected from platinum (Pt), ruthenium (Ru), rhodium (Rh), gold (Au), silver (Ag), copper (Cu), molybdenum (Mo), titanium (Ti), or iridium (Ir); a radioisotope such as yittrium-88, yittrium-90, technetium-99, copper-67, rhenium-188, rhenium-186, galium-66, galium-67, indium-111, indium-114, indium-115, lutetium-177, strontium-89, sararium-153, and lead-212.

Embodiment 108. The Drug-Linker of Embodiment 85, having one of the following structure:

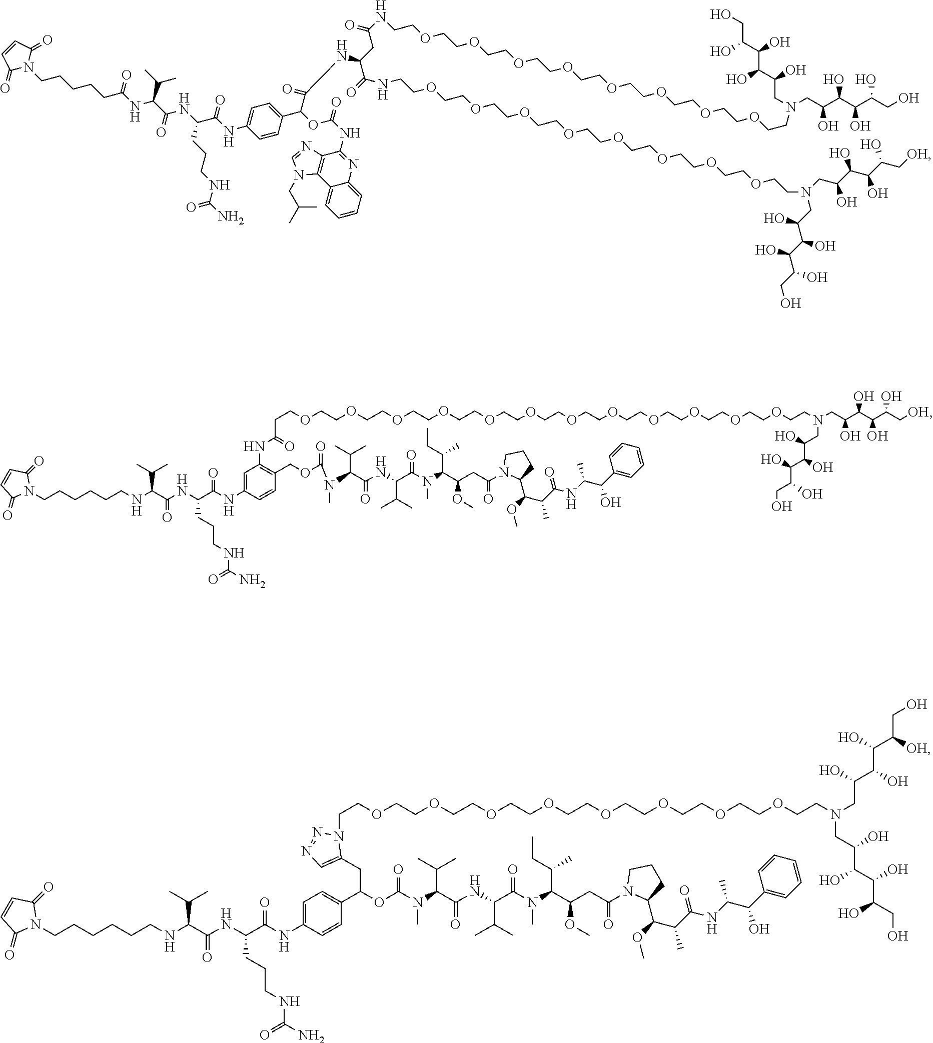

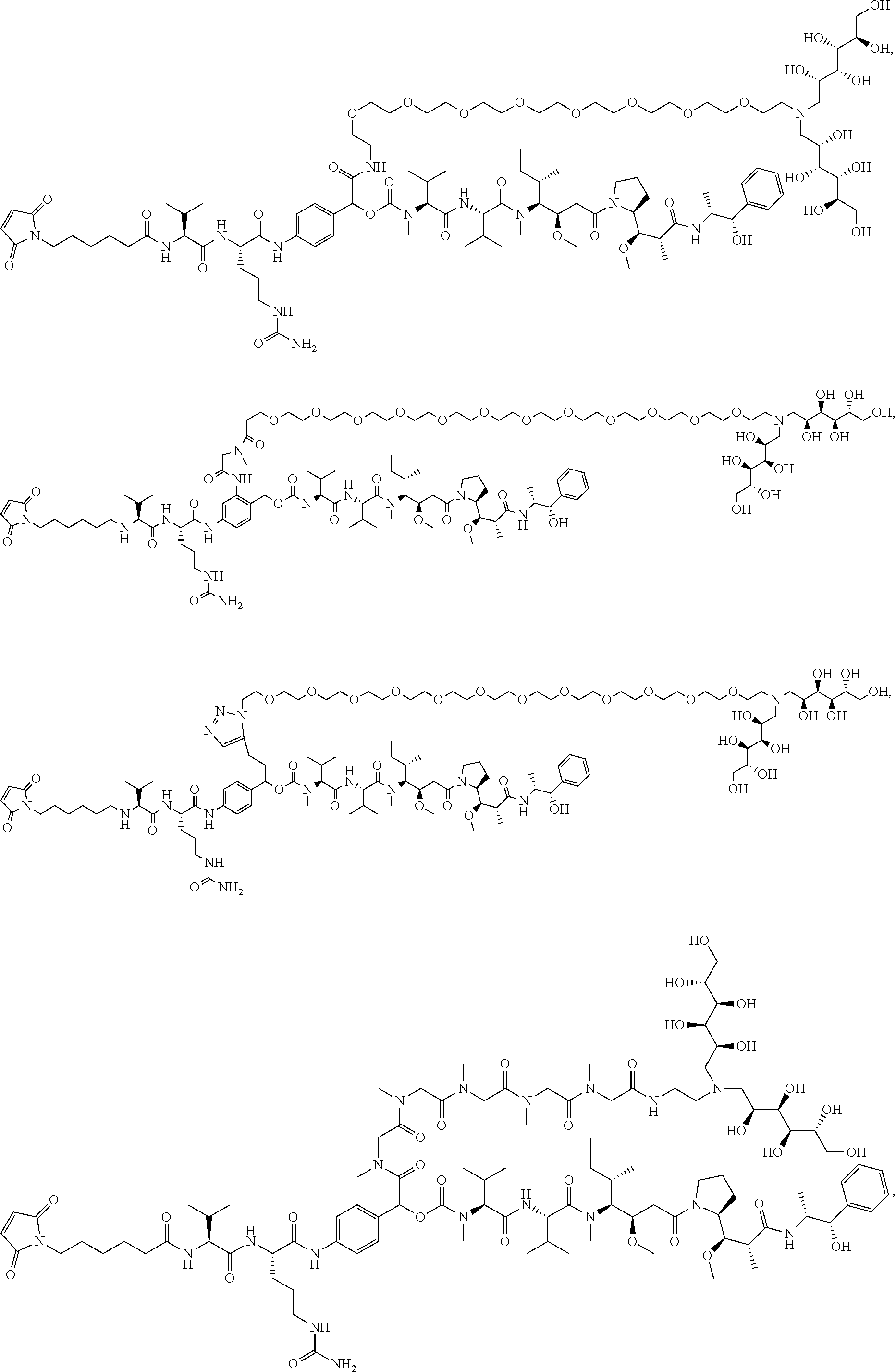

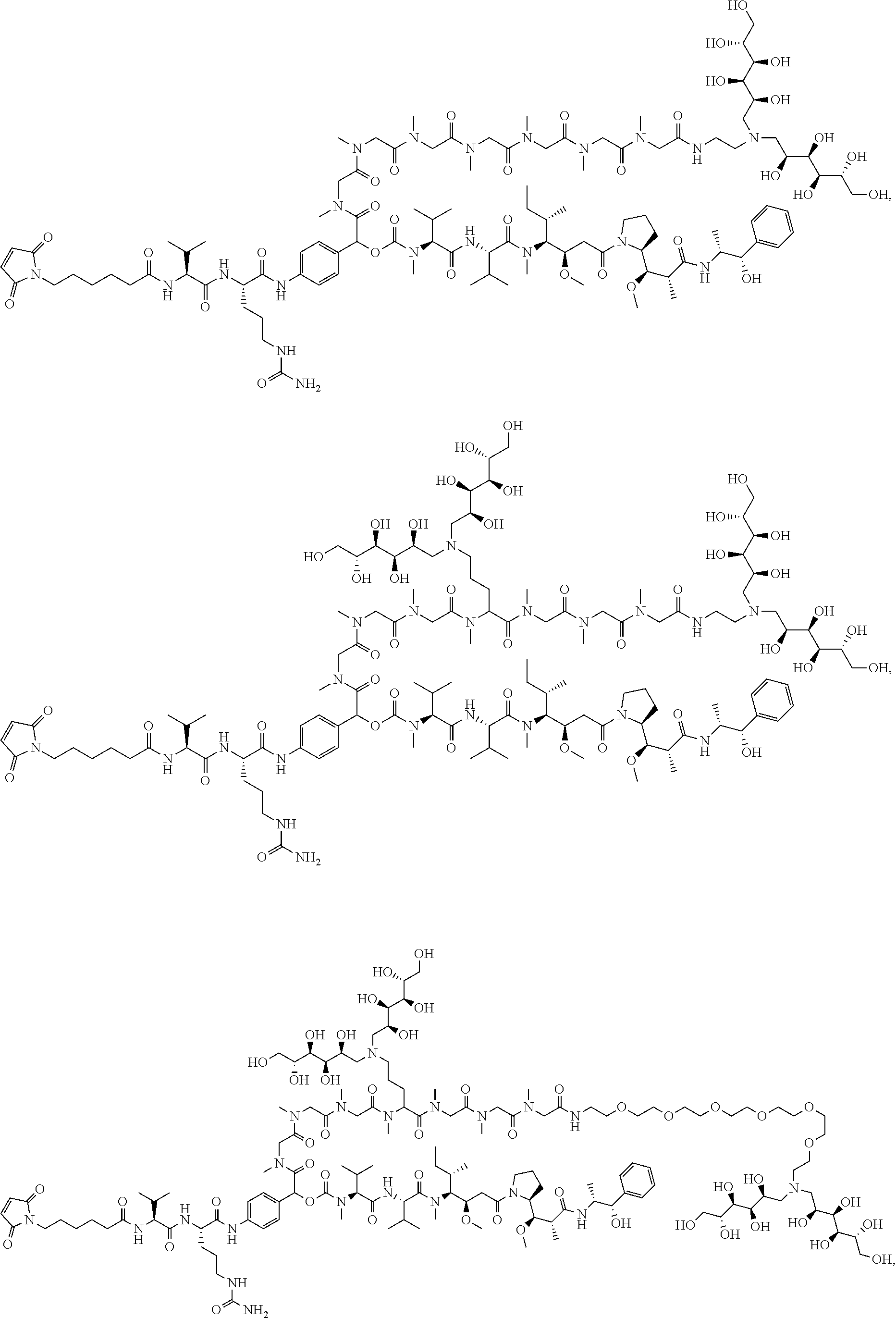

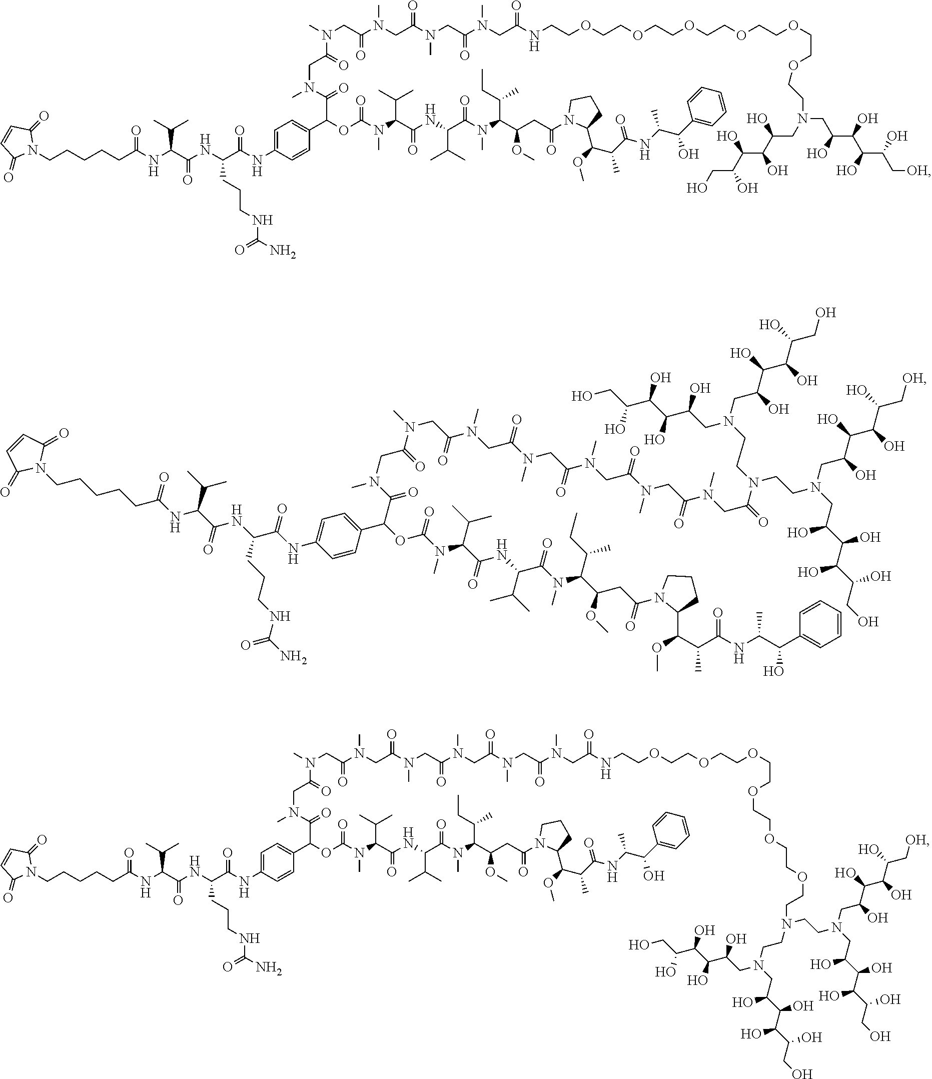

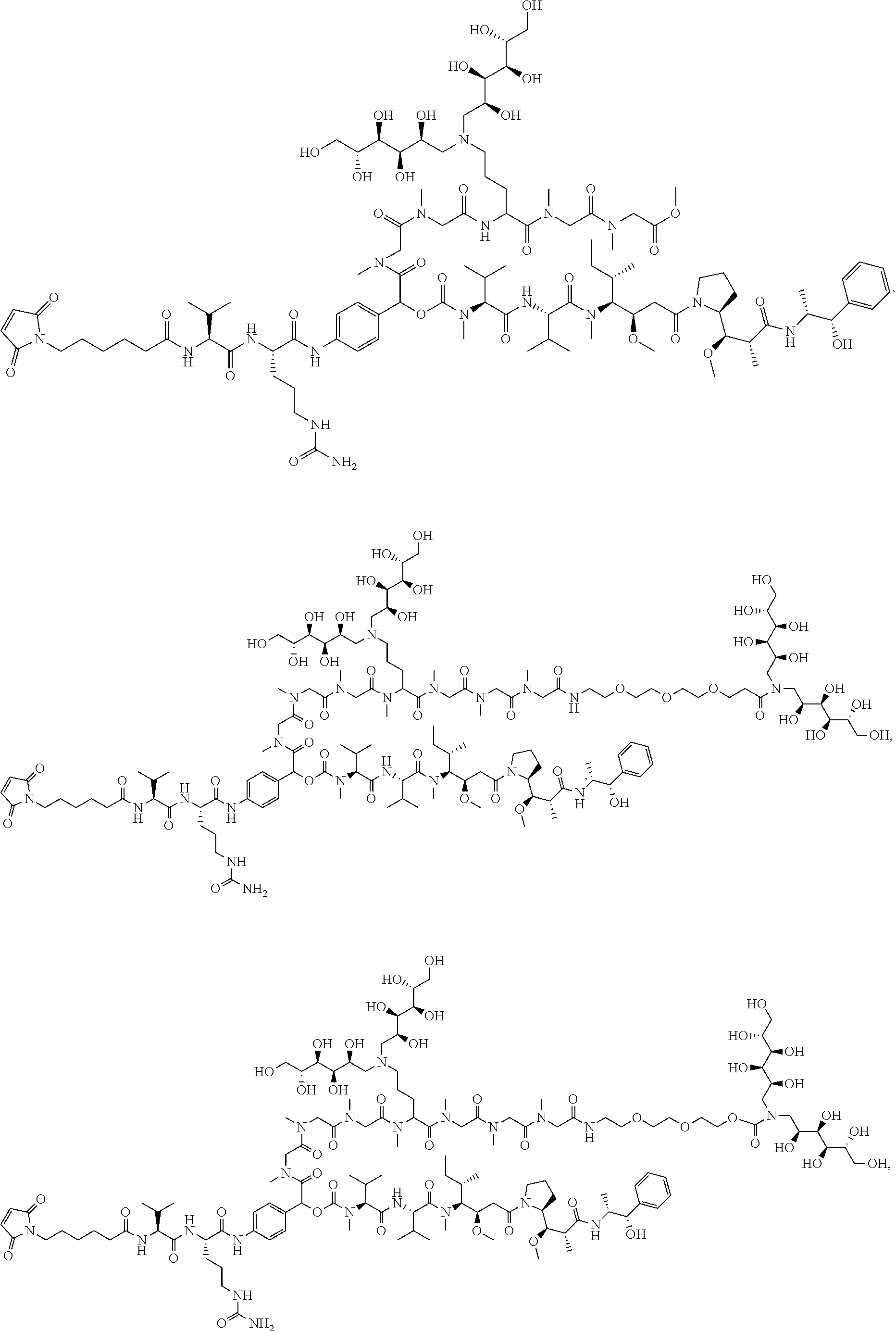

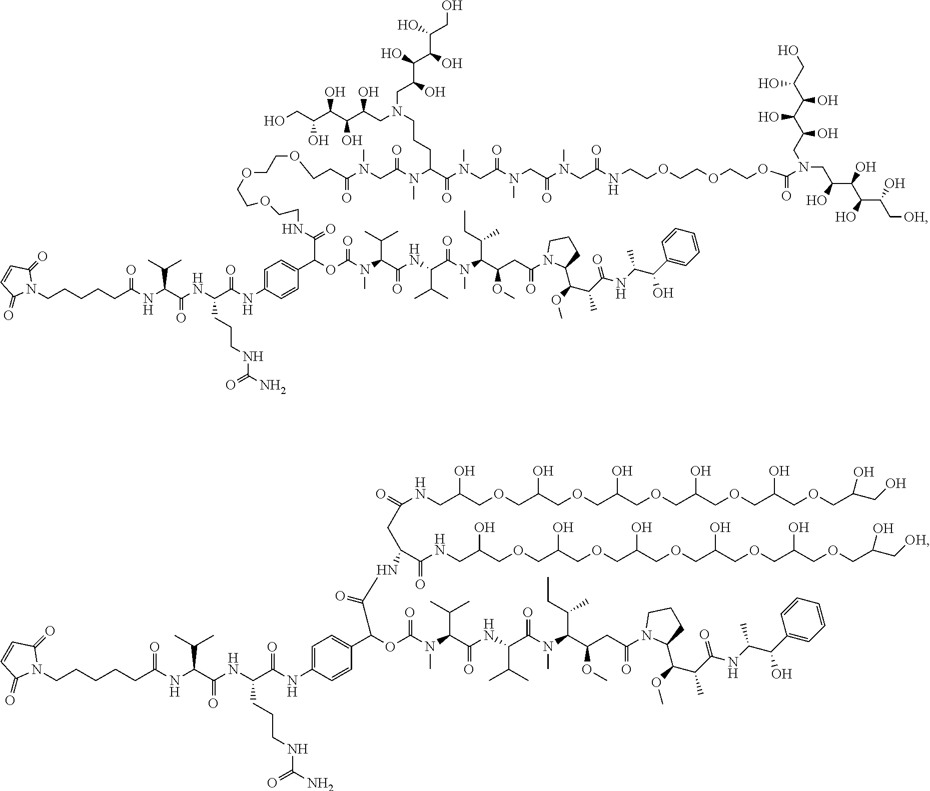

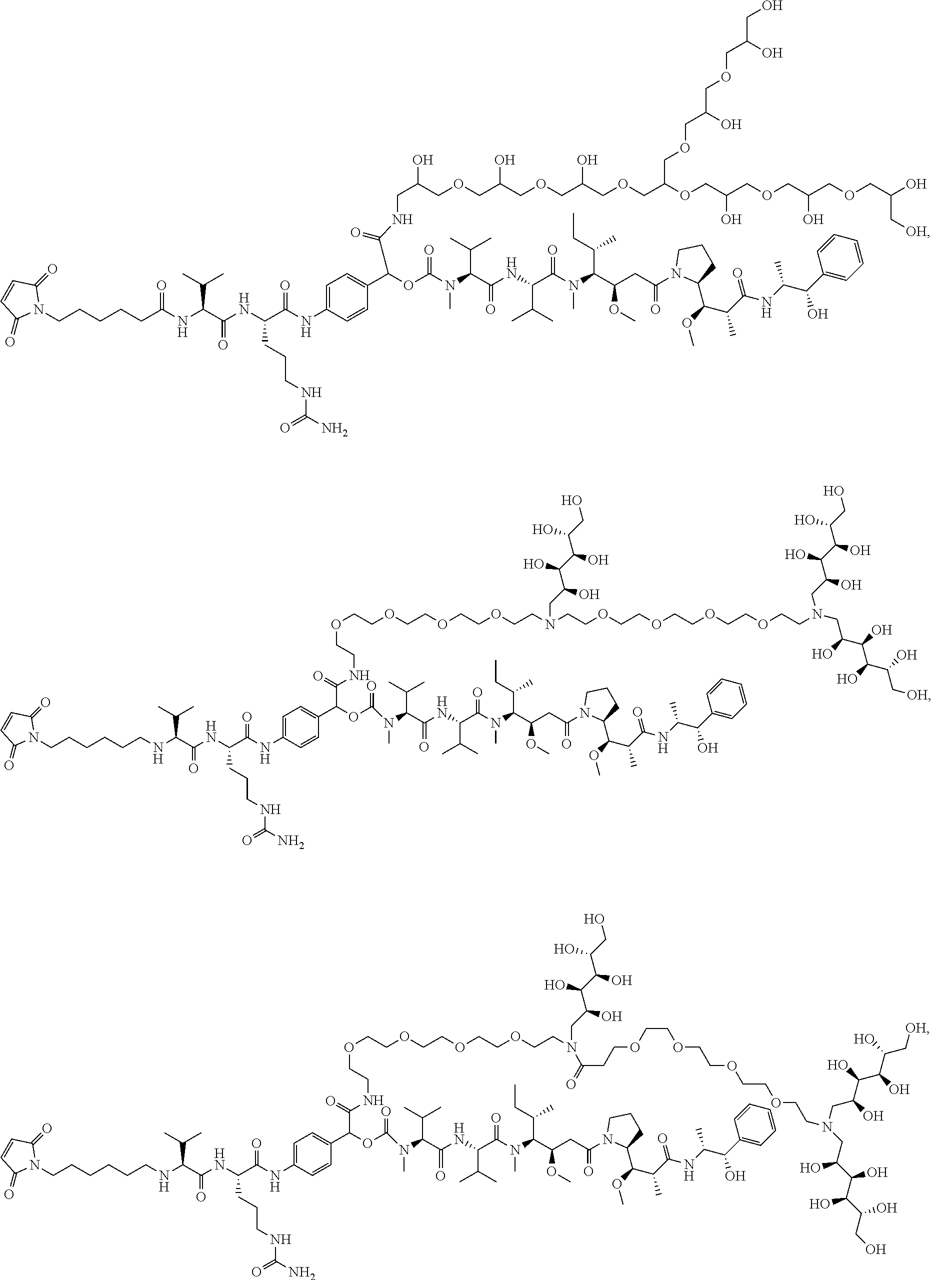

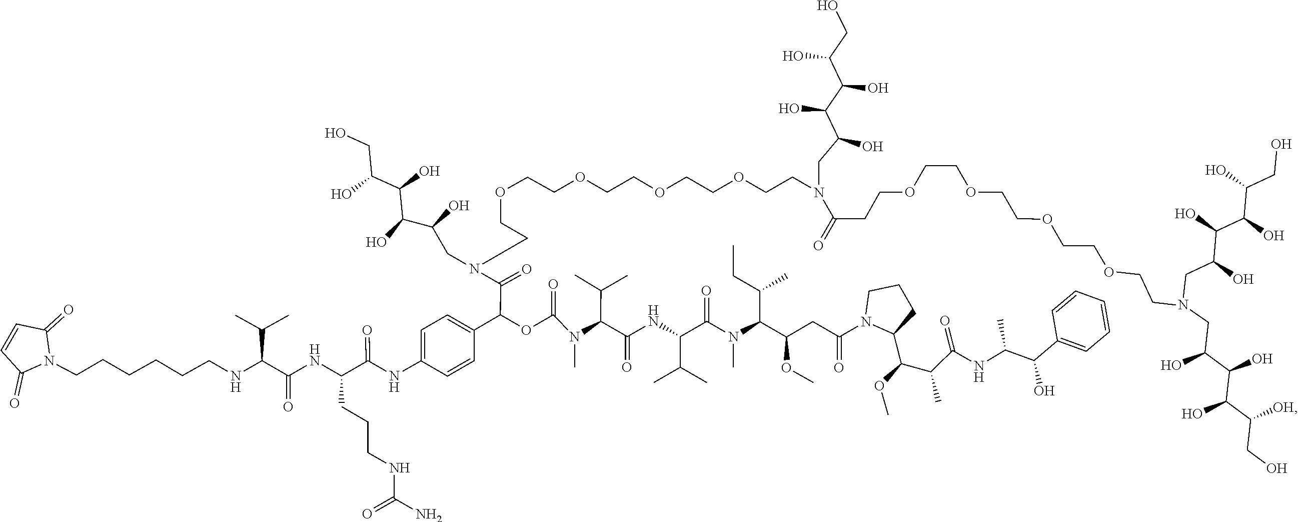 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(CCOCCOCCOCCOCCN(C[C@H](O)[C@@H](O)[C@H](O)[C@H](O)CO)C(=O)CCOCCOCCOCCOCCN(C[C@H](O)[C@@H](O)[C@H](O)[C@H](O)CO)C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NCCCCCCN2C(=O)C=CC2=O)C(C)C)cc1)C(C)C]

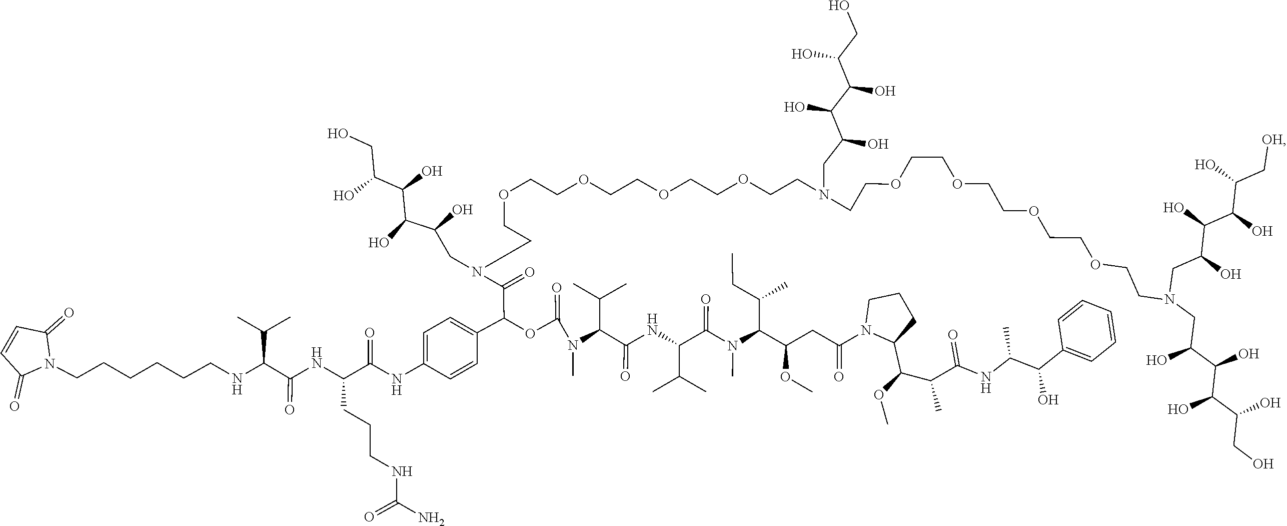 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(CCOCCOCCOCCOCCN(CCOCCOCCOCCOCCN(C[C@H](O)[C@@H](O)[C@H](O)[C@H](O)CO)C[C@H](O)[C@@H](O)[C@H](O)[C@H](O)CO)C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NCCCCCCN2C(=O)C=CC2=O)C(C)C)cc1)C(C)C]

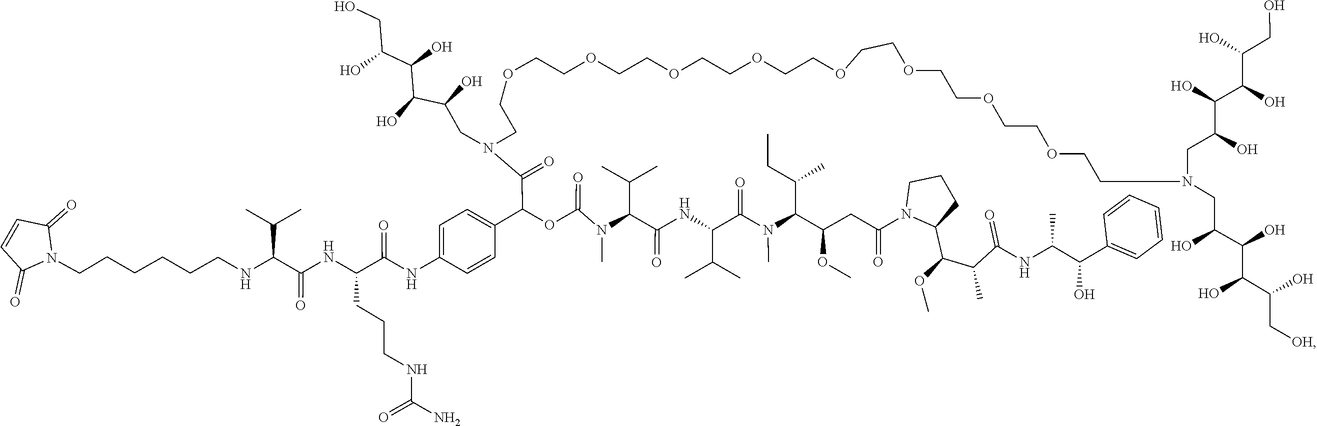 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(CCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NCCCCCCN2C(=O)C=CC2=O)C(C)C)cc1)C(C)C]

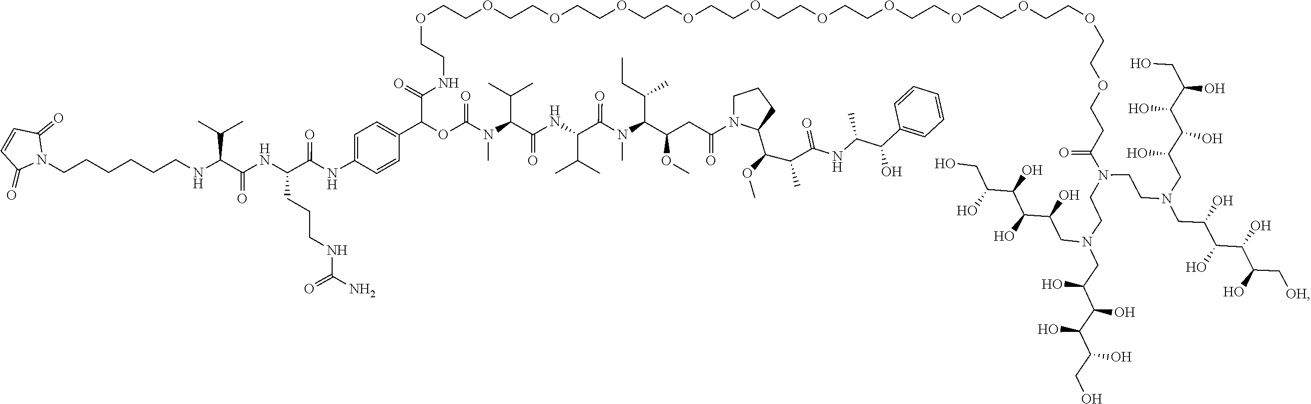 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCCOCCOCCOCCOCCOCCOCCOCCOCCOCCOCCOCCOCCC(=O)N(CCN(C[C@H](O)[C@@H](O)[C@H](O)[C@H](O)CO)C[C@H](O)[C@@H](O)[C@H](O)[C@H](O)CO)CCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NCCCCCCN2C(=O)C=CC2=O)C(C)C)cc1)C(C)C]

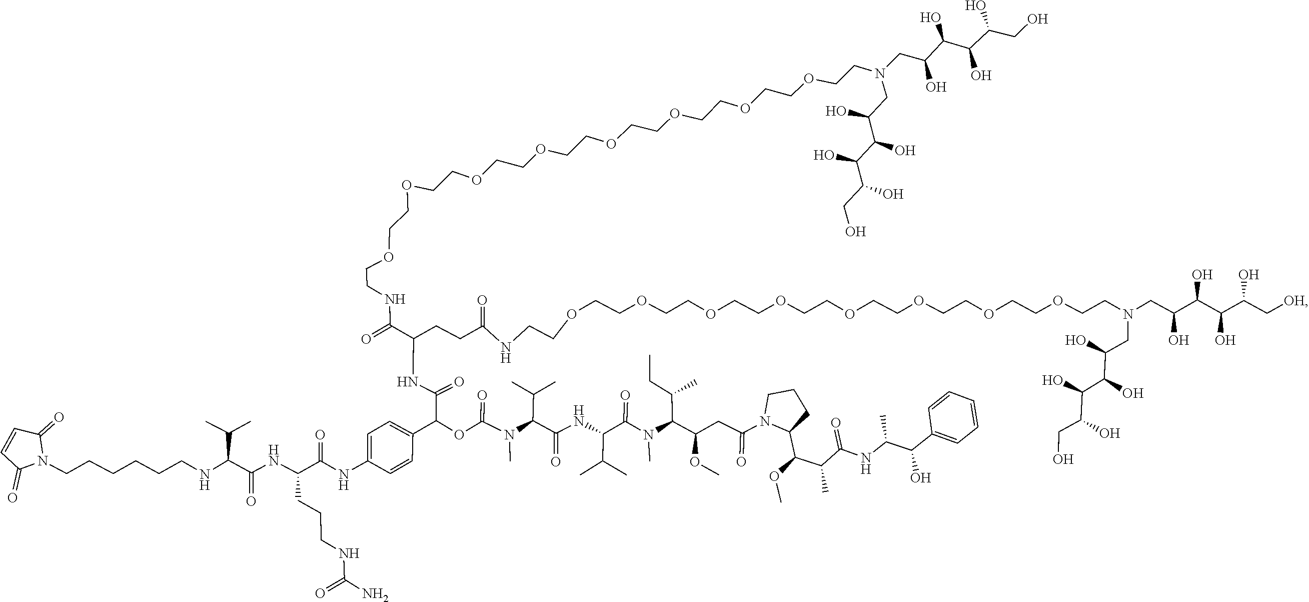 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NC(CCC(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NCCCCCCN2C(=O)C=CC2=O)C(C)C)cc1)C(C)C]

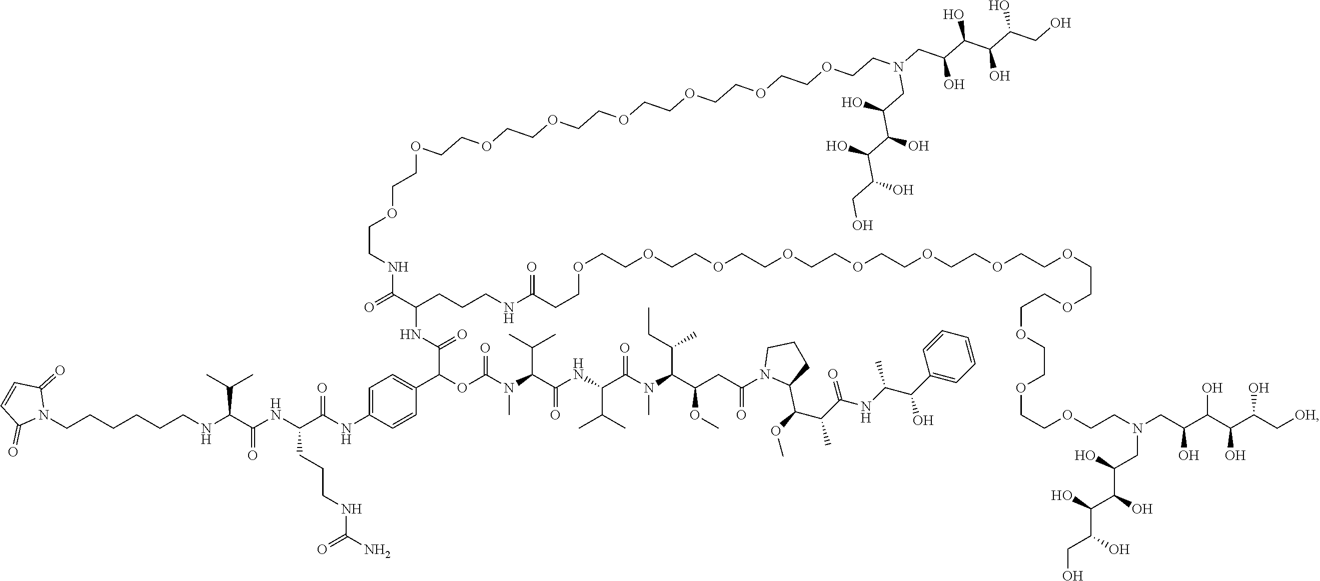 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NC(CCCNC(=O)CCOCCOCCOCCOCCOCCOCCOCCOCCOCCOCCOCCOCCN(C[C@H](O)C(O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NCCCCCCN2C(=O)C=CC2=O)C(C)C)cc1)C(C)C]

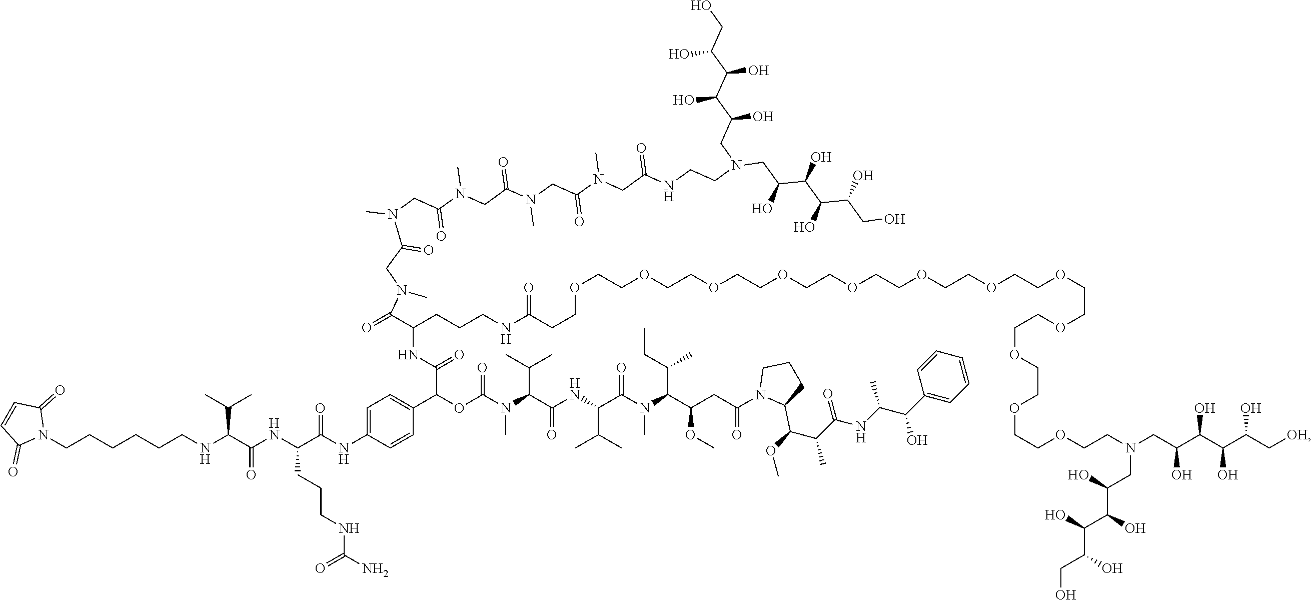 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NC(CCCNC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NCCCCCCN2C(=O)C=CC2=O)C(C)C)cc1)C(C)C]

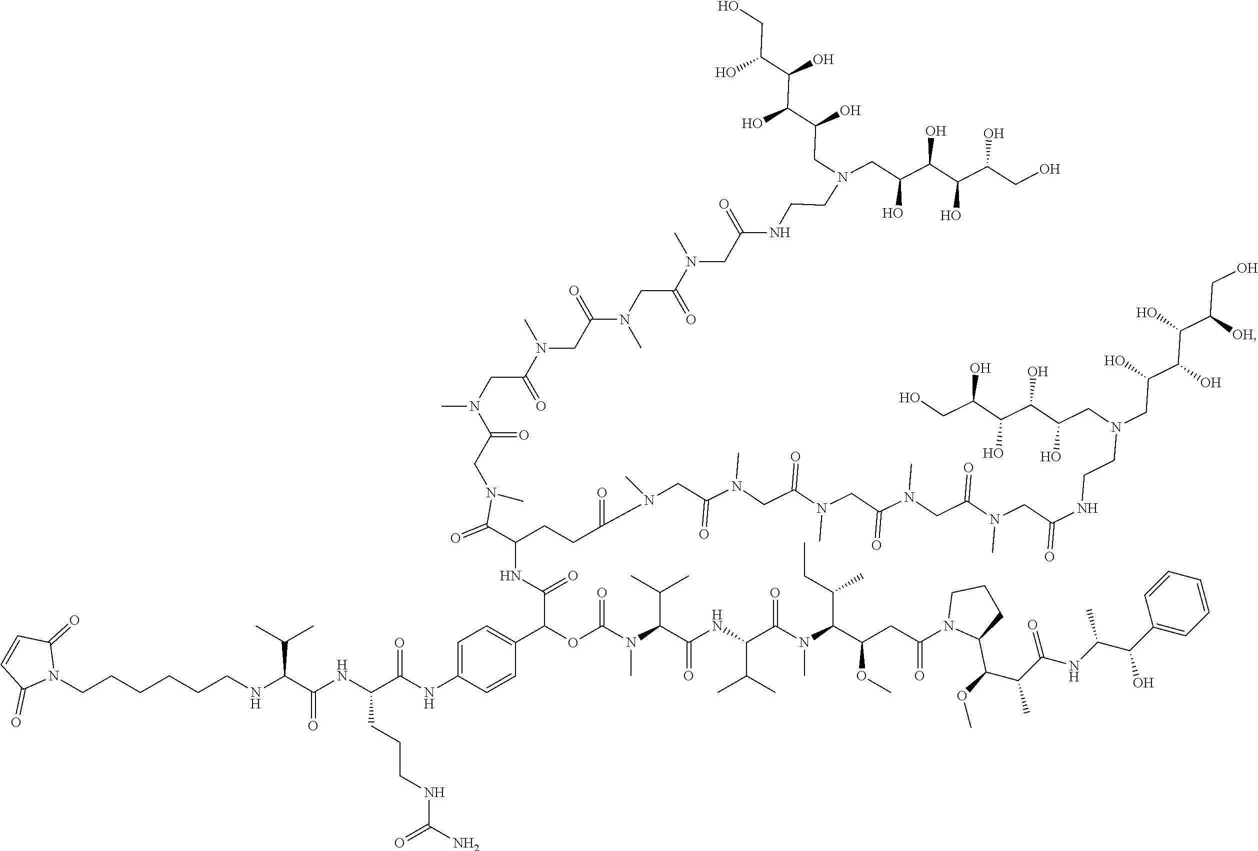 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NC(CCC(=O)N(C)CC(=O)N(C)CC(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NCCCCCCN2C(=O)C=CC2=O)C(C)C)cc1)C(C)C]

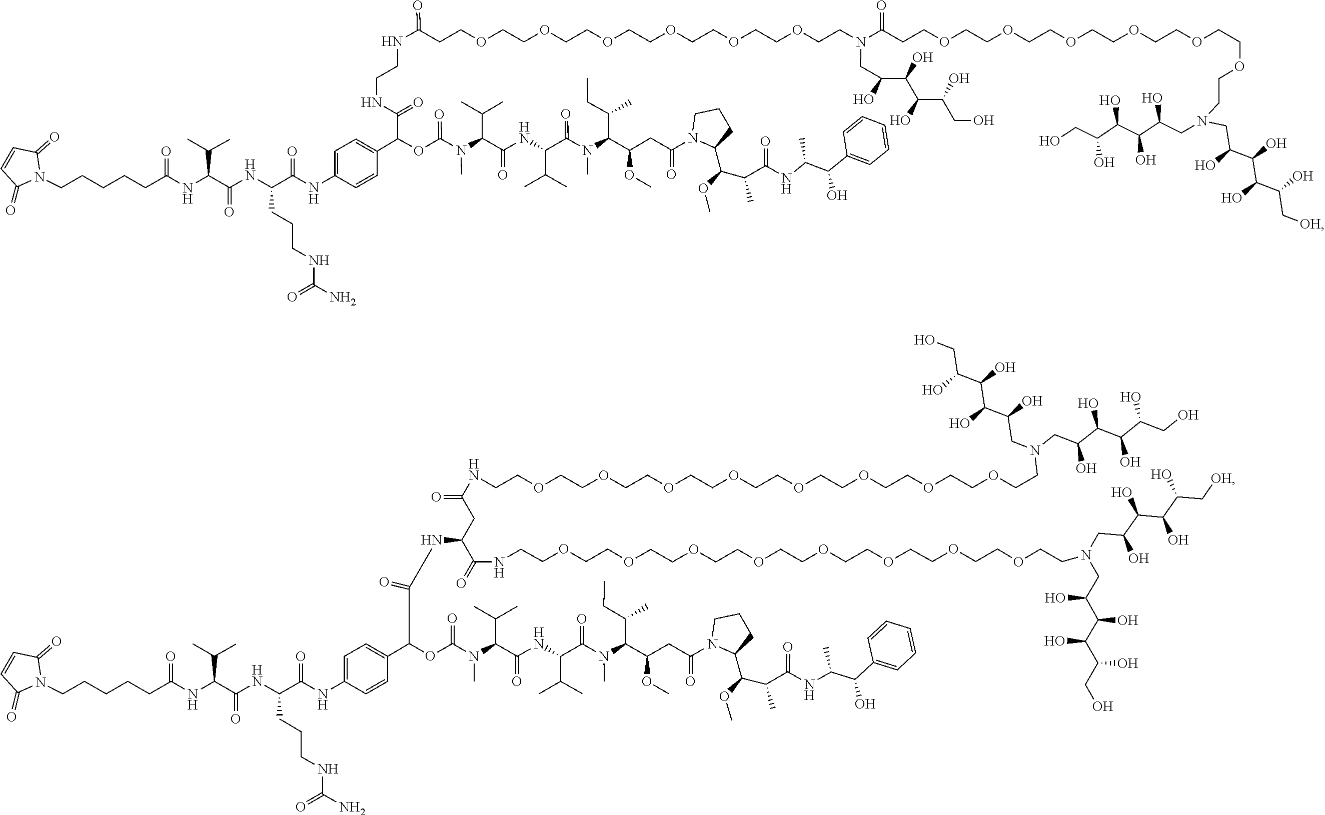

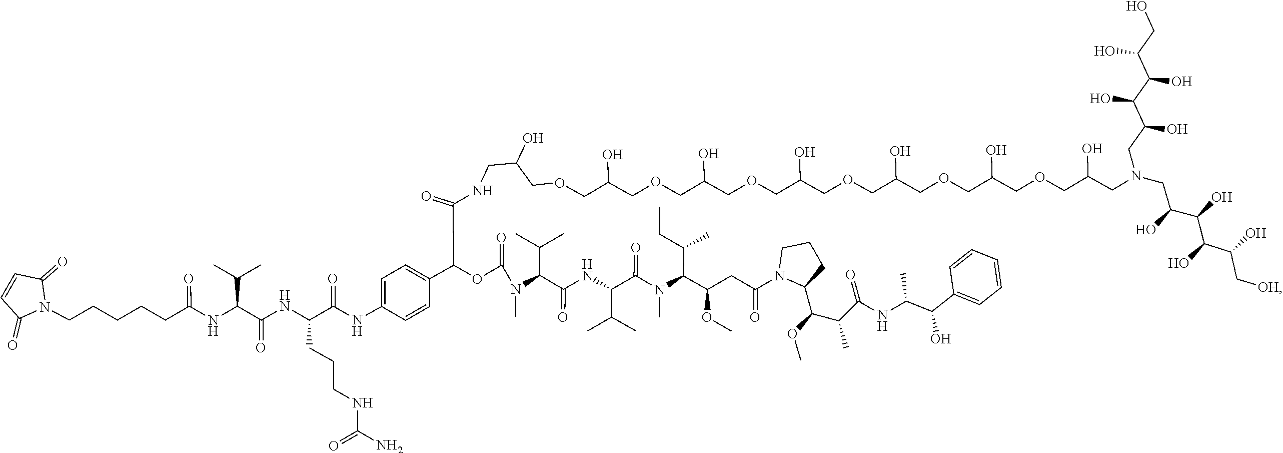 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCC(O)COCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

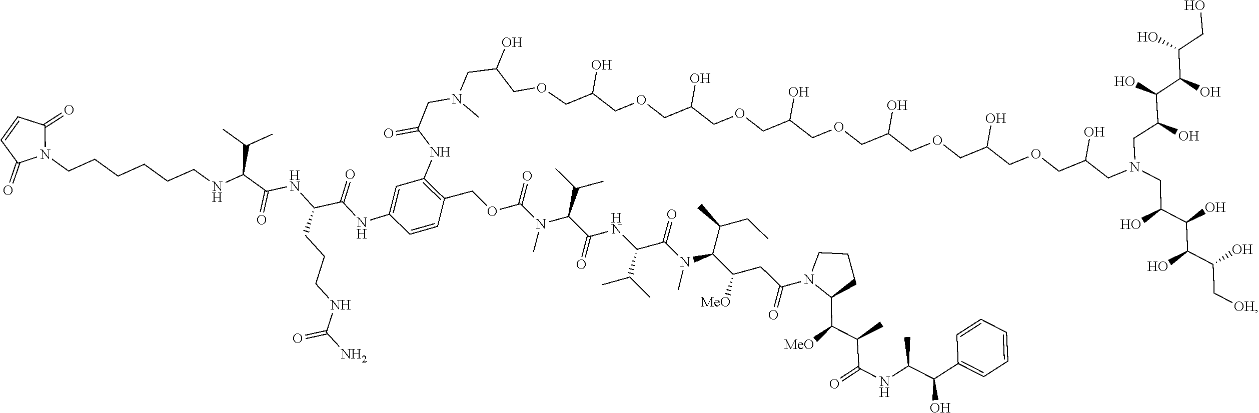 [SMILES: CC[C@H](C)[C@@H]([C@H](CC(=O)N1CCC[C@H]1[C@H](OC)[C@@H](C)C(=O)N[C@@H](C)[C@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NCCCCCCN2C(=O)C=CC2=O)C(C)C)cc1NC(=O)CN(C)CC(O)COCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)C(C)C]

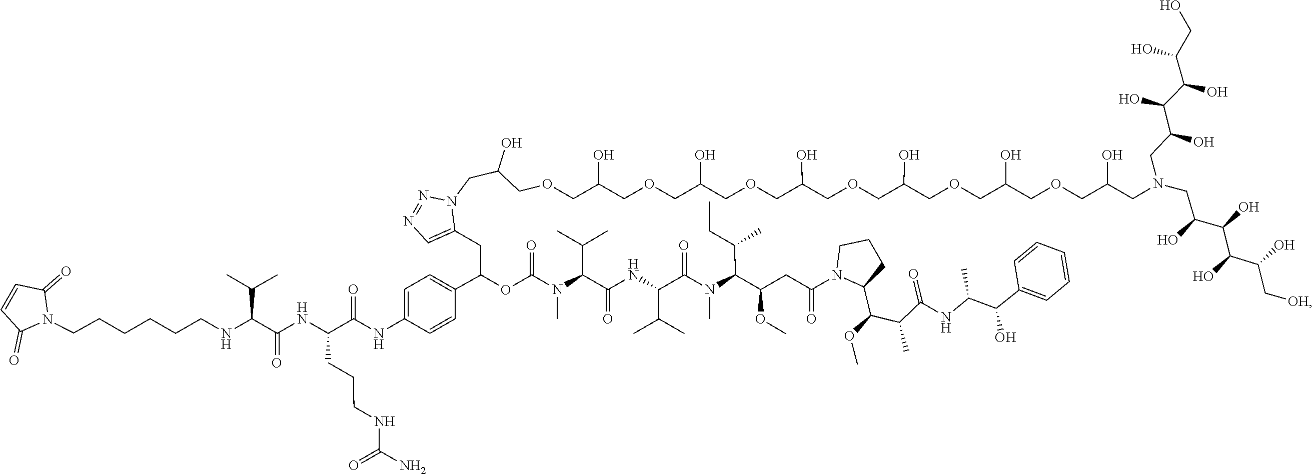 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(Cc1cnnn1CC(O)COCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NCCCCCCN2C(=O)C=CC2=O)C(C)C)cc1)C(C)C]

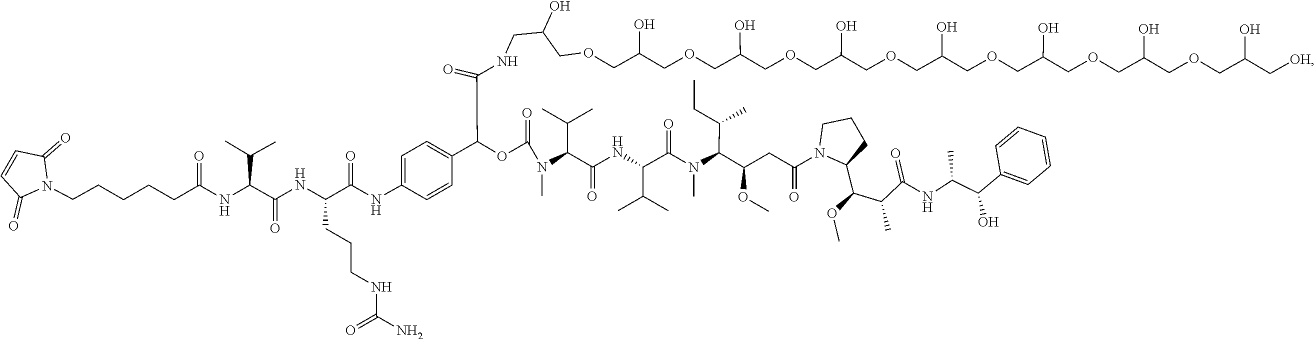 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCC(O)COCC(O)COCC(O)COCC(O)COCC(O)COCC(O)COCC(O)COCC(O)CO)c1ccc(NC(=O)[C@H](CCCNC(N)=O)NC(=O)[C@@H](NC(=O)CCCCCN2C(=O)C=CC2=O)C(C)C)cc1)C(C)C]

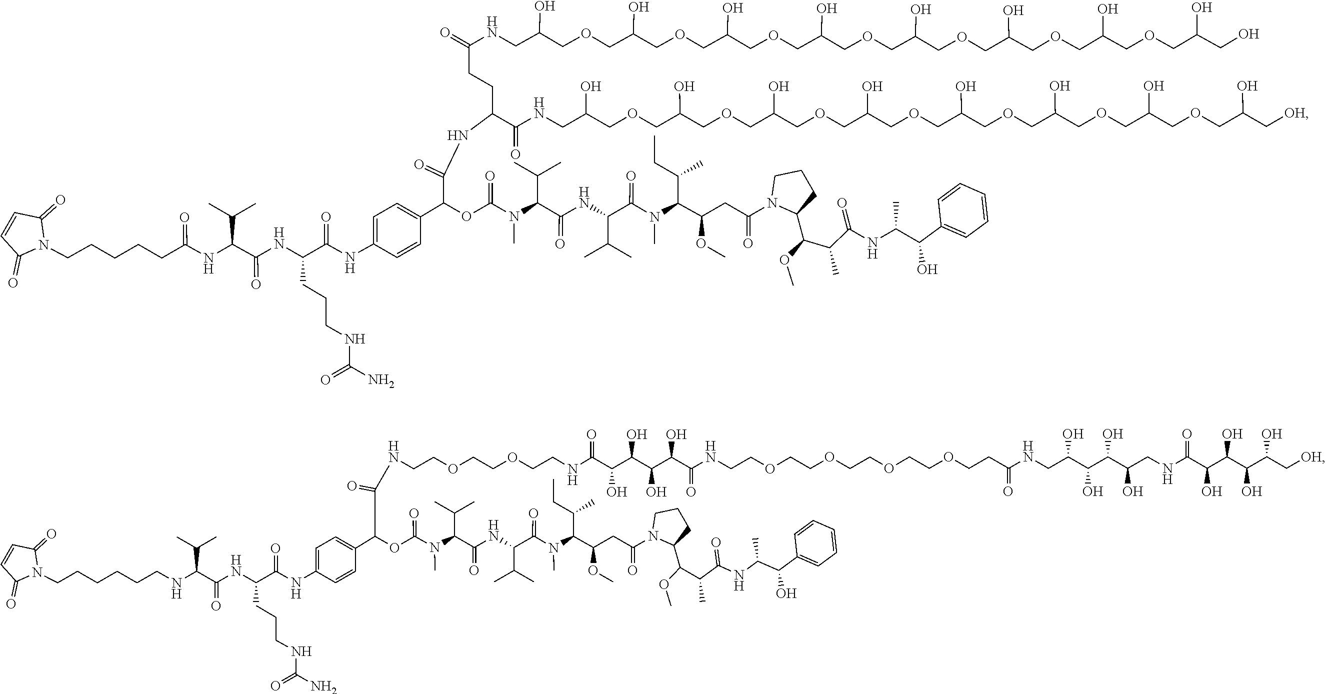

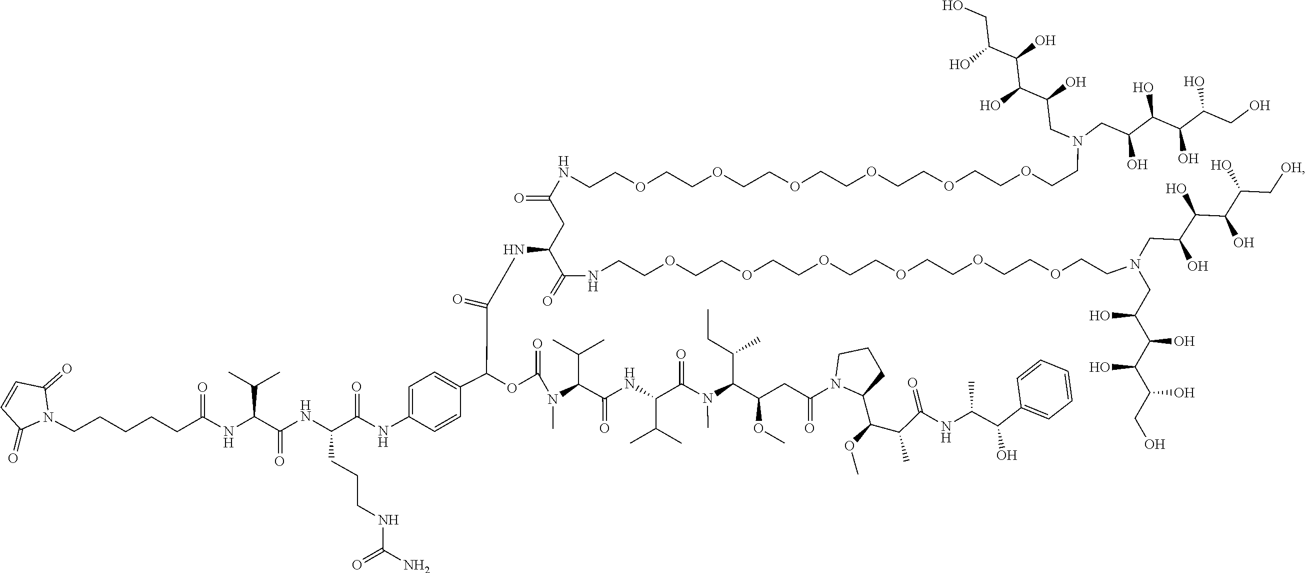 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@@H](CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

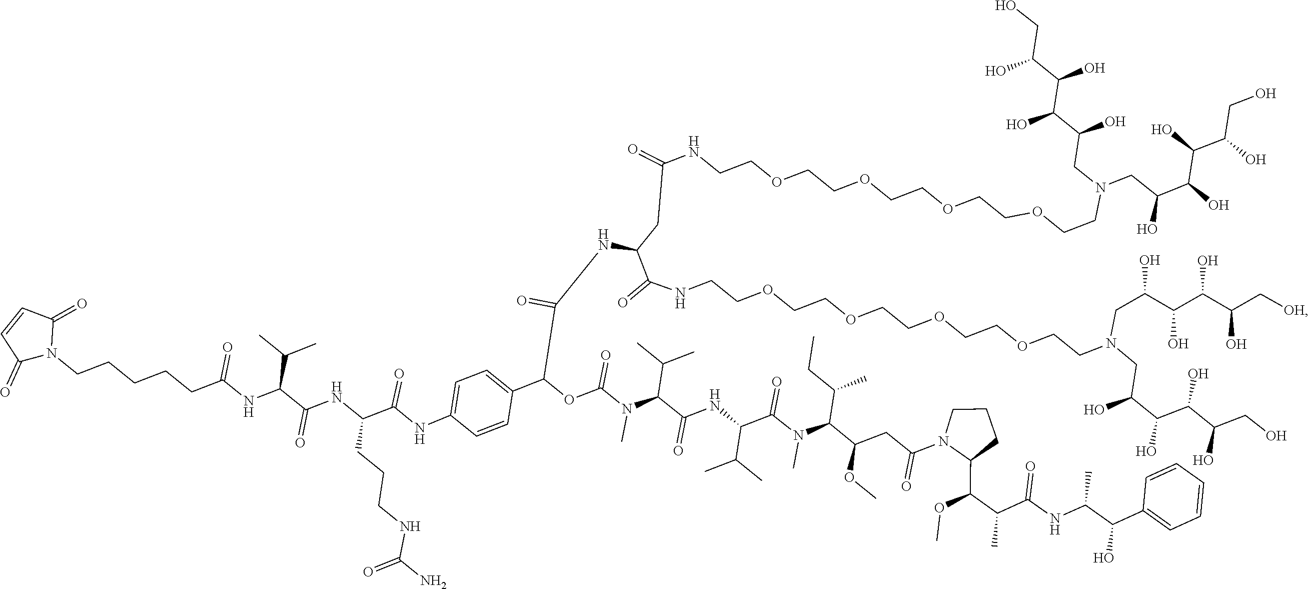 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@@H](CC(=O)NCCOCCOCCOCCOCCN(C[C@H](O)[C@H](O)[C@@H](O)[C@@H](O)CO)C[C@H](O)[C@@H](O)[C@H](O)[C@H](O)CO)C(=O)NCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

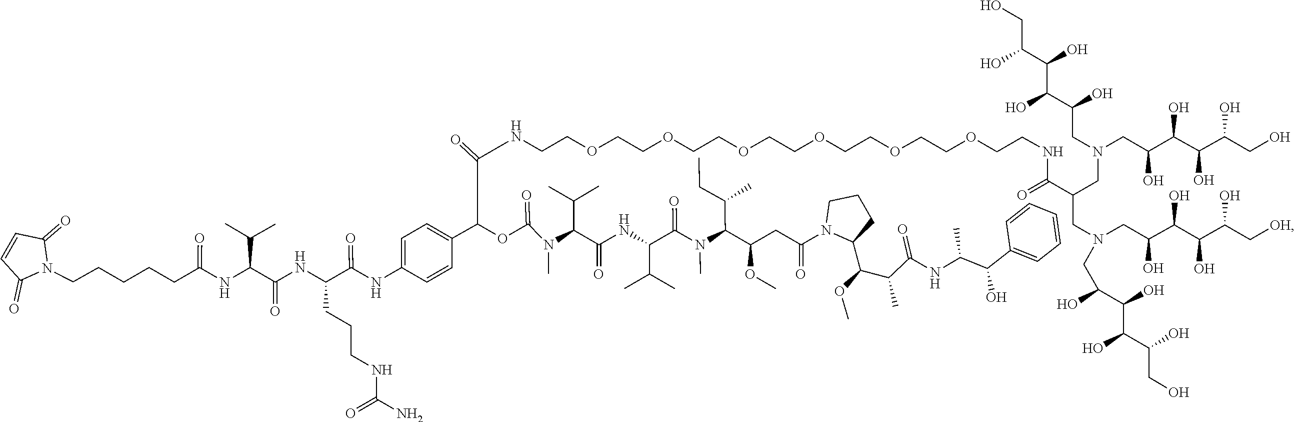 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCCOCCOCCOCCOCCOCCOCCNC(=O)C(CN(C[C@H](O)[C@@H](O)[C@H](O)[C@H](O)CO)C[C@H](O)[C@@H](O)[C@H](O)[C@H](O)CO)CN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

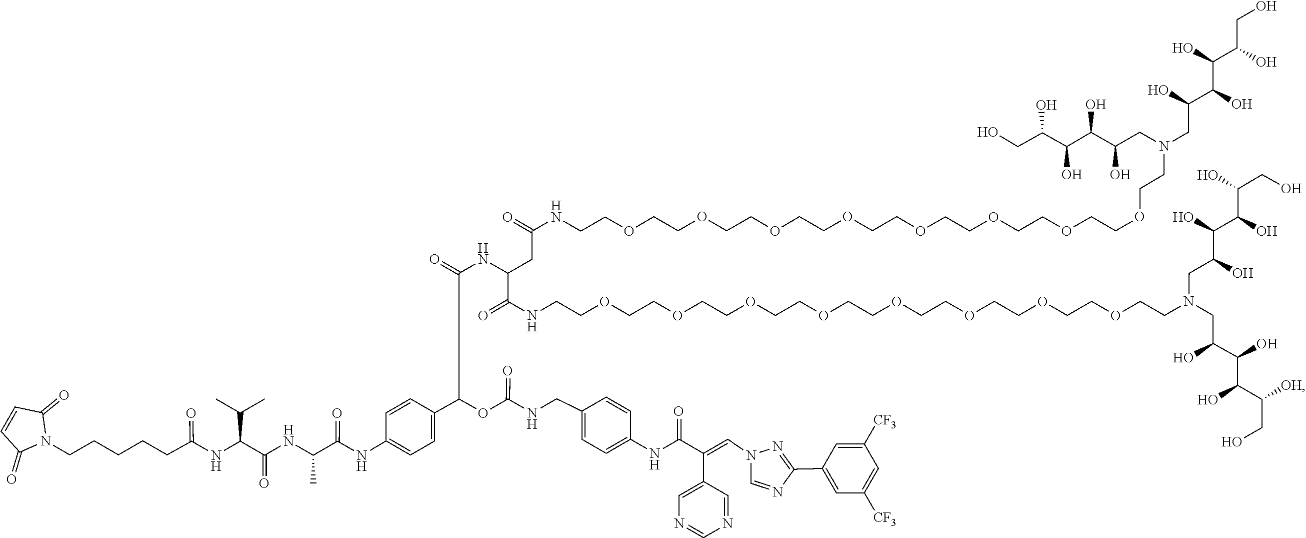 [SMILES: CC(C)[C@H](NC(=O)CCCCCN1C(=O)C=CC1=O)C(=O)N[C@@H](C)C(=O)Nc1ccc(C(OC(=O)NCc2ccc(NC(=O)/C(=C/n3cnc(-c4cc(C(F)(F)F)cc(C(F)(F)F)c4)n3)c3cncnc3)cc2)C(=O)NC(CC(=O)NCCOCCOCCOCCOCCOCCOCCOCCOCCN(C[C@@H](O)[C@H](O)[C@@H](O)[C@@H](O)CO)C[C@@H](O)[C@H](O)[C@@H](O)[C@@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

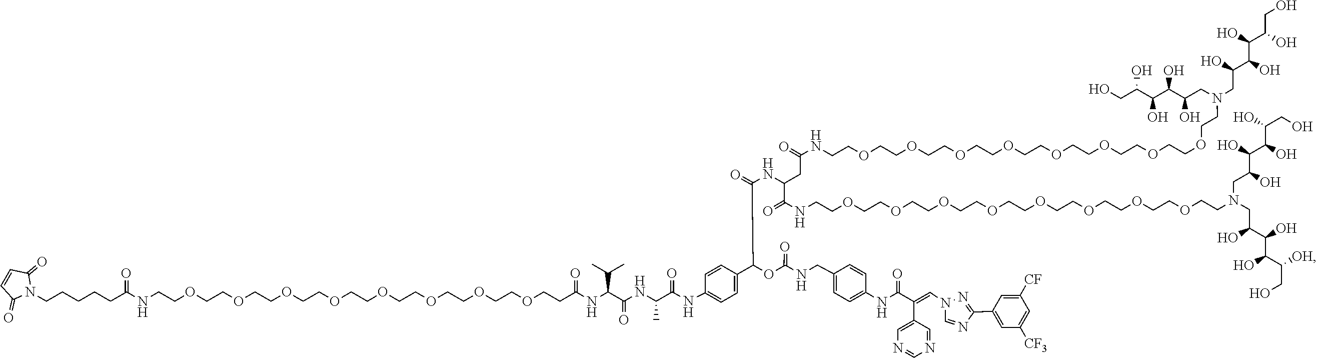

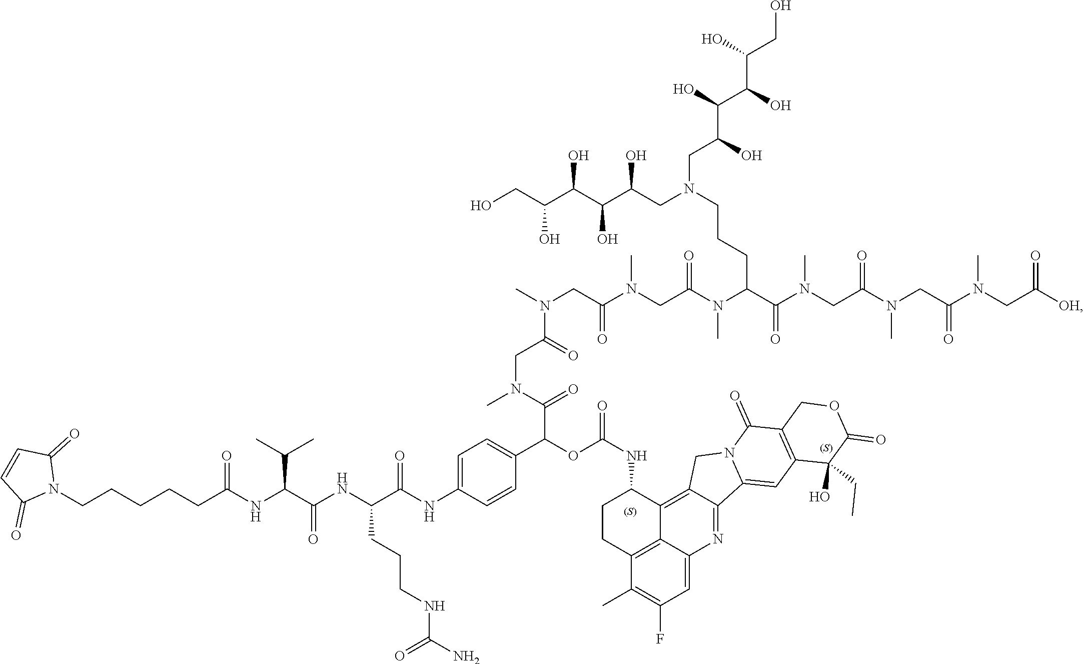

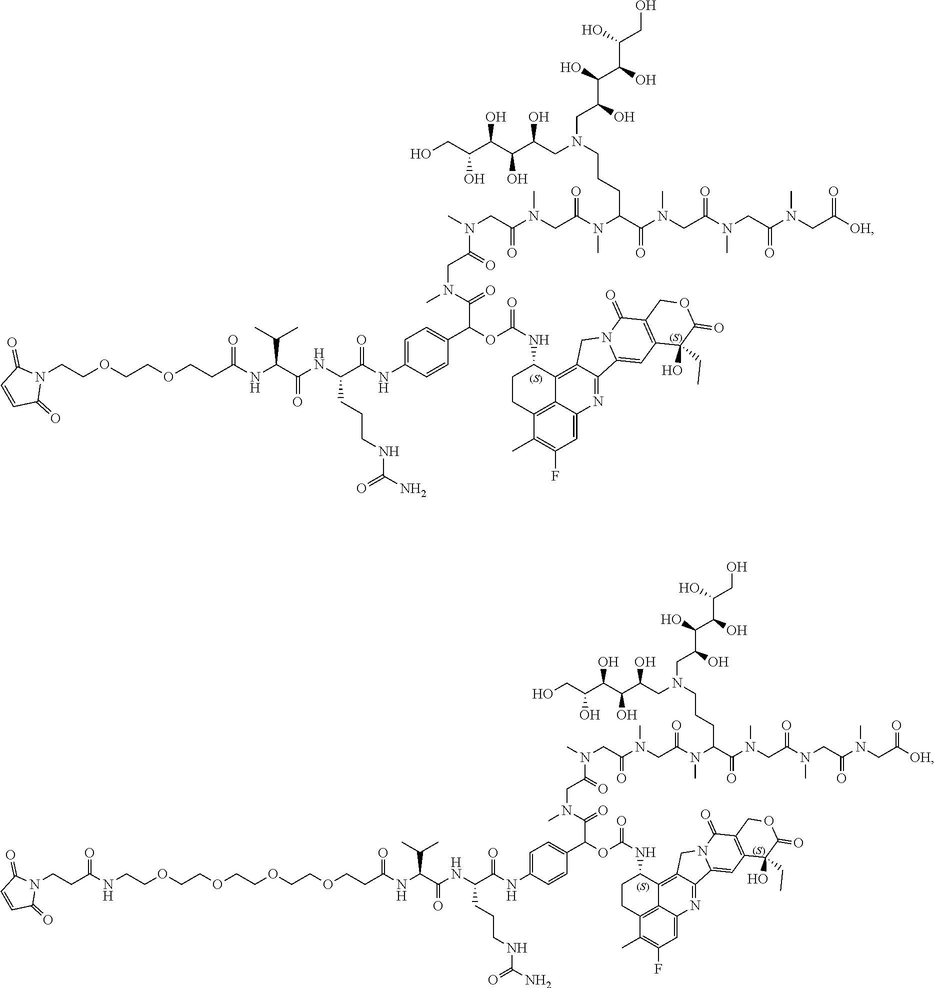

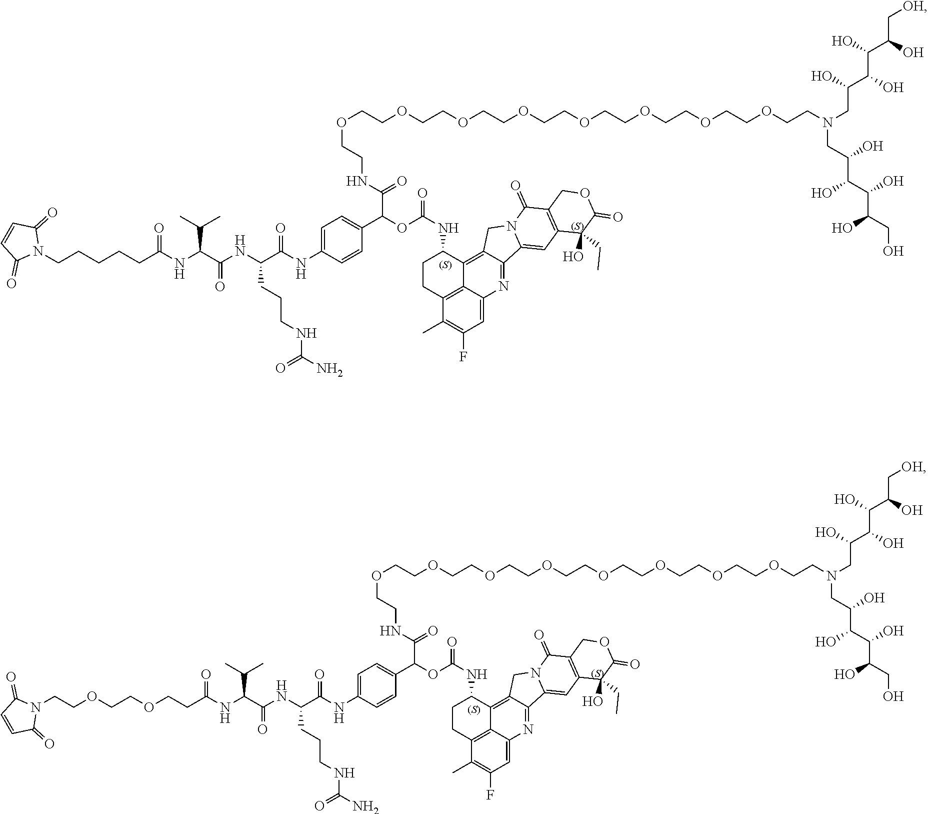

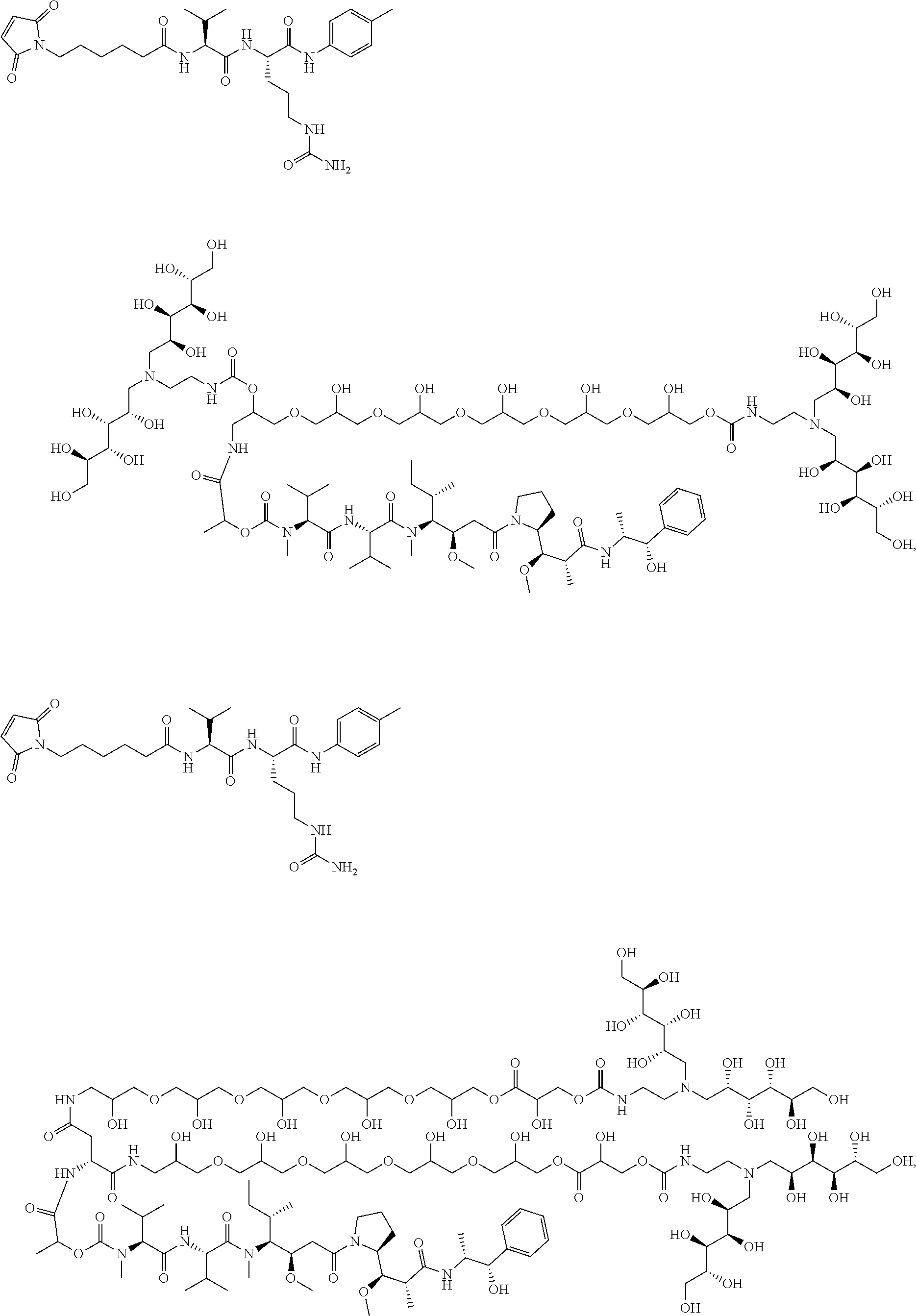

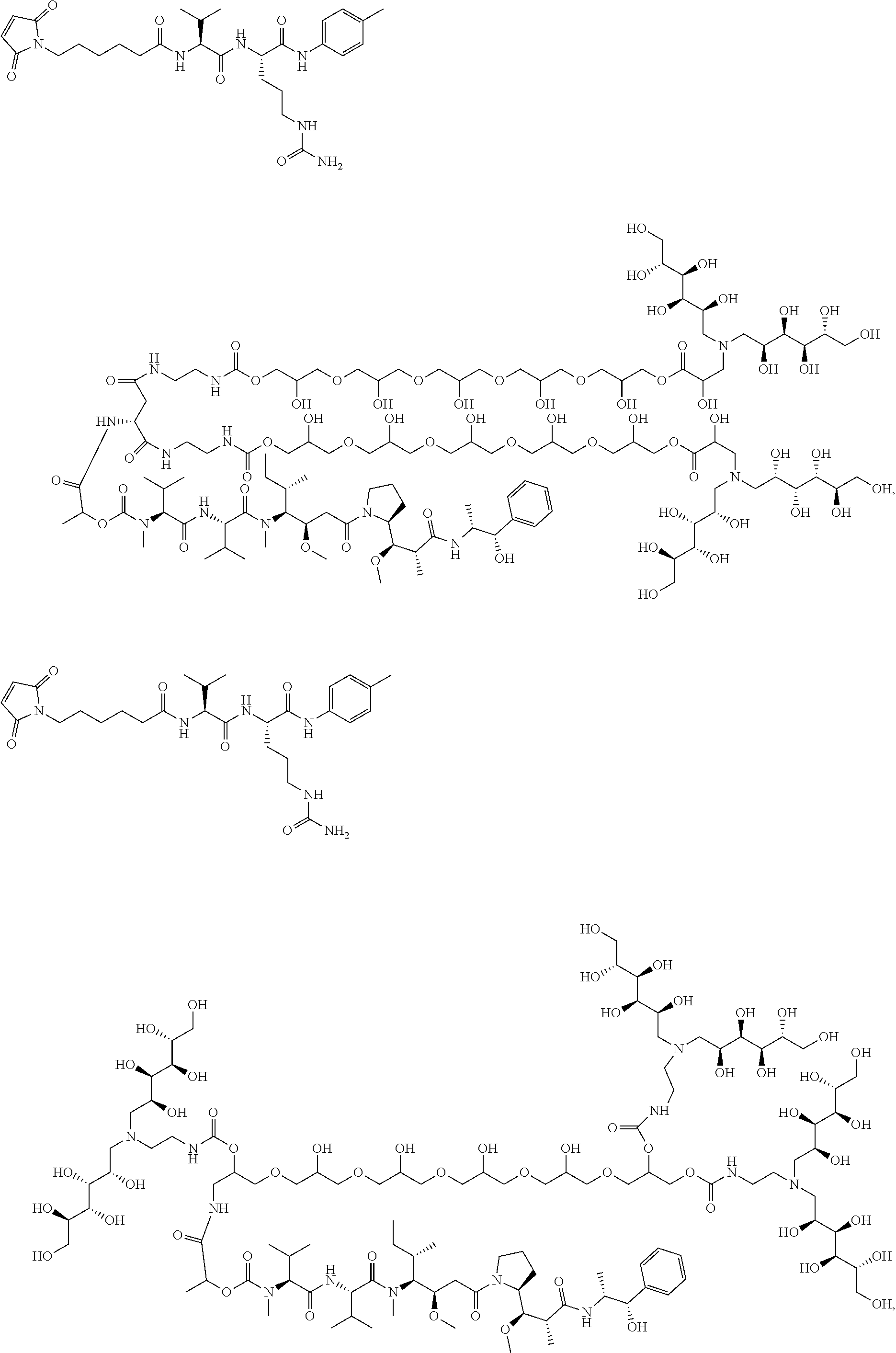

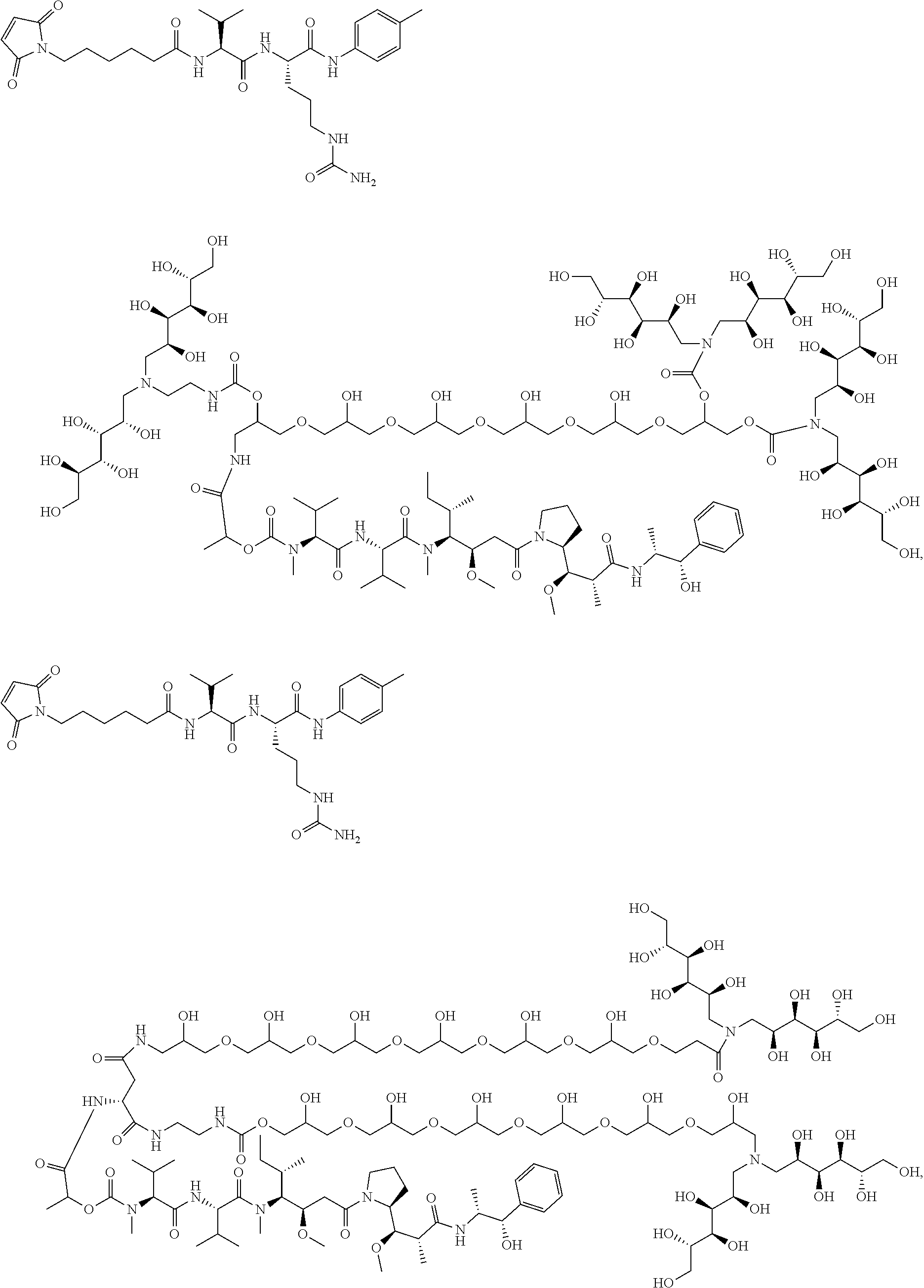

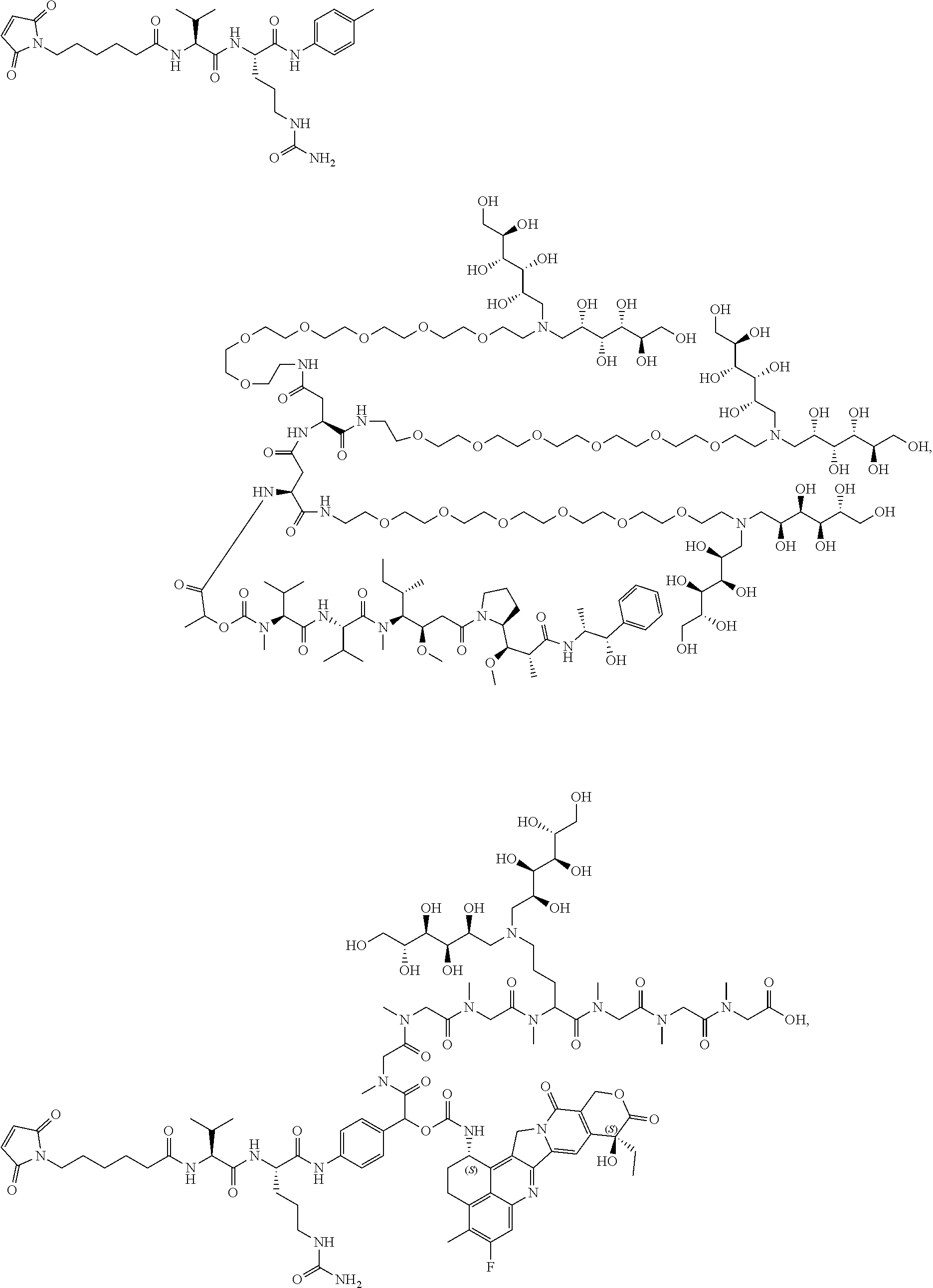

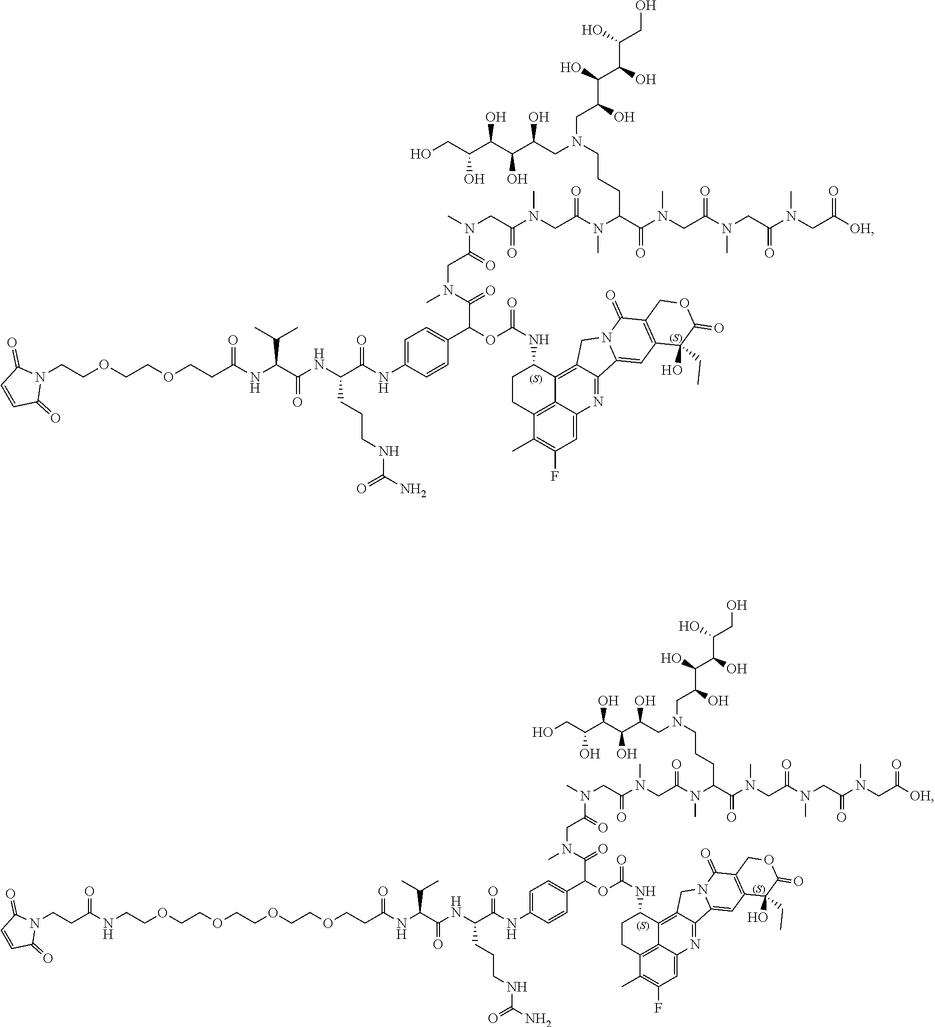

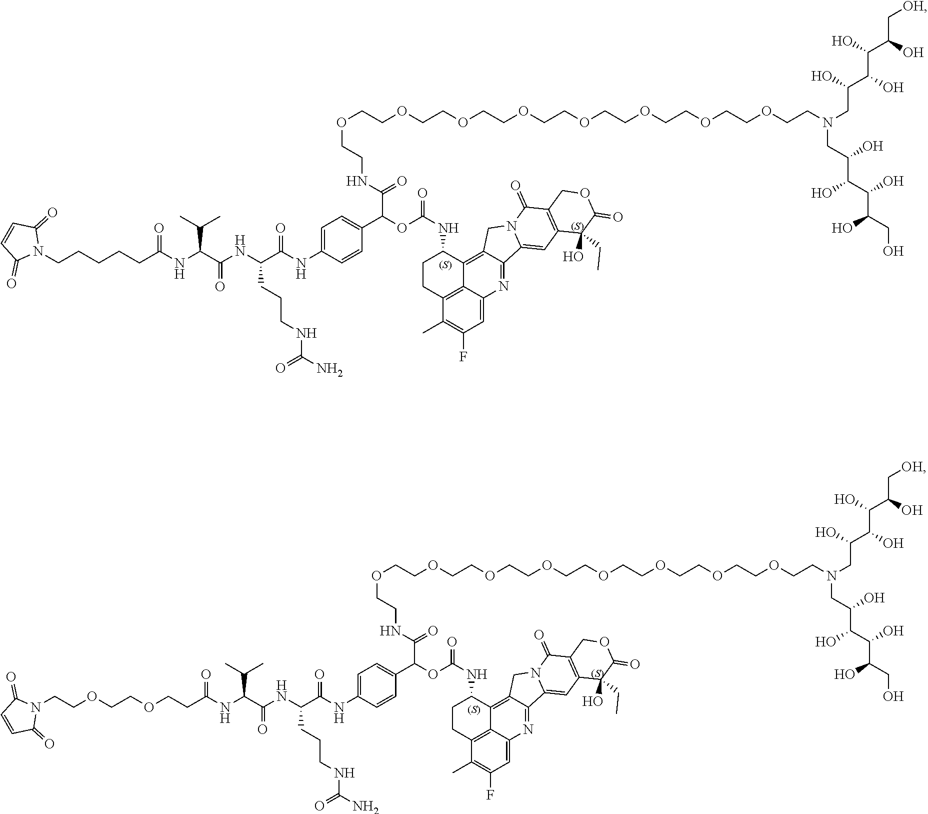

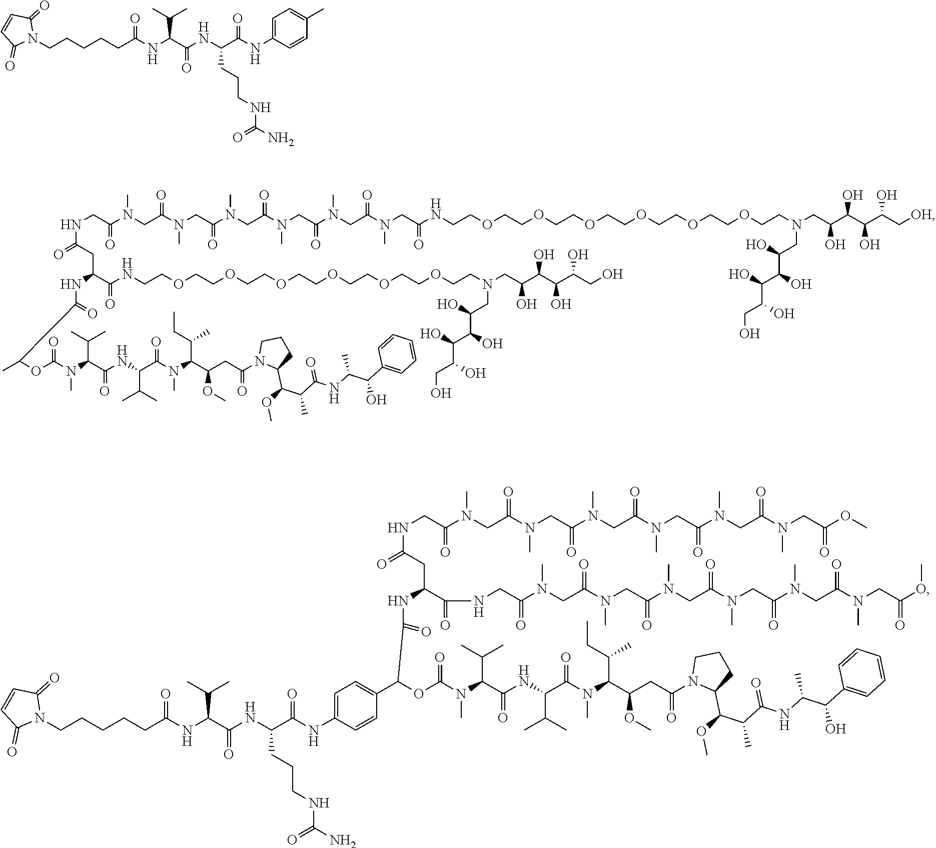

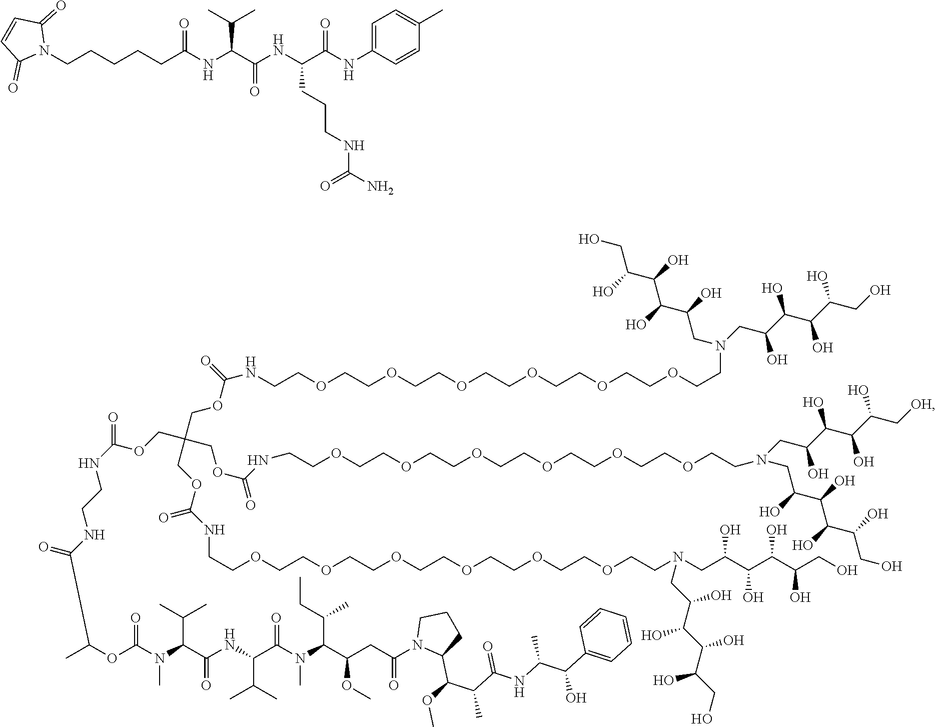

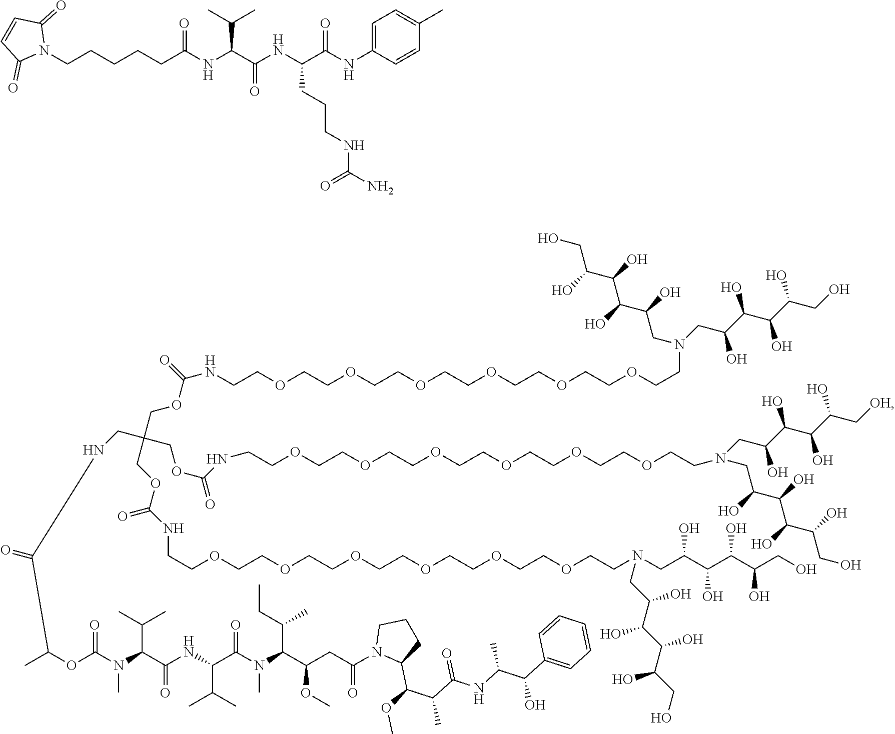

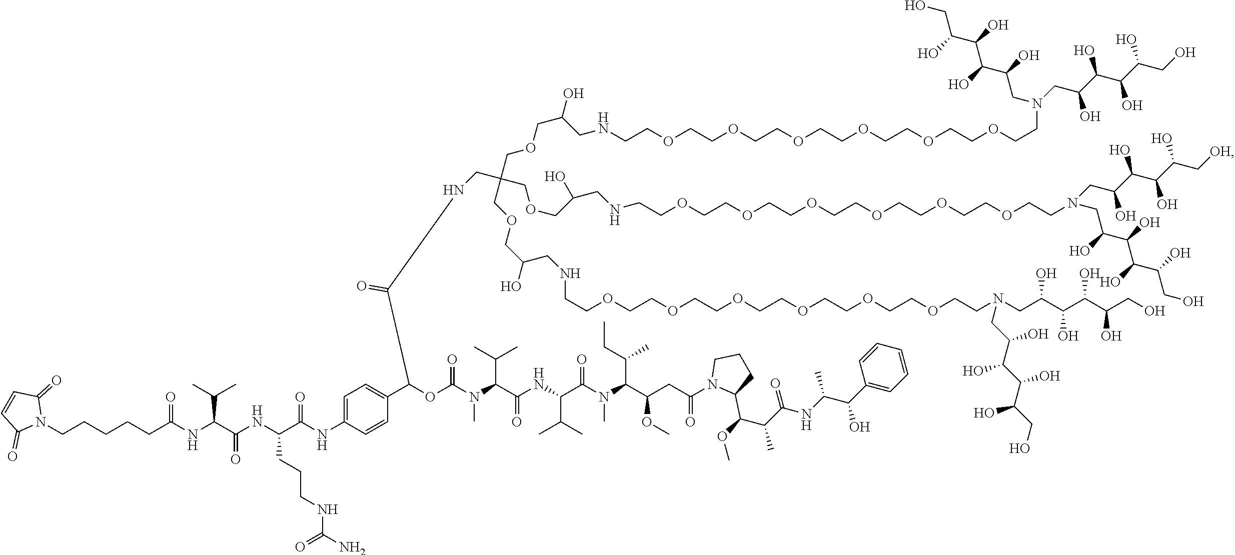

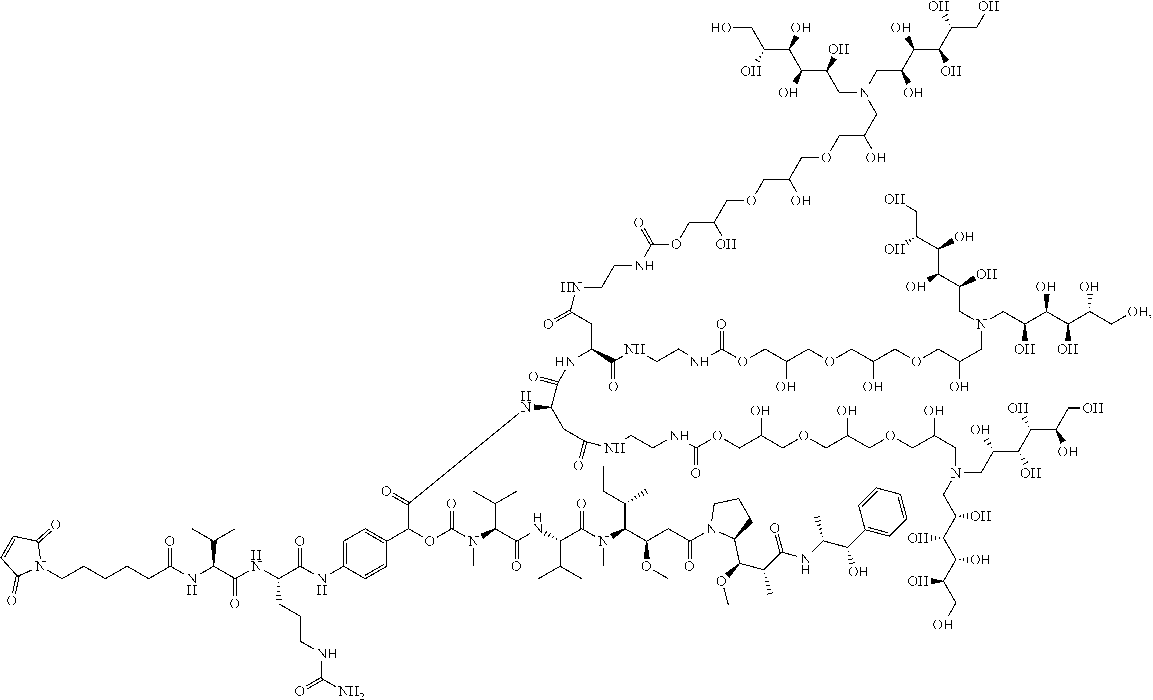

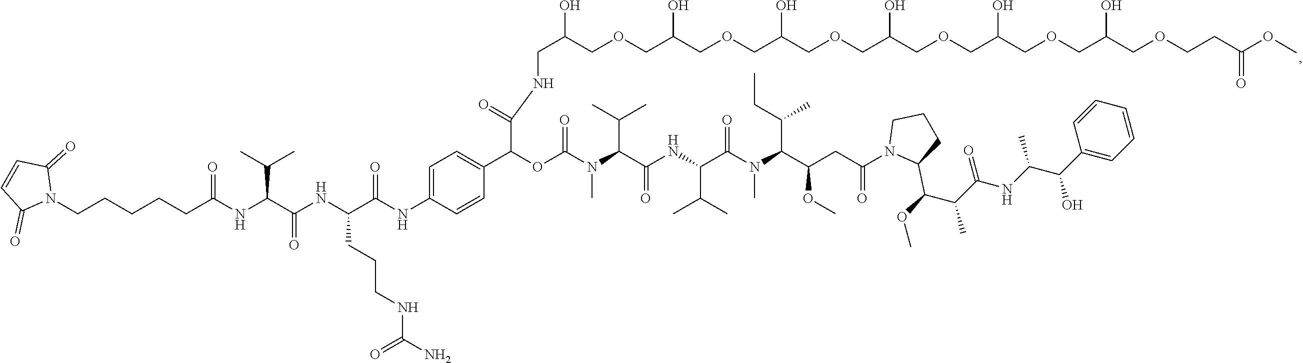 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCC(O)COCC(O)COCC(O)COCC(O)COCC(O)COCC(O)COCCC(=O)OC)c1ccc(NC(=O)[C@H](CCCNC(N)=O)NC(=O)[C@@H](NC(=O)CCCCCN2C(=O)C=CC2=O)C(C)C)cc1)C(C)C]

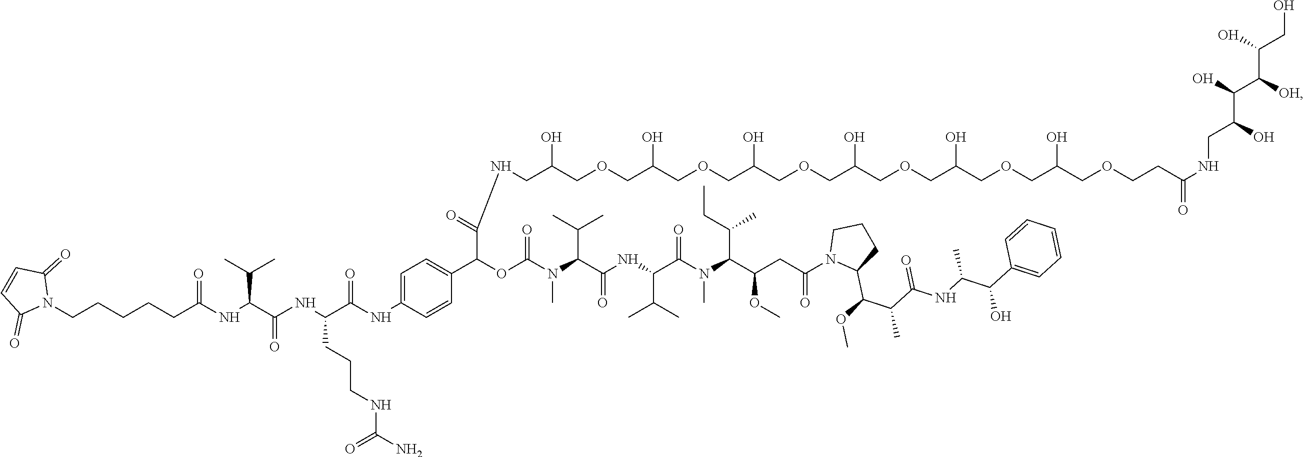 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCC(O)COCC(O)COCC(O)COCC(O)COCC(O)COCC(O)COCCC(=O)NC[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

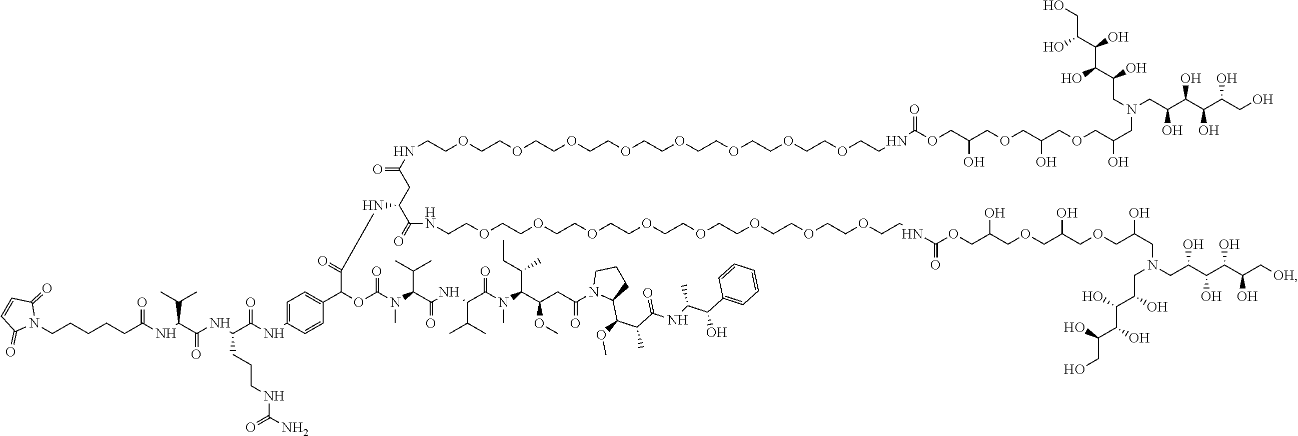

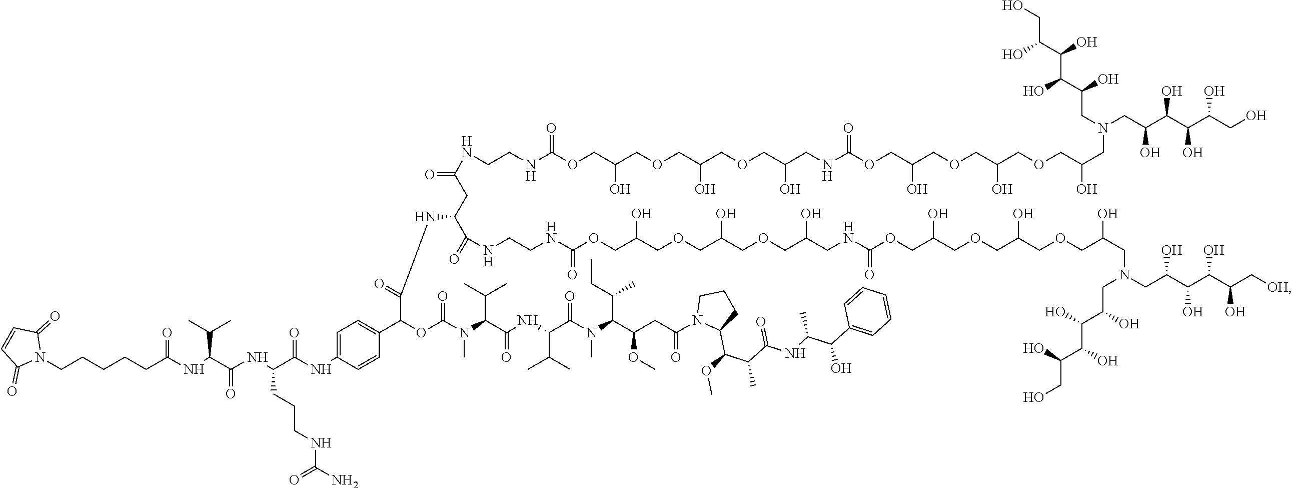

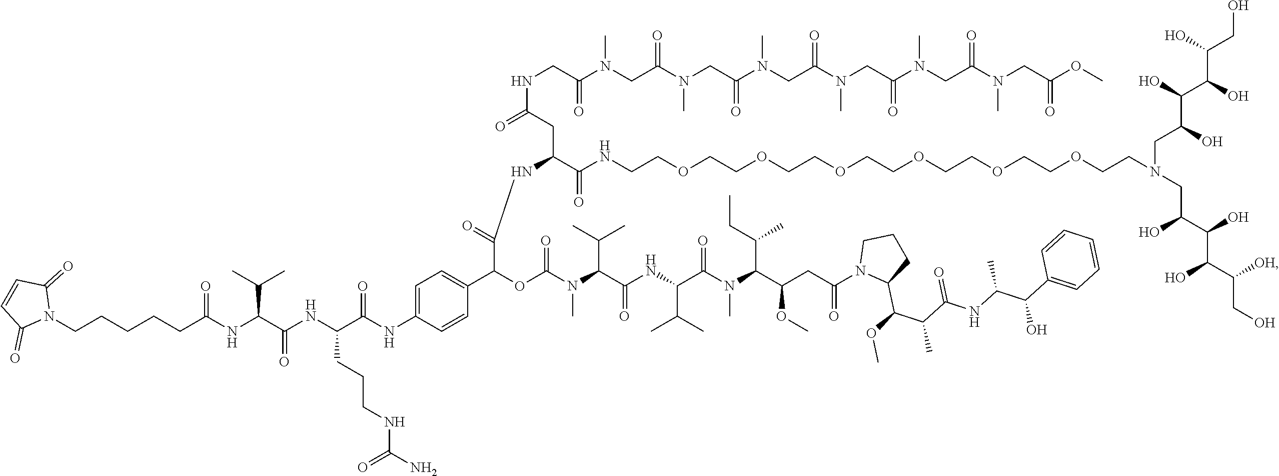 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@@H](CC(=O)NCC(=O)N(C)CC(=O)N(C)CC(=O)N(C)CC(=O)N(C)CC(=O)N(C)CC(=O)N(C)CC(=O)OC)C(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

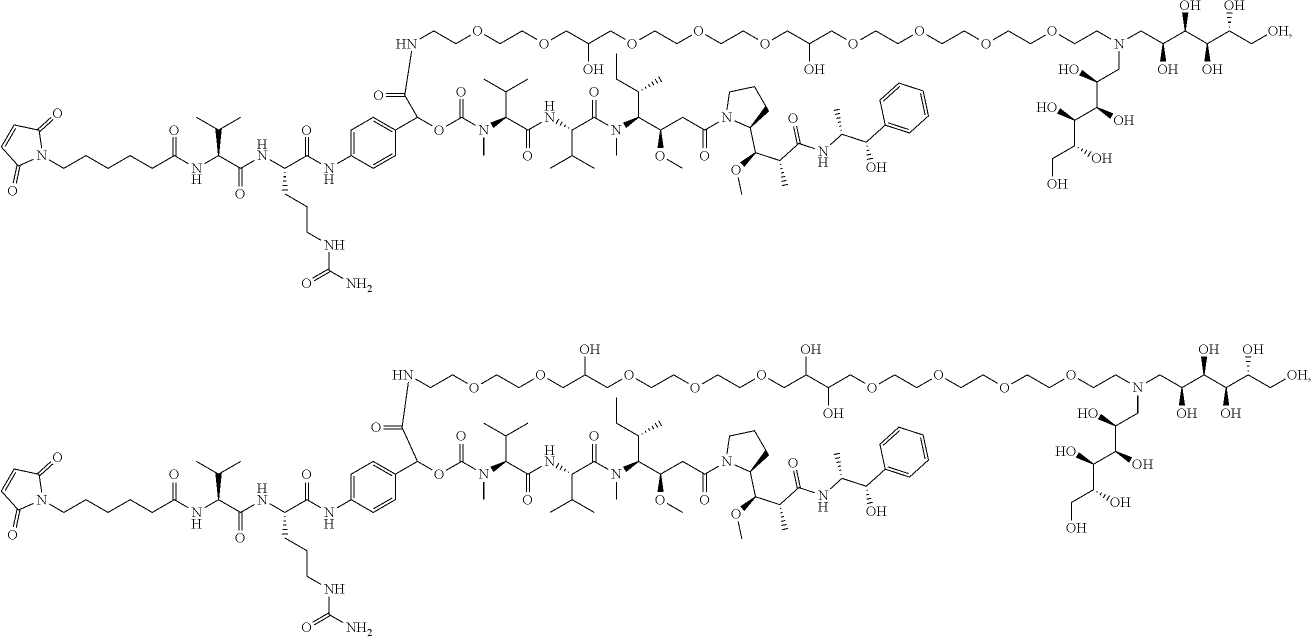

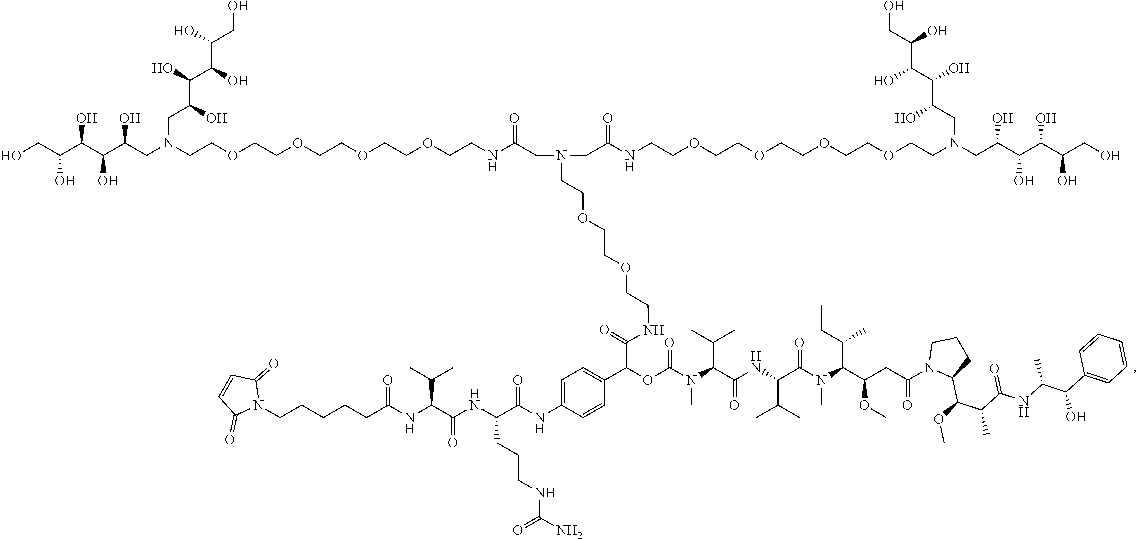 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCCOCCOCCN(CC(=O)NCCOCCOCCOCCOCCN(C[C@H](O)[C@@H](O)[C@H](O)[C@H](O)CO)C[C@H](O)[C@@H](O)[C@H](O)[C@H](O)CO)CC(=O)NCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

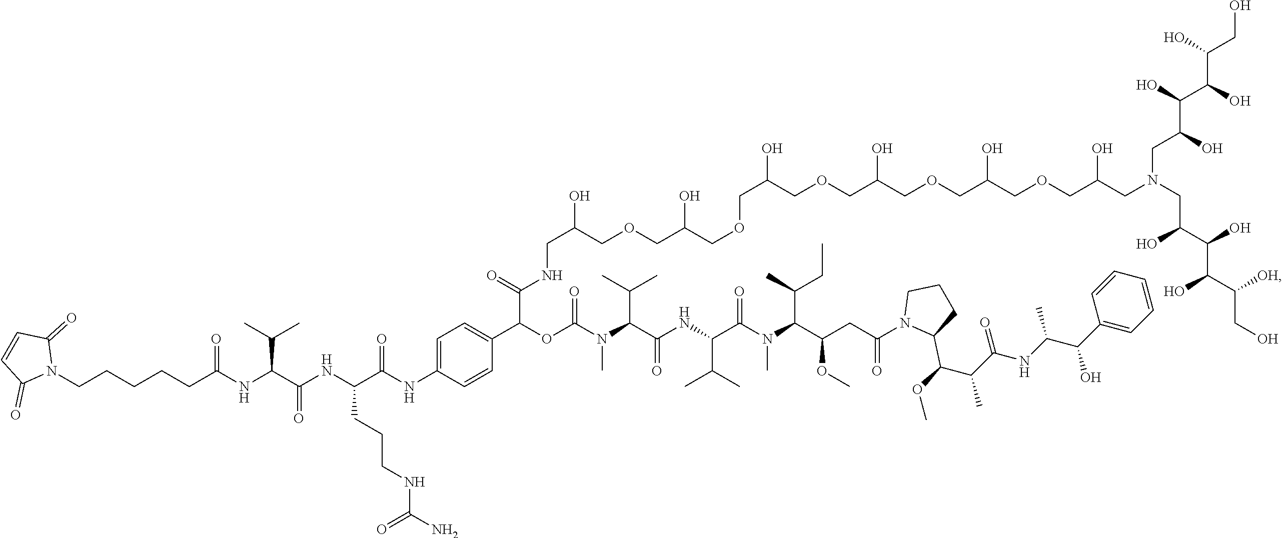 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

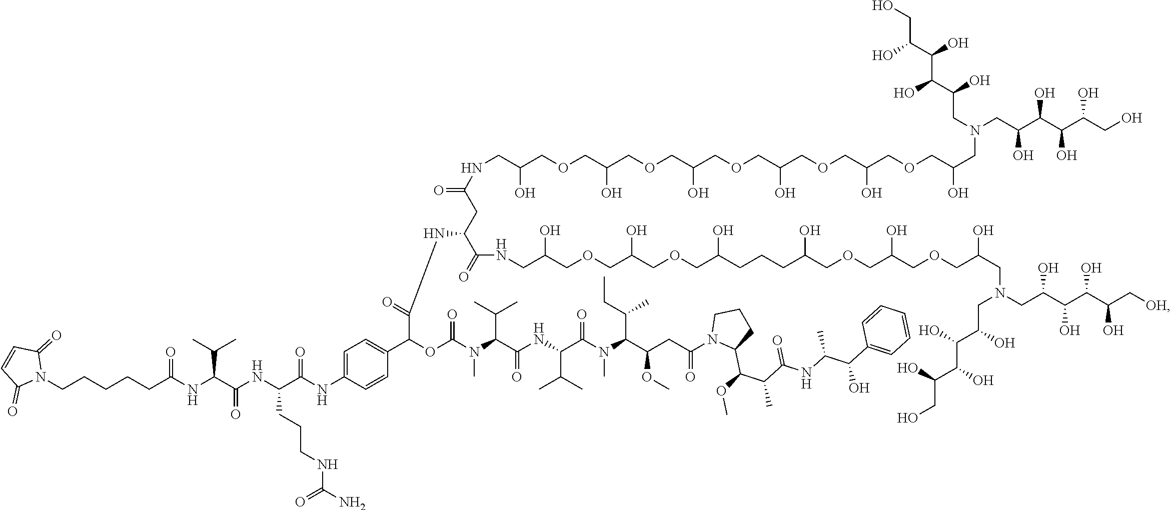 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@H](CC(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCC(O)COCC(O)COCC(O)CCCC(O)COCC(O)COCC(O)CN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

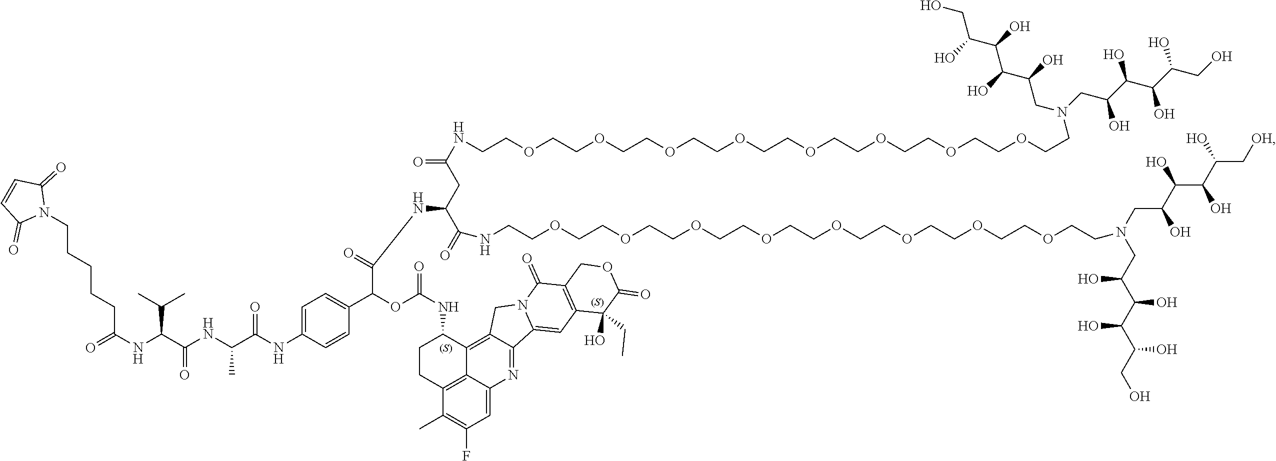

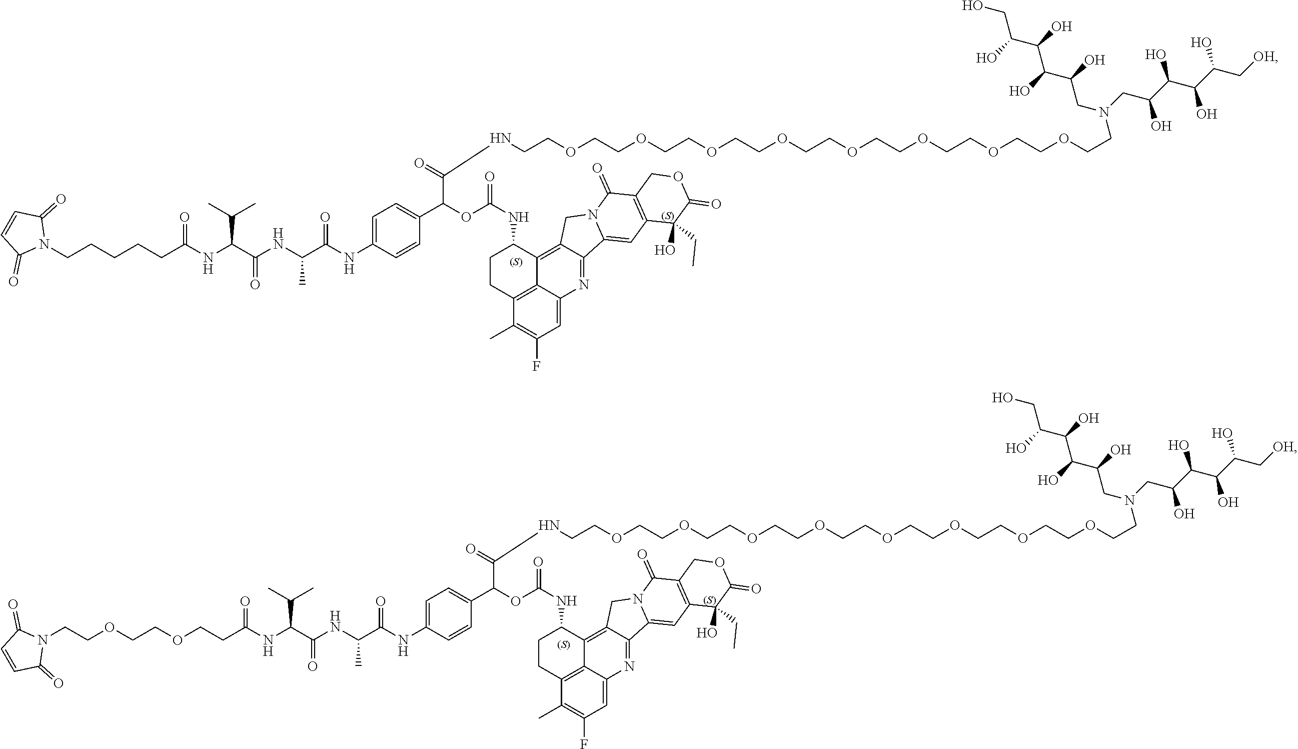

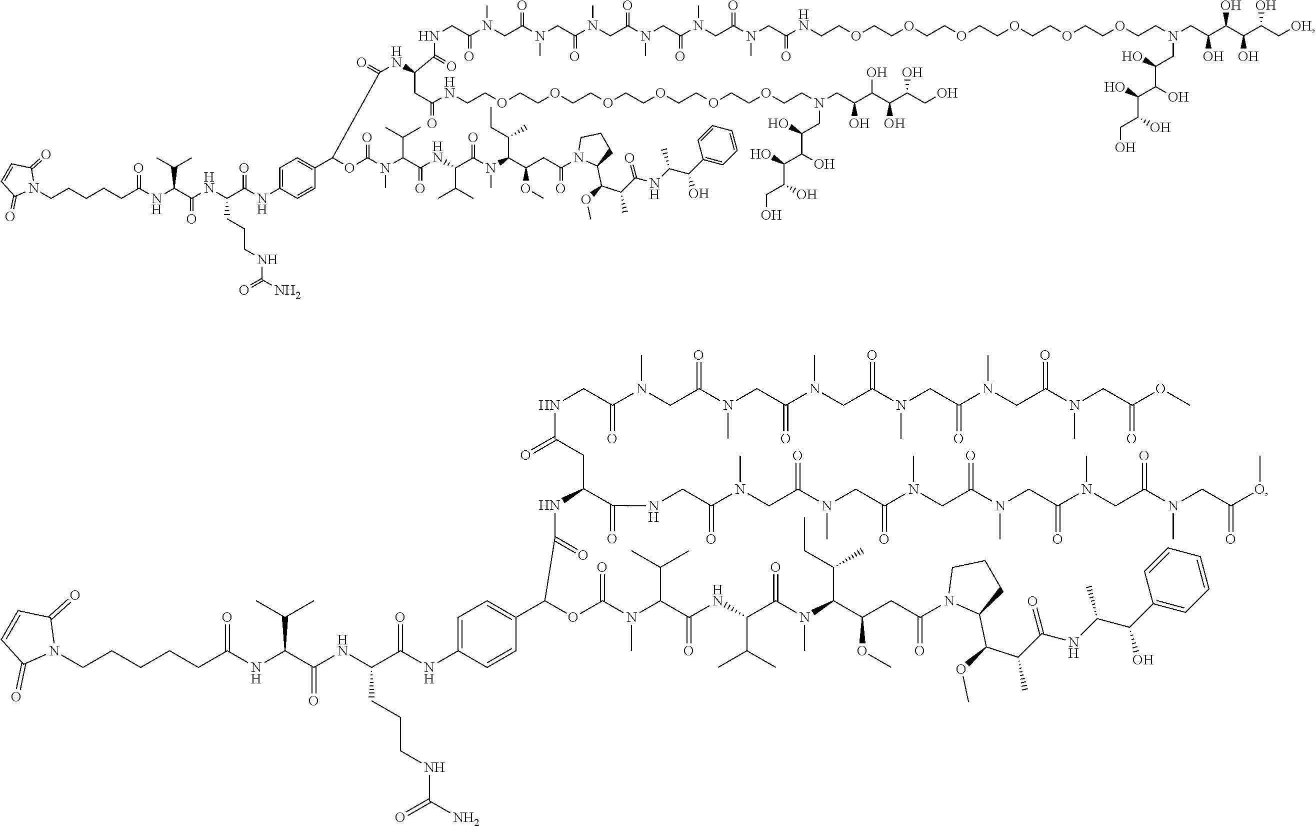

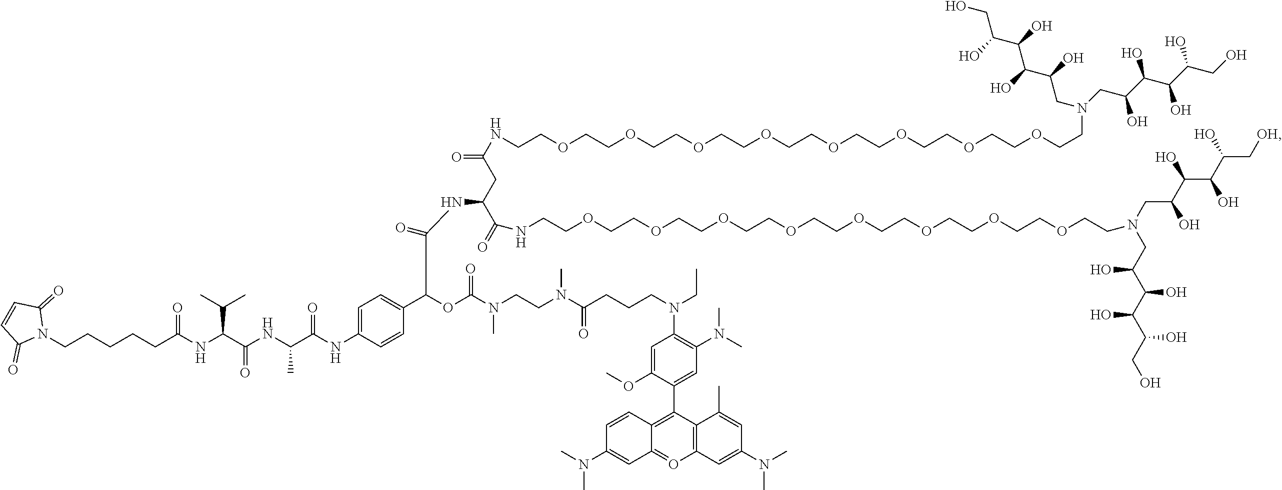

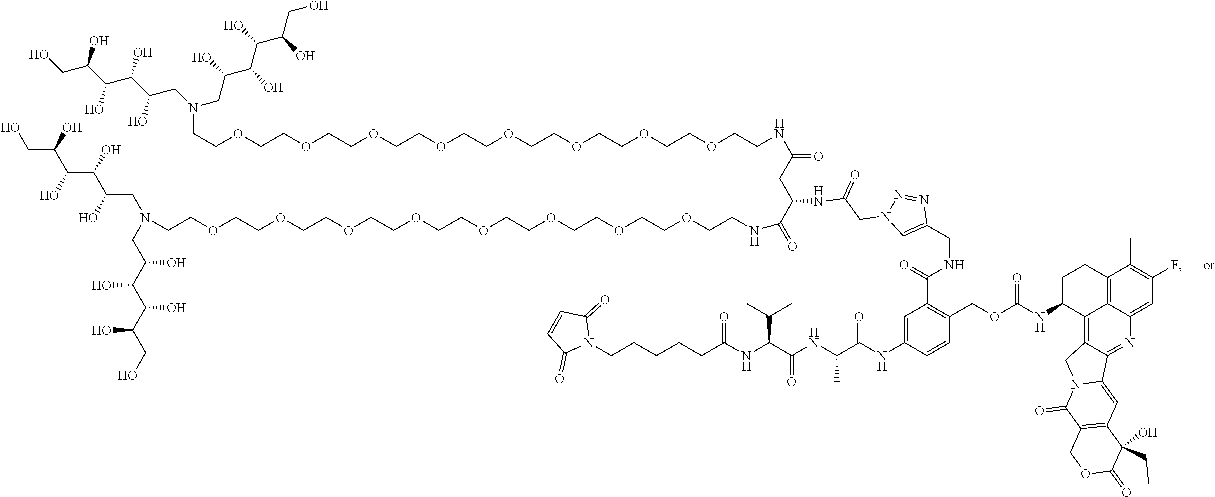 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)CCCCCN2C(=O)C=CC2=O)C(C)C)cc1C(=O)NCc1cn(CC(=O)N[C@@H](CC(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)nn1)CC3]

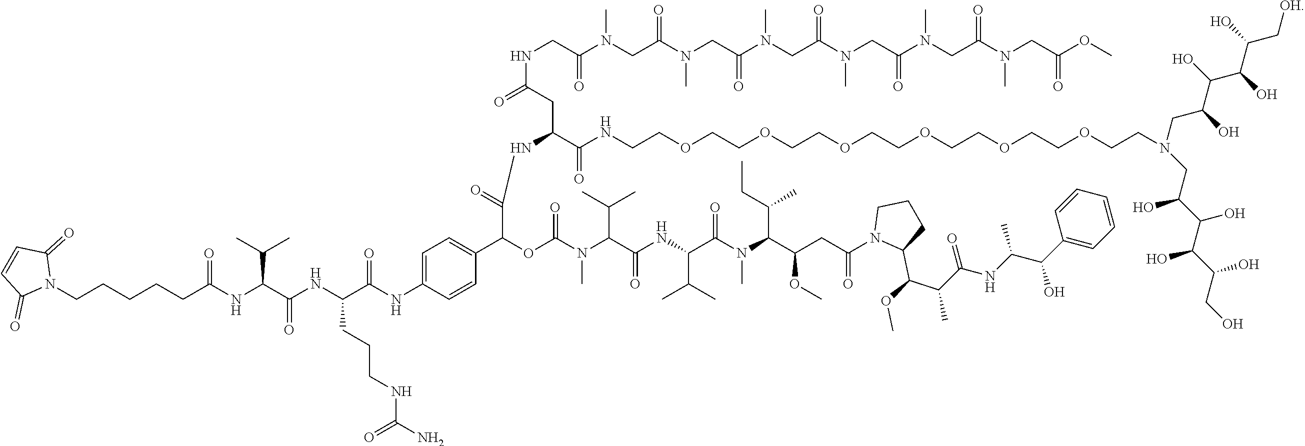 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)C(C(C)C)N(C)C(=O)OC(C(=O)N[C@@H](CC(=O)NCC(=O)N(C)CC(=O)N(C)CC(=O)N(C)CC(=O)N(C)CC(=O)N(C)CC(=O)N(C)CC(=O)OC)C(=O)NCCOCCOCCOCCOCCOCCOCCN(C[C@H](O)C(O)[C@H](O)[C@H](O)CO)C[C@H](O)C(O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

Embodiment 109. A conjugate comprising a Targeting group attached to the Drug-linker of any one of Embodiments 85 to 108, wherein the Targeting group specifically binds to the target molecule.

Embodiment 110. The conjugate of Embodiment 109, wherein the Targeting group is selected from an antibody or an antigen-binding portion thereof.

Embodiment 111. The conjugate of Embodiment 110, wherein the Targeting group is a monoclonal antibody, a Fab, a Fab′, an F(ab′), an Fv, a disulfide linked Fc, a scFv, a single domain antibody, a diabody, a bi-specific antibody, or a multi-specific antibody.

Embodiment 112. The conjugate of Embodiment 100, wherein the Targeting group is a diabody, a DART, an anticalin, an affibody, an avimer, a DARPin, or an adnectin.

Embodiment 113. The conjugate of any one of Embodiments 109 to 112, wherein the Targeting group is mono-specific.

Embodiment 114. The conjugate of any one of Embodiments 109 to 113, wherein the Targeting group is bivalent.

Embodiment 115. The conjugate of any one of Embodiments 109 to 112, wherein the Targeting group is bispecific.

Embodiment 116. The conjugate of any one of Embodiments 109 to 115, wherein the average drug loading (p^{load}) of the conjugate is from about 1 to about 8, about 2, about 4, about 6, about 8, about 10, about 12, about 14, about 16, about 3 to about 5, about 6 to about 8, or about 8 to about 16.

Embodiment 117. The conjugate of any one of Embodiments 109-116, selected from the following:

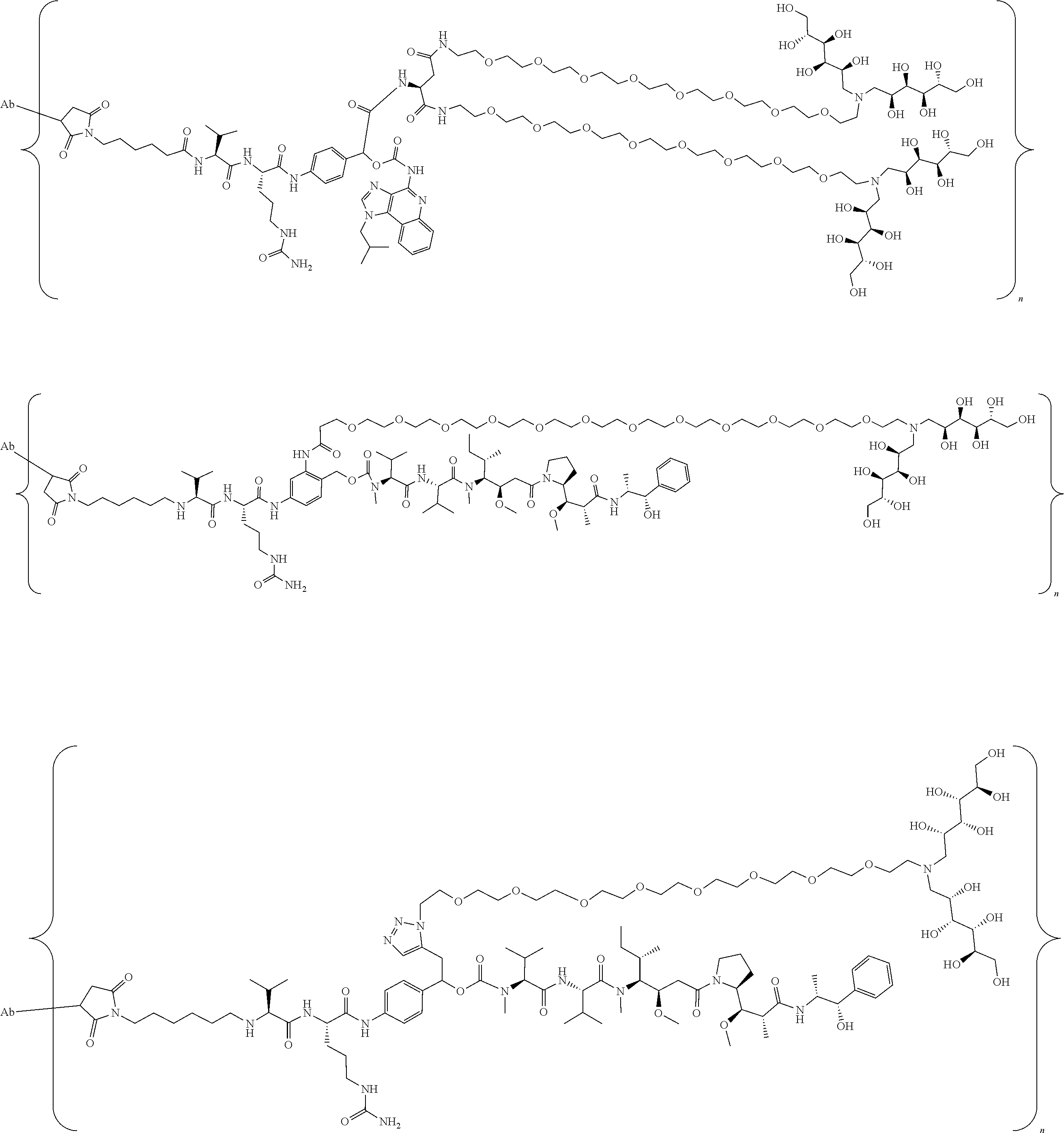

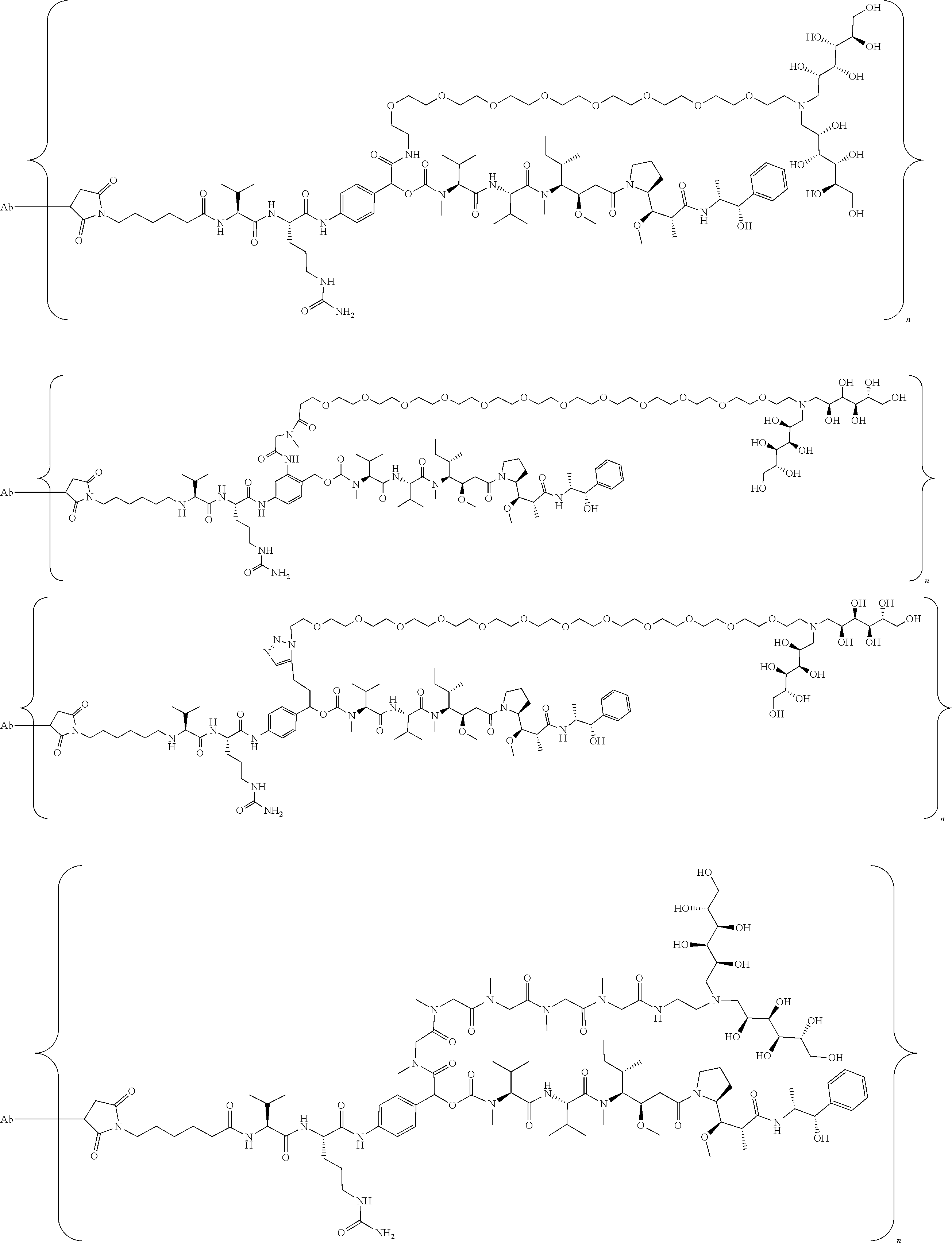

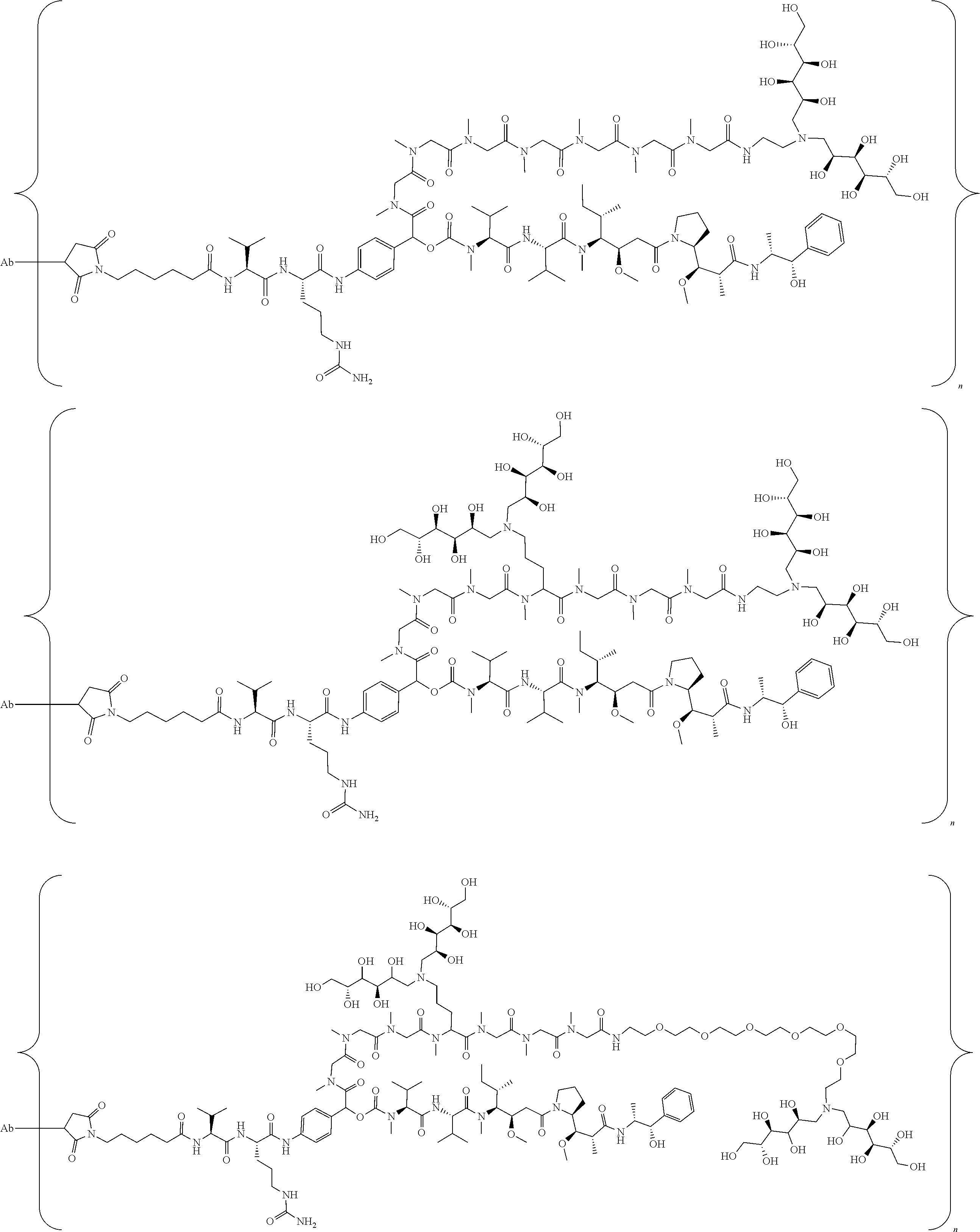

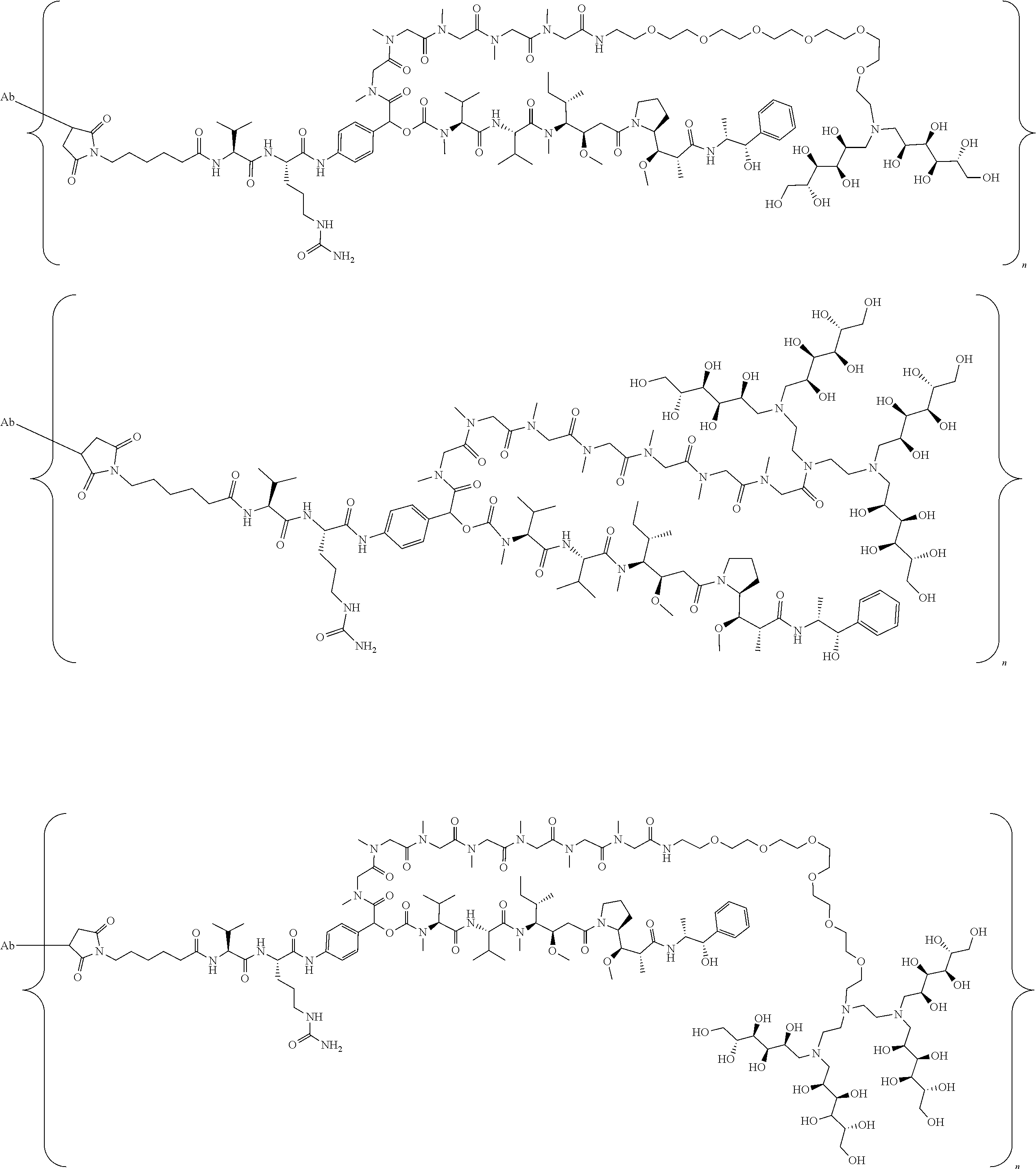

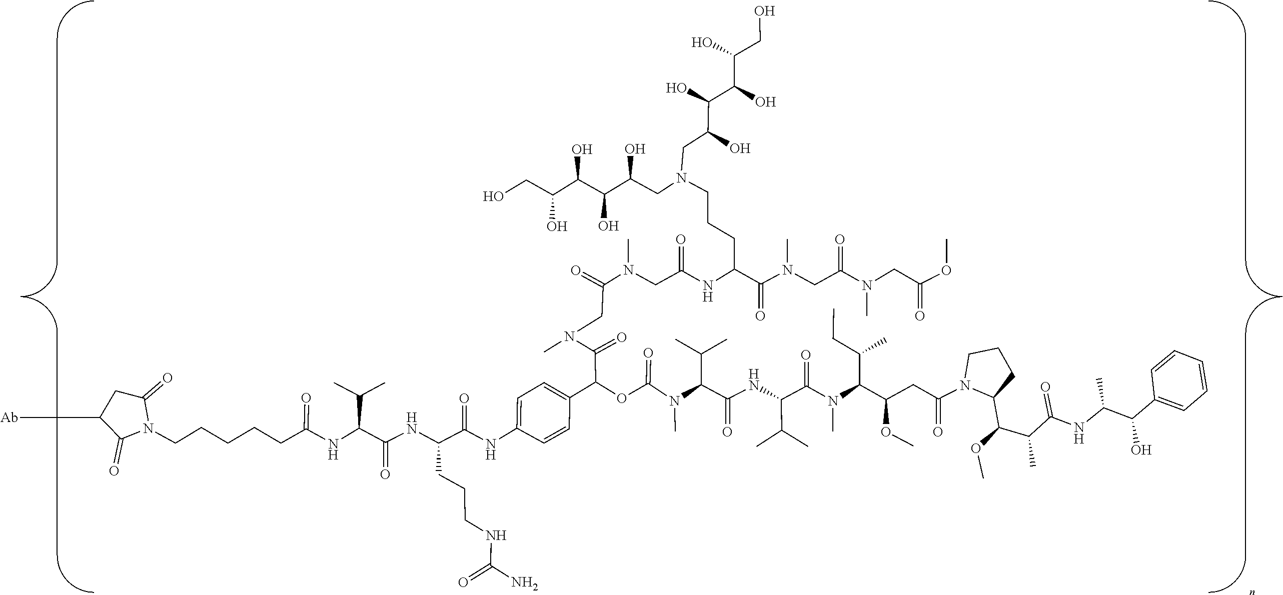

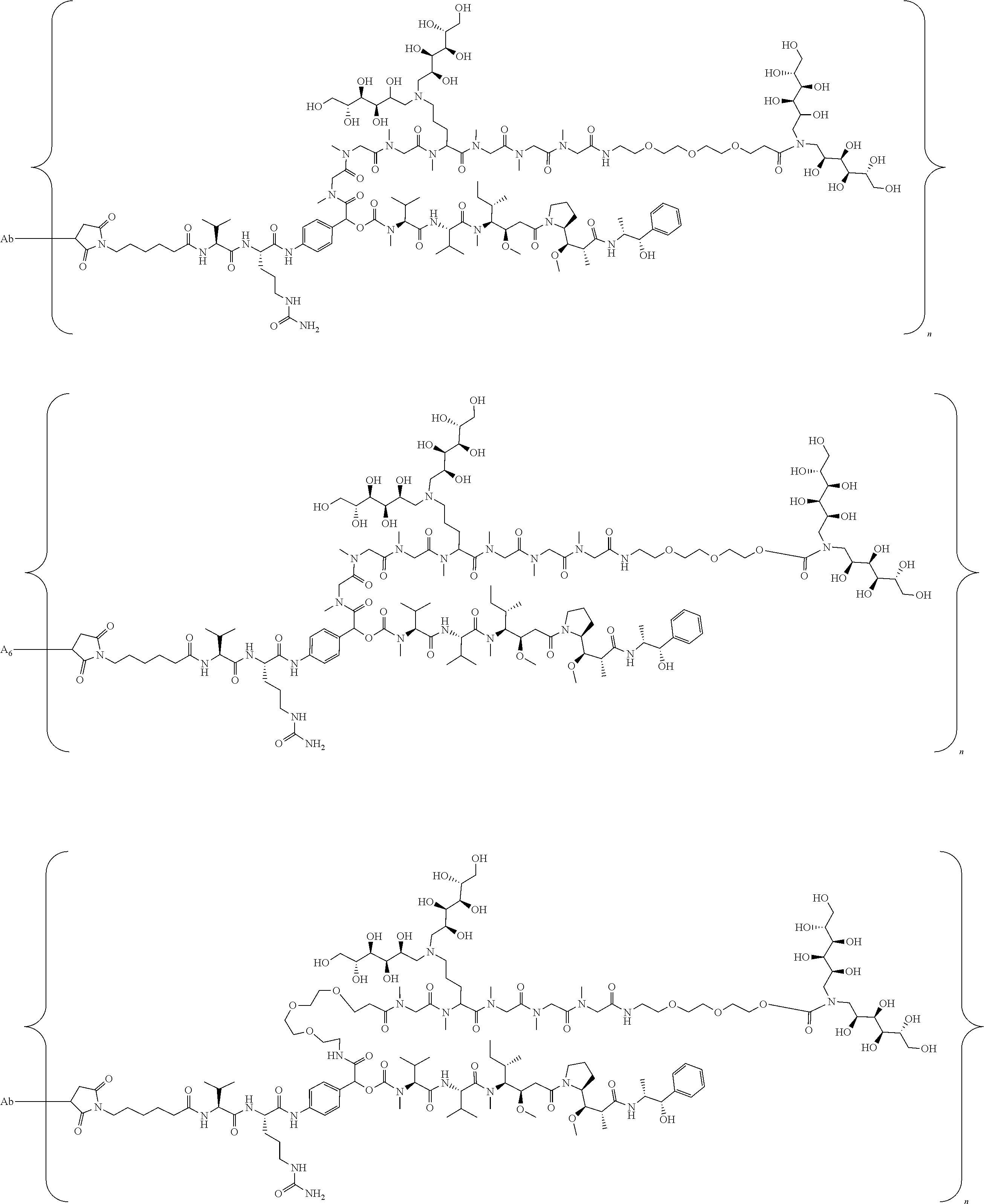

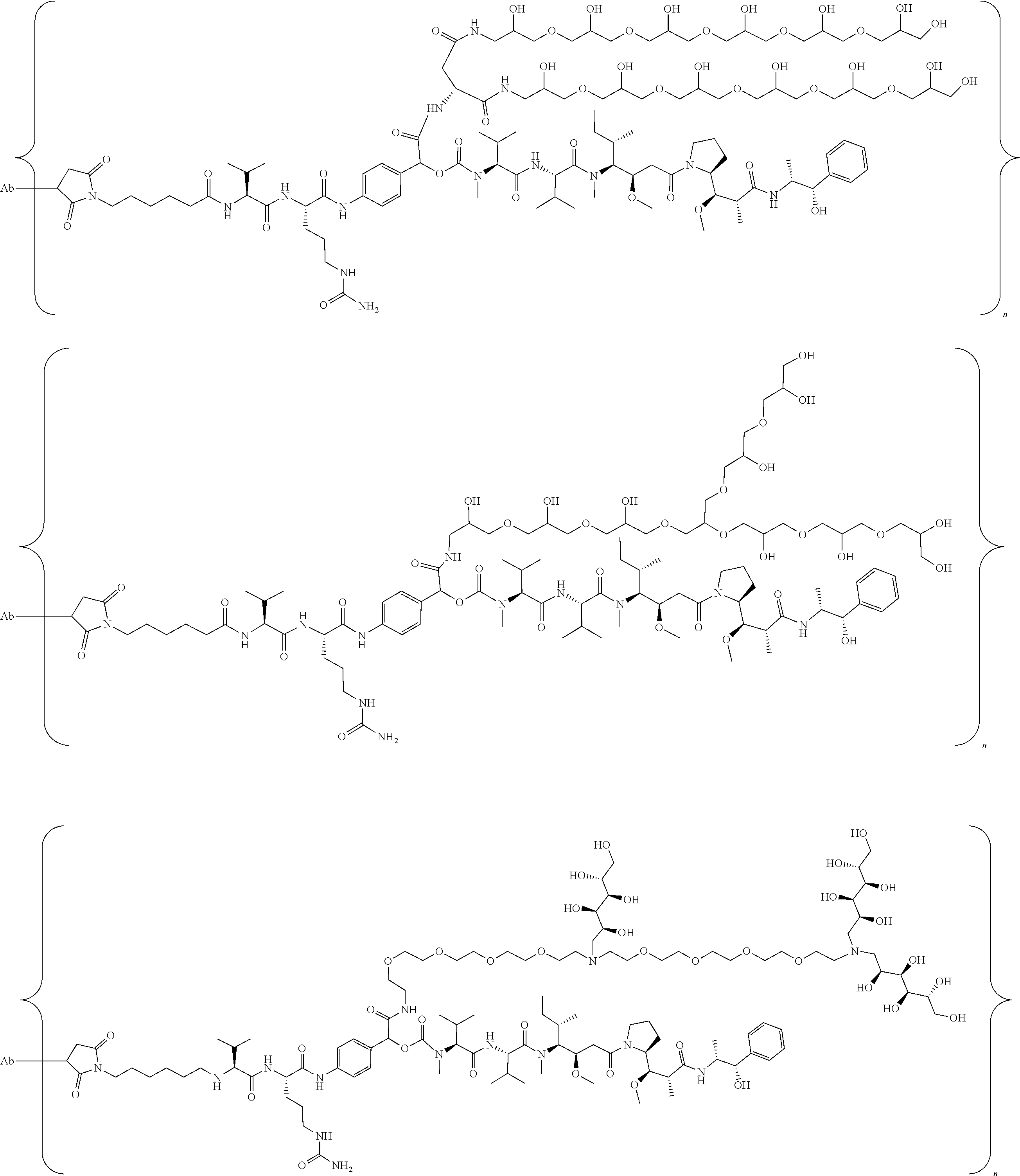

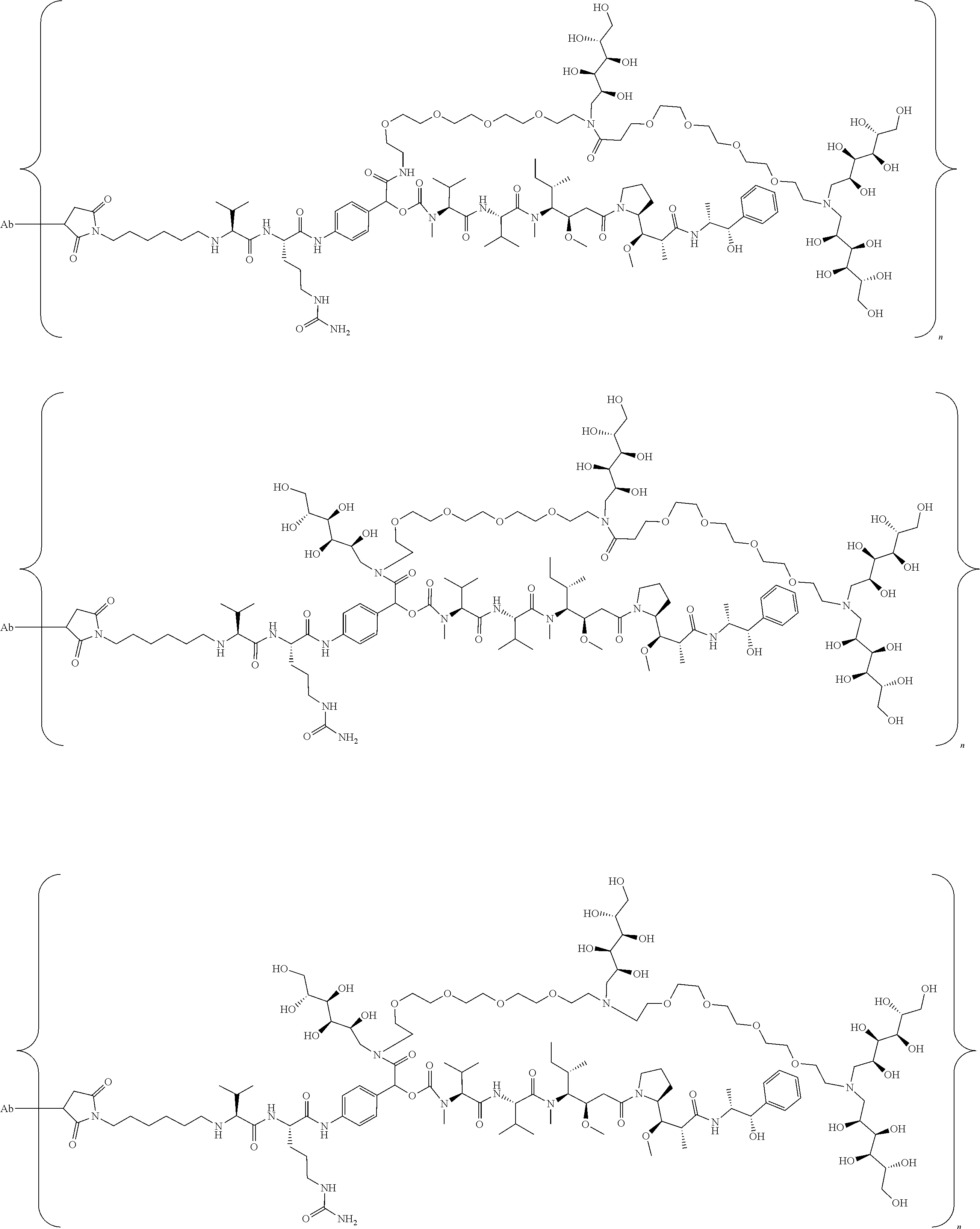

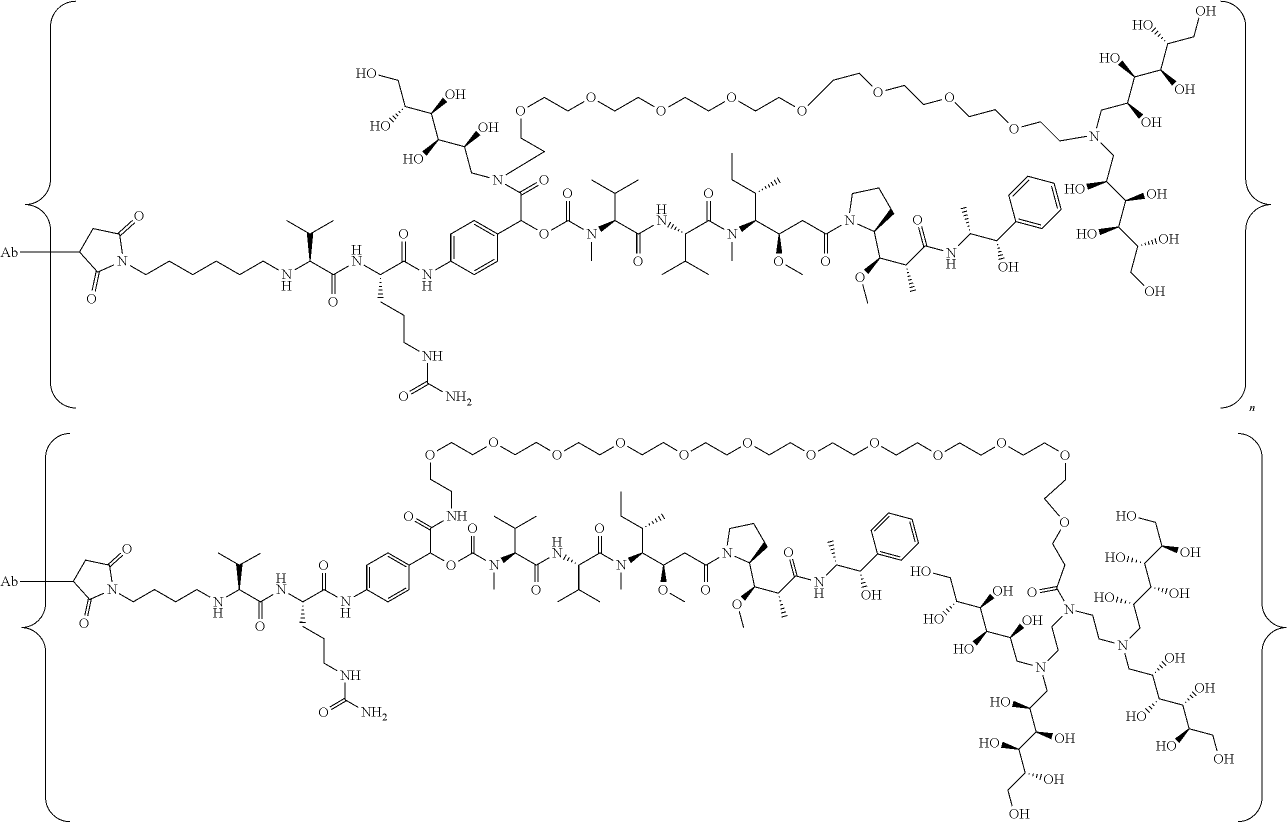

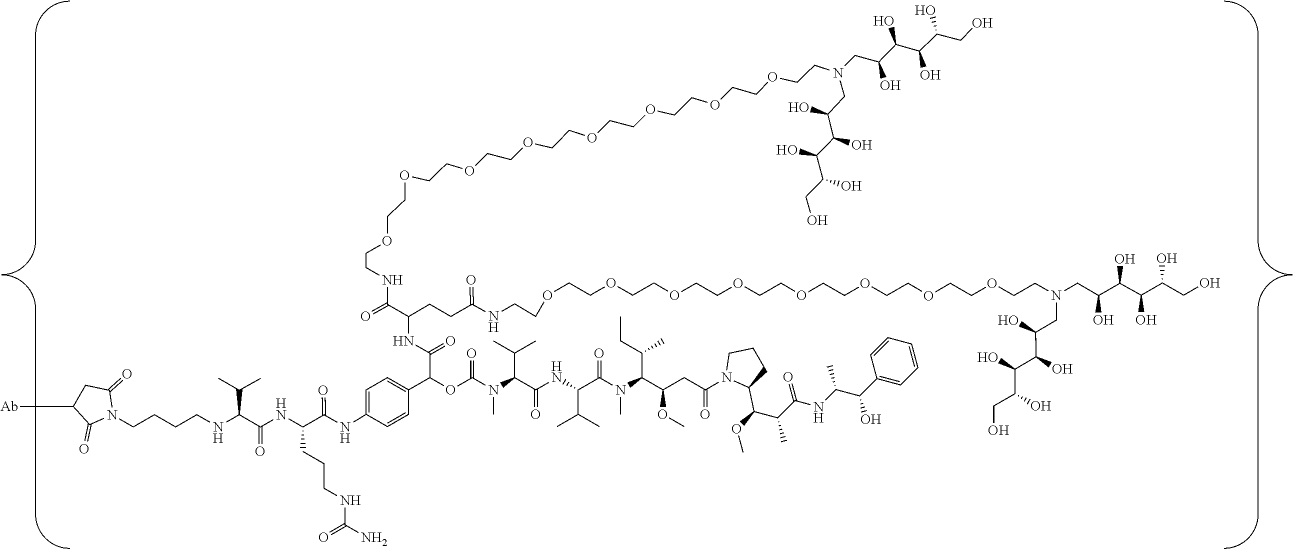

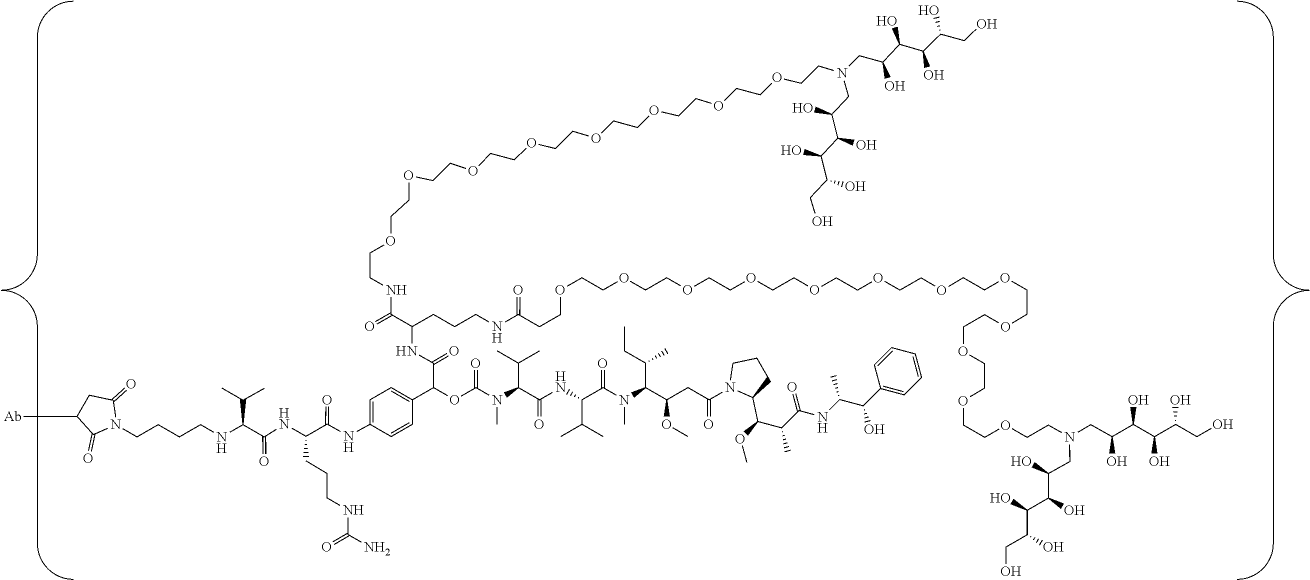

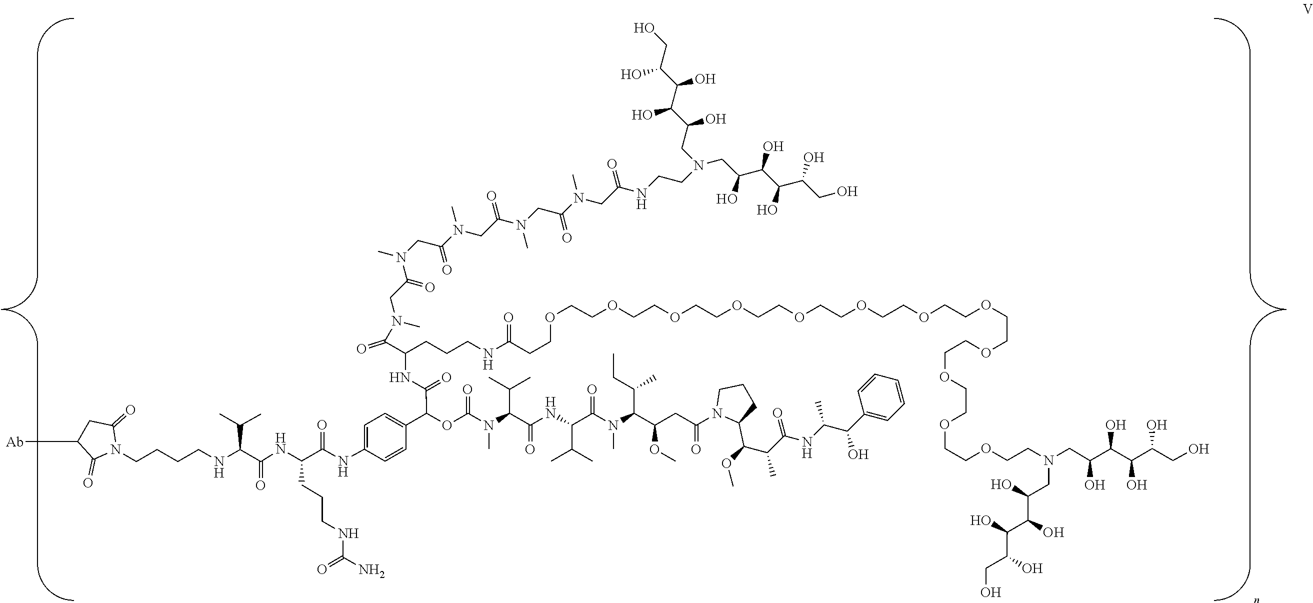

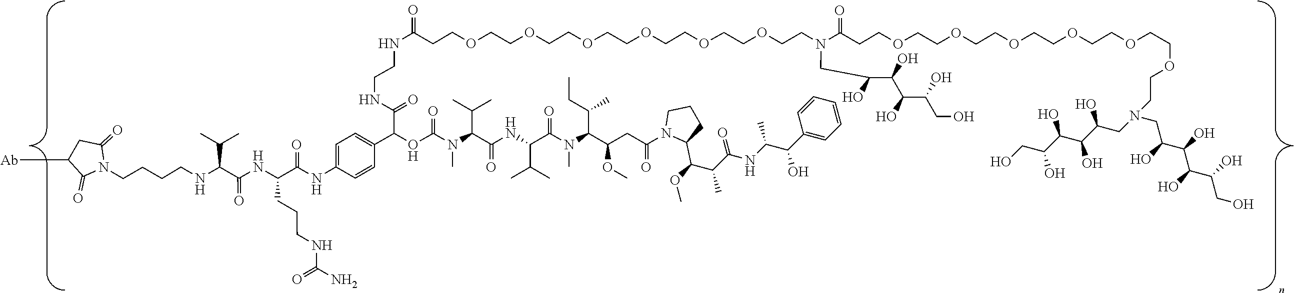

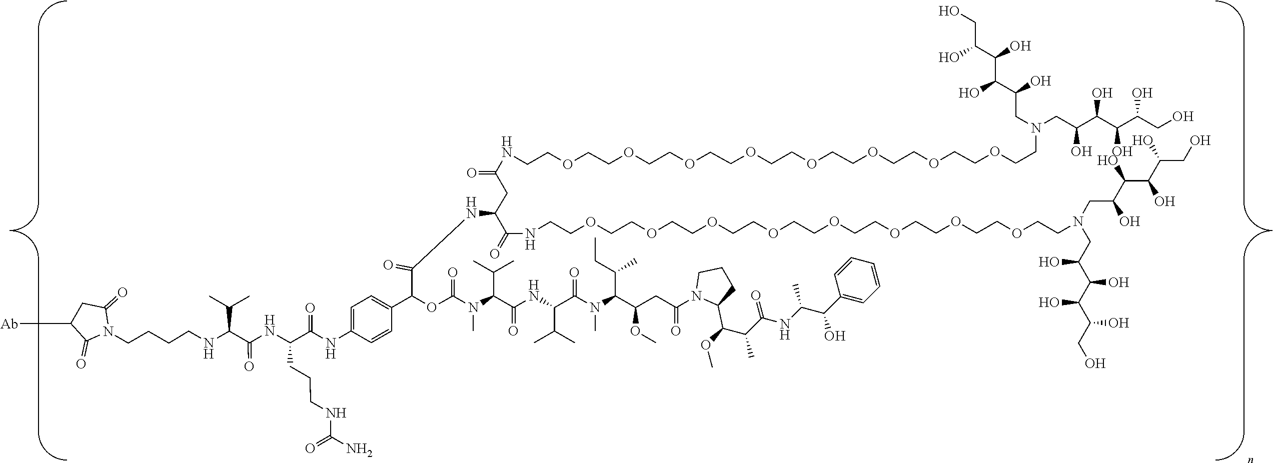

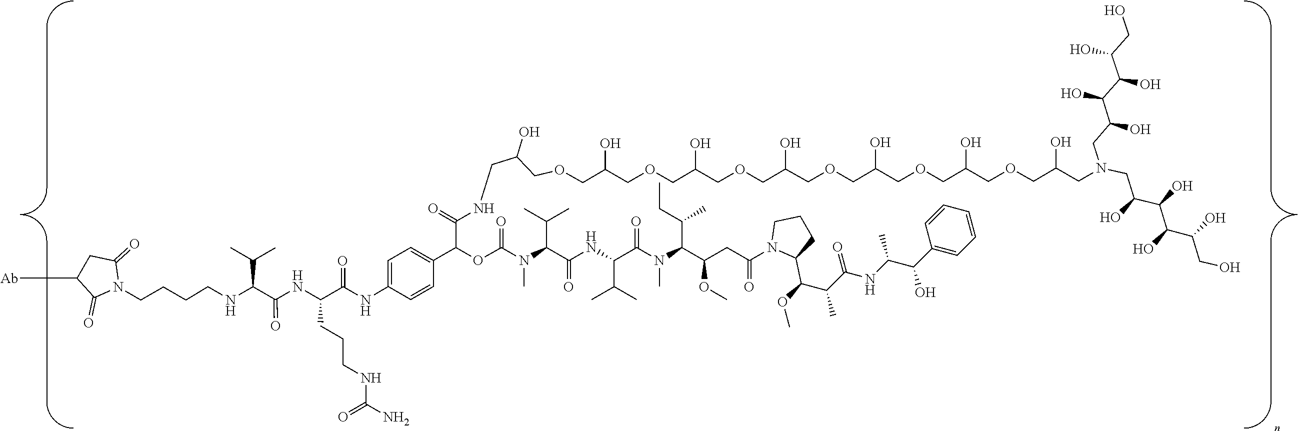

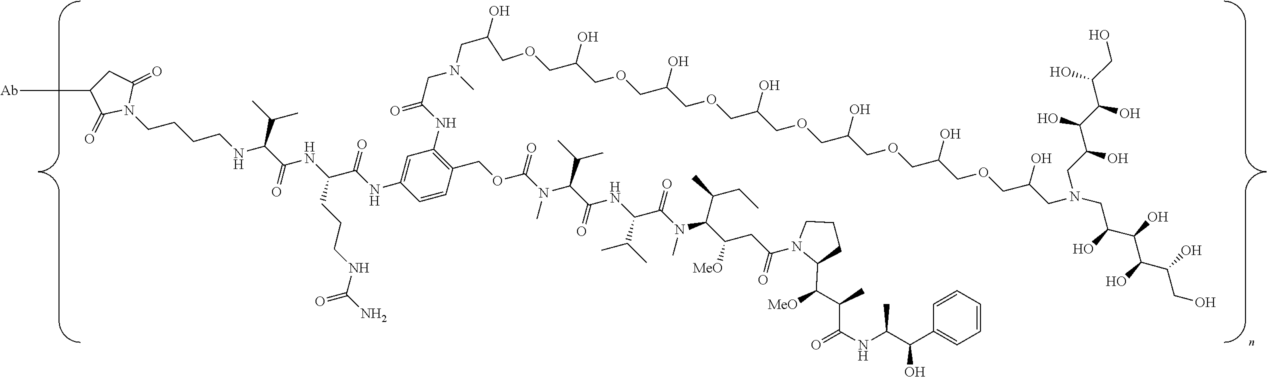

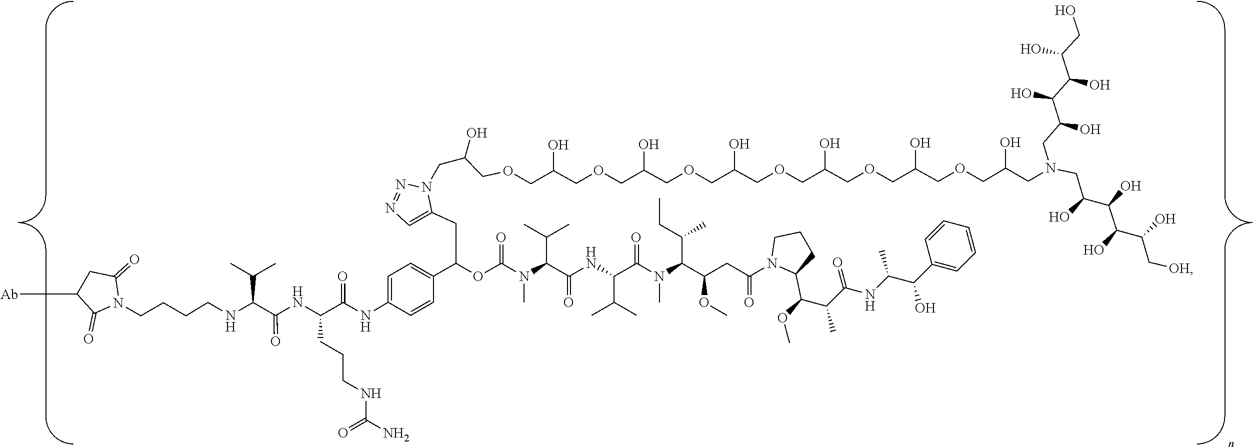

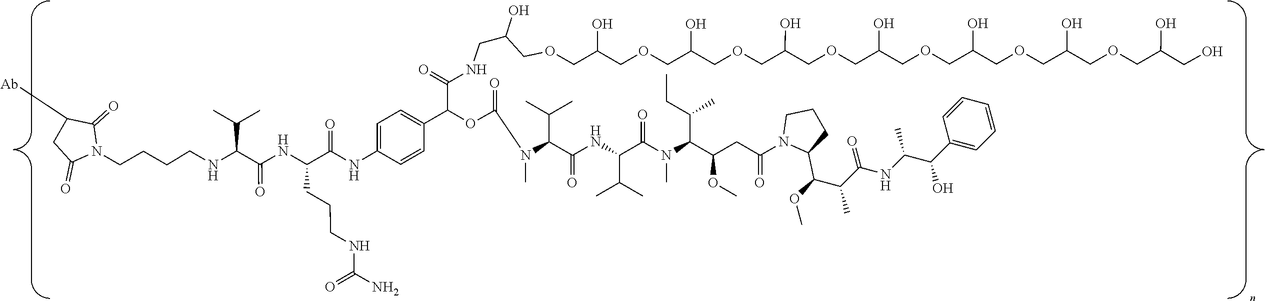

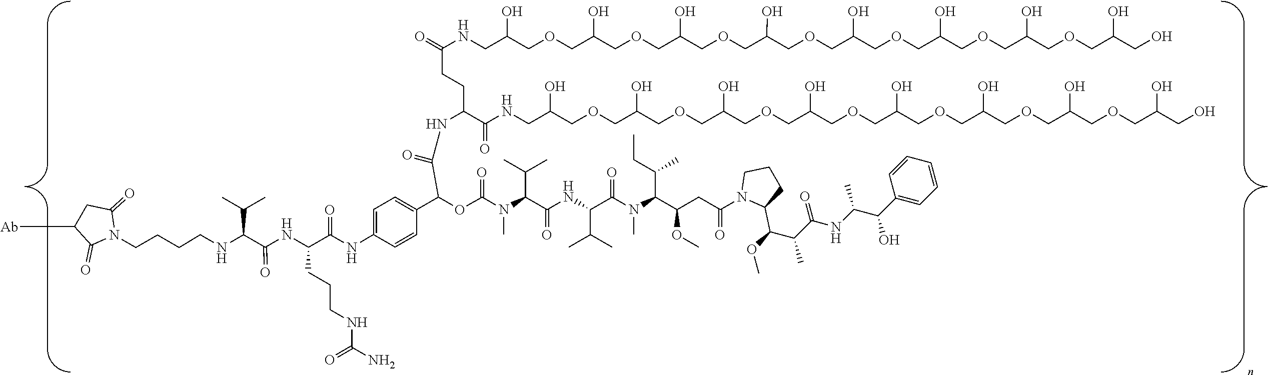

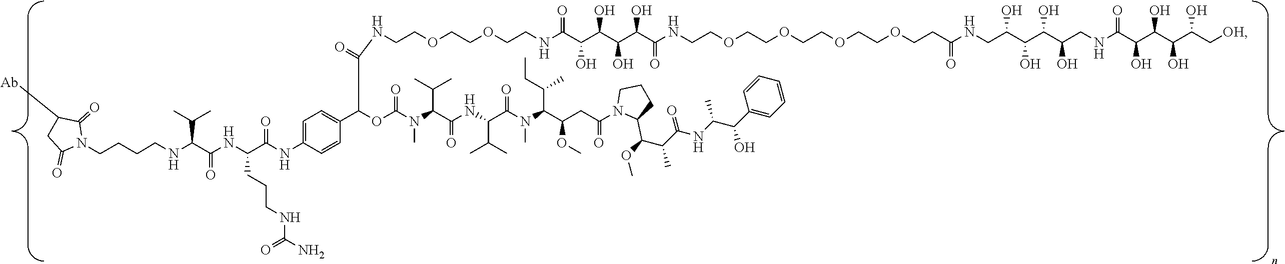

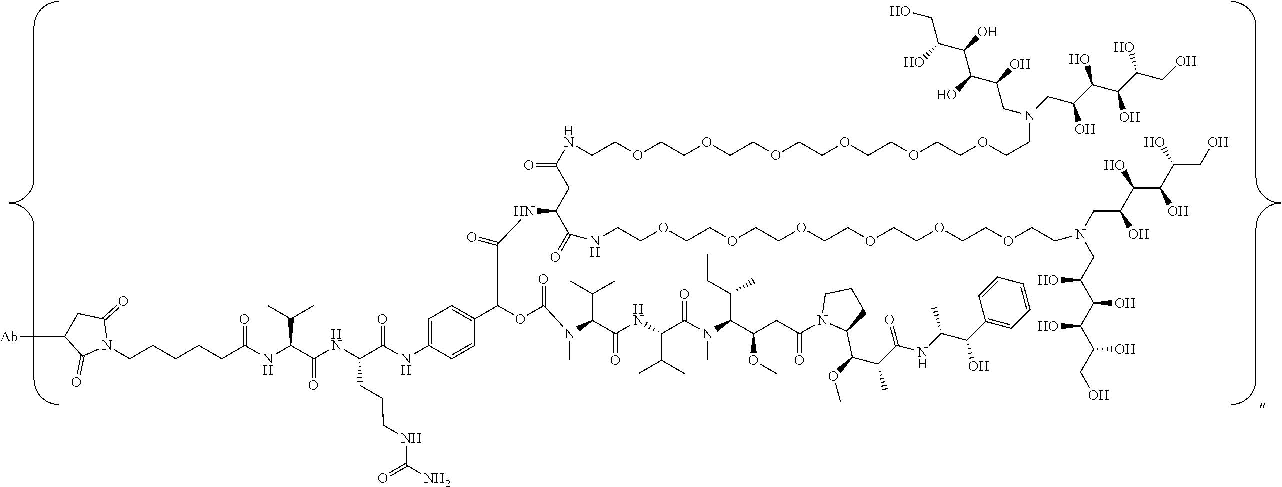

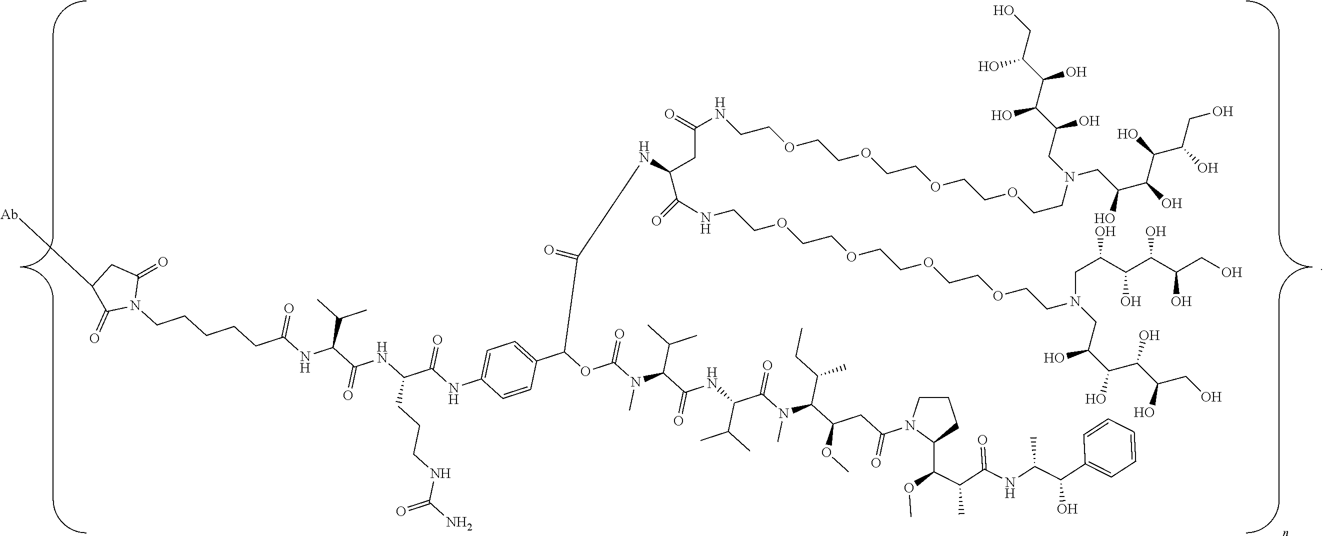

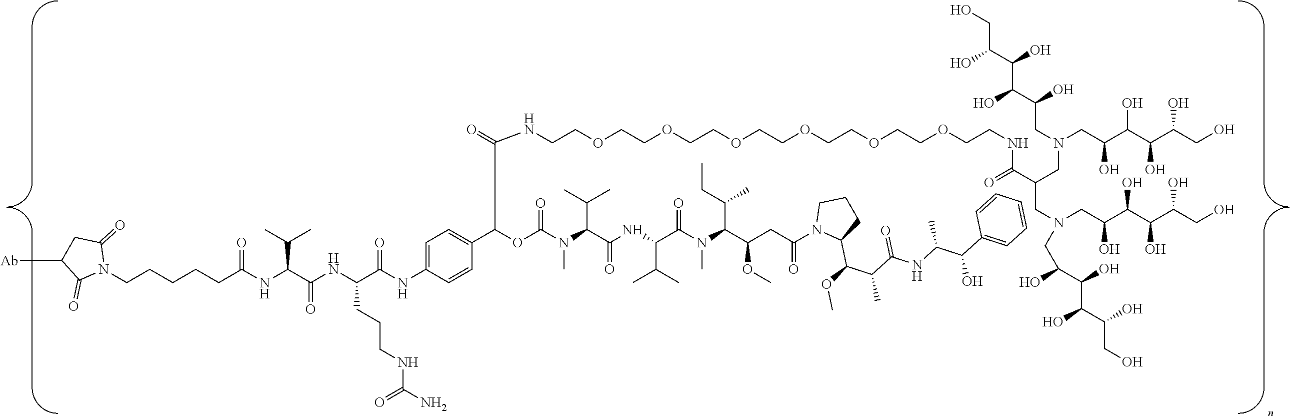

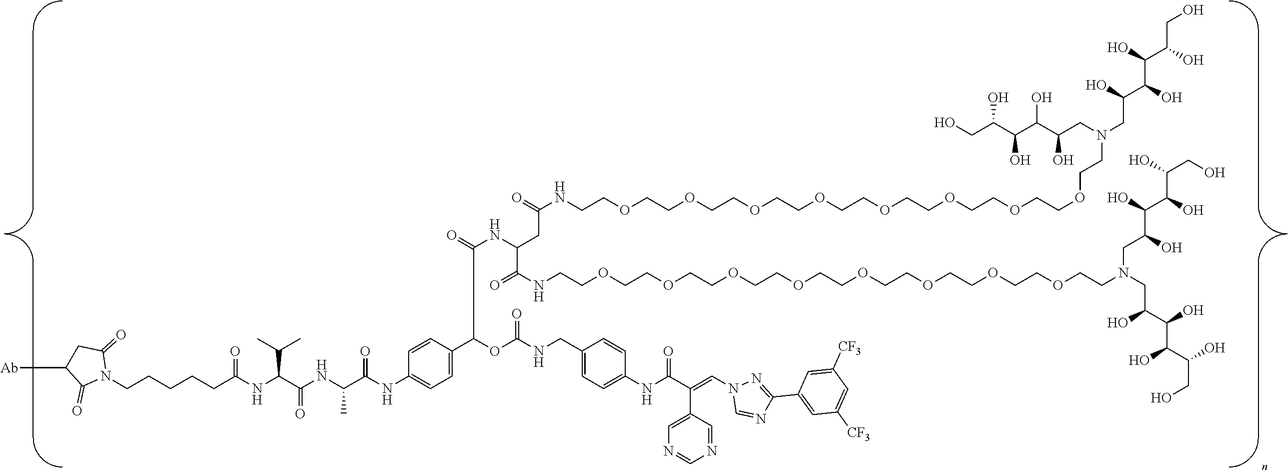

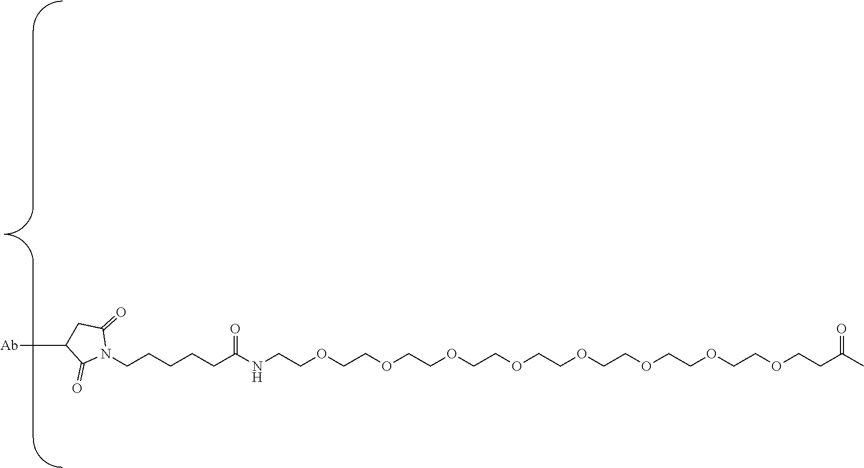

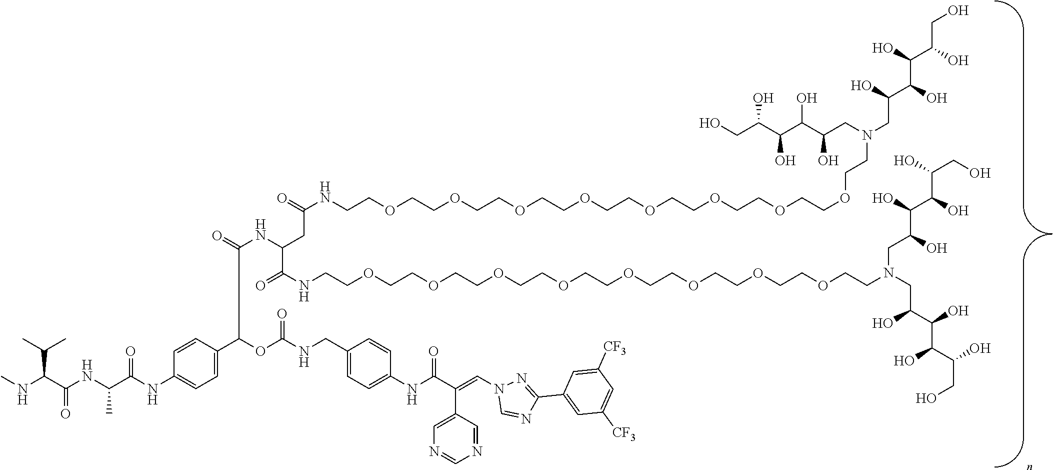

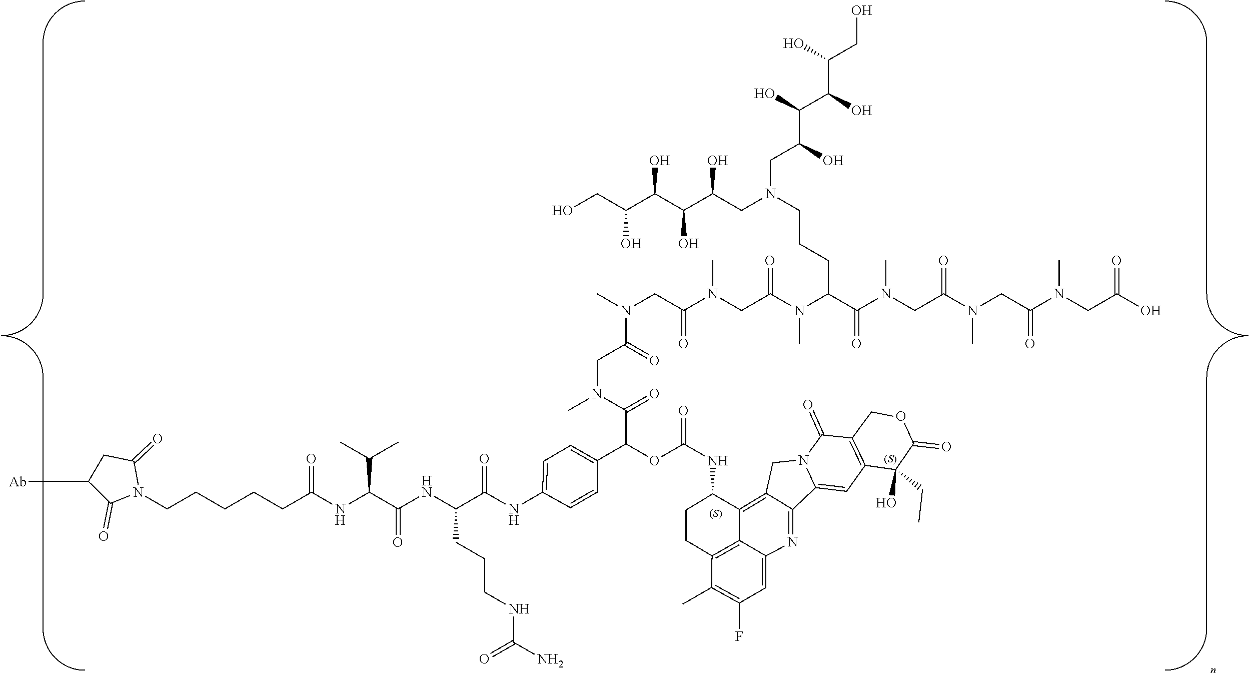

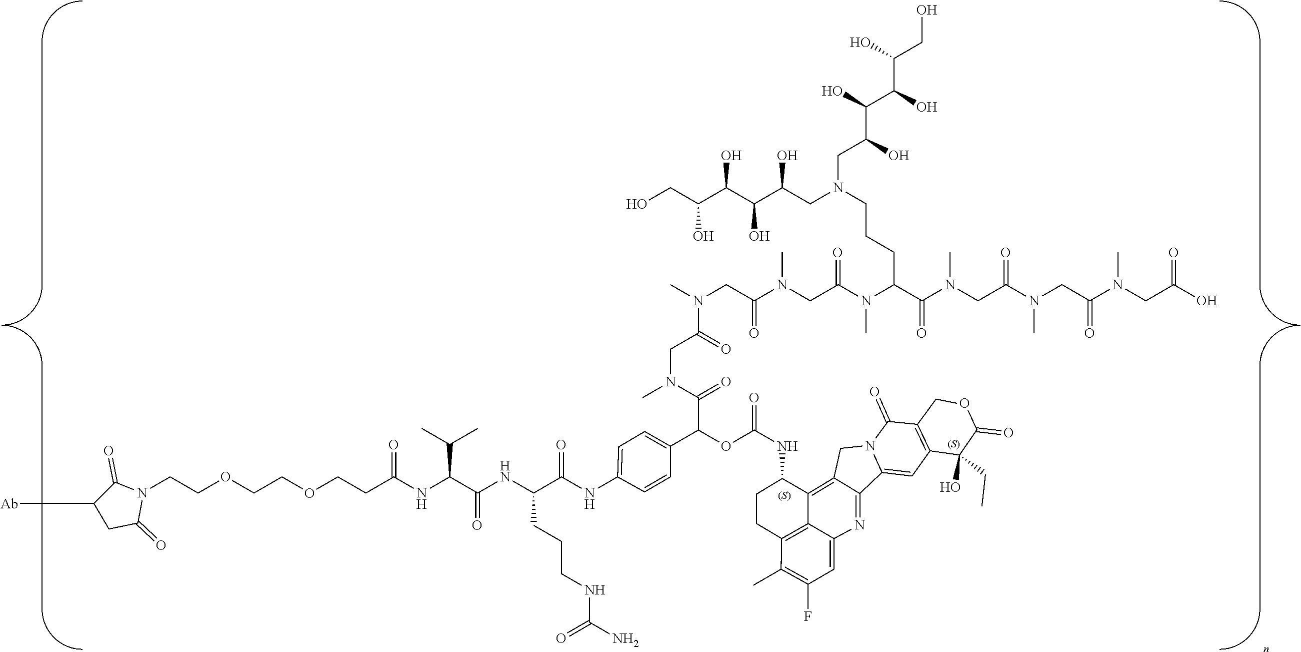

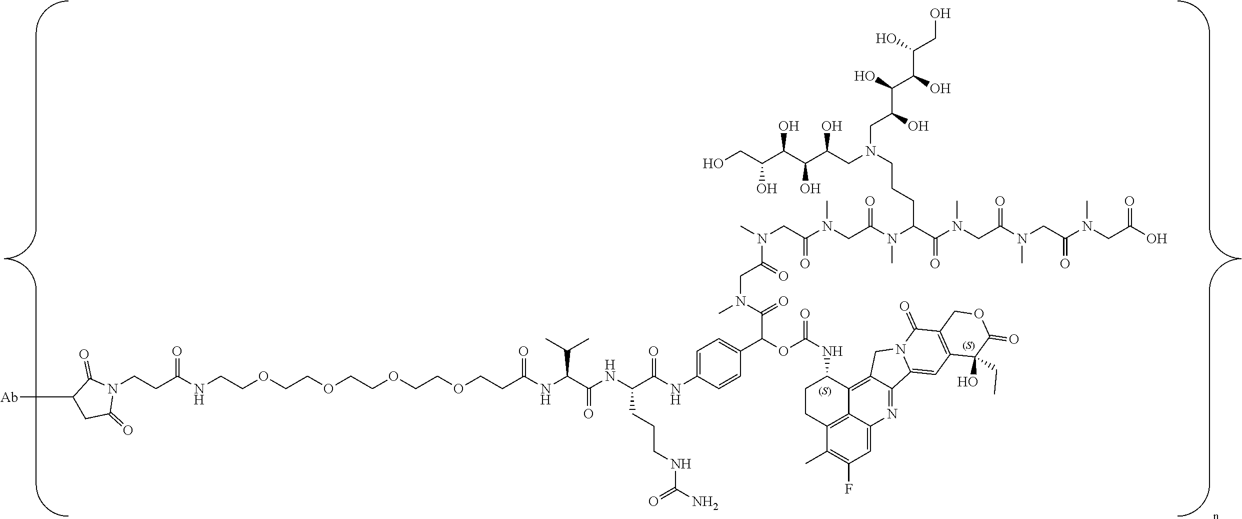

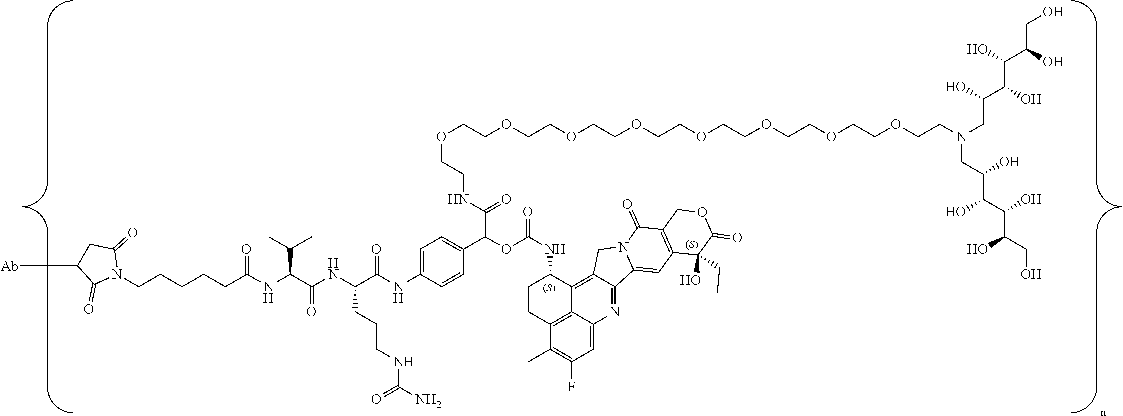

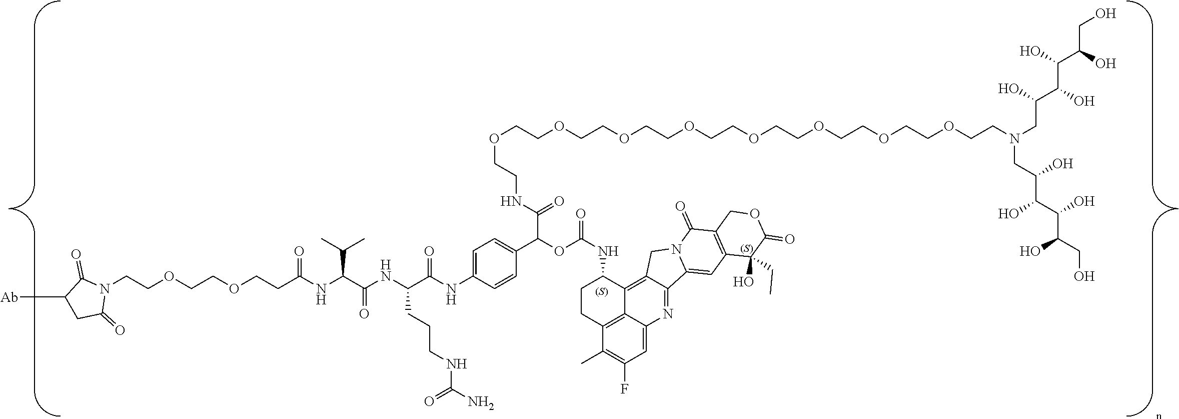

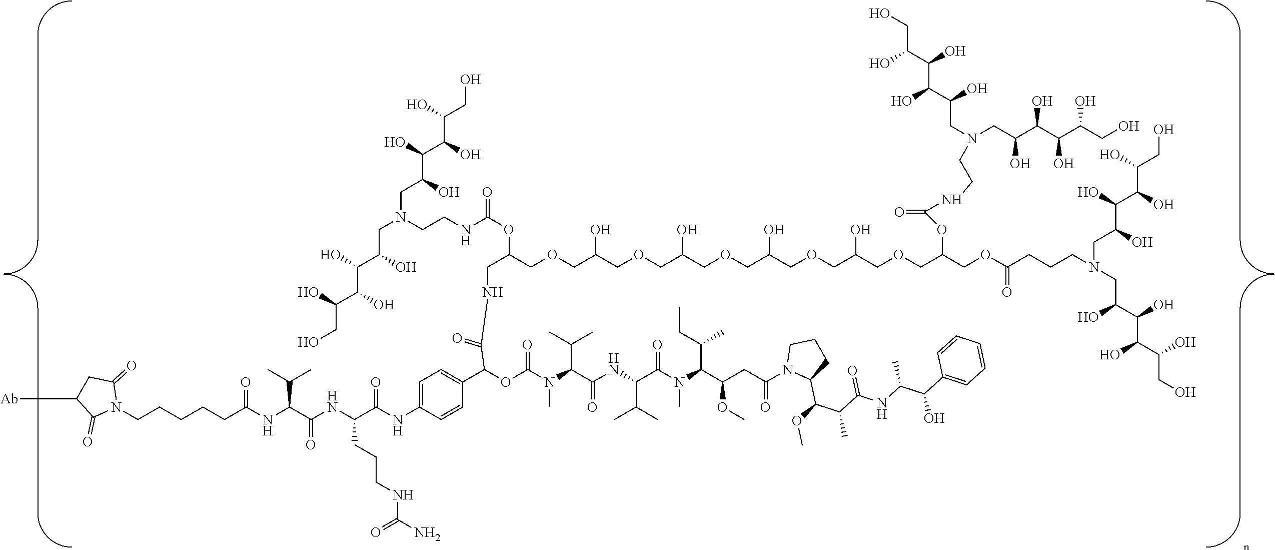

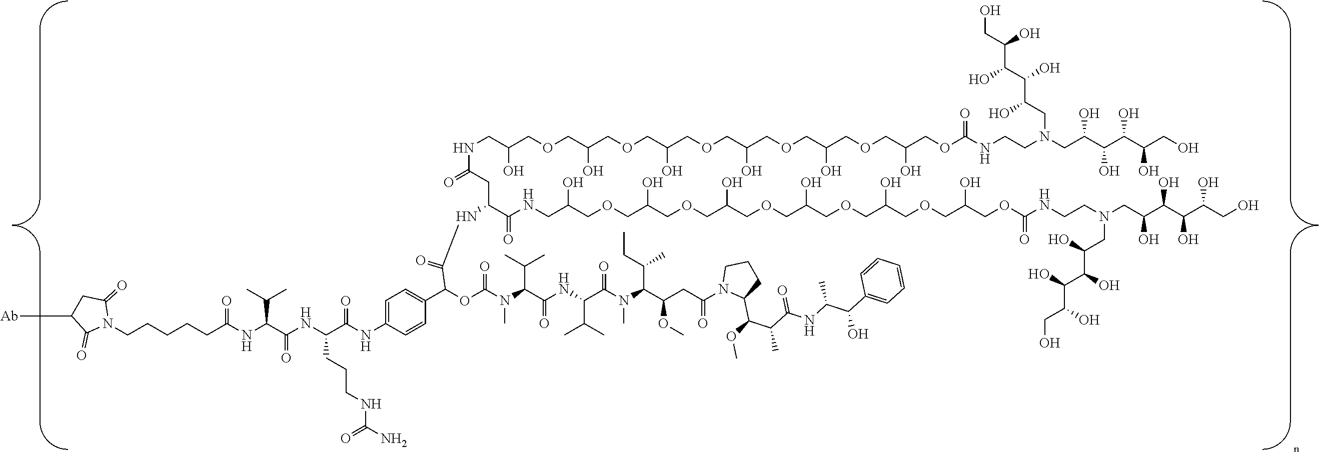

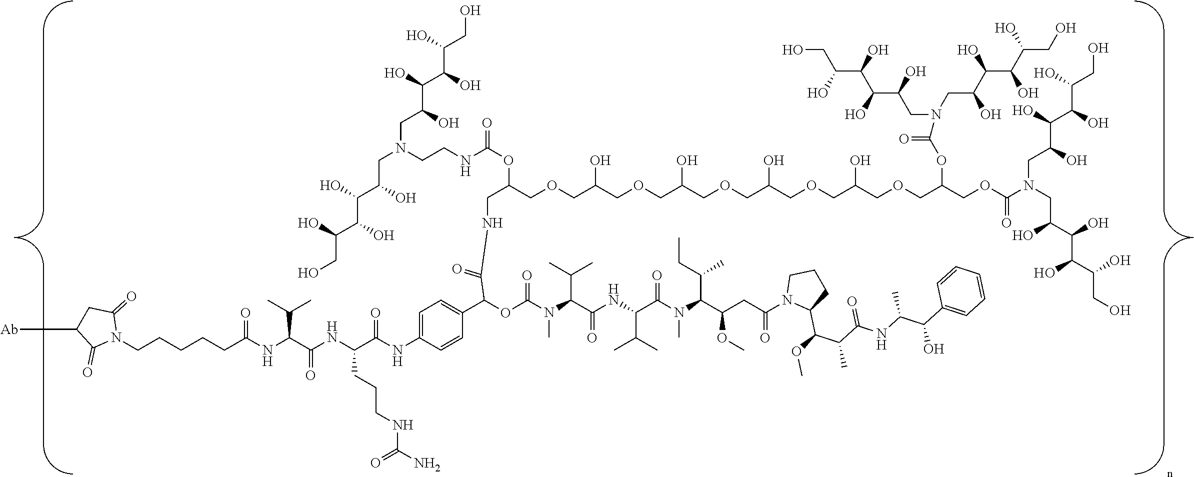

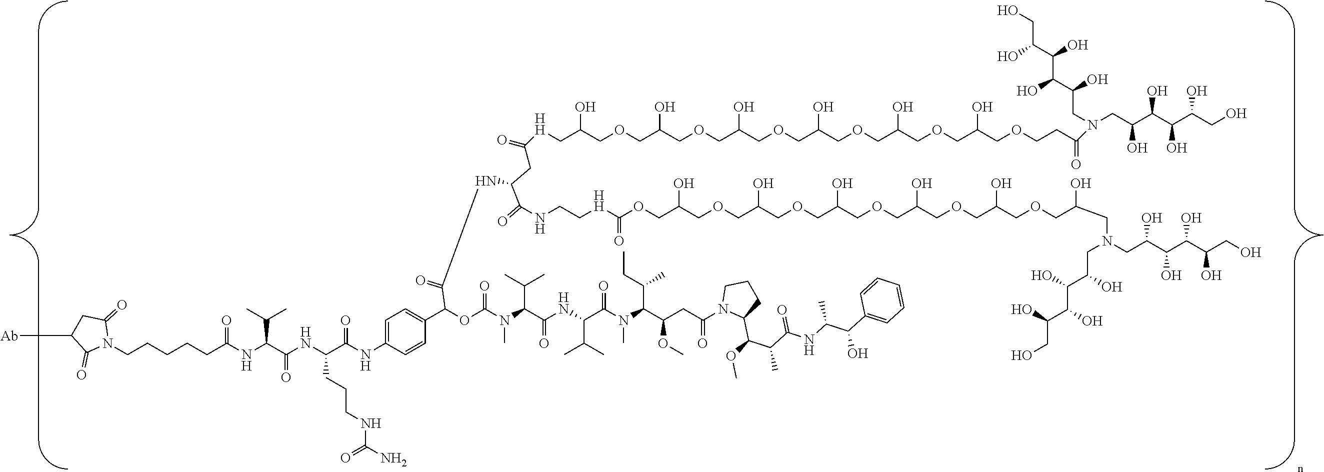

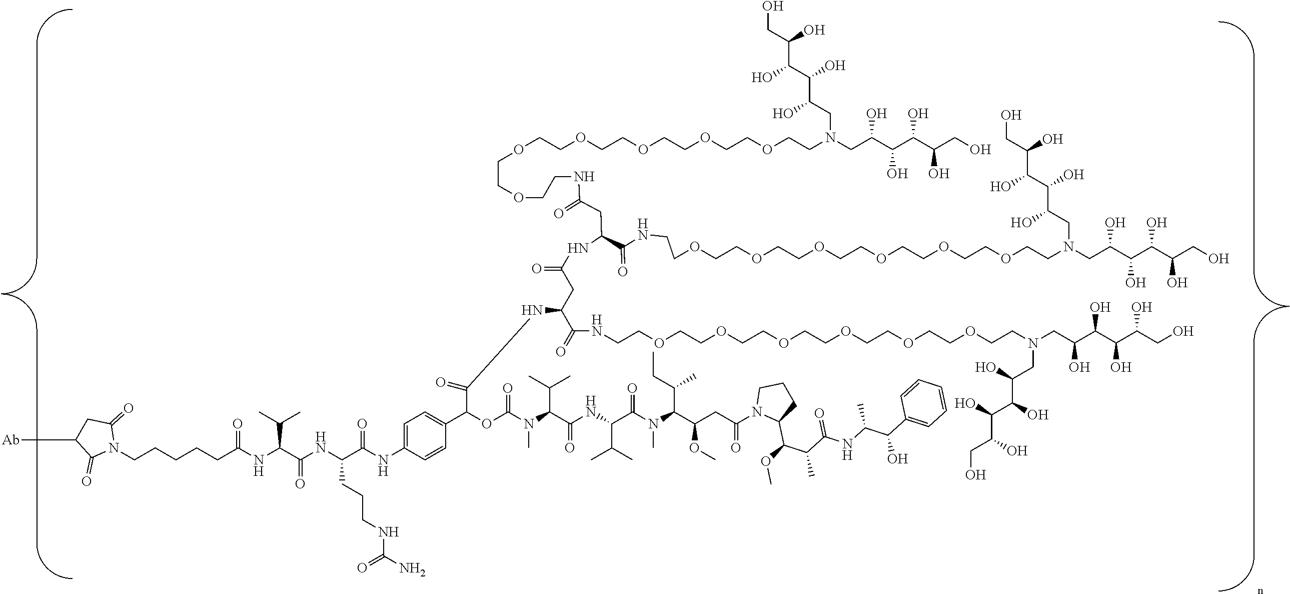

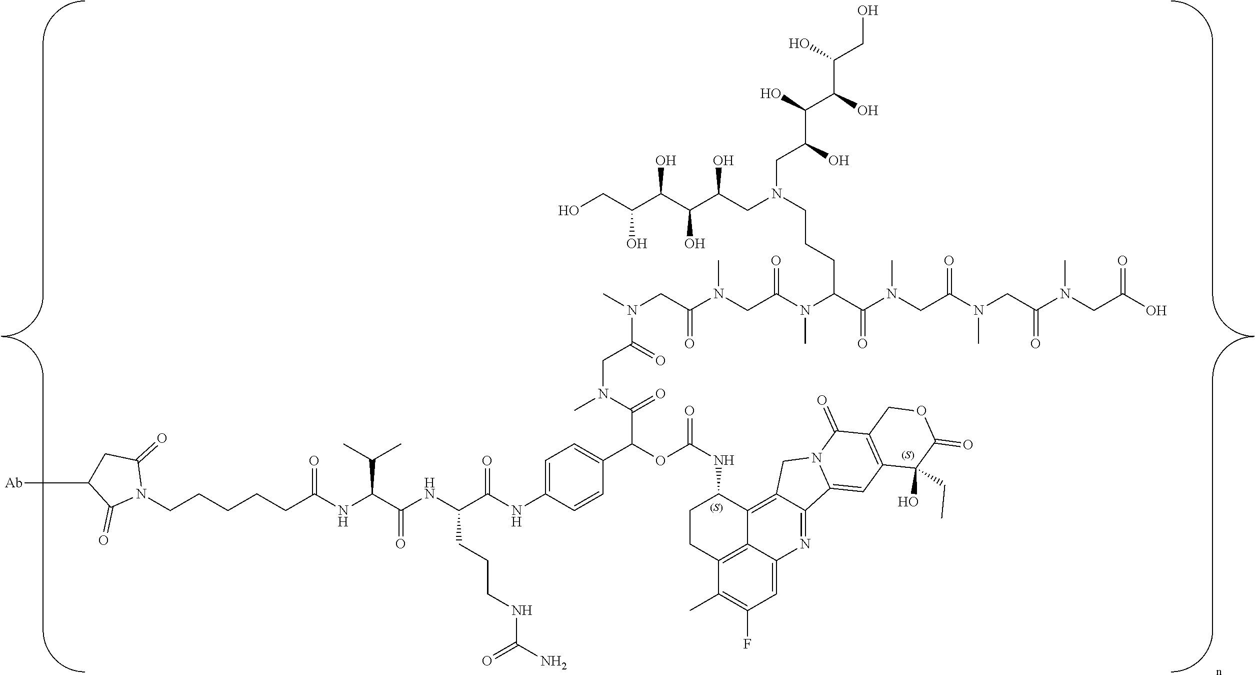

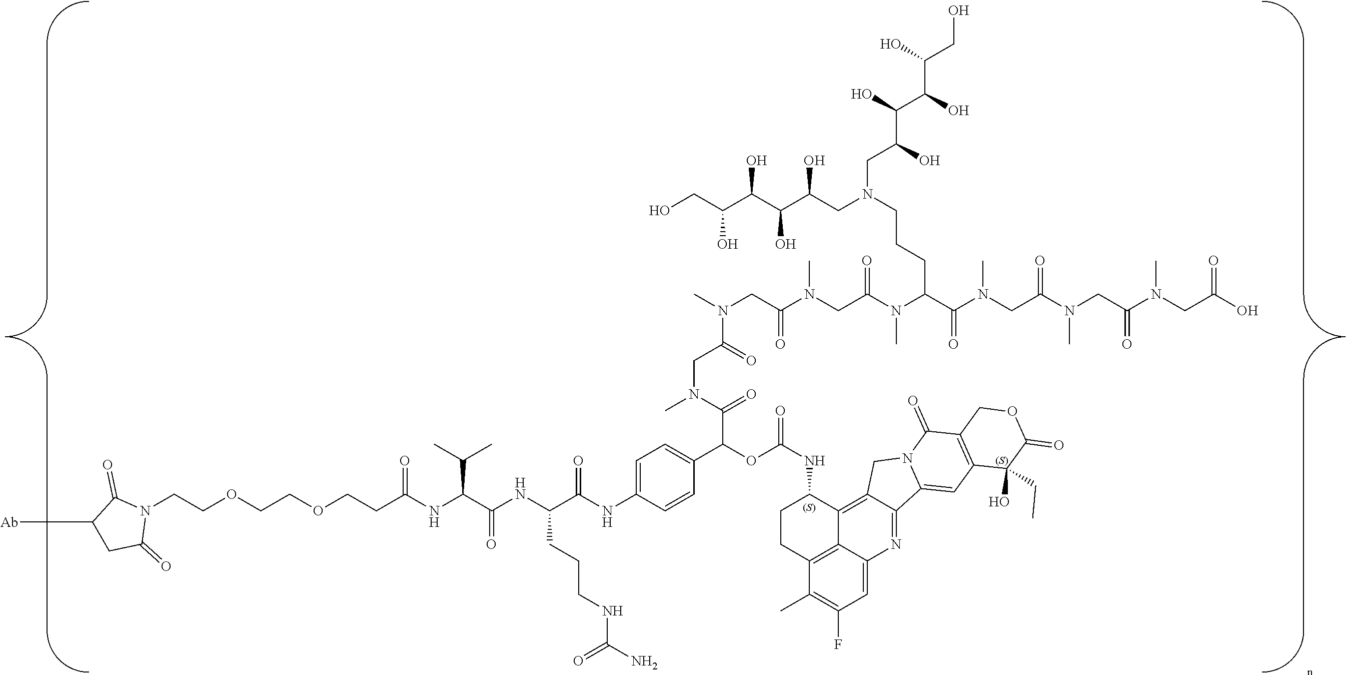

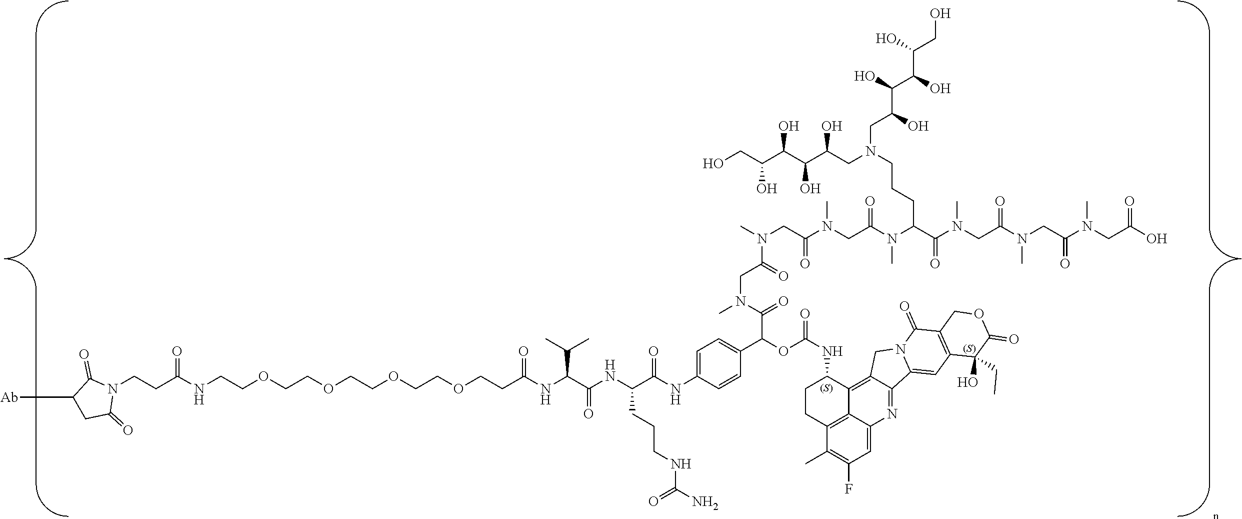

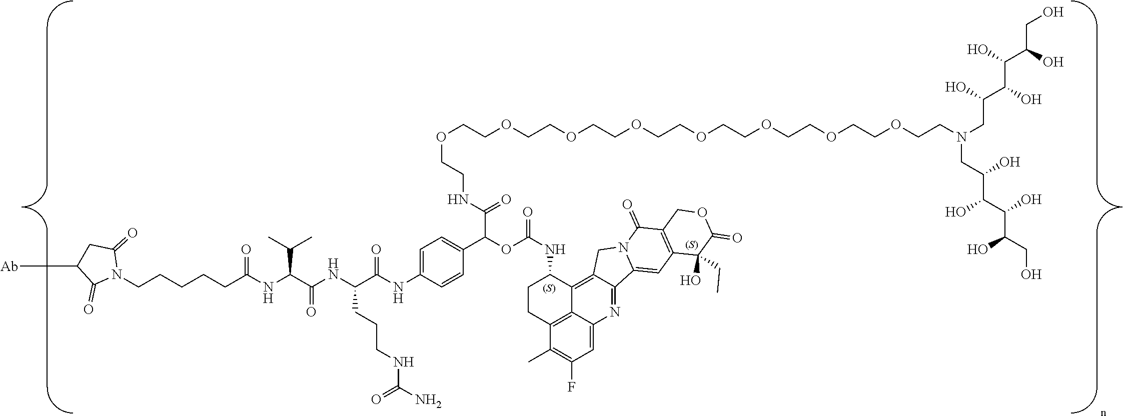

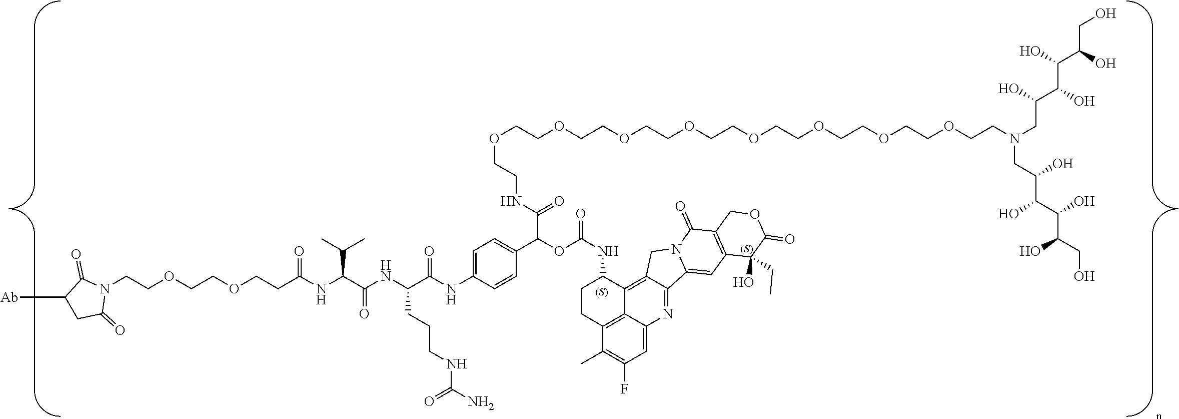

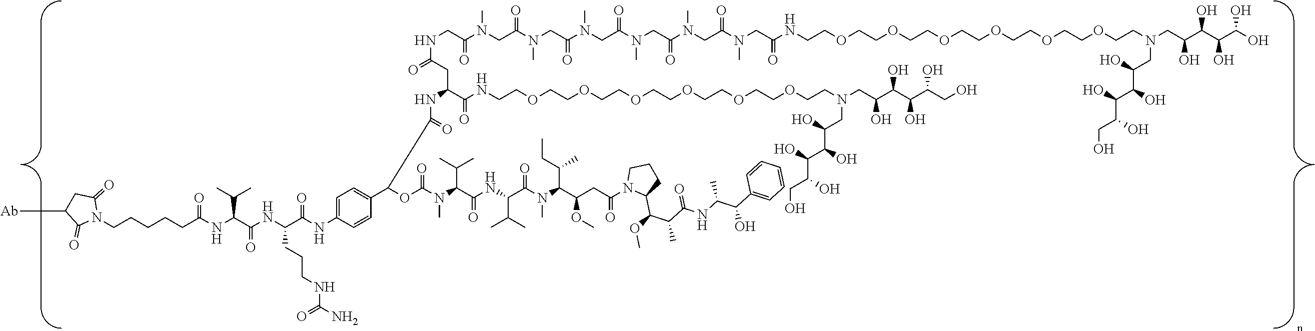

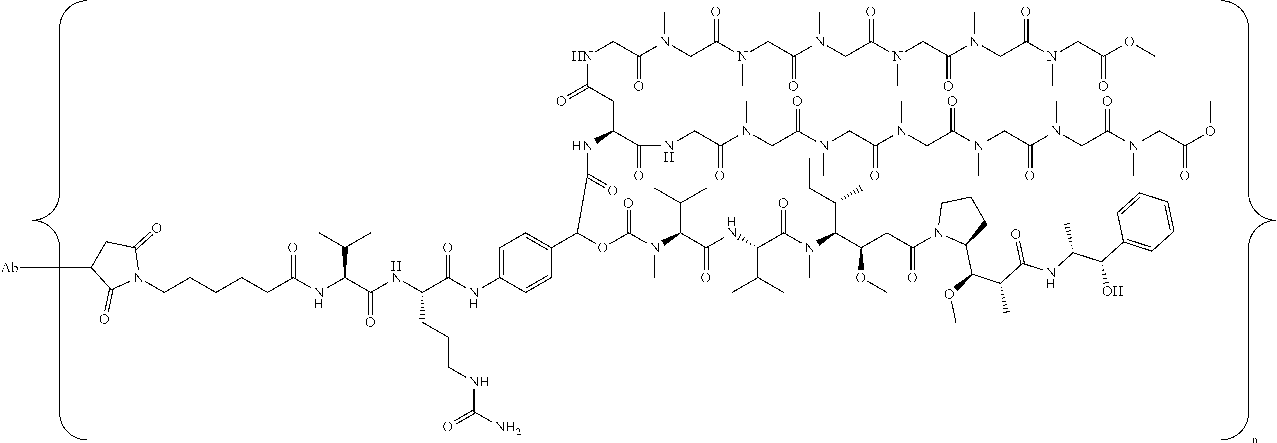

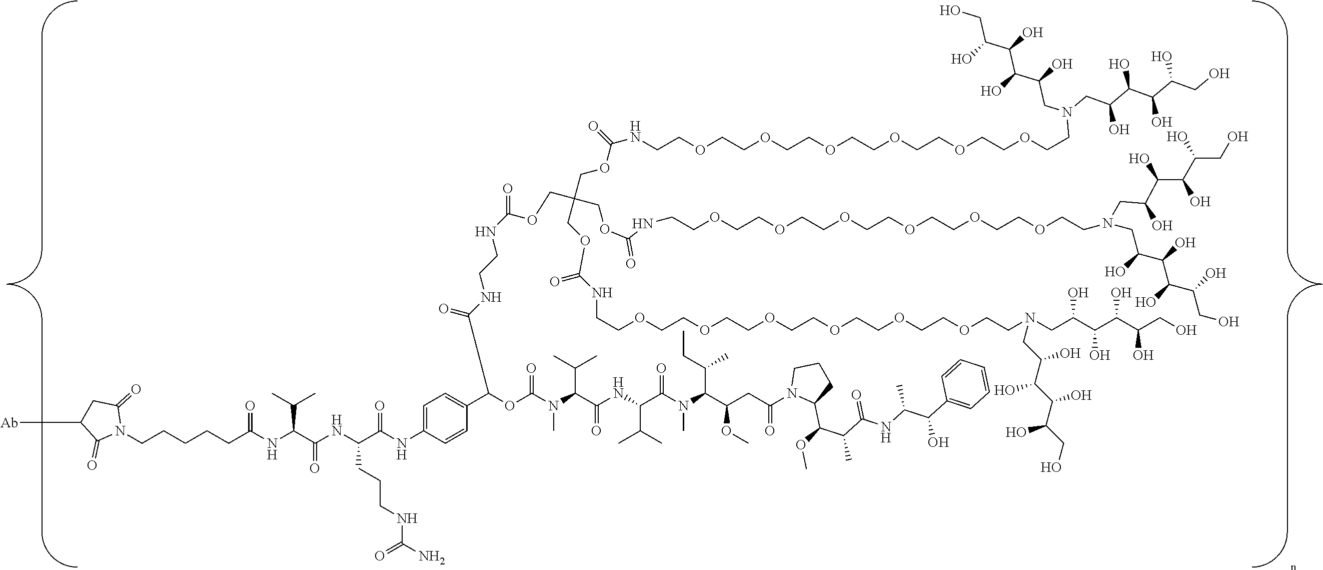

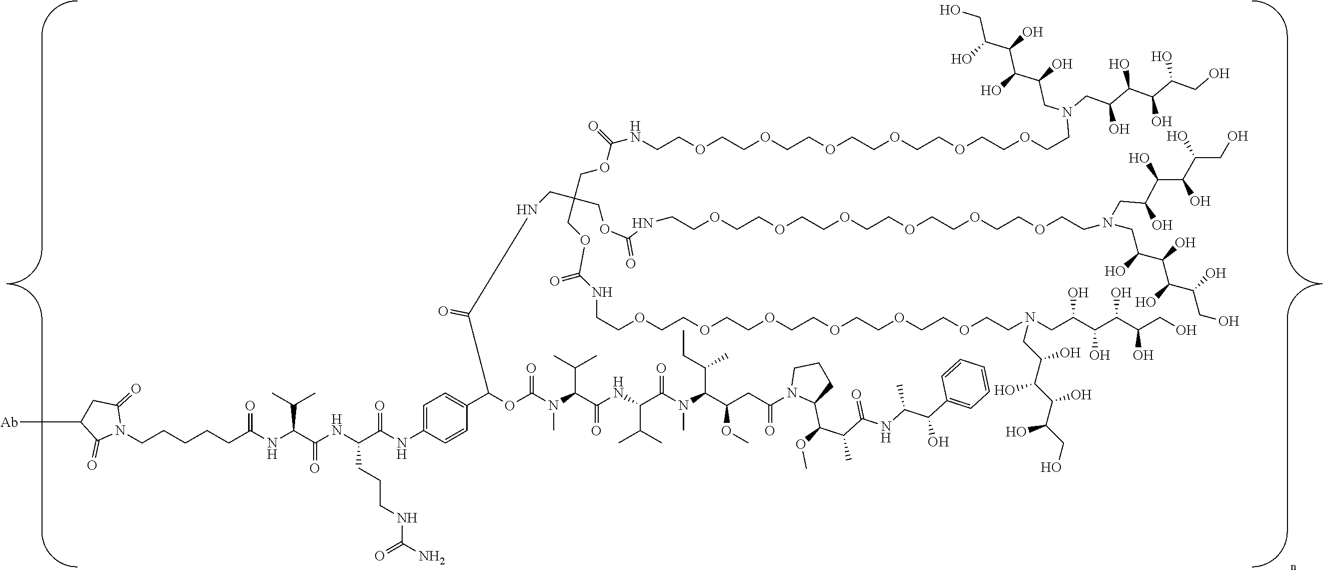

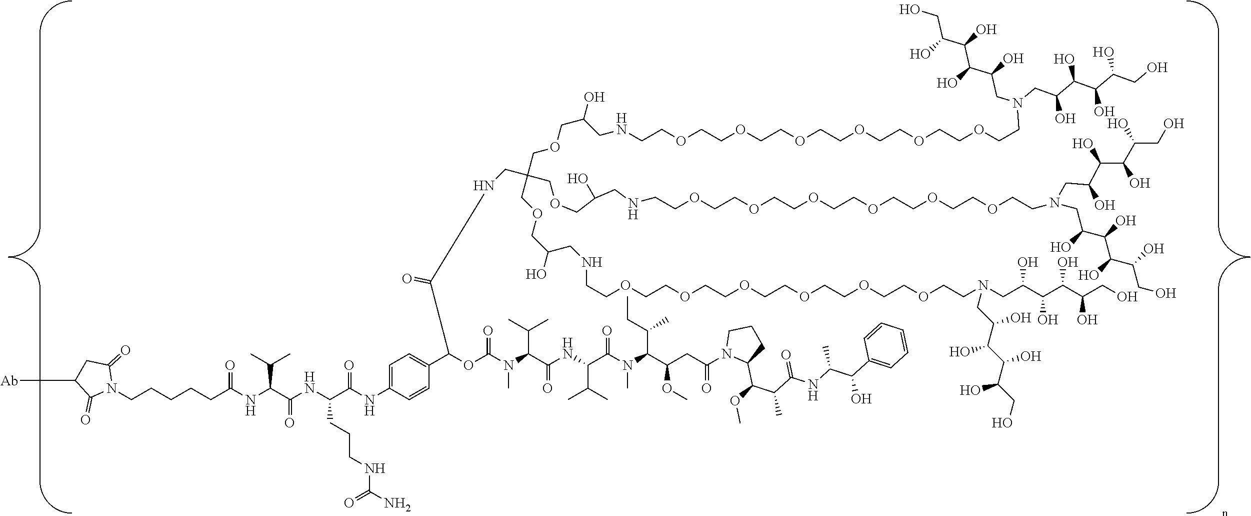

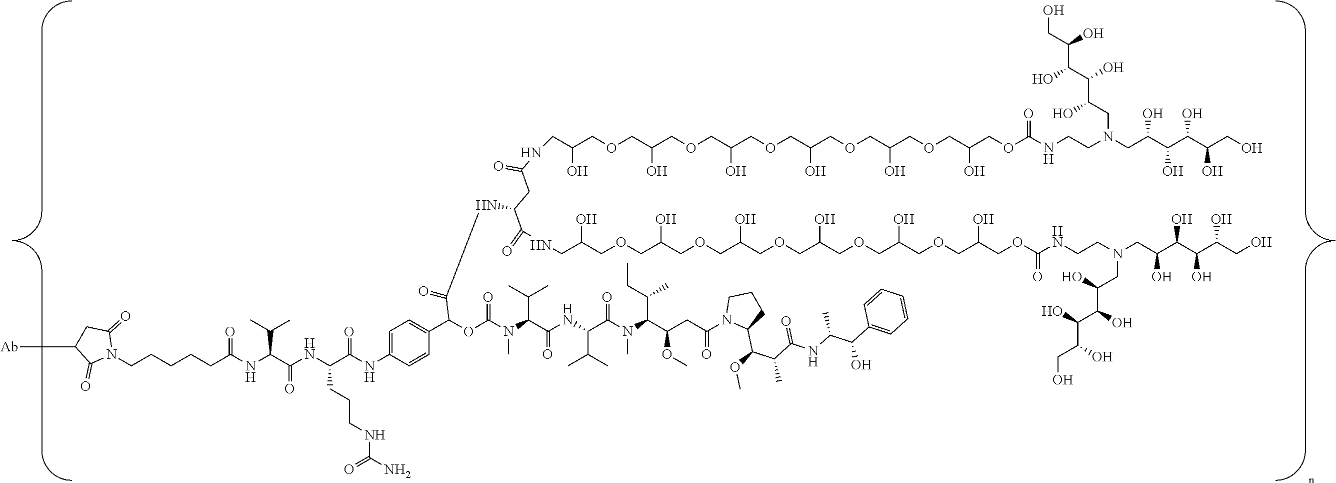

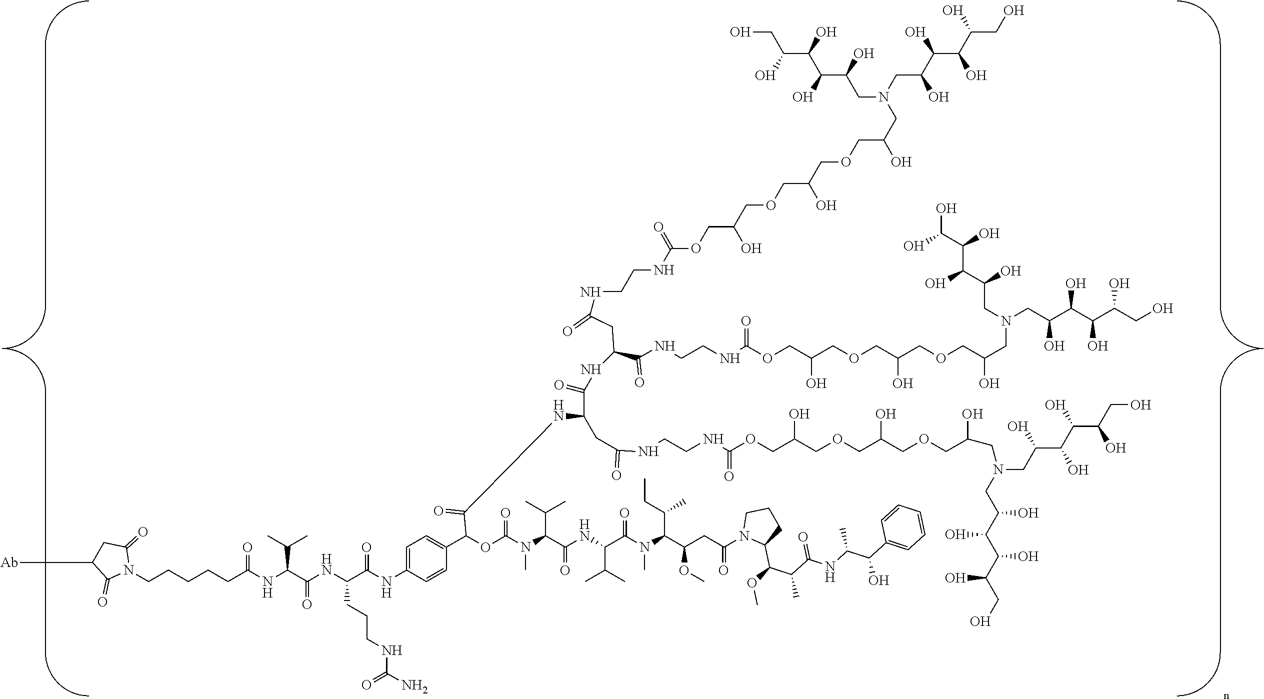

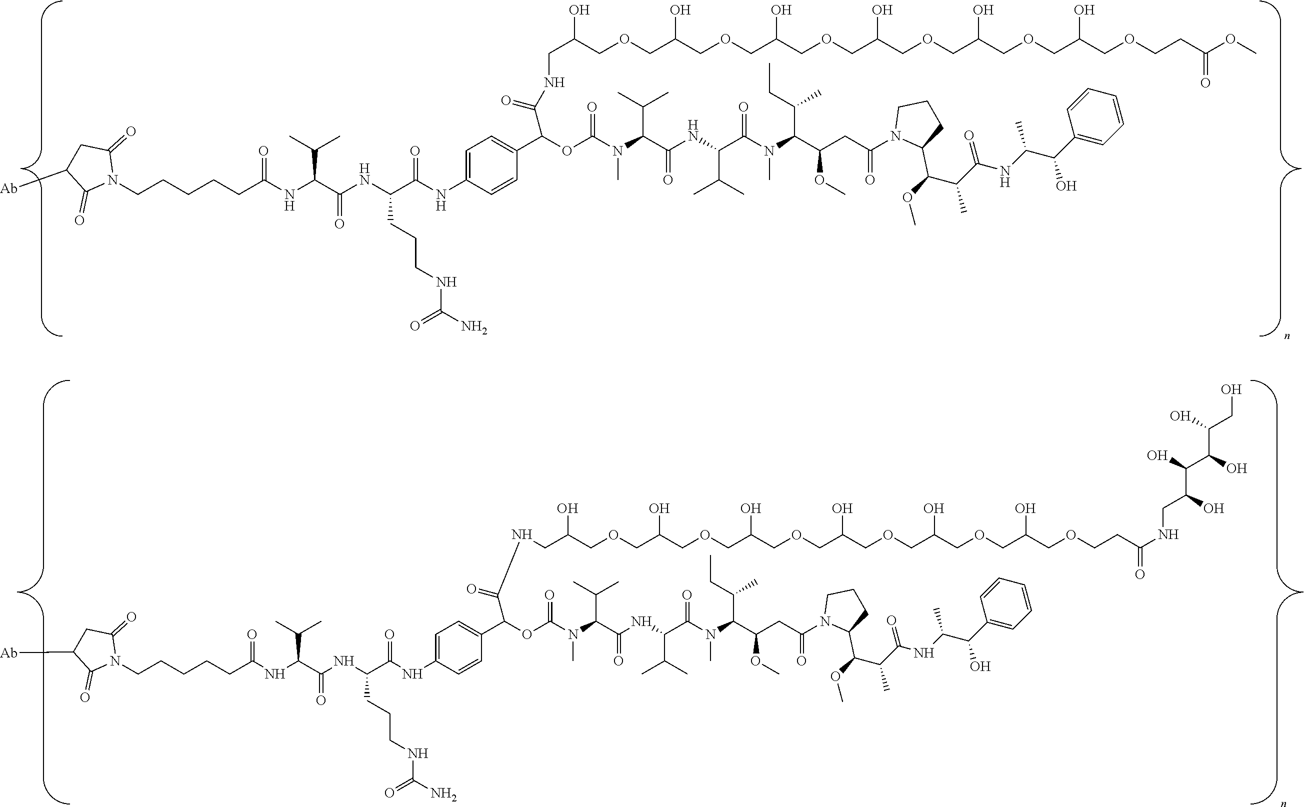

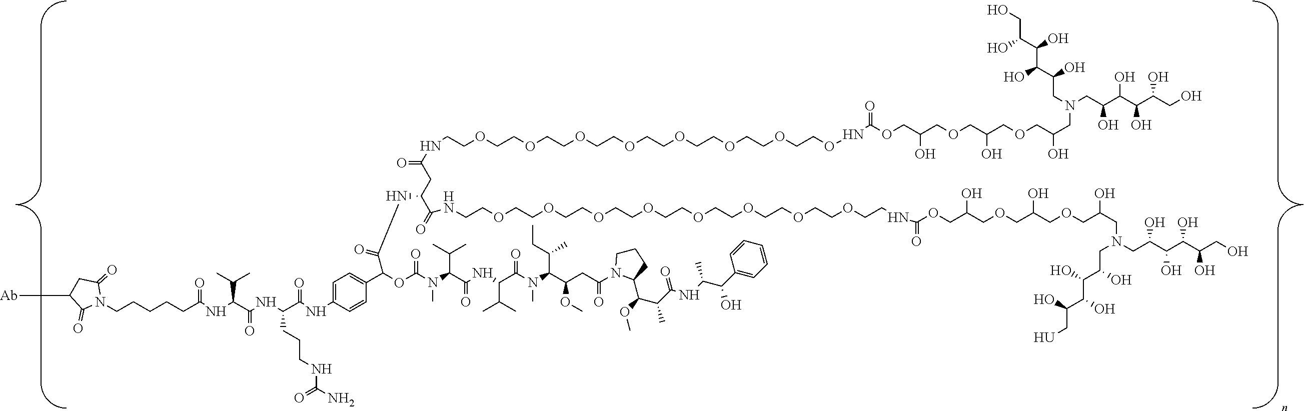

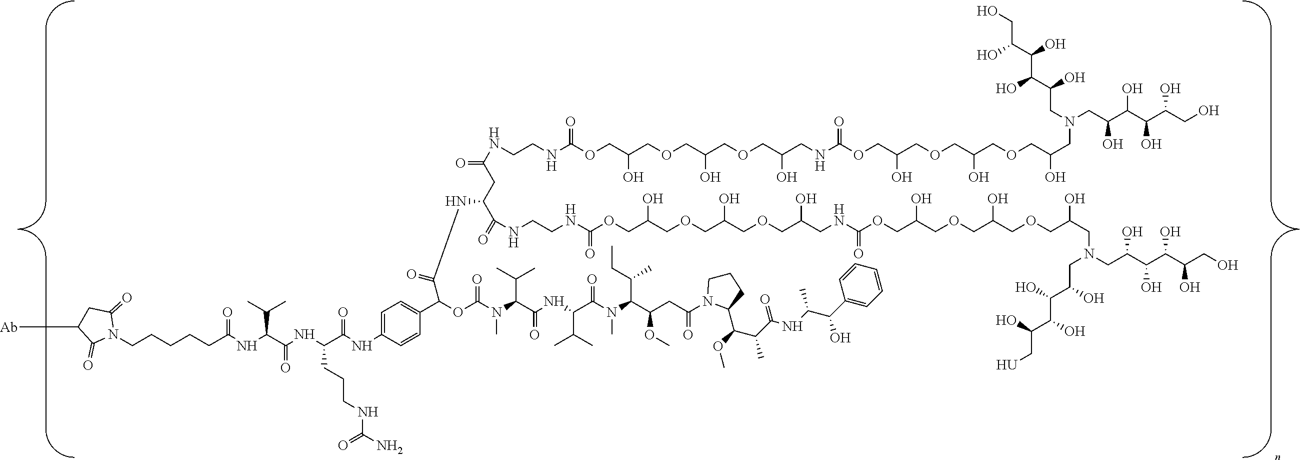

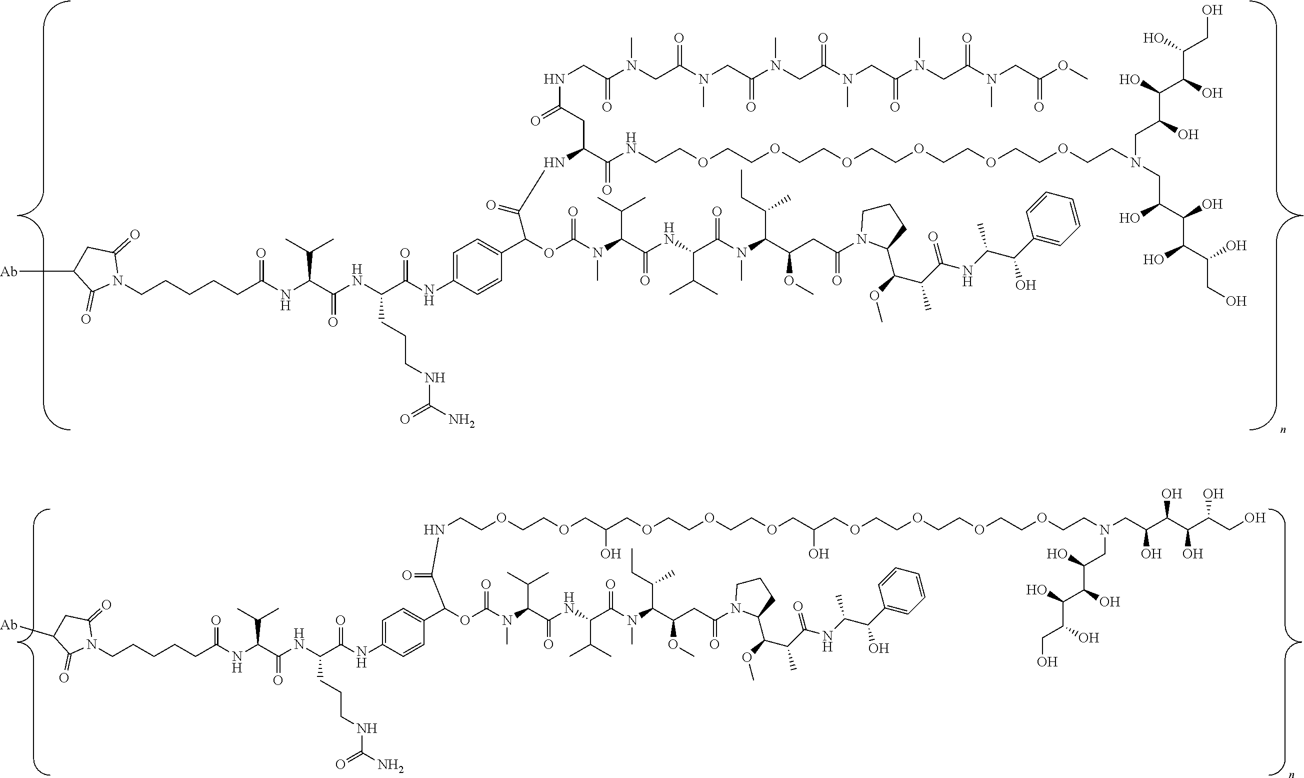

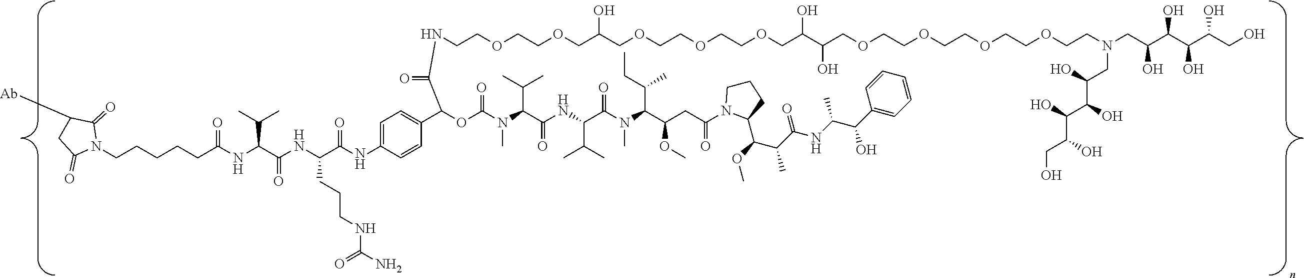

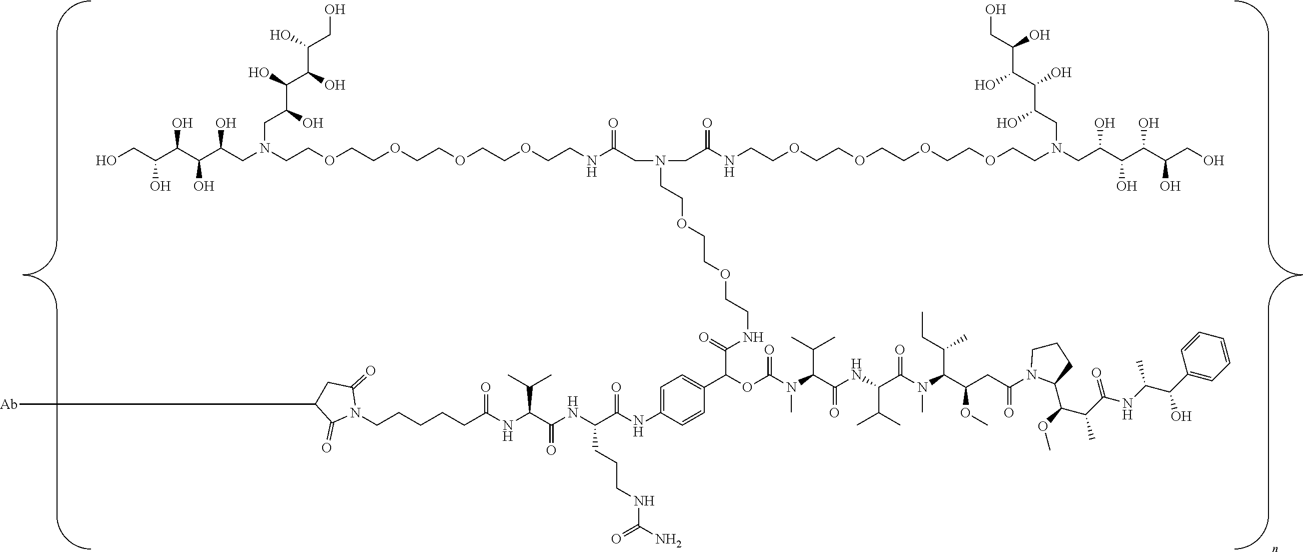

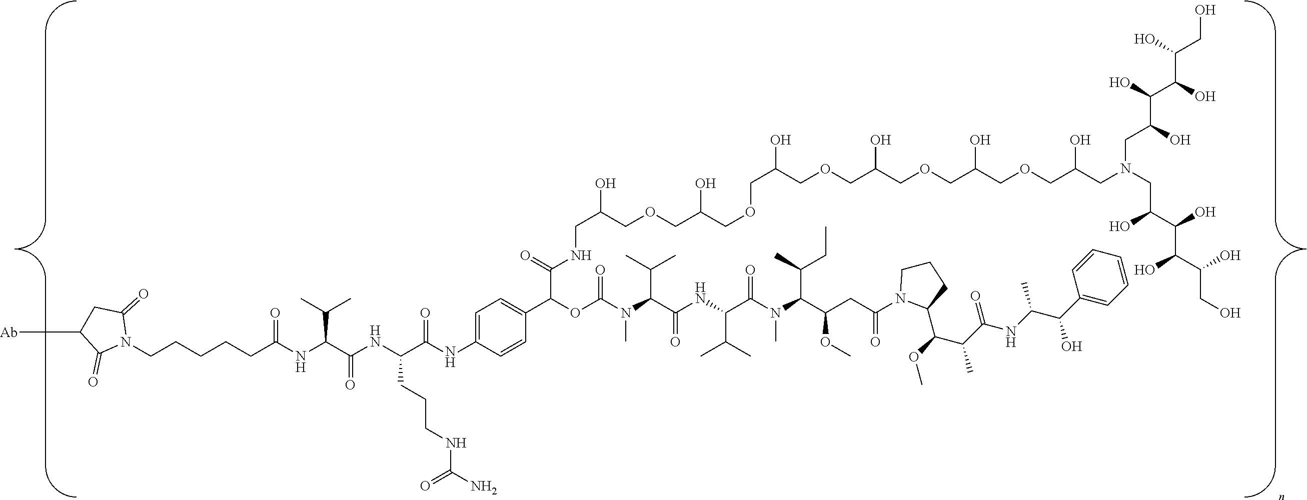

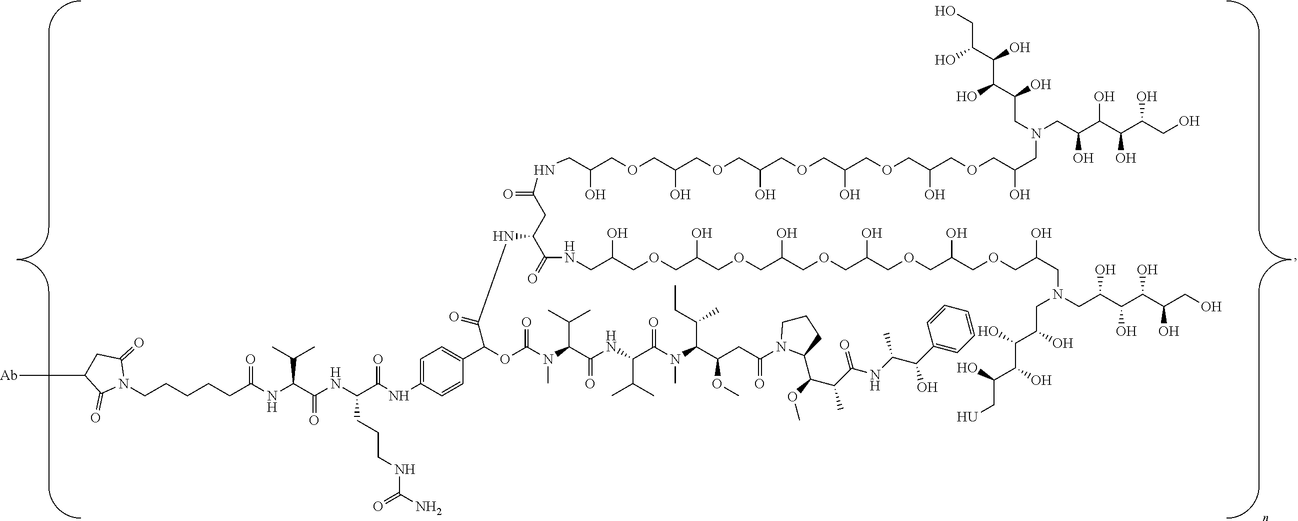

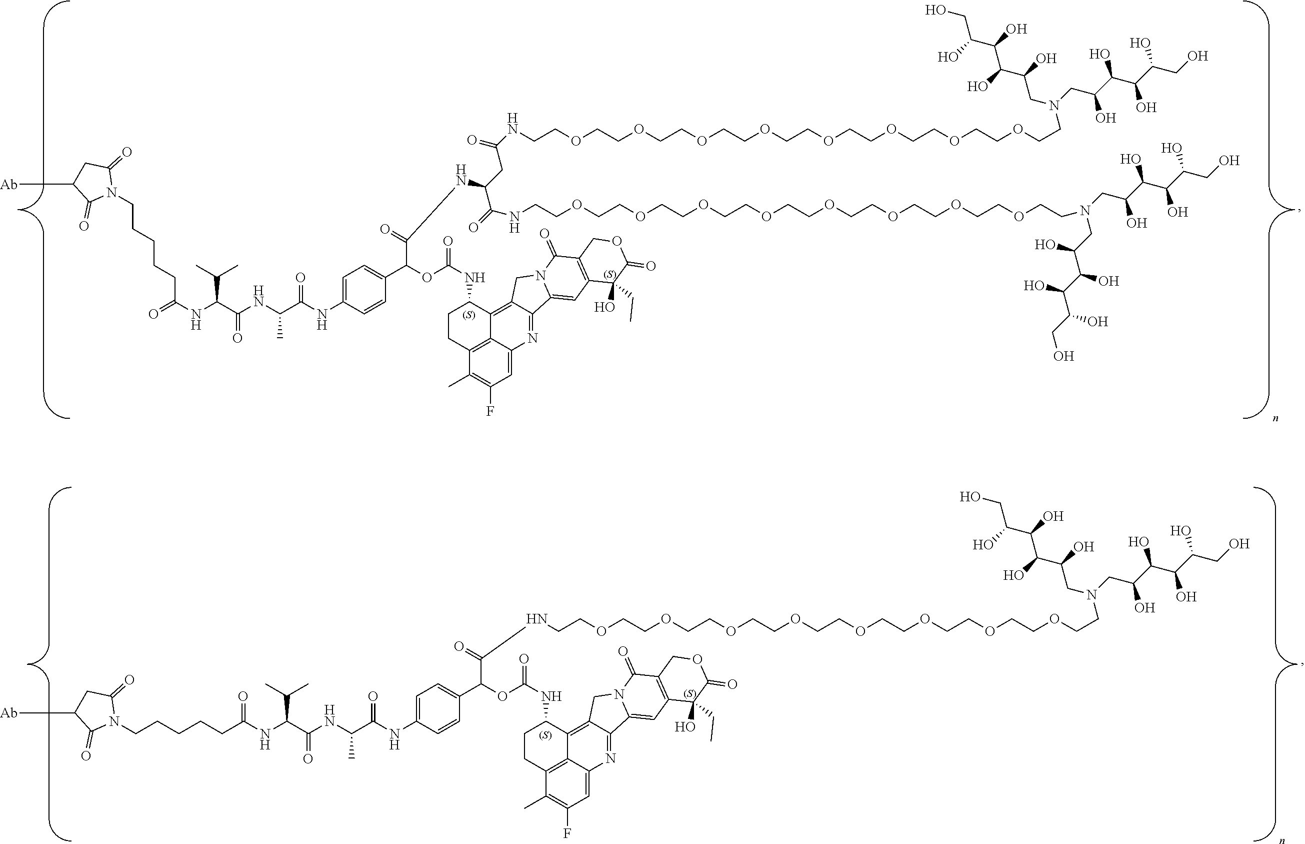

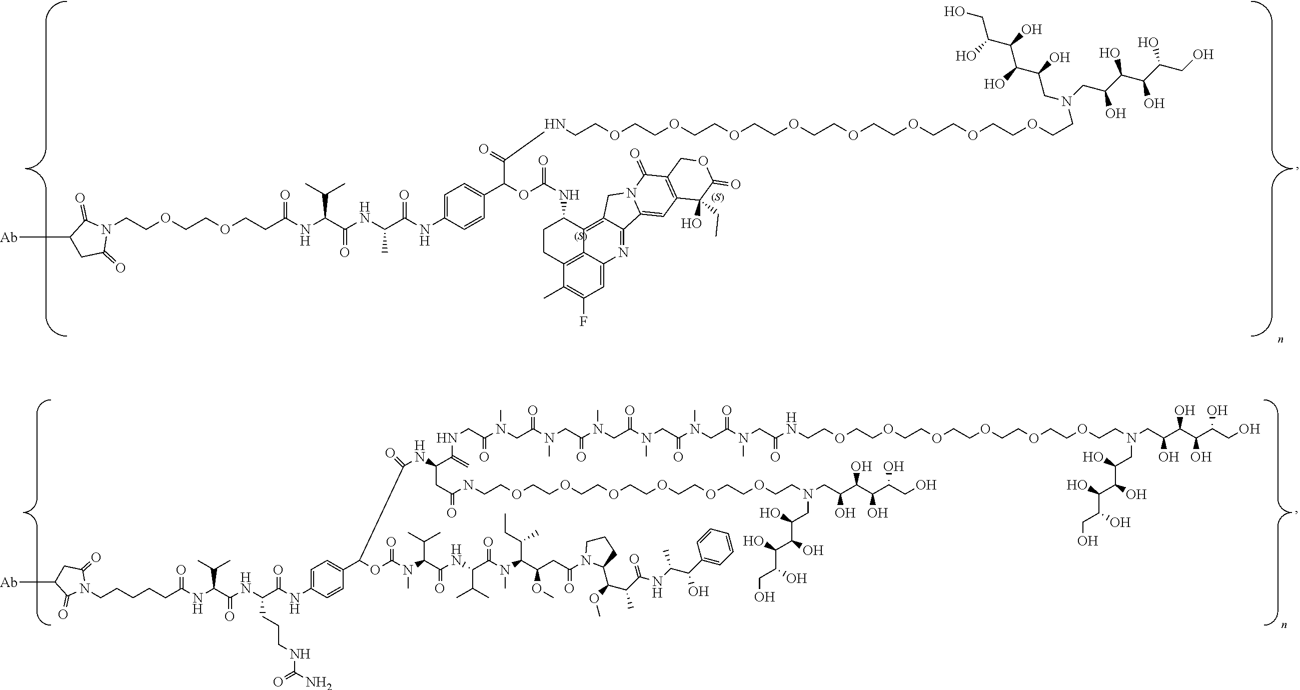

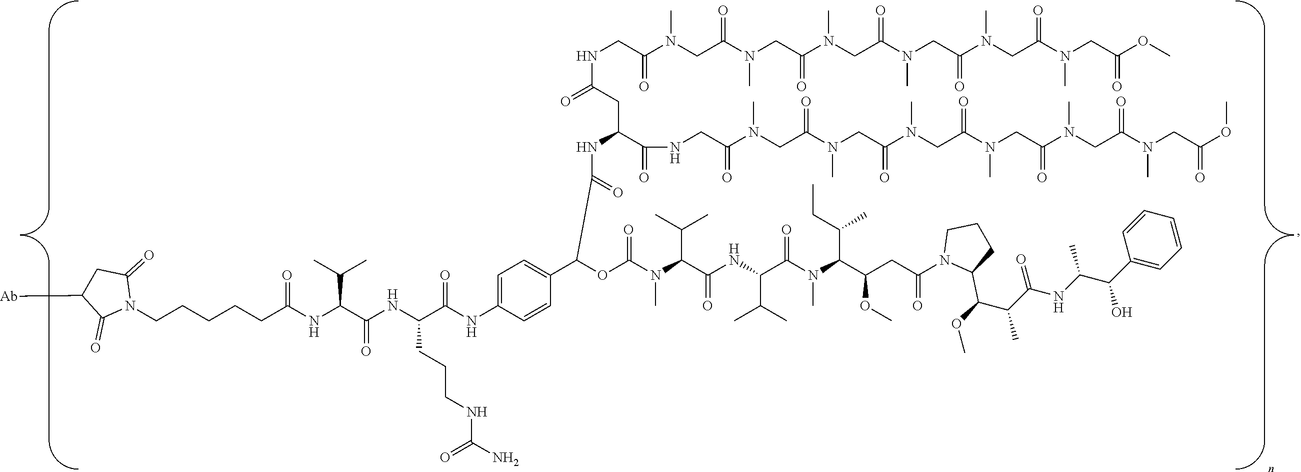

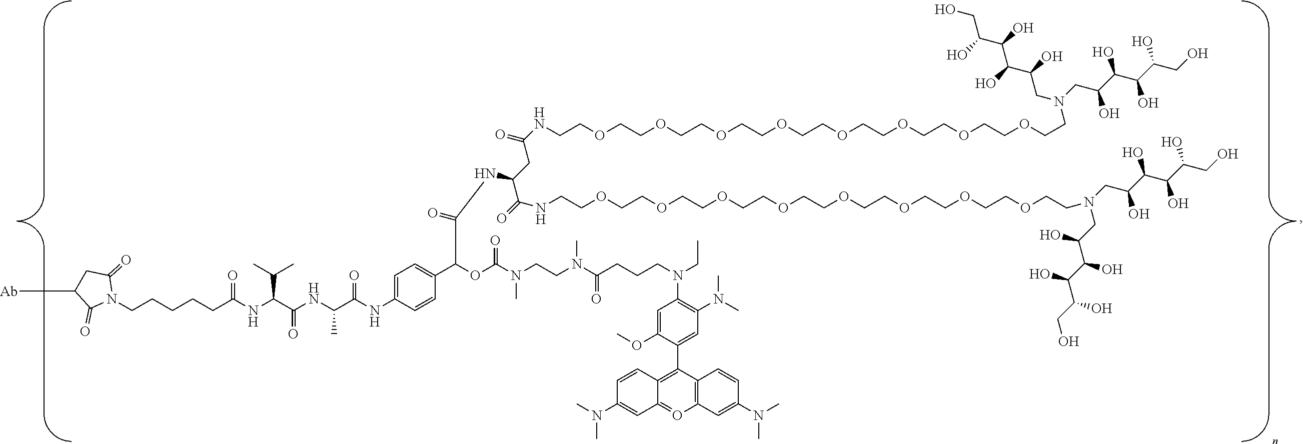

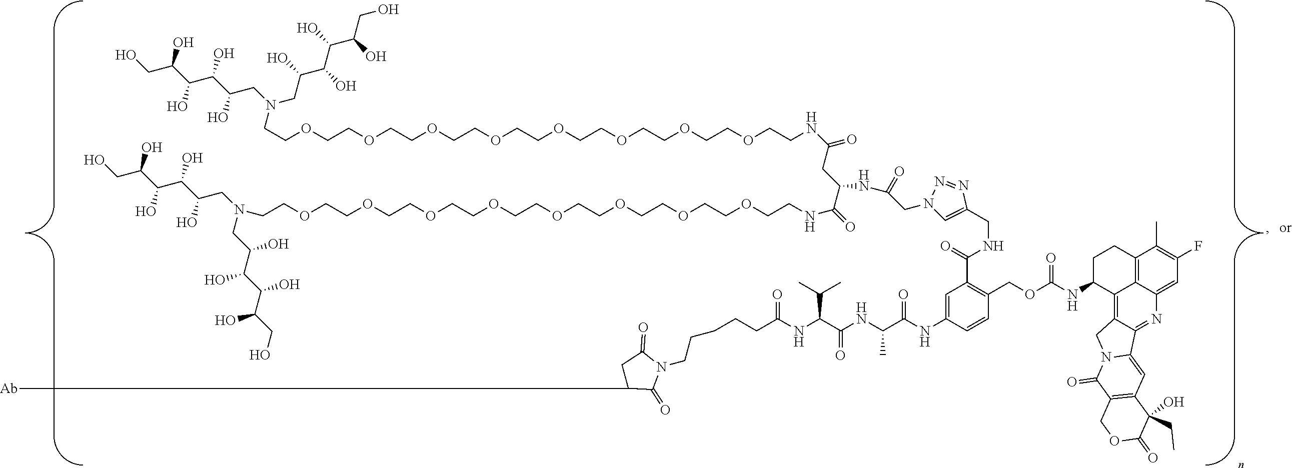

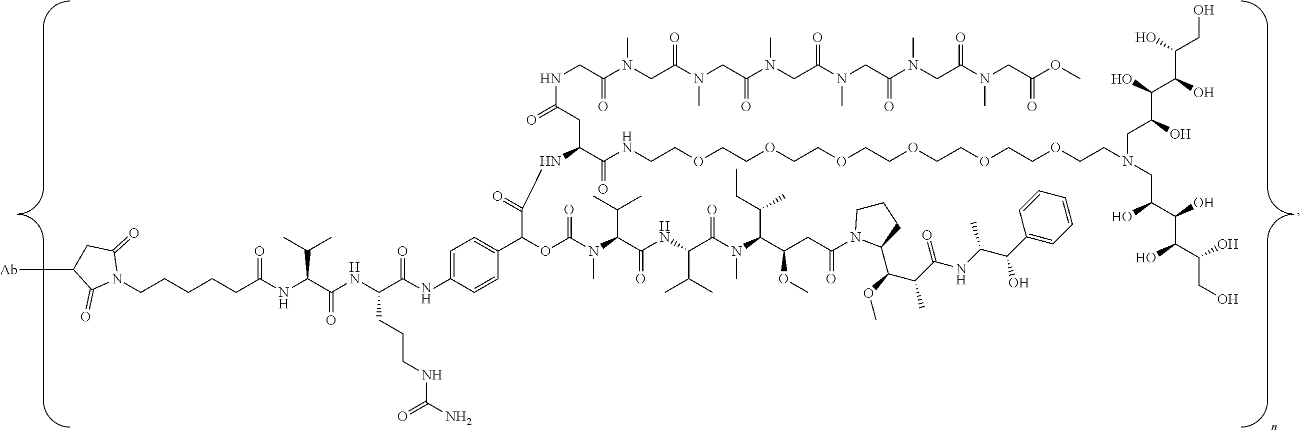

wherein Ab is a Targeting group and n is p^{load}.

Embodiment 118. The conjugate of any one of Embodiments 109, 116, or 117, wherein the Targeting group specifically binds to a target molecule.

Embodiment 119. The conjugate of Embodiment 118, wherein the target molecule is CD19, CD20, CD3a, CD33, CD70, LIV-1, or EGFRv3.

Embodiment 120. The conjugate of any one of Embodiments 109, 116, or 117, wherein the Targeting group is selected from: a scFv1-ScFv2, a ScFv12-Fc-scFv22, a IgG-scFv, a DVD-Ig, a triomab/quadroma, a two-in-one IgG, a scFv2-Fc, a TandAb, and an scFv-HSA-scFv.

Embodiment 121. The conjugate of any one of Embodiments 109, 116, or 117, wherein the target molecule is a cancer associated antigen.

Embodiment 122. The conjugate of any one of Embodiments 109, 116 or 117, wherein the target molecule is CD19, CD20, CD30, CD33, CD38, CA125, HER2, MUC-1, prostate-specific membrane antigen (PSMA), CD44 surface adhesion molecule, mesothelin (MLSN), carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-A1, IL-13R-a2, GD2, 1p19q, ABL1, AKT1, ALK, APC, AR, ATM, BRAF, BRCA1, BRCA2, cKIT, cMET, CSF1R, CTNNB1, FGFR1, FGFR2, FLT3, GNA11, GNAQ, GNAS, HRAS, IDH1, IDH2, JAK2, KDR (VEGFR2), KRAS, MGMT, MGMT-Me, MLH1, MPL, NOTCH1, NRAS, PDGFRA, Pgp, PIK3CA, PR, PTEN, RET, RRM1, SMO, SPARC, TLE3, TOP2A, TOPO1, TP53, TS, TUBB3, VHL, CDH1, ERBB4, FBXW7, HNF1A, JAK3, NPM1, PTPN11, RB1, SMAD4, SMARCB1, STK1, MLH1, MSH2, MSH6, PMS2, ROS1, ERCC1, 5T4 (TPBG), B7-H3, CCR7, CD105, CD22, CD46, CD47, CD56, CD70, CD71, CD79b, CDH6, CLDN6, CLDN18.2, CLEC12A, DLL3, DR5, ERBB3 (HER3), EPCAM, FOLR1, IGF1R, IL2RA (CD25), IL3RA, ITGB6, LIV-1, LRRC15, mesothelin (MSLN), NaPi2b (SLC34A2), nectin-4, PTK7, ROR1, SEZ6, SLC44A4, SLITRK6, Tissue Factor (TF), TROP2 or B7-H4.

Embodiment 123. The conjugate of any one of Embodiments 109, 116 or 117, wherein the Targeting group is an antibody, or fragment thereof, comprising rituximab (Rituxan®), trastuzumab (Herceptin®), pertuzumab (Perjeta®)), bevacizumab (Avastin®), ranibizumab (Lucentis®), cetuximab (Erbitux®), alemtuzumab (Campath®), panitumumab (Vectibix®), ibritumomab tiuxetan (Zevalin®), tositumomab (Bexxar®), ipilimumab, zalutumumab, dalotuzumab, figitumumab, ramucirumab, galiximab, farletuzumab, ocrelizumab, ofatumumab (Arzerra®), tositumumab, ibritumomab, the CD20 antibodies 2F2 (HuMax-CD20), 7D8, IgM2C6, IgG1 2C6, 11B8, B1, 2H7, LT20, 1FS or AT80, daclizumab (Zenapax®), or anti-LHRH receptor antibodies including clone A9E4, F1G4, AT2G7, GNRH03, or GNRHR2.

Embodiment 124. A pharmaceutical composition comprising the conjugate of any one of Embodiments 109 to 123 and a pharmaceutically acceptable carrier.

Embodiment 125. A method of treating a subject in need thereof, comprising administering to the subject a conjugate of any one of Embodiments 109 to 123 or the pharmaceutical composition of Embodiment 124, wherein the subject has cancer or an autoimmune disease and the conjugate binds to a target antigen associated with the cancer or autoimmune disease.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure. All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### EXAMPLES

### Abbreviations

Boc₂O: di-tert-butyl dicarbonate
Bu₄NBr: Tetrabutylammonium bromide
DCM: dichloromethane
DEA: Diethanolamine
DEAD: diethyl azodicarboxylate
DIPEA: N,N-diisopropylethylamine
DMAP: 4-(Dimethylamino)pyridine
DMF: N,N-dimethylformamide
DMTMM: 4-(4,6-dimethoxy-1,3,5-triazin-2-yl)-4-methyl-morpholinium chloride
DMSO: dimethylsulfoxide
EEDQ: N-Ethoxycarbonyl-2-ethoxy-1,2-dihydroquinoline
ESI: electrospray ionization
HATU: 1-[bis(dimethyamino)methylene]-1H-1,2,3-triazolo[4,5-b]pyridinium 3-oxidhexa fluorophosophate)
HOBt: hydroxylbenzotriazole
LCMS: liquid chromatography-mass spectrometry
MeCN: acetonitrile
MeOH: methanol
m-CPBA: meta-chloroperoxybenzoic acid
MTBE: Methyl tert-butyl ether
NMR: nuclear magnetic resonance spectroscopy
Ph₃CCl: Triphenylmethyl chloride
PNPC: bis(4-nitrophenyl) carbonate
PPh₃: triphenylphosphine
TFA: trifluoroacetic acid
THF: tetrahydrofuran
TLC: thin-layer chromatography
TsOH: p-Toluenesulfonic acid

### General Methods

¹H NMR and other NMR spectra were recorded on Bruker AVIII 400 or Bruker AVIII 500. The data were processed with Nuts software or MestReNova software, measuring proton shifts in parts per million (ppm) downfield from an internal standard tetramethyl silane.

HPLC-MS measurement was run on Agilent 1200 HPLC/6100 SQ System using the following conditions:

Method A: Mobile Phase: A: Water (0.01% TFA) B: acetonitrile (0.01% TFA); Gradient Phase: 5% of B increasing to 95% of B in 15 min; Flow Rate: 1.0 mL/min; Column: XBridge C18, 4.6*150 mm, 3.5 μm; Column Temperature: 40° C. Detectors: ADC ELSD, DAD (214 nm and 254 nm), ES-API.

Method B: Mobile Phase: A: Water (0.01% TFA) B: acetonitrile (0.01% TFA); Gradient Phase: 5% of B increasing to 95% of B in 15 min; Flow Rate: 1.0 mL/min; Column: SunFire C18, 4.6*150 mm, 3.5 μm; Column Temperature: 45° C. Detectors: ADC ELSD, DAD (214 nm and 254 nm), ES-API.

Method C: Mobile Phase: A: Water (10 mM NH₄HCO₃) B: acetonitrile; Gradient Phase: 5% to 95% of B in 15 min; Flow Rate: 1.0 mL/min; Column: XBridge C18, 4.6*150 mm, 3.5 μm; Column Temperature: 40° C. Detectors: ADC ELSD, DAD (214 nm and 254 nm), MSD (ES-API).

LCMS measurement was run on Agilent 1200 HPLC/6100 SQ System using the following conditions:

Method A: Mobile Phase: A: Water (0.01% TFA) B: acetonitrile (0.01% TFA); Gradient Phase: 5% of B increasing to 95% of B in 3 min; Flow Rate: 1.8-2.3 mL/min; Column: SunFire C18, 4.6*50 mm, 3.5 μm; Column Temperature: 50° C. Detectors: ADC ELSD, DAD (214 nm and 254 nm), ES-API.

Method B: Mobile Phase: A: Water (10 mM NH₄HCO₃) B: Acetonitrile; Gradient Phase: 5% to 95% of B in 3 min; Flow Rate: 1.8-2.3 mL/min; Column: XBridge C18, 4.6*50 mm, 3.5 μm; Column Temperature: 50° C. Detectors: ADC ELSD, DAD (214 nm and 254 nm), MSD (ES-API).

Preparative high pressure liquid chromatography (Prep-HPLC) was run on Gilson 281 using the following conditions:

Method A: Waters SunFire 10 μm C18 column (100 Å, 250×19 mm). Solvent A was water/0.01% trifluoroacetic acid (TFA) and solvent B was acetonitrile. The elution condition was a linear gradient increase of solvent B from 5% to 100% over a time period of 20 minutes at a flow rate of 30 mL/min.

Method B: Waters SunFire 10 μm C18 column (100 Å, 250×19 mm). Solvent A was water/0.05% formic acid (FA) and solvent B was acetonitrile. The elution condition was a linear gradient increase of solvent B from 5% to 100% over a time period of 20 minutes at a flow rate of 30 mL/min.

Method C: Waters Xbridge 10 μm C18 column (100 Å, 250×19 mm). Solvent A was water/10 mM ammonium bicarbonate (NH₄HCO₃) and solvent B was acetonitrile. The elution condition was a linear gradient increase of solvent B from 5% to 100% over a time period of 20 minutes at a flow rate of 30 mL/min.

Flash chromatography was performed on instrument of Biotage, with Agela Flash Column silica-CS; Reverse phase flash chromatography was performed on instrument of Biotage, with Boston ODS or Agela C18.

### Example 1: Preparation of Drug-Linker 1

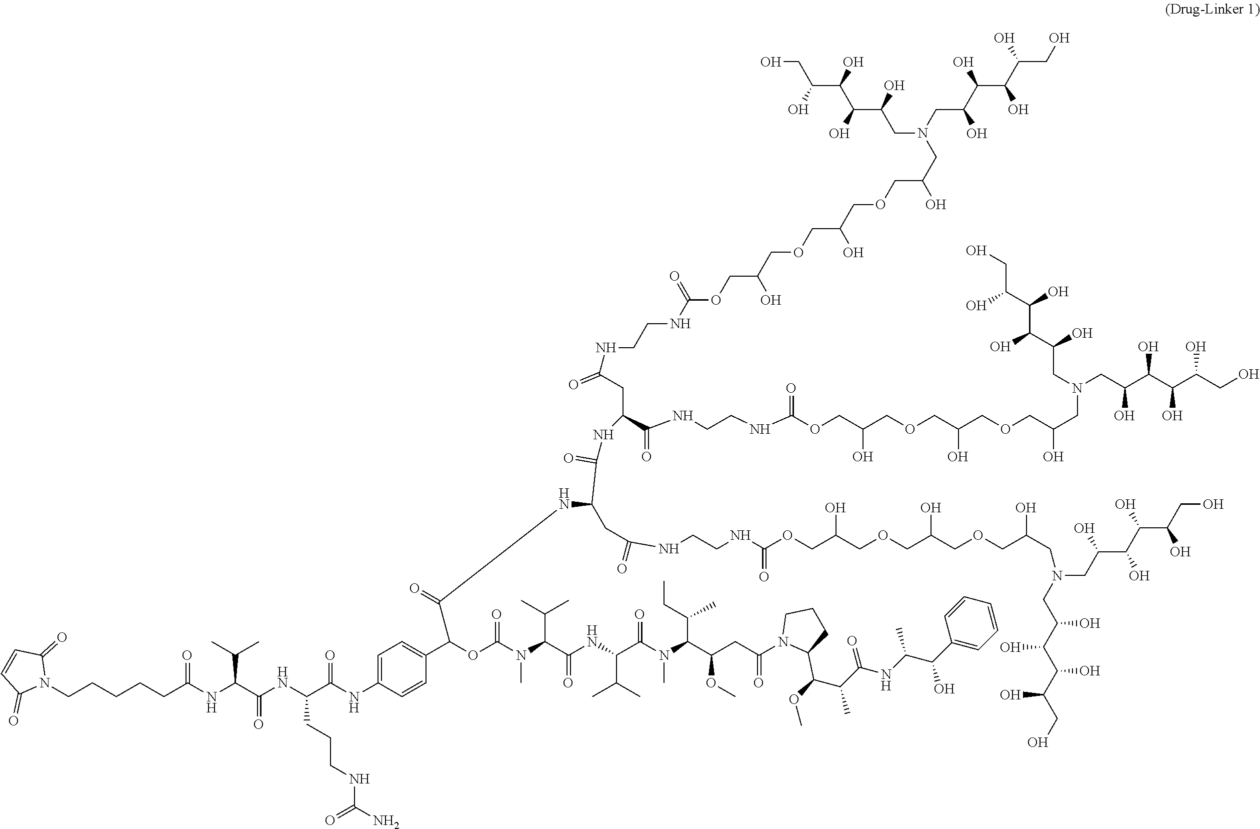

### Step 1

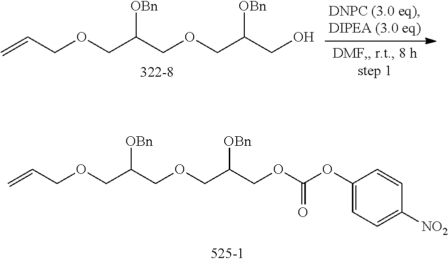 [SMILES: C=CCOCC(COCC(CO)OCc1ccccc1)OCc1ccccc1 ; C=CCOCC(COCC(COC(=O)Oc1ccc([N+](=O)[O-])cc1)OCc1ccccc1)OCc1ccccc1]

To a solution of 322-8 (600 mg, 1.554 mmol) in DMF (12 mL) was added DIPEA (602.4 mg, 4.661 mmol), followed by 4,4′-dinitrodiphenyl carbonate (1.42 g, 4.661 mmol), then the resulting mixture was stirred at room temperature for 8 hrs until 322-8 was consumed as detected by LCMS. The reaction solution was directly used in the next step without a work-up procedure.

### Step 2

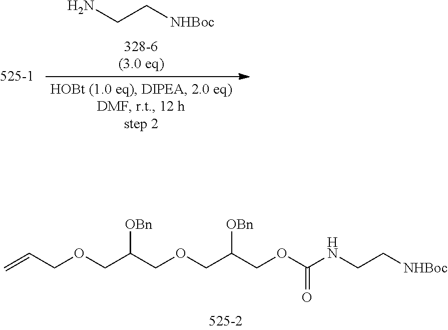 [SMILES: CC(C)(C)OC(=O)NCCN ; C=CCOCC(COCC(COC(=O)NCCNC(=O)OC(C)(C)C)OCc1ccccc1)OCc1ccccc1]

To the above reaction mixture was added HOBt (210 mg, 1.554 mmol), DIPEA (401.7 mg, 3.108 mmol) and 328-6 (746.5 mg, 4.662 mmol) successively, and the resulting mixture was stirred at room temperature for 6 hrs until 525-1 was consumed as detected by LCMS. The reaction mixture was diluted with ethyl acetate (180 mL) and washed with saturated NaHCO₃ (aq, 45 mL×3), dried over anhydrous Na₂SO₄, filtered and concentrated to dryness under reduced pressure. The crude product was purified with column chromatography (silica, 0-80% ethyl acetate in petroleum ether) affording 525-2 (893 mg, 1.56 mmol, 100.4% over 2 steps) as a pale yellow oil.

### Step 3

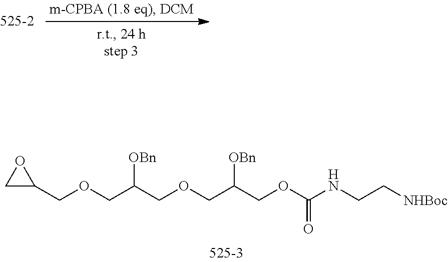 [SMILES: CC(C)(C)OC(=O)NCCNC(=O)OCC(COCC(COCC1CO1)OCc1ccccc1)OCc1ccccc1]

To a solution of 525-2 (890 mg, 1.555 mmol) in DCM (10 mL) was added a solution of m-CPBA (483 mg, 2.80 mmol) in DCM (10 mL) dropwise at room temperature. The resulting mixture was stirred at this temperature for 24 hours until 525-2 was consumed, and the reaction was quenched with saturated Na₂S₂O₃ (aq, 10 mL) and NaHCO₃ (aq., 10 mL). The reaction mixture was stirred for 30 mins, and then diluted with DCM (50 mL). The organic phase was washed with a mixture solution (20 mL×3) of saturated Na₂S₂O₃ and NaHCO₃ (aq, 1:1, V/V), dried over anhydrous Na₂SO₄, filtered and concentrated to dryness under reduced pressure to give the crude product, which was purified with column chromatography (silica, 0-100% ethyl acetate in petroleum ether) affording 525-3 (790 mg, 1.343 mmol, 86.4%) as a pale yellow oil. Purity=90%-95%.

### Step 4

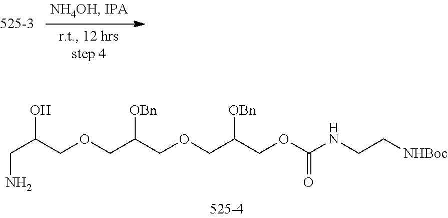 [SMILES: CC(C)(C)OC(=O)NCCNC(=O)OCC(COCC(COCC(O)CN)OCc1ccccc1)OCc1ccccc1]

To a solution of 525-3 (790 mg, 1.343 mmol) in isopropyl alcohol (110 mL) was added ammonia (110 mL) dropwise at room temperature, and the resulting mixture was stirred at this temperature for 12 hours until 525-3 was consumed. The reaction mixture was concentrated to dryness under reduced pressure to afford 525-4 (801.2 mg, 1.323 mmol, 98.6%) as a yellow oil, which was used in the next step without further purification. Purity=90%-95%.

### Step 5

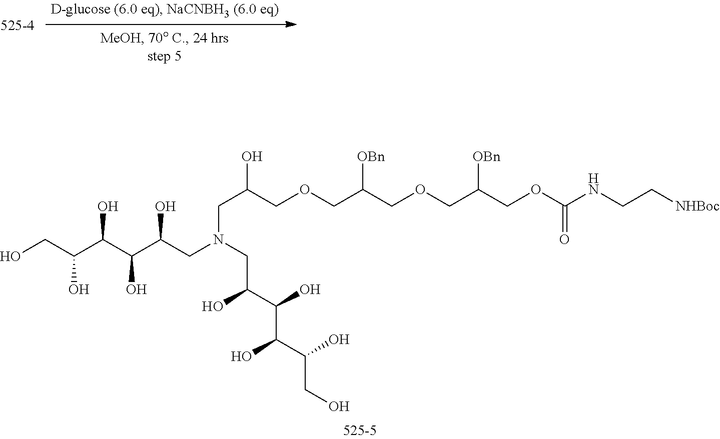 [SMILES: CC(C)(C)OC(=O)NCCNC(=O)OCC(COCC(COCC(O)CN(C[C@H](O)[C@@H](O)[C@H](O)[C@H](O)CO)C[C@H](O)[C@@H](O)[C@H](O)[C@H](O)CO)OCc1ccccc1)OCc1ccccc1]

A mixture of 525-4 (801.2 mg, 1.324 mmol), D-glucose (1.43 g, 7.937 mmol) and NaCNBH₃ (499.2 mg, 7.94 mmol) in anhydrous MeOH (21 mL) was stirred at 70° C. for 24 hrs until most of 525-4 was consumed and 525-5 was detected by LCMS. The reaction mixture was cooled down to room temperature, filtered and concentrated under reduced pressure to give the crude product, which was purified by reverse phase liquid chromatography to give 525-5 (1.2 g, 1.29 mmol, 97.1%) as a colorless oil. Purity=85%-90%.

### Step 6

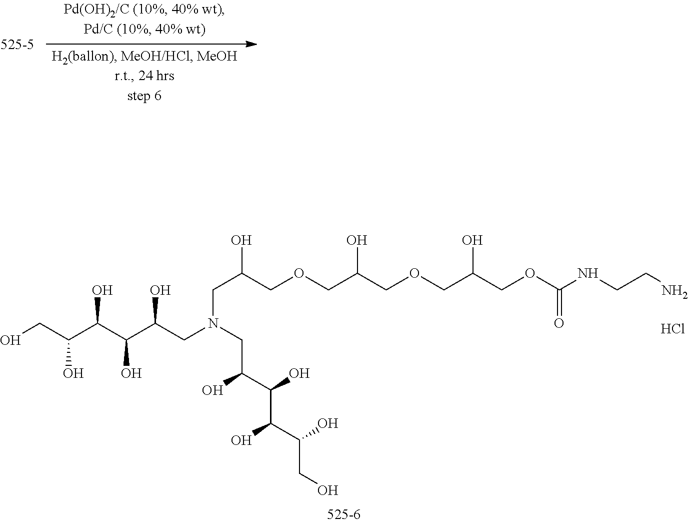 [SMILES: NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO ; Cl]

A mixture of 525-5 (1.2 g, 1.286 mmol), Pd(OH)₂/C (10%, 250 mg) and Pd/C (10%, 250 mg) in HCl/MeOH (4M, 25 mL), MeOH (25 mL) was stirred under hydrogen atmosphere (balloon) for 24 h at room temperature until 525-5 was completely converted into 525-6. The reaction mixture was filtered through a celite pad, and the filtrate was concentrated to dryness under reduced pressure to afford 525-6 (886 mg, 1.285 mmol, 100.0%) as an off-white solid, which was used in the next step without further purification.

### Step 7

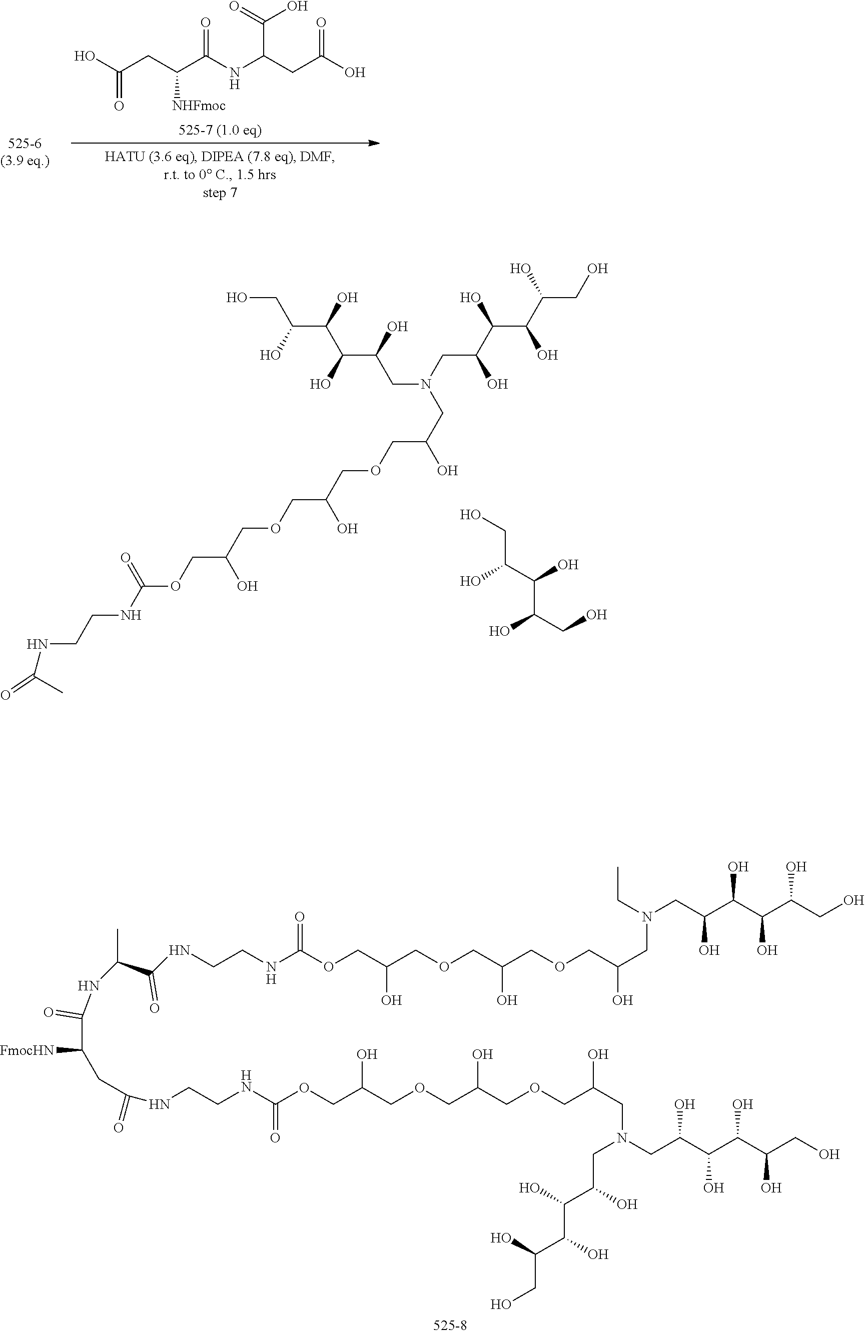

A solution of 525-7 (104.9 mg, 0.223 mmol) and HATU (305.4 mg, 0.803 mmol) in anhydrous DMF (3 mL) was stirred at room temperature for 15 mins, then it was stirred in an ice bath. A solution of 525-6 (600 mg, 0.870 mmol) in anhydrous DMF (3 mL) was added dropwise, followed by DIPEA (225 mg, 1.74 mmol). The resulting mixture was stirred in the ice bath for 1 h until most of 526-7 was consumed. The reaction mixture was purified by reverse phase liquid chromatography to give 525-8 (269.8 mg, 0.113 mmol, 50.9%) as a white solid. Purity=90%-95%. 1H NMR (400 MHz, DMSO-d₆) δ 7.87 (d, J=7.6 Hz, 2H), 7.64 (d, J=7.2 Hz, 2H), 7.47 (t, J=7.6 Hz, 2H), 7.42-7.32 (m, 2H), 4.65-4.57 (m, 1H), 4.53-4.36 (m, 3H), 4.33-4.13 (m, 10H), 4.08-3.87 (m, 12H), 3.84-3.69 (m, 18H), 3.67-3.35 (m, 53H), 3.30-3.07 (m, 12H), 2.77-2.32 (m, 4H).

### Step 8

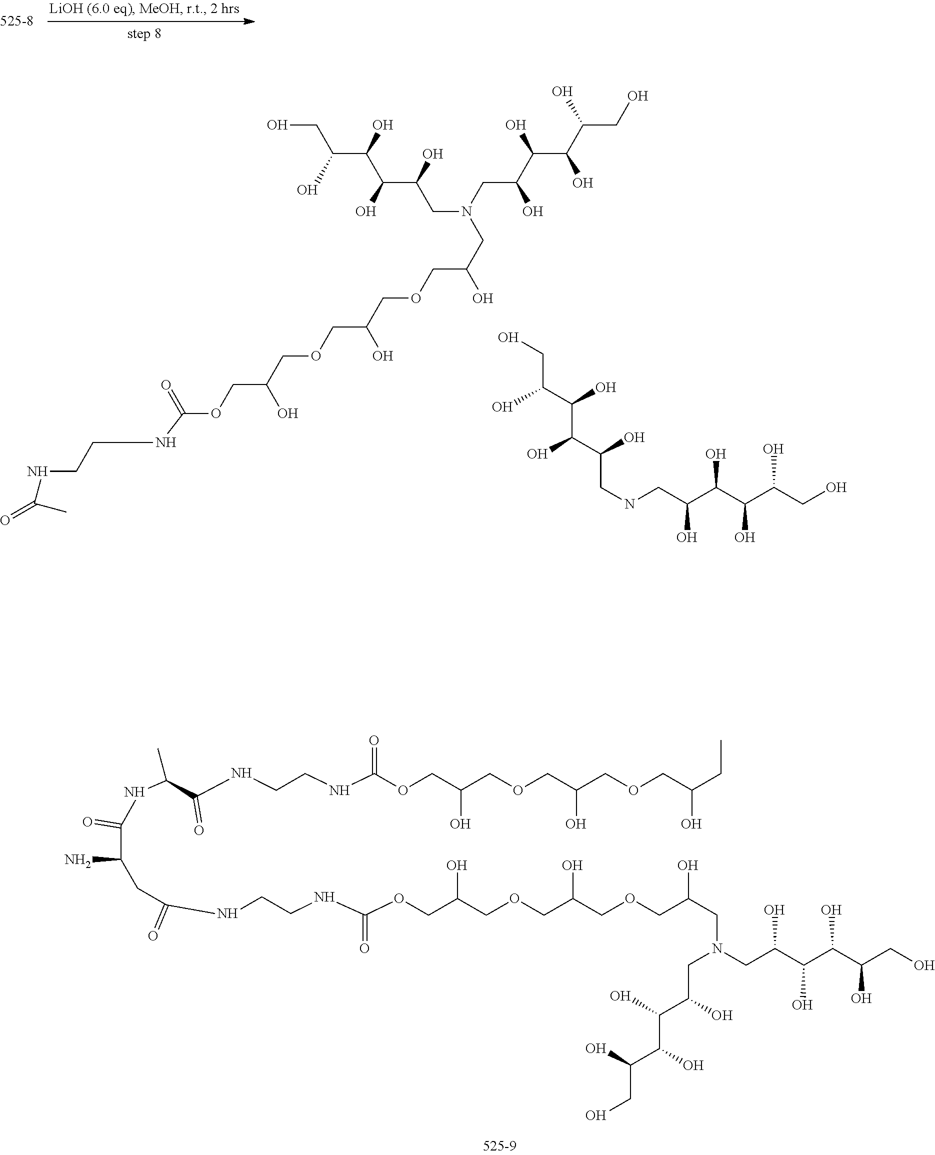 [SMILES: CC(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO ; OC[C@@H](O)[C@@H](O)[C@H](O)[C@@H](O)CNC[C@H](O)[C@@H](O)[C@H](O)[C@H](O)CO ; CCC(O)COCC(O)COCC(O)COC(=O)NCCNC(=O)[C@H](C)NC(=O)[C@H](N)CC(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO]

To a solution of 525-8 (100 mg, 0.0421 mmol) in MeOH (3 mL) and H₂O (1 mL) was added LiOH·H₂O (10.6 mg, 0.252 mmol), and the mixture was stirred at room temperature for 2 hrs until 525-8 was consumed. The reaction solution was neutralized with 1N HCl to pH=7, and concentrated under reduced pressure to give a crude product, which was dissolved in H₂O (15 mL) and washed with hexane (10 mL×3). The aqueous phase was concentrated to dryness under reduced pressure to afford 525-9 (74.6 mg, 0.0346 mmol, 82.3%) as a colorless oil, which was used in the next step without further purification.

### Step 9

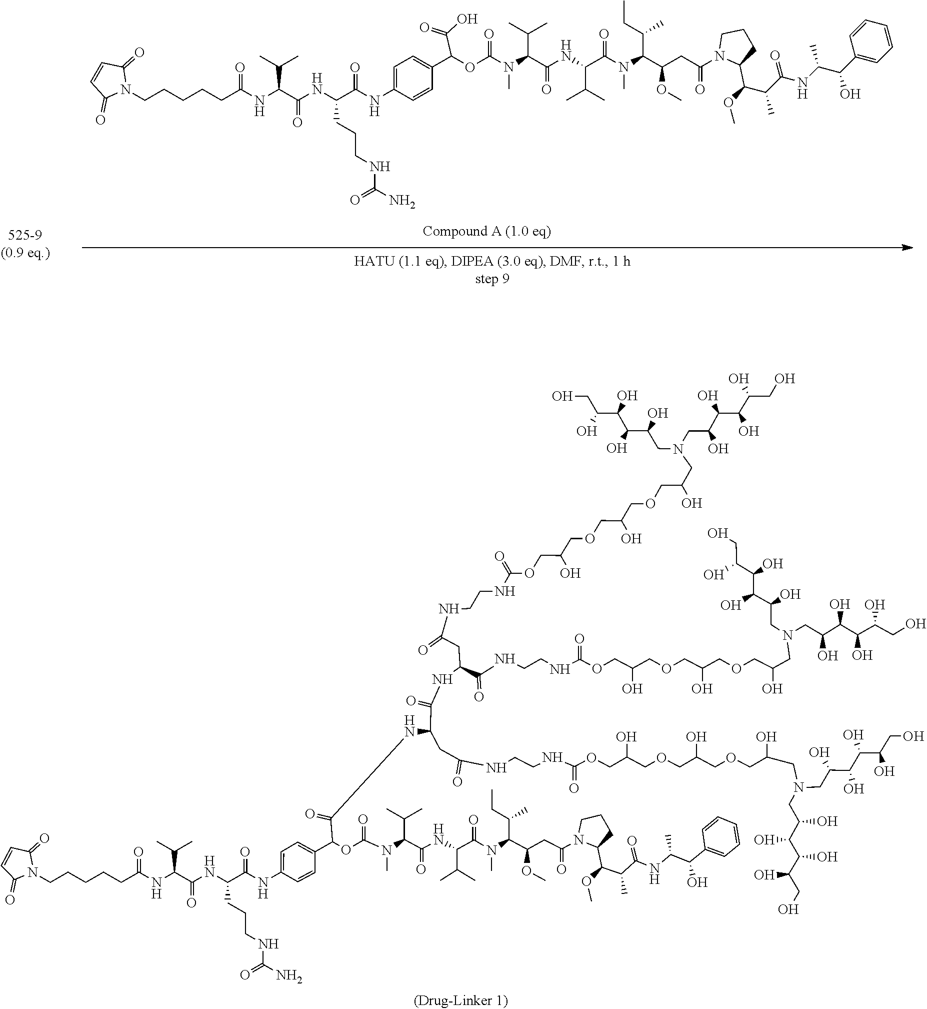

A solution of 525-9 (70.0 mg, 0.0325 mmol), Compound A (49 mg, 0.0360 mmol), HATU (15.1 mg, 0.0397 mmol) and DIPEA (14.0 mg, 0.108 mmol) in anhydrous DMF (4 mL) was stirred at room temperature for 1 h until Compound A was consumed. Then the reaction solution was purified by prep-HPLC to give Drug-Linker 1 (23.5 mg, 0.00672 mmol, 20.7%) as a white solid. LCMS, m/z=1749.66 (M/2+H)+, m/z=1166.72 (M/2+H)⁺. ¹H NMR (400 MHz, DMSO-d₆) δ 7.61-7.40 (m, 4H), 7.39-7.26 (m, 4H), 7.25-7.13 (m, 1H), 6.77 (s, 2H), 5.98 (t, J=13.6 Hz, 1H), 4.75-4.52 (m, 7H), 4.49-4.38 (m, 2H), 4.37-3.90 (m, 25H), 3.87-3.38 (m, 73H), 3.37-2.87 (m, 28H), 2.87-2.08 (m, 12H), 2.07-1.95 (m, 2H), 1.93-1.65 (m, 5H), 1.68-1.41 (m, 8H), 1.37-1.11 (m, 8H), 1.09-1.02 (m, 2H), 1.00-0.75 (m, 20H), 0.74-0.67 (m, 1H), 0.57-0.48 (m, 1H), 0.33 (d, J=6.4 Hz, 1H).

### Example 2: Preparation of Drug-Linker 2

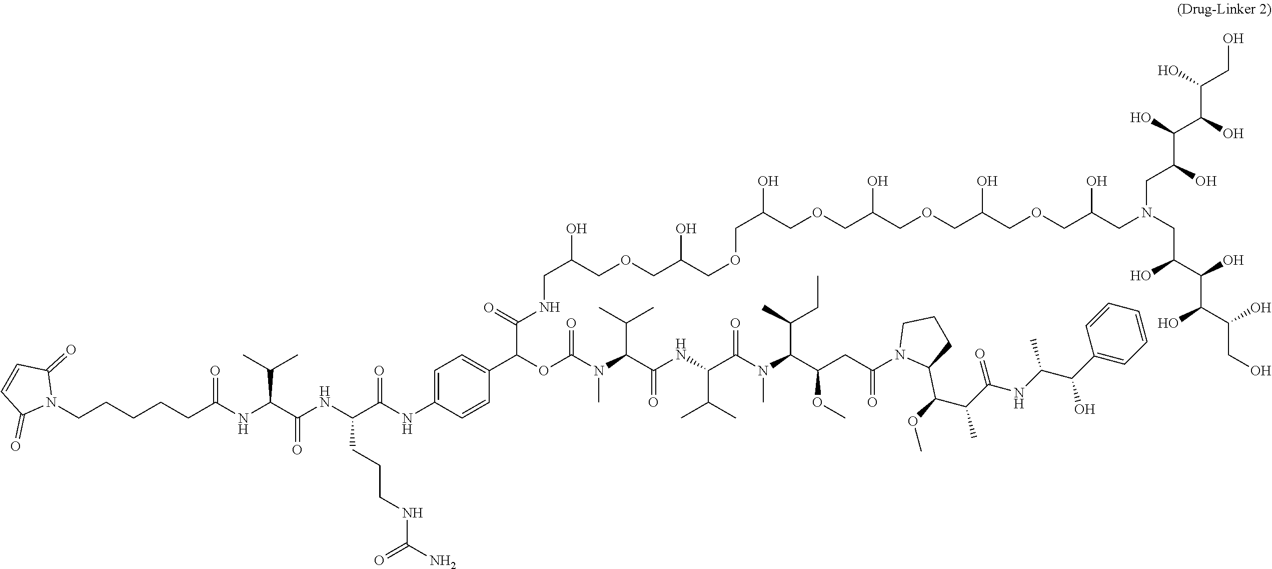 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

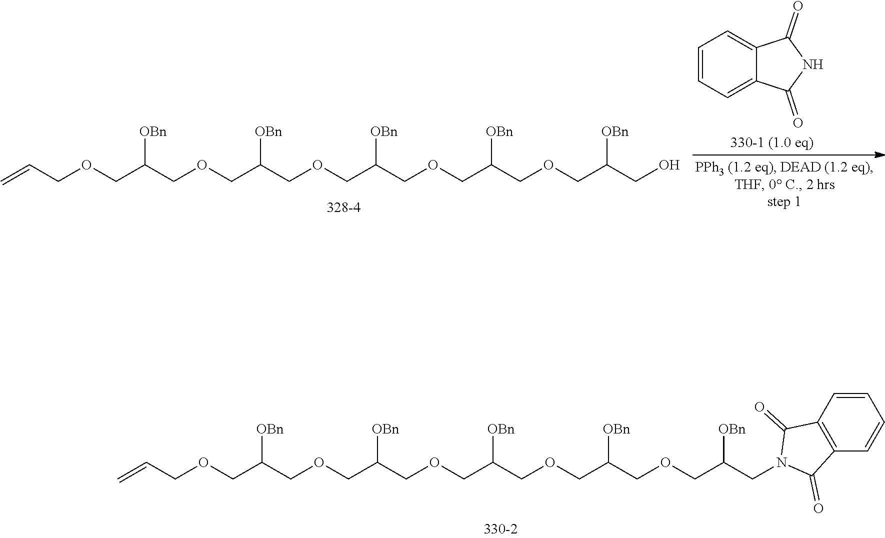 [SMILES: O=C1NC(=O)c2ccccc21 ; C=CCOCC(COCC(COCC(COCC(COCC(CO)OCc1ccccc1)OCc1ccccc1)OCc1ccccc1)OCc1ccccc1)OCc1ccccc1 ; C=CCOCC(COCC(COCC(COCC(COCC(CN1C(=O)c2ccccc2C1=O)OCc1ccccc1)OCc1ccccc1)OCc1ccccc1)OCc1ccccc1)OCc1ccccc1]

To a solution of 330-1 (217.6 mg, 1.48 mmol) and PPh₃ (465.5 mg, 1.776 mmol) in THF (8 mL) was added a solution of 328-4 (1.3 g, 1.48 mmol) in THF (4 mL) and the mixture was stirred in an ice bath. A solution of DEAD (309.3 mg, 1.776 mmol) in THF (1 mL) was added to the above solution and the resulting mixture was allowed to warm to r.t. and stirred for 2 hrs until 328-4 was consumed by TLC. The reaction was quenched with water (1 mL), and the reaction was concentrated under reduced pressure to give the crude product, which was purified with column chromatography (silica, 0-60% ethyl acetate in petroleum ether) to afford 330-2 (1.307 g, 1.297 mmol, 87.7%) as a white solid. Purity=90%-95%. 1H NMR (400 MHz, CDCl₃) δ 7.77 (dd, J=5.6, 3.2 Hz, 2H), 7.66 (dd, J=5.6, 3.2 Hz, 2H), 7.34-7.20 (m, 20H), 7.19-7.15 (m, 2H), 7.12-7.04 (m, 3H), 5.95-5.81 (m, 1H), 5.29-5.22 (m, 1H), 5.15 (d, J=10.4 Hz, 1H), 4.69-4.63 (m, 9H), 4.51 (d, J=12.0 Hz, 1H), 3.99-3.95 (m, 2H), 3.93-3.83 (m, 2H), 3.76-3.69 (m, 5H), 3.60-3.52 (m, 18H).

### Step 2

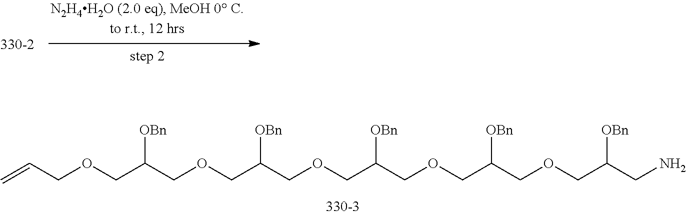 [SMILES: C=CCOCC(COCC(COCC(COCC(COCC(CN)OCc1ccccc1)OCc1ccccc1)OCc1ccccc1)OCc1ccccc1)OCc1ccccc1]

To a solution of 330-2 (1.472 g, 1.46 mmol) in MeOH (10 mL) was added N₂H₄·H₂O (146.3 mg, 2.92 mmol) in an ice bath, the resulting mixture was allowed to warm to r.t. and stirred for 10 hrs until 330-2 was consumed by TLC. The reaction mixture was concentrated to dryness under reduced pressure to give the crude product, which was dissolved in ethyl acetate (20 mL) and filtered. The filtrate was concentrated under reduced pressure to afford 330-3 (1.23 g, 1.402 mmol, 95.9%) as a colorless oil, which was used in the next step without further purification.

### Step 3

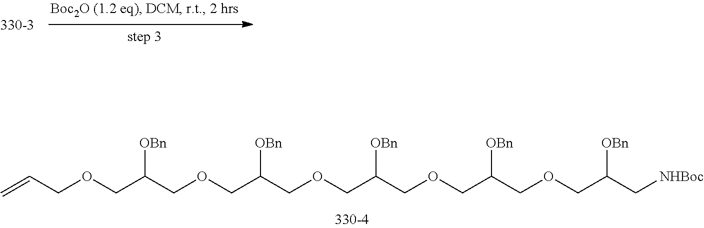 [SMILES: C=CCOCC(COCC(COCC(COCC(COCC(CNC(=O)OC(C)(C)C)OCc1ccccc1)OCc1ccccc1)OCc1ccccc1)OCc1ccccc1)OCc1ccccc1]

To a solution of 330-3 (1.23 g, 1.40 mmol) in DCM (8 mL) was added Boc₂O (367 mg. 1.68 mmol) at room temperature, and the reaction mixture was stirred at this temperature for 2 hrs until 330-3 was consumed and 330-4 was detected by LCMS. The reaction was concentrated under reduced pressure to give the crude product, which was purified with column chromatography (silica, 0-40% ethyl acetate in petroleum ether) affording 330-4 (1.23 g, 1.26 mmol, 89.8%) as a colorless oil. Purity=90%-95%.

### Step 4

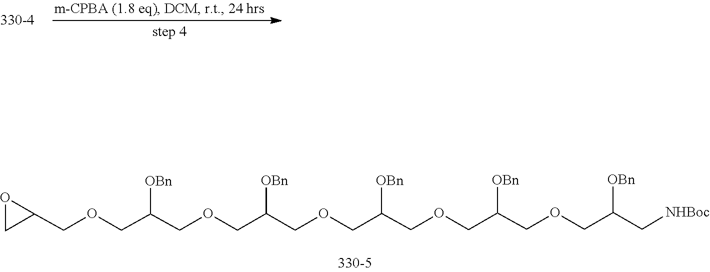 [SMILES: CC(C)(C)OC(=O)NCC(COCC(COCC(COCC(COCC(COCC1CO1)OCc1ccccc1)OCc1ccccc1)OCc1ccccc1)OCc1ccccc1)OCc1ccccc1]

To a solution of 330-4 (800 mg, 0.818 mmol) in DCM (5 mL) was added a solution of m-CPBA (254 mg, 1.473 mmol) in DCM (5 mL) at room-temperature, and the mixture was stirred for 24 hrs until 330-4 was consumed by TLC. The reaction was quenched by adding sat. Na₂S₂O₃ (5 mL) and sat. NaHCO₃ (5 mL), and the resulting mixture was stirred for 30 mins before diluting with DCM (60 mL). The organic phase was sequentially washed with sat. Na₂S₂O₃ (20 mL) and sat. NaHCO₃ (20 mL), dried over anhydrous Na₂SO₄, filtered and concentrated to dryness under reduced pressure. The crude product was purified with column chromatography (silica, 0-60% Ethyl acetate in petroleum ether) to afford 330-5 (689 mg, 0.693 mmol, 84.8%) as a colorless oil. Purity=90%-95%.

### Step 5

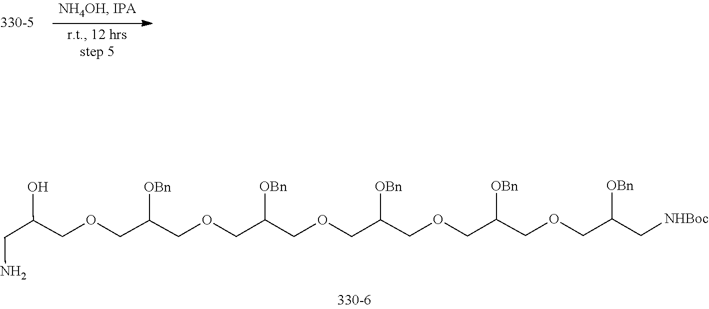 [SMILES: CC(C)(C)OC(=O)NCC(COCC(COCC(COCC(COCC(COCC(O)CN)OCc1ccccc1)OCc1ccccc1)OCc1ccccc1)OCc1ccccc1)OCc1ccccc1]

To a solution of 330-5 (689 mg, 0.693 mmol) in isopropyl alcohol (56 mL) was added ammonia (43 mL) dropwise at room temperature, and the resulting mixture was stirred at this temperature for 12 hours until 330-5 was consumed. The reaction mixture was concentrated to dryness under reduced pressure to afford 330-6 (698.7 mg, 0.691 mmol, 99.7%) as a yellow oil, which was used in the next step without further purification. Purity=90%-95%.

### Step 6

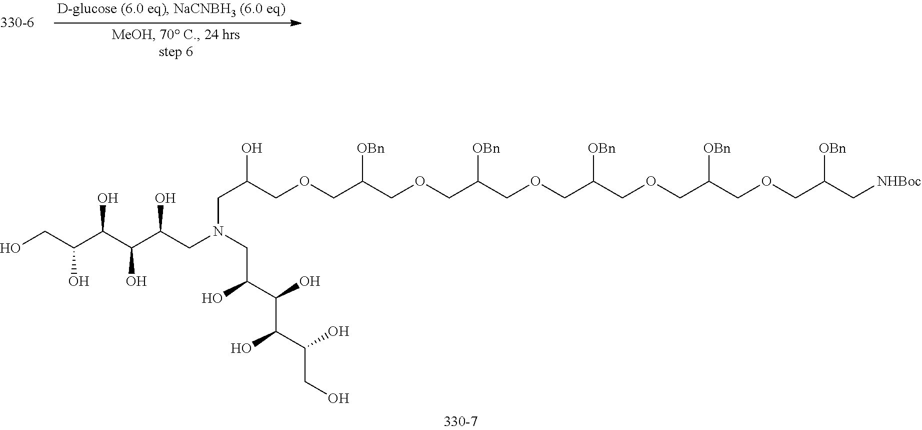 [SMILES: CC(C)(C)OC(=O)NCC(COCC(COCC(COCC(COCC(COCC(O)CN(C[C@H](O)[C@@H](O)[C@H](O)[C@H](O)CO)C[C@H](O)[C@@H](O)[C@H](O)[C@H](O)CO)OCc1ccccc1)OCc1ccccc1)OCc1ccccc1)OCc1ccccc1)OCc1ccccc1]

A mixture of 330-6 (983 mg, 0.973 mmol), D-glucose (1.05 g, 5.836 mmol) and NaCNBH₃ (366.7 mg, 5.836 mmol) in anhydrous MeOH (15 mL) was stirred at 70° C. for 24 hrs until most of 330-6 was consumed and 330-7 was detected by LCMS. The reaction mixture was cooled down to room temperature, filtered and concentrated under reduced pressure to give the crude product, which was purified by reverse phase liquid chromatography to give 330-7 (889 mg, 0.664 mmol, 68.2%) as a colorless oil. Purity=90%-95%.

### Step 7

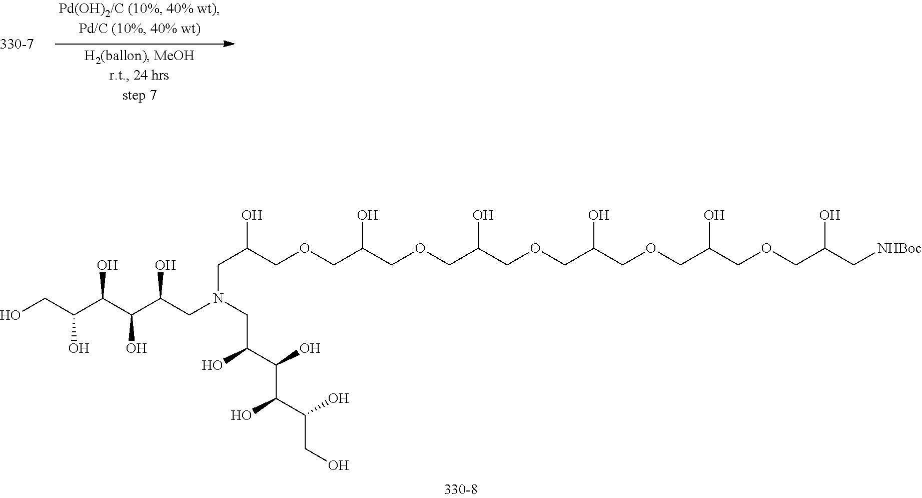 [SMILES: CC(C)(C)OC(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO]

A mixture of 330-7 (270 mg, 0.202 mmol), Pd(OH)₂/C (10%, 120 mg) and Pd/C (10%, 120 mg) in MeOH (25 mL) was stirred under hydrogen atmosphere (ballon) for 24 h at room temperature until 330-7 was completely converted into 330-8. The reaction mixture was filtered through a celite pad, and the filtrate was concentrated to dryness under reduced pressure to afford 330-8 (138.2 mg, 0.156 mmol, 77.0%) as an off-white solid, which was used in the next step without further purification.

### Step 8

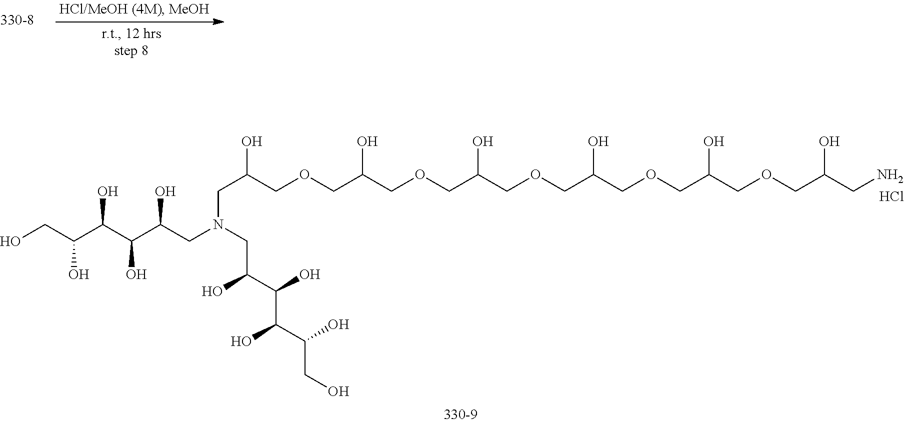 [SMILES: NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO ; Cl]

A solution of 330-8 (138 mg, 0.155 mmol) in HCl/MeOH (4M, 3 mL) and MeOH (3 mL) was stirred at room temperature for 12 hours until 330-8 was completely converted into 330-9. The reaction mixture was concentrated to dryness under reduced pressure to afford 330-9 (128 mg, 0.155 mmol, 100%) as an off-white solid, which was used in the next step without further purification.

### Step 9

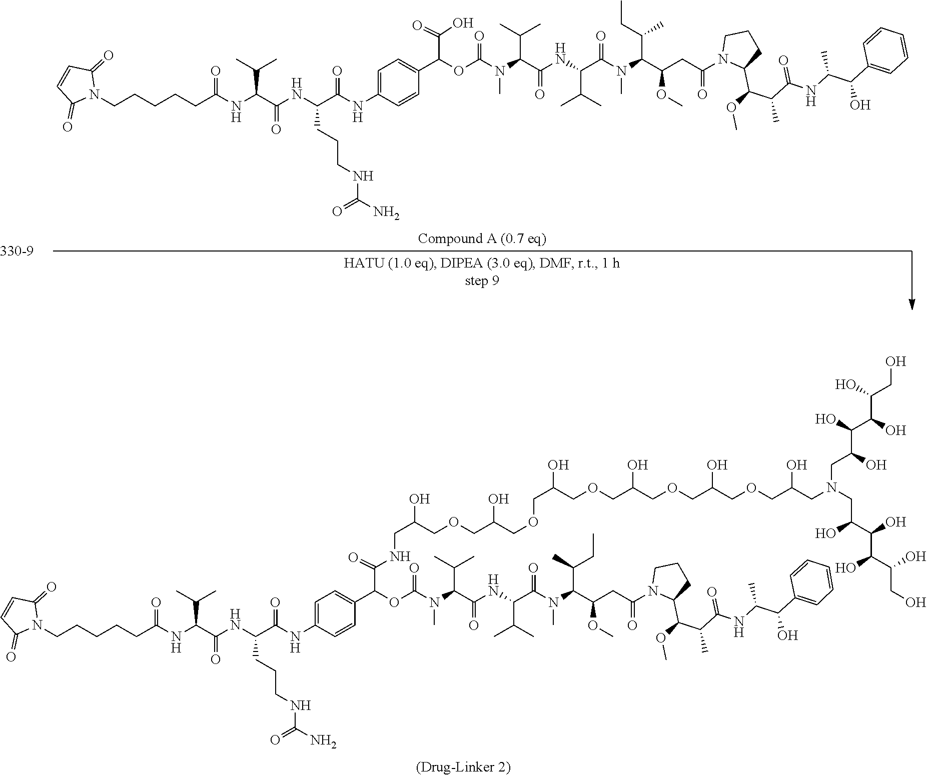

A solution of 330-9 (46 mg, 0.0558 mmol), Compound A (53 mg, 0.0390 mmol), HATU (21.2 mg, 0.0558 mmol) and DIPEA (21.6 mg, 0.167 mmol) in anhydrous DMF (4 mL) was stirred at room temperature for 1 h until Compound A was consumed. Then the reaction solution was purified by prep-HPLC to give Drug-Linker 2 (28 mg, 0.0131 mmol. 33.7%) as a white solid. LCMS, m/z=1066.11 (M/2+H)+. ¹H NMR (400 MHz, D₂O) δ 7.50-7.42 (m, 4H), 7.40-7.26 (m, 4H), 7.25-7.13 (m, 1H), 6.77 (s, 2H), 6.04-5.87 (m, 1H), 4.72-4.55 (m, 2H), 4.52-4.40 (m, 2H), 4.37-4.17 (m, 5H), 4.13-3.96 (m, 5H), 3.94-3.85 (m, 2H), 3.84-3.72 (m, 6H), 3.69-3.21 (m, 43H), 3.18-2.90 (m, 8H), 2.71 (s, 1H), 2.61-2.39 (m, 2H), 2.35-2.11 (m, 4H), 2.04 (d, J=9.8 Hz, 2H), 1.93-1.71 (m, 5H), 1.67-1.44 (m, 8H), 1.34-1.26 (m, 3H), 1.25-1.11 (m, 5H), 1.06 (d, J=6.4 Hz, 2H), 0.99-0.72 (m, 22H), 0.62-0.52 (m, 1H), 0.47-0.33 (m, 1H).

### Example 3: Preparation of Drug-Linker 3

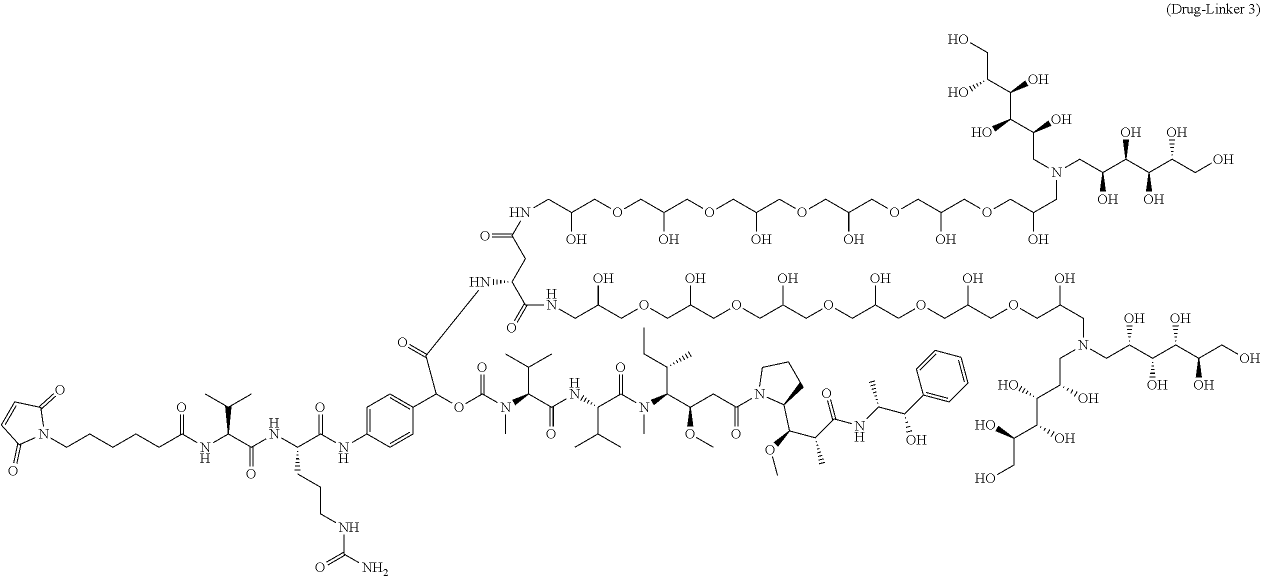 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@H](CC(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

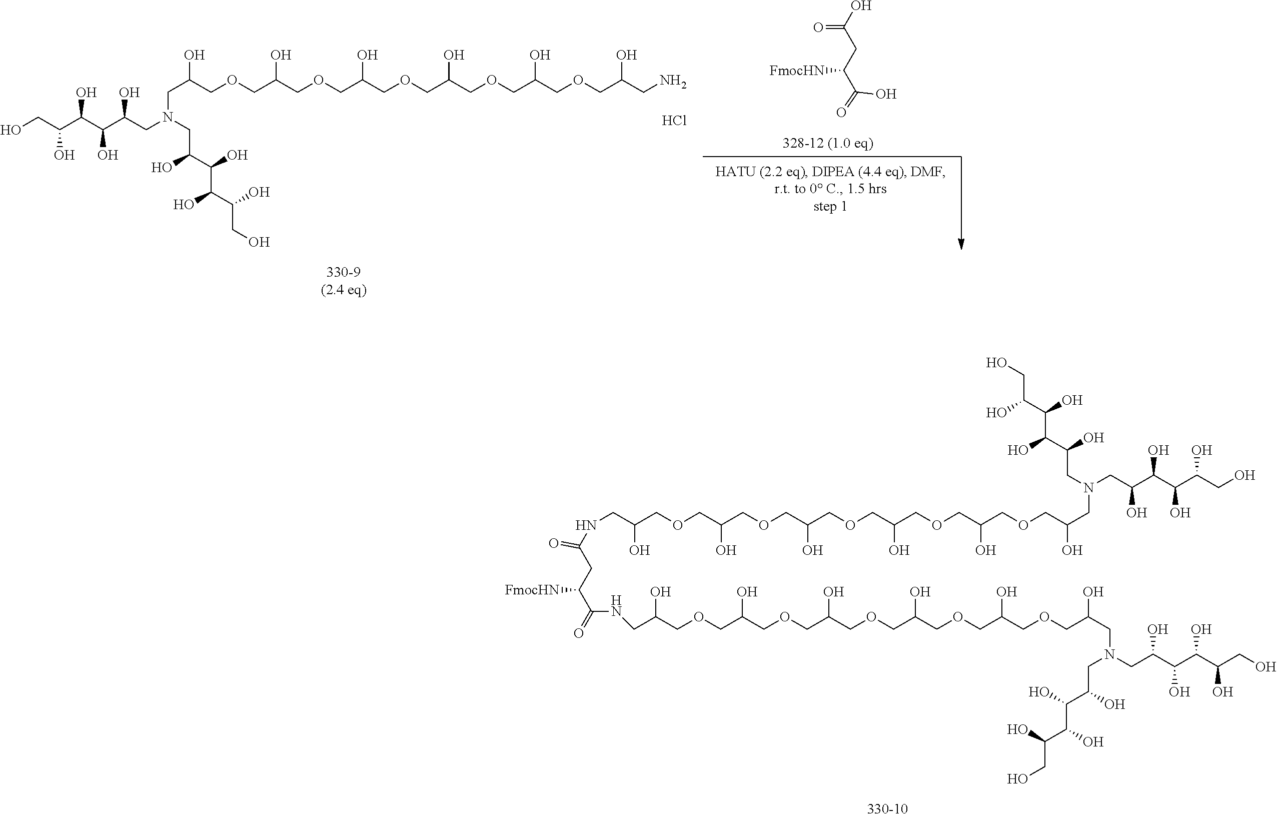

A solution of 328-12 (16.5 mg, 0.0465 mmol) and HATU (38.9 mg, 0.102 mmol) in anhydrous DMF (2 mL) was stirred at room temperature for 15 mins, then it was stirred in an ice bath. A solution of 330-9 (92 mg, 0.112 mmol) in anhydrous DMF (2 mL) was added dropwise, followed by DIPEA (26.5 mg, 0.205 mmol). The resulting mixture was stirred in the ice bath for 1 h until most of 328-12 was consumed. The reaction mixture was purified by reverse phase liquid chromatography to give 330-10 (14 mg, 0.00738 mmol, 15.9%) as a colorless oil.

### Step 2

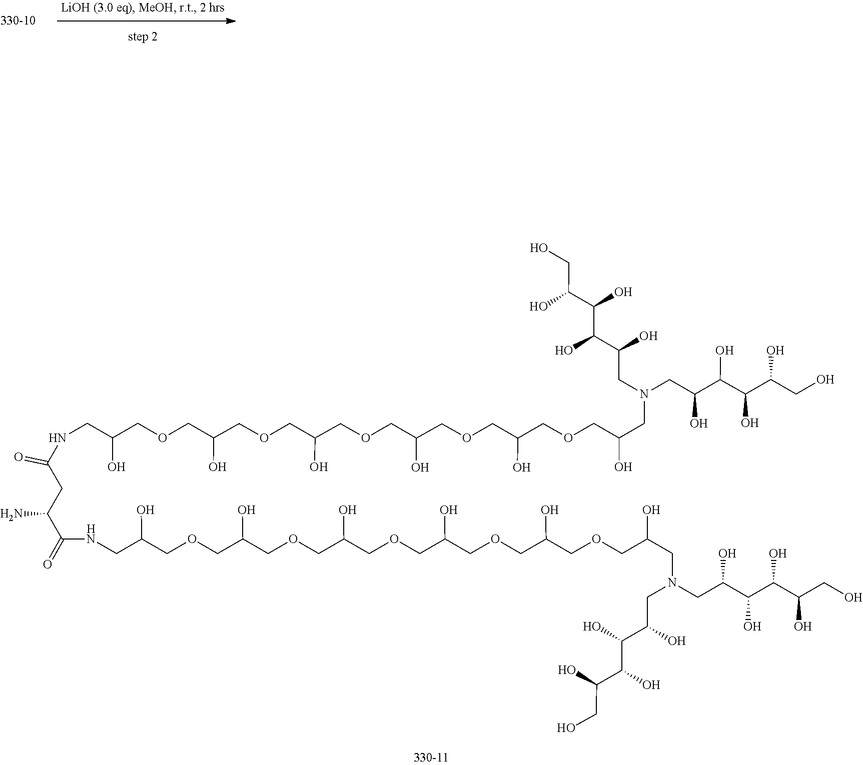 [SMILES: N[C@H](CC(=O)NCC(O)COCC(O)COCC(O)COCC(O)COCC(O)COCC(O)CN(C[C@H](O)C(O)[C@H](O)[C@H](O)CO)C[C@H](O)[C@@H](O)[C@H](O)[C@H](O)CO)C(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO]

To a solution of 330-10 (14 mg, 0.00738 mmol) in MeOH (2 mL) was added LiOH·H₂O (2 mg, 0.0442 mmol), and the mixture was stirred at room temperature for 2 hrs until 330-10 was consumed. The reaction solution was neutralized with 1N HCl to pH=7, and concentrated under reduced pressure to give a crude product, which was dissolved in H₂O (5 mL) and washed with hexane (2 mL×3). The aqueous phase was concentrated to dryness under reduced pressure to afford 330-11 (12.4 mg, 0.0074 mmol, 100%) as a white solid, which was used in the next step without further purification.

### Step 3

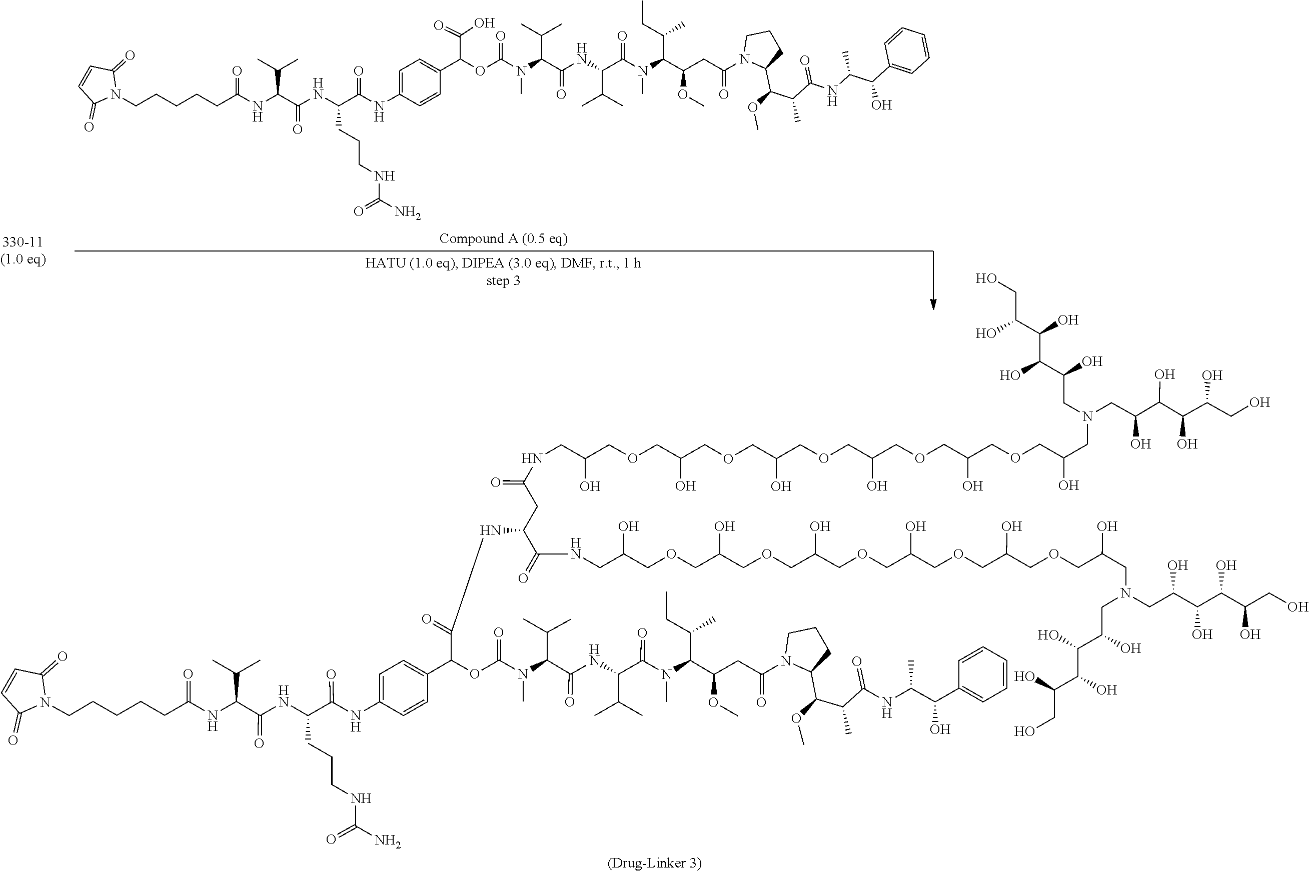

A solution of 330-11 (12 mg, 0.00716 mmol), Compound A (5.0 mg, 0.00368 mmol), HATU (2.7 mg, 0.0071 mmol) and DIPEA (2.8 mg, 0.0217 mmol) in anhydrous DMF (2 mL) was stirred at room temperature for 1 h until Compound A was consumed. Then the reaction solution was purified by prep-HPLC to give Drug-Linker 3 (5.0 mg, 0.00166 mmol, 45.1%) as a white solid. LCMS, m/z=1509.00 (M/2+H)+, m/z=1006.6 (M/3+H)+.

### Example 4: Preparation of Drug-Linker 4

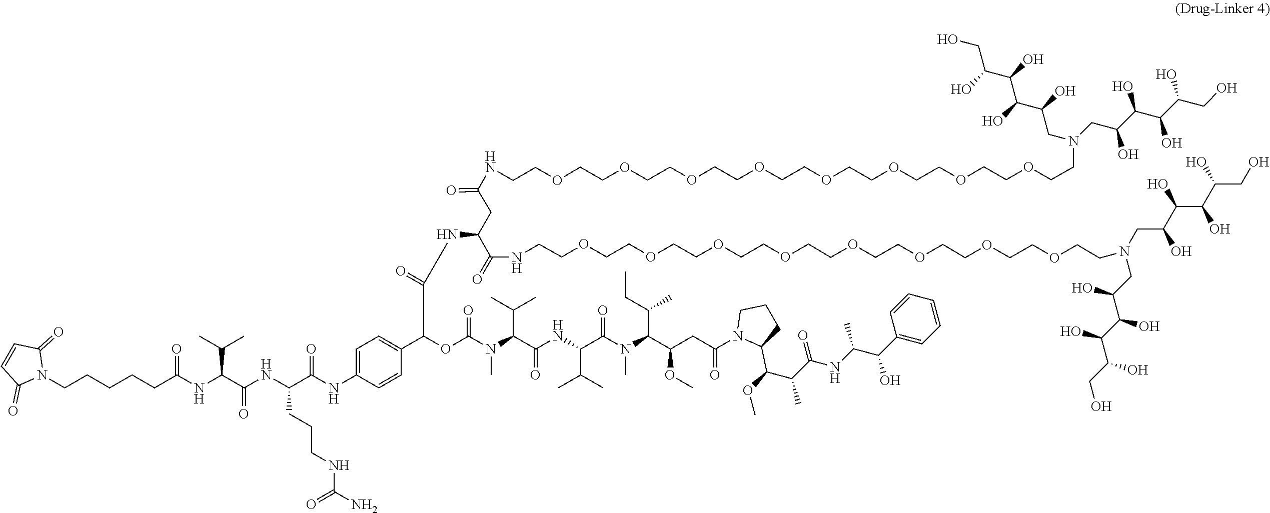 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@@H](CC(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

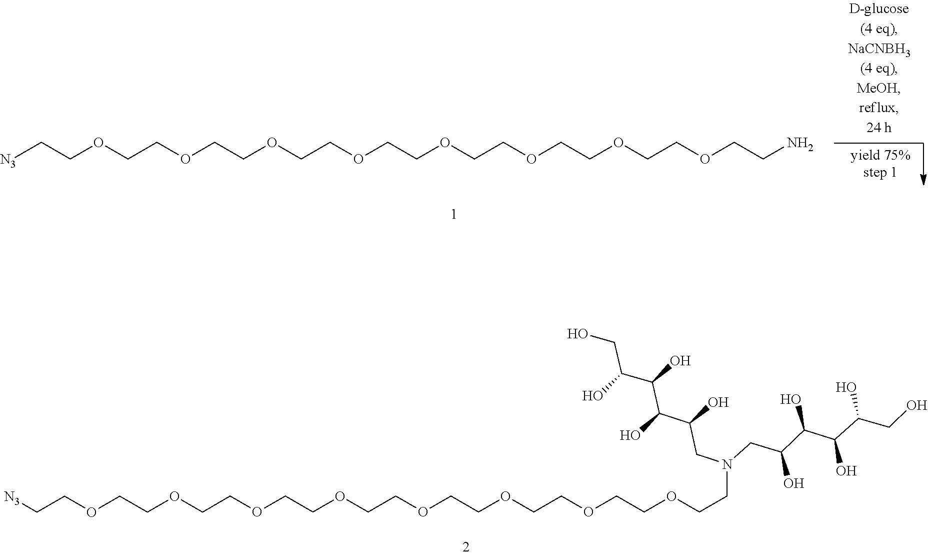 [SMILES: [N-]=[N+]=NCCOCCOCCOCCOCCOCCOCCOCCOCCN ; [N-]=[N+]=NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO]

To the solution of 1 (2.5 g, 5.701 mmol) in MeOH (20 mL) was added D-Glucose (4.11 g, 22.804 mmol) and NaBH₃CN (1.385 mL, 22.804 mmol). The mixture was stirred at reflux for 24h to complete. Then the resulting solution concentrated to dryness and the residue was purified by reverse phase chromatography (C8 column, eluting with 0-45% methanol in water with 0.01% TFA) to afford the product 2 as yellow oil. ESI m/z: 767.5 (M+H)⁺.

### Step 2

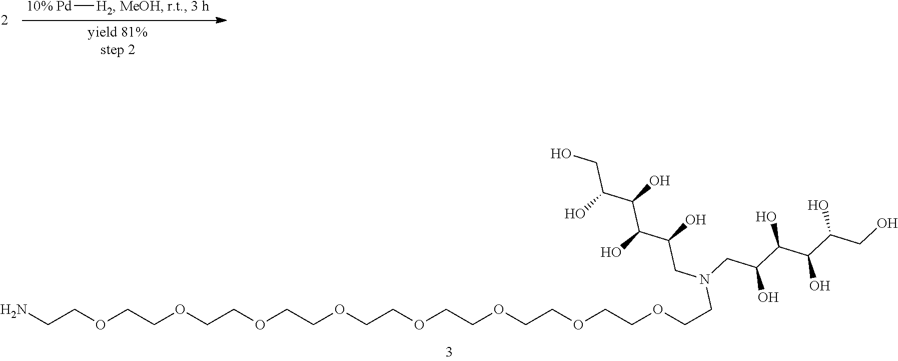

To the solution of 2 (3.3 g, 4.303 mmol) in MeOH (20 mL) was added Pd/C (10% wt, 330 mg) under nitrogen and equipped with H₂ balloon. The reaction system was degassed and backfilled with hydrogen for three times and then stirred at room temperature under hydrogen atmosphere for 3h to complete. The resulting mixture was filtered to remove catalyst solid and the filtrate was concentrated, then purified by reverse phase chromatography (C8 column, eluting with 0-25% acetonitrile in water with 0.01% TFA) to afford the product 3 (2.6 g, 3.510 mmol, 81.50%) as colorless oil. ESI m/z: 371.3 (M/2+H)+, 741.4 (M+H)⁺.

### Step 3

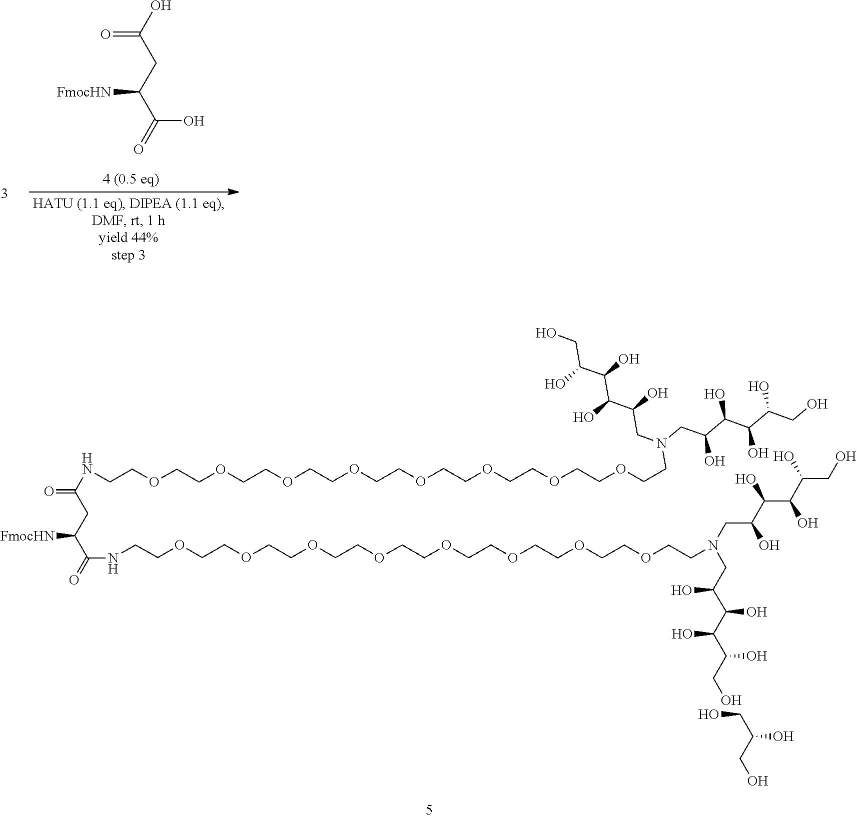

A solution of compound 5 (0.62 g, 1.755 mmol) in DMF (5 mL) was added HATU (1.47 g, 3.860 mmol) followed by DIPEA (0.50 g, 3.860 mmol). After stirring at room temperature for 15 min, the solution was added in a dropwise manner into the solution of 3 (2.6 g, 3.510 mmol) in DMF (5 mL). After addition, the solution was stirred at room temperature for another 1h to complete. The completed solution was then purified directly by reverse phase chromatography (C8 column, eluting with 0-40% acetonitrile in water with 0.01% TFA) to afford the product 5 (1.4 g, 0.777 mmol, 44.30%) as colorless oil. ESI m/z: 601.0 (M/3+H)⁺, 901.0 (M/2+H)⁺.

### Step 4

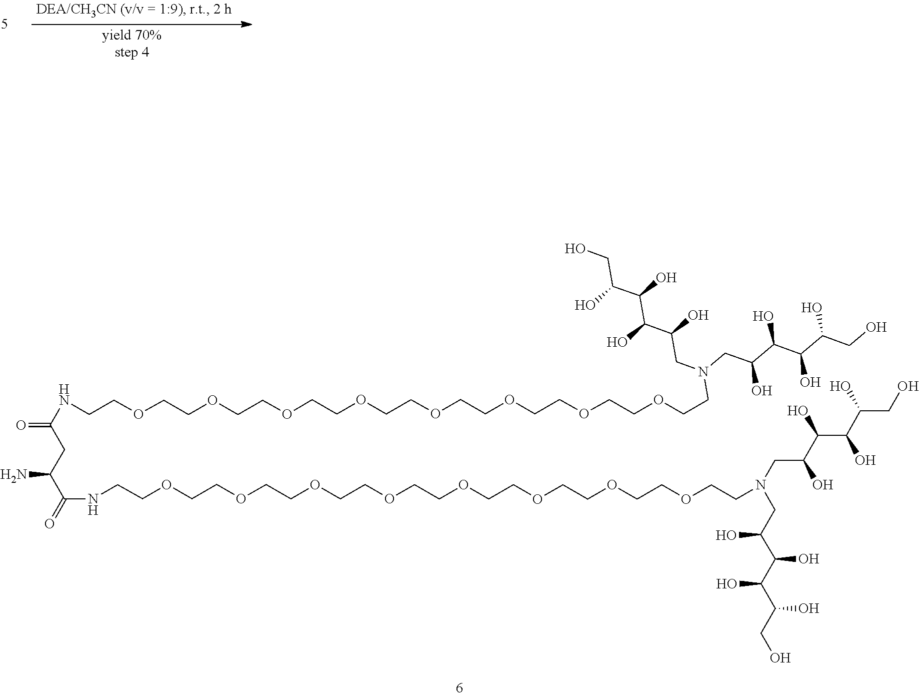

To the solution of 5 (1.4 g, 0.777 mmol) in MeCN (6 mL) was diethyl amine (0.7 mL, 8.930 mmol). The mixture was stirred at room temperature for 2h to achieve complete deprotection. Then the resulting solution was concentrated under reduced pressure to remove most of diethyl amine, and the residue was purified by reverse phase chromatography (C8 column, eluting with 0-20% acetonitrile in water with 0.01% TFA) to get desired fractions, which was freeze-dried to afford 6 as sticky colorless oil. ESI m/z: 526.9 (M/3+H)⁺, 789.9 (M/2+H)⁺. 1HNMR (400 MHz, DMSO-d6) δ 8.36 (t, J=5.6 Hz, 1H), 8.24 (t, J=5.6 Hz, 1H), 5.88-4.41 (m, 15H), 3.99-3.79 (m, 4H), 3.61-3.56 (m, 12H), 3.52-3.49 (m, 60H), 3.49-3.40 (m, 12H), 3.27-3.19 (m, 6H), 3.04-2.86 (m, 16H), 2.65-2.06 (m, 2H), 1.15 (t, J=7.2 Hz, 2H) ppm.

### Step 5

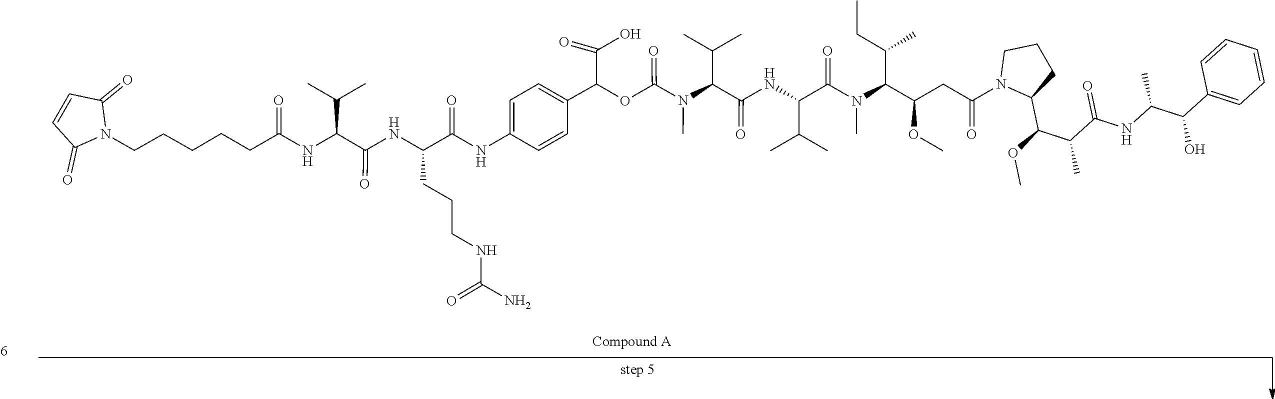 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)O)c1ccc(NC(=O)[C@H](CCCNC(N)=O)NC(=O)[C@@H](NC(=O)CCCCCN2C(=O)C=CC2=O)C(C)C)cc1)C(C)C]

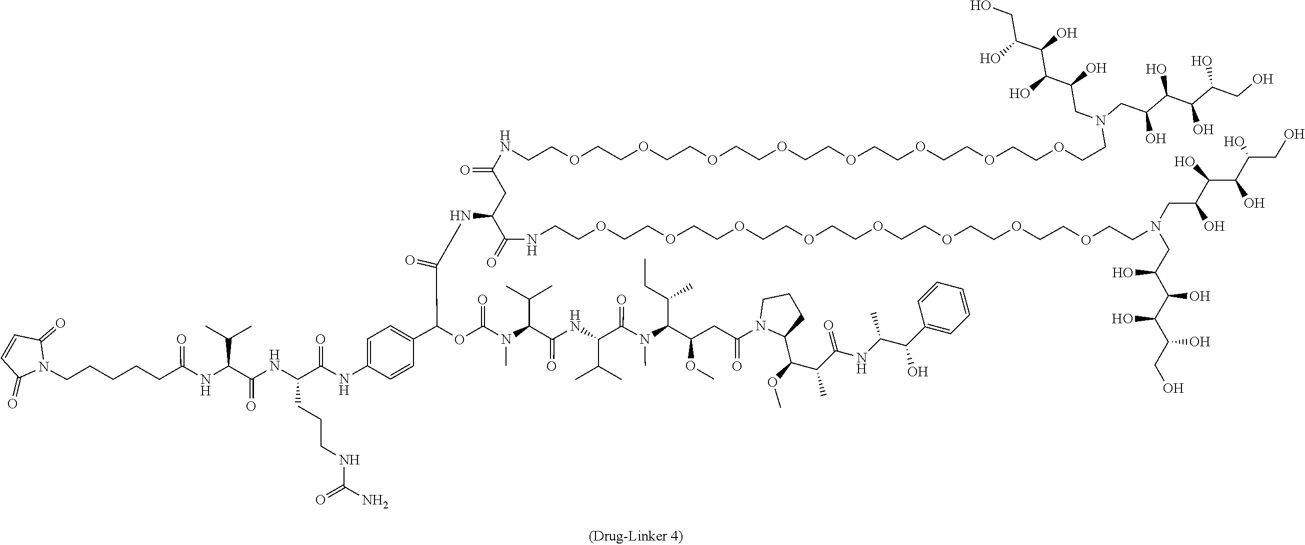

A solution of 6 (100 mg, 0.063 mmol), Compound A (86 mg, 0.063 mmol) and HATU (24 mg, 0.063 mmol) in anhydrous DMF (4 mL) was stirred at room temperature for 5 min, then DIPEA (25 mg, 0.193 mmol) was added. The resulting solution was stirred for another 1 hr at r.t. until LCMS indicated complete reaction. Then the reaction solution was purified by prep. HPLC to give Drug-Linker 4 (40 mg, 0.014 mmol, 21.73%) as a white solid. LCMS, m/z=1461.23 (M/2+H)⁺; ¹H NMR (400 MHz, D₂O) δ 7.54-7.48 (m, 2H), 7.45-7.37 (m, 2H), 7.37-7.26 (m, 5H), 6.73 (s, 2H), 4.40-4.38 (m, 2H), 4.19-4.15 (m, 6H), 3.85-3.83 (m, 5H), 3.78-3.74 (m, 10H), 3.71-3.66 (m, 13H), 3.63-3.61 (m, 49H), 3.58-3.56 (m, 17H), 3.49-3.43 (m, 14H), 3.39-3.35 (m, 4H), 3.30-3.29 (d, 3H), 3.24-3.23 (d, 4H), 3.06-3.00 (m, 4H), 2.77-2.64 (m, 2H), 2.22-2.21 (m, 2H), 1.99-1.97 (m, 2H), 1.75 (s, 5H), 1.53-1.45 (m, 10H), 1.24-1.08 (m, 9H), 1.02-1.01 (m, 2H), 0.92-0.75 (m, 28H) ppm.

### Example 5: Preparation of Drug-Linker 5

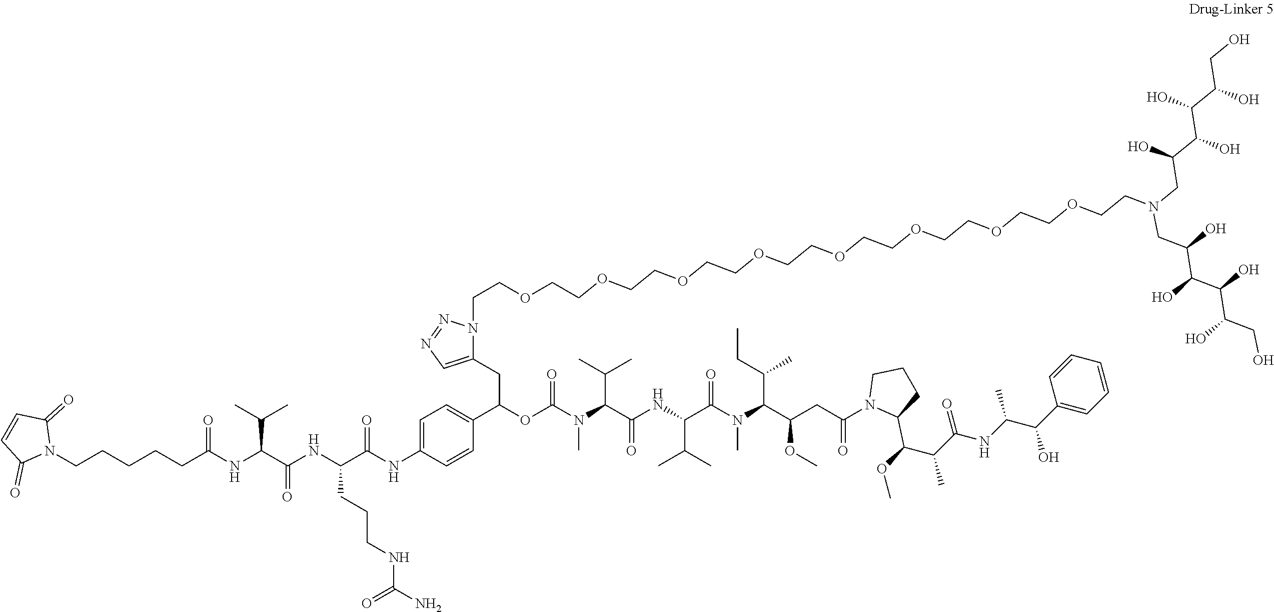 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(Cc1cnnn1CCOCCOCCOCCOCCOCCOCCOCCOCCN(C[C@@H](O)[C@H](O)[C@@H](O)[C@@H](O)CO)C[C@@H](O)[C@@H](O)[C@H](O)[C@@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

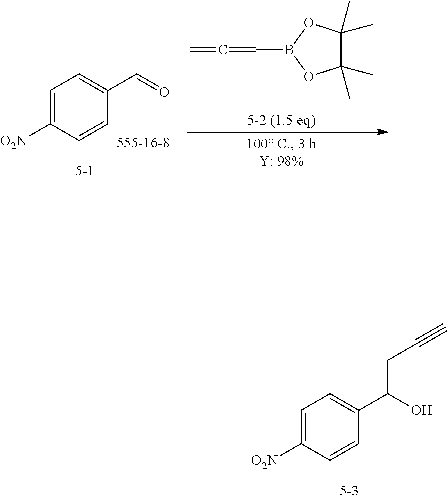

The mixture of 4-nitrobenzaldehyde 5-1 (10 g, 66.173 mmol) and 4,4,5,5-tetramethyl-2-(propa-1,2-dien-1-yl)-1,3,2-dioxaborolane 5-2 (17.532 mL, 99.259 mmol) was heated at 100° C. under nitrogen atmosphere with stirring for 3h to achieve complete conversion. Then the resulting solution was purified by flash chromatography (silica gel, eluting with 0-30% EA in PE) to afford the product 5-3 (12.6 g, 65.903 mmol, 99.60%) as a pale yellow solid. ESI m/z: 192.1 (M+H)⁺.

### Step 2

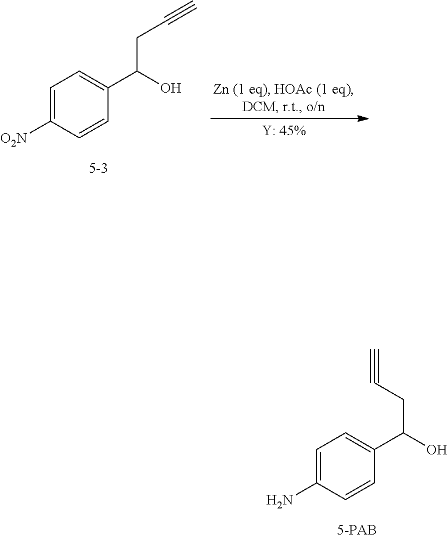

To the solution of 1-(4-nitrophenyl)but-3-yn-1-ol 5-3 (16 g, 83.686 mmol) in DCM (30 mL) was added Zn powder (0.767 mL, 83.686 mmol) and acetic acid (4.795 mL, 83.686 mmol). Then the mixture was stirred at room temperature for overnight. After completion, the mixture was filtered to remove the zinc solid, and the filtrate was concentrated and purified by reverse phase flash chromatography (C18 column, eluting with 0-15% acetonitrile in water with 0.01% TFA) to afford the product 1-(4-aminophenyl)but-3-yn-1-ol 5-PAB (6.1 g, 37.841 mmol, 45.22%) as a brown solid. ESI m/z: 162.2 (M+H)⁺.

### Step 3

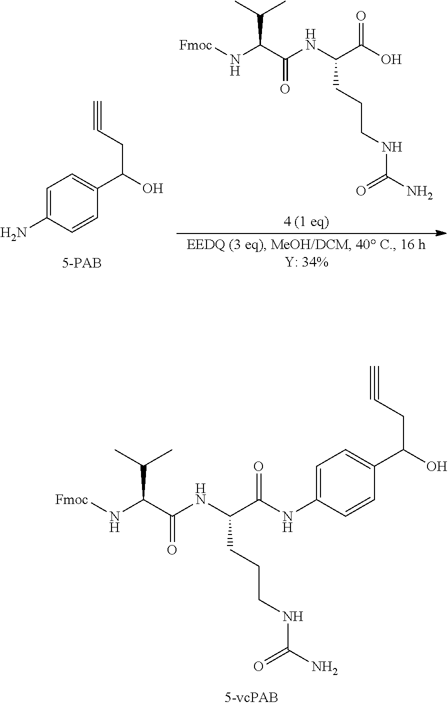

To the solution of (2S)-5-(carbamoylamino)-2-[(2S)-2-({[(9H-fluoren-9-yl)methoxy]carbonyl}amino)-3-methylbutanamido]pentanoic acid 4 (2.5 g, 5.035 mmol) in MeOH (8 mL) and DCM (32 mL) was added 1-(4-aminophenyl)but-3-yn-1-ol 5-PAB (0.81 g, 5.035 mmol) and EEDQ (3.74 g, 15.104 mmol). The mixture was stirred at 40° C. for overnight to complete. The resulting solution was concentrated to dryness and then purified by flash chromatography (silica gel, eluting with 0-10% methanol in DCM) to afford the product (9H-fluoren-9-yl)methyl N-[(1S)-1-{[(1S)-4-(carbamoylamino)-1-{[4-(1-hydroxybut-3-yn-1-yl)phenyl]carbamoyl}butyl]carbamoyl}-2-methylpropyl]carbamate 5-vcPAB (1.04 g, 1.626 mmol, 32.30%) as a pale yellow solid. ESI m/z: 640.3 (M+H)⁺. ¹H NMR (400 MHz, DMSO-d6) δ 9.99 (s, 1H), 8.10 (d, J=7.6 Hz, 1H), 7.88 (d, J=7.6 Hz, 2H), 7.76-7.72 (m, 2H), 7.53 (d, J=8.8 Hz, 2H), 7.45-7.39 (m, 3H), 7.32-7.26 (m, 3H), 5.96 (m, 1H), 5.44-5.39 (m, 3H), 4.62 (t, J=6.0 Hz, 1H), 4.42-4.40 (m, 1H), 4.31-4.20 (m, 3H), 3.95-3.90 (m, 1H), 3.00-2.91 (m, 2H), 2.70-2.69 (m, 1H), 2.45-2.32 (m, 2H), 2.00-1.97 (m, 1H), 1.59-1.23 (m, 5H), 0.88-0.83 (m, 6H) ppm.

### Step 4

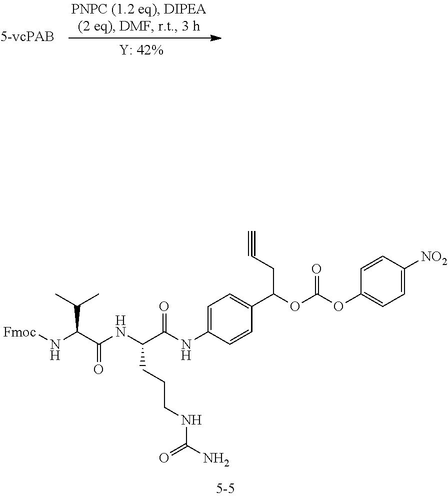

To the solution of (9H-fluoren-9-yl)methyl N-[(1S)-1-{[(1S)-4-(carbamoylamino)-1-{[4-(1-hydroxybut-3-yn-1-yl)phenyl]carbamoyl}butyl]carbamoyl}-2-methylpropyl]carbamate 5-vcPAB (1.04 g, 1.626 mmol) and DIPEA (0.42 g, 3.252 mmol) in DMF (15 mL) was added bis(4-nitrophenyl) carbonate (PNPC, 0.59 g, 1.951 mmol) portion wise. After addition, the mixture was stirred at room temperature for another 3 h. After completion according to LCMS monitoring, the resulting solution was purified directly by reverse phase flash chromatography (C18 column, eluting with 0-75% acetonitrile in water with 0.01% TFA) to afford the product 1-{4-[(2S)-5-(carbamoylamino)-2-[(2S)-2-({[(9H-fluoren-9-yl)methoxy]carbonyl}amino)-3-methyl butanamido]pentanamido]phenyl}but-3-yn-1-yl 4-nitrophenyl carbonate 5-5 (560 mg, 0.696 mmol, 42.75%) as a pink solid. ESI m/z: 805.3 (M+H)⁺.

### Step 5

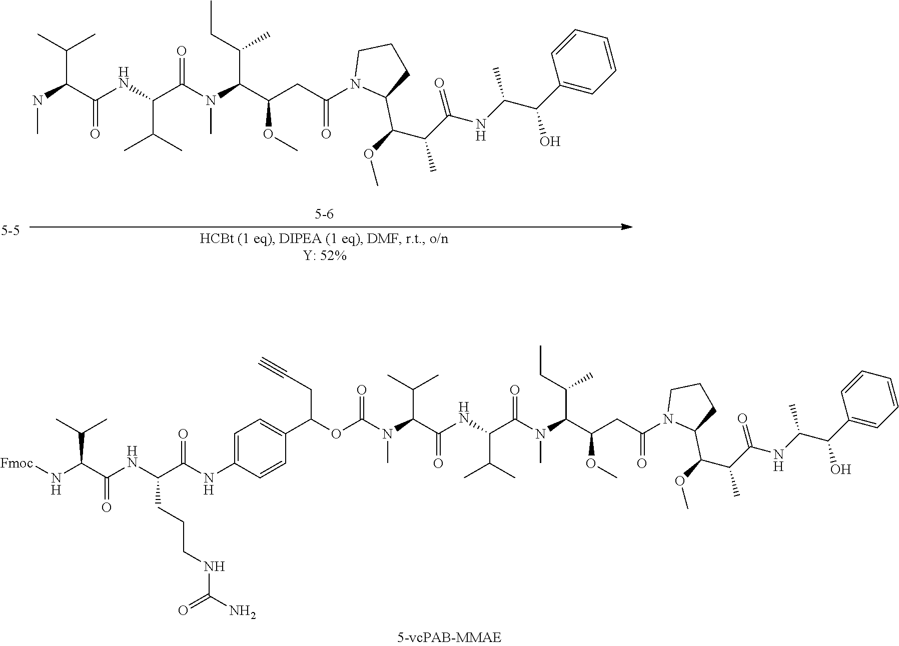

To the solution of 1-{4-[(2S)-5-(carbamoylamino)-2-[(2S)-2-({[(9H-fluoren-9-yl)methoxy]carbonyl}amino)-3-methylbutanamido]pentanamido]phenyl}but-3-yn-1-yl 4-nitrophenyl carbonate 5-5 (540 mg, 0.671 mmol) in DMF (15 mL) was added (2S)—N-[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]-3-methyl-2-(methylamino)butanamide 5-6 (481.72 mg, 0.671 mmol), HOBt (90.66 mg, 0.671 mmol) and DIPEA (86.71 mg, 0.671 mmol). The mixture was stirred at room temperature for overnight to achieve a complete reaction. Then the resulting solution was purified directly by reverse phase flash chromatography (C8 column, eluting with 0-70% acetonitrile in water with 0.01% TFA) to afford the product 1-{4-[(2S)-5-(carbamoylamino)-2-[(2S)-2-({[(9H-fluoren-9-yl)methoxy]carbonyl}amino)-3-methylbutanamido]pentanamido]phenyl}but-3-yn-1-yl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate 5-vcPAB-MMAE (486 mg, 0.351 mmol, 52.34%) as a white solid. ESI m/z: 692.3 (M/2+H)⁺.

### Step 6

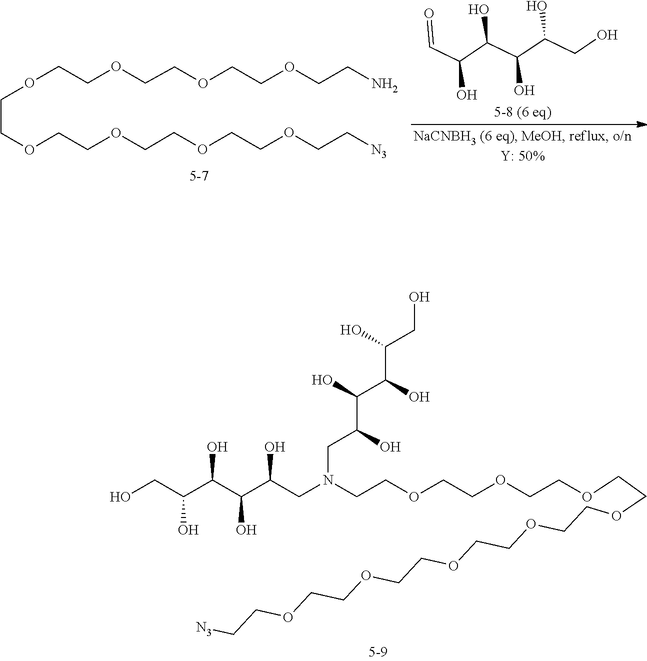 [SMILES: [N-]=[N+]=NCCOCCOCCOCCOCCOCCOCCOCCOCCN ; O=C[C@H](O)[C@@H](O)[C@H](O)[C@H](O)CO ; [N-]=[N+]=NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO]

A solution of 26-azido-3,6,9,12,15,18,21,24-octaoxahexacosan-1-amine 5-7 (160 mg, 0.365 mmol) and D-glucose 5-8 (394.40 mg, 2.189 mmol) in MeOH (6 mL) was treated with NaBH₃CN (0.133 mL, 2.189 mmol). Then the mixture was stirred at 75° C. for overnight. Then the complete reaction solution was concentrated to dryness and the residue was purified by reverse phase flash chromatography (C8 column, eluting with 0-10% acetonitrile in water with 0.01% TFA) to afford the product (29S,30R,31R,32R)-1-azido-27-((2S,3R,4R,5R)-2,3,4,5,6-pentahydroxyhexyl)-3,6,9,12,15,18,21,24-octaoxa-27-azatritriacontane-29,30,31,32,33-pentaol 5-9 (140 mg, 50%) as transparent oil. ESI m/z: 767.3 (M+H)⁺.

Step 7

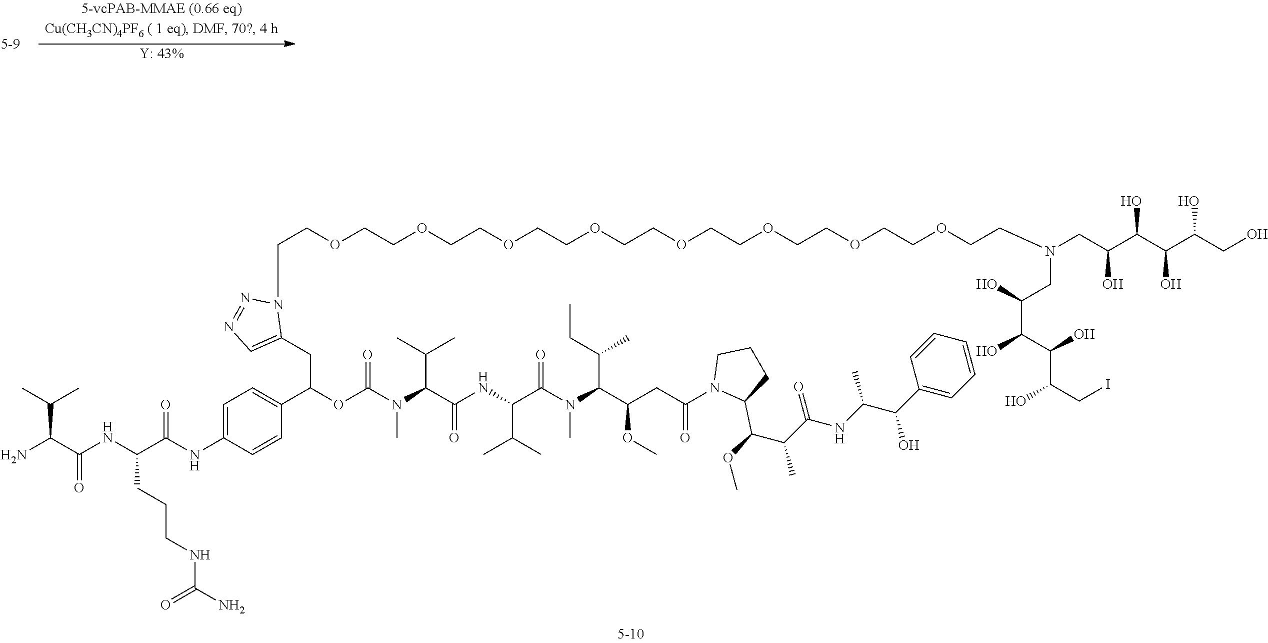 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(Cc1cnnn1CCOCCOCCOCCOCCOCCOCCOCCOCCN(C[C@H](O)[C@H](O)[C@@H](O)[C@@H](O)CI)C[C@H](O)[C@@H](O)[C@H](O)[C@H](O)CO)c1ccc(NC(=O)[C@H](CCCNC(N)=O)NC(=O)[C@@H](N)C(C)C)cc1)C(C)C]

To the solution of 1-{4-[(2S)-5-(carbamoylamino)-2-[(2S)-2-({[(9H-fluoren-9-yl)methoxy]carbonyl}amino)-3-methylbutanamido]pentanamido]phenyl}but-3-yn-1-yl N-[(1S)-1-{[(1S)-1—{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate 5-vcPAB-MMAE (300 mg, 0.217 mmol) in DMF (6 mL) was added (29S,30R,31R,32R)-1-azido-27-((2S,3R,4R,5R)-2,3,4,5,6-pentahydroxyhexyl)-3,6,9,12,15,18,21,24-octaoxa-27-azatritriacontane-29,30,31,32,33-pentaol 5-9 (249.38 mg, 0.325 mmol) and Cu(CH₃CN)₄PF₆ (121.55 mg, 0.325 mmol) at room temperature. The resulting mixture was heated at 70° C. with stirring for 4 h. The completed reaction solution was then purified by reverse phase flash chromatography (C8 column, eluting with 0-37% acetonitrile in water with 0.01% TFA) to afford the product 1-{4-[(2S)-5-(carbamoylamino)-2-[(2S)-2-({[(9H-fluoren-9-yl)methoxy]carbonyl}amino)-3-methylbutanamido]pentanamido]phenyl}-2-{1-[(29S,30R,31R,32R)-29,30,31,32,33-pentahydroxy-27-[(2S,3R,4R,5R)-2,3,4,5,6-pentahydroxyhexyl]-3,6,9,12,15,18,21,24-octaoxa-27-azatritriacontan-1-yl]-1H-1,2,3-triazol-5-yl}ethyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate 5-10 (204 mg, 0.095 mmol, 43.71%) as a white solid. ESI m/z: 1075.6 (M/2+H)⁺.

### Step 8

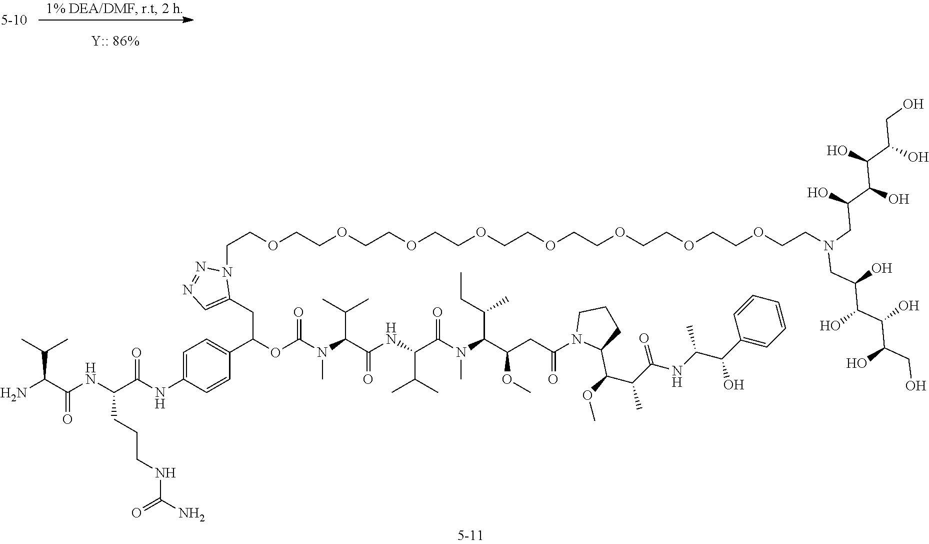 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(Cc1cnnn1CCOCCOCCOCCOCCOCCOCCOCCOCCN(C[C@@H](O)[C@H](O)[C@@H](O)[C@@H](O)CO)C[C@@H](O)[C@@H](O)[C@H](O)[C@H](O)CO)c1ccc(NC(=O)[C@H](CCCNC(N)=O)NC(=O)[C@@H](N)C(C)C)cc1)C(C)C]

To the solution of 1-{4-[(2S)-5-(carbamoylamino)-2-[(2S)-2-({[(9H-fluoren-9-yl)methoxy]carbonyl}amino)-3-methylbutanamido]pentanamido]phenyl}-2-{1-[(29S,30R,31R,32R)-29,30,31,32,33-pentahydroxy-27-[(2S,3R,4R,5R)-2,3,4,5,6-pentahydroxyhexyl]-3,6,9,12,15,18,21,24-octaoxa-27-azatritriacontan-1-yl]-1H-1,2,3-triazol-5-yl}ethyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate 5-10 (167 mg, 0.078 mmol) in DMF (2 mL) was added diethylamine (DEA, 0.02 mL, 0.125 mmol). The mixture was stirred at room temperature for 2h to complete. The resulting solution was purified by reverse phase flash chromatography (C8 column, eluting with 0-40% acetonitrile in water with 0.01% TFA) to afford the product 1-{4-[(2S)-2-[(2S)-2-amino-3-methylbutanamido]-5-(carbamoylamino)pentanamido]phenyl}-2-{1-[(29S,30R,31R,32R)-29,30,31,32,33-pentahydroxy-27-[(2S,3R,4R,5R)-2,3,4,5,6-pentahydroxyhexyl]-3,6,9,12,15,18,21,24-octaoxa-27-azatritriacontan-1-yl]-1H-1,2,3-triazol-5-yl}ethyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate 5-11 (130 mg, 0.067 mmol, 86.81%) as a white solid. ESI m/z: 965.5 (M/2+H)⁺.

### Step 9

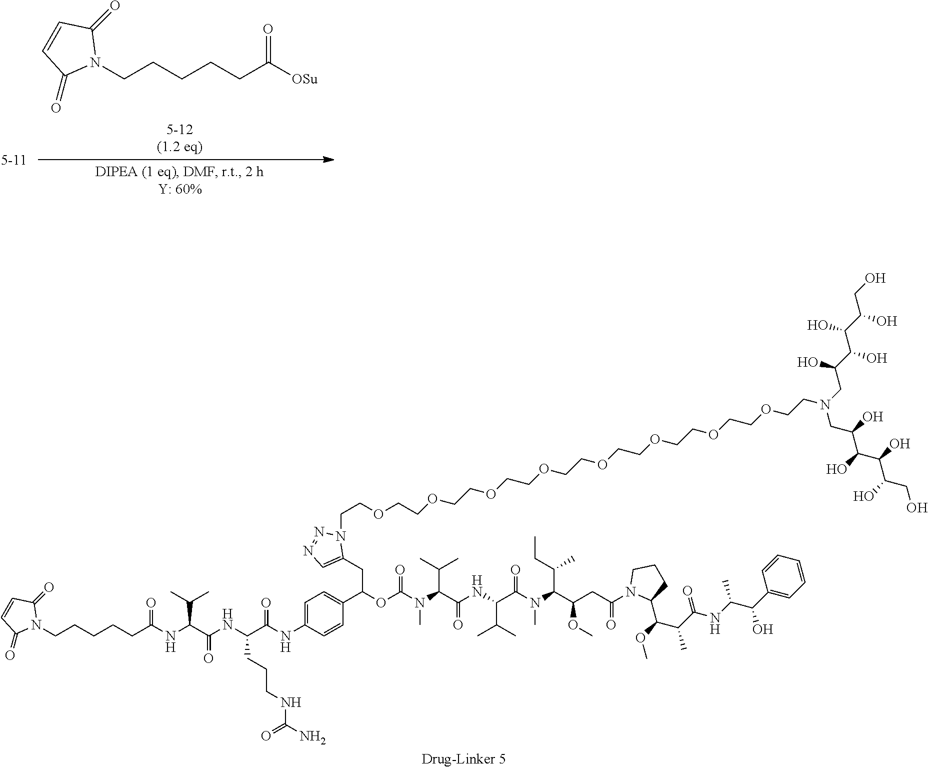

To the solution of 1-{4-[(2S)-2-[(2S)-2-amino-3-methylbutanamido]-5-(carbamoylamino)pentanamido]phenyl}-2-{1-[(29S,30R,31R,32R)-29,30,31,32,33-pentahydroxy-27-[(2S,3R,4R,5R)-2,3,4,5,6-pentahydroxyhexyl]-3,6,9,12,15,18,21,24-octaoxa-27-azatritriacontan-1-yl]-1H-1,2,3-triazol-5-yl}ethyl N-[(1S)-1-{[(1S)-1-{[(3S,4S,5S)-1-[(2S)-2-[(1R,2R)-2-{[(1R,2S)-1- hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate 5-11 (130 mg, 0.067 mmol) in DMF (3 mL) was added 2,5-dioxopyrrolidin-1-yl 6-(2,5-dioxo-2,5-dihydro-1H-pyrrol-1-yl)hexanoate 5-12(24.79 mg, 0.080 mmol) and DIPEA (8.66 mg, 0.067 mmol) sequentially. The resulting solution was stirred at room temperature for 2h to complete full conversion. The reaction solution was then purified directly by reverse phase flash chromatography (C8 column, eluting with 0-40% acetonitrile in water with 001% TFA) to afford the product 1-{4-[(2S)-5-(carbamoylamino)-2-[(2S)-2-[6-(2,5-dioxo-2,5-dihydro-1H-pyrrol-1-yl)hexanamido]-3-methylbutanamido]pentanamido]phenyl}-2-{1-[(29S,30R,31R,32R)-29,30,31,32,33-pentahydroxy-27-[(2S,3R,4R,5R)-2,3,4,5,6-pentahydroxyhexyl]-3,6,9,12,15,18,21,24-octaoxa-27-azatritriacontan-1-yl]-1H-1,2,3-triazol-5-yl}ethyl N-[(1S)-1-{[(1S)-1-{[(3S,4S,5S)-1-[(2S)-2-[(1R,2R)-2-{[(1R,2S)-1- hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (86 mg, 0.041 mmol, 60.13%) Drug-Linker 5 as a white solid. ESI m/z: 1061.8 (M/2+H)⁺, retention time 6.062 min (HPLC). ¹H NMR (400 MHz, DMSO-d6) δ 9.95 (s, 1H), 8.60-8.45 (m, 1H), 8.20-8.06 (m, 2H), 7.95-7.90 (m, 1H), 7.83-7.80 (m, 1H), 7.72-7.60 (m, 1.5H), 7.54-7.50 (m, 2H), 7.43-7.40 (m, 1H), 7.31-7.12 (m, 6H), 6.99 (s, 2H), 6.03 (brs, 1H), 5.82-5.68 (m, 1.5H), 5.50-5.30 (m, 3H), 4.76-4.52 (m, 3H), 4.50-4.30 (m, 8H), 4.29-4.11 (m, 3H), 4.00-3.92 (m, 8H), 3.77-3.66 (m, 16H), 3.66-3.48 (m, 18H), 3.45-3.29 (m, 12H), 3.24-3.07 (m, 12H), 3.00-2.68 (m, 8H), 2.44-1.94 (m, 7H), 1.80-1.67 (m, 4H), 1.58-1.34 (m, 10H), 1.21-1.15 (m, 2H), 1.05-0.96 (m, 7H), 0.90-0.50 (m, 28H) ppm.

### Example 6: Preparation of Drug-Linker 6

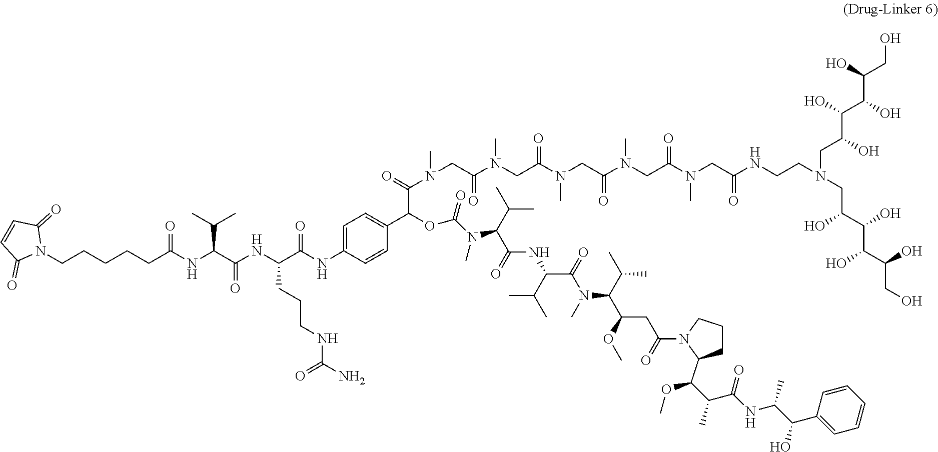

### Step 1

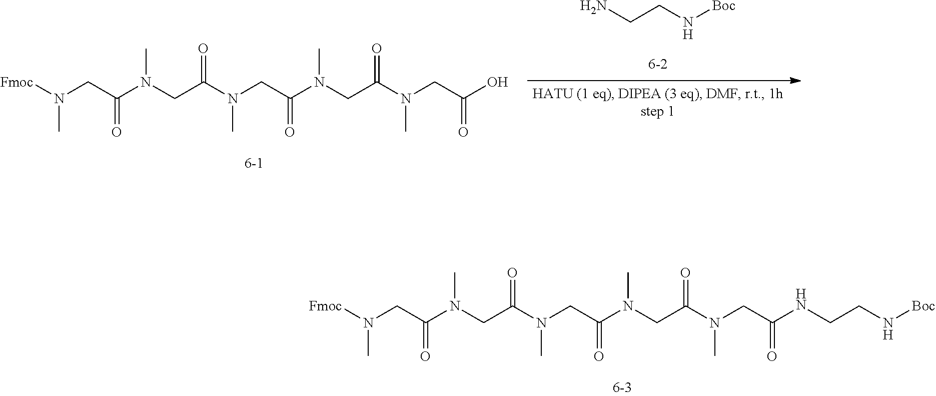

A solution of 6-1 (300 mg, 0.504 mmol), 6-2 (81 mg, 0.504 mmol) and HATU (192 mg, 0.504 mmol) in anhydrous DMF (10 mL) was stirred at room temperature for 5 min, then DIPEA (196 mg, 1.512 mmol) was added. The resulting solution was stirred for another 1 hr at r.t. until LCMS indicated a complete reaction. The reaction solution was purified directly by reverse phase liquid chromatography to give 6-3 (227 mg, 0.308 mmol, 61.19%) as a white solid.

### Step 2

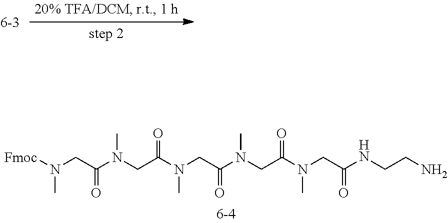

A solution of 6-3 (227 mg, 0.308 mmol) and TFA (2 mL) in anhydrous DCM (8 mL) was stirred at room temperature for 1 hr until LCMS indicated all starting amine was disappeared and desired product was detected. Then the solution was concentrated to dryness to 6-4 (185 mg, 0.290 mmol, 94.18%) as yellow oil, used as such in the next step.

### Step 3

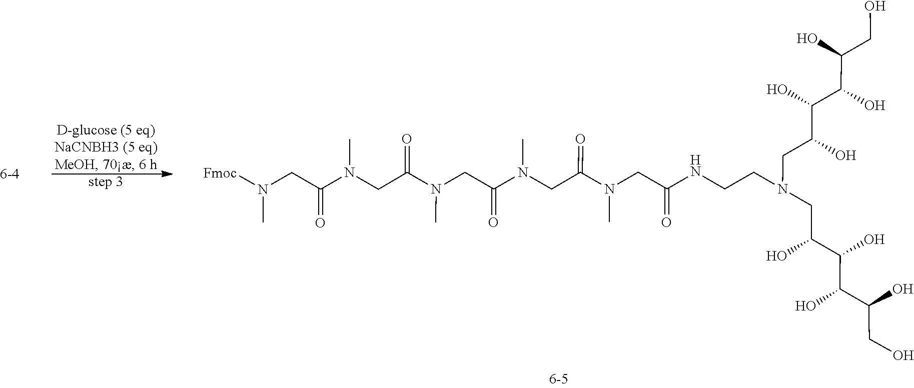

A solution of 6-4 (185 mg, 0.290 mmol) and D-Glucose (261 mg, 1.450 mmol) in anhydrous Methanol (50 mL) was heated at 50° C. for 30 min, then NaCNBH₃ (92 mg, 1.450 mmol) was added. The resulting solution was stirred for another 6 hr at 70° C. until indicated all starting amine was disappeared and the mass of desired product was detected. Then the reaction solution was concentrated and purified by reverse phase liquid chromatography to give 6-5 (125 mg, 0.129 mmol, 44.48%) as a white solid.

### Step 4

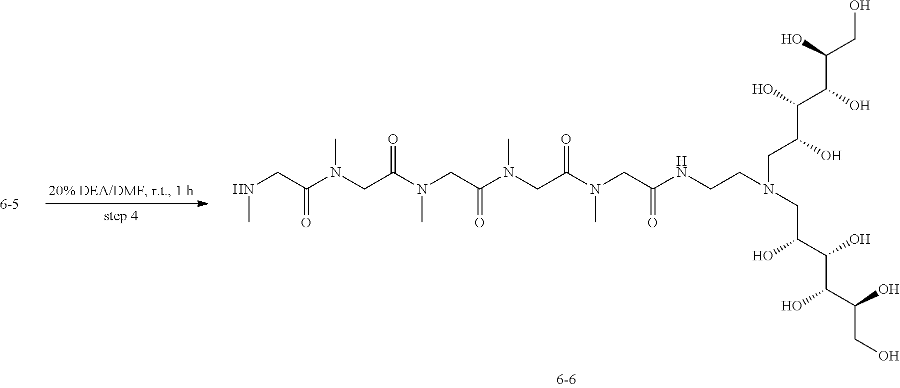 [SMILES: CNCC(=O)N(C)CC(=O)N(C)CC(=O)N(C)CC(=O)N(C)CC(=O)NCCN(C[C@@H](O)[C@H](O)[C@@H](O)[C@@H](O)CO)C[C@@H](O)[C@H](O)[C@@H](O)[C@@H](O)CO]

A solution of 6-5 (125 mg, 0.129 mmol) and DEA (1 mL) in anhydrous DMF (4 mL) was stirred at room temperature for 1 hr until LCMS indicated all starting amine was disappeared and desired product was detected. Then the solution was concentrated to dryness to 6-6 (91 mg, 0.122 mmol, 94.57%) as colorless oil, used as such in the next step.

### Step 5

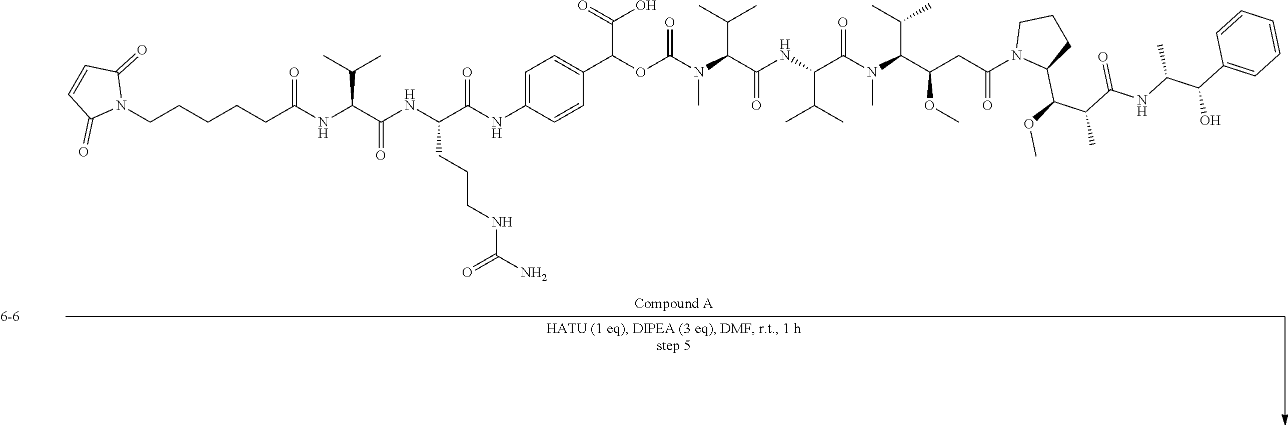

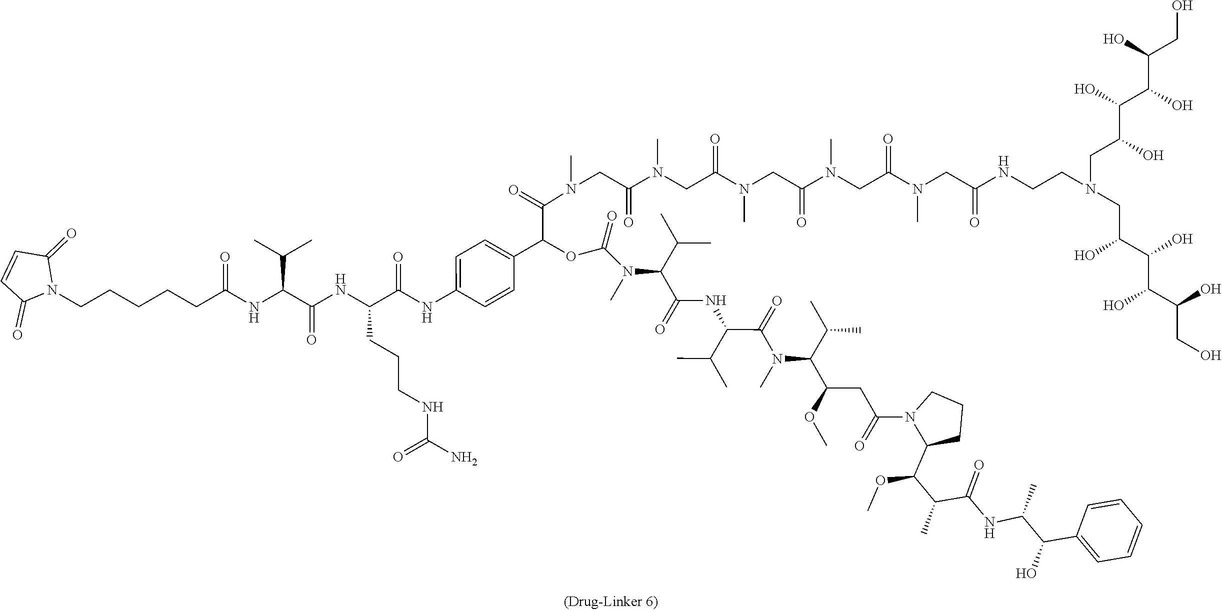

A solution of 6-6 (12 mg, 0.015 mmol), Compound A (20 mg, 0.015 mmol) and HATU (6 mg, 0.015 mmol) in anhydrous DMF (4 mL) was stirred at room temperature for 5 min, then DIPEA (6 mg, 0.045 mmol) was added. The resulting solution was stirred for another 1 hr at r.t. until LCMS indicated complete reaction. Then the reaction solution was purified by prep. HPLC to give Drug-Linker 6 (12 mg, 0.006 mmol, 38.35%) as a white solid. LCMS, m/z=1044.15 (M/2+H)⁺; ¹H NMR (400 MHz, D₂O) δ 7.46-7.44 (m, 4H), 7.27-7.21 (m, 7H), 4.35-4.26 (m, 9H), 4.14-3.96 (m, 10H), 3.79-3.62 (m, 10H), 7.00 (s, 2H), 3.36-3.10 (m, 16H), 3.05-2.79 (m, 28H), 2.67-2.55 (m, 7H), 2.42-2.17 (m, 5H), 1.80-1.66 (m, 5H), 1.53-1.40 (m, 10H), 1.2-0.63 (m, 37H).

### Example 7: Preparation of Drug-Linker 7

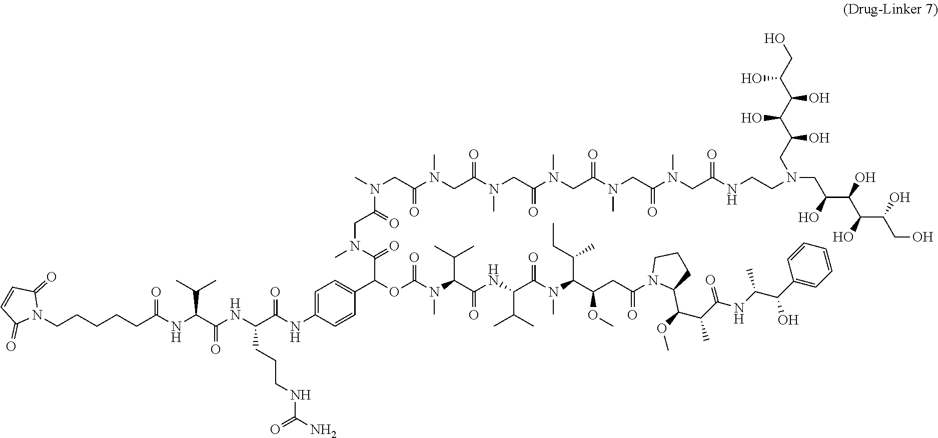 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)CC(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

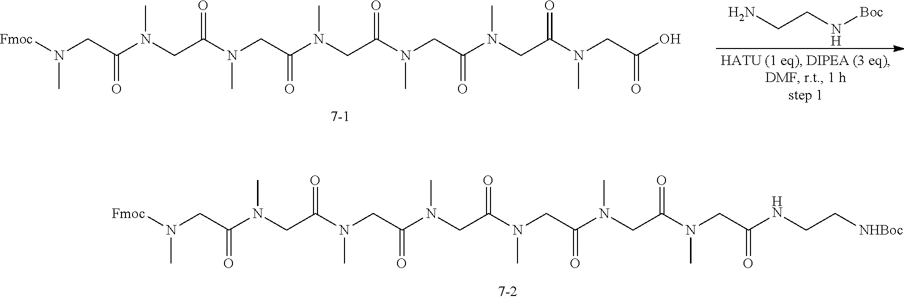

A solution of 7-1 (300 mg, 0.407 mmol), tert-butyl (2-aminoethyl)carbamate (65 mg, 0.407 mmol) and HATU (155 mg, 0.407 mmol) in anhydrous DMF (10 mL) was stirred at room temperature for 5 min, then DIPEA (158 mg, 1.221 mmol) was added. The resulting solution was stirred for another 1 hr at r.t. until LCMS indicated complete reaction. The reaction solution was purified directly by reverse phase liquid chromatography to give 7-2 (243 mg, 0.276 mmol, 67.81%) as a white solid

### Step 2

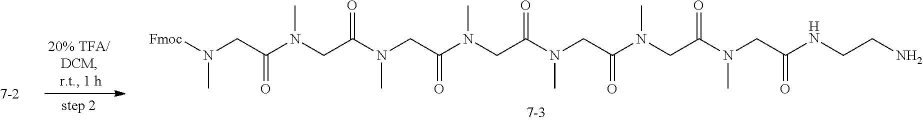

A solution of 7-2 (243 mg, 0.276 mmol) and TFA (2 mL) in anhydrous DCM (8 mL) was stirred at room temperature for 1 hr until LCMS indicated all starting amine was disappeared and desired product was detected. Then the solution was concentrated to dryness to 7-3 (207 mg, 0.265 mmol, 96.17%) as yellow oil, used as such in the next step.

### Step 3

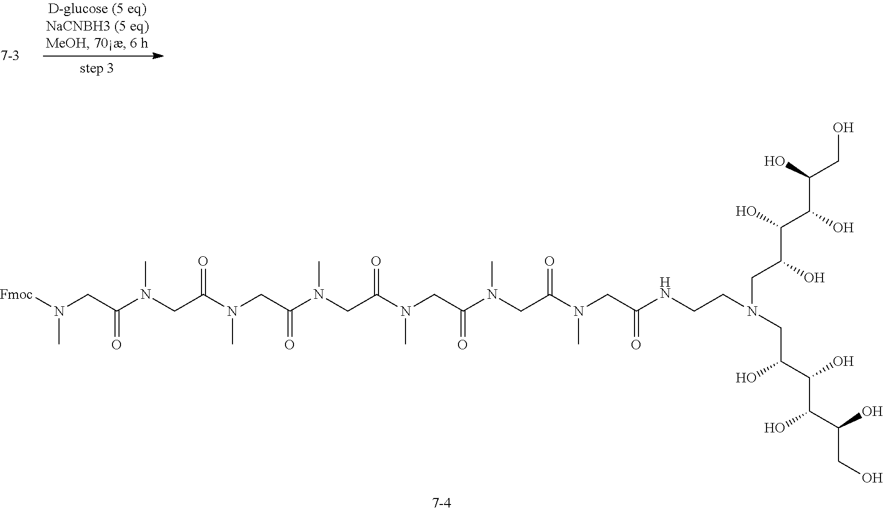

A solution of 7-3 (207 mg, 0.265 mmol) and D-Glucose (239 mg, 1.325 mmol) in anhydrous Methanol (50 mL) was heated at 50° C. for 30 min, then NaCNBH₃ (83 mg, 1.325 mmol) was added. The resulting solution was stirred for another 6 hr at 70° C. until indicated all starting amine was disappeared and the mass of desired product was detected. Then the reaction solution was concentrated and purified by reverse phase liquid chromatography to give 7-4 (147 mg, 0.133 mmol, 50.19%) as a white solid.

### Step 4

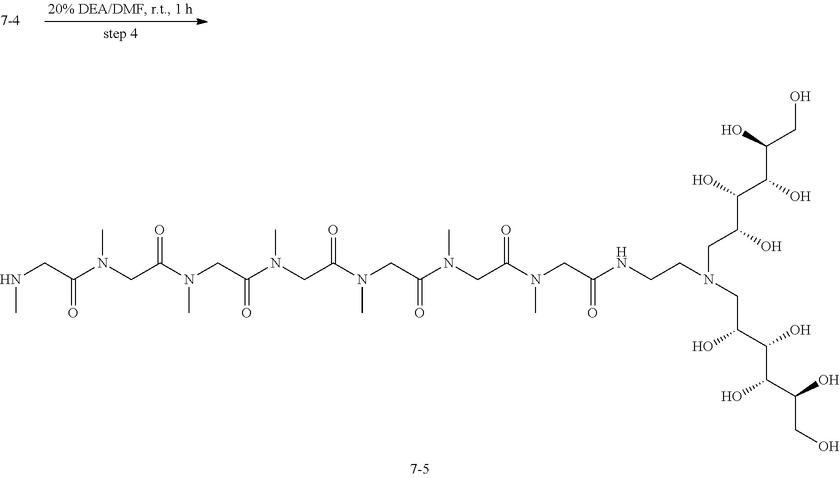 [SMILES: CNCC(=O)N(C)CC(=O)N(C)CC(=O)N(C)CC(=O)N(C)CC(=O)N(C)CC(=O)N(C)CC(=O)NCCN(C[C@@H](O)[C@H](O)[C@@H](O)[C@@H](O)CO)C[C@@H](O)[C@H](O)[C@@H](O)[C@@H](O)CO]

A solution of 7-4 (147 mg, 0.133 mmol) and DEA (1 mL) in anhydrous DMF (4 mL) was stirred at room temperature for 1 hr until LCMS indicated all starting amine was disappeared and desired product was detected. Then the solution was concentrated to dryness to 7-5 (103 mg, 0.116 mmol, 87.41%) as colorless oil, used as such in the next step.

### Step 5

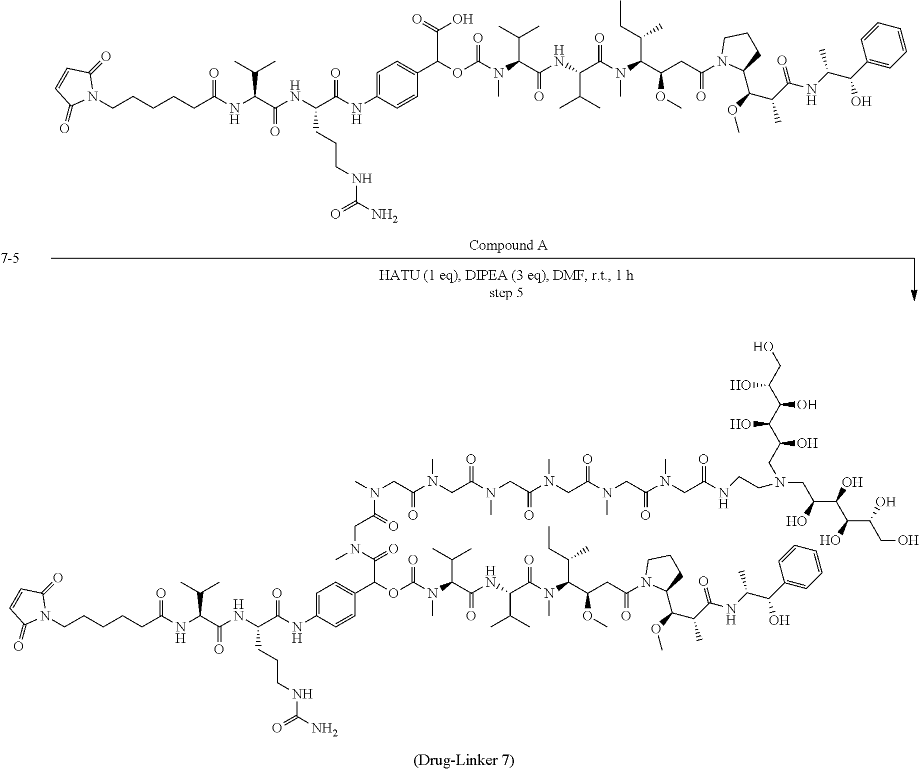

A solution of 7-5 (14 mg, 0.015 mmol), Compound A (20 mg, 0.015 mmol) and HATU (6 mg, 0.015 mmol) in anhydrous DMF (4 mL) was stirred at room temperature for 5 min, then DIPEA (6 mg, 0.045 mmol) was added. The resulting solution was stirred for another 1 hr at r.t. until LCMS indicated complete reaction. Then the reaction solution was purified by prep. HPLC to give Drug-Linker 7 (15 mg, 0.007 mmol, 44.87%) as a white solid. LCMS, m/z=1115.24 (M/2+H)⁺.

### Example 8: Preparation of Drug-Linker 8

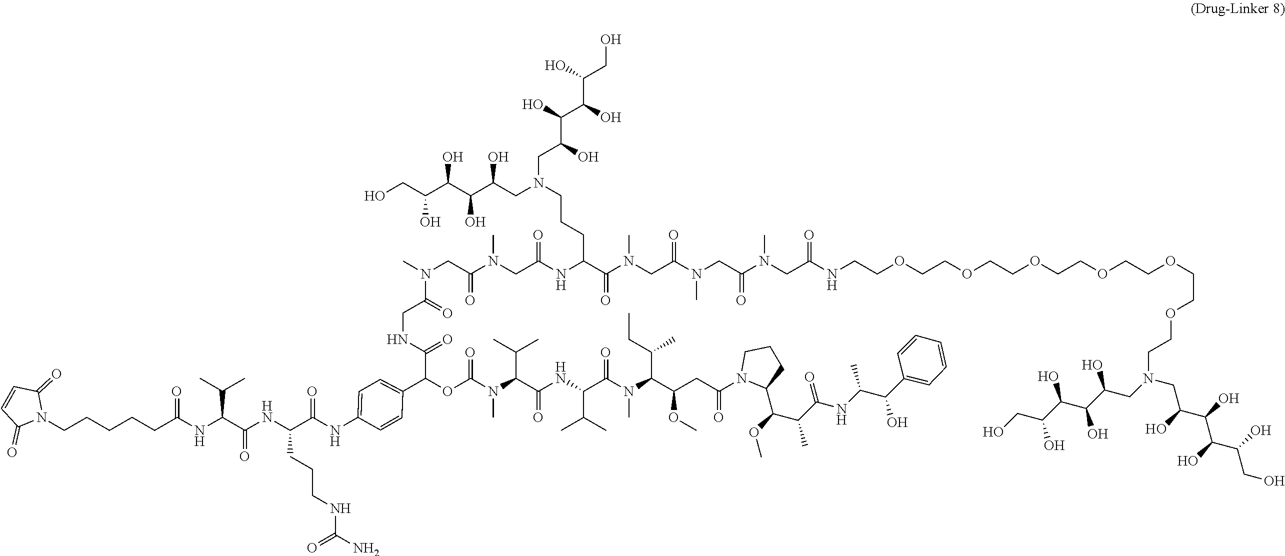 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCC(=O)N(C)CC(=O)N(C)CC(=O)NC(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

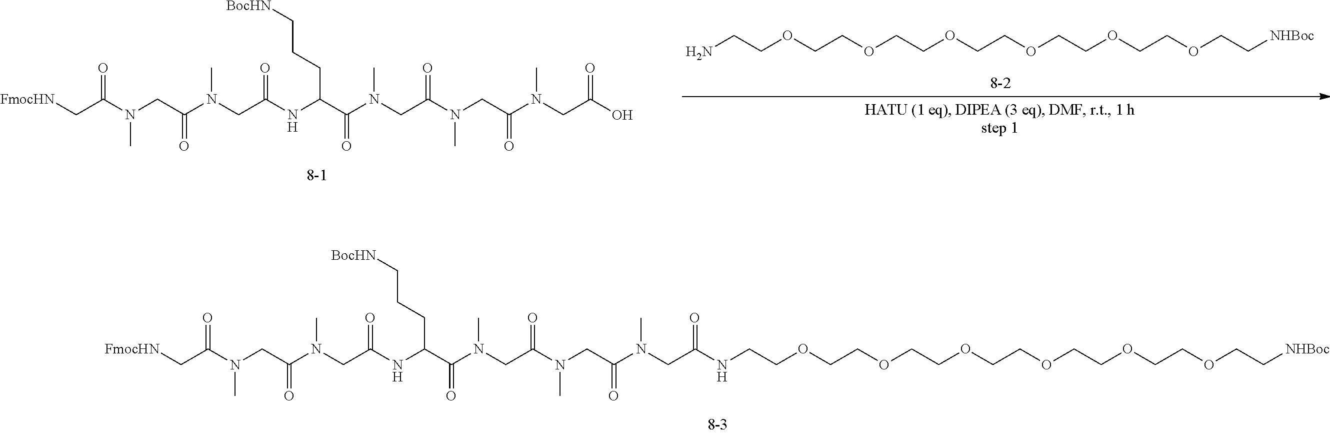

A solution of 8-1 (300 mg, 0.346 mmol), 8-2 (147 mg, 0.346 mmol) and HATU (132 mg, 0.346 mmol) in anhydrous DMF (5 mL) was stirred at room temperature for 5 min, then DIPEA (134 mg, 1.037 mmol) was added. The resulting solution was stirred for another 1 hr at r.t. until LCMS indicated complete reaction. The reaction solution was purified directly by reverse phase liquid chromatography to give 8-3 (310 mg, 0.243 mmol, 70.23%) as a white solid. Purity, 95%.

### Step 2

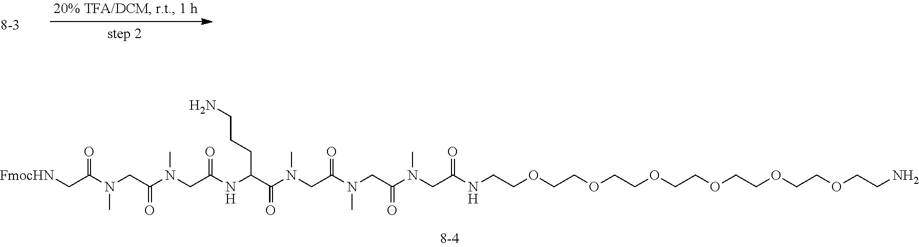

A solution of 8-3 (310 mg, 0.243 mmol) and TFA (1 mL) in anhydrous DCM (4 mL) was stirred at room temperature for 1 hr until LCMS indicated all starting amine was disappeared and desired product was detected. Then the solution was concentrated to dryness to 8-4 (255 mg, 0.238 mmol, 97.78%) as yellow oil, used as such in the next step. Purity, 95%.

### Step 3

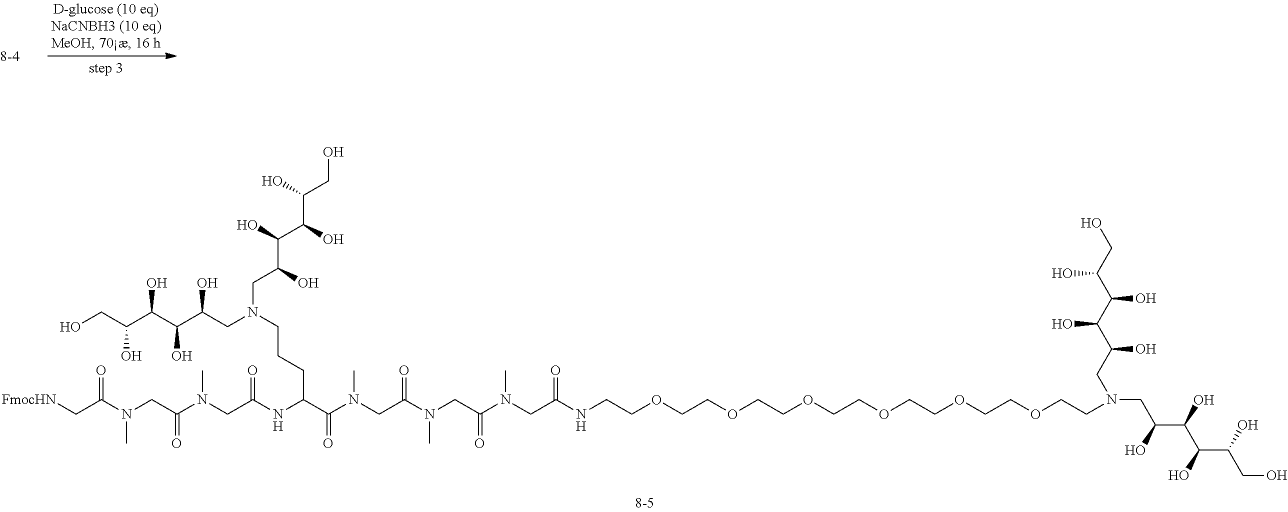

A solution of 8-4 (255 mg, 0.238 mmol) and D-Glucose (428 mg, 2.376 mmol) in anhydrous Methanol (50 mL) was heated at 50° C. for 30 min, then NaCNBH₃ (150 mg, 2.387 mmol) was added. The resulting solution was stirred for another 16 hr at 70° C. until indicated all starting amine was disappeared and the mass of desired product was detected. Then the reaction solution was concentrated and purified by reverse phase liquid chromatography to give 8-5 (185 mg, 0.107 mmol, 44.96%) as a white solid. Purity, 95%.

### Step 4

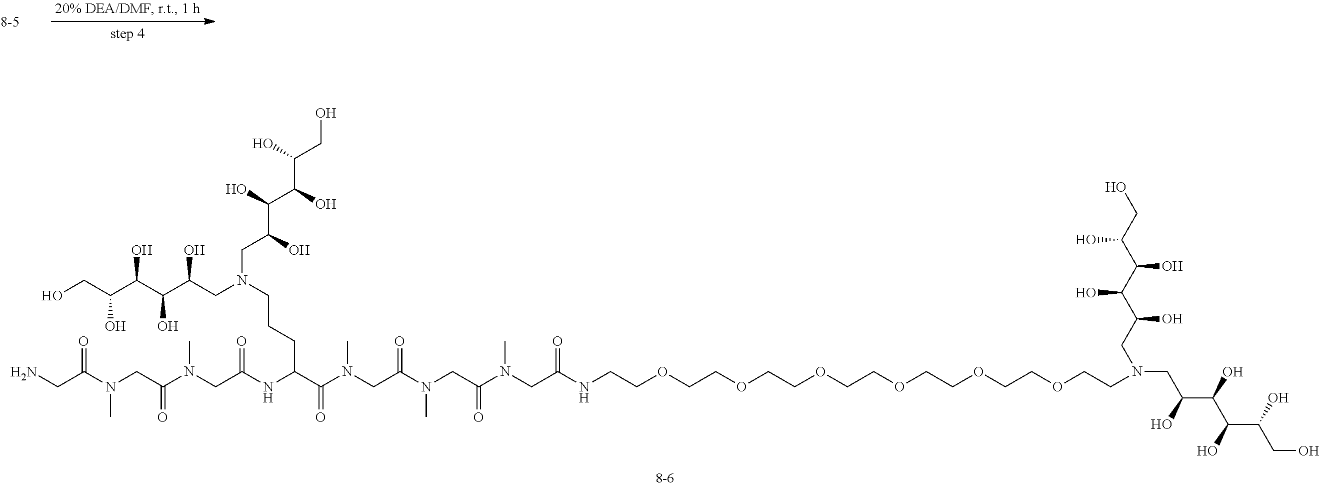 [SMILES: CN(CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)CN(C)C(=O)CN(C)C(=O)C(CCCN(C[C@H](O)[C@@H](O)[C@H](O)[C@H](O)CO)C[C@H](O)[C@@H](O)[C@H](O)[C@H](O)CO)NC(=O)CN(C)C(=O)CN(C)C(=O)CN]

A solution of 8-5 (185 mg, 0.107 mmol) and DEA (1 mL) in anhydrous DMF (4 mL) was stirred at room temperature for 1 hr until LCMS indicated all starting amine was disappeared and desired product was detected. Then the solution was concentrated to dryness to 8-6 (155 mg, 0.103 mmol, 96.08%) as colorless oil, used as such in the next step. Purity, 95%.

### Step 5

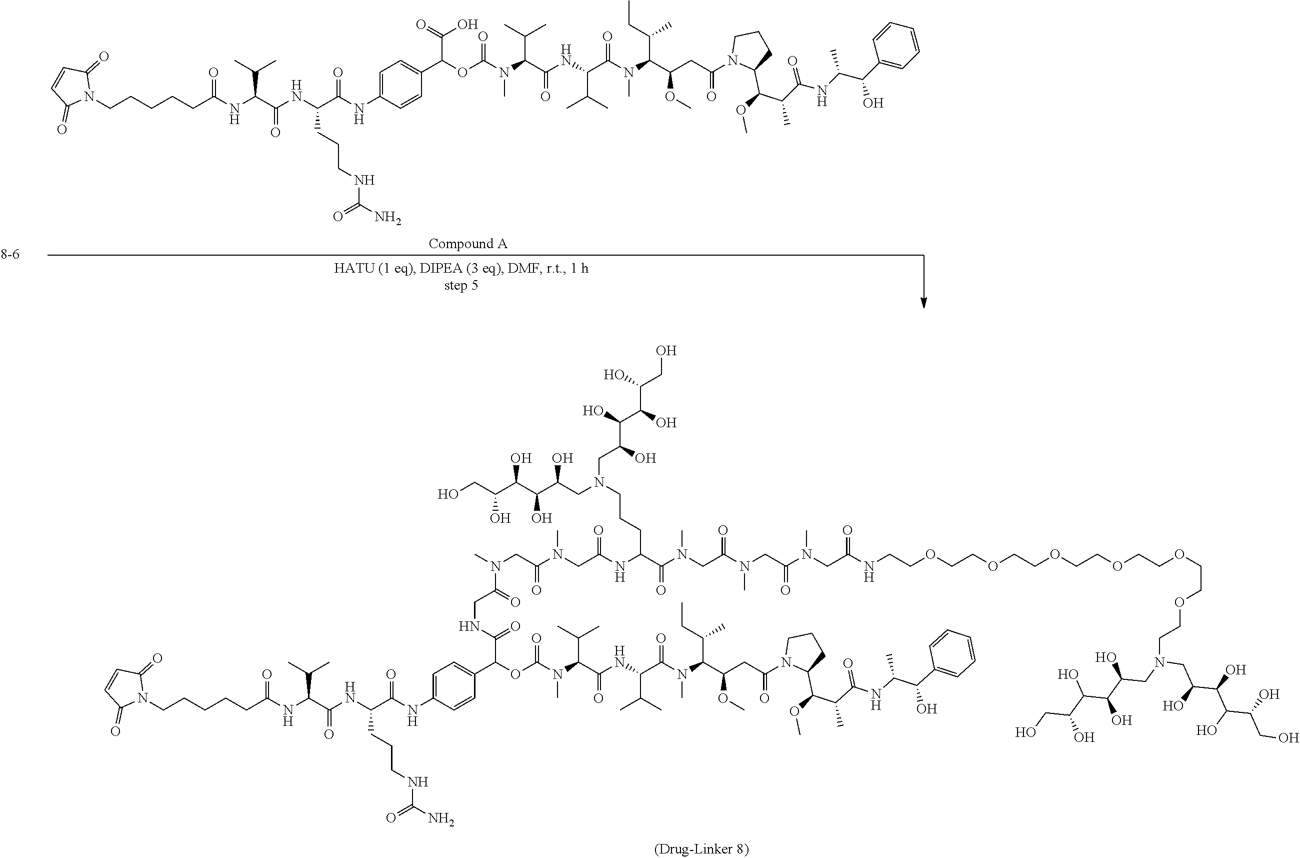

A solution of 8-6 (100 mg, 0.066 mmol), Compound A (90 mg, 0.066 mmol) and HATU (25 mg, 0.066 mmol) in anhydrous DMF (4 mL) was stirred at room temperature for 5 min, then DIPEA (26 mg, 0.201 mmol) was added. The resulting solution was stirred for another 1 hr at r.t. until LCMS indicated complete reaction. Then the reaction solution was purified by prep. HPLC to give Drug-Linker 8 (10 mg, 0.004 mmol, 5.32%) as a white solid. LCMS, m/z=1425.93 (M/2+H)⁺.

### Example 9: Preparation of Drug-Linker 9

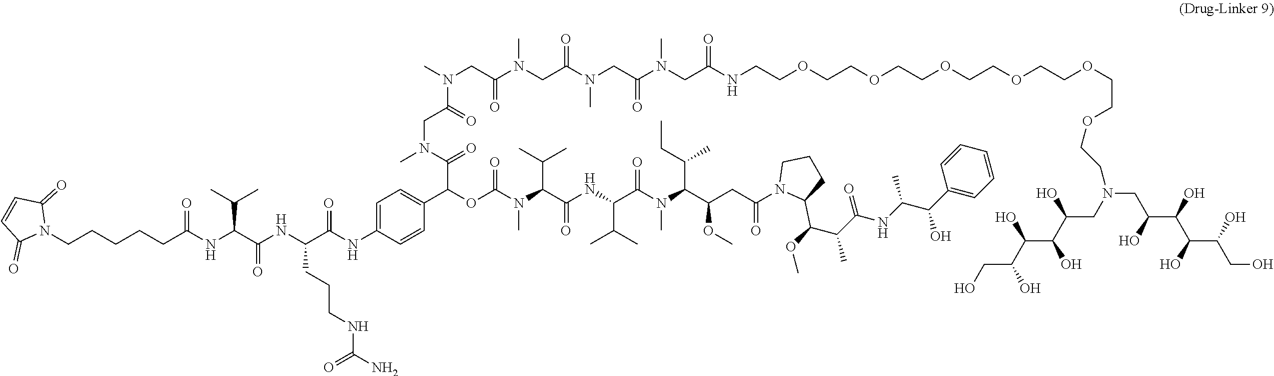 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)CC(=O)N(C)CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

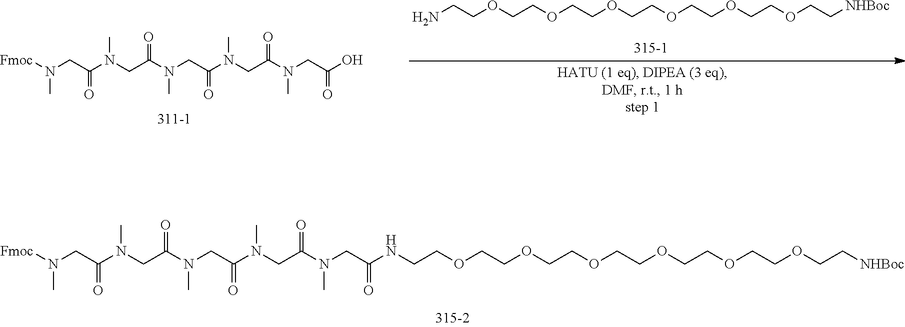

A solution of 311-1 (300 mg, 0.504 mmol), 315-1 (213 mg, 0.504 mmol) and HATU (192 mg, 0.407 mmol) in anhydrous DMF (10 mL) was stirred at room temperature for 5 men, then DIPEA (196 mg, 1.517 mmol) was added. The resulting solution was stirred for another 1 hr at r.t. until LCMS indicated complete reaction. The reaction solution was purified directly by reverse phase liquid chromatography to give 315-2 (315 mg, 0.314 mmol, 62.30%) as a white solid.

### Step 2

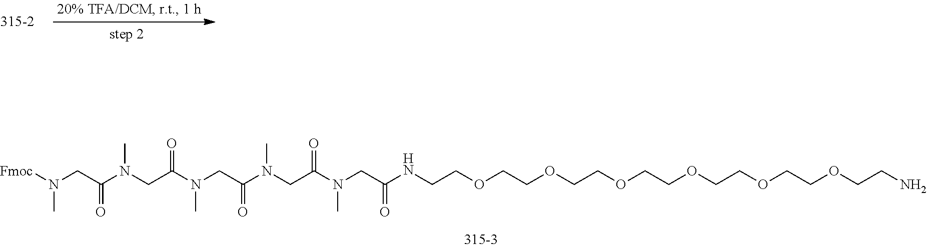

A solution of 315-2 (315 mg, 0.314 mmol) and TFA (2 mL) in anhydrous DCM (8 mL) was stirred at room temperature for 1 hr until LCMS indicated all starting amine was disappeared and desired product was detected. Then the solution was concentrated to dryness to 315-3 (264 mg, 0.293 mmol, 93.31%) as yellow oil, used as such in the next step.

### Step 3

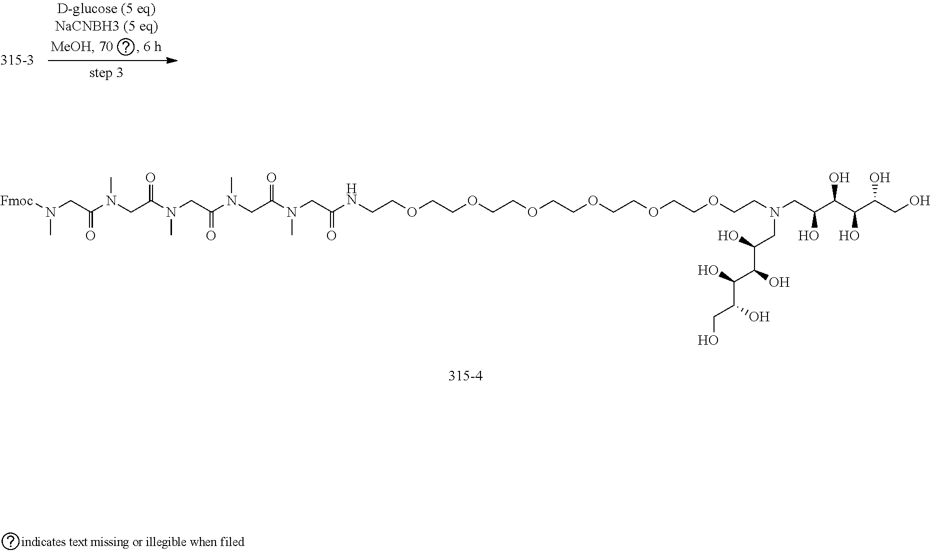

A solution of 315-3 (264 mg, 0.293 mmol) and D-Glucose (263 mg, 1.461 mmol) in anhydrous Methanol (50 mL) was heated at 50° C. for 30 min, then NaCNBH₃ (92 mg, 1.464 mmol) was added. The resulting solution was stirred for another 6 hr at 70° C. until indicated all starting amine was disappeared and the mass of desired product was detected. Then the reaction solution was concentrated and purified by reverse phase liquid chromatography to give 315-4 (233 mg, 0.189 mmol, 64.51%) as a white solid.

### Step 4

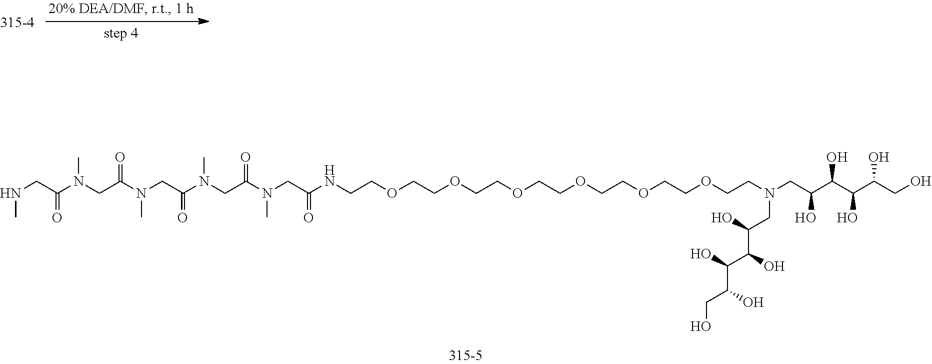 [SMILES: CNCC(=O)N(C)CC(=O)N(C)CC(=O)N(C)CC(=O)N(C)CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO]

A solution of 315-4 (233 mg, 0.189 mmol) and DEA (1 mL) in anhydrous DMF (4 mL) was stirred at room temperature for 1 hr until LCMS indicated all starting amine was disappeared and desired product was detected. Then the solution was concentrated to dryness to 315-5 (177 mg, 0.176 mmol, 93.12%) as colorless oil, used as such in the next step.

### Step 5

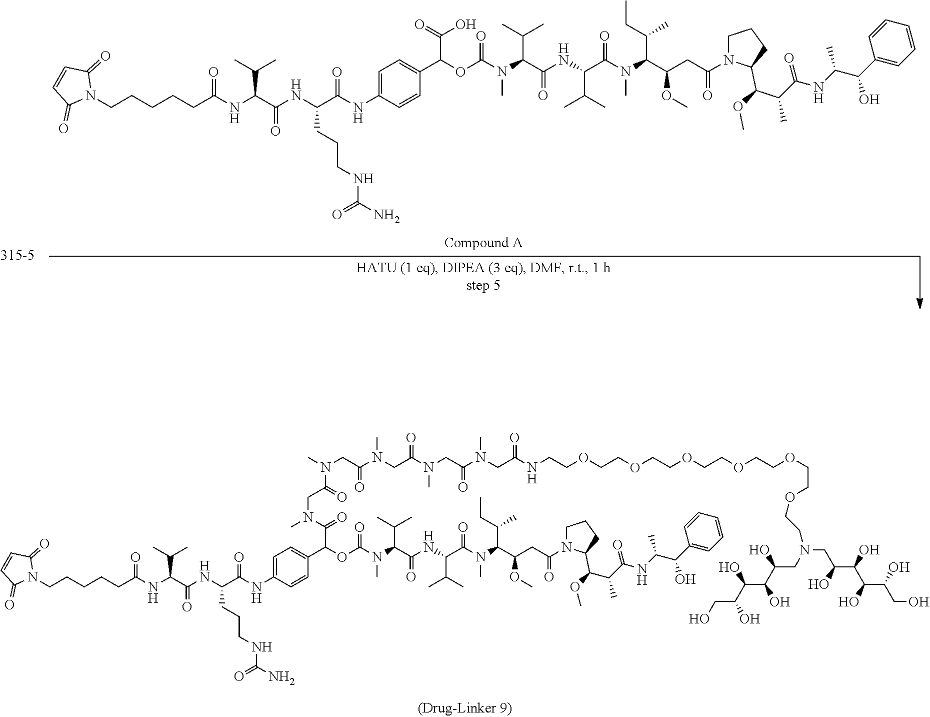

A solution of 315-5 (30 mg, 0.030 mmol), Compound A (40 mg, 0.030 mmol) and HATU (11 mg, 0.030 mmol) in anhydrous DMF (4 mL) was stirred at room temperature for 5 min, then DIPEA (11 mg, 0.085 mmol) was added. The resulting solution was stirred for another 1 hr at r.t. until LCMS indicated complete reaction. Then the reaction solution was purified by prep. HPLC to give Drug-Linker 8 (18 mg, 0.007 mmol, 25.53%) as a white solid. LCMS, m/z=1176.02 (M/2+H)⁺.

### Example 10: Preparation of Drug-Linker 10

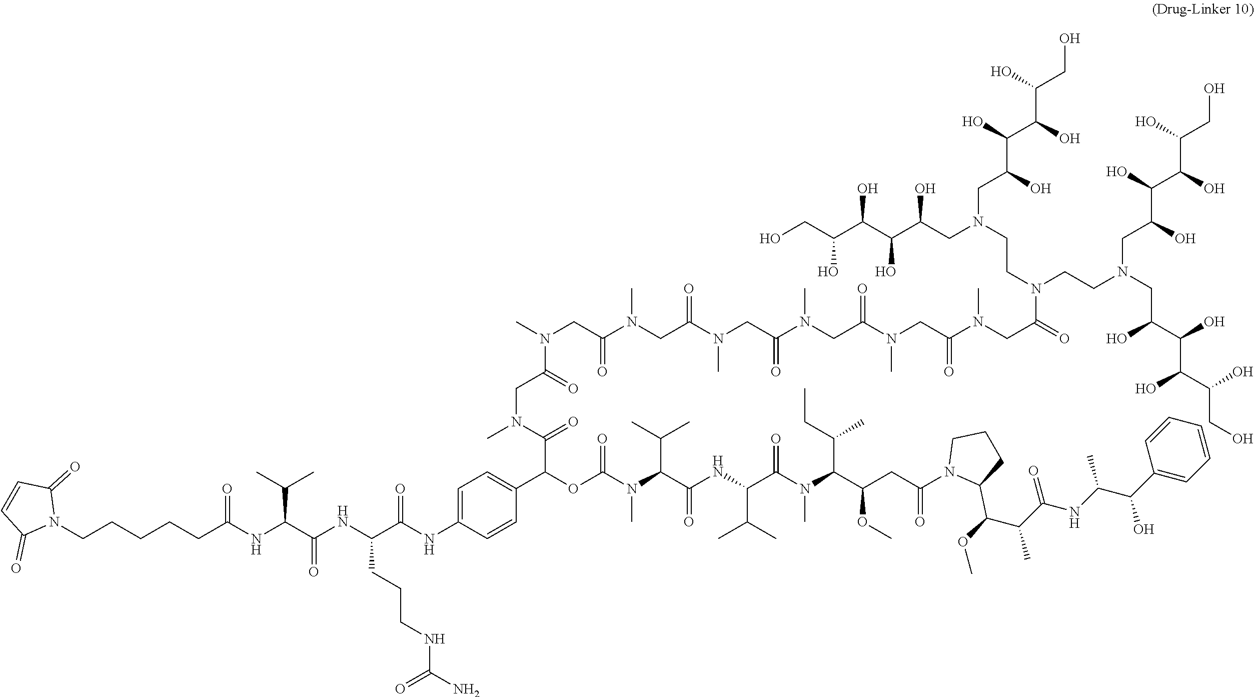 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)CC(=O)N(C)CC(=O)N(C)CC(=O)N(C)CC(=O)N(CCN(C[C@H](O)[C@@H](O)[C@H](O)[C@H](O)CO)C[C@H](O)[C@@H](O)[C@H](O)[C@H](O)CO)CCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

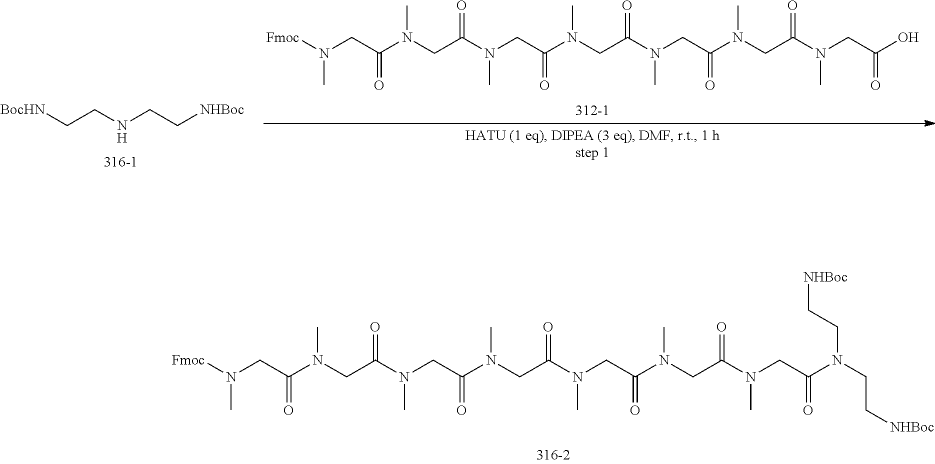

A solution of 316-1 (82 mg, 0.270 mmol), 312-1 (200 mg, 0.270 mmol) and HATU (103 mg, 0.270 mmol) in anhydrous DMF (10 mL) was stirred at room temperature for 5 min, then DIPEA (105 mg, 0.812 mmol) was added. The resulting solution was stirred for another 1 hr at r.t. until LCMS indicated complete reaction. The reaction solution was purified directly by reverse phase liquid chromatography to give 316-2 (205 mg, 0.200 mmol, 74.07%) as a white solid.

### Step 2

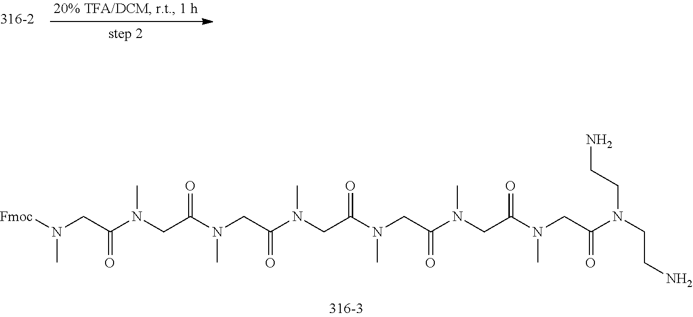

A solution of 316-2 (205 mg, 0.200 mmol) and TFA (1 mL) in anhydrous DCM (4 mL) was stirred at room temperature for 1 hr until LCMS indicated all starting amine was disappeared and desired product was detected. Then the solution was concentrated to dryness to 316-3 (157 mg, 0.191 mmol, 93.17%) as yellow oil, used as such in the next step.

### Step 3

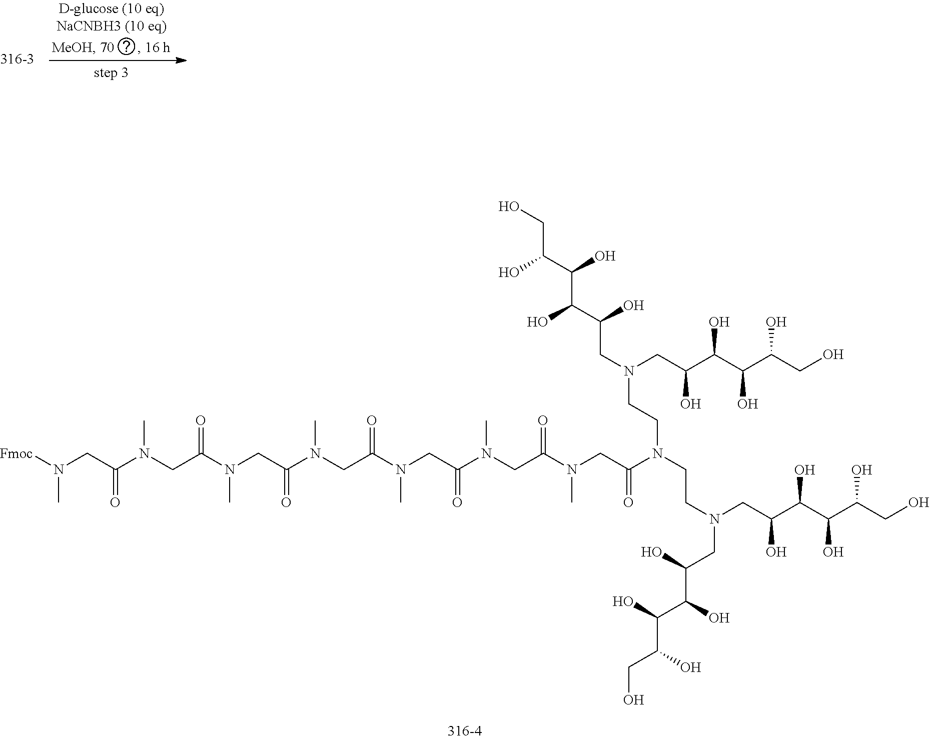

A solution of 316-3 (157 mg, 0.191 mmol) and D-Glucose (172 mg, 0.955 mmol) in anhydrous Methanol (50 mL) was heated at 50° C. for 30 min, then NaCNBH₃ (60 mg, 0.955 mmol) was added. The resulting solution was stirred for another 16 hr at 70° C. until indicated all starting amine was disappeared and the mass of desired product was detected. Then the reaction solution was concentrated and purified by reverse phase liquid chromatography to give 316-4 (135 mg, 0.091 mmol, 47.77%) as a white solid.

### Step 4

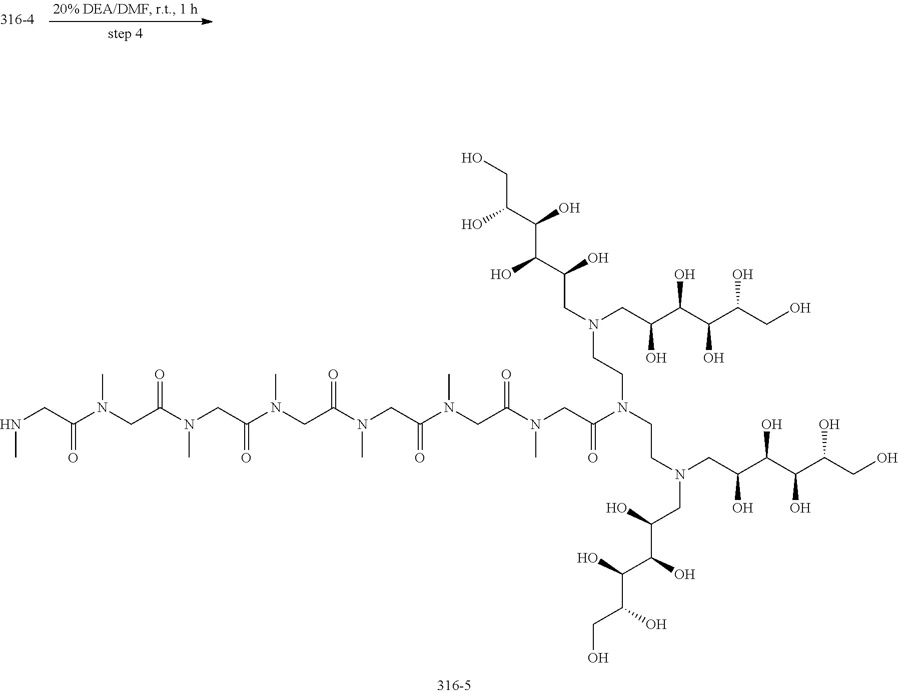 [SMILES: CNCC(=O)N(C)CC(=O)N(C)CC(=O)N(C)CC(=O)N(C)CC(=O)N(C)CC(=O)N(C)CC(=O)N(CCN(C[C@H](O)[C@@H](O)[C@H](O)[C@H](O)CO)C[C@H](O)[C@@H](O)[C@H](O)[C@H](O)CO)CCN(C[C@H](O)[C@@H](O)[C@H](O)[C@H](O)CO)C[C@H](O)[C@@H](O)[C@H](O)[C@H](O)CO]

A solution of 316-4 (135 mg, 0.091 mmol) and DEA (1 mL) in anhydrous DMF (4 mL) was stirred at room temperature for 1 hr until LCMS indicated all starting amine was disappeared and desired product was detected. Then the solution was concentrated to dryness to 316-5 (114 mg, 0.090 mmol, 98.90%) as colorless oil, used as such in the next step.

### Step 5

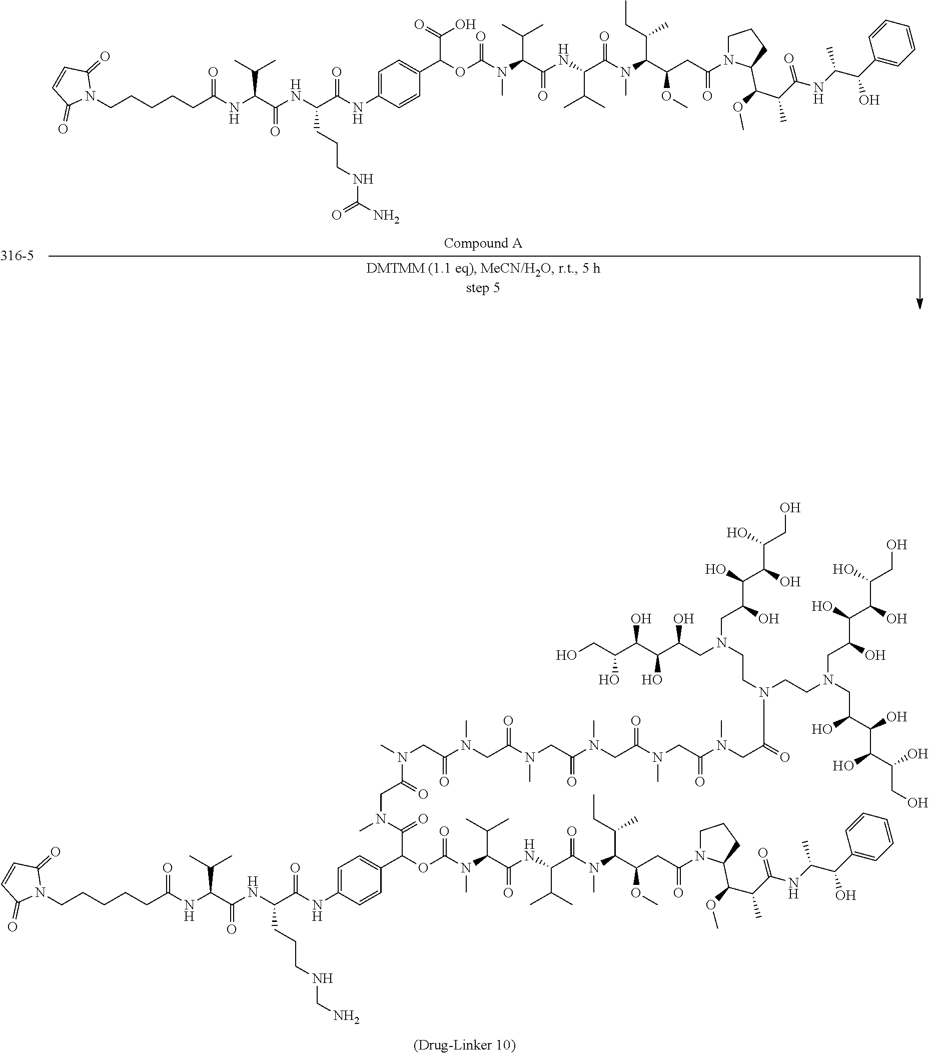

A solution of 316-5 (100 mg, 0.080 mmol), Compound A (108 mg, 0.080 mmol) and DMTMM (24 mg, 0.088 mmol) in H₂O (4 mL) and MeCN (4 mL) was stirred at room temperature for 5 hr. Then the reaction solution was purified by prep. HPLC to give Drug-Linker 10 (7 mg, 0.003 mmol, 3.37%) as a white solid. LCMS, m/z=1300.87 (M/2+H)⁺.

### Example 11: Preparation of Drug-Linker 11

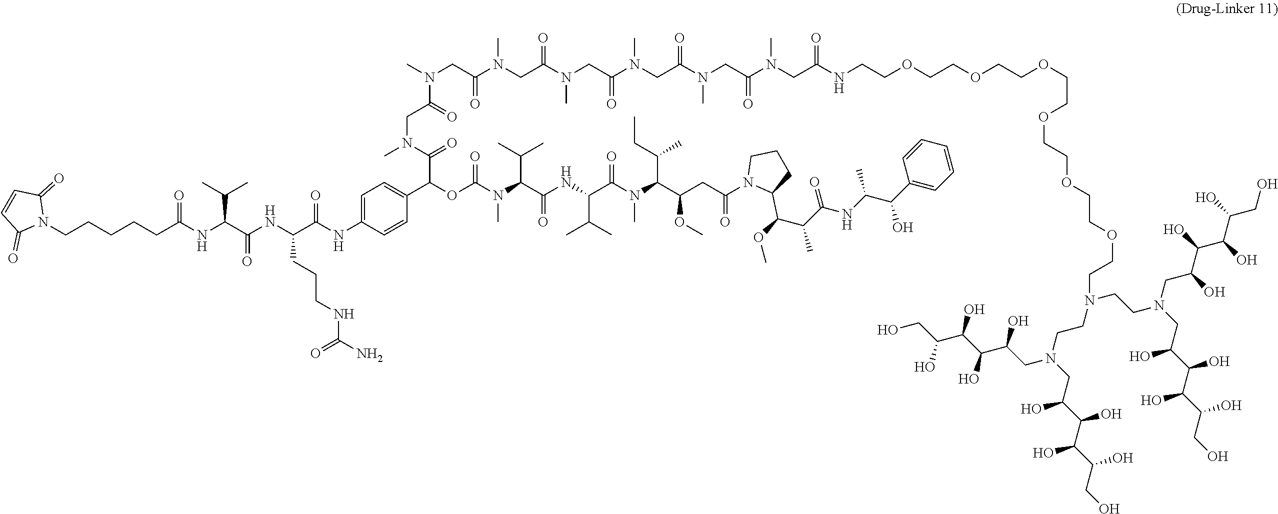 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)CC(=O)N(C)CC(=O)N(C)CC(=O)N(C)CC(=O)NCCOCCOCCOCCOCCOCCOCCN(CCN(C[C@H](O)[C@@H](O)[C@H](O)[C@H](O)CO)C[C@H](O)[C@@H](O)[C@H](O)[C@H](O)CO)CCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

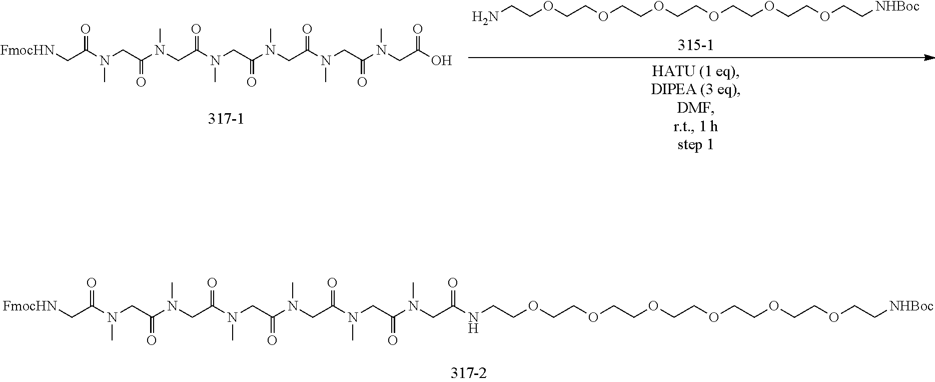

A solution of 317-1 (400 mg, 0.553 mmol), 315-1 (235 mg, 0.554 mmol) and HATU (210 mg, 0.553 mmol) in anhydrous DMF (6 mL) was stirred at room temperature for 5 min, then DIPEA (214 mg, 1.656 mmol) was added. The resulting solution was stirred for another 1 hr at room temperature until LCMS indicated complete reaction. The reaction solution was purified directly by reverse phase liquid chromatography to give 317-2 (410 mg, 0.363 mmol, 65.59%) as a white solid.

### Step 2

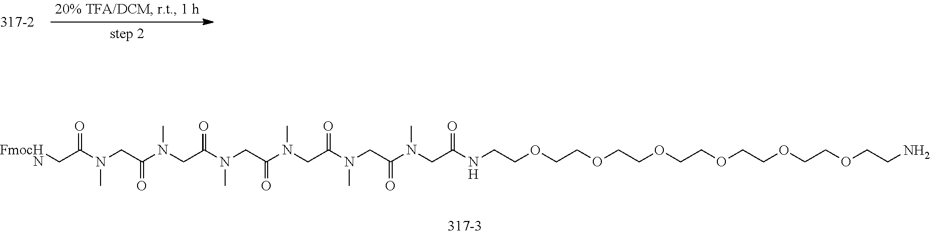

A solution of 317-2 (410 mg, 0.363 mmol) and TFA (2 mL) in anhydrous DCM (8 mL) was stirred at room temperature for 1 hr until LCMS indicated all starting amine was disappeared and desired product was detected. Then the solution was concentrated to dryness to 317-3 (365 mg, 0.354 mmol, 97.60%) as yellow oil, used as such in the next step.

### Step 3

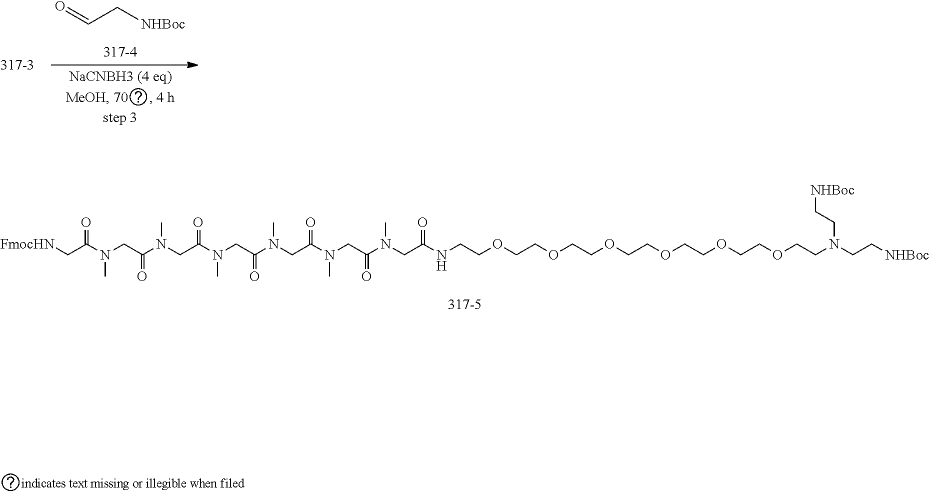

A solution of 317-3 (365 mg, 0.354 mmol) and 317-4 (338 mg, 2.123 mmol) in anhydrous Methanol (50 mL) was heated at 50° C. for 30 min, then NaCNBH₃ (134 mg, 2.132 mmol) was added. The resulting solution was stirred for another 4 hr at 70° C. until indicated all starting amine was disappeared and the mass of desired product was detected. Then the reaction solution was concentrated and purified by reverse phase liquid chromatography to give 317-5 (310 mg, 0.235 mmol, 66.51%) as a white solid.

### Step 4

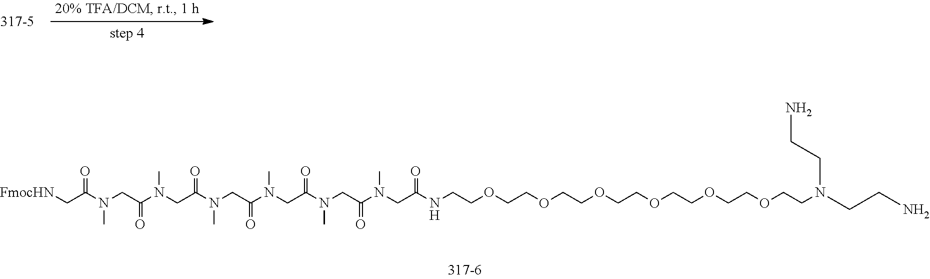

A solution of 317-5 (310 mg, 0.235 mmol) and TFA (2 mL) in anhydrous DCM (8 mL) was stirred at room temperature for 1 hr until LCMS indicated all starting amine was disappeared and desired product was detected. Then the solution was concentrated to dryness to 317-6 (250 mg, 0.224 mmol, 95.30%) as yellow oil, used as such in the next step.

### Step 5

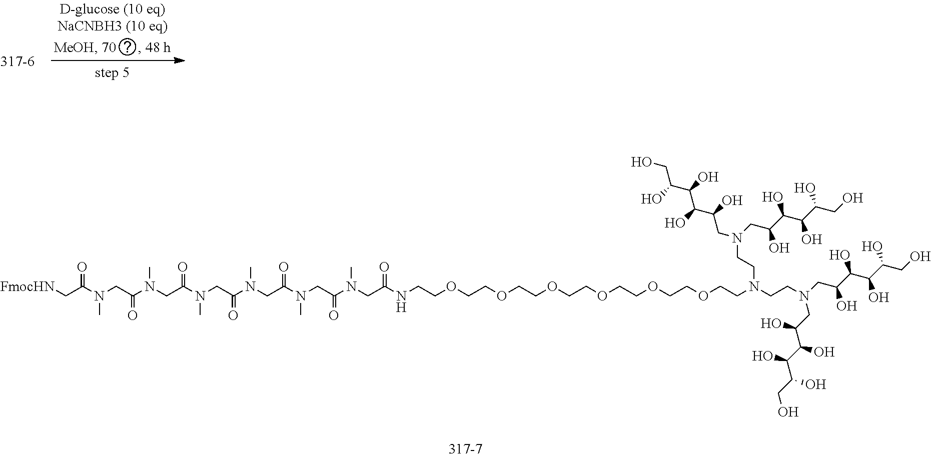

A solution of 317-6 (250 mg, 0.224 mmol) and D-Glucose (484 mg, 2.687 mmol) in anhydrous Methanol (50 mL) was heated at 50° C. for 30 min, then NaCNBH₃ (169 mg, 2.689 mmol) was added. The resulting solution was stirred for another 48 hr at 70° C. until indicated all starting amine was disappeared and the mass of desired product was detected. Then the reaction solution was concentrated and purified by reverse phase liquid chromatography to give 317-7 (174 mg, 0.098 mmol, 43.81%) as a white solid.

### Step 6

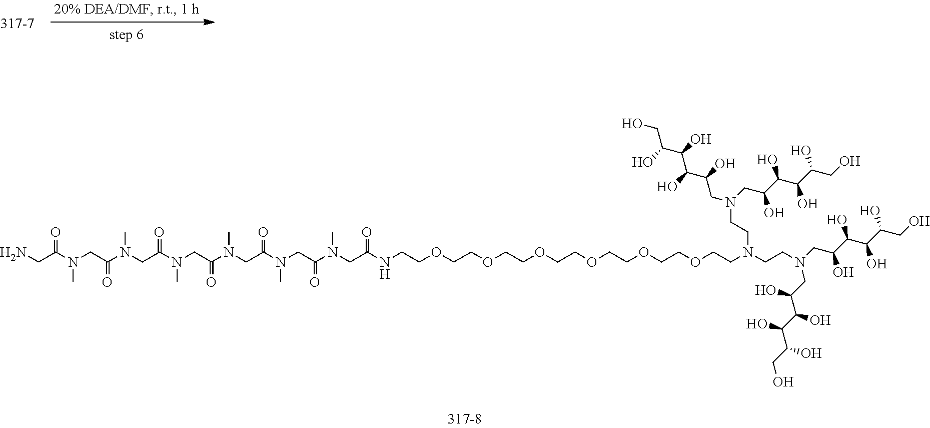 [SMILES: CN(CC(=O)NCCOCCOCCOCCOCCOCCOCCN(CCN(C[C@H](O)[C@@H](O)[C@H](O)[C@H](O)CO)C[C@H](O)[C@@H](O)[C@H](O)[C@H](O)CO)CCN(C[C@H](O)[C@@H](O)[C@H](O)[C@H](O)CO)C[C@H](O)[C@@H](O)[C@H](O)[C@H](O)CO)C(=O)CN(C)C(=O)CN(C)C(=O)CN(C)C(=O)CN(C)C(=O)CN(C)C(=O)CN]

A solution of 317-7 (174 mg, 0.098 mmol) and DEA (1 mL) in anhydrous DMF (4 mL) was stirred at room temperature for 1 hr until LCMS indicated all starting amine was disappeared and desired product was detected. Then the solution was concentrated to dryness to 317-8 (145 mg, 0.094 mmol, 95.41%) as colorless oil, used as such in the next step.

### Step 7

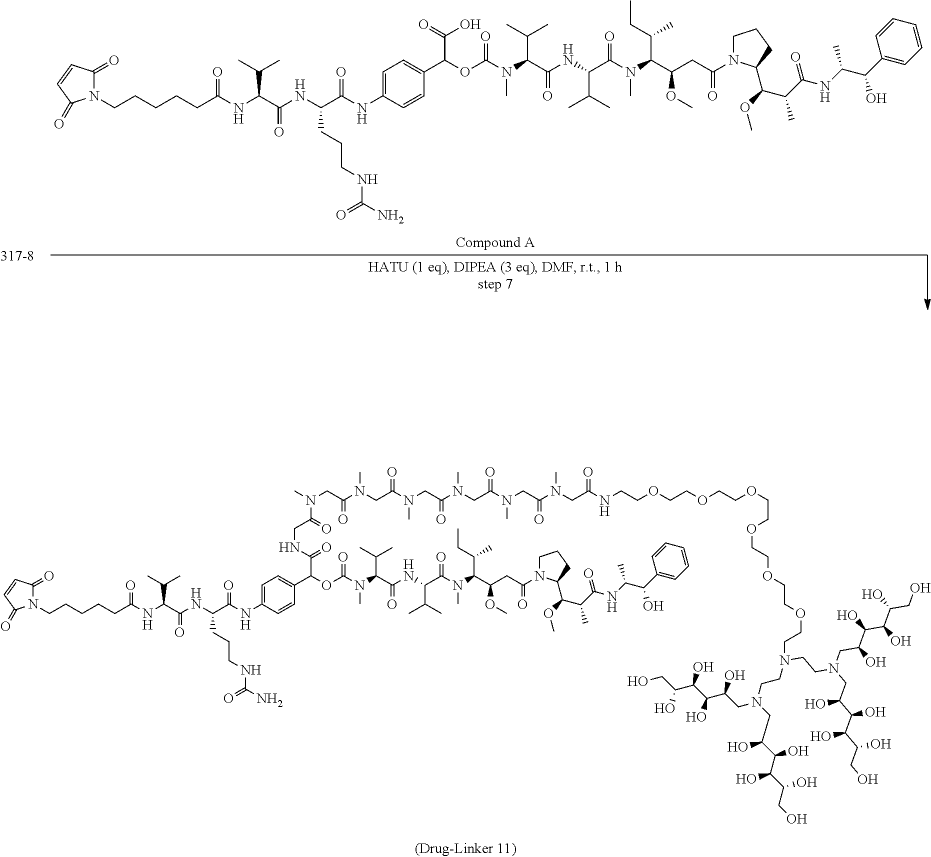

A solution of 317-8 (80 mg, 0.052 mmol), Compound A (70 mg, 0.052 mmol) and HATU (20 mg, 0.052 mmol) in anhydrous DMF (4 mL) was stirred at room temperature for 5 min, the DIPEA (20 mg, 0.155 mmol) was added. The resulting solution was stirred for another 1 hr at r.t. until LCMS indicated complete reaction. Then the reaction solution was purified by prep. HPLC to give Drug-Linker 11 (40 mg, 0.014 mmol, 26.59%) as a white solid. LCMS, m/z=1447.20 (M/2+H⁺).

### Example 12: Preparation of Drug-Linker 12

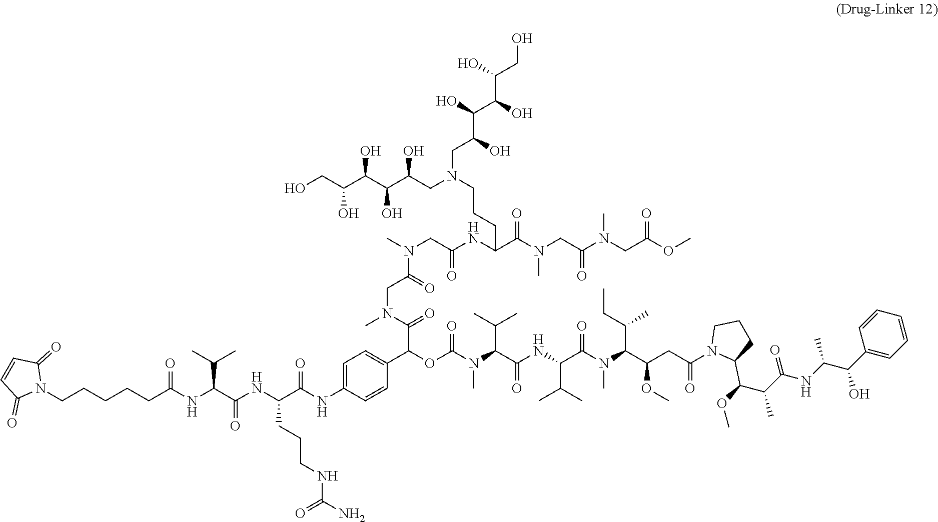 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)NC(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)OC)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

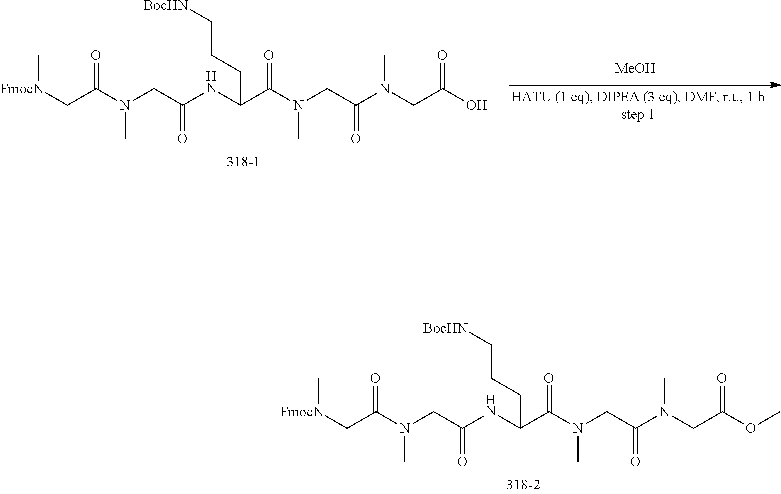

A solution of 318-1 (200 mg, 0.271 mmol), MeOH (43 mg, 1.342 mmol) and HATU (103 mg, 0.271 mmol) in anhydrous DMF (10 mL) was stirred at room temperature for 5 min, then DIPEA (105 mg, 0.812 mmol) was added. The resulting solution was stirred for another 1 hr at r.t. until LCMS indicated complete reaction. The reaction solution was purified directly by reverse phase liquid chromatography to give 318-2 (167 mg, 0.222 mmol, 81.92%) as a white solid.

### Step 2

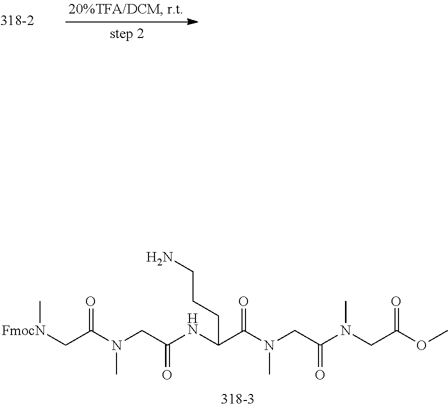

A solution of 318-2 (167 mg, 0.222 mmol) and TFA (1 mL) in anhydrous DCM (4 mL) was stirred at room temperature for 1 hr until LCMS indicated all starting amine was disappeared and desired product was detected. Then the solution was concentrated to dryness to 318-3 (135 mg, 0.207 mmol, 93.24%) as yellow oil, used as such in the next step.

### Step 3

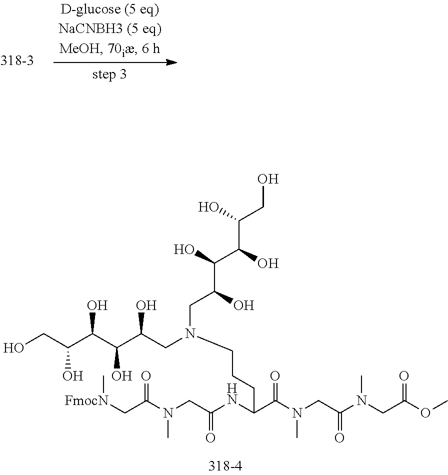

A solution of 318-3 (135 mg, 0.207 mmol) and D-Glucose (186 mg, 1.032 mmol) in anhydrous Methanol (50 mL) was heated at 50° C. for 30 min, then NaCNBH₃ (65 mg, 1.034 mmol) was added. The resulting solution was stirred for another 6 hr at 70° C. until indicated all starting amine was disappeared and the mass of desired product was detected. Then the reaction solution was concentrated and purified by reverse phase liquid chromatography to give 318-4 (121 mg, 0.123 mmol, 59.42%) as a white solid.

### Step 4

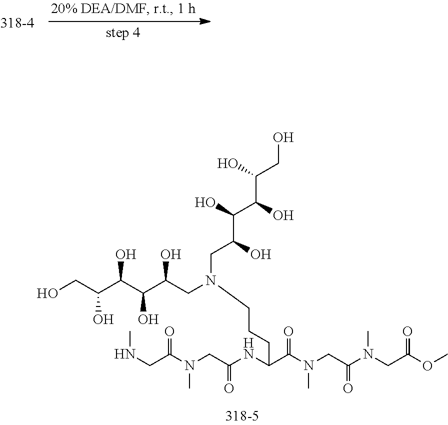 [SMILES: CNCC(=O)N(C)CC(=O)NC(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)OC]

A solution of 318-4 (121 mg, 0.123 mmol) and DEA (1 mL) in anhydrous DMF (4 mL) was stirred at room temperature for 1 hr until LCMS indicated all starting amine was disappeared and desired product was detected. Then the solution was concentrated to dryness to 318-5 (75 mg, 0.099 mmol, 80.49%) as colorless oil, used as such in the next step.

### Step 5

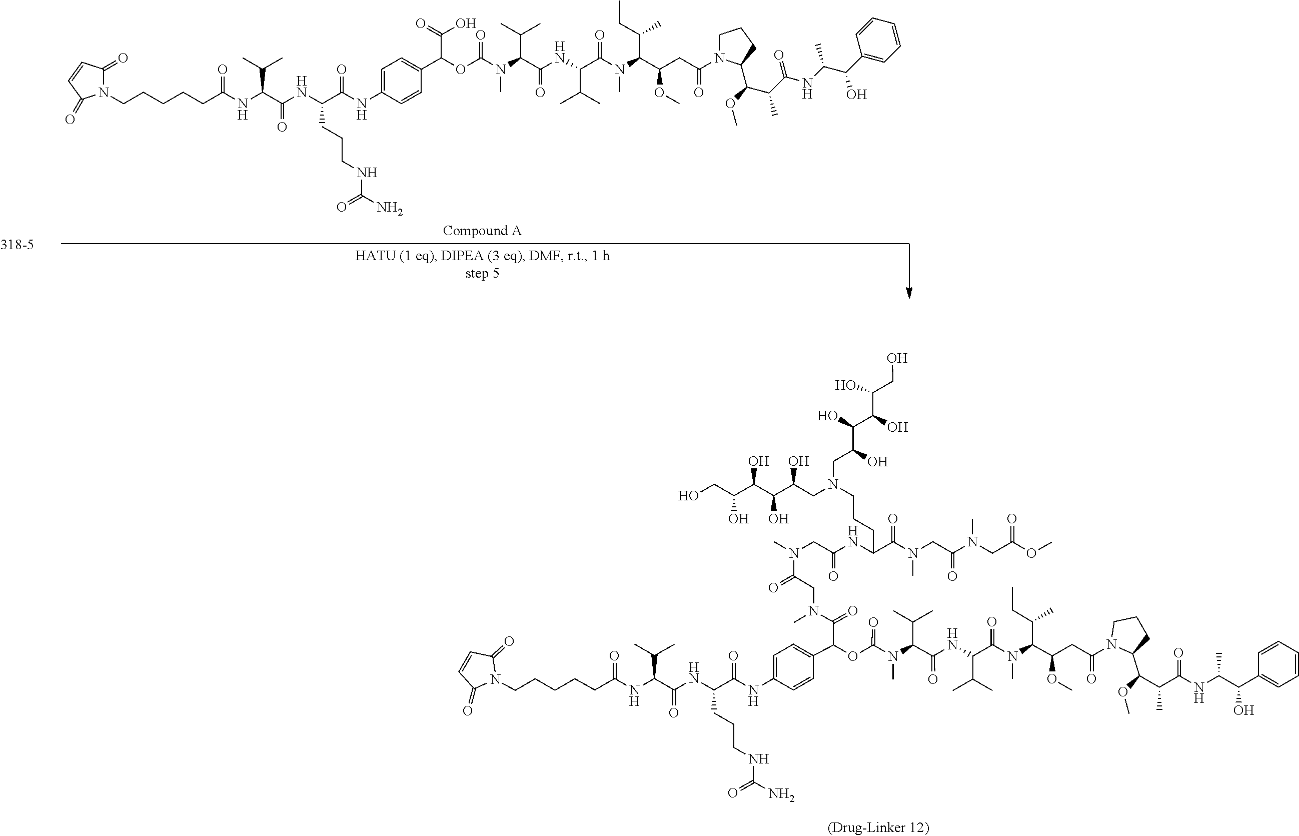

A solution of 318-5 (17 mg, 0.022 mmol), Compound A (30 mg, 0.022 mmol) and HATU (8 mg, 0.022 mmol) in anhydrous DMF (4 mL) was stirred at room temperature for 5 min, then DIPEA (8 mg, 0.062 mmol) was added. The resulting solution was stirred for another 1 hr at r.t. until LCMS indicated complete reaction. Then the reaction solution was purified by prep. HPLC to give Drug-Linker 12 (16 mg, 0.008 mmol, 36.36%) as a white solid. LCMS, m/z=1051.28 (M/2+H)⁺.

### Example 13: Preparation of Drug-Linker 13

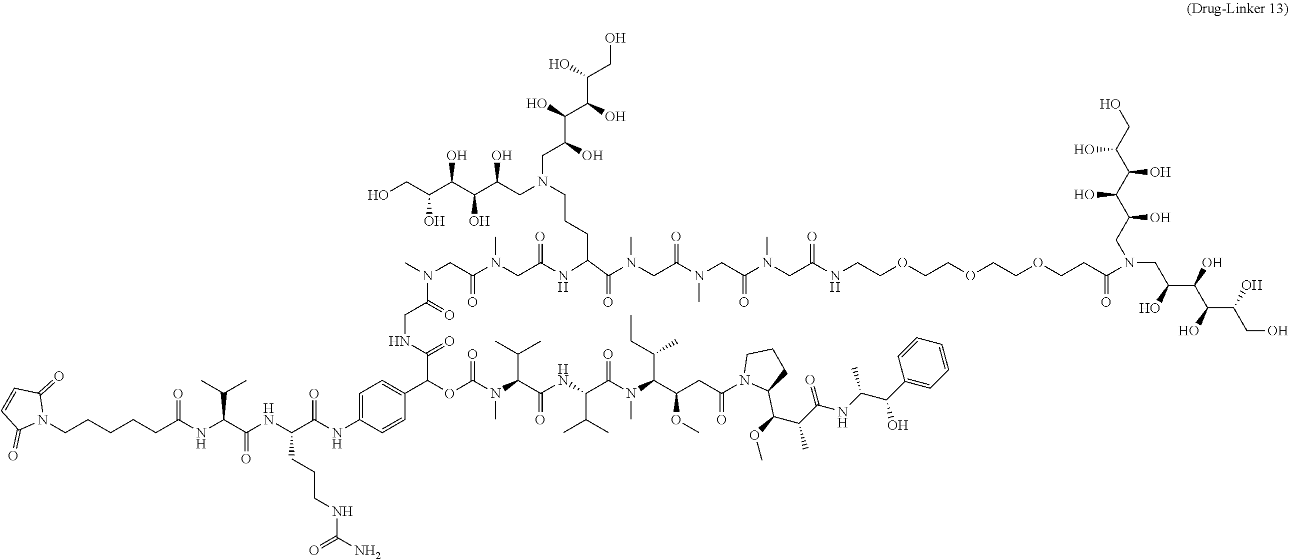 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCC(=O)N(C)CC(=O)N(C)CC(=O)NC(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)NCCOCCOCCOCCC(=O)N(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

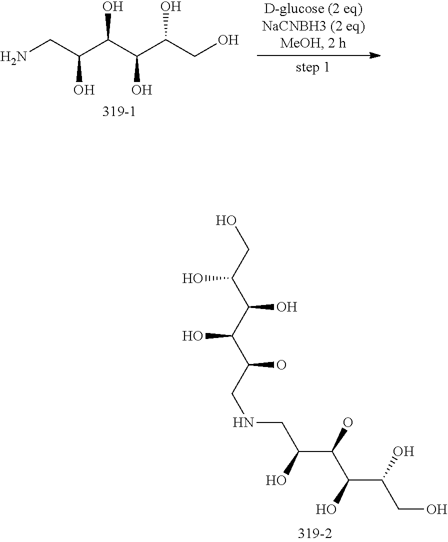

A solution of 319-1 (2.0 g, 0.011 mol) and D-Glucose (4.0 g, 0.022 mol) in anhydrous Methanol (50 mL) was heated at 50° C. for 30 mi, then NaCNBH₃ (1.4 g, 0.022 mol) was added. The resulting solution was stirred for another 2 hr at 70° C. until indicated all starting amine was disappeared and the mass of desired product was detected. Then the reaction solution was concentrated and purified by reverse phase liquid chromatography to give 319-2 (2.6 g, 7.529 mmol, 68.44%) as a white solid.

### Step 2

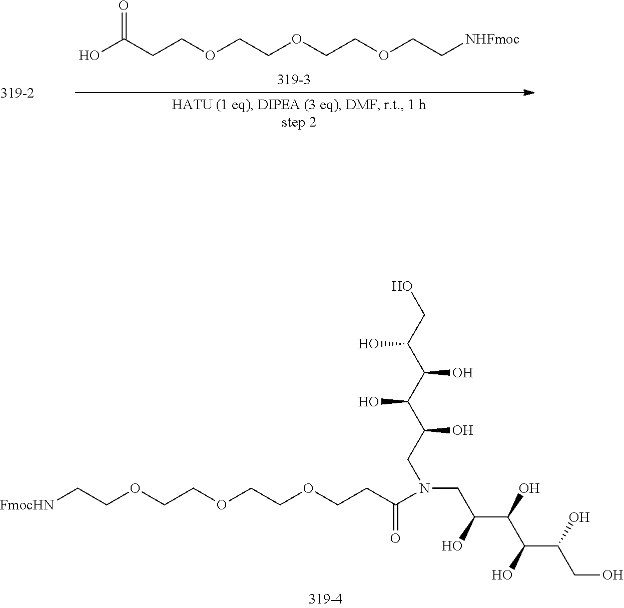

A solution of 319-2 (2.6 g, 7.529 mmol), 319-3 (3.3 g, 7.529 mmol) and HATU (2.9 g, 7.529 mmol) in anhydrous DMF (10 mL) was stirred at room temperature for 5 min, then DIPEA (2.9 g, 22.586 mmol) was added. The resulting solution was stirred for another 1 hr at r.t. until LCMS indicated complete reaction. The reaction solution was purified directly by reverse phase liquid chromatography to give 319-4 (1.5 g, 1.946 mmol, 25.85%) as a white solid.

### Step 3

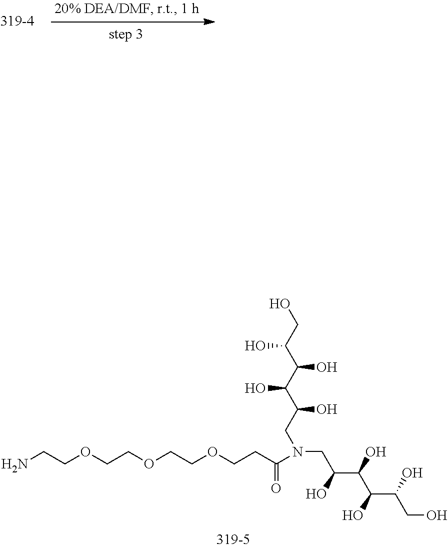 [SMILES: NCCOCCOCCOCCC(=O)N(C[C@H](O)[C@@H](O)[C@H](O)[C@H](O)CO)C[C@H](O)[C@@H](O)[C@H](O)[C@H](O)CO]

A solution of 319-4 (1.5 g, 1.946 mmol) and DEA (2 mL) in anhydrous DMF (8 mL) was stirred at room temperature for 1 hr until LCMS indicated all starting amine was disappeared and desired product was detected. Then the solution was concentrated to dryness to 319-5 (950 mg, 1.732 mmol, 89.00%) as colorless oil, used as such in the next step.

### Step 4

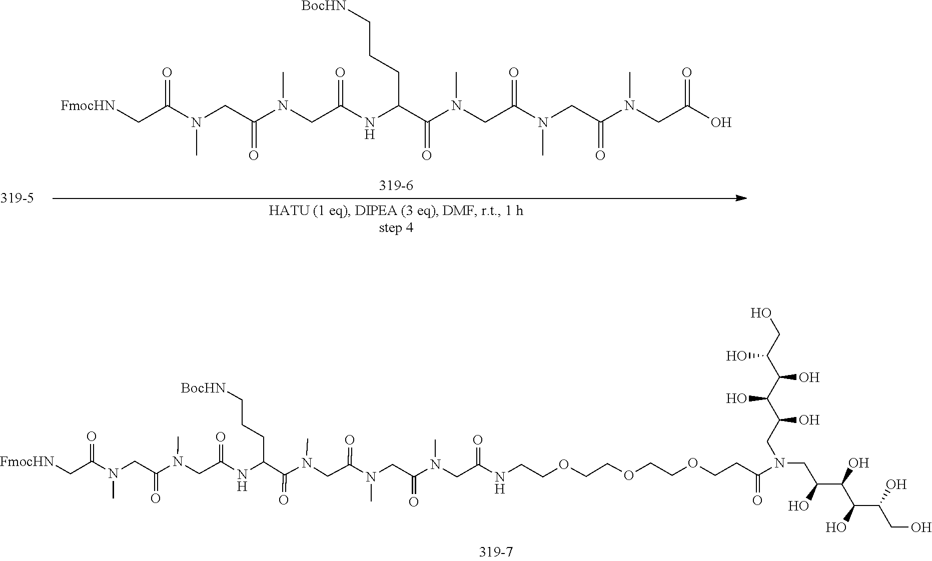

A solution of 319-5 (950 mg, 1.732 mmol), 319-6 (1501 mg, 1.732 mmol) and HATU (658 mg, 1.732 mmol) in anhydrous DMF (10 mL) was stirred at room temperature for 5 min, then DIPEA (670 mg, 5.184 mmol) was added. The resulting solution was stirred for another 1 hr at r.t. until LCMS indicated complete reaction. The reaction solution was purified directly by reverse phase liquid chromatography to give 319-7 (700 mg, 0.501 mmol, 28.93%) as a white solid.

### Step 5

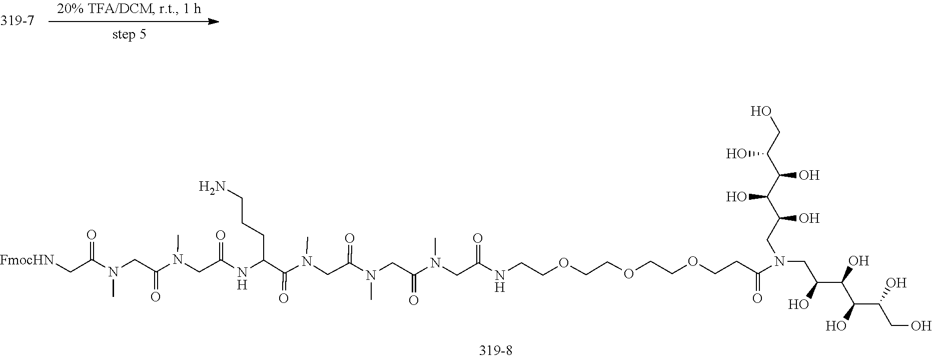

A solution of 319-7 (300 mg, 0.215 mmol) and TFA (1 mL) in anhydrous DCM (4 mL) was stirred at room temperature for 1 hr until LCMS indicated all starting amine was disappeared and desired product was detected. Then the solution was concentrated to dryness to 319-8 (245 mg, 0.189 mmol, 87.91%) as yellow oil, used as such in the next step.

### Step 6

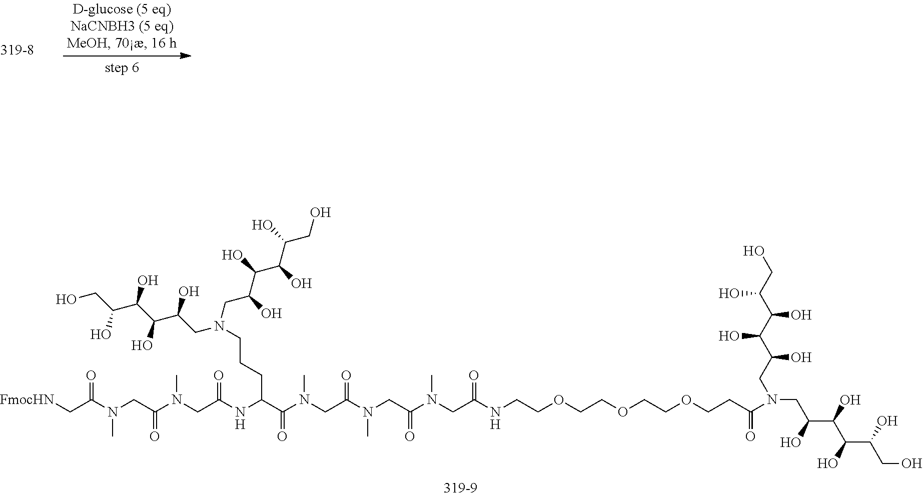

A solution of 319-8 (245 mg, 0.189 mmol) and D-Glucose (170 mg, 0.945 mmol) in anhydrous Methanol (50 mL) was heated at 50° C. for 30 min, then NaCNBH₃ (60 mg, 0.945 mmol) was added. The resulting solution was stirred for another 16 hr at 70° C. until indicated all starting amine was disappeared and the mass of desired product was detected. Then the reaction solution was concentrated and purified by reverse phase liquid chromatography to give 319-9 (140 mg, 0.086 mmol, 45.50%) as a white solid.

### Step 7

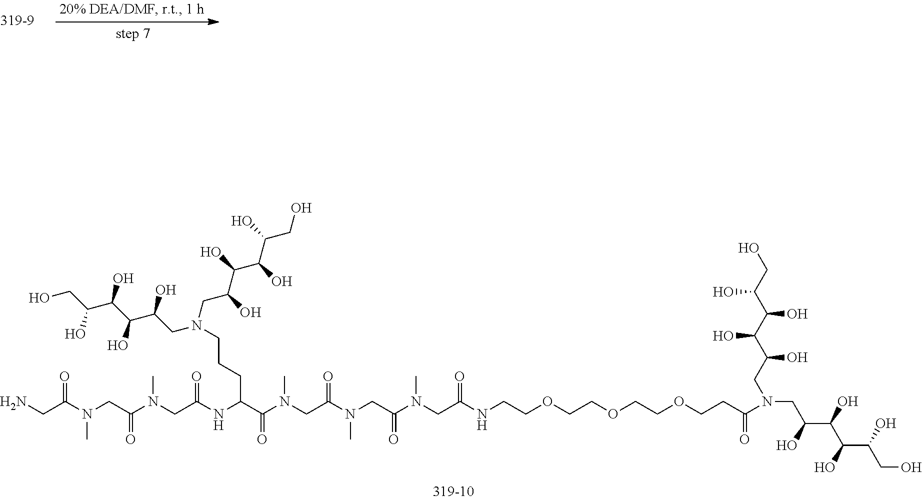 [SMILES: CN(CC(=O)NCCOCCOCCOCCC(=O)N(C[C@H](O)[C@@H](O)[C@H](O)[C@H](O)CO)C[C@H](O)[C@@H](O)[C@H](O)[C@H](O)CO)C(=O)CN(C)C(=O)CN(C)C(=O)C(CCCN(C[C@H](O)[C@@H](O)[C@H](O)[C@H](O)CO)C[C@H](O)[C@@H](O)[C@H](O)[C@H](O)CO)NC(=O)CN(C)C(=O)CN(C)C(=O)CN]

A solution of 319-9 (140 mg, 0.086 mmol) and DEA (1 mL) in anhydrous DMF (4 mL) was stirred at room temperature for 1 hr until LCMS indicated all starting amine was disappeared and desired product was detected. Then the solution was concentrated to dryness to 319-10 (112 mg, 0.080 mmol, 93.02%) as colorless oil, used as such in the next step.

### Step 8

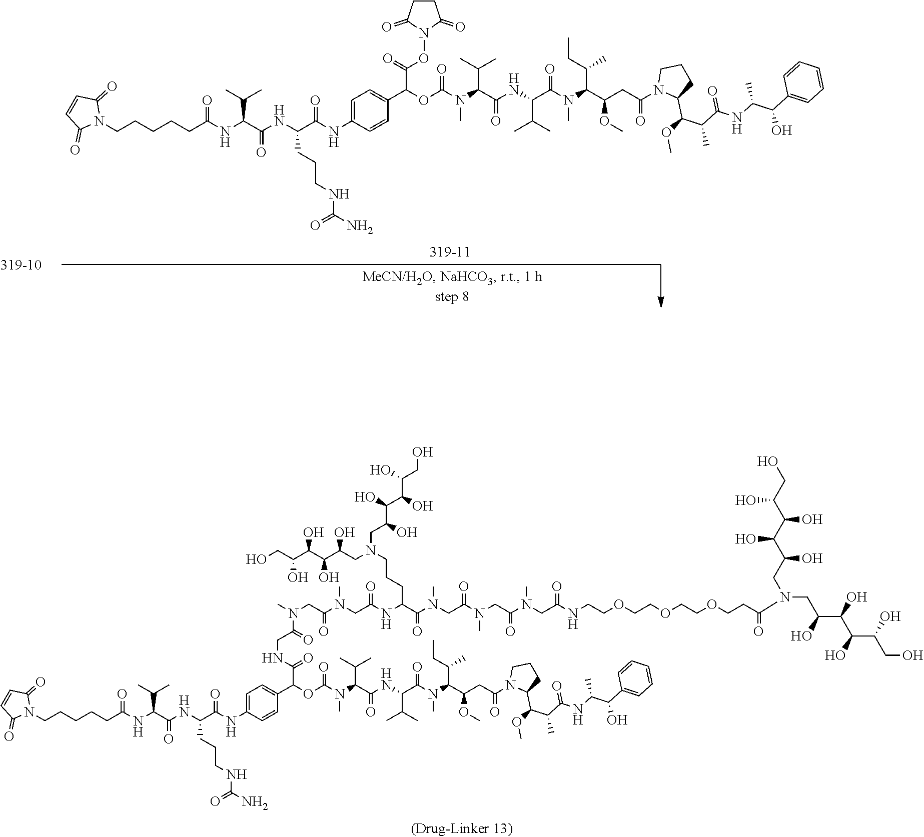

A solution of 319-10 (112 mg, 0.080 mmol) and 319-11 (116 mg, 0.080 mmol) in H₂O (3 mL) and MeCN (3 mL) was adjusted to pH=8 nu aqueous sodium bicarbonate. The resulting solution was stirred for another 1 hr at r.t. until LCMS indicated complete reaction. Then the reaction solution was purified by prep. HPLC to give Drug-Linker 13 (8 mg, 0.003 mmol, 3.75%) as a white solid. LCMS, m/z=916.23 (M/3+H)⁺.

### Example 14: Preparation of Drug-Linker 14

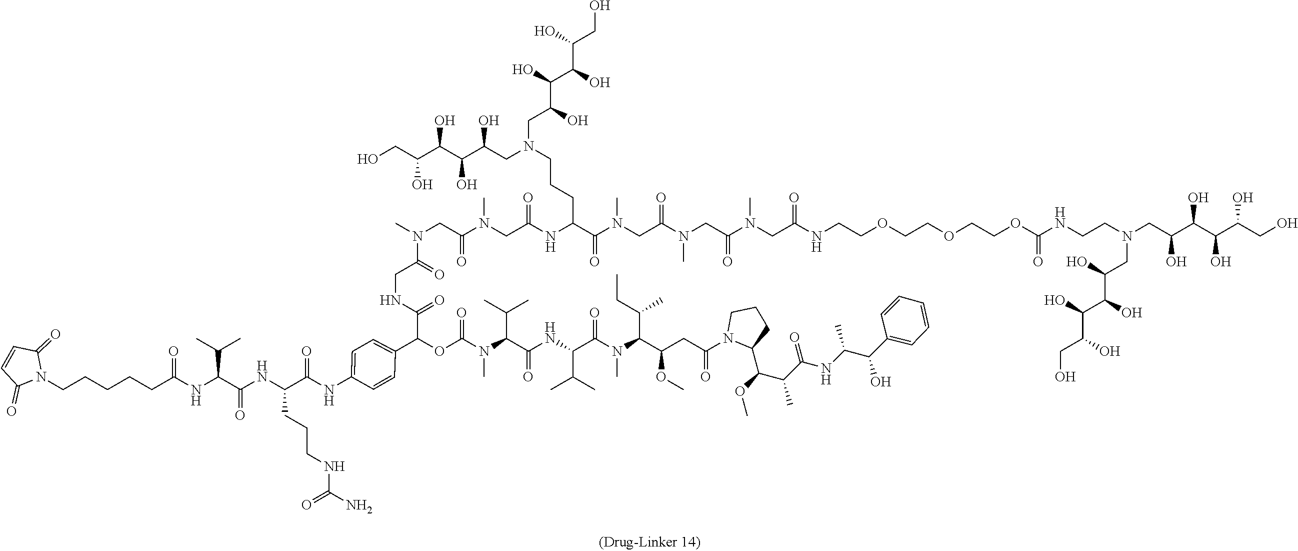 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCC(=O)N(C)CC(=O)N(C)CC(=O)NC(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)NCCOCCOCCOC(=O)NCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

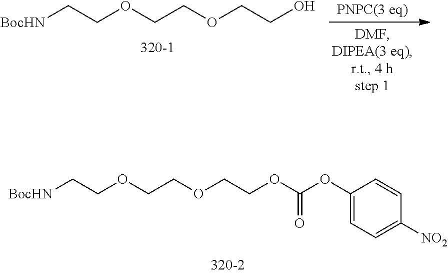 [SMILES: CC(C)(C)OC(=O)NCCOCCOCCO ; CC(C)(C)OC(=O)NCCOCCOCCOC(=O)Oc1ccc([N+](=O)[O-])cc1]

A solution of 320-1 (1.0 g, 4.011 mol) and DIPEA (1.6 g, 12.380 mol) in anhydrous DMF (15 mL) was stirred at room temperature for 5 min, then PNPC (3.7 g, 12.163 mmol) was added. The resulting solution was stirred for another 4 hr at r.t. until LCMS indicated all starting amine was disappeared and desired product was detected. Then the reaction solution was concentrated and purified by reverse phase liquid chromatography to give 320-2 (943 mg, 2.276 mmol, 56.73%) as a yellow solid.

### Step 2

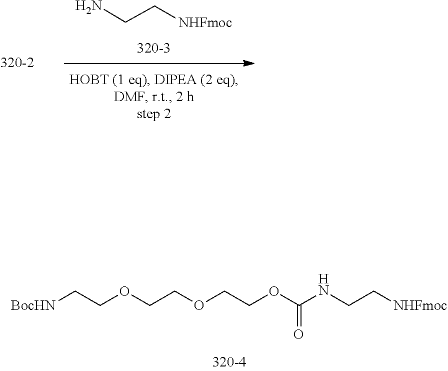

A solution of 320-2 (943 mg, 2.276 mmol), 320-3 (642 mg, 2.274 mmol), and HOBT (307 mg, 2.274 mmol) in anhydrous DMF (50 mL) was stirred at room temperature, then DIPEA (588 mg, 4.550 mmol) was added. The resulting solution was stirred for another 1 hr at r.t. until LCMS indicated all starting amine was disappeared and desired product was detected. The reaction solution was purified directly by reverse phase liquid chromatography to give 320-4 (850 mg, 1.524 mmol, 66.97%) as a white solid.

### Step 3

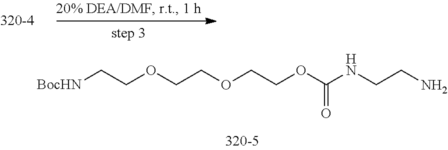 [SMILES: CC(C)(C)OC(=O)NCCOCCOCCOC(=O)NCCN]

A solution of 320-4 (850 mg, 1.524 mmol) and DEA (2 mL) in anhydrous DMF (8 mL) was stirred at room temperature for 1 hr until LCMS indicated all starting amine was disappeared and desired product was detected. Then the solution was concentrated to dryness to 320-5 (491 mg, 1.464 mmol, 96.06%) as colorless oil, used as such in the next step.

### Step 4

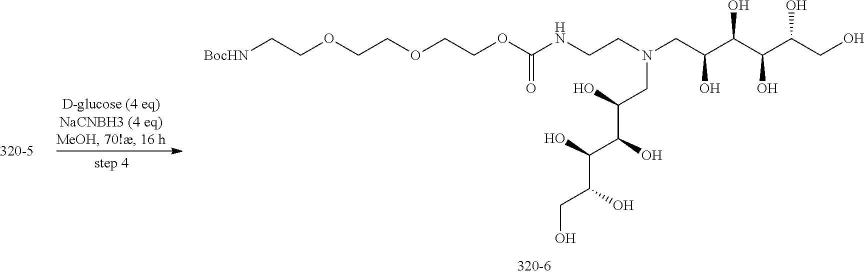 [SMILES: CC(C)(C)OC(=O)NCCOCCOCCOC(=O)NCCN(C[C@H](O)[C@@H](O)[C@H](O)[C@H](O)CO)C[C@H](O)[C@@H](O)[C@H](O)[C@H](O)CO]

A solution of 320-5 (491 mg, 1.464 mmol) and D-Glucose (1.05 g, 5.828 mmol) in anhydrous Methanol (50 mL) was heated at 50° C. for 30 min, then NaCNBH₃ (368 mg, 5.856 mmol) was added. The resulting solution was stirred for another 16 hr at 70′C until indicated all starting amine was disappeared and the mass of desired product was detected. Then the reaction solution was concentrated and purified by reverse phase liquid chromatography to give 320-6 (470 mg, 0.708 mmol, 48.37%) as a white solid.

### Step 5

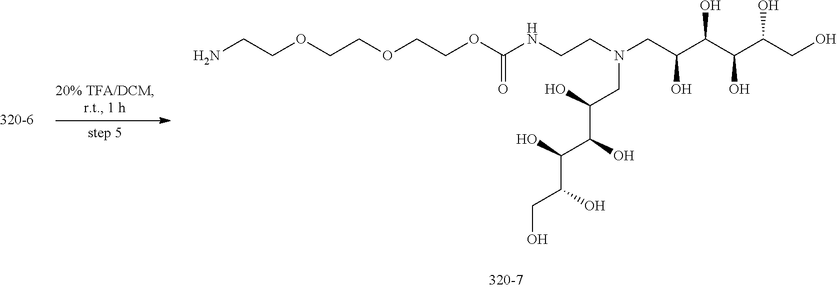 [SMILES: NCCOCCOCCOC(=O)NCCN(C[C@H](O)[C@@H](O)[C@H](O)[C@H](O)CO)C[C@H](O)[C@@H](O)[C@H](O)[C@H](O)CO]

A solution of 320-6 (470 mg, 0.708 mmol) and TFA (2 mL) in anhydrous DCM (8 mL) was stirred at room temperature for 1 hr until LCMS indicated all starting amine was disappeared and desired product was detected. Then the solution was concentrated to dryness to 320-7 (385 mg, 0.683 mmol, 96.48%) as yellow oil, used as such in the next step.

### Step 6

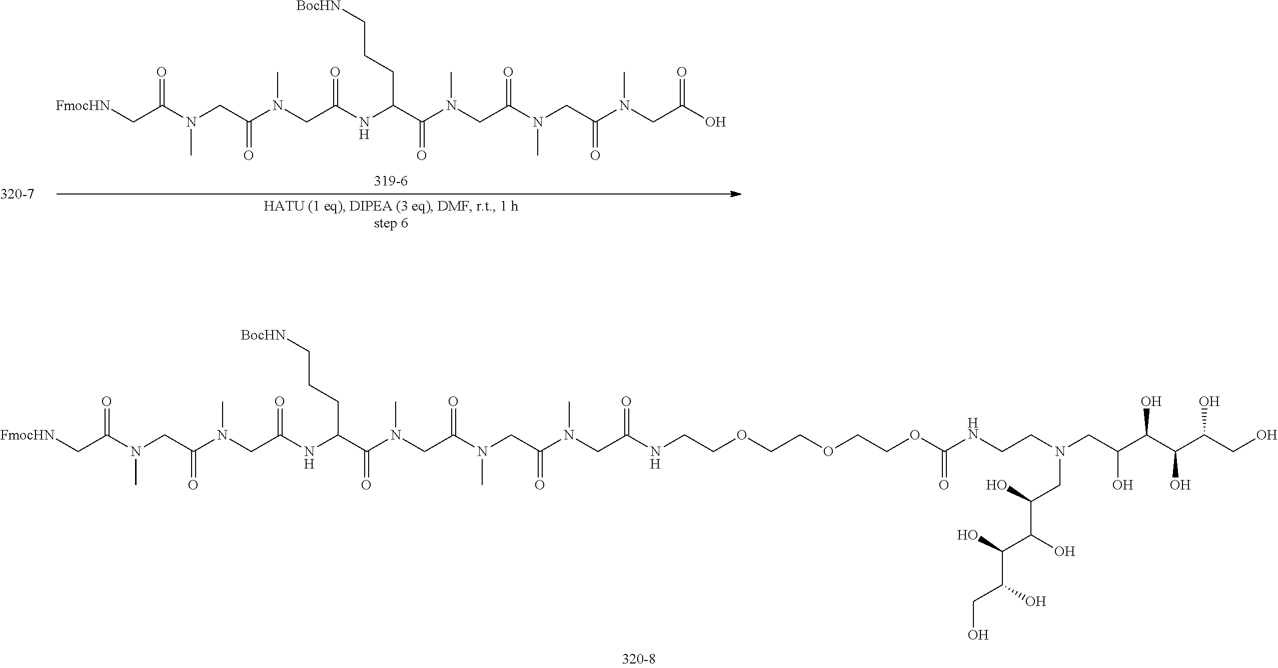

A solution of 320-7 (385 mg, 0.683 mmol), 319-6 (592 mg, 0.683 mmol) and HATU (260 mg, 0.684 mmol) in anhydrous DMF (6 mL) was stirred at room temperature for 5 min, then DIPEA (265 mg, 2.050 mmol) was added. The resulting solution was stirred for another 1 hr at r.t. until LCMS indicated complete reaction. The reaction solution was purified directly by reverse phase liquid chromatography to give 320-8 (413 mg, 0.292 mmol, 42.81%) as a white solid.

### Step 7

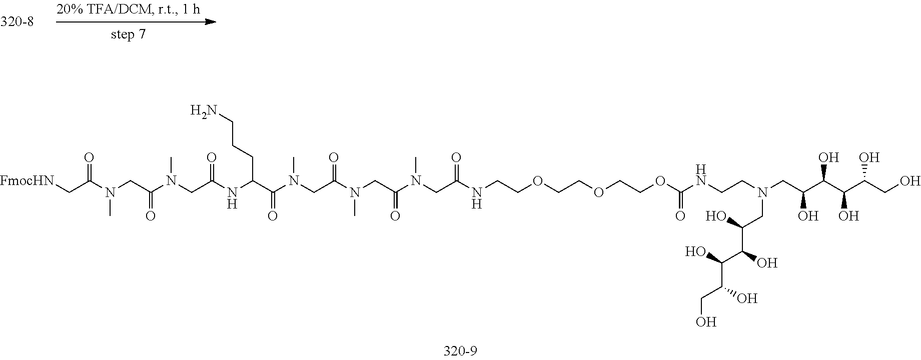

A solution of 320-8 (413 mg, 0.292 mmol) and TFA (2 mL) in anhydrous DCM (8 mL) was stirred at room temperature for 1 hr until LCMS indicated all starting amine was disappeared and desired product was detected. Then the solution was concentrated to dryness to 320-9 (370 mg, 0.262 mmol, 89.70%) as yellow oil, used as such in the next step.

### Step 8

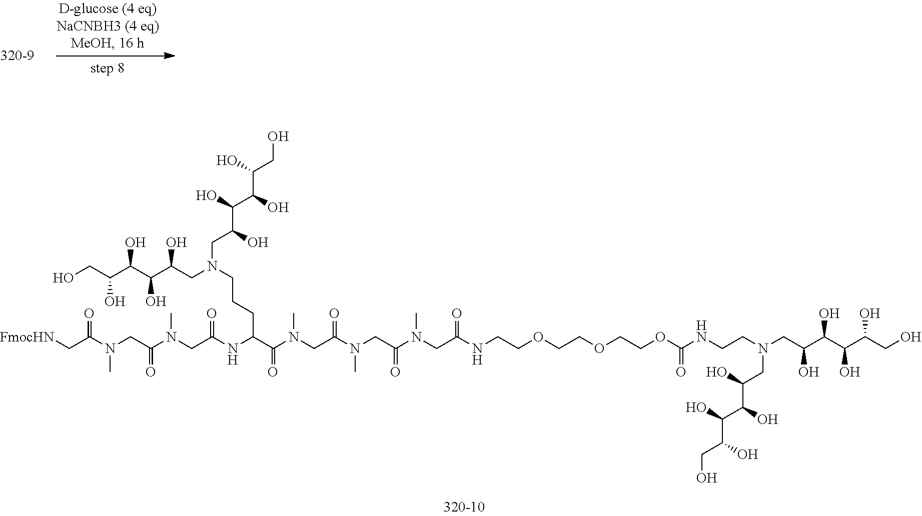

A solution of 320-9 (370 mg, 0.262 mmol) and D-Glucose (203 mg, 1.127 mmol) in anhydrous Methanol (40 mL) was heated at 50° C. for 30 min, then NaCNBH₃ (71 mg, 1.130 mmol) was added. The resulting solution was stirred for another 16 hr at 70° C. until indicated all starting amine was disappeared and the mass of desired product was detected. Then the reaction solution was concentrated and purified by reverse phase liquid chromatography to give 320-10 (210 mg, 0.128 mmol, 48.85%) as a white solid.

### Step 9

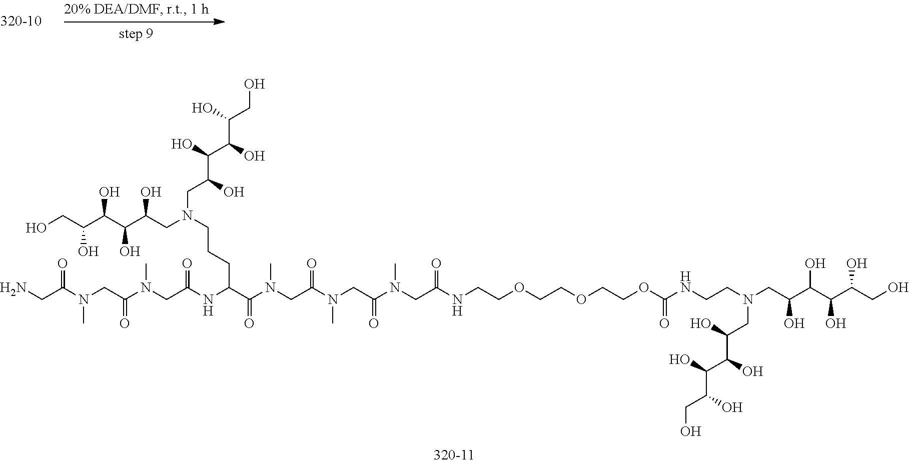 [SMILES: CN(CC(=O)NCCOCCOCCOC(=O)NCCN(C[C@H](O)C(O)[C@H](O)[C@H](O)CO)C[C@H](O)[C@@H](O)[C@H](O)[C@H](O)CO)C(=O)CN(C)C(=O)CN(C)C(=O)C(CCCN(C[C@H](O)[C@@H](O)[C@H](O)[C@H](O)CO)C[C@H](O)[C@@H](O)[C@H](O)[C@H](O)CO)NC(=O)CN(C)C(=O)CN(C)C(=O)CN]

A solution of 320-10 (210 mg, 0.128 mmol) and DEA (1 mL) in anhydrous DMF (4 mL) was stirred at room temperature for 1 hr until LCMS indicated all starting amine was disappeared and desired product was detected. Then the solution was concentrated to dryness to 320-11 (175 mg, 0.123 mmol, 96.38%) as colorless oil, used as such in the next step.

### Step 10

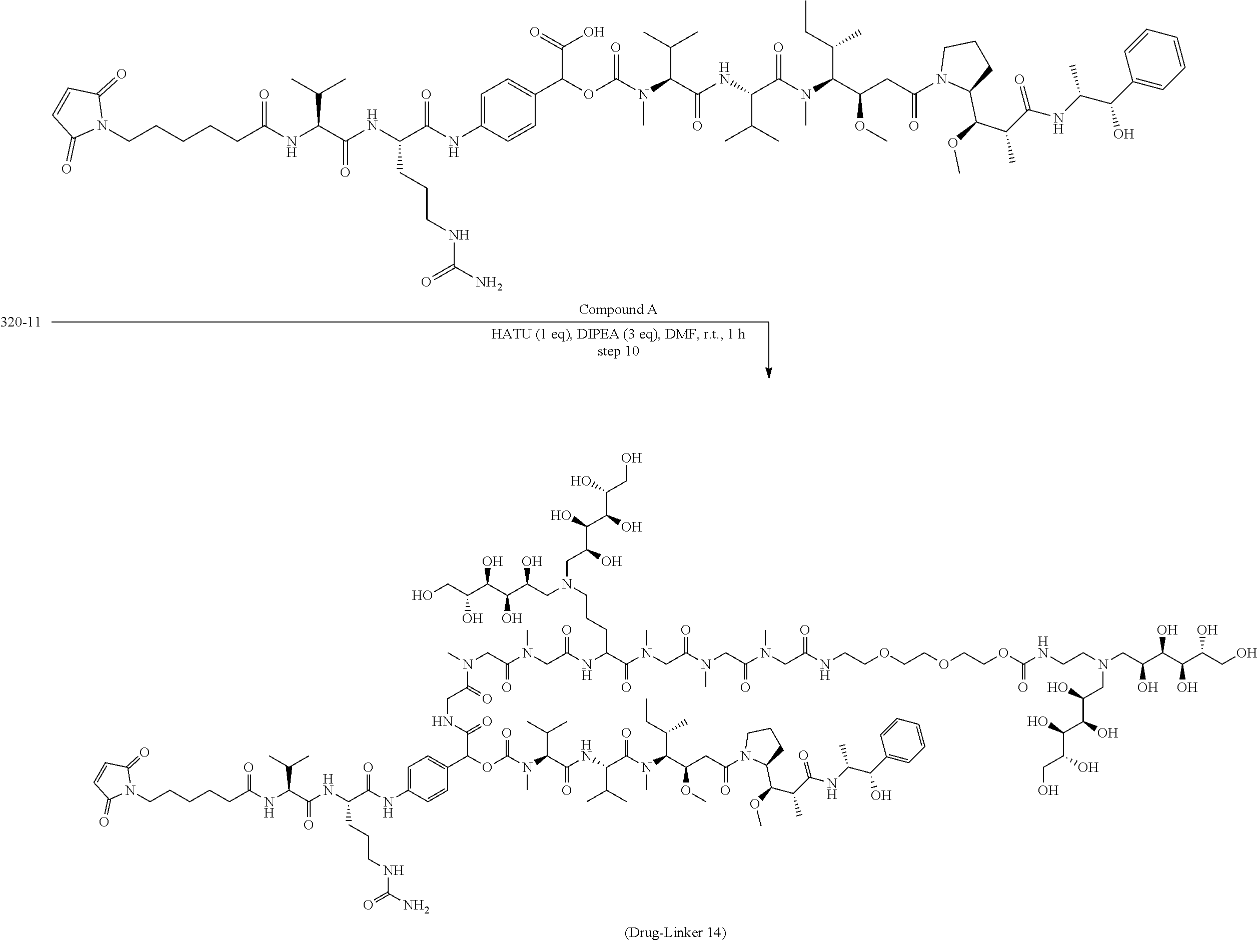

A solution of 320-11 (42 mg, 0.030 mmol), Compound A (40 mg, 0.029 mmol) and HATU (11 mg, 0.029 mmol) in anhydrous DMF (4 mL) was stirred at room temperature for 5 min, then DIPEA (11 mg, 0.085 mmol) was added. The resulting solution was stirred for another 1 hr at r.t. until LCMS indicated complete reaction. Then the reaction solution was purified by prep. HPLC to give Drug-Linker 14 (16 mg, 0.006 mmol, 19.32%) as a white solid. LCMS, m/z=1381.18 (M/2+H)⁺.

### Example 15: Preparation of Drug-Linker 15

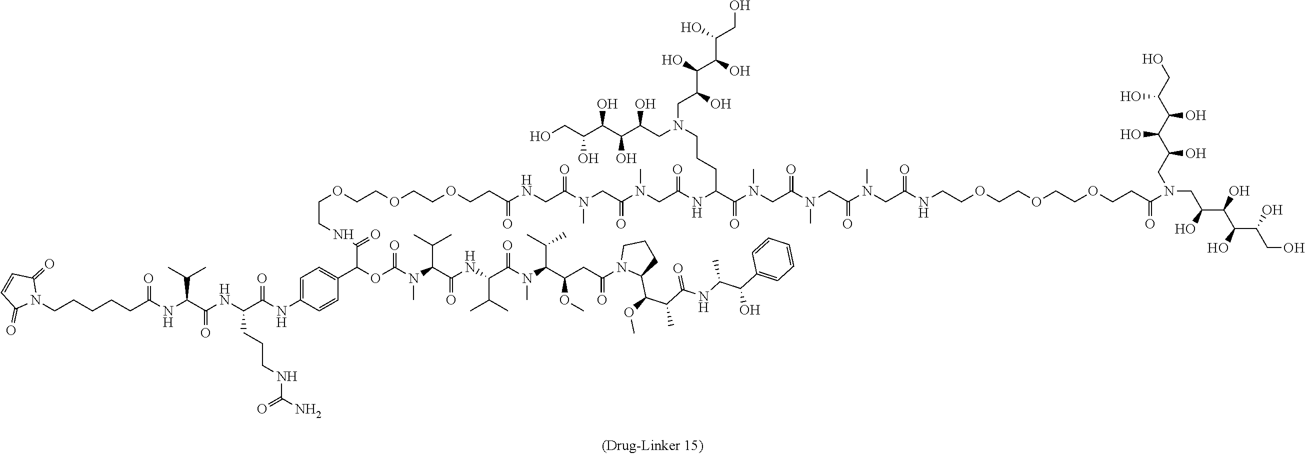

### Step 1

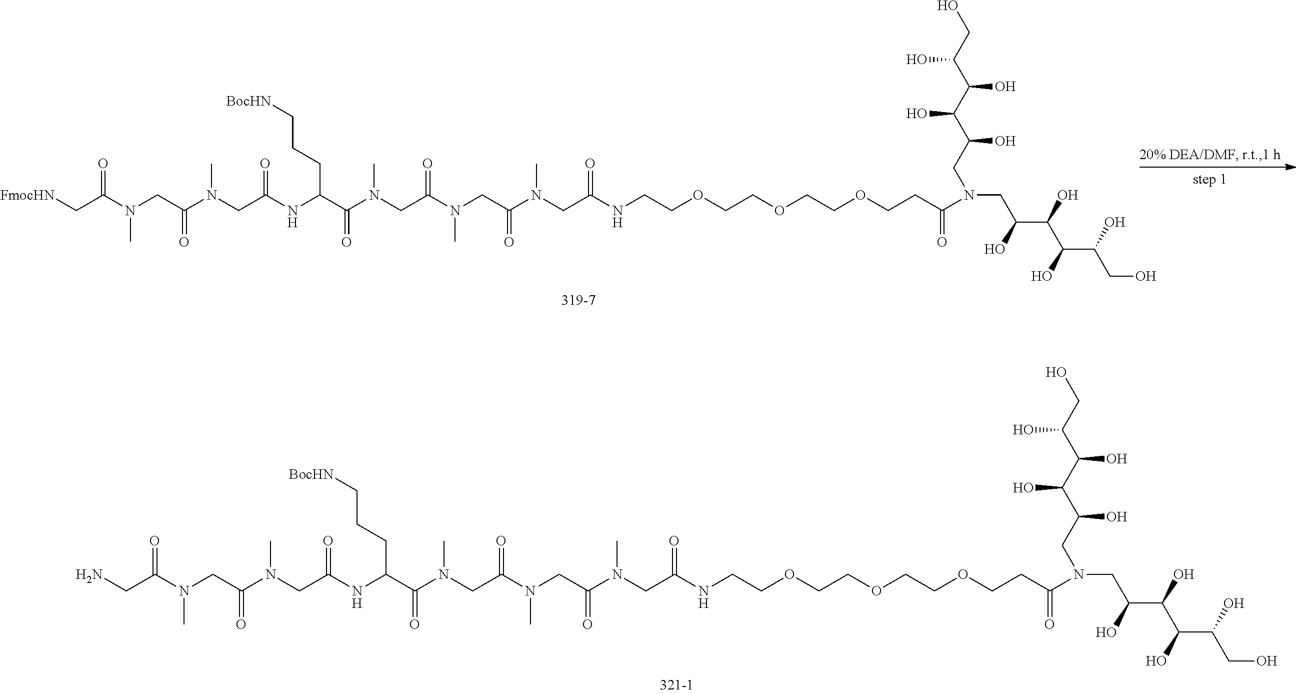

A solution of 319-7 (350 mg, 0.250 mmol) and DEA (1 mL) in anhydrous DMF (4 mL) was stirred at room temperature for 1 hour until LCMS indicated all starting amine was disappeared and desired product was detected. Then the solution was concentrated to dryness to 321-1 (280 mg, 0.238 mmol, 95.29%) as colorless oil, used as such in the next step.

### Step 2

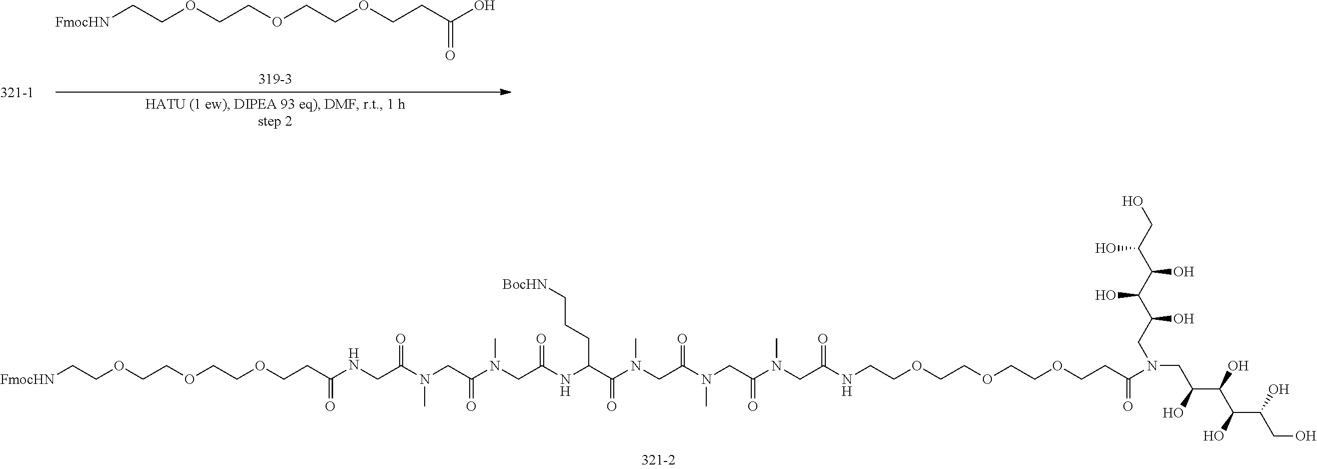

A solution of 321-1 (280 mg, 0.238 mmol), 319-3 (105 mg, 0.237 mmol) and HATU (90 mg, 0.237 mmol) in anhydrous DMF (10 mL) was stirred at room temperature for 5 min, then DIPEA (92 mg, 0.712 mmol) was added. The resulting solution was stirred for another 1 hour at r.t. until LCMS indicated complete reaction. The reaction solution was purified directly by reverse phase liquid chromatography to give 321-2 (265 mg, 0.166 mmol, 69.56%) as a white solid.

### Step 3

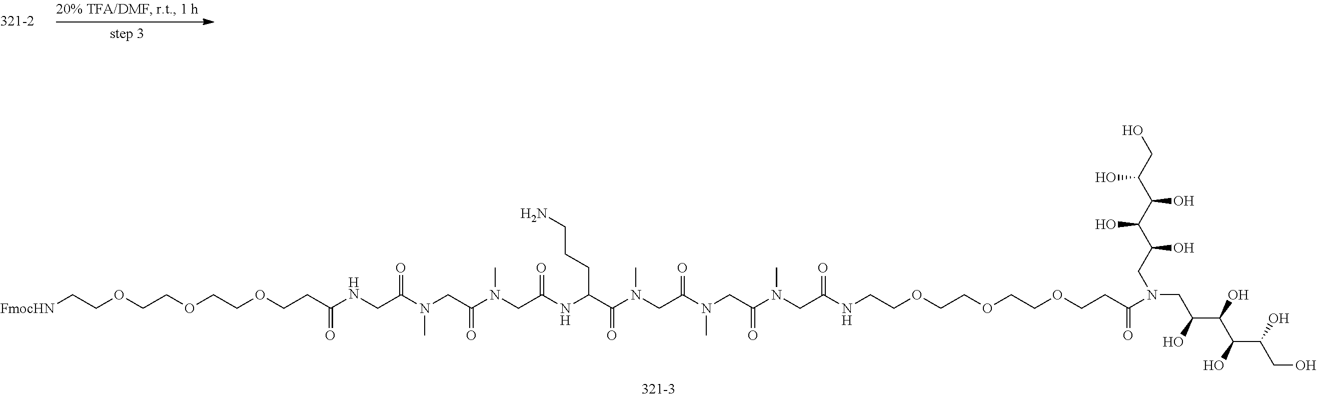

A solution of 321-2 (265 mg, 0.166 mmol) and TFA (1 mL) in anhydrous DCM (4 mL) was stirred at room temperature for 1 hr until LCMS indicated all starting amine was disappeared and desired product was detected. Then the solution was concentrated to dryness to 321-3 (243 mg, 0.162 mmol, 97.55%) as yellow oil, used as such in the next step.

### Step 4

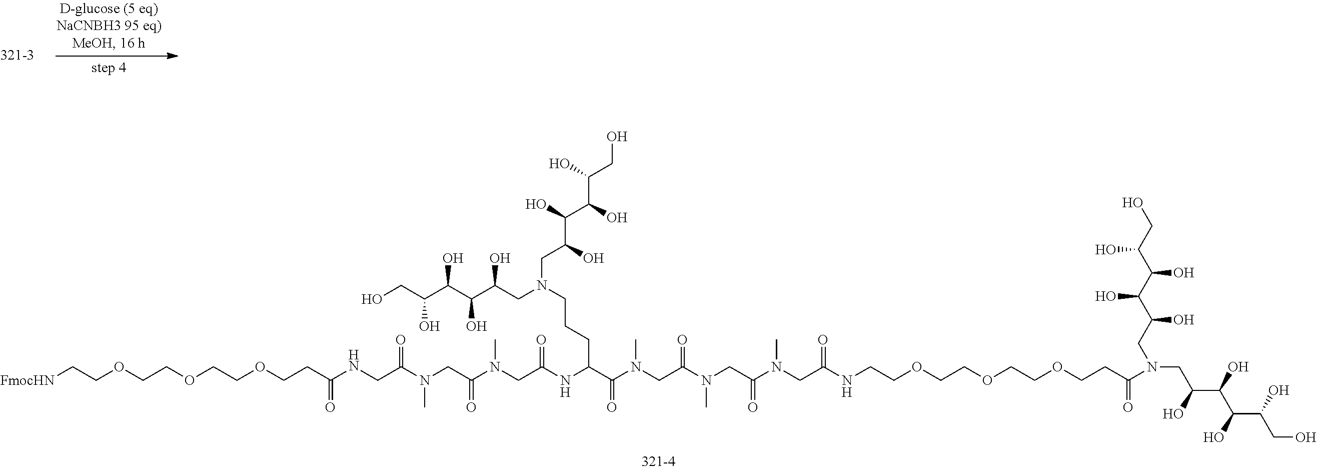

A solution of 321-3 (243 mg, 0.162 mmol) and D-Glucose (146 mg, 0.810 mmol) in anhydrous Methanol (50 mL) was heated at 50° C. for 30 min, then NaCNBH₃ (51 mg, 0.812 mmol) was added. The resulting solution was stirred for another 16 hr at 70° C. until indicated all starting amine was disappeared and the mass of desired product was detected. Then the reaction solution was concentrated and purified by reverse phase liquid chromatography to give 321-4 (155 mg, 0.085 mmol, 52.31%) as a white solid.

### Step 5

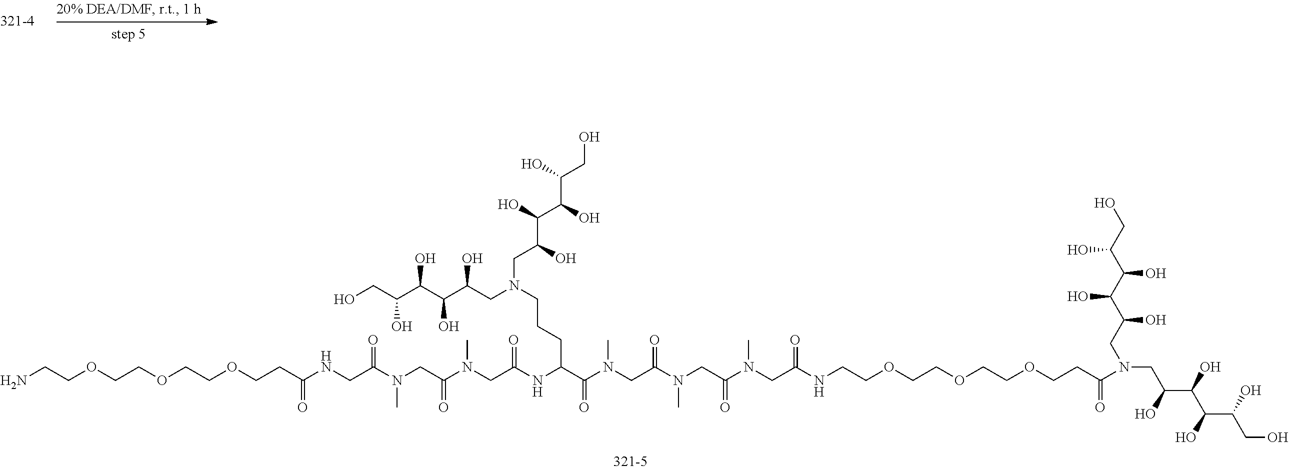 [SMILES: CN(CC(=O)NCCOCCOCCOCCC(=O)N(C[C@H](O)[C@@H](O)[C@H](O)[C@H](O)CO)C[C@H](O)[C@@H](O)[C@H](O)[C@H](O)CO)C(=O)CN(C)C(=O)CN(C)C(=O)C(CCCN(C[C@H](O)[C@@H](O)[C@H](O)[C@H](O)CO)C[C@H](O)[C@@H](O)[C@H](O)[C@H](O)CO)NC(=O)CN(C)C(=O)CN(C)C(=O)CNC(=O)CCOCCOCCOCCN]

A solution of 321-4 (155 mg, 0.085 mmol) and DEA (1 mL) in anhydrous DMF (4 mL) was stirred at room temperature for 1 hr until LCMS indicated all starting amine was disappeared and desired product was detected. Then the solution was concentrated to dryness to 321-5 (130 mg, 0.081 mmol, 95.19%) as colorless oil, used as such in the next step.

### Step 6

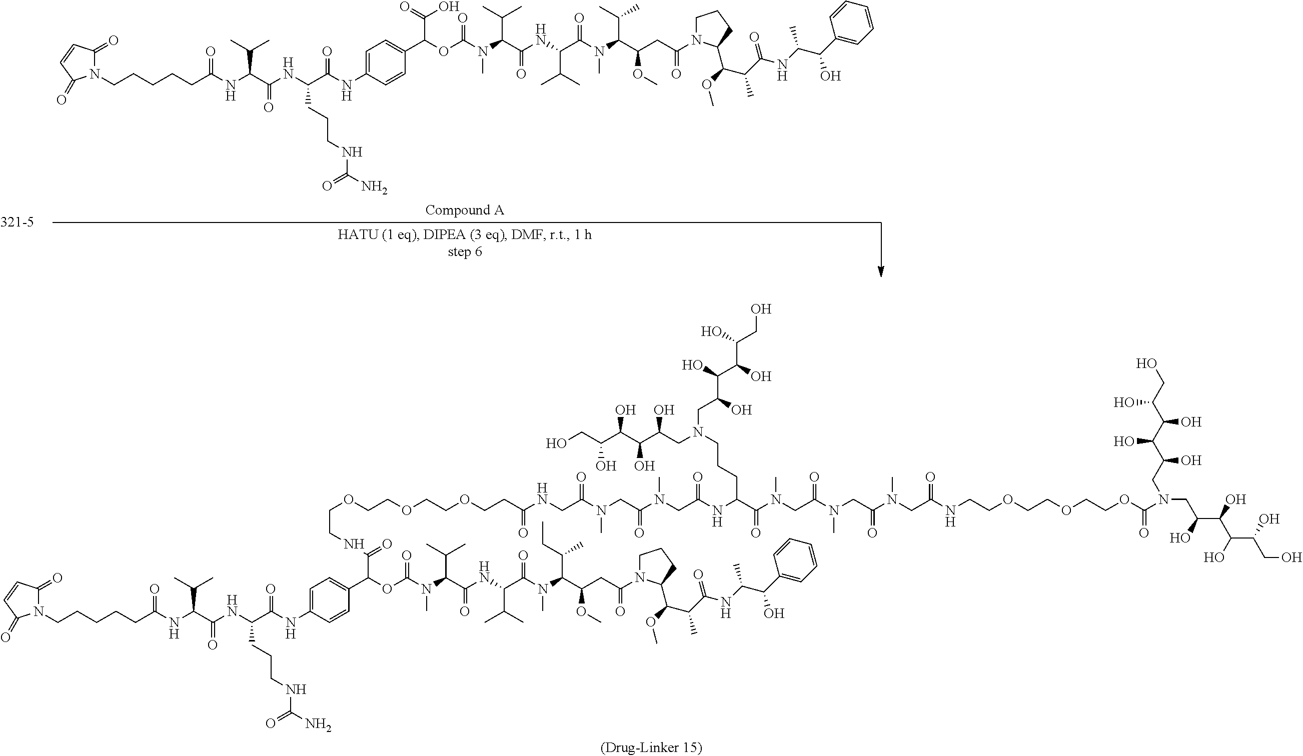

A solution of 321-5 (130 mg, 0.081 mmol), Compound A (110 mg, 0.081 mmol) and HATU (31 mg, 0.081 mmol) in anhydrous DMF (4 mL) was stirred at room temperature for 5 min, then DIPEA (31 mg, 0.240 mmol) was added. The resulting solution was stirred for another 1 hr at r.t. until LCMS indicated complete reaction. Then the reaction solution was purified by Prep. HPLC to give Drug-Linker 15 (24 mg, 0.008 mmol, 9.88%) as a white solid. LCMS, m/z M 984.06 (M/3+H)⁺.

### Example 16: Preparation of Drug-Linker 16

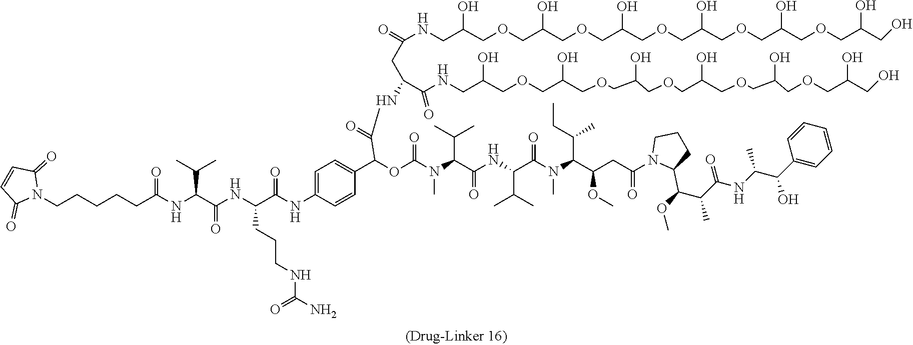 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@H](CC(=O)NCC(O)COCC(O)COCC(O)COCC(O)COCC(O)COCC(O)CO)C(=O)NCC(O)COCC(O)COCC(O)COCC(O)COCC(O)COCC(O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

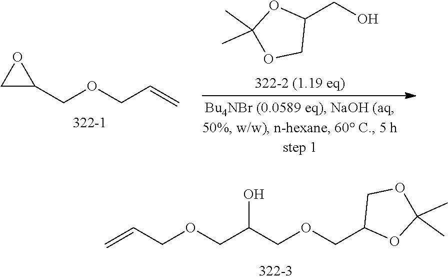

A solution of 322-2 (12.5 g, 94.64 mmol) and Bu₄NBr (1.51 g, 4.684 mmol) in mixture solvent of n-hexane (62.6 mL) and NaOH (aq, 50% w/w, 62.5 mL) was stirred at 60° C. A solution of 322-1 (9.07 g, 79.53 mmol) in n-hexane (12.5 mL) was dropped to the reaction mixture, and the resulting mixture was stirred at 60° C. for 5 hrs until most of 322-1 was consumed by TLC. The mixture was cooled down to r.t. and diluted with water (100 mL), then it was extracted with MTBE (200 mL×2). The organic parts were combined, dried over anhydrous Na₂SO₄, filtered and concentrated to dryness under reduced pressure. The crude product was purified with column chromatography (silica, 0-50% Ethyl acetate in petroleum ether) affording 322-3 as a colorless oil 20.68 g. ¹H NMR (400 MHz, CDCl₃) δ 5.93-5.80 (m, 1H), 5.30-5.20 (m, 1H), 5.19-5.12 (m, 1H), 4.30-4.15 (m, 1H), 4.05-3.91 (m, 4H), 3.73-3.66 (m, 1H), 3.58-3.43 (m, 6H), 2.74 (s, 1H), 1.39 (d, J=3.6 Hz, 3H), 1.33 (d, J=2.8 Hz, 3H).

### Step 2

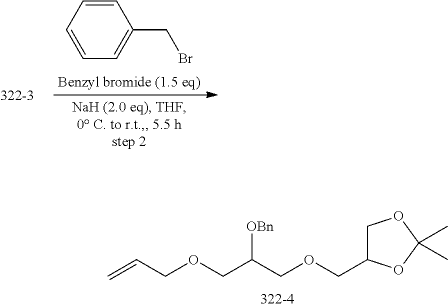 [SMILES: BrCc1ccccc1 ; C=CCOCC(COCC1COC(C)(C)O1)OCc1ccccc1]

To a mixture of NaH (5.58 g, 139.59 mmol) in anhydrous THF (340 mL) cooled in an ice bath was added a solution of 322-3 (17.18 g, 69.79 mmol) in anhydrous THF (85 mL) dropwise, then it was stirred at this temperature for 20 mins. Benzyl bromide (17.79 g, 104.69 mmol) was added dropwise, then the resulting mixture was allowed to warm to r.t. and stirred for 5 hrs until 322-3 was consumed by TLC. The reaction was quenched with saturated ammonium (150 mL) and extracted with ethyl acetate (200 mL×2). The organic phase was combined, dried over anhydrous Na₂SO₄, filtered and concentrated to dryness under reduced pressure to give the crude product, which was purified with column chromatography (silica, 0-40% ethyl acetate in petroleum ether) affording 322-4 (14.7 g, 43.73 mmol, 62.7%) as a colorless oil. Purity=90%-95%. 1H NMR (400 MHz, CDCl₃) δ 7.41-7.22 (m, 5H), 5.97-5.81 (m, 1H), 5.27 (dt, J=17.2, 1.8 Hz, 1H), 5.17 (dq, J=10.5, 1.5 Hz, 1H), 4.75-4.62 (m, 2H), 4.24 (t, J=6.0 Hz, 1H), 4.07-3.95 (m, 3H), 3.79-3.70 (m, 2H), 3.68-3.59 (m, 2H), 3.58-3.52 (m, 3H), 3.51-3.45 (m, 1H), 1.41 (s, 3H), 1.35 (s, 3H).

### Step 3

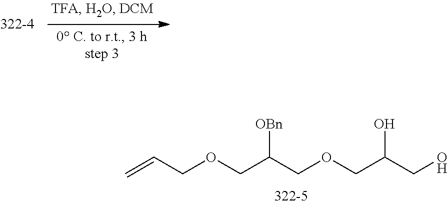 [SMILES: C=CCOCC(COCC(O)CO)OCc1ccccc1]

step 3

A solution of 322-4 (3.5 g, 10.411 mmol) in DCM (62.3 mL) and H₂O (0.78 mL) was stirred in an ice bath, then trifluoroacetic acid (1.56 mL) was added. The resulting solution was stirred for 3 hrs at this temperature until 322-4 was consumed by TLC. The reaction was quenched with saturated NaHCO₃(aq) and then diluted with water (20 mL). The mixture was extracted with DCM (30 mL×2) and the organic phase was combined, dried over anhydrous Na₂SO₄, filtered and concentrated to dryness under reduced pressure to give the crude product, which was purified with column chromatography (silica, 0-60% Ethyl acetate in petroleum ether) affording 322-5 (2.815 g, 9.505 mmol, 91.3%) as a colorless oil. Purity=90%-95%. 1H NMR (400 MHz, CDCl₃) δ 7.40-7.26 (m, 5H), 5.95-5.81 (m, 1H), 5.31-5.23 (m, 1H), 5.21-5.14 (m, 1H), 4.73-4.59 (m, 2H), 3.99 (dt, J=5.6, 1.5 Hz, 2H), 3.85-3.78 (m, 1H), 3.76-3.71 (m, 1H), 3.66-3.49 (m, 8H), 2.55 (s, 2H).

### Step 4

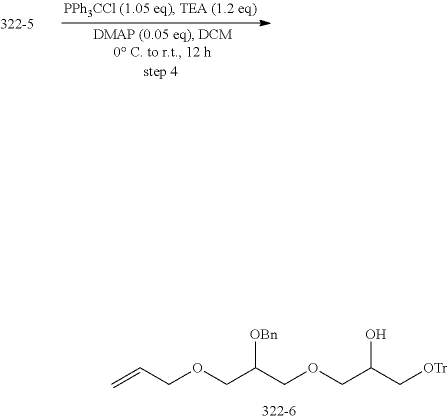

A solution of 322-5 (8.17 g, 27.586 mmol), TEA (3.349 g, 33.103 mmol) and DMAP (168.5 mg, 1.379 mmol) in DCM (40.8 mL) was stirred in an ice bath, then a solution of Ph₃CCl (8.075 g, 28.966 mmol) in DCM (40.8 mL) was added dropwise. The resulting mixture was gradually warmed to room temperature and stirred at ambient temperature for 12 hrs until most of 322-5 was consumed by TLC. The mixture was diluted with DCM (20 mL), washed with water (20 mL×2), dried over anhydrous Na₂SO₄, filtered and concentrated to dryness under reduced pressure to give the crude product, which was purified with column chromatography (silica, 0-40% Ethyl acetate in petroleum ether) affording 322-6 (13.86 g, 25.749 mmol, 93.3%) as a pale yellow oil. Purity=90%-95%. ¹H NMR (400 MHz, CDCl₃) δ 7.47-7.36 (m, 6H), 7.32-7.20 (m, 14H), 5.94-5.90 (m, 1H), 5.29-5.21 (m, 1H), 5.16 (dd, J=10.4, 1.6 Hz, 1H), 4.69-4.57 (m, 2H), 4.0-3.86 (m, 3H), 3.73-3.67 (m, 1H), 3.63-3.55 (m, 3H), 3.55-3.48 (m, 3H), 3.24-3.12 (m, 2H), 2.61 (s, 1H).

### Step 5

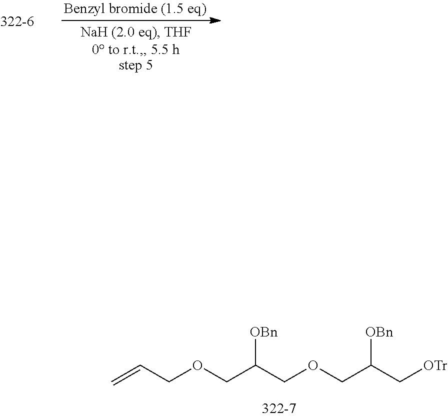

A mixture of NaH (2.06 g, 51.498 mmol) in anhydrous THF (90 mL) was stirred in an ice bath, then a solution of 322-6 (13.86 g, 25.749 mmol) in anhydrous THF (45 mL) was added dropwise. The reaction mixture was stirred at this temperature for 20 mins, then benzyl bromide (6.56 g, 38.62 mmol) was added. The resulting mixture was allowed to warm to r.t. and stirred for 5 hrs until 322-6 was consumed by TLC. The reaction was quenched with saturated ammonium (50 mL) and extracted with (ethyl acetate 60 mL×2). The organic phase was combined, dried over anhydrous Na₂SO₄, filtered and concentrated to dryness under reduced pressure to give the crude product, which was purified with column chromatography (silica, 0-30% ethyl acetate in petroleum ether) affording 322-7 (15.67 g, 24.940 mmol, 96.86%) as a colorless oil. Purity=90%-95%. ¹H NMR (400 MHz, CDCl₃) δ 7.51-7.40 (m, 6H), 7.35-7.20 (m, 19H), 5.94-5.80 (m, 1H), 5.29-5.21 (m, 1H), 5.17-5.10 (m, 1H), 4.65 (d, J=12.1 Hz, 4H), 3.99-3.87 (m, 2H), 3.79-3.65 (m, 2H), 3.65-3.59 (m, 2H), 3.58-3.53 (m, 2H), 3.52-3.46 (m, 2H), 3.23 (d, J=5.0 Hz, 2H).

### Step 6

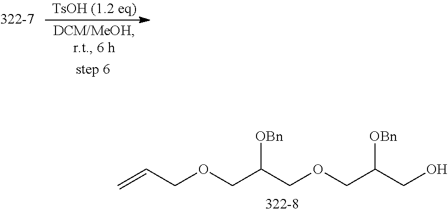 [SMILES: C=CCOCC(COCC(CO)OCc1ccccc1)OCc1ccccc1]

To a solution of 322-7 (15.67 g, 24.939 mmol) in a mixture solvent of DCM (62 mL) and MeOH (31 mL) was added TsOH·H₂O (5.7 g, 29.927 mmol), the resulting mixture was stirred for 6 hrs at room temperature until 322-7 was consumed by TLC. The reaction was quenched with saturated NaHCO₃ (aq.), diluted with water (30 mL), and then extracted with DCM (50 mL×3). The organic phase was combined, dried over anhydrous Na₂SO₄, filtered and concentrated to dryness under reduced pressure to give the crude product, which was purified with column chromatography (silica, 0-50% ethyl acetate in petroleum ether) affording 322-8 (9.09 g, 23.54 mmol, 94.37%) as a pale yellow oil. Purity=90%-95%. 1H NMR (400 MHz, CDCl₃) δ 7.48-7.15 (m, 10H), 5.98-5.80 (m, 1H), 5.26 (d, J=17.2 Hz, 1H), 5.17 (d, J=10.4 Hz, 1H), 4.75-4.53 (m, 4H), 3.99 (d, J=5.6 Hz, 2H), 3.78-3.70 (m, 2H), 3.68-3.48 (m, 8H).

### Step 7

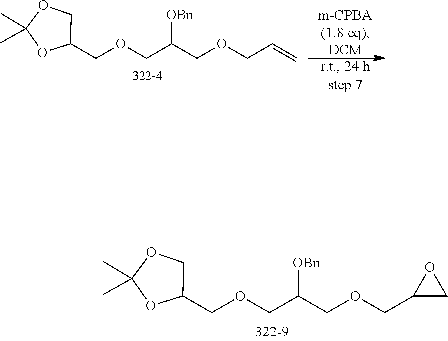

To a solution of 322-4 (14.0 g, 41.555 mmol) in DCM (36 mL) was added a solution of m-CPBA (12.91 g, 74.799 mmol) in DCM (103 mL) dropwise at room temperature. The resulting mixture was stirred at this temperature for 24 hours until 322-4 was consumed, and the reaction was quenched with saturated Na₂S₂O₃ (aq., 50 mL) and NaHCO₃ (aq, 50 mL). The reaction mixture was stirred for 30 mins, and then diluted with DCM (150 mL). The organic phase was washed with a mixture solution (60 mL×3) of saturated Na₂S₂O₃ and NaHCO₃ (aq, 1:1, V/V), dried over anhydrous Na₂SO₄, filtered and concentrated to dryness under reduced pressure to give the crude product, which was purified with column chromatography (silica, 0-60% Ethyl acetate in petroleum ether) affording 322-9 (13.79 g, 39.16 mmol, 94.37%) as a pale yellow oil. Purity=90%-95%. ¹H NMR (400 MHz, CDCl₃) δ 7.45-7.16 (m, 5H), 4.69 (s, 2H), 4.25 (t, J=5.6 Hz, 1H), 4.04 (t, J=7.2 Hz, 1H), 3.86-3.69 (m, 3H), 3.68-3.36 (m, 7H), 3.19-3.08 (m, 1H), 2.83-2.76 (m, 1H), 2.63-2.53 (m, 1H), 1.42 (s, 3H), 1.37 (s, 3H).

### Step 8

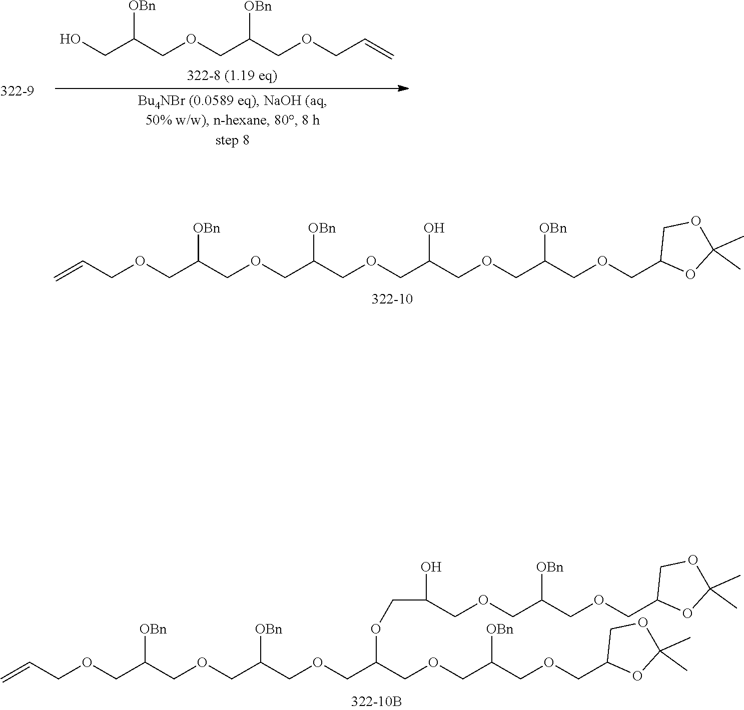

A solution of 322-8 (8.429 g, 23.934 mmol) and Bu₄NBr (454.4 mg, 1.409 mmol) in mixture solvent of n-hexane (30 mL) and NaOH (aq, 50% w/w, 30 mL) was stirred at 80° C. A mixture of 322-9 (11.0 g, 28.482 mmol) in n-hexane (6 mL) was dropped to the reaction mixture, and the resulting mixture was stirred at 80° C. for 8 hrs until most of 322-9 was consumed by TLC. The mixture was cooled down to r.t. and diluted with water (50 mL), then it was extracted with MTBE (100 mL×3). The organic parts were combined, dried over anhydrous Na₂SO₄, filtered and concentrated to dryness under reduced pressure. The crude product was purified with column chromatography (silica, 0-100% ethyl acetate in petroleum ether) affording 322-10 (8.0 g, 10.83 mmol, 45.3%) as a pale yellow oil and 322-10B (2.1 g, 1.925 mmol, 8.0%) as a pale yellow oil. ¹H NMR (400 MHz, CDCl₃) δ 7.38-7.15 (m, 15H), 5.96-5.73 (m, 1H), 5.31-5.06 (m, 2H), 4.76-4.51 (m, 6H), 4.29-4.13 (m, 1H), 4.07-3.86 (m, 4H), 3.76-3.37 (m, 22H), 2.52 (s, 1H), 1.56-1.05 (m, 6H). 1H NMR (400 MHz, CDCl₃) δ 7.42-7.27 (m, 20H), 5.96-5.82 (m, 1H), 5.26 (dd, J=17.2, 1.8 Hz, 1H), 5.16 (dd, J=10.5, 1.9 Hz, 1H), 4.69-4.61 (m, 8H), 4.27-4.19 (m, 2H), 4.05-3.97 (m, 4H), 3.90-3.80 (m, 2H), 3.76-3.66 (m, 8H), 3.61-3.48 (m, 26H), 3.09 (s, 1H), 1.58-1.16 (m, 12H).

### Step 9

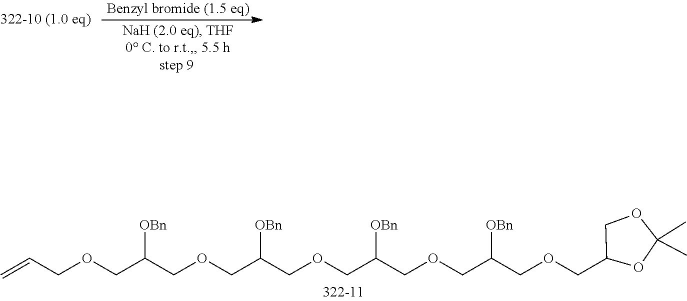 [SMILES: C=CCOCC(COCC(COCC(COCC(COCC1COC(C)(C)O1)OCc1ccccc1)OCc1ccccc1)OCc1ccccc1)OCc1ccccc1]

A mixture of NaH (867 mg, 21.668 mmol) in anhydrous THF (36 mL) was stirred in an ice bath, then a solution of 322-10 (8.0 g, 10.834 mmol) in anhydrous THF (20 mL) was added dropwise. The reaction mixture was stirred at this temperature for 20 mins, then benzyl bromide (2.76 g, 16.251 mmol) was added. The resulting mixture was allowed to warm to r.t. and stirred for 5 hrs until 322-10 was consumed by TLC. The reaction was quenched with saturated ammonium (25 mL) and extracted with ethyl acetate (40 mL×2). The organic phase was combined, dried over anhydrous Na₂SO₄, filtered and concentrated to dryness under reduced pressure to give the crude product, which was purified with column chromatography (silica, 0-60% Ethyl acetate in petroleum ether) affording 322-11 (8.45 g, 10.20 mmol, 94.1%) as a pale yellow oil. Purity=90%-95%. ¹H NMR (400 MHz, CDCl₃) δ 7.43-7.25 (m, 20H), 5.99-5.82 (m, 1H), 5.27 (dd, J=17.2, 1.8 Hz, 1H), 5.17 (dd, J=10.4, 1.7 Hz, 1H), 4.77-4.60 (m, 8H), 4.30-4.18 (m, 1H), 4.06-3.96 (m, 3H), 3.81-3.70 (m, 5H), 3.63-3.47 (m, 18H), 1.47-1.21 (m, 6H).

### Step 10

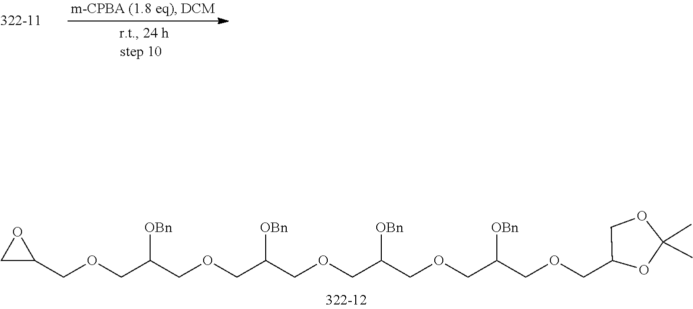 [SMILES: CC1(C)OCC(COCC(COCC(COCC(COCC(COCC2CO2)OCc2ccccc2)OCc2ccccc2)OCc2ccccc2)OCc2ccccc2)O1]

To a solution of 322-11 (8.45 g, 10.20 mmol) in DCM (12.8 mL) was added a solution of m-CPBA (3.16 g, 18.36 mmol) in DCM (25.6 mL) dropwise at room temperature. The resulting mixture was stirred at this temperature for 24 hours until 322-11 was consumed, and the reaction was quenched with saturated Na₂S₂O₃ (aq, 15 mL) and NaHCO₃ (aq, 15 mL). The reaction mixture was stirred for 30 mins, and then diluted with DCM (120 mL). The organic phase was washed with a mixture solution (40 mL×3) of saturated Na₂S₂O₃ and NaHCO₃ (aq, 1:1, V/V), dried over anhydrous Na₂SO₄, filtered and concentrated to dryness under reduced pressure to give the crude product, which was purified with column chromatography (silica, 0-60% ethyl acetate in petroleum ether) affording 322-12 (6.858 g, 8.12 mmol, 79.6%) as a pale yellow oil. Purity=90%-95%. ¹H NMR (400 MHz, CDCl₃) δ 7.53-7.25 (m, 20H), 4.68 (s, 8H), 4.28-4.20 (m, 1H), 4.06-4.00 (m, 1H), 3.80-3.69 (m, 6H), 3.66-3.50 (m, 16H), 3.49-3.44 (m, 1H), 3.41-3.35 (m, 1H), 3.16-3.08 (m, 1H), 2.76 (t, J=4.6 Hz, 1H), 2.63-2.52 (m, 1H), 1.54-1.24 (m, 6H).

### Step 11

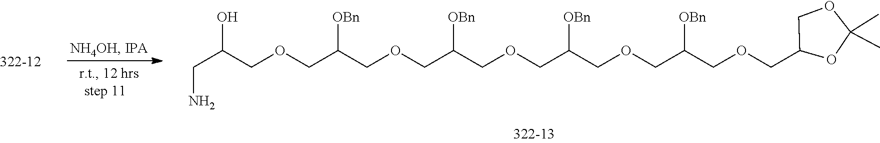 [SMILES: CC1(C)OCC(COCC(COCC(COCC(COCC(COCC(O)CN)OCc2ccccc2)OCc2ccccc2)OCc2ccccc2)OCc2ccccc2)O1]

To a solution of 322-12 (1.22 g, 1.445 mmol) in isopropyl alcohol (120 mL) was added ammonia (120 mL) dropwise at room temperature, and the resulting mixture was stirred at this temperature for 12 hours until 322-12 was consumed. The reaction mixture was concentrated to dryness under reduced pressure to afford 322-13 (1.28 g, 1.48 mmol, 100%) as a colorless oil, which was used in the next step without further purification. Purity=90%-95%.

### Step 12

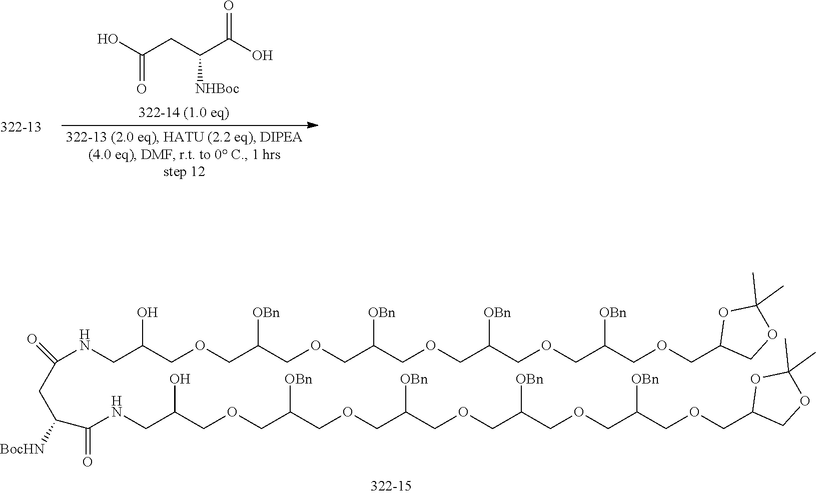 [SMILES: CC(C)(C)OC(=O)N[C@H](CC(=O)O)C(=O)O ; CC(C)(C)OC(=O)N[C@H](CC(=O)NCC(O)COCC(COCC(COCC(COCC(COCC1COC(C)(C)O1)OCc1ccccc1)OCc1ccccc1)OCc1ccccc1)OCc1ccccc1)C(=O)NCC(O)COCC(COCC(COCC(COCC(COCC1COC(C)(C)O1)OCc1ccccc1)OCc1ccccc1)OCc1ccccc1)OCc1ccccc1]

A solution of 322-14 (81.2 mg, 0.348 mmol) and HATU (291 mg, 0.766 mmol) in anhydrous DMF (5 mL) was stirred at room temperature for 15 mins, then it was stirred in an ice bath. A solution of 322-13 (600 mg, 0.696 mmol) in anhydrous DMF (3 mL) was added dropwise, followed by DIPEA (180 mg, 1.392 mmol). The resulting mixture was stirred in the ice bath for 1 h until most of 322-14 was consumed, and then diluted with ethyl acetate (60 mL). The organic phase was washed with H₂O (20 mL×3), dried over anhydrous Na₂SO₄, filtered and concentrated to dryness under reduced pressure to give the crude product, which was purified with column chromatography (silica, 0-100% ethyl acetate in petroleum ether) affording 322-15 (660 mg, 0.343 mmol, 98.7%) as a pale yellow oil. Purity=90%-95%. ¹H NMR (400 MHz, CDCl₃) δ 7.74-7.24 (m, 40H), 6.68-6.40 (m, 1H), 6.25-6.00 (m, 1H), 4.84-4.50 (m, 16H), 4.40 (s, 1H), 4.23 (t, J=6.0 Hz, 2H), 4.05-3.97 (m, 2H), 3.82-3.36 (m, 53H), 3.22-2.96 (m, 3H), 2.93-2.61 (m, 5H), 2.52-2.33 (m, 1H), 1.44-1.26 (m, 21H).

### Step 13

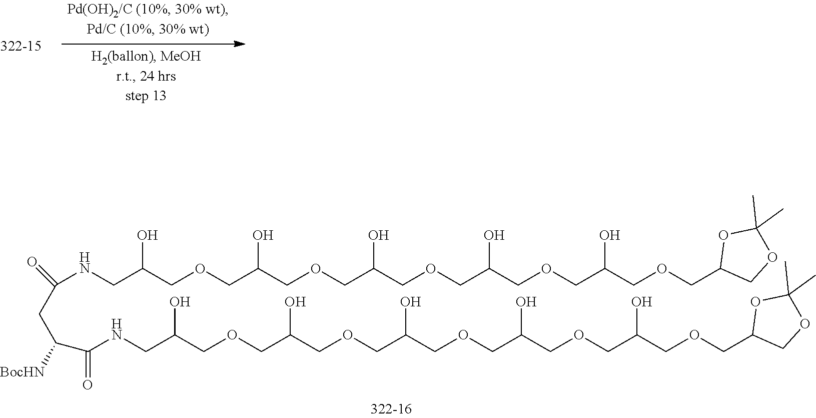 [SMILES: CC(C)(C)OC(=O)N[C@H](CC(=O)NCC(O)COCC(O)COCC(O)COCC(O)COCC(O)COCC1COC(C)(C)O1)C(=O)NCC(O)COCC(O)COCC(O)COCC(O)COCC(O)COCC1COC(C)(C)O1]

A mixture of 322-15 (160 mg, 0.0833 mmol), Pd(OH)₂/C (10%, 50 mg) and Pd/C (10%, 50 mg) in MeOH (10 mL) was stirred under hydrogen atmosphere (ballon) for 24 h at room temperature until 322-15 was completely converted into 322-16. The reaction mixture was filtered through a celite pad, and the filtrate was concentrated to dryness under reduced pressure to afford 322-16 (78.3 mg, 0.0652 mmol, 78.3%) as a colorless oil, which was used in the next step without further purification.

### Step 14

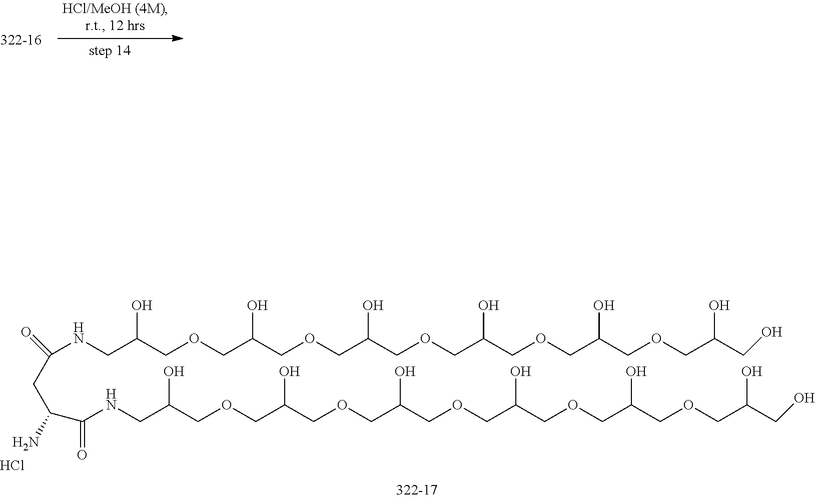 [SMILES: N[C@H](CC(=O)NCC(O)COCC(O)COCC(O)COCC(O)COCC(O)COCC(O)CO)C(=O)NCC(O)COCC(O)COCC(O)COCC(O)COCC(O)COCC(O)CO ; Cl]

A mixture of 322-16 (78.3 mg, 0.0652 mmol) and HCl/MeOH (4M, 4 mL) was stirred at room temperature for 12 hours until 322-16 was completely converted into 322-17. The reaction mixture was concentrated to dryness under reduced pressure to afford 322-17 (65.0 mg, 0.0616 mmol, 94.4%) as an off-white solid, which was used in the next step without further purification.

### Step 15

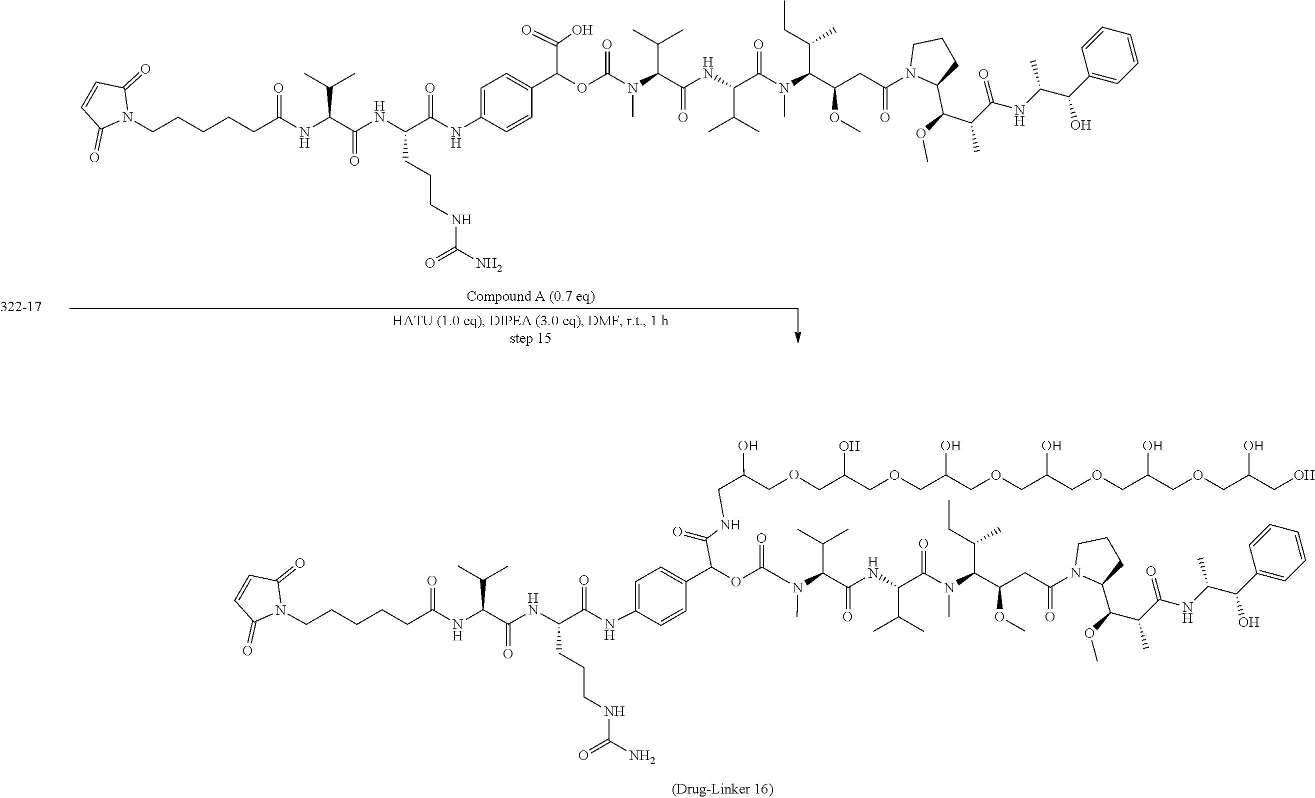

A solution of 322-17 (60 mg, 0.0568 mmol), Compound A (54 mg, 0.0397 mmol), HATU (21.6 mg, 0.0568 mmol) and DIPEA (22 mg, 0.170 mmol) in anhydrous DMF (4 mL) was stirred at room temperature for 1 h until Compound A was consumed. Then the reaction solution was purified by prep-HPLC to give Drug-Linker 16 (18.1 mg, 0.00766 mmol, 19.3%) as a white solid. LCMS, m/z=1182.02 (M/2+H)⁺, m/z=788.25 (M/3+H)⁺.

### Example 17: Preparation of Drug-Linker 17

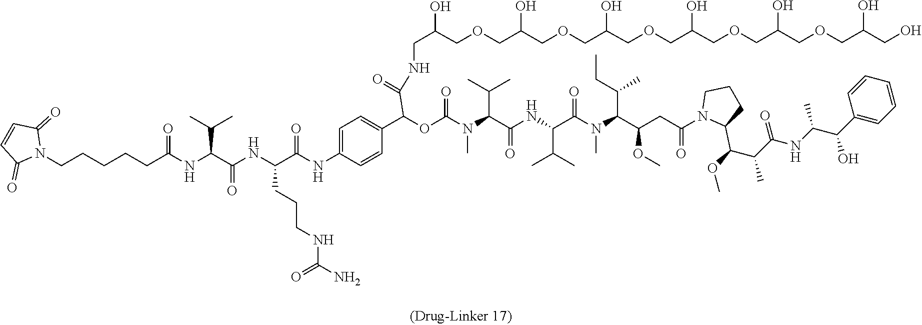 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCC(O)COCC(O)COCC(O)COCC(O)COCC(O)COCC(O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

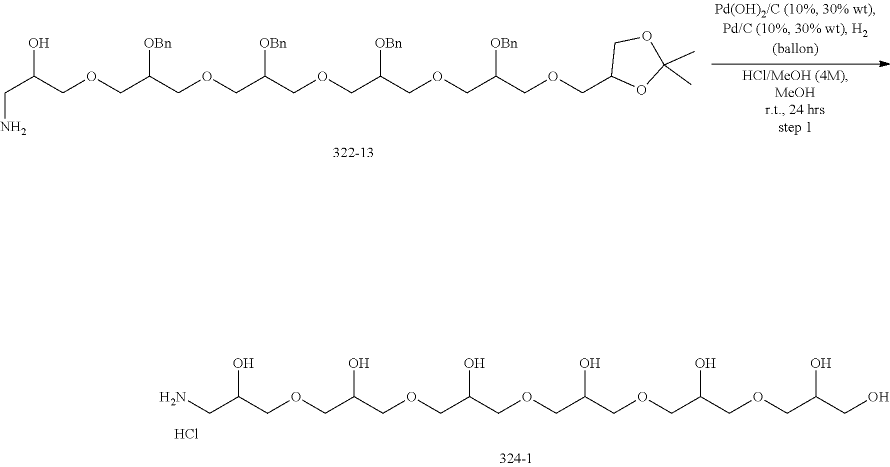 [SMILES: CC1(C)OCC(COCC(COCC(COCC(COCC(COCC(O)CN)OCc2ccccc2)OCc2ccccc2)OCc2ccccc2)OCc2ccccc2)O1 ; NCC(O)COCC(O)COCC(O)COCC(O)COCC(O)COCC(O)CO ; Cl]

A mixture of 322-13 (160 mg, 0.186 mmol), Pd(OH)₂/C (10%, 50 mg) and Pd/C (10%, 50 mg) in a mixed solvent of MeOH (10 mL) and HCl/MeOH (4M, 3 mL) was stirred under hydrogen atmosphere (ballon) for 24 h at room temperature until 322-13 was completely converted into 324-1. The reaction mixture was filtered through a celite pad, and the filtrate was concentrated to dryness under reduced pressure to afford 324-1 (92.3 mg, 0.186 mmol, 100%) as an off-white solid, which was used in the next step without further purification.

### Step 2

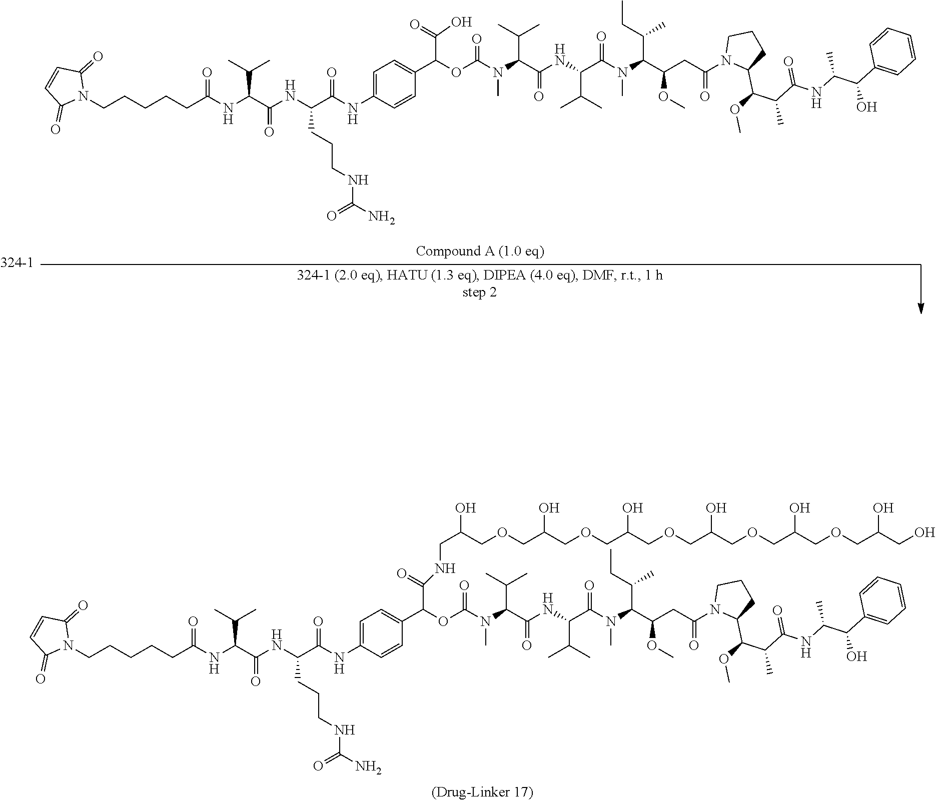

A solution of 324-1 (44 mg, 0.0885 mmol), Compound A (60 mg, 0.0441 mmol), HATU (21.8 mg, 0.0573 mmol) and DIPEA (22.8 mg, 0.176 mmol) in anhydrous DMF (4 mL) was stirred at room temperature for 1 h until Compound A was consumed. Then the reaction solution was purified by prep-HPLC to give Drug-Linker 17 (28.0 mg, 0.0155 mmol, 35.2%) as a white solid. LCMS, m/z=1804.11 (M+H)⁺, m/z=902.76 (M/2+H)⁺.

### Example 18: Preparation of Drug-Linker 18

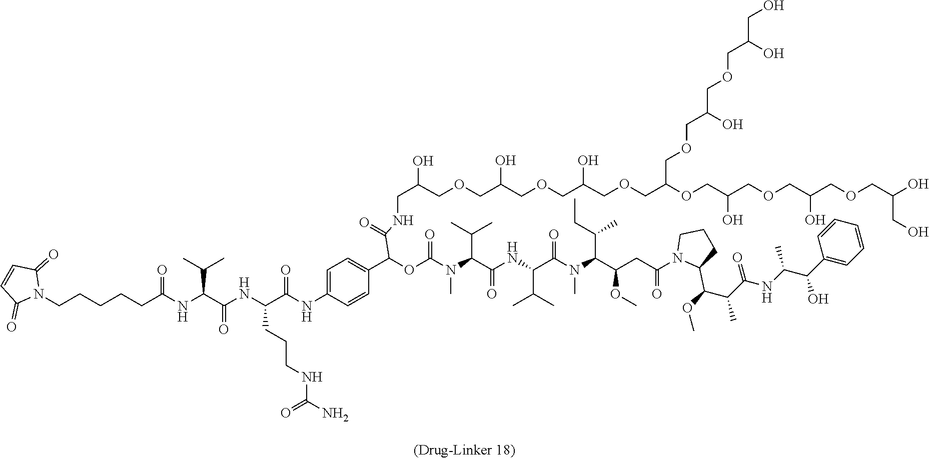 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCC(O)COCC(O)COCC(O)COCC(COCC(O)COCC(O)CO)OCC(O)COCC(O)COCC(O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

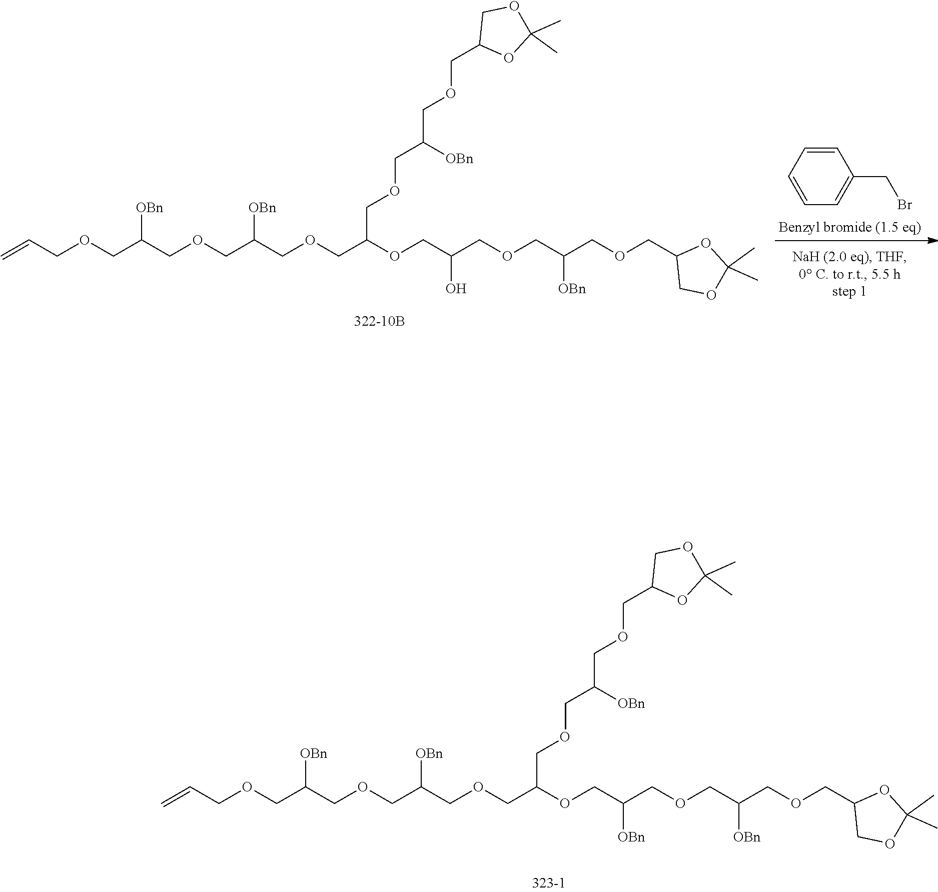

A mixture of NaH (205 mg, 5.13 mmol) in anhydrous THF (12.6 mL) was stirred in an ice bath, then a solution of 322-10B (2.79 g, 2.565 mmol) in anhydrous THF (7 mL) was added dropwise. The reaction mixture was stirred at this temperature for 20 mins, then benzyl bromide (654 g, 3.847 mmol) was added. The resulting mixture was allowed to warm to r.t. and stirred for 5 hrs until 322-10B was consumed by TLC. The reaction was quenched with saturated ammonium (15 mL) and extracted with ethyl acetate (30 mL×2). The organic phase was combined, dried over anhydrous Na₂SO₄, and concentrated to dryness under reduced pressure to give the crude product, which was purified with column chromatography (silica, 0-80% ethyl acetate in petroleum ether) affording 323-1 (2.772 g, 2.348 mmol, 91.8%) as a pale yellow oil. Purity=90%-95%.

### Step 2

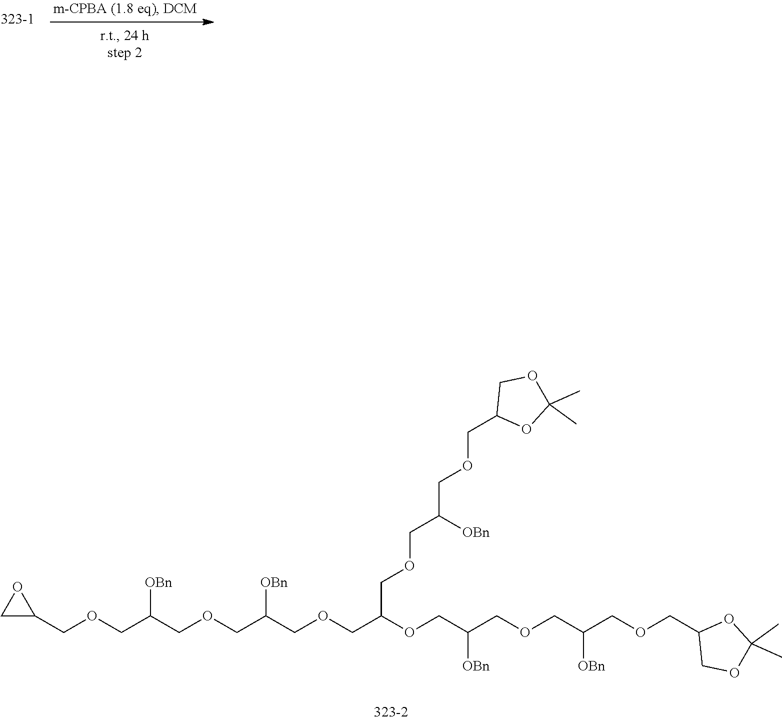 [SMILES: CC1(C)OCC(COCC(COCC(COCC(COCC(COCC2CO2)OCc2ccccc2)OCc2ccccc2)OCC(COCC(COCC2COC(C)(C)O2)OCc2ccccc2)OCc2ccccc2)OCc2ccccc2)O1]

To a solution of 323-1 (2.77 g, 2.346 mmol) in DCM (4.2 mL) was added a solution of m-CPBA (729 mg, 4.223 mmol) in DCM (8.4 mL) dropwise at room temperature. The resulting mixture was stirred at this temperature for 24 hours until 323-1 was consumed, and the reaction was quenched with saturated Na₂S₂O₃ (aq., 10 mL) and NaHCO₃ (aq, 10 mL). The reaction mixture was stirred for 30 mins, and then diluted with DCM (100 mL). The organic phase was washed with a mixture solution (30 mL×3) of saturated Na₂S₂O₃ and NaHCO₃ (aq, 1:1, V/V), dried over anhydrous Na₂SO₄, filtered and concentrated to dryness under reduced pressure to give the crude product, which was purified with column chromatography (silica, 0-100% ethyl acetate in petroleum ether) affording 323-2 (2.147 g, 1.79 mmol, 76.5%) as a pale yellow oil. Purity=90%-95%. ¹H NMR (400 MHz, CDCl₃) δ 7.50-7.20 (m, 25H), 4.65 (s, 10H), 4.22 (t, J=6.0 Hz, 2H), 4.06-3.98 (m, 2H), 3.82-3.30 (m, 38H), 3.15-3.03 (m, 1H), 2.82-2.70 (m, 1H), 2.62-2.51 (m, 1H), 1.46-1.28 (m, 12H).

### Step 3

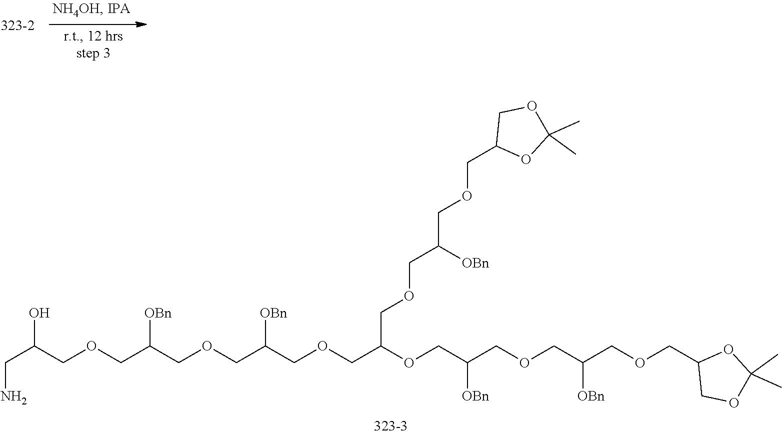 [SMILES: CC1(C)OCC(COCC(COCC(COCC(COCC(COCC(O)CN)OCc2ccccc2)OCc2ccccc2)OCC(COCC(COCC2COC(C)(C)O2)OCc2ccccc2)OCc2ccccc2)OCc2ccccc2)O1]

To a solution of 323-2 (450 mg, 0.376 mmol) in isopropyl alcohol (32 mL) was added ammonia (32 mL) dropwise at room temperature, and the resulting mixture was stirred at this temperature for 12 hours until 323-2 was consumed. The reaction mixture was concentrated to dryness under reduced pressure to afford 323-3 (456 mg, 1.48 mmol, 100%) as a colorless oil, which was used in the next step without further purification. Purity=90%-95%.

### Step 4

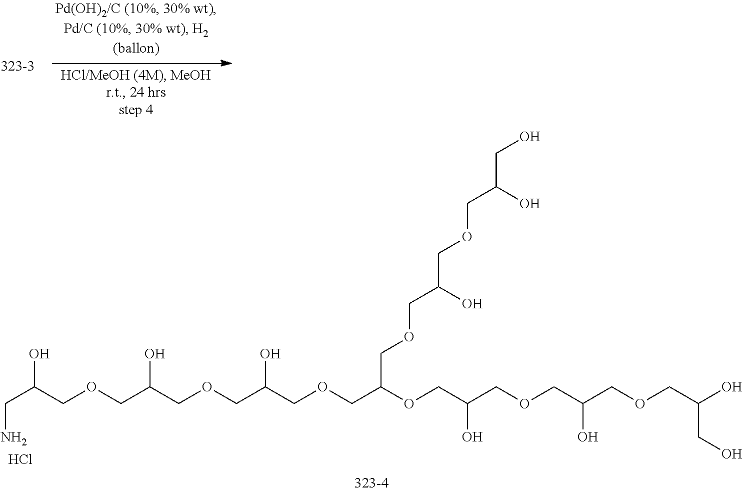 [SMILES: NCC(O)COCC(O)COCC(O)COCC(COCC(O)COCC(O)CO)OCC(O)COCC(O)COCC(O)CO ; Cl]

A mixture of 323-3 (250 mg, 0.206 mmol), Pd(OH)₂/C (10%, 75 mg) and Pd/C (10%, 75 mg) in a mixed solvent of MeOH (16 mL) and HCl/MeOH (4M, 4 mL) was stirred under hydrogen atmosphere (ballon) for 24 h at room temperature until 323-3 was completely converted into 323-4. The reaction mixture was filtered through a celite pad, and the filtered was concentrated to dryness under reduced pressure to afford 323-4 (149.7 mg, 0.208 mmol, 100%) as an off-white solid, which was used in the next step without further purification.

### Step 5

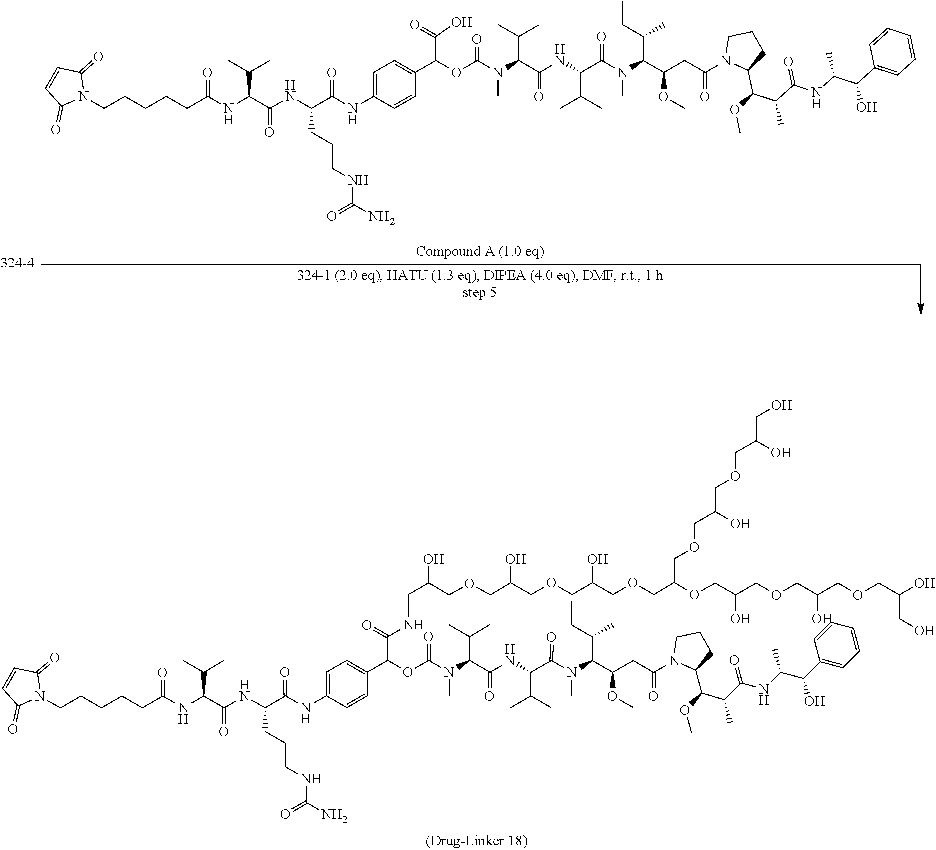

A solution of 323-4 (63.5 mg, 0.0883 mmol), Compound A (60 mg, 0.0441 mmol), HATU (21.8 mg, 0.0573 mmol) and DIPEA (22.8 mg, 0.176 mmol) in anhydrous DMF (4 mL) was stirred at room temperature for 1 h until Compound A was consumed. Then the reaction solution was purified by prep-HPLC to give Drug-Linker 18 (35.0 mg, 0.0173 mmol, 39.2%) as a white solid. LCMS, m/z=1013.85 (M/2+H)⁺.

### Example 19: Preparation of Drug-Linker 19

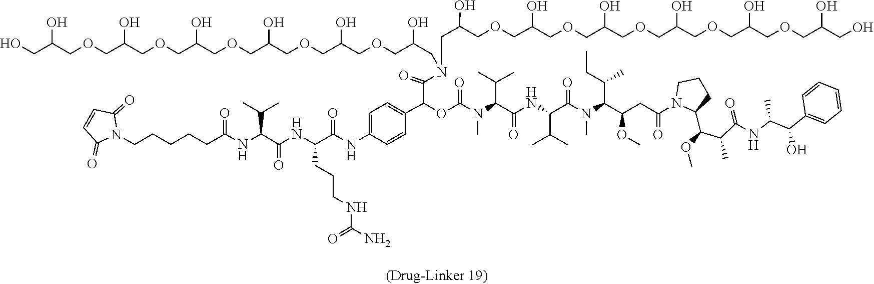 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(CC(O)COCC(O)COCC(O)COCC(O)COCC(O)COCC(O)CO)CC(O)COCC(O)COCC(O)COCC(O)COCC(O)COCC(O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

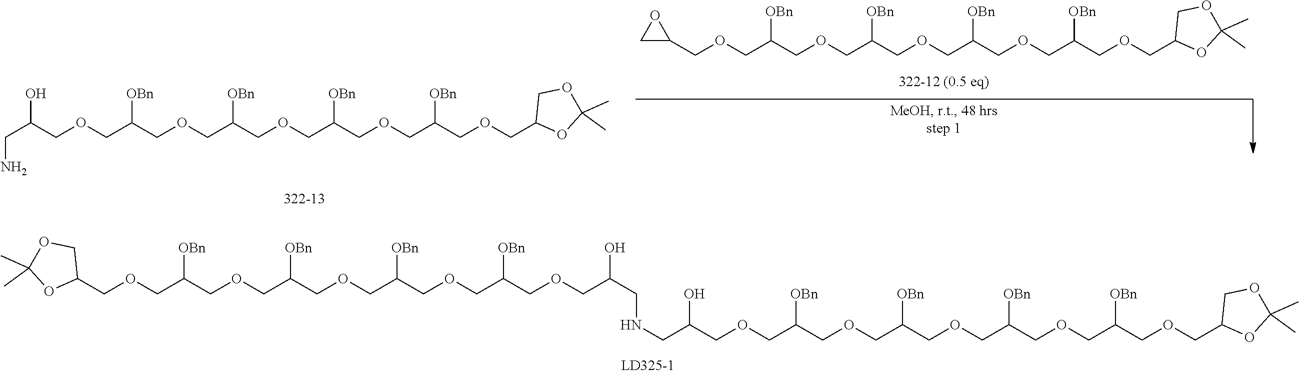

A solution of 322-13 (408 mg, 0.474 mmol), 322-12 (200 mg, 0.237 mmol) and MeOH (20 mL) was stirred at room temperature for 48 hrs. Then the reaction solution was purified by prep-HPLC to afford 325-1 (121.9 mg, 0.0715 mmol, 30.2%) as a colorless oil.

### Step 2

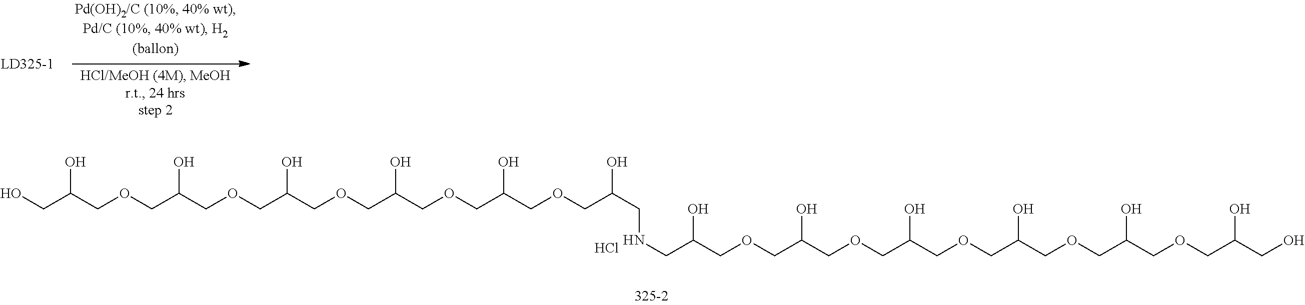 [SMILES: OCC(O)COCC(O)COCC(O)COCC(O)COCC(O)COCC(O)CNCC(O)COCC(O)COCC(O)COCC(O)COCC(O)COCC(O)CO ; Cl]

A mixture of 325-1 (121.9 mg, 0.0715 mmol), Pd(OH)₂/C (10%, 50 mg) and Pd/C (10%, 50 mg) in a mixed solvent of MeOH (10 mL) and HCl/MeOH (4M, 3 mL) was stirred under hydrogen atmosphere (ballon) for 24 h at room temperature until 325-1 was completely converted into 325-2. The reaction mixture was filtered through a celite pad, and the filtrate was concentrated to dryness under reduced pressure to afford 323-4 (67.2 mg, 0.0714 mmol, 100%) as an off-white solid, which was used in the next step without further purification.

### Step 3

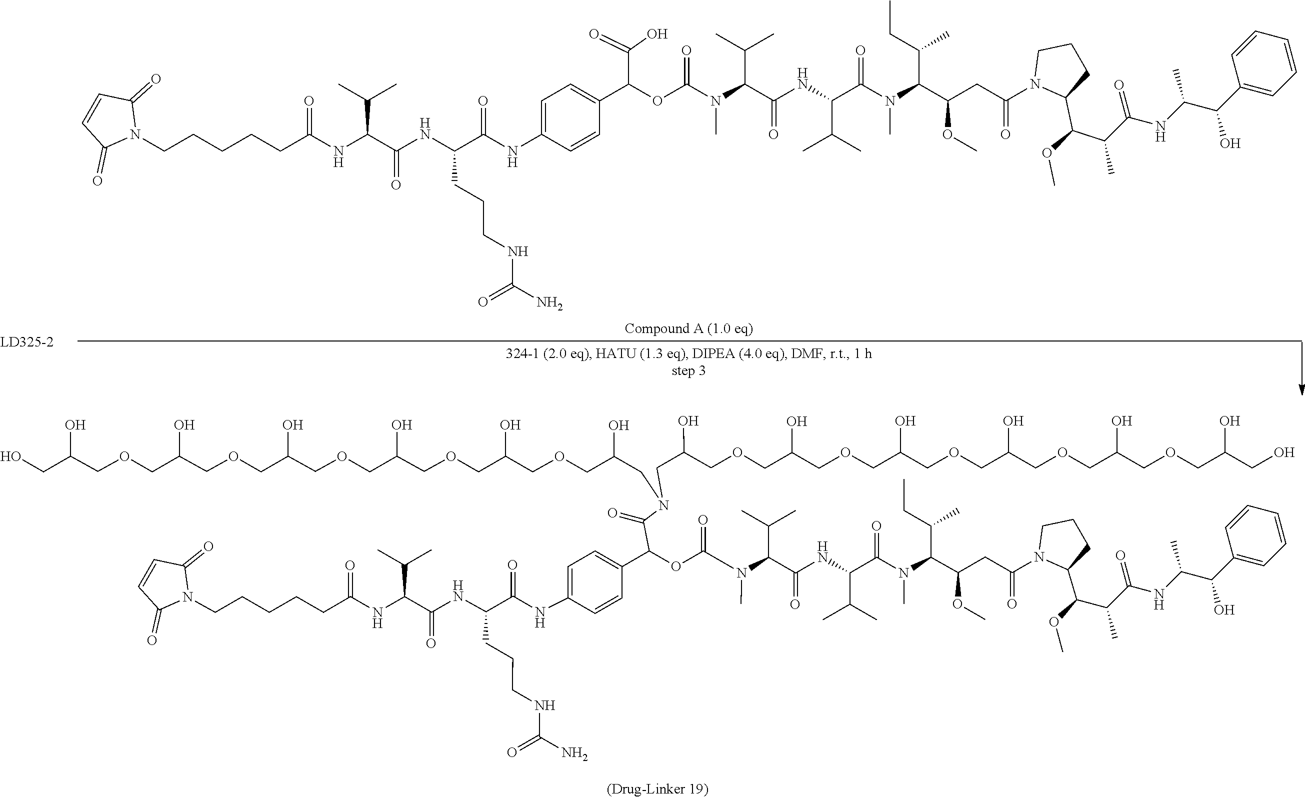

A solution of 325-2 (67.2 mg, 0.0714 mmol), Compound A (60 mg, 0.0441 mmol), HATU (21.8 mg, 0.0573 mmol) and DIPEA (22.8 mg, 0.176 mmol) in anhydrous DMF (4 mL) was stirred at room temperature for 1 h until Compound A was consumed. Then the reaction solution was purified by prep-HPLC to give Drug-Linker 19 (25.0 mg, 0.0111 mmol, 25.2%) as a white solid. LCMS, m/z=1124.79 (M/2+H)⁺.

### Example 20: Preparation of Drug-Linker 20

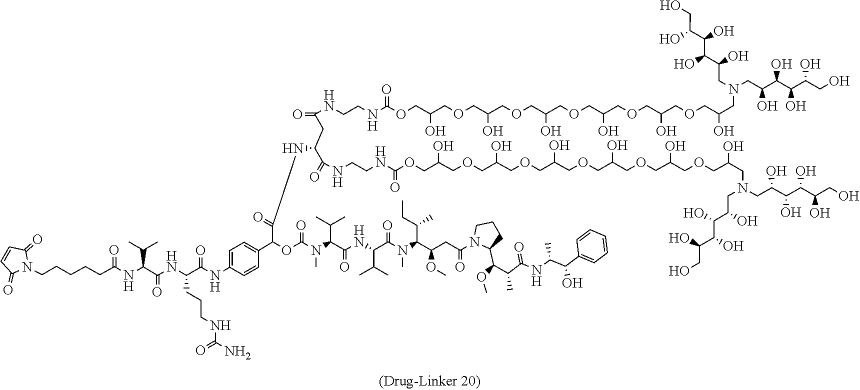 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@H](CC(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

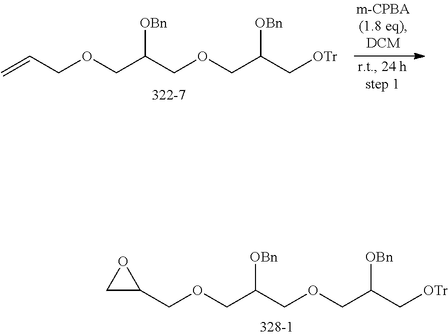

To a solution of 322-7 (10.0 g, 15.915 mmol) in DCM (20 mL) was added a solution of m-CPBA (4.94 g, 28.648 mmol) in DCM (40 mL) dropwise at room temperature. The resulting mixture was stirred at this temperature for 24 hours until 322-7 was consumed, and the reaction was quenched with saturated Na₂S₂O₃ (aq., 20 mL) and NaHCO₃ (aq, 20 mL). The reaction mixture was stirred for 30 mins, and then diluted with DCM (100 mL). The organic phase was washed with a mixture solution (20 mL×3) of saturated Na₂S₂O₃ and NaHCO₃ (aq, 1:1, V/V), dried over anhydrous Na₂SO₄, filtered and concentrated to dryness under reduced pressure to give the crude product, which was purified with column chromatography (silica, 0-90% ethyl acetate in petroleum ether) affording 328-1 (9.1 g, 14.12 mmol, 88.7%) as a pale yellow oil. Purity=90%-95%. ¹H NMR (400 MHz, CDCl₃) δ 7.47-7.38 (m, 5H), 7.36-7.03 (m, 20H), 4.70-4.39 (m, 4H), 3.79-3.41 (m, 9H), 3.40-3.32 (m, 1H), 3.28-3.14 (m, 2H), 3.12-3.01 (m, 1H), 2.79-2.66 (m, 1H), 2.60-2.47 (m, 1H).

### Step 2

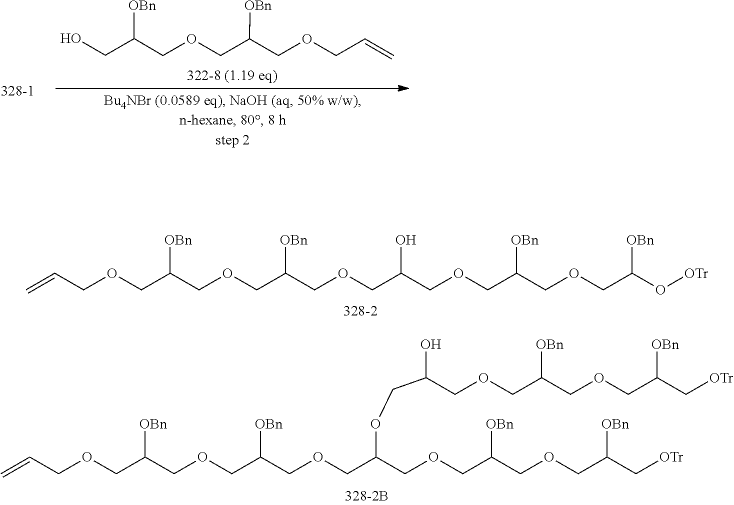

A solution of 322-8 (4.279 g, 11.08 mmol) and Bu₄NBr (177 mg, 0.548 mmol) in mixture solvent of n-hexane (20 mL) and NaOH (aq, 50% w/w, 20 mL) was stirred at 80° C. A mixture of 328-1 (6.0 g, 9.312 mmol) in n-hexane (8 mL) was dropped to the reaction mixture, and the resulting mixture was stirred at 80° C. for 8 hrs until most of 328-1 was consumed by TLC. The mixture was cooled down to r.t. and diluted with water (30 mL), then it was extracted with MTBE (70 mL×3). The organic parts were combined, dried over anhydrous Na₂SO₄, filtered and concentrated to dryness under reduced pressure. The crude product was purified with column chromatography (silica, 0-100% Ethyl acetate in petroleum ether) affording 328-2 (7.22 g, contains 328-2B) as a pale yellow oil. ¹H NMR (400 MHz, CDCl₃) δ 7.48-7.41 (m, 6H), 7.37-7.24 (m, 29H), 5.95-5.81 (m, 1H), 5.29-5.22 (m, 1H), 5.16 (dd, J=10.4, 1.7 Hz, 1H), 4.69-4.63 (m, 6H), 4.62-4.58 (m, 2H), 4.00-3.95 (m, 2H), 3.92-3.85 (m, 1H), 3.76-3.70 (m, 3H), 3.69-3.64 (m, 1H), 3.62-3.50 (m, 15H), 3.48-3.39 (m, 4H), 3.23 (d, J=5.2 Hz, 2H).

### Step 3

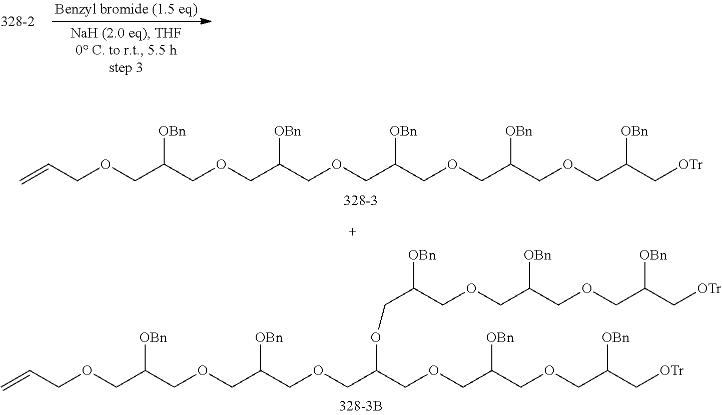

A mixture of NaH (560.5 mg, 14.012 mmol) in anhydrous THF (23.5 mL) was stirred in an ice bath, then a solution of 328-2 (7.22 g, 7.006 mmol, contains 328-2B) in anhydrous THF (13.2 mL) was added dropwise. The reaction mixture was stirred at this temperature for 20 mins, then benzyl bromide (1.786 g, 10.509 mmol) was added. The resulting mixture was allowed to warm to r.t. and stirred for 5 hrs until 328-2 was consumed by TLC. The reaction was quenched with saturated ammonium (20 mL) and extracted with ethyl acetate (40 mL×2). The organic phase was combined, dried over anhydrous Na₂SO₄, filtered and concentrated to dryness under reduced pressure to give the crude product, which was purified with column chromatography (silica, 0-80% Ethyl acetate in petroleum ether) affording 328-3 (5.13 g, 4.578 mmol, 65.3%) as a pale yellow oil and 328-3B (2.0 g, 1.133 mmol) a pale yellow oil. Purity=90%-95%. 1H NMR (400 MHz, CDCl₃) δ 7.46-7.42 (m, 6H), 7.33-7.20 (m, 34H), 5.94-5.81 (m, 1H), 5.27-5.22 (m, 1H), 5.17-5.12 (m, 1H), 4.67-4.63 (m, 8H), 4.61 (s, 2H), 3.99-3.95 (m, 2H), 3.75-3.66 (m, 5H), 3.60-3.50 (m, 18H), 3.23 (d, J=5.2 Hz, 2H). 1H NMR (400 MHz, CDCl₃) δ 7.46-7.40 (m, 13H), 7.34-7.25 (m, 27H), 7.24-7.15 (m, 25H), 5.93-5.81 (m, 1H), 5.26-5.21 (m, 1H), 5.14 (dd, J=10.4, 1.6 Hz, 1H), 4.67-4.65 (m, 2H), 4.64-4.61 (m, 8H), 4.60-4.57 (m, 4H), 3.99-3.93 (m, 2H), 3.75-3.63 (m, 10H), 3.59-3.55 (m, 6H), 3.54-3.46 (m, 20H), 3.21 (d, J=4.8 Hz, 4H).

### Step 4

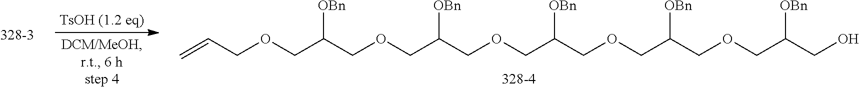 [SMILES: C=CCOCC(COCC(COCC(COCC(COCC(CO)OCc1ccccc1)OCc1ccccc1)OCc1ccccc1)OCc1ccccc1)OCc1ccccc1]

To a solution of 328-3 (3.6 g, 3.213 mmol) in a mixture solvent of DCM (22 mL) and MeOH (11 mL) was added TsOH·H₂O (733 mg, 3.855 mmol), the resulting mixture was stirred for 6 hrs at room temperature until 328-3 was consumed by TLC. The reaction was quenched with saturated NaHCO₃ (aq.), diluted with water (20 mL), and then extracted with DCM (40 mL×3). The organic phase was combined, dried over anhydrous Na₂SO₄, filtered and concentrated to dryness under reduced pressure to give the crude product, which was purified with column chromatography (silica, 0-60% ethyl acetate in petroleum ether) affording 328-4 (2.84 g, 23.233 mmol, 100%) as a colorless oil. Purity=90%-95%. ¹H NMR (400 MHz, CDCl₃) δ 7.38-7.26 (m, 25H), 5.96-5.84 (m, 1H), 5.27 (dq, J=17.2, 1.8 Hz, 1H), 5.17 (dq, J=10.4, 1.6 Hz, 1H), 4.73-4.63 (m, 10H), 4.63-4.58 (m, 1H), 3.99 (dt, J=5.6, 1.6 Hz, 2H), 3.78-3.71 (m, 5H), 3.67-3.63 (m, 2H), 3.62-3.54 (m, 18H).

### Step 5

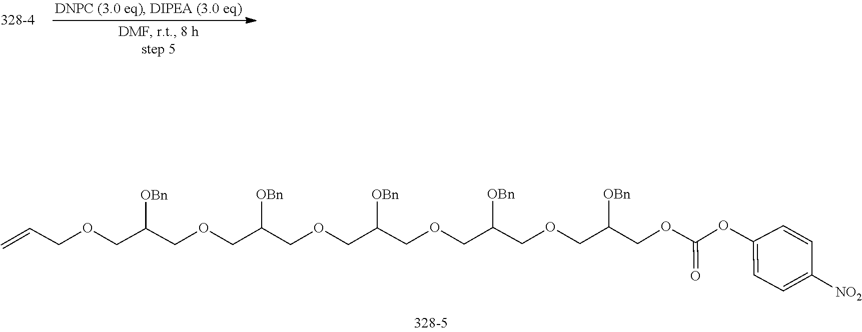 [SMILES: C=CCOCC(COCC(COCC(COCC(COCC(COC(=O)Oc1ccc([N+](=O)[O-])cc1)OCc1ccccc1)OCc1ccccc1)OCc1ccccc1)OCc1ccccc1)OCc1ccccc1]

To a solution of 328-4 (3.6 g, 3.213 mmol) in DMF (8 mL) was added DIPEA (353.2 mg, 2.733 mmol), followed by 4,4′-dinitrodiphenyl carbonate (831.1 mg, 2.732 mmol), then the resulting mixture was stirred at room temperature for 8 hrs until 328-4 was consumed detected by LCMS. The reaction solution was directly used in the next step without work-up procedure.

### Step 6

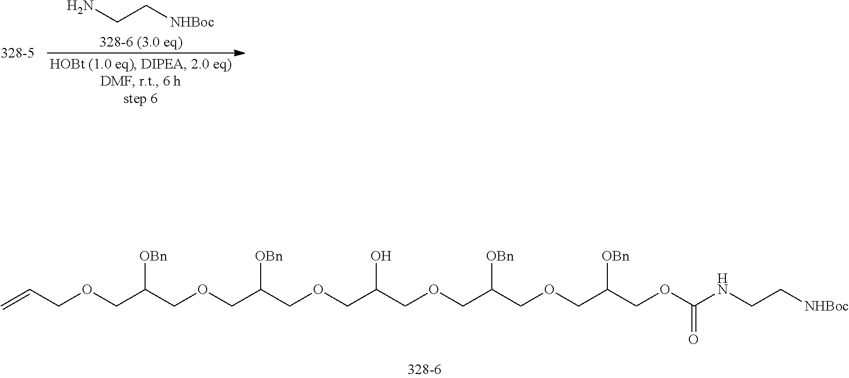 [SMILES: CC(C)(C)OC(=O)NCCN ; C=CCOCC(COCC(COCC(O)COCC(COCC(COC(=O)NCCNC(=O)OC(C)(C)C)OCc1ccccc1)OCc1ccccc1)OCc1ccccc1)OCc1ccccc1]

To the above reaction mixture was added HOBt (123 mg, 0.911 mmol), DIPEA (235.5 mg, 1.822 mmol) and 328-6 (437.6 mg, 2.733 mmol) successively, and the resulting mixture was stirred at room temperature for 6 hrs until 328-5 was consumed detected by LCMS. The reaction mixture was diluted with ethyl acetate (100 mL) and washed with saturated NaHCO₃ (aq, 30 mL×3), dried over anhydrous Na₂SO₄, filtered and concentrated to dryness under reduced pressure. The crude product was purified with column chromatography (silica, 0-90% ethyl acetate in petroleum ether) affording 328-6 (958 mg, 0.90 mmol, 98.7% over 2 steps) as a pale yellow oil. 1H NMR (400 MHz, CDCl₃) δ 7.36-7.22 (m, 25H), 5.95-5.80 (m, 1H), 5.28-5.21 (m, 1H), 5.20-5.13 (m, 1H), 5.07 (s, 1H), 4.83 (s, 1H), 4.69-4.62 (m, 10H), 4.30-4.21 (m, 1H), 4.16-4.10 (m, 1H), 3.97 (dt, J=5.6, 1.6 Hz, 2H), 3.77-3.69 (m, 5H), 3.62-3.50 (m, 19H), 3.27-3.10 (m, 4H), 1.43 (s, 9H),

### Step 7

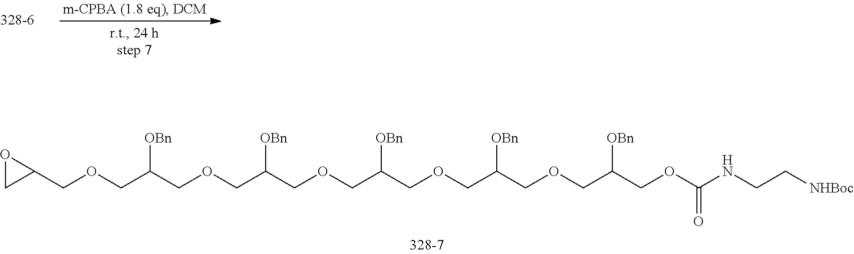 [SMILES: CC(C)(C)OC(=O)NCCNC(=O)OCC(COCC(COCC(COCC(COCC(COCC1CO1)OCc1ccccc1)OCc1ccccc1)OCc1ccccc1)OCc1ccccc1)OCc1ccccc1]

To a solution of 328-6 (958 mg, 0.90 mmol) in DCM (6 mL) was added a solution of m-CPBA (280 mg, 1.62 mmol) in DCM (6 mL) dropwise at room temperature. The resulting mixture was stirred at this temperature for 24 hours until 328-6 was consumed, and the reaction was quenched with saturated Na₂S₂O₃ (aq, 5 mL) and NaHCO₃ (aq., 5 mL). The reaction mixture was stirred for 30 mins, and then diluted with DCM (30 mL). The organic phase was washed with a mixture solution (10 mL×3) of saturated Na₂S₂O₃ and NaHCO₃ (aq, 1:1, V/V), dried over anhydrous Na₂SO₄, filtered and concentrated to dryness under reduced pressure to give the crude product, which was purified with column chromatography (silica, 0-100% ethyl acetate in petroleum ether) affording 328-7 (787 mg, 0.728 mmol, 80.9%) as a pale yellow oil. Purity=90%-95%.

### Step 8

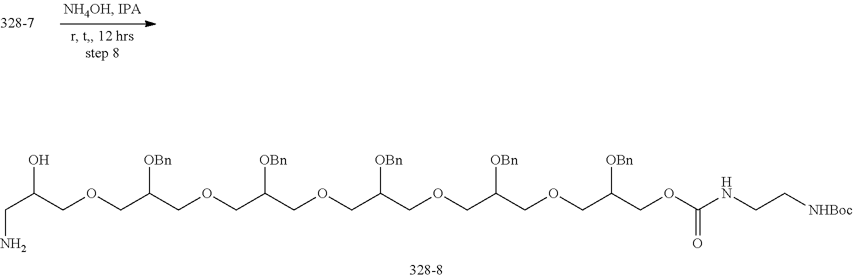 [SMILES: CC(C)(C)OC(=O)NCCNC(=O)OCC(COCC(COCC(COCC(COCC(COCC(O)CN)OCc1ccccc1)OCc1ccccc1)OCc1ccccc1)OCc1ccccc1)OCc1ccccc1]

To a solution of 328-7 (565 mg, 0.523 mmol) in isopropyl alcohol (43 mL) was added ammonia (43 mL) dropwise at room temperature, and the resulting mixture was stirred at this temperature for 12 hours until 328-7 was consumed. The reaction mixture was concentrated to dryness under reduced pressure to afford 328-8 (552.6 mg, 0.503 mmol, 96.3%) as a yellow oil, which was used in the next step without further purification. Purity=90%-95%.

### Step 9

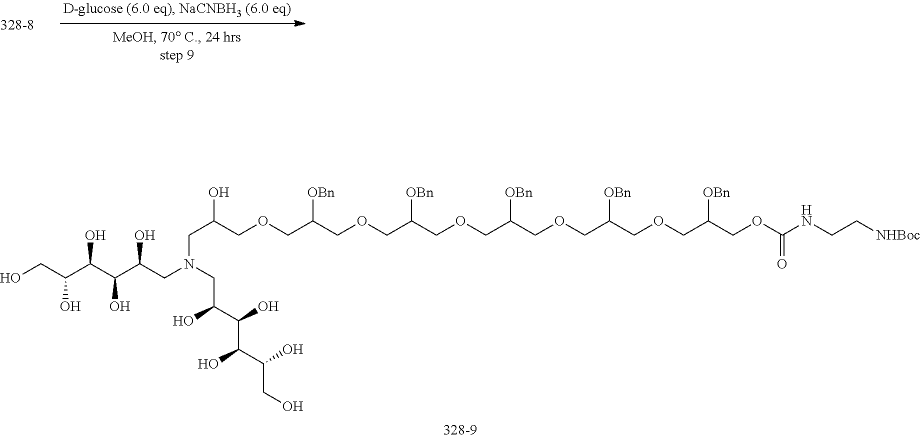 [SMILES: CC(C)(C)OC(=O)NCCNC(=O)OCC(COCC(COCC(COCC(COCC(COCC(O)CN(C[C@H](O)[C@@H](O)[C@H](O)[C@H](O)CO)C[C@H](O)[C@@H](O)[C@H](O)[C@H](O)CO)OCc1ccccc1)OCc1ccccc1)OCc1ccccc1)OCc1ccccc1)OCc1ccccc1]

A mixture of 328-8 (552.6 mg, 0.503 mmol), D-glucose (544.2 mg, 3.021 mmol) and NaCNBH₃ (189.8 mg, 3.021 mmol) in anhydrous MeOH (12 mL) was stirred at 70° C. for 24 hrs until most of 328-8 was consumed and 328-9 was detected by LCMS. The reaction mixture was cooled down to room temperature, filtered and concentrated under reduced pressure to give the crude product, which was purified by reverse phase liquid chromatography to give 328-9 (597 mg, 0.419 mmol, 83.2%) as a colorless oil. Purity=90%-95%. ¹H NMR (400 MHz, MeOH-d₄) δ 7.41-7.20 (m, 25H), 4.69-4.57 (m, 10H), 4.26-4.04 (m, 5H), 3.84-3.78 (m, 3H), 3.77-3.68 (m, 8H), 3.67-3.42 (m, 28H), 3.38-3.34 (m, 1H), 3.33-3.31 (m, 1H), 3.17-3.06 (m, 4H), 1.40 (s, 9H).

### Step 10

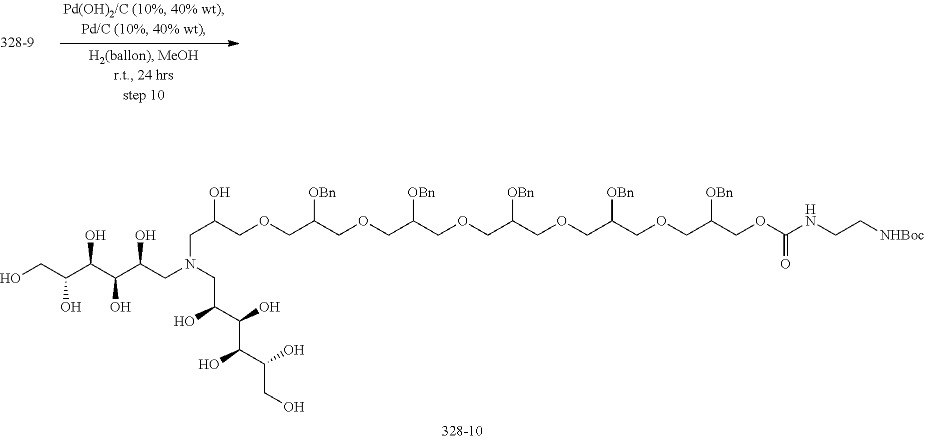 [SMILES: CC(C)(C)OC(=O)NCCNC(=O)OCC(COCC(COCC(COCC(COCC(COCC(O)CN(C[C@H](O)[C@@H](O)[C@H](O)[C@H](O)CO)C[C@H](O)[C@@H](O)[C@H](O)[C@H](O)CO)OCc1ccccc1)OCc1ccccc1)OCc1ccccc1)OCc1ccccc1)OCc1ccccc1]

A mixture of 328-9 (376 mg, 0.264 mmol), Pd(OH)₂/C (10%, 150 mg) and Pd/C (10%, 150 mg) in MeOH (30 mL) was stirred under hydrogen atmosphere (ballon) for 24 h at room temperature until 328-9 was completely converted into 328-10. The reaction mixture was filtered through a celite pad, and the filtrate was concentrated to dryness under reduced pressure to afford 328-10 (211.4 mg, 0.217 mmol, 82.1%) as an off-white solid, which was used in the next step without further purification.

### Step 11

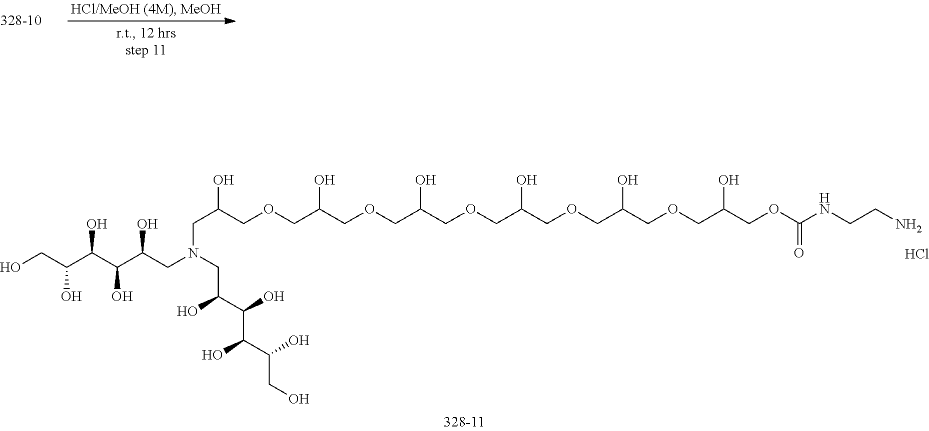 [SMILES: NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO ; Cl]

A solution of 328-10 (211.4 mg, 0.216 mmol) in HCl/MeOH (4M, 3 mL) and MeOH (3 mL) was stirred at room temperature for 12 hours until 328-10 was completely converted into 328-11. The reaction mixture was concentrated to dryness under reduced pressure to afford 328-11 (198.0 mg, 0.217 mmol, 100%) as an off-white solid, which was used in the next step without further purification.

### Step 12

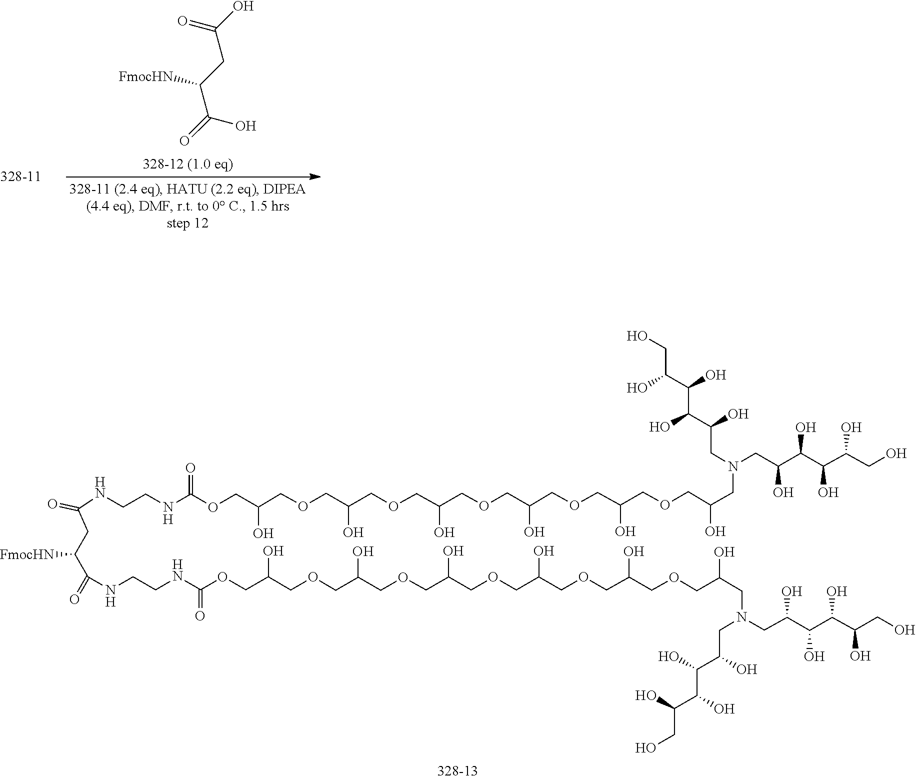

A solution of 328-12 (24.8 mg, 0.0698 mmol) and HATU (58.4 mg, 0.153 mmol) in anhydrous DMF (2 mL) was stirred at room temperature for 15 mins, then it was stirred in an ice bath. A solution of 328-11 (150 mg, 0.168 mmol) in anhydrous DMF (2 mL) was added dropwise, followed by DIPEA (39.7 mg, 0.307 mmol). The resulting mixture was stirred in the ice bath for 1 h until most of 328-12 was consumed. The reaction mixture was purified by reverse phase liquid chromatography to give 328-13 (97.5 mg, 0.0471 mmol, 67.4%) as a colorless oil. Purity=90%-95%.

### Step 13

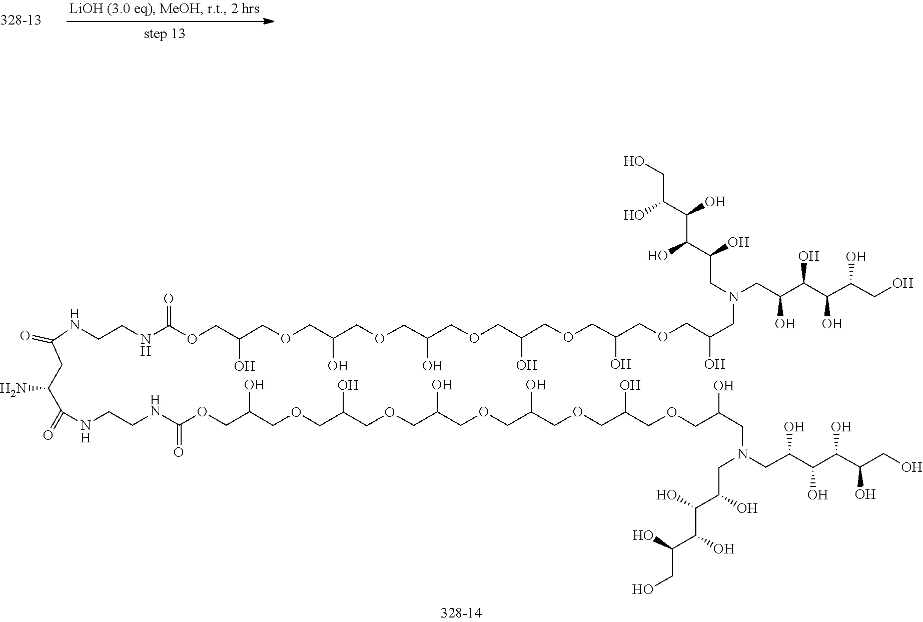 [SMILES: N[C@H](CC(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO]

To a solution of 328-13 (97.5 mg, 0.0471 mmol) in MeOH (3 mL) was added LiOH·H₂O (5.9 mg, 0.141 mmol), and the mixture was stirred at room temperature for 2 hrs until 328-13 was consumed. The reaction solution was neutralized with 1N HCl to pH=7, and concentrated under reduced pressure to give a crude product, which was dissolved in H₂O (10 mL) and washed with hexane (5 mL×3). The aqueous phase was concentrated to dryness under reduced pressure to afford 328-14 (87.0 mg, 0.047 mmol, 100%) as a colorless oil, which was used in the next step without further purification.

### Step 14

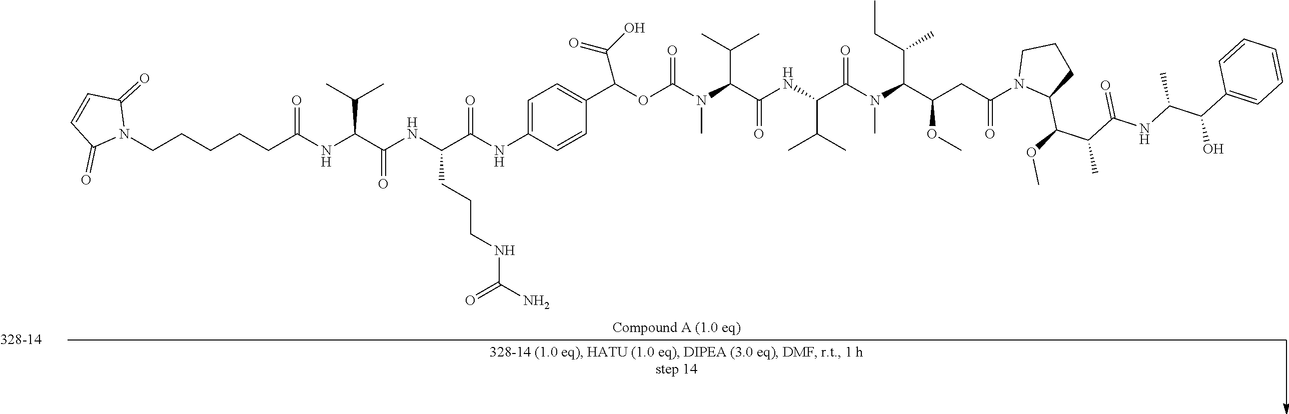 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)O)c1ccc(NC(=O)[C@H](CCCNC(N)=O)NC(=O)[C@@H](NC(=O)CCCCCN2C(=O)C=CC2=O)C(C)C)cc1)C(C)C]

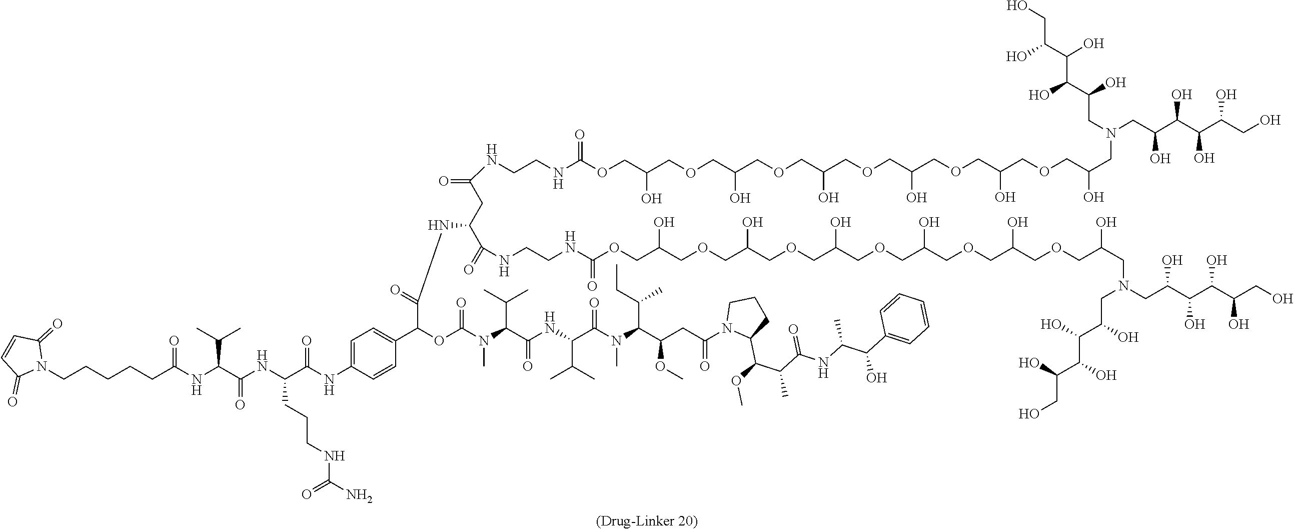 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@H](CC(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)C(O)[C@H](O)CO)C(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

A solution of 328-14 (85 mg, 0.0460 mmol), Compound A (62 mg, 0.0460 mmol), HATU (17.2 mg, 0.0452 mmol) and DIPEA (17.9 mg, 0.139 mmol) in anhydrous DMF (4 mL) was stirred at room temperature for 1 h until Compound A was consumed. Then the reaction solution was purified by prep-HPLC to give Drug-Linker 20 (73.0 mg, 0.0229 mmol, 49.8%) as a white solid. LCMS, m/z=1595.97 (M/2+H)⁺, m/z=1064.29 (M/2+H)⁺. ¹H NMR (400 MHz, DMSO-d₆) δ 10.04 (s, 1H), 8.59-8.02 (m, 4H), 8.00-7.72 (m, 2H), 7.70-7.46 (m, 3H), 7.45-7.09 (m, 8H), 7.00 (s, 2H), 5.99 (s, 1H), 5.91-5.66 (m, 2H), 5.44 (s, 5H), 5.07-4.29 (m, 16H), 4.25-3.87 (m, 12H), 3.86-3.47 (m, 31H), 3.30-2.74 (m, 27H), 2.44-1.84 (m, 10H), 1.83-1.63 (m, 4H), 1.59-1.27 (m, 10H), 1.24-1.10 (m, 3H), 1.08-0.64 (m, 30H).

### Example 21: Preparation of Drug-Linker 21

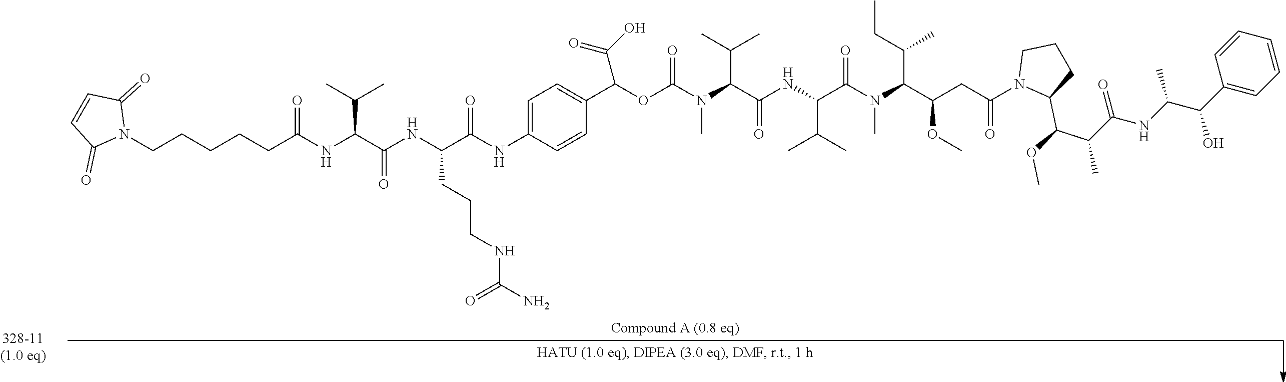 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)O)c1ccc(NC(=O)[C@H](CCCNC(N)=O)NC(=O)[C@@H](NC(=O)CCCCCN2C(=O)C=CC2=O)C(C)C)cc1)C(C)C]

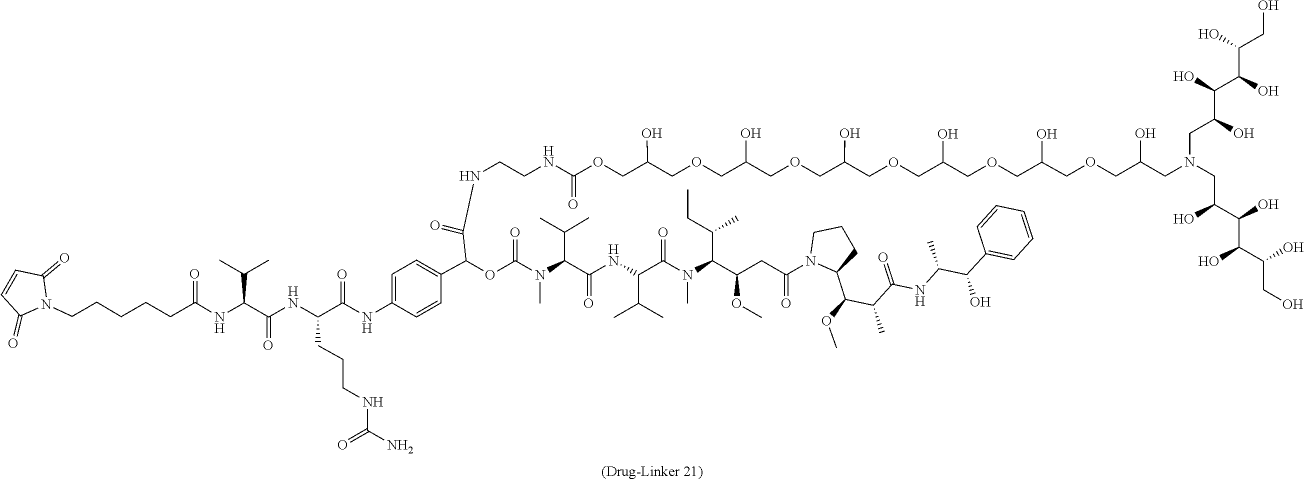

A solution of 328-11 (42 mg, 0.0461 mmol), Compound A (50 mg, 0.0368 mmol), HATU (17.4 mg, 0.0458 mmol) and DIPEA (17.8 mg, 0.138 mmol) in anhydrous DMF (3 mL) was stirred at room temperature for 1 h until Compound A was consumed. Then the reaction solution was purified by prep-HPLC to give Drug-Linker 21 (37.8 mg, 0.0170 mmol, 46.3%) as a white solid. LCMS, m/z=1109.75 (M/2+H)⁺. ¹H NMR (400 MHz, D₂O) δ 7.60-7.43 (m, 4H), 7.39-7.27 (m, 4H), 7.25-7.14 (m, 1H), 6.78 (s, 2H), 6.05-5.89 (m, 1H), 4.78-4.54 (m, 6H), 4.52-3.90 (m, 28H), 3.89-3.71 (m, 13H), 3.69-3.38 (m, 64H), 3.37-2.85 (m, 25H), 2.81-2.60 (m, 3H), 2.58-2.40 (m, 2H), 2.37-2.11 (m, 4H), 2.08-1.96 (m, 2H), 1.94-1.70 (m, 5H), 1.66-1.45 (m, 8H), 1.35-1.12 (m, 8H), 1.10-0.73 (m, 25H), 0.67-0.50 (m, 1H), 0.47-0.35 (m, 1H).

### Example 22: Preparation of Drug-Linker 22

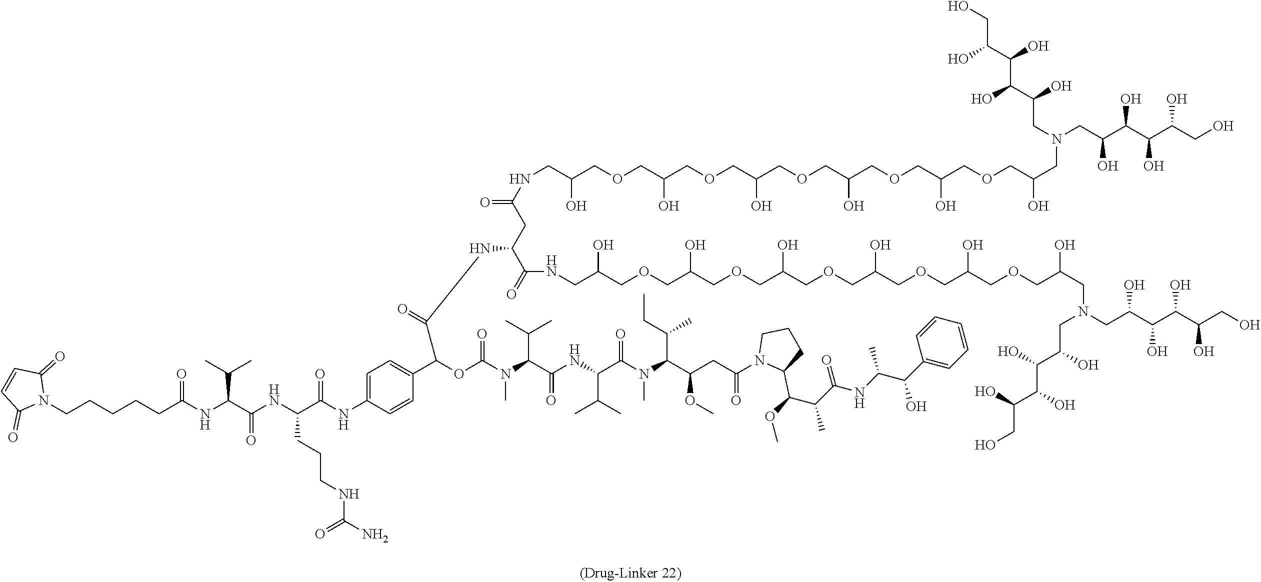 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@H](CC(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

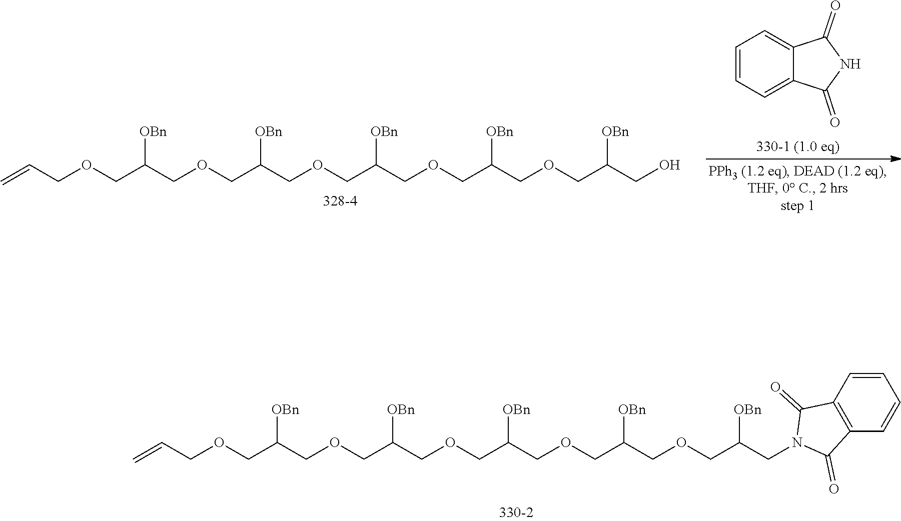 [SMILES: O=C1NC(=O)c2ccccc21 ; C=CCOCC(COCC(COCC(COCC(COCC(CO)OCc1ccccc1)OCc1ccccc1)OCc1ccccc1)OCc1ccccc1)OCc1ccccc1 ; C=CCOCC(COCC(COCC(COCC(COCC(CN1C(=O)c2ccccc2C1=O)OCc1ccccc1)OCc1ccccc1)OCc1ccccc1)OCc1ccccc1)OCc1ccccc1]

To a solution of 330-1 (217.6 mg, 1.48 mmol) and PPh₃ (465.5 mg, 1.776 mmol) in THF (8 mL) was added a solution of 328-4 (1.3 g, 1.48 mmol) in THF (4 mL) and the mixture was stirred in an ice bath. A solution of DEAD (309.3 mg, 1.776 mmol) in THF (1 mL) was added to the above solution and the resulting mixture was allowed to warm to r.t. and stirred for 2 hrs until 328-4 was consumed by TLC. The reaction was quenched with water (1 mL), and the reaction was concentrated under reduced pressure to give the crude product, which was purified with column chromatography (silica, 0-60% ethyl acetate in petroleum ether) to afford 330-2 (1.307 g, 1.297 mmol, 87.7%) as a white solid. Purity=90%-95%. 1H NMR (400 MHz, CDCl₃) δ 7.77 (dd, J=5.6, 3.2 Hz, 2H), 7.66 (dd, J=5.6, 3.2 Hz, 2H), 7.34-7.20 (m, 20H), 7.19-7.15 (m, 2H), 7.12-7.04 (m, 3H), 5.95-5.81 (m, 1H), 5.29-5.22 (m, 1H), 5.15 (d, J=10.4 Hz, 1H), 4.69-4.63 (m, 9H), 4.51 (d, J=12.0 Hz, 1H), 3.99-3.95 (m, 2H), 3.93-3.83 (m, 2H), 3.76-3.69 (m, 5H), 3.60-3.52 (m, 18H).

### Step 2

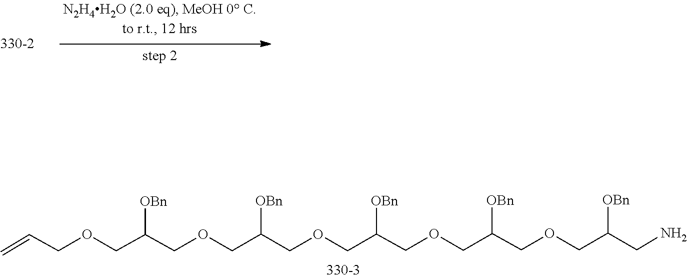 [SMILES: C=CCOCC(COCC(COCC(COCC(COCC(CN)OCc1ccccc1)OCc1ccccc1)OCc1ccccc1)OCc1ccccc1)OCc1ccccc1]

To a solution of 330-2 (1.472 g, 1.46 mmol) in MeOH (10 mL) was added N₂H₄-H₂O (146.3 mg, 2.92 mmol) in an ice bath, the resulting mixture was allowed to warm to r.t. and stirred for 10 hrs until 330-2 was consumed by TLC. The reaction mixture was concentrated to dryness under reduced pressure to give the crude product, which was dissolved in ethyl acetate (20 mL) and filtered. The filtrate was concentrated under reduced pressure to afford 330-3 (1.23 g, 1.402 mmol, 95.9%) as a colorless oil, which was used in the next step without further purification.

### Step 3

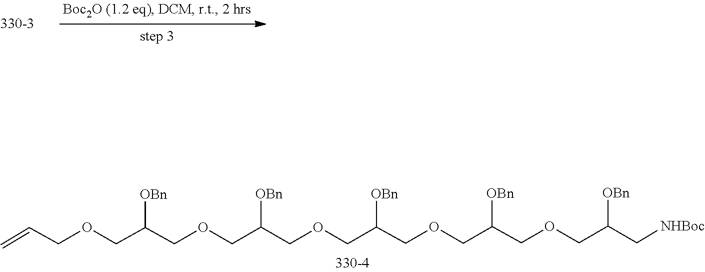 [SMILES: C=CCOCC(COCC(COCC(COCC(COCC(CNC(=O)OC(C)(C)C)OCc1ccccc1)OCc1ccccc1)OCc1ccccc1)OCc1ccccc1)OCc1ccccc1]

To a solution of 330-3 (1.23 g, 1.40 mmol) in DCM (8 mL) was added Boc₂O (367 mg. 1.68 mmol) at room temperature, and the reaction mixture was stirred at this temperature for 2 hrs until 330-3 was consumed and 330-4 was detected by LCMS. The reaction was concentrated under reduced pressure to give the crude product, which was purified with column chromatography (silica, 0-40% ethyl acetate in petroleum ether) affording 330-4 (1.23 g, 1.26 mmol, 89.8%) as a colorless oil. Purity=90%-95%.

### Step 4

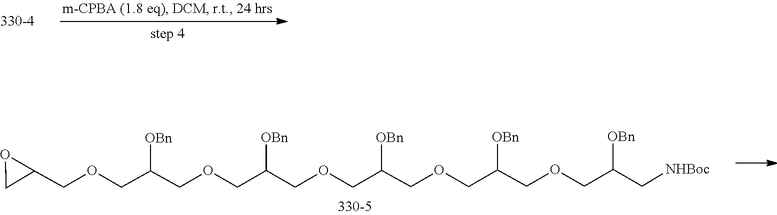 [SMILES: CC(C)(C)OC(=O)NCC(COCC(COCC(COCC(COCC(COCC1CO1)OCc1ccccc1)OCc1ccccc1)OCc1ccccc1)OCc1ccccc1)OCc1ccccc1]

To a solution of 330-4 (800 mg, 0.818 mmol) in DCM (5 mL) was added a solution of m-CPBA (254 mg, 1.473 mmol) in DCM (5 mL) at room-temperature, and the mixture was stirred for 24 hrs until 330-4 was consumed by TLC. The reaction was quenched by adding sat. Na₂S₂O₃ (5 mL) and sat. NaHCO₃ (5 mL), and the resulting mixture was stirred for 30 mins before diluting with DCM (60 mL). The organic phase was sequentially washed with sat. Na₂S₂O₃ (20 mL) and sat. NaHCO₃ (20 mL), dried over anhydrous Na₂SO₄, filtered and concentrated to dryness under reduced pressure. The crude product was purified with column chromatography (silica, 0-60% Ethyl acetate in petroleum ether) to afford 330-5 (689 mg, 0.693 mmol, 84.8%) as a colorless oil. Purity=90%-95%.

### Step 5

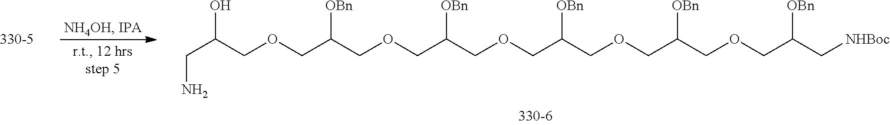 [SMILES: CC(C)(C)OC(=O)NCC(COCC(COCC(COCC(COCC(COCC(O)CN)OCc1ccccc1)OCc1ccccc1)OCc1ccccc1)OCc1ccccc1)OCc1ccccc1]

To a solution of 330-5 (689 mg, 0.693 mmol) in isopropyl alcohol (56 mL) was added ammonia (43 mL) dropwise at room temperature, and the resulting mixture was stirred at this temperature for 12 hours until 330-5 was consumed. The reaction mixture was concentrated to dryness under reduced pressure to afford 330-6 (698.7 mg, 0.691 mmol, 99.7%) as a yellow oil, which was used in the next step without further purification. Purity=90%-95%.

### Step 6

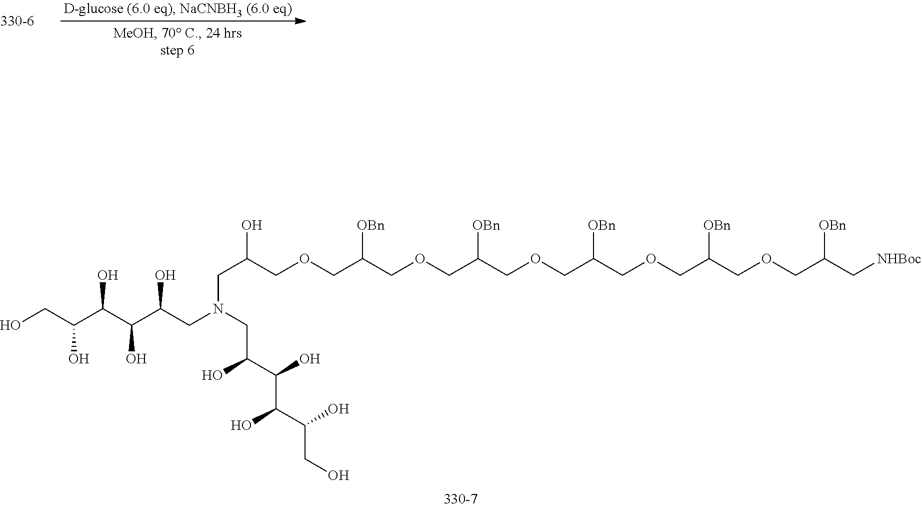 [SMILES: CC(C)(C)OC(=O)NCC(COCC(COCC(COCC(COCC(COCC(O)CN(C[C@H](O)[C@@H](O)[C@H](O)[C@H](O)CO)C[C@H](O)[C@@H](O)[C@H](O)[C@H](O)CO)OCc1ccccc1)OCc1ccccc1)OCc1ccccc1)OCc1ccccc1)OCc1ccccc1]

A mixture of 330-6 (983 mg, 0.973 mmol), D-glucose (1.05 g, 5.836 mmol) and NaCNBH₃ (366.7 mg, 5.836 mmol) in anhydrous MeOH (15 mL) was stirred at 70° C. for 24 hrs until most of 330-6 was consumed and 330-7 was detected by LCMS. The reaction mixture was cooled down to room temperature, filtered and concentrated under reduced pressure to give the crude product, which was purified by reverse phase liquid chromatography to give 330-7 (889 mg, 0.664 mmol, 68.2%) as a colorless oil. Purity=90%-95%.

### Step 7

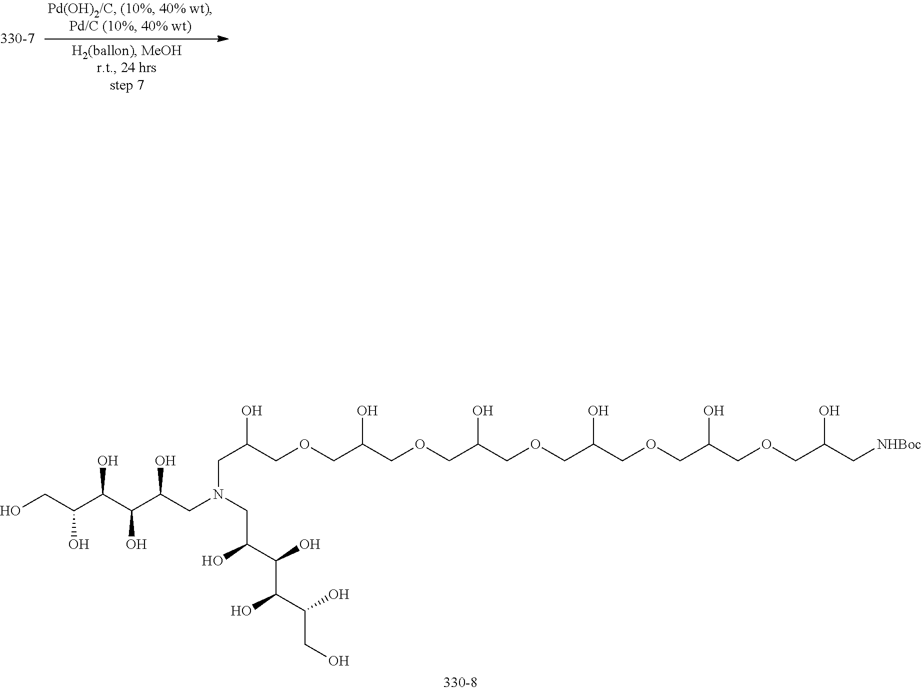 [SMILES: CC(C)(C)OC(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO]

A mixture of 330-7 (270 mg, 0.202 mmol), Pd(OH)₂/C (10%, 120 mg) and Pd/C (10%, 120 mg) in MeOH (25 mL) was stirred under hydrogen atmosphere (balloon) for 24 h at room temperature until 330-7 was completely converted into 330-8. The reaction mixture was filtered through a celite pad, and the filtrate was concentrated to dryness under reduced pressure to afford 330-8 (138.2 mg, 0.156 mmol, 77.0%) as an off-white solid, which was used in the next step without further purification.

### Step 8

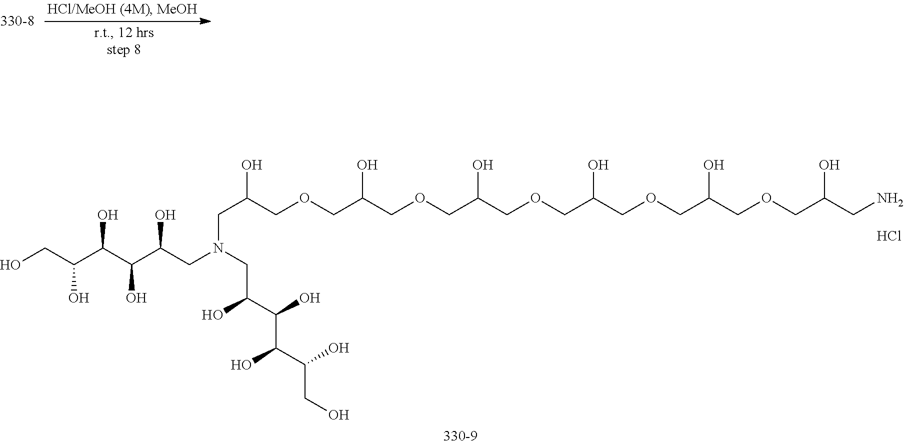 [SMILES: NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO ; Cl]

A solution of 330-8 (138 mg, 0.155 mmol) in HCl/MeOH (4M, 3 mL) and MeOH (3 mL) was stirred at room temperature for 12 hours until 330-8 was completely converted into 330-9. The reaction mixture was concentrated to dryness under reduced pressure to afford 330-9 (128 mg, 0.155 mmol, 100%) as an off-white solid, which was used in the next step without further purification.

### Step 9

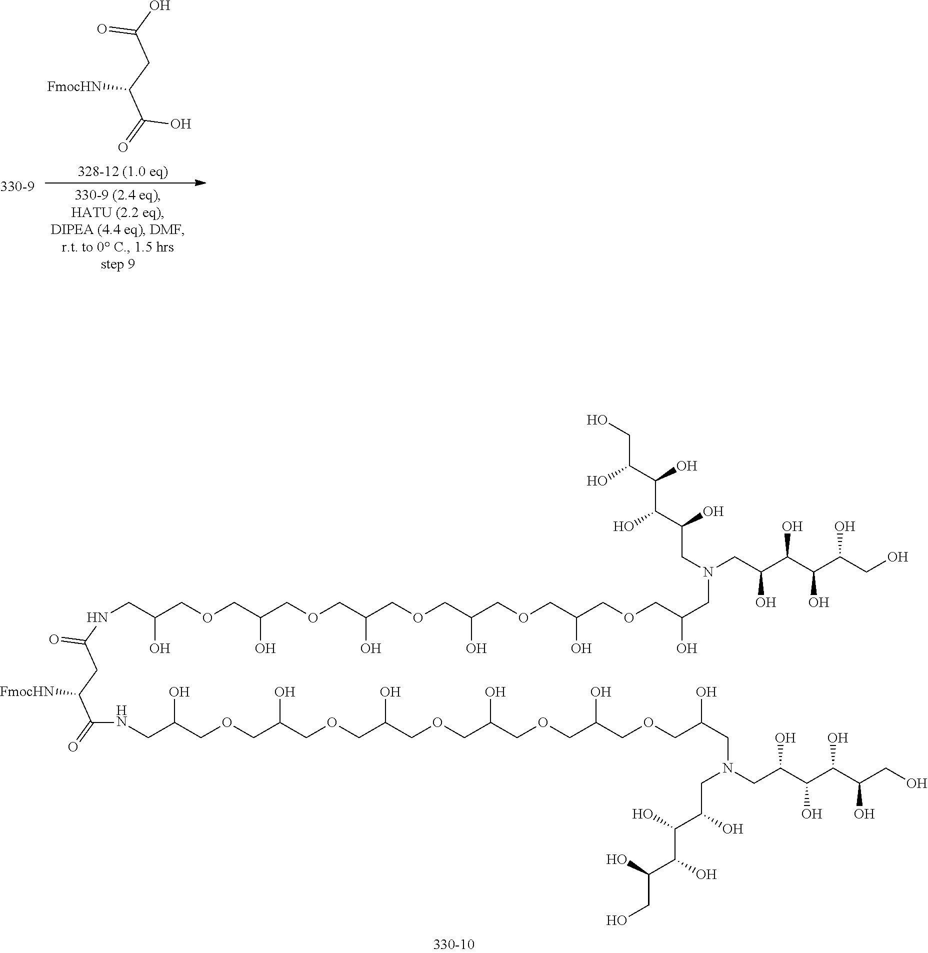

A solution of 328-12 (16.5 mg, 0.0465 mmol) and HATU (38.9 mg, 0.102 mmol) in anhydrous DMF (2 mL) was stirred at room temperature for 15 mins, then it was stirred in an ice bath. A solution of 330-9 (92 mg, 0.112 mmol) in anhydrous DMF (2 mL) was added dropwise, followed by DIPEA (26.5 mg, 0.205 mmol). The resulting mixture was stirred in the ice bath for 1 h until most of 328-12 was consumed. The reaction mixture was purified by reverse phase liquid chromatography to give 330-10 (14 mg, 0.00738 mmol, 15.9%) as a colorless oil.

### Step 10

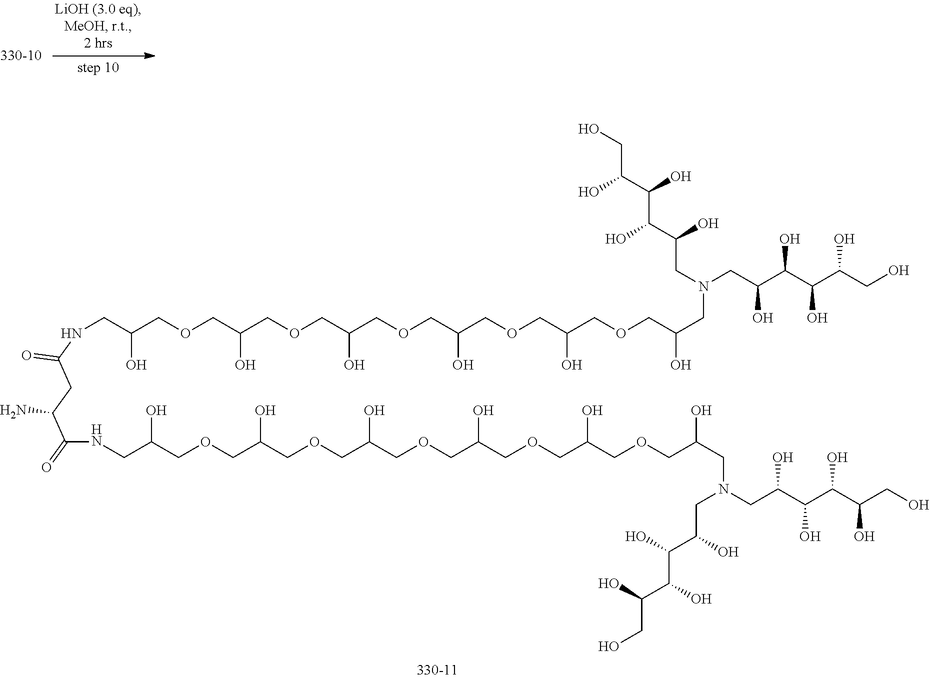 [SMILES: N[C@H](CC(=O)NCC(O)COCC(O)COCC(O)COCC(O)COCC(O)COCC(O)CN(C[C@H](O)[C@H](O)[C@H](O)[C@H](O)CO)C[C@H](O)[C@@H](O)[C@H](O)[C@H](O)CO)C(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO]

To a solution of 330-10 (14 mg, 0.00738 mmol) in MeOH (2 mL) was added LiOH·H₂O (2 mg, 0.0442 mmol), and the mixture was stirred at room temperature for 2 hrs until 330-10 was consumed. The reaction solution was neutralized with 1N HCl to pH=7, and concentrated under reduced pressure to give a crude product, which was dissolved in H₂O (5 mL) and washed with hexane (2 mL×3). The aqueous phase was concentrated to dryness under reduced pressure to afford 330-11 (12.4 mg, 0.0074 mmol, 100%) as a white solid, which was used in the next step without further purification.

### Step 11

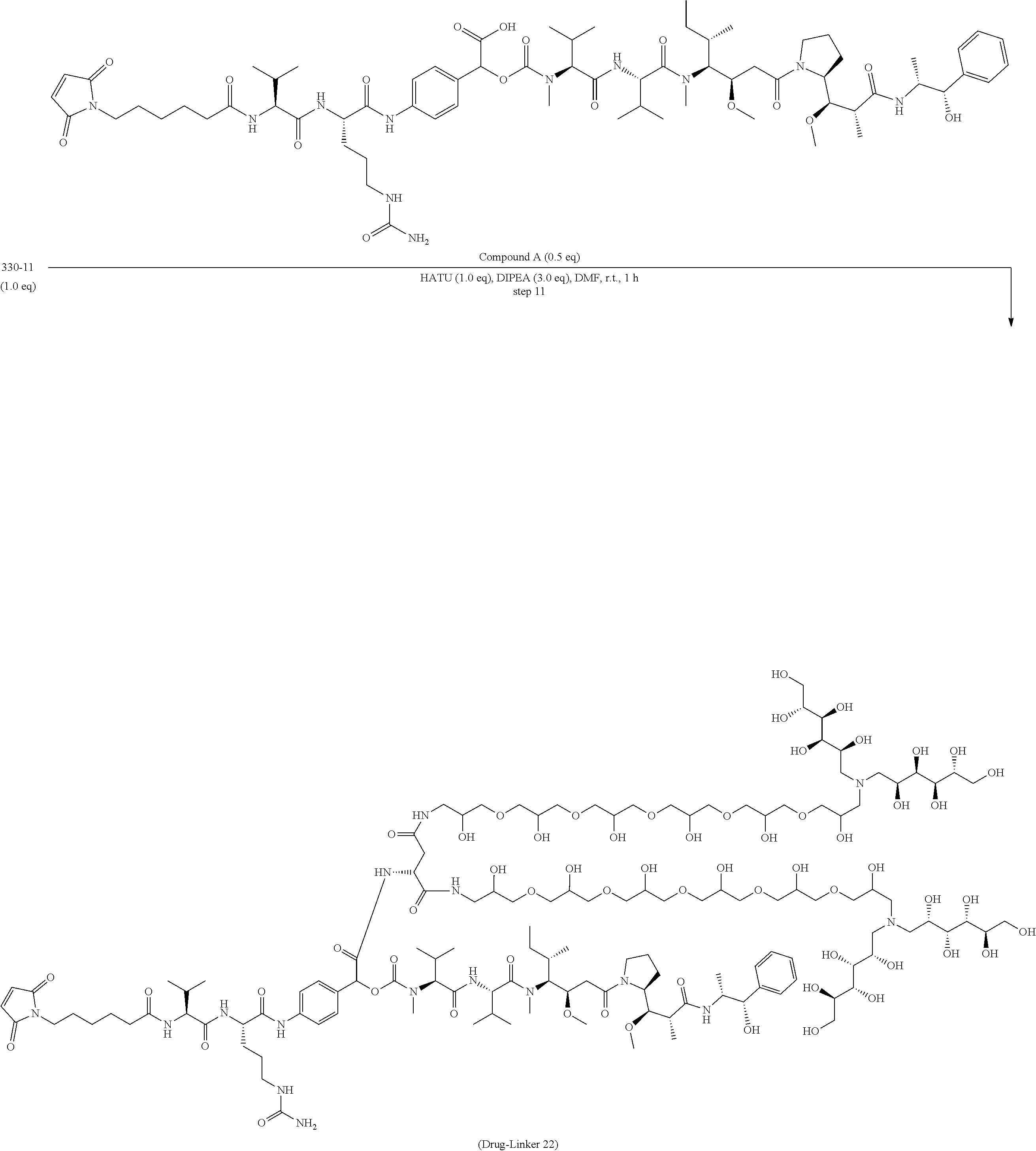

A solution of 330-11 (12 mg, 0.00716 mmol), Compound A (5.0 mg, 0.00368 mmol), HATU (2.7 mg, 0.0071 mmol) and DIPEA (2.8 mg, 0.0217 mmol) in anhydrous DMF (2 mL) was stirred at room temperature for 1 h until Compound A was consumed. Then the reaction solution was purified by prep-HPLC to give Drug-Linker 22 (5.0 mg, 0.00166 mmol, 45.1%) as a white solid. LCMS, m/z=1509.00 (M/2+H)⁺, m/z=1006.6 (M/3+H)⁺.

### Example 23: Preparation of Drug-Linker 23

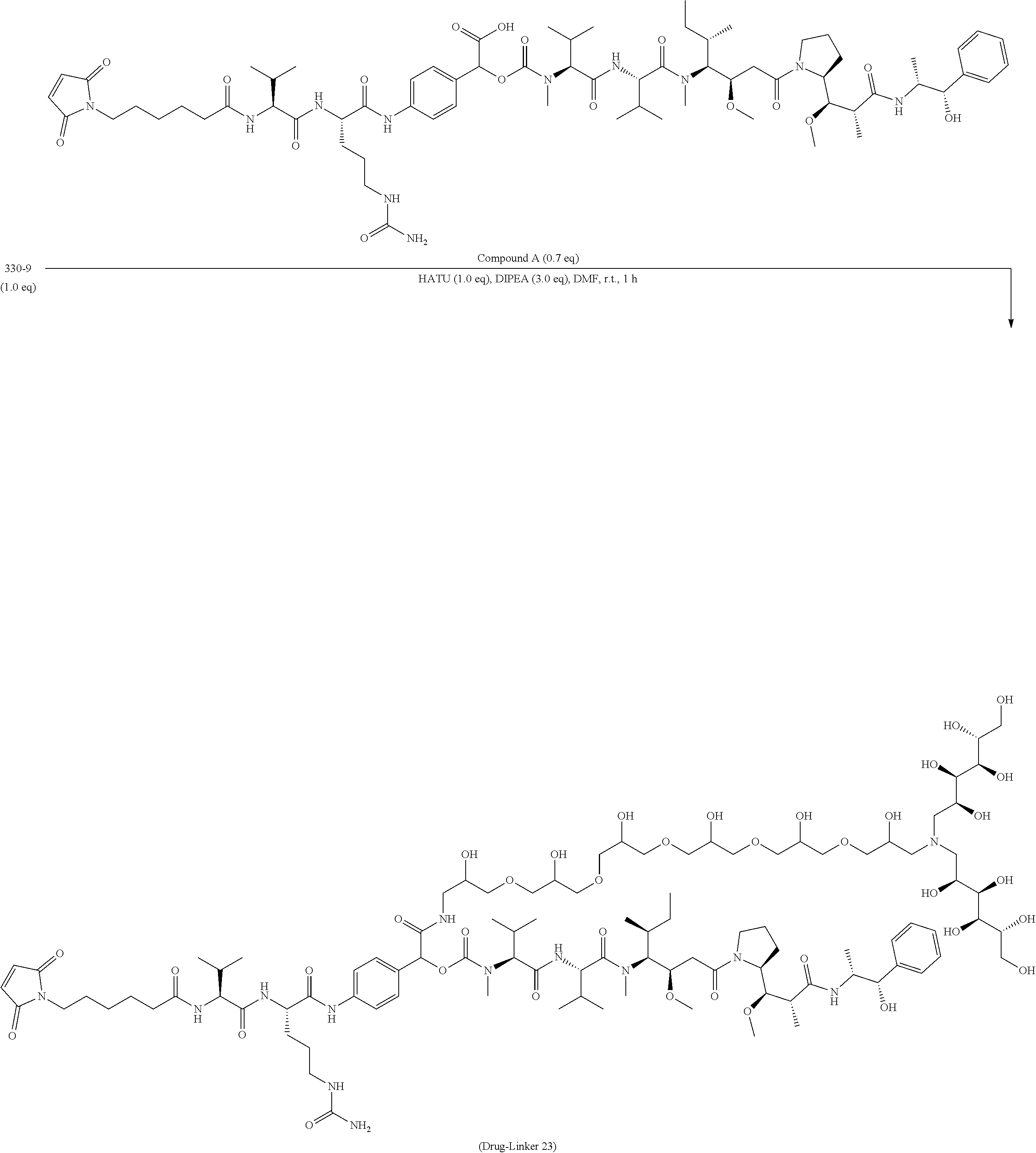

A solution of 330-9 (46 mg, 0.0558 mmol), Compound A (53 mg, 0.0390 mmol), HATU (21.2 mg, 0.0558 mmol) and DIPEA (21.6 mg, 0.167 mmol) in anhydrous DMF (4 mL) was stirred at room temperature for 1 h until Compound A was consumed. Then the reaction solution was purified by prep-HPLC to give Drug-Linker 23 (28 mg, 0.0131 mmol. 33.7%) as a white solid. LCMS, m/z=1066.11 (M/2+H)⁺. ¹H NMR (400 MHz, D₂O) δ 7.50-7.42 (m, 4H), 7.40-7.26 (m, 4H), 7.25-7.13 (m, 1H), 6.77 (s, 2H), 6.04-5.87 (m, 1H), 4.72-4.55 (m, 2H), 4.52-4.40 (m, 2H), 4.37-4.17 (m, 5H), 4.13-3.96 (m, 5H), 3.94-3.85 (m, 2H), 3.84-3.72 (m, 6H), 3.69-3.21 (m, 43H), 3.18-2.90 (m, 8H), 2.71 (s, 1H), 2.61-2.39 (m, 2H), 2.35-2.11 (m, 4H), 2.04 (d, J=9.8 Hz, 2H), 1.93-1.71 (m, 5H), 1.67-1.44 (m, 8H), 1.34-1.26 (m, 3H), 1.25-1.11 (m, 5H), 1.06 (d, J=6.4 Hz, 2H), 0.99-0.72 (m, 22H), 0.62-0.52 (m, 1H), 0.47-0.33 (m, 1H).

### Example 24: Preparation of Drug-Linker 24

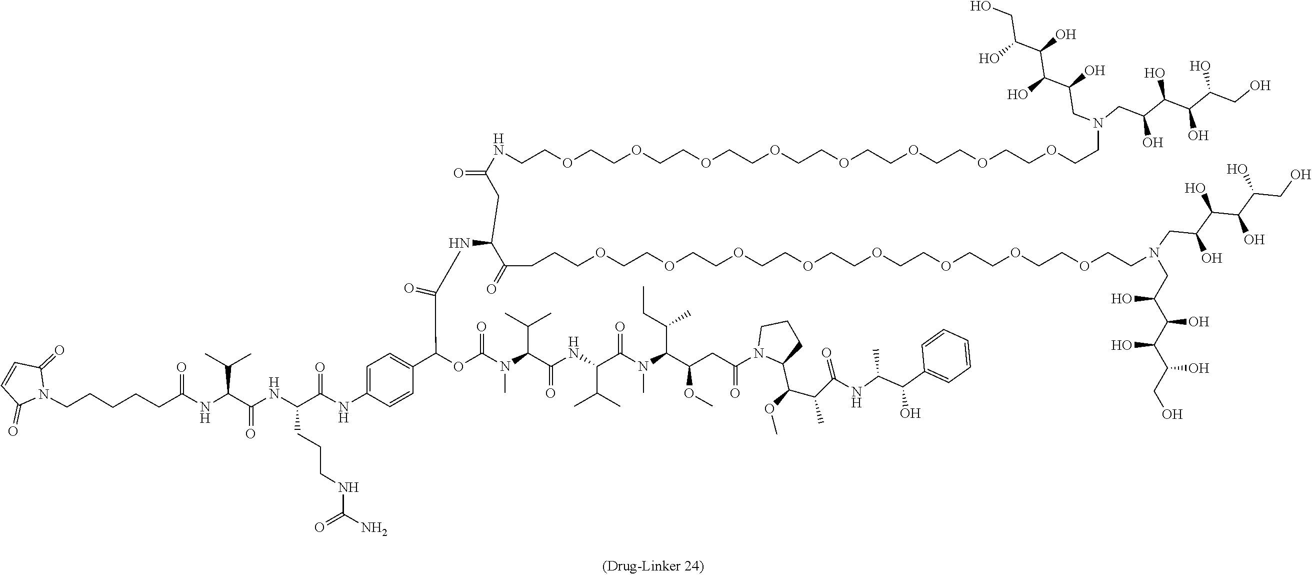 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@@H](CC(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)CCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

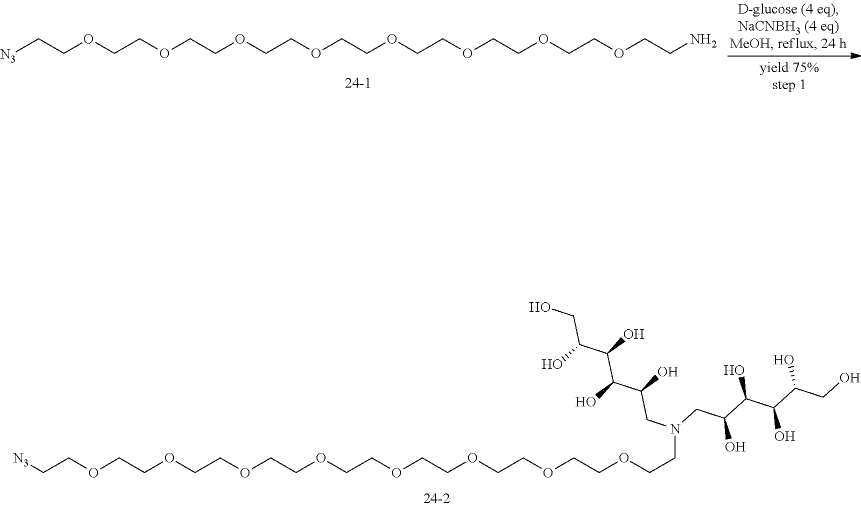 [SMILES: [N-]=[N+]=NCCOCCOCCOCCOCCOCCOCCOCCOCCN ; [N-]=[N+]=NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO]

To the solution of 24-1 (2.5 g, 5.701 mmol) in MeOH (20 mL) was added D-Glucose (4.11 g, 22.804 mmol) and NaBH3CN (1.385 mL, 22.804 mmol). The mixture was stirred at reflux for 24h to complete. Then the resulting solution concentrated to dryness and the residue was purified by reverse phase chromatography (C8 column, eluting with 0-45% methanol in water with 0.01% TFA) to afford the product 24-2 as yellow oil. ESI m/z: 767.5 (M+H)⁺.

### Step 2

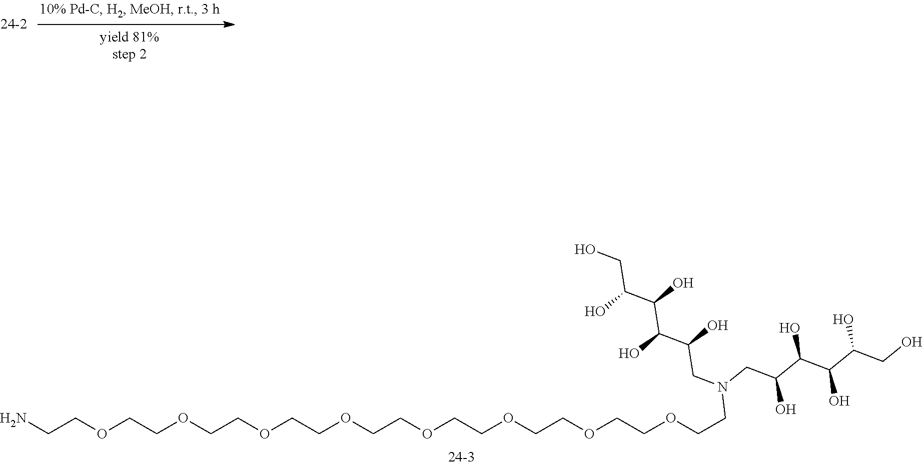 [SMILES: NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO]

To the solution of 24-2 (3.3 g, 4.303 mmol) in MeOH (20 mL) was added Pd/C (10% wt, 330 mg) under nitrogen and equipped with H₂ balloon. The reaction system was degassed and backfilled with hydrogen for three times and then stirred at room temperature under hydrogen atmosphere for 3h to complete. The resulting mixture was filtered to remove catalyst solid and the filtrate was concentrated, then purified by reverse phase chromatography (C8 column, eluting with 0-25% acetonitrile in water with 0.01% TFA) to afford the product 24-3 (2.6 g, 3.510 mmol, 81.50%) as colorless oil. ESI m/z: 371.3 (M/2+H)⁺, 741.4 (M+H)⁺.

### Step 3

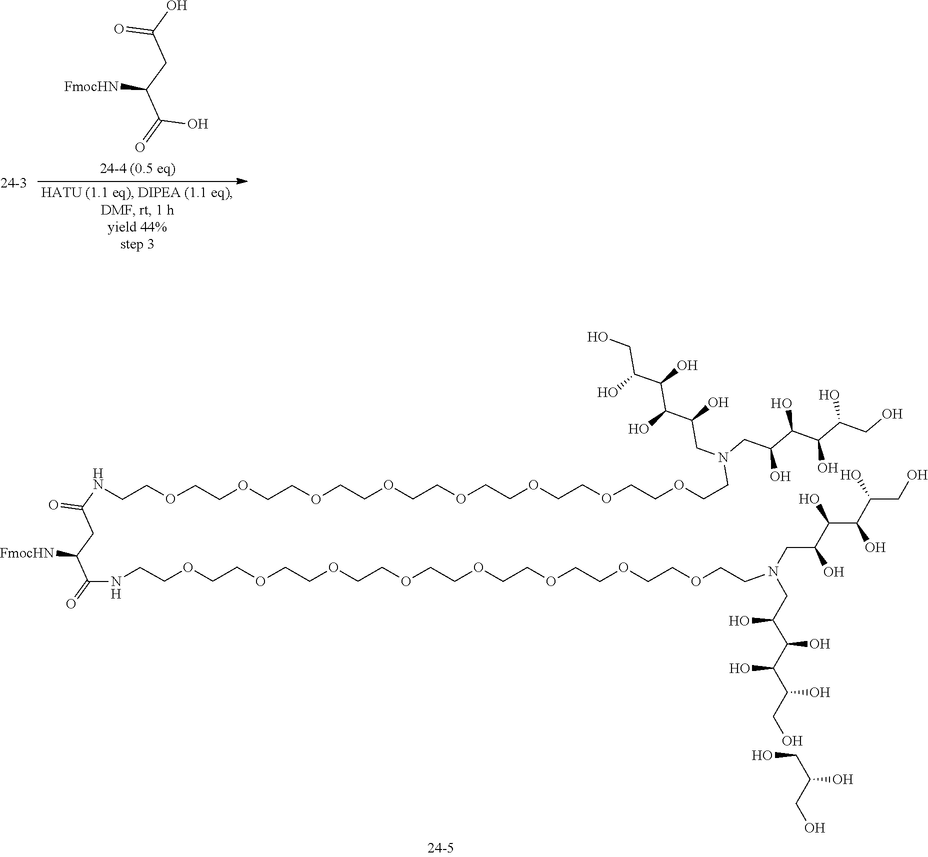

A solution of 24-4 (0.62 g, 1.755 mmol) in DMF (5 mL) was added HATU (1.47 g, 3.860 mmol) followed by DIPEA (0.50 g, 3.860 mmol). After stirring at room temperature for 15 min, the solution was added in dropwise manner into the solution of 24-3 (2.6 g, 3.510 mmol) in DMF (5 mL). After addition, the solution was stirred at room temperature for another 1h to complete. The completed solution was then purified directly by reverse phase chromatography (C8 column, eluting with 0-40% acetonitrile in water with 0.01% TFA) to afford the product 24-5 (1.4 g, 0.777 mmol, 44.30%) as colorless oil. ESI m/z: 601.0 (M/3+H)⁺, 901.0 (M/2+H)⁺.

### Step 4

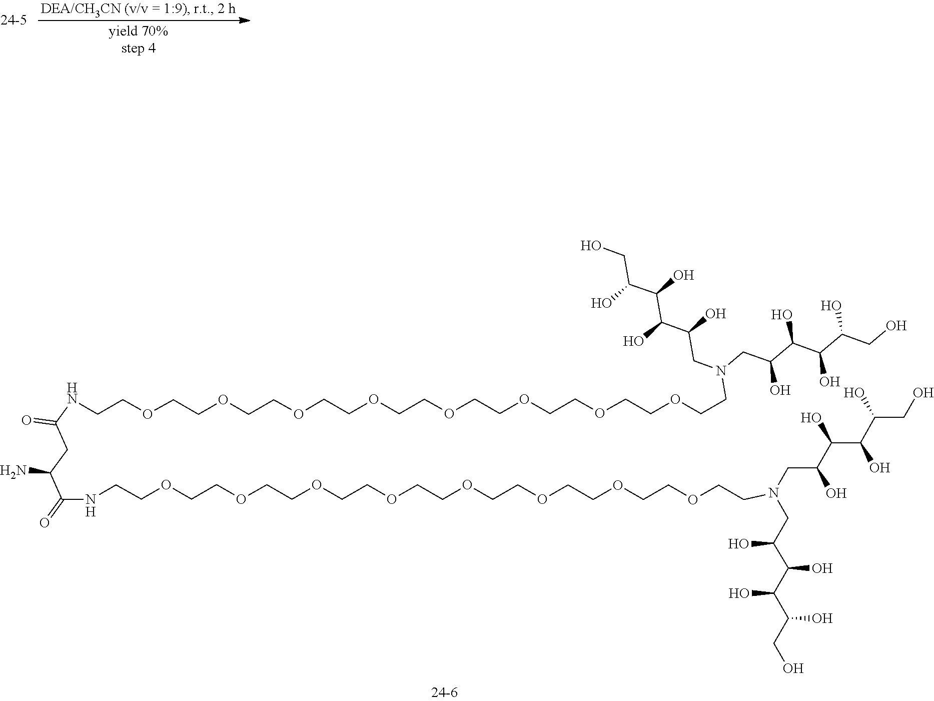

To the solution of 24-5 (1.4 g, 0.777 mmol) in MeCN (6 mL) was diethyl amine (0.7 mL, 8.930 mmol). The mixture was stirred at room temperature for 2h to achieve complete deprotection. Then the resulting solution was concentrated under reduced pressure to remove most of diethyl amine, and the residue was purified by reverse phase chromatography (C8 column, eluting with 0-20% acetonitrile in water with 0.01% TFA) to get desired fractions, which was freeze-dried to afford the product 24-6 (0.86 g, 0.545 mmol, 69.92%) as sticky colorless oil. ESI m/z: 526.9 (M/3+H)⁺, 789.9 (M/2+H)⁺. 1HNMR (400 MHz, DMSO-d6) δ 8.36 (t, J=5.6 Hz, 1H), 8.24 (t, J=5.6 Hz, 1H), 5.88-4.41 (m, 15H), 3.99-3.79 (m, 4H), 3.61-3.56 (m, 12H), 3.52-3.49 (m, 60H), 3.49-3.40 (m, 12H), 3.27-3.19 (m, 6H), 3.04-2.86 (m, 16H), 2.65-2.06 (m, 2H), 1.15 (t, J=7.2 Hz, 2H) ppm.

### Step 5

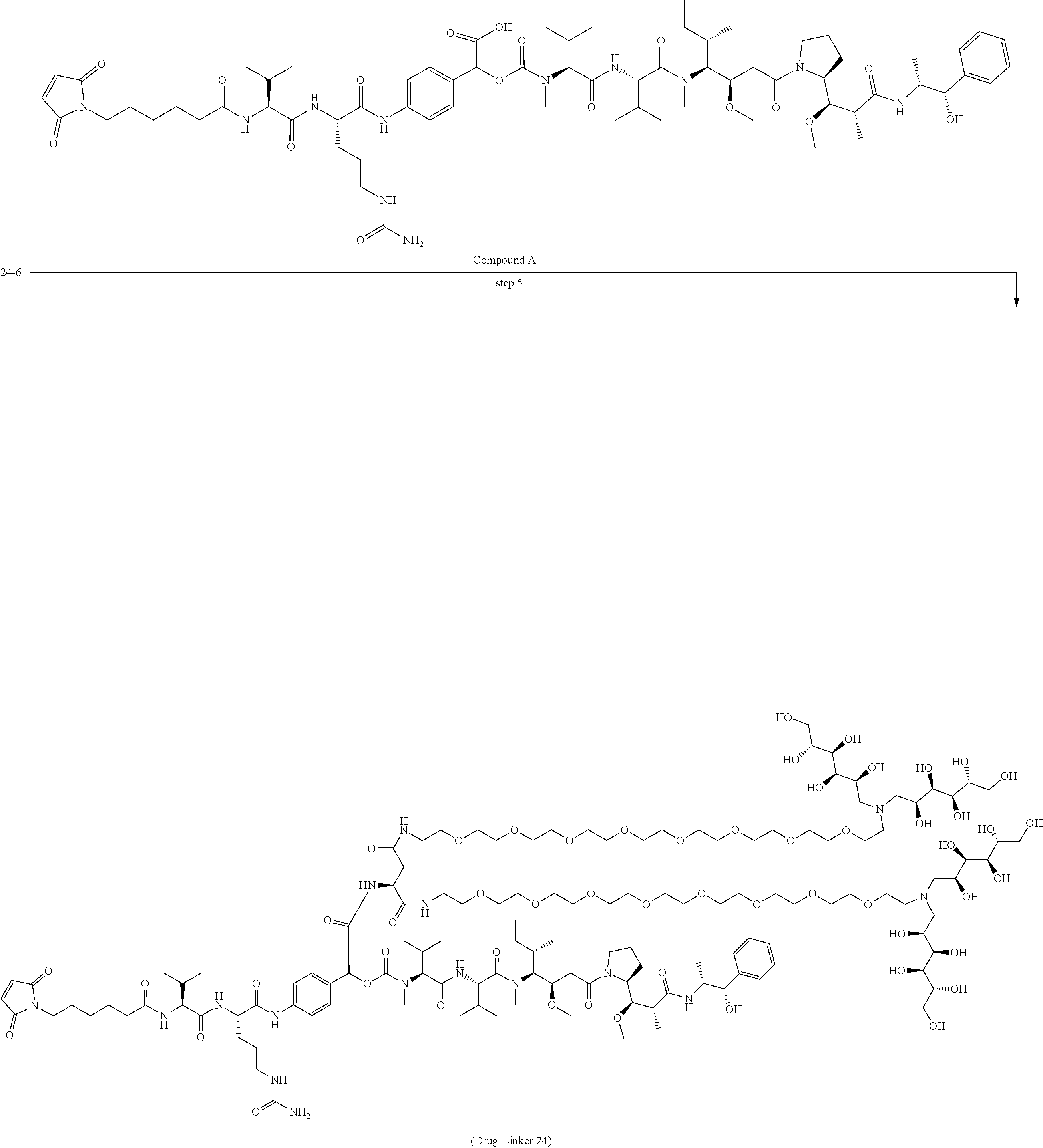

A solution of 24-6 (100 mg, 0.063 mmol), Compound A (86 mg, 0.063 mmol) and HATU (24 mg, 0.063 mnmol) in anhydrous DMF (4 mL) was stirred at room temperature for 5 min, then DIPEA (25 mg, 0.193 mmol) was added. The resulting solution was stirred for another 1 hr at r.t. until LCMS indicated complete reaction. Then the reaction solution was purified by prep. HPLC to give Drug-Linker 24 (40 mg, 0.014 mnmol, 21.73%) as a white solid. LCMS, m/z=1461.23 (M/2+H)⁺, ¹H NMR (400 MHz, D₂O) δ 7.54-7.48 (m, 2H), 7.45-7.37 (m, 2H), 7.37-7.26 (m, 5H), 6.73 (s, 2H), 4.40-4.38 (m, 2H), 4.19-4.15 (m, 6H), 3.85-3.83 (m, 5H), 3.78-3.74 (m, 10H), 3.71-3.66 (m, 13H), 3.63-3.61 (m, 49H), 3.58-3.56 (m, 17H), 3.49-3.43 (m, 14H), 3.39-3.35 (m, 4H), 3.30-3.29 (d, 3H), 3.24-3.23 (d, 4H), 3.06-3.00 (m, 4H), 2.77-2.64 (m, 2H), 2.22-2.21 (m, 2H), 1.99-1.97 (m, 2H), 1.75 (s, 5H), 1.53-1.45 (m, 10H), 1.24-1.08 (m, 9H), 1.02-1.01 (m, 2H), 0.92-0.75 (in, 28H) ppm.

### Example 25: Preparation of Drug-Linker 25

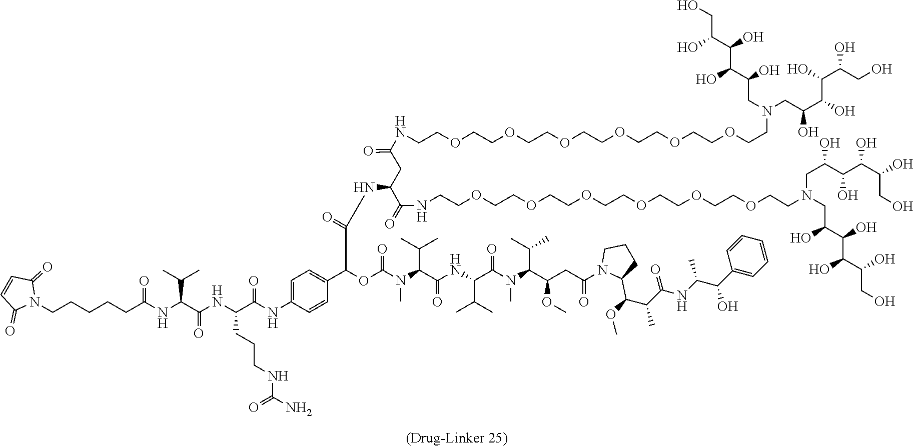

### Step 1

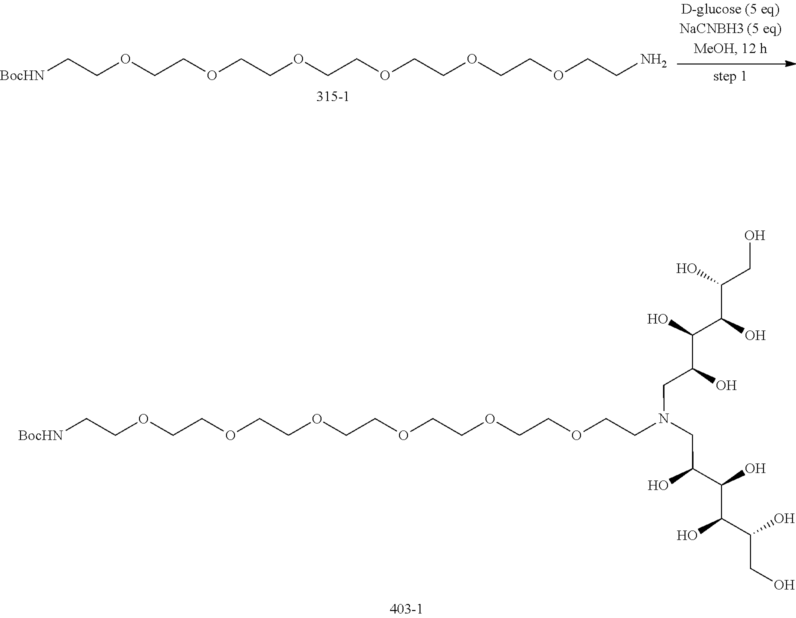 [SMILES: CC(C)(C)OC(=O)NCCOCCOCCOCCOCCOCCOCCN ; CC(C)(C)OC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO]

A solution of 315-1 (1.0 g, 2.355 mmol) and D-Glucose (2.1 g, 11.656 mmol) in anhydrous Methanol (40 mL) was heated at 50° C. for 30 min, then NaCNBH₃ (740 mg, 11.776 mmol) was added. The resulting solution was stirred for another 12 hr at 70° C. until indicated all starting amine was disappeared and the mass of desired product was detected. Then the reaction solution was concentrated and purified by reverse phase liquid chromatography to give 403-1 (730 mg, 0.970 mmol, 41.17%) as a white solid.

### Step 2

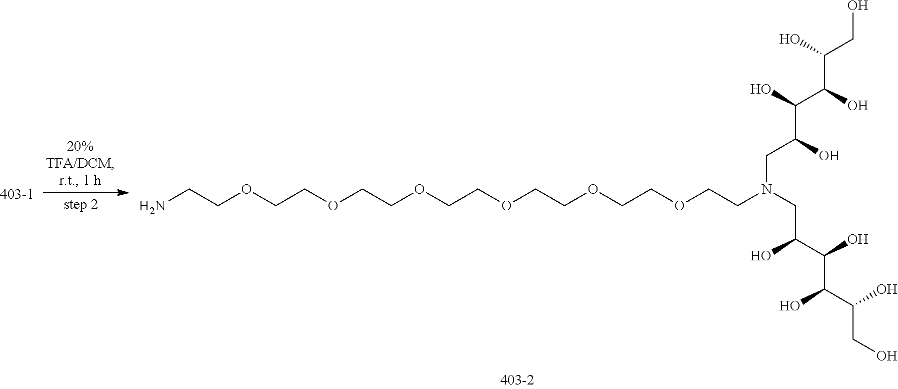 [SMILES: NCCOCCOCCOCCOCCOCCOCCN(C[C@H](O)[C@@H](O)[C@H](O)[C@H](O)CO)C[C@H](O)[C@@H](O)[C@H](O)[C@H](O)CO]

A solution of 403-1 (730 mg, 0.970 mmol) and TFA (2 mL) in anhydrous DCM (8 mL) was stirred at room temperature for 1 hr until LCMS indicated all starting amine was disappeared and desired product was detected. Then the solution was concentrated to dryness to 403-2 (610 mg, 0.935 mmol, 96.39%) as yellow oil, used as such in the next step.

### Step 3

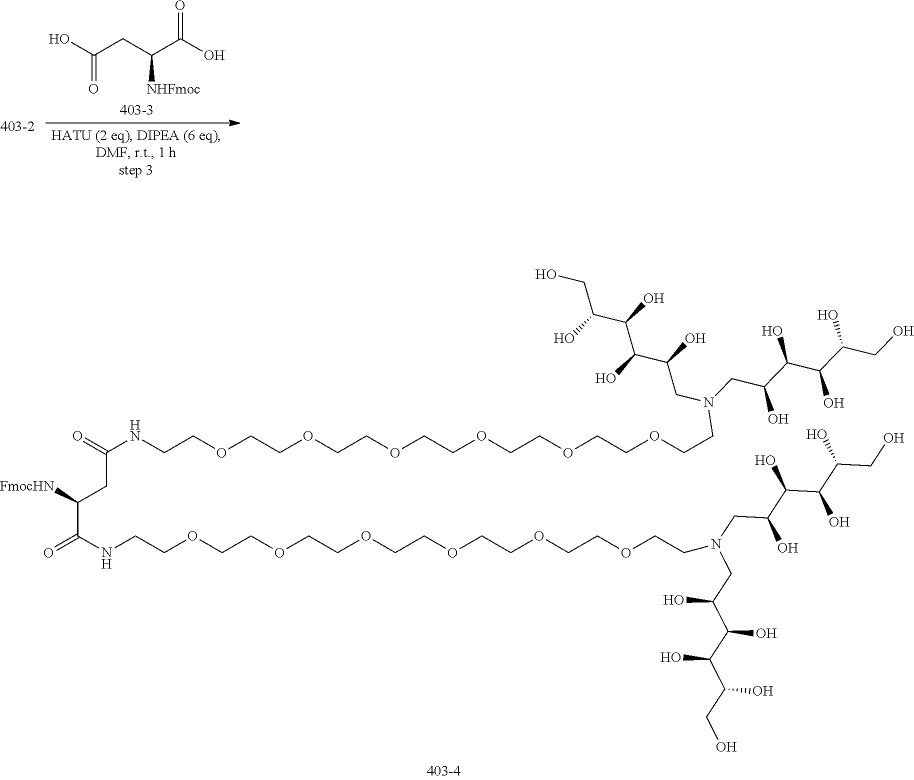

A solution of 403-2 (610 mg, 0.935 mmol), 403-3 (166 mg, 0.467 mmol) and HATU (355 mg, 0.934 mmol) in anhydrous DMF (5 mL) was stirred at room temperature for 5 min, then DIPEA (362 mg, 2.801 mmol) was added. The resulting solution was stirred for another 1 hr at r.t. until LCMS indicated complete reaction. The reaction solution was purified directly by reverse phase liquid chromatography to give 403-4 (410 mg, 0.252 mmol, 54.03%) as a white solid.

### Step 4

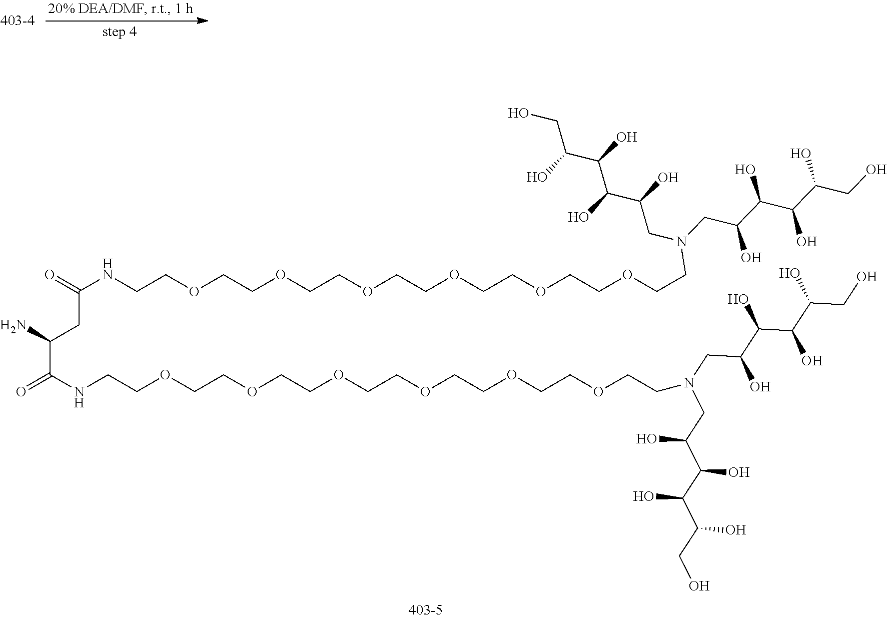 [SMILES: N[C@@H](CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO]

A solution of 403-4 (200 mg, 0.123 mmol) and DEA (1 mL) in anhydrous DMF (4 mL) was stirred at room temperature for 1 hr until LCMS indicated all starting amine was disappeared and desired product was detected. Then the solution was concentrated to dryness to 403-5 (167 mg, 0.119 mmol, 96.80%) as colorless oil, used as such in the next step.

### Step 5

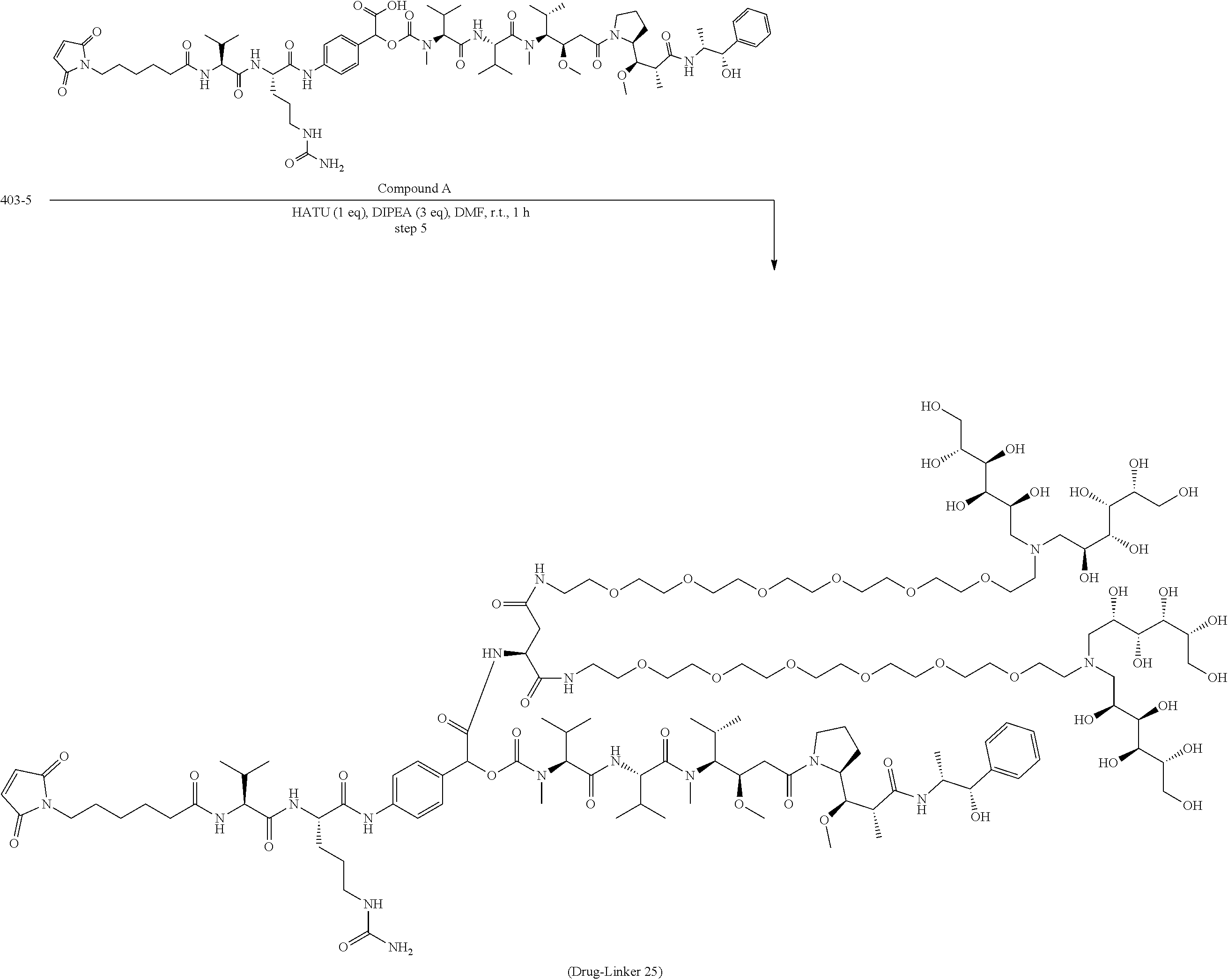

A solution of 403-5 (41 mg, 0.029 mmol), Compound A (40 mg, 0.029 mmol) and HATU (11 mg, 0.029 mmol) in anhydrous DMF (4 mL) was stirred at room temperature for 5 min, then DIPEA (11 mg, 0.085 mmol) was added. The resulting solution was stirred for another 1 hr at r.t. until LCMS indicated complete reaction. Then the reaction solution was purified by prep. HPLC to give Drug-Linker 25 (18 mg, 0.007 mmol, 22.61%) as a white solid. LCMS, m/z=1373.31 (M/2+H)⁺.

### Example 26: Preparation of Drug-Linker 26

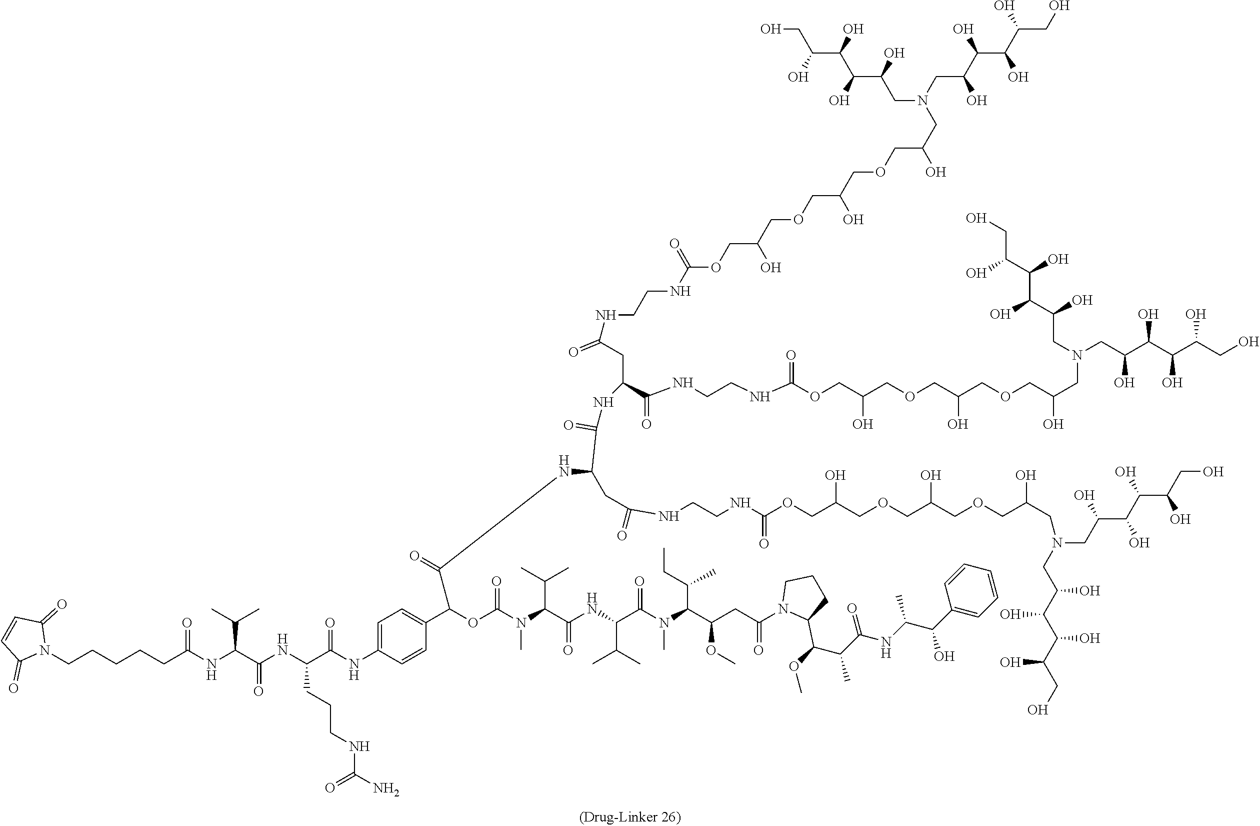

### Step 1

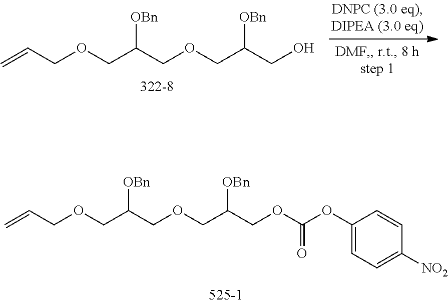 [SMILES: C=CCOCC(COCC(CO)OCc1ccccc1)OCc1ccccc1 ; C=CCOCC(COCC(COC(=O)Oc1ccc([N+](=O)[O-])cc1)OCc1ccccc1)OCc1ccccc1]

To a solution of 322-8 (600 mg, 1.554 mmol) in DMF (12 mL) was added DIPEA (602.4 mg 4.661 mmol), followed by 4,4′-dinitrodiphenyl carbonate (1.42 g, 4.661 mmol), then the resulting mixture was stirred at room temperature for 8 hrs until 322-8 was consumed detected by LCMS. The reaction solution was directly used in the next step without work-up procedure.

### Step 2

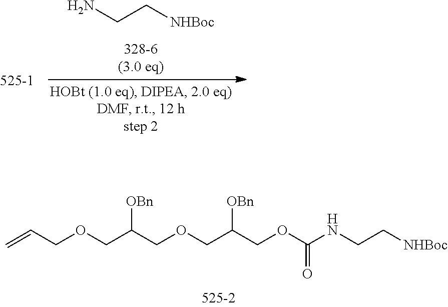 [SMILES: CC(C)(C)OC(=O)NCCN ; C=CCOCC(COCC(COC(=O)NCCNC(=O)OC(C)(C)C)OCc1ccccc1)OCc1ccccc1]

To the above reaction mixture was added HOBt (210 mg, 1.554 mmol), DIPEA (401.7 mg, 3.108 mmol) and 328-6 (746.5 mg, 4.662 mmol) successively, and the resulting mixture was stirred at room temperature for 6 hrs until 525-1 was consumed detected by LCMS. The reaction mixture was diluted with ethyl acetate (180 mL) and washed with saturated NaHCO₃ (aq, 45 mL×3), dried over anhydrous Na₂SO₄, filtered and concentrated to dryness under reduced pressure. The crude product was purified with column chromatography (silica, 0-80% ethyl acetate in petroleum ether) affording 525-2 (893 mg, 1.56 mmol, 100.4% over 2 steps) as a pale yellow oil.

### Step 3

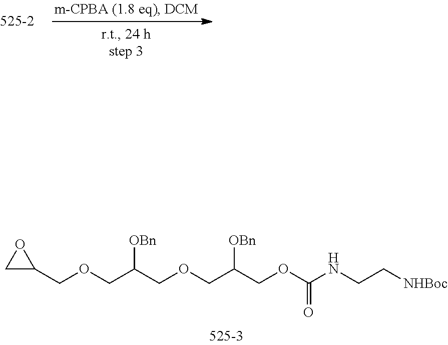 [SMILES: CC(C)(C)OC(=O)NCCNC(=O)OCC(COCC(COCC1CO1)OCc1ccccc1)OCc1ccccc1]

To a solution of 525-2 (890 mg, 1.555 mmol) in DCM (10 mL) was added a solution of m-CPBA (483 mg, 2.80 mmol) in DCM (10 mL) dropwise at room temperature. The resulting mixture was stirred at this temperature for 24 hours until 525-2 was consumed, and the reaction was quenched with saturated Na₂S₂O₃ (aq, 10 mL) and NaHCO₃ (aq., 10 mL). The reaction mixture was stirred for 30 mins, and then diluted with DCM (50 mL). The organic phase was washed with a mixture solution (20 mL×3) of saturated Na₂S₂O₃ and NaHCO₃ (aq, 1:1, V/V), dried over anhydrous Na₂SO₄, filtered and concentrated to dryness under reduced pressure to give the crude product, which was purified with column chromatography (silica, 0-100% ethyl acetate in petroleum ether) affording 525-3 (790 mg, 1.343 mmol, 86.4%) as a pale yellow oil. Purity=90%-95%.

### Step 4

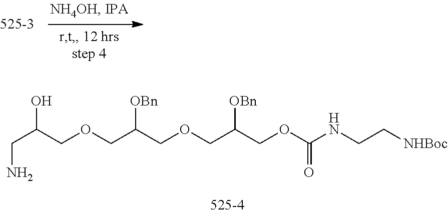 [SMILES: CC(C)(C)OC(=O)NCCNC(=O)OCC(COCC(COCC(O)CN)OCc1ccccc1)OCc1ccccc1]

To a solution of 525-3 (790 mg, 1.343 mmol) in isopropyl alcohol (110 mL) was added ammonia (110 mL) dropwise at room temperature, and the resulting mixture was stirred at this temperature for 12 hours until 525-3 was consumed. The reaction mixture was concentrated to dryness under reduced pressure to afford 525-4 (801.2 mg, 1.323 mmol, 98.6%) as a yellow oil, which was used in the next step without further purification. Purity=90%-95%.

### Step 5

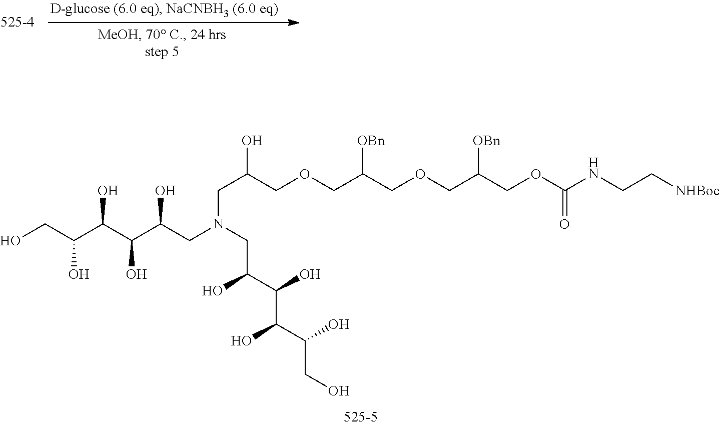 [SMILES: CC(C)(C)OC(=O)NCCNC(=O)OCC(COCC(COCC(O)CN(C[C@H](O)[C@@H](O)[C@H](O)[C@H](O)CO)C[C@H](O)[C@@H](O)[C@H](O)[C@H](O)CO)OCc1ccccc1)OCc1ccccc1]

A mixture of 525-4 (801.2 mg, 1.324 mmol), D-glucose (1.43 g, 7.937 mmol) and NaCNBH₃ (499.2 mg, 7.94 mmol) in anhydrous MeOH (21 mL) was stirred at 70° C. for 24 hrs until most of 525-4 was consumed and 525-5 was detected by LCMS. The reaction mixture was cooled down to room temperature, filtered and concentrated under reduced pressure to give the crude product, which was purified by reverse phase liquid chromatography to give 525-5 (1.2 g, 1.29 mmol, 97.1%) as a colorless oil. Purity=85%-90%.

### Step 6

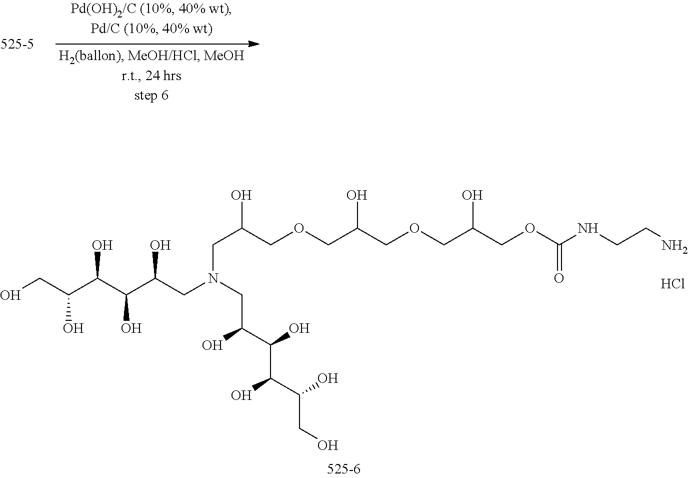 [SMILES: NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO ; Cl]

A mixture of 525-5 (1.2 g, 1.286 mmol), Pd(OH)₂/C (10%, 250 mg) and Pd/C (10%, 250 mg) in HCl/MeOH (4M, 25 mL), MeOH (25 mL) was stirred under hydrogen atmosphere (ballon) for 24 h at room temperature until 525-5 was completely converted into 525-6. The reaction mixture was filtered through a celite pad, and the filtrate was concentrated to dryness under reduced pressure to afford 525-6 (886 mg, 1.285 mmol, 100.0%) as an off-white solid, which was used in the next step without further purification.

### Step 7

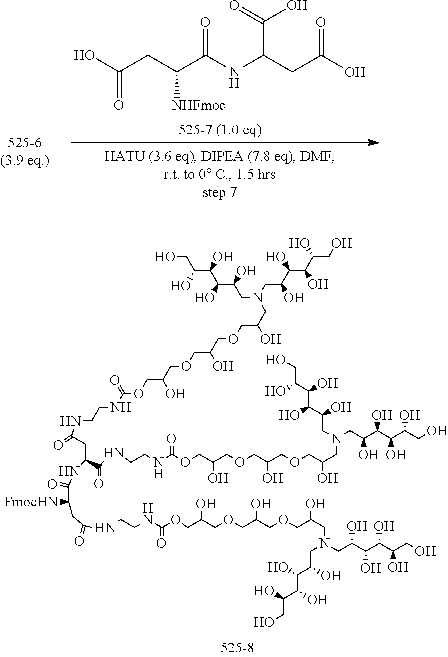

A solution of 525-7 (104.9 mg, 0.223 mmol) and HATU (305.4 mg, 0.803 mmol) in anhydrous DMF (3 mL) was stirred at room temperature for 15 mins, then it was stirred in an ice bath. A solution of 525-6 (600 mg, 0.870 mmol) in anhydrous DMF (3 mL) was added dropwise, followed by DIPEA (225 mg, 1.74 mmol). The resulting mixture was stirred in the ice bath for 1 h until most of 526-7 was consumed. The reaction mixture was purified by reverse phase liquid chromatography to give 525-8 (269.8 mg, 0.113 mmol, 50.9%) as a white solid. Purity=90%-95%. ¹H NMR (400 MHz, DMSO-d₆) δ 7.87 (d, J=7.6 Hz, 2H), 7.64 (d, J=7.2 Hz, 2H), 7.47 (t, J=7.6 Hz, 2H), 7.42-7.32 (m, 2H), 4.65-4.57 (m, 1H), 4.53-4.36 (m, 3H), 4.33-4.13 (m, 10H), 4.08-3.87 (m, 12H), 3.84-3.69 (m, 18H), 3.67-3.35 (m, 53H), 3.30-3.07 (m, 12H), 2.77-2.32 (m, 4H).

### Step 8

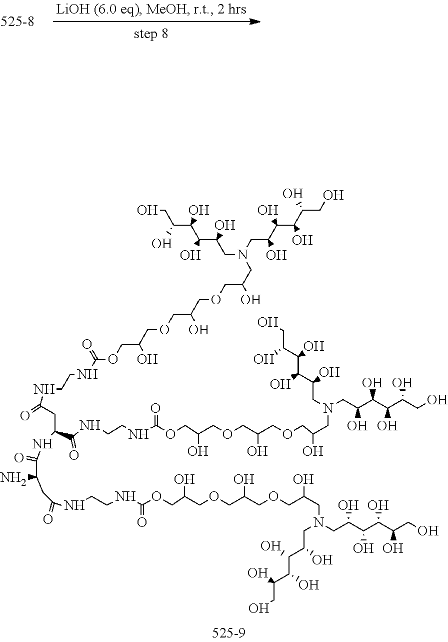 [SMILES: N[C@H](CC(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)N[C@@H](CC(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO]

To a solution of 525-8 (100 mg, 0.0421 mmol) in MeOH (3 mL) and H₂O (1 mL) was added LiOH·H₂O (10.6 mg, 0.252 mmol), and the mixture was stirred at room temperature for 2 hrs until 525-8 was consumed. The reaction solution was neutralized with 1N HCl to pH=7, and concentrated under reduced pressure to give a crude product, which was dissolved in H₂O (15 mL) and washed with hexane (10 mL×3). The aqueous phase was concentrated to dryness under reduced pressure to afford 525-9 (74.6 mg, 0.0346 mmol, 82.3%) as a colorless oil, which was used in the next step without further purification.

### Step 9

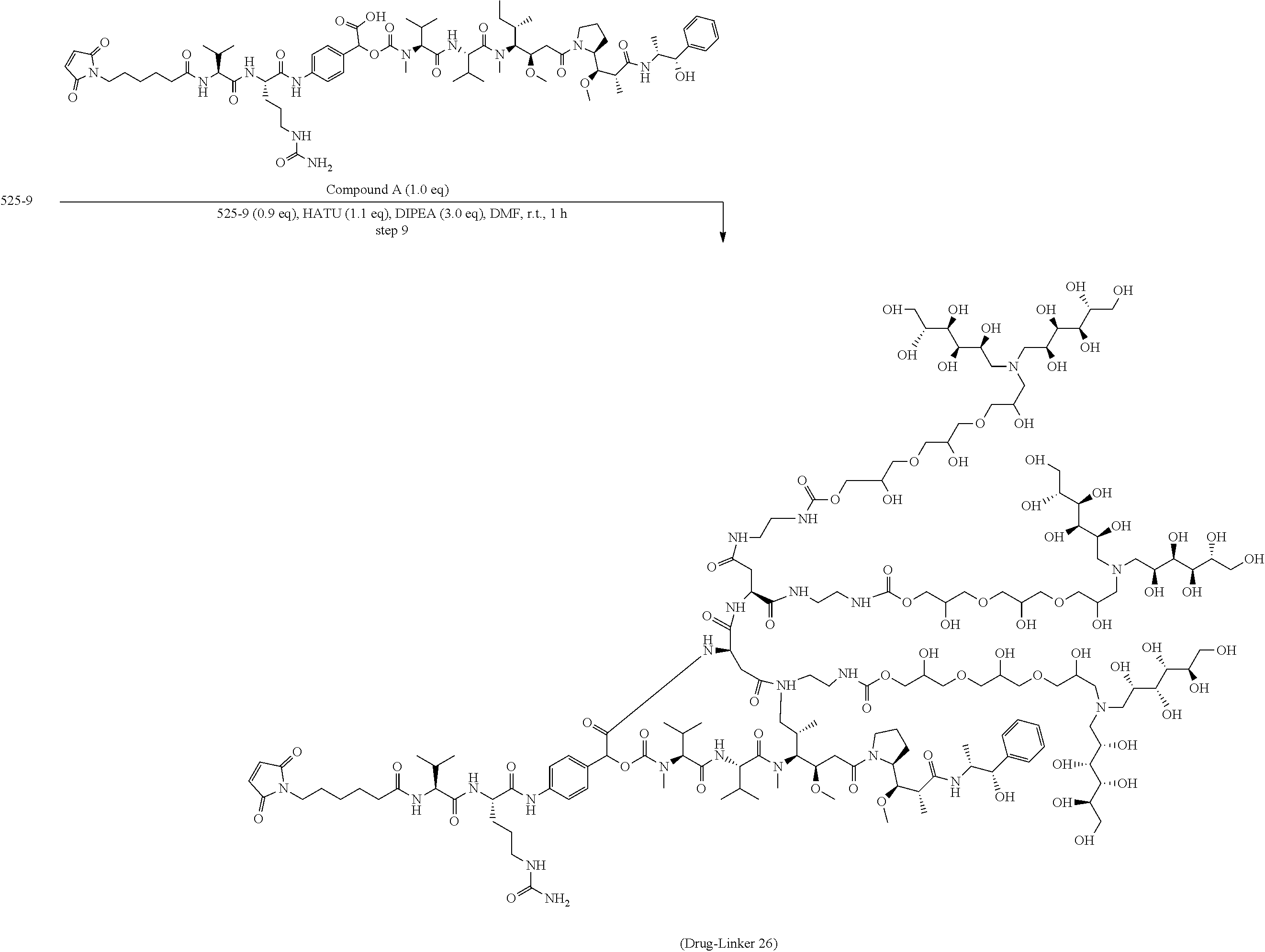

A solution of 525-9 (70.0 mg, 0.0325 mmol), Compound A(49 mg, 0.0360 mmol), HATU (15.1 mg, 0.0397 mmol) and DIPEA (14.0 mg, 0.108 mmol) in anhydrous DMF (4 mL) was stirred at room temperature for 1 h until Compound A was consumed. Then the reaction solution was purified by prep-HPLC to give Drug-Linker 26 (23.5 mg, 0.00672 mmol, 20.7%) as a white solid. LCMS, m/z=1749.66 (M/2+H)⁺, m/z=1166.72 (M/2+H)⁺. ¹H NMR (400 MHz, DMSO-d₆) δ 7.61-7.40 (m, 4H), 7.39-7.26 (m, 4H), 7.25-7.13 (m, 1H), 6.77 (s, 2H), 5.98 (t, J=13.6 Hz, 1H), 4.75-4.52 (m, 7H), 4.49-4.38 (m, 2H), 4.37-3.90 (m, 25H), 3.87-3.38 (m, 73H), 3.37-2.87 (m, 28H), 2.87-2.08 (m, 12H), 2.07-1.95 (m, 2H), 1.93-1.65 (m, 5H), 1.68-1.41 (m, 8H), 1.37-1.11 (m, 8H), 1.09-1.02 (m, 2H), 1.00-0.75 (m, 20H), 0.74-0.67 (m, 1H), 0.57-0.48 (m, 1H), 0.33 (d, J=6.4 Hz, 1H).

### Example 27: Preparation of Drug-Linker 27

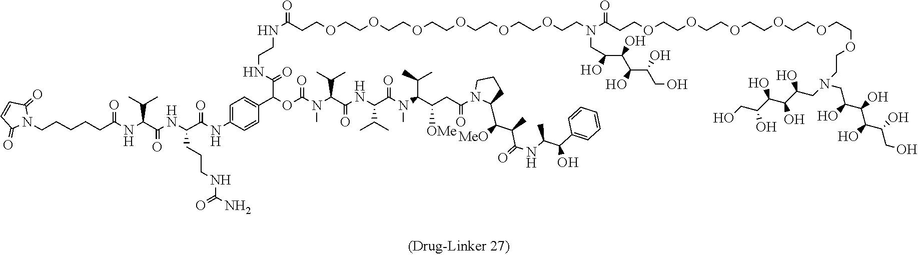

### Step 1

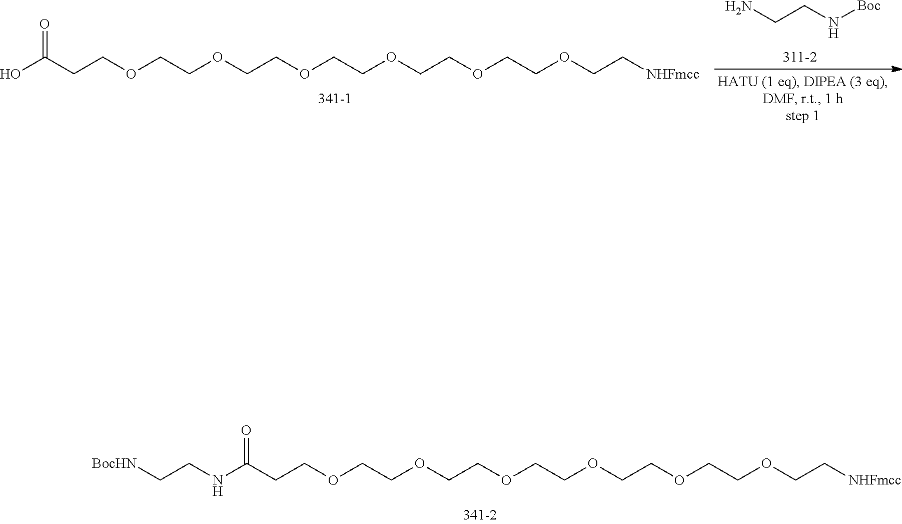

A solution of 341-1 (1 g, 1.737 mmol), 311-2 (278 mg, 1.735 mmol) and HATU (661 mg, 1.738 mmol) in anhydrous DMF (10 mL) was stirred at room temperature for 5 min, then DIPEA (674 mg, 5.215 mmol) was added. The resulting solution was stirred for another 1 hr at r.t. until LCMS indicated complete reaction. The reaction solution was purified directly by reverse phase liquid chromatography to give 341-2 (950 mg, 1.323 mmol, 76.17%) as a white solid. purity=90%-95%.

### Step 2

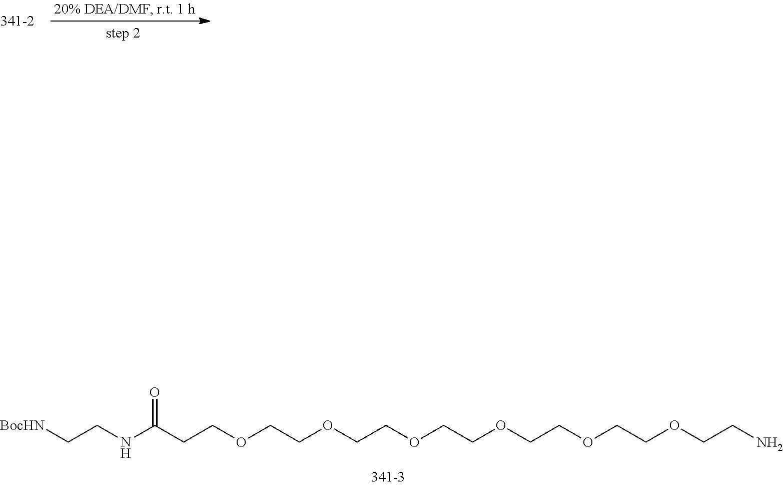 [SMILES: CC(C)(C)OC(=O)NCCNC(=O)CCOCCOCCOCCOCCOCCOCCN]

A solution of 341-2 (950 mg, 1.323 mmol) and DEA (2 mL) in anhydrous DMF (8 mL) was stirred at room temperature for 1 hr until LCMS indicated all starting amine was disappeared and desired product was detected. Then the solution was concentrated to dryness to 341-3 (610 mg, 1.231 mmol, 93.05%) as colorless oil, used as such in the next step. purity=90%-95%.

### Step 3

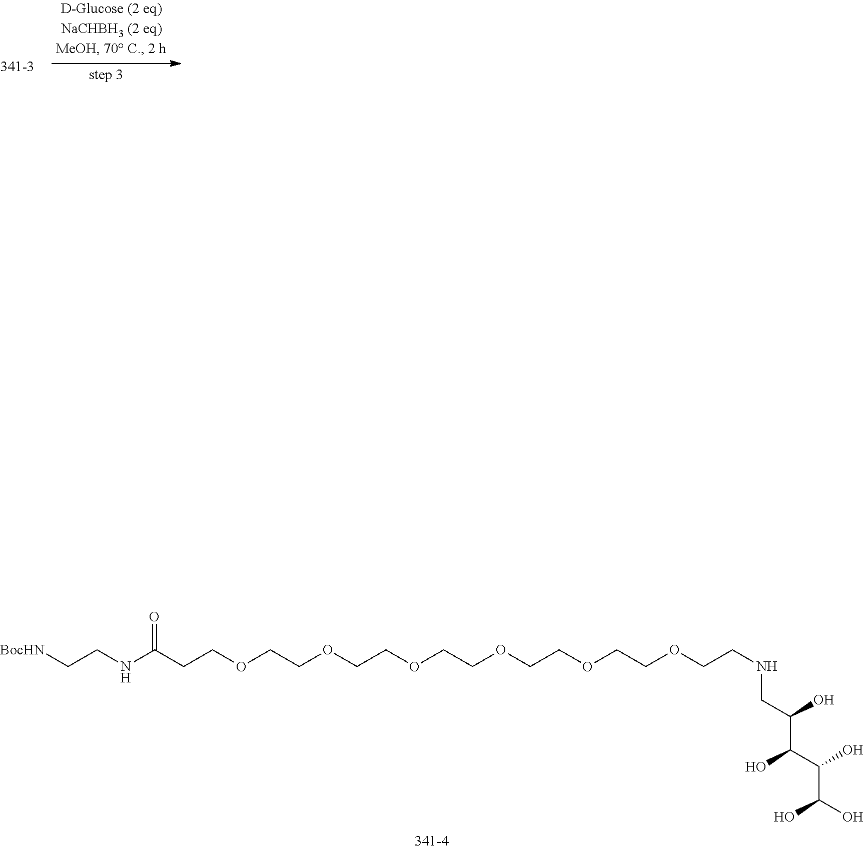

A solution of 341-3 (610 mg, 1.231 mmol) and D-Glucose (444 mg, 2.464 mmol) in anhydrous Methanol (50 mL) was heated at 50° C. for 30 min, then NaCNBH₃ (155 mg, 2.467 mmol) was added. The resulting solution was stirred for another 2 hr at 70° C. until indicated all starting amine was disappeared and the mass of desired product was detected. Then the reaction solution was concentrated and purified by reverse phase liquid chromatography to give 341-4 (340 mg, 0.515 mmol, 41.84%) as a white solid. purity=90%-95%.

### Step 4

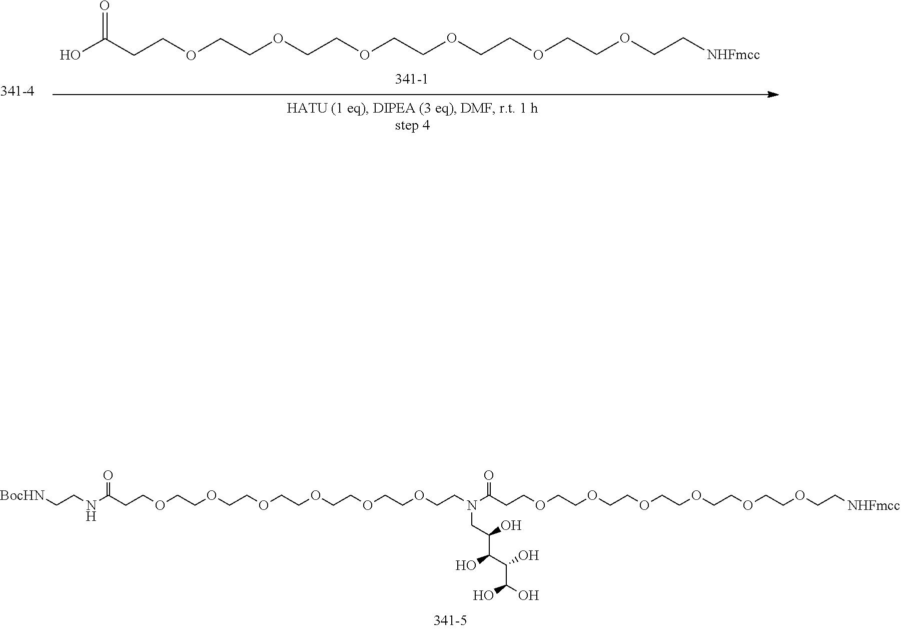

A solution of 341-4 (340 mg, 0.515 mmol), 341-1(297 mg, 0.516 mmol) and HATU (196 mg, 0.515 mmol) in anhydrous DMF (10 mL) was stirred at room temperature for 5 min, then DIPEA (200 mg, 1.548 mmol) was added. The resulting solution was stirred for another 1 hr at r.t. until LCMS indicated complete reaction. The reaction solution was purified directly by reverse phase liquid chromatography to give 341-5 (475 mg, 0.390 mmol, 75.73%) as a white solid. purity=90%-95%.

### Step 5

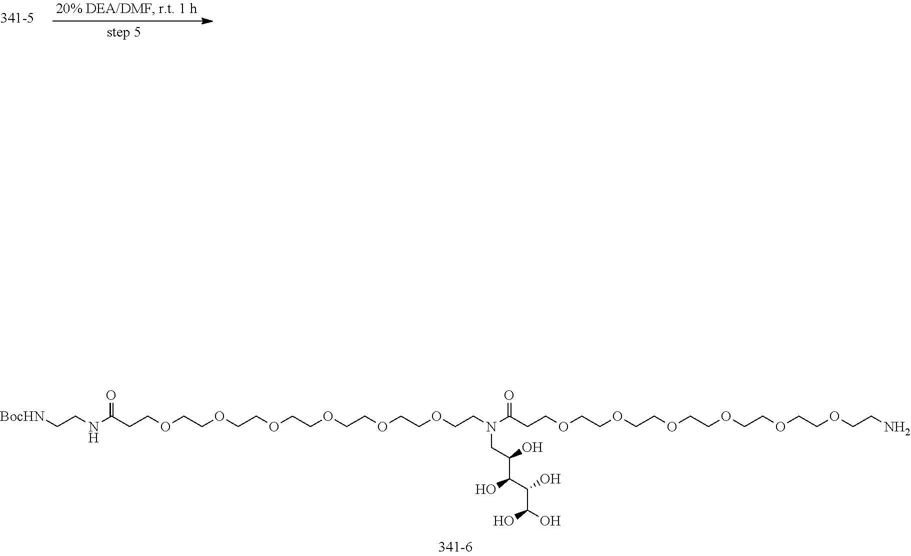

A solution of 341-5 (475 mg, 0.390 mmol) and DEA (2 mL) in anhydrous DMF (8 mL) was stirred at room temperature for 1 hr until LCMS indicated all starting amine was disappeared and desired product was detected. Then the solution was concentrated to dryness to 341-6 (372 mg, 0.374 mmol, 95.90%) as colorless oil, used as such in the next step. purity=90%-95%.

### Step 6

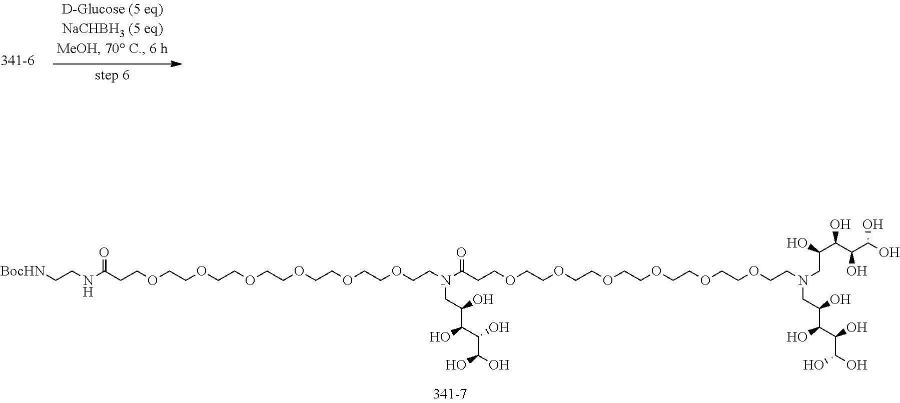

A solution of 341-6 (372 mg, 0.374 mmol) and D-Glucose (337 mg, 1.871 mmol) in anhydrous Methanol (50 mL) was heated at 50° C. for 30 min, then NaCNBH₃ (118 mg, 1.878 mmol) was added. The resulting solution was stirred for another 6 hr at 70° C. until indicated all starting amine was disappeared and the mass of desired product was detected. Then the reaction solution was concentrated and purified by reverse phase liquid chromatography to give 341-7 (406 mg, 0.307 mmol, 82.09%) as a white solid. purity=90%-95%.

### Step 7

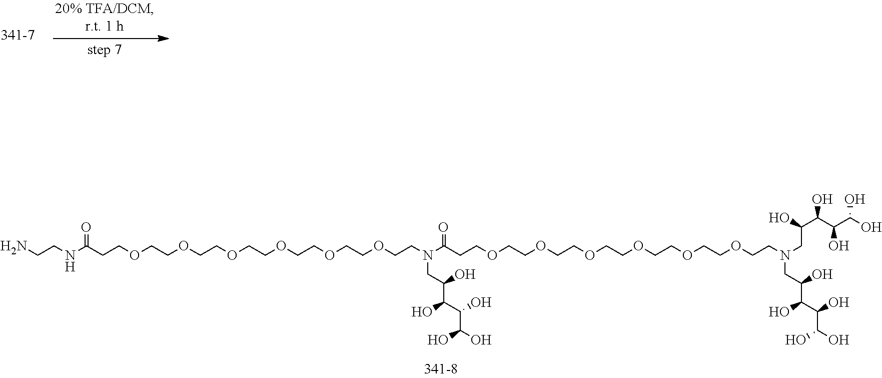

A solution of 341-7 (100 mg, 0.076 mmol) and TFA (2 mL) in anhydrous DCM (8 mL) was stirred at room temperature for 1 hr until LCMS indicated all starting amine was disappeared and desired product was detected. Then the solution was concentrated to dryness to 341-8 (85 mg, 0.069 mmol, 90.79%) as yellow oil, used as such in the next step. purity=90%-95%.

### Step 8

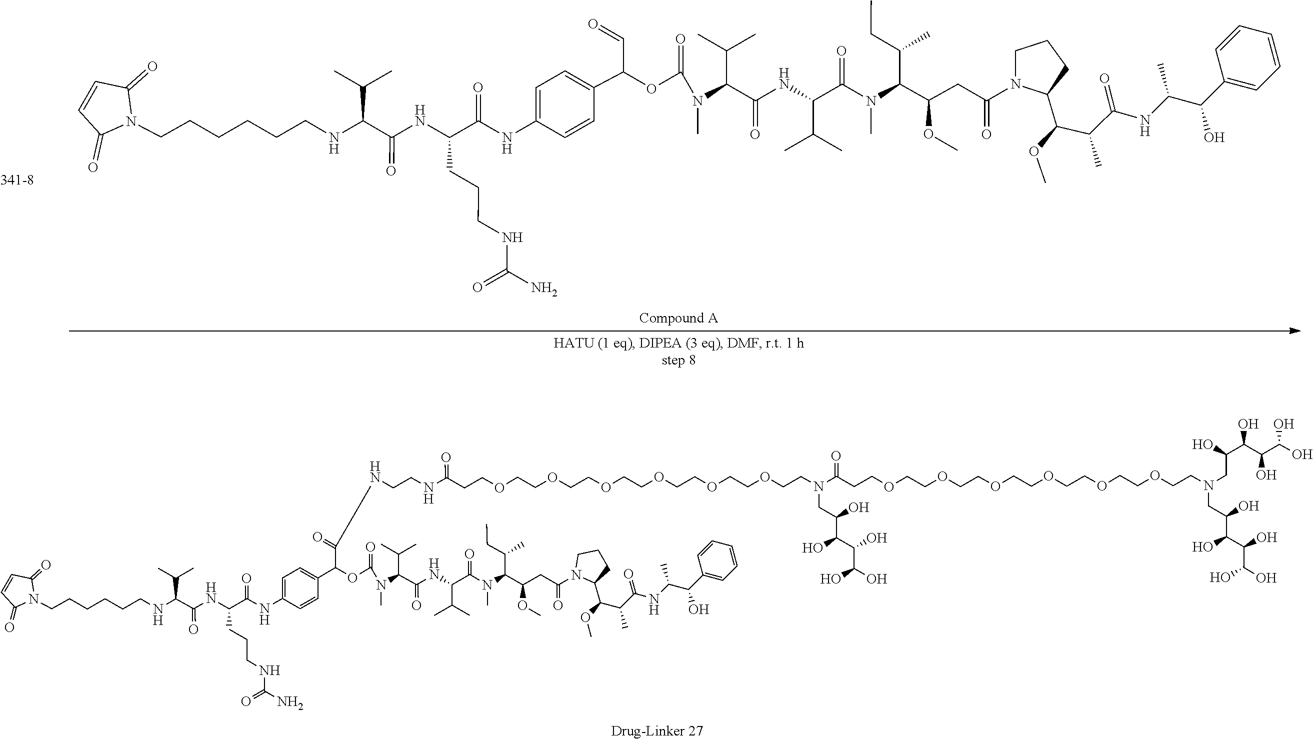

A solution of 341-8 (30 mg, 0.025 mmol), Compound A (33 mg, 0.025 mmol) and HATU (9 mg, 0.025 mmol) in anhydrous DMF (4 mL) was stirred at room temperature for 5 min, then DIPEA (10 mg, 0.074 mmol) was added. The resulting solution was stirred for another 1 hr at r.t. until LCMS indicated complete reaction. Then the reaction solution was purified by prep. HPLC to give Drug-Linker 27 (15 mg, 0.006 mmol, 24.01%) as a white solid. LCMS, m/z=856.38 (M/3+H)⁺.

### Example 28: Preparation of Drug-Linker 28

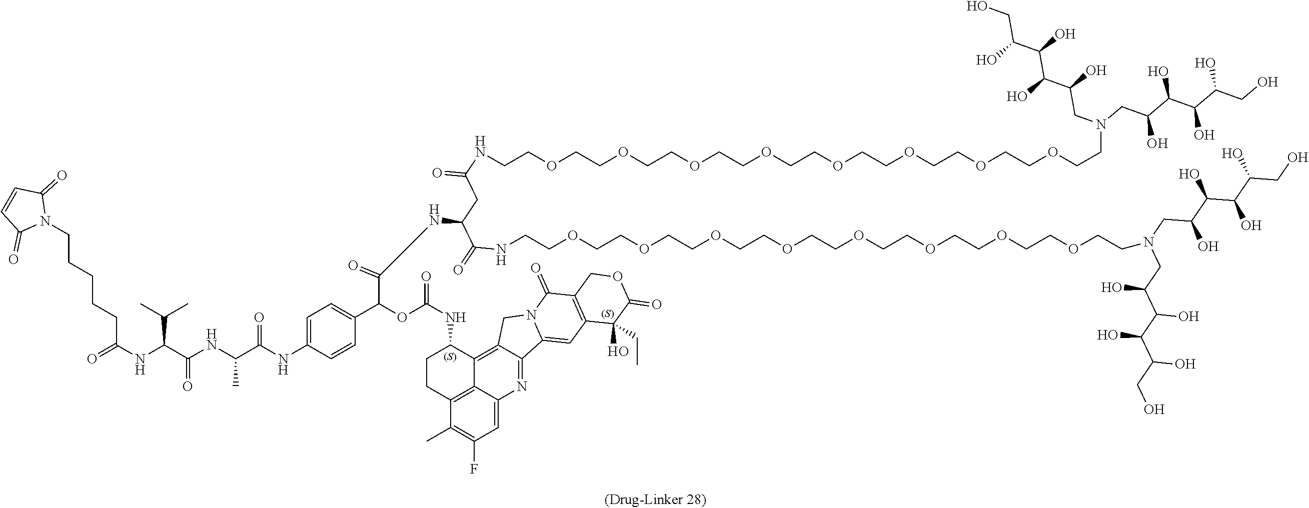

### Step 1

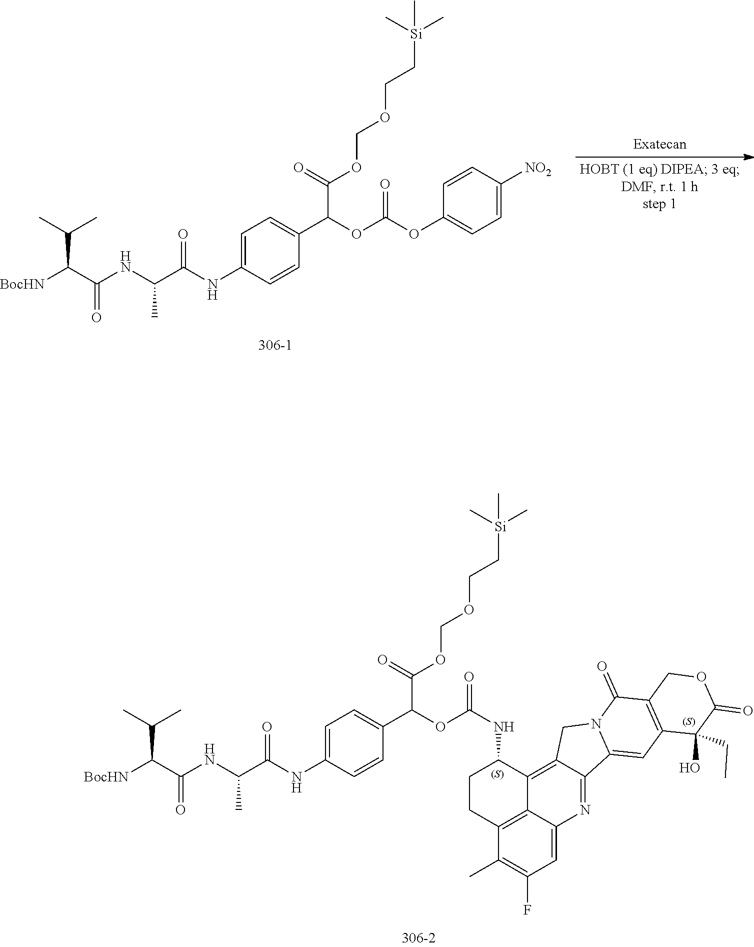

A solution of 306-1 (900 mg, 1.2 mmol), Exatecan (653 mg, 1.2 mmol) and HOBT (166 mg, 1.2 mmol) in anhydrous DMF (6 mL) was stirred at room temperature, then DIPEA (476 mg, 3.7 mmol) was added. The resulting solution was stirred for another 2 hr at r.t. until LCMS indicated all starting amine was disappeared and desired product was detected. Then the solution was concentrated to dryness, used as such in the next step. purity=80%-90%.

### Step 2

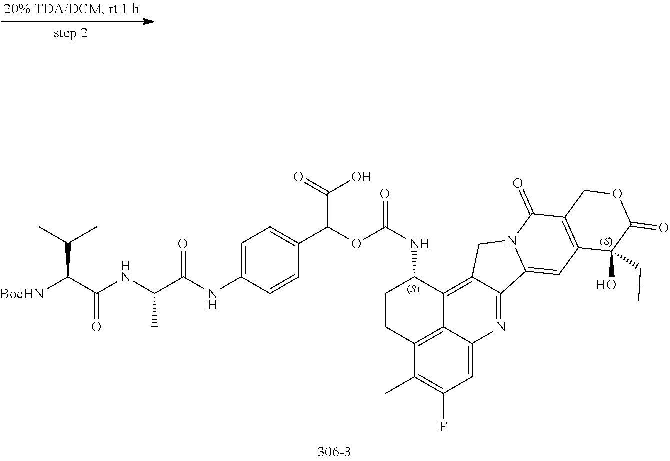

A solution of 306-2 and TFA (1 mL) in anhydrous DCM (5 mL) was stirred at room temperature for 1 hr until LCMS indicated all starting amine was disappeared and desired product was detected. Then the solution was concentrated and purified by reverse phase liquid chromatography to give 306-3 (120 mg, 0.13 mmol) as a gray solid, purity=70%-80%.

### Step 3

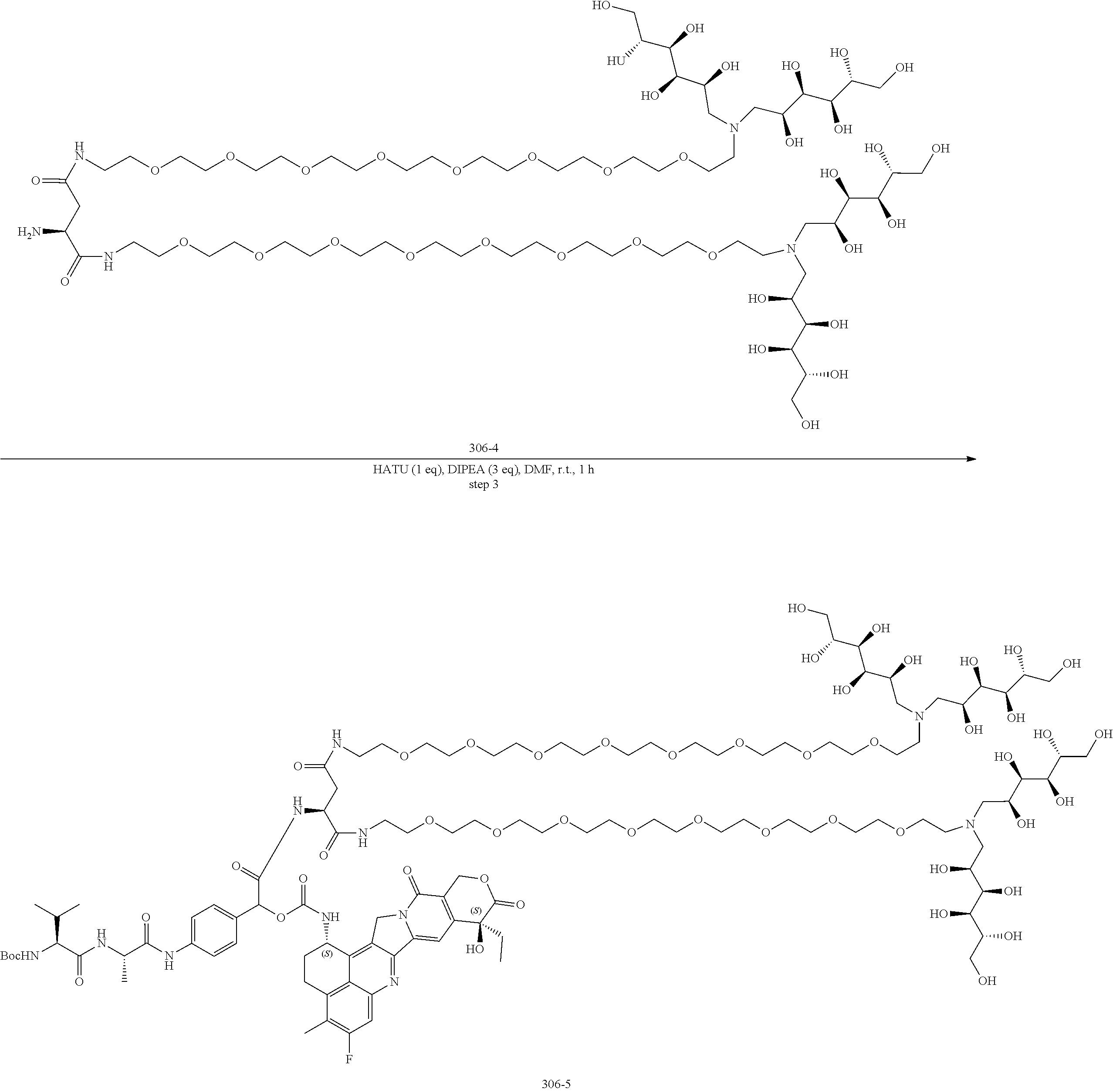

A solution of 306-3 (100 mg, 0.1 mmol), 306-4 (176 mg, 0.1 mmol) and HATU (42 mg, 0.1 mmol) in anhydrous DMF (3 mL) was stirred at room temperature for 5 min, then DIPEA (43 mg, 0.3 mmol) was added. The resulting solution was stirred for another 1 hr at r.t. until LCMS indicated complete reaction. The reaction solution was purified directly by reverse phase liquid chromatography to give 306-5 (60 mg, 0.024 mmol) as a gray solid, purity=90%-95%.

### Step 4

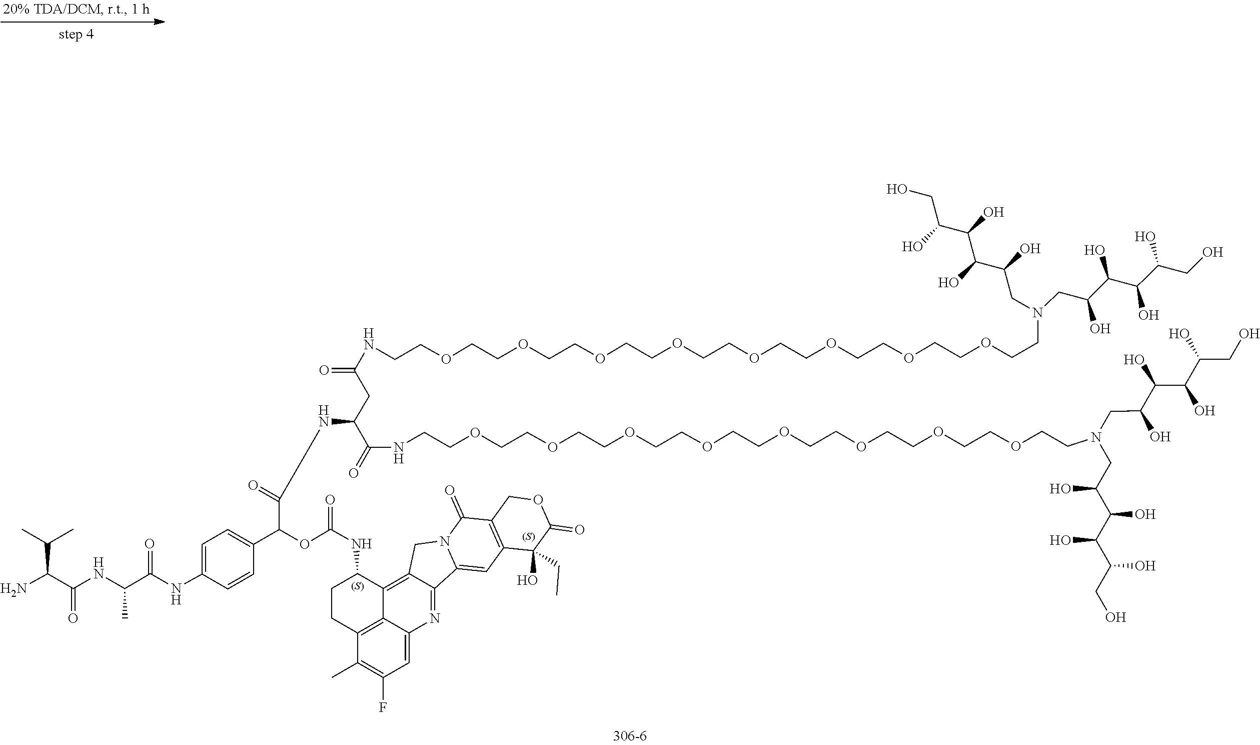

A solution of 306-5 (60 mg, 0.024 mmol) and TFA (1 mL) in anhydrous DCM (4 mL) was stirred at room temperature for 1 hr until LCMS indicated all starting amine was disappeared and desired product was detected. Then the solution was concentrated to dryness to 306-6 (55 mg, 0.024 mmol) as yellow oil, used as such in the next step. purity=85%-90%.

### Step 5

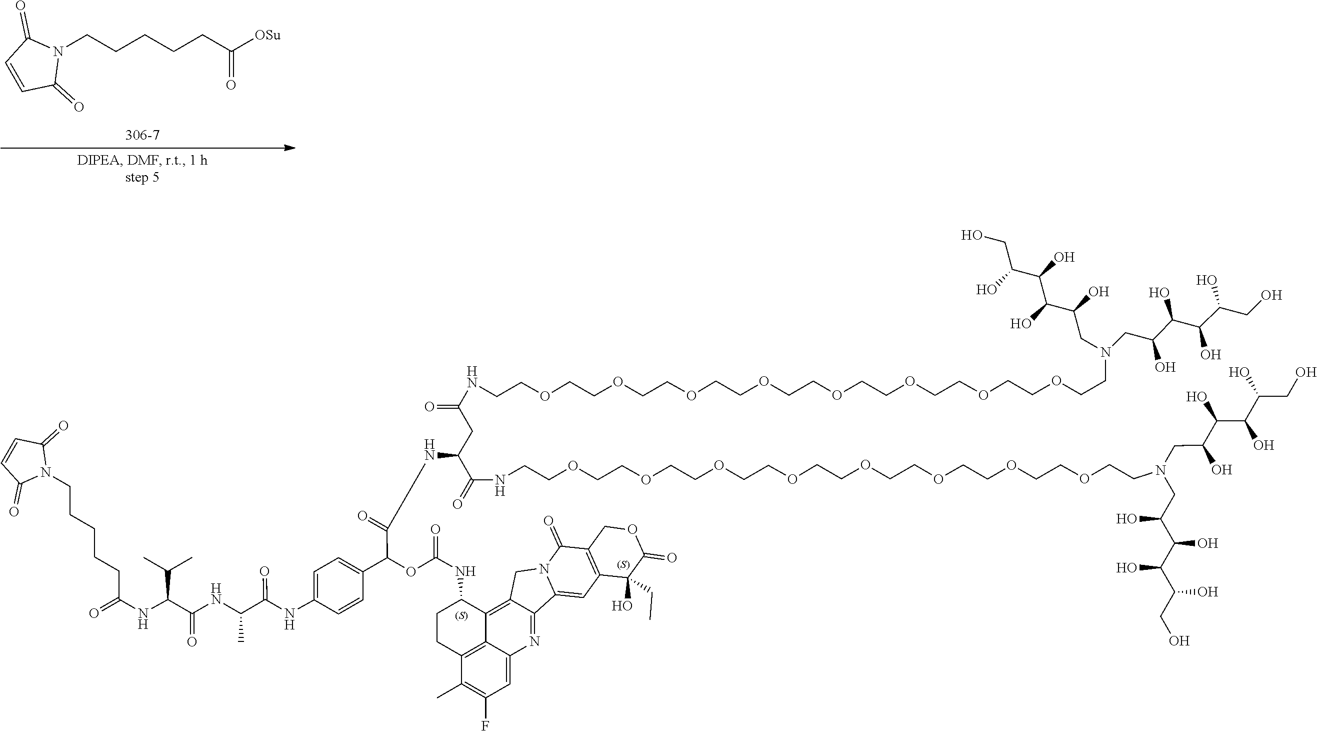

A solution of 306-6 (55 mg, 0.024 mmol) in MeCN (2 mL) and H₂O (2 mL) was stirred at room temperature for 5 min, then a solution of 306-7 (11 mg, 0.036 mmol) in anhydrous MeCN (1 mL) was added dropwise by syringe over 5 min. The resulting solution was stirred for another 1 hr at r.t. until indicated all starting amine was disappeared and the mass of desired product was detected. Then the reaction solution was purified by prep. HPLC to give LD306 (10 mg) as yellow solid. LCMS, m/z=1277.38 (M/2+H)⁺.

### Example 29: Preparation of Drug-Linker 29

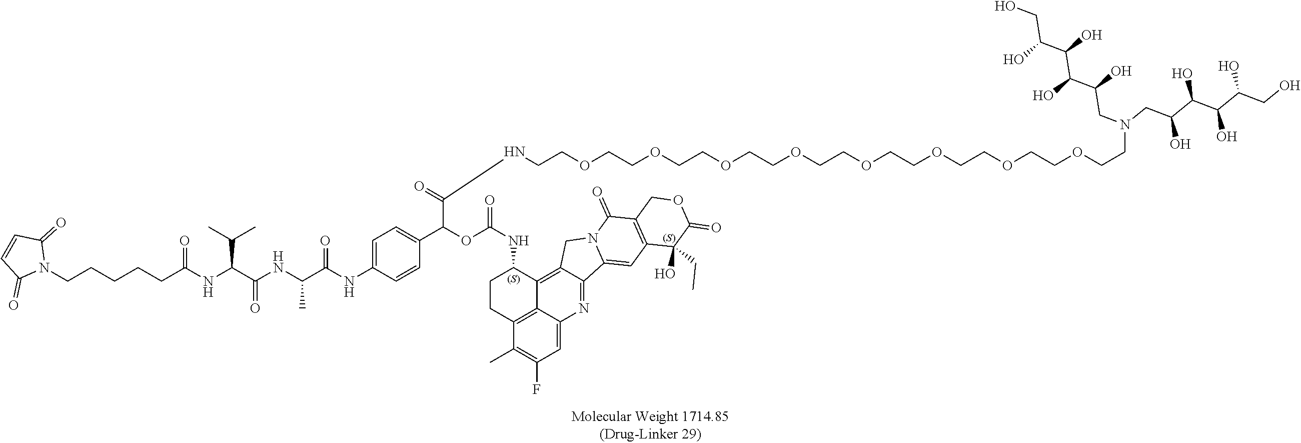

### Step 1

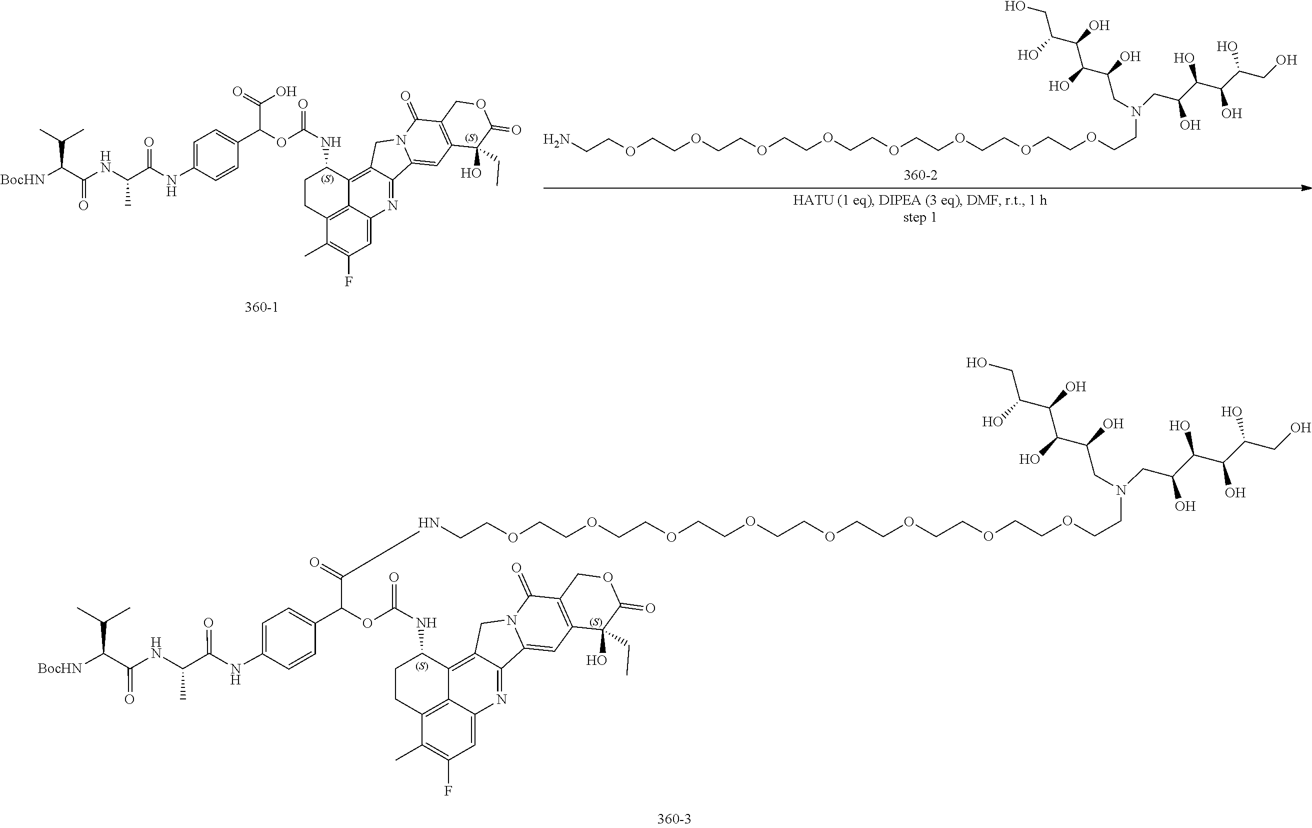

A solution of 360-1 (300 mg, 0.3 mmol), 360-2 (247 mg, 0.3 mmol) and HATU (127 mg, 0.3 mmol) in anhydrous DMF (6 mL) was stirred at room temperature for 5 min, then DIPEA (129 mg, 1.0 mmol) was added. The resulting solution was stirred for another 1 hr at r.t. until LCMS indicated complete reaction. The reaction solution was purified directly by reverse phase liquid chromatography to give 360-3 (130 mg, 0.08 mmol) as a gray solid. purity=90%-95%.

### Step 2

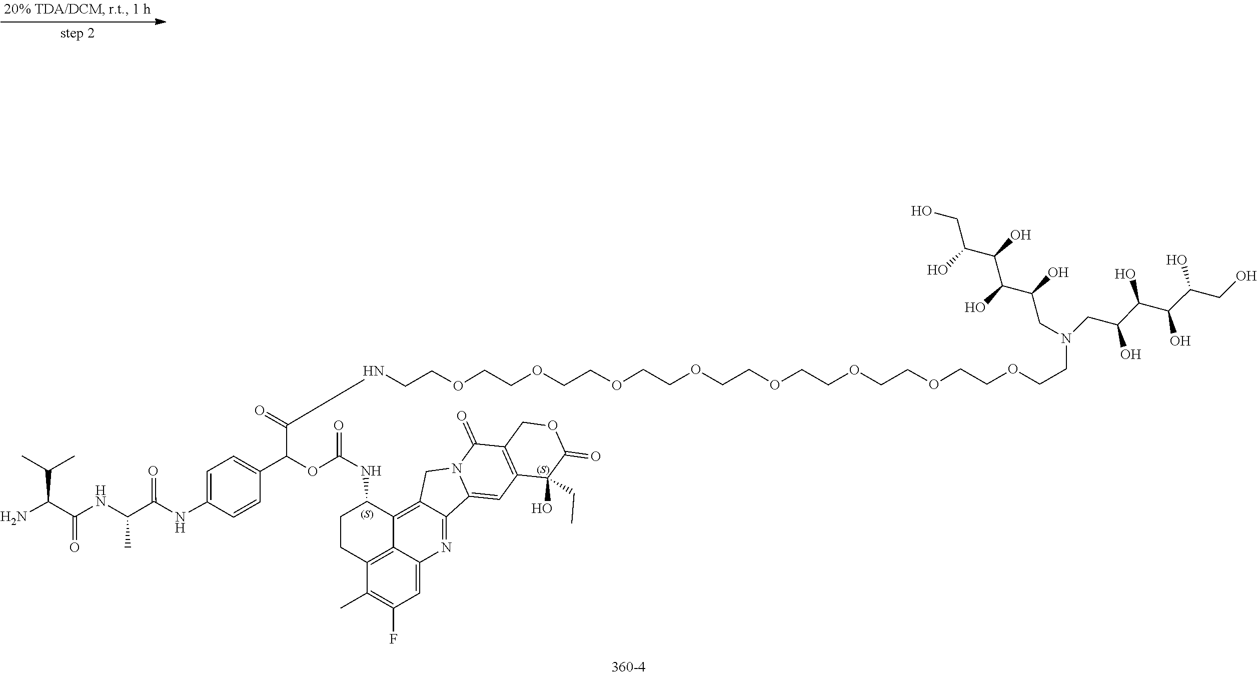

A solution of 360-3 (130 mg, 0.08 mmol) and TFA (1 mL) in anhydrous DCM (4 mL) was stirred at room temperature for 1 hr until LCMS indicated all starting amine was disappeared and desired product was detected. Then the solution was concentrated and purified by reverse phase liquid chromatography to give 360-4 (70 mg, 0.046 mmol) as a gray solid. purity=85%-90%.

### Step 3

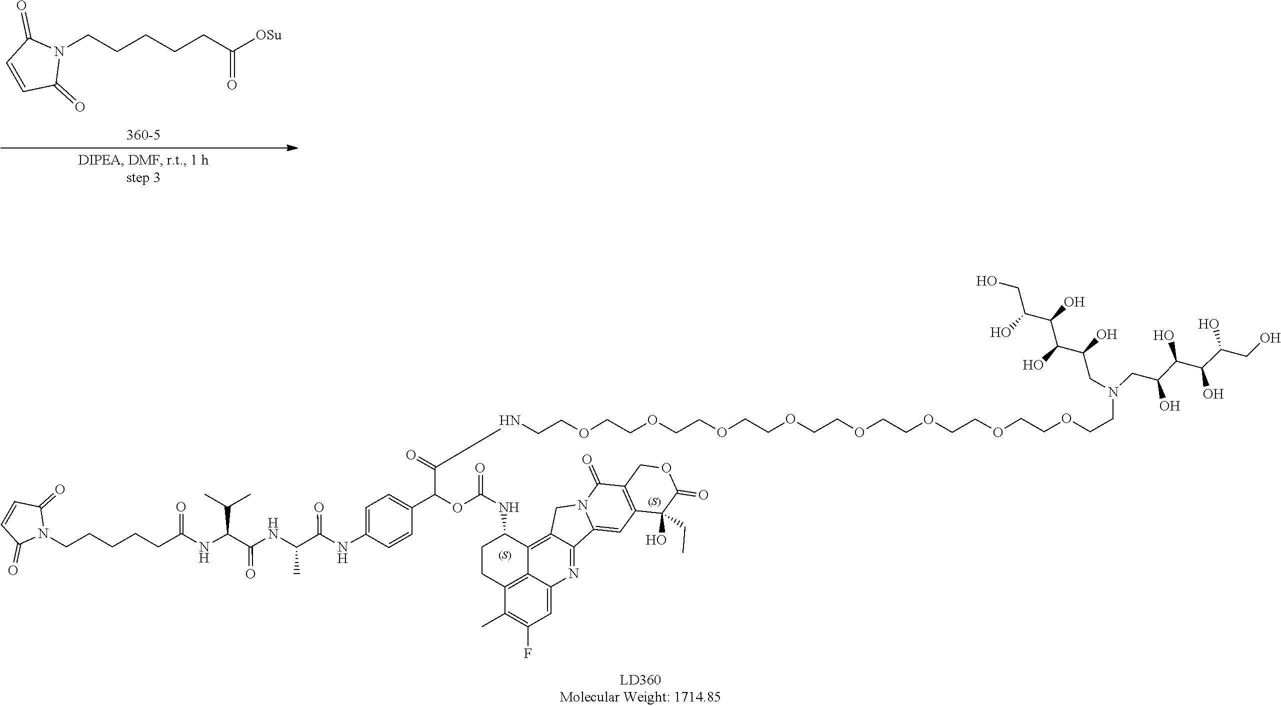

A solution of 360-4 (30 mg, 0.02 mmol) and DIPEA (8 mg, 0.06 mmol) in anhydrous DMF (1 mL) was stirred at room temperature for 5 min, then a solution of 360-5 (9 mg, 0.03 mmol) in anhydrous DMF (1 mL) was added dropwise by syringe over 5 min. The resulting solution was stirred for another 1 hr at r.t. until LCMS indicated complete reaction. Then the reaction solution was purified by prep. HPLC to give LD360 (3 mg) as yellow solid. LCMS, m/z=867.42 (M/2+H)⁺.

### Example 30: Preparation of Drug-Linker 30

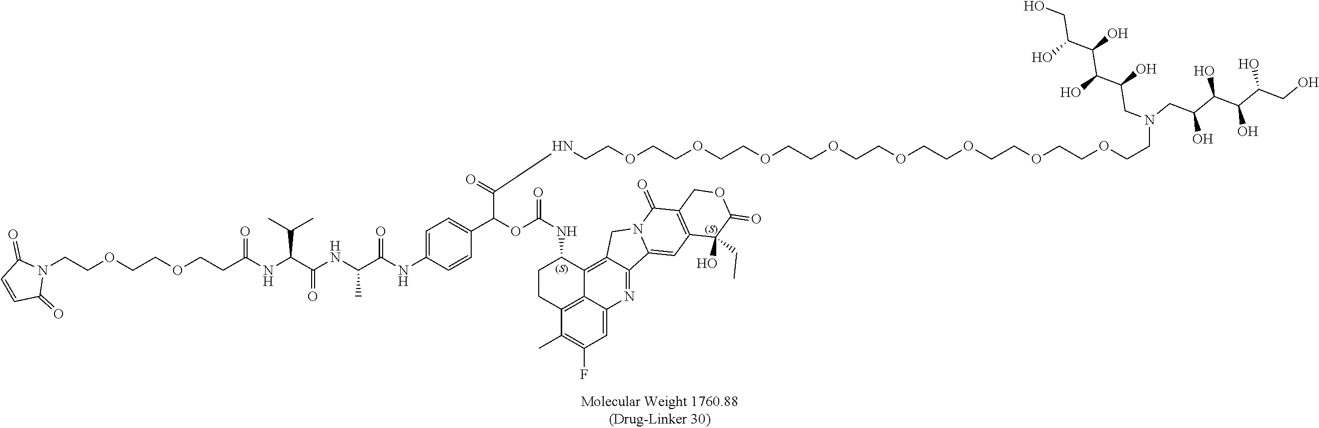

### Step 1

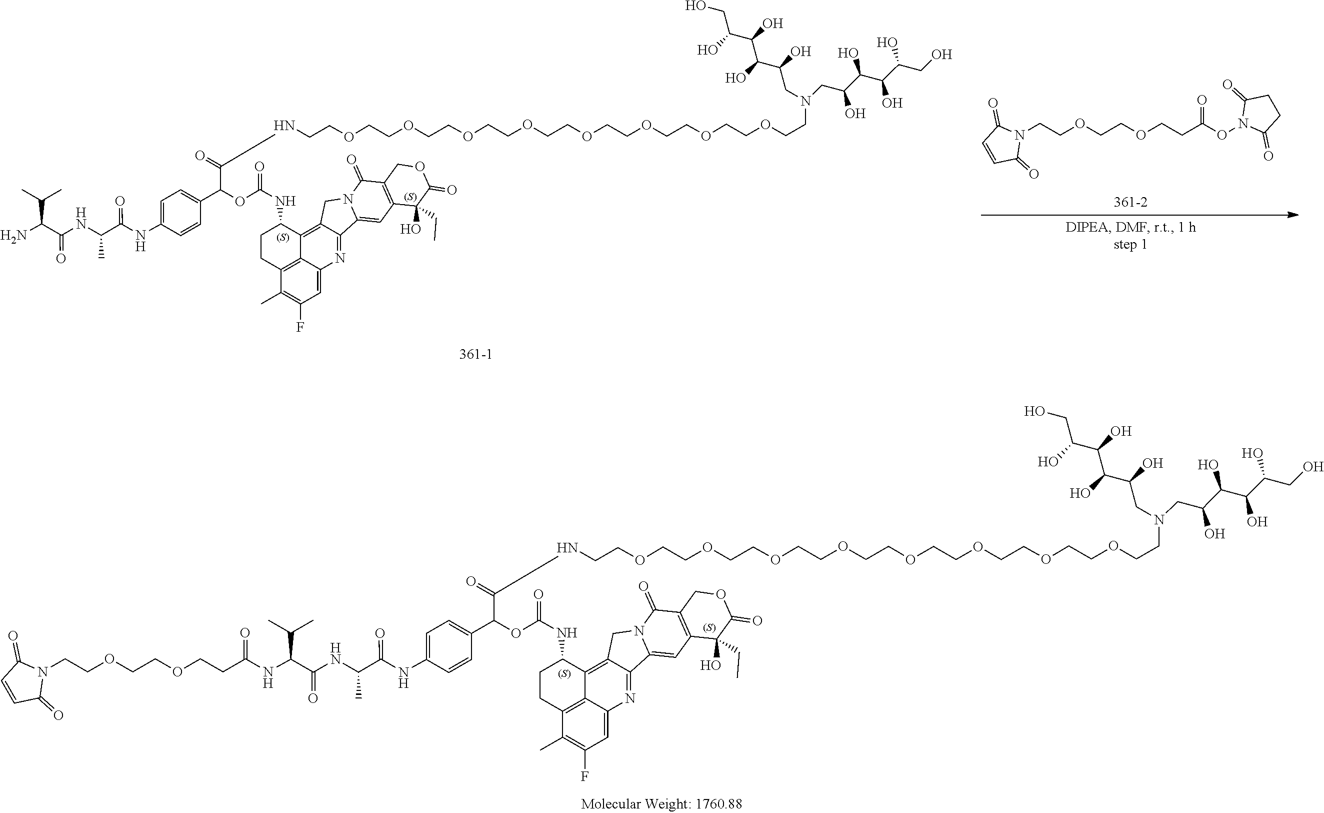

A solution of 361-1 (40 mg, 0.026 mmol) and DIPEA (8 mg, 0.062 mmol) in anhydrous DMF (1 mL) was stirred at room temperature for 5 min, then a solution of 360-5 (14 mg, 0.039 mmol) in anhydrous DMF (1 mL) was added dropwise by syringe over 5 min. The resulting solution was stirred for another 1 hr at r.t. until LCMS indicated complete reaction. Then the reaction solution was purified by prep. HPLC to give LD361 (11 mg) as yellow solid. LCMS, m/z=881.43 (M/2+H)⁺.

### Example 31: Preparation of Drug-Linker 31

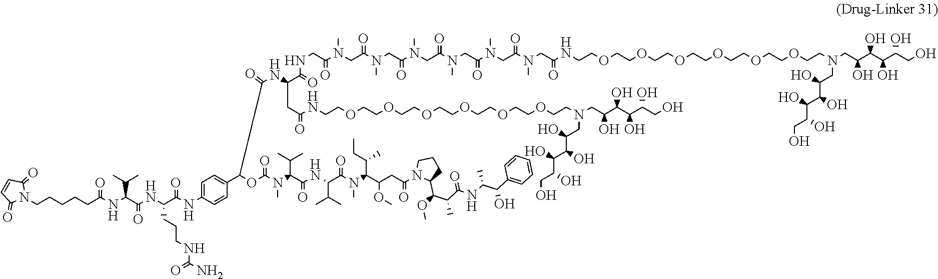

### Step 1

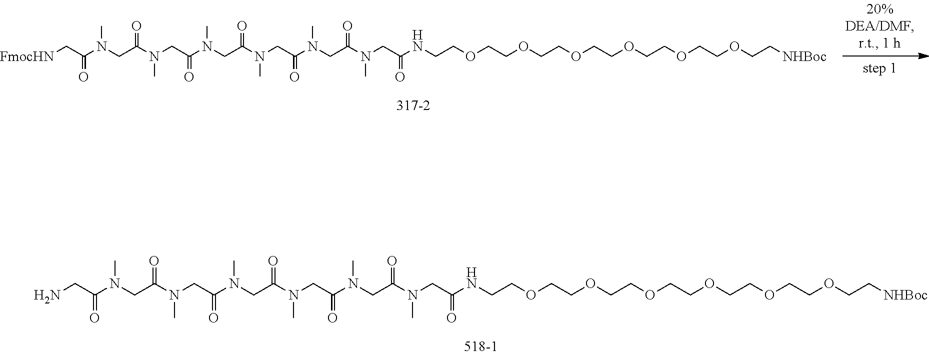

A solution of 317-2 (570 mg, 0.504 mmol) and DEA (1 mL) in anhydrous DMF (4 mL) was stirred at room temperature for 1 hr until LCMS indicated all starting amine was disappeared and desired product was detected. Then the solution was concentrated to dryness to 518-1 (419 mg, 0.461 mmol, 91.47%) as colorless oil, used as such in the next step. purity=90%-95%.

### Step 2

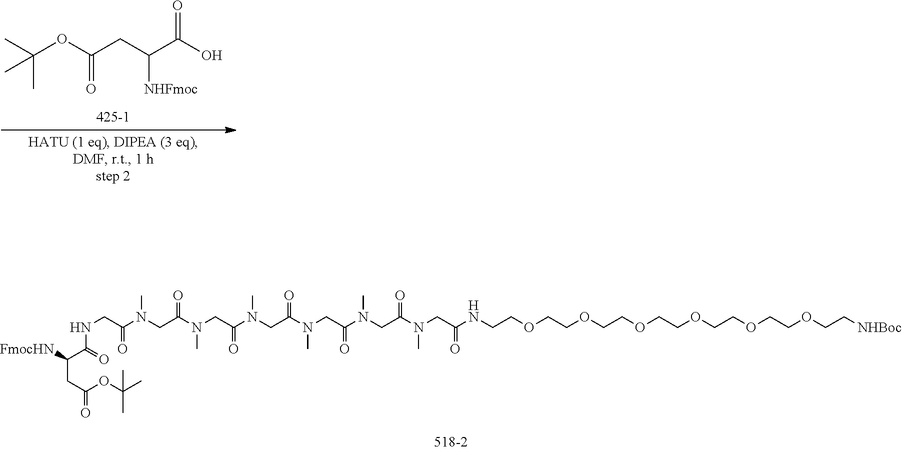

A solution of 518-1 (419 mg, 0.461 mmol), 425-1 (190 mg, 0.462 mmol) and HATU (175 mg, 0.460 mmol) in anhydrous DMF (10 mL) was stirred at room temperature for 5 min, then DIPEA (179 mg, 1.385 mmol) was added. The resulting solution was stirred for another 1 hr at r.t. until LCMS indicated complete reaction. The reaction solution was purified directly by reverse phase liquid chromatography to give 518-2 (305 mg, 0.234 mmol, 50.83%) as a white solid. purity=90%-95%.

### Step 3

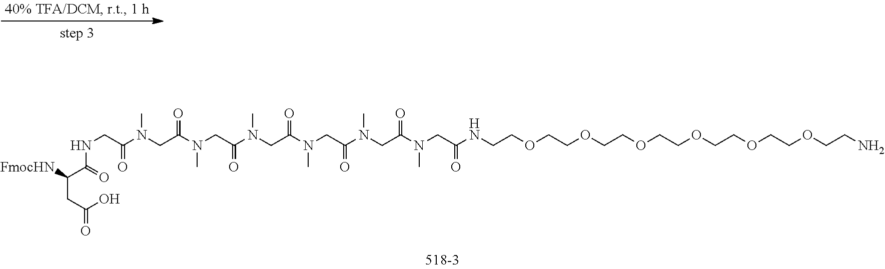

A solution of 518-2 (305 mg, 0.234 mmol) and TFA (4 mL) in anhydrous DCM (6 mL) was stirred at room temperature for 1 hr until LCMS indicated all starting amine was disappeared and desired product was detected. Then the solution was concentrated to dryness to 518-3 (255 mg, 0.223 mmol, 95.30%) as yellow oil, used as such in the next step. purity=90%-95%.

### Step 4

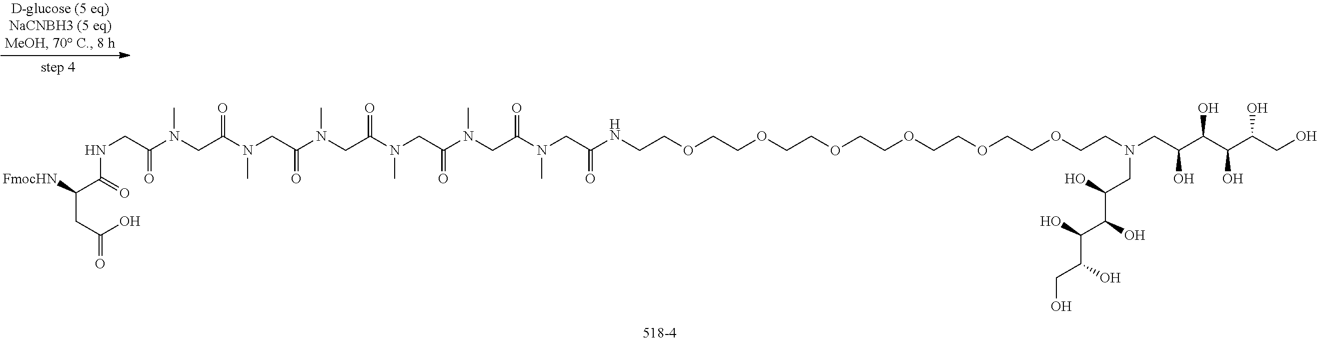

A solution of 518-3 (255 mg, 0.223 mmol) and D-Glucose (201 mg, 1.116 mmol) in anhydrous Methanol (40 mL) was heated at 50° C. for 30 min, then NaCNBH₃ (70 mg, 1.114 mmol) was added. The resulting solution was stirred for another 8 hr at 70° C. until LCMS indicated all starting amine was disappeared and the mass of desired product was detected. Then the reaction solution was concentrated and purified by reverse phase liquid chromatography to give 518-4 (180 mg, 0.122 mmol, 54.71%) as a white solid. purity=90%-95%.

### Step 5

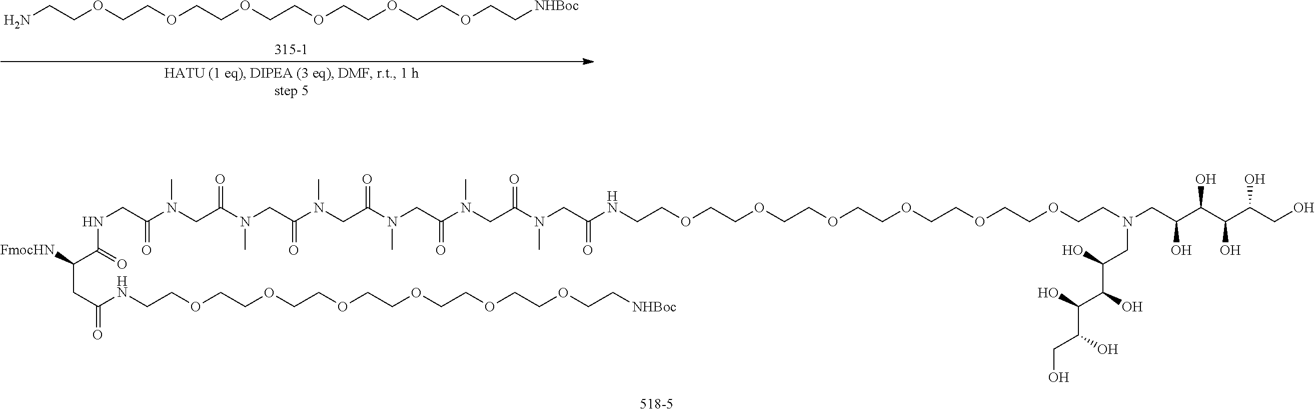

A solution of 518-4 (180 mg, 0.122 mmol), 315-1 (52 mg, 0.122 mmol) and HATU (46 mg, 0.121 mmol) in anhydrous DMF (5 mL) was stirred at room temperature for 5 min, then DIPEA (47 mg, 0.364 mmol) was added. The resulting solution was stirred for another 1 hr at r.t. until LCMS indicated complete reaction. The reaction solution was purified directly by reverse phase liquid chromatography to give 518-5 (166 mg, 0.088 mmol, 72.13%) as a white solid. purity=90%-95%.

### Step 6

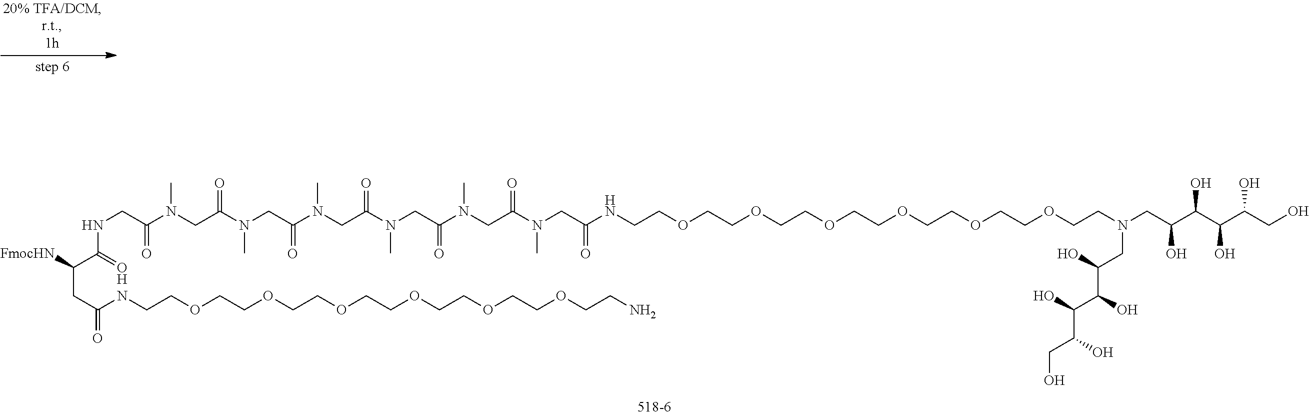

A solution of 518-5 (166 mg, 0.088 mmol) and TFA (1 mL) in anhydrous DCM (4 mL) was stirred at room temperature for 1 hr until LCMS indicated all starting amine was disappeared and desired product was detected. Then the solution was concentrated to dryness to 518-6 (147 mg, 0.083 mmol, 94.32%) as yellow oil, used as such in the next step. purity=90%-95%.

### Step 7

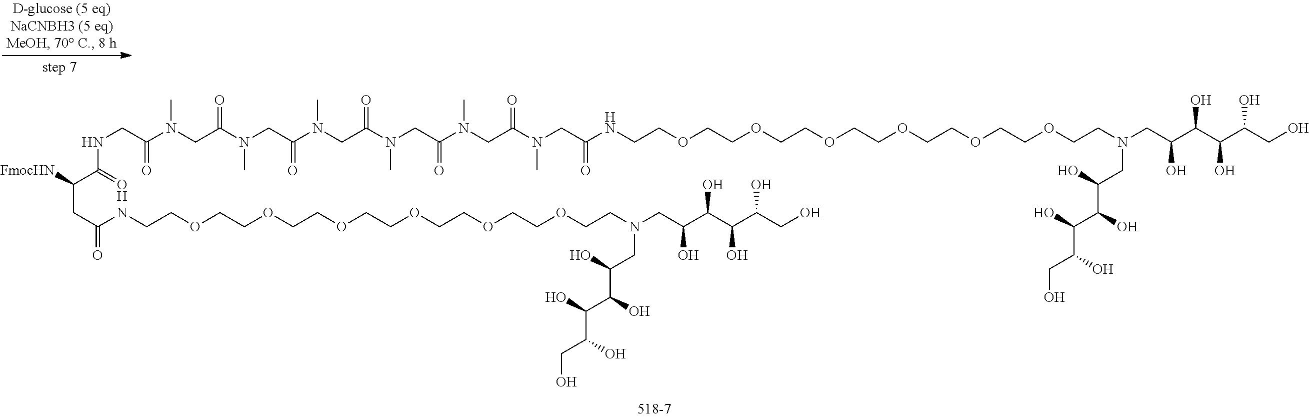

A solution of 518-6 (147 mg, 0.083 mmol) and D-Glucose (75 mg, 0.416 mmol) in anhydrous Methanol (40 mL) was heated at 50° C. for 30 min, then NaCNBH₃ (26 mg, 0.413 mmol) was added. The resulting solution was stirred for another 8 hr at 70° C. until LCMS indicated all starting amine was disappeared and the mass of desired product was detected. Then the reaction solution was concentrated and purified by reverse phase liquid chromatography to give 518-7 (95 mg, 0.045 mmol, 54.22%) as a white solid. purity=90%-95%.

### Step 8

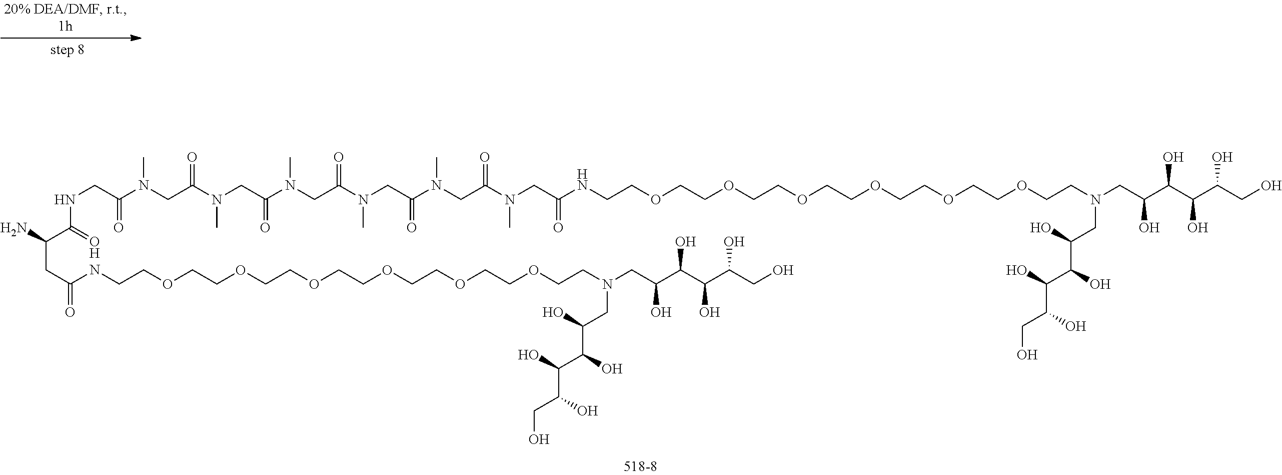 [SMILES: CN(CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)CN(C)C(=O)CN(C)C(=O)CN(C)C(=O)CN(C)C(=O)CN(C)C(=O)CNC(=O)[C@H](N)CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO]

A solution of 518-7 (95 mg, 0.045 mmol) and DEA (1 mL) in anhydrous DMF (4 mL) was stirred at room temperature for 1 hr until LCMS indicated all starting amine was disappeared and desired product was detected. Then the solution was concentrated to dryness to 518-8 (78 mg, 0.041 mmol, 91.11%) as colorless oil, used as such in the next step. purity=90%-95%.

### Step 9

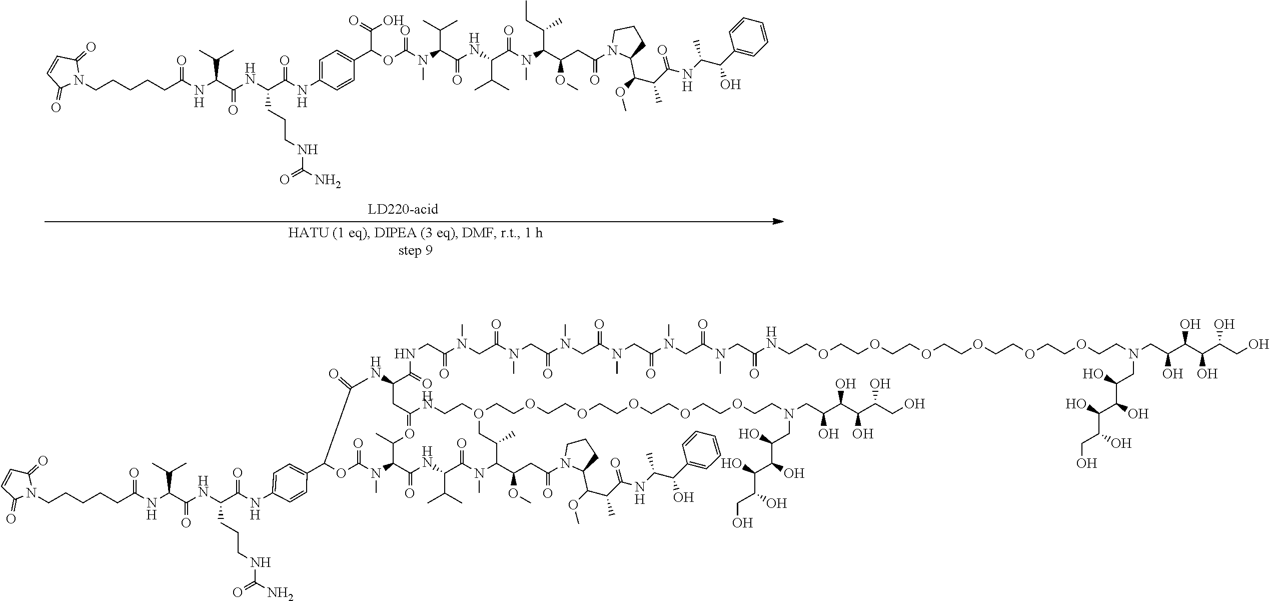

A solution of 518-8 (78 mg, 0.041 mmol), LD220-acid (56 mg, 0.041 mmol) and HATU (16 mg, 0.042 mmol) in anhydrous DMF (4 mL) was stirred at room temperature for 5 min, then DIPEA (16 mg, 0.124 mmol) was added. The resulting solution was stirred for another 1 hr at r.t. until LCMS indicated complete reaction. Then the reaction solution was purified by prep. HPLC to give LD518 (30 mg, 0.009 mmol, 21.95%) as a white solid. LCMS, m/z=1614.53 (M/2+H)⁺.

### Example 32: Preparation of Drug-Linker 32

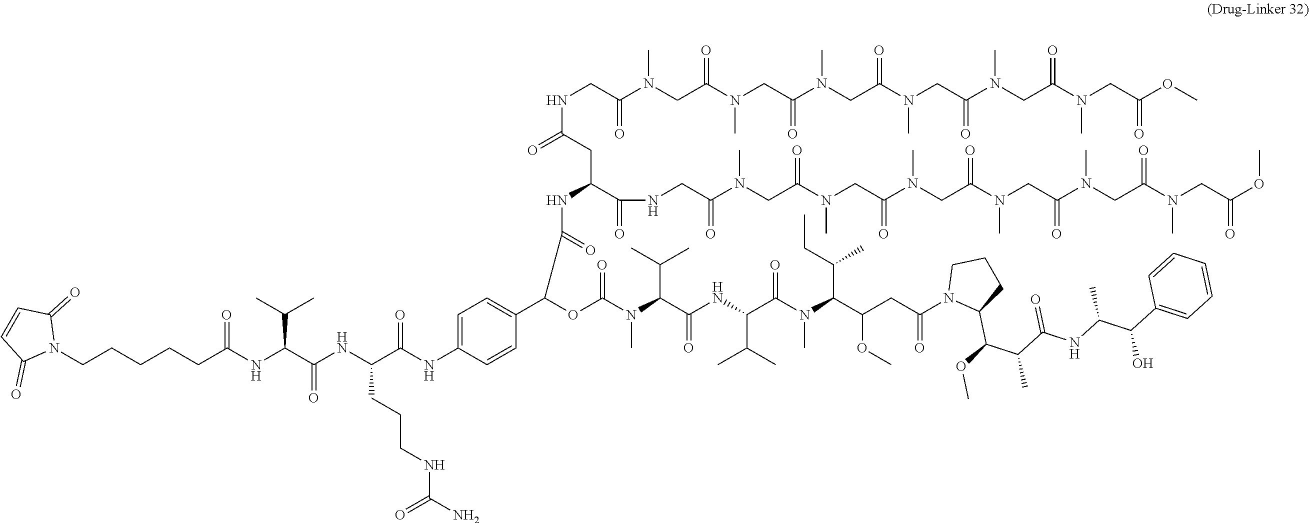 [SMILES: CC[C@H](C)[C@@H](C(CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@@H](CC(=O)NCC(=O)N(C)CC(=O)N(C)CC(=O)N(C)CC(=O)N(C)CC(=O)N(C)CC(=O)N(C)CC(=O)OC)C(=O)NCC(=O)N(C)CC(=O)N(C)CC(=O)N(C)CC(=O)N(C)CC(=O)N(C)CC(=O)N(C)CC(=O)OC)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

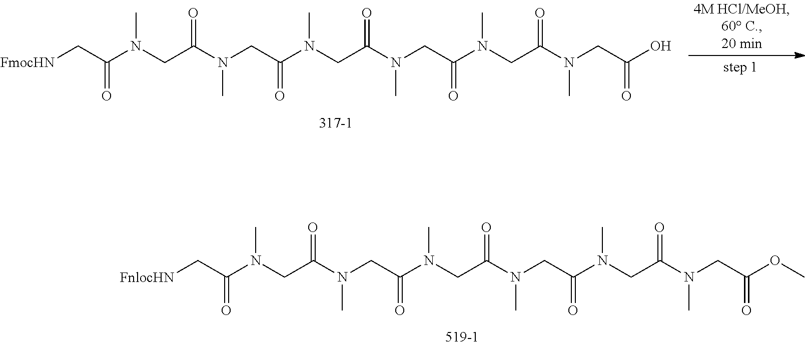

A solution of 317-1 (500 mg, 0.691 mmol) in 4M HCl/MeOH (10 ml) was heated at 50° C. for 30 min until LCMS indicated all starting amine was disappeared and the mass of desired product was detected. Then the reaction solution was concentrated and purified by reverse phase liquid chromatography to give 519-1 (367 mg, 0.507 mmol, 73.37%) as a white solid. purity=90%-95%.

### Step 2

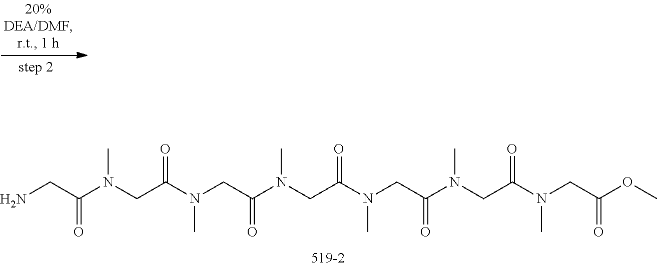 [SMILES: COC(=O)CN(C)C(=O)CN(C)C(=O)CN(C)C(=O)CN(C)C(=O)CN(C)C(=O)CN(C)C(=O)CN]

A solution of 519-1 (367 mg, 0.507 mmol) and DEA (1 mL) in anhydrous DMF (4 mL) was stirred at room temperature for 1 hr until LCMS indicated all starting amine was disappeared and desired product was detected. Then the solution was concentrated to dryness to 519-2 (233 mg, 0.452 mmol, 89.15%) as colorless oil, used as such in the next step. purity=90%-95%.

### Step 3

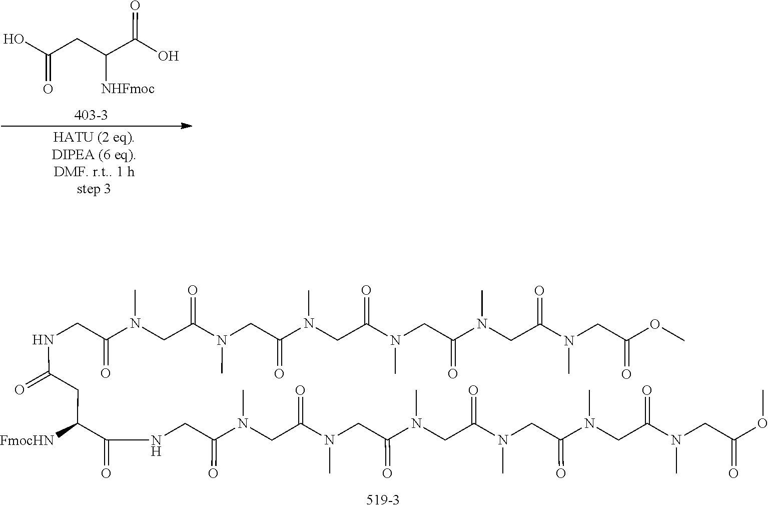

A solution of 519-2 (233 mg, 0.452 mmol), 403-3 (80 mg, 0.225 mmol) and HATU (172 mg, 0.452 mmol) in anhydrous DMF (5 mL) was stirred at room temperature for 5 min, then DIPEA (175 mg, 1.354 mmol) was added. The resulting solution was stirred for another 1 hr at r.t. until LCMS indicated complete reaction. The reaction solution was purified directly by reverse phase liquid chromatography to give 519-3 (185 mg, 0.137 mmol, 60.89%) as a white solid. purity=90% -95%.

### Step 4

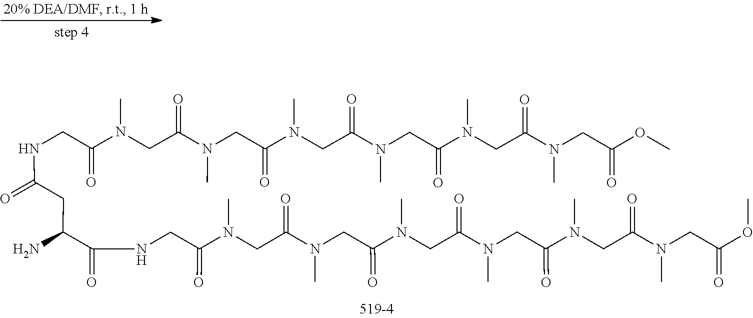 [SMILES: COC(=O)CN(C)C(=O)CN(C)C(=O)CN(C)C(=O)CN(C)C(=O)CN(C)C(=O)CN(C)C(=O)CNC(=O)C[C@H](N)C(=O)NCC(=O)N(C)CC(=O)N(C)CC(=O)N(C)CC(=O)N(C)CC(=O)N(C)CC(=O)N(C)CC(=O)OC]

A solution of 519-3 (185 mg, 0.137 mmol) and DEA (1 mL) in anhydrous DMF (4 mL) was stirred at room temperature for 1 hr until LCMS indicated all starting amine was disappeared and desired product was detected. The reaction solution was purified directly by reverse phase liquid chromatography to give 519-4 (120 mg, 0.106 mmol, 77.37%) as a white solid. purity=90%-95%.

### Step 5

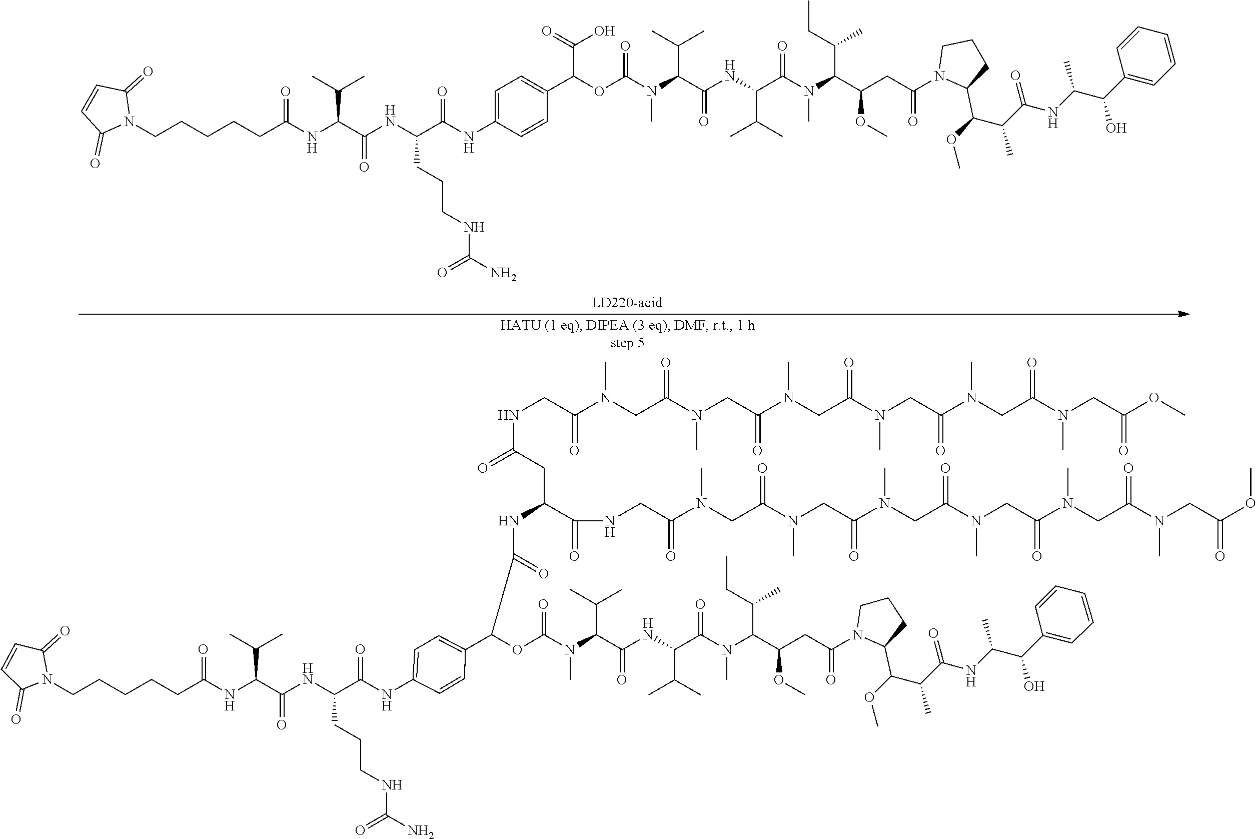

A solution of 519-4 (50 mg, 0.044 mmol), LD220-acid (60 mg, 0.044 mmol) and HATU (17 mg, 0.044 mmol) in anhydrous DMF (3 mL) was stirred at room temperature for 5 min, then DIPEA (17 mg, 0.132 mmol) was added. The resulting solution was stirred for another 1 hr at r.t. until LCMS indicated complete reaction. Then the reaction solution was purified by prep. HPLC to give LD519 (28 mg, 0.011 mmol, 25.75%) as a white solid. LCMS, m/z=1235.85 (M/2+H)⁺.

### Example 33: Preparation of Drug-Linker 33

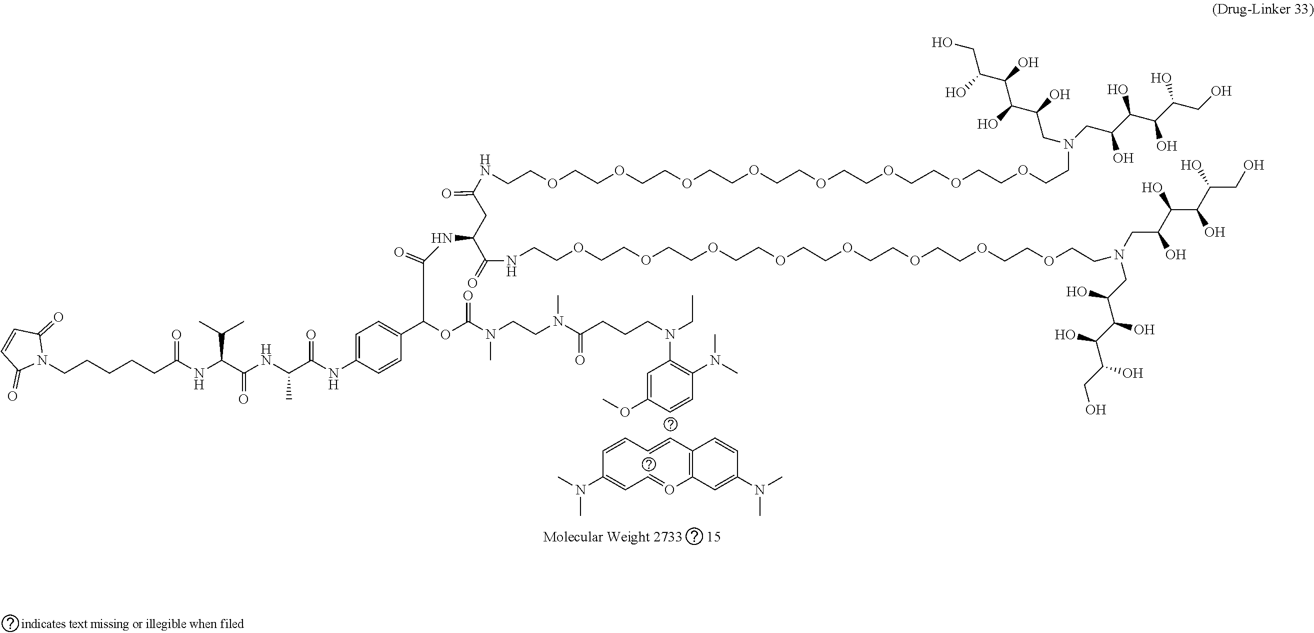

### Step 1

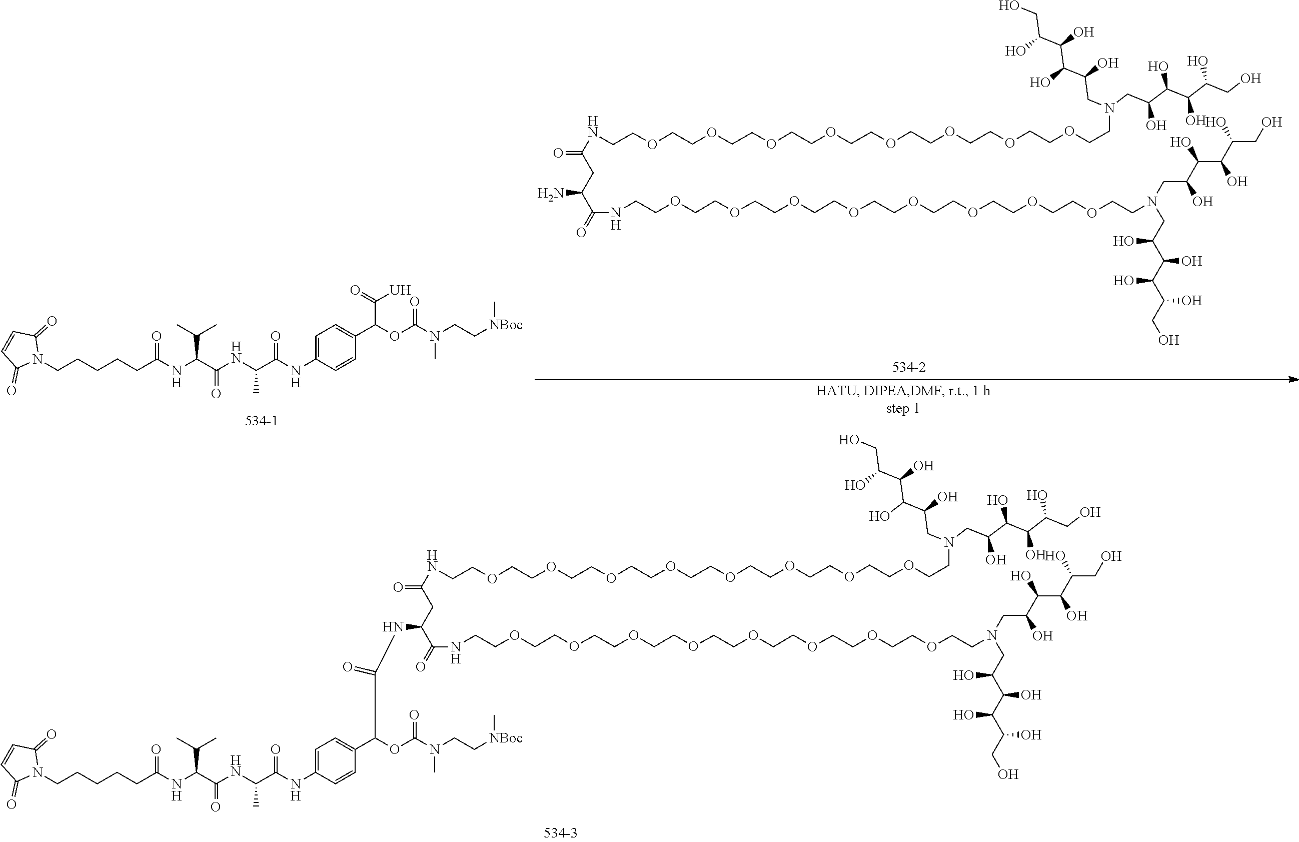

A solution of 534-1 (200 mg, 0.3 mmol), 534-2 (424 mg, 0.3 mmol) and HATU (102 mg, 0.3 mmol) in anhydrous DMF (4 mL) was stirred at room temperature for 5 min, then DIPEA (104 mg, 0.8 mmol) was added. The resulting solution was stirred for another 1 hr at r.t. until LCMS indicated complete reaction. The reaction solution was purified directly by reverse phase liquid chromatography to give 534-3 (140 mg, 0.06 mmol) as a white solid, purity=90%-95%.

### Step 2

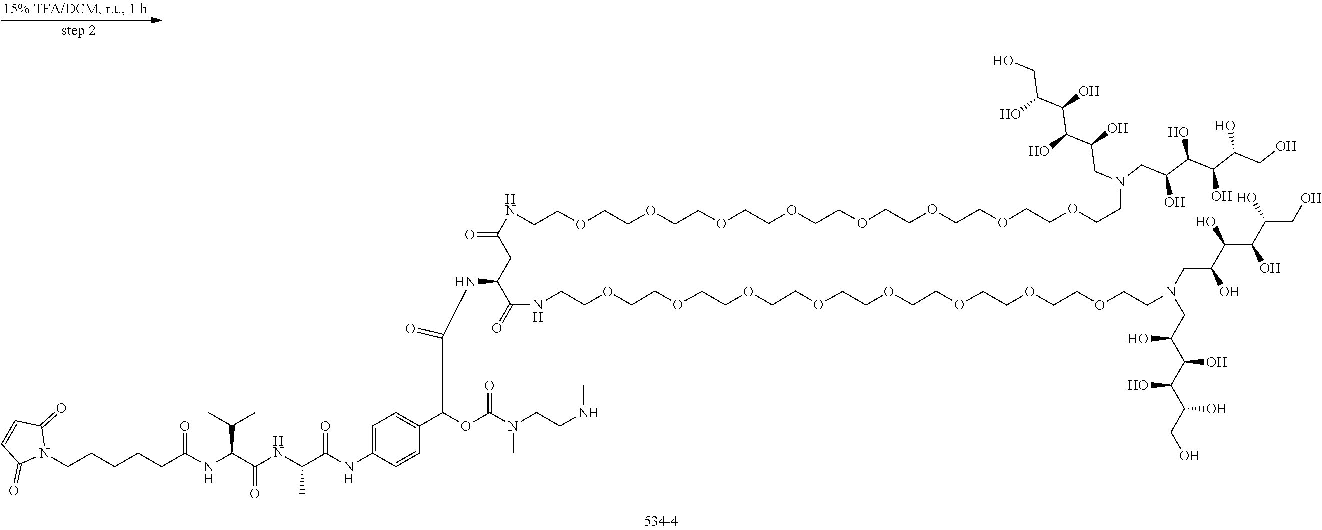 [SMILES: CNCCN(C)C(=O)OC(C(=O)N[C@@H](CC(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](C)NC(=O)[C@@H](NC(=O)CCCCCN2C(=O)C=CC2=O)C(C)C)cc1]

A solution of 534-3 (140 mg, 0.06 mmol) and TFA (1 mL) in anhydrous DCM (4 mL) was stirred at room temperature for 1 hr until LCMS indicated all starting amine was disappeared and desired product was detected. Then the solution was concentrated and purified by reverse phase liquid chromatography to give 534-4 (80 mg, 0.036 mmol) as a white solid, purity=85%-90%.

### Step 3

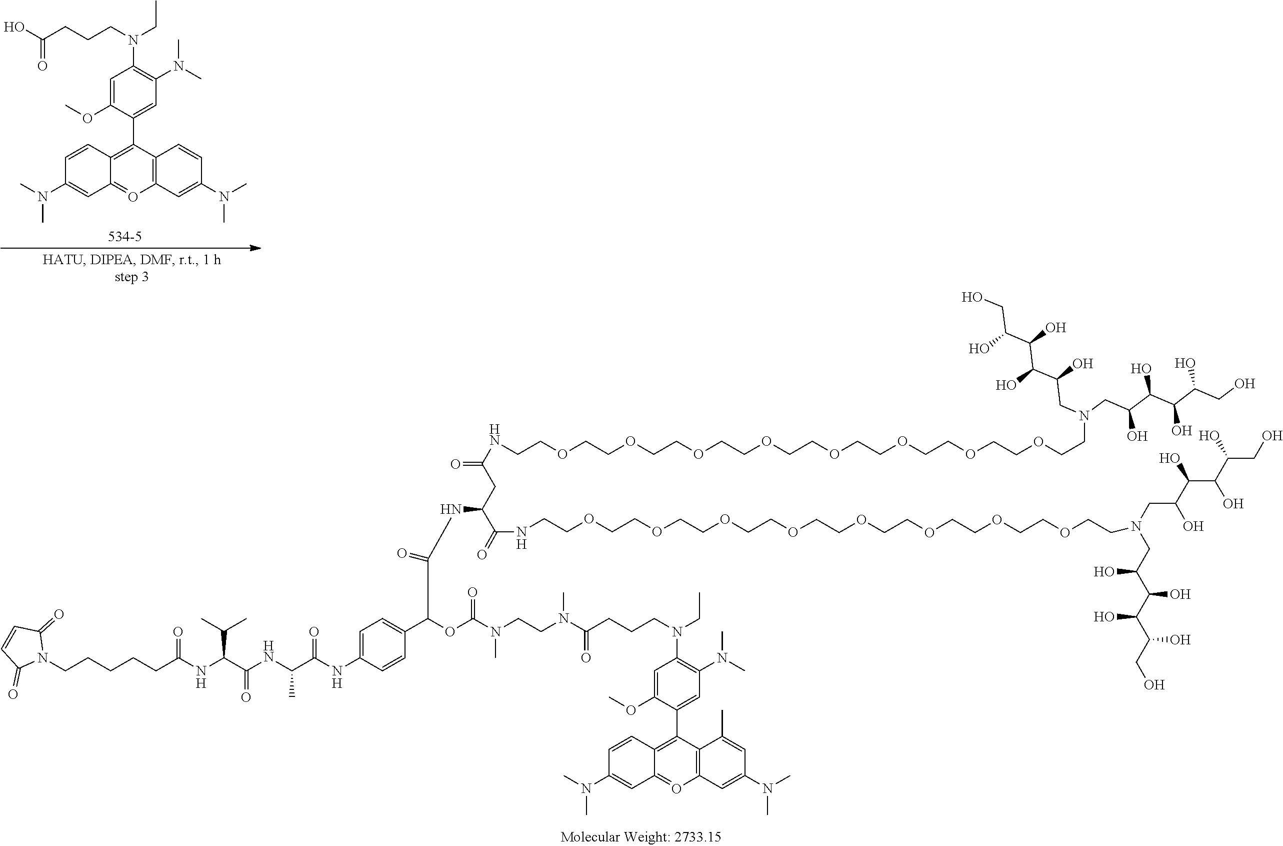

A solution of 534-4 (80 mg, 0.036 mmol), 534-5 (20 mg, 0.036 mmol) and HATU (14 mg, 0.036 mmol) in anhydrous DMF (2 mL) was stirred at room temperature for 5 min, then DIPEA (14 mg, 0.108 mmol) was added. The resulting solution was stirred for another 1 hr at r.t. until LCMS indicated complete reaction. Then the reaction solution was purified by prep. HPLC to give LD534 (13 mg, purity=80%) as purple solid. LCMS, m/z=1367.57 (M/2+H)⁺.

### Example 34: Preparation of Drug-Linker 34

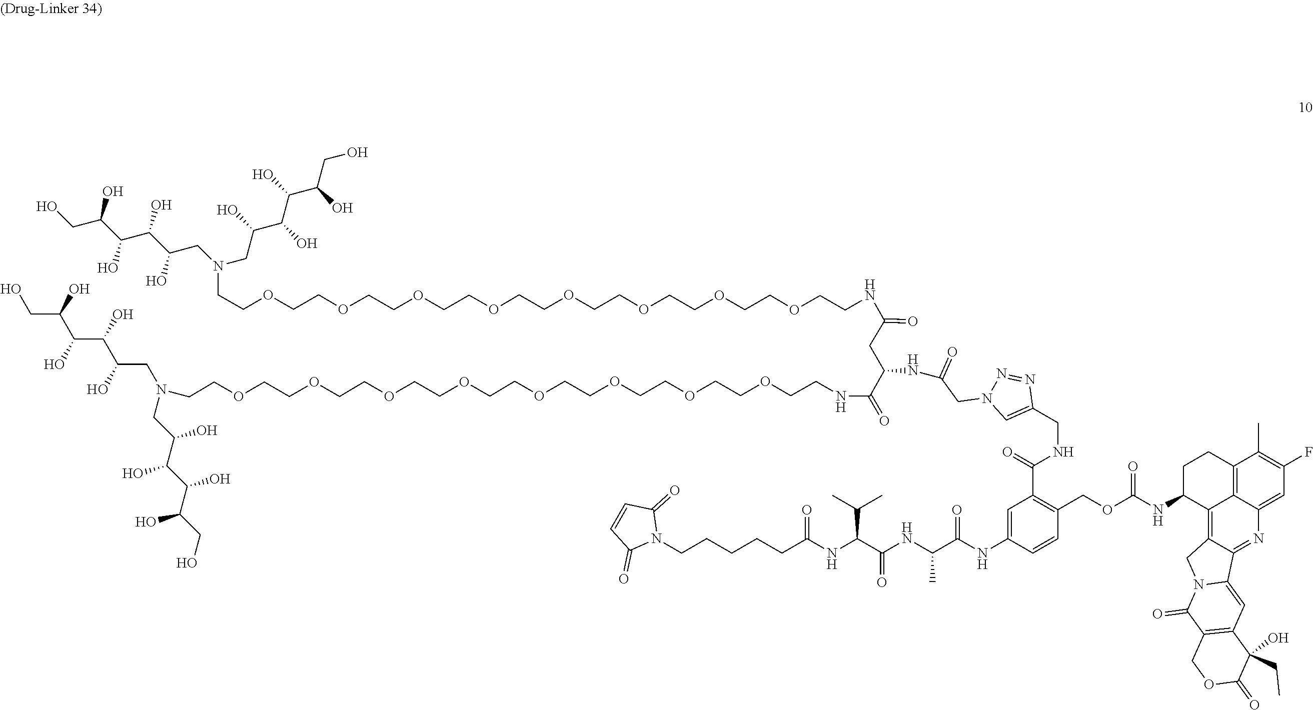 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)CCCCCN2C(=O)C=CC2=O)C(C)C)cc1C(=O)NCc1cn(CC(=O)N[C@@H](CC(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)nn1)CC3]

### Step 1

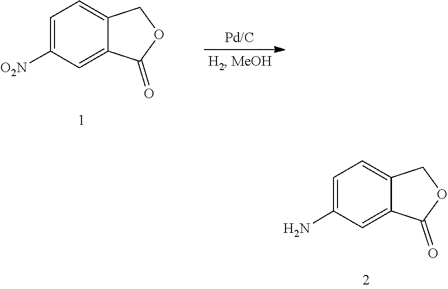 [SMILES: O=C1OCc2ccc([N+](=O)[O-])cc21 ; Nc1ccc2c(c1)C(=O)OC2]

A solution of 6-nitroisobenzofuran-1(3)-one (720 mg, 4 mmol) in MeOH (10 mL(was added 2003 mg Pd/C. The mixture was stirred at room temperature under H2 atmosphere until TLC indicated SM was consumed. Then the mixture was added 40 mL DCM and filtered with diatomaceous earth. The filtrate was concentrated to give 6-aminoisobenzofuran-1(3H)-one (600 mg, yield 98%) as a yellow solid. LCMS: m/z=150.3 (M+H)⁺, retention time 0.71 mine (0.01% TFA).

### Step 2

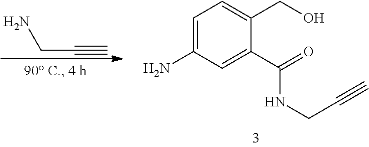

To a mixture of 6-aminoisobenzofuran-1(3H)-one (600 mg, 4 mmol) in a 100 mL flask was added 2 mL propargylamine. The mixture was stirred at 80° C. and stirred for 4 hrs. The mixture was concentrated under reduced pressure and purified by silica gel column to obtain 5-amino-2-(hydroxymethyl)-N-(prop-2-yn-1-yl)benzamide (570 mg, yield 70%) as a yellow solid. LCMS: m/z=227.0 (M+Na)⁺, retention time 1.03 min (0.01% TFA).

### Step 3

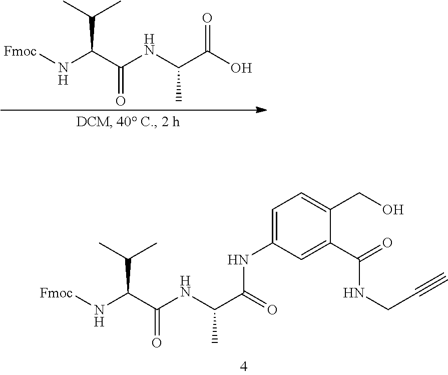

To a solution of 5-amino-2-(hydroxymethyl)-N-(prop-2-yn-1-yl)benzamide (570 mg, 2.8 mmol) in DCM (20 mL) was added (((9H-fluoren-9-yl)methoxy)carbonyl)-L-valyl-L-alanine (1.15 mg, 2.8 mmol) and stirred at 40° C. Then EEDQ (2.7 g, 11.2 mmol) was added, and the mixture was warmed to 45° C. for 2 h. Then the mixture was purified by silica gel column (DCM/MeOH=20/1, v/v) to give product (9H-fluoren-9-yl)methyl ((S)-1-(((S)-1-((4-(hydroxymethyl)-3-(prop-2-yn-1-ylcarbamoyl)phenyl)amino)-1-oxopropan-2-yl)amino)-3-methyl-1-oxobutan-2-yl)carbamate (1 g, yield 60%) as a yellow solid. LCMS: m/z=597.1 (M+H)⁺, retention time 5.58 min (0.01% TFA).

¹H NMR (400 MHz, DMSO-d₆) δ 10.11 (s, 1H), 8.85 (t, J=5.6 Hz, 1H), 8.22 (d, J=6.8 Hz, 1H), 7.89 (d, J=7.5 Hz, 2H), 7.75 (dd, J=7.5, 4.7 Hz, 2H), 7.70-7.62 (m, 2H), 7.48-7.39 (m, 4H), 7.34-7.30 (m, 2H), 5.25 (t, J=5.6 Hz, 1H), 4.51 (d, J=5.1 Hz, 2H), 4.42 (q, J=6.8 Hz, 1H), 4.35 (d, J=5.4 Hz, 1H), 4.31-4.26 (m, 1H), 4.25-4.18 (m, 3H), 4.00 (td, J=5.4, 2.5 Hz, 3H), 3.91 (dd, J=9.0, 7.1 Hz, 1H), 3.14 (dt, J=8.5, 2.5 Hz, 1H), 1.98 (q, J=6.8 Hz, 1H), 1.31 (d, J=7.1 Hz, 3H), 0.92-0.84 (m, 6H).

### Step 4

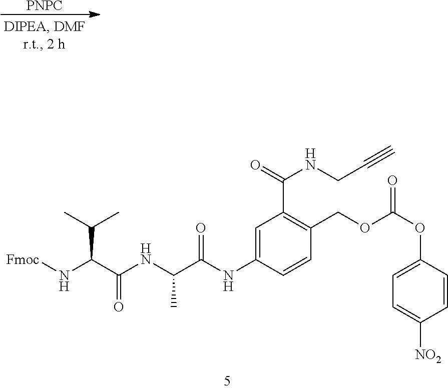

To a solution of (9H-fluoren-9-yl)methyl ((S)-1-(((S)-1-((4-(hydroxymethyl)-3-(prop-2-yn-1-ylcarbamoyl)phenyl)amino)-1-oxopropan-2-yl)amino)-3-methyl-1-oxobutan-2-yl)carbamate (340 mg, 0.57 mmol) and bis(4-nitrophenyl) carbonate (520 mg, 1.71 mmol) in DMF (6 mL) was added DIPEA (221 mg, 1.71 mmol). The reaction was stirred at room temperature for 1 h. The mixture was purified by reverse phase column chromatography (C18 column, eluting with 90% acetonitrile in water with 0.01% TFA) to get desired fractions, which was freeze-dried to get (9H-fluoren-9-yl)methyl ((S)-3-methyl-1-(((S)-1-((4-((((4-nitrophenoxy)carbonyl)oxy)methyl)-3-(prop-2-yn-1-ylcarbamoyl)phenyl)amino)-1-oxopropan-2-yl)amino)-1-oxobutan-2-yl)carbamate (150 mg, yield 34%) as a yellow solid. LCMS: m/z=762.2 (M+H)⁺, retention time 6.58 min (0.01% TFA).

### Step 5

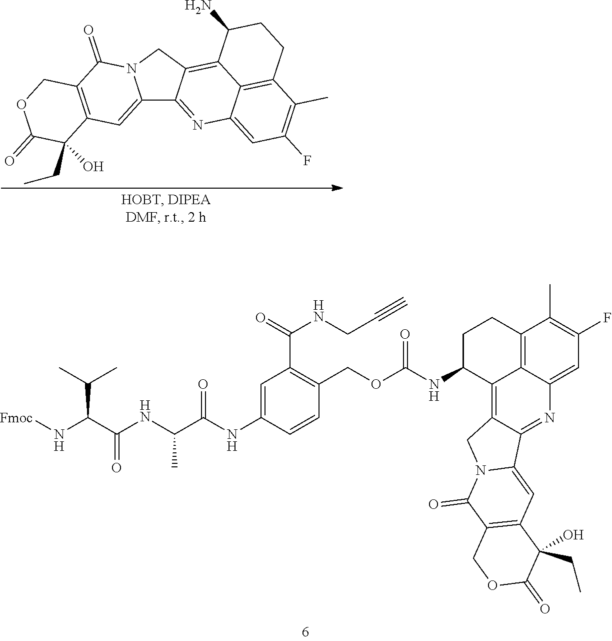

To a solution of (9H-fluoren-9-yl)methyl ((S)-3-methyl-1-(((S)-1-((4-((((4-nitrophenoxy)carbonyl)oxy)methyl)-3-(prop-2-yn-1-ylcarbamoyl)phenyl)amino)-1-oxopropan-2-yl)amino)-1-oxobutan-2-yl)carbamate (150 mg, 0.19 mmol) and Exatecan (127.6 mg, 0.24 mmol) in DMF (5 mL) was added DIPEA (98.2 mg, 0.76 mmol) and stirred at room temperature. Then HOBT (32.4, 0.24 mmol) was added, and the mixture was warmed to 25° C. for 2 hours to complete. Then the mixture concentrated and purified by silica gel column (DCM/MeOH=20/1, v/v) to give (9H-fluoren-9-yl)methyl ((S)-1-(((S)-1-((4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-3-(prop-2-yn-1-ylcarbamoyl)phenyl)amino)-1-oxopropan-2-yl)amino)-3-methyl-1-oxobutan-2-yl)carbamate (150 mg, yield 75%). LCMS: m/z=1058.6 (M+H)⁺, retention time 6.30 min (0.01% TFA).

¹H NMR (400 MHz, DMSO-d₆) δ 10.17 (s, 1H), 8.85 (t, J=5.5 Hz, 1H), 8.22 (d, J=6.8 Hz, 1H), 8.07 (d, J=8.8 Hz, 1H), 7.88 (d, J=7.5 Hz, 2H), 7.78 (d, J=10.9 Hz, 1H), 7.75-7.68 (m, 3H), 7.50-7.37 (m, 4H), 7.35-7.25 (m, 3H), 6.54 (s, 1H), 5.44 (s, 2H), 5.35-5.14 (m, 5H), 4.40 (t, J=7.0 Hz, 1H), 4.32-4.26 (m, 1H), 4.21 (d, J=5.1 Hz, 2H), 4.01-3.95 (m, 2H), 3.91 (t, J=8.1 Hz, 1H), 3.23 (s, 1H), 3.16-3.06 (m, 2H), 2.41-2.34 (m, 3H), 2.19 (s, 2H), 1.99 (dt, J=13.6, 7.3 Hz, 2H), 1.86 (dt, J=17.6, 7.1 Hz, 2H), 1.30 (d, J=7.0 Hz, 3H), 0.87 (ddd, J=9.8, 6.8, 3.6 Hz, 9H).

¹³C NMR (101 MHz, DMSO-d₆) δ 171.68, 171.52, 168.55, 156.62, 144.34, 144.27, 141.17, 138.03, 135.46, 128.75, 128.13, 127.54, 125.87, 120.59, 118.48, 115.46, 81.51, 73.43, 66.14, 61.71, 60.97, 49.51, 47.13, 28.79, 19.69, 18.77, 18.53.

### Step 6

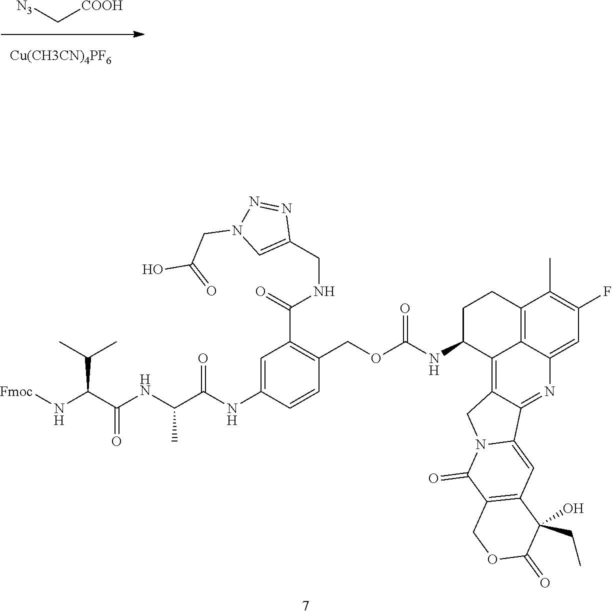

To a solution of (9H-fluoren-9-yl)methyl ((S)-1-(((S)-1-((4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-3-(prop-2-yn-1-ylcarbamoyl)phenyl)amino)-1-oxopropan-2-yl)amino)-3-methyl-1-oxobutan-2-yl)carbamate (53 mg, 0.05 mmol) and azidoacetic acid (7.6 mg, 0.075 mmol) in DMF (4 mL) was added Cu(CH₃CN)₄PF₆(28 mg, 0.075 mmol). The mixture was stirred at room temperature for 1 hr. The solvent was removed under reduced and purified by reversed phase column chromatography (0.01% TFA) to give product 2-(4-((5-((S)-2-((S)-2-((((9H-fluoren-9-yl)methoxy)carbonyl)amino)-3-methylbutanamido)propanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzamido)methyl)-1H-1,2,3-triazol-1-yl)acetic acid (36 mg, yield 62%) as a white solid. LCMS: m/z=1159.7 (M+H)⁺, retention time 5.70 min (0.01% TFA), weak UV.

### Step 7

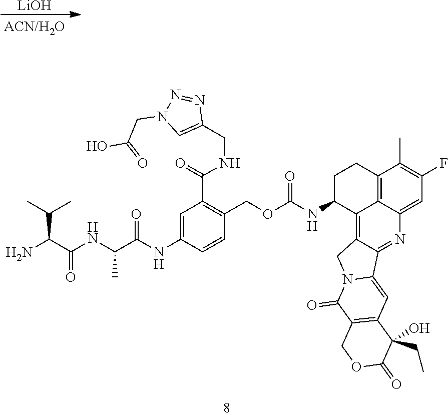 [SMILES: "LiOH" ; CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](N)C(C)C)cc1C(=O)NCc1cn(CC(=O)O)nn1)CC3]

To a solution 2-(4-((5-((S)-2-((S)-2-((((9H-fluoren-9-yl)methoxy)carbonyl)amino)-3-methylbutanamido)propanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzamido)methyl)-1H-1,2,3-triazol-1-yl)acetic acid (73 mg, 0.063 mmol) in THF (18 mL) and H₂O (6 mL) was added LiOH (10 mg, 0.252 mmol) at room temperature. Then the mixture was stirred at 25° C. for 1h until LCMS analysis showed major peak was product. The mixture was adjusted pH 7-8 by TFA, used directly in next step without further purification. LCMS: m/z=937.5 (M+H)⁺, retention time 3.37 min (0.01% TFA).

### Step 8

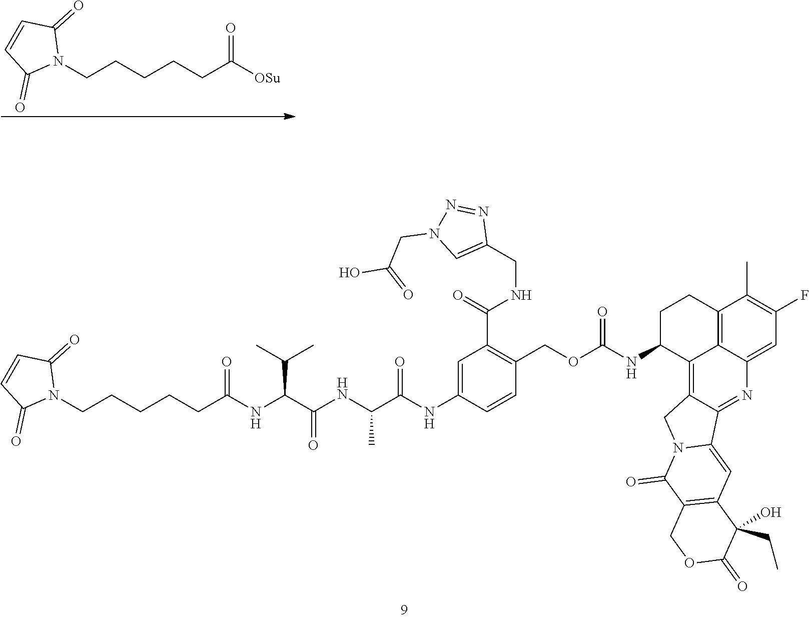

To the reaction in the previous step was added 2,5-dioxopyrrolidin-1-yl 6-(2,5-dioxo-2,5-dihydro-1H-pyrrol-1-yl)hexanoate (13.8 mg, 0.045 mmol) at room temperature and stirred for 2 h. The mixture was concentrated under reduced pressure and purified by reversed phase column chromatography (0.01% TFA) to give product 2-(4-((5-((S)-2-((S)-2-(6-(2,5-dioxo-2,5-dihydro-1H-pyrrol-1-yl)hexanamido)-3-methylbutanamido)propanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzamido)methyl)-1H-1,2,3-triazol-1-yl)acetic acid (18 mg, yield 40%) as a yellow solid. LCMS: m/z=1130.38 (M+H)⁺, retention time 4.32 min (0.01% TFA), weak UV.

### Step 9

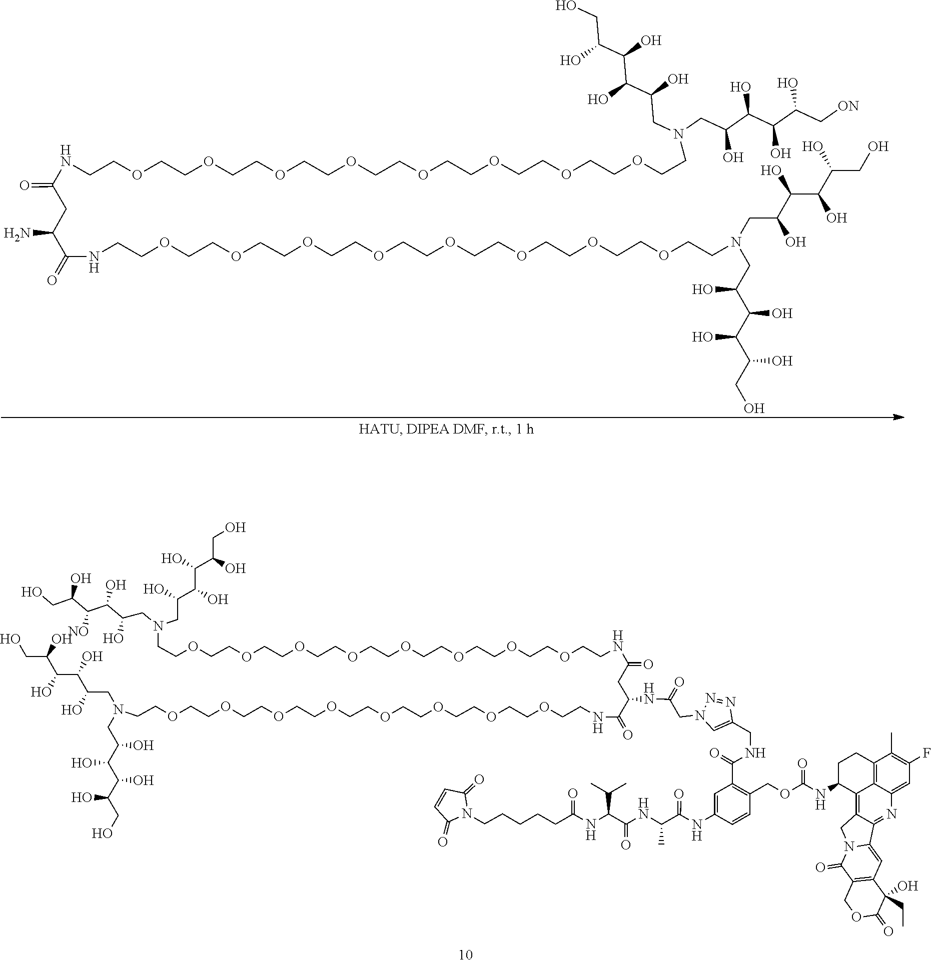

To a solution of 2-(4-((5-((S)-2-((S)-2-(6-(2,5-dioxo-2,5-dihydro-1H-pyrrol-1-yl)hexanamido)-3-methylbutanamido)propanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzamido)methyl)-1H-1,2,3-triazol-1-yl)acetic acid (18 mg, 0.015 mmol) and (S)-2-amino-N1,N4-bis((29S,30R,31R,32R)-29,30,31,32,33-pentahydroxy-27-((2S,3R,4R,5R)-2,3,4,5,6-pentahydroxyhexyl)-3,6,9,12,15,18,21,24-octaoxa-27-azatritriacontyl)succinamide (37 mg, 0.024 mmol) in DMF (5 mL) was added DIPEA (6.2 mg, 0.05 mmol) and stirred at room temperature. Then HATU (9.1 mg, 0.024 mmol) was added, and the mixture was warmed to 25° C. for 0.5h to complete. Then the mixture was purified by reversed phase column chromatography (0.01% TFA) to give product 4-((S)-2-((S)-2-(6-(2,5-dioxo-2,5-dihydro-1H-pyrrol-1-yl)hexanamido)-3-methylbutanamido)propanamido)-2-(((1-((4S,36S,37R,38R,39R)-36,37,38,39,40-pentahydroxy-2,6-dioxo-4-(((29S,30R,31R,32R)-29,30,31,32,33-pentahydroxy-27-((2S,3R,4R,5R)-2,3,4,5,6-pentahydroxyhexyl)-3,6,9,12,15,18,21,24-octaoxa-27-azatritriacontyl)carbamoyl)-34-((2S,3R,4R,5R)-2,3,4,5,6-pentahydroxyhexyl)-10,13,16,19,22,25,28,31-octaoxa-3,7,34-triazatetracontyl)-1H-1,2,3-triazol-4-yl)methyl)carbamoyl)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (22 mg, yield 55%) as a white solid. LCMS: m/z=1346.4 (M/2+H)⁺, retention time 3.60 min (0.01% TFA).

¹H NMR (400 MHz, DMSO-d₆) δ 10.09 (s, 1H), 8.93 (s, 1H), 8.55 (d, J=8.0 Hz, 1H), 8.22-8.13 (m, 2H), 8.07 (d, J=8.7 Hz, 1H), 8.01-7.89 (m, 3H), 7.79 (t, J=8.8 Hz, 1H), 7.73 (d, J=8.7 Hz, 1H), 7.65 (s, 1H), 7.46 (d, J=Hz, 1H), 7.31 (d, J=2.2 Hz, 1H), 7.00 (s, 1H), 6.53 (s, 1H), 5.44 (d, J=4.8 Hz, 4H), 5.32-5.18 (m, 4H), 5.14-5.05 (m, 2H), 4.80 (t, J=8.2 Hz, 4H), 4.66-4.49 (m, 8H), 4.45 (s, 5H), 4.36 (t, J=7.0 Hz, 1H), 4.16 (t J=7.8 Hz, 1H), 3.98 (s, 4H), 3.77 (s, 4H), 3.67 (d, J=6.5 Hz, 4H), 3.63-3.54 (m, 9H), 3.50 (d, J=2.6 Hz, 44H), 3.45-3.38 (m, 10H), 3.36 (d, J=6.2 Hz, 18H), 3.29-3.21 (m, 7H), 3.17 (s, 4H), 3.09 (dd, J=18.4, 7.8 Hz, 2H), 2.65 (s, 2H), 2.38 (d, J=2.0 Hz, 4H), 2.26-2.18 (m, 2H), 2.03-1.91 (m, 2H), 1.46 (t, J=7.6 Hz, 4H), 1.29 (d, J=7.0 Hz, 3H), 1.16 (t, J=7.3 Hz, 2H), 1.08 (t, J=7.1 Hz, 2H), 0.96-0.71 (m, 9H).

### Example 35: Preparation of Drug-Linker 35

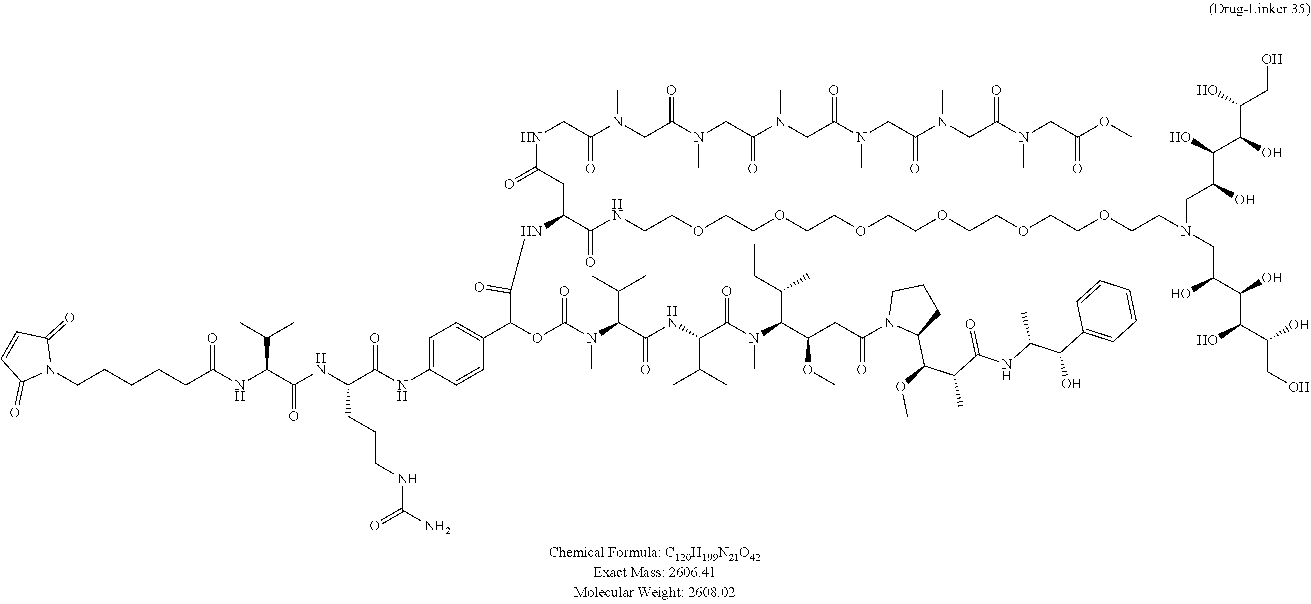 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@@H](CC(=O)NCC(=O)N(C)CC(=O)N(C)CC(=O)N(C)CC(=O)N(C)CC(=O)N(C)CC(=O)N(C)CC(=O)OC)C(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

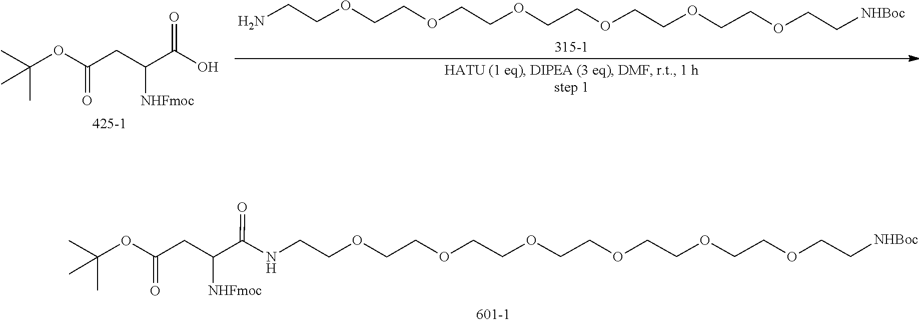

A solution of 425-1 (300 mg, 0.729 mmol), 315-1 (310 mg, 0.730 mmol) and HATU (277 mg, 0.728 mmol) in anhydrous DMF (10 mL) was stirred at room temperature for 5 min, then DIPEA (283 mg, 2.190 mmol) was added. The resulting solution was stirred for another 1 hr at r.t. until LCMS indicated complete reaction. The reaction solution was purified directly by reverse phase liquid chromatography to give 601-1 (375 mg, 0.458 mmol, 62.83%) as a white solid. purity=90%-95%.

### Step 2

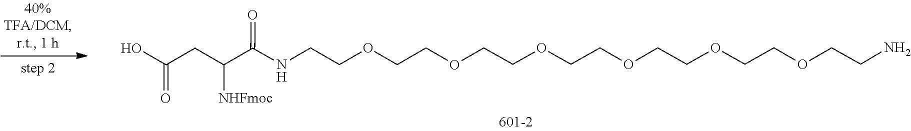

A solution of 601-1 (375 mg, 0.458 mmol) and TFA (4 mL) in anhydrous DCM (6 mL) was stirred at room temperature for 1 hr until LCMS indicated all starting amine was disappeared and desired product was detected. Then the solution was concentrated to dryness to 601-2 (271 mg, 0.410 mmol, 89.52%) as yellow oil, used as such in the next step. purity=90%-95%.

### Step 3

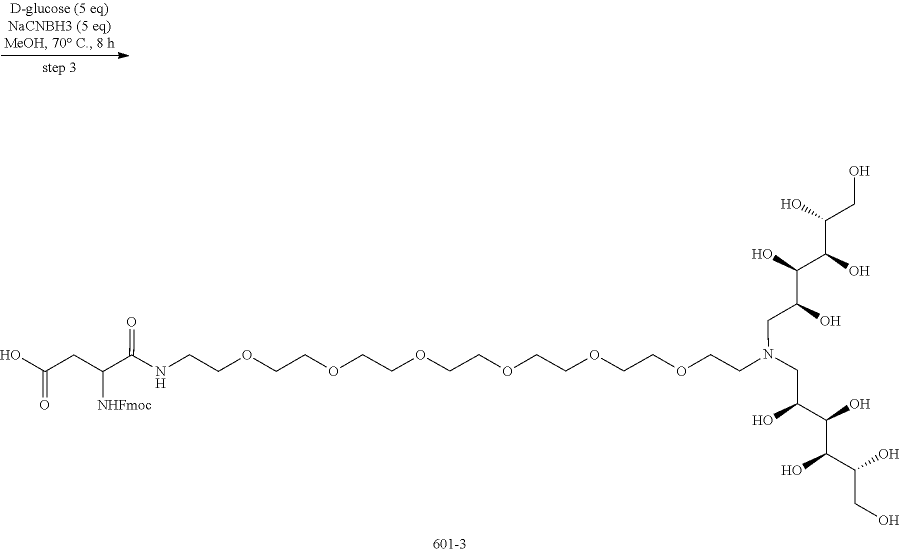

A solution of 601-2 (271 mg, 0.410 mmol) and D-Glucose (369 mg, 2.048 mmol) in anhydrous Methanol (45 mL) was heated at 50° C. for 30 min, then NaCNBH₃ (129 mg, 2.053 mmol) was added. The resulting solution was stirred for another 8 hr at 70° C. until LCMS indicated all starting amine was disappeared and the mass of desired product was detected. Then the reaction solution was concentrated and purified by reverse phase liquid chromatography to give 601-3 (182 mg, 0.184 mmol, 44.88%) as a white solid. purity=90%-95%.

### Step 4

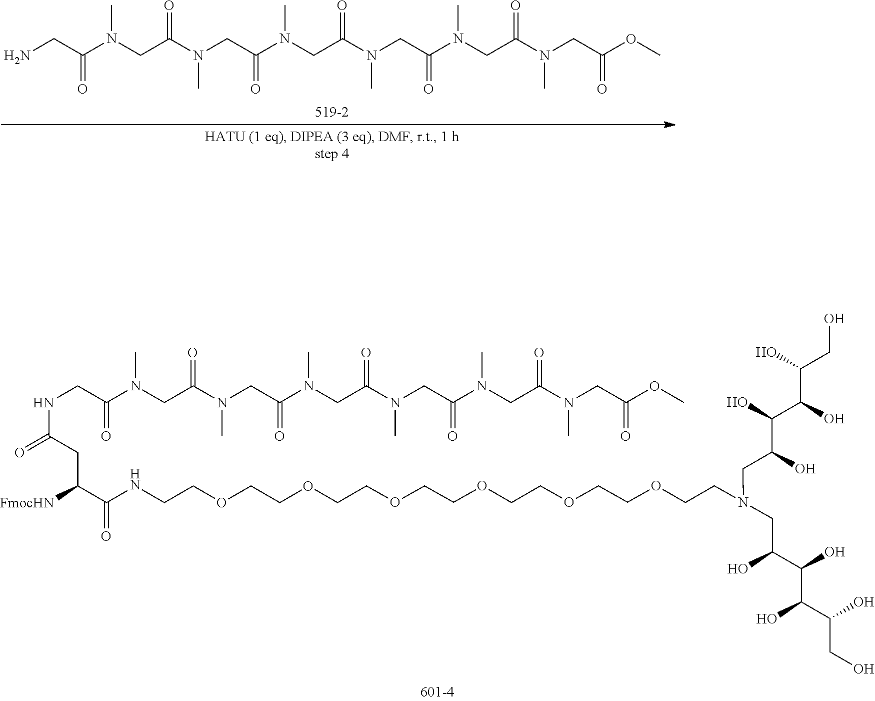

A solution of 601-3 (182 mg, 0.184 mmol), 519-2 (78 mg, 0.184 mmol) and HATU (70 mg, 0.184 mmol) in anhydrous DMF (5 mL) was stirred at room temperature for 5 min, then DIPEA (71 mg, 0.549 mmol) was added. The resulting solution was stirred for another 1 hr at r.t. until LCMS indicated complete reaction. The reaction solution was purified directly by reverse phase liquid chromatography to give 601-4 (156 mg, 0.105 mmol, 57.07%) as a white solid. purity=90%-95%.

### Step 5

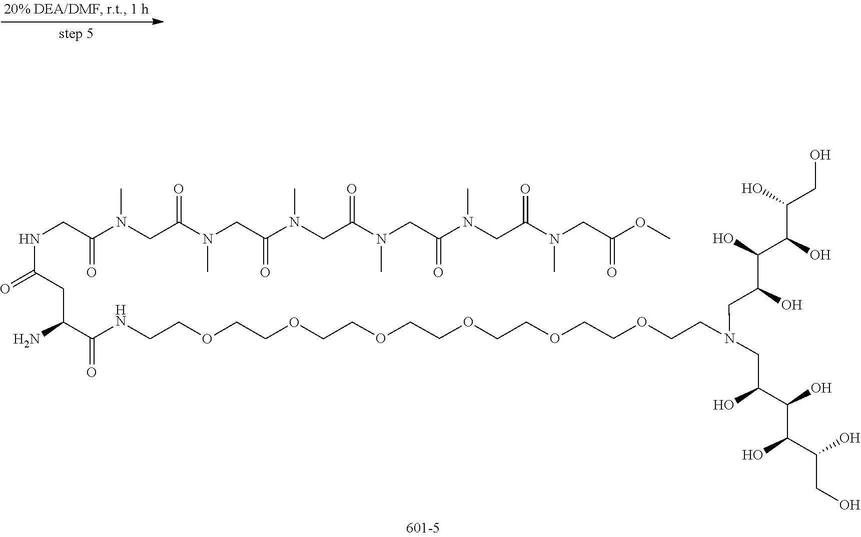 [SMILES: COC(=O)CN(C)C(=O)CN(C)C(=O)CN(C)C(=O)CN(C)C(=O)CN(C)C(=O)CN(C)C(=O)CNC(=O)C[C@H](N)C(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO]

A solution of 601-4 (156 mg, 0.105 mmol) and DEA (1 mL) in anhydrous DMF (4 mL) was stirred at room temperature for 1 hr until LCMS indicated all starting amine was disappeared and desired product was detected. Then the solution was concentrated to dryness to 601-5 (121 mg, 0.096 mmol, 91.43%) as colorless oil, used as such in the next step. purity=90%-95%.

### Step 6

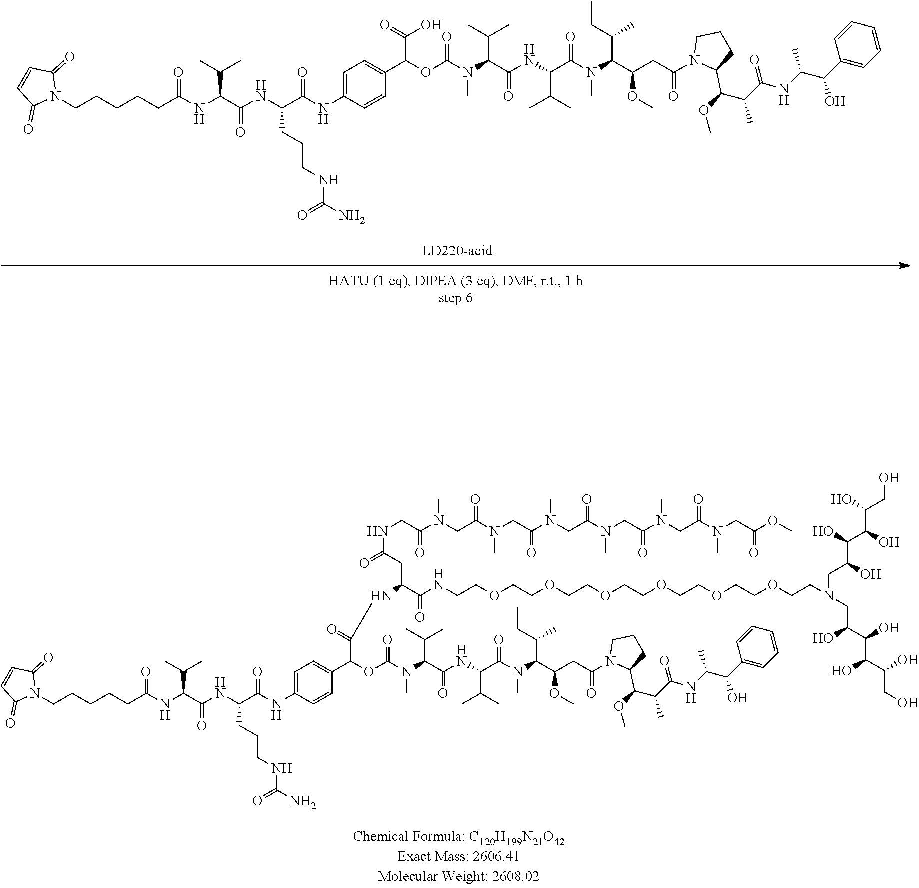

A solution of 601-5 (47 mg, 0.037 mmol), LD220-acid (50 mg, 0.037 mmol) and HATU (14 mg, 0.037 mmol) in anhydrous DMF (3 mL) was stirred at room temperature for 5 min, then DIPEA (14 mg, 0.108 mmol) was added. The resulting solution was stirred for another 1 hr at r.t. until LCMS indicated complete reaction. Then the reaction solution was purified by prep. HPLC to give LD601 (18 mg, 0.007 mmol, 18.92%) as a white solid. LCMS, m/z=1304.58 (M/2+H)⁺.

### Example 36: In Vivo Antitumor Activity for TAA1-Directed Conjugates of Drug-Linker 4

Anti-tumor activity of anti-TAA1 mAb conjugates of Drug-Linker 4 (Example 4) were evaluated in 4 CDX models for ovarian, lung and breast cancer in mice using the OVCAR-3 (Co-bioer, CBP60294, Ovarian cancer), PA-1 (Procell, CL-0411, Ovarian cancer), NCI-H292 (Co-bioer, CBP60187, Lung cancer) and MDA-MB-468 (Procell, CL-0290, Breast cancer) cell lines. Female NDG or BALB/c nude or NOD/SCID mice were inoculated subcutaneously at right flank with OVCAR-3 cells (1×10⁷ in 0.1 mL cell suspension), PA-1 cells (5×10⁶ in 0.1 mL cell suspension), NCI-H292 cells (5×10⁶ in 0.1 mL cell suspension), or MDA-MB-468 cells (5×10⁶ in 0.1 mL cell suspension) mixed with Matrigel (1:1). 4-24 days after tumor inoculation, mice with average tumor size of 128-156 mm³ were selected and assigned into 4-5 treatment groups using stratified randomization (n=6 per group) based upon their tumor volume. A single dose of the test articles was administered on day 0 after randomization (with randomization day defined as day 0) via intravenous injection.

The tumor size and body weight were measured twice a week. Tumor volume (measured in two dimensions using a caliper) was expressed in mm³ using the formula: V=0.5 a×b² where a and b represent the long and short diameter of the tumor, respectively. Tumor volume exceeding 2000 mm³ was regarded as endpoint. Animal body weight was monitored as an indirect assessment of toxicity. None of the mice showed significant weight loss in any of the treatment groups. DAR value of the ADC conjugates is delineated as in figure legends.

TABLE 1
Copy number of TAA1 on OVCAR-3, PA-1,
NCI-H292 and MDA-MB-468 tumor cells

| | Target Copy Number | |
|---|---|---|
| Cell line | (×10³ per cell) | Cancer type |
| OVCAR-3 | 10 | Ovarian |
| PA-1 | 63 | Ovarian |
| NCI-H292 | 22 | Lung adenocarcinoma |
| MDA-MB-468 | 41 | Triple negative breast |

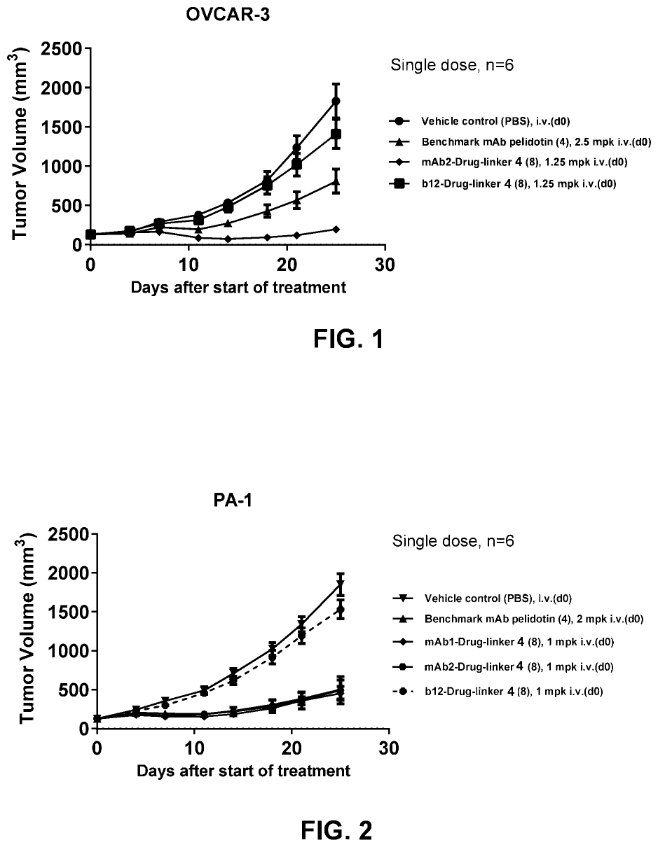

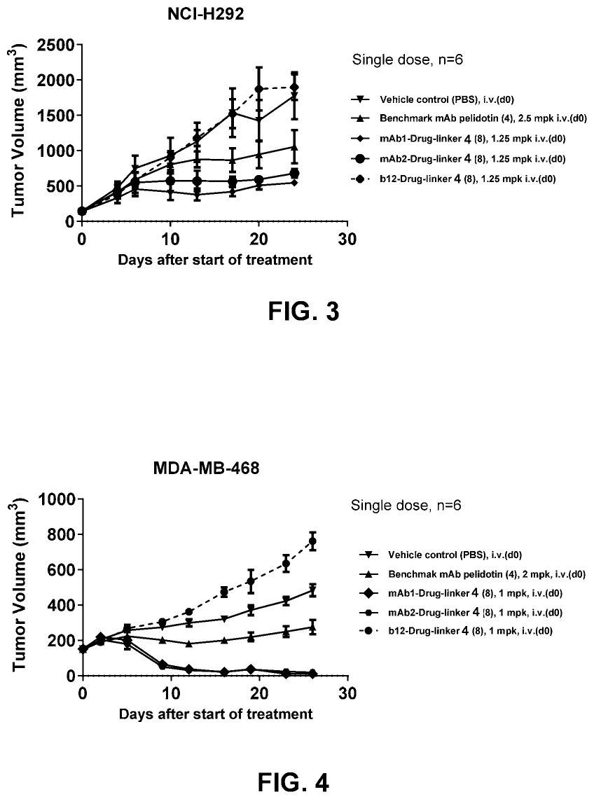

The in vivo studies data is shown in FIG. 1-4. There were no morbidity or death in the study duration. Anti-TAA1 mAbs-Drug-linker 4 (8) produced marked tumor growth inhibition compared to the vehicle control, and displayed significant stronger tumor growth inhibition than the Benchmark mAb pelidotin (4); the non-target control b12-Drug-linker 4 (8) did not elicit appreciable tumor growth inhibition compared to vehicle control.

The target (TAA1) copy number on OVCAR-3, PA-1, NCI-H292 and MDA-MB-468 tumor cells was tested by QIFKIT (DAKO, K0078). Briefly, Cells were labeled with primary mouse monoclonal antibody directed against the antigen of interest. The cells, Set-Up Beads, and Calibration Beads of the kit were then labeled in parallel with fluorescein-conjugated anti-mouse secondary antibody. The fluorescence correlated with the number of bound primary antibody molecules on the cells and on the beads. The samples were then analyzed on the flow cytometer and copy number calculated based on the equation derived from the calibration curve (Table 1). These tested tumor cell lines covered a related wide range of TAA1 expression levels.

### Example 37: In Vitro Activity for TAA2-Directed Conjugates of Drug-Linker 4

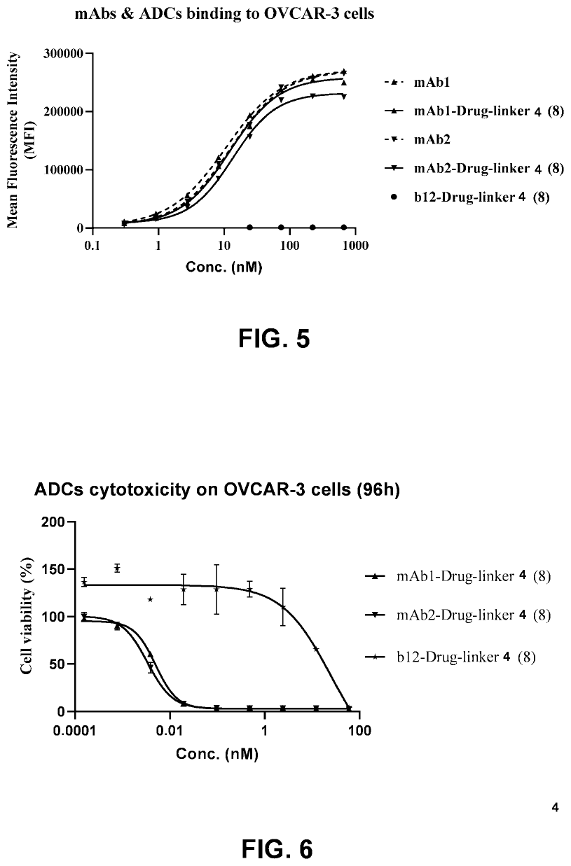

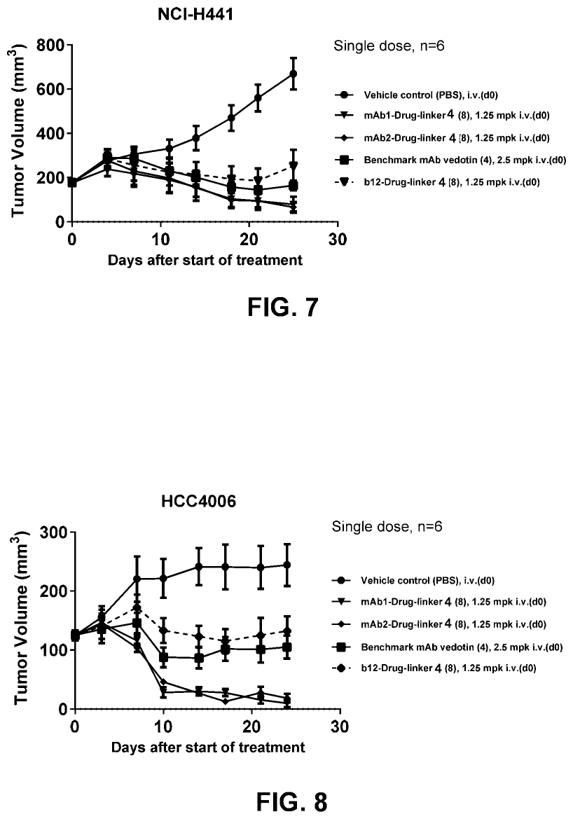

The binding activity of anti-TAA2 mAbs or anti-TAA2 mAbs Drug-linker 4 (8) was evaluated by Flow cytometric analysis (Beckman, Cytoflex) with target-expressing cell line OVCAR-3. Briefly, 3×10⁵ cells were seeded per well at 96-well v-bottomed plate and incubated with 100 μL of mAbs or ADCs in series dilutions. After 30 minutes of incubation at 4° C., cells were washed twice with PBS, stained with 100 μL of 1:200 diluted PE-conjugated anti human Fc in FACS buffer (1×PBS containing 1% BSA) and then incubated at 4° C. for 30 minutes. After that the cells were washed 2 times with PBS and analyzed by Flow cytometric analysis. The result is shown in FIG. 5.

The target (TAA2) copy number (Table 3) on OVCAR3 was tested by QIFKIT (DAKO, K0078). The study was carried out as described above.

The cytotoxicity of anti-TAA2 mAbs Drug-linker 4 (8) (Table 4) was evaluated in vitro. Briefly, one day prior to adding test article, cells were harvested and plated into 96-well solid white flat bottom plates. The next day cells were exposed to the test article at concentrations from 60.6 to 0.00016 nM for anti-TAA2 mAbs Drug-linker 4 (8) and negative control b12-Drug-linker 4 (8). Plates were incubated at 37° C. for 96 h. After that, 40 μL Cell-titre Glo (CTG) per well was added to the plates with luciferase readings collected at 5 minutes after and analyzed by Microplate readers. All readings were normalized as percentage of viable cells in the untreated control wells and the IC₅₀ values were calculated by Prism software.

TABLE 2
Copy number of TAA2 on OVCAR-3 tumor cell)

| | Target Copy Number | |
|---|---|---|
| Cell line | (×10³ per cell) | Tumor type |
| OVCAR-3 | 107 | Ovarian |

TABLE 3
The binding EC₅₀ of anti-TAA2 mAbs and its' conjugates

| | | mAb1- | | mAb2- | b12- |
|---|---|---|---|---|---|
| EC₅₀ (nM) | mAb1 | LD343 (8) | mAb2 | LD343 (8) | LD343 (8) |
| OVCAR-3 | 10.68 | 12.26 | 12.63 | 13.54 | NA |

TABLE 4
The cytotoxicity IC₅₀ of anti-TAA2 mAbs Drug-linker D (8)

| IC₅₀ (nM) | mAb1-LD343 (8) | mAb2-LD343 (8) | b12-LD343 (8) |
|---|---|---|---|
| OVCAR-3 | 0.00498 | 0.00336 | NA |

The cell OVCAR-3 used for assays has a high TAA2 expression level (Table 2). Anti-TAA2 mAbs and its' conjugates demonstrated strong binding activity to TAA2-expressing cell lines with the EC₅₀ in the range of 10-14 nM (Table 3). The binding activity of Anti-TAA2 mAbs was therefore not compromised after conjugation.

Anti-TAA2 mAbs Drug-linker D (8) produced strong cytotoxicity on TAA2-expressing OVCAR3 cell, while negative control b12-Drug-linker D (8) exerted no cytotoxicity on OVCAR3 cells (FIG. 6). The IC₅₀ of the tested ADCs is shown in Table 4.

### Example 38: In Vivo Antitumor Activity for TAA2-Directed Conjugates of Drug-Linker 4

The in vivo anti-tumor activity of Anti-TAA2 mAb conjugates (Anti-TAA2 mAb1 Drug-linker 4 (8) and Anti-TAA2 mAb2 Drug-linker 4 (8)) were evaluated in tumor cell lines NCI-H441 (Co-bioer, CBP60137, Lung cancer) and HCC4006 (ATCC, CRL-2871, Lung cancer) models.

NCI-H441 and HCC4006 tumor models were established by injecting 5×10⁶ cells suspended in 0.1 mL medium, mixed with Matrigel (1:1). 5-7 days after tumor inoculation, mice with average tumor size 124-178 mm³ were selected and assigned into 5 groups using stratified randomization (n=6 per group) based upon their tumor volumes. The treatments started after randomization and the mice were treated with a single (on day 0) intravenous injection of Anti-TAA2 mAbs Drug-linker 4 at 1.25 mg/kg, or Benchmark mAb vedotin (4) at 2.5 mg/kg, or b12-Drug-linker 4 (8) at 1.25 mg/kg. The tumor size and body weight were measured as described before. Animal body weight was monitored as an indirect measure of tolerability. No mice showed significant weight loss in any of the treatment groups. There were no morbidity and deaths during the treatment duration.

The target copy number (Table 5) was tested by QIFKIT (DAKO, K0078). The study was carried out as described above.

TABLE 5
Copy number of TAA2 on NCI-H441 and HCC4006 tumor cells

| | Target Copy Number | |
|---|---|---|
| Cell line | (×10³ per cell) | Cancer type |
| NCI-H441 | 5 | Lung adenocarcinoma |
| HCC-4006 | 56 | Lung adenocarcinoma |

Compared to vehicle control group, both the treatment groups with the test article (Anti-TAA2 mAbs Drug-linker 4 (8)) at single dose produced a significant antitumor activity in NCI-H441 and HCC4006 tumor models, and it was more potent than the Benchmark vedotin (4).

In Table 5, HCC-4006 has a high level TAA2 expression level while the target TAA2 expression level was low on NCI-H441.

### Example 39: In Vitro Activity for TAA3-Directed Conjugates of Drug-Linker 4

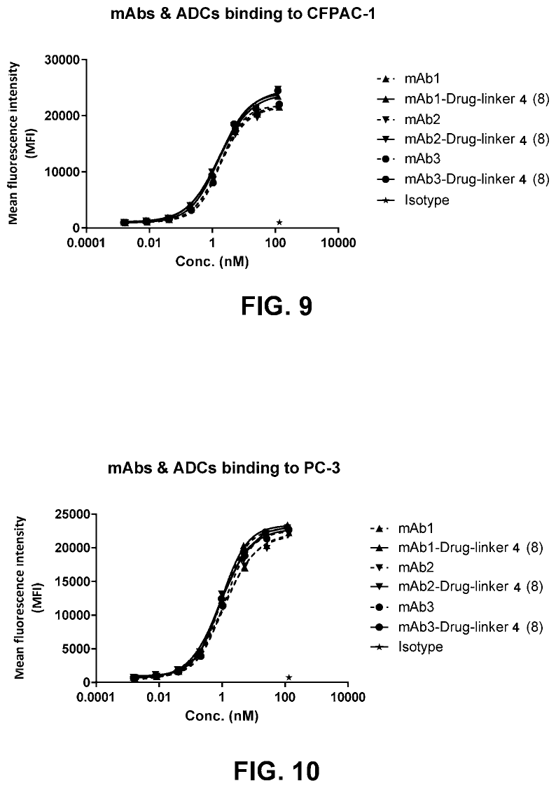

The binding activity of anti-TAA3 mAbs or anti-TAA3 mAbs Drug-linker 4 (8) and cytotoxicity of anti-TAA3 mAbs Drug-linker 4 (8) were evaluated with target-expressing cell lines CFPAC-1 and PC-3. The assays were conducted as described before. The results are shown in FIG. 9-12. The binding EC₅₀ of mAbs or ADCs and the cytotoxicity IC₅₀ of ADCs are shown in Table 7 and Table 8, respectively.

The target (TAA3) copy number on CFPAC-1 and PC-3 was tested by QIFKIT (DAKO, K0078), the assay was conducted as described before, the results are shown in Table 6.

TABLE 6
Copy number of TAA3 on CFPAC-1 and PC-3 tumor cells

| | Target Copy Number | |
|---|---|---|
| Cell line | (×10³ per cell) | Tumor type |
| CFPAC-1 | 135 | pancreatic |
| PC-3 | 21 | prostate |

TABLE 7
The binding EC₅₀ of anti-TAA3 mAbs and its conjugates

| EC₅₀ (nM) | | mAb1 | mAb2 | mAb3 |
|---|---|---|---|---|
| CFPAC-1 | mAb | 1.66 | 1.76 | 1.83 |
| | ADC | 1.68 | 1.68 | 1.69 |
| PC-3 | mAb | 1.11 | 1.16 | 1.15 |
| | ADC | 0.93 | 0.87 | 0.92 |

TABLE 8
The cytotoxicity IC₅₀ of anti-TAA3 mAbs Drug-linker 4 (8)

| | mAb1- | mAb2- | mAb3- | b12- |
|---|---|---|---|---|
| IC₅₀ (nM) | LD343 (8) | LD343 (8) | LD343 (8) | LD343 (8) |
| CFPAC-1 | 0.09 | 0.06 | 0.04 | ~67.85 |
| PC-3 | 0.09 | 0.14 | 0.02 | ~14.07 |

The expression level of the cell lines (CFPAC-1 and PC-3) used for the experiments covered both high and low expression level (Table 6).

Anti-TAA3 mAbs and anti-TAA3 mAbs Drug-linker 4 (8) demonstrated strong binding activity to TAA3-expressing cell lines with the EC₅₀ (Table 7) in the range of 0.87-1.83 nM. The binding activity of anti-TAA3 mAbs was therefore not compromised after conjugation.

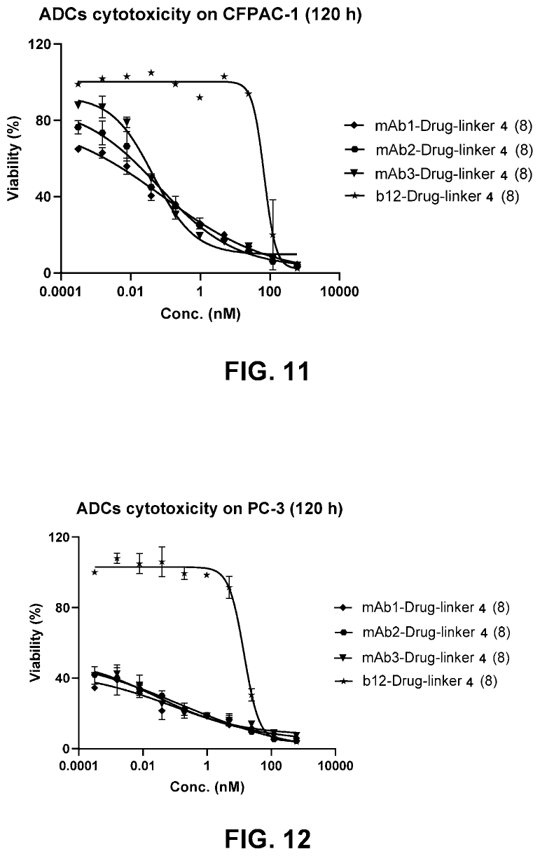

Anti-TAA3 mAbs Drug-linker 4 (8) produced strong cytotoxicity on TAA3-expressing CFPAC-1 and PC-3 cells, while negative control b12-Drug-linker 4 (8) exerted no cytotoxicity on these tested cells (FIG. 11-12). The IC₅₀ of the tested ADCs was in the range of 0.02-0.14 nM, the data is shown in Table 8.

### Example 40: In Vivo Antitumor Activity for TAA3-Directed Conjugates of Drug-Linker 4

The in vivo anti-tumor activity of anti-TAA3 mAbs Drug-linker 4 (8) were evaluated in cell line models which represented high and low target expression level, CFPAC-1 (Co-bioer, CBP60665, Pancreatic cancer) and PC-3 (Co-bioer, CBP60343, Prostate cancer) cells.

The study method was carried out as described before. Briefly, CFPAC-1 and PC-3 tumor model were established by injecting 5×10⁶ cells suspended in Matrigel/medium (1:1). 4-6 days after tumor inoculation, mice with average tumor size of 118-138 mm³ were selected and assigned into 4 or 5 groups for each model using stratified randomization (n=5-6 per group) based upon their tumor volumes. The treatments started after randomization and were in a single dose (on day 0) via intravenous injection of the test articles at 1.25 mg/kg. The tumor size and body weight were measured as described before. Animal body weight was monitored as an indirect measure of tolerability. No mice showed significant weight loss in any of the treatment groups. There were no morbidity and deaths during the treatment duration.

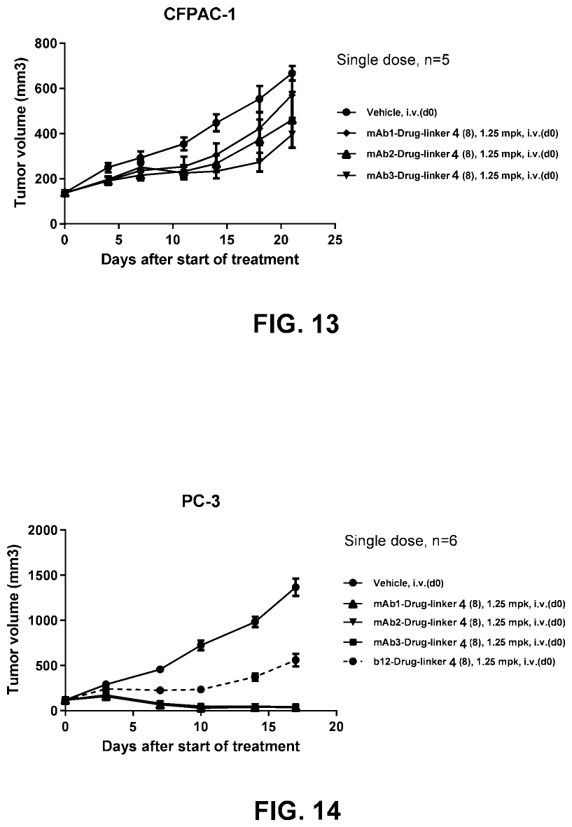

FIGS. 13 and 14 shows graphs of tumor volume versus treatment period for different ADCs in CFPAC-1 and PC-3 respectively. Compared to vehicle control, treatment with anti-TAA3 mAbs Drug-Linker 4 (8) at single dose demonstrated significant antitumor activity in the PC-3 tumor model, and modest activity in the CFPAC-1 model.

All of the U.S. patents, U.S. patent application publications, U.S. patent applications, foreign patents, foreign patent applications and non-patent publications referred to in this specification or the attached Application Data Sheet are incorporated herein by reference, in their entirety to the extent not inconsistent with the present description.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A Linker compound, or a stereoisomer or salt thereof, comprising:
(a) a Linker unit having from 1 to 4 attachment sites for a Drug unit and having one of the following structures (i) or (ii): 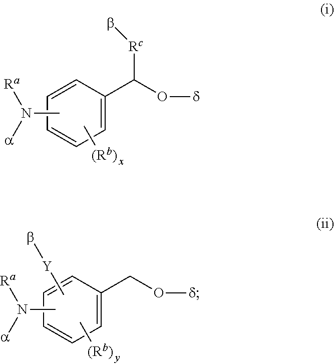
(b) at least one Polar group comprising a Polymer unit, optionally a Sugar unit, optionally a Carboxyl unit, and combinations thereof; and
(c) optionally a Stretcher group having an attachment site for a Targeting group;
wherein:
α— is an attachment site to an enzyme-cleavable group;
β—is an attachment site to the at least one Polar group;
δ—is H, an attachment site to at least one of the Drug units, or an attachment site to a linking group attached to the at least one of the Drug units;
the Polymer unit comprises a polyamide, a polyether, or a combination thereof, wherein the polyether comprises a hydroxyl group, a polyhydroxyl group, a sugar group, a carboxyl group, or combinations thereof;
each R^{a} independently is H or C₁-C₆ alkyl;
each R^{b} independently is halo, C₁₋₆ alkyl, an attachment site to at least one of the Drug units, or an attachment site to at least one of the Polar groups;
x is 0, 1, 2, 3, or 4;
y is 0, 1, 2, or 3;
R^{c} is a bond, —C(O)—, —S(O)—, —SO₂—, C₁₋₆ alkylene, C₁₋₆ alkynylene, triazolyl, or combinations thereof; and
Y is a bond, —O—, —S—, —N(R^{a})—, —C(O)—, —S(O)—, —SO₂—C₁-C₆ alkylene, C₁-C₆ alkenylene, C₁-C₆ alkynylene, a group containing triazolyl, or combinations thereof.

2. The Linker compound of claim 1, wherein the Linker unit has one of the following structures (i-a), (ii-a), or (iii-a): 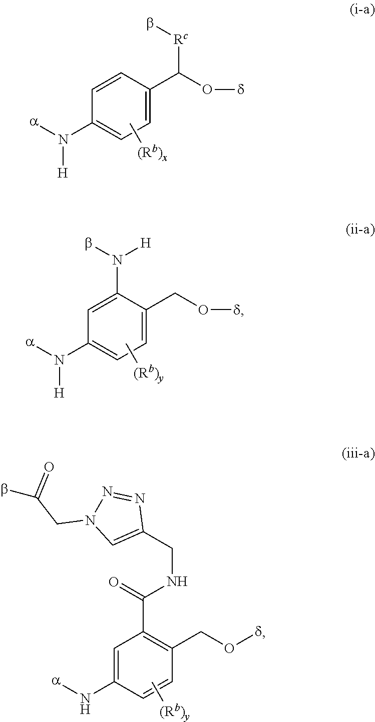
or a stereoisomer or salt thereof.

3. The Linker compound of claim 1, wherein the Linker unit has one of the following structures (i-b), (i-c), (i-d), (i-e), or (i-f): 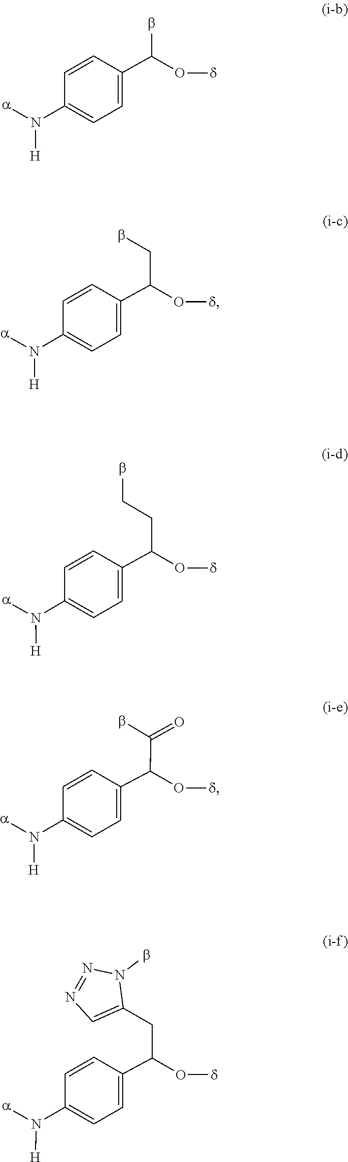
or a stereoisomer or salt thereof.

4. The Linker compound of claim 1, wherein the Linker unit has the following structure (ii-b) or (iii-b): 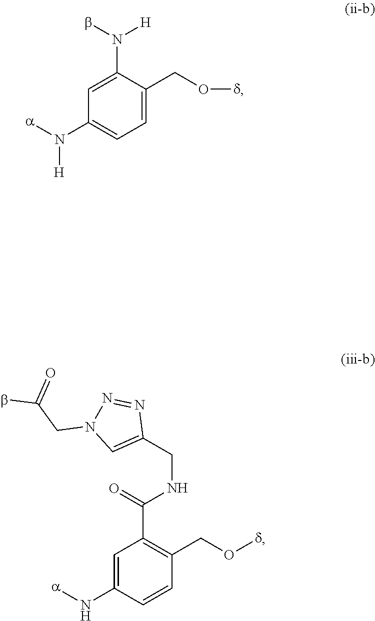
or a stereoisomer or salt thereof.

5. The Linker compound of any one of claims 1-4, wherein the at least one Polar group comprises at least one Sugar unit having the following formula: 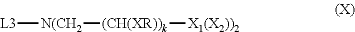
or a stereoisomer or salt thereof, wherein:
each X is independently selected from NH and O;
each R is independently selected from hydrogen, acetyl, a monosaccharide, a disaccharide, and a polysaccharide;
each X₁ is independently selected from CH₂ and C(O);
each X₂ is independently selected from H, OH and OR;
k is 1 to 10; and
L3 is a point of attachment to a remainder of the Polar group.

6. The Linker compound any one of claims 1-4, wherein the at least one Sugar unit has one of the following structures (XII) or (XIII): 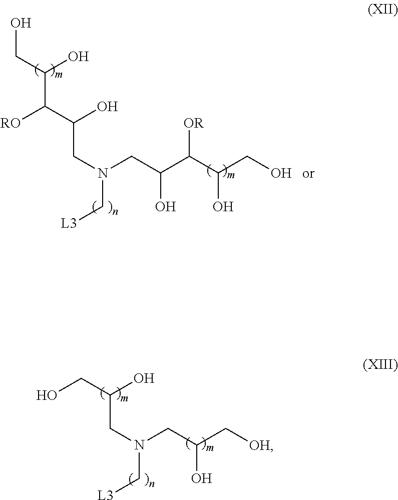
or a stereoisomer or salt thereof, wherein:
each R is independently selected from hydrogen, a monosaccharide, a disaccharide and a polysaccharide;
m is 1 to 8; and
n is 0 to 4.

7. The Linker compound of any one of claims 1-6, comprising a Polar group having a formula selected from: 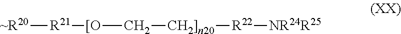
or a stereoisomer a salt thereof, wherein:
R²⁰ is an attachment group to site R^{b}, or to the enzyme-cleavable group;
R²¹ and R²² are each, independently, a bond or C₁-C₃ alkylene;
R²⁴ and R²⁵ are each independently selected from a H; polyhydroxyl group; substituted polyhydroxyl group; —C(O)-polyhydroxyl group; substituted —C(O)-polyhydroxyl group; substituted C₁-C₈ alkyl; substituted —C(O)—C₁-C₈ alkyl; a chelator; and —C(O)—R²⁸, where R²⁸ is a Sugar unit of formula (XII) or (XIII), provided that R²⁴ and R²⁵ are not both H; and
n20 is 2 to 26; or 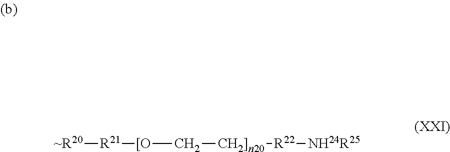
or a stereoisomer or salt thereof, wherein:
R²⁰ is an attachment group to site R^{b}, or to the enzyme-cleavable group;
R²¹ and R²² are each, independently, a bond or C₁-C₃ alkylene;
one of R²⁴ and R²⁵ is selected from a H; polyhydroxyl group; substituted polyhydroxyl group; —C(O)-polyhydroxyl group; substituted —C(O)-polyhydroxyl group; substituted C₁-C₈ alkyl; substituted —C(O)—C₁-C₈ alkyl; a chelator; and —C(O)—R²⁸, where R²⁸ is a Sugar unit of formula (XII) or (XIII); and the other of R²⁴ and R²⁵ is a polyethylene glycol, optionally having 1 to 24 ethylene glycol subunits; and
n20 is 2 to 26; or 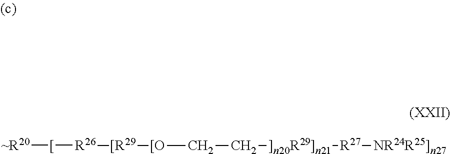
or a stereoisomer or salt thereof, wherein:
R²⁰ is an attachment group to site R^{b}, or to the enzyme-cleavable group;
R²⁶ and R²⁷ are each optional and are, independently, selected from a bond, C₁-C₁₂ alkylene, —NH—C₁-C₁₂ alkylene, —C₁-C₁₂ alkylene-NH—, —C₁-C₁₂ alkylene-N(CH₃)—, —C(O)—C₁-C₁₂ alkylene, —C₁-C₁₂ alkylene-C(O)—, —NH—C₁-C₁₂ alkylene-C(O)— and —C(O)—C₁-C₁₂ alkylene-NH—;
one of R²⁴ and R²⁵ is selected from a H; polyhydroxyl group; substituted polyhydroxyl group; —C(O)-polyhydroxyl group; substituted —C(O)-polyhydroxyl group; substituted C₁-C₈ alkyl; substituted —C(O)—C₁-C₈ alkyl; a chelator; and —C(O)—R²⁸, where R²⁸ is a Sugar unit of formula (XII) or (XIII); and the other of R²⁴ and R²⁵ is selected from H; polyhydroxyl group; substituted polyhydroxyl group; —C(O)-polyhydroxyl group; substituted —C(O)-polyhydroxyl group, where R²⁸ is a Sugar unit of formula (XII) or (XIII); and polyethylene glycol, optionally having 1 to 24 ethylene glycol subunits, provided that R²⁴ and R²⁵ are not both H;
each R²⁹ is optional and independently selected from —C(O)—, —NH—, —C(O)—C₁-C₆ alkylene-, —NH—C₁-C₆ alkylene-, —C₁-C₆ alkylene-NH—, —C₁-C₆ alkylene-C(O)—, —NH(CO)—C₁-C₆alkylene-, —N(CH₃)—(CO)—C₁-C₆alkylene-, —NH(CO)NH—, and triazole;
n20 is 2 to 26;
n21 is 1 to 4; and
n27 is 1 to 4, or 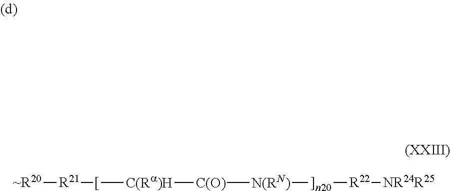
or a stereoisomer or salt thereof, wherein:
R²⁰ is an attachment group to site R^{b}, or to the enzyme-cleavable group;
R²¹ is a bond or C₁-C₃ alkylene,
—C₁-C₃alkylene-[O—CH₂—CH₂-]ₙ₂₀, —[CH₂—CH₂—O]ₙ₂₀—C₁-C₃alkylene- or —C₁-C₃alkylene-[O—CH₂—CH₂-]ₙ₂₀-C(O)—;
R²² is C₁-C₃ alkylene, —C₁-C₃alkylene-[O—CH₂—CH₂-]ₙ₂₀, —[CH₂—CH₂—O]ₙ₂₀—C₁-C₃alkylene- or —C₁-C₃alkylene-[O—CH₂—CH₂-]ₙ₂₀-C(O)—;
each R^{α} is independently H or —R²²—NR²⁴R²⁵;
each R^{N} is independently H, C₁-C₆ alkyl or —R²²—NR²⁴R²⁵;
R²⁴ and R²⁵ are each independently selected from a H; polyhydroxyl group; substituted polyhydroxyl group; —C(O)-polyhydroxyl group; substituted —C(O)-polyhydroxyl group; substituted C₁-C₈ alkyl; substituted —C(O)—C₁-C₈ alkyl; a chelator; and —C(O)—R²⁸, where R²⁸ is a Sugar unit of formula (XII) or (XIII), provided that R²⁴ and R²⁵ are not both H; and
each n20 is independently 2 to 26, or 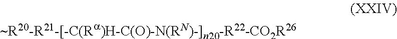
or a stereoisomer or salt thereof, wherein:
R²⁰ is an attachment group to site R^{b}, or to the enzyme-cleavable group;
R²¹ and R²² are each, independently, a bond, C₁-C₃ alkylene, or —C₁-C₃alkylene[O—CH₂—CH₂-]ₙ₂₀;
each R^{α} is independently H or —R²²—NR²⁴R²⁵;
each R^{N} is independently H, C₁-C₆ alkyl or —R²²—NR²⁴R²⁵;
R²⁴ and R²⁵ are each independently selected from a H; polyhydroxyl group; substituted polyhydroxyl group; —C(O)-polyhydroxyl group; substituted —C(O)-polyhydroxyl group; substituted C₁-C₈ alkyl; substituted —C(O)—C₁-C₈ alkyl; a chelator; and —C(O)—R²⁸, where R²⁸ is a Sugar unit of formula (XII) or (XIII), provided that R²⁴ and R²⁵ are not both H;
R²⁶ is H or C₁-C₄ alkyl; and
each n20 is independently 2 to 26,
with the proviso that at least one R^{a} or R^{N} is —R²²—NR²⁴R²⁵; or 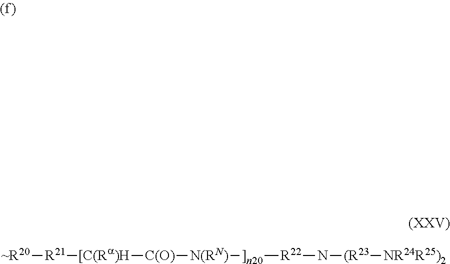
or a stereoisomer or salt thereof, wherein:
R²⁰ is an attachment group to site R^{b}, or to the enzyme-cleavable group;
R²¹ and R²² are each, independently, a bond, C₁-C₃ alkylene, or —C₁-C₃alkylene-[O—CH₂—CH₂-]ₙ₂₀;
each R^{α} is independently H or —R²²—NR²⁴R²⁵;
each R^{N} is independently H or C₁-C₆ alkyl;
each R²³ is independently C₁-C₆ alkylene;
R²⁴ and R²⁵ are each independently selected from a H; polyhydroxyl group; substituted polyhydroxyl group; —C(O)-polyhydroxyl group; substituted —C(O)-polyhydroxyl group; substituted C₁-C₈ alkyl; substituted —C(O)—C₁-C₈ alkyl; a chelator; and —C(O)—R²⁸, where R²⁸ is a Sugar unit of formula (XII) or (XIII), provided that R²⁴ and R²⁵ are not both H; and
each n20 is independently 2 to 26.

8. The Linker compound of claim 7, wherein R²⁴ and R²⁵ are each independently selected from H and a polyhydroxyl group, provided that R²⁴ and R²⁵ are not both H.

9. The Linker compound of claim 7 or 8, wherein the polyhydroxyl group is a linear monosaccharide, optionally selected from a C6 or C5 sugar, a sugar acid, and an amino sugar.

10. The Linker compound of claim 9, wherein:
the C6 or C5 sugar is selected from glucose, ribose, galactose, mannose, arabinose, 2-deoxyglucose, glyceraldehyde, erythrose, threose, xylose, lyxose, allose, altrose, gulose, idose, talose, aldose, and ketose;
the sugar acid is selected from gluconic acid, aldonic acid, uronic acid, and ulosonic acid; or
the amino sugar is selected from glucosamine, N-acetyl glucosamine, galactosamine, and N-acetyl galactosamine.

11. The Linker compound of any one of claims 7 to 10, comprising a Polar group selected from the following, or a stereoisomer or salt thereof: 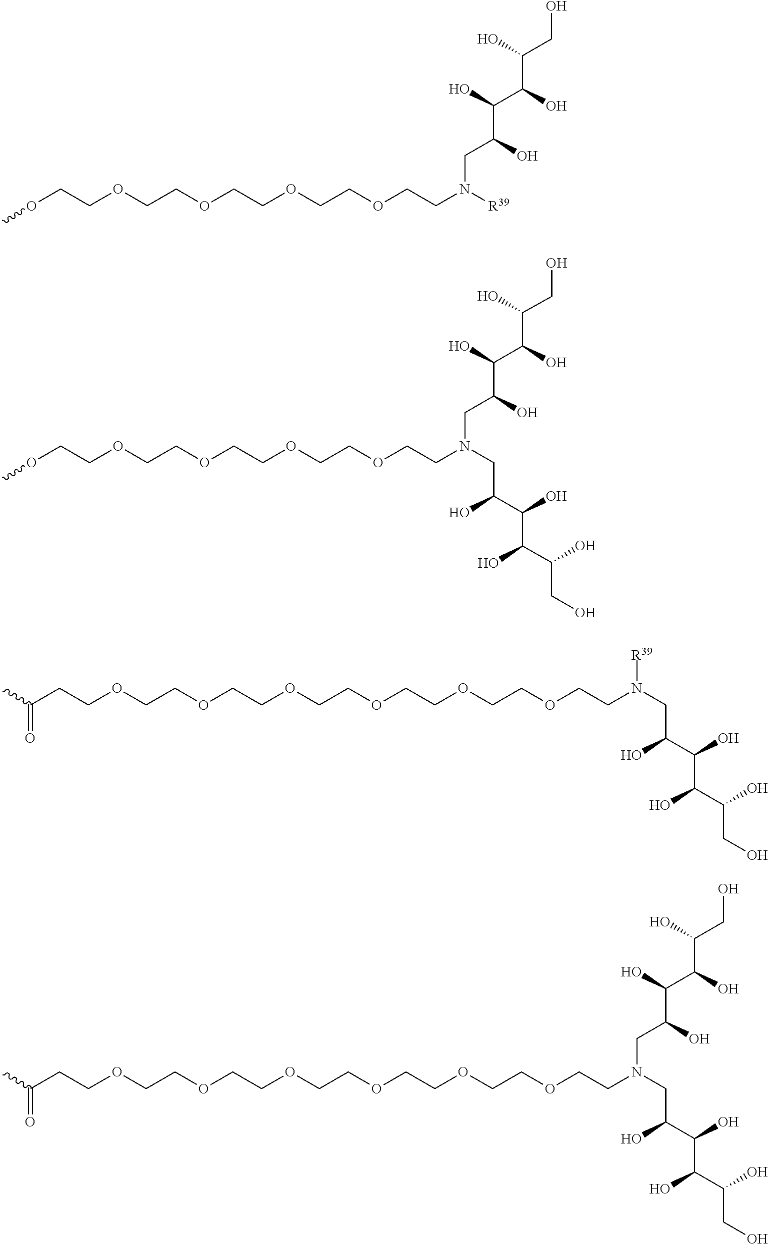 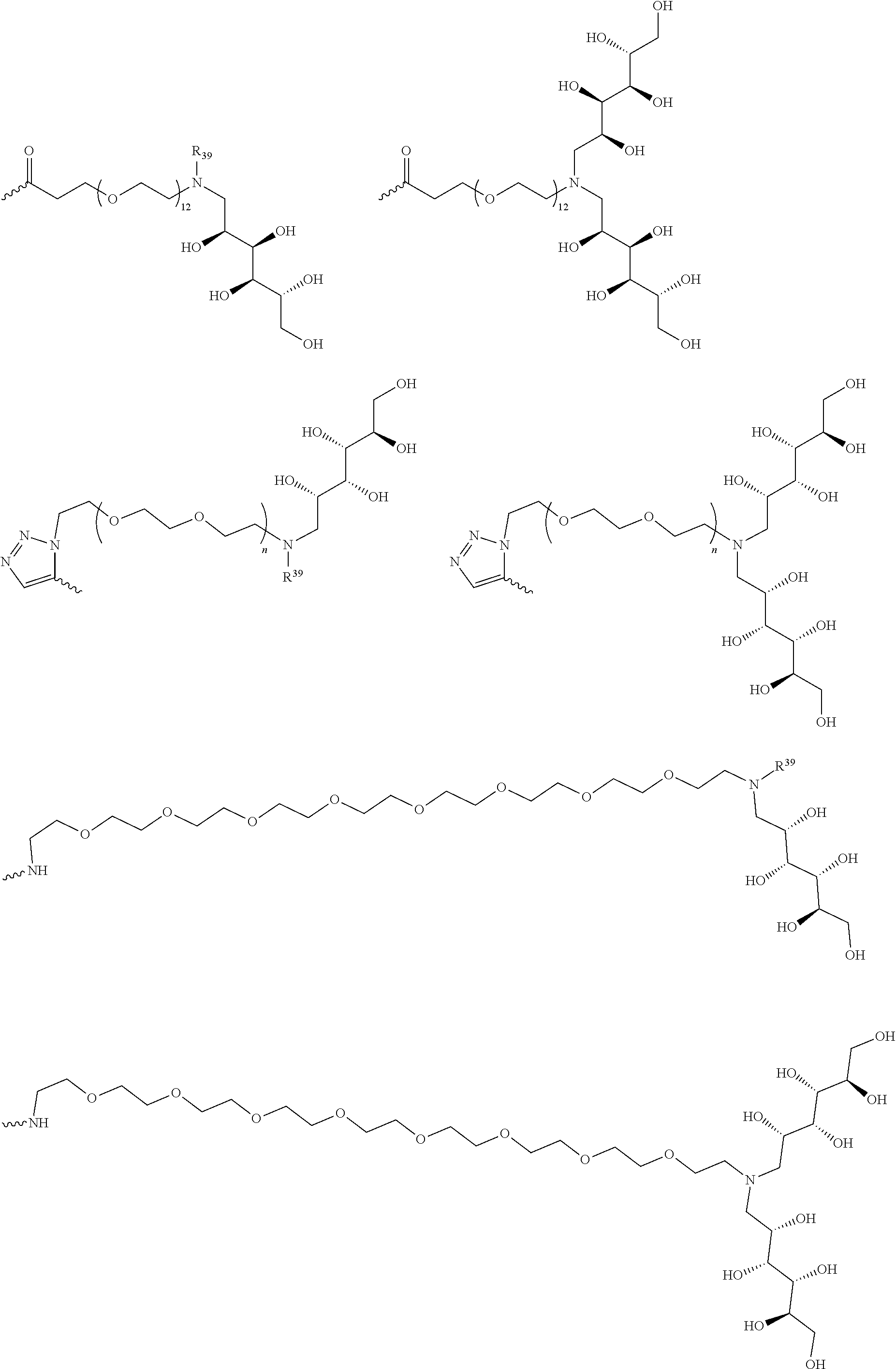 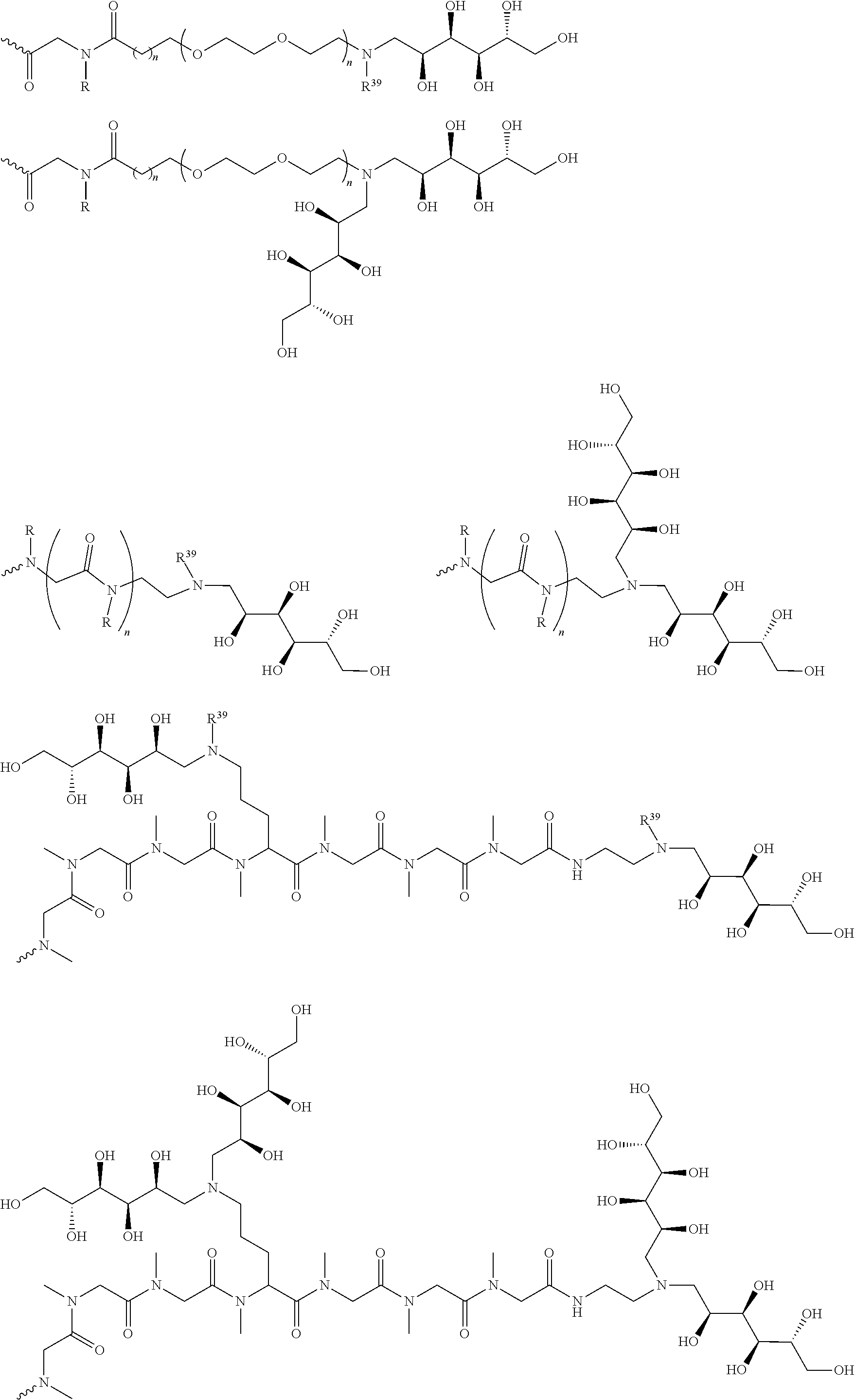 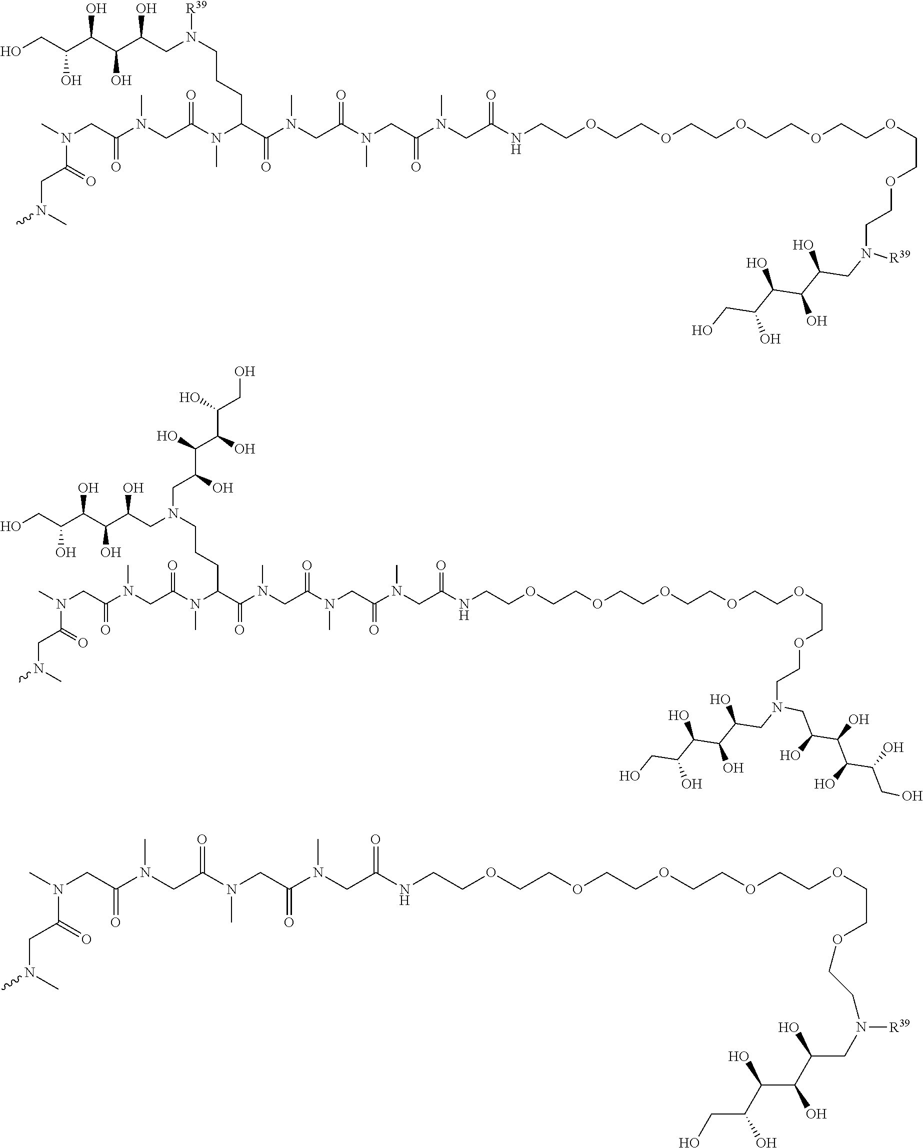 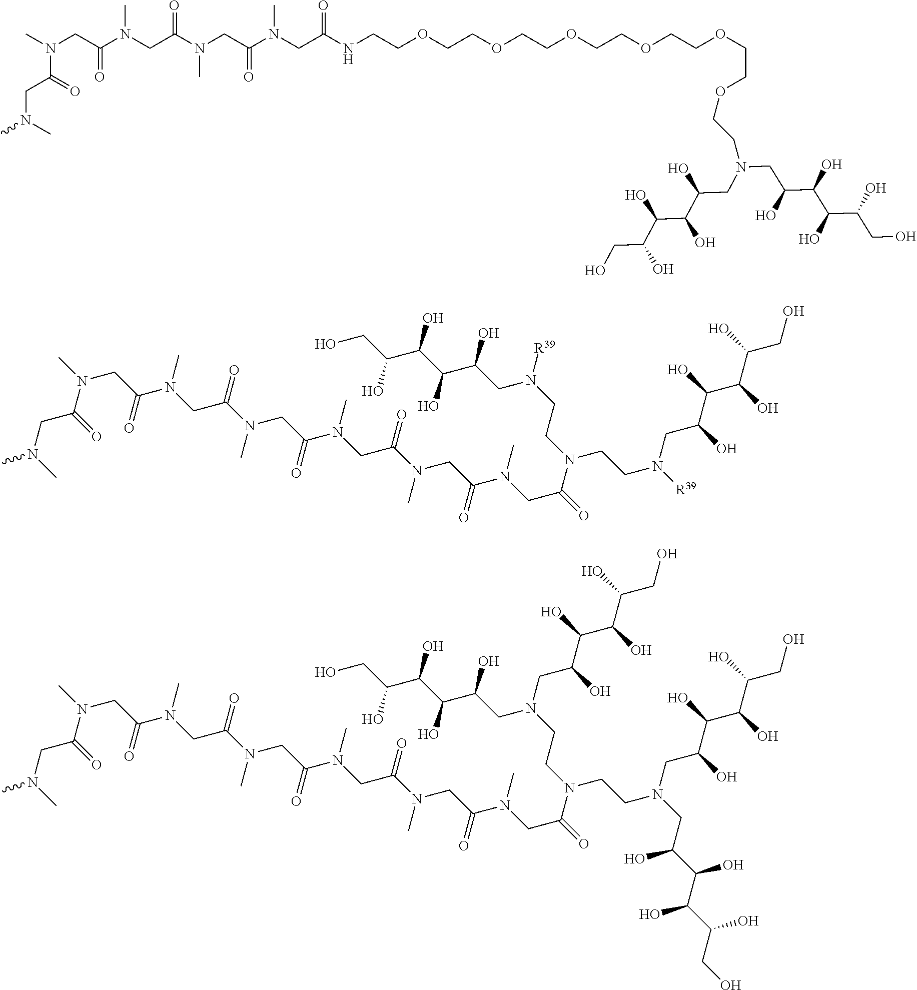 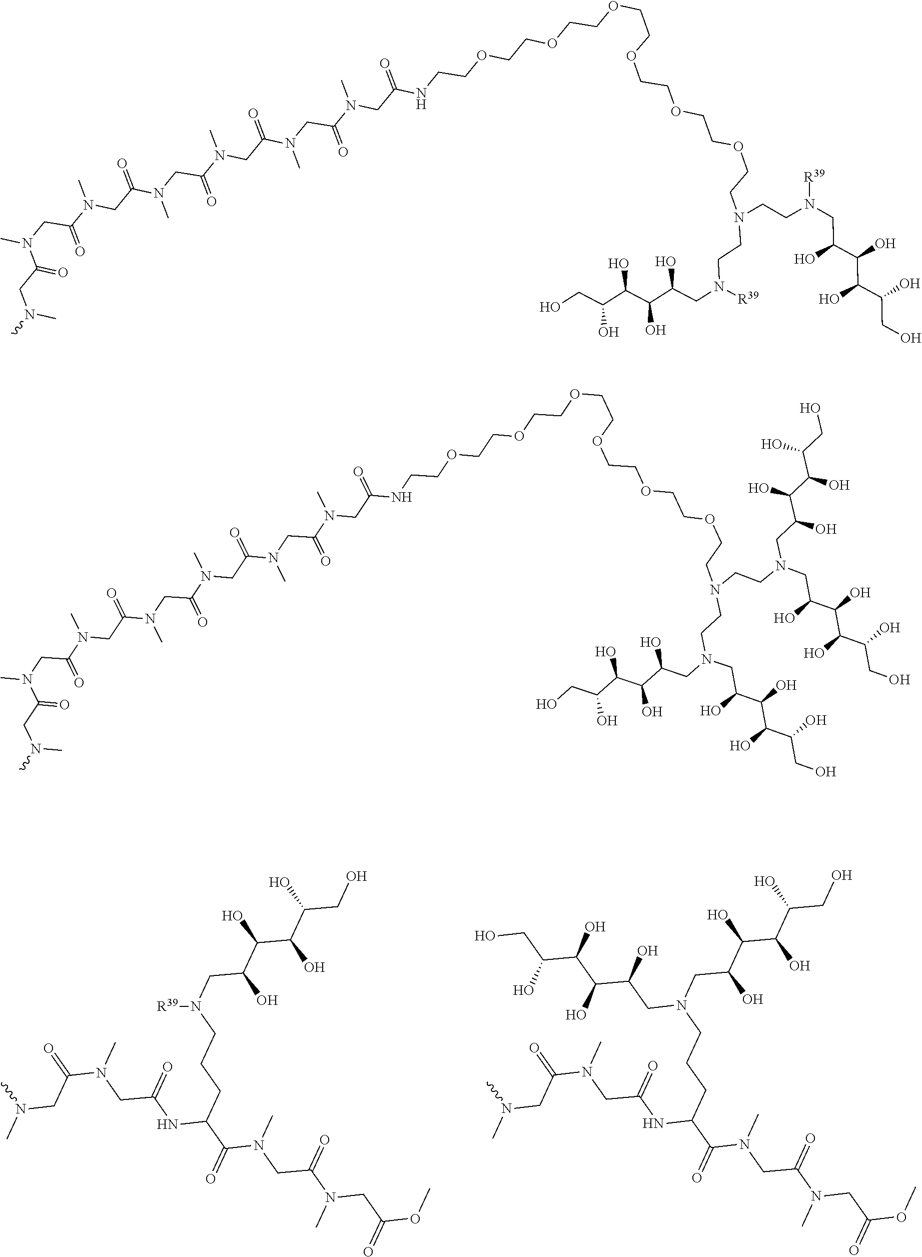 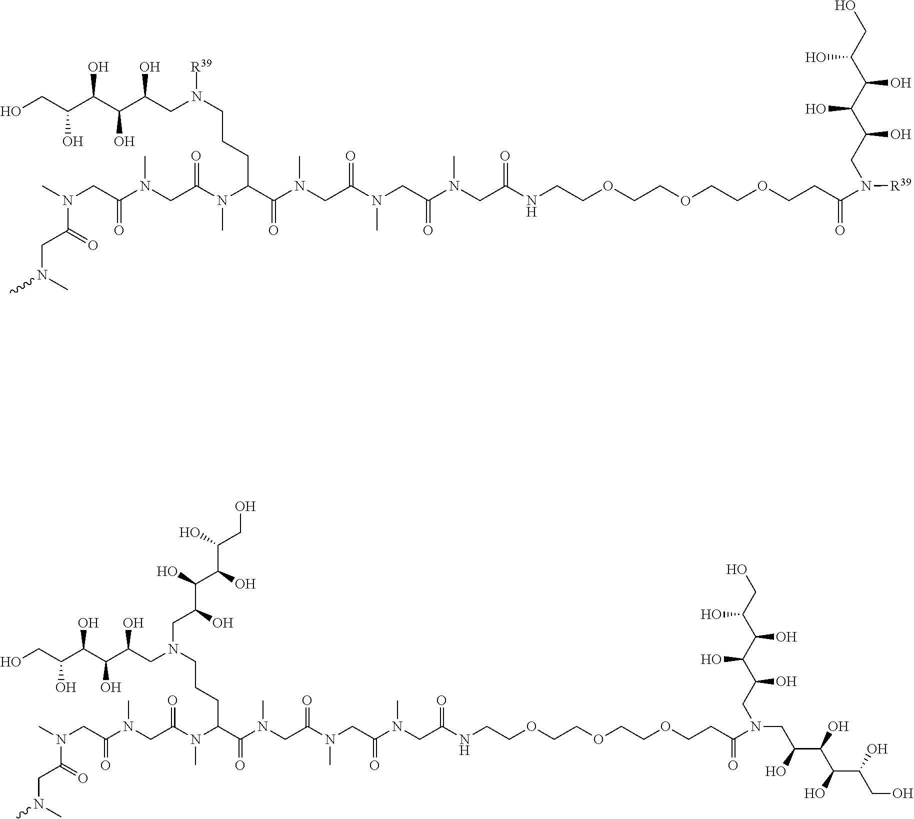 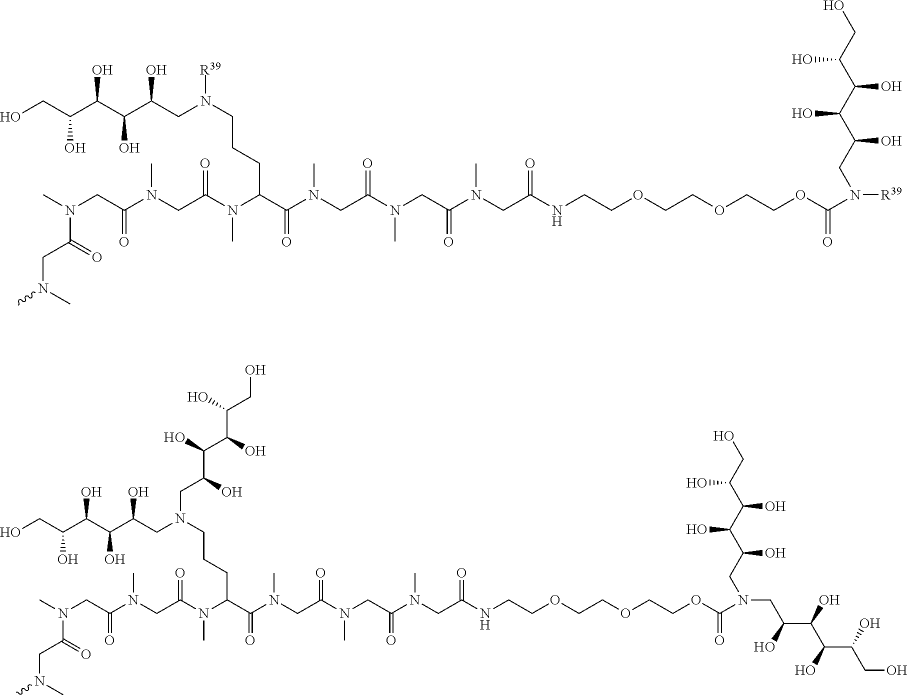 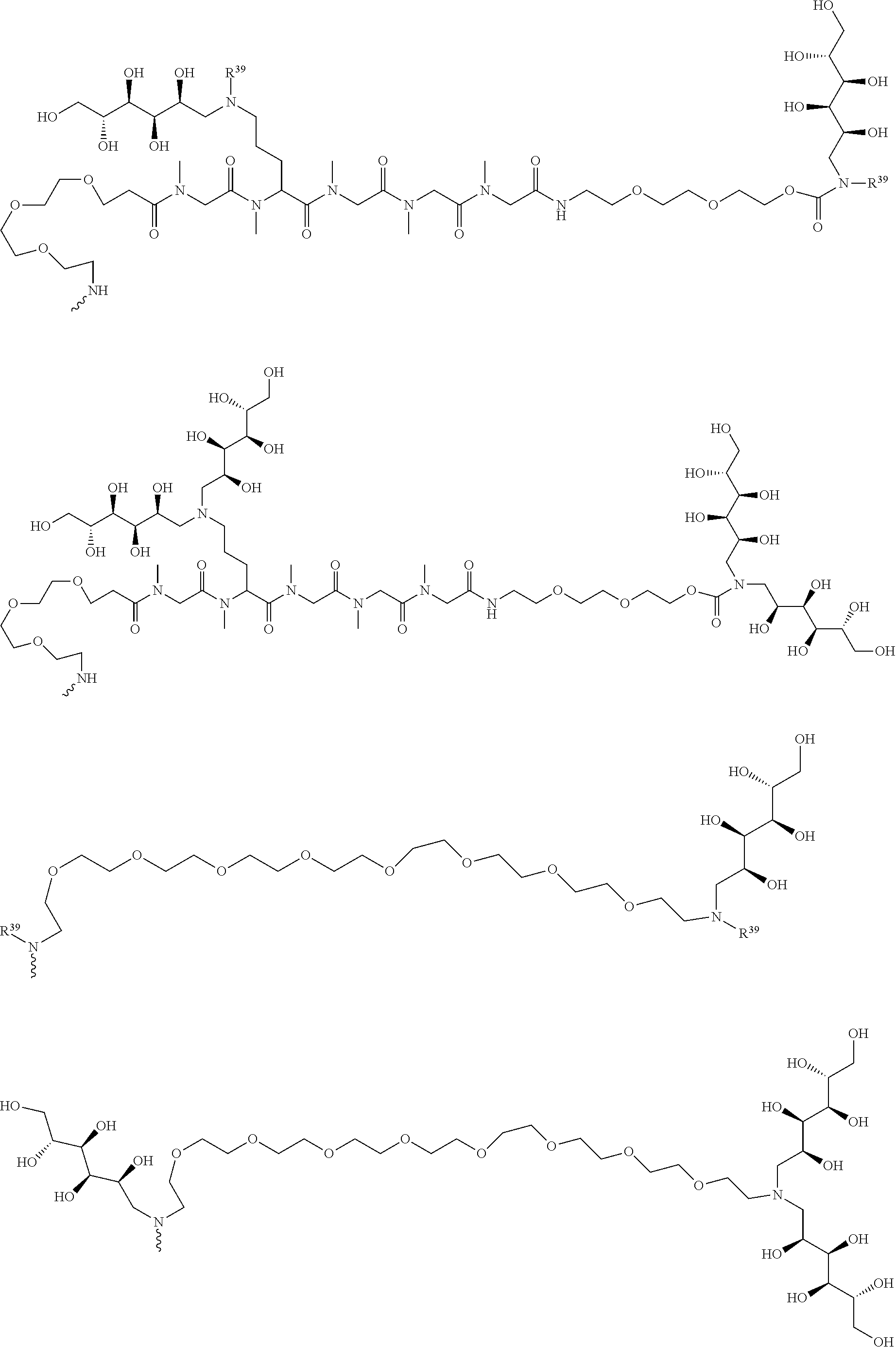 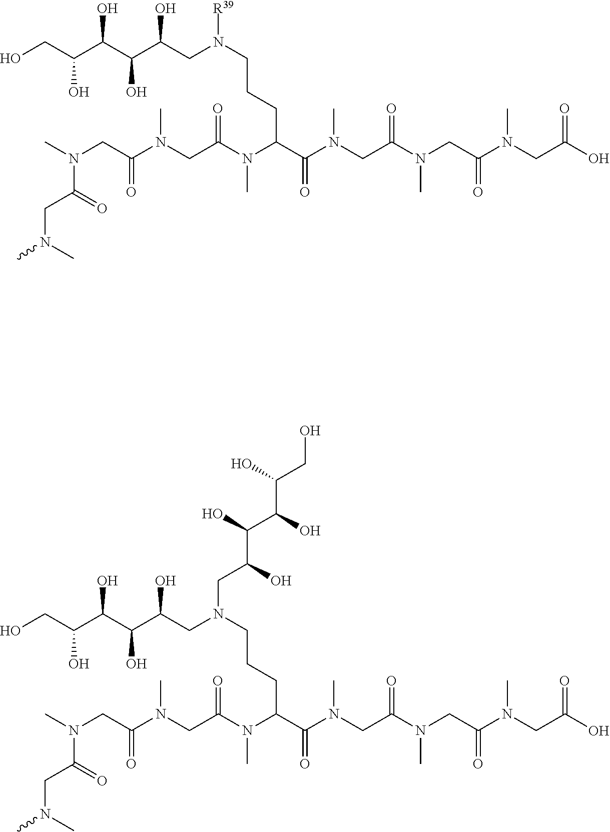
wherein each R is independently H or alkyl; each R³⁹ is independently selected from H, a linear monosaccharide and polyethylene glycol, optionally having from 1 to 24 ethylene glycol subunits; each n independently is 1-12; and the wavy line is an attachment site to R^{b}, or to the enzyme-cleavable group.

12. The Linker compound of claim 7 or 8, wherein one of R²⁴ and R²⁵ is a linear monosaccharide and the other is a cyclic monosaccharide.

13. The Linker compound of claim 12, comprising a Polar group selected from the following, or a stereoisomer or salt thereof: 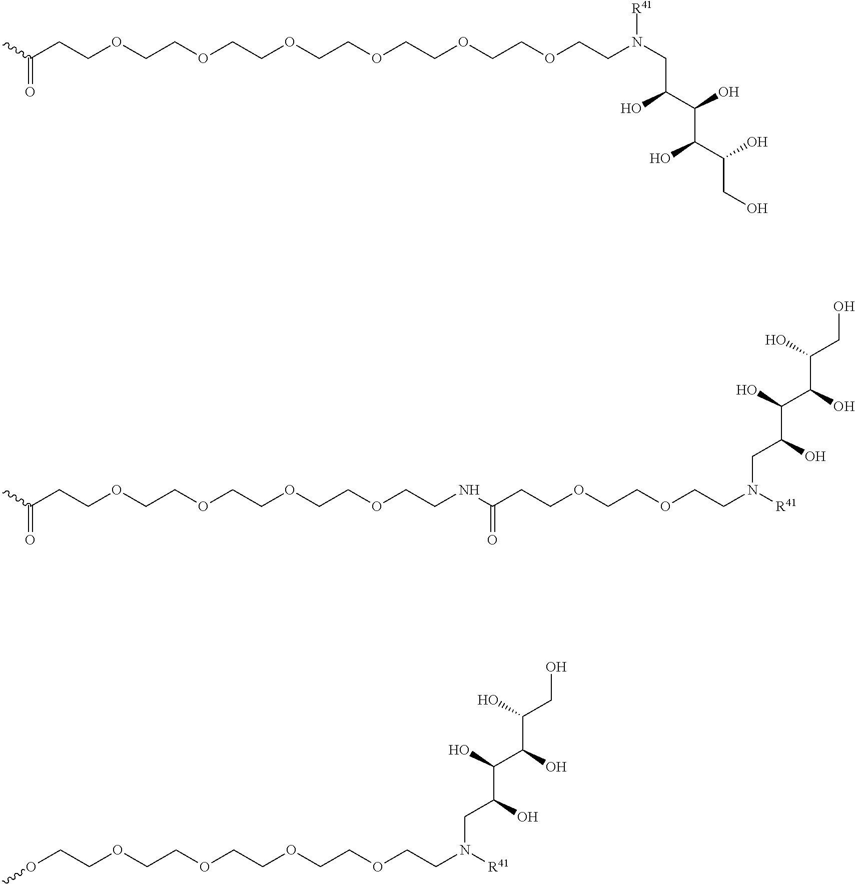
wherein R⁴¹ is a cyclic monosaccharide; and the wavy line is an attachment site to R^{b}, or to the enzyme-cleavable group.

14. The Linker compound of claim 7, wherein R²⁴ and R²⁵ are independently a polyhydroxyl selected from a cyclic monosaccharide, disaccharide, and polysaccharide.

15. The Linker compound of claim 14, comprising a Polar group selected from the following, or a stereoisomer or salt thereof: 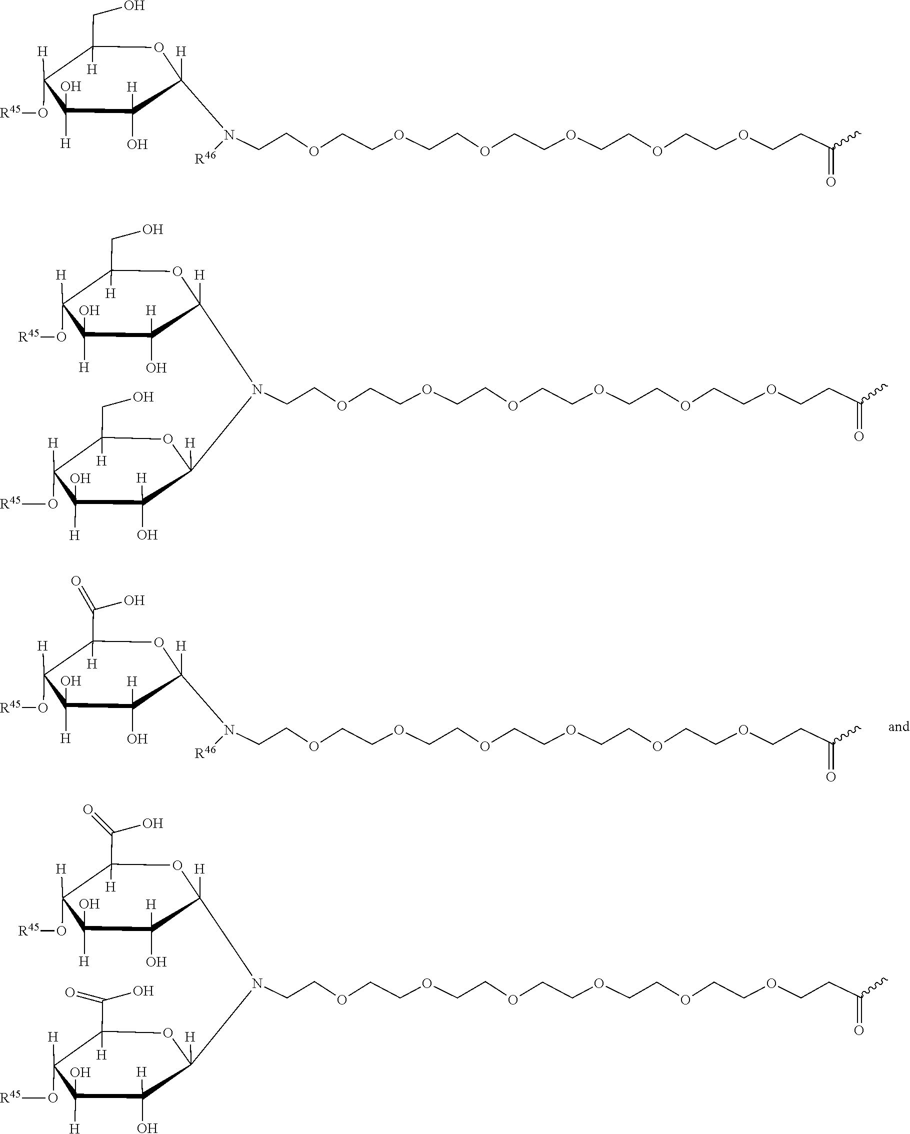
wherein each R⁴⁵ is selected from H and a monosaccharide, a disaccharide, or a polysaccharide; and R⁴⁶ is selected from a cyclic monosaccharide, disaccharide, or polysaccharide; and the wavy line is an attachment site to R^{b}, or to the enzyme-cleavable group.

16. The Linker compound of claim 7, wherein R²⁴ and R²⁵ are independently selected from a linear monosaccharide and a substituted linear monosaccharide, wherein the substituted linear monosaccharide is substituted with a monosaccharide, a disaccharide, or a polysaccharide.

17. The Linker compound of claim 16, comprising a Polar group selected from the following, or a stereoisomer or salt thereof: 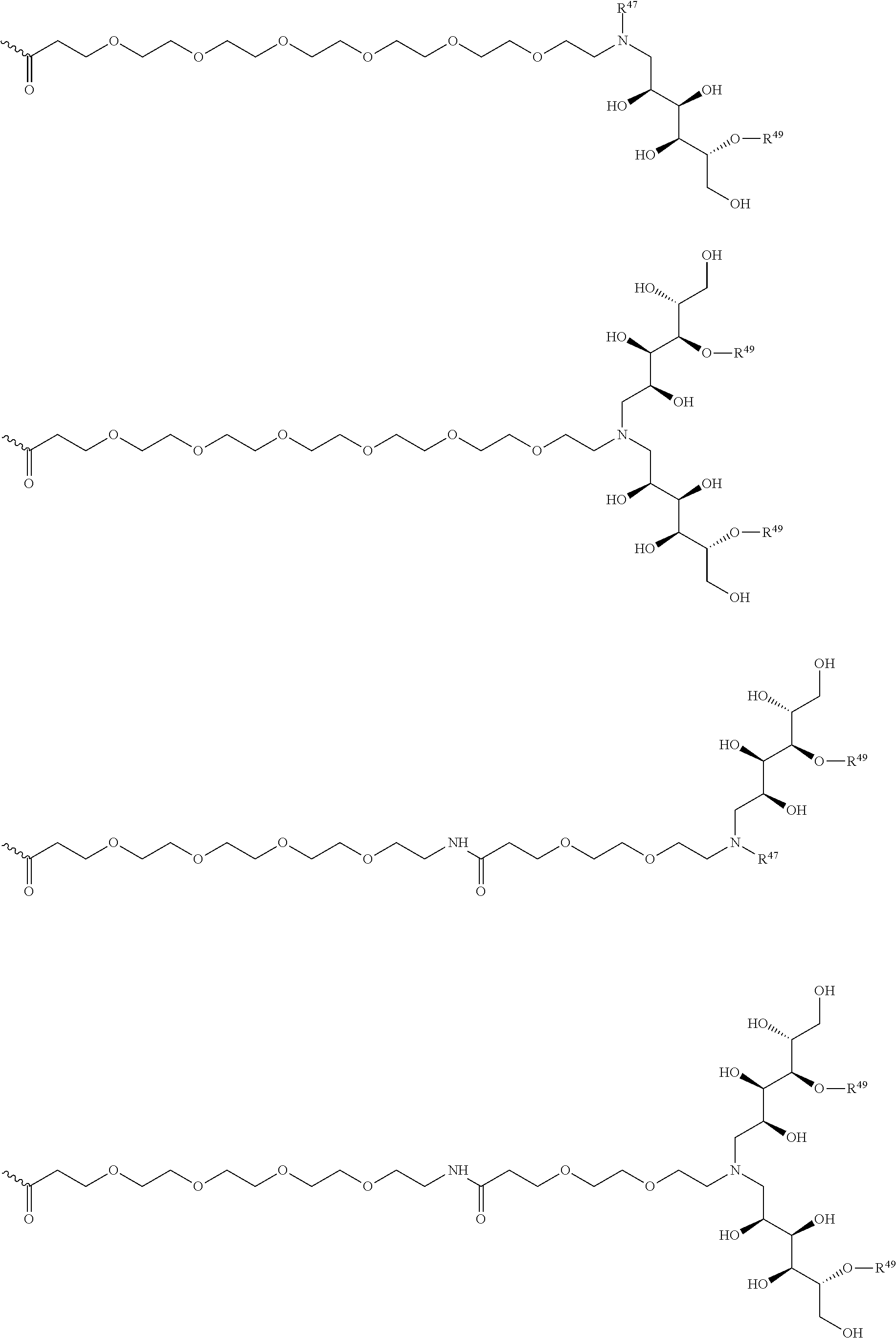
wherein R⁴⁷ is a linear monosaccharide; and each R⁴⁹ is selected from a monosaccharide, a disaccharide, and a polysaccharide; and the wavy line is an attachment site to R^{b}, or to the enzyme-cleavable group.

18. The Linker compound of claim 7, wherein R²⁴ and R²⁵ are independently selected from a linear monosaccharide and a substituted monosaccharide, wherein the substituted linear monosaccharide is substituted with one or more substituents selected from carboxyl, ester, and amide, and optionally further substituted with a monosaccharide, disaccharide, or a polysaccharide.

19. The Linker compound of claim 18, comprising a Polar group selected from the following, or a stereoisomer or salt thereof: 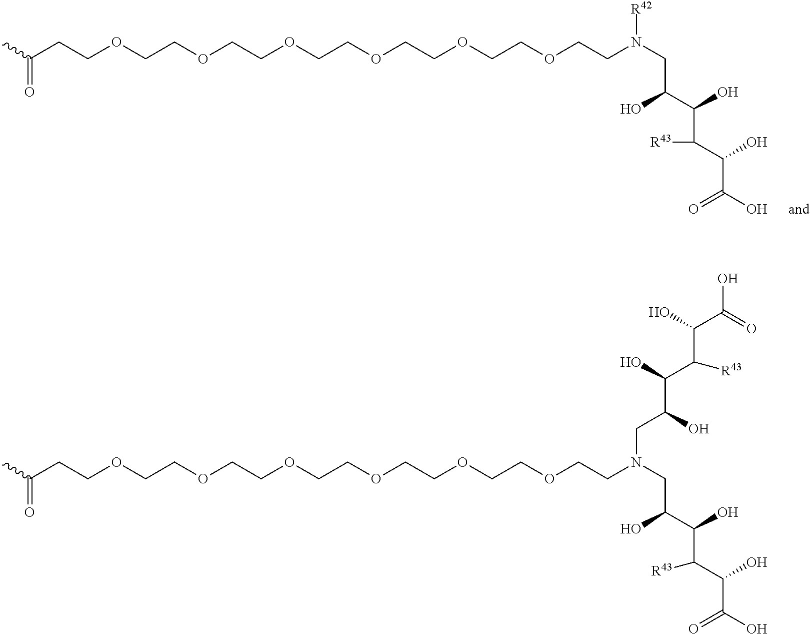
wherein each R⁴² is independently selected from a linear monosaccharide and a substituted linear monosaccharide; each R⁴³ is independently selected from hydroxyl, carboxyl, ester, and amide; and the wavy line is an attachment site to R^{b}, or to the enzyme-cleavable group.

20. The Linker compound of claim 7, wherein one of R²⁴ and R²⁵ is a —C(O)-polyhydroxyl group or substituted —C(O)-polyhydroxyl group, and the other of R²⁴ and R²⁵ is a H, —C(O)-polyhydroxyl group, substituted —C(O)-polyhydroxyl group, polyhydroxyl group or substituted polyhydroxyl group; wherein the substituted —C(O)-polyhydroxyl group and polyhydroxyl group are substituted with a monosaccharide, a disaccharide, a polysaccharide, carboxyl, ester, or amide.

21. The Linker compound of claim 18 or 20, comprising a Polar group selected from the following, or a stereoisomer or salt thereof: 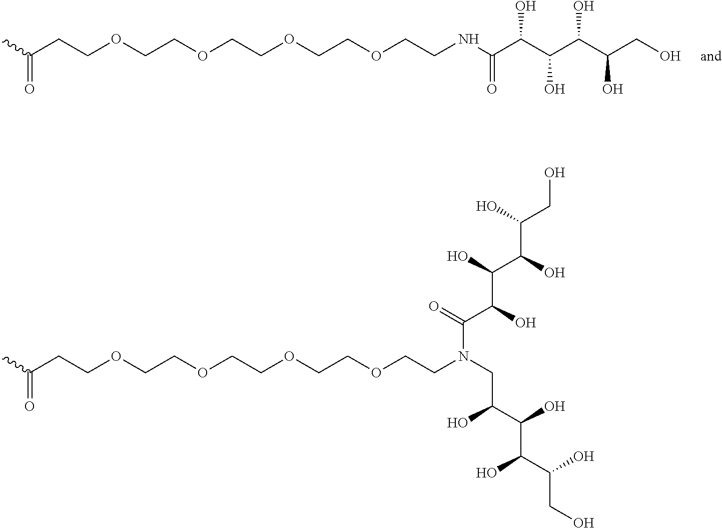
wherein the wavy line is an attachment site to R^{b}, or to the enzyme-cleavable group.

22. The Linker compound of claim 7, wherein R²⁴ and R²⁵ are independently H or substituted —C₁-C₈ alkyl, provided that both R²⁴ and R²⁵ are not H; wherein substituted —C₁-C₈ alkyl is substituted with hydroxyl and/or carboxyl.

23. The Linker compound of claim 22, comprising a Polar group selected from the following, or a stereoisomer or salt thereof: 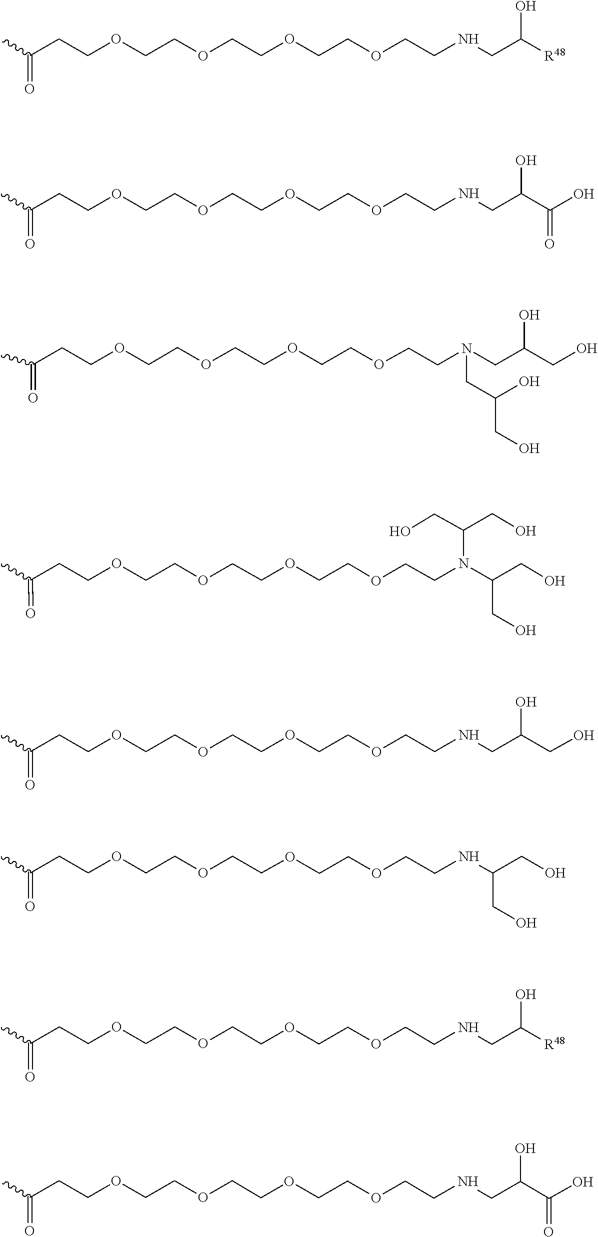 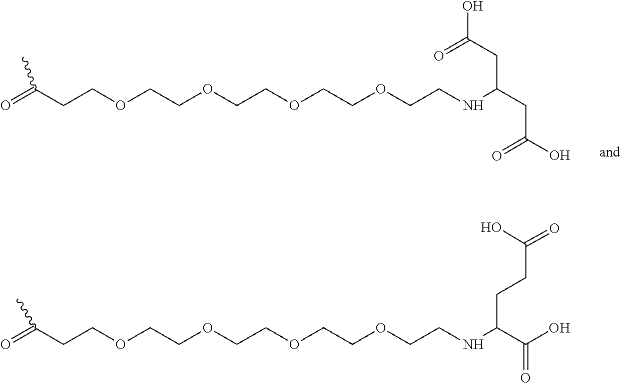
wherein R⁴⁸ is selected from H, OH, CH₂OH, COOH, or —C₁-C₆ alkyl substituted with hydroxyl or carboxyl; and the wavy line is an attachment site to R^{b}, or to the enzyme-cleavable group.

24. The Linker compound of claim 7, wherein one of R²⁴ and R²⁵ is H or substituted —C(O)—C₁-C₈ alkyl, and the other of R²⁴ and R²⁵ is substituted —C(O)—C₁-C₈ alkyl, or substituted —C₁-C₈ alkyl, wherein substituted —C(O)—C₁-C₈ alkyl and substituted —C₁-C₈ alkyl, are substituted with hydroxyl and/or carboxyl.

25. The Linker compound of claim 24, comprising a Polar group selected from the following, or a stereoisomer or salt thereof: 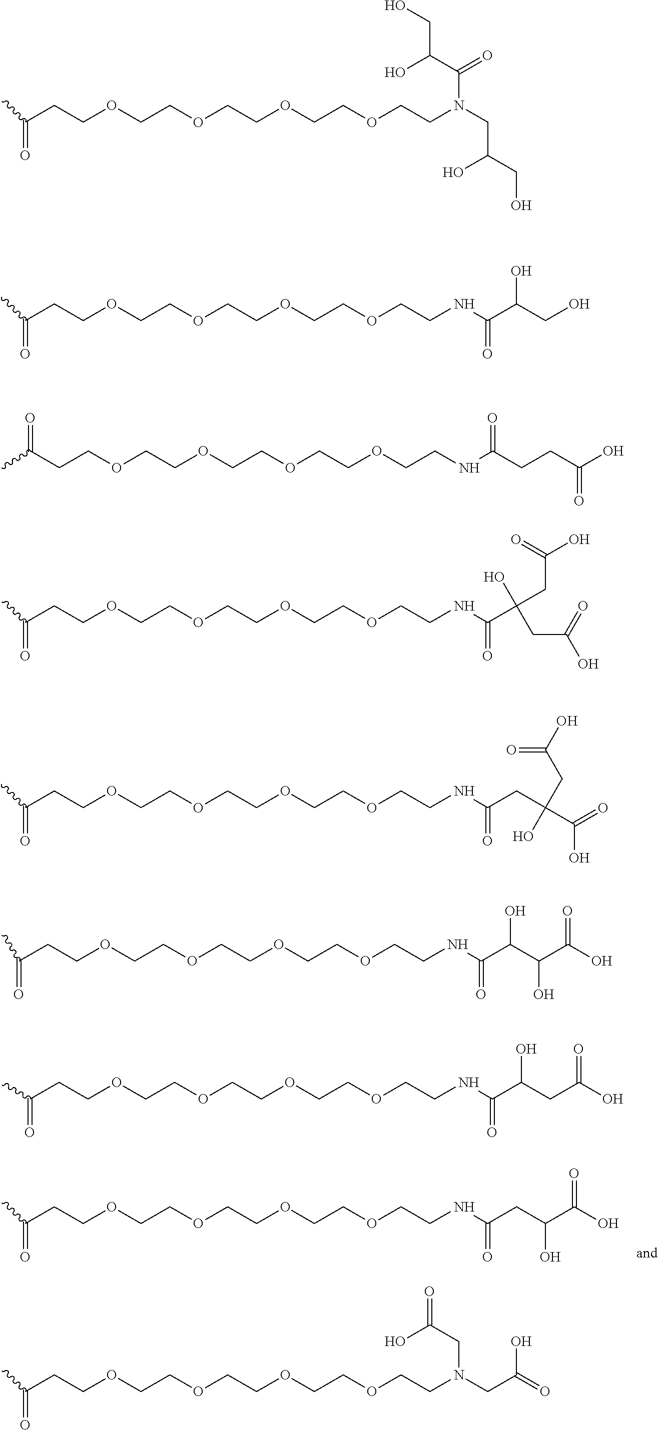
wherein the wavy line is an attachment site to R^{b}, or to the enzyme-cleavable group.

26. The Linker compound of claim 7, wherein R²⁴ and R²⁵ are independently selected from H and a chelator, wherein the chelator is optionally attached to the nitrogen of —NR²⁴R²⁵ by an alkylene, arylene, carbocyclo, heteroarylene, or heterocarbocylo; provided that both R²⁴ and R²⁵ are not H.

27. The Linker compound of claim 26, wherein the chelator is selected from ethylenediaminetetraacetic acid (EDTA), diethylenetriaminepentaacetic acid (DTPA), triethylenetetraminehexaacetic acid (TTHA), benzyl-DTPA, 1,4,7,10-tetraazacyclododecane-N,N′,N″,N′″-tetraacetic acid (DOTA), benzyl-DOTA, 1,4,7-triazacyclononane-N,N′,N″-triacetic acid (NOTA), benzyl-NOTA, 1,4,8,11-tetraazacyclotetradecane-1,4,8,11-tetraacetic acid (TETA) and N,N′-dialkyl substituted piperazine.

28. The Linker compound of claim 27, comprising a Polar group selected from the following, or a stereoisomer or salt thereof: 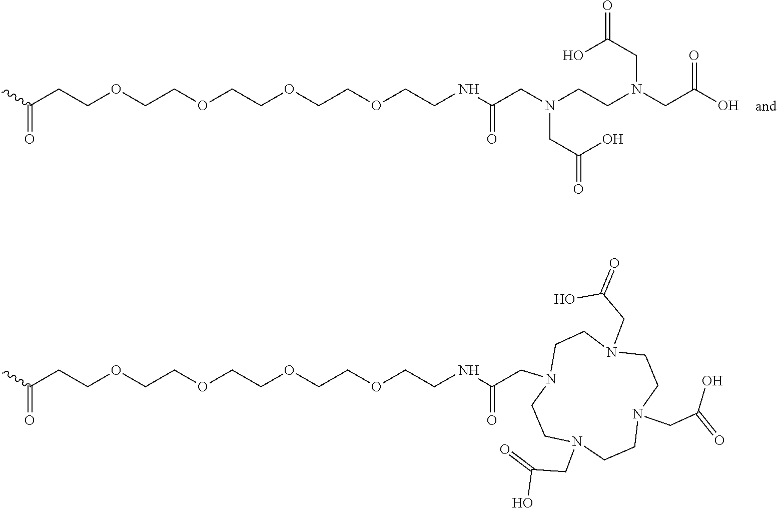
wherein the wavy line is an attachment site to R^{b}, or to the enzyme-cleavable group.

29. The Linker compound of any one of claims 6 to 20, wherein each monosaccharide is independently selected from:
a C5 or C6 sugar selected from glucose, ribose, galactose, mannose, arabinose, 2-deoxyglucose, glyceraldehyde, erythrose, threose, xylose, lyxose, allose, altrose, gulose, idose talose, aldose, and ketose;
a sugar acid selected from gluconic acid, aldonic acid, uronic acid and ulosonic acid; or
an amino sugar selected from glucosamine, N-acetyl glucosamine, galactosamine, and N-acetyl galactosamine.

30. The Linker compound of any one of claims 1 to 29, wherein the attachment site is formed from a functional group of a precursor compound of the Polar group, said functional group selected from halo, aldehyde, carboxyl, amino, alkynyl, azido, hydroxyl, carbonyl, carbamate, thiol, urea, thiocarbamate, thiourea, sulfonamide, acyl sulfonamide, alkyl sulfonate, triazole, azadibenzocyclooctyne, hydrazine, carbonylalkylheteroaryl, and protected forms thereof.

31. The Linker compound of any one of claims 1 to 6, comprising a Polar group having a formula selected from the following: 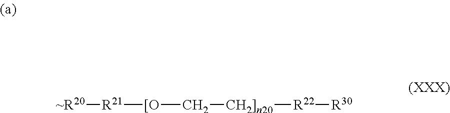
or a stereoisomer or salt thereof, wherein:
R²⁰ is an attachment group to site R^{b}, or to the enzyme-cleavable group;
R²¹ and R²² are each independently, a bond or C₁-C₃ alkylene groups;
R³⁰ is selected from an optionally substituted C₃-C₁₀ carbocycle; thiourea; optionally substituted thiourea; urea; optionally substituted urea; sulfamide; alkyl sulfamide; acyl sulfamide, optionally substituted alkyl sulfamide; optionally substituted acyl sulfamide; sulfonamide; optionally substituted sulfonamide; guanidine, including alkyl and aryl guanidine; phosphoramide; or optionally substituted phosphoramide; or R³⁰ is selected from azido, alkynyl, substituted alkynyl, —NH—C(O)-alkynyl, —NH—C(O)— alkynyl-R⁶⁵; cyclooctyne; —NH-cyclooctyne, —NH—C(O)-cyclooctyne, or —NH-(cyclooctyne)₂; wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and
n20 is 2 to 26; 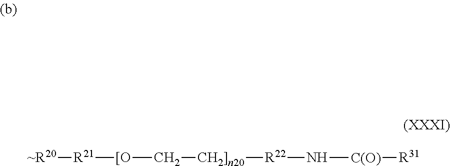
or a stereoisomer or salt thereof, wherein:
R²⁰ is an attachment group to site R^{b}, or to the enzyme-cleavable group;
R²¹ and R²² are each, independently, a bond or C₁-C₃ alkylene groups;
R³¹ is a branched polyethylene glycol chain, each branch having 1 to 26 ethylene glycol subunits and each branch having an R³⁵ at its terminus;
R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and
n20 is 2 to 26; 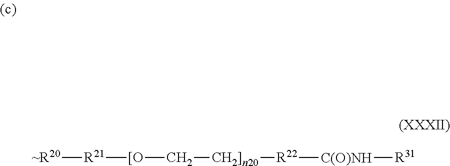
or a stereoisomer or salt thereof, wherein:
R²⁰ is an attachment group to site R^{b}, or to the enzyme-cleavable group;
R²¹ and R²² are each, independently, a bond or C₁-C₃ alkylene groups;
R³¹ is a branched polyethylene glycol chain, each branch, independently, having 1 to 26 ethylene glycol subunits and each branch having an R³⁵ at its terminus;
R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle and optionally substituted heteroaryl; and
n20 is 2 to 26; 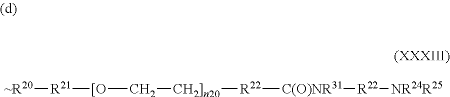
or a stereoisomer or salt thereof, wherein:
R²⁰ is an attachment group to site R^{b}, or to the enzyme-cleavable group;
R³¹ is H or R²²—NR²⁴R²⁵; R²¹ and R²² are each, independently, a bond or C₁-C₃ alkylene groups;
R²⁴ and R²⁵ are each independently selected from a H; polyhydroxyl group; substituted polyhydroxyl group; —C(O)-polyhydroxyl group; substituted —C(O)-polyhydroxyl group, provided that R²⁴ and R²⁵ are not both H; and
n20 is 2 to 26; 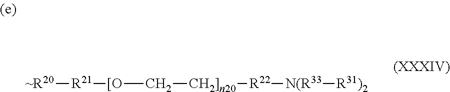
or a stereoisomer or salt thereof, wherein:
R²⁰ is an attachment group to site R^{b}, or to the enzyme-cleavable group;
R²¹ and R²² are each, independently, a bond or C₁-C₃ alkylene groups;
R³¹ is a branched polyethylene glycol chain, each branch having 1 to 26 ethylene glycol subunits and each branch having an R³⁵ at its terminus;
R³³ is C₁-C₃ alkylene, C₁-C₃ alkylene-C(O), —C(O)—C₁-C₃ alkylene, or —C(O)—C₁-C₃ alkylene-C(O);
R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and
n20 is 2 to 26; 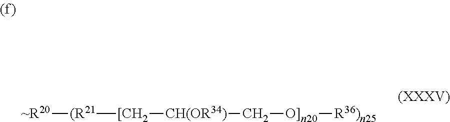
or a stereoisomer or salt thereof, wherein:
R²⁰ is an attachment group to site R^{b}, or to the enzyme-cleavable group;
each R²¹ is independently a bond, —O— or C₁-C₃ alkylene group;
each R³⁴ is independently H, —[CH₂—CH(OH)—CH₂—O]ₙ₂₀—R³⁶, —C(O)—NR²⁴R²⁵ or —C(O)N(R^{N})—C₁-C₆alkylene-NR²⁴R²⁵;
R^{N} is H or C₁-C₄alkyl;
R²⁴ and R²⁵ are each independently selected from a H; polyhydroxyl group; or
substituted polyhydroxyl group, provided that both R²⁴ and R²⁵ are not H;
each R³⁶ is independently H, C₁-C₆alkylene-C(OH)H—NR⁴⁴R⁴⁵, C₁-C₆alkylene-C(OH)H—C₁-C₆alkylene-NR⁴⁴R⁴⁵, —C(O)—NR²⁴R²⁵, —C(O)N(R^{N})—C₁-C₆alkylene-NR²⁴R²⁵, C₁-C₆alkylene-C(O)NR²⁴R²⁵ or C₁-C₆alkylene-CO₂R³⁷;
each R³⁷ is independently H or C₁-C₆ alkyl;
R⁴⁴ and R⁴⁵ are each independently selected from a H; polyhydroxyl group; substituted polyhydroxyl group; —C(O)-polyhydroxyl group; and substituted —C(O)-polyhydroxyl group, provided that both R⁴⁴ and R⁴⁵ are not H;
each n20 is independently 1 to 26; and
n25 is 1 or 2; 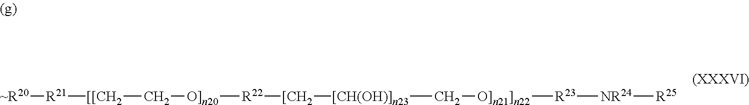
or a stereoisomer or salt thereof, wherein:
R²⁰ is an attachment group to site R^{b}, or to the enzyme-cleavable group;
R²¹, R²² and R²³ are each independently a bond or C₁-C₃ alkylene group;
R²⁴ and R²⁵ are each independently selected from a H; polyhydroxyl group; substituted polyhydroxyl group; —C(O)-polyhydroxyl group; and substituted —C(O)-polyhydroxyl group, provided that R²⁴ and R²⁵ are not both H;
each n20 is independently 0 to 26, and each n21 is independently 0 to 26, with the proviso that at least one of n20 or n21 is 2 to 26;
n22 is 1 to 5;
each n23 is independently 1 or 2; 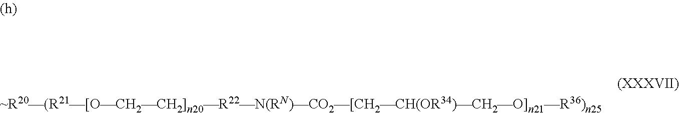
or a stereoisomer or salt thereof, wherein:
R²⁰ is an attachment group to site R^{b}, or to the enzyme-cleavable group;
R²¹ and R²² are each independently a bond or C₁-C₃ alkylene groups;
R^{N} is H or C₁-C₄alkyl;
R²⁴ and R²⁵ are each independently selected from a H; polyhydroxyl group; and
substituted polyhydroxyl group, provided that both R²⁴ and R²⁵ are not H;
each R³⁴ is independently H, —[CH₂—CH(OH)—CH₂—O]ₙ₂₀—R³⁶ or —C(O)N(R^{N})—C₁-C₆alkylene-NR²⁴R²⁵;
each R³⁶ is independently H, C₁-C₆alkylene-C(OH)H—NR⁴⁴R⁴⁵, C₁-C₆alkylene-C(OH)H—C₁-C₆alkylene-NR⁴⁴R⁴⁵, —C(O)N(R^{N})—C₁-C₆alkylene-NR²⁴R²⁵, C₁-C₆alkylene-C(O)NR²⁴R²⁵ or C₁-C₆alkylene-CO₂R³⁷;
each R³⁷ is independently H or C₁-C₆ alkyl;
R⁴⁴ and R⁴⁵ are each independently selected from a H; polyhydroxyl group; substituted polyhydroxyl group; —C(O)-polyhydroxyl group; and substituted —C(O)-polyhydroxyl group; provided that both R⁴⁴ and R⁴⁵ are not H;
n20 is 2 to 26;
n21 is 1 to 26; and
n25 is 1 or 2; 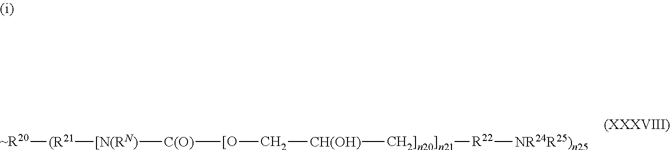
or a stereoisomer or salt thereof, wherein:
R²⁰ is an attachment group to site R^{b}, or to the enzyme-cleavable group;
R²¹ and R²² are each independently a bond or C₁-C₃ alkylene groups;
R^{N} is H or C₁-C₄alkyl;
R²⁴ and R²⁵ are each independently selected from a H; polyhydroxyl group; and substituted polyhydroxyl group, provided that R²⁴ and R²⁵ are not both H;
n20 is 2 to 26;
n21 is 1 to 4; and
n25 is 1, 2 or 3; 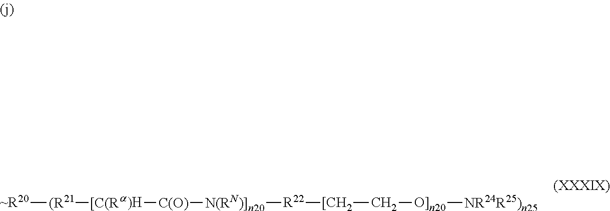
or a stereoisomer or salt thereof, wherein:
R²⁰ is an attachment group to site R^{b}, or to the enzyme-cleavable group;
R²¹ and R²² are each, independently, a bond, C₁-C₃ alkylene, —C₁-C₃alkylene-[O—CH₂—CH₂-]ₙ₂₀, —[CH₂—CH₂—O]ₙ₂₀—C₁-C₃alkylene- or —C₁-C₃alkylene-[O—CH₂—CH₂-]ₙ₂₀-C(O)—;
each R^{α} is independently H or —R²²—NR²⁴R²⁵;
each R^{N} is independently H, C₁-C₆ alkyl or —R²²—NR²⁴R²⁵;
R²⁴ and R²⁵ are each independently selected from a H; polyhydroxyl group; substituted polyhydroxyl group; —C(O)-polyhydroxyl group; substituted —C(O)-polyhydroxyl group;
substituted —C(O)—C₁-C₈ alkyl; a chelator; —C(O)—R²⁸, wherein R²⁸ is a Sugar unit of formula (XII) or (XIII), provided that R²⁴ and R²⁵ are not both H;
each n20 is independently 0 to 26, with the proviso that at least one n20 is 2 to 26; and
n25 is 1 or 2; or 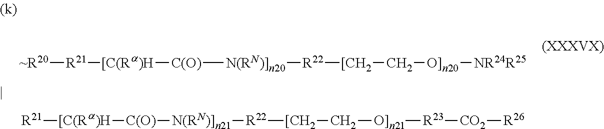
or a stereoisomer or salt thereof, wherein:
R²⁰ is an attachment group to site R^{b}, or to the enzyme-cleavable group;
R²¹, R²² and R²³ are each, independently, a bond, C₁-C₃ alkylene, —C₁-C₃alkylene-[O—CH₂—CH₂-]ₙ₂₀, —[CH₂—CH₂—O]ₙ₂₀—C₁-C₃alkylene- or —C₁-C₃alkylene-[O—CH₂—CH₂-]ₙ₂₀-C(O)—;
each R^{α} is independently H or —R²²—NR²⁴R²⁵;
each R^{N} is independently H, C₁-C₆ alkyl or —R²²—NR²⁴R²⁵;
R²⁴ and R²⁵ are each independently selected from a H; polyhydroxyl group; substituted polyhydroxyl group; —C(O)-polyhydroxyl group; substituted —C(O)-polyhydroxyl group;
substituted —C(O)—C₁-C₈ alkyl; a chelator; and —C(O)—R²⁸, where R²⁸ is a Sugar unit of formula (XII) or (XIII), provided that R²⁴ and R²⁵ are not both H;
R²⁶ is H or C₁-C₆ alkyl;
each n20 is independently 0 to 26, with the proviso that at least one n20 is 2 to 26; and
each n21 is independently 0 to 26, with the proviso that at least one n21 is 2 to 26.

32. The Linker compound of any one of claims 1 to 6, comprising a Polar group having a formula selected from the following, or a stereoisomer or salt thereof: 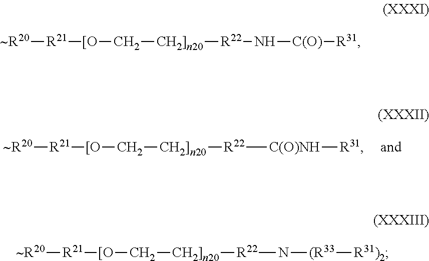
wherein:
R²⁰ is an attachment group to site R^{b}, or to the enzyme-cleavable group;
R²¹ and R²² are each, independently, a bond or C₁-C₃ alkylene groups;
R³¹ is a branched polyethylene glycol chain, each branch having 1 to 26 ethylene glycol subunits and each branch having an R³⁵ at its terminus;
R³³ is C₁-C₃ alkylene, —C₁-C₃ alkylene-C(O), —C(O)—C₁-C₃ alkylene or —C(O)—C₁-C₃ alkylene-C(O);
R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; the wavy (˜) line indicates an attachment site to R²⁰; and n20 is 2 to 26.

33. The Linker compound of claim 31 or 32, comprising a Polar group formed from a precursor group selected from the following: 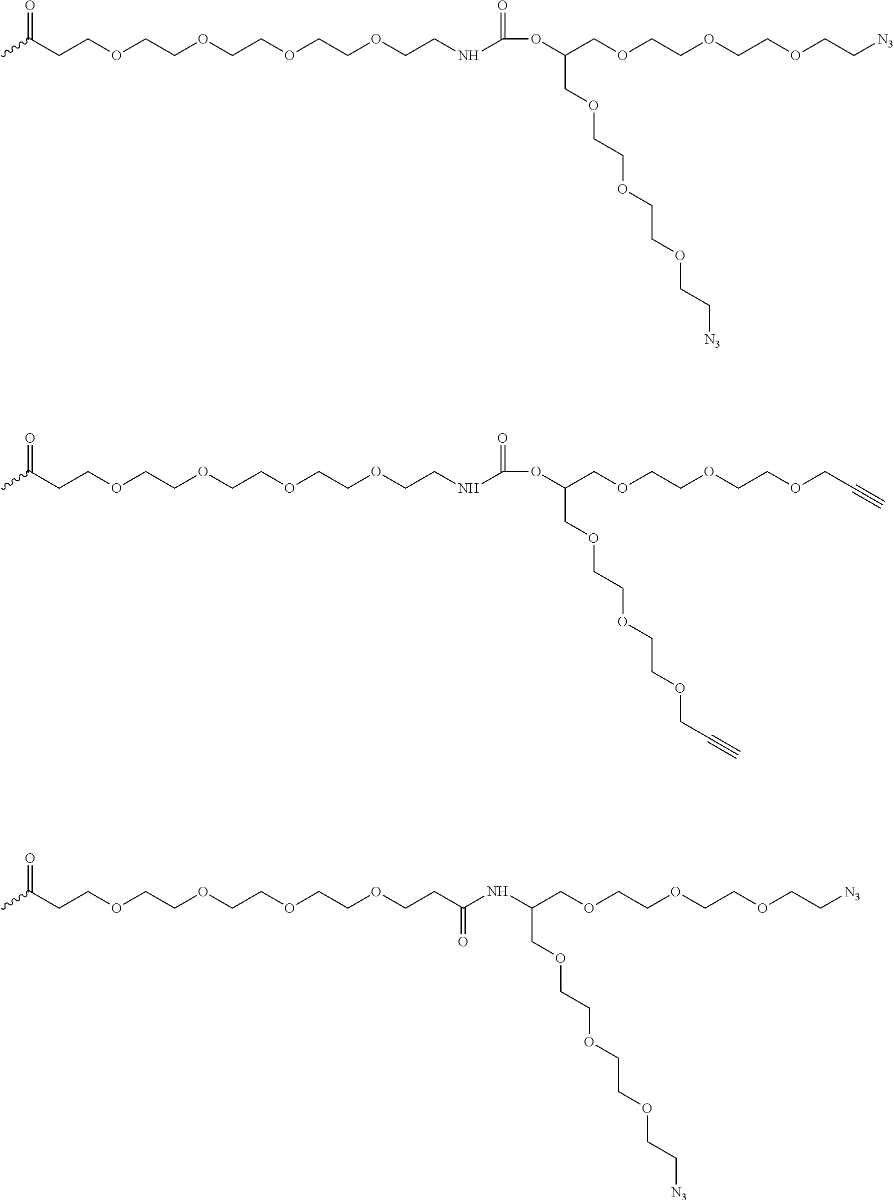 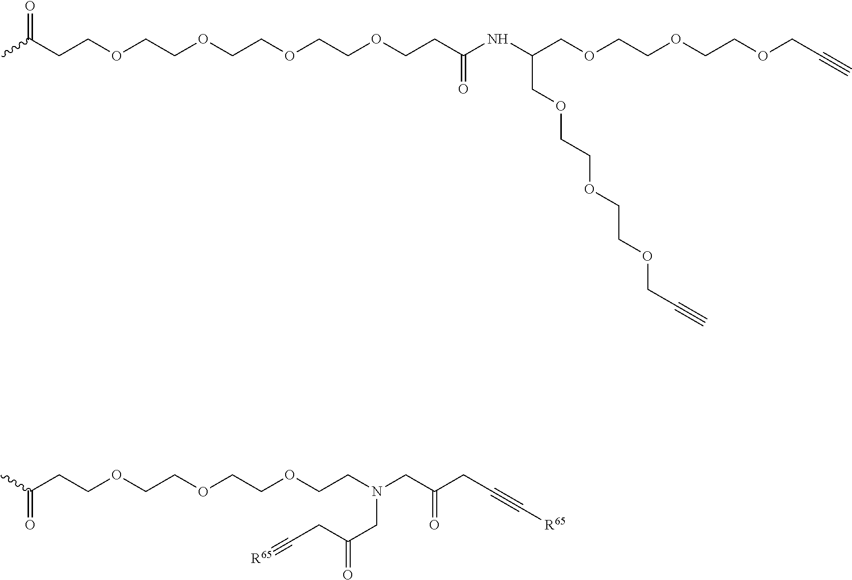
wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and the wavy line is an attachment site to R^{b}, or to the enzyme-cleavable group.

34. The Linker compound of any one of claims 31 to 33, wherein the attachment site to R^{b}, or to the enzyme-cleavable group is formed from a functional group of a precursor compound of the Polar group, said functional group selected from halo, aldehyde, carboxyl, amino, alkynyl, azido, hydroxyl, carbonyl, carbamate, thiol, urea, thiocarbamate, thiourea, sulfonamide, acyl sulfonamide, alkyl sulfonate, triazole, azadibenzocyclooctyne, hydrazine, carbonylalkylheteroaryl, and protected forms thereof.

35. The Linker compound of any one of claims 1-6, comprising a Polar group having a formula:
˜R²⁰—(R⁴³—R⁴¹—[O—CH₂—CH₂]ₙ₄₀—R⁴²—R⁴³—(NR⁴⁴R⁴⁵)ₙ₄₁)n₄₂ (XL)
or a stereoisomer or salt thereof, wherein:
R²⁰ is an attachment group to site R^{b}, or to the enzyme-cleavable group;
R⁴¹ and R⁴² are each, independently, a bond or C₁-C₆ alkylene;
each R⁴³ is, independently, selected from a bond, C₁-C₁₂ alkylene, —OC₁-C₁₂ alkylene, —C(═O)—, —NR^{a}—C₁-C₁₂ alkylene, —C₁-C₁₂ alkylene-NR^{a}—, —C(O)—C₁-C₁₂ alkylene, —C₁-C₁₂ alkylene-C(O)—, —NR^{a}—C₁-C₁₂ alkylene-C(O)—, —C(O)—C₁-C₁₂ alkylene-NR^{a}—, —NR^{a}—C(O)—NR^{a}—, —NR^{a}—C(O)—, —NR^{a}—C(O)—C₁-C₁₂ alkylene, —C(O)—NR^{a}—C₁-C₁₂ alkylene, -heteroarylene, heteroaryl-C₁-C₁₂ alkylene, heteroaryl-C₁-C₁₂ alkylene-C(O)—, or —C(O)NR⁴⁶R⁴⁷, wherein each alkylene is optionally substituted with hydroxyl, SO₃H and/or oxo, R^{a} is H, C₁-C₆ alkyl, a polyhydroxyl group, or a substituted polyhydroxyl group, and one of R⁴⁶ and R⁴⁷ is H or C₁-C₁₂ alkylene and the other is C₁-C₁₂ alkylene, wherein one of the C₁-C₂ alkylenes is bound to NR⁴⁴R⁴⁵ at the nitrogen atom;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate; n40 is 2 to 26, provided that R⁴⁴ and R⁴⁵ are not both H;
n40 is 2 to 26;
n41 is 1 to 6; and
n42 is 1 to 6.

36. The Linker compound of any one of claims 1-6, comprising a Polar group having a formula: 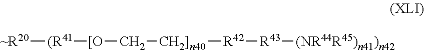
or a stereoisomer or salt thereof, wherein:
R²⁰ is an attachment group to site R^{b}, or to the enzyme-cleavable group;
R⁴¹ and R⁴² are each, independently, a bond or C₁-C₆ alkylene;
R⁴³ is selected from a bond, C₁-C₁₂ alkylene, —OC₁-C₁₂ alkylene, —C(═O)—, —NR^{a}—C₁-C₁₂ alkylene, —C₁-C₁₂ alkylene-NR^{a}—, —C(O)—C₁-C₁₂ alkylene, —C₁-C₁₂ alkylene-C(O)—, —NR^{a}—C₁-C₁₂ alkylene-C(O)—, —C(O)—C₁-C₁₂ alkylene-NR^{a}—, —NR^{a}—C(O)—NR^{a}—, —NR^{a}—C(O)—, —NR^{a}—C(O)—C₁-C₁₂ alkylene, C(O)—NR^{a}—C₁-C₁₂ alkylene, -heteroarylene, heteroaryl-C₁-C₁₂ alkylene, heteroaryl-C₁-C₁₂ alkylene-C(O)—, and —C(O)NR⁴⁶R⁴⁷, wherein each alkylene is optionally substituted with hydroxyl, SO₃H and/or oxo, R^{a} is H, C₁-C₆ alkyl, a polyhydroxyl group, or a substituted polyhydroxyl group and one of R⁴⁶ and R⁴⁷ is H or C₁-C₁₂ alkylene and the other is C₁-C₁₂ alkylene, wherein one of the C₁-C₂ alkylenes is bound to NR⁴⁴R⁴⁵ at the nitrogen atom;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, provided that R⁴⁴ and R⁴⁵ are not both H;
n40 is 1 to 26;
n41 is 1 to 6; and
n42 is 1 to 6.

37. The Linker compound of any one of claims 1-6, comprising a Polar group having a formula: 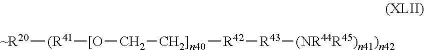
or a stereoisomer or salt thereof, wherein:
R²⁰ is an attachment group to site R^{b}, or to the enzyme-cleavable group;
R⁴¹ and R⁴² are each, independently, a bond or C₁-C₃ alkylene;
R⁴³ is selected from a bond, C₁-C₆ alkylene, —OC₁-C₁₂ alkylene, —C(═O)—, —NR^{a}—C₁-C₁₂ alkylene, —C₁-C₆ alkylene-NR^{a}—, —C(O)—C₁-C₆ alkylene, -C₁-C₈ alkylene-C(O)—, —NR^{a}—C₁-C₆ alkylene-C(O)—, —C(O)—C₁-C₆ alkylene-NR^{a}—, —NR^{a}—C(O)—NR^{a}—, —NR^{a}—C(O)—, —NR^{a}—C(O)—C₁-C₈ alkylene, —C(O)—NR^{a}—C₁-C₁₂ alkylene, -heteroarylene, heteroaryl-C₁-C₆ alkylene, heteroaryl-C₁-C₆ alkylene-C(O)—, and —C(O)NR⁴⁶R⁴⁷, wherein each alkylene is optionally substituted with hydroxyl, SO₃H, and/or oxo, R^{a} is H, C₁-C₆ alkyl, a polyhydroxyl group, or a substituted polyhydroxyl group and one of R⁴⁶ and R⁴⁷ is H or C₁-C₆ alkylene and the other is C₁-C₁₂ alkylene, wherein one of the C₁-C₂ alkylenes is bound to NR⁴⁴R⁴⁵ at the nitrogen atom;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, provided that R⁴⁴ and R⁴⁵ are not both H;
n40 is 1 to 16;
n41 is 1 to 4; and
n42 is 1 to 4.

38. The Linker compound of any one of claims 7, 31, 32, and 35-37, wherein R²⁰ is formed from a functional group of a precursor compound of the Polar group, said functional group selected from halo, aldehyde, carboxyl, amino, alkynyl, azido, hydroxyl, carbonyl, carbamate, thiol, urea, thiocarbamate, thiourea, sulfonamide, acyl sulfonamide, alkyl sulfonate, triazole, azadibenzocyclooctyne, hydrazine, carbonylalkylheteroaryl, or protected forms thereof.

39. The Linker compound of any one of claims 7, 31, 32, and 35-37, wherein R²⁰ comprises one of the following structures: 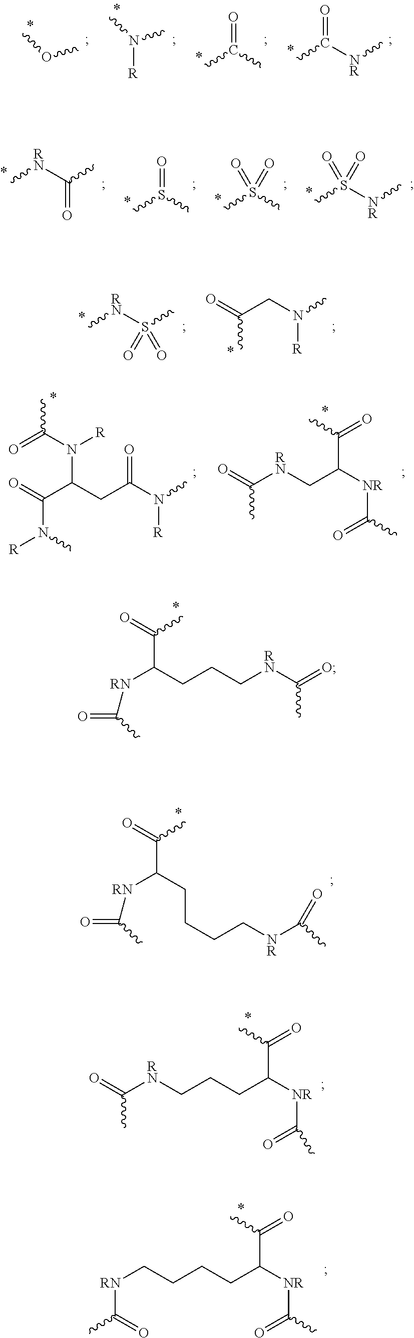 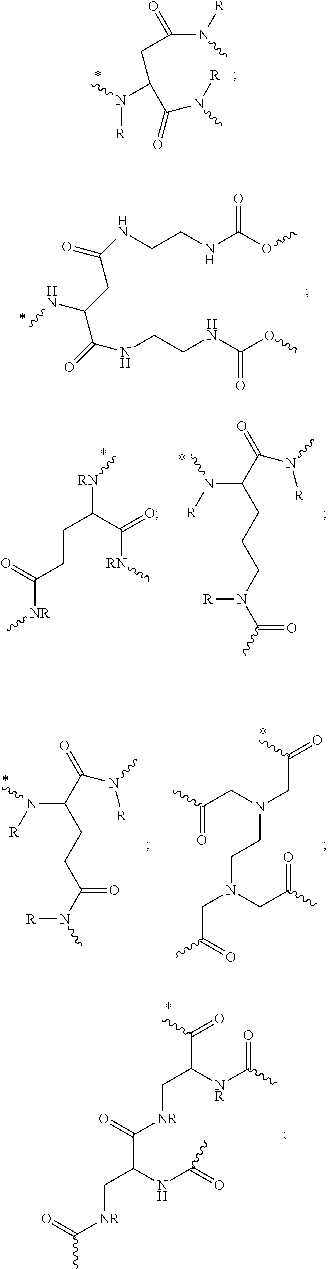 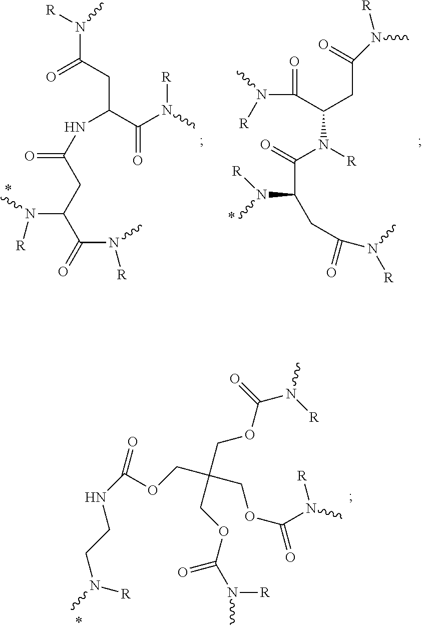 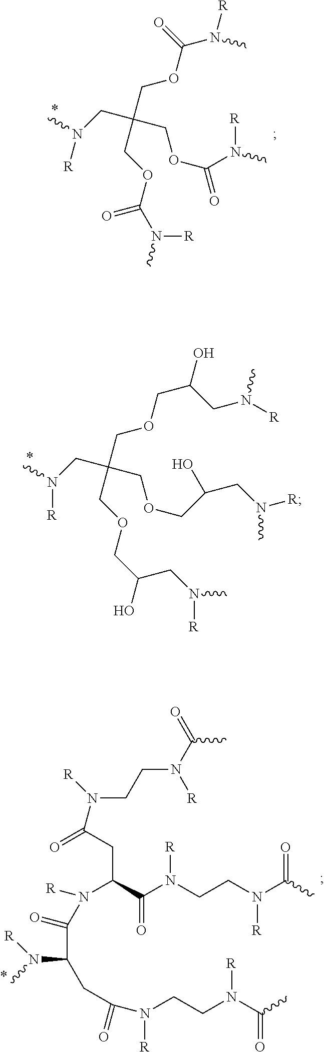 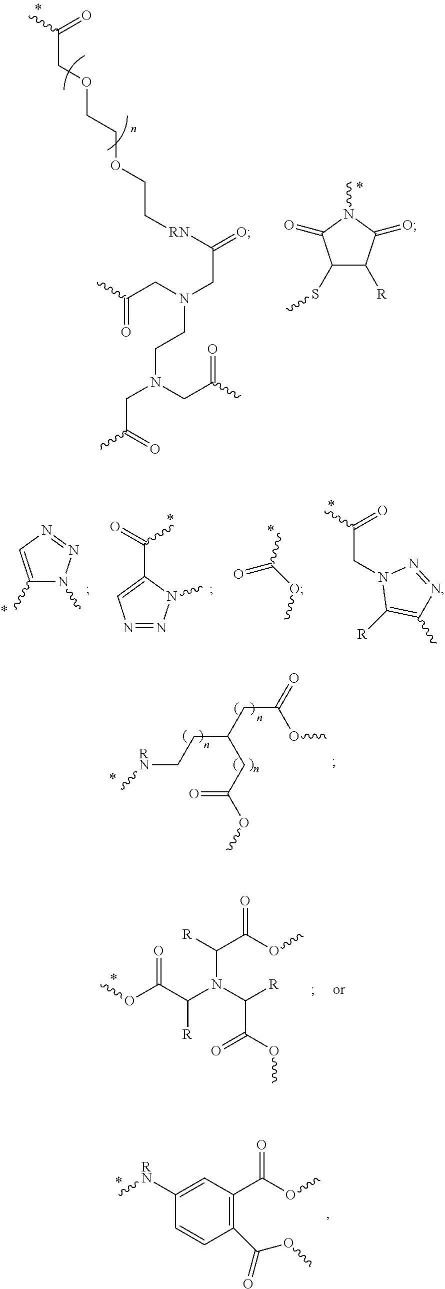
or a stereoisomer thereof, wherein R is H, C₁-C₆ alkyl or polyhydroxyl group, n is 0 to 12, the ( [symbol] , *) indicates an attachment site to R^{b}, or to the enzyme-cleavable group, and the ( [symbol] ) indicates an attachment site to a remainder portion of the Polar group.

40. The compound of any one of claims 7, 31, 32, and 35-37, wherein R²⁰ has one of the following structures: 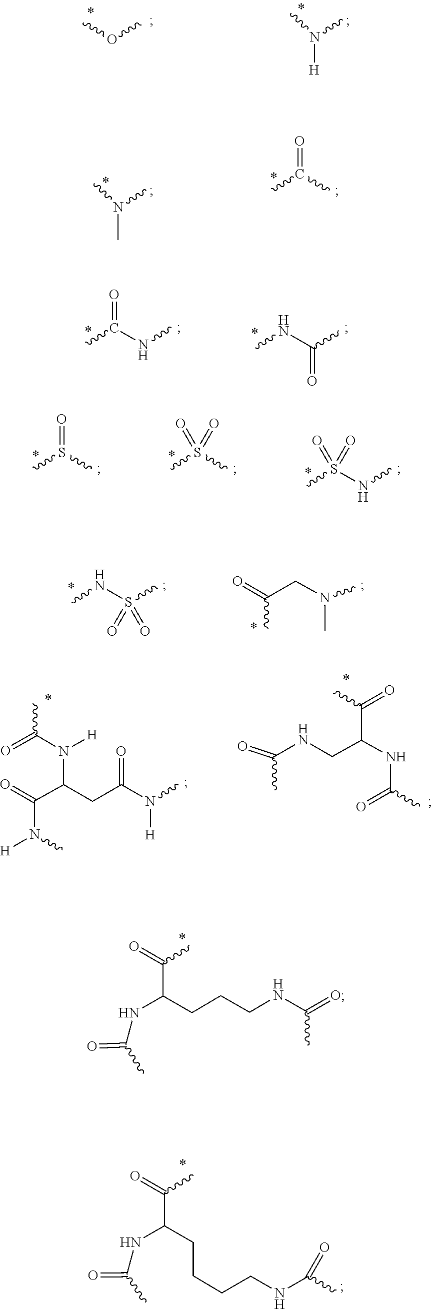 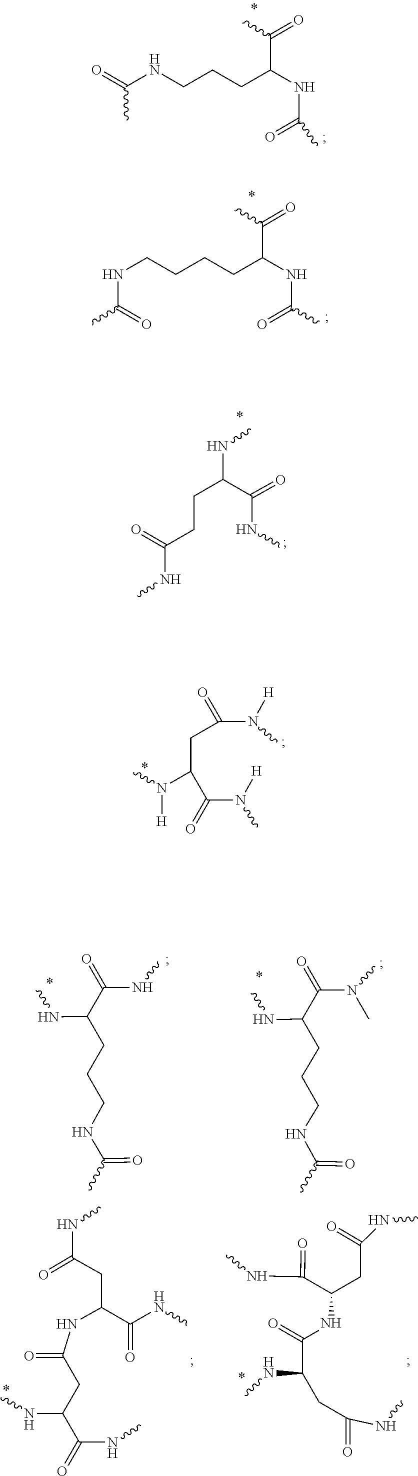 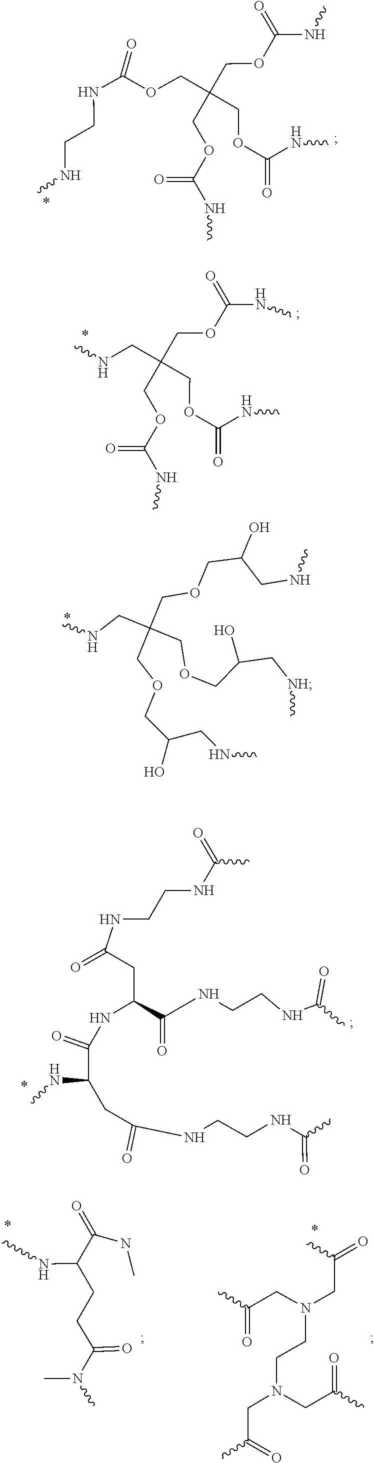 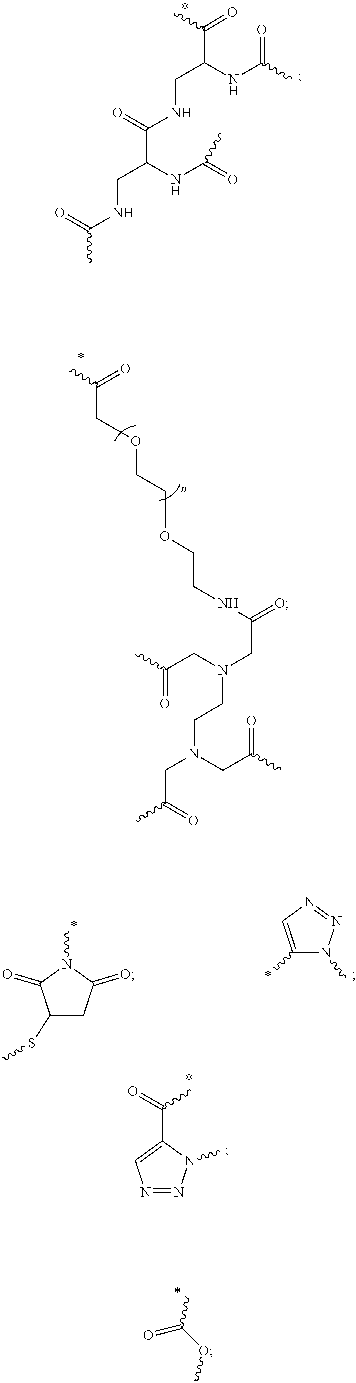 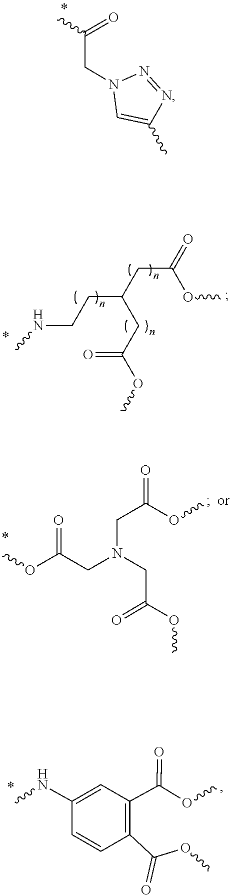
or a stereoisomer thereof, wherein n=0 to 12, the ( [symbol] *) indicates an attachment site to R^{b}, or to the enzyme-cleavable group, and the ( [symbol] ) indicates an attachment site to a remainder portion of the Polar group.

41. The Linker compound of any one of claims 35-40, wherein R⁴³—(NR⁴⁴R⁴⁵)ₙ₄₁ has one of the following structures: 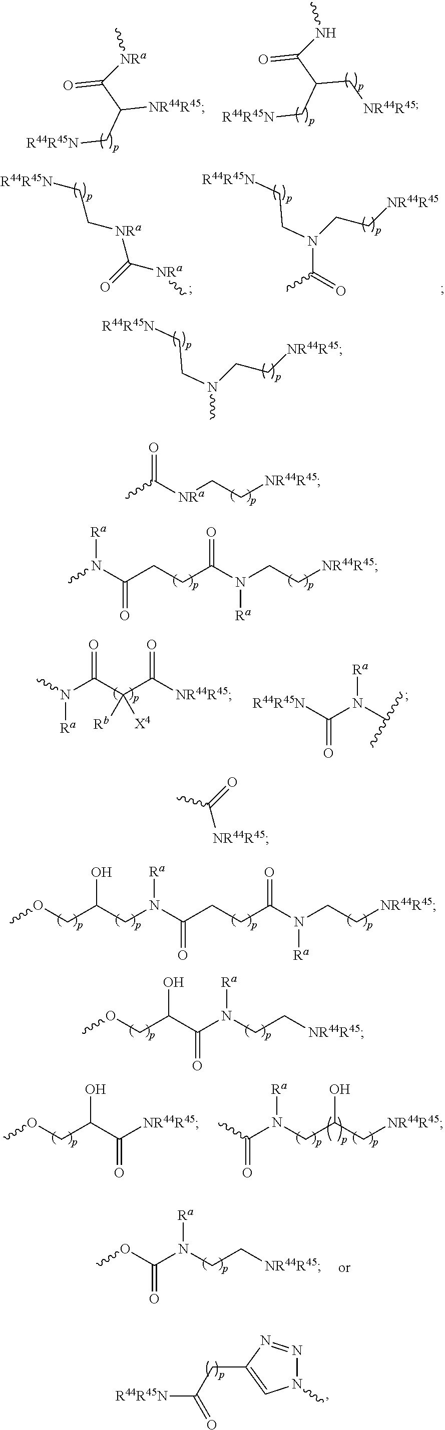
or a stereoisomer thereof, wherein R^{a} is H, C₁-C₈ alkyl, a polyhydroxyl group, or a substituted polyhydroxyl group; p is an integer from 1 to 6, and the ( [symbol] ) indicates the attachment site of R⁴³ to the remainder of the Polar group.

42. The Linker compound of anyone of claims 35-40, wherein R⁴³—(NR⁴⁴R⁴⁵)ₙ₄₁ has one of the following structures: 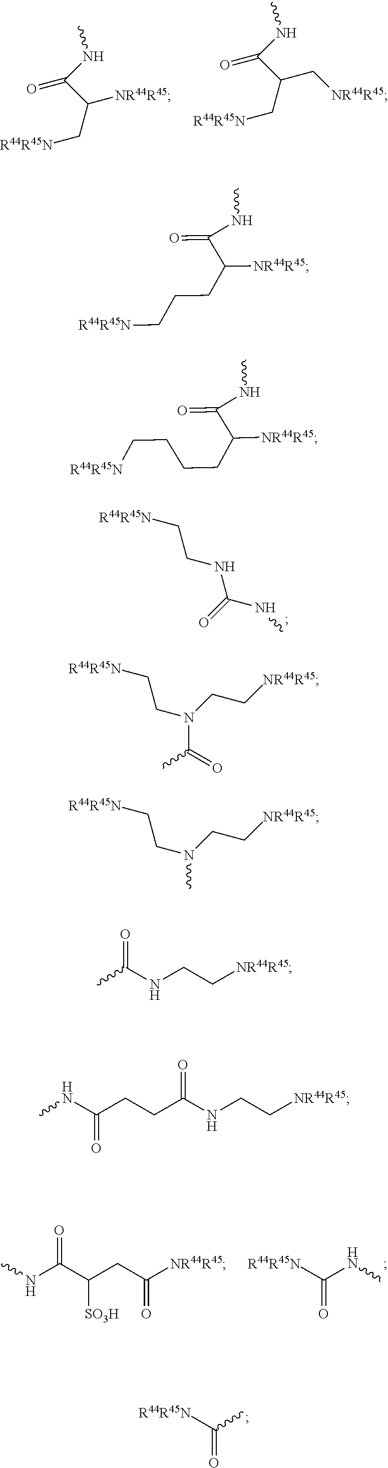 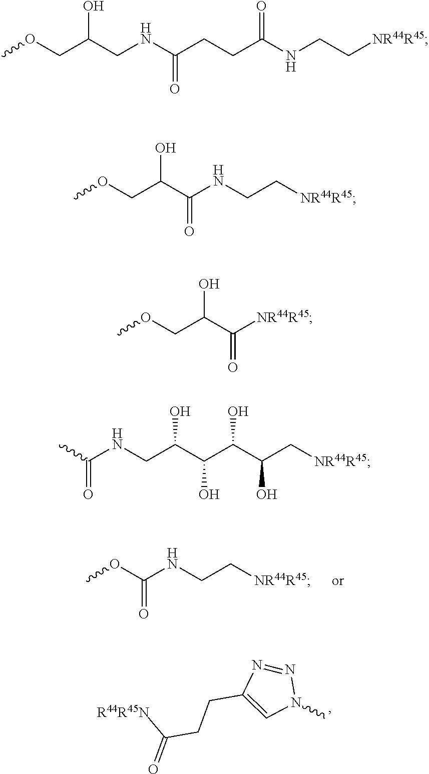
or a stereoisomer thereof, wherein the ( [symbol] ) indicates the attachment site of R⁴³ to the remainder of the Polar group.

43. The Linker compound of any one of claims 35-42, wherein —NR⁴⁴R⁴⁵ has one of the following structures: 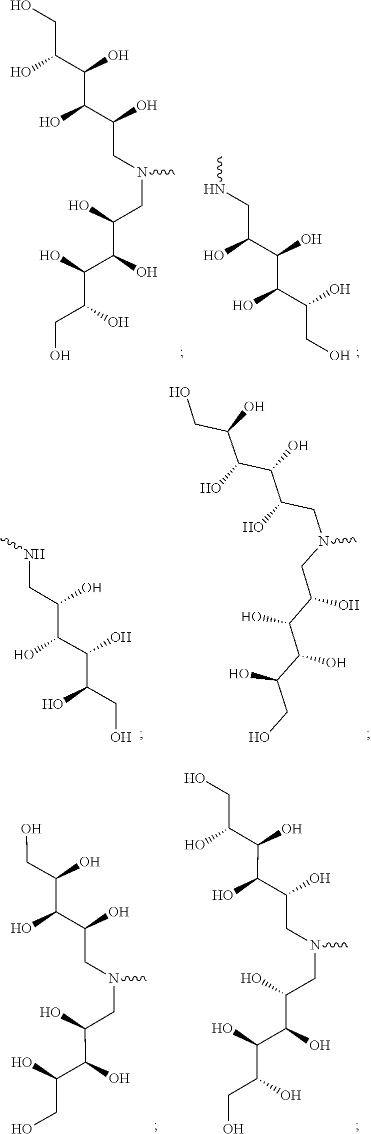 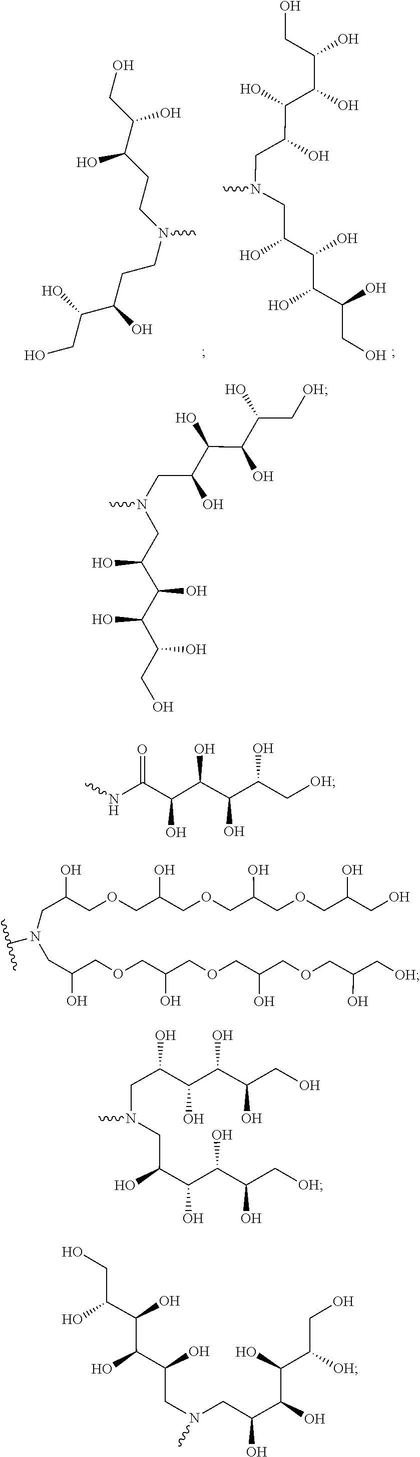 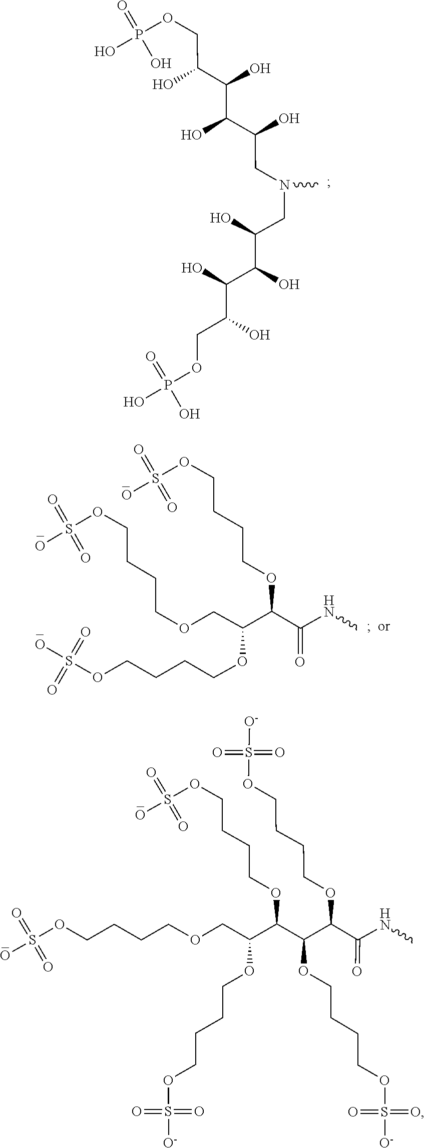
or a stereoisomer thereof, wherein the ( [symbol] ) indicates the attachment site of —NR⁴⁴R⁴⁵ to the remainder of the Polar group.

44. The Linker compound of any one of claims 1-44, comprising a Polar group having one of the following structures prior to attachment to the Linker Unit: 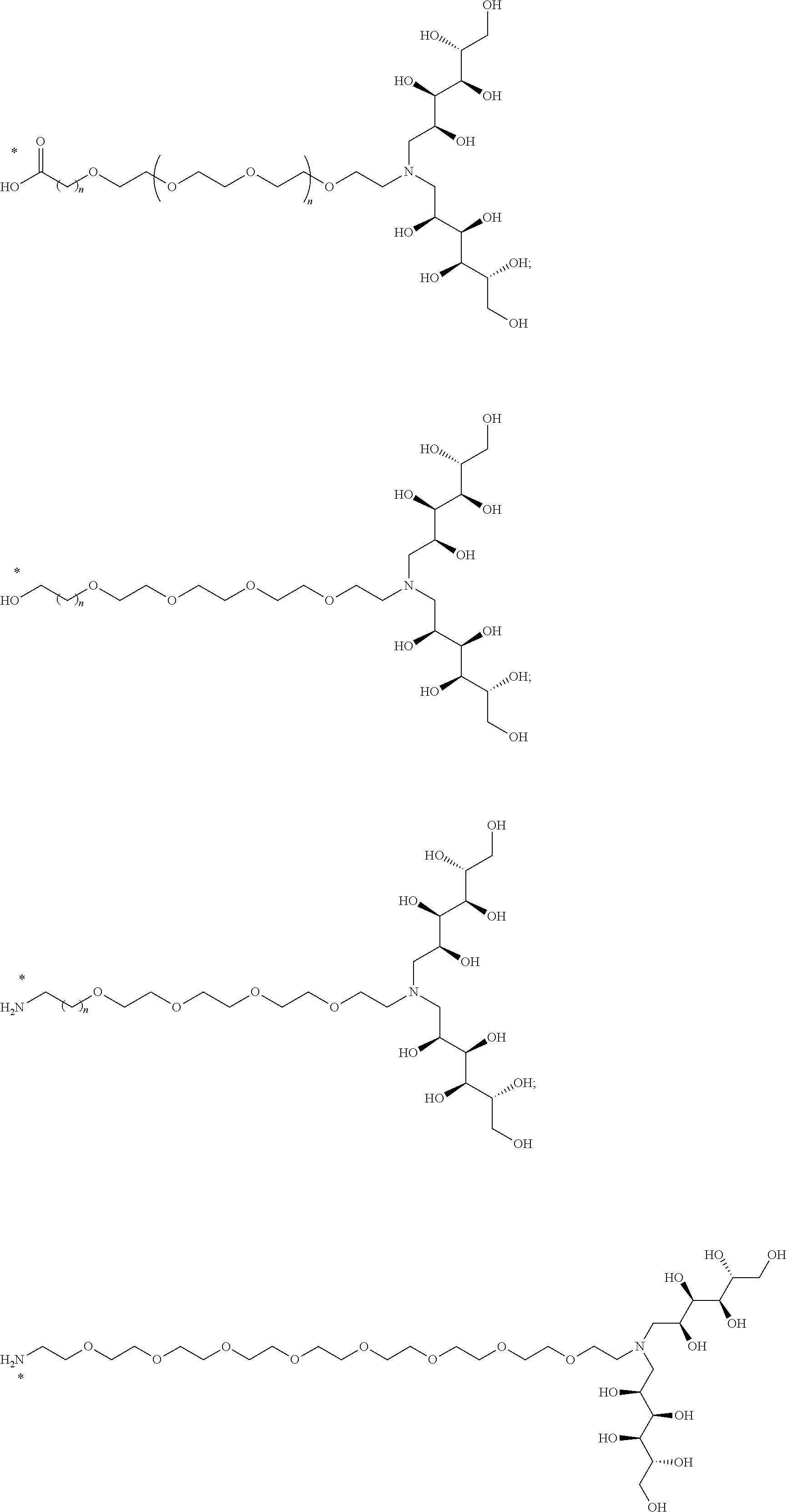 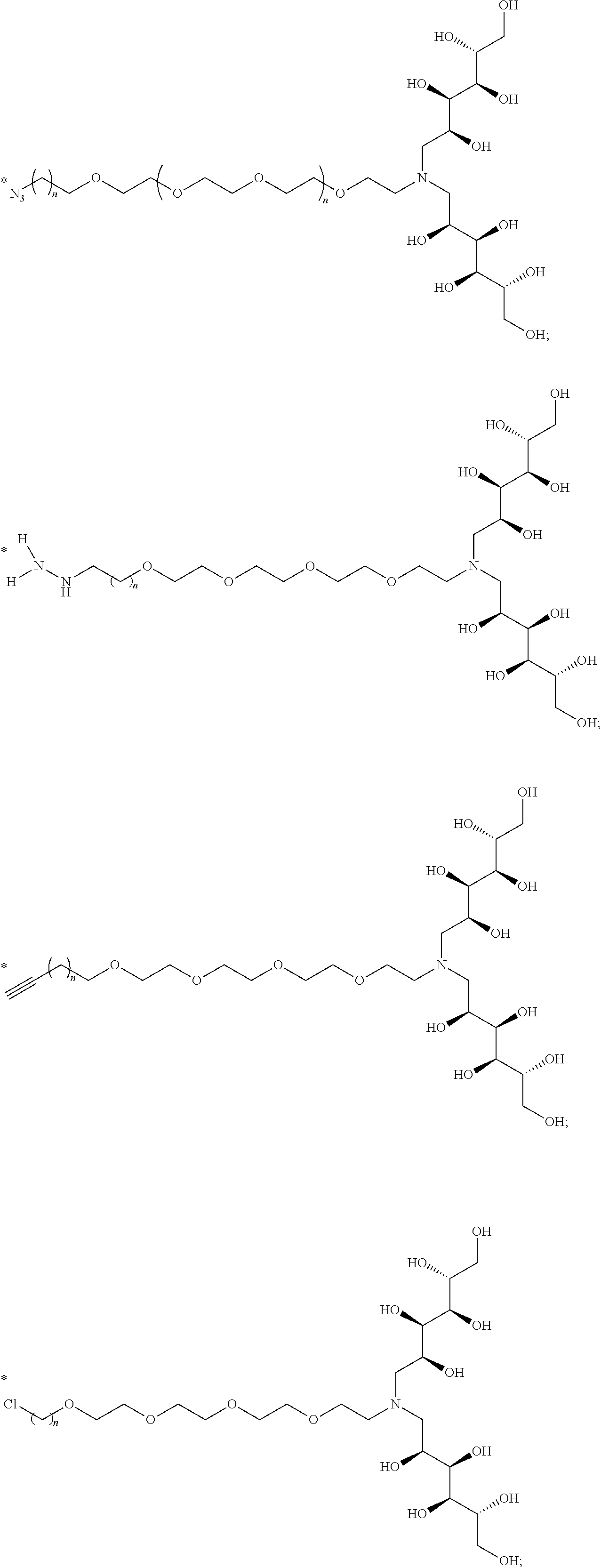 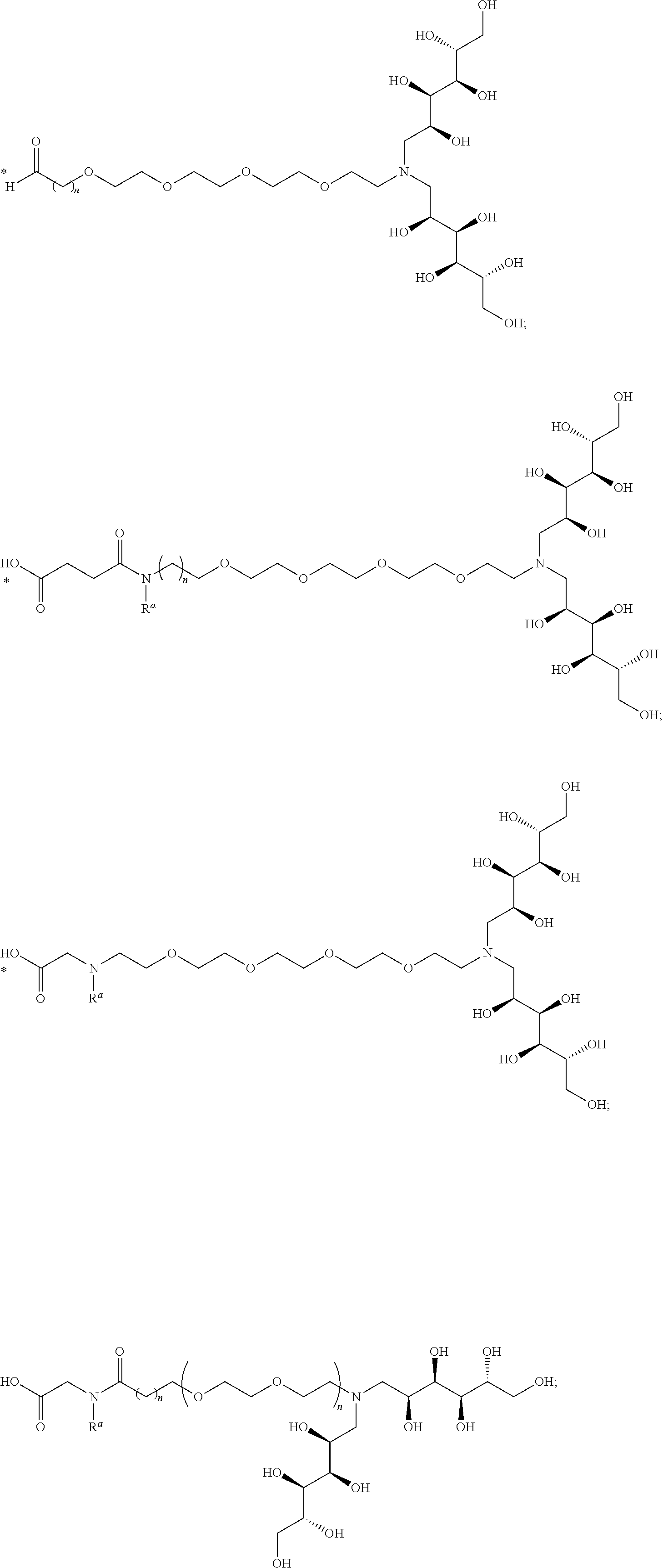 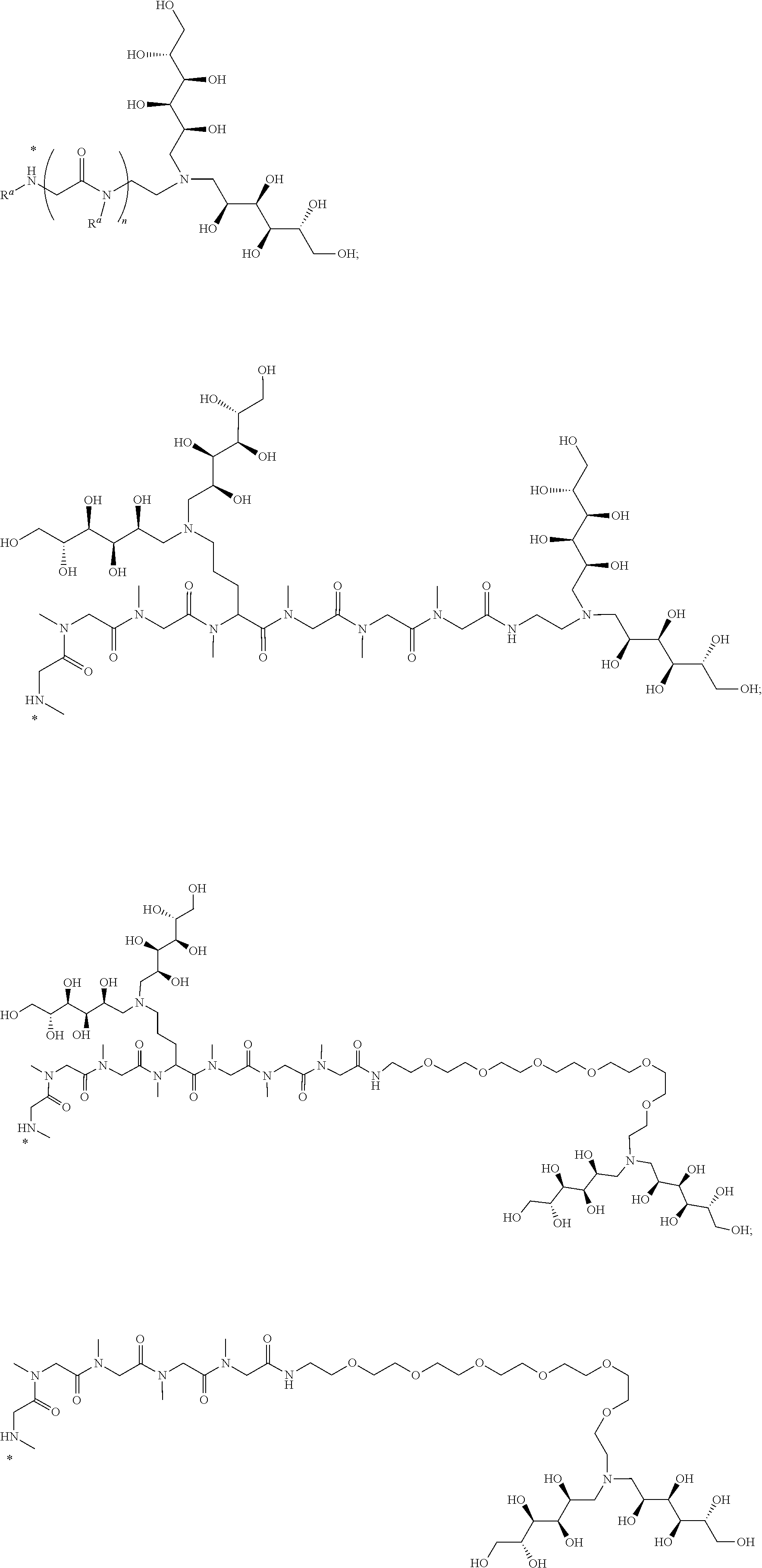 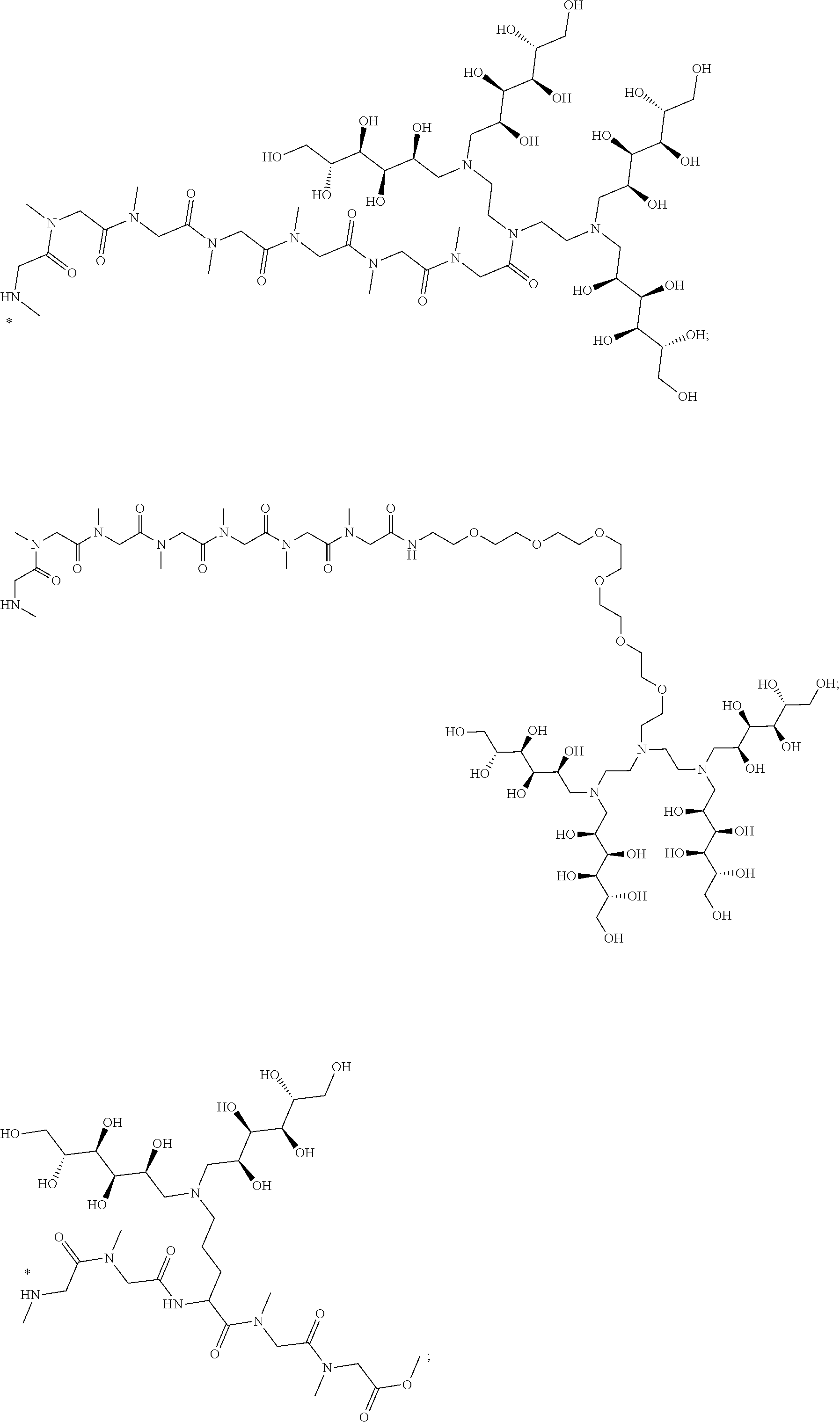 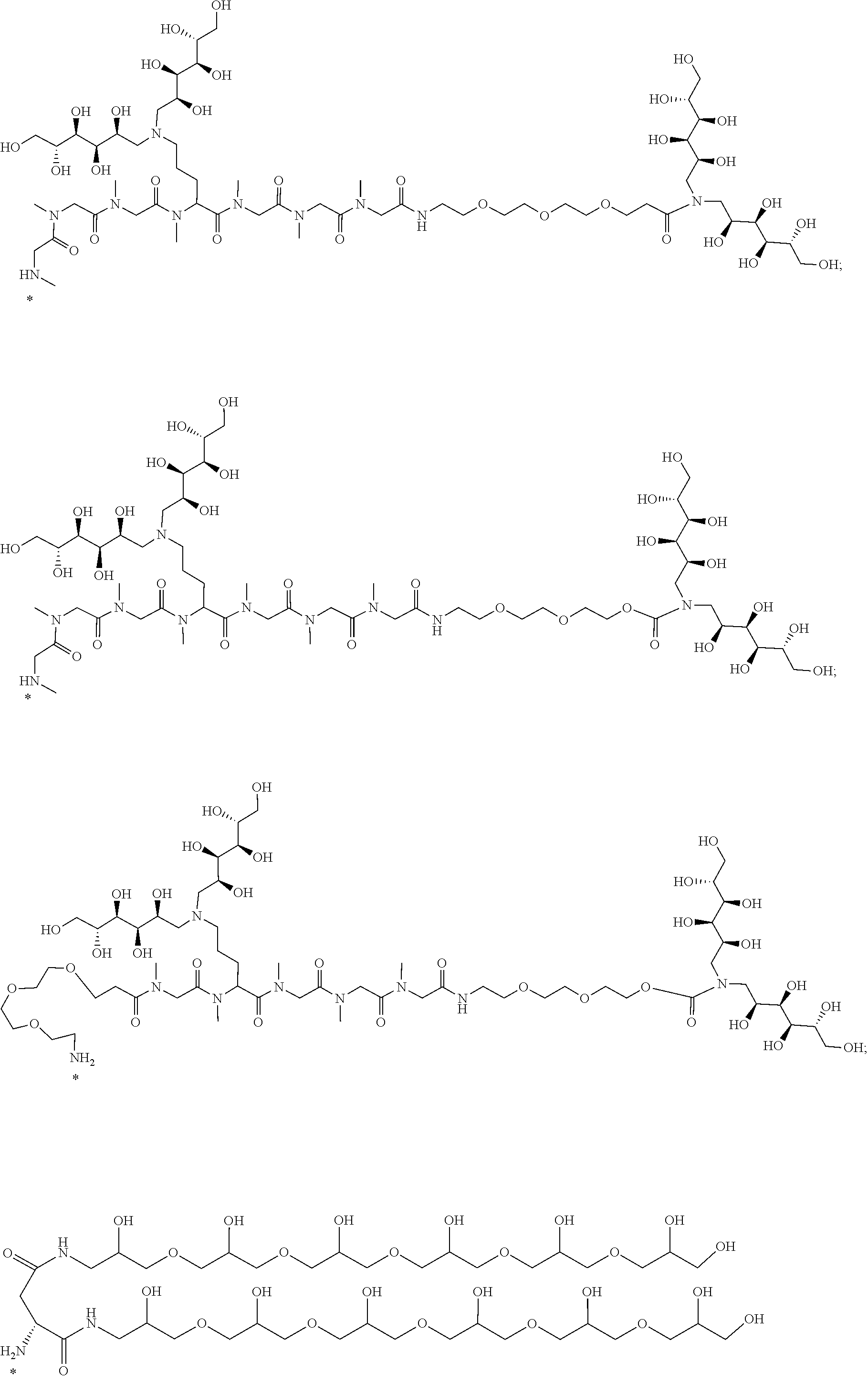 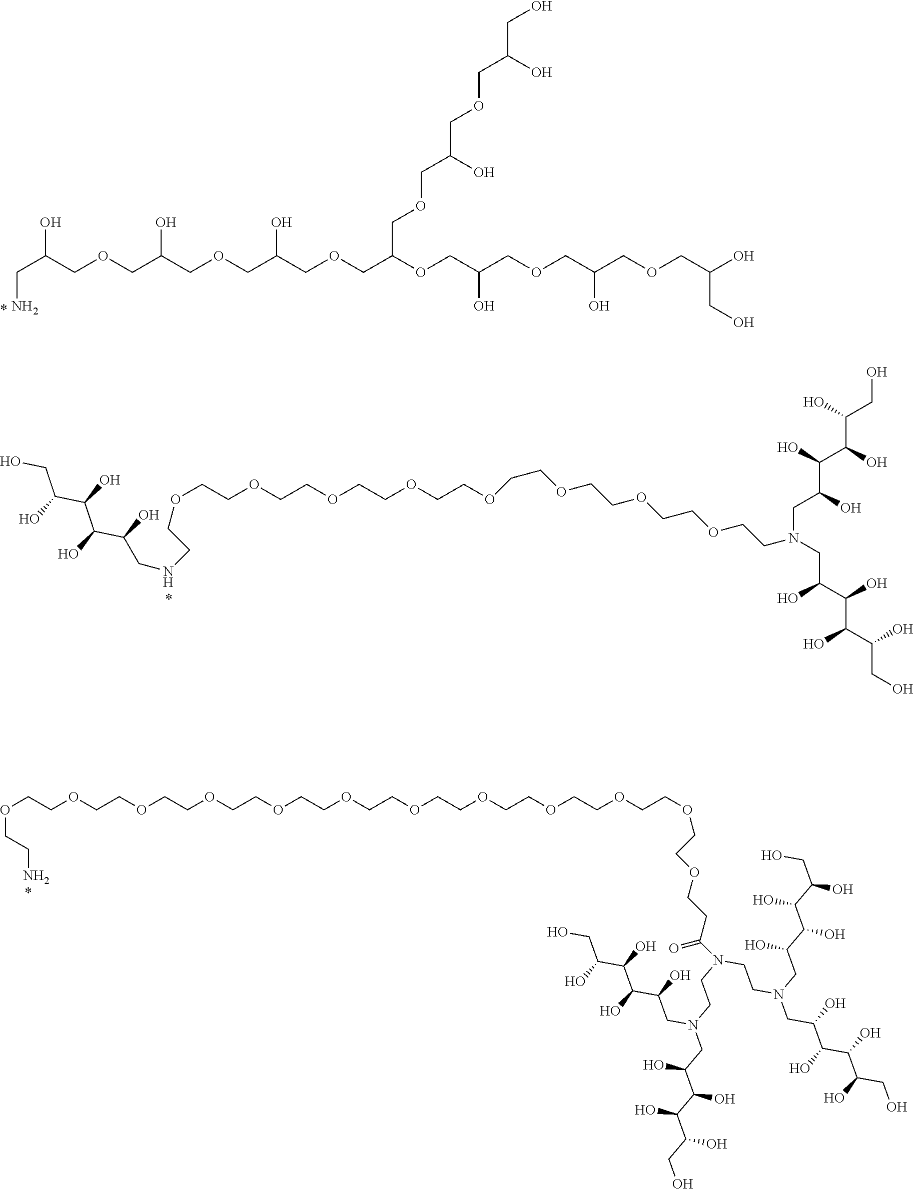 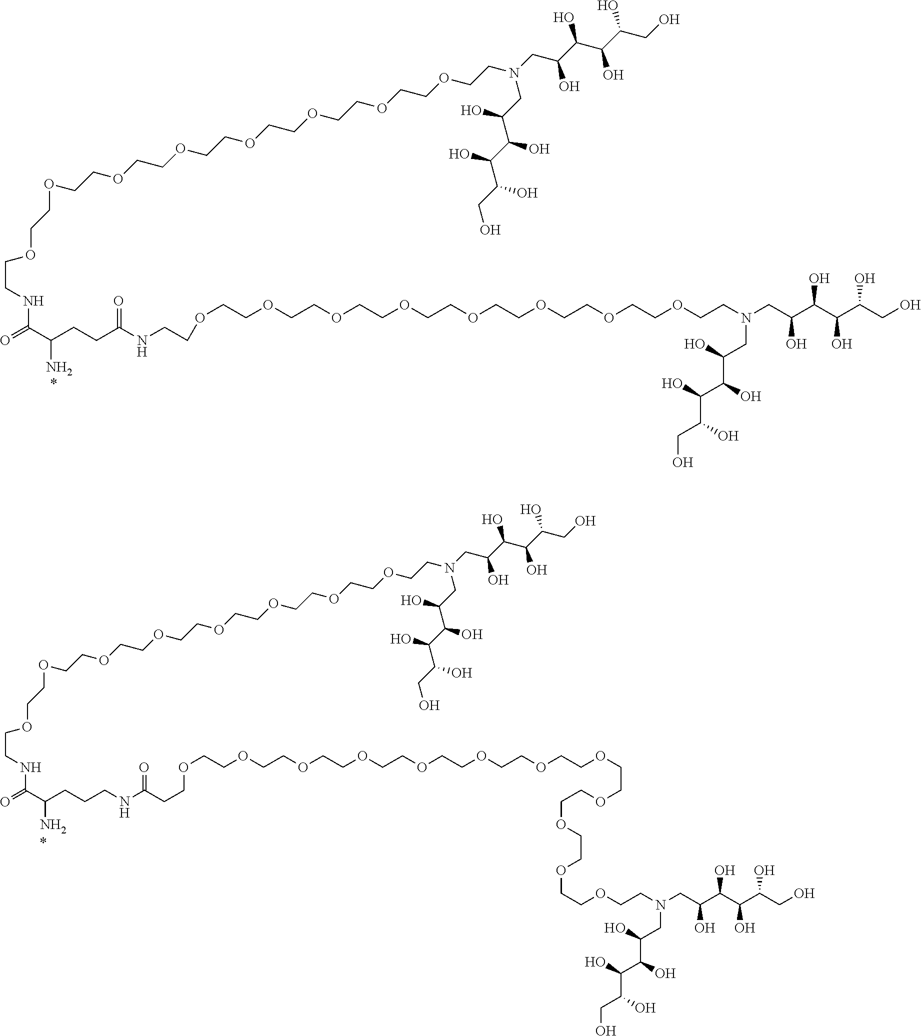 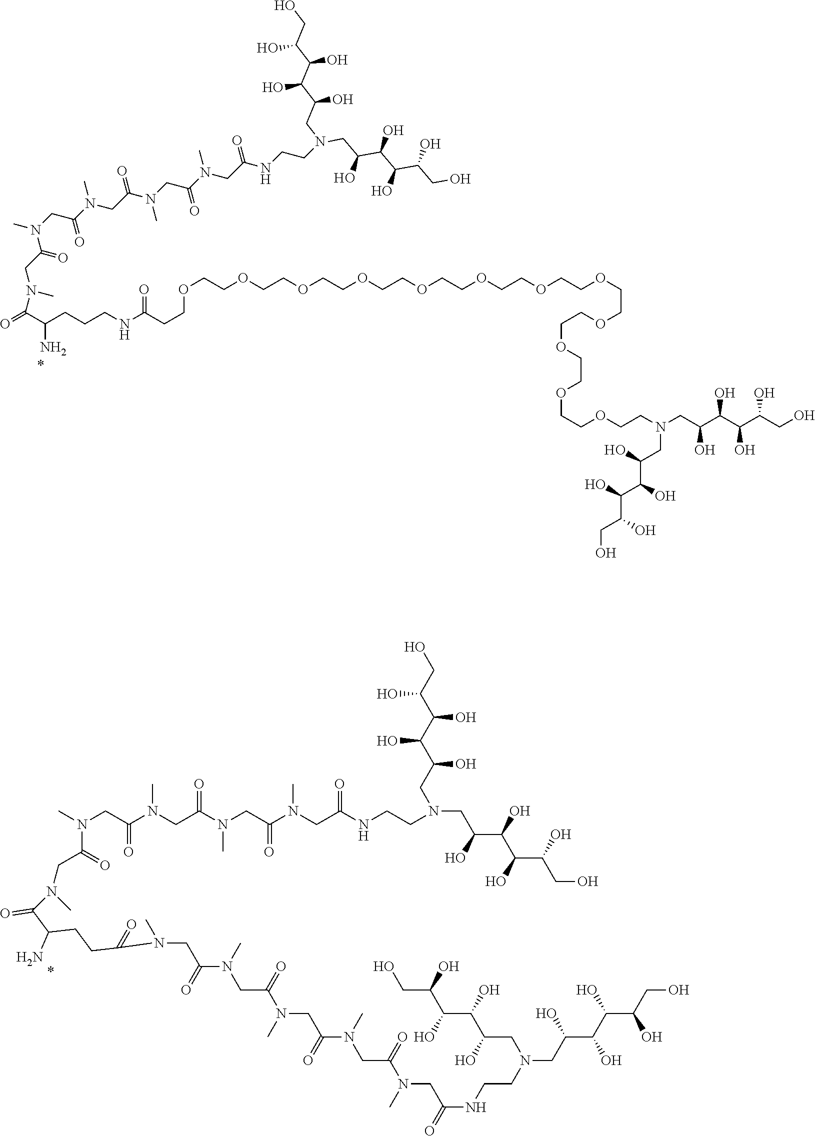 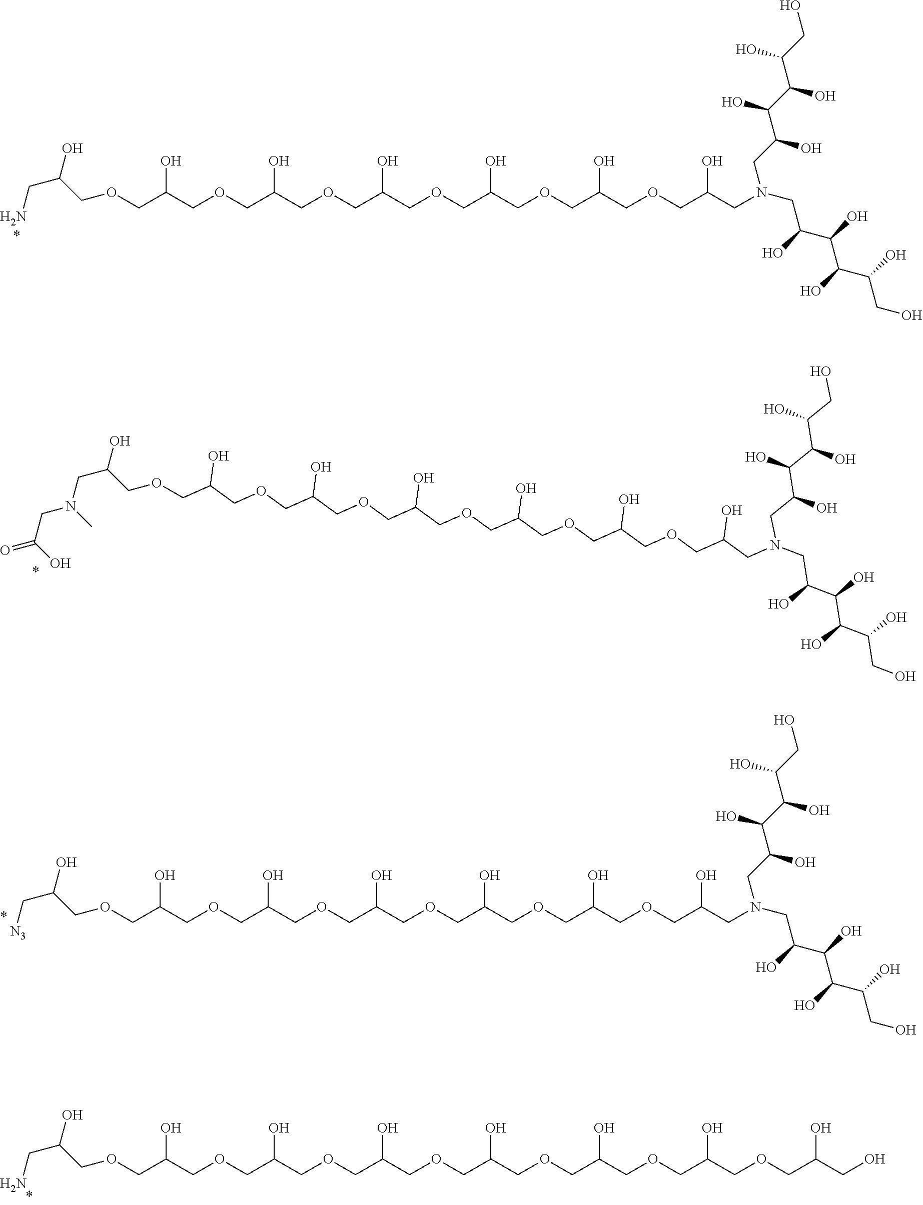 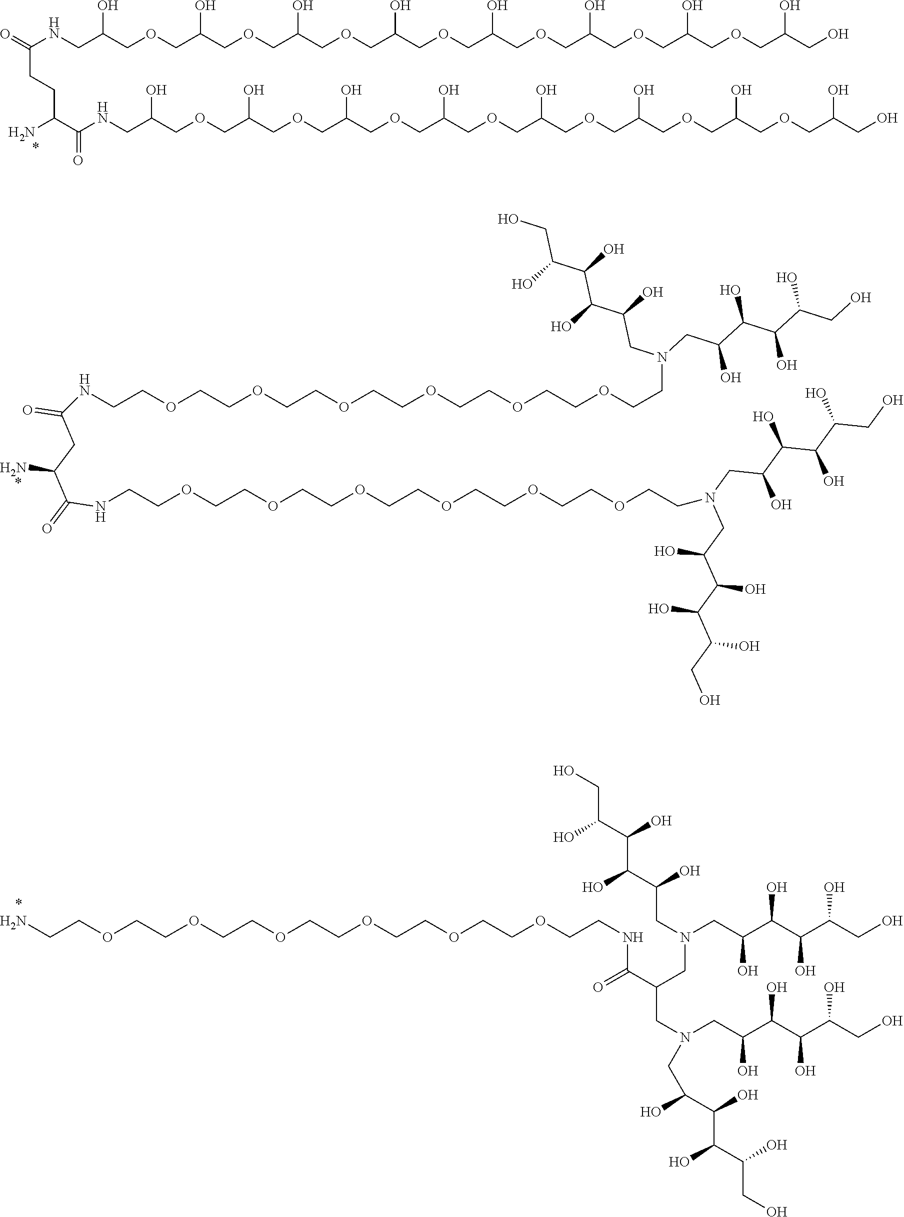 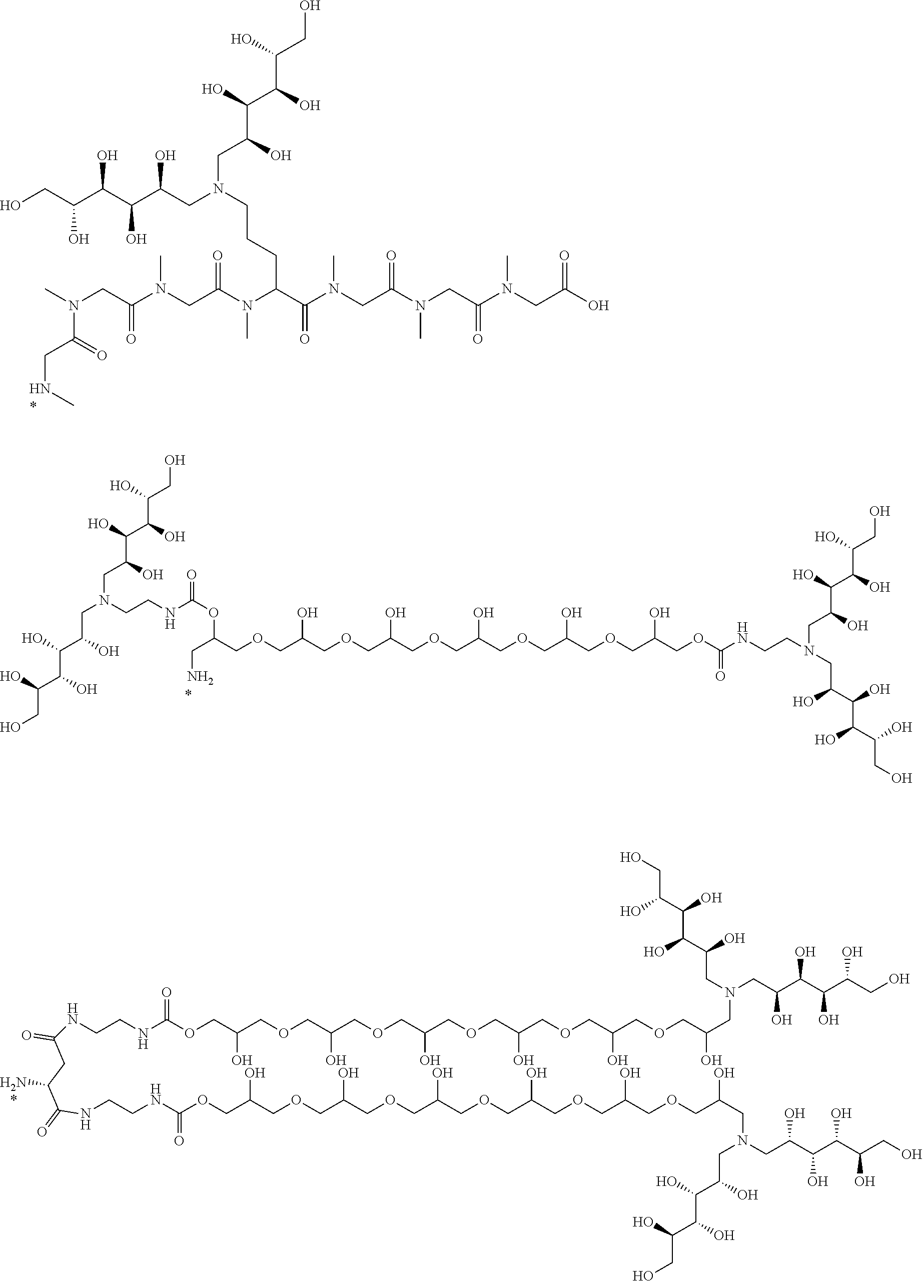 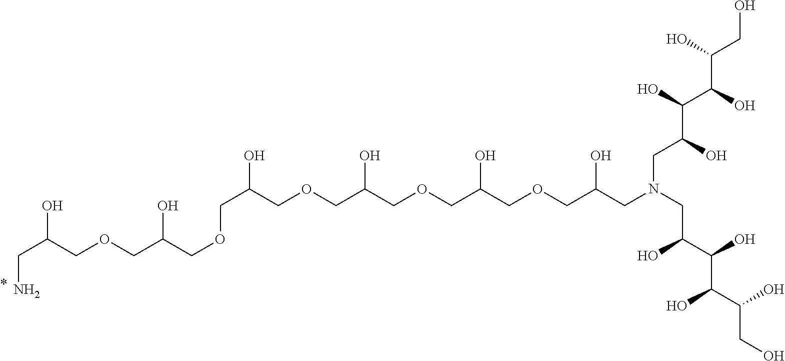 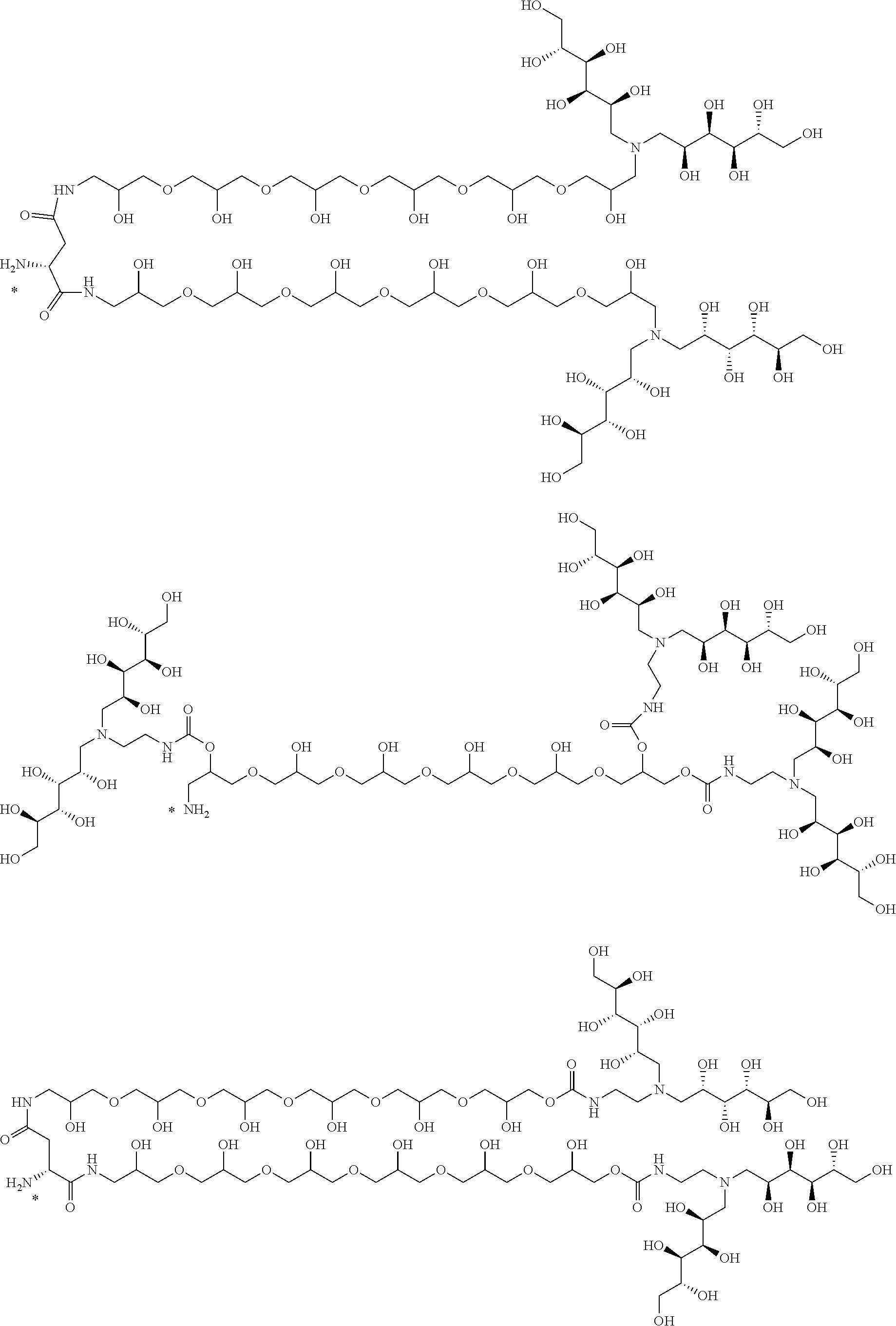 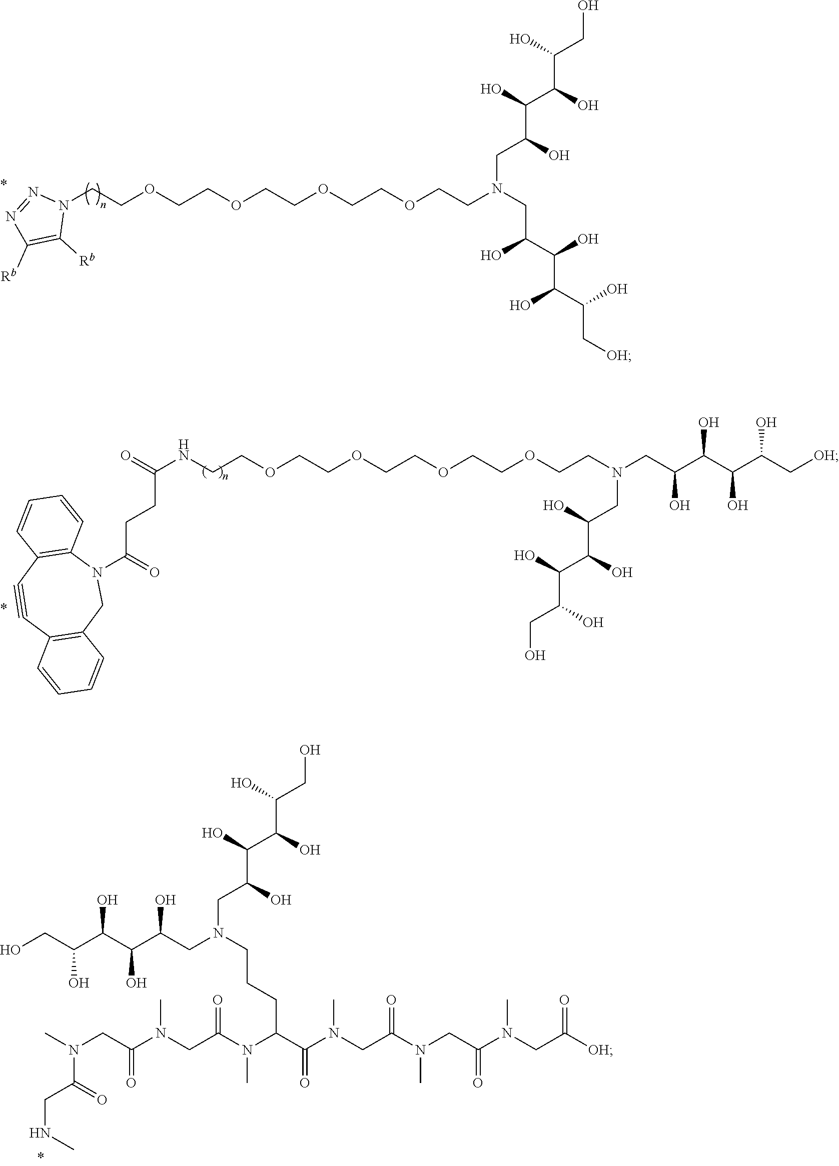 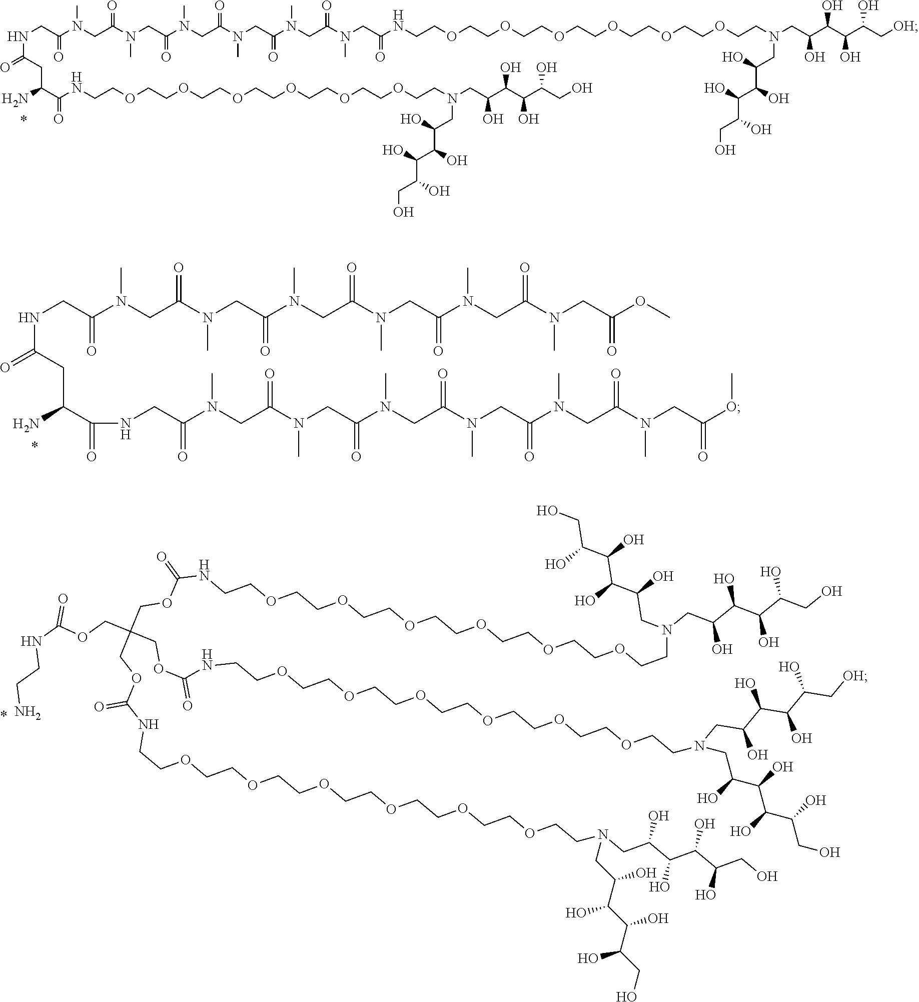 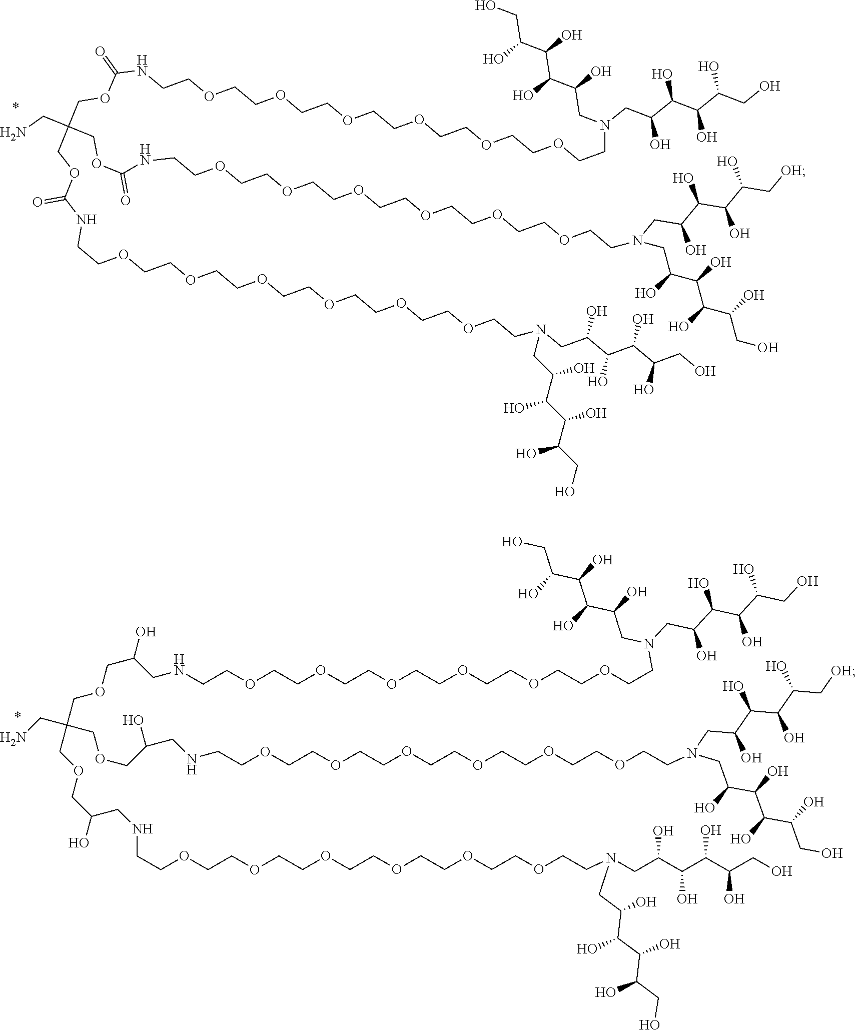 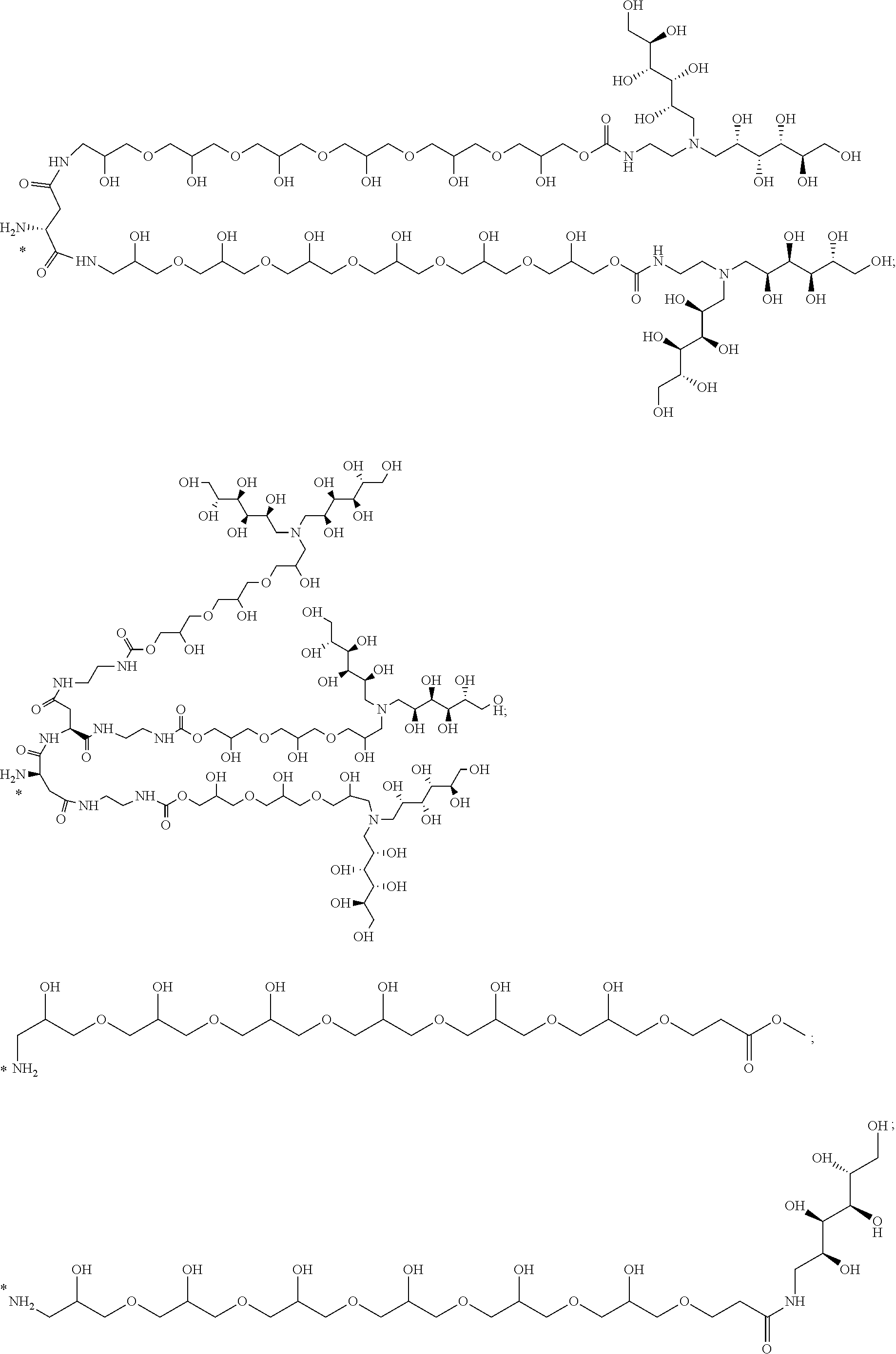 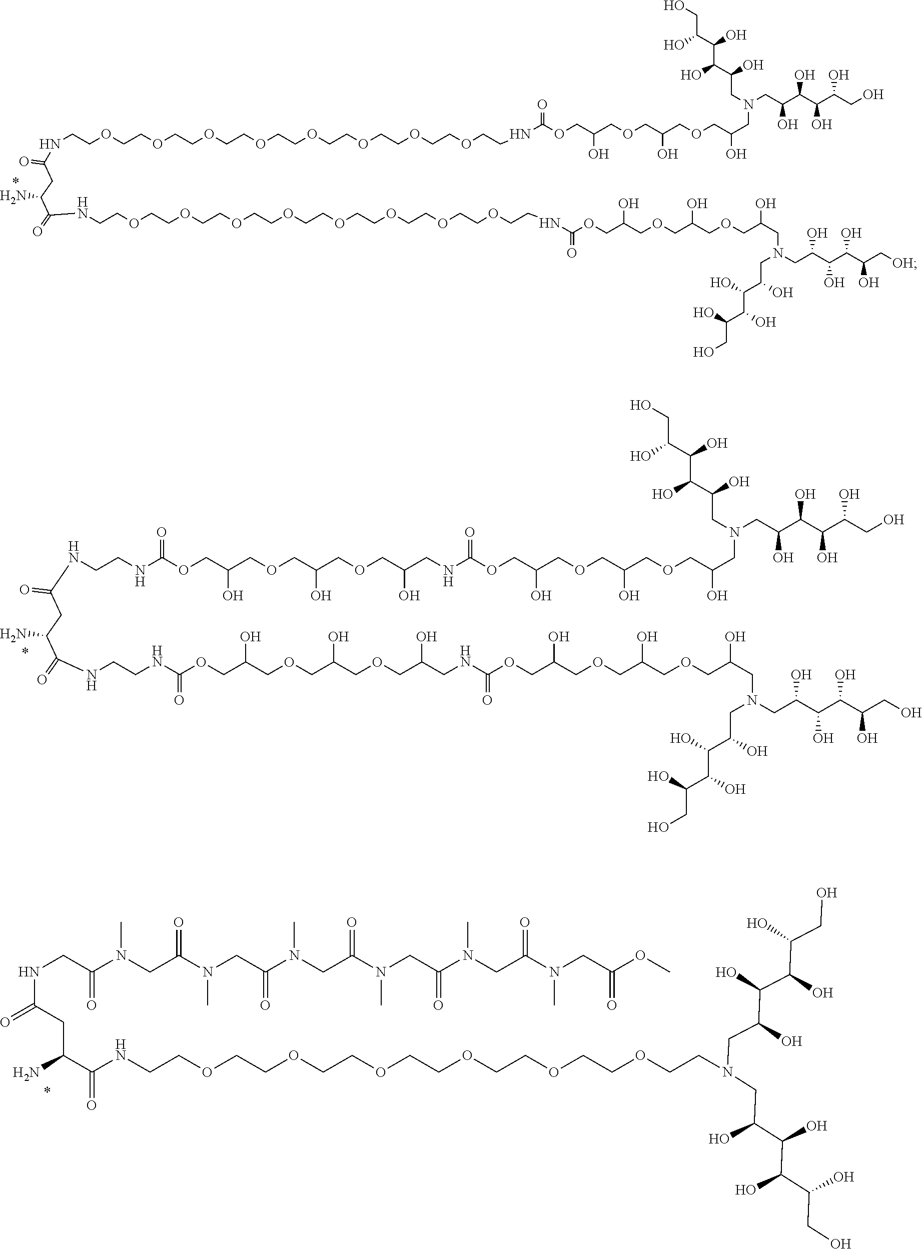 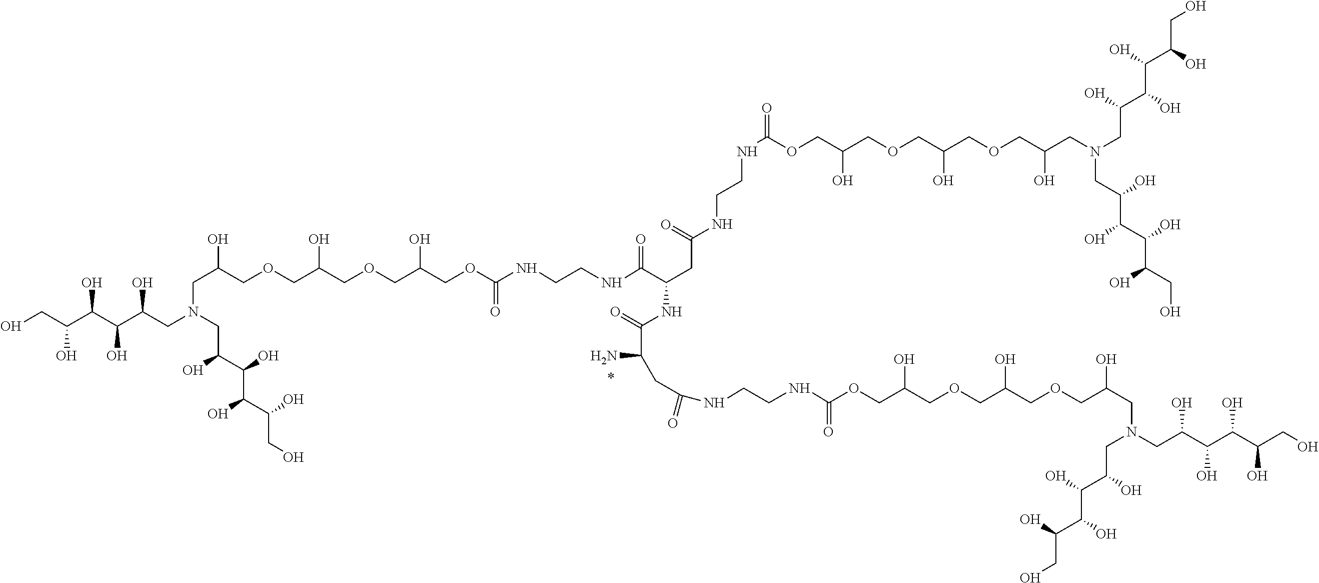 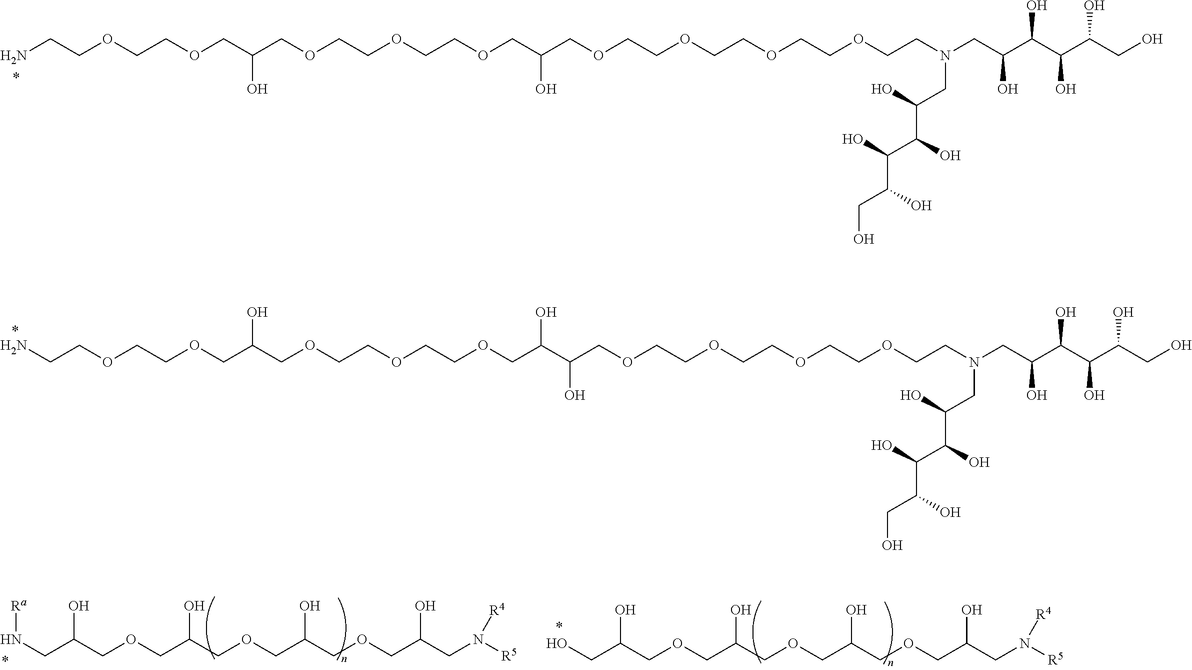 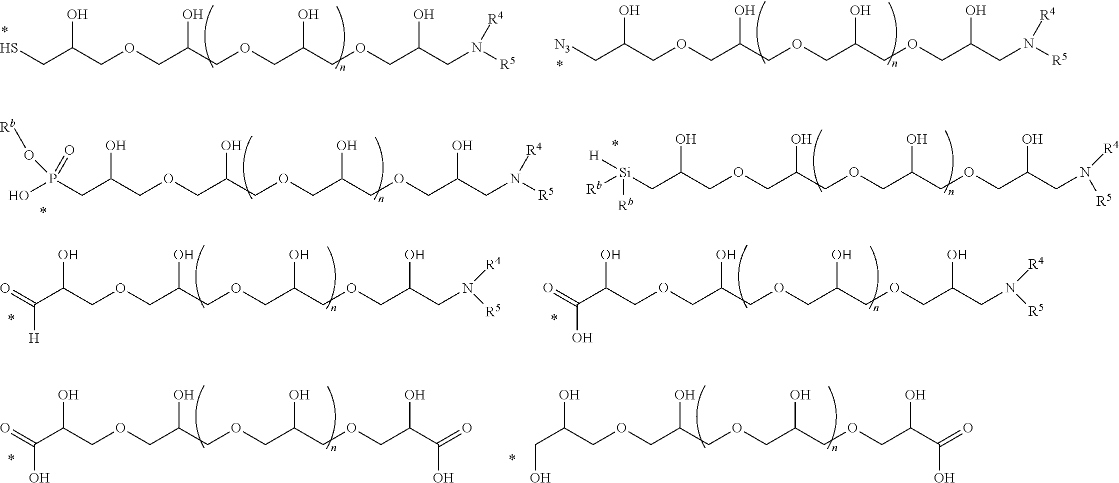 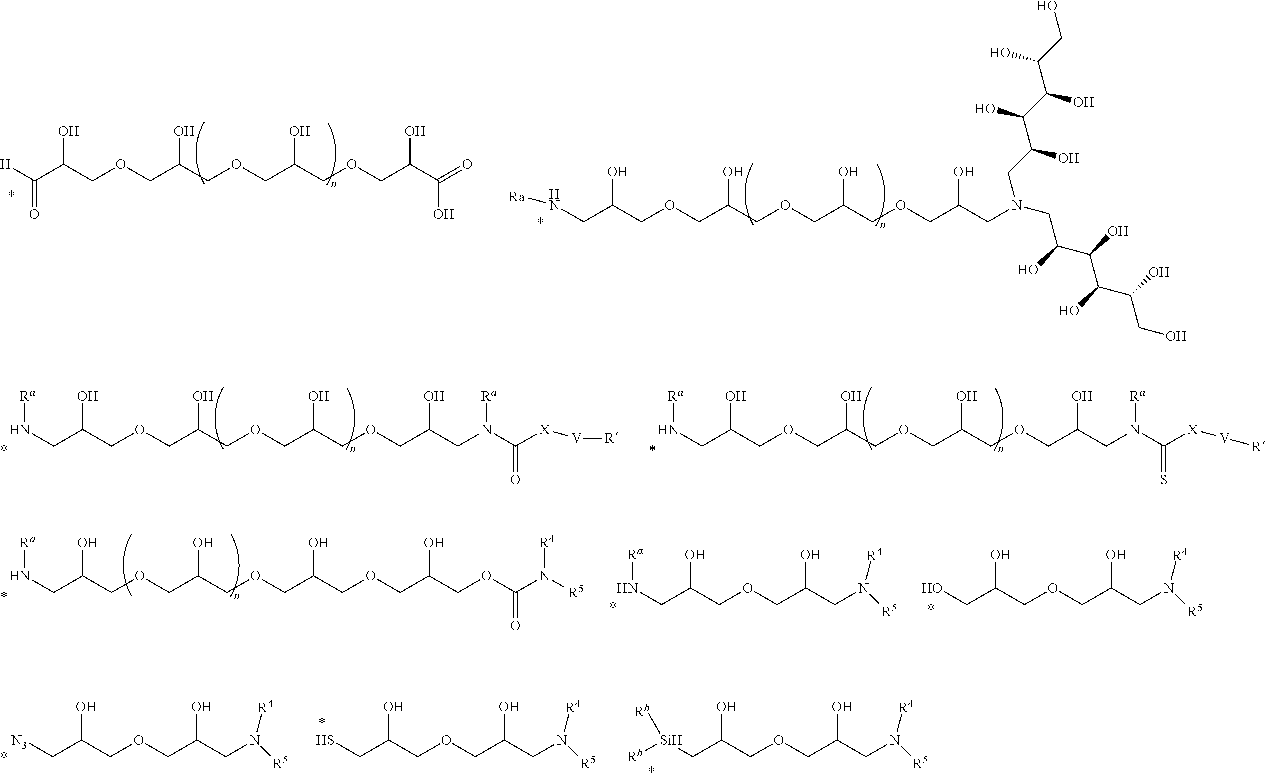 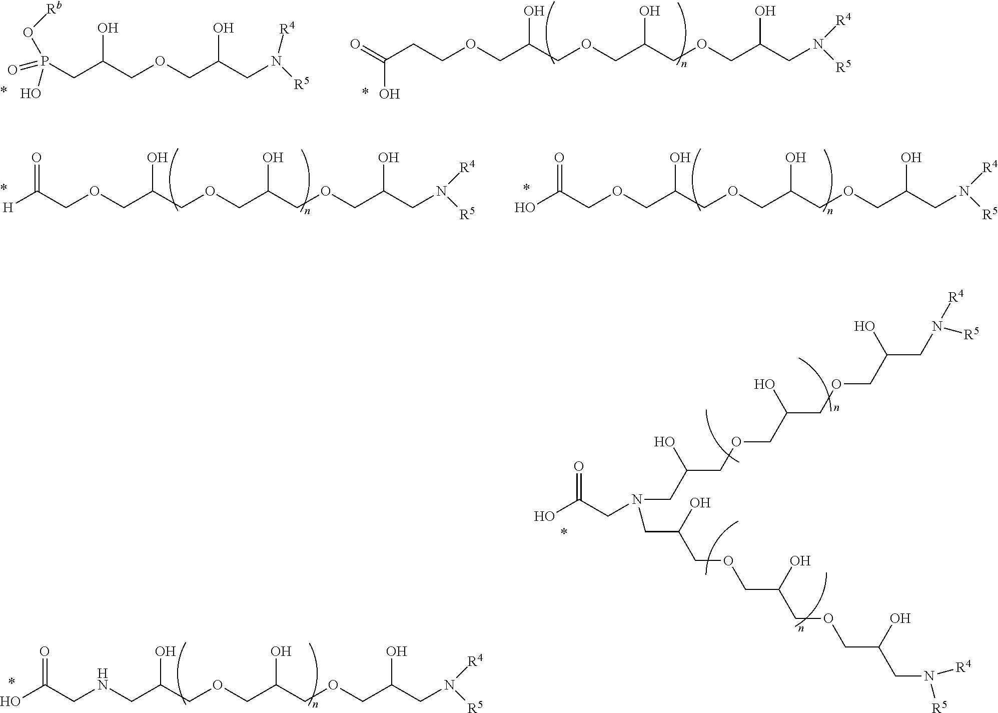 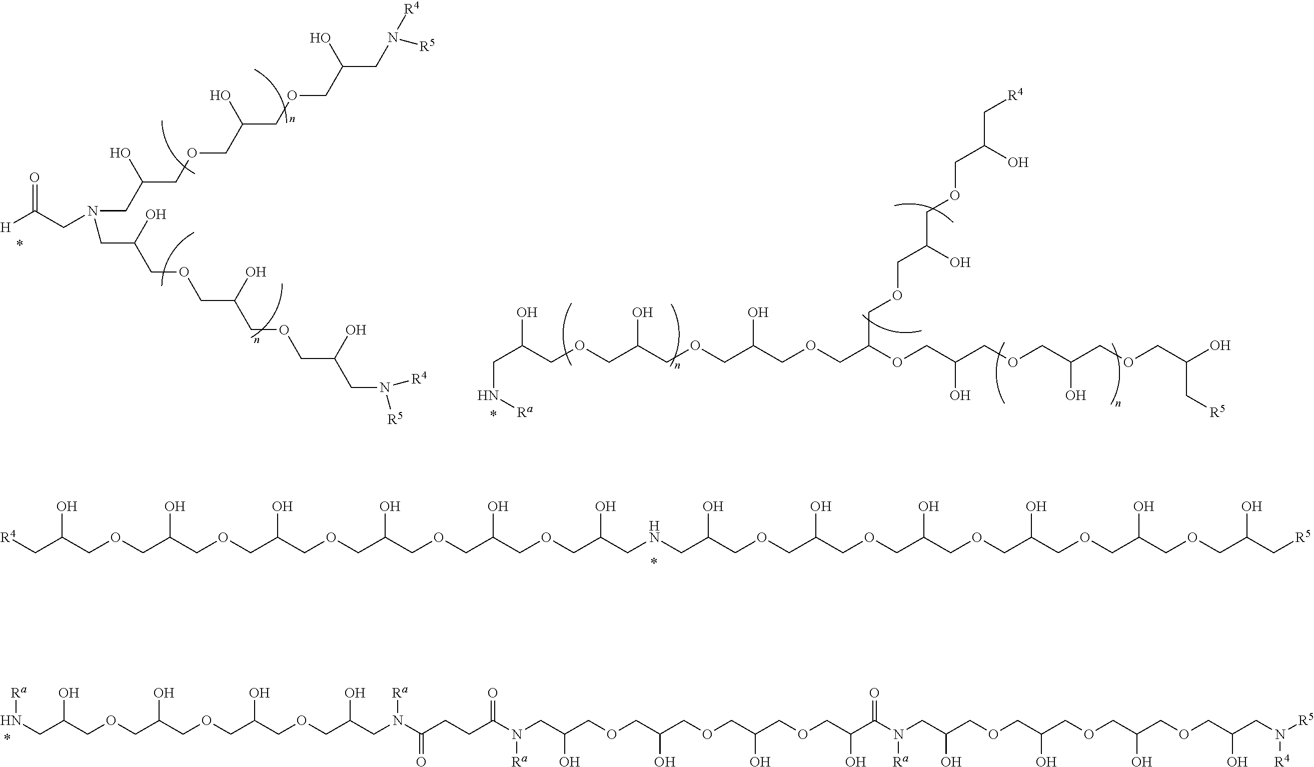 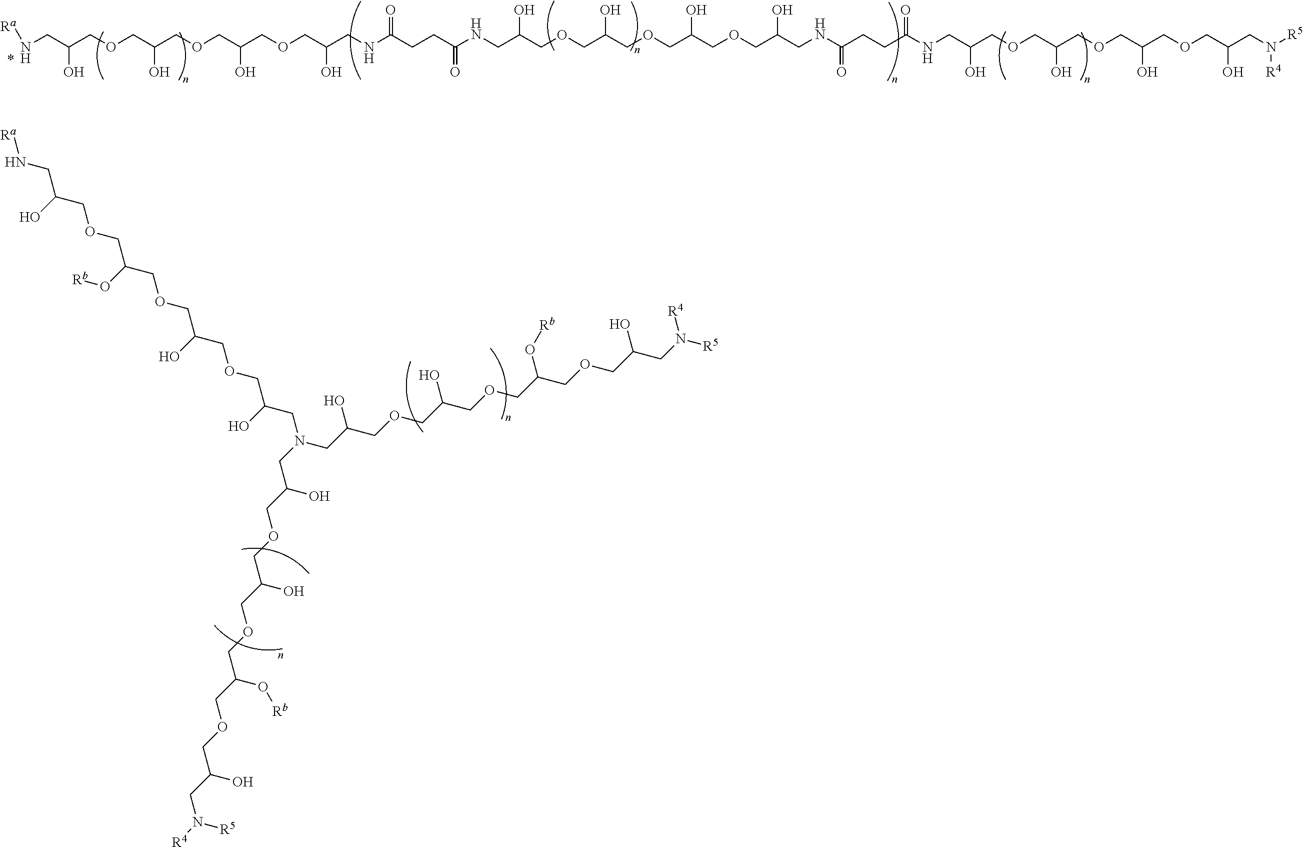 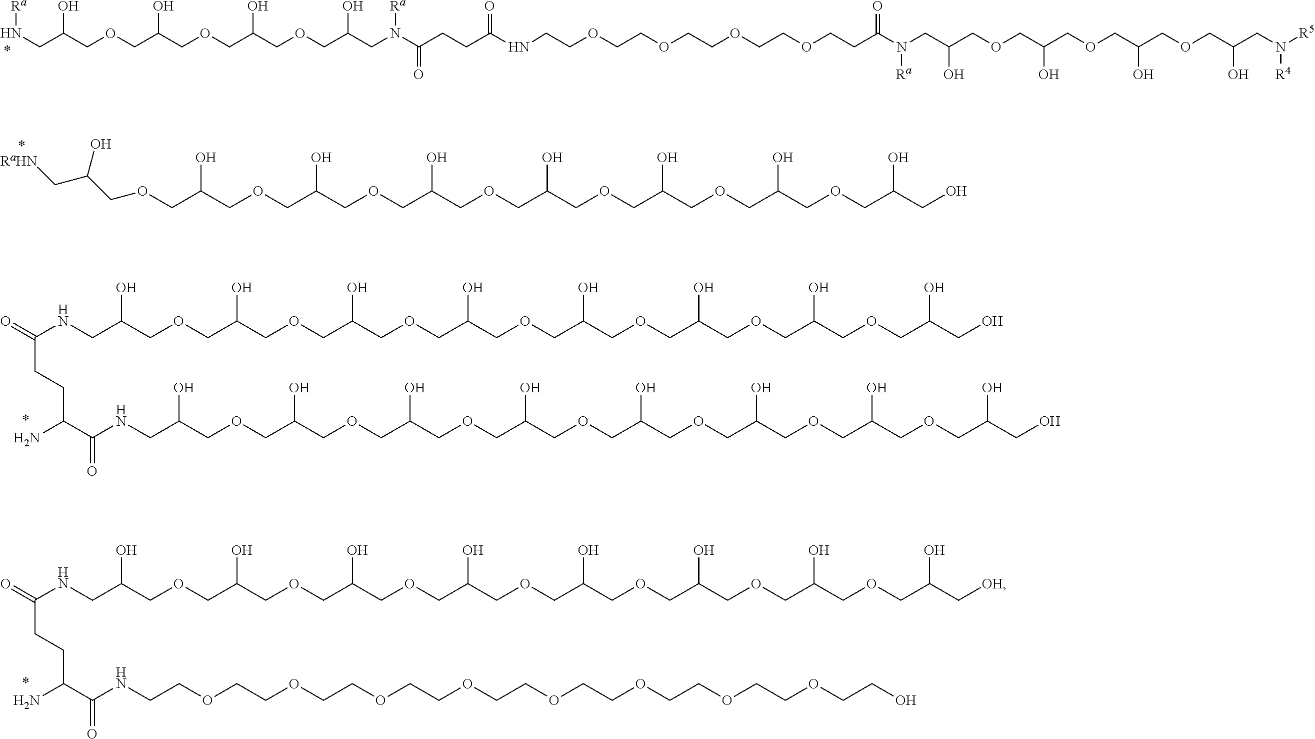 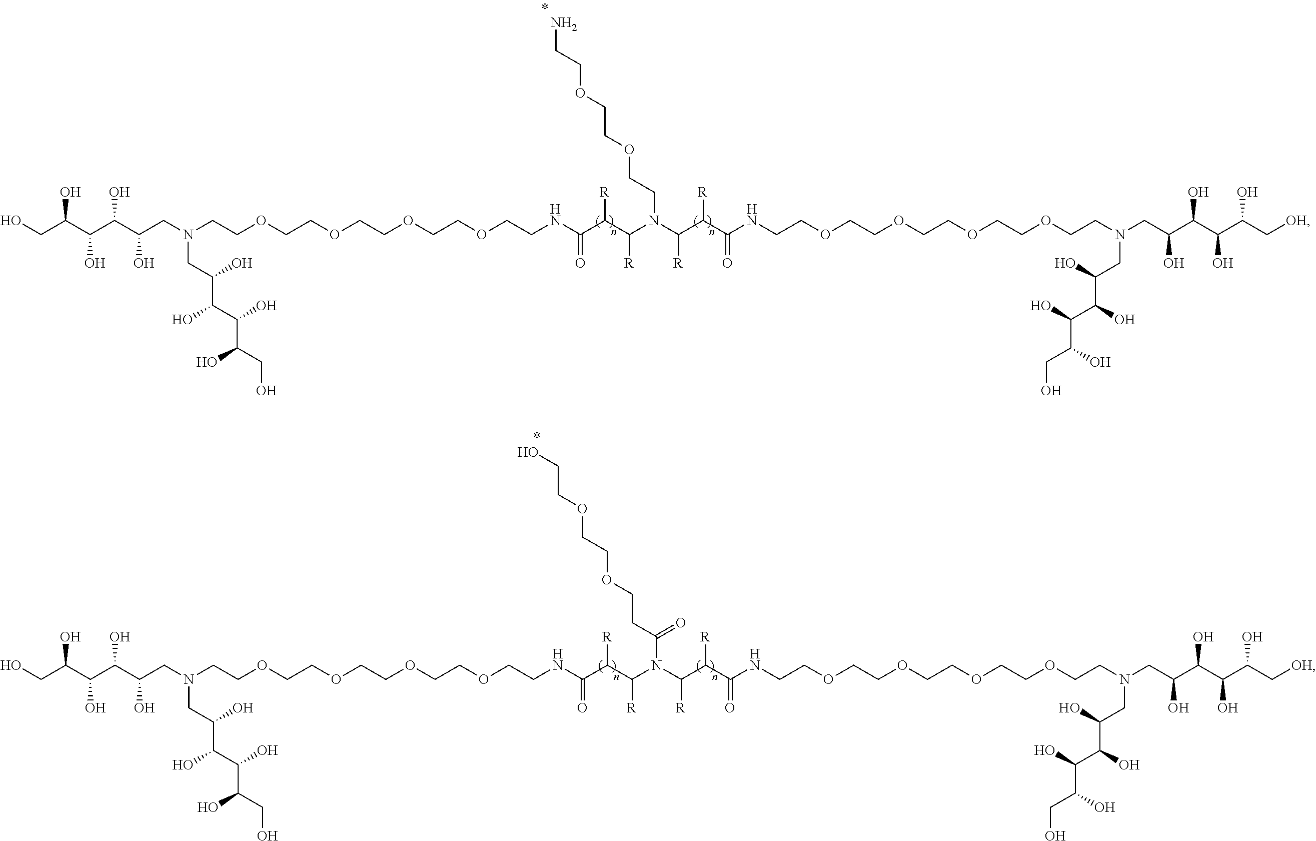 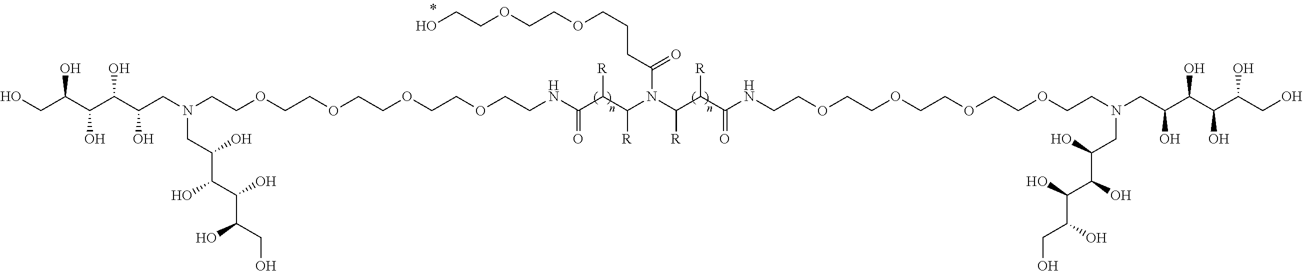
wherein:
(*) indicates the attachment site to site R^{b}, or to the enzyme-cleavable group;
each R is independently H or C₁-C₆ alkyl;
R′ is H, C₁-C₆ alkyl, —N(R²⁴)(R²⁵) or —CO₂H;
each n is independently 1 to 12;
X is 0, NR or —CH₂—;
V is bond or C₁-C₆ alkyl;
one of R²⁴ and R²⁵ is selected from a H; polyhydroxyl group; substituted polyhydroxyl group; —C(O)-polyhydroxyl group; substituted —C(O)-polyhydroxyl group; substituted —C(O)—C₁-C₈ alkyl; a chelator; and —C(O)—R²⁸, where R²⁸ is a Sugar unit of formula (XII) or (XIII); and the other of R²⁴ and R²⁵ is selected from H; polyhydroxyl group; substituted polyhydroxyl group; —C(O)-polyhydroxyl group; substituted —C(O)-polyhydroxyl group; substituted —C(O)—C₁-C₈ alkyl; a chelator; —C(O)—R²⁸, where R²⁸ is a Sugar unit of formula (XII) or (XIII); and polyethylene glycol, optionally having 1 to 24 ethylene glycol subunits, provided that R²⁴ and R²⁵ are not both H.

45. The Linker compound of any one of claims 1-6, comprising a Polar group having a formula selected from: 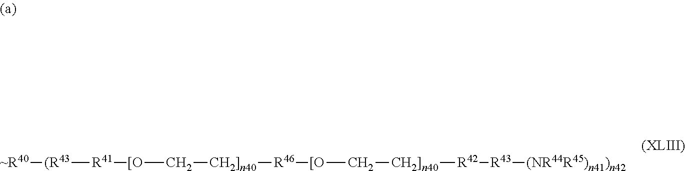
or a stereoisomer or salt thereof, wherein:
R⁴⁰ is an attachment group to site R^{b}, or to the enzyme-cleavable group;
R⁴¹ and R⁴² are each, independently, a bond or C₁-C₆ alkylene;
each R⁴³ is, independently, selected from a bond, C₁-C₁₂ alkylene, —OC₁-C₁₂ alkylene, —C(═O)—, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)—, —C(O)—C₁-C₁₂ alkylene-NH—, —NH—C(O)—NH—, —NH—C(O)—, —NH—C(O)—C₁-C₁₂ alkylene, —C(O)—NH—C₁-C₁₂ alkylene, C₁-C₁₂alkylene-NH—C(O)—, -heteroarylene, heteroaryl-C₁-C₁₂ alkylene, heteroaryl-C₁-C₁₂ alkylene-C(O)—, or —C(O)NR⁴⁶R⁴⁷, wherein one of R⁴⁶ and R⁴⁷ is H or C₁-C₁₂ alkylene and the other is C₁-C₁₂ alkylene;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, provided that R⁴⁴ and R⁴⁵ are not both H;
each R⁴⁶ is independently selected from —NR⁵⁰—, —NR⁵⁰—C₁-C₆alkylene-NR⁵⁰—, —NR⁵⁰—C(O)—NR⁵⁰—S(O)₂—NR⁵⁰— or —NR⁵⁰—C(O)—C₁₋₆alkylene-;
each R⁵⁰ is independently selected from H, C₁-C₆ alkyl, or polyhydroxyl group;
each n40 is independently 2 to 26;
n41 is 1 to 6; and
n42 is 1 to 6; 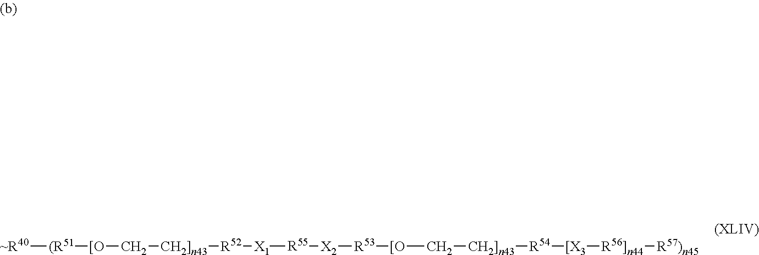
or a stereoisomer or salt thereof, wherein:
R⁴⁰ is an attachment group to site R^{b}, or to the enzyme-cleavable group;
R⁵¹, R⁵², R⁵³ and R⁵⁴ are each, independently, a bond or C₁-C₆ alkylene;
X₁, X₂ and X₃ are each independently —NR^{N}—C(O)— or —C(O)—NR^{N}—;
each R^{N} independently represent H, C₁-C₆ alkyl, or polyhydroxyl group;
R⁵⁵ and R⁵⁶ each independently represent a bivalent polyhydroxyl group;
R⁵⁷ is H, OH or C₁-C₆ alkyl;
each n43 is independently 0 to 26, with the proviso that at least one n43 is 1 to 26;
n44 is 0 to 10; and
n45 is 1 or 2; or 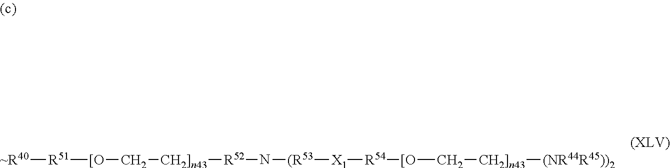
or a stereoisomer or salt thereof, wherein:
R⁴⁰ is an attachment group to site R^{b}, or to the enzyme-cleavable group;
R⁵¹, R⁵³ and R⁵⁴ are each, independently, a bond or optionally-substituted C₁-C₆ alkylene;
R⁵² is a bond, C₁-C₆ alkylene, —C(O)— or —O—C(O)—;
each X₁ is independently —NR^{N}—C(O)— or —C(O)—NR^{N}—;
each R^{N} independently represent H, C₁-C₆ alkyl, or polyhydroxyl group;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, provided that R⁴⁴ and R⁴⁵ are not both H; and
each n43 is independently 2 to 26.

46. The Linker compound of claim 45, comprising a Polar group having one of the following structures prior to attachment to the enzyme-cleavable group and/or to the Linker Unit: 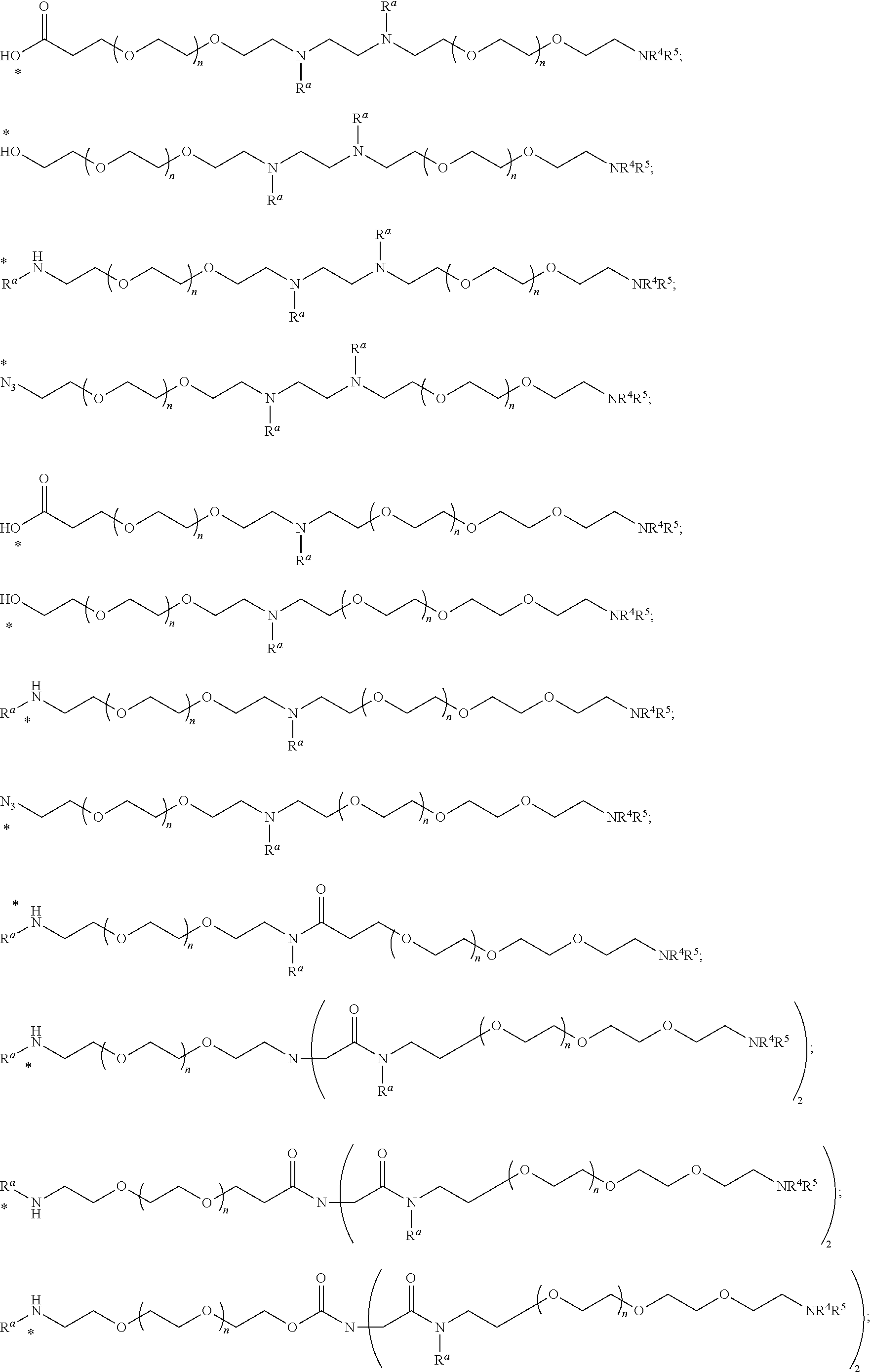 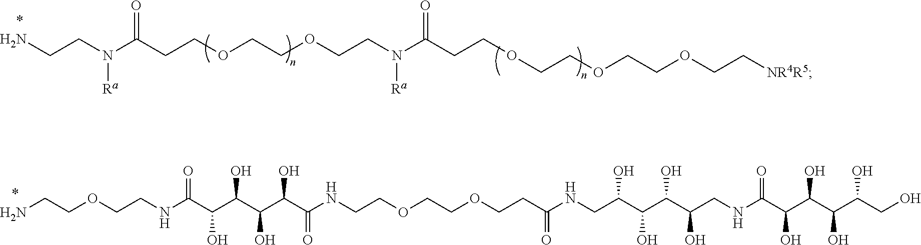
wherein:
(*) indicates the attachment site to site R^{b}, or to the enzyme-cleavable group;
each R is independently H, alkyl or polyhydroxyl group;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, provided that R⁴⁴ and R⁴⁵ are not both H; and
each n is independently 1 to 12.

47. The Linker compound of any one of claims 1-6, comprising a Polar group having a formula selected from: 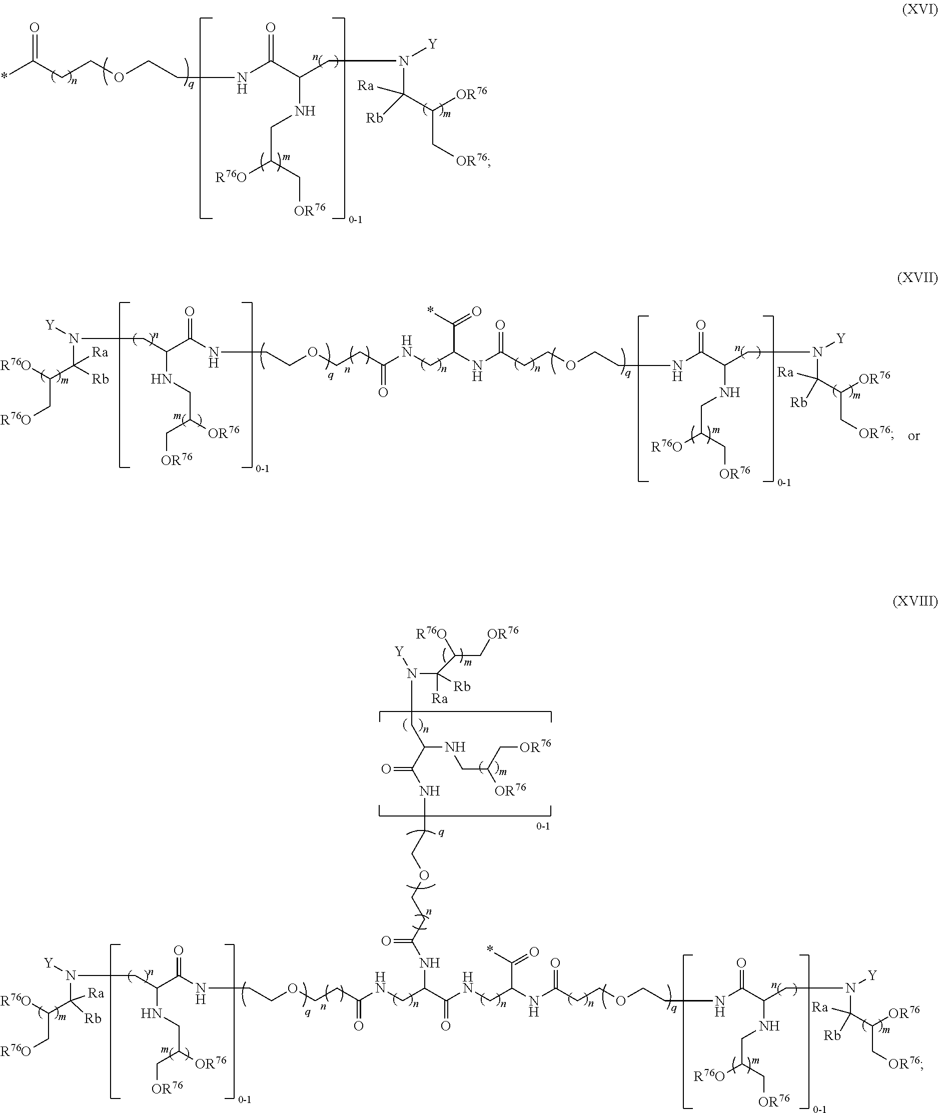
or a stereoisomer or salt thereof, wherein:
each Y is independently R⁷⁶ or 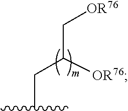
each R⁷⁶ is independently H, acetyl, —P(═O)(OH)₂, or —(CH₂)ᵥ—O—S(═O)₂(OH);
each Rₐ and R_{b} is independently H or Rₐ and R_{b} are taken together with the carbon to which they are attached to form an oxo group;
each q is independently 2-26;
each m is independently 1 to 4;
each n is independently 1 to 4;
each v is independently 1 to 6; and
each * is an attachment site to R^{b}, or to the enzyme-cleavable group.

48. The Linker compound of any one of claims 1-6, comprising a Polar group having a formula selected from: 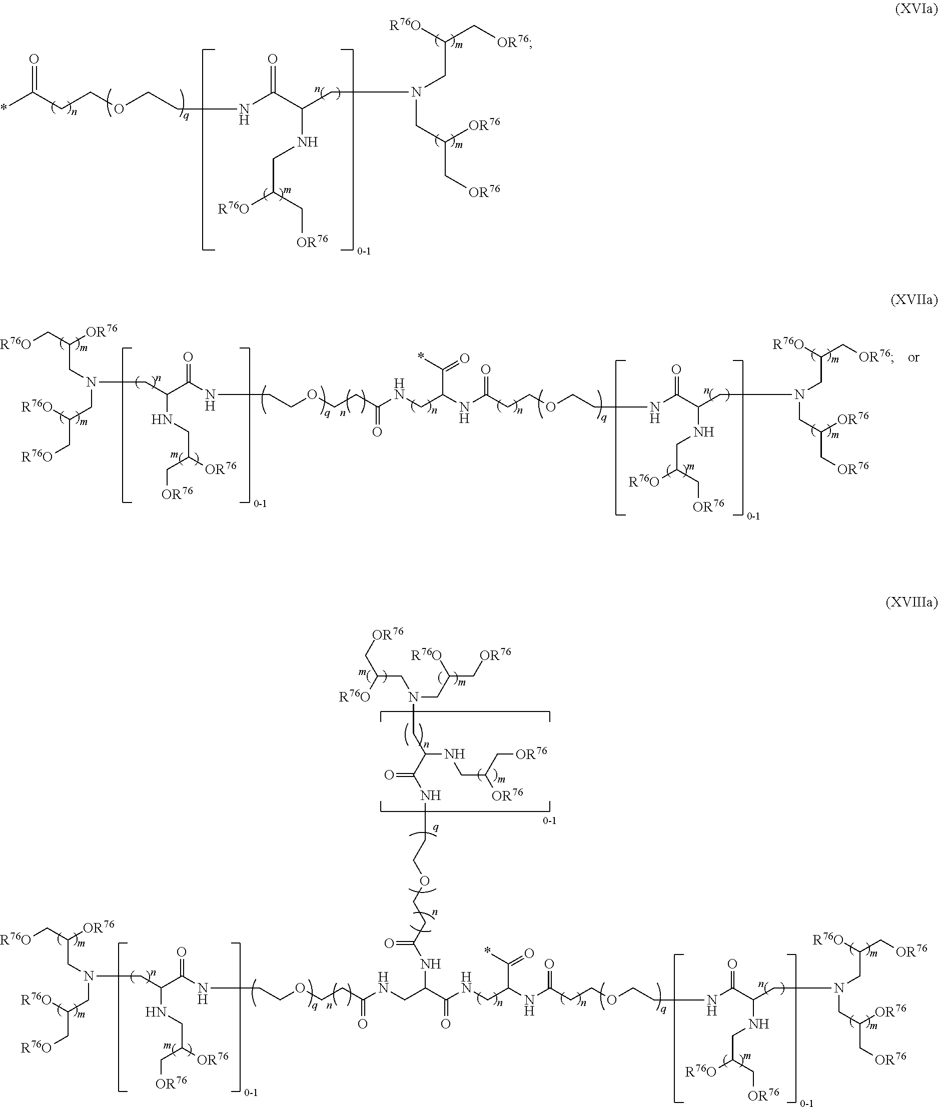
or a stereoisomer or salt thereof, wherein:
each R⁷⁶ is independently H, acetyl, —P(═O)(OH)₂, or —(CH₂)ᵥS(═O)₂(OH); P
each q is independently 2-26;
each m is independently 1 to 4;
each n is independently 1 to 4;
each v is independently 1 to 6; and
each * is an attachment site to R^{b}, or to the enzyme-cleavable group.

49. The Linker compound of any one of claims 1-6, comprising a Polar group having a formula selected from: 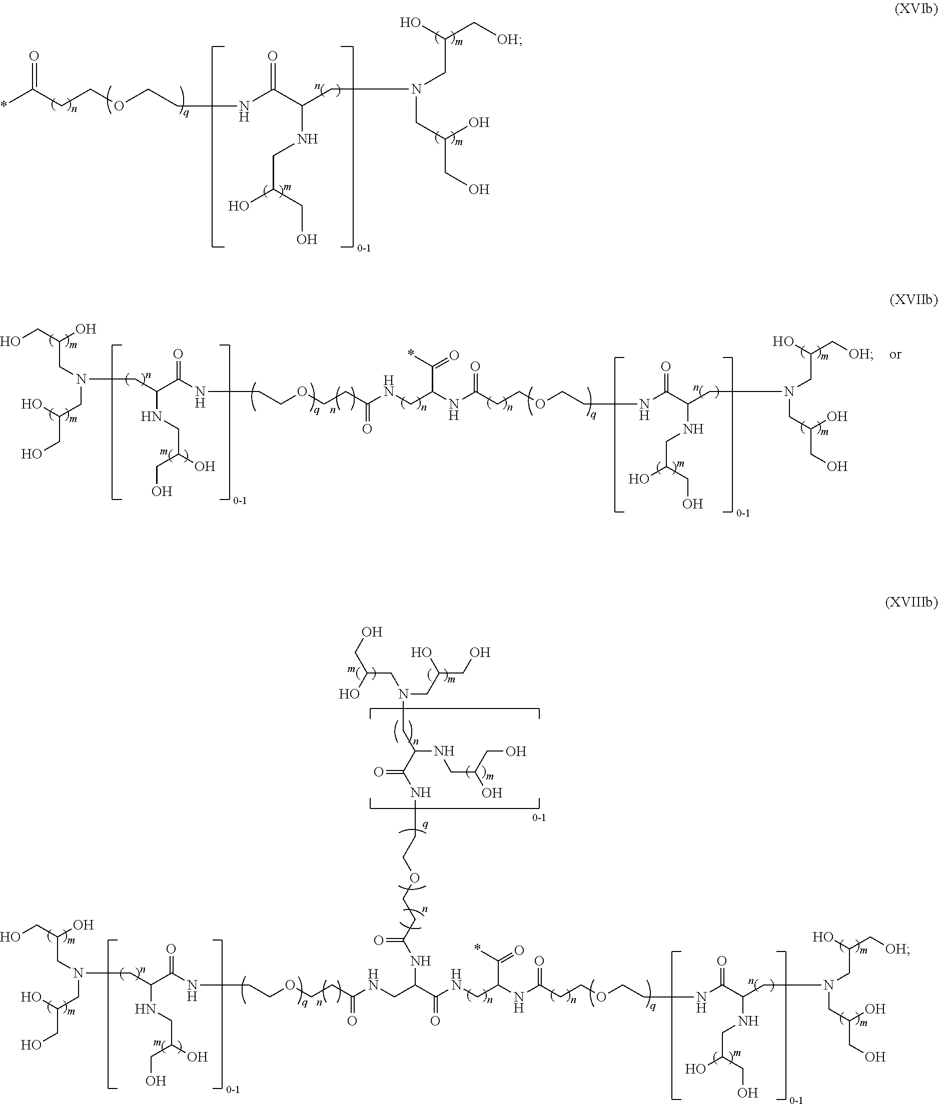
or a stereoisomer or salt thereof, wherein:
each q is independently 2-26;
each m is independently 1 to 4;
each n is independently 1 to 4; and
each * is an attachment site to R^{b}, or to the enzyme-cleavable group.

50. The Linker compound of claim 47, wherein Y is R⁷⁶.

51. The Linker compound of claim 47, wherein Y is

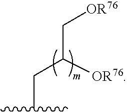

52. The Linker compound of claim 47, wherein each Rₐ and R_{b} is independently H.

53. The Linker compound of claim 47, wherein Rₐ and R_{b} are taken together with the carbon to which they are attached to form an oxo group.

54. The Linker compound of any one of claims 45-47, wherein q is 10-20.

55. The Linker compound of any one of claims 57-49, wherein q is 12.

56. The Linker compound of any one of claims 1-6, 35-37, or 47-49, comprising a Polar group selected from the following, or a stereoisomer or salt thereof: 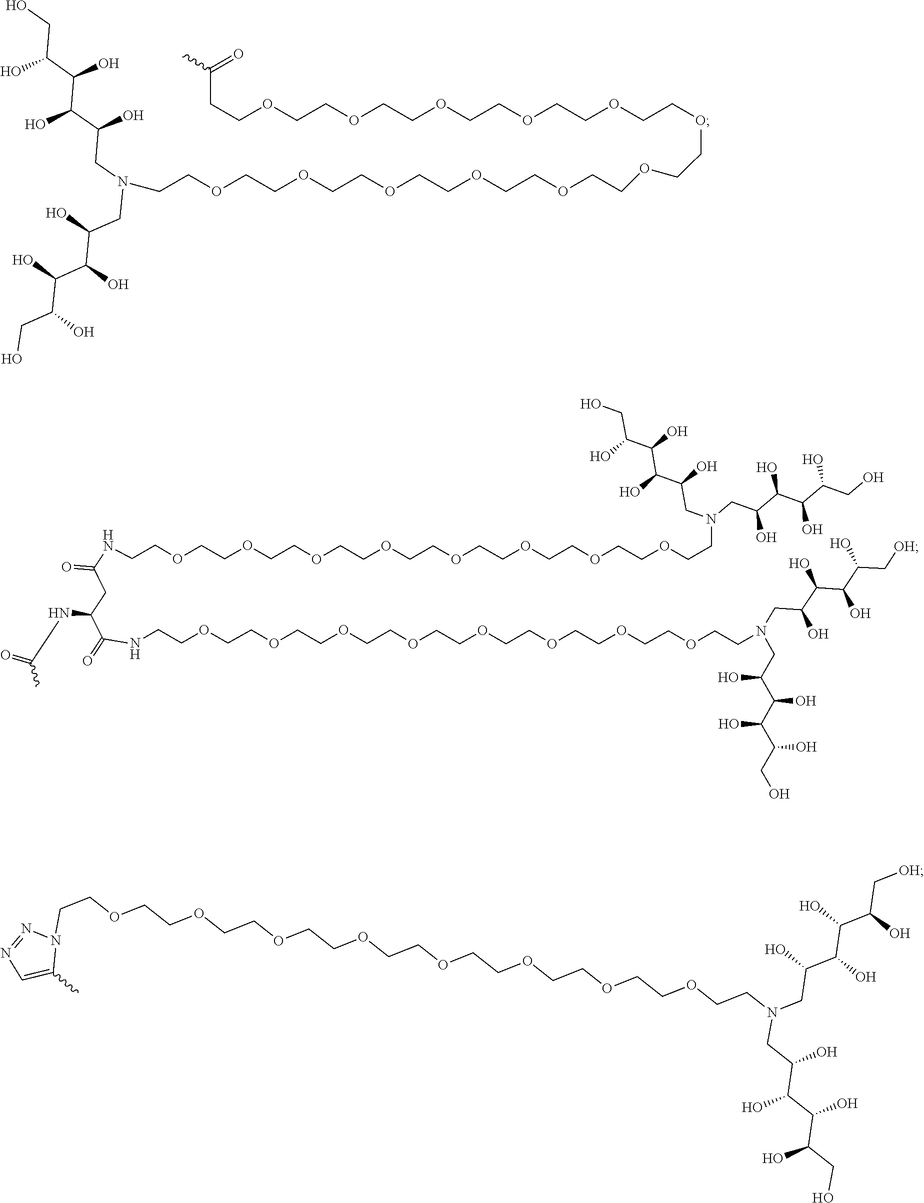 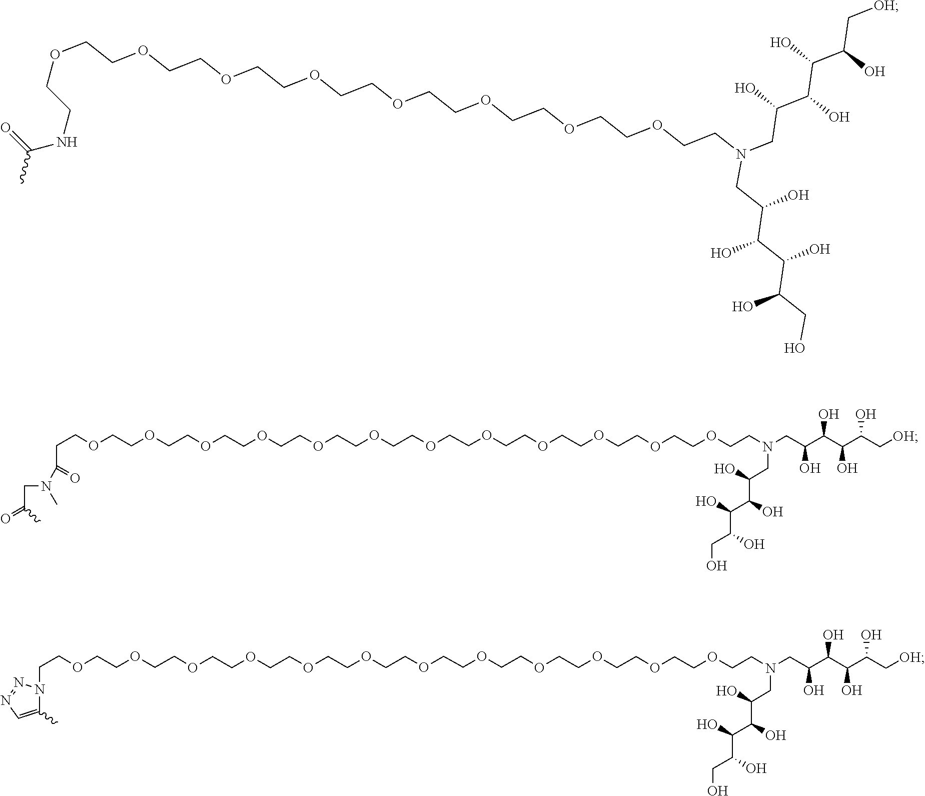 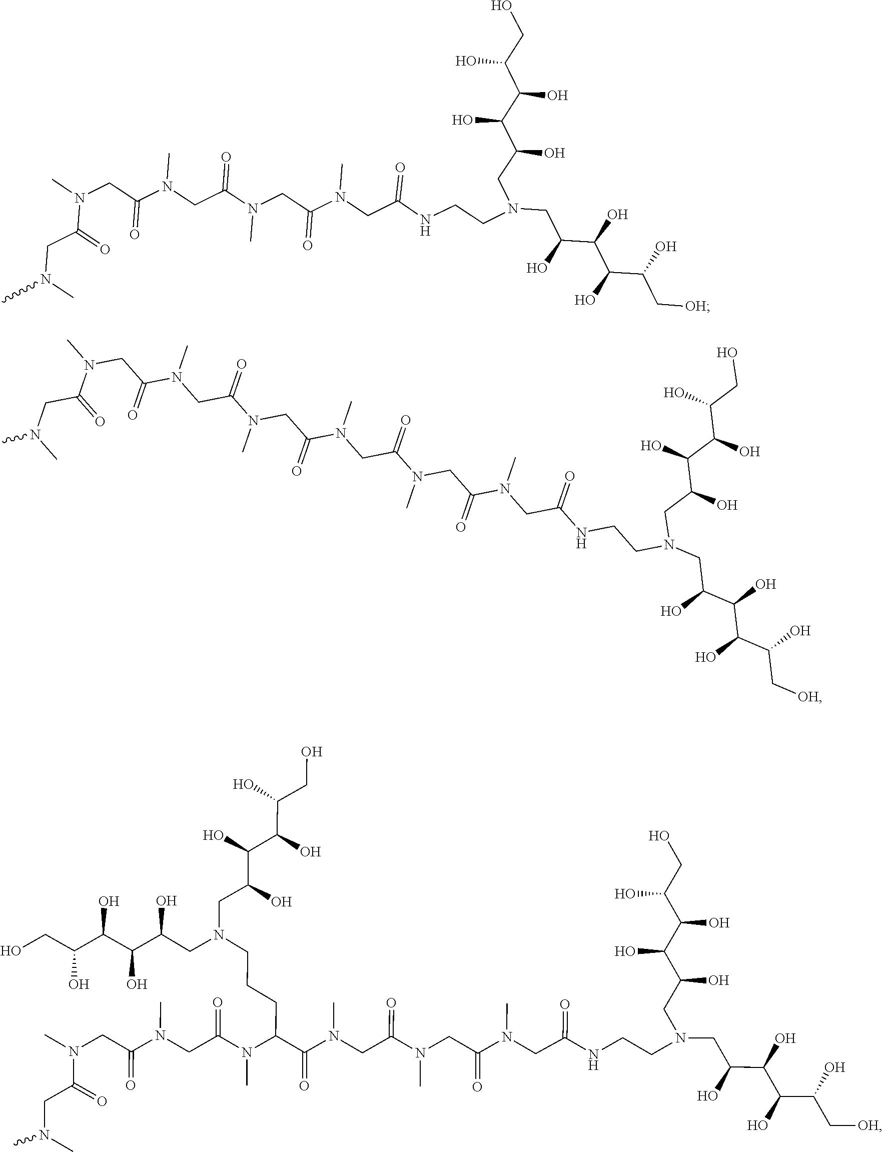 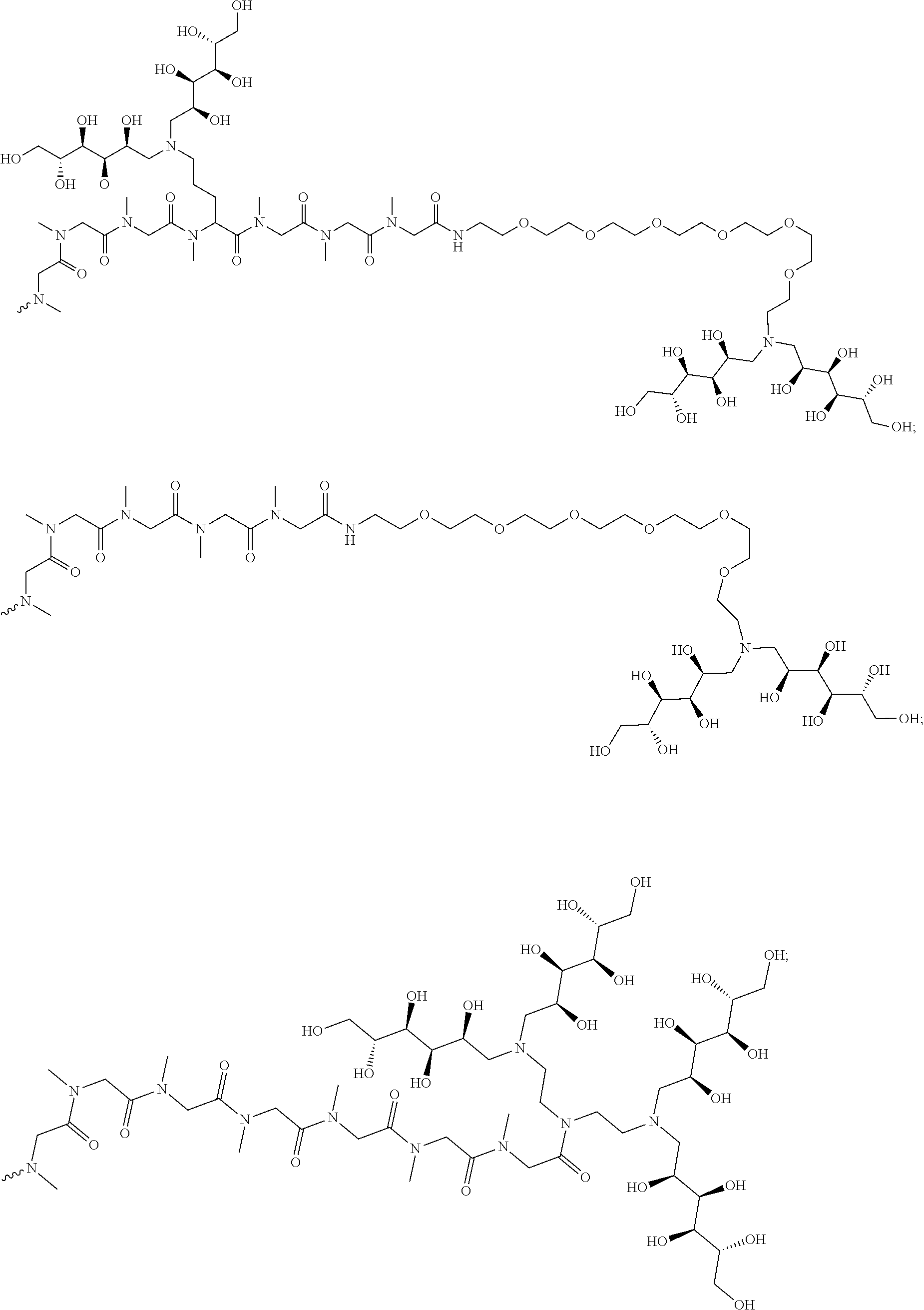 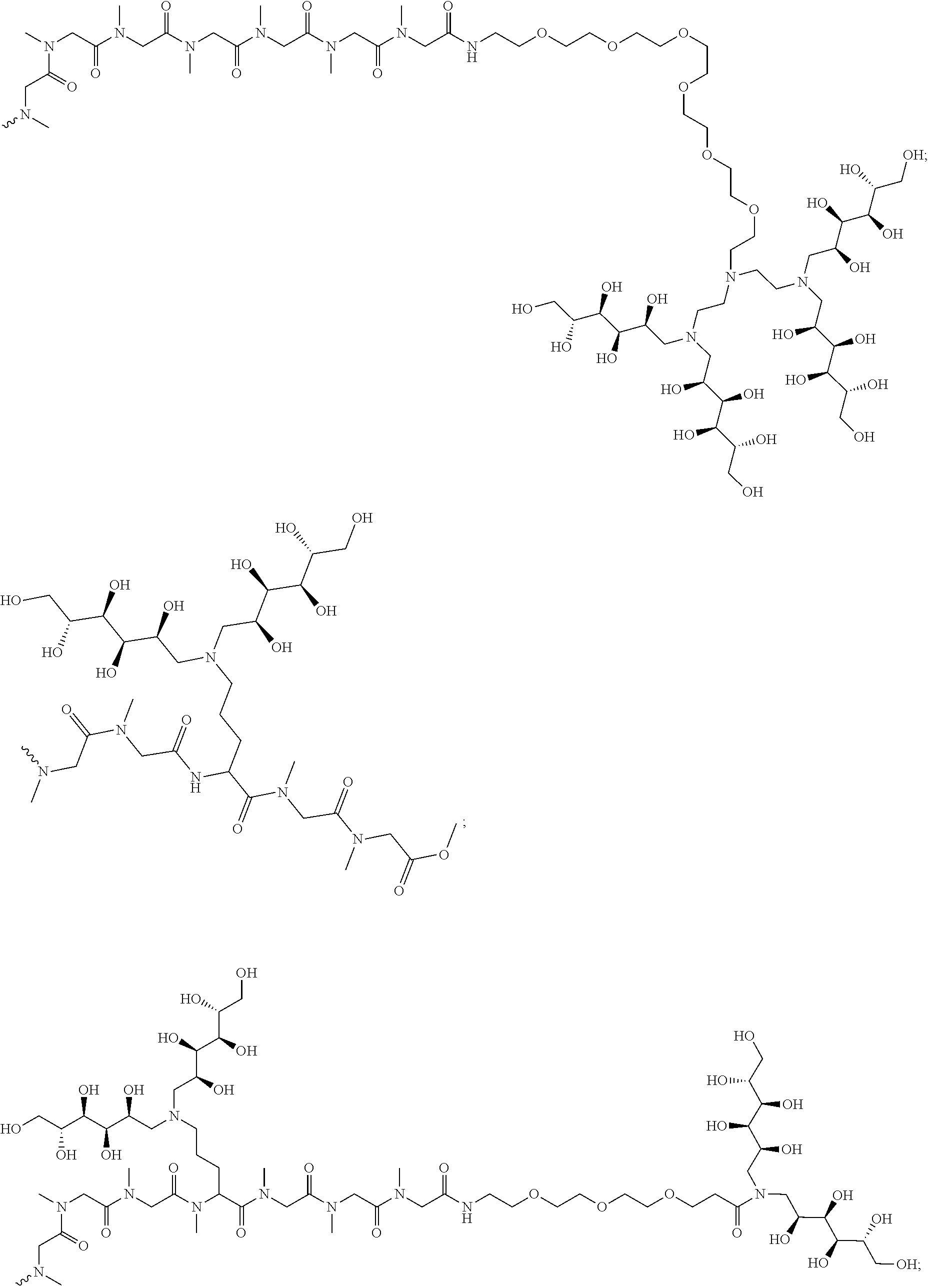 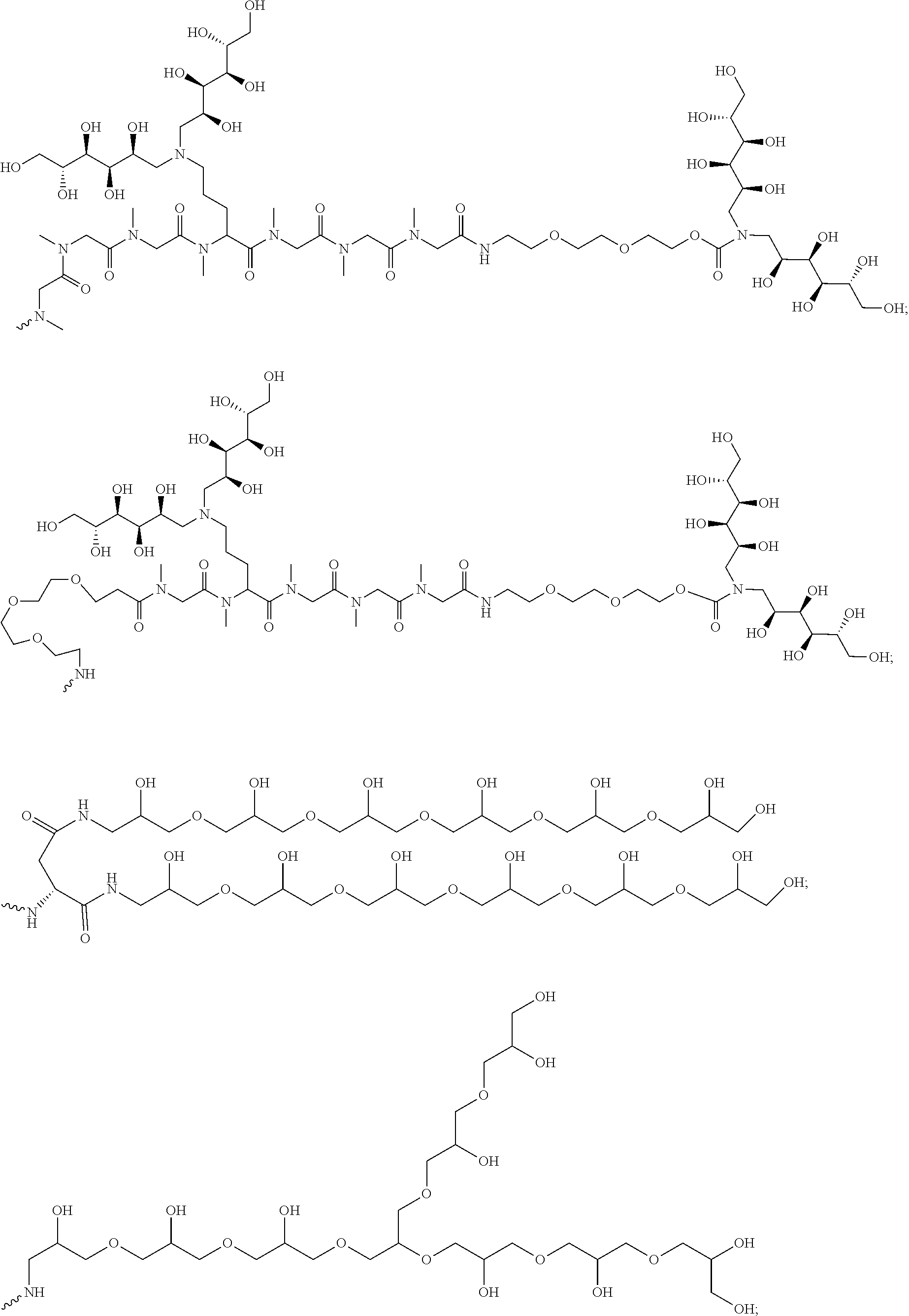 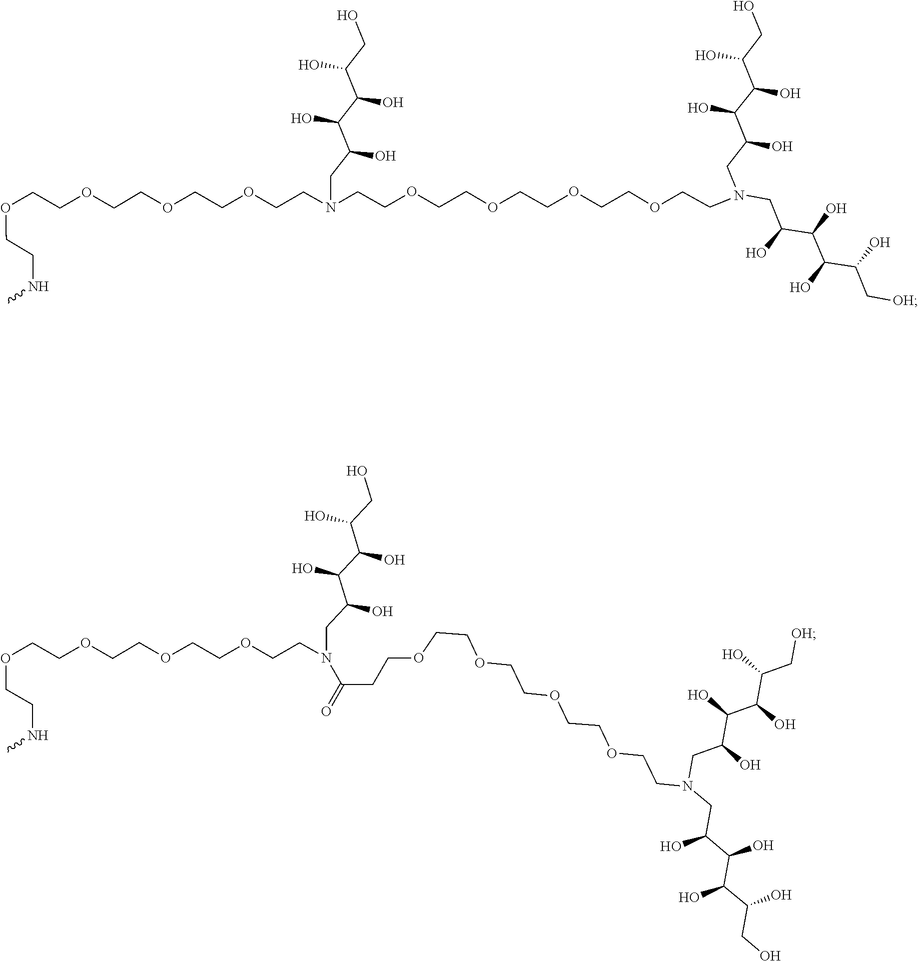 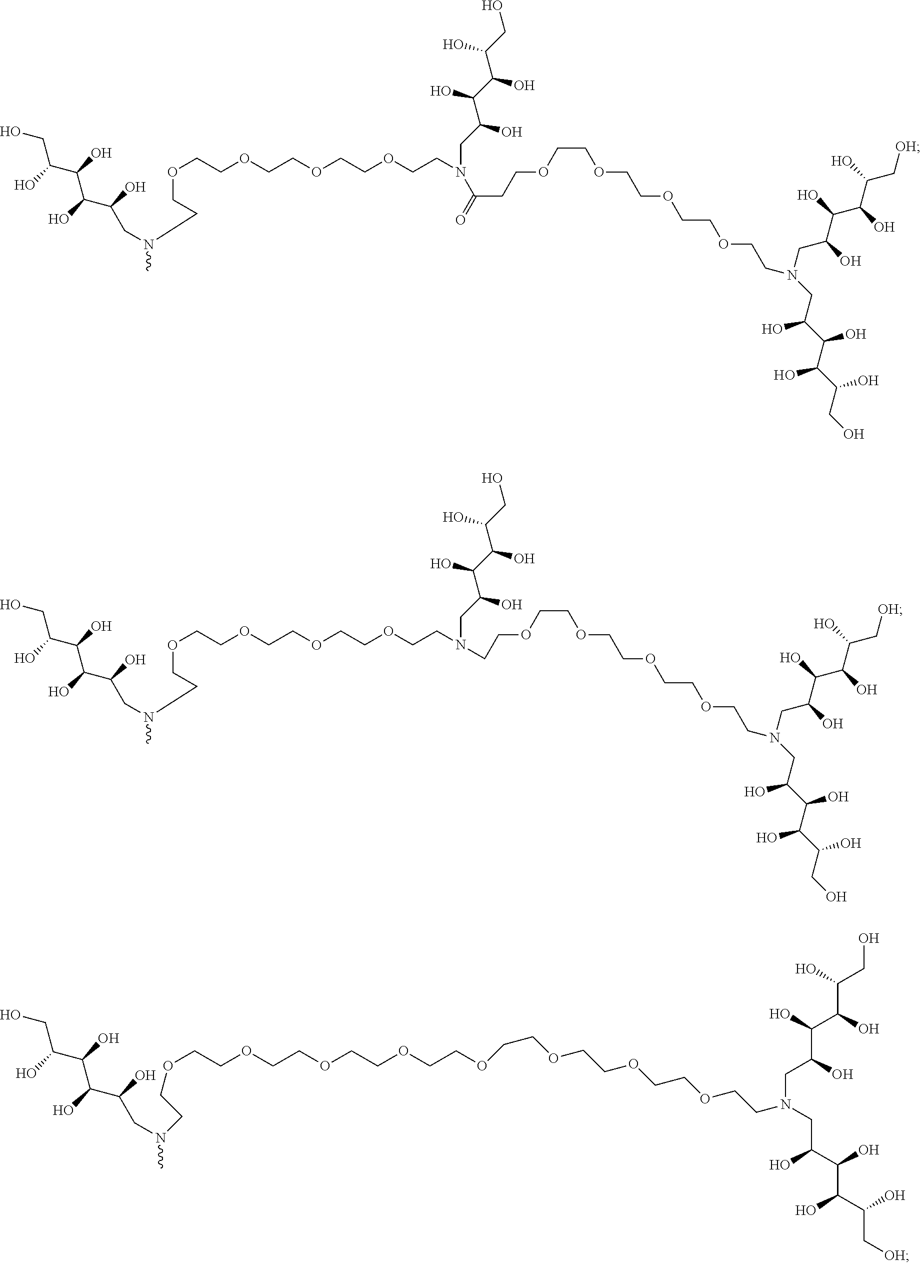 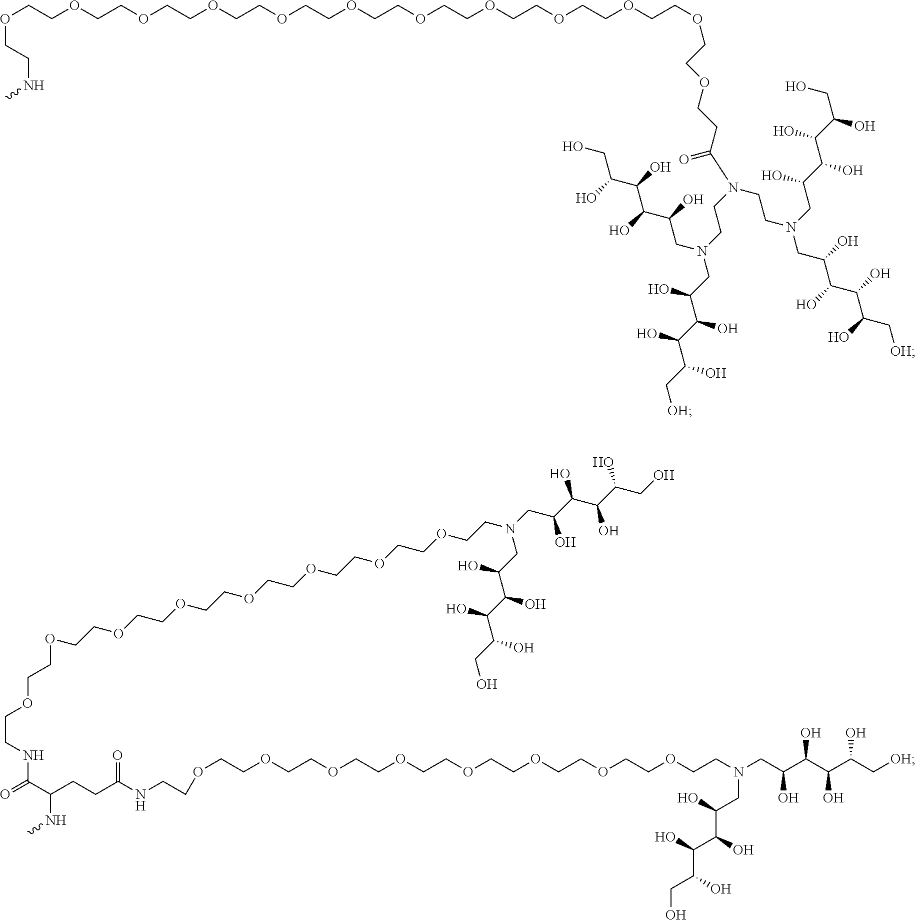 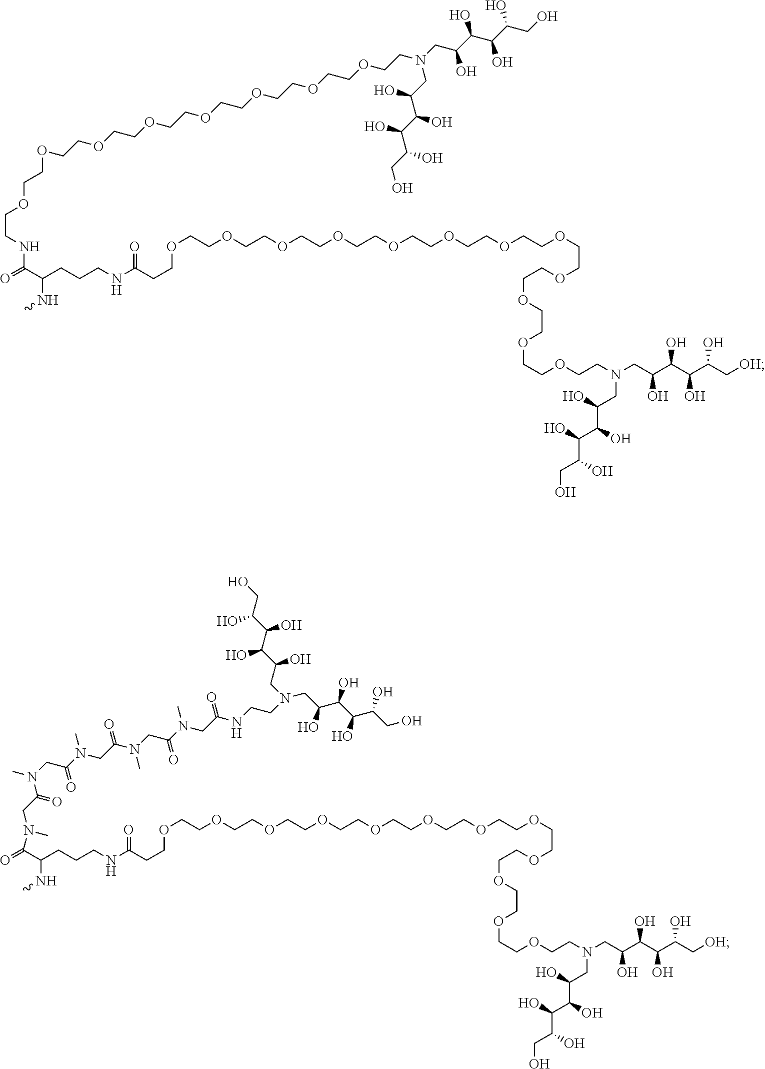 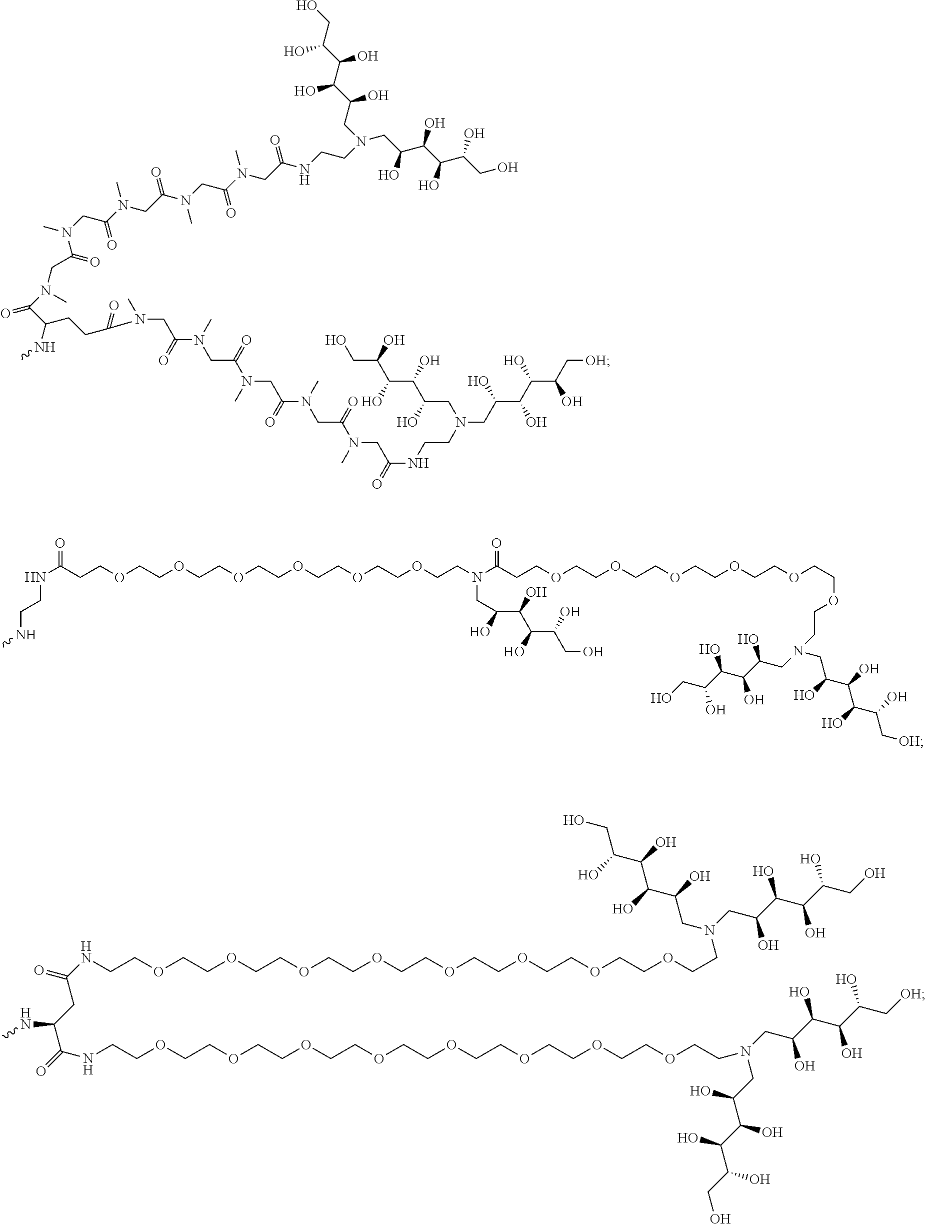 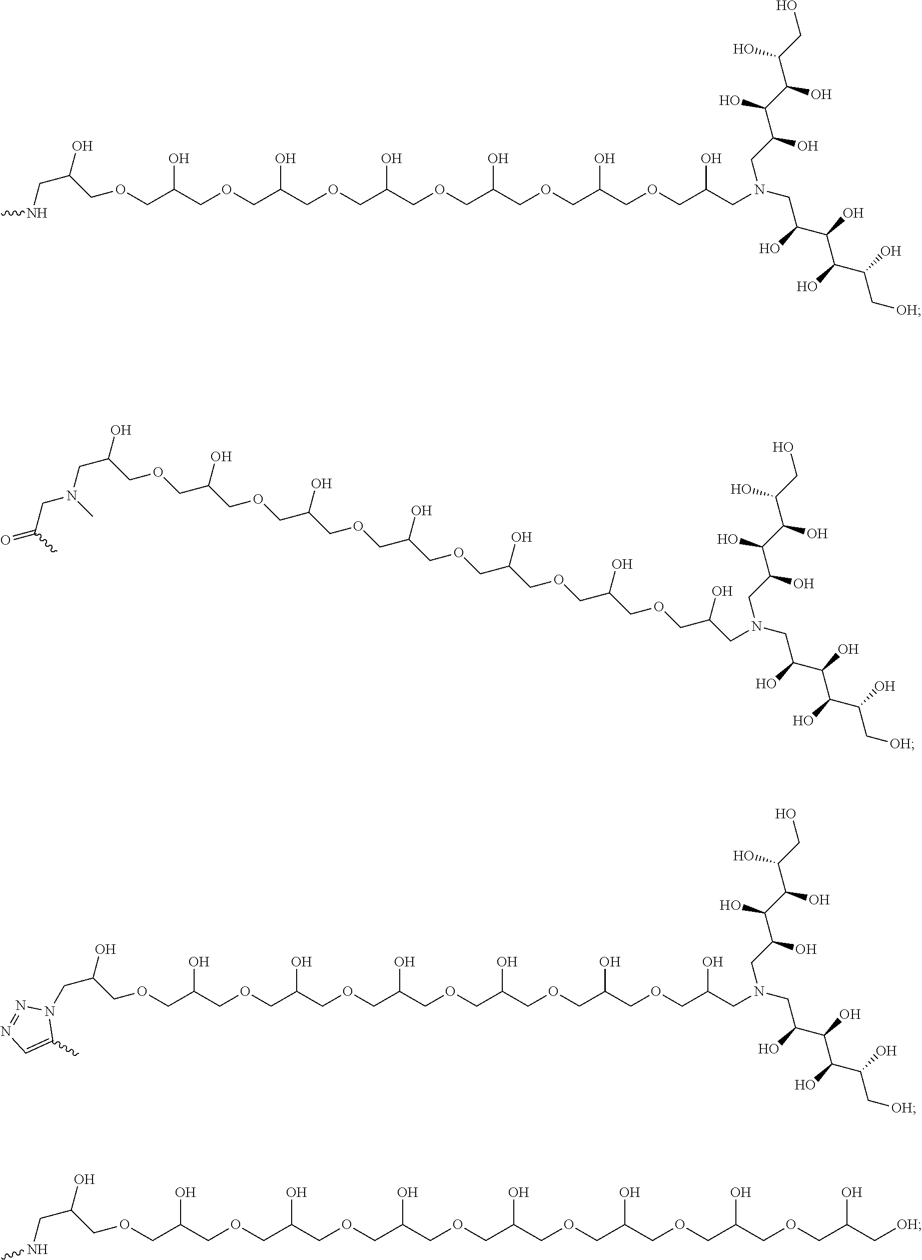 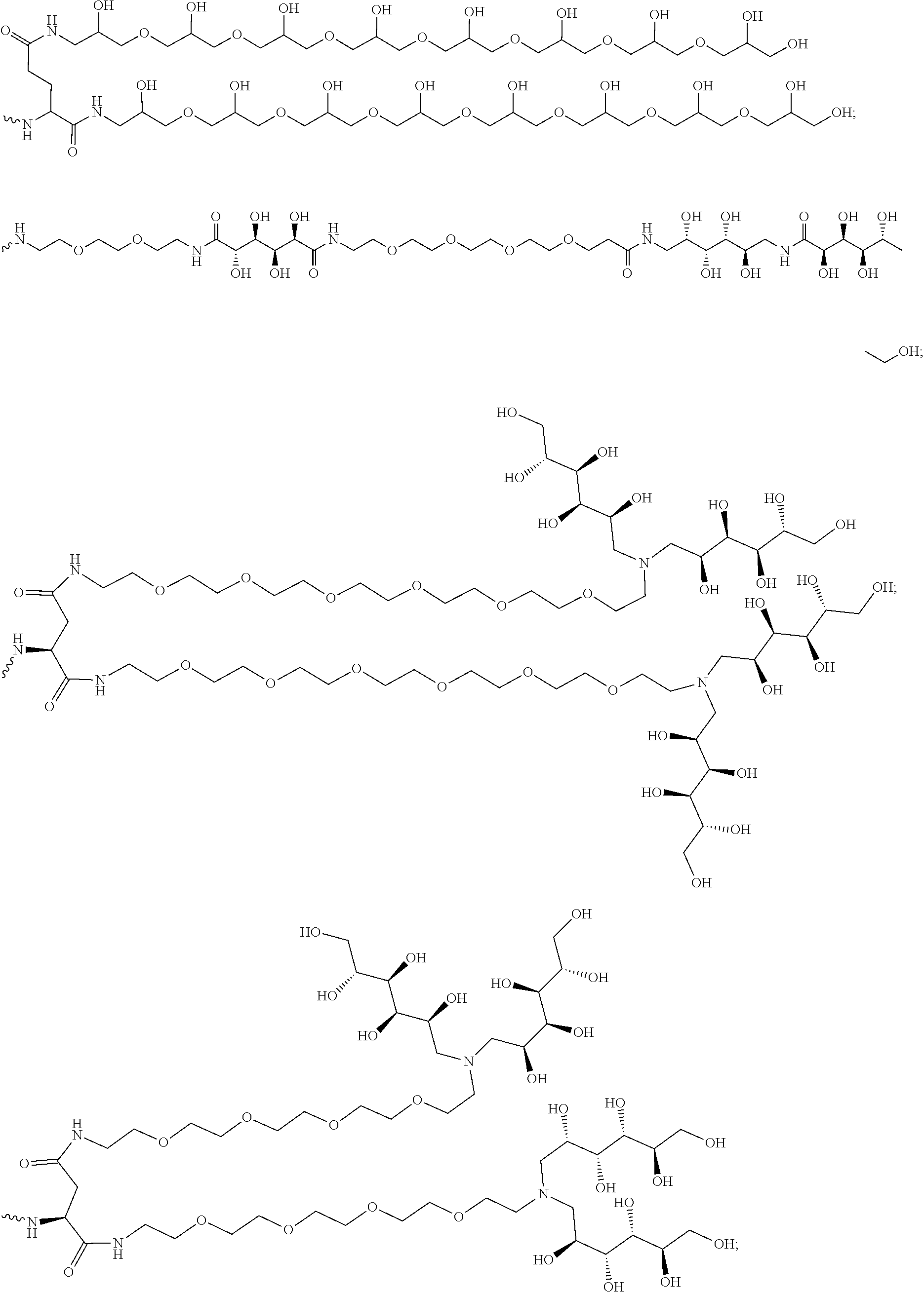 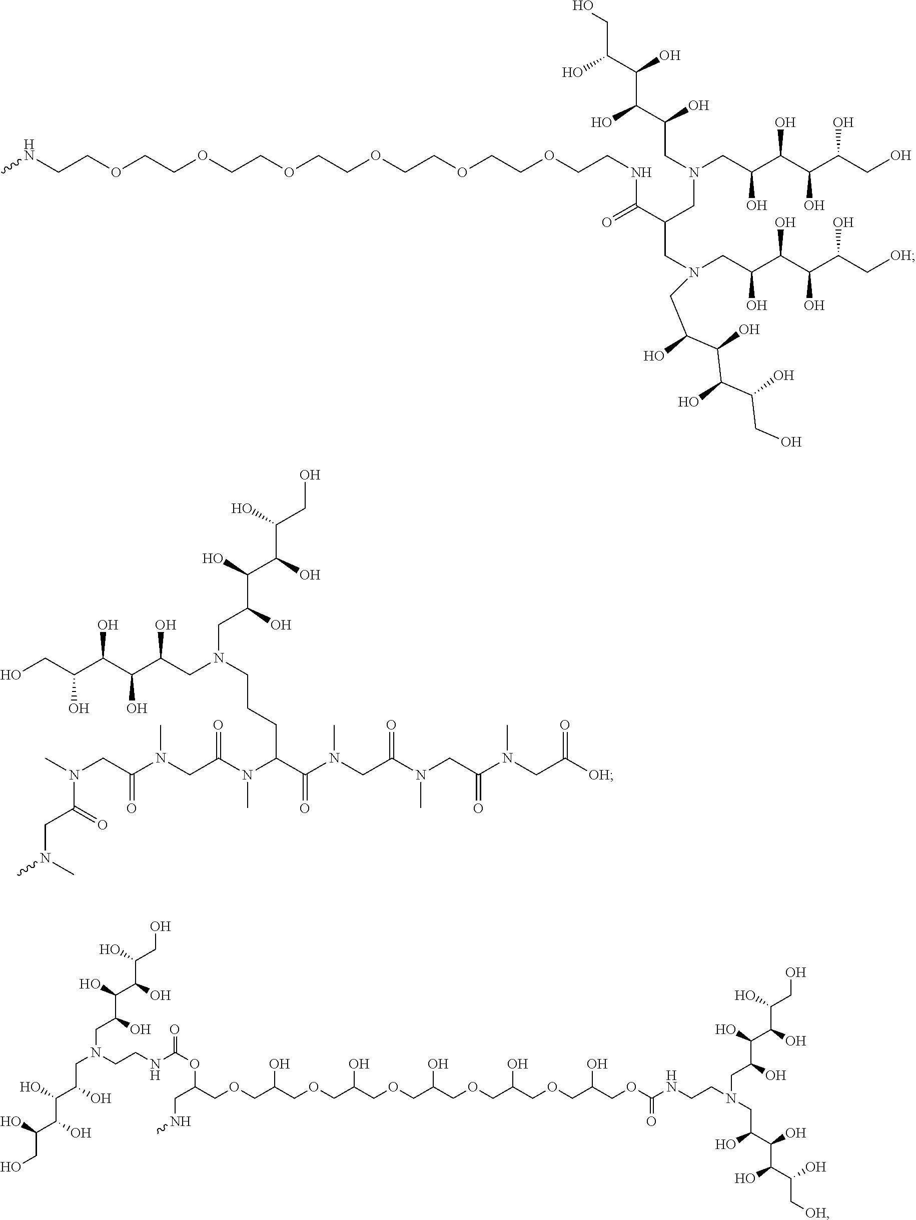 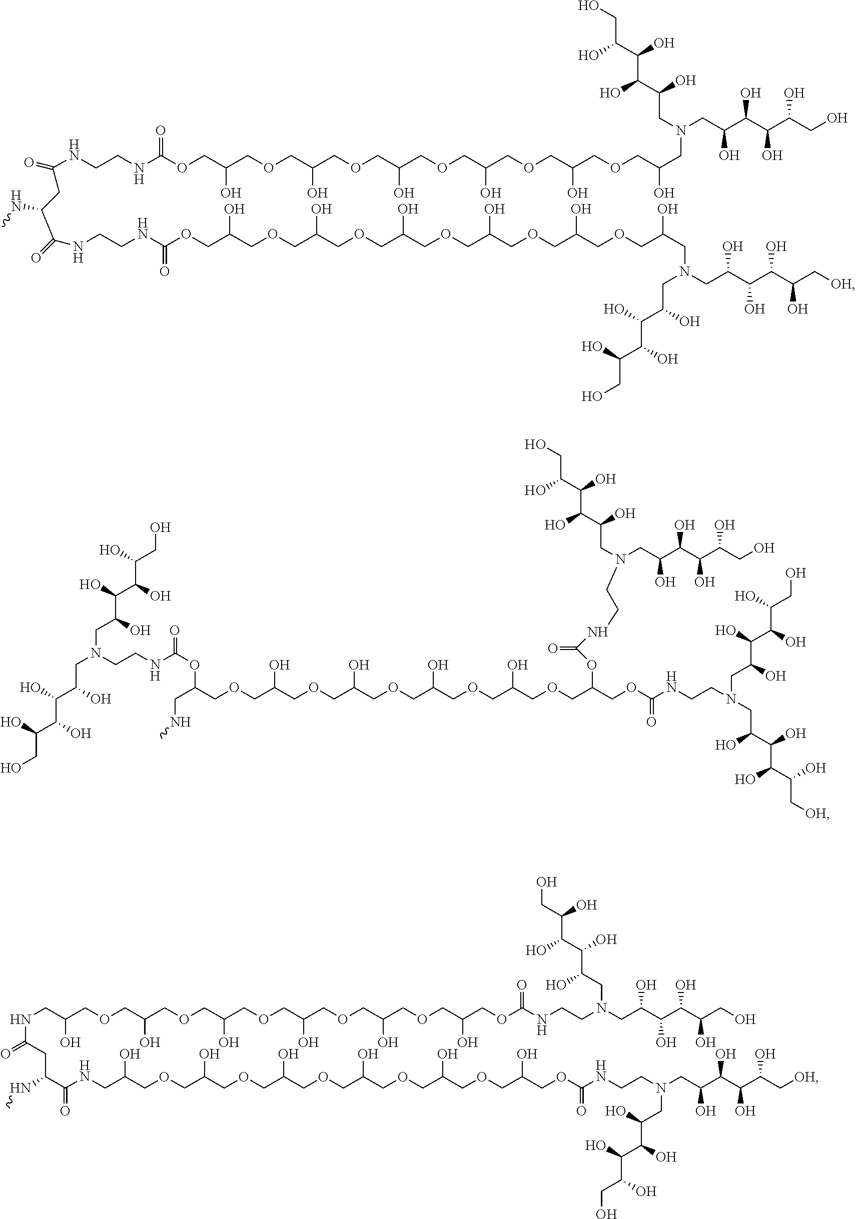 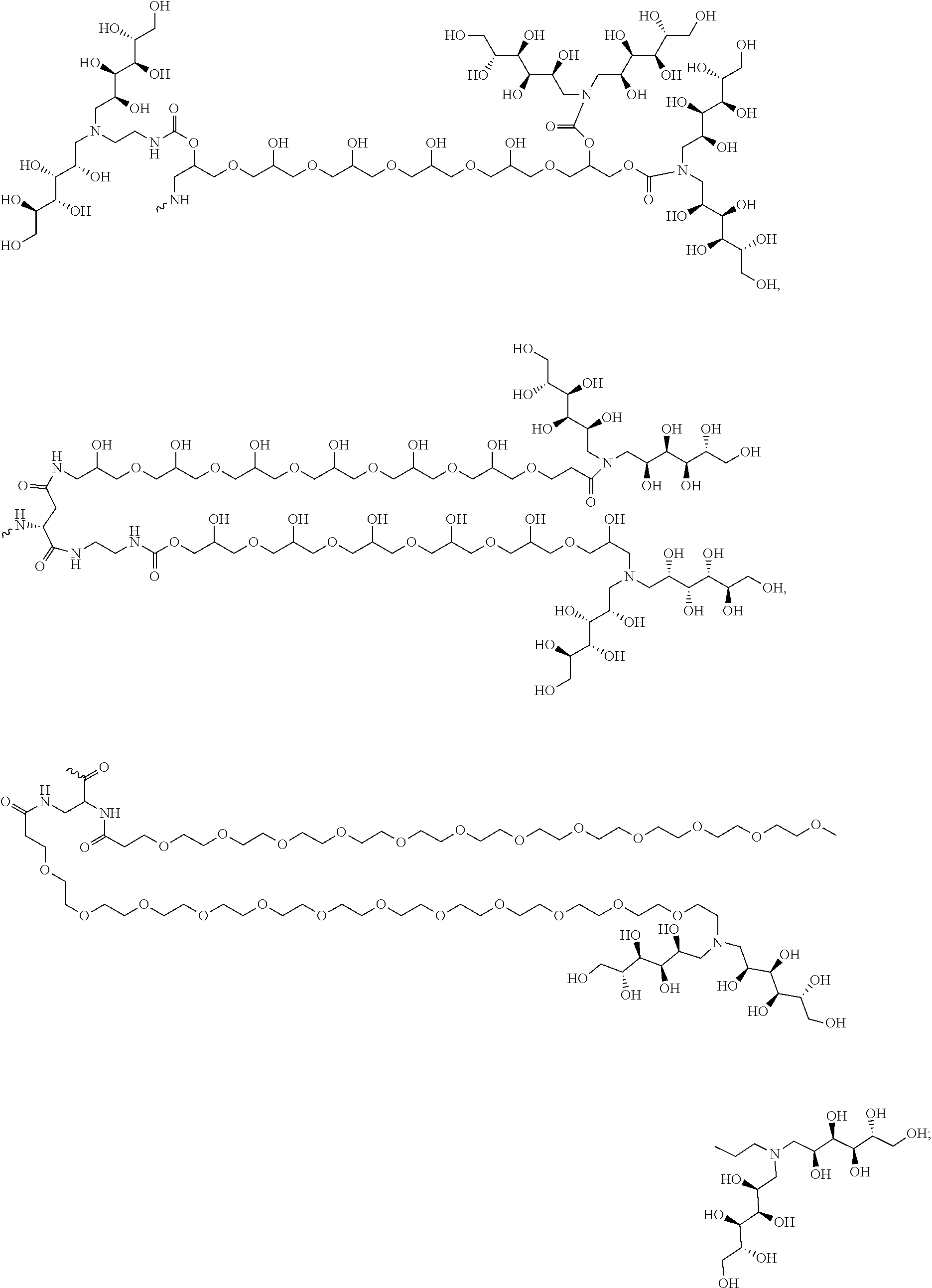 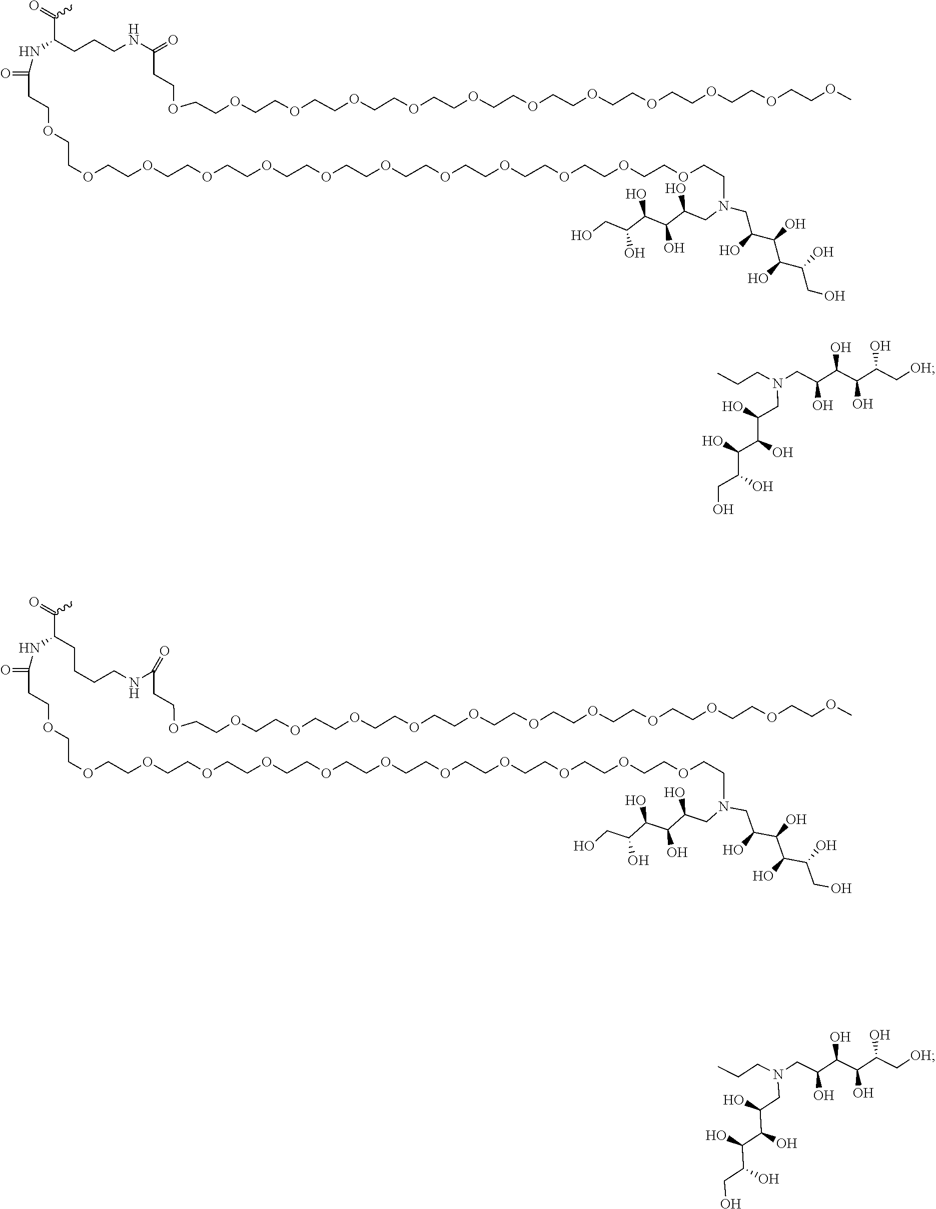 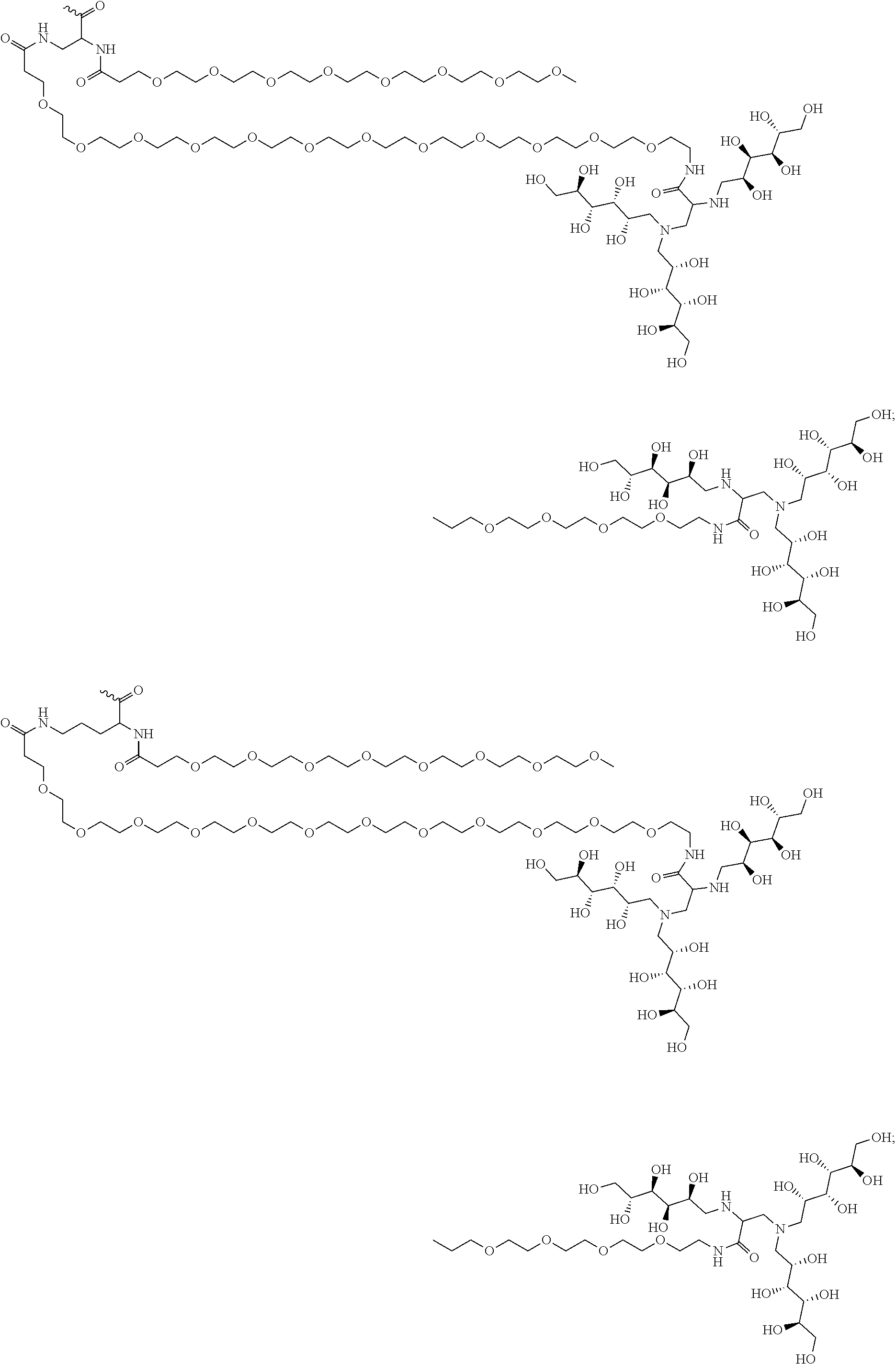 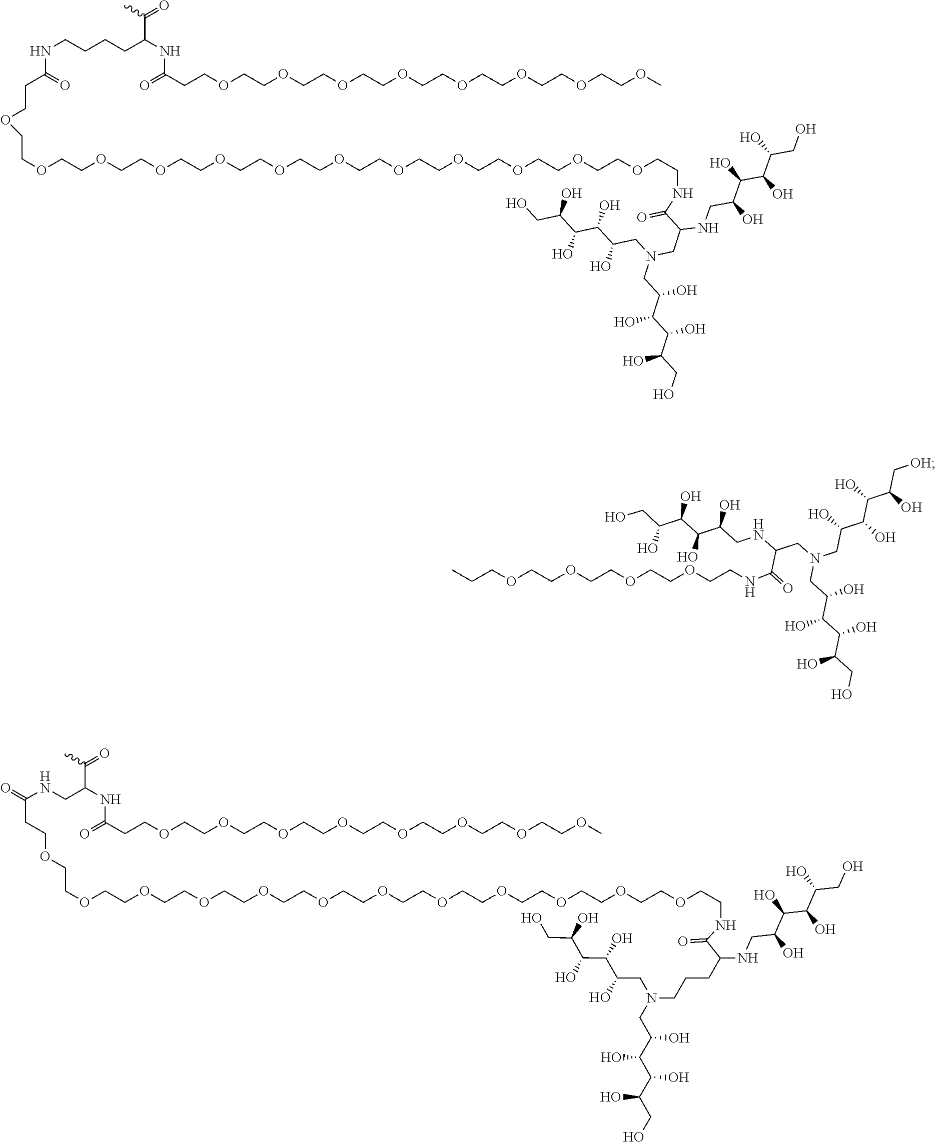 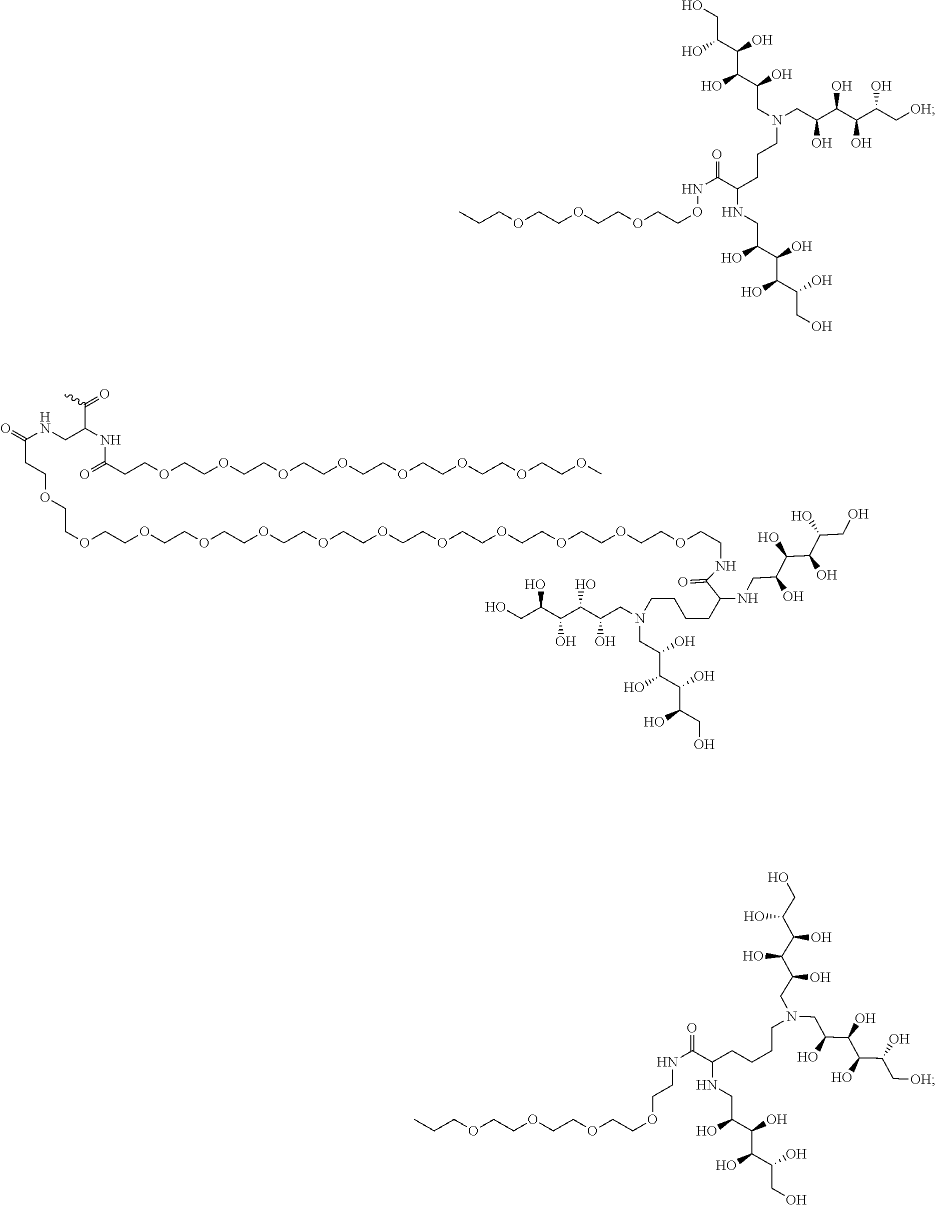 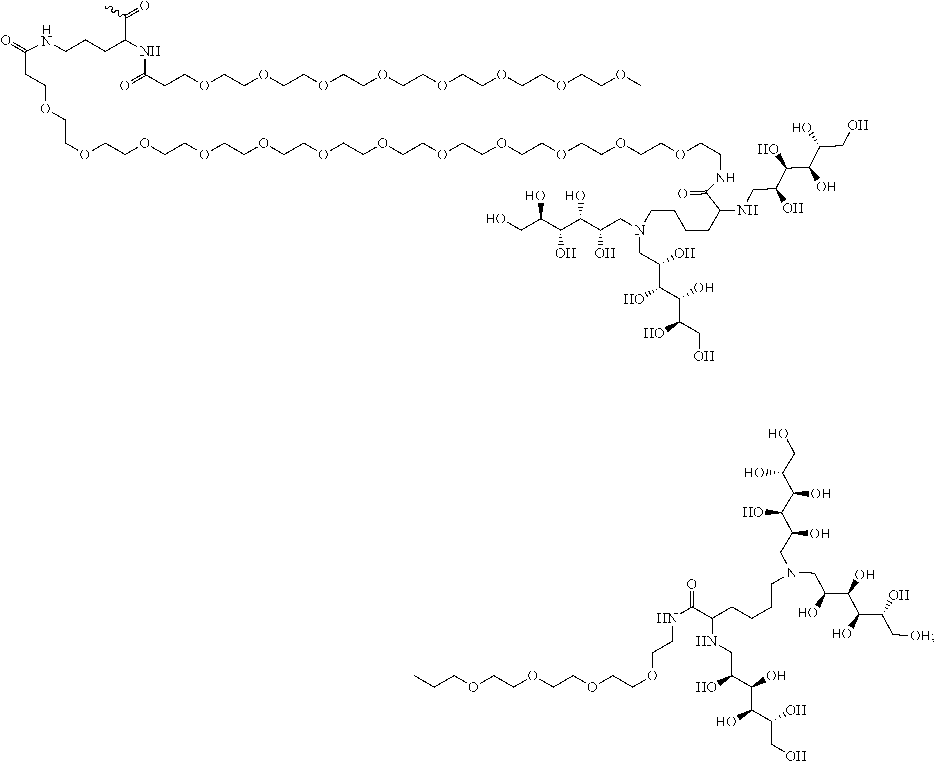 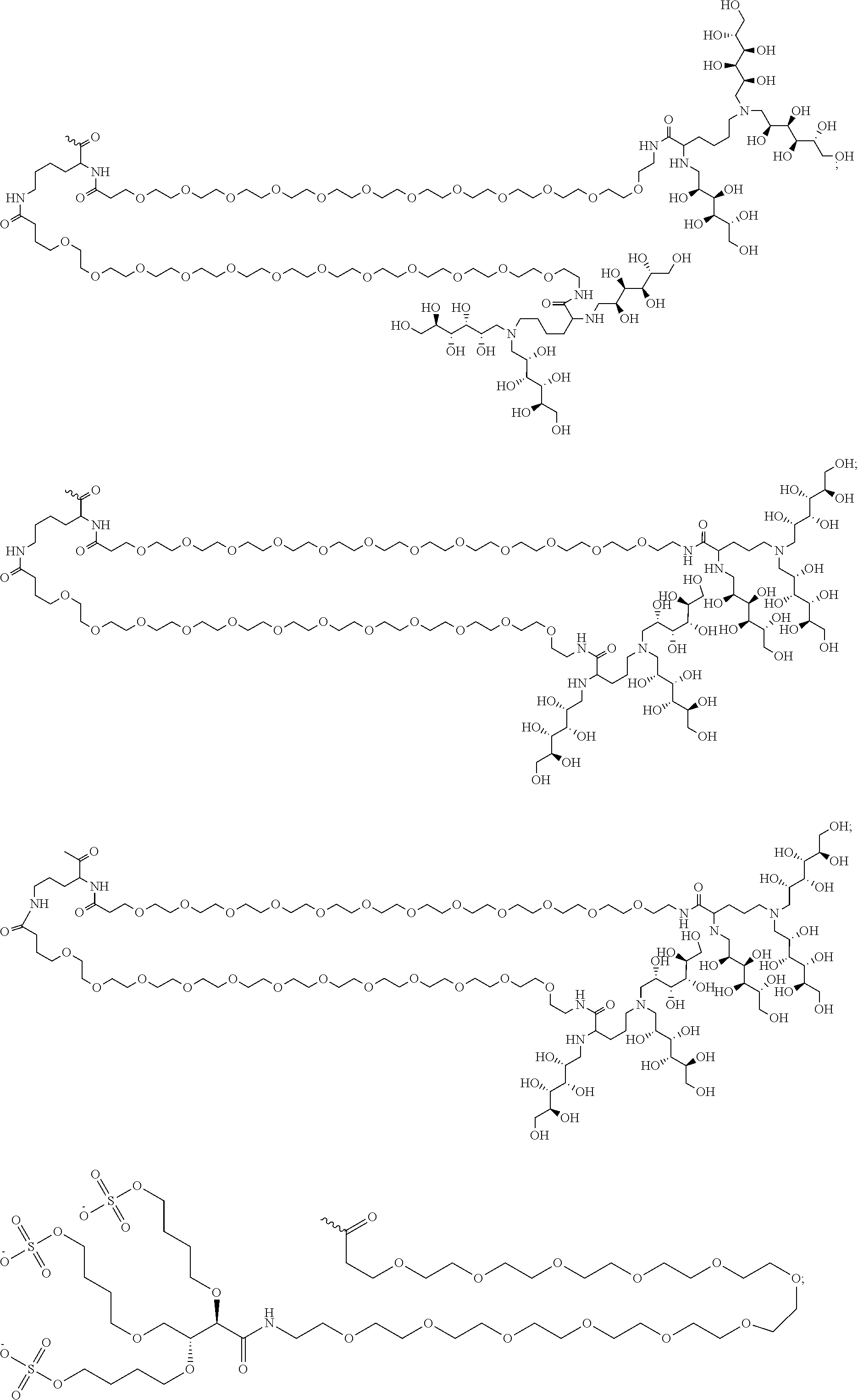 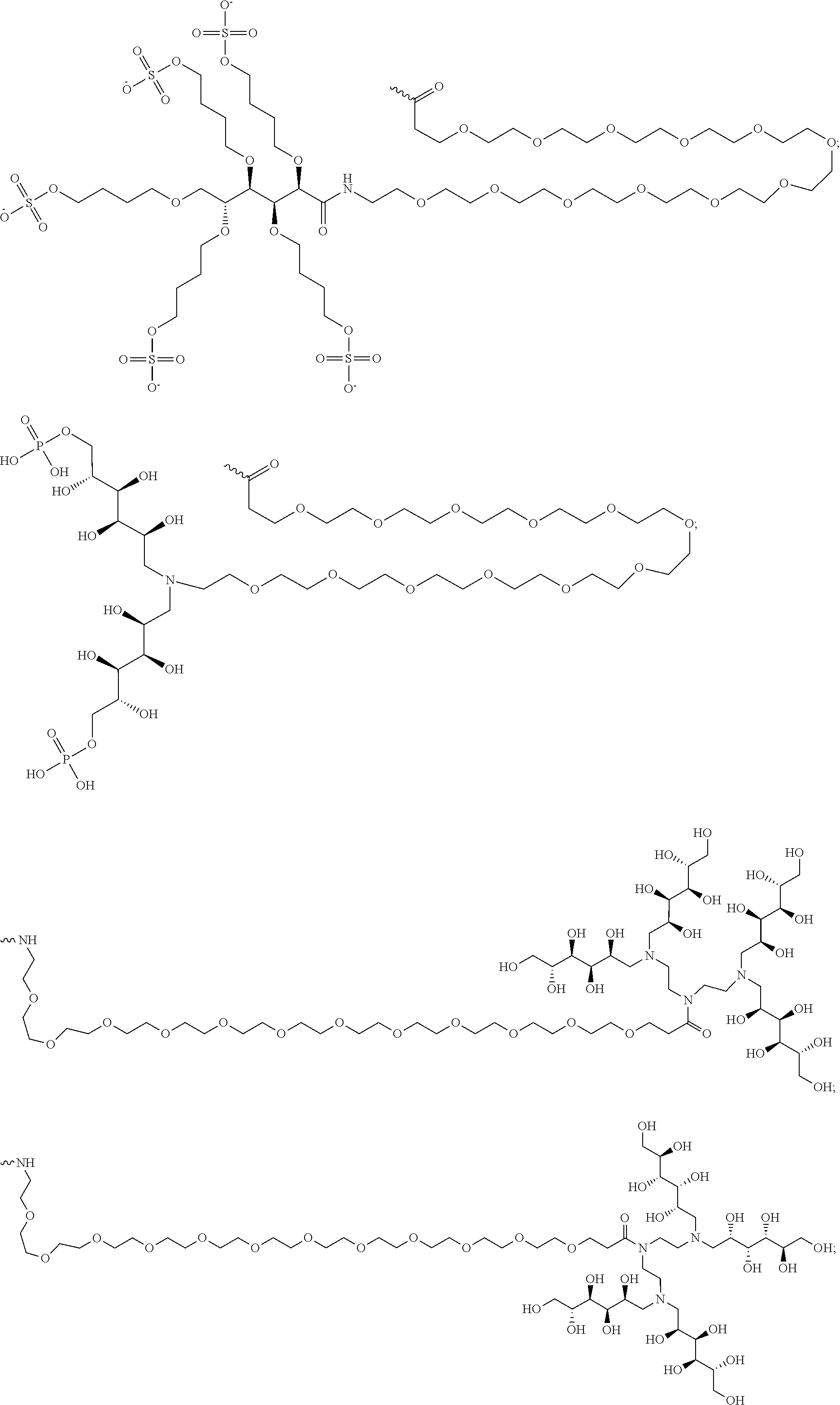 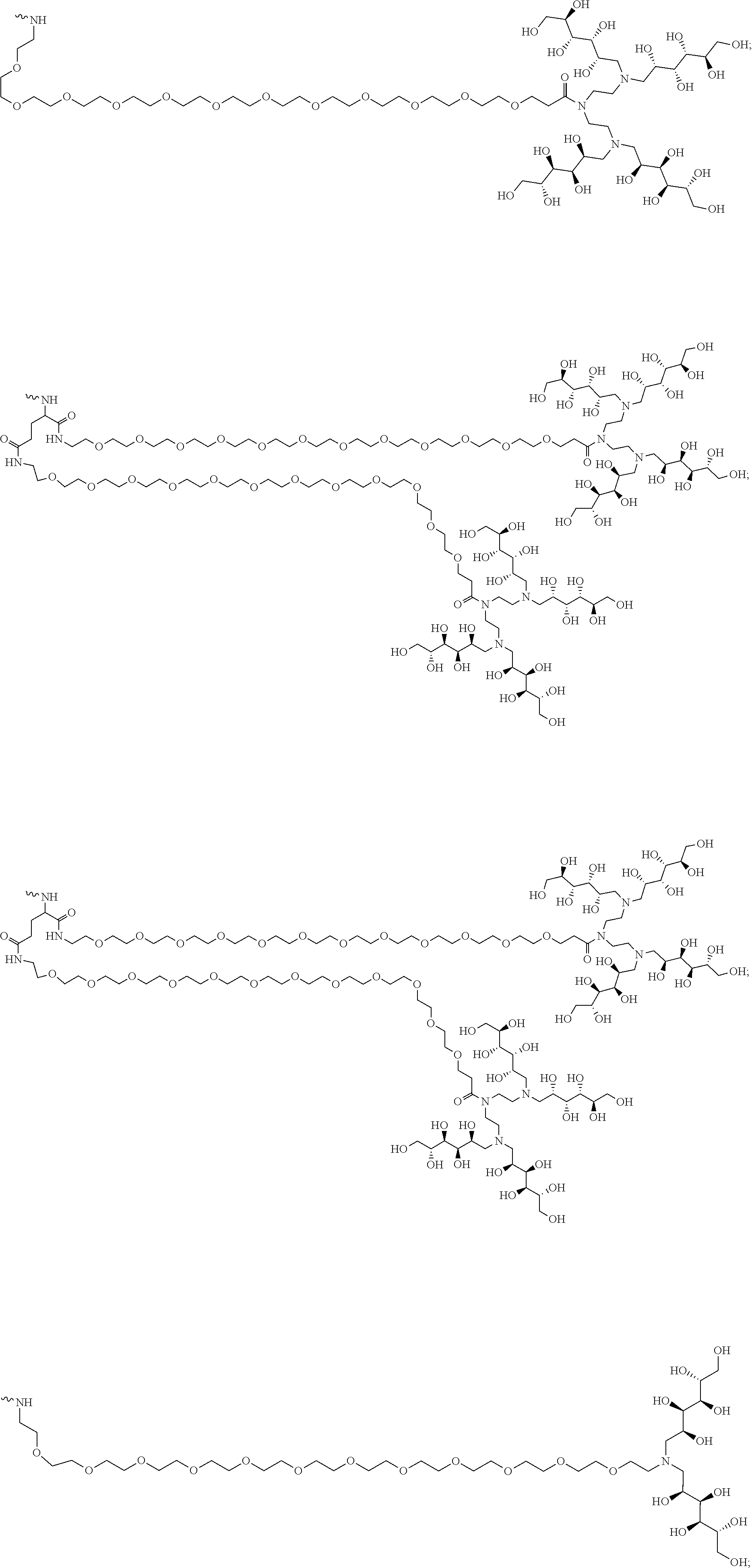 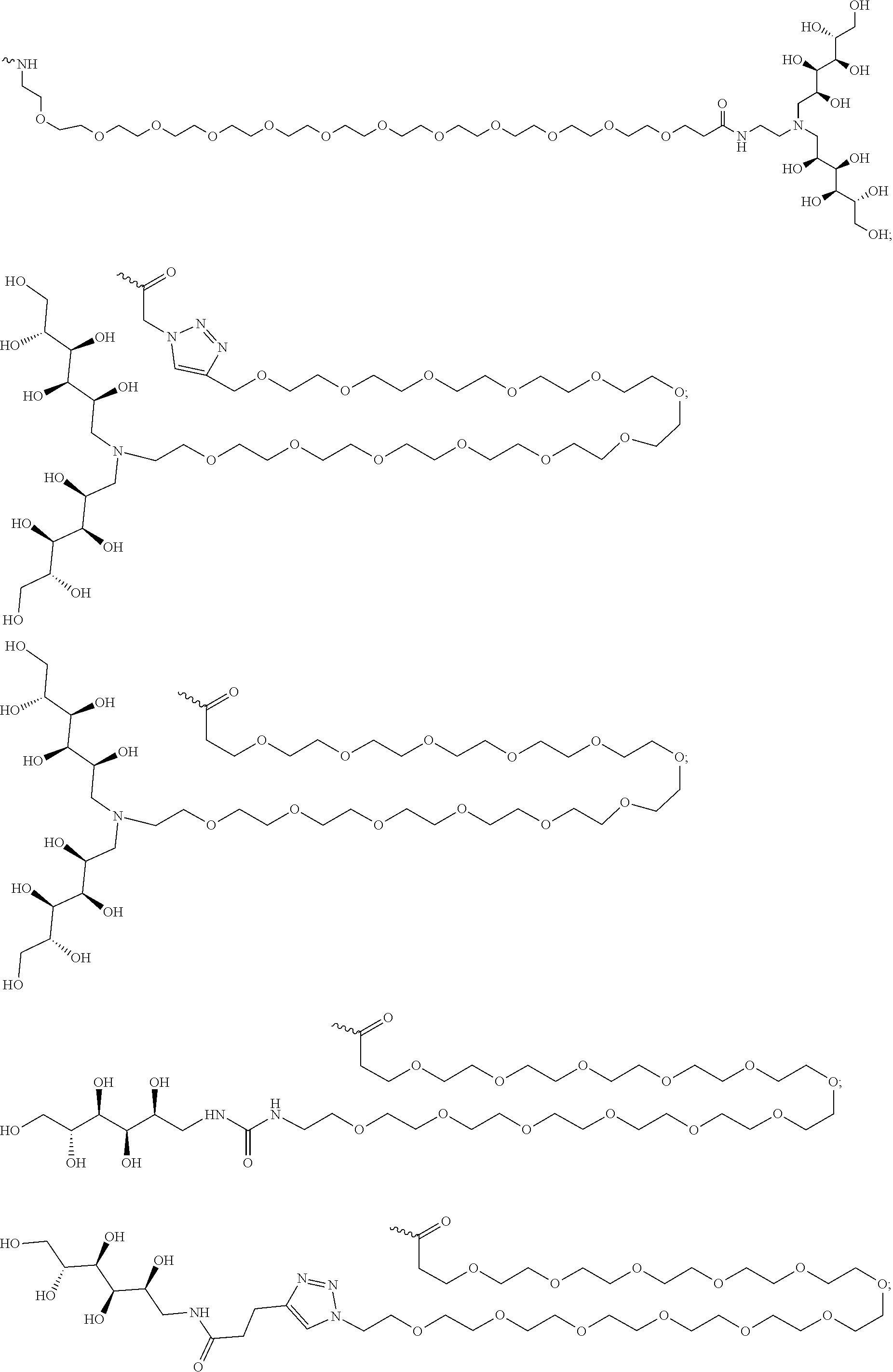 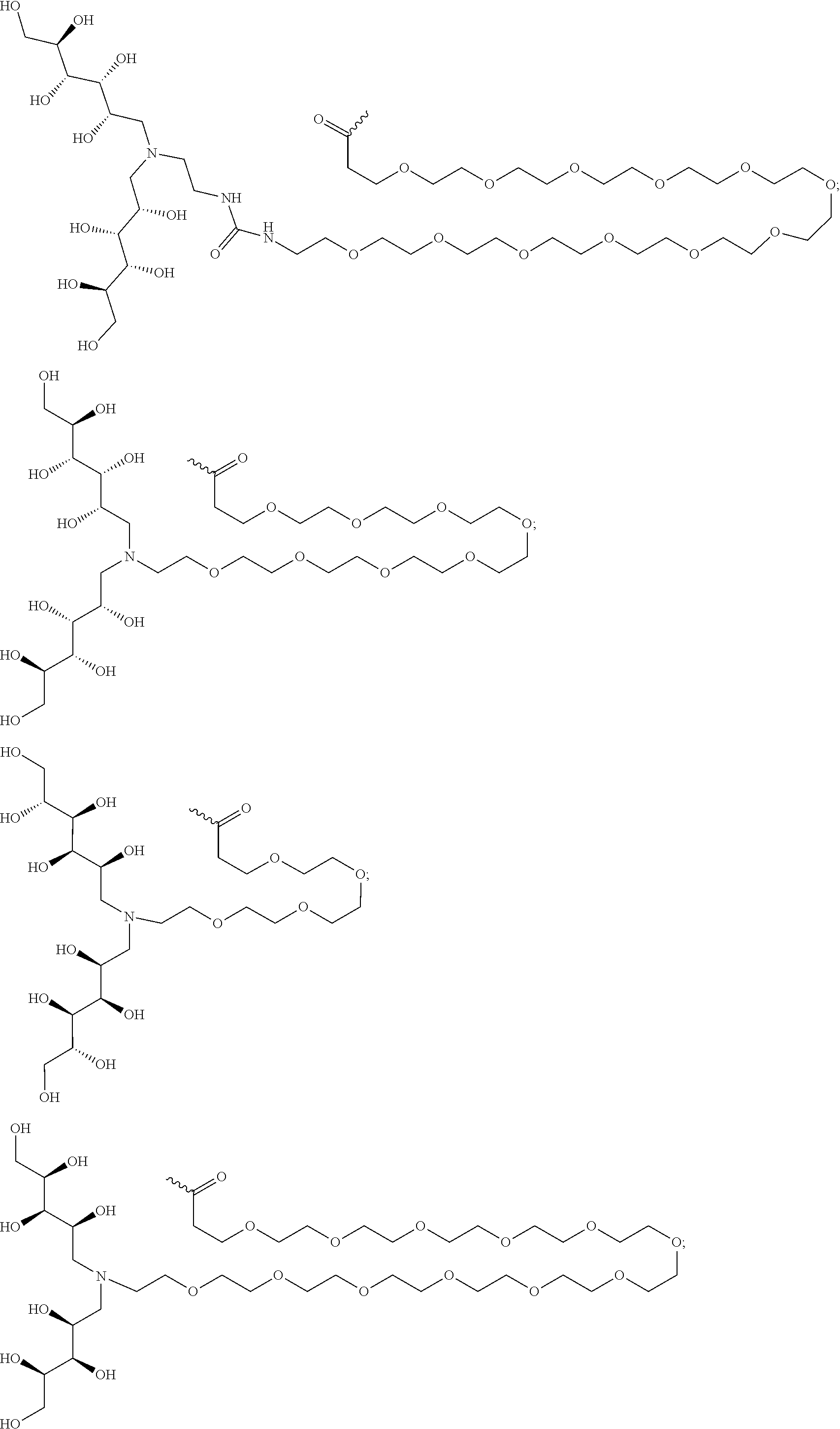 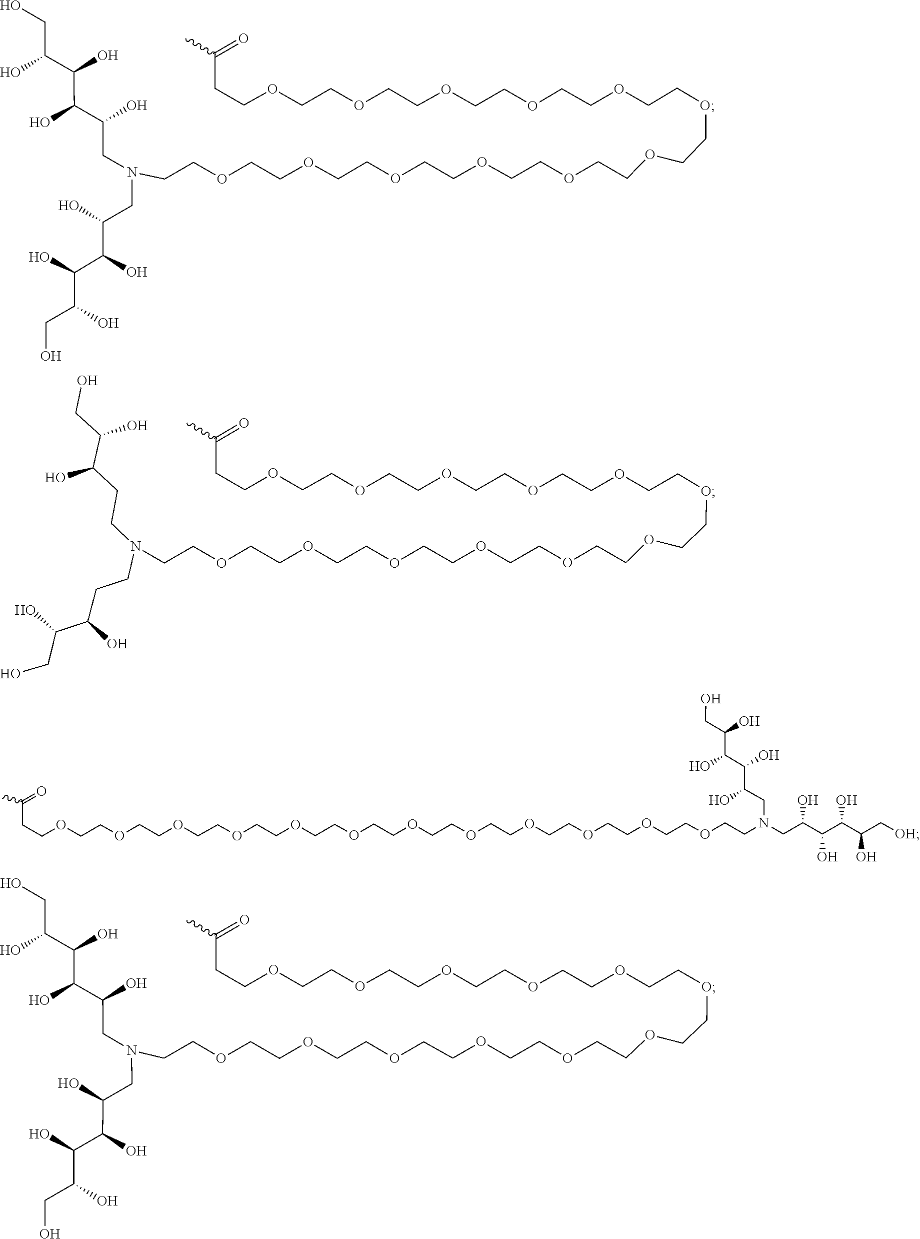 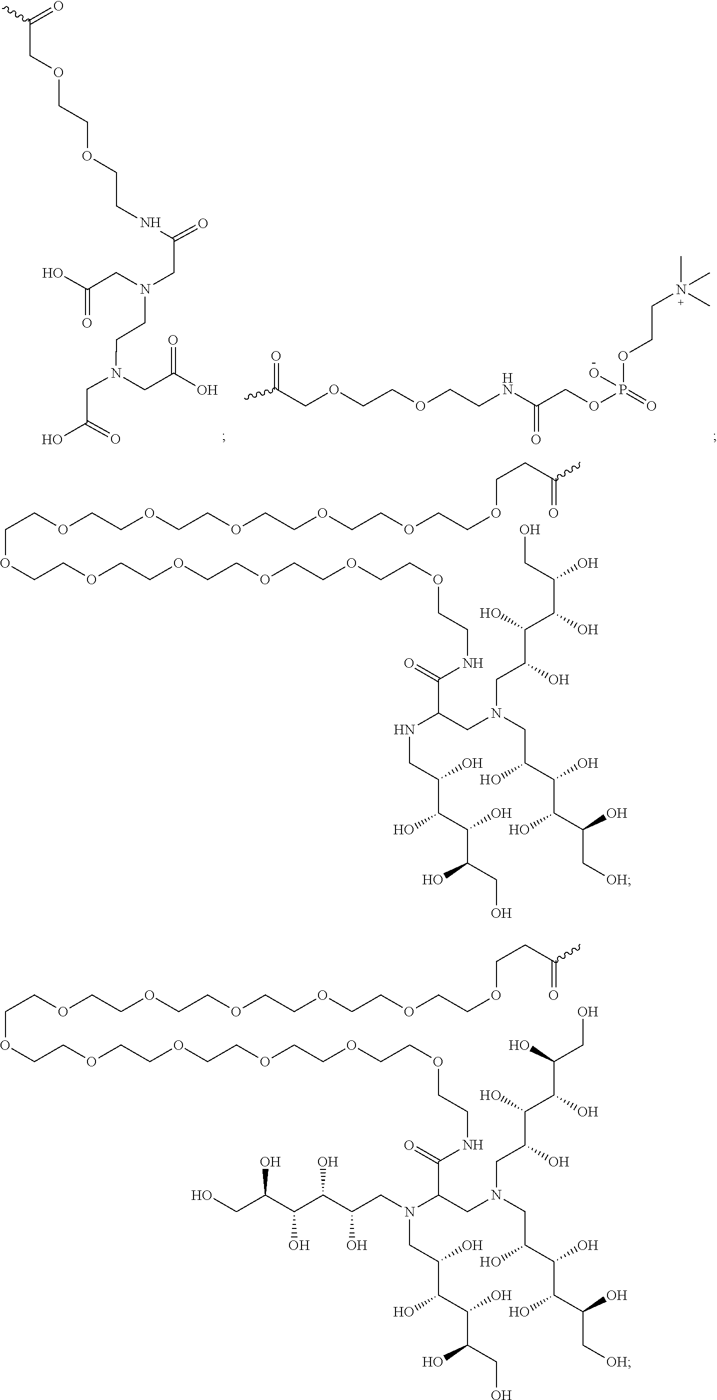 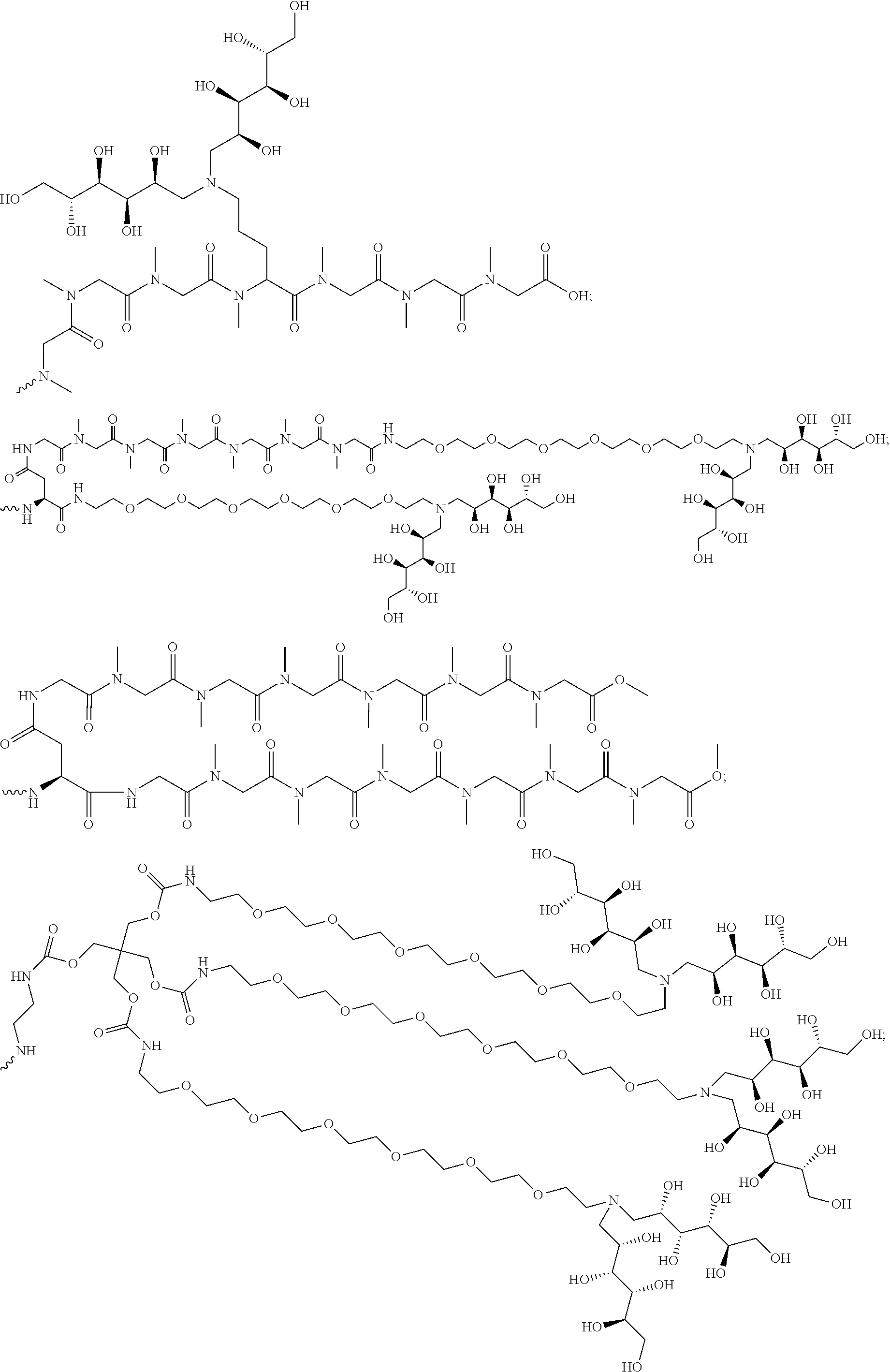 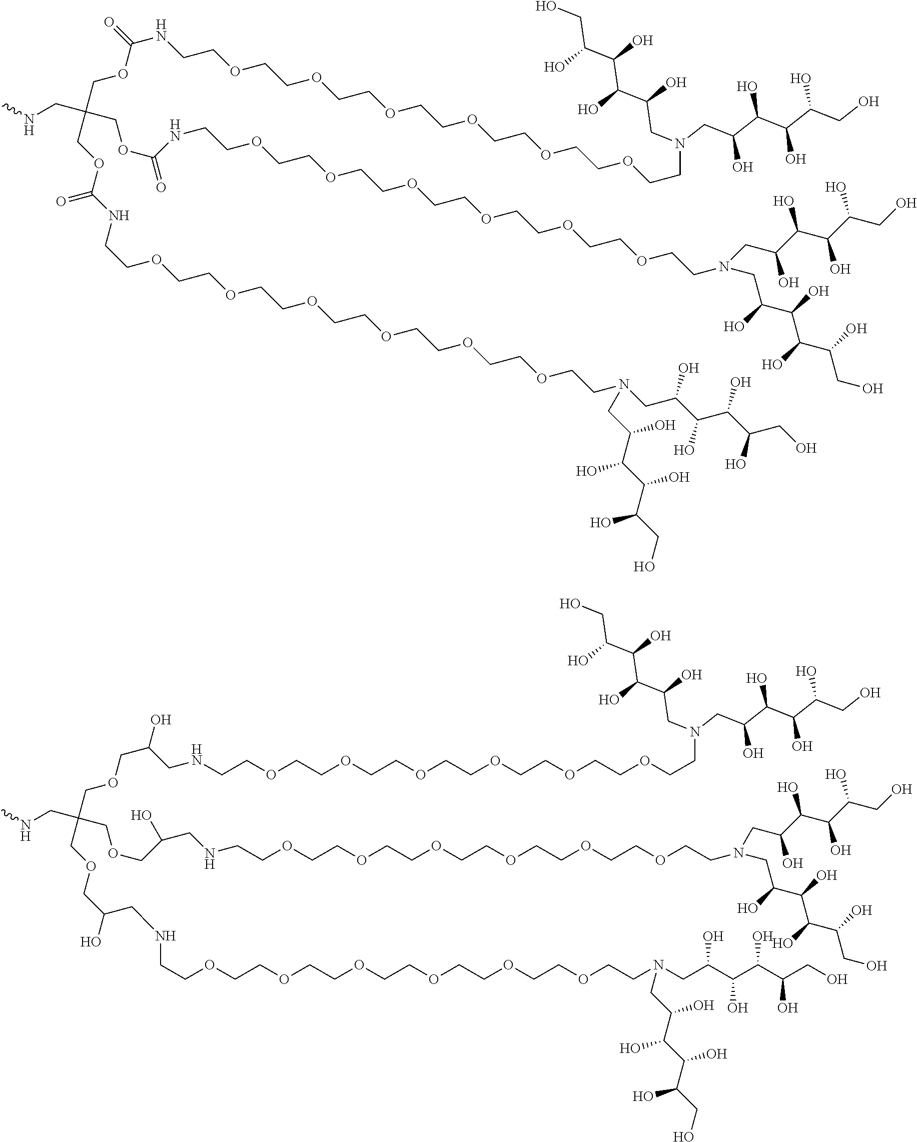 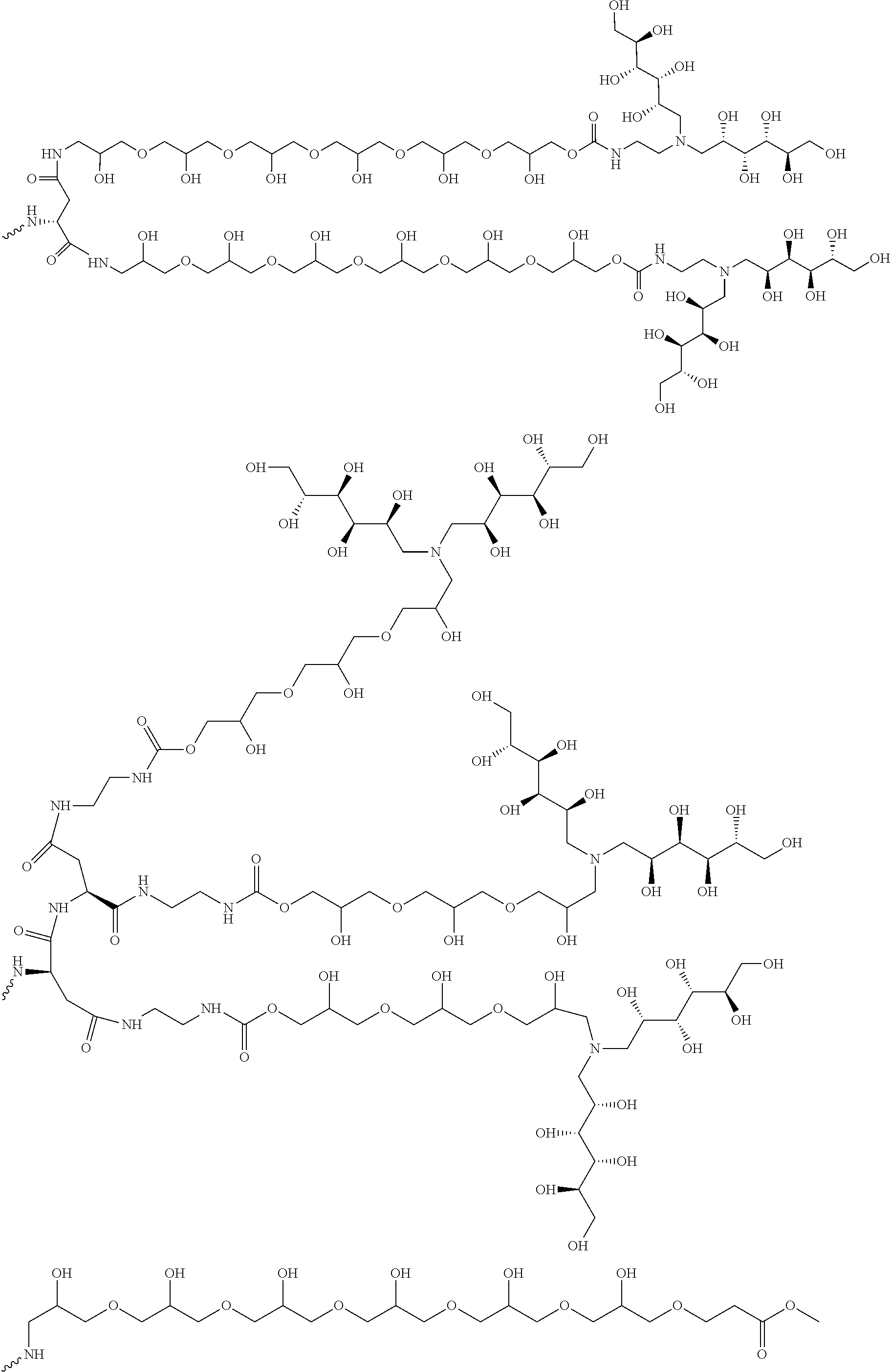 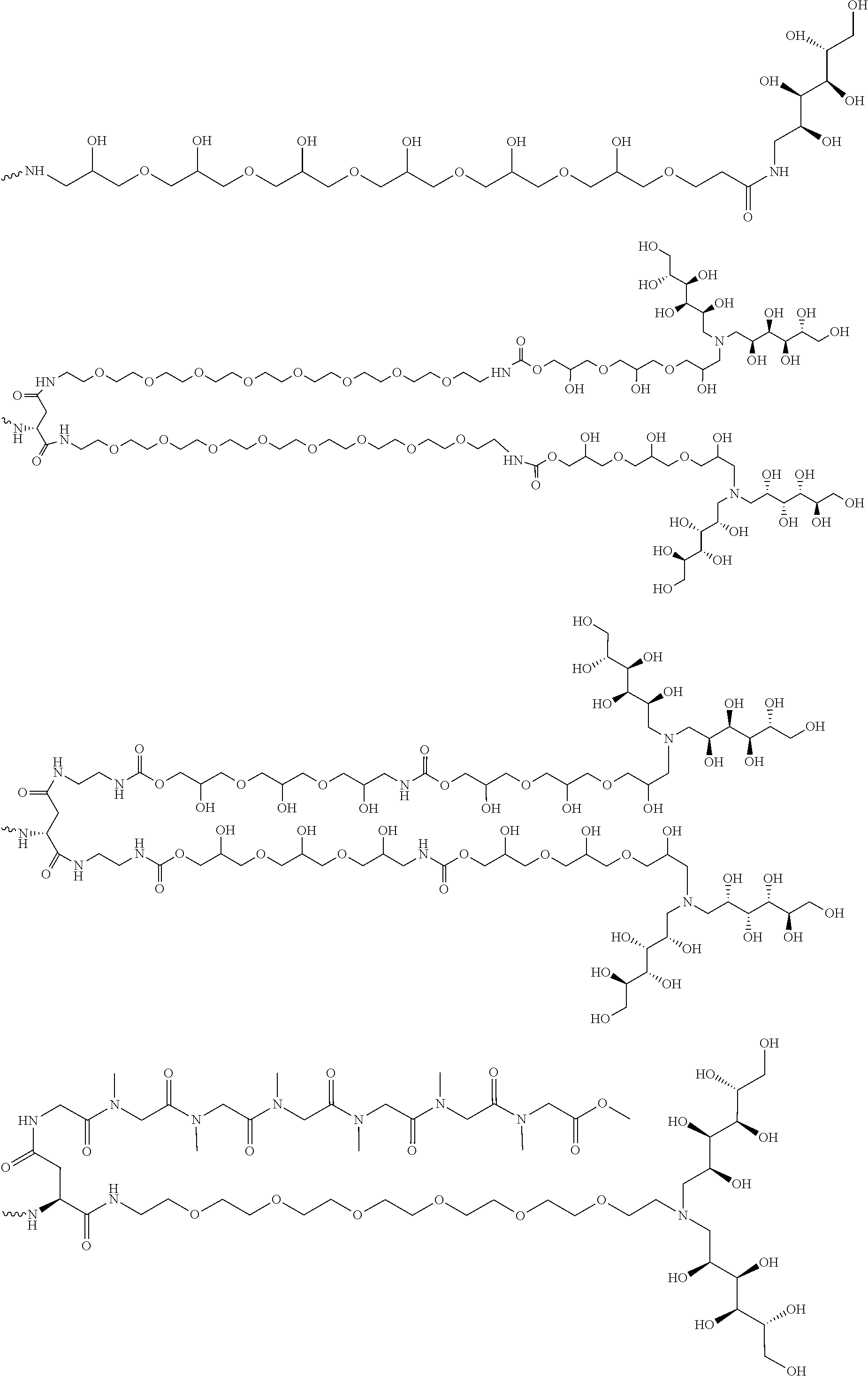 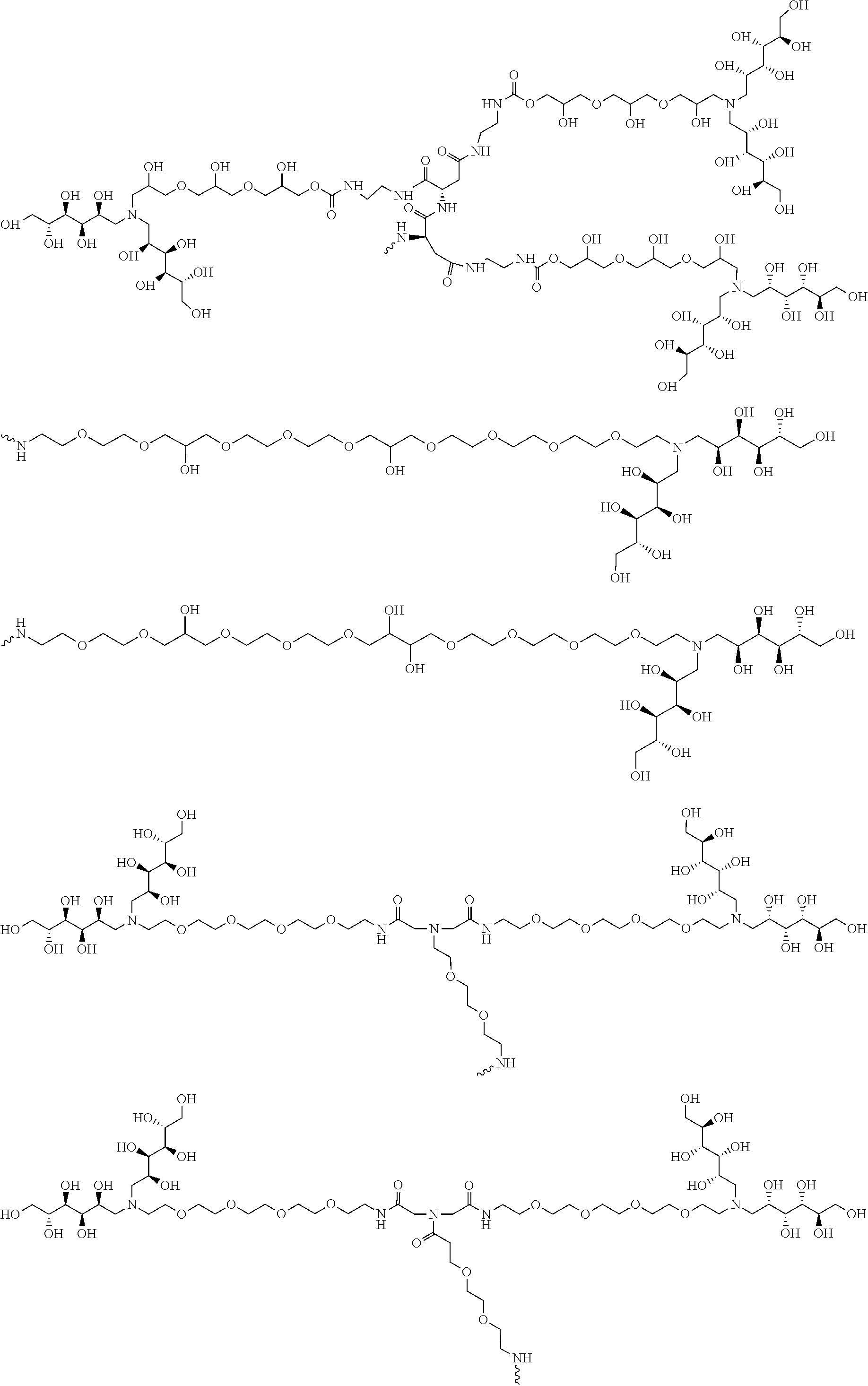 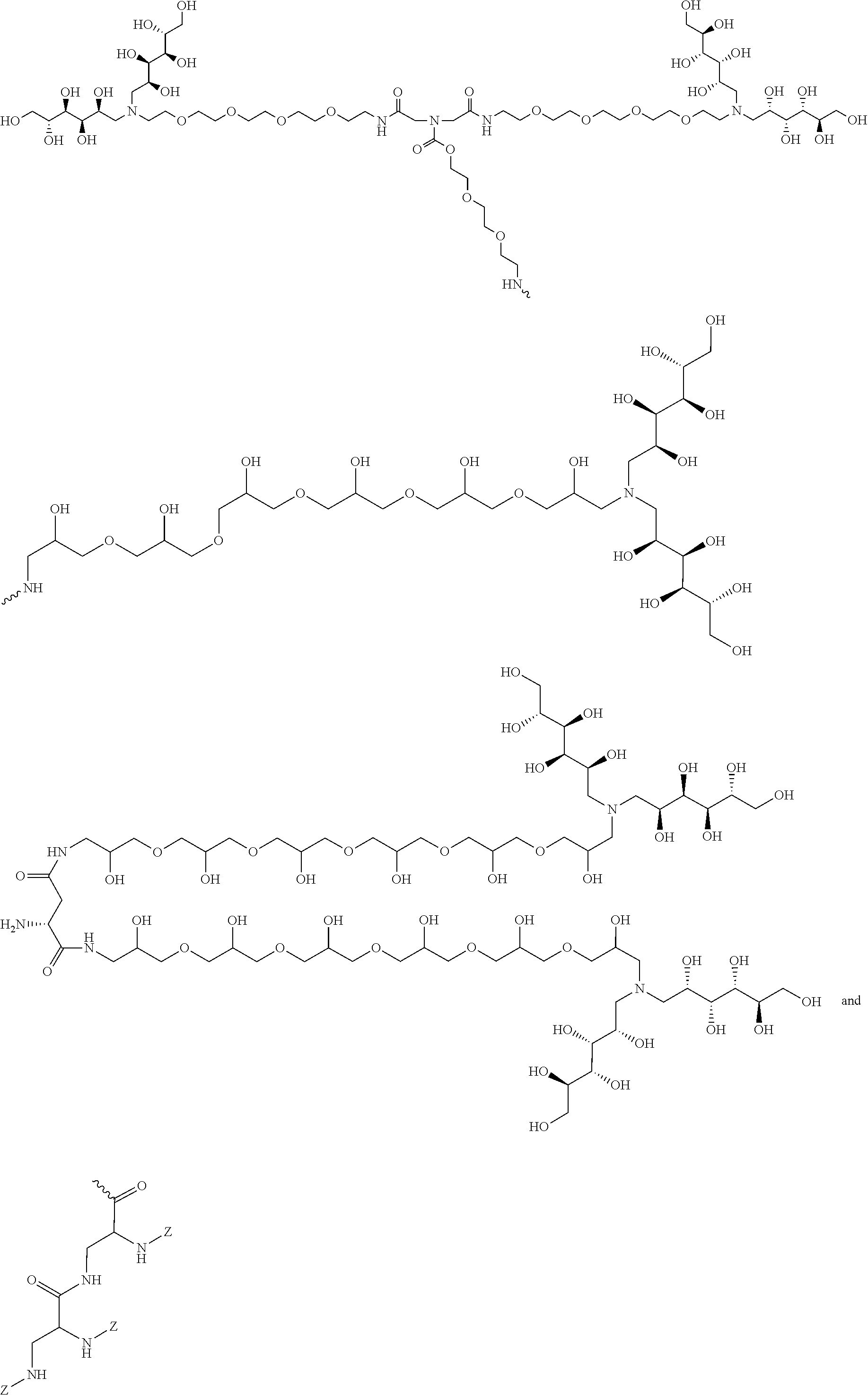
wherein each Z is attached at * and is individually selected from: 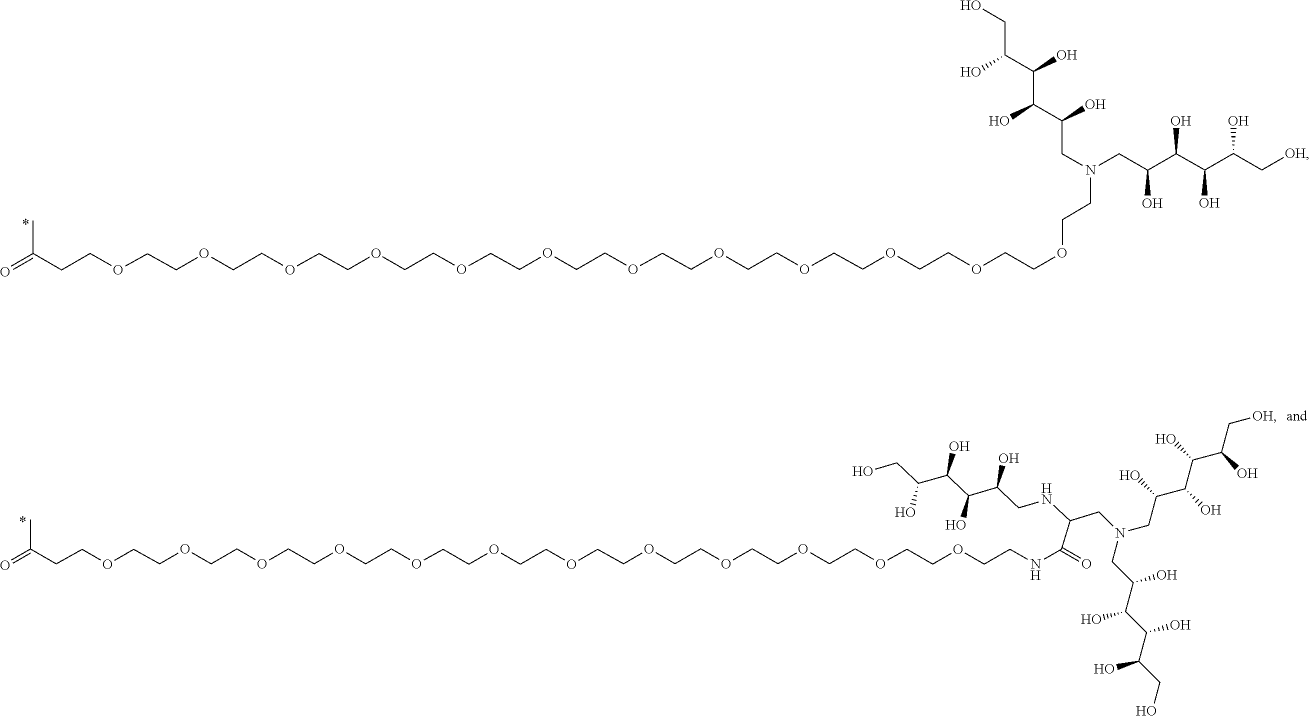 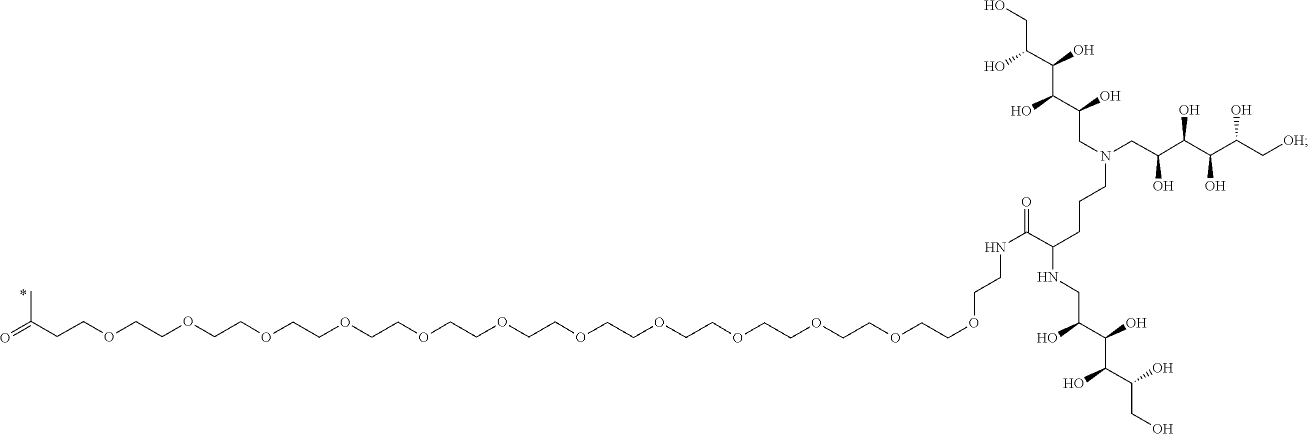
wherein each [symbol] is an attachment site to R^{b}, or to the enzyme-cleavable group.

57. The Linker compound of any one of claims 1 to 6, wherein the Polar group comprises at least one Carboxyl unit having the following formula: 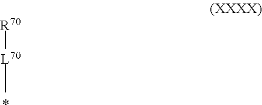
or a stereoisomer or salt thereof, wherein:
(a)
L⁷⁰ is selected from C₁-C₈ alkylene, C₁-C₈ alkylene-C(O)—, —C(O)—C₁-C₈ alkylene-, and —C(O)—C₁-C₈ alkylene-C(O)—, and * is an attachment site to R^{b}, to the enzyme-cleavable group, or to a remainder of the Polar group;
R⁷⁰ is ˜NR⁷¹(R⁷²-R⁷³), wherein R⁷¹ is selected from H, C₁-C₁₂ alkyl, substituted C₁-C₁₂ alkyl, or polyethylene glycol (optionally having 1 to 12 ethylene glycol subunits), R⁷² is a bond or is selected from optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl, and R⁷³ is a carboxyl or polycarboxyl, wherein polycarboxyl comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide; or
(b)
L⁷⁰ is selected from C₁-C₈ alkylene, C₁-C₈ alkylene-C(O)—, —C(O)—C₁-C₈ alkylene-, and —C(O)—C₁-C₈ alkylene-C(O)—, and * is an attachment site to R^{b}, to the enzyme-cleavable group, or to a remainder of the Polar group;
R⁷⁰ is ˜NR⁷¹(R⁷⁵—(R⁷³)₂), wherein R⁷¹ is selected from H, C₁-C₁₂ alkyl, substituted C₁-C₁₂ alkyl, or polyethylene glycol (optionally having 1 to 12 ethylene glycol subunits), R⁷⁵ is a branched optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl and each R⁷³ is independently carboxyl or polycarboxyl, wherein polycarboxyl comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide; or
(c)
L⁷⁰ is selected from C₁-C₈ alkylene, C₁—C alkylene-C(O)—, —C(O)—C₁—C alkylene-, and —C(O)—C₁-C₈ alkylene-C(O)—, and * is an attachment site to R^{b}, to the enzyme-cleavable group, or to a remainder of the Polar group;
R⁷⁰ is —N(R⁷⁴-R⁷³)(R⁷²-R⁷³), wherein R⁷² and R⁷⁴ are each independently selected from optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl, and each R⁷³ is independently carboxyl or polycarboxyl, wherein the polycarboxyl comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide.

58. The Linker compound of any one of claims 1 to 57, comprising a Polar group including the Polymer unit and a Sugar unit.

59. The Linker compound of any o one f claims 1 to 57, comprising a Polar group including at least two Polymer units.

60. The Linker compound of any one of claims 1 to 57, comprising a Polar group including the Polymer unit and a Carboxyl unit.

61. The Linker compound of any one of claims 1 to 57, comprising at least two Polar groups.

62. The Linker compound of any one of claims 1 to 57, comprising a Polar group including the Polymer unit, the Sugar unit and the Carboxyl unit.

63. The Linker compound of any one of claims 1 to 57, comprising a Polar group including at least two Polymer units, at least one Sugar unit and at least one Carboxyl unit.

64. The Linker compound of any one of claims 1 to 57, wherein the enzyme-cleavable group comprises at least two amino acid units.

65. The Linker compound of any one of claims 1 to 64, comprising at least one of the Polar group attached to the enzyme-cleavable group.

66. The Linker compound of any one of claims 1-64, having one of the following structures: 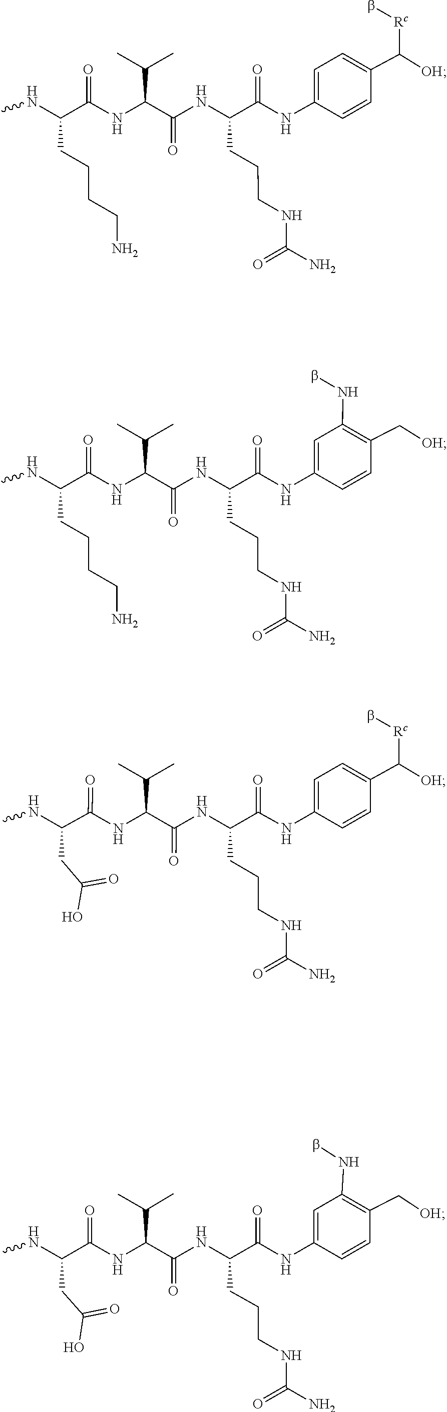 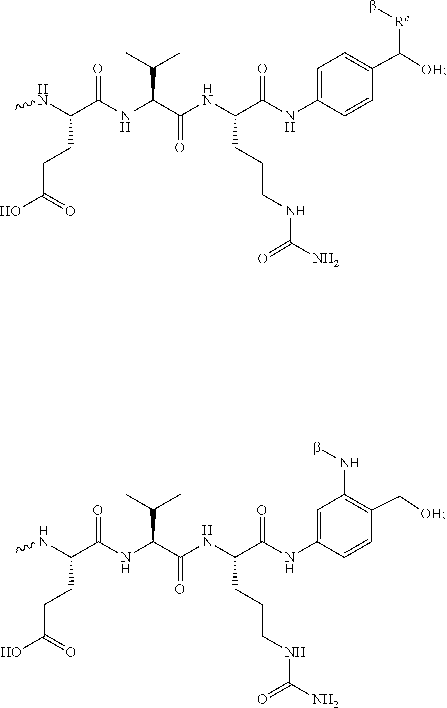 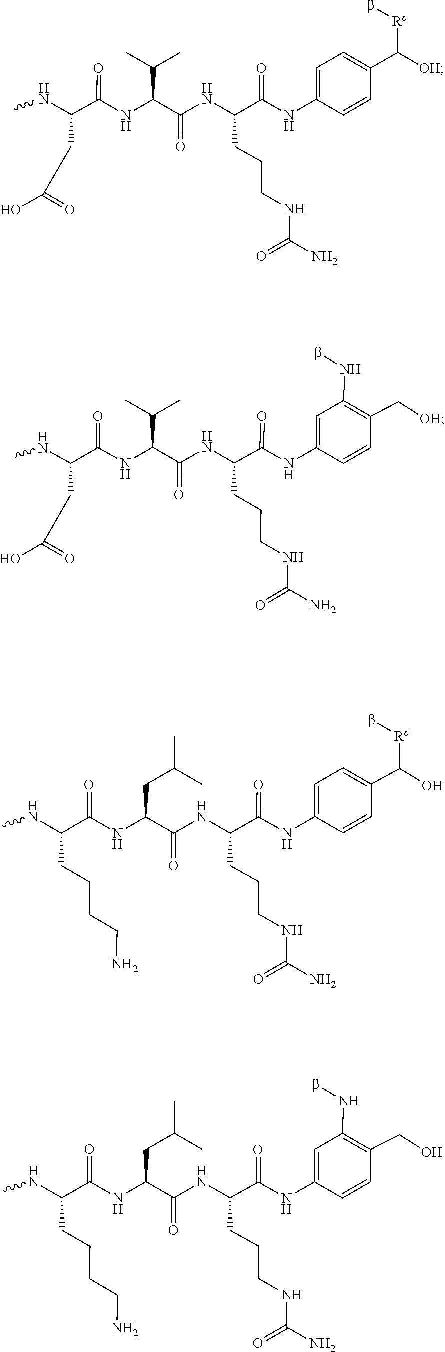 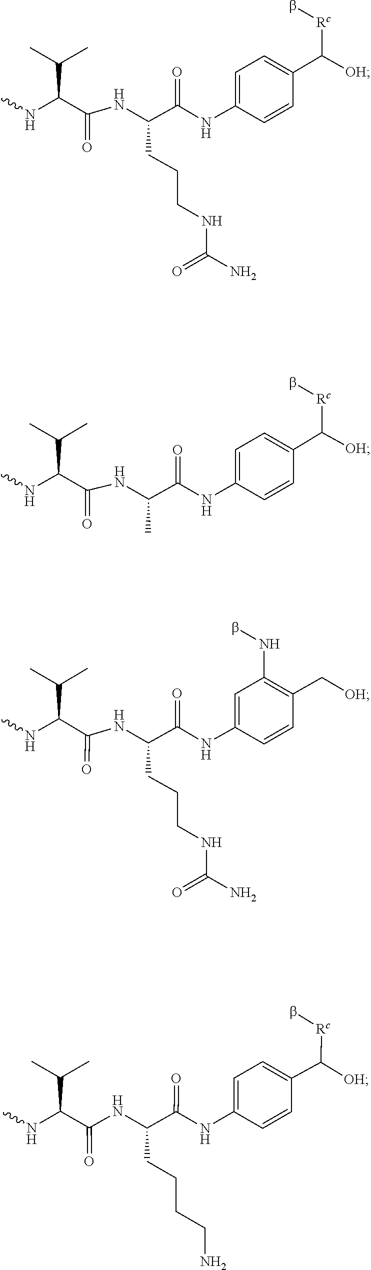 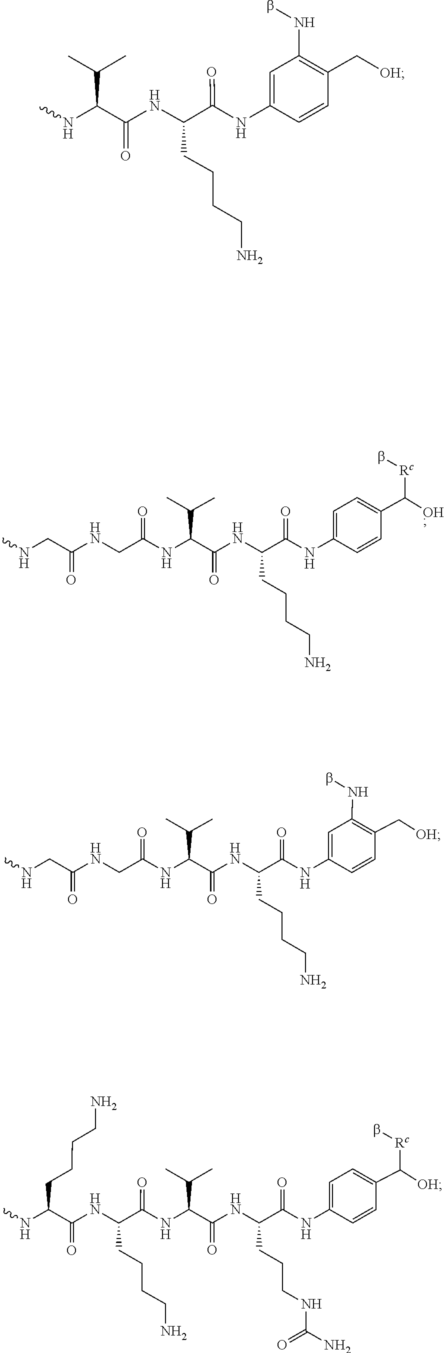 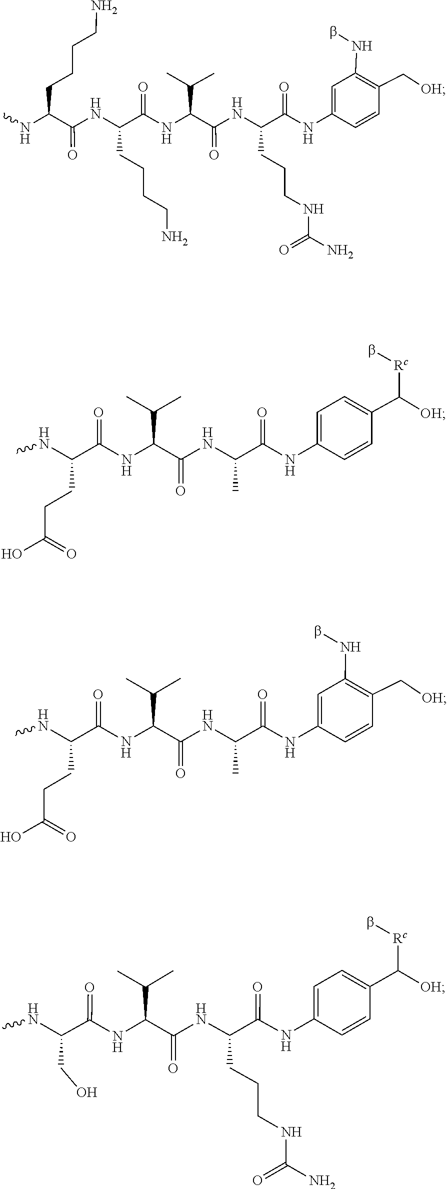 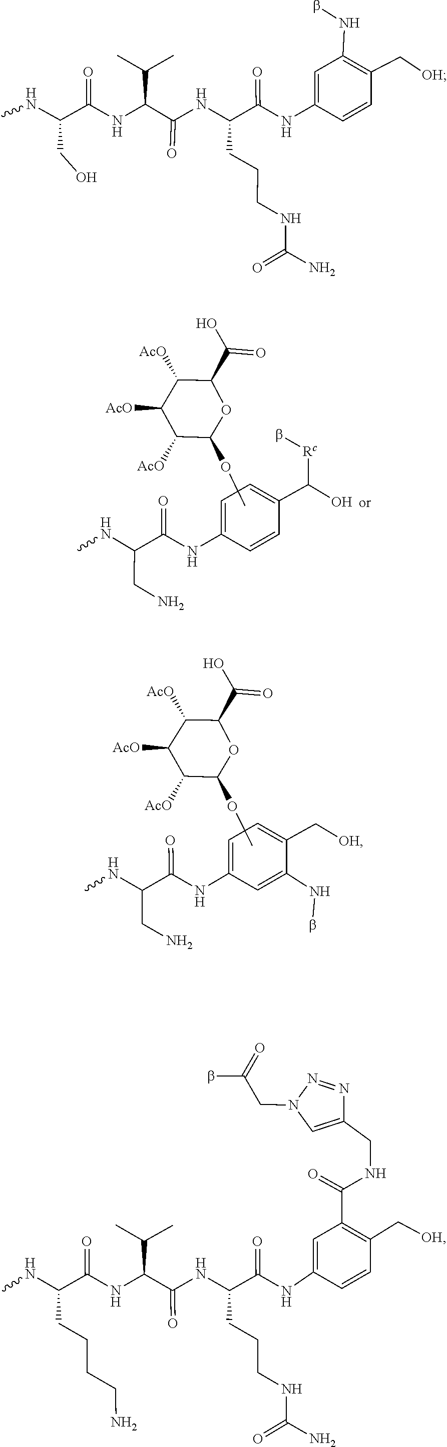 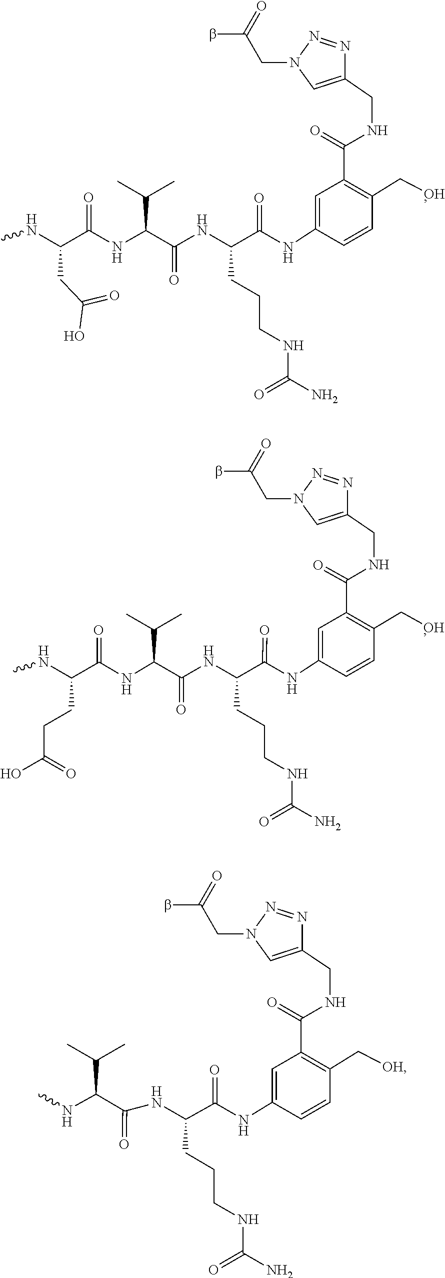 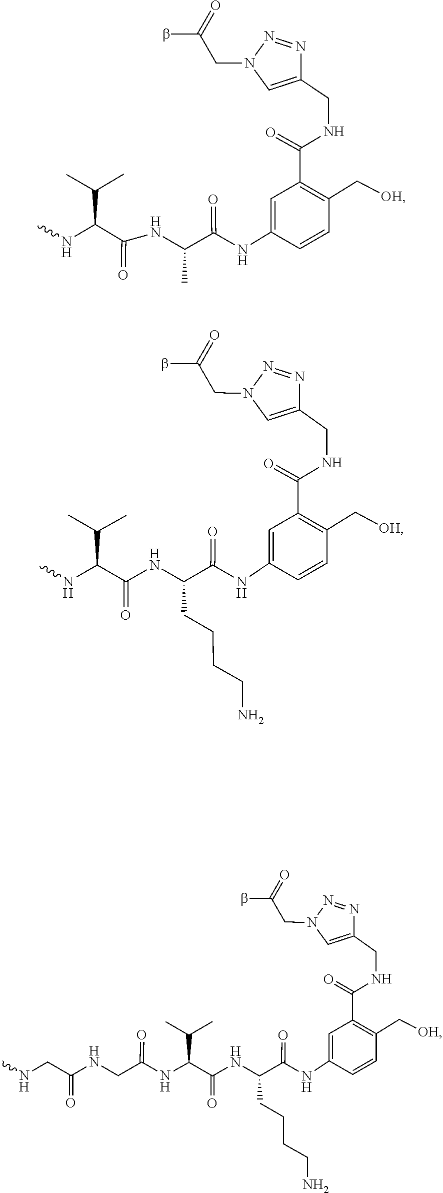 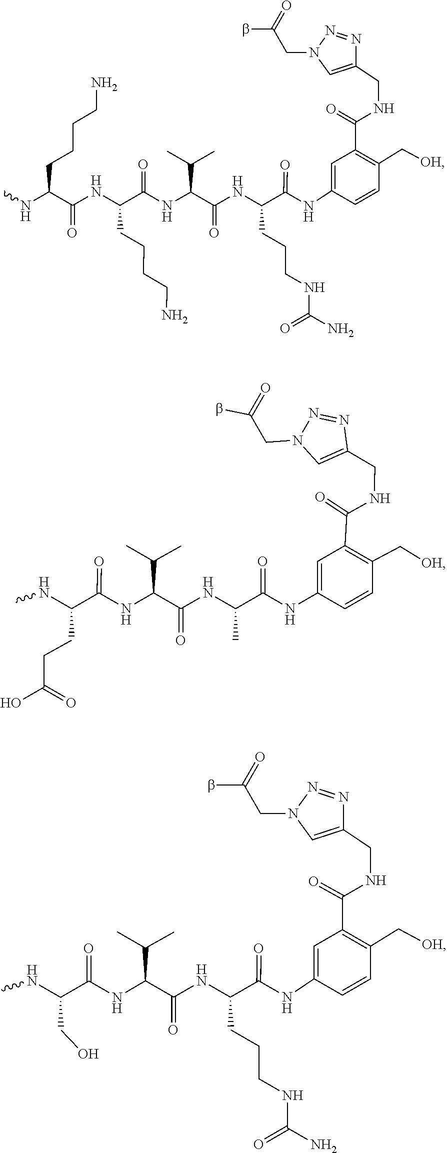 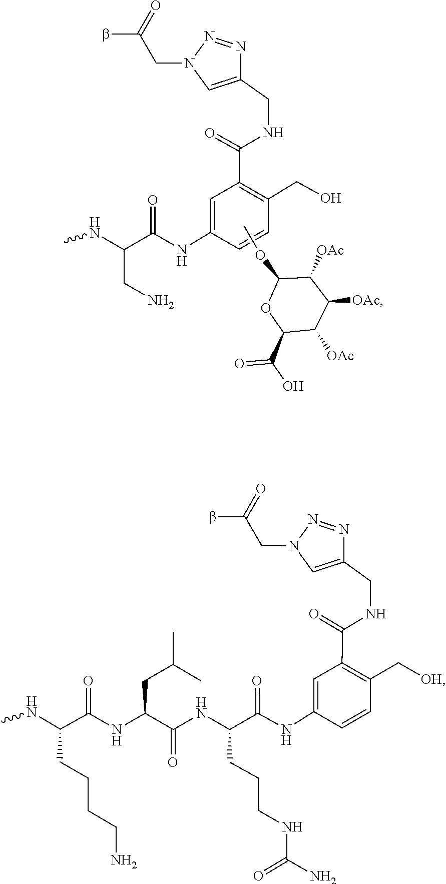
wherein
R^{c} is a bond or C₁₋₆ alkylene;
the wavy line on the amino group indicates an attachment site for a Stretcher group or, prior to attachment to the Stretcher group, indicates H;
β—is the attachment site to the at least one Polar group; and
the benzylic H on the benzylic OH is optionally replaced with a bond to at least one of the Drug units or to the linking group attached to at least one of the Drug units.

67. The Linker compound of any one of claims 1-66, wherein the enzyme-cleavable group comprises a peptide that is cleavable by an intracellular protease.

68. The Linker compound of claim 67, wherein the intracellular protease is Cathepsin B.

69. The Linker compound of claim 68, wherein the enzyme-cleavable group comprises a cleavable peptide including a valine-citrulline peptide, a valine-alanine peptide, a valine-lysine peptide, a phenylalanine-lysine peptide, or a glycine-glycine-phenylalanine-glycine peptide.

70. The Linker compound of any one of claims 67-69, comprising one of the following structures: [chemical structure] 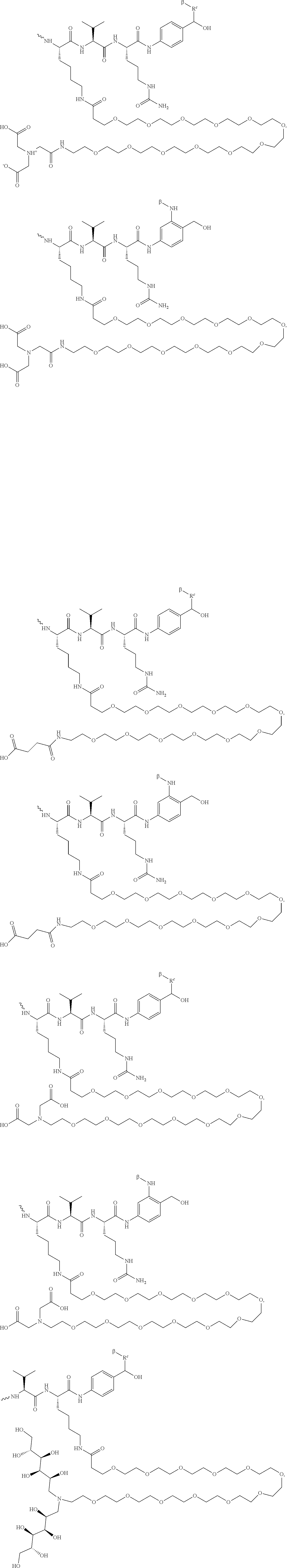 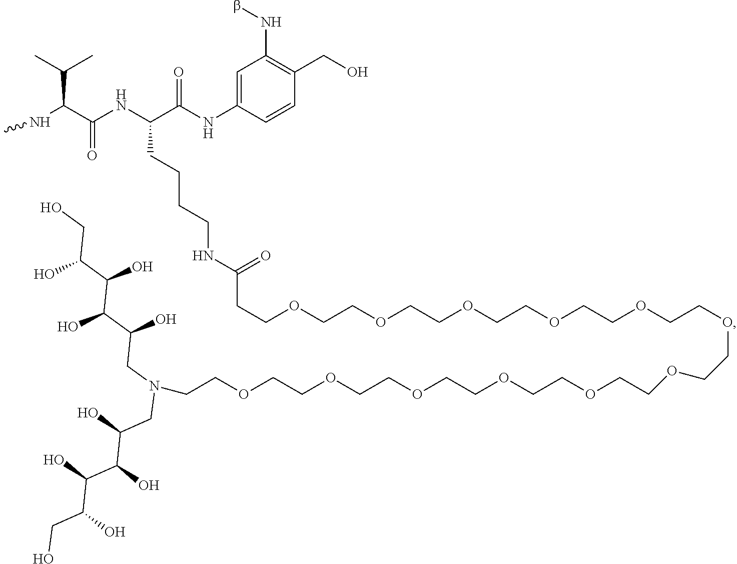 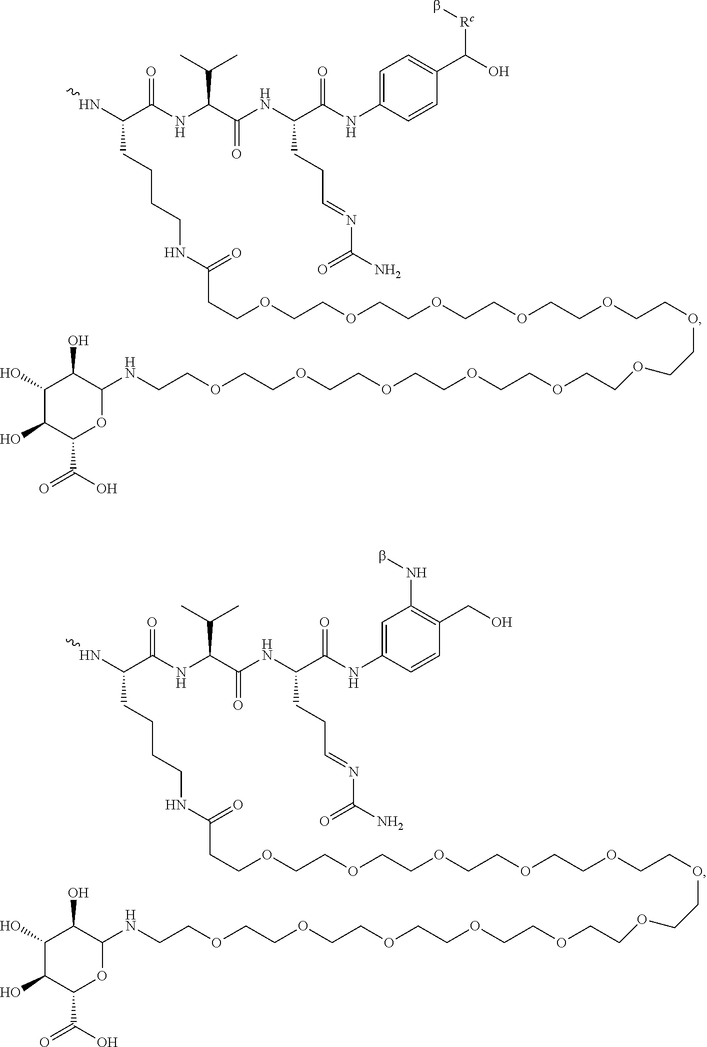 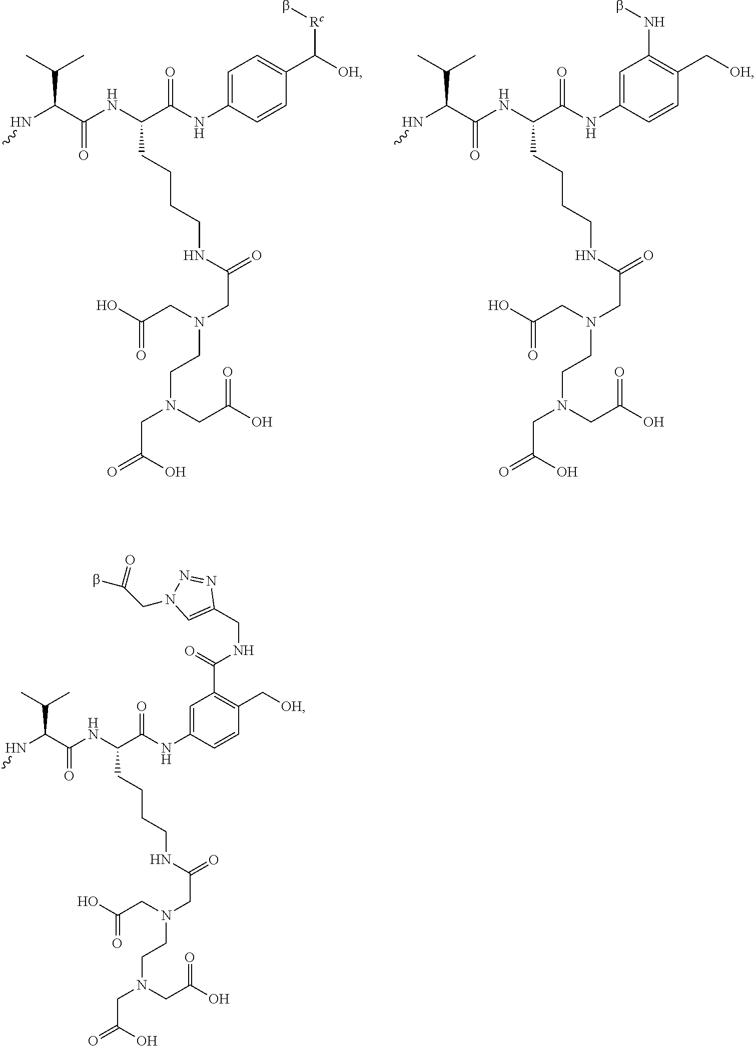 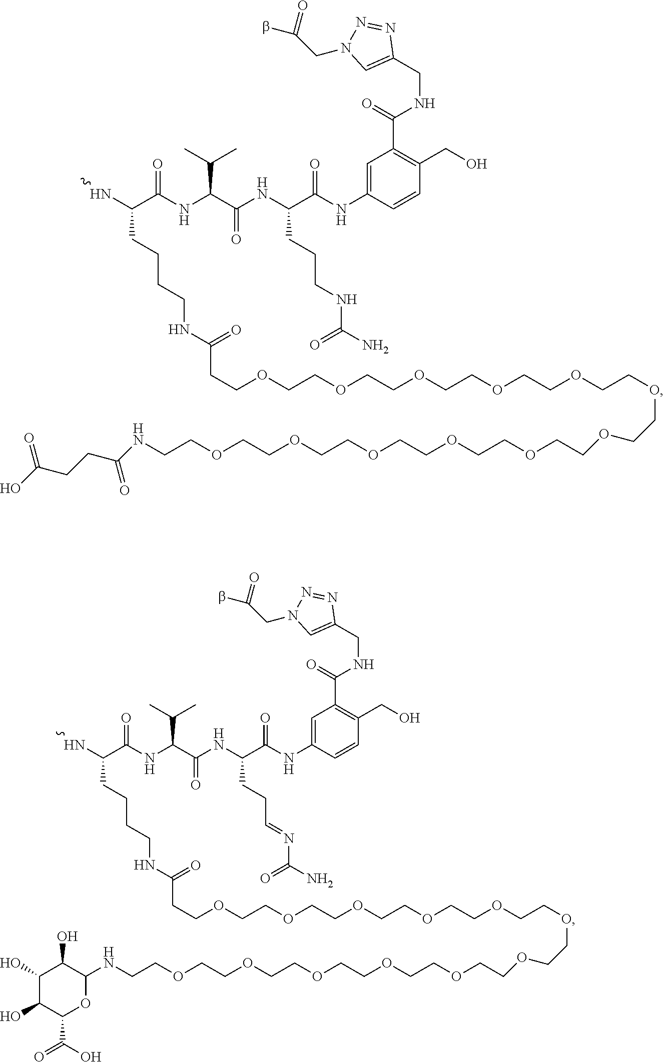 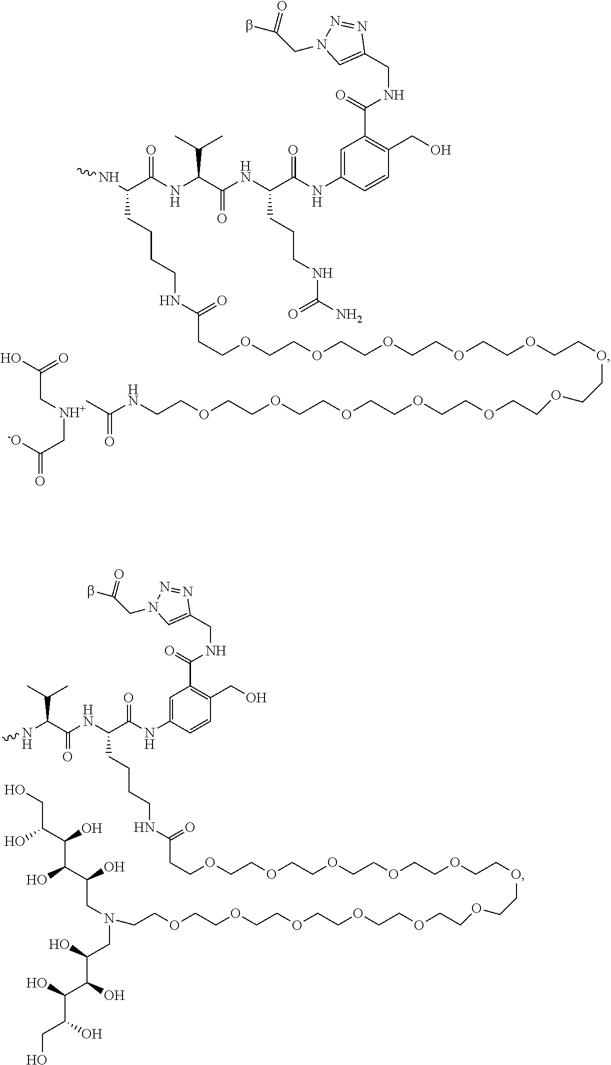 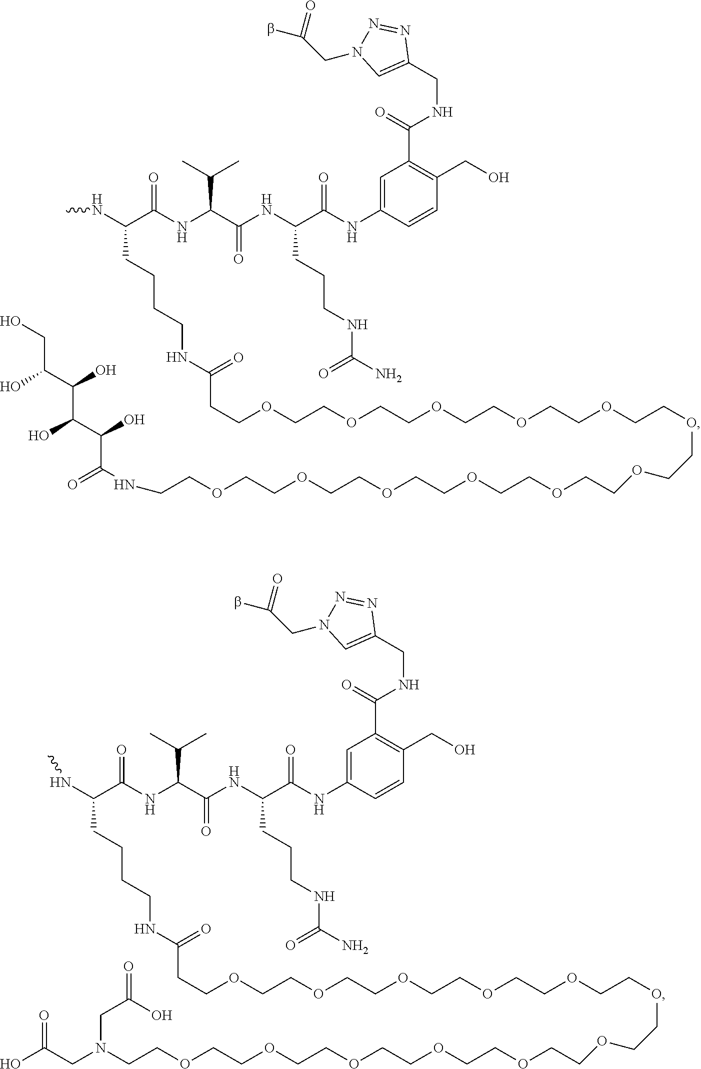 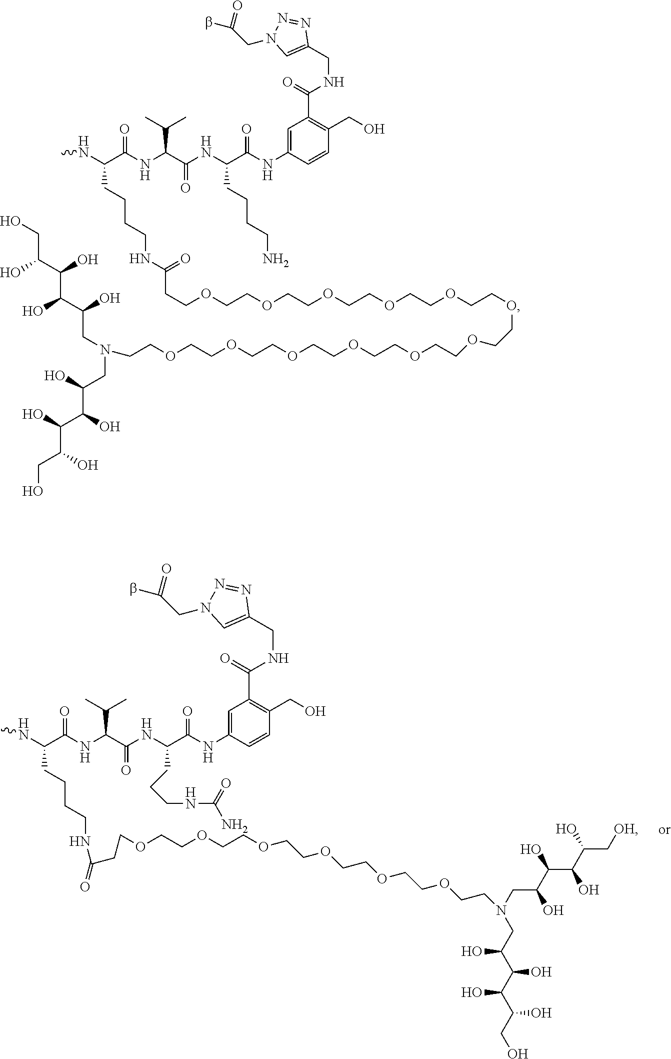 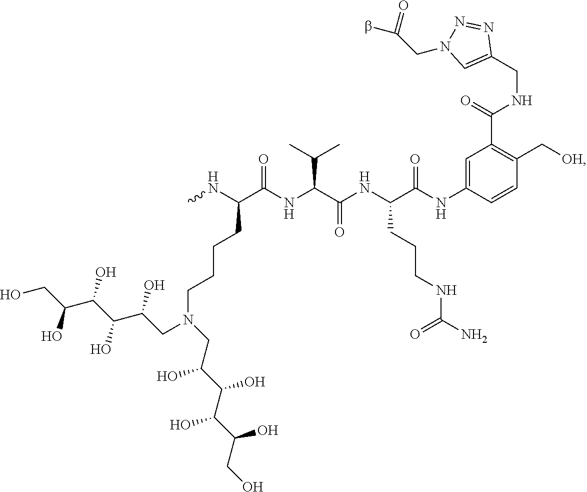
wherein
R^{c} is a bond or C₁₋₆ alkylene;
the wavy line on the amino group indicates an attachment site for the Stretcher group; or, prior to attachment to the Stretcher group, indicates H;
β—is the attachment site to the at least one Polar group; and
the H on the benzylic OH is optionally replaced with a bond to at least one of the Drug units or to the attachment site to at least one of the Drug units.

71. The Linker compound of any one of claims 1-5, having one of the following structures: 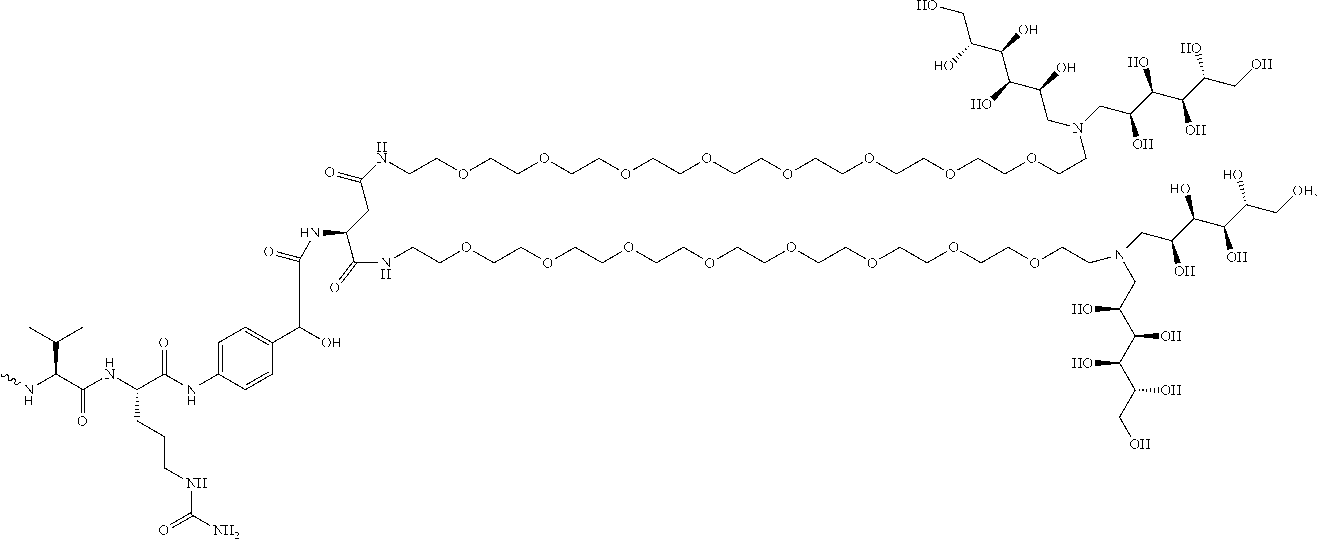 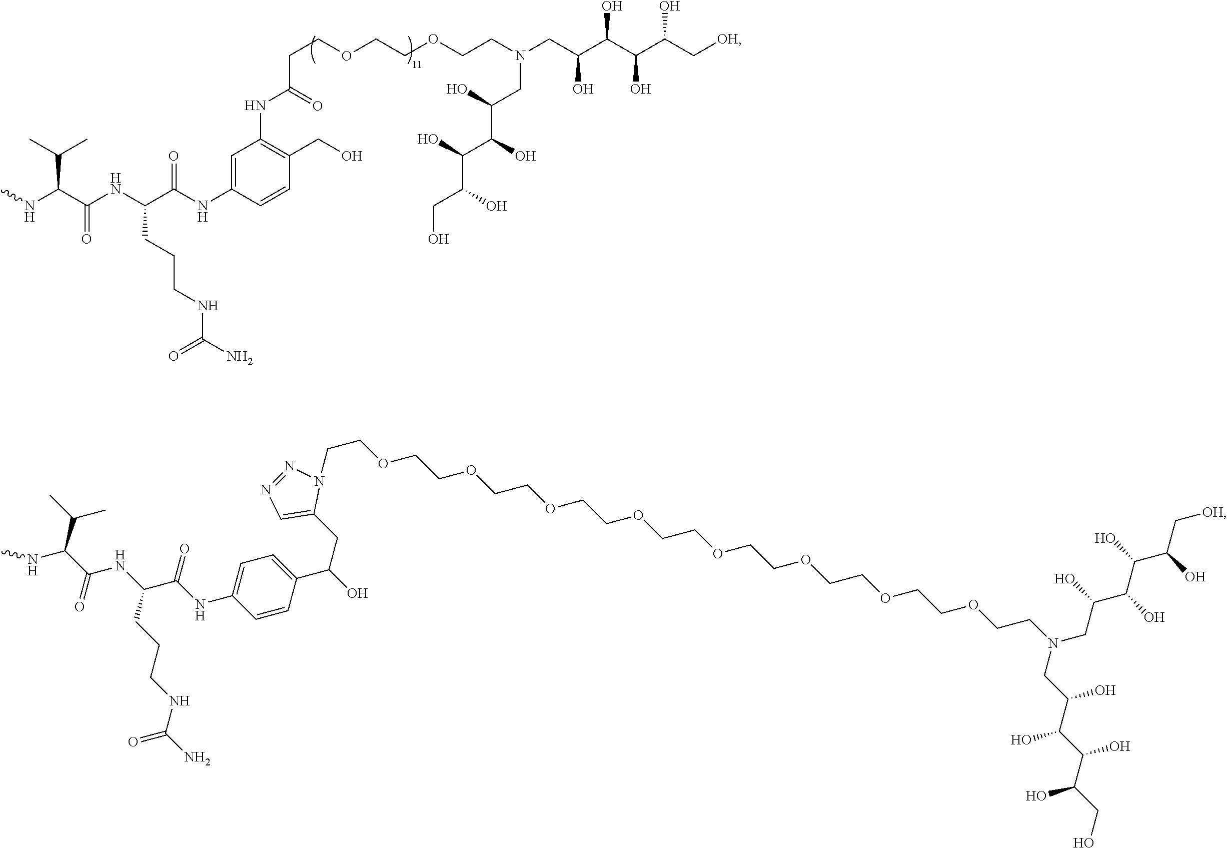 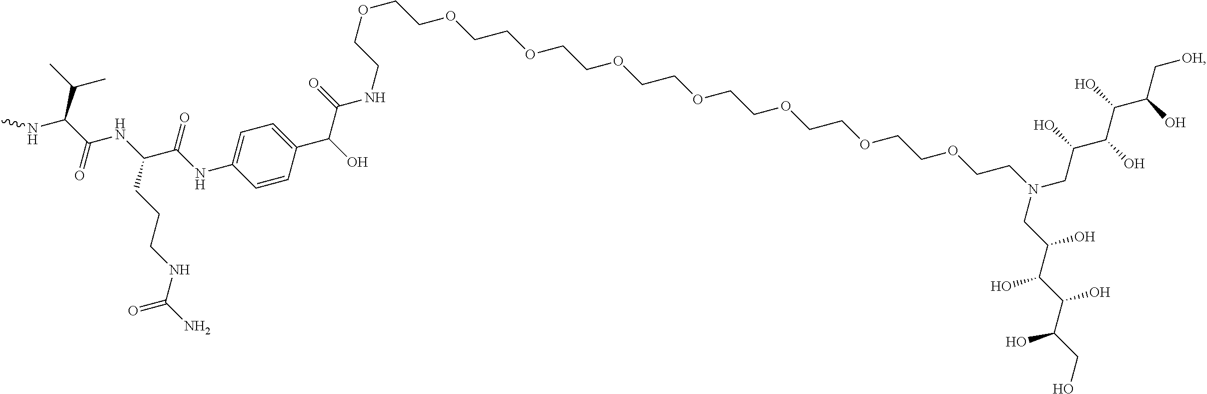 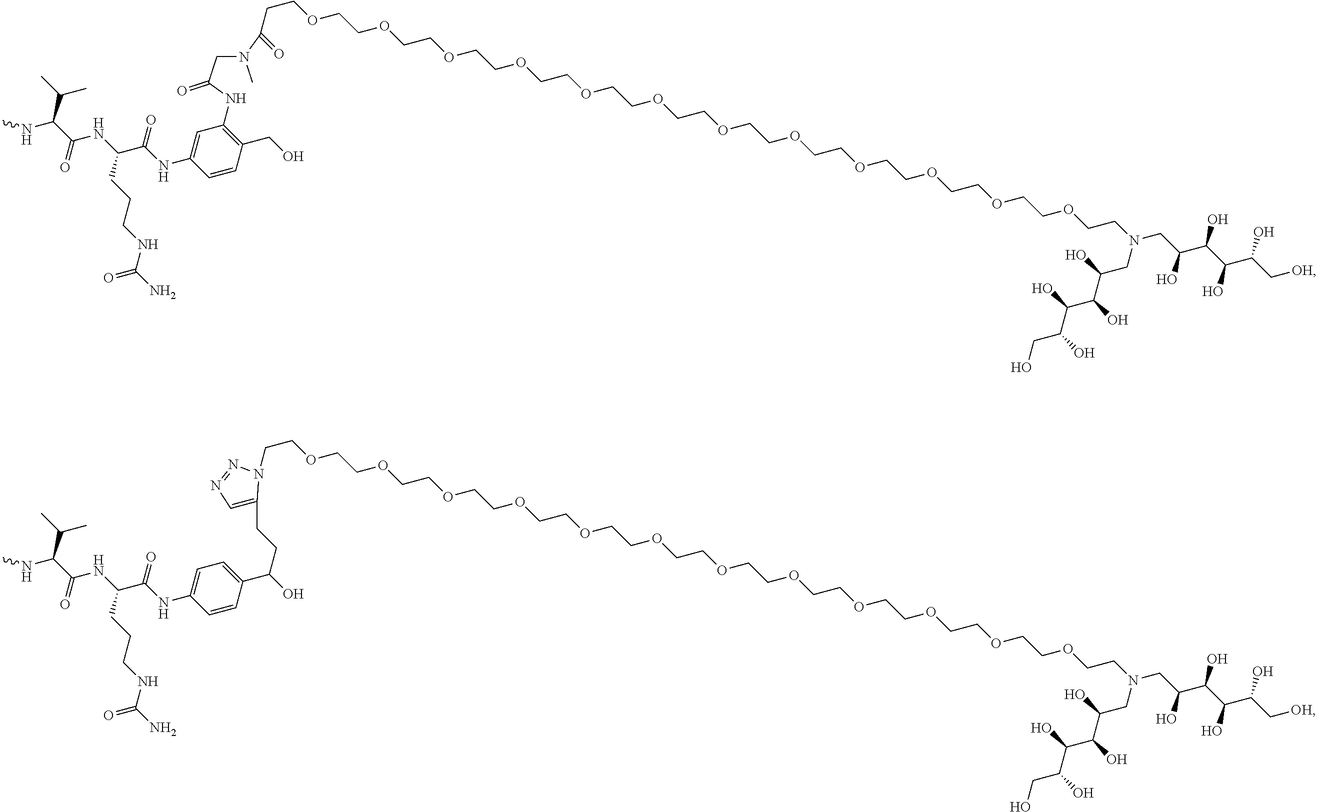 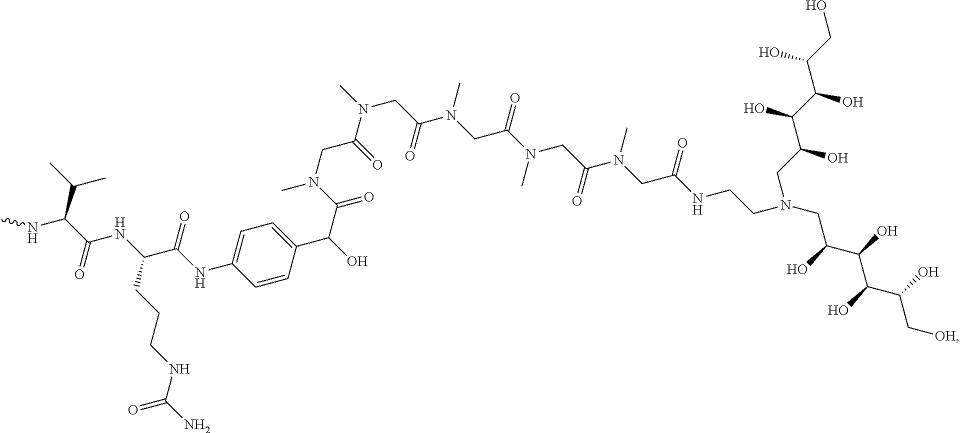 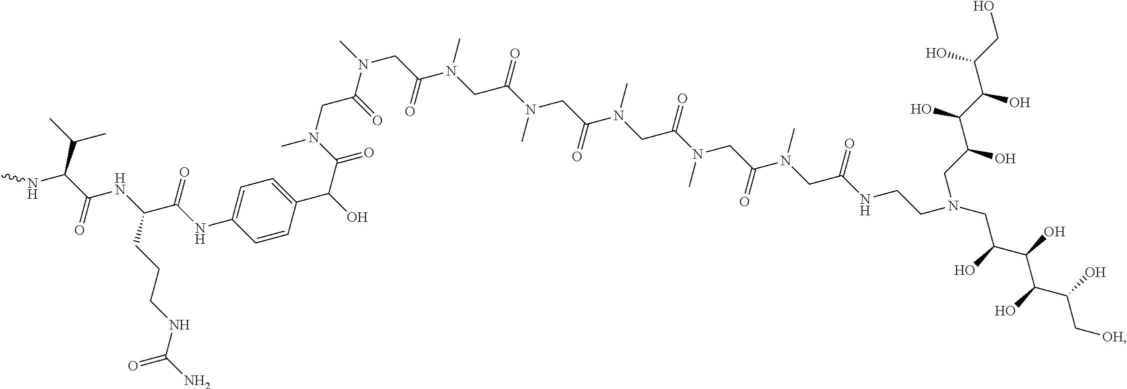 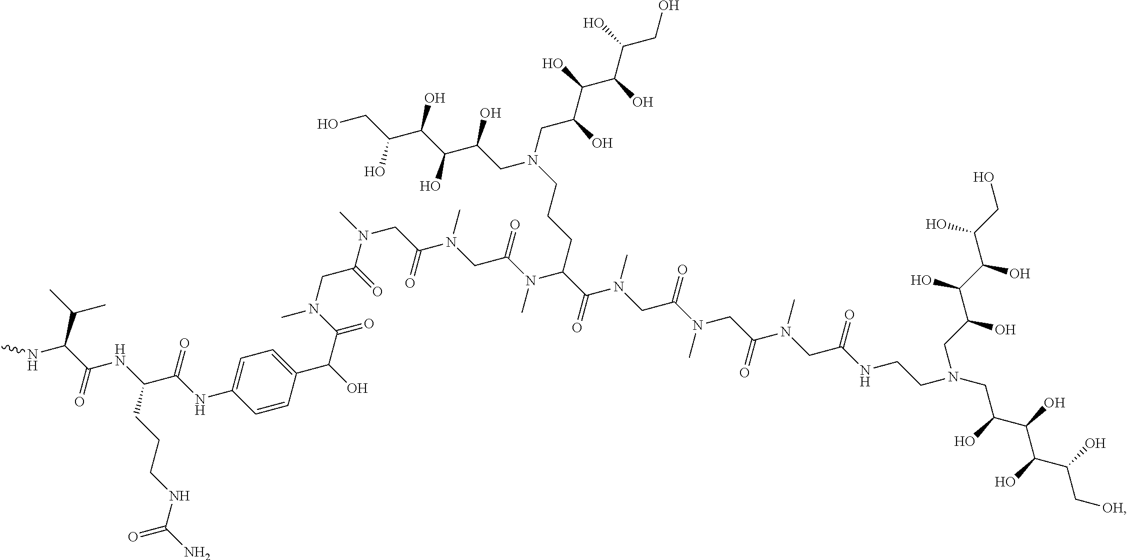 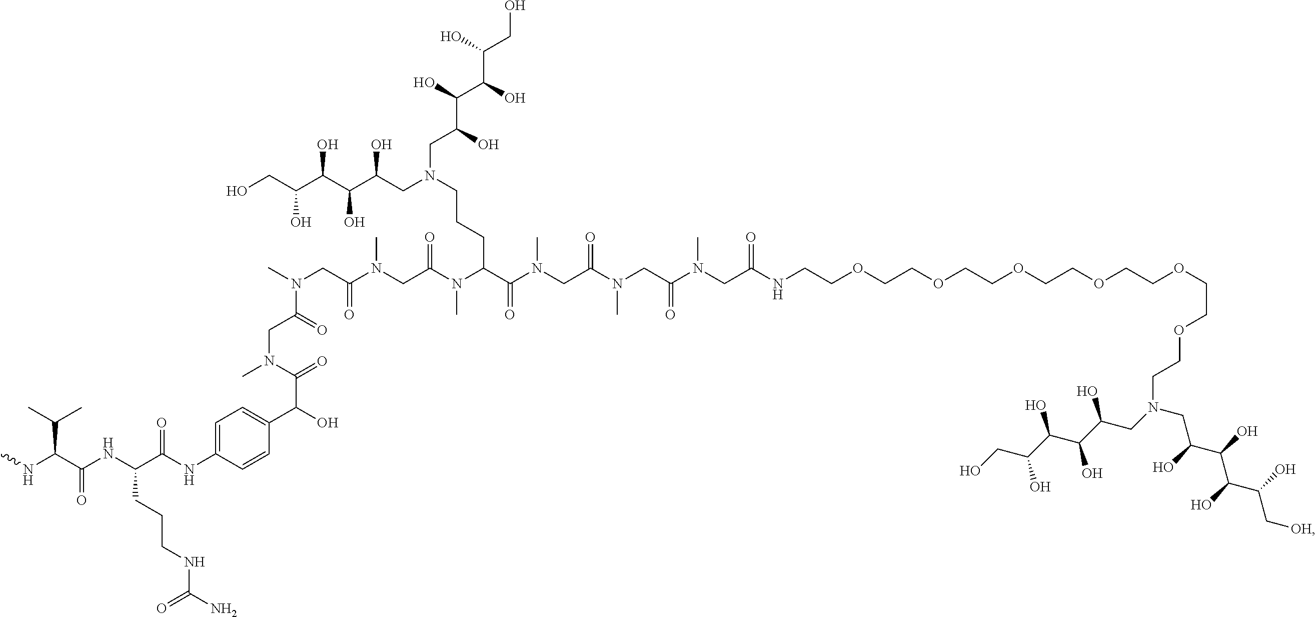 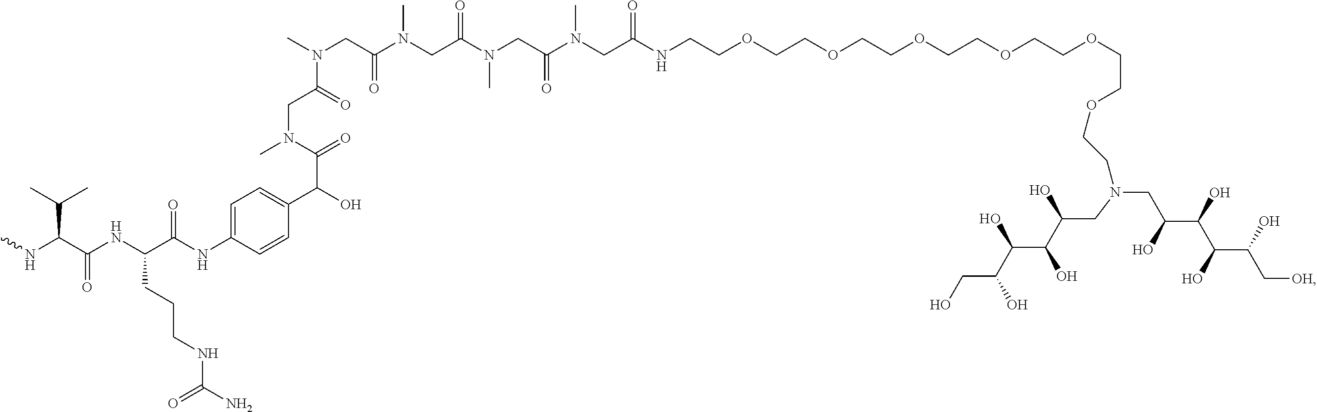 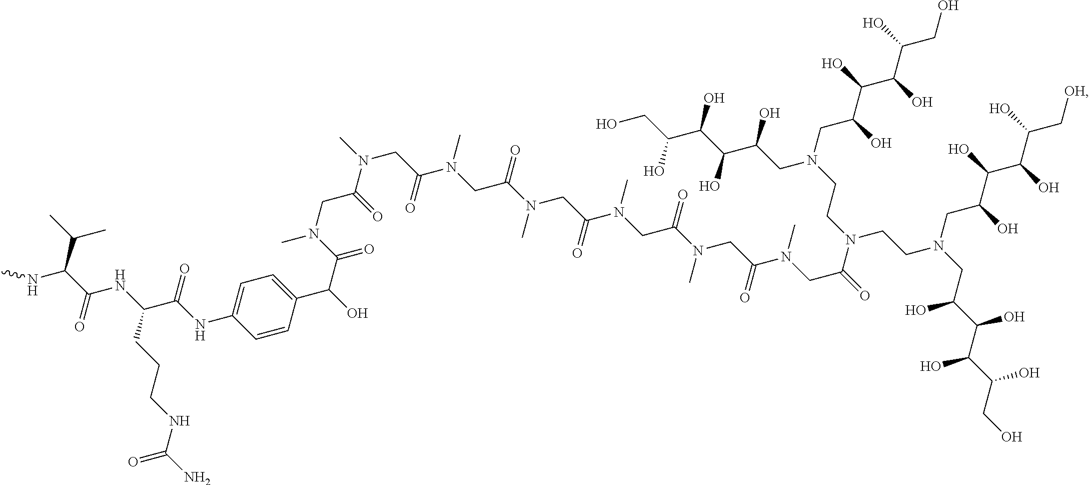 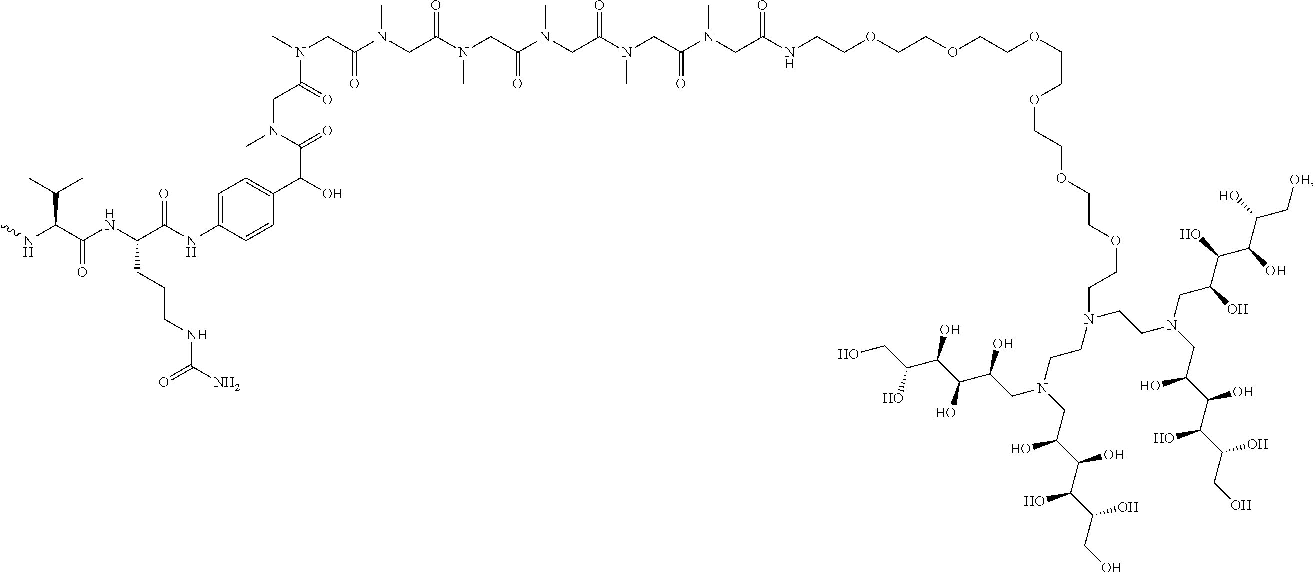 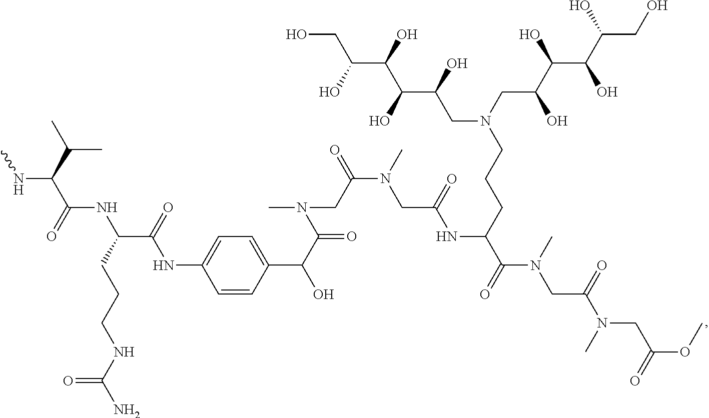 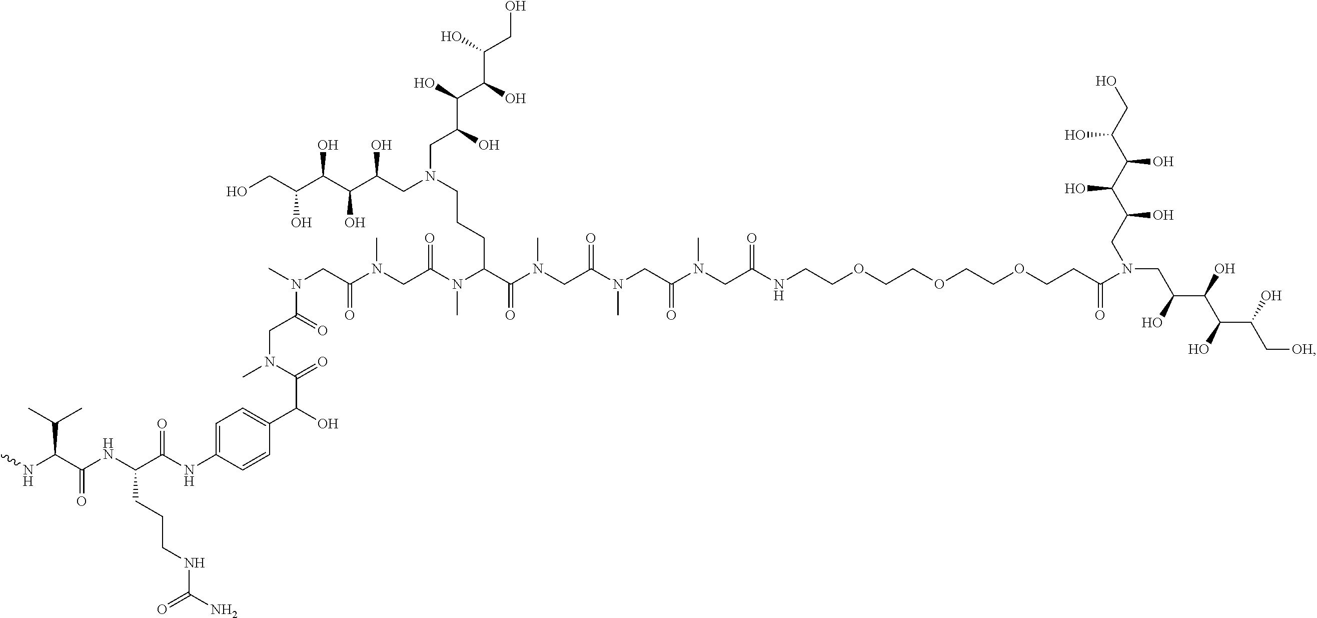 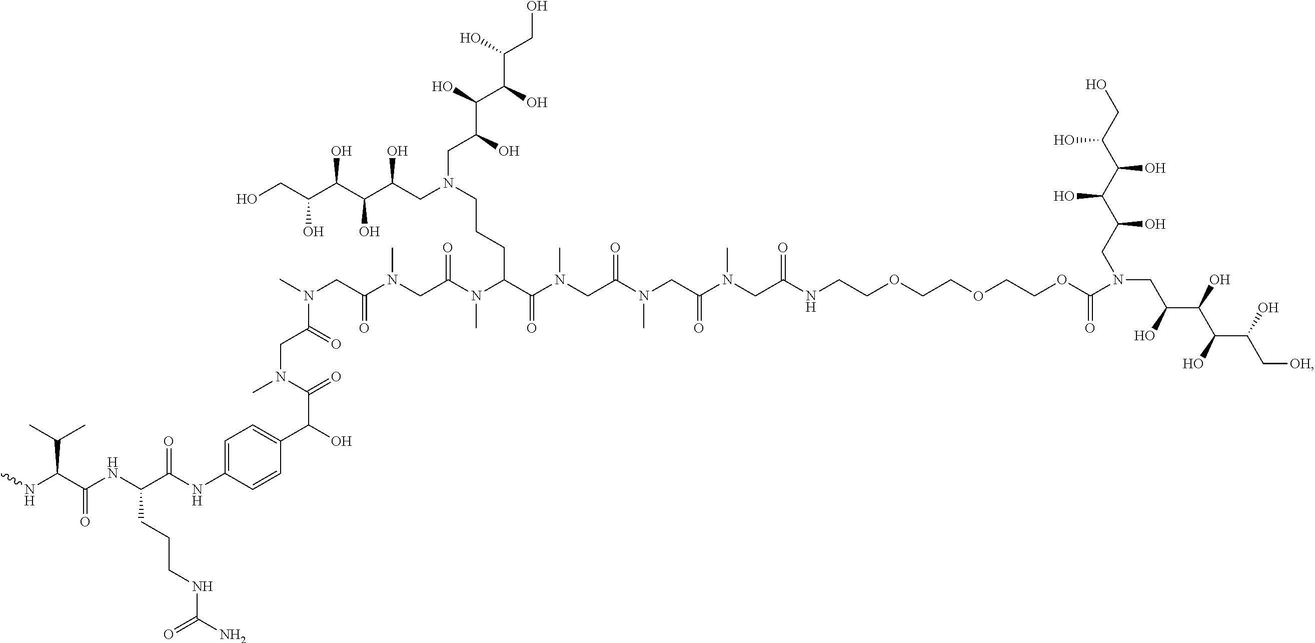 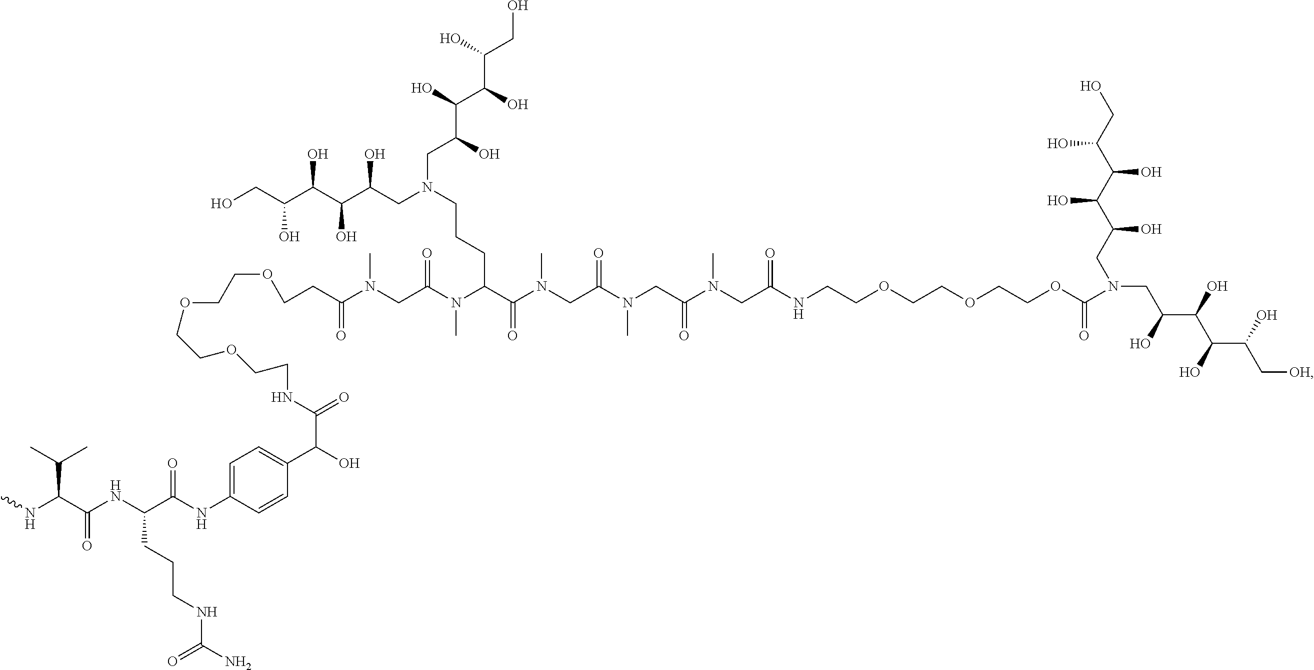 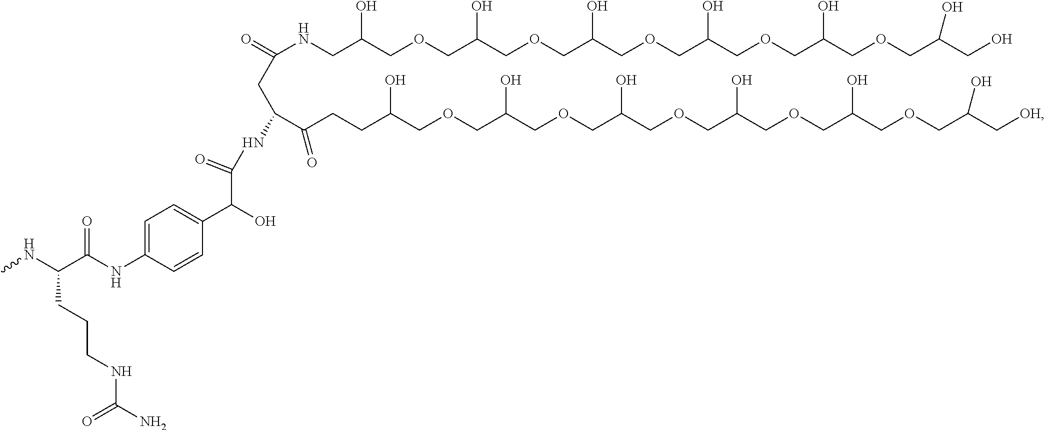 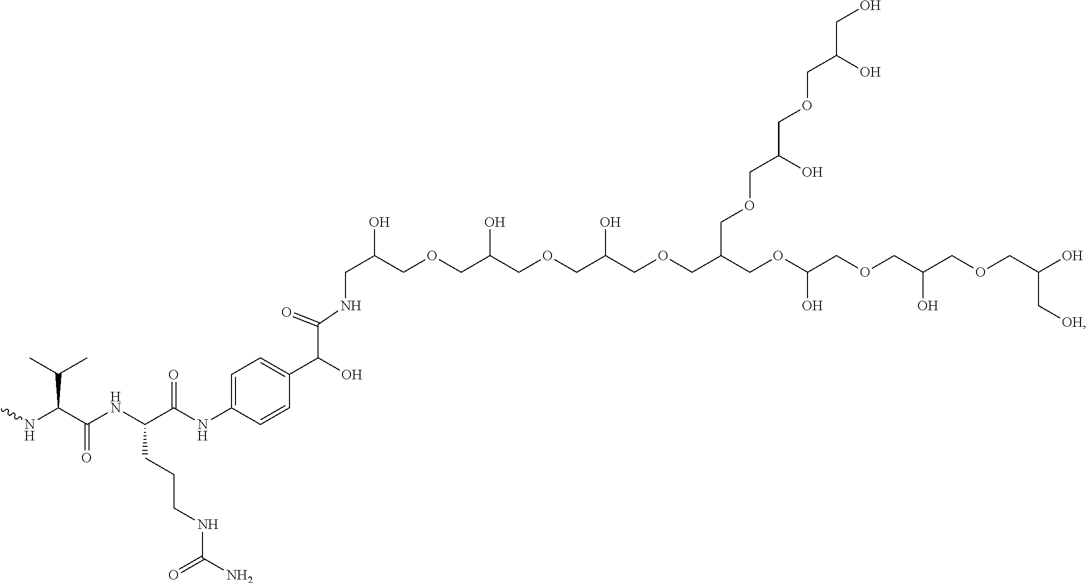 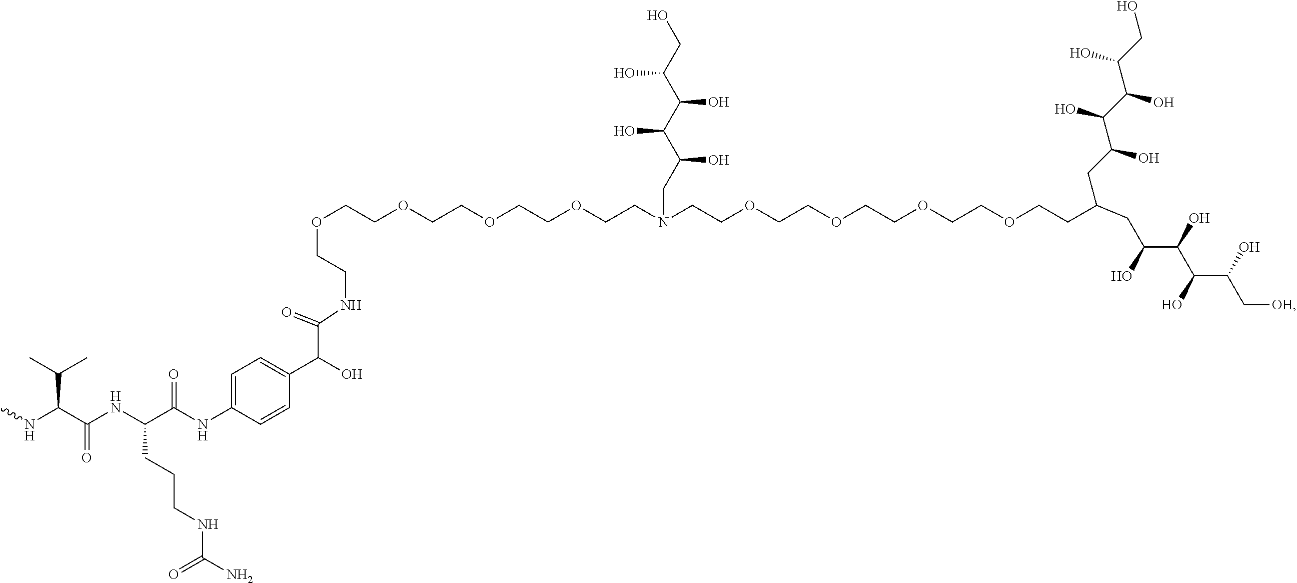 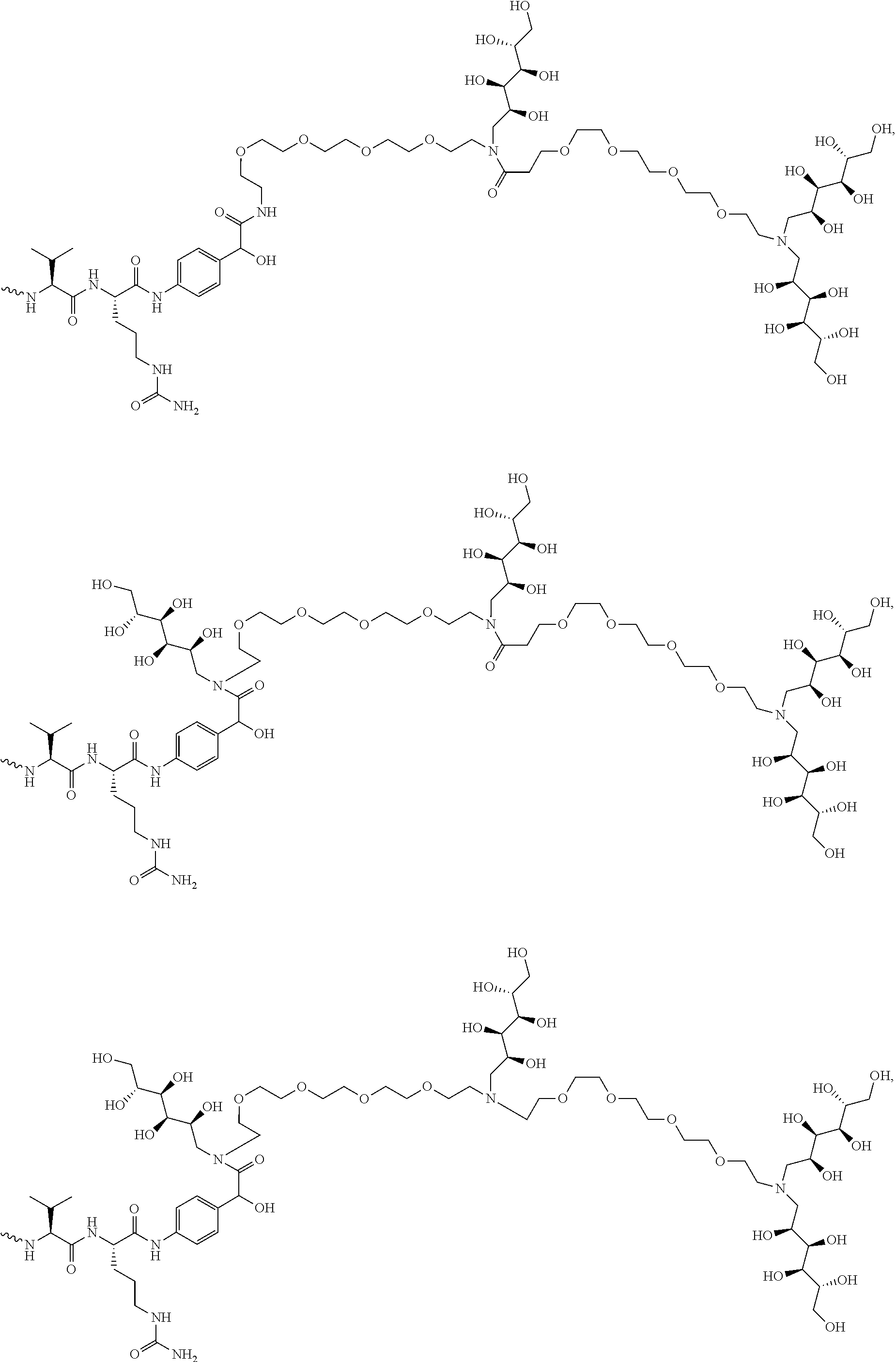 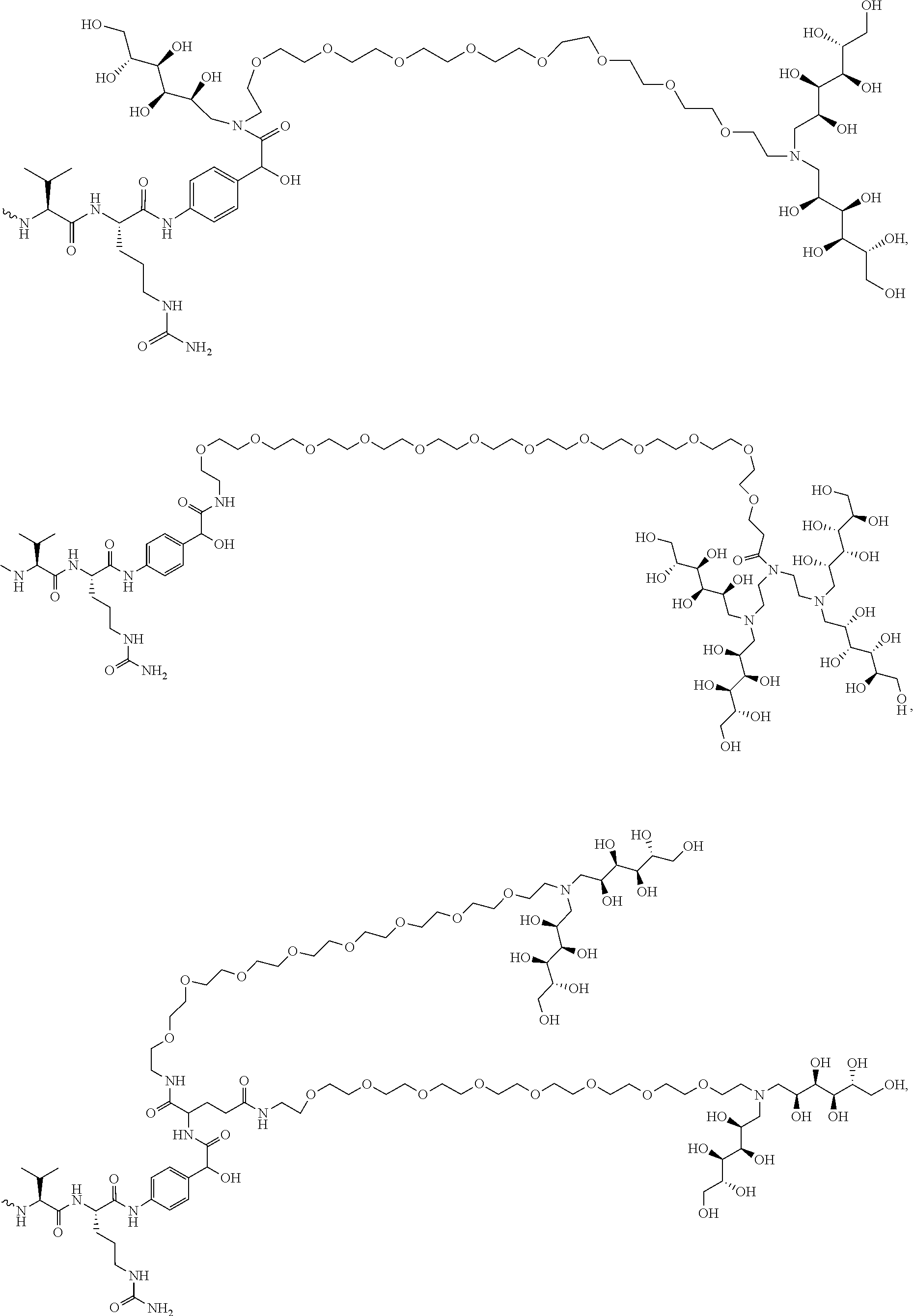 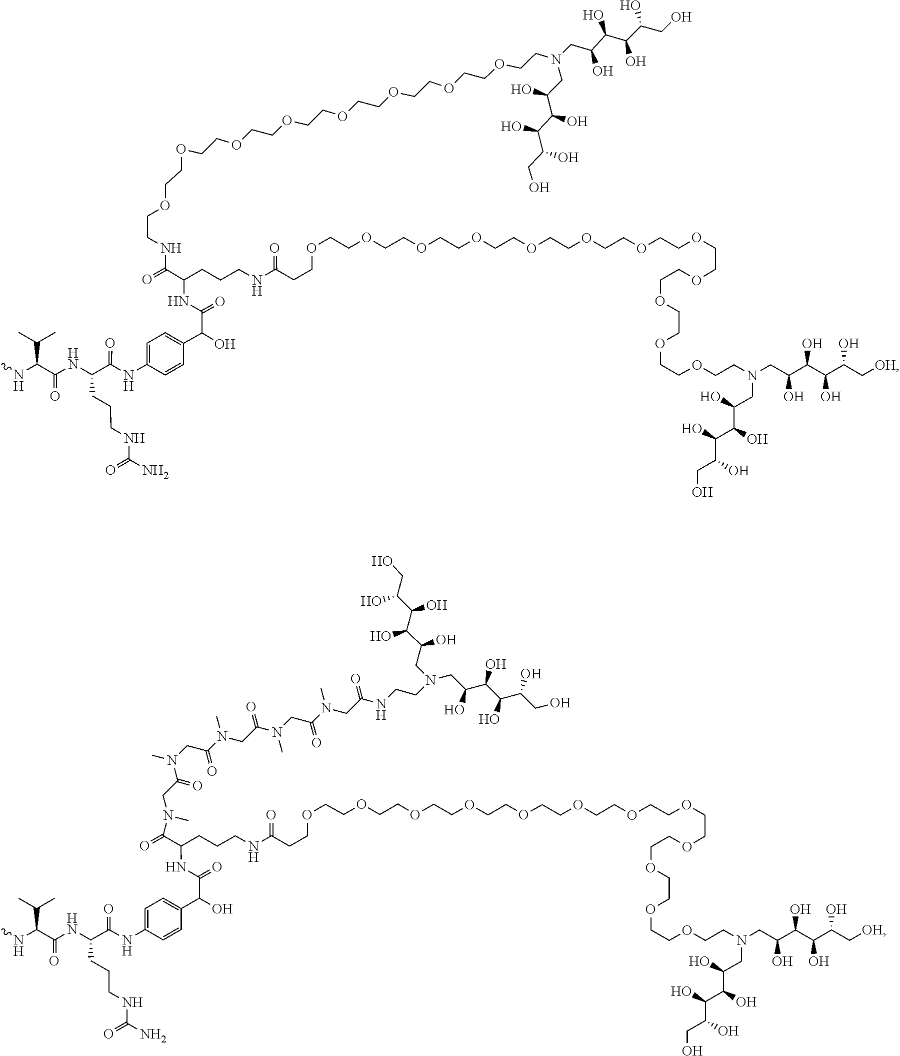 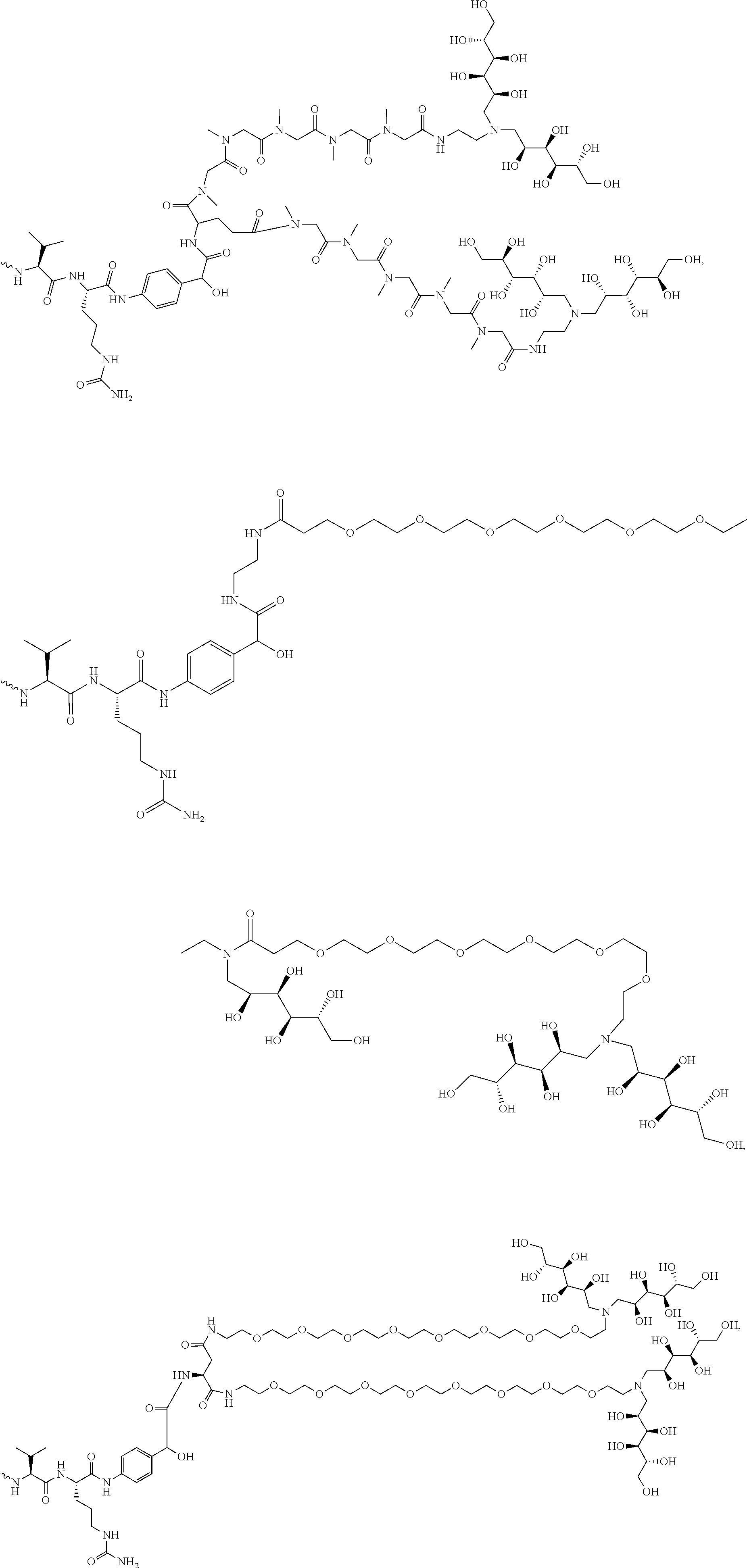 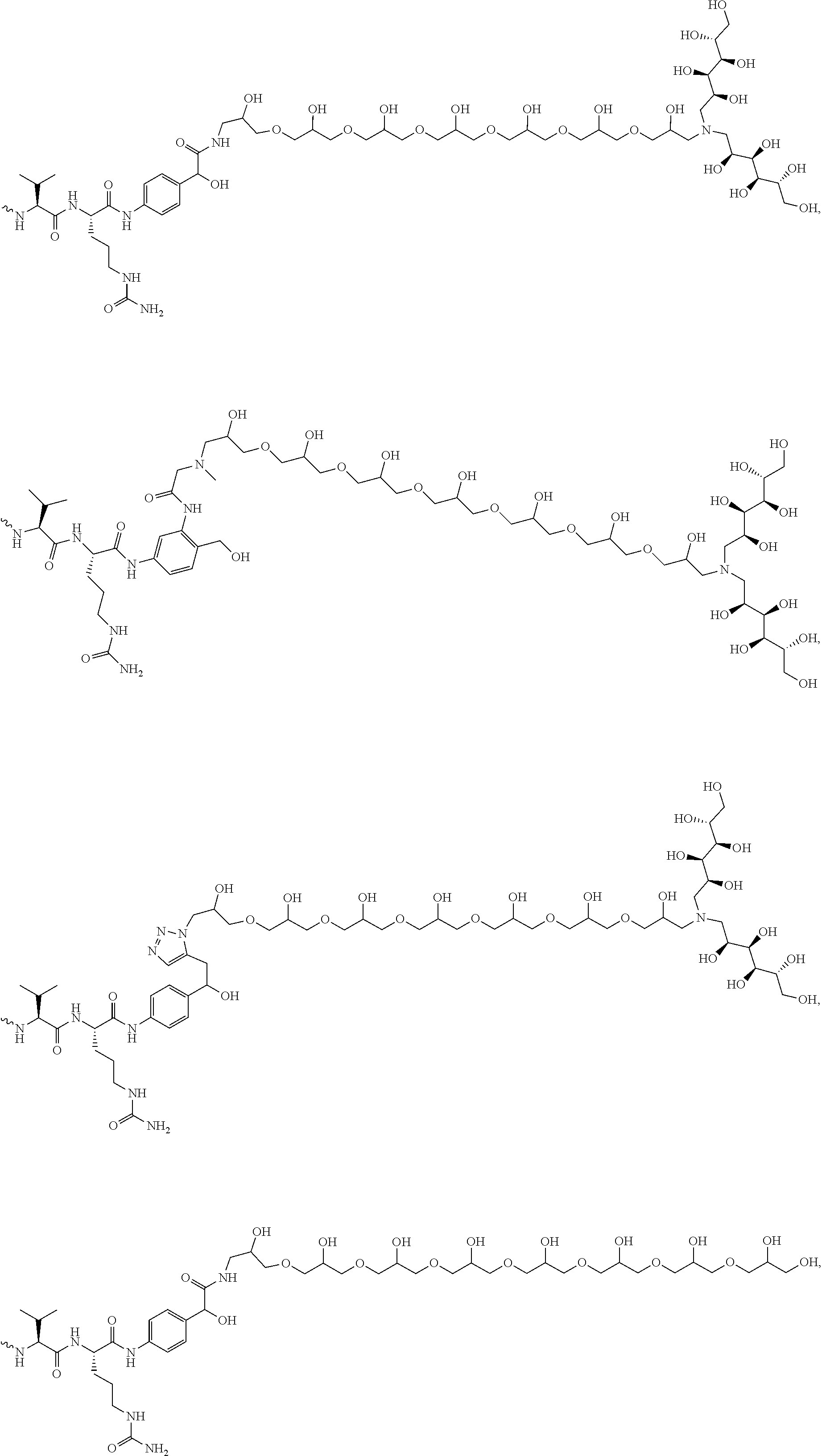 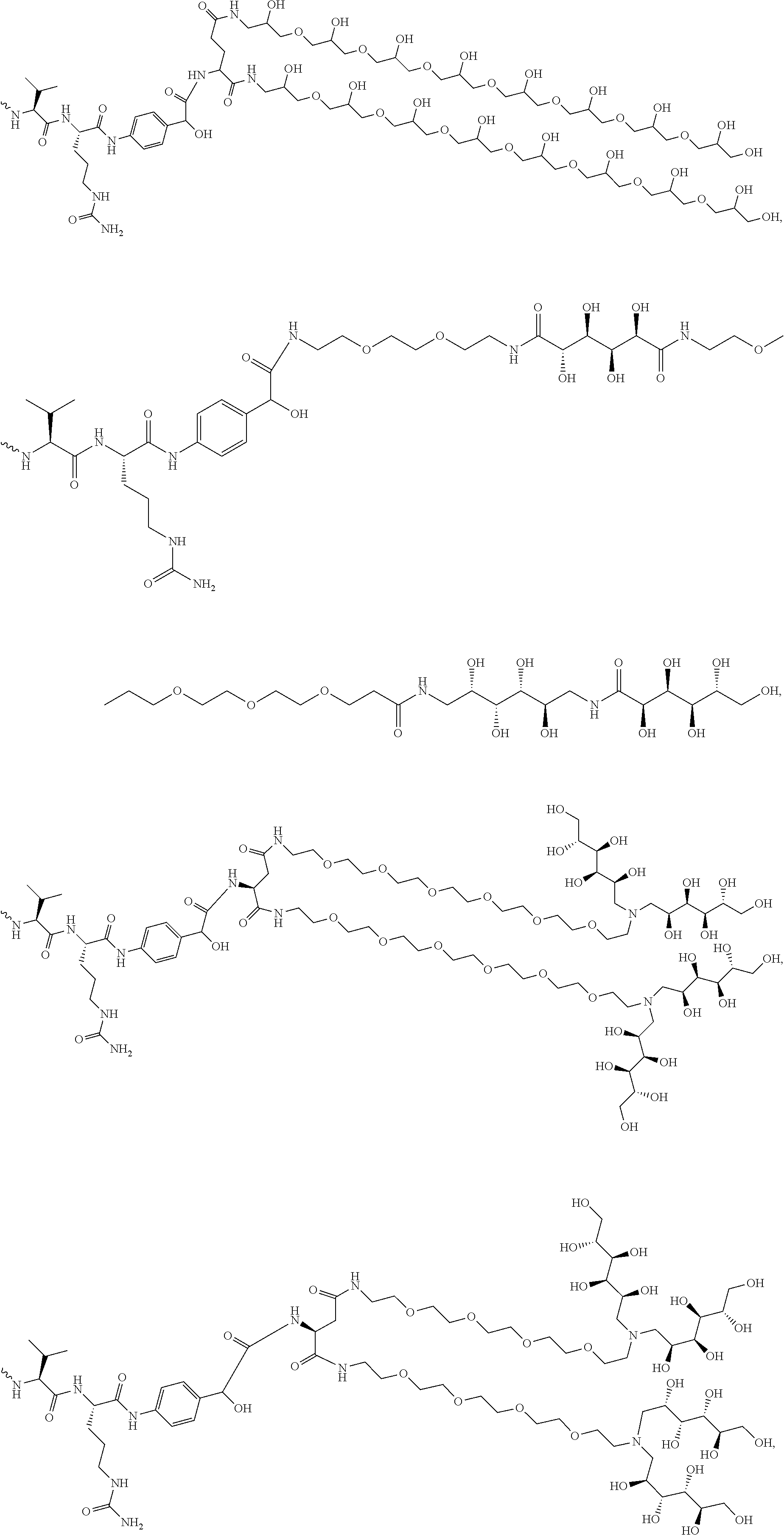 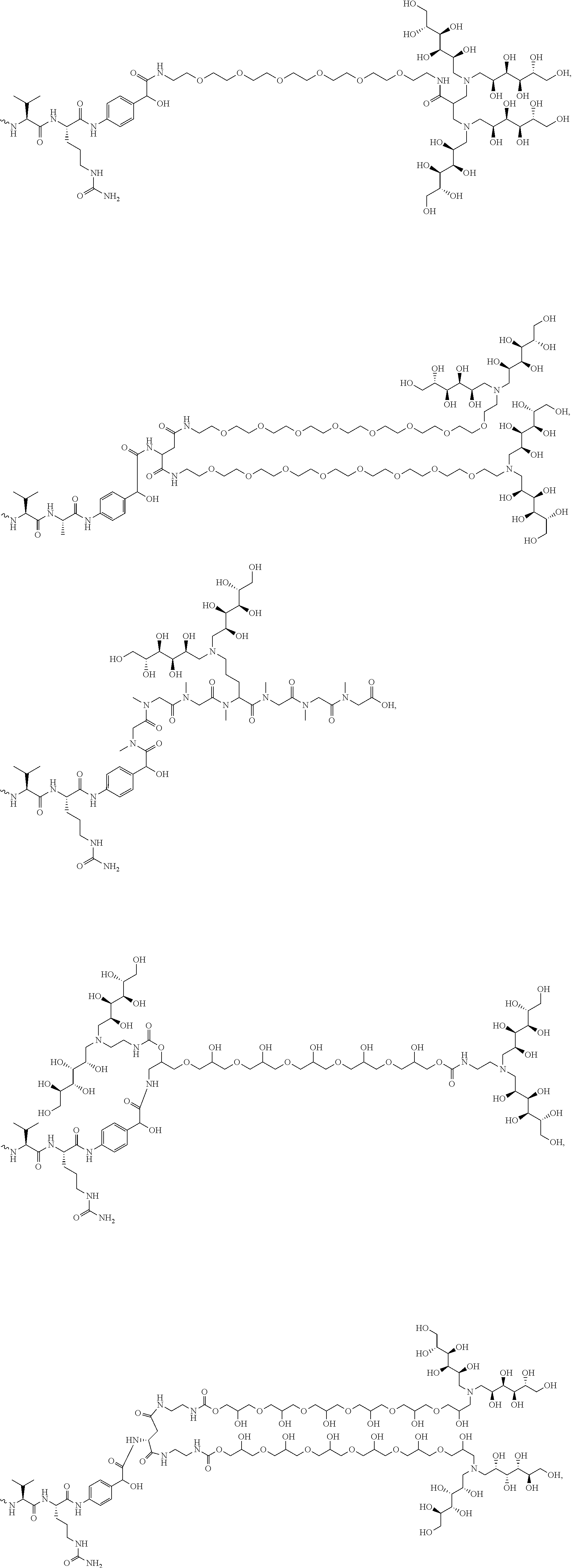 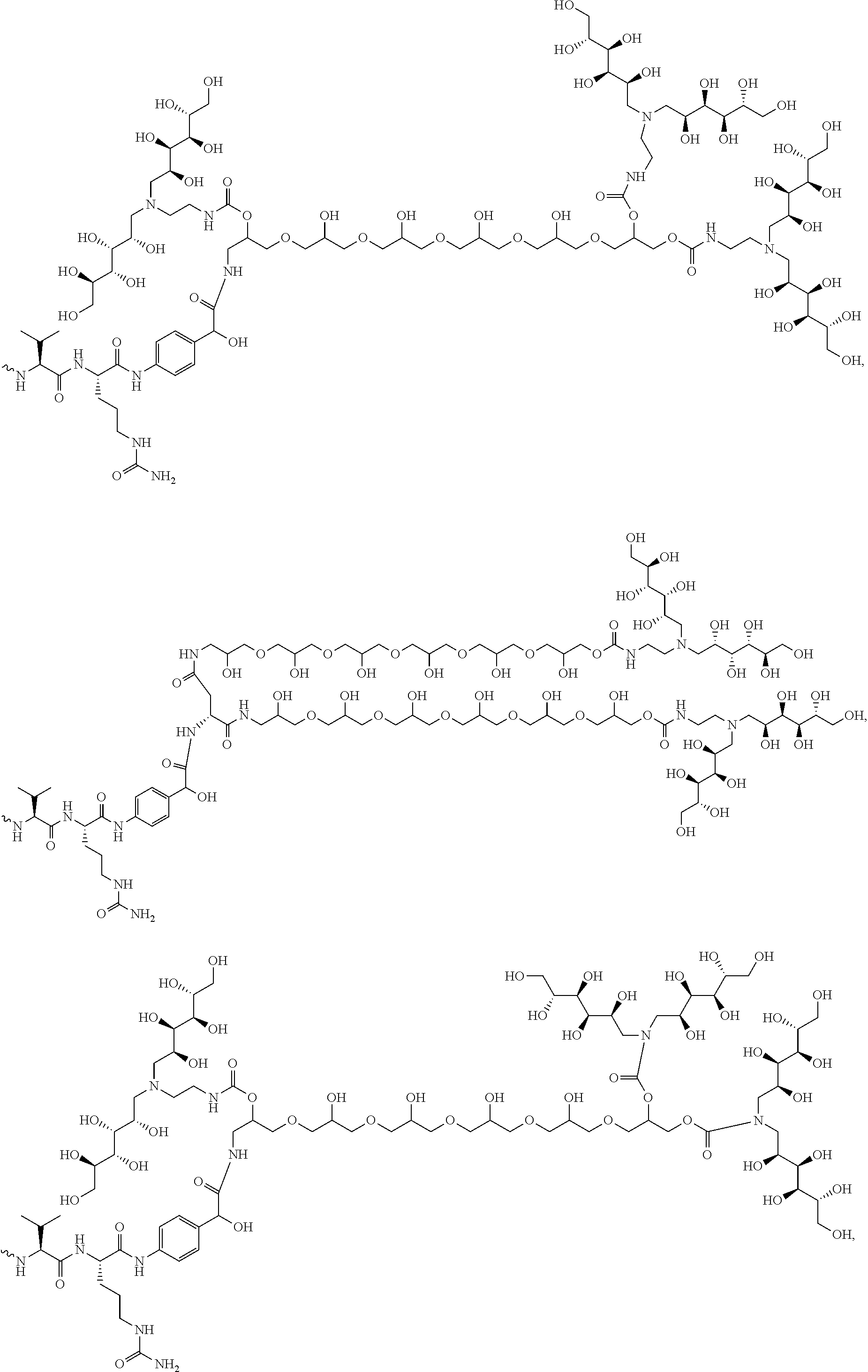 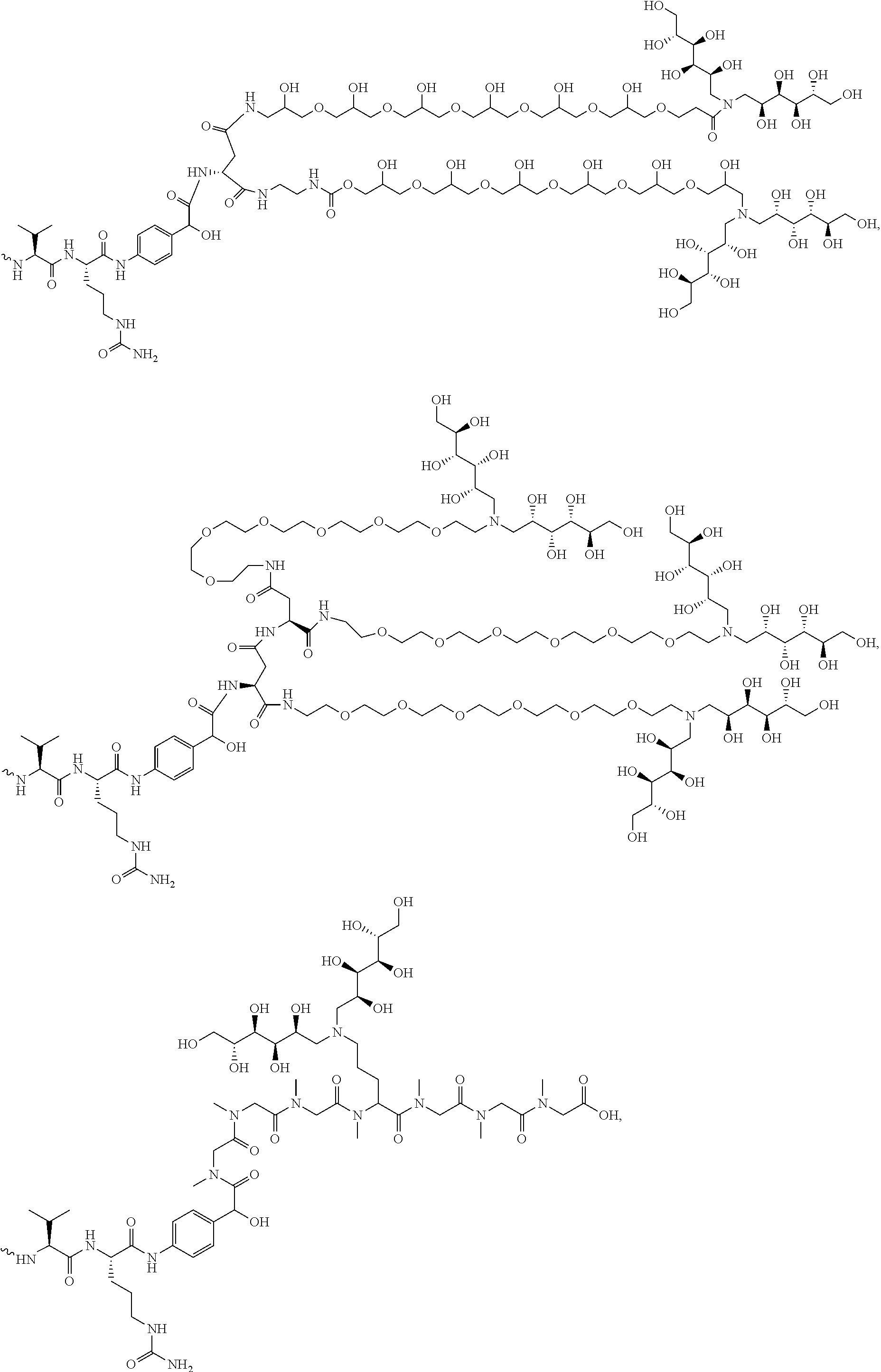 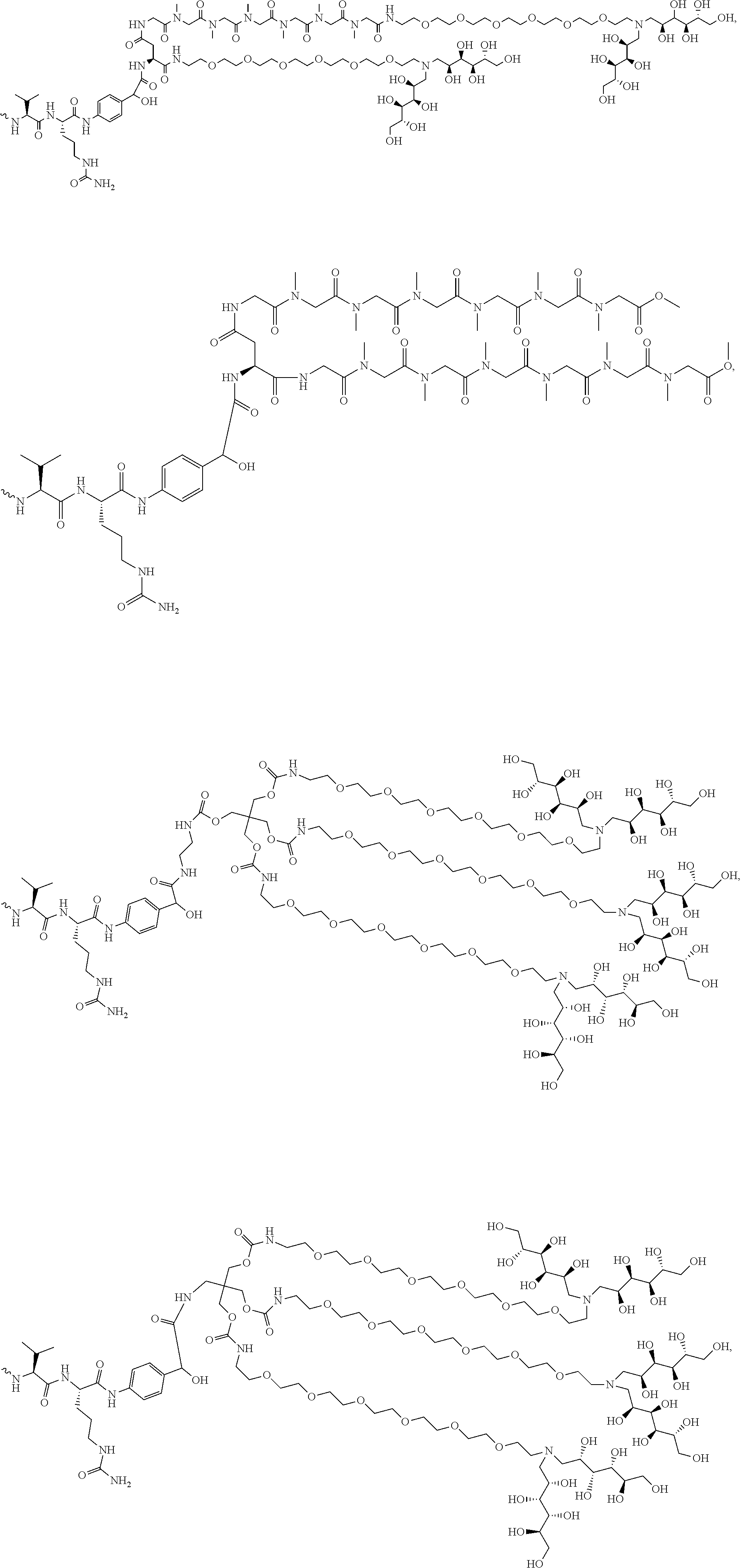 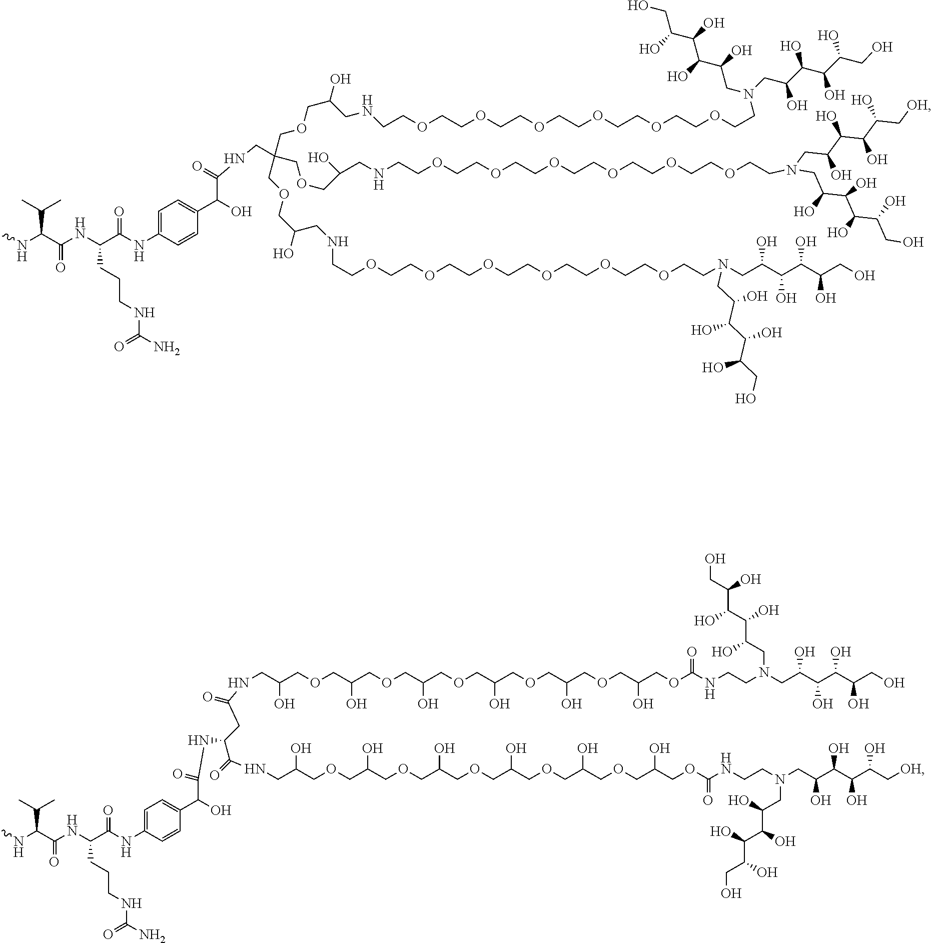 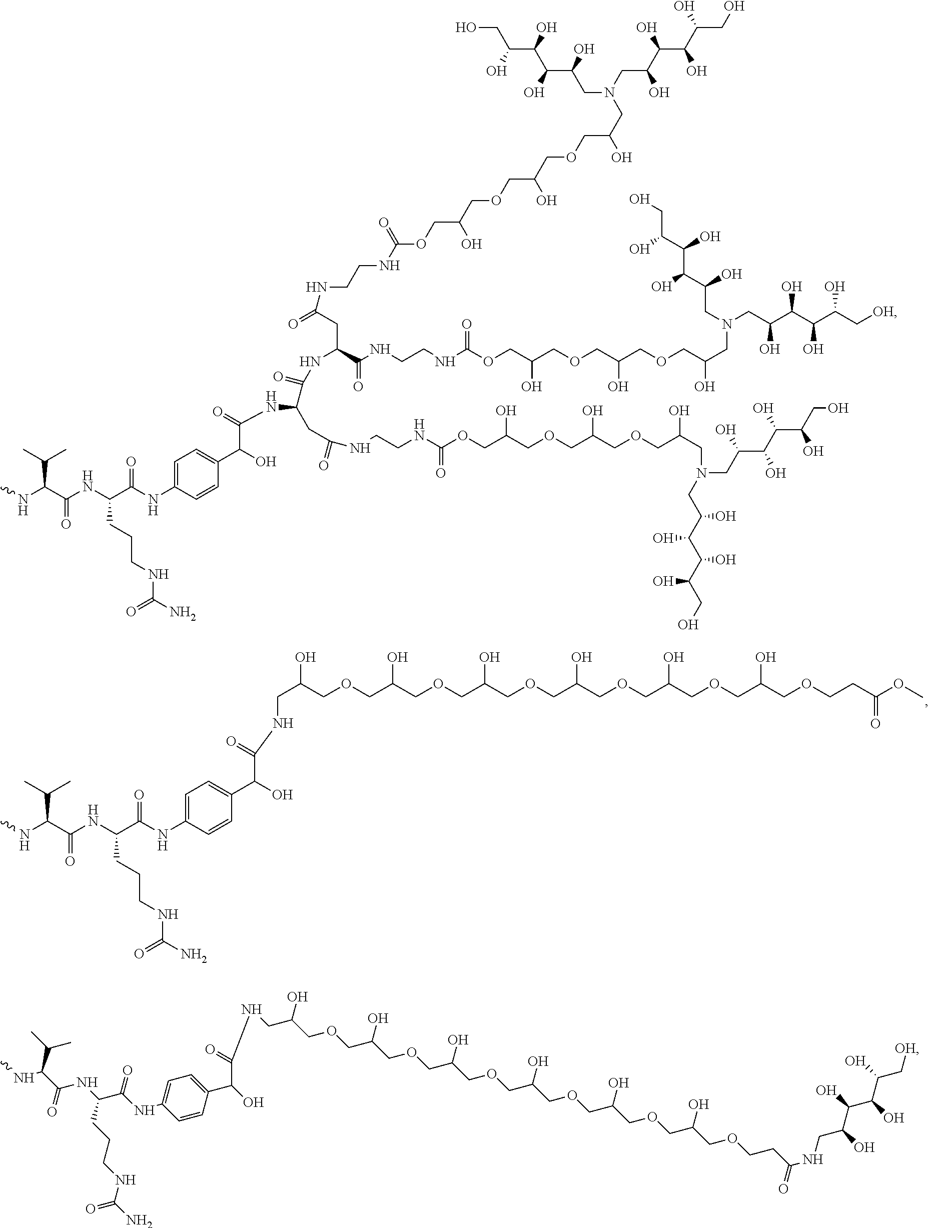 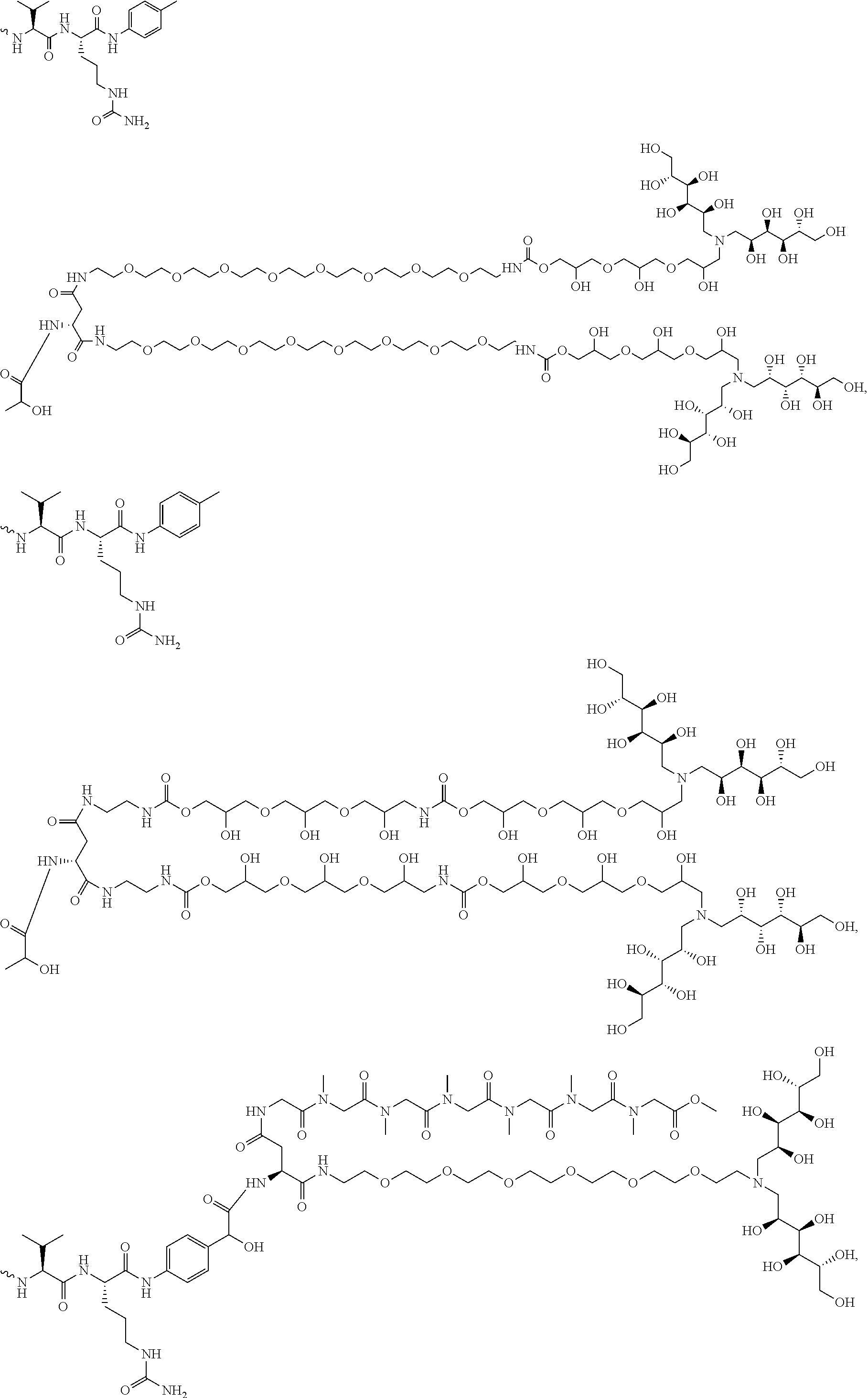 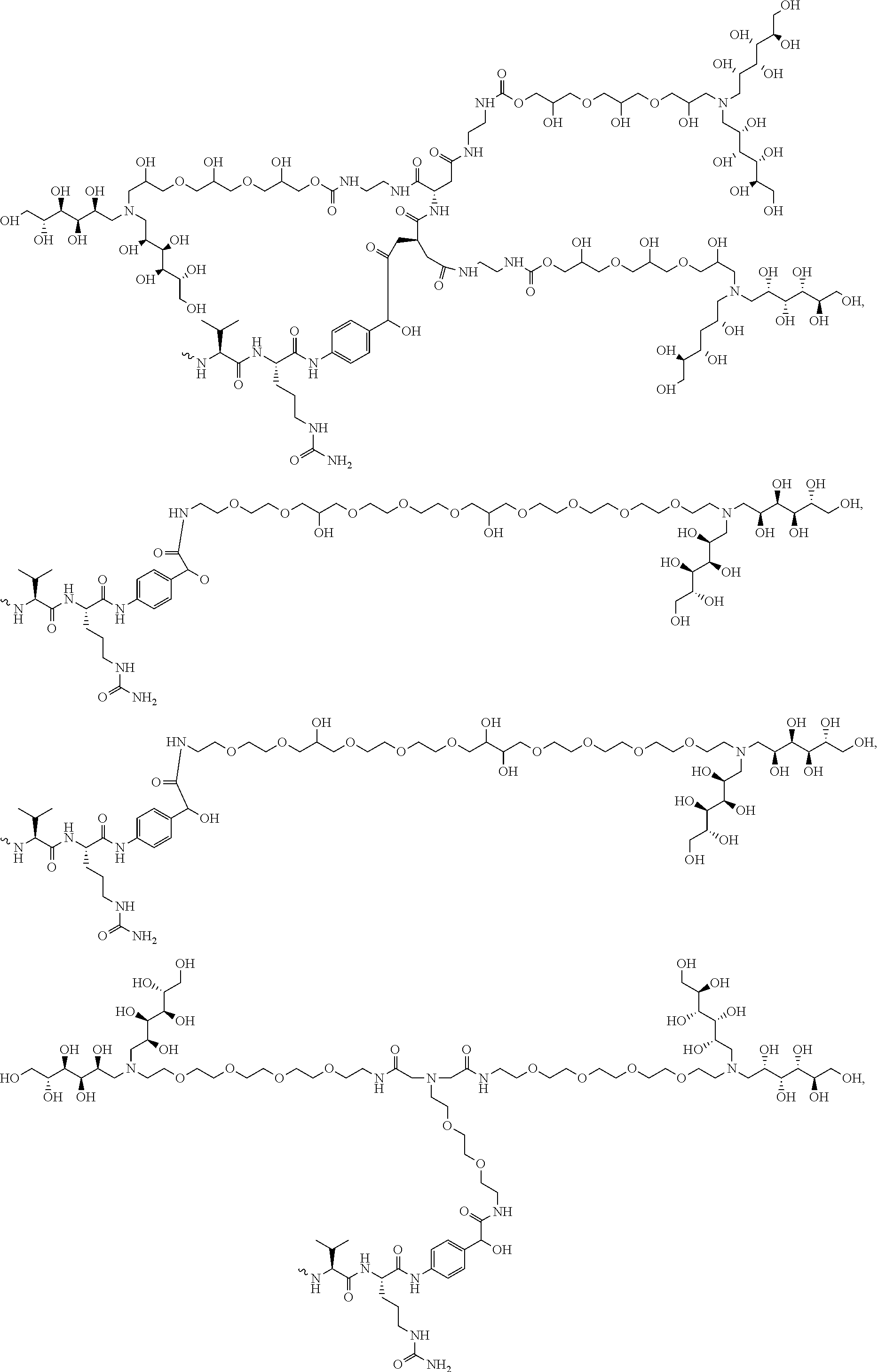 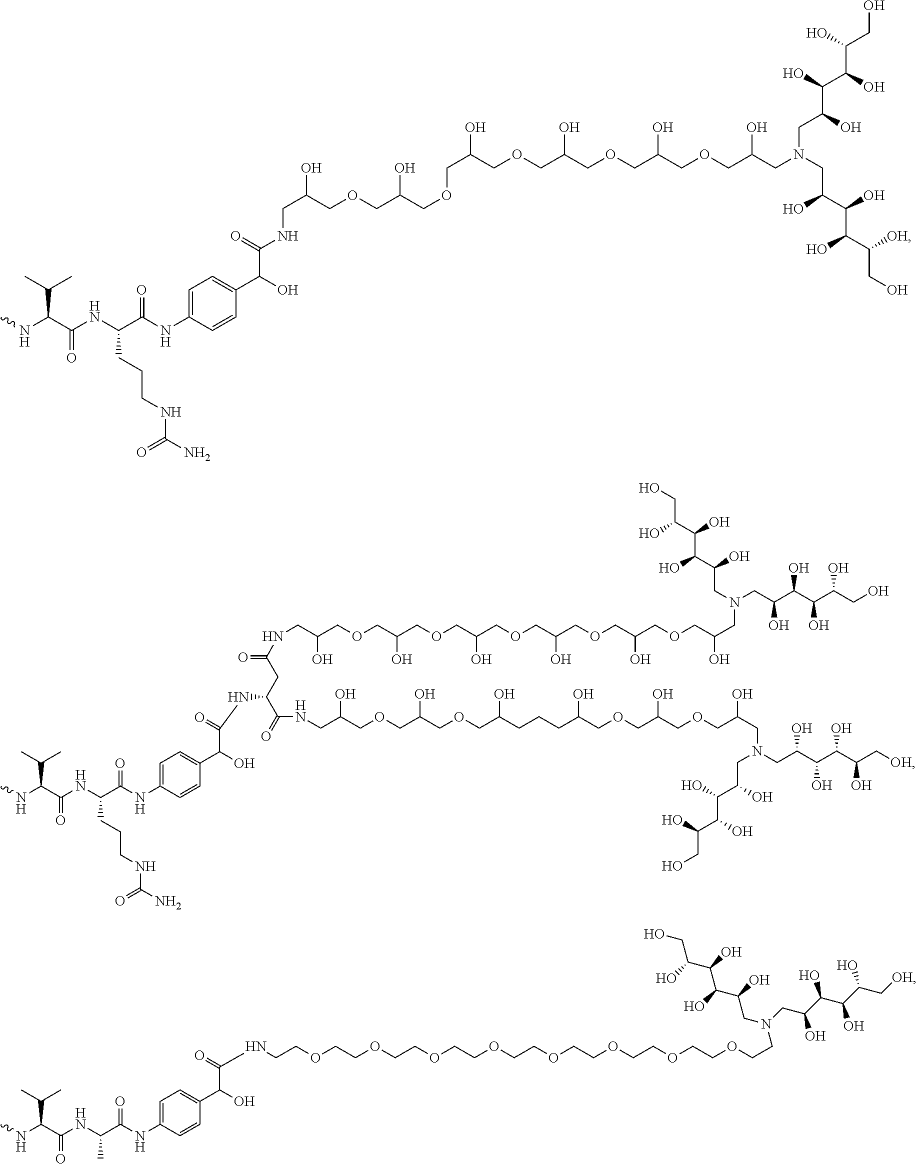 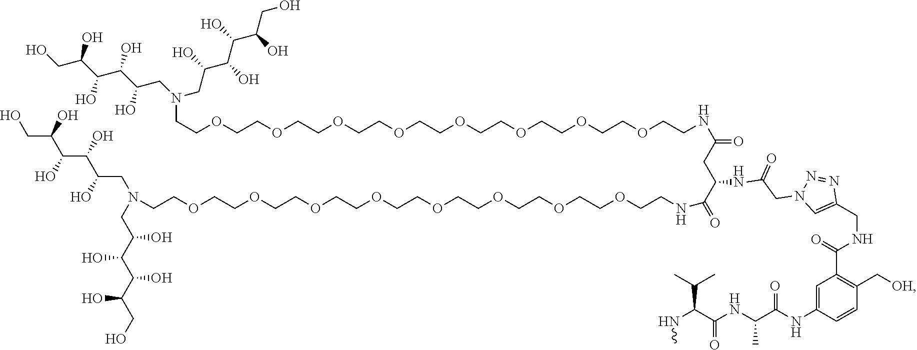
wherein the wavy line on the amino group indicates an attachment site to the Stretcher group; or, prior to attachment to the Stretcher group, indicates H; and the H on the benzylic OH is optionally replaced with a bond to at least one of the Drug units or a linking group attached to the at least one of the Drug units.

72. The Linker compound of any one of claims 1 to 69, wherein the enzyme-cleavable group is joined to the Stretcher group by a non-peptidic linking group.

73. The Linker compound of claim 72, wherein the non-peptidic linking group is selected from optionally-substituted C₁-C₁₀ alkylene, optionally-substituted C₂-C₁₀ alkenylene, optionally-substituted C₂-C₁₀ alkynylene, or optionally-substituted polyethylene glycol.

74. The Linker compound of any one of claims 1-73, comprising the Stretcher group attached to the enzyme-cleavable group.

75. The Linker compound of claim 74, wherein the Stretcher group is selected from the following: 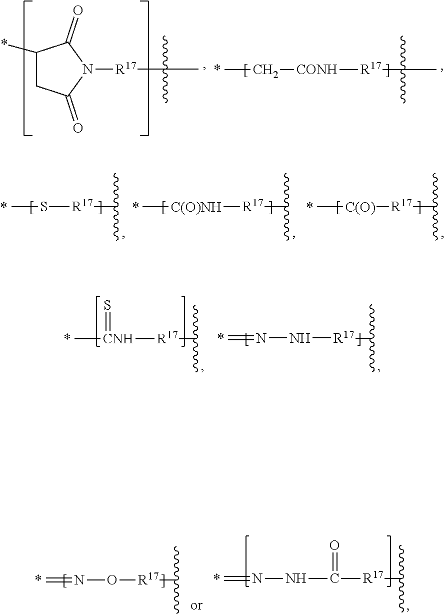
wherein R¹⁷ is —C₁-C₁₀ alkylene-, —C₁-C₁₀ heteroalkylene-, —C₃-C₈ carbocyclo-, —O—(C₁-C₈ alkylene)-, —(CH₂—O—CH₂)_{b}—C₁-C₈ alkylene- (where b is 1 to 26), —C₁-C₈ alkylene-(CH₂—O—CH₂)_{b}— (where b is 1 to 26), —C₁-C₈ alkylene-(CH₂—O—CH₂)_{b}—C₁-C₈ alkylene- (where b is 1 to 26), -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁(alkylene-(C₃-C₈ heterocyclo)-, —(C₃-C₈ heterocyclo)-C₁-C₁₀ alkylene-, —C₁-C₁₀ alkylene-C(═O)—, —C₁-C₁₀alkylene-C(O)NH—C₁-C₈alkylene-[O—CH₂—CH₂]ₙ—C(O)— (where n is 1 to 26), C₁-C₁₀ heteroalkylene-C(═O)—, —C₁-C₃ alkylene-(CH₂—O—CH₂)_{b}—C(═O)— (where b is 1 to 26), —(CH₂—O—CH₂)_{b}—C₁-C₈ alkylene-C(═O)— (where b is 1 to 26), —C₁-C₈ alkylene-(CH₂—O—CH₂)_{b}—C₁-C₈ alkylene-C(═O)— (where b is 1 to 26), —C₃-C₈ carbocyclo-C(═O)—, —O—(C₁-C₈ alkyl)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, —C₁-C₁₀ heteroalkylene-NH—, —C₁-C₈ alkylene-(CH₂—O—CH₂)_{b}—NH— (where b is 1 to 26), —(CH₂—O—CH₂)_{b}—C₁-C₈ alkylene-NH— (where b is 1 to 26), —C₁-C₈ alkylene-(CH₂—O—CH₂)_{b}—C₁-C₈ alkylene-NH— (where b is 1 to 26), —C₁-C₈ alkylene-(C(═O))—NH—(CH₂—O—CH₂)_{b}—C(═O)-(where b is 1 to 26), —C₁-C₈ alkylene-(C(═O))—NH—(CH₂—O—CH₂)_{b}—C₁-C₈ alkylene-C(═O)-(where b is 1 to 26), —C₁—C alkylene-NH—(C(═O))—(CH₂—O—CH₂)_{b}—NH— (where b is 1 to 26), —C₁-C₈ alkylene-NH—(C(═O))—(CH₂—O—CH₂)_{b}—C₁-C₈ alkylene-NH— (where b is 1 to 26), —C₃-C₈carbocyclo-NH—, —O—(C₁-C₈ alkyl)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₅ alkyl)-S—, -arylene-S—, —C₁-C₁(alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—; or
wherein the Stretcher group comprises maleimido(C₁-C₁₀ alkylene-C(O)—, maleimido(CH₂OCH₂)p₂(C₁-C₁₀ alkyene)C(O)—, maleimido(C₁-C₁₀alkyene) (CH₂OCH₂)ₚ₂C(O)—, or a ring open form thereof, wherein p2 is from 1 to 26;
and wherein * is an attachment site to the Targeting group, and the wavy line is an attachment site to the enzyme-cleavable group.

76. The Linker compound of claim 74, wherein the Stretcher group is selected from the following: 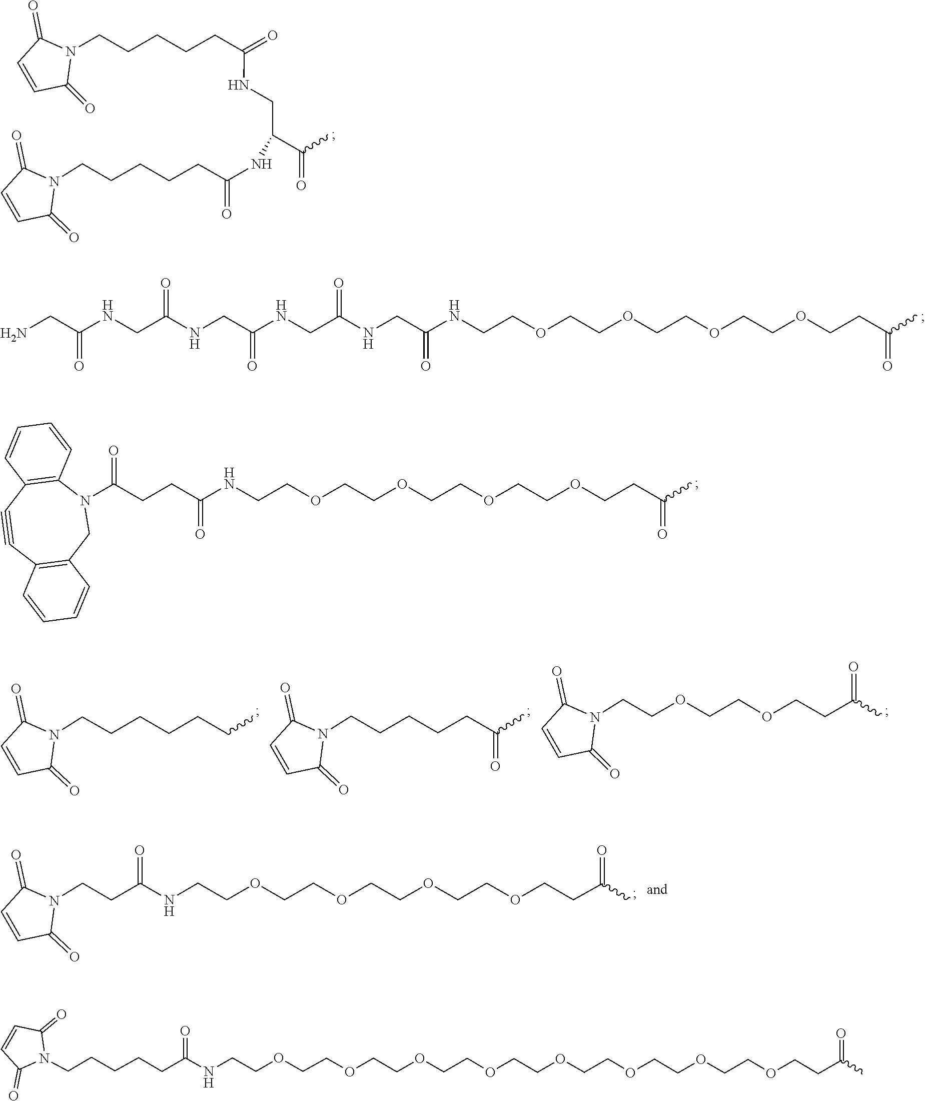
wherein the wavy line [symbol] indicates an attachment site of the Stretcher group to the enzyme-cleavable group, and the attachment site to the Targeting group is on the maleimide, primary amine or alkyne functional group.

77. The Linker compound of claim 1, having one of the following structures: 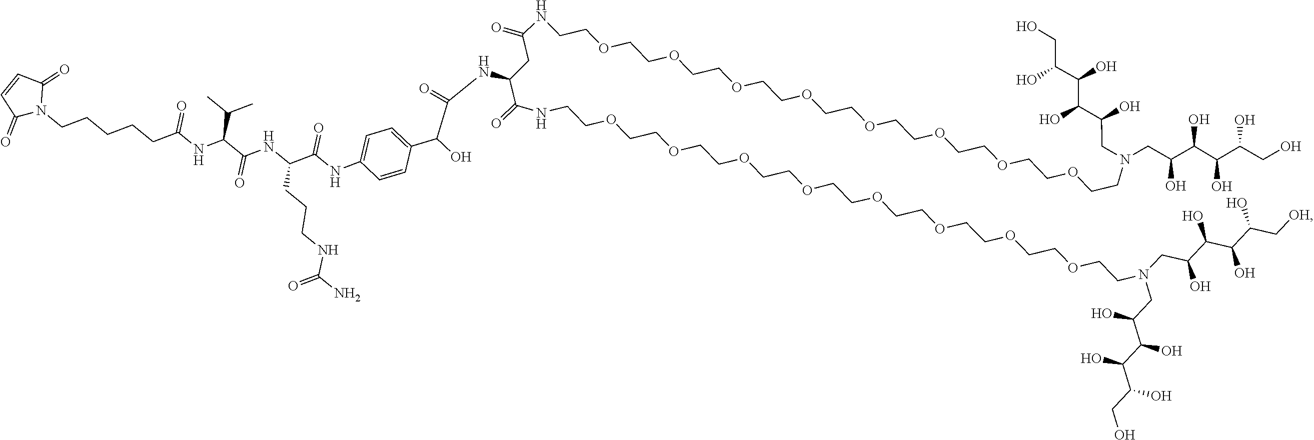 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@@H](CC(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1] 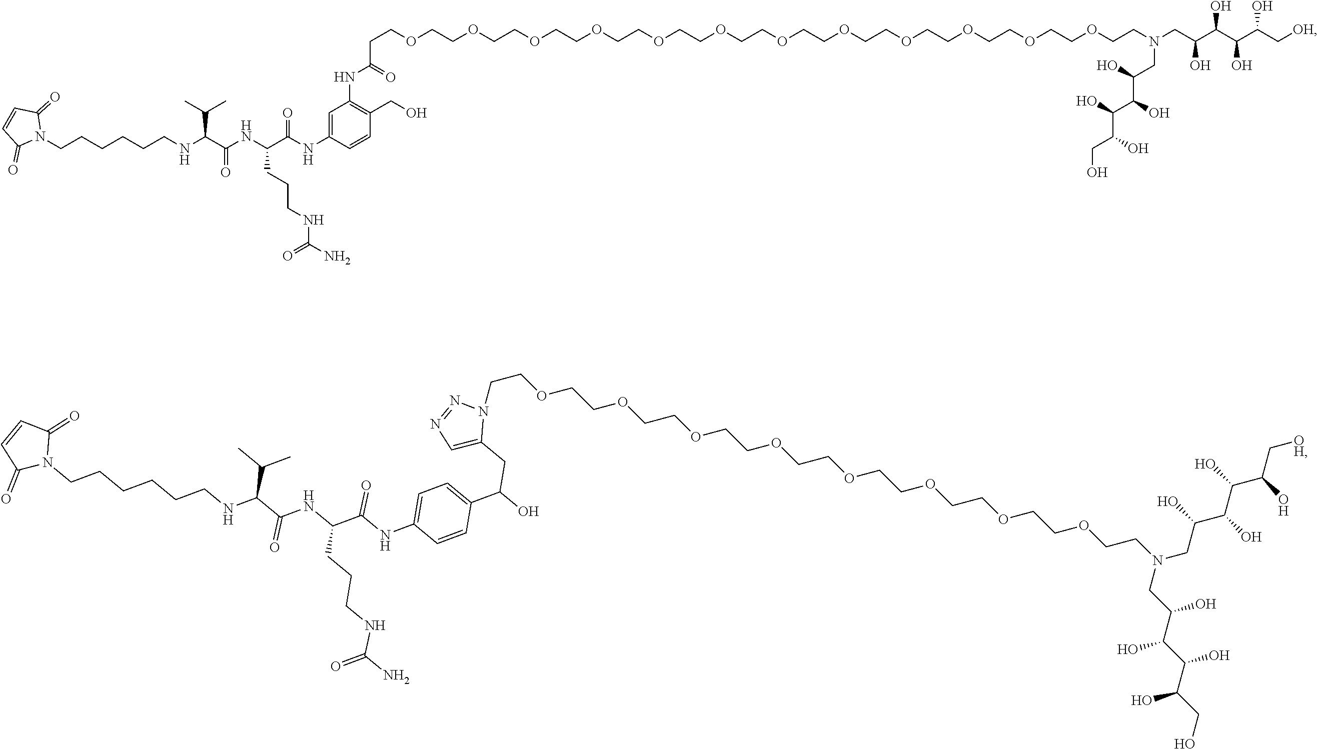 [SMILES: CC(C)[C@H](NCCCCCCN1C(=O)C=CC1=O)C(=O)N[C@@H](CCCNC(N)=O)C(=O)Nc1ccc(CO)c(NC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1 ; CC(C)[C@H](NCCCCCCN1C(=O)C=CC1=O)C(=O)N[C@@H](CCCNC(N)=O)C(=O)Nc1ccc(C(O)Cc2cnnn2CCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1] 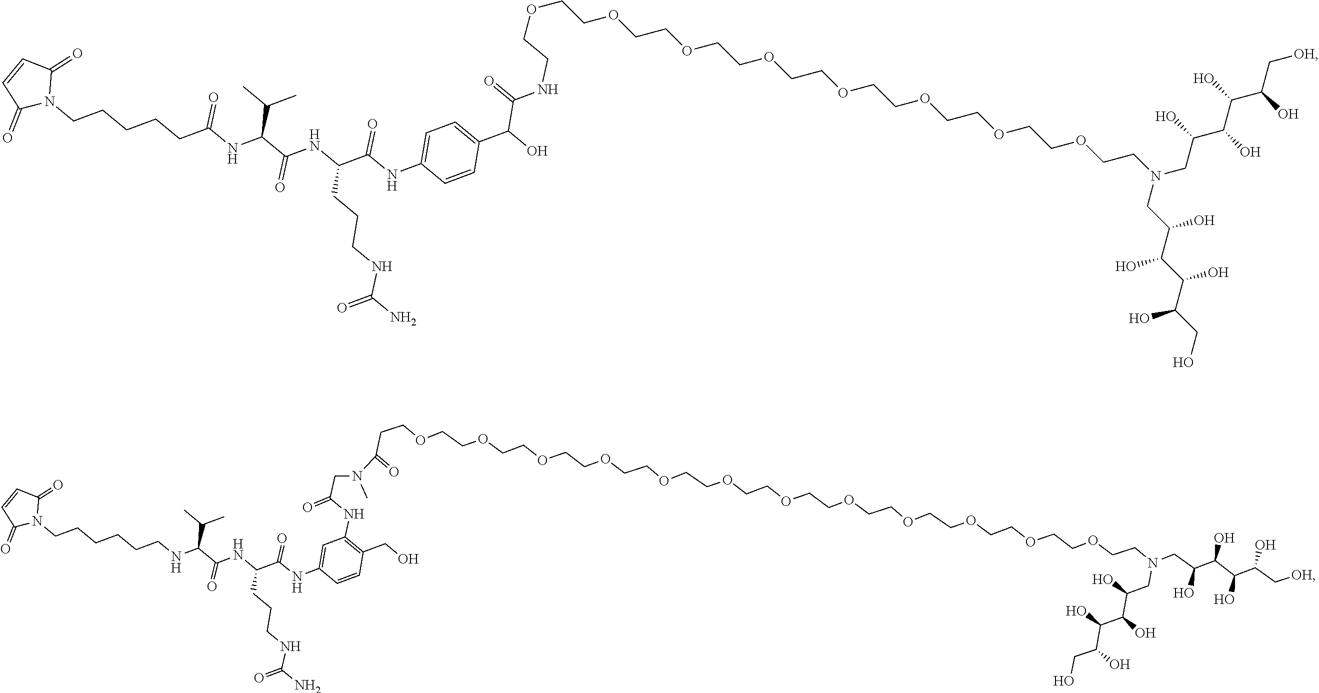 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1 ; CC(C)[C@H](NCCCCCCN1C(=O)C=CC1=O)C(=O)N[C@@H](CCCNC(N)=O)C(=O)Nc1ccc(CO)c(NC(=O)CN(C)C(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1] 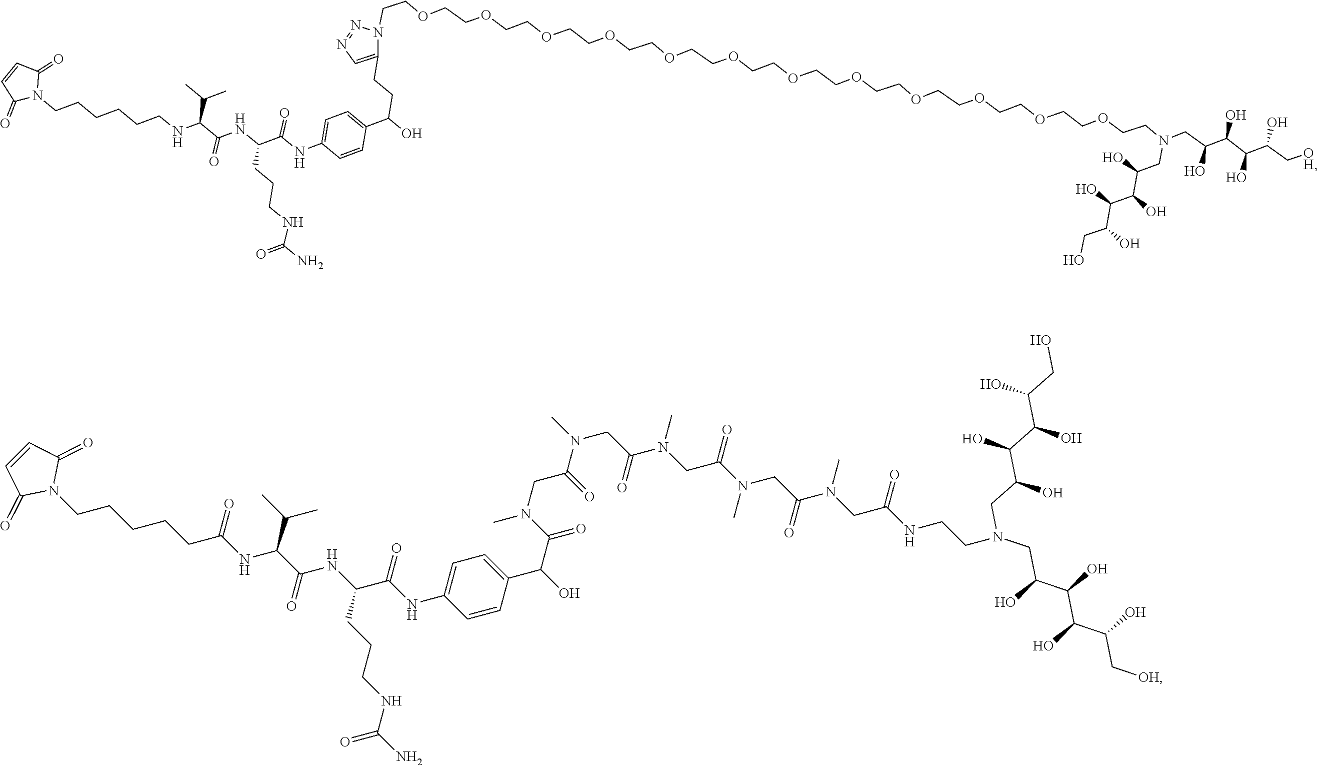 [SMILES: CC(C)[C@H](NCCCCCCN1C(=O)C=CC1=O)C(=O)N[C@@H](CCCNC(N)=O)C(=O)Nc1ccc(C(O)CCc2cnnn2CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1 ; CC(C)[C@H](NC(=O)CCCCCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)cc1] 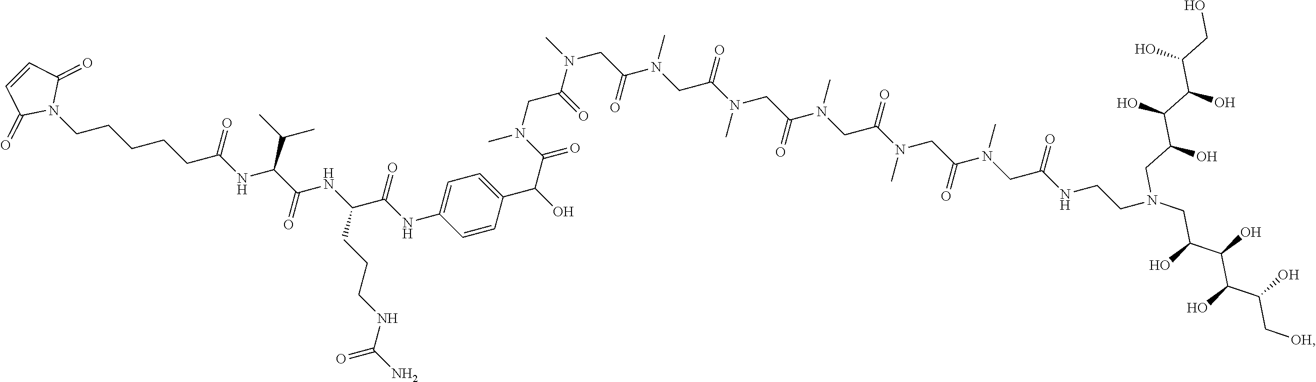 [SMILES: CC(C)[C@H](NC(=O)CCCCCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)CC(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)cc1] 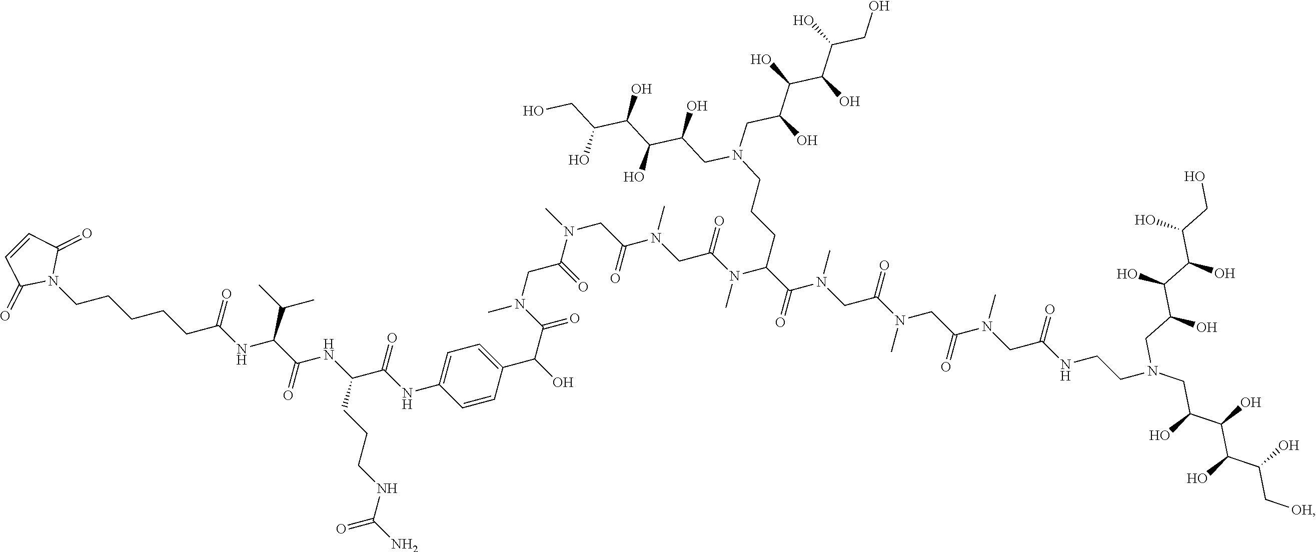 [SMILES: CC(C)[C@H](NC(=O)CCCCCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)cc1] 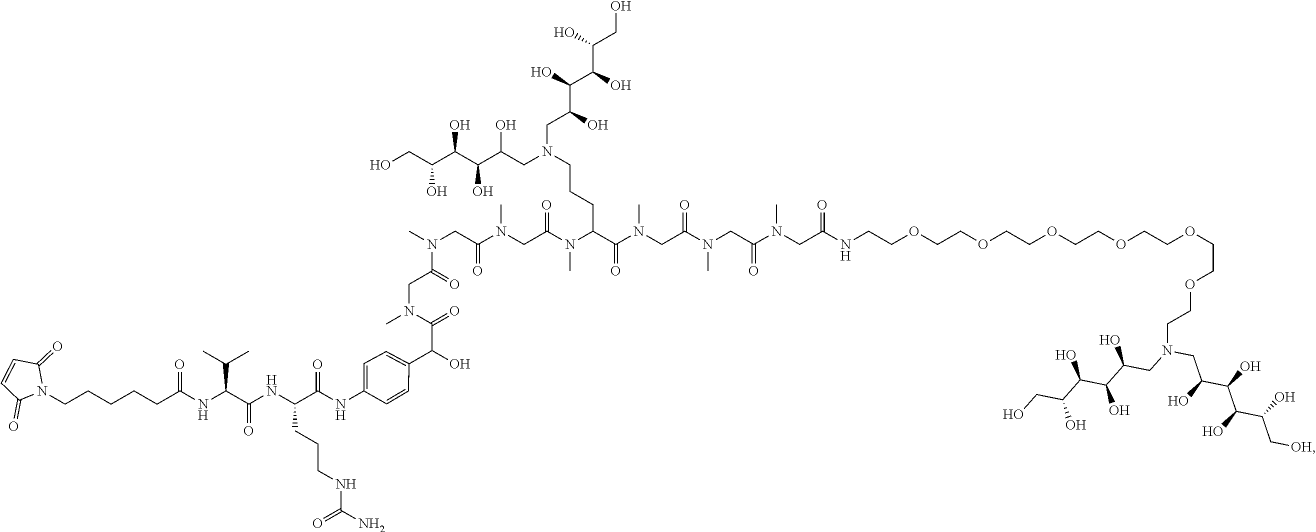 [SMILES: CC(C)[C@H](NC(=O)CCCCCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)C(CCCN(CC(O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1] 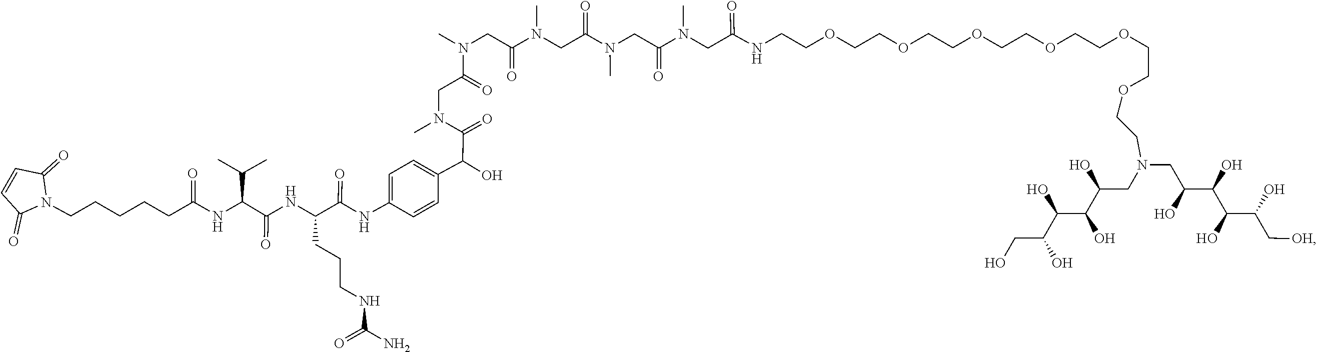 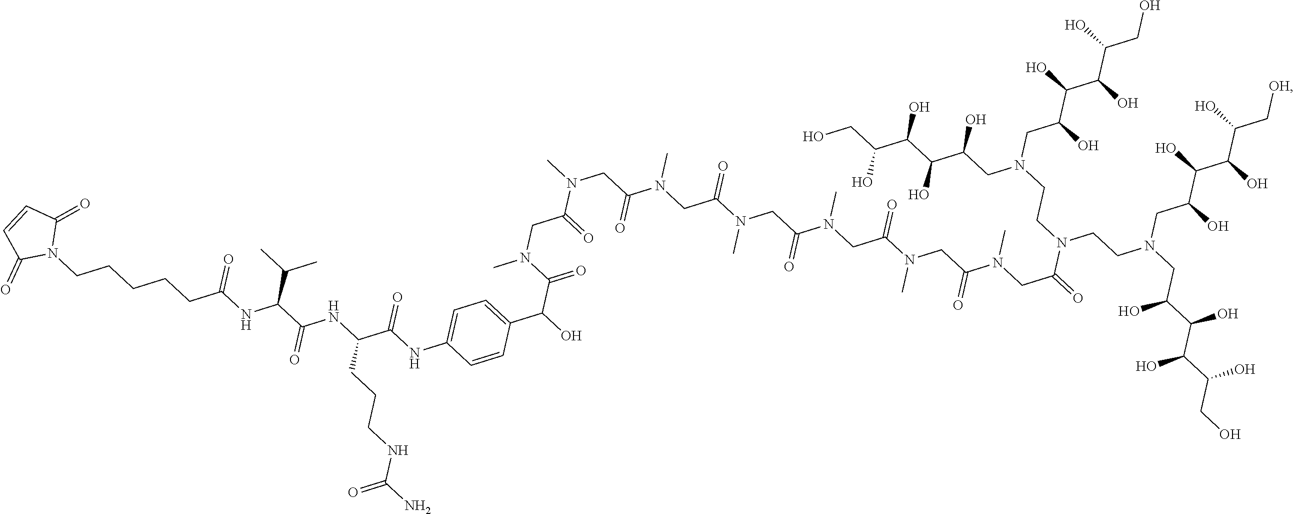 [SMILES: CC(C)[C@H](NC(=O)CCCCCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)CC(=O)N(C)CC(=O)N(C)CC(=O)N(C)CC(=O)N(CCN(C[C@H](O)[C@@H](O)[C@H](O)[C@H](O)CO)C[C@H](O)[C@@H](O)[C@H](O)[C@H](O)CO)CCN(C[C@H](O)[C@@H](O)[C@H](O)[C@H](O)CO)C[C@H](O)[C@@H](O)[C@H](O)[C@H](O)CO)cc1] 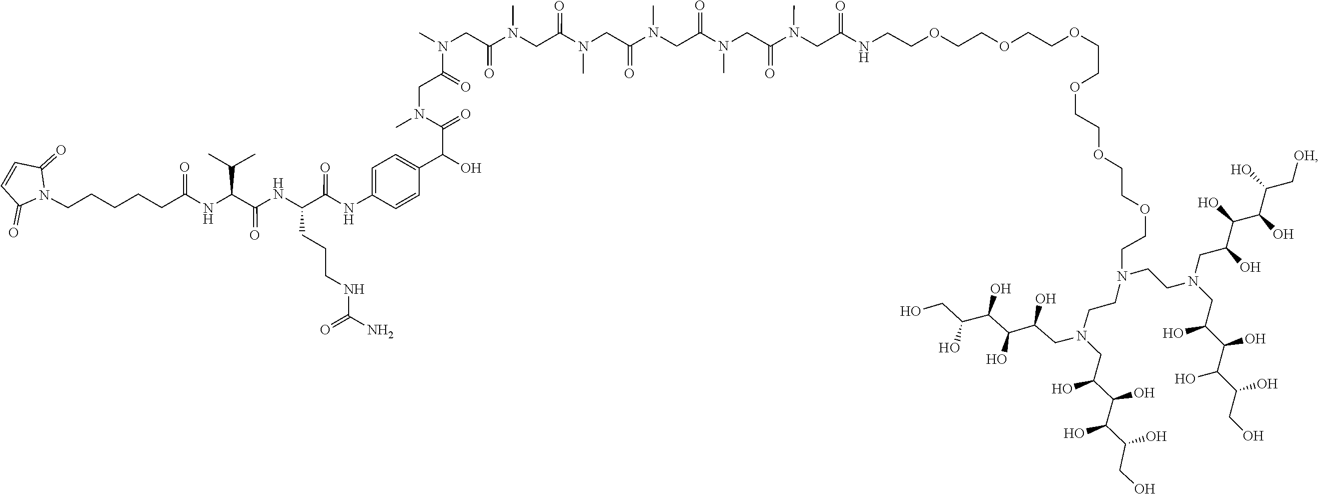 [SMILES: CC(C)[C@H](NC(=O)CCCCCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)CC(=O)N(C)CC(=O)N(C)CC(=O)N(C)CC(=O)NCCOCCOCCOCCOCCOCCOCCN(CCN(C[C@H](O)[C@@H](O)[C@H](O)[C@H](O)CO)C[C@H](O)[C@@H](O)[C@H](O)[C@H](O)CO)CCN(C[C@H](O)[C@@H](O)[C@H](O)[C@H](O)CO)C[C@H](O)[C@@H](O)C(O)[C@H](O)CO)cc1] 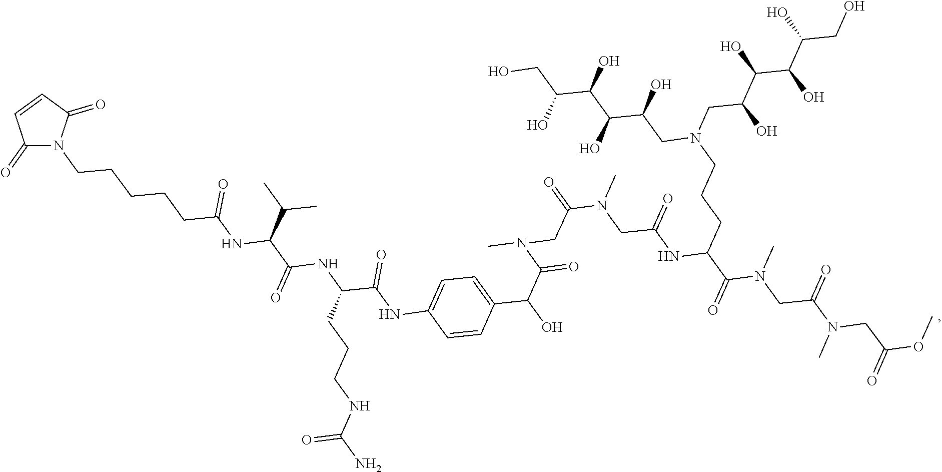 [SMILES: COC(=O)CN(C)C(=O)CN(C)C(=O)C(CCCN(C[C@H](O)[C@@H](O)[C@H](O)[C@H](O)CO)C[C@H](O)[C@@H](O)[C@H](O)[C@H](O)CO)NC(=O)CN(C)C(=O)CN(C)C(=O)C(O)c1ccc(NC(=O)[C@H](CCCNC(N)=O)NC(=O)[C@@H](NC(=O)CCCCCN2C(=O)C=CC2=O)C(C)C)cc1] 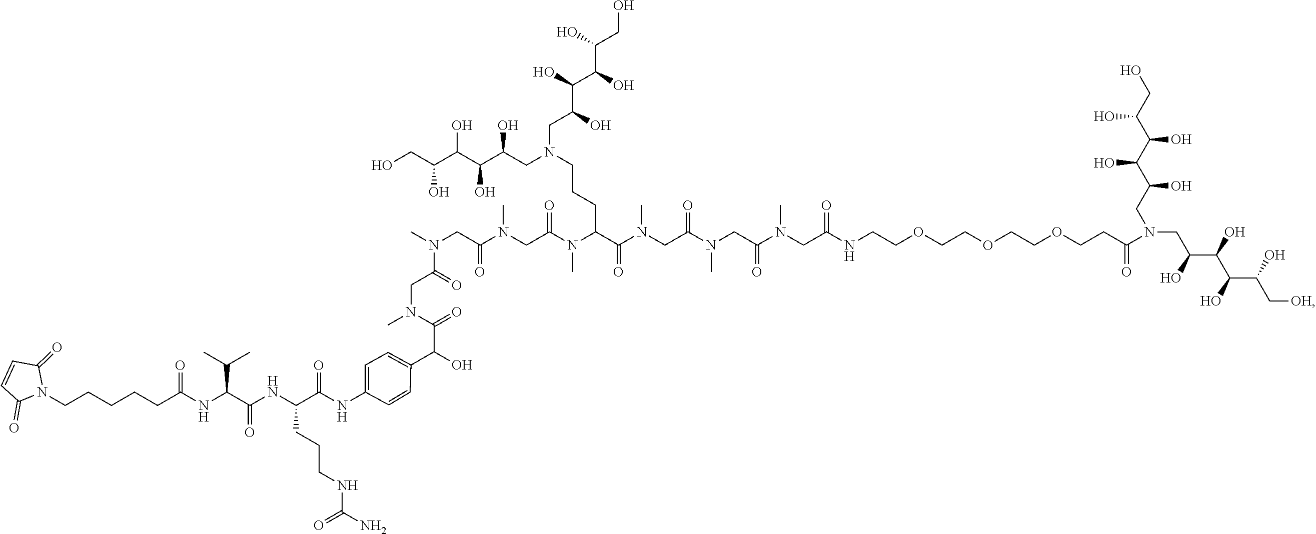 [SMILES: CC(C)[C@H](NC(=O)CCCCCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)C(O)[C@H](O)CO)C(=O)N(C)CC(=O)N(C)CC(=O)N(C)CC(=O)NCCOCCOCCOCCC(=O)N(C[C@H](O)[C@@H](O)[C@H](O)[C@H](O)CO)C[C@H](O)[C@@H](O)[C@H](O)[C@H](O)CO)cc1] 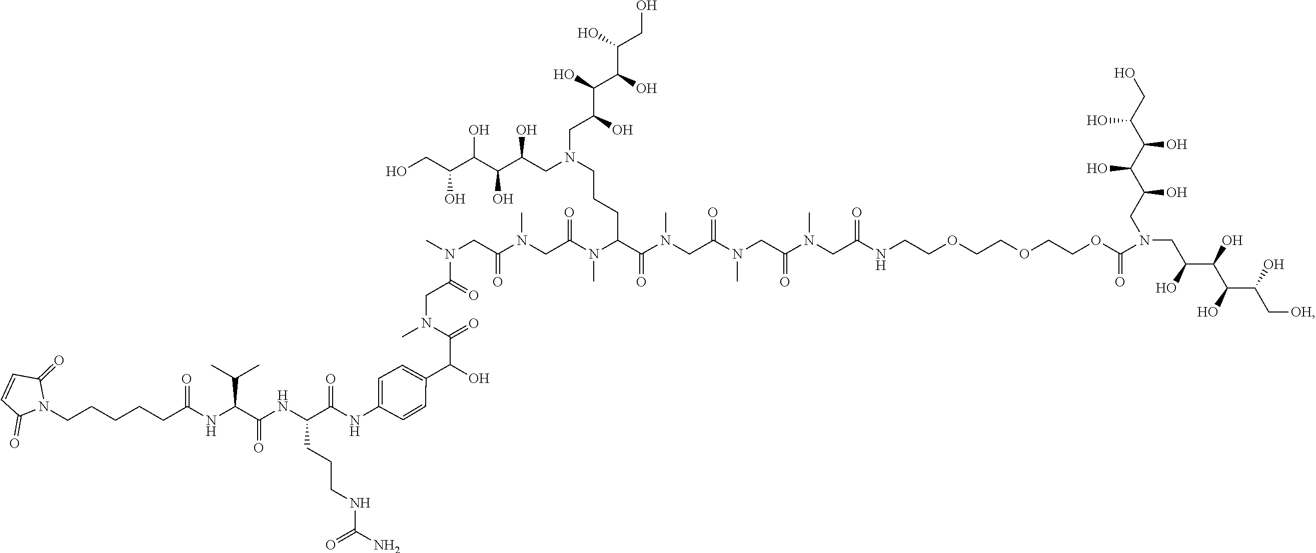 [SMILES: CC(C)[C@H](NC(=O)CCCCCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)C(O)[C@H](O)CO)C(=O)N(C)CC(=O)N(C)CC(=O)N(C)CC(=O)NCCOCCOCCOC(=O)N(C[C@H](O)[C@@H](O)[C@H](O)[C@H](O)CO)C[C@H](O)[C@@H](O)[C@H](O)[C@H](O)CO)cc1] 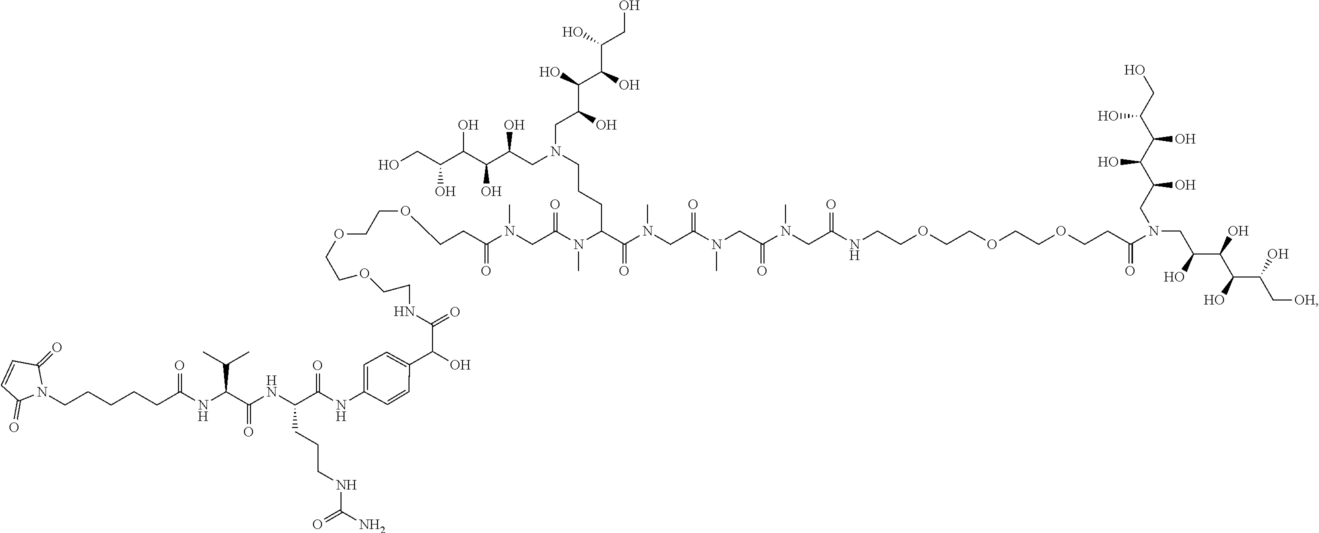 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCCOCCOCCOCCC(=O)N(C)CC(=O)N(C)C(CCCN(C[C@H](O)[C@@H](O)[C@H](O)[C@H](O)CO)C[C@H](O)[C@@H](O)C(O)[C@H](O)CO)C(=O)N(C)CC(=O)N(C)CC(=O)N(C)CC(=O)NCCOCCOCCOCCC(=O)N(C[C@H](O)[C@@H](O)[C@H](O)[C@H](O)CO)C[C@H](O)[C@@H](O)[C@H](O)[C@H](O)CO)cc1] 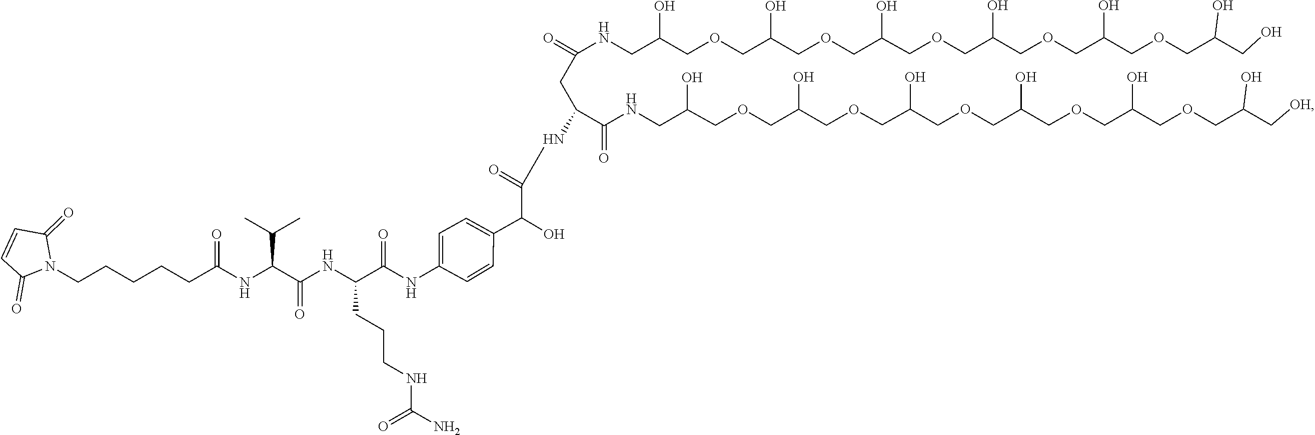 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCC(O)COCC(O)COCC(O)COCC(O)COCC(O)COCC(O)CO)C(=O)NCC(O)COCC(O)COCC(O)COCC(O)COCC(O)COCC(O)CO)cc1] 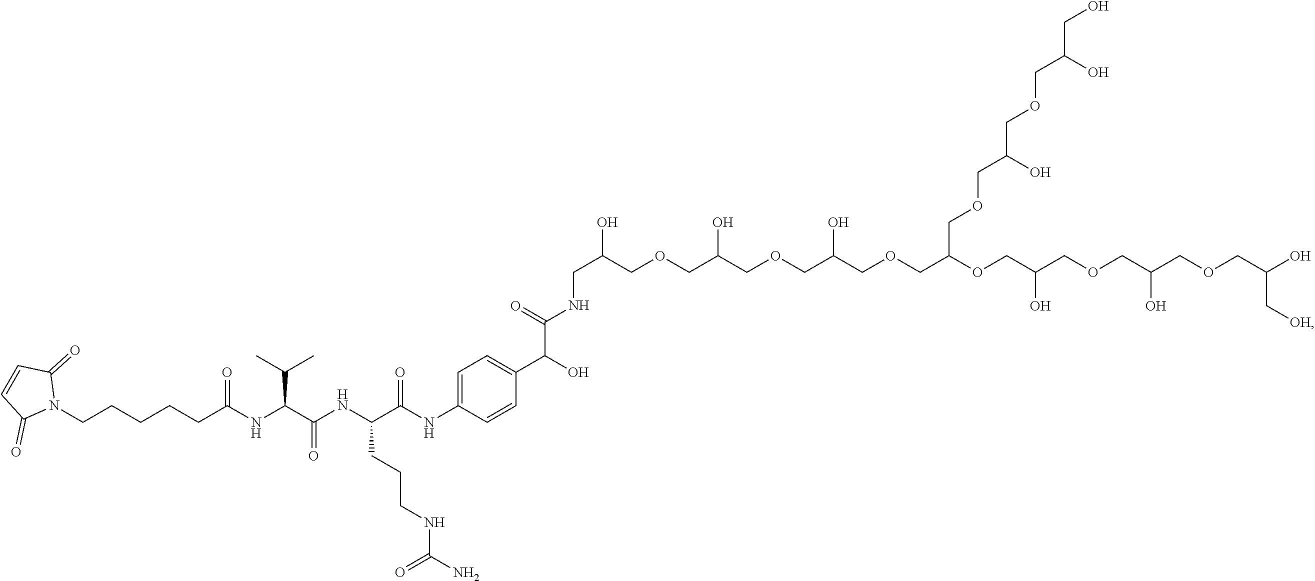 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCC(O)COCC(O)COCC(O)COCC(COCC(O)COCC(O)CO)OCC(O)COCC(O)COCC(O)CO)cc1] 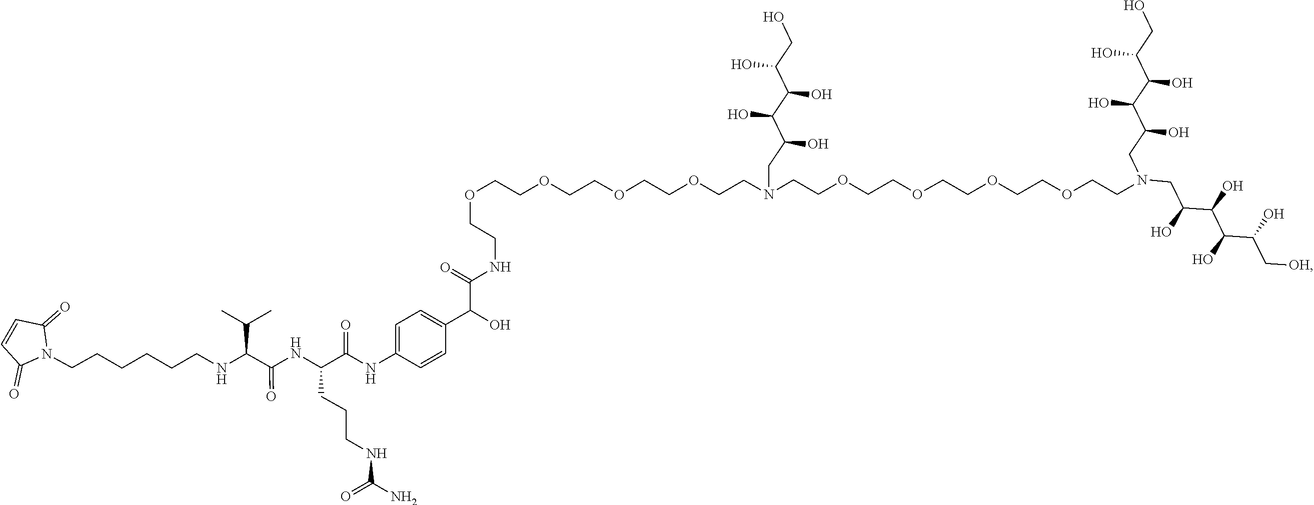 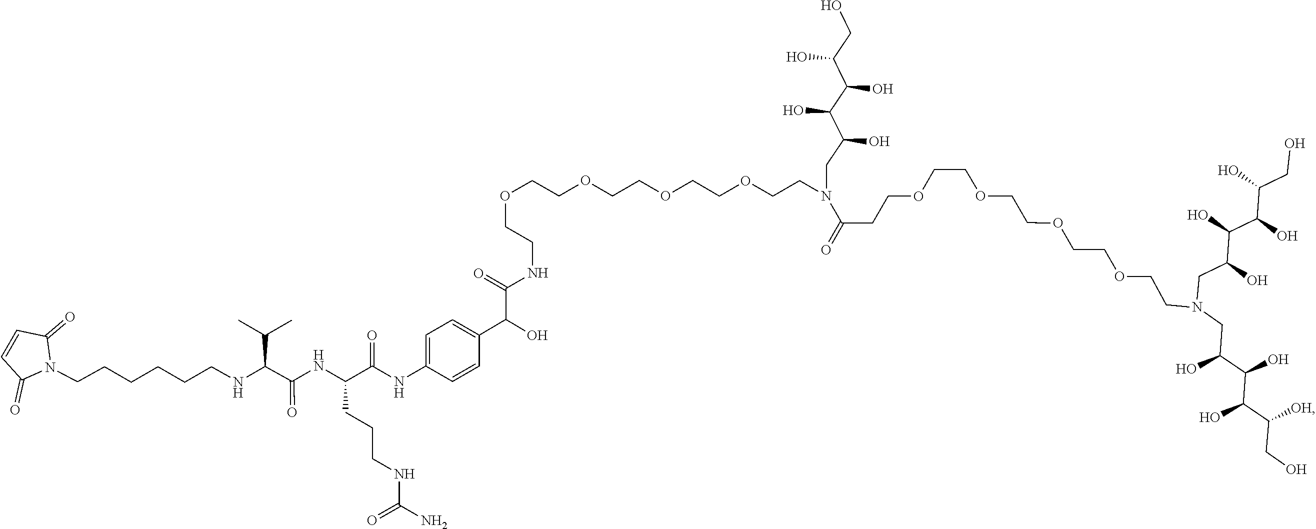 [SMILES: CC(C)[C@H](NCCCCCCN1C(=O)C=CC1=O)C(=O)N[C@@H](CCCNC(N)=O)C(=O)Nc1ccc(C(O)C(=O)NCCOCCOCCOCCOCCN(C[C@H](O)[C@@H](O)[C@H](O)[C@H](O)CO)C(=O)CCOCCOCCOCCOCCN(C[C@H](O)[C@@H](O)[C@H](O)[C@H](O)CO)C[C@H](O)[C@@H](O)[C@H](O)[C@H](O)CO)cc1] 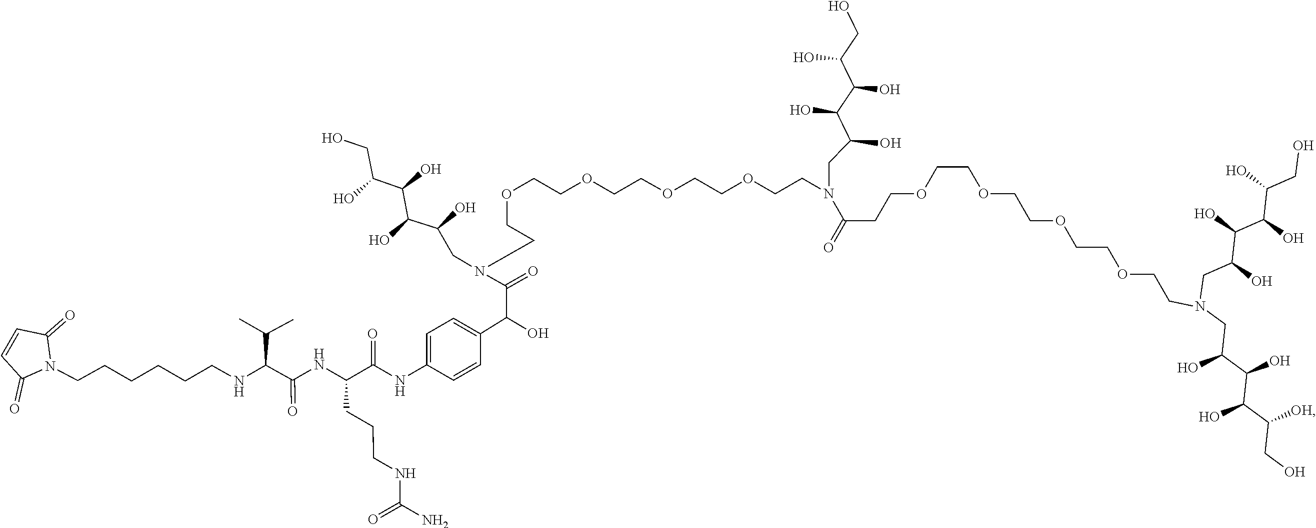 [SMILES: CC(C)[C@H](NCCCCCCN1C(=O)C=CC1=O)C(=O)N[C@@H](CCCNC(N)=O)C(=O)Nc1ccc(C(O)C(=O)N(CCOCCOCCOCCOCCN(C[C@H](O)[C@@H](O)[C@H](O)[C@H](O)CO)C(=O)CCOCCOCCOCCOCCN(C[C@H](O)[C@@H](O)[C@H](O)[C@H](O)CO)C[C@H](O)[C@@H](O)[C@H](O)[C@H](O)CO)C[C@H](O)[C@@H](O)[C@H](O)[C@H](O)CO)cc1] 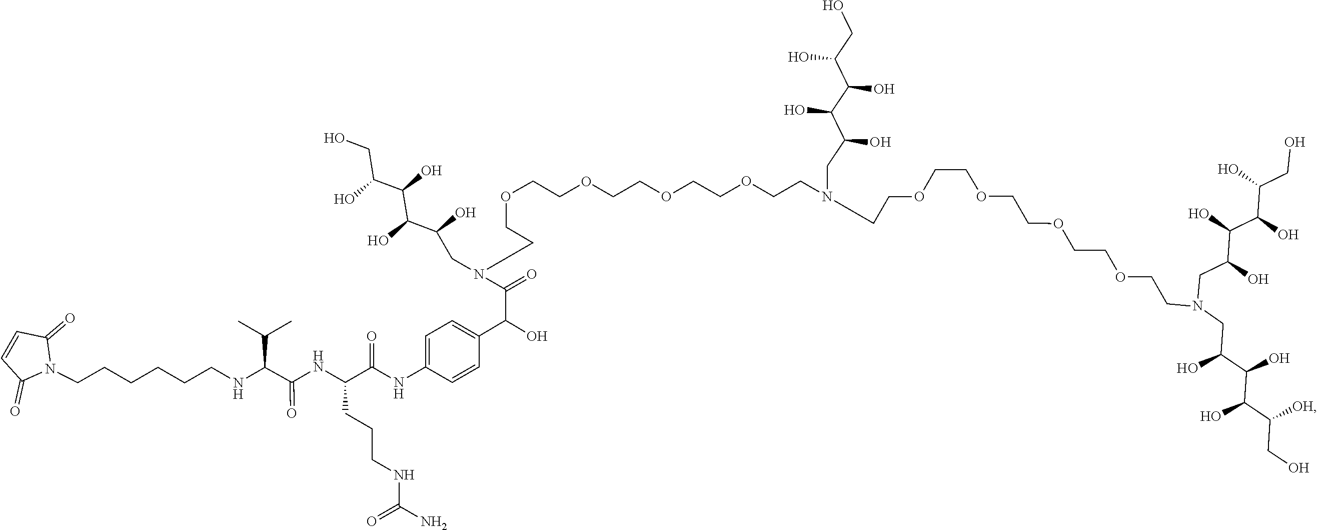 [SMILES: CC(C)[C@H](NCCCCCCN1C(=O)C=CC1=O)C(=O)N[C@@H](CCCNC(N)=O)C(=O)Nc1ccc(C(O)C(=O)N(CCOCCOCCOCCOCCN(CCOCCOCCOCCOCCN(C[C@H](O)[C@@H](O)[C@H](O)[C@H](O)CO)C[C@H](O)[C@@H](O)[C@H](O)[C@H](O)CO)C[C@H](O)[C@@H](O)[C@H](O)[C@H](O)CO)C[C@H](O)[C@@H](O)[C@H](O)[C@H](O)CO)cc1] 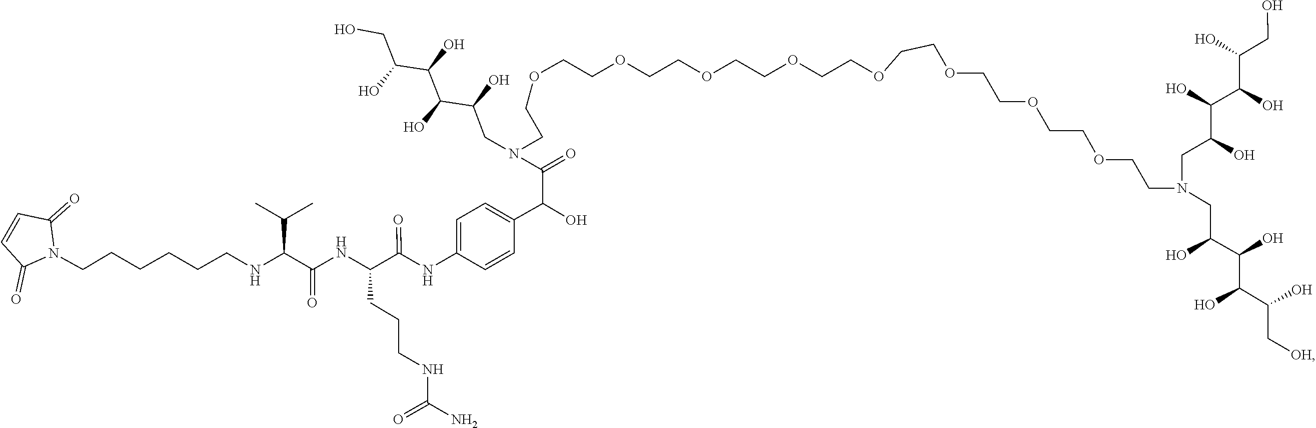 [SMILES: CC(C)[C@H](NCCCCCCN1C(=O)C=CC1=O)C(=O)N[C@@H](CCCNC(N)=O)C(=O)Nc1ccc(C(O)C(=O)N(CCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C[C@H](O)[C@@H](O)[C@H](O)[C@H](O)CO)cc1] 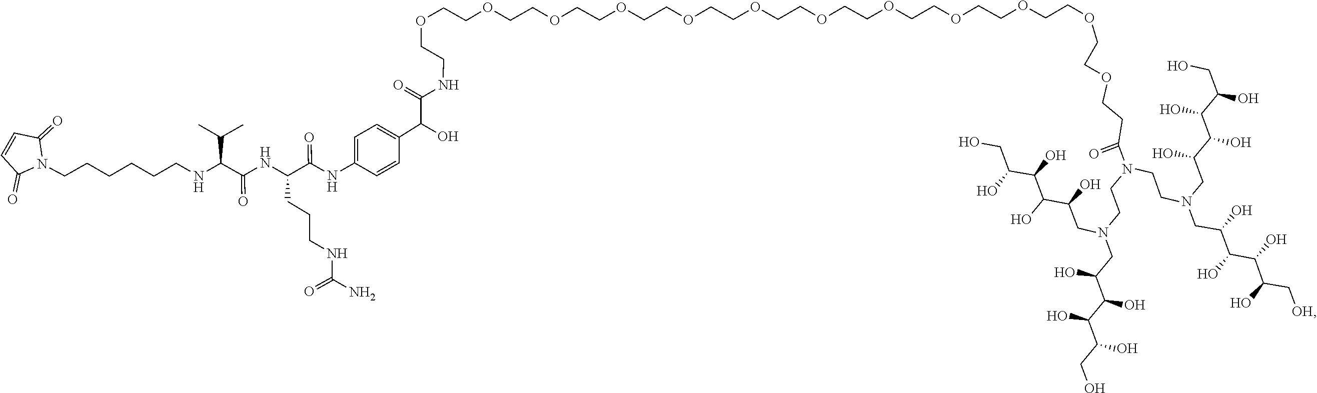 [SMILES: CC(C)[C@H](NCCCCCCN1C(=O)C=CC1=O)C(=O)N[C@@H](CCCNC(N)=O)C(=O)Nc1ccc(C(O)C(=O)NCCOCCOCCOCCOCCOCCOCCOCCOCCOCCOCCOCCOCCC(=O)N(CCN(C[C@H](O)C(O)[C@H](O)[C@H](O)CO)C[C@H](O)[C@@H](O)[C@H](O)[C@H](O)CO)CCN(C[C@H](O)[C@@H](O)[C@H](O)[C@H](O)CO)C[C@H](O)[C@@H](O)[C@H](O)[C@H](O)CO)cc1] 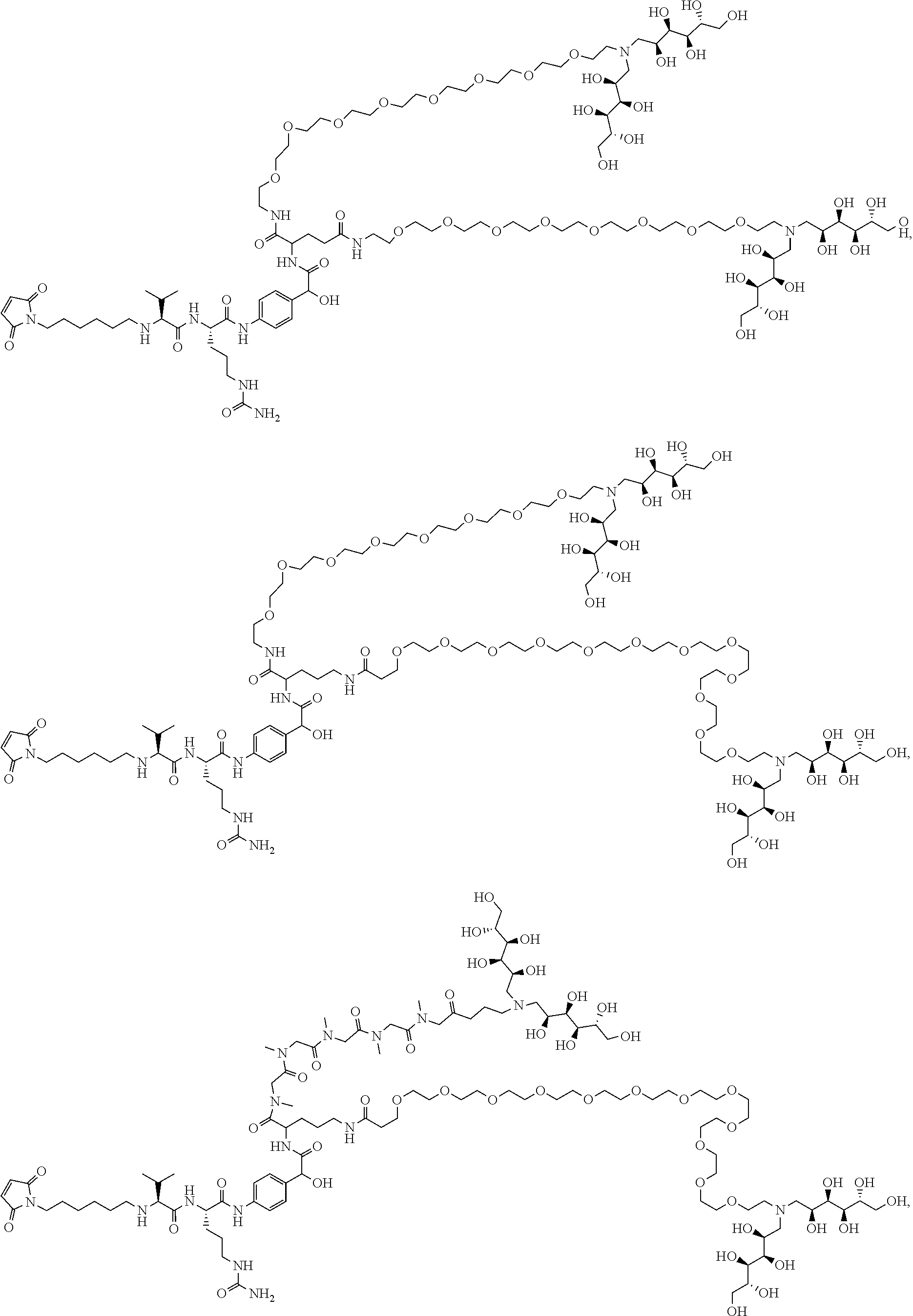 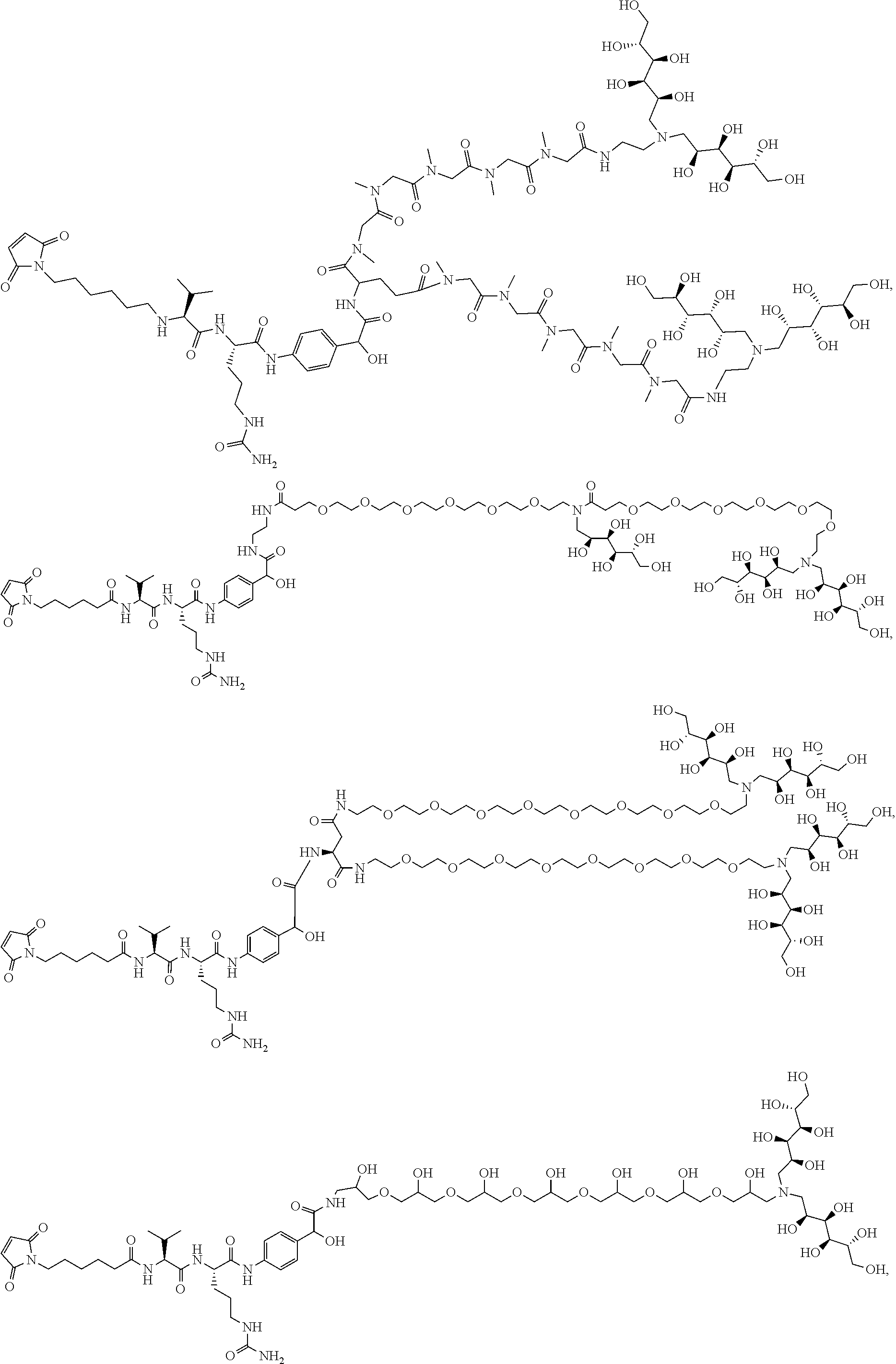 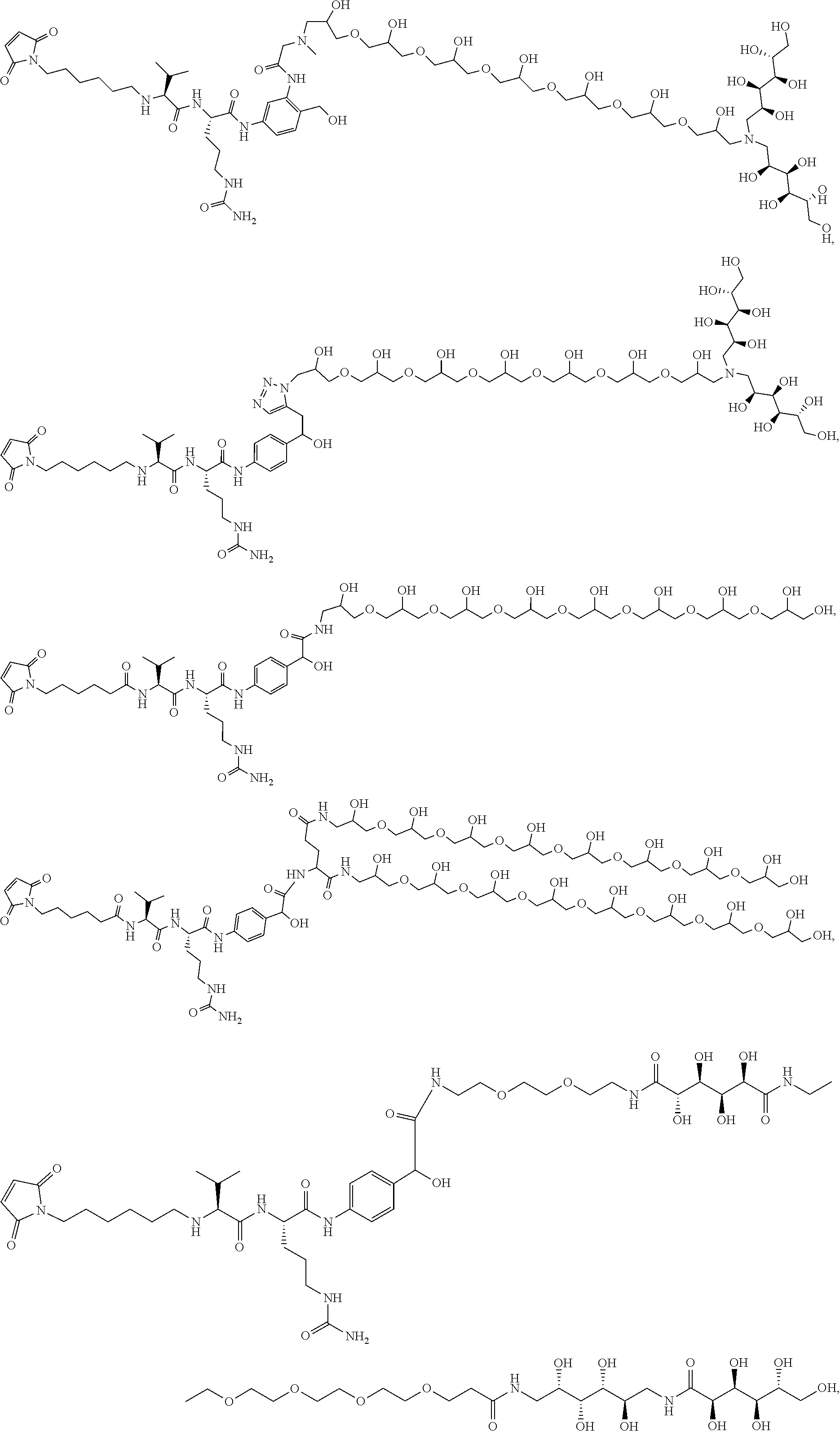 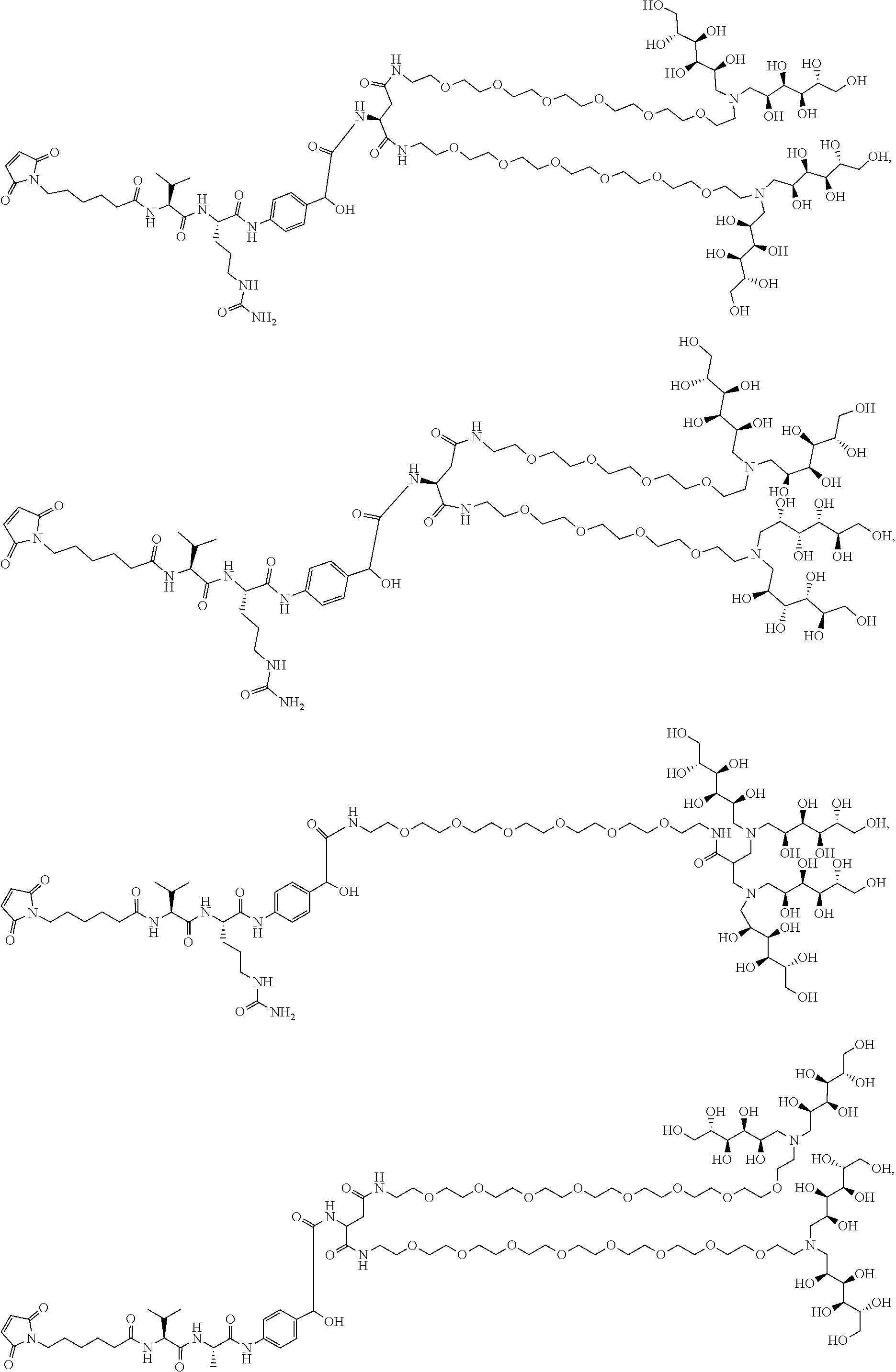 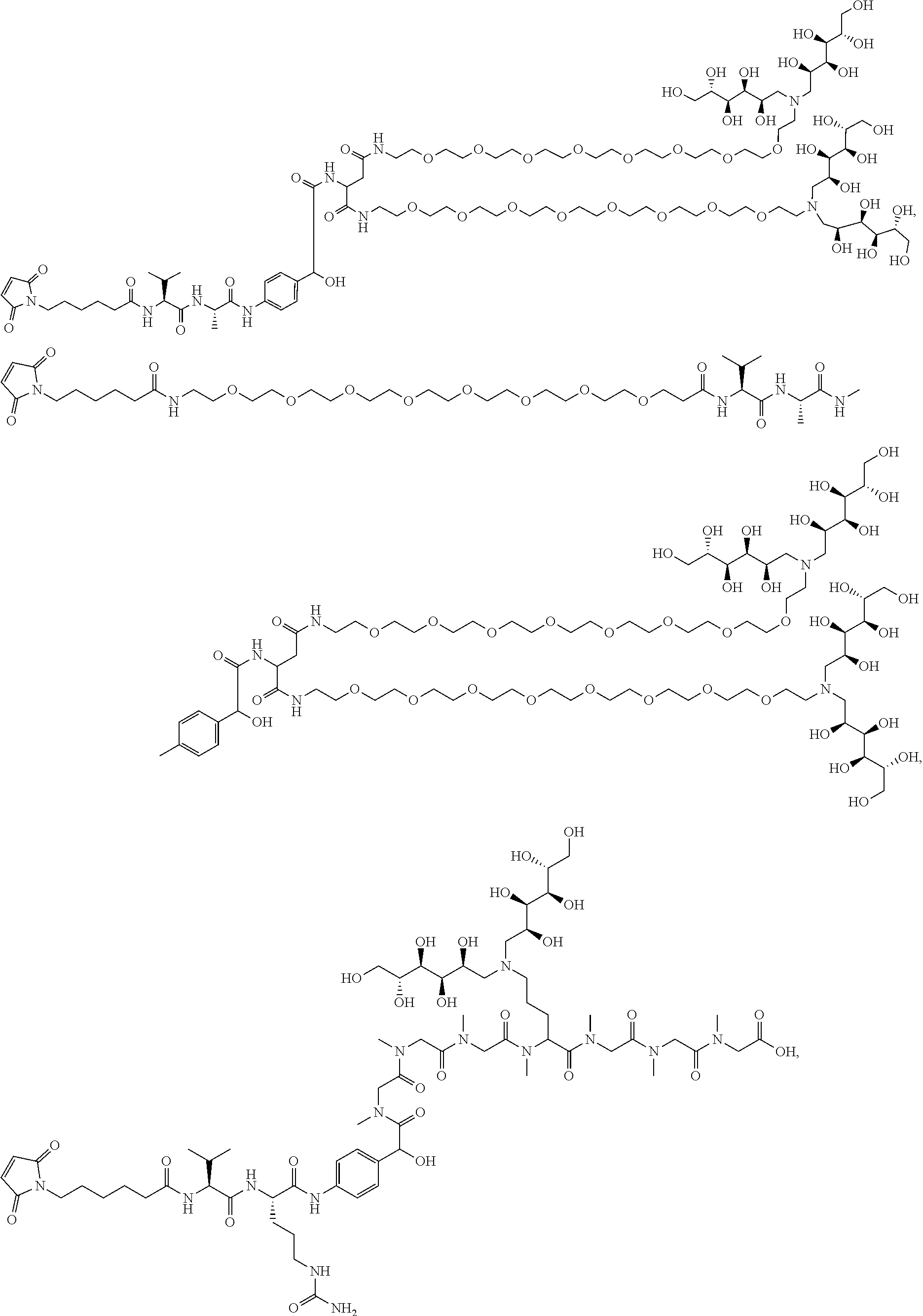 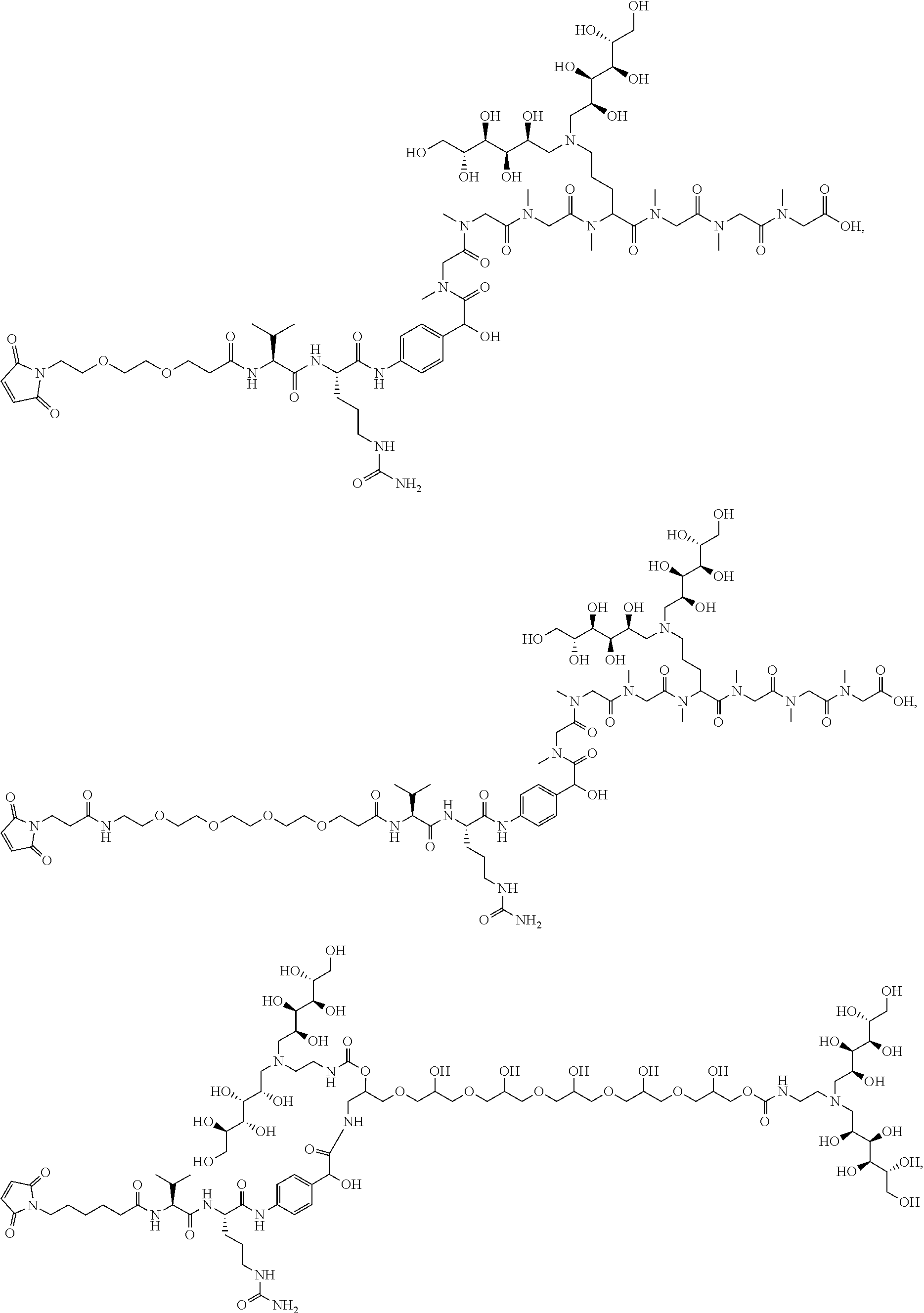 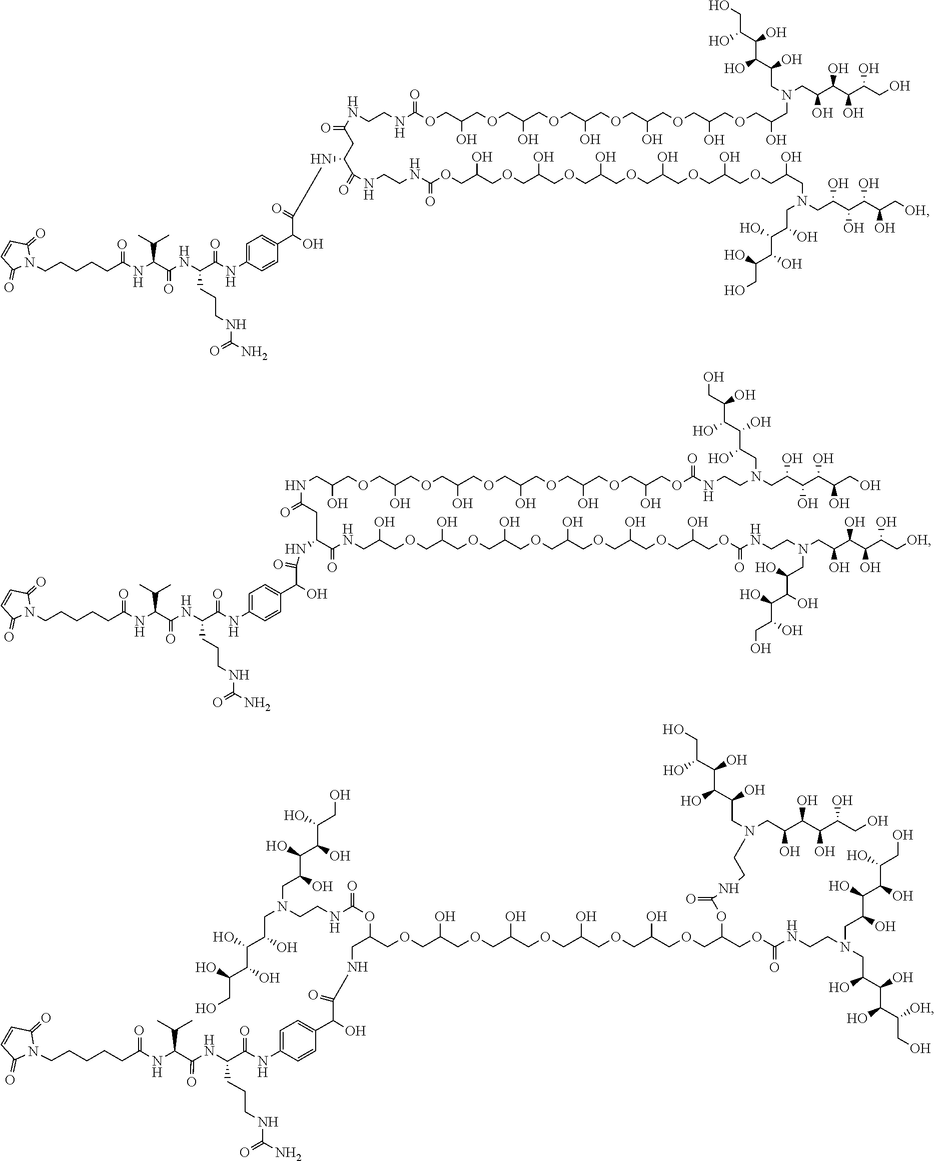 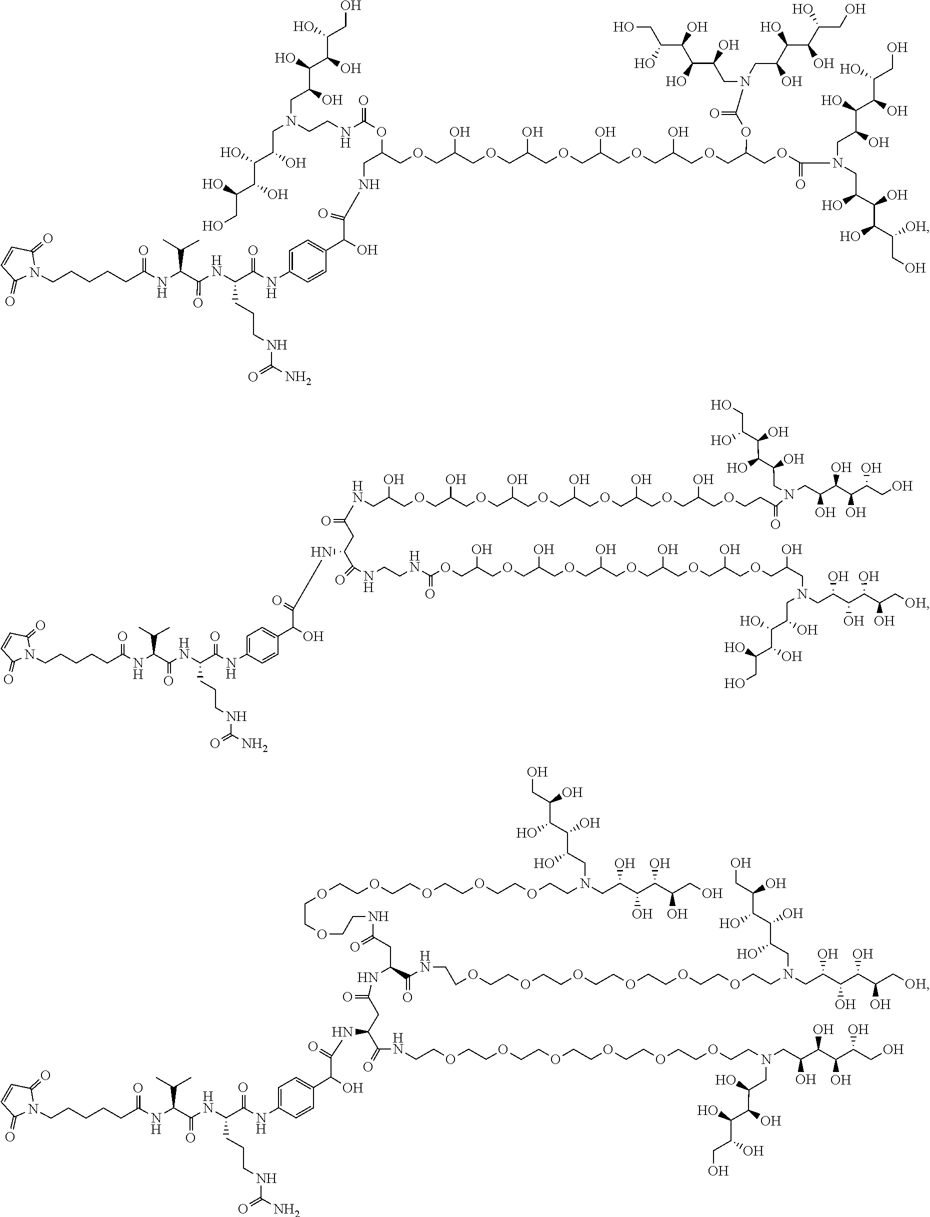 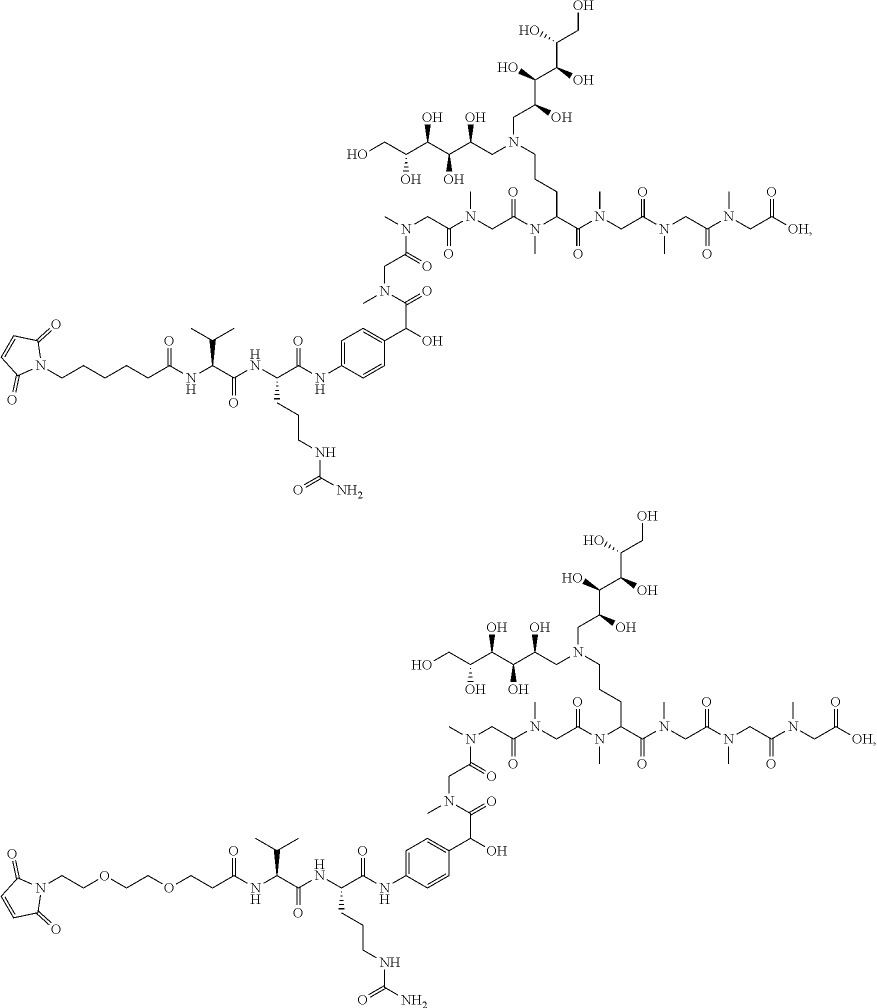 [SMILES: CC(C)[C@H](NC(=O)CCCCCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)O)cc1 ; CC(C)[C@H](NC(=O)CCOCCOCCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)O)cc1] 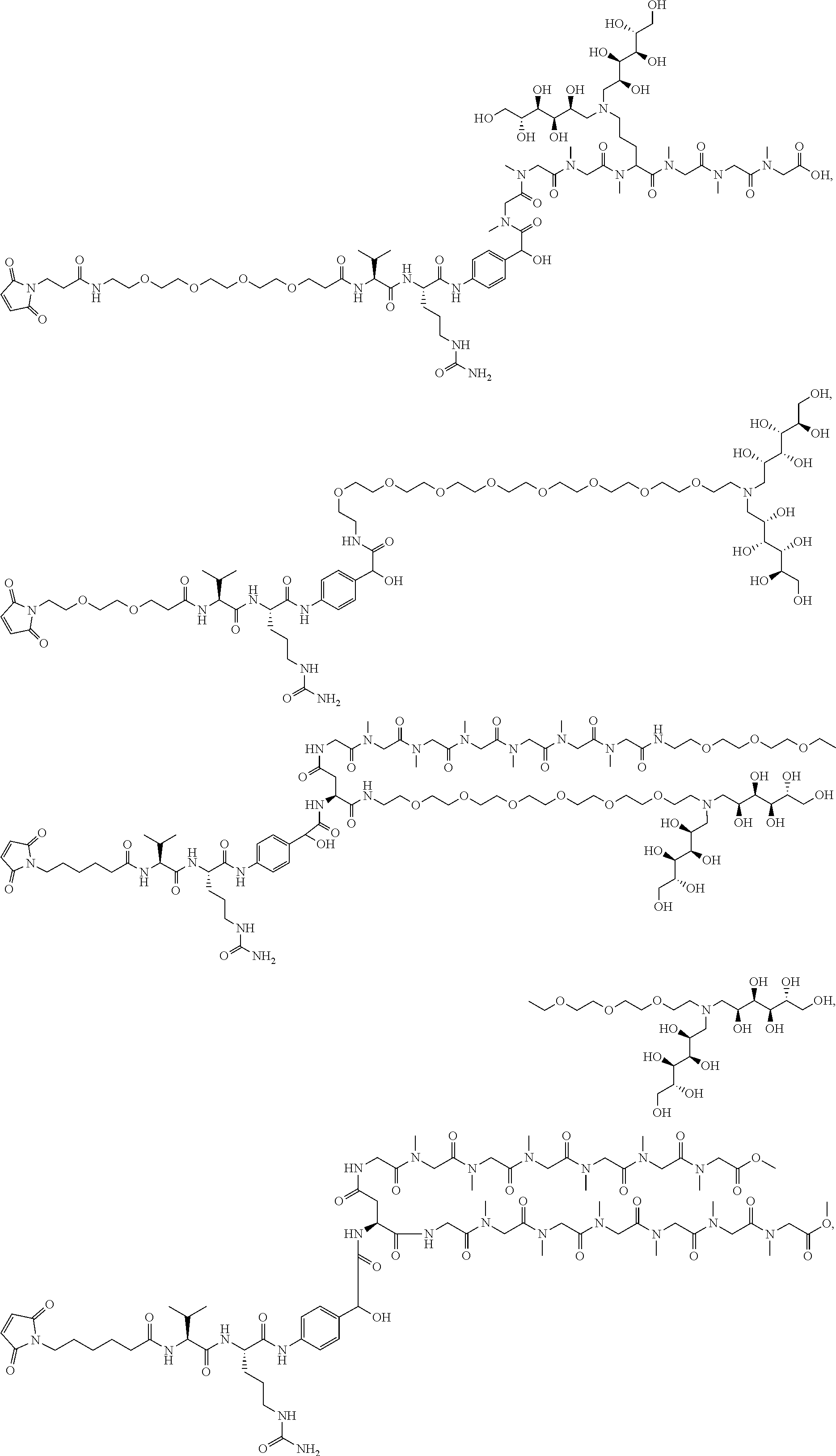 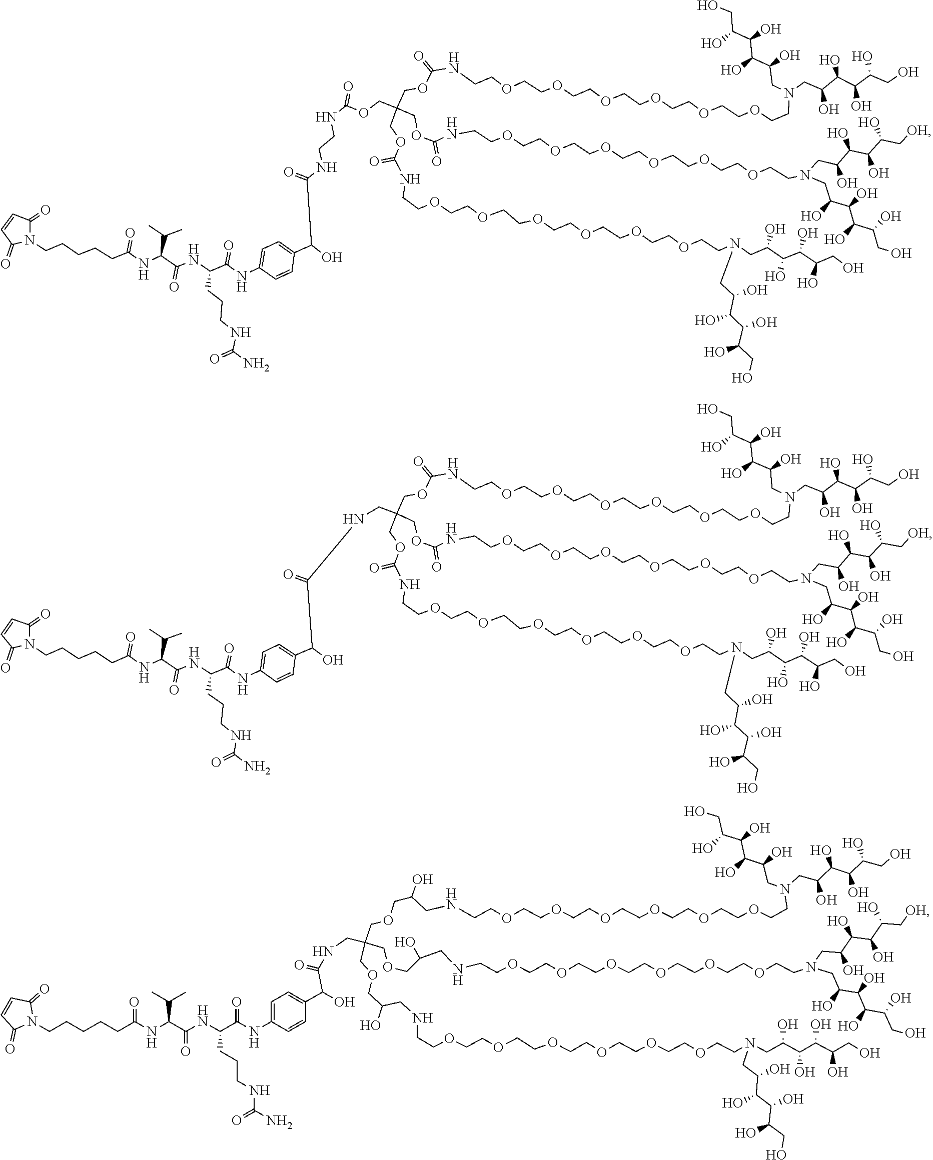 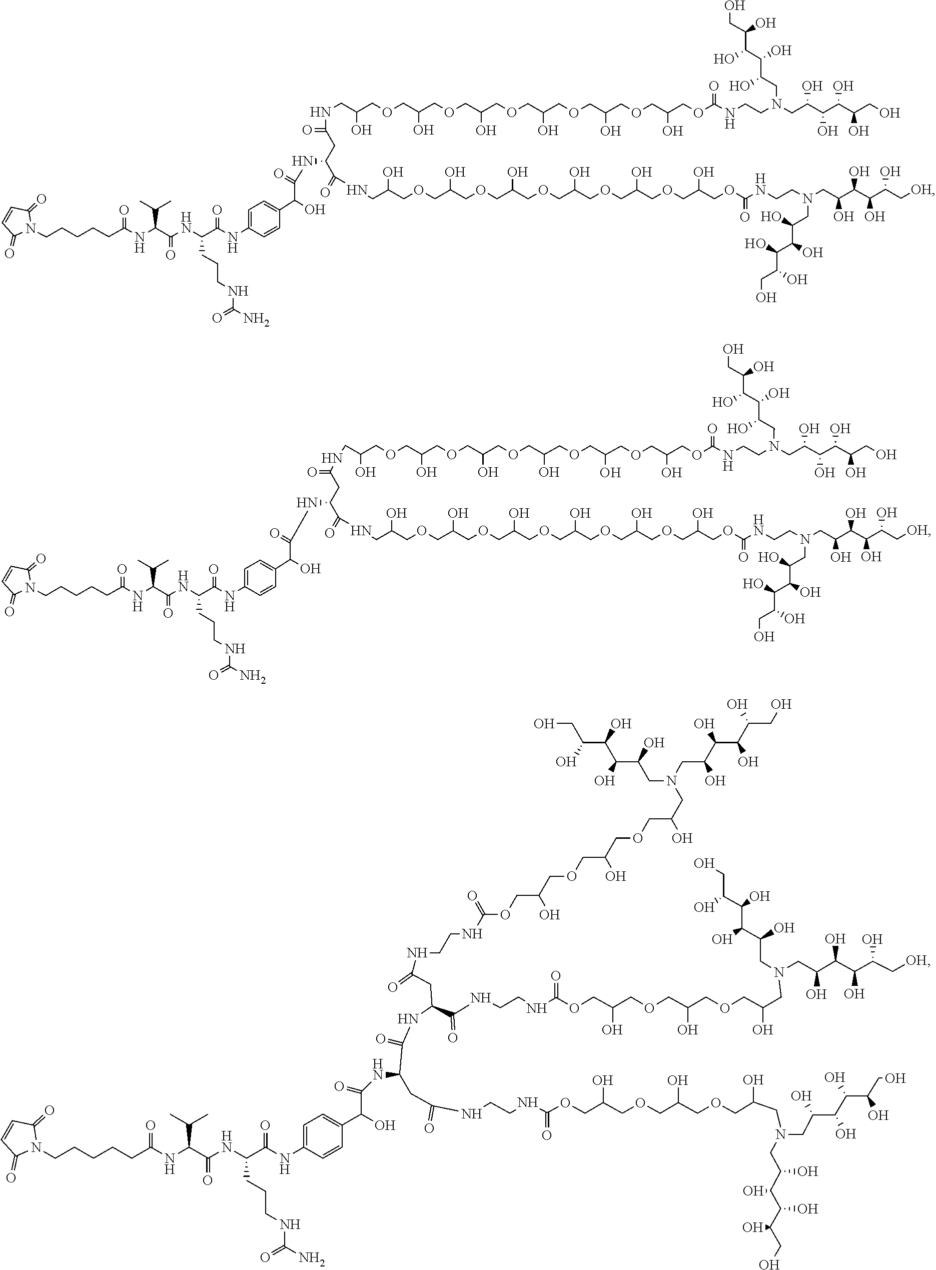 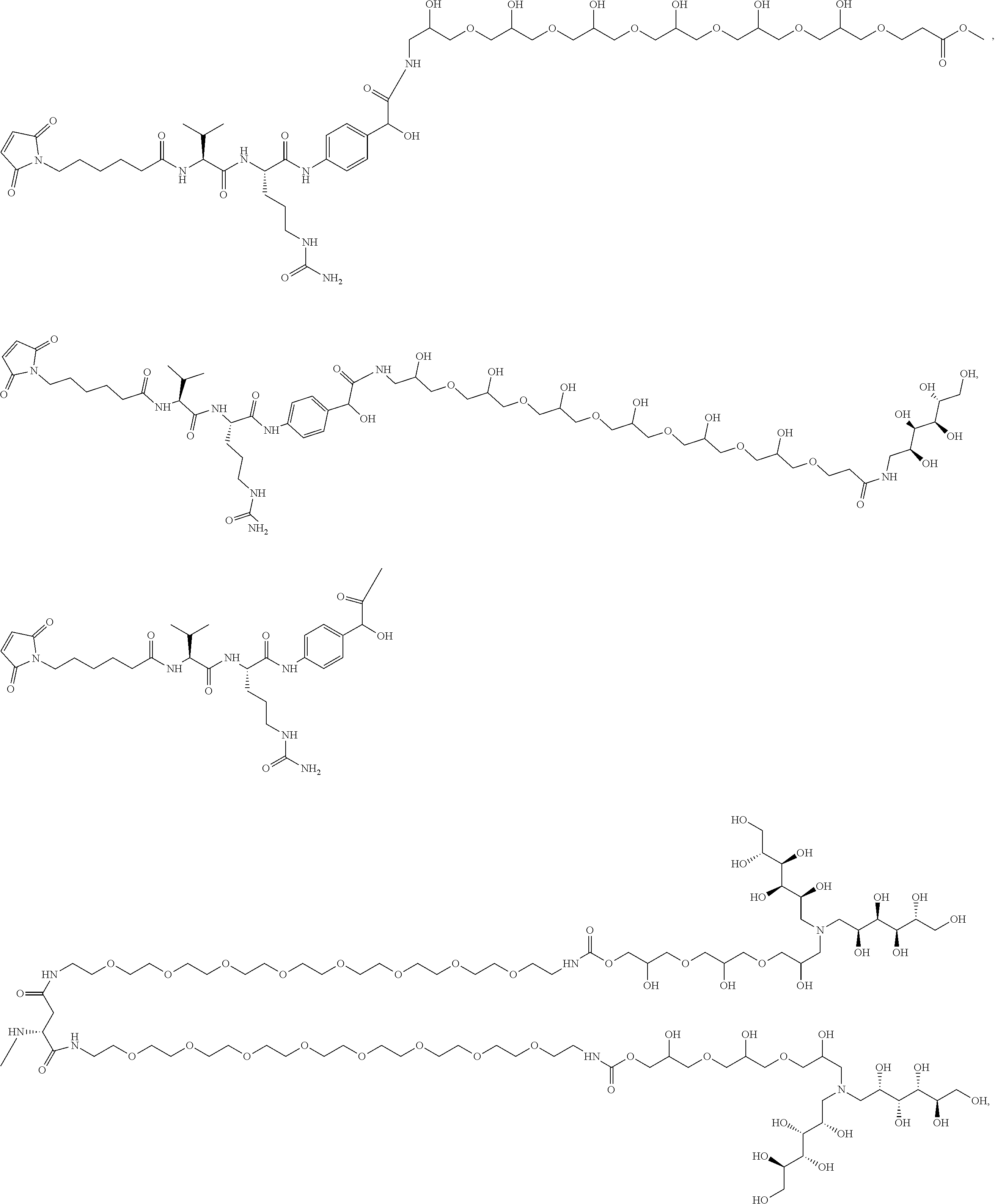 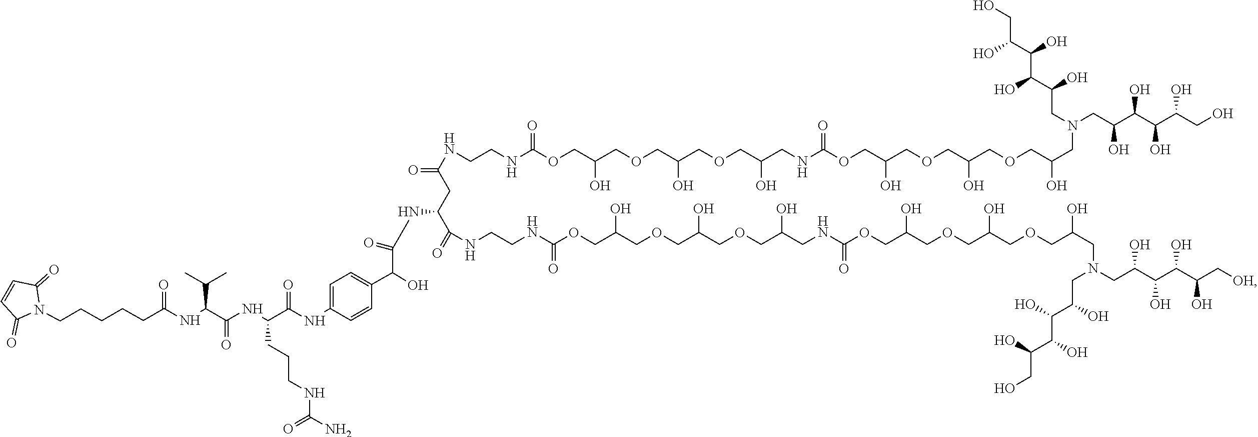 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCCNC(=O)OCC(O)COCC(O)COCC(O)CNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)CNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)cc1] 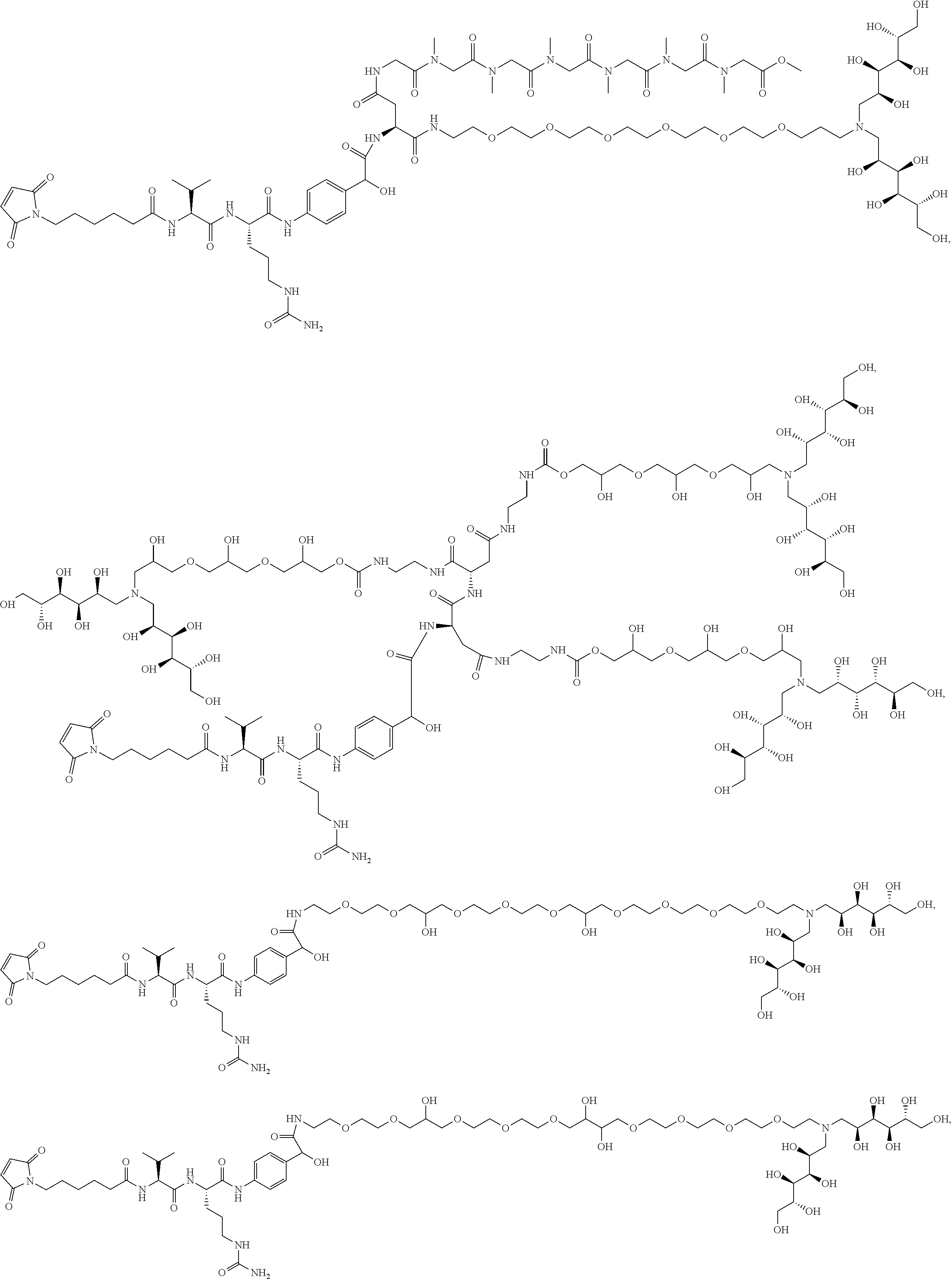 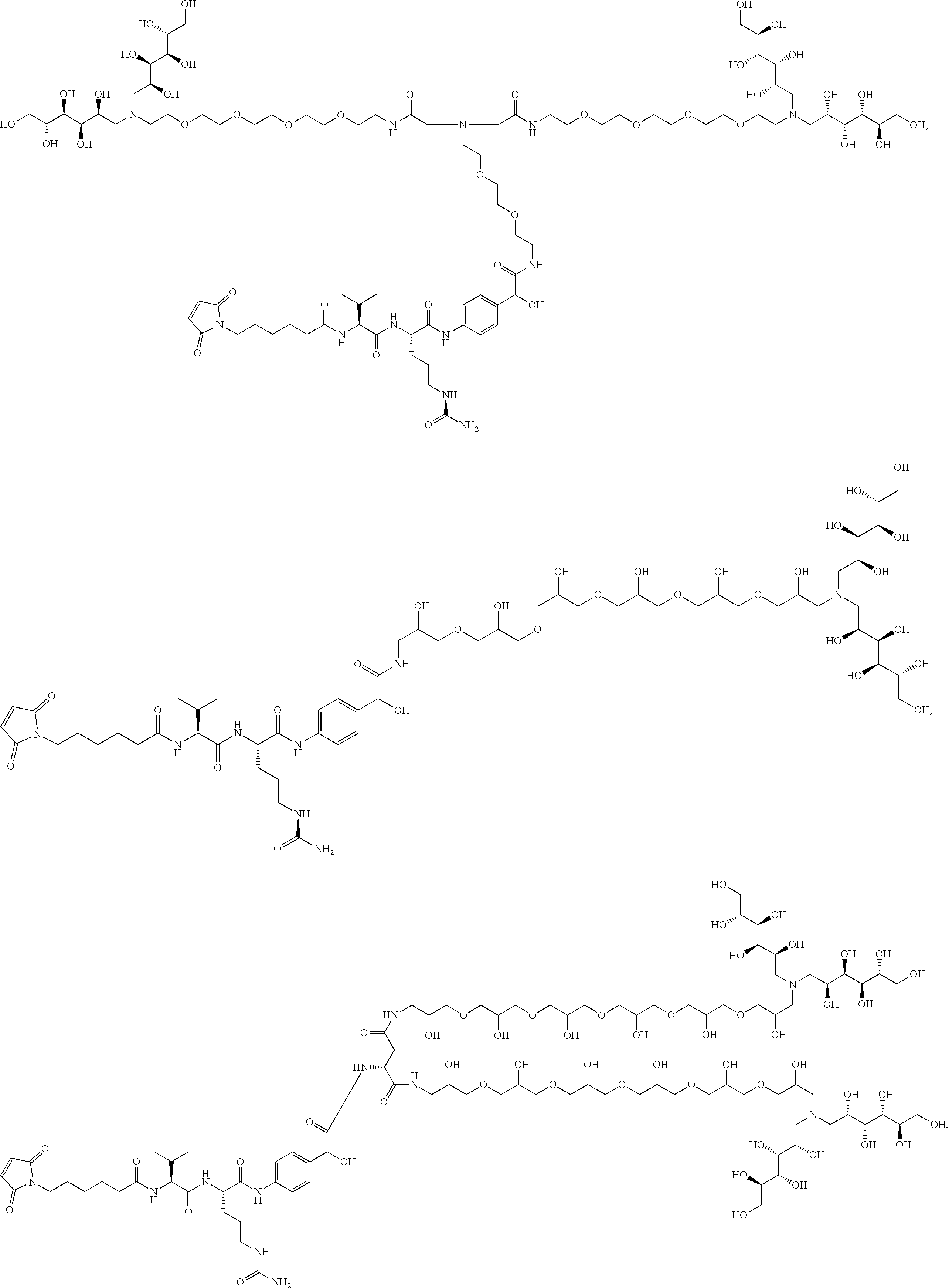 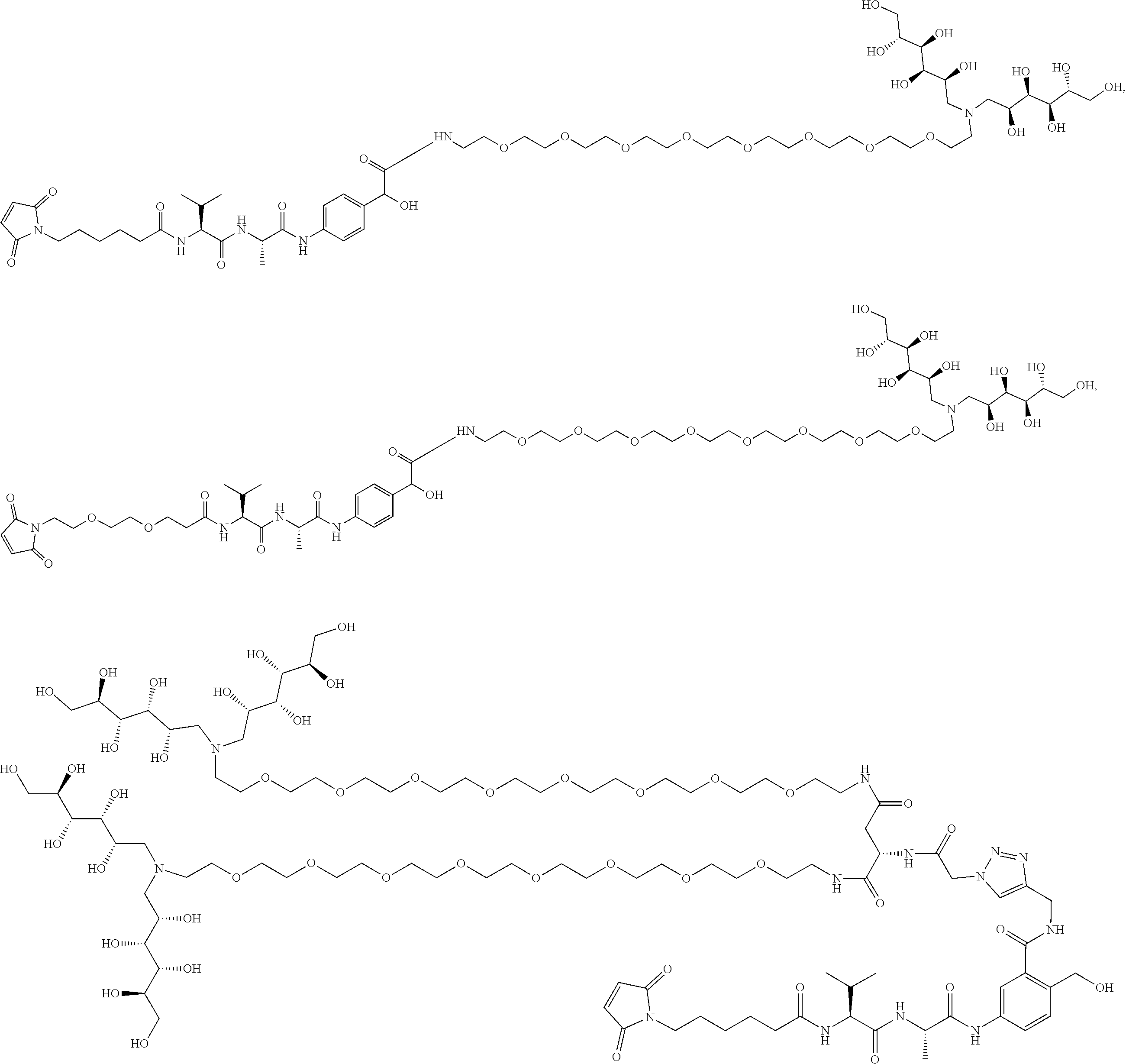
wherein the H on the benzylic OH is optionally replaced with a bond to the at least one Drug unit or to the linking group attached to the at least one Drug unit.

78. A Drug-Linker compound, comprising a Linker compound of any one of the claims 1-77 attached to the at least one Drug unit, or attached to the linking group attached to the at least one Drug unit, at the attachment site.

79. The Drug-Linker of claim 78, wherein the Drug unit is selected from a cytotoxic agent, an immune modulatory agent, a nucleic acid, a growth inhibitory agent, a PROTAC, a toxin, a radioactive isotope, and a chelating ligand.

80. The Drug-Linker of claim 79, wherein the Drug unit is a cytotoxic agent.

81. The Drug-Linker of claim 80, wherein the cytotoxic agent is selected from the group consisting of an auristatin, a maytansinoid, a camptothecin, a duocarmycin, and a calicheamicin.

82. The Drug-Linker of claim 81, wherein the cytotoxic agent is an auristatin.

83. The Drug-Linker of claim 82, wherein the cytotoxic agent is MMAE or MMAF.

84. The Drug-Linker of claim 81, wherein the cytotoxic agent is a camptothecin.

85. The Drug-Linker of claim 84, wherein the cytotoxic agent is exatecan, or SN-38, or DxD.

86. The Drug-Linker of claim 85, wherein the cytotoxic agent is RS-exatecan or SS-exatecan.

87. The Drug-Linker of claim 81, wherein the cytotoxic agent is a calicheamicin.

88. The Drug-Linker of claim 81, wherein the cytotoxic agent is a maytansinoid.

89. The Drug-Linker of claim 88, wherein the maytansinoid is maytansine, maytansinol, or ansamatocin-2.

90. The Drug-Linker of claim 79, wherein the Drug unit is an immune modulatory agent.

91. The Drug-Linker of claim 90, wherein the immune modulatory agent is selected from a TRL7 agonist, a TLR8 agonist, a STING agonist, or a RIG-1 agonist.

92. The Drug-Linker of claim 91, wherein the immune modulatory agent is an TLR7 agonist.

93. The Drug-Linker of claim 92, wherein the TLR7 agonist is an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, a benzonaphthyridine, a guanosine analog, an adenosine analog, a thymidine homopolymer, ssRNA, CpG-A, PolyG10, or PolyG3.

94. The Drug-Linker of claim 91, wherein the immune modulatory agent is a TLR8 agonist.

95. The Drug-Linker of claim 94, wherein the TLR8 agonist is selected from an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, or a ssRNA.

96. The Drug-Linker of claim 91, wherein the immune modulatory agent is a STING agonist.

97. The Drug-Linker of claim 91, wherein the immune modulatory agent is a RIG-1 agonist.

98. The Drug-Linker of claim 97, wherein the RIG-1 agonist is selected from KIN1148, SB-9200, KIN700, KIN600, KIN500, KIN100, KIN101, KIN400, and KIN2000.

99. The Drug-Linker of claim 79, wherein the Drug unit is a chelating ligand.

100. The Drug-Linker of claim 99, wherein the chelating ligand is selected from platinum (Pt), ruthenium (Ru), rhodium (Rh), gold (Au), silver (Ag), copper (Cu), molybdenum (Mo), titanium (Ti), or iridium (Ir); a radioisotope such as yittrium-88, yittrium-90, technetium-99, copper-67, rhenium-188, rhenium-186, galium-66, galium-67, indium-111, indium-114, indium-115, lutetium-177, strontium-89, sararium-153, and lead-212.

101. The Drug-Linker of claim 78, having one of the following structures:

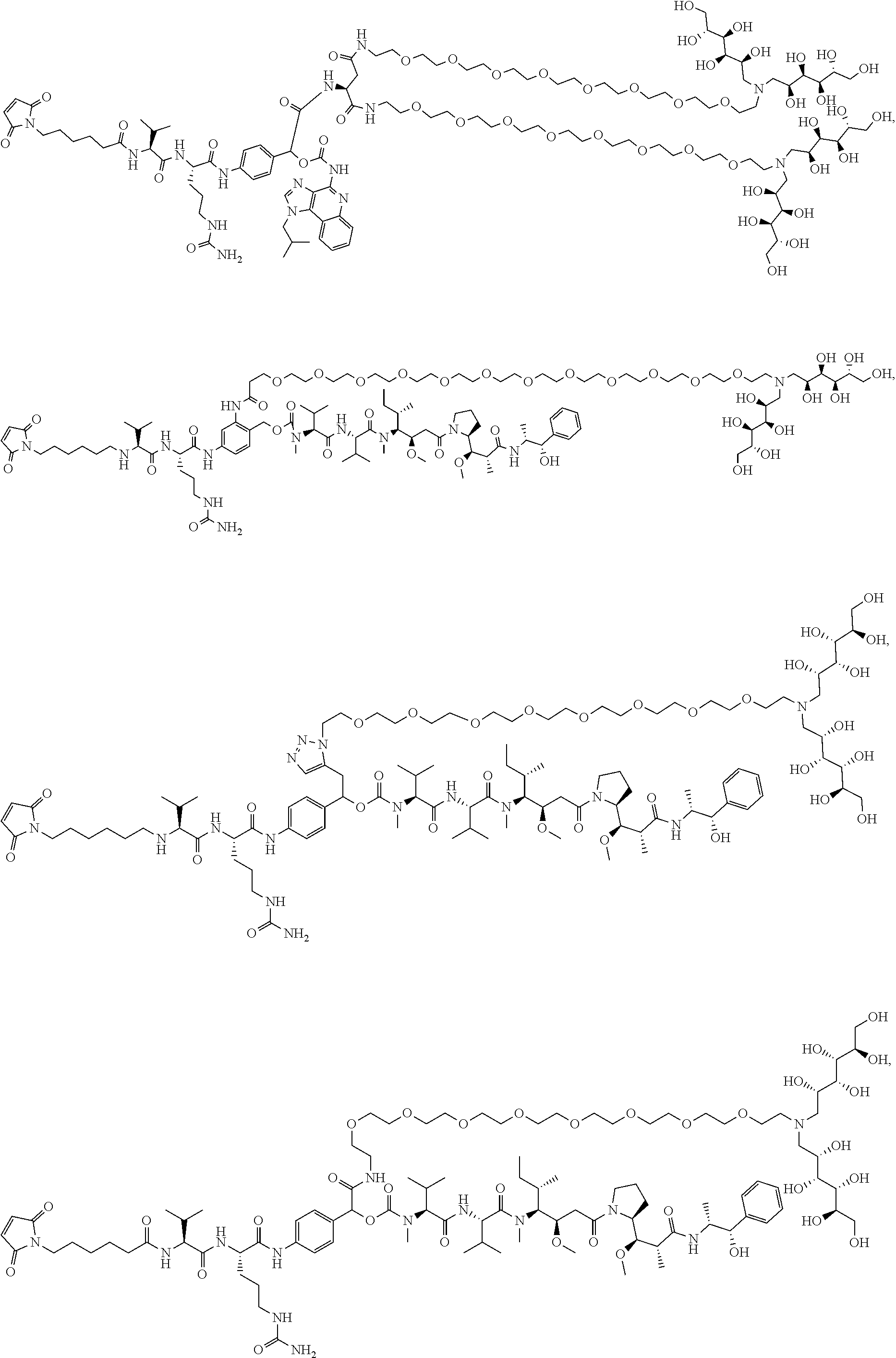

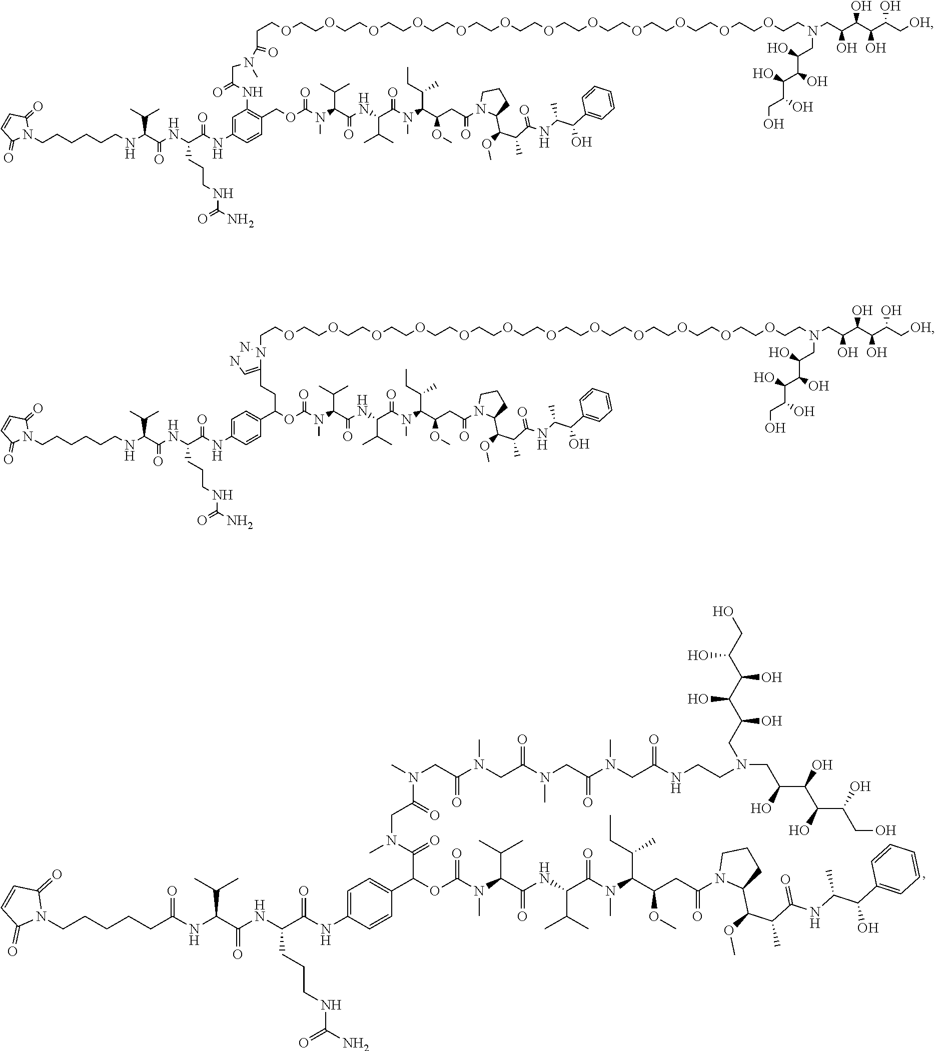

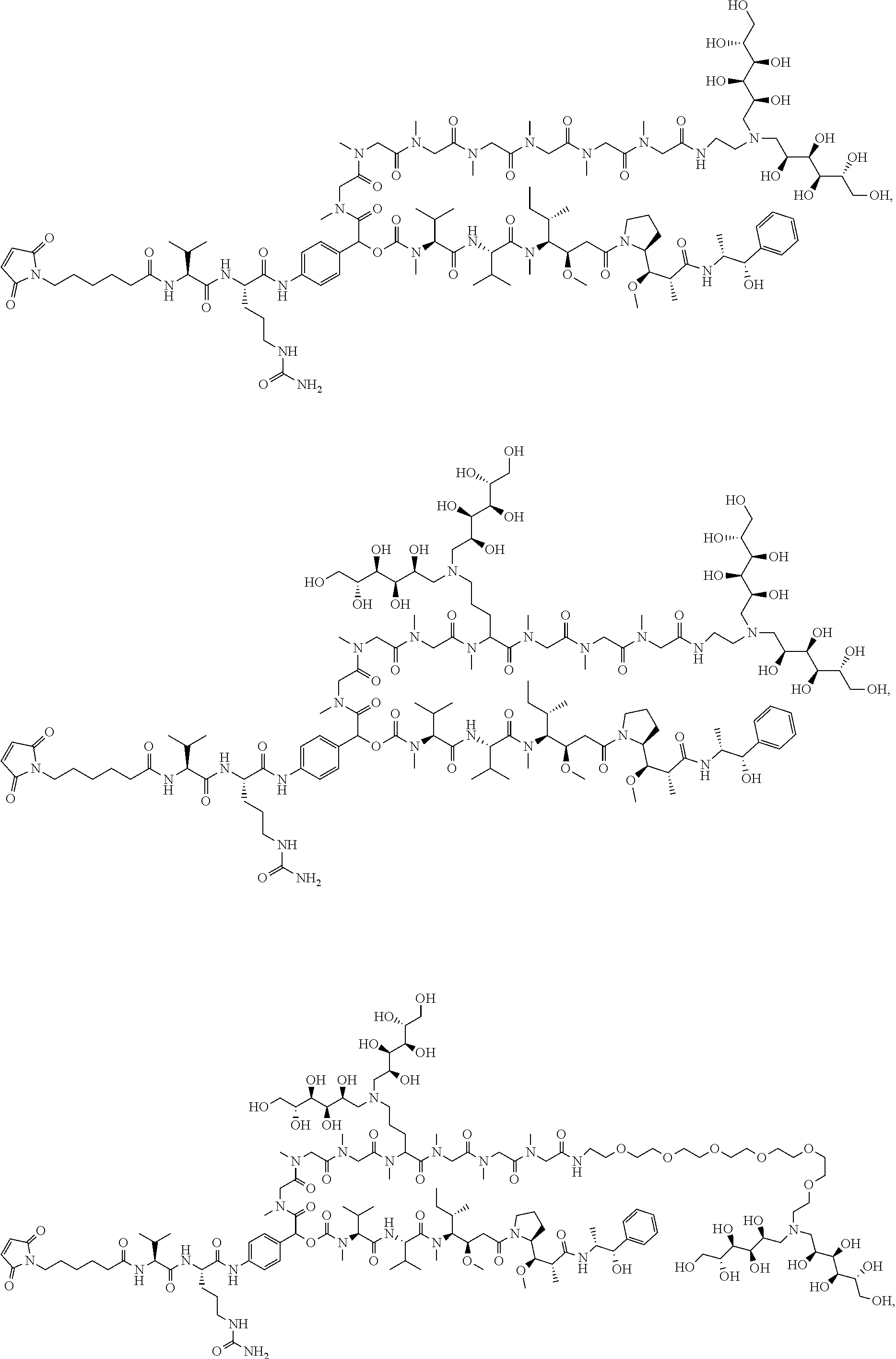

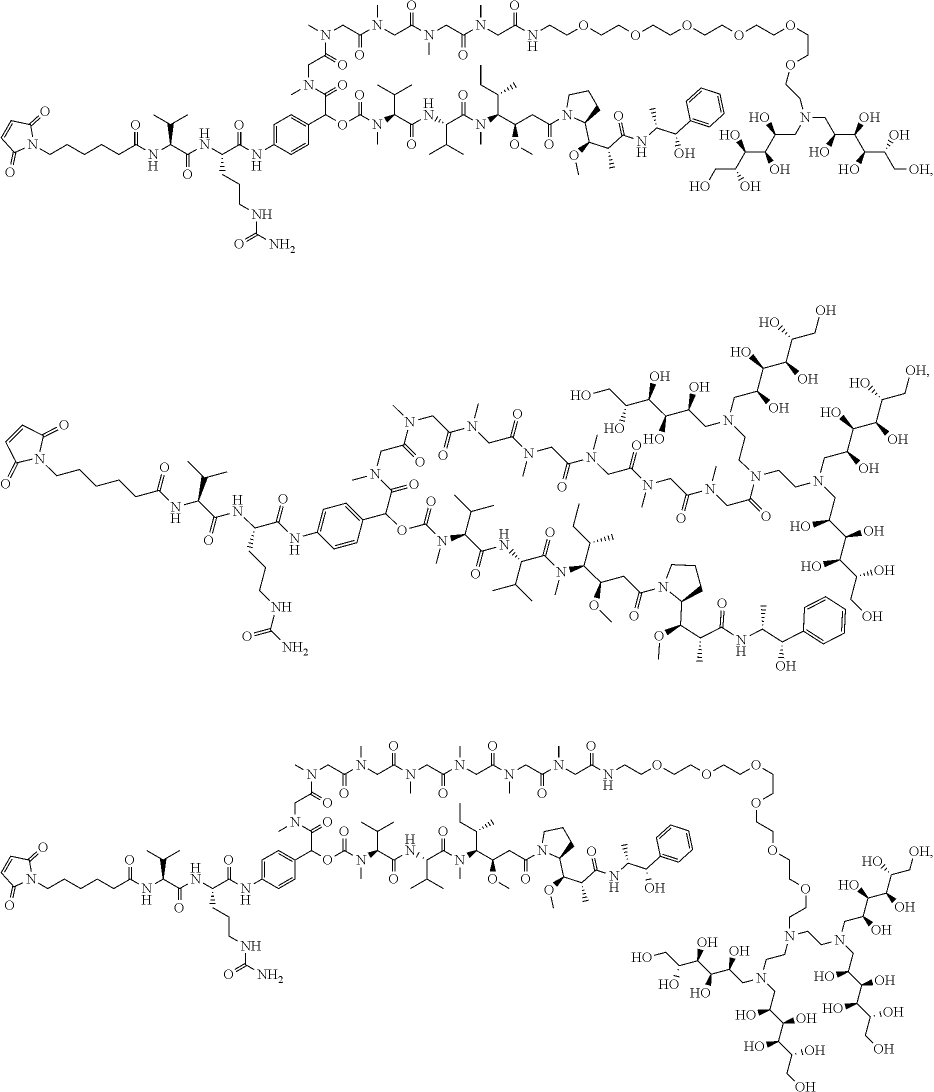

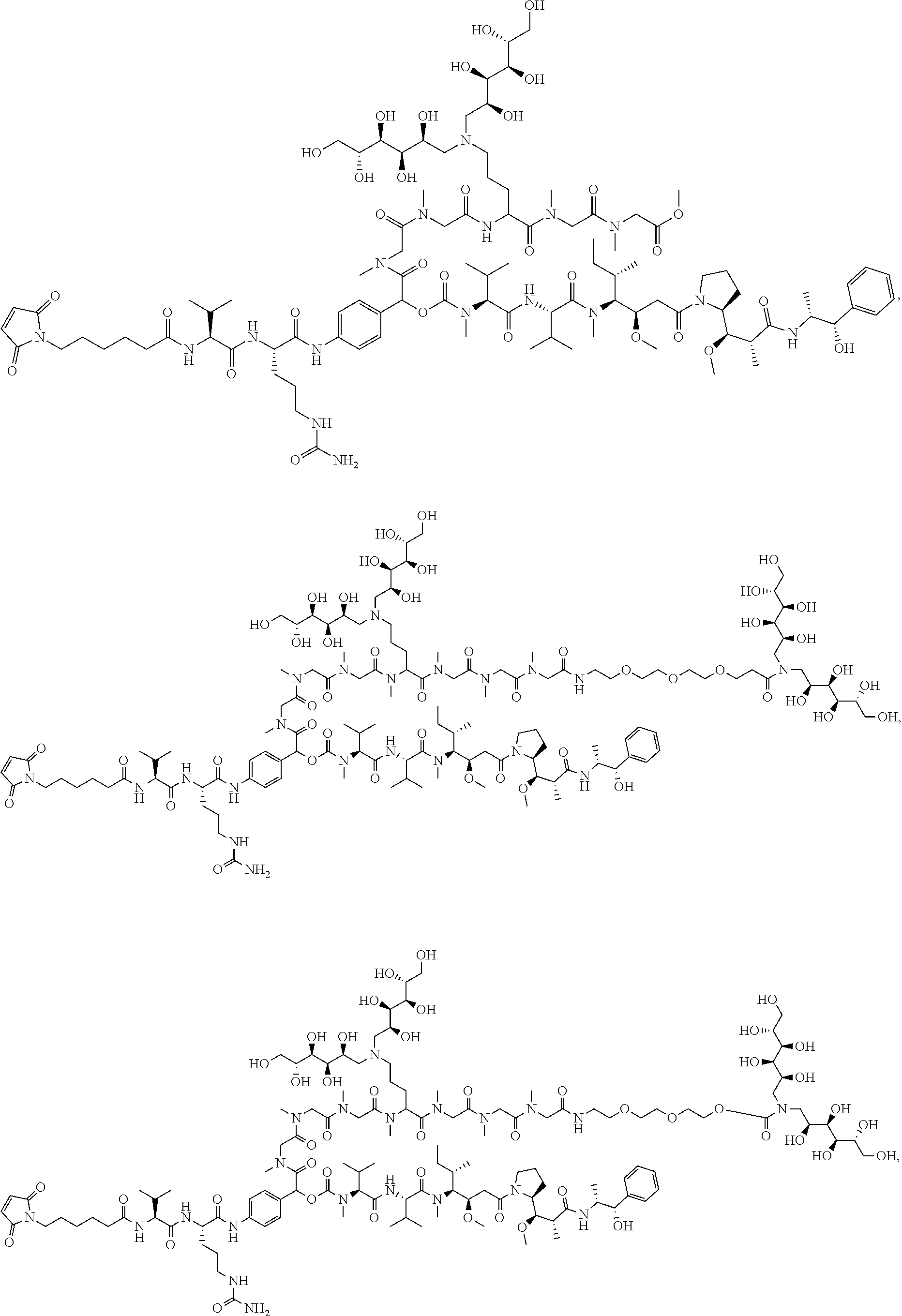

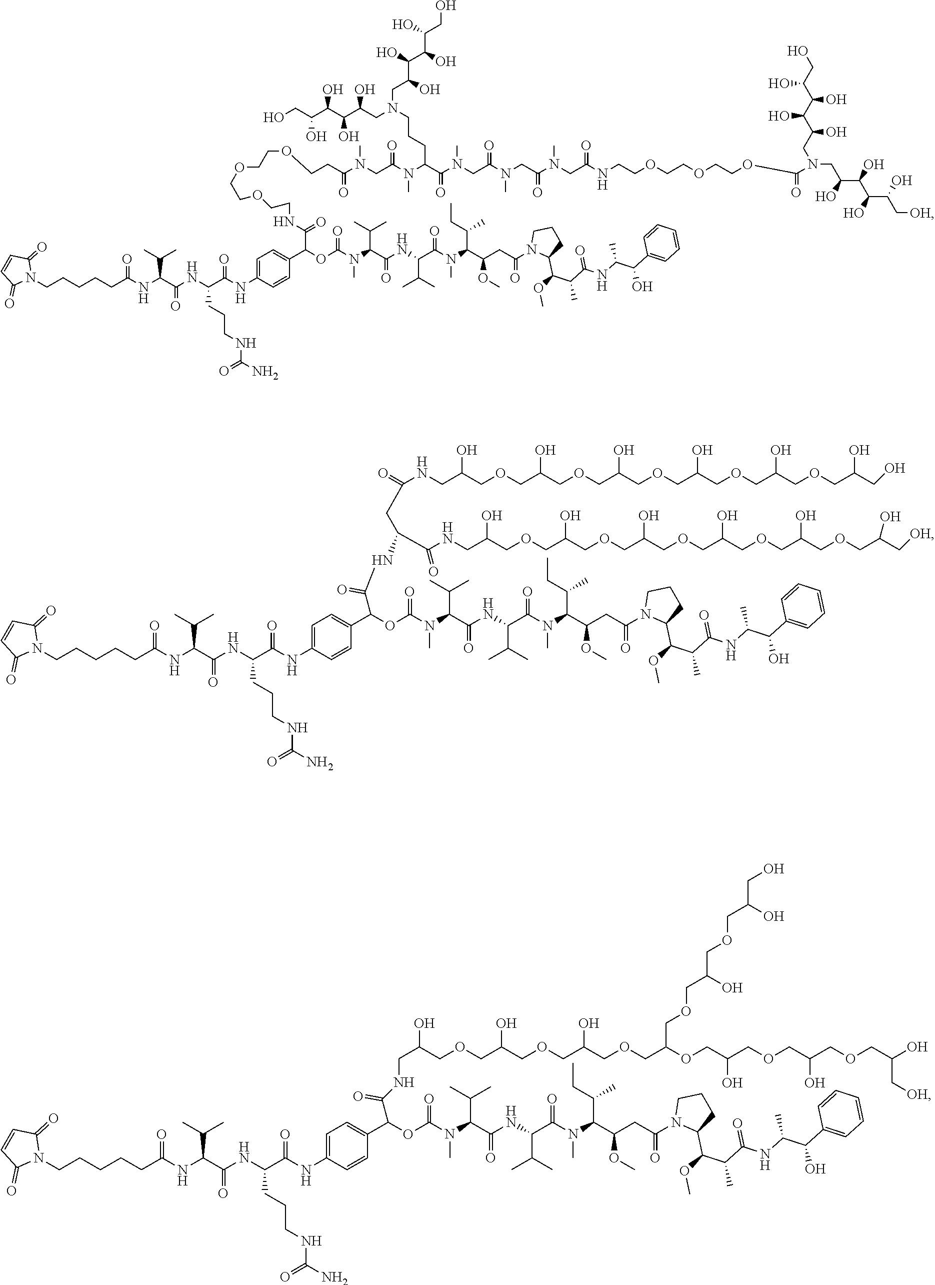

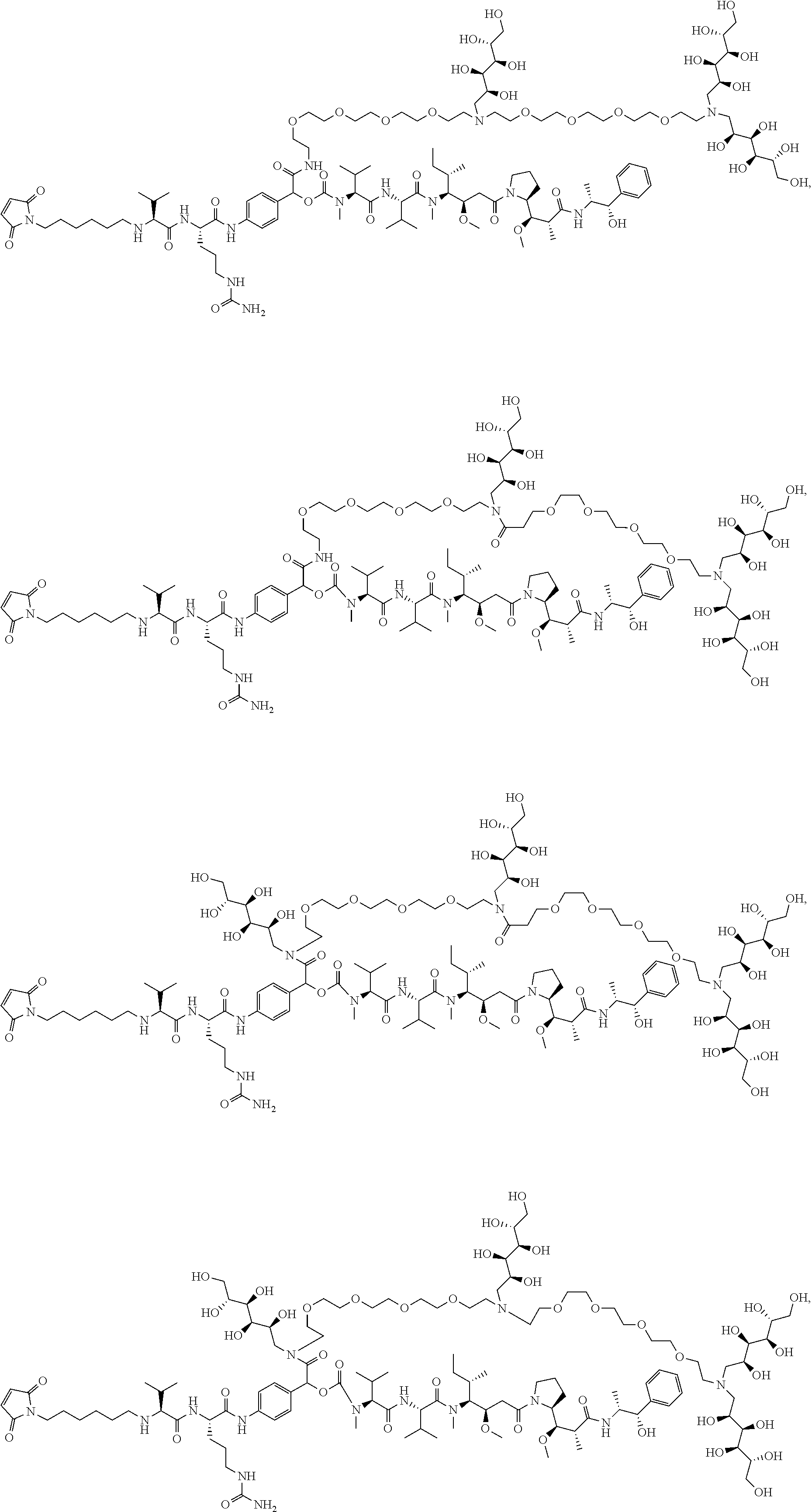

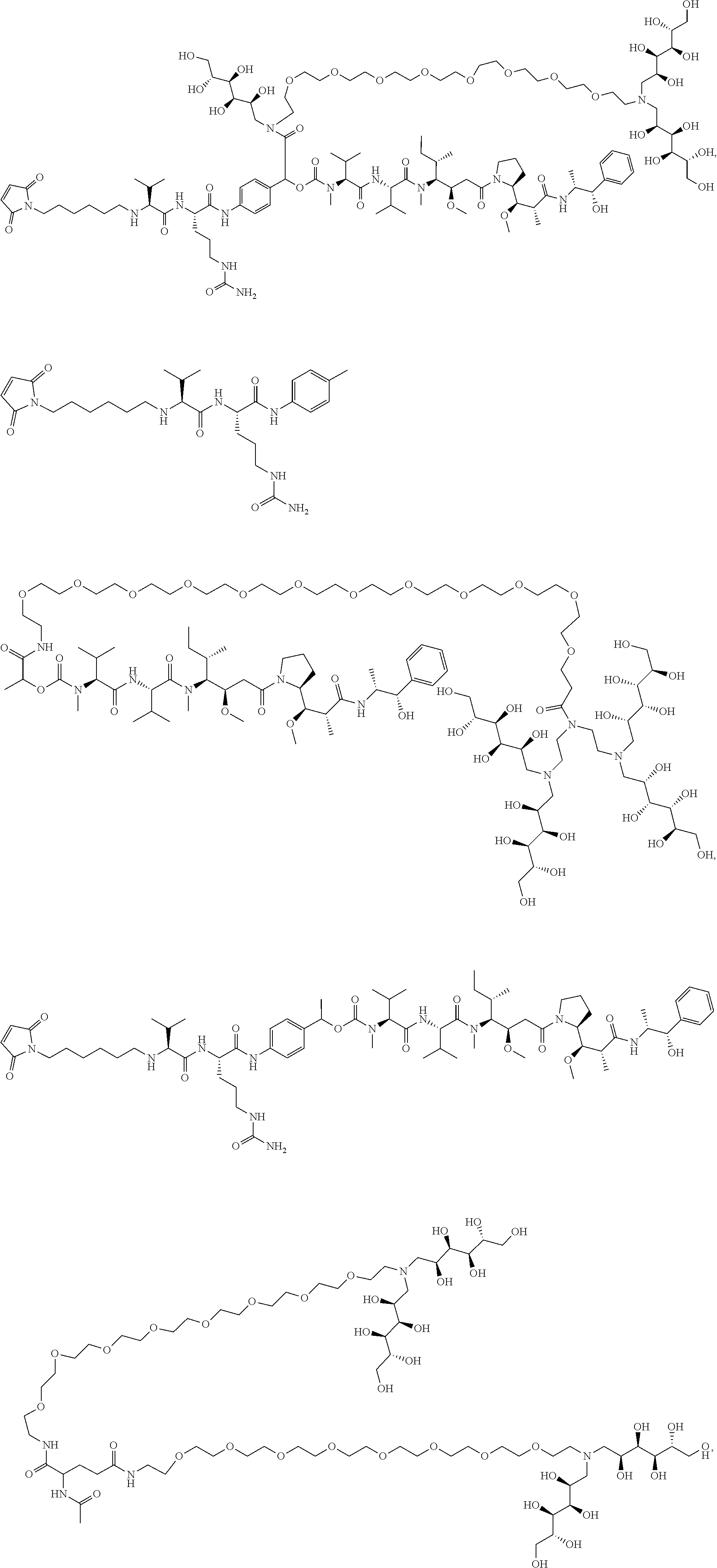

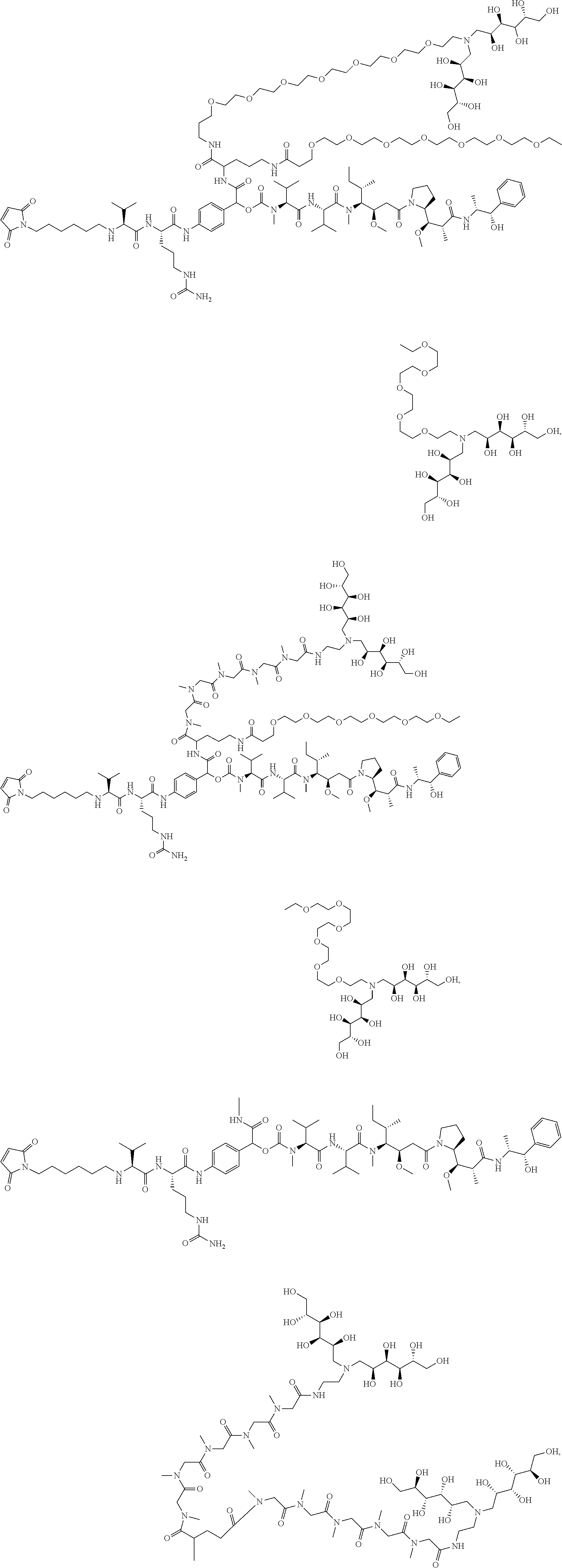

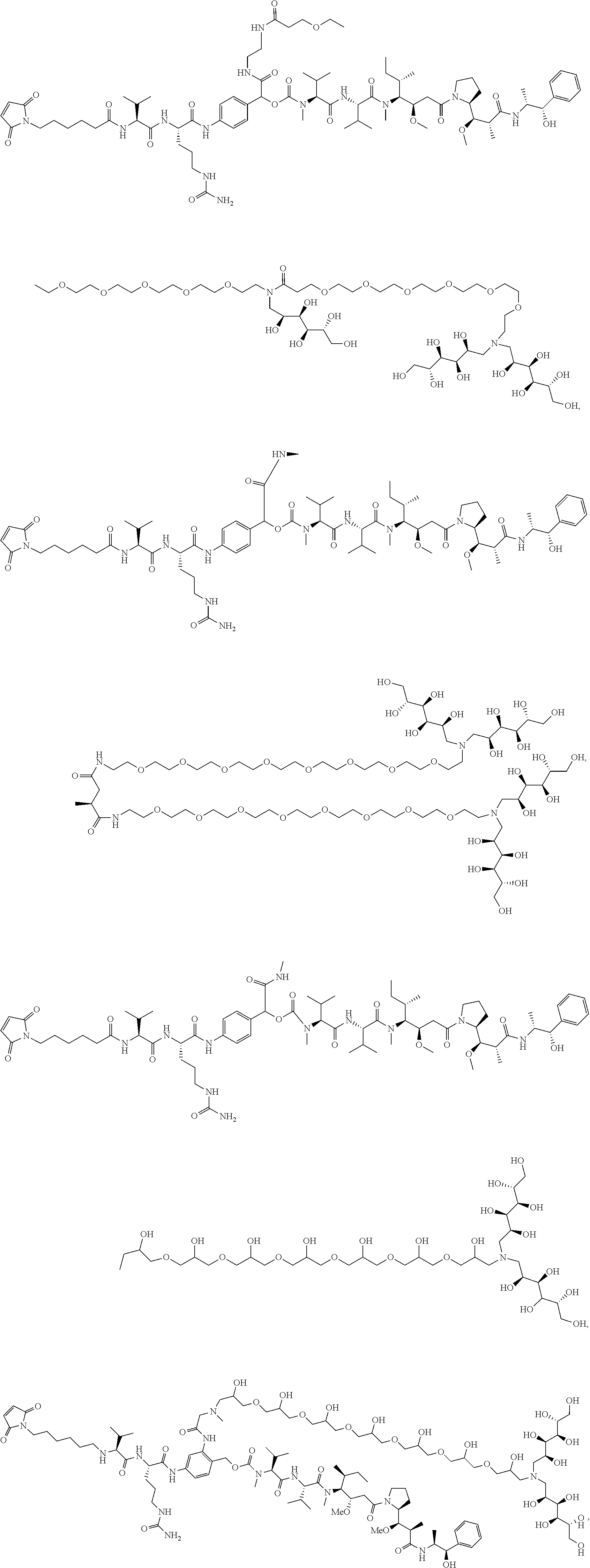

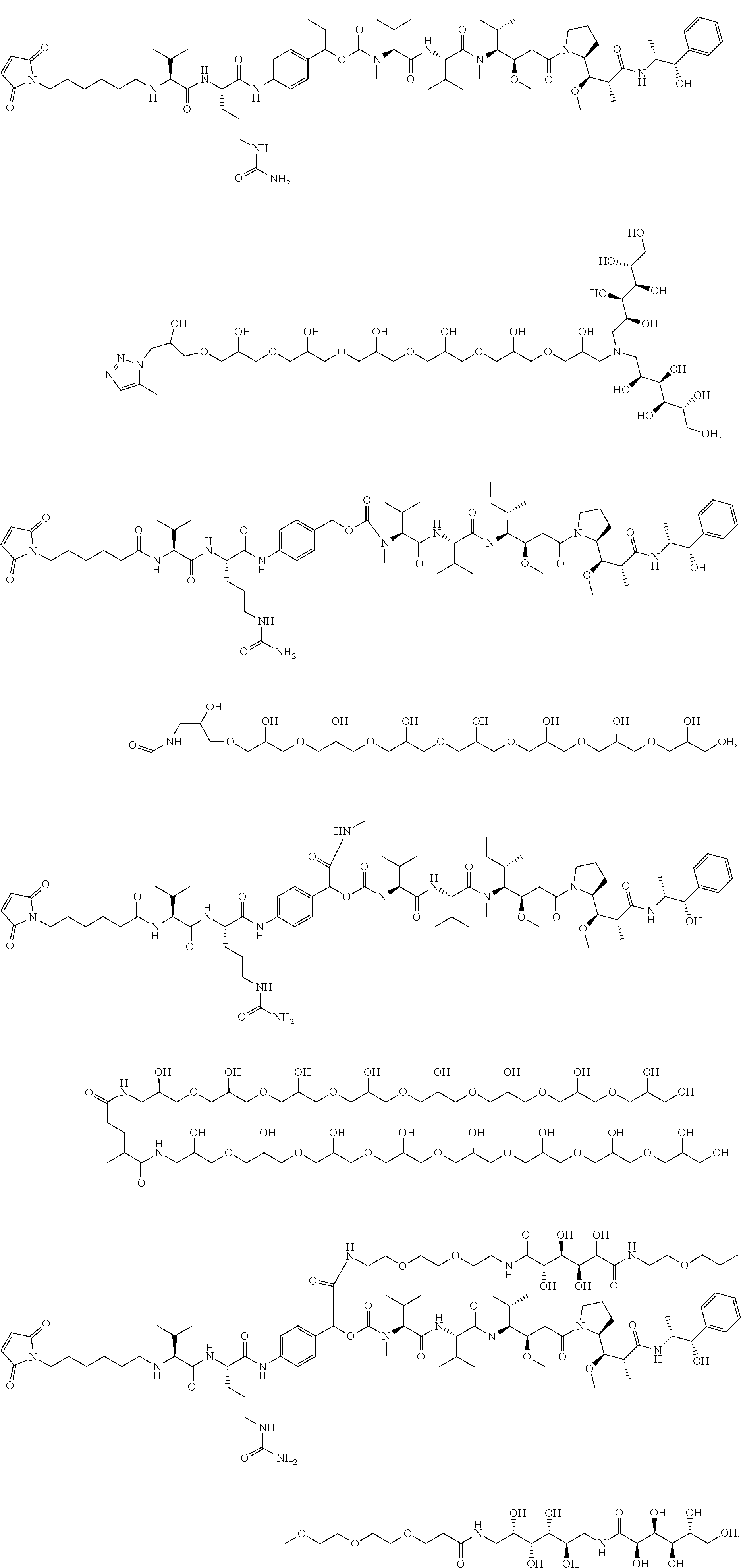

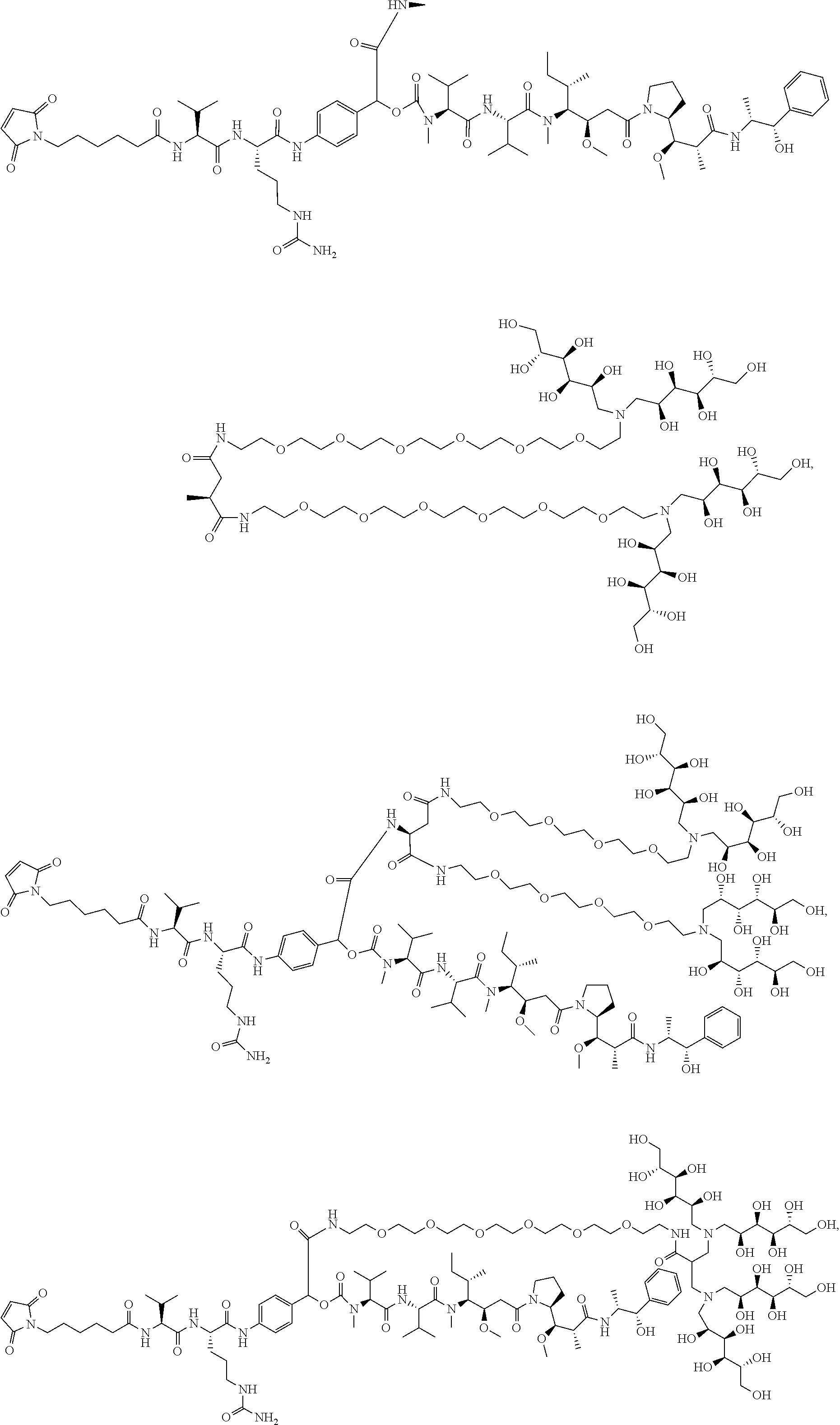

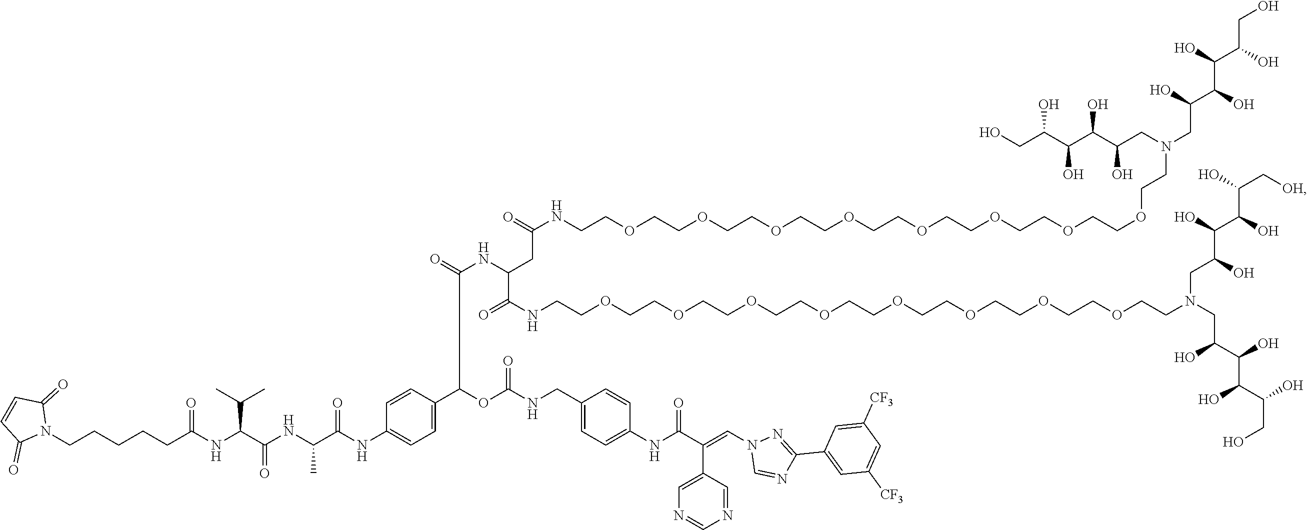 [SMILES: CC(C)[C@H](NC(=O)CCCCCN1C(=O)C=CC1=O)C(=O)N[C@@H](C)C(=O)Nc1ccc(C(OC(=O)NCc2ccc(NC(=O)/C(=C/n3cnc(-c4cc(C(F)(F)F)cc(C(F)(F)F)c4)n3)c3cncnc3)cc2)C(=O)NC(CC(=O)NCCOCCOCCOCCOCCOCCOCCOCCOCCN(C[C@@H](O)[C@H](O)[C@@H](O)[C@@H](O)CO)C[C@@H](O)[C@H](O)[C@@H](O)[C@@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

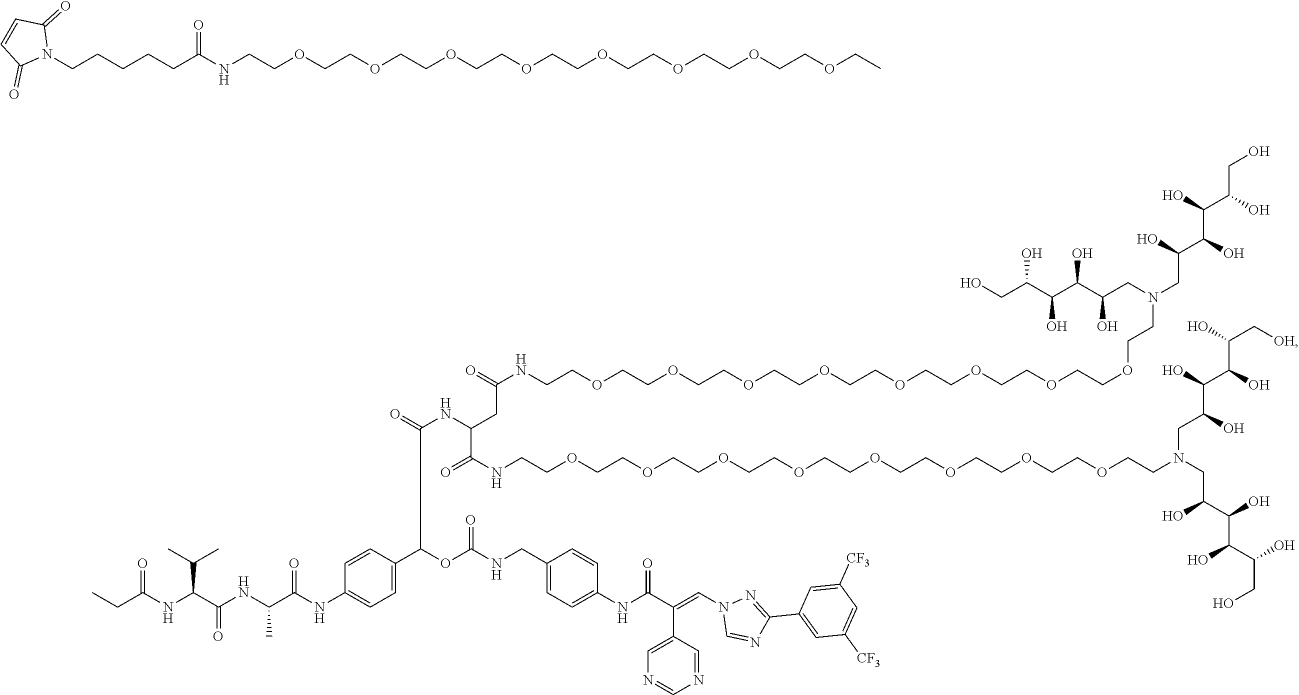

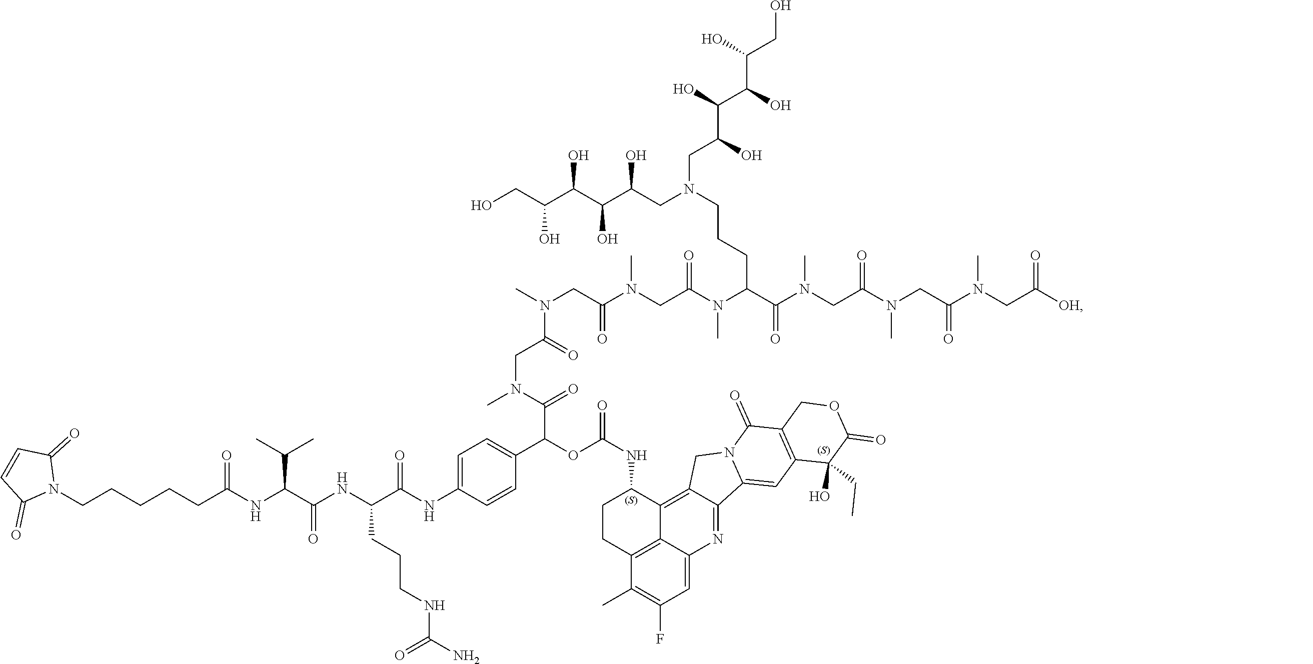

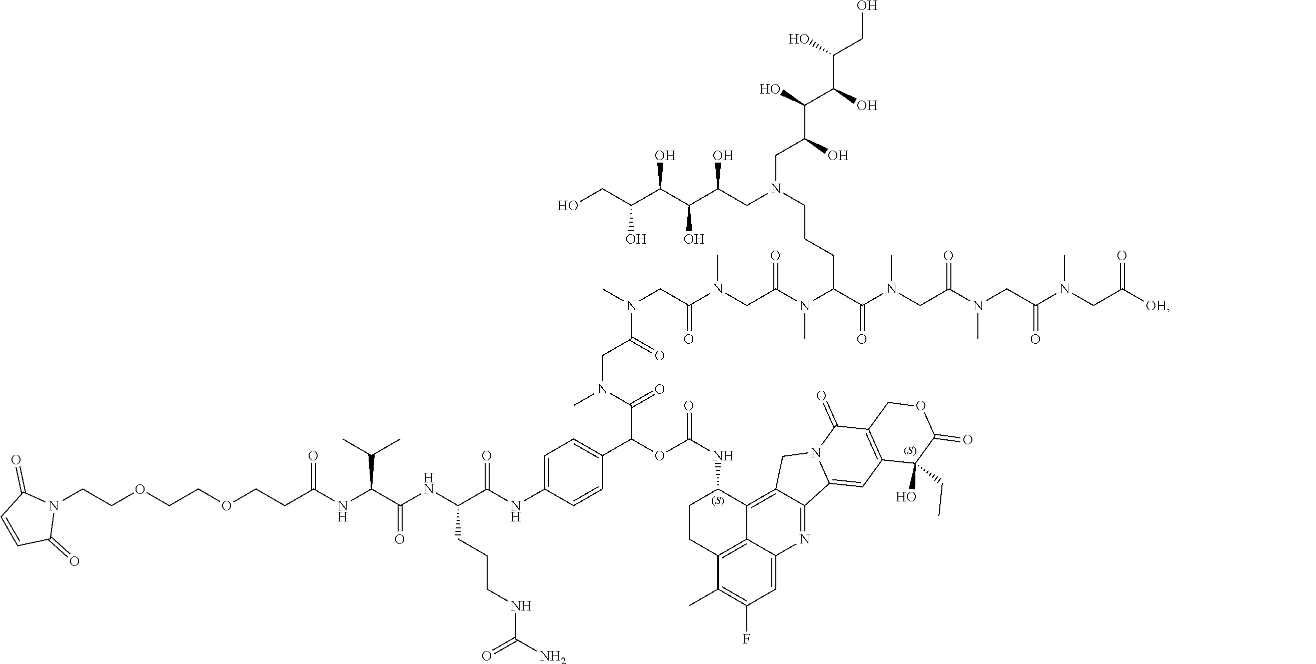

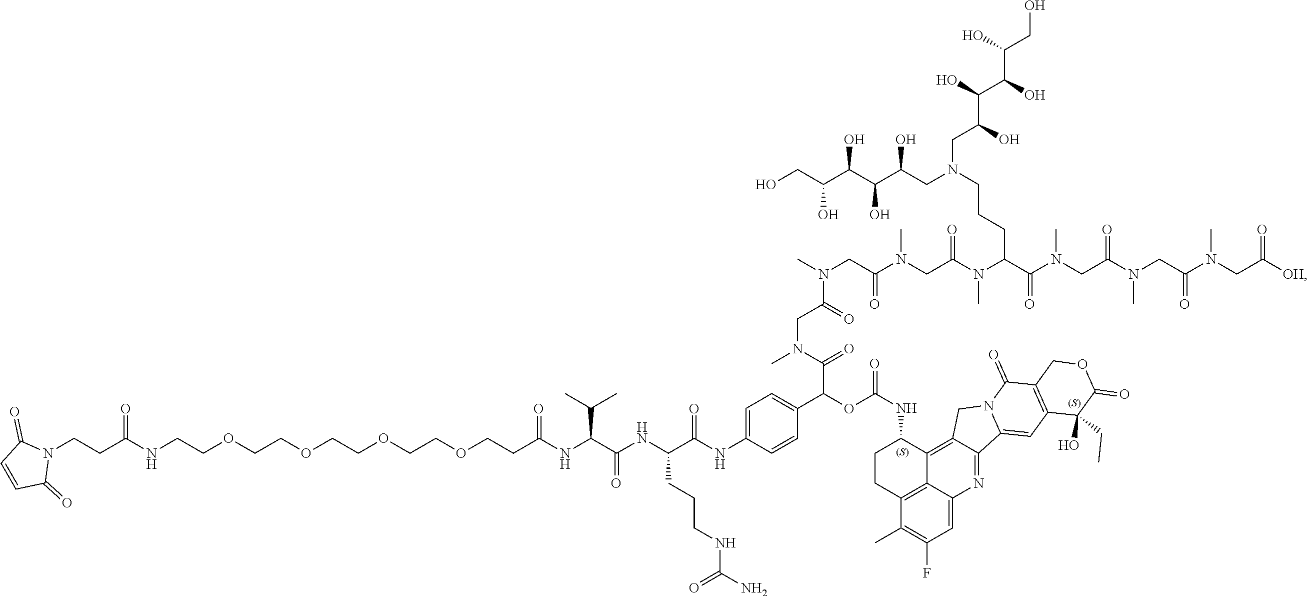

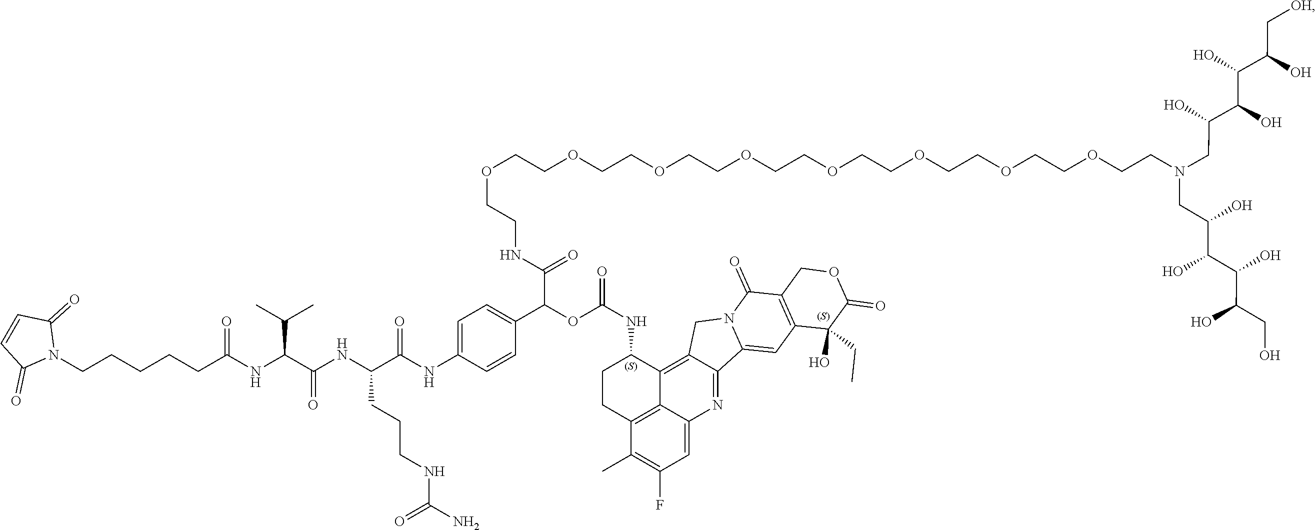

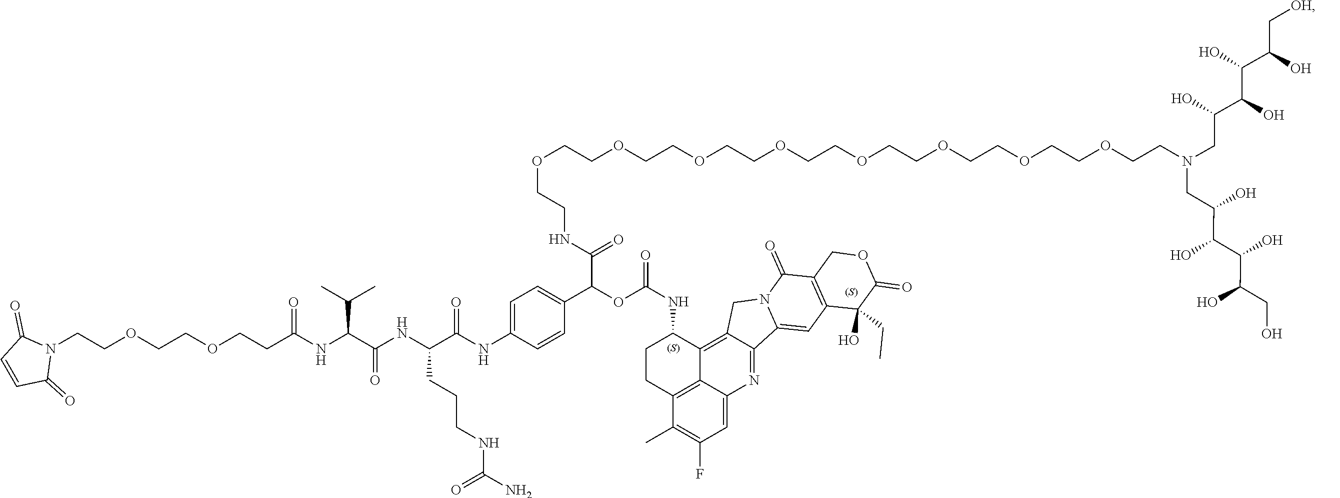

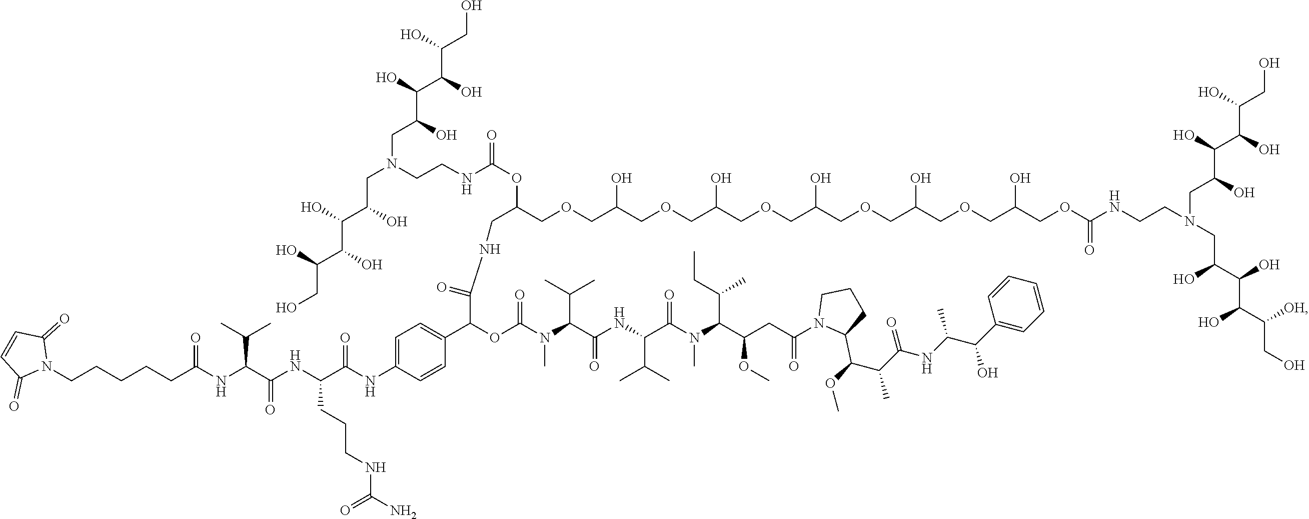 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCC(COCC(O)COCC(O)COCC(O)COCC(O)COCC(O)COC(=O)NCCN(C[C@H](O)[C@@H](O)[C@H](O)[C@H](O)CO)C[C@H](O)[C@@H](O)[C@H](O)[C@H](O)CO)OC(=O)NCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

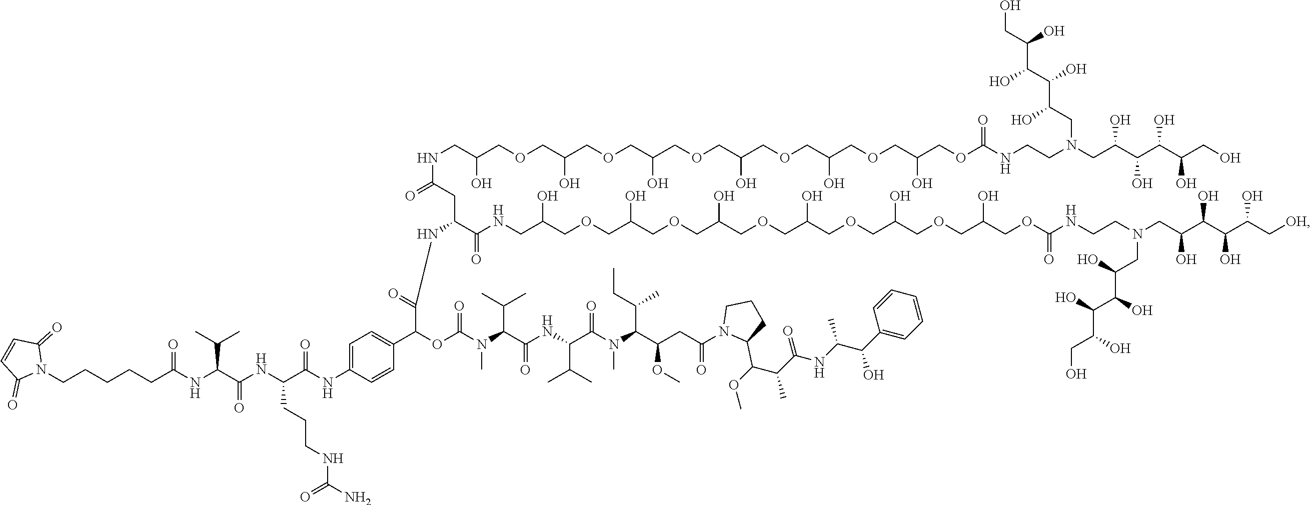 [SMILES: CC[C@H](C)[C@@H]([C@@H](CC(=O)N1CCC[C@H]1C(OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@H](CC(=O)NCC(O)COCC(O)COCC(O)COCC(O)COCC(O)COCC(O)COC(=O)NCCN(C[C@H](O)[C@@H](O)[C@H](O)[C@H](O)CO)C[C@H](O)[C@@H](O)[C@H](O)[C@H](O)CO)C(=O)NCC(O)COCC(O)COCC(O)COCC(O)COCC(O)COCC(O)COC(=O)NCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

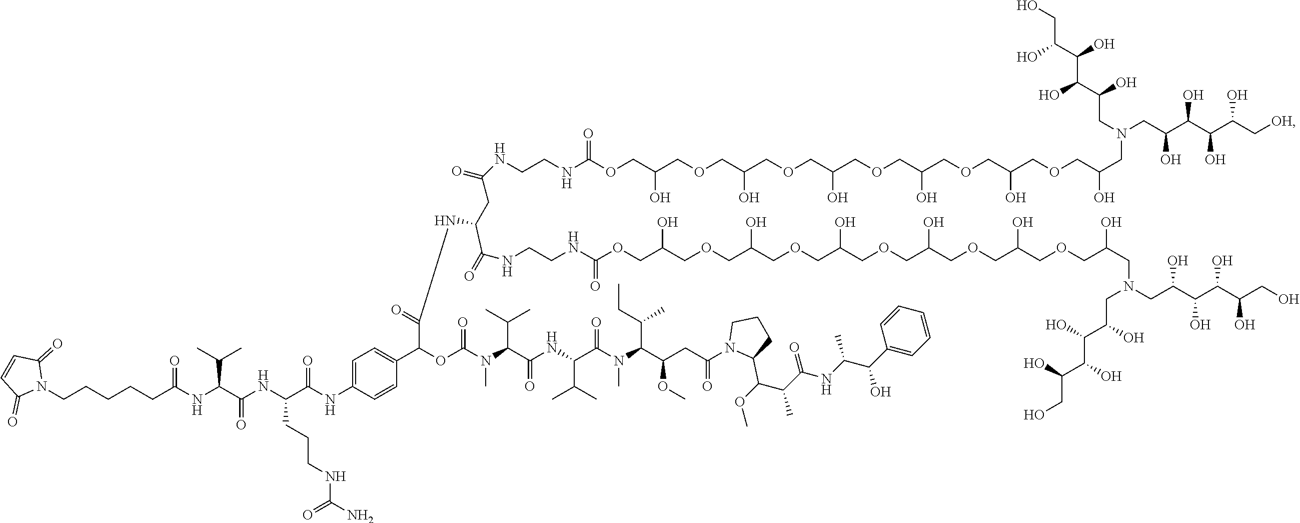 [SMILES: CC[C@H](C)[C@@H]([C@@H](CC(=O)N1CCC[C@H]1C(OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@H](CC(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

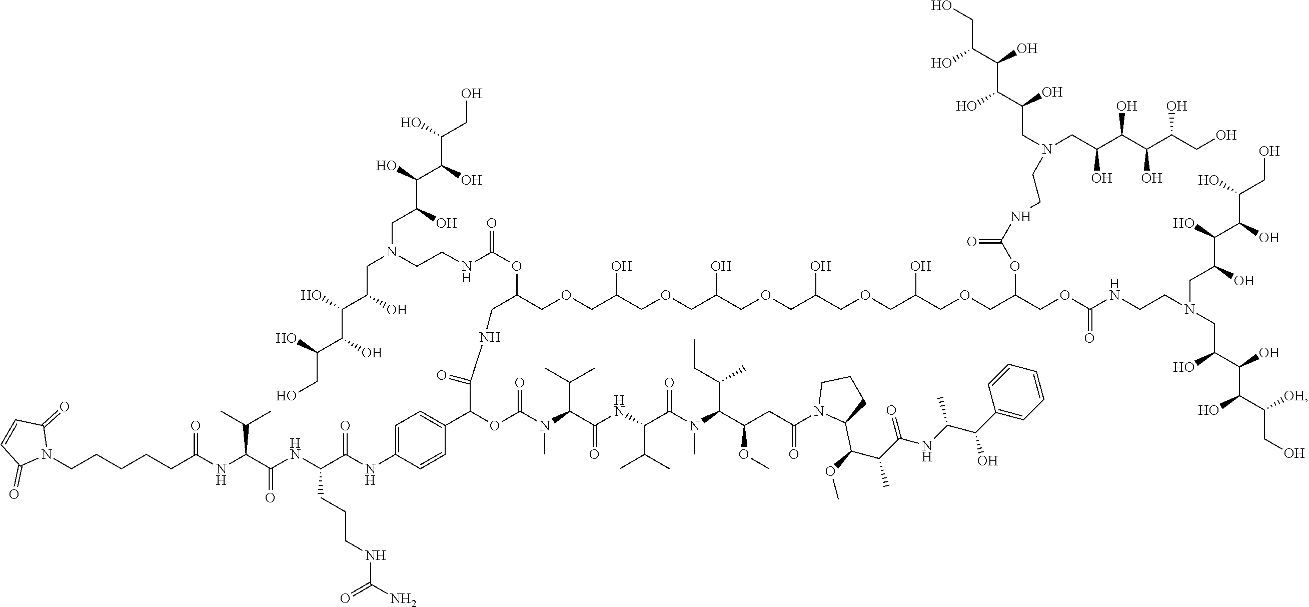

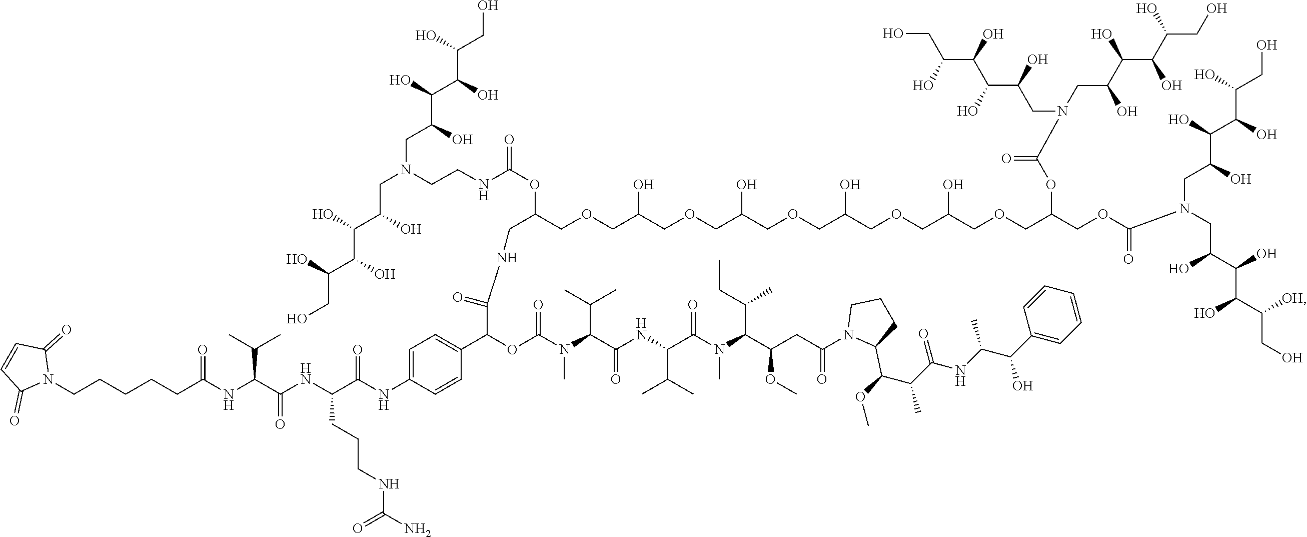

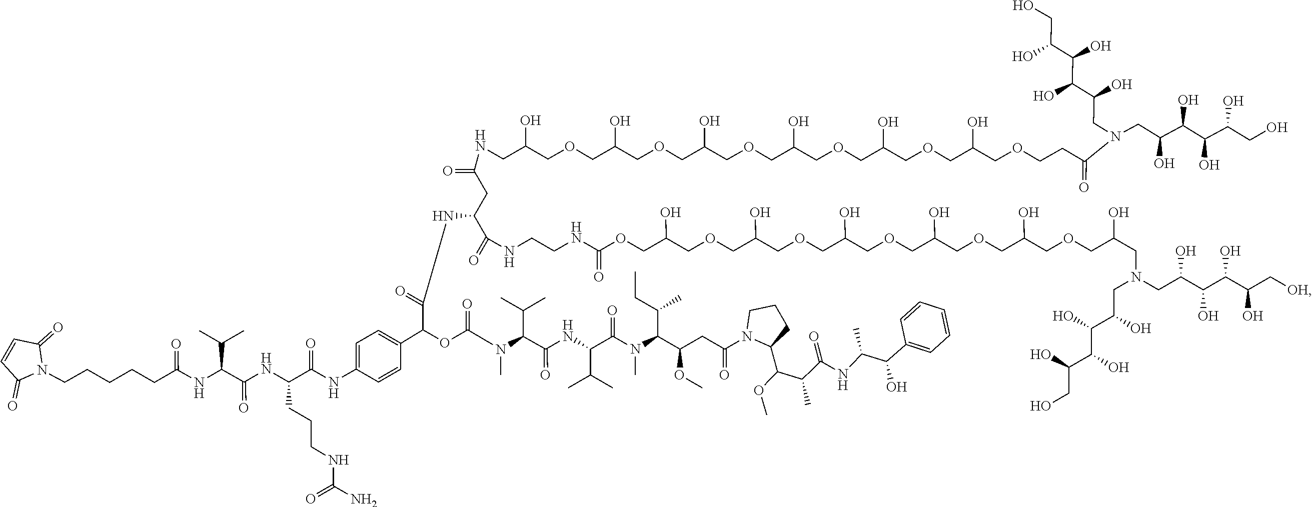 [SMILES: CC[C@H](C)[C@@H]([C@@H](CC(=O)N1CCC[C@H]1C(OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@H](CC(=O)NCC(O)COCC(O)COCC(O)COCC(O)COCC(O)COCC(O)COCCC(=O)N(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

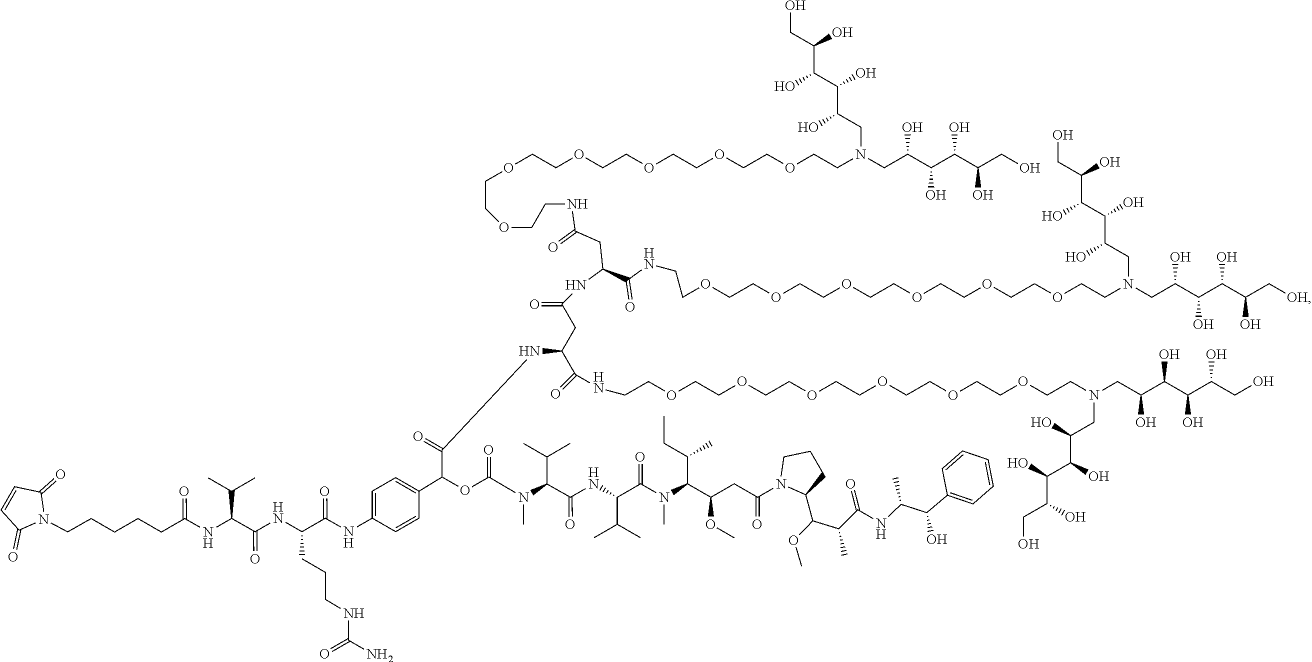

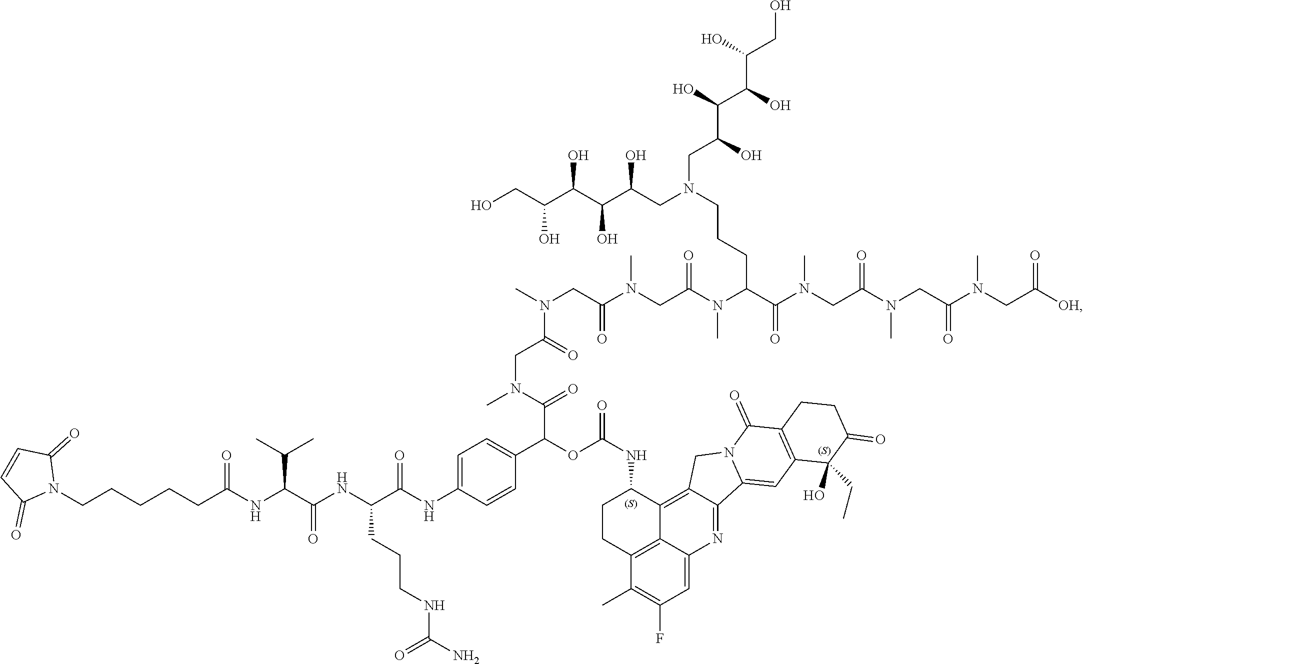

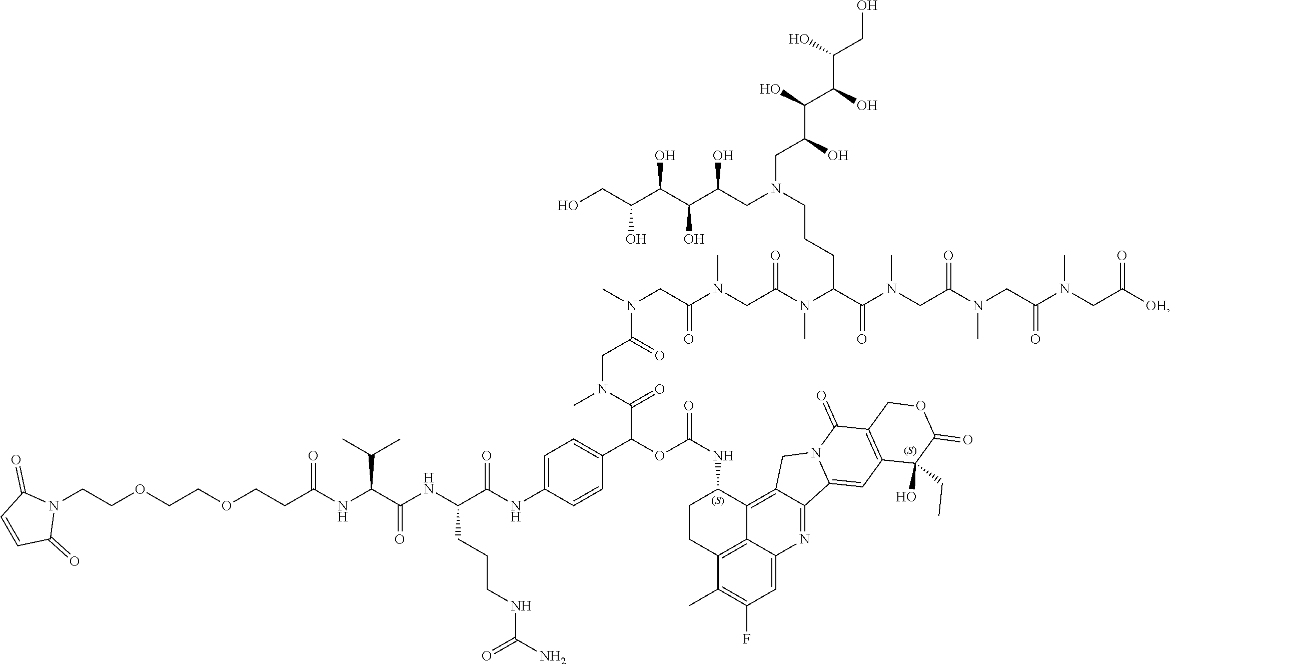

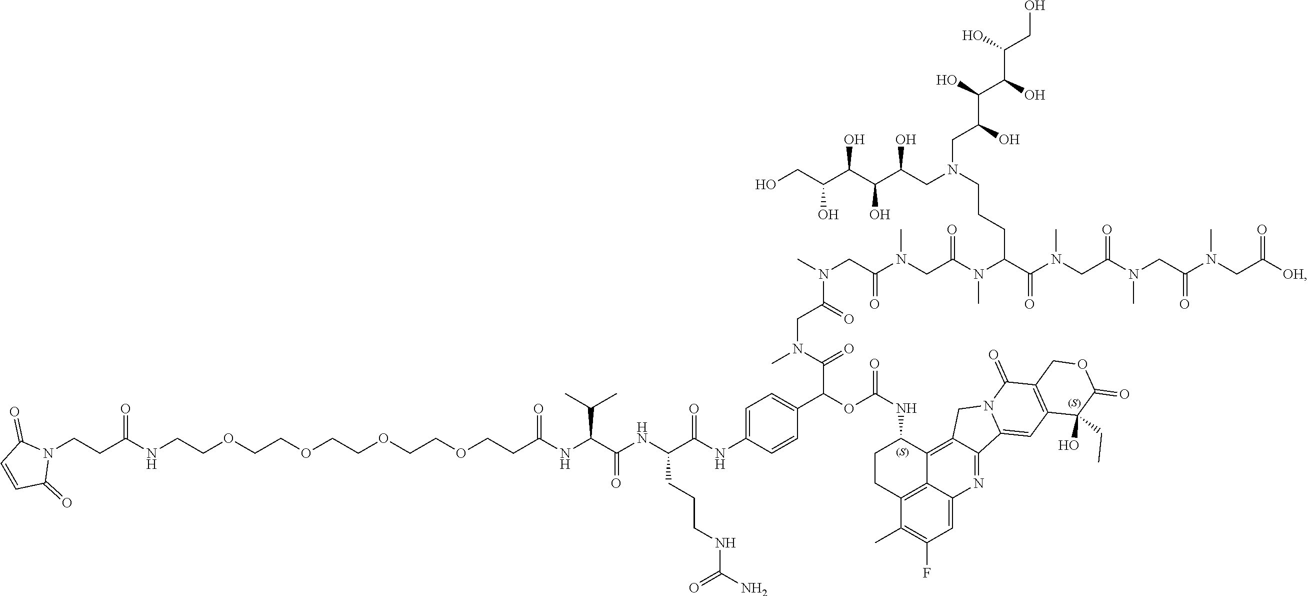

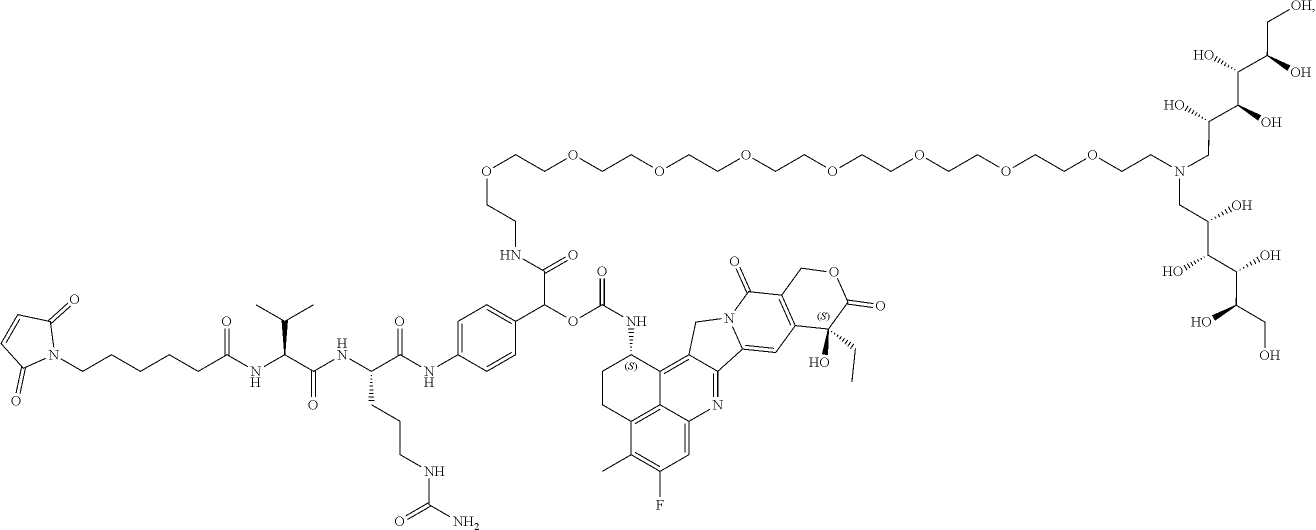

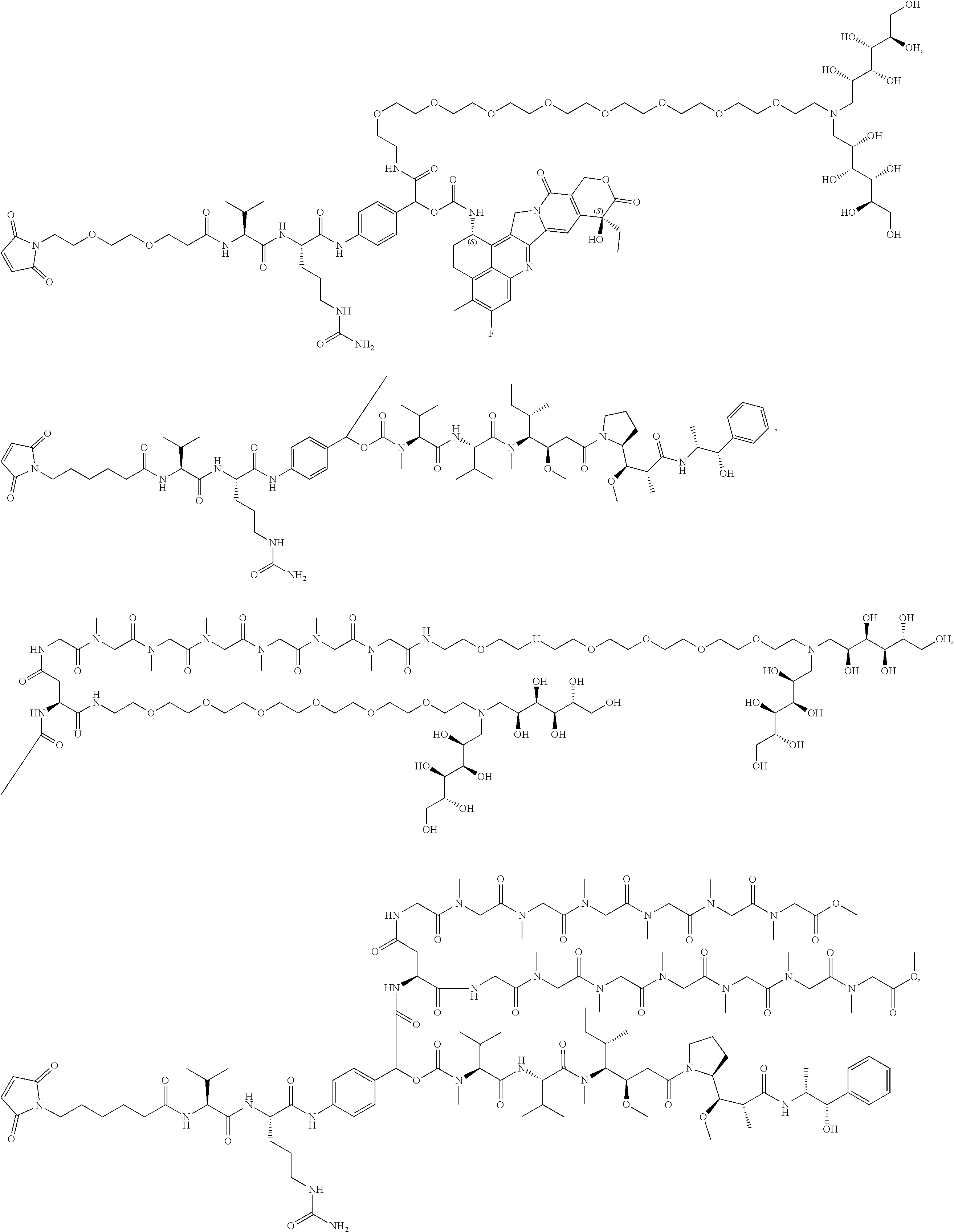

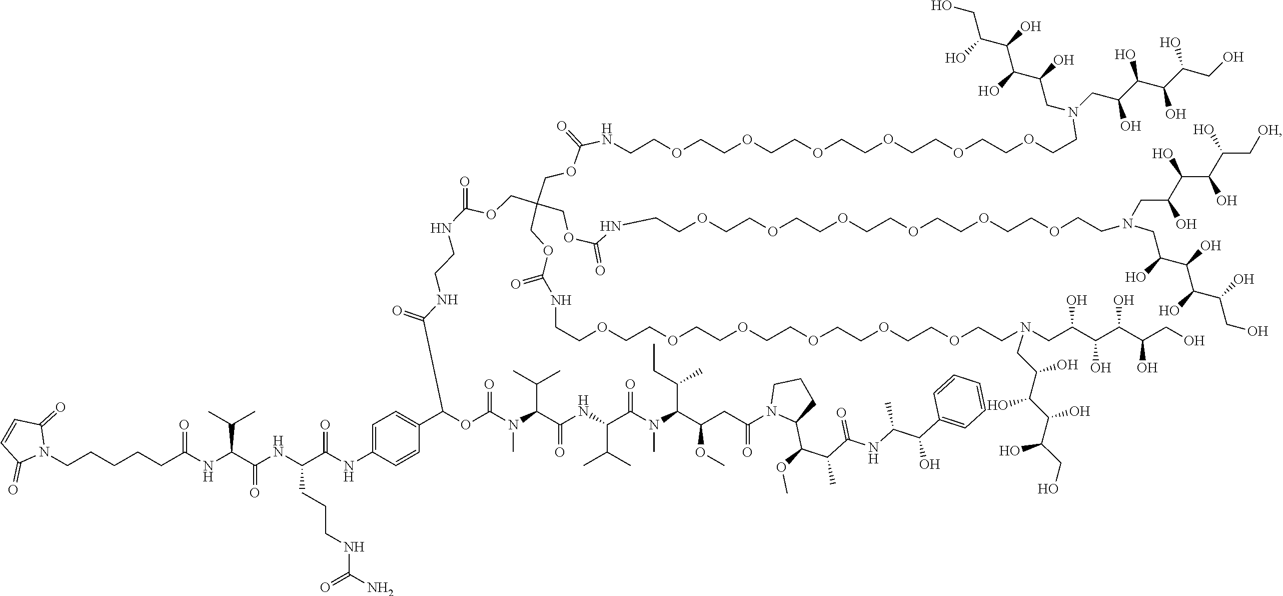

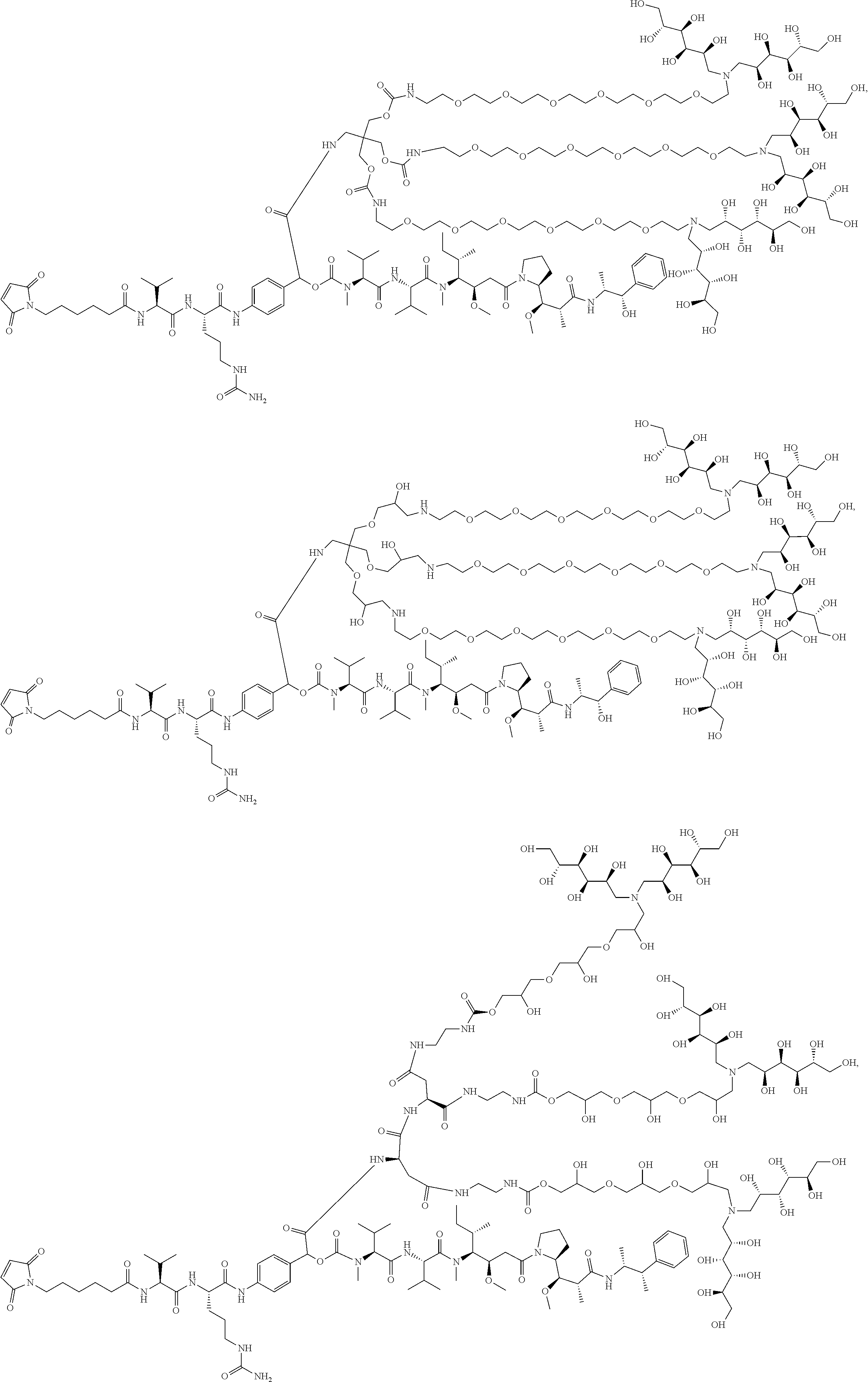

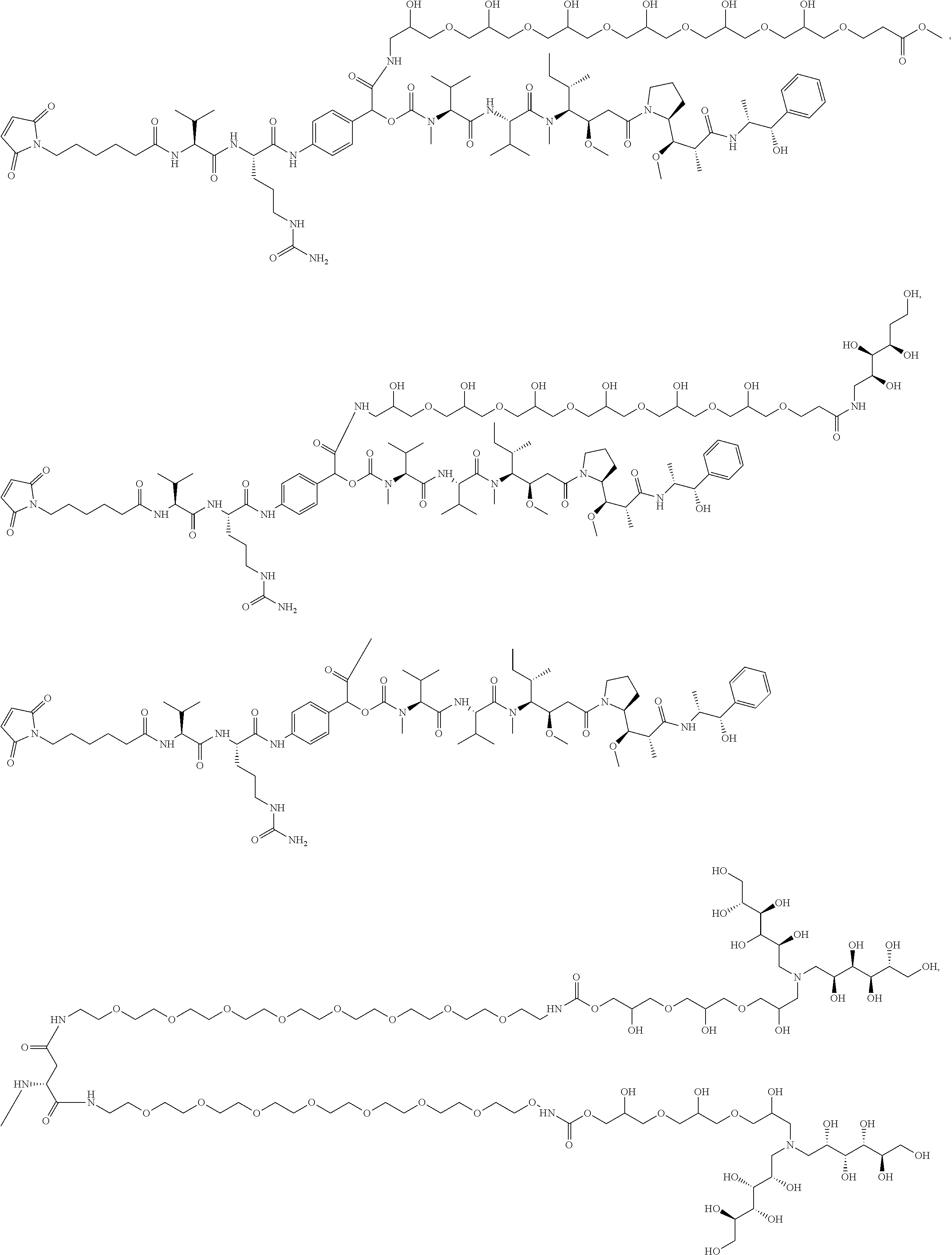

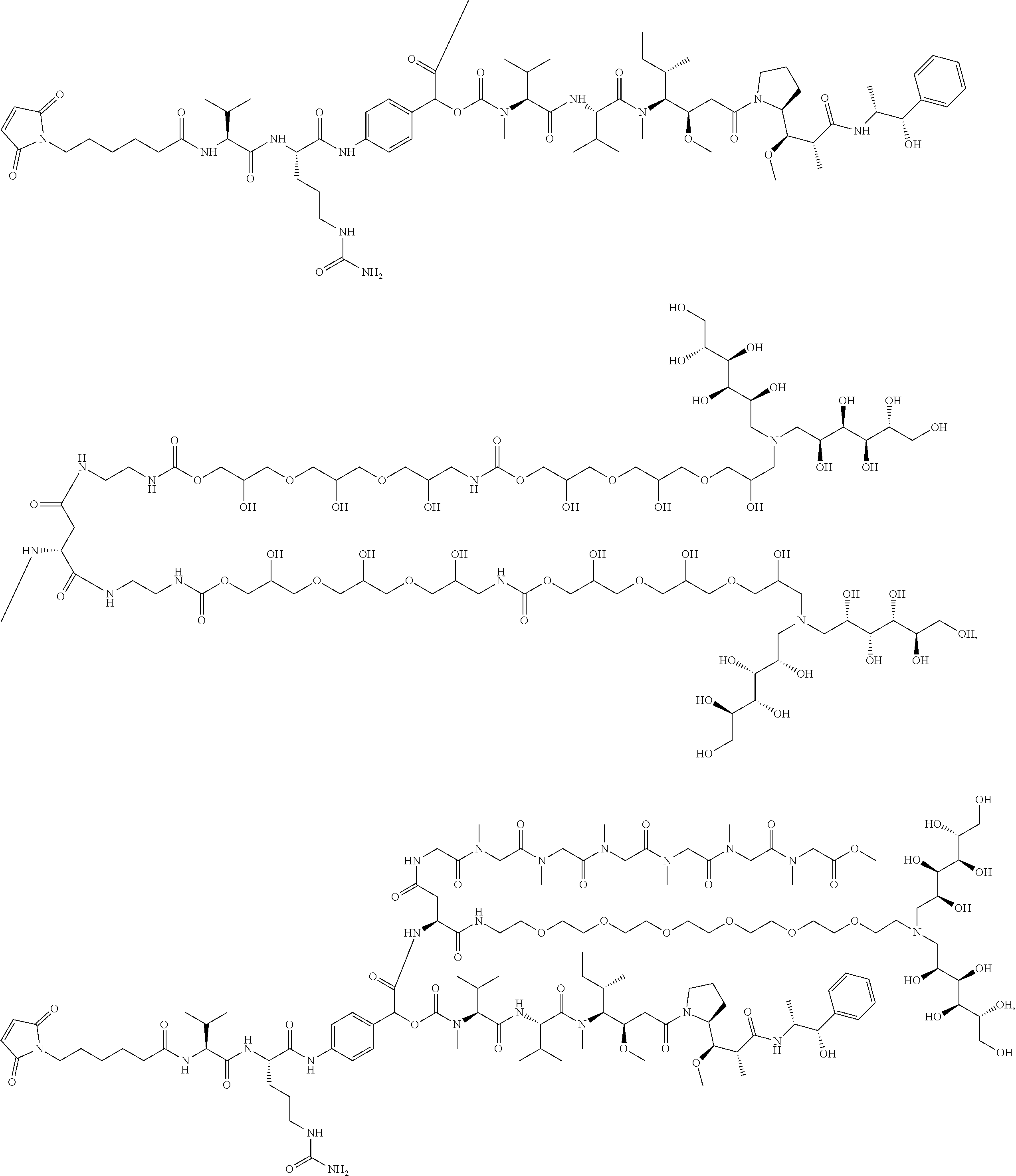

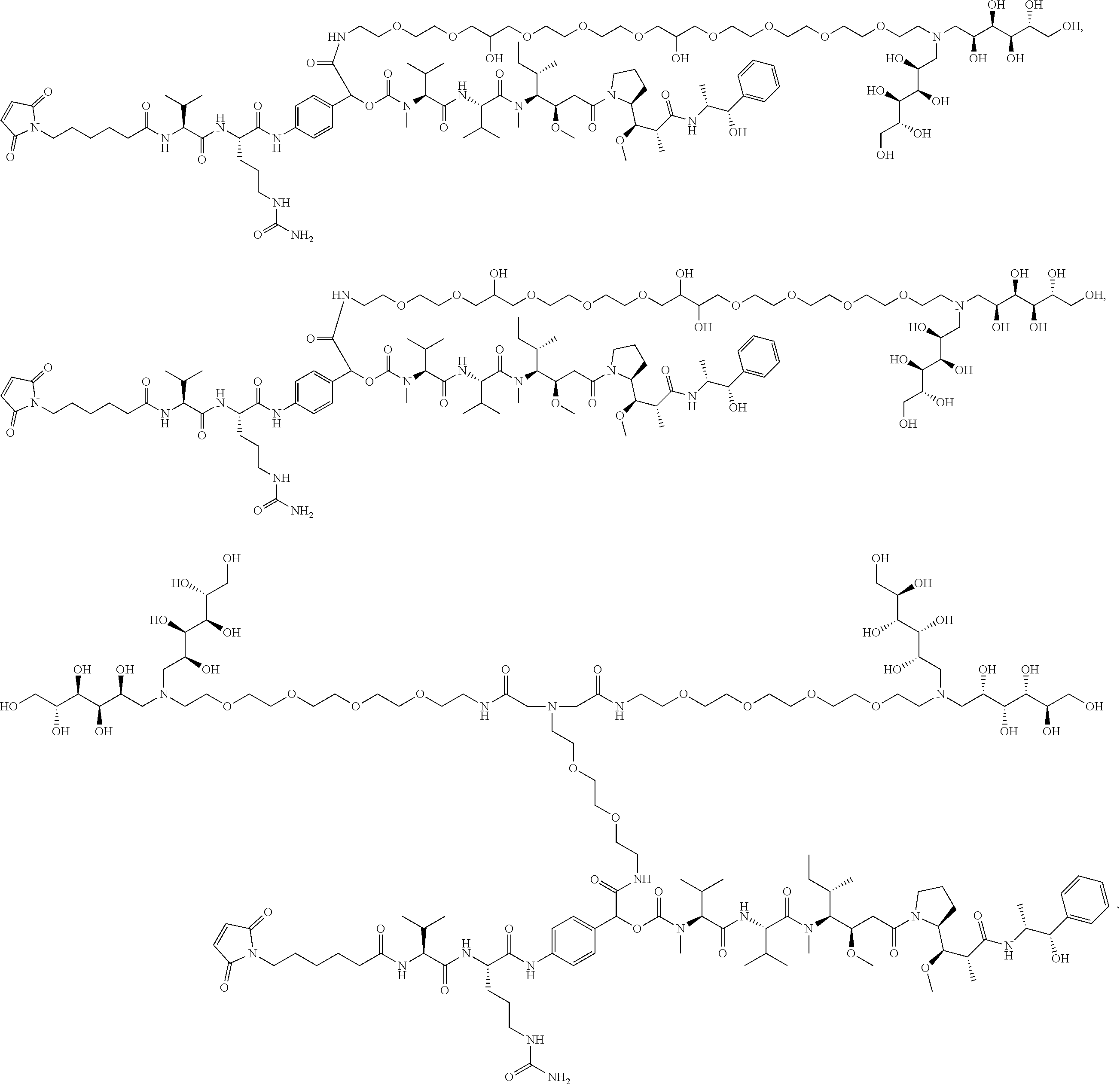

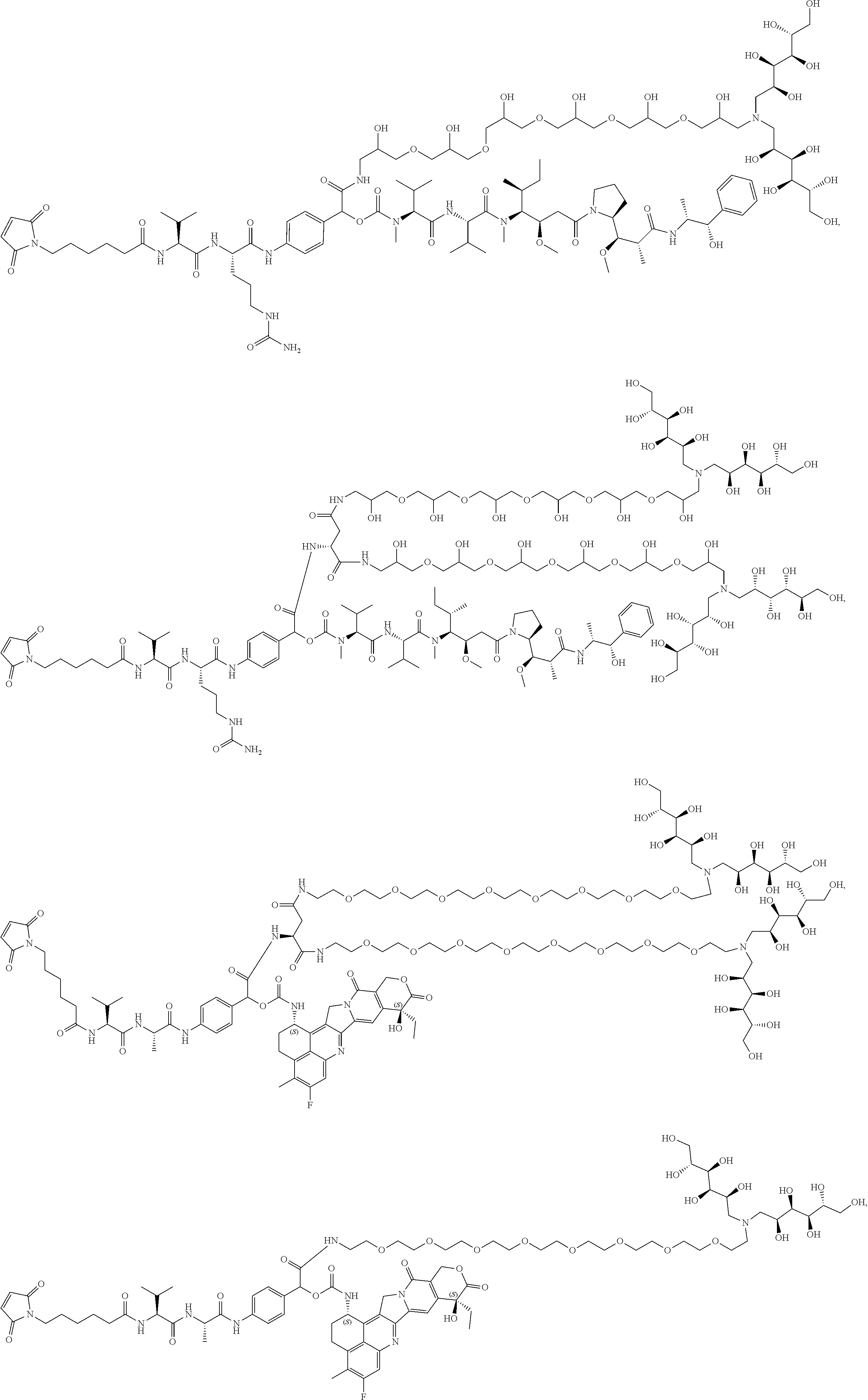

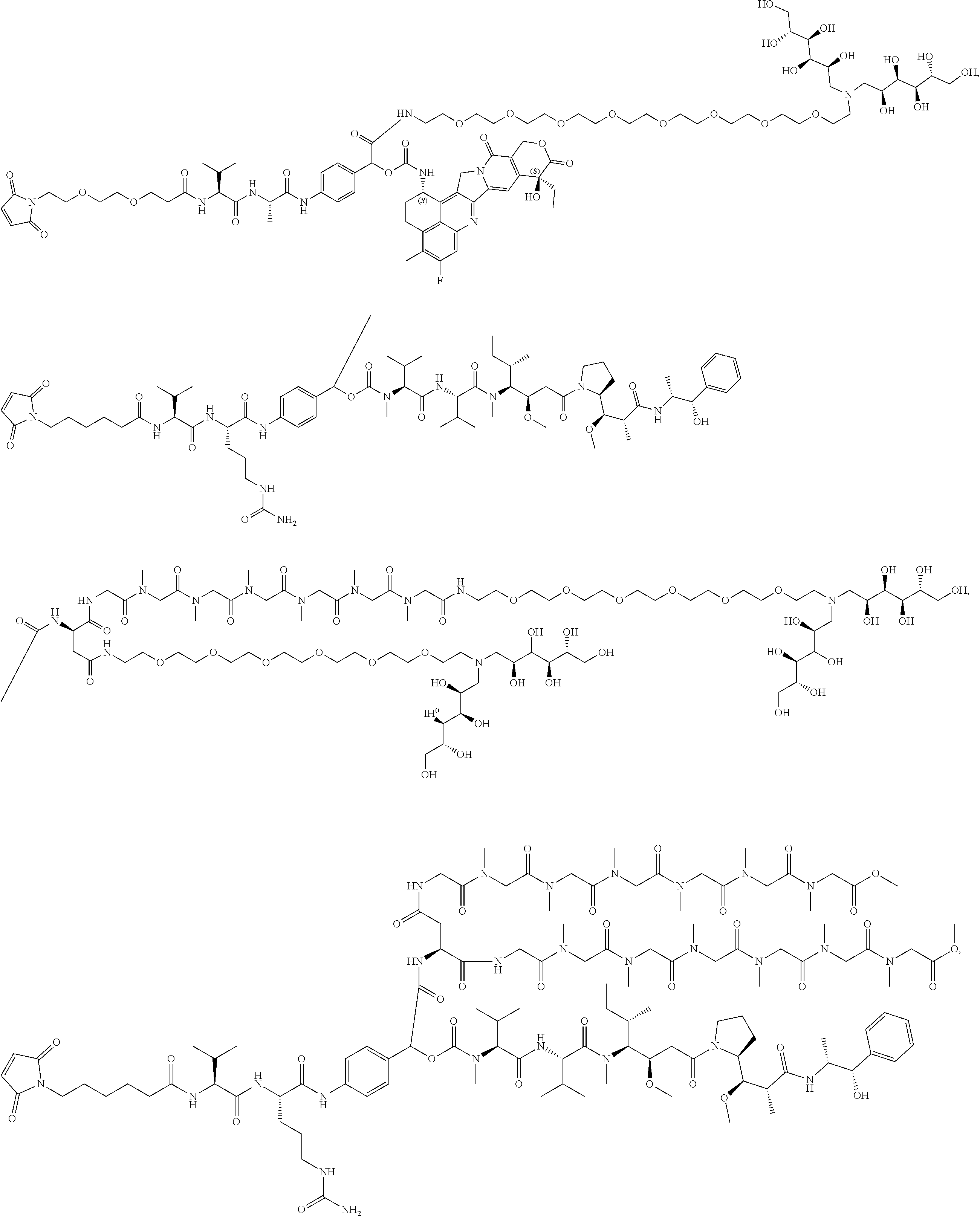

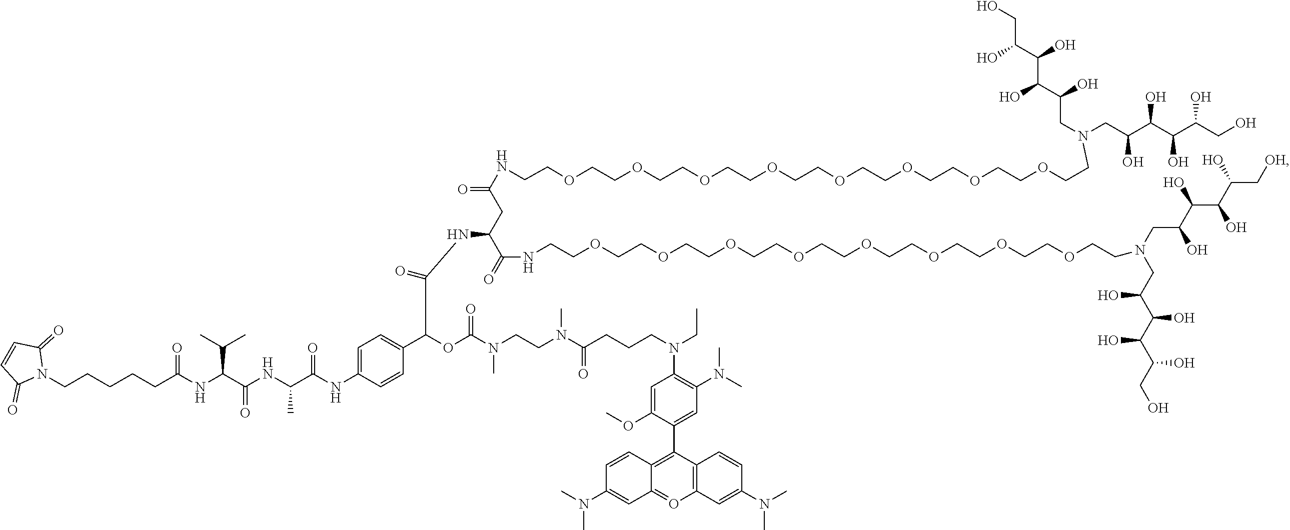

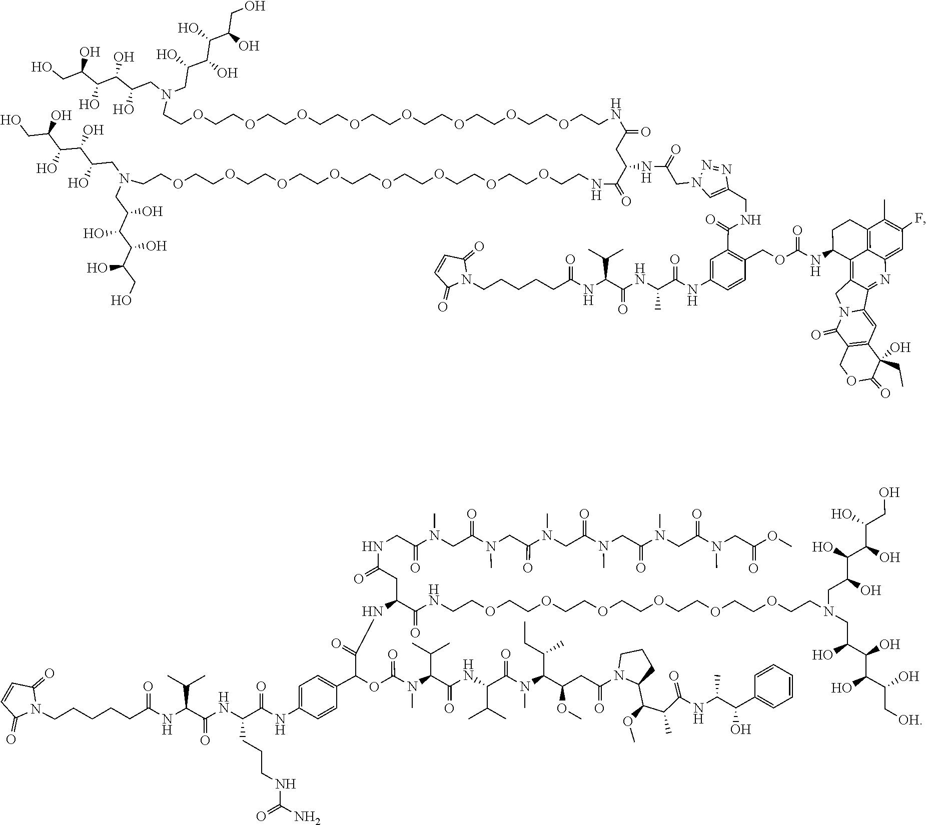

102. A conjugate comprising a Targeting group attached to the Drug-linker of any one of claims 78 to 101, wherein the Targeting group specifically binds to the target molecule.

103. The conjugate of claim 102, wherein the Targeting group is selected from an antibody or an antigen-binding portion thereof.

104. The conjugate of claim 103, wherein the Targeting group is a monoclonal antibody, a Fab, a Fab′, an F(ab′), an Fv, a disulfide linked Fc, a scFv, a single domain antibody, a diabody, a bi-specific antibody, or a multi-specific antibody.

105. The conjugate of claim 102, wherein the Targeting group is a diabody, a DART, an anticalin, an affibody, an avimer, a DARPin, or an adnectin.

106. The conjugate of any one of claims 102 to 105, wherein the Targeting group is mono-specific.

107. The conjugate of any one of claims 102 to 106, wherein the Targeting group is bivalent.

108. The conjugate of any one of claims 102 to 105, wherein the Targeting group is bispecific.

109. The conjugate of any one of claims 102 to 108, wherein the average drug loading (p^{load}) of the conjugate is from about 1 to about 8, about 2, about 4, about 6, about 8, about 10, about 12, about 14, about 16, about 3 to about 5, about 6 to about 8, or about 8 to about 16.

110. The conjugate of any one of claims 107-109, selected from the following: 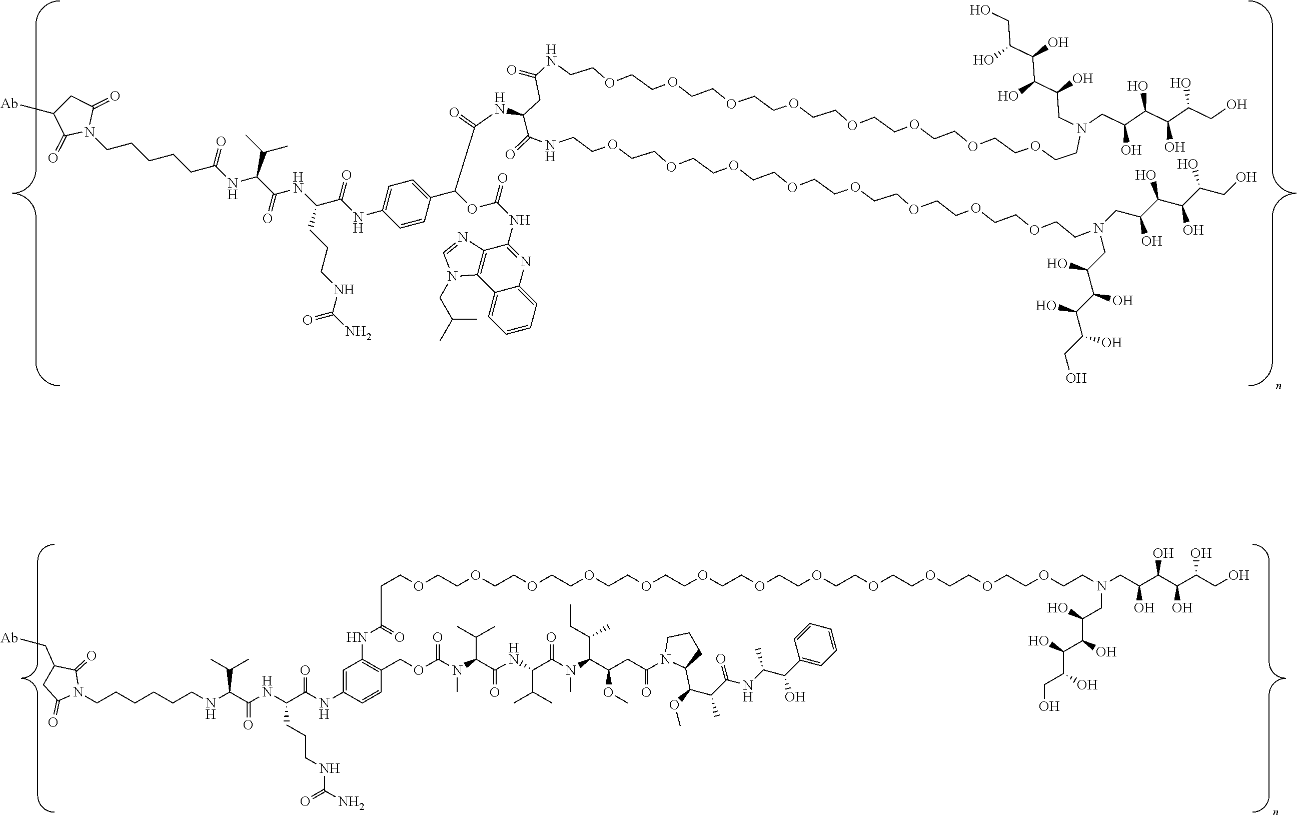 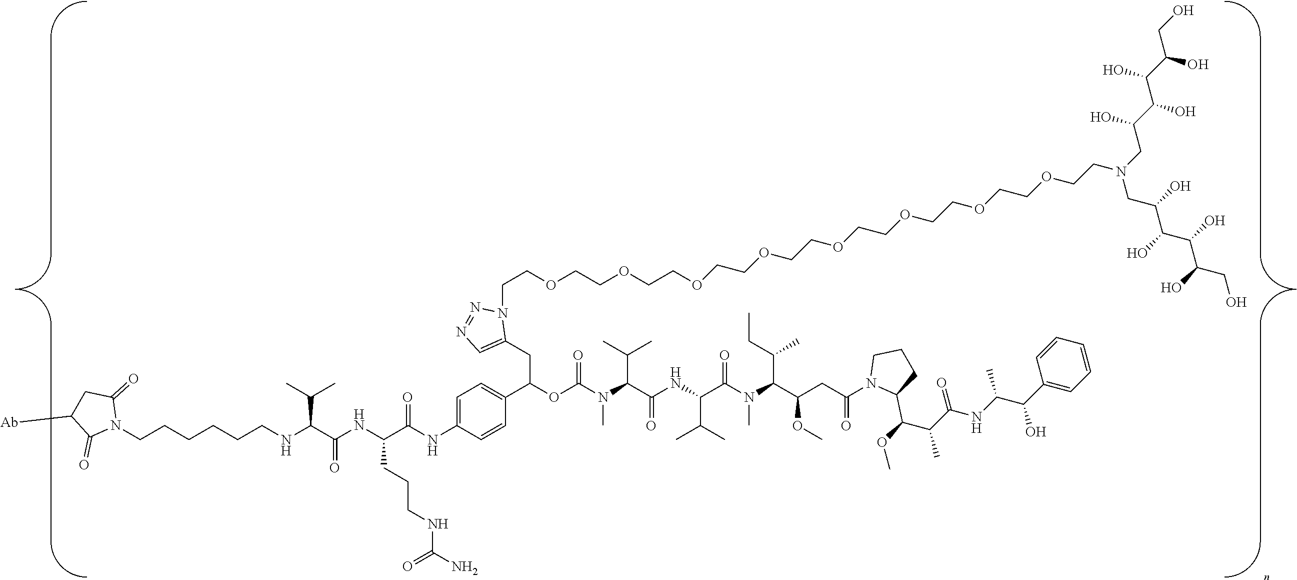 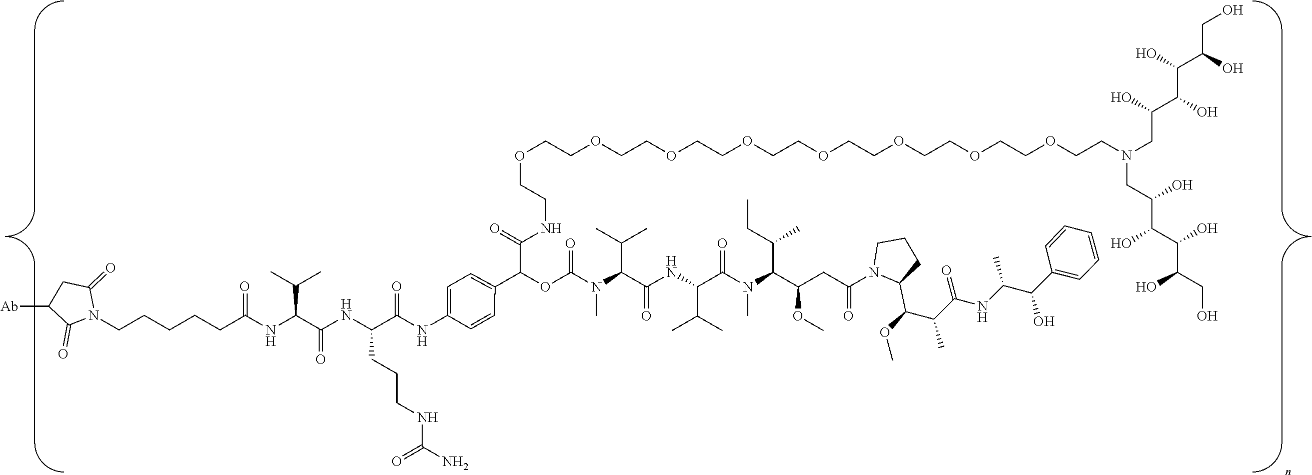 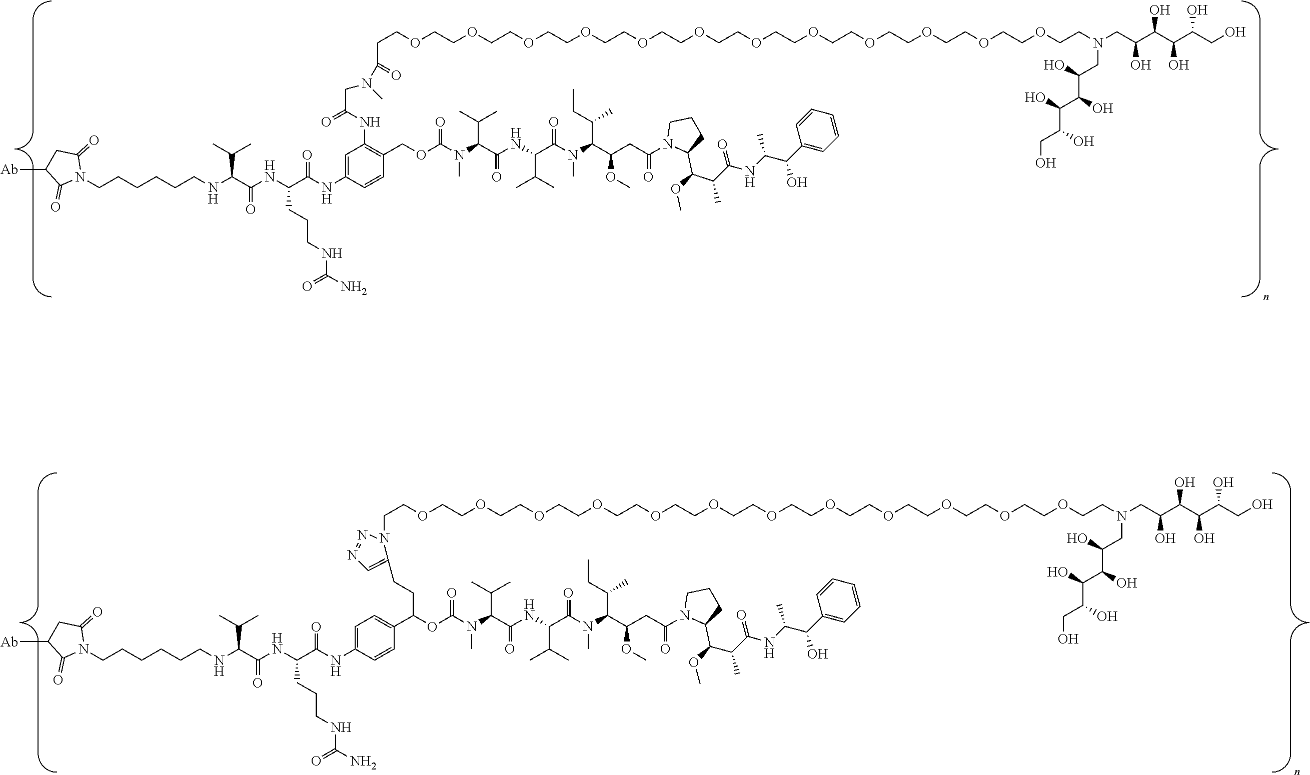 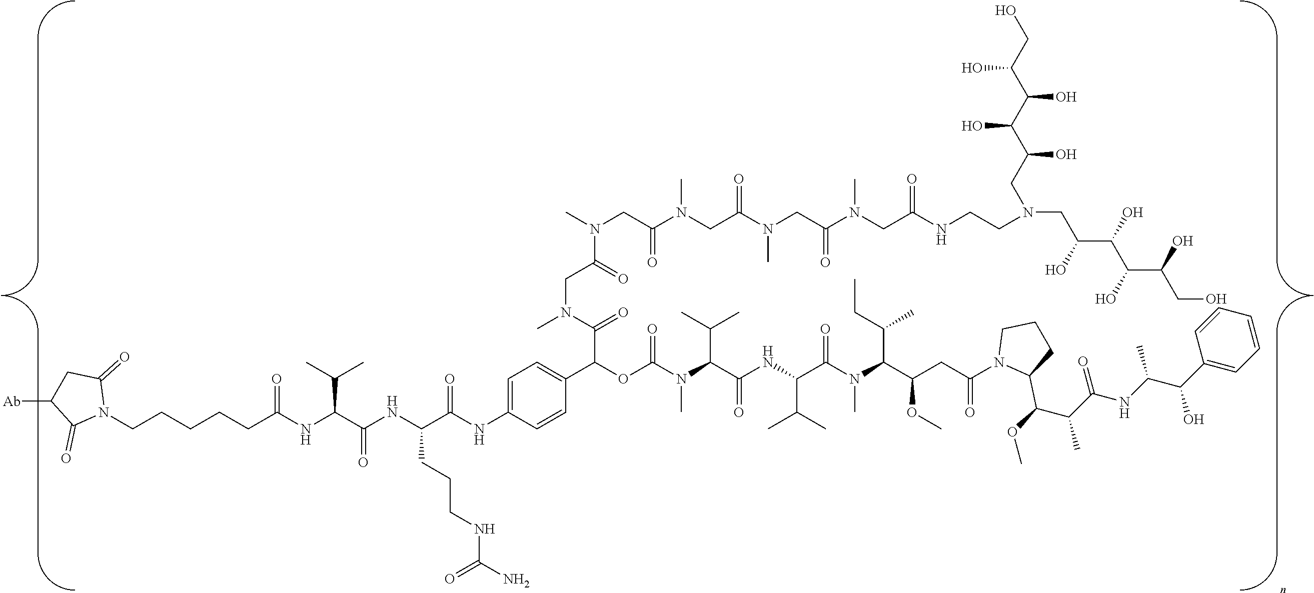 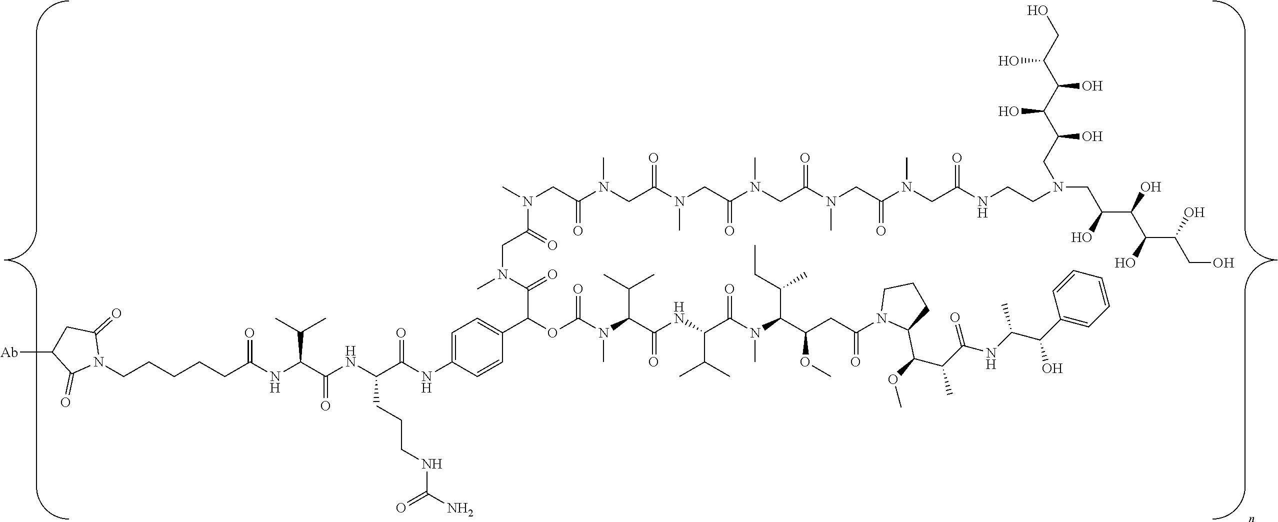 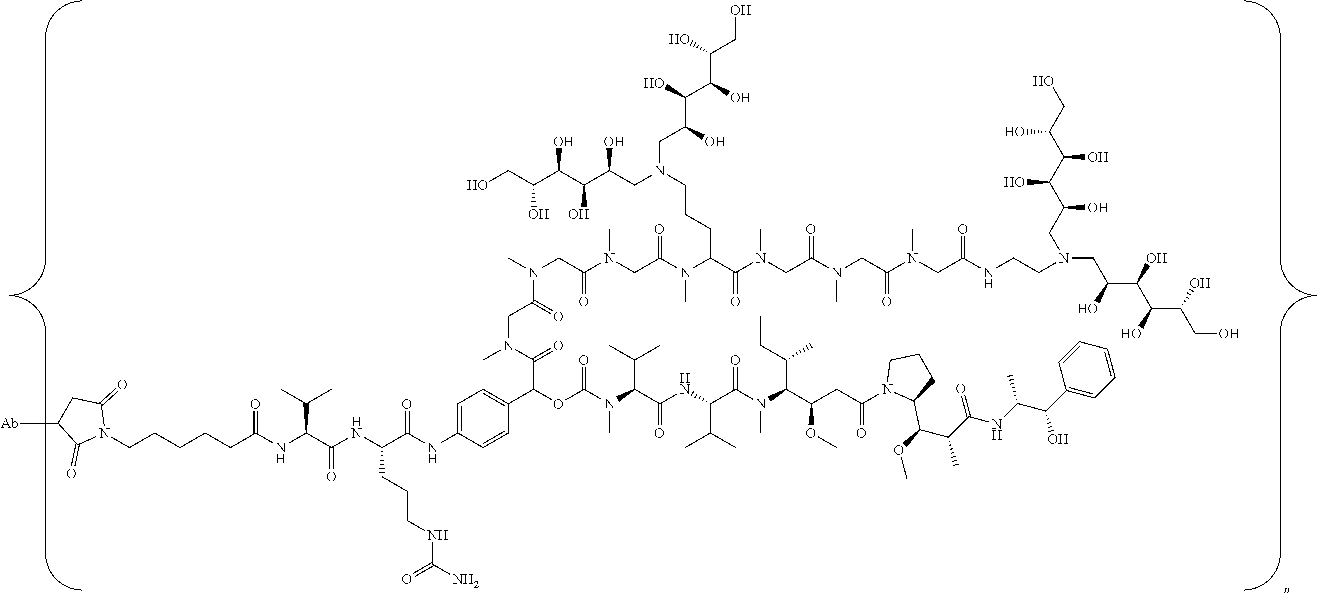 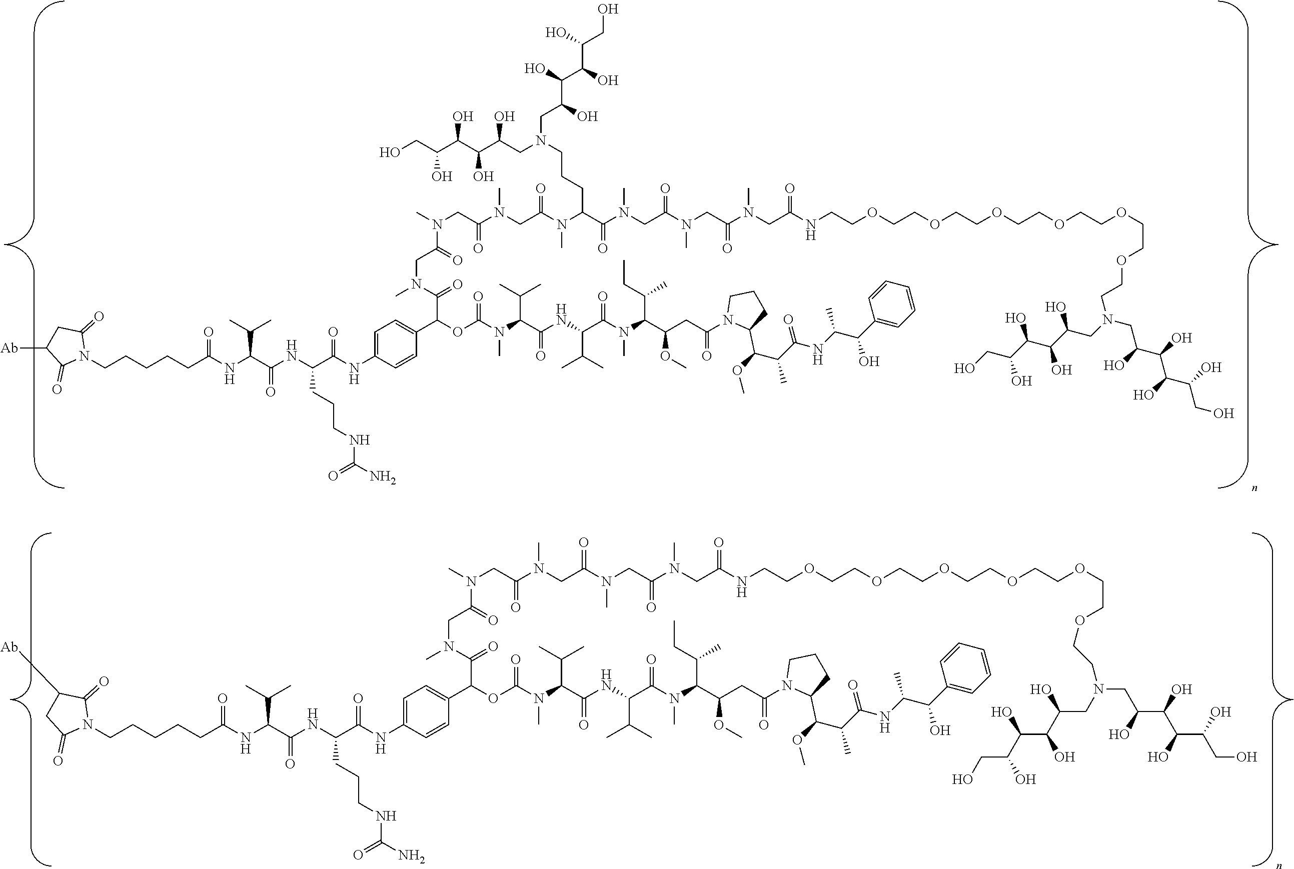 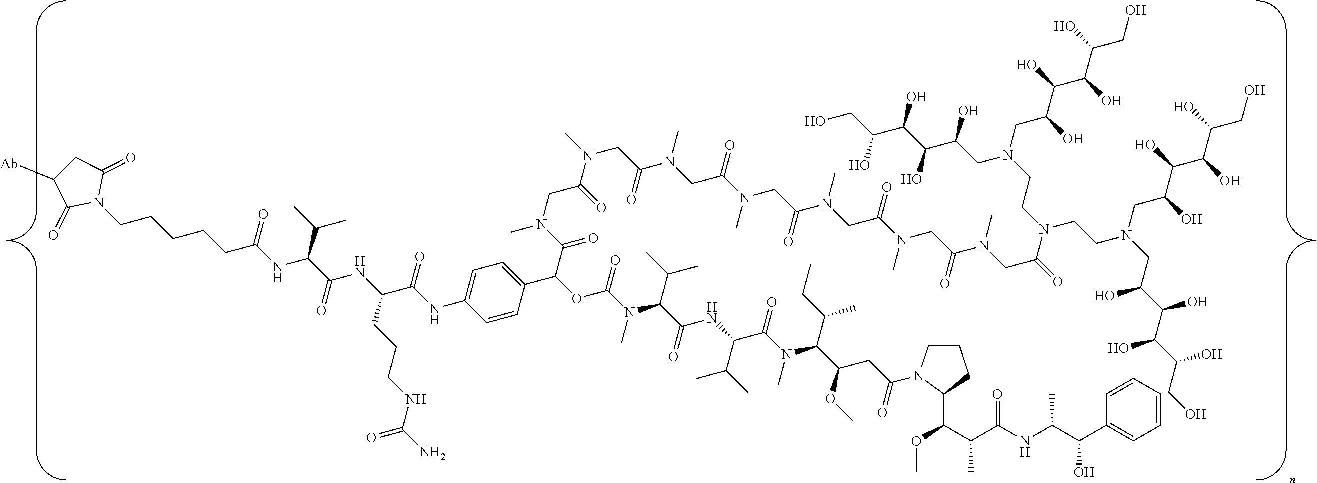 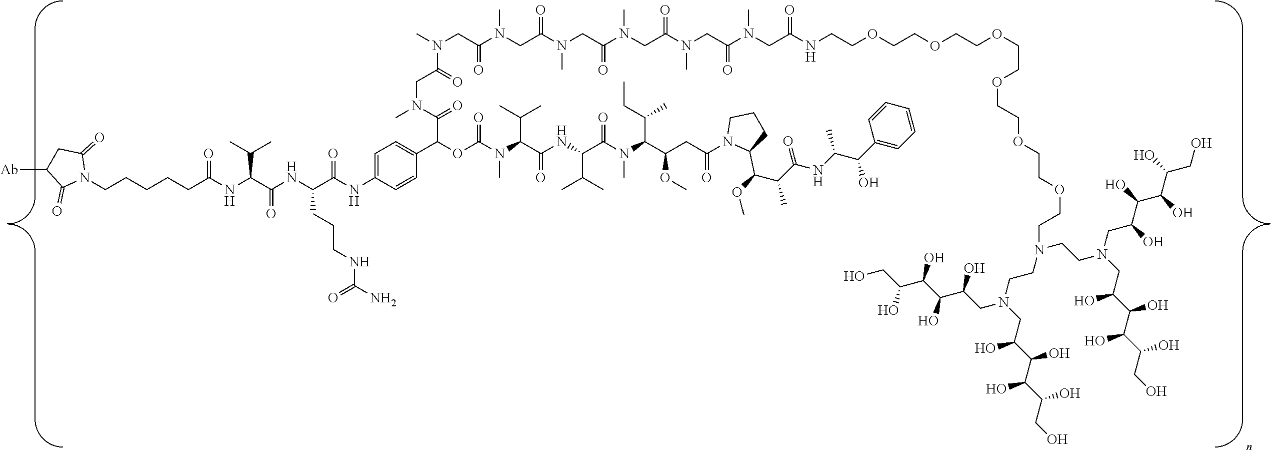 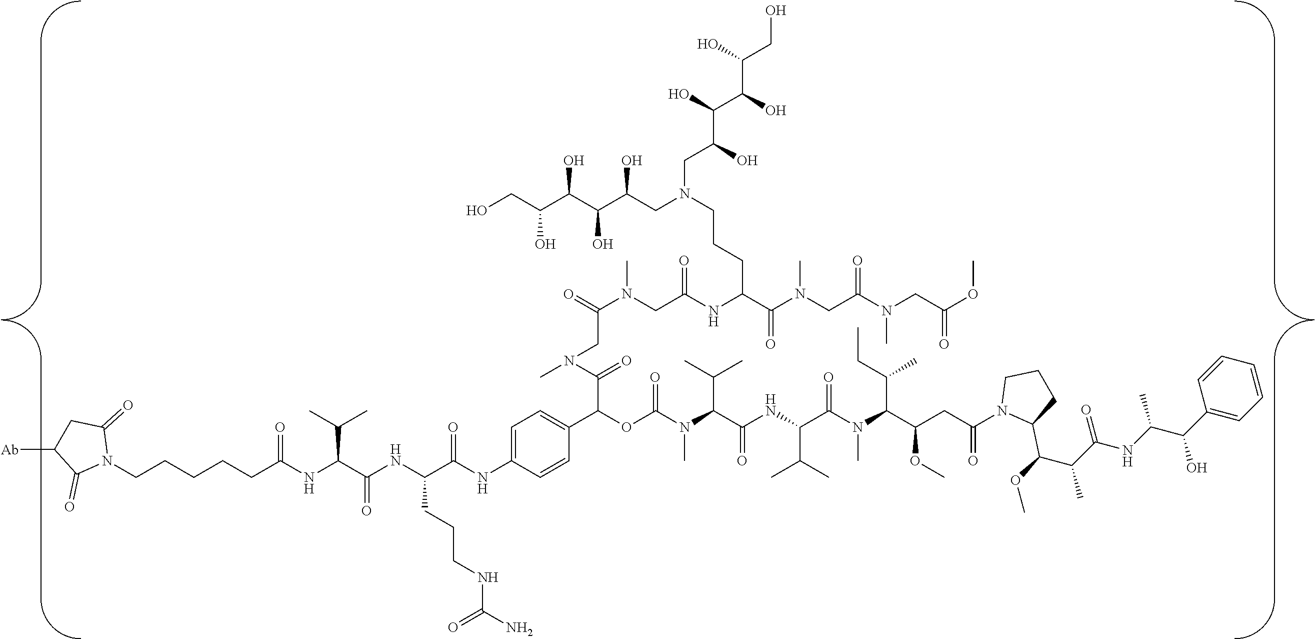 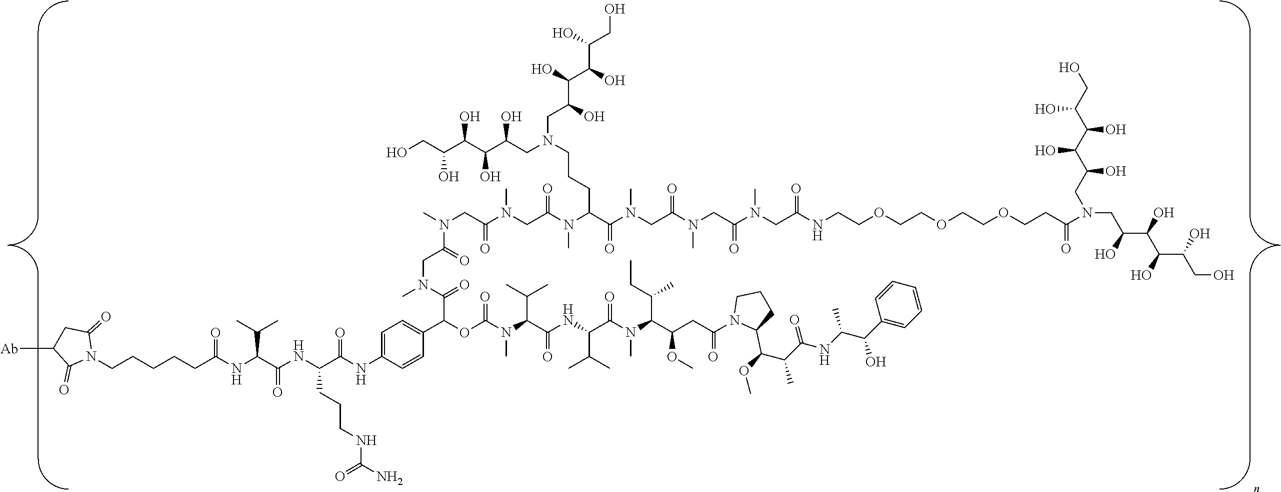 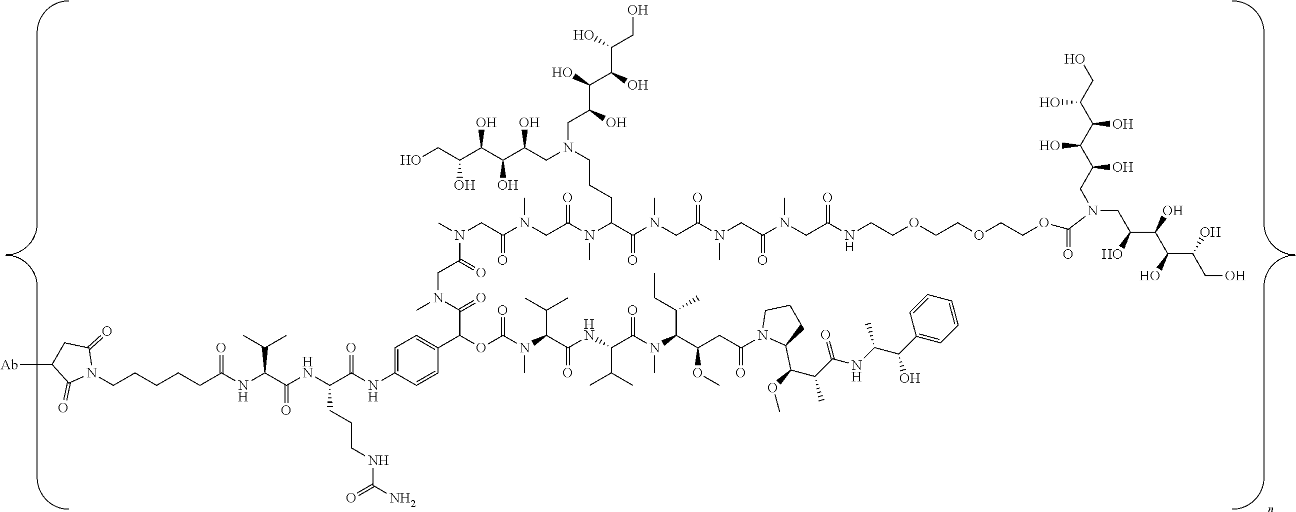 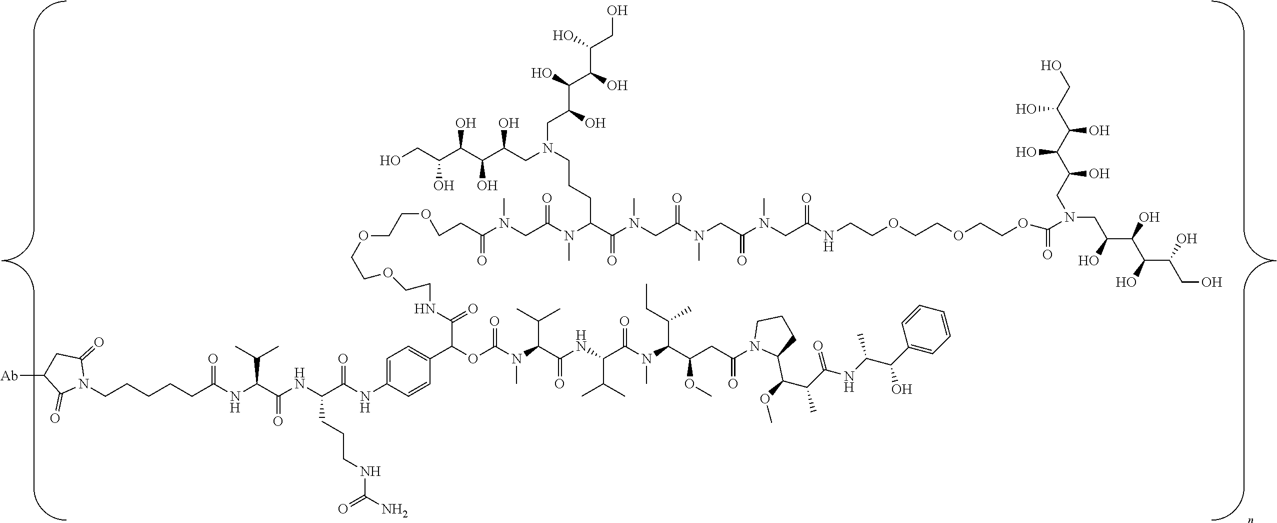 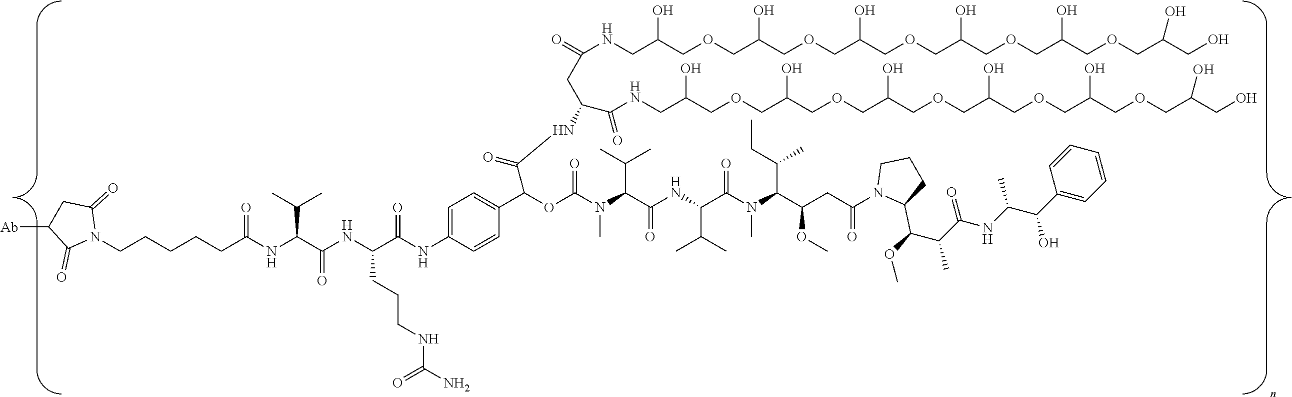 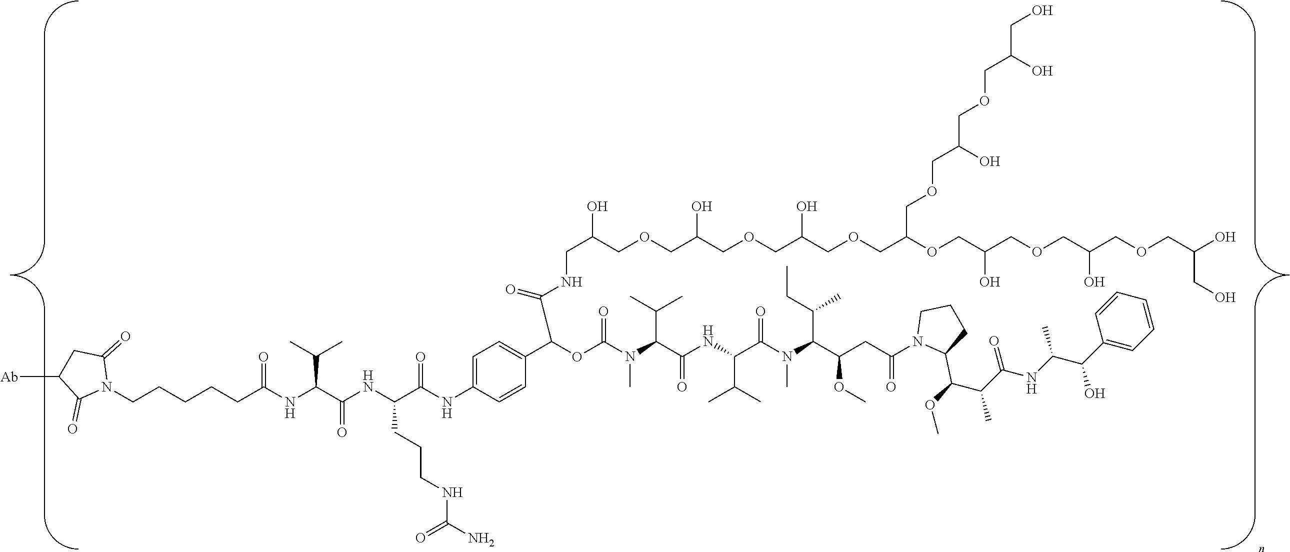 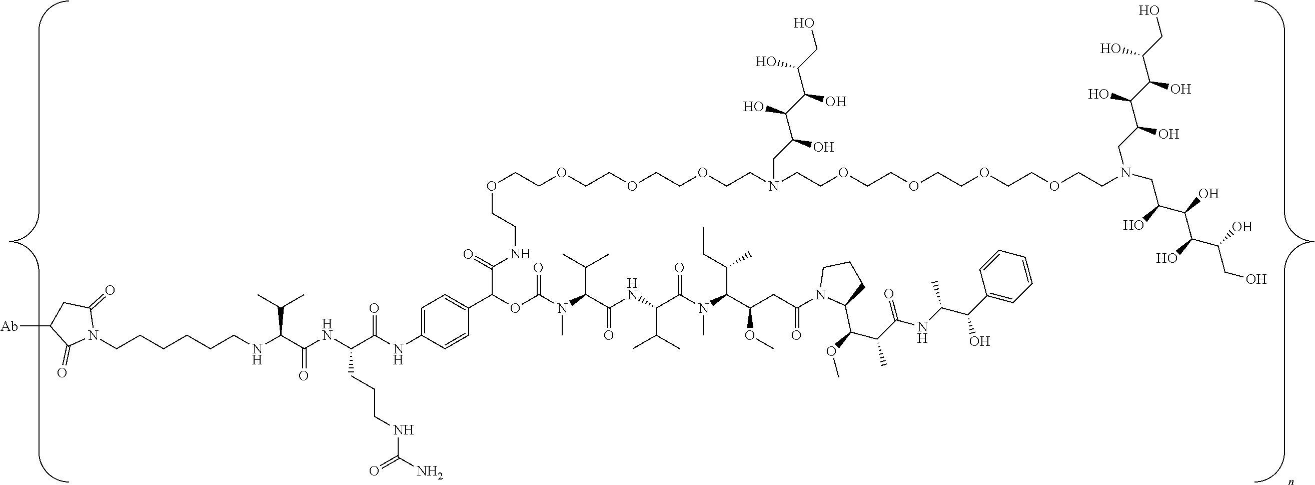 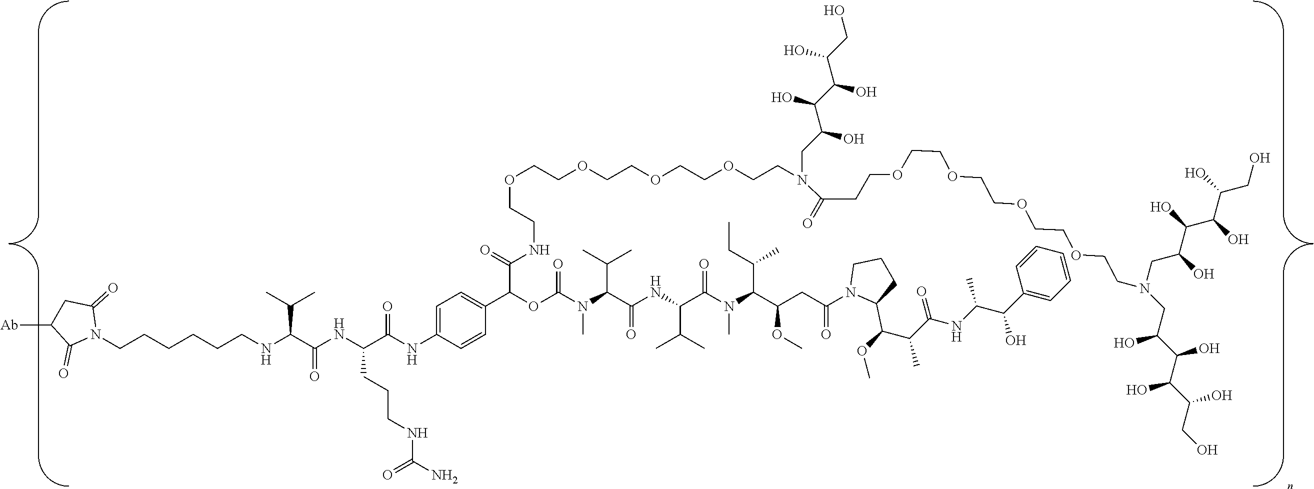 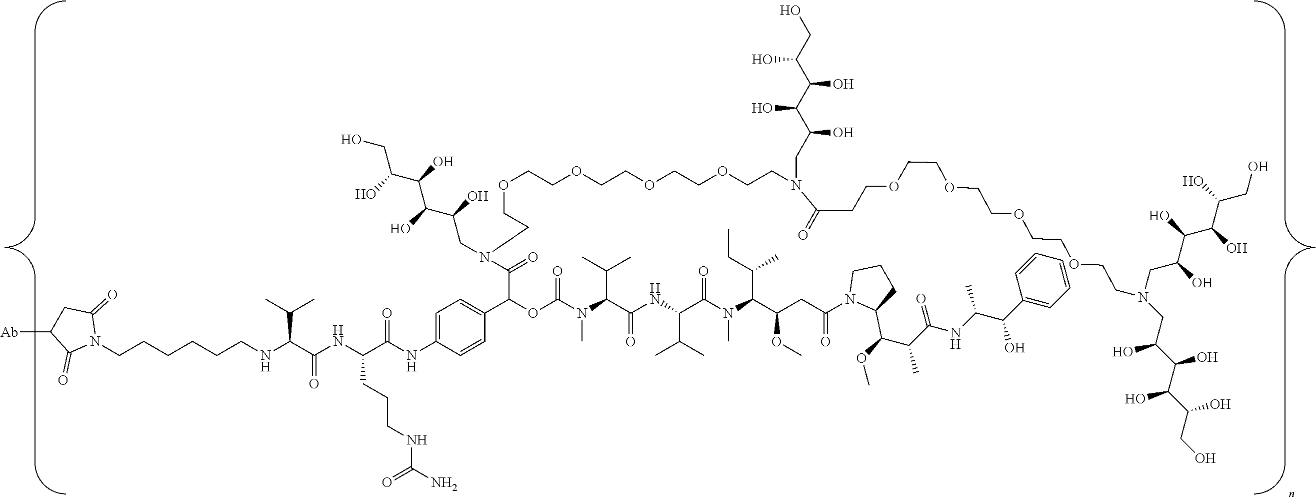 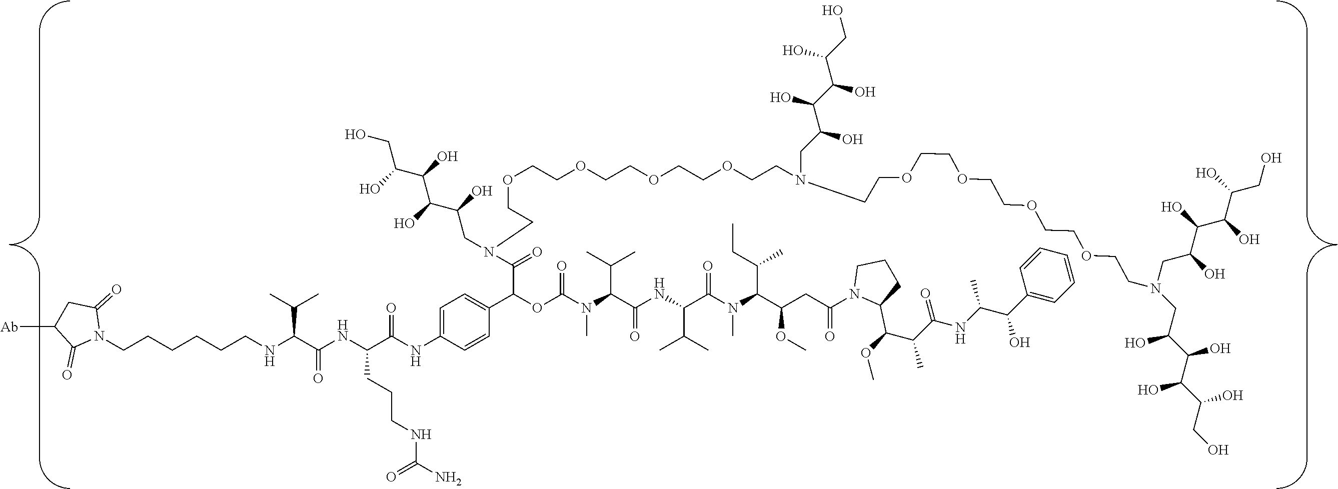 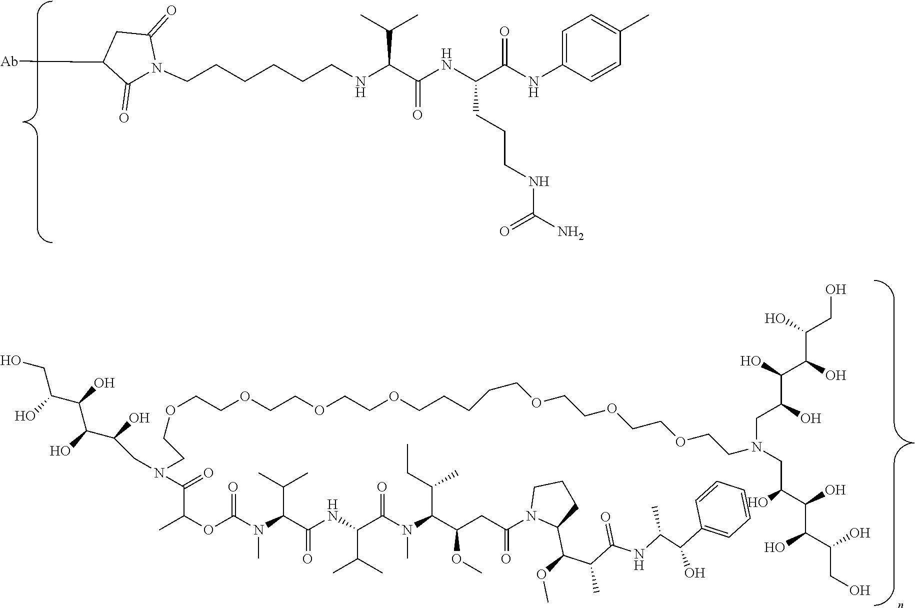 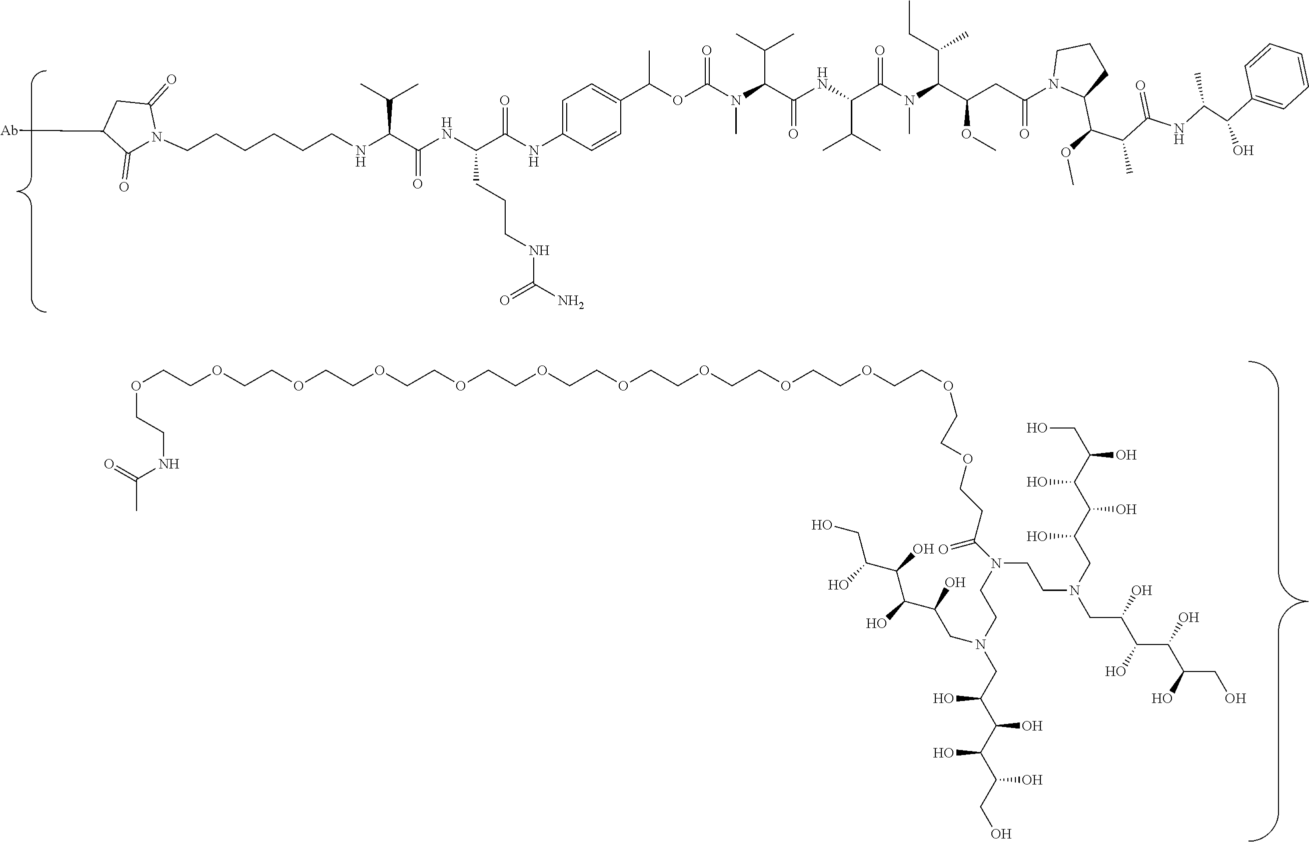 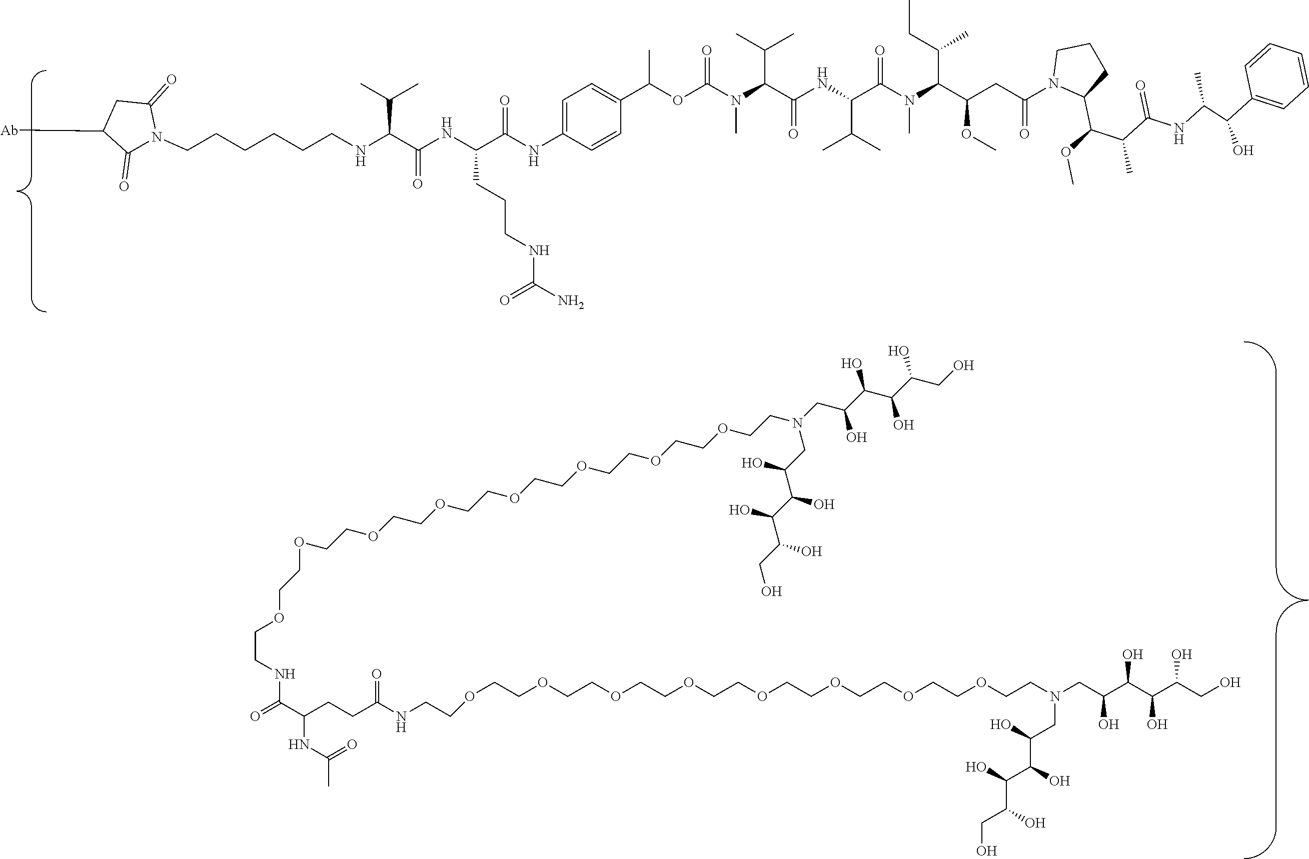 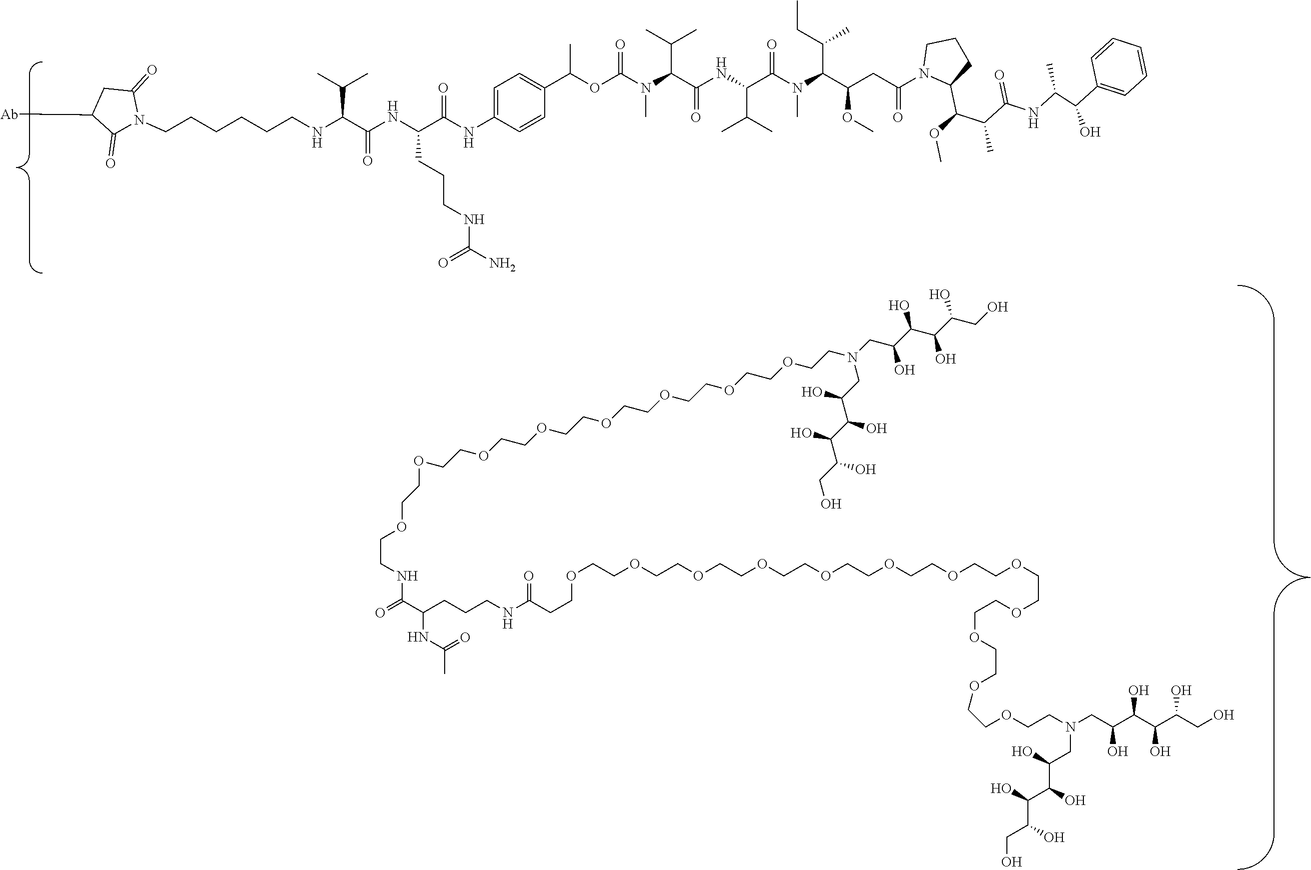 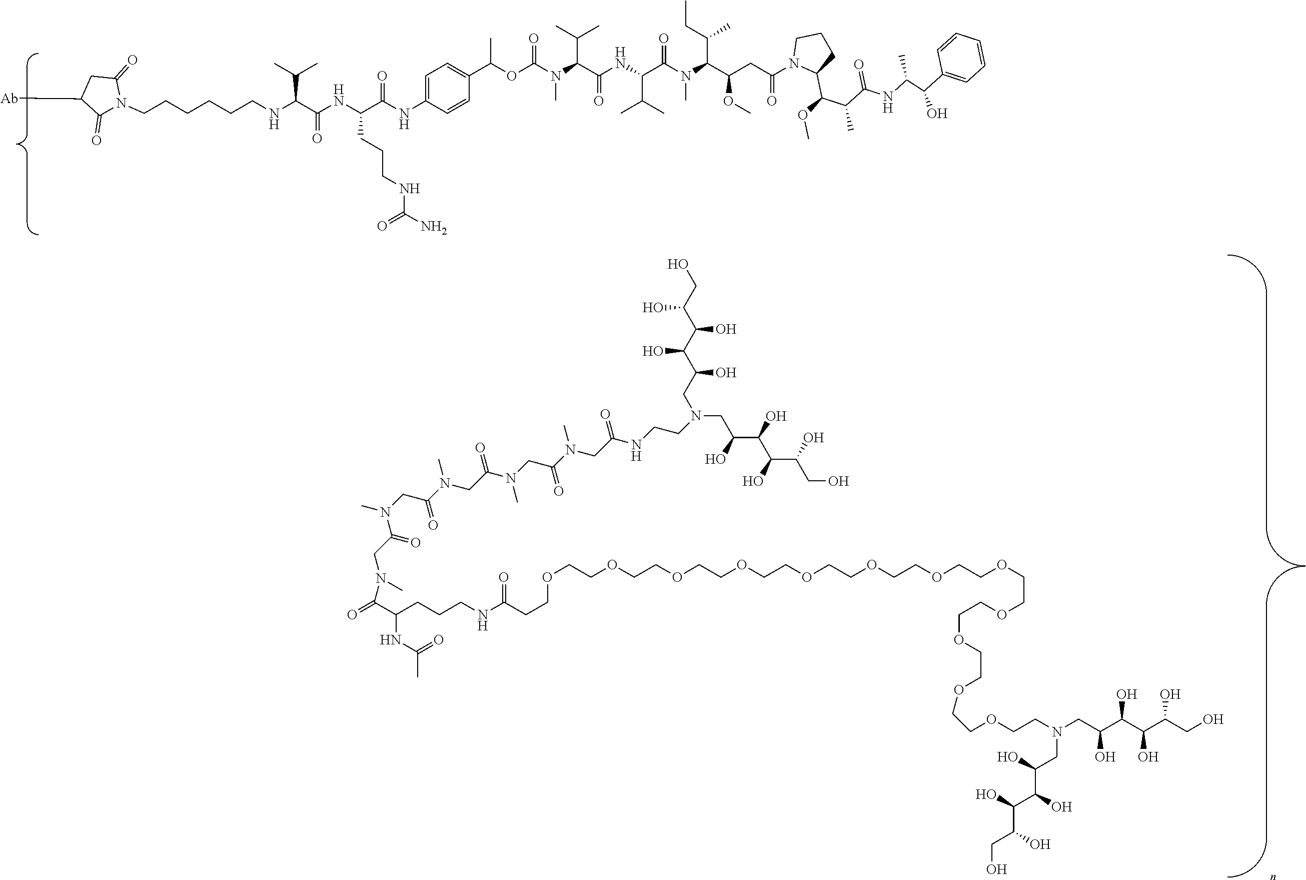 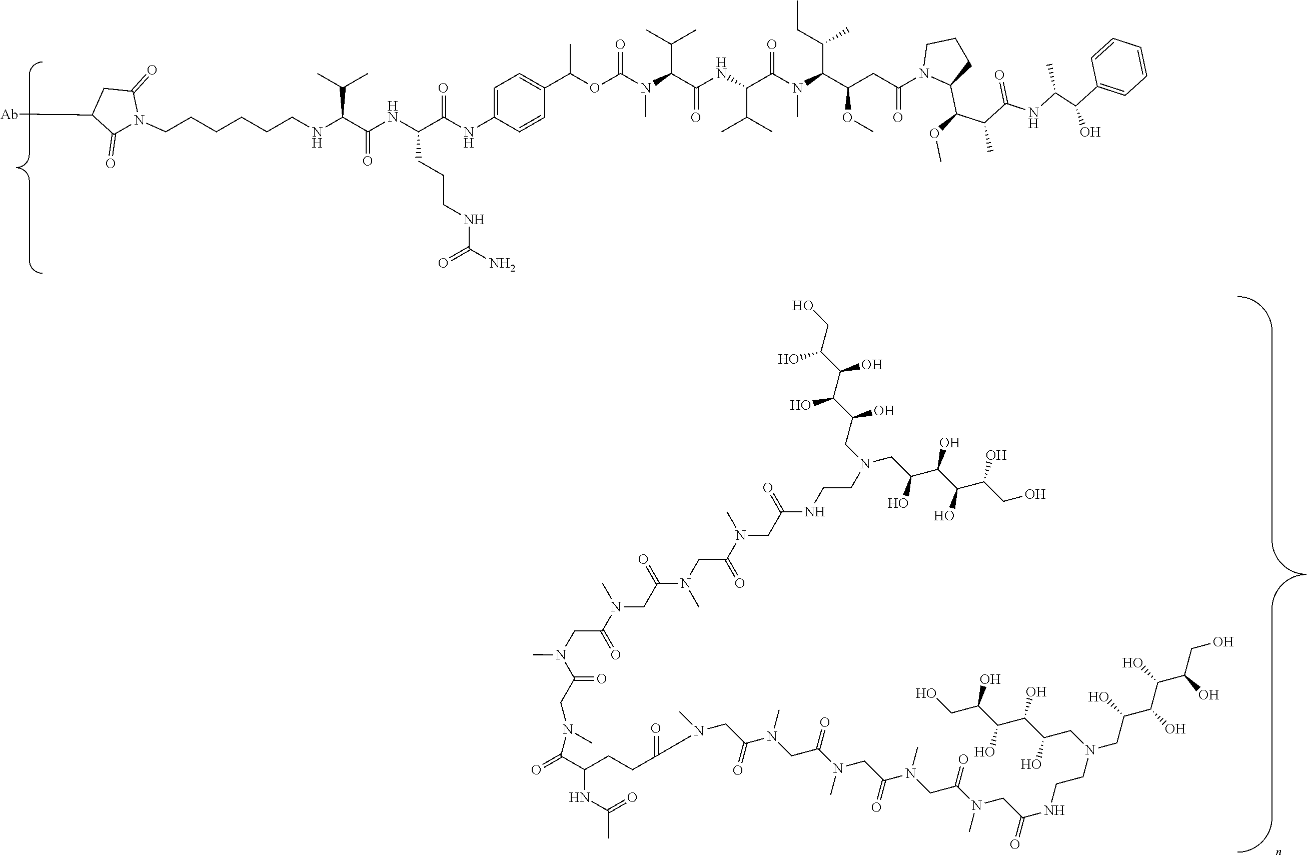 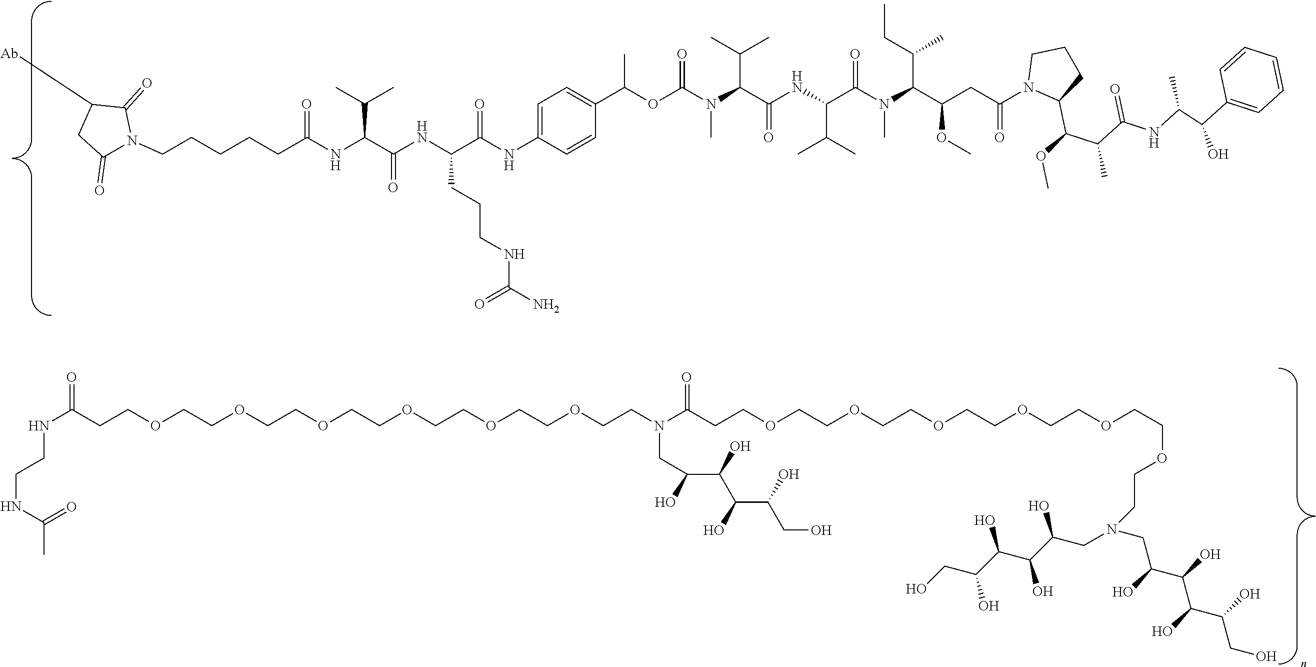 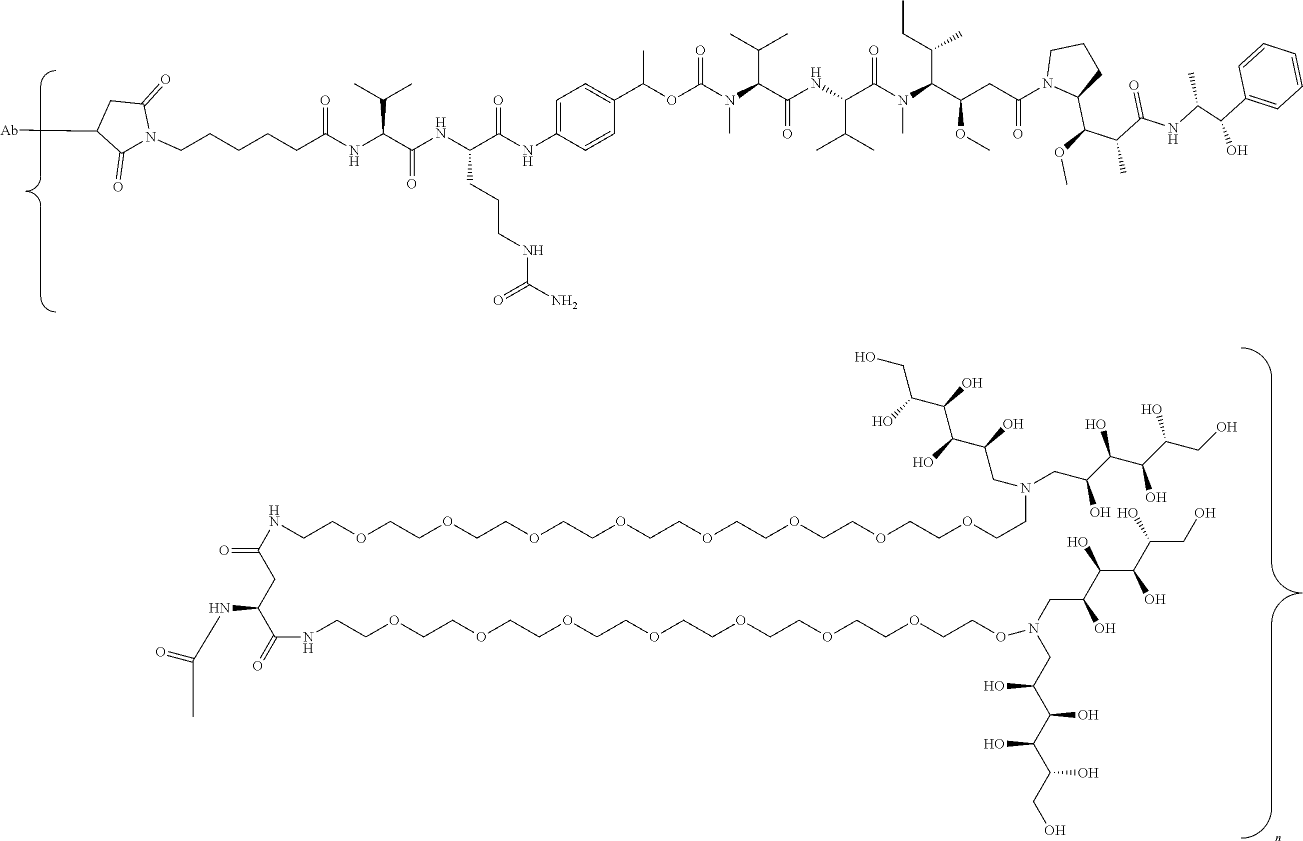 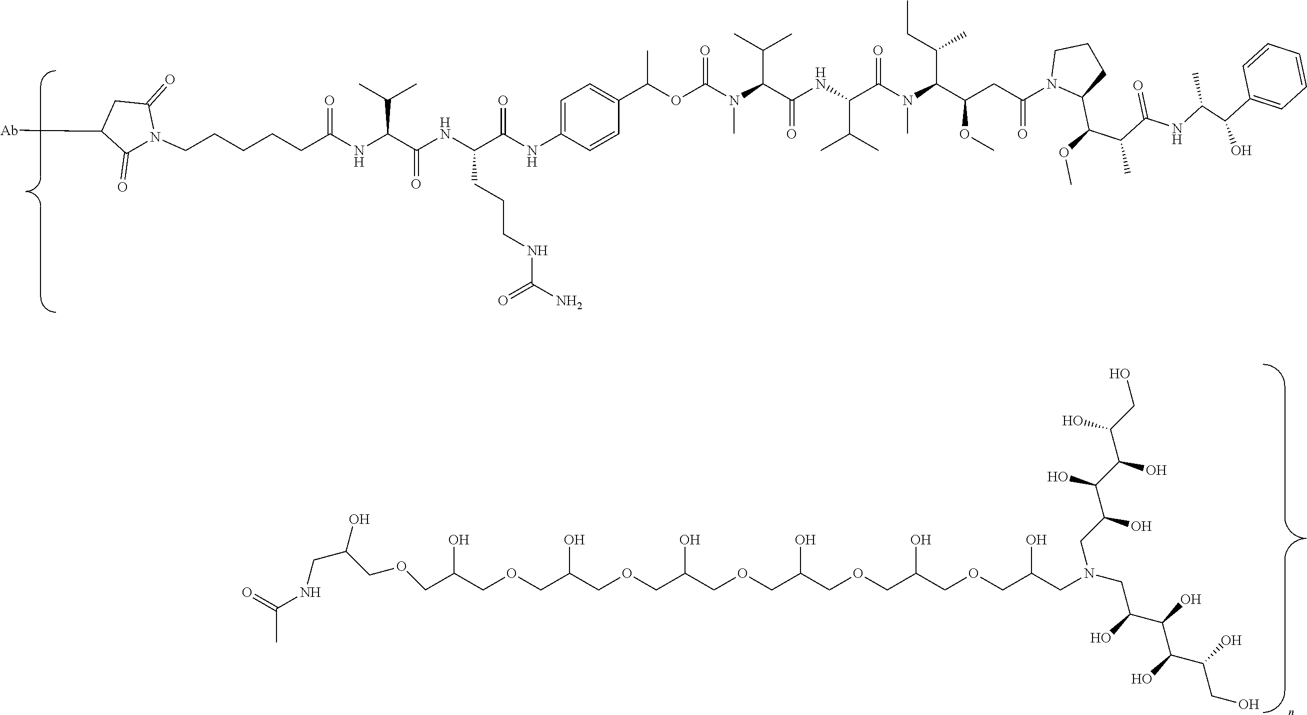 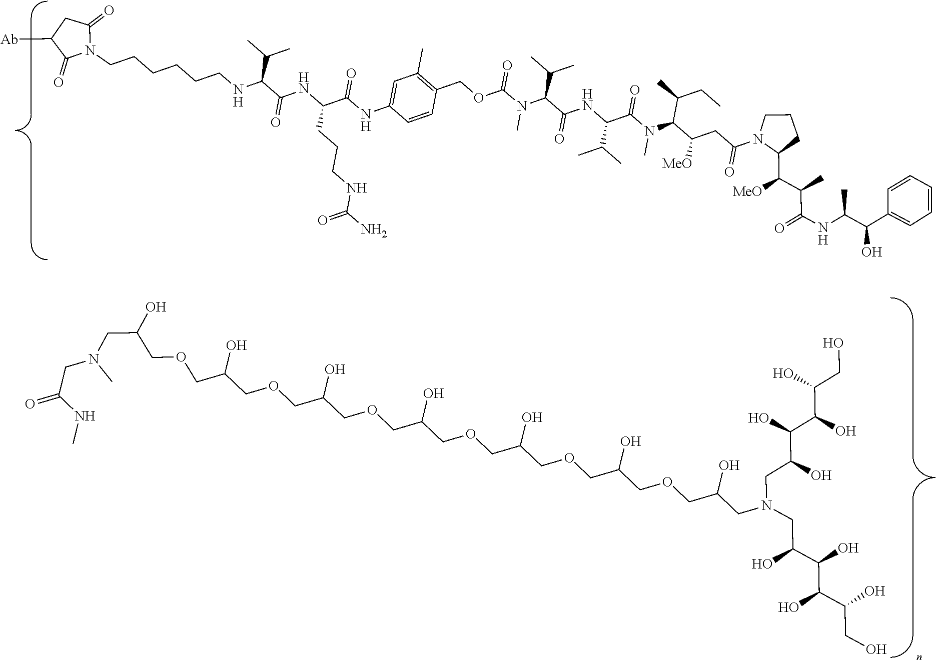 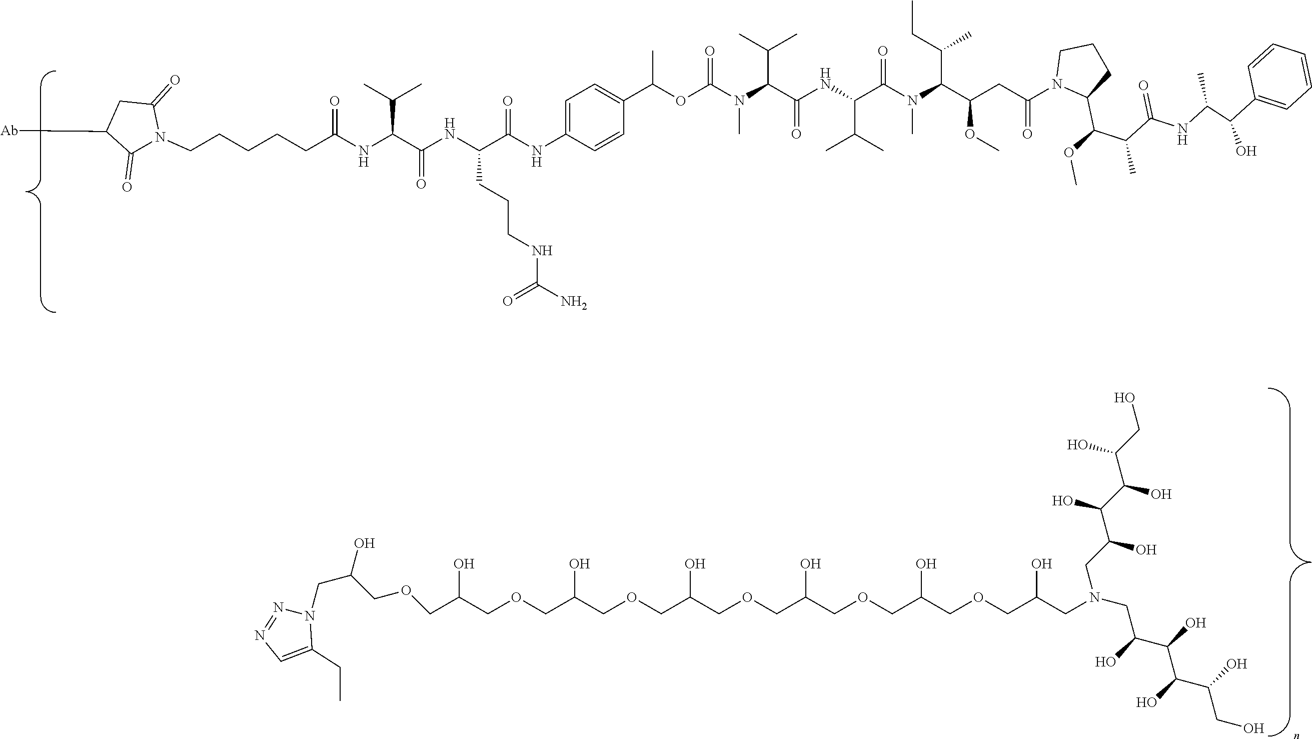 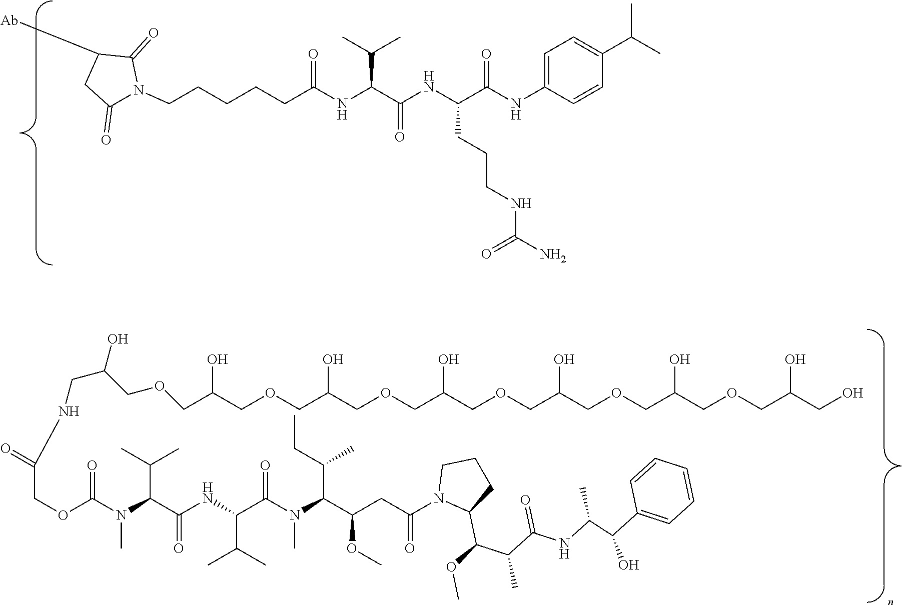 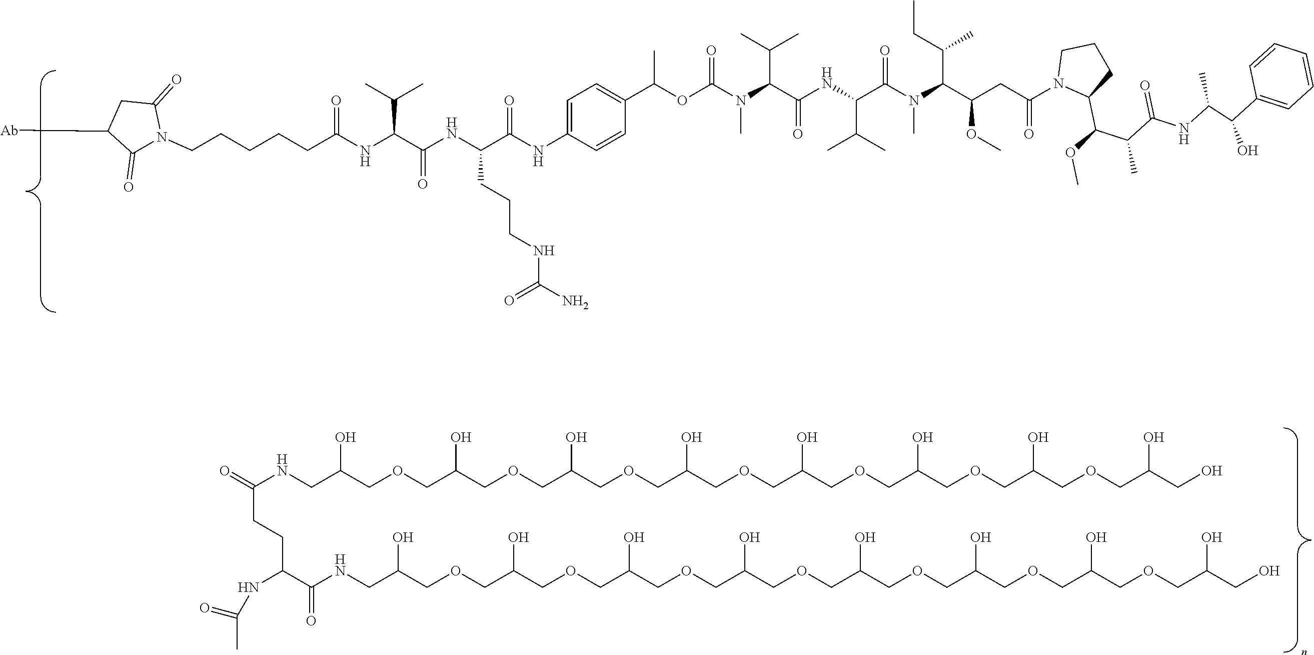 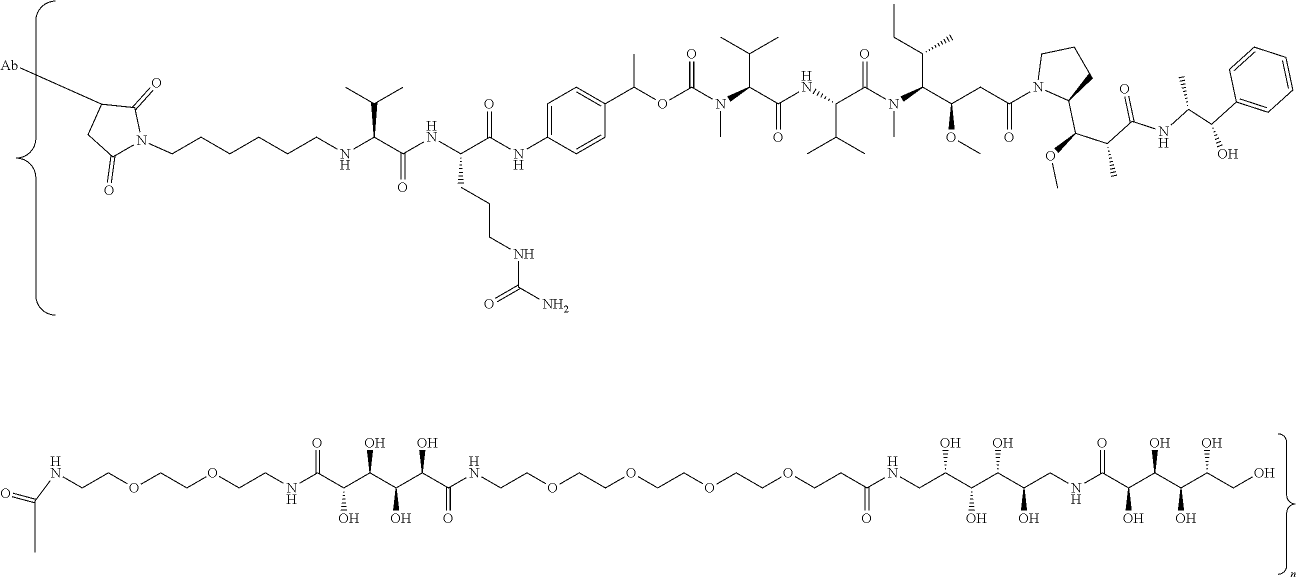 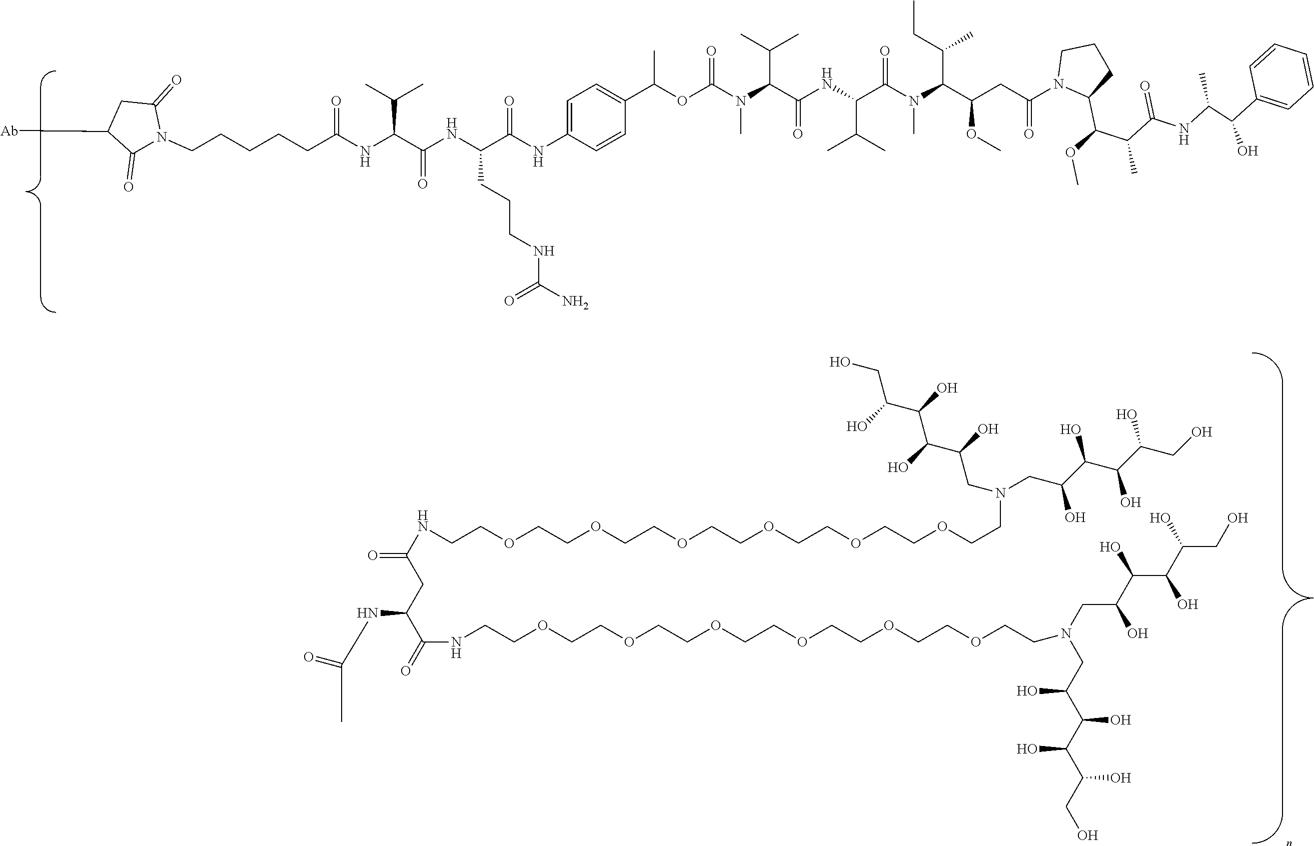 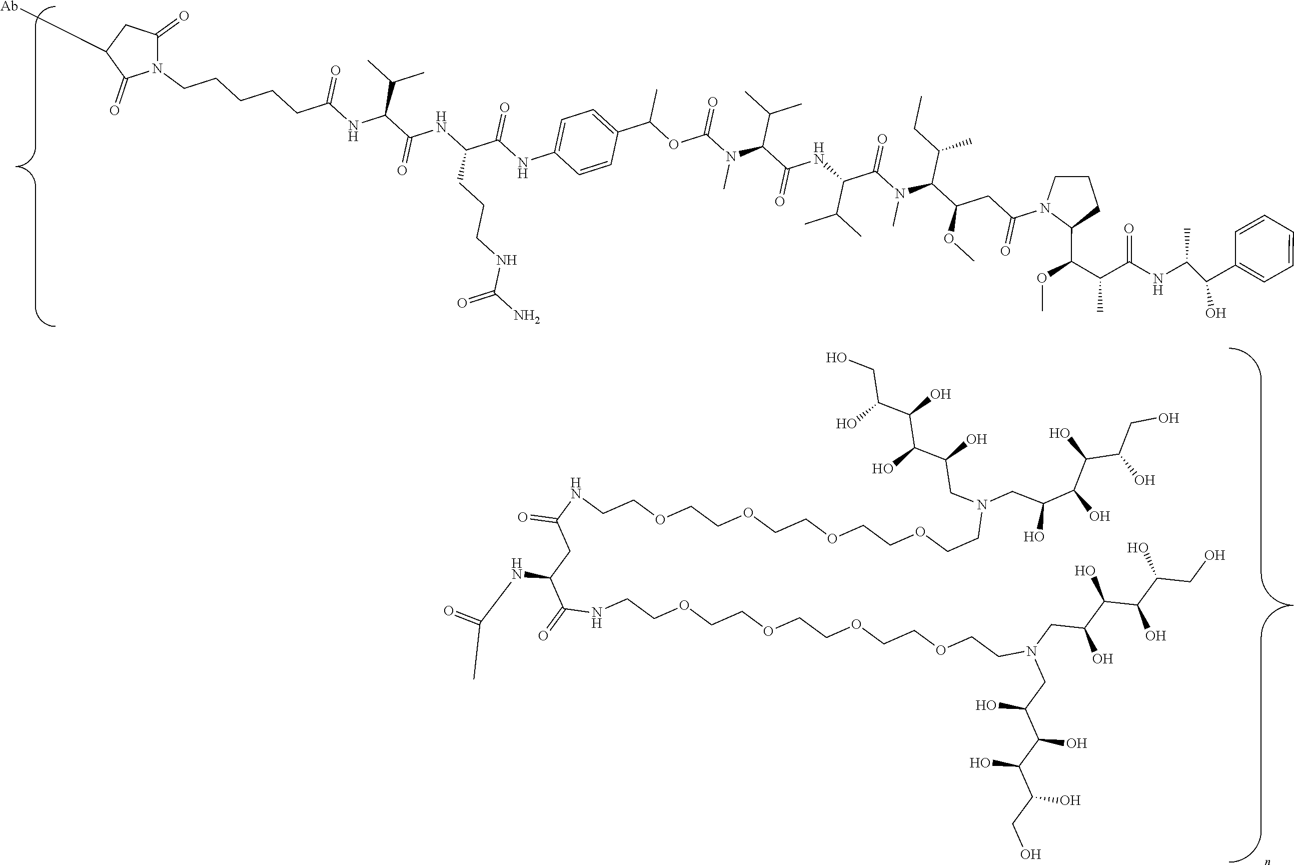 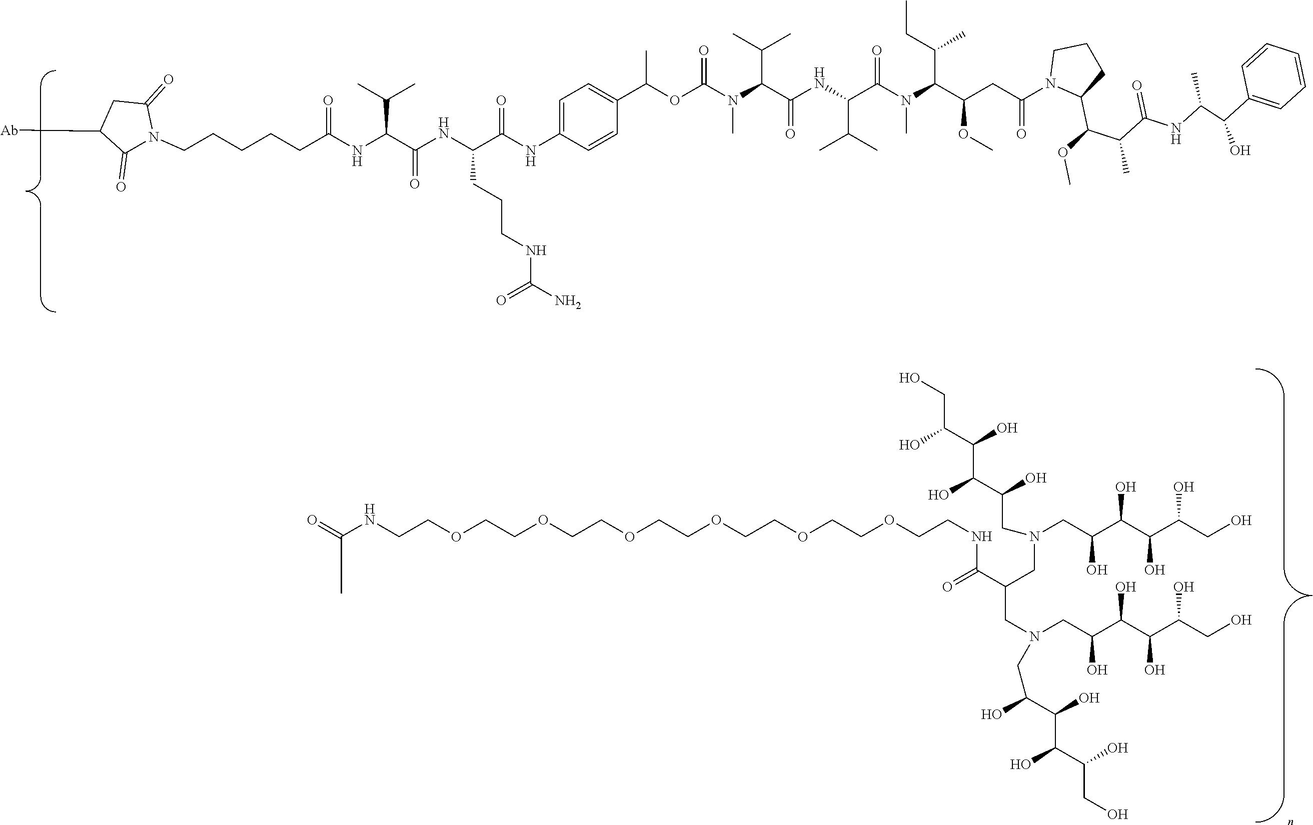 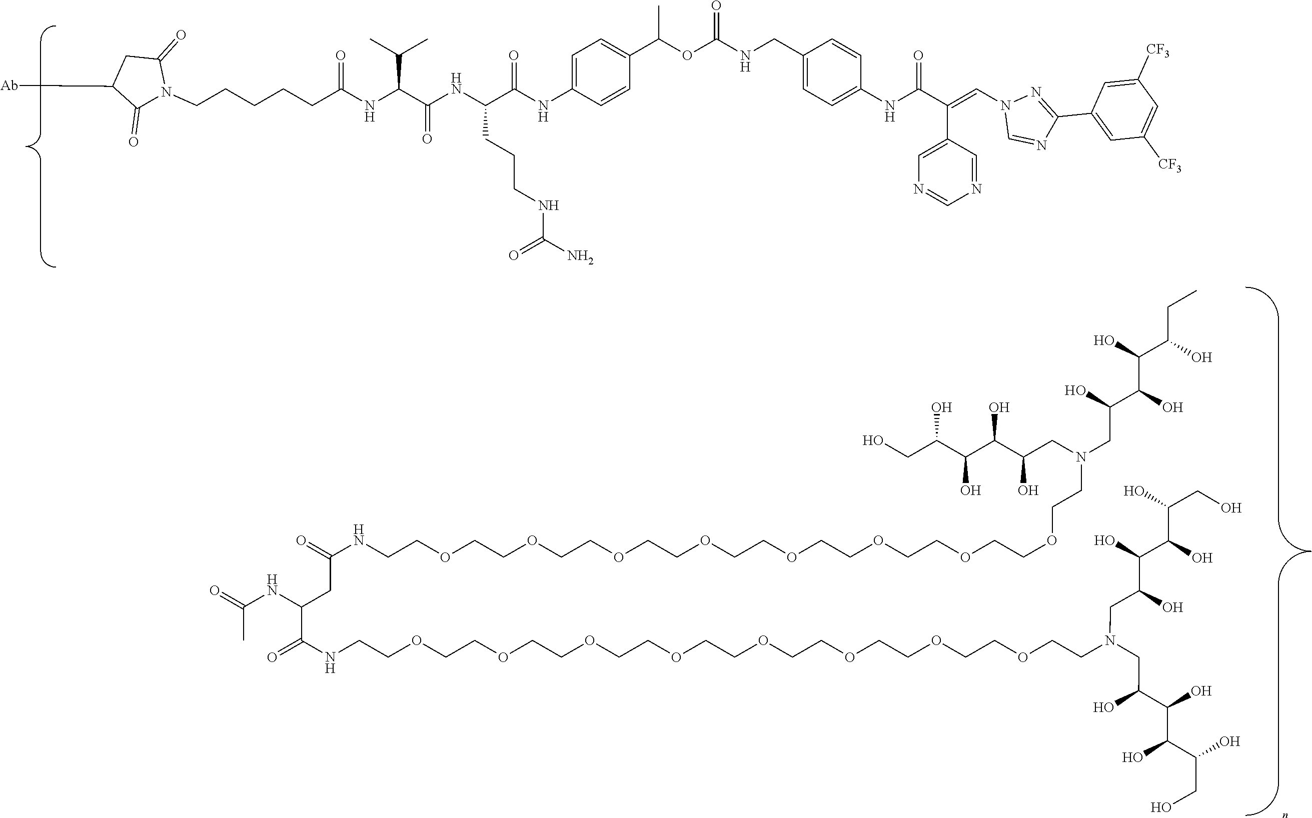 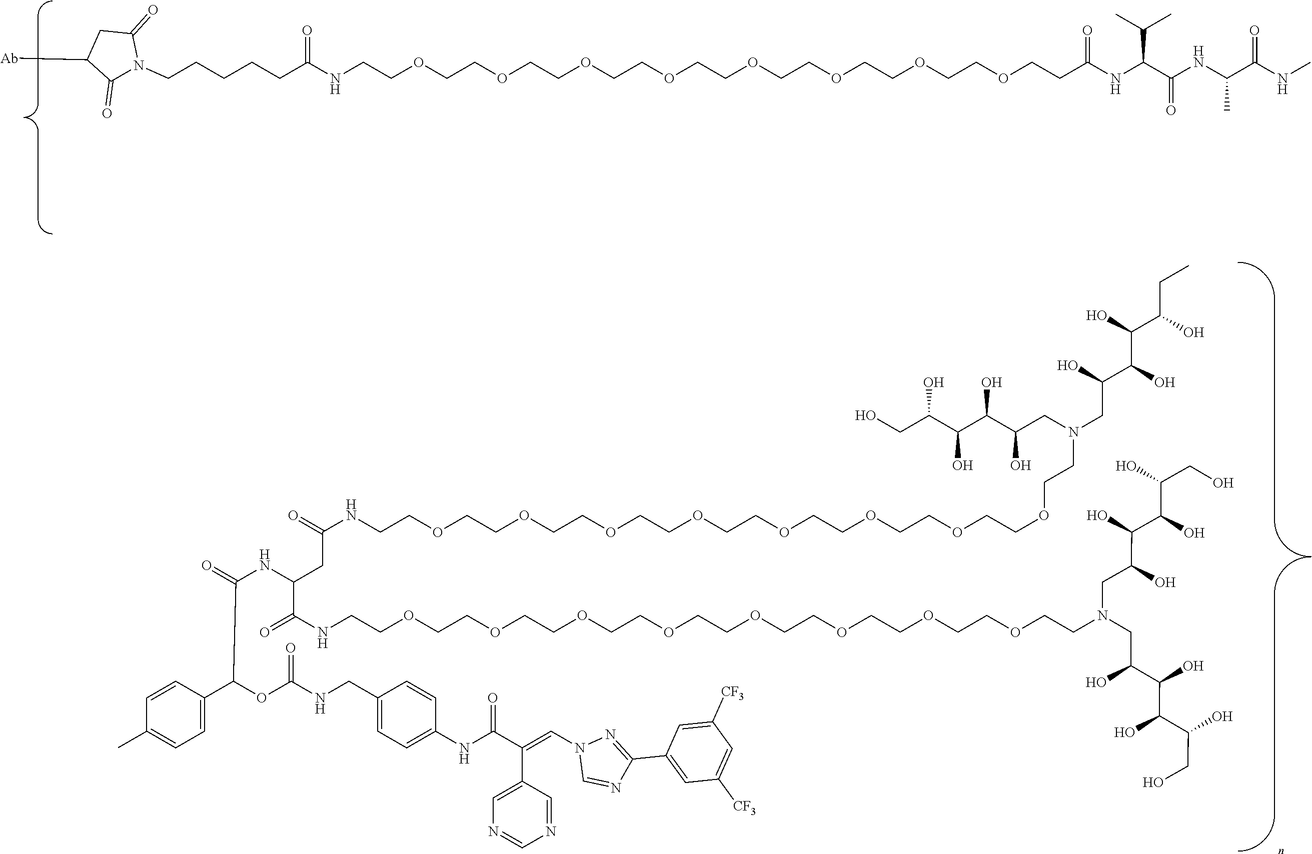 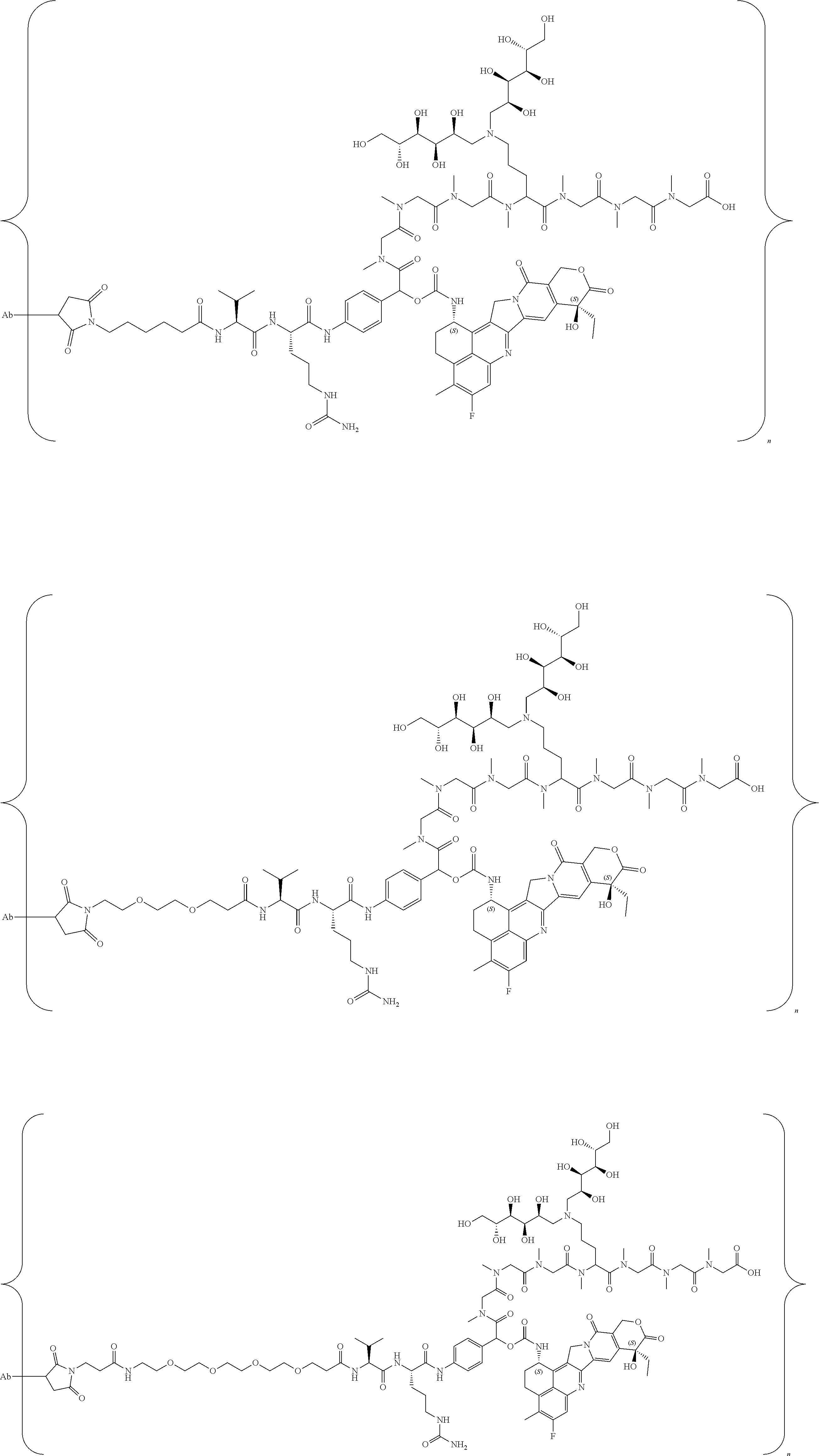 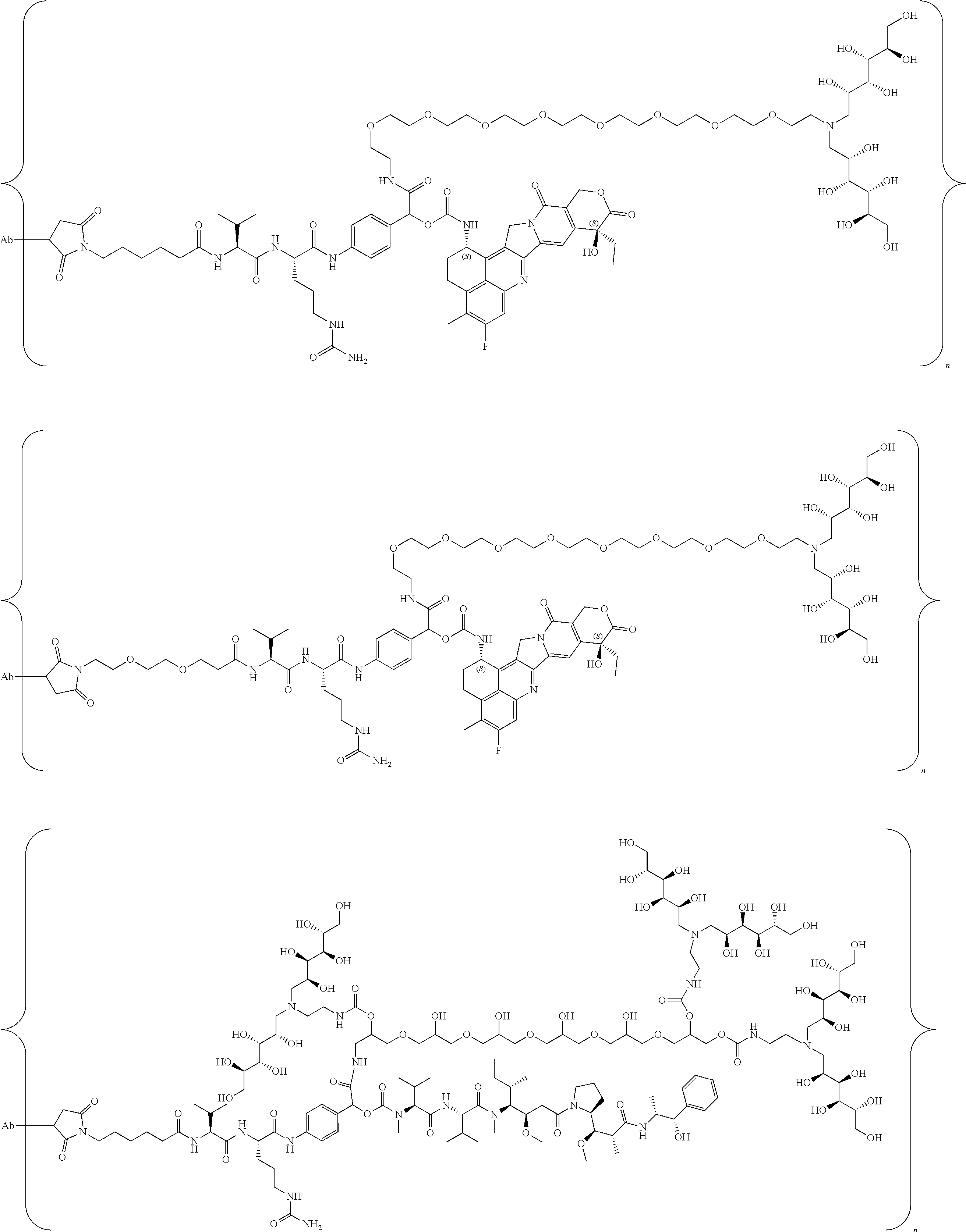 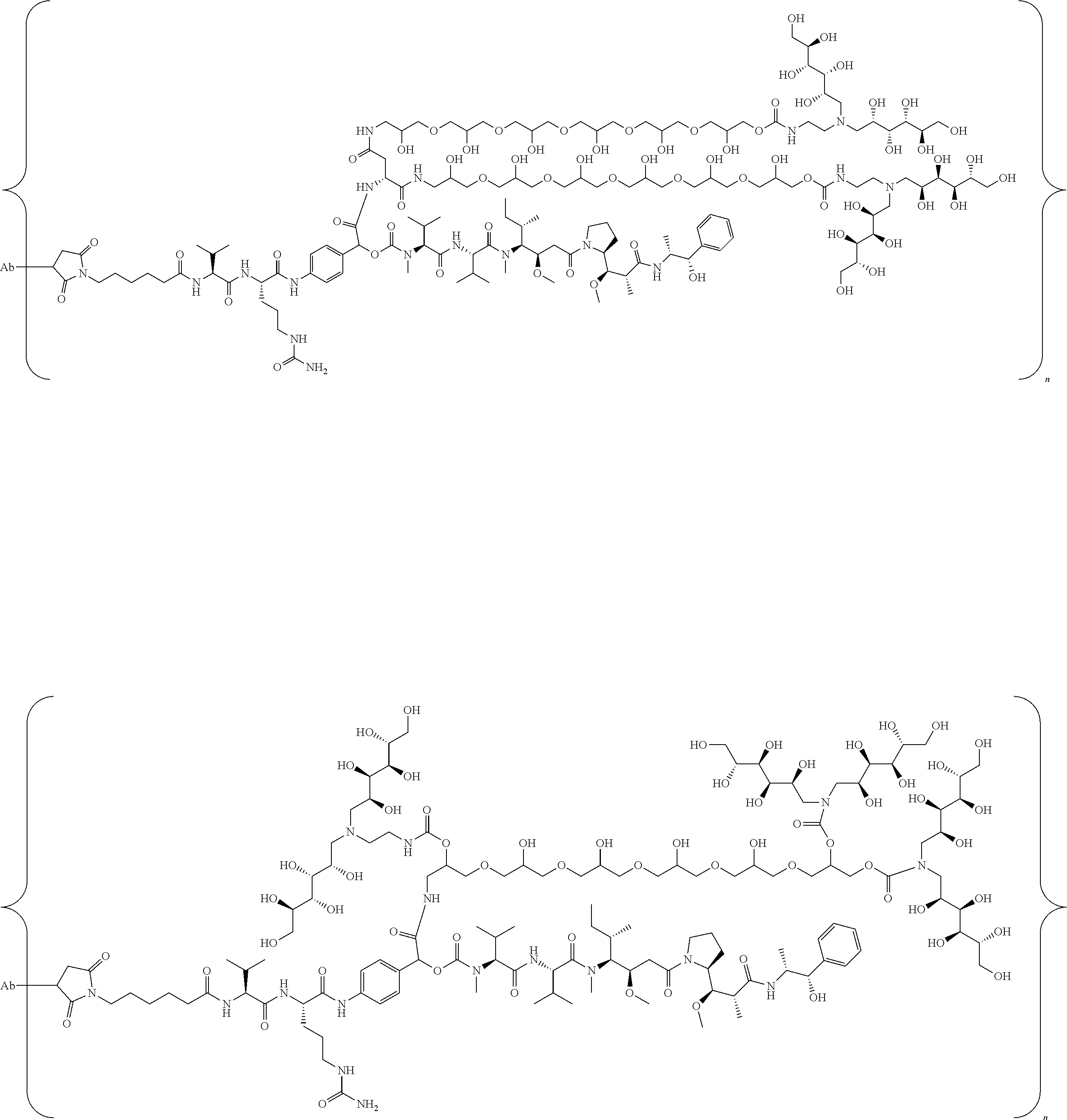 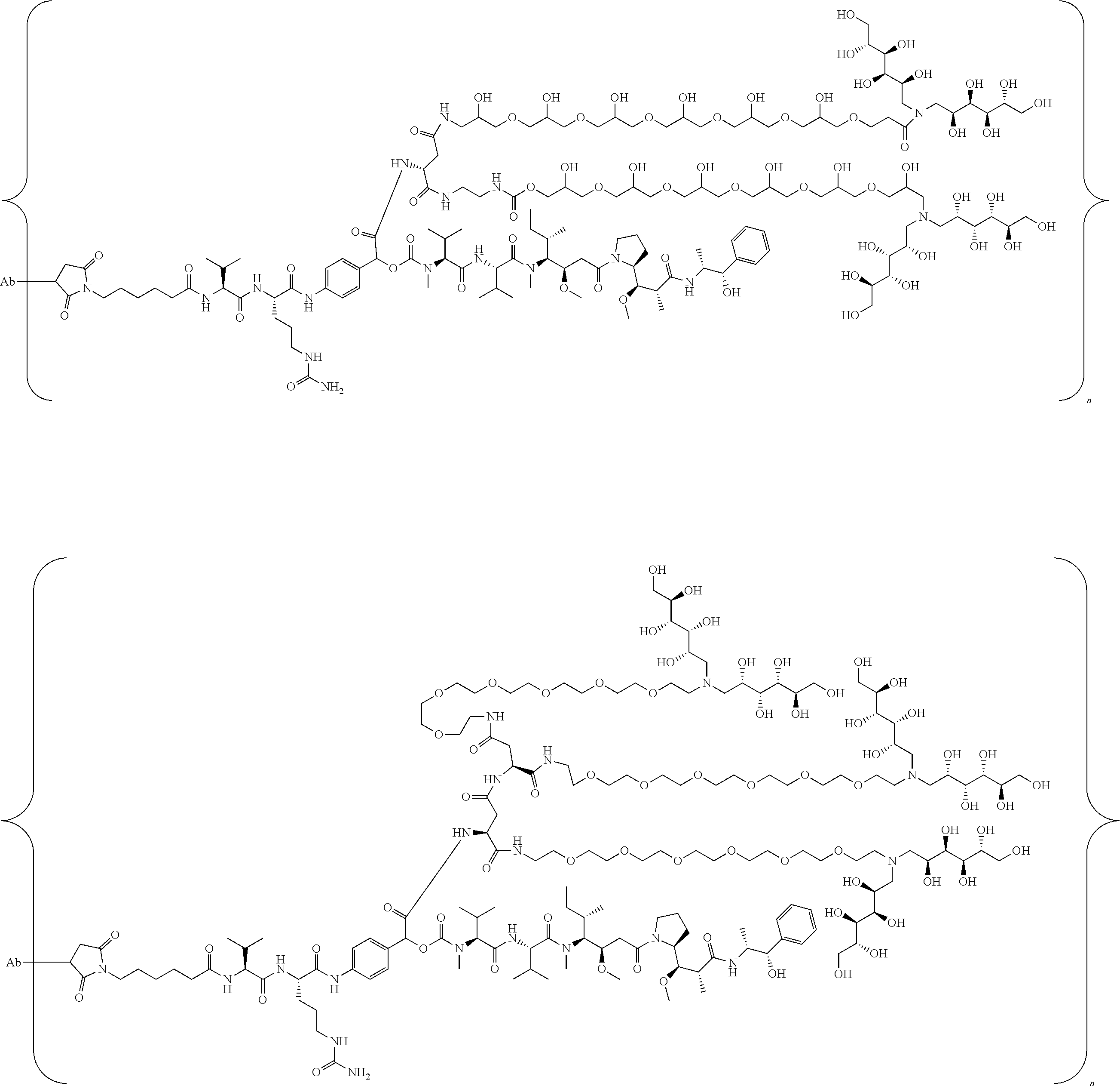 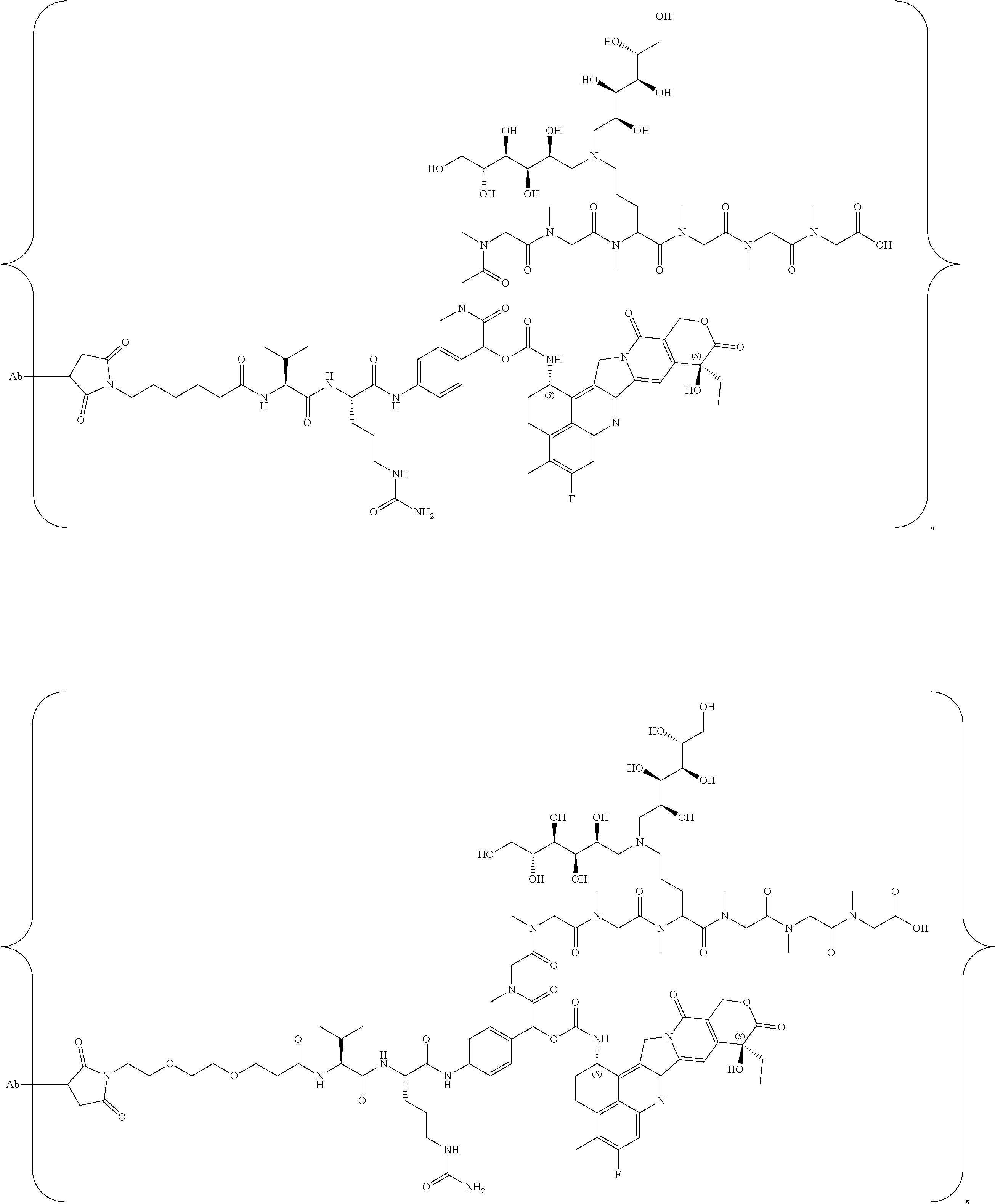 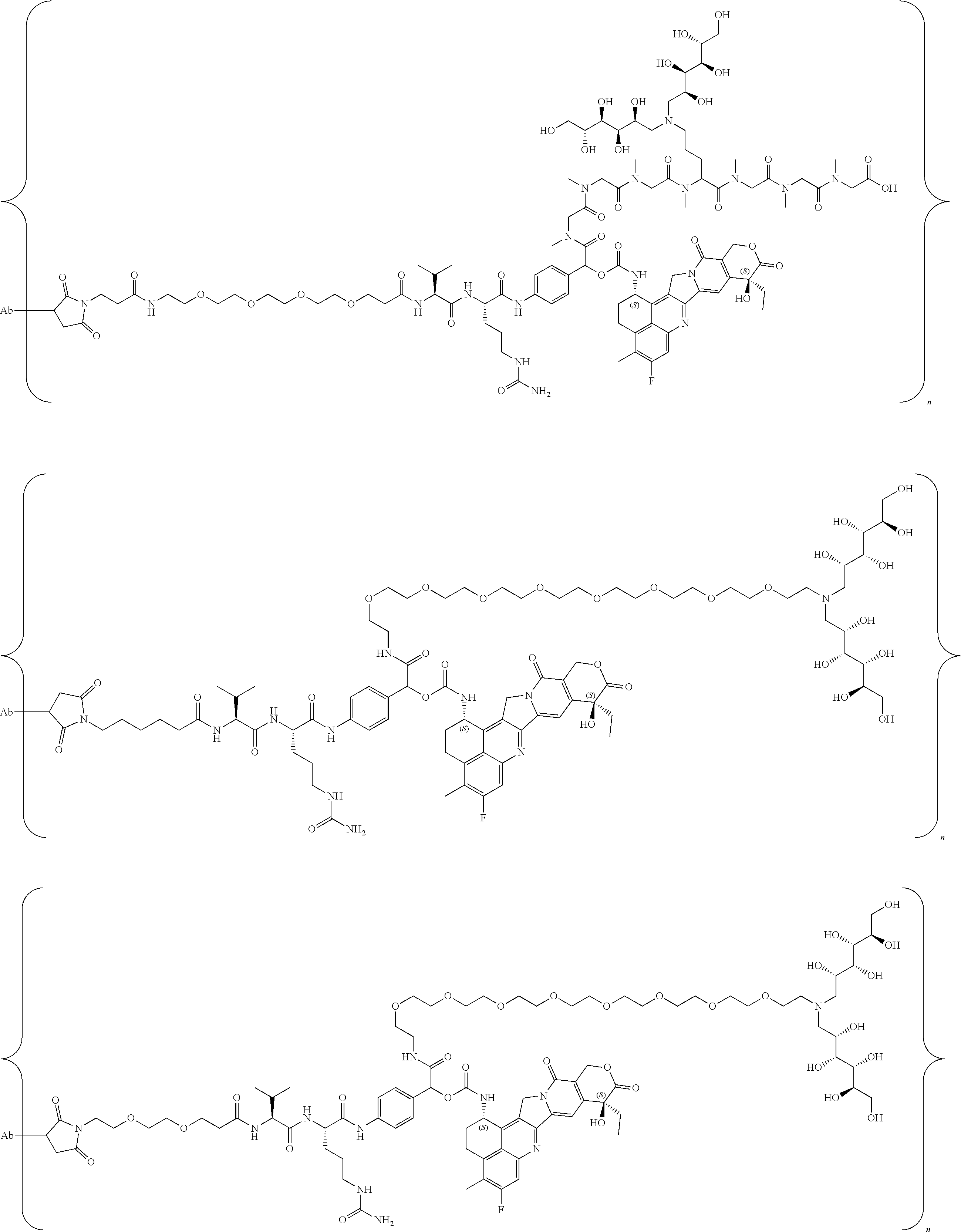 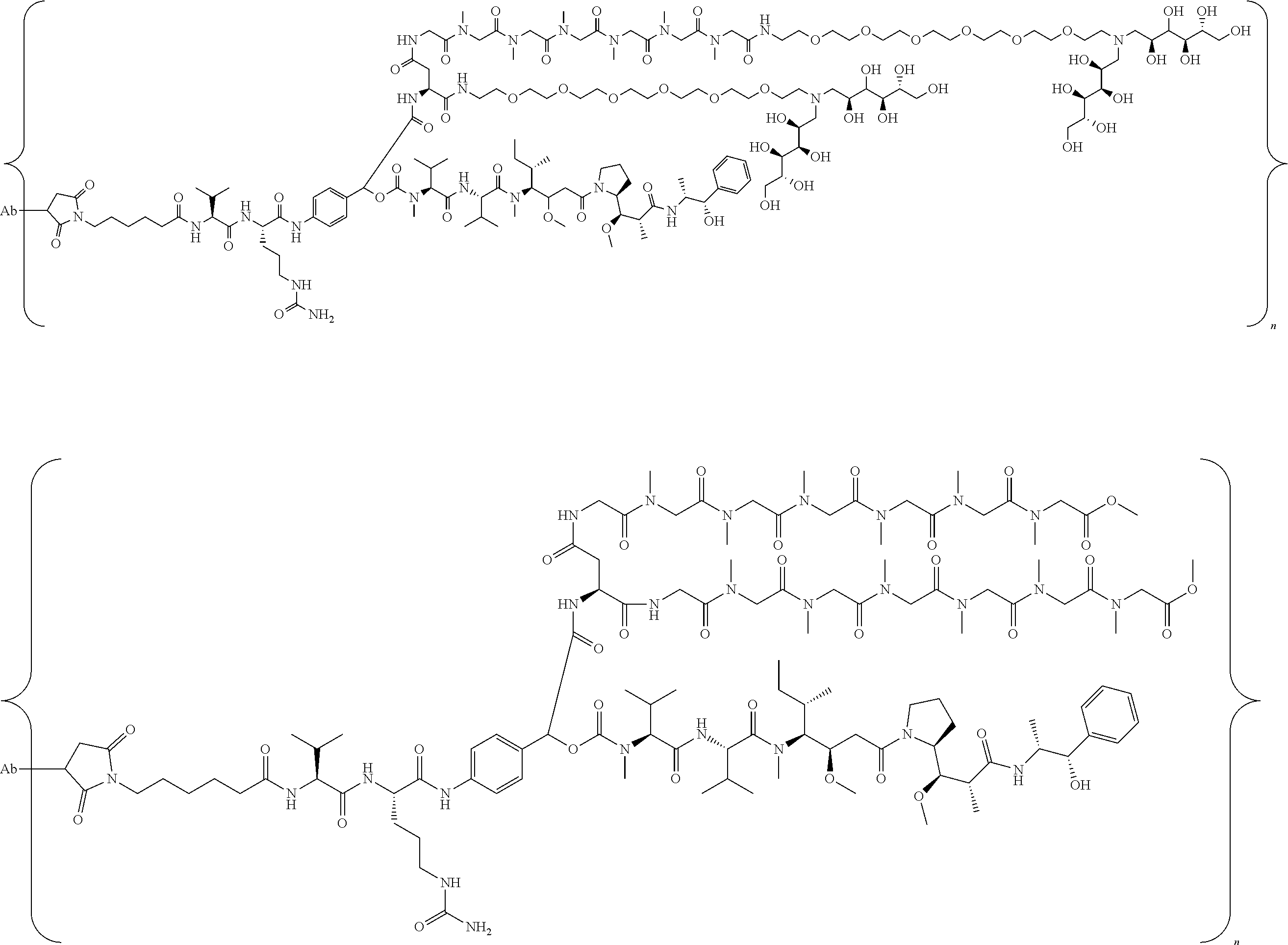 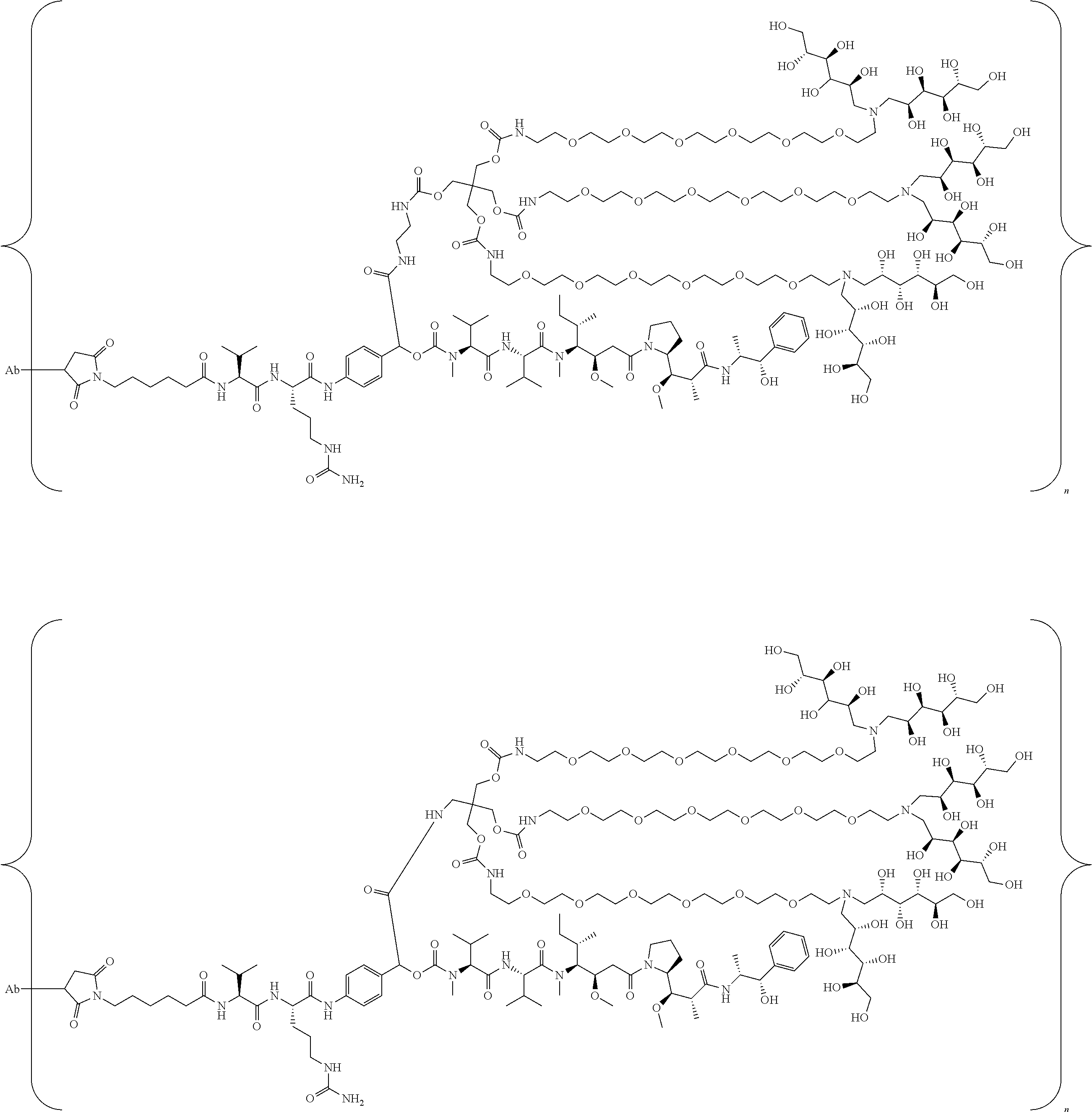 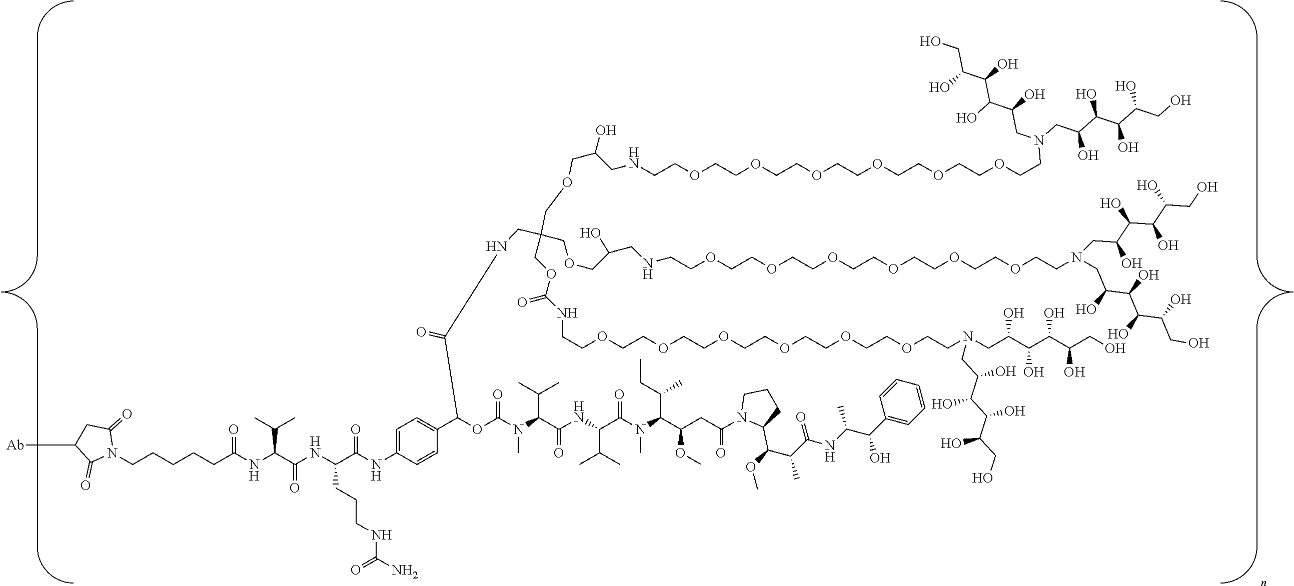 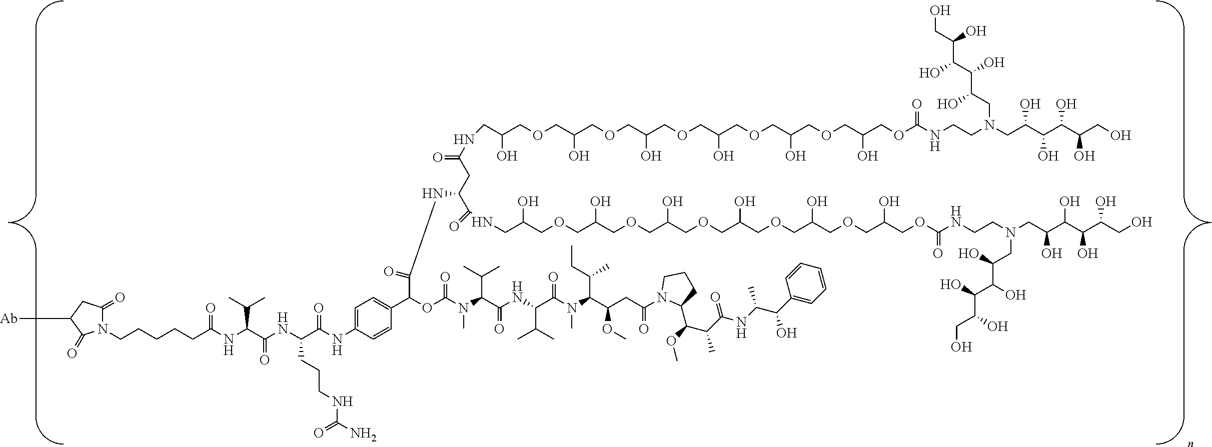 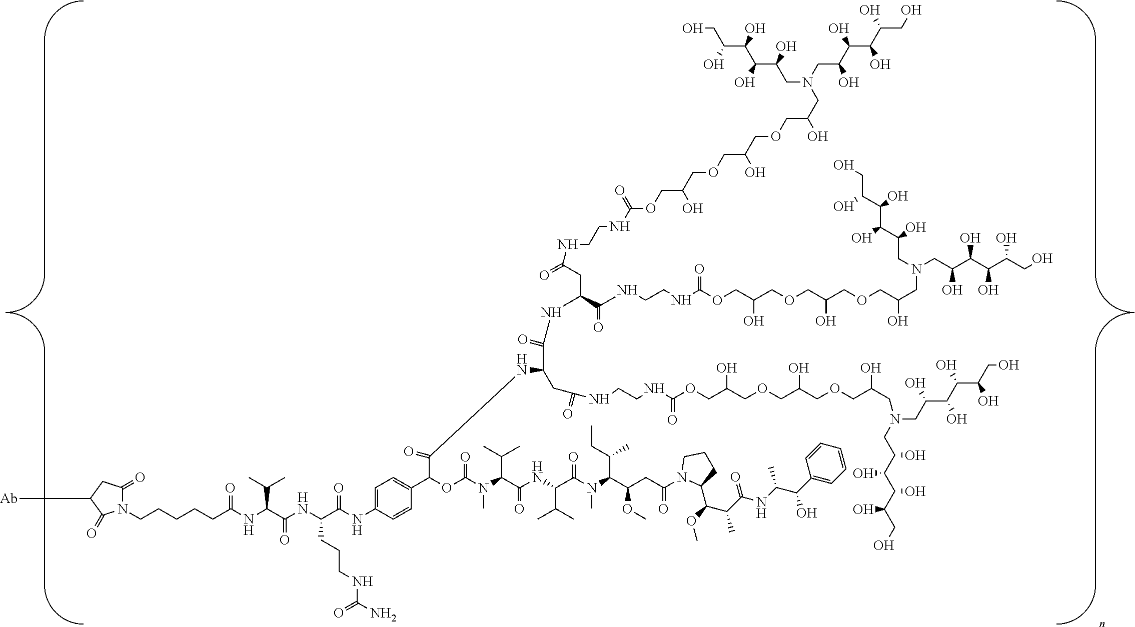 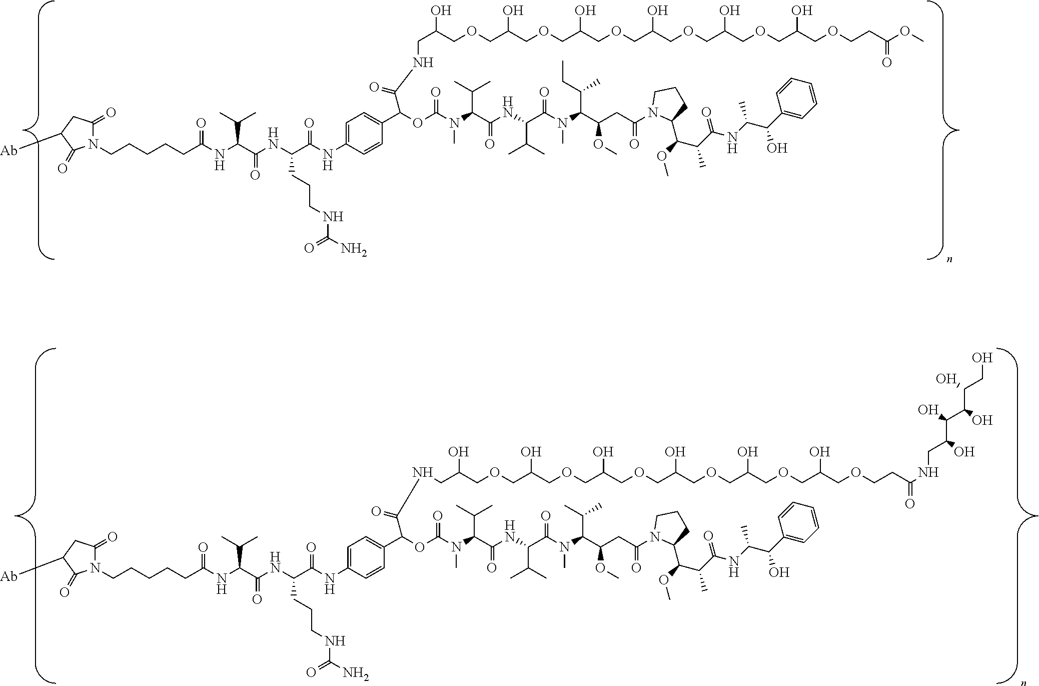 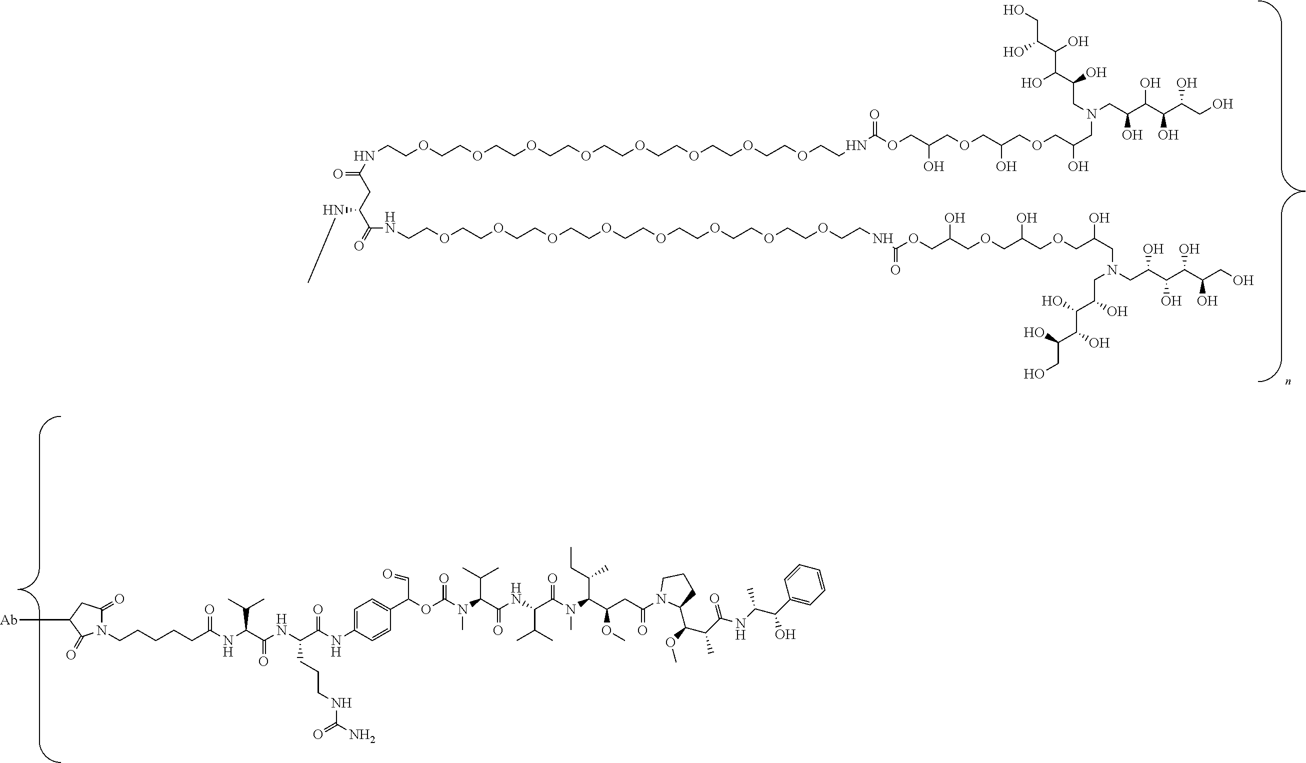 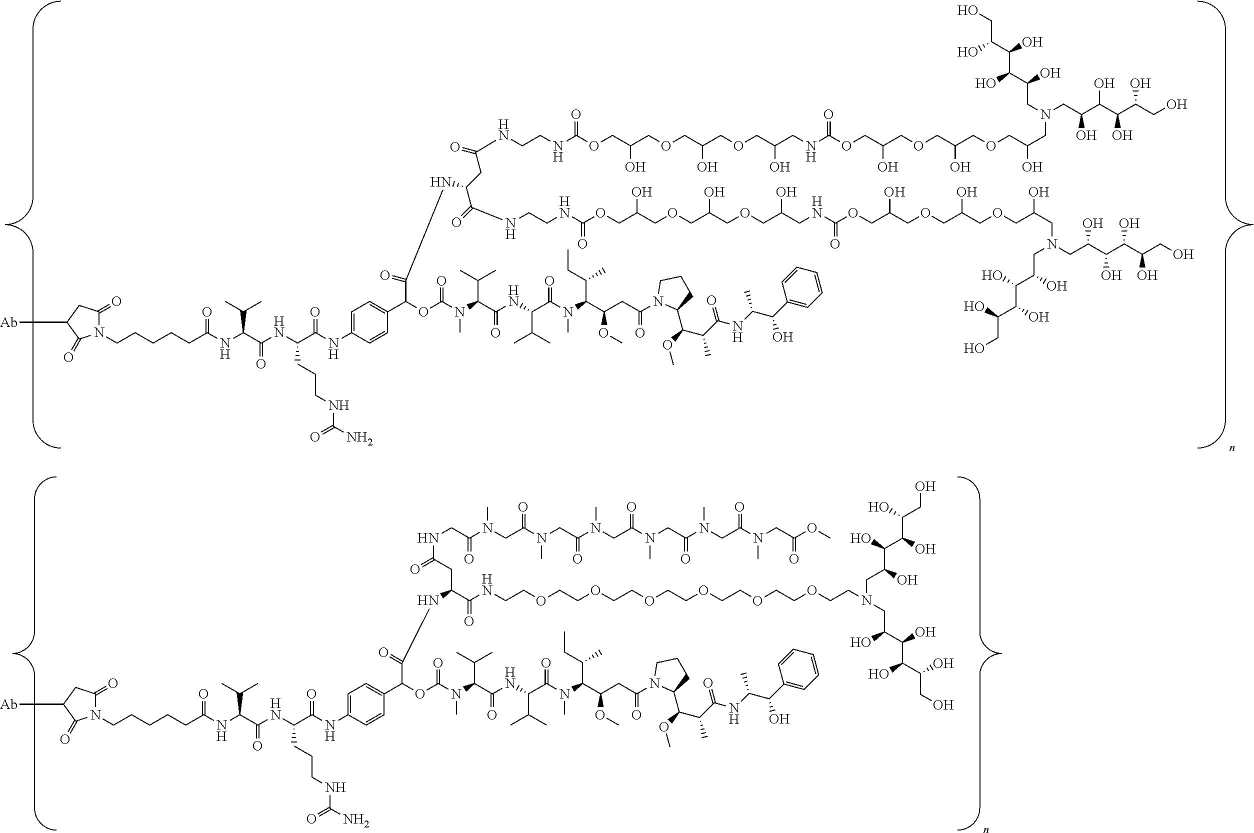 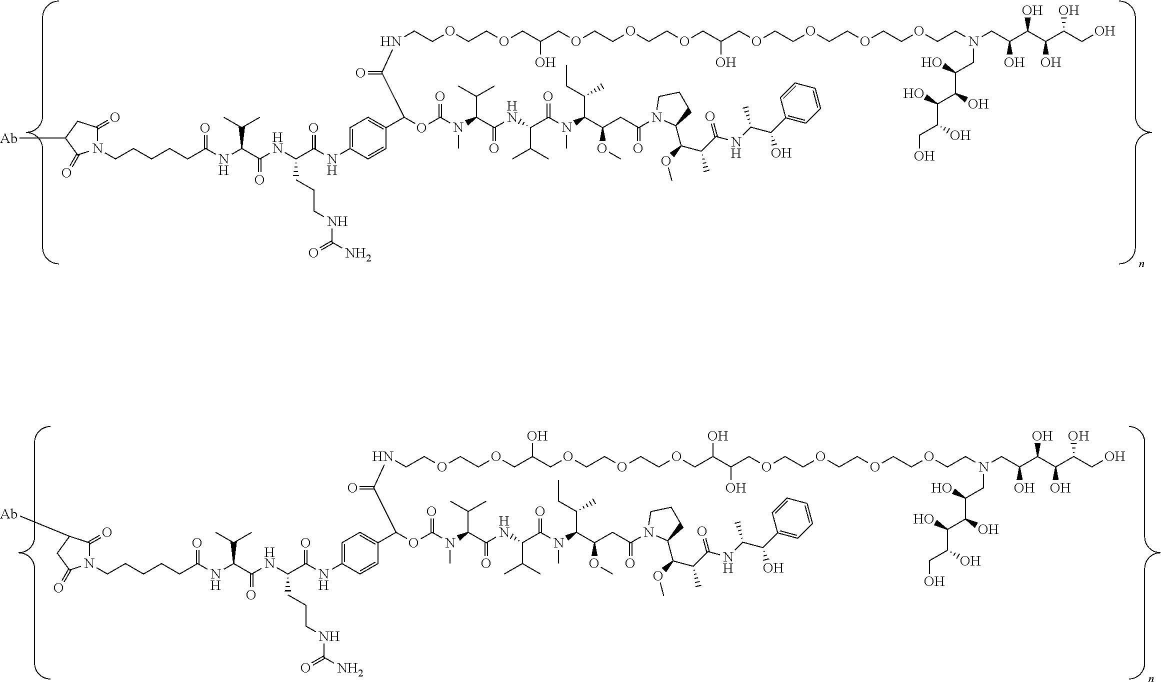 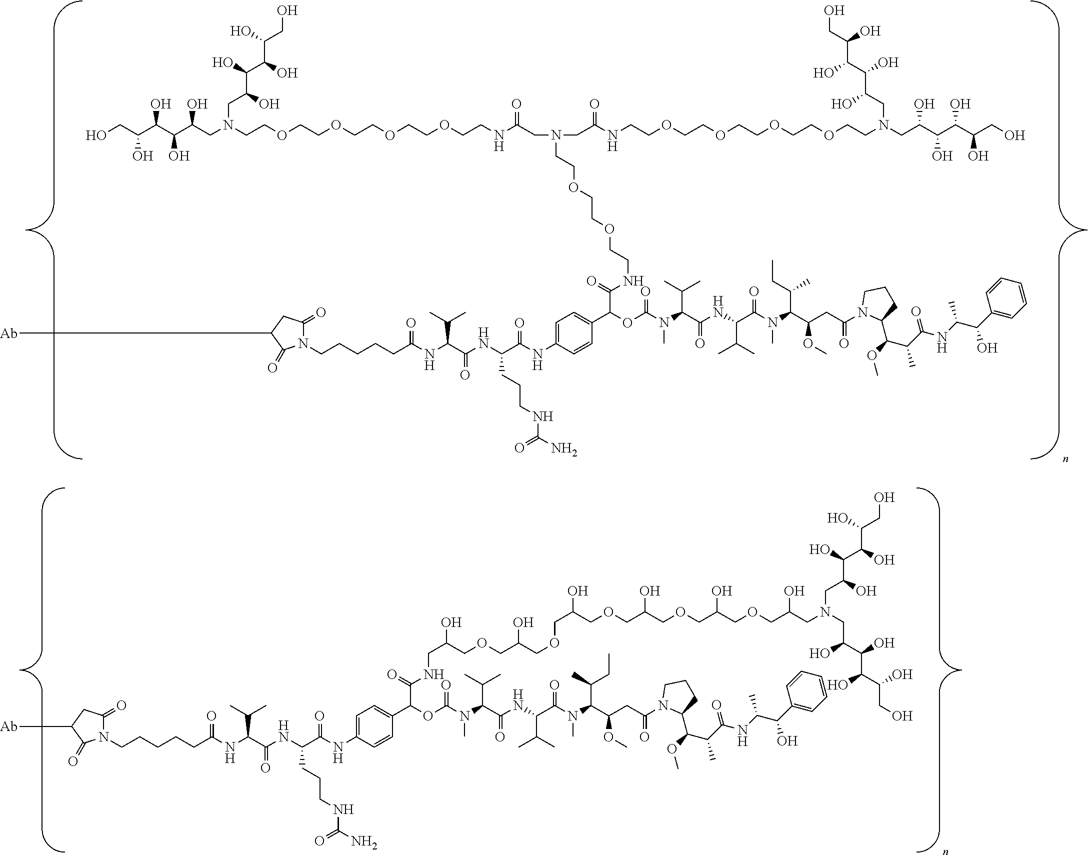 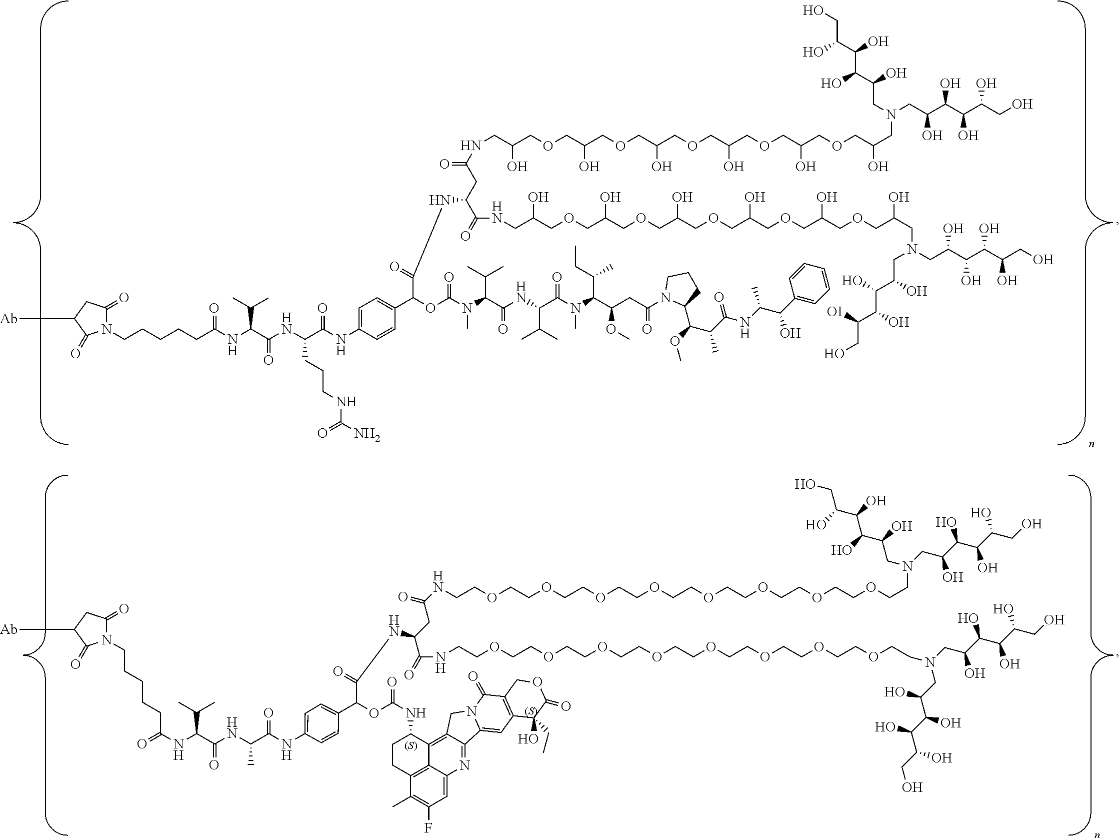 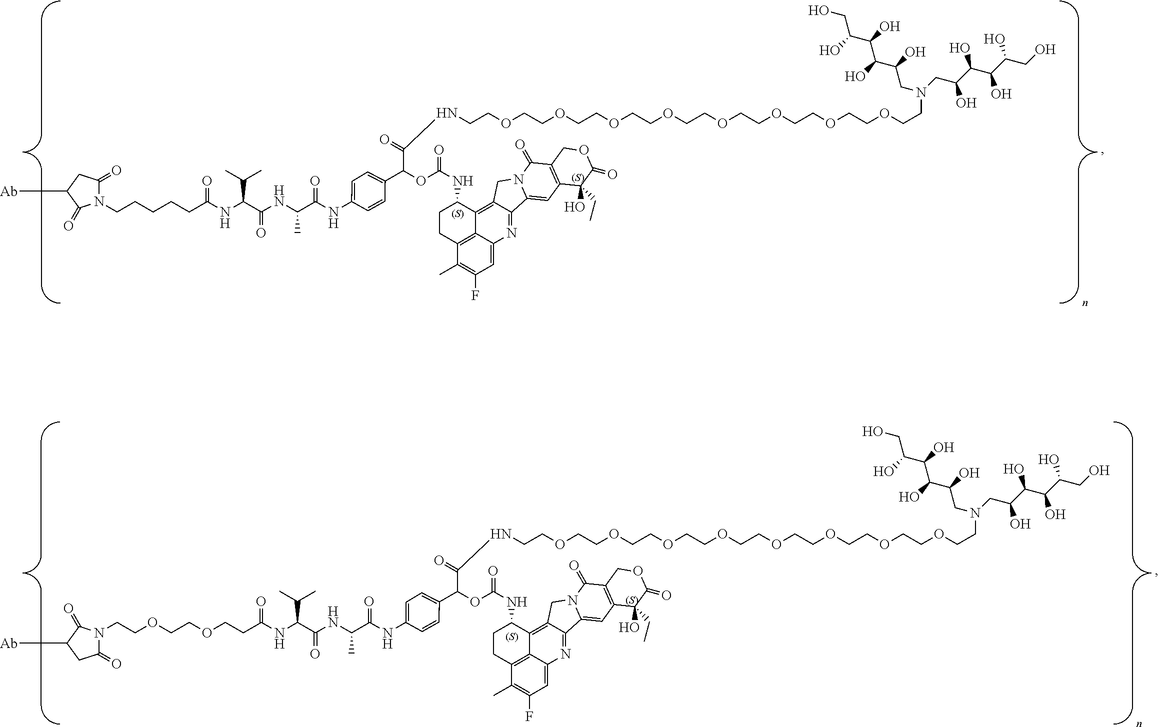 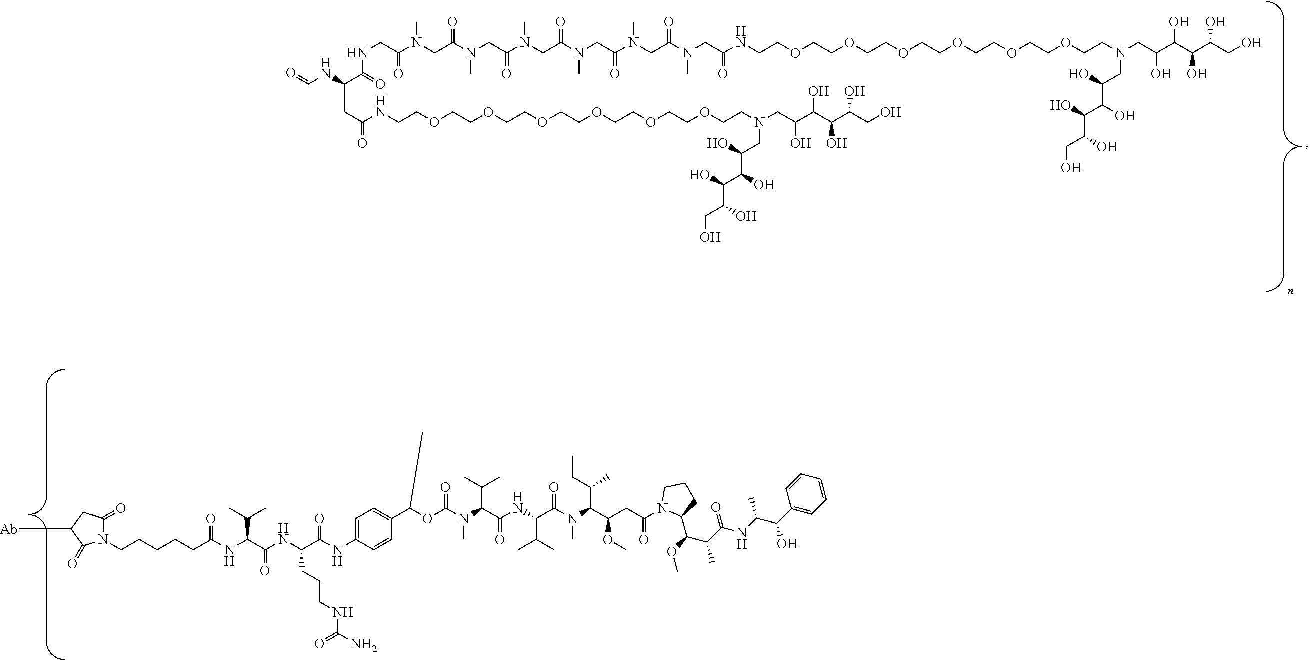 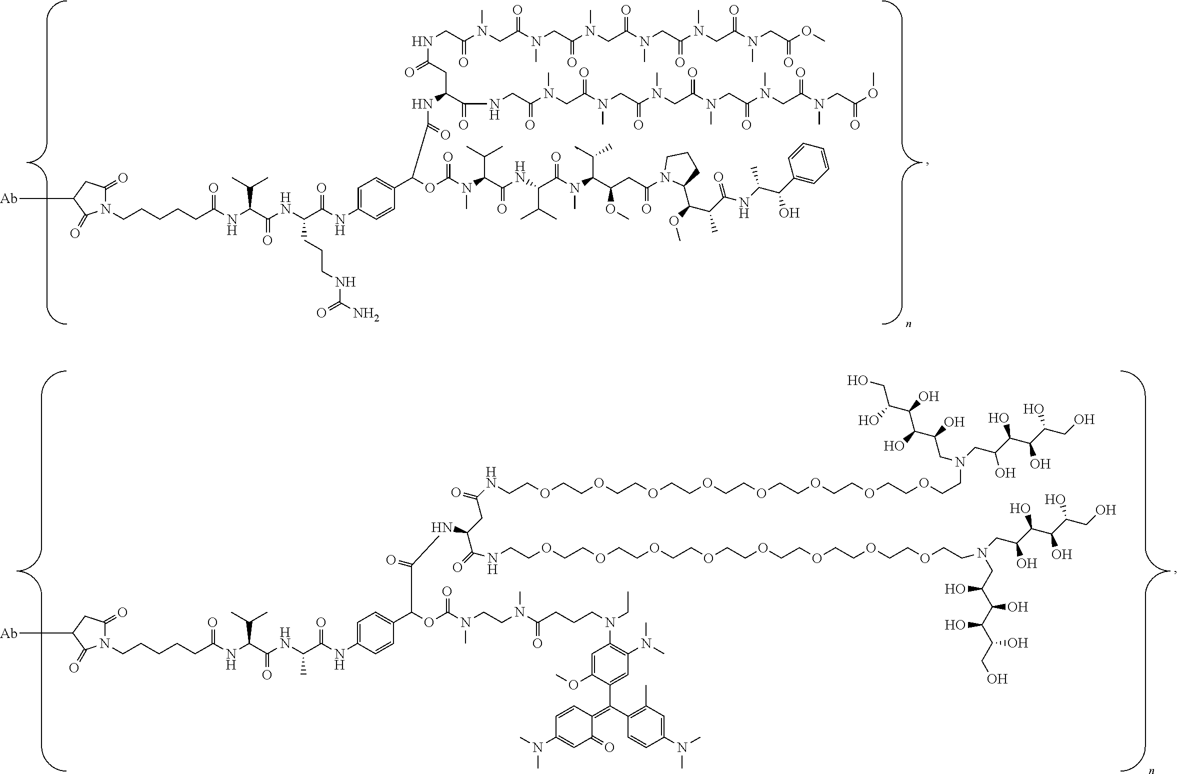 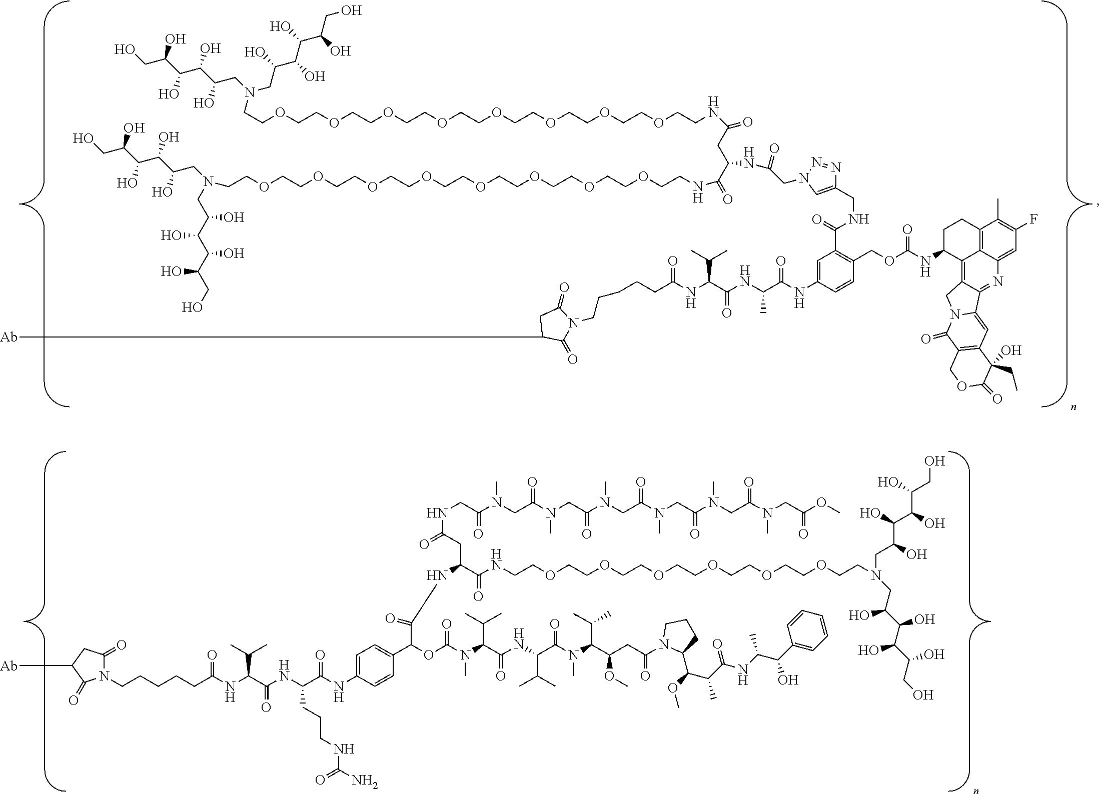
wherein Ab is a Targeting group and n is p^{load}.

111. The conjugate of any one of claims 102-110, wherein the target molecule is CD19, CD20, CD30, CD33, CD70, LIV-1, EGFRv3, or HER2.

112. The conjugate of any one of claims 102-111, wherein the target molecule is a cancer associated antigen.

113. The conjugate of any one of claims 102-111, wherein the target molecule is CD19, CD20, CD30, CD33, CD38, CA125, HER2, MUC-1, prostate-specific membrane antigen (PSMA), CD44 surface adhesion molecule, mesothelin (MLSN), carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-A1, IL-13R-a2, GD2, 1p19q, HER2, ABL1, AKT1, ALK, APC, AR, ATM, BRAF, BRCA1, BRCA2, cKIT, cMET, CSF1R, CTNNB1, FGFR1, FGFR2, FLT3, GNA11, GNAQ, GNAS, HRAS, IDH1, IDH2, JAK2, KDR (VEGFR2), KRAS, MGMT, MGMT-Me, MLH1, MPL, NOTCHI, NRAS, PDGFRA, Pgp, PIK3CA, PR, PTEN, RET, RRM1, SMO, SPARC, TLE3, TOP2A, TOPO1, TP53, TS, TUBB3, VHL, CDH1, ERBB4, FBXW7, HNF1A, JAK3, NPM1, PTPN11, RB1, SMAD4, SMARCB1, STK1, MLH1, MSH2, MSH6, PMS2, ROS1, ERCC1, 5T4 (TPBG), B7-H3, CCR7, CD105, CD22, CD46, CD47, CD56, CD70, CD71, CD79b, CDH6, CLDN6, CLDN18.2, CLEC12A, DLL3, DR5, ERBB3 (HER3), EPCAM, FOLR1, IGF1R, IL2RA (CD25), IL3RA, ITGB6, LIV-1, LRRC15, mesothelin (MSLN), NaPi2b (SLC34A2), nectin-4, PTK7, ROR1, SEZ6, SLC44A4, SLITRK6, Tissue Factor (TF), TROP2, or B7-H4.

114. The conjugate of any one of claims 102-104, wherein the Targeting group is an antibody, or fragment thereof, comprising rituximab (Rituxan®), trastuzumab (Herceptin®), pertuzumab (Perjeta®)), bevacizumab (Avastin®), ranibizumab (Lucentis®), cetuximab (Erbitux®), alemtuzumab (Campath®), panitumumab (Vectibix®), ibritumomab tiuxetan (Zevalin®), tositumomab (Bexxar®), ipilimumab, zalutumumab, dalotuzumab, figitumumab, ramucirumab, galiximab, farletuzumab, ocrelizumab, ofatumumab (Arzerra®), tositumumab, ibritumomab, the CD20 antibodies 2F2 (HuMax-CD20), 7D8, IgM2C6, IgG1 2C6, 11B8, B1, 2H7, LT20, 1FS or AT80, daclizumab (Zenapax®), or anti-LHRH receptor antibodies including clone A9E4, F1G4, AT2G7, GNRH03, or GNRHR2.

115. A pharmaceutical composition comprising the conjugate of any one of claims 102 to 114 and a pharmaceutically acceptable carrier.

116. A method of treating a subject in need thereof, comprising administering to the subject a conjugate of any one of claims 102 to 114 or the pharmaceutical composition of claim 115, wherein the subject has cancer or an autoimmune disease and the conjugate binds to the target antigen associated with the cancer or autoimmune disease.

117. A Drug-Linker compound, represented by the structure of Formula (A) 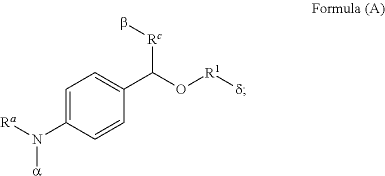
or a salt thereof, wherein:
(i) β is R²⁰(—R²¹—[O—CH₂—CH₂]ₙ₂₀—R²²—NR²⁴R²⁵)z; wherein
R²⁰ is NH, H, 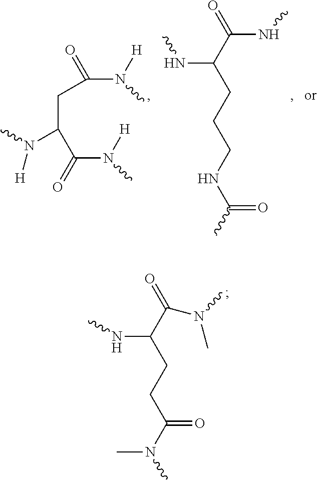
R²¹ and R²² are each, independently, a bond or C₁-C₃ alkylene;
R²⁴ and R²⁵ are each independently selected from H; polyhydroxyl group; and —C(O)— polyhydroxyl group; provided that R²⁴ and R²⁵ are not both H; z is 1, or 2; and
n20 is 2 to 26;
(ii) R^{a} is H or C₁-C₆ alkyl;
(iii) R^{c} is a bond, —C(O)—, —S(O)—, —SO₂—, C₁₋₆ alkylene, C₁₋₆ alkynylene, or C₁₋₆ alkynylene-triazolyl;
(iv) R¹ is a bond, —C(O)—, or C₁₋₆ alkylene; and
(v) δ is selected from a Drug;
(vi) α is represented by 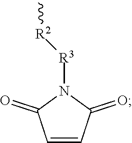
wherein
R² is a peptide having 2-5 amino acids;
R³ is —C₁-C₁₀ alkylene-C(═O)—, —C₁-C₁₀ alkylene-, —C₁-C₁₀ alkylene-C(O)NH—C₁-C₈alkylene-[O—CH₂—CH₂]ₙ—C(O)— (where n is 1 to 26), or —C₁-C₈ alkylene-(CH₂—O—CH₂)_{b}—C(═O)— (where b is 1 to 26).

118. The Drug-Linker compound of claim 117, wherein R²⁰ is

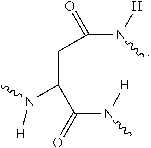

119. The Drug-Linker compound of claim 117, wherein β is 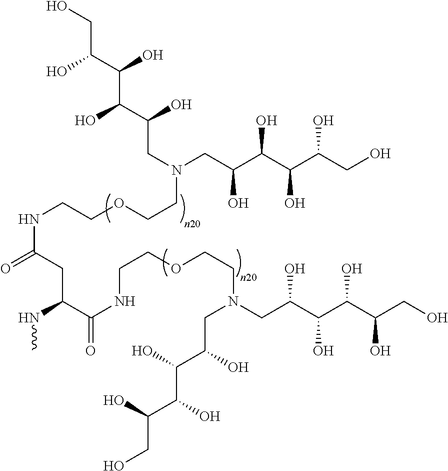
and n20 is 4 to 12.

120. The Drug-Linker compound of claim 117, wherein β is

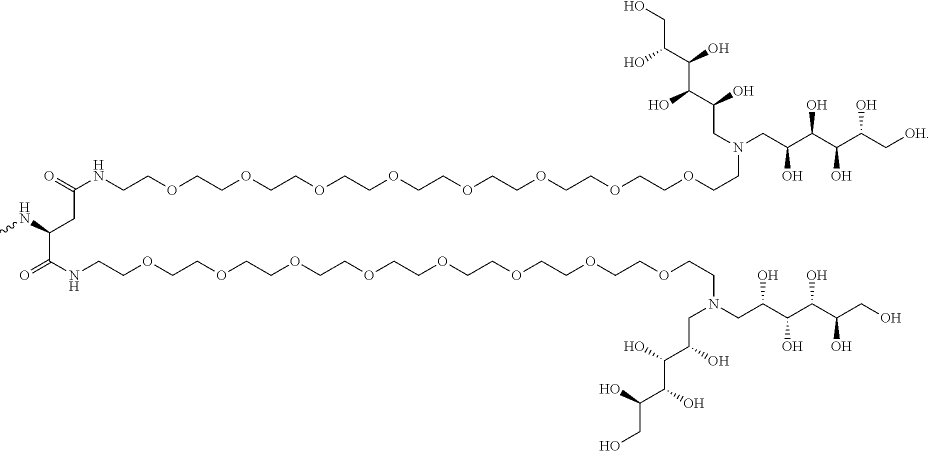

121. The Drug-Linker compound of any one of claims 117 to 120, wherein R² is selected from:

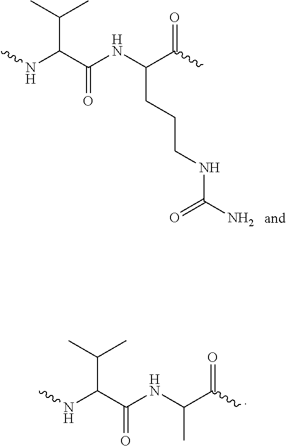

122. The Drug-Linker compound of any one of claims 117 to 121, wherein R² is selected from:

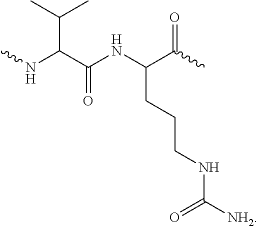

123. The Drug-Linker compound of any one of claims 117 to 122, wherein R³ is selected from:

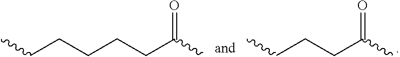

124. The Drug-Linker compound of any one of claims 117 to 123, wherein R³ is selected from:

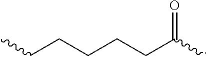

125. The Drug-Linker compound of any one of claims 117 to 124, wherein R^{c} is —C(O)—.

126. The Drug-Linker compound of any one of claims 117 to 125, wherein R¹ is —C(O)—.

127. The Drug-Linker compound of any one of claims 117 to 126, wherein the Drug is selected from a cytotoxic agent.

128. The Drug-Linker compound of any one of claims 117 to 126, wherein the Drug is MMAE or MMAF.

129. The Drug-Linker compound of any one of claims 117 to 126, wherein the Drug is MMAE.

130. The Drug-Linker compound of any one of claims 117 to 126, wherein the Drug is exatecan.

131. The Drug-Linker compound of any one of claims 117 to 126, wherein the Drug is selected from an immune modulatory agent.

132. The Drug-Linker compound of any one of claims 117 to 126, wherein δ is selected from

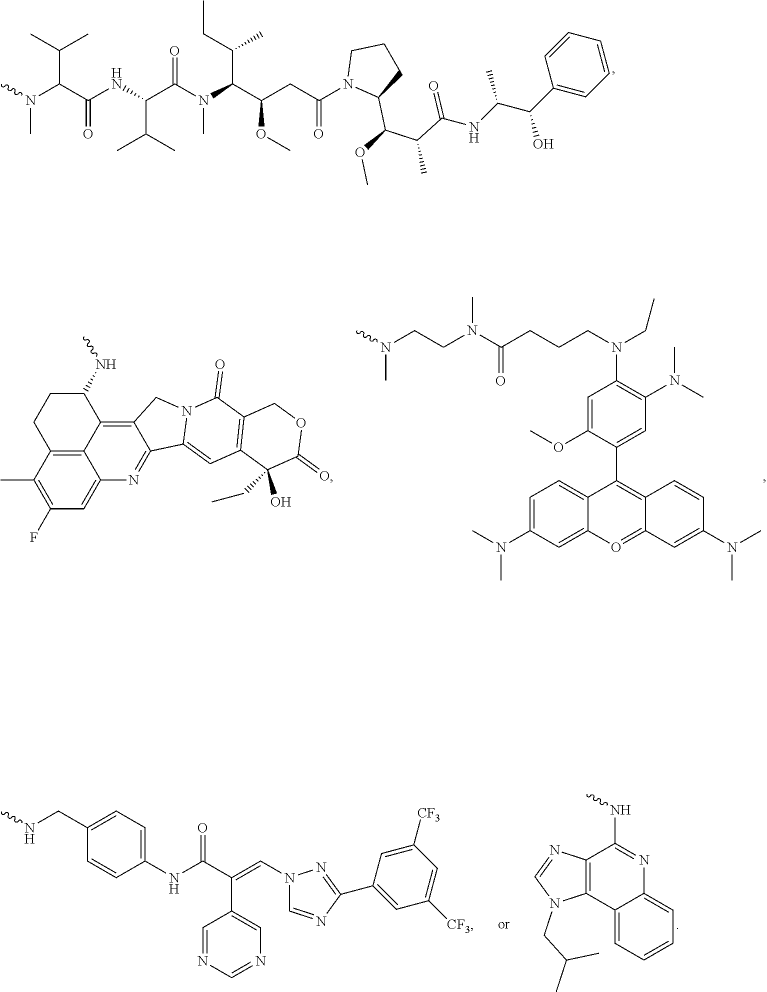

133. The Drug-Linker compound of any one of claims 117 to 126, wherein δ is

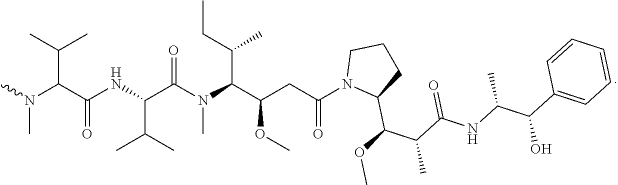

134. The Drug-Linker compound of any one of claims 117 to 126, wherein δ is from

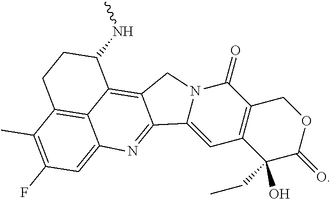

135. A conjugate comprising a Targeting group attached to the Drug-linker of any one of claims 117 to 134, wherein the Targeting group specifically binds to the target molecule.

136. The conjugate of claim 135, wherein the ratio of Drug-Linker to Targeting group is represented by a DAR value, wherein the DAR value is from 1 to 8.
